(19) United States
(12) Patent Application Publication
(10) Pub. No.: US 2024/0400555 A1
(43) Pub. Date: Dec. 5, 2024

(54) Compounds, Compositions and Methods for Attenuation of Mammalian Translation of C-MYC or N-MYC Proteins of the MYC Proto-Oncogene Family of BHLH Transcription Factors

(71) Applicant: Initial Therapeutics, Inc., South San Francisco, CA (US)

(72) Inventors: Phillip A. Patten, South San Francisco, CA (US); Jim Li, South San Francisco, CA (US); Brooke Olson Blair, South San Francisco, CA (US); Pingyu Ding, South San Francisco, CA (US); Yi Chiao Fan, South San Francisco, CA (US); Margot Paulick, South San Francisco, CA (US); Martin Linsell, South San Francisco, CA (US)

(73) Assignee: Initial Therapeutics, Inc., South San Francisco, CA (US)

(21) Appl. No.: 18/646,955

(22) Filed: Apr. 26, 2024

Related U.S. Application Data
(60) Provisional application No. 63/462,842, filed on Apr. 28, 2023.

(51) Int. Cl. C07D 471/04; A61K 31/4545; A61K 31/4709; A61K 31/4725; A61K 31/496; A61K 31/497; A61K 31/4985; A61K 31/501; A61K 31/506; A61K 31/519; A61K 31/52; A61K 31/53; A61K 31/5377; A61K 31/538; A61K 31/5383; A61K 31/541; A61K 31/55; A61K 31/553; A61K 31/695; A61K 45/06; A61P 35/00; C07D 401/12; C07D 401/14; C07D 405/14; C07D 413/14; C07D 417/14; C07D 471/10; C07D 473/00; C07D 487/04; C07D 491/052; C07D 495/04; C07D 495/20; C07D 498/04; C07D 519/00; C07F 7/18

(52) U.S. Cl. CPC C07D 471/04; A61K 31/4545; A61K 31/4709; A61K 31/4725; A61K 31/496; A61K 31/497; A61K 31/4985; A61K 31/501; A61K 31/506; A61K 31/519; A61K 31/52; A61K 31/53; A61K 31/5377; A61K 31/538; A61K 31/5383; A61K 31/541; A61K 31/55; A61K 31/553; A61K 31/695; A61K 45/06; A61P 35/00; C07D 401/12; C07D 401/14; C07D 405/14; C07D 413/14; C07D 417/14; C07D 471/10; C07D 473/00; C07D 487/04; C07D 491/052; C07D 495/04; C07D 495/20; C07D 498/04; C07D 519/00; C07F 7/1804

(57) ABSTRACT

A method of modulating c-MYC activity is disclosed. The method includes administering an effective amount of a compound of Formula I

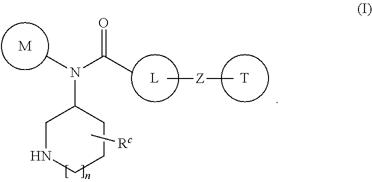

### FIELD

The present disclosure provides compounds that are inhibitors of c-MYC or n-MYC translation and are therefore useful for the treatment of cancers. Also provided are pharmaceutical compositions containing such compounds and processes for preparing such compounds.

### BACKGROUND

The expression level of c-MYC or n-MYC proteins are dysregulated in most tumor types and are key drivers of cancer progression and maintenance (e.g. in non-small-cell lung carcinoma, triple-negative breast cancer, and glioblastoma). Either c-MYC or n-MYC are known to be aberrantly expressed in up to 70% of human cancers. Both c-MYC and n-MYC are generally considered undruggable by small molecules due to their lack of a defined ligand binding sites, intrinsically disordered structure and nuclear localization. Multiple in vivo studies (Souchek 2008; Felsher 2010; Danasekaran 2020) have demonstrated that transient c-MYC inhibition results in sustained tumor regression by promoting proliferative arrest, differentiation, apoptosis, and cellular senescence in cancer cells while having minimal effects on normal tissue which are reversible. Thus targeting c-MYC presents an exciting opportunity for anti-cancer therapy.

The present disclosure is based in part on the discovery of a new approach to targeting MYC that directly circumvents these issues above. In this approach translation stallers target a cytosolic nascent chain/ribosome complex instead of a fully translated protein. Translation can be tuned to deliver partial inhibition and thus impact the therapeutic index. There are currently no effective approaches to target either c-MYC n-MYC with small molecule inhibitors and the medical opportunity is enormous with >100K deaths a year in the US attributed to c-MYC or n-MYC dysregulation.

Substituted amides have been suggested for the mediation of PCSK9 and diseases including coronary artery disease (e.g., US2014/0315928; WO2018/192493; and |https://doi.org/10.1371/journal.pbio.2001882 “Selective stalling of human translation through small-molecule engagement of the ribosome nascent chain” Nathanael Lintner et al. Mar. 21, 2017).

### SUMMARY

The following embodiments are provided.

Provided herein are methods of modulating c-MYC activity comprising administering an effective amount of a compound of Formula I, or a pharmaceutically acceptable salt thereof,

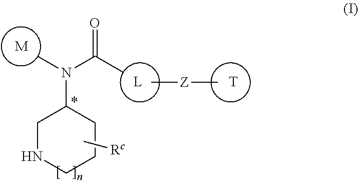

wherein
* denotes a stereocenter;
M is C₁₋₉ heteroaryl, C₁₋₉ heterocyclyl, each optionally substituted with 1, 2 or 3 substituents independently chosen from halo, substituted or unsubstituted alkyl, substituted or unsubstituted haloalkyl, substituted or unsubstituted alkenyl, substituted or unsubstituted alkynyl, substituted or unsubstituted ester, carboxylic acid, substituted or unsubstituted acetyl, substituted or unsubstituted amido, substituted or unsubstituted carbamate, substituted or unsubstituted amino, substituted or unsubstituted alkoxy, substituted or unsubstituted alkoxycarbonyl, substituted or unsubstituted thioether, cyano, substituted or unsubstituted aryl, substituted or unsubstituted heteroaryl, substituted or unsubstituted cycloalkyl, substituted or unsubstituted heterocyclyl, and combinations thereof, wherein each substituted group has 1, 2 or 3 substituents;
R^{c} is absent, C₁-C₆ alkyl, OH, NH₂, fluoro or gem-difluoro;
n is 0, 1 or 2;
L is a bond, C₆₋₁₀ arylene, C₃₋₉ heteroarylene, C₃₋₁₀ cycloalkylene, C₃₋₁₀ cycloalkenylene, or C₁₋₉ heterocyclylene, each optionally substituted with 1, 2 or 3 substituents independently chosen from halo, aryl, heteroaryl, alkyl, alkenyl, alkynyl, cycloalkyl, heterocyclyl, haloalkyl, carboxylic acid, ester, amide, alkoxy, alkoxyaryl, alkoxyheteroaryl, thioether, amino, NH-aryl, NH-heteroaryl, and cyano;
Z is a bond, —O—, S, C₁₋₃ alkylene, —(CH₂)₂C(O)NH—, —N(R^{b})—, —N(R^{b})(C₁₋₃ alkylene), where R^{b} is H, or C₁₋₃ alkyl; and
T is absent, C₆₋₁₀ aryl, C₃₋₁₀ cycloalkyl, C₂₋₉ heteroaryl, C₁₋₉ heterocyclyl, each optionally substituted with 1, 2 or 3 substituents independently chosen from OH, halo, cyano, substituted or unsubstituted aryl, substituted or unsubstituted heteroaryl, substituted or unsubstituted alkyl, haloalkyl, substituted or unsubstituted alkenyl, substituted or unsubstituted alkynyl, substituted or unsubstituted cycloalkyl, substituted or unsubstituted heterocyclyl, substituted or unsubstituted alkoxycarbonyl, hydroxycarbonyl, substituted or unsubstituted ester, substituted or unsubstituted amido, substituted or unsubstituted alkoxy, substituted or unsubstituted alkoxyaryl, substituted or unsubstituted alkoxyheteroaryl, substituted or unsubstituted thioether, substituted or unsubstituted amino, NH-aryl, and NH-heteroaryl, wherein each substituted group has 1, 2 or 3 substituents;
provided that when L is a bond, T is not absent and when T is absent, then L is not a bond.

In another embodiment, provided herein are methods of treating a condition associated with MYC activity in a subject in need thereof comprising administering to the subject an effective amount of a compound of Formula I, as defined above, or a pharmaceutically acceptable salt thereof,

In another embodiment, provided herein are methods of treating cancer in a subject in need thereof comprising administering to the subject an effective amount of a compound of Formula I, as defined above, or a pharmaceutically acceptable salt thereof.

In still other embodiments, provided herein are compounds of Formula (Ia), (Ib), (Ic), or a pharmaceutically acceptable salt thereof:

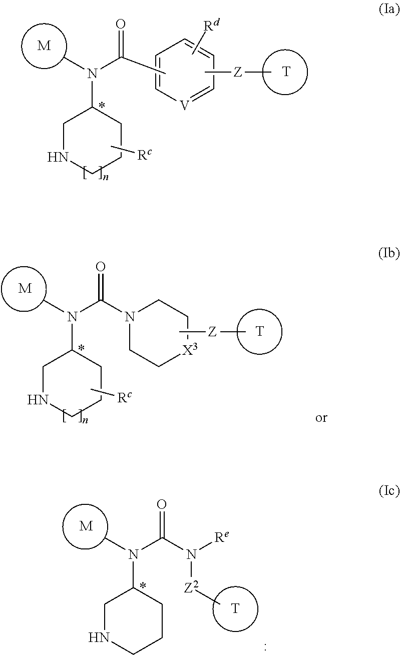

wherein
* denotes a stereocenter;
M is pyrrolo[2,3-c]pyridinyl, pyrrolo[3,2-c]pyridinyl, pyrazolo[3,4-c]pyridinyl, thieno[2,3-c]pyridinyl, thieno[3,2-c]pyridinyl, quinolinyl, isoquinolinyl, quinolinyl-2-one, 1,6-naphthyridinyl, 1,6-naphthyridinyl, pyridinyl, 2-pyrazinyl, thiazolo[5,4-c]pyridinyl, or thiazolyl, each optionally substituted with 1, 2 or 3 substituents independently chosen from halo, substituted or unsubstituted alkyl, substituted or unsubstituted haloalkyl, substituted or unsubstituted alkenyl, substituted or unsubstituted alkynyl, substituted or unsubstituted ester, carboxylic acid, substituted or unsubstituted acetyl, substituted or unsubstituted amido, substituted or unsubstituted carbamate, substituted or unsubstituted amino, substituted or unsubstituted alkoxy, substituted or unsubstituted alkoxycarbonyl, substituted or unsubstituted thioether, cyano, substituted or unsubstituted aryl, substituted or unsubstituted heteroaryl, substituted or unsubstituted cycloalkyl, substituted or unsubstituted heterocyclyl, and combinations thereof, wherein each substituted group has 1, 2 or 3 substituents;
V is N, or CH;
R^{d} is absent, halo, cyano, C₁₋₃ alkyl, or C₁₋₃ alkyloxy;
R^{c} is H, C₁-C₆ alkyl, OH, NH₂, fluoro or gem-difluoro;
n is 0, 1 or 2;
X³ is CH₂ (or CH if bonded to T through Z), NH (or N if bonded to T through Z);
R^{e} is H or C₁₋₃ alkyl;
Z² is a bond or C₁₋₃ alkylene; and
Z is a bond, C₁₋₃ alkylene, —(CH₂)₂C(O)NH—, —N(R^{b})—, where R^{b} is H, or C₁₋₃ alkyl; and
T is [1,2,3]triazolo[4,5-b]pyridinyl, pyrazolo[2,3-a]pyrimidinyl, 5,6-dihydropyrrolo[2,3-d]pyrimidinyl, pyrazolo[3,4-d]pyrimidinyl, phenyl, purinyl, pyrimidinyl, pyridinyl, pyrazinyl, pyridazinyl, pyrazolyl, tetrazolyl, 1,2,4-triazolyl, 1,2,3-triazolyl, imidazolyl, benzo[d]imidazolyl, pyrrolyl, pyrrolidinyl, isoxazolyl, isoindolyl, piperidinyl, oxetanyl, thiophenyl, morpholino, thiazolyl, pyridinyl-2-one, pyrazinyl-2-one, indazolyl, 4,5,6,7-tetrahydro-indazolyl, isoindolinyl-1,3-dione, 1,3,4-oxadiazolyl, 1,3,4-oxadiazolyl-2-one, benzo[d]isoxazolyl, benzo[d]isothiazolyl, 1,2,4-oxadiazolyl, 1,3,4-thiadiazolyl, 1,3-dihydro-2H-imidazolyl-2-one, phthalazinyl, isoquinolinyl, 2λ⁵-quinolinyl-2-one, 6,7-dihydro-4H-[1,2,3]triazolo[5,1-c][1,4]oxazinyl, 2,3-dihydro-4λ²-pyrido[3,2-b][1,4]oxazinyl, thiazolo[5,4-c]pyridinyl, or quinazolinyl, each optionally substituted with 1, 2 or 3 substituents independently chosen from OH, halo, cyano, substituted or unsubstituted aryl, substituted or unsubstituted heteroaryl, substituted or unsubstituted alkyl, haloalkyl, substituted or unsubstituted alkenyl, substituted or unsubstituted alkynyl, substituted or unsubstituted cycloalkyl, substituted or unsubstituted heterocyclyl, substituted or unsubstituted alkoxycarbonyl, hydroxycarbonyl, substituted or unsubstituted ester, substituted or unsubstituted amido, substituted or unsubstituted alkoxy, substituted or unsubstituted alkoxyaryl, substituted or unsubstituted alkoxyheteroaryl, substituted or unsubstituted thioether, substituted or unsubstituted amino, NH-aryl, and NH-heteroaryl, wherein each substituted group has 1, 2 or 3 substituents;
provided that when the compound is of Formula (Ia) then
i) when n is 1, R^{c} is absent, R^{d} is absent, Z is a bond, and M is 3-chloropyridin-2-yl or 3-methylpyridin-2-yl, then T is not unsubstituted pyrazolo[2,3-a]pyrimidinyl; methyl substituted pyrazolo[2,3-a]pyrimidinyl; unsubstituted [1,2,3]triazolo[4,5-b]pyridinyl; methyl substituted [1,2,3]triazolo[4,5-b]pyridinyl; or pyrazolyl substituted with a methyl group, a substituted carboxyl group, a substituted or unsubstituted tetrazolyl group, or a substituted or unsubstituted 1,3,4-oxadiazolyl-2-one group; or
ii) when n is 1, R^{c} is absent, R^{d} is absent, Z is a bond, and M is unsubstituted isoquinolinyl or methyl substituted pyrrolo[2,3-c]pyridinyl, then T is not a pyrazolyl substituted with methyl, substituted carboxyl or both methyl and substituted carboxyl; or
iii) when M is phenyl or pyridinyl, and Z is a bond, then T is not 5,5-dimethylisoxazolyl-4(5H)-one or 4-alkoxy-5,5-dimethyl-4,5-dihydroisoxazolyl.

### DETAILED DESCRIPTION

The following description sets forth exemplary embodiments of the present technology. It should be recognized, however, that such description is not intended as a limitation on the scope of the present disclosure but is instead provided as a description of exemplary embodiments.

### I. DEFINITIONS

### Abbreviations

BHLH. Basic helix-loop-helix.

EC50. “Effective concentration”, as defined below.

pEC50. Negative logarithm base 10 of the EC50.

IC50. “Inhibitory concentration 50” as defined below.

pIC50. Negative logarithm base 10 of the IC50.

DNA. Deoxyribonucleic acid.

RNA. Ribonucleic acid.

mRNA. Messenger RNA. This is typically comprised of the following elements in order starting from the 5′ end of the mRNA: mRNA CAP, 5′-untranslated region, initiation codon, “open reading frame”, stop codon, and 3′ untranslated region.

5′-mRNA CAP: A specially altered nucleotide on the 5′ end of an mRNA molecule, which consists of a guanine nucleotide connected to mRNA via a 5′ to 5′ triphosphate linkage.

LC. Liquid chromatography.

LLOQ. Lower limit of quantification, as described below.

MAX. The protein encoded by this gene is a member of the basic helix-loop-helix leucine zipper (bHLHZ) family of transcription factors. It is able to form homodimers and heterodimers with other family members, which include Mad, Mxil and Myc. The underlying gene structure is further described here: https://www.ncbi.nlm.nih.gov/gene/4149.

MS. Mass spectrometry.

MYC. The family of proto-oncogenes comprised of c-MYC, n-MYC and 1-MYC, as defined and described below.

C-MYC. The c-MYC gene is a protooncogene whose dysregulation due to rearrangement, copy number amplification and other forms of dysregulation (i.e. mutations in the Wnt pathway the result in upregulation of c-MYC) are correlated with many forms of cancer. The underlying gene structure is further described here: https://www.ncbi.nlm.nih.gov/gene/4609.

N-MYC. The n-MYC gene is a protooncogene whose dysregulation due to rearrangement, copy number amplification and other forms of dysregulation are correlated with many forms of cancer, notably neuroblastoma. The underlying gene structure is further described here: https://www.ncbi.nlm.nih.gov/gene/4613

L-MYC. The 1-MYC gene is a protooncogene whose dysregulation due to rearrangement, copy number amplification and other forms of dysregulation are correlated with many forms of cancer, notably neuroblastoma. The underlying gene structure is further described here: https://www.ncbi.nlm.nih.gov/gene/4610.

MM. Multiple myeloma.

TMT. Tandem Mass Tag.

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line or a dashed line drawn through a line in a Formula indicates a specified point of attachment of a group. Unless chemically or structurally required, no directionality or stereochemistry is indicated or implied by the order in which a chemical group is written or named.

The prefix “Cᵤ₋ᵥ” indicates that the following group has from u to v carbon atoms. For example, “C₁₋₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount±10%. In certain embodiments, the term “about” includes the indicated amount±5%. In certain embodiments, the term “about” includes the indicated amount±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁₋₂₀ alkyl), 1 to 12 carbon atoms (i.e., C₁₋₁₂ alkyl), 1 to 8 carbon atoms (i.e., C₁₋₈ alkyl), 1 to 6 carbon atoms (i.e., C₁₋₆ alkyl) or 1 to 4 carbon atoms (i.e., C₁₋₄ alkyl). Examples of alkyl groups include methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂) and tert-butyl (i.e., —C(CH₃)₃); and “propyl” includes n-propyl (i.e., —(CH₂)₂CH₃) and isopropyl (i.e., —CH(CH₃)₂).

Certain commonly used alternative chemical names may be used. For example, a divalent group such as a divalent “alkyl” group, a divalent “aryl” group, etc., may also be referred to as an “alkylene” group, “alkylenyl” group, or an “alkenyl” group, an “arylene” group or an “arylenyl” group, respectively. In another example, a trivalent group such as a trivalent “alkyl” group may also be referred to as an “alkylyne” group, “alkylynyl” group or “alkynyl” group. Also, unless indicated explicitly otherwise, where combinations of groups are referred to herein as one moiety, e.g. arylalkyl or aralkyl, the last mentioned group contains the atom by which the moiety is attached to the rest of the molecule.

“Alkoxy” refers to the group “alkyl-O—”. Examples of alkoxy groups include methoxy, ethoxy, n-propoxy, iso-propoxy, n-butoxy, tert-butoxy, sec-butoxy, n-pentoxy, n-hexoxy and 1,2-dimethylbutoxy.

“Alkoxycarbonyl” refers to the group “alkyl-O—C(O)”.

“Amide” or “amido” refers to the group “R—C(O) NRR”.

“Amino” refers to the group —NR^{y}R^{z} wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Aryl” refers to an aromatic carbocyclic group having a single ring (e.g. monocyclic) or multiple rings (e.g. bicyclic or tricyclic) including fused systems. As used herein, aryl has 6 to 20 ring carbon atoms (i.e., C₆₋₂₀ aryl), 6 to 18 carbon ring atoms (i.e., C₆₋₁₈ aryl), 6 to 12 carbon ring atoms (i.e., C₆₋₁₂ aryl) or 6 to 10 carbon ring atoms (i.e., C₆₋₁₀ aryl). Examples of aryl groups include phenyl, naphthyl, fluorenyl and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl.

“Cycloalkyl” refers to a saturated or partially unsaturated cyclic alkyl group having a single ring or multiple rings including fused, bridged and spiro ring systems. The term “cycloalkyl” includes cycloalkenyl groups (i.e., the cyclic group having at least one double bond). As used herein, cycloalkyl has from 3 to 20 ring carbon atoms (i.e., C₃₋₂₀) cycloalkyl), 3 to 15 ring carbon atoms (i.e., C₃₋₁₅ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl) or 3 to 6 ring carbon atoms (i.e., C₃₋₆ cycloalkyl). Cycloalkyl also includes “spiro cycloalkyl” when there are two positions for substitution on the same carbon atom. Monocyclic radicals include, for example, cyclopropyl, cyclobutyl, cyclopentyl, cyclohexyl, cycloheptyl and cyclooctyl. Polycyclic radicals include, for example, adamantyl, norbornyl, decalinyl, 7,7-dimethyl-bicyclo[2.2.1]heptanyl and the like. Further, the term cycloalkyl is intended to encompass any non-aromatic ring which may be fused to an aryl ring, regardless of the attachment to the remainder of the molecule.

“Carboxylic acid” and “hydroxycarbonyl” each refer to the group “R—COOH”.

“Cyano” or “Carbonitrile” refers to a —CN group.

“Ester” refers to the group “R—C(O)O—R”.

“Halogen” or “halo” includes fluoro, chloro, bromo, and iodo.

“Haloalkyl” refers to an alkyl group as defined herein wherein one ore more of the hydrogen atoms of the alkyl group are replace by a halogen group.

“Heteroalkyl” refers to an alkyl group in which one or more of the carbon atoms (and any associated hydrogen atoms) are each independently replaced with the same or different heteroatomic group, provided the point of attachment to the remainder of the molecule is through a carbon atom. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NR—, —O—, —S—, —S(O)—, —S(O)₂—, and the like, where R is hydrogen, alkyl, alkenyl, alkynyl, cycloalkyl, heterocyclyl, aryl, heteroalkyl, or heteroaryl; each of which may be optionally substituted, as defined herein. Examples of heteroalkyl groups include —CH₂OCH₃, —CH₂SCH₃, and —CH₂NRCH₃, where R is hydrogen, alkyl, alkenyl, alkynyl, cycloalkyl, heterocyclyl, aryl, heteroalkyl, or heteroaryl; each of which may be optionally substituted, as defined herein. As used herein, heteroalkyl includes 1 to 10 carbon atoms, 1 to 8 carbon atoms, or 1 to 4 carbon atoms; and 1 to 3 heteroatoms, 1 to 2 heteroatoms, or 1 heteroatom.

“Heteroaryl” refers to an aromatic group (e.g., a 5-14 membered ring system) having a single ring, multiple rings or multiple fused rings, with one or more ring heteroatoms independently selected from nitrogen, oxygen and sulfur. As used herein, heteroaryl includes 1 to 20 ring carbon atoms (i.e., C₁₋₂₀ heteroaryl), 1 to 13 ring carbon atoms (i.e., C₃₋₁₂ heteroaryl), 3 to 12 ring carbon atoms (i.e., C₃₋₁₂ heteroaryl) or 3 to 8 carbon ring atoms (i.e., C₃₋₈ heteroaryl); and 1 to 6 heteroatoms, 1 to 5 heteroatoms, 1 to 4 heteroatoms, 1 to 3 ring heteroatoms, 1 to 2 ring heteroatoms or 1 ring heteroatom independently selected from nitrogen, oxygen and sulfur. Examples of heteroaryl groups include azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl and thiophenyl (i.e., thienyl). Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocyclyl” refers to a saturated or unsaturated cyclic alkyl group, with one or more ring heteroatoms independently selected from nitrogen, oxygen and sulfur. The term “heterocyclyl” includes heterocycloalkenyl groups (i.e., the heterocyclyl group having at least one double bond), bridged-heterocyclyl groups, fused-heterocyclyl groups and spiro-heterocyclyl groups. A heterocyclyl may be a single ring or multiple rings wherein the multiple rings may be fused, bridged or spiro, and may comprise one or more oxo (C═O) or N-oxide (N—O—) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 2 to 20 ring carbon atoms (i.e., C₂₋₂₀ heterocyclyl), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋₈ heterocyclyl), 3 to 12 ring carbon atoms (i.e., C₃₋₁₂ heterocyclyl), 3 to 8 ring carbon atoms (i.e., C₃₋₈ heterocyclyl) or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms or 1 ring heteroatom independently selected from nitrogen, sulfur or oxygen. Examples of heterocyclyl groups include dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl and 1,1-dioxo-thiomorpholinyl. Examples of the spiro-heterocyclyl rings include bicyclic and tricyclic ring systems, such as 2-oxa-7-azaspiro[3.5]nonanyl, 2-oxa-6-azaspiro[3.4]octanyl and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl and isoindolinyl, where the heterocyclyl can be bound via either ring of the fused system.

“Thioether” refers to the group “R—S—R”.

“Cyclic organic radical” refers to a cyclic organic moiety which may be for example an aryl, heteroaryl, cycloalkyl or heterocyclyl moiety or one or more of said cyclic moieties bonded, spiro-linked or fused together, any of which may be optionally substituted with one or more substituents which optional substituents may also be optionally substituted.

The terms “optional” or “optionally” means that the subsequently described event or circumstance may or may not occur and that the description includes instances where said event or circumstance occurs and instances in which it does not. Also, the term “optionally substituted” refers to any one or more hydrogen atoms on the designated atom or group may or may not be replaced by a moiety other than hydrogen. As used herein unless otherwise indicated an optionally substituted group may have 0, 1, 2, or 3 hydrogen atoms replaced by another group.

Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl) substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein. Unless specified otherwise, where a group is described as optionally substituted, any substituents of the group are themselves unsubstituted. For example, in certain embodiments, the term “substituted alkyl” refers to an alkyl group having one or more substituents including hydroxy, halo, alkoxy, acyl, oxo, amino, cycloalkyl, heterocyclyl, aryl and heteroaryl. In certain embodiments, the one or more substituents may be further substituted with halo, alkyl, haloalkyl, hydroxy, alkoxy, cycloalkyl, heterocyclyl, aryl or heteroaryl, each of which is substituted. In certain embodiments, the substituents may be further substituted with halo, alkyl, haloalkyl, alkoxy, hydroxy, cycloalkyl, heterocyclyl, aryl or heteroaryl, each of which is unsubstituted.

The compounds described herein may in some cases exist as diastereomers, enantiomers, or other stereoisomeric forms. All chiral, diastereomeric, racemic forms, as individual forms and mixtures thereof, are within the scope of this disclosure, unless the specific stereochemistry or isomeric form is specifically indicated. Compounds of the present disclosure containing an asymmetrically substituted atom may be isolated in optically active, optically enriched, optically pure, or racemic forms. It is well known in the art how to prepare optically active forms, such as by resolution of materials. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley and Sons, Inc., 1981, herein incorporated by reference for this disclosure). Stereoisomers may also be obtained by stereoselective synthesis.

Certain compounds of Formula (I) (or any of the embodiments thereof described herein) and/or a pharmaceutically acceptable salt, solvate, hydrate, or prodrug thereof can exist as tautomers and/or geometric isomers. All possible tautomers and cis and trans isomers, as individual forms and mixtures thereof, are within the scope of this disclosure. For example, pyrazole tautomers as shown below are equivalent structures. The depiction of one such structure is intended to encompass both structures.

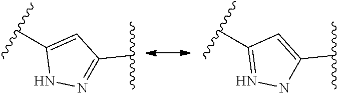 [SMILES: *c1cc(*)[nH]n1 ; *c1cc(*)[nH]n1]

In some embodiments the compounds of Formula (I) may present in two or more rotamer forms, for example around the central tertiary amide bond. In some embodiments of compounds of Formula (I) with a sterically hindered substitution on the amide in Formula (I), for example compounds of Formula (I) with the 8-position substitution on an isoquinoline M and/or with substitution on the L group, show more than one set of signals in either ¹H NMR and/or HPLC.

Any compound or Formula given herein, such as the compounds of Formula I, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I and ¹²⁵I, respectively. Various isotopically labeled compounds of the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes “deuterated analogs” of compounds described herein in which from 1 to n hydrogens attached to a carbon atom is/are replaced by deuterium, in which n is the number of hydrogens in the molecule. Such compounds exhibit increased resistance to metabolism and are thus useful for increasing the half-life of any compound when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F, ³H, ¹¹C labeled compound may be useful for PET or SPECT or other imaging studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base salts by virtue of the presence of amino and/or carboxyl groups or groups similar thereto.

Provided are also pharmaceutically acceptable salts, hydrates, solvates, tautomeric forms, stereoisomers and prodrugs of the compounds described herein, such as the compounds of Formula I, and are included within the term “compounds”. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃) or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine and the like.

Certain compounds of the present invention or combination agents may exist in more than one crystal form (generally referred to as “polymorphs”). Polymorphs may be prepared by crystallization under various conditions, for example, using different solvents or different solvent mixtures for recrystallization; crystallization at different temperatures; and/or various modes of cooling, ranging from very fast to very slow cooling during crystallization. Polymorphs may also be obtained by heating or melting the compound of the present invention followed by gradual or fast colling. The presence of polymorphs may be determined by solid probe NMR spectroscopy, IR spectroscopy, differential scanning calorimetry, powder X-ray diffraction or such other techniques.

The term “hydrate” refers to the complex formed by the combining of a compound described herein and water.

A “solvate” refers to an association or complex of one or more solvent molecules and a compound of the disclosure. Examples of solvents that form solvates include, but are not limited to, water, isopropanol, ethanol, methanol, dimethylsulfoxide, ethylacetate, acetic acid and ethanolamine.

The compounds disclosed herein, such as the compounds of Formula I, or their pharmaceutically acceptable salts include an asymmetric center and may thus give rise to enantiomers, diastereomers, and other stereoisomeric forms that may be defined, in terms of absolute stereochemistry, as (R)- or(S)— or, as (D)- or (L)- for amino acids. The disclosure is meant to include all such possible isomers, as well as their racemic and optically pure forms. Optically active (+) and (−), (R)- and(S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposable mirror images of one another.

“Diastereoisomers” or “diastereomers” are stereoisomers that have at least two asymmetric atoms, but which are not mirror-images of each other.

“Rotamers” are conformational isomers that differ by rotation about only a single sigma bond.

“Prodrugs” means any compound which releases an active parent drug according to a Formula described herein in vivo when such prodrug is administered to a mammalian subject. Prodrugs of a compound described herein are prepared by modifying functional groups present in the compound described herein in such a way that the modifications may be cleaved in vivo to release the parent compound. Prodrugs may be prepared by modifying functional groups present in the compounds in such a way that the modifications are cleaved, either in routine manipulation or in vivo, to the parent compounds. Prodrugs include compounds described herein wherein a hydroxy, amino, carboxyl or sulfhydryl group in a compound described herein is bonded to any group that may be cleaved in vivo to regenerate the free hydroxy, amino or sulfhydryl group, respectively. Examples of prodrugs include, but are not limited to esters (e.g., acetate, formate and benzoate derivatives), amides, guanidines, carbamates (e.g., N,N-dimethylaminocarbonyl) of hydroxy functional groups in compounds described herein and the like. Preparation, selection and use of prodrugs is discussed in T. Higuchi and V. Stella, “Pro-drugs as Novel Delivery Systems,” Vol. 14 of the A.C.S. Symposium Series; “Design of Prodrugs,” ed. H. Bundgaard, Elsevier, 1985; and in Bioreversible Carriers in Drug Design, ed. Edward B. Roche, American Pharmaceutical Association and Pergamon Press, 1987, each of which are hereby incorporated by reference in their entirety.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response or other problem or complication, commensurate with a reasonable benefit/risk ratio.

As used herein, “pharmaceutically acceptable carrier” or “pharmaceutically acceptable excipient” or “excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

As used herein, the terms “administering” and “administered” refer to the delivery of a composition into a subject by a method or route that results in at least partial localization of the composition at a desired site. A composition can be administered by any appropriate route that results in effective treatment in the subject, i.e., administration results in delivery to a desired location in the subject where at least a portion of the composition is delivered to the desired site for a period of time. Modes of administration include injection, infusion, instillation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebrospinal, and intrasternal injection and infusion. In some examples, the route is intravenous.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition and/or diminishing the extent of the disease or condition); b) slowing or arresting the development of one or more clinical symptoms associated with the disease or condition (e.g., stabilizing the disease or condition, preventing or delaying the worsening or progression of the disease or condition and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life and/or prolonging survival).

“Prevention” or “preventing” means any treatment of a disease or condition that causes the clinical symptoms of the disease or condition not to develop. Compounds may, in certain embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In certain embodiments, the subject is a mammal. In certain embodiments, the subject is a human.

The term “therapeutically effective amount” or “effective amount” of a compound described herein or a pharmaceutically acceptable salt, prodrug, stereoisomer or a mixture of stereoisomers thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition of as described herein. The therapeutically effective amount may vary depending on the subject and disease or condition being treated, the weight and age of the subject, the severity of the disease or condition and the manner of administering, which can readily be determined by one of ordinary skill in the art.

As defined herein “Inhibition of Translation” refers to inhibition of the rate of translational elongation of an mRNA to a corresponding polypeptide by the ribosome. This mode of inhibition may include a compound inducing the action of processes collectively known as Ribosomal Quality Control in a compound- and mRNA-dependent manner. This mode of inhibition contrasts with inhibition of initiation of translation at a “start codon” and termination of translation at a “stop codon”. This mode of inhibition is also in contrast to inhibition of gene expression at an “upstream” step such as transcription of DNA to RNA or splicing of pre-mRNA to mRNA. Direct inhibition may be determined by the “In Vitro Single Point Translation Screening Assay” provided herein (Example 1). This “In Vitro Translation Single Point Screening Assay” is specific to the production of protein from mRNA and therefore detects inhibitors of this translational process rather than other mechanisms by which protein accumulation may be reduced. Any compound that presents an IC₅₀ (μM) below about 50 μM in the “In Vitro Translation Single Point Screening Assay” is considered as inhibiting translation. In some embodiments, the IC₅₀ of the compound is less than about 30 μM, or the IC₅₀ of the compound is less than about 20 μM.

As used herein “modulating, modulate or modulated” refers to modulation of protein accumulation in either an in vitro or in a cellular context. Modulation may be measured in an in vitro biochemical assay using the In Vitro Single Point Translation Screening Assay provided herein or by direct proteomic assays such as Western blots, alpha screen or mass spec analysis. Modulation may also be measured in a cellular context using Western blots, alpha screen, or other proteomic assay methods such as mass spec analysis. Any compound that modulates the accumulation of c-MYC or n-MYC protein in a cellular context will indirectly modulate the expression level of downstream genes that are controlled by the respective c-MYC or n-MYC transcription factors. The c-MYC or n-MYC oncoproteins potentially regulate the transcription of at least 15% of the entire genome (Chen 2018).

A biomarker is a measurable indicator of the severity or presence of some disease state as well as an indicator of the mechanism of action of a drug of interest. More generally a biomarker is anything that can be used as an indicator of a particular disease state or some other physiological state of an organism. According to the WHO, the indicator may be chemical, physical, or biological in nature- and the measurement may be functional, physiological, biochemical, cellular, or molecular. A biomarker can be a substance whose detection indicates a particular disease state or a substance for which a change in expression or state of a protein correlates with the risk or progression of a disease, or with the susceptibility of the disease to a given treatment. Biomarkers can be characteristic biological properties or molecules that can be detected and measured in parts of the body like the blood or tissue. They may indicate either normal or diseased processes in the body. Biomarkers can be specific cells, molecules, gene rearrangements (as with Burkitt's lymphoma), gene products, enzymes, or hormones. Biomarkers are useful in a number of ways, including mechanism of action of the drug of interest, measuring the progress of disease, evaluating the most effective therapeutic regimes for a particular cancer type, and establishing long-term susceptibility to cancer or its recurrence. Biomarkers play roles in medicinal biology and help in early diagnosis, disease prevention, drug target identification, drug response etc. As used herein the phrase “biomarker that predicts anti-MYC activity” refers to a biomarker as described above associated with modulation of translation of c-MYC or n-MYC.

As used herein, “effective concentration₅₀ (EC₅₀)” refers to the concentration at which a given compound exerts 50% of the maximum effect observed for that compound. This concentration is typically estimated using Prism Graphpad or an equivalent software tool that infers the EC50 from a multi-point dose response curve. In a typical EC50 estimation there is region at the low concentration end of the dose response curve showing no significant change relative to the DMSO control. This is the so-called “top” of the dose response curve. At high concentrations, there is a “top” where the effect is observed to plateau to a maximum value. The maximum value for inhibition is often defined using a known inhibitor of the pathway dosed at a concentration that is 10× or move above the EC50 for the known inhibitor. The EC50 refers to the concentration that is mathematically inferred by a software program such as Prism Graphpad to give a 50% of maximum effect. As used herein the phrase “activates c-MYC or n-MYC activity” refers to direct activation versus, for example, upstream activation. Direct activation is determined in some instances by the In Vitro Multiple Point Translation Screening Assay. Any compound that results in at least 2× increased expression of MYC relative to the DMSO control and presents an Effective Concentration (EC₅₀) below about 50 μM in the In Vitro Multiple Point Translation Screening Assay is considered as activating translation. In some embodiments, the EC₅₀ of the compound is less than about 30 μM, or the EC₅₀ of the compound is less than about 20 μM.

As used herein the phrase “condition associated with c-MYC or n-MYC activity” refers to at least those conditions disclosed herein, especially in the methods section, and any other conditions known to be mediated, caused, moderated or associated with c-MYC or n-MYC activity, for example the GeneCards Summary for c-MYC Gene indicates that diseases associated with MYC include Burkitt Lymphoma and High-Grade B-Cell Lymphoma Double-Hit/Triple-Hit.

In some embodiments, the compound “selectively” inhibits translation of mRNA to protein. The terms “selective” or “selectively” are defined as “significant inhibition” of translation of less than 10 percentage of proteins in a typical “Global Proteomic Assay”. As used herein, “Global Proteomic Assay” refers to an assay wherein the expression level of all proteins is assessed without utilizing a protein-specific detection method such as is used in Western blots, alpha screen assays or ELISA assays. In contrast to protein-specific proteomic methods, a Global proteomic Assay is designed to quantitatively measure the levels of all proteins that are observed above the Lower Limit of Quantification (LLOQ) of the detection method used in the protocol. The LLOQ is the lowest value of a signal (or concentration, activity, response, etc. . . . ) that can be quantified with acceptable precision and accuracy in an assay of interest.

A typical Global Proteomic Assay utilizes liquid chromatography of trypsinized whole cellular extracts, together with mass spectrometry to assess the relative abundance of all tryptic peptides that are present within the Limit of Quantification for the particular instruments used in the assay. A typical Global Proteomic Assay uses Tandem Mass Tags (TMT) and is described in Rauniyar N. et al. 2014 and Deracinois B. et al. 2013. It is preferred that the level is below about 1% of proteins and especially preferred that the level is below about 0.5% or 0.1% of proteins. Typically, in a standard assay the 1% level equates to about 40 non-target proteins out of about 4000 proteins for which statistically significant measurements can be made. “Significant inhibition” of a particular protein in a Global Proteomic Assay is defined as a decrease in protein expression in the Global Proteomic Assay to below about 50% of its starting level relative to vehicle. This definition of “inhibition related to the Global Proteomic Assay” is not to be confused with the previous definition of inhibition related to the In Vitro Single Point Translation Screening Assay or the In Vitro Multiple Point Translation Screening Assay.

As used herein, Ribosome Profiling Assay is an assay in which one measures the relative abundance of sequences of mRNA present in cellular extracts prepared under specified conditions that are protected from nuclease digestion due to occupancy by ribosomes. A typical Ribosome Profiling Assay is described here: https://ribomaps.com/services. In a typical application, cells are treated with either vehicle or vehicle plus compound of interest at a concentration known to have measurable cellular effects and extracts are prepared at a specified time point where measurable activity is expected. The extracts are subjected to Ribosome Profiling, an analysis wherein the sequencing results are used to infer the locations on mRNA of ribosomes in the cellular extracts at the specified timepoints with the specified treatments. The data can be used to infer the locations on mRNA of compound-dependent translationally stalling.

### II. COMPOUNDS

Provided herein are compounds of Formula I, or a pharmaceutically acceptable salt thereof,

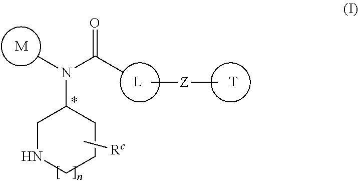

wherein
* denotes a stereocenter;
M is C₁₋₉ heteroaryl, C₁₋₉ heterocyclyl, each optionally substituted with 1, 2 or 3 substituents independently chosen from halo, substituted or unsubstituted alkyl, substituted or unsubstituted haloalkyl, substituted or unsubstituted alkenyl, substituted or unsubstituted alkynyl, substituted or unsubstituted ester, carboxylic acid, substituted or unsubstituted acetyl, substituted or unsubstituted amido, substituted or unsubstituted carbamate, substituted or unsubstituted amino, substituted or unsubstituted alkoxy, substituted or unsubstituted alkoxycarbonyl, substituted or unsubstituted thioether, cyano, substituted or unsubstituted aryl, substituted or unsubstituted heteroaryl, substituted or unsubstituted cycloalkyl, substituted or unsubstituted heterocyclyl, and combinations thereof, wherein each substituted group has 1, 2 or 3 substituents;
R^{c} is absent, C₁-C₆ alkyl, OH, NH₂, fluoro or gem-difluoro;
n is 0, 1 or 2;
L is a bond, C₆₋₁₀ arylene, C₃₋₉ heteroarylene, C₃₋₁₀ cycloalkylene, C₃₋₁₀ cycloalkenylene, or C₁₋₉ heterocyclylene, each optionally substituted with 1, 2 or 3 substituents independently chosen from halo, aryl, heteroaryl, alkyl, alkenyl, alkynyl, cycloalkyl, heterocyclyl, haloalkyl, carboxylic acid, ester, amide, alkoxy, alkoxyaryl, alkoxyheteroaryl, thioether, amino, NH-aryl, NH-heteroaryl, and cyano;
Z is a bond, —O—, S, C₁₋₃ alkylene, —(CH₂)₂C(O)NH—, —N(R^{b})—, —N(R^{b})(C₁₋₃ alkylene), where R^{b} is H, or C₁₋₃ alkyl; and
T is absent, C₆₋₁₀ aryl, C₃₋₁₀ cycloalkyl, C₂₋₉ heteroaryl, C₁₋₉ heterocyclyl, each optionally substituted with 1, 2 or 3 substituents independently chosen from OH, halo, cyano, substituted or unsubstituted aryl, substituted or unsubstituted heteroaryl, substituted or unsubstituted alkyl, haloalkyl, substituted or unsubstituted alkenyl, substituted or unsubstituted alkynyl, substituted or unsubstituted cycloalkyl, substituted or unsubstituted heterocyclyl, substituted or unsubstituted alkoxycarbonyl, hydroxycarbonyl, substituted or unsubstituted ester, substituted or unsubstituted amido, substituted or unsubstituted alkoxy, substituted or unsubstituted alkoxyaryl, substituted or unsubstituted alkoxyheteroaryl, substituted or unsubstituted thioether, substituted or unsubstituted amino, NH-aryl, and NH-heteroaryl, wherein each substituted group has 1, 2 or 3 substituents;
provided that when L is a bond, T is not absent and when T is absent, then L is not a bond.

In some embodiments, pyrrolo[2,3-c]pyridinyl, pyrrolo[3,2-c]pyridinyl, pyrazolo[3,4-c]pyridinyl, thieno[2,3-c]pyridinyl, thieno[3,2-c]pyridinyl, quinolinyl, isoquinolinyl, quinolinyl-2-one, 1,6-naphthyridinyl, 1,6-naphthyridinyl, pyridinyl, or 2-pyrazinyl, each optionally substituted with 1, 2 or 3 substituents independently chosen from halo, substituted or unsubstituted alkyl, substituted or unsubstituted haloalkyl, substituted or unsubstituted alkenyl, substituted or unsubstituted alkynyl, ester, carboxylic acid, substituted or unsubstituted amido, substituted or unsubstituted amino, substituted or unsubstituted alkoxy, substituted or unsubstituted thioether, cyano, substituted or unsubstituted aryl, substituted or unsubstituted heteroaryl, substituted or unsubstituted cycloalkyl, substituted or unsubstituted heterocyclyl, and combinations thereof, wherein each substituted group has 1, 2 or 3 substituents.

In some embodiments, M is

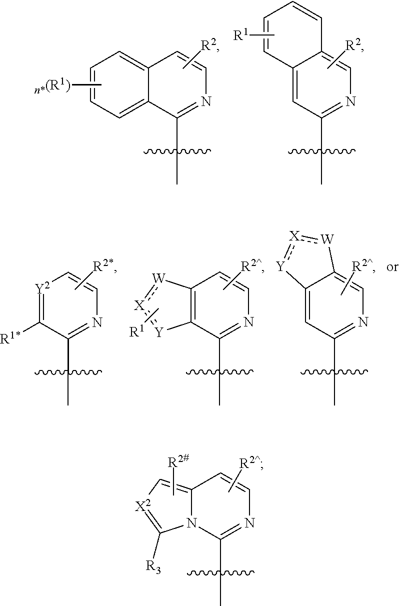

wherein
each R¹ is independently absent halo, substituted or unsubstituted alkyl, substituted or unsubstituted haloalkyl, substituted or unsubstituted alkenyl, substituted or unsubstituted alkynyl, substituted or unsubstituted aryl, substituted or unsubstituted ester, substituted or unsubstituted acetyl, carboxylic acid, substituted or unsubstituted alkoxycarbonyl, substituted or unsubstituted amido, substituted or unsubstituted carbamate; substituted or unsubstituted alkoxy, substituted or unsubstituted thioether, substituted or unsubstituted amino, cyano, substituted or unsubstituted heteroaryl, substituted or unsubstituted cycloalkyl or substituted or unsubstituted heterocyclyl, wherein each substituted group has 1, 2 or 3 substituents;
n* is 0, 1, 2 or 3;

R² is absent, halo, alkyl, alkenyl, alkynyl, aryl heteroaryl or cyano;

R^{1*} is absent, halo, alkyl, alkenyl, alkynyl, aryl, ester, carboxylic acid, amide, alkoxy, thioether, cyano, heteroaryl, cycloalkyl or heterocyclyl;
R^{2*} is absent, halo, alkyl, alkenyl, alkynyl, aryl, ester, carboxylic acid, amide, alkoxy, thioether, cyano, heteroaryl, cycloalkyl or heterocyclyl; and each W, X, and Y are independently —CH, —N, —NH, N-alkyl, O, or S;
R^{2#} is absent, halo, alkyl, alkenyl, alkynyl, aryl, ester, carboxylic acid, amide, alkoxy, thioether, amino, cyano, heteroaryl, cycloalkyl or heterocyclyl;
X² is CH, N, C-alkyl, C-alkenyl, C-alkynyl, C-aryl or C-heteroaryl;
Y² is CH or N;
R^{2\hat{ }} is absent, alkyl, alkenyl, alkynyl, cyano or halo; and
R₃ is absent, alkyl, alkenyl, alkynyl, halo, cyano, ester, carboxylic acid, amide, aryl, heteroaryl, alkoxy, thioether, or amino; and
[symbol] is a bond that is a double bond or a single bond.

In some embodiments, M is

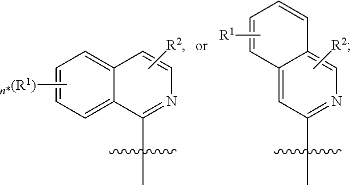

wherein
each R¹ is independently absent, cyano; chloro; bromo; fluoro; acetyl; amido; carbamate; alkylcarbamate; pyrazinyl; tetrazolyl; hydroxycarbonyl; alkoxycarbonyl; pyridinyl-2-one; C₁₋₆ alkoxy; C₁₋₆ haloalkyl; C₁₋₆ cycloalkyl; C₁₋₆ alkyl optionally substituted with OH, pyridinyl, amido, or phenyl; C₂₋₆ alkenyl optionally substituted with amido, OH, pyrazolyl, pyridinyl, isoxazolyl, thiophenyl, alkyl substituted amido, hydroxyalkylamido, arylalkylamido, alkoxycarbonyl, or hydroxycarbonyl; C₂₋₆ alkynyl optionally substituted with OH; phenyl optionally substituted with OH, halo, hydroxyalkyl, sulfonyl, or alkylsulfonyl; pyrazolyl optionally substituted with C₁₋₆ alkyl, C₁₋₆ hydroxyalkyl, or acetamide; pyridinyl optionally substituted with amino; triazolyl optionally substituted with C₁₋₆ hydroxyalkyl; thiazolyl optionally substituted with C₁₋₆ alkyl; or 1,3,4-thiadiazolyl optionally substituted with C₁₋₆ alkyl;
n* is 0, 1 or 2;
R² is absent, halo, or C₁₋₆ alkyl, or cyano.

In some embodiments, M is

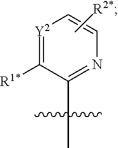

wherein
Y² is CH or N;
R^{1*} is absent; chloro; cyano; amido; alkylamido; allyl; alkoxycarbonyl; pyrazolyl; methylpyrazolyl; hydroxyethylpyrazolyl; isoxazolyl; 2,5-dihydro-pyrrolyl; thiophenyl; thieno[3,2-c]pyridinyl; C₁₋₆ alkyl; C₂₋₆ alkenyl optionally substituted with OH; or C₂₋₆ alkynyl optionally substituted with OH;
R^{2*} is absent; C₁₋₆ alkyl; halo; pyrazolyl; C₂₋₆ alkenyl optionally substituted with phenyl, methylpyrazolyl, or pyridinyl.

In some embodiments, M is

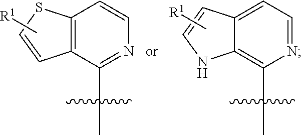

wherein
R¹ is absent, bromo, fluoro; chloro; amido; alkyl substituted amido; cyano; C₁₋₆ cycloalkyl; C₁₋₆ alkyl optionally substituted with phenyl or pyridinyl; C₂₋₆ alkenyl optionally substituted with OH, amido, alkyl substituted amido, or alkoxycarbonyl; C₂₋₆ alkynyl optionally substituted with OH, alkyl substituted amido, or pyridinyl; phenyl optionally substituted with one, two or three groups chosen from halo, OH, hydroxyalkyl, halohydroxyalkyl, cyano, CH₃O—, carboxyl, amino, amido, amidoalkyl, amidoalkoxy, alkyl substituted amido, phenyl substituted amido, hydroxyalkylamido, acetamido, aldehyde, acetyl, hydroxyalkylamido, sulfonylamino, alkylsulfonyl, isoxazolyl, pyrazolyl, and thiourea; pyridinyl optionally substituted with one or two groups chosen from C₁₋₆ alkyl, halo, amino, cyano, cyanoalkyl, haloalkyl, and alkoxy; 1,2,3-triazolyl optionally substituted with C₁₋₆ alkyl; thiazolyl optionally substituted with C₁₋₆ alkyl, hydroxyalkyl, alkoxycarbonyl, or hydroxycarbonyl; pyrazolyl optionally substituted with C₁₋₆ alkyl, hydroxyalkyl, pyridinyl, methylpyridinyl, or acetamido; benzo[d]isoxazolyl optionally substituted with amino; thiophenyl optionally substituted with hydroxyalkyl, cyano, or amido; pyrimidinyl optionally substituted with amino or alkylamino; pyridylcarbonyl; [1,2,4]triazolo[1,5-a]pyridinyl; pyrazinyl; pyridazinyl; pyridinyl-2-one; isoindolinyl-1-one; indolinyl; indolinyl-2-one; 1,3-dihydro-2H-benzo[d]imidazolyl-2-one; or indazolyl, wherein each substituted group has 1, 2 or 3 substituents.

In some embodiments, M is

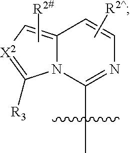

wherein
X² is CH, N, C-alkyl, C-alkenyl, C-alkynyl, C-aryl or C-heteroaryl;
R^{2#} is absent, halo, alkyl, alkenyl, alkynyl, aryl, ester, carboxylic acid, amido, alkoxy, thioether, amino, cyano, heteroaryl, cycloalkyl or heterocyclyl;
R^{2\hat{ }} is absent, alkyl, alkenyl, alkynyl, cyano or halo; and
R₃ is H, alkyl, alkenyl, alkynyl, halo, cyano, ester, carboxylic acid, amide, aryl, heteroaryl, alkoxy, thioether, or amino.

In some embodiments, L is a bond, phenylene, pyridinylene, piperidinylene, or piperazinylene, each optionally substituted with 1 or 2 substituents chosen from halo, C₁₋₃ alkyl, C₁₋₃ alkyloxy and combinations thereof, or —NR^{a}—, where R^{a} is H or C₁₋₃ alkyl. In some embodiments, L is a bond. In some embodiments, L is phenylene, pyridinylene, piperidinylene, or piperazinylene, each optionally substituted with 1 or 2 substituents chosen from halo, C₁₋₃ alkyl, C₁₋₃ alkyloxy and combinations thereof. In some embodiments, L is chosen from phenylene, fluorophenylene, chlorophenylene, methoxyphenylene, methylphenylene, ethylphenylene, pyridinylene, piperidinylene, and piperazinylene.

In some embodiments, Z is a bond. In some embodiments, Z is C₁₋₃ alkylene. In some embodiments, Z is methylene. In some embodiments, Z is —NH. In some embodiments, Z is —N(CH₃)—.

In some embodiments, T is a substituted or unsubstituted group chosen from [1,2,3]triazolo[4,5-b]pyridinyl, pyrazolo[2,3-a]pyrimidinyl, 5,6-dihydropyrrolo[2,3-d]pyrimidinyl, pyrazolo[3,4-d]pyrimidinyl, phenyl, purinyl, pyrimidinyl, pyridinyl, pyrazinyl, pyridazinyl, pyrazolyl, tetrazolyl, 1,2,4-triazolyl, 1,2,3-triazolyl, imidazolyl, benzo[d]imidazolyl, pyrrolyl, pyrrolidinyl, isoxazolyl, isoindolyl, piperidinyl, oxetanyl, thiophenyl, morpholino, thiazolyl, pyridinyl-2-one, pyrazinyl-2-one, indazolyl, 4,5,6,7-tetrahydro-indazolyl, isoindolinyl-1,3-dione, 1,3,4-oxadiazolyl, 1,3,4-oxadiazolyl-2-one, benzo[d]isoxazolyl, benzo[d]isothiazolyl, 1,2,4-oxadiazolyl, 1,3,4-thiadiazolyl, 1,3-dihydro-2H-imidazolyl-2-one, phthalazinyl, isoquinolinyl, 2λ⁵-quinolinyl-2-one, 6,7-dihydro-4H-[1,2,3]triazolo[5,1-c][1,4]oxazinyl, 2,3-dihydro-4λ²-pyrido[3,2-b][1,4]oxazinyl, thiazolo[5,4-c]pyridinyl, and quinazolinyl. In some embodiments T is not 5,5-dimethylisoxazolyl-4(5H)-one. In some embodiments, T is not a substituted 5,5-dimethylisoxazolyl-4(5H)-one. In some embodiments T is not 4-alkoxy-5,5-dimethyl-4,5-dihydroisoxazolyl. In some embodiments T is not a substituted 4-alkoxy-5,5-dimethyl-4,5-dihydroisoxazolyl.

In some embodiments, T is [1,2,3]triazolo[4,5-b]pyridinyl optionally substituted with halo, haloalkyl, cyano, methyl, methoxy, ethyl, pyrazolyl, alkoxycarbonyl, hydroxycarbonyl, amido, or phenyl; 5,6-dihydropyrrolo[2,3-d]pyrimidinyl optionally substituted with methyl; isoxazolyl optionally substituted with methyl, haloalkyl, alkyoxycarbonyl, or amido; thiazolyl optionally substituted with methyl; pyrazolyl optionally substituted with C₁₋₆ alkyl, C₁₋₆ hydroxyalkyl, C₁₋₆ alkoxyalkyl, amino, phenyl, hydroxycarbonyl or any combination of two or three thereof; pyrimidinyl optionally substituted with amido, amino, or one or two methyl groups; 1,2,3-triazolyl optionally substituted with C₁₋₆ alkyl, C₁₋₆ hydroxyalkyl, C₁₋₆ alkoxyalkyl, halo, or any combination of two or three thereof; 1,2,4-triazolyl optionally substituted with methyl; phenyl optionally substituted with OH, pyrazolyl, pyridinyl, cyano, indazolyl, amidoindazolyl, methoxy, benzyloxy, C₂₋₆ alkynyl, or any combination of two or three thereof, and wherein the C₂₋₆ alkynyl is further substituted by OH, oxo, alkoxy, isoindolinyl-1,3-dione, morpholino, amino, hydroxyalkyl substituted amino, pyrrolidinyl substituted amino, hydroxypyrrolidinyl substituted amino, pyridinyl substituted amino, pyridinylmethyl substituted amino, or any combination of two or three thereof; pyridinyl optionally substituted with halo, methyl, amino pyridinyl or any combination of two or three thereof; pyrazolo[3,4-d]pyrimidinyl optionally substituted with phenoxyphenyl; imidazolyl optionally substituted phenyl, methyl, amino, or any combination of two or three thereof; pyrazinyl optionally substituted with OH, halo, cyano, amino, substituted amino, C₁₋₆ alkoxycarbonyl, C₁₋₆ alkyl, C₁₋₆ hydroxyalkyl, C₁₋₆ alkoxyalkyl, C₁₋₈ cycloalkyl, substituted C₁₋₈ cycloalkyl, C₁₋₃ alkyl substituted with cycloalkyl, C₁₋₃ alkyl substituted with substituted cycloalkyl, heterocyclyl, substituted heterocyclyl, C₁₋₃ alkyl substituted with heterocyclyl, C₁₋₃ alkyl substituted with substituted heterocyclyl, heteroaryl, substituted heteroaryl, C₁₋₃ alkyl substituted with heteroaryl, C₁₋₃ alkyl substituted with substituted heteroaryl, aryl, substituted aryl, C₁₋₃ alkyl substituted with aryl, C₁₋₃ alkyl substituted with substituted aryl, C₂₋₆ alkonyl, C₂₋₆ hydroxyalkenyl, C₂₋₆ alkoxyalkenyl, C₂₋₆ alkynyl, C₂₋₆ hydroxyalkynyl, C₂₋₆ alkoxyalkynyl or any combination of two or three thereof; tetrazolyl optionally substituted with methyl, ethyl, or haloalkyl; 1,3,4-oxadiazolyl optionally substituted with C₁₋₆ alkyl, C₃₋₆ cycloalkyl; 1,3,4-thiadiazolyl optionally substituted with C₁₋₆ alkyl, C₁₋₆ hydroxyalkyl, C₁₋₆ alkoxyalkyl, C₃₋₆ cycloalkyl, CD₃, OH; pyridinyl-2-one optionally substituted with methyl; 1,3-dihydro-2H-imidazolyl-2-one optionally substituted with methyl; 1,3,4-oxadiazolyl-2-one optionally substituted with methyl; pyrrolidinyl optionally substituted with methyl; benzo[d]isothiazolyl optionally substituted with methyl; 1,2,4-oxadiazolyl optionally substituted with C₁₋₆ alkyl, cyclopropyl or phenyl; morpholino optionally substituted with 1 or 2 C₁₋₆ alkyl groups; or pyridazinyl optionally substituted with halo, wherein each substituted group has 1, 2 or 3 substituents. In some embodiments, T is not 5,5-dimethylisoxazolyl-4(5H)-one or 4-alkoxy-5,5-dimethyl-4,5-dihydroisoxazolyl.

In some embodiments, the * stereocenter is in the (R) configuration. In some embodiments, the * stereocenter is in the(S) configuration.

In some embodiments, the compound is of Formula (Ia), (Ib), (Ic), or a pharmaceutically acceptable salt thereof:

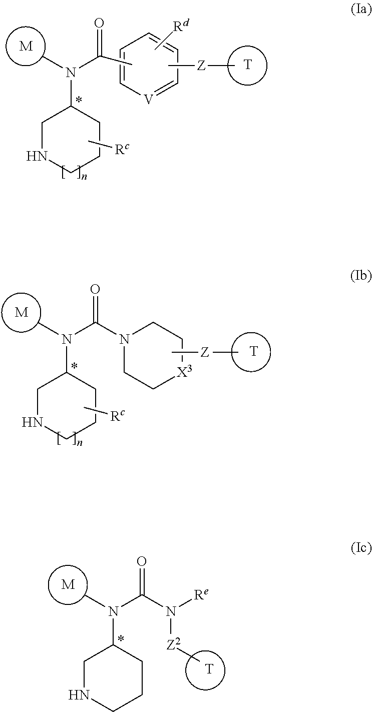

wherein
* denotes a stereocenter;
M is pyrrolo[2,3-c]pyridinyl, pyrrolo[3,2-c]pyridinyl, pyrazolo[3,4-c]pyridinyl, thieno[2,3-c]pyridinyl, thieno[3,2-c]pyridinyl, quinolinyl, isoquinolinyl, quinolinyl-2-one, 1,6-naphthyridinyl, 1,6-naphthyridinyl, pyridinyl, 2-pyrazinyl, thiazolo[5,4-c]pyridinyl, or thiazolyl, each optionally substituted with 1, 2 or 3 substituents independently chosen from halo, substituted or unsubstituted alkyl, substituted or unsubstituted haloalkyl, substituted or unsubstituted alkenyl, substituted or unsubstituted alkynyl, substituted or unsubstituted ester, carboxylic acid, substituted or unsubstituted acetyl, substituted or unsubstituted amido, substituted or unsubstituted carbamate, substituted or unsubstituted amino, substituted or unsubstituted alkoxy, substituted or unsubstituted alkoxycarbonyl, substituted or unsubstituted thioether, cyano, substituted or unsubstituted aryl, substituted or unsubstituted heteroaryl, substituted or unsubstituted cycloalkyl, substituted or unsubstituted heterocyclyl, and combinations thereof, wherein each substituted group has 1, 2 or 3 substituents;
V is N, or CH;
R^{d} is absent, halo, cyano, C₁₋₃ alkyl, or C₁₋₃ alkyloxy;
R^{c} is absent, H, C₁-C₆ alkyl, OH, NH₂, fluoro or gem-difluoro;
n is 0, 1 or 2;
X³ is CH₂ (or CH if bonded to T through Z), NH (or N if bonded to T through Z);
R^{e} is H or C₁₋₃ alkyl;
Z² is a bond or C₁₋₃ alkylene; and
Z is a bond, C₁₋₃ alkylene, —(CH₂)₂C(O)NH—, —N(R^{b})—, where R^{b} is H, or C₁₋₃ alkyl; and
T is [1,2,3]triazolo[4,5-b]pyridinyl, pyrazolo[2,3-a]pyrimidinyl, 5,6-dihydropyrrolo[2,3-d]pyrimidinyl, pyrazolo[3,4-d]pyrimidinyl, phenyl, purinyl, pyrimidinyl, pyridinyl, pyrazinyl, pyridazinyl, pyrazolyl, tetrazolyl, 1,2,4-triazolyl, 1,2,3-triazolyl, imidazolyl, benzo[d]imidazolyl, pyrrolyl, pyrrolidinyl, isoxazolyl, isoindolyl, piperidinyl, oxetanyl, thiophenyl, morpholino, thiazolyl, pyridinyl-2-one, pyrazinyl-2-one, indazolyl, 4,5,6,7-tetrahydro-indazolyl, isoindolinyl-1,3-dione, 1,3,4-oxadiazolyl, 1,3,4-oxadiazolyl-2-one, benzo[d]isoxazolyl, benzo[d]isothiazolyl, 1,2,4-oxadiazolyl, 1,3,4-thiadiazolyl, 1,3-dihydro-2H-imidazolyl-2-one, phthalazinyl, isoquinolinyl, 2λ⁵-quinolinyl-2-one, 6,7-dihydro-4H-[1,2,3]triazolo[5,1-c][1,4]oxazinyl, 2,3-dihydro-4λ²-pyrido[3,2-b][1,4]oxazinyl, thiazolo[5,4-c]pyridinyl, or quinazolinyl, each optionally substituted with 1, 2 or 3 substituents independently chosen from OH, halo, cyano, substituted or unsubstituted aryl, substituted or unsubstituted heteroaryl, substituted or unsubstituted alkyl, haloalkyl, substituted or unsubstituted alkenyl, substituted or unsubstituted alkynyl, substituted or unsubstituted cycloalkyl, substituted or unsubstituted heterocyclyl, substituted or unsubstituted alkoxycarbonyl, hydroxycarbonyl, substituted or unsubstituted ester, substituted or unsubstituted amido, substituted or unsubstituted alkoxy, substituted or unsubstituted alkoxyaryl, substituted or unsubstituted alkoxyheteroaryl, substituted or unsubstituted thioether, substituted or unsubstituted amino, NH-aryl, and NH-heteroaryl, wherein each substituted group has 1, 2 or 3 substituents;
provided that when the compound is of Formula (Ia) then
i) when n is 1, R^{c} is absent, R^{d} is absent, Z is a bond, and M is 3-chloropyridin-2-yl or 3-methylpyridin-2-yl, then T is not unsubstituted pyrazolo[2,3-a]pyrimidinyl; methyl substituted pyrazolo[2,3-a]pyrimidinyl; unsubstituted [1,2,3]triazolo[4,5-b]pyridinyl; methyl substituted [1,2,3]triazolo[4,5-b]pyridinyl; or pyrazolyl substituted with a methyl group, a substituted carboxyl group, a substituted or unsubstituted tetrazolyl group, or a substituted or unsubstituted 1,3,4-oxadiazolyl-2-one group; or
ii) when n is 1, R^{c} is absent, R^{d} is absent, Z is a bond, and M is unsubstituted isoquinolinyl or methyl substituted pyrrolo[2,3-c]pyridinyl, then T is not a pyrazolyl substituted with methyl, substituted carboxyl or both methyl and substituted carboxyl; or
iii) when M is phenyl or pyridinyl, and Z is a bond, then T is not 5,5-dimethylisoxazolyl-4(5H)-one or 4-alkoxy-5,5-dimethyl-4,5-dihydroisoxazolyl.

In some embodiments, R^{c} is absent, methyl, ethyl, OH, NH₂, or fluoro and Z is a bond, methylene, —NH— or —N(CH₃)—.

In some embodiments, when the compound is of Formula (I), (Ia), (Ib), (Ic), or a pharmaceutically acceptable salt thereof and Z is bond and T is pyrazinyl, the pyrazinyl groups is not further substituted with a substituted or unsubstituted group chosen from cycloalkyl, heterocycloalkyl, aryl and heteroaryl.

In some embodiments, provided herein is a compound of Formula Ia, and pharmaceutically acceptable salts thereof,

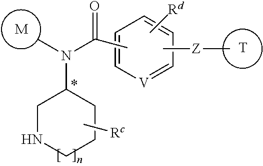

wherein
* denotes a stereocenter with (R) configuration;
wherein M is isoquinolinyl or a 9 membered fused bicyclic heteroaryl ring having 2 heteroatoms selected independently from nitrogen and sulfur, the isoquinolinyl or 9 membered ring optionally substituted with 1 to 2 substituents independently selected from pyridinyl, C₁₋₃ alkyl, C₁₋₃ alkoxy, fluoro or chloro; V is CH; n is 1; R^{c} is absent; R^{d} is absent, C₁₋₃ alkyl, C₁₋₃ alkoxy, fluoro or chloro; Z is a bond; and T is a 5 membered heteroaryl or a 9 membered fused bicyclic ring each 5 or 9 membered ring having 3-4 heteroatoms selected independently from nitrogen and sulfur and each 5 or 9 membered ring optionally substituted with 1 to 2 substituents selected independently from C₁₋₃ alkyl, C(D)₃, C₁₋₃ alkoxy, or fluoro.

In some embodiments, M is thieno[3,2-c]pyridinyl or isoquinolinyl substituted with 1 to 2 substituents independently selected from pyridinyl, methyl, fluoro or chloro; T is a 9 membered fused bicyclic ring having 4 nitrogens and is optionally substituted with 1 to 2 substituents selected independently from fluoro, C₁₋₃ alkyl, C(D)₃.

In some embodiments, M is thieno[3,2-c]pyridinyl or isoquinolinyl substituted with 1 to 2 substituents independently selected from pyridinyl, methyl fluoro, or chloro; and T is a 5 membered heteroaryl having 3 heteroatoms selected independently from nitrogen and sulfur optionally substituted with 1 substituent selected independently from fluoro, C₁₋₃ alkyl, C(D)₃.

In some embodiments, M is thieno[3,2-c]pyridin-4-yl or isoquinolin-1-yl substituted with 1 to 2 substituents independently selected from pyridinyl, methyl or chloro; n is 1; R^{c} is absent; V is CH, R^{d} is absent, fluoro or chloro; Z is a bond; and T is [1,2,3]triazolo[4,5-b]pyridin-3-yl, thieno[3,2-c]pyridinyl, 1,2,3-triazol-4-yl or 1,3,4-thiadiazol-2-yl optionally substituted with 1 substituent selected independently from methyl or C(D)₃.

In some embodiments, M is thieno[3,2-c]pyridin-4-yl substituted with pyridinyl, methyl or chloro; n is 1; R^{c} is absent; V is CH, R^{d} is absent, fluoro or chloro; Z is a bond; and T is [1,2,3]triazolo[4,5-b]pyridin-3-yl, thieno[3,2-c]pyridinyl, 1,2,3-triazol-4-yl or 1,3,4-thiadiazol-2-yl optionally substituted with 1 substituent selected independently from methyl or C(D)₃.

In some embodiments, M is thieno[3,2-c]pyridin-4-yl substituted with pyridinyl or methyl; n is 1; R^{c} is absent; V is CH, R^{d} is fluoro or chloro; Z is a bond; and T is [1,2,3]triazolo[4,5-b]pyridin-3-yl, thieno[3,2-c]pyridinyl, or 1,3,4-thiadiazol-2-yl optionally substituted with 1 substituent selected independently from methyl or C(D)₃.

In some embodiments, provided herein is a compound of Formula Ia

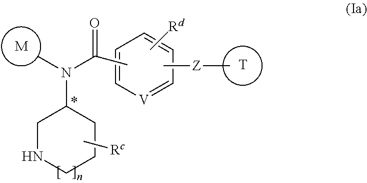

wherein
* denotes a stereocenter with (R) configuration;
M is isoquinolinyl substituted with 1 to 2 substituents independently selected from C₁₋₃ alkyl, C₁₋₃ alkoxy, fluoro or chloro; n is 1; R^{c} is absent; V is CH, R^{d} is absent, C₁₋₃ alkyl, C₁₋₃ alkoxy, fluoro or chloro; Z is a bond; and T is a 5 membered heteroaryl having 3 heteroatoms selected independently from nitrogen and sulfur optionally substituted with 1 to 2 substituents selected independently from C₁₋₃ alkyl, C(D)₃, C₁₋₃ alkoxy, or fluoro; and further wherein T is not a pyrazolyl substituted with methyl, substituted carboxyl or both methyl and substituted carboxyl.

In some embodiments, M is isoquinolinyl substituted with 1 to 2 substituents independently selected from methyl or chloro; n is 1; R^{c} is absent; V is CH, R^{d} is absent, methyl, fluoro or chloro; Z is a bond; and T is a 5 membered heteroaryl having 3 heteroatoms selected independently from nitrogen and sulfur optionally substituted with 1 substituent selected independently from methyl, C(D)₃, or fluoro.

In some embodiments, M is isoquinolin-1-yl substituted with 1 to 2 substituents independently selected from methyl or chloro; n is 1; R^{c} is absent; V is CH, R^{d} is absent, fluoro or chloro; Z is a bond; and T is 1,2,3-triazol-4-yl or 1,3,4-thiadiazol-2-yl optionally substituted with 1 substituent selected independently from methyl or C(D)₃.

In some embodiments, the compound is of (Ib), or a pharmaceutically acceptable salt thereof:

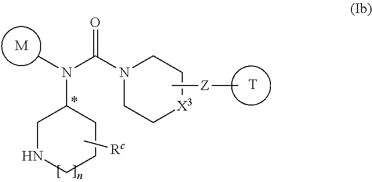

wherein
* denotes a stereocenter with (R) configuration;
X³ is CH₂ (or CH if bonded to T through Z), NH (or N if bonded to T through Z);
R^{c} is H, C₁-C₆ alkyl, OH, NH₂, fluoro or gem-difluoro;
n is 1; and
Z is a bond, C₁₋₃ alkylene, —(CH₂)₂C(O)NH—, —N(R^{b})—, where R^{b} is H, or C₁₋₃ alkyl.

In some embodiments, X is CH₂ (or CH if bonded to T through Z). In some embodiments, X is NH (or N if bonded to T through Z). In some embodiments, Z is Z is a bond, methylene, —(CH₂)₂C(O)NH—, —NH— or —N(CH₃)—.

In some embodiments, the compound is of Formula (Ic), or a pharmaceutically acceptable salt thereof:

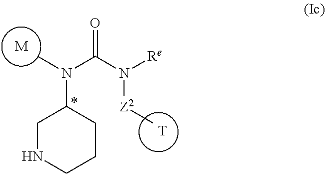

wherein
* denotes a stereocenter with (R) configuration;
R^{e} is H or C₁₋₃ alkyl; and
Z² is a bond or C₁₋₃ alkylene.

In some embodiments, R^{e} is H, methyl, or isopropyl. In some embodiments, Z² is a bond. In some embodiments, Z² is methylene.

In some embodiments, the compound is selected from those of Table 1. The chemical names used herein are generated using the MarvinSketch Version 6.1.6 (ChemAxon) or the ChemDraw Ultra Version 13.0 software naming programs.

TABLE 1

| Number | Name | Structure |
|---|---|---|
| 1 | (R)-N-(3-chloropyridin-2-yl)-4-(5-methyl- 3H-[1,2,3]triazolo[4,5-b]pyridin-3-yl)-N- (piperidin-3-yl)benzamide | 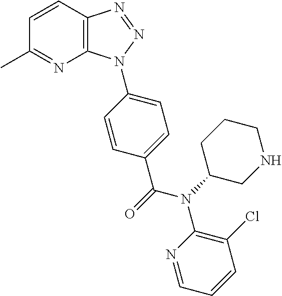 [SMILES: Cc1ccc2nnn(-c3ccc(C(=O)N(c4ncccc4Cl)[C@@H]4CCCNC4)cc3)c2n1] |
| 2 | (R)-N-(isoquinolin-1-yl)-N-(piperidin-3- yl)-4-(pyrazolo[1,5-a]pyrimidin-3- yl)benzamide | 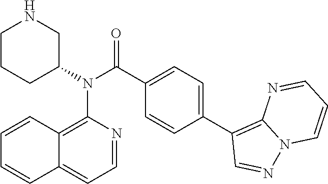 [SMILES: O=C(c1ccc(-c2cnn3cccnc23)cc1)N(c1nccc2ccccc12)[C@@H]1CCCNC1] |
| 3 | (R)-N-(1-methyl-1H-pyrrolo[2,3-c]pyridin- 7-yl)-N-(piperidin-3-yl)-4-(pyrazolo[1,5- alpyrimidin-3-yl)benzamide | 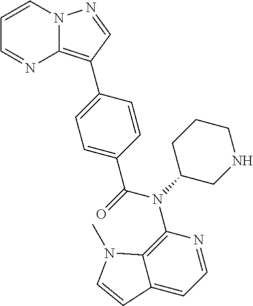 [SMILES: Cn1ccc2ccnc(N(C(=O)c3ccc(-c4cnn5cccnc45)cc3)[C@@H]3CCCNC3)c21] |
| 4 | (R)-4-(3-cyanophenyl)-N-(1-methyl-1H- pyrrolo[2,3-c]pyridin-7-yl)-N-(piperidin-3- yl)piperidine-1-carboxamide | 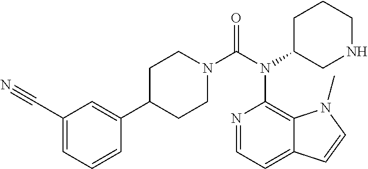 [SMILES: Cn1ccc2ccnc(N(C(=O)N3CCC(c4cccc(C#N)c4)CC3)[C@@H]3CCCNC3)c21] |
| 5 | (R)-N-(1-methyl-1H-pyrrolo[2,3-c]pyridin- 7-yl)-4-phenyl-N-(piperidin-3- yl)piperidine-1-carboxamide | 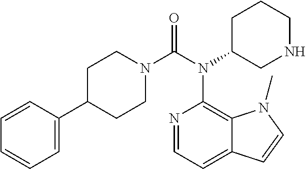 [SMILES: Cn1ccc2ccnc(N(C(=O)N3CCC(c4ccccc4)CC3)[C@@H]3CCCNC3)c21] |
| 6 | (R)-4-(3H-[1,2,3]triazolo[4,5-b]pyridin-3- yl)-N-(isoquinolin-1-yl)-N-(piperidin-3- yl)benzamide | 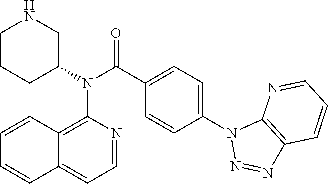 [SMILES: O=C(c1ccc(-n2nnc3cccnc32)cc1)N(c1nccc2ccccc12)[C@@H]1CCCNC1] |
| 7 | (R)-N-(3-chloropyridin-2-yl)-N-(piperidin- 3-yl)spiro[indole-3,4′-piperidine]-1′- carboxamide | 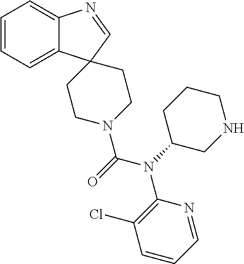 [SMILES: O=C(N1CCC2(C=Nc3ccccc32)CC1)N(c1ncccc1Cl)[C@@H]1CCCNC1] |
| 8 | (R)-1-(3-chloropyridin-2-yl)-3-(4- cyanobenzyl)-3-isopropyl-1-(piperidin-3- yl)urea | 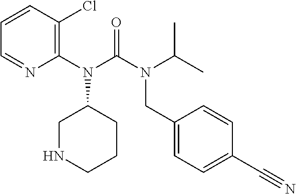 [SMILES: CC(C)N(Cc1ccc(C#N)cc1)C(=O)N(c1ncccc1Cl)[C@@H]1CCCNC1] |
| 9 | (R)-N-(3-chloropyridin-2-yl)-N-(piperidin- 3-yl)-4-(7H-purin-6-yl)piperazine-1- carboxamide | 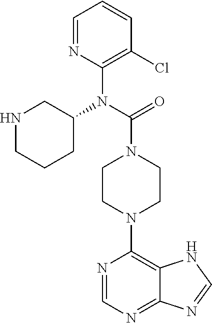 [SMILES: O=C(N1CCN(c2ncnc3nc[nH]c23)CC1)N(c1ncccc1Cl)[C@@H]1CCCNC1] |
| 10 | (R)-4-(1-methyl-1H-pyrazol-5-yl)-N-(1- methyl-1H-pyrrolo[2,3-c]pyridin-7-yl)-N- (piperidin-3-yl)piperidine-1-carboxamide | 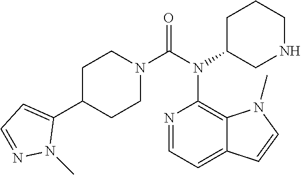 [SMILES: Cn1nccc1C1CCN(C(=O)N(c2nccc3ccn(C)c23)[C@@H]2CCCNC2)CC1] |
| 11 | (R)-4-(1-methyl-1H-pyrazol-4-yl)-N-(1- methyl-1H-pyrrolo[2,3-c]pyridin-7-yl)-N- (piperidin-3-yl)piperidine-1-carboxamide | 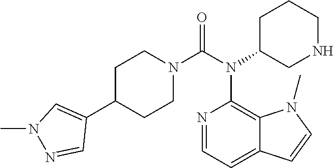 [SMILES: Cn1cc(C2CCN(C(=O)N(c3nccc4ccn(C)c34)[C@@H]3CCCNC3)CC2)cn1] |
| 12 | (R)-N-(1-methyl-1H-pyrrolo[2,3-c]pyridin- 7-yl)-N-(piperidin-3-yl)-4-(2H-tetrazol-2- yl)piperidine-1-carboxamide | 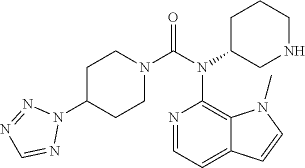 [SMILES: Cn1ccc2ccnc(N(C(=O)N3CCC(n4ncnn4)CC3)[C@@H]3CCCNC3)c21] |
| 13 | (R)-N-(1-methyl-1H-pyrrolo[2,3-c]pyridin- 7-yl)-N-(piperidin-3-yl)-4-(4H-1,2,4- triazol-4-yl)piperidine-1-carboxamide | 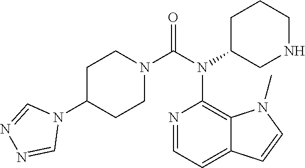 [SMILES: Cn1ccc2ccnc(N(C(=O)N3CCC(n4cnnc4)CC3)[C@@H]3CCCNC3)c21] |
| 14 | (R)-N-(1-methyl-1H-pyrrolo[2,3-c]pyridin- 7-yl)-N-(piperidin-3-yl)-4-(1H-1,2,3- triazol-1-yl)piperidine-1-carboxamide | 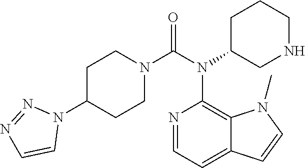 [SMILES: Cn1ccc2ccnc(N(C(=O)N3CCC(n4ccnn4)CC3)[C@@H]3CCCNC3)c21] |
| 15 | (R)-N-(1-methyl-1H-pyrrolo[2,3-c]pyridin- 7-yl)-N-(piperidin-3-yl)-4-(2H-1,2,3- triazol-2-yl)piperidine-1-carboxamide | 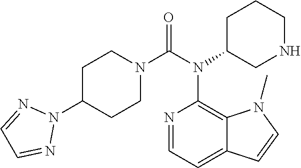 [SMILES: Cn1ccc2ccnc(N(C(=O)N3CCC(n4nccn4)CC3)[C@@H]3CCCNC3)c21] |
| 16 | (R)-4-cyano-N-(1-methyl-1H-pyrrolo[2,3- c]pyridin-7-yl)-4-phenyl-N-(piperidin-3- yl)piperidine-1-carboxamide | 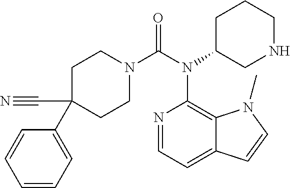 [SMILES: Cn1ccc2ccnc(N(C(=O)N3CCC(C#N)(c4ccccc4)CC3)[C@@H]3CCCNC3)c21] |
| 17 | (R)-4-(2-cyanophenyl)-N-(1-methyl-1H- pyrrolo[2,3-c]pyridin-7-yl)-N-(piperidin-3- yl)piperidine-1-carboxamide | 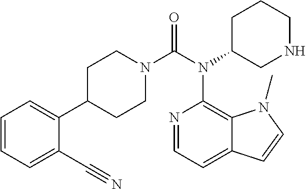 [SMILES: Cn1ccc2ccnc(N(C(=O)N3CCC(c4ccccc4C#N)CC3)[C@@H]3CCCNC3)c21] |
| 18 | (R)-1-(5-cyano-2-fluorobenzyl)-1-methyl- 3-(1-methyl-1H-pyrrolo[2,3-c]pyridin-7- yl)-3-(piperidin-3-yl)urea | 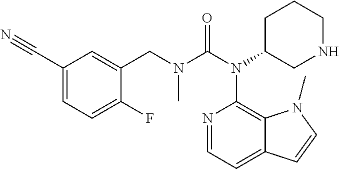 [SMILES: CN(Cc1cc(C#N)ccc1F)C(=O)N(c1nccc2ccn(C)c12)[C@@H]1CCCNC1] |
| 19 | (R)-1-(3-cyanobenzyl)-1-methyl-3-(1- methyl-1H-pyrrolo[2,3-c]pyridin-7-yl)-3- (piperidin-3-yl)urea | 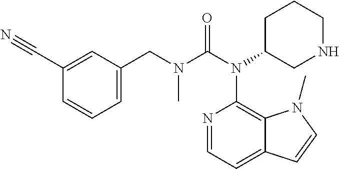 [SMILES: CN(Cc1cccc(C#N)c1)C(=O)N(c1nccc2ccn(C)c12)[C@@H]1CCCNC1] |
| 20 | (R)-1-(4-cyanobenzyl)-1-isopropyl-3-(1- methyl-1H-pyrrolo[2,3-c]pyridin-7-yl)-3- (piperidin-3-yl)urea | 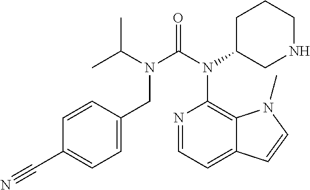 [SMILES: CC(C)N(Cc1ccc(C#N)cc1)C(=O)N(c1nccc2ccn(C)c12)[C@@H]1CCCNC1] |
| 21 | (R)-5-cyano-N-(1-methyl-1H-pyrrolo[2,3- c]pyridin-7-yl)-N-(piperidin-3- yl)isoindoline-2-carboxamide | 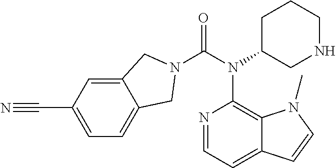 [SMILES: Cn1ccc2ccnc(N(C(=O)N3Cc4ccc(C#N)cc4C3)[C@@H]3CCCNC3)c21] |
| 22 | (R)-N-(1-methyl-1H-pyrrolo[2,3-c]pyridin- 7-yl)-N-(piperidin-3-yl)-4-(7H-purin-6- yl)piperazine-1-carboxamide | 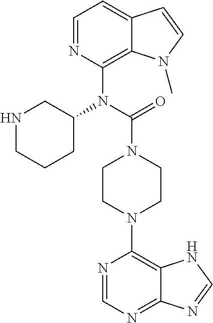 [SMILES: Cn1ccc2ccnc(N(C(=O)N3CCN(c4ncnc5nc[nH]c45)CC3)[C@@H]3CCCNC3)c21] |
| 23 | (R)-N-(1-methyl-1H-pyrrolo[2,3-c]pyridin- 7-yl)-N-(piperidin-3-yl)-4-(1H-pyrrolo[2,3- d]pyrimidin-4-yl)piperazine-1- carboxamide | 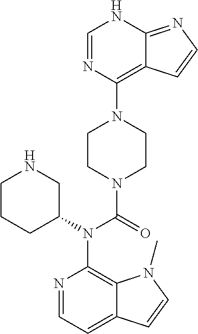 [SMILES: Cn1ccc2ccnc(N(C(=O)N3CCN(c4nc[nH]c5nccc4-5)CC3)[C@@H]3CCCNC3)c21] |
| 24 | (R)-4-(4-cyanopyrimidin-2-yl)-N-(1- methyl-1H-pyrrolo[2,3-c]pyridin-7-yl)-N- (piperidin-3-yl)piperazine-1-carboxamide | 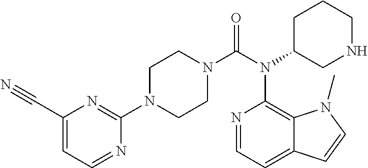 [SMILES: Cn1ccc2ccnc(N(C(=O)N3CCN(c4nccc(C#N)n4)CC3)[C@@H]3CCCNC3)c21] |
| 25 | (R)-4-(6-chloropyridin-2-yl)-N-(1-methyl- 1H-pyrrolo[2,3-c]pyridin-7-yl)-N- (piperidin-3-yl)piperazine-1-carboxamide | 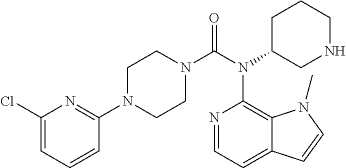 [SMILES: Cn1ccc2ccnc(N(C(=O)N3CCN(c4cccc(Cl)n4)CC3)[C@@H]3CCCNC3)c21] |
| 26 | (R)-4-(4-chloropyridin-2-yl)-N-(1-methyl- 1H-pyrrolo[2,3-c]pyridin-7-yl)-N- (piperidin-3-yl)piperazine-1-carboxamide | 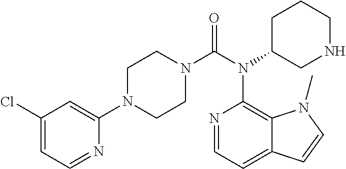 [SMILES: Cn1ccc2ccnc(N(C(=O)N3CCN(c4cc(Cl)ccn4)CC3)[C@@H]3CCCNC3)c21] |
| 27 | (R)-N-(1-methyl-1H-pyrrolo[2,3-c]pyridin- 7-yl)-N-(piperidin-3-yl)-4-(pyridin-2- yl)piperazine-1-carboxamide | 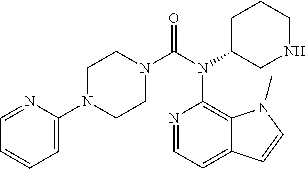 [SMILES: Cn1ccc2ccnc(N(C(=O)N3CCN(c4ccccn4)CC3)[C@@H]3CCCNC3)c21] |
| 28 | (R)-4′-cyano-N-(1-methyl-1H-pyrrolo[3,2- c]pyridin-6-yl)-N-(piperidin-3-yl)-[1,1′- biphenyl]-4-carboxamide | 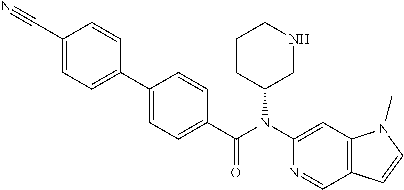 [SMILES: Cn1ccc2cnc(N(C(=O)c3ccc(-c4ccc(C#N)cc4)cc3)[C@@H]3CCCNC3)cc21] |
| 29 | (R)-4-(3H-[1,2,3]triazolo[4,5-b]pyridin-3- yl)-2-fluoro-N-(1-methyl-1H-pyrrolo[3,2- c]pyridin-6-yl)-N-(piperidin-3- yl)benzamide | 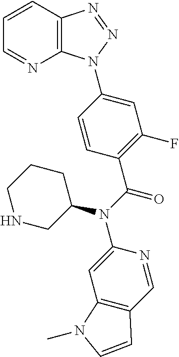 [SMILES: Cn1ccc2cnc(N(C(=O)c3ccc(-n4nnc5cccnc54)cc3F)[C@@H]3CCCNC3)cc21] |
| 30 | (R)-4′-cyano-N-(1-methyl-1H-pyrrolo[2,3- c]pyridin-5-yl)-N-(piperidin-3-yl)-[1,1′- biphenyl]-4-carboxamide | 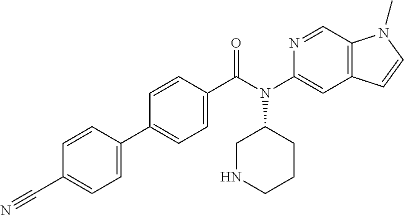 [SMILES: Cn1ccc2cc(N(C(=O)c3ccc(-c4ccc(C#N)cc4)cc3)[C@@H]3CCCNC3)ncc21] |
| 31 | (R)-4-(3H-[1,2,3]triazolo[4,5-b]pyridin-3- yl)-N-(1-methyl-1H-pyrrolo[2,3-c]pyridin- 5-yl)-N-(piperidin-3-yl)benzamide | 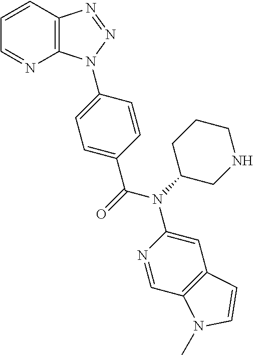 [SMILES: Cn1ccc2cc(N(C(=O)c3ccc(-n4nnc5cccnc54)cc3)[C@@H]3CCCNC3)ncc21] |
| 32 | (R)-4-(3H-[1,2,3]triazolo[4,5-b]pyridin-3- yl)-2-fluoro-N-(1-methyl-1H-pyrrolo[2,3- c]pyridin-5-yl)-N-(piperidin-3- yl)benzamide | 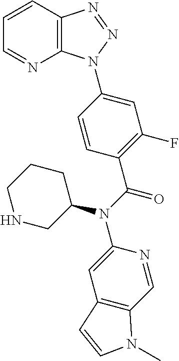 [SMILES: Cn1ccc2cc(N(C(=O)c3ccc(-n4nnc5cccnc54)cc3F)[C@@H]3CCCNC3)ncc21] |
| 33 | (R)-4-(3H-[1,2,3]triazolo[4,5-b]pyridin-3- yl)-2-methyl-N-(1-methyl-1H-pyrrolo[2,3- c]pyridin-5-yl)-N-(piperidin-3- yl)benzamide | 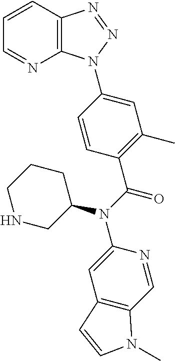 [SMILES: Cc1cc(-n2nnc3cccnc32)ccc1C(=O)N(c1cc2ccn(C)c2cn1)[C@@H]1CCCNC1] |
| 34 | (R)-4-(5-methyl-1H-pyrazol-1-yl)-N-(1- methyl-1H-pyrrolo[2,3-c]pyridin-5-yl)-N- (piperidin-3-yl)benzamide | 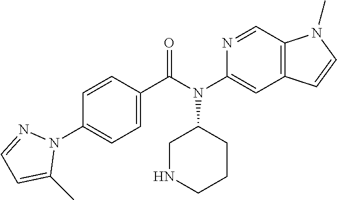 [SMILES: Cc1ccnn1-c1ccc(C(=O)N(c2cc3ccn(C)c3cn2)[C@@H]2CCCNC2)cc1] |
| 35 | (R)-4-(3H-[1,2,3]triazolo[4,5-b]pyridin-3- yl)-2-ethyl-N-(piperidin-3-yl)-N-(quinolin- 2-yl)benzamide | 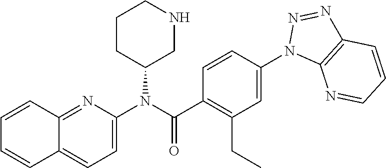 [SMILES: CCc1cc(-n2nnc3cccnc32)ccc1C(=O)N(c1ccc2ccccc2n1)[C@@H]1CCCNC1] |
| 36 | (R)-N-(piperidin-3-yl)-4-(pyridin-3- ylamino)-N-(quinolin-2-yl)benzamide | 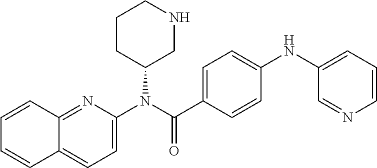 [SMILES: O=C(c1ccc(Nc2cccnc2)cc1)N(c1ccc2ccccc2n1)[C@@H]1CCCNC1] |
| 37 | (R)-4-(1H-imidazol-1-yl)-N-(piperidin-3- yl)-N-(quinolin-2-yl)benzamide | 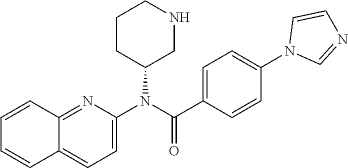 [SMILES: O=C(c1ccc(-n2ccnc2)cc1)N(c1ccc2ccccc2n1)[C@@H]1CCCNC1] |
| 38 | (R)-4-(3H-[1,2,3]triazolo[4,5-b]pyridin-3- yl)-2-chloro-N-(isoquinolin-3-yl)-N- (piperidin-3-yl)benzamide | 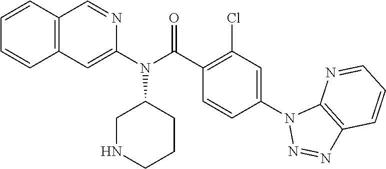 [SMILES: O=C(c1ccc(-n2nnc3cccnc32)cc1Cl)N(c1cc2ccccc2cn1)[C@@H]1CCCNC1] |
| 39 | (R)-4-(3H-[1,2,3]triazolo[4,5-b]pyridin-3- yl)-2-fluoro-N-(isoquinolin-3-yl)-N- (piperidin-3-yl)benzamide | 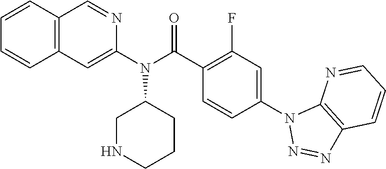 [SMILES: O=C(c1ccc(-n2nnc3cccnc32)cc1F)N(c1cc2ccccc2cn1)[C@@H]1CCCNC1] |
| 40 | (R)-4-(3H-[1,2,3]triazolo[4,5-b]pyridin-3- yl)-N-(3-methylpyridin-2-yl)-N-(piperidin- 3-yl)benzamide | 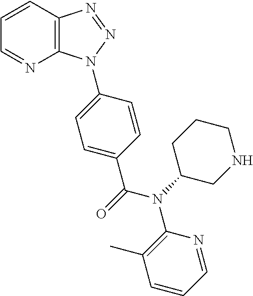 [SMILES: Cc1cccnc1N(C(=O)c1ccc(-n2nnc3cccnc32)cc1)[C@@H]1CCCNC1] |
| 41 | (R)-4-(3H-[1,2,3]triazolo[4,5-b]pyridin-3- yl)-2-chloro-N-(3-methylpyridin-2-yl)-N- (piperidin-3-yl)benzamide | 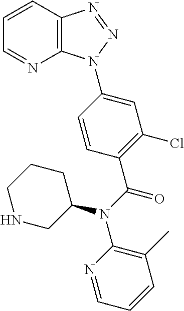 [SMILES: Cc1cccnc1N(C(=O)c1ccc(-n2nnc3cccnc32)cc1Cl)[C@@H]1CCCNC1] |
| 42 | (R)-4-(3H-[1,2,3]triazolo[4,5-b]pyridin-3- yl)-2-fluoro-N-(3-methylpyridin-2-yl)-N- (piperidin-3-yl)benzamide | 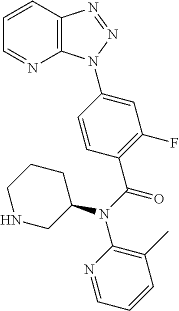 [SMILES: Cc1cccnc1N(C(=O)c1ccc(-n2nnc3cccnc32)cc1F)[C@@H]1CCCNC1] |
| 43 | (R)-4-(3H-[1,2,3]triazolo[4,5-b]pyridin-3- yl)-2-methyl-N-(3-methylpyridin-2-yl)-N- (piperidin-3-yl)benzamide | 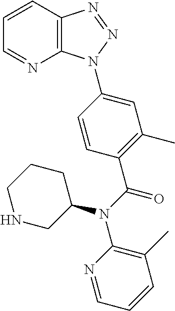 [SMILES: Cc1cc(-n2nnc3cccnc32)ccc1C(=O)N(c1ncccc1C)[C@@H]1CCCNC1] |
| 44 | (R)-4-(3H-[1,2,3]triazolo[4,5-b]pyridin-3- yl)-2-fluoro-N-(piperidin-3-yl)-N-(pyridin- 2-yl)benzamide | 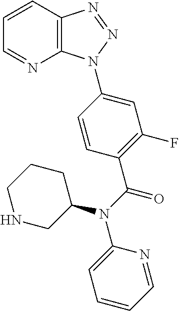 [SMILES: O=C(c1ccc(-n2nnc3cccnc32)cc1F)N(c1ccccn1)[C@@H]1CCCNC1] |
| 45 | (R)-4-(3H-[1,2,3]triazolo[4,5-b]pyridin-3- yl)-2-ethyl-N-(piperidin-3-yl)-N-(pyridin- 2-yl)benzamide | 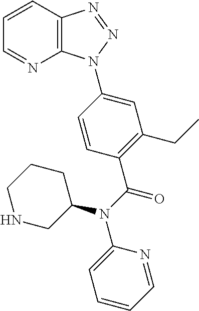 [SMILES: CCc1cc(-n2nnc3cccnc32)ccc1C(=O)N(c1ccccn1)[C@@H]1CCCNC1] |
| 46 | (R)-4-(3H-[1,2,3]triazolo[4,5-b]pyridin-3- yl)-2-chloro-N-(piperidin-3-yl)-N- (quinolin-2-yl)benzamide | 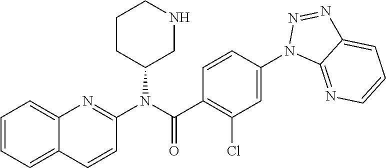 [SMILES: O=C(c1ccc(-n2nnc3cccnc32)cc1Cl)N(c1ccc2ccccc2n1)[C@@H]1CCCNC1] |
| 47 | (R)-4-(3H-[1,2,3]triazolo[4,5-b]pyridin-3- yl)-2-methyl-N-(8-methylisoquinolin-1- yl)-N-(piperidin-3-yl)benzamide | 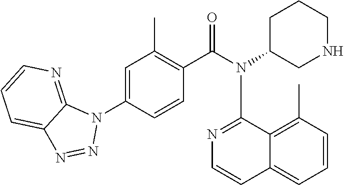 [SMILES: Cc1cc(-n2nnc3cccnc32)ccc1C(=O)N(c1nccc2cccc(C)c12)[C@@H]1CCCNC1] |
| 48 | (R)-4-(3H-[1,2,3]triazolo[4,5-b]pyridin-3- yl)-2-ethyl-N-(8-methylisoquinolin-1-yl)- N-(piperidin-3-yl)benzamide | 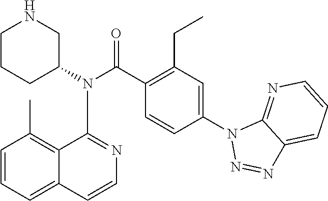 [SMILES: CCc1cc(-n2nnc3cccnc32)ccc1C(=O)N(c1nccc2cccc(C)c12)[C@@H]1CCCNC1] |
| 49 | (R)-4-(3H-[1,2,3]triazolo[4,5-b]pyridin-3- yl)-2-methoxy-N-(8-methylisoquinolin-1- yl)-N-(piperidin-3-yl)benzamide | 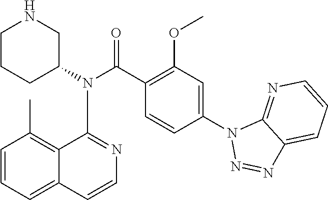 [SMILES: COc1cc(-n2nnc3cccnc32)ccc1C(=O)N(c1nccc2cccc(C)c12)[C@@H]1CCCNC1] |
| 50 | (R)-4-(3H-[1,2,3]triazolo[4,5-b]pyridin-3- yl)-N-(8-methylisoquinolin-1-yl)-N- (piperidin-3-yl)benzamide | 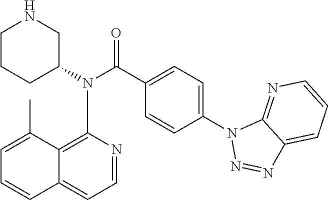 [SMILES: Cc1cccc2ccnc(N(C(=O)c3ccc(-n4nnc5cccnc54)cc3)[C@@H]3CCCNC3)c12] |
| 51 | (R)-N-(8-methylisoquinolin-1-yl)-N- (piperidin-3-yl)-4-(pyrimidin-2- ylamino)benzamide | 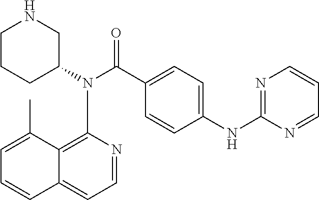 [SMILES: Cc1cccc2ccnc(N(C(=O)c3ccc(Nc4ncccn4)cc3)[C@@H]3CCCNC3)c12] |
| 52 | (R)-4-(3H-[1,2,3]triazolo[4,5-b]pyridin-3- yl)-2-fluoro-N-(3-methyl-3H- [1,2,3]triazolo[4,5-c]pyridin-4-yl)-N- (piperidin-3-yl)benzamide | 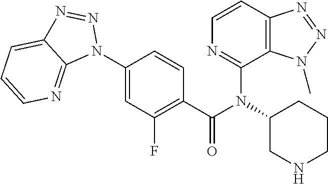 [SMILES: Cn1nnc2ccnc(N(C(=O)c3ccc(-n4nnc5cccnc54)cc3F)[C@@H]3CCCNC3)c21] |
| 53 | (R)-4-(3H-[1,2,3]triazolo[4,5-b]pyridin-3- yl)-2-chloro-N-(8-methylisoquinolin-1-yl)- N-(piperidin-3-yl)benzamide | 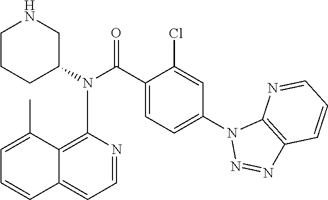 [SMILES: Cc1cccc2ccnc(N(C(=O)c3ccc(-n4nnc5cccnc54)cc3Cl)[C@@H]3CCCNC3)c12] |
| 54 | (R)-N-(8-methylisoquinolin-1-yl)-N- (piperidin-3-yl)-4-(1H-1,2,3-triazol-1- yl)benzamide | 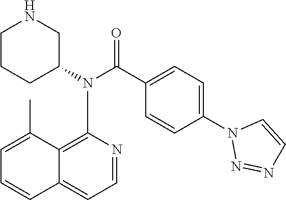 [SMILES: Cc1cccc2ccnc(N(C(=O)c3ccc(-n4ccnn4)cc3)[C@@H]3CCCNC3)c12] |
| 55 | (R)-4-(6-cyanopyrazin-2-yl)-N-(8- methylisoquinolin-1-yl)-N-(piperidin-3- yl)benzamide | 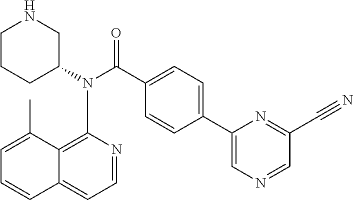 [SMILES: Cc1cccc2ccnc(N(C(=O)c3ccc(-c4cncc(C#N)n4)cc3)[C@@H]3CCCNC3)c12] |
| 56 | (R)-N-(8-methylisoquinolin-1-yl)-N- (piperidin-3-yl)-4-(pyrimidin-5- yl)benzamide | 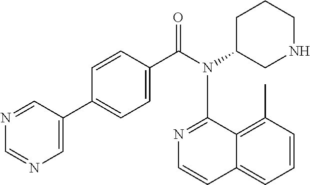 [SMILES: Cc1cccc2ccnc(N(C(=O)c3ccc(-c4cncnc4)cc3)[C@@H]3CCCNC3)c12] |
| 57 | (R)-4-(2-methyl-1H-benzo[d]imidazol-1- yl)-N-(8-methylisoquinolin-1-yl)-N- (piperidin-3-yl)benzamide | 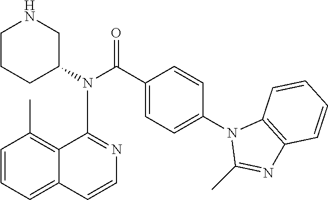 [SMILES: Cc1cccc2ccnc(N(C(=O)c3ccc(-n4c(C)nc5ccccc54)cc3)[C@@H]3CCCNC3)c12] |
| 58 | (R)-N-(3-methylpyrazin-2-yl)-N- (piperidin-3-yl)-4-(pyrimidin-2- yl)piperazine-1-carboxamide | 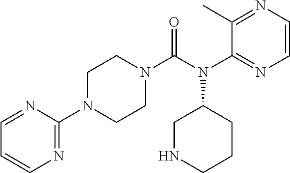 [SMILES: Cc1nccnc1N(C(=O)N1CCN(c2ncccn2)CC1)[C@@H]1CCCNC1] |
| 59 | (R)-N-(8-methylisoquinolin-1-yl)-N- (piperidin-3-yl)-4-(1H-pyrrol-1- yl)piperidine-1-carboxamide | 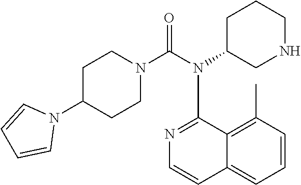 [SMILES: Cc1cccc2ccnc(N(C(=O)N3CCC(n4cccc4)CC3)[C@@H]3CCCNC3)c12] |
| 60 | (R)-4-(isoxazol-5-yl)-N-(8- methylisoquinolin-1-yl)-N-(piperidin-3- yl)piperidine-1-carboxamide | 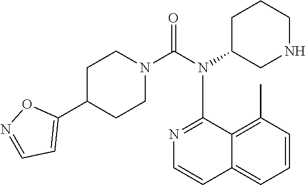 [SMILES: Cc1cccc2ccnc(N(C(=O)N3CCC(c4ccno4)CC3)[C@@H]3CCCNC3)c12] |
| 61 | (R)-N-(8-methylisoquinolin-1-yl)-4-(3- methylisoxazol-5-yl)-N-(piperidin-3- yl)piperidine-1-carboxamide | 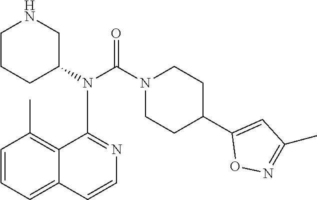 [SMILES: Cc1cc(C2CCN(C(=O)N(c3nccc4cccc(C)c34)[C@@H]3CCCNC3)CC2)on1] |
| 62 | (R)-N-(8-methylisoquinolin-1-yl)-N- (piperidin-3-yl)-4-(2H-tetrazol-2- yl)piperidine-1-carboxamide | 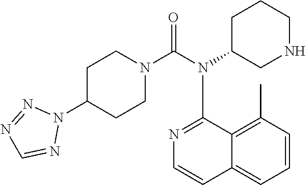 [SMILES: Cc1cccc2ccnc(N(C(=O)N3CCC(n4ncnn4)CC3)[C@@H]3CCCNC3)c12] |
| 63 | (R)-N-(8-methylisoquinolin-1-yl)-N- (piperidin-3-yl)-4-(1H-1,2,3-triazol-1- yl)piperidine-1-carboxamide | 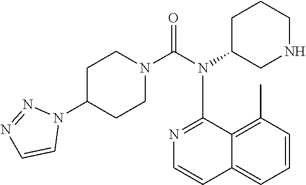 [SMILES: Cc1cccc2ccnc(N(C(=O)N3CCC(n4ccnn4)CC3)[C@@H]3CCCNC3)c12] |
| 64 | (R)-N-(1-methyl-1H-pyrrolo[2,3-c]pyridin- 5-yl)-N-(piperidin-3-yl)-4-(pyridin-4- ylamino)benzamide | 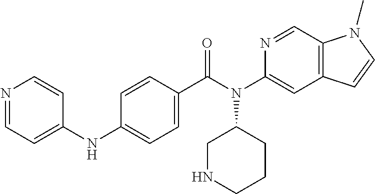 [SMILES: Cn1ccc2cc(N(C(=O)c3ccc(Nc4ccncc4)cc3)[C@@H]3CCCNC3)ncc21] |
| 65 | (R)-N-(piperidin-3-yl)-4-(pyridin-4- ylamino)-N-(quinolin-2-yl)benzamide | 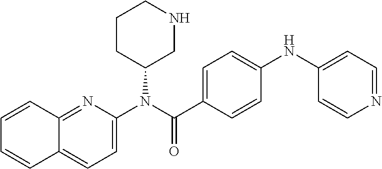 [SMILES: O=C(c1ccc(Nc2ccncc2)cc1)N(c1ccc2ccccc2n1)[C@@H]1CCCNC1] |
| 66 | (R)-N-(isoquinolin-3-yl)-N-(piperidin-3- yl)-4-(pyridin-3-ylamino)benzamide | 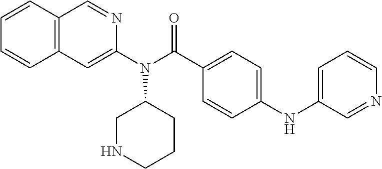 [SMILES: O=C(c1ccc(Nc2cccnc2)cc1)N(c1cc2ccccc2cn1)[C@@H]1CCCNC1] |
| 67 | (R)-N-(isoquinolin-3-yl)-N-(piperidin-3- yl)-4-(pyridin-4-ylamino)benzamide | 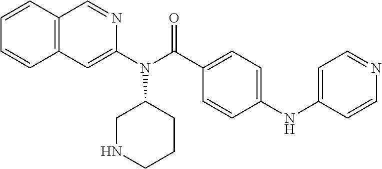 [SMILES: O=C(c1ccc(Nc2ccncc2)cc1)N(c1cc2ccccc2cn1)[C@@H]1CCCNC1] |
| 68 | (R)-N-(isoquinolin-3-yl)-4′-methoxy-N- (piperidin-3-yl)-[1,1′-biphenyl]-4- carboxamide | 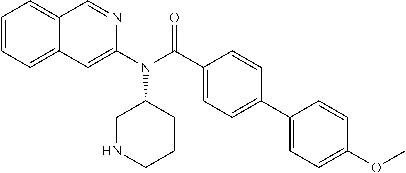 [SMILES: COc1ccc(-c2ccc(C(=O)N(c3cc4ccccc4cn3)[C@@H]3CCCNC3)cc2)cc1] |
| 69 | (R)-3-fluoro-N-(isoquinolin-3-yl)-4'- methoxy-N-(piperidin-3-yl)-[1,1′- biphenyl]-4-carboxamide | 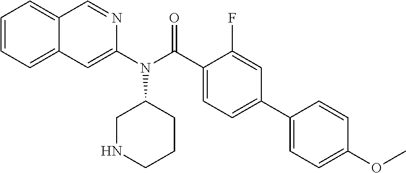 [SMILES: COc1ccc(-c2ccc(C(=O)N(c3cc4ccccc4cn3)[C@@H]3CCCNC3)c(F)c2)cc1] |
| 70 | (R)-2-fluoro-N-(isoquinolin-3-yl)-4-(1- methyl-1H-pyrazol-4-yl)-N-(piperidin-3- yl)benzamide | 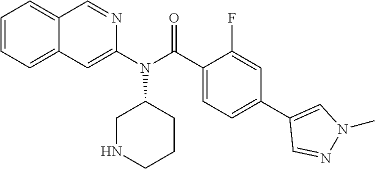 [SMILES: Cn1cc(-c2ccc(C(=O)N(c3cc4ccccc4cn3)[C@@H]3CCCNC3)c(F)c2)cn1] |
| 71 | (R)-2-fluoro-N-(isoquinolin-3-yl)-N- (piperidin-3-yl)-4-(pyridin-2-yl)benzamide | 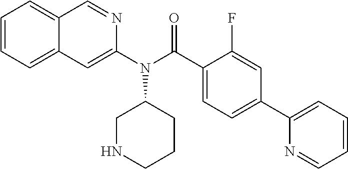 [SMILES: O=C(c1ccc(-c2ccccn2)cc1F)N(c1cc2ccccc2cn1)[C@@H]1CCCNC1] |
| 72 | (R)-2-fluoro-N-(isoquinolin-3-yl)-N- (piperidin-3-yl)-4-(pyridin-3-yl)benzamide | 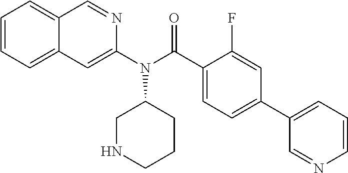 [SMILES: O=C(c1ccc(-c2cccnc2)cc1F)N(c1cc2ccccc2cn1)[C@@H]1CCCNC1] |
| 73 | (R)-2-fluoro-N-(isoquinolin-3-yl)-N- (piperidin-3-yl)-4-(pyridin-4-yl)benzamide | 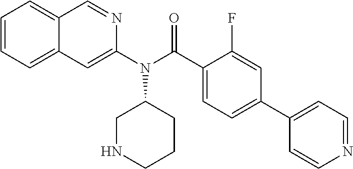 [SMILES: O=C(c1ccc(-c2ccncc2)cc1F)N(c1cc2ccccc2cn1)[C@@H]1CCCNC1] |
| 74 | (R)-N-(isoquinolin-3-yl)-N-(piperidin-3- yl)-[3,4′-bipyridine]-6-carboxamide | 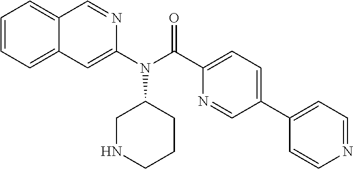 [SMILES: O=C(c1ccc(-c2ccncc2)cn1)N(c1cc2ccccc2cn1)[C@@H]1CCCNC1] |
| 75 | (R)-N-(isoquinolin-3-yl)-N-(piperidin-3- yl)-3-((pyridin-3- ylmethyl)amino)benzamide | 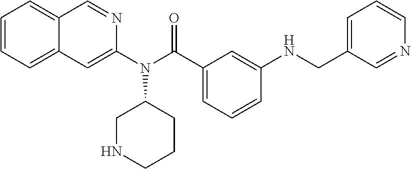 [SMILES: O=C(c1cccc(NCc2cccnc2)c1)N(c1cc2ccccc2cn1)[C@@H]1CCCNC1] |
| 76 | (R)-N-(1-methyl-1H-pyrrolo[2,3-c]pyridin- 5-yl)-N-(piperidin-3-yl)-3-(pyridin-4- yl)benzamide | 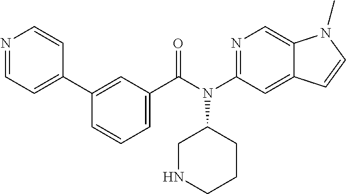 [SMILES: Cn1ccc2cc(N(C(=O)c3cccc(-c4ccncc4)c3)[C@@H]3CCCNC3)ncc21] |
| 77 | (R)-4-(1-methyl-1H-pyrazol-4-yl)-N-(3- methylpyridin-2-yl)-N-(piperidin-3- yl)benzamide | 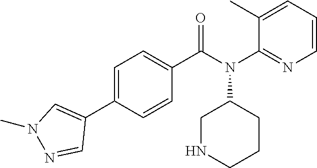 |
| 78 | (R)-N-(3-methylpyridin-2-yl)-N- (piperidin-3-yl)-4-(pyridin-2-yl)benzamide | 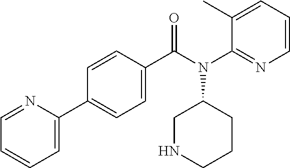 [SMILES: Cc1cccnc1N(C(=O)c1ccc(-c2ccccn2)cc1)[C@@H]1CCCNC1] |
| 79 | (R)-N-(3-methylpyridin-2-yl)-N- (piperidin-3-yl)-4-(pyridin-4-yl)benzamide | 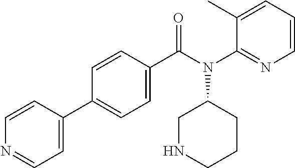 [SMILES: Cc1cccnc1N(C(=O)c1ccc(-c2ccncc2)cc1)[C@@H]1CCCNC1] |
| 80 | (R)-4-((1-methylpiperidin-4-yl)amino)-N- (3-methylpyridin-2-yl)-N-(piperidin-3- yl)benzamide | 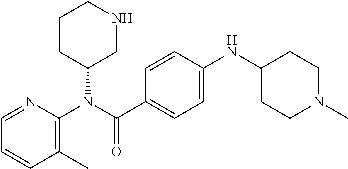 [SMILES: Cc1cccnc1N(C(=O)c1ccc(NC2CCN(C)CC2)cc1)[C@@H]1CCCNC1] |
| 81 | (R)-3-(1-methyl-1H-pyrazol-4-yl)-N-(3- methylpyridin-2-yl)-N-(piperidin-3- yl)benzamide | 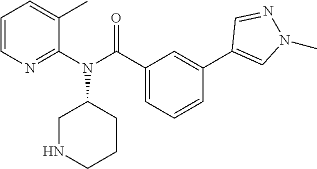 [SMILES: Cc1cccnc1N(C(=O)c1cccc(-c2cnn(C)c2)c1)[C@@H]1CCCNC1] |
| 82 | (R)-N-(3-methylpyridin-2-yl)-N- (piperidin-3-yl)-3-(pyridin-3-yl)benzamide | 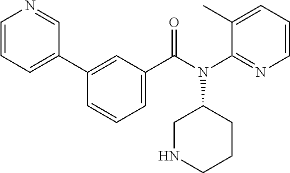 [SMILES: Cc1cccnc1N(C(=O)c1cccc(-c2cccnc2)c1)[C@@H]1CCCNC1] |
| 83 | (R)-4′-methoxy-N-(1-methyl-1H- pyrrolo[2,3-c]pyridin-5-yl)-N-(piperidin-3- yl)-[1,1'-biphenyl]-4-carboxamide | 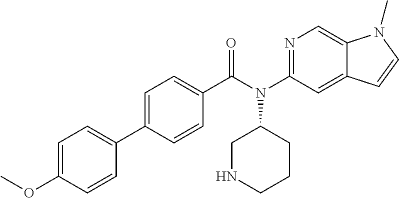 [SMILES: COc1ccc(-c2ccc(C(=O)N(c3cc4ccn(C)c4cn3)[C@@H]3CCCNC3)cc2)cc1] |
| 84 | (R)-N-(piperidin-3-yl)-N-(quinolin-2-yl)- [2,3′-bipyridine]-6'-carboxamide | 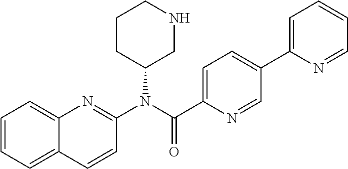 [SMILES: O=C(c1ccc(-c2ccccn2)cn1)N(c1ccc2ccccc2n1)[C@@H]1CCCNC1] |
| 85 | (R)-4′-methoxy-N-(piperidin-3-yl)-N- (quinolin-2-yl)-[1,1'-biphenyl]-3- carboxamide | 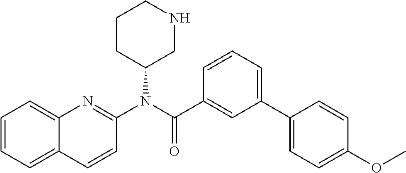 [SMILES: COc1ccc(-c2cccc(C(=O)N(c3ccc4ccccc4n3)[C@@H]3CCCNC3)c2)cc1] |
| 86 | (R)-3-fluoro-4′-methoxy-N-(1-methyl-1H- pyrrolo[2,3-c]pyridin-5-yl)-N-(piperidin-3- yl)-[1,1′-biphenyl]-4-carboxamide | 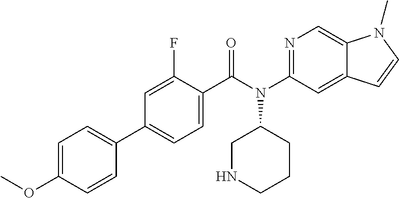 [SMILES: COc1ccc(-c2ccc(C(=O)N(c3cc4ccn(C)c4cn3)[C@@H]3CCCNC3)c(F)c2)cc1] |
| 87 | (R)-2-fluoro-4-(1-methyl-1H-pyrazol-4- yl)-N-(1-methyl-1H-pyrrolo[2,3-c]pyridin- 5-yl)-N-(piperidin-3-yl)benzamide | 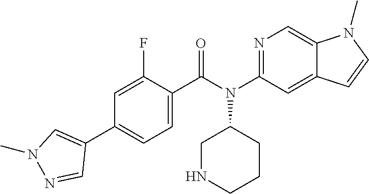 [SMILES: Cn1cc(-c2ccc(C(=O)N(c3cc4ccn(C)c4cn3)[C@@H]3CCCNC3)c(F)c2)cn1] |
| 88 | (R)-2-fluoro-N-(1-methyl-1H-pyrrolo[2,3- c]pyridin-5-yl)-N-(piperidin-3-yl)-4- (pyridin-3-yl)benzamide | 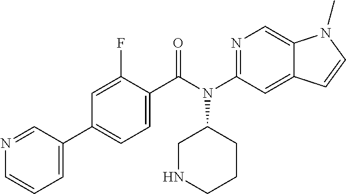 [SMILES: Cn1ccc2cc(N(C(=O)c3ccc(-c4cccnc4)cc3F)[C@@H]3CCCNC3)ncc21] |
| 89 | (R)-2-fluoro-N-(isoquinolin-3-yl)-4- (methyl(pyridin-3-yl)amino)-N-(piperidin- 3-yl)benzamide | 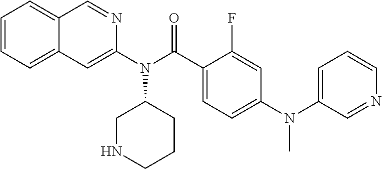 [SMILES: CN(c1cccnc1)c1ccc(C(=O)N(c2cc3ccccc3cn2)[C@@H]2CCCNC2)c(F)c1] |
| 90 | (R)-N-(3-methylpyridin-2-yl)-4-(oxetan-3- ylamino)-N-(piperidin-3-yl)benzamide | 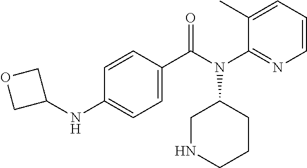 [SMILES: Cc1cccnc1N(C(=O)c1ccc(NC2COC2)cc1)[C@@H]1CCCNC1] |
| 91 | (R)-4-((1-methylpiperidin-4-yl)amino)-N- (piperidin-3-yl)-N-(quinolin-2- yl)benzamide | 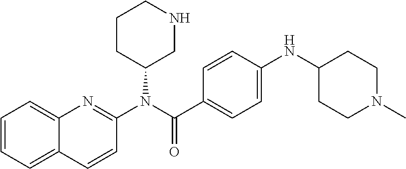 [SMILES: CN1CCC(Nc2ccc(C(=O)N(c3ccc4ccccc4n3)[C@@H]3CCCNC3)cc2)CC1] |
| 92 | (R)-2-fluoro-N-(piperidin-3-yl)-4-(pyridin- 3-ylamino)-N-(quinolin-2-yl)benzamide | 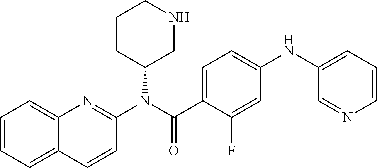 [SMILES: O=C(c1ccc(Nc2cccnc2)cc1F)N(c1ccc2ccccc2n1)[C@@H]1CCCNC1] |
| 93 | (R)-2-fluoro-N-(1-methyl-1H-pyrrolo[2,3- c]pyridin-5-yl)-N-(piperidin-3-yl)-4- (pyridin-4-yl)benzamide | 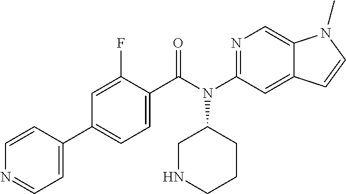 [SMILES: Cn1ccc2cc(N(C(=O)c3ccc(-c4ccncc4)cc3F)[C@@H]3CCCNC3)ncc21] |
| 94 | (R)-N-(isoquinolin-3-yl)-N-(piperidin-3- yl)-4-(pyridin-2-ylamino)benzamide | 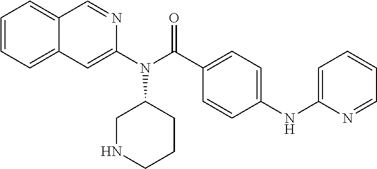 [SMILES: O=C(c1ccc(Nc2ccccn2)cc1)N(c1cc2ccccc2cn1)[C@@H]1CCCNC1] |
| 95 | (R)-N-(1-methyl-1H-pyrrolo[2,3-c]pyridin- 5-yl)-N-(piperidin-3-yl)-4-(pyridin-2- ylamino)benzamide | 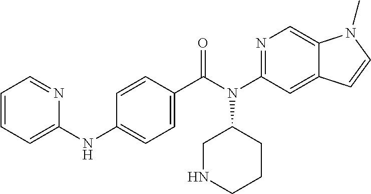 [SMILES: Cn1ccc2cc(N(C(=O)c3ccc(Nc4ccccn4)cc3)[C@@H]3CCCNC3)ncc21] |
| 96 | (R)-2-hydroxy-N-(1-methyl-1H- pyrrolo[2,3-c]pyridin-5-yl)-N-(piperidin-3- yl)-4-(pyrimidin-5-ylamino)benzamide | 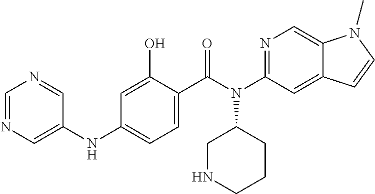 [SMILES: Cn1ccc2cc(N(C(=O)c3ccc(Nc4cncnc4)cc3O)[C@@H]3CCCNC3)ncc21] |
| 97 | (R)-4-(3H-[1,2,3]triazolo[4,5-b]pyridin-3- yl)-2-fluoro-N-(6-(1-methyl-1H-pyrazol-4- yl)isoquinolin-1-yl)-N-(piperidin-3- yl)benzamide | 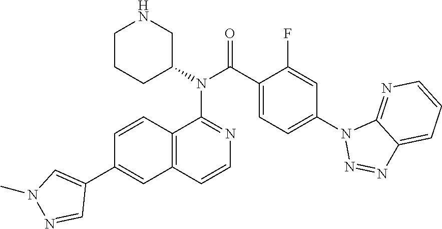 [SMILES: Cn1cc(-c2ccc3c(N(C(=O)c4ccc(-n5nnc6cccnc65)cc4F)[C@@H]4CCCNC4)nccc3c2)cn1] |
| 98 | ethyl (R)-3-(1-(4-(3H-[1,2,3]triazolo[4,5- b]pyridin-3-yl)-2-fluoro-N-(piperidin-3- yl)benzamido)isoquinolin-6-yl)acrylate | 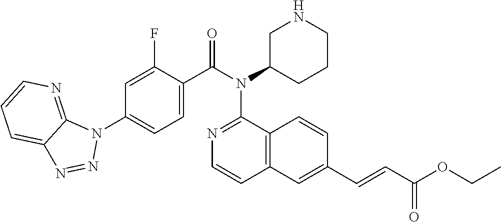 [SMILES: CCOC(=O)/C=C/c1ccc2c(N(C(=O)c3ccc(-n4nnc5cccnc54)cc3F)[C@@H]3CCCNC3)nccc2c1] |
| 99 | (R)-4-(3H-[1,2,3]triazolo[4,5-b]pyridin-3- yl)-N-(2-(3-((tert- butyldimethylsilyl)oxy)prop-1-en-1- yl)thieno[3,2-c]pyridin-4-yl)-2-fluoro-N- (piperidin-3-yl)benzamide | 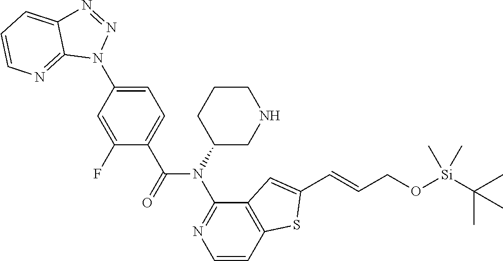 [SMILES: CC(C)(C)[Si](C)(C)OC/C=C/c1cc2c(N(C(=O)c3ccc(-n4nnc5cccnc54)cc3F)[C@@H]3CCCNC3)nccc2s1] |
| 100 | ethyl (R)-3-(1-(4-(1-methyl-1H-1,2,3- triazol-4-yl)-N-(piperidin-3- yl)benzamido)isoquinolin-8-yl)acrylate | 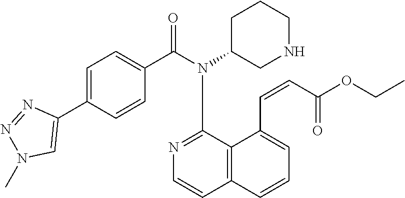 [SMILES: CCOC(=O)/C=C\c1cccc2ccnc(N(C(=O)c3ccc(-c4cn(C)nn4)cc3)[C@@H]3CCCNC3)c12] |
| 101 | (R)-N-(3-(1H-pyrazol-3-yl)pyridin-2-yl)-4- (1-methyl-1H-1,2,3-triazol-4-yl)-N- (piperidin-3-yl)benzamide | 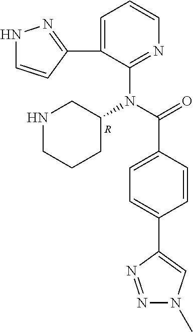 |
| 102 | (R)-4-(3H-[1,2,3]triazolo[4,5-b]pyridin-3- yl)-2-fluoro-N-(piperidin-3-yl)-N-(8- vinylisoquinolin-1-yl)benzamide | 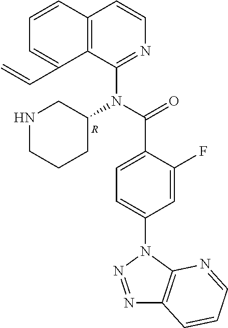 |
| 103 | (R)-4-(3H-[1,2,3]triazolo[4,5-b]pyridin-3- yl)-2-fluoro-N-(7-(3-hydroxyprop-1-yn-1- yl)isoquinolin-1-yl)-N-(piperidin-3- yl)benzamide | 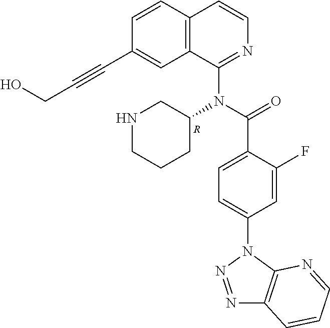 |
| 104 | (R)-4-(3H-[1,2,3]triazolo[4,5-b]pyridin-3- yl)-2-fluoro-N-(3-(4-hydroxybut-1-en-1- yl)pyridin-2-yl)-N-(piperidin-3- yl)benzamide | 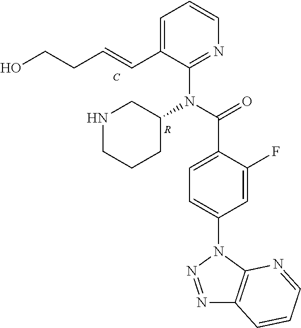 |
| 105 | (R)-4-(3H-[1,2,3]triazolo[4,5-b]pyridin-3- yl)-2-fluoro-N-(3-(4-hydroxybut-1-yn-1- yl)pyridin-2-yl)-N-(piperidin-3- yl)benzamide | 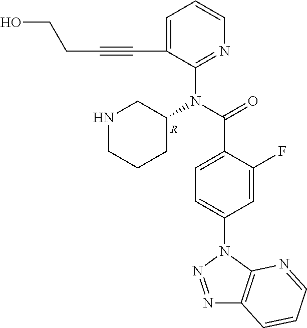 |
| 106 | (R)-N-(6-(1H-pyrazol-4-yl)isoquinolin-1- yl)-4-(3H-[1,2,3]triazolo[4,5-b]pyridin-3- yl)-2-fluoro-N-(piperidin-3-yl)benzamide | 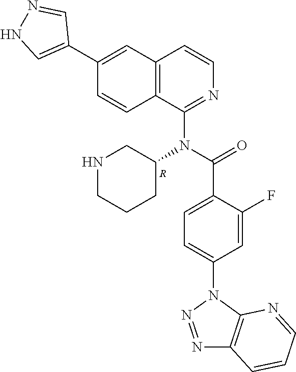 |
| 107 | (R)-4-(3H-[1,2,3]triazolo[4,5-b]pyridin-3- yl)-N-(3-ethynylpyridin-2-yl)-2-fluoro-N- (piperidin-3-yl)benzamide | 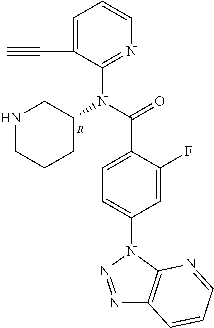 |
| 108 | (R)-4-(3H-[1,2,3]triazolo[4,5-b]pyridin-3- yl)-2-fluoro-N-(piperidin-3-yl)-N-(3-(prop- 1-en-2-yl)pyridin-2-yl)benzamide | 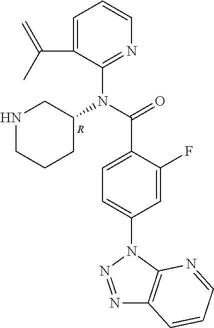 |
| 109 | (R)-4-(1-methyl-1H-1,2,3-triazol-4-yl)-N- (piperidin-3-yl)-N-(4-styrylpyridin-2- yl)benzamide | 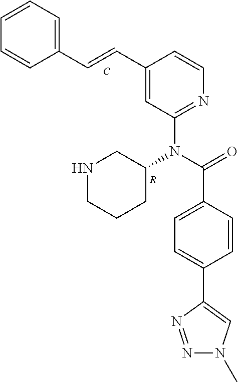 |
| 110 | (R)-N-(4-(1H-pyrazol-3-yl)pyridin-2-yl)-4- (1-methyl-1H-1,2,3-triazol-4-yl)-N- (piperidin-3-yl)benzamide | 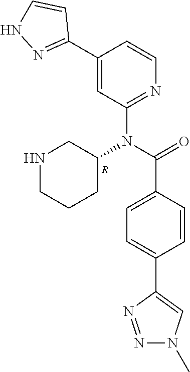 |
| 111 | (R)-4-(1-methyl-1H-1,2,3-triazol-4-yl)-N- (8-methylisoquinolin-1-yl)-N-(piperidin-3- yl)benzamide | 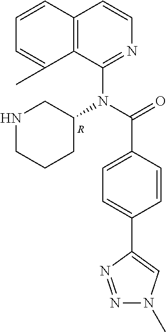 |
| 112 | (R)-4-(3H-[1,2,3]triazolo[4,5-b]pyridin-3- yl)-2-fluoro-N-(3-(1-methyl-1H-imidazol- 4-yl)pyridin-2-yl)-N-(piperidin-3- yl)benzamide | 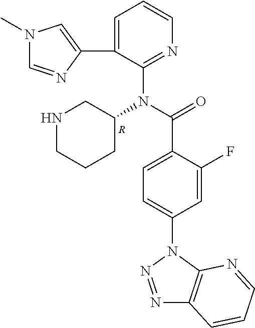 |
| 113 | (R)-4-(3H-[1,2,3]triazolo[4,5-b]pyridin-3- yl)-N-(3-(2,5-dihydro-1H-pyrrol-3- yl)pyridin-2-yl)-2-fluoro-N-(piperidin-3- yl)benzamide | 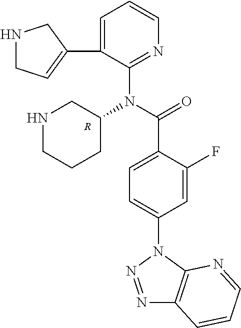 |
| 114 | (R)-4-(3H-[1,2,3]triazolo[4,5-b]pyridin-3- yl)-2-fluoro-N-(3-(isoxazol-4-yl)pyridin-2- yl)-N-(piperidin-3-yl)benzamide | 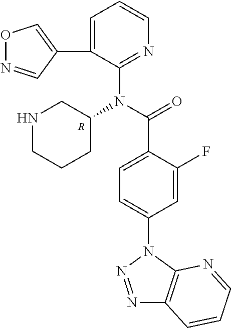 |
| 115 | (R)-4-(3H-[1,2,3]triazolo[4,5-b]pyridin-3- yl)-2-fluoro-N-(3-(1-methyl-1H-pyrazol-4- yl)pyridin-2-yl)-N-(piperidin-3- yl)benzamide | 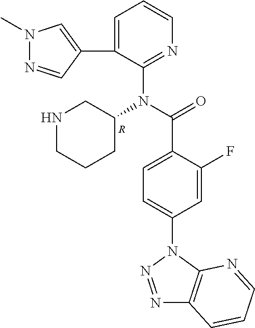 |
| 116 | (R)-4-(3H-[1,2,3]triazolo[4,5-b]pyridin-3- yl)-2-fluoro-N-(3-(1-(2-hydroxyethyl)-1H- pyrazol-4-yl)pyridin-2-yl)-N-(piperidin-3- yl)benzamide | 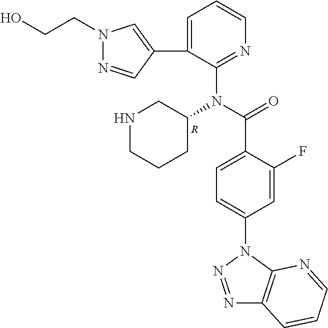 |
| 117 | methyl (R)-1-(4-(3H-[1,2,3]triazolo[4,5- b]pyridin-3-yl)-2-fluoro-N-(piperidin-3- yl)benzamido)isoquinoline-6-carboxylate | 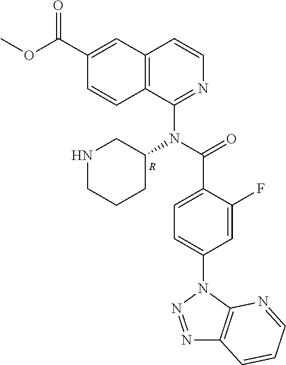 |
| 118 | (R)-1-(4-(3H-[1,2,3]triazolo[4,5-b]pyridin- 3-yl)-2-fluoro-N-(piperidin-3- yl)benzamido)isoquinoline-6-carboxylic acid | 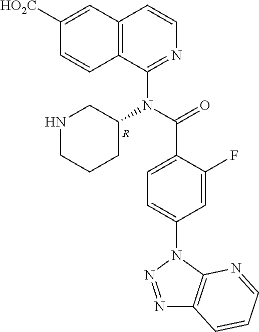 |
| 119 | (R)-2-fluoro-N-(1-methyl-1H- pyrazolo[3,4-c]pyridin-7-yl)-N-(piperidin- 3-yl)-4-(pyrimidin-2-ylamino)benzamide | 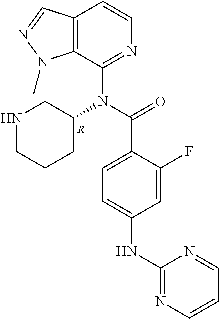 |
| 120 | (R)-2-fluoro-4-(1H-indazol-3-yl)-N-(8- methylisoquinolin-1-yl)-N-(piperidin-3- yl)benzamide | 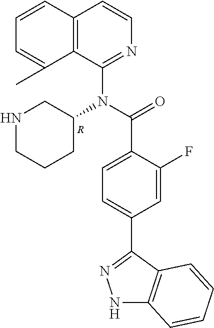 |
| 121 | (R)-4-(3H-[1,2,3]triazolo[4,5-b]pyridin-3- yl)-2-fluoro-N-(3-methylthieno[3,2- c]pyridin-4-yl)-N-(piperidin-3- yl)benzamide | 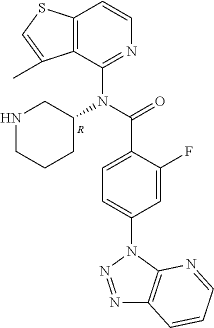 |
| 122 | (R)-4-(3H-[1,2,3]triazolo[4,5-b]pyridin-3- yl)-N-(3-bromothieno[3,2-c]pyridin-4-yl)- 2-fluoro-N-(piperidin-3-yl)benzamide | 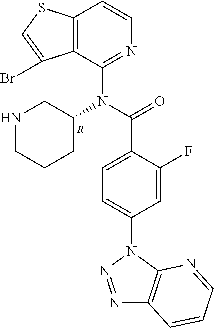 |
| 123 | (R)-2-fluoro-N-(8-methylisoquinolin-1-yl)- N-(piperidin-3-yl)-4-(4,5,6,7-tetrahydro- 1H-indazol-3-yl)benzamide | 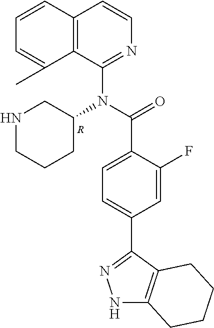 |
| 124 | (R)-4-amino-2-fluoro-N-(8- methylisoquinolin-1-yl)-N-(piperidin-3- yl)benzamide | 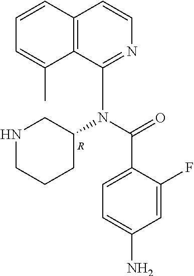 |
| 125 | (R)-2-fluoro-4-(2-methyl-2H-tetrazol-5- yl)-N-(8-methylisoquinolin-1-yl)-N- (piperidin-3-yl)benzamide | 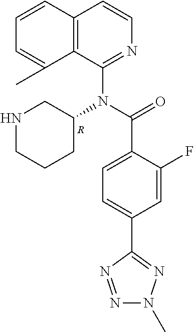 |
| 126 | (R)-2-fluoro-4-(1-methyl-5-phenyl-1H- pyrazol-4-yl)-N-(8-methylisoquinolin-1- yl)-N-(piperidin-3-yl)benzamide | 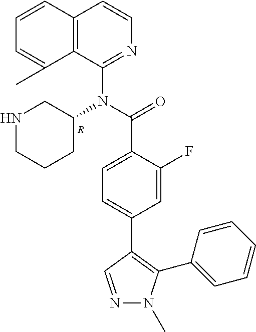 |
| 127 | (R)-2-fluoro-4-(5-methyl-1,3,4-thiadiazol- 2-yl)-N-(8-methylisoquinolin-1-yl)-N- (piperidin-3-yl)benzamide | 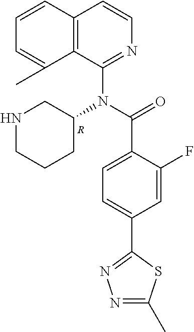 |
| 128 | (R)-N-(8-methylisoquinolin-1-yl)-N- (piperidin-3-yl)-4-(1H-tetrazol-5- yl)benzamide | 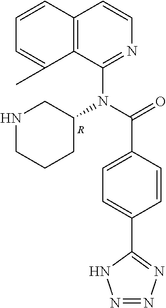 |
| 129 | methyl (R)-1-(4-(3H-[1,2,3]triazolo[4,5- b]pyridin-3-yl)-2-fluoro-N-(piperidin-3- yl)benzamido)isoquinoline-7-carboxylate | 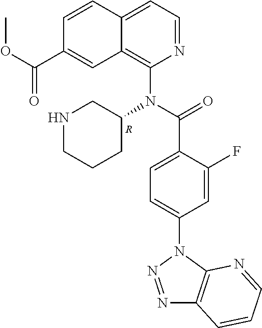 |
| 130 | ethyl (R)-3-(1-(4-(3H-[1,2,3]triazolo[4,5- b]pyridin-3-yl)-2-fluoro-N-(piperidin-3- yl)benzamido)isoquinolin-7-yl)acrylate | 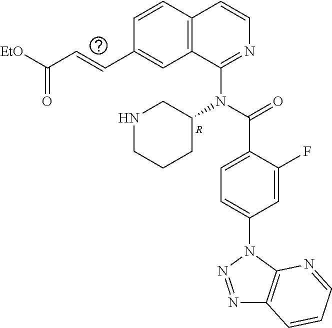 |
| 131 | (R)-2-fluoro-4-((1-methyl-2-oxo-1,2- dihydropyridin-3-yl)amino)-N-(8- methylisoquinolin-1-yl)-N-(piperidin-3- yl)benzamide | 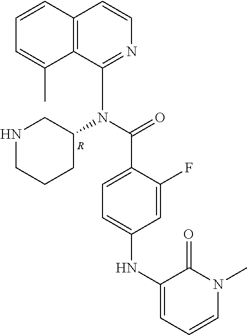 |
| 132 | (R)-2-fluoro-N-(8-methylisoquinolin-1-yl)- 4-((2-oxo-1,2-dihydropyridin-3-yl)amino)- N-(piperidin-3-yl)benzamide | 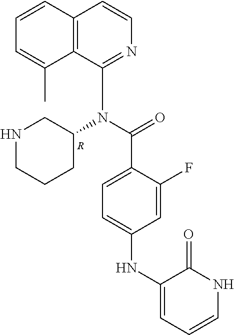 |
| 133 | (R)-2-fluoro-N-(8-methylisoquinolin-1-yl)- N-(piperidin-3-yl)-4-(pyrimidin-2- yloxy)benzamide | 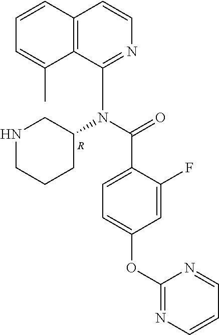 |
| 134 | (R)-4-(3H-[1,2,3]triazolo[4,5-b]pyridin-3- yl)-2-fluoro-N-(7-(3- hydroxypropyl)isoquinolin-1-yl)-N- (piperidin-3-yl)benzamide | 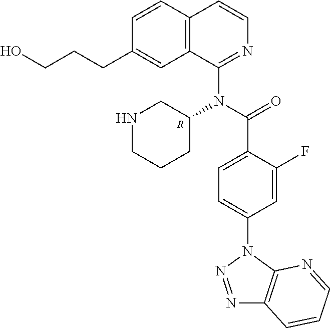 |
| 135 | (R)-4-(3H-[1,2,3]triazolo[4,5-b]pyridin-3- yl)-2-fluoro-N-(7-(3-hydroxyprop-1-en-1- yl)isoquinolin-1-yl)-N-(piperidin-3- yl)benzamide | 468 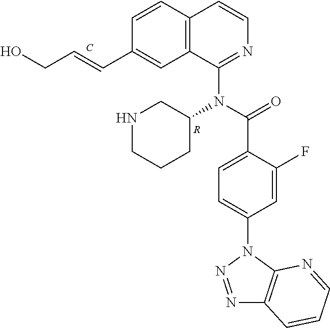 |
| 136 | (R)-N-(3-(1H-pyrazol-3-yl)pyridin-2-yl)-4- (3H-[1,2,3]triazolo[4,5-b]pyridin-3-yl)-2- fluoro-N-(piperidin-3-yl)benzamide | 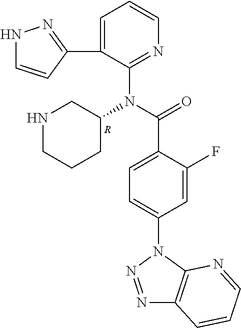 |
| 137 | (R)-4-(3H-[1,2,3]triazolo[4,5-b]pyridin-3- yl)-2-fluoro-N-(3-(4-hydroxybut-1-en-1- yl)-4-methylpyridin-2-yl)-N-(piperidin-3- yeyl)benzamide | 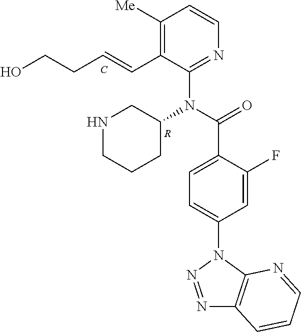 |
| 138 | (R)-4-(5-(1H-pyrazol-3-yl)-3H- [1,2,3]triazolo[4,5-b]pyridin-3-yl)-2- fluoro-N-(8-methylisoquinolin-1-yl)-N- (piperidin-3-yl)benzamide | 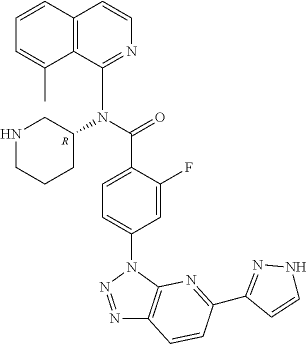 |
| 139 | (R)-3-(3-fluoro-4-((8-methylisoquinolin-1- yl)(piperidin-3-yl)carbamoyl)phenyl)-N- methyl-3H-[1,2,3]triazolo[4,5-b]pyridine- 5-carboxamide | 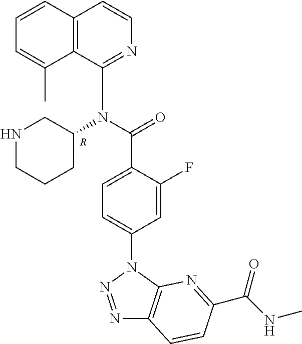 |
| 140 | methyl (R)-3-(3-fluoro-4-((8- methylisoquinolin-1-yl)(piperidin-3- yl)carbamoyl)phenyl)-3H- [1,2,3]triazolo[4,5-b]pyridine-5- carboxylate | 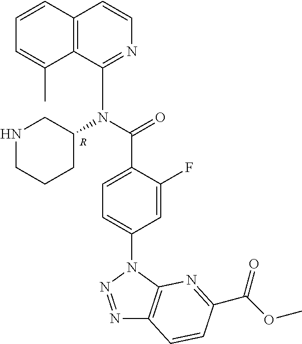 |
| 141 | (R)-N-(8-chloroisoquinolin-1-yl)-2-fluoro- 4-iodo-N-(piperidin-3-yl)benzamide | 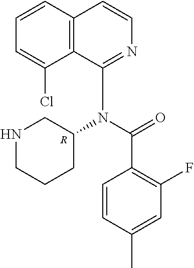 |
| 142 | (R)-4-(5-ethyl-2H-tetrazol-2-yl)-2-fluoro- N-(8-methylisoquinolin-1-yl)-N-(piperidin- 3-yl)benzamide | 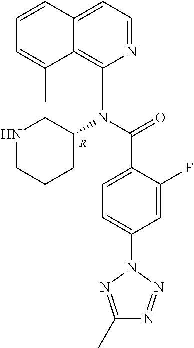 |
| 143 | (R)-4-(3H-[1,2,3]triazolo[4,5-b]pyridin-3- yl)-N-(6-bromoisoquinolin-1-yl)-2-fluoro- N-(piperidin-3-yl)benzamide | 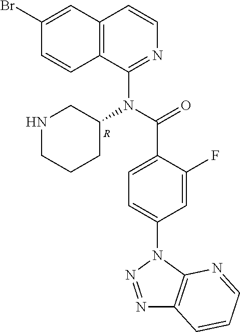 |
| 144 | (R)-4-(3H-[1,2,3]triazolo[4,5-b]pyridin-3- yl)-2-fluoro-N-(6-fluoroisoquinolin-1-yl)- N-(piperidin-3-yl)benzamide | 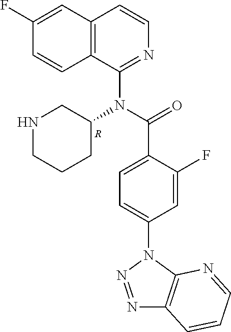 |
| 145 | (R)-4-(3H-[1,2,3]triazolo[4,5-b]pyridin-3- yl)-2-fluoro-N-(6-methoxyisoquinolin-1- yl)-N-(piperidin-3-yl)benzamide | 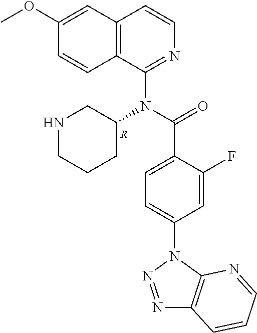 |
| 146 | (R)-4-(3H-[1,2,3]triazolo[4,5-b]pyridin-3- yl)-2-fluoro-N-(6- (hydroxymethyl)isoquinolin-1-yl)-N- (piperidin-3-yl)benzamide | 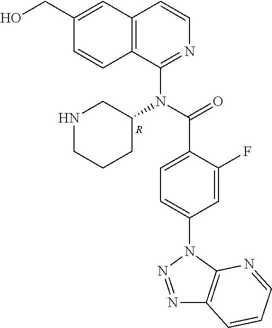 |
| 147 | (R)-4-(3H-[1,2,3]triazolo[4,5-b]pyridin-3- yl)-N-(6-cyanoisoquinolin-1-yl)-2-fluoro- N-(piperidin-3-yl)benzamide | 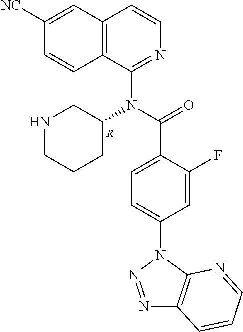 |
| 148 | (R)-4-(3H-[1,2,3]triazolo[4,5-b]pyridin-3- yl)-N-(7-(3-amino-3- oxopropyl)isoquinolin-1-yl)-2-fluoro-N- (piperidin-3-yl)benzamide | 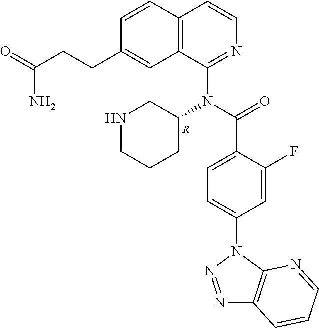 |
| 149 | (R)-4-(3H-[1,2,3]triazolo[4,5-b]pyridin-3- yl)-N-(7-(3-amino-3-oxoprop-1-en-1- yl)isoquinolin-1-yl)-2-fluoro-N-(piperidin- 3-yl)benzamide | 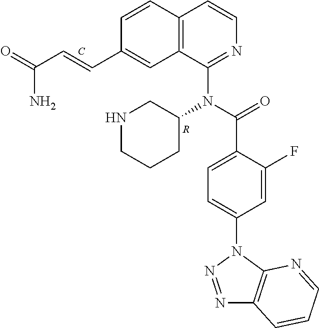 |
| 150 | (R)-4-(3H-[1,2,3]triazolo[4,5-b]pyridin-3- yl)-2-fluoro-N-(piperidin-3-yl)-N-(7- (pyrimidin-5-yl)isoquinolin-1- yl)benzamide | 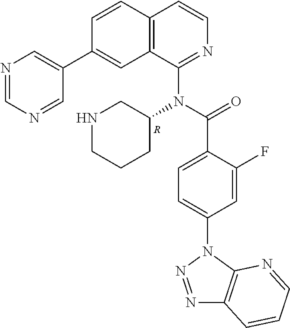 |
| 151 | (R)-4-(3H-[1,2,3]triazolo[4,5-b]pyridin-3- yl)-2-fluoro-N-(piperidin-3-yl)-N-(7- (pyridin-3-yl)isoquinolin-1-yl)benzamide | 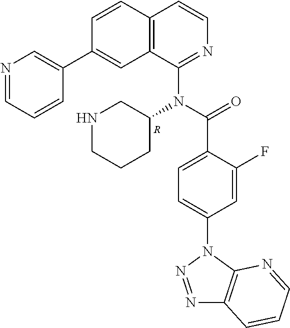 |
| 152 | (R)-N-(7-(1H-pyrazol-5-yl)isoquinolin-1- yl)-4-(3H-[1,2,3]triazolo[4,5-b]pyridin-3- yl)-2-fluoro-N-(piperidin-3-yl)benzamide | 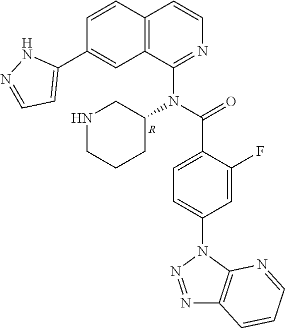 |
| 153 | (R)-(1-(4-(3H-[1,2,3]triazolo[4,5- b]pyridin-3-yl)-2-fluoro-N-(piperidin-3- yl)benzamido)isoquinolin-7-yl)methyl methylcarbamate | 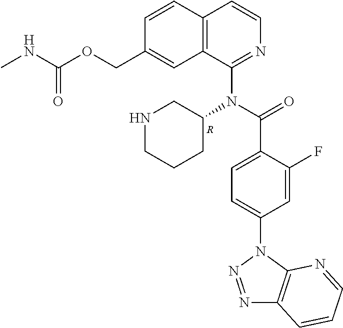 |
| 154 | (R)-4-(3H-[1,2,3]triazolo[4,5-b]pyridin-3- yl)-2-fluoro-N-(7- (hydroxymethyl)isoquinolin-1-yl)-N- (piperidin-3-yl)benzamide | 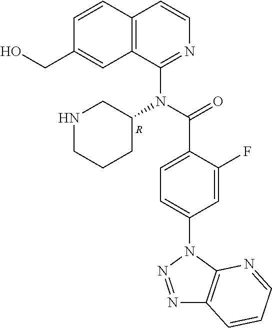 |
| 155 | (R)-1-(4-(3H-[1,2,3]triazolo[4,5-b]pyridin- 3-yl)-2-fluoro-N-(piperidin-3- yl)benzamido)isoquinoline-7-carboxamide | 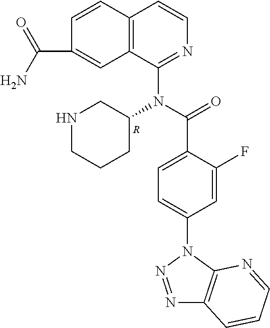 |
| 156 | (R)-4-(3H-[1,2,3]triazolo[4,5-b]pyridin-3- yl)-N-(7-cyanoisoquinolin-1-yl)-2-fluoro- N-(piperidin-3-yl)benzamide | 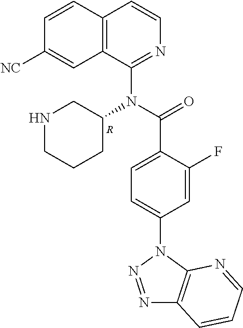 |
| 157 | (R)-4-(3H-[1,2,3]triazolo[4,5-b]pyridin-3- yl)-2-fluoro-N-(piperidin-3-yl)-N-(3- (thieno[3,2-c]pyridin-2-yl)pyridin-2- yl)benzamide | 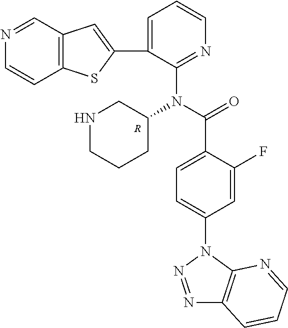 |
| 158 | (R)-4-(3H-[1,2,3]triazolo[4,5-b]pyridin-3- yl)-2-fluoro-N-(2-(3-hydroxyprop-1-en-1- yl)thieno[3,2-c]pyridin-4-yl)-N-(piperidin- 3-yl)benzamide | 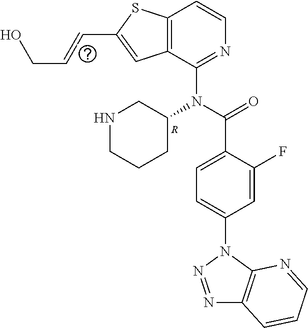 |
| 159 | (R)-4-(3H-[1,2,3]triazolo[4,5-b]pyridin-3- yl)-2-fluoro-N-(piperidin-3-yl)-N-(2- (1,2,3,6-tetrahydropyridin-4-yl)thieno[3,2- c]pyridin-4-yl)benzamide | 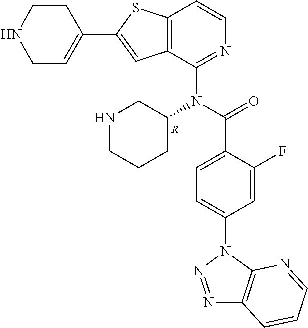 |
| 160 | (R)-N-(2-(1H-pyrazol-4-yl)thieno[3,2- c]pyridin-4-yl)-4-(3H-[1,2,3]triazolo[4,5- b]pyridin-3-yl)-2-fluoro-N-(piperidin-3- yl)benzamide | 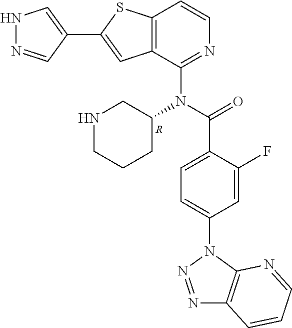 |
| 161 | (R)-4-(3H-[1,2,3]triazolo[4,5-b]pyridin-3- yl)-2-fluoro-N-(piperidin-3-yl)-N-(2- (pyridin-4-yl)thieno[3,2-c]pyridin-4- yl)benzamide | 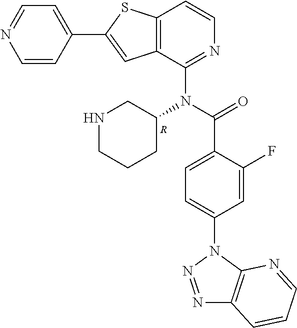 |
| 162 | (R)-4-(3H-[1,2,3]triazolo[4,5-b]pyridin-3- yl)-N-(2-bromothieno[3,2-c]pyridin-4-yl)- 2-fluoro-N-(piperidin-3-yl)benzamide | 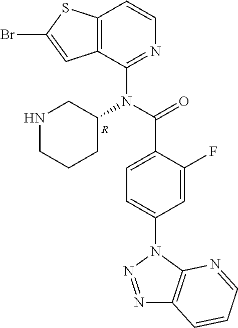 |
| 163 | (R)-4-(3H-[1,2,3]triazolo[4,5-b]pyridin-3- yl)-2-fluoro-N-(piperidin-3-yl)-N-(8-(2- (pyridin-3-yl)vinyl)isoquinolin-1- yl)benzamide | 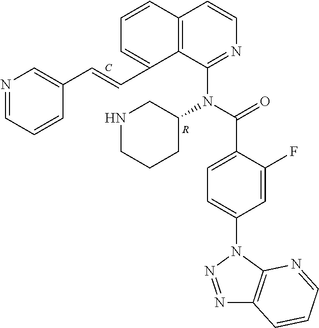 |
| 164 | (R)-4-(3H-[1,2,3]triazolo[4,5-b]pyridin-3- yl)-N-(8-cyanoisoquinolin-1-yl)-2-fluoro- N-(piperidin-3-yl)benzamide | 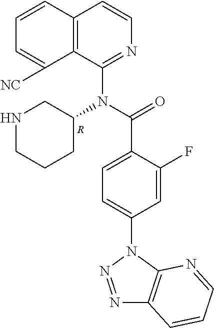 |
| 165 | (R)-4-(3H-[1,2,3]triazolo[4,5-b]pyridin-3- yl)-2-fluoro-N-(8-(3-hydroxyprop-1-en-1- yl)isoquinolin-1-yl)-N-(piperidin-3- yl)benzamide | 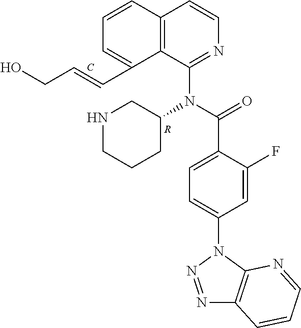 |
| 166 | (R)-4-(3H-[1,2,3]triazolo[4,5-b]pyridin-3- yl)-2-fluoro-N-(8-(4-hydroxybut-1-en-1- yl)isoquinolin-1-yl)-N-(piperidin-3- yl)benzamide | 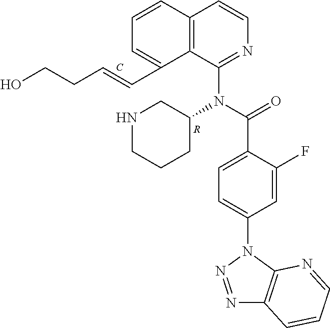 |
| 167 | methyl (R)-2-(4-(3H-[1,2,3]triazolo[4,5- b]pyridin-3-yl)-2-fluoro-N-(piperidin-3- yl)benzamido)nicotinate | 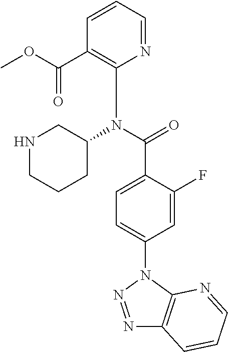 [SMILES: COC(=O)c1cccnc1N(C(=O)c1ccc(-n2nnc3cccnc32)cc1F)[C@@H]1CCCNC1] |
| 168 | (R)-2-fluoro-4-(hydroxymethyl)-N-(8- methylisoquinolin-1-yl)-N-(piperidin-3- yl)benzamide | 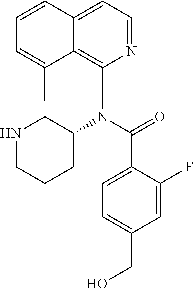 [SMILES: Cc1cccc2ccnc(N(C(=O)c3ccc(CO)cc3F)[C@@H]3CCCNC3)c12] |
| 169 | (R)-4-([1,2,4]triazolo[1,5-a]pyridin-2- ylamino)-2-fluoro-N-(8-methylisoquinolin- 1-yl)-N-(piperidin-3-yl)benzamide | 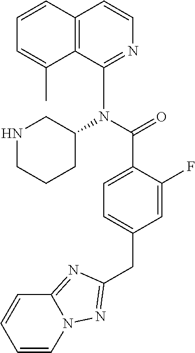 [SMILES: Cc1cccc2ccnc(N(C(=O)c3ccc(Cc4nc5ccccn5n4)cc3F)[C@@H]3CCCNC3)c12] |
| 170 | (R)-3-(3-fluoro-4-((8-methylisoquinolin-1- yl)(piperidin-3-yl)carbamoyl)phenyl)-3H- [1,2,3]triazolo[4,5-b]pyridine-5- carboxamide | 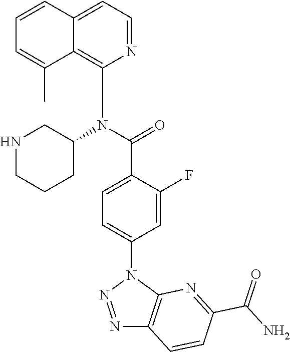 [SMILES: Cc1cccc2ccnc(N(C(=O)c3ccc(-n4nnc5ccc(C(N)=O)nc54)cc3F)[C@@H]3CCCNC3)c12] |
| 171 | (R)-2-fluoro-N-(8-methylisoquinolin-1-yl)- 4-(5-phenyl-3H-[1,2,3]triazolo[4,5- b]pyridin-3-yl)-N-(piperidin-3- yl)benzamide | 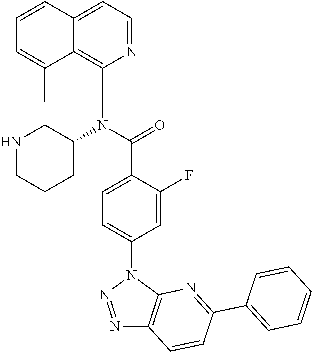 [SMILES: Cc1cccc2ccnc(N(C(=O)c3ccc(-n4nnc5ccc(-c6ccccc6)nc54)cc3F)[C@@H]3CCCNC3)c12] |
| 172 | 2-fluoro-4-((R)-4-methyl-2- oxoimidazolidin-1-yl)-N-(8- methylisoquinolin-1-yl)-N-((R)-piperidin- 3-yl)benzamide | 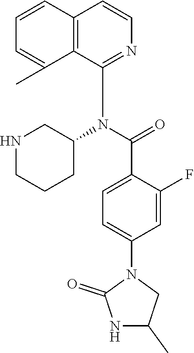 [SMILES: Cc1cccc2ccnc(N(C(=O)c3ccc(N4CC(C)NC4=O)cc3F)[C@@H]3CCCNC3)c12] |
| 173 | 2-fluoro-N-(3-methylpyridin-2-yl)-4- morpholino-N-(piperidin-3-yl)benzamide | 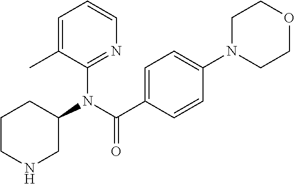 [SMILES: Cc1cccnc1N(C(=O)c1ccc(N2CCOCC2)cc1)[C@@H]1CCCNC1] |
| 174 | (R)-4-(4-methyl-5-oxo-4,5-dihydro-1,3,4- oxadiazol-2-yl)-N-(8-methylisoquinolin-1- yl)-N-(piperidin-3-yl)piperidine-1- carboxamide | 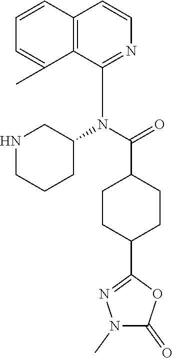 [SMILES: Cc1cccc2ccnc(N(C(=O)C3CCC(c4nn(C)c(=O)o4)CC3)[C@@H]3CCCNC3)c12] |
| 175 | (R)-4-(3H-[1,2,3]triazolo[4,5-b]pyridin-3- yl)-N-(5,5-difluoropiperidin-3-yl)-2-fluoro- N-(8-methylisoquinolin-1-yl)benzamide | 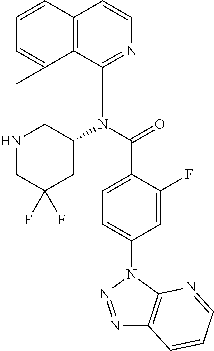 [SMILES: Cc1cccc2ccnc(N(C(=O)c3ccc(-n4nnc5cccnc54)cc3F)[C@H]3CNCC(F)(F)C3)c12] |
| 176 | (R)-N-(3-(1H-pyrazol-4-yl)pyridin-2-yl)-4- (3H-[1,2,3]triazolo[4,5-b]pyridin-3-yl)-2- fluoro-N-(piperidin-3-yl)benzamide | 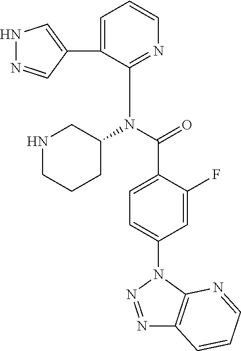 [SMILES: O=C(c1ccc(-n2nnc3cccnc32)cc1F)N(c1ncccc1-c1cn[nH]c1)[C@@H]1CCCNC1] |
| 177 | (R)-4-(3H-[1,2,3]triazolo[4,5-b]pyridin-3- yl)-2-fluoro-N-(piperidin-3-yl)-N-(3- (thiophen-3-yl)pyridin-2-yl)benzamide | 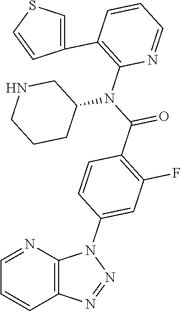 [SMILES: O=C(c1ccc(-n2nnc3cccnc32)cc1F)N(c1ncccc1-c1ccsc1)[C@@H]1CCCNC1] |
| 178 | (R)-4-(3H-[1,2,3]triazolo[4,5-b]pyridin-3- yl)-2-fluoro-N-(piperidin-3-yl)-N-(3- vinylpyridin-2-yl)benzamide | 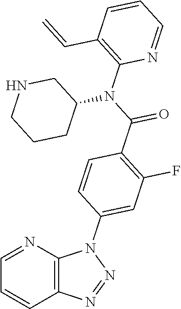 [SMILES: C=Cc1cccnc1N(C(=O)c1ccc(-n2nnc3cccnc32)cc1F)[C@@H]1CCCNC1] |
| 179 | (R)-4-(5-methyl-2H-tetrazol-2-yl)-N-(8- methylisoquinolin-1-yl)-N-(piperidin-3- yl)benzamide | 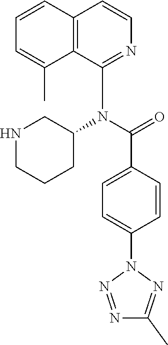 [SMILES: Cc1nnn(-c2ccc(C(=O)N(c3nccc4cccc(C)c34)[C@@H]3CCCNC3)cc2)n1] |
| 180 | (R)-2-fluoro-4-(methyl(4-methylpyrimidin- 2-yl)amino)-N-(8-methylisoquinolin-1-yl)- N-(piperidin-3-yl)benzamide | 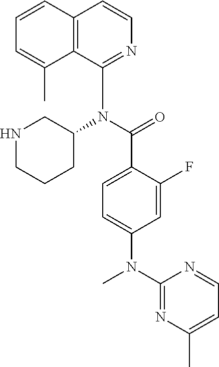 [SMILES: Cc1ccnc(N(C)c2ccc(C(=O)N(c3nccc4cccc(C)c34)[C@@H]3CCCNC3)c(F)c2)n1] |
| 181 | (R)-N-(azepan-3-yl)-N-(8- methylisoquinolin-1-yl)-4-(3- methylisoxazol-5-yl)piperidine-1- carboxamide | 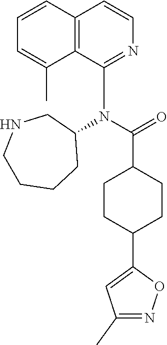 [SMILES: Cc1cc(C2CCC(C(=O)N(c3nccc4cccc(C)c34)[C@@H]3CCCCNC3)CC2)on1] |
| 182 | (R)-N-(8-chloroisoquinolin-1-yl)-2-fluoro- 4-(3-methylpyrazin-2-yl)-N-(piperidin-3- yl)benzamide | 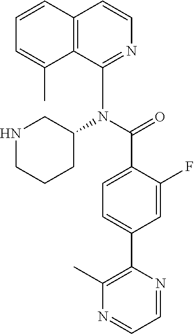 [SMILES: Cc1nccnc1-c1ccc(C(=O)N(c2nccc3cccc(C)c23)[C@@H]2CCCNC2)c(F)c1] |
| 183 | (R)-4-(3-chloropyrazin-2-yl)-2-fluoro-N- (8-methylisoquinolin-1-yl)-N-(piperidin-3- yl)benzamide | 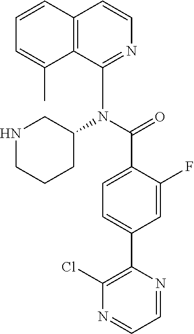 [SMILES: Cc1cccc2ccnc(N(C(=O)c3ccc(-c4nccnc4Cl)cc3F)[C@@H]3CCCNC3)c12] |
| 184 | (R)-N-(8-methylisoquinolin-1-yl)-N- (piperidin-3-yl)-5-(pyrimidin-2- ylamino)picolinamide | 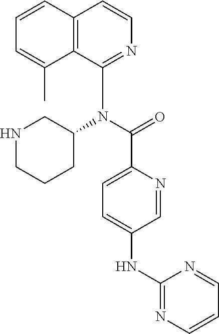 [SMILES: Cc1cccc2ccnc(N(C(=O)c3ccc(Nc4ncccn4)cn3)[C@@H]3CCCNC3)c12] |
| 185 | (R)-N-(azepan-3-yl)-2-fluoro-N-(8- methylisoquinolin-1-yl)-4-(pyrimidin-2- ylamino)benzamide | 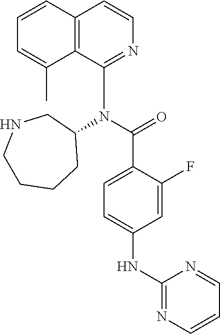 [SMILES: Cc1cccc2ccnc(N(C(=O)c3ccc(Nc4ncccn4)cc3F)[C@@H]3CCCCNC3)c12] |
| 186 | (R)-2-fluoro-N-(8-methylisoquinolin-1-yl)- 4-((5-methylpyrimidin-2-yl)amino)-N- (piperidin-3-yl)benzamide | 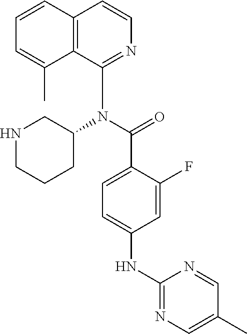 [SMILES: Cc1cnc(Nc2ccc(C(=O)N(c3nccc4cccc(C)c34)[C@@H]3CCCNC3)c(F)c2)nc1] |
| 187 | (R)-2-fluoro-4-(5-methoxy-3H- [1,2,3]triazolo[4,5-b]pyridin-3-yl)-N-(8- methylisoquinolin-1-yl)-N-(piperidin-3- yl)benzamide | 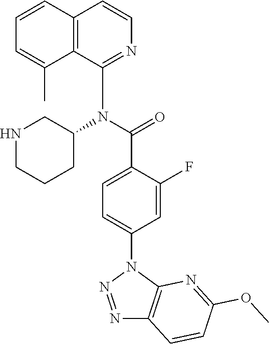 [SMILES: COc1ccc2nnn(-c3ccc(C(=O)N(c4nccc5cccc(C)c45)[C@@H]4CCCNC4)c(F)c3)c2n1] |
| 188 | (S)-8a-methyl-N-(8-methylisoquinolin-1- yl)-3-oxo-N-((R)-piperidin-3- yl)tetrahydro-3H-oxazolo[3,4-a]pyrazine- 7(1H)-carboxamide | 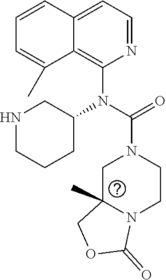 [SMILES: Cc1cccc2ccnc(N(C(=O)N3CCN4C(=O)OC[C@]4(C)C3)[C@@H]3CCCNC3)c12] |
| 189 | (R)-N-(8-methylisoquinolin-1-yl)-4- phenoxy-N-(piperidin-3-yl)piperidine-1- carboxamide | 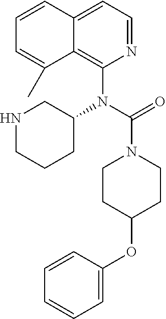 [SMILES: Cc1cccc2ccnc(N(C(=O)N3CCC(Oc4ccccc4)CC3)[C@@H]3CCCNC3)c12] |
| 190 | (R)-N-(isoquinolin-3-yl)-4-(isoxazol-5-yl)- 4-methyl-N-(piperidin-3-yl)piperidine-1- carboxamide | 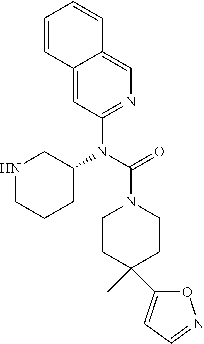 [SMILES: CC1(c2ccno2)CCN(C(=O)N(c2cc3ccccc3cn2)[C@@H]2CCCNC2)CC1] |
| 191 | (R)-4-(5-cyano-3H-[1,2,3]triazolo[4,5- b]pyridin-3-yl)-2-fluoro-N-(8- methylisoquinolin-1-yl)-N-(piperidin-3- yl)benzamide | 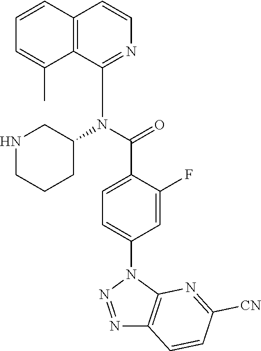 [SMILES: Cc1cccc2ccnc(N(C(=O)c3ccc(-n4nnc5ccc(C#N)nc54)cc3F)[C@@H]3CCCNC3)c12] |
| 192 | (R)-4-(5-chloro-3H-[1,2,3]triazolo[4,5- b]pyridin-3-yl)-2-fluoro-N-(8- methylisoquinolin-1-yl)-N-(piperidin-3- yl)benzamide | 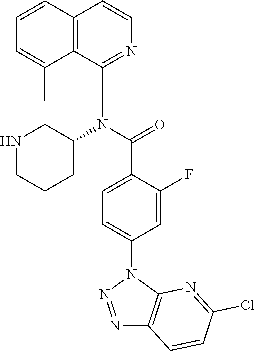 [SMILES: Cc1cccc2ccnc(N(C(=O)c3ccc(-n4nnc5ccc(Cl)nc54)cc3F)[C@@H]3CCCNC3)c12] |
| 193 | (R)-2-fluoro-4-(1-methyl-1H-1,2,3-triazol- 4-yl)-N-(8-methylisoquinolin-1-yl)-N- (piperidin-3-yl)benzamide | 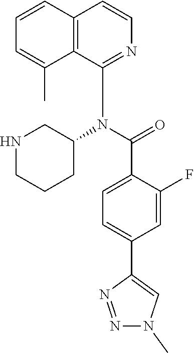 [SMILES: Cc1cccc2ccnc(N(C(=O)c3ccc(-c4cn(C)nn4)cc3F)[C@@H]3CCCNC3)c12] |
| 194 | (R)-2-fluoro-N-(8-methylisoquinolin-1-yl)- N-(piperidin-3-yl)-4-(2H-1,2,3-triazol-2- yl)benzamide | 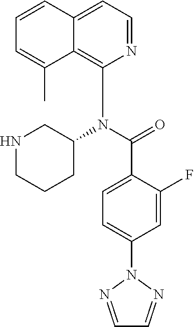 [SMILES: Cc1cccc2ccnc(N(C(=O)c3ccc(-n4nccn4)cc3F)[C@@H]3CCCNC3)c12] |
| 195 | (R)-2-fluoro-4-(5-methyl-2H-tetrazol-2- yl)-N-(8-methylisoquinolin-1-yl)-N- (piperidin-3-yl)benzamide | 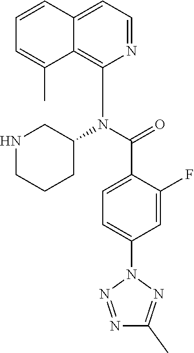 [SMILES: Cc1nnn(-c2ccc(C(=O)N(c3nccc4cccc(C)c34)[C@@H]3CCCNC3)c(F)c2)n1] |
| 196 | (R)-4-(benzo[c]isoxazol-3-yl)-2-fluoro-N- (8-methylisoquinolin-1-yl)-N-(piperidin-3- yl)benzamide | 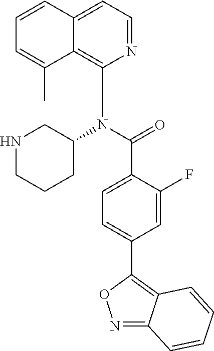 [SMILES: Cc1cccc2ccnc(N(C(=O)c3ccc(-c4onc5ccccc45)cc3F)[C@@H]3CCCNC3)c12] |
| 197 | (R)-N-(8-methylisoquinolin-1-yl)-4-(3- methylisoxazol-5-yl)-N-(piperidin-3-yl)- 3,6-dihydropyridine-1(2H)-carboxamide | 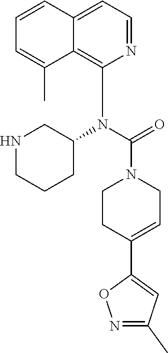 [SMILES: Cc1cc(C2=CCN(C(=O)N(c3nccc4cccc(C)c34)[C@@H]3CCCNC3)CC2)on1] |
| 198 | (R)-4-(6-cyanopyrazin-2-yl)-2-fluoro-N- (8-methylisoquinolin-1-yl)-N-(piperidin-3- yl)benzamide | 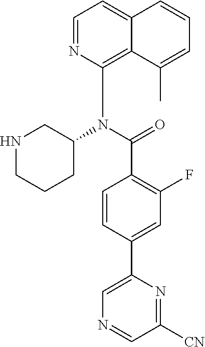 [SMILES: Cc1cccc2ccnc(N(C(=O)c3ccc(-c4cncc(C#N)n4)cc3F)[C@@H]3CCCNC3)c12] |
| 199 | (R)-N-(8-methylisoquinolin-1-yl)-4-(2- methylpyridin-4-yl)-N-(piperidin-3- yl)piperidine-1-carboxamide | 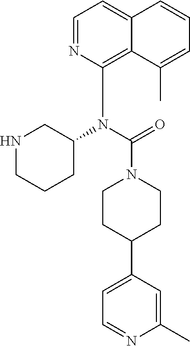 [SMILES: Cc1cc(C2CCN(C(=O)N(c3nccc4cccc(C)c34)[C@@H]3CCCNC3)CC2)ccn1] |
| 200 | (R)-5-(3H-[1,2,3]triazolo[4,5-b]pyridin-3- yl)-N-(8-chloroisoquinolin-1-yl)-3-fluoro- N-(piperidin-3-yl)picolinamide | 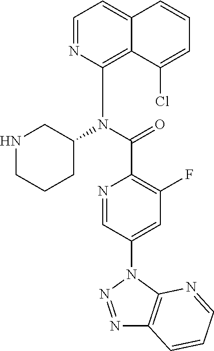 [SMILES: O=C(c1ncc(-n2nnc3cccnc32)cc1F)N(c1nccc2cccc(Cl)c12)[C@@H]1CCCNC1] |
| 201 | (R)-5-(3H-[1,2,3]triazolo[4,5-b]pyridin-3- yl)-3-fluoro-N-(8-methylisoquinolin-1-yl)- N-(piperidin-3-yl)picolinamide | 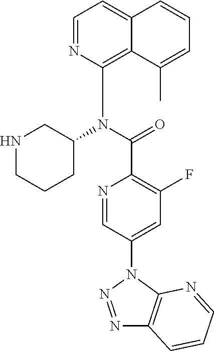 [SMILES: Cc1cccc2ccnc(N(C(=O)c3ncc(-n4nnc5cccnc54)cc3F)[C@@H]3CCCNC3)c12] |
| 202 | (R)-2-fluoro-N-(8-methylisoquinolin-1-yl)- 4-(6-methylpyrazin-2-yl)-N-(piperidin-3- yl)benzamide | 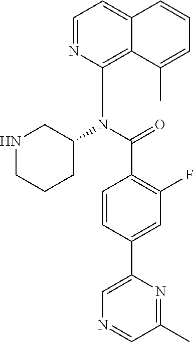 [SMILES: Cc1cncc(-c2ccc(C(=O)N(c3nccc4cccc(C)c34)[C@@H]3CCCNC3)c(F)c2)n1] |
| 203 | (R)-2-fluoro-N-(8-methylisoquinolin-1-yl)- 4-(3-methylpyrazin-2-yl)-N-(piperidin-3- yl)benzamide | 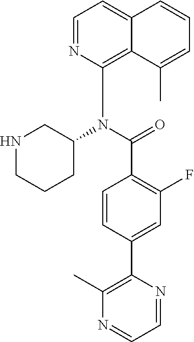 [SMILES: Cc1nccnc1-c1ccc(C(=O)N(c2nccc3cccc(C)c23)[C@@H]2CCCNC2)c(F)c1] |
| 204 | (R)-2-fluoro-N-(8-methylisoquinolin-1-yl)- N-(piperidin-3-yl)-4-(pyrazin-2- yl)benzamide | 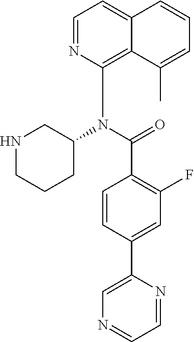 [SMILES: Cc1cccc2ccnc(N(C(=O)c3ccc(-c4cnccn4)cc3F)[C@@H]3CCCNC3)c12] |
| 205 | (R)-2-fluoro-N-(8-methylisoquinolin-1-yl)- N-(piperidin-3-yl)-4-(5-(trifluoromethyl)- 3H-[1,2,3]triazolo[4,5-b]pyridin-3- yl)benzamide | 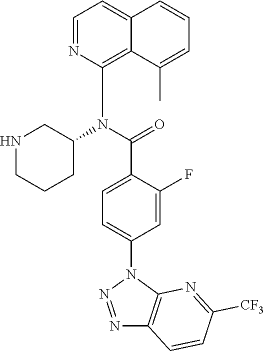 [SMILES: Cc1cccc2ccnc(N(C(=O)c3ccc(-n4nnc5ccc(C(F)(F)F)nc54)cc3F)[C@@H]3CCCNC3)c12] |
| 206 | (S)-4-(4-(4-(dimethylamino)but-2- enamido)phenyl)-N-(1-methyl-1H- pyrrolo[2,3-c]pyridin-7-yl)-N-(piperidin-3- yl)piperidine-1-carboxamide | 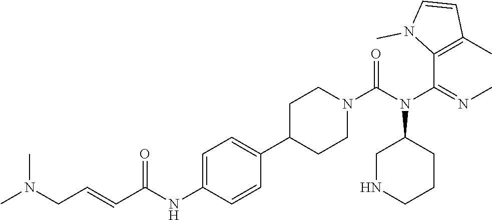 |
| 207 | (R)-4-(3-(4-(dimethylamino)but-2- enamido)phenyl)-N-(8-methylisoquinolin- 1-yl)-N-(piperidin-3-yl)piperidine-1- carboxamide | 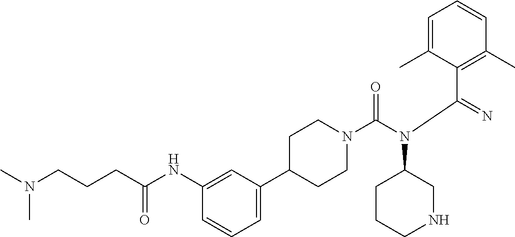 |
| 208 | (R)-N-(8-methylisoquinolin-1-yl)-N- (piperidin-3-yl)-4-(pyrimidin-2- ylamino)piperidine-1-carboxamide | 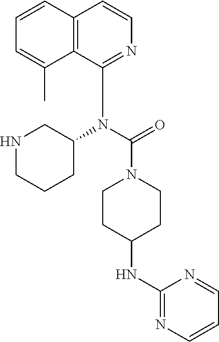 [SMILES: Cc1cccc2ccnc(N(C(=O)N3CCC(Nc4ncccn4)CC3)[C@@H]3CCCNC3)c12] |
| 209 | (R)-2-fluoro-N-(1-methyl-1H-pyrrolo[2,3- c]pyridin-7-yl)-4-(5-methyl-3H- [1,2,3]triazolo[4,5-b]pyridin-3-yl)-N- (piperidin-3-yl)benzamide | 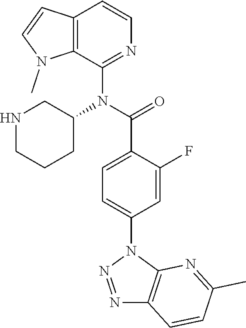 [SMILES: Cc1ccc2nnn(-c3ccc(C(=O)N(c4nccc5ccn(C)c45)[C@@H]4CCCNC4)c(F)c3)c2n1] |
| 210 | (R)-2-fluoro-N-(8-methylisoquinolin-1-yl)- N-(piperidin-3-yl)-4-(pyrimidin-2- ylamino)benzamide | 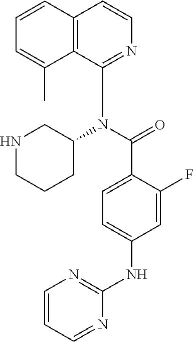 [SMILES: Cc1cccc2ccnc(N(C(=O)c3ccc(Nc4ncccn4)cc3F)[C@@H]3CCCNC3)c12] |
| 211 | (R)-2-fluoro-4-(1-methyl-1H-pyrazol-4- yl)-N-(1-methyl-1H-pyrrolo[2,3-c]pyridin- 7-yl)-N-(piperidin-3-yl)benzamide | 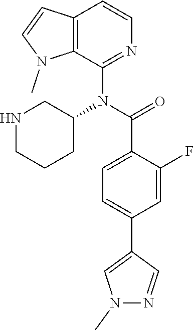 [SMILES: Cn1cc(-c2ccc(C(=O)N(c3nccc4ccn(C)c34)[C@@H]3CCCNC3)c(F)c2)cn1] |
| 212 | (R)-2-fluoro-N-(1-methyl-1H-pyrrolo[2,3- c]pyridin-7-yl)-4-(3-methylisoxazol-5-yl)- N-(piperidin-3-yl)benzamide | 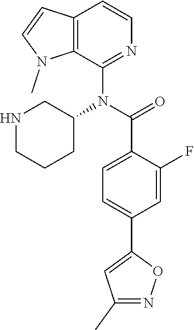 [SMILES: Cc1cc(-c2ccc(C(=O)N(c3nccc4ccn(C)c34)[C@@H]3CCCNC3)c(F)c2)on1] |
| 213 | (R)-2-(4-(3H-[1,2,3]triazolo[4,5-b]pyridin- 3-yl)-2-fluoro-N-(piperidin-3- yl)benzamido)-N-methylnicotinamide | 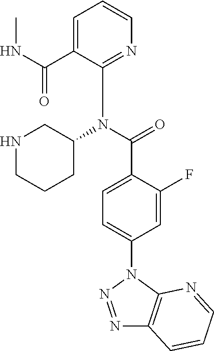 [SMILES: CNC(=O)c1cccnc1N(C(=O)c1ccc(-n2nnc3cccnc32)cc1F)[C@@H]1CCCNC1] |
| 214 | (R)-2-fluoro-4-(1-methyl-1H-pyrazol-4- yl)-N-(8-methylisoquinolin-1-yl)-N- (piperidin-3-yl)benzamide | 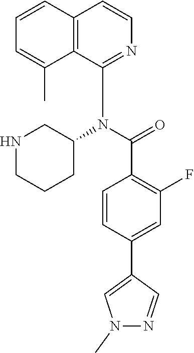 [SMILES: Cc1cccc2ccnc(N(C(=O)c3ccc(-c4cnn(C)c4)cc3F)[C@@H]3CCCNC3)c12] |
| 215 | (R)-2-fluoro-N-(8-methylisoquinolin-1-yl)- N-(piperidin-3-yl)-4-(pyrazolo[1,5- alpyrimidin-3-yl)benzamide | 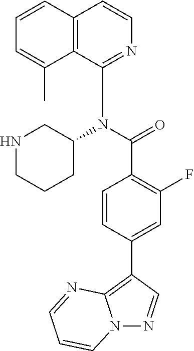 [SMILES: Cc1cccc2ccnc(N(C(=O)c3ccc(-c4cnn5cccnc45)cc3F)[C@@H]3CCCNC3)c12] |
| 216 | (R)-4-(5-ethyl-3H-[1,2,3]triazolo[4,5- b]pyridin-3-yl)-2-fluoro-N-(8- methylisoquinolin-1-yl)-N-(piperidin-3- yl)benzamide | 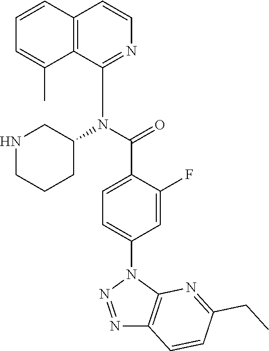 [SMILES: CCc1ccc2nnn(-c3ccc(C(=O)N(c4nccc5cccc(C)c45)[C@@H]4CCCNC4)c(F)c3)c2n1] |
| 217 | (R)-2-fluoro-4-(5-methyl-3H- [1,2,3]triazolo[4,5-b]pyridin-3-yl)-N-(8- methylisoquinolin-1-yl)-N-(piperidin-3- yl)benzamide | 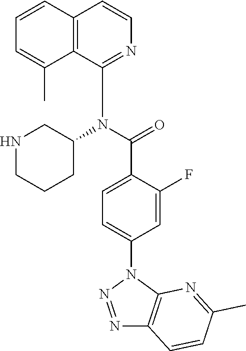 [SMILES: Cc1ccc2nnn(-c3ccc(C(=O)N(c4nccc5cccc(C)c45)[C@@H]4CCCNC4)c(F)c3)c2n1] |
| 218 | (R)-4-(3H-[1,2,3]triazolo[4,5-b]pyridin-3- yl)-2,6-difluoro-N-(8-methylisoquinolin-1- yl)-N-(piperidin-3-yl)benzamide | 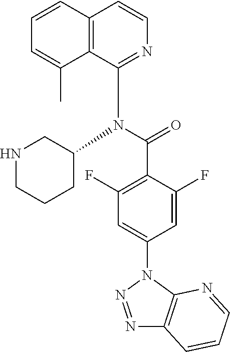 [SMILES: Cc1cccc2ccnc(N(C(=O)c3c(F)cc(-n4nnc5cccnc54)cc3F)[C@@H]3CCCNC3)c12] |
| 219 | (R)-N-(8-methylisoquinolin-1-yl)-4- morpholino-N-(piperidin-3-yl)benzamide | 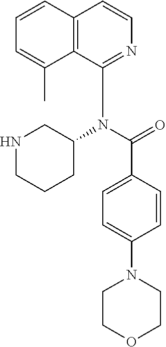 [SMILES: Cc1cccc2ccnc(N(C(=O)c3ccc(N4CCOCC4)cc3)[C@@H]3CCCNC3)c12] |
| 220 | (R)-N-(8-methylisoquinolin-1-yl)-N- (piperidin-3-yl)quinoline-7-carboxamide | 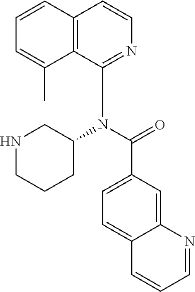 [SMILES: Cc1cccc2ccnc(N(C(=O)c3ccc4cccnc4c3)[C@@H]3CCCNC3)c12] |
| 221 | (R)-4-(3H-[1,2,3]triazolo[4,5-b]pyridin-3- yl)-2-fluoro-N-(piperidin-3-yl)-N-(8- (trifluoromethyl)isoquinolin-1- yl)benzamide | 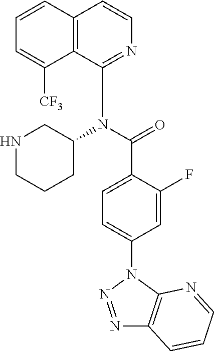 [SMILES: O=C(c1ccc(-n2nnc3cccnc32)cc1F)N(c1nccc2cccc(C(F)(F)F)c12)[C@@H]1CCCNC1] |
| 222 | (R)-4-(3H-[1,2,3]triazolo[4,5-b]pyridin-3- yl)-2-fluoro-N-(8-methoxyisoquinolin-1- yl)-N-(piperidin-3-yl)benzamide | 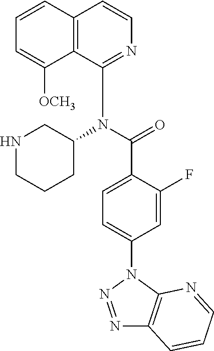 [SMILES: COc1cccc2ccnc(N(C(=O)c3ccc(-n4nnc5cccnc54)cc3F)[C@@H]3CCCNC3)c12] |
| 223 | (R)-4-(1H-benzo[d][1,2,3]triazol-1-yl)-N- (8-methylisoquinolin-1-yl)-N-(piperidin-3- yl)benzamide | 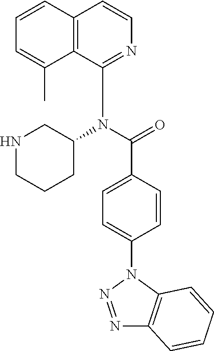 [SMILES: Cc1cccc2ccnc(N(C(=O)c3ccc(-n4nnc5ccccc54)cc3)[C@@H]3CCCNC3)c12] |
| 224 | 4-(3H-[1,2,3]triazolo[4,5-b]pyridin-3-yl)- N-(8-methylisoquinolin-1-yl)-N-((3R,4S)- 4-methylpiperidin-3-yl)benzamide | 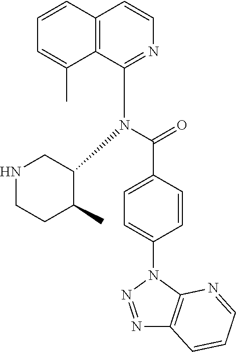 [SMILES: Cc1cccc2ccnc(N(C(=O)c3ccc(-n4nnc5cccnc54)cc3)[C@H]3CNCC[C@@H]3C)c12] |
| 225 | 4-(3H-[1,2,3]triazolo[4,5-b]pyridin-3-yl)- N-(8-methylisoquinolin-1-yl)-N-((3R,5S)- 5-methylpiperidin-3-yl)benzamide | 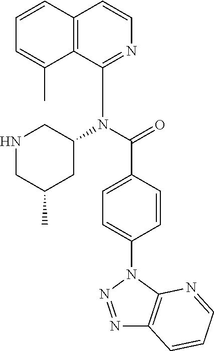 [SMILES: Cc1cccc2ccnc(N(C(=O)c3ccc(-n4nnc5cccnc54)cc3)[C@H]3CNC[C@@H](C)C3)c12] |
| 226 | 4-(3H-[1,2,3]triazolo[4,5-b]pyridin-3-yl)- N-(8-methylisoquinolin-1-yl)-N-((3R,6R)- 6-methylpiperidin-3-yl)benzamide | 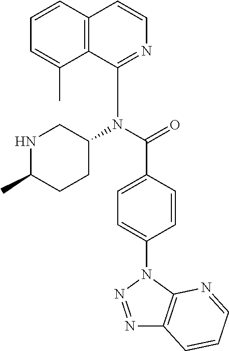 [SMILES: Cc1cccc2ccnc(N(C(=O)c3ccc(-n4nnc5cccnc54)cc3)[C@@H]3CC[C@@H](C)NC3)c12] |
| 227 | (S)-4-(3H-[1,2,3]triazolo[4,5-b]pyridin-3- yl)-N-(azepan-3-yl)-N-(8- methylisoquinolin-1-yl)benzamide | 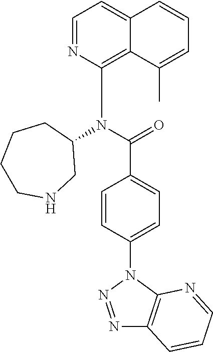 [SMILES: Cc1cccc2ccnc(N(C(=O)c3ccc(-n4nnc5cccnc54)cc3)[C@H]3CCCCNC3)c12] |
| 228 | (S)-4-(3H-[1,2,3]triazolo[4,5-b]pyridin-3- yl)-N-(azepan-4-yl)-N-(8- methylisoquinolin-1-yl)benzamide | 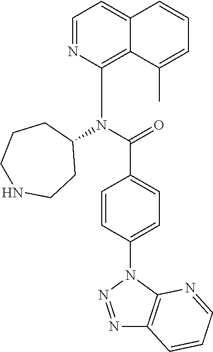 [SMILES: Cc1cccc2ccnc(N(C(=O)c3ccc(-n4nnc5cccnc54)cc3)[C@H]3CCCNCC3)c12] |
| 229 | (R)-4-(3H-[1,2,3]triazolo[4,5-b]pyridin-3- yl)-N-(azepan-4-yl)-N-(8- methylisoquinolin-1-yl)benzamide | 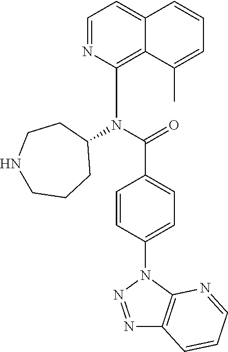 [SMILES: Cc1cccc2ccnc(N(C(=O)c3ccc(-n4nnc5cccnc54)cc3)[C@@H]3CCCNCC3)c12] |
| 230 | (R)-4-(3H-[1,2,3]triazolo[4,5-b]pyridin-3- yl)-N-(azepan-3-yl)-N-(8- methylisoquinolin-1-yl)benzamide | 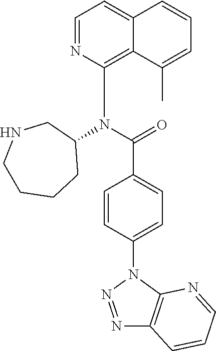 [SMILES: Cc1cccc2ccnc(N(C(=O)c3ccc(-n4nnc5cccnc54)cc3)[C@@H]3CCCCNC3)c12] |
| 231 | (S)-4-(3H-[1,2,3]triazolo[4,5-b]pyridin-3- yl)-N-(8-methylisoquinolin-1-yl)-N- (pyrrolidin-3-yl)benzamide | 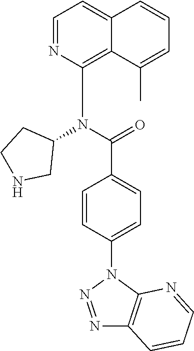 [SMILES: Cc1cccc2ccnc(N(C(=O)c3ccc(-n4nnc5cccnc54)cc3)[C@H]3CCNC3)c12] |
| 232 | (R)-4-(3H-[1,2,3]triazolo[4,5-b]pyridin-3- yl)-N-(8-methylisoquinolin-1-yl)-N- (pyrrolidin-3-yl)benzamide | 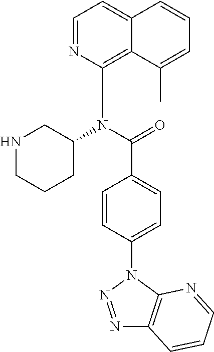 [SMILES: Cc1cccc2ccnc(N(C(=O)c3ccc(-n4nnc5cccnc54)cc3)[C@@H]3CCCNC3)c12] |
| 233 | (R)-N-(8-methylisoquinolin-1-yl)-N- (piperidin-3-yl)-4-(pyridin-3-yl)benzamide | 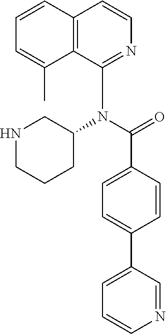 [SMILES: Cc1cccc2ccnc(N(C(=O)c3ccc(-c4cccnc4)cc3)[C@@H]3CCCNC3)c12] |
| 234 | (R)-N-(8-methylisoquinolin-1-yl)-N- (piperidin-3-yl)-4-(2H-tetrazol-2- yl)benzamide | 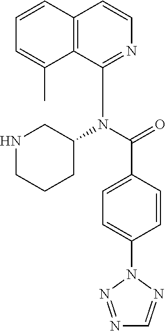 [SMILES: Cc1cccc2ccnc(N(C(=O)c3ccc(-n4ncnn4)cc3)[C@@H]3CCCNC3)c12] |
| 235 | (R)-N-(8-methylisoquinolin-1-yl)-N- (piperidin-3-yl)-4-(2H-1,2,3-triazol-2- yl)benzamide | 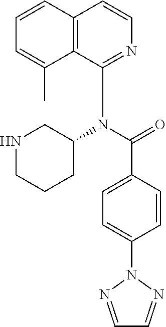 [SMILES: Cc1cccc2ccnc(N(C(=O)c3ccc(-n4nccn4)cc3)[C@@H]3CCCNC3)c12] |
| 236 | (R)-4-(4-methyl-1H-1,2,3-triazol-1-yl)-N- (8-methylisoquinolin-1-yl)-N-(piperidin-3- yl)benzamide | 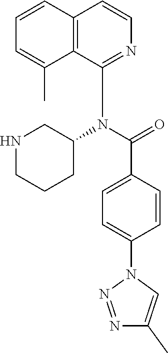 [SMILES: Cc1cn(-c2ccc(C(=O)N(c3nccc4cccc(C)c34)[C@@H]3CCCNC3)cc2)nn1] |
| 237 | (R)-N-(8-methylisoquinolin-1-yl)-N- (piperidin-3-yl)-4-(pyridazin-3- yl)benzamide | 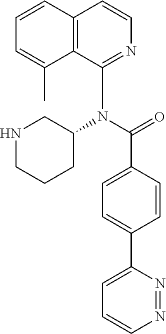 [SMILES: Cc1cccc2ccnc(N(C(=O)c3ccc(-c4cccnn4)cc3)[C@@H]3CCCNC3)c12] |
| 238 | (R)-N-(8-methylisoquinolin-1-yl)-N- (piperidin-3-yl)-4-(pyrazin-2-yl)benzamide | 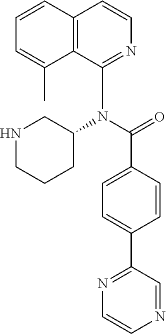 [SMILES: Cc1cccc2ccnc(N(C(=O)c3ccc(-c4cnccn4)cc3)[C@@H]3CCCNC3)c12] |
| 239 | (R)-4-(1-methyl-1H-pyrazol-4-yl)-N-(8- methylisoquinolin-1-yl)-N-(piperidin-3- yl)benzamide | 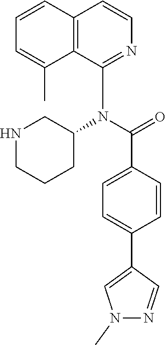 [SMILES: Cc1cccc2ccnc(N(C(=O)c3ccc(-c4cnn(C)c4)cc3)[C@@H]3CCCNC3)c12] |
| 240 | (R)-N-(8-methylisoquinolin-1-yl)-N- (piperidin-3-yl)-3,4-dihydroisoquinoline- 2(1H)-carboxamide | 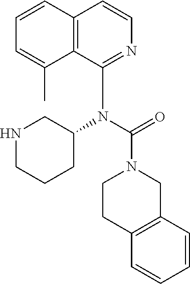 [SMILES: Cc1cccc2ccnc(N(C(=O)N3CCc4ccccc4C3)[C@@H]3CCCNC3)c12] |
| 241 | (R)-4-(3H-[1,2,3]triazolo[4,5-b]pyridin-3- yl)-N-(8-methylisoquinolin-1-yl)-N- (piperidin-3-yl)benzamide (Example | 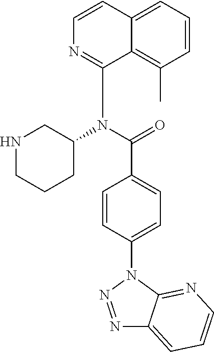 [SMILES: Cc1cccc2ccnc(N(C(=O)c3ccc(-n4nnc5cccnc54)cc3)[C@@H]3CCCNC3)c12] |
| 242 | (R)-4-(3H-[1,2,3]triazolo[4,5-b]pyridin-3- yl)-2-fluoro-N-(8-methylisoquinolin-1-yl)- N-(piperidin-3-yl)benzamide | 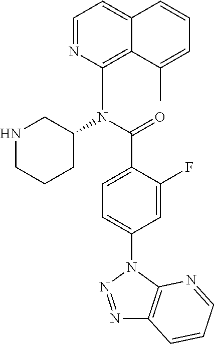 [SMILES: Cc1cccc2ccnc(N(C(=O)c3ccc(-n4nnc5cccnc54)cc3F)[C@@H]3CCCNC3)c12] |
| 243 | (R)-N-(8-methylisoquinolin-1-yl)-4-(3- methylisoxazol-5-yl)-N-(piperidin-3- yl)piperidine-1-carboxamide | 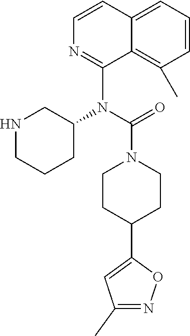 [SMILES: Cc1cc(C2CCN(C(=O)N(c3nccc4cccc(C)c34)[C@@H]3CCCNC3)CC2)on1] |
| 244 | (R)-5-(3H-[1,2,3]triazolo[4,5-b]pyridin-3- yl)-N-(8-methylisoquinolin-1-yl)-N- (piperidin-3-yl)picolinamide | 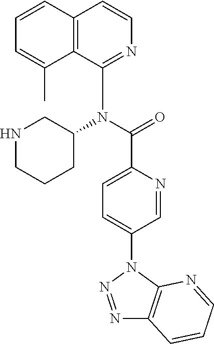 [SMILES: Cc1cccc2ccnc(N(C(=O)c3ccc(-n4nnc5cccnc54)cn3)[C@@H]3CCCNC3)c12] |
| 245 | (R)-6-(3H-[1,2,3]triazolo[4,5-b]pyridin-3- yl)-N-(8-methylisoquinolin-1-yl)-N- (piperidin-3-yl)nicotinamide | 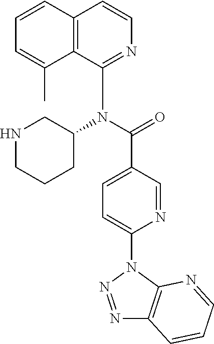 [SMILES: Cc1cccc2ccnc(N(C(=O)c3ccc(-n4nnc5cccnc54)nc3)[C@@H]3CCCNC3)c12] |
| 246 | (R)-4-(3H-[1,2,3]triazolo[4,5-b]pyridin-3- yl)-N-(8-chloroisoquinolin-1-yl)-2-fluoro- N-(piperidin-3-yl)benzamide | 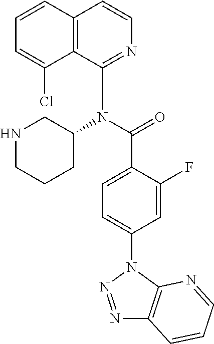 [SMILES: O=C(c1ccc(-n2nnc3cccnc32)cc1F)N(c1nccc2cccc(Cl)c12)[C@@H]1CCCNC1] |
| 247 | (R)-2-(4-(3H-[1,2,3]triazolo[4,5-b]pyridin- 3-yl)-2-fluoro-N-(piperidin-3- yl)benzamido)-N,N-dimethylnicotinamide | 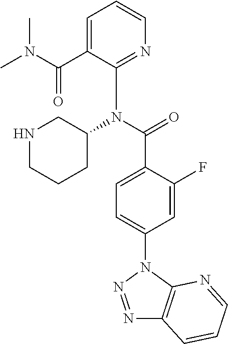 [SMILES: CN(C)C(=O)c1cccnc1N(C(=O)c1ccc(-n2nnc3cccnc32)cc1F)[C@@H]1CCCNC1] |
| 248 | (R)-4-(3H-[1,2,3]triazolo[4,5-b]pyridin-3- yl)-2-fluoro-N-(3-methyl-3H-imidazo[4,5- c]pyridin-4-yl)-N-(piperidin-3- yl)benzamide | 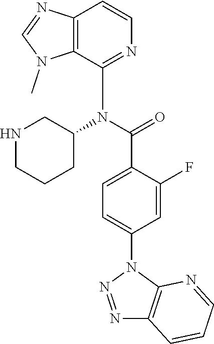 [SMILES: Cn1cnc2ccnc(N(C(=O)c3ccc(-n4nnc5cccnc54)cc3F)[C@@H]3CCCNC3)c21] |
| 249 | (R)-4-(3H-[1,2,3]triazolo[4,5-b]pyridin-3- yl)-2-fluoro-N-(1,6-naphthyridin-5-yl)-N- (piperidin-3-yl)benzamide | 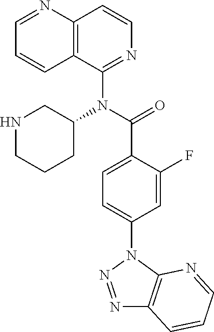 [SMILES: O=C(c1ccc(-n2nnc3cccnc32)cc1F)N(c1nccc2ncccc12)[C@@H]1CCCNC1] |
| 250 | (R)-4-(3-(difluoromethyl)isoxazol-5-yl)-N- (8-methylisoquinolin-1-yl)-N-(piperidin-3- yl)piperidine-1-carboxamide | 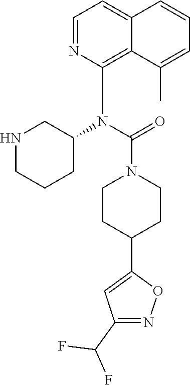 [SMILES: Cc1cccc2ccnc(N(C(=O)N3CCC(c4cc(C(F)F)no4)CC3)[C@@H]3CCCNC3)c12] |
| 251 | (R)-2-fluoro-4-(4-methyl-1H-1,2,3-triazol- 1-yl)-N-(8-methylisoquinolin-1-yl)-N- (piperidin-3-yl)benzamide | 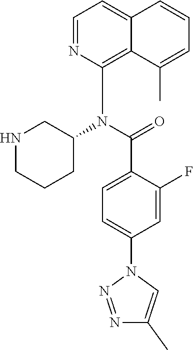 [SMILES: Cc1cn(-c2ccc(C(=O)N(c3nccc4cccc(C)c34)[C@@H]3CCCNC3)c(F)c2)nn1] |
| 252 | (R)-4-(3H-[1,2,3]triazolo[4,5-b]pyridin-3- yl)-N-(4,8-dimethylisoquinolin-1-yl)-2- fluoro-N-(piperidin-3-yl)benzamide | 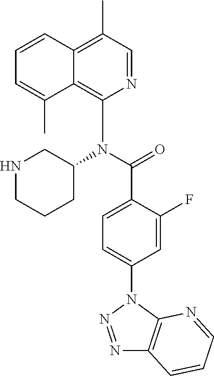 [SMILES: Cc1cnc(N(C(=O)c2ccc(-n3nnc4cccnc43)cc2F)[C@@H]2CCCNC2)c2c(C)cccc12] |
| 253 | (R)-4-(3H-[1,2,3]triazolo[4,5-b]pyridin-3- yl)-N-(5,8-dimethylisoquinolin-1-yl)-2- fluoro-N-(piperidin-3-yl)benzamide | 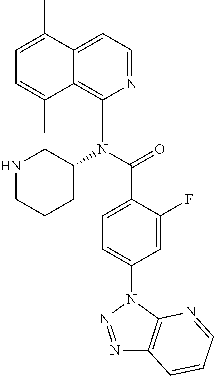 [SMILES: Cc1ccc(C)c2c(N(C(=O)c3ccc(-n4nnc5cccnc54)cc3F)[C@@H]3CCCNC3)nccc12] |
| 254 | (R)-4-(3H-[1,2,3]triazolo[4,5-b]pyridin-3- yl)-N-(6,8-dimethylisoquinolin-1-yl)-2- fluoro-N-(piperidin-3-yl)benzamide | 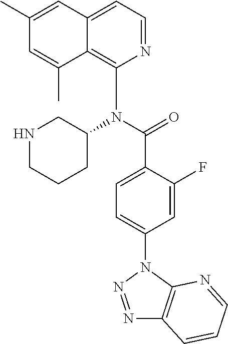 [SMILES: Cc1cc(C)c2c(N(C(=O)c3ccc(-n4nnc5cccnc54)cc3F)[C@@H]3CCCNC3)nccc2c1] |
| 255 | (R)-4-(3H-[1,2,3]triazolo[4,5-b]pyridin-3- yl)-N-(1,3-dimethyl-1H-pyrrolo[2,3- c]pyridin-7-yl)-2-fluoro-N-(piperidin-3- yl)benzamide | 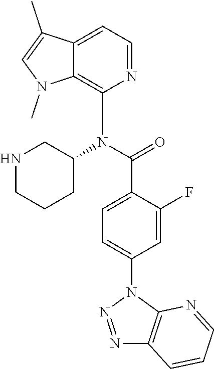 [SMILES: Cc1cn(C)c2c(N(C(=O)c3ccc(-n4nnc5cccnc54)cc3F)[C@@H]3CCCNC3)nccc12] |
| 256 | N-(isoquinolin-3-yl)-4-(1- methylpyrrolidin-3-yl)-N-((R)-piperidin-3- yl)piperidine-1-carboxamide | 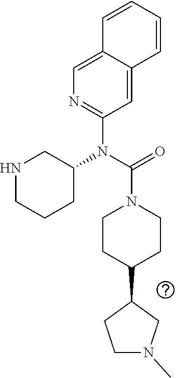 [SMILES: CN1CC[C@@H](C2CCN(C(=O)N(c3cc4ccccc4cn3)[C@@H]3CCCNC3)CC2)C1] |
| 257 | (R)-4-(6-fluorobenzo[d]isoxazol-3-yl)-N- (8-methylisoquinolin-1-yl)-N-(piperidin-3- yl)-3,6-dihydropyridine-1(2H)- carboxamide | 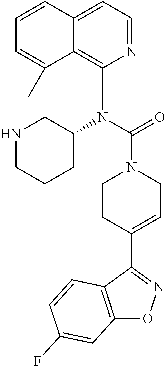 [SMILES: Cc1cccc2ccnc(N(C(=O)N3CC=C(c4noc5cc(F)ccc45)CC3)[C@@H]3CCCNC3)c12] |
| 258 | N2,N2-dimethyl-N1-(8-methylisoquinolin- 1-yl)-N1-((R)-piperidin-3-yl)piperidine- 1,2-dicarboxamide | 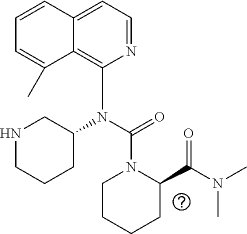 [SMILES: Cc1cccc2ccnc(N(C(=O)N3CCCC[C@@H]3C(=O)N(C)C)[C@@H]3CCCNC3)c12] |
| 259 | 2-(benzo[d]thiazol-2-yl)-N-(8- methylisoquinolin-1-yl)-N-((R)-piperidin- 3-yl)piperidine-1-carboxamide | 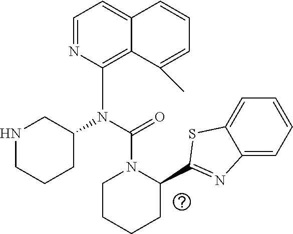 [SMILES: Cc1cccc2ccnc(N(C(=O)N3CCCC[C@@H]3c3nc4ccccc4s3)[C@@H]3CCCNC3)c12] |
| 260 | 3-(cyclobutylmethyl)-N-(8- methylisoquinolin-1-yl)-N-((R)-piperidin- 3-yl)piperidine-1-carboxamide | 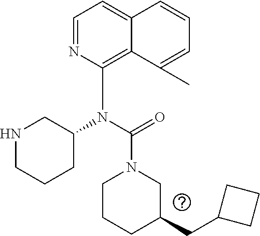 [SMILES: Cc1cccc2ccnc(N(C(=O)N3CCC[C@H](CC4CCC4)C3)[C@@H]3CCCNC3)c12] |
| 261 | 4-cyclobutyl-N-(8-methylisoquinolin-1- yl)-N-((R)-piperidin-3-yl)azepane-1- carboxamide | 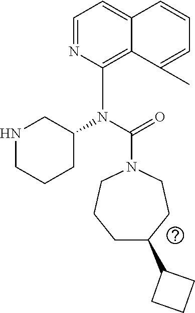 [SMILES: Cc1cccc2ccnc(N(C(=O)N3CCC[C@H](C4CCC4)CC3)[C@@H]3CCCNC3)c12] |
| 262 | (R)-4-(3-cyclopropyl-1,2,4-oxadiazol-5- yl)-N-(8-methylisoquinolin-1-yl)-N- (piperidin-3-yl)piperidine-1-carboxamide | 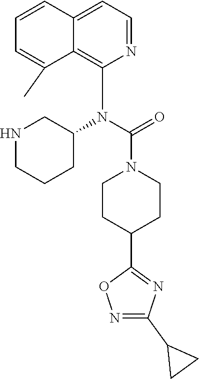 [SMILES: Cc1cccc2ccnc(N(C(=O)N3CCC(c4nc(C5CC5)no4)CC3)[C@@H]3CCCNC3)c12] |
| 263 | (R)-3-(N-(8-methylisoquinolin-1-yl)-4-(3- phenyl-1,2,4-oxadiazol-5-yl)piperidine-1- carboxamido)piperidine-1-carboxylate | 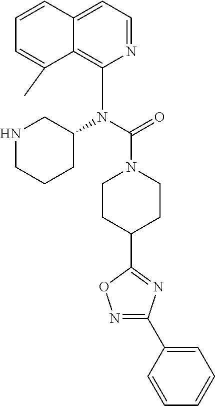 [SMILES: Cc1cccc2ccnc(N(C(=O)N3CCC(c4nc(-c5ccccc5)no4)CC3)[C@@H]3CCCNC3)c12] |
| 264 | (R)-4-(benzo[d]isothiazol-3-yl)-N-(8- methylisoquinolin-1-yl)-N-(piperidin-3- yl)piperazine-1-carboxamide | 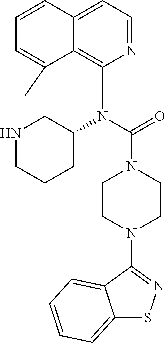 [SMILES: Cc1cccc2ccnc(N(C(=O)N3CCN(c4nsc5ccccc45)CC3)[C@@H]3CCCNC3)c12] |
| 265 | (R)-N-(8-methylisoquinolin-1-yl)-N- (piperidin-3-yl)-4′,5′- dihydrospiro[piperidine-4,7′-thieno[2,3- c]pyran]-1-carboxamide | 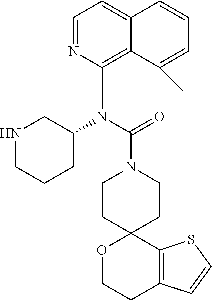 [SMILES: Cc1cccc2ccnc(N(C(=O)N3CCC4(CC3)OCCc3ccsc34)[C@@H]3CCCNC3)c12] |
| 266 | (R)-4-(1H-indazol-1-yl)-N-(8- methylisoquinolin-1-yl)-N-(piperidin-3- yl)piperidine-1-carboxamide | 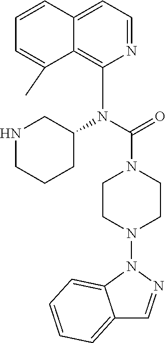 [SMILES: Cc1cccc2ccnc(N(C(=O)N3CCN(n4ncc5ccccc54)CC3)[C@@H]3CCCNC3)c12] |
| 267 | 3-methyl-N-(8-methylisoquinolin-1-yl)-3- phenyl-N-((R)-piperidin-3-yl)piperidine-1- carboxamide | 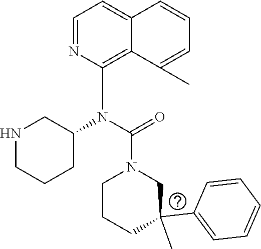 [SMILES: Cc1cccc2ccnc(N(C(=O)N3CCC[C@@](C)(c4ccccc4)C3)[C@@H]3CCCNC3)c12] |
| 268 | (R)-N-(8-methylisoquinolin-1-yl)-4-(2- oxoquinolin-1(2H)-yl)-N-(piperidin-3- yl)piperidine-1-carboxamide | 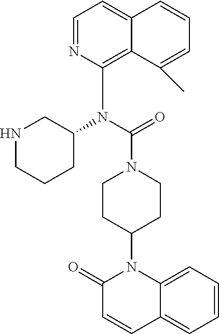 [SMILES: Cc1cccc2ccnc(N(C(=O)N3CCC(n4c(=O)ccc5ccccc54)CC3)[C@@H]3CCCNC3)c12] |
| 269 | (R)-4-(3-hydroxyphenyl)-N-(8- methylisoquinolin-1-yl)-N-(piperidin-3- yl)piperidine-1-carboxamide | 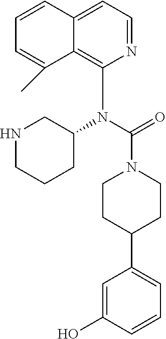 [SMILES: Cc1cccc2ccnc(N(C(=O)N3CCC(c4cccc(O)c4)CC3)[C@@H]3CCCNC3)c12] |
| 270 | (R)-N-(8-methylisoquinolin-1-yl)-4- (phthalazin-1-yl)-N-(piperidin-3- yl)piperidine-1-carboxamide | 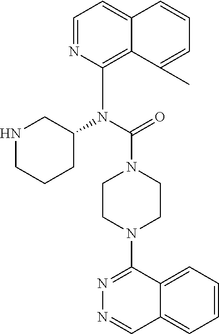 [SMILES: Cc1cccc2ccnc(N(C(=O)N3CCN(c4nncc5ccccc45)CC3)[C@@H]3CCCNC3)c12] |
| 271 | (R)-3-((1H-1,2,3-triazol-1-yl)methyl)-2- fluoro-N-(8-methylisoquinolin-1-yl)-N- (piperidin-3-yl)benzamide | 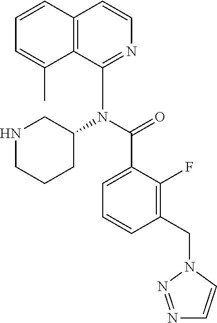 [SMILES: Cc1cccc2ccnc(N(C(=O)c3cccc(Cn4ccnn4)c3F)[C@@H]3CCCNC3)c12] |
| 272 | (R)-N-(8-chloroisoquinolin-1-yl)-2-fluoro- N-(piperidin-3-yl)-4-(pyrimidin-2- ylamino)benzamide | 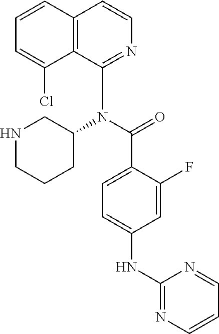 [SMILES: O=C(c1ccc(Nc2ncccn2)cc1F)N(c1nccc2cccc(Cl)c12)[C@@H]1CCCNC1] |
| 273 | (R)-3-((2H-[1,2,3]triazolo[4,5-b]pyridin-2- yl)methyl)-2-fluoro-N-(8- methylisoquinolin-1-yl)-N-(piperidin-3- yl)benzamide | 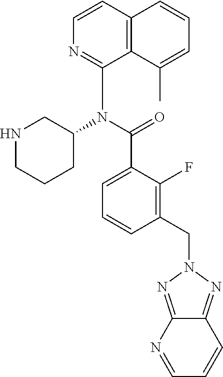 [SMILES: Cc1cccc2ccnc(N(C(=O)c3cccc(Cn4nc5cccnc5n4)c3F)[C@@H]3CCCNC3)c12] |
| 274 | (R)-4-((3-aminopyridin-2-yl)amino)-2- fluoro-N-(8-methylisoquinolin-1-yl)-N- (piperidin-3-yl)benzamide | 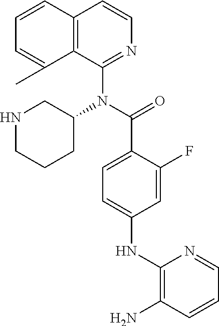 [SMILES: Cc1cccc2ccnc(N(C(=O)c3ccc(Nc4ncccc4N)cc3F)[C@@H]3CCCNC3)c12] |
| 275 | (R)-2-fluoro-N-(8-methylisoquinolin-1-yl)- N-(piperidin-3-yl)-4-(thiazol-2- ylamino)benzamide | 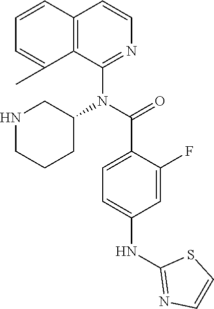 [SMILES: Cc1cccc2ccnc(N(C(=O)c3ccc(Nc4nccs4)cc3F)[C@@H]3CCCNC3)c12] |
| 276 | (R)-4-((1,7-naphthyridin-8-yl)amino)-2- fluoro-N-(8-methylisoquinolin-1-yl)-N- (piperidin-3-yl)benzamide | 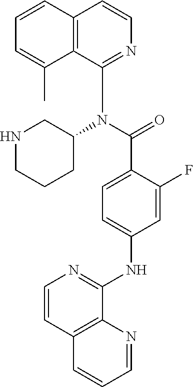 [SMILES: Cc1cccc2ccnc(N(C(=O)c3ccc(Nc4nccc5cccnc45)cc3F)[C@@H]3CCCNC3)c12] |
| 277 | (R)-2-fluoro-4-((1-methyl-1H-pyrazol-3- yl)amino)-N-(8-methylisoquinolin-1-yl)-N- (piperidin-3-yl)benzamide | 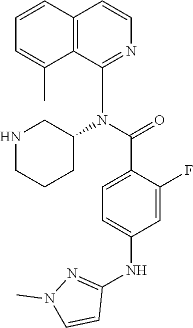 [SMILES: Cc1cccc2ccnc(N(C(=O)c3ccc(Nc4ccn(C)n4)cc3F)[C@@H]3CCCNC3)c12] |
| 278 | (R)-N-(6,8-dimethylisoquinolin-1-yl)-4-(3- methylisoxazol-5-yl)-N-(piperidin-3- yl)piperidine-1-carboxamide | 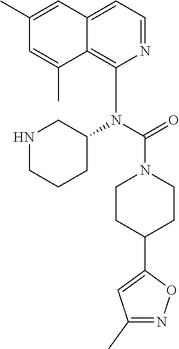 [SMILES: Cc1cc(C)c2c(N(C(=O)N3CCC(c4cc(C)no4)CC3)[C@@H]3CCCNC3)nccc2c1] |
| 279 | ethyl (R)-5-(1-((8-methylisoquinolin-1- yl)(piperidin-3-yl)carbamoyl)piperidin-4- yl)isoxazole-3-carboxylate | 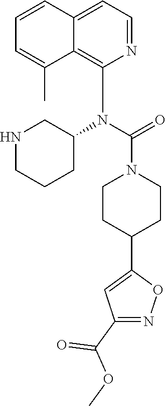 [SMILES: COC(=O)c1cc(C2CCN(C(=O)N(c3nccc4cccc(C)c34)[C@@H]3CCCNC3)CC2)on1] |
| 280 | 2-fluoro-N-(8-methyl-1-isoquinolyl) -N- [(3R)-3- piperidyl]-4-(1,2,4-thiadiazol-5- ylamino)benzamide | 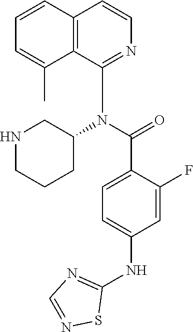 [SMILES: Cc1cccc2ccnc(N(C(=O)c3ccc(Nc4ncns4)cc3F)[C@@H]3CCCNC3)c12] |
| 281 | 2-fluoro-N-(8-methyl-1-isoquinolyl)-N- [(3R)-3-piperidyl]-4-[[5-(trifluoromethyl)- 1, 3, 4-thiadiazol-2-yl] amino] benzamide | 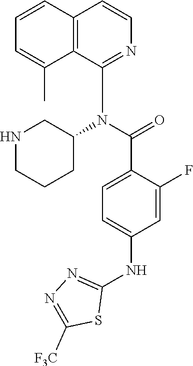 [SMILES: Cc1cccc2ccnc(N(C(=O)c3ccc(Nc4nnc(C(F)(F)F)s4)cc3F)[C@@H]3CCCNC3)c12] |
| 282 | (R)-3-((2H-1,2,3-triazol-2-yl)methyl)-2- fluoro-N-(8-methylisoquinolin-1-yl)-N- (piperidin-3-yl)benzamide | 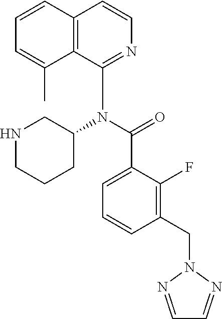 [SMILES: Cc1cccc2ccnc(N(C(=O)c3cccc(Cn4nccn4)c3F)[C@@H]3CCCNC3)c12] |
| 283 | (R)-4-(3H-[1,2,3]triazolo[4,5-b]pyridin-3- yl)-N-(7-bromoisoquinolin-1-yl)-2-fluoro- N-(piperidin-3-yl)benzamide | 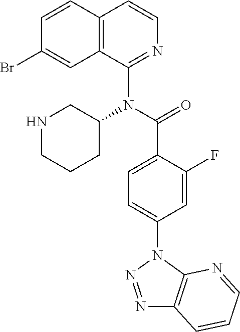 [SMILES: O=C(c1ccc(-n2nnc3cccnc32)cc1F)N(c1nccc2ccc(Br)cc12)[C@@H]1CCCNC1] |
| 284 | (R)-4-(3H-[1,2,3]triazolo[4,5-b]pyridin-3- yl)-N-(3-chloro-4-methylpyridin-2-yl)-2- fluoro-N-(piperidin-3-yl)benzamide | 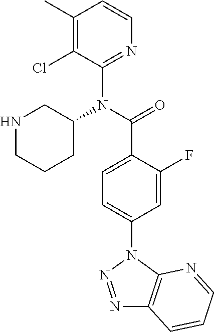 [SMILES: Cc1ccnc(N(C(=O)c2ccc(-n3nnc4cccnc43)cc2F)[C@@H]2CCCNC2)c1Cl] |
| 285 | (R)-2-fluoro-4-(5-isopropyl-2H-tetrazol-2- yl)-N-(8-methylisoquinolin-1-yl)-N- (piperidin-3-yl) benzamide | 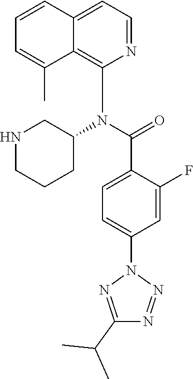 [SMILES: Cc1cccc2ccnc(N(C(=O)c3ccc(-n4nnc(C(C)C)n4)cc3F)[C@@H]3CCCNC3)c12] |
| 286 | (R)-2-fluoro-N-(8-methylisoquinolin-1-yl)- N-(piperidin-3-yl)-4-(5-propyl-2H-tetrazol- 2-yl) benzamide | 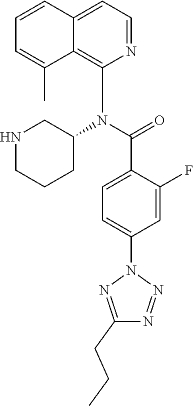 [SMILES: CCCc1nnn(-c2ccc(C(=O)N(c3nccc4cccc(C)c34)[C@@H]3CCCNC3)c(F)c2)n1] |
| 287 | (R)-N-(8-methylisoquinolin-1-yl)-4- phenyl-N-(piperidin-3-yl)-3,6- dihydropyridine-1(2H)-carboxamide | 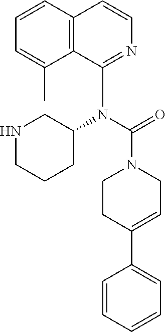 [SMILES: Cc1cccc2ccnc(N(C(=O)N3CC=C(c4ccccc4)CC3)[C@@H]3CCCNC3)c12] |
| 288 | (R)-5-(1-((8-methylisoquinolin-1- yl)(piperidin-3-yl)carbamoyl)piperidin-4- yl)isoxazole-3-carboxamide | 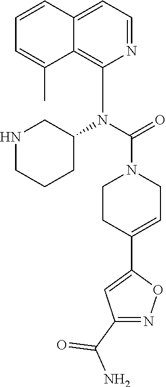 [SMILES: Cc1cccc2ccnc(N(C(=O)N3CC=C(c4cc(C(N)=O)no4)CC3)[C@@H]3CCCNC3)c12] |
| 289 | ((R,E)-4-(3H-[1,2,3]triazolo[4,5-b]pyridin- 3-yl)-2-fluoro-N-(3-(3-hydroxyprop-1-en- 1-yl)pyridin-2-yl)-N-(piperidin-3- yl)benzamide | 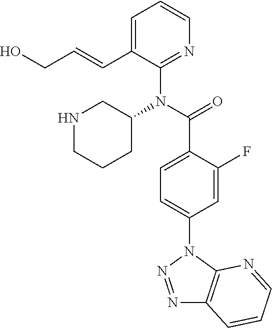 [SMILES: O=C(c1ccc(-n2nnc3cccnc32)cc1F)N(c1ncccc1/C=C/CO)[C@@H]1CCCNC1] |
| 290 | ((R)-2-fluoro-N-(8-methylisoquinolin-1- yl)-4-(oxazol-2-ylamino)-N-(piperidin-3- yl) benzamide | 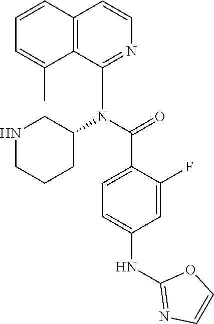 [SMILES: Cc1cccc2ccnc(N(C(=O)c3ccc(Nc4ncco4)cc3F)[C@@H]3CCCNC3)c12] |
| 291 | (R)-2-fluoro-N-(isoquinolin-3-yl)-4- (isoquinolin-5-yl)-N-(piperidin-3- yl)benzamide | 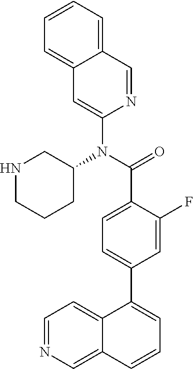 [SMILES: O=C(c1ccc(-c2cccc3cnccc23)cc1F)N(c1cc2ccccc2cn1)[C@@H]1CCCNC1] |
| 292 | (R)-2-fluoro-N-(8-methylisoquinolin-1-yl)- 4-((6-oxo-1,6-dihydropyridin-2-yl)amino)- N-(piperidin-3-yl)benzamide | 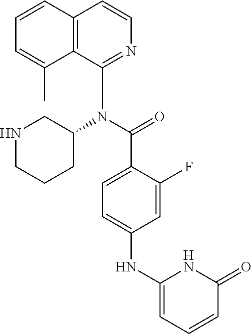 [SMILES: Cc1cccc2ccnc(N(C(=O)c3ccc(Nc4cccc(=O)[nH]4)cc3F)[C@@H]3CCCNC3)c12] |
| 293 | (R)-3-(3-fluoro-4-((8-methylisoquinolin-1- yl)(piperidin-3-yl)carbamoyl)phenyl)-3H- [1,2,3]triazolo[4,5-b]pyridine-5-carboxylic acid | 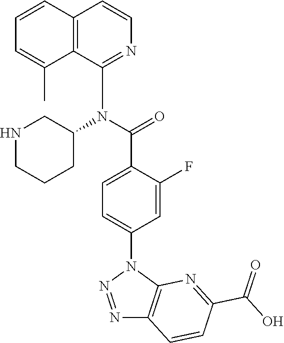 [SMILES: Cc1cccc2ccnc(N(C(=O)c3ccc(-n4nnc5ccc(C(=O)O)nc54)cc3F)[C@@H]3CCCNC3)c12] |
| 294 | 2-methyl-4-[4-[(8-methyl-1-isoquinolyl)- [(3R)-3-piperidyl] carbamoyl] phenyl] pyrazole-3-carboxylic acid | 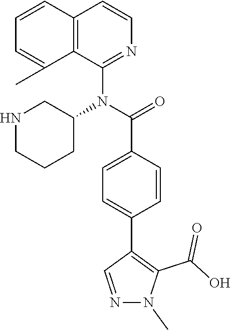 [SMILES: Cc1cccc2ccnc(N(C(=O)c3ccc(-c4cnn(C)c4C(=O)O)cc3)[C@@H]3CCCNC3)c12] |
| 295 | 2-methyl-4-[4-[(8-methyl-1-isoquinolyl)- [(3R)-3-piperidyl] carbamoyl] phenyl] pyrazole-3-carboxylic acid | 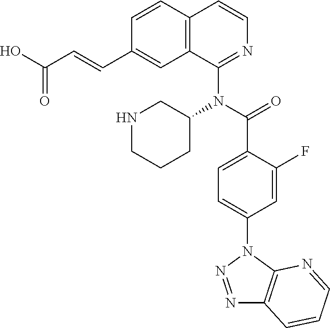 [SMILES: O=C(O)/C=C/c1ccc2ccnc(N(C(=O)c3ccc(-n4nnc5cccnc54)cc3F)[C@@H]3CCCNC3)c2c1] |
| 296 | (R)-2-fluoro-N-(8-methylisoquinolin-1-yl)- N-(piperidin-3-yl)-4-(4,5,6,7-tetrahydro- 1H-pyrazolo[4,3-c]pyridin-3-yl)benzamide | 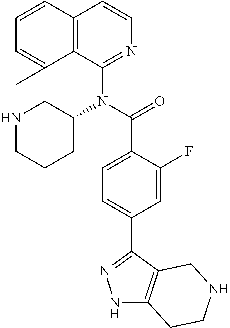 [SMILES: Cc1cccc2ccnc(N(C(=O)c3ccc(-c4n[nH]c5c4CNCC5)cc3F)[C@@H]3CCCNC3)c12] |
| 297 | (R)-4-(2-(benzyloxy)phenyl)-4-hydroxy-N- (8-methylisoquinolin-1-yl)-N-(piperidin-3- yl)piperidine-1-carboxamide | 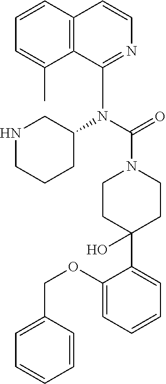 [SMILES: Cc1cccc2ccnc(N(C(=O)N3CCC(O)(c4ccccc4OCc4ccccc4)CC3)[C@@H]3CCCNC3)c12] |
| 298 | 4-(5-(1-chloroethyl)-2H-tetrazol-2-yl)-2- fluoro-N-(8-methylisoquinolin-1-yl)-N- ((R)-piperidin-3-yl) benzamide | 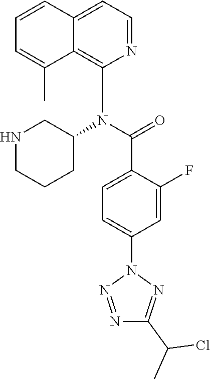 [SMILES: Cc1cccc2ccnc(N(C(=O)c3ccc(-n4nnc(C(C)Cl)n4)cc3F)[C@@H]3CCCNC3)c12] |
| 299 | (R)-4-(2-(benzyloxy)phenyl)-N-(8- methylisoquinolin-1-yl)-N-(piperidin-3- yl)-3,6-dihydropyridine-1(2H)- carboxamide | 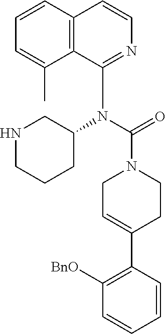 [SMILES: Cc1cccc2ccnc(N(C(=O)N3CC=C(c4ccccc4OCc4ccccc4)CC3)[C@@H]3CCCNC3)c12] |
| 300 | (R)-4-(3H-[1,2,3]triazolo[4,5-b]pyridin-3- yl)-2-fluoro-N-(2,7-naphthyridin-1-yl)-N- (piperidin-3-yl)benzamide | 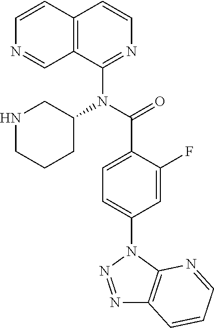 [SMILES: O=C(c1ccc(-n2nnc3cccnc32)cc1F)N(c1nccc2ccncc12)[C@@H]1CCCNC1] |
| 301 | (R)-1-formyl-N-(8-methylisoquinolin-1- yl)-N-(piperidin-3-yl) piperidine-4- carboxamide | 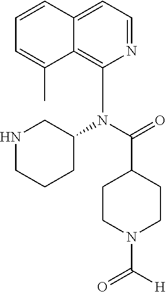 [SMILES: Cc1cccc2ccnc(N(C(=O)C3CCN(C=O)CC3)[C@@H]3CCCNC3)c12] |
| 302 | (R)-N-(isoquinolin-1-yl)-N-(piperidin-3- yl)-4-(pyrazolo[1,5-a]pyrimidin-3- yl)benzamide | 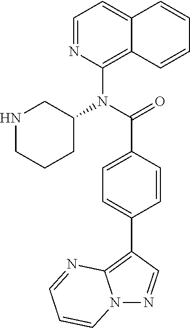 [SMILES: O=C(c1ccc(-c2cnn3cccnc23)cc1)N(c1nccc2ccccc12)[C@@H]1CCCNC1] |
| 303 | (R)-N-(1-methyl-1H-pyrrolo[2,3-c]pyridin- 7-yl)-N-(piperidin-3-yl)-4-(pyrazolo[1,5- alpyrimidin-3-yl)benzamide | 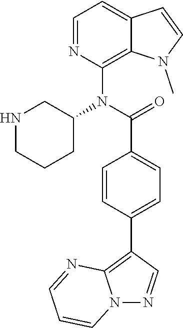 [SMILES: Cn1ccc2ccnc(N(C(=O)c3ccc(-c4cnn5cccnc45)cc3)[C@@H]3CCCNC3)c21] |
| 304 | (R,E)-4-(3-(4-(dimethylamino)but-2- enamido)phenyl)-N-(1-methyl-1H- pyrrolo[2,3-c]pyridin-7-yl)-N-(piperidin-3- yl)piperidine-1-carboxamide | 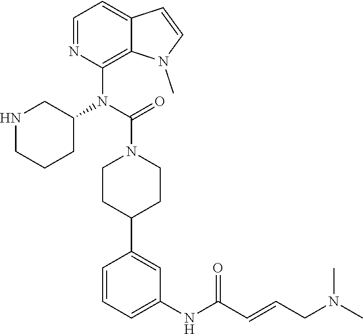 [SMILES: CN(C)C/C=C/C(=O)Nc1cccc(C2CCN(C(=O)N(c3nccc4ccn(C)c34)[C@@H]3CCCNC3)CC2)c1] |
| 305 | (R)-4-(3H-[1,2,3]triazolo[4,5-b]pyridin-3- yl)-N-(3-chloropyridin-2-yl)-N-(piperidin- 3-yl)benzamide | 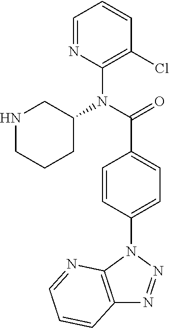 [SMILES: O=C(c1ccc(-n2nnc3cccnc32)cc1)N(c1ncccc1Cl)[C@@H]1CCCNC1] |
| 306 | (R)-4-(3H-[1,2,3]triazolo[4,5-b]pyridin-3- yl)-2-methyl-N-(8-methylisoquinolin-1- yl)-N-(piperidin-3-yl)benzamide | 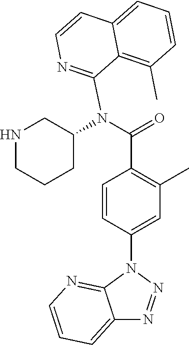 [SMILES: Cc1cc(-n2nnc3cccnc32)ccc1C(=O)N(c1nccc2cccc(C)c12)[C@@H]1CCCNC1] |
| 307 | (R)-4-(3H-[1,2,3]triazolo[4,5-b]pyridin-3- yl)-2-ethyl-N-(8-methylisoquinolin-1-yl)- N-(piperidin-3-yl)benzamide | 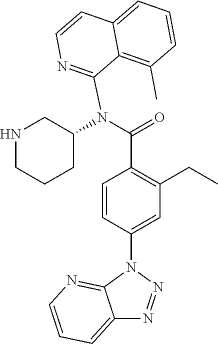 [SMILES: CCc1cc(-n2nnc3cccnc32)ccc1C(=O)N(c1nccc2cccc(C)c12)[C@@H]1CCCNC1] |
| 308 | (R)-4-(3H-[1,2,3]triazolo[4,5-b]pyridin-3- yl)-2-methoxy-N-(8-methylisoquinolin-1- yl)-N-(piperidin-3-yl)benzamide | 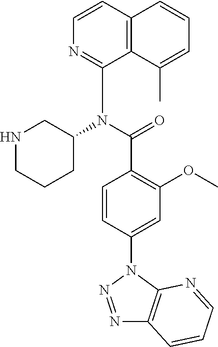 [SMILES: COc1cc(-n2nnc3cccnc32)ccc1C(=O)N(c1nccc2cccc(C)c12)[C@@H]1CCCNC1] |
| 309 | (R)-N-(8-methylisoquinolin-1-yl)-N- (piperidin-3-yl)-4-(pyrimidin-2- ylamino)benzamide | 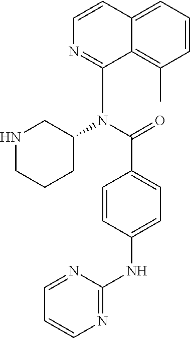 [SMILES: Cc1cccc2ccnc(N(C(=O)c3ccc(Nc4ncccn4)cc3)[C@@H]3CCCNC3)c12] |
| 310 | (R)-4-(3H- [1,2,3] triazolo[4,5-b] pyridin- 3-yl)-2-chloro-N-(8-methylisoquinolin-1- yl)-N-(piperidin-3-yl) benzamide | 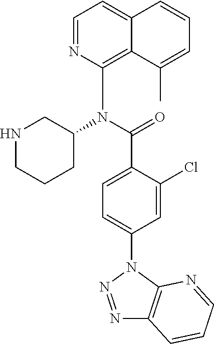 [SMILES: Cc1cccc2ccnc(N(C(=O)c3ccc(-n4nnc5cccnc54)cc3Cl)[C@@H]3CCCNC3)c12] |
| 311 | (R)-2-fluoro-N-(8-methylisoquinolin-1-yl)- 4-morpholino-N-(piperidin-3-yl)benzamide | 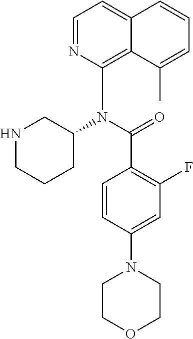 [SMILES: Cc1cccc2ccnc(N(C(=O)c3ccc(N4CCOCC4)cc3F)[C@@H]3CCCNC3)c12] |
| 312 | (R)-4-(3H-[1,2,3]triazolo[4,5-b]pyridin-3- yl)-N-(8-methylisoquinolin-1-yl)-N- (piperidin-3-yl)-2- (trifluoromethyl)benzamide | 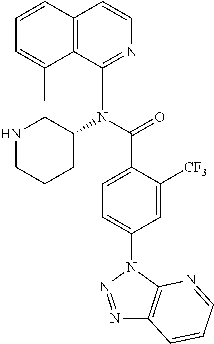 [SMILES: Cc1cccc2ccnc(N(C(=O)c3ccc(-n4nnc5cccnc54)cc3C(F)(F)F)[C@@H]3CCCNC3)c12] |
| 313 | (R)-4-(3H-[1,2,3]triazolo[4,5-b]pyridin-3- yl)-2-fluoro-N-(isoquinolin-1-yl)-N- (piperidin-3-yl)benzamide | 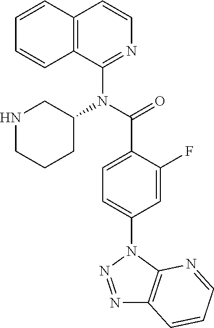 [SMILES: O=C(c1ccc(-n2nnc3cccnc32)cc1F)N(c1nccc2ccccc12)[C@@H]1CCCNC1] |
| 314 | (R)-2-fluoro-N-(8-methylisoquinolin-1-yl)- N-(piperidin-3-yl)-4-(thiazol-5-yl) benzamide | 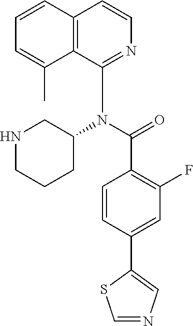 [SMILES: Cc1cccc2ccnc(N(C(=O)c3ccc(-c4cncs4)cc3F)[C@@H]3CCCNC3)c12] |
| 315 | (R)-2-fluoro-N-(8-methylisoquinolin-1-yl)- 4-(4-methylthiazol-2-yl)-N-(piperidin-3- yl)benzamide | 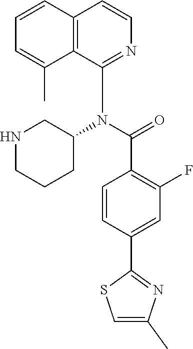 [SMILES: Cc1csc(-c2ccc(C(=O)N(c3nccc4cccc(C)c34)[C@@H]3CCCNC3)c(F)c2)n1] |
| 316 | (R)-2-fluoro-N-(8-methylisoquinolin-1-yl)- 4-(3-methylisoxazol-5-yl)-N-(piperidin-3- yl) benzamide | 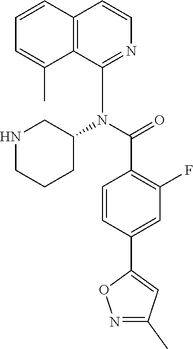 [SMILES: Cc1cc(-c2ccc(C(=O)N(c3nccc4cccc(C)c34)[C@@H]3CCCNC3)c(F)c2)on1] |
| 317 | (R)-4-(3H-[1,2,3]triazolo[4,5-b]pyridin-3- yl)-2-fluoro-N-(1-methyl-1H-pyrrolo[2,3- c]pyridin-7-yl)-N-(piperidin-3- yl)benzamide | 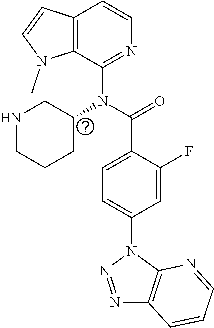 [SMILES: Cn1ccc2ccnc(N(C(=O)c3ccc(-n4nnc5cccnc54)cc3F)[C@@H]3CCCNC3)c21] |
| 318 | (R)-6-(3-fluoro-4-((8-methylisoquinolin-1- yl)(piperidin-3-yl)carbamoyl) phenyl) pyrazine-2-carboxamide | 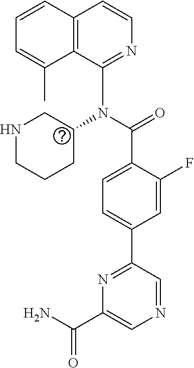 [SMILES: Cc1cccc2ccnc(N(C(=O)c3ccc(-c4cncc(C(N)=O)n4)cc3F)[C@@H]3CCCNC3)c12] |
| 319 | (R)-2-fluoro-N-(8-methylisoquinolin-1-yl)- 4-((4-methylpyrimidin-2-yl)amino)-N- (piperidin-3-yl)benzamide | 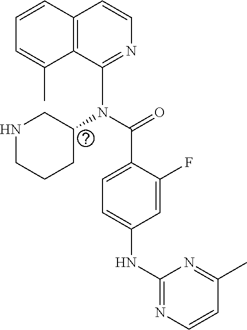 [SMILES: Cc1ccnc(Nc2ccc(C(=O)N(c3nccc4cccc(C)c34)[C@@H]3CCCNC3)c(F)c2)n1] |
| 320 | N-[8-[(E)-3-amino-3-oxo-prop-1-enyl]-1- isoquinolyl]-2-fluoro-N-[(3R)-3-piperidyl]- 4-(triazolo[4,5-b]pyridin-3-yl)benzamide | 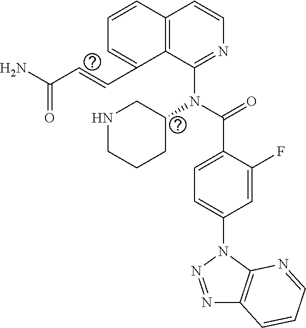 [SMILES: NC(=O)/C=C/c1cccc2ccnc(N(C(=O)c3ccc(-n4nnc5cccnc54)cc3F)[C@@H]3CCCNC3)c12] |
| 321 | (R)-2-fluoro-4-((4- (hydroxymethyl)pyrimidin-2-yl)amino)-N- (8-methylisoquinolin-1-yl)-N-(piperidin-3- yl)benzamide | 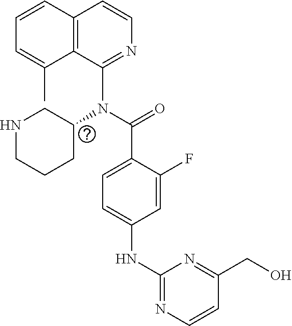 [SMILES: Cc1cccc2ccnc(N(C(=O)c3ccc(Nc4nccc(CO)n4)cc3F)[C@@H]3CCCNC3)c12] |
| 322 | (R)-2-fluoro-4-((5- (hydroxymethyl)pyrimidin-2-yl)amino)-N- (8-methylisoquinolin-1-yl)-N-(piperidin-3- yl)benzamide | 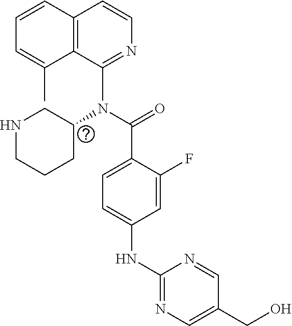 [SMILES: Cc1cccc2ccnc(N(C(=O)c3ccc(Nc4ncc(CO)cn4)cc3F)[C@@H]3CCCNC3)c12] |
| 323 | (R)-2-fluoro-4-(3-methyl-1H-pyrazol-5- yl)-N-(8-methylisoquinolin-1-yl)-N- (piperidin-3-yl) benzamide | 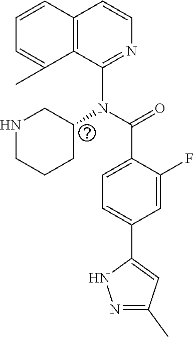 [SMILES: Cc1cc(-c2ccc(C(=O)N(c3nccc4cccc(C)c34)[C@@H]3CCCNC3)c(F)c2)[nH]n1] |
| 324 | (R)-2-fluoro-4-(3-methyl-1H-1,2,4-triazol- 5-yl)-N-(8-methylisoquinolin-1-yl)-N- (piperidin-3-yl) benzamide | 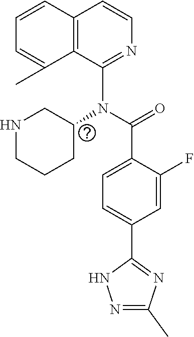 [SMILES: Cc1n[nH]c(-c2ccc(C(=O)N(c3nccc4cccc(C)c34)[C@@H]3CCCNC3)c(F)c2)n1] |
| 325 | (R)-4-(3H-[1,2,3]triazolo[4,5-b]pyridin-3- yl)-2-fluoro-N-(1,7-naphthyridin-8-yl)-N- (piperidin-3-yl)benzamide | 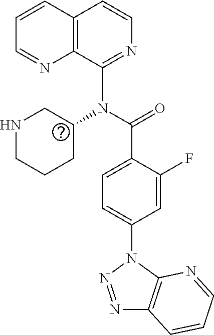 [SMILES: O=C(c1ccc(-n2nnc3cccnc32)cc1F)N(c1nccc2cccnc12)[C@@H]1CCCNC1] |
| 326 | (R)-3,4-bis(1-methyl-1H-pyrazol-4-yl)-N- (8-methylisoquinolin-1-yl)-N-(piperidin-3- yl) benzamide | 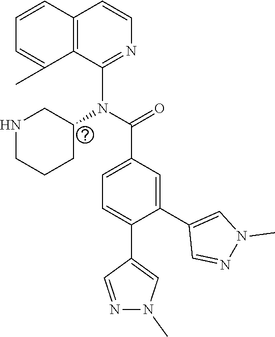 [SMILES: Cc1cccc2ccnc(N(C(=O)c3ccc(-c4cnn(C)c4)c(-c4cnn(C)c4)c3)[C@@H]3CCCNC3)c12] |
| 327 | 4-cyano-N-(8-methyl-1-isoquinolyl)-3-(4- phenylphenyl)-N-[(3R)-3- piperidyl]benzamide | 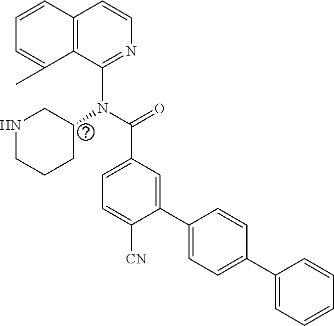 [SMILES: Cc1cccc2ccnc(N(C(=O)c3ccc(C#N)c(-c4ccc(-c5ccccc5)cc4)c3)[C@@H]3CCCNC3)c12] |
| 328 | 4-cyano-N-(8-methyl-1-isoquinolyl)-N- [(3R)-3-piperidyl]-3-[4-(3- pyridyl)phenyl]benzamide | 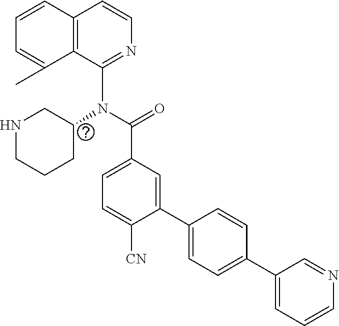 [SMILES: Cc1cccc2ccnc(N(C(=O)c3ccc(C#N)c(-c4ccc(-c5cccnc5)cc4)c3)[C@@H]3CCCNC3)c12] |
| 329 | (R)-6-cyano-N-(8-methylisoquinolin-1-yl)- N-(piperidin-3-yl)-3′-(1H-pyrazol-4-yl)- [1,1′-biphenyl]-3-carboxamide | 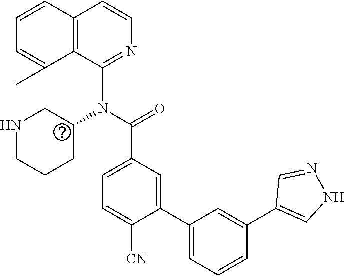 [SMILES: Cc1cccc2ccnc(N(C(=O)c3ccc(C#N)c(-c4cccc(-c5cn[nH]c5)c4)c3)[C@@H]3CCCNC3)c12] |
| 330 | 4-cyano-N-(8-methyl-1-isoquinolyl)-N- [(3R)-3-piperidyl]-3-[4-(4- pyridyl)phenyl]benzamide | 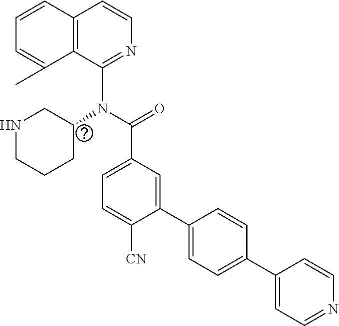 [SMILES: Cc1cccc2ccnc(N(C(=O)c3ccc(C#N)c(-c4ccc(-c5ccncc5)cc4)c3)[C@@H]3CCCNC3)c12] |
| 331 | (R)-4-(3H-[1,2,3]triazolo[4,5-b]pyridin-3- yl)-2-fluoro-N-(6-(3-hydroxyprop-1-en-1- yl)isoquinolin-1-yl)-N-(piperidin-3- yl)benzamide | 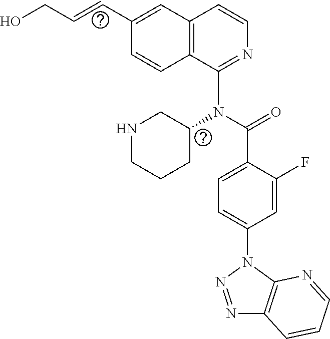 [SMILES: O=C(c1ccc(-n2nnc3cccnc32)cc1F)N(c1nccc2cc(/C=C/CO)ccc12)[C@@H]1CCCNC1] |
| 332 | (R)-N-(8-methylisoquinolin-1-yl)-4-(2- oxo-1,2-dihydropyridin-3-yl)-N-(piperidin- 3-yl)benzamide | 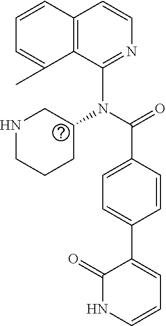 [SMILES: Cc1cccc2ccnc(N(C(=O)c3ccc(-c4ccc[nH]c4=O)cc3)[C@@H]3CCCNC3)c12] |
| 333 | 4-cyano-N-(8-methyl-1-isoquinolyl)-N- [(3R)-3-piperidyl]-2-(1H-pyrazol-5- yl)benzamide | 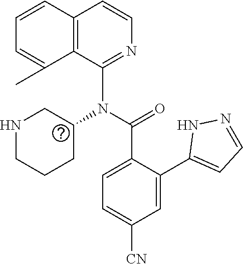 [SMILES: Cc1cccc2ccnc(N(C(=O)c3ccc(C#N)cc3-c3ccn[nH]3)[C@@H]3CCCNC3)c12] |
| 334 | 4-cyano-N-(8-methyl-1-isoquinolyl)-N- [(3R)-3-piperidyl]-2-(3-pyridyl)benzamide | 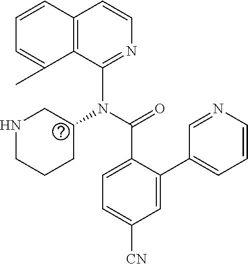 [SMILES: Cc1cccc2ccnc(N(C(=O)c3ccc(C#N)cc3-c3cccnc3)[C@@H]3CCCNC3)c12] |
| 335 | 4-cyano-N-(8-methyl-1-isoquinolyl)-N- [(3R)-3-piperidyl]-2-(2-pyridyl)benzamide | 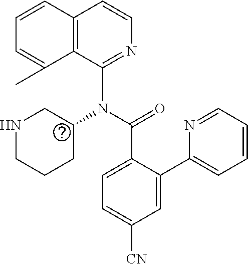 [SMILES: Cc1cccc2ccnc(N(C(=O)c3ccc(C#N)cc3-c3ccccn3)[C@@H]3CCCNC3)c12] |
| 336 | 4-cyano-N-(8-methyl-1-isoquinolyl)-N- [(3R)-3-piperidyl]-3-[3-(3- pyridyl)phenyl]benzamide | 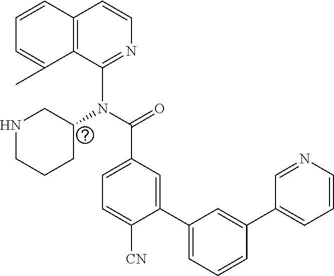 [SMILES: Cc1cccc2ccnc(N(C(=O)c3ccc(C#N)c(-c4cccc(-c5cccnc5)c4)c3)[C@@H]3CCCNC3)c12] |
| 337 | (R,Z)-4-(3H-[1,2,3]triazolo[4,5-b]pyridin- 3-yl)-2-fluoro-N-(7-(3-hydroxyprop-1-en- 1-yl)isoquinolin-1-yl)-N-(piperidin-3- yl)benzamide | 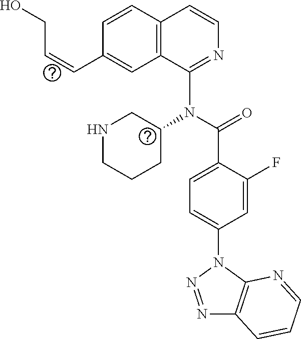 [SMILES: O=C(c1ccc(-n2nnc3cccnc32)cc1F)N(c1nccc2ccc(/C=C\CO)cc12)[C@@H]1CCCNC1] |
| 338 | (R,Z)-N-(8-(3-hydroxyprop-1-en-1- yl)isoquinolin-1-yl)-4-(1-methyl-1H-1,2,3- triazol-4-yl)-N-(piperidin-3-yl)benzamide | 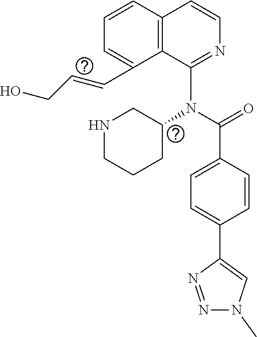 [SMILES: Cn1cc(-c2ccc(C(=O)N(c3nccc4cccc(/C=C/CO)c34)[C@@H]3CCCNC3)cc2)nn1] |
| 339 | 2-(4-((S)-1-((8-methylisoquinolin-1- yl)((R)-piperidin-3- yl)carbamoyl)piperidin-3-yl)phenyl)-2H- indazole-7-carboxamide | 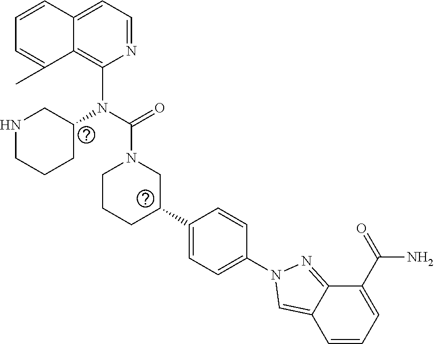 [SMILES: Cc1cccc2ccnc(N(C(=O)N3CCC[C@@H](c4ccc(-n5cc6cccc(C(N)=O)c6n5)cc4)C3)[C@@H]3CCCNC3)c12] |
| 340 | (R)-3-(4-amino-3-(4-phenoxyphenyl)-1H- pyrazolo[3,4-d]pyrimidin-1-yl)-N-(8- methylisoquinolin-1-yl)-N-((R)-piperidin- 3-yl)piperidine-1-carboxamide | 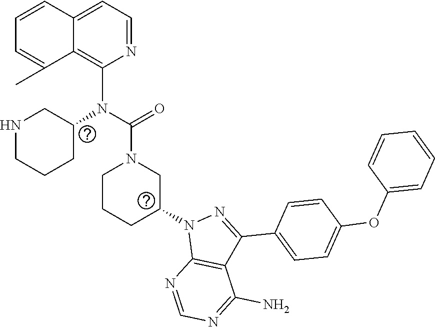 [SMILES: Cc1cccc2ccnc(N(C(=O)N3CCC[C@@H](n4nc(-c5ccc(Oc6ccccc6)cc5)c5c(N)ncnc54)C3)[C@@H]3CCCNC3)c12] |
| 341 | N-(8-methylisoquinolin-1-yl)-3-(5-phenyl- 1H-imidazol-2-yl)-N-((R)-piperidin-3- yl)piperidine-1-carboxamide | 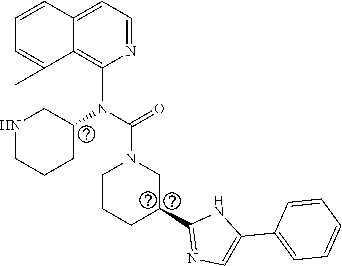 [SMILES: Cc1cccc2ccnc(N(C(=O)N3CCC[C@H](c4ncc(-c5ccccc5)[nH]4)C3)[C@@H]3CCCNC3)c12] |
| 342 | benzyl (R)-2-((8-methylisoquinolin-1-yl) (piperidin-3-yl) carbamoyl)-2,9-diazaspiro [5.5] undecane-9-carboxylate | 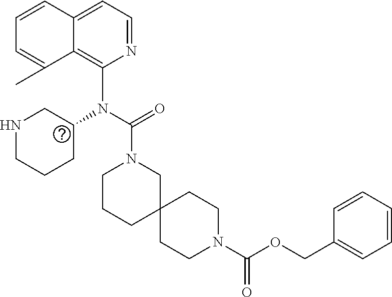 [SMILES: Cc1cccc2ccnc(N(C(=O)N3CCCC4(CCN(C(=O)OCc5ccccc5)CC4)C3)[C@@H]3CCCNC3)c12] |
| 343 | (R)-N-(8-methylisoquinolin-1-yl)-3- phenyl-N-(piperidin-3-yl)-1,4,6,7- tetrahydro-5H-pyrazolo[4,3-c]pyridine-5- carboxamide | 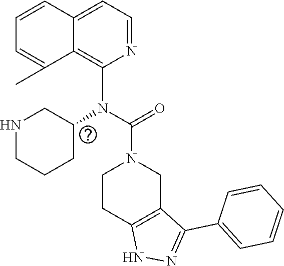 [SMILES: Cc1cccc2ccnc(N(C(=O)N3CCc4[nH]nc(-c5ccccc5)c4C3)[C@@H]3CCCNC3)c12] |
| 344 | N-(8-methylisoquinolin-1-yl)-3-(3-oxo-3- (phenylamino) propyl)-N-((R)-piperidin-3- yl) piperidine-1-carboxamide | 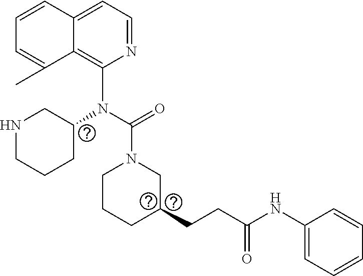 [SMILES: Cc1cccc2ccnc(N(C(=O)N3CCC[C@H](CCC(=O)Nc4ccccc4)C3)[C@@H]3CCCNC3)c12] |
| 345 | 3-(1,3-dioxoisoindolin-2-yl)-N-(8- methylisoquinolin-1-yl)-N-((R)-piperidin- 3-yl)piperidine-1-carboxamide | 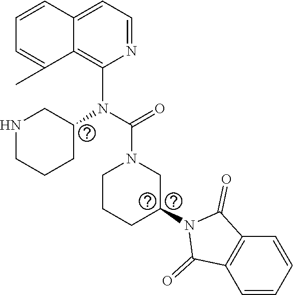 [SMILES: Cc1cccc2ccnc(N(C(=O)N3CCC[C@H](N4C(=O)c5ccccc5C4=O)C3)[C@@H]3CCCNC3)c12] |
| 346 | N-[4-[(Z)-4-hydroxybut-1-enyl]-2- pyridyl]-4-(1-methyltriazol-4-yl)-N-[(3R)- 3-piperidyl] benzamide | 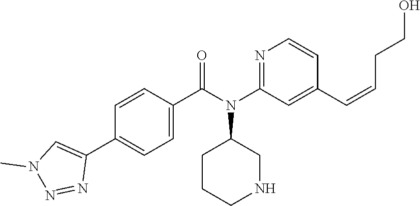 [SMILES: Cn1cc(-c2ccc(C(=O)N(c3cc(/C=C\CCO)ccn3)[C@@H]3CCCNC3)cc2)nn1] |
| 347 | N-(8-methyl-1-isoquinolyl)-4-(1- methyltriazol-4-yl)-N-[(3R)-3- piperidyl]piperidine-1-carboxamide | 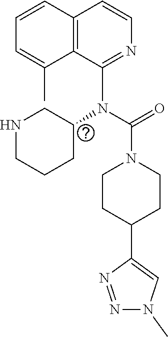 [SMILES: Cc1cccc2ccnc(N(C(=O)N3CCC(c4cn(C)nn4)CC3)[C@@H]3CCCNC3)c12] |
| 348 | (R,E)-N-(8-(2-(1H-pyrazol-4- yl)vinyl)isoquinolin-1-yl) -4-(1-methyl- 1H-1,2,3-triazol-4-yl)-N-(piperidin-3- yl)benzamide | 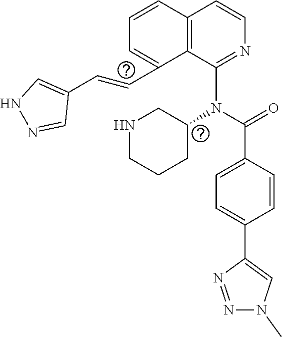 [SMILES: Cn1cc(-c2ccc(C(=O)N(c3nccc4cccc(/C=C/c5cn[nH]c5)c34)[C@@H]3CCCNC3)cc2)nn1] |
| 349 | 4-(1-methyltriazol-4-yl)-N-[(3R)-3- piperidyl]-N-[8-[(E)-2-(3-pyridyl)vinyl]-1- isoquinolyl]benzamide | 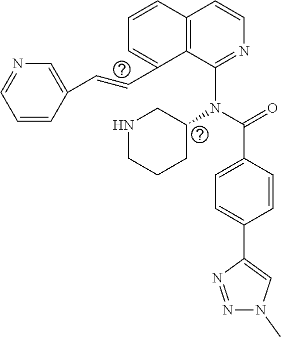 [SMILES: Cn1cc(-c2ccc(C(=O)N(c3nccc4cccc(/C=C/c5cccnc5)c34)[C@@H]3CCCNC3)cc2)nn1] |
| 353 | (R)-4-(4-aminopyrimidin-5-yl)-2-fluoro-N- (8-methylisoquinolin-1-yl)-N-(piperidin-3- yl)benzamide | 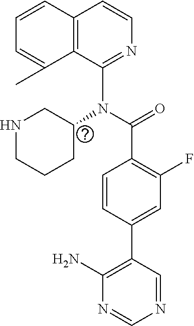 [SMILES: Cc1cccc2ccnc(N(C(=O)c3ccc(-c4cncnc4N)cc3F)[C@@H]3CCCNC3)c12] |
| 354 | 4-(4,6-dimethylpyrimidin-5-yl)-2-fluoro- N-(8-methyl-1-isoquinolyl)-N-[(3R)-3- piperidyl]benzamide | 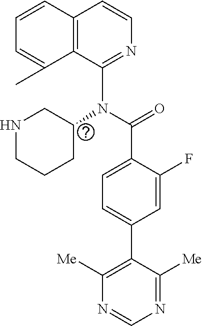 [SMILES: Cc1ncnc(C)c1-c1ccc(C(=O)N(c2nccc3cccc(C)c23)[C@@H]2CCCNC2)c(F)c1] |
| 361 | (R)-4-(4-amino-2-methylpyridin-3-yl)-2- fluoro-N-(8-methylisoquinolin-1-yl)-N- (piperidin-3-yl)benzamide | 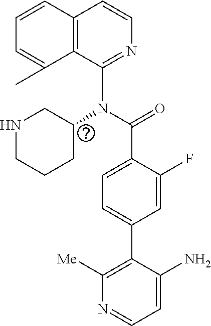 [SMILES: Cc1nccc(N)c1-c1ccc(C(=O)N(c2nccc3cccc(C)c23)[C@@H]2CCCNC2)c(F)c1] |
| 362 | 3-allyl-N-(4-allyl-2-pyridyl)-4-chloro-N- [(3R)-3-piperidyl]benzamide | 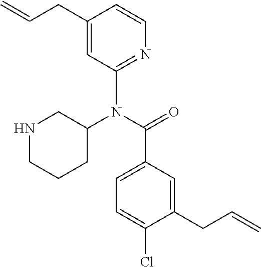 [SMILES: C=CCc1ccnc(N(C(=O)c2ccc(Cl)c(CC=C)c2)C2CCCNC2)c1] |
| 363 | 2-fluoro-4-(1-methyltriazol-4-yl)-N-[(3R)- 3-piperidyl]-N-[2-(4-pyridyl)thieno[3,2- c]pyridin-4-yl]benzamide | 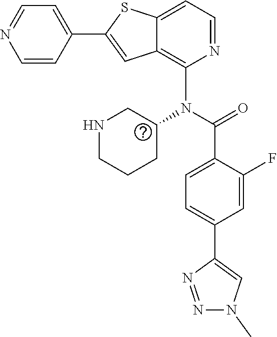 [SMILES: Cn1cc(-c2ccc(C(=O)N(c3nccc4sc(-c5ccncc5)cc34)[C@@H]3CCCNC3)c(F)c2)nn1] |
| 364 | (R)-4-(3H-[1,2,3]triazolo[4,5-b]pyridin-3- yl)-2-fluoro-N-(piperidin-3-yl)-N-(2- (pyridin-3-yl)thieno[3,2-c]pyridin-4- yl)benzamide | 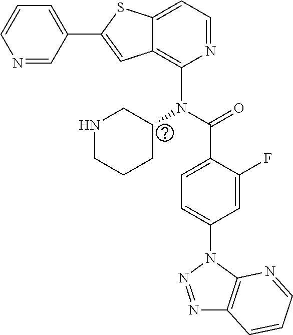 [SMILES: O=C(c1ccc(-n2nnc3cccnc32)cc1F)N(c1nccc2sc(-c3cccnc3)cc12)[C@@H]1CCCNC1] |
| 365 | (R)-4-(3H-[1,2,3]triazolo[4,5-b]pyridin-3- yl)-2-fluoro-N-(piperidin-3-yl)-N-(2- (pyridin-2-yl)thieno[3,2-c]pyridin-4- yl)benzamide | 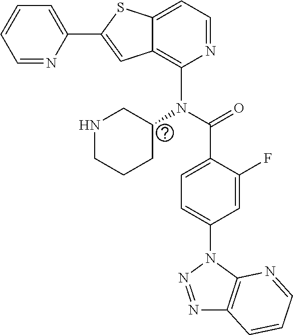 [SMILES: O=C(c1ccc(-n2nnc3cccnc32)cc1F)N(c1nccc2sc(-c3ccccn3)cc12)[C@@H]1CCCNC1] |
| 366 | (R)-4-(3H-[1,2,3]triazolo[4,5-b]pyridin-3- yl)-2-fluoro-N-(6-(oxazol-2-yl)isoquinolin- 1-yl)-N-(piperidin-3-yl)benzamide | 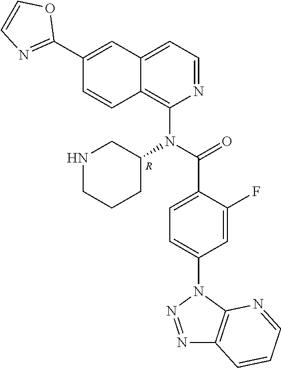 |
| 367 | (R)-4-(3H-[1,2,3]triazolo[4,5-b]pyridin-3- yl)-2-fluoro-N-(piperidin-3-yl)-N-(6- (pyridin-3-yl)isoquinolin-1-yl)benzamide | 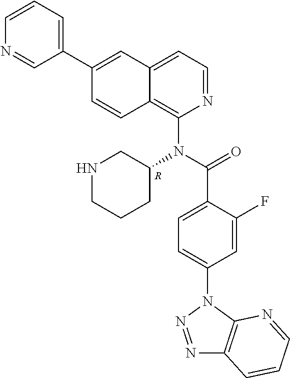 |
| 368 | (R)-4-(3H-[1,2,3]triazolo[4,5-b]pyridin-3- yl)-2-fluoro-N-(piperidin-3-yl)-N-(6- (thiophen-2-yl)isoquinolin-1-yl)benzamide | 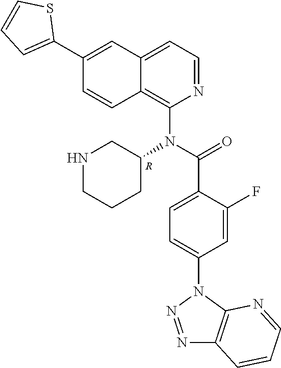 |
| 369 | (R)-1-(4-(3H-[1,2,3]triazolo[4,5-b]pyridin- 3-yl)-2-fluoro-N-(piperidin-3- yl)benzamido)-N-methylisoquinoline-6- carboxamide | 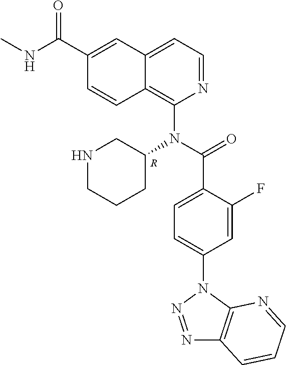 |
| 370 | (R)-2-fluoro-4-(1-methyl-1H-1,2,3-triazol- 4-yl)-N-(piperidin-3-yl)-N-(6-(pyridin-3- yl)isoquinolin-1-yl)benzamide | 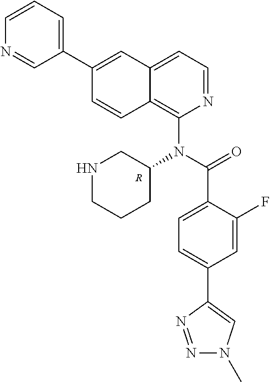 |
| 371 | (R)-1-(4-(3H-[1,2,3]triazolo[4,5-b]pyridin- 3-yl)-2-fluoro-N-(piperidin-3- yl)benzamido)-N-(2- hydroxyethyl)isoquinoline-6-carboxamide | 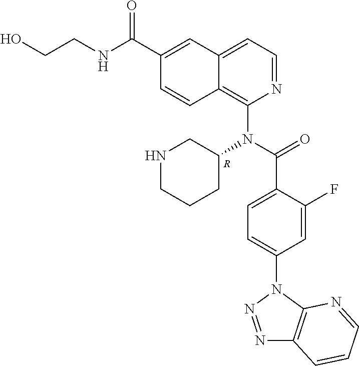 |
| 372 | (R)-1-(4-(3H-[1,2,3]triazolo[4,5-b]pyridin- 3-yl)-2-fluoro-N-(piperidin-3- yl)benzamido)-N-isopropylisoquinoline-6- carboxamide | 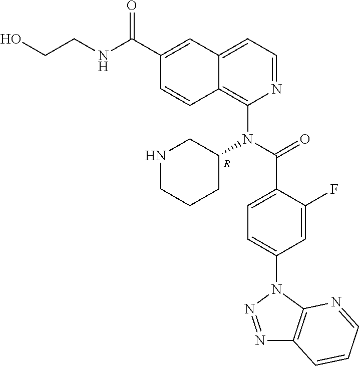 |
| 373 | (R)-1-(4-(3H-[1,2,3]triazolo[4,5-b]pyridin- 3-yl)-2-fluoro-N-(piperidin-3- yl)benzamido)-N-benzylisoquinoline-6- carboxamide | 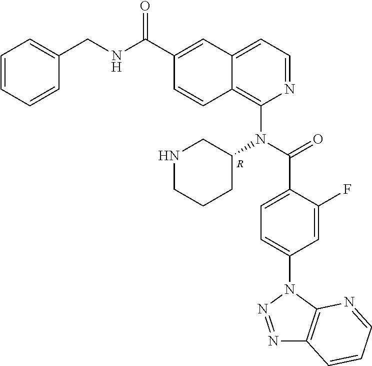 |
| 374 | 4-[4-[[2-fluoro-4-(triazolo[4,5-b]pyridin-3- yl)benzoyl]-[(3R)-3-piperidyl]amino] thieno [3,2-c]pyridin-2-yl]-N-methyl- pyridine-2-carboxamide | 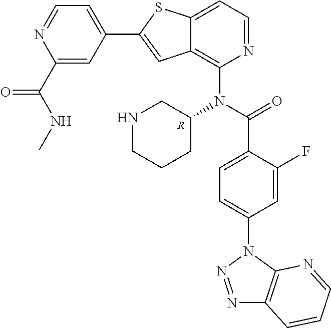 |
| 375 | methyl 4-[4-[[2-fluoro-4-(triazolo[4,5- b]pyridin-3-yl)benzoyl]-[(3R)-3- piperidyl]amino]thieno[3,2-c]pyridin-2- yl]benzoate | 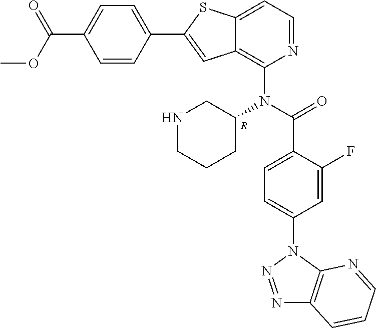 |
| 376 | 2-fluoro-N-[2-(3-hydroxy-3-methyl-but-1- ynyl)thieno[3,2-c]pyridin-4-yl]-N-[(3R)-3- piperidyl]-4-(triazolo[4,5-b]pyridin-3- yl)benzamide | 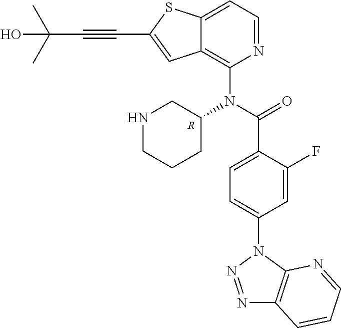 |
| 377 | (R)-4-(3H-[1,2,3]triazolo[4,5-b]pyridin-3- yl)-2-fluoro-N-(2-(4- hydroxyphenyl)thieno[3,2-c]pyridin-4-yl)- N-(piperidin-3-yl)benzamide | 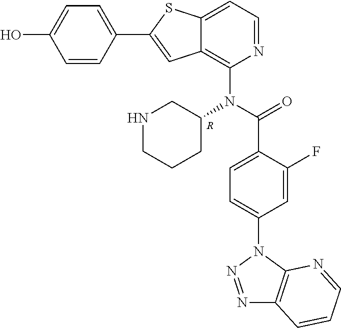 |
| 378 | 2-fluoro-N-[2-(4- methoxyphenyl)thieno[3,2-c]pyridin-4-yl]- N-[(3R)-3-piperidyl]-4-(triazolo[4,5- b]pyridin-3-yl)benzamide | 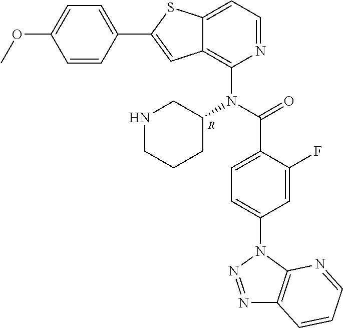 |
| 379 | 4-[4-[[2-fluoro-4-(triazolo[4,5-b]pyridin-3- yl)benzoyl]-[(3R)-3-piperidyl]amino] thieno[3,2-c]pyridin-2-yl]benzoic acid | 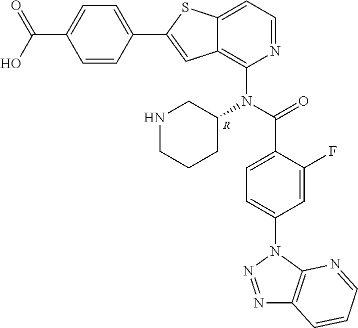 |
| 380 | 2-fluoro-N-[2-[4- (methylcarbamoyl)phenyl]thieno[3,2- c]pyridin-4-yl]-N-[(3R)-3-piperidyl]-4- (triazolo[4,5-b]pyridin-3-yl)benzamide | 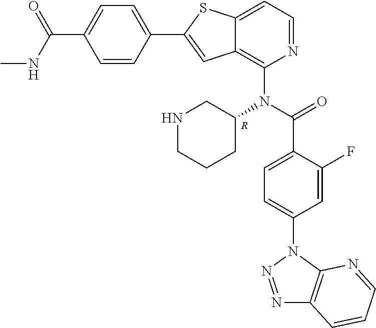 |
| 381 | 2-fluoro-N-[2-(3- hydroxyphenyl)thieno[3,2-c]pyridin-4-yl]- N-[(3R)-3-piperidyl]-4-(triazolo[4,5- b]pyridin-3-yl)benzamide | 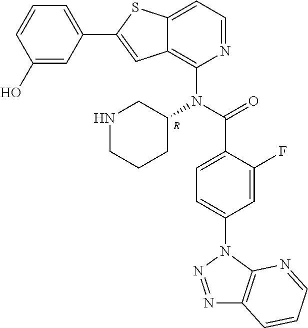 |
| 382 | N-(2-ethynylthieno[3,2-c]pyridin-4-yl)-2- fluoro-N-[(3R)-3-piperidyl]-4- (triazolo[4,5-b]pyridin-3-yl)benzamide | 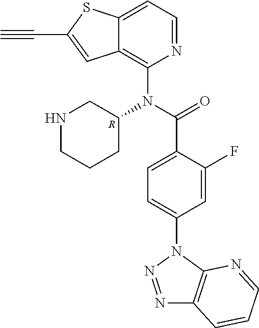 |
| 383 | N-[2-(2-amino-4-pyridyl)thieno[3,2- c]pyridin-4-yl]-2-fluoro-N-[(3R)-3- piperidyl]-4-(triazolo[4,5-b]pyridin-3- yl)benzamide | 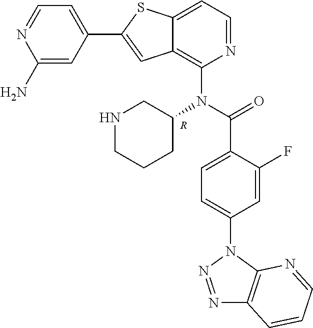 |
| 384 | 2-fluoro-N-[(3R)-3-piperidyl]-N-[2-(4- pyridyl)thieno[2,3-c]pyridin-7-yl]-4- (triazolo[4,5-b]pyridin-3-yl)benzamide | 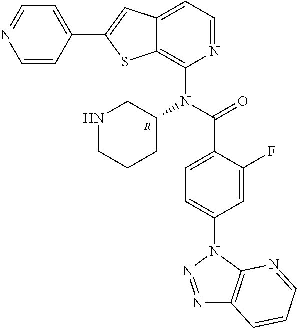 |
| 385 | (R)-4-(3H-[1,2,3]triazolo[4,5-b]pyridin-3- yl)-2-fluoro-N-(2-(3- methoxyphenyl)thieno[3,2-c]pyridin-4-yl)- N-(piperidin-3-yl)benzamide | 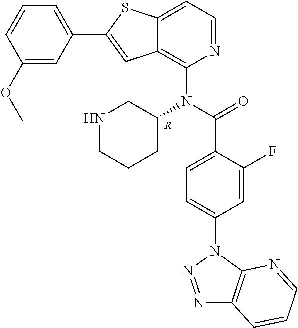 |
| 386 | N-[2-(2-cyano-4-pyridyl)thieno[3,2- c]pyridin-4-yl]-2-fluoro-N-[(3R)-3- piperidyl]-4-(triazolo[4,5-b]pyridin-3- yl)benzamide | 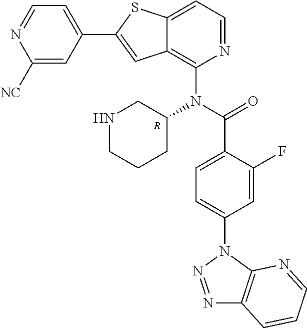 |
| 387 | N-[2-(4-cyanophenyl)thieno[3,2-c]pyridin- 4-yl]-2-fluoro-N-[(3R)-3-piperidyl]-4- (triazolo[4,5-b]pyridin-3-yl)benzamide | 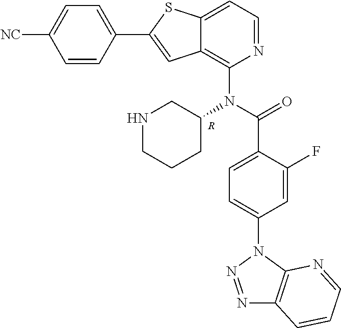 |
| 388 | 4-(5-amino-1-methyl-pyrazol-4-yl)-2- fluoro-N-(8-methyl-1-isoquinolyl)-N- [(3R)-3-piperidyl]benzamide | 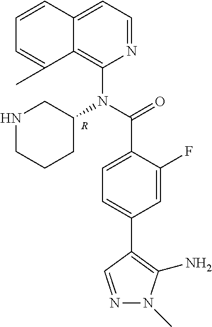 |
| 389 | 4-(5-amino-2-methyl-1H-imidazol-4-yl)-2- fluoro-N-(8-methyl-1-isoquinolyl)-N- [(3R)-3-piperidyl]benzamide | 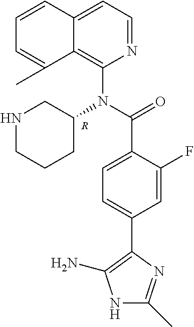 |
| 390 | 4-(5-bromo-2-methyl-triazol-4-yl)-2- fluoro-N-(8-methyl-1-isoquinolyl)-N- [(3R)-3-piperidyl] benzamide | 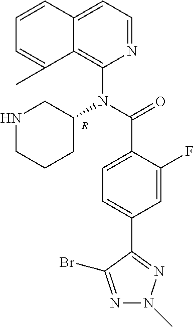 |
| 391 | 4-(3-amino-1-methyl-pyrazol-4-yl)-2- fluoro-N-(8-methyl-1-isoquinolyl)-N- [(3R)-3-piperidyl] benzamide | 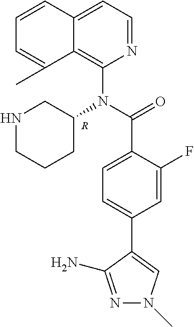 |
| 392 | 4-(4-amino-2,5-dimethyl-pyrazol-3-yl)-2- fluoro-N-(8-methyl-1-isoquinolyl)-N- [(3R)-3-piperidyl]benzamide | 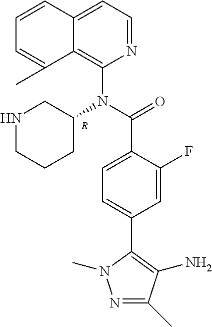 |
| 393 | (R)-N-(8-chloroisoquinolin-1-yl)-2-fluoro- 4-(4-methyl-1H-1,2,3-triazol-1-yl)-N- (piperidin-3-yl)benzamide | 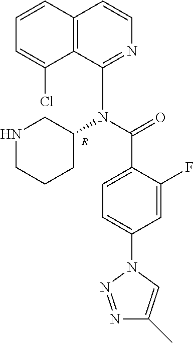 |
| 394 | (R)-2-fluoro-4-(5-methyl-1,3,4-oxadiazol- 2-yl)-N-(8-methylisoquinolin-1-yl)-N- (piperidin-3-yl)benzamide | 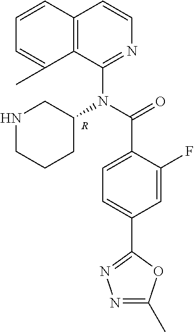 |
| 395 | (R)-N-(6-(1H-pyrazol-4-yl)isoquinolin-1- yl)-4-(2-methyl-2H-tetrazol-5-yl)-N- (piperidin-3-yl)benzamide | 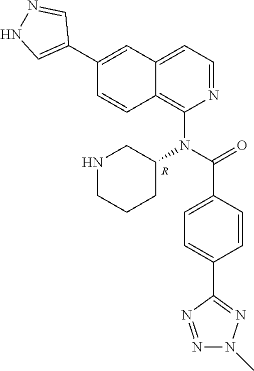 |
| 396 | (R)-4-(3H-[1,2,3]triazolo[4,5-b]pyridin-3- yl)-N-(6-ethynylisoquinolin-1-yl)-2-fluoro- N-(piperidin-3-yl)benzamide | 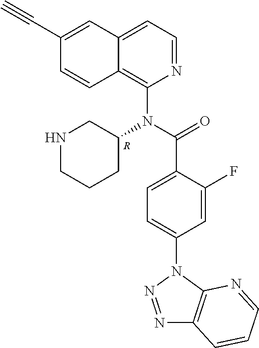 |
| 397 | (pyridin-3-yl)isoquinolin-1-yl)-4- (pyrimidin-2-ylamino)benzamide | 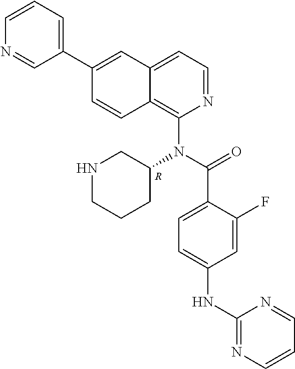 |
| 398 | (R)-4-(3H-[1,2,3]triazolo[4,5-b]pyridin-3- yl)-2-fluoro-N-(6-(3-hydroxyprop-1-yn-1- yl)isoquinolin-1-yl)-N-(piperidin-3- yl)benzamide | 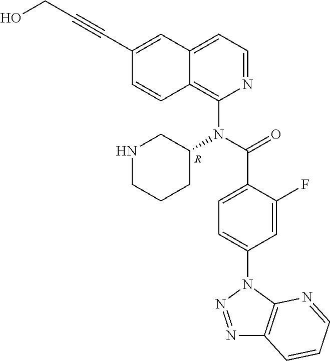 |
| 399 | 3-acrylamido-N-(8-methylisoquinolin-1- yl)-N-((R)-piperidin-3-yl)piperidine-1- carboxamide | 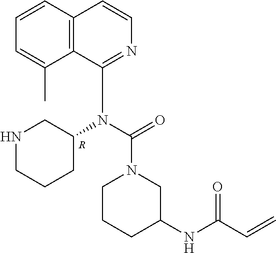 |
| 400 | methyl (R)-2-((3-fluoro-4-((8- methylisoquinolin-1-yl)(piperidin-3- yl)carbamoyl)phenyl)amino)pyrimidine-4- carboxylate | 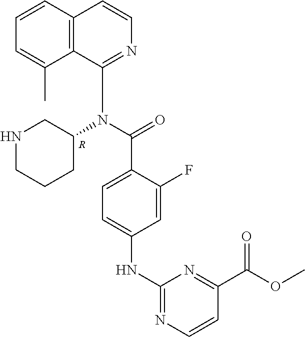 |
| 401 | (R)-2-((3-fluoro-4-((8-methylisoquinolin- 1-yl)(piperidin-3- yl)carbamoyl)phenyl)amino)pyrimidine-4- carboxylic acid | 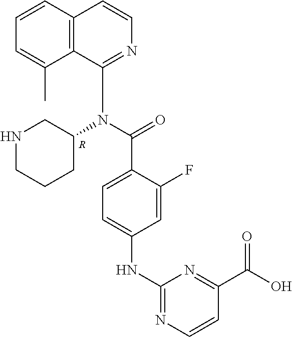 |
| 402 | (R)-2-((3-fluoro-4-((8-methylisoquinolin- 1-yl)(piperidin-3- yl)carbamoyl)phenyl)amino)pyrimidine-4- carboxamide | 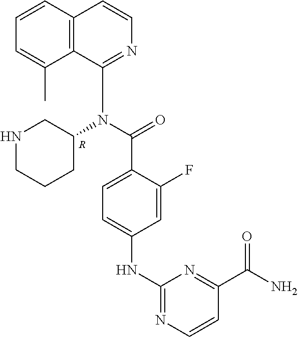 |
| 403 | 4-(1-methyltriazol-4-yl)-N-[(3R)-3- piperidyl]-N-[8-[2-(3-pyridyl)ethyl]-1- isoquinolyl]benzamide | 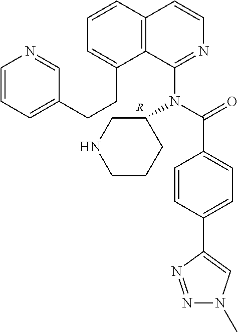 |
| 405 | (R)-N-(6-(1H-tetrazol-5-yl)isoquinolin-1- yl)-2-fluoro-4-(1-methyl-1H-1,2,3-triazol- 4-yl)-N-(piperidin-3-yl)benzamide | 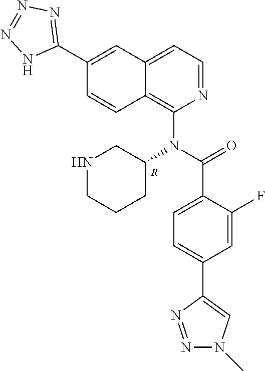 |
| 406 | (R,E)-3-(1-(4-(3H-[1,2,3]triazolo[4,5- b]pyridin-3-yl)-2-fluoro-N-(piperidin-3- yl)benzamido)isoquinolin-6-yl)acrylic acid | 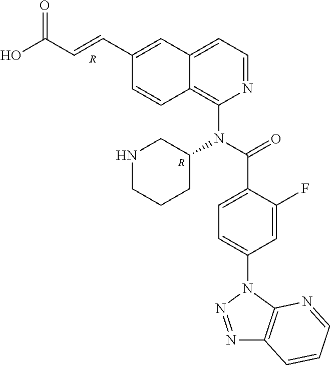 |
| 407 | ((R)-3-(1-(4-(3H-[1,2,3] triazolo[4,5- b]pyridin-3-yl)-2-fluoro-N-(piperidin-3-yl) benzamido) isoquinolin-6-yl)propanoic acid | 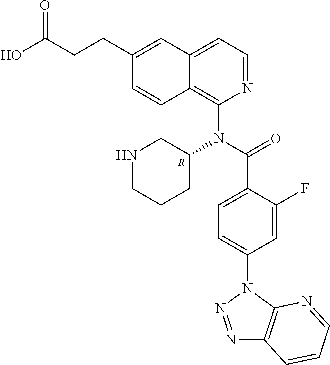 |
| 408 | (R)-2-fluoro-N-(8-methylisoquinolin-1-yl)- 4-((4-morpholinopyrimidin-2-yl)amino)-N- (piperidin-3-yl)benzamide | 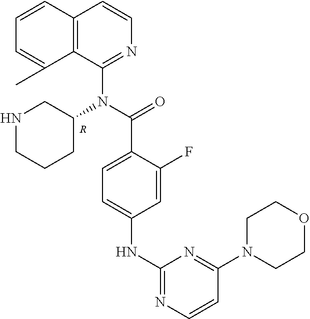 |
| 409 | (R)-2-fluoro-4-((5-fluoropyrimidin-2- yl)amino)-N-(8-methylisoquinolin-1-yl)-N- (piperidin-3-yl)benzamide | 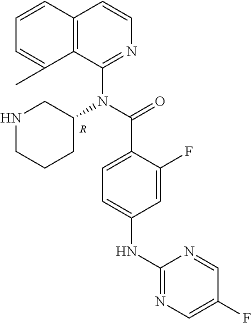 |
| 410 | (R)-4-((5-aminopyrimidin-2-yl)amino)-2- fluoro-N-(8-methylisoquinolin-1-yl)-N- (piperidin-3-yl)benzamide | 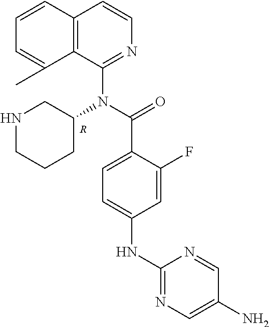 |
| 411 | 2-fluoro-N-[(3R)-3-piperidyl]-N-[2-[2-(4- pyridyl)ethynyl]thieno[3,2-c]pyridin-4-yl]- 4-(triazolo[4,5-b]pyridin-3-yl)benzamide | 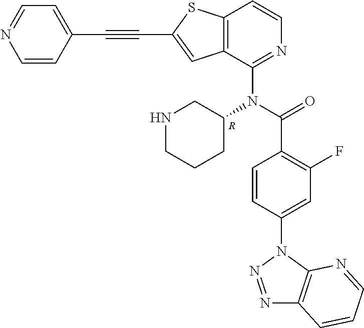 |
| 412 | N-[2-(4-aminophenyl)thieno[3,2-c]pyridin- 4-yl]-2-fluoro-N-[(3R)-3-piperidyl]-4- (triazolo[4,5-b]pyridin-3-yl)benzamide | 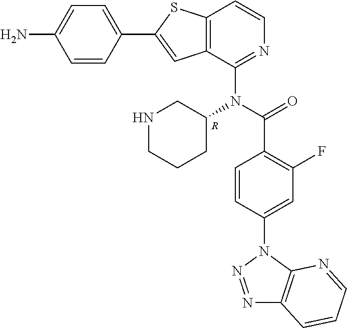 |
| 413 | 2-hydroxyethyl 4-[4-[[2-fluoro-4- (triazolo[4,5-b]pyridin-3-yl)benzoyl]- [(3R)-3-piperidyl]amino]thieno[3,2- c]pyridin-2-yl]benzoate | 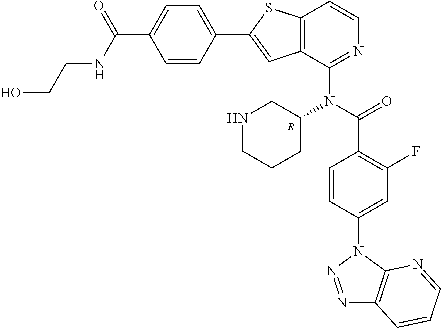 |
| 414 | 2-fluoro-N-[(3R)-3-piperidyl]-N-[2-(4- sulfamoylphenyl)thieno[3,2-c]pyridin-4- yl]-4-(triazolo[4,5-b]pyridin-3- yl)benzamide | 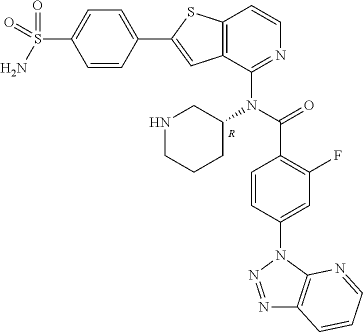 |
| 415 | 2-fluoro-N-[2-(1-isobutyltriazol-4- yl)thieno[3,2-c]pyridin-4-yl]-N-[(3R)-3- piperidyl]-4-(triazolo[4,5-b]pyridin-3- yl)benzamide | 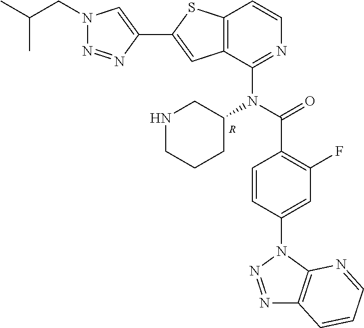 |
| 416 | 2-fluoro-N-[(3R)-3-piperidyl]-N-(2-thiazol- 4-ylthieno[3,2-c]pyridin-4-yl)-4- (triazolo[4,5-b]pyridin-3-yl)benzamide | 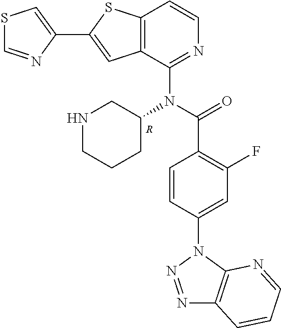 |
| 417 | 2-fluoro-N-[(3R)-3-piperidyl]-N-(2-thiazol- 2-ylthieno[3,2-c]pyridin-4-yl)-4- (triazolo[4,5-b]pyridin-3-yl)benzamide | 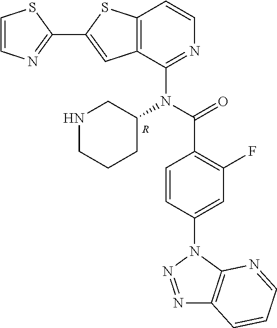 |
| 418 | (R)-4-(3H-[1,2,3]triazolo[4,5-b]pyridin-3- yl)-2-fluoro-N-(2-(1-(2-hydroxyethyl)-1H- pyrazol-3-yl)thieno[3,2-c]pyridin-4-yl)-N- (piperidin-3-yl)benzamide | 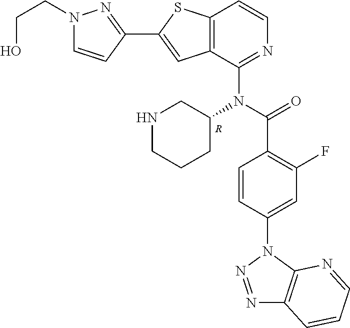 |
| 419 | 2-fluoro-N-[2-(3- methylsulfonylphenyl)thieno[3,2- c]pyridin-4-yl]-N-[(3R)-3-piperidyl]-4- (triazolo[4,5-b]pyridin-3-yl)benzamide | 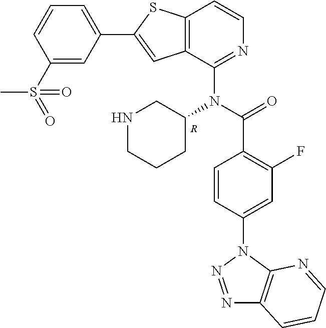 |
| 420 | N-[2-(3-amino-1,2-benzoxazol-5- yl)thieno[3,2-c]pyridin-4-yl]-2-fluoro-N- [(3R)-3-piperidyl]-4-(triazolo[4,5- b]pyridin-3-yl)benzamide | 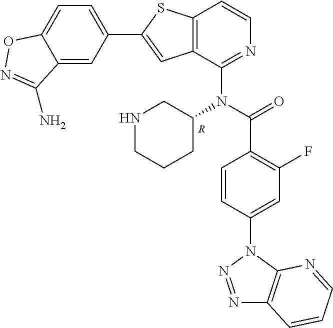 |
| 421 | (R)-4-(3H-[1,2,3]triazolo[4,5-b]pyridin-3- yl)-2-fluoro-N-(8-methyl-6-(pyridin-3- yl)isoquinolin-1-yl)-N-(piperidin-3- yl)benzamide | 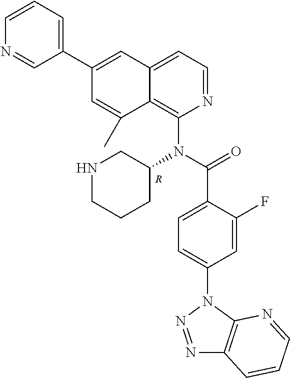 |
| 422 | (R)-4-(3H-[1,2,3]triazolo[4,5-b]pyridin-3- yl)-2-fluoro-N-(8-methyl-6-(1H-pyrazol-4- yl)isoquinolin-1-yl)-N-(piperidin-3- yl)benzamide | 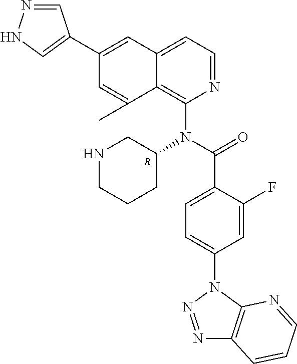 |
| 423 | 2-fluoro-N-[2-(1-isopropylpyrazol-4-yl) thieno [3,2-c]pyridin-4-yl]-N-[(3R)-3- piperidyl]-4-(triazolo[4,5-b]pyridin-3- yl)benzamide | 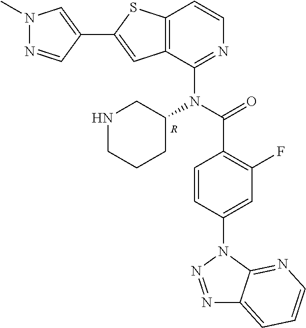 |
| 424 | 2-fluoro-N-[2-[3- (hydroxymethyl)phenyl]thieno[3,2- c]pyridin-4-yl]-N-[(3R)-3-piperidyl]-4- (triazolo[4,5-b]pyridin-3-yl)benzamide | 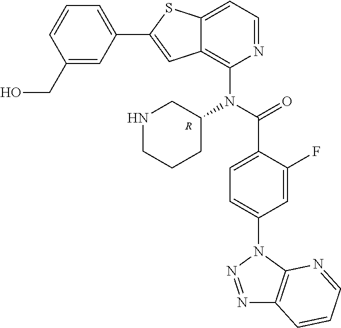 |
| 425 | N-[2-(5-cyano-2-thienyl) thieno[3,2- c]pyridin-4-yl]-2-fluoro-N-[(3R)-3- piperidyl]-4-(triazolo[4,5-b]pyridin-3- yl)benzamide | 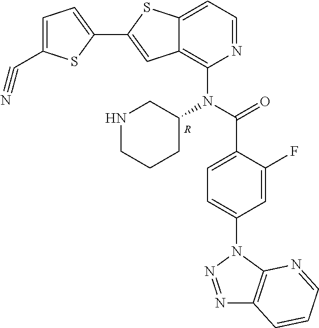 |
| 426 | 2-fluoro-N-[2-[5-(hydroxymethyl)-2- thienyl] thieno [3, 2-c] pyridin-4-yl]-N- [(3R)-3-piperidyl]-4-(triazolo [4, 5-b] pyridin-3-yl) benzamide | 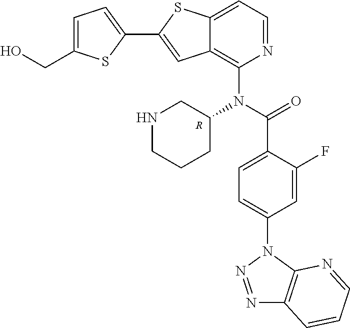 |
| 427 | 2-fluoro-N-[2-[2-(hydroxymethyl)thiazol- 4-yl]thieno[3,2-c]pyridin-4-yl]-N-[(3R)-3- piperidyl]-4-(triazolo[4,5-b]pyridin-3- yl)benzamide | 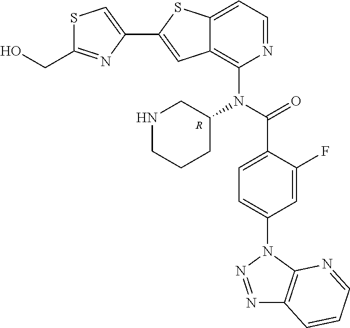 |
| 428 | 2-fluoro-N-[2-(2- hydroxyphenyl)thieno[3,2-c]pyridin-4-yl]- N-[(3R)-3-piperidyl]-4-(triazolo[4,5- b]pyridin-3-yl)benzamide | 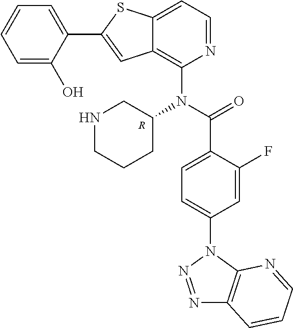 |
| 429 | methyl 4-[4-[[2-fluoro-4-(triazolo[4,5- b]pyridin-3-yl)benzoyl]-[(3R)-3- piperidyl]amino]thieno[3,2-c]pyridin-2- yl]thiazole-2-carboxylate | 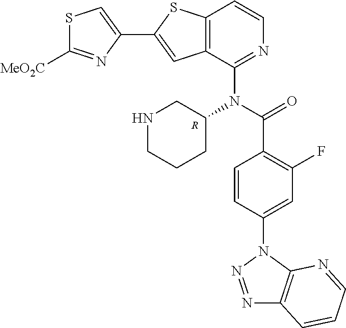 |
| 430 | 2-fluoro-N-[4-[(E)-2-(1-methylpyrazol-4- yl)vinyl]-2-pyridyl]-N-[(3R)-3-piperidyl]- 4-(triazolo[4,5-b]pyridin-3-yl)benzamide | 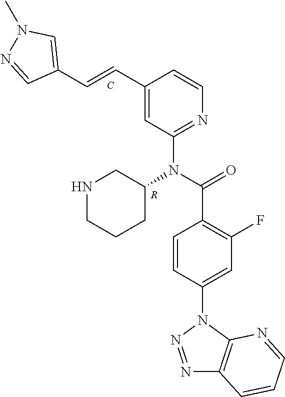 |
| 431 | 2-fluoro-N-[(3R)-3-piperidyl]-N-[4-[(E)-2- (3-pyridyl)vinyl]-2-pyridyl]-4- (triazolo[4,5-b]pyridin-3-yl)benzamide | 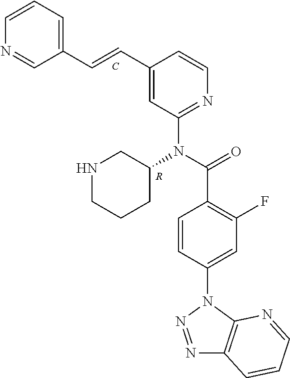 |
| 432 | (R)-2-fluoro-N-(8-methylisoquinolin-1-yl)- N-(piperidin-3-yl)-4-(5,6,7,8-tetrahydro- [1,2,4]triazolo[4,3-a]pyridin-3- yl)benzamide | 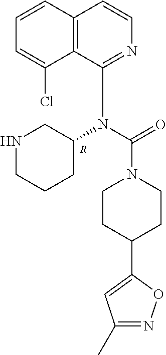 |
| 433 | ((R)-2-fluoro-N-(piperidin-3-yl)-N-(2- (pyridin-4-yl)thieno[3,2-c]pyridin-4-yl)-4- (pyrimidin-2-ylamino)benzamide | 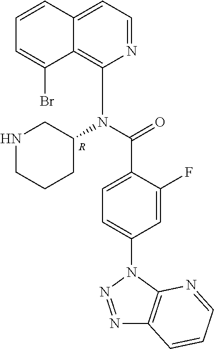 |
| 434 | (R)-4-(3H-[1,2,3]triazolo[4,5-b]pyridin-3- yl)-N-(2-([1,2,4]triazolo[1,5-a]pyridin-6- yl)thieno[3,2-c]pyridin-4-yl)-2-fluoro-N- (piperidin-3-yl)benzamide | 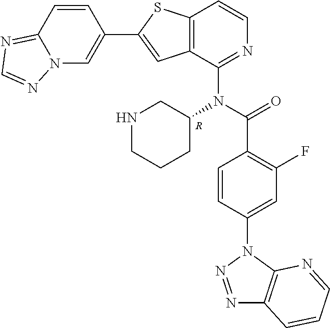 |
| 435 | 2-fluoro-N-[(3R)-3-piperidyl]-N-[2-[1-(2- pyridyl)pyrazol-4-yl]thieno[3,2-c]pyridin- 4-yl]-4-(triazolo[4,5-b]pyridin-3- yl)benzamide | 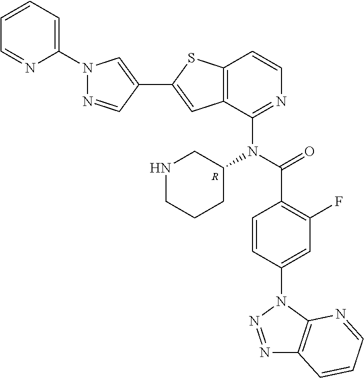 |
| 436 | N-[2-(1-ethylpyrazol-4-yl)thieno[3,2- c]pyridin-4-yl]-2-fluoro-N-[(3R)-3- piperidyl]-4-(triazolo[4,5-b]pyridin-3- yl)benzamide | 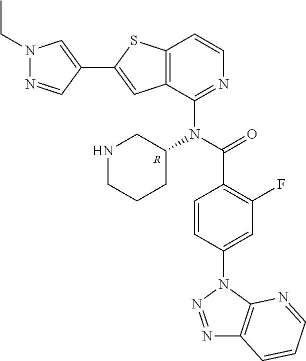 |
| 437 | (R)-4-(3H-[1,2,3]triazolo[4,5-b]pyridin-3- yl)-2-fluoro-N-(2-(1-isopropyl-1H-pyrazol- 4-yl)thieno[3,2-c]pyridin-4-yl)-N- (piperidin-3-yl)benzamide | 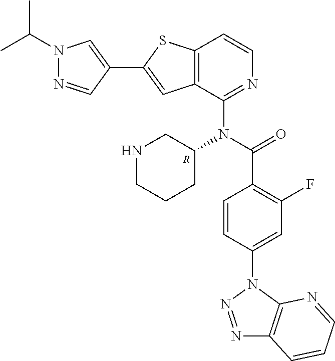 |
| 438 | 2-fluoro-N-[2-(1-methylpyrazol-3- yl)thieno[3,2-c]pyridin-4-yl]-N-[(3R)-3- piperidyl]-4-(triazolo[4,5-b]pyridin-3- yl)benzamide | 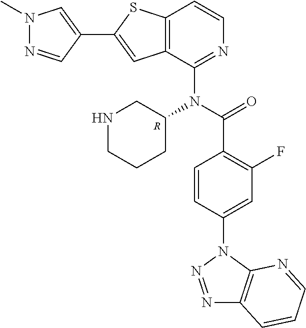 |
| 439 | (R)-4-(3H-[1,2,3]triazolo[4,5-b]pyridin-3- yl)-2-fluoro-N-(6-(1-isopropyl-1H-pyrazol- 5-yl)isoquinolin-1-yl)-N-(piperidin-3- yl)benzamide | 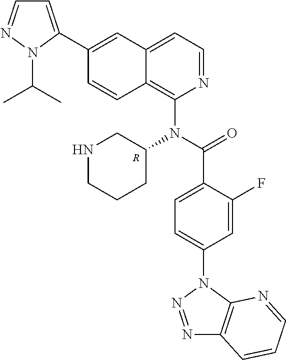 |
| 440 | (R)-4-(3H-[1,2,3]triazolo[4,5-b]pyridin-3- yl)-2-fluoro-N-(6-(2-oxo-1,2- dihydropyridin-4-yl)isoquinolin-1-yl)-N- (piperidin-3-yl)benzamide | 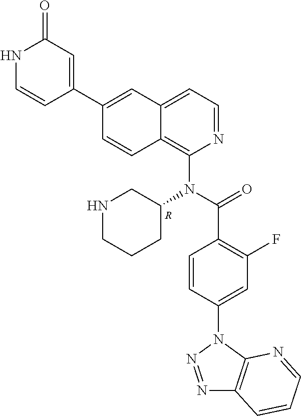 |
| 441 | 2-fluoro-N-(8-methyl-1-isoquinolyl)-N- [(3R, 4S)-4-methyl-3-piperidyl]-4-(1- methyltriazol-4-yl) benzamide | 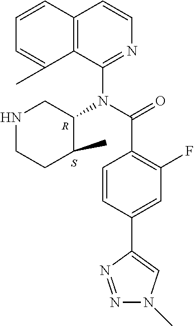 |
| 442 | N-[2-[(E)-3-amino-3-oxo-prop-1- enyl]thieno[3,2-c]pyridin-4-yl]-2-fluoro-N- [(3R)-3-piperidyl]-4-(triazolo[4,5- b]pyridin-3-yl)benzamide | 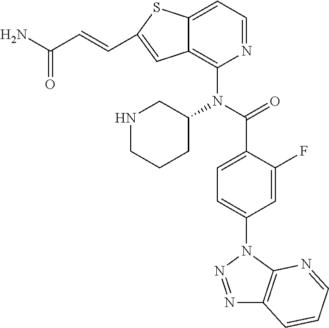 [SMILES: NC(=O)/C=C/c1cc2c(N(C(=O)c3ccc(-n4nnc5cccnc54)cc3F)[C@@H]3CCCNC3)nccc2s1] |
| 443 | 2-fluoro-N-[2-(2-methylthiazol-5- yl)thieno[3,2-c]pyridin-4-yl]-N-[(3R)-3- piperidyl]-4-(triazolo[4,5-b]pyridin-3- yl)benzamide | 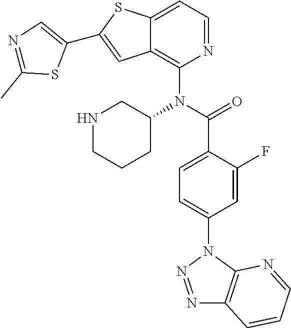 [SMILES: Cc1ncc(-c2cc3c(N(C(=O)c4ccc(-n5nnc6cccnc65)cc4F)[C@@H]4CCCNC4)nccc3s2)s1] |
| 444 | N-(6-chloro-8-methyl-1-isoquinolyl)-2- fluoro-4-(1-methyltriazol-4-yl)-N-[(3R)-3- piperidyl] benzamide | 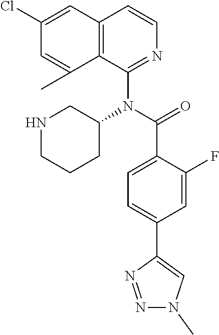 [SMILES: Cc1cc(Cl)cc2ccnc(N(C(=O)c3ccc(-c4cn(C)nn4)cc3F)[C@@H]3CCCNC3)c12] |
| 445 | (R)-2-fluoro-4-(1-methyl-1H-1,2,3-triazol- 4-yl)-N-(6-(1-methyl-1H-pyrazol-4- yl)isoquinolin-1-yl)-N-(piperidin-3- yl)benzamide | 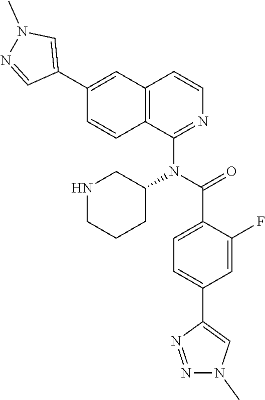 [SMILES: Cn1cc(-c2ccc3c(N(C(=O)c4ccc(-c5cn(C)nn5)cc4F)[C@@H]4CCCNC4)nccc3c2)cn1] |
| 446 | 2-fluoro-N-[(3R)-3-piperidyl]-N-[2-(3- sulfamoylphenyl)thieno[3,2-c]pyridin-4- yl]-4-(triazolo[4,5-b]pyridin-3- yl)benzamide | 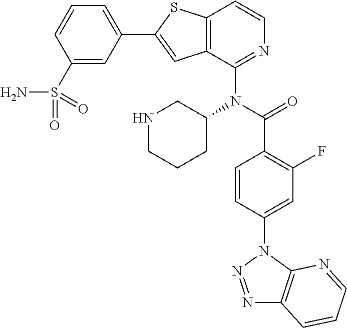 [SMILES: NS(=O)(=O)c1cccc(-c2cc3c(N(C(=O)c4ccc(-n5nnc6cccnc65)cc4F)[C@@H]4CCCNC4)nccc3s2)c1] |
| 447 | N-[2-[3-(2-amino-2-oxo- ethyl)phenyl]thieno[3,2-c]pyridin-4-yl]-2- fluoro-N-[(3R)-3-piperidyl]-4- (triazolo[4,5-b] pyridin-3-yl)benzamide | 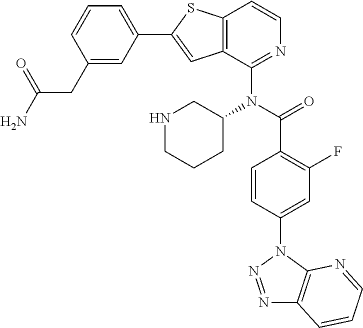 [SMILES: NC(=O)Cc1cccc(-c2cc3c(N(C(=O)c4ccc(-n5nnc6cccnc65)cc4F)[C@@H]4CCCNC4)nccc3s2)c1] |
| 448 | N-[2-(4-carbamoylphenyl)thieno[3,2- c]pyridin-4-yl]-2-fluoro-N-[(3R)-3- piperidyl]-4-(triazolo[4,5-b] pyridin-3- yl)benzamide | 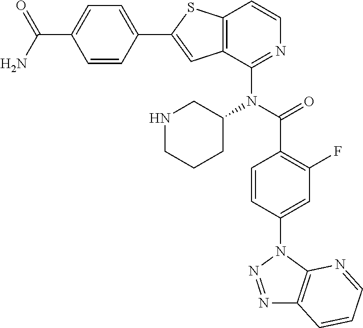 [SMILES: NC(=O)c1ccc(-c2cc3c(N(C(=O)c4ccc(-n5nnc6cccnc65)cc4F)[C@@H]4CCCNC4)nccc3s2)cc1] |
| 449 | 2-fluoro-N-[2-(3-oxazol-2-ylphenyl) thieno [3, 2-c] pyridin-4-yl]-N-[(3R)-3-piperidyl]- 4-(triazolo [4, 5-b] pyridin-3-yl) benzamide | 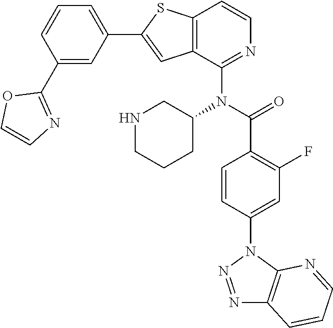 [SMILES: O=C(c1ccc(-n2nnc3cccnc32)cc1F)N(c1nccc2sc(-c3cccc(-c4ncco4)c3)cc12)[C@@H]1CCCNC1] |
| 450 | 2-fluoro-N-[(3R)-3-piperidyl]-N-[2- ([1,2,4]triazolo[1,5-a]pyridin-2- yl)thieno[3,2-c]pyridin-4-yl]-4- (triazolo[4,5-b]pyridin-3-yl)benzamide | 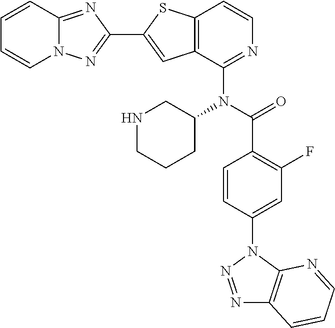 [SMILES: O=C(c1ccc(-n2nnc3cccnc32)cc1F)N(c1nccc2sc(-c3nc4ccccn4n3)cc12)[C@@H]1CCCNC1] |
| 451 | 2-fluoro-N-[(3R)-3-piperidyl]-N-(2- pyrazin-2-ylthieno[3,2-c]pyridin-4-yl)-4- (triazolo[4,5-b]pyridin-3-yl)benzamide | 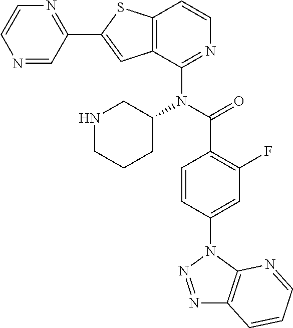 [SMILES: O=C(c1ccc(-n2nnc3cccnc32)cc1F)N(c1nccc2sc(-c3cnccn3)cc12)[C@@H]1CCCNC1] |
| 452 | N-[2-(3-carbamoylphenyl)thieno[3,2- c]pyridin-4-yl]-2-fluoro-N-[(3R)-3- piperidyl]-4-(triazolo[4,5-b]pyridin-3- yl)benzamide | 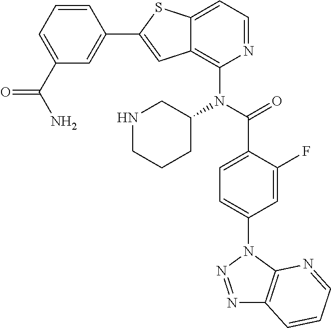 [SMILES: NC(=O)c1cccc(-c2cc3c(N(C(=O)c4ccc(-n5nnc6cccnc65)cc4F)[C@@H]4CCCNC4)nccc3s2)c1] |
| 453 | (R)-1-(4-(3H-[1,2,3]triazolo[4,5-b]pyridin- 3-yl)-2-fluoro-N-(piperidin-3- yl)benzamido)isoquinoline-6-carboxamide | 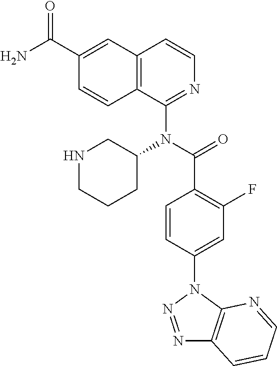 [SMILES: NC(=O)c1ccc2c(N(C(=O)c3ccc(-n4nnc5cccnc54)cc3F)[C@@H]3CCCNC3)nccc2c1] |
| 454 | 2-fluoro-N-[(3R)-3-piperidyl]-4- (triazolo[4,5-b]pyridin-3-yl)-N-[2-[6- (trifluoromethyl)-3-pyridyl]thieno[3,2- c]pyridin-4-yl]benzamide | 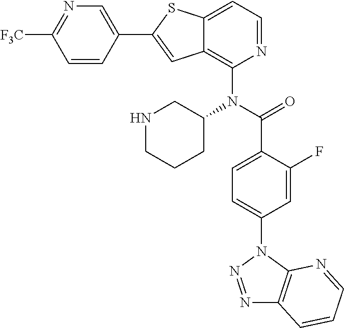 [SMILES: O=C(c1ccc(-n2nnc3cccnc32)cc1F)N(c1nccc2sc(-c3ccc(C(F)(F)F)nc3)cc12)[C@@H]1CCCNC1] |
| 455 | (R)-4-(3H-[1,2,3]triazolo[4,5-b]pyridin-3- yl)-2-fluoro-N-(piperidin-3-yl)-N-(2- (pyridazin-4-yl)thieno[3,2-c]pyridin-4- yl)benzamide | 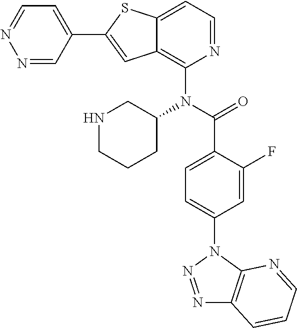 [SMILES: O=C(c1ccc(-n2nnc3cccnc32)cc1F)N(c1nccc2sc(-c3ccnnc3)cc12)[C@@H]1CCCNC1] |
| 456 | (R)-4-(3H-[1,2,3]triazolo[4,5-b]pyridin-3- yl)-2-fluoro-N-(piperidin-3-yl)-N-(2- (pyridin-2-yl)thieno[3,2-c]pyridin-4- yl)benzamide | 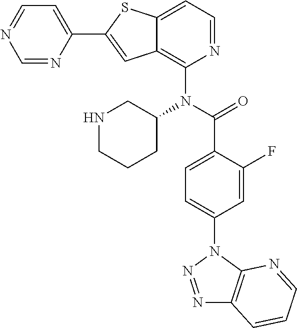 [SMILES: O=C(c1ccc(-n2nnc3cccnc32)cc1F)N(c1nccc2sc(-c3ccncn3)cc12)[C@@H]1CCCNC1] |
| 457 | N-[2-(2-aminopyrimidin-4-yl)thieno[3,2- c]pyridin-4-yl]-2-fluoro-N-[(3R)-3- piperidyl]-4-(triazolo[4,5-b]pyridin-3- yl)benzamide | 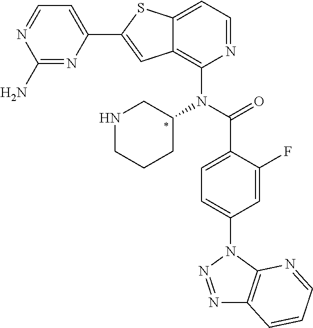 [SMILES: Nc1nccc(-c2cc3c(N(C(=O)c4ccc(-n5nnc6cccnc65)cc4F)[C@@H]4CCCNC4)nccc3s2)n1] |
| 458 | N-[2-[2-(dimethylamino)pyrimidin-4- yl]thieno[3,2-c]pyridin-4-yl]-2-fluoro-N- [(3R)-3-piperidyl]-4-(triazolo[4,5- b]pyridin-3-yl)benzamide | 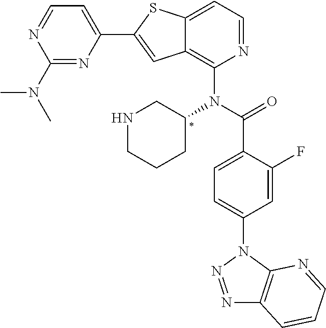 [SMILES: CN(C)c1nccc(-c2cc3c(N(C(=O)c4ccc(-n5nnc6cccnc65)cc4F)[C@@H]4CCCNC4)nccc3s2)n1] |
| 459 | 2-fluoro-N-[2-(2-oxo-1H-pyridin-4- yl)thieno[3,2-c]pyridin-4-yl]-N-[(3R)-3- piperidyl]-4-(triazolo[4,5-b] pyridin-3- yl)benzamide | 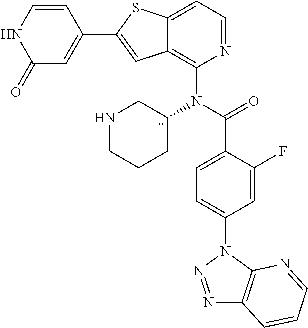 [SMILES: O=C(c1ccc(-n2nnc3cccnc32)cc1F)N(c1nccc2sc(-c3cc[nH]c(=O)c3)cc12)[C@@H]1CCCNC1] |
| 460 | 2-fluoro-N-[2-(2-methoxy-4- pyridyl)thieno[3,2-c]pyridin-4-yl]-N-[(3R)- 3-piperidyl]-4-(triazolo[4,5-b]pyridin-3- yl)benzamide | 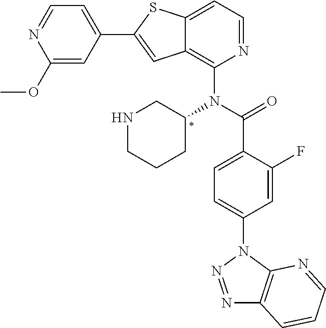 [SMILES: COc1cc(-c2cc3c(N(C(=O)c4ccc(-n5nnc6cccnc65)cc4F)[C@@H]4CCCNC4)nccc3s2)ccn1] |
| 461 | N-[2-(2-ethyl-4-pyridyl)thieno[3,2- c]pyridin-4-yl]-2-fluoro-N-[(3R)-3- piperidyl]-4-(triazolo[4,5-b]pyridin-3- yl)benzamide | 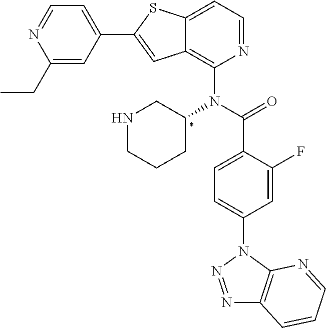 [SMILES: CCc1cc(-c2cc3c(N(C(=O)c4ccc(-n5nnc6cccnc65)cc4F)[C@@H]4CCCNC4)nccc3s2)ccn1] |
| 462 | 2-fluoro-N-[(3R)-3-piperidyl]-4- (triazolo[4,5-b]pyridin-3-yl)-N-[2-[2- (trifluoromethyl)-4-pyridyl]thieno[3,2- c]pyridin-4-yl]benzamide | 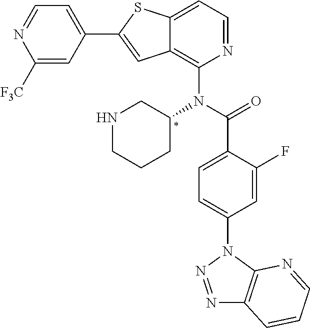 [SMILES: O=C(c1ccc(-n2nnc3cccnc32)cc1F)N(c1nccc2sc(-c3ccnc(C(F)(F)F)c3)cc12)[C@@H]1CCCNC1] |
| 463 | (R)-4-(3H-[1,2,3]triazolo[4,5-b]pyridin-3- yl)-N-(2-(2-(2-cyanopropan-2-yl)pyridin- 4-yl)thieno[3,2-c]pyridin-4-yl)-2-fluoro-N- (piperidin-3-yl)benzamide | 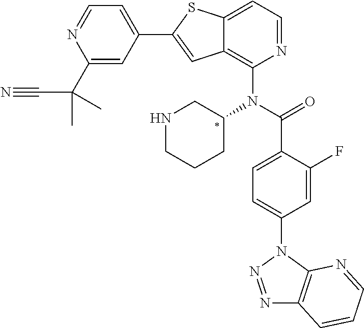 [SMILES: CC(C)(C#N)c1cc(-c2cc3c(N(C(=O)c4ccc(-n5nnc6cccnc65)cc4F)[C@@H]4CCCNC4)nccc3s2)ccn1] |
| 464 | 2-fluoro-N-(2-phenylthieno[3,2-c]pyridin- 4-yl)-N-[(3R)-3-piperidyl]-4-(triazolo[4,5- b]pyridin-3-yl)benzamide | 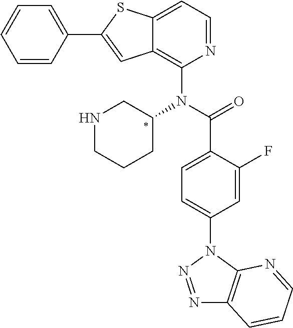 [SMILES: O=C(c1ccc(-n2nnc3cccnc32)cc1F)N(c1nccc2sc(-c3ccccc3)cc12)[C@@H]1CCCNC1] |
| 465 | 2-fluoro-N-[(3R)-3-piperidyl]-N-[2-(3- pyridylmethyl)thieno[3,2-c]pyridin-4-yl]- 4-(triazolo[4,5-b]pyridin-3-yl)benzamide | 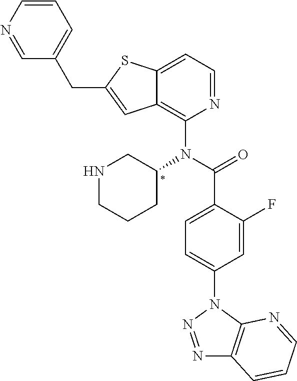 [SMILES: O=C(c1ccc(-n2nnc3cccnc32)cc1F)N(c1nccc2sc(Cc3cccnc3)cc12)[C@@H]1CCCNC1] |
| 466 | N-(2-benzylthieno[3,2-c]pyridin-4-yl)-2- fluoro-N-[(3R)-3-piperidyl]-4-(triazolo[4,5- b]pyridin-3-yl)benzamide | 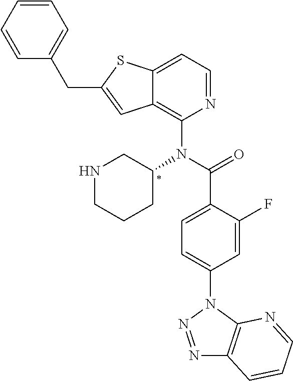 [SMILES: O=C(c1ccc(-n2nnc3cccnc32)cc1F)N(c1nccc2sc(Cc3ccccc3)cc12)[C@@H]1CCCNC1] |
| 467 | 2-fluoro-N-[2-(3-formylphenyl)thieno[3,2- c]pyridin-4-yl]-N-[(3R)-3-piperidyl]-4- (triazolo[4,5-b]pyridin-3-yl)benzamide | 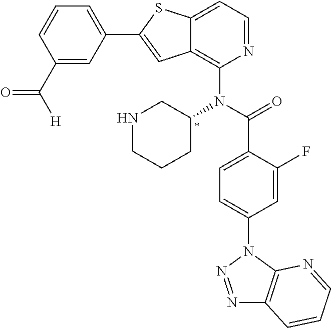 [SMILES: O=Cc1cccc(-c2cc3c(N(C(=O)c4ccc(-n5nnc6cccnc65)cc4F)[C@@H]4CCCNC4)nccc3s2)c1] |
| 468 | (R)-2-fluoro-N-(3-methylthieno[3,2- c]pyridin-4-yl)-N-(piperidin-3-yl)-4- (pyrimidin-2-ylamino)benzamide | 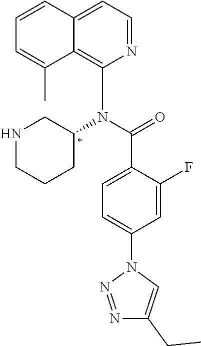 [SMILES: CCc1cn(-c2ccc(C(=O)N(c3nccc4cccc(C)c34)[C@@H]3CCCNC3)c(F)c2)nn1] |
| 469 | (R)-2-fluoro-N-(6-fluoroisoquinolin-1-yl)- 4-(1-methyl-1H-1,2,3-triazol-4-yl)-N- (piperidin-3-yl)benzamide | 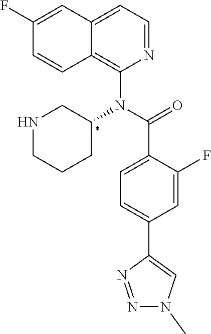 [SMILES: Cn1cc(-c2ccc(C(=O)N(c3nccc4cc(F)ccc34)[C@@H]3CCCNC3)c(F)c2)nn1] |
| 470 | N-(6-chloro-1-isoquinolyl)-2-fluoro-4-(1- methyltriazol-4-yl)-N-[(3R)-3- piperidyl]benzamide | 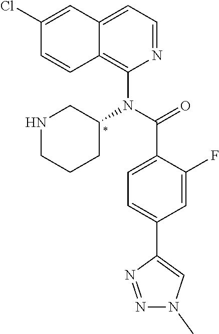 [SMILES: Cn1cc(-c2ccc(C(=O)N(c3nccc4cc(Cl)ccc34)[C@@H]3CCCNC3)c(F)c2)nn1] |
| 471 | (R)-2-fluoro-N-(6-methoxyisoquinolin-1- yl)-4-(1-methyl-1H-1,2,3-triazol-4-yl)-N- (piperidin-3-yl)benzamide | 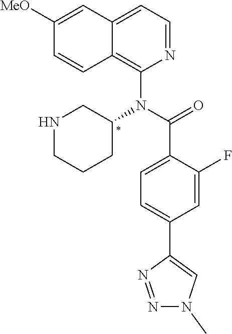 [SMILES: COc1ccc2c(N(C(=O)c3ccc(-c4cn(C)nn4)cc3F)[C@@H]3CCCNC3)nccc2c1] |
| 472 | (R)-N-(6-cyanoisoquinolin-1-yl)-2-fluoro- 4-(1-methyl-1H-1,2,3-triazol-4-yl)-N- (piperidin-3-yl)benzamide | 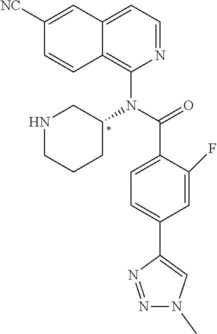 [SMILES: Cn1cc(-c2ccc(C(=O)N(c3nccc4cc(C#N)ccc34)[C@@H]3CCCNC3)c(F)c2)nn1] |
| 473 | (R)-4-((1,3,5-triazin-2-yl)amino)-2-fluoro- N-(8-methylisoquinolin-1-yl)-N-(piperidin- 3-yl)benzamide | 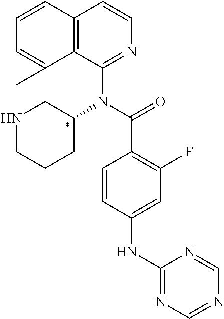 [SMILES: Cc1cccc2ccnc(N(C(=O)c3ccc(Nc4ncncn4)cc3F)[C@@H]3CCCNC3)c12] |
| 474 | (R)-2-fluoro-N-(6-fluoro-8- methylisoquinolin-1-yl)-4-(1-methyl-1H- 1,2,3-triazol-4-yl)-N-(piperidin-3- yl)benzamide | 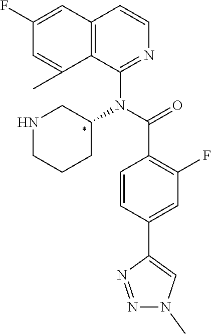 [SMILES: Cc1cc(F)cc2ccnc(N(C(=O)c3ccc(-c4cn(C)nn4)cc3F)[C@@H]3CCCNC3)c12] |
| 475 | N-(2-bromothieno [3, 2-c] pyridin-4-yl)-3- hydroxy-4-(1-methylpyrazol-4-yl)-N- [(3R)-3-piperidyl] benzamide | 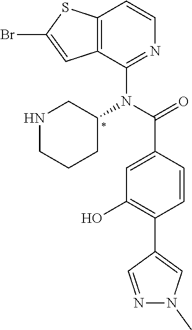 [SMILES: Cn1cc(-c2ccc(C(=O)N(c3nccc4sc(Br)cc34)[C@@H]3CCCNC3)cc2O)cn1] |
| 476 | 3-hydroxy-N-[2-(5-hydroxypent-1-ynyl) thieno [3, 2-c] pyridin-4-yl]-4-(1- methylpyrazol-4-yl)-N-[(3R)-3-piperidyl] benzamide | 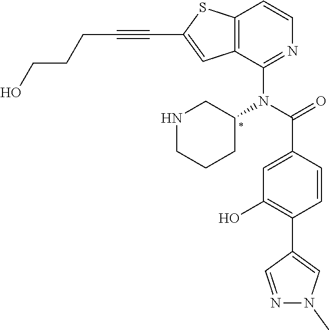 [SMILES: Cn1cc(-c2ccc(C(=O)N(c3nccc4sc(C#CCCCO)cc34)[C@@H]3CCCNC3)cc2O)cn1] |
| 477 | N-(2-cyanothieno [3, 2-c] pyridin-4-yl)-2- fluoro-4-(1-methyltriazol-4-yl)-N-[(3R)-3- piperidyl] benzamide | 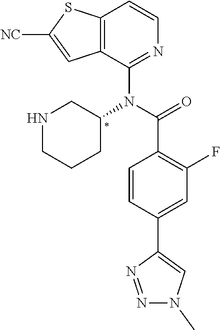 [SMILES: Cn1cc(-c2ccc(C(=O)N(c3nccc4sc(C#N)cc34)[C@@H]3CCCNC3)c(F)c2)nn1] |
| 478 | (R)-N-(2-(2-aminopyridin-4-yl)thieno[3,2- c]pyridin-4-yl)-2-fluoro-4-(1-methyl-1H- 1,2,3-triazol-4-yl)-N-(piperidin-3- yl)benzamide | 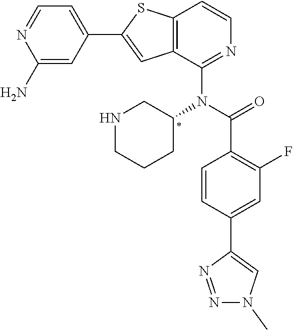 [SMILES: Cn1cc(-c2ccc(C(=O)N(c3nccc4sc(-c5ccnc(N)c5)cc34)[C@@H]3CCCNC3)c(F)c2)nn1] |
| 479 | (R)-2-fluoro-N-(2-(3-hydroxyprop-1-yn-1- yl)thieno[3,2-c]pyridin-4-yl)-4-(1-methyl- 1H-1,2,3-triazol-4-yl)-N-(piperidin-3- yl)benzamide | 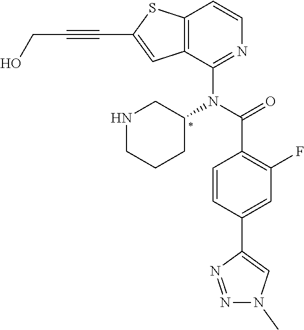 [SMILES: Cn1cc(-c2ccc(C(=O)N(c3nccc4sc(C#CCO)cc34)[C@@H]3CCCNC3)c(F)c2)nn1] |
| 480 | N-[2-(3-carbamoylphenyl) thieno [3, 2-c] pyridin-4-yl]-2-fluoro-4-(1-methyltriazol- 4-yl)-N-[(3R)-3-piperidyl] benzamide | 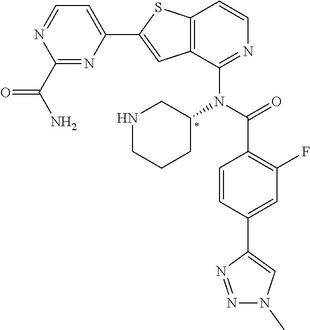 [SMILES: Cn1cc(-c2ccc(C(=O)N(c3nccc4sc(-c5ccnc(C(N)=O)n5)cc34)[C@@H]3CCCNC3)c(F)c2)nn1] |
| 481 | (R)-2-fluoro-N-(2-(5- (hydroxymethyl)thiophen-2-yl)thieno[3,2- c]pyridin-4-yl)-4-(1-methyl-1H-1,2,3- triazol-4-yl)-N-(piperidin-3-yl)benzamide | 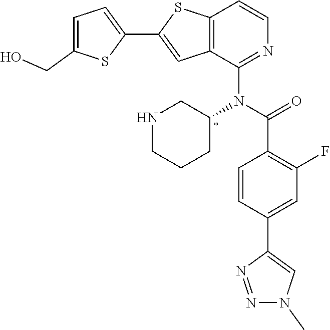 [SMILES: Cn1cc(-c2ccc(C(=O)N(c3nccc4sc(-c5ccc(CO)s5)cc34)[C@@H]3CCCNC3)c(F)c2)nn1] |
| 482 | 2-fluoro-N-[(3R)-3-piperidyl]-N-(2- pyrazin-2-ylthieno[3,2-c]pyridin-4-yl)-4- (triazolo [4,5-b]pyridin-3-yl)benzamide | 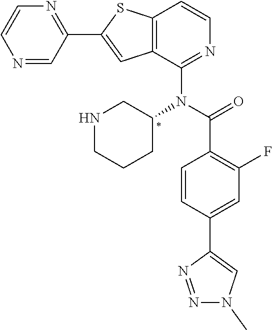 [SMILES: Cn1cc(-c2ccc(C(=O)N(c3nccc4sc(-c5cnccn5)cc34)[C@@H]3CCCNC3)c(F)c2)nn1] |
| 483 | (R)-2-fluoro-4-(1-methyl-1H-1,2,3-triazol- 4-yl)-N-(2-(2-methylthiazol-5- yl)thieno[3,2-c]pyridin-4-yl)-N-(piperidin- 3-yl)benzamide | 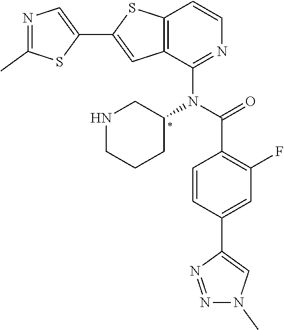 [SMILES: Cc1ncc(-c2cc3c(N(C(=O)c4ccc(-c5cn(C)nn5)cc4F)[C@@H]4CCCNC4)nccc3s2)s1] |
| 484 | N-[2-(2-aminopyrimidin-4-yl)thieno[3,2- c]pyridin-4-yl]-2-fluoro-4-(1- methyltriazol-4-yl)-N-[(3R)-3- piperidyl]benzamide | 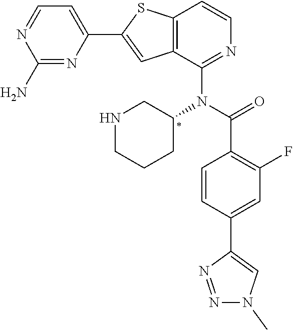 [SMILES: Cn1cc(-c2ccc(C(=O)N(c3nccc4sc(-c5ccnc(N)n5)cc34)[C@@H]3CCCNC3)c(F)c2)nn1] |
| 485 | 2-fluoro-4-(1-methyltriazol-4-yl)-N-[(3R)- 3-piperidyl]-N-[2-[1-(3-pyridylmethyl) pyrazol-4-yl] thieno [3, 2-c] pyridin-4-yl] benzamide | 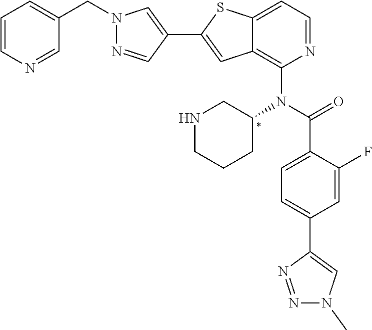 [SMILES: Cn1cc(-c2ccc(C(=O)N(c3nccc4sc(-c5cnn(Cc6cccnc6)c5)cc34)[C@@H]3CCCNC3)c(F)c2)nn1] |
| 486 | N-[2-[(E)-3-amino-3-oxo-prop-1- enyl]thieno[3,2-c]pyridin-4-yl]-2-fluoro-4- (1-methyltriazol-4-yl)-N-[(3R)-3- piperidyl]benzamide | 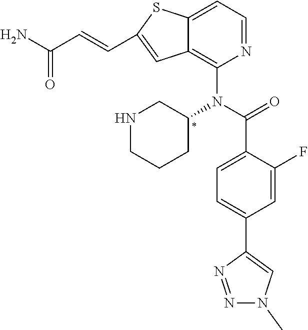 [SMILES: Cn1cc(-c2ccc(C(=O)N(c3nccc4sc(/C=C/C(N)=O)cc34)[C@@H]3CCCNC3)c(F)c2)nn1] |
| 487 | N-(2-benzylthieno[3,2-c]pyridin-4-yl)-2- fluoro-4-(1-methyltriazol-4-yl)-N-[(3R)-3- piperidyl]benzamide | 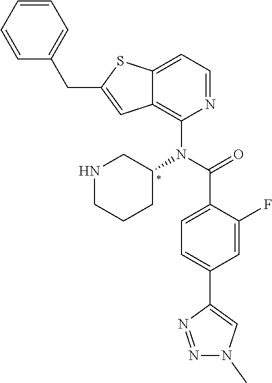 [SMILES: Cn1cc(-c2ccc(C(=O)N(c3nccc4sc(Cc5ccccc5)cc34)[C@@H]3CCCNC3)c(F)c2)nn1] |
| 488 | 2-fluoro-4-(1-methyltriazol-4-yl)-N-[(3R)- 3-piperidyl]-N-[2-(3- pyridylmethyl)thieno[3,2-c]pyridin-4- yl]benzamide | 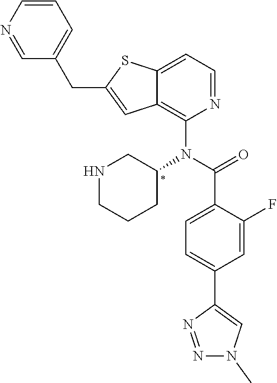 [SMILES: Cn1cc(-c2ccc(C(=O)N(c3nccc4sc(Cc5cccnc5)cc34)[C@@H]3CCCNC3)c(F)c2)nn1] |
| 489 | N-[2-[1-(2-amino-2-oxo-ethyl)pyrazol-4- yl]thieno[3,2-c]pyridin-4-yl]-2-fluoro-4-(1- methyltriazol-4-yl)-N-[(3R)-3- piperidyl]benzamide | 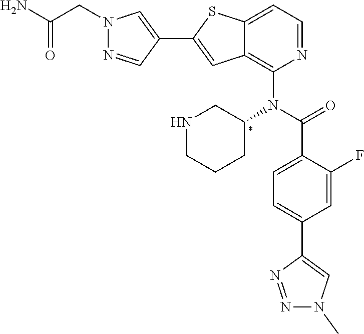 [SMILES: Cn1cc(-c2ccc(C(=O)N(c3nccc4sc(-c5cnn(CC(N)=O)c5)cc34)[C@@H]3CCCNC3)c(F)c2)nn1] |
| 490 | (R)-2-fluoro-N-(6-(3- (hydroxymethyl)phenyl)isoquinolin-1-yl)- 4-(1-methyl-1H-1,2,3-triazol-4-yl)-N- (piperidin-3-yl)benzamide | 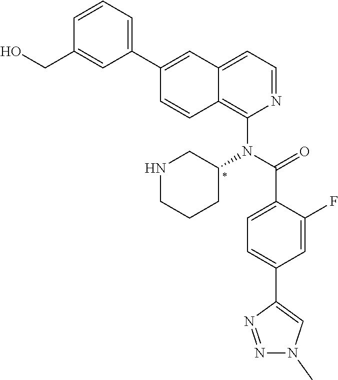 [SMILES: Cn1cc(-c2ccc(C(=O)N(c3nccc4cc(-c5cccc(CO)c5)ccc34)[C@@H]3CCCNC3)c(F)c2)nn1] |
| 491 | (R)-N-(6-(2-aminopyridin-4- yl)isoquinolin-1-yl)-2-fluoro-4-(1-methyl- 1H-1,2,3-triazol-4-yl)-N-(piperidin-3- yl)benzamide | 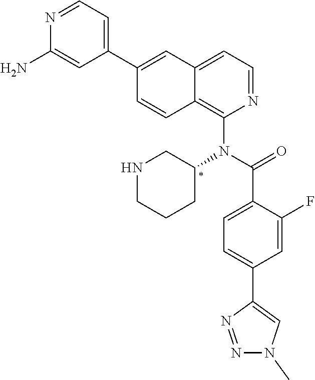 [SMILES: Cn1cc(-c2ccc(C(=O)N(c3nccc4cc(-c5ccnc(N)c5)ccc34)[C@@H]3CCCNC3)c(F)c2)nn1] |
| 492 | (R)-2-fluoro-N-(6-(1-(2-hydroxyethyl)-1H- pyrazol-4-yl)isoquinolin-1-yl)-4-(1- methyl-1H-1,2,3-triazol-4-yl)-N- (piperidin-3-yl)benzamide | 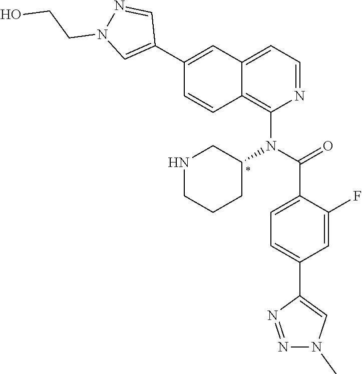 [SMILES: Cn1cc(-c2ccc(C(=O)N(c3nccc4cc(-c5cnn(CCO)c5)ccc34)[C@@H]3CCCNC3)c(F)c2)nn1] |
| 493 | (R)-2-fluoro-N-(6-(2- hydroxyphenyl)isoquinolin-1-yl)-4-(1- methyl-1H-1,2,3-triazol-4-yl)-N- (piperidin-3-yl)benzamide | 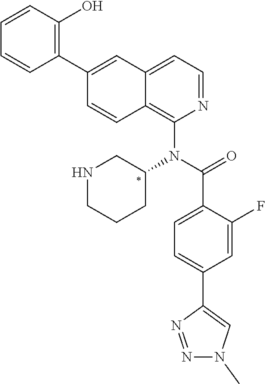 [SMILES: Cn1cc(-c2ccc(C(=O)N(c3nccc4cc(-c5ccccc5O)ccc34)[C@@H]3CCCNC3)c(F)c2)nn1] |
| 494 | (R,E)-3-(1-(2-fluoro-4-(1-methyl-1H-1,2,3- triazol-4-yl)-N-(piperidin-3- yl)benzamido)isoquinolin-6-yl)acrylic acid | 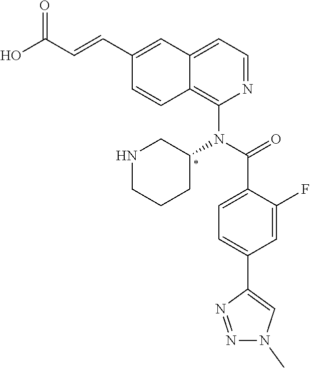 [SMILES: Cn1cc(-c2ccc(C(=O)N(c3nccc4cc(/C=C/C(=O)O)ccc34)[C@@H]3CCCNC3)c(F)c2)nn1] |
| 495 | (R)-2-fluoro-N-(6-(3- hydroxyphenyl)isoquinolin-1-yl)-4-(1- methyl-1H-1,2,3-triazol-4-yl)-N- (piperidin-3-yl)benzamide | 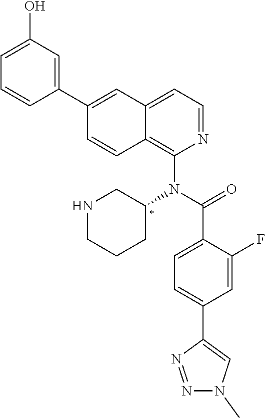 [SMILES: Cn1cc(-c2ccc(C(=O)N(c3nccc4cc(-c5cccc(O)c5)ccc34)[C@@H]3CCCNC3)c(F)c2)nn1] |
| 496 | (R,E)-N-(6-(3-amino-3-oxoprop-1-en-1- yl)isoquinolin-1-yl)-2-fluoro-4-(1-methyl- 1H-1,2,3-triazol-4-yl)-N-(piperidin-3- yl)benzamide | 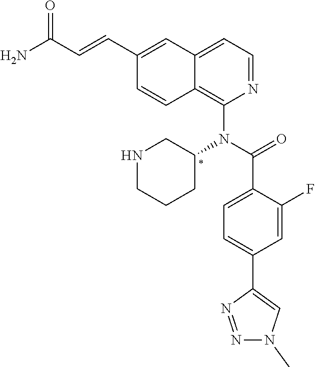 [SMILES: Cn1cc(-c2ccc(C(=O)N(c3nccc4cc(/C=C/C(N)=O)ccc34)[C@@H]3CCCNC3)c(F)c2)nn1] |
| 497 | N-(8-methylisoquinolin-1-yl)-3-(3-oxo-3- (pyridin-4-ylamino)propyl)-N-((R)- piperidin-3-yl)piperidine-1-carboxamide | 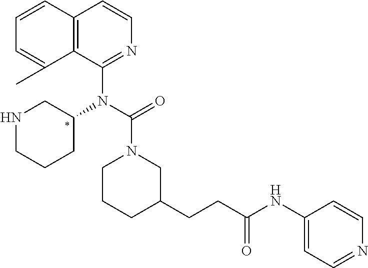 [SMILES: Cc1cccc2ccnc(N(C(=O)N3CCCC(CCC(=O)Nc4ccncc4)C3)[C@@H]3CCCNC3)c12] |
| 498 | 3-(3-((1H-indazol-5-yl)amino)-3- oxopropyl)-N-(8-methylisoquinolin-1-yl)- N-((R)-piperidin-3-yl)piperidine-1- carboxamide | 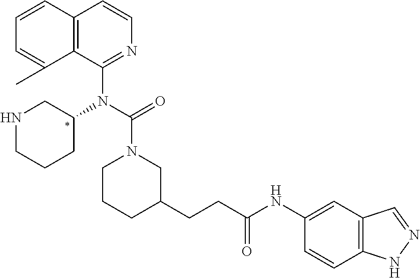 [SMILES: Cc1cccc2ccnc(N(C(=O)N3CCCC(CCC(=O)Nc4ccc5[nH]ncc5c4)C3)[C@@H]3CCCNC3)c12] |
| 499 | 3-(3-((1H-indazol-6-yl)amino)-3- oxopropyl)-N-(8-methylisoquinolin-1-yl)- N-((R)-piperidin-3-yl)piperidine-1- carboxamide | 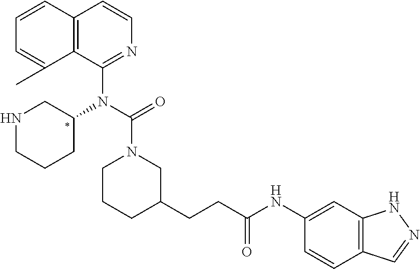 [SMILES: Cc1cccc2ccnc(N(C(=O)N3CCCC(CCC(=O)Nc4ccc5cn[nH]c5c4)C3)[C@@H]3CCCNC3)c12] |
| 500 | (E)-3-[4-[[2-fluoro-4-(triazolo[4,5- b]pyridin-3-yl)benzoyl]-[(3R)-3- piperidyl]amino]thieno[3,2-c]pyridin-2- yllprop-2-enoic acid | 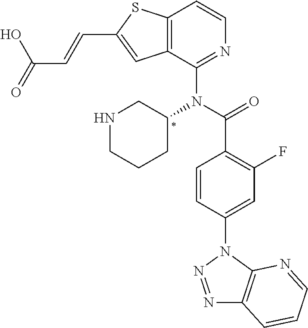 [SMILES: O=C(O)/C=C/c1cc2c(N(C(=O)c3ccc(-n4nnc5cccnc54)cc3F)[C@@H]3CCCNC3)nccc2s1] |
| 501 | ethyl (E)-3-[4-[[2-fluoro-4-(triazolo[4,5- b]pyridin-3-yl)benzoyl]-[(3R)-3- piperidyl]amino]thieno[3,2-c]pyridin-2- yl]prop-2-enoate | 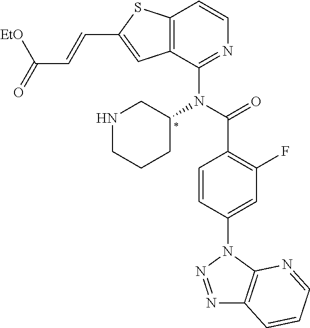 [SMILES: CCOC(=O)/C=C/c1cc2c(N(C(=O)c3ccc(-n4nnc5cccnc54)cc3F)[C@@H]3CCCNC3)nccc2s1] |
| 502 | 4-[4-[[2-fluoro-4-(triazolo[4,5-b]pyridin-3- yl)benzoyl]-[(3R)-3- piperidyl]amino]thieno[3,2-c]pyridin-2- yl]thiazole-2-carboxylic acid | 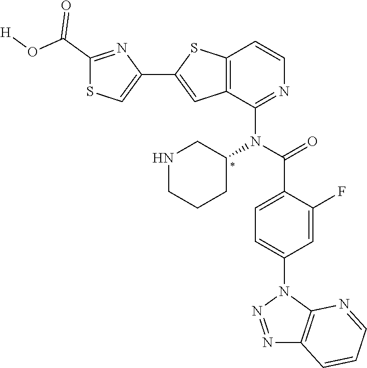 [SMILES: O=C(O)c1nc(-c2cc3c(N(C(=O)c4ccc(-n5nnc6cccnc65)cc4F)[C@@H]4CCCNC4)nccc3s2)cs1] |
| 503 | 2-fluoro-N-[(3R)-3-piperidyl]-N-[2-[2-(4- pyridyl)ethyl]thieno[3,2-c]pyridin-4-yl]-4- (triazolo[4,5-b]pyridin-3-yl)benzamide | 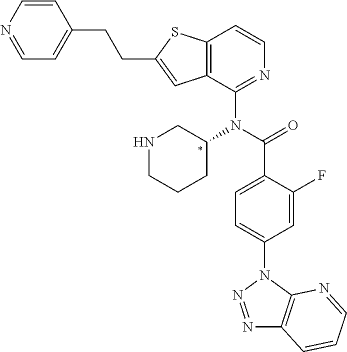 [SMILES: O=C(c1ccc(-n2nnc3cccnc32)cc1F)N(c1nccc2sc(CCc3ccncc3)cc12)[C@@H]1CCCNC1] |
| 504 | (R,Z)-2-fluoro-N-(2-(3-hydroxyprop-1-en- 1-yl)thieno[3,2-c]pyridin-4-yl)-4-(1- methyl-1H-1,2,3-triazol-4-yl)-N- (piperidin-3-yl)benzamide | 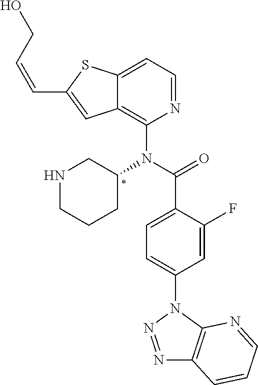 [SMILES: O=C(c1ccc(-n2nnc3cccnc32)cc1F)N(c1nccc2sc(/C=C\CO)cc12)[C@@H]1CCCNC1] |
| 505 | 2-fluoro-4-(1-methyltriazol-4-yl)-N-[(3R)- 3-piperidyl]-N-[2-(4-pyridyl)thieno[2,3- c]pyridin-7-yl]benzamide | 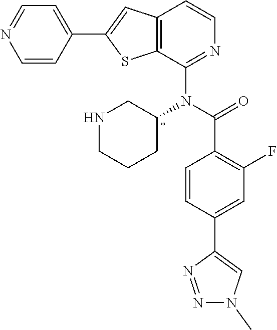 [SMILES: Cn1cc(-c2ccc(C(=O)N(c3nccc4cc(-c5ccncc5)sc34)[C@@H]3CCCNC3)c(F)c2)nn1] |
| 506 | N-(2-bromothieno [3, 2-c] pyridin-4-yl)-2- fluoro-4-(1-methyltriazol-4-yl)-N-[(3R)-3- piperidyl] benzamide | 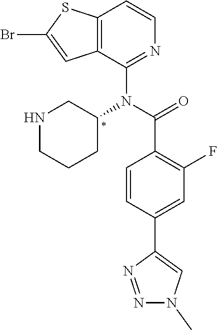 [SMILES: Cn1cc(-c2ccc(C(=O)N(c3nccc4sc(Br)cc34)[C@@H]3CCCNC3)c(F)c2)nn1] |
| 507 | 2-fluoro-4-(1-methyltriazol-4-yl)-N-[(3R)- 3-piperidyl]-N-[2-(2-pyridyl) thieno [3, 2- c] pyridin-4-yl] benzamide | 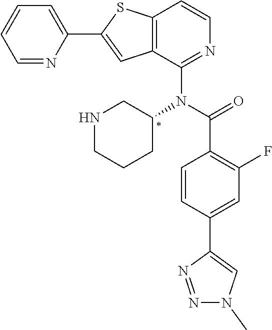 [SMILES: Cn1cc(-c2ccc(C(=O)N(c3nccc4sc(-c5ccccn5)cc34)[C@@H]3CCCNC3)c(F)c2)nn1] |
| 508 | 2-fluoro-4-(1-methyltriazol-4-yl)-N-[(3R)- 3-piperidyl]-N-[2-(3-pyridyl) thieno [3, 2- c] pyridin-4-yl] benzamide | 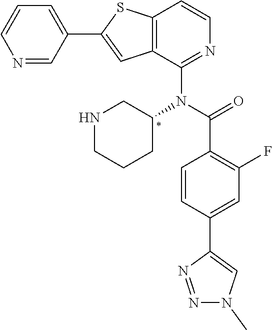 [SMILES: Cn1cc(-c2ccc(C(=O)N(c3nccc4sc(-c5cccnc5)cc34)[C@@H]3CCCNC3)c(F)c2)nn1] |
| 509 | N-[2-(3-aminophenyl)thieno[3,2-c]pyridin- 4-yl]-2-fluoro-4-(1-methyltriazol-4-yl)-N- [(3R)-3-piperidyl]benzamidezamide | 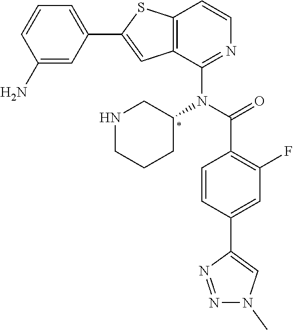 [SMILES: Cn1cc(-c2ccc(C(=O)N(c3nccc4sc(-c5cccc(N)c5)cc34)[C@@H]3CCCNC3)c(F)c2)nn1] |
| 510 | 2-fluoro-4-(1-methyltriazol-4-yl)-N-(2- phenylthieno[3,2-c]pyridin-4-yl)-N-[(3R)- 3-piperidyl]benzamide | 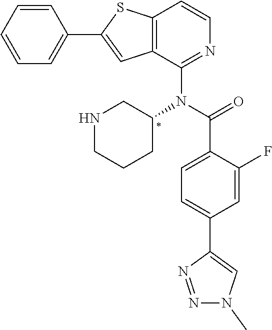 [SMILES: Cn1cc(-c2ccc(C(=O)N(c3nccc4sc(-c5ccccc5)cc34)[C@@H]3CCCNC3)c(F)c2)nn1] |
| 511 | 2-fluoro-4-(1-methyltriazol-4-yl)-N-[(3R)- 3-piperidyl]-N-[2-(3-thienyl)thieno[3,2- c]pyridin-4-yl]benzamide | 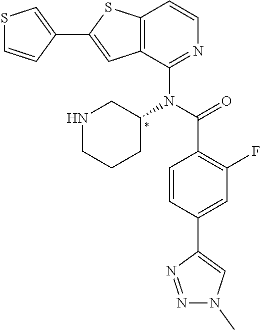 [SMILES: Cn1cc(-c2ccc(C(=O)N(c3nccc4sc(-c5ccsc5)cc34)[C@@H]3CCCNC3)c(F)c2)nn1] |
| 512 | (R)-3-(4-hydroxybut-1-yn-1-yl)-N-(8- methylisoquinolin-1-yl)-N-(piperidin-3- yl)benzamide | 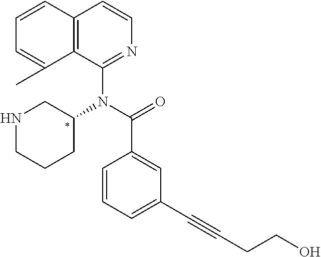 [SMILES: Cc1cccc2ccnc(N(C(=O)c3cccc(C#CCCO)c3)[C@@H]3CCCNC3)c12] |
| 513 | (R)-3-(5-hydroxypent-1-yn-1-yl)-N-(8- methylisoquinolin-1-yl)-N-(piperidin-3- yl)benzamide | 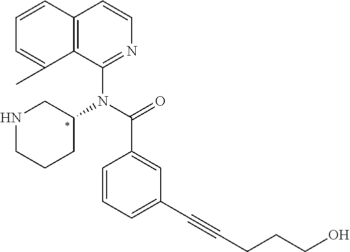 [SMILES: Cc1cccc2ccnc(N(C(=O)c3cccc(C#CCCCO)c3)[C@@H]3CCCNC3)c12] |
| 514 | (R)-3-(6-hydroxyhex-1-yn-1-yl)-N-(8- methylisoquinolin-1-yl)-N-(piperidin-3- yl)benzamide | 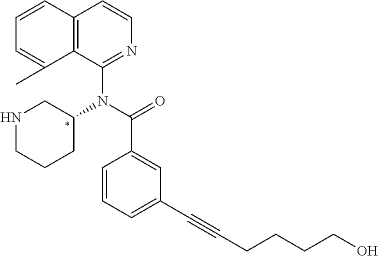 [SMILES: Cc1cccc2ccnc(N(C(=O)c3cccc(C#CCCCCO)c3)[C@@H]3CCCNC3)c12] |
| 515 | (R)-3-(4-aminobut-1-yn-1-yl)-N-(8- methylisoquinolin-1-yl)-N-(piperidin-3- yl)benzamide | 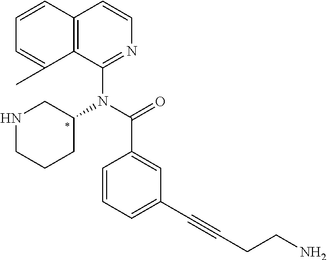 [SMILES: Cc1cccc2ccnc(N(C(=O)c3cccc(C#CCCN)c3)[C@@H]3CCCNC3)c12] |
| 516 | (R)-N-(8-methylisoquinolin-1-yl)-3-(5- oxo-5-(pyridin-4-ylamino)pent-1-yn-1-yl)- N-(piperidin-3-yl)benzamide | 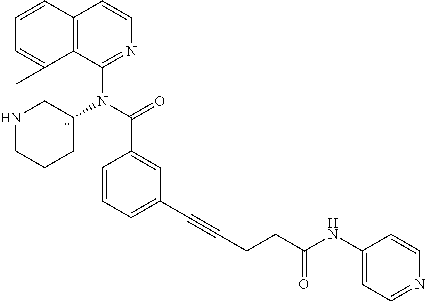 [SMILES: Cc1cccc2ccnc(N(C(=O)c3cccc(C#CCCC(=O)Nc4ccncc4)c3)[C@@H]3CCCNC3)c12] |
| 517 | (R)-N-(8-methylisoquinolin-1-yl)-3-(5- oxo-5-((pyridin-4-ylmethyl)amino)pent-1- yn-1-yl)-N-(piperidin-3-yl)benzamide | 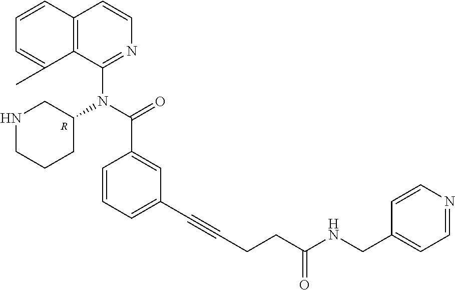 |
| 518 | 3-(5-(3-hydroxypyrrolidin-1-yl)-5- oxopent-1-yn-1-yl)-N-(8- methylisoquinolin-1-yl)-N-((R)-piperidin- 3-yl)benzamide | 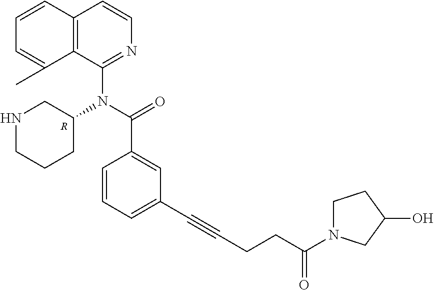 |
| 519 | (R)-N-(8-methylisoquinolin-1-yl)-3-(5- morpholino-5-oxopent-1-yn-1-yl)-N- (piperidin-3-yl)benzamide | 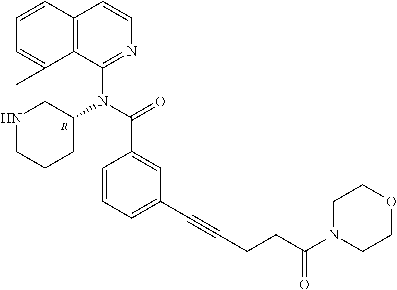 |
| 520 | 3-(5-((1-hydroxypropan-2-yl)amino)-5- oxopent-1-yn-1-yl)-N-(8- methylisoquinolin-1-yl)-N-((R)-piperidin- 3-yl)benzamide | 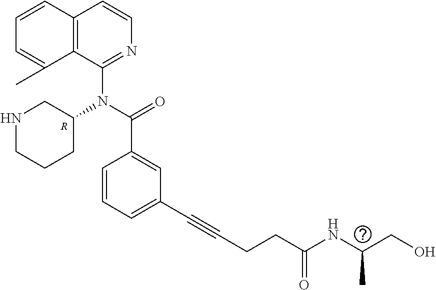 |
| 521 | (R)-2-fluoro-4-(1-methyl-1H-1,2,3-triazol- 4-yl)-N-(2-(4- (methylcarbamoyl)phenyl)thieno[3,2- c]pyridin-4-yl)-N-(piperidin-3- yl)benzamide | 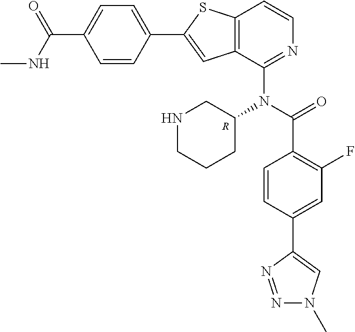 |
| 522 | 2-fluoro-N-[2-[4- (isopropylcarbamoyl)phenyl]thieno[3,2- c]pyridin-4-yl]-4-(1-methyltriazol-4-yl)-N- [(3R)-3-piperidyl]benzamide | 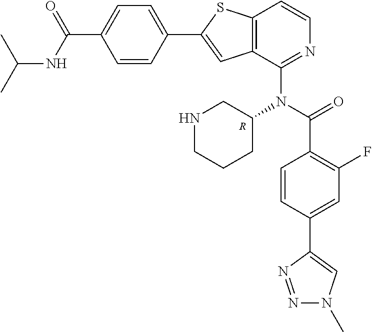 |
| 523 | 2-fluoro-4-(1-methyltriazol-4-yl)-N-[2-[4- (phenylcarbamoyl)phenyl]thieno[3,2- c]pyridin-4-yl]-N-[(3R)-3- piperidyl]benzamide | 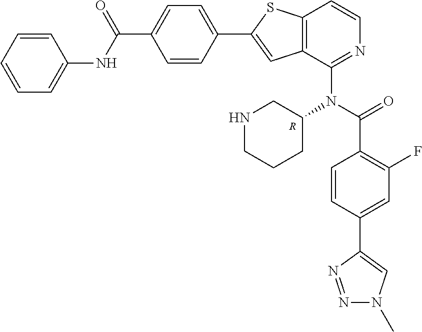 |
| 524 | N-[2-(3-aminophenyl)thieno[3,2-c]pyridin- 4-yl]-2-fluoro-4-(1-methyltriazol-4-yl)-N- [(3R)-3-piperidyl]benzamidezamide | 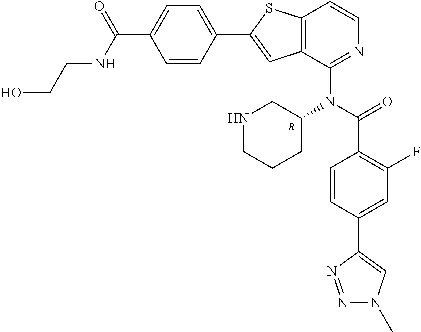 |
| 525 | 2-fluoro-N-[2-[(E)-3-(methylamino)-3- oxo-prop-1-enyl]thieno[3,2-c]pyridin-4- yl]-N-[(3R)-3-piperidyl]-4-(triazolo[4,5- b]pyridin-3-yl)benzamide | 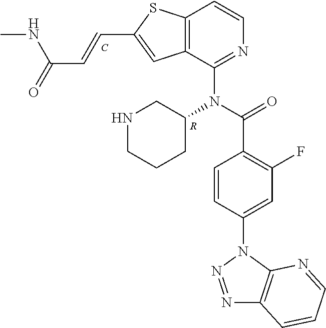 |
| 526 | N-[2-[(E)-3-(dimethylamino)-3-oxo-prop- 1-enyl]thieno[3,2-c]pyridin-4-yl]-2-fluoro- N-[(3R)-3-piperidyl]-4-(triazolo[4,5- b]pyridin-3-yl)benzamide | 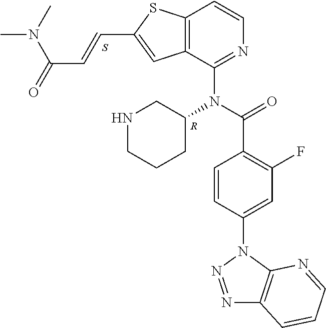 |
| 527 | 4-[4-[[2-fluoro-4-(triazolo[4,5-b]pyridin-3- yl)benzoyl]-[(3R)-3- piperidyl]amino]thieno[3,2-c]pyridin-2-yl]- N-methyl-thiazole-2-carboxamide | 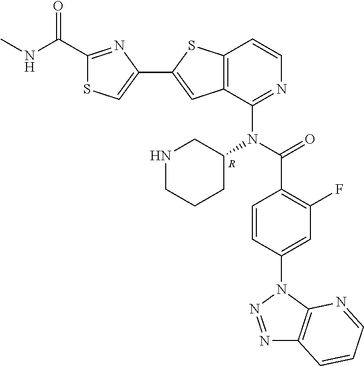 |
| 528 | (R)-N-(2-(4- (dimethylcarbamoyl)phenyl)thieno[3,2- c]pyridin-4-yl)-2-fluoro-4-(1-methyl-1H- 1,2,3-triazol-4-yl)-N-(piperidin-3- yl)benzamide | 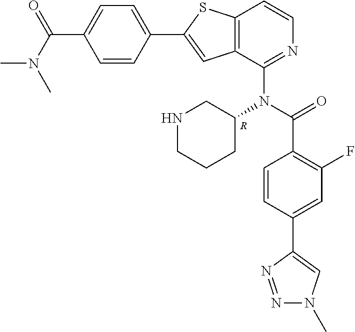 |
| 529 | 2-fluoro-4-(1-methyltriazol-4-yl)-N-[(3R)- 3-piperidyl]-N-[2-[4-(1H-pyrazol-3-yl) phenyl] thieno [3, 2-c] pyridin-4-yl] benzamide | 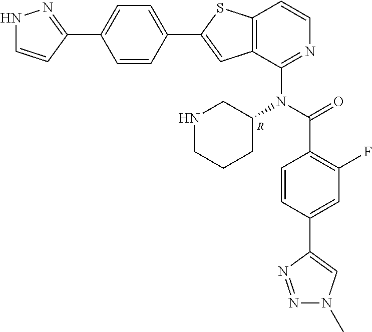 |
| 530 | (R)-4-(3H-[1,2,3]triazolo[4,5-b]pyridin-3- yl)-2-fluoro-N-(6-(5-(hydroxymethyl)-1H- 1,2,3-triazol-4-yl)isoquinolin-1-yl)-N- (piperidin-3-yl)benzamide | 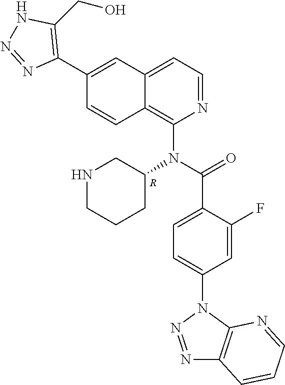 |
| 531 | (R)-2-fluoro-4-(1-methyl-1H-1,2,3-triazol- 4-yl)-N-(2-(2-(methylamino)pyrimidin-4- yl)thieno[3,2-c]pyridin-4-yl)-N-(piperidin- 3-yl)benzamide | 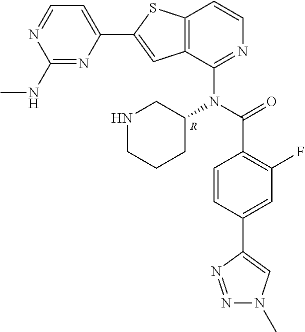 |
| 532 | 2-fluoro-4-(1-methyltriazol-4-yl)-N-[(3R)- 3-piperidyl]-N-[2-[3-(prop-2- enoylamino)phenyl]thieno[3,2-c]pyridin-4- yl]benzamide | 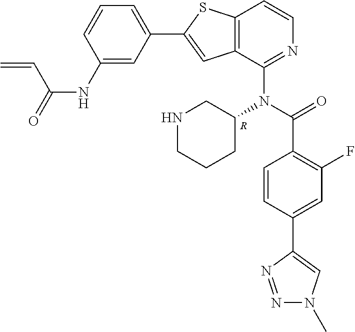 |
| 533 | 2-fluoro-4-(1-methyltriazol-4-yl)-N-[2-(3- oxoisoindolin-5-yl)thieno[3,2-c]pyridin-4- yl]-N-[(3R)-3-piperidyl]benzamide | 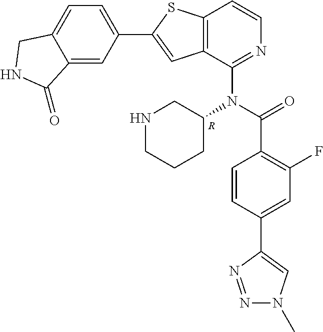 |
| 534 | (R)-2-fluoro-4-(1-methyl-1H-1,2,3-triazol- 4-yl)-N-(2-(4-(N- methylsulfamoyl)phenyl)thieno[3,2- c]pyridin-4-yl)-N-(piperidin-3- yl)benzamide | 1574 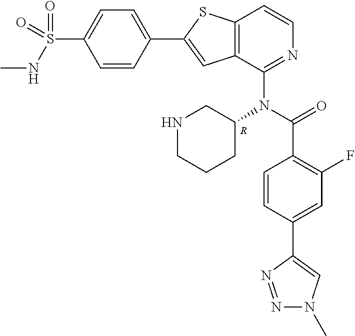 |
| 535 | 2-fluoro-N-[2-[3- (methylsulfamoyl)phenyl]thieno[3,2- c]pyridin-4-yl]-4-(1-methyltriazol-4-yl)-N- [(3R)-3-piperidyl]benzamide | 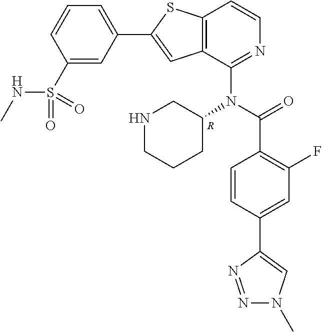 |
| 536 | N-[2-[4-(2-aminoethylsulfamoyl) phenyl]thieno[3,2-c]pyridin-4-yl]-2-fluoro- 4-(1-methyltriazol-4-yl)-N-[(3R)-3- piperidyl] benzamide | 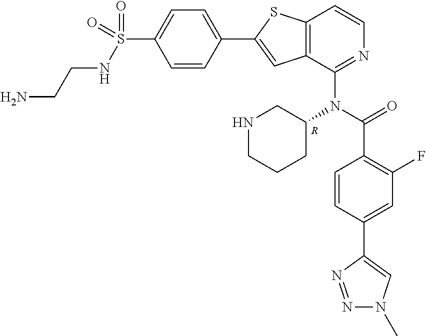 |
| 537 | 2-fluoro-N-[2-[4- (isopropylsulfamoyl)phenyl]thieno[3,2- c]pyridin-4-yl]-4-(1-methyltriazol-4-yl)-N- [(3R)-3-piperidyl]benzamide | 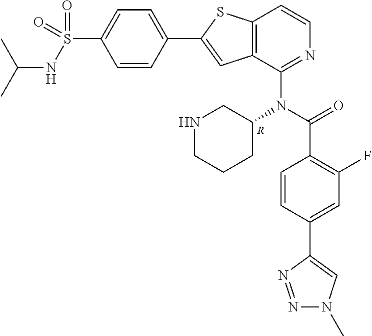 |
| 538 | 2-fluoro-4-(1-methyltriazol-4-yl)-N-[2-(1- oxoisoindolin-5-yl)thieno[3,2-c]pyridin-4- yl]-N-[(3R)-3-piperidyl]benzamide | 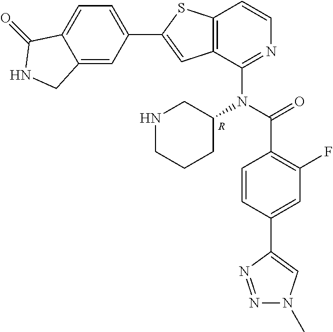 |
| 539 | 2-fluoro-N-[2-[3- (isopropylcarbamoyl)phenyl]thieno[3,2- c]pyridin-4-yl]-4-(1-methyltriazol-4-yl)-N- [(3R)-3-piperidyl]benzamide | 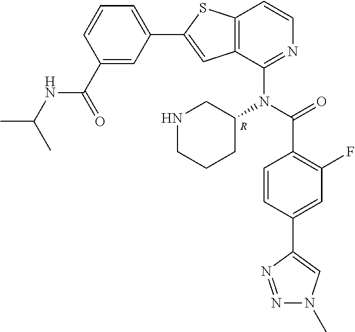 |
| 540 | 2-fluoro-4-(1-methyltriazol-4-yl)-N-[2-[3- (phenylcarbamoyl)phenyl]thieno[3,2- c]pyridin-4-yl]-N-[(3R)-3- piperidyl]benzamide | 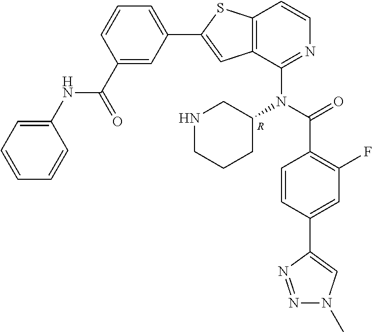 |
| 541 | 2-fluoro-N-[2-[3-(methylcarbamoyl) phenyl]thieno[3, 2-c] pyridin-4-yl]-4-(1- methyltriazol-4-yl)-N-[(3R)-3-piperidyl] benzamide | 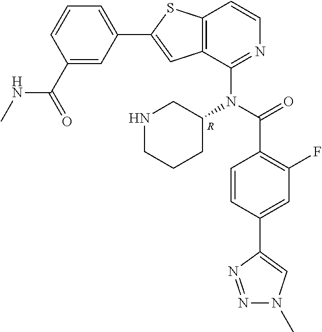 |
| 542 | 2-fluoro-N-[2-[3-(2- hydroxyethylcarbamoyl)phenyl]thieno[3,2- c]pyridin-4-yl]-4-(1-methyltriazol-4-yl)-N- [(3R)-3-piperidyl]benzamide | 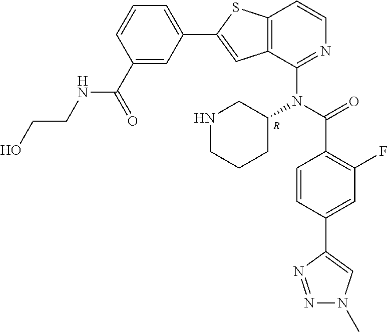 |
| 543 | 2-fluoro-N-[2-(1H-indol-6-yl) thieno [3, 2- c] pyridin-4-yl]-4-(1-methyltriazol-4-yl)- N-[(3R)-3-piperidyl] benzamide | 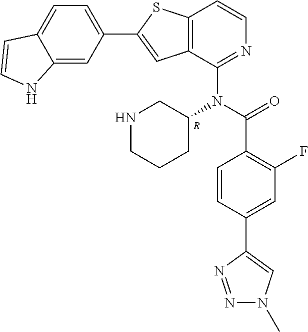 |
| 544 | 2-fluoro-N-[2-(1H-indol-6-yl) thieno [3, 2- c] pyridin-4-yl]-4-(1-methyltriazol-4-yl)- N-[(3R)-3-piperidyl] benzamide | 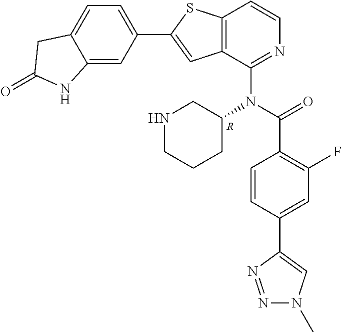 |
| 545 | (R)-2-fluoro-4-(1-methyl-1H-1,2,3-triazol- 4-yl)-N-(2-(2-oxo-2,3-dihydro-1H- benzo[d]imidazol-5-yl)thieno[3,2- c]pyridin-4-yl)-N-(piperidin-3- yl)benzamide | 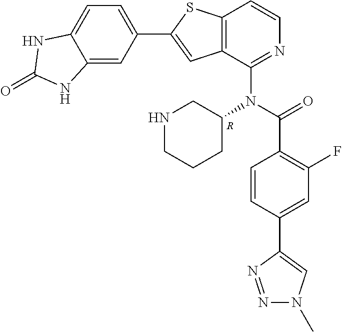 |
| 546 | 2-fluoro-N-[2-(1H-indol-5-yl)thieno[3,2- c]pyridin-4-yl]-4-(1-methyltriazol-4-yl)-N- [(3R)-3-piperidyl]benzamide | 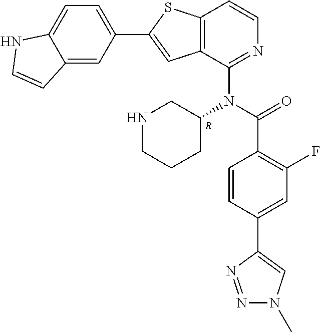 |
| 547 | (R)-N-(2-(4-acetamidophenyl)thieno[3,2- c]pyridin-4-yl)-2-fluoro-4-(1-methyl-1H- 1,2,3-triazol-4-yl)-N-(piperidin-3- yl)benzamide | 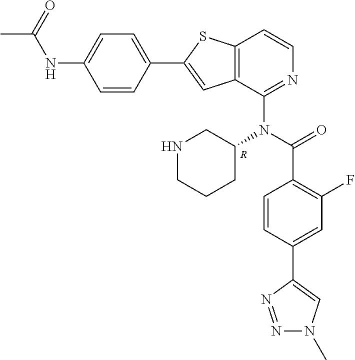 |
| 548 | (R)-2-fluoro-4-(1-methyl-1H-1,2,3-triazol- 4-yl)-N-(2-(4-(3- methylthioureido)phenyl)thieno[3,2- c]pyridin-4-yl)-N-(piperidin-3- yl)benzamide | 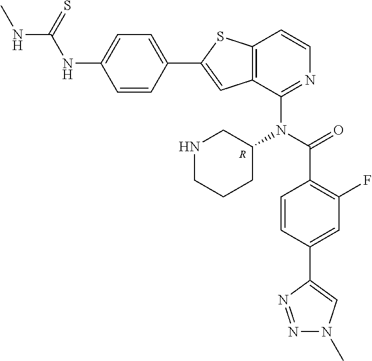 |
| 549 | 2-fluoro-N-[2-[3-(methylamino)-3-oxo- prop-1-ynyl]thieno[3,2-c]pyridin-4-yl]-N- [(3R)-3-piperidyl]-4-(triazolo [4,5- b]pyridin-3-yl)benzamide | 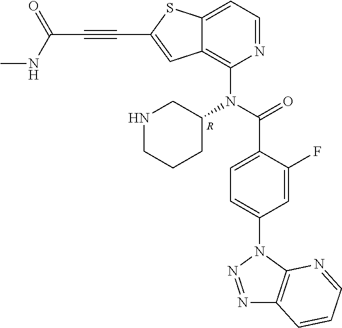 |
| 550 | 2-fluoro-N-[2-[3-(methylamino)-3-oxo- prop-1-ynyl]thieno[3,2-c]pyridin-4-yl]-4- (1-methyltriazol-4-yl)-N-[(3R)-3-piperidyl] benzamide | 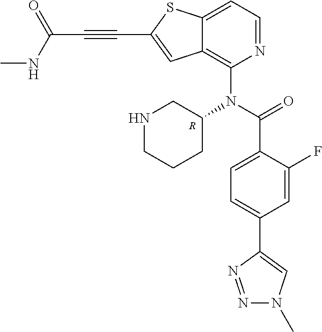 |
| 551 | 2-fluoro-N-[2-(3-hydroxy-3-methyl-but-1- ynyl)thieno[3,2-c]pyridin-4-yl]-4-(1- methyltriazol-4-yl)-N-[(3R)-3- piperidyl]benzamide | 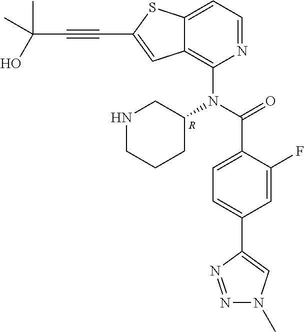 |
| 552 | 4-[[2-fluoro-4-(1-methyltriazol-4-yl) benzoyl]-[(3R)-3-piperidyl] amino] thieno [3, 2-c] pyridine-2-carboxamide | 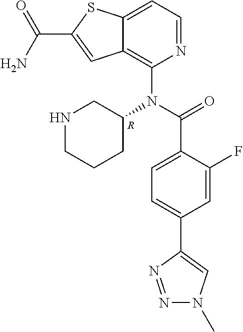 |
| 553 | N-[2-(6-amino-3-pyridyl)thieno[3,2- c]pyridin-4-yl]-2-fluoro-N-[(3R)-3- piperidyl]-4-(triazolo[4,5-b] pyridin-3- yl)benzamide | 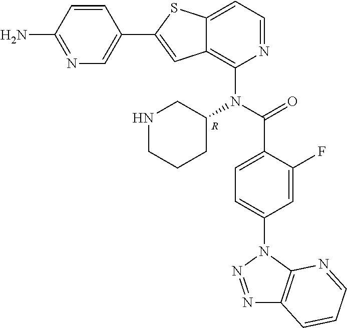 |
| 556 | (R)-4-(3H-[1,2,3] triazolo[4,5-b]pyridin-3- yl)-2-fluoro-N-(piperidin-3-yl)-N-(6- (pyridin-2-yl)isoquinolin-1-yl) benzamide | 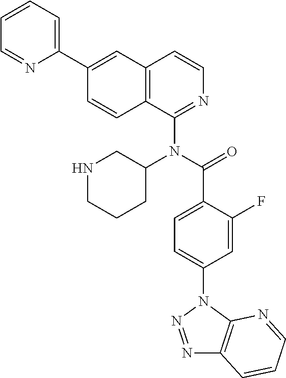 [SMILES: O=C(c1ccc(-n2nnc3cccnc32)cc1F)N(c1nccc2cc(-c3ccccn3)ccc12)C1CCCNC1] |
| 557 | 2-fluoro-N-[2-(1H-indazol-5-yl)thieno[3,2- c]pyridin-4-yl]-N-[(3R)-3-piperidyl]-4- (triazolo[4,5-b]pyridin-3-yl)benzamide | 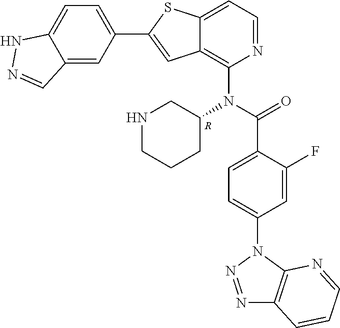 |
| 558 | (R)-2-fluoro-N-(8-methylisoquinolin-1-yl)- N-(piperidin-3-yl)-4-((4-(pyridin-4- yl)pyrimidin-2-yl)amino)benzamide | 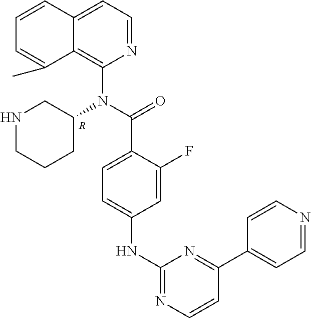 |
| 559 | (R)-2-fluoro-N-(8-methylisoquinolin-1-yl)- 4-((4-phenylpyrimidin-2-yl)amino)-N- (piperidin-3-yl)benzamide | 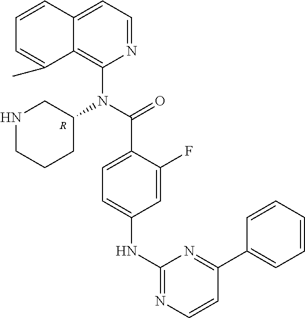 |
| 560 | (R)-2-fluoro-N-(6-fluoroisoquinolin-1-yl)- 4-((4-morpholinopyrimidin-2-yl)amino)-N- (piperidin-3-yl)benzamide | 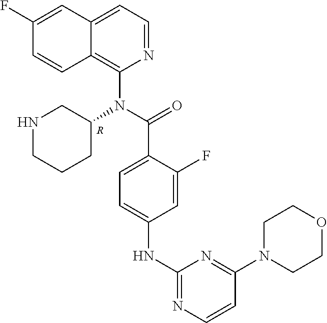 |
| 561 | (R)-2-fluoro-N-(6-fluoroisoquinolin-1-yl)- 4-((4-(piperidin-1-yl)pyrimidin-2- yl)amino)-N-(piperidin-3-yl)benzamide | 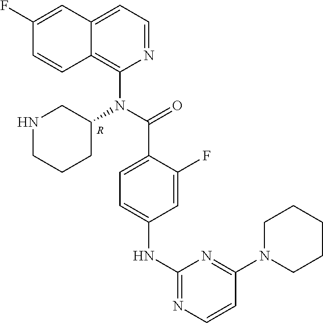 |
| 562 | (R)-2-fluoro-N-(6-fluoroisoquinolin-1-yl)- 4-((4-(4-methylpiperazin-1-yl)pyrimidin-2- yl)amino)-N-(piperidin-3-yl)benzamide | 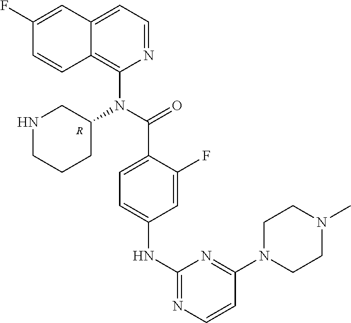 |
| 563 | (R)-2-fluoro-4-(1-methyl-1H-1,2,3-triazol- 4-yl)-N-(2-nicotinoylthieno[3,2-c]pyridin- 4-yl)-N-(piperidin-3-yl)benzamide | 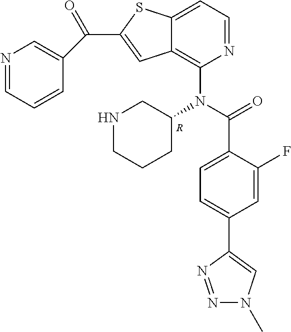 |
| 564 | (R)-2-fluoro-N-(6-fluoro-8- methylisoquinolin-1-yl)-N-(piperidin-3- yl)-4-(pyrimidin-2-ylamino)benzamide | 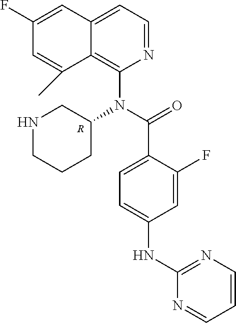 |
| 565 | N-[2-(4-amino-3,5-difluoro- phenyl)thieno[3,2-c]pyridin-4-yl]-2-fluoro- N-[(3R)-3-piperidyl]-4-(triazolo[4,5- b]pyridin-3-yl)benzamide | 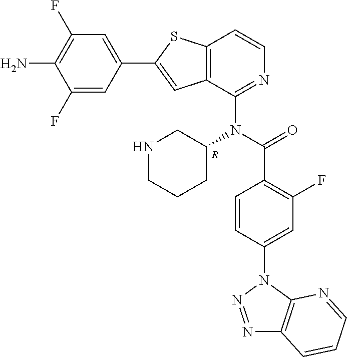 |
| 566 | 2-fluoro-N-[2-(2-fluoro-4-pyridyl) thieno [3, 2-c] pyridin-4-yl]-N-[(3R)-3-piperidyl]- 4-(triazolo [4, 5-b] pyridin-3-yl) benzamide | 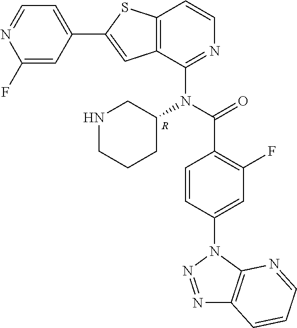 |
| 567 | 2-fluoro-4-[4-[[2-fluoro-4-(triazolo[4,5- b]pyridin-3-yl)benzoyl]-[(3R)-3- piperidyl]amino] thieno[3,2-c]pyridin-2- yl]benzamide | 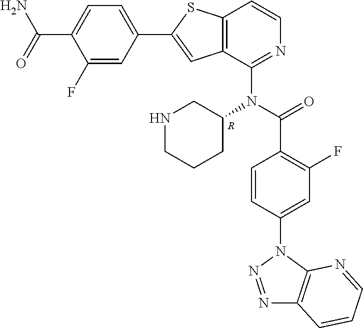 |
| 568 | 4-[4-[[2-fluoro-4-(triazolo[4,5-b]pyridin-3- yl)benzoyl]-[(3R)-3- piperidyl]amino]thieno[3,2-c]pyridin-2- yl]thiophene-2-carboxamide | 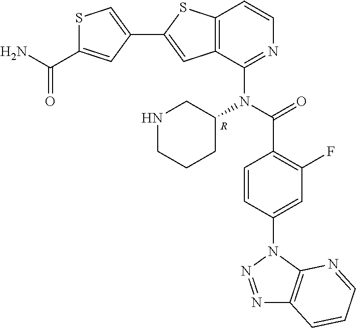 |
| 569 | [4-[4-[[2-fluoro-4-(triazolo[4,5-b]pyridin- 3-yl)benzoyl]-[(3R)-3- piperidyl]amino]thieno[3,2-c]pyridin-2- yl]phenyl]boronic acid | 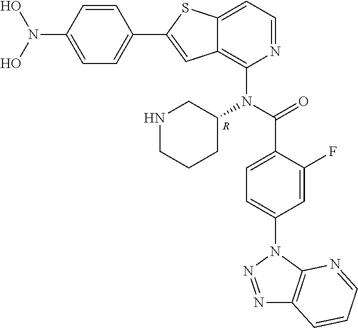 |
| 570 | N-[2-[4-(2-amino-2-oxo- ethyl)phenyl]thieno[3,2-c]pyridin-4-yl]-2- fluoro-N-[(3R)-3-piperidyl]-4- (triazolo[4,5-b]pyridin-3-yl)benzamide | 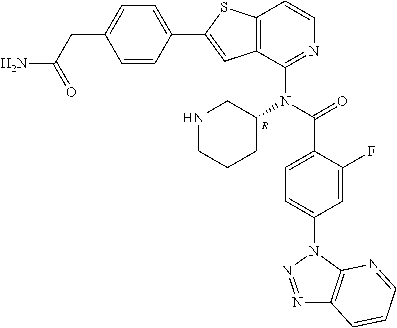 |
| 571 | N-[2-(2,6-dimethyl-4-pyridyl)thieno[3,2- c]pyridin-4-yl]-2-fluoro-N-[(3R)-3- piperidyl]-4-(triazolo[4,5-b] pyridin-3- yl)benzamide | 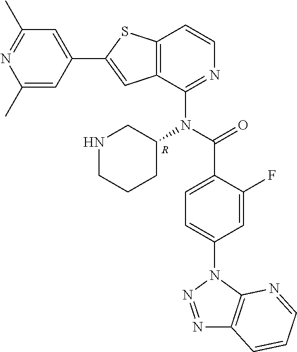 |
| 572 | 4-[4-[[2-fluoro-4-(triazolo [4, 5-b] pyridin- 3-yl) benzoyl]-[(3R)-3-piperidyl] amino] thieno [3, 2-c] pyridin-2-yl]-2-methyl- benzamide | 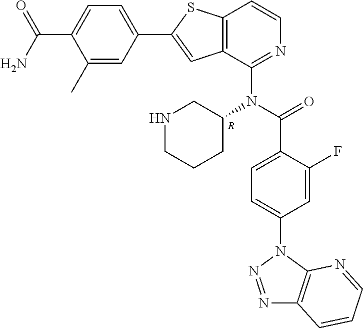 |
| 573 | 4-[4-[[2-fluoro-4-(triazolo [4, 5-b] pyridin- 3-yl) benzoyl]-[(3R)-3-piperidyl] amino] thieno [3, 2-c] pyridin-2-yl]-2- (trifluoromethyl) benzamide | 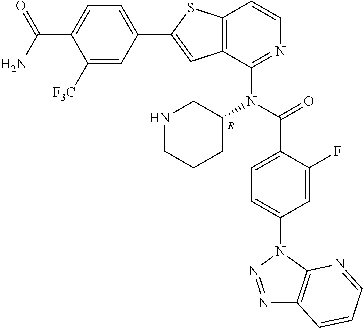 |
| 574 | (R)-4-(3H-[1,2,3]triazolo[4,5-b]pyridin-3- yl)-2-fluoro-N-(6-(5-methylthiazol-2- yl)isoquinolin-1-yl)-N-(piperidin-3- yl)benzamide | 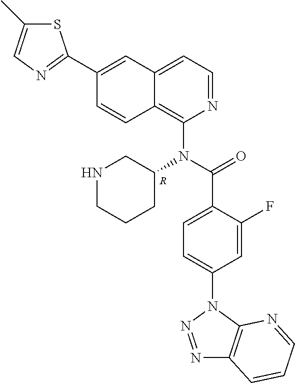 |
| 575 | (R)-4-(3H-[1,2,3]triazolo[4,5-b]pyridin-3- yl)-2-fluoro-N-(6-(2-methylthiazol-4- yl)isoquinolin-1-yl)-N-(piperidin-3- yl)benzamide | 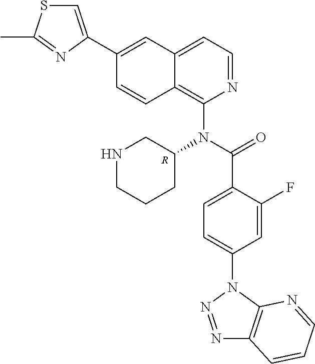 |
| 576 | (R)-4-(3H-[1,2,3]triazolo[4,5-b]pyridin-3- yl)-2-fluoro-N-(6-(5-methyl-1,3,4- thiadiazol-2-yl)isoquinolin-1-yl)-N- (piperidin-3-yl)benzamide | 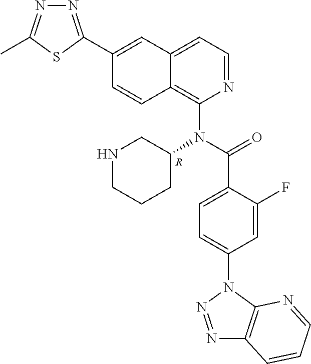 |
| 577 | (R)-2-fluoro-4-(1-methyl-1H-1,2,3-triazol- 4-yl)-N-(6-phenylisoquinolin-1-yl)-N- (piperidin-3-yl)benzamide | 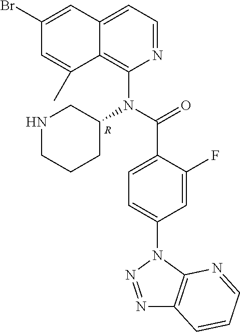 |
| 578 | (R)-2-fluoro-N-(8-methylisoquinolin-1-yl)- 4-((4-(piperidin-1-yl)pyrimidin-2- yl)amino)-N-(piperidin-3-yl)benzamide | 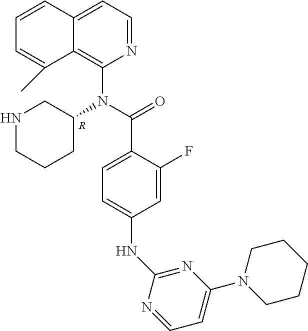 |
| 579 | (R)-2-fluoro-4-(1-methyl-1H-1,2,3-triazol- 4-yl)-N-(6-phenylisoquinolin-1-yl)-N- (piperidin-3-yl)benzamide | 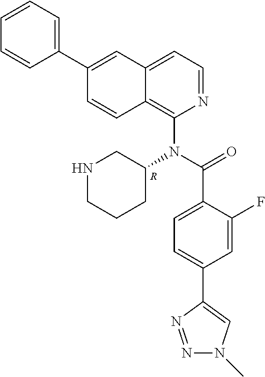 |
| 580 | (R)-2-fluoro-N-(6-(4- fluorophenyl)isoquinolin-1-yl)-4-(1- methyl-1H-1,2,3-triazol-4-yl)-N- (piperidin-3-yl)benzamide | 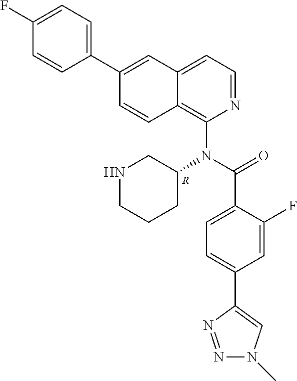 |
| 581 | (R)-2-fluoro-4-(1-methyl-1H-1,2,3-triazol- 4-yl)-N-(piperidin-3-yl)-N-(6-(pyridin-2- yl)isoquinolin-1-yl)benzamide | 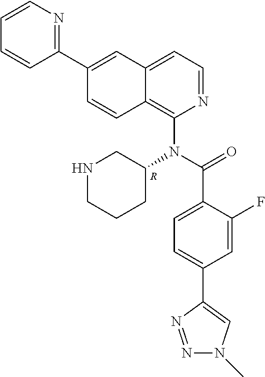 |
| 582 | (R)-2-fluoro-4-(1-methyl-1H-1,2,3-triazol- 4-yl)-N-(6-(4- (methylsulfonyl)phenyl)isoquinolin-1-yl)- N-(piperidin-3-yl)benzamide | 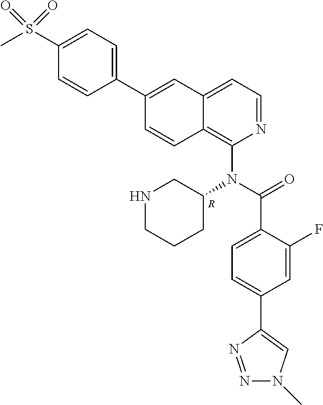 |
| 583 | (R)-4-(3H-[1,2,3]triazolo[4,5-b]pyridin-3- yl)-2-fluoro-N-(6-fluoro-8- methylisoquinolin-1-yl)-N-(piperidin-3- yl)benzamide | 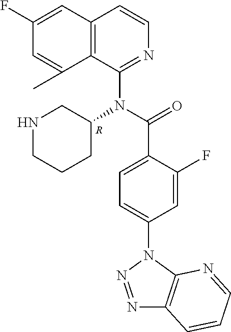 |
| 584 | (R)-4-(4-(4-(3H-[1,2,3]triazolo[4,5- b]pyridin-3-yl)-2-fluoro-N-(piperidin-3- yl)benzamido)thieno[3,2-c]pyridin-2-yl)- 2,6-difluorobenzoic acid | 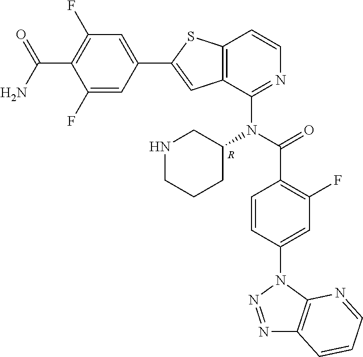 |
| 585 | (R)-4-(3H-[1,2,3]triazolo[4,5-b]pyridin-3- yl)-2-fluoro-N-(2-(4-(2-hydroxypropan-2- yl)phenyl)thieno[3,2-c]pyridin-4-yl)-N- (piperidin-3-yl)benzamide | 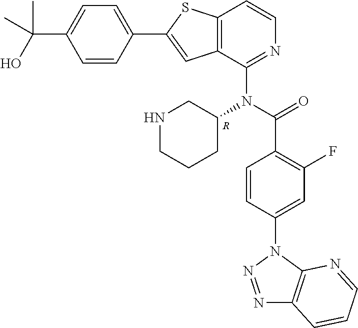 |
| 586 | (R)-4-(3H-[1,2,3]triazolo[4,5-b]pyridin-3- yl)-2-fluoro-N-(6-(3-hydroxy-3-methylbut- 1-yn-1-yl)isoquinolin-1-yl)-N-(piperidin-3- yl)benzamide | 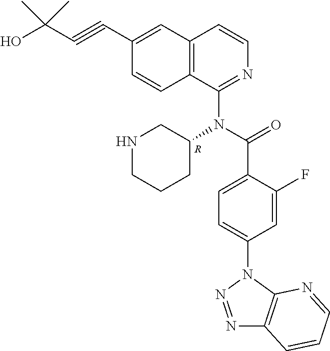 |
| 587 | (R)-4-(3H-[1,2,3]triazolo[4,5-b]pyridin-3- yl)-2-fluoro-N-(6-(3-hydroxy-3-methylbut- 1-yn-1-yl)-8-methylisoquinolin-1-yl)-N- (piperidin-3-yl)benzamide | 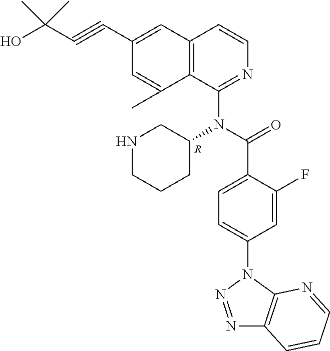 |
| 588 | (R)-3-(4-(1,3-dioxoisoindolin-2-yl)but-1- yn-1-yl)-N-(8-methylisoquinolin-1-yl)-N- (piperidin-3-yl)benzamide | 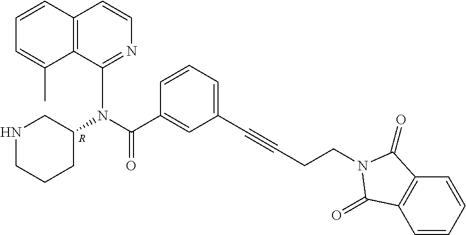 |
| 589 | methyl (R)-5-(3-((8-methylisoquinolin-1- yl)(piperidin-3-yl)carbamoyl)phenyl)pent- 4-ynoate | 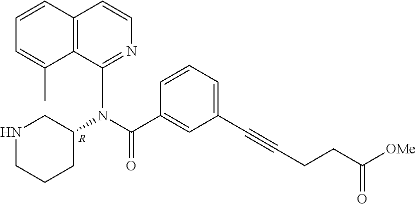 |
| 590 | (R)-5-(3-((8-methylisoquinolin-1- yl)(piperidin-3-yl)carbamoyl)phenyl)pent- 4-ynoic acid | 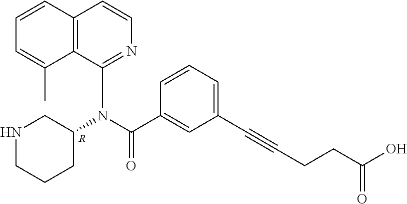 |
| 591 | (R)-2-fluoro-N-(6-fluoroisoquinolin-1-yl)- N-(piperidin-3-yl)-4-(pyrimidin-2- ylamino)benzamide | 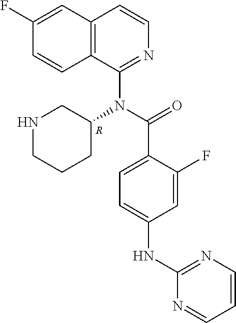 |
| 592 | (R)-2-fluoro-4-(3-hydroxy-3-methylbut-1- yn-1-yl)-N-(8-methylisoquinolin-1-yl)-N- (piperidin-3-yl)benzamide | 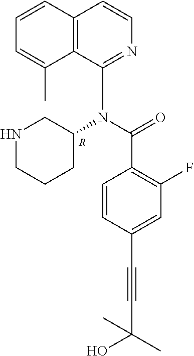 |
| 593 | 2-fluoro-4-(5-methyl-1,3,4-thiadiazol-2- yl)-N-[(3R)-3-piperidyl]-N-[2-(4- pyridyl)thieno[3,2-c]pyridin-4- yl]benzamide | 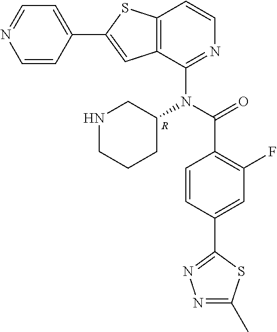 |
| 594 | (R)-4-(3H-[1,2,3]triazolo[4,5-b]pyridin-3- yl)-N-(2-(3-((1-amino-2-methyl-1- oxopropan-2-yl)oxy)phenyl)thieno[3,2- c]pyridin-4-yl)-2-fluoro-N-(piperidin-3- yl)benzamide | 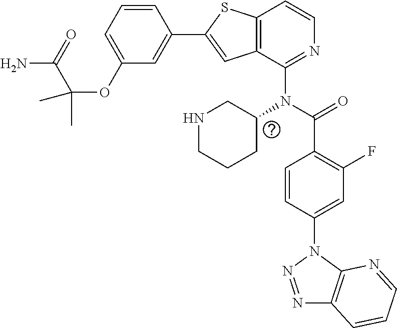 [SMILES: CC(C)(Oc1cccc(-c2cc3c(N(C(=O)c4ccc(-n5nnc6cccnc65)cc4F)[C@@H]4CCCNC4)nccc3s2)c1)C(N)=O] |
| 595 | (R)-4-(3H-[1,2,3]triazolo[4,5-b]pyridin-3- yl)-N-(2-(4-(1-amino-2-methyl-1- oxopropan-2-yl)phenyl)thieno[3,2- c]pyridin-4-yl)-2-fluoro-N-(piperidin-3- yl)benzamide | 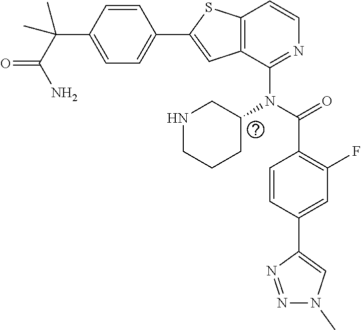 [SMILES: Cn1cc(-c2ccc(C(=O)N(c3nccc4sc(-c5ccc(C(C)(C)C(N)=O)cc5)cc34)[C@@H]3CCCNC3)c(F)c2)nn1] |
| 596 | (R)-4-(3H-[1,2,3]triazolo[4,5-b]pyridin-3- yl)-N-(2-(3-(1-amino-2-methyl-1- oxopropan-2-yl)phenyl)thieno[3,2- c]pyridin-4-yl)-2-fluoro-N-(piperidin-3- yl)benzamide | 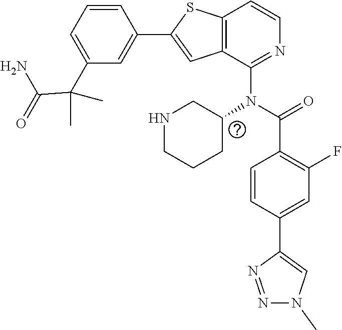 [SMILES: Cn1cc(-c2ccc(C(=O)N(c3nccc4sc(-c5cccc(C(C)(C)C(N)=O)c5)cc34)[C@@H]3CCCNC3)c(F)c2)nn1] |
| 597 | (R)-N-(2-(4-(1H-pyrazol-4- yl)phenyl)thieno[3,2-c]pyridin-4-yl)-4- (3H-[1,2,3]triazolo[4,5-b]pyridin-3-yl)-2- fluoro-N-(piperidin-3-yl)benzamide | 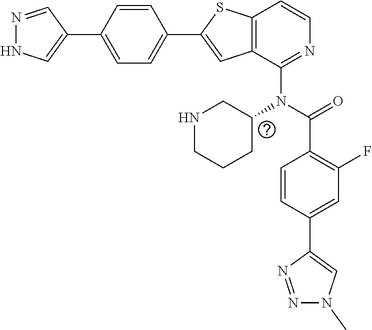 [SMILES: Cn1cc(-c2ccc(C(=O)N(c3nccc4sc(-c5ccc(-c6cn[nH]c6)cc5)cc34)[C@@H]3CCCNC3)c(F)c2)nn1] |
| 598 | (R)-4-(3H-[1,2,3]triazolo[4,5-b]pyridin-3- yl)-2-fluoro-N-(2-(4- fluorophenyl)thieno[3,2-c]pyridin-4-yl)-N- (piperidin-3-yl)benzamide | 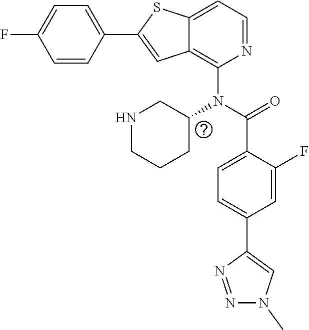 [SMILES: Cn1cc(-c2ccc(C(=O)N(c3nccc4sc(-c5ccc(F)cc5)cc34)[C@@H]3CCCNC3)c(F)c2)nn1] |
| 599 | (R)-4-(3H-[1,2,3]triazolo[4,5-b]pyridin-3- yl)-2-fluoro-N-(2-(4- (hydroxymethyl)phenyl)thieno[3,2- c]pyridin-4-yl)-N-(piperidin-3- yl)benzamide | 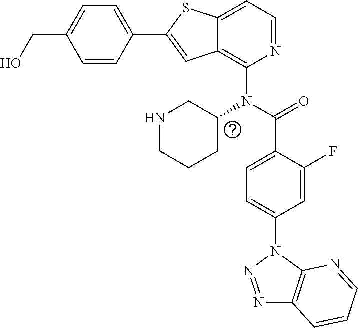 [SMILES: O=C(c1ccc(-n2nnc3cccnc32)cc1F)N(c1nccc2sc(-c3ccc(CO)cc3)cc12)[C@@H]1CCCNC1] |
| 600 | 4-(3H-[1,2,3]triazolo[4,5-b]pyridin-3-yl)- 2-fluoro-N-(2-(4-(1- hydroxyethyl)phenyl)thieno[3,2-c]pyridin- 4-yl)-N-((R)-piperidin-3-yl)benzamide | 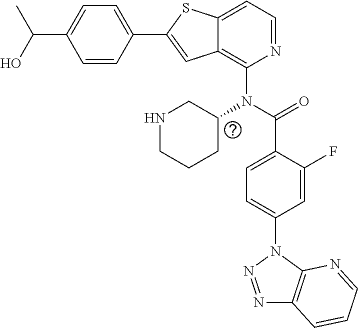 [SMILES: CC(O)c1ccc(-c2cc3c(N(C(=O)c4ccc(-n5nnc6cccnc65)cc4F)[C@@H]4CCCNC4)nccc3s2)cc1] |
| 601 | 4-(3H-[1,2,3]triazolo[4,5-b]pyridin-3-yl)- 2-fluoro-N-(2-(3-(1- hydroxyethyl)phenyl)thieno[3,2-c]pyridin- 4-yl)-N-((R)-piperidin-3-yl)benzamide | 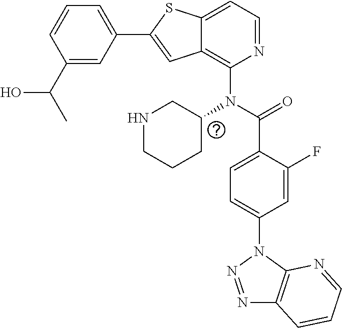 [SMILES: CC(O)c1cccc(-c2cc3c(N(C(=O)c4ccc(-n5nnc6cccnc65)cc4F)[C@@H]4CCCNC4)nccc3s2)c1] |
| 602 | (R)-4-(3H-[1,2,3]triazolo[4,5-b]pyridin-3- yl)-2-fluoro-N-(2-(3-(2-hydroxypropan-2- yl)phenyl)thieno[3,2-c]pyridin-4-yl)-N- (piperidin-3-yl)benzamide | 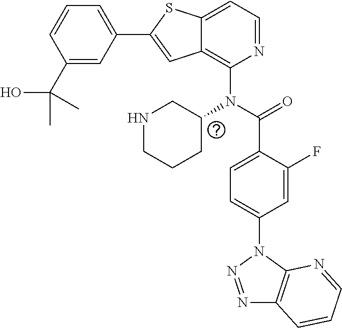 [SMILES: CC(C)(O)c1cccc(-c2cc3c(N(C(=O)c4ccc(-n5nnc6cccnc65)cc4F)[C@@H]4CCCNC4)nccc3s2)c1] |
| 603 | N-[2-(4-acetylphenyl) thieno [3,2- c]pyridin-4-yl]-2-fluoro-N-[(3R)-3- piperidyl]-4-(triazolo[4,5-b]pyridin-3-yl) benzamide | 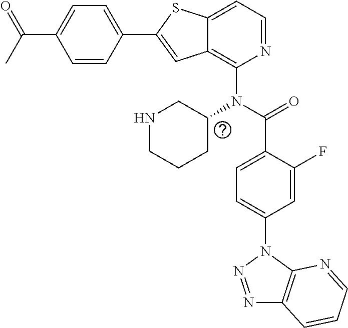 [SMILES: CC(=O)c1ccc(-c2cc3c(N(C(=O)c4ccc(-n5nnc6cccnc65)cc4F)[C@@H]4CCCNC4)nccc3s2)cc1] |
| 604 | (R)-2-fluoro-N-(2-(4-(1,1,1,3,3,3- hexafluoro-2-hydroxypropan-2- yl)phenyl)thieno[3,2-c]pyridin-4-yl)-4-(1- methyl-1H-1,2,3-triazol-4-yl)-N- (piperidin-3-yl)benzamide | 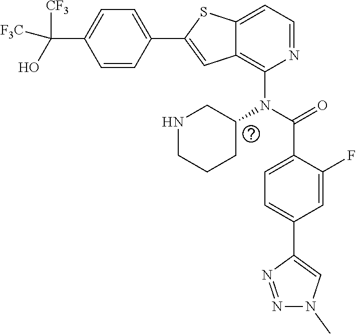 [SMILES: Cn1cc(-c2ccc(C(=O)N(c3nccc4sc(-c5ccc(C(O)(C(F)(F)F)C(F)(F)F)cc5)cc34)[C@@H]3CCCNC3)c(F)c2)nn1] |
| 605 | 2-fluoro-4-(1-methyl-1H-1,2,3-triazol-4- yl)-N-((R)-piperidin-3-yl)-N-(2-(4-(2,2,2- trifluoro-1- hydroxyethyl)phenyl)thieno[3,2-c]pyridin- 4-yl)benzamide | 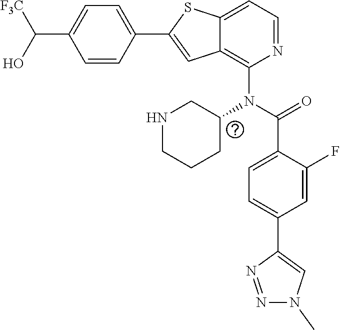 [SMILES: Cn1cc(-c2ccc(C(=O)N(c3nccc4sc(-c5ccc(C(O)C(F)(F)F)cc5)cc34)[C@@H]3CCCNC3)c(F)c2)nn1] |
| 606 | (R)-4-(3H-[1,2,3]triazolo[4,5-b]pyridin-3- yl)-N-(2-(4-carbamoyl-3,5- dimethylphenyl)thieno[3,2-c]pyridin-4-yl)- 2-fluoro-N-(piperidin-3-yl)benzamide | 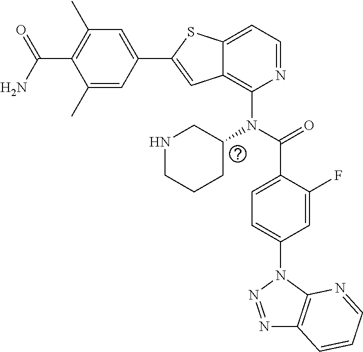 [SMILES: Cc1cc(-c2cc3c(N(C(=O)c4ccc(-n5nnc6cccnc65)cc4F)[C@@H]4CCCNC4)nccc3s2)cc(C)c1C(N)=O] |
| 607 | (R)-N-(2-(4-carbamoyl-3- methylphenyl)thieno[3,2-c]pyridin-4-yl)-2- fluoro-4-(1-methyl-1H-1,2,3-triazol-4-yl)- N-(piperidin-3-yl)benzamide | 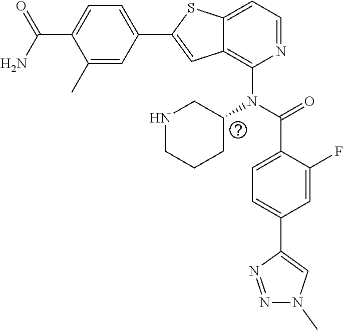 [SMILES: Cc1cc(-c2cc3c(N(C(=O)c4ccc(-c5cn(C)nn5)cc4F)[C@@H]4CCCNC4)nccc3s2)ccc1C(N)=O] |
| 608 | (R)-N-(2-(4-carbamoyl-3,5- dimethylphenyl)thieno[3,2-c]pyridin-4-yl)- 2-fluoro-4-(1-methyl-1H-1,2,3-triazol-4- yl)-N-(piperidin-3-yl)benzamide | 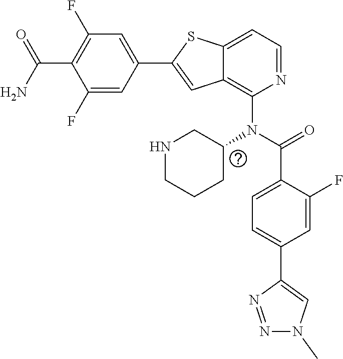 [SMILES: Cn1cc(-c2ccc(C(=O)N(c3nccc4sc(-c5cc(F)c(C(N)=O)c(F)c5)cc34)[C@@H]3CCCNC3)c(F)c2)nn1] |
| 609 | (R)-N-(6-benzylisoquinolin-1-yl)-2-fluoro- 4-(1-methyl-1H-1,2,3-triazol-4-yl)-N- (piperidin-3-yl)benzamide | 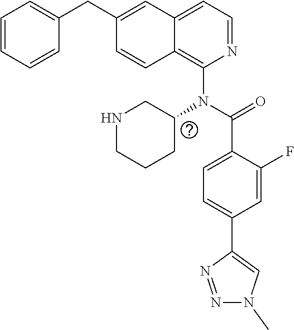 [SMILES: Cn1cc(-c2ccc(C(=O)N(c3nccc4cc(Cc5ccccc5)ccc34)[C@@H]3CCCNC3)c(F)c2)nn1] |
| 610 | (R)-N-(6-acetylisoquinolin-1-yl)-2-fluoro- 4-(1-methyl-1H-1,2,3-triazol-4-yl)-N- (piperidin-3-yl)benzamide | 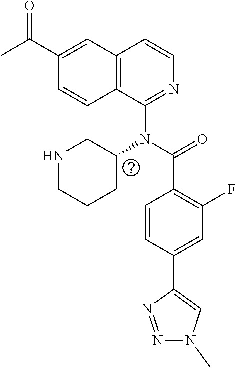 [SMILES: CC(=O)c1ccc2c(N(C(=O)c3ccc(-c4cn(C)nn4)cc3F)[C@@H]3CCCNC3)nccc2c1] |
| 611 | (R)-N-(2-(4-carbamoyl-3,5- dimethylphenyl)thieno[3,2-c]pyridin-4-yl)- 2-fluoro-4-(1-methyl-1H-1,2,3-triazol-4- yl)-N-(piperidin-3-yl)benzamide | 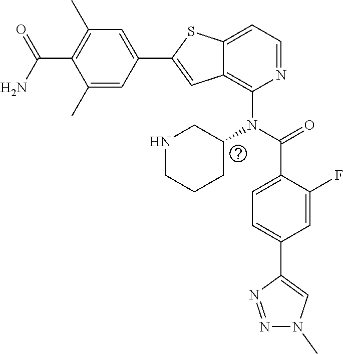 [SMILES: Cc1cc(-c2cc3c(N(C(=O)c4ccc(-c5cn(C)nn5)cc4F)[C@@H]4CCCNC4)nccc3s2)cc(C)c1C(N)=O] |
| 612 | (R)-N-(2-(4-carbamoylphenyl)thieno[3,2- c]pyridin-4-yl)-4-(1,5-dimethyl-1H- pyrazol-4-yl)-2-fluoro-N-(piperidin-3- yl)benzamide | 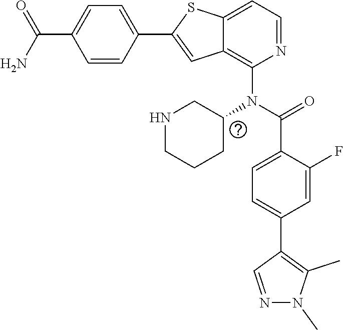 [SMILES: Cc1c(-c2ccc(C(=O)N(c3nccc4sc(-c5ccc(C(N)=O)cc5)cc34)[C@@H]3CCCNC3)c(F)c2)cnn1C] |
| 613 | N-(2-(4-(1-amino-2,2,2- trifluoroethyl)phenyl)thieno[3,2-c]pyridin- 4-yl)-2-fluoro-4-(1-methyl-1H-1,2,3- triazol-4-yl)-N-((R)-piperidin-3- yl)benzamide | 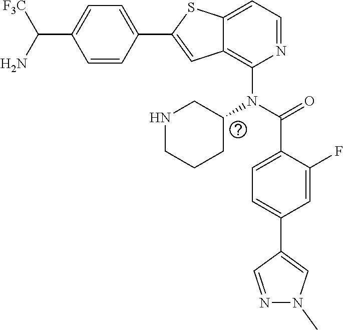 [SMILES: Cn1cc(-c2ccc(C(=O)N(c3nccc4sc(-c5ccc(C(N)C(F)(F)F)cc5)cc34)[C@@H]3CCCNC3)c(F)c2)cn1] |
| 614 | (R)-N-(6-fluoro-8-methylisoquinolin-1-yl)- 4-(1-methyl-1H-1,2,3-triazol-4-yl)-N- (piperidin-3-yl)benzamide | 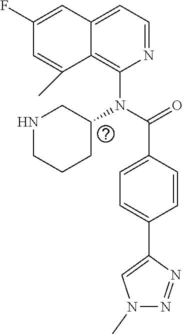 [SMILES: Cc1cc(F)cc2ccnc(N(C(=O)c3ccc(-c4cn(C)nn4)cc3)[C@@H]3CCCNC3)c12] |
| 615 | N-(6-chloro-8-methyl-1-isoquinolyl)-2- fluoro-N-[(3R)-3-piperidyl]-4-(triazolo[4,5- b]pyridin-3-yl)benzamide | 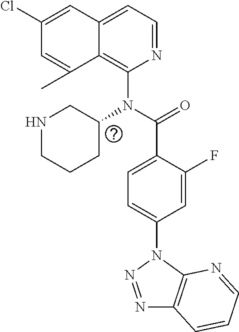 [SMILES: Cc1cc(Cl)cc2ccnc(N(C(=O)c3ccc(-n4nnc5cccnc54)cc3F)[C@@H]3CCCNC3)c12] |
| 617 | (R)-N-(3-ethylthieno[3,2-c]pyridin-4-yl)-2- fluoro-4-(5-methyl-1,3,4-thiadiazol-2-yl)- N-(piperidin-3-yl)benzamide | 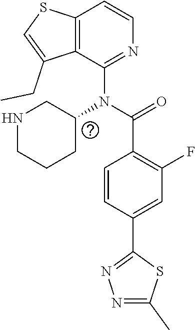 [SMILES: CCc1csc2ccnc(N(C(=O)c3ccc(-c4nnc(C)s4)cc3F)[C@@H]3CCCNC3)c12] |
| 618 | (R)-N-(6-chloro-8-methylisoquinolin-1- yl)-2-fluoro-4-(5-(hydroxymethyl)-1- methyl-1H-1,2,3-triazol-4-yl)-N- (piperidin-3-yl)benzamide | 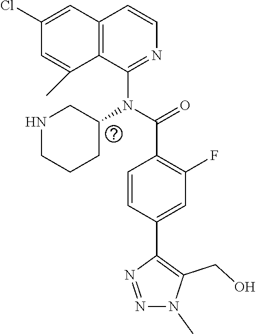 [SMILES: Cc1cc(Cl)cc2ccnc(N(C(=O)c3ccc(-c4nnn(C)c4CO)cc3F)[C@@H]3CCCNC3)c12] |
| 619 | (R)-2-fluoro-N-(6-methoxy-8- methylisoquinolin-1-yl)-4-(1-methyl-1H- 1,2,3-triazol-4-yl)-N-(piperidin-3- yl)benzamide | 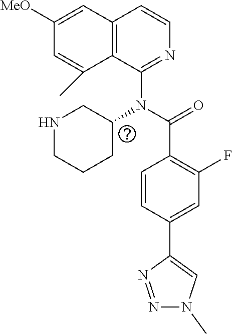 [SMILES: COc1cc(C)c2c(N(C(=O)c3ccc(-c4cn(C)nn4)cc3F)[C@@H]3CCCNC3)nccc2c1] |
| 620 | 2-fluoro-N-(6-fluoro-8-methyl-1- isoquinolyl)-4-(1-methyltriazol-4-yl)-N- [(3S)-3-piperidyl]benzamide | 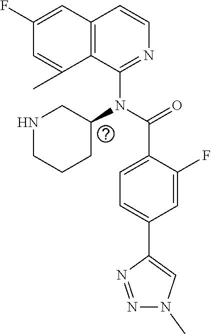 [SMILES: Cc1cc(F)cc2ccnc(N(C(=O)c3ccc(-c4cn(C)nn4)cc3F)[C@H]3CCCNC3)c12] |
| 621 | (S)-N-(6-chloro-8-methylisoquinolin-1-yl)- 2-fluoro-4-(1-methyl-1H-1,2,3-triazol-4- yl)-N-(piperidin-3-yl)benzamide | 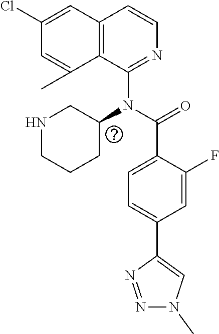 [SMILES: Cc1cc(Cl)cc2ccnc(N(C(=O)c3ccc(-c4cn(C)nn4)cc3F)[C@H]3CCCNC3)c12] |
| 622 | (R)-6-(5-methyl-1,3,4-thiadiazol-2-yl)-N- (8-methylisoquinolin-1-yl)-N-(piperidin-3- yl)nicotinamide | 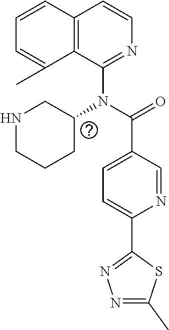 [SMILES: Cc1nnc(-c2ccc(C(=O)N(c3nccc4cccc(C)c34)[C@@H]3CCCNC3)cn2)s1] |
| 623 | 2-fluoro-4-((4-((R)-3-hydroxypiperidin-1- yl)pyrimidin-2-yl)amino)-N-(8- methylisoquinolin-1-yl)-N-((R)-piperidin- 3-yl)benzamide example | 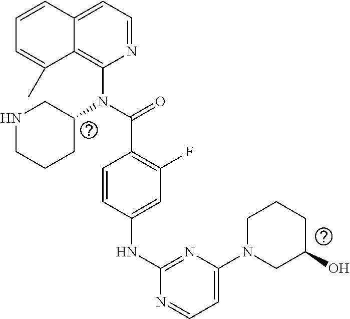 [SMILES: Cc1cccc2ccnc(N(C(=O)c3ccc(Nc4nccc(N5CCC[C@@H](O)C5)n4)cc3F)[C@@H]3CCCNC3)c12] |
| 624 | 2-fluoro-4-((4-((S)-3-hydroxypiperidin-1- yl)pyrimidin-2-yl)amino)-N-(8- methylisoquinolin-1-yl)-N-((R)-piperidin- 3-yl)benzamide | 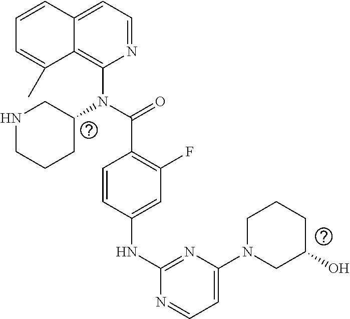 [SMILES: Cc1cccc2ccnc(N(C(=O)c3ccc(Nc4nccc(N5CCC[C@H](O)C5)n4)cc3F)[C@@H]3CCCNC3)c12] |
| 625 | 2-fluoro-4-((4-((S)-3-hydroxypyrrolidin-1- yl)pyrimidin-2-yl)amino)-N-(8- methylisoquinolin-1-yl)-N-((R)-piperidin- 3-yl)benzamide | 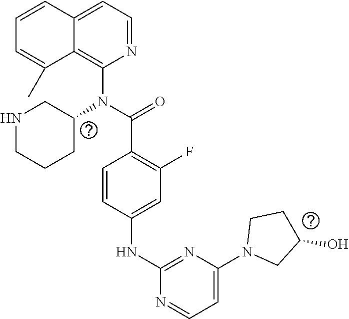 [SMILES: Cc1cccc2ccnc(N(C(=O)c3ccc(Nc4nccc(N5CC[C@H](O)C5)n4)cc3F)[C@@H]3CCCNC3)c12] |
| 626 | (R)-2-fluoro-N-(5-fluoro-8- methylisoquinolin-1-yl)-N-(piperidin-3- yl)-4-(pyrazolo[1,5-a]pyrimidin-3- yl)benzamide | 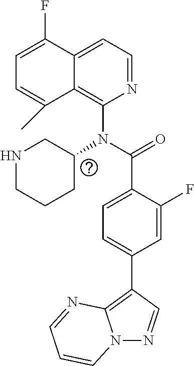 [SMILES: Cc1ccc(F)c2ccnc(N(C(=O)c3ccc(-c4cnn5cccnc45)cc3F)[C@@H]3CCCNC3)c12] |
| 627 | (R)-2-fluoro-N-(6-fluoro-8- methylisoquinolin-1-yl)-N-(piperidin-3- yl)-4-(pyrazolo[1,5-a]pyrimidin-3- yl)benzamide | 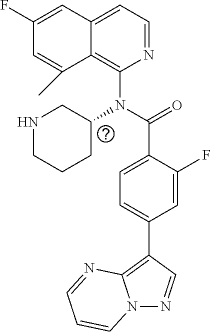 [SMILES: Cc1cc(F)cc2ccnc(N(C(=O)c3ccc(-c4cnn5cccnc45)cc3F)[C@@H]3CCCNC3)c12] |
| 628 | (R)-2-methyl-4-(5-methyl-1,3,4-thiadiazol- 2-yl)-N-(8-methylisoquinolin-1-yl)-N- (piperidin-3-yl)benzamide | 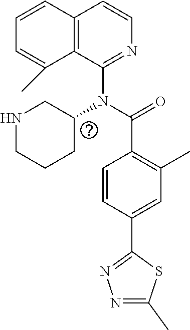 [SMILES: Cc1nnc(-c2ccc(C(=O)N(c3nccc4cccc(C)c34)[C@@H]3CCCNC3)c(C)c2)s1] |
| 629 | 6-(5-methyl-1,3,4-thiadiazol-2-yl)-N-(3- methylthieno[3,2-c]pyridin-4-yl)-N-[(3R)- 3-piperidyl]pyridine-3-carboxamide | 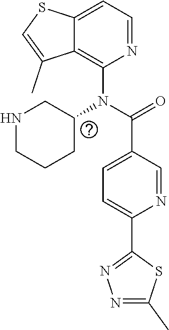 [SMILES: Cc1nnc(-c2ccc(C(=O)N(c3nccc4scc(C)c34)[C@@H]3CCCNC3)cn2)s1] |
| 630 | (R)-4-(4-(methoxymethyl)-1H-1,2,3- triazol-1-yl)-N-(3-methylthieno[3,2- c]pyridin-4-yl)-N-(piperidin-3- yl)benzamide | 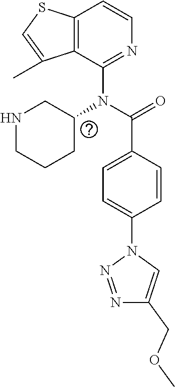 |
| 631 | (R)-N-(6-chloro-8-methylisoquinolin-1- yl)-6-(4-(methoxymethyl)-1H-1,2,3- triazol-1-yl)-N-(piperidin-3- yl)nicotinamide | 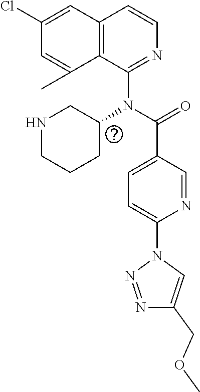 [SMILES: COCc1cn(-c2ccc(C(=O)N(c3nccc4cc(Cl)cc(C)c34)[C@@H]3CCCNC3)cn2)nn1] |
| 632 | (R)-N-(6-chloro-8-methylisoquinolin-1- yl)-5-(4-(methoxymethyl)-1H-1,2,3- triazol-1-yl)-N-(piperidin-3- yl)picolinamide | 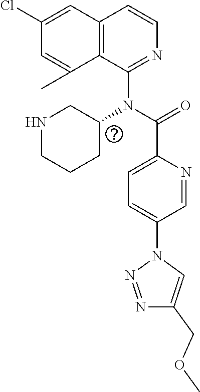 [SMILES: COCc1cn(-c2ccc(C(=O)N(c3nccc4cc(Cl)cc(C)c34)[C@@H]3CCCNC3)nc2)nn1] |
| 633 | (R)-4-(5-(2-methoxyethyl)-1,3,4- thiadiazol-2-yl)-N-(3-methylthieno[3,2- c]pyridin-4-yl)-N-(piperidin-3- yl)benzamide | 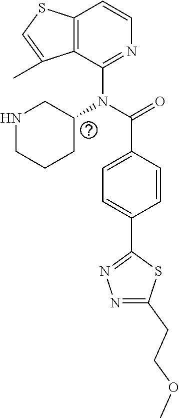 [SMILES: COCCc1nnc(-c2ccc(C(=O)N(c3nccc4scc(C)c34)[C@@H]3CCCNC3)cc2)s1] |
| 634 | (R)-2-fluoro-4-(5-(2-methoxyethyl)-1,3,4- thiadiazol-2-yl)-N-(3-methylthieno[3,2- c]pyridin-4-yl)-N-(piperidin-3- yl)benzamide | 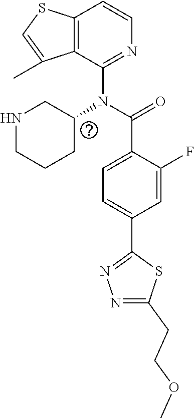 [SMILES: COCCc1nnc(-c2ccc(C(=O)N(c3nccc4scc(C)c34)[C@@H]3CCCNC3)c(F)c2)s1] |
| 635 | (R)-4-(6,7-dihydro-4H-[1,2,3]triazolo[5,1- c][1,4]oxazin-3-yl)-2-fluoro-N-(3- methylthieno[3,2-c]pyridin-4-yl)-N- (piperidin-3-yl)benzamide | 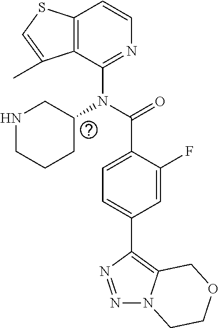 [SMILES: Cc1csc2ccnc(N(C(=O)c3ccc(-c4nnn5c4COCC5)cc3F)[C@@H]3CCCNC3)c12] |
| 636 | (R)-N-(6-chloro-8-methylisoquinolin-1- yl)-2-fluoro-4-(5-(2-methoxyethyl)-1- methyl-1H-1,2,3-triazol-4-yl)-N- (piperidin-3-yl)benzamide | 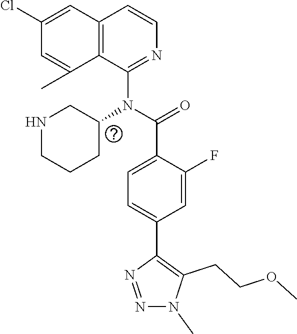 [SMILES: COCCc1c(-c2ccc(C(=O)N(c3nccc4cc(Cl)cc(C)c34)[C@@H]3CCCNC3)c(F)c2)nnn1C] |
| 637 | (R)-N-(6-chloro-8- methylisoquinolin-1-yl)-2-fluoro-4-(5- (methoxymethyl)-1-methyl-1H-1,2,3- triazol-4-yl)-N-(piperidin-3-yl)benzamide | 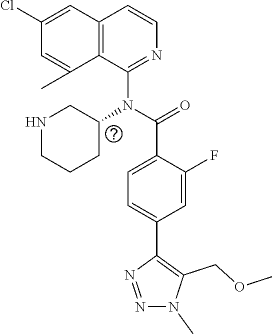 [SMILES: COCc1c(-c2ccc(C(=O)N(c3nccc4cc(Cl)cc(C)c34)[C@@H]3CCCNC3)c(F)c2)nnn1C] |
| 638 | (R)-6-(1-(2-methoxyethyl)-1H-1,2,3- triazol-4-yl)-N-(3-methylthieno[3,2- c]pyridin-4-yl)-N-(piperidin-3- yl)nicotinamide | 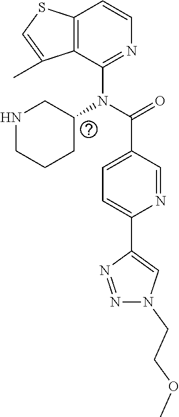 [SMILES: COCCn1cc(-c2ccc(C(=O)N(c3nccc4scc(C)c34)[C@@H]3CCCNC3)cn2)nn1] |
| 639 | (R)-4-(1-(2-methoxyethyl)-1H-1,2,3- triazol-4-yl)-N-(3-methylthieno[3,2- c]pyridin-4-yl)-N-(piperidin-3- yl)benzamide | 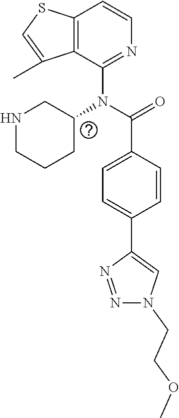 [SMILES: COCCn1cc(-c2ccc(C(=O)N(c3nccc4scc(C)c34)[C@@H]3CCCNC3)cc2)nn1] |
| 640 | (R)-2-fluoro-4-(1-(2-methoxyethyl)-1H- 1,2,3-triazol-4-yl)-N-(3-methylthieno[3,2- c]pyridin-4-yl)-N-(piperidin-3- yl)benzamide | 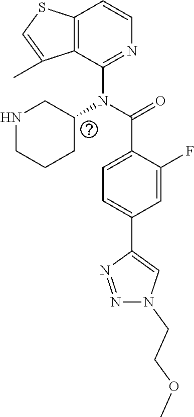 [SMILES: COCCn1cc(-c2ccc(C(=O)N(c3nccc4scc(C)c34)[C@@H]3CCCNC3)c(F)c2)nn1] |
| 641 | (R)-N-(6,8-dimethylisoquinolin-1-yl)-2- fluoro-4-(5-methyl-1,3,4-thiadiazol-2-yl)- N-(piperidin-3-yl)benzamide | 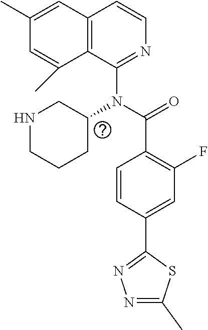 [SMILES: Cc1cc(C)c2c(N(C(=O)c3ccc(-c4nnc(C)s4)cc3F)[C@@H]3CCCNC3)nccc2c1] |
| 642 | (R)-N-(6,8-dimethylisoquinolin-1-yl)-6-(5- methyl-1,3,4-oxadiazol-2-yl)-N-(piperidin- 3-yl)nicotinamide | 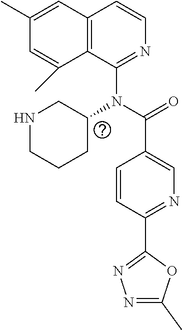 |
| 643 | (R)-N-(6,8-dimethylisoquinolin-1-yl)-2- fluoro-4-(5-methyl-1,3,4-oxadiazol-2-yl)- N-(piperidin-3-yl)benzamide | 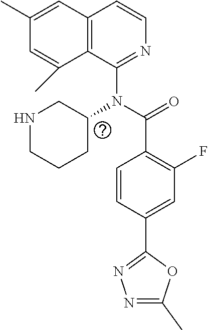 [SMILES: Cc1cc(C)c2c(N(C(=O)c3ccc(-c4nnc(C)o4)cc3F)[C@@H]3CCCNC3)nccc2c1] |
| 644 | 4-(2,2-dimethylmorpholin-4-yl)-2-fluoro- N-(8-methyl-1-isoquinolyl)-N-[(3R)-3- piperidyl] benzamide | 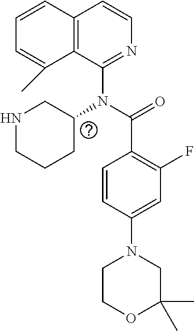 [SMILES: Cc1cccc2ccnc(N(C(=O)c3ccc(N4CCOC(C)(C)C4)cc3F)[C@@H]3CCCNC3)c12] |
| 645 | 4-(2,3-dihydro-1,4-benzoxazin-4-yl)-2- fluoro-N-(8-methyl-1-isoquinolyl)-N- [(3R)-3-piperidyl] benzamide | 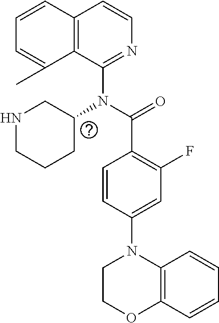 [SMILES: Cc1cccc2ccnc(N(C(=O)c3ccc(N4CCOc5ccccc54)cc3F)[C@@H]3CCCNC3)c12] |
| 646 | 4-(2,3-dihydropyrido[3,2-b][1,4]oxazin-4- yl)-2-fluoro-N-(8-methyl-1-isoquinolyl)-N- [(3R)-3-piperidyl]benzamide | 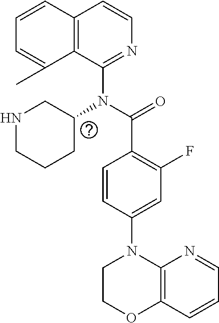 [SMILES: Cc1cccc2ccnc(N(C(=O)c3ccc(N4CCOc5cccnc54)cc3F)[C@@H]3CCCNC3)c12] |
| 647 | N-(6-fluoro-8-methyl-1-isoquinolyl)-6-(5- methyl-1, 3, 4-thiadiazol-2-yl)-N-[(3R)-3- piperidyl] pyridine-3-carboxamide | 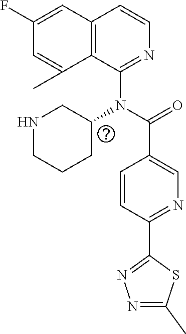 [SMILES: Cc1nnc(-c2ccc(C(=O)N(c3nccc4cc(F)cc(C)c34)[C@@H]3CCCNC3)cn2)s1] |
| 648 | (R)-N-(6,8-dimethylisoquinolin-1-yl)-6-(5- methyl-1,3,4-thiadiazol-2-yl)-N-(piperidin- 3-yl)nicotinamide | 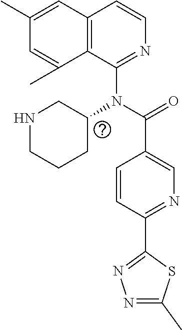 |
| 649 | (R)-4-(5-ethyl-1,3,4-oxadiazol-2-yl)-2- fluoro-N-(3-methylthieno[3,2-c]pyridin-4- yl)-N-(piperidin-3-yl)benzamide | 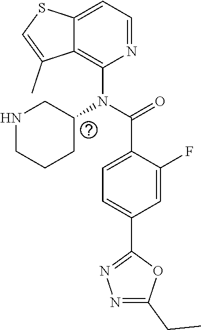 [SMILES: CCc1nnc(-c2ccc(C(=O)N(c3nccc4scc(C)c34)[C@@H]3CCCNC3)c(F)c2)o1] |
| 650 | 2-fluoro-N-(6-fluoro-8-methyl-1- isoquinolyl)-4-(5-hydroxy-1,3,4-thiadiazol- 2-yl)-N-[(3R)-3-piperidyl]benzamide | 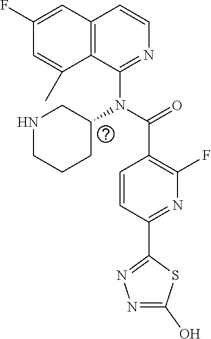 [SMILES: Cc1cc(F)cc2ccnc(N(C(=O)c3ccc(-c4nnc(O)s4)nc3F)[C@@H]3CCCNC3)c12] |
| 651 | (R)-2-fluoro-4-(5-methyl-1,3,4-thiadiazol- 2-yl)-N-(piperidin-3-yl)-N-(3-(prop-1-en- 2-yl)thieno[3,2-c]pyridin-4-yl)benzamide | 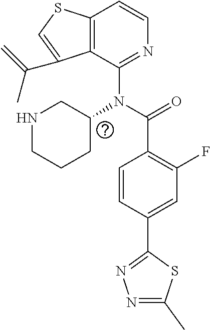 [SMILES: C=C(C)c1csc2ccnc(N(C(=O)c3ccc(-c4nnc(C)s4)cc3F)[C@@H]3CCCNC3)c12] |
| 652 | (R)-N-(3-ethynylthieno[3,2-c]pyridin-4- yl)-2-fluoro-4-(5-methyl-1,3,4-thiadiazol- 2-yl)-N-(piperidin-3-yl)benzamide | 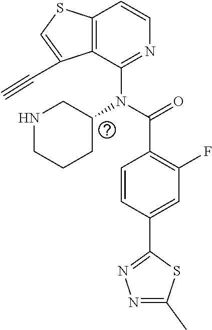 [SMILES: C#Cc1csc2ccnc(N(C(=O)c3ccc(-c4nnc(C)s4)cc3F)[C@@H]3CCCNC3)c12] |
| 653 | (R)-N-(3-chlorothieno[3,2-c]pyridin-4-yl)- 2-fluoro-4-(5-methyl-1,3,4-thiadiazol-2- yl)-N-(piperidin-3-yl)benzamide | 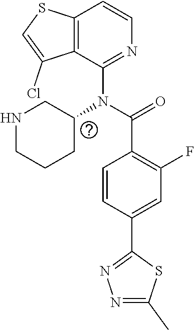 [SMILES: Cc1nnc(-c2ccc(C(=O)N(c3nccc4scc(Cl)c34)[C@@H]3CCCNC3)c(F)c2)s1] |
| 654 | (R)-N-(6-chloro-8-methylisoquinolin-1- yl)-4-(5-ethyl-1,3,4-oxadiazol-2-yl)-2- fluoro-N-(piperidin-3-yl)benzamide | 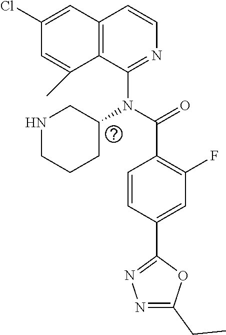 [SMILES: CCc1nnc(-c2ccc(C(=O)N(c3nccc4cc(Cl)cc(C)c34)[C@@H]3CCCNC3)c(F)c2)o1] |
| 655 | (R)-2-fluoro-N-(5-fluoro-8- methylisoquinolin-1-yl)-4-(4-methyl-1H- 1,2,3-triazol-1-yl)-N-(piperidin-3- yl)benzamide | 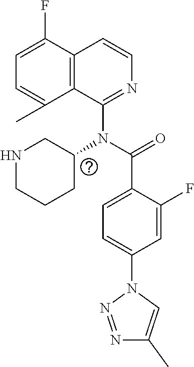 [SMILES: Cc1cn(-c2ccc(C(=O)N(c3nccc4c(F)ccc(C)c34)[C@@H]3CCCNC3)c(F)c2)nn1] |
| 656 | (R)-N-(5-fluoro-8-methylisoquinolin-1-yl)- 4-(1-methyl-1H-1,2,3-triazol-4-yl)-N- (piperidin-3-yl)benzamide | 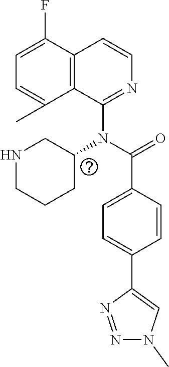 [SMILES: Cc1ccc(F)c2ccnc(N(C(=O)c3ccc(-c4cn(C)nn4)cc3)[C@@H]3CCCNC3)c12] |
| 657 | (R)-4-(5-ethyl-1,3,4-thiadiazol-2-yl)-2- fluoro-N-(5-fluoro-8-methylisoquinolin-1- yl)-N-(piperidin-3-yl)benzamide | 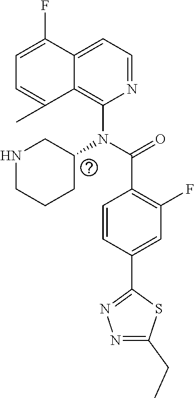 [SMILES: CCc1nnc(-c2ccc(C(=O)N(c3nccc4c(F)ccc(C)c34)[C@@H]3CCCNC3)c(F)c2)s1] |
| 658 | (R)-N-(6-fluoro-8-methylisoquinolin-1-yl)- 4-(3-methylisoxazol-5-yl)-N-(piperidin-3- yl)piperidine-1-carboxamide | 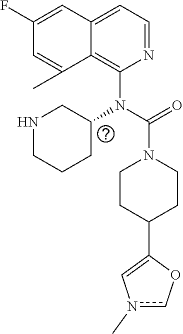 |
| 659 | (R)-N-(5-fluoro-8-methylisoquinolin-1-yl)- 4-(3-methylisoxazol-5-yl)-N-(piperidin-3- yl)piperidine-1-carboxamide | 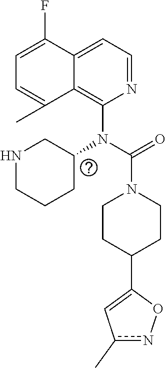 |
| 660 | (R)-2-fluoro-N-(5-fluoro-8- methylisoquinolin-1-yl)-4-(5-methyl-1,3,4- oxadiazol-2-yl)-N-(piperidin-3- yl)benzamide | 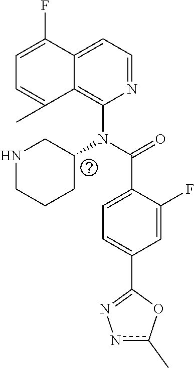 |
| 661 | (R)-2-fluoro-N-(5-fluoro-8- methylisoquinolin-1-yl)-N-(piperidin-3- yl)-4-(pyrimidin-2-ylamino)benzamide | 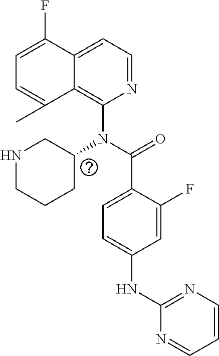 [SMILES: Cc1ccc(F)c2ccnc(N(C(=O)c3ccc(Nc4ncccn4)cc3F)[C@@H]3CCCNC3)c12] |
| 662 | (R)-2-fluoro-N-(8-methylisoquinolin-1-yl)- N-(piperidin-3-yl)-4-((4-(piperidin-4- yl)pyrimidin-2-yl)amino)benzamide | 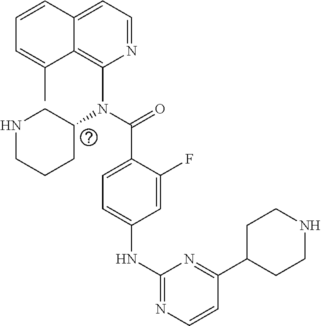 [SMILES: Cc1cccc2ccnc(N(C(=O)c3ccc(Nc4nccc(C5CCNCC5)n4)cc3F)[C@@H]3CCCNC3)c12] |
| 663 | (R)-2-fluoro-N-(8-methylisoquinolin-1-yl)- N-(piperidin-3-yl)-4-((4-(tetrahydro-2H- pyran-4-yl)pyrimidin-2- yl)amino)benzamide | 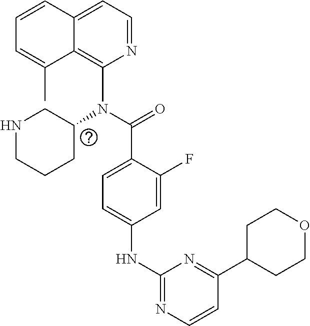 [SMILES: Cc1cccc2ccnc(N(C(=O)c3ccc(Nc4nccc(C5CCOCC5)n4)cc3F)[C@@H]3CCCNC3)c12] |
| 664 | (R)-N-(3-chloro-1-methyl-1H-pyrrolo[2,3- c]pyridin-7-yl)-2-fluoro-4-(5-methyl-1,3,4- thiadiazol-2-yl)-N-(piperidin-3- yl)benzamide | 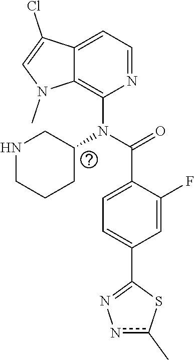 |
| 665 | (R)-2-fluoro-N-(8-methylisoquinolin-1-yl)- N-(piperidin-3-yl)-4-((4-(1,2,3,6- tetrahydropyridin-4-yl)pyrimidin-2- yl)amino)benzamide | 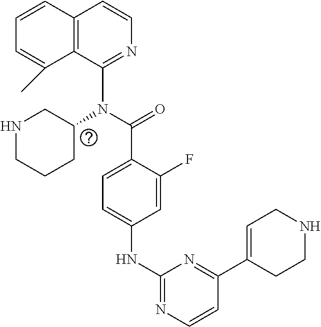 [SMILES: Cc1cccc2ccnc(N(C(=O)c3ccc(Nc4nccc(C5=CCNCC5)n4)cc3F)[C@@H]3CCCNC3)c12] |
| 667 | (R)-4-(5-ethyl-1,3,4-thiadiazol-2-yl)-2- fluoro-N-(2-methylthiazolo[4,5-c]pyridin- 4-yl)-N-(piperidin-3-yl)benzamide | 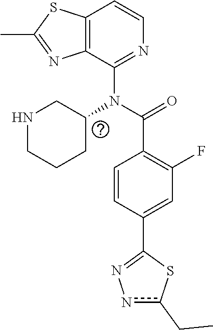 |
| 668 | N-(3-chlorothieno[3,2-c]pyridin-4-yl)-5-(5- ethyl-1,3,4-thiadiazol-2-yl)-N-[(3R)-3- piperidyl]pyridine-2-carboxamide | 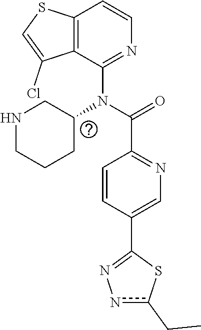 |
| 669 | 5-(5-ethyl-1,3,4-thiadiazol-2-yl)-N-(3- methylthieno[2,3-c]pyridin-7-yl)-N-[(3R)- 3-piperidyl]pyridine-2-carboxamide | 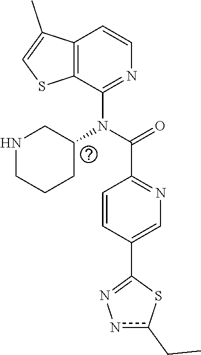 |
| 670 | (R)-5-(5-ethyl-1,3,4-thiadiazol-2-yl)-N-(3- methylthieno[3,2-c]pyridin-4-yl)-N- (piperidin-3-yl)picolinamide | 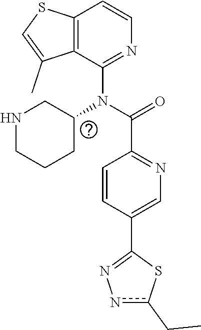 |
| 671 | (R)-N-(6,8-dimethylisoquinolin-1-yl)-5-(5- ethyl-1,3,4-thiadiazol-2-yl)-N-(piperidin-3- yl)picolinamide | 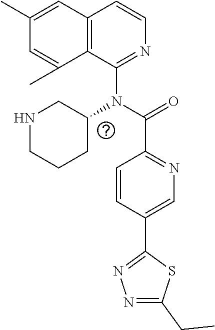 [SMILES: CCc1nnc(-c2ccc(C(=O)N(c3nccc4cc(C)cc(C)c34)[C@@H]3CCCNC3)nc2)s1] |
| 672 | (R)-5-(5-ethyl-1,3,4-thiadiazol-2-yl)-N-(6- fluoro-8-methylisoquinolin-1-yl)-N- (piperidin-3-yl)picolinamide | 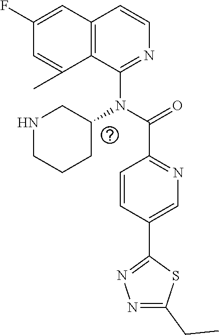 [SMILES: CCc1nnc(-c2ccc(C(=O)N(c3nccc4cc(F)cc(C)c34)[C@@H]3CCCNC3)nc2)s1] |
| 673 | 5-(5-ethyl-1,3,4-thiadiazol-2-yl)-N-(8- methyl-1-isoquinolyl)-N-[(3R)-3- piperidyl]pyridine-2-carboxamide | 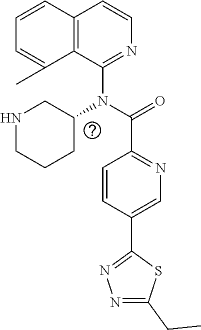 [SMILES: CCc1nnc(-c2ccc(C(=O)N(c3nccc4cccc(C)c34)[C@@H]3CCCNC3)nc2)s1] |
| 674 | (R)-N-(3-ethynylthieno[3,2-c]pyridin-4- yl)-2-fluoro-4-(1-methyl-1H-1,2,3-triazol- 4-yl)-N-(piperidin-3-yl)benzamide | 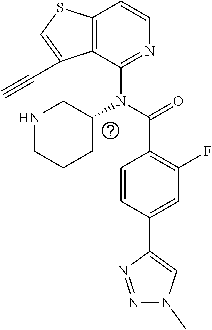 [SMILES: C#Cc1csc2ccnc(N(C(=O)c3ccc(-c4cn(C)nn4)cc3F)[C@@H]3CCCNC3)c12] |
| 675 | (R)-2-fluoro-4-(1-methyl-1H-1,2,3-triazol- 4-yl)-N-(piperidin-3-yl)-N-(3-(prop-1-en- 2-yl)thieno[2,3-c]pyridin-7-yl)benzamide | 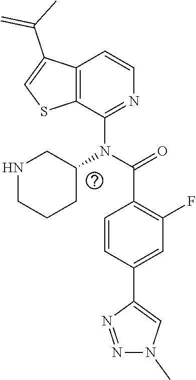 |
| 676 | (R)-N-(3-ethynylthieno[2,3-c]pyridin-7- yl)-2-fluoro-4-(1-methyl-1H-1,2,3-triazol- 4-yl)-N-(piperidin-3-yl)benzamide | 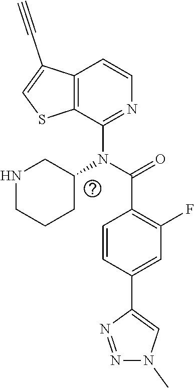 |
| 677 | (R)-2-fluoro-4-(1-methyl-1H-1,2,3-triazol- 4-yl)-N-(3-methylthieno[2,3-c]pyridin-7- yl)-N-(piperidin-3-yl)benzamide | 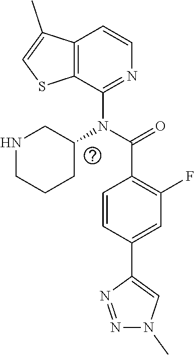 |
| 678 | (R)-N-(3-chlorothieno[2,3-c]pyridin-7-yl)- 2-fluoro-4-(1-methyl-1H-1,2,3-triazol-4- yl)-N-(piperidin-3-yl)benzamide | 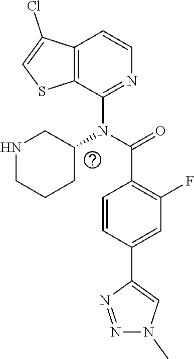 |
| 679 | (R)-2-fluoro-4-((4-(4- hydroxycyclohexyl)pyrimidin-2-yl)amino)- N-(8-methylisoquinolin-1-yl)-N-(piperidin- 3-yl)benzamide | 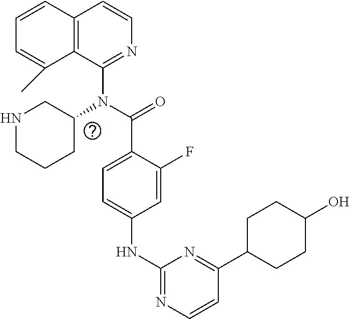 [SMILES: Cc1cccc2ccnc(N(C(=O)c3ccc(Nc4nccc(C5CCC(O)CC5)n4)cc3F)[C@@H]3CCCNC3)c12] |
| 680 | (R)-2-fluoro-4-((4-(3-hydroxyazetidin-1- yl)pyrimidin-2-yl)amino)-N-(8- methylisoquinolin-1-yl)-N-(piperidin-3- yl)benzamide | 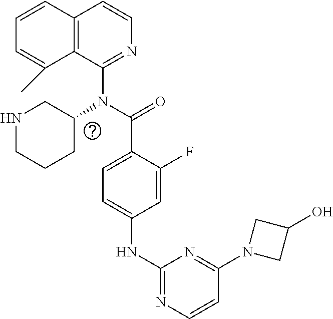 [SMILES: Cc1cccc2ccnc(N(C(=O)c3ccc(Nc4nccc(N5CC(O)C5)n4)cc3F)[C@@H]3CCCNC3)c12] |
| 681 | 2-fluoro-4-((4-(4-hydroxycyclohex-1-en-1- yl)pyrimidin-2-yl)amino)-N-(8- methylisoquinolin-1-yl)-N-((R)-piperidin- 3-yl)benzamide | 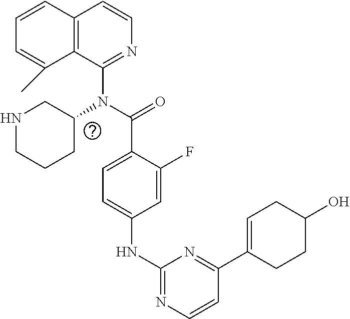 [SMILES: Cc1cccc2ccnc(N(C(=O)c3ccc(Nc4nccc(C5=CCC(O)CC5)n4)cc3F)[C@@H]3CCCNC3)c12] |
| 682 | (R)-2-fluoro-4-((4-(1-methyl-1,2,3,6- tetrahydropyridin-4-yl)pyrimidin-2- yl)amino)-N-(8-methylisoquinolin-1-yl)-N- (piperidin-3-yl)benzamide | 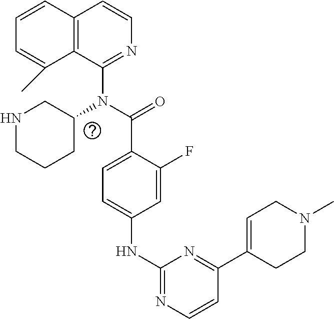 [SMILES: Cc1cccc2ccnc(N(C(=O)c3ccc(Nc4nccc(C5=CCN(C)CC5)n4)cc3F)[C@@H]3CCCNC3)c12] |
| 683 | (R)-2-fluoro-N-(8-methylisoquinolin-1-yl)- N-(piperidin-3-yl)-4-((4-(pyridin-4- ylamino)pyrimidin-2-yl)amino)benzamide | 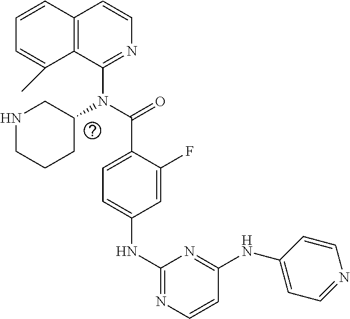 [SMILES: Cc1cccc2ccnc(N(C(=O)c3ccc(Nc4nccc(Nc5ccncc5)n4)cc3F)[C@@H]3CCCNC3)c12] |
| 684 | 2-fluoro-4-((4-((R)-3-hydroxypyrrolidin-1- yl)pyrimidin-2-yl)amino)-N-(8- methylisoquinolin-1-yl)-N-((R)-piperidin- 3-yl)benzamide | 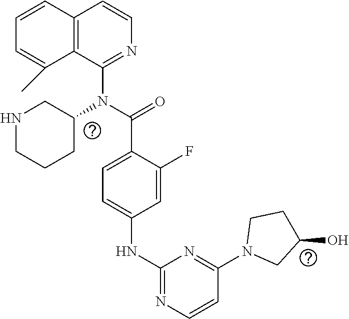 [SMILES: Cc1cccc2ccnc(N(C(=O)c3ccc(Nc4nccc(N5CC[C@@H](O)C5)n4)cc3F)[C@@H]3CCCNC3)c12] |
| 685 | (R)-4-((4-(3,6-dihydro-2H-pyran-4- yl)pyrimidin-2-yl)amino)-2-fluoro-N-(8- methylisoquinolin-1-yl)-N-(piperidin-3- yl)benzamide | 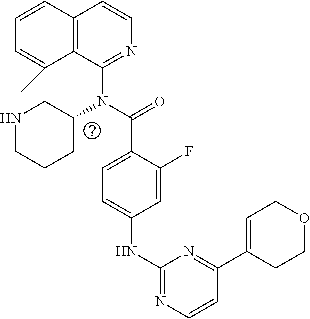 [SMILES: Cc1cccc2ccnc(N(C(=O)c3ccc(Nc4nccc(C5=CCOCC5)n4)cc3F)[C@@H]3CCCNC3)c12] |
| 686 | (R)-2-fluoro-N-(8-methylisoquinolin-1-yl)- 4-((4-(piperazin-1-ylmethyl)pyrimidin-2- yl)amino)-N-(piperidin-3-yl)benzamide | 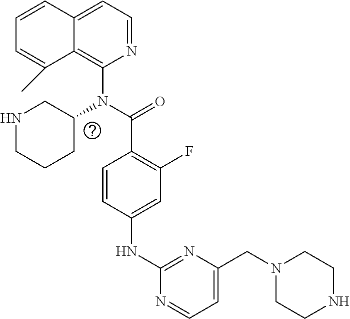 [SMILES: Cc1cccc2ccnc(N(C(=O)c3ccc(Nc4nccc(CN5CCNCC5)n4)cc3F)[C@@H]3CCCNC3)c12] |
| 687 | (R)-2-fluoro-N-(8-methylisoquinolin-1-yl)- 4-((4-(piperidin-1-ylmethyl)pyrimidin-2- yl)amino)-N-(piperidin-3-yl)benzamide | 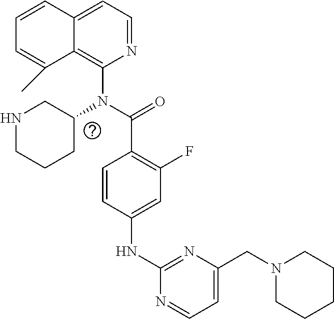 [SMILES: Cc1cccc2ccnc(N(C(=O)c3ccc(Nc4nccc(CN5CCCCC5)n4)cc3F)[C@@H]3CCCNC3)c12] |
| 688 | (R)-1-(2-fluoro-4-(1-methyl-1H-1,2,3- triazol-4-yl)-N-(piperidin-3- yl)benzamido)-N-methylisoquinoline-6- carboxamide | 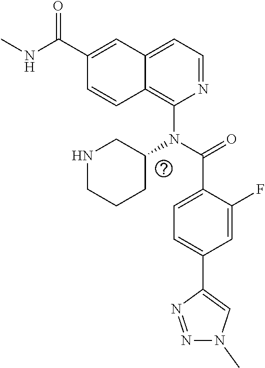 |
| 689 | (R)-4-(4-methyl-1H-1,2,3-triazol-1-yl)-N- (3-methylthieno[3,2-c]pyridin-4-yl)-N- (piperidin-3-yl)benzamide | 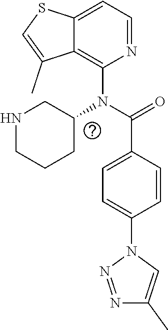 |
| 690 | (R)-2-fluoro-4-(4-methyl-1H-1,2,3-triazol- 1-yl)-N-(3-methylthieno[3,2-c]pyridin-4- yl)-N-(piperidin-3-yl)benzamide | 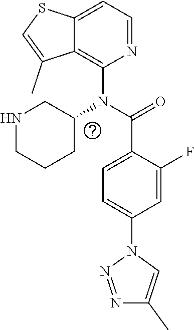 |
| 691 | (R)-N-(6-chloro-8-methylisoquinolin-1- yl)-2-fluoro-4-(4-methyl-1H-1,2,3-triazol- 1-yl)-N-(piperidin-3-yl)benzamide | 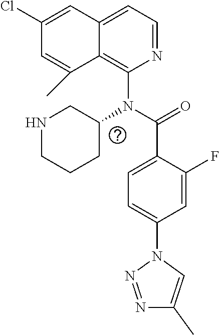 [SMILES: Cc1cn(-c2ccc(C(=O)N(c3nccc4cc(Cl)cc(C)c34)[C@@H]3CCCNC3)c(F)c2)nn1] |
| 692 | (R)-2-fluoro-4-(1-methyl-1H-1,2,3-triazol- 4-yl)-N-(piperidin-3-yl)-N-(6-(prop-1-en- 2-yl)isoquinolin-1-yl)benzamide | 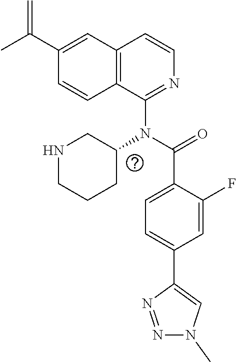 [SMILES: C=C(C)c1ccc2c(N(C(=O)c3ccc(-c4cn(C)nn4)cc3F)[C@@H]3CCCNC3)nccc2c1] |
| 693 | (R)-2-fluoro-N-(8-methylisoquinolin-1-yl)- N-(piperidin-3-yl)-4-((4-((thiazol-2- ylmethyl)amino)pyrimidin-2- yl)amino)benzamide | 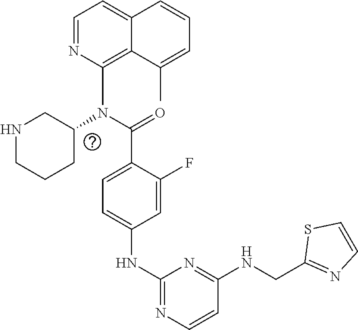 [SMILES: Cc1cccc2ccnc(N(C(=O)c3ccc(Nc4nccc(NCc5nccs5)n4)cc3F)[C@@H]3CCCNC3)c12] |
| 694 | (R)-2-fluoro-4-((4-(((1-methyl-1H-pyrazol- 4-yl)methyl)amino)pyrimidin-2-yl)amino)- N-(8-methylisoquinolin-1-yl)-N-(piperidin- 3-yl)benzamide | 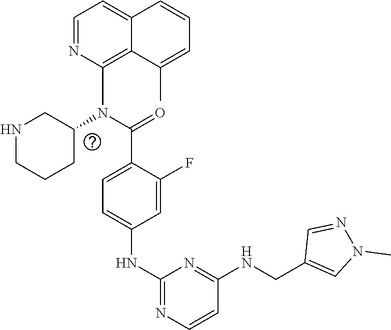 [SMILES: Cc1cccc2ccnc(N(C(=O)c3ccc(Nc4nccc(NCc5cnn(C)c5)n4)cc3F)[C@@H]3CCCNC3)c12] |
| 695 | (R)-2-fluoro-N-(8-methylisoquinolin-1-yl)- N-(piperidin-3-yl)-4-((4-((pyridin-2- ylmethyl)amino)pyrimidin-2- yl)amino)benzamide | 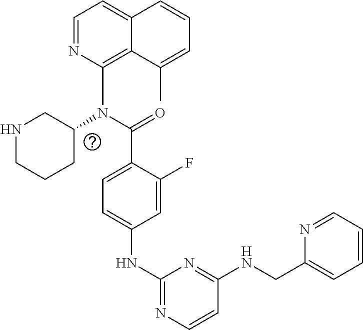 [SMILES: Cc1cccc2ccnc(N(C(=O)c3ccc(Nc4nccc(NCc5ccccn5)n4)cc3F)[C@@H]3CCCNC3)c12] |
| 696 | (R)-2-fluoro-N-(8-methylisoquinolin-1-yl)- N-(piperidin-3-yl)-4-((4-((pyridin-3- ylmethyl)amino)pyrimidin-2- yl)amino)benzamide | 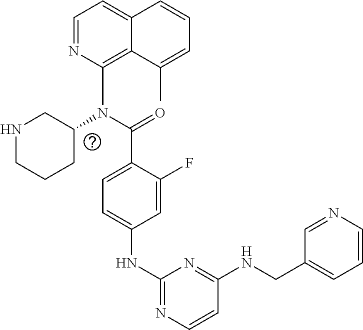 [SMILES: Cc1cccc2ccnc(N(C(=O)c3ccc(Nc4nccc(NCc5cccnc5)n4)cc3F)[C@@H]3CCCNC3)c12] |
| 697 | (R)-2-fluoro-N-(8-methylisoquinolin-1-yl)- N-(piperidin-3-yl)-4-((4-((pyridin-4- ylmethyl)amino)pyrimidin-2- yl)amino)benzamide | 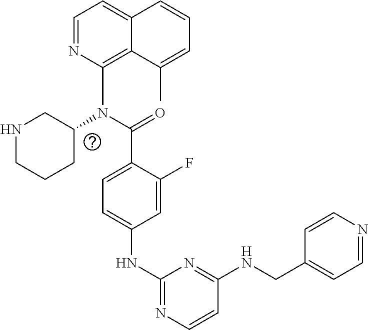 [SMILES: Cc1cccc2ccnc(N(C(=O)c3ccc(Nc4nccc(NCc5ccncc5)n4)cc3F)[C@@H]3CCCNC3)c12] |
| 698 | (R)-4-(5-ethyl-1,3,4-thiadiazol-2-yl)-2- fluoro-N-(3-methylthieno[3,2-c]pyridin-4- yl)-N-(piperidin-3-yl)benzamide | 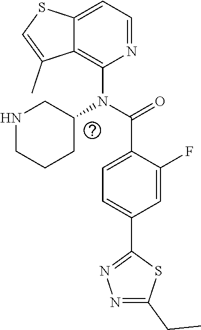 [SMILES: CCc1nnc(-c2ccc(C(=O)N(c3nccc4scc(C)c34)[C@@H]3CCCNC3)c(F)c2)s1] |
| 699 | (R)-2-fluoro-4-(5-methyl-1,3,4-oxadiazol- 2-yl)-N-(3-methylthieno[3,2-c]pyridin-4- yl)-N-(piperidin-3-yl)benzamide | 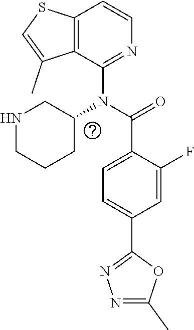 [SMILES: Cc1nnc(-c2ccc(C(=O)N(c3nccc4scc(C)c34)[C@@H]3CCCNC3)c(F)c2)o1] |
| 700 | (R)-N-(6-chloro-8-methylisoquinolin-1- yl)-4-(5-ethyl-1,3,4-thiadiazol-2-yl)-2- fluoro-N-(piperidin-3-yl)benzamide | 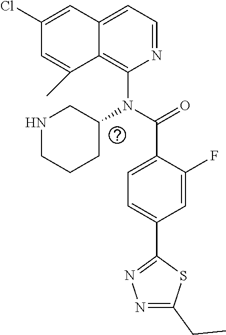 [SMILES: CCc1nnc(-c2ccc(C(=O)N(c3nccc4cc(Cl)cc(C)c34)[C@@H]3CCCNC3)c(F)c2)s1] |
| 701 | (R)-N-(6,8-dimethylisoquinolin-1-yl)-5-(5- methyl-1,3,4-thiadiazol-2-yl)-N-(piperidin- 3-yl)picolinamide | 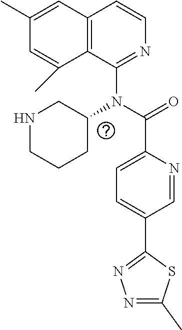 |
| 702 | (R)-N-(6,8-dimethylisoquinolin-1-yl)-2- fluoro-4-(1-methyl-1H-1,2,3-triazol-4-yl)- N-(piperidin-3-yl)benzamide | 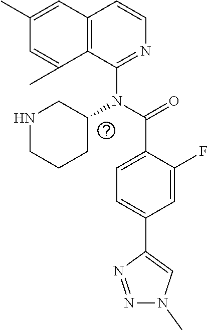 [SMILES: Cc1cc(C)c2c(N(C(=O)c3ccc(-c4cn(C)nn4)cc3F)[C@@H]3CCCNC3)nccc2c1] |
| 703 | (R)-N-(6,8-dimethylisoquinolin-1-yl)-5-(1- methyl-1H-1,2,3-triazol-4-yl)-N- (piperidin-3-yl)picolinamide | 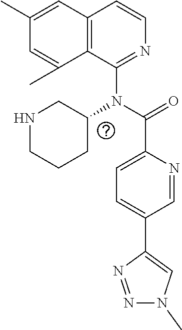 |
| 704 | (R)-4-(1-ethyl-1H-1,2,3-triazol-4-yl)-2- fluoro-N-(3-methylthieno[3,2-c]pyridin-4- yl)-N-(piperidin-3-yl)benzamide | 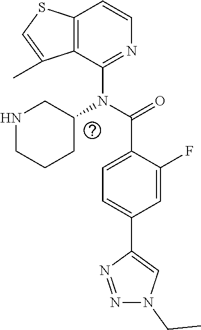 [SMILES: CCn1cc(-c2ccc(C(=O)N(c3nccc4scc(C)c34)[C@@H]3CCCNC3)c(F)c2)nn1] |
| 705 | (R)-2-methyl-4-(1-methyl-1H-1,2,3- triazol-4-yl)-N-(8-methylisoquinolin-1-yl)- N-(piperidin-3-yl)benzamide | 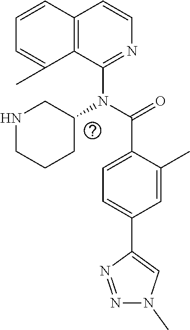 [SMILES: Cc1cc(-c2cn(C)nn2)ccc1C(=O)N(c1nccc2cccc(C)c12)[C@@H]1CCCNC1] |
| 706 | (R)-N-(6-chloro-8-methylisoquinolin-1- yl)-3-methoxy-4-(5-methyl-1,3,4- thiadiazol-2-yl)-N-(piperidin-3- yl)benzamide | 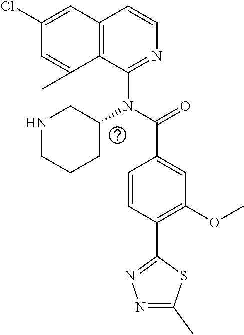 [SMILES: COc1cc(C(=O)N(c2nccc3cc(Cl)cc(C)c23)[C@@H]2CCCNC2)ccc1-c1nnc(C)s1] |
| 707 | (R)-N-(6-chloro-8-methylisoquinolin-1- yl)-3-methyl-4-(5-methyl-1,3,4-thiadiazol- 2-yl)-N-(piperidin-3-yl)benzamide | 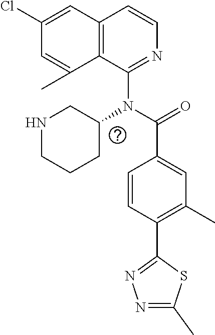 [SMILES: Cc1nnc(-c2ccc(C(=O)N(c3nccc4cc(Cl)cc(C)c34)[C@@H]3CCCNC3)cc2C)s1] |
| 708 | (R)-N-(6-chloro-8-methylisoquinolin-1- yl)-3-fluoro-4-(5-methyl-1,3,4-thiadiazol- 2-yl)-N-(piperidin-3-yl)benzamide | 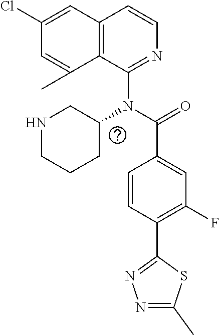 [SMILES: Cc1nnc(-c2ccc(C(=O)N(c3nccc4cc(Cl)cc(C)c34)[C@@H]3CCCNC3)cc2F)s1] |
| 709 | (R)-N-(6-chloro-8-methylisoquinolin-1- yl)-6-(5-methyl-1,3,4-thiadiazol-2-yl)-N- (piperidin-3-yl)nicotinamide | 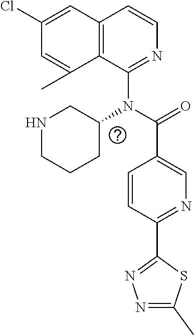 [SMILES: Cc1nnc(-c2ccc(C(=O)N(c3nccc4cc(Cl)cc(C)c34)[C@@H]3CCCNC3)cn2)s1] |
| 710 | (R)-2-fluoro-N-(6-fluoro-8- methylisoquinolin-1-yl)-4-(5-methoxy- 1,3,4-thiadiazol-2-yl)-N-(piperidin-3- yl)benzamide | 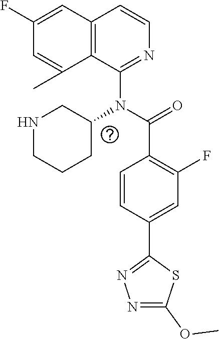 [SMILES: COc1nnc(-c2ccc(C(=O)N(c3nccc4cc(F)cc(C)c34)[C@@H]3CCCNC3)c(F)c2)s1] |
| 711 | N-(6-chloro-8-methyl-1-isoquinolyl)-4-(1- ethyltriazol-4-yl)-2-fluoro-N-[(3R)-3- piperidyl] benzamide | 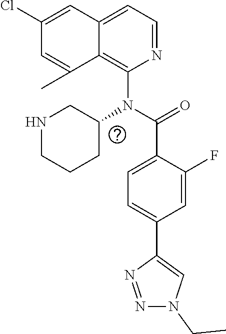 [SMILES: CCn1cc(-c2ccc(C(=O)N(c3nccc4cc(Cl)cc(C)c34)[C@@H]3CCCNC3)c(F)c2)nn1] |
| 712 | (R)-4-(1-ethyl-1H-1,2,3-triazol-4-yl)-2- fluoro-N-(6-fluoro-8-methylisoquinolin-1- yl)-N-(piperidin-3-yl)benzamide | 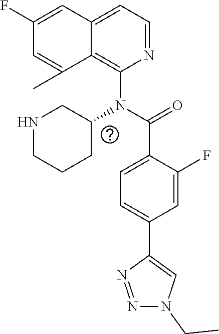 [SMILES: CCn1cc(-c2ccc(C(=O)N(c3nccc4cc(F)cc(C)c34)[C@@H]3CCCNC3)c(F)c2)nn1] |
| 713 | (R)-4-((4-(4-ethylpiperazin-1-yl)pyrimidin- 2-yl)amino)-2-fluoro-N-(8- methylisoquinolin-1-yl)-N-(piperidin-3- yl)benzamide | 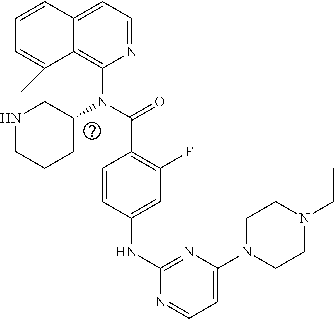 [SMILES: CCN1CCN(c2ccnc(Nc3ccc(C(=O)N(c4nccc5cccc(C)c45)[C@@H]4CCCNC4)c(F)c3)n2)CC1] |
| 714 | (R)-4-((4-(4-(dimethylamino)piperidin-1- yl)pyrimidin-2-yl)amino)-2-fluoro-N-(8- methylisoquinolin-1-yl)-N-(piperidin-3- yl)benzamide) | 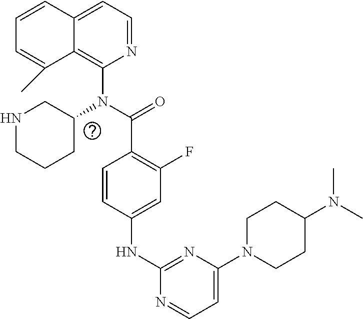 [SMILES: Cc1cccc2ccnc(N(C(=O)c3ccc(Nc4nccc(N5CCC(N(C)C)CC5)n4)cc3F)[C@@H]3CCCNC3)c12] |
| 715 | (R)-2-fluoro-N-(8-methylisoquinolin-1-yl)- N-(piperidin-3-yl)-4-((4-(4-(pyrrolidin-1- yl)piperidin-1-yl)pyrimidin-2- yl)amino)benzamide | 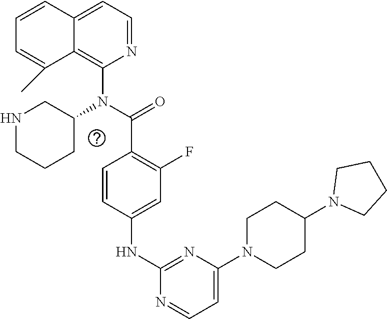 [SMILES: Cc1cccc2ccnc(N(C(=O)c3ccc(Nc4nccc(N5CCC(N6CCCC6)CC5)n4)cc3F)[C@@H]3CCCNC3)c12] |
| 716 | (R)-2-fluoro-4-((4-(4-methoxypiperidin-1- yl)pyrimidin-2-yl)amino)-N-(8- methylisoquinolin-1-yl)-N-(piperidin-3- yl)benzamide | 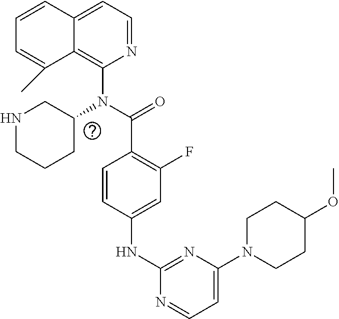 [SMILES: COC1CCN(c2ccnc(Nc3ccc(C(=O)N(c4nccc5cccc(C)c45)[C@@H]4CCCNC4)c(F)c3)n2)CC1] |
| 717 | (R)-2-fluoro-4-((4-(4-hydroxypiperidin-1- yl)pyrimidin-2-yl)amino)-N-(8- methylisoquinolin-1-yl)-N-(piperidin-3- yl)benzamide | 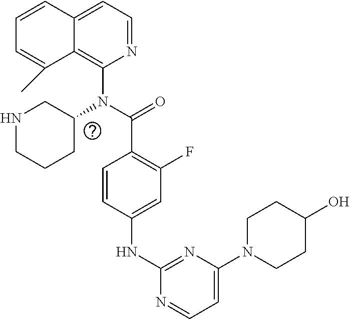 [SMILES: Cc1cccc2ccnc(N(C(=O)c3ccc(Nc4nccc(N5CCC(O)CC5)n4)cc3F)[C@@H]3CCCNC3)c12] |
| 718 | (R)-2-fluoro-N-(8-methylisoquinolin-1-yl)- 4-((4-(piperazin-1-yl)pyrimidin-2- yl)amino)-N-(piperidin-3-yl)benzamide | 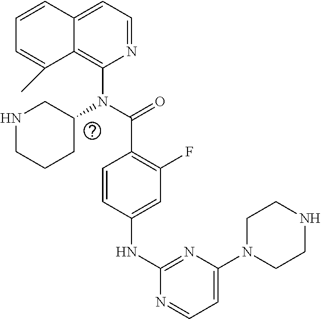 [SMILES: Cc1cccc2ccnc(N(C(=O)c3ccc(Nc4nccc(N5CCNCC5)n4)cc3F)[C@@H]3CCCNC3)c12] |
| 719 | (R)-2-fluoro-N-(8-methylisoquinolin-1-yl)- 4-((4-(4-oxopiperidin-1-yl)pyrimidin-2- yl)amino)-N-(piperidin-3-yl)benzamide | 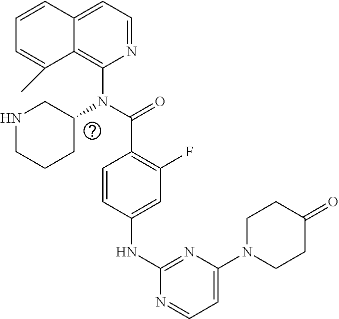 [SMILES: Cc1cccc2ccnc(N(C(=O)c3ccc(Nc4nccc(N5CCC(=O)CC5)n4)cc3F)[C@@H]3CCCNC3)c12] |
| 720 | (R)-4-((4-(6-aminopyridin-3-yl)pyrimidin- 2-yl)amino)-2-fluoro-N-(8- methylisoquinolin-1-yl)-N-(piperidin-3- yl)benzamide | 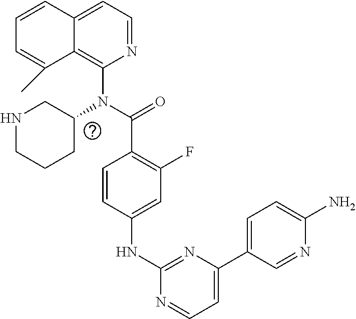 [SMILES: Cc1cccc2ccnc(N(C(=O)c3ccc(Nc4nccc(-c5ccc(N)nc5)n4)cc3F)[C@@H]3CCCNC3)c12] |
| 721 | (R)-2-fluoro-4-((4-(4- hydroxyphenyl)pyrimidin-2-yl)amino)-N- (8-methylisoquinolin-1-yl)-N-(piperidin-3- yl)benzamide | 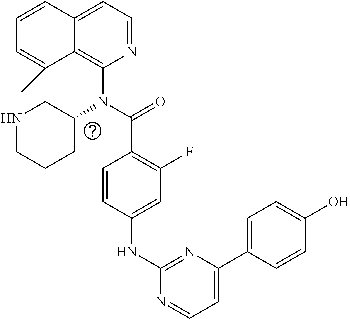 [SMILES: Cc1cccc2ccnc(N(C(=O)c3ccc(Nc4nccc(-c5ccc(O)cc5)n4)cc3F)[C@@H]3CCCNC3)c12] |
| 722 | (R)-4-((4-(1H-1,2,4-triazol-1-yl)pyrimidin- 2-yl)amino)-2-fluoro-N-(8- methylisoquinolin-1-yl)-N-(piperidin-3- yl)benzamide | 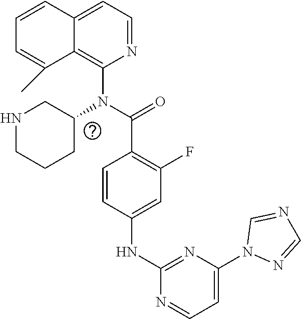 [SMILES: Cc1cccc2ccnc(N(C(=O)c3ccc(Nc4nccc(-n5cncn5)n4)cc3F)[C@@H]3CCCNC3)c12] |
| 723 | (R)-4-((4-(1H-pyrazol-1-yl)pyrimidin-2- yl)amino)-2-fluoro-N-(8- methylisoquinolin-1-yl)-N-(piperidin-3- yl)benzamide | 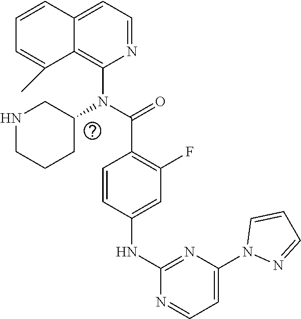 [SMILES: Cc1cccc2ccnc(N(C(=O)c3ccc(Nc4nccc(-n5cccn5)n4)cc3F)[C@@H]3CCCNC3)c12] |
| 724 | (R)-4-((4-(1H-imidazol-1-yl)pyrimidin-2- yl)amino)-2-fluoro-N-(8- methylisoquinolin-1-yl)-N-(piperidin-3- yl)benzamide | 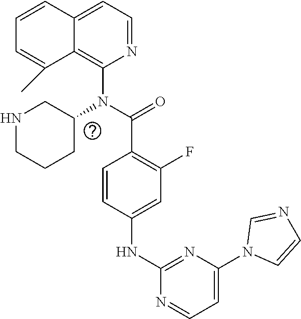 [SMILES: Cc1cccc2ccnc(N(C(=O)c3ccc(Nc4nccc(-n5ccnc5)n4)cc3F)[C@@H]3CCCNC3)c12] |
| 725 | (R)-N-(6-fluoro-8-methylisoquinolin-1-yl)- 4-(5-methyl-1,3,4-thiadiazol-2-yl)-N- (piperidin-3-yl)benzamide | 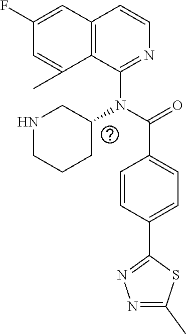 [SMILES: Cc1nnc(-c2ccc(C(=O)N(c3nccc4cc(F)cc(C)c34)[C@@H]3CCCNC3)cc2)s1] |
| 726 | (R)-2-fluoro-4-((3-(methylamino)pyridin- 2-yl)amino)-N-(8-methylisoquinolin-1-yl)- N-(piperidin-3-yl)benzamide | 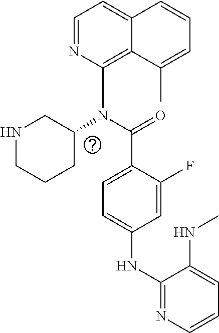 [SMILES: CNc1cccnc1Nc1ccc(C(=O)N(c2nccc3cccc(C)c23)[C@@H]2CCCNC2)c(F)c1] |
| 727 | (R)-2-fluoro-N-(8-methylisoquinolin-1-yl)- N-(piperidin-3-yl)-4-((4-(pyridin-3- yl)pyrimidin-2-yl)amino)benzamide | 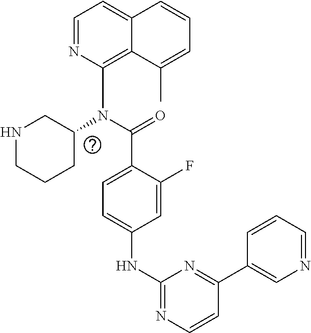 [SMILES: Cc1cccc2ccnc(N(C(=O)c3ccc(Nc4nccc(-c5cccnc5)n4)cc3F)[C@@H]3CCCNC3)c12] |
| 728 | (R)-2-fluoro-N-(8-methylisoquinolin-1-yl)- N-(piperidin-3-yl)-4-((4-(pyridin-2- yl)pyrimidin-2-yl)amino)benzamide | 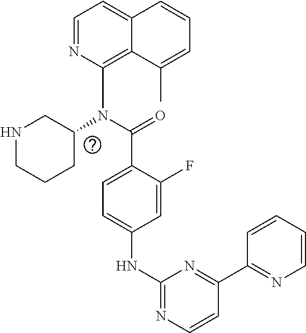 [SMILES: Cc1cccc2ccnc(N(C(=O)c3ccc(Nc4nccc(-c5ccccn5)n4)cc3F)[C@@H]3CCCNC3)c12] |
| 729 | (R)-N-(6-fluoro-8-methylisoquinolin-1-yl)- 5-(1-(2-methoxyethyl)-3-methyl-1H- pyrazol-5-yl)-N-(piperidin-3- yl)picolinamide | 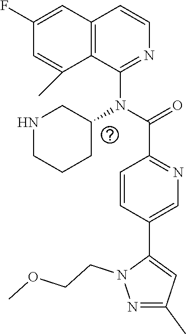 [SMILES: COCCn1nc(C)cc1-c1ccc(C(=O)N(c2nccc3cc(F)cc(C)c23)[C@@H]2CCCNC2)nc1] |
| 730 | (R)-N-(6-chloro-8-methylisoquinolin-1- yl)-5-(5-ethyl-1,3,4-thiadiazol-2-yl)-N- (piperidin-3-yl)picolinamide | 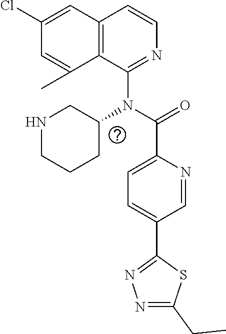 [SMILES: CCc1nnc(-c2ccc(C(=O)N(c3nccc4cc(Cl)cc(C)c34)[C@@H]3CCCNC3)nc2)s1] |
| 731 | (R)-4-((4-(1-chloro-3-methoxyprop-1-en- 1-yl)pyrimidin-2-yl)amino)-2-fluoro-N-(8- methylisoquinolin-1-yl)-N-(piperidin-3- yl)benzamide | 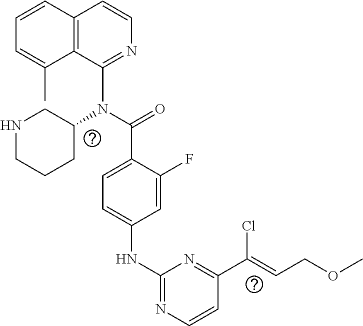 [SMILES: COC/C=C(\Cl)c1ccnc(Nc2ccc(C(=O)N(c3nccc4cccc(C)c34)[C@@H]3CCCNC3)c(F)c2)n1] |
| 732 | (R)-2-fluoro-4-(5-methyl-1,3,4-thiadiazol- 2-yl)-N-(8-methyl-6-(2-methylthiazol-5- yl)isoquinolin-1-yl)-N-(piperidin-3- yl)benzamide | 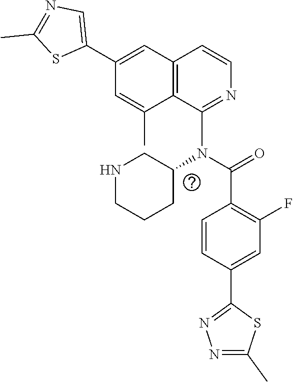 [SMILES: Cc1ncc(-c2cc(C)c3c(N(C(=O)c4ccc(-c5nnc(C)s5)cc4F)[C@@H]4CCCNC4)nccc3c2)s1] |
| 733 | (R)-2-fluoro-4-(5-methyl-1,3,4-thiadiazol- 2-yl)-N-(8-methyl-6-(thiazol-2- yl)isoquinolin-1-yl)-N-(piperidin-3- yl)benzamide | 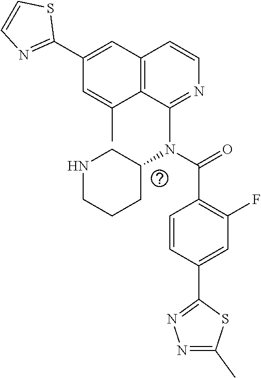 [SMILES: Cc1nnc(-c2ccc(C(=O)N(c3nccc4cc(-c5nccs5)cc(C)c34)[C@@H]3CCCNC3)c(F)c2)s1] |
| 734 | (R)-2-fluoro-4-((4-((2- hydroxyethyl)amino)pyrimidin-2- yl)amino)-N-(8-methylisoquinolin-1-yl)-N- (piperidin-3-yl)benzamide | 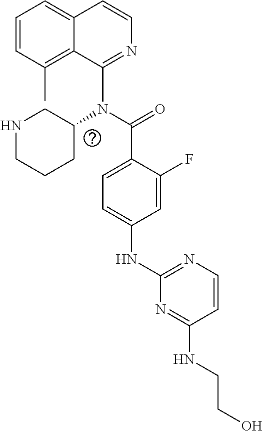 [SMILES: Cc1cccc2ccnc(N(C(=O)c3ccc(Nc4nccc(NCCO)n4)cc3F)[C@@H]3CCCNC3)c12] |
| 735 | (R)-2-fluoro-4-((4-hydroxypyrimidin-2- yl)amino)-N-(8-methylisoquinolin-1-yl)-N- (piperidin-3-yl)benzamide | 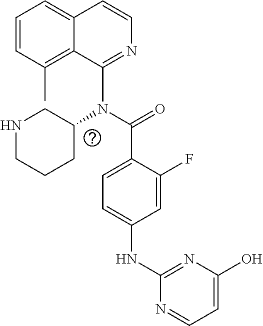 [SMILES: Cc1cccc2ccnc(N(C(=O)c3ccc(Nc4nccc(O)n4)cc3F)[C@@H]3CCCNC3)c12] |
| 736 | (R)-2-fluoro-4-((4-(2- hydroxyethyl)pyrimidin-2-yl)amino)-N-(8- methylisoquinolin-1-yl)-N-(piperidin-3- yl)benzamide | 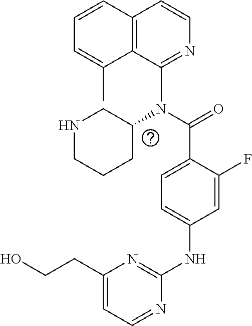 [SMILES: Cc1cccc2ccnc(N(C(=O)c3ccc(Nc4nccc(CCO)n4)cc3F)[C@@H]3CCCNC3)c12] |
| 737 | (R)-4-(5,5-dimethyl-4-oxo-4,5- dihydroisoxazol-3-yl)-2-fluoro-N-(2-(3- hydroxyprop-1-yn-1-yl)thieno[3,2- c]pyridin-4-yl)-N-(piperidin-3- yl)benzamide | 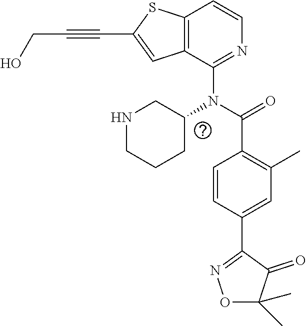 [SMILES: Cc1cc(C2=NOC(C)(C)C2=O)ccc1C(=O)N(c1nccc2sc(C#CCO)cc12)[C@@H]1CCCNC1] |
| 738 | (R)-2-fluoro-N-(piperidin-3-yl)-4- (pyrimidin-2-ylamino)-N-(6-(pyrimidin-2- ylamino)isoquinolin-1-yl)benzamide | 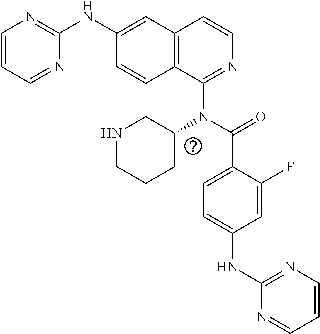 [SMILES: O=C(c1ccc(Nc2ncccn2)cc1F)N(c1nccc2cc(Nc3ncccn3)ccc12)[C@@H]1CCCNC1] |
| 739 | (R)-4-(3H-[1,2,3]triazolo[4,5-b]pyridin-3- yl)-2-fluoro-N-(2-methylthieno[3,2- c]pyridin-4-yl)-N-(piperidin-3- yl)benzamide | 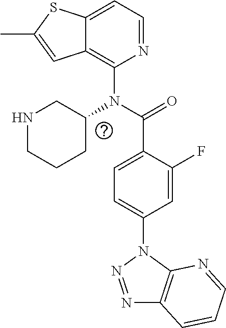 [SMILES: Cc1cc2c(N(C(=O)c3ccc(-n4nnc5cccnc54)cc3F)[C@@H]3CCCNC3)nccc2s1] |
| 740 | (R)-4-(3H-[1,2,3]triazolo[4,5-b]pyridin-3- yl)-N-(2-cyclopropylthieno[3,2-c]pyridin- 4-yl)-2-fluoro-N-(piperidin-3- yl)benzamide | 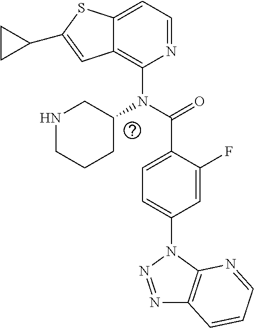 [SMILES: O=C(c1ccc(-n2nnc3cccnc32)cc1F)N(c1nccc2sc(C3CC3)cc12)[C@@H]1CCCNC1] |
| 741 | (R)-2-fluoro-N-(8-methylisoquinolin-1-yl)- 4-((4-(morpholinomethyl)pyrimidin-2- yl)amino)-N-(piperidin-3-yl)benzamide | 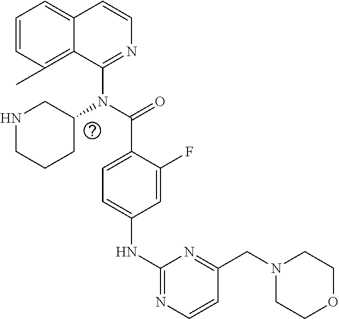 [SMILES: Cc1cccc2ccnc(N(C(=O)c3ccc(Nc4nccc(CN5CCOCC5)n4)cc3F)[C@@H]3CCCNC3)c12] |
| 742 | (R)-2-fluoro-4-((4-((2- methoxyethyl)(methyl)amino)pyrimidin-2- yl)amino)-N-(8-methylisoquinolin-1-yl)-N- (piperidin-3-yl)benzamide | 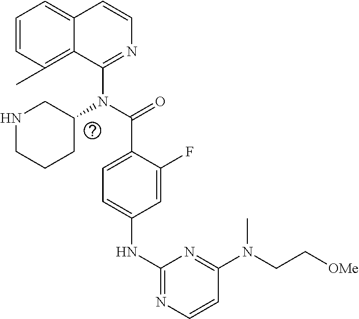 [SMILES: COCCN(C)c1ccnc(Nc2ccc(C(=O)N(c3nccc4cccc(C)c34)[C@@H]3CCCNC3)c(F)c2)n1] |
| 743 | (R)-2-fluoro-4-((4-((2- hydroxyethyl)(methyl)amino)pyrimidin-2- yl)amino)-N-(8-methylisoquinolin-1-yl)-N- (piperidin-3-yl)benzamide | 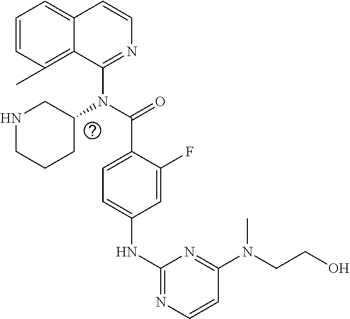 [SMILES: Cc1cccc2ccnc(N(C(=O)c3ccc(Nc4nccc(N(C)CCO)n4)cc3F)[C@@H]3CCCNC3)c12] |
| 744 | (R)-2-fluoro-4-((4-((2- methoxyethyl)amino)pyrimidin-2- yl)amino)-N-(8-methylisoquinolin-1-yl)-N- (piperidin-3-yl)benzamide | 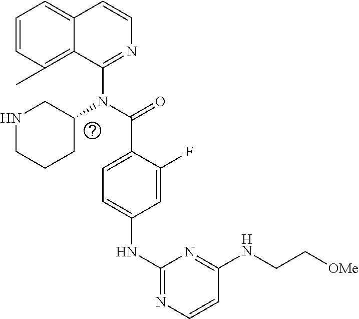 [SMILES: COCCNc1ccnc(Nc2ccc(C(=O)N(c3nccc4cccc(C)c34)[C@@H]3CCCNC3)c(F)c2)n1] |
| 745 | (R)-2-fluoro-4-((4- (methyl(propyl)amino)pyrimidin-2- yl)amino)-N-(8-methylisoquinolin-1-yl)-N- (piperidin-3-yl)benzamide | 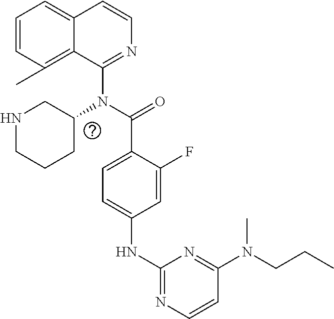 [SMILES: CCCN(C)c1ccnc(Nc2ccc(C(=O)N(c3nccc4cccc(C)c34)[C@@H]3CCCNC3)c(F)c2)n1] |
| 746 | (R)-2-fluoro-N-(8-methylisoquinolin-1-yl)- N-(piperidin-3-yl)-4-((4- (propylamino)pyrimidin-2- yl)amino)benzamide | 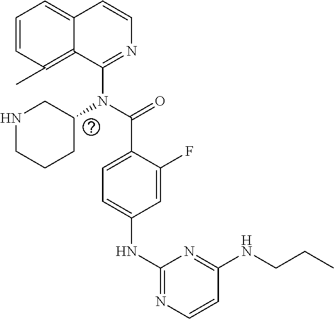 [SMILES: CCCNc1ccnc(Nc2ccc(C(=O)N(c3nccc4cccc(C)c34)[C@@H]3CCCNC3)c(F)c2)n1] |
| 747 | (R)-3-methyl-N-(8-methylisoquinolin-1- yl)-N-(piperidin-3-yl)-4-(pyrimidin-2- ylamino)benzamide | 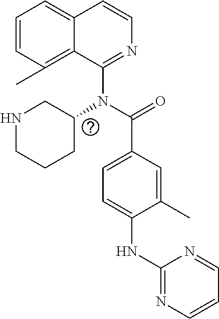 [SMILES: Cc1cc(C(=O)N(c2nccc3cccc(C)c23)[C@@H]2CCCNC2)ccc1Nc1ncccn1] |
| 748 | (R,E)-2-fluoro-4-((4-(3-hydroxyprop-1-en- 1-yl)pyrimidin-2-yl)amino)-N-(8- methylisoquinolin-1-yl)-N-(piperidin-3- yl)benzamide | 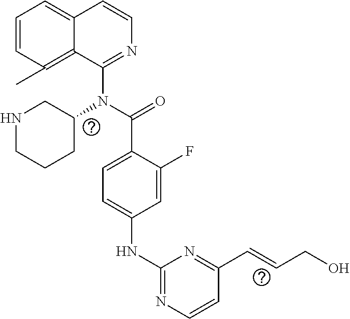 [SMILES: Cc1cccc2ccnc(N(C(=O)c3ccc(Nc4nccc(/C=C/CO)n4)cc3F)[C@@H]3CCCNC3)c12] |
| 749 | (R,E)-2-fluoro-4-((4-(3-methoxyprop-1-en- 1-yl)pyrimidin-2-yl)amino)-N-(8- methylisoquinolin-1-yl)-N-(piperidin-3- yl)benzamide | 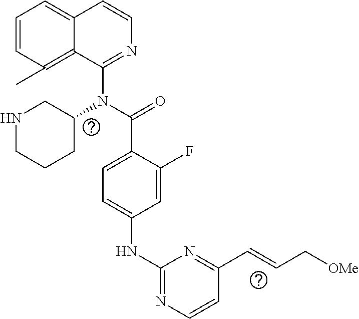 [SMILES: COC/C=C/c1ccnc(Nc2ccc(C(=O)N(c3nccc4cccc(C)c34)[C@@H]3CCCNC3)c(F)c2)n1] |
| 750 | (R)-2-fluoro-N-(8-methylisoquinolin-1-yl)- N-(piperidin-3-yl)-4-((4-(tetrahydro-2H- pyran-4-yl)pyrimidin-2- yl)amino)benzamide | 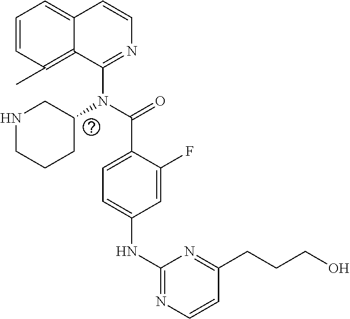 [SMILES: Cc1cccc2ccnc(N(C(=O)c3ccc(Nc4nccc(CCCO)n4)cc3F)[C@@H]3CCCNC3)c12] |
| 751 | (R)-2-fluoro-4-((4-(3-methoxyprop-1-yn-1- yl)pyrimidin-2-yl)amino)-N-(8- methylisoquinolin-1-yl)-N-(piperidin-3- yl)benzamide | 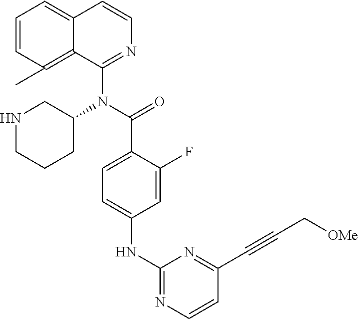 [SMILES: COCC#Cc1ccnc(Nc2ccc(C(=O)N(c3nccc4cccc(C)c34)[C@@H]3CCCNC3)c(F)c2)n1] |
| 752 | (R)-2-fluoro-4-((4-(3- methoxypropyl)pyrimidin-2-yl)amino)-N- (8-methylisoquinolin-1-yl)-N-(piperidin-3- yl)benzamide hydrochloride | 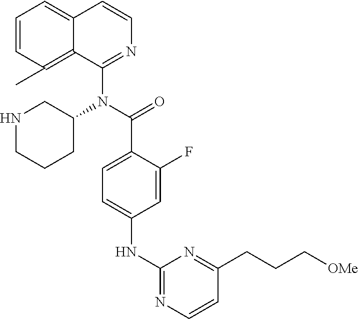 [SMILES: COCCCc1ccnc(Nc2ccc(C(=O)N(c3nccc4cccc(C)c34)[C@@H]3CCCNC3)c(F)c2)n1] |
| 753 | (R)-4-((4-ethyl-5-fluoropyrimidin-2- yl)amino)-2-fluoro-N-(8- methylisoquinolin-1-yl)-N-(piperidin-3- yl)benzamide | 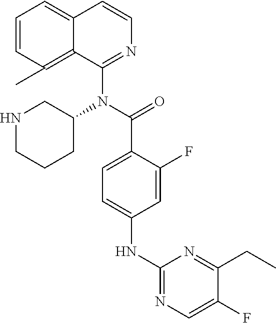 [SMILES: CCc1nc(Nc2ccc(C(=O)N(c3nccc4cccc(C)c34)[C@@H]3CCCNC3)c(F)c2)ncc1F] |
| 754 | (R)-2-fluoro-4-((5-fluoro-4- methylpyrimidin-2-yl)amino)-N-(8- methylisoquinolin-1-yl)-N-(piperidin-3- yl)benzamide | 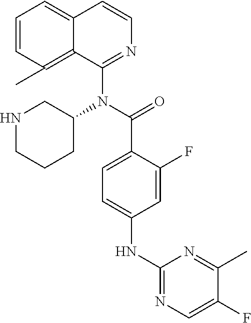 [SMILES: Cc1nc(Nc2ccc(C(=O)N(c3nccc4cccc(C)c34)[C@@H]3CCCNC3)c(F)c2)ncc1F] |
| 755 | (R)-N-(6-chloro-8-methylisoquinolin-1- yl)-4-(5-methyl-1,3,4-thiadiazol-2-yl)-N- (piperidin-3-yl)benzamide | 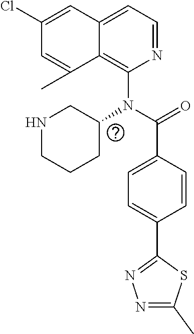 [SMILES: Cc1nnc(-c2ccc(C(=O)N(c3nccc4cc(Cl)cc(C)c34)[C@@H]3CCCNC3)cc2)s1] |
| 756 | (R)-4-(1H-benzo[d]imidazol-1-yl)-2- fluoro-N-(2-methylthieno[3,2-c]pyridin-4- yl)-N-(piperidin-3-yl)benzamide | 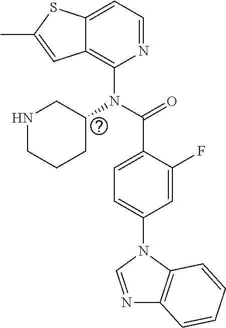 [SMILES: Cc1cc2c(N(C(=O)c3ccc(-n4cnc5ccccc54)cc3F)[C@@H]3CCCNC3)nccc2s1] |
| 757 | (R)-2-methyl-4-(5-methyl-1,3,4-thiadiazol- 2-yl)-N-(3-methylthieno[3,2-c]pyridin-4- yl)-N-(piperidin-3-yl)benzamide | 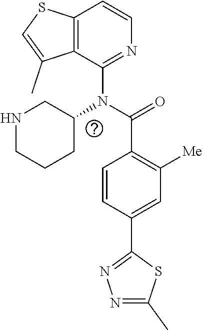 [SMILES: Cc1nnc(-c2ccc(C(=O)N(c3nccc4scc(C)c34)[C@@H]3CCCNC3)c(C)c2)s1] |
| 758 | (R)-2-chloro-4-(1-methyl-1H-1,2,3-triazol- 4-yl)-N-(3-methylthieno[3,2-c]pyridin-4- yl)-N-(piperidin-3-yl)benzamide | 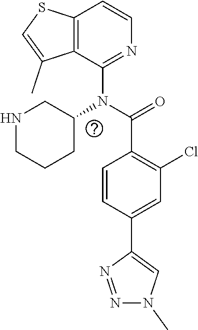 [SMILES: Cc1csc2ccnc(N(C(=O)c3ccc(-c4cn(C)nn4)cc3Cl)[C@@H]3CCCNC3)c12] |
| 759 | (R)-2-fluoro-N-(5-fluoro-8- methylisoquinolin-1-yl)-4-(5-methyl-1,3,4- thiadiazol-2-yl)-N-(piperidin-3- yl)benzamide | 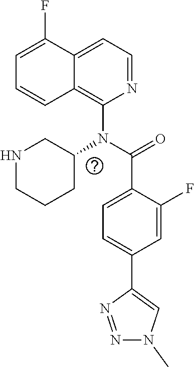 [SMILES: Cn1cc(-c2ccc(C(=O)N(c3nccc4c(F)cccc34)[C@@H]3CCCNC3)c(F)c2)nn1] |
| 760 | (R)-2-fluoro-N-(5-fluoro-8- methylisoquinolin-1-yl)-4-(5-methyl-1,3,4- thiadiazol-2-yl)-N-(piperidin-3- yl)benzamide | 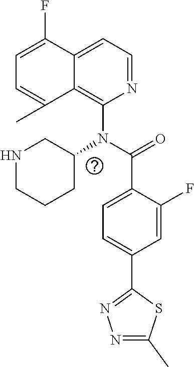 [SMILES: Cc1nnc(-c2ccc(C(=O)N(c3nccc4c(F)ccc(C)c34)[C@@H]3CCCNC3)c(F)c2)s1] |
| 761 | (R)-2-fluoro-N-(5-fluoro-8- methylisoquinolin-1-yl)-4-(1-methyl-1H- 1,2,3-triazol-4-yl)-N-(piperidin-3- yl)benzamide | 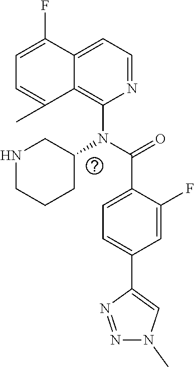 [SMILES: Cc1ccc(F)c2ccnc(N(C(=O)c3ccc(-c4cn(C)nn4)cc3F)[C@@H]3CCCNC3)c12] |
| 762 | (R)-2-fluoro-N-(6-methoxyisoquinolin-1- yl)-4-(5-methyl-1,3,4-thiadiazol-2-yl)-N- (piperidin-3-yl)benzamide | 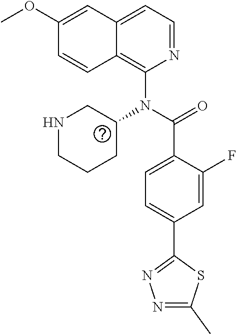 [SMILES: COc1ccc2c(N(C(=O)c3ccc(-c4nnc(C)s4)cc3F)[C@@H]3CCCNC3)nccc2c1] |
| 763 | (R)-N-(6-chloro-8-methylisoquinolin-1- yl)-4-(1,5-dimethyl-1H-1,2,3-triazol-4-yl)- 2-fluoro-N-(piperidin-3-yl)benzamide | 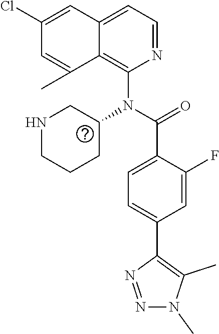 [SMILES: Cc1cc(Cl)cc2ccnc(N(C(=O)c3ccc(-c4nnn(C)c4C)cc3F)[C@@H]3CCCNC3)c12] |
| 764 | (R)-2-chloro-N-(6-chloro-8- methylisoquinolin-1-yl)-4-(5-methyl-1,3,4- oxadiazol-2-yl)-N-(piperidin-3- yl)benzamide | 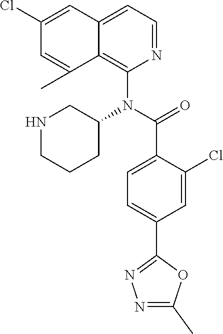 [SMILES: Cc1nnc(-c2ccc(C(=O)N(c3nccc4cc(Cl)cc(C)c34)[C@@H]3CCCNC3)c(Cl)c2)o1] |
| 765 | (R)-N-(6-chloro-8-methylisoquinolin-1- yl)-2-fluoro-4-(5-methyl-1,3,4-oxadiazol- 2-yl)-N-(piperidin-3-yl)benzamide | 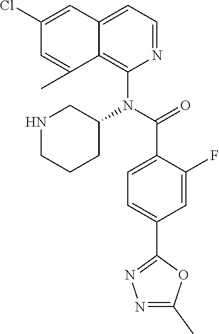 [SMILES: Cc1nnc(-c2ccc(C(=O)N(c3nccc4cc(Cl)cc(C)c34)[C@@H]3CCCNC3)c(F)c2)o1] |
| 766 | (R)-N-(6-chloro-8-methylisoquinolin-1- yl)-2-fluoro-4-(5-(methyl-d3)-1,3,4- thiadiazol-2-yl)-N-(piperidin-3- yl)benzamide | 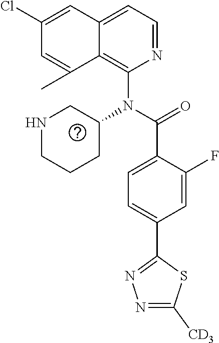 [SMILES: [2H]C([2H])([2H])c1nnc(-c2ccc(C(=O)N(c3nccc4cc(Cl)cc(C)c34)[C@@H]3CCCNC3)c(F)c2)s1] |
| 767 | (R)-2-fluoro-N-(6-fluoro-8- methylisoquinolin-1-yl)-4-(5-(methyl-d3)- 1,3,4-thiadiazol-2-yl)-N-(piperidin-3- yl)benzamide | 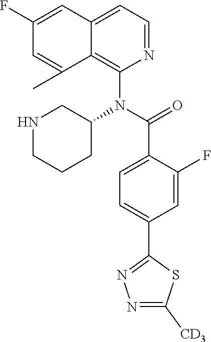 [SMILES: [2H]C([2H])([2H])c1nnc(-c2ccc(C(=O)N(c3nccc4cc(F)cc(C)c34)[C@@H]3CCCNC3)c(F)c2)s1] |
| 768 | (R)-4-(5-chloropyridazin-3-yl)-2-fluoro-N- (3-methylthieno[3,2-c]pyridin-4-yl)-N- (piperidin-3-yl)benzamide | 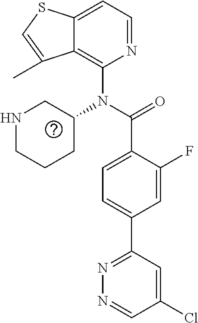 [SMILES: Cc1csc2ccnc(N(C(=O)c3ccc(-c4cc(Cl)cnn4)cc3F)[C@@H]3CCCNC3)c12] |
| 769 | (R)-N-(6-chloro-8-methylisoquinolin-1- yl)-5-(5-chloropyridazin-3-yl)-N- (piperidin-3-yl)picolinamide | 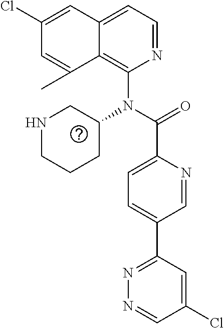 [SMILES: Cc1cc(Cl)cc2ccnc(N(C(=O)c3ccc(-c4cc(Cl)cnn4)cn3)[C@@H]3CCCNC3)c12] |
| 770 | (R)-4-(1,5-dimethyl-1H-1,2,3-triazol-4-yl)- N-(6-fluoro-8-methylisoquinolin-1-yl)-N- (piperidin-3-yl)benzamide | 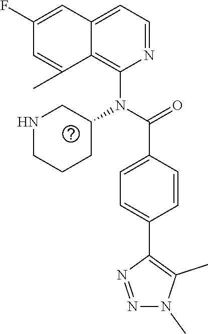 [SMILES: Cc1cc(F)cc2ccnc(N(C(=O)c3ccc(-c4nnn(C)c4C)cc3)[C@@H]3CCCNC3)c12] |
| 771 | (R)-N-(6-chloro-8-methylisoquinolin-1- yl)-4-(1,5-dimethyl-1H-1,2,3-triazol-4-yl)- N-(piperidin-3-yl)benzamide | 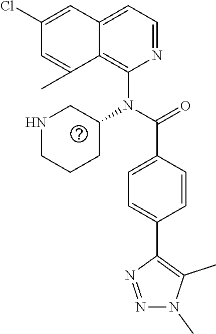 [SMILES: Cc1cc(Cl)cc2ccnc(N(C(=O)c3ccc(-c4nnn(C)c4C)cc3)[C@@H]3CCCNC3)c12] |
| 772 | (R)-N-(6-chloro-8-methylisoquinolin-1- yl)-4-(5-methyl-1,3,4-oxadiazol-2-yl)-N- (piperidin-3-yl)benzamide | 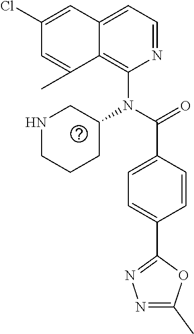 [SMILES: Cc1nnc(-c2ccc(C(=O)N(c3nccc4cc(Cl)cc(C)c34)[C@@H]3CCCNC3)cc2)o1] |
| 773 | (R)-N-(6-chloro-8-methylisoquinolin-1- yl)-4-(3-methyl-1,2,4-oxadiazol-5-yl)-N- (piperidin-3-yl)benzamide | 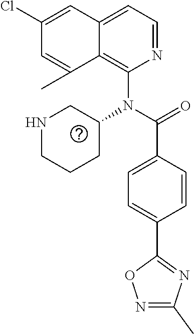 [SMILES: Cc1noc(-c2ccc(C(=O)N(c3nccc4cc(Cl)cc(C)c34)[C@@H]3CCCNC3)cc2)n1] |
| 774 | (R)-N-(6-fluoro-8-methylisoquinolin-1-yl)- 4-(3-methyl-1,2,4-thiadiazol-5-yl)-N- (piperidin-3-yl)benzamide | 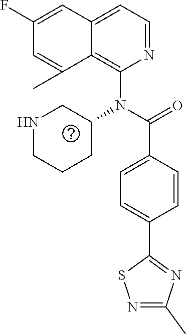 [SMILES: Cc1nsc(-c2ccc(C(=O)N(c3nccc4cc(F)cc(C)c34)[C@@H]3CCCNC3)cc2)n1] |
| 775 | (R)-N-(6-fluoro-8-methylisoquinolin-1-yl)- 4-(3-methyl-1,2,4-oxadiazol-5-yl)-N- (piperidin-3-yl)benzamide | 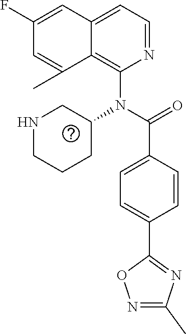 [SMILES: Cc1noc(-c2ccc(C(=O)N(c3nccc4cc(F)cc(C)c34)[C@@H]3CCCNC3)cc2)n1] |
| 776 | (R)-2-fluoro-N-(6-fluoro-8- methylisoquinolin-1-yl)-4-(5-methyl-1,3,4- oxadiazol-2-yl)-N-(piperidin-3- yl)benzamide | 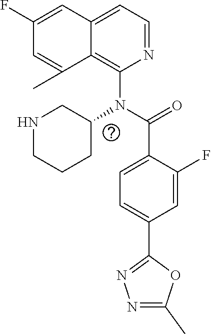 [SMILES: Cc1nnc(-c2ccc(C(=O)N(c3nccc4cc(F)cc(C)c34)[C@@H]3CCCNC3)c(F)c2)o1] |
| 777 | (R)-2-methyl-4-(3-methyl-1,2,4-oxadiazol- 5-yl)-N-(3-methylthieno[3,2-c]pyridin-4- yl)-N-(piperidin-3-yl)benzamide | 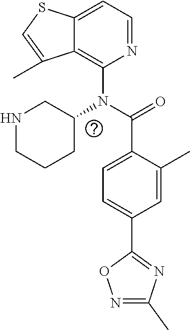 |
| 778 | (R)-2-fluoro-4-(3-methyl-1,2,4-oxadiazol- 5-yl)-N-(3-methylthieno[3,2-c]pyridin-4- yl)-N-(piperidin-3-yl)benzamide | 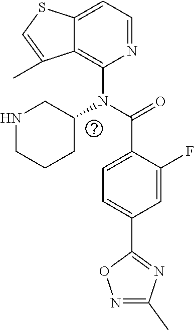 [SMILES: Cc1noc(-c2ccc(C(=O)N(c3nccc4scc(C)c34)[C@@H]3CCCNC3)c(F)c2)n1] |
| 779 | (R)-2-fluoro-4-(3-methyl-1,2,4-thiadiazol- 5-yl)-N-(3-methylthieno[3,2-c]pyridin-4- yl)-N-(piperidin-3-yl)benzamide | 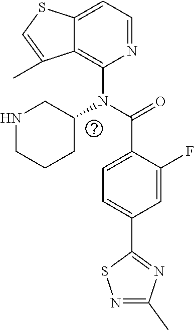 [SMILES: Cc1nsc(-c2ccc(C(=O)N(c3nccc4scc(C)c34)[C@@H]3CCCNC3)c(F)c2)n1] |
| 780 | (R)-2-fluoro-4-(5-(methyl-d3)-1,3,4- thiadiazol-2-yl)-N-(3-methylthieno[3,2- c]pyridin-4-yl)-N-(piperidin-3- yl)benzamide | 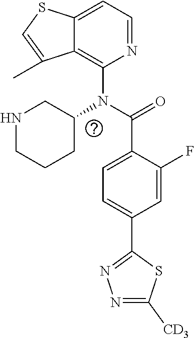 [SMILES: [2H]C([2H])([2H])c1nnc(-c2ccc(C(=O)N(c3nccc4scc(C)c34)[C@@H]3CCCNC3)c(F)c2)s1] |
| 781 | (R)-2-fluoro-4-(5-methyl-1,3,4-thiadiazol- 2-yl)-N-(3-methylthieno[3,2-c]pyridin-4- yl)-N-(piperidin-3-yl)benzamide | 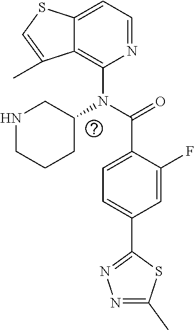 [SMILES: Cc1nnc(-c2ccc(C(=O)N(c3nccc4scc(C)c34)[C@@H]3CCCNC3)c(F)c2)s1] |
| 782 | (R)-4-(1,5-dimethyl-1H-1,2,3-triazol-4-yl)- 2-fluoro-N-(3-methylthieno[3,2-c]pyridin- 4-yl)-N-(piperidin-3-yl)benzamide | 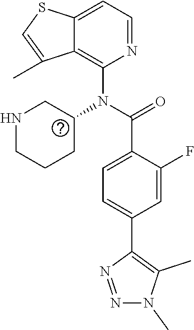 |
| 783 | (R)-5-(5-methyl-1,3,4-thiadiazol-2-yl)-N- (8-methylisoquinolin-1-yl)-N-(piperidin-3- yl)picolinamide | 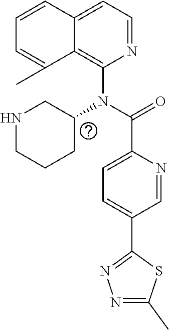 [SMILES: Cc1nnc(-c2ccc(C(=O)N(c3nccc4cccc(C)c34)[C@@H]3CCCNC3)nc2)s1] |
| 784 | (R)-N-(6-fluoro-8-methylisoquinolin-1-yl)- 5-(5-methyl-1,3,4-thiadiazol-2-yl)-N- (piperidin-3-yl)picolinamide | 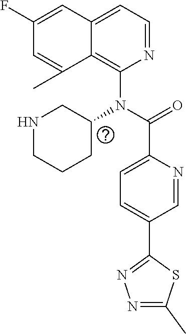 [SMILES: Cc1nnc(-c2ccc(C(=O)N(c3nccc4cc(F)cc(C)c34)[C@@H]3CCCNC3)nc2)s1] |
| 785 | (R)-N-(6-fluoro-8-methylisoquinolin-1-yl)- 5-(1-methyl-1H-1,2,3-triazol-4-yl)-N- (piperidin-3-yl)picolinamide | 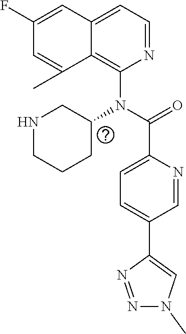 [SMILES: Cc1cc(F)cc2ccnc(N(C(=O)c3ccc(-c4cn(C)nn4)cn3)[C@@H]3CCCNC3)c12] |
| 786 | (R)-4-((4-(benzylamino) pyrimidin-2-yl) amino)-2-fluoro-N-(8-methylisoquinolin-1- yl)-N-(piperidin-3-yl) benzamide hydrochloride | 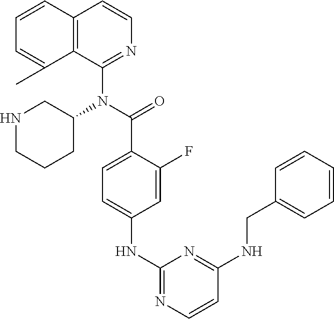 [SMILES: Cc1cccc2ccnc(N(C(=O)c3ccc(Nc4nccc(NCc5ccccc5)n4)cc3F)[C@@H]3CCCNC3)c12] |
| 787 | (R)-2-fluoro-N-(6-fluoroisoquinolin-1-yl)- 4-((5-fluoropyrimidin-2-yl)amino)-N- (piperidin-3-yl)benzamide | 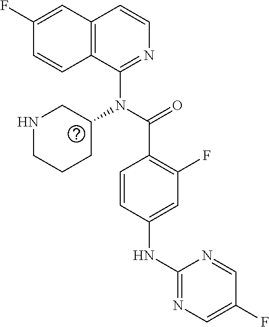 [SMILES: O=C(c1ccc(Nc2ncc(F)cn2)cc1F)N(c1nccc2cc(F)ccc12)[C@@H]1CCCNC1] |
| 788 | (R)-N-(6-chloroisoquinolin-1-yl)-2-fluoro- 4-(5-methyl-1,3,4-oxadiazol-2-yl)-N- (piperidin-3-yl)benzamide | 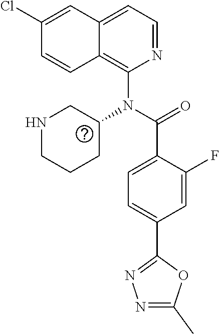 [SMILES: Cc1nnc(-c2ccc(C(=O)N(c3nccc4cc(Cl)ccc34)[C@@H]3CCCNC3)c(F)c2)o1] |
| 789 | (R)-4-(5-cyclopropyl-1,3,4-oxadiazol-2- yl)-N-(8-methylisoquinolin-1-yl)-N- (piperidin-3-yl)benzamide | 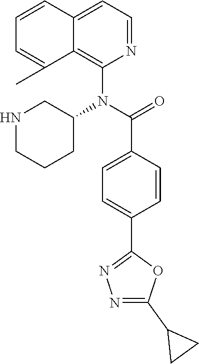 [SMILES: Cc1cccc2ccnc(N(C(=O)c3ccc(-c4nnc(C5CC5)o4)cc3)[C@@H]3CCCNC3)c12] |
| 790 | (R)-4-(5-cyclopropyl-1,3,4-thiadiazol-2- yl)-N-(8-methylisoquinolin-1-yl)-N- (piperidin-3-yl)benzamide | 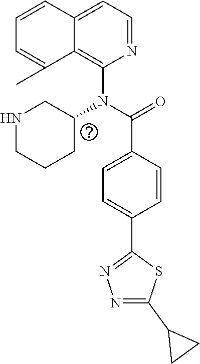 [SMILES: Cc1cccc2ccnc(N(C(=O)c3ccc(-c4nnc(C5CC5)s4)cc3)[C@@H]3CCCNC3)c12] |
| 791 | (R)-4-(5-ethyl-1,3,4-thiadiazol-2-yl)-N-(8- methylisoquinolin-1-yl)-N-(piperidin-3- yl)benzamide | 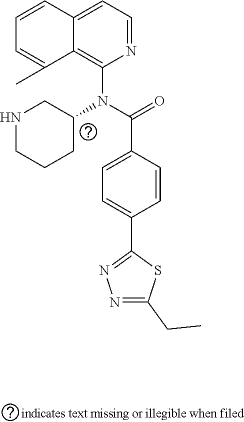 [SMILES: CCc1nnc(-c2ccc(C(=O)N(c3nccc4cccc(C)c34)[C@@H]3CCCNC3)cc2)s1] |
| 792 | (R)-4-(5-methyl-1,3,4-thiadiazol-2-yl)-N- (8-methylisoquinolin-1-yl)-N-(piperidin-3- yl)benzamide | 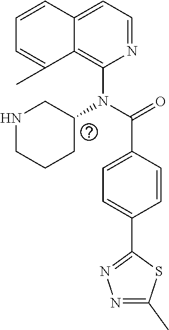 [SMILES: Cc1nnc(-c2ccc(C(=O)N(c3nccc4cccc(C)c34)[C@@H]3CCCNC3)cc2)s1] |
| 793 | (R)-N-(6-chloro-8-methylisoquinolin-1- yl)-2-fluoro-N-(piperidin-3-yl)-4- (pyrimidin-2-ylamino)benzamide | 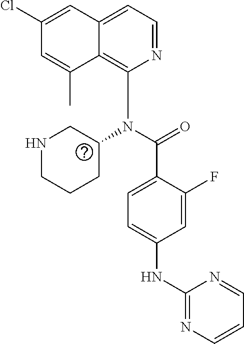 [SMILES: Cc1cc(Cl)cc2ccnc(N(C(=O)c3ccc(Nc4ncccn4)cc3F)[C@@H]3CCCNC3)c12] |
| 794 | (R)-N-(6-chloroisoquinolin-1-yl)-2-fluoro- N-(piperidin-3-yl)-4-(pyrimidin-2- ylamino)benzamide | 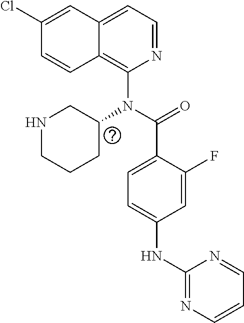 [SMILES: O=C(c1ccc(Nc2ncccn2)cc1F)N(c1nccc2cc(Cl)ccc12)[C@@H]1CCCNC1] |
| 795 | (R)-N-(3-chlorothieno[3,2-c]pyridin-4-yl)- 2-fluoro-4-(1-methyl-1H-1,2,3-triazol-4- yl)-N-(piperidin-3-yl)benzamide | 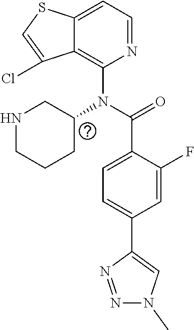 [SMILES: Cn1cc(-c2ccc(C(=O)N(c3nccc4scc(Cl)c34)[C@@H]3CCCNC3)c(F)c2)nn1] |
| 796 | (R)-4-(3-methylisoxazol-5-yl)-N-(3- methylthieno[3,2-c]pyridin-4-yl)-N- (piperidin-3-yl)piperidine-1-carboxamide | 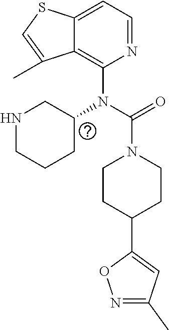 |
| 797 | (R)-5-(3-methylisoxazol-5-yl)-N-(3- methylthieno[3,2-c]pyridin-4-yl)-N- (piperidin-3-yl)picolinamide | 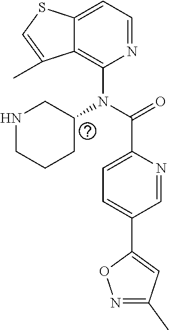 |
| 798 | (R)-4-(5-methyl-1,3,4-thiadiazol-2-yl)-N- (3-methylthieno[3,2-c]pyridin-4-yl)-N- (piperidin-3-yl)benzamide | 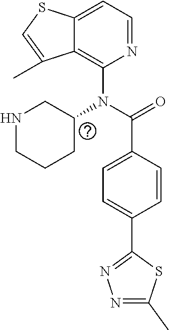 |
| 799 | (R)-4-(1-methyl-1H-1,2,3-triazol-4-yl)-N- (3-methylthieno[3,2-c]pyridin-4-yl)-N- (piperidin-3-yl)benzamide | 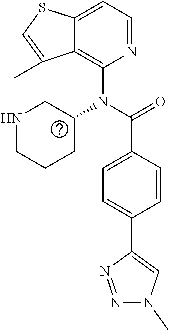 |
| 800 | (R)-N-(8-chloroisoquinolin-1-yl)-4-(1- methyl-1H-1,2,3-triazol-4-yl)-N- (piperidin-3-yl)benzamide | 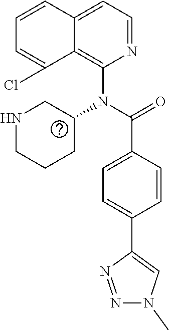 [SMILES: Cn1cc(-c2ccc(C(=O)N(c3nccc4cccc(Cl)c34)[C@@H]3CCCNC3)cc2)nn1] |
| 801 | (R)-N-(6-chloro-8-methylisoquinolin-1- yl)-5-(5-methyl-1,3,4-thiadiazol-2-yl)-N- (piperidin-3-yl)picolinamide | 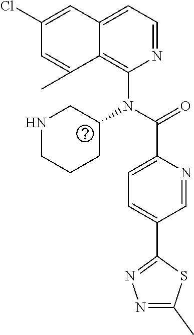 [SMILES: Cc1nnc(-c2ccc(C(=O)N(c3nccc4cc(Cl)cc(C)c34)[C@@H]3CCCNC3)nc2)s1] |
| 802 | (R)-N-(6-chloro-8-methylisoquinolin-1- yl)-4-(1-methyl-1H-1,2,3-triazol-4-yl)-N- (piperidin-3-yl)benzamide | 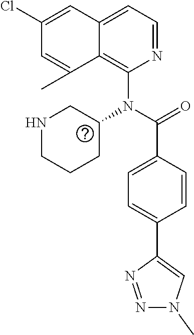 [SMILES: Cc1cc(Cl)cc2ccnc(N(C(=O)c3ccc(-c4cn(C)nn4)cc3)[C@@H]3CCCNC3)c12] |
| 803 | (R)-N-(6-fluoroisoquinolin-1-yl)-5-(1- methyl-1H-1,2,3-triazol-4-yl)-N- (piperidin-3-yl)picolinamide | 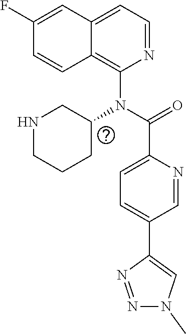 [SMILES: Cn1cc(-c2ccc(C(=O)N(c3nccc4cc(F)ccc34)[C@@H]3CCCNC3)nc2)nn1] |
| 804 | (R)-N-(6-fluoroisoquinolin-1-yl)-4-(5- methyl-1,3,4-thiadiazol-2-yl)-N-(piperidin- 3-yl)benzamide | 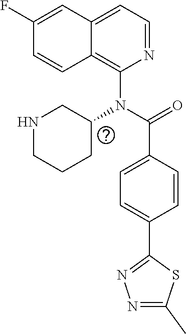 [SMILES: Cc1nnc(-c2ccc(C(=O)N(c3nccc4cc(F)ccc34)[C@@H]3CCCNC3)cc2)s1] |
| 805 | (R)-N-(6-fluoroisoquinolin-1-yl)-4-(1- methyl-1H-1,2,3-triazol-4-yl)-N- (piperidin-3-yl)benzamide | 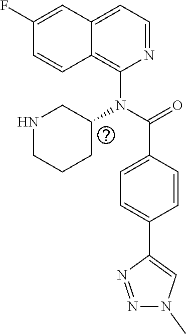 [SMILES: Cn1cc(-c2ccc(C(=O)N(c3nccc4cc(F)ccc34)[C@@H]3CCCNC3)cc2)nn1] |
| 806 | (R)-N-(6-chloroisoquinolin-1-yl)-N- (piperidin-3-yl)-5-(pyridazin-3- yl)picolinamide | 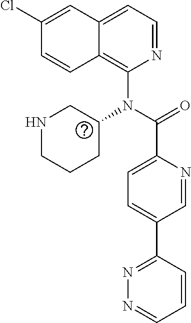 [SMILES: O=C(c1ccc(-c2cccnn2)cn1)N(c1nccc2cc(Cl)ccc12)[C@@H]1CCCNC1] |
| 807 | (R)-N-(6-chloroisoquinolin-1-yl)-4-(5- methyl-1,3,4-thiadiazol-2-yl)-N-(piperidin- 3-yl)benzamide | 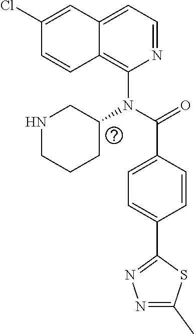 [SMILES: Cc1nnc(-c2ccc(C(=O)N(c3nccc4cc(Cl)ccc34)[C@@H]3CCCNC3)cc2)s1] |
| 808 | (R)-N-(6-chloroisoquinolin-1-yl)-4-(1- methyl-1H-1,2,3-triazol-4-yl)-N- (piperidin-3-yl)benzamide | 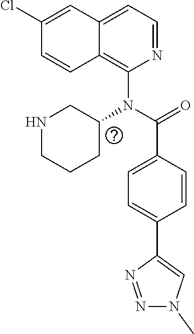 [SMILES: Cn1cc(-c2ccc(C(=O)N(c3nccc4cc(Cl)ccc34)[C@@H]3CCCNC3)cc2)nn1] |
| 809 | (R)-5-(1-methyl-1H-1,2,3-triazol-4-yl)-N- (8-methylisoquinolin-1-yl)-N-(piperidin-3- yl)picolinamide | 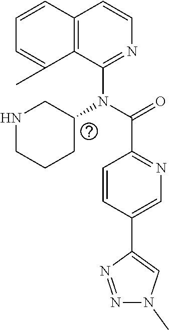 [SMILES: Cc1cccc2ccnc(N(C(=O)c3ccc(-c4cn(C)nn4)cn3)[C@@H]3CCCNC3)c12] |
| 810 | (R)-5-(5-methyl-1,3,4-thiadiazol-2-yl)-N- (3-methylthieno[3,2-c]pyridin-4-yl)-N- (piperidin-3-yl)picolinamide | 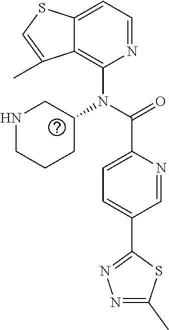 |
| 811 | (R)-5-(1-methyl-1H-1,2,3-triazol-4-yl)-N- (3-methylthieno[3,2-c]pyridin-4-yl)-N- (piperidin-3-yl)picolinamide | 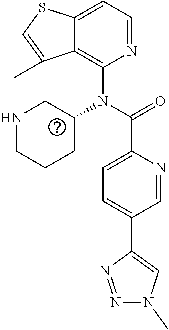 |
| 812 | (R)-N-(6-chloro-8-methylisoquinolin-1- yl)-5-(1-methyl-1H-1,2,3-triazol-4-yl)-N- (piperidin-3-yl)picolinamide | 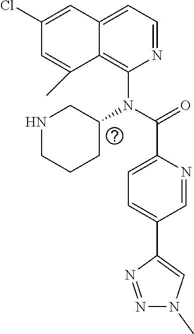 [SMILES: Cc1cc(Cl)cc2ccnc(N(C(=O)c3ccc(-c4cn(C)nn4)cn3)[C@@H]3CCCNC3)c12] |
| 813 | (R)-4-(2-fluoro-4-(1-methyl-1H-1,2,3- triazol-4-yl)-N-(piperidin-3- yl)benzamido)-N-(pyridin-4-yl)thieno[3,2- c]pyridine-2-carboxamide | 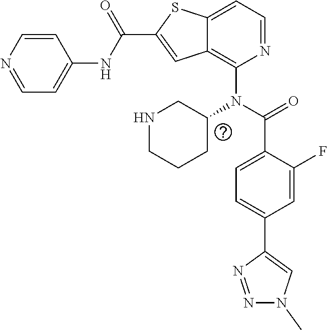 [SMILES: Cn1cc(-c2ccc(C(=O)N(c3nccc4sc(C(=O)Nc5ccncc5)cc34)[C@@H]3CCCNC3)c(F)c2)nn1] |
| 814 | (R)-N-(6-chloro-8-methylisoquinolin-1- yl)-4-(1,5-dimethyl-1H-pyrazol-4-yl)-2- fluoro-N-(piperidin-3-yl)benzamide | 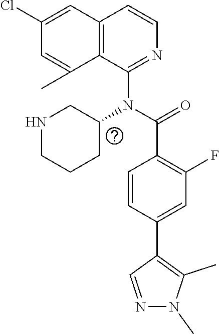 [SMILES: Cc1cc(Cl)cc2ccnc(N(C(=O)c3ccc(-c4cnn(C)c4C)cc3F)[C@@H]3CCCNC3)c12] |
| 815 | (R)-N-(6-chloro-8-methylisoquinolin-1- yl)-2-fluoro-4-(5-methyl-1,3,4-thiadiazol- 2-yl)-N-(piperidin-3-yl)benzamide | 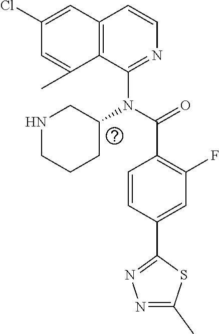 [SMILES: Cc1nnc(-c2ccc(C(=O)N(c3nccc4cc(Cl)cc(C)c34)[C@@H]3CCCNC3)c(F)c2)s1] |
| 816 | (R)-2-chloro-N-(6-chloro-8- methylisoquinolin-1-yl)-4-(1-methyl-1H- 1,2,3-triazol-4-yl)-N-(piperidin-3- yl)benzamide | 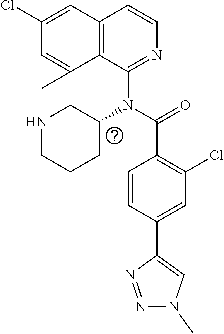 [SMILES: Cc1cc(Cl)cc2ccnc(N(C(=O)c3ccc(-c4cn(C)nn4)cc3Cl)[C@@H]3CCCNC3)c12] |
| 817 | (R)-N-(2-(5-amino-5-oxopent-1-yn-1- yl)thieno[3,2-c]pyridin-4-yl)-2-fluoro-4-(5- methyl-1,3,4-thiadiazol-2-yl)-N-(piperidin- 3-yl)benzamide | 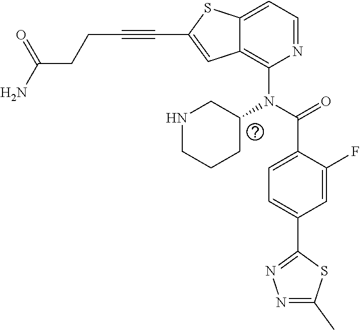 [SMILES: Cc1nnc(-c2ccc(C(=O)N(c3nccc4sc(C#CCCC(N)=O)cc34)[C@@H]3CCCNC3)c(F)c2)s1] |
| 818 | (R)-2-fluoro-N-(2-(4-hydroxybut-1-yn-1- yl)thieno[3,2-c]pyridin-4-yl)-4-(5-methyl- 1,3,4-thiadiazol-2-yl)-N-(piperidin-3- yl)benzamide | 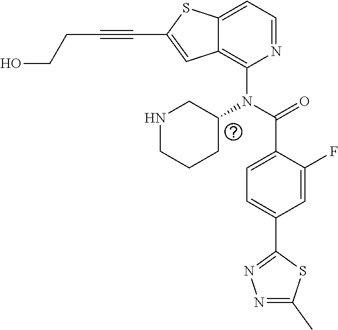 [SMILES: Cc1nnc(-c2ccc(C(=O)N(c3nccc4sc(C#CCCO)cc34)[C@@H]3CCCNC3)c(F)c2)s1] |
| 819 | (R)-N-(2-(4-carbamoylphenyl)thieno[3,2- c]pyridin-4-yl)-4-(1,5-dimethyl-1H-1,2,3- triazol-4-yl)-2-fluoro-N-(piperidin-3- yl)benzamide | 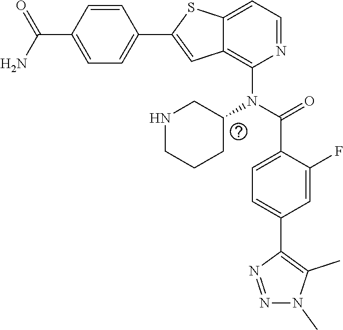 [SMILES: Cc1c(-c2ccc(C(=O)N(c3nccc4sc(-c5ccc(C(N)=O)cc5)cc34)[C@@H]3CCCNC3)c(F)c2)nnn1C] |
| 820 | (R)-4-(3H-[1,2,3]triazolo[4,5-b]pyridin-3- yl)-N-(2,3-dimethylthieno[3,2-c]pyridin-4- yl)-2-fluoro-N-(piperidin-3-yl)benzamide | 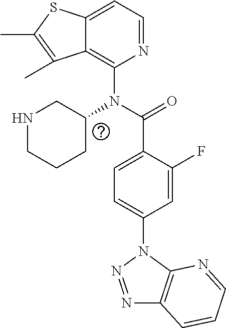 [SMILES: Cc1sc2ccnc(N(C(=O)c3ccc(-n4nnc5cccnc54)cc3F)[C@@H]3CCCNC3)c2c1C] |
| 821 | (R)-4-(1-ethyl-1H-1,2,3-triazol-4-yl)-2- fluoro-N-(8-methylisoquinolin-1-yl)-N- (piperidin-3-yl)benzamide | 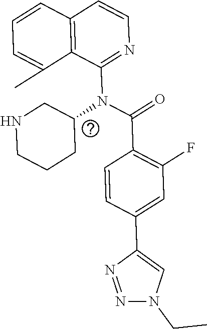 [SMILES: CCn1cc(-c2ccc(C(=O)N(c3nccc4cccc(C)c34)[C@@H]3CCCNC3)c(F)c2)nn1] |
| 822 | (R)-2-chloro-4-(1-methyl-1H-1,2,3-triazol- 4-yl)-N-(8-methylisoquinolin-1-yl)-N- (piperidin-3-yl)benzamide | 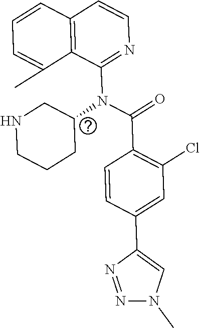 [SMILES: Cc1cccc2ccnc(N(C(=O)c3ccc(-c4cn(C)nn4)cc3Cl)[C@@H]3CCCNC3)c12] |
| 823 | (R)-2-fluoro-N-(8-methylisoquinolin-1-yl)- N-(piperidin-3-yl)-4-(thieno[2,3- d]pyrimidin-2-ylamino)benzamide | 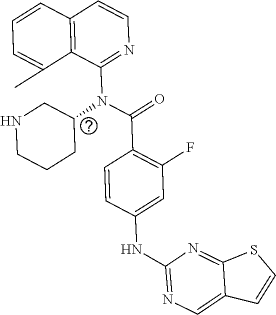 [SMILES: Cc1cccc2ccnc(N(C(=O)c3ccc(Nc4ncc5ccsc5n4)cc3F)[C@@H]3CCCNC3)c12] |
| 824 | (R)-2-fluoro-N-(8-methylisoquinolin-1-yl)- N-(piperidin-3-yl)-4-(quinazolin-2- ylamino)benzamide | 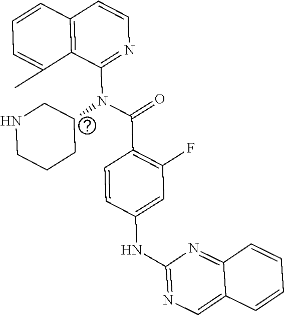 [SMILES: Cc1cccc2ccnc(N(C(=O)c3ccc(Nc4ncc5ccccc5n4)cc3F)[C@@H]3CCCNC3)c12] |
| 825 | (R)-N-(2-(4-carbamoylphenyl)thieno[3,2- c]pyridin-4-yl)-2-fluoro-N-(piperidin-3- yl)-4-(4,5,6,7-tetrahydropyrazolo[1,5- a]pyridin-3-yl)benzamide | 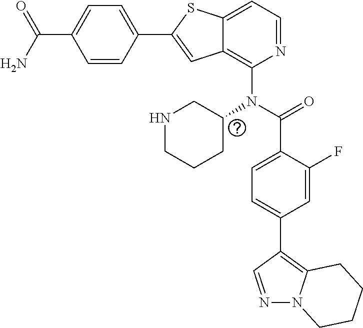 [SMILES: NC(=O)c1ccc(-c2cc3c(N(C(=O)c4ccc(-c5cnn6c5CCCC6)cc4F)[C@@H]4CCCNC4)nccc3s2)cc1] |
| 826 | (R)-4-(1H-benzo[d]imidazol-1-yl)-N-(2-(4- carbamoylphenyl)thieno[3,2-c]pyridin-4- yl)-2-fluoro-N-(piperidin-3-yl)benzamide | 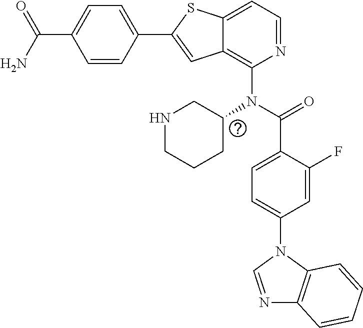 [SMILES: NC(=O)c1ccc(-c2cc3c(N(C(=O)c4ccc(-n5cnc6ccccc65)cc4F)[C@@H]4CCCNC4)nccc3s2)cc1] |
| 827 | (R)-N-(2-(4-carbamoylphenyl)thieno[3,2- c]pyridin-4-yl)-2-fluoro-4-(5-methyl-1,3,4- thiadiazol-2-yl)-N-(piperidin-3- yl)benzamide | 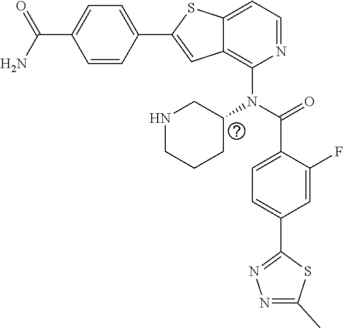 [SMILES: Cc1nnc(-c2ccc(C(=O)N(c3nccc4sc(-c5ccc(C(N)=O)cc5)cc34)[C@@H]3CCCNC3)c(F)c2)s1] |
| 828 | (R)-2-fluoro-4-(1-methyl-1H-1,2,3-triazol- 4-yl)-N-(piperidin-3-yl)-N-(6-(pyridin-3- ylmethyl)isoquinolin-1-yl)benzamide | 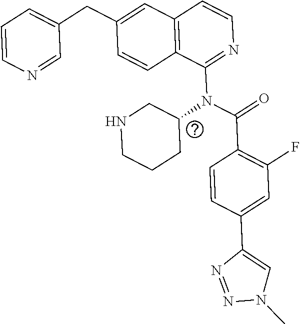 [SMILES: Cn1cc(-c2ccc(C(=O)N(c3nccc4cc(Cc5cccnc5)ccc34)[C@@H]3CCCNC3)c(F)c2)nn1] |
| 829 | (R)-4-(1,5-dimethyl-1H-1,2,3-triazol-4-yl)- 2-fluoro-N-(6-fluoro-8-methylisoquinolin- 1-yl)-N-(piperidin-3-yl)benzamide | 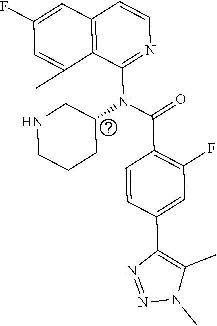 [SMILES: Cc1cc(F)cc2ccnc(N(C(=O)c3ccc(-c4nnn(C)c4C)cc3F)[C@@H]3CCCNC3)c12] |
| 830 | (R)-4-(1,5-dimethyl-1H-1,2,3-triazol-4-yl)- 2-fluoro-N-(8-methylisoquinolin-1-yl)-N- (piperidin-3-yl)benzamide | 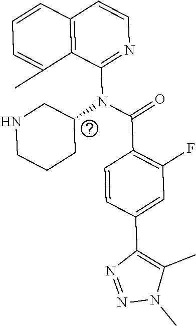 [SMILES: Cc1cccc2ccnc(N(C(=O)c3ccc(-c4nnn(C)c4C)cc3F)[C@@H]3CCCNC3)c12] |
| 831 | (R)-N-(2,3-dimethylthieno[3,2-c]pyridin-4- yl)-2-fluoro-4-(1-methyl-1H-1,2,3-triazol- 4-yl)-N-(piperidin-3-yl)benzamide | 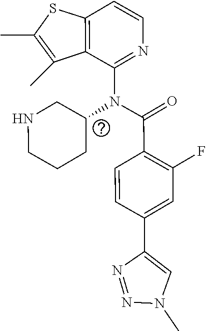 [SMILES: Cc1sc2ccnc(N(C(=O)c3ccc(-c4cn(C)nn4)cc3F)[C@@H]3CCCNC3)c2c1C] |
| 832 | (R)-4-(5-ethyl-1,3,4-thiadiazol-2-yl)-2- fluoro-N-(6-fluoro-8-methylisoquinolin-1- yl)-N-(piperidin-3-yl)benzamide | 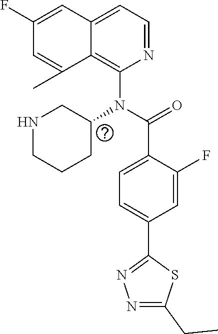 [SMILES: CCc1nnc(-c2ccc(C(=O)N(c3nccc4cc(F)cc(C)c34)[C@@H]3CCCNC3)c(F)c2)s1] |
| 833 | (R)-2-fluoro-N-(6-fluoro-8- methylisoquinolin-1-yl)-4-(5-methyl-1,3,4- thiadiazol-2-yl)-N-(piperidin-3- yl)benzamide | 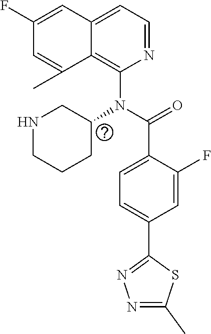 [SMILES: Cc1nnc(-c2ccc(C(=O)N(c3nccc4cc(F)cc(C)c34)[C@@H]3CCCNC3)c(F)c2)s1] |
| 834 | (R)-N-(6-(4- (aminomethyl)phenyl)isoquinolin-1-yl)-2- fluoro-4-(1-methyl-1H-1,2,3-triazol-4-yl)- N-(piperidin-3-yl)benzamide | 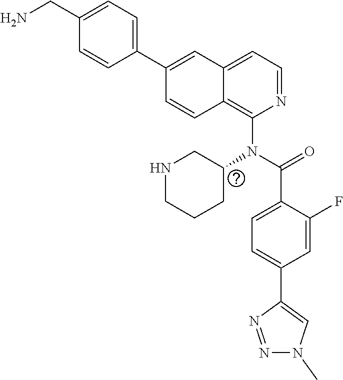 [SMILES: Cn1cc(-c2ccc(C(=O)N(c3nccc4cc(-c5ccc(CN)cc5)ccc34)[C@@H]3CCCNC3)c(F)c2)nn1] |
| 836 | (R)-4-(5-cyclopropyl-1,3,4-thiadiazol-2- yl)-2-fluoro-N-(8-methylisoquinolin-1-yl)- N-(piperidin-3-yl)benzamide | 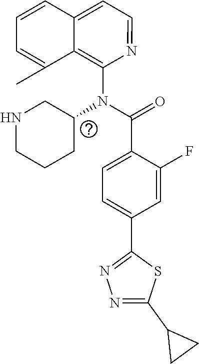 [SMILES: Cc1cccc2ccnc(N(C(=O)c3ccc(-c4nnc(C5CC5)s4)cc3F)[C@@H]3CCCNC3)c12] |
| 837 | (R)-4-(5-ethyl-1,3,4-thiadiazol-2-yl)-2- fluoro-N-(8-methylisoquinolin-1-yl)-N- (piperidin-3-yl)benzamide | 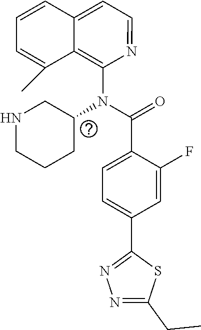 [SMILES: CCc1nnc(-c2ccc(C(=O)N(c3nccc4cccc(C)c34)[C@@H]3CCCNC3)c(F)c2)s1] |
| 839 | (R)-2-fluoro-N-(2-(5-hydroxypent-1-yn-1- yl)thieno[3,2-c]pyridin-4-yl)-N-(piperidin- 3-yl)-4-(4,5,6,7-tetrahydropyrazolo[1,5- alpyridin-3-yl)benzamide | 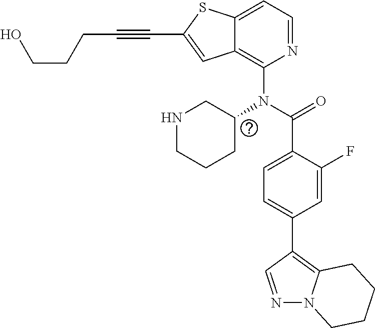 [SMILES: O=C(c1ccc(-c2cnn3c2CCCC3)cc1F)N(c1nccc2sc(C#CCCCO)cc12)[C@@H]1CCCNC1] |
| 840 | (R)-N-(2-(4-carbamoylphenyl)thieno[3,2- c]pyridin-4-yl)-2-fluoro-4-(1-methyl-1H- 1,2,3-triazol-4-yl)-N-(piperidin-3- yl)benzamide | 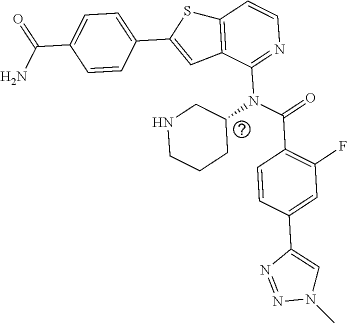 [SMILES: Cn1cc(-c2ccc(C(=O)N(c3nccc4sc(-c5ccc(C(N)=O)cc5)cc34)[C@@H]3CCCNC3)c(F)c2)nn1] |
| 841 | (R)-2-fluoro-4-(1-methyl-1H-1,2,3-triazol- 4-yl)-N-(3-methylthieno[3,2-c]pyridin-4- yl)-N-(piperidin-3-yl)benzamide | 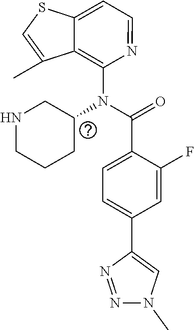 [SMILES: Cc1csc2ccnc(N(C(=O)c3ccc(-c4cn(C)nn4)cc3F)[C@@H]3CCCNC3)c12] |
| 842 | (R)-2-fluoro-4-(1-methyl-1H-1,2,3-triazol- 4-yl)-N-(piperidin-3-yl)-N-(3-(prop-1-en- 2-yl)thieno[3,2-c]pyridin-4-yl)benzamide | 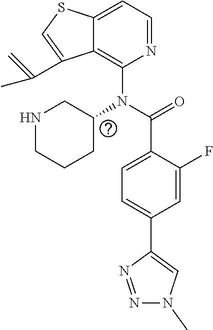 [SMILES: C=C(C)c1csc2ccnc(N(C(=O)c3ccc(-c4cn(C)nn4)cc3F)[C@@H]3CCCNC3)c12] |
| 843 | (R)-N-(8-methylisoquinolin-1-yl)-N- (piperidin-3-yl)-6-(pyridin-2- ylamino)nicotinamide | 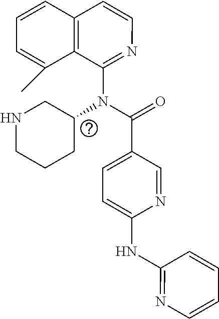 [SMILES: Cc1cccc2ccnc(N(C(=O)c3ccc(Nc4ccccn4)nc3)[C@@H]3CCCNC3)c12] |
| 844 | (R)-4-(3H-[1,2,3]triazolo[4,5-b]pyridin-3- yl)-2-fluoro-N-(2-(5-hydroxypent-1-yn-1- yl)thieno[3,2-c]pyridin-4-yl)-N-(piperidin- 3-yl)benzamide | 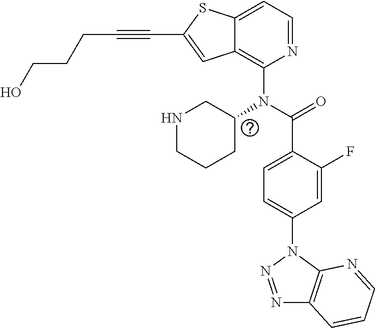 [SMILES: O=C(c1ccc(-n2nnc3cccnc32)cc1F)N(c1nccc2sc(C#CCCCO)cc12)[C@@H]1CCCNC1] |
| 845 | 2-fluoro-N-[8-methyl-6-(1-methylpyrazol- 4-yl)-1-isoquinolyl]-4-(1-methyltriazol-4- yl)-N-[(3R)-3-piperidyl]benzamide | 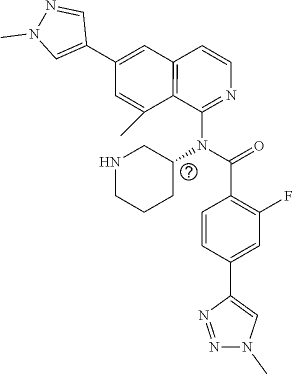 [SMILES: Cc1cc(-c2cnn(C)c2)cc2ccnc(N(C(=O)c3ccc(-c4cn(C)nn4)cc3F)[C@@H]3CCCNC3)c12] |
| 846 | (R)-N-(8-methylisoquinolin-1-yl)-N- (piperidin-3-yl)-6-(pyrimidin-2- ylamino)nicotinamide | 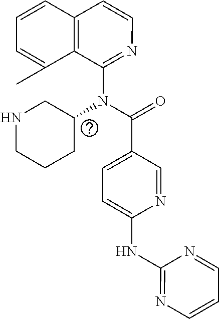 [SMILES: Cc1cccc2ccnc(N(C(=O)c3ccc(Nc4ncccn4)nc3)[C@@H]3CCCNC3)c12] |
| 847 | (R)-3-chloro-N-(8-methylisoquinolin-1- yl)-N-(piperidin-3-yl)-4-(pyrimidin-2- ylamino)benzamide | 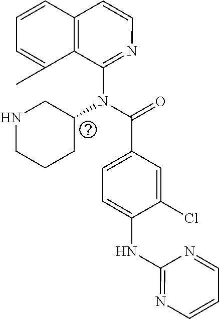 [SMILES: Cc1cccc2ccnc(N(C(=O)c3ccc(Nc4ncccn4)c(Cl)c3)[C@@H]3CCCNC3)c12] |
| 848 | (R)-4-((4-aminopyrimidin-2-yl)amino)-2- fluoro-N-(8-methylisoquinolin-1-yl)-N- (piperidin-3-yl)benzamide | 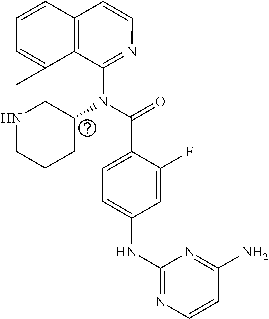 [SMILES: Cc1cccc2ccnc(N(C(=O)c3ccc(Nc4nccc(N)n4)cc3F)[C@@H]3CCCNC3)c12] |
| 849 | (R)-4-(3H-[1,2,3]triazolo[4,5-b]pyridin-3- yl)-2-fluoro-N-(2,6-naphthyridin-1-yl)-N- (piperidin-3-yl)benzamide | 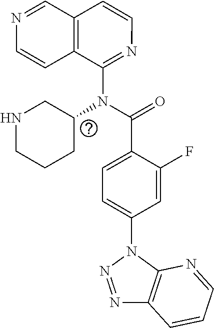 [SMILES: O=C(c1ccc(-n2nnc3cccnc32)cc1F)N(c1nccc2cnccc12)[C@@H]1CCCNC1] |
| 850 | (R)-2-fluoro-N-(6-fluoro-8- methylisoquinolin-1-yl)-4-(1-(2- methoxyethyl)-1H-1,2,3-triazol-4-yl)-N- (piperidin-3-yl)benzamide | 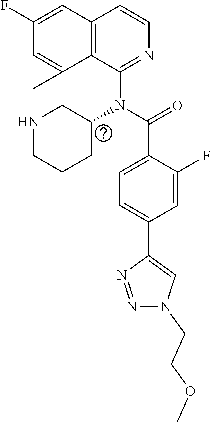 [SMILES: COCCn1cc(-c2ccc(C(=O)N(c3nccc4cc(F)cc(C)c34)[C@@H]3CCCNC3)c(F)c2)nn1] |
| 851 | (R)-2-fluoro-4-(5-(methoxymethyl)-1- methyl-1H-1,2,3-triazol-4-yl)-N-(3- methylthieno[3,2-c]pyridin-4-yl)-N- (piperidin-3-yl)benzamide | 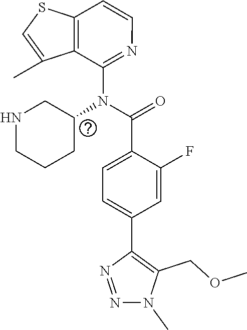 |
| 852 | (R)-2-fluoro-N-(6-fluoro-8- methylisoquinolin-1-yl)-4-(5-(2- methoxyethyl)-1,3,4-thiadiazol-2-yl)-N- (piperidin-3-yl)benzamide | 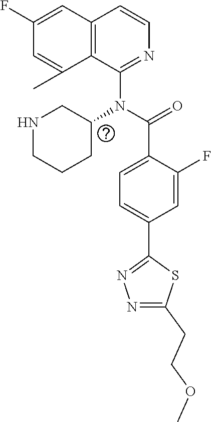 [SMILES: COCCc1nnc(-c2ccc(C(=O)N(c3nccc4cc(F)cc(C)c34)[C@@H]3CCCNC3)c(F)c2)s1] |
| 853 | (R)-2-fluoro-N-(6-fluoro-8- methylisoquinolin-1-yl)-4-(5- (methoxymethyl)-1,3,4-thiadiazol-2-yl)-N- (piperidin-3-yl)benzamide | 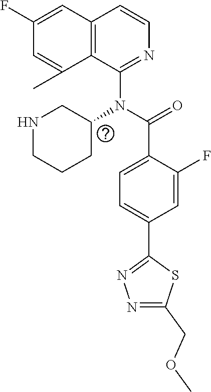 [SMILES: COCc1nnc(-c2ccc(C(=O)N(c3nccc4cc(F)cc(C)c34)[C@@H]3CCCNC3)c(F)c2)s1] |
| 854 | (R)-4-(5-(ethoxymethyl)-1,3,4-thiadiazol- 2-yl)-2-fluoro-N-(6-fluoro-8- methylisoquinolin-1-yl)-N-(piperidin-3- yl)benzamide | 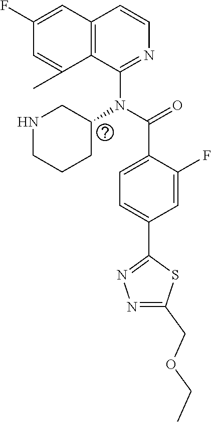 [SMILES: CCOCc1nnc(-c2ccc(C(=O)N(c3nccc4cc(F)cc(C)c34)[C@@H]3CCCNC3)c(F)c2)s1] |
| 855 | (R)-N-(5-chloro-8-methylisoquinolin-1- yl)-4-(1-methyl-1H-1,2,3-triazol-4-yl)-N- (piperidin-3-yl)benzamide | 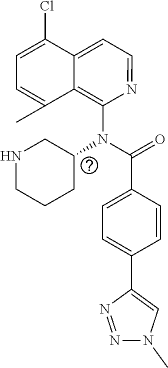 [SMILES: Cc1ccc(Cl)c2ccnc(N(C(=O)c3ccc(-c4cn(C)nn4)cc3)[C@@H]3CCCNC3)c12] |
| 856 | (R)-N-(5-chloro-8-methylisoquinolin-1- yl)-4-(5-methyl-1,3,4-thiadiazol-2-yl)-N- (piperidin-3-yl)benzamide | 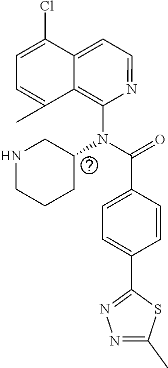 [SMILES: Cc1nnc(-c2ccc(C(=O)N(c3nccc4c(Cl)ccc(C)c34)[C@@H]3CCCNC3)cc2)s1] |
| 857 | (R)-N-(5-chloro-8-methylisoquinolin-1- yl)-5-(5-methyl-1,3,4-thiadiazol-2-yl)-N- (piperidin-3-yl)picolinamide | 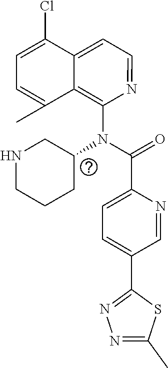 [SMILES: Cc1nnc(-c2ccc(C(=O)N(c3nccc4c(Cl)ccc(C)c34)[C@@H]3CCCNC3)nc2)s1] |
| 858 | (R)-N-(6-methoxy-8-methylisoquinolin-1- yl)-4-(1-methyl-1H-1,2,3-triazol-4-yl)-N- (piperidin-3-yl)benzamide | 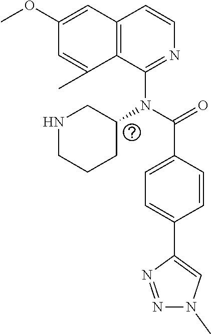 |
| 859 | (R)-4-(1-ethyl-1H-1,2,3-triazol-4-yl)-N-(3- methylthieno[3,2-c]pyridin-4-yl)-N- (piperidin-3-yl)benzamide | 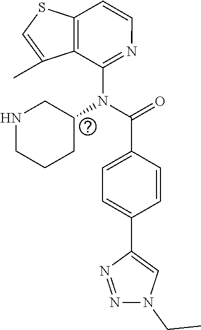 [SMILES: CCn1cc(-c2ccc(C(=O)N(c3nccc4scc(C)c34)[C@@H]3CCCNC3)cc2)nn1] |
| 860 | (R)-2-fluoro-N-(6-methoxy-8- methylisoquinolin-1-yl)-4-(5-methyl-1,3,4- thiadiazol-2-yl)-N-(piperidin-3- yl)benzamide | 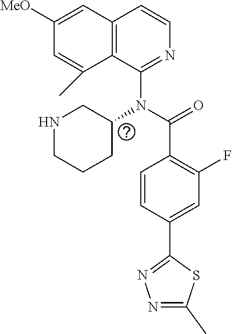 [SMILES: COc1cc(C)c2c(N(C(=O)c3ccc(-c4nnc(C)s4)cc3F)[C@@H]3CCCNC3)nccc2c1] |
| 861 | (R)-N-(5,8-dimethylisoquinolin-1-yl)-4-(1- methyl-1H-1,2,3-triazol-4-yl)-N- (piperidin-3-yl)benzamide | 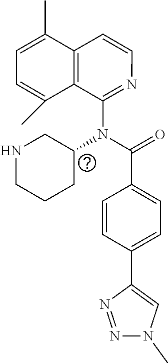 [SMILES: Cc1ccc(C)c2c(N(C(=O)c3ccc(-c4cn(C)nn4)cc3)[C@@H]3CCCNC3)nccc12] |
| 862 | (R)-N-(5,6-difluoro-8-methylisoquinolin-1- yl)-4-(1-methyl-1H-1,2,3-triazol-4-yl)-N- (piperidin-3-yl)benzamide | 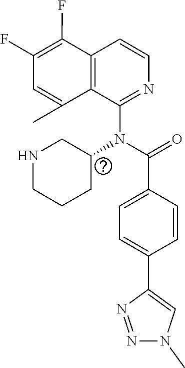 [SMILES: Cc1cc(F)c(F)c2ccnc(N(C(=O)c3ccc(-c4cn(C)nn4)cc3)[C@@H]3CCCNC3)c12] |
| 863 | (R)-4-(1-ethyl-1H-1,2,3-triazol-4-yl)-2- fluoro-N-(6-methoxy-8-methylisoquinolin- 1-yl)-N-(piperidin-3-yl)benzamide | 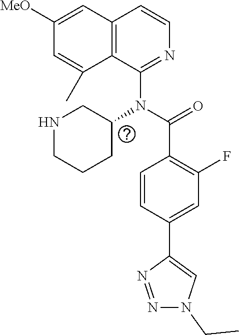 [SMILES: CCn1cc(-c2ccc(C(=O)N(c3nccc4cc(OC)cc(C)c34)[C@@H]3CCCNC3)c(F)c2)nn1] |
| 864 | (R)-N-(6-methoxy-8-methylisoquinolin-1- yl)-5-(5-methyl-1,3,4-thiadiazol-2-yl)-N- (piperidin-3-yl)picolinamide | 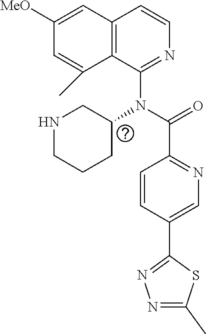 [SMILES: COc1cc(C)c2c(N(C(=O)c3ccc(-c4nnc(C)s4)cn3)[C@@H]3CCCNC3)nccc2c1] |
| 865 | (R)-N-(6-ethoxy-8-methylisoquinolin-1- yl)-5-(5-methyl-1,3,4-thiadiazol-2-yl)-N- (piperidin-3-yl)picolinamide | 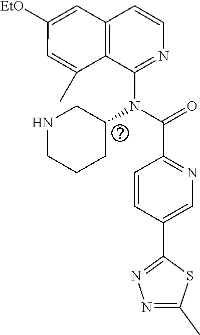 [SMILES: CCOc1cc(C)c2c(N(C(=O)c3ccc(-c4nnc(C)s4)cn3)[C@@H]3CCCNC3)nccc2c1] |
| 866 | (R)-N-(6-methoxy-8-methylisoquinolin-1- yl)-4-(5-methyl-1,3,4-thiadiazol-2-yl)-N- (piperidin-3-yl)benzamide | 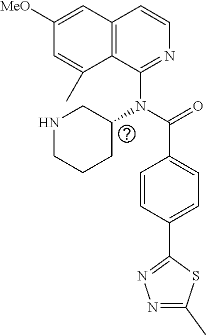 [SMILES: COc1cc(C)c2c(N(C(=O)c3ccc(-c4nnc(C)s4)cc3)[C@@H]3CCCNC3)nccc2c1] |
| 867 | (R)-N-(5-fluoro-6-methoxyisoquinolin-1- yl)-5-(5-methyl-1,3,4-thiadiazol-2-yl)-N- (piperidin-3-yl)picolinamide | 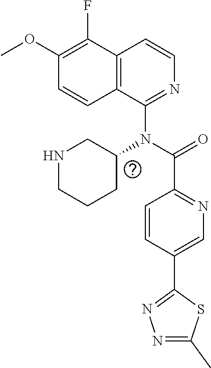 |
| 868 | (R)-N-(5-fluoro-6-methoxyisoquinolin-1- yl)-4-(5-methyl-1,3,4-thiadiazol-2-yl)-N- (piperidin-3-yl)benzamide | 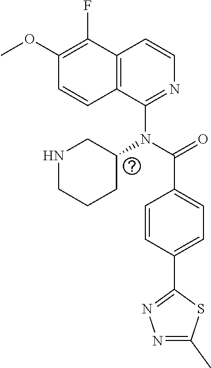 |
| 869 | (R)-N-(5-chloro-6-methoxyisoquinolin-1- yl)-4-(1-methyl-1H-1,2,3-triazol-4-yl)-N- (piperidin-3-yl)benzamide | 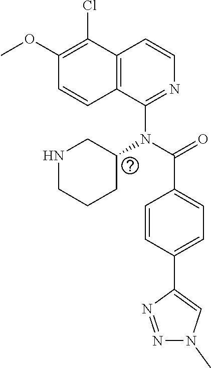 |
| 870 | (R)-N-(5-methoxy-8-methylisoquinolin-1- yl)-5-(5-methyl-1,3,4-thiadiazol-2-yl)-N- (piperidin-3-yl)picolinamide | 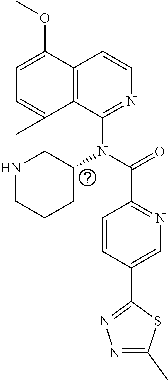 [SMILES: COc1ccc(C)c2c(N(C(=O)c3ccc(-c4nnc(C)s4)cn3)[C@@H]3CCCNC3)nccc12] |
| 871 | (R)-N-(5-methoxy-8-methylisoquinolin-1- yl)-4-(5-methyl-1,3,4-thiadiazol-2-yl)-N- (piperidin-3-yl)benzamide | 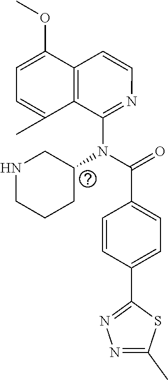 [SMILES: COc1ccc(C)c2c(N(C(=O)c3ccc(-c4nnc(C)s4)cc3)[C@@H]3CCCNC3)nccc12] |
| 872 | (R)-N-(5-methoxy-8-methylisoquinolin-1- yl)-4-(1-methyl-1H-1,2,3-triazol-4-yl)-N- (piperidin-3-yl)benzamide | 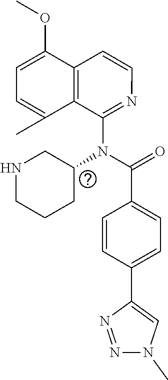 [SMILES: COc1ccc(C)c2c(N(C(=O)c3ccc(-c4cn(C)nn4)cc3)[C@@H]3CCCNC3)nccc12] |
| 873 | (R)-N-(8-fluoro-5-methoxyisoquinolin-1- yl)-4-(1-methyl-1H-1,2,3-triazol-4-yl)-N- (piperidin-3-yl)benzamide | 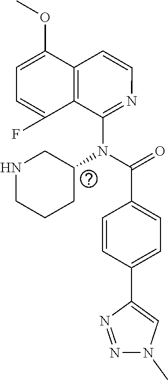 [SMILES: COc1ccc(F)c2c(N(C(=O)c3ccc(-c4cn(C)nn4)cc3)[C@@H]3CCCNC3)nccc12] |
| 874 | (R)-2-fluoro-N-(6-fluoro-5- methoxyisoquinolin-1-yl)-4-(1-methyl-1H- 1,2,3-triazol-4-yl)-N-(piperidin-3- yl)benzamide | 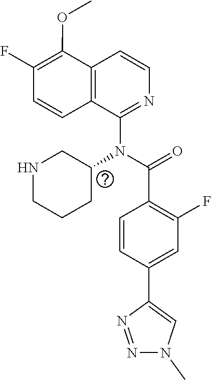 [SMILES: COc1c(F)ccc2c(N(C(=O)c3ccc(-c4cn(C)nn4)cc3F)[C@@H]3CCCNC3)nccc12] |
| 875 | (R)-4-(5-(methoxymethyl)-1,3,4- thiadiazol-2-yl)-N-(3-methylthieno[3,2- c]pyridin-4-yl)-N-(piperidin-3- yl)benzamide | 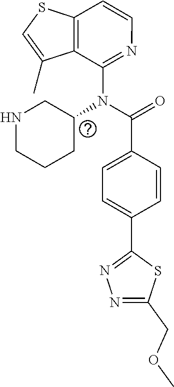 [SMILES: COCc1nnc(-c2ccc(C(=O)N(c3nccc4scc(C)c34)[C@@H]3CCCNC3)cc2)s1] |
| 876 | (R)-4-(5-(3-methoxypropyl)-1,3,4- thiadiazol-2-yl)-N-(3-methylthieno[3,2- c]pyridin-4-yl)-N-(piperidin-3- yl)benzamide | 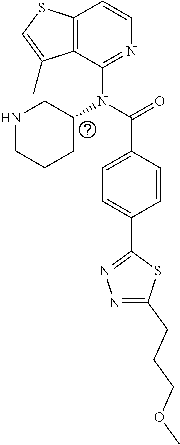 [SMILES: COCCCc1nnc(-c2ccc(C(=O)N(c3nccc4scc(C)c34)[C@@H]3CCCNC3)cc2)s1] |
| 877 | (R)-N-(5,8-dimethylisoquinolin-1-yl)-5-(5- methyl-1,3,4-thiadiazol-2-yl)-N-(piperidin- 3-yl)picolinamide | 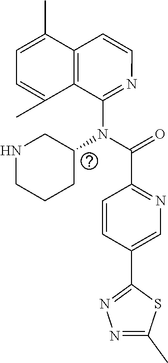 [SMILES: Cc1nnc(-c2ccc(C(=O)N(c3nccc4c(C)ccc(C)c34)[C@@H]3CCCNC3)nc2)s1] |
| 878 | (R)-N-(5,8-dimethylisoquinolin-1-yl)-4-(5- methyl-1,3,4-thiadiazol-2-yl)-N-(piperidin- 3-yl)benzamide | 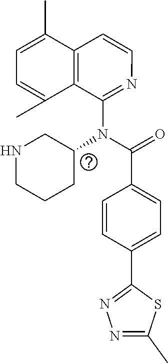 [SMILES: Cc1nnc(-c2ccc(C(=O)N(c3nccc4c(C)ccc(C)c34)[C@@H]3CCCNC3)cc2)s1] |
| 879 | (R)-4-(5-(2-cyanoethyl)-1,3,4-thiadiazol-2- yl)-2-fluoro-N-(8-methylisoquinolin-1-yl)- N-(piperidin-3-yl)benzamide | 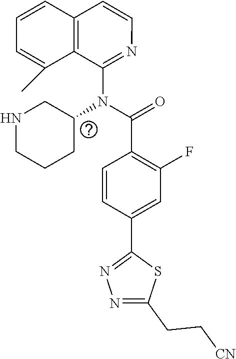 [SMILES: Cc1cccc2ccnc(N(C(=O)c3ccc(-c4nnc(CCC#N)s4)cc3F)[C@@H]3CCCNC3)c12] |
| 880 | (R)-4-(5-(2-cyanoethyl)-1,3,4-thiadiazol-2- yl)-N-(8-methylisoquinolin-1-yl)-N- (piperidin-3-yl)benzamide | 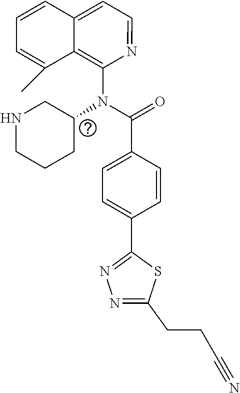 [SMILES: Cc1cccc2ccnc(N(C(=O)c3ccc(-c4nnc(CCC#N)s4)cc3)[C@@H]3CCCNC3)c12] |
| 881 | (R)-4-(1-(2-cyanoethyl)-1H-1,2,3-triazol- 4-yl)-2-fluoro-N-(3-methylthieno[3,2- c]pyridin-4-yl)-N-(piperidin-3- yl)benzamide | 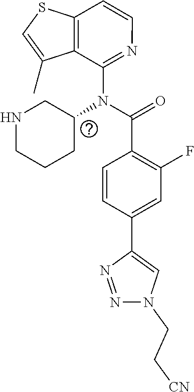 [SMILES: Cc1csc2ccnc(N(C(=O)c3ccc(-c4cn(CCC#N)nn4)cc3F)[C@@H]3CCCNC3)c12] |
| 882 | (R)-4-(5-(2-cyanoethyl)-1,3,4-thiadiazol-2- yl)-N-(3-methylthieno[3,2-c]pyridin-4-yl)- N-(piperidin-3-yl)benzamide | 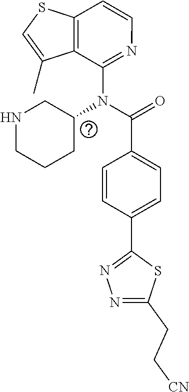 [SMILES: Cc1csc2ccnc(N(C(=O)c3ccc(-c4nnc(CCC#N)s4)cc3)[C@@H]3CCCNC3)c12] |
| 883 | (R)-4-(5-(2-cyanoethyl)-1,3,4-thiadiazol-2- yl)-2-fluoro-N-(3-methylthieno[3,2- c]pyridin-4-yl)-N-(piperidin-3- yl)benzamide | 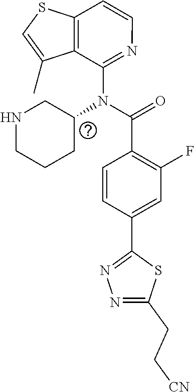 [SMILES: Cc1csc2ccnc(N(C(=O)c3ccc(-c4nnc(CCC#N)s4)cc3F)[C@@H]3CCCNC3)c12] |
| 884 | (R)-N-(6-chloro-8-methylisoquinolin-1- yl)-6-(1-methyl-1H-1,2,3-triazol-4-yl)-N- (piperidin-3-yl)nicotinamide | 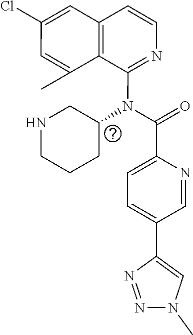 [SMILES: Cc1cc(Cl)cc2ccnc(N(C(=O)c3ccc(-c4cn(C)nn4)cn3)[C@@H]3CCCNC3)c12] |
| 885 | (R)-6-(1-ethyl-1H-1,2,3-triazol-4-yl)-N-(3- methylthieno[3,2-c]pyridin-4-yl)-N- (piperidin-3-yl)nicotinamide | 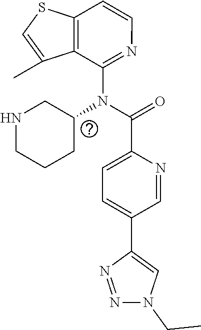 [SMILES: CCn1cc(-c2ccc(C(=O)N(c3nccc4scc(C)c34)[C@@H]3CCCNC3)nc2)nn1] |
| 886 | (R)-2-fluoro-4-(5-methyl-1,3,4-thiadiazol- 2-yl)-N-(piperidin-3-yl)-N-(3- vinylthieno[3,2-c]pyridin-4-yl)benzamide | 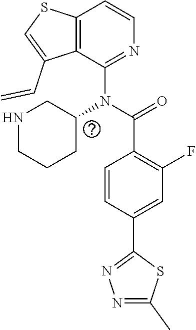 [SMILES: C=Cc1csc2ccnc(N(C(=O)c3ccc(-c4nnc(C)s4)cc3F)[C@@H]3CCCNC3)c12] |
| 887 | (R)-2-fluoro-4-(1-methyl-1H-1,2,3-triazol- 4-yl)-N-(piperidin-3-yl)-N-(4-(prop-1-en- 2-yl)pyridin-2-yl)benzamide | 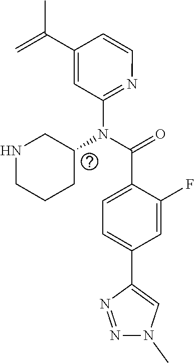 [SMILES: C=C(C)c1ccnc(N(C(=O)c2ccc(-c3cn(C)nn3)cc2F)[C@@H]2CCCNC2)c1] |
| 888 | (R)-4-(1-(2-cyanoethyl)-1H-1,2,3-triazol- 4-yl)-2-fluoro-N-(8-methylisoquinolin-1- yl)-N-(piperidin-3-yl)benzamide | 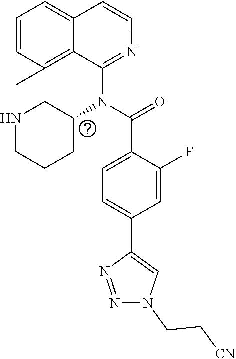 [SMILES: Cc1cccc2ccnc(N(C(=O)c3ccc(-c4cn(CCC#N)nn4)cc3F)[C@@H]3CCCNC3)c12] |
| 889 | (R)-4-(1-ethyl-1H-1,2,3-triazol-4-yl)-2- fluoro-N-(6-(methoxy-d3)-8- methylisoquinolin-1-yl)-N-(piperidin-3- yl)benzamide | 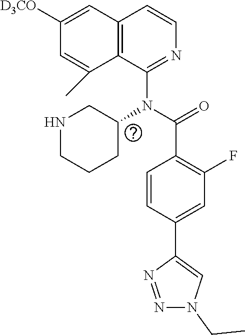 |
| 890 | (R)-2-fluoro-4-(1-(methyl-d3)-1H-1,2,3- triazol-4-yl)-N-(8-methylisoquinolin-1-yl)- N-(piperidin-3-yl)benzamide | 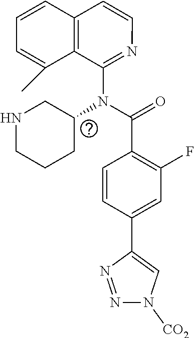 |
| 891 | (R)-2-fluoro-N-(6-fluoro-8- methylisoquinolin-1-yl)-4-(1-(methyl-d3)- 1H-1,2,3-triazol-4-yl)-N-(piperidin-3- yl)benzamide | 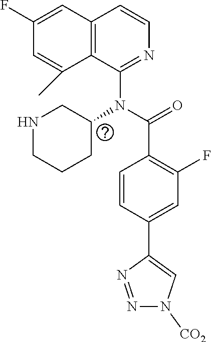 |
| 892 | (R)-N-(6-chloro-8-methylisoquinolin-1- yl)-4-(1-(methyl-d3)-1H-1,2,3-triazol-4- yl)-N-(piperidin-3-yl)benzamide | 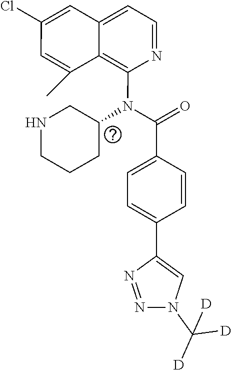 [SMILES: [2H]C([2H])([2H])n1cc(-c2ccc(C(=O)N(c3nccc4cc(Cl)cc(C)c34)[C@@H]3CCCNC3)cc2)nn1] |
| 893 | (R)-N-(6-chloro-8-methylisoquinolin-1- yl)-2-fluoro-4-(1-(methyl-d3)-1H-1,2,3- triazol-4-yl)-N-(piperidin-3-yl)benzamide | 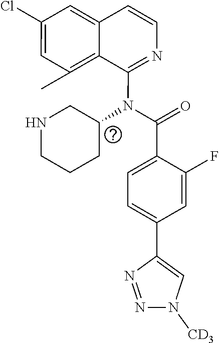 [SMILES: [2H]C([2H])([2H])n1cc(-c2ccc(C(=O)N(c3nccc4cc(Cl)cc(C)c34)[C@@H]3CCCNC3)c(F)c2)nn1] |
| 894 | (R)-4-(5-(methyl-d3)-1,3,4-thiadiazol-2- yl)-N-(3-methylthieno[3,2-c]pyridin-4-yl)- N-(piperidin-3-yl)benzamide | 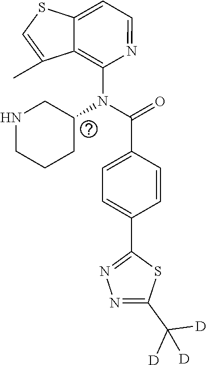 [SMILES: [2H]C([2H])([2H])c1nnc(-c2ccc(C(=O)N(c3nccc4scc(C)c34)[C@@H]3CCCNC3)cc2)s1] |
| 895 | (R)-N-(6-chloro-8-methylisoquinolin-1- yl)-6-(5-(methoxymethyl)-1,3,4-thiadiazol- 2-yl)-N-(piperidin-3-yl)nicotinamide | 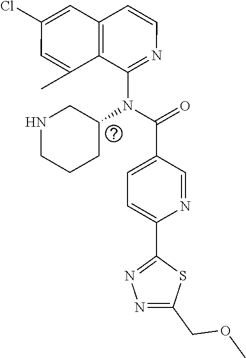 [SMILES: COCc1nnc(-c2ccc(C(=O)N(c3nccc4cc(Cl)cc(C)c34)[C@@H]3CCCNC3)cn2)s1] |
| 896 | (R)-N-(6,8-dimethylisoquinolin-1-yl)-6-(5- (methoxymethyl)-1,3,4-thiadiazol-2-yl)-N- (piperidin-3-yl)nicotinamide | 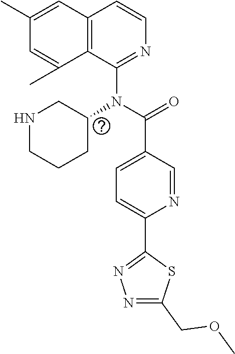 |
| 897 | (R)-N-(5,8-dimethylisoquinolin-1-yl)-6-(5- methyl-1,3,4-thiadiazol-2-yl)-N-(piperidin- 3-yl)nicotinamide | 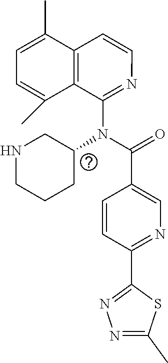 [SMILES: Cc1nnc(-c2ccc(C(=O)N(c3nccc4c(C)ccc(C)c34)[C@@H]3CCCNC3)cn2)s1] |
| 898 | (R)-N-(5-chloro-8-methylisoquinolin-1- yl)-6-(5-methyl-1,3,4-thiadiazol-2-yl)-N- (piperidin-3-yl)nicotinamide | 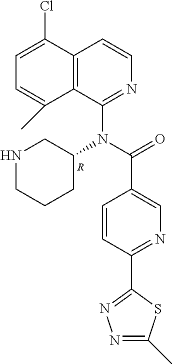 |
| 899 | (R)-2-fluoro-4-(5-(methyl-d3)-1,3,4- thiadiazol-2-yl)-N-(8-methylisoquinolin-1- yl)-N-(piperidin-3-yl)benzamide | 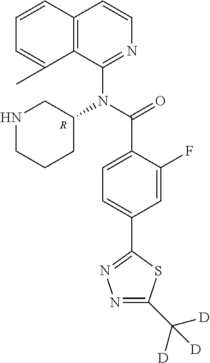 |
| 900 | (R)-6-(5-ethyl-1,3,4-thiadiazol-2-yl)-N-(8- methylisoquinolin-1-yl)-N-(piperidin-3- yl)nicotinamide | 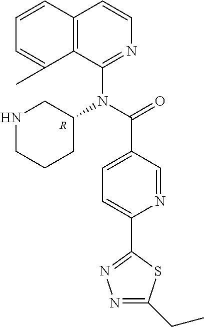 |
| 901 | (R)-N-(6-methoxy-8-methylisoquinolin-1- yl)-6-(5-methyl-1,3,4-thiadiazol-2-yl)-N- (piperidin-3-yl)nicotinamide | 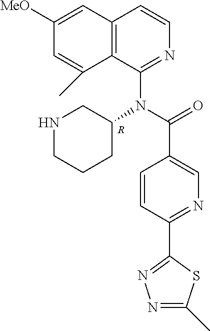 |
| 902 | (R)-N-(5-methoxy-8-methylisoquinolin-1- yl)-6-(5-methyl-1,3,4-thiadiazol-2-yl)-N- (piperidin-3-yl)nicotinamide | 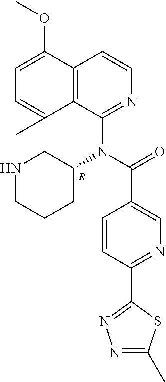 |
| 903 | (R)-N-(6,8-dimethylisoquinolin-1-yl)-6-(5- (2-methoxyethyl)-1,3,4-thiadiazol-2-yl)-N- (piperidin-3-yl)nicotinamide | 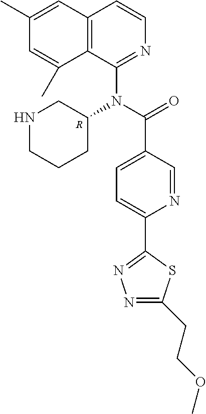 |
| 904 | (R)-N-(6,8-dimethylisoquinolin-1-yl)-6-(5- (3-methoxypropyl)-1,3,4-thiadiazol-2-yl)- N-(piperidin-3-yl)nicotinamide | 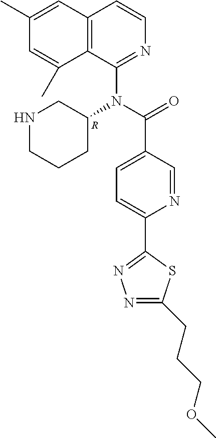 |
| 905 | (R)-N-(6-methoxy-8-methylisoquinolin-1- yl)-6-(5-(methyl-d3)-1,3,4-thiadiazol-2-yl)- N-(piperidin-3-yl)nicotinamide | 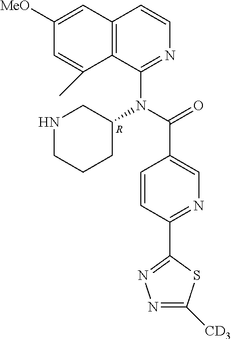 |
| 906 | (R)-N-(6,8-dimethylisoquinolin-1-yl)-2- fluoro-4-(5-(methyl-d3)-1,3,4-thiadiazol-2- yl)-N-(piperidin-3-yl)benzamide | 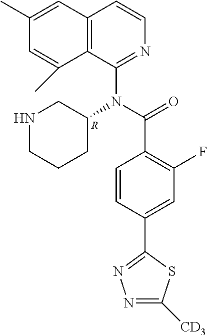 |
| 907 | (R)-N-(6,8-dimethylisoquinolin-1-yl)-4-(5- (methyl-d3)-1,3,4-thiadiazol-2-yl)-N- (piperidin-3-yl)benzamide | 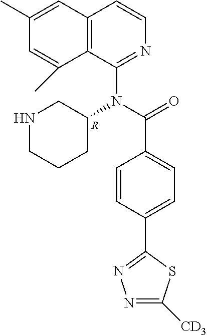 |
| 908 | (R)-N-(6-methoxy-8-methylisoquinolin-1- yl)-4-(5-(methyl-d3)-1,3,4-thiadiazol-2-yl)- N-(piperidin-3-yl)benzamide | 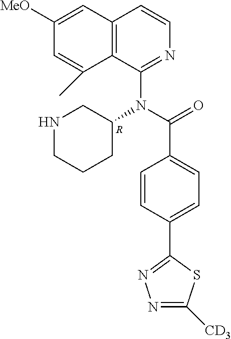 |
| 909 | (R)-N-(6-(methoxy-d3)-8- methylisoquinolin-1-yl)-6-(5-methyl-1,3,4- thiadiazol-2-yl)-N-(piperidin-3- yl)nicotinamide | 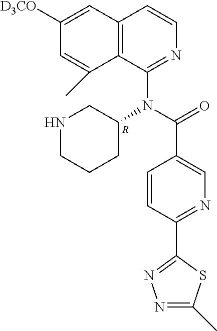 |
| 910 | (R)-6-(5-(2-methoxyethyl)-1,3,4- thiadiazol-2-yl)-N-(8-methylisoquinolin-1- yl)-N-(piperidin-3-yl)nicotinamide | 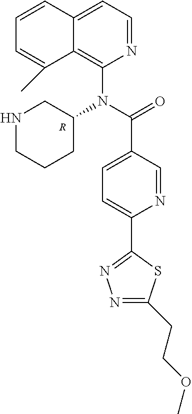 |
| 911 | (R)-N-(5-fluoro-6-methoxy-8- methylisoquinolin-1-yl)-6-(5-methyl-1,3,4- thiadiazol-2-yl)-N-(piperidin-3- yl)nicotinamide | 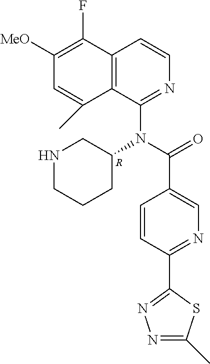 |
| 912 | (R)-6-(5-(2-cyanoethyl)-1,3,4-thiadiazol-2- yl)-N-(3-methylthieno[3,2-c]pyridin-4-yl)- N-(piperidin-3-yl)nicotinamide | 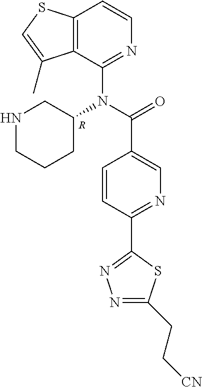 |
| 913 | (R)-6-(5-(2-methoxyethyl)-1,3,4- thiadiazol-2-yl)-N-(3-methylthieno[3,2- c]pyridin-4-yl)-N-(piperidin-3- yl)nicotinamide | 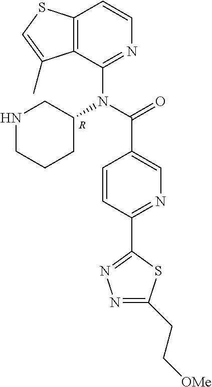 |
| 914 | (R)-N-(6-methoxy-8-methylisoquinolin-1- yl)-4-(3-methyl-1,2,4-oxadiazol-5-yl)-N- (piperidin-3-yl)benzamide | 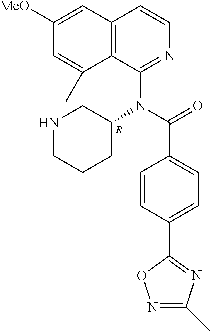 |
| 915 | (R)-N-(6,8-dimethylisoquinolin-1-yl)-4-(3- methyl-1,2,4-oxadiazol-5-yl)-N-(piperidin- 3-yl)benzamide | 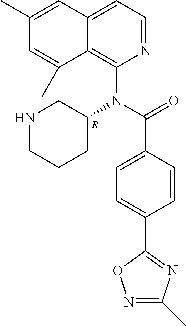 |
| 916 | (R)-N-(6,8-dimethylisoquinolin-1-yl)-6-(3- methyl-1,2,4-oxadiazol-5-yl)-N-(piperidin- 3-yl)nicotinamide | 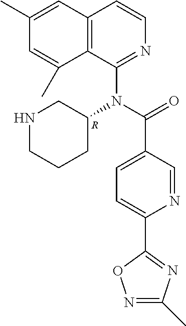 |
| 917 | (R)-N-(6-chloro-8-methylisoquinolin-1- yl)-6-(5-methyl-1,3,4-oxadiazol-2-yl)-N- (piperidin-3-yl)nicotinamide | 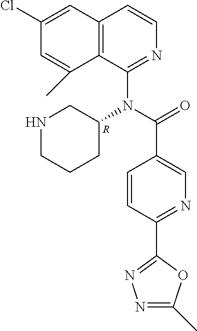 |
| 918 | (R)-N-(6-fluoro-8-methylisoquinolin-1-yl)- 6-(5-methyl-1,3,4-oxadiazol-2-yl)-N- (piperidin-3-yl)nicotinamide | 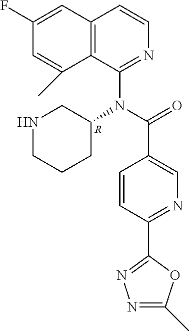 |
| 919 | (R)-6-(5-methyl-1,3,4-oxadiazol-2-yl)-N- (3-methylthieno[3,2-c]pyridin-4-yl)-N- (piperidin-3-yl)nicotinamide | 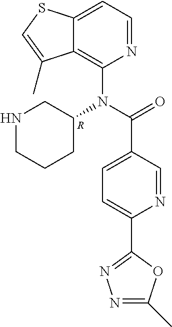 |
| 920 | (R)-N-(6-fluoro-8-methylisoquinolin-1-yl)- 6-(1-(2-methoxyethyl)-1H-1,2,3-triazol-4- yl)-N-(piperidin-3-yl)nicotinamide | 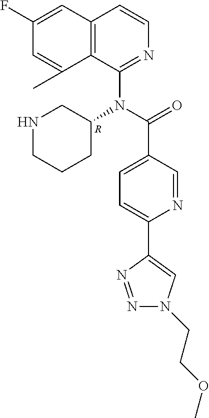 |
| 921 | (R)-N-(6-chloro-8-methylisoquinolin-1- yl)-6-(1-(2-methoxyethyl)-1H-1,2,3- triazol-4-yl)-N-(piperidin-3- yl)nicotinamide | 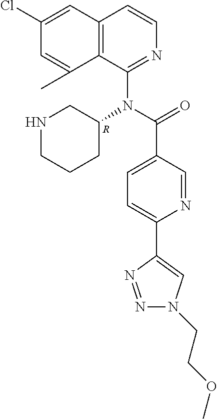 |
| 922 | (R)-4-(3,4-dihydro-2H-pyrano[3,2- c]pyridin-8-yl)-N-(8-methylisoquinolin-1- yl)-N-(piperidin-3-yl)benzamide | 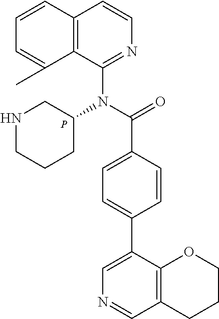 |
| 923 | (R)-2-fluoro-4-(5-methyl-4,5,6,7- tetrahydro-[1,2,3]triazolo[1,5-a]pyrazin-3- yl)-N-(3-methylthieno[3,2-c]pyridin-4-yl)- N-(piperidin-3-yl)benzamide | 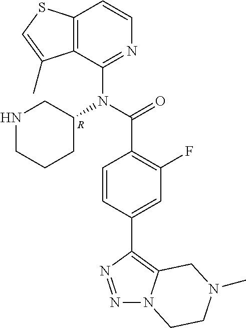 |
| 924 | (R)-N-(3-methylpyridin-2-yl)-N- (piperidin-3-yl)-4-(pyrimidin-2- ylamino)benzamide | 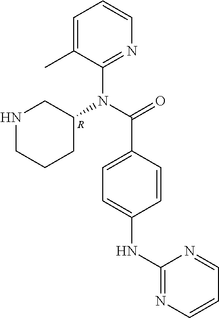 |
| 925 | (R)-N-(3-chloropyridin-2-yl)-N-(piperidin- 3-yl)-4-(pyrimidin-2-ylamino)benzamide | 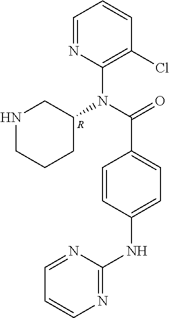 |
| 926 | (R)-2-fluoro-N-(1-methyl-1H-pyrrolo[2,3- c]pyridin-7-yl)-N-(piperidin-3-yl)-4- (pyrimidin-2-ylamino)benzamide | 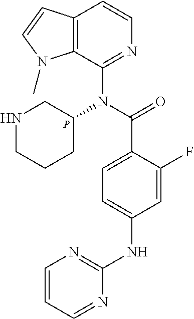 |
| 927 | (R)-2-fluoro-N-(isoquinolin-1-yl)-N- (piperidin-3-yl)-4-(pyrimidin-2- ylamino)benzamide | 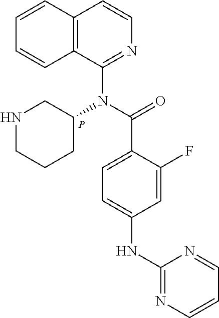 |
| 928 | (R)-2-fluoro-4-(methyl(pyrimidin-2- yl)amino)-N-(8-methylisoquinolin-1-yl)-N- (piperidin-3-yl)benzamide | 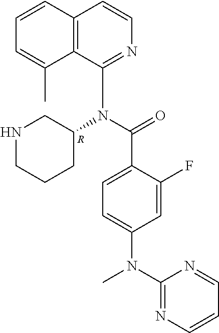 |
| 929 | methyl (R)-2-((3-fluoro-4-((8- methylisoquinolin-1-yl)(piperidin-3- yl)carbamoyl)phenyl)amino)pyrimidine-5- carboxylate | 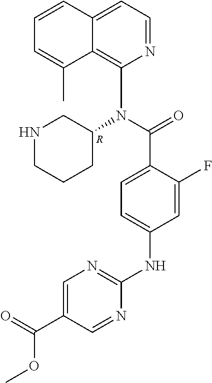 |
| 930 | (R)-2-fluoro-N-(piperidin-3-yl)-N-(3- (prop-1-en-2-yl)pyridin-2-yl)-4- (pyrimidin-2-ylamino)benzamide | 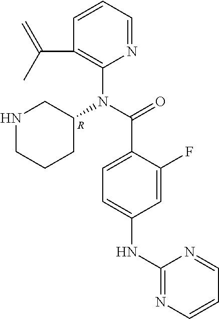 |
| 931 | (R)-2-fluoro-N-(3-methylthieno[3,2- c]pyridin-4-yl)-N-(piperidin-3-yl)-4- (pyrimidin-2-ylamino)benzamide | 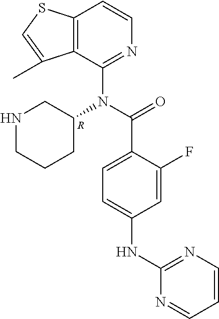 |
| 932 | (R)-4-((4-(1H-indazol-5-yl)pyrimidin-2- yl)amino)-2-fluoro-N-(8- methylisoquinolin-1-yl)-N-(piperidin-3- yl)benzamide | 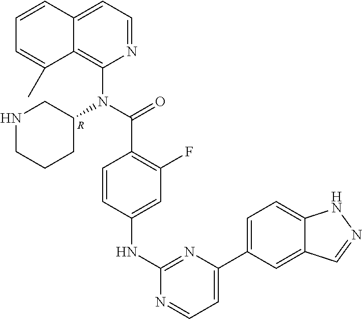 |
| 933 | (R)-4-((4-(4-acetylpiperazin-1- yl)pyrimidin-2-yl)amino)-2-fluoro-N-(8- methylisoquinolin-1-yl)-N-(piperidin-3- yl)benzamide | 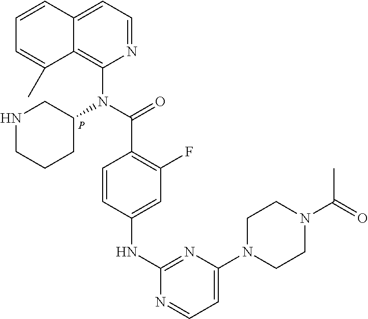 |
| 934 | (R)-2-fluoro-N-(8-methylisoquinolin-1-yl)- 4-((4-((4-methylpiperazin-1- yl)methyl)pyrimidin-2-yl)amino)-N- (piperidin-3-yl)benzamide | 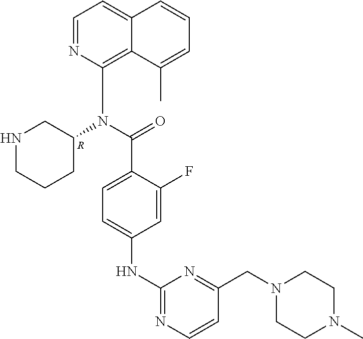 |
| 935 | (R)-4-((4-(1,1- dioxidothiomorpholino)pyrimidin-2- yl)amino)-2-fluoro-N-(8- methylisoquinolin-1-yl)-N-(piperidin-3- yl)benzamide | 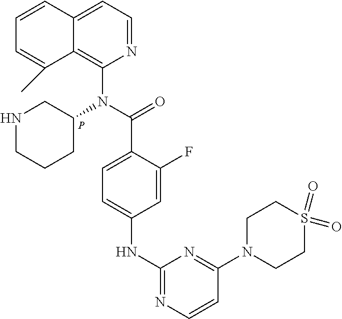 |
| 936 | (R)-2-fluoro-4-(4-(4-hydroxy-4- methylpiperidin-1-yl)pyrimidin-2- ylamino)-N-(8-methylisoquinolin-1-yl)-N- (piperidin-3-yl)benzamide | 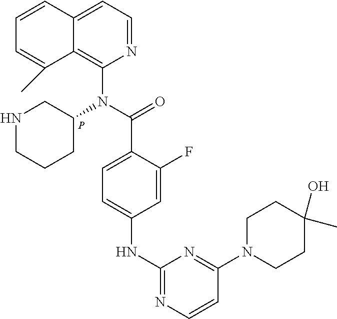 |
| 937 | (R)-2-fluoro-N-(8-methylisoquinolin-1-yl)- 4-(4-(3-oxomorpholino)pyrimidin-2- ylamino)-N-(piperidin-3-yl)benzamide | 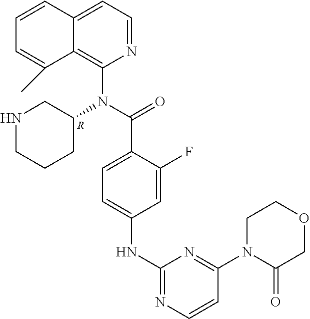 |
| 938 | (R)-2-fluoro-N-(8-methylisoquinolin-1-yl)- 4-((4-(3-oxopiperazin-1-yl)pyrimidin-2- yl)amino)-N-(piperidin-3-yl)benzamide | 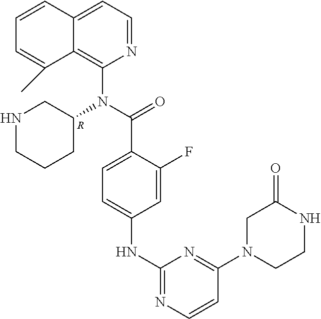 |
| 939 | 4-((4-((3R,5R)-3,5- dimethylmorpholino)pyrimidin-2- yl)amino)-2-fluoro-N-(8- methylisoquinolin-1-yl)-N-((R)-piperidin- 3-yl)benzamide | 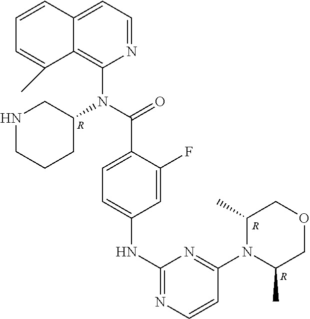 |
| 940 | 4-((4-((3R,5S)-3,5- dimethylmorpholino)pyrimidin-2- yl)amino)-2-fluoro-N-(8- methylisoquinolin-1-yl)-N-((R)-piperidin- 3-yl)benzamide | 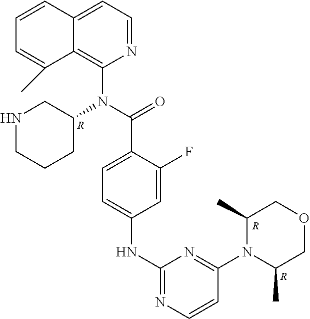 |
| 941 | 2-fluoro-4-((4-((R)-3- isopropylmorpholino)pyrimidin-2- yl)amino)-N-(8-methylisoquinolin-1-yl)-N- ((R)-piperidin-3-yl)benzamide | 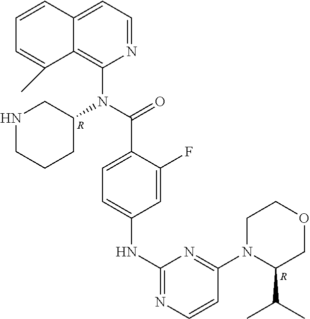 |
| 942 | 2-fluoro-4-((4-((R)-3- (hydroxymethyl)morpholino)pyrimidin-2- yl)amino)-N-(8-methylisoquinolin-1-yl)-N- ((R)-piperidin-3-yl)benzamide | 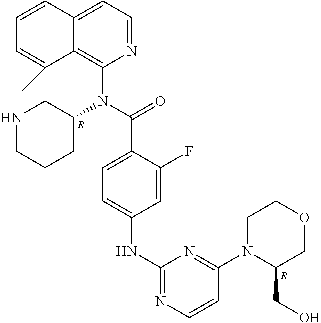 |
| 943 | 2-fluoro-4-(4-((R)-3- (methoxymethyl)morpholino)pyrimidin-2- ylamino)-N-(8-methylisoquinolin-1-yl)-N- ((R)-piperidin-3-yl)benzamide | 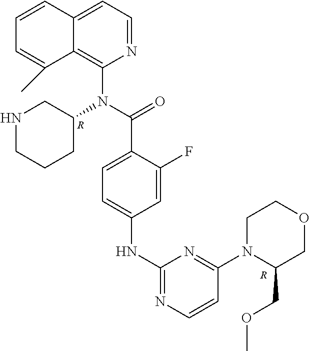 |
| 944 | (R)-4-(4-(1,4-oxazepan-4-yl)pyrimidin-2- ylamino)-2-fluoro-N-(8-methylisoquinolin- 1-yl)-N-(piperidin-3-yl)benzamide | 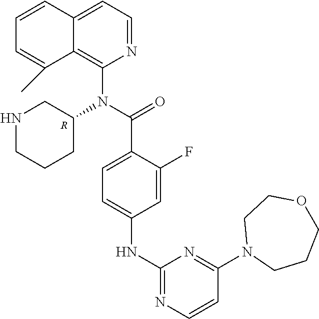 |
| 945 | methyl (S)-4-(2-((3-fluoro-4-((8- methylisoquinolin-1-yl)((R)-piperidin-3- yl)carbamoyl)phenyl)amino)pyrimidin-4- yl)morpholine-3-carboxylate | 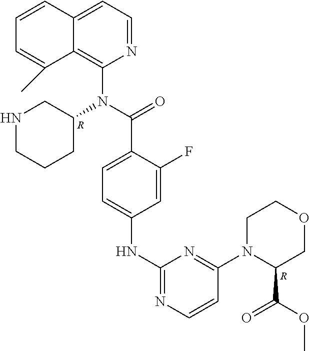 |
| 946 | 2-fluoro-4-(4-((1r,4r)-4- hydroxycyclohexylamino)pyrimidin-2- ylamino)-N-(8-methylisoquinolin-1-yl)-N- ((R)-piperidin-3-yl)benzamide | 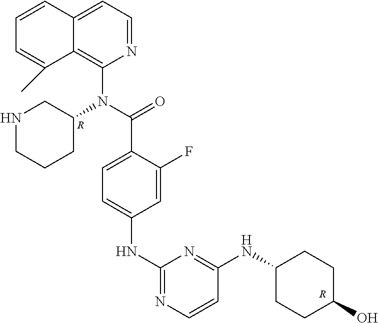 |
| 947 | (R)-2-fluoro-N-(8-methylisoquinolin-1-yl)- 4-((4-(3-oxopyrrolidin-1-yl)pyrimidin-2- yl)amino)-N-(piperidin-3-yl)benzamide | 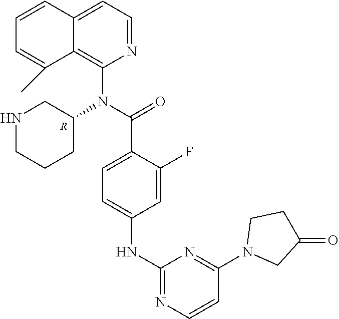 |
| 948 | 4-({4- [cyclopentyl(methyl)amino]pyrimidin-2- yl}amino)-2-fluoro-N-(8- methylisoquinolin-1-yl)-N-[(3R)-piperidin- 3-yl]benzamide | 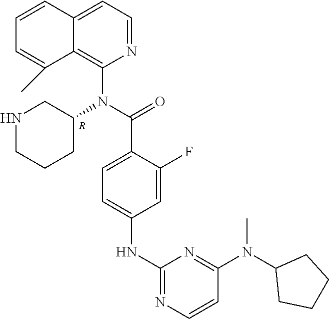 |
| 949 | (R)-4-((4-((2- cyanoethyl)(methyl)amino)pyrimidin-2- yl)amino)-2-fluoro-N-(8- methylisoquinolin-1-yl)-N-(piperidin-3- yl)benzamide | 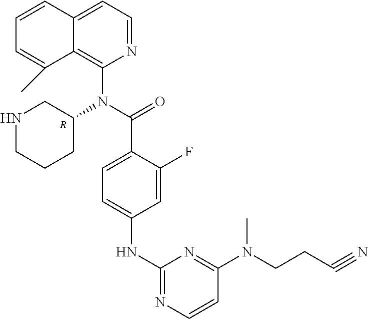 |
| 950 | (R)-4-((4-((cyanomethyl)amino)pyrimidin- 2-yl)amino)-2-fluoro-N-(8- methylisoquinolin-1-yl)-N-(piperidin-3- yl)benzamide | 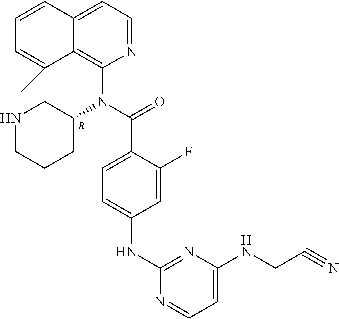 |
| 951 | 2-fluoro-4-[(4-methoxypyrimidin-2- yl)amino]-N-(8-methylisoquinolin-1-yl)-N- [(3R)-piperidin-3-yl]benzamide | 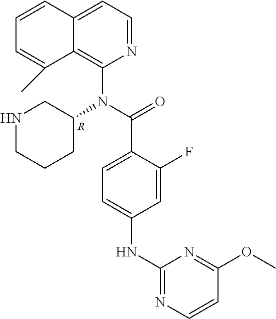 |
| 952 | 4-((4-(sec-butyl(methyl)amino)pyrimidin- 2-yl)amino)-2-fluoro-N-(8- methylisoquinolin-1-yl)-N-((R)-piperidin- 3-yl)benzamide | 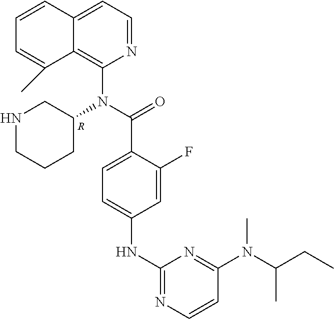 |
| 953 | (R)-4-(1,2,4-triazin-3-ylamino)-2-fluoro- N-(8-methylisoquinolin-1-yl)-N-(piperidin- 3-yl)benzamide | 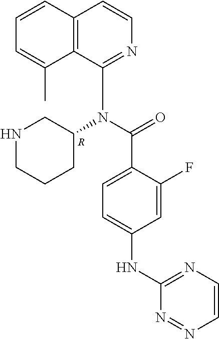 |
| 954 | (R)-2-fluoro-N-(8-methylisoquinolin-1-yl)- N-(piperidin-3-yl)-4-((4-((tetrahydro-2H- pyran-4-yl)amino)pyrimidin-2- yl)amino)benzamide | 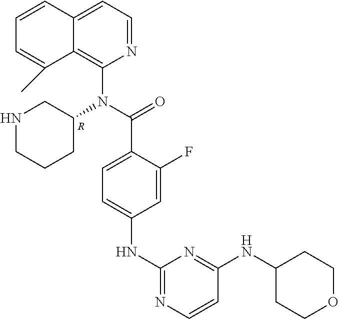 |
| 955 | 4-((4-(3-cyanomorpholino)pyrimidin-2- yl)amino)-2-fluoro-N-(8- methylisoquinolin-1-yl)-N-((R)-piperidin- 3-yl)benzamide | 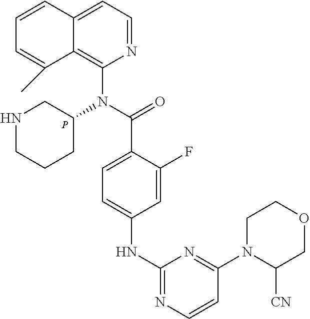 |
| 956 | 4-(4-(3- ((dimethylamino)methyl)morpholino)pyri midin-2-ylamino)-2-fluoro-N-(8- methylisoquinolin-1-yl)-N-((R)-piperidin- 3-yl)benzamide | 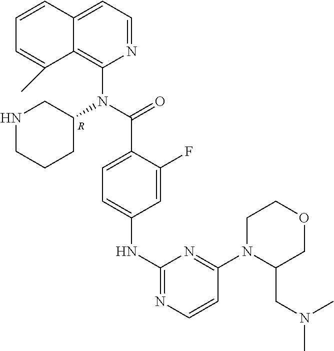 |
| 957 | (R)-2-fluoro-4-(4-(methyl(tetrahydro-2H- pyran-4-yl)amino)pyrimidin-2-ylamino)- N-(8-methylisoquinolin-1-yl)-N-(piperidin- 3-yl)benzamide | 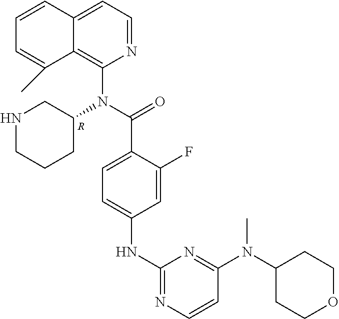 |
| 958 | 2-fluoro-N-(8-methylisoquinolin-1-yl)-N- ((R)-piperidin-3-yl)-4-((4-(3-(pyridin-3- yl)morpholino)pyrimidin-2- yl)amino)benzamide | 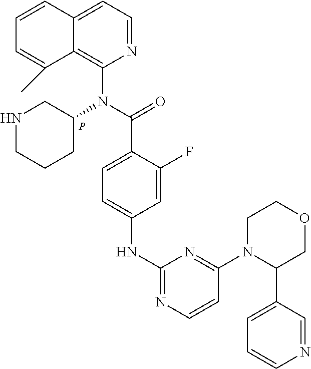 |
| 959 | 2-fluoro-N-(8-methylisoquinolin-1-yl)-4- ({4-[(2R)-2-methylpiperidin-1- yl]pyrimidin-2-yl}amino)-N-[(3R)- piperidin-3-yl]benzamide | 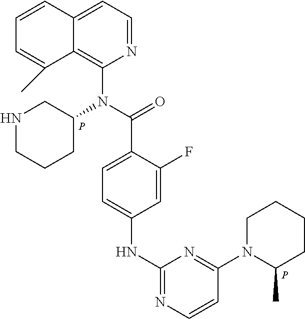 |
| 960 | 2-fluoro-4-((4-((S)-2- (hydroxymethyl)piperidin-1-yl)pyrimidin- 2-yl)amino)-N-(8-methylisoquinolin-1-yl)- N-((R)-piperidin-3-yl)benzamide | 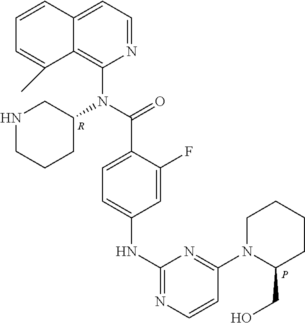 |
| 961 | (R)-2-fluoro-N-(8-methylisoquinolin-1-yl)- N-(piperidin-3-yl)-4-((4-(pyrrolidin-1- ylmethyl)pyrimidin-2-yl)amino)benzamide | 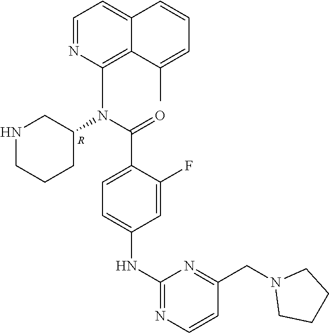 |
| 962 | (R)-2-fluoro-N-(8-methylisoquinolin-1-yl)- N-(piperidin-3-yl)-4-((4-(4-(pyridin-4- yl)piperidin-1-yl)pyrimidin-2- yl)amino)benzamide | 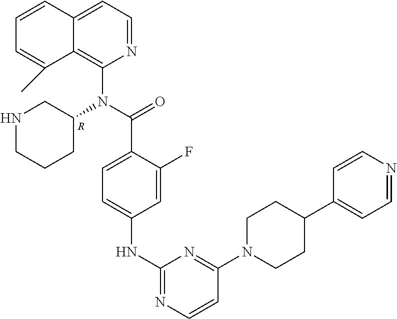 |
| 963 | 2-fluoro-4-({4-[4-(hydroxymethyl)-4- methylpiperidin-1-yl]pyrimidin-2- yl}amino)-N-(8-methylisoquinolin-1-yl)- N-[(3R)-piperidin-3-yl]benzamide | 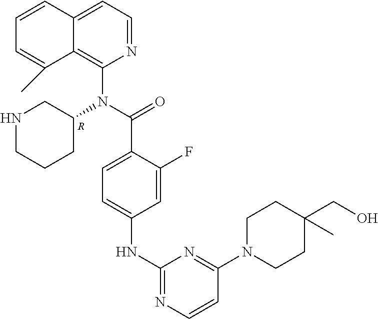 |
| 964 | (R)-2-fluoro-4-((4-(4- (methoxymethyl)piperidin-1-yl)pyrimidin- 2-yl)amino)-N-(8-methylisoquinolin-1-yl)- N-(piperidin-3-yl)benzamide | 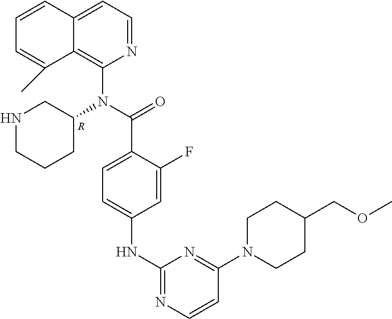 |
| 965 | methyl (2-((3-fluoro-4-((8- methylisoquinolin-1-yl)((R)-piperidin-3- yl)carbamoyl)phenyl)amino)pyrimidin-4- yl)-D-prolinae | 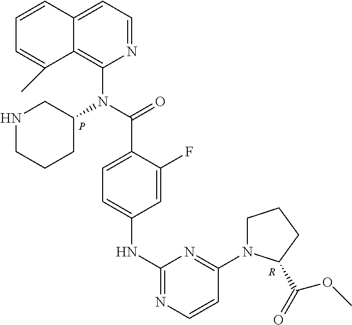 |
| 966 | 2-fluoro-4-({4-[(2R)-2- (hydroxymethyl)pyrrolidin-1-yl]pyrimidin- 2-yl}amino)-N-(8-methylisoquinolin-1-yl)- N-[(3R)-piperidin-3-yl]benzamide | 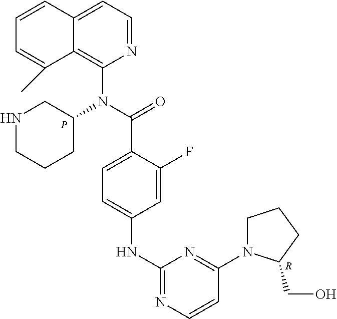 |
| 967 | 4-({4-[(3S)-3-(dimethylamino)pyrrolidin- 1-yl]pyrimidin-2-yl} amino)-2-fluoro-N-(8- methylisoquinolin-1-yl)-N-[(3R)-piperidin- 3-yl]benzamide | 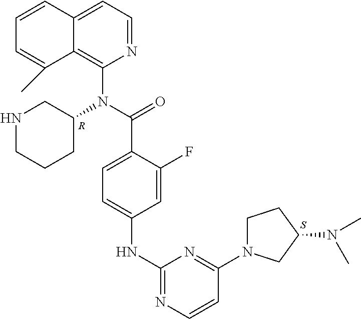 |
| 968 | 2-fluoro-N-(8-methylisoquinolin-1-yl)-N- [(3R)-piperidin-3-yl]-4-{ [4-(pyrrolidin-1- yl)-1,3,5-triazin-2-yl]amino } benzamide | 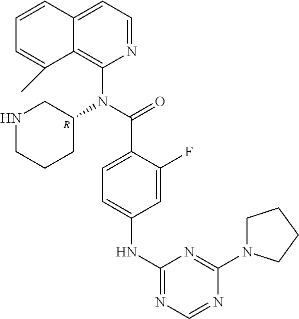 |
| 969 | 2-fluoro-N-(8-methylisoquinolin-1-yl)-4- {[4-(piperidin-1-yl)-1,3,5-triazin-2- yl]amino}-N-[(3R)-piperidin-3- yl]benzamide | 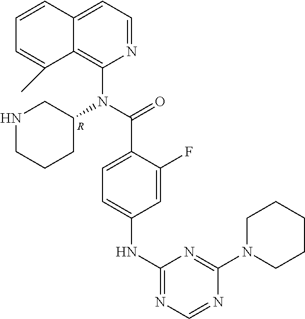 |
| 970 | 2-fluoro-N-(8-methylisoquinolin-1-yl)-4- {[4-(morpholin-4-yl)-1,3,5-triazin-2- yl]amino}-N-[(3R)-piperidin-3- yl]benzamide | 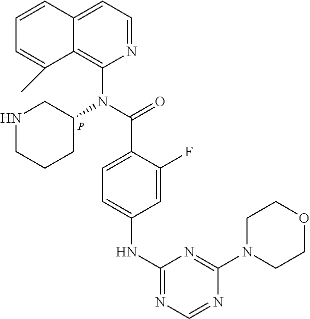 |
| 971 | (R)-2-fluoro-N-(8-methylisoquinolin-1-yl)- 4-(4-phenyl-1,3,5-triazin-2-ylamino)-N- (piperidin-3-yl)benzamide | 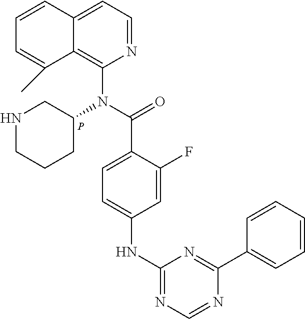 |
| 972 | (R)-4-((4-((4,4-difluoropiperidin-1- yl)methyl)pyrimidin-2-yl)amino)-N-(8- methylisoquinolin-1-yl)-N-(piperidin-3- yl)benzamide | 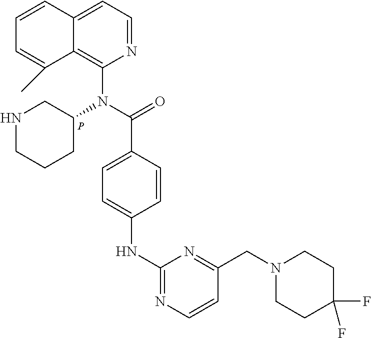 |

and pharmaceutically acceptable solvates, hydrates, salts thereof.

In some embodiments, one or more hydrogen atoms attached to carbon atoms of the compound of Formula (I) are replaced by deuterium atoms.

### General Synthetic Methods

The compounds described herein can be made using conventional organic syntheses and commercially available starting materials, or the methods provided herein. By way of example and not limitation, compounds of Formula I can be prepared as outlined in Schemes 1-12, as well as in the examples set forth herein. It should be noted that one skilled in the art would know how to modify the procedures set forth in the illustrative schemes and examples to arrive at the desired products.

Furthermore, all compounds of Formula I or any other Formula or compound as described herein which exist in free base or acid form can be converted to their pharmaceutically acceptable salts by treatment with the appropriate inorganic or organic base or acid by methods known to one skilled in the art. Salts of the compounds of the disclosure can be converted to their free base or acid form by standard techniques.

It will also be appreciated by those skilled in the art, although such protected derivatives of compounds of this disclosure may not possess pharmacological activity as such, they may be administered to a mammal and thereafter metabolized in the body to form compounds of the disclosure which are pharmacologically active. Such derivatives may therefore be described as “prodrugs.” All prodrugs of compounds of this disclosure are included within the scope of the disclosure. Further, the above general scheme can be applied to any other Formula or compound as described herein.

When enantiomerically pure or enriched compounds are desired, chiral chromatography and/or enantiomerically pure or enriched starting materials may be used as described in the Examples. It will also be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl and the like. Suitable protecting groups for amino, amidino and guanidino include t-butoxycarbonyl, benzyloxycarbonyl and the like. Suitable protecting groups for mercapto include C(O) R″ (where R″ is alkyl, aryl or arylalkyl), p methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (2006), 4th Ed., Wiley. As one of skill in the art would appreciate, the protecting group may also be a polymer resin such as a Wang resin, Rink resin or a 2-chlorotrityl-chloride resin.

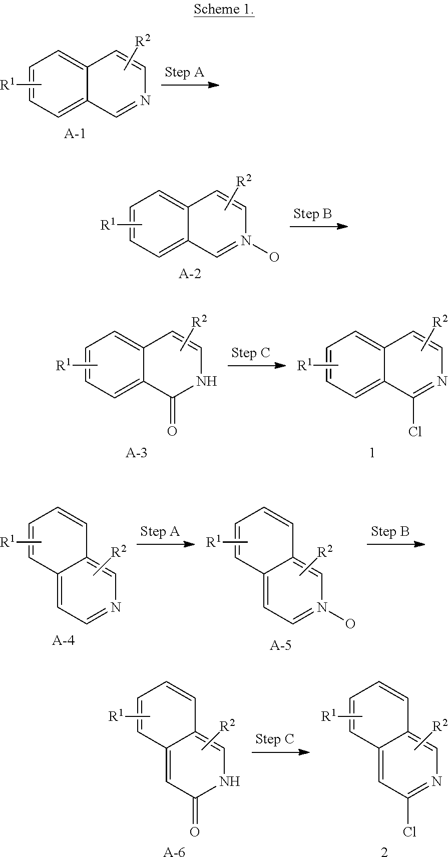

Scheme 1 above, wherein R¹ is H, halogen, alkyl, alkenyl, alkynyl, aryl, ester, carboxylic acid, amide, alkoxyl, thioether, amino, CN, heteroaryl, cycloalkyl, or heterocyclyl, and R² is H, halogen, alkyl, alkenyl, alkynyl, aryl heteroaryl, or CN provides exemplary methods for making embodiments of ring M in formula (I).

The isoquinoline building blocks 1 and 2 can be prepared according to the route illustrated in Scheme 1. Readily available isoquinolines A-1 or A-4 are converted into the corresponding N-oxide intermediate A-2 or A-5 first via treatment with an oxidant such as m-CPBA, ozone, hydrogen peroxides or KMnO₄, in a reaction medium such as THF, DCM, MeCN or acetone. Subsequently, A-2 or A-5 are converted to the corresponding isoquinolone intermediate A-3 or A-6 upon treatment of acetic anhydride with heating followed by hydrolysis with an aq. inorganic base such as NaOH, KOH or LiOH. Finally, treating A-3 or A-6 with POCl₃ and i-PrNEt₂ in an organic solvent such as MeCN, DCM or DCE with heating affords the corresponding 1-chloroisoquinoline compound 1 and compound 2.

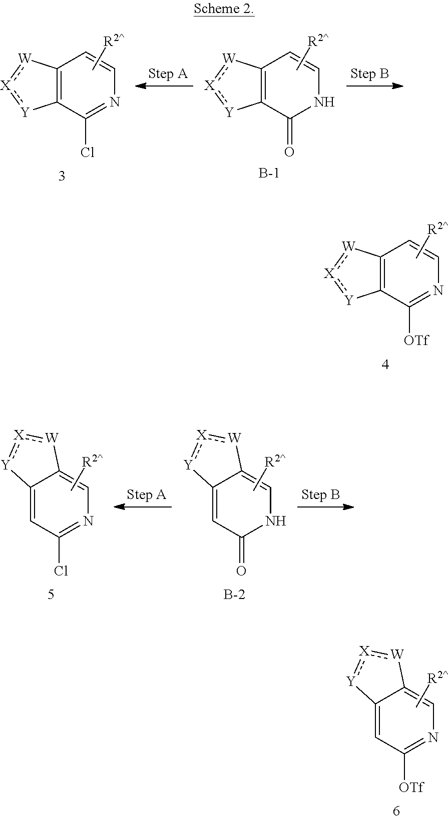

Scheme 2, above, wherein W, X, and Y are each independently CH, N, NH, N-alkyl, O, or S, and R^{2\hat{ }} is H, alkyl, alkenyl, alkynyl, cyano, or halo, provides methods of making embodiments of ring M in formula (I).

The 5,6-fused aryl building blocks 3, 4, 5, or 6 can be synthesized by utilizing two approaches as described in Scheme 2. The 5,6-fused aromatic precursors B-1 or B-2 are converted to the corresponding chloropyridine compound 3 or compound 5 upon treatment of POCl₃ or other chlorination reagents (Step A). Alternatively, the corresponding triflate building blocks compound 4 and compound 6 are prepared from precursors B-1 or B-2 by treatment of triflate anhydride in the presence of an organic base such as 4,6-lutidine, pyridine, DBU, or Et₃N, in a solvent such as DCM or THF (Step B). When W, X, or Z is NH, a suitable protecting group such as t-Boc, ester, carbamate, tetrahydropyranyl ethers (THP), sulfonyl, trimethylsilylethoxymethyl (SEM), or silyl group, are used prior to the transformation to the triflate.

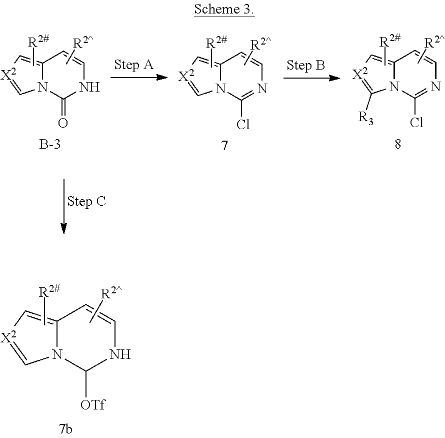

Scheme 3, above, wherein X² is CH, N, C-alkyl, C-alkenyl, C-alkynyl, C-aryl, or C-heteroaryl; R^{2\hat{ }} is described above; R^{2#} is halo, H, alkyl, alkenyl, alkynyl, cyano; and R³ is H, alkyl, alkenyl, alkynyl, halogen, cyano, ester, carboxylic acid, amide, aryl, heteroaryl, ether, thioether, or amino, provides exemplary methods for making embodiments of ring M in formula (I).

The corresponding 5,6-fused aryl building blocks 7, 7b and 8 can be prepared according to Scheme 3. The pyrimidinone precursor B-3 is converted into compound 7 upon the treatment of POCl₃ (Step A). Subsequent halogenation of compound 7 (Step B) with reagents such as NIS, NBS or NCS affords compound 8, wherein R³ is halogen. This halogen in compound 8 can be further converted into other functional groups defined by R³ above by known transformations. Alternatively, the corresponding triflate intermediate 7b can be prepared by treating B-3 with triflate anhydride as described for the preparation of compounds 4 and 6 in Scheme 2.

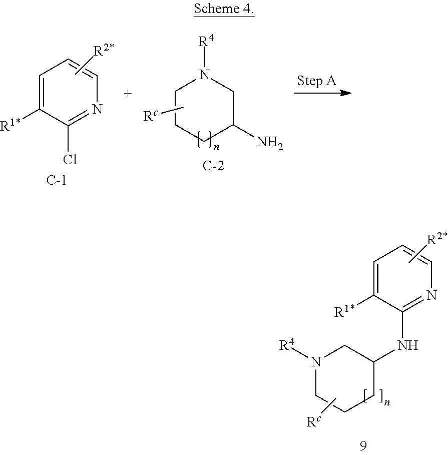

Scheme 4 above, wherein R^{1*} is H, halogen, alkyl, alkenyl, alkynyl, aryl, ester, carboxylic acid, amide, alkoxyl, thioether, cyano, heteroaryl, cycloalkyl, or heterocyclyl; R^{2*} is H, halogen, alkyl, alkenyl, alkynyl, aryl, ester, carboxylic acid, amide, alkoxyl, thioether, cyano, heteroaryl, cycloalkyl, or heterocyclyl; R^{c} is H, C₁-C₆ alkyl, OH, NH₂, fluoro, or gem-difluoro; and R⁴ is ester, t-Boc, CBz, Fmoc, or other carbamate protecting groups provides methods for making intermediate compounds useful for making compounds of Formula (I).

Compound 9 can be prepared according to the route described in Scheme 4. Precursor 2-chloropyridine C-1 is either commercially available or prepared through installations of the corresponding functional groups from commercially available precursors, such as aryl halides, by cross-coupling reactions or nucleophilic displacement reactions. Amine C-2, such as piperidine or its corresponding pyrrolidine or azepine (n=0, 1, or 2), is commercially available or is prepared from the corresponding commercially available starting materials. Coupling of C-1 and C-2 (Step A) under heating in the presence of a base such as DEA, i-PrNEt₂, K₂CO₃, or Cs₂CO₃ provides amino compound 9. Alternatively, metal-catalyzed reactions of C-1 and C-2 in the presence of a catalyst such as Pd affords amino compound 9.

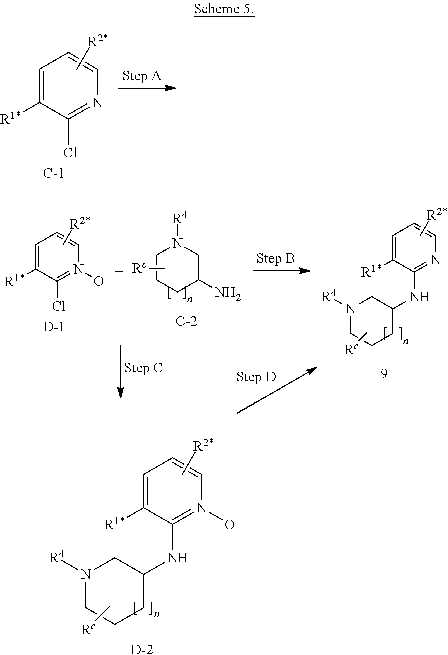

Scheme 5, above, wherein R^{1*}, R^{2*}, and R^{c} are described above and R⁴ is ester, t-Boc, CBz, Fmoc, or other carbamate protecting groups, provides methods for making intermediate compounds useful for making compounds of Formula (I).

Alternative to Scheme 4, compound 9 can be prepared in two other routes as described in Scheme 5. Precursor C-1 is converted into the N-oxide intermediate D-1 by treating with an oxidizing reagent such as m-CPBA, ozone, hydrogen peroxide, or KMnO₄, in a reaction medium such as THF, DCM, MeCN, or acetone (Step A). N-oxide D-1 is then reacted with amine building block C-2 in peptide coupling conditions, for example, with PyBop and i-PrNEt₂ in a solvent such as DMF or THF, to afford amine 9 (Step B). Alternatively, primary amine building block C-2 is converted to secondary amine D-2 via reaction at ambient temperature or with heating in a solvent such as THF, MeCN, or DMF (Step C). Subsequently, reductive conversion of the N-oxide in D-2 is achieved with a metal catalyst such as Pd or Pt with a hydrogen source or hypodiboric acid in MeCN to afford amine 9 (Step D).

Similarly, bicyclic isoquinolines in the forms of compounds 4 and 6, or the 5,6-bicyclic fused hetero building blocks in the forms of compounds 7 and 8, can be substituted for compound C-1 in Scheme 5 to provide amine products with the ring M corresponding to the starting compounds.

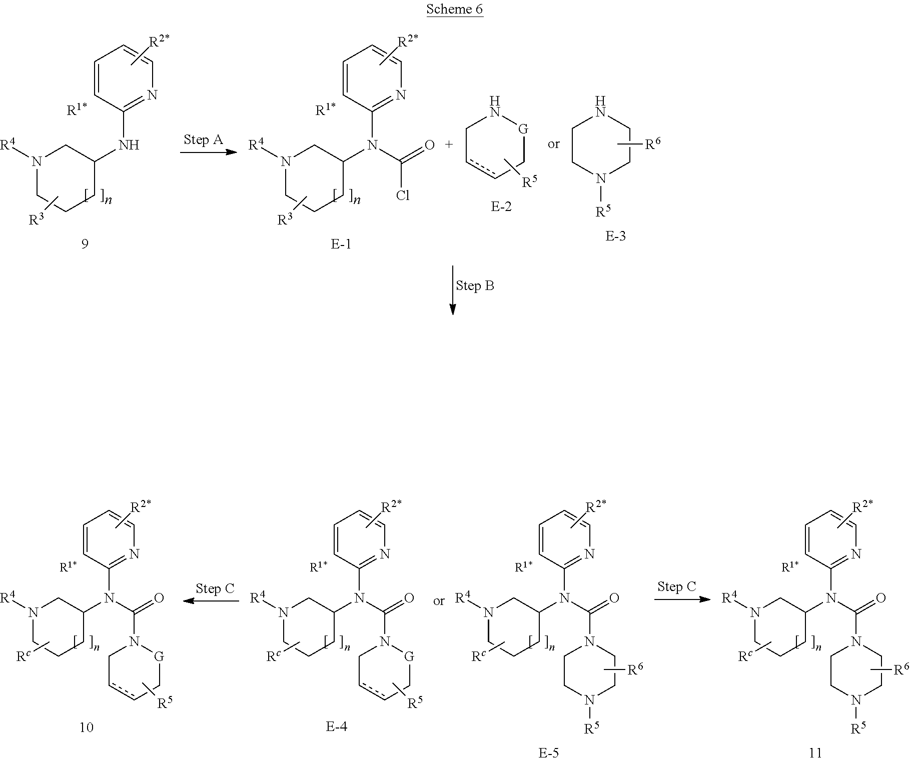

In Scheme 6 above, G is CH₂, CH-alkyl, NH, N-alkyl, or O; R^{1*}, R^{2*}, R^{c} are and R⁴ defined above; R⁵ is aryl, heteroaryl, alkyl, alkenyl, alkynyl, cycloalkyl, heterocyclyl, alkoxy, amino, cyano, carboxylic acid, ester, or amide; R⁶ is H, halogen, alkyl, alkenyl, alkynyl, aryl, ester, carboxylic acid, amide, alkoxyl, thioether, amino, cyano, heteroaryl, cycloalkyl, or heterocyclyl; and [symbol] indicates that the bond can be either a single or double bond.

Compounds 10 and 11 can be prepared according to the route described in Scheme 6. Precursor amino compound 9 is reacted with phosgene or an equivalent to provide carbamoyl chloride intermediate E-1 (Step A). Coupling of E-1 with either amine E-2 or E-3 provides the corresponding amide intermediate E-4 or E-5 (Step B). Finally, cleavage of R⁴ in E-4 and E-5 (Step C) affords compound 10 or compound 11, respectively.

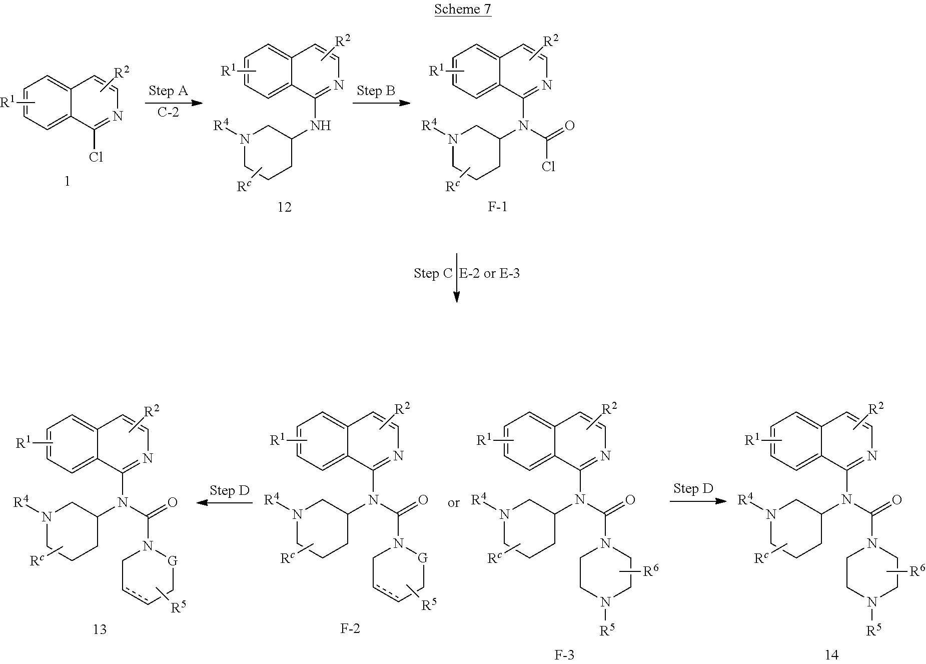

Scheme 7 above, wherein R¹, R², R^{c}, R⁴, R⁵, and R⁶ are defined above, provide methods of preparing compounds of Formula (I) and intermediates useful for preparing compounds of Formula (I).

Compounds 12-14 can be prepared from precursor 2-chloroisoquinoline 1 according to the route described in Scheme 7. Compound 12 is prepared by conjugating compound 1 and amine C-2 (Step A) via procedures similar to those as shown in the preparation of compound 9 in either Scheme 4 or Scheme 5. Conversion of compound 12 into carbamoyl chloride F-1 is performed according to the procedure described for E-1 in Scheme 6. Coupling of F-1 and the corresponding amine E-2 or E-3 in Step C provides compounds F-2 or F-3, respectively. Finally, removal of the protecting group R⁴ in F-2 or F-3 affords the products compound 13 and compound 14, respectively.

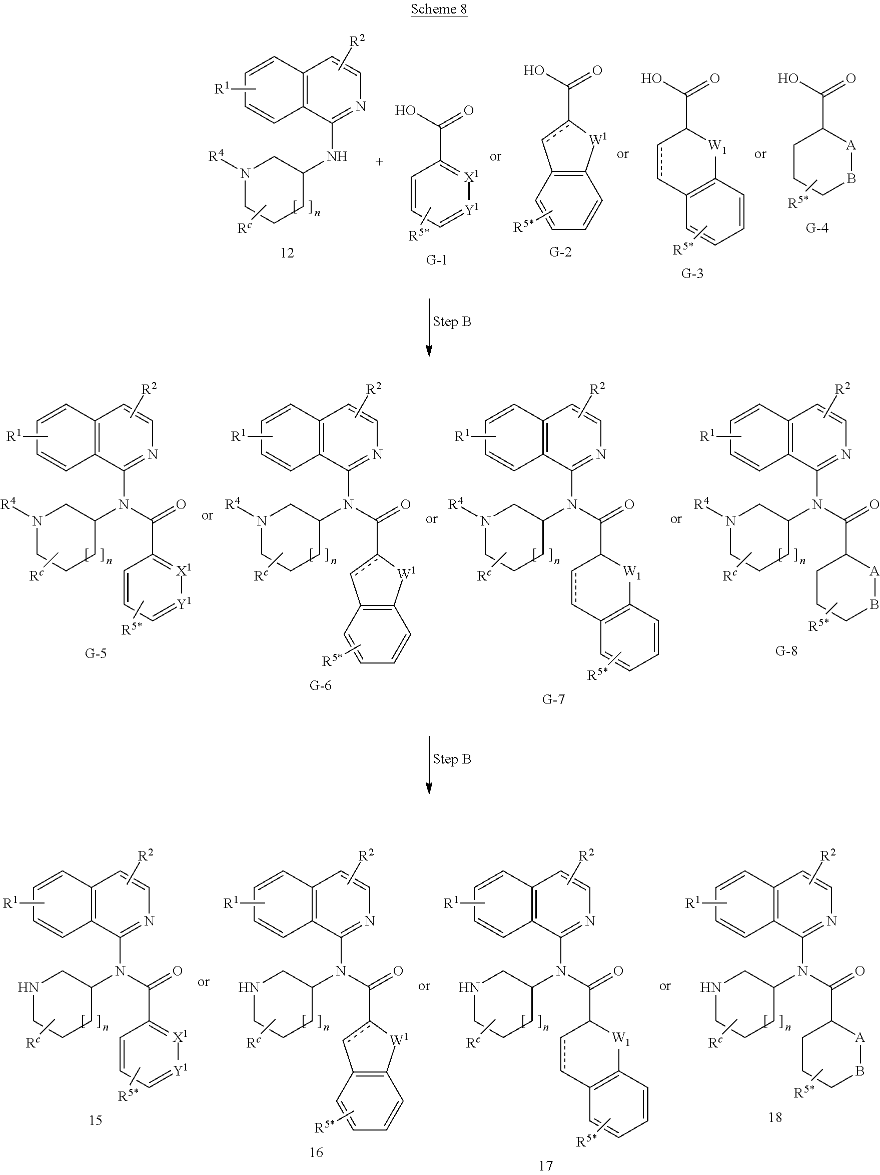

In Scheme 8, above, R¹, R², R^{c} and R⁴ are defined above; A and B are each CH₂, O, or NH; W¹ is CH₂, O, or NH; X¹ and Y¹ are each CH or N; R^{5*} is halogen, cyano, amide, alkenyl, alkynyl, aryl, heteroaryl, alkoxylaryl (aryl ether), alkoxylheteroaryl (heteroaryl ether), NH-aryl, or NH-heteroaryl; and [symbol] indicates that the bond can be either a single or double bond.

Compounds 15-19 can be prepared from precursor 2-chloroisoquinoline 12 according to the route described in Scheme 8. Coupling reaction between compound 12 and the corresponding carboxylic acid building blocks G-1, G-2, G-3, or G-4, or their activated carboxylic acids or converted acyl chlorides, under common amide formation conditions yield the four respective intermediates (G-5, G-6, G-7, or G-8) in Step A. Removal of the protecting group R⁴ in these intermediates afford the corresponding final products compounds 15, 16, 17, or 18 (Step B).

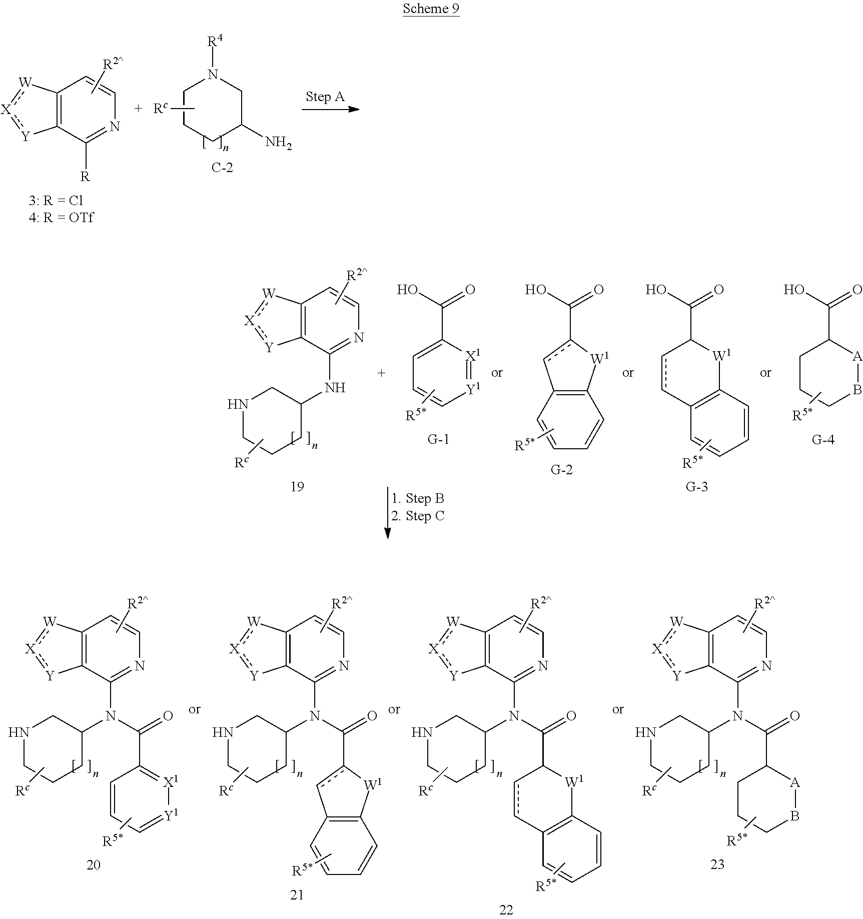

In Scheme 9 R^{2\hat{ }}, X, W, Y, R^{c}, R⁴, A, B, W¹, X¹, Y¹, and R^{5*} are defined above; and [symbol] indicates that the bond can be either a single or double bond.

Compounds 20, 21, 22, and 23 can be prepared from precursor compound 3 (R=Cl) or compound 4 (R=OTf) and the corresponding carboxylic acids (G-1, G-2, G-3, or G-4) according to the route described in Scheme 9. Reacting the precursor compound 3 with the respective carboxylic acid (G-1, G-2, G-3, or G-4) via processes similar to those described for the preparation of compounds 15, 16, 17, or 18 in Scheme 8 affords compounds 20, 21, 22, and 23.

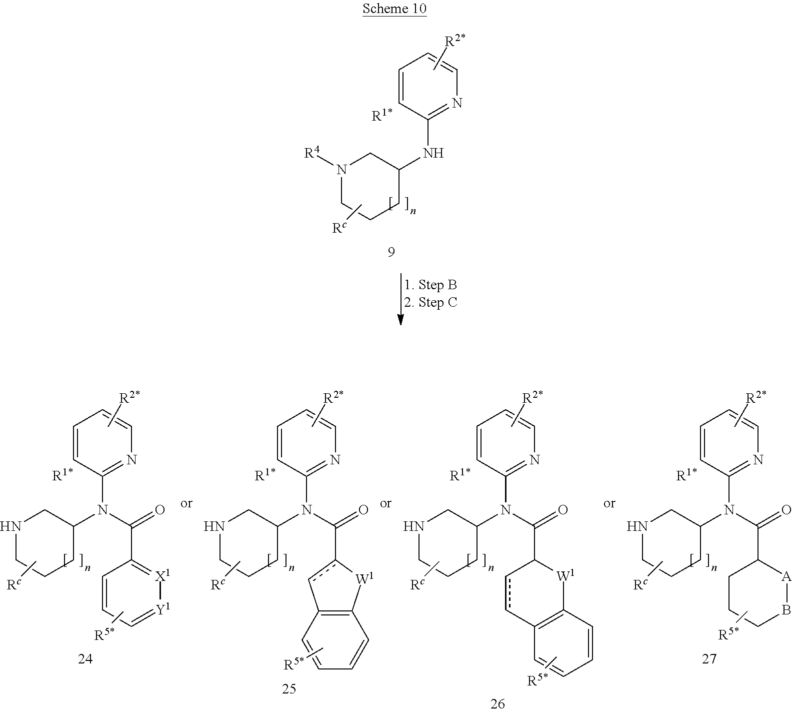

In Scheme 10, R^{1*}, R^{2*}, R^{c}, R⁴, R^{5*}, A, B, W¹, X¹ and Y¹ are as defined above.

Compounds 24, 25, 26, and 27 can be prepared from precursor 9 and the corresponding carboxylic acids (G-1, G-2, G-3, and G-4) according to the route in Scheme 10. Reacting the precursor compound 9 with the respective carboxylic acid (G-1, G-2, G-3, or G-4) via processes similar to those described for the preparation of compounds 15, 16, 17, or 18 in Scheme 8 affords compounds 24, 25, 26, and 27.

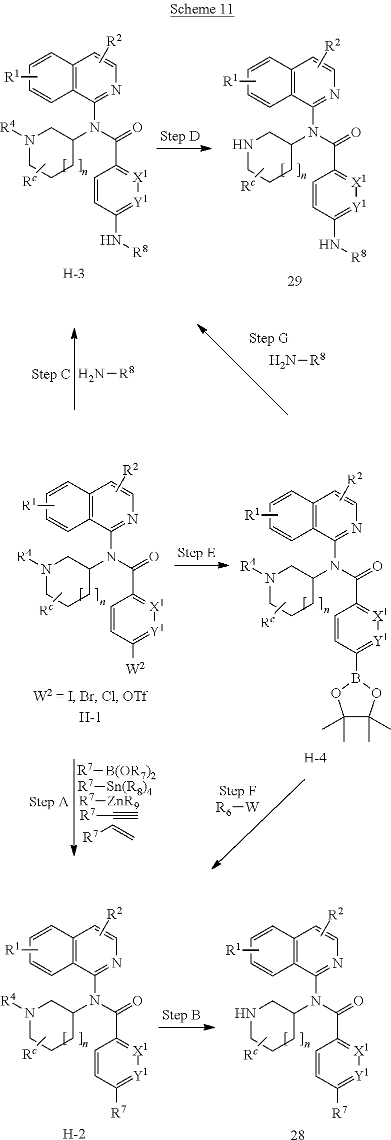

In Scheme 9, above R¹, R², R^{c}, R⁴, X¹, and Y¹ are defined above. W² is H, I, Br, Cl, or OTf; and R⁷ is alkyl, alkenyl, alkynyl, cycloalkyl, heterocyclyl, aryl, or heteroaryl; and R⁸ is H, alkyl, aryl, heteroaryl, or heterocyclyl.

Compounds 28 and 29 can be synthesized according to Scheme 11. Precursor H-1 prepared according to Scheme 8 by conversion into intermediate H-2 via a cross coupling reaction with a substrate such as boronic acid or ester reagent (Suzuki reaction), tin reagent (Stille reaction), zinc reagent (Neigishi reaction), alkynyl substrate (Sonogashira reaction), or vinyl substrate (Heck reaction) in Step A. Subsequent removal of the protecting group R⁴ in H-2 affords the final product 28 (Step B).

Alternatively, precursor H-1 is converted into the amine intermediate H-3 by treating with amine (H₂NR⁸) in the presence of a catalyst such as Pd or Cu (Step C). Removal of the R⁴ protecting group in intermediate H-3 (Step D) affords final product 29.

Alternatively, precursor H-1 is converted into the boronic ester intermediate H-4 by boronylation (Step E) upon treating with bis(pinacolacto)diboron or pinacolborane in the presence of a Pd catalyst (for W²=I, Br, Cl, or OTf) or Ir catalyst (W²═H). Subsequently, intermediate H-4 is converted to intermediate H-2 via a Suzuki coupling reaction with an aryl or alkenyl halide (Step F), followed by deprotection of the amine (Step B) to afford compound 28. To afford compound 29, Boronic ester intermediate H-4 is converted to intermediate H-3 via Chan-Lam-Evans reaction with an amine (H₂NR₈) and a Cu catalyst in the presence of an organic base such as pyridine or lutidine. Cleavage of the protecting group R⁴ of intermediate H-3 (Step D) then affords the final product 29.

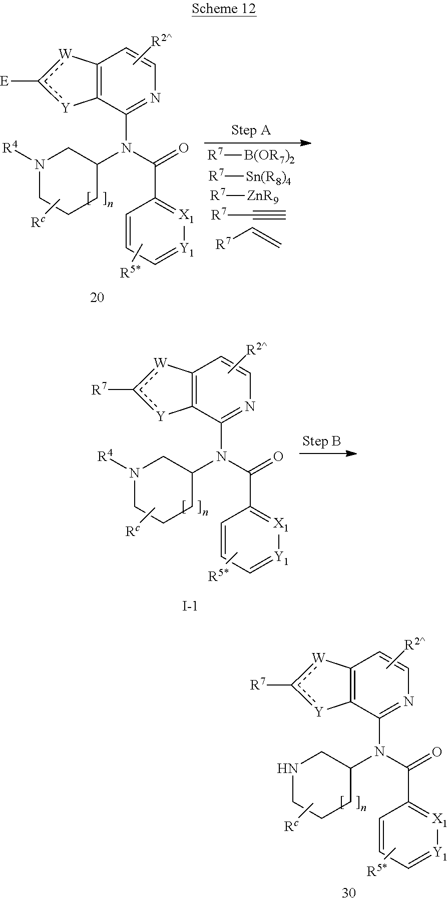

In Scheme 12, R^{2\hat{ }}, R⁴, R^{5*}, R⁷, X¹, Y¹, W, Y, and R^{c} are defined above; E is I, Br, Cl, or OTf; and the [symbol] in this scheme indicates that the bond can be either a single or double bond provided in this embodiment the five-membered ring is aromatic.

Compound 30 can be synthesized from compound 20 following procedures as described in Scheme 12. Precursor compound 20 is converted into intermediate I-1 via a cross-coupling reaction with a reagent such as boronic acid or ester reagent (Suzuki reaction), tin reagent (Stille reaction), zinc reagent (Neigishi reaction), alkynyl substrate (Sonogashira reaction), or vinyl substrate (Heck reaction) in Step A. Removal of the protecting group R⁴ in intermediate I-1 affords the final product compound 30 (Step B).

“Compounds of Formula I may be isolated as racemates, enantiomers or diastereoisomers using well-known techniques such as crystallization, chiral chromatography, or supercritical fluid chromatography. These techniques may be applied at the appropriate point in the synthesis. The Formula I compound diastereomers/enantiomers can be prepared as racemic mixtures followed by appropriate chiral separation as described herein or by for example reaction with the desired chiral starting compound. Optically active forms of the compounds can be prepared using any method known in the art, including but not limited to by resolution of the racemic form by recrystallization techniques, by synthesis from optically-active starting materials, by chiral synthesis, or by chromatographic separation using a chiral stationary phase.

Examples of methods to obtain optically active materials include at least the following.
i) physical separation of crystals: a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization: a technique whereby the individual enantiomers are separately crystallized from a solution of the racemate, possible only if the latter is a conglomerate in the solid state;
iii) cocrystallization: a technique whereby the individual enantiomers are crystallized together from a solution of the racemate;
iv) enzymatic resolutions: a technique whereby partial or complete separation of a racemate by virtue of differing rates of reaction for the enantiomers with an enzyme;
v) enzymatic asymmetric synthesis: a synthetic technique whereby at least one step of the synthesis uses an enzymatic reaction to obtain an enantiomerically pure or enriched synthetic precursor of the desired enantiomer;
vi) chemical asymmetric synthesis: a synthetic technique whereby the desired enantiomer is synthesized from an achiral precursor under conditions that produce asymmetry (i.e., chirality) in the product, which can be achieved using chiral catalysts or chiral auxiliaries;
vii) diastereomer separations: a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
viii) first- and second-order asymmetric transformations: a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
ix) kinetic resolutions: this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
x) enantiospecific synthesis from non-racemic precursors: a synthetic technique whereby the desired enantiomer is obtained from non-chiral starting materials and where the stereochemical integrity is not or is only minimally compromised over the course of the synthesis;
xi) chiral liquid chromatography: a technique whereby the enantiomers of a racemate are separated in a liquid mobile phase by virtue of their differing interactions with a stationary phase (including but not limited to via chiral HPLC). The stationary phase can be made of chiral material or the mobile phase can contain an additional chiral material to provoke the differing interactions;
xii) chiral gas chromatography: a technique whereby the racemate is volatilized and enantiomers are separated by virtue of their differing interactions in the gaseous mobile phase with a column containing a fixed non-racemic chiral adsorbent phase;
xiii) extraction with chiral solvents: a technique whereby the enantiomers are separated by virtue of preferential dissolution of one enantiomer into a particular chiral solvent;
xiv) transport across chiral membranes: a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through.

### III. COMPOSITIONS

For the purposes of administration, the compounds of the present disclosure may be administered as a raw chemical or may be formulated as pharmaceutical compositions. Pharmaceutical compositions of the present disclosure comprise a compound of Formula I and a pharmaceutically acceptable carrier, diluent or excipient. Accordingly, different embodiments are directed to pharmaceutical compositions comprising any one or more of the foregoing compounds of Formula I or a pharmaceutically acceptable salt, prodrug, stereoisomer or a mixture of stereoisomers thereof and a pharmaceutically acceptable carrier, diluent or excipient are also provided in various embodiments.

The pharmaceutical compositions of the present disclosure may be specially formulated for administration in solid or liquid form, including those adapted for the following: oral administration, for example, drenches (aqueous or non-aqueous solutions or suspensions), tablets, e.g., those targeted for buccal, sublingual and systemic absorption, boluses, powders, granules, pastes for application to the tongue; parenteral administration, for example, by subcutaneous, intramuscular, intravenous or epidural injection as, for example, a sterile solution or suspension or sustained-release formulation; topical application, for example, as a cream, ointment or a controlled-release patch or spray applied to the skin; intravaginally or intrarectally, for example, as a pessary, cream or foam; sublingually; ocularly; transdermally; or nasally, pulmonary and to other mucosal surfaces.

Provided herein are pharmaceutical compositions comprising a compound and/or a pharmaceutically acceptable salt or prodrug of the compounds of Formula (I) and a pharmaceutically acceptable excipient.

Also provided herein are compounds of Formula (I) for use as a medicament. In some embodiments, the compounds of Formula (I) are used in the manufacture of a medicament for treating a disease in a subject in which MYC contributes to the pathology and/or symptoms of the disease.

### IV. Methods

### Methods of Treating, Administration, and Pharmaceutical Compositions

Provided herein are methods of modulating c-MYC activity comprising administering an effective amount of a compound of Formula I, or a pharmaceutically acceptable salt thereof:

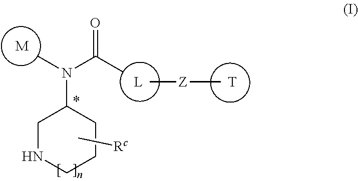

wherein
* denotes a stereocenter;
M is C₁₋₉ heteroaryl, C₁₋₉ heterocyclyl, each optionally substituted with 1, 2 or 3 substituents independently chosen from halo, substituted or unsubstituted alkyl, substituted or unsubstituted haloalkyl, substituted or unsubstituted alkenyl, substituted or unsubstituted alkynyl, substituted or unsubstituted ester, carboxylic acid, substituted or unsubstituted acetyl, substituted or unsubstituted amido, substituted or unsubstituted carbamate, substituted or unsubstituted amino, substituted or unsubstituted alkoxy, substituted or unsubstituted alkoxycarbonyl, substituted or unsubstituted thioether, cyano, substituted or unsubstituted aryl, substituted or unsubstituted heteroaryl, substituted or unsubstituted cycloalkyl, substituted or unsubstituted heterocyclyl, and combinations thereof, wherein each substituted group has 1, 2 or 3 substituents;
R^{c} is absent, C₁-C₆ alkyl, OH, NH₂, fluoro or gem-difluoro;
n is 0, 1 or 2;
L is a bond, C₆₋₁₀ arylene, C₃₋₉ heteroarylene, C₃₋₁₀ cycloalkylene, C₃₋₁₀ cycloalkenylene, or C₁₋₉ heterocyclylene, each optionally substituted with 1, 2 or 3 substituents independently chosen from halo, aryl, heteroaryl, alkyl, alkenyl, alkynyl, cycloalkyl, heterocyclyl, haloalkyl, carboxylic acid, ester, amide, alkoxy, alkoxyaryl, alkoxyheteroaryl, thioether, amino, NH-aryl, NH-heteroaryl, and cyano;
Z is a bond, —O—, S, C₁₋₃ alkylene, —(CH₂)₂C(O)NH—, —N(R^{b})—, —N(R^{b})(C₁₋₃ alkylene), where R^{b} is H, or C₁₋₃ alkyl; and
T is absent, C₆₋₁₀ aryl, C₃₋₁₀ cycloalkyl, C₂₋₉ heteroaryl, C₁₋₉ heterocyclyl, each optionally substituted with 1, 2 or 3 substituents independently chosen from OH, halo, cyano, substituted or unsubstituted aryl, substituted or unsubstituted heteroaryl, substituted or unsubstituted alkyl, haloalkyl, substituted or unsubstituted alkenyl, substituted or unsubstituted alkynyl, substituted or unsubstituted cycloalkyl, substituted or unsubstituted heterocyclyl, substituted or unsubstituted alkoxycarbonyl, hydroxycarbonyl, substituted or unsubstituted ester, substituted or unsubstituted amido, substituted or unsubstituted alkoxy, substituted or unsubstituted alkoxyaryl, substituted or unsubstituted alkoxyheteroaryl, substituted or unsubstituted thioether, substituted or unsubstituted amino, NH-aryl, and NH-heteroaryl, wherein each substituted group has 1, 2 or 3 substituents;
provided that when L is a bond, T is not absent and when T is absent, then L is not a bond.

In some embodiments, the method of modulating c-MYC activity comprises administering an effective amount of any of the compounds described herein.

In some embodiments, the MYC activity is modulated in a cell. In some embodiments, the MYC activity is modulated in a cellular assay. In some embodiments, MYC activity is modulated in an In Vitro Multiple Point Translation Screening Assay.

In some embodiments, the MYC activity is modulated in a subject in need thereof, wherein the subject is a model animal or a human. In some embodiments, the MYC activity modulates transcriptional regulation of a gene that is a biomarker. In some embodiments, the MYC activity modulates transcriptional regulation of a gene that is a biomarker that predicts anti-MYC activity.

In some embodiments, the compound of Formula I inhibits MYC activity. In some embodiments, the compound of Formula I activates MYC activity.

Also provided herein are methods of treating a condition associated with MYC activity in a subject in need thereof comprising administering to the subject an effective amount of a compound of Formula I, as defined herein or a pharmaceutically acceptable salt thereof.

In some embodiments, the method of treating a condition associated with MYC activity in a subject in need thereof comprises administering an effective amount of any of the compounds described herein.

In some embodiments, the condition associated with MYC activity is cancer. In some embodiments, the cancer is chosen from solid and hematopoietic cancers such as breast, prostate, pancreatic, hepatocellular, renal, multiple myeloma, lymphoma, lung, skin, oral squamous, haemopoietic, and neuroblastoma. In some embodiments, the cancer is derived from breast, colon, lung or hematopoietic lineages.

In some embodiments, the method further comprises administering to the subject an effective amount of a second anti-cancer agent. In some embodiments, the second anti-cancer agent is chosen from PARP inhibitors, immune oncology (I/O) drugs, tyrosine kinase inhibitors, CHK1 inhibitor, GLS inhibitor, SAE1/2 modulators, BUD31 modulators, TRRAP modulators and BRD4 modulators. In some embodiments, the second anti-cancer agent is chosen from Olaparib (Lynparza), rucaparib (Rubraca), niraparib (Zejula), nivolumab (Opdivo), atezolizumab (Tecentriq), pembrolizumab (Keytruda), avelumab (Bavencio), durvalumab (Imfinzi) and cemiplimab-rwlc (Libtayo), axitinib (Inlyta) dasatinib (Sprycel), erlotinib (Tarceva), imatinib (Glivec), nilotinib (Tasigna), pazopanib (Votrient), sunitinib (Sutent).

In some embodiments, the method comprises treating cancer by administering to a patient in need thereof a therapeutically effective amount of a compound that selectively inhibits the translation of c-MYC mRNA to c-MYC protein. In some embodiments, translation stalls by targeting a cytosolic nascent chain/ribosome complex. In some embodiments, a ternary complex between small molecule, ribosome and nascent c-MYC polypeptide is visible by Cryo-electron microscopy. In some embodiments, translation is stalled to deliver 50% partial inhibition of translation. In some embodiments, translation is stalled resulting in 90% partial inhibition of translation.

In some embodiments, the compound of Formula (I) has an IC50 below about 50 μM in an In Vitro Multiple Point Translation Screening Assay In some embodiments, the compound has an IC50 below about 5 μM in an In Vitro Multiple Point Translation Screening Assay. In some embodiments, the selective inhibition of MYC is such that less than 1% of non-MYC proteins are inhibited in a Global Proteomic Assay. In some embodiments, the selective inhibition of MYC is such that less than 0.1% of non-MYC proteins are inhibited in a Global Proteomic Assay.

Also provided herein are methods of treating cancer in a subject in need thereof comprising administering to the subject an effective amount of a compound of Formula I, as defined herein, or a pharmaceutically acceptable salt thereof. In some embodiments, the cancer is chosen from solid and hematopoietic cancers such as breast, prostate, pancreatic, hepatocellular, renal, multiple myeloma, lymphoma, lung, skin, oral squamous, haemopoietic, and neuroblastoma. In some embodiments, the cancer is derived from breast, colon, lung or hematopoietic lineages.

In general, the compounds of this disclosure will be administered in a therapeutically effective amount by any of the accepted modes of administration for agents that serve similar utilities. Therapeutically effective amounts of compounds of Formula (I) may range from about 0.01 to about 500 mg per kg patient body weight per day, which can be administered in single or multiple doses. In one embodiment, the dosage level will be about 0.1 to about 250 mg/kg per day. In another embodiment the dosage level will be about 0.5 to about 100 mg/kg per day. A suitable dosage level may be about 0.01 to about 250 mg/kg per day, about 0.05 to about 100 mg/kg per day, or about 0.1 to about 50 mg/kg per day. Within this range the dosage can be about 0.05 to about 0.5, about 0.5 to about 5 or about 5 to about 50 mg/kg per day. For oral administration, the compositions may be provided in the form of tablets containing about 1.0 to about 1000 milligrams of the active ingredient, particularly about 1.0, 5.0, 10, 15, 20, 25, 50, 75, 100, 150, 200, 250, 300, 400, 500, 600, 750, 800, 900, and 1000 milligrams of the active ingredient. The actual amount of the compound of this disclosure, i.e., the active ingredient, will depend upon numerous factors such as the severity of the disease to be treated, the age and relative health of the subject, the potency of the compound being utilized, the route and form of administration, and other factors.

In general, compounds of this disclosure will be administered as pharmaceutical compositions by any one of the following routes: oral, systemic (e.g., transdermal, intranasal or by suppository), or parenteral (e.g., intramuscular, intravenous, or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

Pharmaceutical compositions can be formulated using one or more pharmaceutically acceptable carriers comprising excipients and auxiliaries. The formulation can be modified depending upon the route of administration chosen. The pharmaceutical compositions can also include the compounds described herein in a free base form or a pharmaceutically acceptable salt or prodrug form.

Methods for formulation of the pharmaceutical compositions can include formulating any of the compounds described herein with one or more inert, pharmaceutically acceptable excipients or carriers to form a solid, semi-solid, or liquid composition. Solid compositions can include, for example, powders, tablets, dispersible granules and capsules, and in some aspects, the solid compositions further contain nontoxic, auxiliary substances, for example wetting or emulsifying agents, pH buffering agents, and other pharmaceutically acceptable additives. Alternatively, the compositions described herein can be lyophilized or in powder form for re-constitution with a suitable vehicle, e.g., sterile pyrogen-free water, before use. The active ingredients can be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

The pharmaceutical compositions and formulations can be sterilized. Sterilization can be accomplished by filtration through sterile filtration.

The pharmaceutical compositions described herein can be formulated for administration as an injection. Non-limiting examples of formulations for injection can include a sterile suspension, solution, or emulsion in oily or aqueous vehicles. Suitable oily vehicles can include, but are not limited to, lipophilic solvents or vehicles such as fatty oils, synthetic fatty acid esters, or liposomes. Aqueous injection suspensions can contain substances which increase the viscosity of the suspension. The suspension can also contain suitable stabilizers. Injections can be formulated for bolus injection or continuous infusion.

For parenteral administration, the compounds can be formulated in a unit dosage injectable form (e.g., solution, suspension, emulsion) in association with a pharmaceutically acceptable parenteral vehicle. Such vehicles can be inherently nontoxic, and non-therapeutic. A vehicle can be water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability (e.g., buffers and preservatives).

Sustained-release preparations can also be prepared. Examples of sustained-release matrices can include polyesters, hydrogels (e.g., poly(2-hydroxyethyl-methacrylate), or poly(vinylalcohol)), polylactides, copolymers of L-glutamic acid and γ ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(−)-3-hydroxybutyric acid.

Pharmaceutical formulations of the compositions described herein can be prepared for storage by mixing a compound with a pharmaceutically acceptable carrier, excipient, and/or a stabilizer. This formulation can be a lyophilized formulation or an aqueous solution. Acceptable carriers, excipients, and/or stabilizers can be nontoxic to recipients at the dosages and concentrations used. Acceptable carriers, excipients, and/or stabilizers can include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

The compounds, pharmaceutical compositions, and methods of the present disclosure can be useful for treating a subject such as, but not limited to, a mammal, a human, a non-human mammal, a domesticated animal (e.g., laboratory animals, household pets, or livestock), a non-domesticated animal (e.g., wildlife), a dog, a cat, a rodent, a mouse, a hamster, a cow, a bird, a chicken, a fish, a pig, a horse, a goat, a sheep, or a rabbit. In preferred embodiments, compounds, pharmaceutical compositions, and methods of the present disclosure are used for treating a human.

The compounds, pharmaceutical compositions, and methods described herein can be useful as a therapeutic, for example a treatment that can be administered to a subject in need thereof. A therapeutic effect can be obtained in a subject by reduction, suppression, remission, or eradication of a disease state, including, but not limited to, a symptom thereof. A therapeutic effect in a subject having a disease or condition, or pre-disposed to have or is beginning to have the disease or condition, can be obtained by a reduction, a suppression, a prevention, a remission, or an eradication of the condition or disease, or pre-condition or pre-disease state.

In practicing the methods described herein, therapeutically effective amounts of the compounds or pharmaceutical compositions described herein can be administered to a subject in need thereof, often for treating and/or preventing a condition or progression thereof. A pharmaceutical composition can affect the physiology of the subject, such as the immune system, inflammatory response, or other physiologic affect. A therapeutically effective amount can vary widely depending on the severity of the disease, the age and relative health of the subject, the potency of the compounds used, and other factors.

Treat and/or treating can refer to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treat can be used herein to refer to a method that results in some level of treatment or amelioration of the disease or condition and can contemplate a range of results directed to that end, including but not restricted to prevention of the condition entirely.

Prevent, preventing, and the like can refer to the prevention of the disease or condition in the patient. For example, if an individual at risk of contracting a disease is treated with the methods of the present disclosure and does not later contract the disease, then the disease has been prevented, at least over a period of time, in that individual.

A therapeutically effective amount can be the amount of a compound or pharmaceutical composition or an active component thereof sufficient to provide a beneficial effect or to otherwise reduce a detrimental non-beneficial event to the individual to whom the composition is administered. A therapeutically effective dose can be a dose that produces one or more desired or desirable (e.g., beneficial) effects for which it is administered, such administration occurring one or more times over a given period of time. An exact dose can depend on the purpose of the treatment and can be ascertainable by one skilled in the art using known techniques.

The compounds or pharmaceutical compositions described herein that can be used in therapy can be formulated and dosages established in a fashion consistent with good medical practice taking into account the disorder to be treated, the condition of the individual patient, the site of delivery of the compound or pharmaceutical composition, the method of administration and other factors known to practitioners. The compounds or pharmaceutical compositions can be prepared according to the description of preparation described herein.

One of ordinary skill in the art would understand that the amount, duration, and frequency of administration of a pharmaceutical composition or compound described herein to a subject in need thereof depends on several factors including, for example but not limited to, the health of the subject, the specific disease or condition of the patient, the grade or level of a specific disease or condition of the patient, the additional therapeutics the subject is being or has been administered, and the like.

The methods, compounds, and pharmaceutical compositions described herein can be for administration to a subject in need thereof. Often, administration of the compounds or pharmaceutical compositions can include routes of administration, non-limiting examples of administration routes include intravenous, intraarterial, subcutaneous, subdural, intramuscular, intracranial, intrasternal, intratumoral, or intraperitoneally. Additionally, a pharmaceutical composition or compound can be administered to a subject by additional routes of administration, for example, by inhalation, oral, dermal, intranasal, or intrathecal administration.

Pharmaceutical compositions or compounds of the present disclosure can be administered to a subject in need thereof in a first administration, and in one or more additional administrations. The one or more additional administrations can be administered to the subject in need thereof minutes, hours, days, weeks, or months following the first administration. Any one of the additional administrations can be administered to the subject in need thereof less than 21 days, or less than 14 days, less than 10 days, less than 7 days, less than 4 days or less than 1 day after the first administration. The one or more administrations can occur more than once per day, more than once per week, or more than once per month. The compounds or pharmaceutical compositions can be administered to the subject in need thereof in cycles of 21 days, 14 days, 10 days, 7 days, 4 days, or daily over a period of one to seven days.

The MYC gene family is a family of proto-oncogenes that encode three nuclear phosphoproteins that play critical roles in cell cycle progression, apoptosis and cellular transformation, as defined and referenced above. The encoded c-MYC protein forms a heterodimer with the related transcription factor MAX. This complex binds to the E box DNA consensus sequence and regulates the transcription of specific target genes. Amplification of this gene is frequently observed in numerous human cancers. Translocations involving this gene are associated with Burkitt lymphoma and multiple myeloma in human patients. There is evidence to show that translation initiates both from an upstream, in-frame non-AUG (CUG) and a downstream AUG start site, resulting in the production of two isoforms with distinct N-termini. Another target of interest in the MYC family is N-MYC as defined and referenced above. Despite numerous attempts (reviewed in Liombart 2022), it has proven technically challenging to discover potent drug-like inhibitors of c-MYC or n-MYC and no c-MYC or n-MYC inhibitors have advanced into phase 2 human clinical testing or beyond.

The present disclosure is based in part on the discovery of a new approach to targeting c-MYC or n-MYC that directly circumvents these issues above. In this approach translation stallers target a cytosolic nascent chain/ribosome complex instead of a fully translated protein. Translation can be tuned to deliver partial inhibition and thus impact the therapeutic index. Accordingly, one aspect described herein provides a method of treating cancers, the method comprising administering to a subject in need thereof an effective amount of an agent that modulates c-MYC or n-MYC.

Another aspect described herein provides a method of inhibiting c-MYC or n-MYC, the method comprising administering to a subject in need thereof an effective amount of an agent that modulates c-MYC or n-MYC.

Dysregulation of c-MYC in human cancer. Literature evidence supports the hypothesis that inhibitors of c-MYC translation or n-MYC translation will have utility for treatment of various cancers

In another aspect, described herein are methods of treating cancer, the method comprising administering to a subject in need thereof an effective amount of an agent that modulates MYC.

In some embodiments the cancer is chosen from breast, prostate, pancreatic, hepatocellular, renal, multiple myeloma, lymphoma, lung, skin, oral squamous, haemopoietic, and neuroblastoma.

The oncogenes c-MYC and n-MYC are dysregulated in a variety of cancers and are known to promote cancer growth. The MYC family of transcription factors are master regulators of gene expression. Notably, c-MYC is believed to modulate the expression of up to 15% of all expressed human genes. Genetic aberrations such as chromosomal translocations, gene amplification or aberrant transcriptional regulation of the c-MYC gene or stabilization of the c-MYC protein are among the most common causes of cancer in human (Reviewed in Dang 2012; Dhanashekaran 2022) and implicated in up to 70% of all cancers. In a number of cancer types, c-MYC is frequently amplified in the extrachromosomal DNA during tumor evolution (Turner 2017). Pharmacological or genetic inhibition of c-MYC or n-MYC in cellular and animal models have shown that this is a viable strategy for treatment of cancer and is considered to be an attractive target for cancer drug discovery (Madden 2021, Dhanashekaran 2020, Chen 2018).

c-MYC Over-Expression Implicated in Dysregulation of Many Cancer Cell Lines

There is extensive evidence to support the hypothesis that overexpression of c-MYC and/or n-MYC play causative roles in dysregulated growth of diverse human cancers. Exemplary literature reports are given in Table 2 and the supporting explanatory text below.

TABLE 2
Human cancer lines for which there is literature evidence that
overexpression of c-MYC and/or n-MYC play a causative role.

| Cancer type | Cell lines |
|---|---|
| Breast | MCF-7, MDA-MB-231, CAL51, SUM149, |
| | SUM159 |
| Prostate | PC-3, 22Rv1, DU-145, LNCaP, MYC-CaP |
| Pancreatic | PANC-1, SW1990 |
| Hepatocellular | HepG2 |
| Renal | RXF393, CAKI-1 |
| Multiple myeloma | JJN-3, OPM-2, OPM-1, HP29, KMS-11, |
| | MM.1S, RPMI-8226, KMS-20, L-363, Dox40, |
| | AMO-1 |
| Lymphoma | SUP-M2, K562 |
| Lung | PC-9, A549, H23, H292 |
| Skin | A431, melanomas A375, MUM-2B, 32 and WM |
| | 266-4 |
| Oral squamous | ORL-136T |
| Haemopoietic | K562, NB4, Nalm6, HL60, SUP-M2 |
| Neuroblastoma | SK-N-BE2, IMR32 |

### Breast Cancer.

Knockdown of c-MYC expression by RNAi inhibits MCF-7 tumor cells in vitro and in vivo (Wang Y 2004). Inhibition of c-MYC expression with the CDK inhibitor Dinaciclib results in apoptosis and tumor volume particularly in MDA-MB-231 cells (Horiuchi 2015). Combination of c-MYC inhibitors with the PARP inhibitor Niraparib is synergistic (Carey 2018). Knockdown of c-MYC using siRNA directed to c-MYC results in a drop in cell viability particularly in MDA-MB-231 and SUM149 (Carey 2018).

Small molecule GSPT1/2 degraders inhibit c-MYC driven cancers in vivo (Monte Rosa WO2021/069705 A1) with MDA-MB-231 cells and CAL51 cells.

### Prostate Cancer.

Honokiol, a natural product, shows significant anti-cancer activity against prostate cancer cell lines PC-3 and 22 Rv1. It results in a marked decrease in levels of total and/or phosphorylated c-MYC protein as well as its mRNA expression (Hahm 2016). Han et al (2020) discovered a series of small molecule c-MYC inhibitors that engage c-MYC inside cells, disrupt c-MYC/MAX dimers, and impair c-MYC-driven gene expression. The compounds enhance c-MYC phosphorylation on threonine-58, consequently increasing proteasome-mediated c-MYC degradation. The initial lead, c-MYC inhibitor 361 (MYCi361), suppressed in vivo tumor growth in mice.

### Pancreatic.

Silencing of c-MYC using si-RNA inhibits proliferation of pancreatic cancer and enhances the activity of the cardiotonic steroid Bufalin (Liu 2020).

### Hepatocellular Carcinoma.

Anti-sense oligonucleotides (ASO) directed to c-MYC suppress c-MYC-driven tumorigenesis in vivo in autochthonous transgenic mouse models of c-MYC-driven HCC and RCC, and in a Hep-G2 xenograft model of human liver cancer (Danasekharan 2020).

### Renal Cell Carcinoma.

Stauprimide is a staurosporine analogue that promotes embryonic stem cell (ESC) differentiation by inhibiting nuclear localization of the c-MYC transcription factor NME2, which in turn results in downregulation of c-MYC transcription and inhibition of renal cancer cells RXF393, CAKI-1 in vitro and in vivo (Bouvard 2017).

### Multiple Myeloma.

Using precise genome-engineering approaches, Steinberger (2019) et al developed a phenotypic screen capable of identifying c-MYC expression modulators. They found that a number of Na/K ATPase inhibitors such as Bufalin, Ouabain etc inhibit c-MYC expression and inhibit cell viability of multiple myeloma cells such as JJN-3, OPM-2, OPM-1, HP29, KMS-11, MM.1S.

Using a selective small molecule bromodomain inhibitor, JQ1, Delmore et al (2011) identify BET bromodomain proteins as regulatory factors for c-MYC. BET inhibition by JQ1 downregulates MYC transcription, followed by genome-wide downregulation of c-MYC-dependent target genes. JQ1 is efficacious in three murine models of MM. Establishing the therapeutic rationale for inhibiting pathologic activation of c-MYC. Felsenstein report a novel G4-binding small molecule that inhibits c-MYC expression in cell models. The compound induced G1 arrest and was selectively toxic to c-MYC driven cancer cell lines containing the G4 in the promoter but had minimal effects in peripheral blood mononucleocytes or a cell line lacking the G4 in its c-MYC promoter (Felsenstein, 2016).

### Lymphoma.

AZD9150, an antisense oligonucleotide (ASO), decreased STAT3 expression in a broad range of preclinical cancer models and showed antitumor activity in lymphoma (SUP-M2) and lung cancer models (Hong 2015). AZD9150 preclinical activity translated into single-agent antitumor activity in patients with highly treatment-refractory lymphoma and non-small cell lung cancer in a phase I dose escalation study. In this same study another derivative showed a concentration-dependent decrease in both c-MYC mRNA and protein levels in K562 cells (human erythroleukemia cells).

### Lung Cancer.

Inhibition of STAT3 a known activator of c-MYC by antisense oligonucleotide AZD9150 (Hong 2015) or STX-0119 (Kang 2019) inhibit survival of lung cancer cells in vitro and in vivo.

Sriratanaska (2020) show that a novel c-MYC-targeting compound, N, N-bis(5-ethyl-2-hydroxybenzyl) methylamine (EMD) exhibited strong cytotoxicity toward various human lung cancer cell lines (H23 and H292), as well as chemotherapeutic-resistant patient-derived lung cancer cells, through apoptosis induction. EMD strongly induced the ubiquitination of c-MYC and promoted protein degradation.

### Skin Cancer.

Inhibition of STAT3 a known activator of c-MYC by antisense oligonucleotide AZD9150 (Hong 2015) inhibits survival of A431 epidermis cancer cells in vitro and in vivo. C-MYC overexpression is known to drive melanoma metastasis Lin (2017).

### Oral Squamous Cells Carcinoma.

knockdown of c-MYC in ORL-136T resulted in a significant decrease in the cell viability and c-MYC protein synthesis. Furthermore, caspase-3 was shown to be upregulated leading to apoptosis via the intrinsic pathway (Sabit 2021).

### Haemopoietic Cancers.

K562 cells treated with antisense oligodeoxynucleotides (ASOs) designed to bind to and inhibit a target mRNA showed a concentration-dependent decrease in both c-MYC mRNA and protein levels, with a maximal 65% inhibition of protein expression and inhibited cell growth by 70% (Coulis 2000). Small molecule c-MYC inhibitor 10058-F4 reduced viability in acute promyelocytic leukemia (APL)-derived NB4 cells (with wild-type PTEN) and acute lymphoblastic leukemia (ALL)-derived Nalm-6 cells (with down-regulated PTEN). In the presence of a PI3K inhibitor BKM120, 10058-F4 had significantly increased potency (Bashash 2019). Vitamin K2 and cotylenin A synergistically induce monocytic differentiation and growth arrest along with the suppression of c-MYC expression and induction of cyclin G2 expression in human leukemia HL-60 cell (Maniwa 2015). Inhibition of STAT3, a known activator of c-MYC, by antisense oligonucleotide AZD9150 inhibits survival of SUP-M2 tumor mice models (Hong 2015).

### Neuroblastoma.

CSNK1e is synthetic lethal with n-MYC in neuroblastomas. Using RNAi and small-molecule inhibitors, they confirmed that inhibition of CSNK1e halted growth of n-MYC-amplified neuroblastoma xenografts (Toyoshima 2012). These authors further show that CSNK1e expression was also associated with c-MYC in other tumor settings such as colon, lung, and breast cancer. An independently derived link between c-MYC and CSNK1e was obtained through the genome-wide identification of modifiers of transcription factor activity through the MINDy algorithm, where CSNK1e scored as modifier of c-MYC target gene expression (Wang 2009)

Taken together, these and other studies support the hypothesis that overexpression of c-MYC or n-MYC can play a causative role in many human cancers and that small molecule agents that block translation of c-MYC or n-MYC can be expected to provide therapeutic benefit.

c-MYC Translation Inhibitors May Synergize with Other Known Anti-Cancer Drugs.

In another aspect, described herein are methods of treating cancer, the method comprising administering to a subject in need thereof an effective amount of an agent that modulates c-MYC and a second anti-cancer agent.

In some embodiments, the second anti-cancer agent is chosen from Quizartinib, Gilteritinib, Venetoclax, Osimertinib, Crizotinib, Irinotecan, and Olaparib.

Synergy with PARP Inhibitors

In some embodiments the second anti-cancer agent is chosen from PARP inhibitors, immune oncology (I/O) drugs, and tyrosine kinase inhibitors.

A number of lines of evidence show that c-MYC expression and nuclear poly(ADP-ribose) polymerase (PARP) activity are closely related suggesting that c-MYC inhibitors could potentially work synergistically with PARP inhibitors. Fekete et al (2012) show that the human PARP-1 protein participates in the regulation of the c-MYC gene expression through its interaction with the Guanine-Quadruplex (GQ) structure of the human c-MYC gene promoter in vitro and in vivo. The c-MYC/MAX heterodimer binds directly to the PARP1 promoter and mediates transcription; high c-MYC expression correlates to PARP1 sensitivity in multiple myeloma (MM) (Caraciollo 2021) Burkitt lymphoma (Muvarak 2015) and neuroblastomas (Colicchia 2017).

Caraciollo et al (2021) show that high c-MYC expression correlates with sensitivity to PARP inhibitors in multiple myeloma. They found a significant correlation between higher PARP1 mRNA expression and poor prognosis of MM patients. PARP1 knockdown or its pharmacological inhibition by olaparib impaired MM cell viability in vitro and was effective against in vivo xenografts of human MM. They identified c-MYC as a promoter of PARP1-mediated repair in MM and demonstrated that cytotoxic effects induced by PARP inhibition are mostly detectable in c-MYC-proficient MM cells. Their findings show that c-MYC-driven MM cells are addicted to PARP1 alternative non-homologous end joining repair (Alt NHEJ).

Leukemias expressing the constitutively activated tyrosine kinases (TKs) BCR-ABL1 and FLT3/ITD activate signaling pathways that increase genomic instability through generation of reactive oxygen species (ROS), DNA double-strand breaks (DSBs) and error-prone repair. In TK-activated-leukemia Mavurak et al (2015) show that c-MYC expression leads to genomic instability via augmented expression of an alternative form of NHEJ (ALT-NHEJ), evidenced by increased expression of DNA ligase IIIα (LIG3) and poly (ADP-ribose) polymerase (PARP1).

In c-MYC driven B-cell lymphomas Galindo Campos et al (2022) show that selective PARP-2 inhibition prevents lymphoma progression, potentially representing a new therapeutic approach for the treatment of c-MYC-driven tumors.

c-MYC Translation Inhibitors May Synergize with Immune Oncology (I/O) Drugs

Dhanasekaran et al (2022) review evidence that c-MYC pathways not only govern cancer cell pathophysiology, but also play a key role in suppressing the ability of the host immune system to attack cancer cells. Among these mechanisms is the governance of PDL1 expression outlined below.

Han et al (2019) using small molecule MYC/MAX/DNA inhibitors (MYCi) show that it induces immunogenic cell death (ICD) in tumor cells and allows increased T cell infiltration and subsequent upregulation of PD-L1 in the tumor microenvironment. Accordingly, MYCi treatment sensitized otherwise refractory tumors to immune checkpoint blockade. A possible treatment regimen is one in which a c-MYC inhibitor is given to patients for a limited period of time prior to, concurrently with or followed by immune checkpoint blockade, thus avoiding potential toxicities to normal tissues that may arise from prolonged c-MYC inhibition.

Immunotherapy using anti-PD-1/PD-L1 antibodies has been shown to be effective for many types of malignancies however its activity has been limited in Human pancreatic ductal adenocarcinoma (PDAC). Pan et al (2019) have shown using several PDAC cell lines a significant correlation between the expression of PD-L1 and c-MYC. Further they show that JQ1, an inhibitor of the c-MYC pathway (acting via BRD4) inhibited PD-L1 expression and tumor growth. Using xenograft models, Pan et al demonstrated that the combination of JQ1 and anti-PD-L1 antibody exerted synergistic inhibition of PDAC growth. This is further supported by the work of Casey et al (2018) who provide evidence that c-MYC regulates expression of PD-L1 and that c-MYC translation inhibitors should therefore lower PD-L1 levels in tumors and may provide clinical benefit by augmenting I/O therapeutic regimens.

Taken together, this literature suggests that treatment of patients with c-MYC translation inhibitors may reverse cancerous growth and may augment or restore host antitumor immune response in patients with c-MYC-driven cancers (Dhanasekaran et al, 2022).

c-MYC Translation Inhibitors May Synergize with Tyrosine Kinase Inhibitors

Overexpression of c-MYC has been hypothesized to drive tyrosine kinase resistance. For example, Iyer (2022) show that in c-MYC-amplified patient-derived cell line and xenograft (LUAD-0006) c-MYC overexpression induces broad ROS1-TKI resistance. Pharmacologic inhibition of ROS1 combined with c-MYC knockdown were essential to completely suppress LUAD-0006 cell proliferation compared with either treatment alone.

Simertinib (AZD9291 or TAGRISSO™) is a promising and approved third-generation epidermal growth factor receptor (EGFR) tyrosine kinase inhibitor (TKI) for treating patients with advanced non-small cell lung cancer (NSCLC) harboring EGFR-activating mutations or the resistant T790M mutation. Zhu et al (2021) show that the mechanism of action of Osimertinib appears to be mediated via rapid and sustained decrease of c-MYC levels primarily via enhancing protein degradation in EGFR-mutant (EGFRm) NSCLC cell lines and xenograft tumors. In fact, c-MYC levels were substantially elevated in different EGFRm NSCLC cell lines with acquired resistance to osimertinib in comparison with their corresponding parental cell lines and could not be reduced any further by osimertinib. Thus, c-MYC translation inhibitors may well provide clinical benefit in combination with these EGFR inhibitors.

ALK tyrosine kinase inhibitors (TKIs) are the standard treatment for advanced ALK-positive NSCLC. Nevertheless, drug resistance inevitably occurs. Minari et al (2022) show the case of a patient with metastatic ALK positive lung adenocarcinoma with resistance to sequential treatment with ALK TKIs (7 months of crizotinib, brigatinib and lorlatinib) mediated by YES1 and c-MYC amplification.

Synthetic Lethal Interactions with c-MYC Suggest Other Combination Therapies

A phenomenon of c-MYC mediated synthetic lethality has been observed with various targets, including CDK1 cyclin-dependent kinase 1, CHK1 checkpoint kinase 1, GLS glutaminase (Chen 2018). Inhibitors of any of these targets could work in concert with inhibition of c-MYC translation.

Genome-wide RNA interference screens to search for c-MYC-synthetic lethal genes uncovered a role for the SUMO-activating enzyme (SAE1/2). In vitro and in vivo data (Kessler 2012) in SUM159 and MDA-MB-231 show that loss of SAE1/2 enzymatic activity drives synthetic lethality with c-MYC.

Hsu et al (2015) identify BUD31, a component of the core spliceosome, as a c-MYC-synthetic lethal gene. Core splice-osomal factors (SF3B1, U2AF1, and others) associated with BUD31 are also required to tolerate oncogenic c-MYC. Notably, c-MYC hyperactivation induces an increase in total pre-mRNA synthesis, suggesting an increased burden on the core spliceosome to process pre-mRNA. Genetic or pharmacologic inhibition of the spliceosome in vivo impairs survival, tumorigenicity, and metastatic proclivity of c-MYC-dependent breast cancers.

Toyoshima et al (2012) identified synthetic lethal interactions with c-MYC overexpression (MYC-SL) in a collection of ˜3,300 druggable genes, using high-throughput siRNA screening. Among 49 genes selected for follow-up, 48 were confirmed by independent retesting and approximately one-third selectively induced accumulation of DNA damage, consistent with enrichment in DNA-repair genes by functional annotation. In addition, genes involved in histone acetylation and transcriptional elongation, such as TRRAP and BRD4, were identified, indicating that the screen revealed known c-MYC-associated pathways.

Taken together, these studies support a rationale for combining inhibitors of c-MYC translation with inhibitors of one or more of the pathways given in Table 3.

TABLE 3
Genes and pathways for which there is evidence
of synthetic lethality with c-MYC

| Gene or pathway | Comment | Reference |
|---|---|---|
| PARP-1 | Commercially available | Fekete et al (2012) |
| | drugs | |
| PDL-1 | Commercially available | Han et al (2019) |
| | drugs | |
| EGFR | Commercially available | Zhu et al (2021) |
| | drugs | |
| ALK | Commercially available | Minari et al (2022) |
| | drugs | |
| CDK1 cyclin- | | Chen et al 2018 |
| dependent | | |
| kinase 1 | | |
| CHK1 checkpoint | | Chen et al 2018 |
| kinase 1, | | |
| GLS glutaminase | | Chen et al 2018 |
| SAE1/2 | | Kessler et al 2012 |
| BUD31 | | Hsu et al (2015) |
| TRRAP | | Toyoshima et al (2012) |
| BRD4 | | Toyoshima et al (2012) |
| Genes involved with | | Toyoshima et al (2012) |
| DNA damage repair | | |
| genes involved in | | Toyoshima et al (2012) |
| histone acetylation | | |
| and transcriptional | | |
| elongation | | |
| Core splice-osomal | | Hsu et al (2015) |
| factors (SF3B1, | | |
| U2AF1 | | |
| tyrosine kinase | | Iyer et al (2022) |
| resistance | | |

It is well known that c-MYC genes are artificially amplified in copy number due to the encoding genes being present on aberrant extrachromosomal elements that occur in elevated copy number in the affected cancer cells. Cancer patients for whom extrachromosomal amplification of c-MYC-encoding DNA is confirmed may be preferred patients for treatment with c-MYC inhibitors and the presence of such extrachromosomal elements may provide the basis for selecting patients for therapy. Diagnostics for circular extrachromosomal elements encoding c-MYC may also serve as an informative biomarker. There are well established methods for enriching for circular DNA relative to linear genomic DNA. For example, Moller et al (2016) report genome-wide methods for selectively purifying and amplifying circular eukaryotic DNA.

Biomarkers for c-MYC Inhibition

c-MYC drives the transcriptional regulation of a number of genes, some of which could be used as biomarkers for on-target activity or as predictors of anti-c-MYC activity. Zeller et al (2003) report a database of genes responsive to the c-MYC oncogenic transcription factor. The database c-MYC Target Gene prioritizes candidate target genes according to experimental evidence and clusters responsive genes into functional groups. Using mRNA profiling, Perna et al (2012) identify close to 300 c-MYC-dependent serum response (MDSR) genes, which are induced by serum in a c-MYC-dependent manner in mouse fibroblasts. MDSR gene products were involved in a variety of processes, including nucleotide biosynthesis, ribosome biogenesis, DNA replication and RNA control. To delineate the molecular program regulated downstream of c-MYC, Watson et al (2002) used a cDNA microarray approach and identified 52 putative targets out of >6000 cDNAs analyzed. The list was further narrowed down to c-MYC specific target genes (see table below) rather than genes regulated in response to mitogenic or apoptosis events.

Differentially expressed genes from our microarray analysis that fulfill the criteria of a Myc-regulated target gene

| | | Regulated in the |
|---|---|---|
| Gene target | Description | presence of cyc [symbol] |
| Activated | | |
| APEX | Multifunctional DNA repair enzyme | NI |
| KIAA0664 | Unknown | / |
| Nucl [symbol] (NCL) | RNA-binding nucleolar phosphoprotein | NI |
| Nucleoph [symbol] smin (NPM [symbol] ) | RNA-binding nucleolar phosphoprotein | NI |
| TOB3 (FLJ10709) | Has low similarity to SPG7 (paraplegin); member of the AAA ATPase | NI |
| | protein family | |
| Repressed | | |
| Cathepsin B (CTSB) | Lysosomal cysteine protease | NI |
| Δ6 fatty acid des [symbol] (FADS2) | Catalyzes production of polyenoic fatty acids | NI |
| Fibronectin (FN1) | Member of the family of proteins found in plasma and extracellular | NI |
| | matrix | |
| LAMP2 | Glycoprotein that provides selectins with carbohydrate ligands | NI |
| N-PAC | Cytokine-like nuclear factor | NI |
| pdgfβr (PDGFB) | Platelet-derived growth factor receptor beta chain | NI |
| Similarity to serine carboxypeptidase 1 | Lysosomal glycoprotein | / |
| Thymosin [symbol] (TMSB- [symbol] X) | Sequesters actin monomers and inhibits actin polymerization | NI |
| TM [symbol] -1 (TDE1) | Tumor differentially expressed | NI |
| TRIP12 | May have ubiquitin-protein ligase activity | NI |

[symbol]

j [symbol] result is indicative of a direct Myc target.
NI = not informative [symbol]
indicates data missing or illegible when filed

### V. REFERENCES

1. Bashash D. et al (2019) IJBCB, 108:7.
2. Bouvard C. et al (2017) PNAS, 114(13): 3497.
3. Caracciolo D. et al (2021) Haematologica, 106(1): 185.
4. Carey J. P. W. et al (2018) Cancer Res., 78(3): 742
5. Casey et al (2018) Blood, 131:2007.
6. Chen H et al (2018) Signal Transduction and Targeted Therapy, 3:5.
7. Colicchia V. et al (2017) Oncogene, 4682.
8. Coulis C. M. et al (2000) Mol Phmarcol, 57:485.
9. Dang C. V. et al (2012) Cell, 149(1): 22.
10. Delmore J. E. (2011) Cell, 146(6): 904.
11. Deracinois B. et al. (2013) Proteomes, 1, 180-218.
12. Dhanasekaran R et al (2020) Molecular Therapy: Nucleic Acids, 21:850.
13. Dhanasekaran R et al (2022) Nature Reviews, 19:23.
14. Fekete A. et al (2012) PlosOne, 7(8): e42690.
15. Felsenstein K. M. et al (2016) ACS Chemical Biology, 11:139.
16. Felsher D. W. and Bishop M. (1999) Molecular Cell, 4:199.
17. Felsher D. W. (2010) Genes and Cancer, 1(6): 597.
18. Galindo-Campos et al (2022) Blood, 139(2): 228.
19. Hahm E. (2016) Cell Cycle, 15(17): 2309.
20. Han H. (2019) Cancer Cell, 36(5): 483.
21. Hong D. et al (2015) Sci Transl Med., 7(314).
22. Horiuchi D. et al (2015) J Expt Med., 209(4): 679.
23. Hsu T. Y. (2015) Nature, 525(7569): 384.
24. Iyer S. R. (2022) Mol Cancer Res, 20(5): 722.
25. Kang J. et al (2019) Laboratory Animal Research, 35:30.
26. Kessler J. D. et al (2012) Science, 335:348.
27. Lin X. et al (2017) J Mol Med, 95:53.
28. Liombart et al (2022) The Lancet, 75:103756.
29. Liu X. et al (2020) Frontiers in Pharmacology, 11(495).
30. Madden S. K. et al (2021) Molecular Cancer, 20:3.
31. Maniwa Y. et al (2015) Int J Oncology, 47:473.
32. Mikami S. et al (2010) in Chapter 5 of “Cell Free Protein Production”, Humana Press New York: 43.
33. Minara R. et al (2022) JTO Clinical and Res Reports, 3(2): 100278.
34. Moller H. D. et al (2016) J Vis Exp. 110:54239.
35. Muvarak N. et al (2015) Mol Cancer Res, 13(4): 699.
36. Pan Y. et al (2019) Oncoimmunology, 8(5): e1581529.
37. Perna D. et al (2012) Oncogene, 31:1695.
38. Raunivar N. et al. (2014) J Proteome Research, 13, 5293-5309.
39. Sabit H. et al (2021) IJMCM, 20(1): 45.
40. Schumacher M. et al (1999) Current Biology, 9:1255.
41. Shachaf C. M. (2004) Nature, 431:1112.
42. Shroff E. H. (2015) PNAS, 112(21): 6539.
43. Soucek L. (2008) Nature, 455:679.
44. Sriratanasak N (2020) Mol Pharmacol, 98:130.
45 Steinberger J. et al (2019) Cell Chemical Biology, 26:699.
46. Toyoshima M. et al (2012) PNAS, 109(24): 9545.
47. Turner K. M. et al (2017) Nature, 543(7643): 122.
48. Wang Y. et al (2004) Breast Cancer Research, 7(2): R220.
49. Wang K. et al (2009) Nat Biotechnol, 27(9): 829.
50. Watson J. D. (2002) J Biol Chem, 277(4): 36921.
51. Zhu L. et al (2021) Cancer Res, 81(18): 4822.
52. Zeller K. I. (2003) Genome Biology, 4: R69.

Taken together, the literature cited above supports the utility of embodiments disclosed herein, in addition to, but not limited to, kits to measure protein or mRNA levels in the noted pathways as either diagnostics to select patients for therapy or as companion diagnostics to assess efficacy of c-MYC translation inhibitors in a clinical setting.

The disclosure further provides any compounds disclosed herein for use in a method of treatment of the human or animal body by therapy. Therapy may be by any mechanism disclosed herein, such as inhibiting, reducing, or reducing progression of the diseases disclosed herein. The disclosure further provides any compound disclosed herein for prevention or treatment of any condition disclosed herein. The disclosure also provides any compound or pharmaceutical composition thereof disclosed herein for obtaining any clinical outcome disclosed herein for any condition disclosed herein. The disclosure also provides use of any compound disclosed herein in the manufacture of a medicament for preventing or treating any disease or condition disclosed herein.

### VI. EMBODIMENTS

Provided herein are various embodiments of the invention. The following embodiments provide additional non-limiting examples of the present invention.

Embodiment A1 is a method of modulating c-MYC activity comprising administering an effective amount of a compound of Formula I

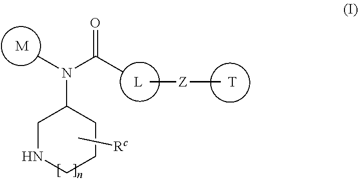

wherein
M is C₁₋₉ heteroaryl, C₁₋₉ heterocyclyl, each optionally substituted with 1, 2 or 3 substituents chosen from halo, alkyl, alkenyl, alkynyl, aryl, ester, carboxylic acid, amide, amino, alkoxy, thioether, amino, cyano, heteroaryl, cycloalkyl, heterocyclyl, and combinations thereof;
R^{c} is H, C₁-C₆ alkyl, OH, NH₂, fluoro or gem-difluoro;
n is 0, 1 or 2;
L is a bond, C₆₋₁₀ arylene, C₃₋₉ heteroarylene, C₃₋₁₀ cycloalkylene, or C₁₋₉ heterocyclylene, each optionally substituted with 1, 2 or 3 substituents chosen from halo, aryl, heteroaryl, alkyl, alkenyl, alkynyl, cycloalkyl, heterocyclyl, carboxylic acid, ester, amide, alkoxy, alkoxyaryl, alkoxyheteroaryl, thioether, amino, NH-aryl, NH-heteroaryl, and cyano;
Z is a bond, —O—, C₁₋₃ alkylene, —N(R^{b})—, where R^{b} is H, or C₁₋₃ alkyl; and
T is absent, C₆₋₁₀ aryl, C₃₋₁₀ cycloalkyl, C₃₋₉ heteroaryl, C₁₋₉ heterocyclyl, each optionally substituted with 1, 2 or 3 substituents chosen from halo, aryl, heteroaryl, alkyl, alkenyl, alkynyl, cycloalkyl, heterocyclyl, carboxylic acid, ester, amide, alkoxy, alkoxyaryl, alkoxyheteroaryl, thioether, amino, NH-aryl, NH-heteroaryl, and cyano;
provided that when L is a bond, T is not absent and when T is absent, then L is not a bond; or a pharmaceutically acceptable salt thereof.

Embodiment A2 is the method of Embodiment A1, wherein the MYC activity is modulated in a cell.

Embodiment A3 is the method of Embodiment A1, wherein the MYC activity is modulated in a cellular assay.

Embodiment A4 is the method of Embodiment A1, wherein the MYC activity is modulated in an In Vitro Multiple Point Translation Screening Assay.

Embodiment A5 is the method of Embodiment A1, wherein the MYC activity is modulated in a subject in need thereof, wherein the subject is a model animal or a human.

Embodiment A6 is the method of Embodiment A1, wherein the MYC activity modulates transcriptional regulation of a gene that is a biomarker.

Embodiment A7 is the method of Embodiment A1, wherein the MYC activity modulates transcriptional regulation of a gene that is a biomarker that predicts anti-MYC activity.

Embodiment A8 is the method of anyone of Embodiments A1-7, wherein the compound of Formula I inhibits MYC activity.

Embodiment A9 is the method of any one of Embodiments A1 to 7, wherein the compound of Formula I activates MYC activity.

Embodiment A10 is a method of treating a condition associated with MYC activity in a subject in need thereof comprising administering to the subject an effective amount of a compound of Formula I

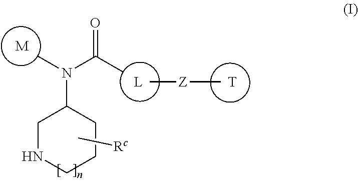

wherein
M is C₁₋₉ heteroaryl, C₁₋₉ heterocyclyl, each optionally substituted with 1, 2 or 3 substituents chosen from halo, alkyl, alkenyl, alkynyl, aryl, ester, carboxylic acid, amide, amino, alkoxy, thioether, amino, cyano, heteroaryl, cycloalkyl, heterocyclyl, and combinations thereof;
n is 0, 1 or 2;
R^{c} is H, C₁-C₆ alkyl, OH, NH₂, fluoro or gem-difluoro;
L is a bond, C₆₋₁₀ arylene, C₃₋₉ heteroarylene, C₃₋₁₀ cycloalkylene, or C₁₋₉ heterocyclylene, each optionally substituted with 1, 2 or 3 substituents chosen from halo, aryl, heteroaryl, alkyl, alkenyl, alkynyl, cycloalkyl, heterocyclyl, carboxylic acid, ester, amide, alkoxy, alkoxyaryl, alkoxyheteroaryl, thioether, amino, NH-aryl, NH-heteroaryl, and cyano;
Z is a bond, —O—, C₁₋₃ alkylene, —N(R^{b})—, where R^{b} is H, or C₁₋₃ alkyl; and
T is absent, C₆₋₁₀ aryl, C₃₋₁₀ cycloalkyl, C₃₋₉ heteroaryl, C₁₋₉ heterocyclyl, each optionally substituted with 1, 2 or 3 substituents chosen from halo, aryl, heteroaryl, alkyl, alkenyl, alkynyl, cycloalkyl, heterocyclyl, carboxylic acid, ester, amide, alkoxy, alkoxyaryl, alkoxyheteroaryl, thioether, amino, NH-aryl, NH-heteroaryl, and cyano;

provided that when L is a bond, T is not absent and when T is absent, then L is not a bond;

or a pharmaceutically acceptable salt thereof.

Embodiment A11 is the method of any one of Embodiments A1 to 10, wherein the condition associated with MYC activity is cancer.

Embodiment A12 is the method of Embodiment A11, wherein the cancer is chosen from solid and hematopoietic cancers such as breast, prostate, pancreatic, hepatocellular, renal, multiple myeloma, lymphoma, lung, skin, oral squamous, haemopoietic, and neuroblastoma.

Embodiment A13 is the method of Embodiment A11, wherein the cancer is derived from breast, colon, lung or hematopoietic lineages.

Embodiment A14 is the method of any one of Embodiments A11 to 13, wherein the method further comprises administering to the subject an effective amount of a second anti-cancer agent.

Embodiment A15 is the method of Embodiment A14, wherein the second anti-cancer agent is chosen from PARP inhibitors, immune oncology (I/O) drugs, tyrosine kinase inhibitors, CHK1 inhibitor, GLS inhibitor, SAE1/2 modulators, BUD31 modulators, TRRAP modulators and BRD4 modulators.

Embodiment A16 is the method of Embodiment A14, wherein the second anti-cancer agent is chosen from Olaparib (Lynparza), rucaparib (Rubraca), niraparib (Zejula), nivolumab (Opdivo), atezolizumab (Tecentriq), pembrolizumab (Keytruda), avelumab (Bavencio), durvalumab (Imfinzi) and cemiplimab-rwlc (Libtayo), axitinib (Inlyta) dasatinib (Sprycel), erlotinib (Tarceva), imatinib (Glivec), nilotinib (Tasigna), pazopanib (Votrient), sunitinib (Sutent).

Embodiment A17 is the method of any one of Embodiments A1 to 16, wherein M is pyrrolo[2,3-c]pyridinyl, pyrrolo[3,2-c]pyridinyl, quinolinyl, isoquinolinyl, pyridinyl, or 2-pyrazinyl, each optionally substituted with 1 or 2 substituents chosen from halo, C₁₋₂ alkyl and combinations thereof.

Embodiment A18 is the method of any one of Embodiments A1 to 17, wherein M is chosen from 1-methyl-3H-pyrrolo[2,3-c]pyridin-7-yl, 1-methyl-3H-pyrrolo[2,3-c]pyridin-5-yl, 1-methyl-3H-pyrrolo[3,2-c]pyridin-6-yl, quinolin-2-yl, isoquinolinyl, isoquinolin-3-yl, 8-methylisoquinolinyl, pyridin-2-yl, 3-chloropyridin-2-yl, 3-methylpyridin-2-yl, and 2-methylpyrazin-3-yl.

Embodiment A19 is the method of any one of Embodiments A1 to 16, wherein M is

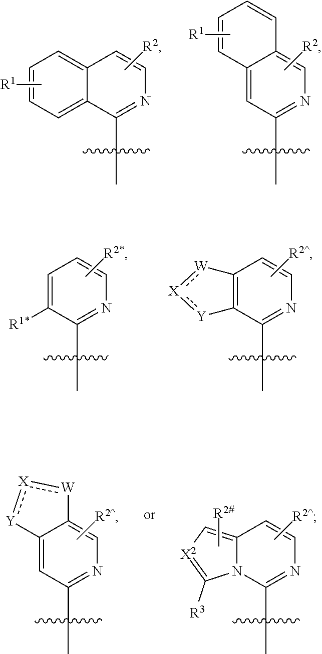

wherein
R¹ is H, halo, alkyl, alkenyl, alkynyl, aryl, ester, carboxylic acid, amide, alkoxy, thioether, amino, cyano, heteroaryl, cycloalkyl or heterocyclyl;
R² is H, halo, alkyl, alkenyl, alkynyl, aryl heteroaryl or cyano;
R^{1*} is H, halo, alkyl, alkenyl, alkynyl, aryl, ester, carboxylic acid, amide, alkoxy, thioether, cyano, heteroaryl, cycloalkyl or heterocyclyl;
R^{2*} is H, halo, alkyl, alkenyl, alkynyl, aryl, ester, carboxylic acid, amide, alkoxy, thioether, cyano, heteroaryl, cycloalkyl or heterocyclyl; and
each W, X, and Y are independently —CH, —N, —NH, N-alkyl, O, or S;
R^{2#} is H, halo, alkyl, alkenyl, alkynyl, aryl, ester, carboxylic acid, amide, alkoxy, thioether, amino, cyano, heteroaryl, cycloalkyl or heterocyclyl;
X² is CH, N, C-alkyl, C-alkenyl, C-alkynyl, C-aryl or C-heteroaryl;
R^{2\hat{ }} is H, alkyl, alkenyl, alkynyl, cyano or halo; and
R³ is H, alkyl, alkenyl, alkynyl, halo, cyano, ester, carboxylic acid, amide, aryl, heteroaryl, alkoxy, thioether, or amino; and
[symbol] is a bond that is a double bond or a single bond.

Embodiment A20 is the method of any one of Embodiments A1 to 16, wherein M is

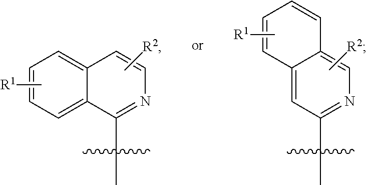

wherein
R¹ is H, halo, alkyl, alkenyl, alkynyl, aryl, ester, carboxylic acid, amide, alkoxy, thioether, amino, cyano, heteroaryl, cycloalkyl or heterocyclyl; and
R² is H, halo, alkyl, alkenyl, alkynyl, aryl heteroaryl or cyano.

Embodiment A21 is the method of any one of Embodiments A1 to 16, wherein M is

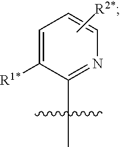

wherein
R^{1*} is H, halo, alkyl, alkenyl, alkynyl, aryl, ester, carboxylic acid, amide, alkoxy, thioether, cyano, heteroaryl, cycloalkyl or heterocyclyl; and
R^{2*} is H, halo, alkyl, alkenyl, alkynyl, aryl, ester, carboxylic acid, amide, alkoxy, thioether, cyano, heteroaryl, cycloalkyl or heterocyclyl.

Embodiment A22 is the method of any one of Embodiments A1 to 16, wherein M is

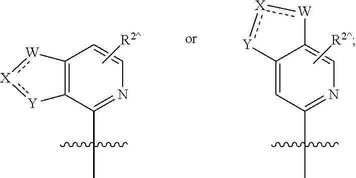

wherein
each W, X, and Y are independently —CH, —N, —NH, N-alkyl, O, or S;
R^{2\hat{ }} is H, halo, alkyl, alkenyl, alkynyl, aryl, ester, carboxylic acid, amide, alkoxy, thioether, amino, cyano, heteroaryl, cycloalkyl or heterocyclyl; and
[symbol] is a bond that is a double bond or a single bond.

Embodiment A23 is the method of any one of Embodiments A1 to 16, wherein M is

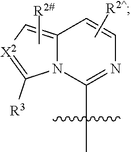

wherein
X² is CH, N, C-alkyl, C-alkenyl, C-alkynyl, C-aryl or C-heteroaryl;
R^{2#} is H, halo, alkyl, alkenyl, alkynyl, aryl, ester, carboxylic acid, amide, alkoxy, thioether, amino, cyano, heteroaryl, cycloalkyl or heterocyclyl;
R^{2\hat{ }} is H, alkyl, alkenyl, alkynyl, cyano or halo; and
R³ is H, alkyl, alkenyl, alkynyl, halo, cyano, ester, carboxylic acid, amide, aryl, heteroaryl, alkoxy, thioether, or amino.

Embodiment A24 is the method of any one of Embodiments A1 to 23, wherein L is a bond, phenylene, pyridinylene, piperidinylene, or piperazinylene, each optionally substituted with 1 or 2 substituents chosen from halo, C₁₋₃ alkyl, C₁₋₃ alkyloxy and combinations thereof, or —NR^{a}—, where R^{a} is H or C₁₋₃ alkyl.

Embodiment A25 is the method of any one of Embodiments A1 to 23, wherein L is a bond.

Embodiment A26 is the method of any one of Embodiments A1 to 23, wherein L is phenylene, pyridinylene, piperidinylene, or piperazinylene, each optionally substituted with 1 or 2 substituents chosen from halo, C₁₋₃ alkyl, C₁₋₃ alkyloxy and combinations thereof.

Embodiment A27 is the method of any one of Embodiments A1 to 23, wherein L is chosen from phenylene, fluorophenylene, chlorophenylene, methoxyphenylene, methylphenylene, ethylphenylene, pyridinylene, piperidinylene, and piperazinylene.

Embodiment A28 is the method of any one of Embodiments A1 to 23, wherein L is —NR^{a}—, where R^{a} is H or C₁₋₃ alkyl.

Embodiment A29 is the method of any one of Embodiments A1 to 23, wherein L is-NR^{a}—, where R^{a} is H, isopropyl, or methyl.

Embodiment A30 is the method of any one of Embodiments A1 to 29, wherein Z is a bond.

Embodiment A31 is the method of any one of Embodiments A1 to 29, wherein Z is C₁₋₃ alkylene.

Embodiment A32 is the method of any one of Embodiments A1 to 29, wherein Z is methylene.

Embodiment A33 is the method of any one of Embodiments A1 to 29, wherein Z is —NR^{b}—, where R^{b} is H, or C₁₋₃ alkyl.

Embodiment A34 is the method of any one of Embodiments A1 to 29, wherein Z is —NR^{b}—, where R^{b} is H or methyl.

Embodiment A35 is the method of any one of Embodiments A1 to 34, wherein T is [1,2,3]triazolo[4,5-b]pyridinyl, pyrazolo[2,3-a]pyrimidinyl, 5,6-dihydropyrrolo[2,3-d]pyrimidinyl, phenyl, purinyl, pyrimidinyl, pyridinyl, pyridinyl, pyrazolyl, tetrazolyl, 1,2,4-triazolyl, 1,2,3-triazolyl, imidazolyl, benzo[d]imidazolyl, pyrrolyl, isoxazolyl, isoindolyl, piperidinyl, or oxetanyl each optionally substituted with 1 or 2 substituents chosen from Cl, F, cyano, C₁₋₃ alkyl, C₁₋₃ alkyloxy and combinations thereof.

Embodiment A36 is the method of any one of Embodiments A1 to 34, wherein T is chosen from [1,2,3]triazolo[4,5-b]pyridin-3-yl, 5-methyl-1H-[1,2,3]triazolo[4,5-b]pyridin-3-yl, 1H-[1,2,3]triazolo[4,5-c]pyridin-4-yl, pyrazolo[2,3-a]pyrimidin-3-yl, 5,6-dihydro-1H-pyrrolo[2,3-d]pyrimidin-4-yl, phenyl, 2-cyanophenyl, 3-cyanophenyl, 3-cyano-6-fluorophenyl, 4-cyanophenyl, 4-methoxyphenyl, purin-6-yl, pyrimidin-2-yl, pyrimidin-5-yl, 4-cyanopyrimidin-2-yl, 2-chloropyridin-6-yl, 4-chloropyridin-6-yl, pyridin-2-yl, pyridin-3-yl, pyridin-4-yl, 2-cyanopyrazin-6-yl, 1-methyl-1H-pyrazol-5-yl, 1-methyl-1H-pyrazol-4-yl, 5-methyl-1H-pyrazolyl, tetrazol-2-yl, 4H-1,2,4-triazol-4-yl, 1H-1,2,3-triazolyl, 1H-1,2,3-triazol-2-yl, 1H-imidazolyl, 2-methyl-benzo[d]imidazolyl, pyrrolyl, isoxazol-5-yl, 3-methylisoxazol-5-yl, 5-cyano-1H-isoindol-2-yl, 1-methylpiperidin-4-yl, and oxetan-3-yl.

Embodiment A37 is the method of any one of Embodiments A1 to 23, wherein ring L, Z and ring T together are

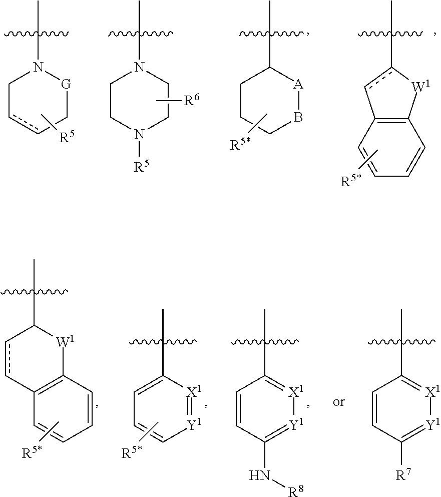

wherein
G is CH₂, CH-alkyl, NH, N-alkyl;
R⁵ is aryl, heteroaryl, alkyl, alkenyl, alkynyl, cycloalkyl, heterocyclyl, alkoxy, amino, cyano, carboxylic acid, ester or amide;
R⁶ is H, halo, alkyl, alkenyl, alkynyl, aryl, ester, carboxylic acid, amide, alkoxy, thioether, amino, cyano, heteroaryl, cycloalkyl or heterocyclyl;
A and B are each independently CH₂, O or NH;
W¹ is —CH₂, O or NH;
X¹ and Y¹ are each independently CH or N;
R^{5*} is halo, cyano, amide, alkenyl, alkynyl, aryl, heteroaryl, alkoxyaryl, alkoxyheteroaryl, NH-aryl, or NH-heteroaryl;
R⁷ is alkyl, alkenyl, alkynyl, cycloalkyl, heterocyclyl, aryl or heteroaryl;
R⁸ is H, alkyl, aryl, heteroaryl, or heterocyclyl; and
[symbol] is a bond that is a double bond or a single bond.

Embodiment A38 is the method of any one of Embodiments A1 to 23, wherein ring L, Z and ring T together are

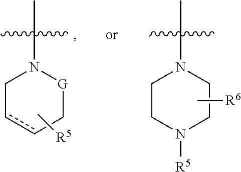

wherein
G is CH₂, CH-alkyl, NH, N-alkyl;
R⁵ is aryl, heteroaryl, alkyl, alkenyl, alkynyl, cycloalkyl, heterocyclyl, alkoxy, amino, cyano, carboxylic acid, ester or amide;
R⁶ is H, halo, alkyl, alkenyl, alkynyl, aryl, ester, carboxylic acid, amide, alkoxy, thioether, amino, cyano, heteroaryl, cycloalkyl or heterocyclyl; and
[symbol] is a bond that is a double bond or a single bond.

Embodiment A39 is the method of any one of Embodiments A1 to 23, wherein ring L, Z and ring T together are

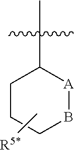

wherein
A and B are each independently CH₂, O or NH; and
R^{5*} is halo, cyano, amide, alkenyl, alkynyl, aryl, heteroaryl, alkoxyaryl, alkoxyheteroaryl, NH-aryl, or NH-heteroaryl.

Embodiment A40 is the method of any one of Embodiments A1 to 23, wherein ring L, Z and ring T together are

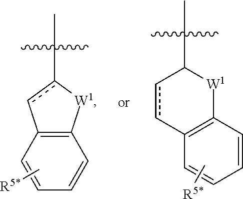

wherein
W¹ is —CH₂, O or NH;
R^{5*} is halo, cyano, amide, alkenyl, alkynyl, aryl, heteroaryl, alkoxyaryl, alkoxyheteroaryl, NH-aryl, or NH-heteroaryl; and
[symbol] is a bond that is a double bond or a single bond.

Embodiment A41 is the method of any one of Embodiments A1 to 23, wherein ring L, Z and ring T together are

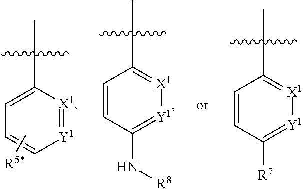

wherein
X¹ and Y¹ are each independently CH or N;
R^{5*} is halo, cyano, amide, alkenyl, alkynyl, aryl, heteroaryl, alkoxyaryl, alkoxyheteroaryl, NH-aryl, or NH-heteroaryl;
R⁷ is alkyl, alkenyl, alkynyl, cycloalkyl, heterocyclyl, aryl or heteroaryl; and
R⁸ is H, alkyl, aryl, heteroaryl, or heterocyclyl.

Embodiment A42 is the method of any one of Embodiments A1 to 41, wherein the compound is selected from Table 1, and/or a pharmaceutically acceptable.

Embodiment A43 is the method of any one of Embodiments A1 to 42, wherein the compound of Formula (I) is provided in a pharmaceutical composition comprising a compound and/or or a pharmaceutically acceptable salt thereof and a pharmaceutically acceptable excipient.

Embodiment A44 is a method of treating cancer by administering to a patient in need thereof a therapeutically effective amount of a compound that selectively inhibits the translation of c-MYC mRNA to c-MYC protein.

Embodiment A45 is the method of Embodiment A44, wherein translation stalls by targeting a cytosolic nascent chain/ribosome complex.

Embodiment A46 is the method of Embodiment A45, wherein a ternary complex between small molecule, ribosome and nascent c-MYC polypeptide is visible by Cryo-electron microscopy.

Embodiment A47 is the method of Embodiment A44, wherein translation is stalled to deliver 50% partial inhibition of translation.

Embodiment A48 is the method of Embodiment A45, wherein translation is stalled resulting in 90% partial inhibition of translation

Embodiment A49 is the method of Embodiment A44, wherein the compound has an IC50 below about 50 μM in an In Vitro Multiple Point Translation Screening Assay.

Embodiment A50 is the method of Embodiment A44, wherein the compound has an IC50 below about 5 μM in an In Vitro Multiple Point Translation Screening Assay.

Embodiment A51 is the method of Embodiment A44, wherein the selective inhibition of MYC is such that less than 1% of non-MYC proteins are inhibited in a Global Proteomic Assay.

Embodiment A52 is the method of Embodiment A44, wherein the selective inhibition of MYC is such that less than 0.1% of non-MYC proteins are inhibited in a Global Proteomic Assay.

Embodiment A53 is the method of Embodiment A43, wherein the compound is administered by oral administration.

Embodiment A54 is the method of Embodiment A43, wherein the compound has a MW of about 300 to about 600 Da.

Embodiment A55 is a compound of Formula (Ia)

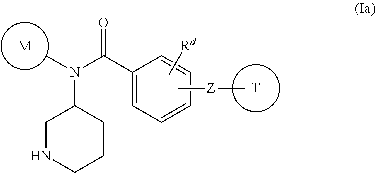

wherein
M is pyrrolo[3,2-c]pyridinyl, quinolinyl, or 2-pyrazinyl, each optionally substituted with 1 or 2 substituents chosen from halo, C₁₋₂ alkyl and combinations thereof;
R^{d} is halo, C₁₋₃ alkyl, or C₁₋₃ alkyloxy;
Z is a bond, C₁₋₃ alkylene, —N(R^{b})—, where R^{b} is H, or C₁₋₃ alkyl; and
T is 5,6-dihydropyrrolo[2,3-d]pyrimidinyl, purinyl, pyrimidinyl, pyridinyl, pyridinyl, tetrazolyl, 1,2,4-triazolyl, 1,2,3-triazolyl, imidazolyl, benzo[d]imidazolyl, pyrrolyl, isoxazolyl, isoindolyl, piperidinyl, or oxetanyl each optionally substituted with 1 or 2 substituents chosen from Cl, F, cyano, C₁₋₃ alkyl, C₁₋₃ alkyloxy and combinations thereof; or a pharmaceutically acceptable salt thereof.

Embodiment A56 is the compound of Embodiment A34, wherein R^{d} is Cl, F, CH₃, or OCH₃.

Embodiment A57 is a compound of Formula (Ib)

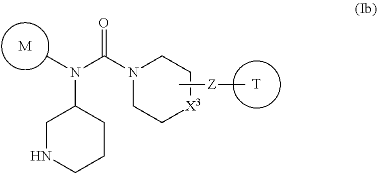

wherein
M is pyrrolo[2,3-c]pyridinyl, pyrrolo[3,2-c]pyridinyl, quinolinyl, or 2-pyrazinyl, each optionally substituted with 1 or 2 substituents chosen from halo, C₁₋₂ alkyl and combinations thereof;
X³ is CH₂ (or CH if bonded to T through Z), NH (or N if bonded to T through Z);
Z is a bond, C₁₋₃ alkylene, —N(R^{b})—, where R^{b} is H, or C₁₋₃ alkyl; and
T is [1,2,3]triazolo[4,5-b]pyridinyl, pyrazolo[2,3-a]pyrimidinyl, 5,6-dihydropyrrolo[2,3-d]pyrimidinyl, phenyl, purinyl, pyrimidinyl, pyridinyl, pyridinyl, pyrazolyl, tetrazolyl, 1,2,4-triazolyl, 1,2,3-triazolyl, imidazolyl, benzo[d]imidazolyl, pyrrolyl, isoxazolyl, isoindolyl, piperidinyl, or oxetanyl each optionally substituted with 1 or 2 substituents chosen from Cl, F, cyano, C₁₋₃ alkyl, C₁₋₃ alkyloxy and combinations thereof;
or a pharmaceutically acceptable salt thereof.

Embodiment A58 is the compound of Embodiment A36, wherein X is CH₂ (or CH if bonded to T through Z).

Embodiment A59 is the compound of Embodiment A36, wherein X is NH (or N if bonded to T through Z).

Embodiment A60 is the compound of any one of Embodiments A34 to 38, wherein Z is a bond or methylene.

Embodiment A61 is a compound of Formula (Ic)

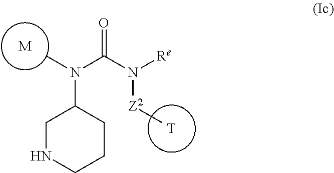

wherein
M is pyrrolo[2,3-c]pyridinyl, pyrrolo[3,2-c]pyridinyl, quinolinyl, isoquinolinyl, pyridinyl, or 2-pyrazinyl, each optionally substituted with 1 or 2 substituents chosen from halo, C₁₋₂ alkyl and combinations thereof;
R^{e} is H or C₁₋₃ alkyl;
Z² is a bond or C₁₋₃ alkylene; and
T is [1,2,3]triazolo[4,5-b]pyridinyl, pyrazolo[2,3-a]pyrimidinyl, 5,6-dihydropyrrolo[2,3-d]pyrimidinyl, phenyl, purinyl, pyrimidinyl, pyridinyl, pyridinyl, pyrazolyl, tetrazolyl, 1,2,4-triazolyl, 1,2,3-triazolyl, imidazolyl, benzo[d]imidazolyl, pyrrolyl, isoxazolyl, isoindolyl, piperidinyl, or oxetanyl each optionally substituted with 1 or 2 substituents chosen from Cl, F, cyano, C₁₋₃ alkyl, C₁₋₃ alkyloxy and combinations thereof;
or a pharmaceutically acceptable salt thereof.

Embodiment A62 is the compound of Embodiment A40, wherein R^{e} is H, methyl, or isopropyl.

Embodiment A63 is the compound of Embodiment A40 or 41, wherein Z² is a bond.

Embodiment A64 is the compound of Embodiment A40 or 41, wherein Z² is methylene.

Embodiment A65 is a compound selected from Table 1, and/or pharmaceutically acceptable salts thereof.

Embodiment A66 is a compound of any one of Embodiments A55 to 65, wherein one or more hydrogen atoms attached to carbon atoms of the compound are replaced by deuterium atoms.

Embodiment A67 is a pharmaceutical composition comprising a compound and/or a pharmaceutically acceptable salt or prodrug of any one of Embodiments A55 to 65 and a pharmaceutically acceptable excipient.

Embodiment A68 is a compound of any one of Embodiments A55 to 65 for use as a medicament.

Embodiment A69 is the compound of Embodiment A68, wherein the compound is for use in treating a cancer associated with MYC activity.

Embodiment A70 is the use of a compound of any one of Embodiments A55 to 65 in the manufacture of a medicament for treating a disease in a subject in which MYC contributes to the pathology and/or symptoms of the disease.

### EXAMPLES

In order that the invention described herein can be more fully understood, the following examples are set forth. It should be understood that these examples are included merely for purposes of illustration of certain aspects and embodiments of the present disclosure and are not to be construed as limiting the invention in any manner. Reasonable variations, such as those that occur to a reasonable artisan, can be made herein without departing from the scope of the present disclosure.

All chemicals, reagents, and solvents were purchased from commercial sources and used without further purification. Suzuki and Buchwald couplings were performed under an atmosphere of nitrogen. Nuclear magnetic resonance spectra (¹H NMR) were recorded with a 400 MHz Bruker spectrometer. Chemical shifts are expressed in parts per million downfield from tetramethylsilane. The peak shapes are denoted as follows: s, singlet; d, doublet; t, triplet; q, quartet; m, multiplet; br s, broad singlet. Due to differences in solvents used in sample preparation and, in some cases, amount of water present, not all exchangeable protons were observable. Liquid chromatograph-mass spectrometry (LC-MS) was performed on an Agilent 1200 HPLC/6100Series MSD/SEDEX 85 ELSD using an electron scatter ionization (ESI) source. Silica gel chromatography was performed using a medium pressure ISCO system using columns prepackaged by various commercial vendors including ISCO. Merck precoated silica gel plates (400 μm) were used for analytical TLC.

### 1.1 Experimental Details for Intermediate Synthesis

### Synthesis of 6 Het-Cl

### General Procedure for Preparation of Compound WA004

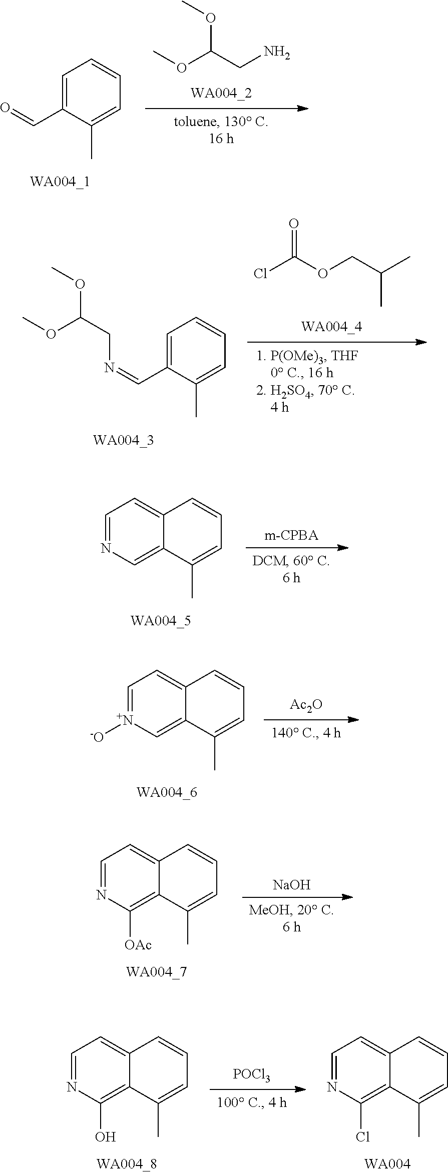

WA004_2 (87.5 g, 832 mmol) was added to a solution of compound WA004_1 (100 g, 832 mmol, 96.1 mL) in toluene (500 mL). The reaction mixture was stirred at 130° C. for 16 h. The reaction mixture was concentrated under reduced pressure to give a residue, which was used in the next step without further purification. Compound WA004_3 (120 g, 89%) was obtained as a pale yellow oil.

P(OMe)₃ (92.4 g, 744 mmol,) and compound WA004_4 (84.7 g, 620 mmol,) were added to a solution of compound WA004_3 (100 g, 620 mmol) in THF (300 mL). The reaction mixture was stirred at 0° C. for 16 h. Then, H₂SO₄ (20 mL) was added at 0° C., and the mixture was stirred at 70° C. for 4 h. The reaction mixture was diluted with water (50 mL) and extracted with EtOAc (3×50 mL). The combined organic layer was washed with NaHCO₃ (3×50 mL), dried over MgSO₄, filtered, and concentrated under reduced pressure. The residue was purified by column chromatography (SiO₂, Petroleum ether/Ethyl acetate=20/1 to 5/1). Compound WA004_5 (25 g, 65%) was obtained as a yellow solid.

To a solution of compound WA004_5 (15.0 g, 105 mmol) in dichloromethane (500 mL) was added m-CPBA (42.5 g, 209 mmol, 85% purity). The mixture was stirred at 60° C. for 6 h. After quenching by Na₂SO₃ (3 eq), the reaction mixture was diluted with water (50 mL) and extracted with EtOAc (3×50 mL). The combined organic layer was washed with NaHCO₃ (3×50 mL), dried over MgSO₄, filtered, and concentrated under reduced pressure. The residue was purified by column chromatography (SiO₂, dichloromethane/MeOH=20/1 to 10/1). Compound WA004_6 (15.0 g, 89.9%) was obtained as a brown solid.

A solution of compound WA004_6 (15.0 g, 94.2 mmol) in Ac₂O (30 mL) was stirred at 140° C. for 4 h. The reaction mixture was filtered and concentrated under reduced pressure to give the crude product, which was used in the next step without further purification. Compound WA004_7 (16.0 g, 84.4%) was obtained as a brown solid.

To a solution of compound WA004_7 acetate (16.0 g, 79.5 mmol) in MeOH (200 mL) was added NaOH (4.77 g, 119 mmol). The mixture was stirred at 20° C. for 6 h. The reaction mixture was filtered and concentrated under reduced pressure. The crude product was used in the next step without further purification. Compound WA004_8 (12.0 g, 94.8%) was obtained as a brown solid.

A solution of compound WA004_8 (12.0 g, 75.4 mmol) in POCl₃ (100 mL) was stirred at 100° C. for 4 h. The reaction mixture was quenched by the addition of Na₂HCO₃ (500 mL) at 0° C., and then diluted with water (100 mL) and extracted with EtOAc (2×100 mL). The combined organic layer was washed with water (2×100 mL), dried over MgSO₄, filtered, and concentrated under reduced pressure. The residue was purified by column chromatography (SiO₂, Petroleum ether/Ethyl acetate=20/1 to 5/1). Compound WA004 (8.00 g, 59.7%) was obtained as a brown solid.

¹H NMR (400 MHZ, CDCl₃) δ 8.13 (d, J=5.5 Hz, 1H), 7.61 (d, J=8.0 Hz, 1H), 7.53-7.43 (m, 2H), 7.41-7.22 (m, 1H), 3.00 (s, 3H).

### General Procedure for Preparation of WA005

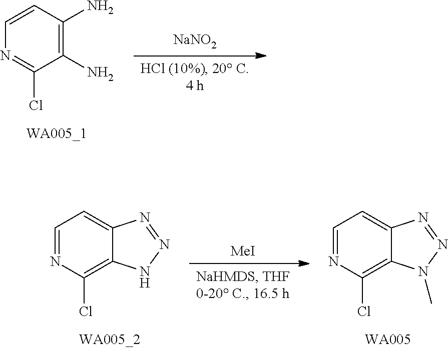

### Step 1:

To a solution of compound WA005_1 (45.0 g, 313 mmol) in 10% aq. HCl (1350 mL) was added NaNO₂ (43.2 g, 627 mmol). The mixture was stirred at 20° C. for 4 h. The reaction mixture was concentrated under reduced pressure at 45° C. The crude product was used in the next step without further purification. Compound WA005_2 (77 g, crude, HCl salt) was obtained as a white solid.

¹H NMR (400 MHZ, CDCl₃) δ ppm 8.30-8.34 (m, 1H), 7.90 (br d, J=5.50 Hz, 1H).

To a solution of compound WA005_2 (55.0 g, 356 mmol) in THF (800 mL) was added LiHMDS dropwise (1 M, 534 mL) at 0° C. The reaction mixture was stirred at 0° C. for 30 min. Then, Mel (303 g, 2.14 mol) was added dropwise at 20° C., and the mixture was stirred at 20° C. for 16 h under N₂ atmosphere. The reaction mixture was a yellow solution. The reaction mixture was quenched by pouring into H₂O (1000 mL) at 0° C., and the mixture was stirred for 1 h. The reaction solution was concentrated under reduced pressure to remove THF, and then filtered to give a residue. The H₂O layer was extracted with dichloromethane (3×3000 mL). The combined organic layer was dried over MgSO₄, filtered, and concentrated under reduced pressure. The residue was purified by flash silica gel chromatography (Eluent of 0˜50% Ethyl acetate/Petroleum ether). Compound WA005 (20.0 g, 33.3%) was obtained as a white solid.

¹H NMR (400 MHZ, DMSO-d₆) δ ppm 8.26-8.35 (m, 1H), 8.11 (d, J=5.75 Hz, 1H), 4.56 (s, 3H).

### General Procedure for Preparation of Compound WA006

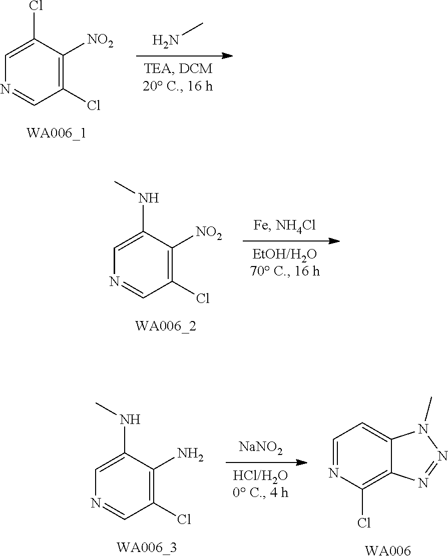

### Step 1

To a solution of compound WA006_1 (20.0 g, 104 mmol) in dichloromethane (300 mL) was added triethylamine (13.6 g, 135 mmol, 18.7 mL) and methanamine hydrochloride salt (9.80 g, 145.09 mmol). The mixture was stirred at 45° C. for 16 h. The reaction mixture was filtered and concentrated under reduced pressure. The residue was purified by column chromatography (SiO₂, Petroleum ether/Ethyl acetate=20/1 to 5/1). Compound WA006_2 (15.0 g, 77.2%) was obtained as a yellow solid.

### Step 2:

To a solution of compound WA006_2 (10.0 g, 53.3 mmol) in H₂O (100 mL) and THF (100 mL) was added Fe (14.9 g, 267 mmol) and NH₄Cl (14.3 g, 266 mmol). The mixture was stirred at 70° C. for 16 h. The reaction mixture was diluted with water (50 mL) and extracted with EtOAc (3×50 mL). The combined organic layer was washed with NaHCO₃ (3×50 mL), dried over MgSO₄, filtered, and concentrated under reduced pressure. The crude product was used in the next step without further purification. Compound WA006_3 (7.00 g, 83.3%) was obtained as a yellow solid.

### Step 3:

To a solution of compound WA006_3 (7.00 g, 44.4 mmol, 1.00 eq) in 10% aq. HCl (200 mL) was added NaNO₂ (4.60 g, 66.6 mmol). The mixture was stirred at 0° C. for 4 h. The reaction mixture was filtered and concentrated under reduced pressure. The crude product was used into the next step without further purification. Compound WA006 (6 g, 80%) was obtained as a yellow solid.

### General Procedure for Preparation of Compound WA007

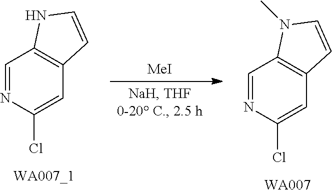

To a solution of compound WA007_1 (3.00 g, 19.7 mmol) in THF (30 mL) was added NaH (1.18 g, 29.5 mmol, 60% purity) at 0° C. under N₂. The mixture was stirred at 0° C. for 0.5 h. Then, Mel (3.35 g, 23.6 mmol) was added to the solution. The reaction was stirred at 20° C. for 2 h. The reaction mixture was quenched by the addition of saturated NH₄Cl (50 mL) at 25° C. and then diluted with H₂O (100 mL) and extracted with ethyl acetate (3×100 mL). The combined organic layer was washed with brine, dried over anhydrous sodium sulfate, filtered, and concentrated under reduced pressure. The residue was purified by flash silica gel chromatography (Eluent of 0˜40% Ethyl acetate/Petroleum ether). Compound WA007 (2.50 g, 76.3%) was obtained as a yellow solid.

¹H NMR (400 MHZ, DMSO-d₆) δ ppm 8.61 (s, 1H), 7.63 (s, 1H), 7.48 (d, J=3.25 Hz, 1H), 6.62 (dd, J=3.13, 0.63 Hz, 1H), 3.80 (s, 3H).

### General Procedure for Preparation of Compound WA008

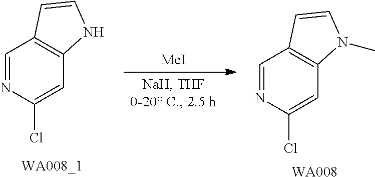

To a solution of compound WA008_1 (3.00 g, 19.7 mmol) in THF (30 mL) was added NaH (1.18 g, 29.5 mmol, 60% purity) at 0° C. under N₂. The mixture was stirred at 0° C. for 0.5 h. Then, Mel (3.35 g, 23.6 mmol) was added to the solution. The reaction was stirred at 20° C. for 2 h. The reaction mixture was quenched by the addition of saturated NH₄Cl (50 mL) at 25° C. and then diluted with H₂O (100 mL) and extracted with ethyl acetate (100 mL). The combined organic layer was washed with brine, dried over anhydrous sodium sulfate, filtered, and concentrated under reduced pressure. The residue was purified by flash silica gel chromatography (Eluent of 0˜40% Ethyl acetate/Petroleum ether). Compound WA008 (2.80 g, 68.4%, 80% purity) was obtained as a yellow solid.

¹H NMR (400 MHZ, DMSO-d₆) δ ppm 8.65 (s, 1H), 7.54-7.73 (m, 2H), 6.48 (d, J=2.88 Hz, 1H), 3.85-3.96 (m, 3H).

### General Procedure for Preparation of Compound WA012

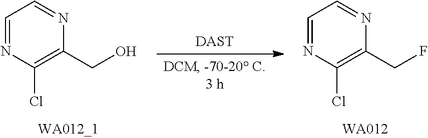

DAST (2.74 mL, 20.8 mmol) was added to dichloromethane (100 mL), cooled to −70° C., and treated dropwise with a solution of compound WA012_1 (2.50 g, 17.3 mmol) in dichloromethane (40 mL) under argon. The resulting mixture was allowed to warm to 20° C., and stirred for 3 h. The reaction was quenched by adding water (100 mL), and the solution was extracted with dichloromethane (2×100 mL). The combined organic layer was dried under magnesium sulfate and evaporated under reduced pressure. The residue was purified by flash silica gel chromatography (Eluent of 0˜30% Ethyl acetate/Petroleum ether). Compound WA012 (3.00 g, 50.9%) was obtained as a white solid.

¹H NMR (400 MHZ, CDCl₃) δ ppm 8.57 (br s, 1H), 8.37-8.44 (m, 1H), 5.51-5.74 (m, 2H).

### Synthesis of 14 Acid BBs

### Part I:

### General Procedure for Preparation of Compound WB006

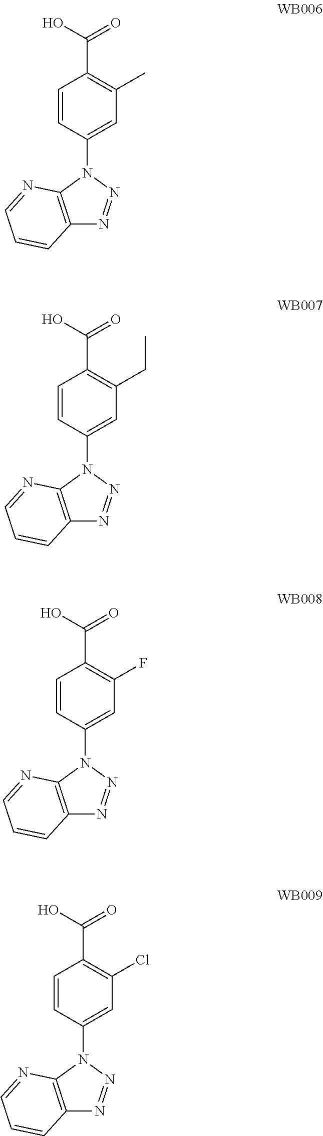 [SMILES: Cc1cc(-n2nnc3cccnc32)ccc1C(=O)O ; CCc1cc(-n2nnc3cccnc32)ccc1C(=O)O ; O=C(O)c1ccc(-n2nnc3cccnc32)cc1F ; O=C(O)c1ccc(-n2nnc3cccnc32)cc1Cl]

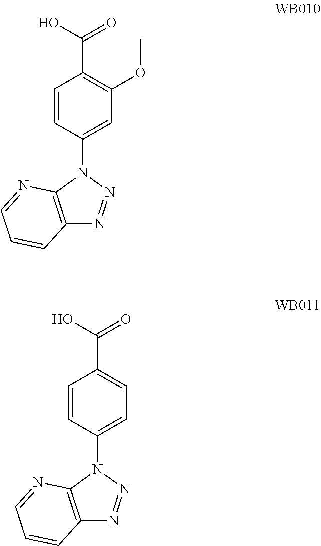 [SMILES: COc1cc(-n2nnc3cccnc32)ccc1C(=O)O ; O=C(O)c1ccc(-n2nnc3cccnc32)cc1]

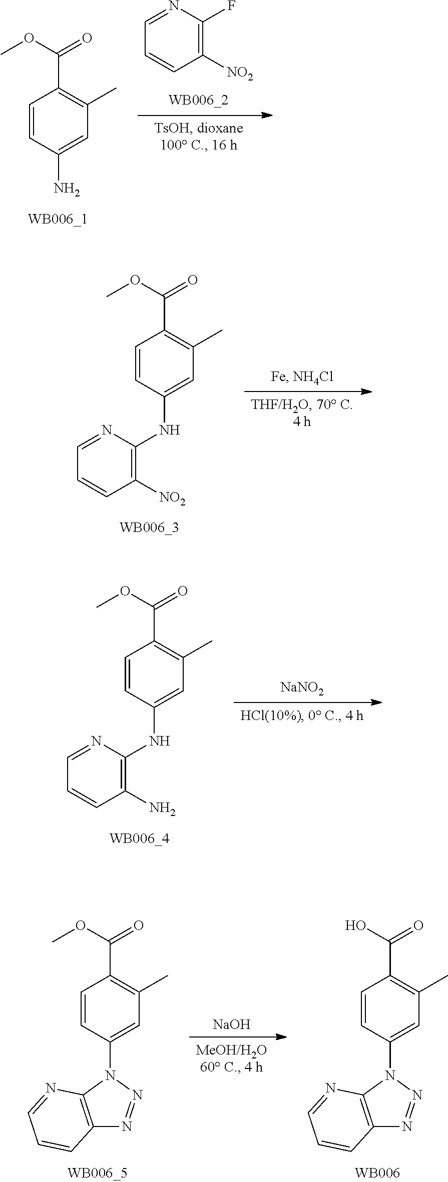

### Step 1:

To a solution of compound WB006_2 (23.0 g, 161 mmol) in dioxane (200 mL) was added TsOH (56.0 g, 294 mmol) and compound WB006_1 (24.3 g, 147 mmol). The mixture was stirred at 100° C. for 16 h. The reaction mixture was filtered and concentrated under reduced pressure. The crude product was used for next step directly without further purification. Compound WB006_3 (20.0 g, 47.3%) was obtained as a brown solid.

### Step 2:

To a solution of compound WB006_3 (20.0 g, 69.6 mmol) in H₂O (100 mL) and THF (100 mL) was added Fe (19.4 g, 348 mmol) and NH₄Cl (18.6 g, 348 mmol). The mixture was stirred at 70° C. for 4 h. The reaction mixture was filtered and concentrated under reduced pressure to give a filter liquor. The filter liquor was further concentrated under reduced pressure. The crude product was used into the next step without further purification. Compound WB006_4 (15.00 g, 83.7%) was obtained as a black solid.

### Step 3:

To a solution of compound WB006_4 (15.0 g, 58.3 mmol) in 10% aq. HCl (500 mL) was added NaNO₂ (6.03 g, 87.4 mmol). The mixture was stirred at 0° C. for 4 h. The reaction mixture was filtered and concentrated under reduced pressure. The crude product was used in the next step without further purification. Compound WB006_5 (9.00 g, 57.5%) was obtained as a brown solid.

### Step 4:

To a solution of compound WB006_5 (9.00 g, 33.6 mmol) in MeOH (100 mL) was added NaOH (2.68 g, 67.1 mmol). The mixture was stirred at 60° C. for 4 h. The reaction mixture was filtered and concentrated under reduced pressure. The residue was purified by column chromatography (SiO₂, Petroleum ether/Ethyl acetate=20/1 to 5/1). Compound WB006 (8.00 g, 93.8%) was obtained as a brown solid.

¹H NMR (400 MHZ, DMSO-d₆) δ 8.92 (dd, J=1.3, 4.4 Hz, 1H), 8.75 (dd, J=1.3, 8.4 Hz, 1H), 8.27 (brs, 1H), 8.24 (s, 1H), 8.13 (d, J=8.4 Hz, 1H), 7.66 (dd, J=4.6, 8.3 Hz, 1H), 2.82-2.64 (m, 3H).

### General Procedure for Preparation of Compound WB007

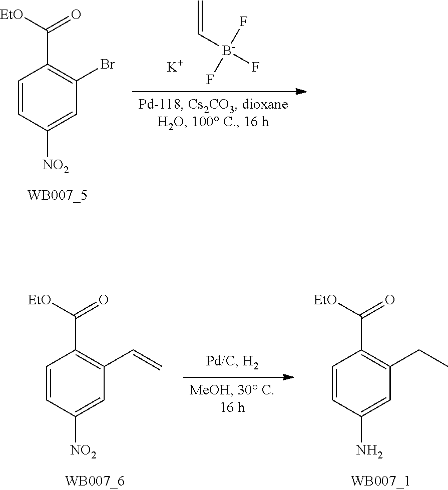

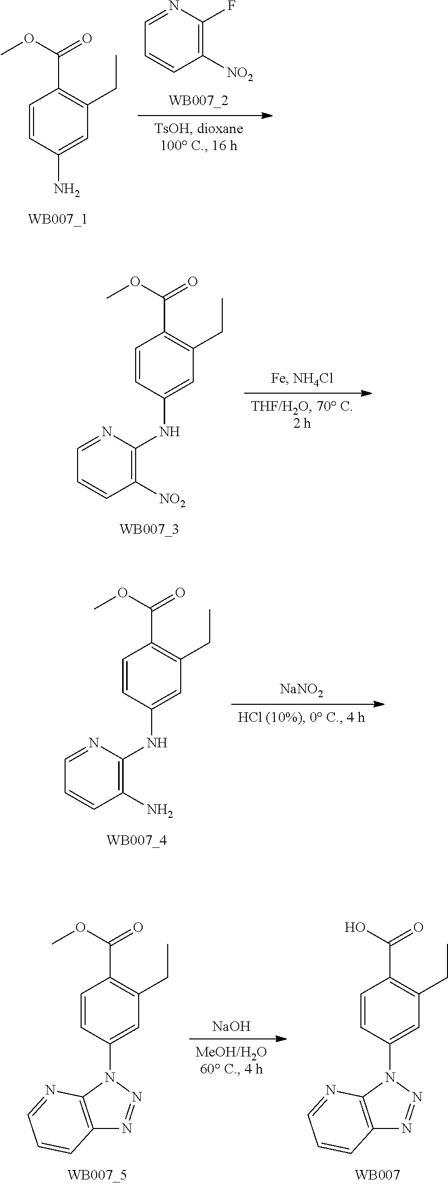

### Step 1:

To a solution of compound WB007_5 (25.0 g, 96.1 mmol) and potassium vinyltrifluoroborate (14.2 g, 105.8 mmol) in water (20.0 mL) and dioxane (100 mL) was added Cs₂CO₃ (62.7 g, 192 mmol) and Pd-118 (1.25 g, 1.92 mmol). The mixture was stirred at 100° C. for 16 h. The reaction mixture was filtered and concentrated under reduced pressure. The residue was purified by column chromatography (SiO₂, Petroleum ether/Ethyl acetate=20/1 to 5/1). Compound WB007_6 (18.0 g, 90.4%) was obtained as a brown solid.

### Step 2:

To a solution of WB007_6 (18.0 g, 86.9 mmol, 1.00 eq) in MeOH (150 mL) and EtOAc (150 mL) was added Pd/C (5.00 g, 10 wt %) under N₂. The suspension was degassed and purged with H₂ several times. The mixture was stirred under H₂ (50 psi) at 30° C. for 16 hours. The reaction mixture was filtered and concentrated under reduced pressure at 40° C. to afford crude product. The crude product was used in the next step directly without further purification. Compound WB007_1 (12.0 g, 77.1%) was obtained as a yellow solid.

### Steps 3-6:

WB007 was prepared according to the procedure described for WB006 by substituting methyl 4-amino-2-methylbenzoate with methyl 4-amino-2-ethylbenzoate, provided the desired product as a solid. ¹H NMR (400 MHZ, DMSO-d₆) δ 8.94 (dd, J=1.4, 4.5 Hz, 1H), 8.77 (dd, J=1.4, 8.4 Hz, 1H), 8.31 (d, J=2.1 Hz, 1H), 8.27 (d, J=8.8 Hz, 1H), 8.12 (d, J=8.4 Hz, 1H), 7.69 (dd, J=4.5, 8.4 Hz, 1H), 3.30-3.08 (m, 2H), 1.30 (t, J=7.4 Hz, 3H).

### General Procedure for Preparation of Compound WB008

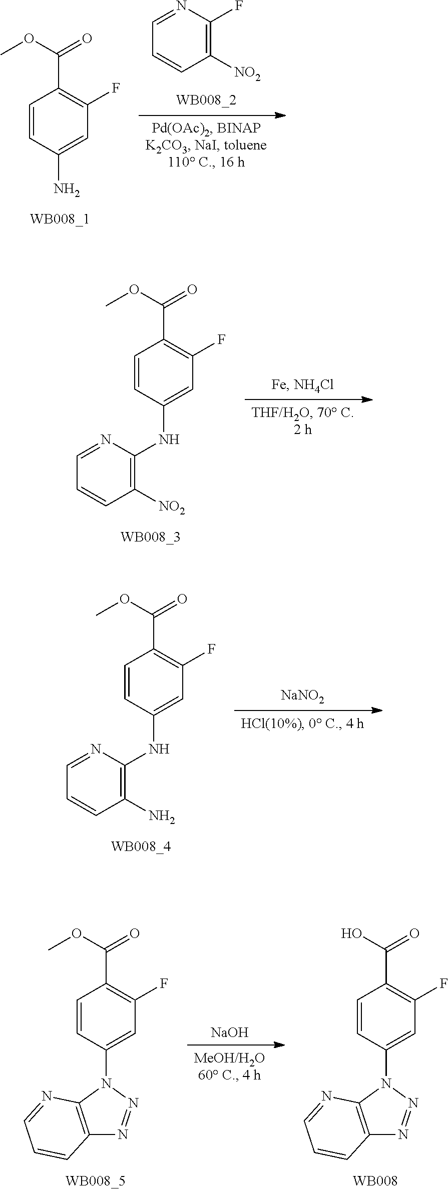

WB008 was prepared according to the procedure described for WB006 by substituting methyl 4-amino-2-methylbenzoate with methyl 4-amino-2-fluorobenzoate, provided the desired product as a solid.

%) ¹H NMR (400 MHZ, DMSO-d₆) δ ppm 12.16-14.11 (m, 1H), 8.91-9.01 (m, 1H), 8.76-8.81 (m, 1H), 8.31-8.47 (m, 2H), 8.16-8.27 (m, 1H), 7.64-7.76 (m, 1H).

### General Procedure for Preparation of Compound WB009

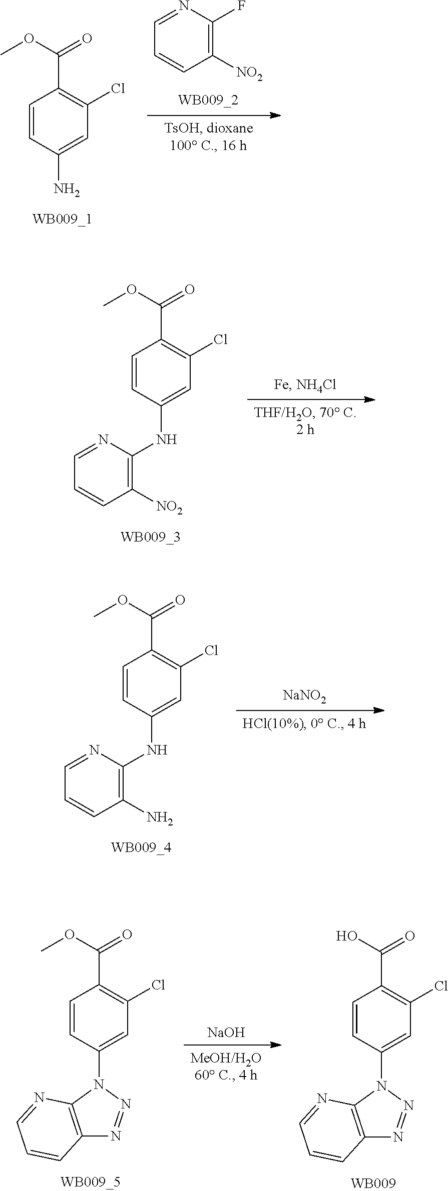

WB009 was prepared according to the procedure described for WB006 by substituting methyl 4-amino-2-methylbenzoate with methyl 4-amino-2-chlorobenzoate, provided the desired product as a solid.

¹H NMR (400 MHZ, DMSO-d₆) δ 8.93 (dd, J=1.3, 4.5 Hz, 1H), 8.77 (dd, J=1.1, 8.4 Hz, 1H), 8.58 (d, J=2.0 Hz, 1H), 8.41 (dd, J=1.9, 8.6 Hz, 1H), 8.14 (d, J=8.5 Hz, 1H), 7.68 (dd, J=4.5, 8.4 Hz, 1H).

### General Procedure for Preparation of Compound WB010

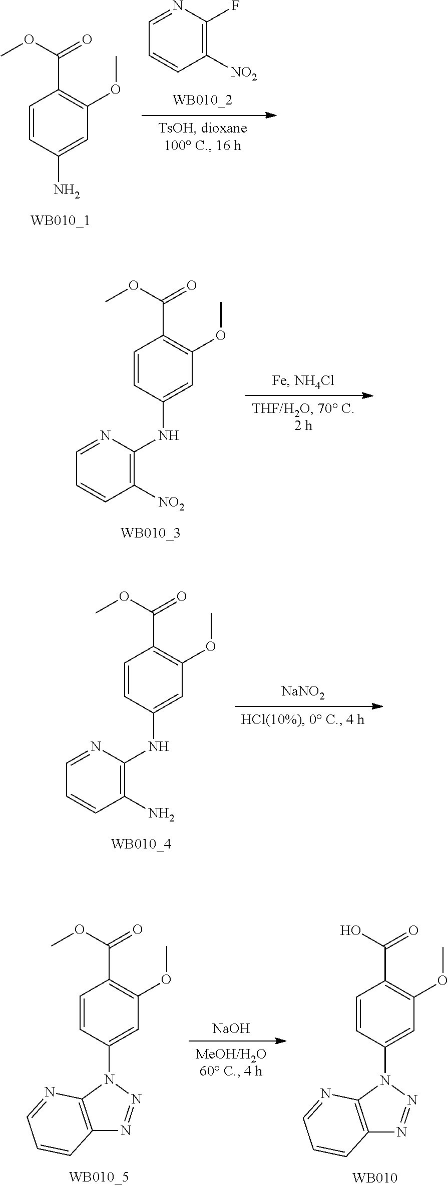

WB010 was prepared according to the procedure described for WB006 by substituting methyl 4-amino-2-methylbenzoate with methyl 4-amino-2-methoxybenzoate, provided the desired product as a solid.

¹H NMR (400 MHZ, DMSO-d₆) δ ppm 12.88 (br s, 1H) 1H), 8.88 (dd, J=4.50, 1.38 Hz, 1H) 1H), 8.72 (dd, J=8.32, 1.31 Hz, 1H) 1H), 8.05 (d, J=1.50 Hz, 1H) 1H), 7.92-8.00 (m, 2H), 7.63 (dd, J=8.38, 4.50 Hz, 1H) 1H), 3.96 (s, 3H).

### General Procedure for Preparation of Compound WB011

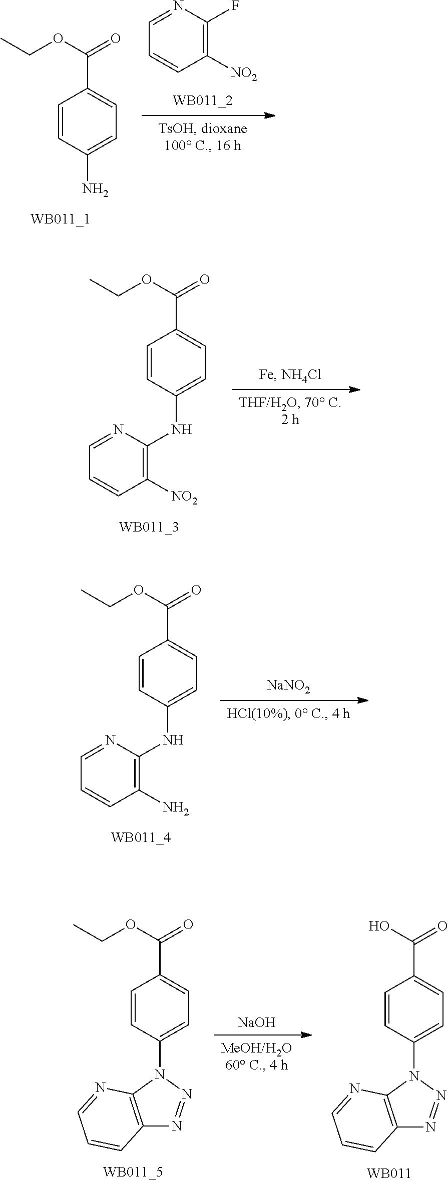

WB011 was prepared according to the procedure described for WB006 by substituting methyl 4-amino-2-methylbenzoate with methyl 4-aminobenzoate, provided the desired product as a solid.

¹H NMR (400 MHZ, DMSO-d₆) δ 13.24 (br s, 1H), 8.90 (dd, J=1.2, 4.4 Hz, 1H), 8.74 (dd, J=1.3, 8.4 Hz, 1H), 8.50-8.43 (m, J=8.6 Hz, 2H), 8.26-8.21 (m, J=8.8 Hz, 2H), 7.66 (dd, J=4.5, 8.4 Hz, 1H).

### Part II:

### General Procedure for Preparation of Compound WB013

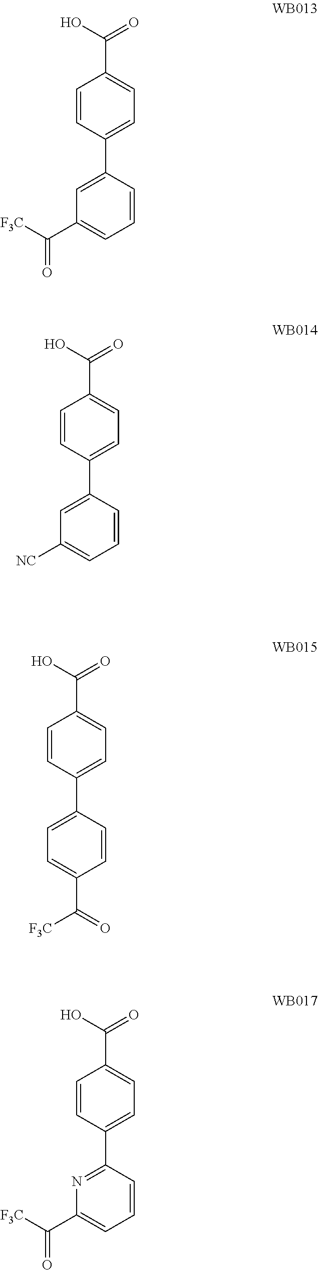 [SMILES: O=C(O)c1ccc(-c2cccc(C(=O)C(F)(F)F)c2)cc1 ; N#Cc1cccc(-c2ccc(C(=O)O)cc2)c1 ; O=C(O)c1ccc(-c2ccc(C(=O)C(F)(F)F)cc2)cc1 ; O=C(O)c1ccc(-c2cccc(C(=O)C(F)(F)F)n2)cc1]

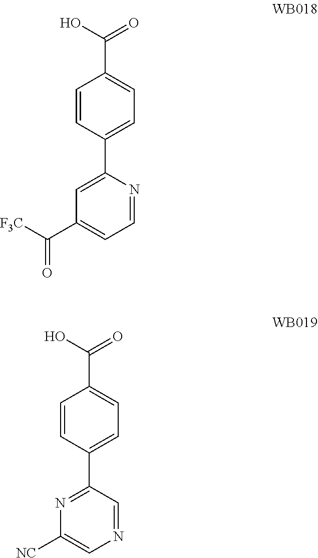 [SMILES: O=C(O)c1ccc(-c2cc(C(=O)C(F)(F)F)ccn2)cc1 ; N#Cc1cncc(-c2ccc(C(=O)O)cc2)n1]

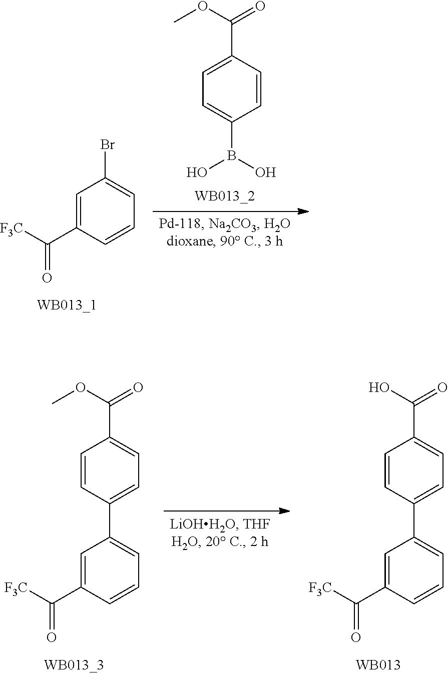

### Step 1:

To a solution of compound WB013_1 (5.00 g, 19.8 mmol), Na₂CO₃ (4.19 g, 39.5 mmol) and compound WB013_2 (3.91 g, 21.7 mmol) in H₂O (20 mL) and dioxane (100 mL) was added Pd-118 (644 mg, 988 μmol) under N₂. The reaction was stirred at 90° C. for 3 h. The mixture was concentrated under reduced pressure at 50° C. The residue was poured into water (100 mL), and the aqueous phase was extracted with ethyl acetate (3×100 mL). The combined organic phase was washed with brine (2×100 mL), dried with anhydrous MgSO₄, filtered, and concentrated in vacuum. The residue was purified by flash silica gel chromatography (ISCO®; 40 g SepaFlash® SilicaFlash Column, eluent of 0-10% EtOAc/PE) to afford desired product. WB013_3 (5.26 g, 86.4%) was obtained as a brown solid.

### Step 2:

To a solution of compound WB013_3 (5.26 g, 17.1 mmol) in THF (100 mL) and H₂O (50 mL) was added LiOH·H₂O (1.43 g, 34.1 mmol). The reaction was stirred at 20° C. for 2 h. The reaction mixture was concentrated under reduced pressure, and the residue was poured into water (100 mL) and stirred for 3 min. The aqueous phase was extracted with ethyl acetate (3×100 mL). The organic phase was discarded. The aqueous phase was adjusted to pH 4-5 by 1N aq. HCl. The precipitate was collected and dried. to afford Compound WB013 (2.09 g, 41.6%) was obtained as a white solid.

¹H NMR (400 MHZ, CDCl₃) δ ppm 8.35 (s, 1H), 8.27 (d, J=8.25 Hz, 2H), 8.11-8.16 (m, 1H), 7.98-8.03 (m, 1H), 7.70-7.78 (m, 3H).

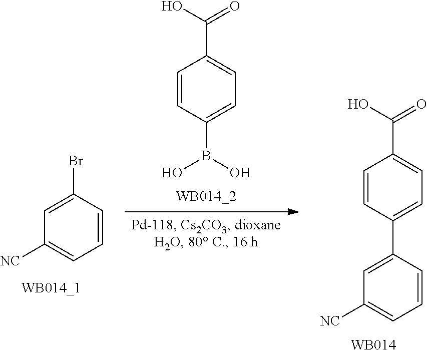

### General Procedure for Preparation of Compound WB014

To a solution of compound WB014-1 (5.00 g, 27.5 mmol), Cs₂CO₃ (17.9 g, 54.9 mmol), and compound WB014-2 (5.01 g, 30.2 mmol) in dioxane (100 mL) and H₂O (20 mL) was added Pd-118 (895 mg, 1.37 mmol) under N₂. The reaction was stirred at 80° C. for 16 h. The reaction mixture was concentrated under reduced pressure. The residue was purified by flash silica gel chromatography (Eluent of 0˜10% THF/MEOH). Compound WB014 (4.00 g, 65.2%) was obtained as a brown solid.

¹H NMR (400 MHZ, DMSO-d₆) δ ppm 8.17-8.20 (m, 1H), 8.03-8.08 (m, 1H), 7.99 (d, J=8.25 Hz, 2H), 7.81-7.87 (m, 1H), 7.74 (d, J=8.38 Hz, 2H), 7.65-7.70 (m, 1H).

### General Procedure for Preparation of Compound WB015

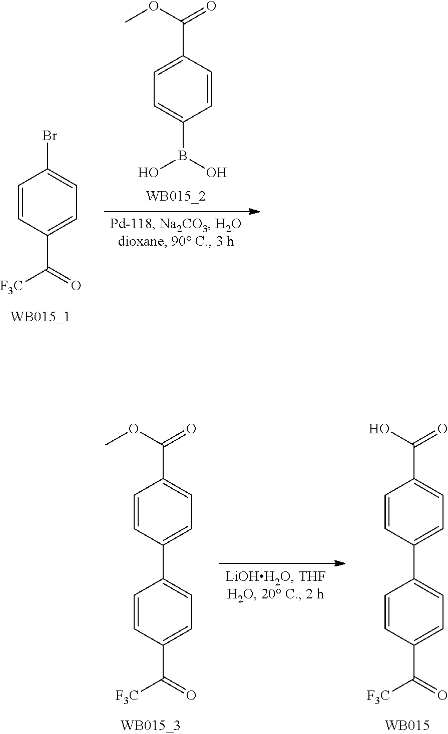

### Step 1:

A mixture of compound WB015_1 (4.50 g, 17.8 mmol), compound WB015_2 (3.84 g, 21.3 mmol), Na₂CO₃ (3.77 g, 35.6 mmol) in dioxane (100 mL), and H₂O (25 mL) was degassed and purged with N₂ thrice, and then Pd-118 (1.03 g, 889 μmol) was added to the mixture. The mixture was stirred at 90° C. for 3 h under N₂ atmosphere. The reaction mixture was filtered and concentrated under reduced pressure. The residue was purified by flash silica gel chromatography (Eluent of 0˜30% Ethyl acetate/Petroleum ether, gradient). Compound WB015_3 (4.50 g, 82%) was obtained as a yellow solid.

¹H NMR (400 MHZ, CDCl₃) δ ppm 8.04-8.14 (m, 4H), 7.68-7.78 (m, 2H), 7.59-7.67 (m, 2H), 3.88 (s, 3H).

### Step 2:

To a solution of compound WB015_3 (4.50 g, 14.6 mmol) in THF (45 mL) and H₂O (15 mL) was added LiOH·H₂O (1.23 g, 29.2 mmol). The mixture was stirred at 20° C. for 3 h. The reaction mixture was concentrated under reduced pressure. After adjusting to pH 3 with HCl (2 M, 20 mL). The precipitate was collected and dried to afford Compound WB015 (2.4 g, 55.88%) as a white solid.

¹H NMR (400 MHZ, CDCl₃) δ ppm 8.23 (dd, J=13.20, 8.19 Hz, 4H), 7.64-7.93 (m, 4H).

### General Procedure for Preparation of Compound WB017

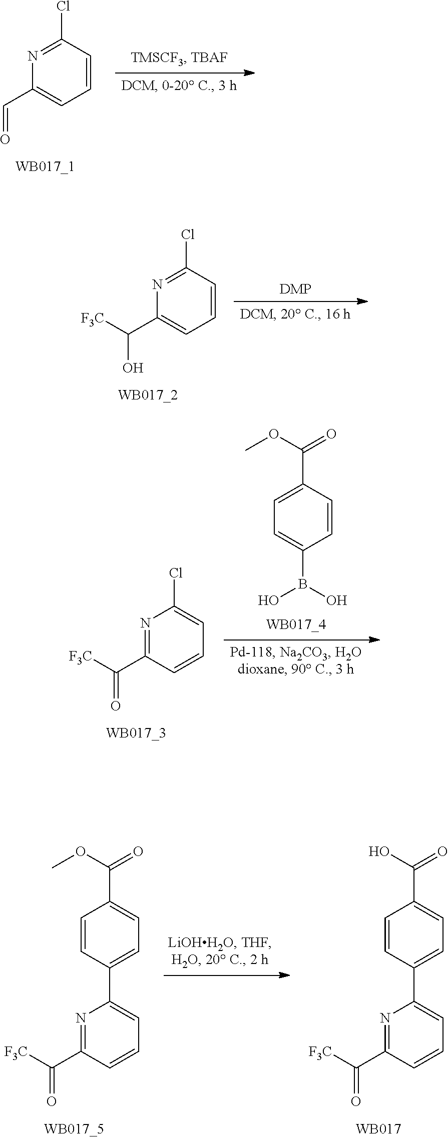

### Step 1:

To a solution of compound WB017_1 (10.0 g, 70.6 mmol) in dichloromethane (100 mL) was chilled to 0° C. in an ice/water bath was added dropwise TMSCF₃ (12.1 g, 84.8 mmol, 1.20 eq) and followed by the dropwise addition of TBAF (1 M, 1.06 mL). The reaction was allowed to warm to 20° C., stirred for 2 h, and then additional TBAF (1 M, 35.3 mL) was slowly added. The reaction mixture was stirred at 20° C. for 1 h. The reaction was diluted with EtOAc (500 mL), washed with 0.1N aq. HCl (200 mL) and brine, dried over Na₂SO₄, filtered, and concentrated under reduced pressure. The crude product was used in the next step directly without further purification. Compound WB017_2 (15.0 g, crude) was obtained as a brown solid.

¹H NMR (400 MHZ, CDCl₃) δ ppm 7.69 (t, J=7.82 Hz, 1H), 7.32 (dd, J=15.01, 7.75 Hz, 2H), 4.89-5.06 (m, 1H), 4.69 (br d, J=9.38 Hz, 1H).

### Step 2:

To a solution of compound WB017_2 (15.0 g, 70.9 mmol) in dichloromethane (150 mL) was added Dess-Martin periodinane (36.1 g, 85.1 mmol). The reaction was stirred at 20° C. for 16 h. The reaction was then diluted with dichloromethane (250 mL), washed with saturated NaHCO₃ solution (2×100 mL) and brine (1×100 mL), dried over Na₂SO₄, filtered, and concentrated under reduced pressure. The residue was purified by flash silica gel chromatography (Eluent of 0˜50% Ethyl acetate/Petroleum ether). Compound WB017_3 (6.00 g, 40.4%) was obtained as a yellow oil.

¹H NMR (400 MHZ, DMSO-d₆) δ ppm 7.92-8.09 (m, 1H), 7.76 (s, 1H), 7.59 (d, J=7.88 Hz, 1H).

### Step 3:

To a solution of compound WB017-3 (5.63 g, 26.8 mmol), Na₂CO₃ (5.69 g, 53.7 mmol) and compound WB017-4 (5.31 g, 29.5 mmol) in H₂O (20 mL) and dioxane (100 mL) was added Pd-118 (875 mg, 1.34 mmol) under N₂. The reaction was stirred at 90° C. for 3 h. The reaction mixture was concentrated under reduced pressure, and the residue was poured into water (100 mL). The aqueous phase was extracted with ethyl acetate (3×100 mL). The combined organic phase was washed with brine (100 mL), dried with anhydrous MgSO₄, filtered, and concentrated in vacuum. The residue was purified by flash silica gel chromatography (Eluent of 0˜35% Ethyl acetate/THF) to afford desired product. Compound WB017-5 (5.74 g, 69%) was obtained as a brown solid.

### Step 4:

To a solution of compound WB017-5 (2.87 g, 9.28 mol, 1. THF (50 mL) and H₂O (25 mL) was added LiOH·H₂O (779 mg, 18.6 mmol). The reaction was stirred at 20° C. for 2 h. The reaction mixture was concentrated under reduced pressure, and then the residue was poured into water (100 mL) and stirred for 3 min. The aqueous phase was extracted with ethyl acetate (3×100 mL). The organic phase was discarded. The aqueous phase was adjusted to pH 4-5 with 1N aq. HCl. The precipitate was collected and dried to afford Compound WB017 (2.22 g, 81.03%) as a white solid.

¹H NMR (400 MHZ, CDCl₃) δ ppm 8.15-8.22 (m, 3H), 7.93-8.11 (m, 3H), 7.84-7.90 (m, 1H), 7.76 (d, J=7.75 Hz, 1H).

### General Procedure for Preparation of Compound WB018

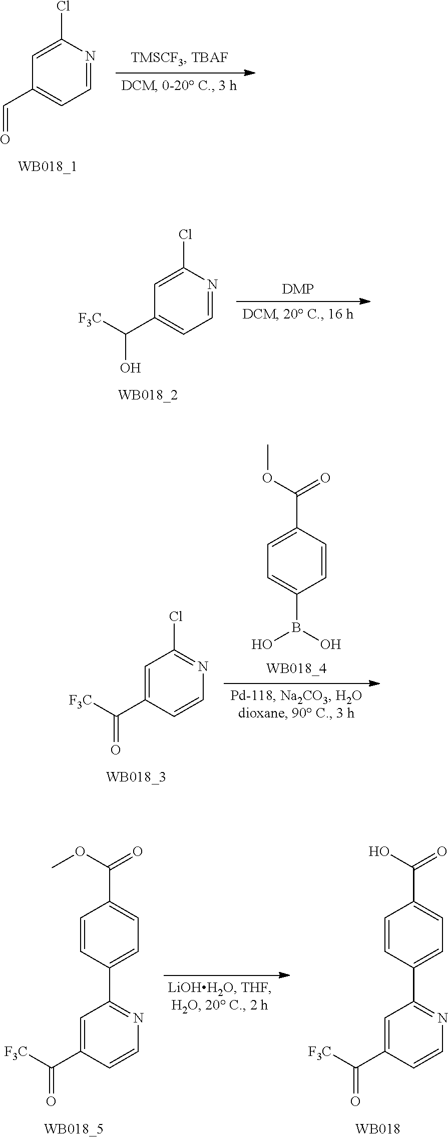

Compound WB018_3 was synthesized according to the procedure described for Compound WB017 by substituting 6-chloropicolinaldehyde with 2-chloroisonicotinaldehyde, provided the title compound as a white solid.

¹H NMR (400 MHZ, DMSO-d₆) δ ppm 8.80 (d, J=5.00 Hz, 1H), 8.17-8.21 (m, 2H), 8.10 (t, J=8.51 Hz, 3H), 8.00 (br s, 2H), 7.61 (d, J=5.00 Hz, 1H).

### General Procedure for Preparation of Compound WB019

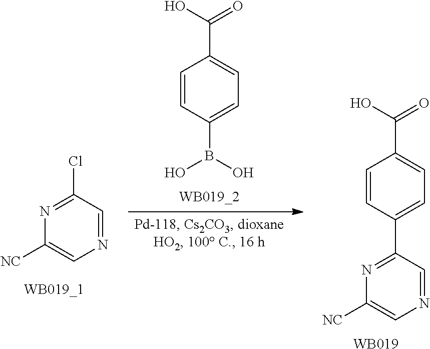

Compound WB015 was synthesized according to the procedure described for Compound WB017 by substituting 1-(4-bromophenyl)-2,2,2-trifluoroethan-1-one with 6-chloropyrazine-2-carbonitrile, provided the title compound as a white solid.

¹H NMR (400 MHZ, DMSO-d₆) δ ppm 12.54 (br s, 1H), 8.62 (s, 1H), 7.77-7.90 (m, 2H), 7.60 (d, J=8.76 Hz, 2H), 2.95 (s, 6H).

### General Procedure for Preparation of Compound WB035

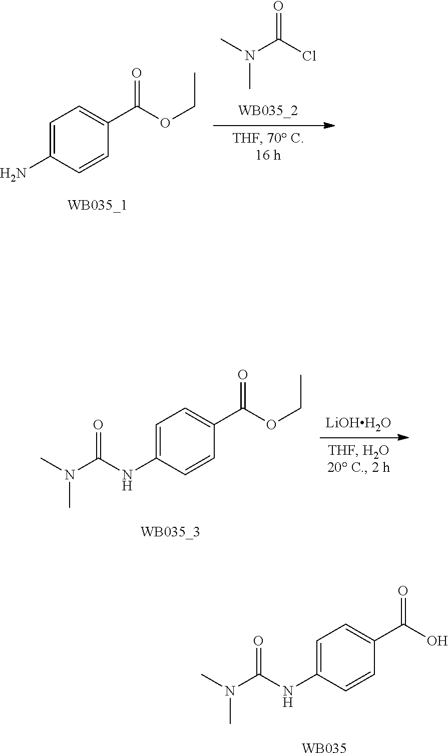

### Step 1:

To a solution of compound WB035_1 (20.0 g, 121 mmol) in THF (200 mL) was added compound WB035_2 (39.1 g, 363 mmol). The mixture was stirred at 70° C. for 16 h. The reaction mixture was concentrated under reduced pressure, and the residue was diluted with H₂O (100 mL) and extracted with EtOAc (3×100 mL). The combined organic layer was dried over MgSO₄, filtered, and concentrated under reduced pressure to give a residue that was purified by flash silica gel chromatography (Eluent of 0˜25% THF/Petroleum ether Compound WB035_3 (3.60 g, 12.6%) was obtained as a white solid.

¹H NMR (400 MHZ, DMSO-d₆) δ ppm 8.57-8.73 (m, 1H), 7.83 (d, J=8.88 Hz, 2H) 7.60-7.66 (m, 2H) 4.27 (q, J=7.17 Hz, 2H) 2.86-3.03 (m, 6H) 1.27-1.35 (m, 3H).

### Step 2:

To a solution of compound WB035_3 (3.60 g, 15.2 mmol) in THF (50 mL) and H₂O (50 mL) was added LiOH H₂O (1.28 g, 30.5 mmol). The reaction mixture was stirred at 20° C. for 16 h. The reaction was concentrated under reduced pressure. The mixture was poured into water (50 mL), and the solution was adjusted to pH 4-5 by 1N aq. HCl. The precipitate was collected and dried to afford Compound WB035 (2.00 g, 63.0%) as a white solid.

¹H NMR (400 MHZ, DMSO-d₆) δ ppm 12.54 (br s, 1H), 8.62 (s, 1H), 7.77-7.90 (m, 2H), 7.60 (d, J=8.76 Hz, 2H), 2.95 (s, 6H).

### 1.2 Experimental Details for Final Compound Synthesis Using the Procedures in 1.1

### Procedure W1

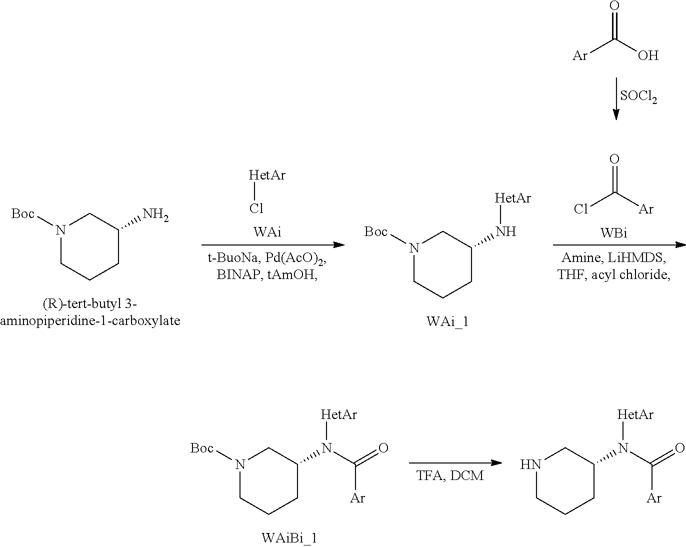

### Step 1

t-BuONa (30 mmol, 3.0 eq) was added to a solution of WAi (10 mmol, 1.0 eq) and (R)-tert-butyl 3-aminopiperidine-1-carboxylate (15 mmol, 3 g, 1.5 eq) in t-AmOH (60 mL) was added. Then, BINAP (0.5 mmol, 0.05 eq) and Pd(OAc)₂ (0.5 mmol, 0.05 eq) were added to the above reaction mixture under N₂. The reaction mixture was stirred at 110° C. for 16 h. The reaction mixture was concentrated under reduced pressure to remove solvent. The residue was diluted with H₂O (1.0 mL) and extracted with dichloromethane (3×1.5 mL). The combined organic layer was concentrated under reduced pressure to give WAi_1, which was purified by preparative TLC.

### Step 2

To a solution of WBi (100 μmol, 2.0 eq) in SOCl₂ (500 μL) was added two drops of DMF. The reaction mixture was shaken at 60° C. for 16 h and then concentrated under reduced pressure to give WBi_1, which was used without further purification. LiHMDS (150 μmol, 1.0 M in THF, 3.0 eq) or NaH (150 μmol, 3.0 eq) was added to a solution of WAi_1 (50 μmol, 1.0 eq) in THF (200 μL) and stirred at 0° C. for 15 mins. A solution of crude acid chloride WBi_1 in THF (0.3 mL) was added to the above solution, and the reaction was stirred at 30° C. for 16 h. The reaction was diluted with H₂O (1.0 mL) and extracted with dichloromethane (3×1.5 mL). The combined organic layer was concentrated under reduced pressure to give WAiBi_1, which was used in the next step directly without further purification.

### Step 3

WAiBi_1 (50 μmol, 1.0 eq) was dissolved in a solution (1.0 mL) of dichloromethane/TFA (v/v=5/1), and the reaction was shaken at 30° C. for 2 h. The reaction mixture was then concentrated under reduced pressure to give a residue, which was purified by preparative HPLC to obtain the final product.

### Procedure W2

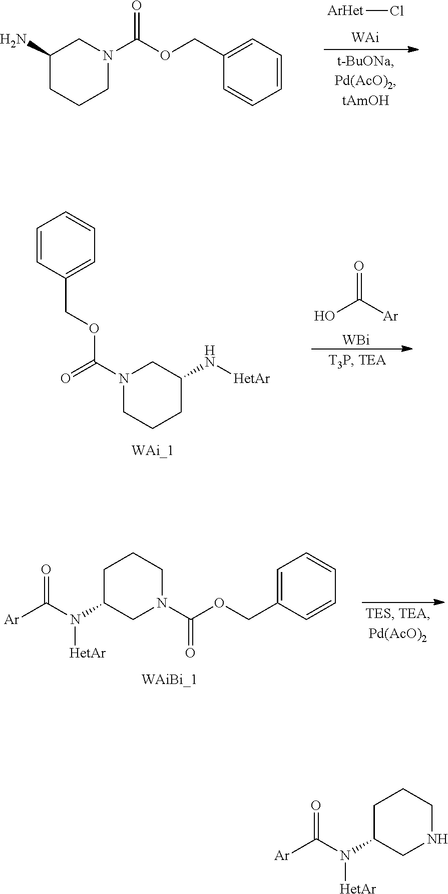

### Step 1

t-BuONa (30 mmol, 3.0 eq) was added to a solution of WAi (10 mmol, 1.0 eq) and (R)-benzyl 3-aminopiperidine-1-carboxylate in t-AmOH (60 mL). Then BINAP (0.5 mmol, 0.05 eq) and Pd(AcO)₂ (0.5 mmol, 0.05 eq) were added to the above reaction mixture under N₂. The reaction mixture was stirred at 110° C. for 16 h. The reaction mixture was concentrated under reduced pressure to remove solvent. The residue was diluted with H₂O (1.0 mL) and extracted with dichloromethane (3×1.5 mL). The combined organic layer was concentrated under reduced pressure to give WAi_1, which was purified by preparative TLC.

### Step 2

To a solution of WBi (100 μmol, 2.0 eq) and WAi_1 (50 μmol, 1.0 eq) in dioxane (500 μL) was added T₃P (150 μL. 50% wt in ethyl acetate) and triethylamine (150 μmol, 3.0 eq) under N₂. The reaction mixture was shaken at 110° C. for 16 h, after which the reaction was diluted with H₂O (1.0 mL) and extracted with dichloromethane (3×1.5 mL). The combined organic layer was concentrated under reduced pressure to give WAiBi_1, which was used in the next step directly.

### Step 3

WAiBi_1 (50 μmol, 1.0 eq) was dissolved in a solution of dichloromethane and MeOH (1.6 mL, v:v=1:1). Triethylsilane (200 μL) and triethylamine (500 μmol, 10.0 eq) were then added to the above solution. Finally, Pd(AcO)₂ (5 μmol, 1.0 eq) was added to the reaction mixture. The reaction mixture was stirred at 30° C. for 2 h, after which it was concentrated under reduced pressure. The residue was purified by preparative HPLC to obtain the final product.

### Procedure W3

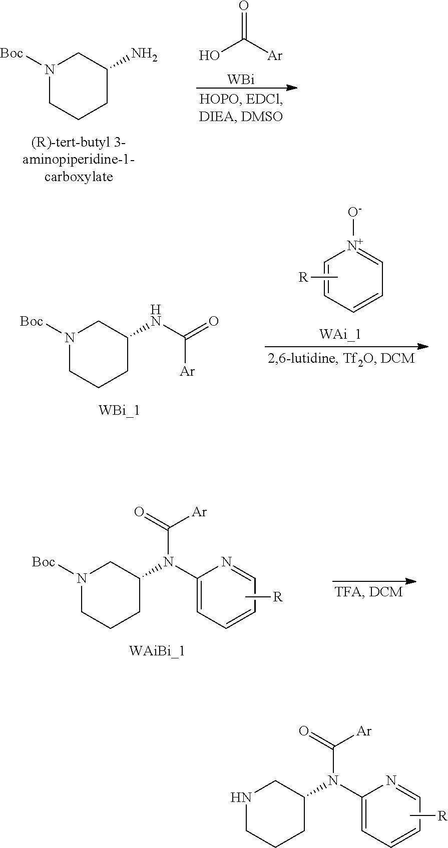

### Step 1

To a solution of WBi (150 μmol, 1.0 eq) and (R)-tert-butyl 3-aminopiperidine-1-carboxylate (100 μmol, 1.0 eq) in DMSO (0.5 mL) was added a solution of HOPO (120 μmol, 1.2 eq) and EDCI (120 μmol, 1.2 eq) in DMSO (0.5 mL). The reaction mixture was shaken at 50° C. for 16 h, after which the reaction mixture was concentrated under reduced pressure. The residue WAiBi_1 was purified by preparative TLC.

### Step 2

The following series of reactions were completed in a glove box. To a solution of WAiBi_1 (50 μmol, 1.0 eq) in dichloromethane (500 μL) was added 2, 6-lutidine (110 μmol, 2.2 eq). The reaction mixture was shaken at −30° C. for 0.5 h. Then, Tf₂O (55 μmol, 1.1 eq) was added to the reaction mixture, and the reaction mixture was shaken at −20° C. for 1 h. Next, a solution of Ai_1 (60 μmol, 1.2 eq) was added to the reaction, and the reaction mixture was shaken at 30° C. for 16 h. After 16 h, the reaction was concentrated under reduced pressure. The residue was diluted with H₂O (1.0 mL) and extracted with dichloromethane (3×1.5 mL). The combined organic layer was concentrated under reduced pressure to give WAiBi_1, which was used in the next step directly.

### Step 3

WAiBi_1 (50 μmol, 1.0 eq) was dissolved in a solution (1.0 mL) of dichloromethane/TFA (v/v=5/1). The reaction mixture was shaken at 30° C. for 2 h, after which it was concentrated under reduced pressure. The residue was purified by preparative HPLC to obtain the final product.

### Procedure W4

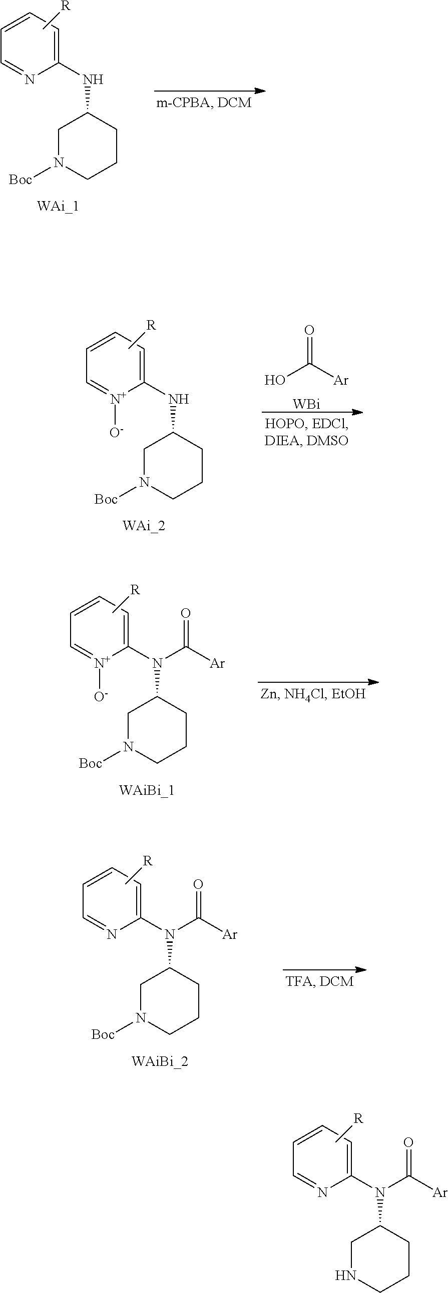

### Step 1

To a solution of WAi_1 (200 μmol, 1.0 eq) in dichloromethane (1 mL) was added a solution of m-CPBA (300 μmol, 1.5 eq) in dichloromethane (1 mL). The reaction mixture was shaken at 30° C. for 3 h, after which it was concentrated under reduced pressure to remove solvent. The residue was quenched with a saturated solution of NaHSO₃ and extracted with dichloromethane (1.5 mL). The combined organic layer was concentrated under reduced pressure to give WAi_2, which was purified by preparative TLC.

### Step 2

To a solution WAi_2 (100 μmol, 1.0 eq) and Bi (110 μmol, 1.1 eq) in dichloromethane (0.5 mL) was added DIEA (300 μmol, 3.0 eq). Then, a solution of HOPO (110 μmol, 1.1 eq) and EDCI (110 μmol, 1.1 eq) in dichloromethane (0.5 mL) was added to the reaction mixture. The reaction mixture was shaken at 50° C. for 16 h, after which the reaction was concentrated under reduced pressure. The residue was diluted with H₂O (1.0 mL) and extracted with dichloromethane (3×1.5 mL). The combined organic layer was concentrated under reduced pressure to give WAiBi_1, which was used in the next step directly.

### Step 3

To a solution of WAiBi_1 (50 μmol, 1.0 eq) in EtOH (0.8 mL) was added NH₄Cl (500 μmol, 10.0 eq) and activated Zn (300 μmol, 6.0 eq). The reaction mixture was shaken at 80° C. for 2 h, after which it was filtered and concentrated under reduced pressure. The residue was used in the next step directly.

### Step 4

WAiBi_2 (50 μmol, 1.0 eq) was dissolved in a solution (1.0 mL) of dichloromethane and TFA (v:v=5:1). The reaction mixture was shaken at 30° C. for 2 h. The reaction mixture was then concentrated under reduced pressure to give a residue, which was purified by preparative HPLC to obtain the final product.

### 2.1 Experimental Information for Intermediate Synthesis

### Experimental for Intermediate (WA001_1)

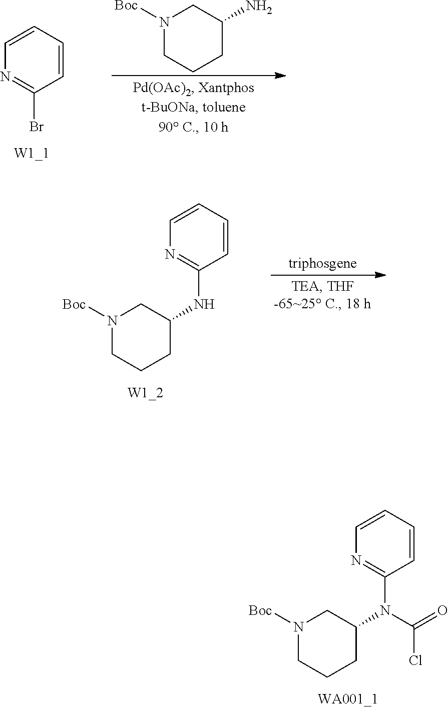 [SMILES: Brc1ccccn1 ; CC(C)(C)OC(=O)N1CCC[C@@H](N)C1 ; CC(C)(C)OC(=O)N1CCC[C@@H](Nc2ccccn2)C1 ; CC(C)(C)OC(=O)N1CCC[C@@H](N(C(=O)Cl)c2ccccn2)C1]

### Step 1:

Compound W1_1 (3.94 g, 24.97 mmol), tert-butyl (3R)-3-aminopiperidine-1-carboxylate (5 g, 24.97 mmol, specific optical rotation=−35° at 20° C. (concentration=5 in MeOH)), t-BuONa (4.80 g, 49.93 mmol), Xantphos (0.7223 g, 1.25 mmol), and Pd(OAc)₂ (0.2825 g, 1.25 mmol) were dissolved in toluene (300 mL), purged with N₂ for 2 minutes, and then heated at 90° C. for 10 h under N₂. The reaction mixture was poured into H₂O (100 mL) and extracted with ethyl acetate (3×100 mL). The combined organic layer was washed with brine (100 mL), dried over anhydrous Na₂SO₄, and concentrated. The residue was purified by flash silica gel chromatography (Eluent of 30% Ethyl acetate/Petroleum ether) to give compound W1_2 (3.2 g, 46%) as a white solid.

### Step 2:

A solution of compound W1_2 (1.5 g, 5.41 mmol) in THF (30 mL) was cooled to −65° C., and then triethylamine (3.28 g, 32.45 mmol) and triphosgene (4.95 g, 16.68 mmol) were added to the cooled solution. The mixture was allowed to slowly warm to 25° C., and then stirred at 25° C. for 18 h. The mixture was then filtered. The filtrate was quenched with saturated aq. NH₄Cl (30 mL) and extracted with ethyl acetate (3×30 mL). The combined organic layer was dried over MgSO₄, filtered, and concentrated. The residue was purified by flash silica gel chromatography (Eluent of 20% Ethyl acetate/Petroleum ether) to give WA001_1 (1.27 g, 57%) as a colorless oil.

¹H NMR (400 MHZ, CD₃OD) δ ppm 8.58 (dd, J=1.3, 4.9 Hz, 1H), 8.00 (dt, J=1.9, 7.7 Hz, 1H), 7.57-7.50 (m, 1H), 7.46 (d, J=7.9 Hz, 1H), 4.39 (td, J=2.1, 12.7 Hz, 1H), 4.20 (tt, J=4.0, 11.4 Hz, 1H), 3.95 (br d, J=12.1 Hz, 1H), 2.93 (br s, 1H), 2.55 (br s, 1H), 2.07 (br s, 1H), 1.74-1.64 (m, 1H), 1.59-1.41 (m, 11H).

### Experimental for Intermediate (WA002_1)

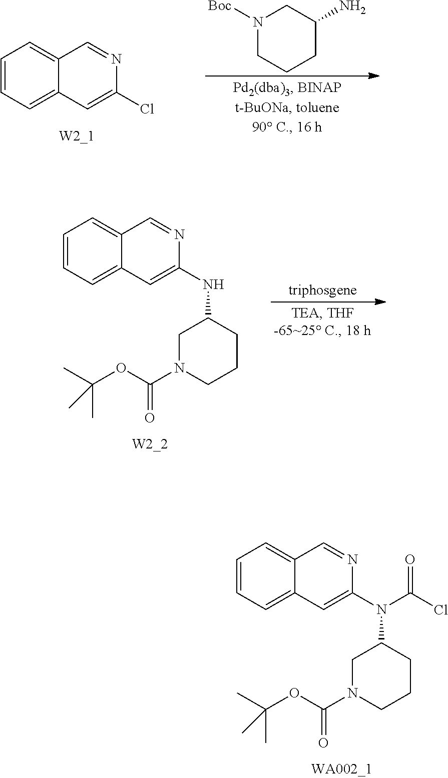

### Step 1:

Compound W2_1 (2.45 g, 14.98 mmol), tert-butyl (3R)-3-aminopiperidine-1-carboxylate (3.0 g, 14.98 mmol, specific optical rotation=−35° at 20° C. (concentration=5 in MeOH)), t-BuONa (2.88 g, 29.96 mmol), BINAP (1.37 g, 1.50 mmol) and Pd₂(dba)₃ (1.87 g, 3.00 mmol) were dissolved in toluene (50 mL), purged with N₂ for 2 min, and then heated at 90° C. for 16 h under N₂. The reaction mixture was poured into H₂O (50 mL) and extracted with ethyl acetate (3×50 mL). The organic phases were combined, washed with brine (50 mL), dried over anhydrous Na₂SO₄, and concentrated in vacuo to give a residue. The residue was purified by flash silica gel chromatography (Eluent of 30% Ethyl acetate/Petroleum ether) to give compound W1_2 (3.0 g, 61%) as a yellow solid.

¹H NMR (400 MHZ, CD₃OD) δ ppm 8.81 (s, 1H), 7.81 (d, J=8.3 Hz, 1H), 7.63-7.55 (m, 1H), 7.54-7.48 (m, 1H), 7.23 (t, J=7.5 Hz, 1H), 6.74 (s, 1H), 3.71 (s, 1H), 3.36-3.29 (m, 4H), 2.10 (s, 1H), 1.83 (s, 1H), 1.62 (dt, J=4.4, 8.8 Hz, 2H), 1.54-1.29 (m, 9H).

### Step 2:

A solution of compound W1_2 (1.5 g, 4.58 mmol) in THF (30 mL) was cooled to −65° C., and then triethylamine (2.78 g, 27.49 mmol,) and triphosgene (4.08 g, 13.74 mmol) were added to the cooled solution. The mixture was slowly allowed to warm to 25° C., and then stirred at 25° C. for 18 h. The mixture was then filtered. The filtrate was quenched with saturated aq. NH₄Cl (30 mL) and extracted with ethyl acetate (3×50 mL). The combined organic layer was dried over MgSO₄, filtered, and concentrated. The residue was purified by flash silica gel chromatography (Eluent of 30% Ethyl acetate/Petroleum ether) to give WA002_1 (1.02 g, 57%) as a white solid.

¹H NMR (400 MHZ, CD₃OD) δ ppm 9.28 (s, 1H), 8.22 (d, J=8.3 Hz, 1H), 8.06 (d, J=8.2 Hz, 1H), 7.91 (s, 1H), 7.90-7.86 (m, 1H), 7.84-7.78 (m, 1H), 4.46 (td, J=2.0, 12.7 Hz, 1H), 4.29 (tt, J=4.0, 11.2 Hz, 1H), 3.93 (br d, J=13.2 Hz, 1H), 2.97 (br s, 1H), 2.54 (br s, 1H), 2.13 (d, J=10.6 Hz, 1H), 1.75-1.65 (m, 1H), 1.60-1.40 (m, 11H).

### Experimental for Intermediate (WA003_1)

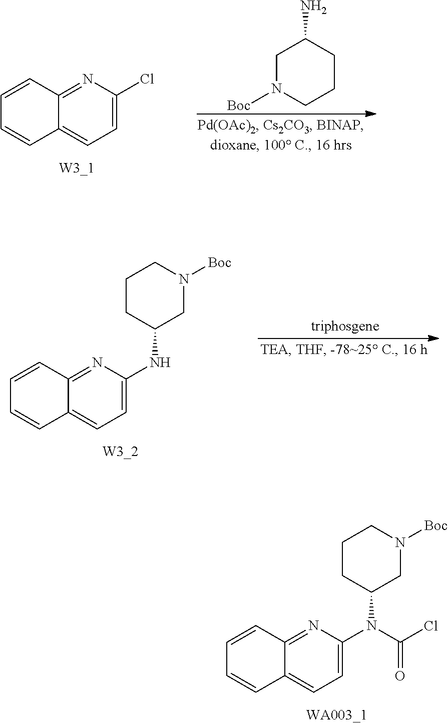 [SMILES: Clc1ccc2ccccc2n1 ; CC(C)(C)OC(=O)N1CCC[C@@H](N)C1 ; CC(C)(C)OC(=O)N1CCC[C@@H](Nc2ccc3ccccc3n2)C1 ; CC(C)(C)OC(=O)N1CCC[C@@H](N(C(=O)Cl)c2ccc3ccccc3n2)C1]

### Step 1:

To a solution of compound W3_1 (5 g, 30.56 mmol) and tert-butyl (3R)-3-aminopiperidine-1-carboxylate (6.12 g, 32.56 mmol, specific optical rotation=−35° at 20° C. (concentration=5 in MeOH)) in dioxane (150 mL) were added Cs₂CO₃ (19.92 g, 61.12 mmol), Pd(OAc)₂ (686.13 mg, 3.06 mmol) and BINAP (1.90 g, 3.06 mmol). The mixture was stirred at 100° C. for 16 h under N₂. The mixture was brown and muddy. After 16 h, the reaction mixture was filtered, and the filtrate was concentrated in vacuo to give a residue. The residue was diluted with EtOAc (100 mL), and the organic layer was washed with water (3×100 mL). The aqueous layer was extracted with EtOAc (3×100 mL). The combined organic layer was dried over Na₂SO₄, filtered, and concentrated under reduced pressure. The residue was purified by flash silica gel chromatography (Eluent of 0˜15% Ethyl acetate/Petroleum ether) to give compound W3_2 (5.6 g, 56%) as a yellow solid.

¹H NMR (400 MHZ, CD₃OD) δ 7.83 (d, J=9.0 Hz, 1H), 7.60 (dd, J=8.1, 13.4 Hz, 2H), 7.52-7.41 (m, 1H), 7.24-7.09 (m, 1H), 6.77 (d, J=8.9 Hz, 1H), 4.06 (dd, J=3.8, 7.5 Hz, 1H), 3.98-3.37 (m, 2H), 3.31-2.67 (m, 2H), 2.20-1.98 (m, 1H), 1.83 (s, 1H), 1.72-1.53 (m, 2H), 1.52-1.16 (m, 9H).

### Step 2:

A solution of compound W3_2 (4 g, 12.22 mmol) in THF (100 mL) was cooled to −78° C. under N₂, and then triethylamine (7.42 g, 73.30 mmol) and triphosgene (10.88 g, 36.65 mmol) were added. The mixture was stirred at 25° C. for 16 h. The mixture was yellow and muddy. The mixture was poured into ice water (30 mL) and extracted with EtOAc (4×100 mL). The combined organic layer was washed with saturated NaHCO₃ (50 mL), brine (50 mL), and then dried over Na₂SO₄ and filtered, concentrated. The crude product was purified by flash silica gel chromatography (Eluent of 0˜15% EtOAc/PE) to give compound WA003_1 (1 g, 21%) as a white solid.

¹H NMR (400 MHZ, CD₃OD) δ ppm 8.49 (d, J=8.5 Hz, 1H), 8.06 (dd, J=8.3, 13.8 Hz, 2H), 7.91-7.83 (m, 1H), 7.76-7.68 (m, 1H), 7.54 (d, J=8.5 Hz, 1H), 4.46 (td, J=2.0, 12.7 Hz, 1H), 4.28 (tt, J=3.9, 11.1 Hz, 1H), 3.92 (d, J=13.3 Hz, 1H), 3.24-2.87 (m, 1H), 2.64-2.45 (m, 1H), 2.19-2.04 (m, 1H), 1.72-1.35 (m, 12H).

### Experimental for Intermediate (WA004_1)

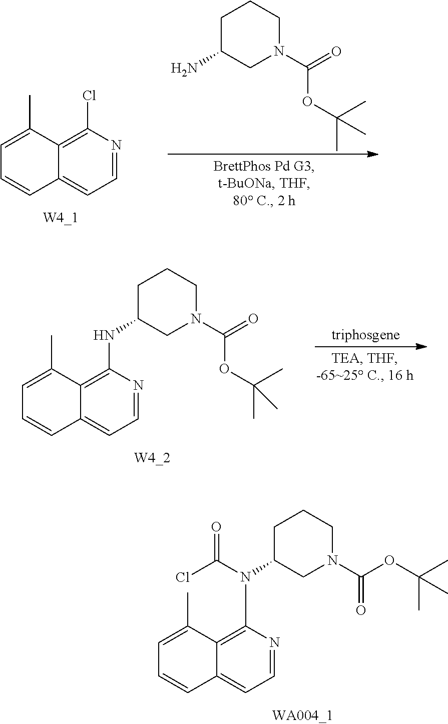 [SMILES: Cc1cccc2ccnc(Cl)c12 ; CC(C)(C)OC(=O)N1CCC[C@@H](N)C1 ; Cc1cccc2ccnc(N[C@@H]3CCCN(C(=O)OC(C)(C)C)C3)c12 ; Cc1cccc2ccnc(N(C(=O)Cl)[C@@H]3CCCN(C(=O)OC(C)(C)C)C3)c12]

### Step 1:

A solution of compound W4_1 (510 mg, 2.87 mmol), tert-butyl (3R)-3-aminopiperidine-1-carboxylate (862.53 mg, 4.32 mmol), t-BuONa (827.85 mg, 8.61 mmol) and BrettPhos Pd G3 (182.19 mg, 200.97 μmol) in THF (18 mL) was purged with N₂ and then heated to 80° C. for 2 h under N₂. The reaction mixture was poured into H₂O (60 mL). The mixture was extracted with ethyl acetate (3×60 mL), and the combined organic layer was dried over MgSO₄, filtered, and concentrated. The residue was purified by flash silica gel chromatography (Eluent of 5% THF/Petroleum ether) to give compound W4_2 (964 mg, 98%) as a yellow oil.

### Step 2:

A solution of compound W4_2 (964 mg, 2.82 mmol) in THF (14 mL) was cooled to −65° C., and then triethylamine (1.71 g, 16.94 mmol) and triphosgene (2.80 g, 9.44 mmol) were added. The mixture was allowed to slowly warm to 25° C., and then stirred at 25° C. for 16 h. The mixture was filtered. The filtrate was quenched with saturated NH₄Cl (200 mL) and extracted with ethyl acetate (3×100 mL). The combined organic layer was dried over MgSO₄, filtered, and concentrated. The residue was purified by flash silica gel chromatography (Eluent of 5% THF/Petroleum ether) to give key intermediate WA004_1 (790 mg, 69%) as a white solid.

### Experimental for Intermediate (WA006_1)

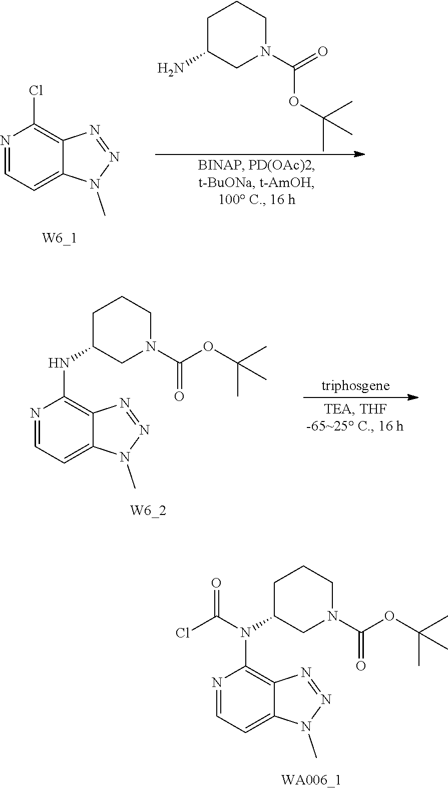 [SMILES: Cn1nnc2c(Cl)nccc21 ; CC(C)(C)OC(=O)N1CCC[C@@H](N)C1 ; Cn1nnc2c(N[C@@H]3CCCN(C(=O)OC(C)(C)C)C3)nccc21 ; Cn1nnc2c(N(C(=O)Cl)[C@@H]3CCCN(C(=O)OC(C)(C)C)C3)nccc21]

### Step 1:

To a solution of compound W6_1 (880 mg, 5.20 mmol) in t-AmOH (28 mL) was added tert-butyl (3R)-3-aminopiperidine-1-carboxylate (1.57 g, 7.84 mmol), t-BuONa (3.31 g, 34.44 mmol), BINAP (227.52 mg, 365.40 μmol), and Pd(Oac)₂ (82.04 mg, 365.40 μmol) under N₂. The reaction was heated to 100° C. for 16 h under N₂. The mixture was poured onto saturated aq. NH₄Cl (200 mL), and the product was extracted with EtOAc (180 mL). The organic layer was dried with MgSO₄, filtered, and concentrated. The residue was purified by flash silica gel chromatography (Eluent of 35% THF/Petroleum ether) to give W6_2 (847 mg, 49%) as a oil.

### Step 2:

A solution of compound W6_2 (847 mg, 2.55 mmol) in THF (12 mL) was cooled to −65° C., and then triethylamine (1.55 g, 15.29 mmol) and triphosgene (2.00 g, 6.74 mmol) were added. The mixture was allowed to slowly warm to 25° C., and then stirred at 25° C. for 16 h. The mixture was filtered. The filtrate was quenched with saturated aq. NH₄Cl (200 mL) and extracted with ethyl acetate (3×100 mL). The combined organic layers were dried over MgSO₄, filtered, and concentrated. The residue was purified by flash silica gel chromatography (Eluent of 35% THF/Petroleum ether) to give key intermediate WA006_1 (370 mg, 37%) as a white solid.

### Experimental for Intermediate (WA007_1)

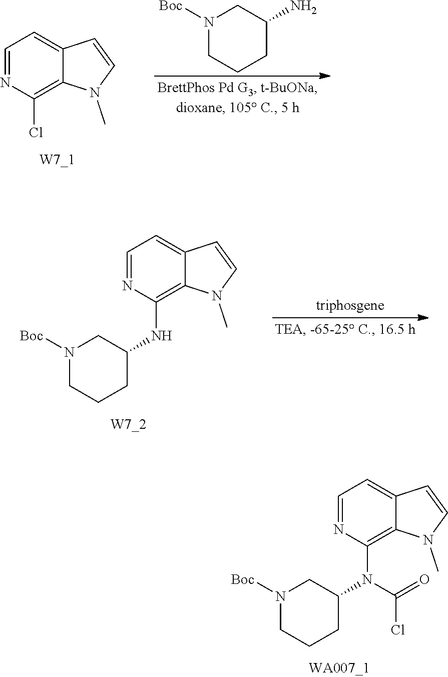 [SMILES: Cn1ccc2ccnc(Cl)c21 ; CC(C)(C)OC(=O)N1CCC[C@@H](N)C1 ; Cn1ccc2ccnc(N[C@@H]3CCCN(C(=O)OC(C)(C)C)C3)c21 ; Cn1ccc2ccnc(N(C(=O)Cl)[C@@H]3CCCN(C(=O)OC(C)(C)C)C3)c21]

### Step 1:

A mixture of compound W7_1 (2.69 g, 16.15 mmol), tert-butyl (3R)-3-aminopiperidine-1-carboxylate (6.47 g, 32.29 mmol), t-BuONa (3.10 g, 32.29 mmol) in dioxane (100 mL) was purged with N₂ for 2 minutes. Then BrettPhos Pd G₃ (439.08 mg, 484.37 mmol) was added under N₂. The resulting mixture was stirred at 105° C. for 5 h. Water (50 mL) was added to the mixture, and the product was extracted with EtOAc (2×100 mL). The combined organic layer was dried over MgSO₄, filtered, and concentrated. The residue was purified by flash silica gel chromatography (Eluent of 0˜80% Tetrahydrofuran/Petroleum ether) to give compound W7_2 (5 g, 91%) as a yellow oil.

¹H NMR (400 MHZ, CD₃OD) δ ppm 8.24 (s, 1H), 7.27 (d, J=2.9 Hz, 1H), 6.72 (d, J=0.6 Hz, 1H), 6.26 (d, J=2.8 Hz, 1H), 3.81 (s, 3H), 3.63-3.52 (m, 1H), 3.26-3.00 (m, 2H), 2.79 (t, J=11.2 Hz, 1H), 2.58 (br s, 1H), 2.07-2.06 (m, 1H), 1.99-1.89 (m, 1H), 1.81-1.79 (m, 1H), 1.48-1.47 (m, 10H).

### Step 2:

A solution of compound W7_2 (2 g, 6.05 mmol) in THF (80 mL) was cooled to −65° C. under N₂, and then triethylamine (3.67 g, 36.32 mmol) was added. The mixture was stirred at −65° C. for 30 min. Then triphosgene (6 g, 20.22 mmol) was added to the mixture. The mixture was allowed to warm to 25° C. and then stirred for 16 h. The mixture was poured onto ice water (40 mL) and extracted with EtOAc (200 mL). The organic layer was combined, washed with saturated NaHCO₃ (50 mL) and brine (50 mL), dried over Na₂SO₄, filtered, and concentrated. The crude product was purified by flash silica gel chromatography (Eluent of 0˜60% EtOAc/dichloromethane) to give key intermediate WA007_1 (381 mg, 14%) as a yellow oil.

¹H NMR (400 MHZ, CD₃OD) δ ppm 8.72 (s, 1H), 7.63-7.56 (m, 2H), 6.66 (d, J=3.0 Hz, 1H), 4.45-4.42 (m, 1H), 4.30-4.18 (m, 1H), 4.01 (s, 3H), 3.97-3.89 (m, 1H), 2.86 (br s, 1H), 2.52 (br s, 1H), 2.12-2.09 (m, 1H), 1.76-1.62 (m, 2H), 1.53-1.48 (m, 10H).

### Experimental for Intermediate (WA008_1)

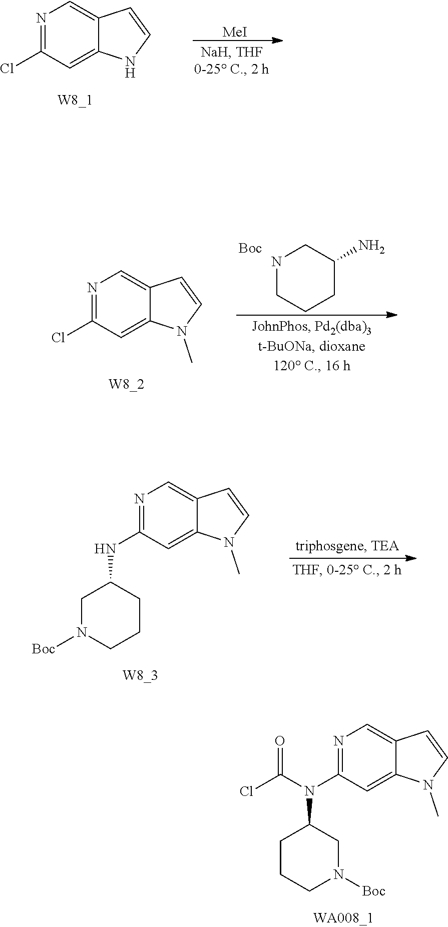

### Step 1:

A solution of compound W8_1 (5 g, 32.77 mmol) in THF (30 mL) was cooled to 0° C. under N₂ and then NaH (1.57 g, 39.32 mmol, 60% purity) was added in portions. The mixture was stirred at 0° C. for 30 min. Then, Mel (5.12 g, 36.05 mmol, 2.24 mL) was added dropwise. After the addition, the mixture was stirred at 25° C. for 2 h. The mixture was poured onto saturated NH₄Cl (25 mL) and extracted with EtOAc (3×20). The combined organic layer was washed with brine (20 mL), dried over Na₂SO₄, filtered, and concentrated. Compound W8_2 (5.92 g, 98%) was obtained as a solid. The product was used without further purification.

### Step 2:

Pd₂(dba)₃ (742 mg, 810.29 μmol), t-BuONa (3.11 g, 32.41 mmol), and JohnPhos (241.79 mg, 810.29 μmol) were added to a solution of compound W8_2 (3 g, 16.21 mmol) and tert-butyl (3R)-3-aminopiperidine-1-carboxylate (3.57 g, 17.83 mmol) in dioxane (30 mL). The reaction mixture was stirred at 120° C. for 16 h under N₂. The mixture was poured onto ice water (20 mL) and extracted with EtOAc (2×15 mL). The combined organic layer was washed with brine (10 mL), dried over Na₂SO₄, filtered, and concentrated. The residue was purified by flash silica gel chromatography (Eluent of 40% Ethyl acetate/Petroleum ether) to give compound W8_3 (1.5 g, 26%) as a brown oil.

### Step 3:

A mixture of compound W8_3 (1.45 g, 4.39 mmol) and triethylamine (2.66 g, 26.33 mmol) in THF (20 mL) was cooled to 0° C., and then triphosgene (5.11 g, 17.22 mmol) was added. The mixture was stirred at 25° C. for 2 h under N₂. The mixture was poured onto saturated aq. NaHCO₃ (100 mL) and the product was extracted with EtOAc (2×100 mL). The combined organic layer was washed with brine (50 mL), dried over Na₂SO₄, filtered, and concentrated. The residue was purified by flash silica gel chromatography (Eluent of 35% THF/Petroleum ether) to give compound WA008_1 (1.1 g, 61%) as a yellow solid.

¹H NMR (400 MHZ, DMSO-d₆) δ ppm 8.75 (s, 1H), 7.66 (s, 1H), 7.57 (d, J=3.1 Hz, 1H), 6.68 (d, J=2.6 Hz, 1H), 4.29 (br d, J=10.5 Hz, 1H), 4.18-4.06 (m, 1H), 3.86 (s, 3H), 3.79 (br d, J=13.0 Hz, 1H), 2.44 (br s, 2H), 2.06-1.98 (m, 1H), 1.59 (br d, J=12.6 Hz, 1H), 1.47-1.33 (m, 11H).

### Experimental for Intermediate (WA009_1)

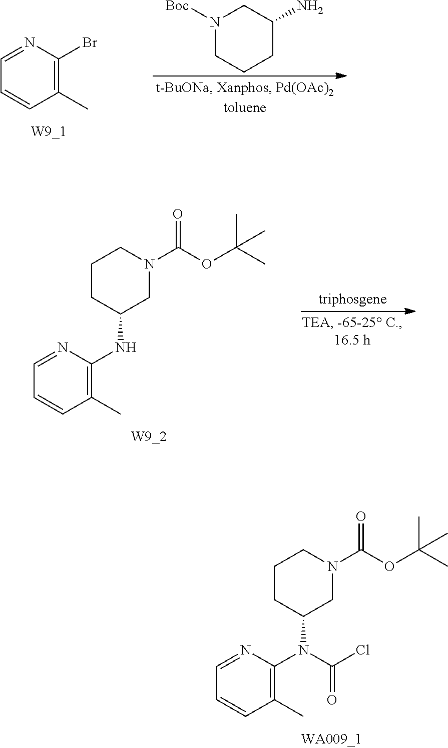

### Step 1:

t-BuONa (5.59 g, 58.13 mmol), Xantphos (840.91 mg, 1.45 mmol), and Pd(OAc)₂ (326.28 mg, 1.45 mmol) were added to a solution of W9_1 (5 g, 29.07 mmol) and tert-butyl (3R)-3-aminopiperidine-1-carboxylate (5.82 g, 29.07 mmol) in toluene (100 mL). The resulting mixture was purged with N₂ 2 times, and then stirred at 100° C. for 6 h. The mixture was cooled to 25° C. and diluted with water (80 mL). The aqueous layer was extracted with EtOAc (3×50 mL). The combined organic layer was washed with brine (2×50 mL), dried over Na₂SO₄, filtered, and concentrated under reduced pressure. The residue was purified by flash silica gel chromatography (Eluent of 0˜23% Ethyl acetate/Petroleum ether) to give Compound W9_2 (5.34 g, 63%) as a yellow solid.

¹H NMR (400 MHZ, DMSO-d₆) δ ppm 7.87 (dd, J=1.4, 5.0 Hz, 1H), 7.31-7.13 (m, 1H), 6.47 (dd, J=5.0, 7.0 Hz, 1H), 5.37 (brs, 1H), 4.05-3.66 (m, 3H), 2.99-2.66 (m, 2H), 2.04 (s, 3H), 1.96-1.87 (m, 1H), 1.77-1.66 (m, 1H), 1.56 (s, 1H), 1.46-1.23 (m, 10H).

### Step 2:

A solution of W9_2 (1 g, 3.43 mmol) in THF (10 mL) was cooled to −65° C. and triethylamine (2.08 g, 20.59 mmol) and triphosgene (3.00 g, 10.11 mmol) were added to the solution. The yellow mixture was allowed to slowly warm to 25° C. and then stirred at 25° C. for 18 h. The mixture was poured onto ice water (200 mL) and extracted with EtOAc (3×100 mL). The combined organic layer was washed with saturated aq. NaHCO₃ (50 mL), brine (50 mL), dried over Na₂SO₄, filtered, and concentrated. The residue was purified by flash silica gel chromatography (Eluent of 0˜27% Ethyl acetate/Petroleum ether) to give the key intermediate WA009_1 (0.92 g, 73%) as a light yellow solid.

¹H NMR (400 MHZ, CD₃OD) δ ppm 8.41 (br d, J=4.4 Hz, 1H), 7.86 (m, 1H), 7.44 (br d, J=7.5 Hz, 1H), 4.62-4.26 (m, 1H), 4.22-3.84 (m, 2H), 3.16 (s, 1H), 2.73-2.46 (m, 1H), 2.40 (d, J=8.3 Hz, 3H), 1.91 (bs, 1H), 1.78-1.59 (m, 1H), 1.58-1.52 (m, 1H), 1.48 (d, J=16.5 Hz, 10H).

### Experimental for Intermediate (WA010_1)

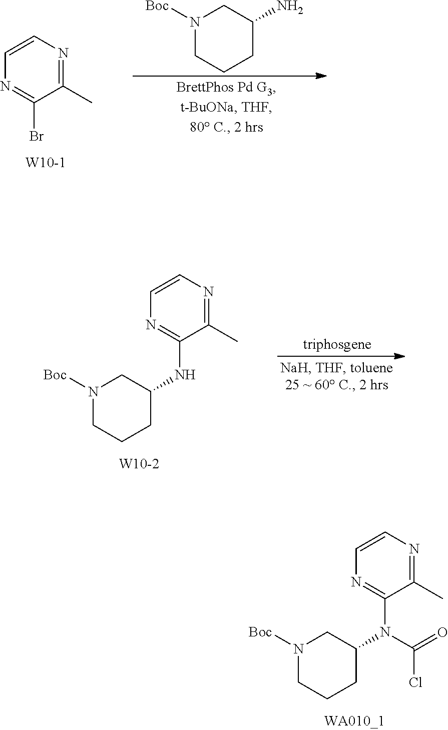

### Step 1:

Compound W10-1 (5 g, 39 mmol) and tert-butyl (3R)-3-aminopiperidine-1-carboxylate (11.7 g, 58.3 mmol) were dissolved in THF (100 mL), and t-BuONa (11.2 g, 116.7 mmol) and BrettPhos Pd G3 (1.76 g, 1.94 mmol) were added. The reaction was purged with N₂ for 2 minutes and then heated to 80° C. for 2 h. The reaction mixture was filtered and concentrated under reduced pressure. The residue was purified by flash silica gel chromatography (Eluent of 30% Ethyl acetate/Petroleum ether) to give compound W10-2 (9 g, 79.2%) as a brown oil.

### Step 2:

NaH (3.69 g, 92.4 mmol, 60% purity) was added to a solution of compound W10-2 (9 g, 30.8 mmol) in THF (100 mL) under N₂. The reaction mixture was stirred at 25° C. for 0.5 h. Then, triphosgene (10.81 g, 36.43 mmol) in toluene (30 mL) was slowly added, after which the reaction mixture was stirred at 60° C. for 2 h. The reaction was quenched with saturated aqueous sodium bicarbonate (200 mL) and extracted with EtOAc (3×200 mL). The combined organic layer was dried over Na₂SO₄ and concentrated. The residue was purified by flash silica gel chromatography (Eluent of 40% Ethyl acetate/Petroleum ether) to give WA010_1 (2.6 g, 24%) as a yellow oil.

¹H NMR (400 MHZ, DMSO-d₆) δ ppm 8.73-8.64 (m, 1H), 8.54 (s, 1H), 5.75 (s, 1H), 4.38-3.93 (m, 2H), 3.92-3.59 (m, 1H), 2.56 (br s, 3H), 2.09-1.82 (m, 1H), 1.80-1.50 (m, 2H), 1.38 (d, J=18.8 Hz, 11H).

### Experimental for Intermediate (WA011_1)

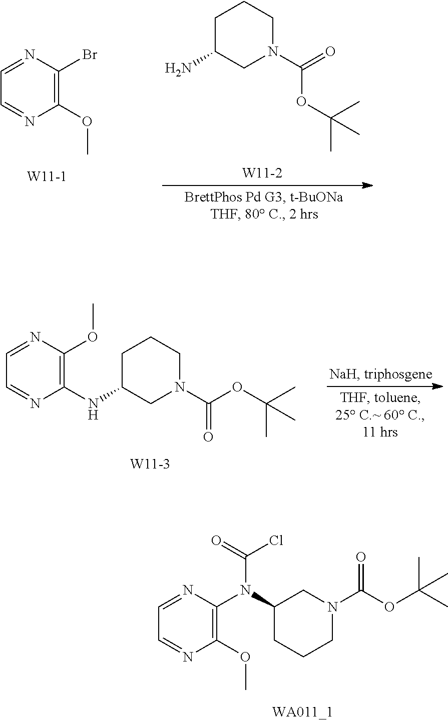

### Step 1:

To a solution of compound W11-1 (5.0 g, 34.59 mmol) and tert-butyl (3R)-3-aminopiperidine-1-carboxylate (8.31 g, 41.51 mmol) in THF (70 mL) was added t-BuONa (9.97 g, 103.76 mmol) and BrettPhos Pd G3 (1.57 g, 1.73 mmol). The reaction mixture was purged with N₂ for 2 minutes and then heated to 80° C. for 2 h under N₂. The reaction mixture was filtered, and the organic layer was concentrated under reduced pressure. The crude product was poured onto H₂O (150 mL) and extracted with ethyl acetate (3×150 mL). The combined organic layer was dried over anhydrous Na₂SO₄, and then concentrated in vacuo. The residue was purified by flash silica gel chromatography (Eluent of 18% Ethyl acetate/Petroleum ether) to give W11-3 (10 g, 84.4%) as yellow oil.

### Step 2:

NaH (1.63 g, 40.9 mmol, 60% purity) was added to a solution of compound W11-3 (10 g, 29.2 mmol) in THF (100 mL) at 25° C. under N₂. The mixture was stirred at 25° C. for 0.5 h. Then bis(trichloromethyl) carbonate (6.91 g, 23.29 mmol) in toluene (30 mL) was slowly added to the reaction mixture, which was then stirred at 60° C. for 8 h. LC-MS showed that ˜40% of compound W11-3 remained, so another batch of NaH (1.63 g, 40.87 mmol, 60% purity) was added to the reaction mixture. The mixture was stirred at 25° C. for 0.5 h, and then bis(trichloromethyl) carbonate (8.0 g, 26.97 mmol) in toluene (30 mL) was slowly added. The reaction mixture was stirred at 60° C. for another 2 h, after which LC-MS showed that the reaction was complete. The reaction mixture was quenched with saturated aqueous sodium bicarbonate (300 mL) and extracted with ethyl acetate (3×250 mL). The combined organic layer was dried over Mg₂SO₄ and concentrated. The residue was purified by flash silica gel chromatography (ISCO®; 120 g SepaFlash® Silica Flash Column, Eluent of 2% Ethyl acetate/dichloromethane gradient @ 100 mL/min) to give WA011_1 (3.5 g, 8.12 mmol, 27.81%, 86% purity) as a colorless oil.

¹H NMR (400 MHZ, DMSO-d₆) δ ppm 8.42 (d, J=2.1 Hz, 1H), 8.26 (d, J=2.5 Hz, 1H), 4.27-4.06 (m, 2H), 4.01 (s, 3H), 3.83-3.66 (m, 1H), 3.03-2.58 (m, 1H), 2.11-1.80 (m, 1H), 1.74-1.52 (m, 2H), 1.39 (br s, 10H), 1.28-1.06 (m, 1H).

### Experimental for Intermediate (WA012_1)

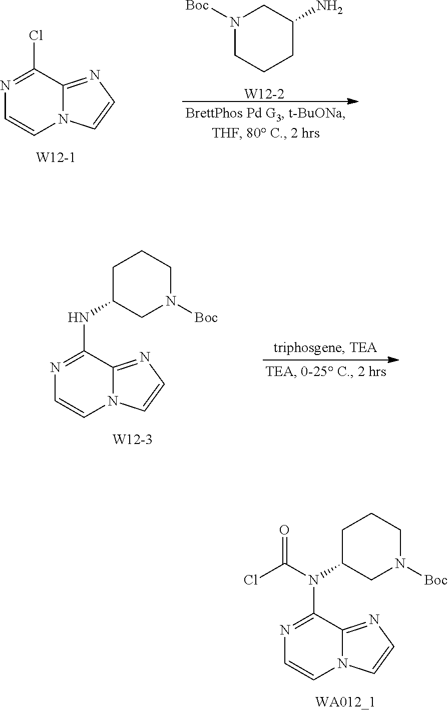

### Step 1:

t-BuONa (8.14 g, 84.65 mmol) and BrettPhos Pd G3 (3.07 g, 3.39 mmol) were added to a solution of compound W12-1 (6.5 g, 42.33 mmol) and compound W12-2 (9.32 g, 46.56 mmol) in THF (80 mL) under N₂. Then the reaction mixture was stirred at 80° C. for 2 h under N₂, after which it was diluted with water (200 mL) and extracted with EtOAc (3×200 mL). The combined organic layer was washed with saturated NaCl (200 mL), dried over Na₂SO₄, filtered, and concentrated under reduced pressure. The residue was purified by flash silica gel chromatography (Eluent of 30% THF/Petroleum ether) to give compound W12-3 (13.00 g, 75%) as a black solid.

### Step 2:

A solution of compound W12-3 (8 g, 19.66 mmol) and triethylamine (11.94 g, 117.96 mmol) in THF (140 mL) was cooled to 0° C., and triphosgene (17.11 g, 57.66 mmol) was added. The reaction mixture was stirred at 25° C. for 2 h under N₂. The mixture was filtered and washed with EtOAc (2×20 mL). The filtrate was diluted with saturated NH₄Cl (150 mL) and extracted with EtOAc (3×150 mL). The combined organic layer was washed with saturated aq. NaHCO₃ (200 mL), brine (200 mL), dried over Na₂SO₄, filtered, and concentrated. The residue was purified by flash silica gel chromatography (Eluent of 43% THF/Petroleum ether) to give WA012_1 (3.75 g, 44%) as a yellow solid.

¹H NMR (400 MHZ, DMSO-d₆) δ ppm 8.78 (d, J=4.8 Hz, 1H), 8.30 (d, J=0.8 Hz, 1H), 7.97-7.85 (m, 2H), 4.30 (br d, J=11.8 Hz, 1H), 4.18 (br t, J=10.9 Hz, 1H), 3.75 (br d, J=11.0 Hz, 1H), 3.39 (br s, 2H), 1.98 (br d, J=15.1 Hz, 1H), 1.67-1.53 (m, 1H), 1.38 (s, 10H), 1.36 (s, 1H).

### 2.2 Experimental Details for Final Compound Synthesis Using the Procedures in 2.1

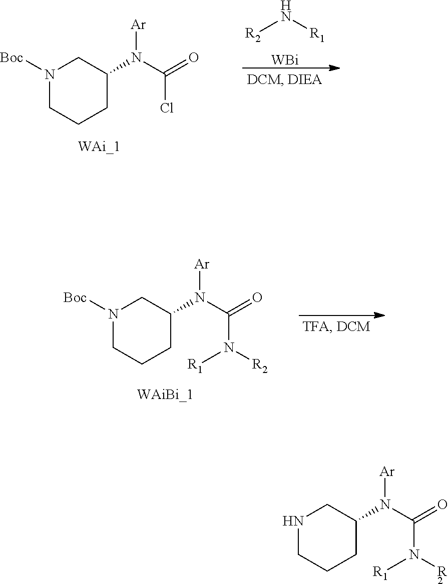

### Procedure W5

### Step 1

DIEA (150 μmol, 3.0 eq) was added to a solution of WBi (60 μmol, 1.2 eq) and WAi_1 (18.7 mg, 50 μmol, 1.0 eq) in dichloromethane (100 μL). The reaction vial was capped and shaken at 50° C. for 16 h. The reaction mixture was concentrated under reduced pressure, and the residue was diluted with H₂O (1.0 mL) and extracted with dichloromethane (3×1.5 mL). The combined organic layer was concentrated under reduced pressure to give WAiBi_1, which was used in the next step directly.

### Step 2:

WAiBi_1 (˜50 μmol, 1.0 eq) was dissolved in a mixed solution (1.0 mL) of dichloromethane:TFA (v/v=5:1). The reaction mixture was shaken at 30° C. for 2 h. The reaction mixture was concentrated under reduced pressure to give a residue, which was purified by preparative HPLC to obtain the final product.

### 3.1 Experimental Details for Intermediate Synthesis

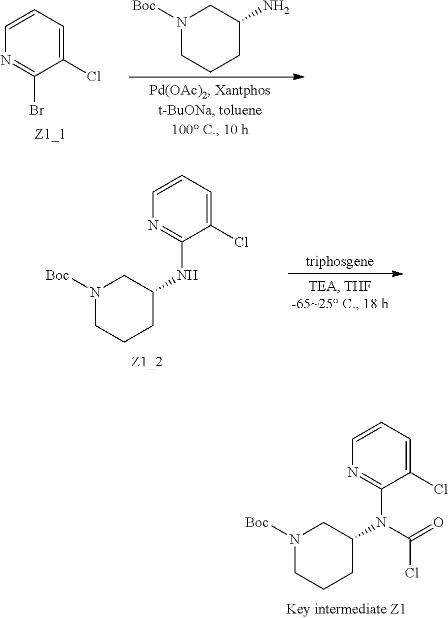 [SMILES: Clc1cccnc1Br ; CC(C)(C)OC(=O)N1CCC[C@@H](N)C1 ; CC(C)(C)OC(=O)N1CCC[C@@H](Nc2ncccc2Cl)C1 ; CC(C)(C)OC(=O)N1CCC[C@@H](N(C(=O)Cl)c2ncccc2Cl)C1]

### Step 1:

A solution of compound Z1_1 (25 g, 130 mmol), tert-butyl (3R)-3-aminopiperidine-1-carboxylate (26.0 g, 130 mmol), t-BuONa (24.9 g, 260 mmol), Xantphos (3.76 g, 6.50 mmol), and Pd(Oac)₂ (1.46 g, 6.50 mmol) in toluene (300 mL) was purged with N₂ for 2 min and then heated at 100° C. for 10 h under N₂. The reaction mixture was poured into H₂O (800 mL) and extracted with ethyl acetate (3×800 mL). The combined organic layer was washed with brine (500 mL), dried over anhydrous Na₂SO₄, and concentrated in vacuo. The residue was purified by flash silica gel chromatography (Eluent of 18% Ethyl acetate/Petroleum ether) to give compound Z1_2 (35 g, 106 mmol, 82.1%, 95% purity) as a white solid.

¹H NMR (400 MHZ, DMSO-d₆) δ ppm 8.00 (dd, J=4.88, 1.50 Hz, 1H), 7.61 (dd, J=7.63, 1.50 Hz, 1H), 6.59 (dd, J=7.63, 4.88 Hz, 1H), 5.89 (br s, 1H), 3.87-3.99 (m, 1H), 3.40-3.85 (m, 2H), 2.74-3.25 (m, 2H), 1.82-1.92 (m, 1H), 1.66 (br dd, J=10.44, 3.19 Hz, 2H), 1.23-1.45 (m, 10H).

### Step 2:

A solution of compound Z1_2 (12 g, 38.5 mmol) in THF (150 mL) was cooled to −65° C., and then triethylamine (23.3 g, 231 mmol) and triphosgene (34.2 g, 115 mmol) were added. Then the reaction mixture was allowed to slowly warm to 25° C. and subsequently stirred at 25° C. for 18 h. The reaction mixture was filtered, and the filtrate was quenched with saturated NH₄Cl (300 mL) and extracted with ethyl acetate (3×300 mL). The combined organic layer was dried over MgSO₄, filtered, and concentrated. The residue was purified by flash silica gel chromatography (Eluent of 20% Ethyl acetate/Petroleum ether) to give key intermediate Z1 (11.5 g, 75.8%) as a colorless oil.

¹H NMR: (400 MHZ, CDCl₃) δ ppm 8.12 (d, J=5.38 Hz, 1H), 7.58 (d, J=5.25 Hz, 1H), 6.57 (d, J=3.00 Hz, 1H), 4.66 (br d, J=12.26 Hz, 1H), 4.18-4.36 (m, 1H), 3.91-4.05 (m, 4H), 3.33 (br t, J=11.13 Hz, 1H), 2.46 (br s, 1H), 1.65-1.81 (m, 1H), 1.48 (br s, 9H).

### General Procedure for Preparation of Compound Z2

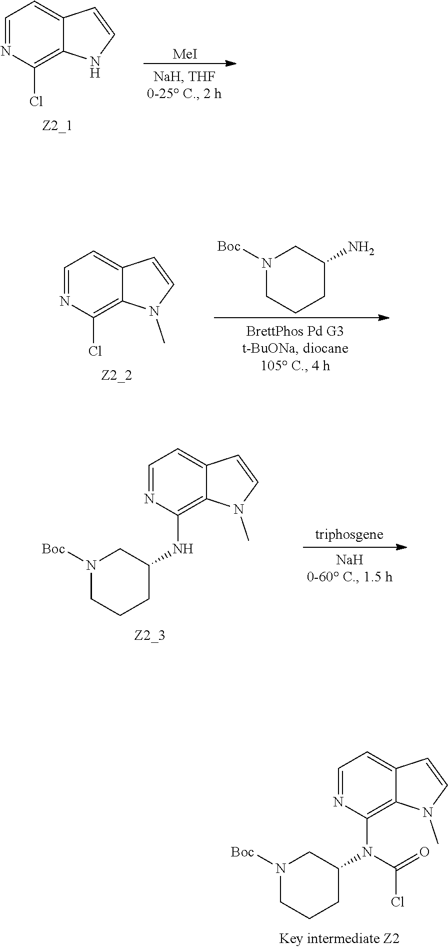 [SMILES: "MeI" ; Clc1nccc2cc[nH]c12 ; Cn1ccc2ccnc(Cl)c21 ; CC(C)(C)OC(=O)N1CCC[C@@H](N)C1 ; Cn1ccc2ccnc(N[C@@H]3CCCN(C(=O)OC(C)(C)C)C3)c21 ; Cn1ccc2ccnc(N(C(=O)Cl)[C@@H]3CCCN(C(=O)OC(C)(C)C)C3)c21]

### Step 1:

To a solution of compound Z2_1 (25.0 g, 164 mmol) in THF (200 mL) was added NaH (7.86 g, 196 mmol, 60% purity) in portions at 0° C. under N₂. The reaction mixture was stirred at 0° C. for 30 min. Then Mel (25.6 g, 180 mmol) was added dropwise. After the addition, the reaction mixture was stirred at 25° C. for 2 h. The reaction was poured into saturated NH₄Cl (200 mL) and extracted with EtOAc (200 mL). The organic layer was washed with brine (3×50 mL), dried over Na₂SO₄, filtered, and concentrated to give compound Z2_2 (26 g, 95%) as a yellow solid. The product was used without further purification.

¹H NMR (400 MHZ, CDCl₃) δ ppm 7.94 (d, J=5.38 Hz, 1H), 7.41 (d, J=5.38 Hz, 1H), 7.16 (d, J=3.13 Hz, 1H), 6.48 (d, J=3.13 Hz, 1H), 4.15 (s, 3H).

### Step 2:

A mixture of compound Z2_2 (15.0 g, 90.0 mmol), tert-butyl (3R)-3-aminopiperidine-1-carboxylate (19.8 g, 99.0 mmol), and t-BuONa (17.3 g, 180 mmol) in dioxane (300 mL) was purged with N₂ for 2 minutes. Then, BrettPhos Pd G3 (2.45 g, 2.70 mmol) was added under N₂. The resulting mixture was stirred at 105° C. for 4 h. The reaction mixture was poured into ice water (500 mL) and extracted with EtOAc (3×200 mL). The combined organic layer was washed with brine (3×100 mL), dried over Na₂SO₄, filtered, and concentrated to give compound Z2_3 (29 g, 97%) as a yellow solid. The product was used without further purification.

¹H NMR (400 MHZ, CDCl₃) δ ppm 7.69 (d, J=5.77 Hz, 1H), 6.92 (d, J=3.01 Hz, 1H), 6.88 (d, J=5.77 Hz, 1H), 6.32 (d, J=3.01 Hz, 1H)), 4.37 (br s, 1H), 4.08 (s, 3H), 3.85-3.95 (m, 1H), 3.77-3.84 (m, 1H), 3.70 (s, 3H), 3.42 (br d, J=13.30 Hz, 1H), 3.06-3.17 (m, 1H), 1.89 (br s, 1H), 1.61 (br s, 1H), 1.45 (s, 9H).

### Step 3:

To a solution of compound Z2_3 (10 g, 30.2 mmol) in THF (150 mL) was added NaH (1.57 g, 39.3 mmol, 60% purity) under N₂ at 0° C. The mixture was stirred at 25° C. for 30 min. Then a solution of triphosgene (6.44 g, 21.70 mmol) in toluene (20 mL) was added dropwise. The reaction mixture was stirred at 60° C. for 1 h. The reaction mixture was poured into ice water (200 mL) and extracted with EtOAc (100 mL). The combined organic layer was washed with saturated aq. NaHCO₃ (50 mL), brine (50 mL), dried over Na₂SO₄, filtered, and concentrated. The crude product was purified by flash silica gel chromatography (Eluent of 0˜20% Ethyl acetate/Petroleum ether) to give key intermediate Z2 (5.2 g, 41%) as a yellow gum.

¹H NMR (400 MHZ, CDCl₃) δ ppm 8.12 (d, J=5.38 Hz, 1H), 7.58 (d, J=5.25 Hz, 1H), 7.21 (d, J=2.88 Hz, 1H), 6.57 (d, J=3.00 Hz, 1H), 4.66 (br d, J=12.26 Hz, 1H), 4.18-4.36 (m, 1H), 3.91-4.05 (m, 4H), 3.33 (br t, J=11.13 Hz, 1H), 2.46 (br s, 1H), 1.65-1.81 (m, 1H), 1.48 (br s, 9H), 1.00-1.20 (m, 1H), 0.78-0.94 (m, 2H).

### General Procedure for Preparation of Compound ZB002

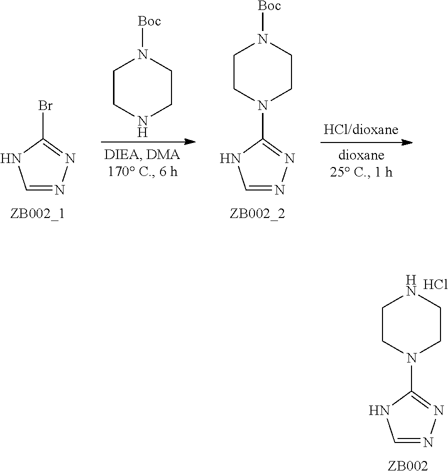 [SMILES: Brc1nnc[nH]1 ; CC(C)(C)OC(=O)N1CCNCC1 ; CC(C)(C)OC(=O)N1CCN(c2nnc[nH]2)CC1 ; Cl ; c1nnc(N2CCNCC2)[nH]1]

### Step 1:

DIEA (1.75 g, 13.5 mmol) and N-Boc piperazine (1.26 g, 6.76 mmol) were added to a solution of compound ZB002_1 (1.00 g, 6.76 mmol) in DMA (20.0 mL). The reaction mixture was stirred at 170° C. for 6 h, after which it was poured into saturated NaHCO₃ (50 mL) and extracted with EtOAc (3×50 mL). The combined organic layer was concentrated in vacuo. The residue was purified by flash silica gel chromatography (Eluent of 47% THF/petroleum ether) to give compound ZB002_2 (240 mg, 14%) as a yellow oil.

¹H NMR (400 MHZ, DMSO-d₆) δ ppm 13.12 (br s, 1H). 8.18 (s, 1H). 3.40 (br s, 4H). 3.25 (m, 4H). 1.41 (s, 9H).

### Step 2:

HCl/dioxane (4.00 M, 5.00 mL) was added to a solution of compound ZB002_2 (240 mg, 947 μmol) in dioxane (2.00 mL). The reaction mixture was stirred at 25° C. for 1 h. The reaction was then concentrated in vacuo to give compound ZB002 hydrochloride salt (167 mg, 92.9%) as a yellow solid, which was directly used in the next step without further purification.

¹H NMR (400 MHz, DMSO-d₆) δ ppm 9.65 (br s, 2H). 8.52 (s, 1H). 3.74 (br d, J=4.52 Hz, 4H). 3.21 (br s, 4H).

### General Procedure for Preparation of Compound ZB011

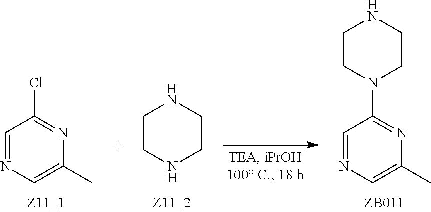

Triethylamine (2.36 g, 23.3 mmol, 3.25 mL) was added to a solution of Z11_1 (1 g, 7.78 mmol) and Z11_2 (670 mg, 7.78 mmol) in i-PrOH (10 mL). The reaction mixture was stirred at 100° C. for 18 h, after which it was filtered and concentrated in vacuo. The crude product was used in the next step directly. Compound ZB011 (800 mg, crude) was obtained as a yellow oil.

¹H NMR (400 MHZ, DMSO-d₆) δ ppm 8.05 (s, 1H). 7.70 (s, 1H). 3.68 (m, 1H). 3.44 (m, 4H). 2.77 (dd, J=5.93, 4.22 Hz, 4H). 2.29 (s, 3H).

### General Procedure for Preparation of Compound Z14

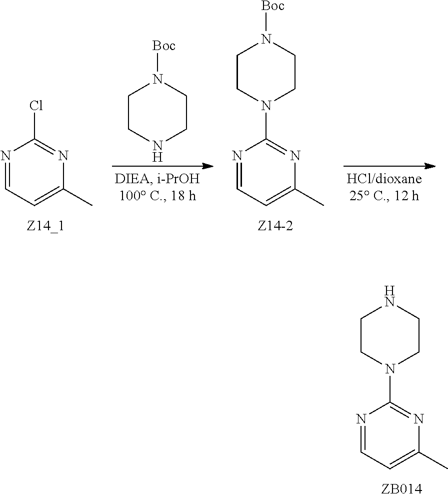

### Step 1:

DIEA (1.51 g, 11.7 mmol, 2.03 mL) was added to a solution of compound Z14-1 (500 mg, 3.89 mmol) and tert-butyl piperazine-1-carboxylate (869 mg, 4.67 mmol) in i-PrOH (10.0 mL). The reaction mixture was stirred at 25° C. for 2 h, after which it was poured into water (20 mL) and extracted with ethyl acetate (3×20 mL). The combined organic layer was concentrated in vacuo to give compound Z14-2 (500 mg, crude) as a white residue which was used without further purification.

### Step 2:

To a solution of compound Z14-2 (453 mg, 1.63 mmol) in dioxane (3.00 mL) was added HCl/dioxane (4 M, 406.96 μL). The reaction mixture was stirred at 25° C. for 12 h. The mixture was then concentrated in vacuo to give compound ZB014 hydrochloride salt (330 mg) as a gray solid which was used without further purification.

¹H NMR (400 MHZ, DMSO-d₆) δ ppm 8.30 (d, J=5.1 Hz, 1H), 6.71 (d, J=5.1 Hz, 1H), 4.05-3.97 (m, 4H), 3.34 (br s, 1H), 3.17-3.10 (m, 4H), 2.38-2.33 (m, 3H), 1.33-1.29 (m, 1H).

### General Procedure for Preparation of Compound ZB021

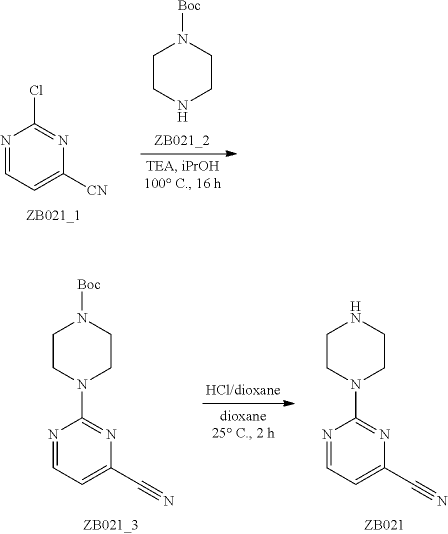 [SMILES: N#Cc1ccnc(Cl)n1 ; CC(C)(C)OC(=O)N1CCNCC1 ; CC(C)(C)OC(=O)N1CCN(c2nccc(C#N)n2)CC1 ; N#Cc1ccnc(N2CCNCC2)n1]

### Step 1:

To a solution of compound ZB021_1 (340 mg, 2.44 mmol) and compound ZB021_2 (453 mg, 2.44 mmol) in i-PrOH (5.00 mL) was added DIEA (629 mg, 4.87 mmol). The reaction mixture was stirred at 100° C. for 16 h, after which it was concentrated under reduced pressure. The residue was purified by flash silica gel chromatography (Eluent of 0˜50% Ethyl acetate/Petroleum ether, gradient). Compound ZB021_3 (700 mg, 99.3%) was obtained as a yellow solid. The product was used without further purification.

¹H NMR (400 MHZ, DMSO-d₆) δ ppm 8.67 (d, J=4.6 Hz, 1H), 7.19 (d, J=4.6 Hz, 1H), 3.76-3.70 (m, 4H), 3.45-3.39 (m, 4H), 1.43 (s, 9H).

### Step 2:

To a solution of compound ZB021_3 (300 mg, 1.04 mmol) in dioxane (2.00 mL) was added HCl/dioxane (4 M, 5 mL). The reaction mixture was stirred at 25° C. for 2 h, after which it was concentrated under reduced pressure. The crude product was used in the next step without further purification. Compound ZB021 hydrochloride salt (1.26 g, crude) was obtained as a yellow solid.

¹H NMR (400 MHZ, DMSO-d₆) δ ppm 9.55-9.31 (m, 3H), 8.73 (d, J=4.6 Hz, 1H), 7.29 (d, J=4.8 Hz, 1H), 4.03-3.95 (m, 4H), 3.16 (br s, 5H).

### General Procedure for Preparation of Compound ZB023

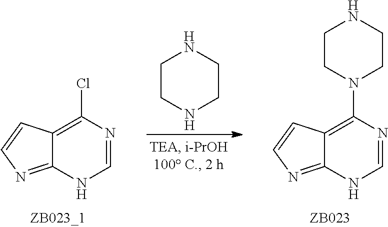 [SMILES: Clc1nc[nH]c2nccc1-2 ; C1CNCCN1 ; c1cc2c(N3CCNCC3)nc[nH]c-2n1]

Triethylamine (1.98 g, 19.5 mmol) and piperazine (729 mg, 8.47 mmol) were added to a solution of compound ZB023_1 (1.00 g, 6.51 mmol) in i-PrOH (15 mL). The reaction mixture was stirred at 100° C. for 2 h, after which it was filtrated and concentrated. The crude residue was triturated with EtOAc (20 mL) at 25° C. for 1 h and then filtered. The filter cake was dried to give compound ZB023 (293 mg, 22%) as a white solid. This compound was used without further purification.

¹H NMR (400 MHz, DMSO-d₆) δ ppm 11.72 (br s, 1H). 8.15 (m, 1H). 7.19 (br s, 1H). 6.63 (m, 1H). 3.86 (br s, 2H). 3.47 (m, 4H). 2.91 (br s, 3H).

LC-MS [M+H]⁺=204.2.

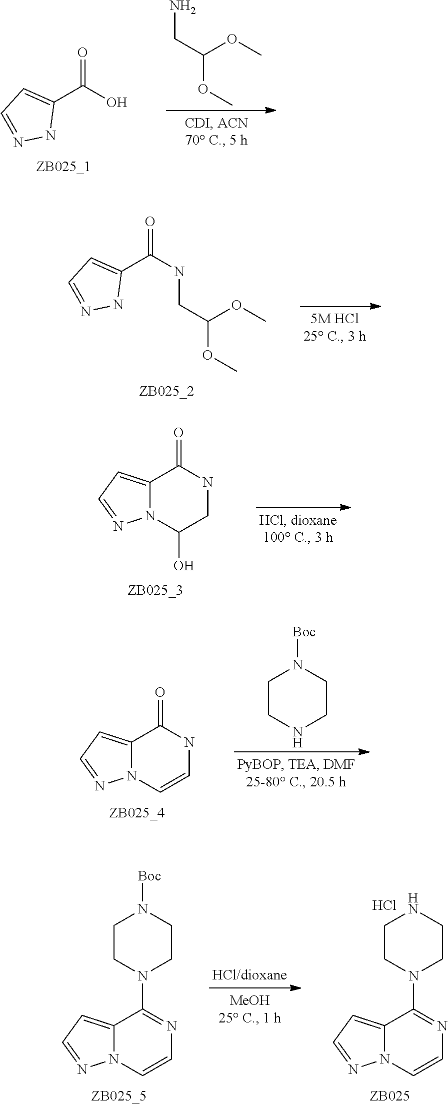

### Step 1:

Carbonyldiimidazole (8.68 g, 53.5 mmol) was added to a solution of compound WB025_1 (5.00 g, 44.6 mmol) in MeCN (100 mL). The reaction mixture was stirred at 70° C. for 3 h. Then 2,2-dimethoxyethanamine (4.69 g, 44.6 mmol) was added to the reaction. The reaction was stirred at 70° C. for 2 h, after which the solvent was removed by evaporation. The residue was dissolved in EtOAc (200 mL), washed with aqueous 1N aq. HCl solution (3×50 mL), and the organic layer was concentrated. The crude product was triturated with EtOAc (50 mL) and filtered, and the filter cake was dried to give compound ZB025_2 hydrochloride salt (7.48 g, 84.2%) as a white solid. This compound was used without further purification.

¹H NMR (400 MHZ, DMSO-d₆) δ ppm 13.24 (br s, 1H). 7.98 (br s, 1H). 7.81 (s, 1H). 6.63 (s, 1H). 4.51 (br t, J=5.40 Hz, 1H). 3.35 (br s, 1H). 3.32 (br s, 1H). 3.27 (s, 7H).

### Step 2:

A solution of compound ZB025_2 (2.00 g, 10.0 mmol) in HCl (5 M, 20.0 mL) was stirred at 25° C. for 3 h. The reaction mixture was filtered, washed with EtOH (10 mL), and concentrated in vacuo to give a compound ZB025_3 (1.00 g, 65.0%) as a white solid. The product was used in the next step without further purification.

### Step 3:

HCl (12.0 M, 8.00 mL) was added to a solution of compound ZB025_3 (1.80 g, 11.8 mmol) in dioxane (8 mL). The reaction mixture was stirred at 100° C. for 3 h. The reaction mixture was poured into saturated aq. NaHCO₃ (15 mL) and extracted with EtOAc (3×10 mL). The combined organic layer was concentrated in vacuo to give compound ZB025_4 (2.05 g, crude) as a yellow solid, which was directly used in the next step without further purification.

### Step 4:

PyBOP (4.62 g, 8.88 mmol) and triethylamine (899 mg, 8.88 mmol) were added to a solution of compound ZB025_4 (1.00 g, 7.40 mmol) in DMF (20.0 mL). The mixture was stirred at 25° C. for 30 min, after which N-Boc piperazine (1.79 g, 9.62 mmol) was added. The reaction mixture was then stirred at 25° C. for 3 h. LC-MS showed that the starting material remained, so the reaction was stirred for an additional 17 h at 80° C. The residue was poured into saturated aq. NaHCO₃ (50 mL) and extracted with EtOAc (3×25 mL). The combined organic layer was concentrated in vacuo. The residue was purified by flash silica gel chromatography (Eluent of 10% THF/petroleum ether) to give compound ZB025_5 (995 mg, 44.3%) as a yellow oil.

### Step 5:

A solution of HCl in dioxane (4 M, 15.0 mL) was added to a solution of compound ZB025_5 (995 mg, 3.28 mmol) in MeOH (5 mL). The mixture was stirred at 25° C. for 1 h. The reaction mixture was concentrated in vacuo to give compound ZB025 (670 mg, 85.2%, HCl salt) as a yellow solid. The product was used without further purification.

¹H NMR (400 MHZ, DMSO-d₆) δ ppm 9.72 (br s, 2H), 8.28 (d, J=5.02 Hz, 1H), 8.14 (d, J=2.51 Hz, 1H), 7.41 (d, J=5.02 Hz, 1H), 7.26 (s, 1H), 4.02 (m, 4H), 3.27 (br s, 4H).

### General Procedure for Preparation of Compound ZB026

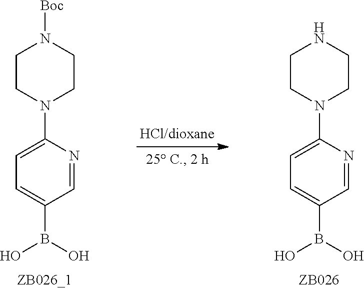 [SMILES: CC(C)(C)OC(=O)N1CCN(c2ccc(B(O)O)cn2)CC1 ; OB(O)c1ccc(N2CCNCC2)nc1]

Compound ZB026_1 (500 mg, 1.63 mmol) was added to a solution of HCl in dioxane (4 M, 6 mL), and the reaction was stirred at 25° C. for 2 h. The reaction mixture was concentrated under reduced pressure to give a residue, which was used in the next step without further purification. Compound ZB026 (430 mg, crude, HCl salt) was obtained as a white solid.

¹H NMR (400 MHz, DMSO-d₆) δ ppm 9.62 (br s, 2H), 8.40 (d, J=1.2 Hz, 1H), 8.24 (br d, J=8.8 Hz, 1H), 7.35 (br d, J=8.4 Hz, 1H), 4.02 (br s, 4H), 3.31-3.23 (m, 1H), 3.27 (br s, 3H).

### General Procedure for Preparation of Compound Z27

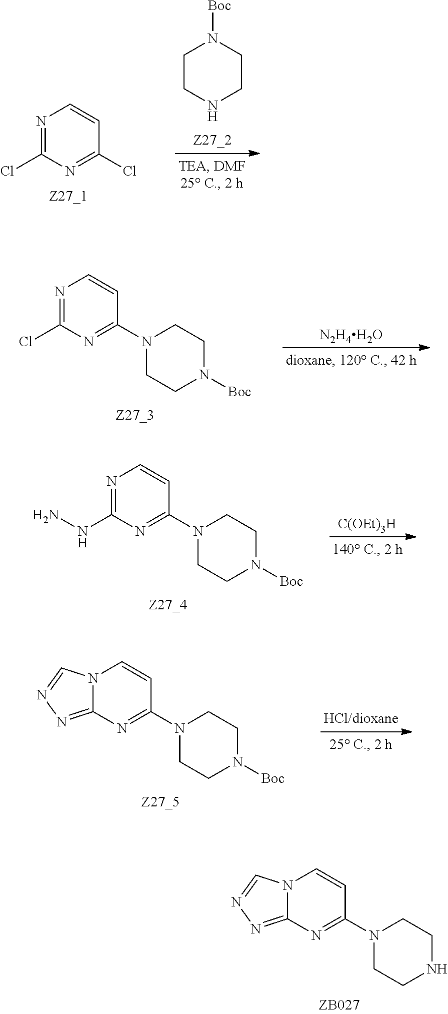

### Step 1:

Triethylamine (4.08 g, 40.2 mmol) was added to a solution of compound Z27_1 (3 g, 20.1 mmol) and compound Z27_2 (3.75 g, 20.1 mmol) in DMF (30 mL). The reaction mixture was stirred at 25° C. for 2 h, after which it was poured into water (100 mL) and stirred for 5 min. The solid was collected by filtration and dried to give compound Z27_3 (4 g, 6.5%) as a yellow solid. The product was used without further purification.

### Step 2:

Hydrazine hydrochloride (1.38 g, 20.1 mmol) was added to a solution of compound Z27_3 (4 g, 13.4 mmol) and triethylamine (4.06 g, 40.1 mmol) in dioxane (40 mL). The reaction mixture was stirred at 120° C. for 18 h. LC-MS showed that starting material remained, so the reaction mixture was stirred at 120° C. for another 24 h. The reaction mixture was cooled and filtered, and the filtrate was concentrated in vacuo to give compound Z27_4 (3 g, 76.1%) as a yellow solid. The product was used without further purification.

### Step 3:

A solution of compound Z27_4 (800 mg, 2.72 mmol) in CH(OEt)₃ (10 mL) was stirred at 140° C. for 2 h. The mixture was cooled and filtered, and the filter cake was dried to give compound Z27_5 (550 mg, 66%) as a yellow solid. The product was used without further purification.

¹H NMR (400 MHZ, DMSO-d₆) δ ppm 8.72 (s, 1H), 8.56 (br d, J=7.70 Hz, 1H), 6.88 (br d, J=7.70 Hz, 1H), 3.72 (br s, 4H), 3.44 (br s, 4H), 1.43 (s, 9H).

### Step 4:

A solution of compound Z27_5 (500 mg, 1.64 mmol) in a solution of HCl in dioxane (4 M, 10 mL, 24.35 eq) was stirred at 25° C. for 2 h. The mixture was concentrated to give compound ZB027 (380 mg, 96%, HCl salt) as a yellow solid. The product was used without further purification.

¹H NMR (400 MHZ, DMSO-d₆) δ ppm 10.01 (br s, 2H), 9.16 (s, 1H), 8.86 (d, J=8.00 Hz, 1H), 7.38 (d, J=7.88 Hz, 1H), 4.15 (br s, 4H), 3.25 (br s, 4H).

### General Procedure for Preparation of Compound ZB028

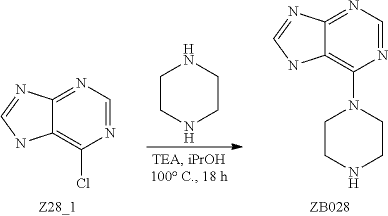 [SMILES: Clc1ncnc2nc[nH]c12 ; C1CNCCN1 ; c1nc(N2CCNCC2)c2[nH]cnc2n1]

A mixture of 6-chloro-7H-purine (500 mg, 3.24 mmol), piperazine (278 mg, 3.24 mmol), and triethylamine (982 mg, 9.71 mmol) in i-PrOH (10 mL) was stirred at 100° C. for 18 h. The precipitate was collected and dried to give compound ZB028 (600 mg, 90.8%) as a yellow solid. The product was used without further purification.

¹H NMR (400 MHZ, DMSO-d₆) δ ppm 8.27 (s, 1H), 8.20 (s, 1H), 4.44 (br s, 4H), 3.19 (br s, 4H).

### General Procedure for Preparation of Compound ZB029

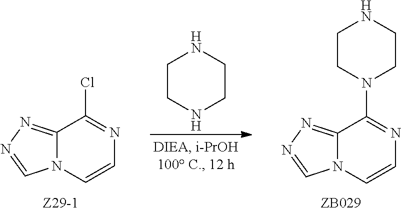 [SMILES: Clc1nccn2cnnc12 ; C1CNCCN1 ; c1cn2cnnc2c(N2CCNCC2)n1]

DIEA (836 mg, 6.47 mmol, 1.13 mL) and piperazine (306 mg, 3.56 mmol) were added to a solution of compound Z29-1 (500 mg, 3.24 mmol) in i-PrOH (1 mL). The reaction mixture was stirred at 100° C. for 12 h, after which the mixture was filtered. The filter cake was dried under reduced pressure to give compound ZB029 (600 mg, crude) as a gray solid which was used in the next step without further purification.

¹H NMR (400 MHZ, DMSO-d₆) δ ppm 9.35-9.30 (m, 1H), 8.00-7.93 (m, 1H), 7.40 (d, J=4.5 Hz, 1H), 4.54-4.35 (m, 4H), 3.31-3.18 (m, 4H), 1.04 (d, J=6.1 Hz, 1H).

### General Procedure for Preparation of Compound ZB037

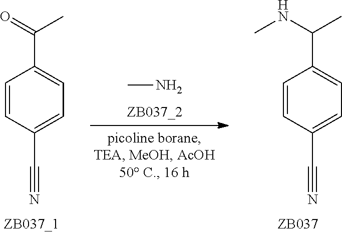

Triethylamine (6.97 g, 68.8 mmol, 9.59 mL), picoline borane (11.0 g, 103 mmol), and compound ZB037_2 (2.79 g, 41.3 mmol, HCl salt) were added to a solution of compound ZB037_1 (5.00 g, 34.4 mmol) in MeOH (50.0 mL) and AcOH (5.00 mL). The reaction mixture was stirred at 50° C. for 16 h, after which it was concentrated under reduced pressure. The residue was purified by flash silica gel chromatography (Eluent of 0˜100% THF/Petroleum ether). Compound ZB037 (1.68 g, 30%) was obtained as a white solid.

¹H NMR (400 MHZ, DMSO-d₆) δ ppm 7.80 (d, J=8.3 Hz, 2H), 7.56 (d, J=8.0 Hz, 2H), 7.42 (br d, J=6.3 Hz, 3H), 3.76 (q, J=6.8 Hz, 1H), 2.13 (s, 3H), 1.27 (d, J=6.8 Hz, 3H).

### General Procedure for Preparation of Compound Z41

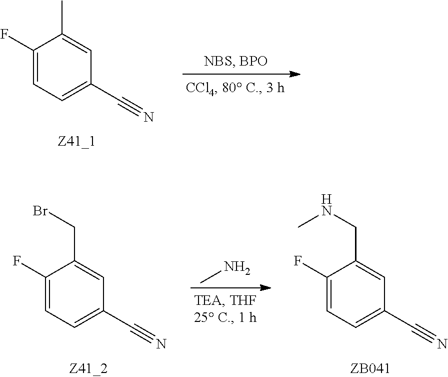

### Step 1:

BPO (5.92 g, 24.4 mmol) and NBS (4.35 g, 24.4 mmol) were added to a solution of compound Z41_1 (3 g, 22.2 mmol) in CCl₄ (50 mL). The mixture was stirred at 80° C. for 3 h. The mixture was cooled to 0° C., and a white solid precipitated. The suspension was filtered, and the filtrate was concentrated. The residue was purified by flash silica gel chromatography (Eluent of 0˜10% Ethyl acetate/Petroleum ether) to give compound Z41_2 (2.5 g, 52.6%) as a white solid.

¹H NMR (400 MHZ, CDCl₃) δ ppm 7.75 (dd, J=6.85, 2.08 Hz, 1H), 7.63 (ddd, J=8.56, 4.71, 2.14 Hz, 1H), 7.19 (t, J=8.93 Hz, 1H), 4.48 (s, 2H).

### Step 2:

MeNH₂·HCl (265 mg, 3.92 mmol) and triethylamine (993 mg, 9.81 mmol) were added to a solution of compound Z41_2 (700 mg, 3.27 mmol) in THF (5 mL). The mixture was stirred at 25° C. for 1 h. The mixture was filtered and concentrated to give compound ZB041 (480 mg, 89.4%) as a yellow solid, which was used without further purification.

### General Procedure for Preparation of Compound ZB042

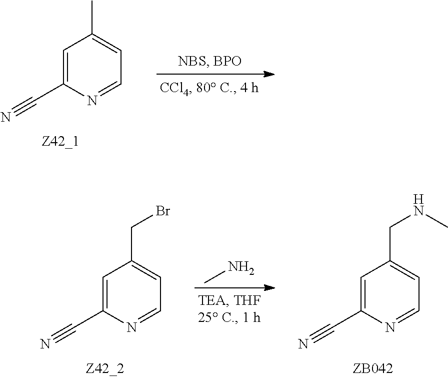

Benzoyl benzenecarboperoxoate (11.3 g, 46.6 mmol) and NBS (8.29 g, 46.6 mmol) were added to a solution of compound Z42_1 (5 g, 42.3 mmol) in CCl₄ (50 mL). The reaction mixture was stirred at 70° C. for 4 h, after which it was filtered and concentrated under reduced pressure. The residue was purified by flash silica gel chromatography (Eluent of 20% Ethyl acetate/Petroleum ether). Compound Z42_2 (2.2 g, 23.7%) was obtained as a brown solid.

¹H NMR (400 MHZ, CDCl₃) δ ppm 8.63 (d, J=5.0 Hz, 1H), 7.66 (d, J=0.9 Hz, 1H), 7.47 (dd, J=1.7, 4.9 Hz, 1H), 4.36 (s, 2H).

Triethylamine (308 mg, 3.05 mmol) and compound Z42_2 (0.6 g, 3.05 mmol) were added to a solution of MeNH₂ (472 mg, 7.00 mmolHCl salt) in THF (5 mL). The reaction mixture was stirred at 25° C. for 1 h, after which it was filtered and concentrated under reduced pressure. The residue was purified by flash silica gel chromatography (Eluent of 80% Ethyl acetate/Petroleum ether). Compound ZB042 (0.18 g, 36.2%) was obtained as a brown solid.

¹H NMR (400 MHZ, CDCl₃) δ ppm 8.66 (d, J=5.0 Hz, 1H), 7.75 (d, J=0.7 Hz, 1H), 7.53 (td, J=0.8, 5.0 Hz, 1H), 3.87 (s, 2H), 2.48 (s, 4H).

### General Procedure for Preparation of Compound ZB044

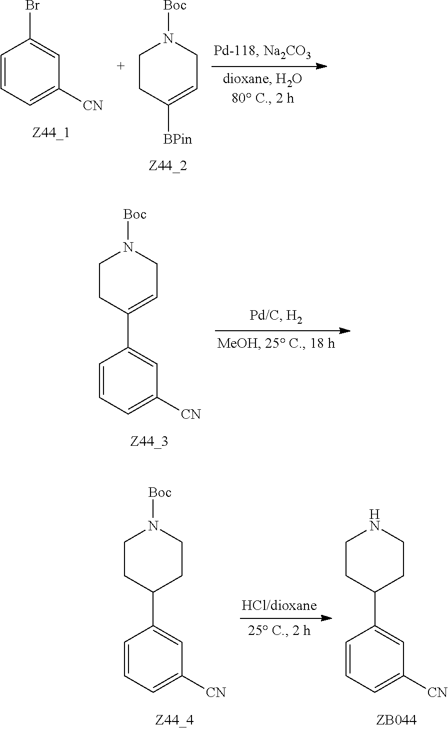

Na₂CO₃ (685 mg, 6.47 mmol) and Pd-118 (105 mg, 161 μmol) were added to a solution of Z44_2 (1 g, 3.23 mmol) and Z44_1 (588 mg, 3.23 mmol) in dioxane (10 mL) and H₂O (3 mL) under N₂. The reaction mixture was stirred at 80° C. for 2 h, after which it was poured into water (20 mL) and extracted with EtOAc (2×30 mL). The combined organic layer was dried with MgSO₄, filtered, and concentrated in vacuo. The residue was purified by flash silica gel chromatography (Eluent of 0˜30% Ethyl acetate/Petroleum ether). Compound Z44_3 (800 mg, 86.9%) was obtained as a yellow oil.

Pd/C (50 mg, 2.81 mmol, 10% wt) was added to a solution of Z44_3 (800 mg, 2.81 mmol) in MeOH (20 mL) under H₂. The reaction mixture was stirred at 25° C. for 18 h, after which it was filtered and concentrated in vacuo. The crude product was used in the next step directly. Compound Z44_4 (670 mg, crude) was obtained as a colorless oil.

¹H NMR (400 MHZ, DMSO-d₆) δ ppm 7.74 (s, 1H), 7.67 (dt, J=7.58, 1.34 Hz, 1H), 7.62 (m, 1H), 7.51 (m, 1H), 4.08 (br d, J=10.64 Hz, 2H), 2.77 (m, 3H), 1.75 (br d, J=12.59 Hz, 2H), 1.53 (m, 2H), 1.42 (s, 9H).

A solution of Z44_4 (400 mg, 1.40 mmol) in a solution of HCl in dioxane (20 mL) was stirred at 25° C. for 2 h. The mixture was concentrated in vacuo, and the crude product was used in the next step directly without further purification. Compound ZB044 (370 mg, crude, HCl salt) was obtained as a white solid.

¹H NMR (400 MHZ, DMSO-d₆) δ ppm 9.37 (br s, 2H), 7.62 (m, 4H), 3.33 (br d, J=12.23 Hz, 2H), 2.97 (m, 3H), 1.95 (m, 4H).

### General Procedure for Preparation of Compound Z45

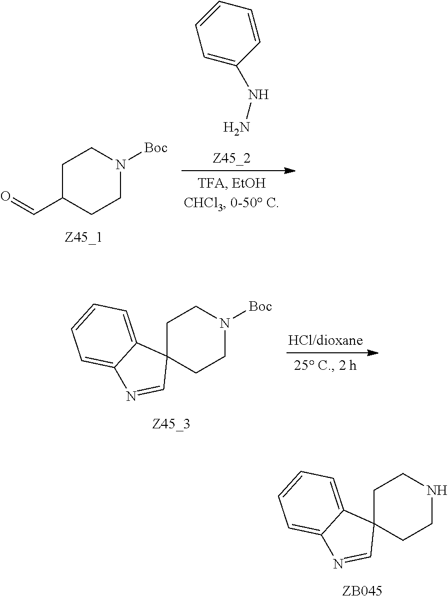 [SMILES: NNc1ccccc1 ; CC(C)(C)OC(=O)N1CCC(C=O)CC1 ; CC(C)(C)OC(=O)N1CCC2(C=Nc3ccccc32)CC1 ; C1=Nc2ccccc2C12CCNCC2]

### Step 1:

A solution of compound Z45_1 (2 g, 9.38 mmol) and compound Z45_2 (1.01 g, 9.38 mmol) in CHCl₃ (100 mL) and EtOH (0.1 mL) was cooled to 0° C., after which TFA (3.21 g, 28.1 mmol) was added dropwise. The mixture was then stirred at 50° C. for 18 h. The mixture was poured into saturated NaHCO₃ (100 mL), and the separated organic layer was washed with brine (50 mL), dried over Na₂SO₄, filtered, and concentrated to give compound Z45_3 (2.66 g, 99.1%) as a brown solid.

¹H NMR (400 MHZ, CDCl₃) δ ppm 8.35 (s, 1H), 7.67 (d, J=7.58 Hz, 1H), 7.35-7.42 (m, 2H), 7.26-7.31 (m, 1H), 3.93-4.08 (m, 2H), 3.49 (ddd, J=13.72, 9.87, 3.55 Hz, 2H), 1.81 (ddd, J=13.69, 9.66, 4.03 Hz, 2H), 1.66 (dt, J=13.66, 4.29 Hz, 2H), 1.51 (s, 9H).

### Step 2:

A solution of compound Z45_3 (800 mg, 2.79 mmol) in HCl/dioxane (4 M, 8.00 mL) was stirred at 25° C. for 2 h. The reaction mixture was concentrated to give compound ZB045 (610 mg, 98.04%, HCl salt) as a yellow solid. The compound was used without further purification.

¹H NMR (400 MHZ, DMSO-d₆) δ ppm 9.46-9.75 (m, 2H), 8.96 (br s, 1H), 7.62 (br d, J=6.78 Hz, 1H), 7.34-7.47 (m, 3H), 3.36-3.45 (m, 4H), 2.21-2.36 (m, 2H), 1.62 (br d, J=14.05 Hz, 2H).

### General Procedure for Preparation of Compound Z50_2

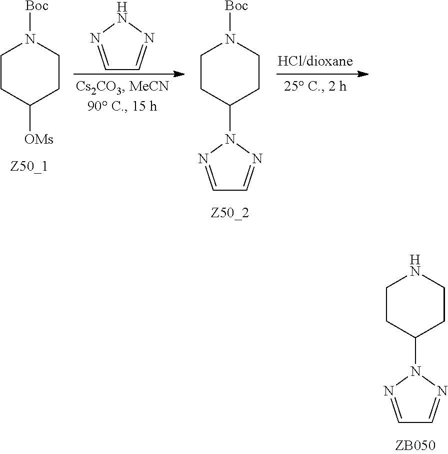

To a solution of compound Z50_1 (1.50 g, 5.37 mmol) and 2H-triazole (407 mg, 5.91 mmol) in MeCN (15 mL) was added Cs₂CO₃ (3.50 g, 10.7 mmol). The reaction mixture was stirred at 90° C. for 15 h, after which it was partitioned between water (200 mL) and ethyl acetate (3×300 mL). The combined organic layer was washed with brine (200 mL), dried over MgSO₄, filtered, and concentrated under reduced pressure. The residue was purified by flash silica gel chromatography (Eluent of 0-45% THF/Petroleum ether). Compound Z50_2 (0.70 g, 49.1%) was obtained as a white solid.

¹H NMR (400 MHZ, DMSO-d₆) δ ppm 7.79 (s, 2H), 4.74 (tt, J=4.1, 11.0 Hz, 1H), 3.97 (br d, J=12.8 Hz, 2H), 3.01 (br s, 2H), 2.16-2.04 (m, 2H), 1.84 (dq, J=4.3, 11.9 Hz, 2H), 1.42 (s, 9H).

To a solution of compound Z50_2 (700 mg, 2.77 mmol) in dioxane (4 mL) was added a solution of HCl in dioxane (4M, 4 mL). The mixture was stirred at 25° C. for 2 h. The reaction mixture was concentrated under reduced pressure to give compound ZB050 (390 mg, crude, HCl salt) as a yellow residue which was used in the next step without further purification.

¹H NMR (400 MHZ, DMSO-d₆) δ ppm 9.62-9.19 (m, 2H), 7.83 (s, 2H), 4.93-4.83 (m, 1H), 3.33 (br d, J=12.9 Hz, 2H), 3.11 (br s, 2H), 2.33-2.19 (m, 4H).

### General Procedure for Preparation of Compound Z54_2

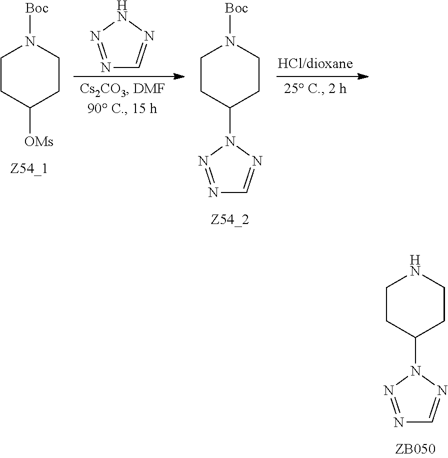

To a solution of 2H-tetrazole (4 M, 4.03 mL) in DMF (15.0 mL) was added Cs₂CO₃ (3.50 g, 10.7 mmol) and compound Z54_1 (1.50 g, 5.37 mmol). The reaction mixture was stirred at 90° C. for 15 h, after which it was partitioned between water (200 mL) and ethyl acetate (3×300 mL). The combined organic layer was washed with brine (200 mL), dried over MgSO₄, filtered, and concentrated under reduced pressure. The residue was purified by flash silica gel chromatography (Eluent of 50% THF/Petroleum ether, gradient). Compound Z54_2 (0.60 g, 41.9%) was obtained as a white solid.

¹H NMR (400 MHZ, CDCl₃) δ ppm 8.53 (s, 1H), 5.04-4.87 (m, 1H), 4.20 (br d, J=13.5 Hz, 2H), 3.07 (ddd, J=3.1, 11.0, 13.8 Hz, 2H), 2.35-2.14 (m, 4H), 1.53-1.48 (m, 9H).

To a solution of HCl in dioxane (4 M, 2.47 mL) was added to a solution of compound Z54_2 (0.50 g, 1.97 mmol) in dioxane (3 mL). The reaction was stirred at 25° C. for 2 h, and a white solid formed. The reaction mixture was concentrated under reduced pressure, and the residue was used in the next step without further purification. Compound ZB054 (0.37 g, 93.9%, HCl salt) was obtained as a white solid.

¹H NMR (400 MHz, DMSO-d₆) δ ppm 9.47-9.10 (m, 2H), 9.04 (s, 1H), 5.31-5.19 (m, 1H), 3.45-3.30 (m, 2H), 3.15 (br d, J=12.3 Hz, 2H), 2.41-2.27 (m, 4H).

### General Procedure for Preparation of Compound Z59_3

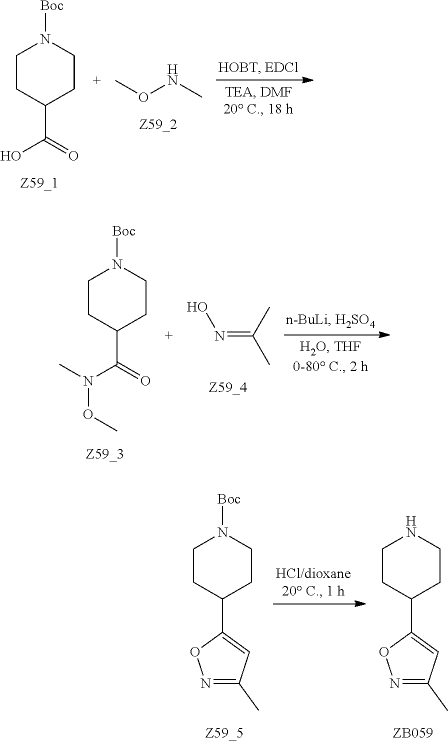

Triethylamine (4.41 g, 43.6 mmol, 6.07 mL) was added to a solution of Z59_1 (5 g, 21.8 mmol) and Z59_2 (2.77 g, 28.3 mmol, HCl salt) in DMF (40 mL). The reaction was allowed to stir for 10 min, after which HOBt (3.83 g, 28.3 mmol) and EDCI (5.43 g, 28.3 mmol) were added. The mixture was stirred at 20° C. for 18 h. The mixture was poured onto water (60 mL) and extracted with EtOAc (3×80 mL). The combined organic layer was washed with brine (3×200 mL), dried with MgSO₄, filtered, and concentrated. The crude product was used in the next step directly without further purification. Compound Z59_3 (6.1 g, crude) was obtained as a colorless oil.

¹H NMR (400 MHz, DMSO-d₆) δ ppm 3.96 (br d, J=11.86 Hz, 2H), 3.69 (s, 3H), 3.33 (br s, 1H), 3.10 (s, 3H), 2.83 (m, 4H), 1.64 (br d, J=11.00 Hz, 2H), 1.40 (s, 9H).

A solution of Z59_4 (1.07 g, 14.6 mmol, 1.19 mL) in THF (26 mL) was cooled to 0° C. and then a solution of n-BuLi (2.5 M, 11.7 mL) was added dropwise over 5 min. The reaction was stirred for 30 min, and then a solution of Z59_3 (2 g, 7.34 mmol) in THF (20 mL) was added dropwise to the reaction mixture over 20 min. The reaction was stirred for 30 min, after which it was poured into a solution of H₂SO₄ (3.60 g, 36.7 mmol) in THF and water (v:v=4:1, 6 mL) and refluxed for 1 h. The mixture was carefully neutralized with NaHCO₃, and then water was added to dissolve the salts. The mixture was extracted with EtOAc (3×80 mL), and the combined organic layer was washed with brine, dried with MgSO4, and concentrated in vacuo. The residue was purified by flash silica gel chromatography (Eluent of 0˜25% THF/petroleum ether). Compound Z59_5 (1.6 g, 81.80%) was obtained as a colorless oil.

¹H NMR (400 MHZ, DMSO-d₆) δ ppm 6.14 (s, 1H), 3.95 (br d, J=12.96 Hz, 2H), 2.95 (m, 3H), 2.19 (s, 3H), 1.91 (m, 2H), 1.45 (m, 2H), 1.40 (s, 9H).

A solution of Z59_5 (1.1 g, 4.13 mmol) in a solution of HCl in dioxane (20 mL) was stirred at 20° C. for 1 h. The mixture was concentrated in vacuo to give a white solid. The crude product was used in the next step directly without further purification. Compound ZB059 (700 mg, HCl salt) was obtained as a white solid.

¹H NMR (400 MHZ, DMSO-d₆) δ ppm 9.24 (m, 1H), 9.26 (m, 2H), 6.19 (s, 1H), 3.27 (br d, J=12.84 Hz, 2H), 3.13 (tt, J=11.34, 3.58 Hz, 1H), 2.99 (m, 2H), 2.20 (s, 3H), 2.08 (m, 2H), 1.85 (m, 2H).

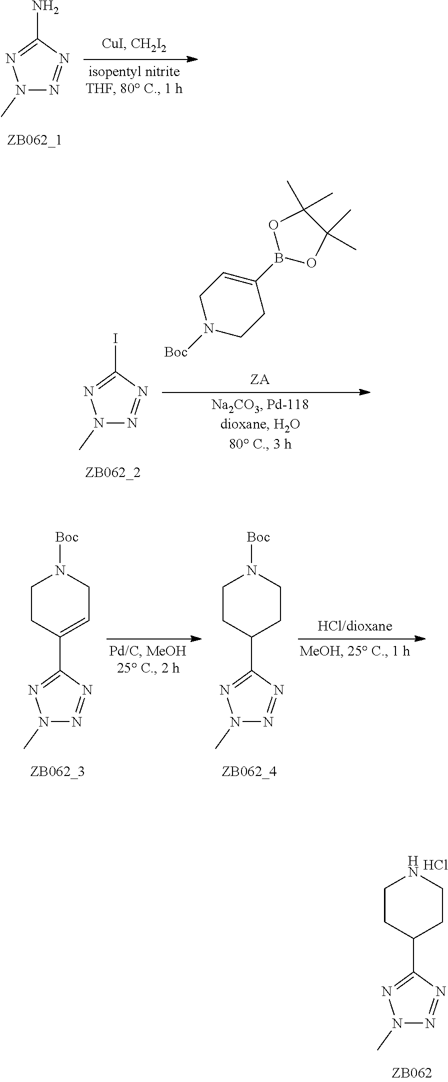

### General Procedure for Preparation of Compound ZB062

### Step 1:

Isopentyl nitrite (5.11 g, 43.6 mmol) was added to a solution of compound ZB062_1 (2.00 g, 12.1 mmol), CH₂I₂ (16.2 g, 60.6 mmol), and CuI (2.31 g, 12.1 mmol) in THF (21 mL). The reaction mixture was stirred at 80° C. for 1 h, after which it was poured into saturated NaCl (20 mL) and extracted with EtOAc (3×20 mL). The combined organic layer was concentrated in vacuo. The residue was purified by flash silica gel chromatography (Eluent of 9% Ethyl acetate/Petroleum ether) to give compound ZB062_2 (2.12 g, 83.4%) as a yellow solid.

### Step 2:

To a solution of compound ZB062_2 (500 mg, 2.38 mmol) and compound ZA (957 mg, 3.10 mmol) in dioxane (10 mL) and H₂O (2 mL) was added Na₂CO₃ (505 mg, 4.76 mmol) and Pd-118 (77.6 mg, 119 μmol) under N₂. The reaction mixture was stirred at 80° C. for 3 h under N₂. The residue was poured into water (15 mL), extracted with EtOAc (3×10 mL), and the combined organic layer was concentrated in vacuo. The residue was purified by flash silica gel chromatography (Eluent of 10% Ethyl acetate/Petroleum ether) to give compound ZB062_3 (400 mg, 63.3%) as a yellow solid.

### Step 3:

To a solution of compound ZB062_3 (400 mg, 1.51 mmol) in MeOH (6.00 mL) was added Pd/C (1.51 mmol, 1.00 mL, 10% wt) under H₂. The reaction mixture was stirred at 25° C. for 2 h under H₂. The mixture was filtered and concentrated in vacuo to give compound ZB062_4 (400 mg, crude) as a white solid, which was used directly in the next step without further purification.

### Step 4:

To a solution of compound ZB062_4 (400 mg, 1.50 mmol) in MeOH (2 mL) was added a solution of HCl in dioxane (4 M, 7 mL). The reaction mixture was stirred at 25° C. for 1 h. The mixture was concentrated in vacuo to give compound ZB062 (295 mg, 96.8%, HCl salt) as a white solid, which was used directly in the next step without further purification.

¹H NMR (400 MHZ, DMSO-d₆) δ ppm 9.30 (m, 2H), 4.32 (s, 2H), 4.31 (m, 1H), 3.27 (m, 3H), 3.04 (m, 2H), 2.14 (br dd, J=13.88, 2.75 Hz, 2H), 1.97 (m, 2H).

### General Procedure for Preparation of Compound ZB063

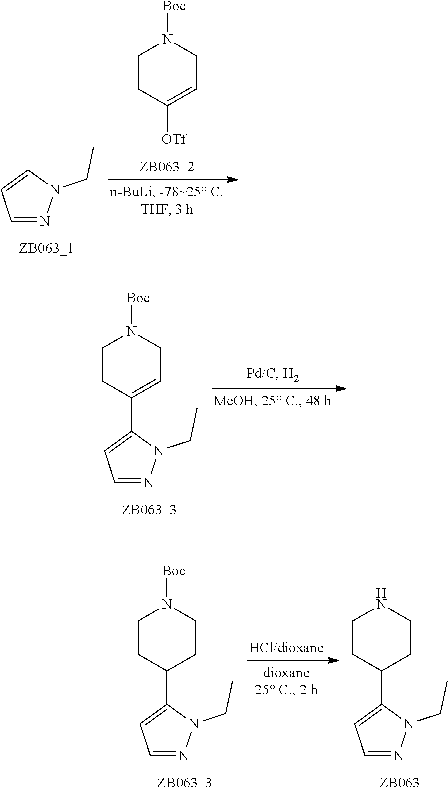

### Step 1:

To a solution of compound ZB063_1 (1.00 g, 10.4 mmol) in THF (10.0 mL) was added n-BuLi (2.5 M, 4.58 mL) dropwise at −78° C. under N₂. The reaction mixture was stirred at −78° C. for 1 h. Then a solution of compound ZB063_2 (3.45 g, 10.4 mmol) in THF (10.0 mL) was added dropwise at −78° C. After the addition, the reaction mixture was allowed to warm to 25° C. slowly. The mixture was stirred at 25° C. for 2 h. The reaction mixture was poured into saturated aq. NH₄Cl (30.0 mL) and extracted with EtOAc (30 mL). The combined organic layer was washed with brine (30 mL), dried over Na₂SO₄, filtered, and concentrated. The residue was purified by flash silica gel chromatography (Eluent of 0˜50% Ethyl acetate/Petroleum ether). Compound ZB063_3 (700 mg, 24.2%) was obtained as a brown oil.

¹H NMR (400 MHZ, DMSO-d₆) δ ppm 7.43-7.37 (m, 1H), 6.20 (d, J=1.8 Hz, 1H), 5.88 (br s, 1H), 4.16-4.06 (m, 2H), 4.00 (br s, 2H), 3.57-3.44 (m, 2H), 2.39-2.23 (m, 2H), 1.43 (s, 9H), 1.35-1.29 (m, 3H).

### Step 2:

To a solution of compound ZB063_3 (700 mg, 2.52 mmol) in MeOH (3 mL) was added Pd/C (500 mg, 2.52 mmol). The reaction mixture was stirred at 25° C. for 48 h. The reaction mixture was filtered and concentrated under reduced pressure. The crude product was used in the next step without further purification. Compound ZB063_4 (650 mg, crude) was obtained as a yellow solid.

### Step 3:

To a solution of compound ZB063_4 (600 mg, 2.15 mmol) in dioxane (5 mL) was added a solution of HCl in dioxane (4 M, 5 mL). The reaction mixture was stirred at 25° C. for 2 h. The reaction mixture was concentrated under reduced pressure. The crude product was used in the next step without further purification. Compound ZB063 (296 mg, HCl salt) was obtained as a yellow oil.

¹H NMR (400 MHZ, DMSO-d₆) δ ppm 9.68-9.20 (m, 2H), 7.57-7.46 (m, 1H), 6.24-6.09 (m, 1H), 5.41-5.24 (m, 2H), 4.16 (q, J=7.4 Hz, 2H), 3.28 (br d, J=12.0 Hz, 2H), 1.97-1.77 (m, 4H), 1.33 (t, J=7.2 Hz, 3H).

### General Procedure for Preparation of Compound Z64_2

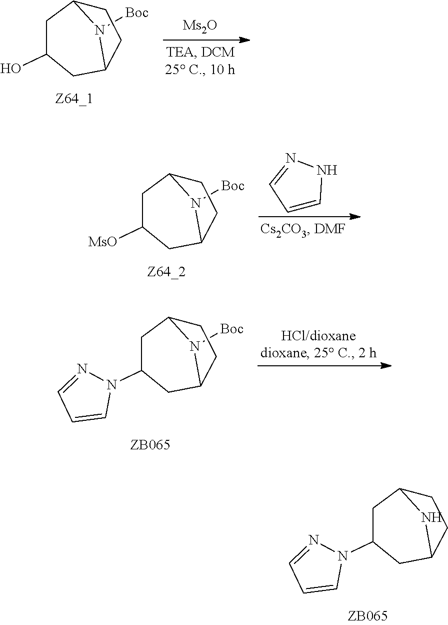

To a solution of compound Z64_1 (4.00 g, 17.6 mmol) in dichloromethane (40 mL) was added triethylamine (3.56 g, 35.2 mmol, 4.90 mL) and methyl sulfonyl methane sulfonate (6.13 g, 35.2 mmol). The mixture was stirred at 25° C. for 10 h. The reaction was partitioned between water (400 mL) and dichloromethane (600 mL). The organic layer was washed with brine (100 mL), dried over MgSO₄, filtered, and concentrated under reduced pressure. The product was used in the next step without further purification. Compound Z⁶⁴_2, which is a mixture of cis and trans stereoisomers, (5.00 g, 93.0%) was obtained as a yellow oil.

¹H NMR (400 MHZ, DMSO-d₆) δ ppm 5.03-4.90 (m, 1H), 4.10 (br s, 1H), 3.24-3.13 (m, 3H), 2.06 (br s, 2H), 1.97-1.79 (m, 4H), 1.77-1.68 (m, 1H), 1.77-1.56 (m, 2H), 1.41 (d, J=6.1 Hz, 9H).

To a solution of compound Z64_2 (0.60 g, 1.96 mmol) in DMF (6 mL) was added Cs₂CO₃ (638.61 mg, 1.96 mmol) and 1H-pyrazole (200 mg, 2.94 mmol). The mixture was stirred at 90° C. for 15 h. The reaction was partitioned between water (200 mL) and ethyl acetate (3×300 mL). The combined organic layer was washed with brine (200 mL), dried over MgSO₄, filtered, and concentrated under reduced pressure. The residue was purified by flash silica gel chromatography (Eluent of 50% THF/Petroleum ether). Compound Z65_1, which is a mixture of cis and trans stereoisomers, (0.45 g, 78.6%) was obtained as a white solid.

¹H NMR (400 MHZ, DMSO-d₆) δ ppm 7.98 (d, J=2.3 Hz, 1H), 7.47 (d, J=1.4 Hz, 1H), 6.29 (t, J=2.0 Hz, 1H), 4.47-4.28 (m, 1H), 4.09 (br s, 2H), 2.56 (br d, J=14.1 Hz, 2H), 2.33-2.17 (m, 2H), 1.75-1.63 (m, 2H), 1.44 (s, 11H).

To a solution of compound Z65_1 (0.45 g, 1.62 mmol) in dioxane (2 mL) was added a solution of HCl in dioxane (2 mL). The mixture was stirred at 25° C. for 2 h. The reaction mixture was concentrated under reduced pressure to give compound ZB065, which is a mixture of cis and trans stereoisomers, (295 mg, HCl salt), a white solid which was used in the next step without further purification.

¹H NMR (400 MHZ, DMSO-d₆) δ ppm 9.23 (br s, 2H), 8.05 (d, J=2.1 Hz, 1H), 7.50 (d, J=1.4 Hz, 1H), 6.35 (t, J=1.9 Hz, 1H), 4.49-4.45 (m, 2H), 3.98 (br s, 2H), 3.39 (s, 1H), 2.87 (br d, J=15.8 Hz, 2H), 1.86-1.75 (m, 2H), 1.60-1.52 (m, 2H), 1.39-1.14 (m, 3H), 0.90-0.71 (m, 1H).

### General Procedure for Preparation of Compound Z64-3

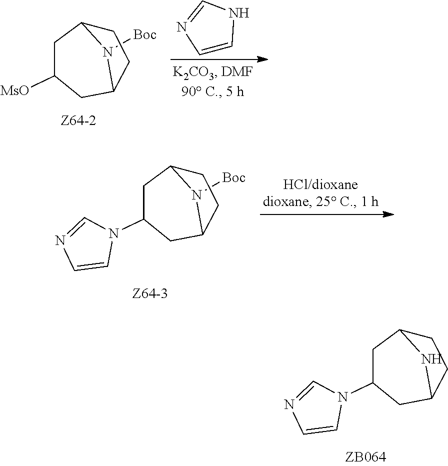

To a solution of compound Z64_2 (800 mg, 2.62 mmol) in DMF (8.00 mL) was added Cs₂CO₃ (2.56 g, 7.86 mmol) and imidazole (713 mg, 10.5 mmol). The mixture was stirred at 90° C. for 5 h. The reaction mixture was poured into water (50 mL), and the product was extracted with ethyl acetate (50 mL). The combined organic layer was dried with Na₂SO₄, filtered, and concentrated under reduced pressure. The residue was purified by flash silica gel chromatography (Eluent of 0˜30% Ethyl acetate/Petroleum ether) to give compound Z64-3, which is a mixture of cis and trans stereoisomers, (290 mg, 37.9%) as a white solid.

¹H NMR (400 MHZ, DMSO-d₆) δ ppm 7.85 (s, 1H), 7.36 (s, 1H), 6.91 (s, 1H), 4.20-4.08 (m, 3H), 2.41-2.29 (m, 2H), 2.11 (br dd, J=4.3, 14.3 Hz, 2H), 1.80 (br s, 2H), 1.53 (br d, J=7.3 Hz, 2H), 1.43 (s, 9H). To a solution of compound Z64-3 (290 mg, 1.05 mmol) in dioxane (2.00 mL) was added a solution of HCl in dioxane (4M, 2 mL). The mixture was stirred at 25° C. for 1 h. The reaction mixture was concentrated under reduced pressure to give compound ZB064, which is a mixture of cis and trans stereoisomers, (260 mg, crude, HCl salt) as a white residue which was used in the next step without further purification.

¹H NMR (400 MHz, DMSO-d₆) δ ppm 9.60-9.34 (m, 3H), 8.09 (s, 1H), 7.82 (s, 1H), 4.67 (br s, 1H), 4.03 (br s, 2H), 2.70 (br s, 4H), 1.98-1.86 (m, 2H), 1.45 (br d, J=8.5 Hz, 2H).

### General Procedure for Preparation of Compound ZB066

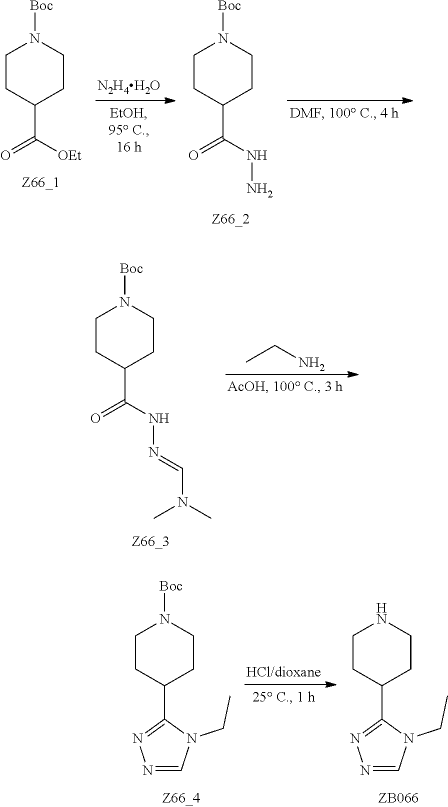

### Step 1:

To a solution of ZB066_1 (6.95 g, 27.0 mmol) in EtOH (100 mL) was added N₂H₄·H₂O (6.90 g, 135 mmol). The mixture was stirred at 95° C. for 16 h. The solvent was removed under reduced pressure to give compound ZB066_2 (6.50 g, crude) as a white solid which was used in the next step without further purification.

### Step 2:

To a solution of ZB066_2 (1.70 g, 6.99 mmol) in DMF (20.0 mL) was added 1,1-dimethoxy-N,N-dimethyl-methanamine (832 mg, 6.99 mmol). The mixture was stirred at 100° C. for 4 h. The reaction mixture was concentrated under reduced pressure to give compound ZB066_3 (1.8 g, crude) as a colorless residue which was used in the next step without further purification.

### Step 3:

To a solution of compound Z66_3 (2 g, 6.70 mmol) in AcOH (15 mL) was added ethanamine (3.02 g, 67.03 mmol) dropwise at 0° C. The mixture was stirred at 100° C. for 3 h. The mixture was concentrated to give a yellow residue. The residue was dissolved in EtOAc (100 mL), washed with brine (50 mL), dried over Na₂SO₄, filtered, and concentrated to give a crude product. The crude product was used directly in the next step without further purification. Compound Z66_4 (1.8 g, 67.1%) was obtained as a yellow solid.

¹H NMR (400 MHZ, CDCl₃) δ ppm 8.17 (s, 1H), 4.02-4.29 (m, 3H), 3.96 (q, J=7.34 Hz, 2H), 3.21-3.36 (m, 1H), 1.79-1.93 (m, 4H), 1.67-1.79 (m, 2H), 1.43-1.50 (m, 12H).

### Step 4:

A solution of compound Z66_4 (1.8 g, 4.49 mmol,) in a solution of HCl in dioxane (4 M, 30 mL) was stirred at 25° C. for 1 h. The mixture was concentrated to give a white solid. The solid was directly used in the next step without further purification. Compound ZB066 (1.3 g, 93.4%, HCl salt) was obtained as a white solid.

### General Procedure for Preparation of Compound Z67_1 and Z68_1

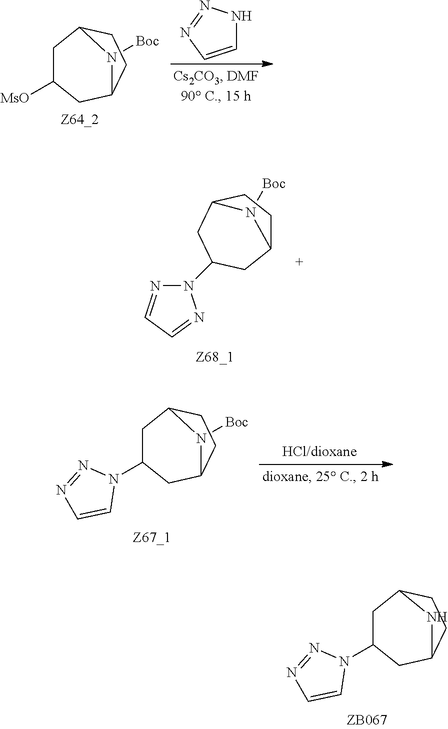

To a solution of compound Z64_2 (3.00 g, 9.82 mmol) in DMF (30 mL) was added Cs₂CO₃ (3.20 g, 9.82 mmol) and 2H-triazole (1.02 g, 14.7 mmol). The mixture was stirred at 100° C. for 2 h. The reaction was partitioned between water (300 mL) and ethyl acetate (3×300 mL). The combined organic layer was washed with brine (200 mL), dried over MgSO₄, filtered, and concentrated under reduced pressure. The residue was purified by flash silica gel chromatography (Eluent of 10-60% THF/Petroleum ether, gradient). Compound Z68_1 (0.65 g, 22.6%) was obtained as a white solid.

Compound Z68_1 (0.35 g, 1.19 mmol, 12.2%, 95% purity) was obtained as a white solid.

¹H NMR (400 MHZ, CDCl₃) δ ppm 7.60 (s, 2H), 5.03 (tt, J=5.7, 11.7 Hz, 1H), 4.40 (br s, 2H), 2.43-2.19 (m, 2H), 2.15-1.99 (m, 4H), 1.87-1.75 (m, 2H), 1.51 (s, 9H).

Compound Z67_1 (0.90 g, 3.07 mmol, 31.3%) was obtained as a white solid.

¹H NMR (400 MHZ, CDCl₃) δ ppm 7.79 (s, 1H), 7.72 (s, 1H), 4.63 (quin, J=5.0 Hz, 1H), 4.42 (br s, 1H), 4.33 (br s, 2H), 2.65-2.46 (m, 5H), 2.14 (td, J=2.9, 10.6 Hz, 2H), 1.89 (dq, J=3.0, 5.3 Hz, 2H), 1.60-1.54 (m, 2H), 1.51 (s, 13H).

To a solution of compound Z67_1 (0.90 g, 3.23 mmol) in dioxane (10 mL) was added a solution of HCl in dioxane (4 M, 4.85 mL). The mixture was stirred at 25° C. for 2 h. White solid was formed. The reaction mixture was concentrated under reduced pressure. The residue was used in the next step without further purification. Compound ZB067, which is a mixture of cis and trans stereoisomers, (0.69 g, 99.4%, HCl salt) was obtained as a white solid.

¹H NMR (400 MHZ, DMSO-d₆) δ ppm 10.15-9.41 (m, 4H), 8.57-8.16 (m, 1H), 7.92-7.65 (m, 1H), 5.21-4.66 (m, 1H), 4.20-3.93 (m, 2H), 2.98-2.79 (m, 1H), 2.68 (ddd, J=3.3, 6.2, 16.2 Hz, 1H), 2.27-2.16 (m, 1H), 2.06 (s, 2H), 1.87-1.76 (m, 1H), 1.49-1.37 (m, 1H).

### General Procedure for Preparation of Compound ZB068

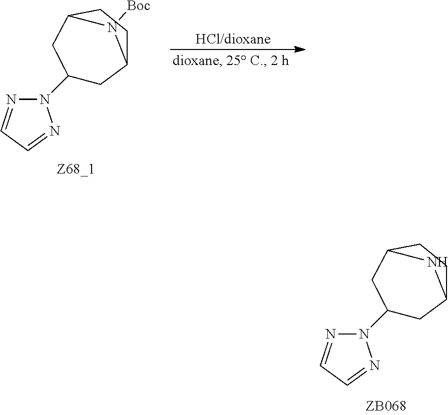 [SMILES: CC(C)(C)OC(=O)N1C2CCC1CC(n1nccn1)C2 ; c1cnn(C2CC3CCC(C2)N3)n1]

To a solution of compound Z68_1 (0.65 g, 2.34 mmol) in dioxane (10 mL) was added a solution of HCl in dioxane (4 M, 3.50 mL). The mixture was stirred at 25° C. for 2 h. The reaction mixture was concentrated under reduced pressure. The residue was used in the next step without further purification. Compound ZB068, which is a mixture of cis and trans stereoisomers, (0.50 g, 99.7%, HCl salt) was obtained as a white solid.

¹H NMR (400 MHZ, DMSO-d₆) δ ppm 9.41 (br s, 2H), 8.01-7.80 (m, 2H), 4.72 (t, J=6.1 Hz, 1H), 4.02 (br s, 2H), 3.07 (br d, J=15.8 Hz, 2H), 2.78-2.56 (m, 2H), 1.87-1.68 (m, 2H), 1.42-1.19 (m, 2H).

### General Procedure for Preparation of Compound Z69_1

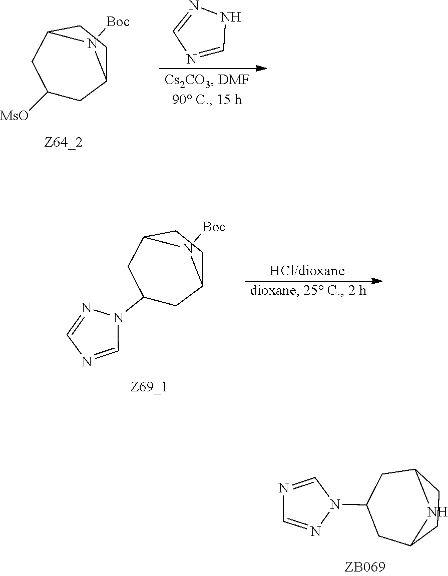 [SMILES: CC(C)(C)OC(=O)N1C2CCC1CC(OS(C)(=O)=O)C2 ; c1nc[nH]n1 ; CC(C)(C)OC(=O)N1C2CCC1CC(n1cncn1)C2 ; c1ncn(C2CC3CCC(C2)N3)n1]

To a solution of compound Z64_2 (1.50 g, 4.91 mmol) in DMF (15 mL) was added Cs₂CO₃ (2.40 g, 7.37 mmol) and 1H-1,2,4-triazole (1.02 g, 14.7 mmol). The mixture was stirred at 100° C. for 2 h. The reaction was partitioned between water (200 mL) and ethyl acetate (3×300 mL). The combined organic layer was washed with brine (200 mL), dried over MgSO₄, filtered, and concentrated under reduced pressure. The residue was purified by flash silica gel chromatography (Eluent of 50% THF/Petroleum ether). Compound Z69_1, which is a mixture of cis and trans stereoisomers, (0.50 g, 34.7%) was obtained as a white solid.

¹H NMR (400 MHZ, CDCl₃) δ ppm 8.36 (s, 1H), 7.92 (s, 1H), 4.39 (quin, J=5.2 Hz, 1H), 4.25 (br s, 2H), 2.39 (br s, 4H), 1.91-1.79 (m, 2H), 1.47 (br d, J=7.6 Hz, 2H), 1.42 (s, 9H).

To a solution of compound Z69_1 (0.50 g, 1.80 mmol) in dioxane (4 mL) was added a solution of HCl in dioxane (4 M, 2.69 mL). The mixture was stirred at 25° C. for 10 h. The reaction mixture was concentrated under reduced pressure. The residue was used in the next step without further purification. Compound ZB069, which is a mixture of cis and trans stereoisomers, (0.38 g, 98.5%, HCl salt) was obtained as a white solid.

¹H NMR (400 MHZ, DMSO-d₆) δ ppm 9.41 (br s, 2H), 9.12 (s, 1H), 8.21 (s, 1H), 4.62 (br t, J=6.3 Hz, 1H), 4.02-3.99 (m, 2H), 2.85-2.75 (m, 2H), 2.66-2.57 (m, 2H), 1.88-1.77 (m, 2H), 1.55-1.43 (m, 2H).

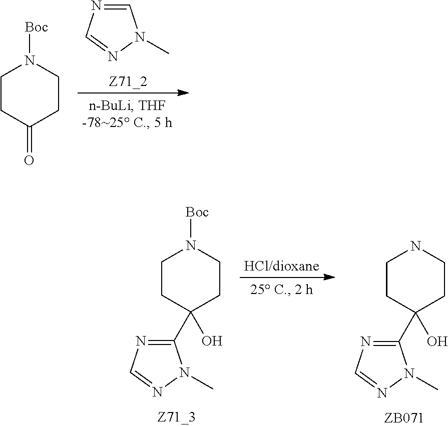

### General Procedure for Preparation of Compound Z71

To a solution of compound Z71_2 (1 g, 12.0 mmol) in THF (20 mL) was added n-BuLi (2.5 M, 5.30 mL) dropwise at −78° C. under N₂. The mixture was stirred at −78° C. for 0.5 h. Then a solution of compound Z71_1 (2.40 g, 12.0 mmol) in THF (10 mL) was added dropwise at −78° C. After the addition, the mixture was allowed to slowly warm to 25° C. The mixture was stirred at 25° C. for 1.5 h. The mixture was poured into saturated aq. NaH₄Cl (30 mL) and extracted with EtOAc (30 mL). The organic layer was washed with brine (30 mL), dried over Na₂SO₄, filtered, and concentrated to give a yellow residue. The residue was purified by flash silica gel chromatography (Eluent of 0˜60% Ethyl acetate/Petroleum ether) to give compound Z71_3 (3.2 g, 94.2%) as a yellow solid.

¹H NMR (400 MHZ, DMSO-d₆) δ ppm 7.77 (s, 1H), 5.76 (s, 1H), 3.98 (s, 3H), 3.69 (br d, J=13.05 Hz, 2H), 3.22 (br d, J=2.01 Hz, 2H), 1.86-1.98 (m, 2H), 1.78-1.85 (m, 2H), 1.40 (s, 9H).

### Step 2:

A solution of compound Z71_3 (700 mg, 2.48 mmol) in a solution of HCl in dioxane (4 M, 10 mL) was stirred at 25° C. for 2 h. The mixture was concentrated to give compound ZB071 (540 mg, 99.6%, HCl salt) as a yellow solid, which was used without further purification.

¹H NMR (400 MHZ, DMSO-d₆) δ ppm 8.95-9.37 (m, 2H), 7.87 (s, 1H), 3.99 (s, 3H), 3.16 (br s, 3H), 3.10-3.22 (m, 1H), 2.20-2.34 (m, 2H), 2.05 (br d, J=13.76 Hz, 1H), 1.95-2.12 (m, 1H).

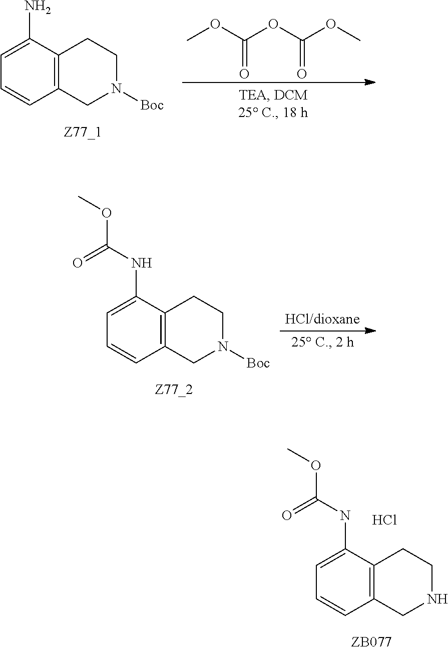

### General Procedure for Preparation of Compound Z77

### Step 1:

To a solution of compound Z77_1 (1 g, 4.03 mmol) in THF (15 mL) was added triethylamine (1.22 g, 12.1 mmol) and methoxycarbonyl methyl carbonate (591 mg, 4.43 mmol, 475 μL). The mixture was stirred at 25° C. for 18 h. The mixture was diluted with EtOAc (30 mL), washed with saturated aq. NaHCO₃ (30 mL) and brine (30 mL), dried over Na₂SO₄, filtered, and concentrated. The residue was purified by flash silica gel chromatography (Eluent of 0˜50% Ethyl acetate/Petroleum ether) to give compound Z77_2 (330 mg, 26.7%) as a white solid.

¹H NMR (400 MHZ, DMSO-d₆) δ ppm 8.87 (s, 1H), 7.06-7.26 (m, 2H), 6.98 (br d, J=7.50 Hz, 1H), 4.48 (br s, 2H), 3.64 (s, 3H), 3.51 (br t, J=5.88 Hz, 2H), 2.64 (br t, J=5.82 Hz, 2H), 1.42 (s, 9H)

### General Procedure for Preparation of Compound Z72

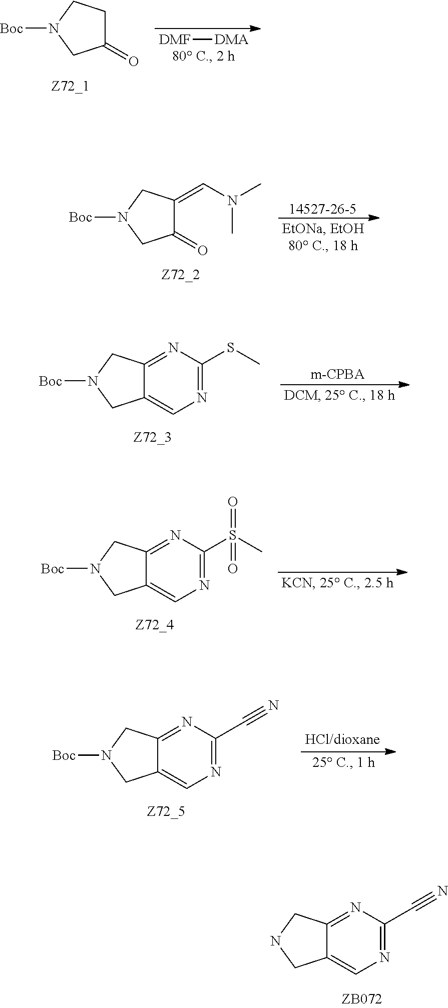

### Step 1:

A solution of compound Z72_1 (5 g, 26.99 mmol) in DMF-DMA (17.9 g, 150 mmol) was stirred at 80° C. for 2 h. The mixture was then concentrated to give a yellow solid, which was used directly in the next step without further purification. Compound Z72_2 (6.49 g, crude) was obtained as a yellow solid.

### Step 2:

To a solution of 2-methylisothiourea; sulfuric acid (6.95 g, 24.9 mmol) in EtOH (200 mL) was added NaOEt (1.70 g, 24.9 mmol) at 0° C. under N₂. The mixture was stirred at 0° C. for 1 h. Then compound Z72_2 (6 g, 24.97 mmol) was added in batches. The resulting mixture was stirred at 80° C. for 18 h. The mixture was concentrated, and the residue was partitioned between EtOAc (200 mL) and brine (200 mL). The organic layer was separated and concentrated to give a yellow residue. The residue was purified by flash silica gel chromatography (Eluent of 0˜30% Ethyl acetate/Petroleum ether) to give compound Z72_3 (1.1 g, 16.5%) as a yellow solid.

### Step 3:

To a solution of compound Z72_3 (1 g, 3.74 mmol) in dichloromethane (30 mL) was added m-CPBA (1.52 g, 7.48 mmol, 85% purity). The mixture was stirred at 25° C. for 16 h. The reaction mixture was washed with saturated aqueous Na₂SO₃ (50 mL) and brine (30 mL), dried over Na₂SO₄, filtered, and concentrated under reduced pressure. The residue was purified by flash silica gel chromatography (Eluent of 0˜40% Ethyl acetate/Petroleum ether) to give compound Z72_4 (900 mg, 80.4%) as a yellow solid.

### Step 4:

To a solution of compound Z72_4 (700 mg, 2.34 mmol) in DMF (7 mL) was added KCN (228 mg, 3.51 mmol) at 25° C. The mixture was stirred at 25° C. for 2.5 h. The reaction mixture was diluted with H₂O (30 mL) and extracted with EtOAc (2×30 mL). The combined organic layer was washed with brine (20 mL), dried over Na₂SO₄, filtered, and concentrated under reduced pressure. The residue was purified by flash silica gel chromatography (Eluent of 0˜30% Ethyl acetate/Petroleum ether) to give compound Z72_5 (440 mg, 76%), which was obtained as a white solid.

### Step 5:

A solution of compound Z72_5 (500 mg, 1.42 mmol) in a solution of HCl in dioxane (4 M, 10 mL) was stirred at 25° C. for 20 min. The mixture was concentrated to give a white solid. The solid was directly used in the next step without further purification. Compound ZB072 (250 mg, 96.3%, HCl salt) was obtained as a white solid.

### 3.2 Experimental Details for Final Compound Synthesis Using the Procedures in 3.1

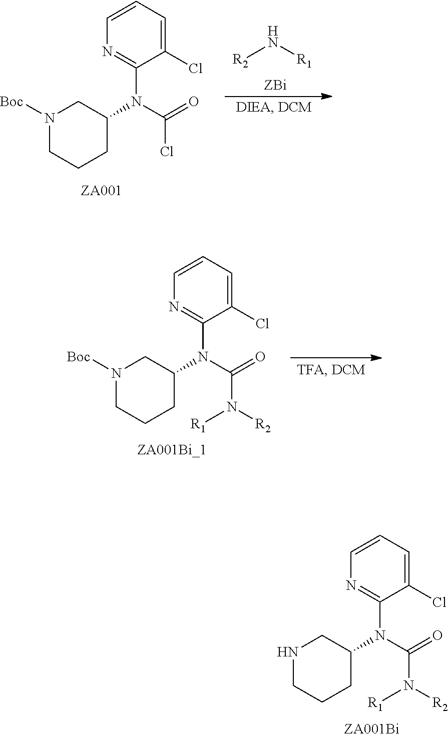

### Procedure Z1

### Step 1

To a solution of ZBi (60 μmol, 1.2 eq) and ZA001 (18.7 mg, 50 μmol, 1.0 eq) in dichloromethane (100 μL) was added DIEA (150 μmol, 3.0 eq). The reaction vials were capped and shaken at 30° C. for 16 h. The reaction mixture was concentrated under reduced pressure to remove solvent. The residue was diluted with H₂O (1.0 mL) and extracted with dichloromethane (3×1.5 mL). The combined organic layer was concentrated under reduced pressure to give ZA001Bi_1, which was used in the next step directly.

### Step 2

To a solution of ZA001Bi_1 (50 μmol, 1.0 eq) was added a solution (1.0 mL) of dichloromethane and TFA (v/v=5/1). The reaction mixture was shaken at 30° C. for 2 h, after which it was concentrated under reduced pressure to give a residue, which was then purified by preparative HPLC to obtain the final product.

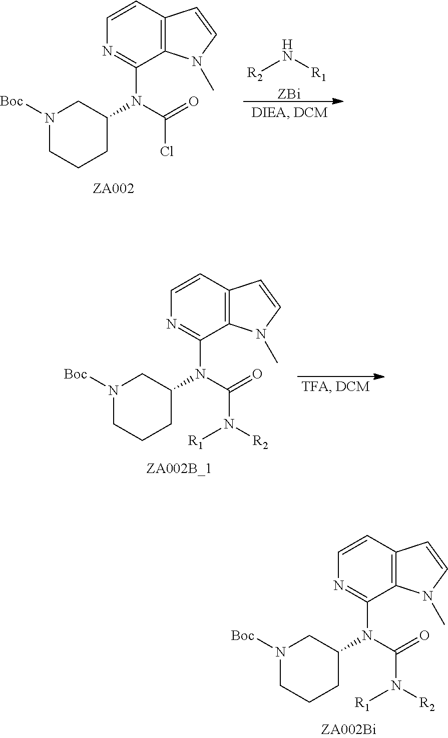

### Procedure Z2

### Step 1

To a solution of ZBi (60 μmol) and ZA002 (19.6 mg, 50 μmol) in dichloromethane (100 μL) was added DIEA (150 μmol). The reaction vials were capped and shaken at 50° C. for 16 h. The reaction mixture was concentrated under reduced pressure to remove solvent. The residue was diluted with H₂O (1.0 mL) and extracted with dichloromethane (3×1.5 mL). The combined organic layer was concentrated under reduced pressure to give a residue ZA002Bi_1, which was used in the next step directly.

### Step 2

To a solution of ZA002Bi_1 (50 μmol, 1.0 eq) was added a solution (1.0 mL) of dichloromethane and TFA (v/v=5/1). The reaction mixture was shaken at 30° C. for 2 h, after which it was concentrated under reduced pressure to give a residue, which was then purified by preparative HPLC to obtain the final product.

### 4.1 Experimental Details for Intermediate Synthesis

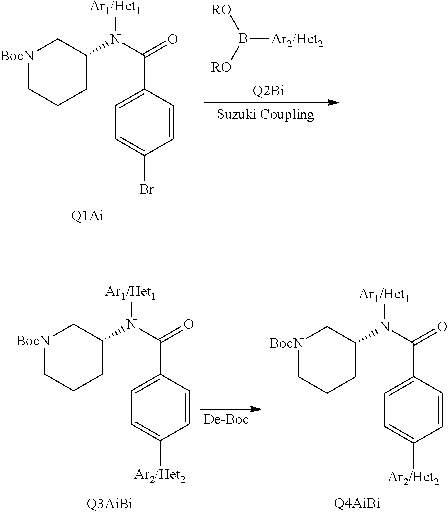

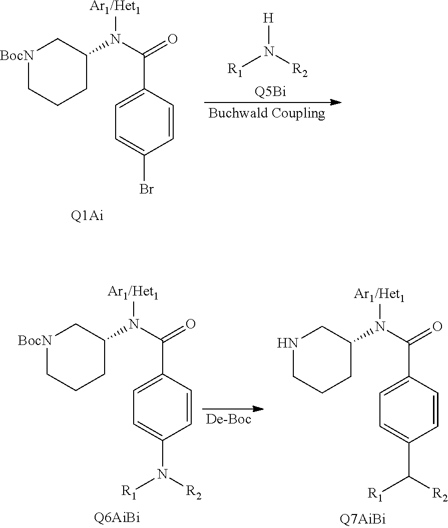

### Procedures for Step 1

### Condition Q1.1

To a solution of Q2Bi (150 μmol, 1.5 eq) and Q1Ai (100 μmol, 1.0 eq) in dioxane (1.0 mL) was added 200 μL of Cs₂CO₃ (1.5 M in H₂O, 300 μmol, 3.0 eq) and XPhos Pd G3 (10 μmol, 0.1 eq) under N₂. The mixture was shaken at 80° C. for 16 h. The reaction mixture was concentrated in vacuo, and the residue was diluted with H₂O (1.0 mL) and extracted with ethyl acetate (3×1.5 mL). The combined organic layer was combined and concentrated in vacuo to give crude Q3AiBi, which was used in the next step directly.

### Condition Q1.2

To a solution of Q2Bi (150 μmol, 1.5 eq) and Q1Ai (100 μmol, 1.0 eq) in dioxane (1.0 mL) was added 200 μL of Cs₂CO₃ (1.5 M in H₂O, 300 μmol, 3.0 eq) and XPhos Pd G3 (10 μmol, 0.1 eq) under N₂. The mixture was shaken at 80° C. for 16 h. The reaction mixture was concentrated in vacuo, and the residue was diluted with H₂O (1.0 mL) and extracted with ethyl acetate (3×1.5 mL). The combined organic layer was combined and concentrated in vacuo to give crude Q3AiBi, which was used in the next step directly.

### Condition Q1.3

To a solution of Q2Bi (150 μmol, 1.5 eq) and Q1Ai (100 μmol, 1.0 eq) in dioxane (1.0 mL) was added 100 μL of K₃PO₄ (1.0 M in H₂O, 300 μmol, 2.0 eq) and Pd 118 (10 μmol, 0.1 eq) under N₂. The mixture was shaken at 80° C. for 4 h. The reaction mixture was concentrated in vacuo, and the residue was diluted with H₂O (1.0 mL) and extracted with ethyl acetate (3×1.5 mL). The combined organic layer was combined and concentrated in vacuo to give crude Q3AiBi, which was used in the next step directly.

### Condition Q1.4

To a solution of Q2Bi (150 μmol, 1.5 eq) and Q1Ai (100 μmol, 1.0 eq) in dioxane (1.0 mL) was added Pd(PPh₃)₂Cl₂ (10 μmol, 0.1 eq) under N₂. The mixture was shaken at 90° C. for 16 h. The reaction mixture was concentrated in vacuo, and the residue was diluted with H₂O (1.0 mL) and extracted with ethyl acetate (3×1.5 mL). The combined organic layer was combined and concentrated in vacuo to give crude Q3AiBi, which was used in the next step directly.

### Condition Q1.5

To a solution of Q5Bi (150 μmol, 1.5 eq) and Q1Ai (100 μmol, 1.0 eq) in dioxane (1.0 mL) was added Cs₂CO₃ (300 μmol, 3.0 eq,) and RuPhos Pd G4 (10 μmol, 0.1 eq) in dichloroethane (30 μL) under N₂. The mixture was shaken at 100° C. for 16 h. The reaction mixture was concentrated in vacuo, and the residue was diluted with H₂O (1.0 mL) and extracted with ethyl acetate (3×1.5 mL). The combined organic layer was combined and concentrated in vacuo to give crude Q6AiBi, which was used in the next step directly.

### Condition Q1.6

To a solution of Q5Bi (150 μmol, 1.5 eq) and Q1Ai (100 μmol, 1.0 eq) in dioxane (1.0 mL) was added t-BuONa (300 μmol, 3.0 eq,) and RuPhos Pd G4 (10 μmol, 0.1 eq) in dichloroethane (30 μL) under N₂. The mixture was shaken at 100° C. for 16 h. The reaction mixture was concentrated in vacuo, and the residue was diluted with H₂O (1.0 mL) and extracted with ethyl acetate (3×1.5 mL). The combined organic layer was combined and concentrated in vacuo to give crude Q6AiBi, which was used in the next step directly.

### Condition Q1.7

To a solution of Q5Bi (150 μmol, 1.5 eq) and Q1Ai (100 μmol, 1.0 eq) in dioxane (1.0 mL) was added Cs₂CO₃ (300 μmol, 3.0 eq,) and BrettPhos Pd G3 (10 μmol, 0.1 eq) in dichloroethane (30 μL) under N₂. The mixture was shaken at 120° C. for 16 h. The reaction mixture was concentrated in vacuo, and the residue was diluted with H₂O (1.0 mL) and extracted with ethyl acetate (3×1.5 mL). The combined organic layer was combined and concentrated in vacuo to give crude Q6AiBi, which was used in the next step directly.

### Condition Q1.8

To a solution of Q5Bi (150 μmol, 1.5 eq) and Q1Ai (100 μmol, 1.0 eq) in dioxane (1.0 mL) was added Cs₂CO₃ (300 μmol, 3.0 eq) and RuPhos Pd G4 (10 μmol, 0.1 eq) in dichloroethane (30 μL) under N₂. The mixture was shaken at 150° C. under N₂ for 2 h. The reaction mixture was concentrated in vacuo, and the residue was diluted with H₂O (1.0 mL) and extracted with ethyl acetate (3×1.5 mL). The combined organic layer was combined and concentrated in vacuo to give crude Q6AiBi, which was used in the next step directly.

### Condition Q1.9

To a solution of Q5Bi (150 μmol, 1.5 eq) and Q1Ai (100 μmol, 1.0 eq) in dioxane (1.0 mL) was added K₂CO₃ (300 μmol, 3.0 eq) and BrettPhos Pd G3 (10 μmol, 0.1 eq) in dichloroethane (30 μL) under N₂. The mixture was shaken at 120° C. for 16 h. The reaction mixture was concentrated in vacuo, and the residue was diluted with H₂O (1.0 mL) and extracted with ethyl acetate (3×1.5 mL). The combined organic layer was combined and concentrated in vacuo to give crude Q6AiBi, which was used in the next step directly.

### Procedures for Step 2

### Condition Q2.1

1.0 mL of a solution of dichloromethane and TFA (v/v=3/1) was added to Q3AiBi or Q6AiBi (80 μmol, 1.0 eq). The reaction mixture was shaken at 30° C. for 2 h. The reaction mixture was concentrated in vacuo, and the residue was diluted with 2.0 mL of MeOH and adjusted to a pH of 8˜9 with NH₃·H₂O. The residue was then purified by preparative HPLC to obtain the final product.

### Condition Q2.2

To a solution of Q3AiBi or Q6AiBi (80 μmol, 1.0 eq) in 1 mL of dichloromethane was added 0.5 mL of HCl-dioxane (4.0 M). The reaction mixture was shaken at 30° C. for 2 h. The reaction mixture was concentrated in vacuo, and the residue was purified by preparative HPLC to obtain the final product.

### Condition Q2.3

To a solution of Q3AiBi or Q6AiBi (80 μmol, 1.0 eq) was added CF₃CH₂OH (1 mL) and HBF₄ (30 μL, 50% of purity). The reaction mixture was shaken at 30° C. for 2 h. The reaction mixture was concentrated in vacuo, and the residue was purified by preparative. HPLC to obtain the final product.

Table 4 below provides a list of compounds made using the procedures describe above. The Ex No. is the Example Number for each exemplified compound.

TABLE 4

| | | | Procedure | |
|---|---|---|---|---|
| EX | | | for | |
| No. | Name | Structure | Synthesis | MS data^{(M + H+)} |
| 1 | (R)-N-(3-chloropyridin-2- yl)-4-(5-methyl-3H- [1,2,3]triazolo[4,5- b]pyridin-3-yl)-N- (piperidin-3-yl)benzamide | 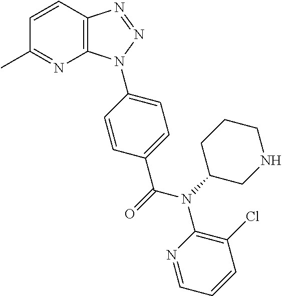 [SMILES: Cc1ccc2nnn(-c3ccc(C(=O)N(c4ncccc4Cl)[C@@H]4CCCNC4)cc3)c2n1] | W1 | 448.2 |
| 2 | (R)-N-(isoquinolin-1-yl)- N-(piperidin-3-yl)-4- (pyrazolo[1,5-a]pyrimidin- 3-yl)benzamide | 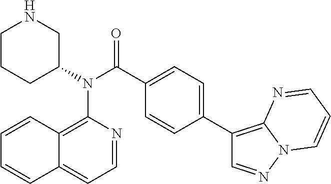 [SMILES: O=C(c1ccc(-c2cnn3cccnc23)cc1)N(c1nccc2ccccc12)[C@@H]1CCCNC1] | W1 | 448.9 |
| 3 | (R)-N-(1-methyl-1H- pyrrolo[2,3-c]pyridin-7- yl)-N-(piperidin-3-yl)-4- (pyrazolo[1,5-a]pyrimidin- 3-yl)benzamide | 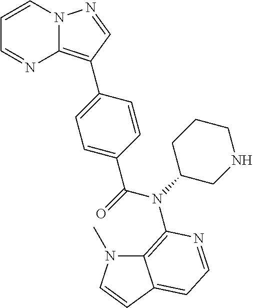 [SMILES: Cn1ccc2ccnc(N(C(=O)c3ccc(-c4cnn5cccnc45)cc3)[C@@H]3CCCNC3)c21] | W1 | 451.9 |
| 4 | (R)-4-(3-cyanophenyl)-N- (1-methyl-1H-pyrrolo[2,3- c]pyridin-7-yl)-N- (piperidin-3-yl)piperidine- 1-carboxamide | 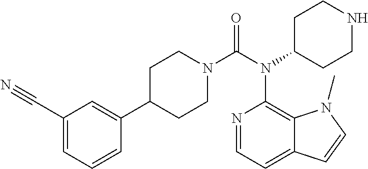 | Z2 | 443.0 |
| 5 | (R)-N-(1-methyl-1H- pyrrolo[2,3-c]pyridin-7- yl)-4-phenyl-N-(piperidin- 3-yl)piperidine-1- carboxamide | 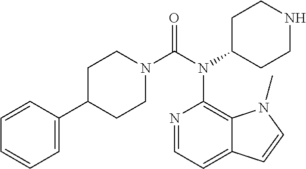 | Z2 | 418.2 |
| 6 | (R)-4-(3H- [1,2,3]triazolo[4,5- b]pyridin-3-yl)-N- (isoquinolin-1-yl)-N- (piperidin-3-yl)benzamide | 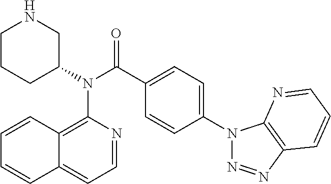 [SMILES: O=C(c1ccc(-n2nnc3cccnc32)cc1)N(c1nccc2ccccc12)[C@@H]1CCCNC1] | W1 | 450.4 |
| 7 | (R)-N-(3-chloropyridin-2- yl)-N-(piperidin-3- yl)spiro[indole-3,4′- piperidine]-1′-carboxamide | 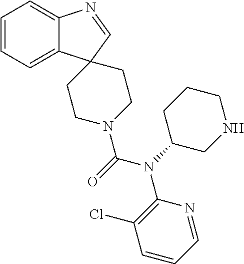 [SMILES: O=C(N1CCC2(C=Nc3ccccc32)CC1)N(c1ncccc1Cl)[C@@H]1CCCNC1] | Z1 | 424.0 |
| 8 | (R)-1-(3-chloropyridin-2- yl)-3-(4-cyanobenzyl)-3- isopropyl-1-(piperidin-3- yl)urea | 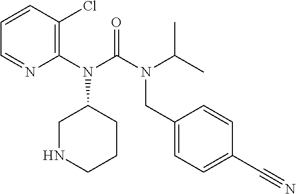 [SMILES: CC(C)N(Cc1ccc(C#N)cc1)C(=O)N(c1ncccc1Cl)[C@@H]1CCCNC1] | Z1 | 412.3 |
| 9 | (R)-N-(3-chloropyridin-2- yl)-N-(piperidin-3-yl)-4- (7H-purin-6-yl)piperazine- 1-carboxamide | 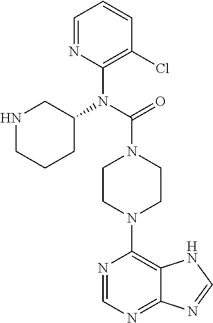 [SMILES: O=C(N1CCN(c2ncnc3nc[nH]c23)CC1)N(c1ncccc1Cl)[C@@H]1CCCNC1] | Z1 | 442.3 |
| 10 | (R)-4-(1-methyl-1H- pyrazol-5-yl)-N-(1-methyl- 1H-pyrrolo[2,3-c]pyridin- 7-yl)-N-(piperidin-3- yl)piperidine-1- carboxamide | 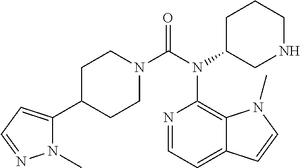 [SMILES: Cn1nccc1C1CCN(C(=O)N(c2nccc3ccn(C)c23)[C@@H]2CCCNC2)CC1] | Z2 | 422.3 |
| 11 | (R)-4-(1-methyl-1H- pyrazol-4-yl)-N-(1-methyl- 1H-pyrrolo[2,3-c]pyridin- 7-yl)-N-(piperidin-3- yl)piperidine-1- carboxamide | 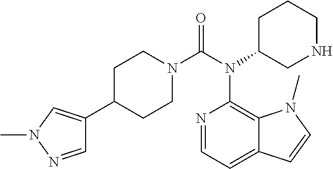 [SMILES: Cn1cc(C2CCN(C(=O)N(c3nccc4ccn(C)c34)[C@@H]3CCCNC3)CC2)cn1] | Z2 | 422.3 |
| 12 | (R)-N-(1-methyl-1H- pyrrolo[2,3-c]pyridin-7- yl)-N-(piperidin-3-yl)-4- (2H-tetrazol-2- yl)piperidine-1- carboxamide | 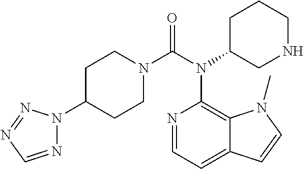 [SMILES: Cn1ccc2ccnc(N(C(=O)N3CCC(n4ncnn4)CC3)[C@@H]3CCCNC3)c21] | Z2 | 410.0 |
| 13 | (R)-N-(1-methyl-1H- pyrrolo[2,3-c]pyridin-7- yl)-N-(piperidin-3-yl)-4- (4H-1,2,4-triazol-4- yl)piperidine-1- carboxamide | 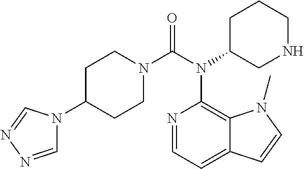 [SMILES: Cn1ccc2ccnc(N(C(=O)N3CCC(n4cnnc4)CC3)[C@@H]3CCCNC3)c21] | Z2 | 409.4 |
| 14 | (R)-N-(1-methyl-1H- pyrrolo[2,3-c]pyridin-7- yl)-N-(piperidin-3-yl)-4- (1H-1,2,3-triazol-1- yl)piperidine-1- carboxamide | 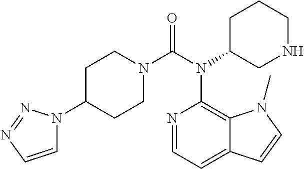 [SMILES: Cn1ccc2ccnc(N(C(=O)N3CCC(n4ccnn4)CC3)[C@@H]3CCCNC3)c21] | Z2 | 409.4 |
| 15 | (R)-N-(1-methyl-1H- pyrrolo[2,3-c]pyridin-7- yl)-N-(piperidin-3-yl)-4- (2H-1,2,3-triazol-2- yl)piperidine-1- carboxamide | 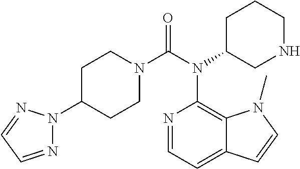 [SMILES: Cn1ccc2ccnc(N(C(=O)N3CCC(n4nccn4)CC3)[C@@H]3CCCNC3)c21] | Z2 | 409.3 |
| 16 | (R)-4-cyano-N-(1-methyl- 1H-pyrrolo[2,3-c]pyridin- 7-yl)-4-phenyl-N- (piperidin-3-yl)piperidine- 1-carboxamide | 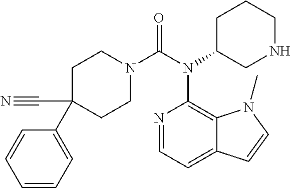 [SMILES: Cn1ccc2ccnc(N(C(=O)N3CCC(C#N)(c4ccccc4)CC3)[C@@H]3CCCNC3)c21] | Z2 | 443.3 |
| 17 | (R)-4-(2-cyanophenyl)-N- (1-methyl-1H-pyrrolo[2,3- c]pyridin-7-yl)-N- (piperidin-3-yl)piperidine- 1-carboxamide | 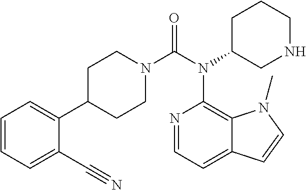 [SMILES: Cn1ccc2ccnc(N(C(=O)N3CCC(c4ccccc4C#N)CC3)[C@@H]3CCCNC3)c21] | Z2 | 443.4 |
| 18 | (R)-1-(5-cyano-2- fluorobenzyl)-1-methyl-3- (1-methyl-1H-pyrrolo[2,3- c]pyridin-7-yl)-3- (piperidin-3-yl)urea | 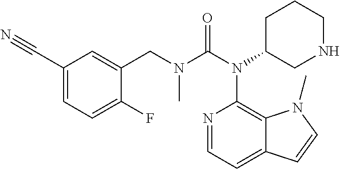 [SMILES: CN(Cc1cc(C#N)ccc1F)C(=O)N(c1nccc2ccn(C)c12)[C@@H]1CCCNC1] | Z2 | 421.3 |
| 19 | (R)-1-(3-cyanobenzyl)-1- methyl-3-(1-methyl-1H- pyrrolo[2,3-c]pyridin-7- yl)-3-(piperidin-3-yl)urea | 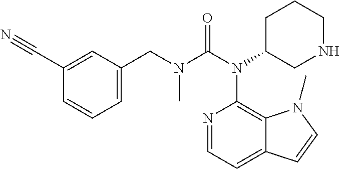 [SMILES: CN(Cc1cccc(C#N)c1)C(=O)N(c1nccc2ccn(C)c12)[C@@H]1CCCNC1] | Z2 | 403.4 |
| 20 | (R)-1-(4-cyanobenzyl)-1- isopropyl-3-(1-methyl-1H- pyrrolo[2,3-c]pyridin-7- yl)-3-(piperidin-3-yl)urea | 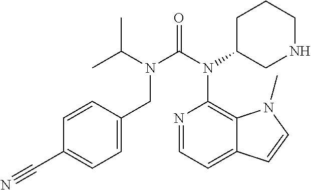 [SMILES: CC(C)N(Cc1ccc(C#N)cc1)C(=O)N(c1nccc2ccn(C)c12)[C@@H]1CCCNC1] | Z2 | 431.4 |
| 21 | (R)-5-cyano-N-(1-methyl- 1H-pyrrolo[2,3-c]pyridin- 7-yl)-N-(piperidin-3- yl)isoindoline-2- carboxamide | 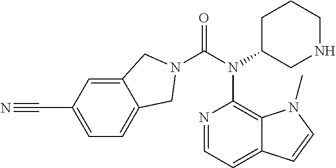 [SMILES: Cn1ccc2ccnc(N(C(=O)N3Cc4ccc(C#N)cc4C3)[C@@H]3CCCNC3)c21] | Z2 | 401.3 |
| 22 | (R)-N-(1-methyl-1H- pyrrolo[2,3-c]pyridin-7- yl)-N-(piperidin-3-yl)-4- (7H-purin-6-yl)piperazine- 1-carboxamide | 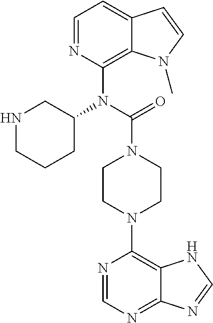 [SMILES: Cn1ccc2ccnc(N(C(=O)N3CCN(c4ncnc5nc[nH]c45)CC3)[C@@H]3CCCNC3)c21] | Z2 | 461.3 |
| 23 | (R)-N-(1-methyl-1H- pyrrolo[2,3-c]pyridin-7- yl)-N-(piperidin-3-yl)-4- (1H-pyrrolo[2,3- d]pyrimidin-4- yl)piperazine-1- carboxamide | 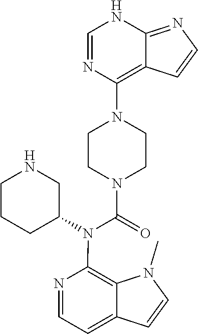 [SMILES: Cn1ccc2ccnc(N(C(=O)N3CCN(c4nc[nH]c5nccc4-5)CC3)[C@@H]3CCCNC3)c21] | Z2 | 460.3 |
| 24 | (R)-4-(4-cyanopyrimidin- 2-yl)-N-(1-methyl-1H- pyrrolo[2,3-c]pyridin-7- yl)-N-(piperidin-3- yl)piperazine-1- carboxamide | 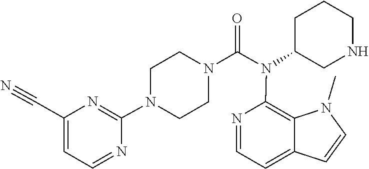 [SMILES: Cn1ccc2ccnc(N(C(=O)N3CCN(c4nccc(C#N)n4)CC3)[C@@H]3CCCNC3)c21] | Z2 | 446.3 |
| 25 | (R)-4-(6-chloropyridin-2- yl)-N-(1-methyl-1H- pyrrolo[2,3-c]pyridin-7- yl)-N-(piperidin-3- yl)piperazine-1- carboxamide | 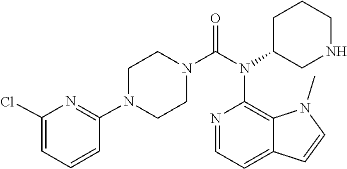 [SMILES: Cn1ccc2ccnc(N(C(=O)N3CCN(c4cccc(Cl)n4)CC3)[C@@H]3CCCNC3)c21] | Z2 | 454.3 |
| 26 | (R)-4-(4-chloropyridin-2- yl)-N-(1-methyl-1H- pyrrolo[2,3-c]pyridin-7- yl)-N-(piperidin-3- yl)piperazine-1- carboxamide | 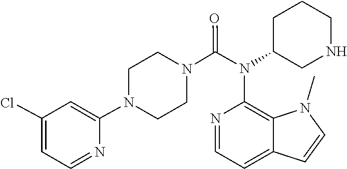 [SMILES: Cn1ccc2ccnc(N(C(=O)N3CCN(c4cc(Cl)ccn4)CC3)[C@@H]3CCCNC3)c21] | Z2 | 454.3 |
| 27 | (R)-N-(1-methyl-1H- pyrrolo[2,3-c]pyridin-7- yl)-N-(piperidin-3-yl)-4- (pyridin-2-yl)piperazine-1- carboxamide | 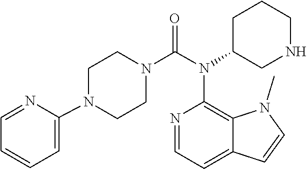 [SMILES: Cn1ccc2ccnc(N(C(=O)N3CCN(c4ccccn4)CC3)[C@@H]3CCCNC3)c21] | Z2 | 420.4 |
| 28 | (R)-4′-cyano-N-(1-methyl- 1H-pyrrolo[3,2-c]pyridin- 6-yl)-N-(piperidin-3-yl)- [1,1′-biphenyl]-4- carboxamide | 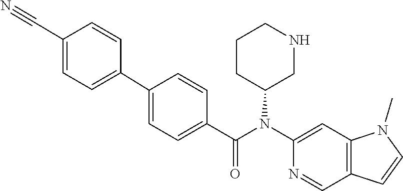 [SMILES: Cn1ccc2cnc(N(C(=O)c3ccc(-c4ccc(C#N)cc4)cc3)[C@@H]3CCCNC3)cc21] | W1 | 436.3 |
| 29 | (R)-4-(3H- [1,2,3]triazolo[4,5- b]pyridin-3-yl)-2-fluoro-N- (1-methyl-1H-pyrrolo[3,2- c]pyridin-6-yl)-N- (piperidin-3-yl)benzamide | 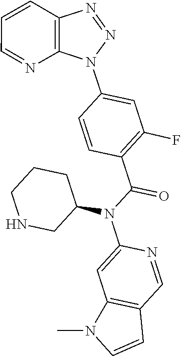 [SMILES: Cn1ccc2cnc(N(C(=O)c3ccc(-n4nnc5cccnc54)cc3F)[C@@H]3CCCNC3)cc21] | W1 | 471.3 |
| 30 | (R)-4′-cyano-N-(1-methyl- 1H-pyrrolo[2,3-c]pyridin- 5-yl)-N-(piperidin-3-yl)- [1,1′-biphenyl]-4- carboxamide | 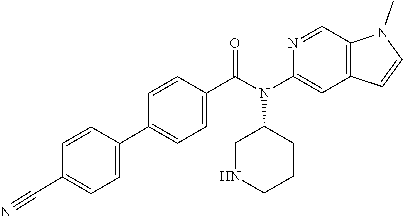 [SMILES: Cn1ccc2cc(N(C(=O)c3ccc(-c4ccc(C#N)cc4)cc3)[C@@H]3CCCNC3)ncc21] | W1 | 436.3 |
| 31 | (R)-4-(3H- [1,2,3]triazolo[4,5- b]pyridin-3-yl)-N-(1- methyl-1H-pyrrolo[2,3- c]pyridin-5-yl)-N- (piperidin-3-yl)benzamide | 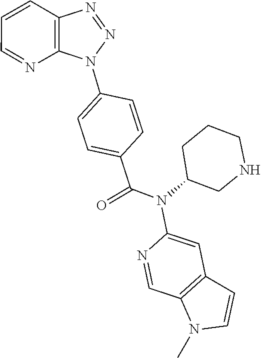 [SMILES: Cn1ccc2cc(N(C(=O)c3ccc(-n4nnc5cccnc54)cc3)[C@@H]3CCCNC3)ncc21] | W1 | 453.3 |
| 32 | (R)-4-(3H- [1,2,3]triazolo[4,5- b]pyridin-3-yl)-2-fluoro-N- (1-methyl-1H-pyrrolo[2,3- c]pyridin-5-yl)-N- (piperidin-3-yl)benzamide | 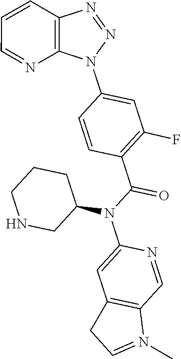 | W1 | 471.3 |
| 33 | (R)-4-(3H- [1,2,3]triazolo[4,5- b]pyridin-3-yl)-2-methyl- N-(1-methyl-1H- pyrrolo[2,3-c]pyridin-5- yl)-N-(piperidin-3- yl)benzamide | 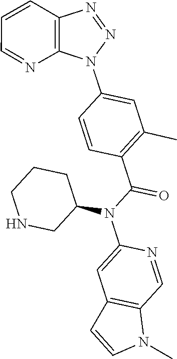 [SMILES: Cc1cc(-n2nnc3cccnc32)ccc1C(=O)N(c1cc2ccn(C)c2cn1)[C@@H]1CCCNC1] | W1 | 467.3 |
| 34 | (R)-4-(5-methyl-1H- pyrazol-1-yl)-N-(1-methyl- 1H-pyrrolo[2,3-c]pyridin- 5-yl)-N-(piperidin-3- yl)benzamide | 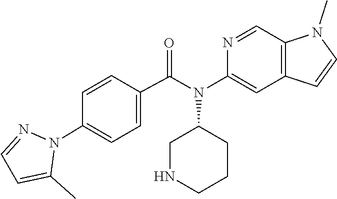 [SMILES: Cc1ccnn1-c1ccc(C(=O)N(c2cc3ccn(C)c3cn2)[C@@H]2CCCNC2)cc1] | W1 | 415.3 |
| 35 | (R)-4-(3H- [1,2,3]triazolo[4,5- b]pyridin-3-yl)-2-ethyl-N- (piperidin-3-yl)-N- (quinolin-2-yl)benzamide | 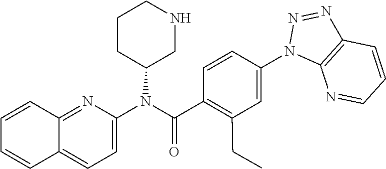 [SMILES: CCc1cc(-n2nnc3cccnc32)ccc1C(=O)N(c1ccc2ccccc2n1)[C@@H]1CCCNC1] | W1 | 478.3 |
| 36 | (R)-N-(piperidin-3-yl)-4- (pyridin-3-ylamino)-N- (quinolin-2-yl)benzamide | 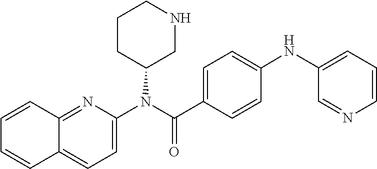 [SMILES: O=C(c1ccc(Nc2cccnc2)cc1)N(c1ccc2ccccc2n1)[C@@H]1CCCNC1] | Q1.5/Q2.1 | 424.3 |
| 37 | (R)-4-(1H-imidazol-1-yl)- N-(piperidin-3-yl)-N- (quinolin-2-yl)benzamide | 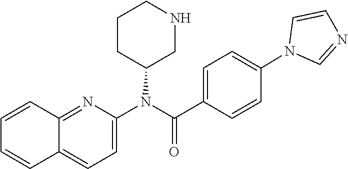 [SMILES: O=C(c1ccc(-n2ccnc2)cc1)N(c1ccc2ccccc2n1)[C@@H]1CCCNC1] | W3 | 398.3 |
| 38 | (R)-4-(3H- [1,2,3]triazolo[4,5- b]pyridin-3-yl)-2-chloro- N-(isoquinolin-3-yl)-N- (piperidin-3-yl)benzamide | 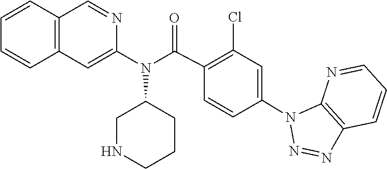 [SMILES: O=C(c1ccc(-n2nnc3cccnc32)cc1Cl)N(c1cc2ccccc2cn1)[C@@H]1CCCNC1] | W1 | 484.3 |
| 39 | (R)-4-(3H- [1,2,3]triazolo[4,5- b]pyridin-3-yl)-2-fluoro-N- (isoquinolin-3-yl)-N- (piperidin-3-yl)benzamide | 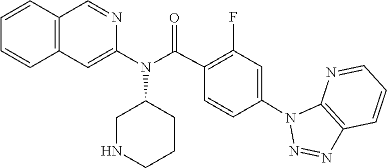 [SMILES: O=C(c1ccc(-n2nnc3cccnc32)cc1F)N(c1cc2ccccc2cn1)[C@@H]1CCCNC1] | W1 | 468.3 |
| 40 | (R)-4-(3H- [1,2,3]triazolo[4,5- b]pyridin-3-yl)-N-(3- methylpyridin-2-yl)-N- (piperidin-3-yl)benzamide | 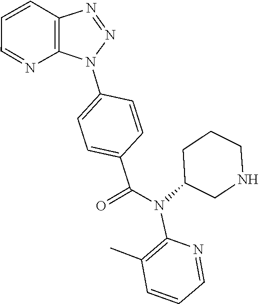 [SMILES: Cc1cccnc1N(C(=O)c1ccc(-n2nnc3cccnc32)cc1)[C@@H]1CCCNC1] | W1 | 414.2 |
| 41 | (R)-4-(3H- [1,2,3]triazolo[4,5- b]pyridin-3-yl)-2-chloro- N-(3-methylpyridin-2-yl)- N-(piperidin-3- yl)benzamide | 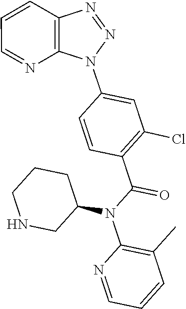 [SMILES: Cc1cccnc1N(C(=O)c1ccc(-n2nnc3cccnc32)cc1Cl)[C@@H]1CCCNC1] | W1 | 448.3 |
| 42 | (R)-4-(3H- [1,2,3]triazolo[4,5- b]pyridin-3-yl)-2-fluoro-N- (3-methylpyridin-2-yl)-N- (piperidin-3-yl)benzamide | 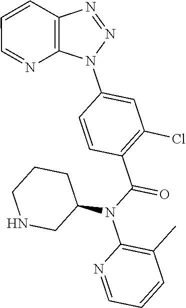 [SMILES: Cc1cccnc1N(C(=O)c1ccc(-n2nnc3cccnc32)cc1Cl)[C@@H]1CCCNC1] | W1 | 432.3 |
| 43 | (R)-4-(3H- [1,2,3]triazolo[4,5- b]pyridin-3-yl)-2-methyl- N-(3-methylpyridin-2-yl)- N-(piperidin-3- yl)benzamide | 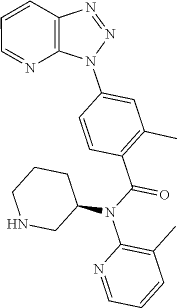 [SMILES: Cc1cc(-n2nnc3cccnc32)ccc1C(=O)N(c1ncccc1C)[C@@H]1CCCNC1] | W1 | 428.3 |
| 44 | (R)-4-(3H- [1,2,3]triazolo[4,5- b]pyridin-3-yl)-2-fluoro-N- (piperidin-3-yl)-N- (pyridin-2-yl)benzamide | 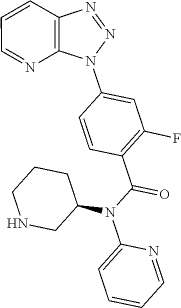 [SMILES: O=C(c1ccc(-n2nnc3cccnc32)cc1F)N(c1ccccn1)[C@@H]1CCCNC1] | W3 | 418.0 |
| 45 | (R)-4-(3H- [1,2,3]triazolo[4,5- b]pyridin-3-yl)-2-ethyl-N- (piperidin-3-yl)-N- (pyridin-2-yl)benzamide | 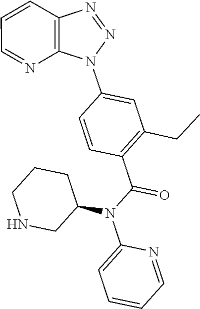 [SMILES: CCc1cc(-n2nnc3cccnc32)ccc1C(=O)N(c1ccccn1)[C@@H]1CCCNC1] | W3 | 428.3 |
| 46 | (R)-4-(3H- [1,2,3]triazolo[4,5- b]pyridin-3-yl)-2-chloro- N-(piperidin-3-yl)-N- (quinolin-2-yl)benzamide | 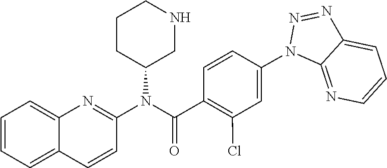 [SMILES: O=C(c1ccc(-n2nnc3cccnc32)cc1Cl)N(c1ccc2ccccc2n1)[C@@H]1CCCNC1] | W1 | 484.0 |
| 47 | (R)-4-(3H- [1,2,3]triazolo[4,5- b]pyridin-3-yl)-2-methyl- N-(8-methylisoquinolin-1- yl)-N-(piperidin-3- yl)benzamide | 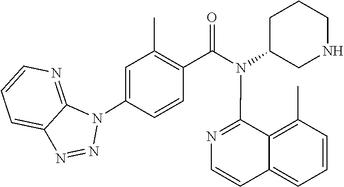 [SMILES: Cc1cc(-n2nnc3cccnc32)ccc1C(=O)N(c1nccc2cccc(C)c12)[C@@H]1CCCNC1] | W1 | 478.0 |
| 48 | (R)-4-(3H- [1,2,3]triazolo[4,5- b]pyridin-3-yl)-2-ethyl-N- (8-methylisoquinolin-1-yl)- N-(piperidin-3- yl)benzamide | 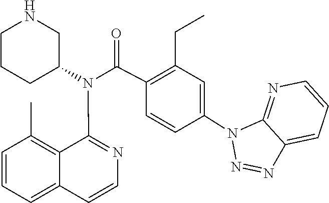 [SMILES: CCc1cc(-n2nnc3cccnc32)ccc1C(=O)N(c1nccc2cccc(C)c12)[C@@H]1CCCNC1] | W1 | 492.0 |
| 49 | (R)-4-(3H- [1,2,3]triazolo[4,5- b]pyridin-3-yl)-2-methoxy- N-(8-methylisoquinolin-1- yl)-N-(piperidin-3- yl)benzamide | 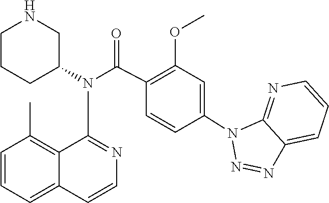 [SMILES: COc1cc(-n2nnc3cccnc32)ccc1C(=O)N(c1nccc2cccc(C)c12)[C@@H]1CCCNC1] | W1 | 494.3 |
| 50 | (R)-4-(3H- [1,2,3]triazolo[4,5- b]pyridin-3-yl)-N-(8- methylisoquinolin-1-yl)-N- (piperidin-3-yl)benzamide | 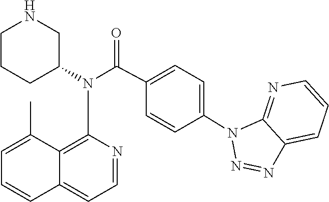 [SMILES: Cc1cccc2ccnc(N(C(=O)c3ccc(-n4nnc5cccnc54)cc3)[C@@H]3CCCNC3)c12] | W1 | 464.0 |
| 51 | (R)-N-(8- methylisoquinolin-1-yl)-N- (piperidin-3-yl)-4- (pyrimidin-2- ylamino)benzamide | 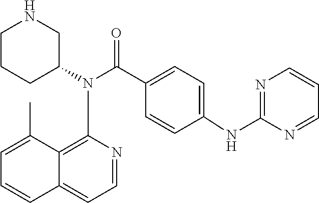 [SMILES: Cc1cccc2ccnc(N(C(=O)c3ccc(Nc4ncccn4)cc3)[C@@H]3CCCNC3)c12] | W1 | 439.0 |
| 52 | (R)-4-(3H- [1,2,3]triazolo[4,5- b]pyridin-3-yl)-2-fluoro-N- (3-methyl-3H- [1,2,3]triazolo[4,5- c]pyridin-4-yl)-N- (piperidin-3-yl)benzamide | 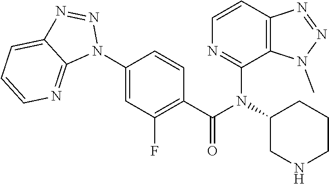 [SMILES: Cn1nnc2ccnc(N(C(=O)c3ccc(-n4nnc5cccnc54)cc3F)[C@@H]3CCCNC3)c21] | W1 | 473.0 |
| 53 | (R)-4-(3H- [1,2,3]triazolo[4,5- b]pyridin-3-yl)-2-chloro- N-(8-methylisoquinolin-1- yl)-N-(piperidin-3- yl)benzamide | 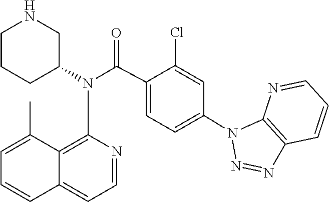 [SMILES: Cc1cccc2ccnc(N(C(=O)c3ccc(-n4nnc5cccnc54)cc3Cl)[C@@H]3CCCNC3)c12] | W1 | 498.0 |
| 54 | (R)-N-(8- methylisoquinolin-1-yl)-N- (piperidin-3-yl)-4-(1H- 1,2,3-triazol-1- yl)benzamide | 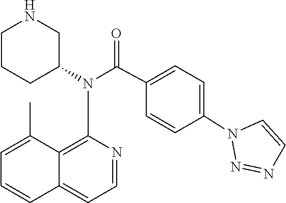 [SMILES: Cc1cccc2ccnc(N(C(=O)c3ccc(-n4ccnn4)cc3)[C@@H]3CCCNC3)c12] | W1 | 413.2 |
| 55 | (R)-4-(6-cyanopyrazin-2- yl)-N-(8- methylisoquinolin-1-yl)-N- (piperidin-3-yl)benzamide | 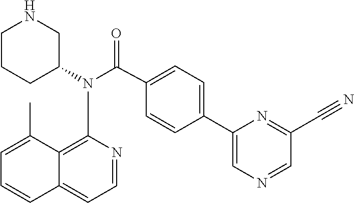 [SMILES: Cc1cccc2ccnc(N(C(=O)c3ccc(-c4cncc(C#N)n4)cc3)[C@@H]3CCCNC3)c12] | W1 | 449.2 |
| 56 | (R)-N-(8- methylisoquinolin-1-yl)-N- (piperidin-3-yl)-4- (pyrimidin-5-yl)benzamide | 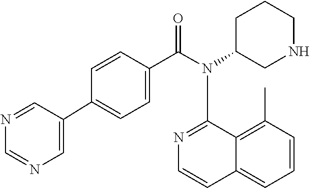 [SMILES: Cc1cccc2ccnc(N(C(=O)c3ccc(-c4cncnc4)cc3)[C@@H]3CCCNC3)c12] | W1 | 424.0 |
| 57 | (R)-4-(2-methyl-1H- benzo[d]imidazol-1-yl)-N- (8-methylisoquinolin-1-yl)- N-(piperidin-3- yl)benzamide | 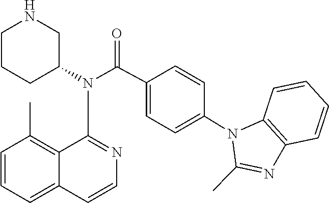 [SMILES: Cc1cccc2ccnc(N(C(=O)c3ccc(-n4c(C)nc5ccccc54)cc3)[C@@H]3CCCNC3)c12] | W1 | 476.0 |
| 58 | (R)-N-(3-methylpyrazin-2- yl)-N-(piperidin-3-yl)-4- (pyrimidin-2-yl)piperazine- 1-carboxamide | 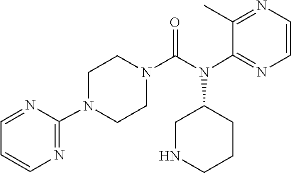 [SMILES: Cc1nccnc1N(C(=O)N1CCN(c2ncccn2)CC1)[C@@H]1CCCNC1] | W5 | 383.0 |
| 59 | (R)-N-(8- methylisoquinolin-1-yl)-N- (piperidin-3-yl)-4-(1H- pyrrol-1-yl)piperidine-1- carboxamide | 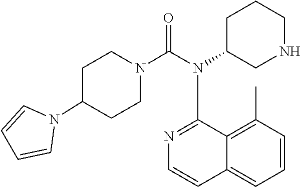 [SMILES: Cc1cccc2ccnc(N(C(=O)N3CCC(n4cccc4)CC3)[C@@H]3CCCNC3)c12] | W5 | 418.0 |
| 60 | (R)-4-(isoxazol-5-yl)-N-(8- methylisoquinolin-1-yl)-N- (piperidin-3-yl)piperidine- 1-carboxamide | 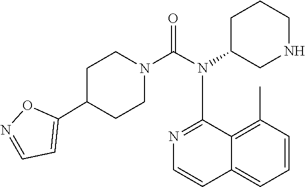 [SMILES: Cc1cccc2ccnc(N(C(=O)N3CCC(c4ccno4)CC3)[C@@H]3CCCNC3)c12] | W5 | 420.0 |
| 61 | (R)-N-(8- methylisoquinolin-1-yl)-4- (3-methylisoxazol-5-yl)-N- (piperidin-3-yl)piperidine- 1-carboxamide | 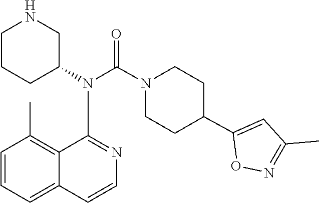 [SMILES: Cc1cc(C2CCN(C(=O)N(c3nccc4cccc(C)c34)[C@@H]3CCCNC3)CC2)on1] | W5 | 434.0 |
| 62 | (R)-N-(8- methylisoquinolin-1-yl)-N- (piperidin-3-yl)-4-(2H- tetrazol-2-yl)piperidine-1- carboxamide | 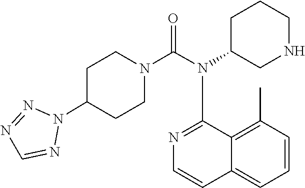 [SMILES: Cc1cccc2ccnc(N(C(=O)N3CCC(n4ncnn4)CC3)[C@@H]3CCCNC3)c12] | W5 | 421.0 |
| 63 | (R)-N-(8- methylisoquinolin-1-yl)-N- (piperidin-3-yl)-4-(1H- 1,2,3-triazol-1- yl)piperidine-1- carboxamide | 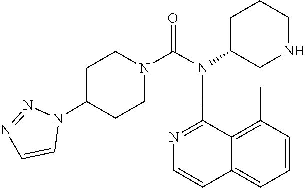 [SMILES: Cc1cccc2ccnc(N(C(=O)N3CCC(n4ccnn4)CC3)[C@@H]3CCCNC3)c12] | W5 | 420.0 |
| 64 | (R)-N-(1-methyl-1H- pyrrolo[2,3-c]pyridin-5- yl)-N-(piperidin-3-yl)-4- (pyridin-4- ylamino)benzamide | 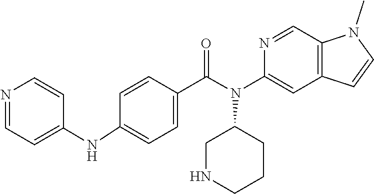 [SMILES: Cn1ccc2cc(N(C(=O)c3ccc(Nc4ccncc4)cc3)[C@@H]3CCCNC3)ncc21] | Q1.8/Q2.1 | 427.3 |
| 65 | (R)-N-(piperidin-3-yl)-4- (pyridin-4-ylamino)-N- (quinolin-2-yl)benzamide | 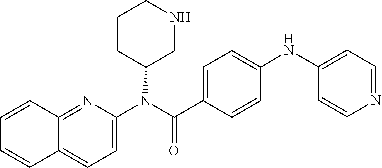 [SMILES: O=C(c1ccc(Nc2ccncc2)cc1)N(c1ccc2ccccc2n1)[C@@H]1CCCNC1] | Q1.8/Q2.1 | 424.3 |
| 66 | (R)-N-(isoquinolin-3-yl)- N-(piperidin-3-yl)-4- (pyridin-3- ylamino)benzamide | 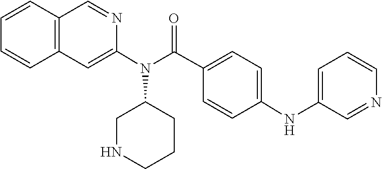 [SMILES: O=C(c1ccc(Nc2cccnc2)cc1)N(c1cc2ccccc2cn1)[C@@H]1CCCNC1] | Q1.5/Q2.1 | 424.4 |
| 67 | (R)-N-(isoquinolin-3-yl)- N-(piperidin-3-yl)-4- (pyridin-4- ylamino)benzamide | 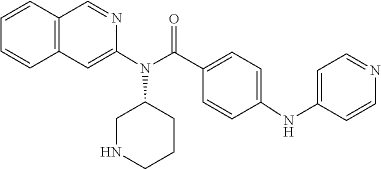 [SMILES: O=C(c1ccc(Nc2ccncc2)cc1)N(c1cc2ccccc2cn1)[C@@H]1CCCNC1] | Q1.8/Q2.1 | 424.3 |
| 68 | (R)-N-(isoquinolin-3-yl)- 4′-methoxy-N-(piperidin-3- yl)-[1,1′-biphenyl]-4- carboxamide | 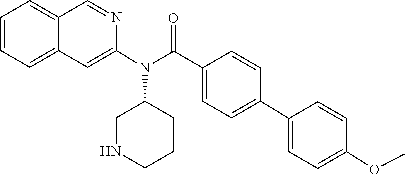 [SMILES: COc1ccc(-c2ccc(C(=O)N(c3cc4ccccc4cn3)[C@@H]3CCCNC3)cc2)cc1] | Q1.1/Q2.1 | 438.4 |
| 69 | (R)-3-fluoro-N- (isoquinolin-3-yl)-4′- methoxy-N-(piperidin-3- yl)-[1,1′-biphenyl]-4- carboxamide | 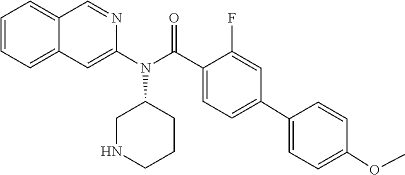 [SMILES: COc1ccc(-c2ccc(C(=O)N(c3cc4ccccc4cn3)[C@@H]3CCCNC3)c(F)c2)cc1] | Q1.1/Q2.1 | 456.4 |
| 70 | (R)-2-fluoro-N- (isoquinolin-3-yl)-4-(1- methyl-1H-pyrazol-4-yl)- N-(piperidin-3- yl)benzamide | 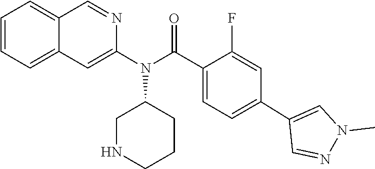 [SMILES: Cn1cc(-c2ccc(C(=O)N(c3cc4ccccc4cn3)[C@@H]3CCCNC3)c(F)c2)cn1] | Q1.1/Q2.1 | 430.3 |
| 71 | (R)-2-fluoro-N- (isoquinolin-3-yl)-N- (piperidin-3-yl)-4-(pyridin- 2-yl)benzamide | 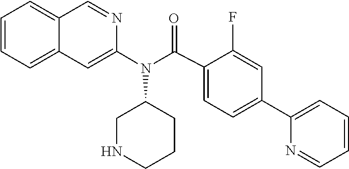 [SMILES: O=C(c1ccc(-c2ccccn2)cc1F)N(c1cc2ccccc2cn1)[C@@H]1CCCNC1] | Q1.4/Q2.1 | 427.2 |
| 72 | (R)-2-fluoro-N- (isoquinolin-3-yl)-N- (piperidin-3-yl)-4-(pyridin- 3-yl)benzamide | 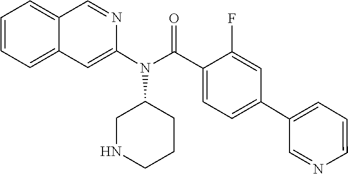 | Q1.1/Q2.1 | 427.4 |
| 73 | (R)-2-fluoro-N- (isoquinolin-3-yl)-N- (piperidin-3-yl)-4-(pyridin- 4-yl)benzamide | 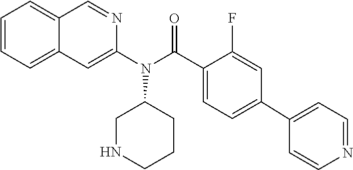 [SMILES: O=C(c1ccc(-c2ccncc2)cc1F)N(c1cc2ccccc2cn1)[C@@H]1CCCNC1] | Q1.1/Q2.1 | 427.3 |
| 74 | (R)-N-(isoquinolin-3-yl)- N-(piperidin-3-yl)-[3,4′- bipyridine]-6-carboxamide | 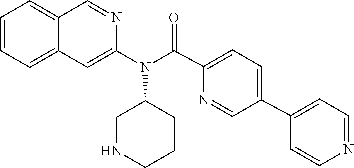 [SMILES: O=C(c1ccc(-c2ccncc2)cn1)N(c1cc2ccccc2cn1)[C@@H]1CCCNC1] | Q1.1/Q2.1 | 410.2 |
| 75 | (R)-N-(isoquinolin-3-yl)- N-(piperidin-3-yl)-3- ((pyridin-3- ylmethyl)amino)benzamide | 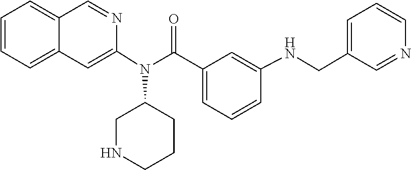 [SMILES: O=C(c1cccc(NCc2cccnc2)c1)N(c1cc2ccccc2cn1)[C@@H]1CCCNC1] | Q1.7/Q2.1 | 438.3 |
| 76 | (R)-N-(1-methyl-1H- pyrrolo[2,3-c]pyridin-5- yl)-N-(piperidin-3-yl)-3- (pyridin-4-yl)benzamide | 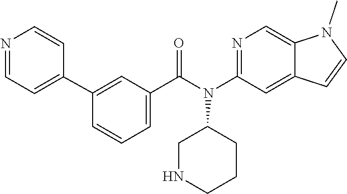 [SMILES: Cn1ccc2cc(N(C(=O)c3cccc(-c4ccncc4)c3)[C@@H]3CCCNC3)ncc21] | Q1.1/Q2.1 | 412.3 |
| 77 | (R)-4-(1-methyl-1H- pyrazol-4-yl)-N-(3- methylpyridin-2-yl)-N- (piperidin-3-yl)benzamide | 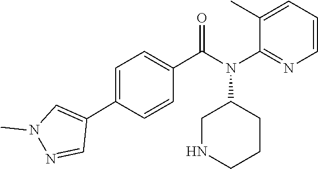 | Q1.1/Q2.1 | 376.2 |
| 78 | (R)-N-(3-methylpyridin-2- yl)-N-(piperidin-3-yl)-4- (pyridin-2-yl)benzamide | 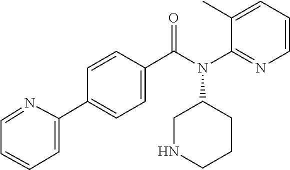 | Q1.4/Q2.1 | 373.3 |
| 79 | (R)-N-(3-methylpyridin-2- yl)-N-(piperidin-3-yl)-4- (pyridin-4-yl)benzamide | 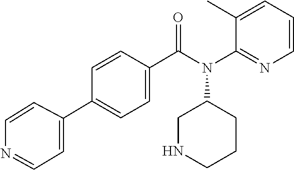 | Q1.1/Q2.1 | 373.2 |
| 80 | (R)-4-((1-methylpiperidin- 4-yl)amino)-N-(3- methylpyridin-2-yl)-N- (piperidin-3-yl)benzamide | 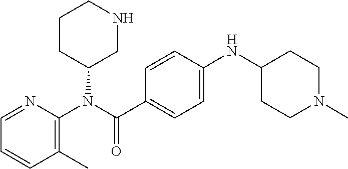 [SMILES: Cc1cccnc1N(C(=O)c1ccc(NC2CCN(C)CC2)cc1)[C@@H]1CCCNC1] | Q1.5/Q2.1 | 408.3 |
| 81 | (R)-3-(1-methyl-1H- pyrazol-4-yl)-N-(3- methylpyridin-2-yl)-N- (piperidin-3-yl)benzamide | 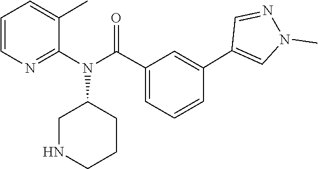 [SMILES: Cc1cccnc1N(C(=O)c1cccc(-c2cnn(C)c2)c1)[C@@H]1CCCNC1] | Q1.1/Q2.1 | 376.3 |
| 82 | (R)-N-(3-methylpyridin-2- yl)-N-(piperidin-3-yl)-3- (pyridin-3-yl)benzamide | 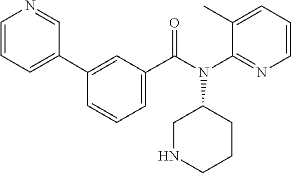 [SMILES: Cc1cccnc1N(C(=O)c1cccc(-c2cccnc2)c1)[C@@H]1CCCNC1] | Q1.1/Q2.1 | 373.3 |
| 83 | (R)-4′-methoxy-N-(1- methyl-1H-pyrrolo[2,3- c]pyridin-5-yl)-N- (piperidin-3-yl)-[1,1′- biphenyl]-4-carboxamide | 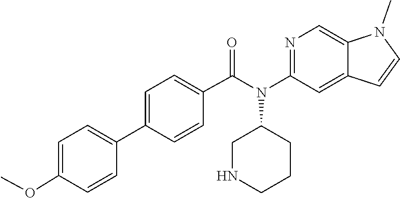 [SMILES: COc1ccc(-c2ccc(C(=O)N(c3cc4ccn(C)c4cn3)[C@@H]3CCCNC3)cc2)cc1] | Q1.1/Q2.1 | 441.3 |
| 84 | (R)-N-(piperidin-3-yl)-N- (quinolin-2-yl)-[2,3′- bipyridine]-6′-carboxamide | 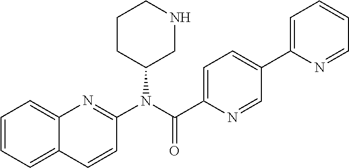 [SMILES: O=C(c1ccc(-c2ccccn2)cn1)N(c1ccc2ccccc2n1)[C@@H]1CCCNC1] | Q1.4/Q2.1 | 410.2 |
| 85 | (R)-4′-methoxy-N- (piperidin-3-yl)-N- (quinolin-2-yl)-[1,1′- biphenyl]-3-carboxamide | 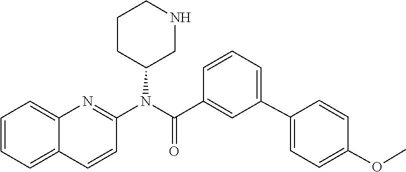 [SMILES: COc1ccc(-c2cccc(C(=O)N(c3ccc4ccccc4n3)[C@@H]3CCCNC3)c2)cc1] | Q1.1/Q2.1 | 438.3 |
| 86 | (R)-3-fluoro-4′-methoxy- N-(1-methyl-1H- pyrrolo[2,3-c]pyridin-5- yl)-N-(piperidin-3-yl)- [1,1′-biphenyl]-4- carboxamide | 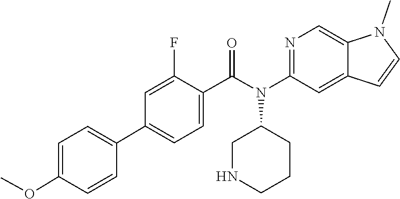 [SMILES: COc1ccc(-c2ccc(C(=O)N(c3cc4ccn(C)c4cn3)[C@@H]3CCCNC3)c(F)c2)cc1] | Q1.1/Q2.1 | 459.3 |
| 87 | (R)-2-fluoro-4-(1-methyl- 1H-pyrazol-4-yl)-N-(1- methyl-1H-pyrrolo[2,3- c]pyridin-5-yl)-N- (piperidin-3-yl)benzamide | 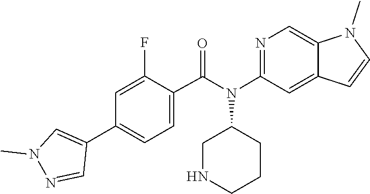 [SMILES: Cn1cc(-c2ccc(C(=O)N(c3cc4ccn(C)c4cn3)[C@@H]3CCCNC3)c(F)c2)cn1] | Q1.1/Q2.1 | 433.3 |
| 88 | (R)-2-fluoro-N-(1-methyl- 1H-pyrrolo[2,3-c]pyridin- 5-yl)-N-(piperidin-3-yl)-4- (pyridin-3-yl)benzamide | 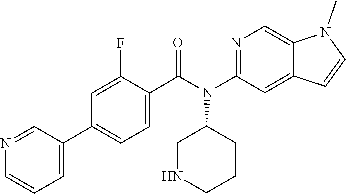 [SMILES: Cn1ccc2cc(N(C(=O)c3ccc(-c4cccnc4)cc3F)[C@@H]3CCCNC3)ncc21] | Q1.1/Q2.1 | 430.3 |
| 89 | (R)-2-fluoro-N- (isoquinolin-3-yl)-4- (methyl(pyridin-3- yl)amino)-N-(piperidin-3- yl)benzamide | 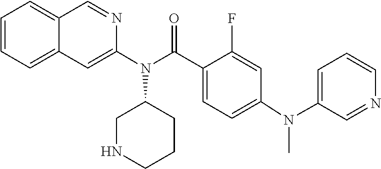 [SMILES: CN(c1cccnc1)c1ccc(C(=O)N(c2cc3ccccc3cn2)[C@@H]2CCCNC2)c(F)c1] | Q1.5/Q2.1 | 456.3 |
| 90 | (R)-N-(3-methylpyridin-2- yl)-4-(oxetan-3-ylamino)- N-(piperidin-3- yl)benzamide | 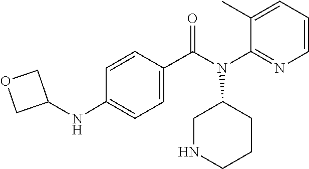 [SMILES: Cc1cccnc1N(C(=O)c1ccc(NC2COC2)cc1)[C@@H]1CCCNC1] | Q1.5/Q2.3 | 367.3 |
| 91 | (R)-4-((1-methylpiperidin- 4-yl)amino)-N-(piperidin- 3-yl)-N-(quinolin-2- yl)benzamide | 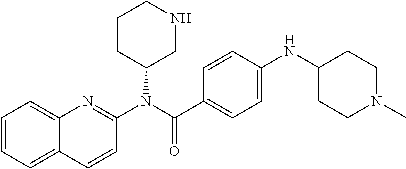 [SMILES: CN1CCC(Nc2ccc(C(=O)N(c3ccc4ccccc4n3)[C@@H]3CCCNC3)cc2)CC1] | Q1.5/Q2.1 | 444.3 |
| 92 | (R)-2-fluoro-N-(piperidin- 3-yl)-4-(pyridin-3- ylamino)-N-(quinolin-2- yl)benzamide | 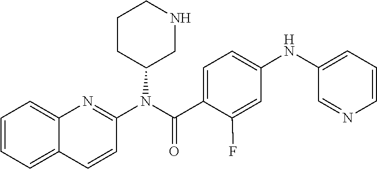 [SMILES: O=C(c1ccc(Nc2cccnc2)cc1F)N(c1ccc2ccccc2n1)[C@@H]1CCCNC1] | Q1.5/Q2.1 | 442.3 |
| 93 | (R)-2-fluoro-N-(1-methyl- 1H-pyrrolo[2,3-c]pyridin- 5-yl)-N-(piperidin-3-yl)-4- (pyridin-4-yl)benzamide | 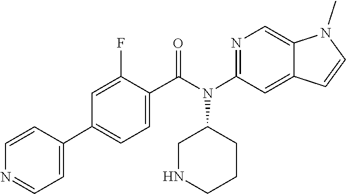 [SMILES: Cn1ccc2cc(N(C(=O)c3ccc(-c4ccncc4)cc3F)[C@@H]3CCCNC3)ncc21] | Q1.1/Q2.1 | 430.3 |
| 94 | (R)-N-(isoquinolin-3-yl)- N-(piperidin-3-yl)-4- (pyridin-2- ylamino)benzamide | 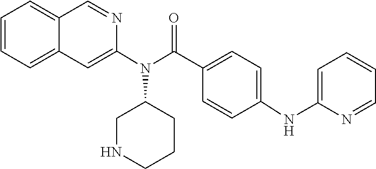 [SMILES: O=C(c1ccc(Nc2ccccn2)cc1)N(c1cc2ccccc2cn1)[C@@H]1CCCNC1] | Q1.7/Q2.1 | 424.3 |
| 95 | (R)-N-(1-methyl-1H- pyrrolo[2,3-c]pyridin-5- yl)-N-(piperidin-3-yl)-4- (pyridin-2- ylamino)benzamide | 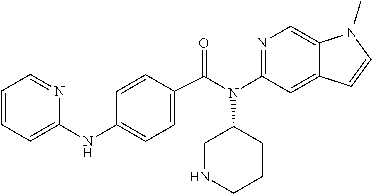 [SMILES: Cn1ccc2cc(N(C(=O)c3ccc(Nc4ccccn4)cc3)[C@@H]3CCCNC3)ncc21] | Q1.7/Q2.1 | 427.3 |
| 96 | (R)-2-hydroxy-N-(1- methyl-1H-pyrrolo[2,3- c]pyridin-5-yl)-N- (piperidin-3-yl)-4- (pyrimidin-5- ylamino)benzamide | 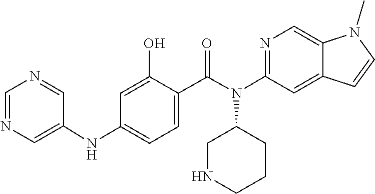 [SMILES: Cn1ccc2cc(N(C(=O)c3ccc(Nc4cncnc4)cc3O)[C@@H]3CCCNC3)ncc21] | Q1.5/Q2.1 | 444.3 |
| 97 | (R)-4-(3H- [1,2,3]triazolo[4,5- b]pyridin-3-yl)-2-fluoro-N- (6-(1-methyl-1H-pyrazol- 4-yl)isoquinolin-1-yl)-N- (piperidin-3-yl)benzamide | 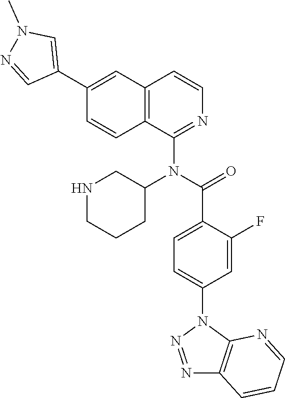 [SMILES: Cn1cc(-c2ccc3c(N(C(=O)c4ccc(-n5nnc6cccnc65)cc4F)C4CCCNC4)nccc3c2)cn1] | | 548.3 |

### Synthesis of (R)-4-(3H-[1,2,3]triazolo[4,5-b]pyridin-3-yl)-2-fluoro-N-(6-(1-methyl-1H-pyrazol-4-yl)isoquinolin-1-yl)-N-(piperidin-3-yl)benzamide (Example 97)

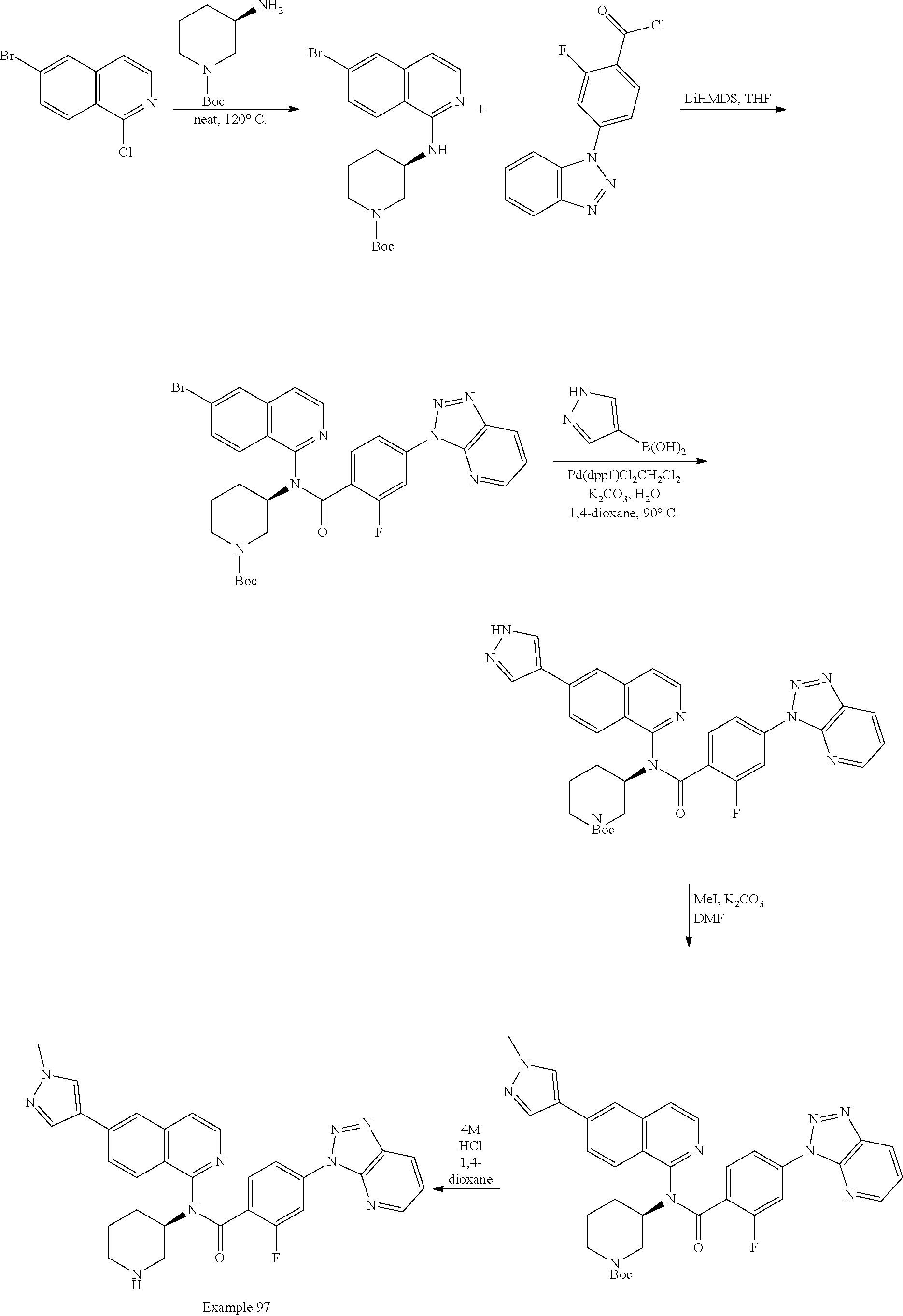

### Step 1:

6-Bromo-1-chloroisoquinoline (1.00 g, 4.151 mmol) and tert-butyl (R)-3-aminopiperidine-1-carboxylate (4.10 g, 20.75 mmol, specific optical rotation=−35° at 20° C. (c=5 in MeOH)) were placed in a sealed tube, and the reaction mixture was stirred at 120° C. for 16 h. The reaction mixture was allowed to cool and concentrated. The residue was purified by Combi-flash using 100% EtOAc to afford tert-butyl (R)-3-((6-bromoisoquinolin-1-yl)amino)piperidine-1-carboxylate (250 mg, 16%) as a thick oil.

### Step 2:

To a solution of tert-butyl (R)-3-((6-bromoisoquinolin-1-yl)amino)piperidine-1-carboxylate (250 mg, 0.617 mmol) in THF (50 mL) at 0° C. was added 1.5M LiHMDS in THF (1.2 mL, 1.851 mmol). The reaction mixture was stirred at 0° C. for 10 min and followed by addition of a solution of 4-(3H-[1,2,3]triazolo[4,5-b]pyridin-3-yl)-2-fluorobenzoyl chloride (204 mg, 0.740 mmol) in THF (5 mL). The reaction mixture was stirred for 30 min before it was quenched with ice cold water and extracted with EtOAc (2×50 mL). The combined organic layer was washed with brine, dried over Na₂SO₄, and concentrated. The crude product was purified by Combi-flash using 80% EtOAc first and further purified by Prep-HPLC [Conditions: Column: Luna OMEGA C-18 (250×21.2×5 μm), Mobile phase A: 0.1% formic acid in water (100%), Mobile phase B: MeCN (100%), Gradient: T/% B: 0/65, 10/95, Flow: 15 mL/min, Dilution: MeCN/H₂O/THF]. Pure fractions were dried by lyophilization to afford tert-butyl (R)-3-(4-(3H-[1,2,3]triazolo[4,5-b]pyridin-3-yl)-N-(6-bromoisoquinolin-1-yl)-2-fluorobenzamido)piperidine-1-carboxylate (120 mg) as a white solid.

### Step 3:

To a solution of tert-butyl (R)-3-(4-(3H-[1,2,3]triazolo[4,5-b]pyridin-3-yl)-N-(6-bromoisoquinolin-1-yl)-2-fluorobenzamido)piperidine-1-carboxylate (200 mg, 0.31 mmol) in 1,4-dioxane (4 mL) & H₂O (1 mL) were added (1H-pyrazol-4-yl)boronic acid (69.3 mg, 0.620 mmol) and K₂CO₃ (128.5 mg, 0.93 mmol). The reaction mixture was degassed with nitrogen for 5 minutes. Then, Pd(dppf)Cl₂ DCM (25.3 mg, 0.031 mmol) was added to reaction mixture, and the reaction was stirred at 90° C. for 16 h in a sealed tube. The reaction mixture was diluted with EtOAc (100 mL), filtered through celite, and the filtrate was washed with water. The organic layer was washed with brine, dried over Na₂SO₄, and concentrated under reduced pressure. The residue was further purified by Prep-HPLC (Conditions: Luna OMEGA C-18 (250×21.2×5 μm), Mobile phase A: 0.1% formic acid in water (100%), Mobile phase B: 100% MeCN, Gradient: T/% B: 0/50, 10/85, Flow: 15 mL/min, Dilution: MeCN+H₂O+THF). The pure fractions were concentrated under reduced pressure to afford tert-butyl (R)-3-(N-(6-(1H-pyrazol-4-yl)isoquinolin-1-yl)-4-(3H-[1,2,3]triazolo[4,5-b]pyridin-3-yl)-2-fluorobenzamido)piperidine-1-carboxylate (60 mg, 0.09 mmol, 30%) as a white solid. LC-MS: [M+H⁺]=634.25.

### Step 4:

To a solution of tert-butyl (R)-3-(N-(6-(1H-pyrazol-4-yl)isoquinolin-1-yl)-4-(3H-[1,2,3]triazolo[4,5-b]pyridin-3-yl)-2-fluorobenzamido)piperidine-1-carboxylate (25 mg, 0.045 mmol) in THF (2 mL) at 0° C. was added sodium hydride (4 mg, 0.157 mmol, 60%). The reaction was stirred for 15 min and followed by addition of Mel (11.1 mg, 0.078 mmol). The reaction was stirred at room temperature for 1 h before it was diluted with water (15 mL) and extracted with EtOAc (2×30 mL). The combined organic layer was washed with brine, dried over Na₂SO₄, and concentrated under reduced pressure. The residue was purified by Prep-HPLC (Conditions: Luna OMEGA C-18 (250×21.2×5 μm) Mobile phase A: 0.1% formic acid in water (100%), Mobile phase B: 100% MeCN, Gradient: Time (min)/% B: 0/10, 2/10, 10/65 Flow: 15 mL/min, Dilution: MeCN+H₂O+THF). The pure fractions were combined and dried by lyophilization to afford tert-butyl (R)-3-(4-(3H-[1,2,3]triazolo[4,5-b]pyridin-3-yl)-2-fluoro-N-(6-(1-methyl-1H-pyrazol-4-yl)isoquinolin-1-yl)benzamido)piperidine-1-carboxylate (15 mg) as a white solid. LC-MS: [M+H⁺]=648.30.

### Step 5:

Deprotection method A: To a solution of tert-butyl (R)-3-(4-(3H-[1,2,3]triazolo[4,5-b]pyridin-3-yl)-2-fluoro-N-(6-(1-methyl-1H-pyrazol-4-yl)isoquinolin-1-yl)benzamido)piperidine-1-carboxylate (15 mg, 0.023 mmol) in DCM (2 mL) at 0° C. was added 4M HCl in 1,4-dioxane (2 mL). The reaction was warmed to room temperature and stirred for 2 h. The reaction mixture was concentrated under reduced pressure and dried by lyophilization to afford the title compound hydrochloride salt (9 mg) as a yellow solid. LC-MS: [M+H⁺]=548.30. ¹H NMR (DMSO-d₆, 400 MHZ): δ ppm 8.78 (d, J=4.4 Hz, 1H), 8.65 (d, J=8.4 Hz, 1H), 8.43-8.35 (m, 2H), 8.18-8.15 (m, 1H), 8.10-8.03 (m, 2H), 7.98-7.95 (m, 2H), 7.82-7.80 (m, 1H), 7.73-7.71 (m, 1H), 7.73-7.71 (m, 1H), 7.59-7.56 (m, 1H), 7.42-7.34 (m, 1H), 5.19-5.15 (m, 1H), 3.93-3.89 (m, 1H), 3.88 (s, 3H), 3.87-3.80 (m, 1H), 3.21-3.18 (m, 2H), 1.97-1.91 (m, 1H), 1.79-1.76 (m, 2H), 1.24-1.23 (m, 1H).

### Synthesis of ethyl (R,E)-3-(1-(4-(3H-[1,2,3]triazolo[4,5-b]pyridin-3-yl)-2-fluoro-N-(piperidin-3-yl)benzamido)isoquinolin-6-yl)acrylate (Example 98)

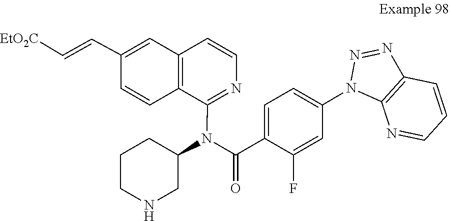 [SMILES: CCOC(=O)/C=C/c1ccc2c(N(C(=O)c3ccc(-n4nnc5cccnc54)cc3F)[C@@H]3CCCNC3)nccc2c1]

Example 98 was prepared according to the procedure described for Example 97 by substituting (1H-pyrazol-4-yl)boronic acid with (E)-(3-ethoxy-3-oxoprop-1-en-1-yl)boronic acid, provided the title compound trifluoroacetic acid salt as an off-white solid. LC-MS: [M+H⁺]=566.35. ¹H NMR (CD₃OD, 400 MHZ) δ 8.73 (d, J=4.8 Hz, 1H), 8.50-8.44 (m, 2H), 8.19 (d, J=9.2 Hz, 1H), 8.04-7.99 (m, 3H), 7.92-7.89 (m, 1H), 7.80-7.72 (m, 2H), 7.52-7.49 (m, 1H), 7.41-7.30 (m, 1H), 6.71 (d, J=16.0 Hz, 1H), 5.31-5.23 (m, 1H), 4.27-4.22 (m, 1H), 3.98-3.95 (m, 1H), 3.81-3.70 (m, 1H), 2.90-2.83 (m, 1H), 3.33-3.00 (m, 1H), 2.90-2.83 (m, 1H), 2.11-1.85 (m, 3H), 1.39-1.28 (m, 4H).

### Synthesis of (R,E)-4-(3H-[1,2,3]triazolo[4,5-b]pyridin-3-yl)-N-(2-(3-((tert-butyldimethylsilyl)oxy)prop-1-en-1-yl)thieno[3,2-c]pyridin-4-yl)-2-fluoro-N-(piperidin-3-yl)benzamide (Example 99)

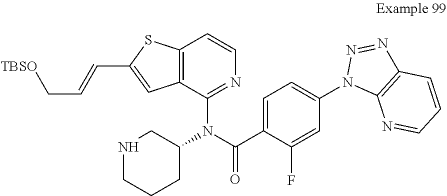 [SMILES: CC(C)(C)[Si](C)(C)OC/C=C/c1cc2c(N(C(=O)c3ccc(-n4nnc5cccnc54)cc3F)[C@@H]3CCCNC3)nccc2s1]

Example 99 was prepared from the precursor of Example of 104 by removing the Boc group with ZnBr₂ to provide the title compound as a white solid. LC-MS: [M+H⁺]=644. ¹H NMR (400 MHZ, DMSO-d₆) δ 8.89-8.77 (m, 1H), 8.74-8.59 (m, 1H), 8.36-8.21 (m, 1H), 8.03-7.94 (m, 1H), 7.93-7.78 (m, 2H), 7.70-7.56 (m, 1H), 7.55-7.49 (m, 1H), 7.48-7.28 (m, 1H), 7.04-6.88 (m, 1H), 6.39-6.20 (m, 1H), 4.74-4.47 (m, 1H), 4.36 (br d, J=1.5 Hz, 2H), 2.91-2.72 (m, 1H), 2.23-2.11 (m, 2H), 1.75-1.41 (m, 3H), 1.29-1.20 (m, 2H), 0.93 (s, 9H), 0.88-0.78 (m, 1H), 0.16-0.01 (m, 6H).

### Synthesis of ethyl (R,E)-3-(1-(4-(1-methyl-1H-1,2,3-triazol-4-yl)-N-(piperidin-3-yl)benzamido)isoquinolin-8-yl)acrylate (Example 100)

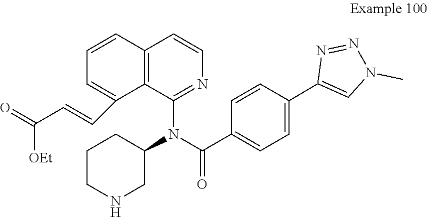 [SMILES: CCOC(=O)/C=C/c1cccc2ccnc(N(C(=O)c3ccc(-c4cn(C)nn4)cc3)[C@@H]3CCCNC3)c12]

Example 100 was prepared according to the procedure described for Example 111 by substituting tert-butyl (R)-3-((8-methylisoquinolin-1-yl)amino)piperidine-1-carboxylate with tert-butyl (R,E)-3-((8-(3-ethoxy-3-oxoprop-1-en-1-yl)isoquinolin-1-yl)amino)piperidine-1-carboxylate, provided the title compound trifluoroacetic acid salt as an off-white solid. LC-MS: [M+H⁺]=532.2.

### Synthesis of (R)—N-(3-(1H-pyrazol-3-yl)pyridin-2-yl)-4-(1-methyl-1H-1,2,3-triazol-4-yl)-N-(piperidin-3-yl)benzamide (Example 101)

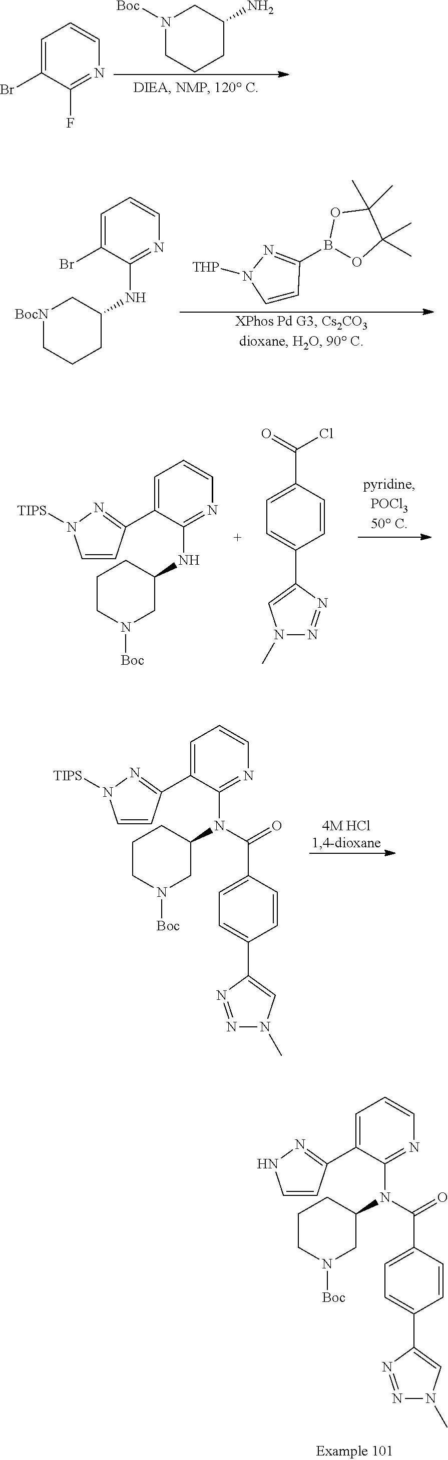

### Step 1:

A mixture of 3-bromo-2-fluoropyridine (5 g, 28.41 mmol), tert-butyl (R)-3-aminopiperidine-1-carboxylate (4.74 g, 23.68 mmol), and DIEA (20.62 mL, 118.38 mmol,) in N-methyl-2-pyrrolidone (75 mL) at 25° C. was purged with N₂ for 3 times, and then the mixture was heated to 120° C. and stirred for 48 h under N₂ atmosphere. The reaction mixture was quenched by addition of water (750 mL) at 25° C., and the product was extracted with ethyl acetate (3×300 mL). The combined organic layer was washed with brine, dried over Na₂SO₄, filtered, and concentrated. The crude residue was purified by column chromatography (SiO₂, Petroleum ether/Ethyl acetate=25/1 to 20/1) to give tert-butyl (3R)-3-[[2-fluoro-4-(1-methyltriazol-4-yl) benzoyl]-[2-(1H-indol-6-yl)thieno[3, 2-c]pyridin-4-yl]amino]piperidine-1-carboxylate (3.1 g, 36%) as a white solid. LC-MS: [M+H⁺]=356.2.

### Step 2:

A mixture of tert-butyl (3R)-3-[[2-fluoro-4-(1-methyltriazol-4-yl) benzoyl]-[2-(1H-indol-6-yl)thieno[3, 2-c] pyridin-4-yl] amino]piperidine-1-carboxylate (500 mg, 1.40 mmol), 1-(tetrahydro-2H-pyran-2-yl)-3-(4,4,5,5-tetramethyl-1,3,2-dioxaborolan-2-yl)-1H-pyrazole (796.76 mg, 2.80 mmol), Cs₂CO₃ (1.37 g, 4.22 mmol) and XPhos Pd G3 (118.8 mg, 170.34 μmol) in dioxane (10 mL) and H₂O (2 mL) at 20° C. was purged with N₂. The mixture was heated to 90° C. and stirred for 12 h under N₂ atmosphere. The reaction mixture was concentrated under reduced pressure. The crude product was purified by reversed-phase Prep-HPLC (neutral condition) to give tert-butyl (3R)-3-((3-(1-(tetrahydro-2H-pyran-2-yl)-1H-pyrazol-3-yl) pyridin-2-yl) amino) piperidine-1-carboxylate (600 mg, 50%) as a white solid. LC-MS: [M+H⁺]=428.4.

### Step 3:

A mixture of 4-(1-methyltriazol-4-yl) benzoic acid (201.99 mg, 994.06 μmol), POCl₃ (44.48 μL, 477.15 μmol), and tert-butyl (3R)-3-[[3-(1-tetrahydropyran-2-ylpyrazol-3-yl)-2-pyridyl] amino] piperidine-1-carboxylate (170 mg, 398 μmol) in pyridine (3 mL) at 25° C. was purged with N₂ for 3 times, and then the mixture was heated to 50° C. and stirred for 3 h under N₂ atmosphere. The reaction mixture was quenched by addition of water (30 mL) at 25° C., and then extracted with ethyl acetate (3×10 mL). The combined organic layer was washed with brine (20 m) L, dried over Na₂SO₄, filtered, and concentrated. The residue was purified by prep-HPLC (column: Waters Xbridge BEH C18 100*30 mm*10 μm; mobile phase: [H₂O (10 mM NH₄HCO₃)-MeCN]; gradient: 35%-65% B over 8.0 min) to give tert-butyl (3R)-3-[4-(1-methyltriazol-4-yl)benzoyl]-[3-(1-tetrahydropyran-2-ylpyrazol-3-yl)-2-pyridyl] amino] piperidine-1-carboxylate (50 mg, 20%) as a white solid.

### Step 4:

A solution of tert-butyl (3R)-3-[[4-(1-methyltriazol-4-yl) benzoyl]-[3-(1-tetrahydropyran-2-ylpyrazol-3-yl)-2-pyridyl] amino] piperidine-1-carboxylate (45 mg, 73.44 μmol) in 4M HCl in dioxane (1 mL) was stirred at 20° C. for 2 h. The reaction mixture was concentrated under reduced pressure. The residue was purified by prep-HPLC (column: Phenomenex luna C18 100*40 mm*5 μm; mobile phase: [H₂O (0.04% HCl)-MeCN]; gradient: 1%-25% B over 8.0 min) to give 4-(1-methyltriazol-4-yl)-N-[(3R)-3-piperidyl]-N-[3-(1H-pyrazol-3-yl)-2-pyridyl] benzamide hydrochloride salt (13.1 mg, 41%) as a white solid. LC-MS: [M+H⁺]=429.3. ¹H NMR (400 MHZ, CD₃OD): δ 8.74 (br s, 1H), 8.36 (d, J=5.5 Hz, 2H), 7.89-7.77 (m, 2H), 7.63 (t, J=7.7 Hz, 1H), 7.56-7.42 (m, 2H), 4.40-4.06 (m, 5H), 2.90 (br d, J=12.0 Hz, 2H), 2.76 (s, 3H), 2.46 (br d, J=12.5 Hz, 1H), 2.35 (br t, J=11.5 Hz, 1H), 2.27-1.99 (m, 4H), 1.81 (br d, J=13.8 Hz, 1H), 1.71-1.55 (m, 1H).

### Synthesis of (R)-4-(3H-[1,2,3]triazolo[4,5-b]pyridin-3-yl)-2-fluoro-N-(piperidin-3-yl)-N-(8-vinylisoquinolin-1-yl)benzamide (Example 102)

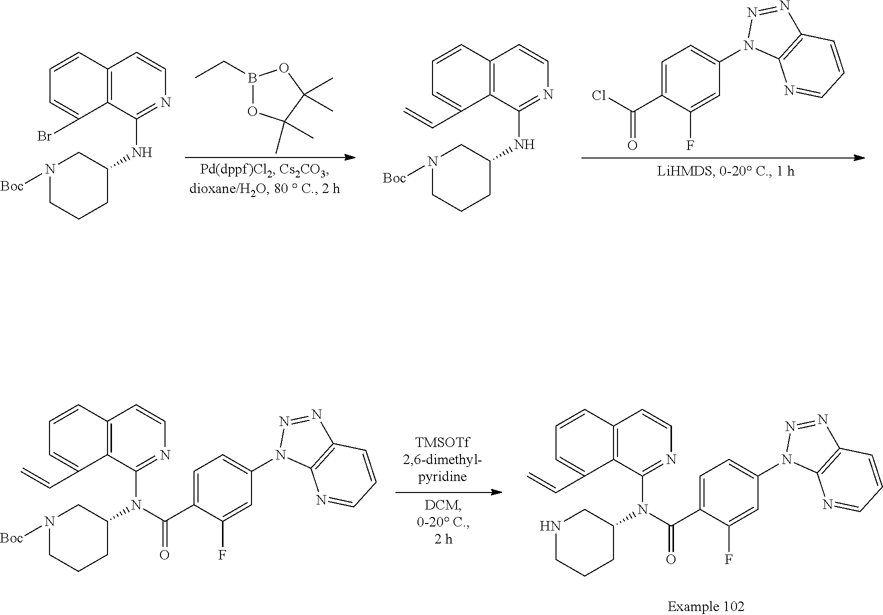

To a solution of tert-butyl (3R)-3-[(8-bromo-1-isoquinolyl)amino]piperidine-1-carboxylate (0.5 g, 1.23 mmol) in dioxane (5 mL) and H₂O (1 mL) was added 4,4,5,5-tetramethyl-2-vinyl-1,3,2-dioxaborolane (313.09 μL, 1.85 mmol), Cs₂CO₃ (1.20 g, 3.69 mmol, 3 eq), and Pd(dppf)Cl₂ (1.80 g, 2.46 mmol) at 20° C. under N₂. The mixture was heated to 80° C. and stirred for 2 h under N₂. The reaction mixture was then filtered and concentrated. The residue was purified by column chromatography (SiO₂, Petroleum ether/Ethyl acetate=5/1 to 3/1) to give tert-butyl (3R)-3-[(8-vinyl-1-isoquinolyl) amino] piperidine-1-carboxylate (0.3 g, 65%) as a yellow oil.

### Step 2:

To a solution of tert-butyl (3R)-3-[(8-vinyl-1-isoquinolyl) amino] piperidine-1-carboxylate (0.1 g, 282.92 μmol) in THF (5 mL) was added LiHMDS (1 M, 848.76 μL) at 0° C. under N₂ atmosphere The mixture was stirred at 0° C. for 0.5 h and followed by a solution of 2-fluoro-4-(triazolo[4,5-b]pyridin-3-yl)benzoyl chloride (101.75 mg, 367.80 μmol) in THF (1 mL) The reaction mixture was stirred at 20° C. for 0.5 h under N₂ atmosphere before it was poured into a saturated aq. NH₄Cl solution (8 mL) very slowly over 10 min. The aqueous phase was extracted with ethyl acetate (3×5 mL), and the combined organic phase was washed with brine (5 mL), dried over anhydrous Na₂SO₄, and concentrated in vacuo to give a residue, which was purified by prep-HPLC (column: Waters Xbridge BEH C18 100*30 mm*10 μm; mobile phase: [A: H₂O (10 mM NH₄HCO₃); B: MeCN]; B %: 40.00%-70.00%, 8.00 min) to give tert-butyl (3R)-3-[[2-fluoro-4-(triazolo[4,5-b]pyridin-3-yl)benzoyl]-(8-vinyl-1-isoquinolyl)amino]piperidine-1-carboxylate (50 mg, 28%) as a white solid.

### Step 3:

To a solution of tert-butyl (3R)-3-[[2-fluoro-4-(triazolo[4,5-b]pyridin-3-yl)benzoyl]-(8-vinyl-1-isoquinolyl)amino]piperidine-1-carboxylate (50 mg, 84.22 μmol) in DCM (1 mL) at 0° C. under N₂ atmosphere was added TMSOTf (91.32 μL, 505.35 μmol,) and 2,6-dimethylpyridine (54.15 mg, 505.35 μmol). The mixture was stirred at 20° C. for 2 h. The reaction mixture was concentrated under reduced pressure to give a residue, which was purified by prep-HPLC (column: Waters Xbridge BEH C18 100*30 mm*10 μm; mobile phase: [A: H₂O (10 mM NH₄HCO₃); B: MeCN]; B %: 45.00%-75.00%, 8.00 min) to give (R)-4-(3H-[1,2,3]triazolo[4,5-b]pyridin-3-yl)-2-fluoro-N-(piperidin-3-yl)-N-(8-vinylisoquinolin-1-yl)benzamide as a white solid. LC-MS: [M+H⁺]=494.2. ¹H NMR (400 MHZ, DMSO-d₆): δ 8.80 (br d, J=3.38 Hz, 1H), 8.66 (br d, J=8.26 Hz, 1H), 8.41-8.48 (m, 1H), 7.81-7.92 (m, 2H), 7.67-7.79 (m, 4H), 7.42-7.60 (m, 2H), 7.13-7.25 (m, 1H), 5.74-5.83 (m, 1H), 5.60 (br d, J=10.88 Hz, 1H), 4.35-4.53 (m, 1H), 3.55 (br d, J=11.38 Hz, 1H), 3.03-3.10 (m, 1H), 2.83 (br d, J=11.76 Hz, 1H), 2.06-2.36 (m, 2H), 1.43-1.76 (m, 3H), 0.88-1.14 (m, 1H).

### Synthesis of (R)-4-(3H-[1,2,3]triazolo[4,5-b]pyridin-3-yl)-2-fluoro-N-(7-(3-hydroxyprop-1-yn-1-yl)isoquinolin-1-yl)-N-(piperidin-3-yl)benzamide (Example 103)

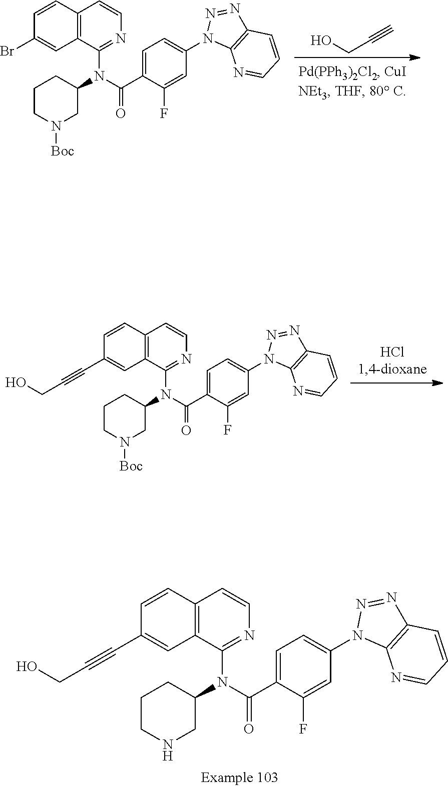

### Step 1:

To a solution of tert-butyl (R)-3-(4-(3H-[1,2,3]triazolo[4,5-b]pyridin-3-yl)-N-(7-bromoisoquinolin-1-yl)-2-fluorobenzamido)piperidine-1-carboxylate (250 mg, 0.386 mmol) in THF (5 mL) at room temperature were added prop-2-yn-1-ol (43 mg, 0.767 mmol) followed by CuI (36 mg, 0.189 mmol) and TEA (0.2 mL). The reaction mixture was purged with nitrogen for 5 min and followed by addition of PdCl₂ (PPh₃) 2 (30 mg, 0.038 mmol). The reaction mixture was heated to 90° C. and stirred for 16 h before it was allowed to cool to room temperature. The reaction mixture was diluted with water (20 mL) and extracted with EtOAc (2×20 mL). The organic layer was washed with brine, dried over Na₂SO₄, and concentrated under reduced pressure. The crude product was purified by Prep-HPLC (Conditions: LUNA, Mobile phase A: 0.1% formic acid in water, Mobile phase B: 100% MeCN, Gradient: T/% B: 0/60, 10/80, Flow: 15 mL/min, Dilution: MeCN+H₂O+THF). Pure fractions were concentrated under reduced pressure and dried by lyophilization to afford tert-butyl (R)-3-(4-(3H-[1,2,3]triazolo[4,5-b]pyridin-3-yl)-2-fluoro-N-(7-(3-hydroxyprop-1-yn-1-yl)isoquinolin-1-yl)benzamido)piperidine-1-carboxylate (30 mg, 0.04 mmol, 30%) as an off-white solid. LC-MS: [M+H⁺]=622.25.

### Step 2:

To a solution of tert-butyl (R)-3-(4-(3H-[1,2,3]triazolo[4,5-b]pyridin-3-yl)-2-fluoro-N-(7-(3-hydroxyprop-1-yn-1-yl)isoquinolin-1-yl)benzamido)piperidine-1-carboxylate (30 mg, 0.048 mmol) in DCM (3 mL) at 0° C. was added 4M HCl in 1,4-dioxane (0.2 mL). The reaction was allowed to warm to room temperature and stirred for 1 h. The reaction mixture was concentrated under reduced pressure and dried by lyophilization to afford the title compound hydrochloride salt (30 mg, 30%) as an off-white solid. LC-MS: [M+H⁺]=522.25.

¹H NMR (CD₃OD, 400 MHZ): δ 8.75 (d, J=4.4 Hz, 1H), 8.51-8.47 (m, 2H), 8.22 (s, 1H), 8.06 (d, J=10.8 Hz, 1H), 7.94-7.92 (m, 1H), 7.82-7.80 (m, 1H), 7.76-7.75 (m, 1H), 7.71-7.65 (m, 1H), 7.54-7.51 (m, 1H), 7.30-7.26 (m, 1H), 5.34-5.22 (m, 1H), 4.50 (s, 2H), 3.97-3.94 (m, 1H), 3.73-3.67 (m, 1H), 3.92-3.83 (m, 1H), 2.01-1.91 (m, 3H), 1.68-1.52 (m, 1H), 1.33-1.28 (m, 1H).

### Synthesis of 2-fluoro-N-[3-[(E)-4-hydroxybut-1-enyl]-2-pyridyl]-N-[(3R)-3-piperidyl]-4-(triazolo[4,5-b]pyridin-3-yl)benzamide (Example 104)

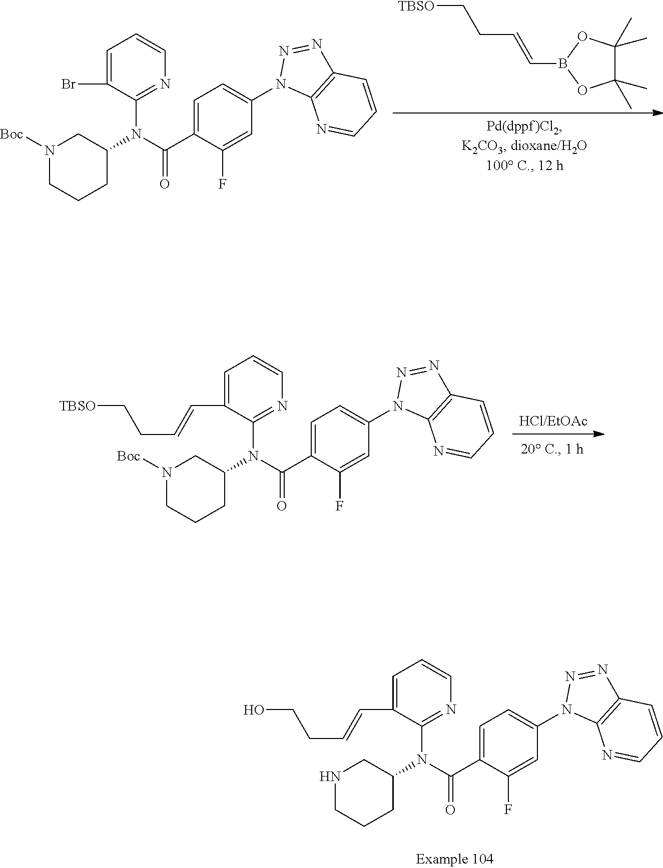 [SMILES: CC(C)(C)OC(=O)N1CCC[C@@H](N(C(=O)c2ccc(-n3nnc4cccnc43)cc2F)c2ncccc2Br)C1 ; CC1(C)OB(/C=C/CCO[Si](C)(C)C(C)(C)C)OC1(C)C ; CC(C)(C)OC(=O)N1CCC[C@@H](N(C(=O)c2ccc(-n3nnc4cccnc43)cc2F)c2ncccc2/C=C/CCO[Si](C)(C)C(C)(C)C)C1 ; O=C(c1ccc(-n2nnc3cccnc32)cc1F)N(c1ncccc1/C=C/CCO)[C@@H]1CCCNC1]

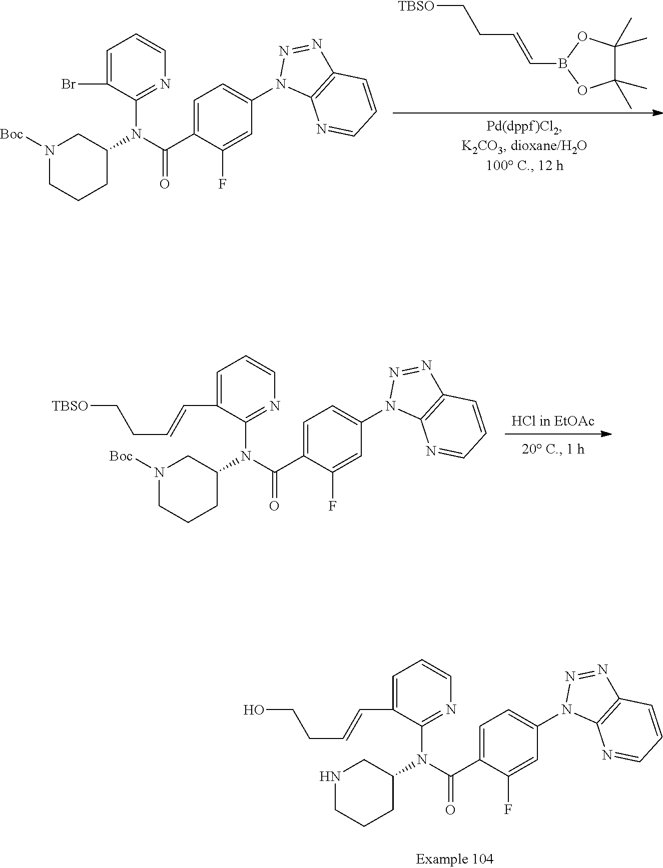 [SMILES: CC(C)(C)OC(=O)N1CCC[C@@H](N(C(=O)c2ccc(-n3nnc4cccnc43)cc2F)c2ncccc2Br)C1 ; CC1(C)OB(/C=C/CCO[Si](C)(C)C(C)(C)C)OC1(C)C ; CC(C)(C)OC(=O)N1CCC[C@@H](N(C(=O)c2ccc(-n3nnc4cccnc43)cc2F)c2ncccc2/C=C/CCO[Si](C)(C)C(C)(C)C)C1 ; O=C(c1ccc(-n2nnc3cccnc32)cc1F)N(c1ncccc1/C=C/CCO)[C@@H]1CCCNC1]

### Step 1:

A mixture of tert-butyl-dimethyl-[(E)-4-(4,4,5,5-tetramethyl-1,3,2-dioxaborolan-2-yl)but-3-enoxy]silane (157.09 mg, 502.98 μmol), tert-butyl (3R)-3-[(3-bromo-2-pyridyl)-[2-fluoro-4-(triazolo[4,5-b]pyridin-3-yl)benzoyl]amino]piperidine-1-carboxylate (166.67 mg, 251.49 μmol), K₂CO₃ (104.27 mg, 754.46 μmol), Pd(dppf)Cl₂ (18.40 mg, 25.15 μmol) in dioxane (2 mL) and H₂O (0.2 mL) was degassed with N₂, and then the mixture was stirred at 100° C. for 12 h under N₂ atmosphere. The reaction mixture was diluted with water (10 mL), and then extracted with ethyl acetate (3×10 mL). The combined organic layer was washed with brine (3×10 mL), dried over Na₂SO₄, filtered, and concentrated. The residue was purified by prep-TLC (SiO₂, Petroleum ether/Ethyl acetate=10/1 to 5/1) and the pure fraction was concentrated under reduced pressure to give tert-butyl (3R)-3-[[3-[(E)-4-[tert-butyl(dimethyl)silyl]oxybut-1-enyl]-2-pyridyl]-[2-fluoro-4-(triazolo[4,5-b]pyridin-3-yl)benzoyl]amino]piperidine-1-carboxylate (200 mg, 55%) as a yellow oil. LC-MS: [M+H⁺]=702.6.

### Step 2:

A mixture of tert-butyl (3R)-3-[[3-[(E)-4-[tert-butyl(dimethyl)silyl]oxybut-1-enyl]-2-pyridyl]-[2-fluoro-4-(triazolo[4,5-b]pyridin-3-yl)benzoyl]amino]piperidine-1-carboxylate (200 mg, 199.46 μmol) in a solution of 4M HCl in EtOAc (5 mL) was stirred at room temperature for 1 h. The reaction mixture was concentrated. The residue was then purified by prep-HPLC (column: Waters Xbridge BEH C18 100*30 mm*10 μm; mobile phase: [H₂O (10 mM NH₄HCO₃)-MeCN]; gradient: 20%-50% B over 8.0 min) and the combined pure fraction was lyophilized to afford the title compound (18 mg, 18.28%) as a white solid. LC-MS: [M+H⁺]=488.3. ¹H NMR (400 MHZ, DMSO-d₆) δ 0.99-1.28 (m, 1H), 1.39-1.70 (m, 2H), 1.72-2.07 (m, 2H), 2.14-2.34 (m, 2H), 2.37-2.44 (m, 2H), 2.74-3.07 (m, 2H), 3.39-3.63 (m, 2H), 4.32-4.58 (m, 1H), 4.58-4.72 (m, 1H), 6.18-6.37 (m, 1H), 6.38-6.62 (m, 1H), 7.18-7.48 (m, 2H), 7.58-7.79 (m, 1H), 7.85-7.99 (m, 2H), 8.00-8.18 (m, 1H), 8.33-8.50 (m, 1H), 8.67-8.81 (m, 1H), 8.82-8.98 (m, 1H).

### Synthesis of 2-fluoro-N-[3-(4-hydroxybut-1-ynyl)-2-pyridyl]-N-[(3R)-3-piperidyl]-4-(triazolo[4,5-b]pyridin-3-yl)benzamide (Example 105)

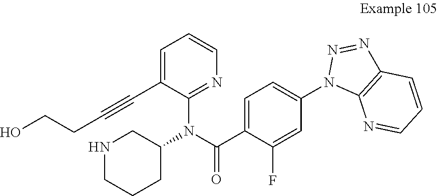 [SMILES: O=C(c1ccc(-n2nnc3cccnc32)cc1F)N(c1ncccc1C#CCCO)[C@@H]1CCCNC1]

Example 105 was prepared according to the procedure described for Example 107 by substituting tert-butyl-dimethyl-[(E)-4-(4,4,5,5-tetramethyl-1,3,2-dioxaborolan-2-yl)but-3-enoxy]silane with 2-methylbut-3-yn-1-ol, provided the title compound as an off-white solid. LC-MS: [M+H⁺]=486.3. ¹H NMR (400 MHZ, DMSO-d₆): δ 8.87 (br d, J=4.1 Hz, 1H), 8.71 (br d, J=8.1 Hz, 1H), 8.48 (br s, 1H), 8.08 (br d, J=10.3 Hz, 1H), 8.01-7.88 (m, 1H), 7.73 (br d, J=2.0 Hz, 1H), 7.63 (dd, J=4.4, 8.2 Hz, 1H), 7.55-7.40 (m, 1H), 7.34-7.22 (m, 1H), 4.98 (br d, J=4.4 Hz, 1H), 4.63-4.37 (m, 1H), 3.65 (br d, J=4.0 Hz, 2H), 2.95-2.75 (m, 2H), 2.69-2.57 (m, 3H), 2.41-2.10 (m, 3H), 1.94-1.75 (m, 1H), 1.68-1.38 (m, 2H), 1.32-1.08 (m, 1H).

### Synthesis of (R)—N-(6-(1H-pyrazol-4-yl)isoquinolin-1-yl)-4-(3H-[1,2,3]triazolo[4,5-b]pyridin-3-yl)-2-fluoro-N-(piperidin-3-yl)benzamide (Example 106)

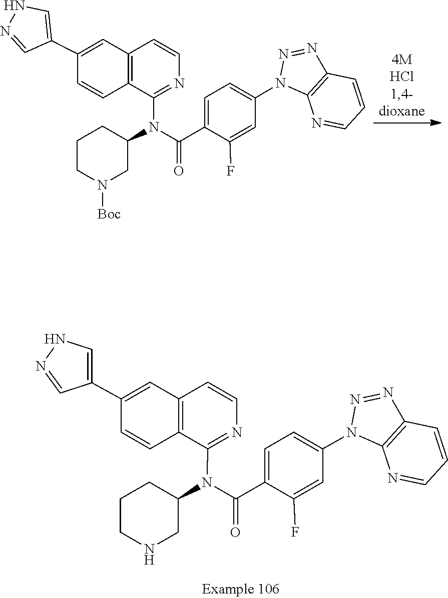 [SMILES: CC(C)(C)OC(=O)N1CCC[C@@H](N(C(=O)c2ccc(-n3nnc4cccnc43)cc2F)c2nccc3cc(-c4cn[nH]c4)ccc23)C1 ; O=C(c1ccc(-n2nnc3cccnc32)cc1F)N(c1nccc2cc(-c3cn[nH]c3)ccc12)[C@@H]1CCCNC1]

To a solution of tert-butyl (R)-3-(N-(6-(1H-pyrazol-4-yl)isoquinolin-1-yl)-4-(3H-[1,2,3]triazolo[4,5-b]pyridin-3-yl)-2-fluorobenzamido)piperidine-1-carboxylate (35 mg, 0.047 mmol) in DCM (1 mL) at 0° C. was added 4M HCl in 1,4-dioxane (2 mL). The reaction was warmed to room temperature and stirred for 2 h. The reaction mixture was concentrated under reduced pressure and dried by lyophilization to afford (R)—N-(6-(1H-pyrazol-4-yl)isoquinolin-1-yl)-4-(3H-[1,2,3]triazolo[4,5-b]pyridin-3-yl)-2-fluoro-N-(piperidin-3-yl)benzamide hydrochloride salt (29 mg) as a white solid. LC-MS: [M+H⁺]=534.25. ¹H NMR (CD₃OD, 400 MHZ): δ 8.72 (d, J=4.8 Hz, 1H), 8.47 (d, J=8.4 Hz, 1H), 8.42-8.37 (m, 1H), 8.17-8.14 (m, 3H), 8.06-7.99 (m, 3H), 7.95-7.90 (m, 1H), 7.73 (d, J=5.6 Hz, 1H), 7.51-7.48 (m, 1H), 7.43-7.32 (m, 1H), 5.31-5.20 (m, 1H), 3.98-3.89 (m, 1H), 3.72-3.66 (m, 1H), 2.93-2.83 (m, 1H), 2.11-2.03 (m, 1H), 1.94-1.90 (m, 2H), 1.36-1.28 (m, 1H), 0.89-0.82 (m, 1H).

### Synthesis of (R)-4-(3H-[1,2,3]triazolo[4,5-b]pyridin-3-yl)-N-(3-ethynylpyridin-2-yl)-2-fluoro-N-(piperidin-3-yl)benzamide (Example 107)

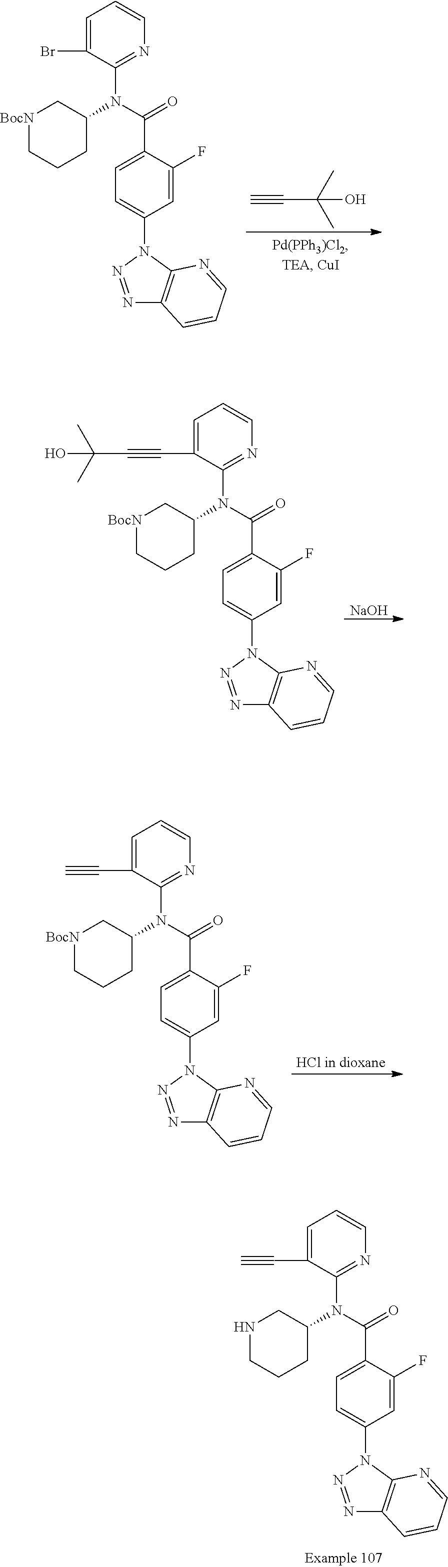

### Step 1:

To a solution of (R)-tert-butyl 3-(4-(3H-[1,2,3]triazolo[4,5-b]pyridin-3-yl)-N-(3-bromopyridin-2-yl)-2-fluorobenzamido)piperidine-1-carboxylate (200 mg, 302 μmol) and 2-methylbut-3-yn-2-ol (295 μL, 3.02 mmol) in DMAc (3 mL) was added Pd(PPh₃)₂Cl₂ (21 mg, 30 μmol), CuI (5.75 mg, 30 μmol), and TEA (126 μL, 905 μmol) at 20° C. under N₂ atmosphere. The mixture was stirred at 120° C. for 12 h before it was allowed to cool and poured into water (20 mL). The mixture was extracted with ethyl acetate (3×30 mL), and the combined organic phase was washed with brine (30 mL), dried over anhydrous Na₂SO₄, and concentrated. The residue was purified by prep-HPLC as for Example 4 and the combined pure fraction was concentrated to give the title compound (60 mg, 23%) as a pale yellow oil.

### Step 2:

To a solution of (R)-tert-butyl 3-(4-(3H-[1,2,3]triazolo[4,5-b]pyridin-3-yl)-2-fluoro-N-(3-(3-hydroxy-3-methylbut-1-yn-1-yl)pyridin-2-yl)benzamido)piperidine-1-carboxylate (30 mg, 45.03 μmol) in toluene (2 mL) at 20° C. was added NaOH (5.40 mg, 135 μmol). The mixture was stirred at 120° C. for 5 h before it was allowed to cool. The reaction mixture was concentrated. The residue was purified by prep-HPLC (column: Phenomenex Luna C18 75*30 mm*3 μm; mobile phase: [H₂O (0.04% HCl)-MeCN]; gradient: 45%-70% B over 8.0 min) and concentrated to give (R)-tert-butyl 3-(4-(3H-[1,2,3]triazolo[4,5-b]pyridin-3-yl)-N-(3-ethynylpyridin-2-yl)-2-fluorobenzamido)piperidine-1-carboxylate (20 mg, 36%) as a pale yellowish oil.

### Step 3:

A solution of (R)-tert-butyl 3-(4-(3H-[1,2,3]triazolo[4,5-b]pyridin-3-yl)-N-(3-ethynylpyridin-2-yl)-2-fluorobenzamido)piperidine-1-carboxylate (15 mg, 25 μmol) in 4M HCl in dioxane (2 mL) was stirred at 20° C. for 2 h. The reaction mixture was concentrated and lyophilized to give the title compound hydrochloride salt (7.5 mg, 43%) as an off-white solid. LC-MS: [M+H⁺]=442.3. ¹H NMR (400 MHz, DMSO-d₆): δ 9.20-8.90 (m, 2H), 8.87 (br d, J=3.7 Hz, 1H), 8.73 (d, J=8.2 Hz, 1H), 8.66-8.54 (m, 1H), 8.14 (br d, J=10.4 Hz, 1H), 8.06-7.98 (m, 1H), 7.94-7.86 (m, 1H), 7.69-7.62 (m, 1H), 7.47-7.36 (m, 2H), 5.10-4.96 (m, 1H), 4.90-4.75 (m, 1H), 3.68-3.47 (m, 2H), 3.22 (br d, J=12.1 Hz, 1H), 2.83-2.65 (m, 2H), 2.03-1.70 (m, 3H), 1.41-1.26 (m, 1H).

### Synthesis of (R)-4-(3H-[1,2,3]triazolo[4,5-b]pyridin-3-yl)-2-fluoro-N-(piperidin-3-yl)-N-(3-(prop-1-en-2-yl)pyridin-2-yl)benzamide (Example 108)

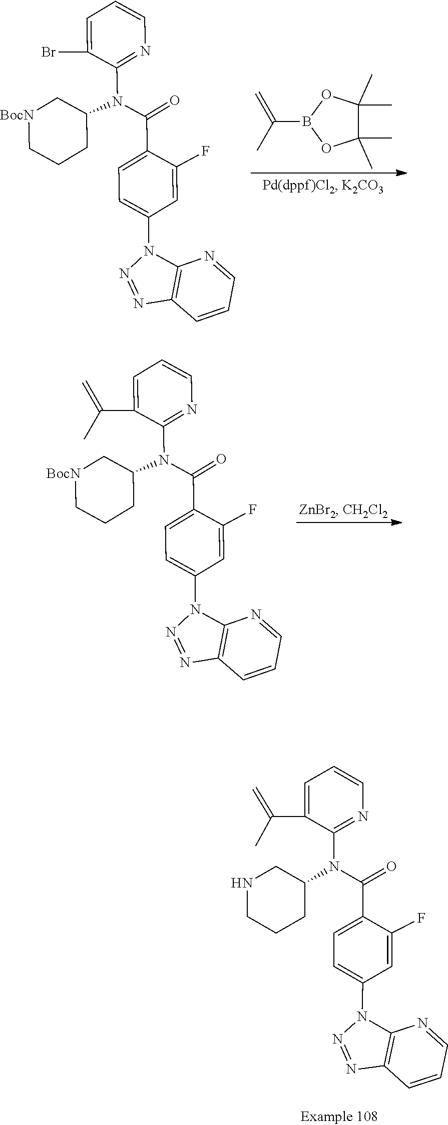 [SMILES: CC(C)(C)OC(=O)N1CCC[C@@H](N(C(=O)c2ccc(-n3nnc4cccnc43)cc2F)c2ncccc2Br)C1 ; C=C(C)B1OC(C)(C)C(C)(C)O1 ; C=C(C)c1cccnc1N(C(=O)c1ccc(-n2nnc3cccnc32)cc1F)[C@@H]1CCCN(C(=O)OC(C)(C)C)C1 ; C=C(C)c1cccnc1N(C(=O)c1ccc(-n2nnc3cccnc32)cc1F)[C@@H]1CCCNC1]

Example 108 was prepared according to the procedure described for Example 104 by substituting tert-butyl-dimethyl-[(E)-4-(4,4,5,5-tetramethyl-1,3,2-dioxaborolan-2-yl)but-3-enoxy]silane with 4,4,5,5-tetramethyl-2-(prop-1-en-2-yl)-1,3,2-dioxaborolane, provided the title compound as an off-white solid. LC-MS: [M+H⁺]=458.2. ¹H NMR (400 MHz, CD₃OD): δ 8.97-8.73 (m, 1H), 8.61-8.43 (m, 2H), 8.15 (br dd, J=1.3, 11.1 Hz, 1H), 8.01 (br d, J=8.2 Hz, 1H), 7.68-7.47 (m, 2H), 7.38-7.14 (m, 2H), 5.41-5.22 (m, 1H), 4.73-4.42 (m, 2H), 3.61-3.36 (m, 1H), 3.12-2.68 (m, 2H), 2.59-2.39 (m, 1H), 2.19 (br s, 1H), 2.00-1.83 (m, 3H), 1.81-1.52 (m, 3H).

### Synthesis of (R,E)-4-(1-methyl-1H-1,2,3-triazol-4-yl)-N-(piperidin-3-yl)-N-(4-styrylpyridin-2-yl)benzamide (Example 109)

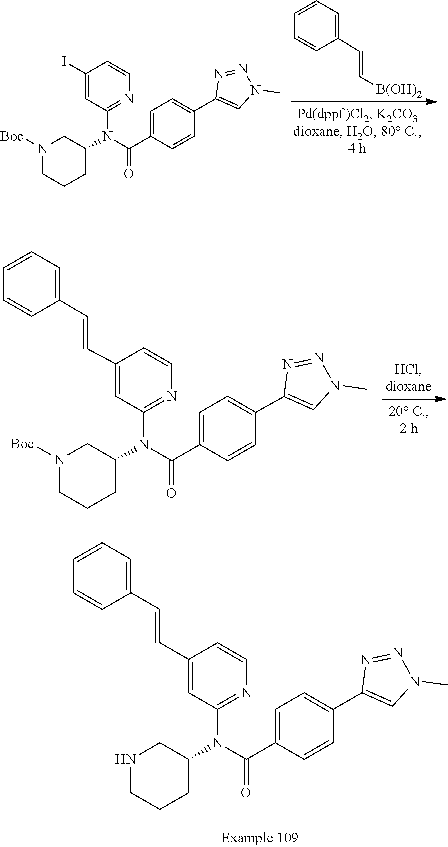

Example 109 was prepared according to the procedure described for Example 110 by substituting 1H-pyrazol-3-ylboronic acid with (E)-styrylboronic acid, provided the title compound as an off-white solid. LC-MS: [M+H⁺]=465.4. ¹H NMR (400 MHZ, CD₃OD): δ 8.46 (d, J=5.4 Hz, 1H), 8.33 (s, 1H), 7.73 (d, J=8.1 Hz, 2H), 7.70-7.64 (m, 2H), 7.63-7.52 (m, 3H), 7.48 (d, J=8.1 Hz, 2H), 7.42-7.31 (m, 3H), 7.24-7.09 (m, 1H), 5.00-4.93 (m, 1H), 4.16-4.09 (m, 3H), 3.81 (br d, J=9.7 Hz, 1H), 3.63-3.47 (m, 2H), 3.38 (br d, J=12.7 Hz, 1H), 2.94 (br t, J=12.3 Hz, 1H), 2.19 (br d, J=10.0 Hz, 1H), 2.14-2.04 (m, 1H), 1.99-1.80 (m, 2H).

### Synthesis of 4-(1-methyltriazol-4-yl)-N-[(3R)-3-piperidyl]-N-[4-(1H-pyrazol-3-yl)-2-pyridyl] benzamide (Example 110)

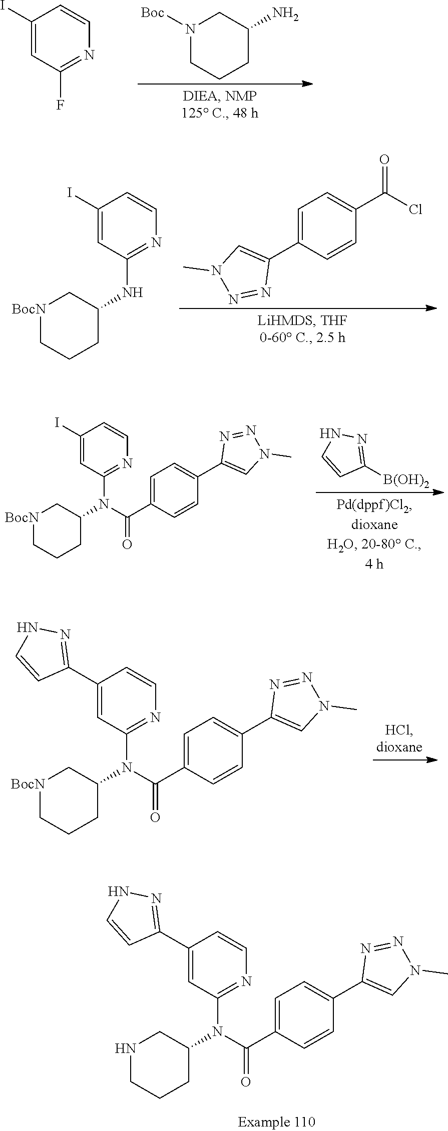

### Step 1:

To a solution of tert-butyl (3R)-3-aminopiperidine-1-carboxylate (10 g, 49.93 mmol) in NMP (60 mL) at 20° C. was added DIEA (32.27 g, 249.65 mmol, 43.49 mL) and 2-fluoro-4-iodo-pyridine (13.36 g, 59.92 mmol). The mixture was heated to 125° C. and stirred for 48 h. The reaction mixture was poured into water (500 mL) and was extracted with ethyl acetate (3×100 mL). The combined organic phase was washed with brine (200 mL), dried over anhydrous Na₂SO₄, and concentrated. The residue was purified by flash column chromatography (SiO₂, Petroleum ether/ethyl acetate=10/1 to 9/1) to give tert-butyl (3R)-3-[(4-iodo-2-pyridyl)amino]piperidine-1-carboxylate (8 g, 37%) as a gray solid. LC-MS: [M+H⁺]=404.2.

### Step 2:

A solution of 4-(1-methyltriazol-4-yl)benzoic acid (3 g, 14.76 mmol) and SOCl₂ (5 mL) was stirred at 80° C. for 2 h under N₂atmosphere. The reaction mixture was concentrated to give crude 4-(1-methyltriazol-4-yl)benzoyl chloride (3.2 g, 75%) as a white solid, which was used in the next step directly.

### Step 3:

A mixture of tert-butyl (3R)-3-[(4-iodo-2-pyridyl)amino]piperidine-1-carboxylate (3.40 g, 8.43 mmol) in THF (40 mL) at 0° C. was degassed and purged with N₂ thrice, and then followed by addition of DIEA (7.26 g, 56.17 mmol, 9.78 mL). The mixture was stirred for 30 min and followed by addition of 4-(1-methyltriazol-4-yl)benzoyl chloride (2.49 g, 11.23 mmol). The reaction was heated at 60° C. for 2 h under N₂ atmosphere before it was concentrated, and the residue was diluted with water (100 mL) and extracted with ethyl acetate (3×70 mL). The combined organic layer was washed with brine (200 mL), dried over Na₂SO₄, filtered, and concentrated. The residue was purified by silica gel chromatography (petroleum ether:ethyl acetate=4:1) to give tert-butyl (3R)-3-[(4-iodo-2-pyridyl)-[4-(1-methyltriazol-4-yl)benzoyl]amino]piperidine-1-carboxylate (3.5 g, 46%) as an off-white solid. LC-MS: [M+H⁺]=589.3.

### Step 4:

To a solution of tert-butyl (3R)-3-[(4-iodo-2-pyridyl)-[4-(1-methyltriazol-4-yl)benzoyl]amino]piperidine-1-carboxylate (60 mg, 101.96 μmol) and 1H-pyrazol-3-ylboronic acid (12.55 mg, 112.16 μmol) in dioxane (0.5 mL) and H₂O (0.1 mL) was added Pd(dppf)Cl₂ (7.46 mg, 10.20 μmol) and K₂CO₃ (42.28 mg, 305.89 μmol) under N₂. The mixture was stirred at 90° C. for 4 h before it was cooled to 25° C. The reaction mixture was filtered, and the filtrate was concentrated. The residue was purified by Prep-HPLC. (column: Phenomenex Luna C18 75*30 mm*3 μm; mobile phase: [H₂O (0.04% HCl)-MeCN]; gradient: 20%-50% B over 8.0 min) to give tert-butyl (3R)-3-[[4-(1-methyltriazol-4-yl)benzoyl]-[4-(1H-pyrazol-3-yl)-2-pyridyl]amino]piperidine-1-carboxylate (35 mg, 61%) as a white solid. LC-MS: [M+H⁺]=529.4.

### Step 5:

To a solution of tert-butyl (3R)-3-[4-(1-methyltriazol-4-yl)benzoyl]-[4-(1H-pyrazol-3-yl)-2-pyridyl]amino]piperidine-1-carboxylate (35 mg, 66.21 μmol) in dioxane (1 mL) was added a solution of 4M HCl in dioxane (16.55 μL). The mixture was stirred at 25° C. for 1 h. The reaction mixture was concentrated to give 4-(1-methyltriazol-4-yl)-N-[(3R)-3-piperidyl]-N-[4-(1H-pyrazol-3-yl)-2-pyridyl] benzamide (14.9 mg, 49%) as a white solid. LC-MS: [M+H⁺]=429.3. ¹H NMR (400 MHZ, CD₃OD): δ 8.55 (d, J=5.5 Hz, 1H), 8.38 (s, 1H), 7.94-7.86 (m, 2H), 7.81 (d, J=2.3 Hz, 1H), 7.71 (d, J=8.3 Hz, 2H), 7.49 (d, J=8.3 Hz, 2H), 6.92 (d, J=2.4 Hz, 1H), 5.03-4.92 (m, 1H), 4.15 (s, 3H), 3.87-3.77 (m, 1H), 3.60 (s, 1H), 3.56-3.46 (m, 1H), 3.37 (br d, J=12.8 Hz, 1H), 2.98-2.88 (m, 1H), 2.20 (br d, J=10.6 Hz, 1H), 2.13-2.04 (m, 1H), 1.99-1.80 (m, 2H).

### Synthesis of (R)-4-(1-methyl-1H-1,2,3-triazol-4-yl)-N-(8-methylisoquinolin-1-yl)-N-(piperidin-3-yl) benzamide (Example 111)

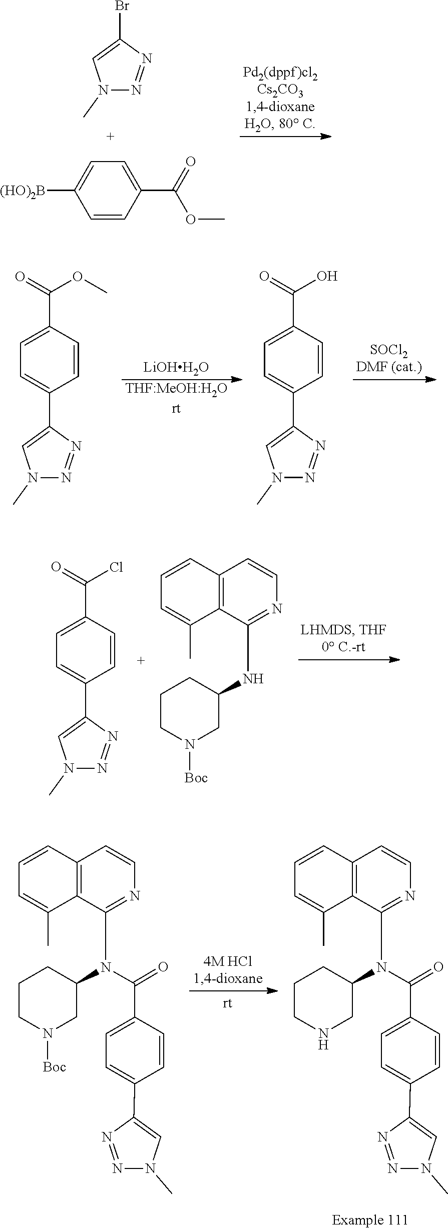

### Step 1

To a solution of 4-bromo-1-methyl-1H-1,2,3-triazole (5 g, 30.86 mmol) in 1,4-dioxane (100 mL) was added Cs₂CO₃ (30.1 g, 92.58 mmol) and 4-(methoxycarbonyl)phenyl)-boronic acid (10 g, 55.55 mmol) at room temperature. The reaction mixture was degassed with nitrogen for 10 min and followed by addition of Pd₂(dppf)Cl₂ (1.26 g, 1.543 mmol) which was again degassed with nitrogen for 5 min. The reaction mixture was then stirred at 80° C. for 16 h. The reaction mixture was cooled and filtered through celite and washed with EtOAc, and the filtrate was concentrated. The crude residue was diluted with water (100 mL) and extracted with EtOAc (500 mL). The organic layer was washed with brine, dried over Na₂SO₄, and concentrated. The crude product was purified by Combi-flash using 5% MeOH in DCM to affords methyl 4-(1-methyl-1H-1,2,3-triazol-4-yl)benzoate (5.4 g, 80%). LC-MS: [M+H⁺]=218.1.

### Step 2:

To a solution of methyl 4-(1-methyl-1H-1,2,3-triazol-4-yl)benzoate (3.5 g, 16.11 mmol) in a mixture of THF, MeOH, and water (v:v:v=15:8:8, 21 mL) at room temperature was added LiOH monohydrate (2.01 g, 48.33 mmol). The reaction mixture was stirred for 3 h, after which the reaction was concentrated. The crude residue was dissolved in water (10 mL) and extracted with EtOAc (30 mL). The aqueous layer was acidified with 1N HCl (20 mL). The precipitate was collected by filtration and dried to afford 4-(1-methyl-1H-1,2,3-triazol-4-yl) benzoic acid (2.9 g, 90%) as an off-white solid. LC-MS: [M+H⁺]=204.05.

### Step 3:

To a solution of 4-(1-methyl-1H-1,2,3-triazol-4-yl) benzoic acid (300 mg, 1.47 mmol) in SOCl₂ (15 mL) was added a catalytic amount of DMF (0.2 mL) at room temperature. The reaction was then stirred at 100° C. for 16 h. The reaction mixture was concentrated under reduced pressure and azeotroped with toluene to afford crude 4-(1-methyl-1H-1,2,3-triazol-4-yl) benzoyl chloride (320 mg) as an off-white solid, which was used in the next step directly.

### Step 4:

To a solution of tert-butyl (R)-3-((8-methylisoquinolin-1-yl)amino)piperidine-1-carboxylate (200 mg, 0.585 mmol) in THF (5 mL) at 0° C. was added LiHMDS (1.17 mL, 1.755 mmol). The reaction mixture was stirred for 10 min and followed by addition of a solution of 4-(1-methyl-1H-1,2,3-triazol-4-yl) benzoyl chloride (155 mg, 0.702 mmol) in THF (5 mL). The reaction was warmed to room temperature and stirred for 50 min before it was diluted with water (30 mL) and extracted with ethyl acetate (2×100 mL). The combined organic layer was washed with brine, dried over Na₂SO₄, and concentrated. The crude product was purified by Combi-flash using 50% EtoAc in hexane and was further purified by prep-HPLC (Conditions: X-SELECT; Mobile phase A: 0.1% formic acid in water (100%), Mobile phase B: 100% MeCN, Gradient: Time (min) % B: 0/50, 10/80; Flow: 15 mL/min, Dilution: MeCN+H₂O+THF). The pure fractions were combined and dried by lyophilization to afford tert-butyl (R)-3-(4-(1-methyl-1H-1,2,3-triazol-4-yl)-N-(8-methylisoquinolin-1-yl) benzamido)piperidine-1-carboxylate (50 mg, 16%) as an off-white solid. LC-MS: [M+H⁺]=527.30.

### Step 5:

tert-Butyl (R)-3-(4-(1-methyl-1H-1,2,3-triazol-4-yl)-N-(8-methylisoquinolin-1-yl)benzamido)piperidine-1-carboxylate (50 mg, 0.094 mmol) at 0° C. was added to a solution of 4M HCl in 1,4-dioxane (5 mL). The reaction was warmed to room temperature and stirred for 2 h. The reaction mixture was concentrated under reduced pressure and dried by lyophilization to afford the title compound hydrochloride salt (40 mg, 93%) as an off-white solid. LC-MS: [M+H⁺]=427.25. ¹H NMR (CD₃OD, 400 MHZ): δ 8.51-8.46 (m, 1H), 8.23 (s, 1H), 7.83-7.79 (m, 2H), 7.69-7.65 (m, 1H), 7.49-7.46 (m, 1H), 7.43-7.37 (m, 3H), 7.12 (d, J=8.0 Hz, 1H), 5.13-5.06 (m, 1H), 4.09 (s, 3H), 3.97-3.94 (m, 1H), 3.84-3.82 (m, 1H), 3.45-3.37 (m, 1H), 3.02-2.96 (m, 1H), 2.75 (s, 3H), 2.69-2.65 (m, 1H), 1.95-1.81 (m, 2H), 1.51-1.47 (m, 1H).

### Synthesis of (R)-4-(3H-[1,2,3]triazolo[4,5-b]pyridin-3-yl)-2-fluoro-N-(3-(1-methyl-1H-imidazol-4-yl)pyridin-2-yl)-N-(piperidin-3-yl)benzamide (Example 112)

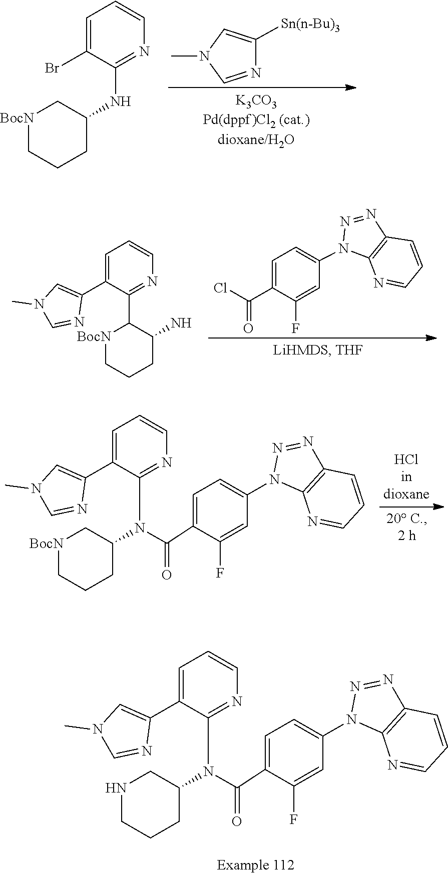

To a solution of tributyl-(1-methylimidazol-4-yl)stannane (104.18 mg, 280.70 μmol) and tert-butyl (R)-3-((3-bromopyridin-2-yl)amino)piperidine-1-carboxylate (0.1 g, 280.70 μmol) in dioxane (1 mL) and H₂O (0.1 mL) at 20° C. under N₂ atmosphere was added K₂CO₃ (116.38 mg, 842.09 μmol) and Pd(dppf)Cl₂ (8.22 mg, 11.23 μmol). The mixture was stirred at 100° C. for 12 h under N₂ atmosphere before it was cooled and filtered. The filtrate was diluted with water (5 mL) and extracted with ethyl acetate (3×5 mL). The combined organic layer was washed with brine (6×5 mL), dried over Na₂SO₄, filtered, and concentrated. The residue was purified by prep-TLC (SiO₂, Petroleum ether/Ethyl acetate=1/2) to give tert-butyl (R)-3-((3-(1-methyl-1H-imidazol-4-yl)pyridin-2-yl)amino)piperidine-1-carboxylate (0.05 g, 44%) as an off-white solid.

### Step 2:

To a solution of tert-butyl (R)-3-((3-(1-methyl-1H-imidazol-4-yl)pyridin-2-yl)amino)piperidine-1-carboxylate (0.05 g, 125.89 μmol, 1 eq) in THF (0.5 mL) at 0° C. under N₂ atmosphere was added LiHMDS in THF (1 M, 188.84 μL). The mixture was stirred at 0° C. for 30 minutes under N₂ and followed by dropwise addition of 2-fluoro-4-(triazolo[4,5-b]pyridin-3-yl)benzoyl chloride (58.05 mg, 188.84 μmol) in THF (0.5 mL). The mixture was stirred at 20° C. for 12 h under N₂ before it was quenched with saturated aq. NH₄Cl (5 mL), and then the mixture was extracted with ethyl acetate (3×5 mL). The combined organic phase was washed with brine (2×5 mL), dried over Na₂SO₄, filtered, and concentrated. The residue was purified by prep-TLC (SiO₂, Petroleum ether/Ethyl acetate=1/2) to give crude tert-butyl (R)-3-(4-(3H-[1,2,3]triazolo[4,5-b]pyridin-3-yl)-2-fluoro-N-(3-(1-methyl-1H-imidazol-4-yl)pyridin-2-yl)benzamido)piperidine-1-carboxylate (0.02 g) as an orange oil.

### Step 3:

To a solution of tert-butyl (R)-3-(4-(3H-[1,2,3]triazolo[4,5-b]pyridin-3-yl)-2-fluoro-N-(3-(1-methyl-1H-imidazol-4-yl)pyridin-2-yl)benzamido)piperidine-1-carboxylate (0.02 g, 33.46 μmol) in a solution of 4M HCl in dioxane (1 mL) at 20° C. under N₂ was stirred for 2 h The reaction mixture was concentrated under reduced pressure. The residue was purified by prep-HPLC (column: Phenomenex Luna C18 75*30 mm*3 μm; mobile phase: [H₂O (0.04% HCl)-MeCN]; gradient: 1%-25% B over 8.0 min) and lyophilized to give (R)-4-(3H-[1,2,3]triazolo[4,5-b]pyridin-3-yl)-2-fluoro-N-(3-(1-methyl-1H-imidazol-4-yl)pyridin-2-yl)-N-(piperidin-3-yl)benzamide hydrochloride salt (5 mg, 28%) as an off-white solid. LC-MS: [M+H⁺]=498.30. ¹H NMR (400 MHZ, DMSO-d₆): δ 9.54-9.16 (m, 1H), 8.87 (br d, J=3.3 Hz, 1H), 8.73 (br d, J=8.4 Hz, 1H), 8.64-8.31 (m, 2H), 8.07-7.89 (m, 3H), 7.78-7.59 (m, 2H), 7.54-7.37 (m, 1H), 7.15-6.90 (m, 1H), 6.91-6.85 (m, 1H), 5.03 (br t, J=11.3 Hz, 1H), 3.96-3.83 (m, 3H), 1.91-1.70 (m, 3H), 1.35-1.18 (m, 1H).

### Synthesis of (R)-4-(3H-[1,2,3]triazolo[4,5-b]pyridin-3-yl)-N-(3-(2,5-dihydro-1H-pyrrol-3-yl)pyridin-2-yl)-2-fluoro-N-(piperidin-3-yl)benzamide (Example 113)

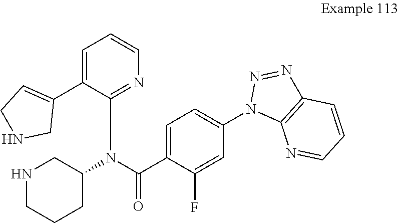 [SMILES: O=C(c1ccc(-n2nnc3cccnc32)cc1F)N(c1ncccc1C1=CCNC1)[C@@H]1CCCNC1]

Example 113 was prepared according to the procedure shown for Example 104 by substituting tert-butyl-dimethyl-[(E)-4-(4,4,5,5-tetramethyl-1,3,2-dioxaborolan-2-yl)but-3-enoxy]silane with tert-butyl 3-(4,4,5,5-tetramethyl-1,3,2-dioxaborolan-2-yl)-2,5-dihydro-1H-pyrrole-1-carboxylate providing the title compound hydrochloride salt (10 mg) as a white solid. LC-MS: [M+H⁺]=485.4. ¹H NMR (400 MHZ, DMSO-d₆): δ 10.10 (br s, 2H), 9.45 (br d, J=8.4 Hz, 1H), 9.28-9.13 (m, 1H), 8.88 (dd, J=1.3, 4.6 Hz, 1H), 8.73 (dd, J=1.3, 8.3 Hz, 1H), 8.57-8.50 (m, 1H), 8.14-8.06 (m, 2H), 7.75-7.60 (m, 2H), 7.46-7.36 (m, 2H), 6.29-6.19 (m, 1H), 4.95-4.68 (m, 1H), 4.44-4.15 (m, 3H), 4.00-3.77 (m, 1H), 3.71-3.52 (m, 2H), 3.25 (br d, J=11.3 Hz, 1H), 3.08-2.72 (m, 1H), 1.96-1.71 (m, 3H), 1.51-1.34 (m, 1H).

### Synthesis of (R)-4-(3H-[1,2,3]triazolo[4,5-b]pyridin-3-yl)-2-fluoro-N-(3-(isoxazol-4-yl)pyridin-2-yl)-N-(piperidin-3-yl)benzamide (Example 114)

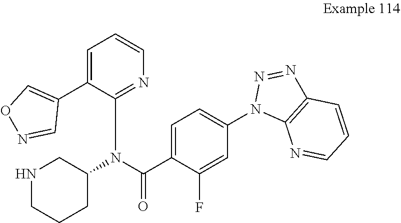 [SMILES: O=C(c1ccc(-n2nnc3cccnc32)cc1F)N(c1ncccc1-c1cnoc1)[C@@H]1CCCNC1]

Example 114 was prepared according to the procedure described for Example 104 by substituting tert-butyl-dimethyl-[(E)-4-(4,4,5,5-tetramethyl-1,3,2-dioxaborolan-2-yl)but-3-enoxy]silane with 4-(4,4,5,5-tetramethyl-1,3,2-dioxaborolan-2-yl)isoxazole, provided the title compound as a white solid. LC-MS: [M+H⁺]=485.3. ¹H NMR (400 MHZ, DMSO-d₆): δ 1.19-1.42 (m, 1H), 1.69-1.94 (m, 3H), 2.71-2.91 (m, 1H), 3.26 (br d, J=12.13 Hz, 1H), 3.68 (br s, 2H), 4.90-5.25 (m, 1H), 6.93 (t, J=8.07 Hz, 1H), 7.44-7.53 (m, 1H), 7.65 (dd, J=8.38, 4.50 Hz, 1H), 7.96 (ddd, J=13.54, 8.16, 1.81 Hz, 2H), 8.05 (dd, J=11.01, 1.88 Hz, 1H), 8.62 (dd, J=4.69, 1.69 Hz, 1H), 8.74 (dd, J=8.38, 1.25 Hz, 1H), 8.85-8.91 (m, 3H), 8.96-9.07 (m, 1H), 9.21-9.29 (m, 1H), 9.30 (s, 1H).

### Synthesis of (R)-4-(3H-[1,2,3]triazolo[4,5-b]pyridin-3-yl)-2-fluoro-N-(3-(1-methyl-1H-pyrazol-4-yl)pyridin-2-yl)-N-(piperidin-3-yl)benzamide (Example 115)

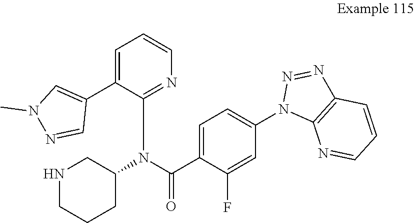 [SMILES: Cn1cc(-c2cccnc2N(C(=O)c2ccc(-n3nnc4cccnc43)cc2F)[C@@H]2CCCNC2)cn1]

Example 115 was prepared according to the procedure described for Example 104 by substituting tert-butyl-dimethyl-[(E)-4-(4,4,5,5-tetramethyl-1,3,2-dioxaborolan-2-yl)but-3-enoxy]silane with 1-methyl-4-(4,4,5,5-tetramethyl-1,3,2-dioxaborolan-2-yl)-1H-pyrazole, providing the title compound hydrochloride salt as an off-white solid. LC-MS: [M+H⁺]=498.3. ¹H NMR (400 MHZ, CD₃OD): δ 8.81 (dd, 1H, J=1.3, 4.5 Hz), 8.5-8.6 (m, 2H), 7.9-8.1 (m, 2H), 7.8-7.8 (m, 2H), 7.5-7.6 (m, 2H), 7.36 (dd, 1H, J=4.8, 7.9 Hz), 6.90 (t, 1H, J=7.9 Hz), 5.1-5.3 (m, 1H), 4.00 (s, 3H), 3.8-3.9 (m, 2H), 3.41 (br d, 1H, J=12.6 Hz), 2.9-3.1 (m, 1H), 1.9-2.1 (m, 3H), 1.5-1.6 (m, 1H).

### Synthesis of (R)-4-(3H-[1,2,3]triazolo[4,5-b]pyridin-3-yl)-2-fluoro-N-(3-(1-(2-hydroxyethyl)-1H-pyrazol-4-yl)pyridin-2-yl)-N-(piperidin-3-yl)benzamide (Example 116))

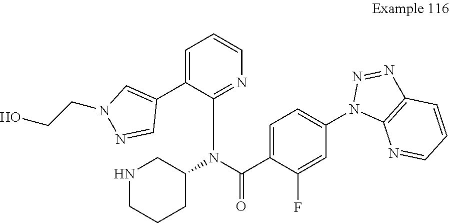 [SMILES: O=C(c1ccc(-n2nnc3cccnc32)cc1F)N(c1ncccc1-c1cnn(CCO)c1)[C@@H]1CCCNC1]

Example 116 was prepared according to the procedure described for Example 104 by substituting tert-butyl-dimethyl-[(E)-4-(4,4,5,5-tetramethyl-1,3,2-dioxaborolan-2-yl)but-3-enoxy]silane with 1-(2-((tert-butyldimethylsilyl)oxy)ethyl)-4-(4,4,5,5-tetramethyl-1,3,2-dioxaborolan-2-yl)-1H-pyrazole, provided the title compound as an off-white solid. LC-MS: [M+H⁺]=528.3. ¹H NMR (400 MHZ, DMSO-d₆): δ 8.85 (dd, J=1.2, 4.4 Hz, 1H), 8.70 (dd, J=1.3, 8.3 Hz, 1H), 8.44-8.36 (m, 1H), 7.96-7.90 (m, 2H), 7.89-7.77 (m, 2H), 7.69-7.59 (m, 2H), 7.30 (dd, J=4.6, 7.9 Hz, 1H), 6.95-6.84 (m, 1H), 5.02-4.93 (m, 1H), 4.67-4.45 (m, 1H), 4.31-4.17 (m, 2H), 3.87-3.78 (m, 2H), 3.40 (br s, 1H), 3.08 (t, J=10.9 Hz, 1H), 2.88-2.80 (m, 1H), 2.36-2.13 (m, 2H), 1.76-1.45 (m, 3H), 1.16-1.06 (m, 1H).

### Synthesis of methyl (R)-1-(4-(3H-[1,2,3]triazolo[4,5-b]pyridin-3-yl)-2-fluoro-N-(piperidin-3-yl)benzamido)isoquinoline-6-carboxylate (Example 117)

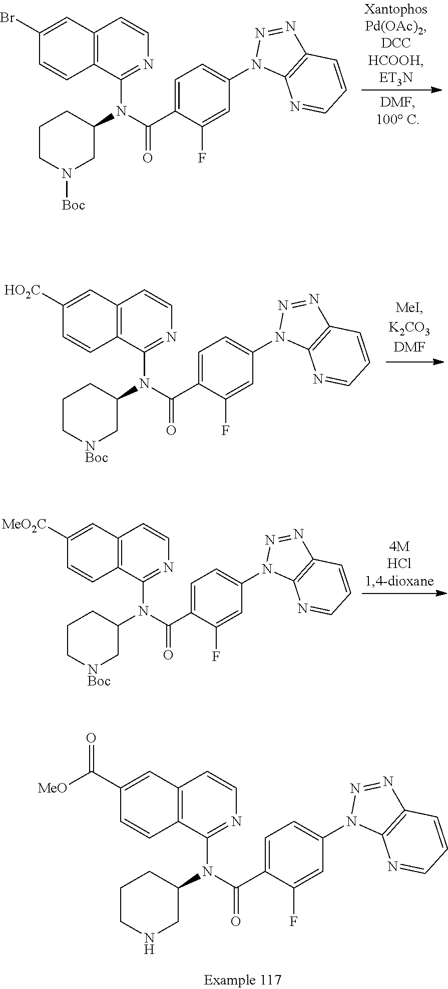

### Step 1:

To a solution of tert-butyl (R)-3-(4-(3H-[1,2,3]triazolo[4,5-b]pyridin-3-yl)-N-(6-bromoisoquinolin-1-yl)-2-fluorobenzamido)piperidine-1-carboxylate (700 mg, 1.085 mmol) in DMF (10 mL) at room temperature were added Xantphos (37.66 mg, 0.0651 mmol), formic acid (324.6 mg, 7.052 mmol), DCC (40.2 mg, 0.0223 mmol), and Et₃N (217.5 mg, 2.17 mmol). The reaction mixture was degassed with nitrogen for 10 min before Pd(OAc)₂ (14.61 mg, 0.0651 mmol) was added. The reaction mixture was stirred at 100° C. for 16 h before it was allowed to cool. The reaction mixture was diluted with cold ice water (20 mL), and the precipitate solid was isolated by filtration and dried to afford (R)-1-(4-(3H-[1,2,3]triazolo[4,5-b]pyridin-3-yl)-N-(1-(tert-butoxycarbonyl)piperidin-3-yl)-2-fluorobenzamido)isoquinoline-6-carboxylic acid (550 mg) as a brown solid. LC-MS: [M−H⁺]=610.25.

### Step 2:

To a solution of (R)-1-(4-(3H-[1,2,3]triazolo[4,5-b]pyridin-3-yl)-N-(1-(tert-butoxycarbonyl)piperidin-3-yl)-2-fluorobenzamido)isoquinoline-6-carboxylic acid (420 mg, 0.687 mmol) in DMF (10 mL) at 0° C. was added K₂CO₃ (284.9 mg, 2.061 mmol). The reaction mixture was stirred at 0° C. for 10 min before iodomethane (117 mg, 0.824 mmol) was added. The reaction mixture was then stirred at room temperature for 16 h before it was diluted with water and extracted with EtOAc (2×100 mL). The combined organic layer was washed with brine, dried over Na₂SO₄, and concentrated. The crude product was purified by Combi-flash using 100% EtOAc in hexane to afford methyl (R)-1-(4-(3H-[1,2,3]triazolo[4,5-b]pyridin-3-yl)-N-(1-(tert-butoxycarbonyl)piperidin-3-yl)-2-fluorobenzamido)isoquinoline-6-carboxylate (230 mg) as a yellow solid. This compound was further purified by prep-HPLC [Conditions: Luna OMEGA C-18 (250×21.2×5 μm), Mobile phase A: 0.1% formic acid in water (100%), Mobile phase B: 100% MeCN, Gradient: T/% B: 1/20, 10/50, Flow: 15 mL/min, Dilution: MeCN/H₂O/THF]. Combined pure fraction was dried by lyophilization to afford methyl (R)-1-(4-(3H-[1,2,3]triazolo[4,5-b]pyridin-3-yl)-N-(1-(tert-butoxycarbonyl)piperidin-3-yl)-2-fluorobenzamido)isoquinoline-6-carboxylate (90 mg, 40%) as an off-white solid. LC-MS: [M+H⁺]=626.65.

### Step 3:

To a solution of methyl (R)-1-(4-(3H-[1,2,3]triazolo[4,5-b]pyridin-3-yl)-N-(1-(tert-butoxycarbonyl)piperidin-3-yl)-2-fluorobenzamido)isoquinoline-6-carboxylate (50 mg, 0.0799 mmol) in DCM at 0° C. was added 4M HCl in 1,4-dioxane (10 mL). The reaction was warmed to room temperature and stirred for 2 h. The reaction mixture was concentrated under reduced pressure and lyophilized to afford the title compound hydrochloride salt (38 mg, 90%) as a white solid. LC-MS: [M+H⁺]=526.20. ¹H NMR (CD₃OD, 400 MHZ): δ 8.88 (d, J=3.6 Hz, 1H), 8.72-8.71 (m, 1H), 8.68-8.66 (m, 1H), 8.64-8.62 (m, 1H), 8.46-8.43 (m, 1H), 8.40-8.38 (m, 1H), 8.19-8.16 (m, 1H), 8.09-8.04 (m, 2H), 7.68-7.64 (m, 1H), 7.48-7.44 (m, 1H), 5.46-5.40 (m, 1H), 4.37-4.13 (m, 1H), 4.09 (s, 3H), 3.88-3.82 (m, 1H), 3.04-2.99 (m, 1H), 2.18-2.05 (m, 1H), 2.02-1.98 (m, 2H), 1.50-1.43 (m, 1H), 1.11-1.04 (m, 1H).

### Synthesis of (R)-1-(4-(3H-[1,2,3]triazolo[4,5-b]pyridin-3-yl)-2-fluoro-N-(piperidin-3-yl)benzamido)isoquinoline-6-carboxylic acid (Example 118))

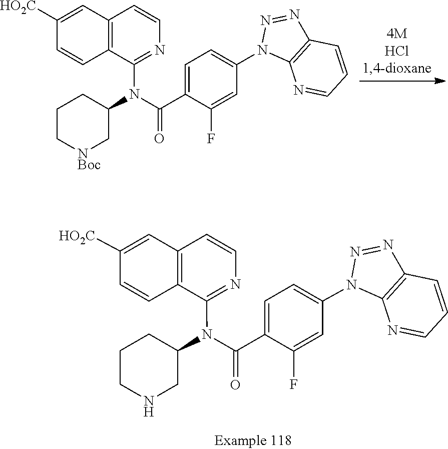 [SMILES: CC(C)(C)OC(=O)N1CCC[C@@H](N(C(=O)c2ccc(-n3nnc4cccnc43)cc2F)c2nccc3cc(C(=O)O)ccc23)C1 ; O=C(O)c1ccc2c(N(C(=O)c3ccc(-n4nnc5cccnc54)cc3F)[C@@H]3CCCNC3)nccc2c1]

To a solution of (R)-1-(4-(3H-[1,2,3]triazolo[4,5-b]pyridin-3-yl)-N-(1-(tert-butoxycarbonyl)piperidin-3-yl)-2-fluorobenzamido)isoquinoline-6-carboxylic acid (100 mg, 0.16 mmol) in DCM (2 mL) at 0° C. was added 4M HCl in 1,4-dioxane (10 mL). The reaction was warmed to room temperature and stirred for 2 h. The reaction mixture was concentrated under reduced pressure, and the crude residue was purified by prep-HPLC (Conditions: Column: Luna OMEGA C-18 (250×21.2×5 μm)), Mobile phase A: 0.1% TFA in H₂O (100%), Mobile phase B: 100% MeCN, Gradient: Time (min)/% B: 0/30, 5/30, 10/50, Flow: 15 mL/min, Dilution: MeCN+H₂O+THF). Pure fractions were combined and lyophilized to afford (R)-1-(4-(3H-[1,2,3]triazolo[4,5-b]pyridin-3-yl)-2-fluoro-N-(piperidin-3-yl)benzamido)isoquinoline-6-carboxylic acid trifluoroacetic acid salt (38 mg) as an off-white solid. LC-MS: [M+H⁺]=512.80. ¹H NMR (CD₃OD, 400 MHZ): δ 8.73 (d, J=4.4 Hz, 1H), 8.56-8.46 (m, 3H), 8.29-8.23 (m, 2H), 8.03 (d, J=11.2 Hz, 1H), 7.95-7.90 (m, 2H), 7.52-7.49 (m, 1H), 7.34-7.30 (m, 1H), 5.31-5.21 (m, 1H), 4.88-4.78 (m, 1H), 3.99-3.96 (m, 1H), 3.74-3.68 (m, 1H), 2.90-2.84 (m, 1H), 1.92-1.90 (m, 1H), 1.87-1.83 (m, 2H), 1.33-1.26 (m, 1H).

### Synthesis of (R)-2-fluoro-N-(1-methyl-1H-pyrazolo[3,4-c]pyridin-7-yl)-N-(piperidin-3-yl)-4-(pyrimidin-2-ylamino)benzamide (Example 119))

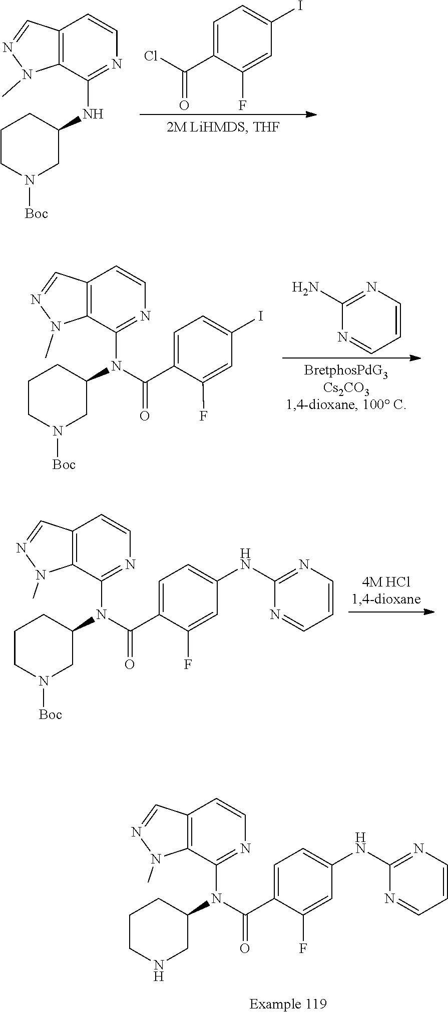

### Step 1:

To a solution of tert-butyl (R)-3-((1-methyl-1H-pyrazolo[3,4-c]pyridin-7-yl)amino)piperidine-1-carboxylate (500 mg, 1.508 mmol) in THF (10 mL) at 0° C. was added 1M LiHMDS in THF (3.2 mL, 4.525 mmol). The reaction was stirred for 15 min before a solution of 2-fluoro-4-iodobenzoyl chloride (510 mg, 1.810 mmol) in THF (5 mL) was added. The reaction mixture was stirred at 0° C. for 10 min. The reaction mixture was quenched with ice cold water and extracted with EtOAc (2×50 mL). The combined organic layer was washed with brine, dried over on Na₂SO₄, and concentrated under reduced pressure. The crude product was purified by Combi-flash using 40% EtOAc in hexane first and then further purified by prep-HPLC [Conditions: KINETEX C-18 (250×21.2×5 μm), Mobile phase A: 0.1% formic acid in water (100%), Mobile phase B: 100% MeCN, Gradient: T/% B: 0/60, 10/80, Flow: 15 mL/min, Dilution: MeCN/H₂O/THF]. Pure fractions were combined and lyophilized to afford tert-butyl (R)-3-(2-fluoro-4-iodo-N-(1-methyl-1H-pyrazolo[3,4-c]pyridin-7-yl)benzamido)piperidine-1-carboxylate (100 mg, 22%) as an off-white solid. LC-MS: [M+H⁺]=580.10.

### Step 2:

To a solution of tert-butyl (R)-3-(2-fluoro-4-iodo-N-(1-methyl-1H-pyrazolo[3,4-c] pyridin-7-yl)benzamido)piperidine-1-carboxylate (80 mg, 0.207 mmol) and pyrimidin-2-amine (59 mg, 0.621 mmol) in 1,4-dioxane (8 mL) were added Cs₂CO₃ (101 mg, 0.310 mmol) and followed by BrettphosPdG₃ (37 mg, 0.0414 mmol) at room temperature. The reaction mixture was purged with nitrogen for 10 min and then stirred at 100° C. for 16 h. The reaction mixture was cooled and poured into water (10 mL) and extracted with EtOAc (2×20 mL). The combined organic layer was washed with brine, dried over Na₂SO₄, and concentrated. The crude product was purified by Combi-flash using 60% EtOAc in hexane first and then was further purified by prep-HPLC [Conditions: X-SELECT CSH, Mobile phase A: 0.1% formic acid in water (100%), Mobile phase B: 100% MeCN, Gradient: T/% B: 0/65, 10/80, Flow: 15 mL/min, Dilution: MeCN+H₂O+THF]. Pure fractions were combined and lyophilized to afford tert-butyl (R)-3-(2-fluoro-N-(1-methyl-1H-pyrazolo[3,4-c] pyridin-7-yl)-4-(pyrimidin-2-ylamino)benzamido)piperidine-1-carboxylate (40 mg, 54%) as an off-white solid. LC-MS: [M+H⁺]=547.40.

### Step 3:

To a solution of tert-butyl (R)-3-(2-fluoro-N-(1-methyl-1H-pyrazolo[3,4-c] pyridin-7-yl)-4-(pyrimidin-2-ylamino)benzamido)piperidine-1-carboxylate (35 mg, 0.064 mmol) in DCM (2 mL) at 0° C. was added 4M HCl in 1,4 dioxane (2.0 mL). The reaction was warmed to room temperature and stirred for 2 h. The reaction mixture was concentrated under reduced pressure. The aq. layer was washed with diethyl ether, and lyophilized to afford the title compound hydrochloride salt (28 mg, 93%) as an off-white solid. LC-MS: [M+H⁺]=447.20. ¹H NMR (CD₃OD, 400 MHZ): δ 8.74-8.65 (m, 2H), 8.21-8.20 (m, 1H), 8.16-8.09 (m, 1H), 7.89-7.75 (m, 1H), 7.59-7.47 (m, 1H), 7.15-7.05 (m, 3H), 5.15-5.01 (m, 1H), 4.23 (s, 3H), 3.95-3.92 (m, 1H), 3.72-3.67 (m, 1H), 3.37-3.29 (m, 1H), 2.97-2.89 (m, 1H), 2.16-1.92 (m, 3H), 1.33-1.29 (m, 1H).

### Synthesis of (R)-2-fluoro-4-(1H-isoindol-3-yl)-N-(8-methylisoquinolin-1-yl)-N-(piperidin-3-yl)benzamide (Example 120)

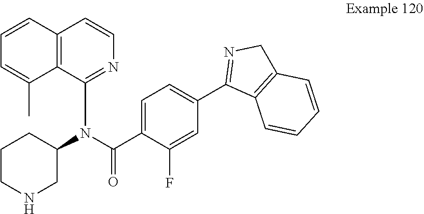 [SMILES: Cc1cccc2ccnc(N(C(=O)c3ccc(C4=NCc5ccccc54)cc3F)[C@@H]3CCCNC3)c12]

The synthesis of Example 120 was performed according to the procedure shown for Example 127 by substituting 3-iodo-4,5,6,7-tetrahydro-1H-indazole with 2-bromo-5-methyl-1,3,4-thiadiazole, provided the title compound hydrochloride salt as a white solid. LC-MS: [M+H⁺]=480.20. ¹H NMR (CD₃OD, 400 MHZ): δ 8.45-8.42 (m, 1H), 7.78-7.70 (m, 2H), 7.65-7.61 (m, 1H), 7.52-7.46 (m, 3H), 7.42-7.32 (m, 2H), 7.31-7.29 (m, 1H), 7.20-7.16 (m, 1H), 6.95-6.90 (m, 1H), 5.12-4.92 (m, 1H), 4.22-4.00 (m, 1H), 3.87-3.58 (m, 1H), 3.65-3.58 (m, 1H), 3.42-3.39 (m, 1H), 3.13-3.01 (m, 1H), 2.86 (s, 3H), 1.98-1.91 (m, 2H), 1.66-1.60 (m, 1H).

### Synthesis of 2-fluoro-N-(3-methylthieno[3,2-c]pyridin-4-yl)-N-[(3R)-3-piperidyl]-4-(triazolo[4,5-b]pyridin-3-yl)benzamide (Example 121)

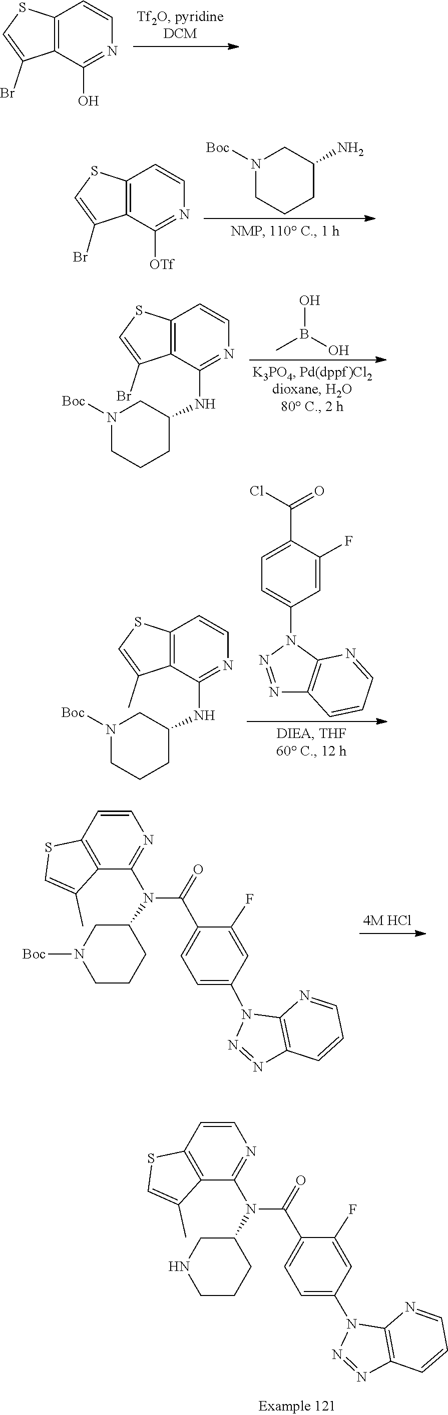

### Step 1:

To a solution of 3-bromothieno[3,2-c]pyridin-4-ol (45 g, 195.58 mmol) in DCM (1200 mL) was added pyridine (47.36 mL, 586.75 mmol,) at 20° C. The reaction mixture was cooled to 0° C. and followed by dropwise addition of Tf₂O (58.09 mL, 352.05 mmol,) under N₂ atmosphere. The mixture was stirred at 20° C. for 1 h before it was filtered. The filtrate was diluted with water (1.5 L) and then was extracted with dichloromethane (3×500 mL). The combined organic phase was washed with brine (1 L), dried over Na₂SO₄, filtered, and concentrated. The crude product was triturated with petroleum ether (200 mL) at 20° C. for 10 min, then the precipitate was collected and dried to give (3-bromothieno[3,2-c]pyridin-4-yl) trifluoromethanesulfonate (60 g, 80%) as a solid.

### Step 2:

To a solution of 3-bromothieno[3,2-c]pyridin-4-yl trifluoromethanesulfonate (50 g, 124.26 mmol, 1 eq) in NMP (500 mL) at 20° C. was added tert-butyl (3R)-3-aminopiperidine-1-carboxylate (124.43 g, 621.30 mmol). The mixture was stirred at 110° C. for 1 h before it was allowed to cool and was filtered. The filtrate was diluted with water (1500 mL) and extracted with ethyl acetate (3×1000 mL). The combined organic phase was washed with brine (3×1000 mL), dried over Na₂SO₄, filtered, and concentrated. The crude product was purified by column chromatography (SiO₂, Petroleum ether:Ethyl acetate=100:5) and concentrated under reduced pressure to give tert-butyl (3R)-3-[(3-bromothieno[3,2-c]pyridin-4-yl)amino]piperidine-1-carboxylate (20 g, 36%) as an off-white solid.

### Step 3:

To a solution of tert-butyl (3R)-3-[(3-bromothieno[3,2-c]pyridin-4-yl)amino]piperidine-1-carboxylate (20 g, 45.54 mmol) in dioxane (200 mL) and H₂O (100 mL) at 20° C. under N₂ atmosphere was added methylboronic acid (13.63 g, 227.72 mmol), K₃PO₄ (29.00 g, 136.63 mmol) and Pd(dppf)Cl₂ (3.33 g, 4.55 mmol). The mixture was then stirred at 80° C. for 1 h under N₂ atmosphere before it was cooled and poured into water (500 mL) and extracted with ethyl acetate (3×500 mL). The combined organic phase was washed with brine (00 mL), dried with anhydrous sodium sulfate, filtered, and concentrated. The crude was purified by flash column chromatography (SiO₂, Petroleum ether:Ethyl acetate=100:5) and concentrated under pressure to give tert-butyl (R)-3-((3-methylthieno[3,2-c]pyridin-4-yl)amino)piperidine-1-carboxylate (10 g, 61%) as a yellow solid.

### Step 4:

To a solution of tert-butyl (3R)-3-[(3-methylthieno[3,2-c]pyridin-4-yl)amino]piperidine-1-carboxylate (2 g, 5.76 mmol) in THF (10 mL) at 20° C. was added DIEA (5.01 mL, 28.78 mmol) and 2-fluoro-4-(triazolo[4,5-b]pyridin-3-yl)benzoyl chloride (2.87 g, 10.36 mmol). The mixture was then stirred at 60° C. for 12 h before it was concentrated under reduced pressure to give tert-butyl (3R)-3-[[2-fluoro-4-(triazolo[4,5-b]pyridin-3-yl)benzoyl]-(3-methylthieno[3,2-c]pyridin-4-yl)amino] piperidine-1-carboxylate (2.5 g, 67%) as a yellow solid.

### Step 5:

A mixture of tert-butyl (3R)-3-[2-fluoro-4-(triazolo[4,5-b]pyridin-3-yl)benzoyl]-(3-methylthieno[3,2-c]pyridin-4-yl)amino] piperidine-1-carboxylate (2.5 g, 3.91 mmol) and 4M HCl in EtOAc (25 mL) was stirred at 20° C. for 1 h. The mixture was concentrated under reduced pressure and lyophilized to give (hydrochloride salt (1.64 g, 76%) as an off-white solid. LC-MS: [M+H⁺]=488.1. ¹H NMR (400 MHZ, DMSO-d₆): δ 9.42-9.27 (m, 1H), 9.24-9.02 (m, 1H), 8.87-8.78 (m, 1H), 8.74-8.65 (m, 1H), 8.51-8.38 (m, 1H), 8.10-7.98 (m, 2H), 7.91-7.83 (m, 1H), 7.73 (s, 2H), 7.36-7.24 (m, 1H), 5.06-4.66 (m, 1H), 3.84-3.74 (m, 1H), 3.48 (br d, J=10.8 Hz, 1H), 3.30-3.17 (m, 1H), 2.81-2.68 (m, 1H), 2.62-2.55 (m, 3H), 1.87-1.68 (m, 3H), 1.15-0.98 (m, 1H).

### Synthesis of (R)-4-(3H-[1,2,3]triazolo[4,5-b]pyridin-3-yl)-N-(3-bromothieno[3,2-c]pyridin-4-yl)-2-fluoro-N-(piperidin-3-yl)benzamide (Example 122)

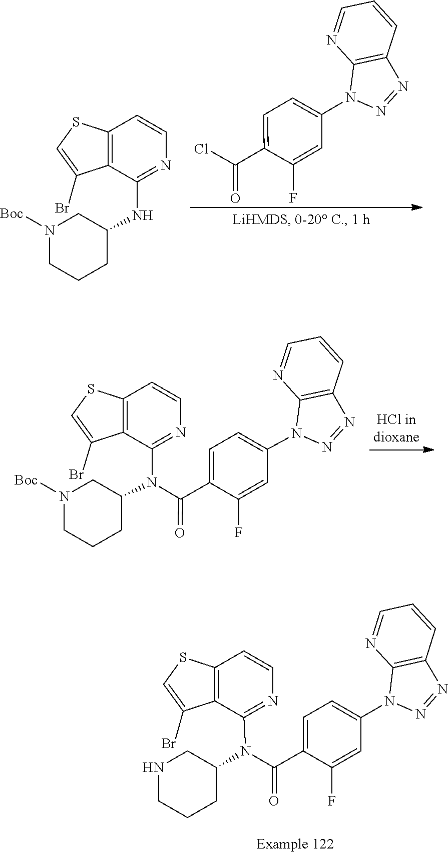

To a solution of tert-butyl (R)-3-((3-bromothieno[3,2-c]pyridin-4-yl)amino)piperidine-1-carboxylate (0.1 g, 242.52 μmol) in THF (3 mL) at 0° C. under N₂ atmosphere was added LiHMDS in THF (1 M, 364 μL). The reaction mixture was stirred at 0° C. for 0.5 h and followed by addition of 4-(3H-[1,2,3]triazolo[4,5-b]pyridin-3-yl)-2-fluorobenzoyl chloride (201.28 mg, 727.55 μmol) in THF (2 mL). The reaction mixture was stirred at 20° C. for 0.5 h before it was very slowly poured into saturated aq. NH₄Cl (10 mL) over 10 min. The aqueous phase was extracted with ethyl acetate (3×8 mL). The combined organic phase was washed with brine (8 mL), dried over anhydrous Na₂SO₄, concentrated. The crude residue was purified by prep-HPLC (column: Phenomenex Luna C18 75×30 mm×3 μm; mobile phase: [A: H₂O (0.04% HCl); B: MeCN]; B %: 50.00%-75.00%, 8.00 min) to give tert-butyl (R)-3-(4-(3H-[1,2,3]triazolo[4,5-b]pyridin-3-yl)-N-(3-bromothieno[3,2-c]pyridin-4-yl)-2-fluorobenzamido)piperidine-1-carboxylate (30 mg, 18%) as a yellow solid. LC-MS: [M+H⁺]=652.0.

### Step 2:

Example 122 was obtained as an off-white solid by deprotection Method A used in Example 97. LC-MS: [M+H⁺]=552.1. ¹H NMR (400 MHZ, DMSO-d₆): δ 9.32 (br d, J=9.26 Hz, 1H), 9.02-9.15 (m, 1H), 8.83 (d, J=4.63 Hz, 1H), 8.69 (dd, J=8.38, 1.13 Hz, 1H), 8.46-8.54 (m, 1H), 8.18-8.24 (m, 2H), 8.01 (dd, J=10.76, 1.88 Hz, 1H), 7.90 (dd, J=8.57, 1.69 Hz, 1H), 7.61 (dd, J=8.32, 4.57 Hz, 1H), 7.37-7.47 (m, 1H), 4.79-5.07 (m, 1H), 3.74-3.83 (m, 1H), 3.22 (br d, J=12.51 Hz, 1H), 2.66-2.81 (m, 1H), 1.76-1.97 (m, 3H), 0.91-1.07 (m, 1H).

### Synthesis of (R)-2-fluoro-N-(8-methylisoquinolin-1-yl)-N-(piperidin-3-yl)-4-(4,5,6,7-tetrahydro-1H-indazol-3-yl) benzamide (Example 123)

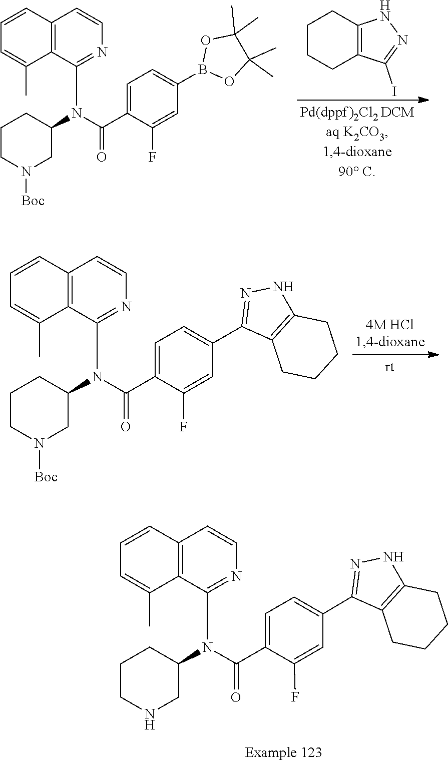

Example 123 was prepared according to the procedure as described for Example 127 by substituting 2-bromo-5-methyl-1,3,4-thiadiazole with 3-iodo-4,5,6,7-tetrahydro-1H-indazole, provided the title compound hydrochloride salt as an off-white solid. LC-MS: [M+H⁺]=484.30. ¹H NMR (CD₃OD, 400 MHZ): δ 8.48-8.45 (m, 1H), 7.79-7.75 (m, 1H), 7.71-7.61 (m, 1H), 7.58-7.53 (m, 2H), 7.21-7.15 (m, 1H), 7.08-7.02 (m, 2H), 5.14-5.11 (m, 1H), 4.07-4.04 (m, 1H), 3.89-3.83 (m, 1H), 3.47-3.44 (m, 1H), 3.30-3.27 (m, 1H), 3.08-3.01 (m, 1H), 2.90 (s, 3H), 2.80-2.74 (m, 2H), 2.58-2.55 (m, 2H), 2.00-1.84 (m, 5H), 1.63-1.3 (m, 2H).

### Synthesis of (R)-4-amino-2-fluoro-N-(8-methylisoquinolin-1-yl)-N-(piperidin-3-yl) benzamide (Example 124)

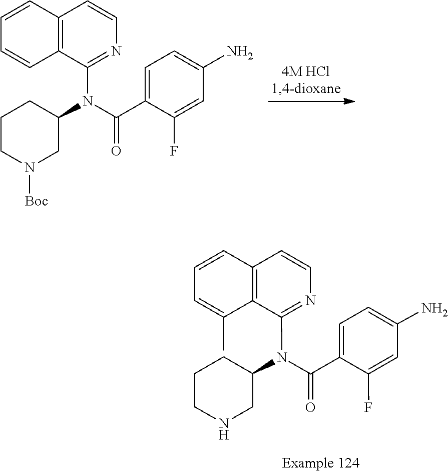 [SMILES: CC(C)(C)OC(=O)N1CCC[C@@H](N(C(=O)c2ccc(N)cc2F)c2nccc3ccccc23)C1 ; Cc1cccc2ccnc(N(C(=O)c3ccc(N)cc3F)[C@@H]3CCCNC3)c12]

To a solution of tert-butyl (R)-3-(4-amino-2-fluoro-N-(8-methylisoquinolin-1-yl) benzamido) piperidine-1-carboxylate (50 mg, 0.10 mmol) in DCM (1 mL) at 0° C. was added 4M HCl in 1,4-dioxane (0.5 mL). The reaction was warmed to room temperature and stirred for 2 h before it was concentrated under reduced pressure. The residue was further purified by Prep-HPLC (Conditions: Column: X-SELECT CSH, Mobile phase A: 0.1% formic acid in water (100%), Mobile phase B: 100% MeCN, Gradient: T/% B: 0/25, 10/50, Flow: 15 mL/min, Dilution: MeCN+H₂O+THF). Pure fractions were combined and dried by lyophilization to afford the title compound formic acid salt (25 mg, 49%) as an off-white solid. LC-MS: [M+H⁺]=379.2 ¹H NMR (CD₃OD, 400 MHZ): δ 8.53 (bs, 1H), 8.39 (d, J=5.2 Hz, 1H), 7.72 (d, J=5.2 Hz, 1H), 7.68-7.66 (m, 1H), 7.51-7.47 (m, 1H), 7.40-7.38 (m, 1H), 6.37-6.33 (m, 1H), 5.98-5.84 (m, 1H), 5.80-5.78 (m, 1H), 5.01-5.14 (m, 1H), 3.84-3.81 (m, 1H), 3.72-3.66 (m, 1H), 2.90-2.76 (m, 2H), 2.74 (s, 3H), 1.88-1.77 (m, 3H), 1.53-1.50 (m, 1H).

### Synthesis of (R)-2-fluoro-4-(2-methyl-2H-tetrazol-5-yl)-N-(8-methylisoquinolin-1-yl)-N-(piperidin-3-yl) benzamide (Example 125))

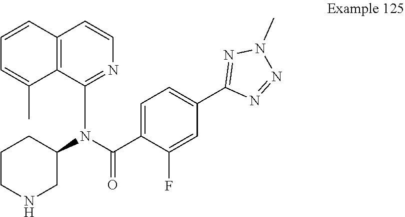 [SMILES: Cc1cccc2ccnc(N(C(=O)c3ccc(-c4nnn(C)n4)cc3F)[C@@H]3CCCNC3)c12]

Example 125 was prepared according to the procedure as described for Example 127 by substituting 2-bromo-5-methyl-1,3,4-thiadiazole with 5-bromo-2-methyl-2H-tetrazole, provided the title compound hydrochloride salt (37 mg, 92%) as an off-white solid. LC-MS: [M+H⁺]=446.20. ¹H NMR (CD₃OD, 400 MHZ): δ 8.43-8.40 (m, 1H), 7.73-7.69 (m, 1H), 7.64-7.59 (m, 1H), 7.52-7.43 (m, 3H), 7.40-7.38 (m, 1H), 6.98-6.93 (m, 1H), 5.10-5.04 (m, 1H), 4.34 (s, 3H), 4.00-3.83 (m, 1H), 3.65-3.39 (m, 1H), 3.25-3.00 (m, 1H), 2.86 (s, 3H), 1.97-1.92 (m, 2H), 1.62-1.58 (m, 1H), 1.36-1.28 (m, 1H), 0.96-0.89 (m, 1H).

### Synthesis of (R)-2-fluoro-4-(1-methyl-5-phenyl-1H-pyrazol-4-yl)-N-(8-methylisoquinolin-1-yl)-N-(piperidin-3-yl) benzamide (Example 126))

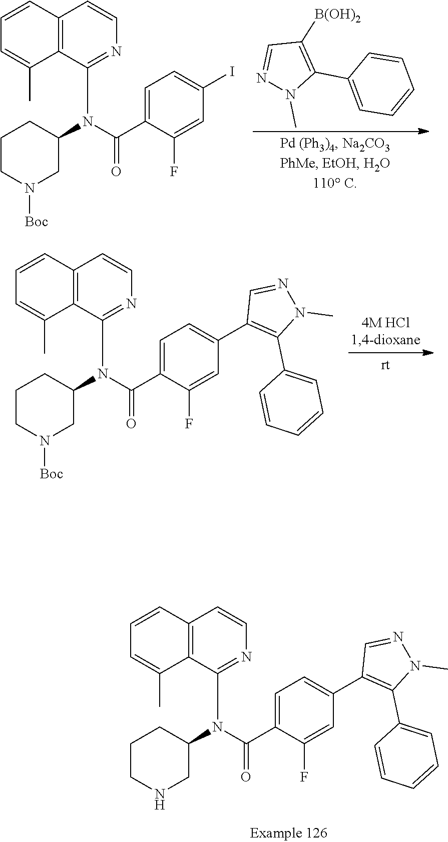

### Step 1:

To a solution of tert-butyl (R)-3-(2-fluoro-4-iodo-N-(8-methylisoquinolin-1-yl)benzamido)piperidine-1-carboxylate (100 mg, 0.17 mmol) and (1-methyl-5-phenyl-1H-pyrazol-4-yl)boronic acid (68 mg, 0.34 mmol) in a mixture of DME and H₂O (1:1/v:v, 2 mL) at room temperature was added Na₂CO₃ (53 mg, 0.51 mmol). The reaction mixture was purged with nitrogen for 10 min and followed by addition of Pd(dppf)Cl₂·CH₂Cl₂ (7 mg, 0.008 mmol). The reaction was then heated at 110° C. for 16 h before it was cooled. The reaction mixture was filtered through celite, and the filtrate was concentrated. The residue was diluted with water (30 mL) and extracted with EtOAc (2×50 mL). The combined organic layer was dried over Na₂SO₄ and concentrated. The crude product was purified by Combi-flash using 80% EtOAc in hexane first and then further purified by prep-HPLC (Conditions: LUNA C-18 (250×21.2×5 μm), Mobile phase A: 0.1% formic acid in water (100%), Mobile phase B: 100% MeCN, Gradient: Time (min)/% B: 0/70, 10/90, Flow: 15 mL/min, Dilution: MeCN+H₂O+THF). Pure fractions were combined and dried by lyophilization to afford tert-butyl (R)-3-(2-fluoro-4-(1-methyl-5-phenyl-1H-pyrazol-4-yl)-N-(8-methylisoquinolin-1-yl) benzamido)piperidine-1-carboxylate formate salt (60 mg, 75%) as an off white solid. LC-MS: [M+H⁺]=620.30.

### Step 2:

The title compound hydrochloride salt was obtained as an off-white solid by deprotection Method A used in Example 97. LC-MS: [M+H⁺]=520.25. ¹H NMR (CD₃OD, 400 MHZ): δ 8.36-8.32 (m, 1H), 7.78 (s, 1H), 7.74-7.68 (m, 2H), 7.61-7.59 (m, 4H), 7.42-7.39 (m, 1H), 7.10-7.08 (m, 2H), 6.61-6.57 (m, 1H), 6.51-6.47 (m, 1H), 6.44-6.41 (m, 1H), 5.05-4.93 (m, 1H), 3.93-3.90 (m, 1H), 3.77-3.71 (m, 1H), 3.68 (s, 3H), 3.38-3.32 (m, 1H), 2.99-2.95 (m, 1H), 2.74 (s, 3H), 2.15-1.91 (m, 2H), 1.59-1.55 (m, 1H), 1.35-1.26 (m, 1H).

### Synthesis of (R)-2-fluoro-4-(5-methyl-1,3,4-thiadiazol-2-yl)-N-(8-methylisoquinolin-1-yl)-N-(piperidin-3-yl) benzamide (Example 127)

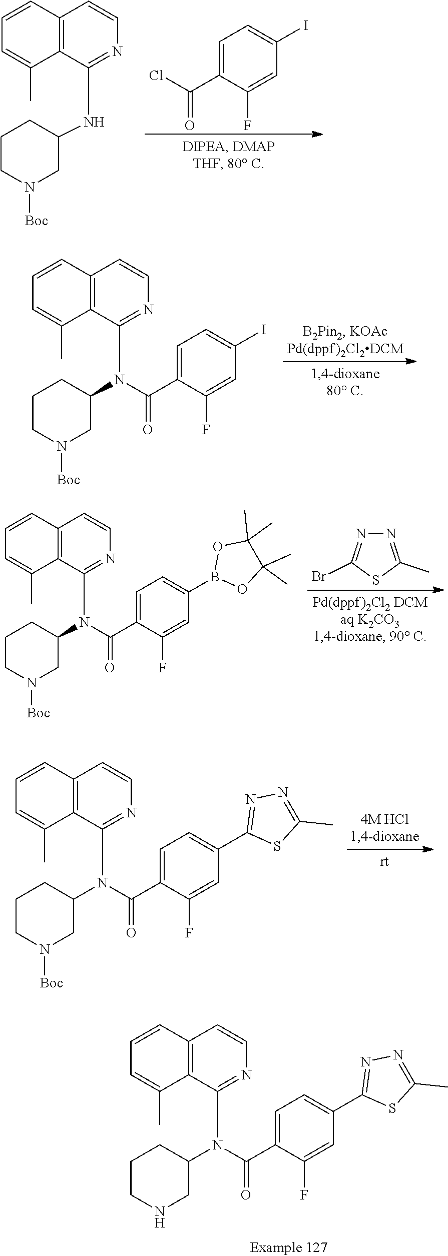

### Step 1:

To a stirred solution of tert-butyl (R)-3-((8-methylisoquinolin-1-yl)amino)piperidine-1-carboxylate (1.5 g, 4.39 mmol) in THF (15 mL) at room temperature was added 2-fluoro-4-iodobenzoyl chloride (3.7 g, 13.18 mmol), 4-DMAP (107 mg, 0.878 mmol) and DIPEA (4.8 mL, 26.34 mmol). The reaction mixture was stirred at 80° C. for 16 h before it was cooled. The reaction mixture was diluted with water (10 mL) and extracted with ethyl acetate (2×20 mL). The combined organic layer was washed with brine (10 mL), dried over Na₂SO₄, and concentrated under reduced pressure. The crude product was purified by combi-flash using 24% EtOAc in hexane to afford tert-butyl (R)-3-(2-fluoro-4-iodo-N-(8-methylisoquinolin-1-yl)benzamido)piperidine-1-carboxylate (2.3 g, 88%) as an off-white solid. LC-MS: [M+H⁺]=590.15.

### Step 2:

To a stirred solution of tert-butyl (R)-3-(2-fluoro-4-iodo-N-(8-methylisoquinolin-1-yl)benzamido)piperidine-1-carboxylate (1 g, 1.69 mmol) in 1,4-dioxane (10 mL) at 25° C. was added bis(pinacolato)diboron (1.3 g, 5.08 mmol), KOAc (490 mg, 5.08 mmol) and PdCl₂(dppf)·DCM (69 mg, 0.0845 mmol). The reaction mixture was degassed with N₂ for 5 min and then stirred at 90° C. for 16 h. The reaction mixture was cooled, diluted with water (10 mL), and extracted with EtOAc (2×20 mL). The combined organic layer was washed with brine (10 mL), dried over Na₂SO₄, and concentrated. The crude product was purified by Combi-flash using 32% EtOAc in hexane to afford tert-butyl (R)-3-(2-fluoro-N-(8-methylisoquinolin-1-yl)-4-(4,4,5,5-tetramethyl-1,3,2-dioxaborolan-2-yl)benzamido)-piperidine-1-carboxylate (800 mg) as an off-white solid. LC-MS: [M+H⁺]=590.3.

### Step 3:

To a solution of 2-bromo-5-methyl-1,3,4-thiadiazole (80 mg, 0.44 mmol) and tert-butyl(R)-3-(2-fluoro-N-(8-methylisoquinolin-1-yl)-4-(4,4,5,5-tetramethyl-1,3,2dioxaborolan-2-yl)benzamido)piperidine-1-carboxylate (200 mg, 0.44 mmol) in a mixture of DME (1 mL) and H₂O (1 mL) at 25° C. was added Na₂CO₃ (138 mg, 1.3 mmol). The reaction mixture was degassed with nitrogen for 10 min and followed by addition of Pd(dppf)Cl₂·CH₂Cl₂ (18 mg, 0.02 mmol). The mixture was heated to 100° C. for 16 h before it was diluted with water (30 mL) and extracted with EtOAc (2×10 mL). The combined organic layer was washed with brine, dried over Na₂SO₄, filtered, and concentrated. The crude product was purified by Combi-flash using 60% EtOAc in hexane to afford tert-butyl (R)-3-(2-fluoro-4-(5-methyl-1,3,4-thiadiazol-2-yl)-N-(8-methylisoquinolin-1-yl) benzamido)piperidine-1-carboxylate (40 mg) as a yellow solid. This solid was further purified by prep-HPLC (Conditions: LUNA C-18 (250×21.2×5 μm), Mobile phase A: 0.1% formic acid in water, Mobile phase B: 100% MeCN, Gradient: Tim (min)/% B: 0/70, 10/90, Flow: 15 mL/min, Dilution: MeCN+H₂O+THF). The pure fractions were combined and dried by lyophilization to afford tert-butyl (R)-3-(2-fluoro-4-(5-methyl-1,3,4-thiadiazol-2-yl)-N-(8-methylisoquinolin-1-yl)benzamido)piperidine-1-carboxylate formate salt (30 mg, 69%) as an off white solid. LC-MS: [M+H⁺]=562.20.

### Step 4:

To a solution of tert-butyl (R)-3-(2-fluoro-4-(5-methyl-1,3,4-thiadiazol-2-yl)-N-(8-methylisoquinolin-1-yl)benzamido)piperidine-1-carboxylate formate salt (30 mg, 0.04 mmol) in DCM (1 mL) was added 4M HCl in 1,4-dioxane (0.5 mL) at 0° C. and the reaction mixture was stirred at room temperature for 2 h. The reaction mixture was concentrated under reduced pressure and dried by lyophilization to afford (R)-2-fluoro-4-(5-methyl-1,3,4-thiadiazol-2-yl)-N-(8-methylisoquinolin-1-yl)-N-(piperidin-3-yl) benzamide hydrochloride salt (29 mg, 83%) as an off-white solid. LC-MS: [M+H⁺]=462.20. ¹H NMR (CD₃OD, 400 MHz): δ 8.43 (d, J=5.2 Hz, 1H), 7.74 (d, J=4.8 Hz, 1H), 7.66 (d, J=7.2 Hz, 1H), 7.54-7.50 (m, 2H), 7.41 (d, J=10.8 Hz, 1H), 7.25 (d, J=7.6 Hz, 1H), 7.00-6.96 (m, 1H), 5.12-5.02 (m, 1H), 4.02-4.00 (m, 1H), 3.85-3.79 (m, 1H), 3.48-3.39 (m, 1H), 3.04-3.01 (m, 1H), 2.75 (s, 3H), 2.02 (s, 3H), 2.00-1.96 (m, 3H), 1.62-1.52 (m, 1H).

### Synthesis of(S)—N-(8-methylisoquinolin-1-yl)-N-(piperidin-3-yl)-4-(1H-tetrazol-5-yl)benzamide (Example 128)

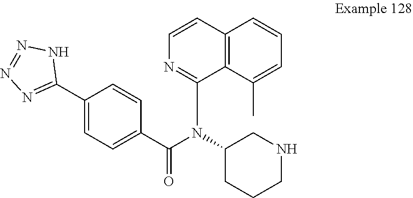 [SMILES: Cc1cccc2ccnc(N(C(=O)c3ccc(-c4nnn[nH]4)cc3)[C@H]3CCCNC3)c12]

Example 128 was prepared according to the procedure as described for Example 111 by substituting 4-(1-methyl-1H-1,2,3-triazol-4-yl)benzoic acid with 4-cyanobenzoic acid and followed by converting the corresponding nitrile to the tetrazole, provided the title compound hydrochloride salt as an off-white solid. LC-MS: [M+H⁺]=414.3. ¹H NMR (400 MHz, CD3OD): δ 8.54-8.46 (m, 1H), 7.84-7.77 (m, 1H), 7.70-7.59 (m, 3H), 7.47 (t, J=7.6 Hz, 1H), 7.43-7.36 (m, 1H), 7.25-7.18 (m, 2H), 5.16-5.06 (m, 1H), 4.02-3.94 (m, 1H), 3.88-3.79 (m, 1H), 3.40 (br d, J=12.5 Hz, 1H), 3.06-2.95 (m, 1H), 2.77 (s, 3H), 2.01-1.91 (m, 2H), 1.87 (br d, J=13.0 Hz, 1H), 1.60-1.46 (m, 1H).

### Synthesis of methyl (R)-1-(4-(3H-[1,2,3]triazolo[4,5-b]pyridin-3-yl)-2-fluoro-N-(piperidin-3-yl)benzamido)isoquinoline-7-carboxylate (Example 129)

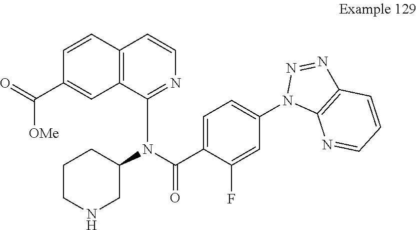 [SMILES: COC(=O)c1ccc2ccnc(N(C(=O)c3ccc(-n4nnc5cccnc54)cc3F)[C@@H]3CCCNC3)c2c1]

Example 129 was prepared according to the procedure described for Example 117 by substituting 6-bromo-1-chloroisoquinoline with 7-bromo-1-chloroisoquinoline, provided the title compound as an off-white solid. LC-MS: [M+H⁺]=526.20. ¹H NMR (CD₃OD, 400 MHz): δ 8.85-8.74 (m, 1H), 8.64-8.51 (m, 2H), 8.23-8.18 (m, 1H), 8.09-7.70 (m, 5H), 7.55-7.53 (m, 1H), 7.35-7.30 (m, 1H), 5.37-5.21 (m, 1H), 4.07-4.04 (bs, 3H), 3.76-3.74 (m, 1H), 2.95-2.88 (m, 1H), 2.72-2.61 (m, 1H), 2.16-1.93 (m, 3H), 1.37-1.30 (m, 2H).

### Synthesis of ethyl (R,E)-3-(1-(4-(3H-[1,2,3]triazolo[4,5-b]pyridin-3-yl)-2-fluoro-N-(piperidin-3-yl)benzamido)isoquinolin-7-yl)acrylate (Example 130)

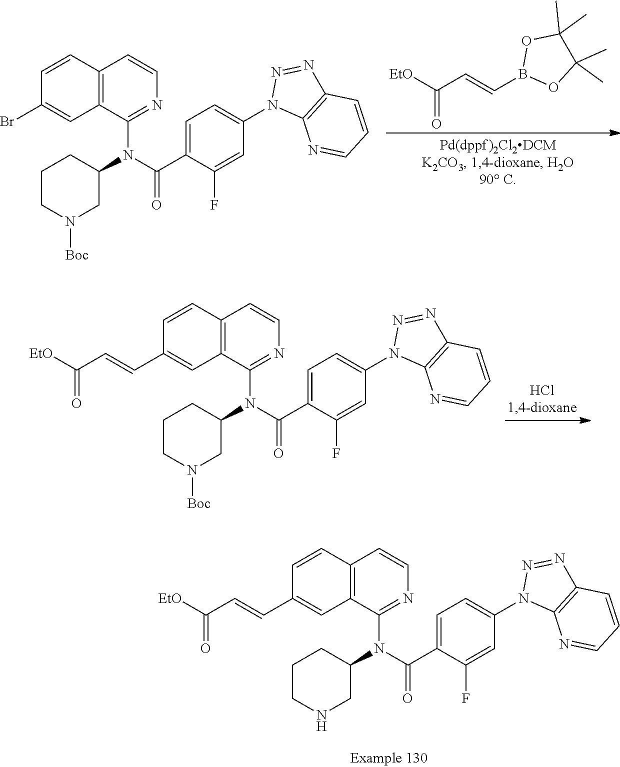 [SMILES: CC(C)(C)OC(=O)N1CCC[C@@H](N(C(=O)c2ccc(-n3nnc4cccnc43)cc2F)c2nccc3ccc(Br)cc23)C1 ; CCOC(=O)/C=C/B1OC(C)(C)C(C)(C)O1 ; CCOC(=O)/C=C/c1ccc2ccnc(N(C(=O)c3ccc(-n4nnc5cccnc54)cc3F)[C@@H]3CCCN(C(=O)OC(C)(C)C)C3)c2c1 ; CCOC(=O)/C=C/c1ccc2ccnc(N(C(=O)c3ccc(-n4nnc5cccnc54)cc3F)[C@@H]3CCCNC3)c2c1]

### Step 1:

To a solution of tert-butyl (R)-3-(4-(3H-[1,2,3]triazolo[4,5-b]pyridin-3-yl)-N-(7-bromoisoquinolin-1-yl)-2-fluorobenzamido)piperidine-1-carboxylate (300 mg, 0.463 mmol) in 1,4-dioxane (5 mL) and water (1 mL) at room temperature were added ethyl (E)-3-(4,4,5,5-tetramethyl-1,3,2-dioxaborolan-2-yl)acrylate (125 mg, 0.552 mmol) and K₂CO₃ (190 mg, 1.376 mmol). The reaction mixture was purged with nitrogen gas for 5 min and followed by addition of Pd(dppf)₂Cl₂·DCM (16 mg, 0.021 mmol). The reaction mixture was heated to 90° C. and stirred for 16 h and then it was diluted with water (20 mL) and extracted with EtOAc (2×20 mL). The combined organic layer was washed with brine, dried over Na₂SO₄, and concentrated under reduced pressure. The crude residue was purified by Combi-flash using 60-70% EtOAc in hexane to give a solid which was further purified by prep-HPLC (Conditions: Luna OMEGA C-18 (250×21.2×5 μm), Mobile phase A: 0.1% formic acid in water (100%), Mobile phase B: 100% MeCN, Gradient: T/% B: 1/10, 2/10, 10/50, Flow: 15 mL/min, Dilution: MeCN+H₂O+THF). Pure fractions were combined and dried by lyophilization to afford tert-butyl (R,E)-3-(4-(3H-[1,2,3]triazolo[4,5-b]pyridin-3-yl)-N-(7-(3-ethoxy-3-oxoprop-1-en-1-yl)isoquinolin-1-yl)-2-fluorobenzamido)piperidine-1-carboxylate (45 mg, 76%) as an off-white solid. LC-MS: [M+H⁺]=666.20.

### Step 2:

The title compound hydrochloride salt was obtained as an off-white solid by deprotection Method A used in Example 97. LC-MS: [M+H⁺]=566.3. ¹H NMR (CD₃OD, 400 MHZ): δ 9.52-9.68 (m, 1H), 9.33-9.29 (m, 2H), 9.12-9.06 (m, 1H), 8.85-8.79 (m, 2H), 8.74-8.67 (m, 3H), 8.58-8.54 (m, 1H), 8.35-8.31 (m, 1H), 8.11-8.08 (m, 1H), 7.59-7.55 (m, 1H), 6.15-6.12 (m, 1H), 5.13-5.02 (m, 2H), 4.80-4.77 (m, 1H), 4.57-4.47 (m, 1H), 3.72-3.68 (m, 1H), 2.85-2.72 (m, 3H), 2.20-2.09 (m, 4H), 1.76-1.69 (m, 1H).

### Synthesis of (R)-2-fluoro-4-((1-methyl-2-oxo-1,2-dihydropyridin-3-yl)amino)-N-(8-methylisoquinolin-1-yl)-N-(piperidin-3-yl)benzamide (Example 131)

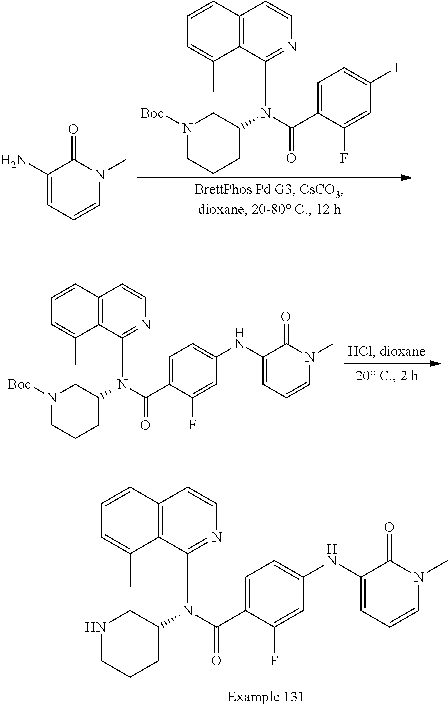

### Step 1:

To a solution of tert-butyl (R)-3-(2-fluoro-4-iodo-N-(8-methylisoquinolin-1-yl)benzamido)piperidine-1-carboxylate (0.1 g, 169.65 μmol) in dioxane (2 mL) at 20° C. under N₂ atmosphere was added 3-amino-1-methyl-pyridin-2-one (25.27 mg, 203.58 μmol), Cs₂CO₃ (165.83 mg, 508.96 μmol) and BrettPhos Pd G3 (15.38 mg, 16.97 μmol). The reaction mixture was stirred at 80° C. for 12 h before it was allowed to cool. The reaction mixture was filtered and concentrated under reduced pressure. The residue was purified by prep-HPLC (column: Phenomenex Luna C18 75*30 mm*3 μm; mobile phase: [A: H₂O (0.04% HCl); B: MeCN]; B %: 55.00%-85.00%, 8.00 min) to tert-butyl (R)-3-(2-fluoro-4-((1-methyl-2-oxo-1,2-dihydropyridin-3-yl)amino)-N-(8-methylisoquinolin-1-yl)benzamido)piperidine-1-carboxylate (50 mg, 48%) as a yellow solid. LC-MS: [M+H⁺]=586.5.

### Step 2:

The title compound hydrochloride salt was obtained as an off-white solid by deprotection Method A used in Example 97. LC-MS: [M+H⁺]=486.3. ¹H NMR (400 MHZ, DMSO-d₆): δ 9.21-9.40 (m, 1H), 9.00-9.14 (m, 1H), 8.39-8.49 (m, 1H), 7.70-7.99 (m, 3H), 7.41-7.55 (m, 2H), 7.23 (dd, J=6.82, 1.44 Hz, 1H), 6.94 (dd, J=7.38, 1.50 Hz, 1H), 6.43-6.67 (m, 3H), 6.12 (t, J=7.07 Hz, 1H), 4.73-4.99 (m, 1H), 3.72 (br d, J=10.88 Hz, 1H), 3.48-3.59 (m, 1H), 3.44 (s, 3H), 3.19-3.27 (m, 1H), 2.69-2.89 (m, 4H), 1.64-1.91 (m, 3H), 1.20-1.37 (m, 1H).

### Synthesis of (R)-2-fluoro-N-(8-methylisoquinolin-1-yl)-4-((2-oxo-1,2-dihydropyridin-3-yl)amino)-N-(piperidin-3-yl)benzamide (Example 132)

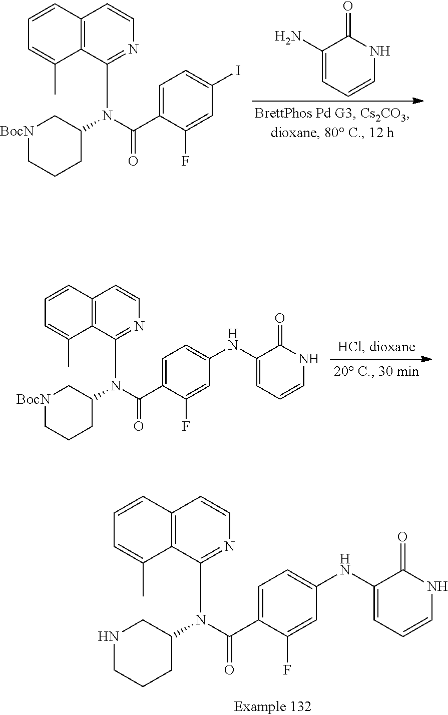

Example 132 was prepared according to the procedure described for Example 131 by substituting 3-amino-1-methyl-pyridin-2-one with 3-aminopyridin-2 (1H)-one, provided the title compound hydrochloride salt as an off-white solid. LC-MS: [M+H⁺]=472.30. ¹H NMR (400 MHZ, CD₃OD): δ 8.44 (d, J=5.5 Hz, 1H), 7.79 (d, J=5.5 Hz, 1H), 7.71 (d, J=8.0 Hz, 1H), 7.54 (t, J=7.6 Hz, 1H), 7.45 (br d, J=7.1 Hz, 1H), 7.14 (dd, J=1.5, 7.4 Hz, 1H), 7.03 (dd, J=1.6, 6.6 Hz, 1H), 6.66 (t, J=8.3 Hz, 1H), 6.53 (dd, J=2.1, 12.9 Hz, 1H), 6.43-6.27 (m, 2H), 5.12-4.99 (m, 1H), 4.04-3.90 (m, 1H), 3.89-3.75 (m, 1H), 3.40 (br d, J=12.5 Hz, 1H), 3.00 (dt, J=4.9, 11.9 Hz, 1H), 2.79 (s, 3H), 2.03-1.82 (m, 3H), 1.69-1.50 (m, 1H).

### Synthesis of 2-fluoro-N-(8-methyl-1-isoquinolyl)-N-(3-piperidyl)-4-pyrimidin-2-yloxy-benzamide (Example 133)

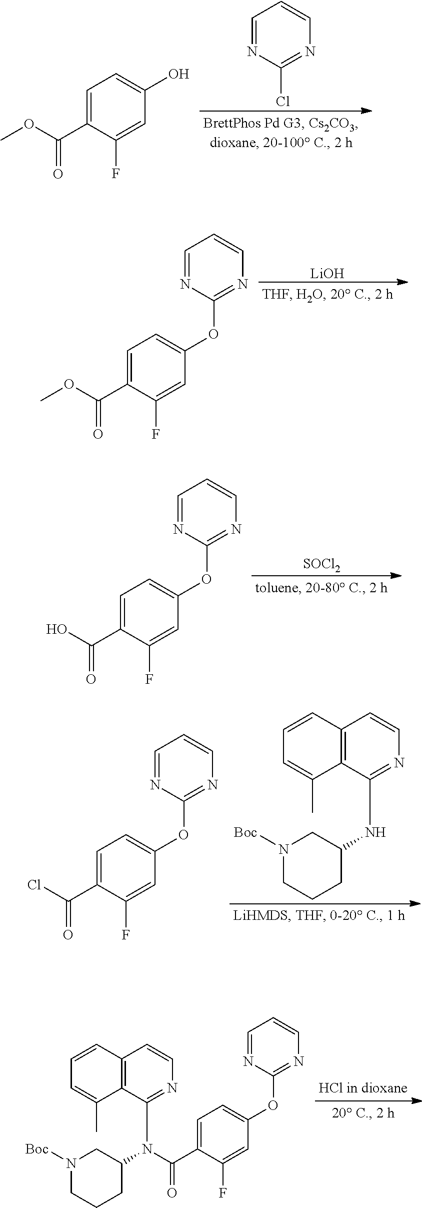

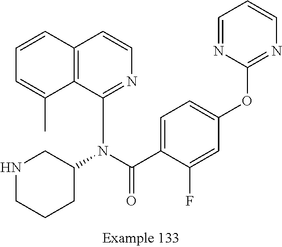

### Step 1:

To a solution of methyl 2-fluoro-4-hydroxy-benzoate (500 mg, 2.94 mmol) in dioxane (10 mL) at 20° C. under N₂ atmosphere was added 2-chloropyrimidine (403.91 mg, 3.53 mmol), Cs₂CO₃ (1.92 g, 5.88 mmol) and (1E,4E)-1,5-diphenylpenta-1,4-dien-3-one palladium (269.11 mg, 293.88 μmol). The reaction mixture was stirred at 100° C. for 2 h before it was allowed to cool. The reaction mixture was filtered and concentrated under reduced pressure. The residue was purified by flash column chromatography (SiO₂, Petroleum ether/ethyl acetate=3/1 to 1/1) to give methyl 2-fluoro-4-pyrimidin-2-yloxy-benzoate (350 mg, 45%) as a white solid.

### Step 2:

To a solution of methyl 2-fluoro-4-pyrimidin-2-yloxy-benzoate (300 mg, 1.21 mmol) in THF (4 mL) and H₂O (1 mL) was added LiOH·H₂O (152.16 mg, 3.63 mmol) at 20° C. The reaction mixture was stirred at 20° C. for 2 h before it was adjusted to pH 2 with 1 M aq. HCl solution. The precipitate was collected by filtration and dried to give 2-fluoro-4-pyrimidin-2-yloxy-benzoic acid (200 mg, 63%) as a white solid.

### Step 3:

To a solution of give 2-fluoro-4-pyrimidin-2-yloxy-benzoic acid (150 mg, 640.52 μmol) in toluene (0.5 mL) was added SOCl₂ (2 mL) at 20° C. under N₂. The mixture was heated to 80° C. and stirred for 2 h under N₂. The reaction mixture was concentrated under reduced pressure to give crude 2-fluoro-4-pyrimidin-2-yloxy-benzoyl chloride (150 mg, 74% yield) as a yellow solid.

### Step 4:

To a solution of tert-butyl 3-[(8-methyl-1-isoquinolyl)amino]piperidine-1-carboxylate (0.13 g, 380.73 μmol) in THF (6 mL) at 0° C. under N₂ atmosphere was added LiHMDS in THF (1 M, 1.14 mL). The mixture was stirred at 0° C. for 0.5 h and followed by addition of 2-fluoro-4-pyrimidin-2-yloxy-benzoyl chloride (144.28 mg, 571.10 μmol) in THF (3 mL). The reaction was stirred at 20° C. for 0.5 h before it was poured into saturated aq. NH₄Cl (8 mL) very slowly over 10 min. The aqueous phase was extracted with ethyl acetate (3×5 mL). The combined organic phase was washed with brine (5 mL), dried over anhydrous Na₂SO₄, concentrated. The residue was purified by prep-HPLC (column: Phenomenex Luna C18 75*30 mm*3 μm; mobile phase: [H₂O (0.04% HCl)-MeCN]; gradient: 50%-80% B over 8.0 min) to give tert-butyl 3-[(2-fluoro-4-pyrimidin-2-yloxy-benzoyl)-(8-methyl-1-isoquinolyl)amino]piperidine-1-carboxylate (80 mg, 35%) as an yellow solid.

### Step 5:

The title compound hydrochloride salt was obtained as an off-white solid by deprotection Method A used in Example 97. LC-MS: [M+H⁺]=458.4. ¹H NMR (400 MHZ, DMSO-d₆): δ 9.42-9.60 (m, 1H), 9.14-9.29 (m, 1H), 8.58 (d, J=4.77 Hz, 2H), 8.40-8.48 (m, 1H), 7.77-7.85 (m, 2H), 7.59 (t, J=7.65 Hz, 1H), 7.48-7.52 (m, 1H), 7.26 (t, J=4.77 Hz, 1H), 6.79-6.95 (m, 2H), 6.60 (dd, J=8.53, 1.88 Hz, 1H), 4.76-5.01 (m, 1H), 3.77 (br d, J=11.42 Hz, 1H), 3.45-3.64 (m, 1H), 3.22-3.29 (m, 1H), 2.76-2.86 (m, 4H), 1.75-1.99 (m, 3H), 1.20-1.39 (m, 1H).

### Synthesis of (R)-4-(3H-[1,2,3]triazolo[4,5-b]pyridin-3-yl)-2-fluoro-N-(7-(3 hydroxypropyl)isoquinolin-1-yl)-N-(piperidin-3-yl)benzamide (Example 134)

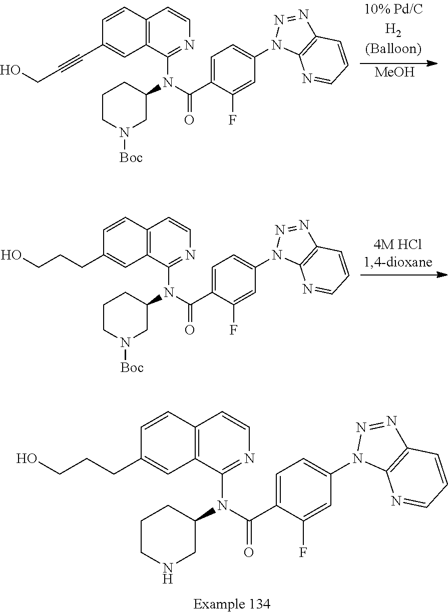 [SMILES: "MeOH" ; CC(C)(C)OC(=O)N1CCC[C@@H](N(C(=O)c2ccc(-n3nnc4cccnc43)cc2F)c2nccc3ccc(C#CCO)cc23)C1 ; CC(C)(C)OC(=O)N1CCC[C@@H](N(C(=O)c2ccc(-n3nnc4cccnc43)cc2F)c2nccc3ccc(CCCO)cc23)C1 ; O=C(c1ccc(-n2nnc3cccnc32)cc1F)N(c1nccc2ccc(CCCO)cc12)[C@@H]1CCCNC1]

### Step 1:

To a solution of tert-butyl (R)-3-(4-(3H-[1,2,3]triazolo[4,5-b]pyridin-3-yl)-2-fluoro-N-(7-(3-hydroxyprop-1-yn-1-yl)isoquinolin-1-yl)benzamido)piperidine-1-carboxylate (90 mg, 0.144 mmol) in MeOH (5 mL) was added 10% Pd/C (10 mg). The reaction mixture was stirred under a H₂ balloon at room temperature for 1 h before it was diluted with methanol and filtrated through celite. The filtrate was concentrated under reduced pressure. The residue was purified by prep-HPLC (Conditions: X-SELECT CSH, Mobile phase A: 0.1% formic acid in water (100%), Mobile phase B: 100% MeCN, Gradient: T/% B: 0/80, 10/95, Flow: 15 mL/min, Dilution: MeCN+H₂O+THF). Pure fractions were combined and dried by lyophilization to afford tert-butyl (R)-3-(4-(3H-[1,2,3]triazolo[4,5-b]pyridin-3-yl)-2-fluoro-N-(7-(3-hydroxypropyl)isoquinolin-1-yl)benzamido)piperidine-1-carboxylate formate salt (20 mg, 40%) as an off white solid. LC-MS: [M+H⁺]=626.30.

### Step 2:

The title compound hydrochloride salt was obtained as a white solid by deprotection Method A used in Example 97. LC-MS: [M+H⁺]=526.20. ¹H NMR (CD₃OD, 400 MHZ): δ 8.75-8.74 (m, 1H), 8.49 (d, J=8.4 Hz, 1H), 8.43-8.38 (m, 1H), 8.04 (dd, J=2 Hz, J=11.2 Hz, 1H), 7.93-7.87 (m, 2H), 7.78-7.73 (m, 2H), 7.66-7.59 (m, 1H), 7.54-7.50 (m, 1H), 7.30-7.26 (m, 1H), 5.34-5.26 (m, 1H), 3.97-3.94 (m, 1H), 3.70-3.60 (m, 3H), 3.22-3.12 (m, 1H), 2.97-2.87 (m, 3H), 1.98-1.89 (m, 5H), 1.31-1.20 (m, 1H).

### Synthesis of (R)-4-(3H-[1,2,3]triazolo[4,5-b]pyridin-3-yl)-2-fluoro-N-(7-(3-hydroxyprop-1-en-1-yl)isoquinolin-1-yl)-N-(piperidin-3-yl)benzamide (Example 135)

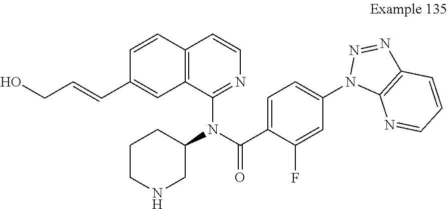 [SMILES: O=C(c1ccc(-n2nnc3cccnc32)cc1F)N(c1nccc2ccc(/C=C/CO)cc12)[C@@H]1CCCNC1]

Example 135 was prepared according to the procedure described for Example 130 by substituting ethyl (E)-3-(4,4,5,5-tetramethyl-1,3,2-dioxaborolan-2-yl)acrylate with 1-(tetrahydro-2H-pyran-2-yl)-3-(4,4,5,5-tetramethyl-1,3,2-dioxaborolan-2-yl)-1H-pyrazole, provided the title compound hydrochloride salt as a light brown solid. LC-MS: [M+H⁺]=524.25. ¹H NMR (CD₃OD, 400 MHZ): δ 8.73 (d, J=4.0 Hz, 1H), 8.54 (s, 1H), 8.48 (d, J=8.4 Hz, 1H), 8.41-8.36 (m, 1H), 8.02-7.99 (m, 2H), 7.89-7.85 (m, 2H), 7.79-7.75 (m, 1H), 7.70-7.67 (m, 1H), 7.52-7.49 (m, 1H), 7.34-7.25 (m, 1H), 6.91-6.86 (m, 1H), 6.67-6.60 (m, 1H), 5.16-5.13 (m, 1H), 4.34 (d, J=4.8 Hz, 2H), 3.81-3.79 (m, 1H), 3.50-3.44 (m, 1H), 3.17-3.06 (m, 1H), 2.76-2.56 (m, 1H), 2.19-1.76 (m, 3H), 1.28-1.26 (m, 1H).

### Synthesis of (R)—N-(3-(1H-pyrazol-3-yl)pyridin-2-yl)-4-(3H-[1,2,3]triazolo[4,5-b]pyridin-3-yl)-2-fluoro-N-(piperidin-3-yl)benzamide (Example 136)

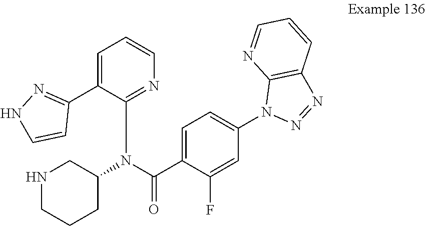 [SMILES: O=C(c1ccc(-n2nnc3cccnc32)cc1F)N(c1ncccc1-c1cc[nH]n1)[C@@H]1CCCNC1]

Example 136 was prepared according to the procedure described for Example 104 by substituting tert-butyl-dimethyl-[(E)-4-(4,4,5,5-tetramethyl-1,3,2-dioxaborolan-2-yl)but-3-enoxy]silane with 1-(tetrahydro-2H-pyran-2-yl)-3-(4,4,5,5-tetramethyl-1,3,2-dioxaborolan-2-yl)-1H-pyrazole, provided the title compound hydrochloride salt as a light brown solid. LC-MS: [M+H⁺]=484.4. ¹H NMR (400 MHZ, DMSO-d₆): δ 9.3-9.5 (m, 1H), 9.0-9.2 (m, 1H), 8.85 (dd, 1H, J=1.4, 4.5 Hz), 8.70 (dd, 1H, J=1.4, 8.4 Hz), 8.5-8.6 (m, 1H), 7.8-8.1 (m, 4H), 7.6-7.7 (m, 1H), 7.41 (dd, 1H, J=4.6, 7.9 Hz), 6.8-7.0 (m, 1H), 6.5-6.6 (m, 1H), 5.0-5.1 (m, 1H), 3.66 (br d, 1H, J=10.8 Hz), 3.3-3.5 (m, 1H), 3.22 (br d, 1H, J=11.6 Hz), 2.7-2.8 (m, 1H), 1.7-2.0 (m, 3H), 1.2-1.4 (m, 1H).

### Synthesis of (R,E)-4-(3H-[1,2,3]triazolo[4,5-b]pyridin-3-yl)-2-fluoro-N-(3-(4-hydroxybut-1-en-1-yl)-4-methylpyridin-2-yl)-N-(piperidin-3-yl)benzamide (Example 137)

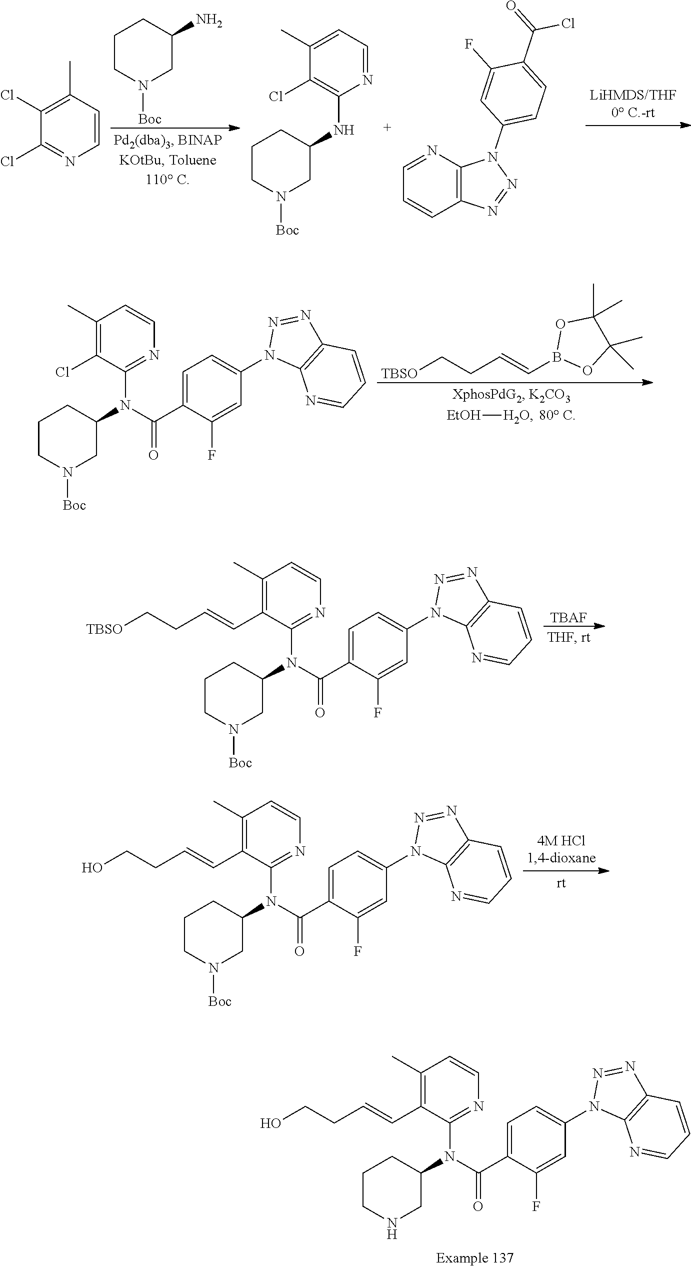

### Step 1:

To a solution of 2,3-dichloro-4-methylpyridine (3 g, 18.51 mmol) and tert-butyl (R)-3-aminopiperidine-1-carboxylate (4.44 g, 22.21 mmol) in toluene (40 mL) at room temperature was added KO^{t}Bu (6.23 g, 55.53 mmol) and rac-BINAP (2.304 g, 3.702 mmol). The reaction mixture was degassed with nitrogen for 5 min and followed by addition of Pd₂(dba)₃ (1.69 g, 1.851 mmol). The reaction mixture was stirred at 110° C. for 16 h before it was allowed to cool. The reaction mixture was diluted with water (150 mL) and extracted with ethyl acetate (2×300 mL). The combined organic layer was washed with brine (200 mL), dried over Na₂SO₄, and concentrated under reduced pressure. The residue was purified by Combi-flash using 30% EtOAc in hexane to afford tert-butyl (R)-3-((3-chloro-4-methylpyridin-2-yl)amino)piperidine-1-carboxylate (2.8 g, 46%) as an off-white solid. LC-MS: [M+H⁺]=326.35.

### Step 2:

To a solution of tert-butyl (R)-3-((3-chloro-4-methylpyridin-2-yl)amino)piperidine-1-carboxylate (1.3 g, 3.95 mmol) in THF (10 mL) at 0° C. was added 1.5 M LiHMDS in THF (7 mL, 11.85 mmol). The reaction mixture was stirred for 10 min and followed by addition of 4-(3H-[1,2,3]triazolo[4,5-b]pyridin-3-yl)-2-fluorobenzoyl chloride (1.31 g, 4.74 mmol) in THF (10 mL). The reaction mixture was stirred at room temperature for 2 h before it was diluted with water (30 mL) and extracted with EtOAc (2×10 mL). The combined organic layer was washed with brine, dried over Na₂SO₄, and concentrated. The residue was purified by Combi-flash using 55% EtOAc in hexane and then was further purified by prep-HPLC (Conditions: X-SELECT CSH, Mobile phase A: 0.1% formic acid in water, Mobile phase B: 100% MeCN, Gradient: Time (min)/% B: 0/70, 10/80, Flow: 15 mL/min, Dilution: MeCN+H₂O+THF). The pure fractions were combined and dried by lyophilization to afford tert-butyl (R)-3-(4-(3H-[1,2,3]triazolo[4,5-b]pyridin-3-yl)-N-(3-chloro-4-methylpyridin-2-yl)-2-fluorobenzamido)piperidine-1-carboxylate (800 mg, 1.3 mmol, 23%) as an off-white solid. LC-MS: [M−56]=510.20.

### Step 3:

To a solution of tert-butyl (R)-3-(4-(3H-[1,2,3]triazolo[4,5-b]pyridin-3-yl)-N-(3-chloro-4-methylpyridin-2-yl)-2-fluorobenzamido)piperidine-1-carboxylate (150 mg, 0.265 mmol) in EtOH (5 mL) and water (0.2 mL) at room temperature was added (E)-tert-butyldimethyl ((4-(4,4,5,5-tetramethyl-1,3,2-dioxaborolan-2-yl)but-3-en-1-yl)oxy)silane (99 mg, 0.318 mmol) and K₂CO₃ (109 mg, 0.795 mmol). The reaction mixture was purged with nitrogen for 5 min and followed by addition of XphosPdG2 (20 mg, 0.0265 mmol) and purged with nitrogen for 2 min. The reaction was stirred at 80° C. for 16 h before it was cooled and concentrated. The residue was diluted with water (5 mL) and extracted with EtOAc (2×10 mL). The combined organic layer was washed with brine, dried over Na₂SO₄, and concentrated. The residue was purified by Combi-flash using 55% EtOAc in hexane to afford tert-butyl (R,E)-3-(4-(3H-[1,2,3]triazolo[4,5-b]pyridin-3-yl)-N-(3-(4-((tert-butyldimethylsilyl)oxy)but-1-en-1-yl)-4-methylpyridin-2-yl)-2-fluorobenzamido)piperidine-1-carboxylate (210 mg, impure) as a brown solid. LC-MS: [M+H⁺]=716.30.

### Step 4:

To a solution of tert-butyl (R,E)-3-(4-(3H-[1,2,3]triazolo[4,5-b]pyridin-3-yl)-N-(3-(4-((tertbutyldimethylsilyl)oxy)but-1-en-1-yl)-4-methylpyridin-2-yl)-2 fluorobenzamido)piperidine-1-carboxylate (390 mg, 0.544 mmol) in THF (20 mL) at room temperature was added a solution of TBAF in THF (1.9 mL, 1.634 mmol) and was stirred for 4 h. The reaction mixture was diluted with water (20 mL) and extracted with EtOAc (2×50 mL). The combined organic layer was washed with brine, dried over Na₂SO₄, and concentrated under reduced pressure. The residue was purified by Combi-flash using 90% EtOAc in hexane which was further purified by prep-HPLC (Conditions: Kinetex C-18 (250×21.2×5 μm), Mobile phase A: 0.1% formic acid in water, Mobile phase B: 100% MeCN, Gradient: Time (min)/% B: 0/40, 10/75, Flow: 15 mL/min, Dilution: MeCN+H₂O+THF). The pure fractions were combined and dried by lyophilization to afford tert-butyl (R,E)-3-(4-(3H-[1,2,3]triazolo[4,5-b]pyridin-3-yl)-2-fluoro-N-(3-(4-hydroxybut-1-en-1-yl)-4-methylpyridin-2-yl)benzamido)piperidine-1-carboxylate formate salt (35 mg) as an off white solid. LC-MS: [M+H⁺]=602.25.

### Step 5:

The title compound was obtained as an off-white solid by deprotection Method A used in Example 97. LC-MS: [M+H⁺]=502.20. ¹H NMR (CD₃OD, 400 MHZ): δ 8.71 (d, J=4.4 Hz, 1H), 8.45 (d, J=8.4 Hz, 1H), 8.19 (d, J=4.8 Hz, 1H), 8.13-8.08 (m, 2H), 8.01-7.95 (m, 1H), 7.48-7.45 (m, 1H), 7.33-7.19 (m, 1H), 7.11-7.08 (m, 1H), 6.14 (d, J=16.4 Hz, 1H), 5.85-5.68 (m, 1H), 3.71-3.54 (m, 4H), 2.83-2.77 (m, 1H), 2.47-2.42 (m, 3H), 2.21-2.17 (m, 1H), 2.12 (s, 3H), 1.95-1.78 (m, 3H), 1.47-1.40 (m, 1H).

### Synthesis of (R)-4-(5-(1H-pyrazol-3-yl)-3H-[1,2,3]triazolo[4,5-b]pyridin-3-yl)-2-fluoro-N-(8-methylisoquinolin-1-yl)-N-(piperidin-3-yl)benzamide (Example 138)

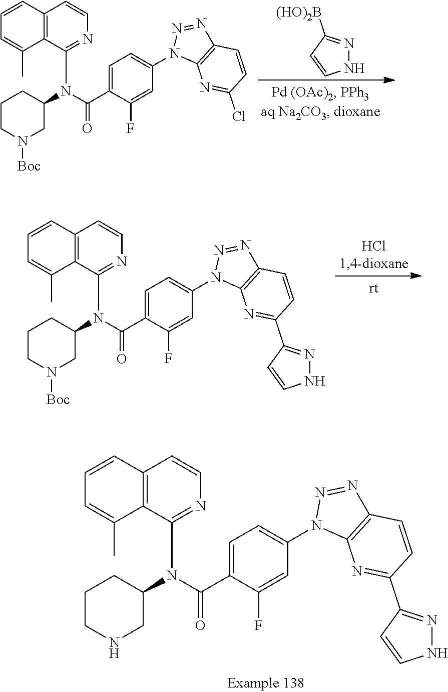 [SMILES: Cc1cccc2ccnc(N(C(=O)c3ccc(-n4nnc5ccc(Cl)nc54)cc3F)[C@@H]3CCCN(C(=O)OC(C)(C)C)C3)c12 ; OB(O)c1cc[nH]n1 ; Cc1cccc2ccnc(N(C(=O)c3ccc(-n4nnc5ccc(-c6cc[nH]n6)nc54)cc3F)[C@@H]3CCCN(C(=O)OC(C)(C)C)C3)c12 ; Cc1cccc2ccnc(N(C(=O)c3ccc(-n4nnc5ccc(-c6cc[nH]n6)nc54)cc3F)[C@@H]3CCCNC3)c12]

### Step 1:

To a solution of tert-butyl (R)-3-(4-(5-chloro-3H-[1,2,3]triazolo[4,5-b]pyridin-3-yl)-2-fluoro-N-(8-methylisoquinolin-1-yl)benzamido)piperidine-1-carboxylate (200 mg, 0.325 mmol) in 1,4-dioxane (5 mL) and H₂O (2 mL) at room temperature was added (1H-pyrazol-3-yl)boronic acid (73 mg, 0.650 mmol), Na₂CO₃ (109.2 mg, 0.975 mmol) and PPh₃ (51 mg, 0.195 mmol). The reaction mixture was purged with nitrogen for 10 min and followed by addition of Pd(OAc)₂ (7.2 mg, 0.0325 mmol). The reaction mixture was stirred at 90° C. for 16 h before it was cooled. The reaction mixture was diluted with EtOAc (50 mL) and filtered through celite. The filtrate was washed with water (20 mL). The organic layer was dried over Na₂SO₄ and concentrated under reduced pressure. The residue was purified by Combi-flash using 100% EtOAc in hexane to afford tert-butyl (R)-3-(4-(5-(1H-pyrazol-3-yl)-3H-[1,2,3]triazolo[4,5-b]pyridin-3-yl)-2-fluoro-N-(8-methylisoquinolin-1-yl)benzamido)piperidine-1-carboxylate that was further purified by Prep-HPLC (Conditions: Luna OMEGA C-18 (250×21.2×5 μm), Mobile phase A: 0.1% formic acid in water (100%), Mobile phase B: 100% MeCN, Gradient: Time (min)/% B: 0/70, 10/90, Flow: 15 mL/min, Dilution: MeCN+H₂O+THF). Pure fractions were combined and dried by lyophilization to afford tert-butyl (R)-3-(4-(5-(1H-pyrazol-3-yl)-3H-[1,2,3]triazolo[4,5-b]pyridin-3-yl)-2-fluoro-N-(8-methylisoquinolin-1-yl)benzamido)piperidine-1-carboxylate (20 mg) as an white solid. LC-MS: [M+H⁺]=648.30.

### Step 2:

The title compound hydrochloride salt was obtained as a white solid by deprotection Method A used in Example 97. LC-MS: [M+H⁺]=548.80. ¹H NMR (CD₃OD, 400 MHz): δ 8.66-8.61 (m, 2H), 8.31-8.25 (m, 2H), 8.13-8.11 (m, 1H), 7.95-7.88 (m, 2H), 7.82-7.80 (m, 1H), 7.67-7.64 (m, 2H), 7.31-7.27 (m, 1H), 7.19 (s, 1H), 5.27-5.13 (m, 1H), 4.23-4.20 (m, 1H), 4.05-3.99 (m, 1H), 3.65-3.57 (m, 1H), 3.30-3.21 (m, 1H), 3.09 (s, 3H), 3.08-2.98 (m, 1H), 2.22-2.02 (m, 2H), 1.82-1.72 (m, 1H).

### Synthesis of (R)-3-(3-fluoro-4-((8-methylisoquinolin-1-yl)(piperidin-3-yl)carbamoyl)phenyl)-N-methyl-3H-[1,2,3]triazolo[4,5-b]pyridine-5-carboxamide (Example 139)

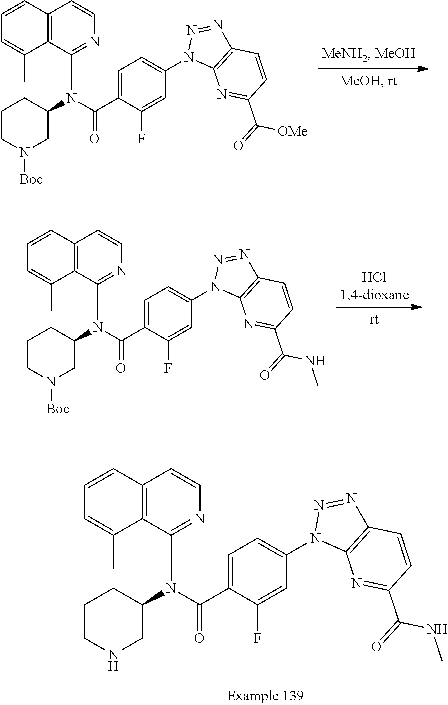 [SMILES: COC(=O)c1ccc2nnn(-c3ccc(C(=O)N(c4nccc5cccc(C)c45)[C@@H]4CCCN(C(=O)OC(C)(C)C)C4)c(F)c3)c2n1 ; CNC(=O)c1ccc2nnn(-c3ccc(C(=O)N(c4nccc5cccc(C)c45)[C@@H]4CCCN(C(=O)OC(C)(C)C)C4)c(F)c3)c2n1 ; CNC(=O)c1ccc2nnn(-c3ccc(C(=O)N(c4nccc5cccc(C)c45)[C@@H]4CCCNC4)c(F)c3)c2n1]

### Step 1:

To a solution of methyl (R)-3-(4-((1-(tert-butoxycarbonyl)piperidin-3-yl)(8-methylisoquinolin-1-yl)carbamoyl)-3-fluorophenyl)-3H-[1,2,3]triazolo[4,5-b]pyridine-5 carboxylate (25 mg, 0.0390 mmol) in MeOH (5 mL) at 0° C. was added MeNH₂ (0.1 mL). The reaction was then stirred at 25° C. for 16 h. The reaction mixture was concentrated under reduced pressure to afford crude tert-butyl (R)-3-(2-fluoro-4-(5-(methylcarbamoyl)-3H-[1,2,3]triazolo[4,5-b]pyridin-3-yl)-N-(8-methylisoquinolin-1-yl)benzamido)piperidine-1-carboxylate (20 mg) as an off-white solid. LC-MS: [M+H⁺]=639.10.

### Step 2:

To a solution of tert-butyl (R)-3-(2-fluoro-4-(5-(methylcarbamoyl)-3H-[1,2,3]triazolo[4,5-b]pyridin-3-yl)-N-(8-methylisoquinolin-1-yl)benzamido)piperidine-1-carboxylate (25 mg, 0.0391 mmol) in DCM (2 mL) at 0° C. was added 4M HCl in 1,4-dioxane (5 mL) and was then stirred at room temperature for 2 h. The reaction mixture was concentrated and dried by lyophilization afford (R)-3-(3-fluoro-4-((8-methylisoquinolin-1-yl)(piperidin-3-yl)carbamoyl)phenyl)-N-methyl-3H-[1,2,3]triazolo[4,5-b]pyridine-5-carboxamide HCl which was purified by Prep-HPLC (Conditions: Luna OMEGA C-18 (250×21.2×5 μm), Mobile phase A: 0.1% formic acid in water, Mobile phase B: 100% MeCN, Gradient: Time (min)/% B: 0/70, 10/95, Flow: 15 mL/min, Dilution: MeCN+H₂O+THF). The pure fractions were combined and dried by lyophilization to afford (R)-3-(3-fluoro-4-((8-methylisoquinolin-1-yl)(piperidin-3-yl)carbamoyl)phenyl)-N-methyl-3H-[1,2,3]triazolo[4,5-b]pyridine-5-carboxamide formic acid salt (7 mg) as an off-white solid. LC-MS: [M+H⁺]=539.25. ¹H NMR (CD₃OD, 400 MHZ): δ 8.64 (d, J=8.4 Hz, 1H), 8.45-8.41 (m, 1H), 8.25 (d, J=8.8 Hz, 1H), 8.03-7.95 (m, 2H), 7.73-7.63 (m, 2H), 7.51-7.50 (m, 2H), 7.18-7.14 (m, 1H), 3.92-3.89 (m, 1H), 3.62-3.57 (m, 1H), 3.22-3.12 (m, 1H), 3.01 (s, 3H), 2.89 (s, 3H), 2.76-2.62 (m, 2H), 1.88-1.79 (m, 3H), 1.48-1.35 (m, 1H).

### Synthesis of methyl (R)-3-(3-fluoro-4-((8-methylisoquinolin-1-yl)(piperidin-3-yl)carbamoyl)phenyl)-3H-[1,2,3]triazolo[4,5-b]pyridine-5-carboxylate (Example 140)

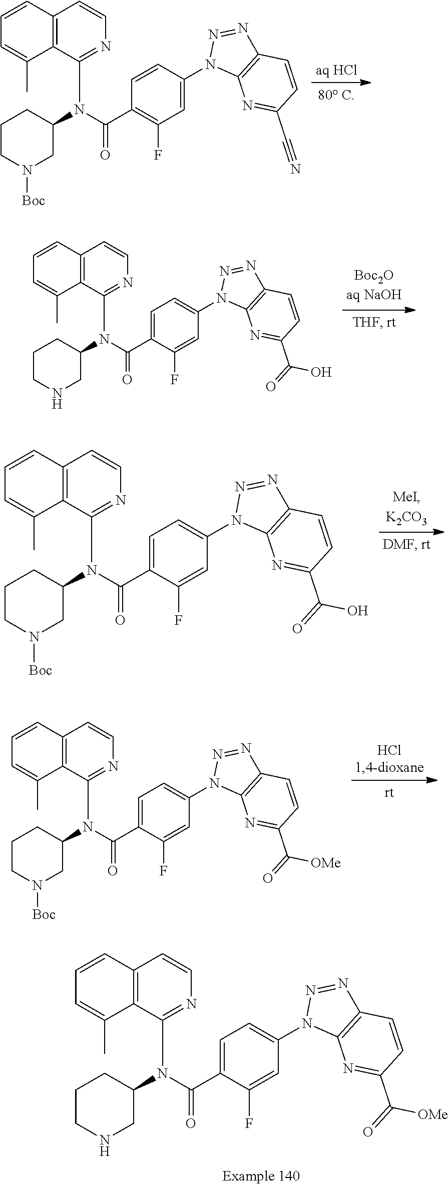 [SMILES: Cc1cccc2ccnc(N(C(=O)c3ccc(-n4nnc5ccc(C#N)nc54)cc3F)[C@@H]3CCCN(C(=O)OC(C)(C)C)C3)c12 ; Cc1cccc2ccnc(N(C(=O)c3ccc(-n4nnc5ccc(C(=O)O)nc54)cc3F)[C@@H]3CCCNC3)c12 ; Cc1cccc2ccnc(N(C(=O)c3ccc(-n4nnc5ccc(C(=O)O)nc54)cc3F)[C@@H]3CCCN(C(=O)OC(C)(C)C)C3)c12 ; COC(=O)c1ccc2nnn(-c3ccc(C(=O)N(c4nccc5cccc(C)c45)[C@@H]4CCCN(C(=O)OC(C)(C)C)C4)c(F)c3)c2n1 ; COC(=O)c1ccc2nnn(-c3ccc(C(=O)N(c4nccc5cccc(C)c45)[C@@H]4CCCNC4)c(F)c3)c2n1]

### Step 1:

To a solution of tert-butyl (R)-3-(4-(5-cyano-3H-[1,2,3]triazolo[4,5-b]pyridin-3-yl)-2-fluoro-N-(8-methylisoquinolin-1-yl)benzamido)piperidine-1-carboxylate (110 mg, 0.181 mmol) in water (5 ml) was added 5N aqueous HCl (2 mL) at 0° C. The reaction mixture was then stirred at 90° C. for 16 h. The reaction mixture was concentrated and dried to afford crude (R)-3-(3-fluoro-4-((8-methylisoquinolin-1-yl)(piperidin-3-yl)carbamoyl)phenyl)-3H-[1,2,3]triazolo[4,5-b]pyridine-5-carboxylic acid (100 mg) as a white solid. LC-MS: [M−H+]=526.75.

### Step 2:

To a solution of crude (R)-3-(3-fluoro-4-((8-methylisoquinolin-1-yl)(piperidin-3-yl)carbamoyl)phenyl)-3H-[1,2,3]triazolo[4,5-b]pyridine-5-carboxylic acid (80 mg, 0.152 mmol) in THF (2 mL) and water (2 mL) was added NaHCO₃ (25.61 mg, 0.3047 mmol) at room temperature. Then di-tert-butyl decarbonate (69.4 mg, 0.3047 mmol) was added to reaction mixture and was stirred at room temperature for 5 h. The reaction mixture was partitioned between ethyl acetate (50 ml) and water (2×20 mL). The organic layer was dried by Na₂SO₄ and concentrated under reduced pressure to afford R)-3-(4-((1-(tert-butoxycarbonyl)piperidin-3-yl)(8-methylisoquinolin-1-yl)carbamoyl)-3-fluorophenyl)-3H-[1,2,3]triazolo[4,5-b]pyridine-5-carboxylic acid (90 mg) as a white solid. LC-MS: [M−H⁺]=626.55.

### Step 3:

To a solution of (R)-3-(4-((1-(tert-butoxycarbonyl)piperidin-3-yl)(8-methylisoquinolin-1-yl)carbamoyl)-3-fluorophenyl)-3H-[1,2,3]triazolo[4,5-b]pyridine-5-carboxylic acid (100 mg, 0.1599 mmol) in DMF (5 mL) at 0° C. was added K₂CO₃ (66.3 mg, 0.479 mmol). The reaction mixture was stirred for 10 min and followed by addition of iodomethane (45.3 mg, 0.3198 mmol) at 0° C. The reaction mixture was stirred at 100° C. for 16 h before it was cooled. The reaction mixture was diluted with water and extracted with EtOAc (2×50 mL). The combined organic layer was washed with brine, dried over Na₂SO₄, and concentrated under reduced pressure. The residue was purified by Prep-HPLC (Conditions: X-SELECT, Mobile phase A: 0.1% formic acid in water (100%), Mobile phase B: 100% MeCN, Gradient: Time (min)/% B: 0/80, 10/95, Flow: 15 mL/min, Dilution: MeCN+H₂O+THF). Pure fractions were combined and dried by lyophilization to afford tert-butyl (R)-3-(N-(8-methylisoquinolin-1-yl)-3-phenyl-5,6,7,8-tetrahydro-[1,2,4]triazolo[4,3-a]pyrazine-7-carboxamido)piperidine-1-carboxylate (55 mg) as an off-white solid. LC-MS: [M−H⁺]=640.45.

### Step 4:

The title compound hydrochloride salt was obtained as a white solid by deprotection Method A used in Example 97. LC-MS: [M+H⁺]=540.30. ¹H NMR (CD₃OD, 400 MHZ): δ 8.66 (d, J=8.8 Hz, 1H), 8.47-8.44 (m, 1H), 8.24 (d, J=8.8 Hz, 1H), 8.09-8.01 (m, 1H), 7.92-7.89 (m, 1H), 7.76-7.70 (m, 1H), 7.66-7.62 (m, 1H), 7.51-7.48 (m, 2H), 7.15-7.10 (m, 1H), 5.13-5.05 (m, 1H), 4.02 (s, 3H), 3.86-3.83 (m, 1H), 3.67-3.62 (m, 1H), 3.42-3.32 (m, 1H), 3.07-3.00 (m, 1H), 2.88 (s, 3H), 1.98-1.93 (m, 2H), 1.59-1.56 (m, 1H), 1.35-1.28 (m, 1H).

### Synthesis of (R)—N-(8-chloroisoquinolin-1-yl)-2-fluoro-4-iodo-N-(piperidin-3-yl)benzamide (Example 141)

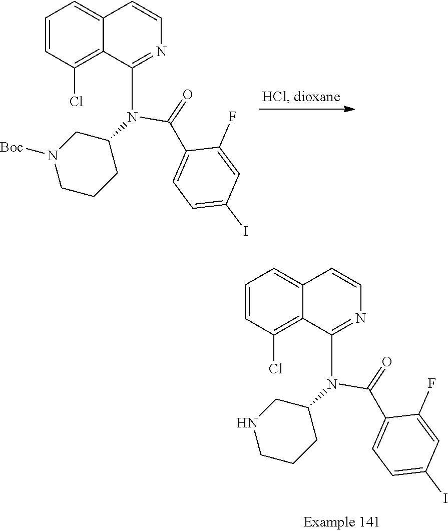

To a solution of tert-butyl (R)-3-(N-(8-chloroisoquinolin-1-yl)-2-fluoro-4-iodobenzamido)piperidine-1-carboxylate (30.00 mg, 49.19 μmol, 1 eq) in dioxane (1 mL) was added HCl/dioxane (4 M, 1 mL) at 20° C. The reaction was stirred at 20° C. for 2 h. The reaction mixture was concentrated to give (R)—N-(8-chloroisoquinolin-1-yl)-2-fluoro-4-iodo-N-(piperidin-3-yl)benzamide hydrochloride salt (17.2 mg, 69%) as a white solid. LCMS (ESI⁺) [M+H⁺]=510.0. ¹H NMR (400 MHZ, DMSO-d₆): δ 0.97-1.26 (m, 1H) 1.71-1.90 (m, 3H) 2.66-2.91 (m, 1H) 3.14-3.51 (m, 2H) 4.72-5.04 (m, 1H) 6.72-6.80 (m, 1H) 7.18 (dd, J=8.13, 1.25 Hz, 1H) 7.29-7.40 (m, 1H) 7.62-7.74 (m, 1H) 7.83 (d, J=7.38 Hz, 1H) 7.92-7.99 (m, 2H) 8.49-8.60 (m, 1H) 8.99-9.37 (m, 2H).

### Synthesis of (R)-4-(5-ethyl-2H-tetrazol-2-yl)-2-fluoro-N-(8-methylisoquinolin-1-yl)-N-(piperidin-3-yl)benzamide (Example 142)

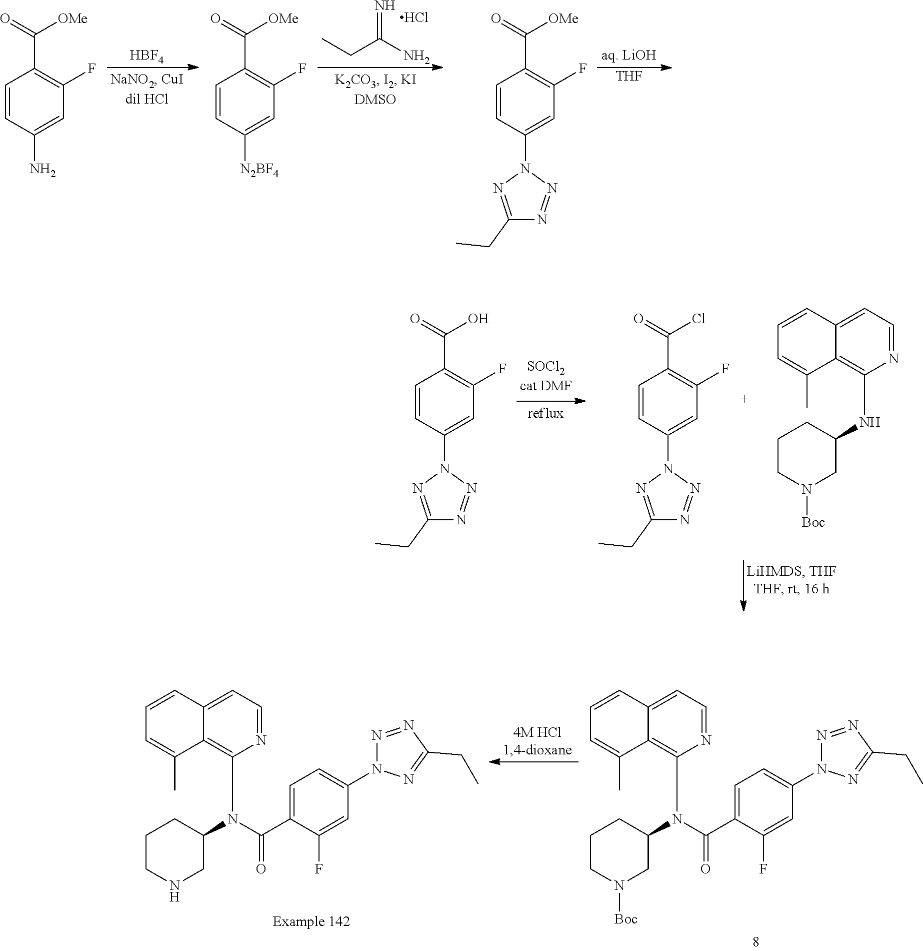

### Step 1:

To a solution of methyl 4-amino-2-fluorobenzoate (10 g, 59.115 mmol) in H₂O (75 mL) at 0° C. were added HBF (48 wt % in water, 18.5 ml, 118.231 mmol) and a solution of NaNO₂ (4.07 g, 59.115 mmol) in H₂O (25 mL) dropwise and stirred for 15 min before the reaction mixture was filtered. The isolated solid was dissolved in cold acetone (200 mL), added Et₂O until no further precipitation is observed. The precipitate was isolated by filtration and dried by vacuum to afford crude methyl (E)-2-fluoro-4-((tetrafluoro-15 boraneyl)diazenyl)benzoate (6 g) as a pale yellow solid.

### Step 2:

To a solution of propionimidamide hydrochloride salt (1.19 g, 11.040 mmol) in DMSO (30 mL) at 0° C. were added K₂CO₃ (3.8 g, 55.202 mmol) and 3-fluoro-4(methoxycarbonyl)benzenediazonium tetrafluoroborate (2.0 g, 11.040 mmol) from the last step. The reaction mixture was stirred at 25° C. for 1 h before it was cooled to 0° C. and followed by a solution of I₂ (3.36 g, 13.248 mmol) and KI (2.74 g, 16.560 mmol) in dry DMSO (10 mL). The resulting reaction mixture stirred at room temperature for 1 h before it was quenched with 5% aqueous Na₂S₂O₃ (35 mL) and extracted with EtOAc (2×50 mL). The combined organic layer was washed with brine, dried over Na₂SO₄, and concentrated under reduced pressure. The crude product was purified by Combi-flash using 40% EtOAc in hexane to afford methyl 4-(5-ethyl-2H-tetrazol-2-yl)-2-fluorobenzoate (500 mg, 18%) as a pale-yellow solid. LC-MS: [M+H⁺]=251.10.

### Step 3:

To a solution of methyl 4-(5-ethyl-2H-tetrazol-2-yl)-2-fluorobenzoate (500 mg, 2.116 mmol) in THF (10 mL) and H₂O (10 mL) at room temperature was added LiOH·H₂O (440 mg, 10.583 mmol) and stirred for 6 h. The reaction mixture was concentrated under reduced pressure. The crude residue was diluted with water and acidified with 2N aq. HCl. The precipitate was isolated by filtration and dried by vacuum to afford 4-(5-ethyl-2H-tetrazol-2-yl)-2-fluorobenzoic acid (350 mg, 74%) as an off-white solid. LC-MS: [M+H⁺]=237.30.

### Step 4:

To a solution of 4-(5-ethyl-2H-tetrazol-2-yl)-2-fluorobenzoic acid (200 mg, 0.846 mmol) in SOCl₂ (10 mL) at 0° C. was added DMF (0.1 mL). The reaction mixture stirred at 80° C. for 16 h before it was concentrated under reduced pressure to afford crude 4-(5-ethyl-2H-tetrazol-2-yl)-2-fluorobenzoyl chloride (220 mg, crude) as a solid.

### Step 5:

To a solution of tert-butyl (R)-3-((8-methylisoquinolin-1-yl)amino)piperidine-1-carboxylate (200 mg, 0.585 mmol) in THF (10 mL) at 0° C. was added 1M LiHMDS in THF (1.7 mL, 1.759 mmol) and stirred for 10 min. To this solution at 0° C., crude 4-(5-ethyl-2H-tetrazol-2-yl)-2-fluorobenzoyl chloride (170 mg, 0.702 mmol) was added and then stirred for 20 min. The reaction mixture was quenched with ice cold water and extracted with EtOAc (2×50 mL). The combined organic layer was washed with brine, dried over Na₂SO₄, and concentrated under reduced pressure. The crude product was first purified by combi-flash using 40% EtOAc in hexane to and then further purified by Prep-HPLC [Conditions: Luna OMEGA C-18 (250×21.2×5 μm), Mobile phase A: 0.1% formic acid in H₂O, Mobile phase B: 100% MeCN, Gradient: T/% B: 0/75, 10/95, Flow: 15 mL/min, Dilution: MeCN/H₂O/THF]. Pure fractions were dried by lyophilization to afford (R)-3-(4-(5-ethyl-2H-tetrazol-2-yl)-2-fluoro-N-(8-methylisoquinolin-1-yl)benzamido)piperidine-1-carboxylate (40 mg, 12%) as a pale-yellow solid.

### Step 6:

The title compound hydrochloride salt was obtained as an off-white solid by deprotection Method A used in Example 97. LC-MS: [M+H⁺]=460.2. ¹H NMR (CD₃OD, 400 MHZ): δ 8.49-8.46 (m, 1H), 7.79-7.76 (m, 1H), 7.70-7.69 (m, 1H), 7.63-7.57 (m, 1H), 7.58-7.50 (m, 3H), 7.14-7.10 (m, 1H), 5.14-5.08 (m, 1H), 4.0-4.04 (m, 1H), 3.90-3.84 (m, 1H), 3.46-3.43 (m, 1H), 3.10-3.00 (m, 1H), 2.96-2.89 (m, 2H), 2.73 (s, 3H), 2.00-1.97 (m, 3H), 1.66-1.63 (m, 1H), 1.40-1.36 (m, 3H).

### Synthesis of (R)-4-(3H-[1,2,3]triazolo[4,5-b]pyridin-3-yl)-N-(6-bromoisoquinolin-1-yl)-2-fluoro-N-(piperidin-3-yl)benzamide (Example 143)

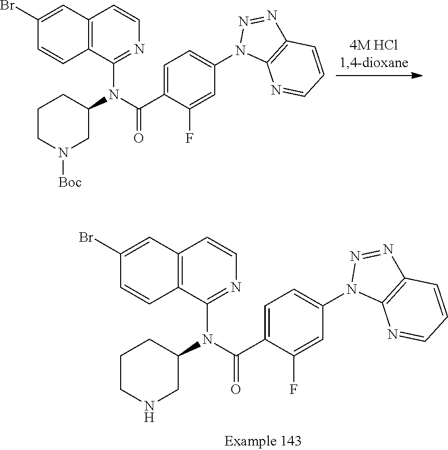 [SMILES: CC(C)(C)OC(=O)N1CCC[C@@H](N(C(=O)c2ccc(-n3nnc4cccnc43)cc2F)c2nccc3cc(Br)ccc23)C1 ; O=C(c1ccc(-n2nnc3cccnc32)cc1F)N(c1nccc2cc(Br)ccc12)[C@@H]1CCCNC1]

To a solution of tert-butyl (R)-3-(4-(3H-[1,2,3]triazolo[4,5-b]pyridin-3-yl)-N-(6-bromoisoquinolin-1-yl)-2-fluorobenzamido)piperidine-1-carboxylate (50 mg, 0.0775 mmol) in DCM (2 mL) at 0° C. was added 4M HCl in 1,4-dioxane (5 mL). The reaction mixture was stirred at room temperature for 2 h. The reaction was concentrated under reduced pressure and dried by lyophilization to afford (R)-4-(3H-[1,2,3]triazolo[4,5-b]pyridin-3-yl)-N-(6-bromoisoquinolin-1-yl)-2-fluoro-N-(piperidin-3-yl)benzamide hydrochloride salt as an off-white solid. LC-MS: [M+H⁺]=547.8. ¹H NMR (CD₃OD, 400 MHZ): δ 8.75 (d, J=3.6 Hz, 1H), 8.50-8.44 (m, 2H), 8.13-7.97 (m, 3H), 7.97-7.83 (m, 2H), 7.73-7.71 (m, 1H), 7.54-7.51 (m, 1H), 7.34-7.30 (m, 1H), 5.29-5.23 (m, 1H), 3.97-3.94 (m, 1H), 3.69-3.66 (m, 1H), 3.48-3.30 (m, 1H), 2.89-2.80 (m, 1H), 2.00-1.87 (m, 3H), 1.43-1.25 (m, 1H).

### Synthesis of (R)-4-(3H-[1,2,3]triazolo[4,5-b]pyridin-3-yl)-2-fluoro-N-(6-fluoroisoquinolin-1-yl)-N-(piperidin-3-yl)benzamide (Example 144)

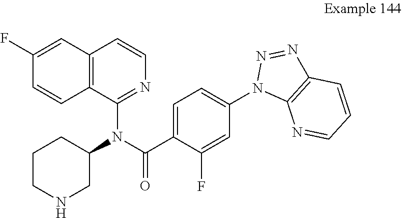 [SMILES: O=C(c1ccc(-n2nnc3cccnc32)cc1F)N(c1nccc2cc(F)ccc12)[C@@H]1CCCNC1]

Example 144 was prepared according to the procedure described for Example 143 by substituting 6-bromo-1-chloroisoquinoline with 6-fluoro-1-chloroisoquinoline, provided the title compound as an off-white solid. LC-MS: [M+H⁺]=486.9. ¹H NMR (CD₃OD, 400 MHz): δ 8.78-8.72 (m, 1H), 8.51-8.41 (m, 2H), 8.34-8.48 (m, 1H), 8.06-8.03 (m, 1H), 7.97-7.91 (m, 1H), 7.77-7.76 (m, 1H), 7.63-7.53 (m, 3H), 7.34-7.30 (m, 1H), 5.30-5.27 (m, 1H), 3.98-3.95 (m, 1H), 3.73-3.67 (m, 1H), 2.92-2.88 (m, 1H), 2.03-2.00 (m, 1H), 1.99-1.91 (m, 2H), 1.37-1.23 (m, 2H).

### Synthesis of (R)-4-(3H-[1,2,3]triazolo[4,5-b]pyridin-3-yl)-2-fluoro-N-(6-methoxyisoquinolin-1-yl)-N-(piperidin-3-yl)benzamide (Example 145)

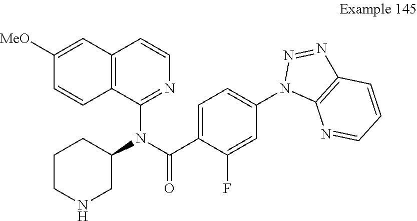 [SMILES: COc1ccc2c(N(C(=O)c3ccc(-n4nnc5cccnc54)cc3F)[C@@H]3CCCNC3)nccc2c1]

Example 145 was prepared according to the procedure described for Example 143 by substituting 6-bromo-1-chloroisoquinoline with 6-methoxy-1-chloroisoquinoline, provided the title compound as an off-white solid. LC-MS: [M+H⁺]=498.9. ¹H NMR (CD₃OD, 400 MHz): δ 8.76-8.75 (m, 1H), 8.51-8.49 (m, 1H), 8.37-8.35 (m, 1H), 8.16-7.93 (m, 2H), 7.95-7.93 (m, 1H), 7.81-7.71 (m, 1H), 7.54-7.51 (m, 1H), 7.46-7.34 (m, 2H), 7.31-7.22 (m, 2H), 5.25-5.20 (m, 1H), 3.96-3.92 (m, 1H), 3.87 (s, 3H), 3.71-3.65 (m, 1H), 2.94-2.84 (m, 1H), 2.13-1.86 (m, 3H), 1.37-1.28 (m, 1H), 0.96-0.89 (m, 1H).

### Synthesis of (R)-4-(3H-[1,2,3]triazolo[4,5-b]pyridin-3-yl)-2-fluoro-N-(6-(hydroxymethyl)isoquinolin-1-yl)-N-(piperidin-3-yl)benzamide (Example 146)

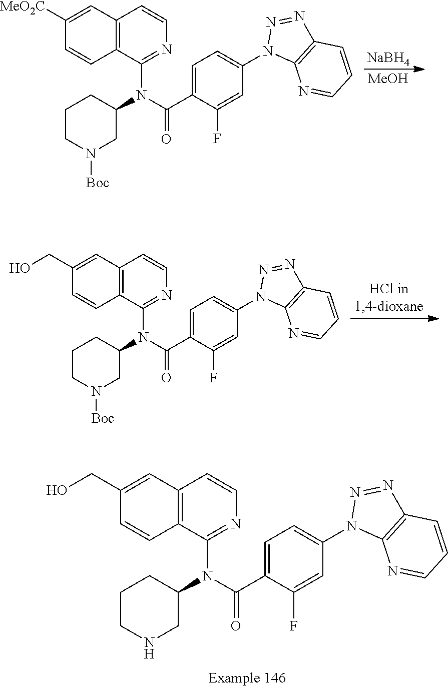

### Step 1:

To a stirred solution of methyl (R)-1-(4-(3H-[1,2,3]triazolo[4,5-b]pyridin-3-yl)-N-(1-(tert-butoxycarbonyl)piperidin-3-yl)-2-fluorobenzamido)isoquinoline-6-carboxylate (40 mg, 0.0639 mmol) in MeOH (5 mL) at 0° C. was added NaBH₄ (7.2 g, 0.1919 mmol). The reaction mixture stirred at room temperature for 16 h before it was quenched with ice cold water and extracted with EtOAc (2×100 mL). The combined organic layer was washed with brine, dried over Na₂SO₄, and concentrated under reduced pressure. The crude product was first purified by Combi-flash using 30% EtOAc in hexane and then further purified by Prep-HPLC [Conditions: Column: Luna OMEGA C-18 (250×21.2×5 μm), Mobile phase A: 0.1% formic acid in water (100%), Mobile phase B: 100% MeCN, Gradient: T/% B: 0/65, 10/80, Flow: 15 mL/min, Dilution: MeCN/H₂O/THF]. Combined pure fractions were and dried by lyophilization to afford tert-butyl (R)-3-(4-(3H-[1,2,3]triazolo[4,5-b]pyridin-3-yl)-2-fluoro-N-(6-(hydroxymethyl)isoquinolin-1-yl)benzamido)piperidine-1-carboxylate (300 mg) as white solid. LC-MS: [M+H⁺]=598.2.

The title compound hydrochloride salt was obtained as an off-white solid by deprotection Method A used in Example 97. LC-MS: [M+H⁺]=498.2. ¹H NMR (CD₃OD, 400 MHZ): δ 8.92-8.91 (m, 1H), 8.67-8.58 (m, 2H), 8.33-8.31 (m, 1H), 8.22-8.19 (m, 1H), 8.11-8.06 (m, 1H), 7.96-7.87 (m, 3H), 7.71-7.68 (m, 1H), 7.50-7.46 (m, 1H), 5.47-5.43 (m, 1H), 4.75-4.68 (m, 1H), 4.65 (s, 2H), 3.38-3.36 (m, 1H), 3.19-3.16 (m, 2H), 1.93-1.88 (m, 1H), 1.78-1.75 (m, 2H), 1.10-0.80 (m, 1H).

### Synthesis of (R)-4-(3H-[1,2,3]triazolo[4,5-b]pyridin-3-yl)-2-fluoro-N-(6-cyanoisoquinolin-1-yl)-N-(piperidin-3-yl)benzamide (Example 147)

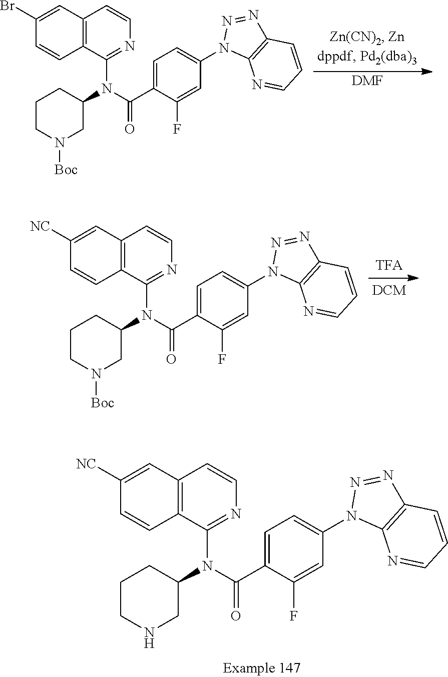

### Step 1:

To a solution tert-butyl (R)-3-(4-(3H-[1,2,3]triazolo[4,5-b]pyridin-3-yl)-N-(6-bromoisoquinolin-1-yl)-2-fluorobenzamido)piperidine-1-carboxylate (300 mg, 0.465 mmol) in DMF (10 mL) were added Zn(CN)₂ (164.9 mg, 0.930 mmol), Zn (3.6 mg, 0.0558 mmol) and 1,1′-bis(diphenylphosphino) ferrocene (10.3 mg, 0.0186 mmol) at rt. The reaction mixture was purged with nitrogen balloon for 5 min before Pd₂(dba)₃ (17 mg, 0.0186 mmol) was added. The reaction mixture was stirred at 120° C. for 5 h before it was allowed to cool. The reaction mixture diluted with EtOAc (100 mL) and filtered through celite. The filtrate was washed with water (80 mL). The organic layer was washed with brine, dried over Na₂SO₄, and concentrated under reduced pressure. The crude product was purified first by combi-flash using 80% EtOAc in hexane to provide the slightly impure product (110 mg). This material was further purified by Prep-HPLC [Conditions: X-SELECT CSH column, Mobile phase A: 0.1% formic acid in water (100%), Mobile phase B: 100% MeCN, Gradient: T/% B: 1/65, 10/85, Flow: 15 mL/min, Dilution: MeCN/H₂O/THF]. Pure fractions were dried by lyophilization for 2 days to afford tert-butyl (R)-3-(4-(3H-[1,2,3]triazolo[4,5-b]pyridin-3-yl)-N-(6-cyanoisoquinolin-1-yl)-2-fluorobenzamido)piperidine-1-carboxylate (55 mg, 50%) as a white solid. LC-MS: [M+H⁺]=537.20.

### Step 2:

To a solution of tert-butyl (R)-3-(4-(3H-[1,2,3]triazolo[4,5-b]pyridin-3-yl)-N-(6-cyanoisoquinolin-1-yl)-2-fluorobenzamido)piperidine-1-carboxylate (50 mg, 0.0844 mmol) in DCM (5 mL) at 0° C. was added TFA (2 mL). The reaction mixture was stirred at rt for 2 h before it was concentrated under reduced pressure to afford crude (R)-4-(3H-[1,2,3]triazolo[4,5-b]pyridin-3-yl)-N-(6-cyanoisoquinolin-1-yl)-2-fluoro-N-(piperidin-3-yl)benzamide TFA salt. The crude product was further purified by Prep-HPLC [Conditions: Luna OMEGA C-18 (250×21.2×5 μm), Mobile phase A: 0.1% formic acid in water (100%), Mobile phase B: 100% MeCN, Gradient: T/% B: 0/20, 10/50, Flow: 15 mL/min, Dilution: MeCN/H₂O/THF]. Pure fractions were dried by lyophilization to afford (R)-4-(3H-[1,2,3]triazolo[4,5-b]pyridin-3-yl)-N-(6-cyanoisoquinolin-1-yl)-2-fluoro-N-(piperidin-3-yl)benzamide TFA salt (35 mg, 77%) as an off white solid. LC-MS: [M+H⁺]=493.90. ¹H NMR (CD₃OD, 400 MHZ): δ 8.75-8.74 (m, 1H), 8.63-8.57 (m, 1H), 8.50 (d, J=8.0 Hz, 1H), 8.41-8.37 (m, 2H), 8.03 (d, J=10.8 Hz, 1H), 7.97-7.91 (m, 2H), 7.89-7.88 (m, 1H), 7.54-7.51 (m, 1H), 7.33-7.29 (m, 1H), 5.30-5.27 (m, 1H), 3.98-3.95 (m, 1H), 3.72-3.67 (m, 1H), 2.89-2.85 (m, 1H), 2.03-1.98 (m, 1H), 1.91-1.90 (m, 2H), 1.31-1.24 (m, 1H).

### Synthesis of (R)-4-(3H-[1,2,3]triazolo[4,5-b]pyridin-3-yl)-N-(7-(3-amino-3-oxopropyl)isoquinolin-1-yl)-2-fluoro-N-(piperidin-3-yl)benzamide (Example 148)

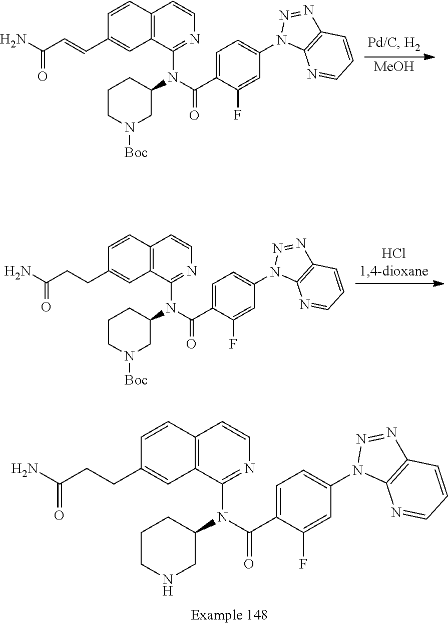 [SMILES: "MeOH" ; CC(C)(C)OC(=O)N1CCC[C@@H](N(C(=O)c2ccc(-n3nnc4cccnc43)cc2F)c2nccc3ccc(/C=C/C(N)=O)cc23)C1 ; CC(C)(C)OC(=O)N1CCC[C@@H](N(C(=O)c2ccc(-n3nnc4cccnc43)cc2F)c2nccc3ccc(CCC(N)=O)cc23)C1 ; NC(=O)CCc1ccc2ccnc(N(C(=O)c3ccc(-n4nnc5cccnc54)cc3F)[C@@H]3CCCNC3)c2c1]

### Step 1:

To a solution of tert-butyl (R,E)-3-(4-(3H-[1,2,3]triazolo[4,5-b]pyridin-3-yl)-N-(7-(3-amino-3-oxoprop-1-en-1-yl)isoquinolin-1-yl)-2-fluorobenzamido)piperidine-1-carboxylate (50 mg, 0.078 mmol) in MeOH (5 mL) at room temperature was added 10% Pd/C (5 mg) and the reaction mixture was stirred under a H₂ balloon for 1 h. The reaction mixture was diluted with methanol and filtrated through a bed of celite. The filtrate was concentrated under reduced pressure. The residue was further purified by Prep-HPLC (Conditions: Column: X-SELECT CSH, Mobile phase A: 0.1% formic acid in water (100%), Mobile phase B: 100% MeCN, Gradient: T/% B: 0/60, 10/95, Flow: 15 mL/min, Dilution: MeCN+H₂O+THF). Pure fractions were dried by lyophilization to afford tert-butyl (R)-3-(4-(3H-[1,2,3]triazolo[4,5-b]pyridin-3-yl)-N-(7-(3-amino-3-oxopropyl)isoquinolin-1-yl)-2-fluorobenzamido)piperidine-1-carboxylate (26 mg, 0.031 mmol, 51%) as an off-white solid. LC-MS: [M-Boc+H⁺]=539.3.

### Step 2:

The title compound hydrochloride salt was obtained as an off-white solid by deprotection Method A used in Example 97. LC-MS: [M+H⁺]=539.20. ¹H NMR (CD₃OD, 400 MHZ): δ 8.82-8.81 (m, 1H), 8.57-8.55 (m, 1H), 8.48-8.43 (m, 1H), 8.12-8.09 (m, 1H), 8.05-8.00 (m, 1H), 7.98-7.95 (m, 1H), 7.90-7.88 (m, 1H), 7.80-7.79 (m, 1H), 7.72-7.70 (m, 1H), 7.61-7.58 (m, 1H), 7.41-7.37 (m, 1H), 5.34-5.28 (m, 1H), 4.05-4.02 (m, 1H), 3.80-3.74 (m, 1H), 3.26-3.22 (m, 3H), 2.97-2.94 (m, 1H), 2.79-2.64 (m, 2H), 2.13-2.10 (m, 1H), 1.99-1.97 (m, 2H), 1.37-1.33 (m, 1H).

### Synthesis of (R,E)-4-(3H-[1,2,3]triazolo[4,5-b]pyridin-3-yl)-N-(7-(3-amino-3-oxoprop-1-en-1-yl)isoquinolin-1-yl)-2-fluoro-N-(piperidin-3-yl)benzamide (Example 149)

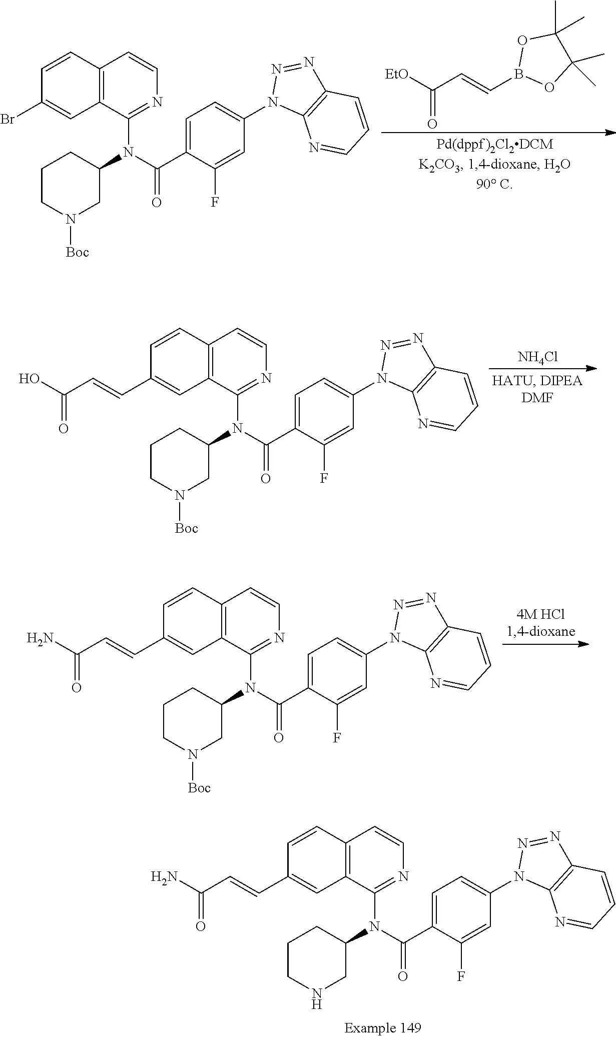 [SMILES: CC(C)(C)OC(=O)N1CCC[C@@H](N(C(=O)c2ccc(-n3nnc4cccnc43)cc2F)c2nccc3ccc(Br)cc23)C1 ; CCOC(=O)/C=C/B1OC(C)(C)C(C)(C)O1 ; CC(C)(C)OC(=O)N1CCC[C@@H](N(C(=O)c2ccc(-n3nnc4cccnc43)cc2F)c2nccc3ccc(/C=C/C(=O)O)cc23)C1 ; "NH4Cl" ; CC(C)(C)OC(=O)N1CCC[C@@H](N(C(=O)c2ccc(-n3nnc4cccnc43)cc2F)c2nccc3ccc(/C=C/C(N)=O)cc23)C1 ; NC(=O)/C=C/c1ccc2ccnc(N(C(=O)c3ccc(-n4nnc5cccnc54)cc3F)[C@@H]3CCCNC3)c2c1]

### Step 1:

To a solution of tert-butyl (R)-3-(4-(3H-[1,2,3]triazolo[4,5-b]pyridin-3-yl)-N-(7-bromoisoquinolin-1-yl)-2-fluorobenzamido)piperidine-1-carboxylate (200 mg, 0.309 mmol) in 1,4-dioxane (5 mL) and water (1 mL) at room temperature were added ethyl (E)-3-(4,4,5,5-tetramethyl-1,3,2-dioxaborolan-2-yl)acrylate (82 mg, 0.362 mmol) and then K₂CO₃ (120 mg, 0.869 mmol). The reaction mixture was degassed with nitrogen for 5 min and followed by addition of Pd(dppf)Cl₂·DCM (11 mg, 0.015 mmol). The reaction was heated to 90° C. and stirred for 16 h, after which it was diluted with water (20 mL) and extracted with EtOAc (2×20 mL). The combined organic layer was washed with brine, dried over Na₂SO₄, and concentrated. The residue was purified by Combi-flash using 65-70% EtOAc in hexane to afford (R,E)-3-(1-(4-(3H-[1,2,3]triazolo[4,5-b]pyridin-3-yl)-N-(1-(tert-butoxycarbonyl)piperidin-3-yl)-2-fluorobenz-amido)isoquinolin-7-yl)acrylic acid (150 mg, 75%) as a yellow syrup. LC-MS: [M+H⁺]=538.1.

### Step 2:

To a solution of (R,E)-3-(1-(4-(3H-[1,2,3]triazolo[4,5-b]pyridin-3-yl)-N-(1-(tert-butoxycarbonyl)piperidin-3-yl)-2-fluorobenzamido)isoquinolin-7-yl)acrylic acid (150 mg, 0.235 mmol) in DMF (5 mL) at room temperature were added ammonium chloride (15 mg, 0.277 mmol), HATU (170 mg, 0.447 mmol), and then DIPEA (0.1 mL, 0.706 mmol). The reaction mixture was stirred at 25° C. for 16 h before it was diluted with water (10 mL) and extracted with EtOAc (2×10 mL). The combined organic layer was dried over Na₂SO₄ and concentrated under reduced pressure. The residue was purified by Prep-HPLC (Conditions: Column: Luna OMEGA C-18 (250×21.2×5 μm), Mobile phase A: 0.1% formic acid in water (100%), Mobile phase B: MeCN (100%), Gradient: T/% B: 0/50, 10/80, Flow: 15 mL/min, Dilution: MeCN+H₂O+THF). Pure fractions were dried by lyophilization to afford tert-butyl (R,E)-3-(4-(3H-[1,2,3]triazolo[4,5-b]pyridin-3-yl)-N-(7-(3-amino-3-oxoprop-1-en-1-yl)isoquinolin-1-yl)-2-fluorobenzamido)-piperidine-1-carboxylate (30 mg) as an off-white solid. LC-MS: [M+H⁺]=537.2.

### Step 3:

The title compound hydrochloride salt was obtained as an off-white solid by deprotection Method A used in Example 97. LC-MS: [M+H⁺]=537.2. ¹H NMR (CD₃OD, 400 MHZ): δ 8.82-8.81 (m, 1H), 8.58-8.53 (m, 2H), 8.32-8.28 (m, 1H), 8.12-8.09 (m, 1H), 8.02-7.99 (m, 1H), 7.97-7.92 (m, 2H), 7.87-7.83 (m, 2H), 7.61-7.58 (m, 1H), 7.39-7.35 (m, 1H), 6.97-6.90 (m, 1H), 5.41-5.37 (m, 1H), 4.07-4.04 (m, 1H), 3.83-3.73 (m, 1H), 2.99-2.96 (m, 1H), 2.13-1.99 (m, 3H), 1.45-1.35 (m, 1H), 1.04-0.96 (m, 1H).

### Synthesis of (R)-4-(3H-[1,2,3]triazolo[4,5-b]pyridin-3-yl)-2-fluoro-N-(piperidin-3-yl)-N-(7-(pyrimidin-5-yl)isoquinolin-1-yl)benzamide (Example 150)

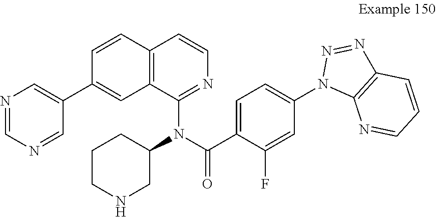 [SMILES: O=C(c1ccc(-n2nnc3cccnc32)cc1F)N(c1nccc2ccc(-c3cncnc3)cc12)[C@@H]1CCCNC1]

Example 150 was prepared according to the procedure described for Example 152 by substituting (1H-pyrazol-5-yl)boronic acid with pyrimidine-5-boronic acid, providing the title compound hydrochloride salt (97%) as an off-white solid. LC-MS: [M+H⁺]=546.8. ¹H NMR (CD₃OD, 400 MHZ): δ 9.32-8.22 (m, 3H), 8.74-8.73 (m, 1H), 8.57-8.41 (m, 3H), 8.09-8.02 (m, 3H), 7.94-7.85 (m, 2H), 7.53-7.50 (m, 1H), 7.29-7.25 (m, 1H), 5.41-5.31 (m, 1H), 4.02-3.99 (m, 1H), 3.77-3.72 (m, 1H), 2.91-2.89 (m, 1H), 2.06-2.03 (m, 1H), 1.96-1.90 (m, 2H), 1.38-1.28 (m, 1H), 0.96-0.89 (m, 1H).

### Synthesis of (R)-4-(3H-[1,2,3]triazolo[4,5-b]pyridin-3-yl)-2-fluoro-N-(piperidin-3-yl)-N-(7-(pyridin-3-yl)isoquinolin-1-yl)benzamide (Example 151)

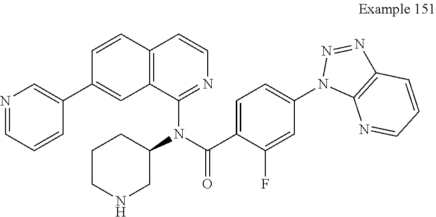 [SMILES: O=C(c1ccc(-n2nnc3cccnc32)cc1F)N(c1nccc2ccc(-c3cccnc3)cc12)[C@@H]1CCCNC1]

Example 151 was prepared according to the procedure described for Example 152 by substituting (1H-pyrazol-5-yl)boronic acid with pyridin-3-ylboronic acid, providing the title compound hydrochloride salt as an off-white solid. LC-MS: [M+H⁺]=545.21. ¹H NMR (CD₃OD, 400 MHZ): δ 9.44 (s, 1H), 9.08-9.07 (m, 2H), 8.91-8.90 (m, 1H), 8.77-8.73 (m, 2H), 8.68-8.63 (m, 1H), 8.34-8.18 (m, 4H), 8.10-8.04 (m, 2H), 7.71-7.68 (m, 1H), 7.47-7.43 (m, 1H), 5.58-5.51 (m, 1H), 4.20-4.17 (m, 1H), 3.96-3.90 (m, 1H), 3.65-3.51 (m, 1H), 3.09-3.07 (m, 1H), 2.25-2.22 (m, 1H), 2.13-2.08 (m, 2H), 1.51-1.41 (m, 1H).

### Synthesis of (R)—N-(7-(1H-pyrazol-5-yl)isoquinolin-1-yl)-4-(3H-[1,2,3]triazolo[4,5-b]pyridin-3-yl)-2-fluoro-N-(piperidin-3-yl)benzamide (Example 152)

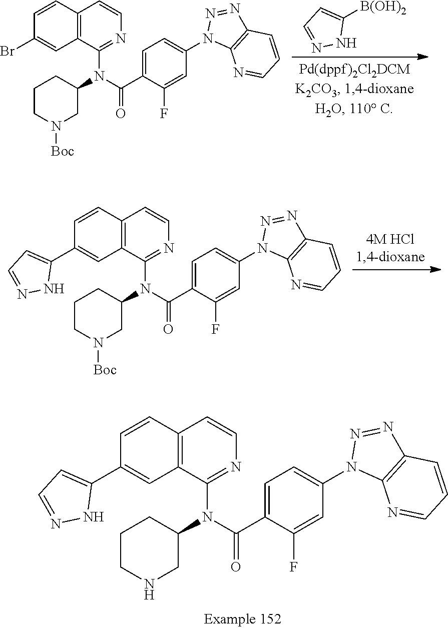

### Step 1:

To a solution of tert-butyl (R)-3-(4-(3H-[1,2,3]triazolo[4,5-b]pyridin-3-yl)-N-(7-bromoisoquinolin-1-yl)-2-fluorobenzamido)piperidine-1-carboxylate (100 mg, 0.154 mmol) in 1,4-dioxane (3 mL) and water (1 mL) at room temperature were added (1H-pyrazol-5-yl)boronic acid (20 mg, 0.178 mmol) and K₂CO₃ The reaction mixture was degassed with nitrogen for 5 min. Then, Pd(dppf)Cl₂·DCM (5.6 mg, 0.007 mmol) was added to reaction mixture. The reaction mixture was heated to 90° C. and stirred for 16 h, after which it was diluted with water (20 mL) and extracted with EtOAc (2×20 mL). The combined organic layer was washed with brine, dried over Na₂SO₄, and concentrated under reduced pressure. The residue was purified by Combi-flash using 80% EtOAc in hexane to afford tert-butyl (R)-3-(N-(7-(1H-pyrazol-5-yl)isoquinolin-1-yl)-4-(3H-[1,2,3]triazolo[4,5-b]pyridin-3-yl)-2-fluorobenzamido)piperidine-1-carboxylate 3 (30 mg, 0.045 mmol, 30%) as an off-white solid. LC-MS [M+H⁺]=634.85.

### Step 2:

The title compound hydrochloride salt was obtained as an off-white solid by deprotection Method A used in Example 97. LC-MS: [M+H⁺]=534.85. ¹H NMR (CD₃OD, 400 MHZ): δ 8.82-8.81 (m, 1H), 8.64 (bs, 1H), 8.64-8.51 (m, 2H), 8.31-8.25 (m, 1H), 8.11-8.08 (m, 1H), 7.99-7.97 (m, 2H), 7.94-7.92 (m, 1H), 7.87-7.86 (m, 1H), 7.62-7.60 (m, 1H), 7.42-7.38 (m, 1H), 7.05-7.04 (m, 1H), 5.46-5.43 (m, 1H), 4.11-4.07 (m, 1H), 3.85-3.80 (m, 1H), 3.01-2.96 (m, 1H), 2.20-2.17 (m, 1H), 2.04-2.00 (m, 2H), 1.48-1.38 (m, 1H), 1.06-0.99 (m, 1H).

### Synthesis of (R)-(1-(4-(3H-[1,2,3]triazolo[4,5-b]pyridin-3-yl)-2-fluoro-N-(piperidin-3-yl)benzamido)isoquinolin-7-yl)methyl methylcarbamate (Example 153)

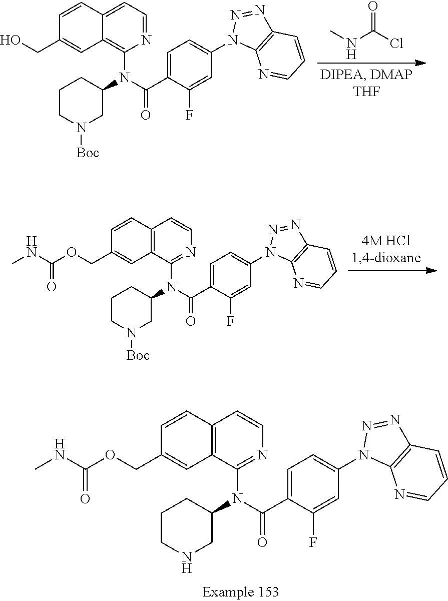

### Step 1:

To a solution of tert-butyl (R)-3-(4-(3H-[1,2,3]triazolo[4,5-b]pyridin-3-yl)-2-fluoro-N-(7-(hydroxymethyl)isoquinolin-1-yl)benzamido)piperidine-1-carboxylate (180 mg, 0.319 mmol) in THF (10 mL) at 0° C. were added DIPEA (0.17 mL, 0.959 mmol) and DMAP (40 mg, 0.319 mmol), followed by methyl carbamic chloride (59 mg, 0.639 mmol). The reaction mixture was warmed to room temperature and stirred for 48 h, after which it was diluted with water (15 mL) and extracted with ethyl acetate (2×30 mL). The combined organic layer was washed with brine, dried over Na₂SO₄, and concentrated under reduced pressure. The residue was purified by Combi-flash using 80% EtOAc in hexane to give tert-butyl (R)-3-(4-(3H-[1,2,3]triazolo[4,5-b]pyridin-3-yl)-2-fluoro-N-(7-(((methylcarbamoyl)oxy)methyl)isoquinolin-1-yl)benzamido)piperidine-1-carboxylate (95 mg, 0.14 mmol, 50%) as an off-white solid. LC-MS: [M+H⁺]=655.25.

### Step 2:

The title compound hydrochloride salt was obtained as an off-white solid by deprotection Method A used in Example 97. LC-MS: [M+H⁺]=555.30. ¹H NMR (CD₃OD, 400 MHZ): δ 8.73 (d, J=3.6 Hz, 1H), 8.49-8.45 (m, 2H), 8.12 (s, 1H), 8.01 (d, J=10.8 Hz, 1H), 7.89-7.82 (m, 2H), 7.77-7.68 (m, 2H), 7.52-7.49 (m, 1H), 7.34-7.30 (m, 1H), 5.33-5.30 (m, 3H), 3.98-3.95 (m, 1H), 3.73-3.67 (m, 1H), 2.85-2.78 (m, 2H), 2.76 (s, 3H), 2.04-2.01 (m, 1H), 2.00-1.91 (m, 2H), 1.38-1.21 (m, 1H).

### Synthesis of (R)-4-(3H-[1,2,3]triazolo[4,5-b]pyridin-3-yl)-2-fluoro-N-(7-(hydroxymethyl)isoquinolin-1-yl)-N-(piperidin-3-yl)benzamide (Example 154)

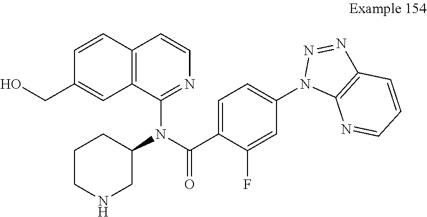 [SMILES: O=C(c1ccc(-n2nnc3cccnc32)cc1F)N(c1nccc2ccc(CO)cc12)[C@@H]1CCCNC1]

Example 154 was prepared according to the procedure described for Example 146 by substituting methyl (R)-1-(4-(3H-[1,2,3]triazolo[4,5-b]pyridin-3-yl)-N-(1-(tertbutoxycarbonyl)piperidin-3-yl)-2-fluorobenzamido)isoquinoline-6-carboxylate with methyl (R)-1-(4-(3H-[1,2,3]triazolo[4,5-b]pyridin-3-yl)-N-(1-(tertbutoxycarbonyl)piperidin-3-yl)-2-fluorobenzamido)isoquinoline-7-carboxylate, providing the title compound hydrochloride salt as an off-white solid. LC-MS: [M+H⁺]=498.20. ¹H NMR (CD₃OD, 400 MHz): δ 8.78-8.72 (m, 1H), 8.49-8.39 (m, 2H), 8.16-8.12 (m, 1H), 8.03-8.00 (m, 1H), 7.92-7.80 (m, 2H), 7.75-7.67 (m, 2H), 7.53-7.51 (m, 1H), 7.39-7.29 (m, 1H), 5.30-5.24 (m, 1H), 3.98-3.95 (m, 1H), 3.96 (d, J=11.6 Hz, 2H), 3.73-3.67 (m, 1H), 2.92-2.82 (m, 1H), 2.52-2.43 (m, 1H), 2.04-2.01 (m, 1H), 1.89-1.82 (m, 2H), 1.82-1.62 (m, 1H), 1.30-1.26 (m, 1H).

### Synthesis of (R)-1-(4-(3H-[1,2,3]triazolo[4,5-b]pyridin-3-yl)-2-fluoro-N-(piperidin-3-yl)benzamido)isoquinoline-7-carboxamide (Example 155)

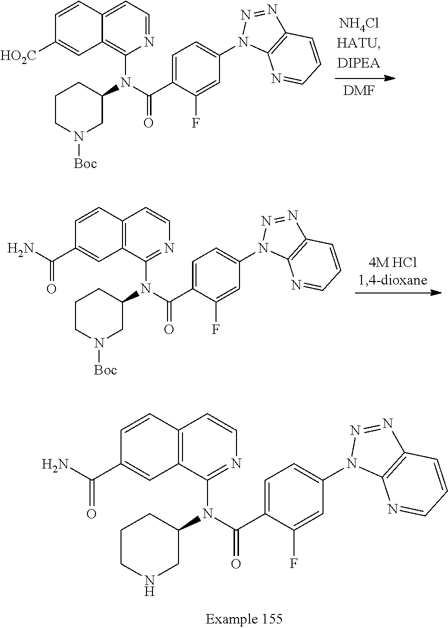

### Step 1:

To a solution of (R)-1-(4-(3H-[1,2,3]triazolo[4,5-b]pyridin-3-yl)-N-(1-(tert-butoxycarbonyl)piperidin-3-yl)-2-fluorobenzamido)isoquinoline-7-carboxylic acid (100 mg, 0.16 mmol) in DMF (8 mL) at room temperature were added DIPEA (0.1 mL, 0.49 mmol), HATU (93 mg, 0.24 mmol) and NH₄Cl (47 mg, 0.49 mmol). The reaction mixture was stirred for 2 h, after which it was diluted with water (30 mL) and extracted with ethyl acetate (2×30 mL). The combined organic layer was washed with brine, dried over Na₂SO₄, and concentrated under reduced pressure. The residue was purified by Combi-flash using 100% EtOAc. The residue was further purified by Prep-HPLC (Conditions: Column: Luna OMEGA C-18 (250×21.2×5 μm), Mobile phase A: 0.1% formic acid in water (100%), Mobile phase B: MeCN (100%), Gradient: T/% B: 0/55, 10/85, Flow: 15 mL/min, Dilution: MeCN+H₂O+THF). Pure fractions were dried by lyophilization to afford tert-butyl (R)-3-(4-(3H-[1,2,3]triazolo[4,5-b]pyridin-3-yl)-N-(7-carbamoylisoquinolin-1-yl)-2-fluorobenzamido)piperidine-1-carboxylate (25 mg, 0.04 mmol, 27%) as an off-white solid. LC-MS: [M+H⁺-Boc]=511.2.

### Step 2:

The title compound hydrochloride salt was obtained as an off-white solid by deprotection Method A used in Example 97. LC-MS: [M+H⁺]=511.2. ¹H NMR (CD₃OD, 400 MHZ): δ 8.91-8.89 (m, 2H), 8.73-8.65 (m, 2H), 8.32-8.38 (m, 1H), 8.19-8.16 (m, 1H), 8.08-8.06 (m, 2H), 8.00-7.99 (m, 1H), 7.71-7.67 (m, 1H), 7.47-7.44 (m, 1H), 5.50-5.47 (m, 1H), 4.18-4.14 (m, 1H), 3.96-3.90 (m, 1H), 3.09-3.05 (m, 1H), 2.24-2.20 (m, 1H), 2.10-2.08 (m, 2H), 1.49-1.45 (m, 2H).

### Synthesis of ((R)-4-(3H-[1,2,3]triazolo[4,5-b]pyridin-3-yl)-N-(7-cyanoisoquinolin-1-yl)-2-fluoro-N-(piperidin-3-yl)benzamide (Example 156)

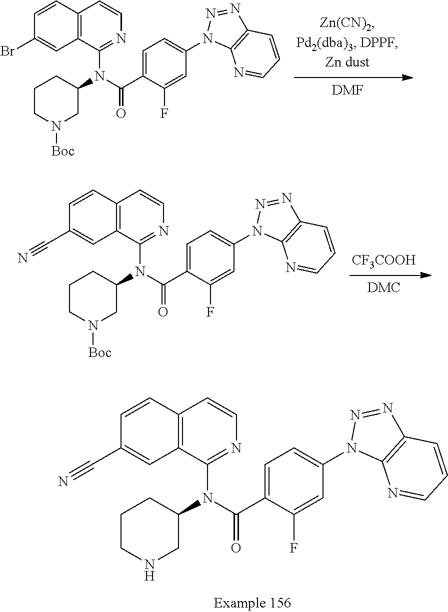

### Step 1:

To a solution of tert-butyl (R)-3-(4-(3H-[1,2,3]triazolo[4,5-b]pyridin-3-yl)-N-(7-bromoisoquinolin-1-yl)-2-fluorobenzamido)piperidine-1-carboxylate (100 mg, 0.15 mmol) in DMF (4 mL) at room temperature were added Zn(CN)₂ (36 mg, 0.30 mmol), DPPF (3 mg, 0.006 mmol) and Zn dust (2 mg, 0.03 mmol). The reaction mixture was degassed with nitrogen for 15 min and followed by addition of Pd₂(dba)₃ (5 mg, 0.006 mmol). The reaction mixture was stirred at 120° C. for 3 h, after which it was diluted with water (15 mL) and extracted with ethyl acetate (2×30 mL). The combined organic layer was washed with brine, dried over Na₂SO₄, and concentrated under reduced pressure. The residue was purified by Combi-flash using 45% EtOAc in hexane. The compound was further purified by Prep-HPLC (Conditions: Column: Luna OMEGA C-18 (250×21.2×5 μm), Mobile phase A: 0.1% formic acid in H₂O (100%), Mobile phase B: 100% MeCN, Gradient: T/% B: 0/60, 10/85, Flow: 15 mL/min, Dilution: MeCN+H₂O+THF). Pure fractions were dried by lyophilization to afford tert-butyl (R)-3-(4-(3H-[1,2,3]triazolo[4,5-b]pyridin-3-yl)-N-(7-cyanoisoquinolin-1-yl)-2 fluorobenzamido)piperidine-1-carboxylate (55 mg) as an off-white solid. LC-MS=[H+H⁺]=493.2.

### Step 2:

To a solution of tert-butyl (R)-3-(4-(3H-[1,2,3]triazolo[4,5-b]pyridin-3-yl)-N-(7-cyanoisoquinolin-1-yl)-2-fluorobenzamido)piperidine-1-carboxylate 6 (50 mg, 0.08 mmol) in DCM (3 mL) at 0° C. was added TFA (1 mL). The reaction was stirred at room temperature for 2 h, after which is was concentrated under reduced pressure and dried by lyophilization to afford ((R)-4-(3H-[1,2,3]triazolo[4,5-b]pyridin-3-yl)-N-(7-cyanoisoquinolin-1-yl)-2-fluoro-N-(piperidin-3-yl)benzamide trifluoroacetic acid salt (48 mg, 94%) as an off-white solid. LC-MS: [M+H⁺]=493.2. ¹H NMR (CD₃OD, 400 MHZ): δ 8.76-8.73 (m, 2H), 8.64-8.62 (m, 1H), 8.52-8.50 (m, 1H), 8.08-7.98 (m, 2H), 7.89-7.86 (m, 3H), 7.55-7.50 (m, 1H), 7.31-7.27 (m, 1H), 5.32-5.22 (m, 1H), 4.00-3.97 (m, 1H), 2.90-2.87 (m, 1H), 2.04-1.93 (m, 3H), 1.30-1.22 (m, 3H).

### Synthesis of (R)-4-(3H-[1,2,3]triazolo[4,5-b]pyridin-3-yl)-2-fluoro-N-(piperidin-3-yl)-N-(3-(thieno[3,2-c]pyridin-2-yl)pyridin-2-yl)benzamide (Example 157)

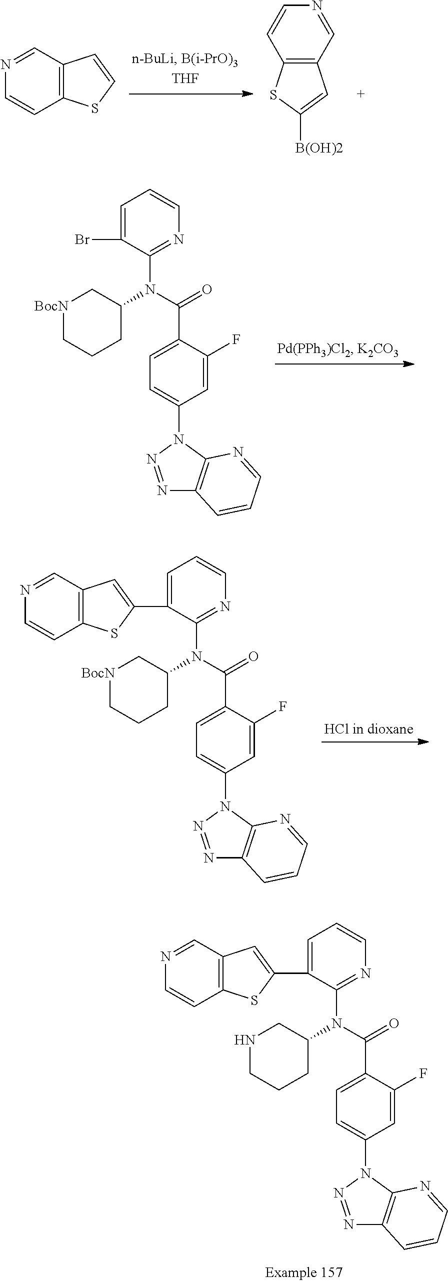

### Step 1:

To a solution of thieno[3,2-c]pyridine (1 g, 7.40 mmol) in THF (5 mL) was dropwise added n-BuLi (2.5 M, 3.55 mL) at −78° C. under N₂ atmosphere. The mixture was stirred at −78° C. for 1 h and followed by addition of triisopropyl borate (2.04 mL, 8.88 mmol) was added. Then the mixture was slowly warmed up to 20° C. and stirred at 20° C. for 1 h. The reaction mixture was quenched by aqueous NH₄Cl solution (30 mL) at 0° C., and then partitioned between water (20 mL) and EtOAc (3×50 mL). The combined organic layer was washed with brine, dried over Na₂SO₄, filtered, and concentrated. The residue was purified by prep-HPLC (column: Phenomenex Luna C18 (250×70 mm×15 μm); mobile phase: [H₂O (0.04% HCl)-MeCN]; gradient: 1%-30% B over 20.0 min) and lyophilized to afford thieno[3,2-c]pyridin-2-ylboronic acid (600 mg, 33%) as a white solid.

### Step 2:

A mixture of thieno[3,2-c]pyridin-2-ylboronic acid (67 mg, 335 μmol), tert-butyl (3R)-3-[(3-bromo-2-pyridyl)-[2-fluoro-4-(triazolo[4,5-b]pyridin-3-yl)benzoyl]amino]piperidine-1-carboxylate (111.11 mg, 168 μmol), K₂CO₃ (51 mg, 369 μmol), Pd(dppf)Cl₂ (12.27 mg, 17 μmol) in dioxane (2 mL) and water (0.2 mL) at 20° C. was degassed with N₂; The mixture was then stirred at 100° C. for 12 h under N₂ atmosphere before it was allowed to cool, diluted with water (10 mL) and extracted with EtOAc (3×10 mL). The combined organic layer was washed with brine (30 mL), dried over Na₂SO₄, filtered and concentrated. The residue was purified by prep-HPLC (column: Phenomenex Luna C18 80×30 mm×3 μm; mobile phase: [H₂O (0.04% HCl)-MeCN]; gradient: 30%-60% B over 8.0 min) and lyophilized to afford tert-butyl 3-[[2-fluoro-4-(triazolo[4,5-b]pyridin-3-yl)benzoyl]-(3-thieno[3,2-c]pyridin-2-yl-2-pyridyl)amino]piperidine-1-carboxylate (60 mg, 24%) as a white solid. LC-MS: [M+H⁺]=651.4.

### Step 3:

The title compound hydrochloride salt was obtained as a white solid by deprotection Method A used in Example 97. LC-MS: [M+H⁺]=551.3. ¹H NMR (400 MHZ, DMSO-d₆): δ 9.09-9.44 (m, 3H), 8.85-8.96 (m, 1H), 8.72-8.81 (m, 2H), 8.62-8.70 (m, 1H), 8.32-8.60 (m, 1H), 8.11-8.18 (m, 1H), 7.91-8.02 (m, 1H), 7.76-7.90 (m, 2H), 7.66 (dd, J=8.32, 4.57 Hz, 1H), 7.53-7.62 (m, 1H), 6.78-6.96 (m, 1H), 5.03-5.17 (m, 1H), 3.74 (br s, 2H), 3.29 (br s, 1H), 2.81-2.91 (m, 1H), 1.85-2.02 (m, 3H), 1.38-1.55 (m, 1H).

### Synthesis of (R,E)-4-(3H-[1,2,3]triazolo[4,5-b]pyridin-3-yl)-2-fluoro-N-(2-(3-hydroxyprop-1-en-1-yl)thieno[3,2-c]pyridin-4-yl)-N-(piperidin-3-yl)benzamide (Example 158)

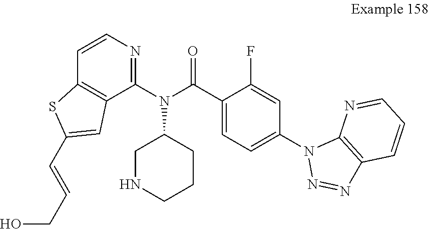 [SMILES: O=C(c1ccc(-n2nnc3cccnc32)cc1F)N(c1nccc2sc(/C=C/CO)cc12)[C@@H]1CCCNC1]

Example 158 was prepared according to the procedure described for Example 161 by substituting 4-pyridylboronic acid with (E)-tert-butyldimethyl((3-(4,4,5,5-tetramethyl-1,3,2-dioxaborolan-2-yl)allyl)oxy)silane, provided the title compound hydrochloride acid salt as an off-white solid. LC-MS: [M+H⁺]=530.3. ¹H NMR (400 MHZ, CD₃OD): δ 8.81-8.72 (m, 1H), 8.56-8.47 (m, 1H), 8.34-8.23 (m, 1H), 8.13-7.93 (m, 2H), 7.74 (br d, J=4.3 Hz, 1H), 7.58-7.49 (m, 1H), 7.44-7.32 (m, 2H), 7.01-6.89 (m, 1H), 6.45-6.27 (m, 1H), 4.58 (br s, 1H), 4.26 (br d, J=3.9 Hz, 2H), 3.66-3.55 (m, 1H), 3.42-3.33 (m, 2H), 3.05-2.93 (m, 1H), 2.05-1.85 (m, 1H), 1.78-1.61 (m, 2H), 1.39-1.16 (m, 1H). ¹⁹F NMR (377 MHz, DMSO-d₆): δ-73.41 (s, 1F), −109.04-−113.30 (m, 1F).

### Synthesis of (R)-4-(3H-[1,2,3]triazolo[4,5-b]pyridin-3-yl)-2-fluoro-N-(piperidin-3-yl)-N-(2-(1,2,3,6-tetrahydropyridin-4-yl)thieno[3,2-c]pyridin-4-yl)benzamide (Example 159)

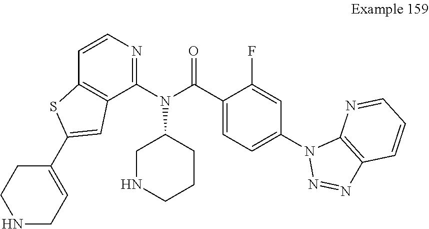 [SMILES: O=C(c1ccc(-n2nnc3cccnc32)cc1F)N(c1nccc2sc(C3=CCNCC3)cc12)[C@@H]1CCCNC1]

Example 159 was prepared according to the procedure described for Example 161 by substituting 4-pyridylboronic acid with tert-butyl 4-(4,4,5,5-tetramethyl-1,3,2-dioxaborolan-2-yl)-3,6-dihydropyridine-1(2H)-carboxylate, provided the title compound hydrochloride acid salt as an off-white solid. LC-MS: [M+H⁺]=555.3. ¹H NMR (400 MHZ, CD₃OD): δ 8.78 (br d, J=4.00 Hz, 1H), 8.54 (br d, J=8.13 Hz, 1H), 8.40 (d, J=5.50 Hz, 1H), 7.98-8.16 (m, 2H), 7.84 (br d, J=5.25 Hz, 1H), 7.34-7.52 (m, 2H), 6.34 (br s, 1H), 5.12-5.42 (m, 1H), 3.90 (br s, 3H), 3.46-3.75 (m, 3H), 3.35-3.41 (m, 1H), 2.81-3.09 (m, 3H), 1.86-2.10 (m, 3H), 1.25-1.47 (m, 1H). ¹⁹F NMR (376 MHz, DMSO-d₆): δ −108.61-114.58 (m, 1F).

### Synthesis of (R)—N-(2-(1H-pyrazol-4-yl)thieno[3,2-c]pyridin-4-yl)-4-(3H-[1,2,3]triazolo[4,5-b]pyridin-3-yl)-2-fluoro-N-(piperidin-3-yl)benzamide (Example 160)

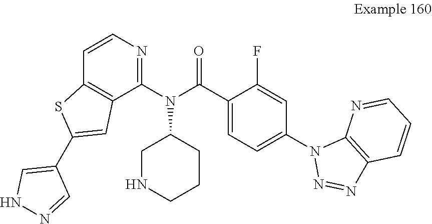 [SMILES: O=C(c1ccc(-n2nnc3cccnc32)cc1F)N(c1nccc2sc(-c3cn[nH]c3)cc12)[C@@H]1CCCNC1]

Example 160 was prepared according to the procedure described for Example 161 by substituting 4-pyridylboronic acid with 4-(4,4,5,5-tetramethyl-1,3,2-dioxaborolan-2-yl)-1-((2-(trimethylsilyl) ethoxy)methyl)-1H-pyrazole, provided the title compound hydrochloride acid salt as an off-white solid. LC-MS: [M+H⁺]=540.3. ¹H NMR (400 MHZ, CD₃OD): δ 8.75 (br d, J=3.8 Hz, 1H), 8.51 (br d, J=8.5 Hz, 1H), 8.33 (br d, J=5.5 Hz, 1H), 8.17-8.07 (m, 3H), 8.05-7.97 (m, 1H), 7.84 (br d, J=5.3 Hz, 1H), 7.61 (s, 1H), 7.57-7.49 (m, 1H), 7.43 (br t, J=8.3 Hz, 1H), 5.33-5.15 (m, 1H), 3.98-3.83 (m, 1H), 3.73-3.59 (m, 1H), 3.39-3.34 (m, 1H), 2.98-2.81 (m, 1H), 2.19-2.05 (m, 1H), 2.01-1.81 (m, 2H), 1.50-1.33 (m, 1H). 19F NMR (377 MHz, DMSO-d₆): δ −107.98-−113.09 (m, 1F).

### Synthesis of (R)-4-(3H-[1,2,3]triazolo[4,5-b]pyridin-3-yl)-2-fluoro-N-(piperidin-3-yl)-N-(2-(pyridin-4-yl)thieno[3,2-c]pyridin-4-yl)benzamide (Example 161)

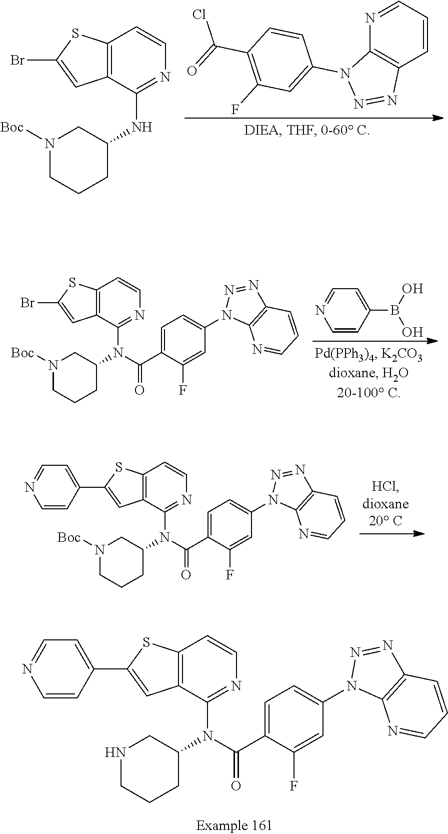

### Step 1:

To a solution of tert-butyl (3R)-3-[(2-bromothieno[3,2-c]pyridin-4-yl)amino]piperidine-1-carboxylate (10 g, 24.25 mmol) in THF (100 mL) was added DIEA (21.12 mL, 121.26 mmol) and 2-fluoro-4-(triazolo[4,5-b]pyridin-3-yl)benzoyl chloride (13.42 g, 48.50 mmol) at 20° C. under N₂ atmosphere. Then the mixture was stirred at 60° C. for 2 h before it was filtered. The filtrate was diluted with water (200 mL) and extracted with EtOAc (3×200 mL). The combined organic layer was washed with brine (100 mL), dried over Na₂SO₄, filtered and concentrated under reduced pressure to give crude tert-butyl (R)-3-(4-(3H-[1,2,3]triazolo[4,5-b]pyridin-3-yl)-N-(2-bromothieno[3,2-c]pyridin-4-yl)-2-fluorobenzamido)piperidine-1-carboxylate (15 g, 85%) as a brown solid.

### Step 2:

To a solution of tert-butyl (3R)-3-[(2-bromothieno[3,2-c]pyridin-4-yl)-[2-fluoro-4-(triazolo[4,5-b]pyridin-3-yl)benzoyl]amino]piperidine-1-carboxylate (8 g, 11.03 mmol) in dioxane (72 mL) and H₂O (8 mL) at 20° C. under N₂ atmosphere was added 4-pyridylboronic acid (4.07 g, 33.10 mmol), K₂CO₃ (4.57 g, 33.10 mmol) and Pd(dppf)Cl₂ (807-mg, 1.10 mmol). The reaction mixture was purged with N₂ thrice and then stirred at 100° C. for 1 h. The reaction mixture was filtered, and the filtrate diluted with water (100 mL) and extracted with EtOAc (3×100 mL). The combined organic layer was washed with brine (50 mL), dried over Na₂SO₄, filtered and concentrated. The residue was purified by flash column chromatography (SiO₂, Petroleum ether/Ethyl acetate=3/2 to 1/1) to give tert-butyl(3R)-3-[2-fluoro-4-(triazolo[4,5-b]pyridin-3-yl)benzoyl]-[2-(4-pyridyl)thieno[3,2-c]pyridin-4-yl]amino]piperidine-1-carboxylate (7.5 g, 75%) as a yellow solid.

### Step 3:

The title compound hydrochloride salt was obtained as an off-white solid by deprotection Method A used in Example 97. LC-MS: [M+H]=551.4. ¹H NMR (400 MHZ, DMSO-d₆): δ 9.52-9.11 (m, 2H), 8.89-8.76 (m, 3H), 8.70-8.57 (m, 2H), 8.46 (br d, J=5.5 Hz, 1H), 8.14 (br t, J=5.7 Hz, 3H), 7.98 (br d, J=10.6 Hz, 1H), 7.88 (br d, J=8.4 Hz, 1H), 7.62-7.51 (m, 2H), 5.25-4.98 (m, 1H), 3.73 (br s, 1H), 3.39-3.32 (m, 1H), 3.19 (br d, J=11.1 Hz, 1H), 2.81-2.63 (m, 1H), 2.32-1.74 (m, 3H), 1.28-1.05 (m, 1H).

### Synthesis of (R)-4-(3H-[1,2,3]triazolo[4,5-b]pyridin-3-yl)-N-(2-bromothieno[3,2-c]pyridin-4-yl)-2-fluoro-N-(piperidin-3-yl)benzamide (Example 162)

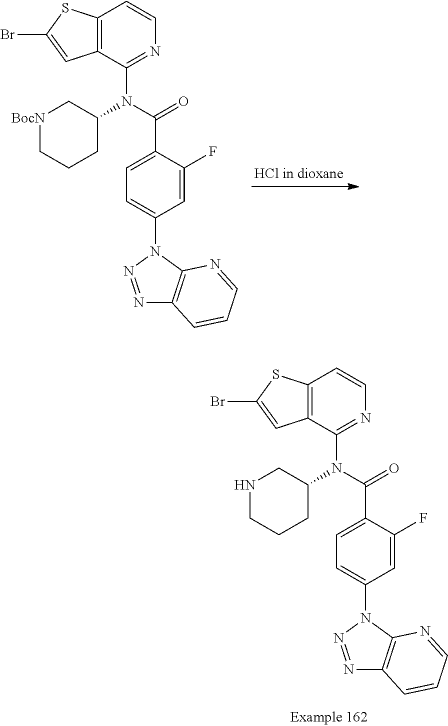 [SMILES: CC(C)(C)OC(=O)N1CCC[C@@H](N(C(=O)c2ccc(-n3nnc4cccnc43)cc2F)c2nccc3sc(Br)cc23)C1 ; O=C(c1ccc(-n2nnc3cccnc32)cc1F)N(c1nccc2sc(Br)cc12)[C@@H]1CCCNC1]

Example 162 was prepared according to the procedure described for Example 161 by deprotecting the Boc group with 4M HCl in dioxane, provided the title compound hydrochloride acid salt as an off-white solid. LC-MS: [M+H⁺]=554.0. ¹H NMR (400 MHZ, DMSO-d₆): δ 9.08-8.93 (m, 1H), 8.89-8.77 (m, 2H), 8.74-8.65 (m, 1H), 8.44-8.32 (m, 1H), 8.08-7.98 (m, 2H), 7.96-7.86 (m, 2H), 7.66-7.58 (m, 1H), 7.52-7.41 (m, 1H), 5.12-4.81 (m, 1H), 3.80-3.62 (m, 1H), 3.24-3.08 (m, 2H), 2.87-2.69 (m, 1H), 2.11-1.66 (m, 4H), 1.27-1.09 (m, 1H).

### Synthesis of (R,E)-4-(3H-[1,2,3]triazolo[4,5-b]pyridin-3-yl)-2-fluoro-N-(piperidin-3-yl)-N-(8-(2-(pyridin-3-yl)vinyl)isoquinolin-1-yl)benzamide (Example 163)

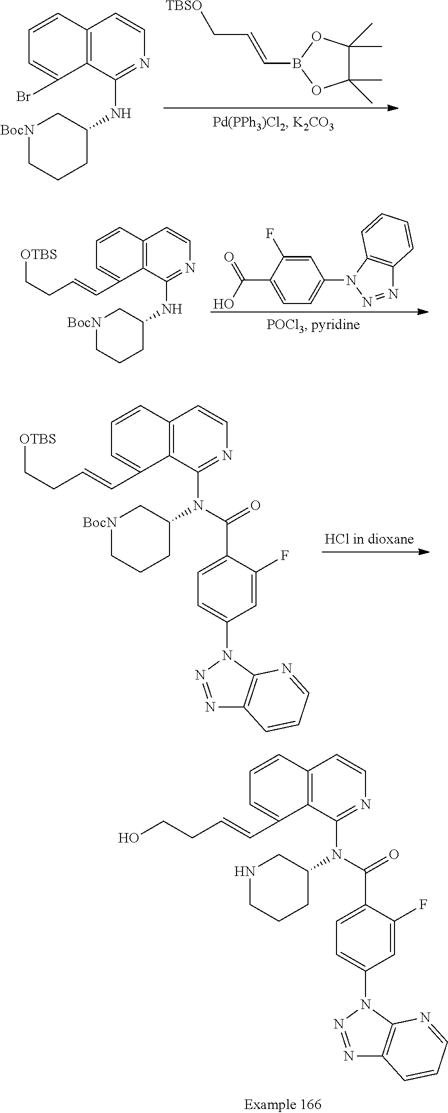

### Step 1:

To a solution of tert-butyl (R)-3-((8-bromoisoquinolin-1-yl)amino)piperidine-1-carboxylate (0.3 g, 738 μmol) in dioxane (5 mL) and H₂O (1 mL) was added (E)-3-(2-(4,4,5,5-tetramethyl-1,3,2-dioxaborolan-2-yl)vinyl)pyridine (205 mg, 886 μmol), Cs₂CO₃ (722 mg, 2.22 mmol) and Pd(dppf)Cl₂ (54 mg, 74 μmol) at 20° C. under N₂atmosphere. The reaction mixture was stirred at 80° C. for 2 h. The reaction mixture was filtered and concentrated under reduced pressure. The residue was purified by flash column chromatography (SiO₂, petroleum ether/ethyl acetate=5/1 to 3/1) to give tert-butyl (R,E)-3-((8-(2-(pyridin-3-yl)vinyl)isoquinolin-1-yl)amino)piperidine-1-carboxylate (300 mg, 76%) as a yellow oil. LC-MS: [M+H⁺]=431.3.

### Step 2:

To a solution of tert-butyl (R,E)-3-((8-(2-(pyridin-3-yl)vinyl)isoquinolin-1-yl)amino)piperidine-1-carboxylate (190 mg, 441.31 μmol) in pyridine (3 mL) was added 2-fluoro-4-(triazolo[4,5-b]pyridin-3-yl)benzoic acid (170.92 mg, 661.96 μmol) and POCl₃ (205.67 μL, 2.21 mmol) at 0° C. under N₂ atmosphere. The mixture was stirred at 50° C. for 12 h before it was allowed to cool. The reaction mixture was poured into ice-cold H₂O (10 mL) very slowly over 10 min. The aqueous phase was extracted with ethyl acetate (3×5 mL). The combined organic phase was washed with brine (15 mL), dried over anhydrous Na₂SO₄, filtered, and concentrated. The residue was purified by prep-HPLC (column: Waters Xbridge BEH C18 100×30 mm×10 μm; mobile phase: [A: H₂O (10 mM NH₄HCO₃); B: MeCN]; B %: 40.00%-70.00%, 8.00 min) to give tert-butyl (R,E)-3-(4-(3H-[1,2,3]triazolo[4,5-b]pyridin-3-yl)-2-fluoro-N-(8-(2-(pyridin-3-yl)vinyl) isoquinolin-1-yl)benzamido)piperidine-1-carboxylate (50 mg, 16%) as a yellow solid. LC-MS: [M+H⁺]=671.2.

### Step 3:

To a solution of tert-butyl (R,E)-3-(4-(3H-[1,2,3]triazolo[4,5-b]pyridin-3-yl)-2-fluoro-N-(8-(2-(pyridin-3-yl)vinyl) isoquinolin-1-yl)benzamido)piperidine-1-carboxylate (50 mg, 74.55 μmol) in DCM (2 mL) was added TMSOTf (80.82 μL, 447 μmol) and 2,6-dimethylpyridine (52.09 μL, 447 μmol) at 0° C. under N₂ atmosphere. The reaction mixture was stirred at 20° C. for 2 h under N₂ atmosphere it was filtered, and the filtrate was concentrated under reduced pressure. The residue was purified by prep-HPLC (column: Waters Xbridge BEH C18 100×30 mm×10 μm; mobile phase: [A: H₂O (10 mM NH₄HCO₃); B: Me CN]; B %: 30.00%-60.00%, 8.00 min) to give (R,E)-4-(3H-[1,2,3]triazolo[4,5-b]pyridin-3-yl)-2-fluoro-N-(piperidin-3-yl)-N-(8-(2-(pyridin-3-yl)vinyl)isoquinolin-1-yl)benzamide (20.1 mg, 45%) as a white solid. LC-MS: [M+H⁺]=571.2. ¹H NMR (400 MHZ, DMSO-d₆): δ 8.92 (d, J=2.13 Hz, 1H), 8.76-8.84 (m, 1H), 8.67 (dd, J=8.32, 1.19 Hz, 1H), 8.57 (d, J=4.63 Hz, 1H), 8.44-8.52 (m, 1H), 8.12-8.18 (m, 1H), 7.98-8.11 (m, 1H), 7.77-7.96 (m, 5H), 7.68-7.76 (m, 1H), 7.57 (ddd, J=19.51, 8.19, 4.69 Hz, 2H), 7.19-7.30 (m, 2H), 4.44-4.64 (m, 1H), 3.51 (br d, J=11.01 Hz, 1H), 3.09 (t, J=10.82 Hz, 1H), 2.76-2.89 (m, 1H), 2.07-2.36 (m, 2H), 1.65-1.76 (m, 1H), 1.34-1.59 (m, 2H), 1.04 (qd, J=12.24, 4.06 Hz, 1H).

### Synthesis of (R)-4-(3H-[1,2,3]triazolo[4,5-b]pyridin-3-yl)-N-(8-cyanoisoquinolin-1-yl)-2-fluoro-N-(piperidin-3-yl)benzamide (Example 164)

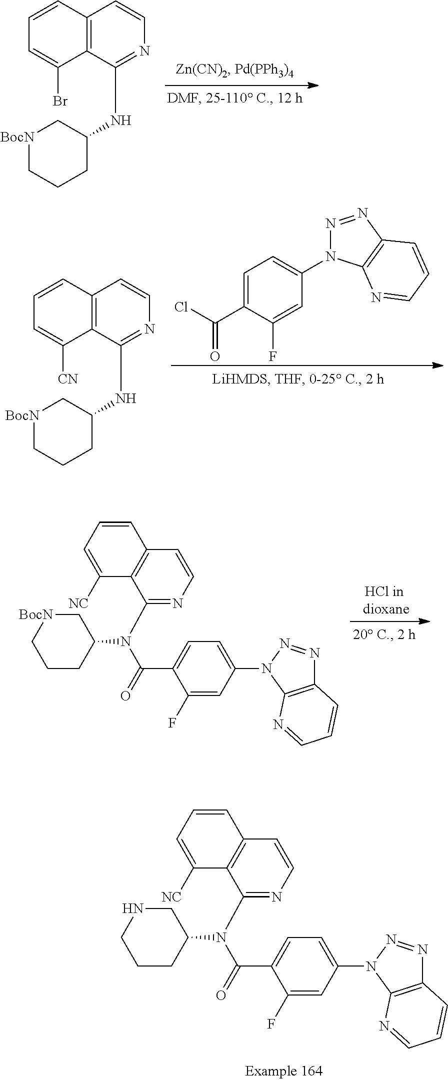

To a solution of tert-butyl (R)-3-((8-bromoisoquinolin-1-yl)amino)piperidine-1-carboxylate (0.3 g, 738.34 μmol) in DMF (5 mL) at 25° C. under N₂ atmosphere was added Zn(CN)₂ (346.80 mg, 2.95 mmol) and Pd(PPh₃) 4 (128 mg, 110.75 μmol). The reaction mixture was stirred at 110° C. for 12 h. The reaction mixture was poured into H₂O (10 mL). The aqueous phase was extracted with ethyl acetate (3×15 mL), the combined organic phase was washed with brine (15 mL), dried over anhydrous Na₂SO₄, filtered, and concentrated. The residue was purified by prep-HPLC (column: Phenomenex Luna C18 75×30 mm×3 μm; mobile phase: [H₂O (0.04% HCl)-MeCN]; gradient: 60%-85% B over 8.0 min) to give tert-butyl (R)-3-((8-cyanoisoquinolin-1-yl)amino)piperidine-1-carboxylate (150 mg, 54%) as a yellow solid.

### Steps 2:

To a solution of 4-(3H-[1,2,3]triazolo[4,5-b]pyridin-3-yl)-2-fluorobenzoic acid (180 mg, 697 μmol) in toluene (0.5 mL) was added SOCl₂ (1 mL) at 20° C. under N₂ atmosphere. The reaction mixture was stirred at 60° C. for 2 h. The reaction mixture was concentrated under reduced pressure to give crude 4-(3H-[1,2,3]triazolo[4,5-b]pyridin-3-yl)-2-fluorobenzoyl chloride (190 mg) which was used in next step directly without purification.

To a solution of tert-butyl (R)-3-((8-cyanoisoquinolin-1-yl)amino)piperidine-1-carboxylate (150 mg, 426 μmol) in THF (8 mL) at 0° C. under N₂ atmosphere was added LiHMDS in THF (1 M, 1.28 mL). The mixture was stirred at 0° C. for 0.5 h and followed by addition of 4-(3H-[1,2,3]triazolo[4,5-b]pyridin-3-yl)-2-fluorobenzoyl chloride (176.62 mg, 638 μmol). The reaction mixture was then stirred at 25° C. for 1.5 h before it was poured into saturated aq. NH₄Cl (10 mL) over 10 min. The aqueous phase was extracted with ethyl acetate (3×8 mL). The combined organic phase was washed with brine (8 mL), dried over anhydrous Na₂SO₄, filtered, and concentrated. The residue was purified by Prep-TLC (SiO₂, petroleum ether/ethyl acetate=1:3) to give tert-butyl (R)-3-(4-(3H-[1,2,3]triazolo[4,5-b]pyridin-3-yl)-N-(8-cyanoisoquinolin-1-yl)-2-fluorobenzamido)piperidine-1-carboxylate (80 mg, 31%) as a yellow solid.

### Step 3:

The title compound hydrochloride salt was obtained as an off-white solid by deprotection Method A used in Example 97. LC-MS: [M+H⁺]=493.3. ¹H NMR (400 MHZ, DMSO-d₆): δ 9.01-9.12 (m, 1H), 8.81 (dd, J=4.50, 1.38 Hz, 1H), 8.66-8.70 (m, 2H), 8.41-8.44 (m, 1H), 8.30 (d, J=7.75 Hz, 1H), 8.06 (d, J=5.63 Hz, 1H), 7.84-7.97 (m, 4H), 7.60 (dd, J=8.32, 4.57 Hz, 1H), 7.35 (t, J=8.00 Hz, 1H), 4.96-5.09 (m, 1H), 3.84-3.93 (m, 1H), 3.46 (br t, J=11.57 Hz, 1H), 3.24 (br d, J=12.38 Hz, 1H), 2.74-2.82 (m, 1H), 1.79-1.89 (m, 3H), 1.23 (s, 1H), 1.02-1.14 (m, 1H).

### Synthesis of (R,E)-4-(3H-[1,2,3]triazolo[4,5-b]pyridin-3-yl)-2-fluoro-N-(8-(3-hydroxyprop-1-en-1-yl)isoquinolin-1-yl)-N-(piperidin-3-yl)benzamide (Example 165)

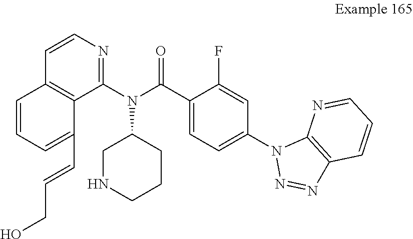 [SMILES: O=C(c1ccc(-n2nnc3cccnc32)cc1F)N(c1nccc2cccc(/C=C/CO)c12)[C@@H]1CCCNC1]

Example 165 was prepared according to the procedure described for Example 163 by substituting (E)-3-(2-(4,4,5,5-tetramethyl-1,3,2-dioxaborolan-2-yl)vinyl)pyridine with tert-butyl-dimethyl-[(E)-3-(4,4,5,5-tetramethyl-1,3,2-dioxaborolan-2-yl)allyloxy]silane, provided the title compound hydrochloride acid salt as an off-white solid. LC-MS: [M+H⁺]=524.2. ¹H NMR (400 MHZ, DMSO-d₆): δ 8.81 (dd, J=1.1, 4.5 Hz, 1H), 8.67 (dd, J=1.3, 8.3 Hz, 1H), 8.45-8.37 (m, 1H), 7.91-7.84 (m, 1H), 7.81-7.72 (m, 3H), 7.69-7.62 (m, 2H), 7.59 (dd, J=4.6, 8.3 Hz, 1H), 7.42-7.16 (m, 2H), 6.33-6.04 (m, 2H), 4.98-4.82 (m, 1H), 4.57-4.33 (m, 1H), 4.28 (br d, J=3.6 Hz, 2H), 3.55 (br d, J=10.8 Hz, 1H), 3.11-3.00 (m, 2H), 2.86-2.75 (m, 1H), 2.22-2.12 (m, 1H), 1.66-1.41 (m, 3H).

### Synthesis of (R,E)-4-(3H-[1,2,3]triazolo[4,5-b]pyridin-3-yl)-2-fluoro-N-(8-(4-hydroxybut-1-en-1-yl)isoquinolin-1-yl)-N-(piperidin-3-yl)benzamide (Example 166)

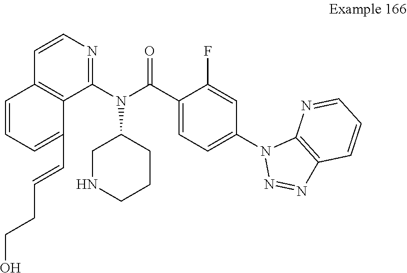 [SMILES: O=C(c1ccc(-n2nnc3cccnc32)cc1F)N(c1nccc2cccc(/C=C/CCO)c12)[C@@H]1CCCNC1]

Example 166 was prepared according to the procedure described for Example 163 by substituting (E)-3-(2-(4,4,5,5-tetramethyl-1,3,2-dioxaborolan-2-yl)vinyl)pyridine with (E)-tert-butyldimethyl((4-(4,4,5,5-tetramethyl-1,3,2-dioxaborolan-2-yl)but-3-en-1-yl)oxy)silane, provided the title compound hydrochloride acid salt as an off-white solid. LC-MS: [M+H⁺]=538.3. ¹H NMR (400 MHZ, DMSO-d₆): δ 0.92-1.14 (m, 1H), 1.41-1.77 (m, 3H), 2.01-2.17 (m, 1H), 2.20-2.38 (m, 2H), 2.61-2.71 (m, 1H), 2.78-2.89 (m, 1H), 3.06 (br t, J=10.76 Hz, 1H), 3.44-3.60 (m, 1H), 3.63-3.79 (m, 2H), 4.35-4.57 (m, 1H), 4.70 (t, J=5.25 Hz, 1H), 6.11-6.29 (m, 1H), 7.11-7.26 (m, 2H), 7.57-7.67 (m, 3H), 7.73-7.81 (m, 3H), 7.88 (dd, J=10.76, 1.88 Hz, 1H), 8.35-8.46 (m, 1H), 8.67 (dd, J=8.38, 1.38 Hz, 1H), 8.82 (dd, J=4.50, 1.38 Hz, 1H).

### Synthesis of methyl (R)-2-(4-(3H-[1,2,3]triazolo[4,5-b]pyridin-3-yl)-2-fluoro-N-(piperidin-3-yl)benzamido)nicotinate (Example 167)

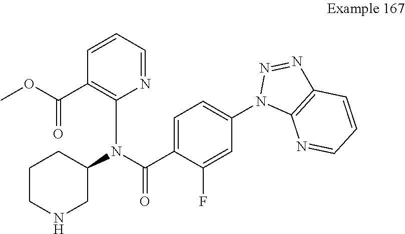 [SMILES: COC(=O)c1cccnc1N(C(=O)c1ccc(-n2nnc3cccnc32)cc1F)[C@@H]1CCCNC1]

To a solution of methyl (R)-2-(4-(3H-[1,2,3]triazolo[4,5-b]pyridin-3-yl)-N-(1-(tert-butoxycarbonyl)piperidin-3-yl)-2-fluorobenzamido)nicotinate (50 mg, 0.086 mmol) in DCM (2 mL) at 0° C. was added 4M HCl in 1,4-dioxane (5 mL). The reaction mixture was stirred at room temperature for 2 h before it was concentrated. The residue was purified by Prep-HPLC (Conditions: Column: Luna OMEGA C-18 (250×21.2×5 μm), Mobile phase A: 0.1% formic acid in water (100%), Mobile phase B: MeCN (100%), Gradient: Time (min)/% B: 0/10, 2/10, 10/95, Flow: 15 mL/min, Dilution: MeCN+H2O+THF). Pure fractions were combined and dried by lyophilization to afford methyl (R)-2-(4-(3H-[1,2,3]triazolo[4,5-b]pyridin-3-yl)-2-fluoro-N-(piperidin-3-yl)benzamido)nicotinate formate salt (25 mg) as an off-white solid. LC-MS: [M+H⁺]=476.2. ¹H NMR (CD₃OD, 400 MHz): δ 8.81-8.80 (m, 1H), 8.75-8.72 (m, 1H), 8.56-8.53 (m, 2H), 8.16-8.1 (m, 3H), 7.59-7.53 (m, 1H), 7.45-7.41 (m, 1H), 7.37-7.31 (m, 1H), 5.03-4.96 (m, 1H), 3.90 (s, 3H), 3.81-3.77 (m, 1H), 3.73-3.66 (m, 1H), 2.88-2.82 (m, 1H), 2.43-2.42 (m, 1H), 2.62-1.84 (m, 3H), 1.62-1.55 (m, 1H).

### Synthesis of (R)-2-fluoro-4-(hydroxymethyl)-N-(8-methylisoquinolin-1-yl)-N-(piperidin-3-yl)benzamide (Example 168)

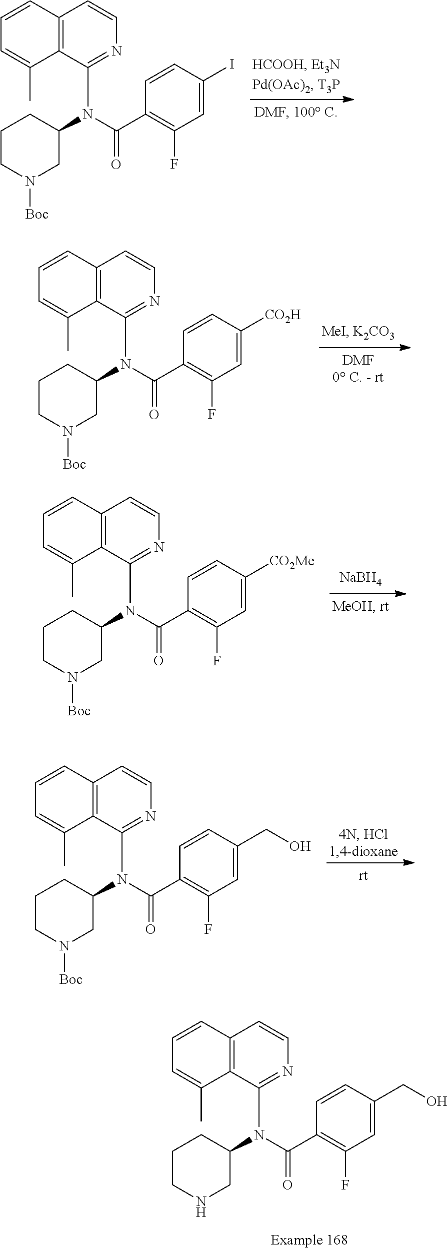

### Step 1:

To a solution of tert-butyl (R)-3-(2-fluoro-4-iodo-N-(8-methylisoquinolin-1-yl)benzamido)piperidine-1-carboxylate (700 mg, 2.05 mmol) in DMF (10 mL) at room temperature was added triethylamine (0.7 mL, 5.1 mmol), polyphosphoric anhydride (50% in DMF, 1 g, 1.64 mmol), formic acid (0.34 mL, 9.22 mmol), and Pd(OAc)₂ (23 mg, 0.102 mmol). The reaction mixture was then stirred at 100° C. for 5 h before it was cooled, diluted with water (10 mL), and extracted with EtOAc (20 mL). The organic layer was dried over Na₂SO₄ and concentrated under reduced pressure to afford (R)-4-((1-(tert-butoxycarbonyl)piperidin-3-yl)(8-methylisoquinolin-1-yl)carbamoyl)-3-fluorobenzoic acid (650 mg). LC-MS: [M+H⁺]=508.8.

### Step 2:

To a solution of (R)-4-((1-(tert-butoxycarbonyl)piperidin-3-yl)(8-methylisoquinolin-1-yl)carbamoyl)-3-fluorobenzoic acid (600 mg, 1.18 mmol) in DMF (5 mL) was added K₂CO₃ (326 mg, 2.36 mmol) at room temperature. Methyl iodide (0.146 mL, 2.36 mmol) was added to reaction mixture at 0° C. and then was stirred at room temperature for 1 h. The reaction mixture was diluted with water (10 mL) and extracted with EtOAc (20 mL). The organic layer was dried over Na₂SO₄, filtered, and concentrated under reduced pressure to afford tert-butyl (R)-3-(2-fluoro-4-(methoxycarbonyl)-N-(8-methylisoquinolin-1-yl)benzamido)piperidine-1-carboxylate (500 mg, 81%). LC-MS: [M+H⁺]=522.8.

### Step 3:

To a solution of tert-butyl (R)-3-(2-fluoro-4-(methoxycarbonyl)-N-(8-methylisoquinolin-1-yl)benzamido)piperidine-1-carboxylate (300 mg, 0.575 mmol) in methanol (5 mL) at 0° C. was added NaBH₄ (80 mg, 2.30 mmol). The reaction mixture was stirred at room temperature for 2 h. 2 h before it was quenched with water and extracted with ethyl acetate (2×20 mL). The combined organic layer was washed with brine (20 mL), dried over Na₂SO₄, filtered, and concentrated under reduced pressure to afford tert-butyl (R)-3-(2-fluoro-4-(hydroxymethyl)-N-(8-methylisoquinolin-1-yl)benzamido)piperidine-1-carboxylate (50 mg) as an off-white solid. LC-MS: [M+H⁺]=494.8.

### Step 4:

The title compound hydrochloride salt was obtained as an off-white solid by deprotection Method A used in Example 97. LC-MS: [M+H⁺]=431.9. ¹H NMR (CD₃OD, 400 MHZ): δ 8.31 (d, J=5.6 Hz, 1H), 7.62 (d, J=5.6 Hz, 1H), 7.56-7.52 (m, 1H), 7.42-7.33 (m, 2H), 6.71-6.65 (m, 2H), 6.54 (d, J=8.0 Hz, 1H), 4.97-4.91 (m, 1H), 4.24 (s, 2H), 3.89-3.86 (m, 1H), 3.71-3.66 (m, 1H), 3.30-3.27 (m, 1H), 2.91-2.88 (m, 1H), 2.72 (s, 3H), 2.61-2.56 (m, 1H), 1.85-1.81 (m, 2H), 1.45-1.38 (m, 1H).

### Synthesis of (R)-4-([1,2,4]triazolo[1,5-a]pyridin-2-ylamino)-2-fluoro-N-(8-methylisoquinolin-1-yl)-N-(piperidin-3-yl)benzamide (Example 169)

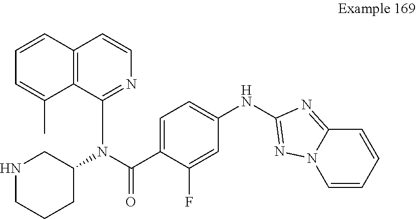 [SMILES: Cc1cccc2ccnc(N(C(=O)c3ccc(Nc4nc5ccccn5n4)cc3F)[C@@H]3CCCNC3)c12]

Example 169 was prepared according to the procedure described for Example 131 by substituting 3-amino-1-methyl-pyridin-2-one with [1,2,4]triazolo[1,5-a]pyridin-2-amine, provided the title compound hydrochloride acid salt as an off-white solid. LC-MS: [M+H⁺]=496.2. ¹H NMR (400 MHZ, DMSO-d₆): δ 9.90 (s, 1H), 9.36-9.58 (m, 1H), 9.01-9.24 (m, 1H), 8.76 (d, J=6.63 Hz, 1H), 8.41-8.50 (m, 1H), 7.78-7.83 (m, 1H), 7.72 (br d, J=8.00 Hz, 1H), 7.50-7.58 (m, 3H), 7.44-7.48 (m, 1H), 7.29-7.38 (m, 1H), 7.03 (dt, J=6.63, 4.19 Hz, 1H), 6.84 (dd, J=8.69, 1.94 Hz, 1H), 6.57-6.68 (m, 1H), 4.93-4.99 (m, 1H), 3.68-3.82 (m, 1H), 3.57-3.62 (m, 1H), 3.53 (br s, 1H), 3.23 (br d, J=12.51 Hz, 1H), 2.70-2.82 (m, 4H), 1.66-1.90 (m, 3H), 1.23 (s, 1H).

### Synthesis of (R)-3-(3-fluoro-4-((8-methylisoquinolin-1-yl)(piperidin-3-yl)carbamoyl)phenyl)-3H-[1,2,3]triazolo[4,5-b]pyridine-5-carboxamide (Example 170)

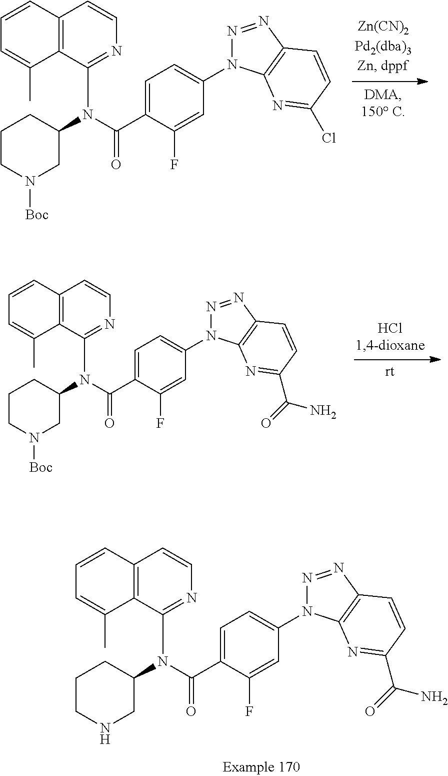 [SMILES: Cc1cccc2ccnc(N(C(=O)c3ccc(-n4nnc5ccc(Cl)nc54)cc3F)[C@@H]3CCCN(C(=O)OC(C)(C)C)C3)c12 ; Cc1cccc2ccnc(N(C(=O)c3ccc(-n4nnc5ccc(C(N)=O)nc54)cc3F)[C@@H]3CCCN(C(=O)OC(C)(C)C)C3)c12 ; Cc1cccc2ccnc(N(C(=O)c3ccc(-n4nnc5ccc(C(N)=O)nc54)cc3F)[C@@H]3CCCNC3)c12]

### Step 1:

To a solution of tert-butyl (R)-3-(4-(5-chloro-3H-[1,2,3]triazolo[4,5-b]pyridin-3-yl)-2-fluoro-N-(8-methylisoquinolin-1-yl)benzamido)piperidine-1-carboxylate (300 mg, 0.487 mmol) in DMA (10 mL) at 25° C. was added Zn(CN)₂ (114.5 mg, 0.975 mmol), Zn (5 mg, 0.058 mmol) and 1,1′-bis(diphenylphosphino) ferrocene (10.8 mg, 0.0195 mmol). The mixture was degassed with nitrogen for 5 min and followed by addition of Pd₂(dba)₃ (17.8 mg, 0.0195 mmol). The reaction mixture was stirred at 120° C. for 16 h before it was cooled. The reaction mixture diluted with EtOAc (100 mL), filtered through celite, and the filtrate was washed with ice water (80 mL). The organic layer was washed with brine, dried over Na₂SO₄, filtered, and concentrated. The residue was purified by Combi-flash using 80% EtOAc in hexane to afford tert-butyl (R)-3-(4-(5-carbamoyl-3H-[1,2,3]triazolo[4,5-b]pyridin-3-yl)-2-fluoro-N-(8-methylisoquinolin-1-yl)benzamido)piperidine-1-carboxylate (150 mg) as a yellow solid. LC-MS: [M+H⁺]=625.35.

### Step 2:

To a solution of (R)-3-(3-fluoro-4-((8-methylisoquinolin-1-yl)(piperidin-3-yl)carbamoyl)phenyl)-3H-[1,2,3]triazolo[4,5-b]pyridine-5-carboxamide (50 mg, 0.080 mmol) in DCM (2 mL) at 0° C. was added 4M HCl in 1,4-dioxane (5 mL). The reaction mixture was stirred at room temperature for 2 h. 2 h before it was concentrated under reduced pressure. The residue was purified by Prep-HPLC (Conditions: Column: SUNFIRE OBD PREP C-18 (250×19×5 μm), Mobile phase A: 0.1% formic acid in water, Mobile phase B: 100% MeCN, Gradient: Time (min)/% B: 0/30, 5/50, 10/100. Flow: 15 mL/min, Dilution: MeCN+H₂O+THF). Pure fractions were combined and dried by lyophilization to afford ((R)-3-(3-fluoro-4-((8-methylisoquinolin-1-yl)(piperidin-3-yl)carbamoyl)phenyl)-3H-[1,2,3]triazolo[4,5-b]pyridine-5-carboxamide formate salt (22 mg) as an off white solid. LC-MS: [M+H⁺]=525.9. ¹H NMR (CD₃OD, 400 MHZ): δ 8.45 (d, J=5.6 Hz, 1H), 8.16 (d, J=9.2 Hz, 1H), 7.91-7.87 (m, 1H), 7.75 (d, J=5.6 Hz, 1H), 7.70-7.67 (m, 2H), 7.55-7.49 (m, 2H), 7.06-7.01 (m, 1H), 6.89 (d, J=9.6 Hz, 1H), 4.57 (m, 1H), 3.59-3.57 (m, 1H), 3.19-3.15 (m, 2H), 2.91-2.89 (m, 1H), 2.80 (s, 3H), 1.59-1.56 (m, 3H), 1.16 (m, 1H).

### Synthesis of (R)-2-fluoro-N-(8-methylisoquinolin-1-yl)-4-(5-phenyl-3H-[1,2,3]triazolo[4,5-b]pyridin-3-yl)-N-(piperidin-3-yl)benzamideamide (Example 171)

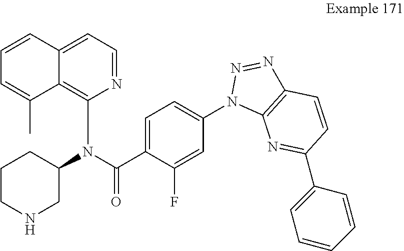 [SMILES: Cc1cccc2ccnc(N(C(=O)c3ccc(-n4nnc5ccc(-c6ccccc6)nc54)cc3F)[C@@H]3CCCNC3)c12]

Example 171 was prepared according to the procedure as described for Example 138 by substituting (1H-pyrazol-3-yl)boronic acid with phenylboronic acid, provided the title compound formic acid salt (30 mg) as an off-white solid. LC-MS: [M+H⁺]=558.2. ¹H NMR (CD₃OD, 400 MHZ): δ 8.42-8.34 (m, 2H), 8.04 (d, J=8.0 Hz, 2H), 7.98-7.89 (m, 2H), 7.79 (d, J=8.4 Hz, 1H), 7.65 (d, J=5.2 Hz, 1H), 7.54-7.37 (m, 6H), 7.05-7.01 (m, 1H), 5.02-4.96 (m, 1H), 3.96-3.92 (m, 1H), 3.78-3.72 (m, 1H), 3.32-3.29 (m, 1H), 2.95-2.88 (m, 1H), 2.79 (s, 3H), 2.93-2.83 (m, 2H), 2.65-2.61 (m, 1H), 1.54-1.51 (m, 1H).

### Synthesis of 2-fluoro-4-(4-methyl-2-oxoimidazolidin-1-yl)-N-(8-methylisoquinolin-1-yl)-N—((R)-piperidin-3-yl)benzamide (Example 172)

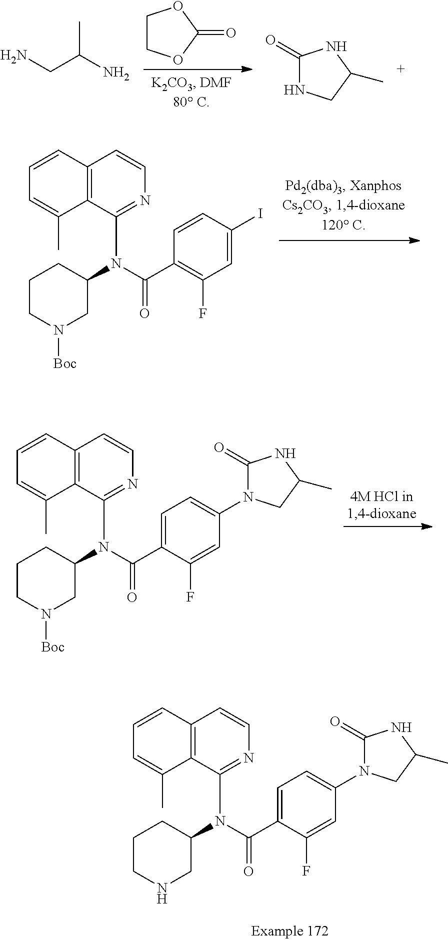

### Step 1:

To a solution of propane-1,2-diamine (500 mg, 6.66 mmol) in DMF (10 mL) at room temperature was added K₂CO₃ (2.7 g, 19.9 mmol) and stirred for 30 min and followed by addition of 1,3-dioxolan-2-one (700 mg, 7.99 mmol). The reaction mixture was stirred at 80° C. for 16 h before it was diluted with water (15 mL) and extracted with EtOAc (2×50 mL). The combined organic layer was washed with brine, dried over Na₂SO₄, and concentrated under reduced pressure to afford 4-methylimidazolidin-2-one (500 mg, impure) as a yellow solid.

### Step 2:

To a solution of tert-butyl (R)-3-(2-fluoro-4-iodo-N-(8-methylisoquinolin-1-yl)benzamido)piperidine-1-carboxylate (200 mg, 0.33 mmol) in 1,4-dioxane (5 mL) at room temperature were added Cs₂CO₃ (321 mg, 0.99 mmol), Xantphos (19 mg, 0.033 mmol) and 5-methyloxazolidin-2-one (68 mg, 0.67 mmol). The reaction mixture was purged with nitrogen for 10 min and followed by addition of Pd₂(dba)₃ (15 mg, 0.016 mmol). The reaction mixture was stirred at 120° C. for 16 h before it was allowed to cool and was filtered through a pad of celite. The filtrate was concentrated under reduced pressure to provide crude residue. The crude residue was diluted with water (30 mL) and extracted with EtOAc (2×50 mL). The combined organic layer was washed with brine, dried over Na₂SO₄ and concentrated under reduced pressure. The crude product was purified by Combi-flash using 50% EtOAc in hexane to afford slightly impure product (100 mg) as a syrup. This material was further purified by Prep-HPLC [Conditions: X-SELECT CSH, Mobile phase A: 0.1% formic acid in water (100%), Mobile phase B: 100% MeCN, Gradient: T/% B: 0/60, 10/85, Flow: 15 mL/min, Dilution: MeCN/H₂O/THF]. Pure fractions were dried by lyophilization to afford tert-butyl (3R)-3-(2-fluoro-4-(4-methyl-2-oxoimidazolidin-1-yl)-N-(8-methylisoquinolin-1-yl)benzamido)piperidine-1-carboxylate (40 mg, 40%) as an off-white solid. LC-MS: [M+H⁺]=562.30.

### Step 3:

To a solution of tert-butyl (3R)-3-(2-fluoro-4-(4-methyl-2-oxoimidazolidin-1-yl)-N-(8-methylisoquinolin-1-yl)benzamido)piperidine-1-carboxylate (40 mg, 0.07 mmol) in DCM (1 mL) at 0° C. was added 4M HCl in 1,4-dioxane (0.5 mL). The reaction mixture was stirred at room temperature for 2 h before it was concentrated reduced pressure. The crude residue was washed with diethyl ether (2×10 mL) to afford crude 2-fluoro-4-(4-methyl-2-oxoimidazolidin-1-yl)-N-(8-methylisoquinolin-1-yl)-N—((R)-piperidin-3-yl)benzamide hydrochloride salt which was further purified by Prep-HPLC [Conditions: Column: Luna OMEGA C-18 (250×21.2×5 μm), Mobile phase A: 0.1% formic acid in water (100%), Mobile phase B: 100% MeCN, Gradient: T/% B: 0/10, 10/50, Flow: 15 mL/min, Dilution: MeCN/H₂O/THF]. Pure fractions were combined and dried by lyophilization to afford 2-fluoro-4-(4-methyl-2-oxoimidazolidin-1-yl)-N-(8-methylisoquinolin-1-yl)-N—((R)-piperidin-3-yl)benzamide hydrochloride salt (20 mg, 66%) as a pale-yellow solid. LC-MS: [M+H⁺]=462.80. ¹H NMR (CD₃OD, 400 MHZ): δ 8.42-8.40 (m, 1H), 7.73-7.63 (m, 2H), 7.52-7.48 (m, 1H), 7.44-7.42 (m, 1H), 7.21-7.18 (m, 1H), 6.71-6.70 (m, 2H), 5.00-4.97 (m, 1H), 3.90-3.85 (m, 1H), 3.78-3.69 (m, 2H), 3.23-3.20 (m, 1H), 2.99-2.82 (m, 1H), 2.79 (s, 3H), 2.63 (s, 2H), 1.87-1.80 (m, 2H), 1.48-1.41 (m, 1H), 1.18 (d, J=4.4 Hz, 1H).

### Synthesis of 2-fluoro-N-(3-methylpyridin-2-yl)-4-morpholino-N-(piperidin-3-yl)benzamide (Example 173)

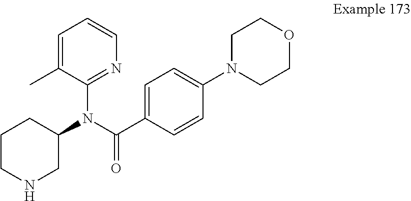 [SMILES: Cc1cccnc1N(C(=O)c1ccc(N2CCOCC2)cc1)[C@@H]1CCCNC1]

Example 173 was prepared according to the procedure described for Example 97 by substituting tert-butyl (R)-3-((6-bromoisoquinolin-1-yl)amino)piperidine-1-carboxylate and 4-(3H-[1,2,3]triazolo[4,5-b]pyridin-3-yl)-2-fluorobenzoyl chloride with 4-morpholinobenzoyl chloride and tert-butyl (R)-3-((3-methylpyridin-2-yl)amino)piperidine-1-carboxylate, provided the title compound hydrochloride salt as an off white solid. LC-MS: [M+H⁺]=399.3.

### Synthesis of (R)-4-(4-methyl-5-oxo-4,5-dihydro-1,3,4-oxadiazol-2-yl)-N-(8 methylisoquinolin-1-yl)-N-(piperidin-3-yl)piperidine-1-carboxamide (Example 174)

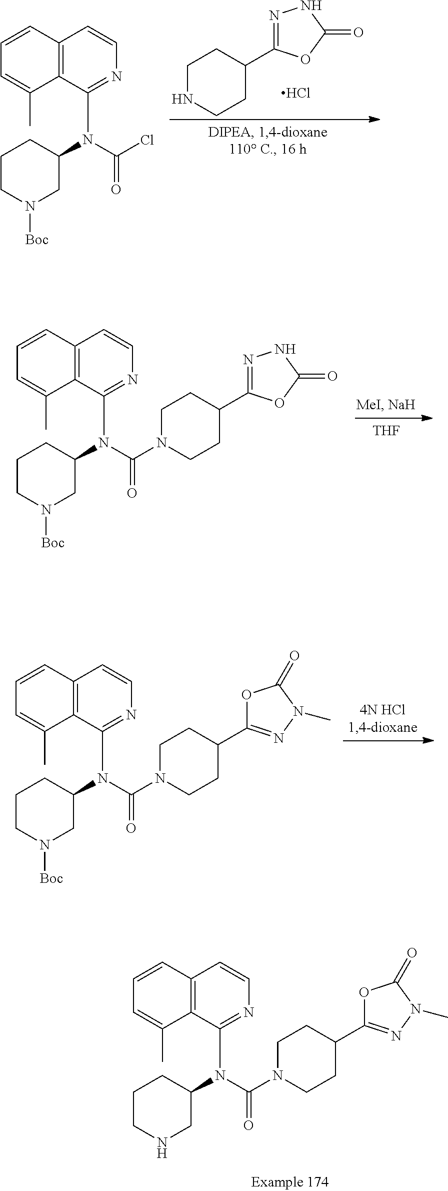

### Step 1:

To a stirred solution of tert-butyl (R)-3-((chlorocarbonyl)(8-methylisoquinolin-1-yl)amino)piperidine-1-carboxylate (250 mg, 0.620 mmol) in 1,4-dioxane (10 mL) at room temperature were added DIPEA (0.540 mL, 3.100 mmol) and 5-(piperidin-4-yl)-1,3,4-oxadiazol-2 (3H)-one hydrochloride (153 mg, 0.744 mmol). The reaction mixture was heated to 110° C. and stirred for 16 h before it was cooled. The reaction mixture was diluted with water (100 mL) and extracted with ethyl acetate (3×100 mL). The combined organic layer was washed with brine (100 mL), dried over Na₂SO₄, and concentrated under reduced pressure. The residue was purified by Combi-flash Using 80-90% EtOAc in hexane to afford tert-butyl (R)-3-(N-(8-methylisoquinolin-1-yl)-4-(5-oxo-4,5-dihydro-1,3,4-oxadiazol-2-yl)piperidine-1-carboxamido)piperidine-1-carboxylate (210 mg, 63%) as an off-white solid. LC-MS: [M+H⁺]=537.3.

### Step 2:

To a solution of tert-butyl (R)-3-(N-(8-methylisoquinolin-1-yl)-4-(5-oxo-4,5-dihydro-1,3,4-oxadiazol-2-yl)piperidine-1-carboxamido)piperidine-1-carboxylate (110 mg, 0.205 mmol) in anhydrous DMF (6 mL) at 0° C. was added NaH (60% in mineral oil, 20 mg, 0.512 mmol). The reaction mixture was stirred for 15 min, after which iodomethane (0.038 mL, 0.615 mmol) was added. The reaction mixture was warmed and allowed to stir at room temperature for 4 h before it was quenched with saturated aq. NH₄Cl (100 mL), and extracted with ethyl acetate (2×150 mL). The organic layer was washed with brine (150 mL), dried over Na₂SO₄, and concentrated under reduced pressure. The residue was purified by Combi-flash using 90% EtOAc in hexane. The residue was further purified by Prep-HPLC (Conditions: Column: Luna OMEGA C-18 (250×21.2×5 μm), Mobile phase A: 0.1% formic acid in water (100%), Mobile phase B: 100% MeCN, Gradient: T/% B: 0/70, 10/80, Flow: 15 mL/min, Dilution: MeCN+H₂O+THF). Pure fractions were dried by lyophilization to afford tert-butyl (R)-3-(4-(4-methyl-5-oxo-4,5-dihydro-1,3,4-oxadiazol-2-yl)-N-(8-methylisoquinolin-1-yl)piperidine-1-carboxamido)piperidine-1-carboxylate (55 mg, 69%) as an off-white solid. LC-MS: [M+H⁺]=551.3.

### Step 3:

The title compound hydrochloride salt was obtained as an off-white solid by deprotection Method A used in Example 97. LC-MS: [M+H⁺]=451.95. ¹H NMR (CD₃OD, 400 MHZ): δ 8.38-8.28 (m, 1H), 7.86-7.81 (m, 1H), 7.76-7.75 (m, 1H), 7.69-7.63 (m, 1H), 7.59-7.50 (m, 1H), 4.85-4.81 (m, 1H), 4.10-4.05 (m, 1H), 3.70-3.65 (m, 2H), 3.47-3.35 (m, 1H), 3.22 (m, 3H), 3.07-3.06 (m, 1H), 2.93-2.90 (m, 2H), 2.69 (s, 3H), 2.51-2.47 (m, 1H), 1.97-1.80 (m, 3H), 1.57-1.55 (m, 1H), 1.28-1.20 (m, 2H), 1.09-1.00 (m, 2H), 0.42-0.34 (m, 1H).

### Synthesis of (R)-4-(3H-[1,2,3]triazolo[4,5-b]pyridin-3-yl)-N-(5,5-difluoropiperidin-3-yl)-2-fluoro-N-(8-methylisoquinolin-1-yl)benzamide (Example 175)

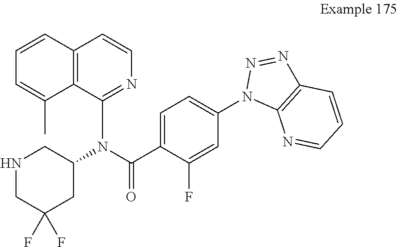 [SMILES: Cc1cccc2ccnc(N(C(=O)c3ccc(-n4nnc5cccnc54)cc3F)[C@H]3CNCC(F)(F)C3)c12]

Example 175 was prepared according to the procedure described for Example 241 by substituting (R)-5-amino-piperidine-1-carboxylate with (R)-5-amino-3,3-difluoropiperidine-1-carboxylate, provided the title compound hydrochloride acid salt as an off-white solid. LC-MS: [M+H⁺]=518.0. ¹H NMR (400 MHZ, DMSO-d₆): δ 8.85-8.78 (m, 1H), 8.68 (dd, J=1.2, 8.3 Hz, 1H), 8.52-8.46 (m, 1H), 7.96 (dd, J=1.8, 11.2 Hz, 1H), 7.86 (d, J=5.5 Hz, 1H), 7.81-7.73 (m, 2H), 7.64-7.52 (m, 3H), 7.17 (q, J=8.4 Hz, 1H), 5.17-4.95 (m, 1H), 3.79-3.56 (m, 2H), 3.17-3.03 (m, 1H), 3.01-2.91 (m, 1H), 2.87-2.79 (m, 3H), 2.46-2.39 (m, 1H), 2.24-2.06 (m, 1H).

### Synthesis of (R)—N-(3-(1H-pyrazol-4-yl)pyridin-2-yl)-4-(3H-[1,2,3]triazolo[4,5-b]pyridin-3-yl)-2-fluoro-N-(piperidin-3-yl)benzamide (Example 176)

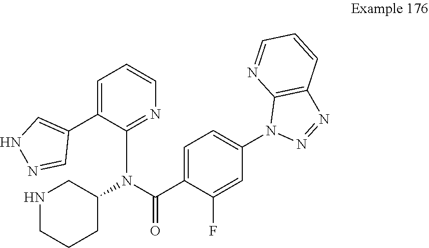 [SMILES: O=C(c1ccc(-n2nnc3cccnc32)cc1F)N(c1ncccc1-c1cn[nH]c1)[C@@H]1CCCNC1]

Example 176 was prepared according to the procedure described for Example 104 by substituting tert-butyl-dimethyl-[(E)-4-(4,4,5,5-tetramethyl-1,3,2-dioxaborolan-2-yl)but-3-enoxy]silane with (1-(tert-butoxycarbonyl)-1H-pyrazol-4-yl)boronic acid, provided the title compound hydrochloride salt as an off white solid. LC-MS: [M+H⁺]=484.1. ¹H NMR (400 MHz, DMSO-d₆): δ 1.17-1.36 (m, 1H), 1.73-1.85 (m, 2H), 1.86-2.00 (m, 1H), 2.76 (br d, J=12.26 Hz, 1H), 3.10-3.36 (m, 2H), 3.55-3.65 (m, 1H), 4.77-4.97 (m, 1H), 5.07 (br t, J=11.13 Hz, 1H), 6.75-6.98 (m, 1H), 7.37 (br dd, J=7.25, 4.75 Hz, 1H), 7.63 (br dd, J=8.13, 4.38 Hz, 1H), 7.80-7.90 (m, 3H), 7.90-8.03 (m, 2H), 8.39-8.51 (m, 1H), 8.71 (br d, J=8.13 Hz, 1H), 8.86 (br d, J=3.88 Hz, 1H), 9.08 (br d, J=11.38 Hz, 1H), 9.46 (br d, J=10.13 Hz, 1H).

### Synthesis of (R)-4-(3H-[1,2,3]triazolo[4,5-b]pyridin-3-yl)-2-fluoro-N-(piperidin-3-yl)-N-(3-(thiophen-3-yl)pyridin-2-yl)benzamide (Example 177)

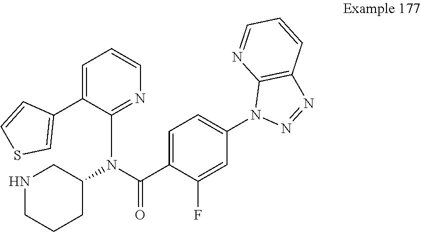 [SMILES: O=C(c1ccc(-n2nnc3cccnc32)cc1F)N(c1ncccc1-c1ccsc1)[C@@H]1CCCNC1]

Example 177 was prepared according to the procedure described for Example 104 by substituting tert-butyl-dimethyl-[(E)-4-(4,4,5,5-tetramethyl-1,3,2-dioxaborolan-2-yl)but-3-enoxy]silane with thiophen-3-ylboronic acid, provided the title compound hydrochloride salt as an off white solid. LC-MS: [M+H⁺]=500.1. ¹H NMR (400 MHZ, methanol-d₄): δ 8.76-8.94 (m, 1H), 8.47-8.68 (m, 2H), 7.91-8.12 (m, 2H), 7.82 (dd, J=7.75, 1.50 Hz, 1H), 7.53-7.67 (m, 2H), 7.32-7.47 (m, 2H), 7.13 (br d, J=5.25 Hz, 1H), 6.69 (t, J=7.82 Hz, 1H), 5.07-5.27 (m, 1H), 3.77-3.99 (m, 2H), 3.43 (br d, J=13.01 Hz, 1H), 3.02 (td, J=12.07, 3.88 Hz, 1H), 2.59-2.73 (m, 1H), 2.37 (br dd, J=12.13, 1.88 Hz, 1H), 1.91-2.19 (m, 3H), 1.81-1.90 (m, 1H), 1.64-1.79 (m, 1H).

### Synthesis of 8a-methyl-N-(8-methylisoquinolin-1-yl)-3-oxo-N—((R)-piperidin-3-yl)tetrahydro-3H-oxazolo[3,4-a]pyrazine-7 (1H)-carboxamide (Example 178)

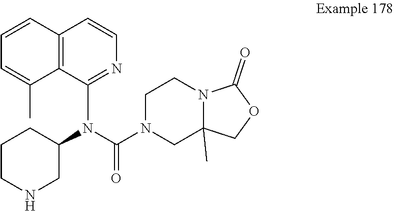 [SMILES: Cc1cccc2ccnc(N(C(=O)N3CCN4C(=O)OCC4(C)C3)[C@@H]3CCCNC3)c12]

Example 178 was prepared according to the procedure described for Example 243 by substituting 3-methyl-5-(piperidin-4-yl)isoxazole with 8a-methylhexahydro-3H-oxazolo[3,4-a]pyrazin-3-one and using 4M HCl in dioxane for removal of the tert-butyloxycarbonyl group, provided the title compound as an off-white solid. LC-MS: [M+H⁺]=424.2.

### Synthesis of (R)-4-(5-methyl-2H-tetrazol-2-yl)-N-(8-methylisoquinolin-1-yl)-N-(piperidin-3-yl)benzamide (Example 179)

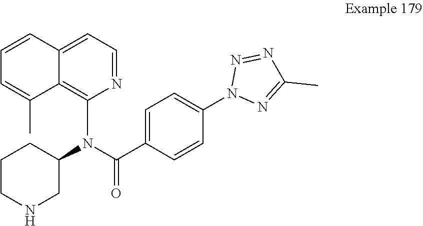 [SMILES: Cc1nnn(-c2ccc(C(=O)N(c3nccc4cccc(C)c34)[C@@H]3CCCNC3)cc2)n1]

Example 179 was prepared according to the procedure described for Example 141 by substituting propionimidamide with acetimidamide, provided the title compound as an off-white solid. LC-MS: [M+H⁺]=428.2. ¹H NMR (CD₃OD, 400 MHZ): δ 8.42 (d, J=5.6 Hz, 1H), 7.72 (d, J=5.6 Hz, 1H), 7.62-7.55 (m, 3H), 7.39-7.35 (m, 1H), 7.31-7.28 (m, 1H), 7.15 (d, J=8.8 Hz, 2H), 5.04-4.98 (m, 1H), 3.86 (m, 1H), 3.75-3.69 (m, 1H), 3.31-3.28 (m, 1H), 2.93-2.89 (m, 1H), 2.66 (s, 3H), 2.40 (s, 3H), 1.84-1.80 (m, 3H), 1.44 (m, 1H).

### Synthesis of (R)-2-fluoro-4-(methyl (4-methylpyrimidin-2-yl)amino)-N-(8-methylisoquinolin-1-yl)-N-(piperidin-3-yl)benzamide (Example 180)

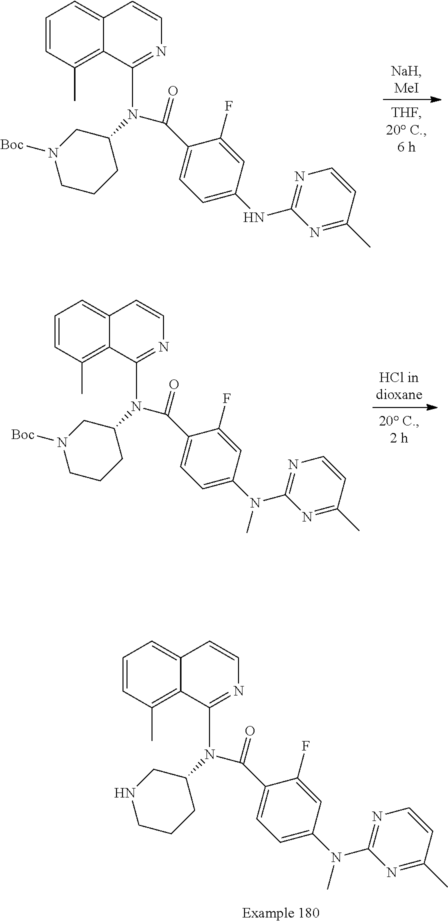 [SMILES: Cc1ccnc(Nc2ccc(C(=O)N(c3nccc4cccc(C)c34)[C@@H]3CCCN(C(=O)OC(C)(C)C)C3)c(F)c2)n1 ; Cc1ccnc(N(C)c2ccc(C(=O)N(c3nccc4cccc(C)c34)[C@@H]3CCCN(C(=O)OC(C)(C)C)C3)c(F)c2)n1 ; Cc1ccnc(N(C)c2ccc(C(=O)N(c3nccc4cccc(C)c34)[C@@H]3CCCNC3)c(F)c2)n1]

### Step 1:

To a solution of tert-butyl (R)-3-(2-fluoro-N-(8-methylisoquinolin-1-yl)-4-((4-methylpyrimidin-2-yl)amino)benzamido)piperidine-1-carboxylate (25.00 mg, 43.81 μmol) in THF (1.5 mL) at 0° C. under N₂ atmosphere was added NaH (60% in mineral oil, 5.26 mg, 131.43 μmol). The mixture was stirred at 20° C. for 0.5 h and followed by addition of iodomethane (24.87 mg). The mixture was stirred at 20° C. for 5.5 h before it was poured into water (1 mL). The mixture was partitioned between ethyl acetate (2 mL) and water (2 mL). The combined organic layer was washed with brine, dried over Na₂SO₄, and concentrated. The residue was purified by Prep-TLC to give tert-butyl (R)-3-(2-fluoro-4-(methyl (4-methylpyrimidin-2-yl)amino)-N-(8-methylisoquinolin-1-yl)benzamido)piperidine-1-carboxylate (20.00 mg, 78%) as a red solid. LC-MS (ESI+): m/z 585.4 [M+H⁺].

### Step 2:

The title compound hydrochloride salt was obtained as an off-white solid by deprotection Method A used in Example 97. LC-MS (ESI+): m/z 485.3 (M+H⁺). ¹H NMR (400 MHZ, DMSO-d₆) δ 9.39-8.97 (m, 2H), 8.48-8.39 (m, 1H), 8.20 (d, J=5.0 Hz, 1H), 7.86-7.79 (m, 1H), 7.79-7.73 (m, 1H), 7.56 (t, J=7.6 Hz, 1H), 7.48 (br d, J=7.0 Hz, 1H), 7.03-6.94 (m, 1H), 6.76-6.66 (m, 3H), 4.99-4.77 (m, 1H), 3.76 (br d, J=12.9 Hz, 1H), 3.59-3.48 (m, 1H), 3.33-3.16 (m, 4H), 2.88-2.72 (m, 4H), 2.22 (s, 3H), 1.89-1.69 (m, 3H), 1.33-1.14 (m, 3H), 0.91-0.69 (m, 1H).

### Synthesis of (R)—N-(azepan-3-yl)-N-(8-methylisoquinolin-1-yl)-4-(3-methylisoxazol-5-yl)piperidine-1-carboxamide (Example 181)

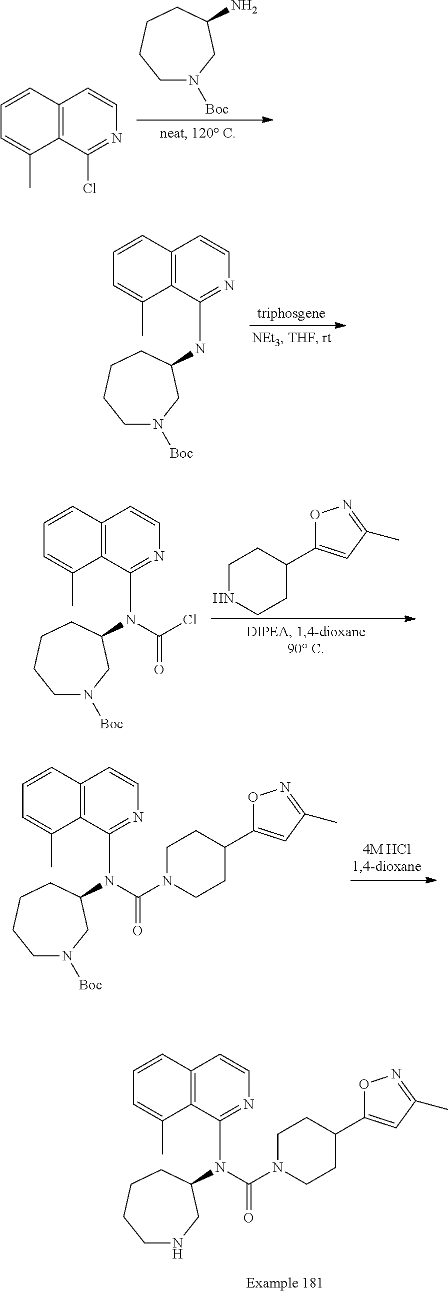

### Step 1:

A mixture of methyl 1-chloro-8-methylisoquinoline (500 mg, 2.814 mmol) and tert-butyl (R)-3-aminoazepane-1-carboxylate (1.2 g, 5.629 mmol) in a sealed tube was stirred at 120° C. for 16 h before it was allowed to cool. The reaction mixture was quenched with ice cold water and extracted with EtOAc (2×50 mL). The combined organic layer was washed with brine, dried over on Na₂SO₄ and concentrated. The crude product was purified by Combi-flash using 30% EtOAc in hexane to afford tert-butyl (R)-3-((8-methylisoquinolin-1-yl)amino)azepane-1-carboxylate (700 mg, 70%) as a colorless gummy solid. LC-MS: [M+H⁺]=356.3.

### Step 2:

To a solution of tert-butyl (R)-3-((8-methylisoquinolin-1-yl)amino)azepane-1-carboxylate (450 mg, 1.265 mmol) in THF (20 mL) at −65° C. were added Et₃N (1.06 mL, 7.595 mmol) and triphosgene (630 mg, 3.797 mmol). The reaction mixture was stirred at room temperature for 16 h before it was diluted with saturated aq. NH₄Cl solution (30 mL) and extracted with ethyl acetate (2×100 mL). The combined organic layer was washed with brine, dried over Na₂SO₄, and concentrated. The crude product was purified by Combi-flash using 30% EtOAc in hexane to afford tert-butyl (R)-3-((chlorocarbonyl)(8-methylisoquinolin-1-yl)amino)azepane-1-carboxylate (400 mg, 77%) as a colorless gummy solid. LC-MS: [M+H⁺]=417.9.

### Step 3:

To a solution of tert-butyl (R)-3-((chlorocarbonyl)(8-methylisoquinolin-1-yl)amino)azepane-1-carboxylate (200 mg, 0.479 mmol) in 1,4-dioxane (10 mL) at room temperature were added 3-methyl-5-(piperidin-4-yl)isoxazole (110 mg, 0.575 mmol) and DIPEA (0.25 mL, 1.438 mmol). The reaction mixture was stirred at 90° C. for 16 h before it was diluted with ice cold water (30 mL) and extracted with ethyl acetate (2×50 mL). The combined organic layer was washed with brine, dried over Na₂SO₄, and concentrated under reduced pressure. The crude product was purified by Combi-flash using 40% EtOAc in hexane to afford tert-butyl (R)-3-(N-(8-methylisoquinolin-1-yl)-4-(3-methylisoxazol-5-yl)piperidine-1-carboxamido)azepane-1-carboxylate (80 mg, 31%) as an off-white solid. LC-MS: [M+H⁺]=548.4.

### Step 4:

The title compound hydrochloride salt was obtained as an off-white solid by deprotection Method A used in Example 97. LC-MS: [M+H⁺]=448.2. ¹H NMR (CD₃OD, 400 MHZ) δ 8.34 (d, J=5.6 Hz, 1H), 7.84-7.82 (m, 1H), 7.79-7.74 (m, 1H), 7.71-7.65 (m, 1H), 7.54-7.52 (m, 1H), 5.73 (s, 1H), 4.83-4.79 (m, 1H), 3.97-3.94 (m, 1H), 3.89-3.79 (m, 1H), 3.62-3.59 (m, 3H), 3.05-2.98 (m, 4H), 2.71 (s, 3H), 2.64-2.56 (m, 4H), 2.30-2.29 (m, 1H), 2.14 (s, 3H), 1.98-1.97 (m, 2H), 1.86-1.49 (3H).

### Synthesis of (R)—N-(8-chloroisoquinolin-1-yl)-2-fluoro-4-(3-methylpyrazin-2-yl)-N-(piperidin-3-yl)benzamide (Example 182)

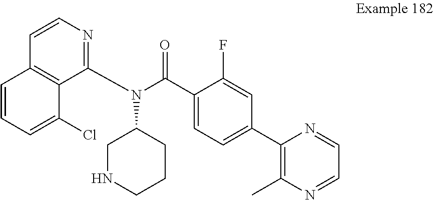 [SMILES: Cc1nccnc1-c1ccc(C(=O)N(c2nccc3cccc(Cl)c23)[C@@H]2CCCNC2)c(F)c1]

Example 182 was prepared according to the procedure described for Example 183 by substituting 2,3-dichloropyrazine with 2-chloro-3-methyl-pyrazine, provided the title compound as hydrochloride salt. LC-MS: [M+H⁺]=476.2. ¹H NMR (400 MHZ, DMSO-d₆) δ 1.40-1.59 (m, 1H), 1.81-2.24 (m, 4H), 2.32 (s, 3H), 2.59-2.80 (m, 1H) 2.93-3.20 (m, 2H), 3.41 (br d, J=12.38 Hz, 1H), 3.73-3.84 (m, 1H), 3.97-4.05 (m, 1H), 5.06-5.18 (m, 1H), 6.99-7.06 (m, 2H), 7.27 (t, J=7.44 Hz, 1H), 7.77 (dd, J=7.44, 0.81 Hz, 1H), 7.81-7.86 (m, 2H), 8.51-8.55 (m, 2H), 8.60 (d, J=2.50 Hz, 1H).

### Synthesis of (R)—N-(8-chloroisoquinolin-1-yl)-4-(3-chloropyrazin-2-yl)-2-fluoro-N-(piperidin-3-yl)benzamide (Example 183)

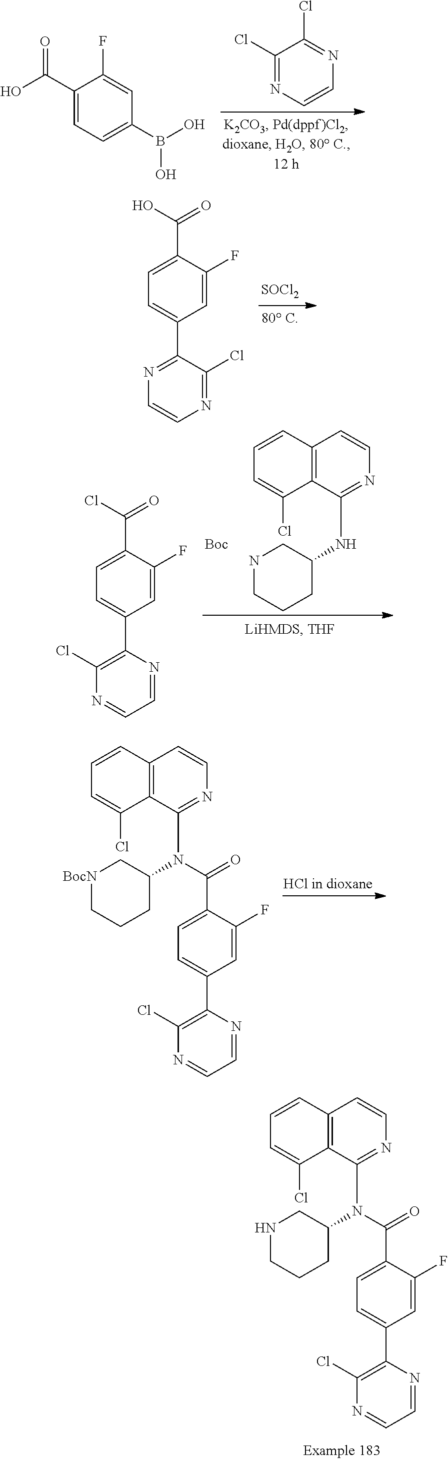

### Step 1:

To a solution of 2, 3-dichloropyrazine (0.50 g, 3.36 mmol) in 1,4-dioxane (10 mL) and H₂O (2 mL) was added 4-borono-2-fluorobenzoic acid (308.65 mg, 1.68 mmol), Na₂CO₃ (1.07 g, 10.07 mmol) and Pd(dppf)Cl₂ (245.57 mg, 335.62 μmol) at 20° C. under N₂ atmosphere. The reaction was stirred at 80° C. for 2 h. The reaction mixture was filtered, and the filtrate was concentrated under reduced pressure to give a residue. This residue was purified by flash column chromatography (SiO₂, petroleum ether/ethyl acetate=5/1 to 3/1) to provide 4-(3-chloropyrazin-2-yl)-2-fluorobenzoic acid (0.60 g, 27%) as a white solid. LC-MS: [M+H⁺]=251.6.

### Step 2:

To a solution of 4-(3-chloropyrazin-2-yl)-2-fluorobenzoic acid (0.20 g, 791.68 μmol) in SOCl₂ (2 mL) was added DMF (3.05 μL, 39.58 μmol) at 20° C. under N₂ atmosphere. The reaction was stirred at 80° C. for 2 h before it was concentrated under reduced pressure to give 4-(3-chloropyrazin-2-yl)-2-fluorobenzoyl chloride (0.135 g, 63% yield) as a yellow solid.

### Step 3:

To a solution of tert-butyl (R)-3-((8-chloroisoquinolin-1-yl)amino)piperidine-1-carboxylate (151.69 mg, 559.60 μmol) in tetrahydrofuran (5 mL) at 0° C. under N₂ atmosphere was added 1M LiHMDS in THF (1.12 mL). The mixture was stirred at 0° C. for 0.5 h and followed by addition of 4-(3-chloropyrazin-2-yl)-2-fluorobenzoyl chloride (0.135 g, 373.07 μmol). The reaction was stirred at 20° C. for 0.5 h before it was poured into saturated aq. NH₄Cl solution (6 mL). The aqueous phase was extracted with ethyl acetate (3×5 mL). The combined organic phase was washed with brine (5 mL), dried over anhydrous Na₂SO₄, concentrated. This residue was then purified by prep-HPLC (column: Phenomenex Luna C18 75×30 mm×3 μm; mobile phase: [H₂O (0.04% HCl)-MeCN]; gradient: 50%-80% B over 8.0 min) to give tert-butyl (R)-3-(N-(8-chloroisoquinolin-1-yl)-4-(3-chloropyrazin-2-yl)-2-fluorobenzamido)piperidine-1-carboxylate (80 mg, 36%) as a white solid. LC-MS: [M+H⁺]=540.3.

### Step 4:

The title compound hydrochloride salt was obtained as a white solid by deprotection Method A used in Example 97. LC-MS: [M+H⁺]=496.1. ¹H NMR (400 MHZ, DMSO-d₆) δ 1.15-1.31 (m, 1H), 1.74-2.06 (m, 3H), 2.62-2.92 (m, 1H), 3.17-3.60 (m, 2H), 3.78 (br d, J=11.63 Hz, 1H), 4.86-5.08 (m, 1H), 7.06-7.27 (m, 3H), 7.52-8.03 (m, 5H), 8.39-8.73 (m, 3H), 9.14 (br d, J=10.51 Hz, 1H), 9.27-9.49 (m, 1H).

### Synthesis of (R)—N-(8-methylisoquinolin-1-yl)-N-(piperidin-3-yl)-5-(pyrimidin-2-ylamino) picolinamide (Example 184)

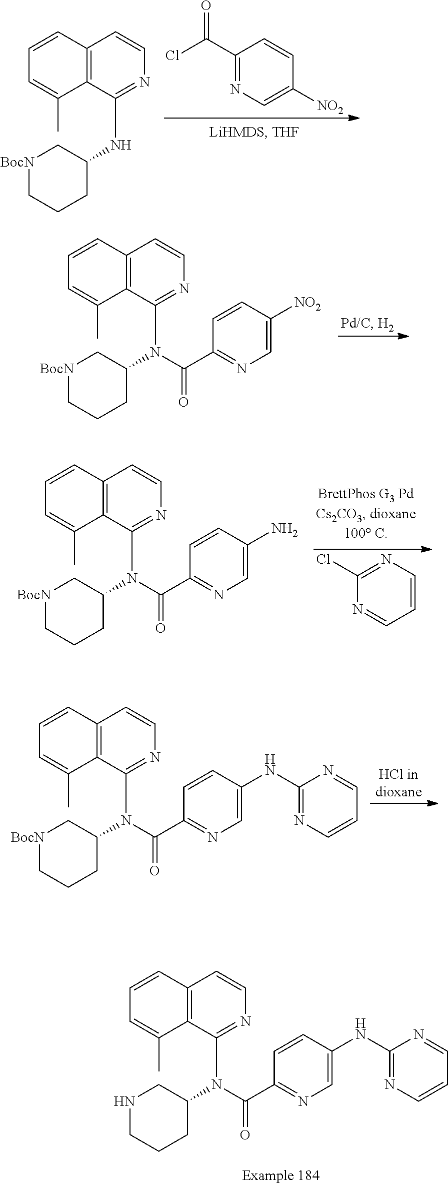

### Step 1:

To a solution of tert-butyl (R)-3-((8-methylisoquinolin-1-yl)amino)piperidine-1-carboxylate (732.1 mg, 2.14 mmol) in THF (10 mL) was added LiHMDS in THF (1 M, 6.43 mL) at 0° C. under N₂. The mixture was stirred at 0° C. for 0.5 h and followed by addition of 5-nitropicolinoyl chloride (800 mg, 4.29 mmol) in THF (5 mL). The reaction was stirred at 20° C. for 0.5 h before it was poured into water (100 mL), extracted with ethyl acetate (3×50 mL×3), washed with brine and dried over sodium sulfate. The combined organic layer was concentrated under reduced pressure. The residue was purified by prep-HPLC (column: Phenomenex luna C18 100×40 mm×5 μm; mobile phase: [H₂O (0.1% TFA)-MECN]; gradient: 35%-65% B over 8.0 min) to give tert-butyl (R)-3-(N-(8-methylisoquinolin-1-yl)-5-nitropicolinamido)piperidine-1-carboxylate (150 mg, 14%) as a yellow solid. LC-MS (ESI+): 492.3 [M+H⁺].

### Step 2:

To a mixture of tert-butyl (R)-3-(N-(8-methylisoquinolin-1-yl)-5-nitropicolinamido)piperidine-1-carboxylate (150 mg, 305.16 μmol) and Pd/C (64.95 mg, 305.16 μmol, 50 wt %) in THF (10 mL) was degassed and purged with H₂ (15 Psi) thrice. Then the mixture was stirred at 25° C. for 12 h. The mixture was filtered, and the filtrate was concentrated under reduced pressure to afford crude tert-butyl (R)-3-(5-amino-N-(8-methylisoquinolin-1-yl)picolinamido)piperidine-1-carboxylate (140 mg, 99%) as a white solid which was used in the next step without further purification. LCMS (ESI+): 462.3 [M+H⁺].

### Step 3:

To a solution of tert-butyl (R)-3-(5-amino-N-(8-methylisoquinolin-1-yl)picolinamido)piperidine-1-carboxylate (100.00 mg, 169.65 μmol) and 2-chloropyrimidine (37.03 mg, 339.31 μmol) in dioxane (1 mL) was added Cs₂CO₃ (165.83 mg, 508.96 μmol) and BrettPhos G3 Pd(30.76 mg, 33.93 μmol) at 20° C. under N₂. The mixture was stirred at 100° C. for 2 h before it was cooled. The mixture was filtered and the filtrate was purified by prep-HPLC (column: Phenomenex Luna C18 75*30 mm*3 μm; mobile phase: [H₂O (0.04% HCl)-MeCN]; gradient: 50%-80% B over 8.0 min) to give tert-butyl (R)-3-(N-(8-methylisoquinolin-1-yl)-5-(pyrimidin-2-ylamino)picolinamido)piperidine-1-carboxylate (70.00 mg, 69%) as a white solid. LC-MS (ESI+): 540.4 [M+H⁺].

### Step 4:

To a mixture of tert-butyl (R)-3-(N-(8-methylisoquinolin-1-yl)-5-(pyrimidin-2-ylamino)picolinamido)piperidine-1-carboxylate (25 mg, 46.33 μmol) in dioxane (3 mL) was added 4M HCl in dioxane (3 mL). Then the mixture was stirred at 20° C. for 0.5 h before it was concentrated under reduced pressure. The crude product was purified by Prep-HPLC (column: Phenomenex Luna 80*30 mm*3 μm; mobile phase: [H₂O (0.04% HCl)-MeCN]; gradient: 5%-50% B over 8.0 min) to give (R)—N-(8-methylisoquinolin-1-yl)-N-(piperidin-3-yl)-5-(pyrimidin-2-ylamino) picolinamide hydrochloride salt (15.7 mg, 75% yield) as an off-white solid. LC-MS (ESI+): m/z 440.3 [M+H⁺]. ¹H NMR (400 MHZ, DMSO-d₆) δ 0.85 (qd, J=12.19, 4.69 Hz, 1H), 1.55-1.81 (m, 3H), 2.60-2.88 (m, 4H), 3.14 (br d, J=11.88 Hz, 1H), 3.30 (q, J=10.67 Hz, 1H), 3.72 (br d, J=11.38 Hz, 1H), 4.73-4.88 (m, 1H), 6.91 (t, J=4.82 Hz, 1H), 7.45 (d, J=7.00 Hz, 1H), 7.60 (t, J=7.69 Hz, 1H), 7.76-7.89 (m, 3H), 8.03 (d, J=2.38 Hz, 1H), 8.22 (dd, J=8.76, 2.63 Hz, 1H), 8.29 (d, J=5.50 Hz, 1H), 8.48 (d, J=4.88 Hz, 2H), 9.01-9.16 (m, 1H), 9.37 (br d, J=10.13 Hz, 1H), 9.92 (s, 1H).

### Synthesis of (R)—N-(azepan-3-yl)-2-fluoro-N-(8-methylisoquinolin-1-yl)-4-(pyrimidin-2-ylamino)benzamide (Example 185)

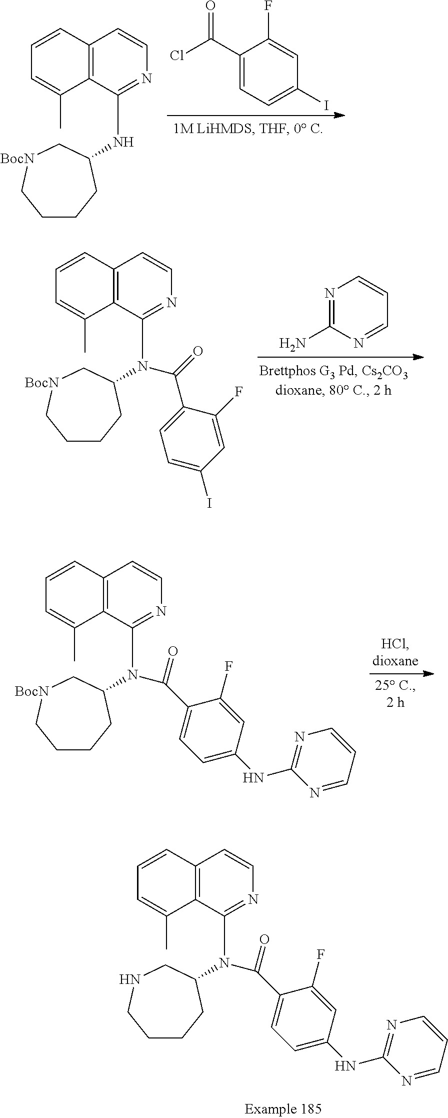

Example 185 was prepared according to the procedure described for Example 210 by substituting 1-chloro-8-methylisoquinoline with tert-butyl (R)-3-aminoazepane-1-carboxylate, provided the title compound hydrochloride salt as a yellow solid. LC-MS (ESI+): m/z 471.4 [M+H⁺]. ¹H NMR (400 MHZ, methanol-d₄) δ 1.59-1.89 (m, 4H), 2.01-2.13 (m, 1H), 2.28-2.39 (m, 1H), 2.80 (s, 3H), 3.17-3.26 (m, 1H), 3.58-3.66 (m, 1H), 3.72-3.82 (m, 1H), 4.00 (dd, J=14.43, 4.77 Hz, 1H), 5.39-5.48 (m, 1H), 6.78-6.85 (m, 1H), 6.90 (dd, J=8.53, 1.76 Hz, 1H), 7.05 (t, J=5.14 Hz, 1H), 7.35 (dd, J=12.80, 1.63 Hz, 1H), 7.43-7.48 (m, 1H), 7.52 (t, J=7.65 Hz, 1H), 7.67 (d, J=8.03 Hz, 1H), 7.79 (d, J=5.52 Hz, 1H), 8.47 (d, J=5.52 Hz, 1H), 8.57 (d, J=5.14 Hz, 2H). ¹⁹F NMR (376 MHz, methanol-d₄) δ −112.516 (s, 1F).

### Synthesis of (R)-2-fluoro-N-(8-methylisoquinolin-1-yl)-4-((5-methylpyrimidin-2-yl)amino)-N-(piperidin-3-yl)benzamide (Example 186)

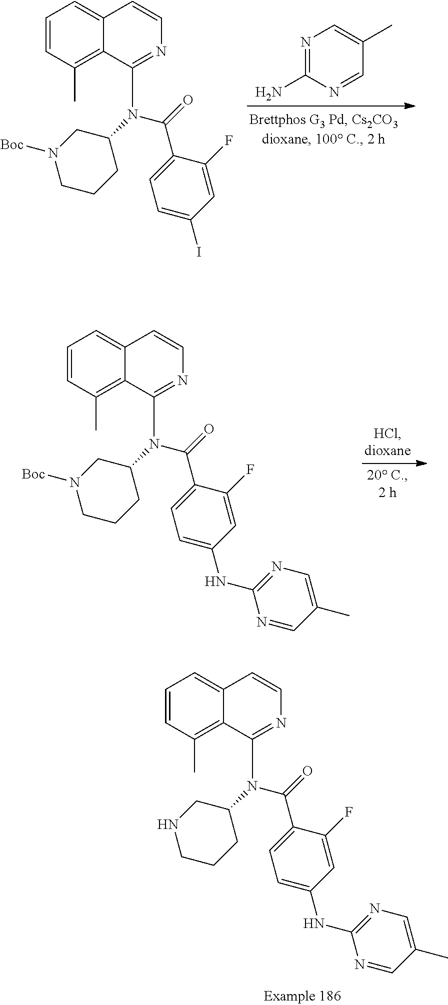

Example 186 was prepared according to the procedure described for Example 185 by substituting pyrimidin-2-amine with 5-methylpyrimidin-2-amine, provided the title compound hydrochloride salt as a white solid. LC-MS (ESI+): m/z 471.3 [M+H⁺]. ¹H NMR (400 MHZ, methanol-d₄) δ 8.43 (d, J=5.5 Hz, 1H), 8.33 (s, 2H), 7.77-7.70 (m, 1H), 7.69-7.55 (m, 1H), 7.53-7.38 (m, 3H), 6.81 (dd, J=2.0, 8.6 Hz, 1H), 6.66 (t, J=8.3 Hz, 1H), 5.14-5.00 (m, 1H), 3.96 (br dd, J=3.3, 12.0 Hz, 1H), 3.87-3.76 (m, 1H), 3.40 (br d, J=12.4 Hz, 1H), 3.10-2.92 (m, 1H), 2.88-2.77 (m, 3H), 2.75-2.58 (m, 1H), 2.19 (s, 3H), 2.03-1.83 (m, 3H), 1.65-1.50 (m, 1H).

### Synthesis of (R)-2-fluoro-4-(5-methoxy-3H-[1,2,3]triazolo[4,5-b]pyridin-3-yl)-N-(8-methylisoquinolin-1-yl)-N-(piperidin-3-yl)benzamide (Example 187)

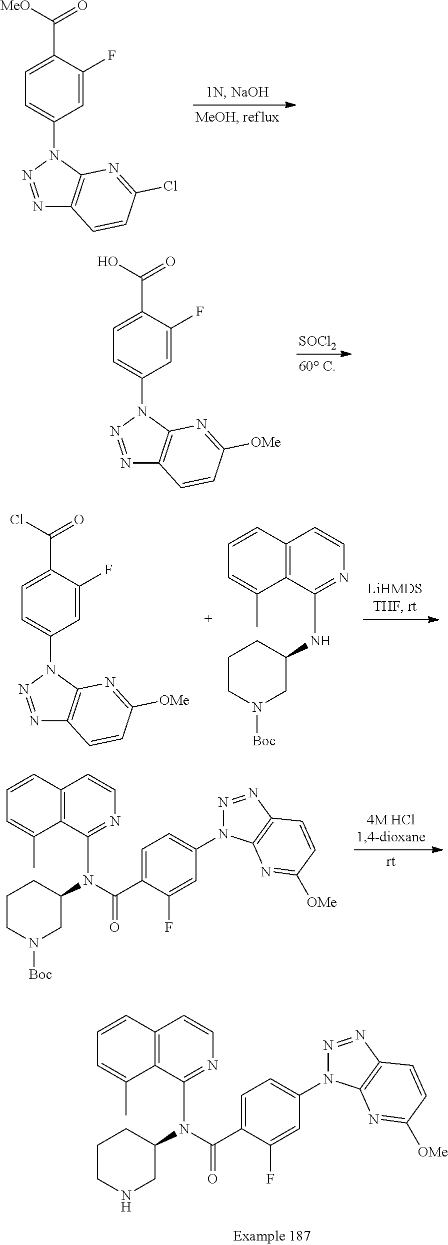

Step 1: To a solution of methyl 4-(5-chloro-3H-[1,2,3]triazolo[4,5-b]pyridin-3-yl)-2-fluorobenzoate (600 mg, 1.96 mmol) in methanol (8 mL) was added NaOH (392 mg, 9.80 mmol) and H₂O (5 mL) at 0° C. The reaction mixture was gradually warmed at 70° C. for 16 h. The reaction mixture was concentrated under reduced pressure, diluted with water (20 mL), and acidified with 1N aq. HCl to pH 2. The precipitate was collected by filtration and dried to afford crude 2-fluoro-4-(5-methoxy-3H-[1,2,3]triazolo[4,5-b]pyridin-3-yl)benzoic acid (500 mg) as pale-yellow solid. LC-MS: [M−H⁻]=287.0.

### Step 2:

To a solution of 2-fluoro-4-(5-methoxy-3H-[1,2,3]triazolo[4,5-b]pyridin-3-yl)benzoic acid (420 mg, 1.45 mmol) in SOCl₂ (10 mL) was added a catalytic amount of DMF (0.1 mL) at 0° C. and the reaction mixture was stirred for 16 h at 90° C. The reaction mixture was concentrated under reduced pressure, further co-distilled with toluene, and dried to afford crude 2-fluoro-4-(5-methoxy-3H-[1,2,3]triazolo[4,5-b]pyridin-3-yl)benzoyl chloride (500 mg, crude).

### Step 3:

To a solution of tert-butyl (R)-3-((8-methylisoquinolin-1-yl)amino)piperidine-1-carboxylate (200 mg, 0.586 mmol) in THF (10 mL) was added 1.5M LiHMDS in THF (1.1 mL, 1.75 mmol) at 0° C. and stirred for 10 min and followed by addition of crude 2-fluoro-4-(5-methoxy-3H-[1,2,3]triazolo[4,5-b]pyridin-3-yl)benzoyl chloride (287 mg, 0.938 mmol) in THF (5 mL). The reaction mixture was stirred for 30 min before it was diluted with H₂O and extracted with EtOAc (50 mL). The organic layer was washed with brine, dried over Na₂SO₄, and concentrated under reduced pressure. The residue was purified by flash column chromatography and was eluted with 80% EtOAc in hexane and then further purified by Prep-HPLC (Conditions: Column: X-SELECT CSH C18 (250*19) mm, 5 μm, Mobile phase A: 0.1% formic acid in water, Mobile phase B: 100% MeCN, Gradient: Time (min)/% B: 0/70, 10/90, Flow: 15 mL/min, Dilution: MeCN+H₂O+THF). Pure fractions were combined and dried by lyophilization to afford tert-butyl (R)-3-(2-fluoro-N-(8-methylisoquinolin-1-yl)-4-(1H-1,2,3-triazol-1-yl)benzamido)piperidine-1-carboxylate (100 mg) as an off-white solid. LC-MS: [M+H⁺]=612.0.

### Step 4:

To a solution of tert-butyl (R)-3-(2-fluoro-4-(5-methoxy-3H-[1,2,3]triazolo[4,5-b]pyridin-3-yl)-N-(8-methylisoquinolin-1-yl)benzamido)piperidine-1-carboxylate (30 mg, 0.049 mmol) in DCM (2 mL) was added 4M HCl in 1,4-dioxane (4 mL) at 0° C. and the reaction mixture was stirred at room temperature for 2 h. The reaction mixture was concentrated under reduced pressure. The residue was purified by Prep-HPLC (Conditions: Column: Luna OMEGA C-18 (250×21.2×5 μm), Mobile phase A: 0.1% formic acid in water, Mobile phase B: 100% MeCN, Gradient: Time (min)/% B: 0/45, 10/70, Flow: 15 mL/min, Dilution: MeCN+H₂O+THF). Pure fractions were combined and dried by lyophilization to afford (R)-2-fluoro-4-(5-methoxy-3H-[1,2,3]triazolo[4,5-b]pyridin-3-yl)-N-(8-methylisoquinolin-1-yl)-N-(piperidin-3-yl)benzamide formate salt (15 mg) as an off-white solid. LC-MS: [M+H⁺]=512.3. ¹H NMR (CD₃OD, 400 MHZ): δ 8.42 (d, J=5.6 Hz, 1H), 8.24 (d, J=9.2 Hz, 1H), 7.89 (dd, J=11.6 Hz, 1.6 Hz, 1H), 7.73-7.69 (m, 2H), 7.64-7.61 (m, 1H), 7.50-7.45 (m, 2H), 7.06 (t, J=8.0 Hz, 1H), 6.90 (d, J=9.2 Hz, 1H), 5.07-5.02 (m, 1H), 4.03-3.94 (m, 1H), 3.98 (s, 3H), 3.82-3.76 (m, 1H), 3.38-3.35 (m, 1H), 3.03-2.94 (m, 1H), 2.84 (s, 3H), 1.93-1.89 (m, 3H), 1.60-1.53 (m, 1H).

### Synthesis of (R)-4-(3H-[1,2,3]triazolo[4,5-b]pyridin-3-yl)-2-fluoro-N-(piperidin-3-yl)-N-(3-vinylpyridin-2-yl)benzamide (Example 188)

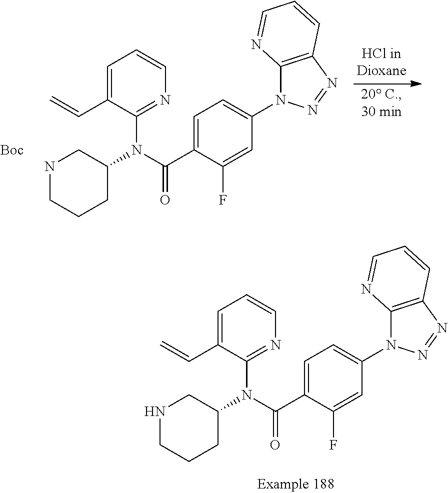 [SMILES: C=Cc1cccnc1N(C(=O)c1ccc(-n2nnc3cccnc32)cc1F)[C@@H]1CCCNC1 ; C=Cc1cccnc1N(C(=O)c1ccc(-n2nnc3cccnc32)cc1F)[C@@H]1CCCNC1 ; "Boc"]

A solution of (R)-tert-butyl 3-(4-(3H-[1,2,3]triazolo[4,5-b]pyridin-3-yl)-2-fluoro-N-(3-vinylpyridin-2-yl)benzamido)piperidine-1-carboxylate (40 mg, 68.43 μmol) in 4M HCl in dioxane (1.5 mL) was stirred at 25° C. for 30 min before it was concentrated under reduced pressure. The residue was purified by prep-HPLC (HCl condition, column: Phenomenex Luna C18 100*30 mm*3 μm; mobile phase: [H₂O (0.04% HCl)-MeCN]; gradient: 1%-30% B over 8.0 min). The pure fractions were combined and lyophilized to afford (R)-4-(3H-[1,2,3]triazolo[4,5-b]pyridin-3-yl)-2-fluoro-N-(piperidin-3-yl)-N-(3-vinylpyridin-2-yl)benzamide hydrochloride salt (10.1 mg, 33%) as a white solid. LC-MS: [M+H⁺]=444.1.

¹H NMR (400 MHZ, DMSO-d₆): δ 9.35-9.13 (m, 1H), 9.04-8.87 (m, 1H), 8.85 (br d, J=2.3 Hz, 1H), 8.67 (br d, J=8.0 Hz, 1H), 8.48 (br s, 1H), 8.20-7.89 (m, 3H), 7.62 (br dd, J=4.6, 8.4 Hz, 1H), 7.50-7.26 (m, 2H), 6.86 (br dd, J=10.8, 17.5 Hz, 1H), 5.95-5.76 (m, 1H), 5.54 (br d, J=12.3 Hz, 1H), 5.20-4.49 (m, 1H), 3.72-3.30 (m, 4H), 3.28-3.16 (m, 1H), 2.84-2.59 (m, 1H), 2.26-1.61 (m, 4H), 1.33-1.23 (m, 1H).

### Synthesis of (R)—N-(8-methylisoquinolin-1-yl)-4-phenoxy-N-(piperidin-3-yl)-1-carboxamide (Example 189)

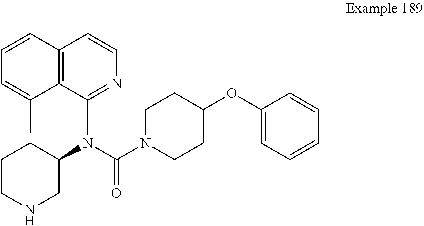 [SMILES: Cc1cccc2ccnc(N(C(=O)N3CCC(Oc4ccccc4)CC3)[C@@H]3CCCNC3)c12]

Example 189 was prepared according to the procedure described for Example 243 by substituting 3-methyl-5-(piperidin-4-yl)isoxazole with 4-phenoxypiperidine and using 4M HCl in dioxane for removal of the tert-butyloxycarbonyl group, provided the title compound as an off-white solid. LC-MS: [M+H⁺]=445.0.

### Synthesis of (R)—N-(isoquinolin-3-yl)-4-(isoxazol-5-yl)-4-methyl-N-(piperidin-3-yl)piperidine-1-carboxamide (Example 190)

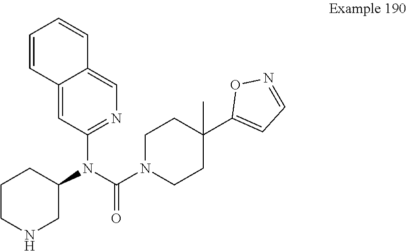 [SMILES: CC1(c2ccno2)CCN(C(=O)N(c2cc3ccccc3cn2)[C@@H]2CCCNC2)CC1]

Example 190 was prepared according to the procedure described for Example 243 by substituting tert-butyl (R)-3-((chlorocarbonyl)(isoquinolin-3-yl)amino)piperidine-1-carboxylate and 4-(1-methylpyrrolidin-3-yl)piperidine with tert-butyl (R)-3-((chlorocarbonyl)(8-methylisoquinolin-1-yl)amino)piperidine-1-carboxylate and 5-(4-methylpiperidin-4-yl)isoxazole and using 4M HCl in dioxane for removal of the tert-butyloxycarbonyl group, provided the title compound as an off-white solid. LC-MS: [M+H⁺]=420.3.

### Synthesis of (R)-4-(5-cyano-3H-[1,2,3]triazolo[4,5-b]pyridin-3-yl)-2-fluoro-N-(8-methylisoquinolin-1-yl)-N-(piperidin-3-yl)benzamide (Example 191)

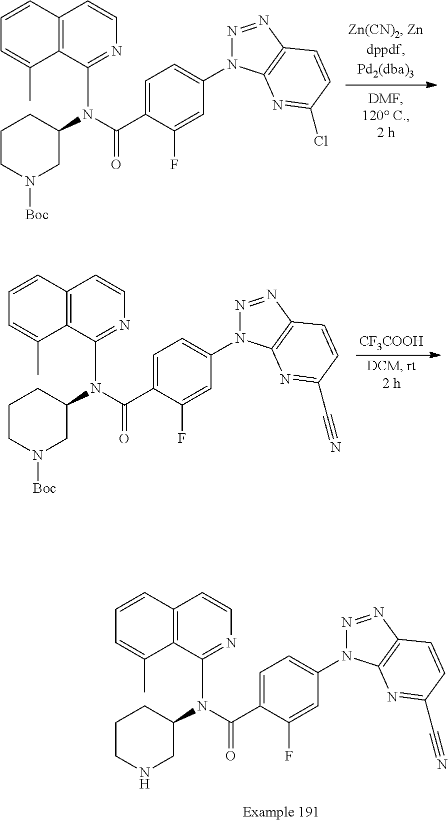 [SMILES: Cc1cccc2ccnc(N(C(=O)c3ccc(-n4nnc5ccc(Cl)nc54)cc3F)[C@@H]3CCCN(C(=O)OC(C)(C)C)C3)c12 ; Cc1cccc2ccnc(N(C(=O)c3ccc(-n4nnc5ccc(C#N)nc54)cc3F)[C@@H]3CCCN(C(=O)OC(C)(C)C)C3)c12 ; "CF3COOH" ; Cc1cccc2ccnc(N(C(=O)c3ccc(-n4nnc5ccc(C#N)nc54)cc3F)[C@@H]3CCCNC3)c12]

### Step 1:

To a solution of tert-butyl (R)-3-(4-(5-chloro-3H-[1,2,3]triazolo[4,5-b]pyridin-3-yl)-2-fluoro-N-(8-methylisoquinolin-1-yl)benzamido)piperidine-1-carboxylate (300 mg, 0.487 mmol) in DMA (10 mL) at room temperature was added Zn(CN)₂ (114.3 mg, 0.975 mmol), Zn (3.8 mg, 0.0584 mmol) and 1,1′-bis(diphenylphosphino) ferrocene (10.7 mg, 0.0194 mmol). The reaction mixture was degassed with nitrogen for 5 min and followed by addition of Pd₂(dba)₃ (17.7 mg, 0.0194 mmol). The reaction mixture was stirred at 120° C. for 5 h before it was cooled and filtered through a pad of celite. The filtrate was concentrated under reduced pressure. The residue was diluted with water (30 mL) and extracted with EtOAc (2×50 mL). The combined organic layer was dried over Na₂SO₄ and concentrated under reduced pressure. The residue was purified by Combi-flash using 80% EtOAc in hexane to afford tert-butyl (R)-3-(4-(5-cyano-3H-[1,2,3]triazolo[4,5-b]pyridin-3-yl)-2-fluoro-N-(8-methylisoquinolin-1-yl)benzamido)piperidine-1-carboxylate (130 mg) as an off-white solid. LC-MS: [M+H⁺]=607.30.

### Step 2:

To a solution of tert-butyl (R)-3-(4-(5-cyano-3H-[1,2,3]triazolo[4,5-b]pyridin-3-yl)-2-fluoro-N-(8-methylisoquinolin-1-yl)benzamido)piperidine-1-carboxylate (50 mg, 0.082 mmol) in DCM (5 ml) at 0° C. was added TFA (2 mL). The reaction mixture was stirred at room temperature for 2 h. The reaction mixture was concentrated under reduced pressure to afford (R)-4-(5-cyano-3H-[1,2,3]triazolo[4,5-b]pyridin-3-yl)-2-fluoro-N-(8-methylisoquinolin-1-yl)-N-(piperidin-3-yl)benzamide TFA salt (40 mg) as a white-solid. The residue was purified by Prep-HPLC (Conditions: Luna OMEGA C-18 (250×21.2×5 μm), Mobile phase A: 0.1% formic acid in water (100%), Mobile phase B: 100% MeCN, Gradient: Time (min)/% B: 0/10, 2/50, 10/50, Flow: 15 mL/min, Dilution: MeCN+H₂O+THF). Pure fractions were dried by lyophilization to afford (R)-4-(5-cyano-3H-[1,2,3]triazolo[4,5-b]pyridin-3-yl)-2-fluoro-N-(8-methylisoquinolin-1-yl)-N-(piperidin-3-yl)benzamide trifluoroacetic acid salt (35 mg) as an off-white solid. LC-MS: [M+H⁺]=507.80. ¹H NMR (CD₃OD, 400 MHZ): δ 8.92-8.90 (m, 1H), 8.64-8.63 (m, 1H), 8.1-8.12 (m, 1H), 8.08-8.06 (m, 1H), 7.97-7.71 (m, 2H), 7.83-7.80 (m, 1H), 7.69-7.67 (m, 2H), 7.35-7.31 (m, 1H), 5.24-5.11 (m, 1H), 4.19-4.16 (m, 1H), 3.98-3.92 (m, 1H), 3.15-3.08 (m, 1H), 3.05 (s, 3H), 2.20-2.10 (m, 2H), 1.81-1.71 (m, 1H), 1.41-1.41 (m, 1H), 1.12-1.01 (m, 1H).

### Synthesis of (R)-4-(5-chloro-3H-[1,2,3]triazolo[4,5-b]pyridin-3-yl)-2-fluoro-N-(8-methylisoquinolin-1-yl)-N-(piperidin-3-yl)benzamide (Example 192)

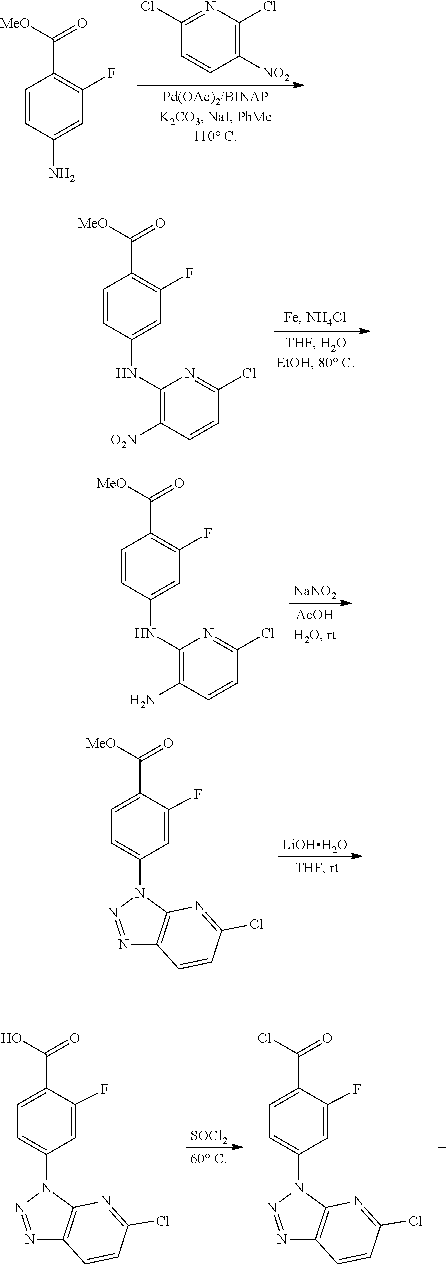

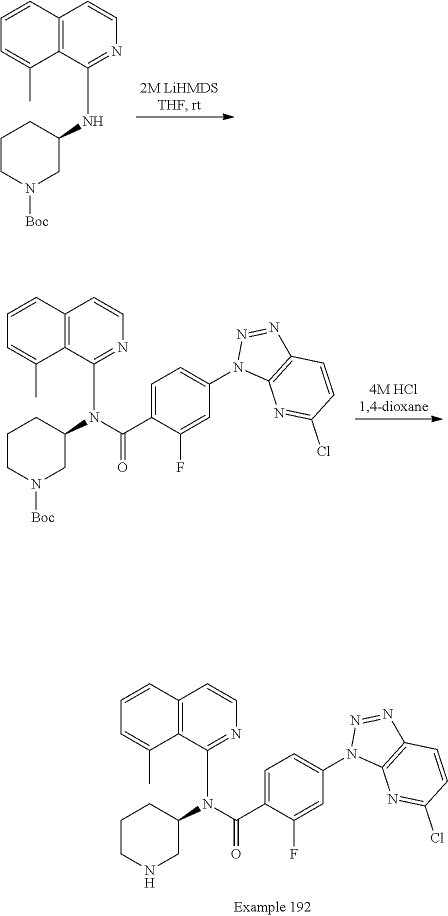 [SMILES: Cc1cccc2ccnc(N[C@@H]3CCCN(C(=O)OC(C)(C)C)C3)c12 ; Cc1cccc2ccnc(N(C(=O)c3ccc(-n4nnc5ccc(Cl)nc54)cc3F)[C@@H]3CCCN(C(=O)OC(C)(C)C)C3)c12 ; Cc1cccc2ccnc(N(C(=O)c3ccc(-n4nnc5ccc(Cl)nc54)cc3F)[C@@H]3CCCNC3)c12]

### Step 1:

To a solution of methyl 4-amino-2-fluorobenzoate (4 g, 23.64 mmol) and 2,6-dichloro-3-nitropyridine (5.4 g, 28.3 mmol) in Toluene (50 mL) at room temperature was added NaI (1.6 g, 0.70 mmol), K₂CO₃ (6.5 g, 47.28 mmol) and BINAP (294.3 mg, 0.47 mmol). The reaction mixture was degassed with nitrogen for 15 min and followed by addition of palladium acetate (105.5 mg, 0.47 mmol). The reaction mixture and stirred at 110° C. for 16 h before it was allowed to cool. The reaction mixture was diluted with EtOAc (150 mL), and washed with water (100 mL). The organic layer was dried over Na₂SO₄ and concentrated under reduced pressure. The residue was purified by Combi-flash using 80% EtOAc in hexane to afford methyl 4-((6-chloro-3-nitropyridin-2-yl)amino)-2-fluorobenzoate (1.8 g) as a yellow solid. LC-MS: [M+H⁺-Boc]=326.05.

### Step 2:

To a solution of methyl 4-((6-chloro-3-nitropyridin-2-yl)amino)-2-fluorobenzoate (1.8 g, 5.53 mmol) in MeOH (10 mL), THF (5 mL), and water (5 mL) at room temperature was added iron powder (1.5 g, 27.68 mmol) followed by NH₄Cl (2.3 g, 44.30 mmol) in one portion. The reaction mixture was then stirred at 80° C. for 3 h. The reaction mixture was filtered through a pad of celite, and the filtrate was concentrated under reduced pressure. The residue was diluted with water, and extracted with EtOAc (2×250 mL). The combined organic layer was washed with brine, dried over Na₂SO₄, and concentrated under reduced pressure to afford methyl 4-((3-amino-6-chloropyridin-2-yl)amino)-2-fluorobenzoate (1.2 g) as a brown solid. LC-MS: [M+H⁺-boc]=296.

### Step 3:

To a solution of methyl 4-((3-amino-6-chloropyridin-2-yl)amino)-2-fluorobenzoate (1.2 g, 4.06 mmol) in acetic acid (10 mL) and water (5 mL) at 0° C. was added sodium nitrite (364 mg, 5.28 mmol) and the reaction mixture was stirred at room temperature for 2 h. The reaction mixture was diluted with water and extracted with DCM (2×50 mL). The combined organic layer was washed with brine, dried over Na₂SO₄, and concentrated under reduced pressure to afford methyl 4-(5-chloro-3H-[1,2,3]triazolo[4,5-b]pyridin-3-yl)-2-fluorobenzoate (1.1 g) as a brown solid. LC-MS: [M+H⁺-boc]=307.1.

### Step 4:

To a solution of methyl 4-(5-chloro-3H-[1,2,3]triazolo[4,5-b]pyridin-3-yl)-2-fluorobenzoate (550 mg, 1.79 mmol) in THF (10 mL) at 0° C. was added LiOH·H₂O (215 mg, 8.98 mmol) and H₂O (10 mL) and then stirred at room temperature for 2 h. The reaction mixture was concentrated under reduced pressure, diluted with water, and acidified to pH 2 with dilute HCl. The precipitate was isolated by filtration and dried to afford 4-(5-chloro-3H-[1,2,3]triazolo[4,5-b]pyridin-3-yl)-2-fluorobenzoic acid (410 mg) as an off-white solid. LC-MS: [M+H⁺-boc]=293.

### Step 5:

To a solution of 4-(5-chloro-3H-[1,2,3]triazolo[4,5-b]pyridin-3-yl)-2-fluorobenzoic acid (410 mg, 1.45 mmol) in SOCl₂ (10 mL) at 0° C. was added a catalytic amount of DMF (0.1 mL) and the reaction mixture was stirred at 90° C. for 16 h. It was then concentrated under reduced pressure and co-distilled with toluene to afford 4-(5-chloro-3H-[1,2,3]triazolo[4,5-b]pyridin-3-yl)-2-fluorobenzoyl chloride (500 mg) as an off-yellow solid.

### Step 6:

To a solution of tert-butyl (R)-3-((8-methylisoquinolin-1-yl)amino)piperidine-1-carboxylate (200 mg, 0.586 mmol) in THF (10 mL) was added 1.5M LiHMDS in THF (1.1 mL, 1.75 mmol) at 0° C. The reaction mixture was stirred for 10 min and followed by addition of 4-(5-chloro-3H-[1,2,3]triazolo[4,5-b]pyridin-3-yl)-2-fluorobenzoyl chloride (400 mg, 1.28 mmol) in THF (3 mL). The reaction was stirred for 30 min before it was quenched with ice cold water and extracted with EtOAc (2×50 mL). The combined organic layer was washed with brine, dried over Na₂SO₄, and concentrated under reduced pressure. The residue was purified by combi-flash using 80% EtOAc in hexane to afford an impure product which was further purified by Prep-HPLC (Conditions: Column: X-SELECT CSH C18 (250*19) mm, 5 μm, Mobile phase A: 0.1% formic acid in water, Mobile phase B: 100% MeCN, Gradient: Time (min)/% B: 0/45, 10/75, Flow: 15 mL/min, Dilution: MeCN+H₂O+THF). Pure fractions were combined and dried by lyophilization to afford tert-butyl (R)-3-(4-(5-chloro-3H-[1,2,3]triazolo[4,5-b]pyridin-3-yl)-2-fluoro-N-(8-methylisoquinolin-1-yl)benzamido)piperidine-1-carboxylate (120 mg) as an off-white solid. LC-MS: [M+H⁺]=616.

### Step 7:

The title compound hydrochloride salt was obtained as a white solid by deprotection Method A used in Example 97. LC-MS: [M+H⁺]=464. ¹H NMR (Methanol-d4, 400 MHZ) δ 8.47-8.40 (m, 2H), 7.87-7.80 (m, 1H), 7.73-7.69 (m, 2H), 7.63-7.61 (m, 1H), 7.53-7.51 (m, 1H), 7.48 (d, J=4.8 Hz, 2H), 7.10-7.06 (m, 1H), 5.05-5.02 (m, 1H), 4.01-3.99 (m, 1H), 3.81 (t, J=11.6 Hz, 1H), 3.44-3.39 (m, 2H), 3.10-2.98 (m, 2H), 2.84 (s, 3H), 1.95-1.89 (m, 2H).

### Synthesis of (R)-2-fluoro-4-(1-methyl-1H-1,2,3-triazol-4-yl)-N-(8-methylisoquinolin-1-yl)-N-(piperidin-3-yl)benzamide (Example 193)

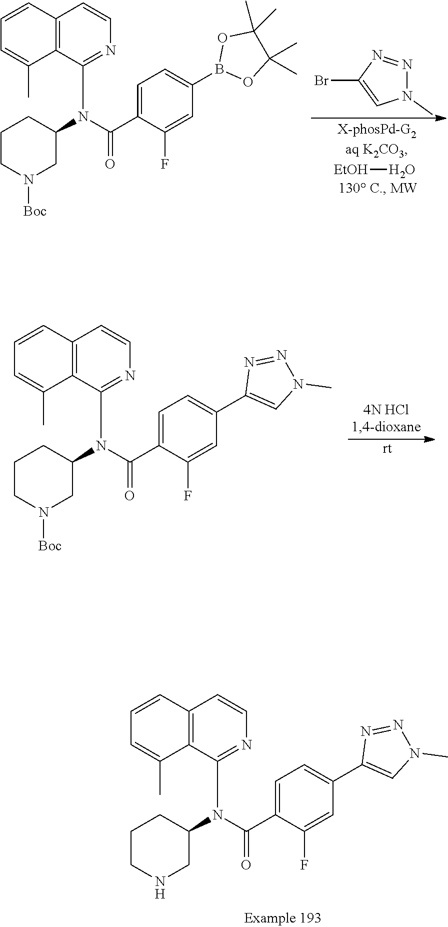

Example 193 was prepared according to the procedure described for Example 127 by substituting 2-bromo-5-methyl-1,3,4-thiadiazole with 4-bromo-1-methyl-1H-1,2,3-triazole, provided the title compound hydrochloride salt as a white solid. LC-MS: [M+H⁺]=445.20. ¹H NMR (CD₃OD, 400 MHZ) δ 8.40-8.36 (m, 1H), 8.13 (s, 1H), 7.70-7.65 (m, 1H), 7.61-7.56 (m, 1H), 7.48-7.40 (m, 2H), 7.22-7.15 (m, 1H), 7.09 (d, J=8.0 Hz, 1H), 6.84-6.79 (m, 1H), 5.06-4.99 (m, 1H), 4.04 (s, 3H), 3.98-3.95 (m, 1H), 3.81-3.76 (m, 1H), 3.38-3.35 (m, 1H), 3.00-2.96 (m, 1H), 2.80 (s, 3H), 2.69-2.66 (m, 1H), 1.93-1.91 (m, 2H), 1.59-1.52 (m, 1H).

### Synthesis of (R)-2-fluoro-N-(8-methylisoquinolin-1-yl)-N-(piperidin-3-yl)-4-(2H-1,2,3-triazol-2-yl)benzamide (Example 194)

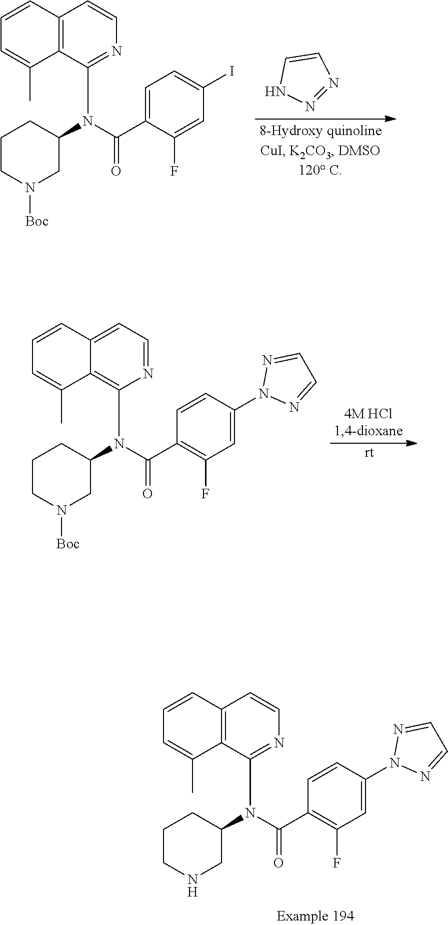 [SMILES: Cc1cccc2ccnc(N(C(=O)c3ccc(I)cc3F)[C@@H]3CCCN(C(=O)OC(C)(C)C)C3)c12 ; c1c[nH]nn1 ; Cc1cccc2ccnc(N(C(=O)c3ccc(-n4nccn4)cc3F)[C@@H]3CCCN(C(=O)OC(C)(C)C)C3)c12 ; Cc1cccc2ccnc(N(C(=O)c3ccc(-n4nccn4)cc3F)[C@@H]3CCCNC3)c12]

### Step 1

To a mixture of tert-butyl (R)-3-(2-fluoro-4-iodo-N-(8-methylisoquinolin-1-yl)benzamido)piperidine-1-carboxylate (200 mg, 0.339 mmol) and 1H-1,2,3-triazole (28 mg, 0.405 mmol) in anhydrous DMSO (2 mL) at room temperature was added K₂CO₃ (50 mg, 0.361 mmol) and 8-hydroxy quinoline (9 mg, 0.062 mmol). The reaction mixture was degassed with nitrogen and followed by addition of CuI (32 mg, 0.168 mmol). The reaction mixture was stirred at 120° C. for 1 h before it was cooled. The reaction mixture was diluted with water (30 mL) and extracted with EtOAc (2×30 mL). The combined organic layer was dried over Na₂SO₄, concentrated under reduced pressure. The crude product was purified by Combi-flash using 10-80% EtOAc in hexane, and then further purified by Prep-HPLC (Conditions: Column: X-SELECT CSH C18 (250*19) mm, 5 μm, Mobile phase A: 0.1% formic acid in water, Mobile phase B: 100% MeCN, Gradient: Time (min)/% B: 0/40, 10/80, Flow: 15 mL/min, Dilution: MeCN+H₂O+THF). Pure fractions were combined and dried by lyophilization to afford tert-butyl (R)-3-(2-fluoro-N-(8-methylisoquinolin-1-yl)-4-(2H-1,2,3-triazol-2-yl)benzamido)piperidine-1-carboxylate (15 mg) as an off-white solid. LC-MS: [M+H⁺]=531.6.

### Step 2:

The title compound hydrochloride salt was obtained as an off-white solid by deprotection Method A used in Example 97. LC-MS: [M+H⁺]=431.3. ¹H NMR (DMSO-d₆, 400 MHZ): δ 8.53-8.41 (m, 1H), 8.09 (s, 2H), 7.84-7.80 (m, 1H), 7.74-7.69 (m, 1H), 7.57-7.49 (m, 3H), 7.40-7.37 (m, 1H), 7.01-6.96 (m, 1H), 4.97-4.93 (m, 1H), 3.83-3.80 (m, 1H), 3.56-3.46 (m, 1H), 3.28-3.25 (m, 1H), 2.95-2.80 (m, 1H), 2.77 (s, 3H), 1.82-1.76 (m, 2H), 1.36-1.18 (m, 1H), 0.89-0.85 (m, 1H).

### Synthesis of (R)-2-fluoro-4-(5-methyl-2H-tetrazol-2-yl)-N-(8-methylisoquinolin-1-yl)-N-(piperidin-3-yl) benzamide (Example 195)

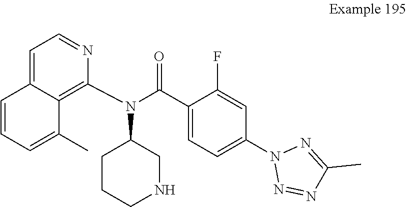 [SMILES: Cc1nnn(-c2ccc(C(=O)N(c3nccc4cccc(C)c34)[C@@H]3CCCNC3)c(F)c2)n1]

Example 195 was prepared according to the procedure described for Example 142 by substituting propionimidamide with acetimidamide, provided the title compound hydrochloride salt as an off-white solid. LC-MS: [M+H⁺]=LC-MS: [M+H⁺]=447.0. ¹H NMR (CD₃OD, 400 MHZ): δ 8.35-8.31 (m, 1H), 7.65-7.61 (m, 1H), 7.56-7.52 (m, 1H), 7.48-7.35 (m, 4H), 7.00-6.95 (m, 1H), 5.00-4.94 (m, 1H), 3.93-3.90 (m, 1H), 3.75-3.72 (m, 1H), 3.35-3.29 (m, 1H), 2.94-2.91 (m, 1H), 2.75 (s, 3H), 2.41 (s, 3H), 1.86-1.47 (m, 3H), 1.29-1.18 (m, 1H).

### Synthesis of (R)-4-(benzo[c]isoxazol-3-yl)-2-fluoro-N-(8-methylisoquinolin-1-yl)-N-(piperidin-3-yl)benzamide (Example 196)

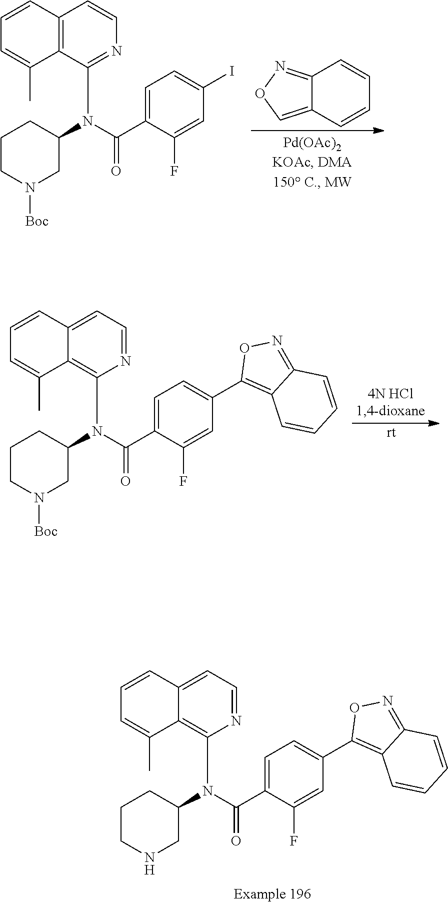

### Step 1:

To a solution of tert-butyl (R)-3-(2-fluoro-4-iodo-N-(8-methylisoquinolin-1-yl)benzamido)piperidine-1-carboxylate (200 mg, 0.33 mmol) and benzo[c]isoxazole (44 mg, 0.37 mmol) in DMA (3 mL) at room temperature was added KOAc (100 mg, 1.01 mmol) and Pd(OAc)₂ (7.6 mg, 0.033 mmol). The reaction mixture was irradiated in the microwave at 150° C. for 1 h. The reaction mixture was diluted with water and extracted with EtOAc. The organic layer was washed with brine, dried over Na₂SO₄, and concentrated under reduced pressure. The residue was purified by combi-flash using 50% EtOAc in hexane and then was further purified by Prep-HPLC (Conditions: Column: SUNFIRE OBD PREP C-18 (250×21.2×5 μm), Mobile phase A: 0.1% formic acid in water, Mobile phase B: 100% MeCN, Gradient: Time (min)/% B: 0/85, 10/95. Flow: 15 mL/min, Dilution: MeCN+H₂O+THF). Pure fractions were combined and dried by lyophilization to afford tert-butyl (R)-3-(4-(benzo[c]isoxazol-3-yl)-2-fluoro-N-(8-methylisoquinolin-1-yl)benzamido)piperidine-1-carboxylate (150 mg) as a yellow solid.

### Step 2:

To a solution of tert-butyl (R)-3-(4-(benzo[c]isoxazol-3-yl)-2-fluoro-N-(8-methylisoquinolin-1-yl)benzamido)piperidine-1-carboxylate (150 mg, 0.25 mol) in 1,4-dioxane (1 mL) was added 4M HCl in 1,4-dioxane (2 mL) at 0° C. and the reaction mixture was stirred at room temperature for 1 h. The reaction mixture was concentrated under reduced pressure and the residue was purified by Prep-HPLC (Conditions: Column: Luna OMEGA C-18 (250×21.2×5 μm), Mobile phase A: 0.1% formic acid in water, Mobile phase B: 100% MeCN, Gradient: Time (min)/% B: 0/10, 2/10, 10/50. Flow: 15 mL/min, Dilution: MeCN+H₂O+THF). Pure fractions were combined and dried by lyophilization to afford (R)-4-(benzo[c]isoxazol-3-yl)-2-fluoro-N-(8-methylisoquinolin-1-yl)-N-(piperidin-3-yl)benzamide formic acid salt (30 mg) as a yellow solid. LC-MS: [M+H⁺]=480.8. ¹H NMR (CD₃OD, 400 MHZ): δ 8.41 (bs, 1H), 8.34 (d, J=5.6 Hz, 1H), 7.68-7.60 (m, 2H), 7.55-7.52 (m, 1H), 7.47-7.38 (m, 4H), 7.35-7.27 (m, 2H), 7.05-7.02 (m, 1H), 6.99-6.95 (m, 1H), 4.97-4.91 (m, 1H), 3.91 (dd, J=13.6 Hz, 2.0 Hz, 1H), 3.69 (t, J=10.8 Hz, 1H), 3.29-3.25 (m, 1H), 2.90-2.83 (m, 1H), 2.80 (s, 3H), 1.85-1.83 (m, 3H), 1.53-1.46 (m, 1H).

### Synthesis of (R)—N-(8-methylisoquinolin-1-yl)-4-(3-methylisothiazol-5-yl)-N-(piperidin-3-yl)piperidine-1-carboxamide (Example 197)

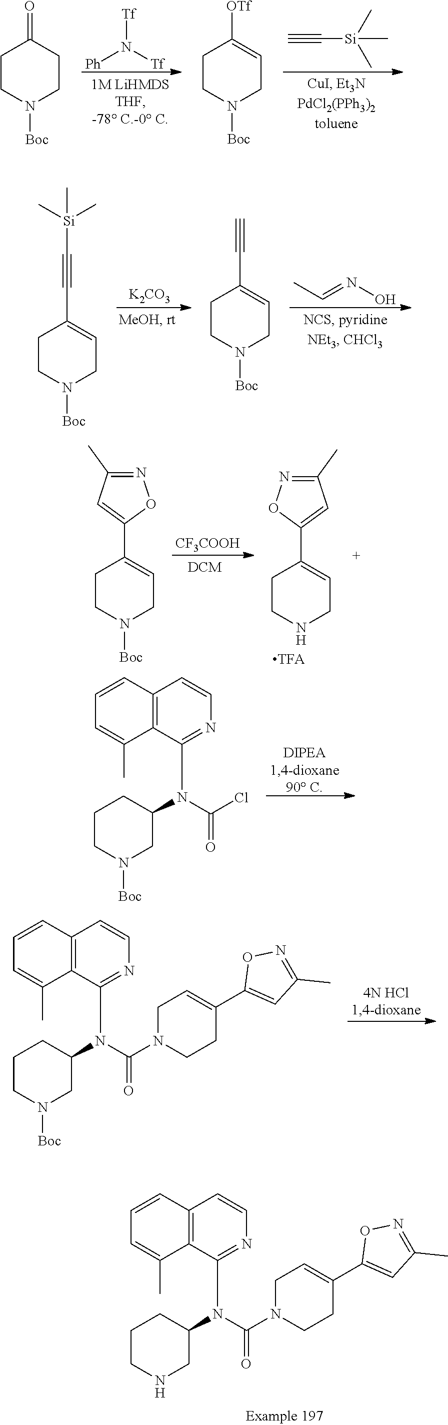

### Step 1:

To a solution of tert-butyl 4-oxopiperidine-1-carboxylate (5 g, 25.09 mmol) in THF (30 mL) at −78° C. was added 1M LiHMDS in THF (30.11 mL, 30.11 mmol). The solution was then stirred for 30 min at −78° C. and followed by addition of 1,1,1-trifluoro-N-phenyl-N-((trifluoromethyl) sulfonyl) methanesulfonamide (9.86 g, 27.60 mmol) in THF (30 mL). The reaction was warmed to 0° C. and stirred at for 0° C. 1 h. The reaction mixture was diluted with a saturated NaHCO₃ solution (60 mL), and extracted with EtOAc (2×80 mL). The combined organic layer was washed with brine (60 mL), dried over Na₂SO₄, and concentrated. The residue was purified by Combi-flash using 10% EtOAc in hexane to afford crude tert-butyl 4-(((trifluoromethyl)sulfonyl) oxy)-3,6-dihydropyridine-1(2H)-carboxylate (6 g) as a colorless liquid.

### Step 2:

To a solution of tert-butyl 4-(((trifluoromethyl)sulfonyl)oxy)-3,6-dihydropyridine-1 (2H)-carboxylate (6 g, 18.11 mmol) in toluene (35 mL) at room temperature were added CuI (34 mg, 0.18 mmol) and TEA (5.23 mL, 36.23 mmol). The reaction mixture was degassed with nitrogen for 5 min and followed by addition of Pd(PPh₃)₂Cl₂ (636 mg, 0.90 mmol) and trimethylsilylacetylene (7.7 mL, 54.34 mmol). The reaction mixture was stirred for 3 h, after which it was concentrated under reduced pressure. The residue was purified by Combi-flash using 4% EtOAc in hexane to afford crude tert-butyl 4-((trimethylsilyl)ethynyl)-3,6-dihydropyridine-1(2H)-carboxylate (2.5 g) as a yellowish liquid.

### Step 3:

To a solution of tert-butyl 4-((trimethylsilyl) ethynyl)-3,6-dihydropyridine-1(2H)-carboxylate (2.5 g, 8.95 mmol) in MeOH (30 mL) was added K₂CO₃ (3.71 g, 26.86 mmol). The reaction mixture was stirred for 3 h, after which it was diluted with water (60 mL) and extracted with ethyl acetate (2×60 mL). The combined organic layer was washed with brine, dried over Na₂SO₄, and concentrated under reduced pressure. The residue was purified by Combi-flash using 2% EtOAc in hexane to afford crude tert-butyl 4-ethynyl-3,6-dihydropyridine-1(2H)-carboxylate (800 mg) as a syrup.

### Step 4:

To a solution of acetaldehyde oxime (235 mg, 3.98 mmol) in CHCl₃ (8 mL) were added pyridine (1.5 mL) and NCS (531 mg, 3.98 mmol). The reaction was stirred at room temperature for 1 h and followed by addition of tertbutyl 4-ethynyl-3,6-dihydropyridine-1 (2H)-carboxylate (750 mg, 3.62 mmol) in CHCl₃ (7 mL) and dropwise addition of triethylamine (0.63 mL, 4.52 mmol). The reaction was stirred for 16 h before it was concentrated under reduced pressure. The residue was purified by Combi-flash using 20% EtOAc in hexane to afford crude tert-butyl 4-(3-methylisoxazol-5-yl)-3,6-dihydropyridine-1 (2H)-carboxylate (160 mg) as a gummy solid. LC-MS: [M+H⁺]=265.15.

### Step 4:

To a solution of tert-butyl 4-(3-methylisoxazol-5-yl)-3,6-dihydropyridine-1(2H)-carboxylate (180 mg, 0.68 mmol) in DCM (6 mL) at 0° C. was added TFA (3 mL). The reaction mixture was allowed to warm to room temperature and stir for 3 h. The reaction mixture was concentrated under reduced pressure to afford 3-methyl-5-(1,2,3,6-tetrahydropyridin-4-yl) isoxazole TFA salt (90 mg, 0.15 mmol, 52%) as yellow solid. LC-MS: [M+H⁺]=165.1.

### Step 5:

To a solution of 3-methyl-5-(1,2,3,6-tetrahydropyridin-4-yl)isoxazole TFA salt (134 mg, 0.48 mmol) in 1,4-dioxane (10 mL) were added DIPEA (0.41 mL, 2.23 mmol) and tert-butyl (R)-3-((chlorocarbonyl)(8-methylisoquinolin-1-yl)amino)piperidine-1-carboxylate (150 mg, 0.37 mmol). The reaction mixture was then warmed to 90° C. and stirred for 3 h. The reaction mixture was then diluted with water (30 mL), and extracted with ethyl acetate (2×60 mL). The combined organic layer was washed with a saturated brine solution, dried over Na₂SO₄, and concentrated under reduced pressure. The residue was purified by Combi-flash using 33% EtOAc in hexane first, and then further purified by Prep-HPLC (Conditions: Column: Luna OMEGA C-18 (250×21.2×5 μm), Mobile phase A: 0.1% formic acid in water, Mobile phase B: 100% MeCN, Gradient: T/% B: 0/65, 10/95. Flow: 15 mL/min, Dilution: MeCN+H₂O+THF). The product was lyophilized to afford (R)-3-(N-(8-methylisoquinolin-1-yl)-4-(3-methylisoxazol-5-yl)-1,2,3,6-tetrahydropyridine-1-carboxamido)piperidine-1-carboxylate (60 mg, 30%) as a white solid. LC-MS: [M+H⁺]=532.3.

### Step 6:

The title compound hydrochloride salt was obtained as an off-white solid by deprotection Method A used in Example 97. LC-MS: [M+H⁺]=432.2. ¹H NMR (CD₃OD, 400 MHZ): δ 8.32 (d, J=5.2 Hz, 1H), 7.80-7.71 (m, 2H), 7.62-7.52 (m, 1H), 7.41 (d, J=6.8 Hz, 1H), 6.03 (m, 1H), 5.92 (s, 1H), 4.78-4.71 (m, 1H), 3.80-3.71 (m, 1H), 3.63-3.55 (m, 2H), 3.30-3.24 (m, 2H), 3.15-3.10 (m, 2H), 2.85-2.79 (m, 3H), 2.63 (s, 3H), 2.09 (s, 3H), 1.86 (m, 1H), 1.78-1.71 (m, 3H).

### Synthesis of (R)-2-fluoro-N-(8-methylisoquinolin-1-yl)-4-(6-cyanopyrazin-2-yl)-N-(piperidin-3-yl)benzamide (Example 198)

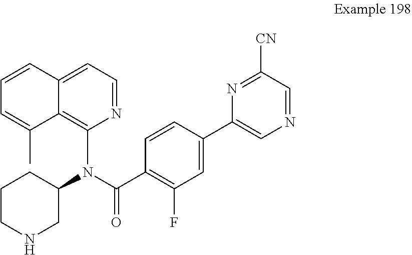 [SMILES: Cc1cccc2ccnc(N(C(=O)c3ccc(-c4cncc(C#N)n4)cc3F)[C@@H]3CCCNC3)c12]

Example 198 was prepared according to the procedure described for Example 203 by substituting 2-bromo-3-methylpyrazine with 2-bromo-6-cyanopyrazine, provided the title compound hydrochloride salt as an off-white solid. LC-MS: [M+H⁺]=467.45. ¹H NMR (CD₃OD, 400 MHZ): δ 9.07 (s, 1H), 8.81 (s, 1H), 8.32-8.29 (m, 1H), 7.61-7.57 (m, 1H), 7.54-7.48 (m, 2H), 7.42-7.39 (m, 3H), 6.96-6.91 (m, 1H), 4.94-4.86 (m, 1H), 3.83-3.80 (m, 1H), 3.56-3.50 (m, 1H), 3.16-3.12 (m, 1H), 2.76 (s, 3H), 2.72-2.60 (m, 1H), 1.82-1.71 (m, 3H), 1.39-1.36 (m, 1H).

### Synthesis of (R)—N-(8-methylisoquinolin-1-yl)-4-(2-methylpyridin-4-yl)-N-(piperidin-3-yl)piperidine-1-carboxamide (Example 199)

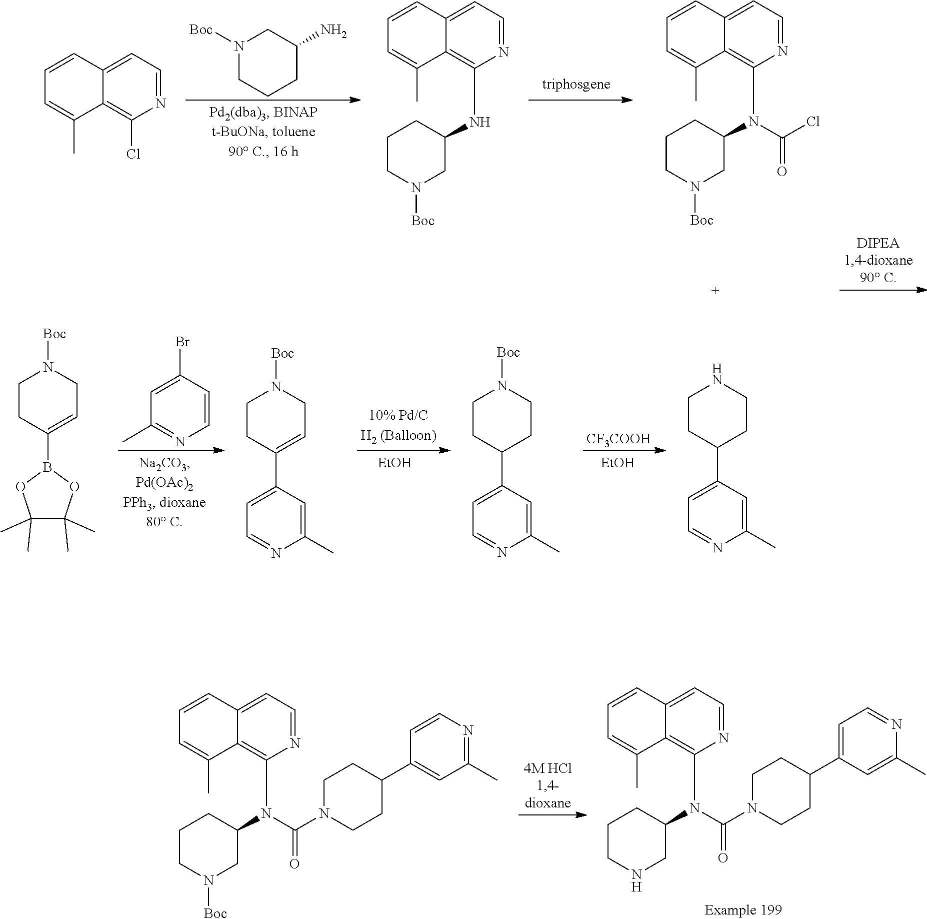

### Step 1:

1-chloro-8-methylisoquinoline (10 g, 56.30 mmol), (R)-tert-butyl 3-aminopiperidine-1-carboxylate (11.21 g, 56.30 mmol), t-BuONa (10.82 g, 112.59 mmol), BINAP (3.51 g, 5.63 mmol) and Pd₂(dba)₃ (5.16 g, 5.63 mmol) in toluene (150 mL) was degassed with nitrogen for 2 min and then heated to 90° C. for 16 h under nitrogen atmosphere. The reaction mixture was poured into H₂O (100 mL) and extracted with ethyl acetate (3×150 mL). The combined organic phase was washed with brine (100 mL), dried over anhydrous Na₂SO₄, and concentrated under reduced pressure. The crude residue was purified by flash silica gel chromatography (30% ethyl acetate in petroleum ether) to give tert-butyl (R)-3-((8-methylisoquinolin-1-yl)amino)piperidine-1-carboxylate (14 g, 72.83%) as a yellow oil.

### Step 2:

To a solution of tert-butyl (R)-3-((8-methylisoquinolin-1-yl)amino)piperidine-1-carboxylate (8.00 g, 23.42 mmol) in THF (150 mL) at −65° C. was added TEA (9.78 mL, 90.29 mmol) and triphosgene (20.86 g, 70.29 mmol). Then the mixture was allowed to slowly warm to 25° C. and then stirred at 25° C. for 18 h. The reaction mixture was filtered, and the filtrate was quenched with saturated aq. NH₄Cl (60 mL). The filtrate was extracted with ethyl acetate (3×100 mL), and the combined organic layer was dried over MgSO₄, filtered, and concentrated under reduced pressure. The residue was purified by flash silica gel chromatography (30% ethyl acetate in petroleum ether) to give tert-butyl (R)-3-((chlorocarbonyl)(8-methylisoquinolin-1-yl)amino)piperidine-1-carboxylate (7.4 g, 78%) as a yellow oil.

### Step 3:

To a mixture of tert-butyl 4-(4,4,5,5-tetramethyl-1,3,2-dioxaborolan-2-yl)-3,6-dihydropyridine-1(2H)-carboxylate (1.0 g, 3.23 mmol) and 4-bromo-2-methylpyridine (827 mg, 4.85 mmol) in 1,4-dioxane:H₂O (15 mL) at room temperature was added Pd(dppf)₂Cl₂·DCM (52.7 mg, 0.064 mmol) and Cs₂CO₃ (210 mg, 0.646 mmol). The reaction mixture was then heated and stirred at 110° C. for 16 h. The reaction was cooled to 25° C. before being diluted with water (20 mL) and extracted with ethyl acetate (3×50 mL). The combined organic layer was washed with brine, dried over Na₂SO₄, and concentrated under reduced pressure. The crude residue was purified by Combi-flash using 40% EtOAc in hexane to afford tert-butyl 2′-methyl-3,6-dihydro-[4,4′-bipyridine]-1(2H)-carboxylate (800 mg, 89%) as an off-white oil. LC-MS: [M+H⁺]=275.

### Step 4:

To a solution of tert-butyl 2′-methyl-3,6-dihydro-[4,4′-bipyridine]-1(2H)-carboxylate (400 mg, 1.45 mmol) in EtOH (5 mL) at room temperature was added Pd/C (10 wt %, 50 mg). The reaction was stirred under H₂ (balloon pressure) for 16 h. The reaction mixture was then filtered through a pad of celite. The filtrate was concentrated under reduced pressure to afford tert-butyl 4-(2-methylpyridin-4-yl)piperidine-1-carboxylate (400 mg, 62%) as a light brown oil.

### Step 5:

To a solution of tert-butyl 4-(2-methylpyridin-4-yl)piperidine-1-carboxylate (400 mg, 1.44 mmol) in DCM (1.0 mL) at 0° C. was added TFA (0.330 mL, 4.32 mmol). The reaction mixture was warmed to room temperature and stirred for 3 h. The reaction mixture was quenched with saturated aq. NaHCO₃ (50 mL) and extracted with 10% MeOH in DCM (2×100 mL). The combined organic layer was dried over Na₂SO₄ and concentrated under reduced pressure to afford crude 2-methyl-4-(piperidin-4-yl)pyridine trifluoroacetic acid salt (250 mg) as an off-white solid. LC-MS: [M+H⁺]=177.1.

### Step 6:

To a stirred solution of tert-butyl (R)-3-((chlorocarbonyl)(8-methylisoquinolin-1-yl)amino)piperidine-1-carboxylate (150 mg, 0.37 mmol) in 1,4-dioxane (2.5 mL) at room temperature were added DIPEA (0.199 mL, 1.11 mmol) and 2-methyl-4-(piperidin-4-yl)pyridine TFA salt (202 mg, 0.74 mmol). The reaction mixture was heated to 100° C. and stirred for 16 h. The reaction mixture was then diluted with water (20 mL) and extracted with ethyl acetate (2×40 mL). The combined organic layer was dried over Na₂SO₄ and concentrated under reduced pressure. The residue was purified by Combi-flash using 40% EtOAc in hexane to afford tert-butyl (R)-3-(N-(8-methylisoquinolin-1-yl)-4-(2-methylpyridin-4-yl)piperidine-1-carboxamido)-piperidine-1-carboxylate (90 mg, 45%) as a yellowish oil. LC-MS: [M-Boc+H⁺]=444.25.

### Step 7:

The title compound hydrochloride salt was obtained as an off-white solid by deprotection Method A used in Example 97. LC-MS: [M+H⁺]=444.25. ¹H NMR (CD₃OD, 400 MHZ) δ 8.53-8.50 (m, 1H), 8.42-8.40 (m, 1H), 7.92-7.86 (m, 2H), 7.79-7.76 (m, 1H), 7.68-7.60 (m, 1H), 7.45 (bs, 1H), 7.39 (d, J=5.6 Hz, 1H), 4.83-4.79 (m, 1H), 3.98-3.92 (m, 1H), 3.73-3.65 (m, 2H), 3.37-3.21 (m, 2H), 2.98-2.93 (m, 1H), 2.79 (s, 3H), 2.70 (s, 3H), 2.80-2.70 (m, 2H), 2.61-2.51 (m, 2H), 2.01-1.87 (m, 3H), 1.68-1.65 (m, 1H), 1.38-1.10 (m, 3H).

### Synthesis of (R)-5-(3H-[1,2,3]triazolo[4,5-b]pyridin-3-yl)-N-(8-chloroisoquinolin-1-yl)-3-fluoro-N-(piperidin-3-yl)picolinamide (Example 200)

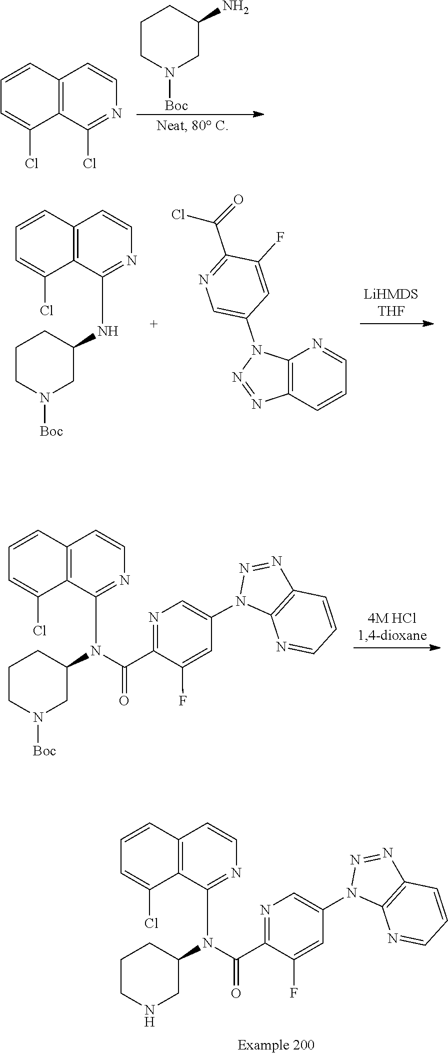

### Step 1:

1,8-dichloroisoquinoline (2.5 g, 12.623 mmol) and tert-butyl (R)-3-aminopiperidine-1-carboxylate (5 g, 25.246 mmol) were placed in a sealed tube and stirred at 80° C. for 5 h. The reaction mixture was quenched with ice cold water and extracted with EtOAc (2×50 mL). The combined organic layer was washed with brine, dried over Na₂SO₄, and concentrated under reduced pressure. The residue was purified by Combi-flash, eluting with 30% EtOAc in hexane to affords tert-butyl (R)-3-((8-chloroisoquinolin-1-yl)amino)piperidine-1-carboxylate (3 g, 66%) as pale-green gummy solid. LC-MS: [M+H⁺]=361.8.

### Step 2:

To a solution of tert-butyl (R)-3-((8-chloroisoquinolin-1-yl)amino)piperidine-1-carboxylate (200 mg, 0.552 mmol) in THF (10 mL) at 0° C. was added 1M LiHMDS in THF (1.1 mL, 1.658 mmol), and the reaction mixture was stirred for 10 min. Then, 5-(3H-[1,2,3]triazolo[4,5-b]pyridin-3-yl)-3-fluoropicolinoylchloride (200 mg, 0.663 mmol) in THF (5 mL) was added to reaction mixture. The reaction mixture was stirred at 0° C. for 20 min, and then allowed to warm to room temperature and stirred for 2 h. The reaction mixture was then diluted with water and extracted with EtOAc (2×50 mL). The combined organic layer was washed with brine, dried over Na₂SO₄, and concentrated under reduced pressure. The residue was purified by column chromatography, eluting with 80% EtOAc in hexane to afford tert-butyl (R)-3-(5-(3H-[1,2,3]triazolo[4,5-b]pyridin-3-yl)-N-(8-chloroisoquinolin-1-yl)-3-fluoropicolinamido)piperidine-1-carboxylate (50 mg, 15%) as an off-white solid. LC-MS: [M-Boc+H⁺]=502.7.

### Step 3:

The title compound hydrochloride salt was obtained as an off-white solid by deprotection Method A used in Example 97. LC-MS: [M+H⁺]=502.7. ¹H NMR (CD₃OD, 400 MHZ): δ 8.65 (bs, 2H), 8.41-8.39 (m, 3H), 7.72-7.60 (m, 3H), 7.47-7.41 (m, 2H), 5.05-4.98 (m, 1H), 3.92-3.90 (m, 1H), 3.70-3.65 (m, 1H), 3.30-3.27 (m, 1H), 2.90-2.88 (m, 1H), 2.00-1.85 (m, 3H), 1.42-1.39 (m, 1H).

### Synthesis of (R)-5-(3H-[1,2,3]triazolo[4,5-b]pyridin-3-yl)-3-fluoro-N-(8-methylisoquinolin-1-yl)-N-(piperidin-3-yl)picolinamide (Example 201)

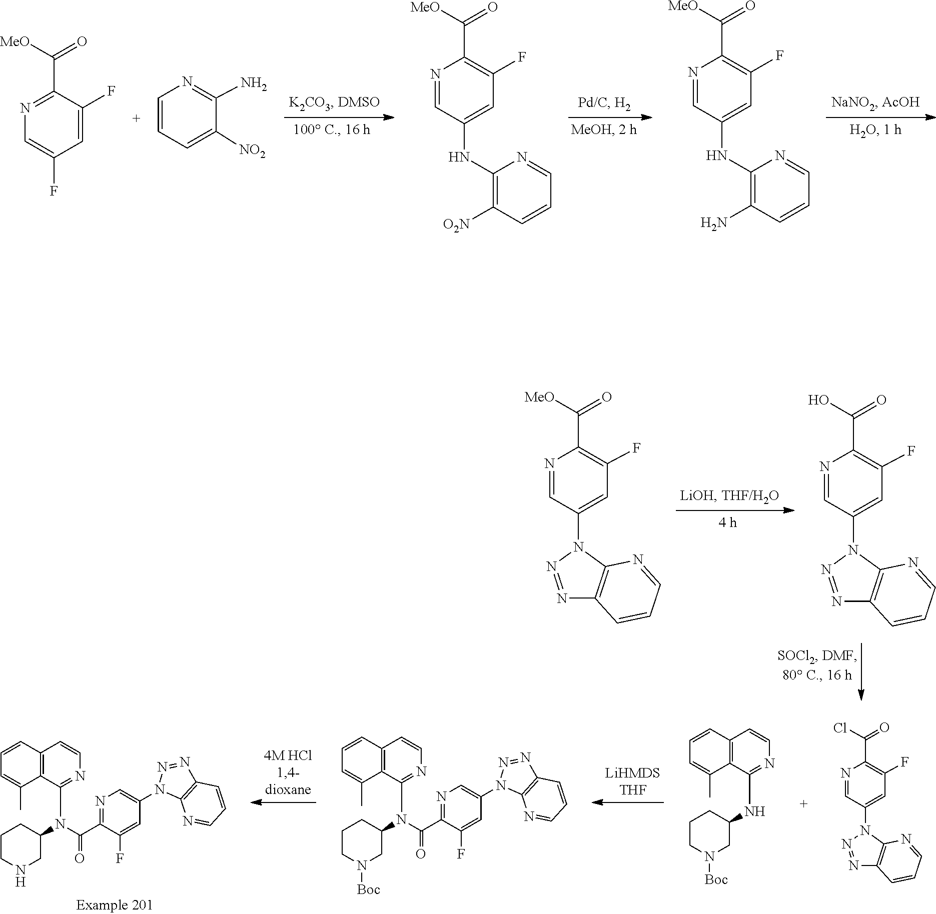

### Step 1:

To a stirred solution of methyl 3,5-difluoropicolinate (10 g, 57.763 mmol) in DMSO at room temperature (100 mL) were added 3-nitropyridin-2-amine (8.0 g, 57.763 mmol) and K₂CO₃ (15.9 g, 115.526 mmol). The reaction mixture was heated to 100° C. and stirred for 16 h. The reaction mixture was then concentrated and the residue was diluted with water (100 mL) and extracted with EtOAc (2×100 mL). The combined organic layer was dried over Na₂SO₄ and concentrated under vacuum. The residue was purified by Combi-flash using 40% EtOAc in hexane to obtain methyl 3-fluoro-5-((3-nitropyridin-2-yl)amino) picolinate (4.0 g, 24%) as a yellow solid. LC-MS: [M+H⁺]=292.8.

### Step 2:

To a stirred solution of methyl 3-fluoro-5-((3-nitropyridin-2-yl)amino) picolinate (100 mg, 0.342 mmol in MeOH (8 mL) was added Pd/C (10 wt %, 20 mg). The reaction was stirred at room temperature for 2 h under a hydrogen balloon atmosphere. The reaction mixture was then filtered through celite and the filtrate was concentrated to give crude methyl 5-((3-aminopyridin-2-yl)amino)-3-fluoropicolinate (80 mg) as an off-white solid. LC-MS: [M+H⁺]=262.9.

### Step 3:

To a stirred solution of methyl 5-((3-aminopyridin-2-yl)amino)-3-fluoropicolinate (0.8 g, 3.050 mmol) in a mixture of AcOH and H₂O (v:v=1:1, 20 mL) was added NaNO₂ (0.27 g, 3.965 mmol). The reaction mixture was stirred at room temperature for 1 h before it was then poured into ice water (20 mL). The resulting solid was collected by filtration and purified by Combi-flash using 60% EtOAc in hexane to obtain methyl 5-(3H-[1,2,3]triazolo[4,5-b]pyridin-3-yl)-3-fluoropicolinate (0.6 g, 75%) as a brown solid. LC-MS: [M+H⁺]=273.8.

### Step 4:

To a stirred solution of methyl 5-(3H-[1,2,3]triazolo[4,5-b]pyridin-3-yl)-3-fluoropicolinate (0.6 g, 2.196 mmol.) in 1:1 THF/H₂O (10 mL) was added LiOH (0.18 g, 4.392 mmol). The reaction mixture was stirred at room temperature for 4 h before it was concentrated. The residue was re-dissolved in water (0.5 mL) and acidified to pH 2 with 1N aq. HCl (2 mL). The resulting solid was collected by filtration and dried to yield 5-(3H-[1,2,3]triazolo[4,5-b]pyridin-3-yl)-3-fluoropicolinic acid (0.55 g, 96%) as an off-white solid. LC-MS: [M+H⁺]=260.1.

### Step 5:

To a solution of 5-(3H-[1,2,3]triazolo[4,5-b]pyridin-3-yl)-3-fluoropicolinic acid (550 mg, 2.121 mmol) in SOCl₂ (10 mL) at 0° C. was added a catalytic amount of DMF (0.1 mL). The reaction mixture was heated to 80° C. and stirred for 16 h. The reaction mixture was then concentrated under reduced pressure to afford crude 5-(3H-[1,2,3]triazolo[4,5-b]pyridin-3-yl)-3-fluoropicolinoyl chloride (600 mg) as a solid.

### Step 6:

To a solution of tert-butyl (R)-3-((8-methylisoquinolin-1-yl)amino)piperidine-1-carboxylate 4 (200 mg, 0.586 mmol) in THF (10 mL) at 0° C. was added 1M LiHMDS in THF (1.1 mL, 1.758 mmol). The reaction mixture was stirred for 10 min and followed by addition of 5-(3H-[1,2,3]triazolo[4,5-b]pyridin-3-yl)-3-fluoropicolinoyl chloride (190 mg, 0.703 mmol) in THF (2 mL). The reaction was stirred at 0° C. for 20 min, and then allowed to warm to room temperature and stir for another 2 h. The reaction mixture was quenched with water (50 mL) and extracted with ethyl acetate (2×50 mL). The combined organic layer was washed with brine (20 mL), dried over Na₂SO₄, and concentrated under reduced pressure. The residue was purified by Combi-flash using 40% EtOAc in hexane first and then was further purified by Prep-HPLC (Conditions: Column: Luna OMEGA C-18 (250×21.2×5 μm), Mobile phase A: 10 mM ammonium acetate in water, Mobile phase B: MeCN (100%), Gradient: T/% B: 0/65, 10/90, Flow: 15 mL/min, Dilution: MeCN+H₂O+THF. Pure fractions were dried by lyophilization to afford tert-butyl (R)-3-(5-(3H-[1,2,3]triazolo[4,5-b]pyridin-3-yl)-3-fluoro-N-(8-methylisoquinolin-1-yl)picolinamido)piperidine-1-carboxylate 5 (90 mg, 10%) as an off-white solid. LC-MS: [M+H⁺]=582.7.

### Step 7:

The title compound hydrochloride salt was obtained as an off-white solid by deprotection Method A used in Example 97. LC-MS: [M+H⁺]=483.3. ¹H NMR (CD₃OD, 400 MHZ): δ 8.83 (bs, 1H), 8.72 (d, J=4.4 Hz, 1H), 8.53 (dd, J=10.4 Hz, 1.6 Hz, 1H), 8.48 (d, J=8.4 Hz, 1H), 8.30 (d, J=5.6 Hz, 1H), 7.79-7.67 (m, 2H), 7.63-7.49 (m, 3H), 5.02-4.94 (m, 1H), 4.02 (dd, J=11.6 Hz, 2.4 Hz, 1H), 3.63 (t, J=11.2 Hz, 1H), 3.33-3.30 (m, 1H), 2.99-2.87 (m, 1H), 2.93 (s, 3H), 1.94-1.84 (m, 3H), 1.30-1.19 (m, 1H).

### Synthesis of (R)-2-fluoro-N-(8-methylisoquinolin-1-yl)-4-(6-methylpyrazin-2-yl)-N-(piperidin-3-yl)benzamide (Example 202)

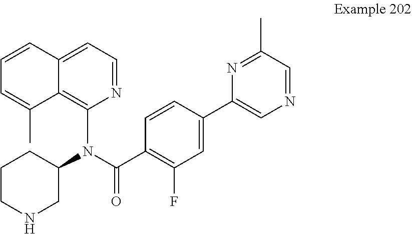 [SMILES: Cc1cncc(-c2ccc(C(=O)N(c3nccc4cccc(C)c34)[C@@H]3CCCNC3)c(F)c2)n1]

Example 202 was prepared according to the procedure described for Example 203 by substituting 2-bromo-3-methylpyrazine with 2-bromo-6-methylpyrazine, provided the title compound hydrochloride salt as an off-white solid. LC-MS: [M+H⁺]=456.3. ¹H NMR (CD₃OD, 400 MHZ): δ 8.61 (bs, 1H), 8.39-8.31 (m, 2H), 7.64-7.60 (m, 1H), 7.55-7.50 (m, 1H), 7.48-7.23 (m, 4H), 6.87-6.82 (m, 1H), 5.00-4.87 (m, 1H), 3.93-3.87 (m, 1H), 3.76-3.70 (m, 1H), 3.35-3.29 (m, 1H), 2.95-2.91 (m, 1H), 2.76 (s, 3H), 2.63-2.57 (m, 1H), 2.43 (s, 3H), 1.88-1.82 (m, 2H), 1.52-1.48 (m, 1H).

### Synthesis of (R)-2-fluoro-N-(8-methylisoquinolin-1-yl)-4-(3-methylpyrazin-2-yl)-N-(piperidin-3-yl)benzamide (Example 203)

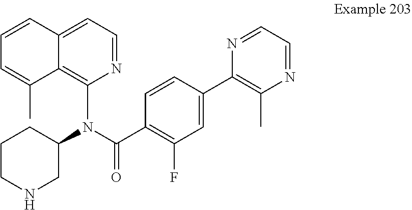 [SMILES: Cc1nccnc1-c1ccc(C(=O)N(c2nccc3cccc(C)c23)[C@@H]2CCCNC2)c(F)c1]

Example 203 was prepared according to the procedure described for Example 127 by substituting 2-bromo-5-methyl-1,3,4-thiadiazole with 2-bromo-3-methylpyrazine, provided the title compound hydrochloride salt as a white solid. LC-MS [M+H⁺]=456.3. ¹H NMR (CD₃OD, 400 MHZ): δ 8.58 (bs, 1H), 8.46 (bs, 1H), 8.35-8.31 (m, 1H), 7.69-7.54 (m, 2H), 7.45-7.37 (m, 2H), 7.01-6.88 (m, 3H), 5.03-4.97 (m, 1H), 3.95-3.92 (m, 1H), 3.79-3.73 (m, 1H), 3.34-3.26 (m, 1H), 3.00-2.88 (m, 1H), 2.76 (s, 3H), 2.65-2.64 (m, 1H), 2.26 (s, 3H), 1.88-1.85 (m, 2H), 1.59-1.53 (m, 1H).

### (R)-2-fluoro-N-(8-methylisoquinolin-1-yl)-N-(piperidin-3-yl)-4-(pyrazin-2-yl)benzamide (Example 204)

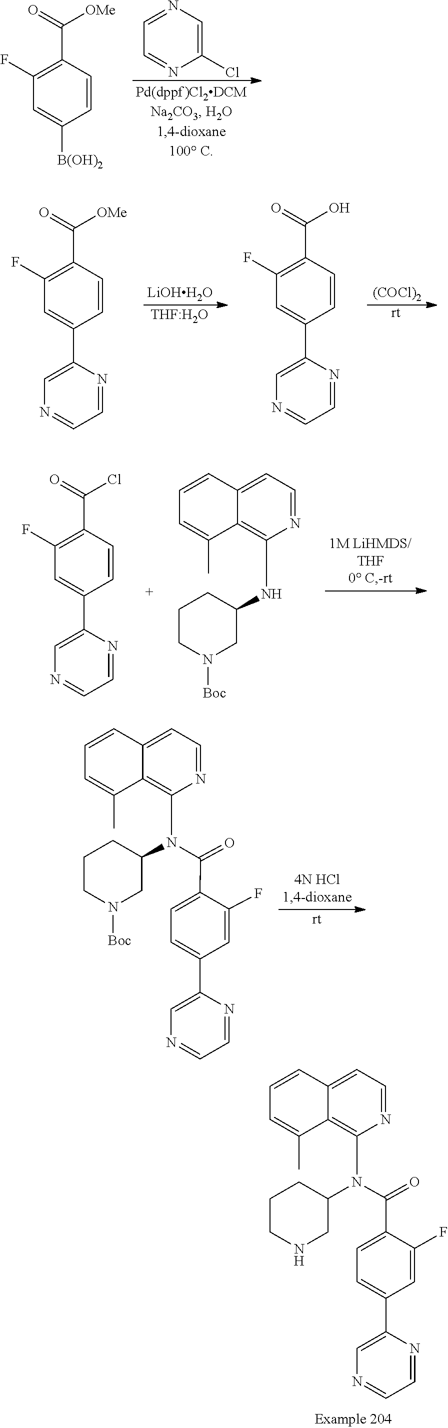

### Step 1:

To a mixture of 2-chloropyrazine (1.8 g, 15.720 mmol) and (3-fluoro-4-(methoxycarbonyl)phenyl)boronic acid (3.73 g, 18.864 mmol) in 1,4-dioxane (20 mL) and H₂O (20 mL) at room temperature was added NaHCO₃ (4.99 g, 47.161 mmol). The reaction mixture was purged with nitrogen for 10 min and followed by addition of Pd(dppf)·CH₂Cl₂ (1.28 g, 1.572 mmol). The reaction mixture was then stirred at 100° C. for 4 h before it was cooled, diluted with water (100 mL) and extracted with ethyl acetate (2×200 mL). The combined organic layer was washed with brine and concentrated under reduced pressure. The residue was purified by Combi-flash using 40% EtOAc in hexane to afford methyl 2-fluoro-4-(pyrazin-2-yl)benzoate (2.1 g, 58%) as an off-white solid.

### Step 2:

To a solution of methyl 2-fluoro-4-(pyrazin-2-yl)benzoate (2.1 g, 9.043 mmol) in THF (10 mL), MeOH (10 mL) and H₂O (10 mL) at room temperature was added LiOH (1.89 g, 45.217 mmol). The reaction mixture stirred for 16 h before it was acidified with 2N aq. HCl and extracted with EtOAc (2×100 mL). The combined organic layer was dried over Na₂SO₄ and concentrated under reduced pressure to afford 2-fluoro-4-(pyrazin-2-yl)benzoic acid (1.7 g, 90%) as an off-white solid. LC-MS: [M+H⁺]=219.35.

### Step 3:

To a stirred solution of 2-fluoro-4-(pyrazin-2-yl)benzoic acid (200 mg, 0.916 mmol) in DCM (10 mL) at 0° C. was added oxalyl chloride (0.11 mL, 1.374 mmol) and DMF (0.1 mL). The reaction mixture was stirred at room temperature for 2 h. The reaction mixture was concentrated under reduced pressure to afford crude 2-fluoro-4-(pyrazin-2-yl)benzoyl chloride (200 mg) as a solid.

### Step 4:

To a stirred solution of tert-butyl (R)-3-((8-methylisoquinolin-1-yl)amino)piperidine-1-carboxylate (200 mg, 0.586 mmol) in THF (10 mL) at 0° C. was added 1.5M LiHMDS in THF (0.5 mL, 0.879 mmol). The reaction was stirred for 10 min and followed by addition of 2-fluoro-4-(pyrazin-2-yl)benzoyl chloride (83.2 mg, 0.351 mmol) in THF (2 mL). The reaction was stirred for 20 min before it was diluted with water (30 mL) and extracted with EtOAc (2×50 mL). The combined organic layer was dried over Na₂SO₄ and concentrated. The mixture was purified by Prep-HPLC (Conditions: Column: Luna OMEGA C-18 (250×21.2×5 μm), Mobile phase A: 0.1% formic acid in water, Mobile phase B: 100% MeCN, Gradient: Time (min)/% B: 0/65, 10/95, Flow: 15 mL/min, Dilution: MeCN+H₂O+THF). Pure fractions were combined and dried by lyophilization to afford tert-butyl (R)-3-(2-fluoro-N-(8-methylisoquinolin-1-yl)-4-(pyrazin-2-yl)benzamido)piperidine-1-carboxylate (120 mg) as a white-solid. LC-MS: [M+H⁺]=542.9.

### Step 5:

The title compound hydrochloride salt was obtained as an off-white solid by deprotection Method A used in Example 97. LC-MS: [M+H⁺]=442.6. ¹H NMR (CD₃OD, 400 MHz) δ 8.85 (bs, 1H), 8.55 (bs, 1H), 8.43 (d, J=2.4 Hz, 1H), 8.35-8.31 (m, 1H), 7.66-7.61 (m, 1H), 7.56-7.48 (m, 2H), 7.45-7.36 (m, 3H), 6.92-6.83 (m, 1H), 5.00-4.95 (m, 1H), 3.95-3.91 (m, 1H), 3.76-3.70 (m, 1H), 3.33-3.25 (m, 1H), 2.96-2.91 (m, 1H), 2.79 (s, 3H), 2.65-2.62 (m, 1H), 1.88-1.82 (m, 2H), 1.49-1.45 (m, 1H), 1.25-1.18 (m, 1H).

### Synthesis of (R)-2-fluoro-N-(8-methylisoquinolin-1-yl)-N-(piperidin-3-yl)-4-(5-(trifluoromethyl)-3H-[1,2,3]triazolo[4,5-b]pyridin-3-yl)benzamide (Example 205)

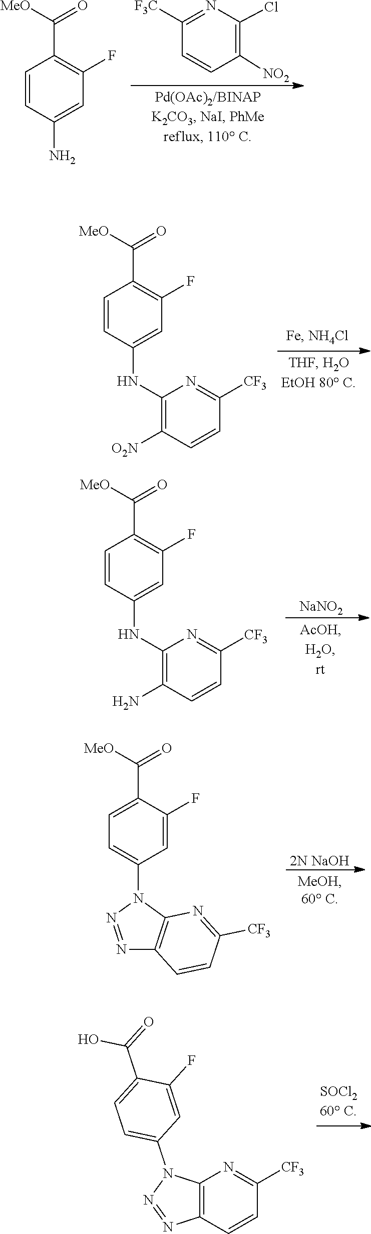

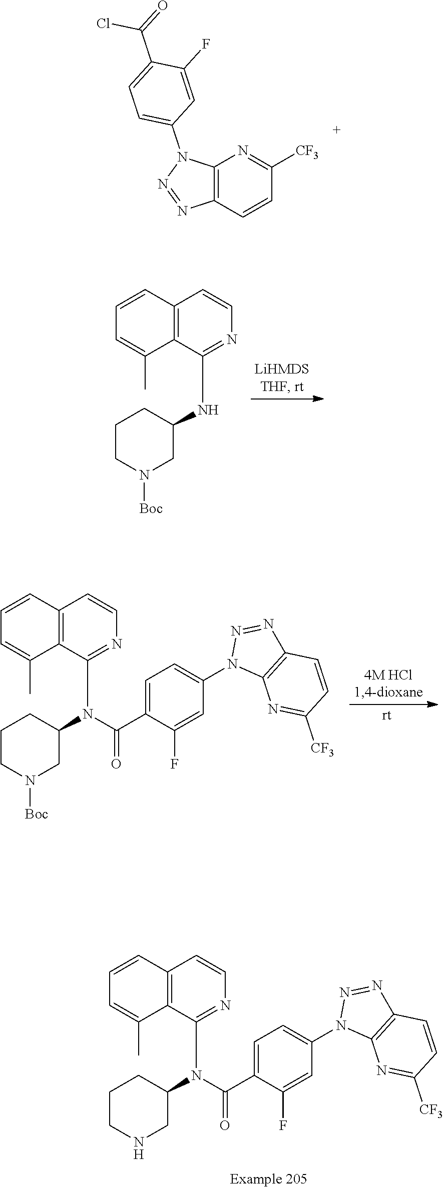 [SMILES: O=C(Cl)c1ccc(-n2nnc3ccc(C(F)(F)F)nc32)cc1F ; Cc1cccc2ccnc(N[C@@H]3CCCN(C(=O)OC(C)(C)C)C3)c12 ; Cc1cccc2ccnc(N(C(=O)c3ccc(-n4nnc5ccc(C(F)(F)F)nc54)cc3F)[C@@H]3CCCN(C(=O)OC(C)(C)C)C3)c12 ; Cc1cccc2ccnc(N(C(=O)c3ccc(-n4nnc5ccc(C(F)(F)F)nc54)cc3F)[C@@H]3CCCNC3)c12]

Example 205 was prepared according to the procedure described for Example 192 by substituting 2,6-di-chloro-3-nitropyridine with 2-chloro-3-nitro-6-(trifluoromethyl)pyridine, provided the title compound hydrochloride salt as a white solid.

LC-MS: [M+H⁺]=551.35. ¹H NMR (CD₃OD, 400 MHZ) δ 8.69 (d, J=7.2 Hz, 1H), 8.38-8.34 (m, 1H), 7.87-7.83 (m, 2H), 7.72-7.70 (m, 1H), 7.65-7.61 (m, 1H), 7.54-7.53 (m, 1H), 7.42-7.38 (m, 2H), 7.07-7.02 (m, 1H), 5.03-4.96 (m, 1H), 4.12-4.10 (m, 1H), 3.93-3.92 (m, 1H), 3.77-3.72 (m, 1H), 3.38-3.24 (m, 1H), 2.96-2.88 (m, 1H), 2.78 (s, 3H), 1.92-1.83 (m, 2H), 1.25-1.19 (m, 1H).

### Synthesis of (S,E)-4-(4-(4-(dimethylamino)but-2-enamido)phenyl)-N-(1-methyl-1H-pyrrolo[2,3-c]pyridin-7-yl)-N-(piperidin-3-yl)piperidine-1-carboxamide (Example 206)

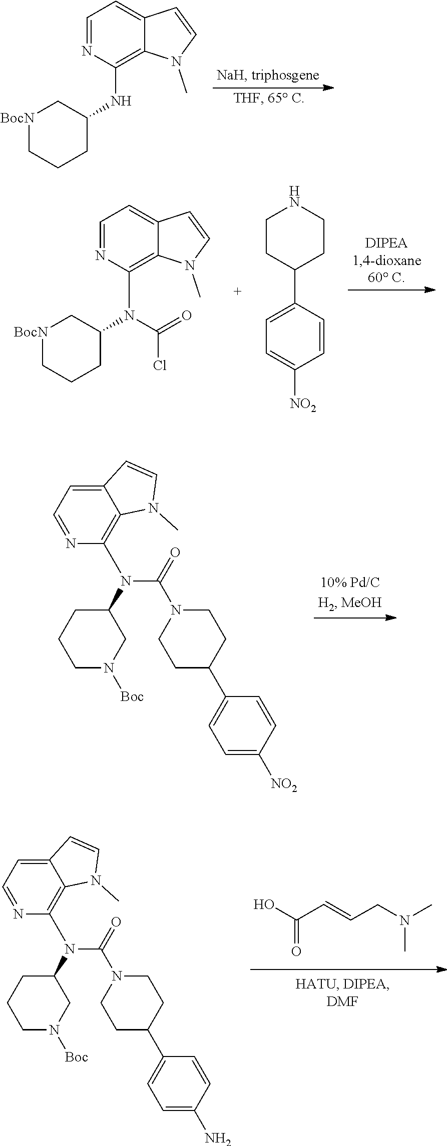

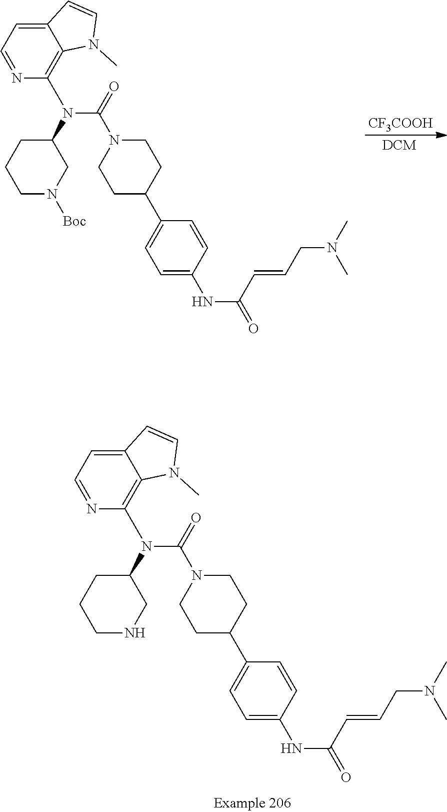 [SMILES: CN(C)C/C=C/C(=O)Nc1ccc(C2CCN(C(=O)N(c3nccc4ccn(C)c34)[C@@H]3CCCN(C(=O)OC(C)(C)C)C3)CC2)cc1 ; "CF3COOH" ; CN(C)C/C=C/C(=O)Nc1ccc(C2CCN(C(=O)N(c3nccc4ccn(C)c34)[C@@H]3CCCNC3)CC2)cc1]

### Step 1:

To a stirred solution of tert-butyl (R)-3-((1-methyl-1H-pyrrolo[2,3-c]pyridin-7-yl) amino) piperidine-1-carboxylate (0.5 g, 1.51 mmol, 1 eq.) in THF (10 mL) at room temperature was added NaH (60% in mineral oil, 84 mg, 2.11 mmol). The reaction was stirred at room temperature for 30 min and followed by addition of triphosgene (314 mg, 1.05 mmol) in toluene (1 mL). The reaction mixture was heated to 60° C. and stirred for 1 h before it was cooled. The reaction mixture was diluted with water (50 mL) and extracted with EtOAc (2×100 mL). The combined organic layer was dried over Na₂SO₄ and concentrated under reduced pressure. The residue was purified by Combi-flash using 20% EtOAc in hexane to afford tert-butyl (R)-3-((chlorocarbonyl)(1-methyl-1H-pyrrolo[2,3-c]pyridin-7-yl) amino)piperidine-1-carboxylate 2 (282 mg, 47%) as a yellowish liquid. LC-MS: [M+H⁺]=393.10.

### Step 2:

To a stirred solution of 4-(4-nitrophenyl)piperidine (450 mg, 2.18 mmol) and DIPEA (1.05 ml, 5.94 mmol) in 1,4-dioxane (12 mL) at room temperature was added tert-butyl (R)-3-((chlorocarbonyl)(1-methyl-1H-pyrrolo[2,3-c]pyridin-7-yl)amino)piperidine-1-carboxylate (780 mg, 1.98 mmol). The reaction mixture was heated to 100° C. and stirred for 4 h before it was allowed to cool. The reaction mixture was diluted with water (60 mL) and extracted with EtOAc (2×150 mL). The combined organic layer was washed with brine (50 mL), dried over Na₂SO₄, and concentrated under reduced pressure. The residue was purified by Combi-flash using 40% EtOAc in hexane to afford tert-butyl (R)-3-(N-(1-methyl-1H-pyrrolo[2,3-c]pyridin-7-yl)-4-(4-nitrophenyl)piperidine-1-carboxamido)piperidine-1-carboxylate (680 mg, 61%) as an off-white solid. LC-MS: [M+H⁺]=563.35.

### Step 3:

To a stirred solution of tert-butyl (R)-3-(N-(1-methyl-1H-pyrrolo[2,3-c]pyridin-7-yl)-4-(4-nitrophenyl)piperidine-1-carboxamido)piperidine-1-carboxylate (680 mg, 1.20 mmol) in ethanol (20 mL) at room temperature was added Pd/C (10 wt %, 400 mg). The reaction mixture was stirred under a hydrogen atmosphere (50 psi) for 3 h. The reaction mixture was filtered through a pad of celite, and the celite was washed with ethanol. The combined filtrate was concentrated under reduced pressure to afford crude tert-butyl (R)-3-(4-(4-aminophenyl)-N-(1-methyl-1H-pyrrolo[2,3-c]pyridin-7-yl)piperidine-1-carboxamido)piperidine-1-carboxylate (600 mg, 93%) as a yellowish liquid. LC-MS: [M+H⁺]=533.35.

### Step 4:

To a solution of tert-butyl(S)-3-(4-(4-aminophenyl)-N-(1-methyl-1H-pyrrolo[2,3-c]pyridin-7-yl)piperidine-1-carboxamido)piperidine-1-carboxylate (200 mg, 0.375 mmol) in DMF (2 mL) at room temperature was added DIPEA (0.2 mL, 1.126 mmol), HATU (280 mg, 0.736 mmol) and (E)-4-(dimethylamino)but-2-enoic acid (96 mg, 0.743 mmol). The reaction mixture was stirred at room temperature for 2 h, after which it was diluted with water (50 mL) and extracted with ethyl acetate (2×80 mL). The combined organic layer was washed with brine, dried over Na₂SO₄, and concentrated under reduced pressure. The residue was purified by Prep-HPLC (Conditions: Column: Luna OMEGA C-18 (250×21.2×5 μm), Mobile phase A: 0.1% formic acid in water, Mobile phase B: 100% MeCN, Gradient: T/% B: 0/20, 3/20, 10/50, Flow: 15 mL/min, Dilution: MeCN+H₂O+THF). Pure fractions were dried by lyophilization to afford tert-butyl (S,E)-3-(4-(4-(4-(dimethylamino)but-2-enamido)phenyl)-N-(1-methyl-1H-pyrrolo[2,3-c]pyridin-7-yl)piperidine-1-carboxamido)piperidine-1-carboxylate (60 mg, 25%) as an off-white solid. LC-MS: [M+H⁺]=644.4.

### Step 5:

To a solution of tert-butyl (S,E)-3-(4-(4-(4-(dimethylamino)but-2-enamido) phenyl)-N-(1-methyl-1H-pyrrolo[2,3-c]pyridin-7-yl)piperidine-1-carboxamido)piperidine-1-carboxylate (30 mg, 0.046 mmol) in DCM (3 mL) at 0° C. was added trifluoroacetic acid (0.2 mL). The reaction mixture was warmed to room temperature and stirred for 1 h. The reaction mixture was concentrated under reduced pressure, and the residue was purified by Prep-HPLC (Conditions: Column: Luna OMEGA C-18 (250×21.2×5 μm), Mobile phase A: 0.1% TFA in water, Mobile phase B: 100% MeCN, Gradient: T/% B: 0/10, 2/10, 10/50, Flow: 15 mL/min, Dilution: MeCN+H₂O+THF). Pure fractions were dried by lyophilization for 2 days to afford (S,E)-4-(4-(4-(dimethylamino)but-2-enamido)phenyl)-N-(1-methyl-1H-pyrrolo[2,3-c]pyridin-7-yl)-N-(piperidin-3-yl)piperidine-1-carboxamide trifluoroacetic acid salt (14 mg, 28%) as an off-white solid. LC-MS: [M+H⁺]=544.3. ¹H NMR (DMSO-d₆ at 90° C., 400 MHz): δ 10.21 (s, 1H), 9.71 (m, 1H), 8.69 (m, 1H), 8.02 (d, J=5.2 Hz, 1H), 7.67 (d, J=2.8 Hz, 1H), 7.50 (t, J=5.6 Hz, 3H), 6.86 (m, 1H), 6.72-6.66 (m, 1H), 6.61 (d, J=3.2 Hz, 1H), 6.41 (d, J=15.2 Hz, 1H), 4.52 (m 1H), 3.91 (d, J=6.0 Hz, 2H), 3.86 (s, 3H), 3.57-3.55 (m, 2H), 3.45-3.43 (m, 3H), 2.80 (s, 6H), 1.90-1.84 (m, 6H), 1.46 (m, 2H), 1.24 (m, 2H), 0.91-0.86 (m, 2H).

### Synthesis of (R,E)-4-(3-(4-(dimethylamino)but-2-enamido)phenyl)-N-(8-methylisoquinolin-1-yl)-N-(piperidin-3-yl)piperidine-1-carboxamide (Example 207)

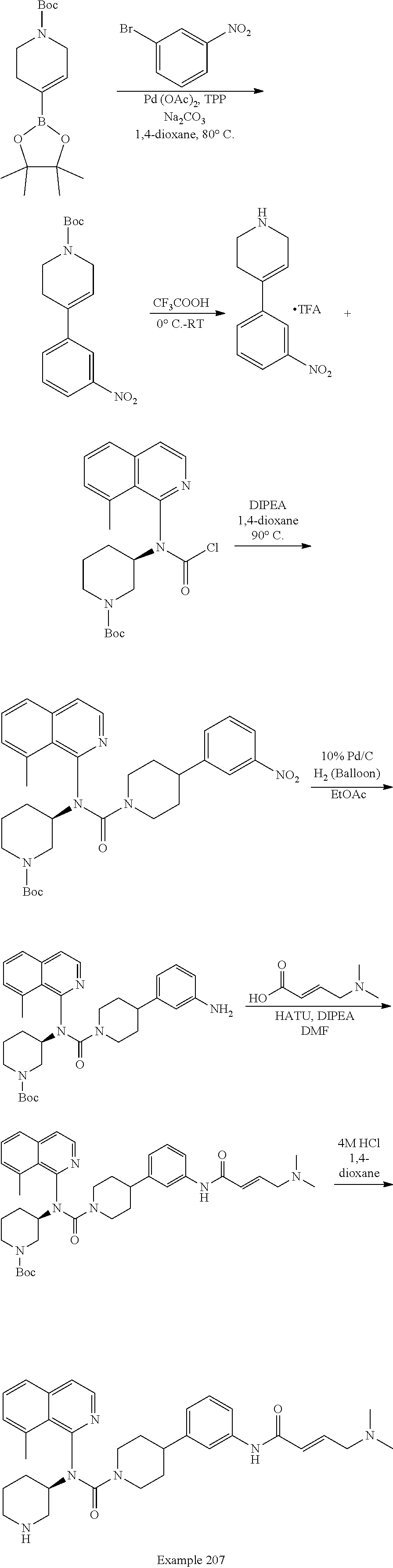

### Step 1:

To a solution of 1-bromo-3-nitrobenzene (5.03 g, 24.90 mmol), tert-butyl 4-(4,4,5,5-tetramethyl-1,3,2-dioxaborolan-2-yl)-3,6-dihydropyridine-1(2H)-carboxylate (7.7 g, 24.90 mmol) in 1,4 dioxane (70 mL) at room temperature were added 1.5 M aq. Na₂CO₃ (6.60 g, 62.25 mmol) and PPh₃ (3.91 g, 14.94 mmol). The reaction mixture was degassed with nitrogen for 10 min and followed by addition of Pd(OAc)₂ (560 mg, 2.49 mmol). The resulting mixture was again degassed with nitrogen for 5 min and then stirred at 80° C. for 16 h. The reaction mixture was diluted with water (100 mL) and extracted with DCM (2×150 mL). The combined organic layer was dried over Na₂SO₄ and concentrated under reduced pressure. The residue was purified by Combi-flash, eluting with 10-20% EtOAc in hexane to afford tert-butyl 4-(3-nitrophenyl)-3,6-dihydropyridine-1(2H)-carboxylate (5.5 g, 73%) as yellow gummy solid.

### Step 2:

A solution of tert-butyl 4-(3-nitrophenyl)-3,6-dihydropyridine-1(2H)-carboxylate (5.5 g, 18.07 mmol) in TFA (40 mL) was stirred at room temperature for 16 h. The reaction mixture was concentrated under reduced pressure, washed with diethyl ether, and dried to afford 4-(3-nitrophenyl)-1,2,3,6-tetrahydropyridine trifluoroacetic acid salt (5.4 g, 99%) as an off-white solid. LC-MS: [M+H⁺]=205.

### Step 3:

To a solution of tert-butyl (R)-3-((chlorocarbonyl)(8-methylisoquinolin-1-yl)amino)piperidine-1-carboxylate (150 mg, 0.37 mmol) in 1,4-dioxane (2 mL) were added 4-(3-nitrophenyl)-1,2,3,6-tetrahydropyridine (168 mg, 0.55 mmol) and then DIPEA (0.5 mL, 1.11 mmol). The reaction mixture was stirred at 90° C. for 16 h before it was diluted with water and extracted with EtOAc (2×50 mL). The combined organic layer was dried over Na₂SO₄ and concentrated. The residue was purified by Combi-flash using 40% EtOAc in hexane to afford tert-butyl (R)-3-(N-(8-methylisoquinolin-1-yl)-4-(3-nitrophenyl)piperidine-1-carboxamido)-piperidine-1-carboxylate (200 mg, 94%) as an off-white solid. LC-MS: [M+H⁺]=572.2.

### Step 4:

To a solution of tert-butyl (R)-3-(N-(8-methylisoquinolin-1-yl)-4-(3-nitrophenyl)-1,2,3,6-tetrahydropyridine-1-carboxamido)piperidine-1-carboxylate (200 mg, 0.35 mmol) in EtOH (10 mL) was added Pd/C (10 wt %, 100 mg). The reaction was stirred at room temperature under a under H₂ balloon (40 Psi) for 16 h. The reaction mixture was then filtered through a pad of celite and rinsed with ethyl acetate. The combined filtrate was concentrated under reduced pressure. The residue was purified by Combi-flash using 70% EtOAc in hexane to afford tert-butyl (R)-3-(4-(3-aminophenyl)-N-(8-methylisoquinolin-1-yl)piperidine-1-carboxamido)piperidine-1-carboxylate (130 mg, 68%) as an off-white solid. LC-MS: [M+H⁺]=544.3.

### Step 5:

To a stirred solution of tert-butyl (R)-3-(4-(3-aminophenyl)-N-(8-methylisoquinolin-1-yl)piperidine-1-carboxamido)piperidine-1-carboxylate (100 mg, 0.18 mmol) and (E)-4-(dimethylamino)but-2-enoic acid hydrochloride (140 mg, 0.27 mmol) in DMF (2 mL) at 0° C. were added DIPEA (0.2 mL, 0.54 mmol) followed by HATU (140 mg, 0.36 mmol). The reaction mixture was allowed to warm to room temperature and then stirred for 16 h. The reaction mixture was diluted with water (15 mL) and extracted with EtOAc (2×50 mL). The combined organic layer was dried over Na₂SO₄ and concentrated under reduced pressure. The residue was purified by Combi-flash using 5% MeOH in DCM to afford tert-butyl (R,E)-3-(4-(3-(4-(dimethylamino)but-2-enamido)phenyl)-N-(8-methylisoquinolin-1-yl)piperidine-1-carboxamido)piperidine-1-carboxylate (40 mg) as an off-white solid. LC-MS: [M+H⁺]=655.7.

### Step 6:

To a solution of tert-butyl (R,E)-3-(4-(3-(4-(dimethylamino)but-2-enamido)phenyl)-N-(8-methylisoquinolin-1-yl)piperidine-1-carboxamido)piperidine-1-carboxylate (40 mg, 0.06 mmol) in DCM (0.5 mL) at 0° C. was added 4M HCl in 1,4-dioxane (0.3 mL). The reaction mixture was stirred for 2 h, after which it was concentrated under reduced pressure. The residue was purified by Prep-HPLC (Conditions: Column: XBRIDGE BEH AMIDE OBD (250×19×5 μm), Mobile phase A: 0.1% formic acid in water, Mobile phase B: 100% MeCN, Gradient: T/% B: 0/25, 10/50. Flow: 15 mL/min, Dilution: MeCN+H₂O+THF). The pure fractions were lyophilized to afford (R,E)-4-(3-(4-(dimethylamino)but-2-enamido)phenyl)-N-(8-methylisoquinolin-1-yl)-N-(piperidin-3-yl)piperidine-1-carboxamide formic acid salt (13 mg, 44%) as an off-white solid. LC-MS: [M+H⁺]=555.1. ¹H NMR (DMSO-d₆ at 90° C., 400 MHz) δ 11.15 (bs, 1H), 10.25 (bs, 1H), 8.36 (d, J=5.2 Hz, 1H), 7.84 (d, J=7.6 Hz, 1H), 7.74 (d, J=5.2 Hz, 1H), 7.66 (t, J=7.2 Hz, 1H), 7.53 (bs, 1H), 7.43-7.30 (m, 1H), 7.15-7.11 (m, 1H), 6.85-6.77 (m, 1H), 6.65-6.59 (m, 1H), 6.54-6.50 (m, 1H), 4.75 (m, 1H), 4.18-4.08 (m, 1H), 3.88 (s, 3H), 3.75-3.72 (m, 2H), 3.09-3.05 (m, 3H), 2.88-2.82 (m, 3H), 2.66 (s, 6H), 2.43-2.36 (m, 3H), 1.81-1.76 (m, 3H), 1.44-1.33 (m, 1H), 1.24-1.13 (m, 1H), 0.94-0.78 (m, 1H), 0.30-0.22 (m, 1H).

### Synthesis of (R)—N-(8-methylisoquinolin-1-yl)-N-(piperidin-3-yl)-4-(pyrimidin-2-ylamino)piperidine-1-carboxamide (Example 208)

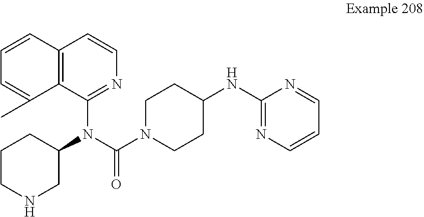 [SMILES: Cc1cccc2ccnc(N(C(=O)N3CCC(Nc4ncccn4)CC3)[C@@H]3CCCNC3)c12]

Example 208 was prepared according to the procedure described for Example 199 by substituting 2-methyl-4-(piperidin-4-yl)pyridine with N-4-piperidinyl-2-pyrimidinamine, provided the title compound hydrochloride salt as an off-white solid. LC-MS: [M+H⁺]=446.2.

### Synthesis of (R)-2-fluoro-N-(1-methyl-1H-pyrrolo[2,3-c]pyridin-7-yl)-4-(5-methyl-3H-[1,2,3]triazolo[4,5-b]pyridin-3-yl)-N-(piperidin-3-yl)benzamide (Example 209)

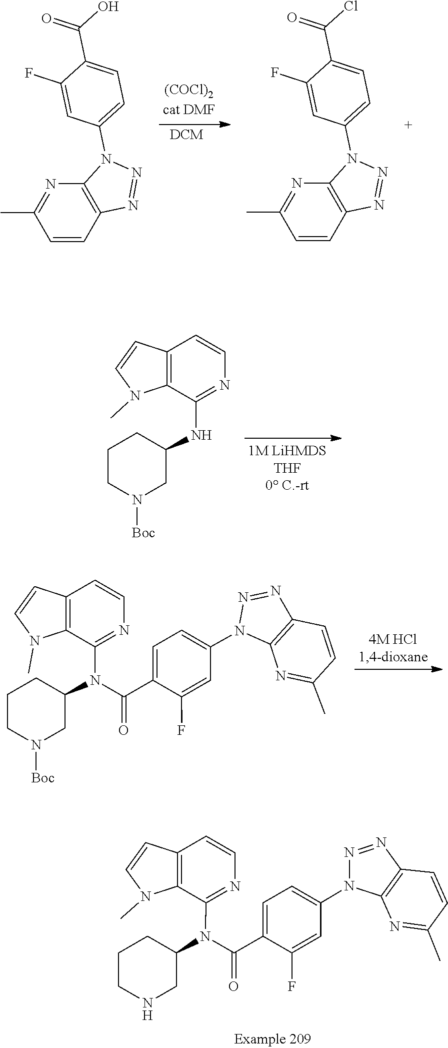 [SMILES: Cc1ccc2nnn(-c3ccc(C(=O)O)c(F)c3)c2n1 ; Cc1ccc2nnn(-c3ccc(C(=O)Cl)c(F)c3)c2n1 ; Cn1ccc2ccnc(N[C@@H]3CCCN(C(=O)OC(C)(C)C)C3)c21 ; Cc1ccc2nnn(-c3ccc(C(=O)N(c4nccc5ccn(C)c45)[C@@H]4CCCN(C(=O)OC(C)(C)C)C4)c(F)c3)c2n1 ; Cc1ccc2nnn(-c3ccc(C(=O)N(c4nccc5ccn(C)c45)[C@@H]4CCCNC4)c(F)c3)c2n1]

### Step 1:

To a stirred solution of 2-fluoro-4-(5-methyl-3H-[1,2,3]triazolo[4,5-b]pyridin-3-yl)benzoic acid (150 mg, 0.550 mmol, 1.0 eq.) in DCM (10 mL) at 0° C. were added oxalyl chloride (0.07 mL, 0826 mmol) and followed by a catalytic amount of DMF (0.1 mL). The reaction mixture was warmed to room temperature and stirred for 2 h. After completion of the reaction, the reaction mixture was concentrated under reduced pressure to afford crude 2-fluoro-4-(5-methyl-3H-[1,2,3]triazolo[4,5-b]pyridin-3-yl)benzoyl chloride (150 mg) as a gummy solid which was used directly in the next step.

### Step 2:

To a stirred solution of tert-butyl (R)-3-((8-methylisoquinolin-1-yl)amino)piperidine-1-carboxylate (200 mg, 0.585 mmol) in THF (6 mL) at 0° C. was added 1M LiHMDS in THF (1.17 mL, 1.17 mmol). The reaction mixture was stirred for 20 min and followed by addition of 2-fluoro-4-(5-methyl-3H-[1,2,3]triazolo[4,5-b]pyridin-3-yl)benzoyl chloride (170 mg, 0.585 mmol). The reaction was stirred at 0° C. for 10 min and then warmed to room temperature and stirred for 2 h. The reaction was quenched with water (30 mL) and extracted with EtOAc (2×50 mL). The combined organic layer was washed with brine (10 mL), dried over Na₂SO₄, and concentrated under reduced pressure. The residue was purified by Combi-flash using 50% EtOAc in hexane first and then further purified by Prep-HPLC (Conditions: Column: ZORBAX BONUS-RP (150×21.2×5 μm), Mobile phase A: 0.1% AA in water, Mobile phase B: 100% MeCN, Gradient: T/% B: 0/50, 10/80, Flow: 15 mL/min, Dilution: MeCN+H₂O+THF). Pure fractions were dried by lyophilization to afford tert-butyl (R)-3-(2-fluoro-N-(1-methyl-1H-pyrrolo[2,3-c]pyridin-7-yl)-4-(5-methyl-3H-[1,2,3]triazolo[4,5-b]pyridin-3-yl)benzamido)piperidine-1-carboxylate (65 mg, 20%) as an off-white solid. LC-MS: [M+H⁺]=585.3.

### Step 3:

The title compound hydrochloride salt was obtained as an off-white solid by deprotection Method A used in Example 97. LC-MS: [M+H⁺]=485.3. ¹H NMR (DMSO-d₆ at 90° C., 400 MHZ) δ 9.39-9.31 (m, 1H), 8.46-8.44 (m, 1H), 8.04-8.03 (m, 2H), 7.89-7.82 (m, 1H), 7.55-7.35 (m, 4H), 6.48-6.44 (m, 1H), 5.08-4.98 (m, 1H), 4.05 (m, 1H), 3.82-3.78 (m, 1H), 3.56-3.51 (m, 2H), 3.25-3.18 (m, 1H), 2.79-2.65 (m, 2H), 2.68 (s, 3H), 1.95-1.80 (m, 3H), 1.25-1.22 (m, 1H).

### Synthesis of (R)-2-fluoro-N-(8-methylisoquinolin-1-yl)-N-(piperidin-3-yl)-4-(pyrimidin-2-ylamino)benzamide (Example 210)

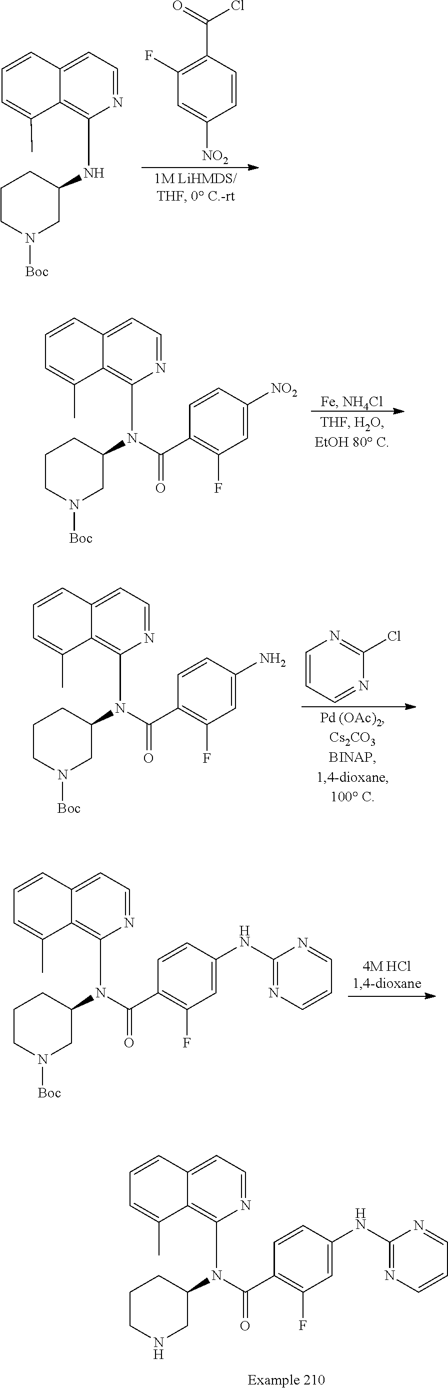

### Step 1:

To a solution of tert-butyl (R)-3-((8-methylisoquinolin-1-yl)amino)piperidine-1-carboxylate (300 mg, 0.87 mmol) in THF (5 mL) at 0° C. was added 1.4 M LiHMDS in THF (2.0 mL, 2.61 mmol). The reaction was stirred for 15 min and followed by addition of 2-fluoro-4-nitrobenzoyl chloride (270 mg, 1.30 mmol) in THF (5 mL). The reaction mixture was allowed to warm to room temperature and was stirred for 1 h. The reaction mixture was diluted with water and extracted with EtOAc (2×50 mL). The combined organic layer was dried over Na₂SO₄ and concentrated under reduced pressure. The residue was purified by Combi-flash using 40-50% EtOAc in hexane to afford tert-butyl (R)-3-(2-fluoro-N-(8-methylisoquinolin-1-yl)-4-nitrobenzamido)piperidine-1-carboxylate (150 mg, 33%) as a solid. LC-MS: [M+H⁺]=509.15.

### Step 2:

To a stirred solution of tert-butyl (R)-3-(2-fluoro-N-(8-methylisoquinolin-1-yl)-4-nitrobenzamido)piperidine 1-carboxylate (150 mg, 0.29 mmol) in THF (5 mL) and MeOH (5 mL) at 0° C. were added Fe powder (170 mg, 2.94 mmol) and followed by NH₄Cl (160 mg, 2.94 mmol) in H₂O (5 mL). The reaction mixture was heated to 80° C. and stirred for 2 h. After completion of the reaction, the reaction mixture was filtered through a pad of celite and was rinsed with MeOH (20 mL). The combined filtrate was concentrated under reduced pressure. The residue was diluted with water (20 mL) and extracted with EtOAc (2×30 mL). The combined organic layer was dried over Na₂SO₄ and concentrated under reduced pressure. The residue was purified by Combi-flash using 40-50% EtOAc in hexane to afford tert-butyl (R)-3-(4-amino-2-fluoro-N-(8-methylisoquinolin-1-yl)benzamido)piperidine-1-carboxylate (60 mg, 43%) as a pale yellow solid. LC-MS: [M+H⁺]=479.70.

### Step 3:

To a stirred solution of tert-butyl (R)-3-(4-amino-2-fluoro-N-(8-methylisoquinolin-1-yl)benzamido)piperidine-1-carboxylate (300 mg, 0.62 mmol) and 2-chloropyrimidine (150 mg, 1.25 mmol) in 1,4-dioxane (10 mL) at room temperature were added Cs₂CO₃ (700 mg, 1.86 mmol) followed by Rac BINAP (80 mg, 0.12 mmol). The reaction mixture was degassed with nitrogen for 10 min and followed by addition of Pd(OAc)₂ (14 mg, 0.06 mmol). The reaction mixture was degassed for 5 min and then heated to 100° C. and stirred for 16 h. After completion of the reaction, the reaction mixture was filtered through celite, and the celite was rinsed with ethyl acetate. The combined filtrate was concentrated under reduced pressure. The residue was purified by Combi-flash using 40-50% EtOAc in hexane. The product was further purified by Prep-HPLC (Conditions: Column: Luna OMEGA C-18 (250×21.2×5 μm), Mobile phase A: 10 mM ammonium acetate in water, Mobile phase B: 100% MeCN, Gradient: T/% B: 0/60, 10/85, Flow: 15 mL/min, Dilution: MeCN+H₂O+THF). Pure fractions were dried by lyophilization to afford tert-butyl (R)-3-(2-fluoro-N-(8-methylisoquinolin-1-yl)-4-(pyrimidin-2-ylamino)benzamido)piperidine-1-carboxylate (45 mg, 30%) as a pale yellow solid. LC-MS: [M+H⁺]=557.20.

### Step 4:

The title compound hydrochloride salt was obtained as an off-white solid by deprotection Method A used in Example 97. LC-MS: [M+H⁺]=457.65. ¹H NMR (DMSO-do at 90° C., 400 MHZ) δ 9.59 (bs, 1H), 8.45-8.39 (m, 3H), 7.77-7.69 (m, 2H), 7.54-7.45 (m, 3H), 6.97 (d, J=8.4 Hz, 1H), 6.86 (t, J=4.8 Hz, 1H), 6.64 (t, J=8.4 Hz, 1H), 4.94 (bs, 1H), 3.77-3.67 (m, 1H), 3.29-3.25 (m, 1H), 2.87-2.79 (m, 1H), 2.75 (s, 3H), 1.86-1.73 (m, 3H), 1.36-1.21 (m, 2H).

### Synthesis of (R)-2-fluoro-4-(1-methyl-1H-pyrazol-4-yl)-N-(1-methyl-1H-pyrrolo[2,3-c]pyridin-7-yl)-N-(piperidin-3-yl)benzamide (Example 211)

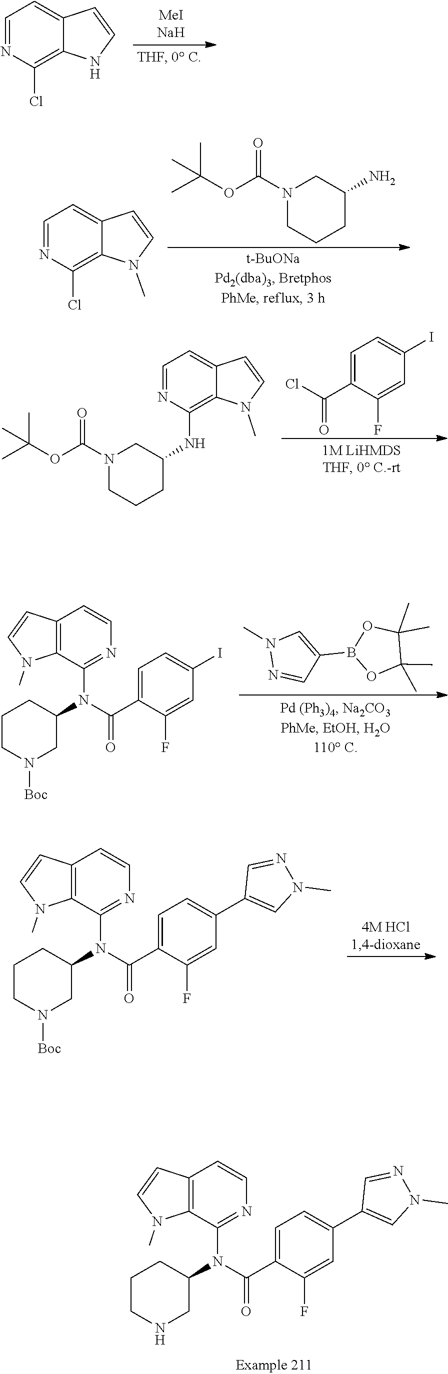

To a stirred solution of 7-chloro-1H-pyrrolo[2,3-c]pyridine (15 g, 98.30 mmol) in THF (150 mL) at 0° C. was added NaH (60% in mineral oil, 4.71 g, 117.9 mmol) in portion-wise. The reaction was then allowed to stir at 0° C. for 30 min and followed by addition of methyl iodide (13.4 ml, 216.2 mmol) was added dropwise. The reaction mixture was stirred at 0° C. for 1 h and then warmed to room temperature and stirred further for 2 h. The reaction mixture was diluted with water (300 mL) and extracted with EtOAc (2×500 mL). The combined organic layer was dried over Na₂SO₄ and concentrated under vacuum. The residue was purified by flash column chromatography on 60-120 mesh silica gel using 20% EtOAc in hexane to obtain 7-chloro-1-methyl-1H-pyrrolo[2,3-c]pyridine (14 g, 85%) as an off-white solid. LC-MS: [M+H⁺]=166.90.

### Step 2:

To a stirred solution of 7-chloro-1-methyl-1H-pyrrolo[2,3-c]pyridine (16 g, 96.03 mmol) and tert-butyl (R)-3-aminopiperidine-1-carboxylate (23 g, 115.23 mmol) in toluene (150 mL) at room temperature were added sodium tert-butoxide (18.4 g, 192.06 mmol), Bretphos (10.3 g, 19.20 mmol), and Pd₂(dba)₃ (8.79 g, 9.60 mmol). The reaction mixture was warmed to 110° C. and stirred for 3 h. The reaction mixture was diluted with water (300 mL) and extracted with EtOAc (3×500 mL). The combined organic layer was dried over Na₂SO₄ and concentrated. The residue was purified by flash column chromatography on silica gel (60-120 mesh) using 50% EtOAc in hexane to obtain tert-butyl (R)-3-((1-methyl-1H-pyrrolo[2,3-c]pyridin-7-yl)amino)piperidine-1-carboxylate (23 g, 72%). LC-MS: [M+H⁺]=331.25.

### Step 3:

To a stirred solution of tert-butyl (R)-3-((1-methyl-1H-pyrrolo[2,3-c]pyridin-7-yl)amino)piperidine-1-carboxylate (1.0 g, 3.026 mmol) in THF (10 mL) at 0° C. was added 1M LiHMDS in THF (9.07 mL, 9.07 mmol). The reaction was stirred for 30 min at 0° C. and followed by addition of a solution of 2-fluoro-4-iodobenzoyl chloride (1.3 g, 4.54 mmol, 1.5 eq.) in THF (10 mL). The reaction mixture was allowed to warm to room temperature and stir for 10 min. After completion of the reaction, the reaction mixture quenched with a saturated aq. NH₄Cl solution and extracted with ethyl acetate (2×30 mL). The combined organic layer was washed with brine (10 mL), dried over Na₂SO₄, and concentrated under reduced pressure. The residue was purified by Combi-flash using 20% EtOAc in hexane to afford tert-butyl (R)-3-(2-fluoro-4-iodo-N-(1-methyl-1H-pyrrolo[2,3-c]pyridin-7-yl)benzamido)piperidine-1-carboxylate (600 mg, 29%) as an off-white solid. LC-MS: [M+H⁺]=579.2.

### Step 4:

To a stirred solution of tert-butyl (R)-3-(2-fluoro-4-iodo-N-(1-methyl-1H-pyrrolo[2,3-c]pyridin-7-yl)benzamido)piperidine-1-carboxylate (200 mg, 0.345 mmol) in toluene (2 mL) and EtOH (2 mL) was added 1-methyl-4-(4,4,5,5-tetramethyl-1,3,2-dioxaborolan-2-yl)-1H-pyrazole (143.88 mg, 0.691 mmol) and followed by a solution of Na₂CO₃ (146.2 mg, 1.38 mmol) in H₂O (0.2 mL). The reaction mixture was degassed with nitrogen for 10 min and followed by addition of Pd(PPh₃) 4 (19.92 mg, 0.0127 mmol). The resulting reaction mixture was degassed again with nitrogen for 10 min and then stirred at 110° C. for 16 h. After completion of the reaction, the reaction mixture was diluted with water and extracted with ethyl acetate (2×30 mL). The combined organic layer was washed with brine (10 mL), dried over Na₂SO₄, and concentrated under reduced pressure. The residue was purified by Combi-flash using 70% EtOAc in hexane to afford tert-butyl (R)-3-(2-fluoro-4-(1-methyl-1H-pyrazol-4-yl)-N-(1-methyl-1H-pyrrolo[2,3-c]pyridin-7-yl)benzamido)piperidine-1-carboxylate (100 mg, 54%) as an off-white solid. LC-MS: [M+H⁺]=533.3.

### Step 5:

To a stirred solution of tert-butyl (R)-3-(2-fluoro-4-(1-methyl-1H-pyrazol-4-yl)-N-(1-methyl-1H-pyrrolo[2,3-c]pyridin-7-yl)benzamido)piperidine-1-carboxylate (100 mg, 187 mmol, 1.0 eq.) in DCM (2 mL) at 0° C. was added 4M HCl in 1,4-dioxane (1 mL). The reaction mixture was stirred at 25° C. for 2 h. The reaction mixture was concentrated under reduced pressure, and the crude compound was purified by Prep-HPLC (Conditions: Column: X-BRIDGE PREP OBD C18 (250*19) mm, 5 μm), Mobile phase A: 0.1% formic acid in water, Mobile phase B: 100% MeCN, Gradient: T/% B: 0/25, 10/45, Flow: 15 mL/min, Dilution: MeCN+H₂O+THF). Pure fractions were dried by lyophilization to afford (R)-2-fluoro-4-(1-methyl-1H-pyrazol-4-yl)-N-(1-methyl-1H-pyrrolo[2,3-c]pyridin-7-yl)-N-(piperidin-3-yl)benzamide formic acid salt (41 mg, 58%) as a white solid. LC-MS: [M+H⁺]=433.7. ¹H NMR (CD₃OD, 400 MHZ) δ 8.12 (d, J=5.6 Hz, 1H), 7.89-7.79 (m, 3H), 7.70 (s, 1H), 7.25 (t, J=7.6 Hz, 1H), 7.11-7.08 (m, 2H), 6.70 (d, J=2.0 Hz, 1H), 5.02-4.93 (m, 1H), 4.08-3.98 (m, 1H), 4.04 (s, 3H), 3.82 (s, 3H), 3.70-3.65 (m, 1H), 3.37-3.26 (m, 1H), 2.97-2.90 (m, 1H), 1.99-1.79 (m, 3H), 1.42-1.25 (m, 1H).

### Synthesis of (R)-2-fluoro-N-(1-methyl-1H-pyrrolo[2,3-c]pyridin-7-yl)-4-(3-methylisoxazol-5-yl)-N-(piperidin-3-yl)benzamide (Example 212)

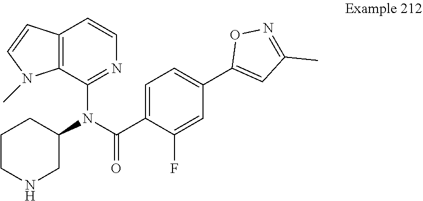 [SMILES: Cc1cc(-c2ccc(C(=O)N(c3nccc4ccn(C)c34)[C@@H]3CCCNC3)c(F)c2)on1]

Example 212 was prepared according to the procedure described for Example 211 by substituting 1-methyl-4-(4,4,5,5-tetramethyl-1,3,2-dioxaborolan-2-yl)-1H-pyrazole with 3-methyl-5-(4,4,5,5-tetramethyl-1,3,2-dioxaborolan-2-yl)isoxazole, provided the title compound hydrochloride salt as a white solid. LC-MS: [M+H⁺]=434.7. ¹H NMR (CD₃OD, 400 MHZ): δ 8.01 (d, J=5.6 Hz, 1H), 7.65-7.60 (m, 1H), 7.53-7.51 (m, 1H), 7.29-7.27 (m, 1H), 7.22-7.19 (m, 2H), 6.56-7.52 (m, 2H), 4.96-4.83 (m, 1H), 4.12-4.10 (m, 1H), 4.03 (s, 3H), 3.95-3.98 (m, 1H), 3.60-3.55 (m, 1H), 2.88-2.86 (m, 1H), 2.16 (s, 3H), 1.89-1.80 (m, 3H), 1.27-1.19 (m, 1H).

### Synthesis of (R)-2-(4-(3H-[1,2,3]triazolo[4,5-b]pyridin-3-yl)-2-fluoro-N-(piperidin-3-yl)benzamido)-N-methylnicotinamide (Example 213)

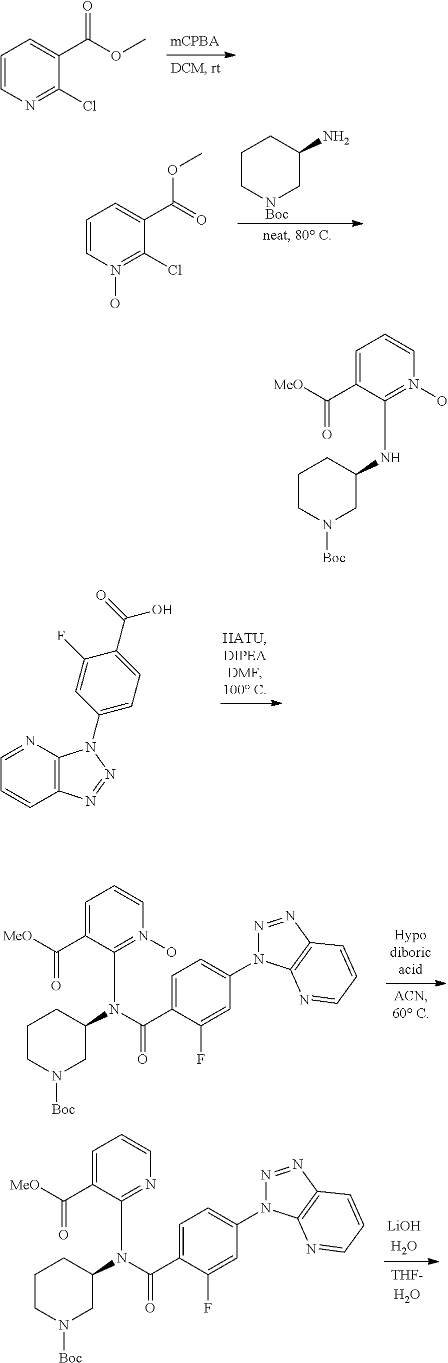

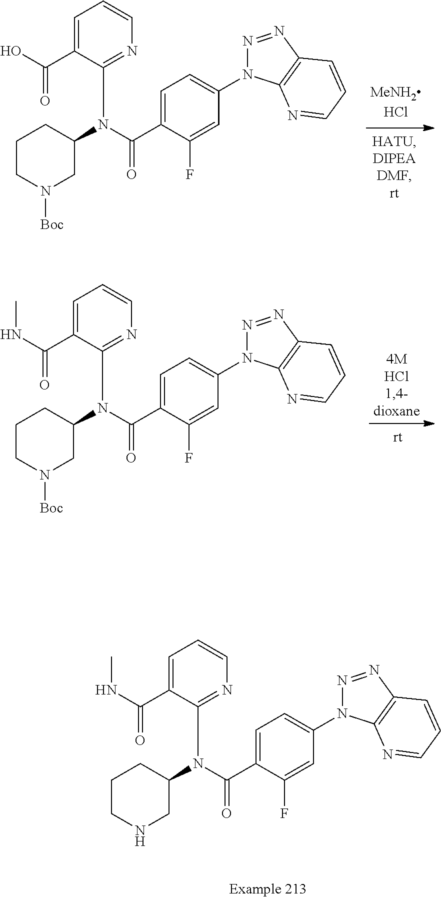 [SMILES: CC(C)(C)OC(=O)N1CCC[C@@H](N(C(=O)c2ccc(-n3nnc4cccnc43)cc2F)c2ncccc2C(=O)O)C1 ; CNC(=O)c1cccnc1N(C(=O)c1ccc(-n2nnc3cccnc32)cc1F)[C@@H]1CCCN(C(=O)OC(C)(C)C)C1 ; CNC(=O)c1cccnc1N(C(=O)c1ccc(-n2nnc3cccnc32)cc1F)[C@@H]1CCCNC1]

### Step 1:

To a solution of methyl 2-chloronicotinate (2 g, 11.65 mmol) in DCM (30 mL) at 0° C. was added m-CPBA (12.03 g, 69.93 mmol). The reaction mixture stirred at room temperature for 16 h and then was concentrated under reduced pressure. The residue was purified by Combi-flash using 100% EtOAc to afford 2-chloro-3-(methoxycarbonyl)pyridine 1-oxide (1.7 g, 9.06 mmol, 78%) as an off-white solid. LC-MS: [M+H⁺]=190.

### Step 2:

A mixture of 2-chloro-3-(methoxycarbonyl)pyridine 1-oxide (1.6 g, 8.55 mmol) and tert-butyl (R)-3-aminopiperidine-1-carboxylate (3 mL) in sealed tube was stirred at 60° C. for 4 h. The reaction mixture was diluted with EtOAc (100 mL), washed with water (50 mL) and 1N aq. HCl (50 mL). The organic layer was dried over Na₂SO₄ and concentrated under reduced pressure to afford (R)-2-((1-(tert-butoxycarbonyl)piperidin-3-yl)amino)-3-(methoxycarbonyl)pyridine 1-oxide (2.9 g) as a dark brown liquid. LC-MS: [M+H⁺]=352.2.

### Step 3:

To a solution of 4-(3H-[1,2,3]triazolo[4,5-b]pyridin-3-yl)-2-fluorobenzoic acid (2.1 g, 8.20 mmol) in DMF (10 mL) at 0° C. was added DIPEA (0.5 mL, 24.6 mmol) and HATU (9.35 g, 24.6 mmol). The reaction was stirred for 15 min and followed by addition of (R)-2-((1-(tertbutoxycarbonyl)piperidin-3-yl)amino)-3-(methoxycarbonyl)pyridine 1-oxide (2.9 g, 8.20 mmol). The reaction was stirred at 25° C. for 4 h and then poured into H₂O. The precipitate was collected by filtration, washed with H₂O, and dried to afford (R)-2-(4-(3H-[1,2,3]triazolo[4,5-b]pyridin-3-yl)-N-(1-(tert-butoxycarbonyl)piperidin-3-yl)-2-fluorobenzamido)-3-(methoxycarbonyl)pyridine 1-oxide (4.5 g) as a yellow solid. LC-MS: [M+H⁺]=593.3.

### Step 4:

To a solution of (R)-2-(4-(3H-[1,2,3]triazolo[4,5-b]pyridin-3-yl)-N-(1-(tert-butoxycarbonyl)piperidin-3-yl)-2-fluorobenzamido)-3-(methoxycarbonyl)pyridine 1-oxide (4.5 g, 7.58 mmol) in MeCN (50 mL) at room temperature was added hypodiboric acid (2.02 g, 22.74 mmol). The reaction mixture stirred at 60° C. for 4 h and then concentrated. The residue was diluted with water (100 mL) and extracted with ethyl acetate (2×100 mL). The combined organic layer was dried over Na₂SO₄ and concentrated under reduced pressure. The residue was purified by Combi-flash using 60% EtOAc in hexane and was further purified by Prep-HPLC (Conditions: Column: Luna OMEGA C-18 (250×21.2×5 μm), Mobile phase A: 0.1% formic acid in water, Mobile phase B: MeCN (100%), Gradient: Time (min)/% B: 0/55, 10/85. Flow: 15 mL/min, Dilution: MeCN+H₂O+THF. Pure fractions were combined and dried by lyophilization to afford methyl (R)-2-(4-(3H-[1,2,3]triazolo[4,5-b]pyridin-3-yl)-N-(1-(tert-butoxycarbonyl)piperidin-3-yl)-2-fluorobenzamido)nicotinate (350 mg) as an off-white solid. LC-MS: [M+H⁺]=519.7.

### Step 5:

To a solution of methyl (R)-2-(4-(3H-[1,2,3]triazolo[4,5-b]pyridin-3-yl)-N-(1-(tertbutoxycarbonyl) piperidin-3-yl)-2-fluorobenzamido)nicotinate (300 mg, 0.521 mmol) in MeOH (5 mL) and THF (10 mL) at room temperature was added NaOH (104 mg, 2.60 mmol) and H₂O (5 mL). The reaction mixture was stirred at 80° C. for 4 h and then concentrated under reduced pressure. The residue was diluted with water, acidified to pH 2 with 1N aq. HCl, and extracted with EtOAc (100 mL). The organic layer was dried by Na₂SO₄ and concentrated under reduced pressure to afford (R)-2-(4-(3H-[1,2,3]triazolo[4,5-b]pyridin-3-yl)-N-(1-(tert-butoxycarbonyl)piperidin-3-yl)-2-fluorobenzamido) nicotinic acid (230 mg, 78%) as an off-white solid. LC-MS: [M+H⁺]=562.3.

### Step 6:

To a solution of (R)-2-(4-(3H-[1,2,3]triazolo[4,5-b]pyridin-3-yl)-N-(1-(tert-butoxycarbonyl)piperidin-3-yl)-2-fluorobenzamido) nicotinic acid (50 mg, 0.089 mmol) in DMF (2 mL) at 0° C. was added DIPEA (0.1 mL, 0.267 mmol) and HATU (101 mg, 0.267 mmol). The reaction mixture stirred for 15 min and followed by addition of methylamine HCl (8.97 mg, 0.133 mmol). The reaction mixture was stirred at room temperature for 4 h. The reaction mixture was diluted with water and extracted with EtOAc (2×50 mL). The combined organic layer was washed with brine, dried over Na₂SO₄, and concentrated under reduced pressure to afford (140 mg). The residue was purified by Prep-HPLC (Conditions: Column: X-SELECT CSH C18 (250*19) mm, 5 μm), Mobile phase A: 0.1% formic acid in water, Mobile phase B: MeCN (100%), Gradient: Time (min)/% B: 0/40, 10/70. Flow: 12 mL/min, Dilution: MeCN+H₂O+THF). Pure fractions were combined and dried by lyophilization to afford tert-butyl (R)-3-(4-(3H-[1,2,3]triazolo[4,5-b]pyridin-3-yl)-2-fluoro-N-(3-(methylcarbamoyl)pyridin-2-yl)benzamido)piperidine-1-carboxylate (80 mg) as an off-white solid. LC-MS: [M+H⁺]=573.2.

### Step 7:

To a solution of tert-butyl (R)-3-(4-(3H-[1,2,3]triazolo[4,5-b]pyridin-3-yl)-2-fluoro-N-(3-(methylcarbamoyl)pyridin-2-yl)benzamido)piperidine-1-carboxylate (80 mg, 0.139 mmol) in DCM (2 mL) at 0° C. was added 4M HCl in 1,4-dioxane (5 mL). The reaction mixture was stirred at room temperature for 2 h and then concentrated under reduced pressure. The residue was purified by Prep-HPLC (Conditions: Column: X-SELECT CSH C18 (250*19) mm, 5 μm), Mobile phase A: 0.1% formic acid in water, Mobile phase B: MeCN (100%), Gradient: Time (min)/% B: 0/25, 2/25, 10/50. Flow: 12 mL/min, Dilution: MeCN+H₂O+THF). Pure fractions were combined and dried by lyophilization to afford (R)-2-(4-(3H-[1,2,3]triazolo[4,5-b]pyridin-3-yl)-2-fluoro-N-(piperidin-3-yl)benzamido)-N-methylnicotinamide formic acid salt (35 mg) as an off-white solid. LC-MS: [M+H⁺]=475.95. ¹H NMR (CD₃OD, 400 MHZ): δ 8.73 (d, J=4.0 Hz, 1H), 8.58-8.57 (m, 1H), 8.48-8.44 (m, 1H), 8.09-8.02 (m, 2H), 7.68 (d, J=7.6 Hz, 1H), 7.50-7.57 (m, 1H), 7.33-7.27 (m, 2H), 4.72-4.69 (m, 1H), 3.71-3.68 (m, 1H), 3.61-3.49 (m, 1H), 3.03 (m, 1H), 2.78 (m, 1H), 2.70 (s, 3H), 2.30 (m, 1H), 2.09-2.05 (m, 1H), 1.87-1.73 (m, 2H), 1.49-1.45 (m, 1H) 1.2 (sb, NH).

### Synthesis of (R)-2-fluoro-4-(1-methyl-1H-pyrazol-4-yl)-N-(8-methylisoquinolin-1-yl)-N-(piperidin-3-yl)benzamide (Example 214)

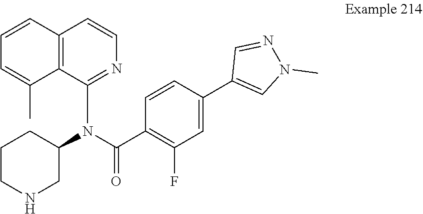 [SMILES: Cc1cccc2ccnc(N(C(=O)c3ccc(-c4cnn(C)c4)cc3F)[C@@H]3CCCNC3)c12]

Example 214 was prepared according to the procedure for Example 126 by substituting (1-methyl-5-phenyl-1H-pyrazol-4-yl)boronic acid with 1-methyl-4-(4,4,5,5-tetramethyl-1,3,2-dioxaborolan-2-yl)-1H-pyrazole to afford the title compound hydrochloride salt (108 mg) as an off-white solid. LC-MS: [M+H⁺]=444.35. ¹H NMR (CD₃OD, 400 MHz) δ 8.34 (d, J=5.6 Hz, 1H), 7.91 (bs, 1H), 7.77 (s, 1H), 7.67 (d, J=5.6 Hz, 1H), 7.58-7.53 (m, 1H), 7.44-7.35 (m, 2H), 6.96-6.89 (m, 1H), 6.82-6.80 (m, 1H), 6.73-6.68 (m, 1H), 5.00-4.86 (m, 1H), 3.92-3.88 (m, 1H), 3.80 (s, 3H), 3.75-3.72 (m, 1H), 3.24-3.29 (m, 1H), 2.93-2.89 (m, 1H), 2.73 (s, 3H), 2.61-2.57 (m, 1H), 1.94-1.81 (m, 2H), 1.51-1.44 (m, 1H).

### Synthesis of (R)-2-fluoro-N-(8-methylisoquinolin-1-yl)-N-(piperidin-3-yl)-4-(pyrazolo[1,5-a]pyrimidin-3-yl)benzamide (Example 215)

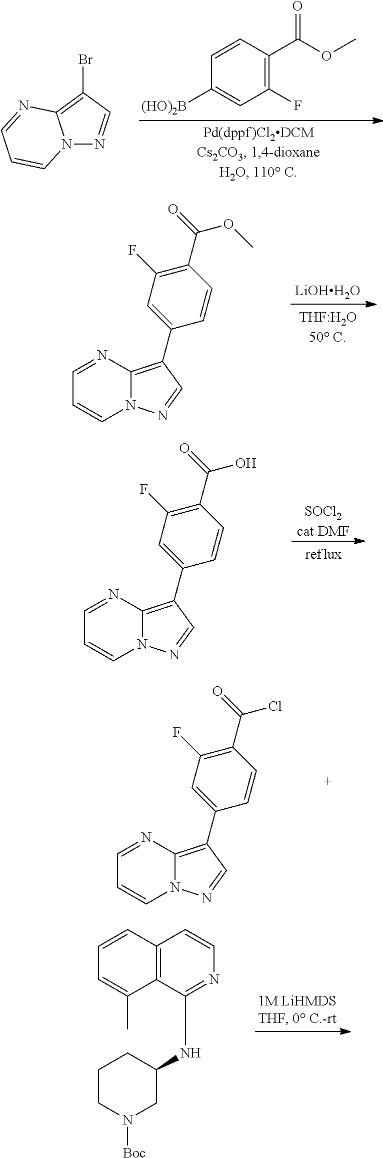

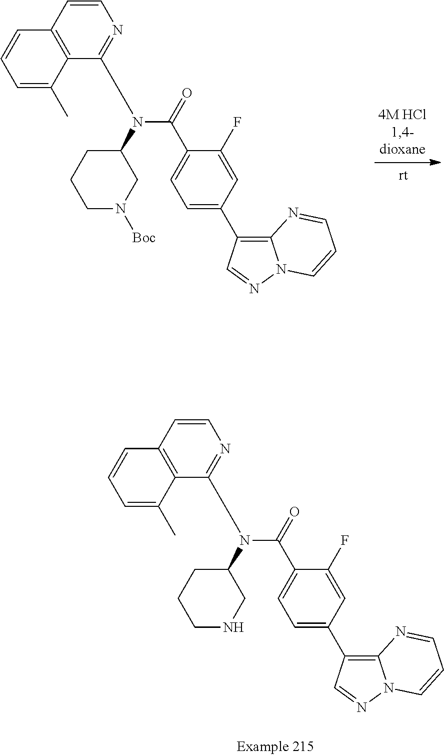 [SMILES: Cc1cccc2ccnc(N(C(=O)c3ccc(-c4cnn5cccnc45)cc3F)[C@@H]3CCCN(C(=O)OC(C)(C)C)C3)c12 ; Cc1cccc2ccnc(N(C(=O)c3ccc(-c4cnn5cccnc45)cc3F)[C@@H]3CCCNC3)c12]

### Step 1:

To a solution of 3-bromopyrazolo[1,5-a]pyrimidine (1 g, 5.049 mmol) and (4-(ethoxycarbonyl)-3-fluorophenyl)boronic acid (1.2 g, 6.058 mmol) in 1,4-dioxane (20 mL) and water (1 mL) at room temperature was added Cs₂CO₃ (4.93 g, 15.147 mmol) and Pd(dppf)Cl₂ (412 mg, 0.504 mmol). The reaction mixture was purged with nitrogen for 10 min and then stirred at 110° C. under nitrogen atmosphere for 16 h. The reaction mixture was diluted with water (50 mL) and extracted with EtOAc (3×50 mL). The combined organic layer was washed with brine, dried over Na₂SO₄, and concentrated under reduced pressure. The residue was purified by Combi-flash using eluted with 30-35% EtOAc in hexane to affords methyl 2-fluoro-4-(pyrazolo[1,5-a]pyrimidin-3-yl)benzoate (450 mg). LC-MS: [M+H⁺]=272.1.

### Step 2:

To a solution of methyl 2-fluoro-4-(pyrazolo[1,5-a]pyrimidin-3-yl)benzoate (450 mg, 1.628 mmol) in THF (10 mL) and H₂O (3 mL) at room temperature was added LiOH·H₂O (139 mg, 3.317 mmol). The reaction mixture was stirred for 16 h and then concentrated under reduced pressure. The residue was diluted with water and acidified to pH 2 with 1N aq. HCl. The precipitate was collected and dried to afford crude 2-fluoro-4-(pyrazolo[1,5-a]pyrimidin-3-yl)benzoic acid (300 mg).

### Step 3:

To a solution of 2-fluoro-4-(pyrazolo[1,5-a]pyrimidin-3-yl)benzoic acid (150 mg, 0.58 mmol) in SOCl₂ (10 mL) at room temperature was added a catalytic amount of DMF (0.05 mL). The reaction mixture was stirred at 60° C. for 16 h. The reaction mixture was allowed to cool and concentrated under reduced pressure and co-distilled with toluene to afford crude 2-fluoro-4-(pyrazolo[1,5-a]pyrimidin-3-yl)benzoyl chloride (140 mg) as a light brown solid.

### Step 4:

To a solution of tert-butyl (R)-3-((8-methylisoquinolin-1-yl)amino)piperidine-1-carboxylate (100 mg, 0.29 mmol) in THF (5 mL) at 0° C. was added 1M LiHMDS in THF (0.9 mL, 0.87 mmol). The reaction was stirred for 20 min and followed by addition of 2-fluoro-4-(pyrazolo[1,5-a]pyrimidin-3-yl)benzoyl chloride (128 mg, 0.46 mmol) in THF (5 mL). The reaction mixture stirred for 10 min and was quenched with saturated aq. NH₄Cl solution (15 mL) and extracted with ethyl acetate (2×40 mL). The combined organic layer was washed with brine, dried over Na₂SO₄, and concentrated under reduced pressure. The residue was purified by Prep-HPLC (Conditions: Luna OMEGA C-18 (250×21.2×5 μm), Mobile phase A: 0.1% ammonium acetate in water, Mobile phase B: 100% MeCN, Gradient (Time/(min)/% B): 0/70, 10/95, Flow: 15 mL/min). Pure fractions were combined and dried by lyophilization to afford the tert-butyl (R)-3-(2-fluoro-N-(8-methylisoquinolin-1-yl)-4-(pyrazolo[1,5-a]pyrimidin-3-yl)benzamido)piperidine-1-carboxylate (15 mg, 30%) a LC-MS: [M+H⁺]=581.8.

### Step 5:

The title compound hydrochloride salt was obtained as an off-white solid by deprotection Method A used in Example 97. LC-MS: [M+H⁺]=480.8. ¹H NMR (CD₃OD, 400 MHZ) δ 8.78 (dd, J=6.8 Hz, 1.6 Hz, 1H), 8.49 (dd, J=4.0 Hz, 1.6 Hz, 1H), 8.34 (d, J=5.2 Hz, 1H), 8.32 (s, 1H), 7.64-7.59 (m, 2H), 7.55-7.52 (m, 1H), 7.40-7.32 (m, 3H), 6.93 (dd, J=7.2 Hz, 4.0 Hz, 1H), 6.73-6.68 (m, 1H), 5.02-4.95 (m, 1H), 3.92-3.88 (m, 1H), 3.76-3.70 (m, 1H), 3.38-3.29 (m, 1H), 2.95-2.90 (m, 1H), 2.64-2.59 (m, 1H), 1.87-1.82 (m, 2H), 1.50-1.46 (m, 1H).

### Synthesis of (R)-4-(5-ethyl-3H-[1,2,3]triazolo[4,5-b]pyridin-3-yl)-2-fluoro-N-(8-methylisoquinolin-1-yl)-N-(piperidin-3-yl)benzamide (Example 216)

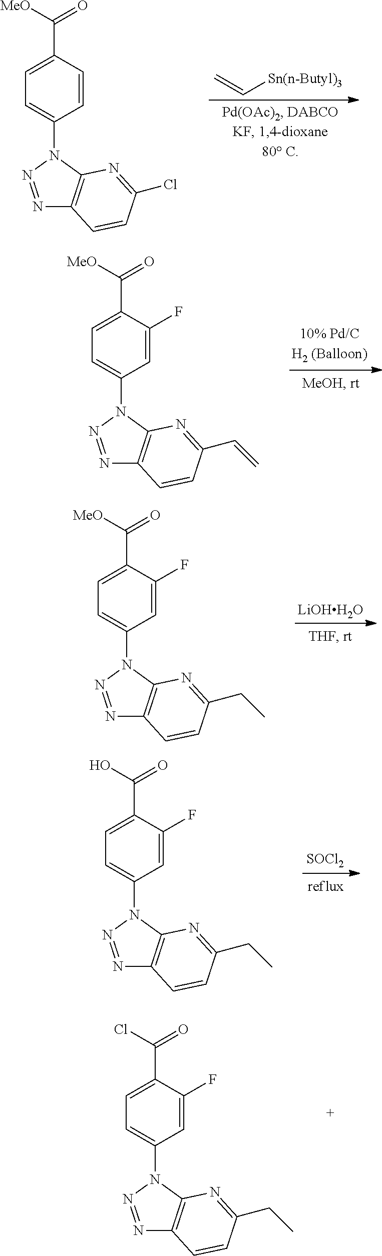

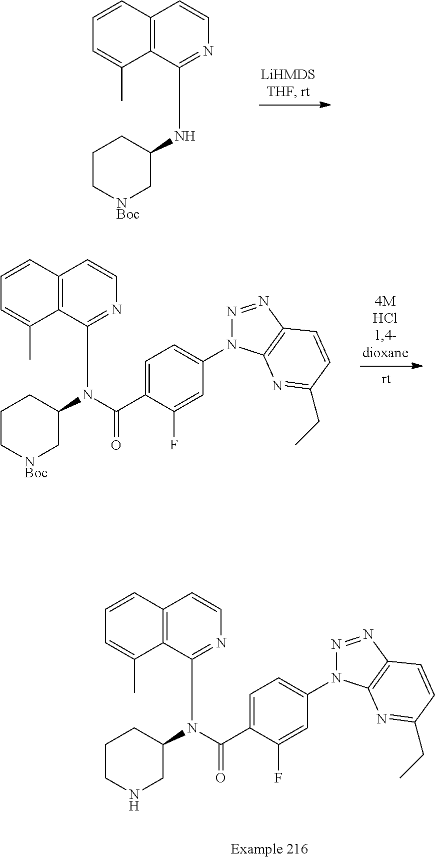 [SMILES: Cc1cccc2ccnc(N[C@@H]3CCCN(C(=O)OC(C)(C)C)C3)c12 ; CCc1ccc2nnn(-c3ccc(C(=O)N(c4nccc5cccc(C)c45)[C@@H]4CCCN(C(=O)OC(C)(C)C)C4)c(F)c3)c2n1 ; CCc1ccc2nnn(-c3ccc(C(=O)N(c4nccc5cccc(C)c45)[C@@H]4CCCNC4)c(F)c3)c2n1]

### Step 1:

To a solution of methyl 4-(5-chloro-3H-[1,2,3]triazolo[4,5-b]pyridin-3-yl)-2-fluorobenzoate (1.5 g, 4.897 mmol) in 1,4-dioxane (20 mL) at room temperature was added tributyl(vinyl)stannane (3.1 g, 9.794 mmol), potassium fluoride (850 mg, 14.691 mmol), and DABCO (1.6 g, 14.691 mmol). The reaction mixture was degassed with N₂ for 15 min and followed by addition of palladium(II) acetate (109 mg, 0.489 mmol) and then stirred at 90° C. for 16 h. The reaction mixture diluted with EtOAc (100 mL), filtered through celite and the filtrate was washed with water. The organic layer was washed with brine, dried over Na₂SO₄, and concentrated under reduced pressure. The residue was purified by Combi-flash using 80% EtOAc in hexane to afford methyl 2-fluoro-4-(5-vinyl-3H-[1,2,3]triazolo[4,5-b]pyridin-3-yl)benzoate (250 mg) as an off-white solid. LC-MS: [M+H⁺]=299.

### Step 2:

To a solution of methyl 2-fluoro-4-(5-vinyl-3H-[1,2,3]triazolo[4,5-b]pyridin-3-yl)benzoate (250 mg, 0.838 mmol) in MeOH (10 mL) at room temperature was added Pd/C (10 wt %, 500 mg) and the reaction was stirred under an atmosphere of hydrogen (50 psi) for 3 h. The reaction mixture was diluted with DCM, filtered through celite, rinsed with 10% MeOH in DCM. The filtrate was concentrated under reduced pressure to afford methyl 4-(5-ethyl-3H-[1,2,3]triazolo[4,5-b]pyridin-3-yl)-2-fluorobenzoate (210 mg) as a white solid. LC-MS: [M+H⁺]=301.

### Step 3:

To a solution of methyl 4-(5-ethyl-3H-[1,2,3]triazolo[4,5-b]pyridin-3-yl)-2-fluorobenzoate (210 mg, 1.79 mmol) in THF (5 mL) was added LiOH·H₂O (84 mg, 3.49 mmol) and H₂O (5 mL) at 0° C. and was then stirred at room temperature for 2 h. The reaction mixture was concentrated under reduced pressure. The residue was diluted with water and acidified to pH 2 with 1N aq. HCl. The precipitate was collected by filtration and dried to afford 4-(5-ethyl-3H-[1,2,3]triazolo[4,5-b]pyridin-3-yl)-2-fluorobenzoic acid (190 mg) as an off-white solid. LC-MS: [M+H⁺]=287.1.

### Step 4:

To a solution of 4-(5-ethyl-3H-[1,2,3]triazolo[4,5-b]pyridin-3-yl)-2-fluorobenzoic acid (190 mg, 0.664 mmol) in SOCl₂ (10 mL) was added a catalytic amount of DMF (0.1 mL) at 0° C. and the reaction mixture stirred at room temperature for 16 h. The reaction mixture was concentrated under reduced pressure and co-distilled with toluene to afford 4-(5-ethyl-3H-[1,2,3]triazolo[4,5-b]pyridin-3-yl)-2-fluorobenzoyl chloride (250 mg) as a yellow solid.

### Step 5:

To a solution of tert-butyl (R)-3-((8-methylisoquinolin-1-yl)amino)piperidine-1-carboxylate (100 mg, 0.293 mmol) in THF (10 mL) was added 1.5M LiHMDS in THF (0.5 mL, 0.879 mmol) at 0° C. and stirred for 10 min and followed by addition of 4-(5-chloro-3H-[1,2,3]triazolo[4,5-b]pyridin-3-yl)-2-fluorobenzoyl chloride (220 mg, 0.879 mmol) in THF (5 mL). The reaction mixture was stirred for 30 min before it was diluted with water (10 mL) and extracted with EtOAc (2×50 mL). The combined organic layer was washed with brine, dried over Na₂SO₄, and concentrated under reduced pressure. The residue was purified by Combi-flash using 80% EtOAc in hexane. The residue was further purified by Prep-HPLC (Conditions: Column: Luna OMEGA C-18 (250×21.2×5 μm), Mobile phase A: 0.1% formic acid in water, Mobile phase B: MeCN (100%), Gradient: Time (min)/% B: 0/65, 10/95, Flow: 15 mL/min, Dilution: MeCN+H₂O+THF). Pure fractions were combined and dried by lyophilization to afford tert-butyl (R)-3-(4-(5-ethyl-3H-[1,2,3]triazolo[4,5-b]pyridin-3-yl)-2-fluoro-N-(8-methylisoquinolin-1-yl)benzamido)piperidine-1-carboxylate (25 mg) as an off white solid. LC-MS: [M+H⁺]=610.

### Step 6:

To a solution of tert-butyl (R)-3-(4-(5-ethyl-3H-[1,2,3]triazolo[4,5-b]pyridin-3-yl)-2-fluoro-N-(8-methylisoquinolin-1-yl)benzamido)piperidine-1-carboxylate (24 mg, 0.032 mmol) in DCM (2 mL) at 0° C. was added 4M HCl in 1,4-dioxane (5 mL). The reaction was stirred at room temperature for 2 h and then concentrated under reduced pressure. The residue was purified by Prep-HPLC (Conditions: Column: SUNFIRE OBD PREP C-18 (250×19×5 μm), Mobile phase A: 0.1% formic acid in water, Mobile phase B: MeCN (100%), Gradient: Time (min)/% B: 0/30, 5/50, 10/100. Flow: 15 mL/min, Dilution: MeCN+H₂O+THF). Pure fractions were dried by lyophilization to afford (R)-4-(5-ethyl-3H-[1,2,3]triazolo[4,5-b]pyridin-3-yl)-2-fluoro-N-(8-methylisoquinolin-1-yl)-N-(piperidin-3-yl)benzamide formic acid salt (6 mg) as a white solid. LC-MS: [M+H⁺]=510. ¹H NMR (CD₃OD, 400 MHZ): δ 8.46 (d, J=5.6 Hz, 1H), 8.37 (d, J=8.4 Hz, 1H), 8.04 (dd, J=1.6, 11.2 Hz, 1H), 7.88-7.82 (m, 1H), 7.75 (d, J=5.2 Hz, 1H), 7.66-7.64 (m, 1H), 7.51 (d, J=4.8 Hz, 2H), 7.43 (d, J=8.8 Hz, 1H), 7.16-7.07 (m, 1H), 5.08 (m, 1H), 3.99-3.98 (m, 1H), 3.90-3.87 (m, 1H), 3.83-3.77 (m, 1H), 3.52-3.50 (m, 1H), 3.46-3.36 (m, 1H), 3.02 (q, J=7.6 Hz, 2H), 2.87 (s, 3H), 1.97-1.94 (m, 3H), 1.35 (t, J=7.6 Hz, 3H).

### Synthesis of tert-butyl (R)-3-(2-fluoro-4-(5-methyl-3H-[1,2,3]triazolo[4,5-b]pyridin-3-yl)-N-(8-methylisoquinolin-1-yl)benzamido)piperidine-1-carboxylate (Example 217)

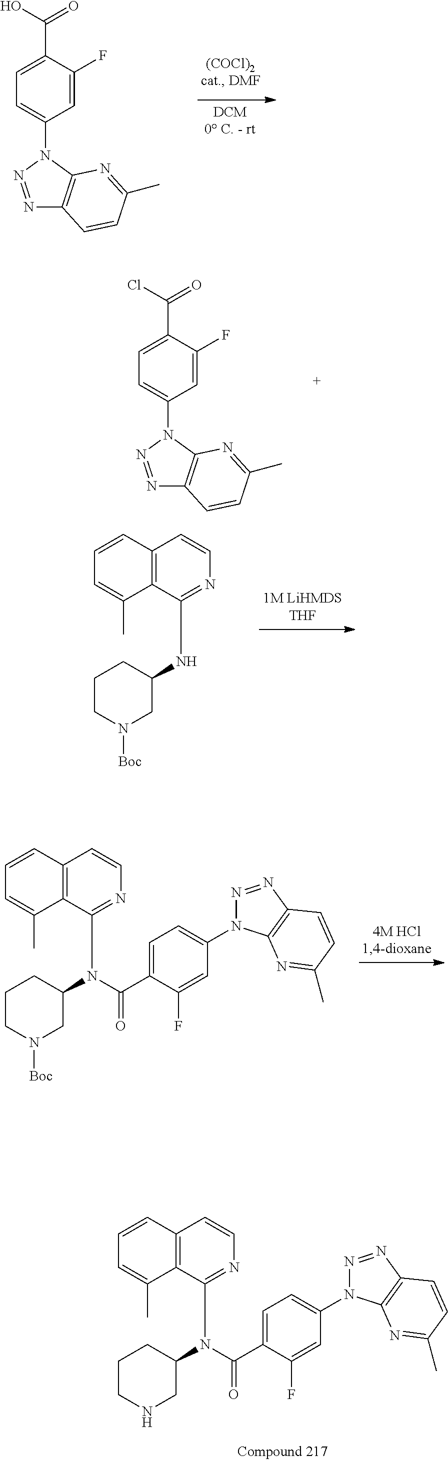

### Step 1:

To a stirred solution of 2-fluoro-4-(5-methyl-3H-[1,2,3]triazolo[4,5-b]pyridin-3-yl)benzoic acid (300 mg, 1.102 mmol) in DCM (10 mL) at 0° C. were added oxalyl chloride (0.2 mL, 1.654 mmol) and DMF (0.1 mL). The reaction mixture was warmed to room temperature and stirred for 2 h. The reaction mixture was then concentrated under reduced pressure to afford crude 2-fluoro-4-(5-methyl-3H-[1,2,3]triazolo[4,5-b]pyridin-3-yl)benzoyl chloride (300 mg) as a solid.

### Step 2:

To a stirred solution of tert-butyl (R)-3-((8-methylisoquinolin-1-yl)amino)piperidine-1-carboxylate (250 mg, 0.732 mmol) in THF (6 mL) at 0° C. was added 1M LiHMDS in THF (1.4 mL, 1.4 mmol). The reaction was stirred for 20 min and followed by addition of 2-fluoro-4-(5-methyl-3H-[1,2,3]triazolo[4,5-b]pyridin-3-yl)benzoyl chloride (212 mg, 0.732 mmol) in THF (5 mL). The reaction mixture was stirred at 0° C. for 10 min and then warmed to room temperature and stirred for 2 h. The reaction mixture was quenched with saturated aq. NH₄Cl (5 mL) and extracted with ethyl acetate (2×10 mL). The combined organic layer was washed with brine (10 mL), dried over Na₂SO₄, and concentrated under reduced pressure. The residue was purified by Combi-flash using 50% EtOAc in hexane. The residue was further purified by Prep-HPLC (Conditions: Column: YMC-ACTUS TRIART-EXRS (250*20) mm, 5 μm, Mobile phase A: 10 mM ammonium acetate in water, Mobile phase B: 100% MeCN, Gradient: T/% B: 0/65, 10/85, Flow: 15 mL/min, Dilution: MeCN+H₂O+THF). Pure fractions were dried by lyophilization to afford tert-butyl (R)-3-(2-fluoro-4-(5-methyl-3H-[1,2,3]triazolo[4,5-b]pyridin-3-yl)-N-(8-methylisoquinolin-1-yl)benzamido)piperidine-1-carboxylate (80 mg, 50%) as a light brown solid. LC-MS: [M+H⁺]=596.3.

### Step 3:

The title compound hydrochloride salt was obtained as an off-white solid by deprotection Method A used in Example 97. LC-MS: [M+H⁺]=496.7. ¹H NMR (DMSO-d₆ at 90° C., 400 MHZ) δ 9.23 (bs, 1H, NH), 8.47-8.42 (m, 2H), 7.90-7.88 (m, 1H), 7.77-7.68 (m, 3H), 7.57-7.52 (m, 2H), 7.42 (d, J=8.8 Hz, 1H), 7.15-7.11 (m, 1H), 5.02-4.97 (m, 1H), 3.86-3.84 (m, 1H), 3.64-3.59 (m, 1H), 3.30-3.24 (m, 1H), 2.95-2.81 (m, 1H), 2.84 (s, 3H), 2.66 (s, 3H), 1.92-1.84 (m, 3H), 1.42-1.39 (m, 1H).

### Synthesis of (R)-4-(3H-[1,2,3]triazolo[4,5-b]pyridin-3-yl)-2,6-difluoro-N-(8-methylisoquinolin-1-yl)-N-(piperidin-3-yl)benzamide (Example 218)

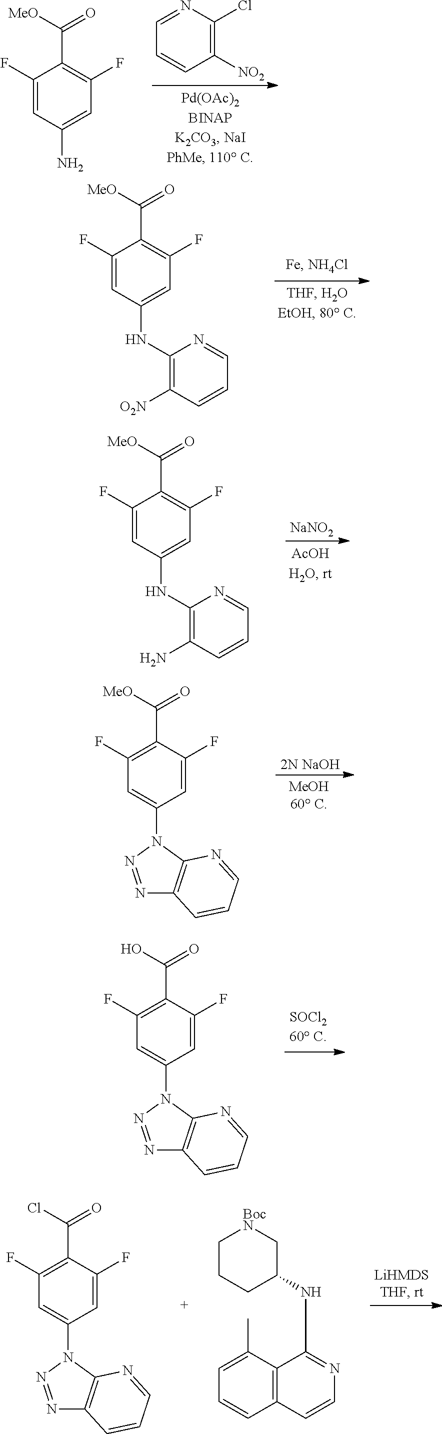

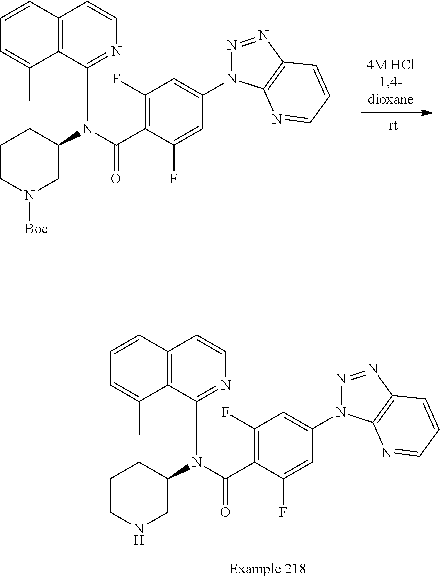 [SMILES: Cc1cccc2ccnc(N(C(=O)c3c(F)cc(-n4nnc5cccnc54)cc3F)[C@@H]3CCCN(C(=O)OC(C)(C)C)C3)c12 ; Cc1cccc2ccnc(N(C(=O)c3c(F)cc(-n4nnc5cccnc54)cc3F)[C@@H]3CCCNC3)c12]

### Step 1:

To a stirred solution of methyl 4-amino-2,6-difluorobenzoate (2.0 g, 10.68 mmol) in toluene (20 mL) was added 2-chloro-3-nitropyridine (1.69 g, 10.68 mmol), potassium carbonate (2.95 g, 21.37 mmol), and sodium iodide (48 mg, 0.320 mmol) respectively and degassed with nitrogen for 15 min and followed by addition of racemic BINAP (132 mg, 0.213 mmol) and palladium acetate (0.047 g, 0.213 mmol). The reaction mixture and again degassed for 10 min and then stirred at 110° C. for 16 h. The reaction mixture was cooled to room temperature and filtered through a pad of celite and rinsed with EtOAc. The filtrate was concentrated under reduced pressure. The residue was dissolved in EtOAc and washed with brine, dried over Na₂SO₄, and concentrated under reduced pressure. The residue was triturated with acetone, and the precipitate was collected and dried to afford methyl 2,6-difluoro-4-((3-nitropyridin-2-yl)amino)benzoate (2 g, 60%) as a yellow solid. LC-MS: [M+H⁺]=310.2.

### Step 2:

To a stirred solution of methyl 2,6-difluoro-4-((3-nitropyridin-2-yl)amino)benzoate (2.00 g, 6.46 mmol) in MeOH (20 mL), THF (20 mL) and water (20 mL) was added iron powder (1.80 g, 32.33 mmol, 5.0 eq.) and followed by ammonium chloride (2.76 g, 51.68 mmol) in one portion. The resulting reaction mixture was gradually warmed at 70° C. and stirred for 4 h. The reaction mixture was cooled to room temperature, filtered through a pad of celite, and rinsed with EtOAc. The filtrate was washed with water, brine, dried over Na₂SO₄, and concentrated under reduced pressure to afford crude methyl 4-((3-aminopyridin-2-yl)amino)-2,6-difluorobenzoate (1.7 g, 94%) as a brown solid. LC-MS: [M+H⁺]=280.1.

### Step 3:

To a stirred solution of methyl 4-((3-aminopyridin-2-yl)amino)-2,6-difluorobenzoate (1.7 g, 6.08 mmol, 1.0 eq.) in acetic acid (17 mL) and H₂O (17 mL) at 0° C. was added sodium nitrite (546 mg, 7.91 mmol) and the reaction was stirred at 25° C. for 4 h. The reaction mixture was diluted with water and extracted with DCM (120 mL). The organic layer was washed with brine, dried over Na₂SO₄, and concentrated under reduced pressure. The residue was triturated with diethyl ether to provide a solid which was dried to afford methyl 4-(3H-[1,2,3]triazolo[4,5-b]pyridin-3-yl)-2,6-difluorobenzoate (1.7 g, 96%) as a brown solid. LC-MS: [M+H⁺]=2 91.1.

### Step 4:

To a stirred solution of methyl 4-(3H-[1,2,3]triazolo[4,5-b]pyridin-3-yl)-2,6-difluorobenzoate (2 g, 6.89 mmol) in methanol (13 mL) at room temperature was added 2N aq. NaOH (25 mL) and the reaction mixture stirred at 60° C. for 4 h. The reaction mixture cooled to room temperature and concentrated under reduced pressure. The residue was dissolved in water and washed with diethyl ether. The aqueous layer was acidified to pH 2 with 1N aq. HCl and extracted with EtOAc (2×100 mL). The combined organic layer was dried over Na₂SO₄ and concentrated under reduced pressure to afford crude 4-(3H-[1,2,3]triazolo[4,5-b]pyridin-3-yl)-2,6-difluorobenzoic acid (1.4 g).

### Step 5:

To a stirred solution of 4-(3H-[1,2,3]triazolo[4,5-b]pyridin-3-yl)-2,6-difluorobenzoic acid (250 mg, 0.905 mmol) in thionyl chloride (3.75 mL) at 0° C. was dropwise added DMF (0.02 mL). The resulting reaction mixture stirred at 60° C. for 16 h. The reaction mixture was then concentrated under reduced pressure and co-distilled with toluene to afford crude 4-(3H-[1,2,3]triazolo[4,5-b]pyridin-3-yl)-2,6-difluorobenzoyl chloride (265 mg) as a pale brown solid.

### Step 6:

To a stirred solution of tert-butyl (R)-3-((8-methylisoquinolin-1-yl)amino)piperidine-1-carboxylate (268 mg, 0.784 mmol) in THF (3 mL) at 0° C. was added 1M LiHMDS in THF (2.35 mL, 1.09 mmol) dropwise. The reaction was stirred for 5 min and followed by addition of 4-(3H-[1,2,3]triazolo[4,5-b]pyridin-3-yl)-2,6-difluorobenzoyl chloride (300 mg, 1.019 mmol) in THF (1.5 mL). The reaction mixture and stirred for 15 min and then quenched with saturated aq. NH₄Cl (30 mL) and extracted with ethyl acetate (2×60 mL). The combined organic layer was washed with brine (10 mL), dried over Na₂SO₄, and concentrated under reduced pressure. The crude residue was purified by Prep-HPLC (Conditions: Column: ZORBAX BONUS-RP (150×21.2×5 μm), Mobile phase A: 0.1% formic acid in water, Mobile phase B: 100% MeCN, Gradient: Time (min)/% B: 0/65, 10/75, Flow: 15 mL/min, Dilution: MeCN+H₂O+THF). Pure fractions were combined and dried by lyophilization to afford tert-butyl (R)-3-(4-(3H-[1,2,3]triazolo[4,5-b]pyridin-3-yl)-2,6-difluoro-N-(8-methylisoquinolin-1-yl)benzamido)piperidine-1-carboxylate. LC-MS: [M+H⁺]=600.3.

### Step 7:

To a stirred solution of tert-butyl (R)-3-(4-(3H-[1,2,3]triazolo[4,5-b]pyridin-3-yl)-2,6-difluoro-N-(8-methylisoquinolin-1-yl)benzamido)piperidine-1-carboxylate (68 mg, 0.113 mmol,) in 1,4-dioxane (2 mL) at 0° C. was added 4M HCl in 1,4-dioxane (3 mL). The reaction was stirred at room temperature for 2 h and then concentrated under reduced pressure. The residue was purified by Prep-HPLC (Conditions: Column: ZORBAX BONUS-RP (150×21.2×5 μm), Mobile phase A: 0.1% formic acid in water, Mobile phase B: 100% MeCN, Gradient: Time (min)/% B: 0/25, 10/50, Flow: 15 mL/min, Dilution: MeCN+H₂O+THF). Pure fractions were combined and dried by lyophilization to afford (R)-4-(3H-[1,2,3]triazolo[4,5-b]pyridin-3-yl)-2,6-difluoro-N-(8-methylisoquinolin-1-yl)-N-(piperidin-3-yl)benzamide formic acid salt (24 mg) as an off-white solid. LC-MS: [M+H⁺]=500.55. ¹H NMR (DMSO-d₆ at 90° C., 400 MHZ): δ 8.67 (d, J=8.4 Hz, 1H), 8.41 (d, J=8.4 Hz, 1H), 8.27-8.24 (m, 1H), 7.70-7.50 (m, 4H), 7.43-7.31 (m, 3H), 5.02-4.95 (m, 1H), 3.98-3.95 (m, 1H), 3.80-3.74 (m, 1H), 3.39-3.31 (m, 1H), 2.99-2.94 (m, 1H), 2.82 (s, 3H), 2.62 (s, 1H), 1.98-1.85 (m, 3H), 1.65-1.60 (m, 1H).

### Synthesis of (R)—N-(8-methylisoquinolin-1-yl)-4-morpholino-N-(piperidin-3-yl)benzamide (Example 219)

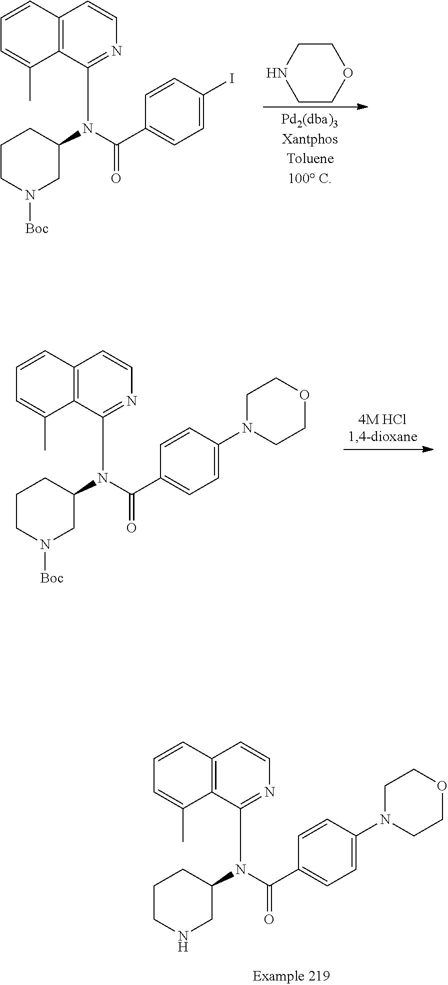 [SMILES: Cc1cccc2ccnc(N(C(=O)c3ccc(I)cc3)[C@@H]3CCCN(C(=O)OC(C)(C)C)C3)c12 ; C1COCCN1 ; Cc1cccc2ccnc(N(C(=O)c3ccc(N4CCOCC4)cc3)[C@@H]3CCCN(C(=O)OC(C)(C)C)C3)c12 ; Cc1cccc2ccnc(N(C(=O)c3ccc(N4CCOCC4)cc3)[C@@H]3CCCNC3)c12]

### Step 1:

To a stirred solution of tert-butyl (R)-3-(4-iodo-N-(8-methylisoquinolin-1-yl) benzamido) piperidine-1-carboxylate (80 mg, 0.14 mmol, 1.0 eq.) in toluene (5 ml) was added Cs₂CO₃ (136 mg, 0.42 mmol, 3.0 eq.), Xantphos (4 mg, 0.007 mmol, 0.05 eq.) and morpholine (0.03 ml, 0.42 mmol, 3.0 eq.). The resulting reaction mixture was purged with nitrogen gas for 10 min and followed by Pd₂(dba)₃ (6 mg, 0.007 mmol, 0.05 eq.). The reaction mixture and again degassed with nitrogen for 5 min and then stirred at 50° C. for 16 h. The reaction mixture was cooled to room temperature, diluted with water (10 mL), and extracted with ethyl acetate (2×80 mL). The combined organic layer was washed with brine (20 mL), dried over Na₂SO₄, and concentrated under reduced pressure. The residue was purified by Combi Flash using 35% EtOAc in hexane to afford tert-butyl (R)-3-(N-(8-methylisoquinolin-1-yl)-4-morpholinobenzamido)piperidine-1-carboxylate (65 mg, 87%) as a light brown solid. LC-MS: [M+H⁺]=531.3.

### Step 2:

The title compound hydrochloride salt was obtained as an off-white solid by deprotection Method A used in Example 97. LC-MS: [M+H⁺]=431.3. ¹H NMR (CD₃OD, 400 MHZ): δ 10.16-9.73 (m, 2H), 9.44-9.38 (m, 1H), 8.83-8.79 (m, 1H), 8.72 (d, J=7.6 Hz, 1H), 8.50-8.44 (m, 1H), 8.35-8.29 (m, 1H), 7.77 (d, J=8.8 Hz, 2H), 7.42 (d, J=8.8 Hz, 2H), 5.88-5.67 (m, 1H), 4.69-4.30 (m, 5H), 4.17-3.99 (m, 2H), 3.98-3.88 (m, 4H), 3.54 (s, 3H), 3.31-3.24 (m, 1H), 2.83-2.46 (m, 3H), 2.06-1.99 (m, 1H).

### Synthesis of (R)—N-(8-methylisoquinolin-1-yl)-N-(piperidin-3-yl)quinoline-7-carboxamide (Example 220)

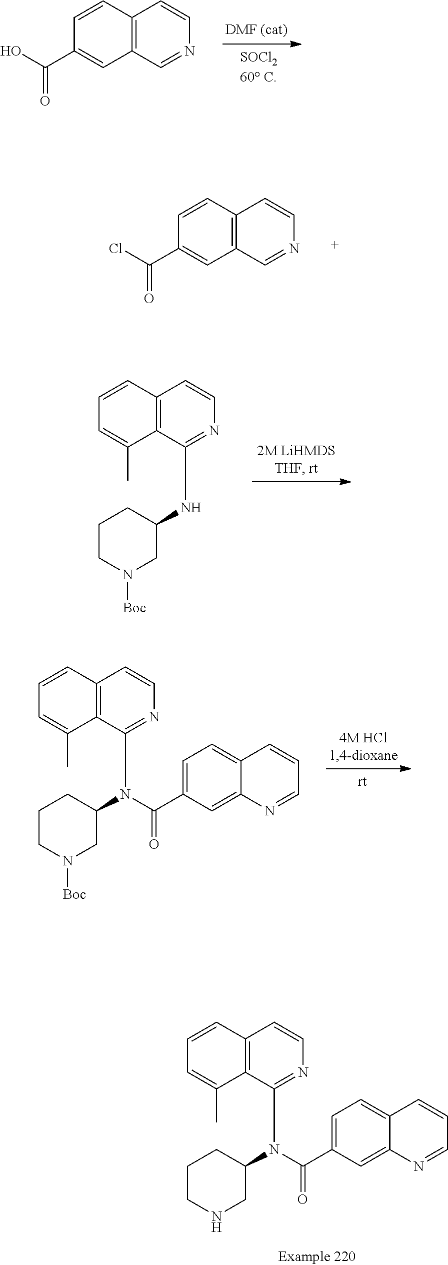 [SMILES: O=C(O)c1ccc2ccncc2c1 ; O=C(Cl)c1ccc2ccncc2c1 ; Cc1cccc2ccnc(N[C@@H]3CCCN(C(=O)OC(C)(C)C)C3)c12 ; Cc1cccc2ccnc(N(C(=O)c3ccc4cccnc4c3)[C@@H]3CCCN(C(=O)OC(C)(C)C)C3)c12 ; Cc1cccc2ccnc(N(C(=O)c3ccc4cccnc4c3)[C@@H]3CCCNC3)c12]

### Step 1:

To a stirred solution of quinoline-7-carboxylic acid (500 mg, 2.88 mmol) in SOCl₂ (10 mL) was added a catalytic amount of DMF (0.01 mL) at room temperature and stirred for 2 h. The reaction mixture was concentrated under reduced pressure and co-distilled with toluene to afford crude quinoline-7-carbonyl chloride (500 mg) as an off-white solid.

### Step 2:

To the stirred solution of tert-butyl (R)-3-((8-methylisoquinolin-1-yl)amino)piperidine-1-carboxylate (400 mg, 1.17 mmol) in THF (10 mL) at 0° C. was added 1M LiHMDS in THF (4 mL, 3.51 mmol). The reaction was stirred for 15 min and followed by addition of quinoline-7-carbonyl chloride (340 mg, 1.75 mmol) in THF (4 mL). The reaction mixture was stirred at room temperature for 15 min before it was diluted with water (30 mL) and extracted with ethyl acetate (2×80 mL). The combined organic layer was washed with brine (20 mL), dried over Na₂SO₄, and concentrated under reduced pressure. The residue was purified by Combi-Flash using 15-20% EtOAc in hexane first and then further purified by Prep-HPLC (Conditions: Column: X-BRIDGE PREP OBD C18 (250*19) mm, 5 μm, Mobile phase A: 0.1% ammonium acetate in water, Mobile phase B: 100% MeCN, Gradient: Time (min)/% B: 0/60, 10/80, Flow: 15 mL/min, Dilution: MeCN+H₂O+THF). The pure fractions were combined and dried by lyophilization to afford tert-butyl (R)-3-(N-(8-methylisoquinolin-1-yl)quinoline-7-carboxamido)piperidine-1-carboxylate.

### Step 3:

The title compound hydrochloride salt was obtained as an off-white solid by deprotection Method A used in Example 97. LC-MS: [M+H⁺]=397.75. ¹H NMR (CD₃OD, 400 MHZ) δ 9.05 (d, J=4.8 Hz, 1H), 8.91 (d, J=8.4 Hz, 1H), 8.48 (d, J=5.6 Hz, 1H), 7.95-7.90 (m, 2H), 7.82 (s, 1H), 7.70 (d, J=5.6 Hz, 1H), 7.61-7.50 (m, 2H), 7.35-7.30 (m, 2H), 5.09-5.01 (m, 1H), 3.97-3.94 (m, 1H), 3.79-3.73 (m, 1H), 3.41-3.30 (m, 1H), 2.94-2.91 (m, 1H), 2.74 (s, 3H), 2.65-2.61 (m, 1H), 1.91-1.77 (m, 2H), 1.44-1.37 (m, 1H).

### Synthesis of (R)-4-(3H-[1,2,3]triazolo[4,5-b]pyridin-3-yl)-2-fluoro-N-(piperidin-3-yl)-N-(8-(trifluoromethyl)isoquinolin-1-yl)benzamide (Example 221)

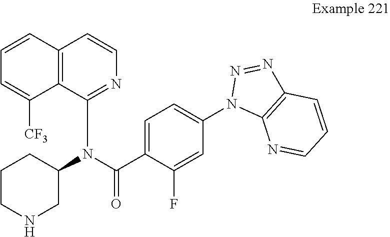 [SMILES: O=C(c1ccc(-n2nnc3cccnc32)cc1F)N(c1nccc2cccc(C(F)(F)F)c12)[C@@H]1CCCNC1]

Example 221 was synthesized following the procedure for Example 143 by substituting 6-bromo-1-chloroisoquinoline with 1-chloro-8-(trifluoromethyl) isoquinoline, provided the title compound hydrochloride salt as an off-white solid. LC-MS: [M+H⁺]=536.60. ¹H NMR (CD₃OD, 400 MHZ) 8.74 (d, J=4.4 Hz, 1H), 8.58-8.54 (m, 1H), 8.49 (d, J=8.4 Hz, 1H), 8.12-8.05 (m, 1H), 7.95-7.93 (m, 1H), 7.83-7.74 (m, 3H), 7.63-7.57 (m, 1H), 7.55-7.50 (m, 1H), 7.03-6.95 (m, 1H), 4.08-3.93 (m, 2H), 3.44-3.31 (m, 1H), 3.10-3.04 (m, 1H), 2.91-2.78 (m, 1H), 2.26-2.18 (m, 2H), 2.10-2.07 (m, 1H), 1.99-1.95 (m, 1H).

### Synthesis of (R)-4-(3H-[1,2,3]triazolo[4,5-b]pyridin-3-yl)-2-fluoro-N-(8-methoxyisoquinolin-1-yl)-N-(piperidin-3-yl)benzamide (Example 222)

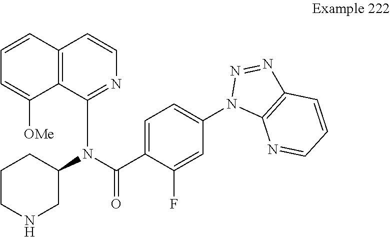 [SMILES: COc1cccc2ccnc(N(C(=O)c3ccc(-n4nnc5cccnc54)cc3F)[C@@H]3CCCNC3)c12]

Example 222 was synthesized following the procedure for Example 143 by substituting 6-bromo-1-chloroisoquinoline with 1-chloro-8-methoxyisoquinoline, provided the title compound as a hydrochloride salt as an off white solid. LC-MS: [M+H⁺]=498.3. ¹H NMR (DMSO-d₆ at 90° C., 400 MHZ) δ 8.78 (d, J=4.4 Hz, 1H), 8.60 (d, J=8.4 Hz, 1H), 8.42-8.40 (m, 1H), 7.87 (d, J=9.2 Hz, 1H), 7.78-7.76 (m, 1H), 7.70 (d, J=5.6 Hz, 1H), 5.59-7.54 (m, 2H), 7.38-7.36 (m, 1H), 7.23-7.11 (m, 2H), 5.05-4.98 (m, 1H), 4.00 (s, 3H), 3.73-3.70 (m, 1H), 3.55-3.48 (m, 2H), 2.85-2.78 (m, 1H), 1.86-1.81 (m, 3H), 1.34-1.29 (m, 1H).

### Synthesis of (R)-4-(1H-benzo[d][1,2,3]triazol-1-yl)-N-(8-methylisoquinolin-1-yl)-N-(piperidin-3-yl)benzamide (Example 223)

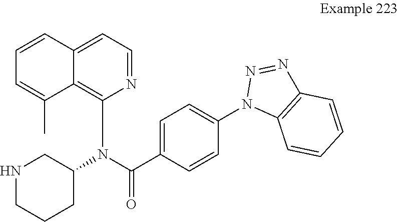 [SMILES: Cc1cccc2ccnc(N(C(=O)c3ccc(-n4nnc5ccccc54)cc3)[C@@H]3CCCNC3)c12]

Example 223 was prepared following the procedure for Example 218 by substituting methyl 4-amino-2,6-difluorobenzoate with methyl 4-aminobenzoate, provided the title compound hydrochloride salt as a white solid. LC-MS: [M+H⁺]=463.5. ¹H NMR (CD₃OD, 400 MHZ) 8.44-8.37 (m, 2H), 7.94 (d, J=8.4 Hz, 1H), 7.73 (d, J=5.6 Hz, 1H), 7.60-7.57 (m, 1H), 7.53-7.47 (m, 2H), 7.41-7.35 (m, 4H), 7.33-7.30 (m, 1H), 7.25-7.22 (m, 2H), 4.93-4.83 (m, 1H), 3.80-3.77 (m, 1H), 3.59-3.53 (m, 1H), 3.16-2.99 (m, 1H), 2.70 (s, 3H), 2.64-2.58 (m, 1H), 1.89-1.73 (m, 3H), 1.38-1.34 (m, 1H).

### Synthesis of 4-(3H-[1,2,3]triazolo[4,5-b]pyridin-3-yl)-N-(8-methylisoquinolin-1-yl)-N-((3R,4S)-4-methylpiperidin-3-yl)benzamide (Example 224)

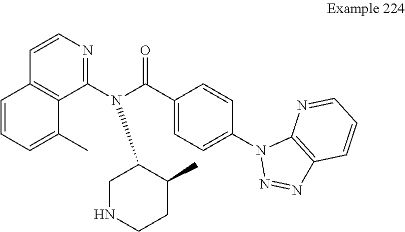 [SMILES: Cc1cccc2ccnc(N(C(=O)c3ccc(-n4nnc5cccnc54)cc3)[C@H]3CNCC[C@@H]3C)c12]

Example 224 was synthesized following the procedure for Example 50 by substituting tert-butyl (R)-3-aminopiperidine-1-carboxylate with tert-butyl (3R,4S)-3-amino-4-methylpiperidine-1-carboxylate, provided the title compound hydrochloride salt as a white solid. LC-MS: [M+H⁺]=478.0.

### Synthesis of 4-(3H-[1,2,3]triazolo[4,5-b]pyridin-3-yl)-N-(8-methylisoquinolin-1-yl)-N-((3R,5S)-5-methylpiperidin-3-yl)benzamide (Example 225)

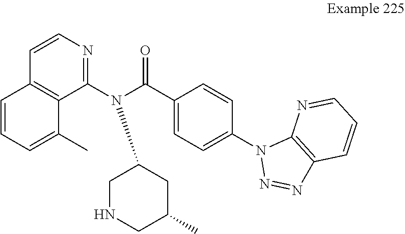 [SMILES: Cc1cccc2ccnc(N(C(=O)c3ccc(-n4nnc5cccnc54)cc3)[C@H]3CNC[C@@H](C)C3)c12]

Example 225 was synthesized following the procedure for Example 50 by substituting tert-butyl (R)-3-aminopiperidine-1-carboxylate with tert-butyl (3R,5S)-3-amino-5-methylpiperidine-1-carboxylate, provided the title compound hydrochloride salt as a white solid. LC-MS: [M+H⁺]=478.2.

### Synthesis of 4-(3H-[1,2,3]triazolo[4,5-b]pyridin-3-yl)-N-(8-methylisoquinolin-1-yl)-N-((3R,6R)-6-methylpiperidin-3-yl)benzamide (Example 226)

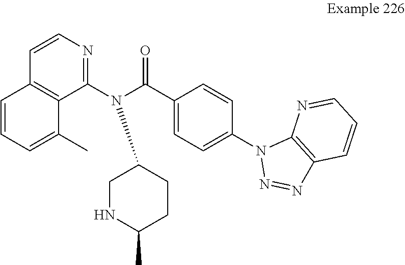 [SMILES: Cc1cccc2ccnc(N(C(=O)c3ccc(-n4nnc5cccnc54)cc3)[C@@H]3CC[C@@H](C)NC3)c12]

Example 226 was synthesized following the procedure for Example 50 by substituting tert-butyl (R)-3-aminopiperidine-1-carboxylate with tert-butyl (2R,5R)-5-amino-2-methylpiperidine-1-carboxylate, provided the title compound hydrochloride salt as a white solid. LC-MS: [M+H⁺]=478.0.

### Synthesis of(S)-4-(3H-[1,2,3]triazolo[4,5-b]pyridin-3-yl)-N-(azepan-3-yl)-N-(8-methylisoquinolin-1-yl)benzamide (Example 227)

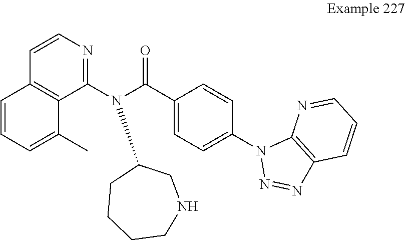 [SMILES: Cc1cccc2ccnc(N(C(=O)c3ccc(-n4nnc5cccnc54)cc3)[C@H]3CCCCNC3)c12]

Example 227 was synthesized following the procedure for Example 50 by substituting tert-butyl (R)-3-aminopiperidine-1-carboxylate with tert-butyl(S)-3-aminoazepane-1-carboxylate, provided the title compound hydrochloride salt as a white solid. LC-MS: [M+H⁺]=478.3.

### Synthesis of(S)-4-(3H-[1,2,3]triazolo[4,5-b]pyridin-3-yl)-N-(azepan-4-yl)-N-(8-methylisoquinolin-1-yl)benzamide (Example 228)

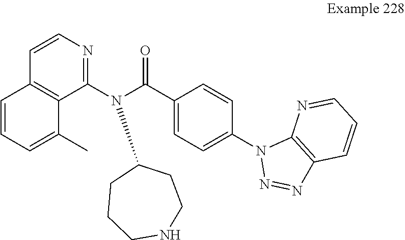 [SMILES: Cc1cccc2ccnc(N(C(=O)c3ccc(-n4nnc5cccnc54)cc3)[C@H]3CCCNCC3)c12]

Example 228 was synthesized following the procedure for Example 50 by substituting tert-butyl (R)-3-aminopiperidine-1-carboxylate with tert-butyl(S)-4-aminoazepane-1-carboxylate, provided the title compound hydrochloride salt as a white solid. LC-MS: [M+H⁺]=478.0.

### Synthesis of (R)-4-(3H-[1,2,3]triazolo[4,5-b]pyridin-3-yl)-N-(azepan-4-yl)-N-(8-methylisoquinolin-1-yl)benzamide (Example 229)

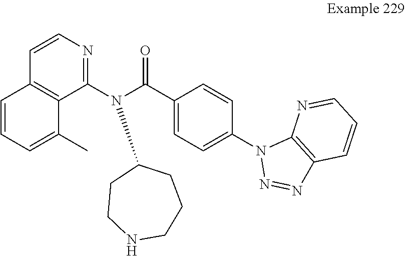 [SMILES: Cc1cccc2ccnc(N(C(=O)c3ccc(-n4nnc5cccnc54)cc3)[C@@H]3CCCNCC3)c12]

Example 229 was synthesized following the procedure for Example 50 by substituting tert-butyl (R)-3-aminopiperidine-1-carboxylate with tert-butyl (R)-4-aminoazepane-1-carboxylate, provided the title compound hydrochloride salt as a white solid. LC-MS: [M+H⁺]=478.3.

### Synthesis of (R)-4-(3H-[1,2,3]triazolo[4,5-b]pyridin-3-yl)-N-(azepan-3-yl)-N-(8-methylisoquinolin-1-yl)benzamide (Example 230)

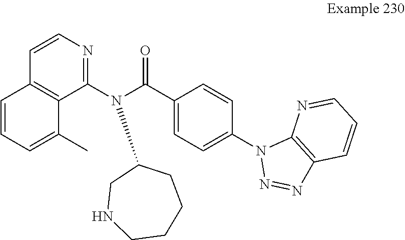 [SMILES: Cc1cccc2ccnc(N(C(=O)c3ccc(-n4nnc5cccnc54)cc3)[C@@H]3CCCCNC3)c12]

Example 230 was synthesized following the procedure for Example 50 by substituting tert-butyl (R)-3-aminopiperidine-1-carboxylate with tert-butyl (R)-3-aminoazepane-1-carboxylate, provided the title compound hydrochloride salt as a white solid. LC-MS: [M+H⁺]=478.3.

### Synthesis of(S)-4-(3H-[1,2,3]triazolo[4,5-b]pyridin-3-yl)-N-(8-methylisoquinolin-1-yl)-N-(pyrrolidin-3-yl)benzamide (Example 231)

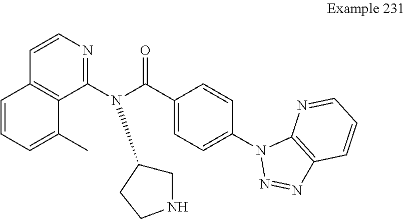 [SMILES: Cc1cccc2ccnc(N(C(=O)c3ccc(-n4nnc5cccnc54)cc3)[C@H]3CCNC3)c12]

Example 231 was synthesized following the procedure for Example 50 by substituting tert-butyl (R)-3-aminopiperidine-1-carboxylate with tert-butyl(S)-3-aminopyrrolidine-1-carboxylate, provided the title compound hydrochloride salt as a white solid. LC-MS: [M+H⁺]=450.3.

### Synthesis of (R)-4-(3H-[1,2,3]triazolo[4,5-b]pyridin-3-yl)-N-(8-methylisoquinolin-1-yl)-N-(pyrrolidin-3-yl)benzamide (Example 232)

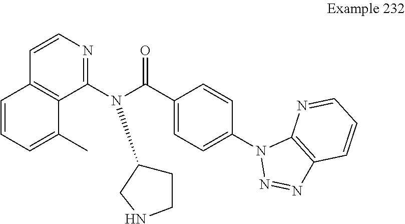 [SMILES: Cc1cccc2ccnc(N(C(=O)c3ccc(-n4nnc5cccnc54)cc3)[C@@H]3CCNC3)c12]

Example 232 was synthesized following the procedure for Example 50 by substituting tert-butyl (R)-3-aminopiperidine-1-carboxylate with tert-butyl (R)-3-aminopyrrolidine-1-carboxylate, provided the title compound hydrochloride salt as a white solid. LC-MS: [M+H⁺]=450.3.

### Synthesis of (R)—N-(8-methylisoquinolin-1-yl)-N-(piperidin-3-yl)-4-(pyridin-3-yl)benzamide (Example 233)

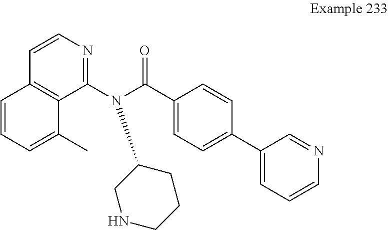 [SMILES: Cc1cccc2ccnc(N(C(=O)c3ccc(-c4cccnc4)cc3)[C@@H]3CCCNC3)c12]

Example 233 was synthesized following the procedure for Example 238 by substituting 4-(pyrazin-2-yl)benzoic acid with 4-(pyridin-3-yl)benzoic acid, provided the title compound hydrochloride salt as a white solid. LC-MS: [M+H⁺]=423.2.

### Synthesis of (R)—N-(8-methylisoquinolin-1-yl)-N-(piperidin-3-yl)-4-(2H-tetrazol-2-yl)benzamide (Example 234)

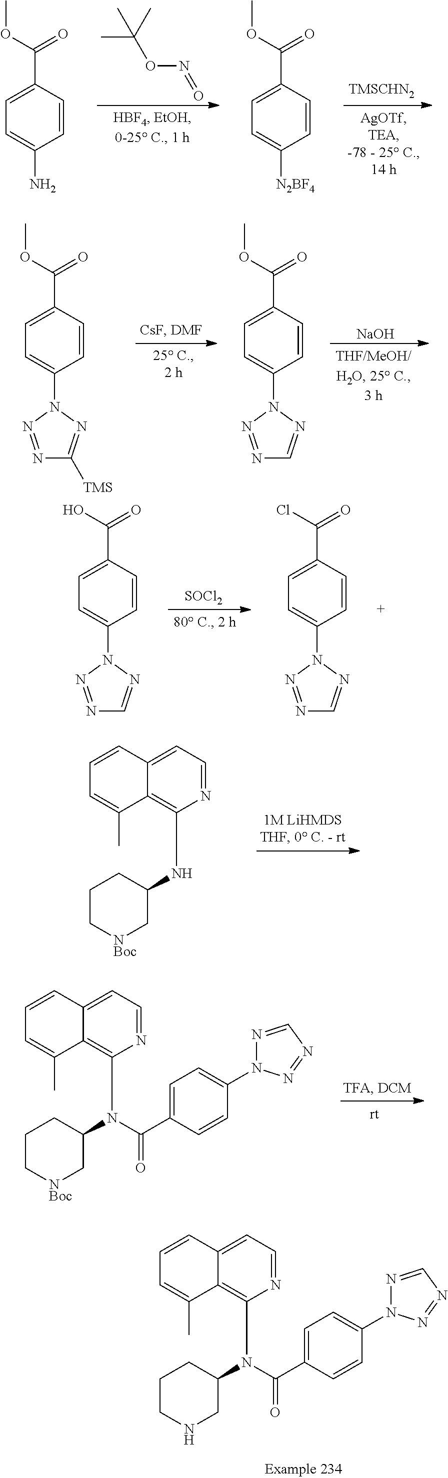

Step 1: To a mixture of methyl 4-aminobenzoate (2.2 g, 14.55 mmol) and trifluoroborane-hydrofluoride (3.78 mL, 29.11 mmol, 48% purity) in EtOH (5 mL) at 0° C. was added tert-butyl nitrite (3.46 mL, 29.11 mmol) dropwise under N₂ atmosphere. The mixture was stirred at 25° C. for 1 h and followed by dropwise addition of MTBE (50 mL). The resulting reaction was stirred for 0.5 h. The precipitate was collected by filtration and dried to give crude methyl (E)-4-((tetrafluoro-λ⁵-boraneyl)diazenyl)benzoate (2.55 g) as a white solid. ¹H NMR: (400 MHz, D₂O) δ 8.61-8.36 (m, 4H), 3.91 (s, 3H).

### Step 2:

To a mixture of methyl (E)-4-((tetrafluoro-λ⁵-boraneyl)diazenyl)benzoate (2 g, 8.0 mmol) and (2,2,2-trifluoroacetyl)oxysilver (2.12 g, 9.60 mmol) in THF (60 mL) at −78° C. under N₂ atmosphere was dropwise added TEA (1.67 mL, 12.0 mmol). The mixture was stirred at −78° C. for 12 min and followed by dropwise addition of TMSCHN₂ (2 M, 4.40 mL). The mixture was stirred at −78° C. for 1 h and warmed to 25° C. and stirred for 13 h. The mixture was poured into ice-water (20 mL) while stirring for 10 min. The aqueous phase was extracted with ethyl acetate (2×20 mL). The combined organic layer was washed with brine (2×20 mL), dried with anhydrous Na₂SO₄, filtered, and concentrated. The residue was purified by flash silica gel chromatography (0-15% ethyl acetate in petroleum ether) to provide methyl 4-(5-(trimethylsilyl)-2H-tetrazol-2-yl)benzoate (770 mg) as a yellow solid. LC-MS: [M+H⁺]=277.0. ¹H NMR (400 MHZ, CDCl₃) δ 8.33-8.23 (m, 4H), 3.99 (s, 3H), 0.53-0.44 (m, 9H).

### Step 3:

To a mixture of methyl 4-(5-(trimethylsilyl)-2H-tetrazol-2-yl)benzoate (960 mg, 3.47 mmol) in DMF (15 mL) at 25° C. under N₂ atmosphere was added CsF (10.42 mmol) in one portion. The mixture was stirred at 25° C. for 2 h, then poured into ice-water (30 mL) and stirred for 10 min. The aqueous phase was extracted with ethyl acetate (2×20 mL). The combined organic layer was washed with brine (2×10 mL), dried with anhydrous Na₂SO₄, filtered, and concentrated. The residue was purified by flash silica gel chromatography (0-15% ethyl acetate in petroleum ether) to provide methyl 4-(2H-tetrazol-2-yl)benzoate (480 mg) as a yellow solid. LC-MS: [M+H⁺]=205.1.

### Step 4:

To a mixture of methyl 4-(2H-tetrazol-2-yl)benzoate (480 mg, 2.35 mmol) in MeOH (5 mL), H₂O (5 mL) and THF (5 mL) 25° C. was added NaOH (470.13 mg, 11.75 mmol. The mixture was stirred at 25° C. for 3 h and then concentrated under reduced pressure. The residue was re-dissolved in water (2 mL) and acidified to pH 3 with 1N aq. HCl solution. The precipitate was collected by filtration and dried to afford 4-(2H-tetrazol-2-yl)benzoic acid (420 mg) as a yellow solid. ¹H NMR (400 MHz, DMSO-d₆) δ 13.42 (br s, 1H), 9.34 (s, 1H), 8.27-8.20 (m, 4H).

### Steps 5-7:

Example 234 was synthesized following the procedure for Example 218 by substituting 4-(3H-[1,2,3]triazolo[4,5-b]pyridin-3-yl)-2,6-difluorobenzoic acid with 4-(2H-tetrazol-2-yl)benzoic acid, provided the title compound hydrochloride salt as a white solid. LC-MS: [M+H⁺]=414.3.

### Synthesis of (R)—N-(8-methylisoquinolin-1-yl)-N-(piperidin-3-yl)-4-(2H-1,2,3-triazol-2-yl)benzamide (Example 235)

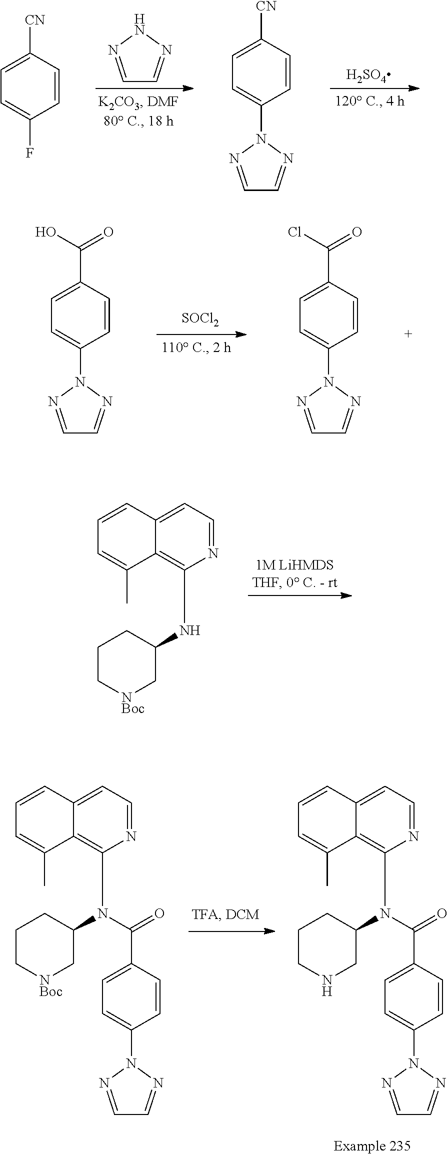

### Step 1:

To a solution of 4-fluorobenzonitrile (4 g, 33.03 mmol) and 2H-triazole (2.60 g, 37.65 mmol) in DMF (10 mL) at room temperature was added K₂CO₃ (9.13 g, 66.05 mmol). The mixture was heated to 80° C. and stirred for 18 h under N₂ atmosphere. The reaction mixture was diluted with water (40 mL) and extracted with EtOAc (3×50 mL). The combined organic layer was dried over Na₂SO₄, filtered, and concentrated. The residue was purified by flash silica gel chromatography (0-30% Ethyl acetate in Petroleum ether) to give 4-(2H-1,2,3-triazol-2-yl)benzonitrile (1.8 g) as a white solid. ¹H NMR (400 MHZ, CDCl₃) δ 8.24 (d, J=8.8 Hz, 2H), 7.89 (s, 2H), 7.80 (d, J=8.7 Hz, 2H).

### Step 2:

A solution of 4-(2H-1,2,3-triazol-2-yl)benzonitrile (1.8 g, 10.58 mmol) in H₂SO₄ (30 mL, 50% content) was stirred at 120° C. for 4 h. The reaction mixture was cooled to room temperature before it was poured into a mixture of ice and water (200 mL). The precipitate was collected by filtration, and dried to give 4-(2H-1,2,3-triazol-2-yl)benzoic acid (1.8 g, 8.28 mmol) as a white solid. ¹H NMR (400 MHZ, CD₃OD) δ ppm 8.21 (overlapping s, 4H), 7.99 (s, 2H).

### Steps 3-5:

Example 235 was prepared following the procedure for Example 218 by substituting 4-(3H-[1,2,3]triazolo[4,5-b]pyridin-3-yl)-2,6-difluorobenzoic acid with 4-(2H-1,2,3-triazol-2-yl)benzoic acid, provided the title compound hydrochloride salt as a white solid. LC-MS: [M+H⁺]=413.1.

### Synthesis of (R)-4-(4-methyl-1H-1,2,3-triazol-1-yl)-N-(8-methylisoquinolin-1-yl)-N-(piperidin-3-yl)benzamide (Example 236)

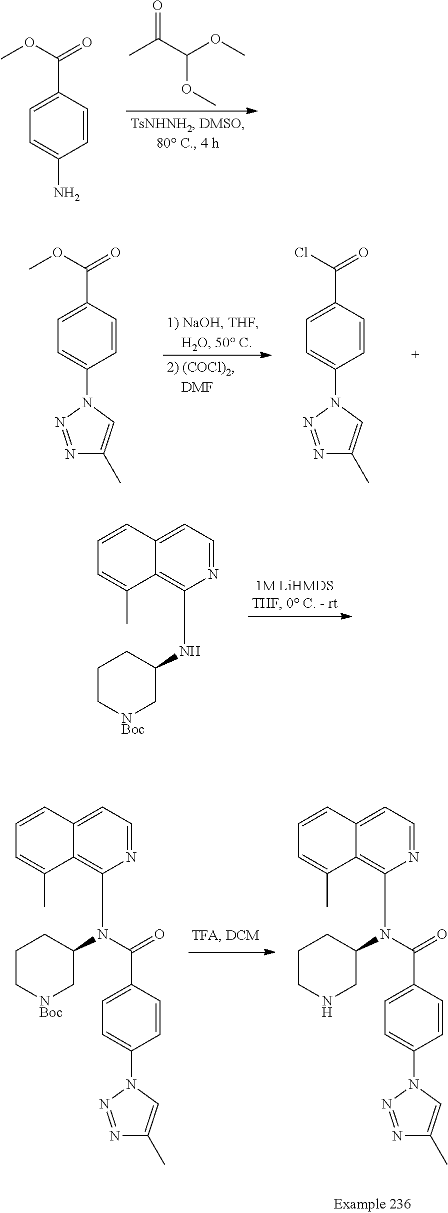

### Step 1:

TsNHNH₂ (5.60 g, 30.07 mmol) was added to a solution of 1,1-dimethoxypropan-2-one (3.55 g, 30.07 mmol) in DMSO (50 mL). The mixture was stirred at 20° C. for 1 h and followed by addition of methyl 4-aminobenzoate (5 g, 33.08 mmol). The mixture was heated at 80° C. for 4 h and then cooled to room temperature. The precipitate was collected by filtration, washed with EtOAc (10 mL), and dried to give methyl 4-(4-methyl-1H-1,2,3-triazol-1-yl)benzoate (3.4 g) as a white solid. LC-MS: [M+H⁺]=218.0.

### Step 2:

A mixture of methyl 4-(4-methyl-1H-1,2,3-triazol-1-yl)benzoate (3 g, 13.81 mmol) in THF (120 mL) and aq. NaOH (1 M, 27.62 mL) was stirred at 50° C. for 0.5 h. The mixture was concentrated, diluted with water and then adjusted to pH 2 with 1M aq. HCl solution. The precipitate was collected and dried to give 4-(4-methyl-1H-1,2,3-triazol-1-yl)benzoic acid (2.73 g) as a white solid. LC-MS: [M+H⁺]=204.0.

### Steps 3-5:

Example 236 was synthesized following the procedure for Example 218 by substituting 4-(3H-[1,2,3]triazolo[4,5-b]pyridin-3-yl)-2,6-difluorobenzoic acid with 4-(4-methyl-1H-1,2,3-triazol-1-yl)benzoic acid, provided the title compound hydrochloride salt as a white solid. LC-MS: [M+H⁺]=427.1.

### Synthesis of (R)—N-(8-methylisoquinolin-1-yl)-N-(piperidin-3-yl)-4-(pyridazin-3-yl)benzamide (Example 237)

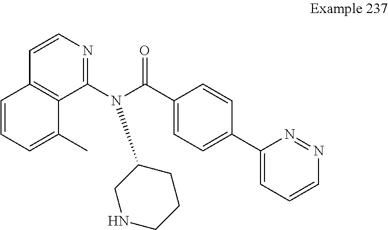 [SMILES: Cc1cccc2ccnc(N(C(=O)c3ccc(-c4cccnn4)cc3)[C@@H]3CCCNC3)c12]

Example 237 was synthesized following the procedure for Example 238 by substituting 2-chloropyrazine with 3-chloropyridazine, to provide the title compound as a hydrochloride salt and as a white solid. LC-MS: [M+H⁺]=424.2.

### Synthesis of (R)—N-(8-methylisoquinolin-1-yl)-N-(piperidin-3-yl)-4-(pyrazin-2-yl)benzamide (Example 238)

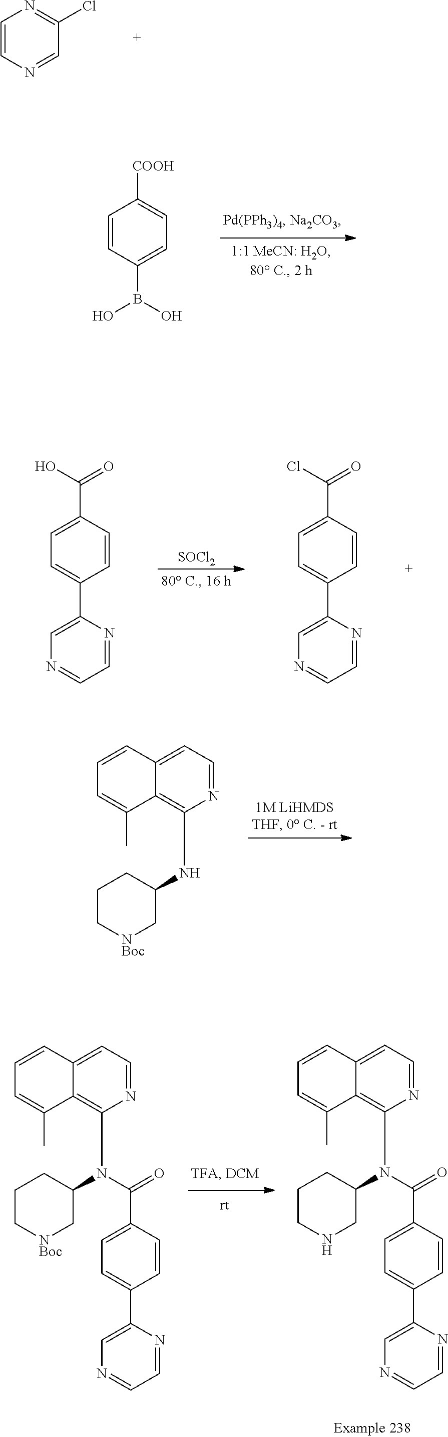

### Step 1:

A mixture of 2-chloropyrazine (580 mg, 5.06 mmol), 4-boronobenzoic acid (840.32 mg, 5.06 mmol), Na₂CO₃ (1.61 g, 15.19 mmol) and Pd(PPh₃) 4 (292.59 mg, 253.20 μmol) in MeCN (7 mL) and H₂O (7 mL) was purged with nitrogen for 10 min and then stirred at 80° C. for 2 h under N₂ atmosphere. The reaction was cooled to 25° C., diluted with H₂O (20 mL), and extracted with DCM (3×80 mL). The aq. phase was acidified to pH 2 with 1.5M aq. HCl. The precipitate was collected by filtration and dried to give crude 4-(pyrazin-2-yl)benzoic acid (763 mg) as an off-white solid. LC-MS: [M+H⁺]=200.9. ¹H NMR (400 MHz, DMSO-d₆) δ 9.33 (d, J=1.3 Hz, 1H), 8.87-8.61 (m, 2H), 8.26 (d, J=8.3 Hz, 2H), 8.08 (d, J=8.4 Hz, 2H).

### Steps 2-4:

Example 238 was synthesized following the procedure for Example 218 by substituting 4-(3H-[1,2,3]triazolo[4,5-b]pyridin-3-yl)-2,6-difluorobenzoic acid with 4-(pyrazin-2-yl)benzoic acid, provided the title compound hydrochloride salt as a white solid. LC-MS: [M+H⁺]=424.1.

### Synthesis of (R)-4-(1-methyl-1H-pyrazol-4-yl)-N-(8-methylisoquinolin-1-yl)-N-(piperidin-3-yl)benzamide (Example 239)

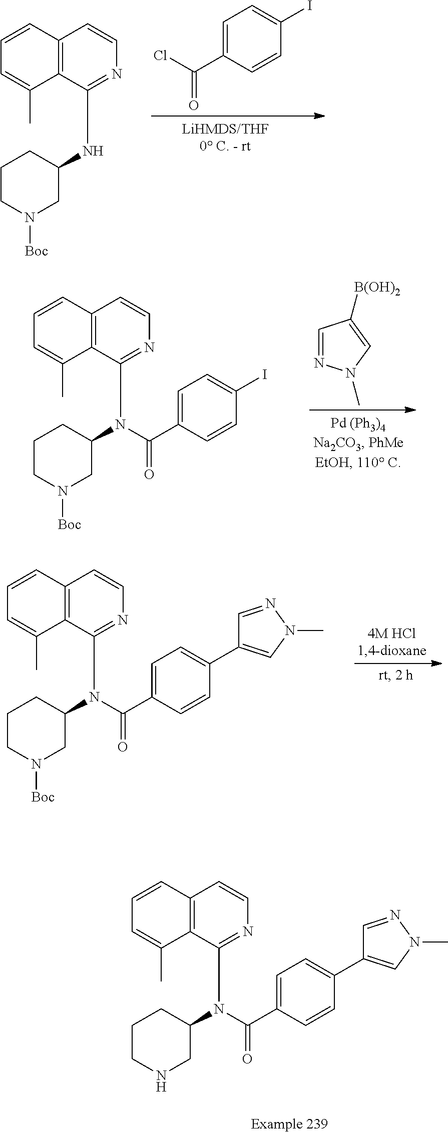

Example 239 was synthesized following the procedure for Example 216 by substituting tert-butyl (R)-3-(2-fluoro-4-iodo-N-(8-methylisoquinolin-1-yl)benzamido)piperidine-1-carboxylate and (1-methyl-5-phenyl-1H-pyrazol-4-yl)boronic acid with tert-butyl (R)-3-(4-iodo-N-(8-methylisoquinolin-1-yl)benzamido)piperidine-1-carboxylate and (1-methyl-1H-pyrazol-4-yl)boronic acid, provided the title compound formic acid salt as a white solid. LC-MS: [M+H⁺]=426.2. ¹H NMR (DMSO-d₆, 400 MHZ,) 8.53 (d, J=5.2 Hz, 1H), 8.28 (s, 1H), 8.00 (s, 1H), 7.83 (d, J=5.6 Hz, 1H), 7.74-7.71 (m, 2H), 7.51-7.47 (m, 1H), 7.40-7.36 (m, 1H), 7.17 (d, J=8.4 Hz, 2H), 6.97-6.93 (m, 2H), 4.71-4.53 (m, 1H), 3.76 (s, 3H), 3.60-3.53 (m, 1H), 3.28-3.23 (m, 1H), 2.99-2.89 (m, 1H), 2.66 (s, 3H), 2.24-2.19 (m, 1H), 178-1.51 (m, 3H), 1.13-1.03 (m, 1H).

### Synthesis of (R)—N-(8-methylisoquinolin-1-yl)-N-(piperidin-3-yl)-3,4-dihydroisoquinoline-2 (1H)-carboxamide (Example 240)

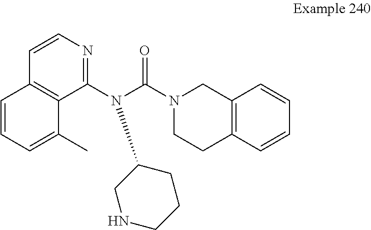 [SMILES: Cc1cccc2ccnc(N(C(=O)N3CCc4ccccc4C3)[C@@H]3CCCNC3)c12]

Example 240 was prepared according to the procedure described for Example 199 by substituting 2-methyl-4-(piperidin-4-yl)pyridine with 1,2,3,4-tetrahydroisoquinoline, provided the title compound hydrochloride salt as a white solid. LC-MS: [M+H⁺]=401.4.

### Synthesis of (R)-4-(3H-[1,2,3]triazolo[4,5-b]pyridin-3-yl)-N-(8-methylisoquinolin-1-yl)-N-(piperidin-3-yl)benzamide (Example 241)

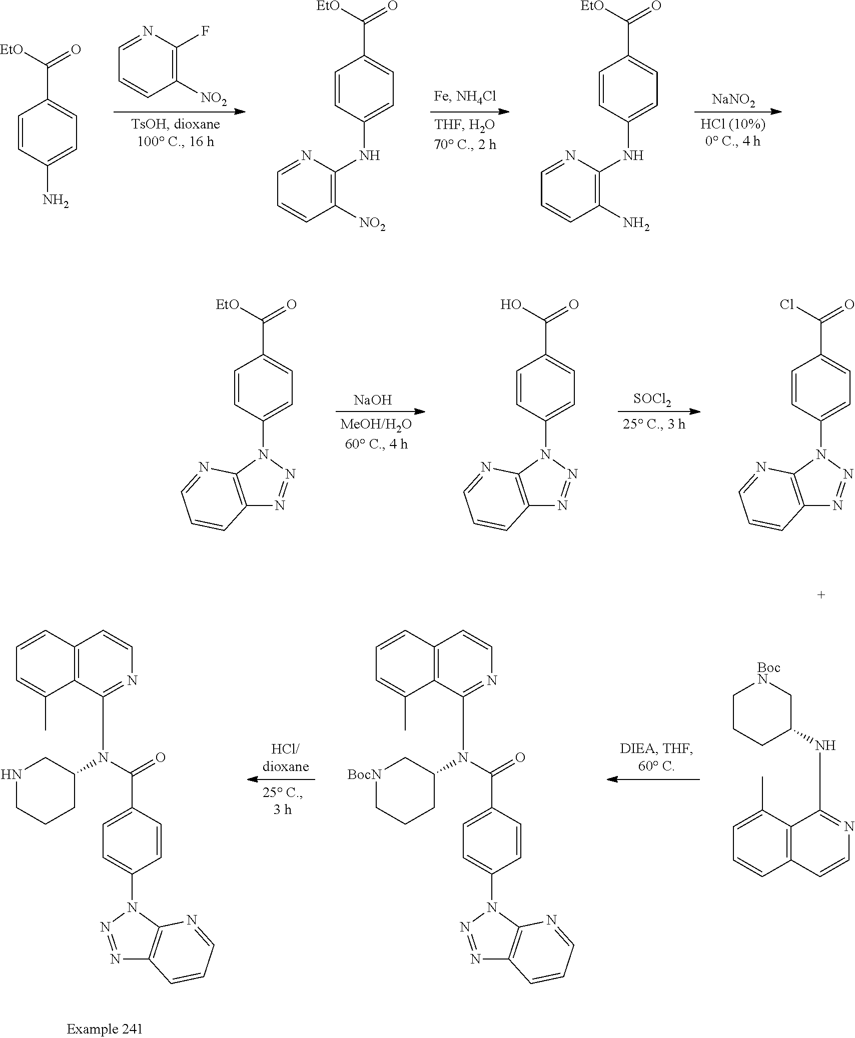

### Step 1:

To a solution of 1-chloro-8-methylisoquinoline (10 g, 56.3 mmol) and tert-butyl (R)-3-aminopiperidine-1-carboxylate (16.9 g, 84.4 mmol) in THF (200 mL) was added t-BuONa (16.2 g, 168 mmol) and BrettPhos Pd G3 (1.53 g, 1.69 mmol). The mixture was stirred at 80° C. for 16 h. The reaction mixture was filtered, and the filtrate was concentrated. The residue was purified by column chromatography (SiO₂, Petroleum ether/EtOAc=20/1 to 5/1) to afford tert-butyl (R)-3-((8-methylisoquinolin-1-yl)amino)piperidine-1-carboxylate (18 g, 93.6% yield) as a brown oil. LC-MS: [M+H⁺]=342.2.

### Step 2:

To a solution of ethyl 4-aminobenzoate (48.4 g, 293 mmol, 61.2 mL) in dioxane (1 L) was added TsOH·H₂O (55.7 g, 293 mmol) and 2-fluoro-3-nitropyridine (50 g, 351 mmol). The mixture was stirred at 100° C. for 16 h before it was concentrated. The residue was diluted with H₂O (1 L) and extracted with EtOAc (3×1 L). The combined organic layer was washed with brine (500 mL), dried over MgSO₄, filtered and concentrated. The crude product was purified by flash silica gel chromatography (ISCO®; 330 g SepaFlash® Silica Flash Column, Eluent of 0˜10% DCM: MeOH) to provide ethyl 4-((3-nitropyridin-2-yl)amino)benzoate (40 g, 47%) as a yellow solid. LC-MS: [M+H⁺]=287.9. ¹H NMR (400 MHz, DMSO-d₆): δ ppm 10.12 (s, 1H), 8.64-8.54 (m, 2H), 7.99-7.91 (m, 2H), 7.91-7.85 (m, 2H), 7.12 (dd, J=4.7, 8.2 Hz, 1H), 4.31 (q, J=7.1 Hz, 2H), 1.33 (t, J=7.1 Hz, 3H).

### Step 3:

To a solution of Ethyl 4-((3-nitropyridin-2-yl)amino)benzoate (40 g, 139 mmol) in THF (250 mL) and H₂O (250 mL) was added Fe (77.7 g, 1.39 mol) and NH₄Cl (74.4 g, 1.39 mol, 10 eq). The mixture was stirred at 70° C. for 3 h before it was concentrated. The residue was diluted with H₂O (500 mL) and extracted with EtOAc (500 mL). The organic layer was washed with brine (250 mL), dried over MgSO₄, filtered and concentrated to give ethyl 4-((3-aminopyridin-2-yl)amino)benzoate (35 g, 97.7% yield) as a brown solid. LC-MS: [M+H⁺]=258.0. ¹H NMR (400 MHZ, DMSO-d₆): δ ppm 8.24 (s, 1H), 7.83 (d, J=8.8 Hz, 2H), 7.70 (d, J=8.8 Hz, 2H), 7.58 (dd, J=1.6, 4.7 Hz, 1H), 6.98 (dd, J=1.6, 7.7 Hz, 1H), 6.74 (dd, J=4.8, 7.7 Hz, 1H), 5.18 (s, 2H), 4.26 (q, J=7.1 Hz, 2H), 4.11 (q, J=5.3 Hz, 1H), 3.18 (d, J=5.3 Hz, 2H), 1.31 (t, J=7.1 Hz, 3H).

### Step 4:

To a solution of Ethyl 4-((3-aminopyridin-2-yl)amino)benzoate (35 g, 136 mmol) in 10% aq. HCl (400 mL) was added NaNO₂ (14 g, 204 mmol). The mixture was stirred at 0° C. for 4 hrs. The reaction mixture was filtered and concentrated to give crude ethyl 4-(3H-[1,2,3]triazolo[4,5-b]pyridin-3-yl)benzoate (35 g, 96%) as a brown solid.

### Step 5:

To a solution of crude ethyl 4-(3H-[1,2,3]triazolo[4,5-b]pyridin-3-yl)benzoate (35 g, 130 mmol) in MeOH (350 mL) and H₂O (100 mL) was added NaOH (15.6 g, 391 mmol). The mixture was stirred at 60° C. for 6 h before it was concentrated. The residue was diluted with water (100 mL) acidified to pH 6 with dilute aq. HCl. The precipitate was collected and dried to provide crude 4-(3H-[1,2,3]triazolo[4,5-b]pyridin-3-yl)benzoic acid (25 g, 79.7%) as a solid, which was used in the next step without further purification.

### Step 6:

To a solution of 4-(3H-[1,2,3]triazolo[4,5-b]pyridin-3-yl)benzoic acid (6 g, 24.9 mmol) in DCM (30 mL) was added SOCl₂ (29.7 g, 249 mmol). The mixture was stirred at 20° C. for 2 h. The reaction mixture was concentrated to provide crude 4-(3H-[1,2,3]triazolo[4,5-b]pyridin-3-yl)benzoyl chloride (6 g, 92%) as a solid.

### Step 7:

To a solution of tert-butyl (R)-3-((8-methylisoquinolin-1-yl)amino)piperidine-1-carboxylate (2.54 g, 7.45 mmol) in THF (10 mL) was added DIEA (4.92 g, 38 mmol) and crude 4-(3H-[1,2,3]triazolo[4,5-b]pyridin-3-yl)benzoyl chloride (2.31 g, 8.94 mmol). The mixture was stirred at 60° C. for 10 h before it was concentrated. The residue was diluted with H₂O (20 mL) and extracted with EtOAc (3×20 mL). The combined organic layer was washed with brine (10 mL), dried over MgSO₄, filtered and concentrated. The residue was purified by flash silica gel chromatography (Eluent of 40% EtOAc/Petroleum ether) to provide tert-butyl (R)-3-(4-(3H-[1,2,3]triazolo[4,5-b]pyridin-3-yl)-N-(8-methylisoquinolin-1-yl)benzamido)piperidine-1-carboxylate (3 g, 69.9%) as a yellow solid. LC-MS: [M+H⁺]=564.2. ¹H NMR (400 MHz, DMSO-d₆): δ ppm 8.79 (br d, J=2.8 Hz, 1H), 8.65 (br d, J=8.3 Hz, 1H), 8.56-8.51 (m, 1H), 7.97 (br d, J=7.9 Hz, 2H), 7.87 (br d, J=4.4 Hz, 1H), 7.80-7.72 (m, 1H), 7.62-7.47 (m, 3H), 7.42 (br d, J=7.0 Hz, 1H), 7.32 (br d, 7.3 Hz, 2H), 5.76 (s, 1H), 4.68-4.50 (m, 2H), 4.06-3.99 (m, 1H), 3.97-3.87 (m, 2H), 2.81 (br s, 1H), 2.73 (br s, 2H), 1.57 (br s, 2H), 1.46 (br s, 5H), 1.37-1.30 (m, 3H), 1.21-1.09 (m, 2H).

### Step 8:

The title compound hydrochloride salt was obtained as an off-white solid by deprotection Method A used in Example 97. LC-MS: [M+H⁺]=464.2. ¹H NMR (400 MHZ, DMSO-d₆): δ ppm 9.43-9.21 (m, 1H), 9.17-8.92 (m, 1H), 8.82-8.78 (m, 1H), 8.66 (dd, J=1.3, 8.3 Hz, 1H), 8.59-8.52 (m, 1H), 8.00-7.96 (m, 2H), 7.94-7.89 (m, 1H), 7.78 (d, J=8.0 Hz, 1H), 7.58 (dd, J=4.6, 8.3 Hz, 1H), 7.53 (t, J=7.4 Hz, 1H), 7.50-7.43 (m, 1H), 7.35-7.29 (m, 2H), 5.76 (s, 1H), 5.04-4.83 (m, 1H), 3.84-3.75 (m, 1H), 3.31-3.15 (m, 2H), 2.84-2.78 (m, 2H), 2.77-2.70 (m, 3H), 1.98-1.78 (m, 2H), 1.73-1.63 (m, 1H), 1.30-1.13 (m, 1H).

### Synthesis of (R)-4-(3H-[1,2,3]triazolo[4,5-b]pyridin-3-yl)-2-fluoro-N-(8-methylisoquinolin-1-yl)-N-(piperidin-3-yl)benzamide (Example 242)

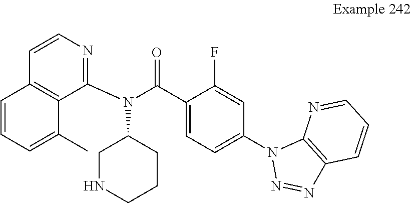 [SMILES: Cc1cccc2ccnc(N(C(=O)c3ccc(-n4nnc5cccnc54)cc3F)[C@@H]3CCCNC3)c12]

Example 242 was prepared following the procedure for Example 241 by substituting ethyl 4-aminobenzoate with methyl 4-amino-2-fluorobenzoate to yield 5.2 g of the title compound as a hydrochloride salt as a white solid. LC-MS: [M+H⁺]=482.2. ¹H NMR (400 MHz, CD₃OD) δ ppm 8.74 (dd, J=1.3, 4.5 Hz, 1H), 8.51-8.44 (m, 2H), 8.03-7.95 (m, 1H), 7.84 (dd, J=1.8, 8.5 Hz, 1H), 7.78-7.72 (m, 1H), 7.68-7.62 (m, 1H), 7.54-7.47 (m, 3H), 7.16-7.07 (m, 1H), 5.25-4.95 (m, 1H), 4.13-3.59 (m, 2H), 3.50-3.41 (m, 1H), 3.10-3.01 (m, 1H), 2.93-2.87 (m, 3H), 2.81-2.67 (m, 1H), 2.26-1.94 (m, 3H), 1.67-1.26 (m, 1H).

### Synthesis of (R)—N-(8-methylisoquinolin-1-yl)-4-(3-methylisoxazol-5-yl)-N-(piperidin-3-yl)piperidine-1-carboxamide (Example 243) (repeat production of Example 61)

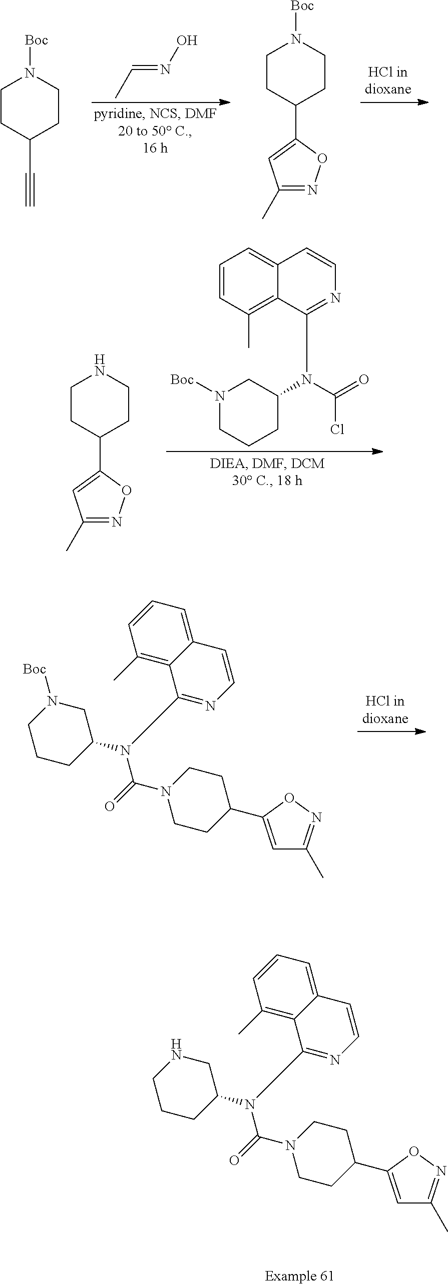

### Step 1:

To a solution of (1E)-acetaldehyde oxime (8.47 g, 143.35 mmol) in DMF (100 mL) was added TEA (29.01 g, 286.69 mmol) and NCS (21.06 g, 157.68 mmol). The mixture was stirred at 20° C. for 1 h and followed by addition of tert-butyl 4-ethynylpiperidine-1-carboxylate (30 g, 143.35 mmol). The reaction mixture was stirred at 20° C. for 1 h and followed by addition of pyridine (1.13 g, 14.33 mmol). The reaction was stirred at 50° C. for 14 h before it was cooled to room temperature and concentrated. The residue was purified by flash column chromatography (SiO₂, Petroleum ether/EtOAc=20/1 to 5/1) to afford tert-butyl 4-(3-methylisoxazol-5-yl)piperidine-1-carboxylate (12 g, 31%) as a solid.

### Step 2:

A solution of tert-butyl 4-(3-methylisoxazol-5-yl)piperidine-1-carboxylate (22 g, 82.60 mmol) in 4M HCl in dioxane (500 mL) was stirred at 20° C. for 0.5 h. The reaction mixture was concentrated under reduced pressure to afford crude 3-methyl-5-(4-piperidyl) isoxazole hydrochloride salt (12 g, 87%) as a white solid.

### Step 3:

To a solution of tert-butyl (3R)-3-[chlorocarbonyl-(8-methyl-1-isoquinolyl)amino]piperidine-1-carboxylate (7.8 g, 19.31 mmol) in DCM (80 mL) and DMF (8 mL) at room temperature was added 3-methyl-5-(4-piperidyl) isoxazole hydrochloride salt (7.83 g, 38.62 mmol) and DIEA (12.48 g, 96.56 mmol). The mixture was stirred at 30° C. for 12 h before it was concentrated under reduced pressure. The residue was purified by flash column chromatography (SiO₂, petroleum ether/ethyl acetate=20/1 to 3/1) to afford tert-butyl (3R)-3-[(8-methyl-1-isoquinolyl)-[4-(3-methylisoxazol-5-yl) piperidine-1-carbonyl] amino] piperidine-1-carboxylate (6 g, 58%) as a yellow solid.

### Step 4:

The title compound hydrochloride salt was obtained as a white solid by deprotection Method A used in Example 97. LC-MS: [M+H⁺]=434.3. ¹H NMR (DMSO-d₆, 400 MHZ, 90° C.) δ ppm 9.15 (bs, 1H), 8.86 (bs, 1H), 8.32 (d, J=11.2 Hz, 1H), 7.83 (d, J=8.0 Hz, 1H), 7.74 (d, J=5.2 Hz, 1H), 7.62 (t, J=7.6 Hz, 1H), 7.52-7.49 (m, 1H), 5.81-5.77 (m, 1H), 4.72-4.62 (m, 1H), 3.52-3.34 (m, 4H), 3.24-3.18 (m, 1H), 2.74-2.44 (m, 6H), 2.11 (s, 3H), 1.75-1.25 (m, 4H), 1.55-1.53 (m, 1H), 1.34-1.25 (m, 2H), 0.87-0.85 (m, 1H), 0.46-0.31 (m, 1H).

### Synthesis of (R)-5-(3H-[1,2,3]triazolo[4,5-b]pyridin-3-yl)-N-(8-methylisoquinolin-1-yl)-N-(piperidin-3-yl)picolinamide (Example 244)

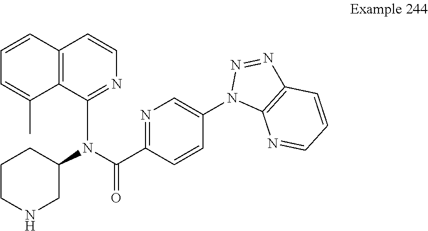 [SMILES: Cc1cccc2ccnc(N(C(=O)c3ccc(-n4nnc5cccnc54)cn3)[C@@H]3CCCNC3)c12]

Example 244 was prepared following the procedure for Example 241 by substituting ethyl 4-aminobenzoate with methyl 5-aminopicolinate to provide the title compound as a hydrochloride salt. LC-MS: [M+H⁺]=465.7. ¹H NMR (CD₃OD, 400 MHz) δ ppm 8.66-8.65 (m, 1H), 8.41-8.39 (m, 1H), 8.34 (d, J=5.6 Hz, 1H), 7.92 (dd, J=11.6 Hz, 2.0 Hz, 1H), 7.81-7.78 (m, 1H), 7.60 (d, J=6.0 Hz, 1H), 7.47-7.41 (m, 2H), 7.27-7.25 (m, 1H), 7.16-7.09 (m, 1H), 7.03-7.01 (m, 1H), 5.05-4.95 (m, 1H), 4.01 (s, 3H), 3.82-3.79 (m, 1H), 3.65-3.60 (m, 1H), 3.25-3.24 (m, 1H), 2.87-2.80 (m, 1H), 1.88-1.76 (m, 3H), 1.33-1.18 (m, 1H).

### Synthesis of (R)-6-(3H-[1,2,3]triazolo[4,5-b]pyridin-3-yl)-N-(8-methylisoquinolin-1-yl)-N-(piperidin-3-yl)nicotinamide (Example 245)

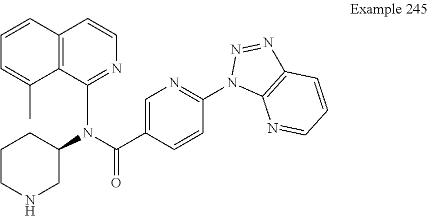 [SMILES: Cc1cccc2ccnc(N(C(=O)c3ccc(-n4nnc5cccnc54)nc3)[C@@H]3CCCNC3)c12]

Example 245 was prepared following the procedure for Example 241 by substituting ethyl 4-aminobenzoate with methyl 6-aminopicolinate to provide the title compound as a hydrochloride salt. LC-MS: [M+H⁺]=465.25. ¹H NMR (CD₃OD, 400 MHz) δ ppm 8.75 (d, J=4 Hz, 1H), 8.55-8.49 (m, 2H), 8.22 (s, 1H), 8.12-8.06 (m, 1H), 7.86-7.81 (m, 1H), 7.79-7.76 (m, 1H), 7.71-7.68 (m, 1H), 7.56-7.53 (m, 1H), 7.50-7.42 (m, 2H), 5.10-5.02 (m, 1H), 4.20-3.98 (m, 1H), 3.46-3.32 (m, 2H), 3.11-3.10 (m, 1H), 2.77 (s, 3H), 1.94-1.81 (m, 3H), 1.37-1.33 (m, 1H).

### Synthesis of (R)-4-(3H-[1,2,3]triazolo[4,5-b]pyridin-3-yl)-N-(8-chloroisoquinolin-1-yl)-2-fluoro-N-(piperidin-3-yl)benzamide (Example 246)

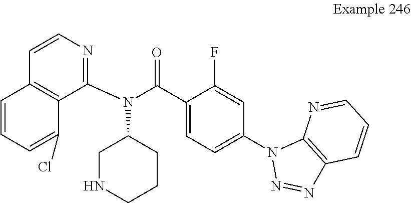 [SMILES: O=C(c1ccc(-n2nnc3cccnc32)cc1F)N(c1nccc2cccc(Cl)c12)[C@@H]1CCCNC1]

Example 246 was prepared following the procedure for Example 241 by substituting 1-chloro-8-methylisoquinoline and 4-aminobenzoate with 1,8-dichloroisoquinoline and 4-amino-2-fluoro-benzoate to provide the title compound as a hydrochloride salt. LC-MS: [M+H⁺]=502.5.

¹H NMR (DMSO-d₆, 400 MHZ) δ ppm 9.52 (d, J=9.2 Hz, 1H), 8.20 (d, J=3.6 Hz, 1H), 8.68 (d, J=8.4 Hz, 1H), 8.61-8.60 (m, 1H), 8.06-7.86 (m, 5H), 7.78-7.59 (m, 2H), 7.39-7.21 (m, 1H), 5.03-4.90 (m, 1H), 3.81-3.78 (m, 1H), 3.57-3.47 (m, 1H), 3.34-3.23 (m, 1H), 2.83-2.67 (m, 1H), 2.08-1.83 (m, 3H), 1.21-1.15 (m, 1H).

### Synthesis of (R)-2-(4-(3H-[1,2,3]triazolo[4,5-b]pyridin-3-yl)-2-fluoro-N-(piperidin-3-yl)benzamido)-N,N-dimethylnicotinamide (Example 247)

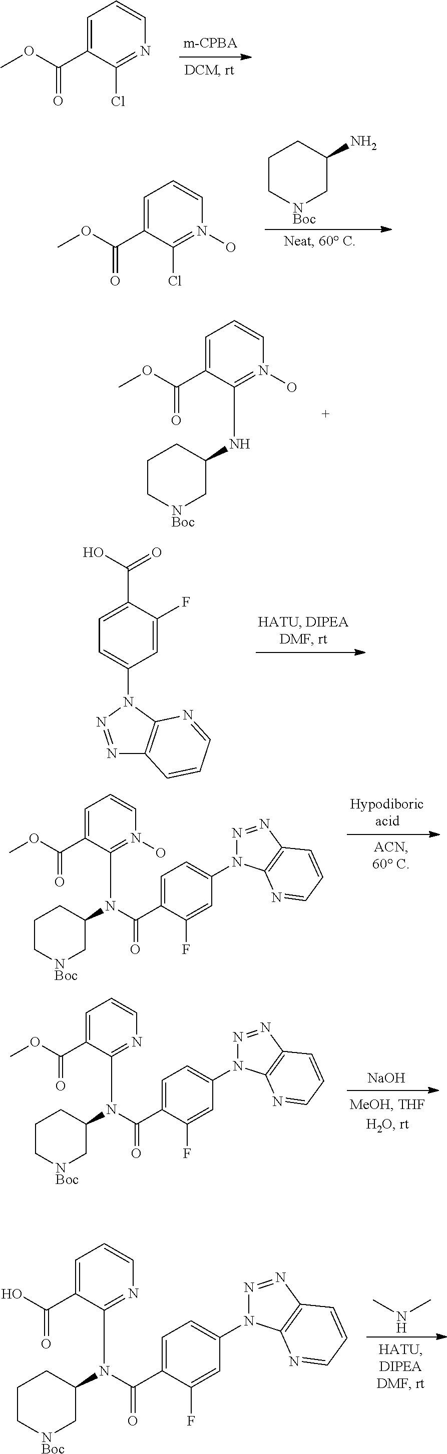

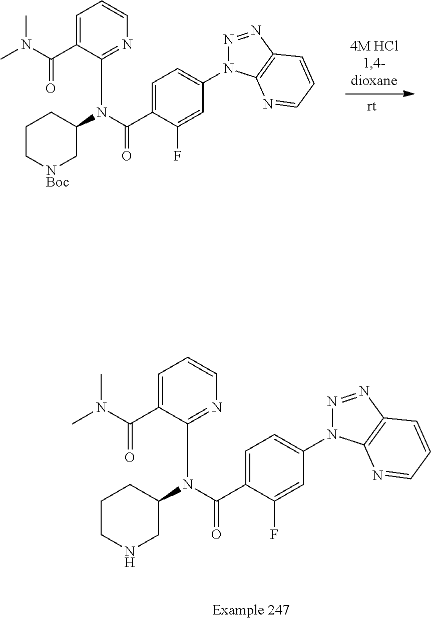 [SMILES: CN(C)C(=O)c1cccnc1N(C(=O)c1ccc(-n2nnc3cccnc32)cc1F)[C@@H]1CCCN(C(=O)OC(C)(C)C)C1 ; CN(C)C(=O)c1cccnc1N(C(=O)c1ccc(-n2nnc3cccnc32)cc1F)[C@@H]1CCCNC1]

### Step 1:

To the stirred solution of methyl 2-chloronicotinate (2.0 g, 11.65 mmol, 1.0 eq) in DCM was added m-CPBA (12.03 g, 69.93 mmol) at 25° C. and stirred for 16 h. The reaction mixture was concentrated and co-distilled with toluene. The residue was purified by Combi-Flash eluting with 100% EtOAc to afford 2-chloro-3-(methoxycarbonyl)pyridine 1-oxide (1.7 g, 78%) as an off-white solid.

### Step 2:

To a mixture of 2-chloro-3-(methoxycarbonyl)pyridine 1-oxide (1.6 g, 8.55 mmol), tert-butyl (R)-3-aminopiperidine-1-carboxylate (3 mL) in a sealed tube was stirred at 60° C. for 4 h. The reaction mixture partitioned between EtOAc (100 mL) and (50 mL) of water. The organic layer was washed with 1N aq. HCl (50 mL), dried over Na₂SO₄, filtered, and concentrated to afford (R)-2-((1-(tert-butoxycarbonyl)piperidin-3-yl)amino)-3-(methoxycarbonyl)pyridinel-oxide (2.9 g) as a brown liquid.

### Step 3:

To a solution of 4-(3H-[1,2,3]triazolo[4,5-b]pyridin-3-yl)-2-fluorobenzoic acid (2.1 g, 8.20 mmol) in DMF (10 mL) were added DIPEA (05 mL, 24.6 mmol) and HATU (9.35 g, 24.6 mmol) at 0° C. and stirred for 15 min and followed by addition of (R)-2-((1-(tertbutoxycarbonyl) piperidin-3-yl)amino)-3-(methoxycarbonyl)pyridine 1-oxide (2.9 g, 8.20 mmol). The reaction mixture was stirred at 25° C. for 4 h before it was quenched with ice cold water. The precipitate was collected by filtration and dried to afford (R)-2-(4-(3H-[1,2,3]triazolo[4,5-b]pyridin-3-yl)-N-(1-(tert-butoxycarbonyl)piperidin-3-yl)-2-fluorobenzamido)-3-(methoxycarbonyl)pyridinel-oxide (4.5 g) as a yellow solid.

### Step 4:

To a solution of (R)-2-(4-(3H-[1,2,3]triazolo[4,5-b]pyridin-3-yl)-N-(1-(tert-butoxycarbonyl)piperidin-3-yl)-2-fluorobenzamido)-3-(methoxycarbonyl)pyridine 1-oxide (4.5 g, 7.58 mmol) in MeCN (50 mL) were added hypodiboric acid (2.02 g, 22.74 mmol) and stirred at 60° C. for 4 h. The reaction mixture was concentrated, diluted with water and with EtOAc (2×100 mL). The combined organic layer was dried over Na₂SO₄, filtered and concentrated. The residue was purified by Combi-Flash eluting with 60-70% EtOAc in hexane first and further purified by prep-HPLC (Conditions: Luna OMEGA C-18 (250×21.2×5 μm), Mobile phase A: 0.1% formic acid in H₂O, Mobile phase B: MeCN (100%), Gradient: T/% B 0/55, 10/85 Flow 15 mL/min. Dilution: MeCN+H₂O+THF). Pure fractions were dried by lyophilization to afford methyl (R)-2-(4-(3H-[1,2,3]triazolo[4,5-b]pyridin-3-yl)-N-(1-(tert-butoxycarbonyl)piperidin-3-yl)-2-fluorobenzamido)nicotinate (350 mg, 60%) as an off-white solid.

### Step 5:

To a solution of methyl (R)-2-(4-(3H-[1,2,3]triazolo[4,5-b]pyridin-3-yl)-N-(1-(tertbutoxycarbonyl)piperidin-3-yl)-2-fluorobenzamido)nicotinate (300 mg, 0.521 mmol) in MeOH (5 mL), THF (10 mL) and H₂O (5 mL) at 20° C. was added NaOH (104 mg, 2.60 mmol). The reaction mixture was stirred at 80° C. for 4 h before it was concentrated. The residue was diluted with water (20 mL) and acidified with 1N aq. HCl to pH˜2 and extracted with EtOAc (100 mL). The organic layer was dried over Na₂SO₄, filtered and concentrated to afford (R)-2-(4-(3H-[1,2,3]triazolo[4,5-b]pyridin-3-yl)-N-(1-(tert-butoxycarbonyl)piperidin-3-yl)-2-fluorobenzamido) nicotinic acid (230 mg, 78%) as an off-white solid. LC-MS: [M+1⁺]=562.3.

### Step 6:

To a solution of (R)-2-(4-(3H-[1,2,3]triazolo[4,5-b]pyridin-3-yl)-N-(1-(tert-butoxycarbonyl)piperidin-3-yl)-2-fluorobenzamido) nicotinic acid (50 mg, 0.089 mmol) in DMF (2 mL) at 0° C. was added DIPEA (0.1 mL, 0.267 mmol) and HATU (101 mg, 0.267 mmol) at 0° C. and stirred for 15 min and followed by addition of dimethylamine hydrochloride salt (10.8 mg, 0.133 mmol). The reaction mixture was stirred at 25° C. for 4 h before it was poured into ice cold water and extracted with EtOAc (100 mL). The organic layer was washed with brine, dried over Na₂SO₄, and concentrated. The residue was purified by prep-HPLC (Conditions: Column: X-SELECT CSH C18 (250*19) mm, 5 μm), Mobile phase A: 0.1% formic acid in H₂O, Mobile phase B: MeCN (100%), Gradient: T/% B: 0/50, 10/75. Flow: 12 mL/min, Dilution: MeCN+H₂O+THF). Pure fractions were dried by lyophilization to afford tert-butyl (R)-3-(4-(3H-[1,2,3]triazolo[4,5-b]pyridin-3-yl)-N-(3-(dimethylcarbamoyl)pyridin-2-yl)-2-fluorobenzamido)piperidine-1-carboxylate (70 mg) as an off-white solid. LC-MS: [M+1+]=489.2.

### Step 7:

To a solution of tert-butyl (R)-3-(4-(3H-[1,2,3]triazolo[4,5-b]pyridin-3-yl)-N-(3-(dimethylcarbamoyl)pyridin-2-yl)-2-fluorobenzamido)piperidine-1-carboxylate (70 mg, 0.118 mmol) in DCM (2 mL) at 0° C. was added 4M HCl in 1,4-dioxane (5 mL). The reaction mixture was stirred at 20° C. for 2 h before it was concentrated. The residue was purified by prep-HPLC (Conditions: Column: X-SELECT CSH C18 (250*19) mm, 5 μm), Mobile phase A: 0.1% formic acid in H₂O, Mobile phase B: MeCN (100%), Gradient: T/% B: 0/25, 2/25, 10/60. Flow: 15 mL/min, Dilution: MeCN+H₂O+THF). Pure fractions were dried by lyophilization to afford (R)-2-(4-(3H-[1,2,3]triazolo[4,5-b]pyridin-3-yl)-2-fluoro-N-(piperidin-3-yl)benzamido)-N,N-dimethylnicotinamide formate salt (25 mg, 50%) as an off-white solid. LC-MS: [M+1⁺]=489.2. ¹H NMR (CD₃OD, 400 MHZ): δ ppm 8.73 (m, 1H), 8.59 (m, 1H), 8.47 (d, J=8.4 Hz, 1H), 8.24-8.20 (m, 1H), 8.06 (m, 1H), 7.71 (m, 1H), 7.51-7.48 (m, 1H), 7.35-7.34 (m, 2H), 4.76-4.67 (m 1H), 3.73-3.52 (m, 1H), 2.96 (s, 6H), 2.82-2.76 (m, 1H), 2.32-2.17 (m, 3H), 1.84-1.73 (m, 2H), 1.50 (m, 1H).

### Synthesis of (R)-4-(3H-[1,2,3]triazolo[4,5-b]pyridin-3-yl)-2-fluoro-N-(3-methyl-3H-imidazo[4,5-c]pyridin-4-yl)-N-(piperidin-3-yl)benzamide (Example 248)

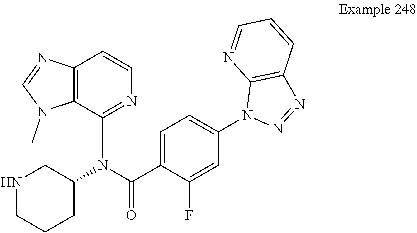 [SMILES: Cn1cnc2ccnc(N(C(=O)c3ccc(-n4nnc5cccnc54)cc3F)[C@@H]3CCCNC3)c21]

Example 248 was prepared according to the procedure described for Example 255 by substituting 7-chloro-1,3-dimethyl-1H-pyrrolo[2,3-c]pyridine with 4-chloro-3-methyl-3H-imidazo-[4,5-c]pyridine, provided the title compound hydrochloride salt as a white solid. LC-MS (ESI⁺): m/z 472.1 [M+H⁺]. ¹⁹F NMR (377 MHz, DMSO-d₆) δ ppm −109.58-−113.20 (m, 1F).

### Synthesis of (R)-4-(3H-[1,2,3]triazolo[4,5-b]pyridin-3-yl)-2-fluoro-N-(1,6-naphthyridin-5-yl)-N-(piperidin-3-yl)benzamide (Example 249)

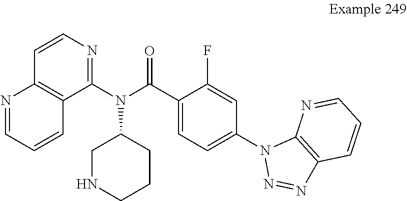 [SMILES: O=C(c1ccc(-n2nnc3cccnc32)cc1F)N(c1nccc2ncccc12)[C@@H]1CCCNC1]

Example 249 was prepared following the procedure for Example 242 by substituting 1-chloro-8-methylisoquinoline with 5-chloro-1,6-naphthyridine to provide the title compound as a hydrochloride salt. LC-MS: [M+H⁺]=469.3. ¹H NMR (CD₃OD, 400 MHZ): δ ppm 9.01 (bs, 1H), 8.79-8.65 (m, 3H), 8.41 (d, J=8.4 Hz, 1H), 7.96-7.83 (m, 3H), 7.76-7.73 (m, 1H), 7.45-7.42 (m, 1H), 7.26-7.25 (m, 1H), 5.22-5.18 (m, 1H), 3.90-3.87 (m, 1H), 3.73-3.61 (m, 1H), 3.28-3.25 (m, 1H), 2.84-2.78 (m, 1H), 1.93-1.84 (m, 3H), 1.24-1.18 (m, 1H).

### Synthesis of (R)-4-(3-(difluoromethyl) isoxazol-5-yl)-N-(8-methylisoquinolin-1-yl)-N-(piperidin-3-yl)piperidine-1-carboxamide ((Example 250)

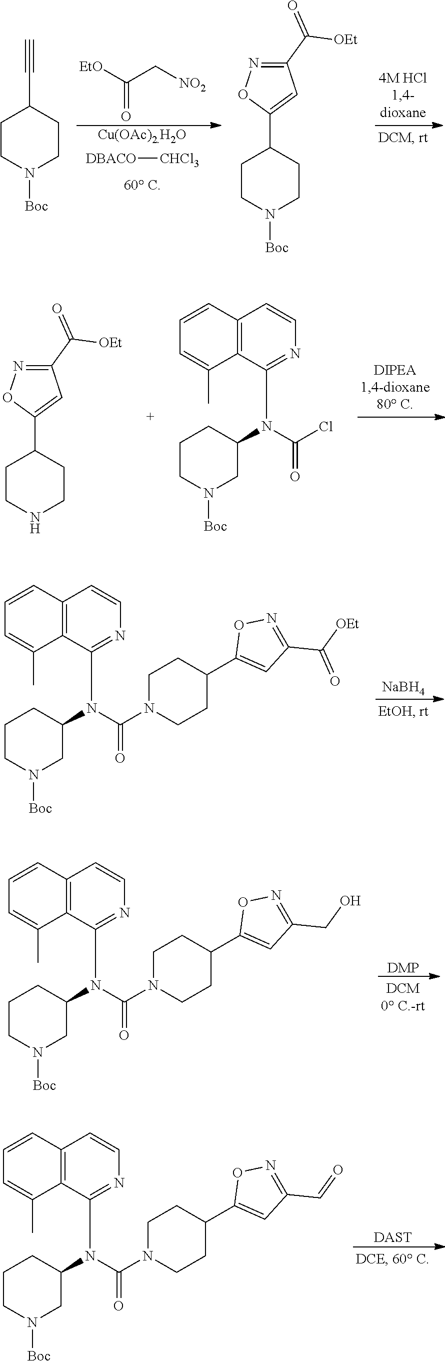

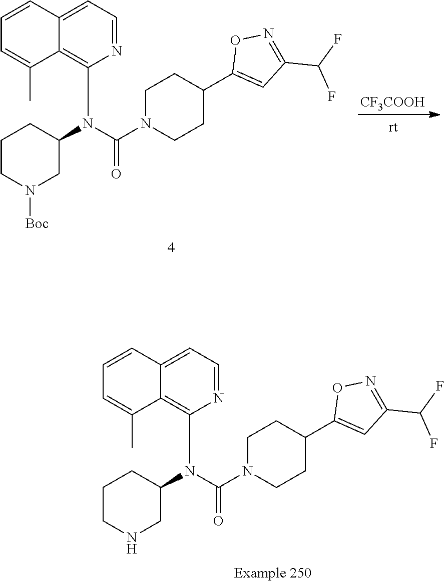 [SMILES: Cc1cccc2ccnc(N(C(=O)N3CCC(c4cc(C(F)F)no4)CC3)[C@@H]3CCCN(C(=O)OC(C)(C)C)C3)c12 ; "CF3COOH" ; Cc1cccc2ccnc(N(C(=O)N3CCC(c4cc(C(F)F)no4)CC3)[C@@H]3CCCNC3)c12]

### Step 1:

To a solution of tert-butyl 4-ethynylpiperidine-1-carboxylate (5 g, 23.9 mmol) in CHCl₃ (30 mL) were added Cu(OAc)₂·H₂O (250 mg, 1.19 mmol), DBACO (1.3 g, 11.9 mmol) and ethyl 2-nitroacetate (9.5 g, 71.7 mmol) at 20° C. The reaction mixture was stirred at 60° C. for 16 h before it was diluted with ice water (100 mL) and extracted with DCM (3×150 mL). The combined organic layer was washed with brine (50 mL), dried over Na₂SO₄, and concentrated. The residue was purified by Combi-Flash eluting with 30% EtOAc in hexane to afford ethyl 5-(1-(tert-butoxycarbonyl)piperidin-4-yl)isoxazole-3-carboxylate (5 g, 64%) as a colorless oil.

### Step 2:

To a solution of ethyl 5-(1-(tert-butoxycarbonyl)piperidin-4-yl)isoxazole-3-carboxylate (2 g, 6.17 mmol) in DCM (10 mL) was added 4M HCl in 1,4-dioxane (10 mL) at 0° C. The reaction was stirred at 25° C. for 2 h before it was concentrated. The residue was washed with diethyl ether and dried to afford ethyl 5-(piperidin-4-yl)isoxazole-3-carboxylate hydrochloride salt (1.4 g, 87%) as an off-white solid. LC-MS: [M+1⁺]=225.2.

### Step 3:

To a solution of ethyl 5-(piperidin-4-yl)isoxazole-3-carboxylate (1.4 g, 6.25 mmol) in 1,4-dioxane (10 mL) at 25° C. was added tert-butyl (R)-3-((chlorocarbonyl)(8-methylisoquinolin-1-yl)amino)piperidine-1-carboxylate (1.8 g, 5.0 mmol) and DIPEA (3.2 mL, 18.7 mmol). The reaction was stirred at 90° C. for 16 h before it was concentrated. The residue was purified by Combi-Flash eluting with 30% EtOAc in hexane to afford ethyl (R)-5-(1-((1-(tert-butoxycarbonyl)piperidin-3-yl)(8-methylisoquinolin-1-yl)carbamoyl)piperidin-4-yl)isoxazole-3-carboxylate (1.2 g, 34%) as an off-white solid. LC-MS: [M+1⁺]=592.3.

### Step 4:

To a solution of ethyl (R)-5-(1-((1-(tert-butoxycarbonyl)piperidin-3-yl)(8-methylisoquinolin-1-yl)carbamoyl)piperidin-4-yl)isoxazole-3-carboxylate (500 mg, 0.84 mmol) in Ethanol (5 mL) was added NaBH₄ (160 mg, 4.22 mmol) at 25° C. and stirred for 4 h. The reaction mixture diluted with ice water (30 mL) and extracted with EtOAc (3×50 mL). The organic layer was washed with brine (30 mL), dried over Na₂SO₄, and concentrated. The residue was purified by Combi-flash using 70% EtOAc in hexane to afford tert-butyl (R)-3-(4-(3-(hydroxymethyl) isoxazol-5-yl)-N-(8-methylisoquinolin-1-yl)piperidine-1-carboxamido)-piperidine-1-carboxylate (400 mg, 86%) as an off-white solid. LC-MS: [M+1⁺]=551.

### Step 5:

To a solution of tert-butyl (R)-3-(4-(3-(hydroxymethyl) isoxazol-5-yl)-N-(8-methylisoquinolin-1-yl)piperidine-1-carboxamido)piperidine-1-carboxylate (400 mg, 0.72 mmol) in DCM (10 mL) was added DMP (617 mg, 1.45 mmol) at 25° C. and stirred for 4 h. The reaction mixture diluted with ice water (50 mL) and extracted with DCM (3×100 mL). The combined organic layer was washed with brine (50 mL), dried over Na₂SO₄, and concentrated. The residue was purified by Combi-flash using 70% EtOAc in hexane to afford tert-butyl (R)-3-(4-(3-formylisoxazol-5-yl)-N-(8-methylisoquinolin-1-yl)piperidine-1-carboxamido)piperidine-1-carboxylate (250 mg, 62%) as an off-white solid.

### Step 6:

To a solution of tert-butyl (R)-3-(4-(3-formylisoxazol-5-yl)-N-(8-methylisoquinolin-1-yl)piperidine-1-carboxamido)piperidine-1-carboxylate (250 mg, 0.45 mmol) in DCE (3 mL) was added DAST (0.3 mL, 2.28 mmol) at 25° C. and the reaction mixture was stirred at 60° C. for 3 h. The reaction mixture was concentrated. The residue was purified by Combi-flash using 30% EtOAc in hexane to afford tert-butyl (R)-3-(4-(3-(difluoromethyl) isoxazol-5-yl)-N-(8-methylisoquinolin-1-yl)piperidine-1-carboxamido)piperidine-1-carboxylate (180 mg, 69%) as an off-white solid. LC-MS: [M+1⁺]=570.3. HPLC: 85.5%.

### Step 7:

To a solution of tert-butyl (R)-3-(4-(3-(difluoromethyl) isoxazol-5-yl)-N-(8-methylisoquinolin-1-yl)piperidine-1-carboxamido)piperidine-1-carboxylate (180 mg, 0.31 mmol) in DCM (1 mL) at 0° C. was added TFA (1 mL). The reaction mixture was stirred at 25° C. for 2 h before it was concentrated. The residue was purified by prep-HPLC (Conditions: Column: Luna OMEGA C-18 (250×21.2×5 μm), Mobile phase A: 0.1% TFA in H₂O, Mobile phase B: 100% MeCN, Gradient: T/% B: 0/30, 10/55. Flow: 15 mL/min, Dilution: MeCN+H₂O+THF). Pure fractions were dried by lyophilization to afford (R)-4-(3-(difluoromethyl) isoxazol-5-yl)-N-(8-methylisoquinolin-1-yl)-N-(piperidin-3-yl)piperidine-1-carboxamide trifluoroacetic acid salt (40 mg, 33%) as an off-white solid. LC-MS: [M+1+]=470.2. ¹H NMR (CD₃OD, 400 MHZ): δ ppm 8.38 (d, J=5.6 Hz, 1H), 7.85-7.81 (m, 1H), 7.75 (d, J=5.6 Hz, 1 h), 7.64-7.61 (m, 1H), 7.51 (d, J=6.8 Hz, 1H), 6.81 (t, J=53.6 Hz, 1H), 6.09 (s, 1H), 4.79-4.77 (m, 1H), 3.94-3.88 (m, 1H), 3.58-3.47 (m, 2H), 2.93-2.89 (m, 1H), 2.73 (m, 1H), 2.70 (s, 3H), 2.50-2.46 (m, 2H), 1.98 (m, 1H), 1.86-1.58 (m, 5H), 1.28-1.23 (m, 2H), 1.06-1.02 (m, 2H).

### Synthesis of (R)-2-fluoro-4-(4-methyl-1H-1,2,3-triazol-1-yl)-N-(8-methylisoquinolin-1-yl)-N-(piperidin-3-yl)benzamide (Example 251)

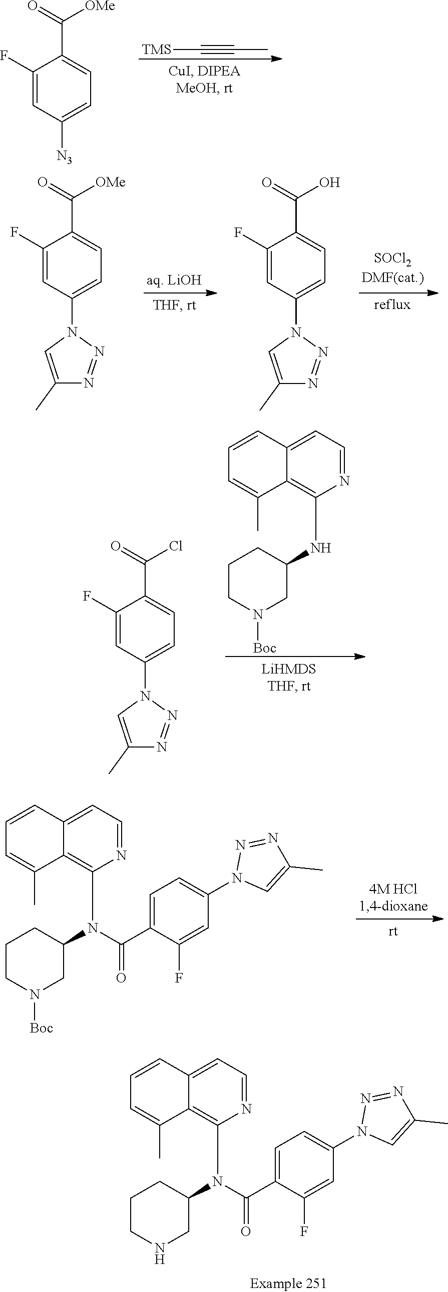

### Step 1:

To a mixture of methyl 4-azido-2-fluorobenzoate (1 g, 5.124 mmol) and trimethyl(prop-1-yn-1-yl) silane (2.27 mL, 15.372 mmol) in MeOH (30 mL) was added CuI (480 mg, 2.562 mmol) and DIPEA (4.6 mL, 25.621 mmol) at room temperature and stirred for 16 h. The reaction mixture was filtered through a pad of celite and rinsed with EtOAc. Then the filtrate was diluted with water (30 mL) and extracted with EtOAc (2×40 mL). The combined organic layer was washed with brine, dried over Na₂SO₄, and concentrated to afford methyl 2-fluoro-4-(4-methyl-1H-1,2,3-triazol-1-yl)benzoate (600 mg, 50%). LC-MS: [M+H⁺]=235.9.

### Step 2:

To a stirred solution of methyl 2-fluoro-4-(4-methyl-1H-1,2,3-triazol-1-yl)benzoate (450 mg, 1.913 mmol) in THF (4 mL) and water (1 mL) was added lithium hydroxide (100 mg, 4.166 mmol) at room temperature and stirred for 16 h. The reaction mixture was diluted with water, acidified with 1N aq. HCl, and extracted with EtOAc (2×50 mL). The combined organic layer was dried over Na₂SO₄ and concentrated to afford 2-fluoro-4-(4-methyl-1H-1,2,3-triazol-1-yl)benzoic acid (300 mg, 81%) as an off-white solid. LC-MS: [M+H⁺]=222.35.

### Step 3:

To the stirred solution of 2-fluoro-4-(4-methyl-1H-1,2,3-triazol-1-yl)benzoic acid (300 mg, 1.356 mmol) in SOCl₂ (15 mL) followed by the addition of DMF (0.01 mL) at room temperature and the reaction mixture was stirred at 80° C. for 16 h. The reaction mixture was concentrated and co-distilled with toluene to afford crude 2-fluoro-4-(4-methyl-1H-1,2,3-triazol-1-yl)benzoyl chloride (350 mg) as a brown solid.

### Step 4:

To the stirred solution of tert-butyl (R)-3-((8-methylisoquinolin-1-yl)amino)piperidine-1-carboxylate (250 mg, 0.732 mmol) in THF (4 mL) was added 1M LiHMDS in THF (1.7 mL) at 0° C. and stirred for 10 min and followed by addition of crude 2-fluoro-4-(4-methyl-1H-1,2,3-triazol-1-yl)benzoyl chloride (210 mg, 0.876 mmol) in THF (1 mL). The reaction mixture was stirred at room temperature for 1 h before it was diluted with water (50 mL) and extracted with EtOAc (2×50 mL). The combined organic layer was washed with brine solution, dried over Na₂SO₄, and concentrated. The residue was purified by Prep-HPLC (Conditions: Column: Luna OMEGA C-18 (250×21.2×5 μm), Mobile phase A: 0.1% formic acid in H₂O, Mobile phase B: 100% MeCN, Gradient: Time (min)/% B: 0/55, 10/85, Flow: 15 mL/min, Dilution: MeCN+H₂O+THF). Pure fractions were combined and dried by lyophilization to afford tert-butyl (R)-3-(2-fluoro-4-(4-methyl-1H-1,2,3-triazol-1-yl)-N-(8-methylisoquinolin-1-yl)benzamido)piperidine-1-carboxylate (60 mg, 20%) as an off-white solid. LC-MS: [M+H⁺]=544.7.

### Step 5:

The title compound hydrochloride salt was obtained as an off-white solid by deprotection Method A used in Example 97. LC-MS: [M+H⁺]=445.25. ¹H NMR (DMSO-d₆, 400 MHz) δ ppm 8.47-8.42 (m, 2H), 7.83-7.81 (m, 1H), 7.75-7.73 (m, 1H), 7.56-7.52 (m, 3H), 7.33-7.31 (m, 1H), 7.02-6.98 (m, 1H), 4.93-4.89 (m, 1H), 3.83-3.68 (m, 1H), 3.30-3.24 (m, 2H), 3.83-3.78 (m, 1H), 2.78 (s, 3H), 2.23 (s, 3H), 1.98-1.78 (m, 3H), 1.29-1.23 (m, 1H).

### Synthesis of (R)-4-(3H-[1,2,3]triazolo[4,5-b]pyridin-3-yl)-N-(4,8-dimethylisoquinolin-1-yl)-2-fluoro-N-(piperidin-3-yl)benzamide (Example 252)

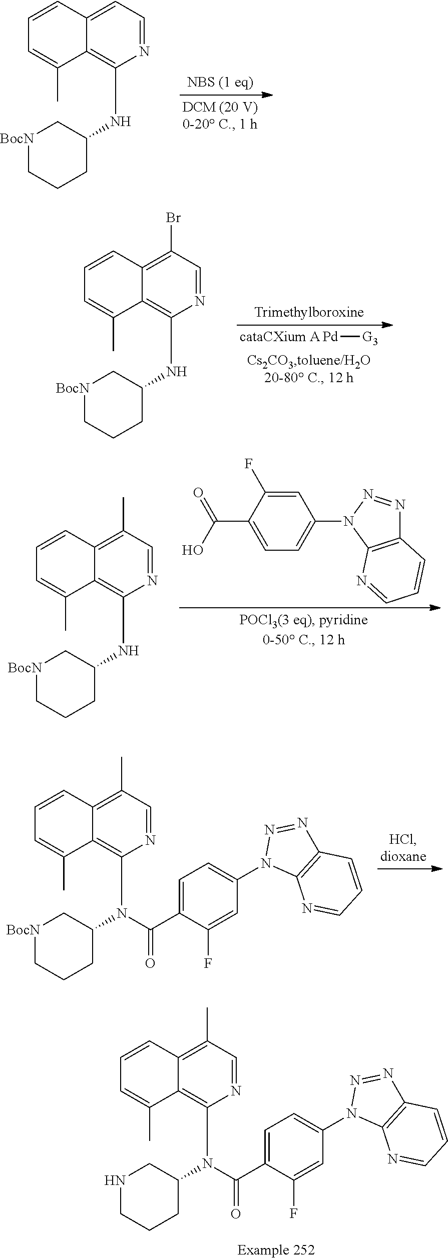

### Step 1:

To a solution of tert-butyl (R)-3-((8-methylisoquinolin-1-yl)amino)piperidine-1-carboxylate (0.35 g, 922.55 μmol) in DCM (7 mL) at 20° C. under N₂ atmosphere was added NBS (164.20 mg, 922.55 μmol). The mixture was stirred at 0° C. for 30 min and then at 20° C. for 30 min. The mixture was poured into NaHCO₃ aqueous (30 mL) and extracted with dichloromethane (3×30 mL). The combined organic phase was washed with brine (100 mL), dried over anhydrous Na₂SO₄, concentrated to give tert-butyl (R)-3-((4-bromo-8-methylisoquinolin-1-yl)amino)piperidine-1-carboxylate (0.4 g, 823%) as a brown oil. 1H NMR (CD₃OD, 400 MHZ) δ ppm 8.00 (s, 1H), 7.91 (br d, 1H, J=8.1 Hz), 7.58 (t, 1H, J=7.8 Hz), 7.38 (br d, 1H, J=7.1 Hz), 3.9-4.3 (m, 2H), 3.5-3.9 (m, 2H), 2.95 (s, 3H), 1.9-2.1 (m, 3H), 1.6-1.9 (m, 3H), 1.3-1.5 (m, 3H), 0.9-1.2 (m, 6H).

### Step 2:

To a solution of tert-butyl (R)-3-((4-bromo-8-methylisoquinolin-1-yl)amino)piperidine-1-carboxylate (0.520 g, 989.67 μmol), 2,4,6-trimethyl-1,3,5,2,4,6-trioxatriborinane in THF (3.5M, 848.29 μL) in water (2 mL) and toluene (8 mL) at 20° C. under N₂ atmosphere was added Cs₂CO₃ (644.91 mg, 1.98 mmol) and [2-(2-aminophenyl)phenyl]palladium (1+); bis(1-adamantyl)-butyl-phosphane; methanesulfonate (72.07 mg, 98.97 μmol). Then the mixture was stirred at 80° C. for 12 h before it was poured into water (50 mL). Then the mixture was extracted with EtOAc (3×50 mL). The organic phase was washed with brine (100 mL), dried over anhydrous Na₂SO₄, concentrated. The residue was purified by column chromatography (SiO₂, Petroleum ether/EtOAc=90/10) to give tert-butyl (R)-3-((4,8-dimethylisoquinolin-1-yl)amino)piperidine-1-carboxylate (0.4 g, 94%) as a brown oil. ¹H NMR (CD₃OD, 400 MHZ) δ ppm 7.6-7.7 (m, 2H), 7.53 (t, 1H, J=7.7 Hz), 7.32 (d, 1H, J=7.2 Hz), 4.0-4.2 (m, 2H), 3.7-3.9 (m, 1H), 3.5-3.7 (m, 1H), 3.1-3.2 (m, 1H), 2.38 (s, 3H), 1.9-2.1 (m, 4H), 1.7-1.9 (m, 2H), 1.65 (br d, 1H, J=8.5 Hz), 1.3-1.6 (m, 3H), 1.0-1.2 (m, 6H).

### Step 3:

To a solution of tert-butyl (R)-3-((4,8-dimethylisoquinolin-1-yl)amino)piperidine-1-carboxylate (200 mg, 478.24 μmol) and 4-(3H-[1,2,3]triazolo[4,5-b]pyridin-3-yl)-2-fluorobenzoic acid (411.61 mg, 1.43 mmol) in pyridine (2 mL) at 0° C. under N₂ atmosphere was added POCl₃ (219.99 mg, 1.43 mmol). Then the mixture was stirred at 50° C. for 12 h before it was poured into water (50 mL). Then the mixture was extracted with EtOAc (3×50 mL). The organic phase was washed with brine (100 mL), dried over anhydrous Na₂SO₄, concentrated. The residue was purified by prep-HPLC and the pure fraction was lyophilized to give tert-butyl (R)-3-(4-(3H-[1,2,3]triazolo[4,5-b]pyridin-3-yl)-N-(4,8-dimethylisoquinolin-1-yl)-2-fluorobenzamido)-piperidine-1-carboxylate (0.05 g, 13%) was obtained as a white solid. ¹H NMR (CDCl₃, 400 MHZ) δ ppm 8.72 (dd, 1H, J=1.4, 4.5 Hz), 8.41 (dd, 1H, J=1.4, 8.3 Hz), 8.2-8.3 (m, 1H), 8.0-8.1 (m, 1H), 7.83 (dd, 1H, J=1.8, 8.4 Hz), 7.66 (br d, 1H, J=8.0 Hz), 7.4-7.5 (m, 3H), 7.17 (t, 1H, J=7.9 Hz), 4.89 (br d, 1H, J=11.4 Hz), 4.5-4.7 (m, 1H), 3.9-4.3 (m, 2H), 3.55 (br t, 1H, J=11.4 Hz), 2.98 (br s, 1H), 2.90 (s, 2H), 2.6-2.7 (m, 1H), 2.55 (s, 3H), 1.7-1.9 (m, 1H), 1.64 (br d, 2H, J=1.9 Hz), 1.55 (s, 9H).

### Step 4:

The title compound hydrochloride salt was obtained as an off-white solid by deprotection Method A used in Example 97. LC-MS (ESI⁺): m/z 496.3 [M+H]⁺. ¹H NMR (DMSO-d₆, 400 MHz) δ ppm 9.2-9.4 (m, 1H), 8.9-9.2 (m, 1H), 8.8-8.8 (m, 1H), 8.68 (dd, 1H, J=1.4, 8.3 Hz), 8.3-8.5 (m, 1H), 7.9-8.0 (m, 1H), 7.8-7.8 (m, 2H), 7.5-7.7 (m, 3H), 7.1-7.2 (m, 1H), 4.9-5.0 (m, 1H), 3.8-3.9 (m, 2H), 3.4-3.6 (m, 2H), 3.2-3.3 (m, 1H), 2.8-2.9 (m, 3H), 2.52 (br s, 3H), 1.7-2.0 (m, 3H), 1.1-1.3 (m, 1H). ¹⁹F NMR (DMSO-d6, 377 MHz) δ ppm −111.6-−108.7 (m, 1F).

### Synthesis of (R)-4-(3H-[1,2,3]triazolo[4,5-b]pyridin-3-yl)-N-(5,8-dimethylisoquinolin-1-yl)-2-fluoro-N-(piperidin-3-yl)benzamide (Example 253)

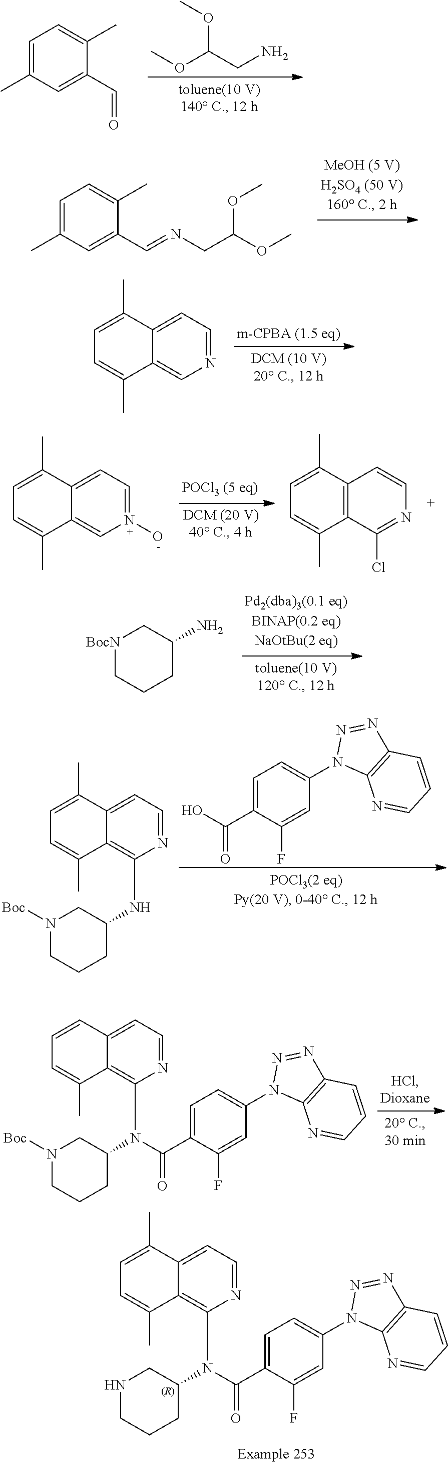

To a solution of compound 2,5-dimethylbenzaldehyde (13 g, 96.89 mmol) in toluene (130 mL) was added 2,2-dimethoxyethanamine (11.21 g, 106.58 mmol) at 20° C. The mixture was stirred at 140° C. for 12 h before it was concentrated to give crude (E)-N-(2,2-dimethoxyethyl)-1-(2,5 dimethylphenyl) methanimine (20 g, 89%) as a yellow oil. ¹H NMR (400 MHZ, CDCl₃) δ ppm 2.28 (s, 3H), 2.41 (s, 3H), 3.30 (s, 6H), 3.70 (dd, J=5.38, 1.25 Hz, 2H), 4.63 (t, J=5.38 Hz, 1H), 7.05-7.20 (m, 2H), 7.59 (s, 1H), 8.56 (s, 1H).

### Step 2:

A solution of compound (E)-N-(2,2-dimethoxyethyl)-1-(2,5 dimethylphenyl) methanimine (10 g, 42.93 mmol) in MeOH (50 mL) was added to H₂SO₄ (500 mL) dropwise over 15 min at 160° C. and the mixture was stirred for 2 h under N₂. The reaction mixture was quenched by addition of 50% aq. sodium hydroxide in water 300 mL at 20° C., and then extracted with EtOAc (2×100 mL). The combined organic layer was washed with brine (50 mL), dried over Na₂SO₄, filtered and concentrated to give compound 5,8-dimethylisoquinoline (6.5 g, 48%) as a yellow oil. ¹H NMR (400 MHZ, CDCl₃) δ ppm 2.64 (s, 3H), 2.76 (s, 3H), 7.28 (d, J=6.00 Hz, 1H), 7.40 (d, J=7.13 Hz, 1H), 7.75 (d, J=5.88 Hz, 1H), 8.59 (d, J=5.88 Hz, 1H), 9.45 (s, 1H).

### Step 3:

To a solution of compound 5,8-dimethylisoquinoline (4 g, 22.90 mmol) in DCM (40 mL) was added m-CPBA (7.41 g, 34.35 mmol) in portions at 20° C. The mixture was stirred at 20° C. for 12 h. The reaction mixture was quenched by addition aq. sodium thiosulfate (100 mL), and then extracted with dichloromethane (2×100 mL). The combined organic layer was washed with brine (100 mL), dried over Na₂SO₄, filtered and concentrated. The residue was purified by column chromatography (SiO₂, Petroleum ether/EtOAc=8/2) to give 5,8-dimethylisoquinoline 2-oxide (3 g, 68%) as light brown solid. ¹H NMR (400 MHZ, CDCl₃) δ ppm 2.61 (d, J=9.51 Hz, 6H), 7.31 (s, 2H), 7.80 (d, J=7.13 Hz, 1H), 8.19 (dd, J=7.25, 1.25 Hz, 1H), 8.95 (d, J=0.88 Hz, 1H).

### Step 4:

To a solution of compound 5,8-dimethylisoquinoline 2-oxide (2.8 g, 12.93 mmol, 90% purity, 1 eq) in DCM (56 mL) was added POCl₃ (9.91 g, 64.66 mmol, 6.03 mL, 5 eq) dropwise at 20° C. The mixture was stirred at 40° C. for 2 h. The reaction mixture was added to water (60 mL) at 30° C., then extracted with DCM (2×40 mL). The combined organic layer was washed with brine (30 mL), dried over Na₂SO₄, filtered and concentrated. The residue was purified by column chromatography (SiO₂, Petroleum ether/EtOAc=5/1) to give 1-chloro-5,8-dimethylisoquinoline (1 g, 3.39 mmol, 26.23% yield, 65% purity) as yellow solid. ¹H NMR (400 MHZ, DMSO-d₆) δ ppm 2.59 (s, 3H), 2.94 (s, 3H), 7.44-7.49 (m, 1H), 7.53-7.58 (m, 1H), 7.87 (d, J=5.75 Hz, 1H), 8.27 (d, J=5.75 Hz, 1H).

### Step 5:

To a solution of compound 1-chloro-5,8-dimethylisoquinoline (0.9 g, 4.23 mmol, 65% purity, 1 eq) in toluene (9 mL) was added tert-butyl (R)-3-aminopiperidine-1-carboxylate (1.02 g, 5.07 mmol, 1.2 eq), BINAP (526.32 mg, 845.26 μmol, 0.2 eq), t-BuONa (812.33 mg, 8.45 mmol, 2 eq) and Pd₂(dba)₃ (387.01 mg, 422.63 μmol, 0.1 eq) under N₂ at 20° C. The mixture was stirred at 120° C. for 12 h under N₂. The reaction mixture was partitioned between water (30 mL) and EtOAc (30 mL). The organic phase was separated, washed with brine (30 mL), dried over Na₂SO₄, filtered and concentrated, which was purified by column chromatography (SiO₂, Petroleum ether/EtOAc=3/1) to give tert-butyl (R)-3-((5,8-dimethylisoquinolin-1-yl)amino)piperidine-1-carboxylate (0.7 g, 1.77 mmol, 41.93% yield, 90% purity) as a yellow solid. ¹H NMR (400 MHZ, CDCl₃) δ ppm 1.22-1.52 (m, 9H), 1.58-1.93 (m, 4H), 2.53 (s, 3H), 2.89 (s, 3H), 3.15 (ddd, J=13.10, 9.72, 3.44 Hz, 1H), 3.46 (dd, J=13.26, 2.75 Hz, 1H), 3.73-3.97 (m, 2H), 4.39 (br s, 1H), 5.80 (br d, J=1.25 Hz, 1H), 6.98 (d, J=6.00 Hz, 1H), 7.08 (d, J=7.13 Hz, 1H), 7.25 (br d, J=7.50 Hz, 1H), 7.97 (d, J=5.88 Hz, 1H).

### Step 6:

To a solution of 4-(3H-[1,2,3]triazolo[4,5-b]pyridin-3-yl)-2-fluorobenzoic acid (163.44 mg, 569.66 μmol) and compound tert-butyl (R)-3-((5,8-dimethylisoquinolin-1-yl)amino)piperidine-1-carboxylate (150.00 mg, 379.78 μmol) in pyridine (1 mL) was added POCl₃ (116.46 mg, 759.55 μmol, 70.80 μL, 2 eq) at 20° C. The mixture was stirred at 40° C. for 12 h before it was concentrated. The residue was diluted with water 10 mL and extracted with EtOAc (2×10 mL). The combined organic layer was washed with brine (20 mL), dried over Na₂SO₄, filtered and concentrated. The residue was purified by prep-HPLC (column: Phenomenex luna C18 100*40 mm*5 μm; mobile phase: [H₂O (0.04% HCl)-MeCN]; gradient: 50%-80% B over 8.0 min) and lyophilized to give tert-butyl (R)-3-(4-(3H-[1,2,3]triazolo[4,5-b]pyridin-3-yl)-N-(5,8-dimethylisoquinolin-1-yl)-2-fluorobenzamido)piperidine-1-carboxylate (0.04 g, 14.15% yield) as light yellow solid. 1H NMR (400 MHZ, CDCl₃) δ ppm 1.20-1.20 (m, 1H), 1.28 (br d, J=4.50 Hz, 1H), 1.41 (br s, 3H), 1.55 (s, 7H), 1.62-1.72 (m, 3H), 2.52 (s, 3H), 2.87 (s, 3H), 3.52-3.76 (m, 1H), 3.86-4.37 (m, 2H), 4.54-4.78 (m, 1H), 4.87-5.05 (m, 1H), 7.36 (s, 2H), 7.42 (dd, J=8.32, 4.57 Hz, 1H), 7.73-7.82 (m, 1H), 7.86-7.94 (m, 1H), 7.96-8.08 (m, 1H), 8.41 (dd, J=8.25, 1.38 Hz, 1H), 8.46-8.54 (m, 1H), 8.72 (dd, J=4.50, 1.38 Hz, 1H).

### Step 7:

The title compound hydrochloride salt (14 mg, 48%) as a white solid was prepared according to the deprotection procedure described for Example 241. LC-MS (ESI⁺): m/z 496.2 [M+H]⁺; ¹H NMR (CD₃OD, 400 MHZ): δ ppm 8.66 (d, J=3.6 Hz, 1H), 8.42-8.39 (m, 2H), 7.93-7.90 (m, 1H), 7.80 (d, J=6.0 Hz, 1H), 7.72-7.71 (m, 1H), 7.45-7.42 (m, 1H), 7.29-7.22 (m, 2H), 7.01-7.95 (m, 1H), 5.01-4.95 (m, 1H), 3.95-3.92 (m, 1H), 3.77-3.71 (m, 1H), 3.36-3.29 (m, 1H), 2.95-2.91 (m, 1H), 2.73 (s, 3H), 2.52 (m, 1H), 2.38 (s, 3H), 1.88-1.82 (m, 2H), 1.52-1.46 (m, 1H). ¹⁹F NMR (377 MHz, CD₃OD) δ ppm −110.29 (s, 1F).

### Synthesis of (R)-4-(3H-[1,2,3]triazolo[4,5-b]pyridin-3-yl)-N-(6,8-dimethylisoquinolin-1-yl)-2-fluoro-N-(piperidin-3-yl)benzamide (Example 254)

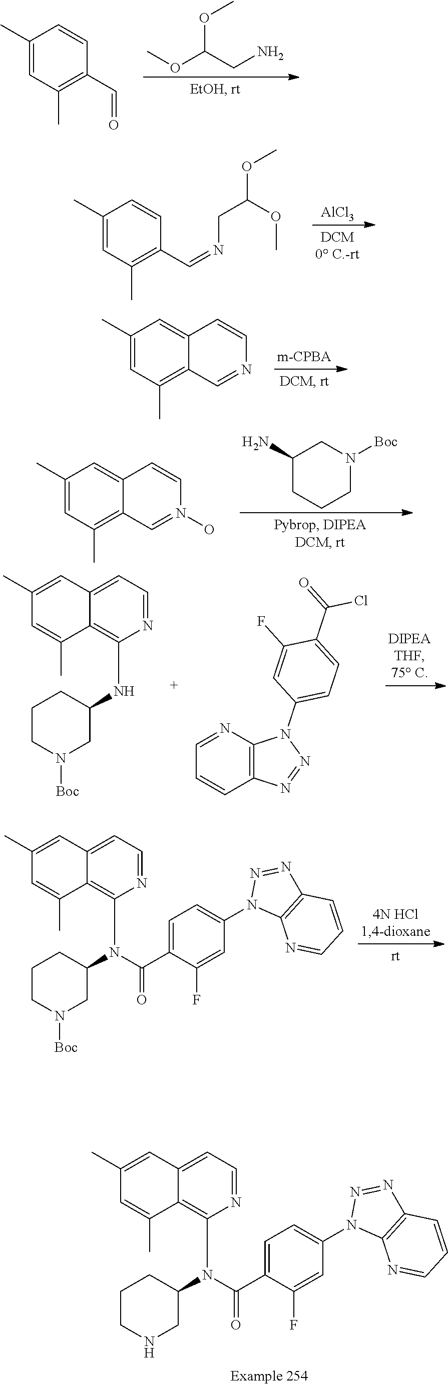

Example 254 was prepared according to the procedure described for Example 253 by substituting 2,5-dimethylbenzaldehyde with 2,4-dimethylbenzaldehyde, provided the title compound hydrochloride salt as an off-white solid. LC-MS: [M+H⁺]=496.2. ¹H NMR (CD₃OD, 400 MHZ): δ ppm 8.67 (d, J=3.2 Hz, 1H), 8.41 (dd, J=1.2, 8.0 Hz, 1H), 8.31-8.29 (m, 1H), 7.96-7.93 (m, 1H), 7.77-7.74 (m, 1H), 7.55 (d, J=5.6 Hz, 1H), 7.44 (dd, J=4.8, 8.4 Hz, 1H), 7.33 (m, 1H), 7.27 (m, 1H), 7.04-6.99 (m, 1H), 5.00-4.94 (m, 1H), 3.94-3.90 (m, 1H), 3.75-3.69 (m, 1H), 3.32-3.29 (m, 1H), 2.93-2.90 (m, 1H), 2.74 (s, 3H), 2.64-2.62 (m, 1H), 2.26 (s, 3H), 1.86-1.81 (m, 3H).

### Synthesis of (R)-4-(3H-[1,2,3]triazolo[4,5-b]pyridin-3-yl)-N-(1,3-dimethyl-1H-pyrrolo[2,3-c]pyridin-7-yl)-2-fluoro-N-(piperidin-3-yl)benzamide (Example 255)

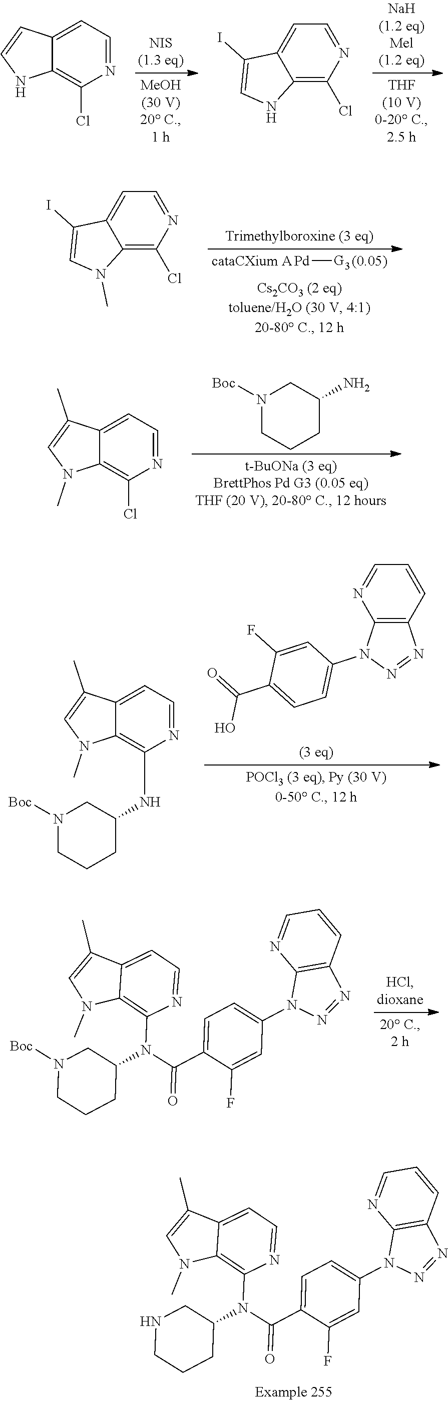

### Step 1:

To a solution of 7-chloro-1H-pyrrolo[2,3-c]pyridine (1 g, 6.55 mmol, 1 eq) in anhydrous MeOH (30 mL) was added 1-iodopyrrolidine-2,5-dione (1.92 g, 8.52 mmol, 1.3 eq) at 20° C. under N₂. Then the mixture was stirred at 20° C. for 1 h. The reaction mixture was concentrated in vacuum and the mixture was poured into water (50 mL). Then the mixture was extracted with EtOAc (3×50 mL). The organic phase was washed with brine (100 mL), dried over anhydrous Na₂SO₄, concentrated in vacuum to give 7-chloro-3-iodo-1H-pyrrolo[2,3-c]pyridine (1.9 g, 93%) as off white solid. LC-MS: [M+H⁺]=278.8; 1H NMR (DMSO-d6, 400 MHZ): δ ppm=12.50 (br s, 1H), 8.00 (d, 1H, J=5.5 Hz), 7.90 (s, 1H), 7.32 (d, 1H, J=5.4 Hz).

### Step 2:

To a solution of 7-chloro-3-iodo-1H-pyrrolo[2,3-c]pyridine (1.9 g, 6.14 mmol, purity 90%, 1 eq) in THF (40 mL) was added NaH (294.75 mg, 7.37 mmol, 60% purity, 1.2 eq) in portions at 0° C. under N₂. After addition, the mixture was stirred at 0° C. for 30 min, and then Mel (1.05 g, 7.37 mmol, 1.2 eq) was added dropwise at 0° C. under N₂. Then the resulting mixture was stirred at 20° C. for 2 h. The reaction mixture was quenched with ammonium chloride aqueous solution (50 mL) at 0° C. Then the mixture was extracted with EtOAc (3×50 mL). The organic phase was washed with brine (100 mL), dried over anhydrous Na₂SO₄, concentrated in vacuum to give a residue, which was purified by column chromatography (SiO₂, petroleum ether/EtOAc=95/5) to give 7-chloro-3-iodo-1-methyl-1H-pyrrolo[2,3-c]pyridine (1.3 g, 65%) as light yellow solid. LC-MS: [M+H⁺]=292.8. ¹H NMR (DMSO-d6, 400 MHZ): δ ppm 7.98 (d, 1H, J=5.4 Hz), 7.89 (s, 1H), 7.30 (d, 1H, J=5.4 Hz), 4.13 (s, 3H).

### Step 3:

To a solution of 7-chloro-3-iodo-1-methyl-1H-pyrrolo[2,3-c]pyridine (1.2 g, 3.69 mmol, purity 90%, 1 eq), 2,4,6-trimethyl-1,3,5,2,4,6-trioxatriborinane (3.5 M, 3.16 mL, 3 eq) in water (7 mL) and toluene (28 mL) was added Cs₂CO₃ (2.41 g, 7.38 mmol, 2 eq) and [2-(2-aminophenyl)phenyl]palladium (1+); bis(1-adamantyl)-butyl-phosphane; methanesulfonate (268.90 mg, 369.23 μmol, 0.1 eq) in order at 20° C. under N₂. Then the mixture was stirred at 80° C. for 12 h. One addition vial in 100 mg scale was set up as described above. These two reactions were combined. The mixture was poured into water (20 mL). Then the mixture was extracted with EtOAc (3×30 mL). The organic phase was washed with brine (100 mL), dried over anhydrous Na₂SO₄, concentrated in vacuum to give a residue, which was purified by column chromatography (SiO₂, Petroleum ether/EtOAc=95/5) to give 7-chloro-1,3-dimethyl-1H-pyrrolo[2,3-c]pyridine (0.5 g, 62%) as brown solid. LC-MS: [M+H⁺]=180.9.

¹H NMR (DMSO-d6, 400 MHZ): δ ppm 7.86 (d, 1H, J=5.3 Hz), 7.51 (d, 1H, J=5.4 Hz), 7.41 (s, 1H), 4.06 (s, 3H), 2.23 (d, 3H, J=0.8 Hz).

### Steps 4-6:

The title compound hydrochloride salt as a white solid was prepared according to the deprotection procedure described for Example 253. LC-MS: [M+H⁺]=485.2. ¹H NMR (CD₃OD, 400 MHZ): δ ppm 8.78 (br d, 1H, J=3.4 Hz), 8.53 (br d, 1H, J=7.4 Hz), 8.1-8.2 (m, 2H), 8.0-8.1 (m, 1H), 7.6-7.8 (m, 2H), 7.4-7.6 (m, 3H), 5.0-5.1 (m, 2H), 4.12 (br s, 1H), 4.04 (s, 3H), 3.70 (br t, 1H, J=11.6 Hz), 3.38 (br d, 2H, J=12.9 Hz), 2.9-3.0 (m, 1H), 2.20 (s, 3H), 1.91 (br s, 3H), 1.3-1.4 (m, 1H).

### Synthesis of N-(isoquinolin-3-yl)-4-(1-methylpyrrolidin-3-yl)-N—((R)-piperidin-3-yl)piperidine-1-carboxamide (Example 256)

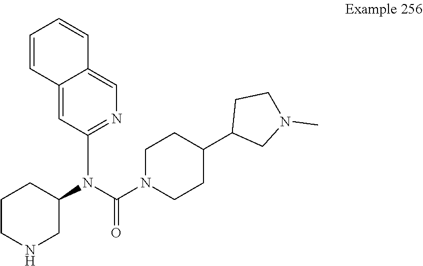 [SMILES: CN1CCC(C2CCN(C(=O)N(c3cc4ccccc4cn3)[C@@H]3CCCNC3)CC2)C1]

Example 256 was prepared according to the procedure described for Example 242 by substituting tert-butyl (3R)-3-[chlorocarbonyl-(8-methyl-1-isoquinolyl)amino]piperidine-1-carboxylate and 3-methyl-5-(4-piperidyl) isoxazole with tert-butyl (R)-3-((chlorocarbonyl)(isoquinolin-3-yl)amino)piperidine-1-carboxylate and 4-(1-methylpyrrolidin-3-yl)piperidine, provided the title compound trifluoroacetic acid salt as a solid. LC-MS: [M+H⁺]=422.

### Synthesis of (R)-4-(6-fluorobenzo[d]isoxazol-3-yl)-N-(8-methylisoquinolin-1-yl)-N-(piperidin-3-yl)-3,6-dihydropyridine-1(2H)-carboxamide (Example 257)

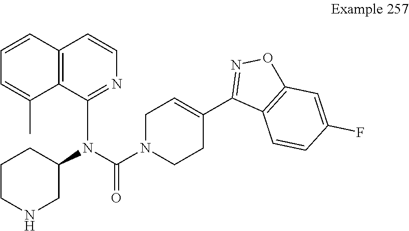 [SMILES: Cc1cccc2ccnc(N(C(=O)N3CC=C(c4noc5cc(F)ccc45)CC3)[C@@H]3CCCNC3)c12]

Example 257 was prepared according to the procedure described for Example 287 by substituting 4-phenyl-1,2,3,6-tetrahydropyridine with 6-fluoro-3-(1,2,3,6-tetrahydropyridin-4-yl)benzo[d]isoxazole, providing the title compound trifluoroacetic acid salt. LC-MS: [M+H⁺]=486.

### Synthesis of N²,N²-dimethyl-N¹-(8-methylisoquinolin-1-yl)-N1-((R)-piperidin-3-yl)piperidine-1,2-dicarboxamide (Example 258)

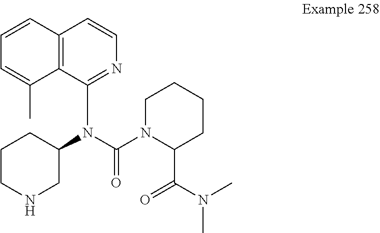 [SMILES: Cc1cccc2ccnc(N(C(=O)N3CCCCC3C(=O)N(C)C)[C@@H]3CCCNC3)c12]

Example 258 was prepared according to the procedure described for Example 287 by substituting 4-phenyl-1,2,3,6-tetrahydropyridine with N,N-dimethylpiperidine-2-carboxamide and heating the reaction in step 3 at 100° C. for 16 h, provided the title compound trifluoroacetic acid salt as a solid. LC-MS: [M+H⁺]=424.

### Synthesis of 2-(benzo[d]thiazol-2-yl)-N-(8-methylisoquinolin-1-yl)-N—((R)-piperidin-3-yl)piperidine-1-carboxamide (Example 259)

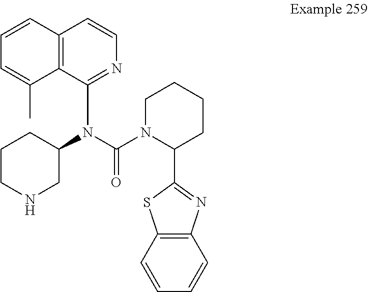 [SMILES: Cc1cccc2ccnc(N(C(=O)N3CCCCC3c3nc4ccccc4s3)[C@@H]3CCCNC3)c12]

Example 259 was prepared according to the procedure described for Example 287 by substituting 4-phenyl-1,2,3,6-tetrahydropyridine with 2-(piperidin-2-yl)benzo[d]thiazole, provided the title compound trifluoroacetic acid salt. LC-MS: [M+H⁺]=486.2.

### Synthesis of 3-(cyclobutylmethyl)-N-(8-methylisoquinolin-1-yl)-N—((R)-piperidin-3-yl)piperidine-1-carboxamide (Example 260)

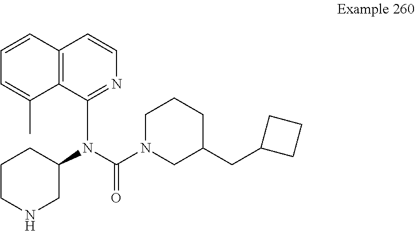 [SMILES: Cc1cccc2ccnc(N(C(=O)N3CCCC(CC4CCC4)C3)[C@@H]3CCCNC3)c12]

Example 260 was prepared according to the procedure described for Example 287 by substituting 4-phenyl-1,2,3,6-tetrahydropyridine with 3-(cyclobutylmethyl)piperidine, provided the title compound trifluoroacetic acid salt as a solid. LC-MS: [M+H⁺]=421.3.

### Synthesis of 4-cyclobutyl-N-(8-methylisoquinolin-1-yl)-N—((R)-piperidin-3-yl)azepane-1-carboxamide (Example 261)

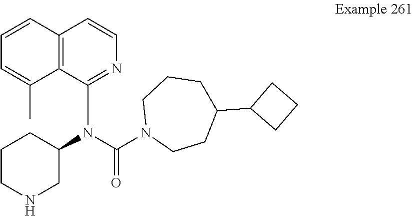 [SMILES: Cc1cccc2ccnc(N(C(=O)N3CCCC(C4CCC4)CC3)[C@@H]3CCCNC3)c12]

Example 261 was prepared according to the procedure described for Example 287 by substituting 4-phenyl-1,2,3,6-tetrahydropyridine with 4-cyclobutylazepane, providing the title compound trifluoroacetic acid salt. LC-MS: [M+H⁺]=421.

### Synthesis of (R)-4-(3-cyclopropyl-1,2,4-oxadiazol-5-yl)-N-(8-methylisoquinolin-1-yl)-N-(piperidin-3-yl)piperidine-1-carboxamide (Example 262)

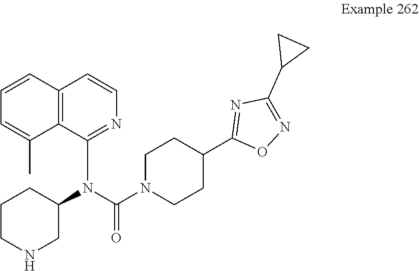 [SMILES: Cc1cccc2ccnc(N(C(=O)N3CCC(c4nc(C5CC5)no4)CC3)[C@@H]3CCCNC3)c12]

Example 262 was prepared according to the procedure described for Example 287 by substituting 4-phenyl-1,2,3,6-tetrahydropyridine with 3-cyclopropyl-5-(piperidin-4-yl)-1,2,4-oxadiazole, provided the title compound trifluoroacetic acid salt. LC-MS: [M+H⁺]=461.

### Synthesis of (R)-3-(N-(8-methylisoquinolin-1-yl)-4-(3-phenyl-1,2,4-oxadiazol-5-yl)piperidine-1-carboxamido)piperidine-1-carboxylate (Example 263)

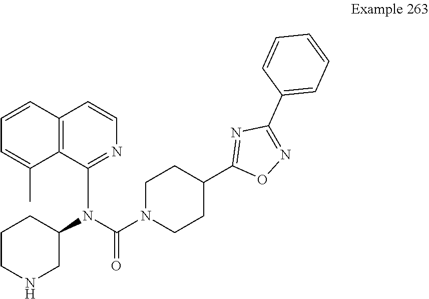 [SMILES: Cc1cccc2ccnc(N(C(=O)N3CCC(c4nc(-c5ccccc5)no4)CC3)[C@@H]3CCCNC3)c12]

Example 263 was prepared according to the procedure described for Example 287 by substituting 4-phenyl-1,2,3,6-tetrahydropyridine with 3-phenyl-5-(piperidin-4-yl)-1,2,4-oxadiazole, provided the title compound trifluoroacetic acid salt. LC-MS: [M+H⁺]=497.

### Synthesis of (R)-4-(benzo[d]isothiazol-3-yl)-N-(8-methylisoquinolin-1-yl)-N-(piperidin-3-yl)piperazine-1-carboxamide (Example 264)

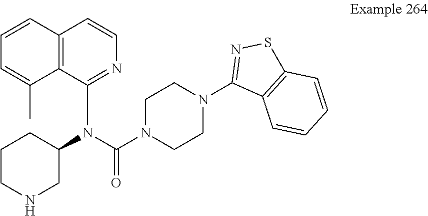 [SMILES: Cc1cccc2ccnc(N(C(=O)N3CCN(c4nsc5ccccc45)CC3)[C@@H]3CCCNC3)c12]

Example 264 was prepared according to the procedure described for Example 287 by substituting 4-phenyl-1,2,3,6-tetrahydropyridine with 3-(piperazin-1-yl)benzo[d]isothiazole, provided the title compound trifluoroacetic acid salt. LC-MS: [M+H⁺]=487.

### Synthesis of (R)—N-(8-methylisoquinolin-1-yl)-N-(piperidin-3-yl)-4′,5′-dihydrospiro[piperidine-4,7′-thieno[2,3-c]pyran]-1-carboxamide (Example 265)

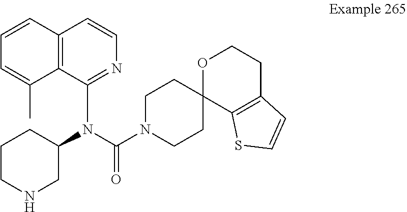 [SMILES: Cc1cccc2ccnc(N(C(=O)N3CCC4(CC3)OCCc3ccsc34)[C@@H]3CCCNC3)c12]

Example 265 was prepared according to the procedure described for Example 287 by substituting 4-phenyl-1,2,3,6-tetrahydropyridine with 4′,5′-dihydrospiro[piperidine-4,7′-thieno[2,3-c]pyran], provided the title compound trifluoroacetic acid salt. LC-MS: [M+H⁺]=477.

### Synthesis of (R)-4-(1H-indazol-1-yl)-N-(8-methylisoquinolin-1-yl)-N-(piperidin-3-yl)piperidine-1-carboxamide (Example 266)

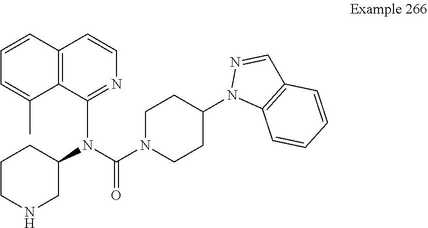 [SMILES: Cc1cccc2ccnc(N(C(=O)N3CCC(n4ncc5ccccc54)CC3)[C@@H]3CCCNC3)c12]

Example 266 was prepared according to the procedure described for Example 287 by substituting 4-phenyl-1,2,3,6-tetrahydropyridine with 1-(piperidin-4-yl)-1H-indazole, provided the title compound trifluoroacetic acid salt. LC-MS: [M+H⁺]=469.

### Synthesis of 3-methyl-N-(8-methylisoquinolin-1-yl)-3-phenyl-N—((R)-piperidin-3-yl)piperidine-1-carboxamide (Example 267)

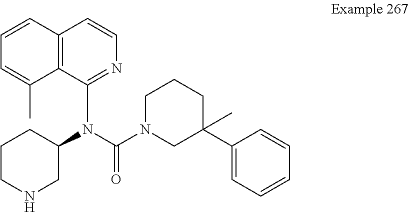 [SMILES: Cc1cccc2ccnc(N(C(=O)N3CCCC(C)(c4ccccc4)C3)[C@@H]3CCCNC3)c12]

Example 267 was prepared according to the procedure described for Example 287 by substituting 4-phenyl-1,2,3,6-tetrahydropyridine with 3-methyl-3-phenylpiperidine, providing the title compound trifluoroacetic acid salt. LC-MS: [M+H⁺]=443.

### Synthesis of (R)—N-(8-methylisoquinolin-1-yl)-4-(2-oxoquinolin-1(2H)-yl)-N-(piperidin-3-yl)piperidine-1-carboxamide (Example 268)

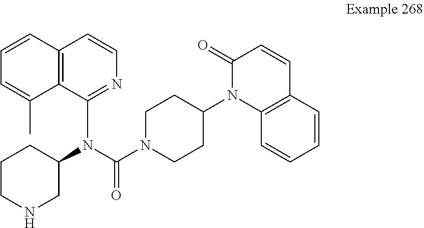 [SMILES: Cc1cccc2ccnc(N(C(=O)N3CCC(n4c(=O)ccc5ccccc54)CC3)[C@@H]3CCCNC3)c12]

Example 268 was prepared according to the procedure described for Example 287 by substituting 4-phenyl-1,2,3,6-tetrahydropyridine with 1-(piperidin-4-yl)quinolin-2 (1H)-one, provided the title compound trifluoroacetic acid salt. LC-MS: [M+H⁺]=496.

### Synthesis of (R)-4-(3-hydroxyphenyl)-N-(8-methylisoquinolin-1-yl)-N-(piperidin-3-yl)piperidine-1-carboxamide (Example 269)

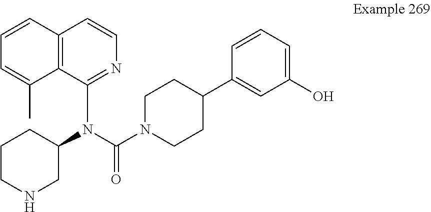 [SMILES: Cc1cccc2ccnc(N(C(=O)N3CCC(c4cccc(O)c4)CC3)[C@@H]3CCCNC3)c12]

Example 269 was prepared according to the procedure described for Example 287 by substituting 4-phenyl-1,2,3,6-tetrahydropyridine with 3-(piperidin-4-yl)phenol, provided the title compound trifluoroacetic acid salt. LC-MS: [M+H⁺]=445.

### Synthesis of (R)—N-(8-methylisoquinolin-1-yl)-4-(phthalazin-1-yl)-N-(piperidin-3-yl)piperidine-1-carboxamide (Example 270)

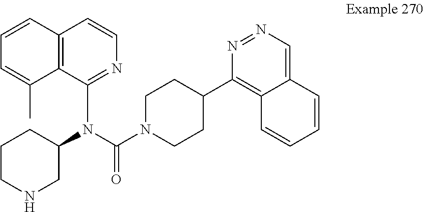 [SMILES: Cc1cccc2ccnc(N(C(=O)N3CCC(c4nncc5ccccc45)CC3)[C@@H]3CCCNC3)c12]

Example 270 was prepared according to the procedure described for Example 287 by substituting 4-phenyl-1,2,3,6-tetrahydropyridine with 1-(piperazin-1-yl)phthalazine, providing the title compound trifluoroacetic acid salt salt as an off-white solid. LC-MS: [M+H⁺]=482.

### Synthesis of (R)-3-((1H-1,2,3-triazol-1-yl)methyl)-2-fluoro-N-(8-methylisoquinolin-1-yl)-N-(piperidin-3-yl)benzamide (Example 271)

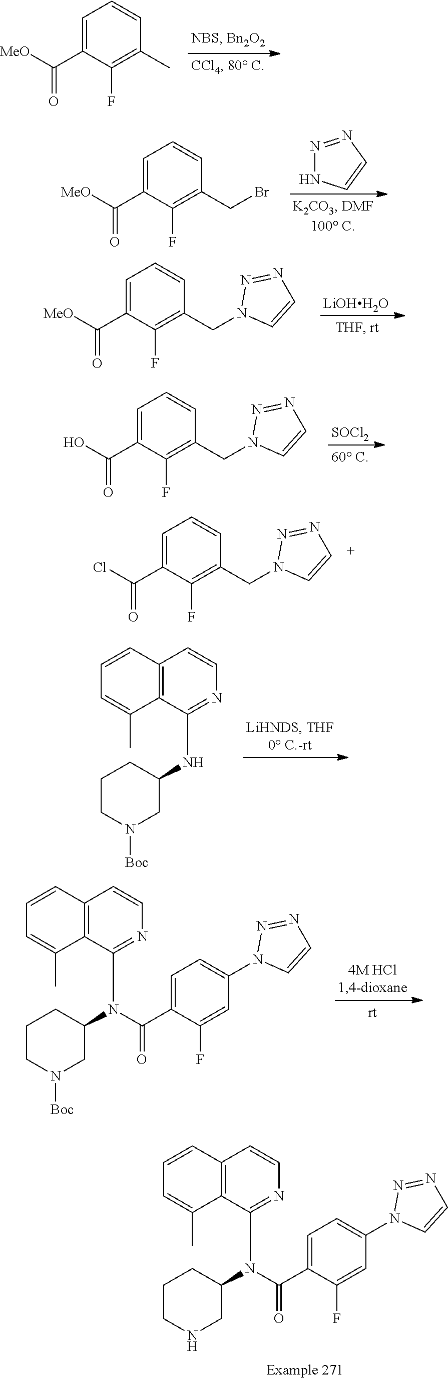

### Step 1:

To a solution of methyl 2-fluoro-3-methylbenzoate (3 g, 17.839 mmol) in CCl₄ (40 mL) at 0° C. was added NBS (2.52 g, 21.406 mmol) and AIBN (290 mg, 1.783 mmol). The reaction mixture was stirred at 80° C. for 16 h. The reaction mixture filtered, washed with ice cold water, filtrate was extracted with EtOAc (2×100 mL). The organic layer was washed with brine, dried over Na₂SO₄, and concentrated. The residue was purified by Combi-flash using 20% EtOAc in hexane to afford methyl 3-(bromomethyl)-2-fluorobenzoate (3 g, 68%) as a brownish liquid.

### Step 2:

To a solution of methyl 3-(bromomethyl)-2-fluorobenzoate (2.8 g, 11.333 mmol) in DMF (30 mL) at 0° C. was added 1H-1,2,3-triazole (0.8 mL, 13.599 mmol), K₂CO₃ (3.1 g, 22.666 mmol) and the reaction mixture was stirred at 100° C. for 48 h. The reaction mixture poured in water and extracted with EtOAc (2×100 mL). The organic layer was washed with brine, dried over Na₂SO₄, and concentrated. The residue was purified by Combi-flash using 50% EtOAc in hexane to afford methyl 3-((1H-1,2,3-triazol-1-yl)methyl)-2-fluorobenzoate (380 mg, 14%) as a light brown solid. LC-MS: [M+1⁺]=236.

### Step 3:

To a solution of methyl 3-((1H-1,2,3-triazol-1-yl)methyl)-2-fluorobenzoate (380 mg, 1.690 mmol) in THF (5 mL) and H₂O (5 mL) at 25° C. was added LiOH·H₂O (350 mg, 8.492 mmol). The reaction mixture stirred for 16 h before it was concentrated. The residue was diluted with water, acidified with 2N aq. HCl. The precipitate was collected and dried to afford 3-((1H-1,2,3-triazol-1-yl)methyl)-2-fluorobenzoic acid (300 mg, impure) as an off-white solid. LC-MS: [M+1+]=222.

### Step 4:

To a solution of 3-((1H-1,2,3-triazol-1-yl)methyl)-2-fluorobenzoic acid (300 mg, 1.357 mmol) in SOCl₂ (10 mL) was added DMF (0.1 mL) at 0° C. and the reaction mixture stirred at 80° C. for 16 h. The reaction mixture was concentrated to afford crude 3-((1H-1,2,3-triazol-1-yl)methyl)-2-fluorobenzoyl chloride (300 mg).

### Step 5:

To a solution of tert-butyl (R)-3-((8-methylisoquinolin-1-yl)amino)piperidine-1-carboxylate (200 mg, 0.585 mmol) in THF (10 mL) was added 1M LiHMDS in THF (1.75 mL, 1.757 mmol) at 0° C. and stirred for 10 min and followed by addition of crude 3-((1H-1,2,3-triazol-1-yl)methyl)-2-fluorobenzoyl chloride (160 mg, 0.702 mmol) in THF (5 mL). The reaction mixture was stirred at 0° C. for 10 min before it was quenched with ice cold water and extracted with EtOAc (2×50 mL). The organic layer was washed with brine, dried over Na₂SO₄, and concentrated. The residue was purified by Combi-flash using 40% EtOAc in first and then further purified by Prep-HPLC (Conditions: Column: Luna OMEGA C-18 (250×21.2×5 μm), Mobile phase A: 10 mM ammonium acetate in H₂O, Mobile phase B: MeCN (100%), Gradient: T/% B: 0/60, 10/80, Flow: 15 mL/min, Dilution: MeCN+H₂O+THF). Pure fractions were dried by lyophilization to afford tert-butyl (R)-3-(3-((1H-1,2,3-triazol-1-yl)methyl)-2-fluoro-N-(8-methylisoquinolin-1-yl)benzamido)piperidine-1-carboxylate (30 mg, 10%) as an off-white solid. LC-MS: [M+1+]=545.3.

### Step 6:

The title compound hydrochloride salt was obtained as an off-white solid by deprotection Method A used in Example 97. LC-MS: [M+1⁺]=445.95. ¹H NMR (CD₃OD, 400 MHZ): δ ppm 8.33-8.30 (m, 1H), 8.04-8.03 (m, 1H), 7.94 (s, 1H), 7.65-7.55 (m, 2H), 7.48-7.41 (m, 1H), 7.38-7.34 (m, 1H), 7.16-7.12 (m, 1H), 6.88-6.83 (m, 1H), 6.74-6.70 (m, 1H), 5.51 (s, 2H), 5.06-4.99 (m, 1H), 3.99-3.96 (m, 1H), 3.82-3.79 (m, 1H), 3.43-3.37 (m, 1H), 3.02-2.98 (m, 1H), 2.77 (s, 3H), 2.70-2.69 (m, 1H), 1.96-1.90 (m, 2H), 1.61-1.57 (m, 1H).

### Synthesis of (R)—N-(8-chloroisoquinolin-1-yl)-2-fluoro-N-(piperidin-3-yl)-4-(pyrimidin-2-ylamino)benzamide (Example 272)

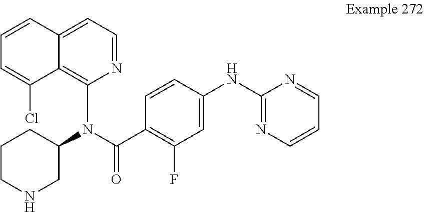 [SMILES: O=C(c1ccc(Nc2ncccn2)cc1F)N(c1nccc2cccc(Cl)c12)[C@@H]1CCCNC1]

Example 272 was prepared according to the procedure described for Example 119 by substituting 1-chloro-8-methylisoquinoline with 1,8-dichloroisoquinoline, provided the title compound hydrochloride salt as a white solid. LCMS (ESI⁺): m/z 477.1 (M+H. ¹H NMR (400 MHZ, DMSO-d₆) δ ppm 1.10-1.30 (m, 1H) 1.80 (br s, 3H) 1.96 (br d, J=12.76 Hz, 1H) 2.31-2.46 (m, 1H) 2.65-2.90 (m, 1H) 3.16-3.34 (m, 1H) 3.48 (q, J=11.26 Hz, 1H) 3.64-3.78 (m, 1H) 4.85-5.04 (m, 1H) 6.81 (t, J=8.32 Hz, 1H) 6.89 (t, J=4.82 Hz, 1H) 7.00 (dd, J=8.69, 1.56 Hz, 1H) 7.40-7.50 (m, 1H) 7.58-7.65 (m, 1H) 7.78 (d, J=7.00 Hz, 1H) 7.84-7.97 (m, 2H) 8.40-8.68 (m, 3H) 9.13 (br d, J=9.13 Hz, 1H) 9.33-9.56 (m, 1H) 9.81 (s, 1H).

### Synthesis of (R)-3-((2H-[1,2,3]triazolo[4,5-b]pyridin-2-yl)methyl)-2-fluoro-N-(8-methylisoquinolin-1-yl)-N-(piperidin-3-yl)benzamide (Example 273)

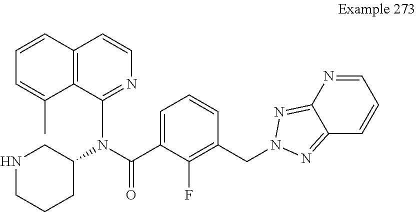 [SMILES: Cc1cccc2ccnc(N(C(=O)c3cccc(Cn4nc5cccnc5n4)c3F)[C@@H]3CCCNC3)c12]

Example 273 was prepared according to the procedure for Example 271 by substituting 3-((1H-1,2,3-triazol-1-yl)methyl)-2-fluorobenzoic acid with 2-fluoro-3-(triazolo[4,5-b]pyridin-2-ylmethyl)benzoic acid, provided the title compound hydrochloride salt as a white solid. LC-MS: [M+H⁺]=496.2. ¹H NMR (400 MHZ, DMSO-d₆): δ ppm 9.31-9.10 (m, 1H), 9.07-8.90 (m, 1H), 8.85 (br d, J=4.1 Hz, 1H), 8.43 (d, J=9.1 Hz, 1H), 8.23 (d, J=5.4 Hz, 1H), 7.61-7.48 (m, 3H), 7.40 (s, 2H), 7.25-7.14 (m, 1H), 6.87-6.78 (m, 1H), 6.78-6.69 (m, 1H), 5.87-5.73 (m, 2H), 5.01-4.87 (m, 1H), 4.87-4.72 (m, 1H), 3.83-3.79 (m, 1H), 3.66-3.50 (m, 1H), 3.41-3.34 (m, 1H), 3.31-3.20 (m, 1H), 3.00-2.93 (m, 1H), 2.91-2.78 (m, 1H), 2.78-2.70 (m, 3H), 2.46 (br s, 1H), 2.07-1.95 (m, 1H), 1.92-1.73 (m, 3H), 1.46-1.32 (m, 1H).

### Synthesis of (R)-4-((3-aminopyridin-2-yl)amino)-2-fluoro-N-(8-methylisoquinolin-1-yl)-N-(piperidin-3-yl)benzamide (Example 274)

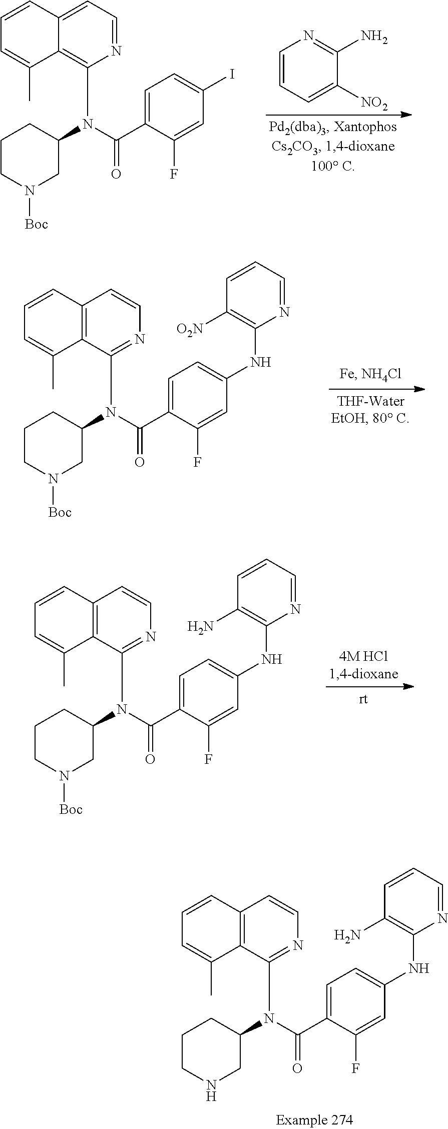 [SMILES: Cc1cccc2ccnc(N(C(=O)c3ccc(I)cc3F)[C@@H]3CCCN(C(=O)OC(C)(C)C)C3)c12 ; Nc1ncccc1[N+](=O)[O-] ; Cc1cccc2ccnc(N(C(=O)c3ccc(Nc4ncccc4[N+](=O)[O-])cc3F)[C@@H]3CCCN(C(=O)OC(C)(C)C)C3)c12 ; Cc1cccc2ccnc(N(C(=O)c3ccc(Nc4ncccc4N)cc3F)[C@@H]3CCCN(C(=O)OC(C)(C)C)C3)c12 ; Cc1cccc2ccnc(N(C(=O)c3ccc(Nc4ncccc4N)cc3F)[C@@H]3CCCNC3)c12]

### Step 1:

To a solution of tert-butyl (R)-3-(4-iodo-N-(8-methylisoquinolin-1-yl)benzamido) piperidine-1-carboxylate (300 mg, 0.52 mmol) in 1,4-dioxane (8 mL) at 25° C. was added Cs₂CO₃ (256 mg, 0.78 mmol), Xanthphos (30 mg, 0.05 mmol) and 3-nitropyridin-2-amine (146 mg, 1.05 mmol) and the reaction mixture was degassed with N₂ gas for 10 min and followed by addition of Pd₂(dba)₃ (24 mg, 0.02 mmol). The reaction mixture was stirred at 100° C. for 4 h before it was diluted with water and extracted with EtOAc (2×40 mL). The organic layer was washed with brine (20 mL), dried over Na₂SO₄, and concentrated. The residue was purified by Combi-flash using 35% EtOAc in hexane to afford tert-butyl (R)-3-(2-fluoro-N-(8-methylisoquinolin-1-yl)-4-((3-nitropyridin-2-yl)amino)benzamido)piperidine-1-carboxylate (206 mg, 65%) as yellow solid. LC-MS: [M+H⁺]=601.3.

### Step 2:

To a solution of tert-butyl (R)-3-(2-fluoro-N-(8-methylisoquinolin-1-yl)-4-((3-nitropyridin-2-yl)amino)benzamido)piperidine-1-carboxylate (200 mg, 0.33 mmol) in THF(H₂O:EtOH (10 mL) were added iron powder (148 mg, 2.66 mmol) followed by NH₄Cl (88 mg, 1.66 mmol) at 25° C. and the mixture was stirred at 80° C. for 3 h. The progress of the reaction was monitored by TLC. The reaction mixture filtered through celite and washed with EtOAc. The organic layers ware washed with brine, dried over Na₂SO₄, and concentrated. The residue was purified by Combi-flash using 80% EtOAc in hexane to afford tert-butyl (R)-3-(4-((3-aminopyridin-2-yl)amino)-2-fluoro-N-(8-methylisoquinolin-1-yl)benzamido)piperidine-1-carboxylate (140 mg, 0.20 mmol, 73%) as light brown solid. LC-MS: [M+H⁺]=571.3.

### Step 3:

To a solution of tert-butyl (R)-3-(4-((3-aminopyridin-2-yl)amino)-2-fluoro-N-(8-methylisoquinolin-1-yl)benzamido)piperidine-1-carboxylate (140 mg, 0.24 mmol) was added 4M HCl in 1,4-dioxane (4 mL) at 0° C. and the reaction mixture was stirred at 25° C. for 2 h. The reaction mixture was concentrated, washed with diethyl ether, and dried by lyophilization for 2 days to afford (R)-4-((3-aminopyridin-2-yl)amino)-2-fluoro-N-(8-methylisoquinolin-1-yl)-N-(piperidin-3-yl)benzamide hydrochloride salt (95 mg, 0.18 mmol, 79%) as yellow solid. LC-MS: [M+H⁺]=471.2.

¹H NMR (CD₃OD, 400 MHZ): δ ppm 8.35 (d, J=5.6 Hz, 1H), 7.68-7.58 (m, 2H), 7.45-7.43 (m, 1H), 7.39-7.34 (m, 2H), 7.25 (d, J=5.6 Hz, 1H), 7.06-7.02 (m, 1H), 6.84-6.80 (m, 1H), 6.69-6.62 (m, 1H), 6.48-6.49 (m, 1H), 4.99-4.92 (m, 1H), 3.88-3.86 (m, 1H), 3.70-3.64 (m, 1H), 3.39-3.27 (m, 2H), 2.92-2.87 (m, 1H), 2.73 (s, 3H), 2.59-2.57 (m, 1H), 1.85-1.75 (m, 2H).

### Synthesis of (R)-2-fluoro-N-(8-methylisoquinolin-1-yl)-N-(piperidin-3-yl)-4-(thiazol-2-ylamino)benzamide (Example 275)

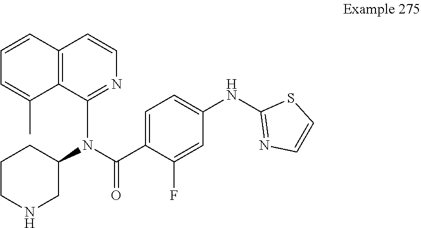 [SMILES: Cc1cccc2ccnc(N(C(=O)c3ccc(Nc4nccs4)cc3F)[C@@H]3CCCNC3)c12]

Example 275 was synthesized by following the procedure for Example 290 by substituting oxazol-2-amine with thiazol-2-amine to obtain the title compound as a hydrochloride salt (40 mg, 91%). LC-MS: [M+H⁺]=462.2. ¹H NMR (CD₃OD, 400 MHZ): δ ppm 8.44-8.40 (m, 1H), 7.76-7.72 (m, 1H), 7.69-7.65 (m, 1H), 7.55-7.46 (m, 2H), 7.35 (d, J=6.0 Hz, 1H), 7.07 (d, J=4.0 Hz, 1H), 7.02-6.99 (m, 1H), 6.95-6.91 (m, 1H), 6.78-6.75 (m, 1H), 5.08-5.02 (m, 1H), 4.00-3.97 (m, 1H), 3.83-3.77 (m, 1H), 3.47-3.34 (m, 1H), 3.01-2.98 (m, 1H), 2.82 (s, 3H), 2.72-2.65 (m, 1H), 1.97-1.90 (m, 2H), 1.58-1.53 (m, 1H).

### Synthesis of (R)-4-((1,7-naphthyridin-8-yl)amino)-2-fluoro-N-(8-methylisoquinolin-1-yl)-N-(piperidin-3-yl)benzamide (Example 276)

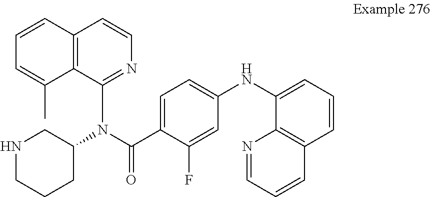 [SMILES: Cc1cccc2ccnc(N(C(=O)c3ccc(Nc4cccc5cccnc45)cc3F)[C@@H]3CCCNC3)c12]

Example 276 was prepared according to the procedure described for Example 185 by substituting pyrimidin-2-amine with 1,7-naphthyridin-8-amine, provided the title compound hydrochloride salt as a white solid. LC-MS (ESI⁺): m/z 507.3 [M+H⁺]. ¹H NMR (400 MHZ, DMSO-d₆) δ ppm 1.27 (td, J=12.27, 7.97 Hz, 1H) 1.58-2.03 (m, 3H) 2.29-2.46 (m, 1H) 2.70-2.87 (m, 3H) 2.89-3.09 (m, 1H) 3.14-3.32 (m, 1H) 3.45-3.81 (m, 1H) 4.79-5.01 (m, 1H) 6.74 (t, J=8.41 Hz, 1H) 7.28 (d, J=6.02 Hz, 1H) 7.35 (br d, J=8.53 Hz, 1H) 7.41-7.63 (m, 2H) 7.69-7.89 (m, 3H) 7.95-8.07 (m, 1H) 8.34 (dd, J=8.22, 1.19 Hz, 1H) 8.42-8.55 (m, 1H) 8.89-8.97 (m, 1H) 9.14-9.30 (m, 1H) 9.46-9.71 (m, 1H) 9.94-10.11 (m, 1H).

### Synthesis of (R)-2-fluoro-4-((1-methyl-1H-pyrazol-3-yl)amino)-N-(8-methylisoquinolin-1-yl)-N-(piperidin-3-yl)benzamide (Example 277)

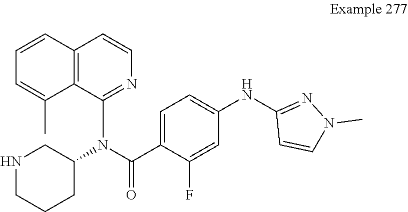 [SMILES: Cc1cccc2ccnc(N(C(=O)c3ccc(Nc4ccn(C)n4)cc3F)[C@@H]3CCCNC3)c12]

Example 277 was prepared according to the procedure described for Example 185 by substituting pyrimidin-2-amine with 1-methyl-1H-pyrazol-3-amine, provided the title compound hydrochloride salt as a white solid. LCMS (ESI⁺): m/z 459.2 [M+H⁺]. ¹H NMR (400 MHZ, DMSO-d₆) δ ppm 1.19-1.38 (m, 1H) 1.64-1.88 (m, 3H) 2.68-2.77 (m, 3H) 2.78-2.93 (m, 1H) 3.19-3.31 (m, 1H) 3.48-3.63 (m, 1H) 3.68 (s, 3H) 4.72-5.00 (m, 2H) 5.63 (br d, J=2.13 Hz, 1H) 6.38-6.56 (m, 2H) 6.88-7.02 (m, 1H) 7.38-7.60 (m, 3H) 7.65-7.87 (m, 2H) 8.38-8.53 (m, 1H) 8.75-8.87 (m, 1H) 8.98-9.11 (m, 1H) 9.17-9.37 (m, 1H).

### Synthesis of (R)—N-(6,8-dimethylisoquinolin-1-yl)-4-(3-methylisoxazol-5-yl)-N-(piperidin-3-yl)piperidine-1-carboxamide (Example 278)

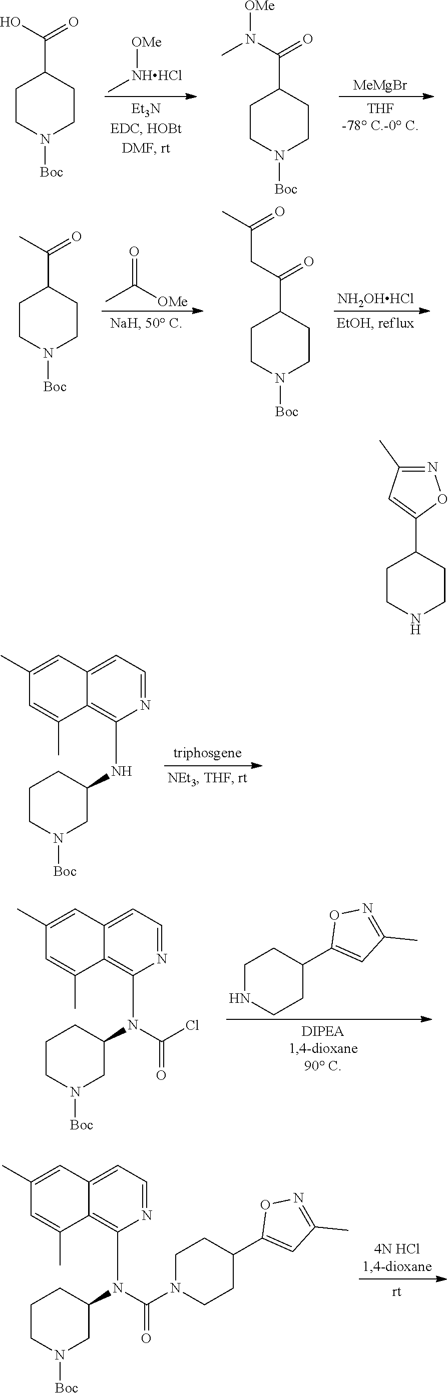

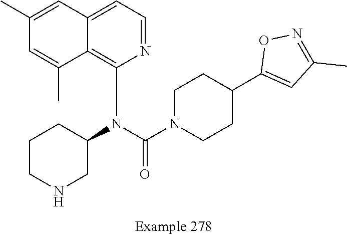

### Step 1:

To a mixture of 1-(tert-butoxycarbonyl)piperidine-4-carboxylic acid (10 g, 43.6 mmol) and N,O-dimethylhydroxylamine hydrochloride (6.3 g, 65.5 mmol) in DMF (100 mL) at 25° C. was added EDC·HCl (10 g, 52.3 mmol) and HOBt (7 g, 52.3 mmol), triethylamine (12 mL, 87.2 mmol). The reaction mixture was stirred for 16 h before it was diluted with 1N aq. HCl (100 mL) and extracted with EtOAc (3×200 mL). The organic layer washed with sat NaHCO₃ solution (100 mL), brine, dried over Na₂SO₄, nd concentrated to afford tert-butyl 4-(methoxy(methyl)carbamoyl)piperidine-1-carboxylate (6 g) as a colorless oil.

### Step 2:

Methyl magnesium bromide in THF (15 mL, 44.11 mmol) at −78° C. was added to a solution of tert-butyl 4-(methoxy(methyl)carbamoyl)piperidine-1-carboxylate (6 g, 22.05 mmol) in THF (60 mL). The reaction mixture was stirred at 0° C. for 2 h before it was quenched with saturated aq. NH₄Cl (40 mL) and extracted with EtOAc (2×60 mL). The organic layer was washed with brine (10 mL), dried over Na₂SO₄, and concentrated. The residue was purified by Combi-flash using 40% EtOAc in hexane to afford tert-butyl 4-acetylpiperidine-1-carboxylate (2.5 g, 11.01 mmol, 50%) as a colorless oil.

### Step 3:

To a solution of tert-butyl 4-acetylpiperidine-1-carboxylate (2 g, 8.81 mmol) in EtOAc (20 mL) at 0° C. was added NaH (635 mg, 44.05 mmol). The resulting mixture was stirred at 50° C. for 16 h before it was quenched with saturated aq. NH₄Cl (30 mL) and extracted with EtOAc (2×50 mL). The organic layer was washed with brine (10 mL), dried over Na₂SO₄, and concentrated to afford tert-butyl 4-(3-oxobutanoyl)piperidine-1-carboxylate (2.1 g, crude) as a colorless oil.

### Step 4:

To a solution of tert-butyl 4-(3-oxobutanoyl)piperidine-1-carboxylate (2.1 g, 7.80 mmol) in Ethanol (20 mL) was added NH₂OH·HCl (1 g, 15.61 mmol) at 25° C. and the reaction mixture stirred at 80° C. for 16 h before it was cooled to 25° C. The reaction mixture was quenched with saturated NH₄Cl (50 mL) and extracted with 10% MeOH in DCM (2×100 mL). The organic layer was washed with brine (10 mL), dried over Na₂SO₄, and concentrated. The residue washed with 10% MeOH in DCM to afford 3-methyl-5-(piperidin-4-yl)isoxazole (800 mg, 66%) as an Off-white solid.

### Step 5:

To a solution of tert-butyl (R)-3-((6,8-dimethylisoquinolin-1-yl)amino)piperidine-1-carboxylate (400 mg, 1.12 mmol) in THF (15 mL) was added NEt₃ (1 mL, 5.05 mmol) at −60° C. ad followed by triphosgene (1 g, 3.37 mmol). The reaction mixture was stirred at 25° C. for 2 h before it was diluted with water (10 mL) and extracted with EtOAc (2×20 mL). The organic layer was washed with brine, dried over Na₂SO₄, and concentrated. The residue was purified by Combi-flash using 50% EtOAc in hexane to afford tert-butyl (R)-3-((chlorocarbonyl)(6,8-dimethylisoquinolin-1-yl)amino)piperidine-1-carboxylate (400 mg, 85%) as a thick oil. LC-MS: [M+H⁺]=418.25.

### Step 6:

To a solution of 5-(1-chloro-115-piperidin-4-yl)-3-methylisoxazole (70 mg, 0.345 mmol) in 1,4-dioxane (2 mL) at 0° C. was added DIPEA (0.22 ml, 1.035 mmol) and followed by tert-butyl (R)-3-((chlorocarbonyl)(6,8-dimethylisoquinolin-1-yl)amino)piperidine-1-carboxylate (115.4 mg, 0.276 mmol) in 1,4-dioxane (2 mL). The reaction mixture was stirred at 90° C. for 6 h. The reaction mixture was diluted with water (10 mL) and extracted with EtOAc (2×10 mL). The organic layer was washed with brine, dried over Na₂SO₄, and concentrated. The residue was purified by Combi-flash using 35% EtOAc in hexane first and then further purified by Prep-HPLC (Conditions: Column: X-SELECT CSH C18 (250*19) mm, 5 μm), Mobile phase A: 0.1% formic acid in H₂O, Mobile phase B: MeCN: MeOH (50:50), Gradient: T/% B: 0/75, 10/85. Flow: 15 mL/min, Dilution: MeCN+H₂O+THF). Pure fractions were dried by lyophilization to afford tert-butyl (R)-3-(N-(6,8-dimethylisoquinolin-1-yl)-4-(3-methylisoxazol-5-yl)piperidine-1-carboxamido)piperidine-1-carboxylate (15 mg, 18%) as an off-white solid. LC-MS: [M+H⁺]=548.3.

### Step 7:

The title compound hydrochloride salt was obtained as an off-white solid by deprotection Method A used in Example 97. LC-MS: [M+H⁺]=448.3. ¹H NMR (CD3OD, 400 MHZ): δ ppm 8.33 (d, J=5.6 Hz, 1H), 7.75 (d, J=5.6 Hz, 1H), 7.68-7.66 (m, 1H), 7.45-7.43 (m, 1H), 5.72 (s, 1H), 4.84-4.76 (m, 1H), 4.92-4.89 (m, 1H), 3.66-3.59 (m, 1H), 3.47 (m, 1H), 3.19-2.89 (m, 3H), 2.70 (s, 3H), 2.50 (s, 3H), 2.32 (m, 1H), 2.17 (s, 3H), 1.96-1.81 (m, 4H), 1.69-1.66 (m, 2H), 1.28-1.24 (m, 2H), 1.07-1.00 (m, 2H).

### Synthesis of ethyl (R)-5-(1-((8-methylisoquinolin-1-yl)(piperidin-3-yl) carbamoyl)piperidin-4-yl)isoxazole-3-carboxylate (Example 279)

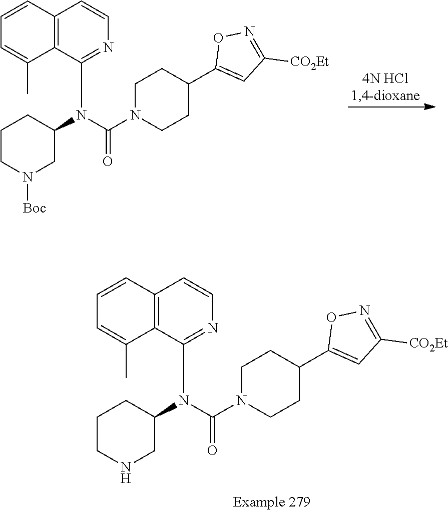 [SMILES: CCOC(=O)c1cc(C2CCN(C(=O)N(c3nccc4cccc(C)c34)[C@@H]3CCCN(C(=O)OC(C)(C)C)C3)CC2)on1 ; "4N HCl1,4-dioxane" ; CCOC(=O)c1cc(C2CCN(C(=O)N(c3nccc4cccc(C)c34)[C@@H]3CCCNC3)CC2)on1]

Example 279 was prepared by removing the Boc protecting group according to the procedure for Example 241. The title compound formic acid salt (30 mg, 37%) was isolated as an off-white solid. LC-MS: [M+H⁺]=492.3. ¹H NMR (CD₃OD, 400 MHZ): δ ppm 8.29 (d, J=5.2 Hz, 1H), 7.76-7.72 (m, 1H), 7.65 (d, J=5.6 Hz, 1H), 7.59-7.52 (m, 1H), 7.42-7.41 (m, 1H), 6.12 (s, 1H), 4.76-4.72 (m, 1H), 4.28 (q, J=7.2 Hz, 2H), 3.78-3.73 (m, 1H), 3.56-3.42 (m, 2H), 2.99-2.96 (m, 1H), 2.79 (m, 2H), 2.61 (s, 3H), 2.44-2.37 (m, 2H), 1.98-1.47 (m, 5H), 1.26 (t, J=7.2 Hz, 3H), 1.20-1.18 (m, 1H), 0.95-0.74 (m, 2H).

### Synthesis of 2-fluoro-N-(8-methyl-1-isoquinolyl)-N-[(3R)-3-piperidyl]-4-(1,2,4-thiadiazol-5-ylamino)benzamide (Example 280)

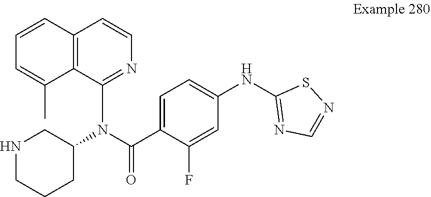 [SMILES: Cc1cccc2ccnc(N(C(=O)c3ccc(Nc4ncns4)cc3F)[C@@H]3CCCNC3)c12]

Example 280 was prepared according to the procedure described for Example 185 by substituting pyrimidin-2-amine with 1,2,4-thiadiazol-5-amine, provided the title compound hydrochloride salt as a white solid. LC-MS: [M+H⁺]=463.2. ¹H NMR (400 MHZ, DMSO-d₆): δ ppm 11.58-11.47 (m, 1H), 9.53-9.30 (m, 1H), 9.24-9.01 (m, 1H), 8.48-8.41 (m, 1H), 8.17 (s, 1H), 7.82-7.78 (m, 1H), 7.72 (br d, J=7.9 Hz, 1H), 7.56-7.50 (m, 1H), 7.49-7.44 (m, 1H), 7.40-7.32 (m, 1H), 6.84-6.81 (m, 1H), 6.76-6.73 (m, 1H), 4.98-4.78 (m, 1H), 3.74 (br d, J=11.5 Hz, 1H), 3.60-3.48 (m, 1H), 3.23 (br d, J=11.5 Hz, 1H), 2.85-2.78 (m, 1H), 2.77-2.73 (m, 3H), 2.00-1.70 (m, 3H), 1.37-1.21 (m, 1H).

### Synthesis of 2-fluoro-N-(8-methyl-1-isoquinolyl)-N-[(3R)-3-piperidyl]-4-[[5-(trifluoromethyl)-1, 3, 4-thiadiazol-2-yl] amino] benzamide (Example #281)

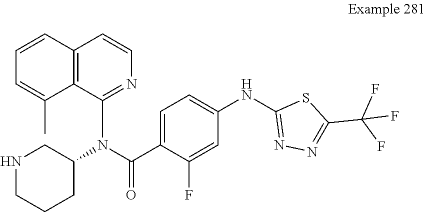 [SMILES: Cc1cccc2ccnc(N(C(=O)c3ccc(Nc4nnc(C(F)(F)F)s4)cc3F)[C@@H]3CCCNC3)c12]

Example 281 was prepared according to the procedure described for Example 185 by substituting pyrimidin-2-amine with 5-(trifluoromethyl)-1,3,4-thiadiazol-2-amine, provided the title compound hydrochloride salt as a white solid. LC-MS [M+H⁺]=531.3. ¹H NMR (400 MHZ, CD3OD): δ ppm 8.52-8.39 (m, 1H), 7.80-7.71 (m, 1H), 7.70-7.63 (m, 1H), 7.57-7.49 (m, 1H), 7.48-7.44 (m, 1H), 7.34 (dd, J=1.8, 12.8 Hz, 1H), 6.87-6.72 (m, 2H), 5.14-5.01 (m, 1H), 4.04-3.93 (m, 1H), 3.89-3.74 (m, 1H), 3.40 (br d, J=12.6 Hz, 1H), 3.11-2.94 (m, 1H), 2.89-2.78 (m, 3H), 2.07-1.83 (m, 3H), 1.69-1.51 (m, 1H).

### Synthesis of (R)-3-((2H-1,2,3-triazol-2-yl)methyl)-2-fluoro-N-(8-methylisoquinolin-1-yl)-N-(piperidin-3-yl)benzamide (Example 282)

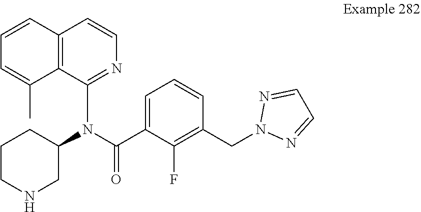 [SMILES: Cc1cccc2ccnc(N(C(=O)c3cccc(Cn4nccn4)c3F)[C@@H]3CCCNC3)c12]

Example 282 was prepared according to the procedure described for Example 271 by substituting 3-((1H-1,2,3-triazol-1-yl)methyl)-2-fluorobenzoic acid with 3-((2H-1,2,3-triazol-2-yl)methyl)-2-fluorobenzoic acid, provided the title compound hydrochloride salt (25 mg, 72%) as an off-white solid. LC-MS: [M+1⁺]=445.2. ¹H NMR (CD₃OD, 400 MHZ): δ ppm 8.24 (d, J=5.2 Hz, 1H), 7.69-7.45 (m, 4H), 7.38-7.26 (m, 2H), 6.86-6.81 (m, 1H), 6.71-6.65 (m, 1H), 6.56-6.52 (m, 1H), 5.30 (s, 2H), 4.96-4.90 (m, 1H), 3.89-3.86 (m, 1H), 3.73-3.67 (m, 1H), 3.30-3.28 (m, 1H), 2.92-2.86 (m, 1H), 2.68 (s, 3H), 1.89-1.80 (m, 3H), 1.49-1.45 (m, 1H).

### Synthesis of (R)-4-(3H-[1,2,3]triazolo[4,5-b]pyridin-3-yl)-N-(7-bromoisoquinolin-1-yl)-2-fluoro-N-(piperidin-3-yl)benzamide (Example 283)

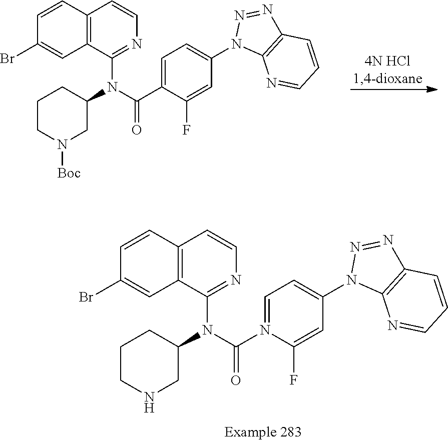

To a solution of tert-butyl (R)-3-(4-(3H-[1,2,3]triazolo[4,5-b]pyridin-3-yl)-N-(7-bromoisoquinolin-1-yl)-2-fluorobenzamido) piperidine-1-carboxylate (90 mg, 0.14 mmol) in DCM (2 mL) at 0° C. was added 4M HCl in 1,4-dioxane (3 mL). The reaction mixture was allowed to warm to room temperature and stirred for 1 h. The reaction mixture was concentrated and dried by lyophilization to R)-4-(3H-[1,2,3]triazolo[4,5-b]pyridin-3-yl)-N-(7-bromoisoquinolin-1-yl)-2-fluoro-N-(piperidin-3-yl)benzamide hydrochloride salt (75 mg, 92%) as an off-white solid. LC-MS: [M+H⁺]=546.1. ¹H NMR (CD₃OD, 400 MHZ): δ ppm 8.76-8.75 (m, 1H), 8.53-8.49 (m, 2H), 8.35-8.31 (m, 1H), 8.09-8.06 (m, 1H), 7.94-7.92 (m, 1H), 7.82-7.75 (m, 3H), 7.54-7.51 (m, 1H), 7.28-7.24 (m, 1H), 5.31-5.21 (m, 1H), 3.97-3.93 (m, 1H), 3.74-3.65 (m, 1H), 2.89-2.86 (m, 1H), 2.51-2.23 (m, 1H), 2.00-1.91 (m, 3H), 1.30-1.28 (m, 1H).

### Synthesis of (R)-4-(3H-[1,2,3]triazolo[4,5-b]pyridin-3-yl)-N-(3-chloro-4-methylpyridin-2-yl)-2-fluoro-N-(piperidin-3-yl)benzamide (Example 284)

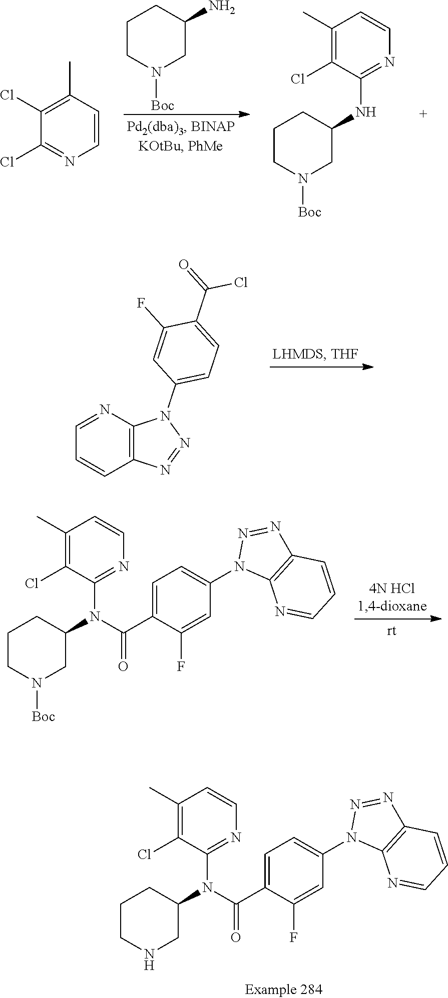

### Step 1:

To a solution of 2,3-dichloro-4-methylpyridine (1 g, 6.172 mmol) & tert-butyl (R)-3-aminopiperidine-1-carboxylate (1.48 g, 7.406 mmol)) in Toluene (30 mL) was added potassium tert butoxide (2.07 g, 18.516 mmol) and Rac BINAP (768 mg, 1.234 mmol) at room temperature. The reaction mixture was degassed with nitrogen for 10 mins. Then Pd₂(dba)₃ (565 mg, 0.617 mmol) was added to the reaction mixture and stirred at 110° C. for 16 h. The reaction mixture was diluted with water (150 ml) and extracted with EtOAc (2×200 mL). The combined organic layer was washed with brine (200 ml), dried over Na₂SO₄, and concentrated. The residue was purified by combi-flash using 20-30% EtOAc in hexane to afford tert-butyl (R)-3-((3-chloro-4-methylpyridin-2-yl) amino)piperidine-1-carboxylate (1.1 g, 55%) as a brown sticky compound. LC-MS: [M+H⁺]=326.25.

### Steps 2-3:

Example 284 was prepared according to the procedure for Example 241 by substituting tert-butyl (R)-3-((8-methylisoquinolin-1-yl)amino)piperidine-1-carboxylate with tert-butyl (R)-3-((3-chloro-4-methylpyridin-2-yl) amino)piperidine-1-carboxylate, provided the title compound trifluoracetic acid salt (20 mg, 35%) as an off-white solid. LC-MS: [M+H⁺]=466.10. ¹H NMR (CD₃OD, 400 MHz): δ ppm 8.82-8.81 (m, 1H), 8.57-8.54 (m, 1H), 8.38-8.36 (m, 1H), 8.25-8.23 (m, 1H), 8.12-8.10 (m, 1H), 7.59-7.57 (m, 1H), 7.50-7.42 (m, 1H), 7.32-7.31 (m, 1H), 5.06-4.95 (m, 1H), 3.83-3.80 (m, 1H), 3.62-3.48 (m, 1H), 3.37-3.24 (m, 1H), 2.92-2.85 (m, 1H), 2.27 (s, 3H), 2.15-1.87 (m, 3H), 1.48-1.42 (m, 1H).

### Synthesis of (R)-2-fluoro-4-(5-isopropyl-2H-tetrazol-2-yl)-N-(8-methylisoquinolin-1-yl)-N-(piperidin-3-yl) benzamide (Example 285)

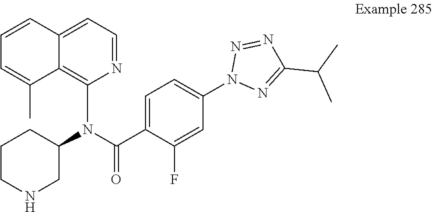 [SMILES: Cc1cccc2ccnc(N(C(=O)c3ccc(-n4nnc(C(C)C)n4)cc3F)[C@@H]3CCCNC3)c12]

Example 285 was prepared according to the procedure for Example 142 by substituting propionimidamide with isobutyrimidamide, provided the title compound formic acid salt as an off-white solid. LC-MS: [M+H⁺]=475.0. ¹H NMR (CD₃OD, 400 MHZ): δ ppm 8.48-8.45 (m, 1H), 7.78-7.74 (m, 1H), 7.70-7.68 (m, 1H), 7.62-7.57 (m, 1H), 7.55-7.49 (m, 3H), 7.13-7.09 (m, 1H), 5.13-5.08 (m, 1H), 4.07-4.04 (m, 1H), 3.87-3.86 (m, 1H), 3.46-3.43 (m, 1H), 3.30-3.26 (m, 1H), 3.08-3.05 (m, 1H), 2.89 (s, 3H), 2.09-1.97 (m, 3H), 1.65-1.63 (m, 1H), 1.40 (d, J=6.8 Hz, 6H).

### Synthesis of (R)-2-fluoro-N-(8-methylisoquinolin-1-yl)-N-(piperidin-3-yl)-4-(5-propyl-2H-tetrazol-2-yl) benzamide) (Example 286)

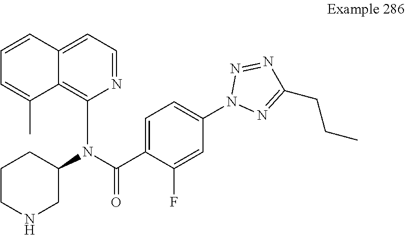 [SMILES: CCCc1nnn(-c2ccc(C(=O)N(c3nccc4cccc(C)c34)[C@@H]3CCCNC3)c(F)c2)n1]

Example 286 was synthesized following the procedure Example 142 by substituting propionimidamide with butyrimidamide, provided the title compound as a hydrochloride salt as an off-white solid. LC-MS: [M+H⁺]=475.0. ¹H NMR (CD₃OD, 400 MHZ): δ ppm 8.50-8.46 (m, 1H), 7.78-7.76 (m, 1H), 7.71-7.69 (m, 1H), 7.63-7.50 (m, 4H), 7.15-7.11 (m, 1H), 5.15-5.09 (m, 1H), 4.08-4.05 (m, 1H), 3.90-3.85 (m, 1H), 3.47-3.44 (m, 1H), 3.09-3.05 (m, 1H), 2.93-2.90 (m, 5H), 2.81-2.77 (m, 1H), 2.01-1.98 (m, 2H), 1.88-1.81 (m, 2H), 1.65-1.63 (m, 1H), 1.04-1.00 (m, 3H).

### Synthesis of (R)—N-(8-methylisoquinolin-1-yl)-4-phenyl-N-(piperidin-3-yl)-3,6-dihydropyridine-1(2H)-carboxamide (Example 287)

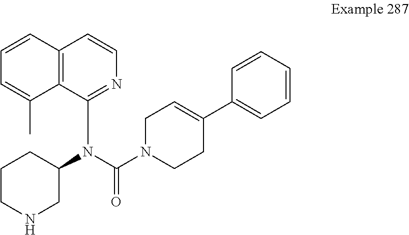 [SMILES: Cc1cccc2ccnc(N(C(=O)N3CC=C(c4ccccc4)CC3)[C@@H]3CCCNC3)c12]

Example 287 was prepared according to the procedure for Example 243 by substituting-methyl-5-(4-piperidyl) isoxazole with 4-phenyl-1,2,3,6-tetrahydropyridine, provided the title compound trifluoroacetic acid salt as a solid. LC-MS: [M+H⁺]=427.3.

### Synthesis of (R)-5-(1-((8-methylisoquinolin-1-yl)(piperidin-3-yl)carbamoyl)piperidin-4-yl)isoxazole-3-carboxamide (Example 288)

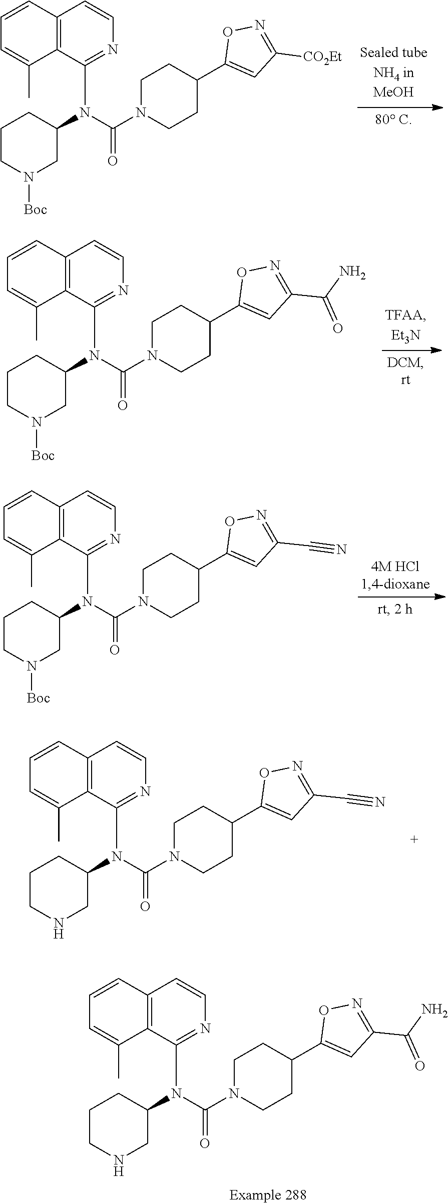

### Step 1:

A mixture of ammonia in MeOH (7M, 5 mL) and ethyl (R)-5-(1-((1-(tert-butoxycarbonyl)piperidin-3-yl)(8-methylisoquinolin-1-yl)carbamoyl)piperidin-4-yl)isoxazole-3-carboxylate (100 mg, 0.16 mmol) in a sealed tube was stirred at 80° C. for 16 h. The reaction mixture was diluted with water (30 mL) and extracted with EtOAc (2×80 mL). The organic layer was washed with brine and concentrated to afford tert-butyl (R)-3-(4-(3-carbamoylisoxazol-5-yl)-N-(8-methylisoquinolin-1-yl)piperidine-1-carboxamido)piperidine-1-carboxylate (70 mg, 73%) as an off-white solid. LC-MS: [M+H⁺]=564.

### Step 2:

To a solution of tert-butyl (R)-3-(4-(3-carbamoylisoxazol-5-yl)-N-(8-methylisoquinolin-1-yl)piperidine-1-carboxamido)piperidine-1-carboxylate (70 mg, 0.12 mmol) in DCM (3 mL) at 25° C. was added triethylamine (0.05 mL, 0.36 mmol) and then trifluoracetic anhydride (0.1 mL, 0.24 mmol). The reaction was stirred at 25° C. for 16 h before it was diluted with water (30 mL) and extracted with DCM (2×30 mL). The combined organic layer was washed with brine and concentrated. The residue was purified by Combi-flash eluting with 100% EtOAc to afford tert-butyl (R)-3-(4-(3-cyanoisoxazol-5-yl)-N-(8-methylisoquinolin-1-yl)piperidine-1-carboxamido)-piperidine-1-carboxylate (45 mg, 67%) as an off-white solid. LC-MS: [M+H⁺]=545.9.

### Step 3:

To a solution of tert-butyl (R)-3-(4-(3-cyanoisoxazol-5-yl)-N-(8-methylisoquinolin-1-yl)piperidine-1-carboxamido)piperidine-1-carboxylate (45 mg, 0.83 mmol) in DCM (1 mL) was added 4N HCl in 1,4-dioxane (1 mL) at 0° C. The reaction mixture was stirred at 25° C. for 2 h and then concentrated. The residue was purified by Prep-HPLC (Conditions: Column: Luna OMEGA C-18 (250×21.2×5 μm), Mobile phase A: 0.1% HCl in water (100%), Mobile phase B: 100% MeCN, Gradient: T/% B: 0/30, 10/55, Flow: 15 mL/min, Dilution: MeCN+H₂O+THF). Pure fractions were dried by lyophilization to afford (R)-4-(3-cyanoisoxazol-5-yl)-N-(8-methylisoquinolin-1-yl)-N-(piperidin-3-yl)piperidine-1-carboxamide as an off-white solid and (R)-5-(1-((8-methylisoquinolin-1-yl)(piperidin-3-yl)carbamoyl)piperidin-4-yl)isoxazole-3-carboxamide formic acid salt (8 mg, 40%) as an off-white solid. LC-MS: [M+H⁺]=463.2. ¹H NMR (CD₃OD, 400 MHZ): δ ppm 8.29 (d, J=5.2 Hz, 1H), 7.76-7.72 (m, 1H), 7.65 (d, J=5.2 Hz, 1H), 7.59-7.53 (m, 1H), 7.42-7.40 (m, 1H), 6.03 (s, 1H), 4.95-4.89 (m, 1H), 4.04 (m, 1H), 3.81-3.43 (m, 3H), 2.96-2.80 (m, 3H), 2.60 (s, 3H), 2.47-2.37 (m, 2H), 2.01-1.51 (m, 5H), 1.20-1.17 (m, 1H), 0.95-0.78 (m, 2H).

### Synthesis of ((R,E)-4-(3H-[1,2,3]triazolo[4,5-b]pyridin-3-yl)-2-fluoro-N-(3-(3-hydroxyprop-1-en-1-yl)pyridin-2-yl)-N-(piperidin-3-yl)benzamide (Example 289)

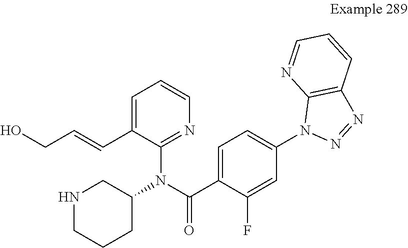 [SMILES: O=C(c1ccc(-n2nnc3cccnc32)cc1F)N(c1ncccc1/C=C/CO)[C@@H]1CCCNC1]

Example 289 was prepared according to the procedure for Example 104 by substituting tert-butyl-dimethyl-[(E)-4-(4,4,5,5-tetramethyl-1,3,2-dioxaborolan-2-yl)but-3-enoxy]silane with (E)-tert-butyldimethyl((3-(4,4,5,5-tetramethyl-1,3,2-dioxaborolan-2-yl)allyl)oxy)silane, provided the title compound as a white solid. LC-MS: [M+H⁺]=474.3. ¹H NMR (400 MHZ, DMSO-d₆): δ ppm 8.96-8.82 (m, 1H), 8.80-8.67 (m, 1H), 8.49-8.33 (m, 1H), 8.03 (dd, J=1.8, 10.6 Hz, 1H), 7.98-7.88 (m, 2H), 7.72-7.58 (m, 1H), 7.48-7.21 (m, 2H), 6.76-6.60 (m, 1H), 6.45-6.30 (m, 1H), 5.14-4.99 (m, 1H), 4.59-4.33 (m, 1H), 4.18 (br d, J=4.4 Hz, 2H), 3.28 (br s, 1H), 3.05-2.76 (m, 2H), 2.31-2.10 (m, 2H), 1.88-1.74 (m, 1H), 1.68-1.37 (m, 2H), 1.16-0.98 (m, 1H).

### Synthesis of ((R)-2-fluoro-N-(8-methylisoquinolin-1-yl)-4-(oxazol-2-ylamino)-N-(piperidin-3-yl) benzamide (Example 290)

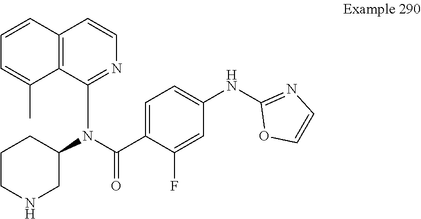 [SMILES: Cc1cccc2ccnc(N(C(=O)c3ccc(Nc4ncco4)cc3F)[C@@H]3CCCNC3)c12]

Example 290 was prepared according to the procedure for Example 119 by substituting 2-aminopyrimidine and tert-butyl (R)-3-(2-fluoro-4-iodo-N-(1-methyl-1H-pyrazolo[3,4-c]pyridin-7-yl)benzamido)piperidine-1-carboxylate with oxazol-2-amine and tert-butyl (R)-3-(2-fluoro-4-iodo-N-(8-methylisoquinolin-1-yl)benzamido)piperidine-1-carboxylate, provided the title compound formic acid salt as a white solid. LC-MS: [M+H⁺]=446.20. ¹H NMR (CD₃OD, 400 MHZ): δ ppm 8.42-8.40 (m, 1H), 7.73-7.72 (m, 1H), 7.70-7.65 (m, 1H), 7.51-7.47 (m, 1H), 7.44-7.39 (m, 2H), 7.17-7.13 (m, 1H), 6.86 (s, 1H), 6.65-6.58 (m, 2H), 5.02-5.00 (m, 1H), 3.91-3.88 (m, 1H), 3.76-3.73 (m, 1H), 2.91-2.82 (m, 2H), 2.79 (s, 3H), 1.93-1.90 (m, 3H), 1.55-1.53 (m, 1H).

### Synthesis of (R)-2-fluoro-N-(isoquinolin-3-yl)-4-(isoquinolin-5-yl)-N-(piperidin-3-yl)benzamide (Example 291)

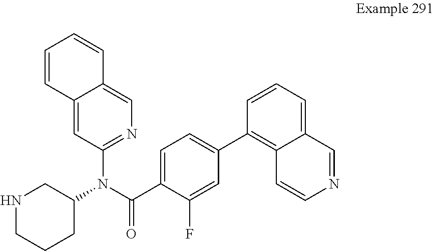

Example 291 was prepared according to the procedure described for Example 241 by substituting 2-fluoro-4-(1-methyltriazol-4-yl)benzoic acid and tert-butyl (3R)-3-[(6-chloro-1-isoquinolyl)amino]piperidine-1-carboxylate with 2-fluoro-4-(isoquinolin-5-yl)benzoic acid and tert-butyl (R)-3-(isoquinolin-3-ylamino)piperidine-1-carboxylate, provided the title compound hydrochloride salt (70%) as a white solid. LC-MS: [M+H⁺]=477.3.

### Synthesis of (R)-2-fluoro-N-(8-methylisoquinolin-1-yl)-4-((6-oxo-1,6-dihydropyridin-2-yl)amino)-N-(piperidin-3-yl)benzamide (Example 292)

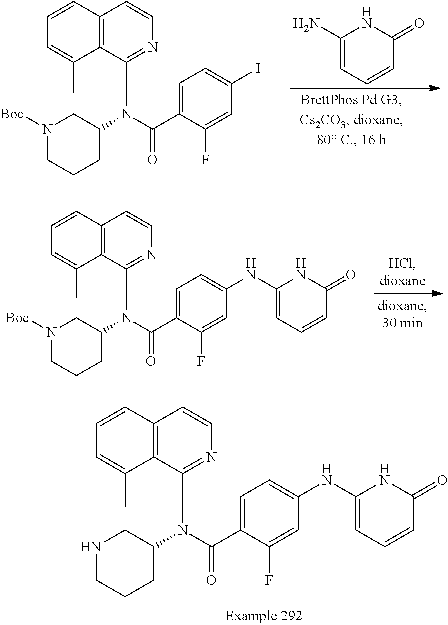

Example 292 was prepared according to the procedure described for Example 131 by substituting 3-amino-1-methyl-pyridin-2-one with 6-aminopyridin-2 (1H)-one, provided the title compound hydrochloride salt as a white solid. LCMS (ESI+): m/z 472.3 [M+H⁺]; ¹H NMR (ET69784-209-P1A2, 400 MHz, DMSO-d₆) δ ppm 1.19-1.36 (m, 1H), 1.65-1.99 (m, 3H), 2.73 (s, 3H), 2.76-2.94 (m, 1H), 3.21 (br d, J=12.01 Hz, 1H), 3.44-3.63 (m, 1H), 3.70 (br d, J=10.88 Hz, 1H), 4.82-5.04 (m, 1H), 6.05 (d, J-7.88 Hz, 1H), 6.23 (d, J=7.88 Hz, 1H), 6.54 (t, J=8.50 Hz, 1H), 6.74 (dd, J=8.63, 1.75 Hz, 1H), 7.37-7.47 (m, 2H), 7.50-7.62 (m, 2H), 7.73 (br d, J=8.00 Hz, 1H), 7.77-7.85 (m, 1H), 8.43-8.49 (m, 1H), 9.23-9.42 (m, 2H), 9.76 (br d, J=10.01 Hz, 1H).

### Synthesis of (R)-3-(3-fluoro-4-((8-methylisoquinolin-1-yl)(piperidin-3-yl)carbamoyl)phenyl)-3H-[1,2,3]triazolo[4,5-b]pyridine-5-carboxylic acid (Example 293)

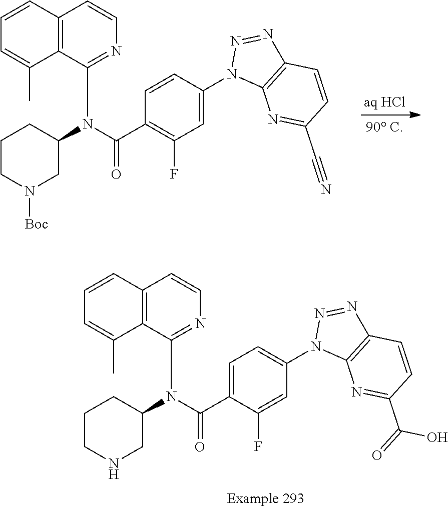 [SMILES: Cc1cccc2ccnc(N(C(=O)c3ccc(-n4nnc5ccc(C#N)nc54)cc3F)[C@@H]3CCCN(C(=O)OC(C)(C)C)C3)c12 ; Cc1cccc2ccnc(N(C(=O)c3ccc(-n4nnc5ccc(C(=O)O)nc54)cc3F)[C@@H]3CCCNC3)c12]

To a solution of tert-butyl (R)-3-(4-(5-cyano-3H-[1,2,3]triazolo[4,5-b]pyridin-3-yl)-2-fluoro-N-(8-methylisoquinolin-1-yl)benzamido)piperidine-1-carboxylate (30 mg, 0.0494 mmol) in water (5 mL) at 0° C. was added 50% HCl (2 mL). The reaction mixture was stirred at 90° C. for 16 h. The reaction mixture was concentrated, and the residue was further purified by Prep-HPLC (Conditions: Column: Kinetex C-18 (250×21.2×5 μm), Mobile phase A: 0.1% formic acid in H₂O (100%), Mobile phase B: 100% MeCN, Gradient: Time (min)/% B: 0/30, 10/60, Flow: 15 mL/min, Dilution: MeCN+H₂O+THF). Pure fractions were combined and dried by lyophilization to afford (R)-3-(3-fluoro-4-((8-methylisoquinolin-1-yl)(piperidin-3-yl)carbamoyl)-phenyl)-3H-[1,2,3]triazolo[4,5-b]pyridine-5-carboxylic acid—formic acid (35 mg, 70%) as an off-white solid. LC-MS: [M+H⁺]=526.95. ¹H NMR (CD₃OD, 400 MHz): δ ppm 8.51-8.42 (m, 2H), 8.14-8.08 (m, 3H), 7.75-7.71 (m, 1H), 7.66-7.61 (m, 1H), 7.48-7.43 (m, 2H), 7.11-7.06 (m, 1H), 5.11-4.98 (m, 1H), 4.05-4.02 (m, 1H), 3.88-3.82 (m, 1H), 3.48-3.40 (m, 1H), 3.08-2.99 (m, 1H), 2.75 (s, 3H), 2.02-1.82 (m, 3H), 1.59-1.57 (m, 1H).

### Synthesis of 2-methyl-4-[4-[(8-methyl-1-isoquinolyl)-[(3R)-3-piperidyl] carbamoyl] phenyl] pyrazole-3-carboxylic acid (Example 294)

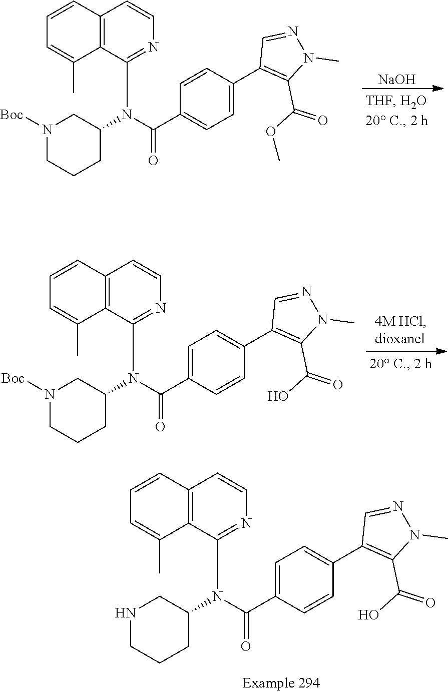

### Step 1:

A mixture of tert-butyl (3R)-3-[[4-(5-methoxycarbonyl-1-methyl-pyrazol-4-yl) benzoyl]-(8-methyl-1-isoquinolyl) amino] piperidine-1-carboxylate (100 mg, 171.33 μmol), NaOH (27.41 mg, 685.31 μmol) in THF (1 mL) and H₂O (1 mL) was stirred at 20° C. for 2 h under N₂ atmosphere. The reaction mixture was filtered and concentrated, which was purified by prep-HPLC (column: Waters Xbridge C18 150*50 mm*10 μm; mobile phase: [H₂O (10 mM NH₄HCO₃)-MeCN]; gradient: 20%-40% B over 8.0 min) to give 4-[4-[[(3R)-1-tert-butoxycarbonyl-3-piperidyl]-(8-methyl-1-isoquinolyl) carbamoyl] phenyl]-2-methyl-pyrazole-3-carboxylic acid (65 mg, 66%) as a white solid.

### Step 2:

The title compound hydrochloride salt was obtained as a white solid by deprotection Method A used in Example 97. LC-MS [M+H⁺]=470.3. ¹H NMR (400 MHZ, CD₃OD) δ ppm 8.49 (d, J=5.5 Hz, 1H), 7.83 (d, J=5.6 Hz, 1H), 7.71 (d, J=8.1 Hz, 1H), 7.50 (t, J=7.7 Hz, 1H), 7.39 (d, J=7.0 Hz, 1H), 7.31 (s, 1H), 7.11-7.00 (m, 4H), 5.15-5.03 (m, 1H), 4.06 (s, 3H), 3.95 (br d, J=11.9 Hz, 1H), 3.87-3.75 (m, 1H), 3.39 (br d, J=12.5 Hz, 1H), 3.06-2.92 (m, 1H), 2.86-2.70 (m, 3H), 2.00-1.88 (m, 2H), 1.83 (br d, J=12.8 Hz, 1H), 1.57-1.40 (m, 1H).

### Synthesis of (R,E)-3-(1-(4-(3H-[1,2,3]triazolo[4,5-b]pyridin-3-yl)-2-fluoro-N-(piperidin-3-yl)benzamido)isoquinolin-7-yl)acrylic acid (Example 295)

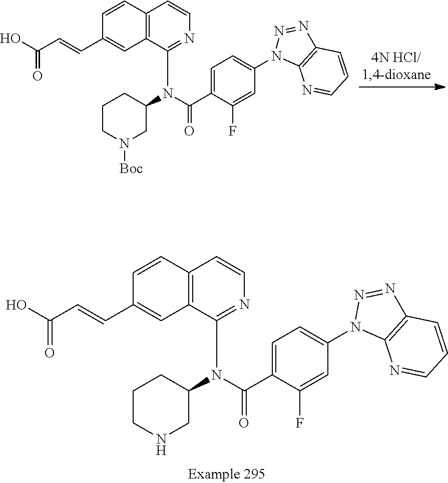 [SMILES: CC(C)(C)OC(=O)N1CCC[C@@H](N(C(=O)c2ccc(-n3nnc4cccnc43)cc2F)c2nccc3ccc(/C=C/C(=O)O)cc23)C1 ; O=C(O)/C=C/c1ccc2ccnc(N(C(=O)c3ccc(-n4nnc5cccnc54)cc3F)[C@@H]3CCCNC3)c2c1]

The title compound was obtained as an off-white solid by deprotection Method A used in Example 97. LC-MS: [M+H⁺]=538.2. ¹H NMR (CD₃OD, 400 MHZ): δ ppm 8.74-8.73 (m, 1H), 8.51-8.48 (m, 2H), 8.29-8.24 (m, 1H), 8.05-7.98 (m, 2H), 7.94-7.84 (m, 3H), 7.78-7.76 (m, 1H), 7.53-7.50 (m, 1H), 7.30-7.25 (m, 1H), 6.72 (d, J=16.6 Hz, 1H), 5.34-5.31 (m, 1H), 3.99-3.97 (m, 1H), 3.76-3.70 (m, 1H), 2.91-2.82 (m, 1H), 2.61-2.40 (m, 1H), 2.05-2.01 (m, 1H), 1.98-1.82 (m, 2H), 1.38-1.28 (m, 1H).

### Synthesis of (R)-2-fluoro-N-(8-methylisoquinolin-1-yl)-N-(piperidin-3-yl)-4-(4,5,6,7-tetrahydro-1H-pyrazolo[4,3-c]pyridin-3-yl)benzamide (Example 296)

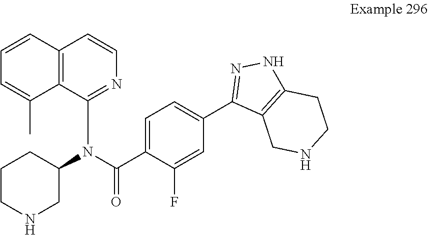 [SMILES: Cc1cccc2ccnc(N(C(=O)c3ccc(-c4n[nH]c5c4CNCC5)cc3F)[C@@H]3CCCNC3)c12]

Example 296 was prepared according to the procedure as described for Example 127 by substituting 2-bromo-5-methyl-1,3,4-thiadiazole with tert-butyl 3-iodo-1,4,6,7-tetrahydro-5H-pyrazolo[4,3-c]pyridine-5-carboxylate, provided the title compound formate salt as an off-white solid. LC-MS: [M+H⁺]=485.3. ¹H NMR (CD₃OD, 400 MHZ): δ ppm 8.46-8.42 (m, 2H), 7.76-7.66 (m, 2H), 7.55-7.50 (m, 2H), 7.07-7.05 (m, 1H), 6.98-6.91 (m, 2H), 5.09-4.83 (m, 1H), 4.20 (s, 2H), 4.00-3.99 (m, 1H), 3.84-3.82 (m, 1H), 3.44-3.42 (m, 3H), 3.01-2.90 (m, 3H), 2.87 (s, 3H), 1.97-1.96 (m, 3H), 1.60-1.52 (m, 1H).

### Synthesis of (R)-4-(2-(benzyloxy)phenyl)-4-hydroxy-N-(8-methylisoquinolin-1-yl)-N-(piperidin-3-yl)piperidine-1-carboxamide (Example #297)

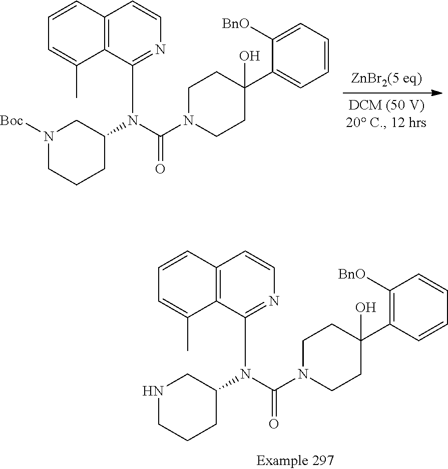 [SMILES: Cc1cccc2ccnc(N(C(=O)N3CCC(O)(c4ccccc4OCc4ccccc4)CC3)[C@@H]3CCCN(C(=O)OC(C)(C)C)C3)c12 ; Cc1cccc2ccnc(N(C(=O)N3CCC(O)(c4ccccc4OCc4ccccc4)CC3)[C@@H]3CCCNC3)c12]

To a solution of tert-butyl (R)-3-(4-(2-(benzyloxy)phenyl)-4-hydroxy-N-(8-methylisoquinolin-1-yl)piperidine-1-carboxamido)piperidine-1-carboxylate (0.05 g, 69.15 μmol) in DCM (2.5 mL) was added ZnBr₂ (77.86 mg, 345.73 μmol), the resulting mixture was stirred at 20° C. for 12 h. The reaction mixture was filtered, and the filtrate was diluted with water (10 mL), then extracted with DCM (3×10 mL). The combined organic layer was washed with brine, dried over Na₂SO₄, then filtered and concentrated. The residue was purified by prep-HPLC (column: Waters Xbridge BEH C18 100*30 mm*10 μm; mobile phase: [H₂O (10 mM NH₄HCO₃)-MeCN]; gradient: 35%-65% B over 8.0 min) and lyophilized to afford (R)-4-(2-(benzyloxy)phenyl)-4-hydroxy-N-(8-methylisoquinolin-1-yl)-N-(piperidin-3-yl)piperidine-1-carboxamide (0.01 g, 23%) as white solid. LC-MS [M+H⁺]=551.3. ¹HNMR (400 MHZ, DMSO-d₆): δ ppm 1.03-1.27 (m, 2H) 1.30-1.67 (m, 3H) 1.80-2.19 (m, 3H) 2.22-2.40 (m, 2H) 2.55-2.70 (m, 4H) 2.72-2.89 (m, 3H) 3.00-3.13 (m, 1H) 3.23 (br t, J=11.01 Hz, 1H) 3.40 (br s, 1H) 3.72-4.30 (m, 1H) 4.96-5.15 (m, 2H) 6.79-6.98 (m, 2H) 7.05-7.16 (m, 1H) 7.29-7.49 (m, 7H) 7.51-7.61 (m, 1H) 7.63-7.68 (m, 1H) 7.70-7.80 (m, 1H) 8.24-8.37 (m, 1H).

### Synthesis of 4-(5-(1-chloroethyl)-2H-tetrazol-2-yl)-2-fluoro-N-(8-methylisoquinolin-1-yl)-N—((R)-piperidin-3-yl) benzamide (Example 298)

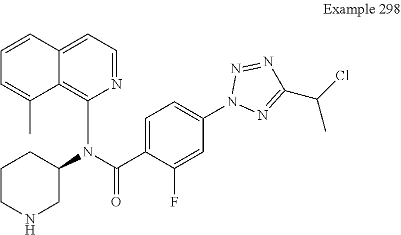 [SMILES: Cc1cccc2ccnc(N(C(=O)c3ccc(-n4nnc(C(C)Cl)n4)cc3F)[C@@H]3CCCNC3)c12]

Example 298 was synthesized following the procedure for Example 285 by substituting isobutyrimidamide hydrochloride with 2-hydroxy propanimidamide hydrochloride, and the alcohol was replaced by chloride during the acid chloride formation step, provided the title compound as a hydrochloride salt (20 mg, 91%). LC-MS: [M+H⁺]=494.20. ¹H NMR (CD₃OD, 400 MHZ): δ ppm 8.45-8.40 (m, 1H), 7.75-7.71 (m, 1H), 7.65-7.62 (m, 2H), 7.51-7.50 (m, 3H), 7.12-7.09 (m, 1H), 5.46-5.41 (m, 1H), 5.08-5.07 (m, 1H), 4.02-3.98 (m, 1H), 3.85-3.82 (m, 1H), 3.48-3.42 (m, 1H), 3.08-2.98 (m, 1H), 2.73 (s, 3H), 2.21-1.93 (m, 5H), 1.68-1.51 (m, 1H), 1.39-1.28 (m, 1H).

### Synthesis of (R)-4-(2-(benzyloxy)phenyl)-N-(8-methylisoquinolin-1-yl)-N-(piperidin-3-yl)-3,6-dihydropyridine-1(2H)-carboxamide (Example 299)

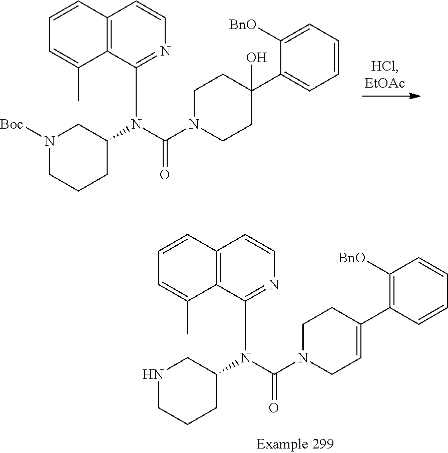 [SMILES: Cc1cccc2ccnc(N(C(=O)N3CCC(O)(c4ccccc4OCc4ccccc4)CC3)[C@@H]3CCCN(C(=O)OC(C)(C)C)C3)c12 ; Cc1cccc2ccnc(N(C(=O)N3CC=C(c4ccccc4OCc4ccccc4)CC3)[C@@H]3CCCNC3)c12]

To a solution of tert-butyl (R)-3-(4-(2-(benzyloxy)phenyl)-4-hydroxy-N-(8-methylisoquinolin-1-yl)piperidine-1-carboxamido)piperidine-1-carboxylate (0.05 g, 69.15 μmol) in EtOAc (1.5 mL) was added 4M HCl in EtOAc (0.5 mL) was stirred at 20° C. for 2 h. The reaction mixture was concentrated. The residue was purified by prep-HPLC (column: Waters Xbridge BEH C18 100*30 mm*10 μm; mobile phase: [H₂O (10 mM NH₄HCO₃)-MeCN]; gradient: 40%-70% B over 8.0 min) and lyophilized to give (R)-4-(2-(benzyloxy)phenyl)-N-(8-methylisoquinolin-1-yl)-N-(piperidin-3-yl)-3,6-dihydropyridine-1(2H)-carboxamide hydrochloride salt (0.01 g, 24%) as white solid. LC-MS [M+H⁺]=533.3. ¹HNMR (400 MHZ, CD₃OD), δ ppm 1.46-1.66 (m, 1H) 1.75-1.98 (m, 4H) 2.13-2.32 (m, 1H) 2.65-2.78 (m, 2H) 2.78-2.96 (m, 4H) 3.12-3.24 (m, 3H) 3.53-3.72 (m, 2H) 4.92-5.01 (m, 2H) 5.31-5.54 (m, 1H) 6.74-7.00 (m, 3H) 7.07-7.18 (m, 1H) 7.25-7.46 (m, 5H) 7.50-7.67 (m, 2H) 7.68-7.86 (m, 2H) 8.14-8.45 (m, 1H).

### Synthesis of (R)-4-(3H-[1,2,3]triazolo[4,5-b]pyridin-3-yl)-2-fluoro-N-(2,7-naphthyridin-1-yl)-N-(piperidin-3-yl)benzamide (Example 300)

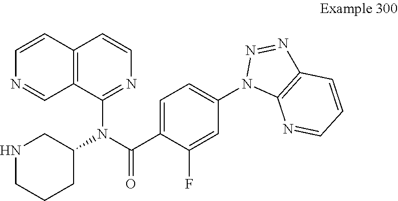 [SMILES: O=C(c1ccc(-n2nnc3cccnc32)cc1F)N(c1nccc2ccncc12)[C@@H]1CCCNC1]

Example 300 was synthesized following the procedure for Example 241 by substituting 6-bromo-1-chloroisoquinoline with 1-chloro-2,7-naphthyridine provided the title compound as a hydrochloride salt. LC-MS: [M+H⁺]=469.65. ¹H NMR (CD₃OD, 400 MHZ) δ ppm 9.82 (bs, 1H), 8.81-8.74 (m, 3H), 8.49 (d, J=6.8 Hz, 1H), 8.05-7.91 (m, 4H), 7.52-7.37 (m, 2H), 5.30 (bs, 1H), 3.98 (bs, 1H), 3.80 (bs, 1H), 3.45-3.33 (m, 1H), 2.88-2.87 (m, 1H), 2.07-1.93 (m, 3H), 1.43 (bs, 1H).

### Synthesis of (R)-1-formyl-N-(8-methylisoquinolin-1-yl)-N-(piperidin-3-yl) piperidine-4-carboxamide (Example 301)

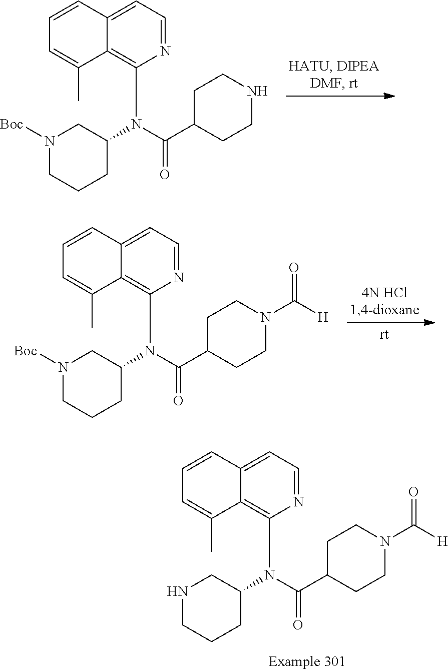

### Step 1:

To a solution of tert-butyl (R)-3-(N-(8-methylisoquinolin-1-yl)piperidine-4-carboxamido)piperidine-1-carboxylate formic acid salt (20 mg, 0.04 mmol) in DMF (2 mL) was added DIPEA (0.02 mL, 0.13 mmol) and HATU (25 mg, 0.06 mmol) at 0° C. and the reaction mixture was stirred at room temperature for 2 h. The reaction mixture was diluted with water (10 mL) and extracted with EtOAc (2×10 mL). The combined organic layer was washed with brine, dried over Na₂SO₄, and concentrated to afford tert-butyl (R)-3-(1-formyl-N-(8-methylisoquinolin-1-yl) piperidine-4-carboxamido) piperidine-1-carboxylate (20 mg, 95%) as an off white solid. LC-MS: [M+H⁺]=481.25.

### Step 2:

The title compound hydrochloride salt was obtained as a white solid by deprotection Method A used in Example 97. LC-MS: [M+H⁺]=381.20. ¹H NMR (CD₃OD, 400 MHZ): δ ppm 8.48 (s, 1H), 7.98-7.94 (m, 2H), 7.88 (d, J=8.8 Hz, 1H), 7.75-7.71 (m, 1H), 7.64-7.62 (m, 1H), 4.77-4.71 (m, 1H), 4.20-3.75 (m, 1H), 3.75-3.47 (m, 2H), 3.40-3.30 (m, 1H), 2.83-2.73 (m, 1H), 2.69 (s, 3H), 2.62-2.47 (m, 1H), 2.45-2.15 (m, 2H), 2.02-2.00 (m, 1H), 1.89-1.64 (m, 5H), 1.48-1.28 (m, 3H), 0.82-0.72 (m, 1H).

### Synthesis of (R)—N-(isoquinolin-1-yl)-N-(piperidin-3-yl)-4-(pyrazolo[1,5-a]pyrimidin-3-yl)benzamide (Example 302)

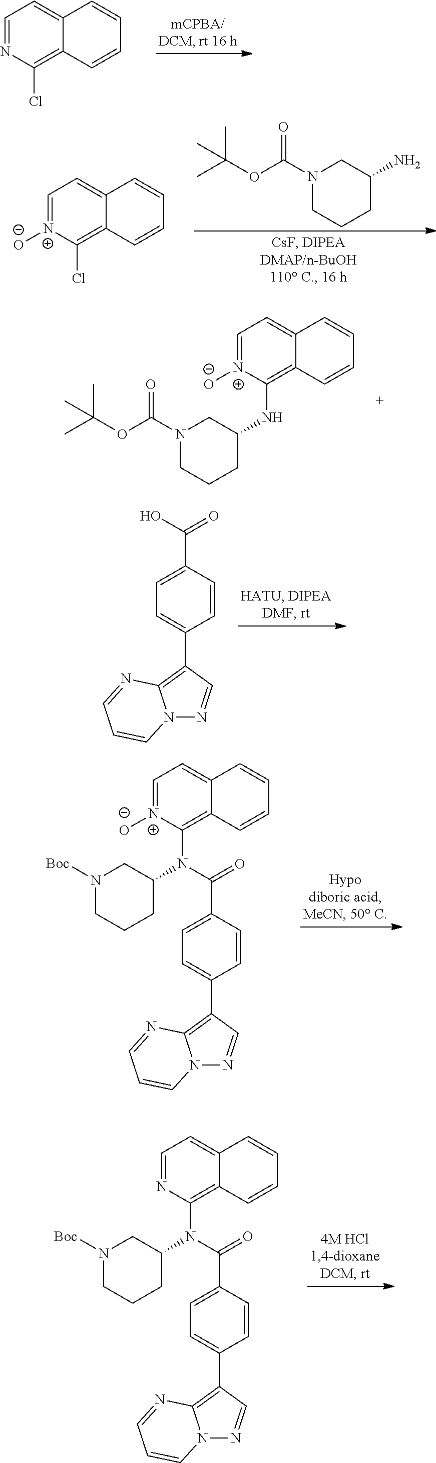

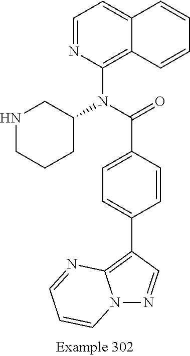 [SMILES: O=C(c1ccc(-c2cnn3cccnc23)cc1)N(c1nccc2ccccc12)[C@@H]1CCCNC1]

### Step 1:

To a stirred solution of 1-chloroisoquinoline (11 g, 67.23 mmol) in DCM (200 mL) at 0° C. was added m-CPBA (34.8 g, 201.7 mmol) portion-wise. The reaction mixture was warmed to room temperature and stirred for 16 h. Aqueous sodium bicarbonate (150 mL) was added to the reaction, and then the mixture was extracted with DCM (2×300 mL). The combined organic layer was dried over Na₂SO₄ and concentrated. The product was purified by Combi-flash, eluting with ethyl acetate to afford 1-chloroisoquinoline 2-oxide (3 g, 24%) as a brown solid. LC-MS: [M+H⁺]=179.60.

### Step 2:

To a stirred solution of 1-chloroisoquinoline 2-oxide (22.25 g, 123.8 mmol) and tert-butyl-(R)-3-aminopiperidine-1-carboxylate (37.21 g, 185.8 mmol) in n-BuOH (150 mL) was added CsF (18.8 g, 123.8 mmol), DMAP (1.5 g, 12.38 mmol), and DIPEA (44 mL, 247.6 mmol). The reaction mixture was stirred at 110° C. for 16 h. The reaction mixture was diluted with water (300 mL), and the mixture was extracted with ethyl acetate (3×500 mL). The combined organic layers were dried over Na₂SO₄ and concentrated. The product was purified by gravity column on silica gel (60-120 mesh) using 4% MeOH in DCM to afford impure (R)-1-((1-(tert-butoxycarbonyl) piperidin-3-yl) amino) isoquinoline 2-oxide (22 g) as a brown solid. LC-MS: [M+H⁺]=344.0.

### Step 3:

To a stirred solution of 4-(pyrazolo[1,5-a]pyrimidin-3-yl)benzoic acid (400 mg, 1.67 mmol) in DMF (10 mL) at 0° C. was added DIPEA (0.88 mL, 5.02 mmol) and HATU (954 mg, 2.51 mmol). The reaction mixture was stirred for 10 min at 0° C., and then (R)-1-((1-(tert-butoxycarbonyl)piperidin-3-yl)amino)isoquinoline 2-oxide (689 mg, 2.01 mmol) was added to the reaction. The resulting mixture was warmed to room temperature and stirred for 16 h. The reaction mixture was diluted with EtOAc (120 mL), washed with cold water (150 mL) and brine (150 mL), dried over Na₂SO₄, and concentrated under reduced pressure. The crude material was purified by flash column chromatography using 4% MeOH in DCM to afford (R)-1-(N-(1-(tert-butoxycarbonyl)piperidin-3-yl)-4-(pyrazolo[1,5-a]pyrimidin-3 yl)benzamido)isoquinoline 2-oxide (200 mg) as a pale yellow solid. LC-MS: [M+H⁺]=565.3.

### Step 4:

To a stirred solution of (R)-1-(N-(1-(tert-butoxycarbonyl)piperidin-3-yl)-4-(pyrazolo[1,5-a]pyrimidin-3-yl)benzamido)isoquinoline 2-oxide (200 mg, 0.35 mmol) in MeCN (10 mL) was added hypodiboric acid (95.2 mg, 1.06 mmol). The reaction mixture was heated to 60° C. and stirred for 1 h. The reaction mixture was cooled to room temperature, diluted with EtOAc (200 mL), and washed with water (200 mL) and brine (200 mL). The organic layer was dried over Na₂SO₄ and concentrated under reduced pressure. The crude material was purified by flash column chromatography using 4% MeOH in DCM to afford the tert-butyl (R)-3-(N-(isoquinolin-1-yl)-4-(pyrazolo[1,5-a]pyrimidin-3-yl)benzamido)piperidine-1-carboxylate (140 mg, 72%) as a yellow solid. LC-MS: [M+H⁺]=549.3

### Step 5:

The title compound hydrochloride salt was obtained as an off-white solid by deprotection Method A used in Example 97. LC-MS: [M+H⁺]=449.3. ¹H NMR (DMSO-d₆, 400 MHZ, 90° C.) δ 9.25 (bs, 1H), 9.00 (d, J=7.2 Hz, 1H), 8.75 (bs, 1H), 8.57 (d, J=4.0 Hz, 1H), 8.53 (s, 1H), 8.46 (d, J=5.6 Hz, 1H), 8.02 (d, J=7.6 Hz, 1H), 7.88 (d, J=8.0 Hz, 1H), 7.81-7.79 (m, 3H), 7.70-7.64 (m, 2H), 7.26-7.24 (m, 2H), 7.04-7.02 (m, 1H), 5.02 (bs, 1H), 3.68-3.67 (m, 1H), 3.24-3.22 (m, 1H), 2.73-2.65 (m, 1H), 2.05-1.81 (m, 4H), 1.61-1.41 (m, 1H).

### Synthesis of (R)—N-(1-methyl-1H-pyrrolo[2,3-c]pyridin-7-yl)-N-(piperidin-3-yl)-4-(pyrazolo[1,5-a]pyrimidin-3-yl)benzamide (Example 303)

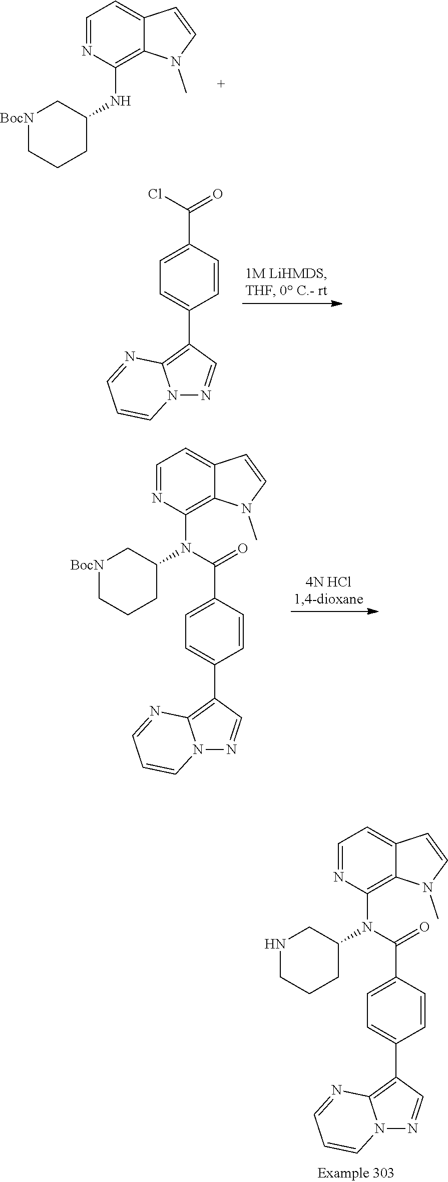

Example 303 was prepared according to the procedure for Example 215 by substituting (R)-3-((8-methylisoquinolin-1-yl)amino)piperidine-1-carboxylate and 2-fluoro-4-(pyrazolo[1,5-a]pyrimidin-3-yl) benzoylchloride with (R)-3-((1-methyl-1H-pyrrolo[2,3-c] pyridin-7-yl) amino)piperidine-1-carboxylate and 4-(pyrazolo[1,5-a]pyrimidin-3-yl) benzoyl chloride, provided the title compound hydrochloride salt as an off-white solid. LC-MS: [M+H⁺]=451.9. ¹H NMR (DMSO-d₆, 400 MHZ) δ 9.15-9.13 (d, J=6.8 Hz, 1H), 8.92-8.84 (bs, 2H), 8.69 (s, 1H), 8.64-8.63 (m, 1H), 8.15-8.09 (m, 1H), 7.90-7.88 (d, J=8.4 Hz, 2H), 7.58-7.52 (m, 1H), 7.47-7.44 (m, 1H), 7.26-7.22 (m, 2H), 7.13-7.10 (m, 1H), 6.45-6.42 (m, 1H), 5.09-5.03 (m, 1H), 3.84 (s, 3H), 3.76-3.73 (m, 1H), 3.56-3.50 (m, 1H), 3.24-3.21 (m, 1H), 2.81-2.67 (m, 1H), 1.77-1.72 (m, 3H), 1.25-1.18 (m, 1H).

### Synthesis of (R,E)-4-(3-(4-(dimethylamino)but-2-enamido)phenyl)-N-(1-methyl-1H-pyrrolo[2,3-c]pyridin-7-yl)-N-(piperidin-3-yl)piperidine-1-carboxamide (Example 304)

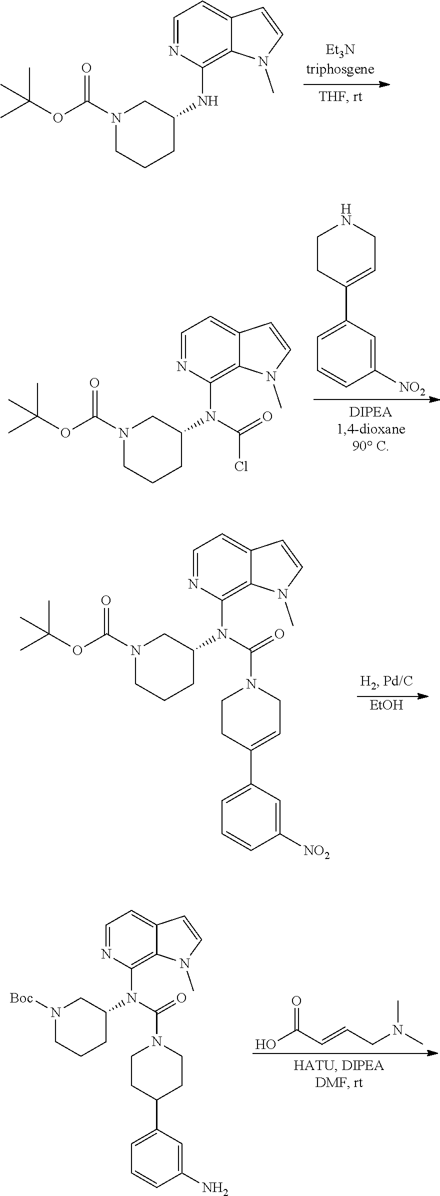

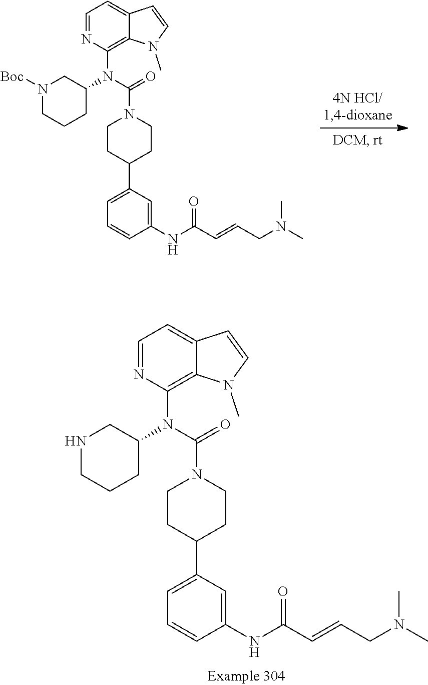 [SMILES: CN(C)C/C=C/C(=O)Nc1cccc(C2CCN(C(=O)N(c3nccc4ccn(C)c34)[C@@H]3CCCN(C(=O)OC(C)(C)C)C3)CC2)c1 ; CN(C)C/C=C/C(=O)Nc1cccc(C2CCN(C(=O)N(c3nccc4ccn(C)c34)[C@@H]3CCCNC3)CC2)c1]

### Step 1:

To a stirred solution of tert-butyl (R)-3-((1-methyl-1H-pyrrolo[2,3-c]pyridin-7-yl)amino)piperidine-1-carboxylate (2.0 g, 6.05 mmol) in THF (50 mL) at −65° C. was added Et₃N (5.0 mL, 36.30 mmol) and triphosgene (5.40 g, 18.15 mmol). The reaction mixture was warmed to room temperature and stirred for 16 h. The reaction mixture was diluted with saturated NH₄Cl (50 mL) and extracted with EtOAc (2×75 mL). The organic layers were dried over Na₂SO₄ and concentrated under reduced pressure. The residue was purified by Combi-flash using 10-20% EtOAc in hexane to afford tert-butyl (R)-3-((chlorocarbonyl)(1-methyl-1H-pyrrolo[2,3-c]pyridin-7-yl)amino)piperidine-1-carboxylate (1.5 g) as a yellow solid. LC-MS: [M+H⁺]=392.9.

### Step 2:

To a stirred solution of 4-(3-nitrophenyl)-1,2,3,6-tetrahydropyridine (1.0 g, 3.30 mmol) in 1,4 dioxane (30 mL) was added DIPEA (4.0 mL, 19.80 mmol) and tert-butyl (R)-3-((chlorocarbonyl)(1-methyl-1H-pyrrolo[2,3-c]pyridin-7-yl)amino)piperidine-1-carboxylate (1.2 g, 3.30 mmol). The reaction mixture was heated to 90° C. and stirred for 16 h. The reaction mixture was diluted with water (80 mL) and extracted with ethyl acetate (2×100 mL). The organic layers were dried over Na₂SO₄, filtered, and concentrated under reduced pressure. The residue was purified by Combi-flash using 20-30% EtOAc in hexane to afford tert-butyl (R)-3-(N-(1-methyl-1H-pyrrolo[2,3-c]pyridin-7-yl)-4-(3-nitrophenyl)-1,2,3,6-tetrahydropyridine-1-carboxamido)piperidine-1-carboxylate (1.0 g) as a yellow solid. LC-MS: [M+H⁺]=561.3.

### Step 3:

To a stirred solution of tert-butyl (R)-3-(N-(1-methyl-1H-pyrrolo[2,3-c]pyridin-7-yl)-4-(3-nitrophenyl)-1,2,3,6-tetrahydropyridine-1-carboxamido)piperidine-1-carboxylate (1.0 g, 1.78 mmol) in EtOH (50 mL) was added 10% Pd/C (700 mg). The reaction mixture was stirred at room temperature for 16 h under hydrogen. The reaction mixture was filtered through a small pad of celite and washed with ethyl acetate (100 mL). The filtrate was concentrated under reduced pressure, and the residue was purified by Combi-flash using 50-70% EtOAc in hexane to afford tert-butyl (R)-3-(4-(3-aminophenyl)-N-(1-methyl-1H-pyrrolo[2,3-c]pyridin-7-yl)piperidine-1-carboxamido)piperidine-1-carboxylate (900 mg) as a brown solid. LC-MS: [M+H⁺]=533.5 Step 4:

To a stirred solution of tert-butyl (R)-3-(4-(3-aminophenyl)-N-(1-methyl-1H-pyrrolo[2,3-c]pyridin-7-yl)piperidine-1-carboxamido)piperidine-1-carboxylate (300 mg, 0.56 mmol) in DMF (20 mL) at 0° C. was added HATU (320 mg, 0.84 mmol), DIPEA (0.5 mL, 2.8 mmol), and (E)-4-(dimethylamino)but-2-enoic acid (190 mg, 1.12 mmol). The reaction mixture was allowed to warm to room temperature and was stirred for 16 h. The reaction mixture was diluted with water (50 mL) and extracted with EtOAc (2×80 mL). The organic layers were dried over Na₂SO₄ and concentrated under reduced pressure. The residue was purified by Combi-flash using 5-7% MeOH in DCM to afford tert-butyl (R,E)-3-(4-(3-(4-(dimethylamino)but-2-enamido)phenyl)-N-(1-methyl-1H-pyrrolo[2,3-c]pyridin-7-yl)piperidine-1-carboxamido)piperidine-1-carboxylate 3 (100 mg, 27%) as a brown solid. LC-MS: [M+H⁺]=644.4.

### Step 5:

The title compound hydrochloride salt was obtained as an off-white solid by deprotection Method A used in Example 97. LC-MS: [M+H⁺]=544.4. ¹H NMR (DMSO-d₆ at 90° C., 400 MHz) δ 10.13 (s, 1H), 9.34 (bs, 1H), 8.94 (bs, 1H), 8.02 (d, J=5.2 Hz, 1H), 7.62 (d, J=2.8 Hz, 1H), 7.53 (d, J=5.2 Hz, 1H), 7.43 (d, J=8.0 Hz, 1H), 7.36 (bs, 1H), 7.14 (t, J=8.0 Hz, 1H), 6.84-6.77 (m, 1H), 6.65-6.63 (m, 1H), 6.60 (d, J=3.2 Hz, 1H), 6.52 (d, J=15.2 Hz, 1H). 4.56 (bs, 1H), 3.90 (s, 3H), 3.88-3.38 (m, 2H), 3.69-3.66 (m, 1H), 3.60-3.56 (m, 2H), 3.42-3.40 (m, 1H), 3.19-3.13 (m, 1H), 2.80-2.72 (m, 2H), 2.27 (s, 6H), 2.59-2.53 (m, 2H), 1.90-1.78 (m, 4H), 1.50-1.47 (m, 1H), 1.30-1.25 (m, 1H), 0.97-0.88 (m, 1H), 0.65-0.59 (m, 1H).

### Synthesis of (R)-4-(3H-[1,2,3]triazolo[4,5-b]pyridin-3-yl)-N-(3-chloropyridin-2-yl)-N-(piperidin-3-yl)benzamide (Example 305)

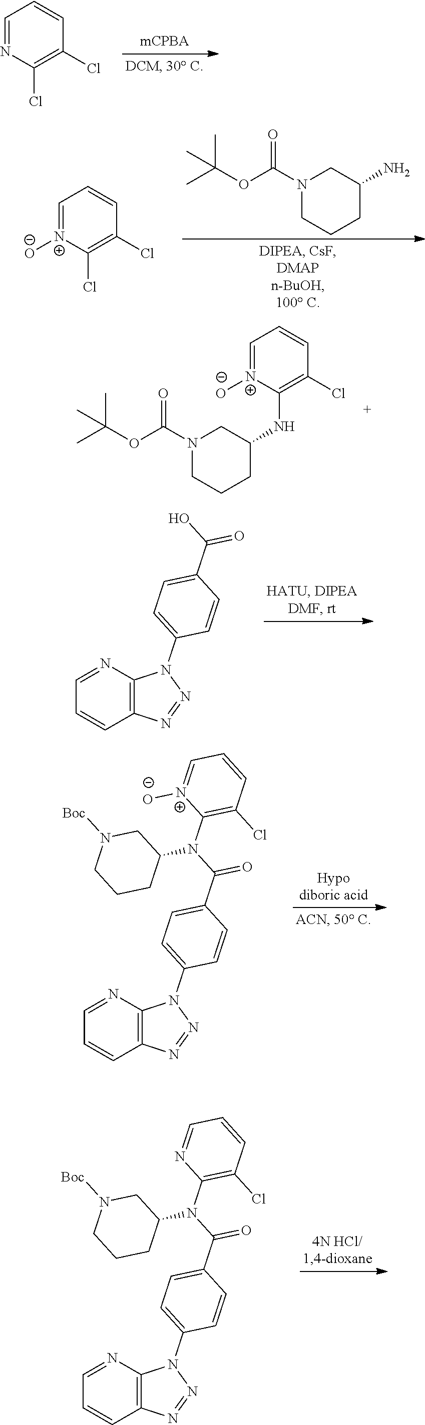

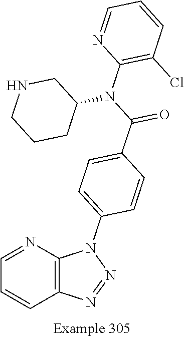 [SMILES: O=C(c1ccc(-n2nnc3cccnc32)cc1)N(c1ncccc1Cl)[C@@H]1CCCNC1]

### Step 1:

To a solution of 2,3-dichloropyridine (60 g, 405 mmol) in DCM at 30° C. was added mCPBA (174.7 g, 1012 mmol). The reaction was stirred for 3 days at 30° C. The reaction mixture was diluted with water (600 mL) and extracted with EtOAc (2×600 mL). The combined organic layers were washed with saturated NaHCO₃ (2×500 mL), brine (500 mL), dried over Na₂SO₄, and concentrated under reduced pressure. The product was purified by flash column chromatography using 5-6% MeOH in DCM to afford 2,3-dichloropyridine 1-oxide (45 g) as a pale brown solid. LC-MS: [M+H⁺]=163.9.

### Step 2:

To a solution of 2,3-dichloropyridine 1-oxide (45.0 g, 274.4 mmol) in n-BuOH at 30° C. were added tert-butyl (R)-3-aminopiperidine-1-carboxylate (82.43 g, 411.6 mmol), CsF (41.68 g, 274.4 mmol), DIPEA (52.57 mL, 301.8 mmol), and DMAP (3.35 g, 27.44 mmol). The reaction was warmed to 110° C. and stirred for 16 h. The solvent was evaporated, and the residue was diluted with water (500 mL). The aqueous phase was extracted with EtOAc (2×750 mL), and the combined organic layers were washed with brine (750 mL), dried over Na₂SO₄, and concentrated. The product was purified by flash column chromatography using 2-4% MeOH in DCM to afford (R)-2-((1-(tert-butoxycarbonyl) piperidin-3-yl) amino)-3-chloropyridine 1-oxide (53 g) as a brown powder. LC-MS: [M+H⁺]=327.9.

### Step 3:

To a stirred solution of (R)-2-((1-(tert-butoxycarbonyl)piperidin-3-yl)amino)-3-chloropyridine 1-oxide (5 g, 15.25 mmol) and 4-(3H-[1,2,3]triazolo[4,5-b]pyridin-3-yl)benzoic acid (4 g, 16.77 mmol) in DMF (50 mL) was added HATU (8.69 g, 22.87 mmol) followed by DIPEA (8.14 ml, 45.75 mmol). The reaction mixture was stirred for 16 h at room temperature. The reaction mixture was diluted with water (200 mL) and extracted with EtOAc (3×200 mL). The combined organic layers were dried over Na₂SO₄ and concentrated. The residue was purified by flash column chromatography (60-120 mesh) using EtOAc to afford (R)-2-(4-(3H-[1,2,3]triazolo[4,5-b]pyridin-3-yl)-N-(1-(tert-butoxycarbonyl)piperidin-3-yl)benzamido)-3-chloropyridine 1-oxide (7 g) as a brownish liquid. LC-MS: [M+H⁺]=550.1.

### Step 4:

To a stirred solution of (R)-2-(4-(3H-[1,2,3]triazolo[4,5-b]pyridin-3-yl)-N-(1-(tert-butoxycarbonyl)piperidin-3-yl)benzamido)-3-chloropyridine 1-oxide (7 g, 12.72 mmol) in MeCN (100 mL) was added hypodiboric acid (2.28 g, 25.44 mmol). The reaction mixture was heated to 50° C. and stirred for 2 h. The reaction mixture was diluted with water (200 mL) and extracted with EtOAc (3×200 mL). The combined organic layers were dried over Na₂SO₄ and concentrated under reduced pressure. The obtained material was purified by flash column chromatography (60-120 mesh) using 50% EtOAc in hexane to afford tert-butyl (R)-3-(4-(3H-[1,2,3]triazolo[4,5-b]pyridin-3-yl)-N-(3-chloropyridin-2-yl)benzamido)piperidine-1-carboxylate (5 g) as a yellow solid. LC-MS: [M-Boc+H⁺]=434.2.

### Step 5:

The title compound hydrochloride salt was obtained as an off-white solid by deprotection Method A used in Example 97. LC-MS: [M+H⁺]=434.3. ¹H NMR (DMSO-d₆ at 90° C., 400 MHz) δ 9.63 (bs, 2H), 8.83-8.82 (m, 1H), 8.64-8.58 (m, 2H), 8.15 (d, J=8.0 Hz, 2H), 7.90 (d, J=8.0 Hz, 1H), 7.60-7.55 (m, 3H), 7.43-7.40 (m, 1H), 4.92 (bs, 1H), 3.58-3.55 (m, 1H), 3.45-3.19 (m, 2H), 2.77-2.70 (m, 1H), 2.05-2.03 (m, 1H), 1.90-1.83 (m, 2H), 1.71-1.63 (m, 1H).

### Synthesis of (R)-4-(3H-[1,2,3]triazolo[4,5-b]pyridin-3-yl)-2-methyl-N-(8-methylisoquinolin-1-yl)-N-(piperidin-3-yl)benzamide (Example 306)

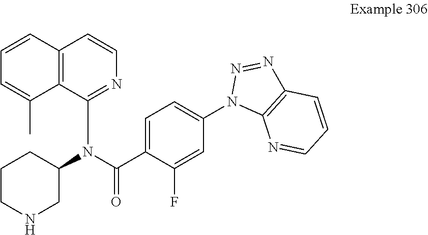 [SMILES: Cc1cccc2ccnc(N(C(=O)c3ccc(-n4nnc5cccnc54)cc3F)[C@@H]3CCCNC3)c12]

Example 306 was prepared following the procedure for Example 241 by substituting 4-(3H-[1,2,3]triazolo[4,5-b]pyridin-3-yl)benzoic acid with 4-(3H-[1,2,3]triazolo[4,5-b]pyridin-3-yl)-2-methylbenzoic acid, provided the title compound hydrochloride salt as a white solid. LC-MS: [M+H⁺]=478.0.

### Synthesis of (R)-4-(3H-[1,2,3]triazolo[4,5-b]pyridin-3-yl)-2-ethyl-N-(8-methylisoquinolin-1-yl)-N-(piperidin-3-yl)benzamide (Example 307)

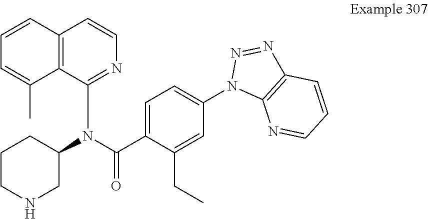 [SMILES: CCc1cc(-n2nnc3cccnc32)ccc1C(=O)N(c1nccc2cccc(C)c12)[C@@H]1CCCNC1]

Example 307 was prepared following the procedure for Example 241 by substituting 4-(3H-[1,2,3]triazolo[4,5-b]pyridin-3-yl)benzoic acid with 4-(3H-[1,2,3]triazolo[4,5-b]pyridin-3-yl)-2-ethylbenzoic, provided the title compound hydrochloride salt as a white solid. LC-MS: [M+H⁺]=492.0.

### Synthesis of (R)-4-(3H-[1,2,3]triazolo[4,5-b]pyridin-3-yl)-2-methoxy-N-(8-methylisoquinolin-1-yl)-N-(piperidin-3-yl)benzamide (Example 308)

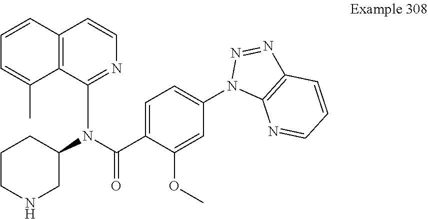 [SMILES: COc1cc(-n2nnc3cccnc32)ccc1C(=O)N(c1nccc2cccc(C)c12)[C@@H]1CCCNC1]

Example 308 was prepared following the procedure for Example 241 by substituting 4-(3H-[1,2,3]triazolo[4,5-b]pyridin-3-yl)benzoic acid with 4-(3H-[1,2,3]triazolo[4,5-b]pyridin-3-yl)-2-methoxybenzoic acid, provided the title compound hydrochloride salt as a white solid. (45 mg) as a white solid. LC-MS: [M+H⁺]=494.6. ¹H NMR (DMSO-d₆, 400 MHz, 90° C.) δ 8.76 (d, J=3.2 Hz, 1H), 8.57 (d, J=8.4 Hz, 1H), 8.35-8.33 (m, 1H), 8.17 (s, 1H), 8.11-7.85 (m, 1H), 7.61-7.40 (m, 8H), 7.02-7.03 (m, 1H), 4.60-4.40 (m, 1H), 4.08-3.80 (m, 1H), 3.66 (s, 3H), 3.29-3.27 (m, 1H), 2.96-2.88 (m, 4H), 1.72-1.60 (m, 3H), 1.28-1.24 (m, 1H).

### Synthesis of (R)—N-(8-methylisoquinolin-1-yl)-N-(piperidin-3-yl)-4-(pyrimidin-2-ylamino)benzamide (Example 309)

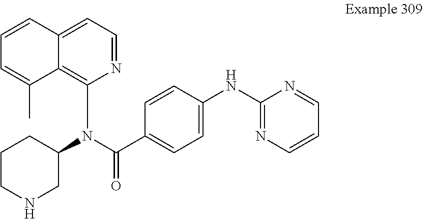 [SMILES: Cc1cccc2ccnc(N(C(=O)c3ccc(Nc4ncccn4)cc3)[C@@H]3CCCNC3)c12]

Example 309 was prepared following the procedure for Example 210 by substituting 2-fluoro-4-nitro benzoyl chloride with 4-nitro benzoyl chloride to afford (R)—N-(8-methylisoquinolin-1-yl)-N-(piperidin-3-yl)-4-(pyrimidin-2-ylamino)benzamide hydrochloride as an off-white solid. LC-MS: [M+H⁺]=439.65. ¹H NMR (CD₃OD, 400 MHz) δ 8.51 (d, J=5.2 Hz, 2H), 8.42 (d, J=5.6 Hz, 1H), 7.75 (d, J=5.6 Hz, 1H), 7.63 (d, J=8.0 Hz, 1H), 7.43-7.29 (m, 2H), 7.18 (d, J=8.8 Hz, 2H), 7.02-7.00 (m, 3H), 5.03-4.96 (m, 1H), 3.86-3.83 (m, 1H), 3.72-3.66 (m, 1H), 3.27-3.20 (m, 1H), 3.03-2.83 (m, 1H), 2.64 (s, 3H), 1.84-1.69 (m, 3H), 1.35-1.30 (m, 1H).

### Synthesis of (R)-4-(3H-[1,2,3]triazolo[4,5-b]pyridin-3-yl)-2-chloro-N-(8-methylisoquinolin-1-yl)-N-(piperidin-3-yl) benzamide (Example 310)

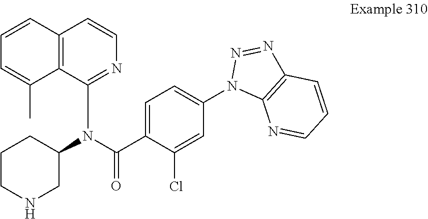 [SMILES: Cc1cccc2ccnc(N(C(=O)c3ccc(-n4nnc5cccnc54)cc3Cl)[C@@H]3CCCNC3)c12]

Example 310 was prepared following the procedure for Example 241 by substituting 4-(3H-[1,2,3]triazolo[4,5-b]pyridin-3-yl)benzoic acid with 4-(3H-[1,2,3]triazolo[4,5-b]pyridin-3-yl)-2-chlorobenzoic acid to afford (R)-4-(3H-[1,2,3]triazolo[4,5-b] pyridin-3-yl)-2-chloro-N-(8-methylisoquinolin-1-yl)-N-(piperidin-3-yl) benzamide hydrochloride salt as an off-white solid. LC-MS: [M+H⁺]=498.55. ¹H NMR (CD₃OD, 400 MHZ) δ 8.69 (d, J=4.4 Hz, 1H), 8.44 (d, J=8.4 Hz, 1H), 8.32 (d, J=5.6 Hz, 1H), 8.22 (s, 1H), 7.82 (dd, J=8.4 Hz, 1.2 Hz, 1H), 7.63-7.45 (m, 5H), 7.10 (d, J=9.2 Hz, 1H), 5.04-4.98 (m, 1H), 4.09-4.06 (m, 1H), 3.76 (t, J=11.4 Hz, 1H), 3.61-3.53 (m, 1H), 3.36-3.33 (m, 1H), 3.06-2.90 (m, 4H), 1.91 (s, 3H), 1.45-1.62 (m, 1H).

### Synthesis of (R)-2-fluoro-N-(8-methylisoquinolin-1-yl)-4-morpholino-N-(piperidin-3-yl)benzamide (Example 311)

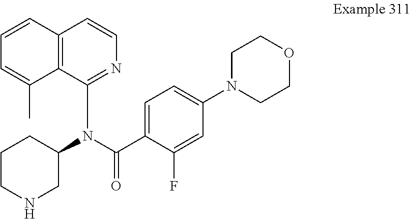 [SMILES: Cc1cccc2ccnc(N(C(=O)c3ccc(N4CCOCC4)cc3F)[C@@H]3CCCNC3)c12]

Example 311 was prepared following the procedure for Example 241 by substituting 4-(3H-[1,2,3]triazolo[4,5-b]pyridin-3-yl)benzoic acid with 2-fluoro-4-morpholinobenzoic acid to afford (R)-2-fluoro-N-(8-methylisoquinolin-1-yl)-4-morpholino-N-(piperidin-3-yl)benzamide. LC-MS: [M+H⁺]=449.0.

### Synthesis of (R)-4-(3H-[1,2,3]triazolo[4,5-b]pyridin-3-yl)-N-(8-methylisoquinolin-1-yl)-N-(piperidin-3-yl)-2-(trifluoromethyl)benzamide (Example 312)

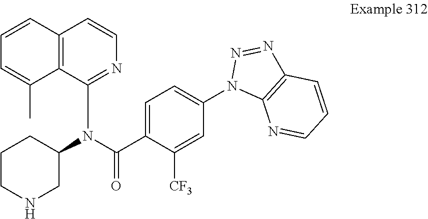 [SMILES: Cc1cccc2ccnc(N(C(=O)c3ccc(-n4nnc5cccnc54)cc3C(F)(F)F)[C@@H]3CCCNC3)c12]

Example 312 was prepared following the procedure for Example 241 by substituting 4-(3H-[1,2,3]triazolo[4,5-b]pyridin-3-yl)benzoic acid with 4-(3H-[1,2,3]triazolo[4,5-b]pyridin-3-yl)-2-(trifluoromethyl)benzoic acid to afford (R)-4-(3H-[1,2,3]triazolo[4,5-b]pyridin-3-yl)-N-(8-methylisoquinolin-1-yl)-N-(piperidin-3-yl)-2-(trifluoromethyl)benzamide hydrochloride salt as an off-white solid. LC-MS: [M+H⁺]=532.65. ¹H NMR (CD₃OD, 400 MHZ) δ 8.67 (dd, J=1.2 Hz, J=4.4 Hz 1H), 8.52 (d, J=6.8 Hz 1H), 8.42 (dd, J=1.2 Hz, J=8.4 Hz 1H), 8.17 (dd, J=3.2 Hz, J=5.6 Hz 1H), 8.07 (dd, J=1.6 Hz, J=8.4 Hz 1H), 7.63-7.55 (m, 4H), 7.46-7.43 (m, 1H), 7.23 (dd, J=4.8 Hz, J=8.8 Hz 1H), 4.08-4.05 (m, 1H), 3.49-3.38 (m, 1H), 3.28-3.24 (m, 1H), 3.05-3.99 (m, 1H), 2.95 (s, 3H), 2.88-2.81 (m, 1H), 1.89-1.78 (m, 3H), 1.25-1.15 (m, 1H).

### Synthesis of (R)-4-(3H-[1,2,3]triazolo[4,5-b]pyridin-3-yl)-2-fluoro-N-(isoquinolin-1-yl)-N-(piperidin-3-yl)benzamide (Example 313)

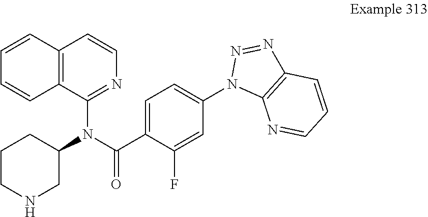 [SMILES: O=C(c1ccc(-n2nnc3cccnc32)cc1F)N(c1nccc2ccccc12)[C@@H]1CCCNC1]

Example 313 was prepared following the procedure for Example 242 by substituting 1-chloro-8-methylisoquinoline for 1-chloroisoquinoline to afford (R)-4-(3H-[1,2,3]triazolo[4,5-b]pyridin-3-yl)-2-fluoro-N-(isoquinolin-1-yl)-N-(piperidin-3-yl)benzamide hydrochloride salt as a white solid. LC-MS: [M+H⁺]=468.5. ¹H NMR (CD₃OD, 400 MHZ) δ 8.75-8.73 (m, 1H), 8.53-8.41 (m, 2H), 8.19-8.17 (m, 1H), 8.03-8.00 (m, 1H), 7.90-7.87 (m, 2H), 7.76-7.72 (m, 3H), 7.53-7.50 (m, 1H), 7.32-7.28 (m, 1H), 5.22-5.18 (m, 1H), 3.87-3.84 (m, 1H), 3.61-3.55 (m, 1H), 3.26-3.22 (m, 1H), 2.79-2.73 (m, 1H), 2.02-1.98 (m, 1H), 1.86-1.81 (m, 2H), 1.28-1.22 (m, 1H).

### Synthesis of (R)-2-fluoro-N-(8-methylisoquinolin-1-yl)-N-(piperidin-3-yl)-4-(thiazol-5-yl) benzamide (Example 314)

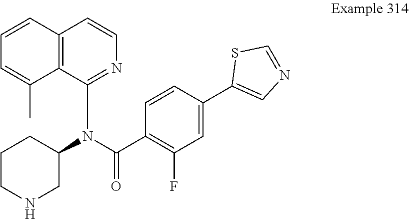 [SMILES: Cc1cccc2ccnc(N(C(=O)c3ccc(-c4cncs4)cc3F)[C@@H]3CCCNC3)c12]

Example 314 was prepared following the procedure for Example 126 by substituting 1-methyl-5-phenyl-1H-pyrazol-4-yl)boronic acid with 5-(4,4,5,5-tetramethyl-1,3,2-dioxaborolan-2-yl) thiazole, the title compound formic acid salt as a white solid. LC-MS: [M+H⁺]=447.35. ¹H NMR (DMSO-d₆, 400 MHz, 90° C.) δ 8.99 (s, 1H), 8.41-8.39 (m, 1H), 8.14-8.11 (m, 1H), 7.73-7.67 (m, 2H), 7.54-7.47 (m, 2H), 7.20 (d, J=8.0 Hz, 1H), 6.99 (d, J=7.6 Hz, 1H), 6.87-6.84 (m, 1H), 4.81-4.52 (m, 1H), 3.75-3.71 (m, 1H), 3.45-3.39 (m, 1H), 2.85-2.83 (m, 1H), 2.79 (s, 3H), 2.68-2.66 (m, 1H), 1.80-1.62 (m, 3H), 1.38-1.24 (m, 1H).

### Synthesis of (R)-2-fluoro-N-(8-methylisoquinolin-1-yl)-4-(4-methylthiazol-2-yl)-N-(piperidin-3-yl)benzamide (Example 315)

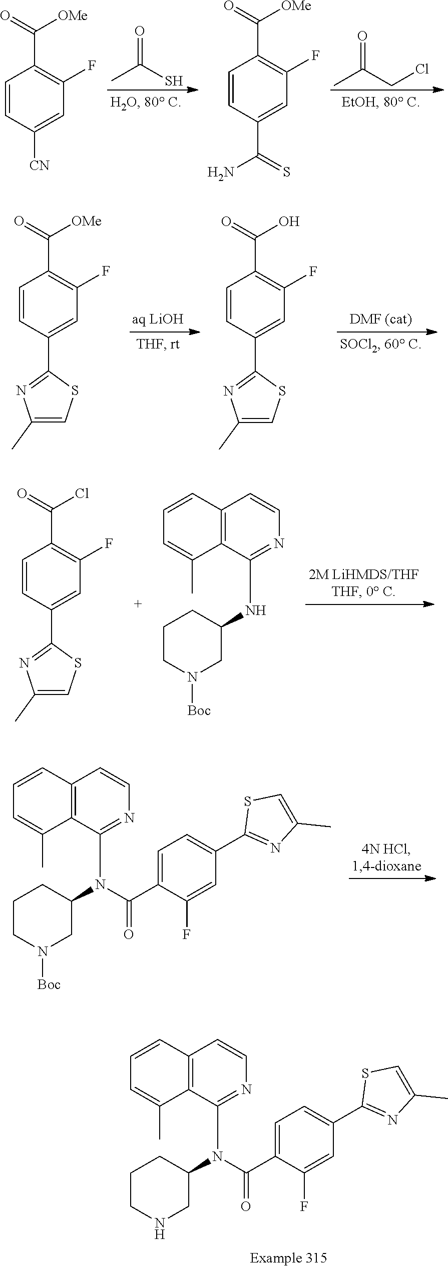

### Step 1:

Ethanethioic S-acid (5 g, 65.69 mmol) and methyl 4-cyano-2-fluorobenzoate (2.3 g, 13.13 mmol) were dissolved in CH₃COOH (6 mL), and the reaction mixture was heated and stirred at 80° C. for 16 h. The reaction mixture was cooled and diluted with water (5 mL) and isopropyl alcohol (5 mL). The precipitated solid was filtered, washed with water, and dried to afford methyl 4-carbamothioyl-2-fluorobenzoate (1.1 g, 8%) as a yellow solid. LC-MS: [M+H⁺]=214.0.

### Step 2:

To a stirred solution of methyl 4-carbamothioyl-2-fluorobenzoate (1.1 g, 5.16 mmol) in EtOH (10 mL) was added 1-chloropropan-2-one (1.2 ml, 15.49 mmol). The reaction was heated to 80° C. and stirred for 4 h. The reaction mixture was cooled to room temperature and concentrated. The precipitate was washed with pentane and dried to afford methyl 2-fluoro-4-(4-methylthiazol-2-yl)benzoate (1.2 g, 4.77 mmol) as a light brown solid. LC-MS: [M+H⁺]=252.25

### Step 3:

To a stirred solution of methyl 2-fluoro-4-(4-methylthiazol-2-yl)benzoate (1.2 g, 4.78 mmol) in THF (5 mL) and H₂O (5 mL) was added LiOH·H₂O (600 mg, 14.34 mmol) at 0° C. The reaction mixture was stirred at 25° C. for 2 h. The reaction mixture was then diluted with water (20 mL) and acidified to pH˜5 with 1N HCl, and a solid precipitated out. The isolated solid was filtered, washed with water, and dried to afford 2-fluoro-4-(4-methylthiazol-2-yl)benzoic acid (900 mg, 79%) as an off-white solid. LC-MS: [M+H⁺]=238.2.

### Step 4:

To a stirred solution of 2-fluoro-4-(4-methylthiazol-2-yl)benzoic acid (100 mg, 0.42 mmol) in SOCl₂ (4 mL) at room temperature was added DMF (0.5 mL), and the reaction mixture was heated and stirred at 60° C. for 16 h. The reaction mixture was cooled to room temperature, concentrated, and co-distilled with toluene to afford 2-fluoro-4-(4-methylthiazol-2-yl)benzoyl chloride (100 mg) as a brown solid, which was used directly in the next step.

### Step 5:

To a stirred solution of (R)—N-(1-(tert-butoxycarbonyl)piperidin-3-yl)-8-methylisoquinolin-1-aminium (500 mg, 1.46 mmol) in THF (6 mL) at 0° C. was added 1 M LiHMDS in THF (4.4 mL, 4.39 mmol), and the reaction was stirred for 40 minutes. Then, 2-fluoro-4-(4-methylthiazol-2-yl)benzoyl chloride (448 mg, 1.75 mmol) in THF (6 mL) was added to reaction mixture at 0° C. and, the reaction was stirred for 5 minutes. The reaction was quenched with saturated NH₄Cl (15 mL) and extracted with ethyl acetate (2×20 mL). The combined organic layers were washed with brine (20 mL), dried over Na₂SO₄, and concentrated. The residue was purified by Combi-Flash using 50% EtOAc in hexane and was further purified by Prep HPLC (Conditions: Column: ZORBAX BONUS-RP (150×21.2×5 μm), Mobile phase A: 10 mM AMMONIUM ACETATE IN H2O, Mobile phase B: 100% MeCN, Gradient: Time (min)/% B: 0/60, 10/90, Flow: 15 mL/min, Dilution: MeCN+H₂O). Pure fractions were combined and dried to afford tert-butyl (R)-3-(2-fluoro-N-(8-methylisoquinolin-1-yl)-4-(4-methylthiazol-2-yl)benzamido)piperidine-1-carboxylate. LC-MS: [M+H⁺]=561.3.

### Step 6:

The title compound hydrochloride salt was obtained as an off-white solid by deprotection Method A used in Example 97. LC-MS: [M+H⁺]=461.30. ¹H NMR (CD₃OD, 400 MHZ) δ 8.34-8.30 (m, 1H), 7.64-7.60 (m, 1H), 7.56-7.51 (m, 1H), 7.43-7.38 (m, 2H), 7.28-7.23 (m, 1H), 7.13-7.11 (m, 2H), 6.85-6.80 (m, 1H), 4.96-4.86 (m, 1H), 3.92-3.69 (m, 1H), 3.75-3.49 (m, 1H), 3.32-3.24 (m, 1H), 2.94-2.90 (m, 1H), 2.74 (s, 3H), 2.30 (s, 3H), 2.11-1.82 (m, 3H), 1.51-1.45 (m, 1H).

### Synthesis of (R)-2-fluoro-N-(8-methylisoquinolin-1-yl)-4-(3-methylisoxazol-5-yl)-N-(piperidin-3-yl) benzamide (Example 316)

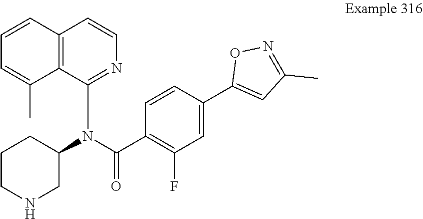 [SMILES: Cc1cc(-c2ccc(C(=O)N(c3nccc4cccc(C)c34)[C@@H]3CCCNC3)c(F)c2)on1]

Example 316 was prepared following the procedure for Example 126 by substituting (1-methyl-5-phenyl-1H-pyrazol-4-yl)boronic acid with 3-methyl-5-(4,4,5,5-tetramethyl-1,3,2-dioxaborolan-2-yl)isoxazole, provided the title compound hydrochloride salt as an off-white solid. LC-MS: [M+H⁺]=445.70. ¹H NMR (CD₃OD, 400 MHZ) δ 8.32 (d, J=7.2 Hz, 1H), 7.64-7.51 (m, 2H), 7.43-7.37 (m, 2H), 7.14-7.10 (m, 1H), 7.01 (dd, J=1.2 Hz, J=8.0 Hz, 1H), 6.85-6.80 (m, 1H), 6.46 (s, 1H), 4.98-4.88 (m, 1H), 3.92-3.72 (m, 1H), 3.38-3.28 (m, 1H), 2.93-2.90 (m, 1H), 2.73 (s, 3H), 2.62-2.61 (m, 1H), 2.14 (s, 3H), 1.89-1.81 (m, 3H), 1.51-1.48 (m, 1H).

### Synthesis of (R)-4-(3H-[1,2,3]triazolo[4,5-b]pyridin-3-yl)-2-fluoro-N-(1-methyl-1H-pyrrolo[2,3-c]pyridin-7-yl)-N-(piperidin-3-yl)benzamide (Example 317)

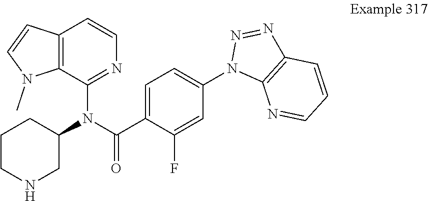 [SMILES: Cn1ccc2ccnc(N(C(=O)c3ccc(-n4nnc5cccnc54)cc3F)[C@@H]3CCCNC3)c21]

Example 317 was made following the procedure for Example 242 by substituting 1-chloro-8-methylisoquinoline with 7-chloro-1-methyl-1H-pyrrolo[2,3-c], provided the title compound hydrochloride salt as an off-white solid. LC-MS: [M+H⁺]=471.70. ¹H NMR (DMSO-d₆, 400 MHz) δ 9.03-9.01 (m, 1H), 8.84-8.80 (m, 1H), 8.71-8.69 (m, 1H), 8.08-8.02 (m, 2H), 7.87-7.85 (m, 1H), 7.63-7.51 (m, 3H), 7.52-7.51 (m, 1H), 7.39-7.33 (m, 1H), 6.49-6.46 (m, 1H), 4.99-4.91 (m, 1H), 4.00 (s, 3H), 3.81-3.78 (m, 1H), 3.24-3.21 (m, 1H), 1.86-1.78 (m, 4H), 1.05-1.02 (m, 1H).

### Synthesis of (R)-6-(3-fluoro-4-((8-methylisoquinolin-1-yl)(piperidin-3-yl)carbamoyl) phenyl) pyrazine-2-carboxamide (Example 318)

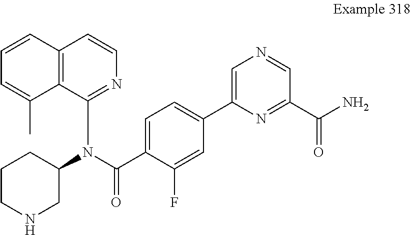 [SMILES: Cc1cccc2ccnc(N(C(=O)c3ccc(-c4cncc(C(N)=O)n4)cc3F)[C@@H]3CCCNC3)c12]

Example 318 was made following the procedure for Example 127 by substituting 2-bromo-5-methyl-1,3,4-thiadiazole with 6-bromopyrazine-2-carbonitrile, provided the title compound hydrochloride salt as an off-white solid. LC-MS: [M+H⁺]=485.3. ¹H NMR (CD₃OD, 400 MHZ) δ 9.02 (s, 2H), 8.32-8.28 (m, 1H), 7.74-7.71 (m, 1H), 7.68-7.49 (m, 3H), 7.40-7.36 (m, 2H), 6.91-6.87 (m, 1H), 5.03-4.94 (m, 1H), 3.92-3.89 (m, 1H), 3.73-3.67 (m, 1H), 3.32-3.27 (m, 1H), 3.01-2.88 (m, 1H), 2.75 (s, 3H), 2.64-2.60 (m, 1H), 1.89-1.83 (m, 2H), 1.26-1.16 (m, 1H).

### Synthesis of (R)-2-fluoro-N-(8-methylisoquinolin-1-yl)-4-((4-methylpyrimidin-2-yl)amino)-N-(piperidin-3-yl)benzamide (Example 319)

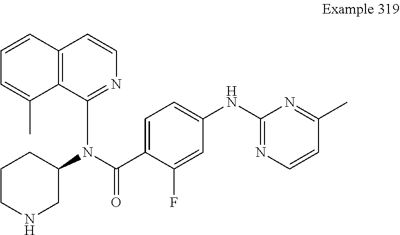 [SMILES: Cc1ccnc(Nc2ccc(C(=O)N(c3nccc4cccc(C)c34)[C@@H]3CCCNC3)c(F)c2)n1]

Example 319 was synthesized following the procedure for Example 131 by substituting 3-amino-1-methyl-pyridin-2-one with 4-methylpyrimidin-2-amine, provided the title compound hydrochloride salt as a white solid. LC-MS: [M+H⁺]=471.3. ¹H NMR (CD₃OD, 400 MHZ) δ 8.46-8.39 (m, 1H), 8.34 (d, J=5.8 Hz, 1H), 7.77-7.71 (m, 1H), 7.70-7.63 (m, 1H), 7.55-7.43 (m, 2H), 7.42-7.32 (m, 1H), 7.00 (d, J=5.8 Hz, 1H), 6.90-6.85 (m, 1H), 6.83-6.76 (m, 1H), 5.12-4.99 (m, 1H), 4.03-3.92 (m, 1H), 3.89-3.76 (m, 1H), 3.48-3.37 (m, 1H), 3.08 (m, 1H), 2.82 (s, 3H), 2.75-2.7 (m 1H), 2.51 (s, 3H), 1.96-1.88 (m, 2H), 1.57-1.50 (m, 1H).

### Synthesis of N-[8-[(E)-3-amino-3-oxo-prop-1-enyl]-1-isoquinolyl]-2-fluoro-N-[(3R)-3-piperidyl]-4-(triazolo[4.5-b]pyridin-3-yl)benzamide (Example 320)

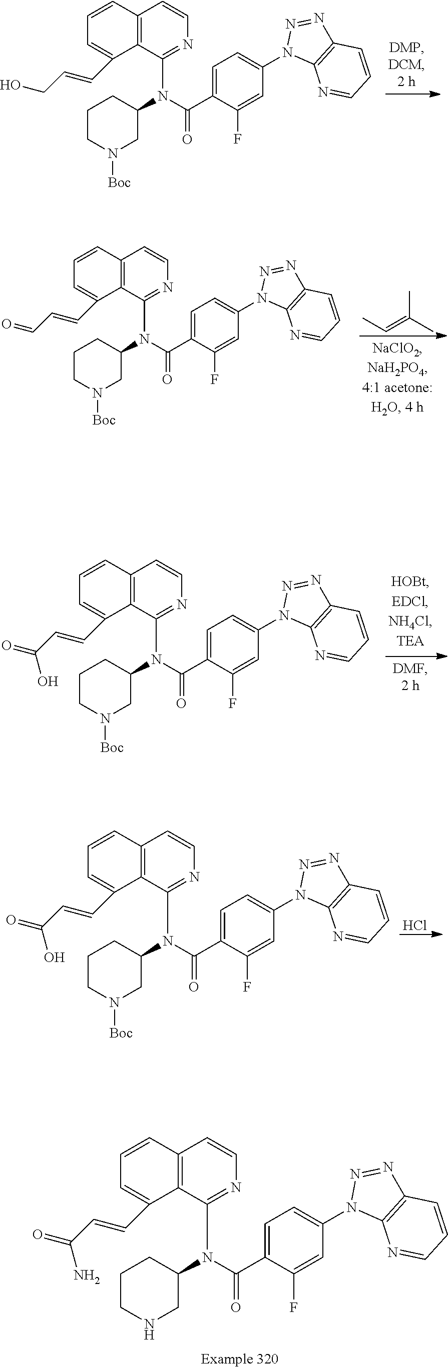

### Step 1:

To a solution of tert-butyl (3R)-3-[[2-fluoro-4-(triazolo[4,5-b]pyridin-3-yl)benzoyl]-[8-[(E)-3-hydroxyprop-1-enyl]-1-isoquinolyl]amino]piperidine-1-carboxylate (420 mg, 606.08 μmol) in DCM (2 mL) was added DMP (334.18 mg, 787.91 μmol). The mixture was stirred at room temperature for 2 h. The reaction mixture was quenched with water (5 mL) and diluted with dichloromethane (5 mL), and then extracted with dichloromethane (3×5 mL). The combined organic layers were washed with brine (3×5 mL), dried over Na₂SO₄, filtered, and concentrated to give tert-butyl (3R)-3-[[2-fluoro-4-(triazolo[4,5-b]pyridin-3-yl)benzoyl]-[8-[(E)-3-oxoprop-1-enyl]-1-isoquinolyl]amino]piperidine-1-carboxylate (350 mg, 83%) as a yellow oil.

### Step 2:

To a solution of tert-butyl (3R)-3-[[2-fluoro-4-(triazolo[4,5-b]pyridin-3-yl)benzoyl]-[8-[(E)-3-oxoprop-1-enyl]-1-isoquinolyl]amino]piperidine-1-carboxylate (350 mg, 563.01 μmol) and 2-methylbut-2-ene (39.49 mg, 563.01 μmol, 59.65 μL) in acetone (4 mL) was added a solution of NaClO₂ (458.28 mg, 5.07 mmol) and NaH₂PO₄ (567.40 mg, 4.73 mmol) in H₂O (1 mL). The mixture was stirred at room temperature for 4 h. The reaction mixture was quenched with water (5 mL) at 0° C., and then diluted with ethyl acetate (20 mL) and extracted with ethyl acetate (3×15 mL). The combined organic layers were washed with brine (3×5 mL), dried over Na₂SO₄, filtered, and concentrated under reduced pressure to give (E)-3-[1-[[(3R)-1-tert-butoxycarbonyl-3-piperidyl]-[2-fluoro-4-(triazolo[4,5-b]pyridin-3-yl)benzoyl]amino]-8-isoquinolyl]prop-2-enoic acid (320 mg, 80%) as a white solid.

### Step 3:

To a solution of (E)-3-[1-[[(3R)-1-tert-butoxycarbonyl-3-piperidyl]-[2-fluoro-4-(triazolo[4,5-b]pyridin-3-yl)benzoyl]amino]-8-isoquinolyl]prop-2-enoic acid (300 mg, 423.42 μmol) in DMF (1 mL) was added TEA (856.92 mg, 8.47 mmol, 1.18 mL), NH₄Cl (226.49 mg, 4.23 mmol), HOBt (137.31 mg, 1.02 mmol), and EDCI (324.68 mg, 1.69 mmol). The mixture was stirred at 25° C. for 12 h. The reaction mixture was quenched with water (5 mL), and then diluted with EtOAc (20 mL) and extracted with ethyl acetate (3×15 mL). The combined organic layer was washed with brine (3×5 mL), dried over Na₂SO₄, filtered and concentrated to give tert-butyl (3R)-3-[[8-[(E)-3-amino-3-oxo-prop-1-enyl]-1-isoquinolyl]-[2-fluoro-4-(triazolo[4,5-b]pyridin-3-yl)benzoyl]amino]piperidine-1-carboxylate (200 mg, 44%) as an off-white solid.

### Step 4:

The title compound hydrochloride salt was obtained as an off-white solid by deprotection Method A used in Example 97. LC-MS: [M+H⁺]=537.4. ¹H NMR (400 MHZ, DMSO-d₆): δ 9.35-9.19 (m, 1H), 9.07-8.91 (m, 1H), 8.84-8.79 (m, 1H), 8.71-8.64 (m, 1H), 8.53-8.43 (m, 1H), 8.23-8.09 (m, 1H), 7.98-7.65 (m, 6H), 7.61 (dd, J=4.5, 8.4 Hz, 1H), 7.46-7.11 (m, 2H), 6.70-6.54 (m, 1H), 4.95-4.33 (m, 1H), 3.89 (br d, J=11.1 Hz, 1H), 3.30-3.13 (m, 1H), 2.90-2.77 (m, 1H), 2.67-2.52 (m, 1H), 2.02-1.60 (m, 3H), 1.54-1.40 (m, 1H).

### Synthesis of (R)-2-fluoro-4-((4-(hydroxymethyl)pyrimidin-2-yl)amino)-N-(8-methylisoquinolin-1-yl)-N-(piperidin-3-yl)benzamide (Example 321)

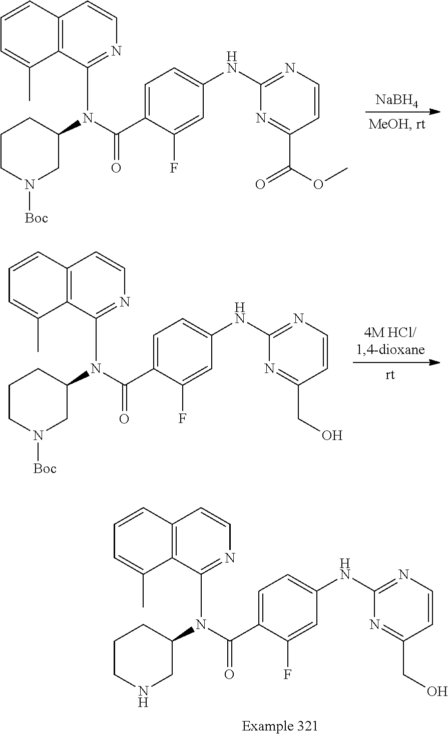

Example 321 was prepared following the procedure for Example 131 by substituting 3-amino-1-methyl-pyridin-2-one with methyl 2-aminopyrimidine-4-carboxylate and completing the following two steps.

### Step 1:

To a solution of methyl (R)-2-((4-((1-(tert-butoxycarbonyl)piperidin-3-yl)(8-methylisoquinolin-1-yl)carbamoyl)-3-fluorophenyl) amino) pyrimidine-4-carboxylate (60 mg, 0.098 mmol) in THF (5 mL) was added NaBH₄ (18.46 mg, 0.488 mmol) at 0° C., and the reaction was stirred for 1 h. The reaction mixture was quenched with ice water and extracted with EtOAc (2×20 mL). The combined organic layers were washed with brine, dried over Na₂SO₄, and concentrated. The residue was purified by prep HPLC (Conditions: Luna OMEGA C-18 (250×21.2×5 μm), Mobile phase A: 0.1% formic acid in water, Mobile phase B: MeCN (100%), Gradient: Time (min)/% B: 0/60, 10/80, Flow: 15 mL/min, Dilution: MeCN+H2O+THF. The pure fractions were combined and concentrated to afford tert-butyl (R)-3-(2-fluoro-4-((4-(hydroxymethyl)pyrimidin-2-yl)amino)-N-(8-methylisoquinolin-1-yl)benzamido)piperidine-1-carboxylate (30 mg, 66.7%) as an off-white solid. LC-MS: [M+H⁺]=587.3.

### Step 2:

The title compound hydrochloride salt was obtained as an off-white solid by deprotection Method A used in Example 97. LC-MS: [M+H⁺]=487.30. ¹H NMR (CD₃OD, 400 MHZ) δ 8.46-8.40 (m, 2H), 7.75-7.69 (m, 1H), 7.67-7.63 (m, 1H), 7.50-7.43 (m, 3H), 7.04-7.03 (m, 1H), 6.84-6.78 (m, 1H), 6.68-6.64 (m, 1H), 4.98-4.92 (m, 1H), 4.54 (s, 2H), 3.96-3.93 (m, 1H), 3.83-3.73 (m, 1H), 3.51-3.35 (m, 1H), 3.02-2.94 (m, 1H), 2.74 (s, 3H), 1.94-1.86 (m, 3H), 1.58-1.55 (m, 1H).

### Synthesis of (R)-2-fluoro-4-((5-(hydroxymethyl)pyrimidin-2-yl)amino)-N-(8-methylisoquinolin-1-yl)-N-(piperidin-3-yl)benzamide (Example 322)

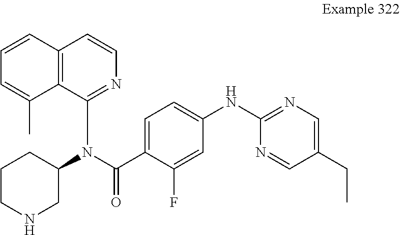 [SMILES: CCc1cnc(Nc2ccc(C(=O)N(c3nccc4cccc(C)c34)[C@@H]3CCCNC3)c(F)c2)nc1]

Example 322 was synthesized following the procedure for Example 321 by substituting methyl 2-aminopyrimidine-4-carboxylate with methyl 2-aminopyrimidine-5-carboxylate, provided the title compound hydrochloride salt as an off-white solid. LC-MS: [M+H⁺]=487.25. ¹H NMR (DMSO-d₆, 400 MHZ): δ 8.56 (m, 2H), 8.45-8.41 (m, 1H), 7.79-7.74 (m, 1H), 7.69-7.65 (m, 1H), 7.55-7.47 (m, 2H), 7.41-7.34 (m, 1H), 6.89-6.87 (m, 1H), 6.82-6.78 (m, 1H), 5.08-4.97 (m, 1H), 4.56 (s, 2H), 3.99-3.96 (m, 1H), 3.85-3.79 (m, 1H), 3.56-3.38 (m, 1H), 3.06-2.97 (m, 1H), 2.82 (s, 3H), 2.71-2.64 (m, 1H), 1.95-1.87 (m, 2H), 1.60-1.53 (m, 1H).

### Synthesis of (R)-2-fluoro-4-(3-methyl-1H-pyrazol-5-yl)-N-(8-methylisoquinolin-1-yl)-N-(piperidin-3-yl) benzamide (Example 323)

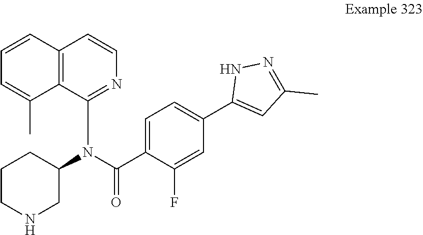 [SMILES: Cc1cc(-c2ccc(C(=O)N(c3nccc4cccc(C)c34)[C@@H]3CCCNC3)c(F)c2)[nH]n1]

Example 323 was prepared according to the procedure described for Example 127 by substituting 2-bromo-5-methyl-1,3,4-thiadiazole with (1-(tert-butoxycarbonyl)-3-methyl-1H-pyrazol-5-yl)boronic acid, provided the title compound hydrochloride salt as an off-white solid. LC-MS: [M+H⁺]=444.20. ¹H NMR (CD₃OD, 400 MHZ): δ 8.43-8.42 (m, 1H), 7.74-7.62 (m, 2H), 7.53-7.49 (m, 2H), 7.29-7.23 (m, 1H), 7.15-7.13 (m, 1H), 7.02-7.00 (m, 1H), 6.72 (s, 1H), 5.07-5.04 (m, 1H), 4.02-3.99 (m, 1H), 3.57-3.38 (m, 2H), 3.03-2.99 (m, 1H), 2.88 (s, 3H), 2.72-2.70 (m, 1H), 2.39 (s, 3H), 1.99-1.90 (m, 2H), 1.56-1.54 (m, 1H).

### Synthesis of (R)-2-fluoro-4-(3-methyl-1H-1,2,4-triazol-5-yl)-N-(8-methylisoquinolin-1-yl)-N-(piperidin-3-yl) benzamide (Example 324)

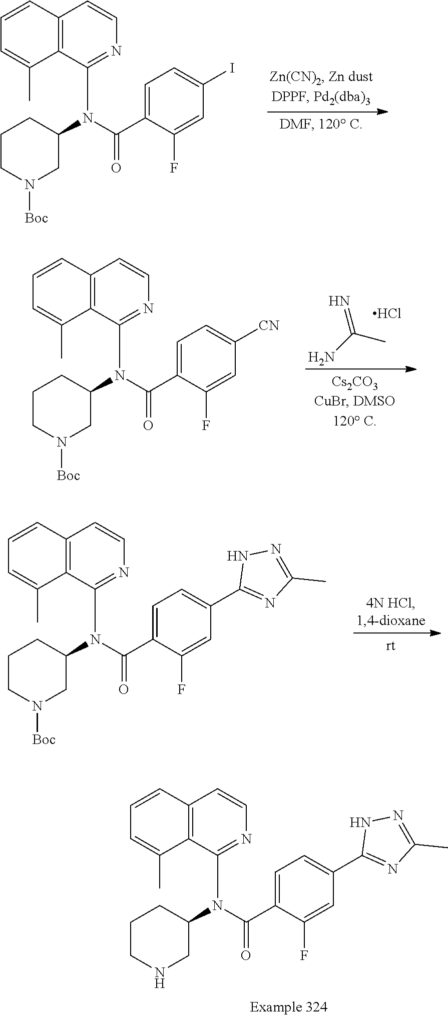

To a solution of tert-butyl (R)-3-(2-fluoro-4-iodo-N-(8-methylisoquinolin-1-yl)benzamido)piperidine-1-carboxylate (200 mg, 0.339 mmol) in DMF (3 mL) were added zinc(II) cyanide (80 mg, 0.679 mmol), zinc dust (4.44 mg, 0.068 mmol), and DPPF (7.52 mg, 0.014 mmol) at room temperature. The reaction mixture was degassed with nitrogen for 10 minutes, and then Pd₂(dba)₃ (12.43 mg, 0.014 mmol) was added to the reaction mixture. The reaction was stirred at 120° C. for 16 h. The reaction mixture was cooled to room temperature, diluted with water (20 mL) and extracted with ethyl acetate (2×30 mL). The combined organic layers were dried over Na₂SO₄ and concentrated. The residue was purified by Combi-flash using 30% EtOAc in hexane to afford tert-butyl (R)-3-(4-cyano-2-fluoro-N-(8-methylisoquinolin-1-yl) benzamido) piperidine-1-carboxylate (150 mg, 85%) as an off-white solid. LC-MS: [M−100+1]=389.30.

### Step 2:

To a solution of tert-butyl (R)-3-(4-cyano-2-fluoro-N-(8-methylisoquinolin-1-yl)benzamido)piperidine-1-carboxylate (150 mg, 0.307 mmol) in DMSO (4 mL) were added acetimidamide HCl (58.1 mg, 0.614 mmol), Cs₂CO₃ (300 mg, 0.921 mmol), and copper(I) bromide (44.0 mg, 0.307 mmol) at room temperature. The reaction mixture was stirred at 130° C. for 16 h. The reaction mixture was cooled to room temperature, diluted with ethyl acetate (15 mL), passed through Celite, and the celite bed was washed with ethyl acetate (20 mL). The combined organic layers were washed with water (20 mL) and brine (10 mL), dried by Na₂SO₄, and concentrated. The residue was purified by Combi-flash using 5% MeOH in DCM, and further purified by prep HPLC (Conditions: Kinetex Evo C-18 (250×21.2×5 μm), Mobile phase A: 0.1% formic acid in water (100%), Mobile phase B: 100% MeCN, Gradient: Time (min)/% B: 0/35, 10/60 Flow: 15 mL/min, Dilution: MeCN+H₂O+THF). The pure fractions were concentrated to afford tert-butyl (R)-3-(2-fluoro-4-((4-methyl-4H-1,2,4-triazol-3-yl)amino)-N-(8-methylisoquinolin-1-yl)benzamido)piperidine-1-carboxylate (35 mg, 29%) as an off-white solid. LC-MS: [M+H⁺]=545.25.

### Step 3:

The title compound hydrochloride salt was obtained as an off-white solid by deprotection Method A used in Example 97. LC-MS: [M+H⁺]=445.20. ¹H NMR (CD₃OD, 400 MHZ): δ 8.43-8.40 (m, 1H), 7.73-7.69 (m, 1H), 7.65-7.62 (m, 1H), 7.53-7.48 (m, 2H), 7.43-7.40 (m, 1H), 7.33-7.31 (m, 1H), 7.08-7.03 (m, 1H), 5.09-5.03 (m, 1H), 4.03-4.00 (m, 1H), 3.84-3.78 (m, 1H), 3.42-3.31 (m, 1H), 3.03-2.97 (m, 1H), 2.85 (s, 3H), 2.63 (s, 3H), 1.97-1.92 (m, 3H), 1.57-1.52 (m, 1H).

### Synthesis of (R)-4-(3H-[1,2,3]triazolo[4,5-b]pyridin-3-yl)-2-fluoro-N-(1,7-naphthyridin-8-yl)-N-(piperidin-3-yl)benzamide (Example 325)

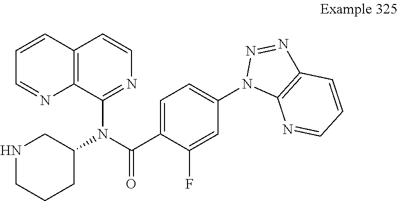 [SMILES: O=C(c1ccc(-n2nnc3cccnc32)cc1F)N(c1nccc2cccnc12)[C@@H]1CCCNC1]

Example 325 was synthesized following the procedure for Example 241 by substituting 6-bromo-1-chloroisoquinoline with 8-chloro-1,7-naphthyridine providing the title compound as a solid. LC-MS: [M+H⁺]=468.90. ¹H NMR (CD₃OD, 400 MHZ): δ 8.96 (s, 1H), 8.63 (d, J=4.4 Hz, 1H), 8.44-8.38 (m, 2H), 8.14 (d, J=8.4 Hz, 1H), 7.88 (d, J=11.6 Hz, 1H), 7.77 (d, J=8.4 Hz, 1H), 7.70 (d, J=5.6 Hz, 1H), 7.57-7.54 (m, 1H), 7.43-7.40 (m, 1H), 7.29-7.25 (m, 1H), 5.11-4.82 (m, 1H), 3.81-3.61 (m, 2H), 2.80-2.76 (m, 1H), 1.87-1.74 (m, 4H), 1.51-1.17 (m, 1H).

### Synthesis of (R)-3,4-bis(1-methyl-1H-pyrazol-4-yl)-N-(8-methylisoquinolin-1-yl)-N-(piperidin-3-yl) benzamide (Example 326)

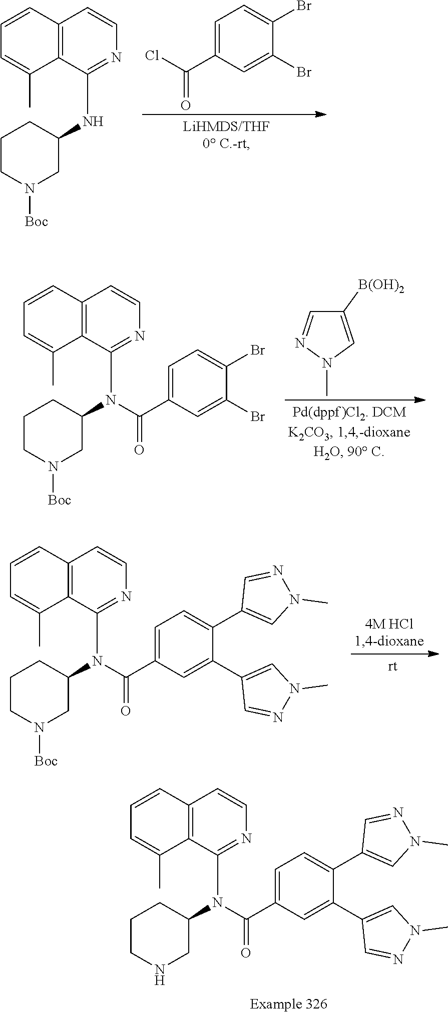

### Step 1:

To a solution of tert-butyl (R)-3-((8-methylisoquinolin-1-yl)amino)piperidine-1-carboxylate (500 mg, 1.464 mmol) in THF (10 mL) was added LiHMDS in THF (0.9 ml, 1.464 mmol) at 0° C., and the reaction was stirred for 10 min and followed by addition of 3,4-dibromobenzoyl chloride (655 mg, 2.196 mmol). The reaction was stirred for 30 min before it was quenched with cold water and extracted with EtOAc (2×50 mL). The combined organic layer was washed with brine, dried over Na₂SO₄, and concentrated. The residue was purified by Combi-flash using 80% EtOAc in hexane first and further purified by prep HPLC. (Conditions: Luna OMEGA C-18 (250×21.2×5 μm), Mobile phase A: 0.1% formic acid in water (100%), Mobile phase B: 100% MeCN, Gradient: Time (min)/% B: 0/75, 10/95, Flow: 15 mL/min, Dilution: MeCN+H₂O+THF). The pure fractions were concentrated to afford tert-butyl (R)-3-(3,4-dibromo-N-(8-methylisoquinolin-1-yl)benzamido)piperidine-1-carboxylate (250 mg, 55%) as a white solid.

### Step 2:

To a solution of tert-butyl (R)-3-(3,4-dibromo-N-(8-methylisoquinolin-1-yl)benzamido)piperidine-1-carboxylate (150 mg, 0.249 mmol) in 1,4-dioxane (5 mL) and H₂O (2 mL) were added (1-methyl-1H-pyrazol-4-yl)boronic acid (125 mg, 0.994 mmol) and K₂CO₃ (172 mg, 1.243 mmol) at room temperature. The reaction mixture was purged with nitrogen for 10 minutes, and PdCl₂(dppf) DCM (20.30 mg, 0.025 mmol) was added. The reaction was heated at 90° C. and stirred for 16 h in a sealed tube. The reaction mixture was diluted with EtOAc (50 mL), filtered through Celite, and the filtrate was washed with water (30 mL). The organic layer was dried over Na₂SO₄ and concentrated. The residue was purified by Combi-flash using 70% EtOAc in hexane, and then further purified by prep HPLC (Conditions: Luna OMEGA C-18 (250×21.2×5 μm), Mobile phase A: 0.1% formic acid in water (100%), Mobile phase B: 100% MeCN, Gradient: T/% B: 0/50, 10/85, Flow: 15 mL/min, Dilution: MeCN+H₂O+THF). The pure fractions were concentrated to afford tert-butyl (R)-3-(3,4-bis(1-methyl-1H-pyrazol-4-yl)-N-(8-methylisoquinolin-1-yl) benzamido) piperidine-1-carboxylate (30 mg, 33%) as a white solid. LC-MS: [M+H⁺]=606.30.

### Step 3:

The title compound hydrochloride salt was obtained as a white solid by deprotection Method A used in Example 97. LC-MS: [M+H⁺]=506.30. ¹H NMR (CD₃OD, 400 MHZ): δ 8.51-8.45 (m, 1H), 7.87-7.82 (m, 1H), 7.76-7.73 (m, 1H), 7.55-7.52 (m, 2H), 7.45-7.37 (m, 3H), 7.21 (s, 1H), 7.07 (s, 2H), 6.99 (s, 1H), 5.12-5.06 (m, 1H), 3.91 (s, 3H), 3.86 (s, 3H), 3.81-3.75 (m, 1H), 3.47-3.36 (m, 1H), 3.00-2.92 (m, 1H), 2.76 (s, 3H), 2.68-2.65 (m, 1H), 1.94-2.65 (m, 2H), 1.46-1.39 (m, 2H).

### Synthesis of 4-cyano-N-(8-methyl-1-isoquinolyl)-3-(4-phenylphenyl)-N-[(3R)-3-piperidyl]benzamide (Example 327)

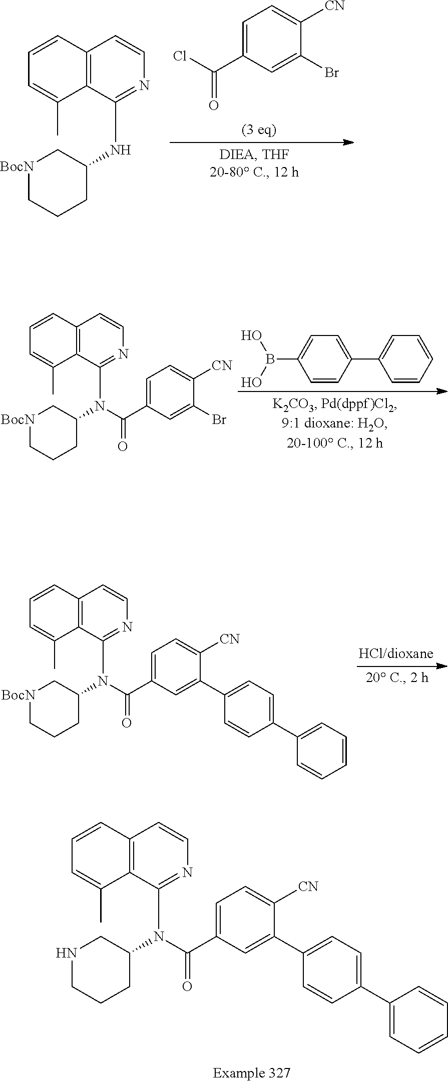 [SMILES: Cc1cccc2ccnc(N[C@@H]3CCCN(C(=O)OC(C)(C)C)C3)c12 ; N#Cc1ccc(C(=O)Cl)cc1Br ; Cc1cccc2ccnc(N(C(=O)c3ccc(C#N)c(Br)c3)[C@@H]3CCCN(C(=O)OC(C)(C)C)C3)c12 ; OB(O)c1ccc(-c2ccccc2)cc1 ; Cc1cccc2ccnc(N(C(=O)c3ccc(C#N)c(-c4ccc(-c5ccccc5)cc4)c3)[C@@H]3CCCN(C(=O)OC(C)(C)C)C3)c12 ; Cc1cccc2ccnc(N(C(=O)c3ccc(C#N)c(-c4ccc(-c5ccccc5)cc4)c3)[C@@H]3CCCNC3)c12]

### Step 1:

To a solution of tert-butyl (3R)-3-[(8-methyl-1-isoquinolyl)amino]piperidine-1-carboxylate (1 g, 2.93 mmol) in THF (20 mL) were added DIEA (1.89 g, 14.64 mmol, 2.55 mL) and 3-bromo-4-cyano-benzoyl chloride (1.99 g, 7.32 mmol) at 20° C. under N₂. The mixture was stirred at 80° C. for 12 hours. The reaction mixture was concentrated, and the residue was diluted with water (20 mL) and extracted with ethyl acetate (3×30 mL). The combined organic phases were washed with brine (50 mL), dried over anhydrous Na₂SO₄, and concentrated. The residue was purified by prep-HPLC (neutral condition; column: Waters Xbridge BEH C18 100*30 mm*10 um; mobile phase: [H₂O (10 mM NH₄HCO₃)-MeCN]; gradient: 45%-75% B over 8.0 min), and the product was concentrated to give tert-butyl (3R)-3-[(3-bromo-4-cyano-benzoyl)-(8-methyl-1-isoquinolyl)amino]piperidine-1-carboxylate (0.45 g, 26%) as a light yellow oil.

### Step 2:

To a solution of tert-butyl (3R)-3-[(3-bromo-4-cyano-benzoyl)-(8-methyl-1-isoquinolyl)amino]piperidine-1-carboxylate (100 mg, 172.90 μmol) and (4-phenylphenyl) boronic acid (102.71 mg, 518.69 μmol) in dioxane (3.6 mL) and H₂O (0.4 mL) were added K₂CO₃ (71.69 mg, 518.69 μmol) and Pd(dppf)Cl₂ (12.65 mg, 17.29 μmol) in order at 20° C. under N₂. The reaction was stirred at 100° C. for 12 hours. The reaction mixture was poured into water (10 mL) and extracted with ethyl acetate (3×30 mL). The combined organic phases were washed with brine (100 mL), dried over anhydrous Na₂SO₄, and concentrated to give a residue that was purified by prep-TLC (SiO₂, Petroleum ether:Ethyl acetate=1:1) to give tert-butyl (3R)-3-[[4-cyano-3-(4-phenylphenyl)benzoyl]-(8-methyl-1-isoquinolyl)amino]piperidine-1-carboxylate (90 mg, 50%) as a light brown oil.

### Step 3:

The title compound hydrochloride salt was obtained as an off-white solid by deprotection Method A used in Example 97. LC-MS: [M+H⁺]=523.4. ¹H NMR (400 MHZ, DMSO-d₆): δ 9.50-9.30 (m, 1H), 9.18 (br d, J=10.8 Hz, 1H), 8.58-8.48 (m, 1H), 7.99-7.88 (m, 1H), 7.84 (d, J=8.1 Hz, 1H), 7.80-7.70 (m, 3H), 7.69-7.64 (m, 1H), 7.61 (t, J=7.6 Hz, 1H), 7.50 (q, J=7.3 Hz, 3H), 7.45-7.39 (m, 1H), 7.34-7.27 (m, 1H), 7.19-7.12 (m, 2H), 7.04-6.98 (m, 1H), 5.08-4.80 (m, 1H), 3.78 (br d, J=10.6 Hz, 1H), 3.55 (q, J=10.9 Hz, 1H), 3.30-3.14 (m, 1H), 2.91-2.80 (m, 1H), 2.79-2.70 (m, 3H), 2.04-1.71 (m, 3H), 1.41-1.22 (m, 1H).

### Synthesis of 4-cyano-N-(8-methyl-1-isoquinolyl)-N-[(3R)-3-piperidyl]-3-[4-(3-pyridyl)phenyl]benzamide (Example 328)

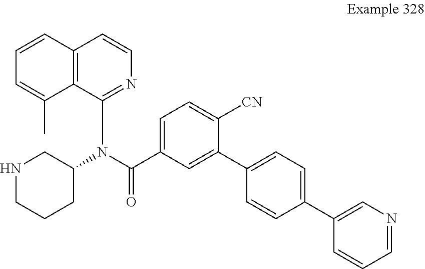 [SMILES: Cc1cccc2ccnc(N(C(=O)c3ccc(C#N)c(-c4ccc(-c5cccnc5)cc4)c3)[C@@H]3CCCNC3)c12]

Example 328 was prepared following the procedure for Example 327 by substituting (4-phenylphenyl) boronic acid with [4-(3-pyridyl)phenyl]boronic acid, provided the title compound hydrochloride salt as an off-white solid. LC-MS: [M+H⁺]=524.4. ¹H NMR (400 MHz, DMSO-d₆): δ 9.64 (br d, J=10.3 Hz, 1H), 9.36-9.17 (m, 2H), 8.84 (d, J=5.1 Hz, 1H), 8.71 (br d, J=8.0 Hz, 1H), 8.57-8.48 (m, 1H), 8.03-7.89 (m, 3H), 7.83 (d, J=8.1 Hz, 1H), 7.69 (d, J=8.0 Hz, 1H), 7.61 (t, J=7.6 Hz, 1H), 7.52-7.46 (m, 1H), 7.33 (dd, J=1.6, 8.1 Hz, 1H), 7.29-7.20 (m, 1H), 7.04 (d, J=1.4 Hz, 1H), 5.08-4.90 (m, 1H), 3.77 (br d, J=10.9 Hz, 1H), 3.60-3.49 (m, 1H), 3.23 (br d, J=12.0 Hz, 1H), 2.89 (br s, 1H), 2.79-2.71 (m, 3H), 1.98-1.69 (m, 3H), 1.40-1.22 (m, 1H).

### Synthesis of (R)-6-cyano-N-(8-methylisoquinolin-1-yl)-N-(piperidin-3-yl)-3′-(1H-pyrazol-4-yl)-[1,1′-biphenyl]-3-carboxamide (Example 329)

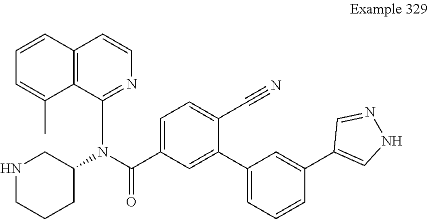 [SMILES: Cc1cccc2ccnc(N(C(=O)c3ccc(C#N)c(-c4cccc(-c5cn[nH]c5)c4)c3)[C@@H]3CCCNC3)c12]

Example 329 was synthesized following the procedure for Example 327 by substituting (4-phenylphenyl) boronic acid with 1-(tetrahydro-2H-pyran-2-yl)-4-(3-(4,4,5,5-tetramethyl-1,3,2-dioxaborolan-2-yl)phenyl)-1H-pyrazole, provided the title compound hydrochloride salt as a white solid. LC-MS: [M+H⁺]=513.4. ¹H NMR (400 MHz, DMSO-d₆): δ 9.25-8.73 (m, 2H), 8.65-8.46 (m, 1H), 8.20-8.06 (m, 2H), 8.04-7.93 (m, 1H), 7.89 (br d, J=8.0 Hz, 1H), 7.79-7.60 (m, 3H), 7.59-7.40 (m, 3H), 7.38-7.19 (m, 1H), 7.14-7.00 (m, 1H), 6.93-6.70 (m, 1H), 5.11-4.77 (m, 1H), 3.90-3.85 (m, 1H), 3.35-3.29 (m, 2H), 2.98-2.88 (m, 1H), 2.86-2.77 (m, 3H), 2.07-1.69 (m, 3H), 1.45-1.26 (m, 1H).

### Synthesis of 4-cyano-N-(8-methyl-1-isoquinolyl)-N-[(3R)-3-piperidyl]-3-[4-(4-pyridyl)phenyl]benzamide (Example 330)

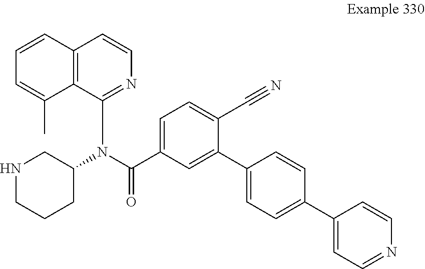 [SMILES: Cc1cccc2ccnc(N(C(=O)c3ccc(C#N)c(-c4ccc(-c5ccncc5)cc4)c3)[C@@H]3CCCNC3)c12]

Example 330 was synthesized following the procedure for Example 327 by substituting (4-phenylphenyl) boronic acid with [4-(4-pyridyl)phenyl]boronic acid, provided the title compound hydrochloride salt as an off-white solid. LC-MS: [M+H⁺]=524.3. ¹H NMR (400 MHZ, DMSO-d₆) δ 9.40-9.61 (m, 1H), 9.09-9.29 (m, 1H), 8.99 (d, J=6.50 Hz, 2H), 8.49-8.57 (m, 1H), 8.40 (d, J=6.25 Hz, 2H), 8.13 (d, J=8.38 Hz, 2H), 7.89-7.95 (m, 1H), 7.83 (d, J=8.00 Hz, 1H), 7.67-7.74 (m, 1H), 7.56-7.65 (m, 1H), 7.45-7.53 (m, 1H), 7.27-7.38 (m, 3H), 7.01-7.13 (m, 1H), 4.91-5.07 (m, 1H), 3.78 (br d, J=11.26 Hz, 2H), 3.50-3.59 (m, 1H), 3.20-3.30 (m, 1H), 2.71-2.77 (m, 3H), 1.69-2.00 (m, 3H), 1.30 (m, 4.13 Hz, 1H).

### Synthesis of (R)-4-(3H-[1,2,3]triazolo[4,5-b]pyridin-3-yl)-2-fluoro-N-(6-(3-hydroxyprop-1-en-1-yl)isoquinolin-1-yl)-N-(piperidin-3-yl)benzamide (Example 331)

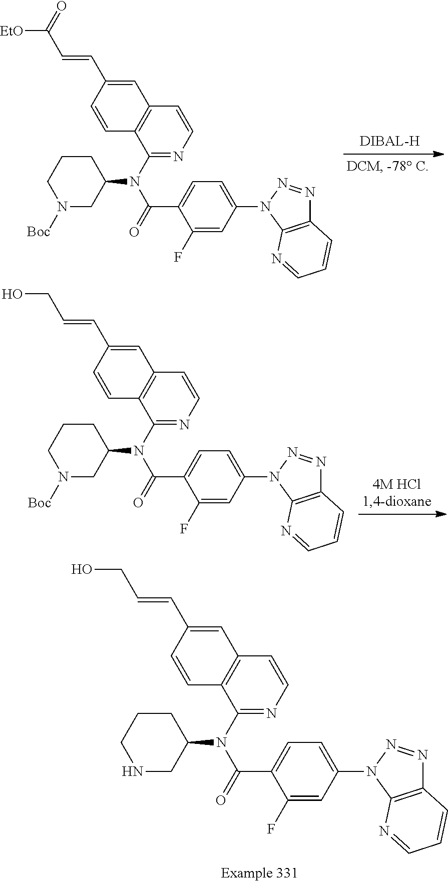 [SMILES: CCOC(=O)/C=C/c1ccc2c(N(C(=O)c3ccc(-n4nnc5cccnc54)cc3F)[C@@H]3CCCN(C(=O)OC(C)(C)C)C3)nccc2c1 ; CC(C)(C)OC(=O)N1CCC[C@@H](N(C(=O)c2ccc(-n3nnc4cccnc43)cc2F)c2nccc3cc(/C=C/CO)ccc23)C1 ; O=C(c1ccc(-n2nnc3cccnc32)cc1F)N(c1nccc2cc(/C=C/CO)ccc12)[C@@H]1CCCNC1]

### Step 1:

To a solution of tert-butyl (R,E)-3-(4-(3H-[1,2,3]triazolo[4,5-b]pyridin-3-yl)-N-(6-(3-ethoxy-3-oxoprop-1-en-1-yl)isoquinolin-1-yl)-2-fluorobenzamido) piperidine-1-carboxylate (100 mg, 0.150 mmol) in DCM (5 mL) was added DIBAL-H (21.36 mg, 0.150 mmol) at −78° C., and the reaction was stirred at room temperature for 30 minutes. The reaction mixture was diluted with water (15 mL) and extracted with DCM (2×30 mL). The organic layers were combined and washed with brine, dried by Na₂SO₄, and concentrated. The residue was purified by Combi-flash using 80% EtOAc in hexane. The residue was further purified by prep HPLC (Conditions: Luna OMEGA C-18 (250×21.2×5 μm), Mobile phase A: 0.1% formic acid in water (100%), Mobile phase B: 100% MeCN, Gradient: Time (min)/% B: 0/30, 10/75, Flow: 15 mL/min, Dilution: MeCN+H₂O+THF). The pure fractions were dried by lyophilization to afford tert-butyl (R,E)-3-(4-(3H-[1,2,3]triazolo[4,5-b]pyridin-3-yl)-N-(6-(3-ethoxy-3-oxoprop-1-en-1-yl)isoquinolin-1-yl)-2-fluorobenzamido) piperidine-1-carboxylate (45 mg, 0.07 mmol, 55%) as an off-white solid. LC-MS: [M+H⁺]=624.30.

### Step 2:

The title compound hydrochloride salt was obtained as an off-white solid by deprotection Method A used in Example 97. LC-MS: [M+H⁺]=524.25. ¹H NMR (CD₃OD, 400 MHZ): δ 8.74 (d, J=4.4 Hz, 1H), 8.49 (d, J=8.4 Hz, 1H), 8.42 (d, J=6.0 Hz, 1H), 8.12-8.10 (m, 1H), 8.04 (dd, J=1.6 Hz, J=10.8 Hz, 1H), 7.92-7.88 (m, 2H), 7.73-7.70 (m, 2H), 7.53-7.50 (m, 1H), 7.33-7.29 (m, 1H), 6.76-6.72 (m, 1H), 6.67-6.59 (m, 1H), 5.29-5.23 (m, 1H), 4.28-4.27 (m, 2H), 3.97-3.94 (m, 1H), 3.71-3.65 (m, 1H), 3.35-3.27 (m, 1H), 2.90-2.82 (m, 1H), 2.03-2.00 (m, 1H), 1.91-1.85 (m, 2H), 1.30-1.26 (m, 1H).

### Synthesis of (R)—N-(8-methylisoquinolin-1-yl)-4-(2-oxo-1,2-dihydropyridin-3-yl)-N-(piperidin-3-yl)benzamide (Example 332)

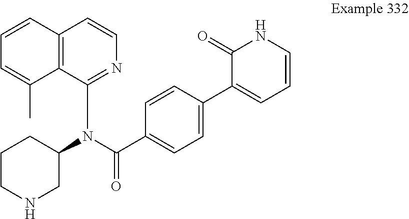 [SMILES: Cc1cccc2ccnc(N(C(=O)c3ccc(-c4ccc[nH]c4=O)cc3)[C@@H]3CCCNC3)c12]

Example 332 was prepared according to the procedure described for Example 127 by substituting 2-bromo-5-methyl-1,3,4-thiadiazole with (2-oxo-1,2-dihydropyridin-3-yl)boronic acid, provided the title compound hydrochloride salt as a white solid. LC-MS: [M+H⁺]=439.2. ¹H NMR (CD₃OD, 400 MHZ): δ 8.5 (d, J=5.6 Hz, 1H), 7.9-7.79 (m, 1H), 7.72-7.69 (m, 1H), 7.55-7.48 (m, 2H), 7.41-7.38 (m, 2H), 7.35-7.33 (m, 2H), 7.12-7.10 (m, 2H), 6.45-6.42 (m, 1H), 5.11-5.04 (m, 1H), 3.95-3.92 (m, 1H), 3.80-3.74 (m, 1H), 3.47-3.35 (m, 1H), 2.99-2.91 (m, 2H), 2.85 (s, 3H), 2.72-2.65 (m, 1H), 1.92-1.90 (m, 2H).

### Synthesis of 4-cyano-N-(8-methyl-1-isoquinolyl)-N-[(3R)-3-piperidyl]-2-(1H-pyrazol-5-yl)benzamide (Example 333)

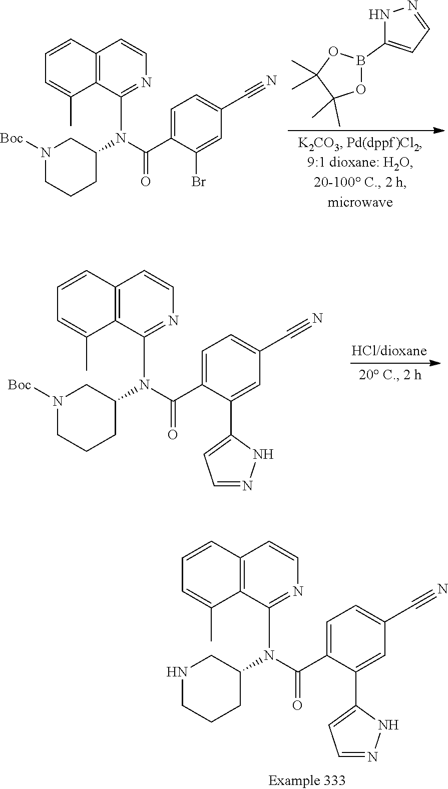

### Step 1:

To a solution of tert-butyl (3R)-3-[(2-bromo-4-cyano-benzoyl)-(8-methyl-1-isoquinolyl)amino]piperidine-1-carboxylate (100 mg, 172.90 μmol) and 1H-pyrazol-5-ylboronic acid (193.46 mg, 1.73 mmol) in dioxane (3.6 mL) and H₂O (0.4 mL) were added K₂CO₃ (71.69 mg, 518.69 μmol) and Pd(dppf)Cl₂ (12.65 mg, 17.29 μmol) in order at 20° C. under N₂ in a microwave tube. The mixture was stirred at 100° C. for 2 h under microwave conditions. The reaction mixture was then poured into water (10 mL) and extracted with ethyl acetate (3×30 mL). The combined organic phases were washed with brine (3×30 mL), dried over anhydrous Na₂SO₄, and concentrated. The residue was purified by prep-TLC (SiO₂, Petroleum ether:Ethyl acetate=1:1) to afford tert-butyl (3R)-3-[[4-cyano-2-(1H-pyrazol-5-yl)benzoyl]-(8-methyl-1-isoquinolyl)amino]piperidine-1-carboxylate (80 mg, 34%) as a light brown oil.

The title compound hydrochloride salt was obtained as an off-white solid by deprotection Method A used in Example 97. LC-MS: [M+H⁺]=437.3. ¹H NMR (400 MHZ, DMSO-d₆): δ 9.06-8.90 (m, 1H), 8.87-8.72 (m, 1H), 8.56-8.44 (m, 1H), 8.07-7.90 (m, 2H), 7.88-7.70 (m, 2H), 7.67-7.59 (m, 2H), 7.68-7.48 (m, 2H), 7.44-7.37 (m, 1H), 7.44-7.27 (m, 1H), 6.47 (br s, 1H), 4.89-4.73 (m, 1H), 4.38-4.26 (m, 1H), 4.15-3.95 (m, 1H), 3.25-3.07 (m, 2H), 2.78-2.68 (m, 3H), 1.95-1.63 (m, 3H), 1.40-1.19 (m, 1H).

### Synthesis of 4-cyano-N-(8-methyl-1-isoquinolyl)-N-[(3R)-3-piperidyl]-2-(3-pyridyl)benzamide (Example 334)

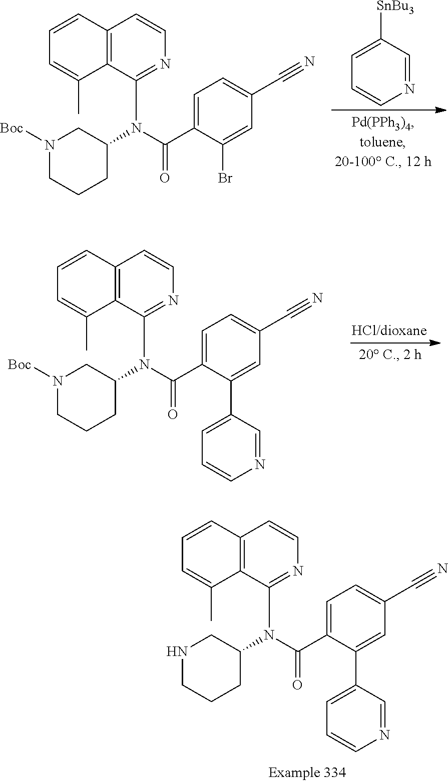

### Step 1:

To a solution of tert-butyl (3R)-3-[(2-bromo-4-cyano-benzoyl)-(8-methyl-1-isoquinolyl)amino]piperidine-1-carboxylate (50 mg, 81.90 μmol) and tributyl(3-pyridyl)stannane (90.45 mg, 245.70 μmol) in toluene (2 mL) was added Pd(PPh₃)₄ (28.39 mg, 24.57 μmol) at 20° C. under Ar. The mixture was stirred at 100° C. for 12 h. The reaction mixture was poured into water (10 mL) and extracted with ethyl acetate (3×10 mL). The combined organic phases were washed with brine (30 mL), dried with anhydrous Na₂SO₄, and concentrated. The residue was purified by prep-HPLC (HCl condition, column: Phenomenex Luna C18 100*40 mm*5 μm; mobile phase: [H₂O (0.04% HCl)-MeCN]; gradient: 30%-60% B over 8.0 min) and the product was lyophilized to afford tert-butyl (3R)-3-[[4-cyano-2-(3-pyridyl)benzoyl]-(8-methyl-1-isoquinolyl)amino]piperidine-1-carboxylate hydrochloride salt (20 mg, 42%) as a light yellow oil.

### Step 2:

The title compound hydrochloride salt was obtained as an off-white solid by deprotection Method A used in Example 97. LC-MS: [M+H⁺]=448.3. ¹H NMR (400 MHZ, DMSO-d₆): δ 8.75 (br s, 3H), 8.57-8.49 (m, 1H), 8.10 (d, J=5.4 Hz, 1H), 7.83-7.75 (m, 3H), 7.66-7.52 (m, 4H), 7.43 (dd, J=1.4, 8.1 Hz, 1H), 7.28 (d, J=8.1 Hz, 1H), 4.74-4.49 (m, 1H), 3.15-3.06 (m, 2H), 2.92-2.83 (m, 1H), 2.76-2.70 (m, 4H), 1.78-1.57 (m, 3H), 1.12-0.92 (m, 1H).

### Synthesis of 4-cyano-N-(8-methyl-1-isoquinolyl)-N-[(3R)-3-piperidyl]-2-(2-pyridyl)benzamide (Example 335)

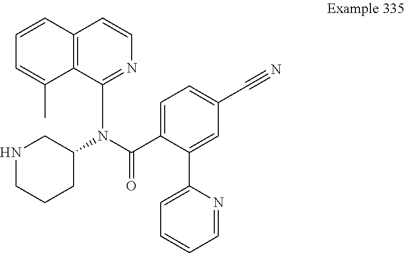 [SMILES: Cc1cccc2ccnc(N(C(=O)c3ccc(C#N)cc3-c3ccccn3)[C@@H]3CCCNC3)c12]

Example 335 was prepared following the procedure for Example 334 by substituting tributyl(3-pyridyl)stannane with tributyl(2-pyridyl)stannane, provided the title compound hydrochloride salt as an off-white solid. LC-MS: [M+H⁺]=448.2. ¹H NMR (400 MHZ, DMSO-d₆): δ 9.48 (br d, J=9.4 Hz, 1H), 9.29-9.01 (m, 1H), 8.78-8.66 (m, 1H), 7.93 (dt, J=1.7, 7.8 Hz, 1H), 7.81-7.34 (m, 10H), 4.97-4.80 (m, 1H), 4.02 (br d, J=10.9 Hz, 1H), 3.20 (br d, J=11.4 Hz, 1H), 3.07-2.91 (m, 1H), 2.78-2.63 (m, 4H), 1.89-1.53 (m, 3H), 1.31-1.10 (m, 1H).

### Synthesis of 4-cyano-N-(8-methyl-1-isoquinolyl)-N-[(3R)-3-piperidyl]-3-[3-(3-pyridyl)phenyl]benzamide (Example 336)

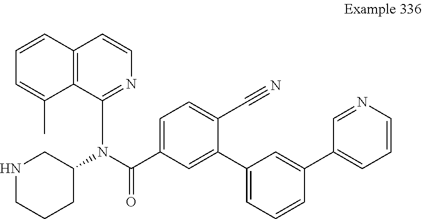 [SMILES: Cc1cccc2ccnc(N(C(=O)c3ccc(C#N)c(-c4cccc(-c5cccnc5)c4)c3)[C@@H]3CCCNC3)c12]

Example 336 was synthesized following the procedure for Example 327 by substituting (4-phenylphenyl) boronic acid with [3-(3-pyridyl)phenyl]boronic acid, provided the title compound hydrochloride salt as an off-white solid. LC-MS: [M+H⁺]=524.3. ¹H NMR (400 MHZ, DMSO-d₆) δ 9.92-8.98 (m, 3H), 8.97-8.59 (m, 2H), 8.51-8.42 (m, 1H), 8.08-7.84 (m, 3H), 7.80 (br d, J=8.1 Hz, 1H), 7.74-7.60 (m, 2H), 7.59-7.43 (m, 3H), 7.38-7.27 (m, 1H), 7.26-7.08 (m, 2H), 5.03-4.86 (m, 1H), 3.84-3.82 (m, 1H), 3.30-3.20 (m, 2H), 2.84-2.69 (m, 4H), 1.89-1.66 (m, 3H), 1.43-1.19 (m, 1H).

### Synthesis of (R,Z)-4-(3H-[1,2,3]triazolo[4,5-b]pyridin-3-yl)-2-fluoro-N-(7-(3-hydroxyprop-1-en-1-yl)isoquinolin-1-yl)-N-(piperidin-3-yl)benzamide (Example 337)

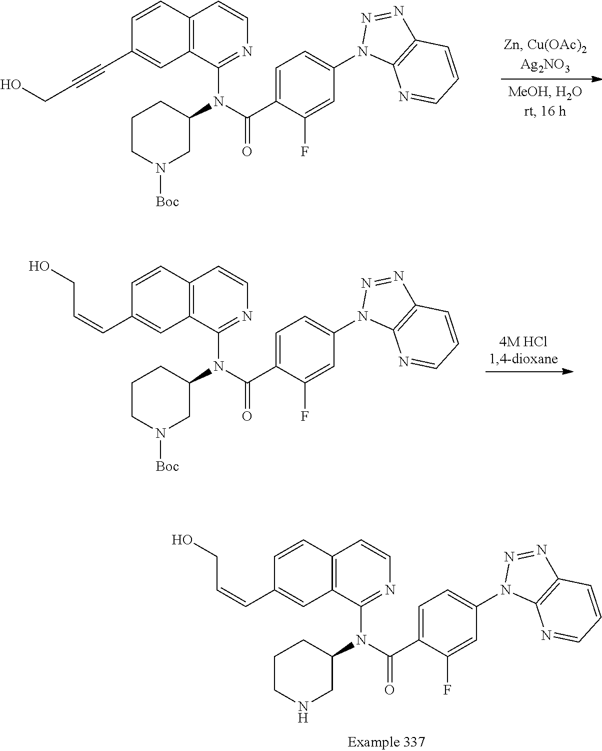 [SMILES: CC(C)(C)OC(=O)N1CCC[C@@H](N(C(=O)c2ccc(-n3nnc4cccnc43)cc2F)c2nccc3ccc(C#CCO)cc23)C1 ; CC(C)(C)OC(=O)N1CCC[C@@H](N(C(=O)c2ccc(-n3nnc4cccnc43)cc2F)c2nccc3ccc(/C=C\CO)cc23)C1 ; O=C(c1ccc(-n2nnc3cccnc32)cc1F)N(c1nccc2ccc(/C=C\CO)cc12)[C@@H]1CCCNC1]

### Step 1:

A solution of zinc (2945 mg, 45 mmol) in water (3 mL) was stirred under nitrogen for 15 minutes. Then copper (II) acetate (266 mg, 1.464 mmol) was added, and the reaction was stirred for 15 minutes. Silver nitrate (287 mg, 1.689 mmol) was added to the reaction mixture, and the reaction was again stirred at room temperature for 30 minutes. The mixture was filtered and washed successively with water, methanol, acetone, and diethyl ether. The resulting solid was added to a stirred solution of tert-butyl (R)-3-(4-(3H-[1,2,3]triazolo[4,5-b]pyridin-3-yl)-2-fluoro-N-(7-(3-hydroxyprop-1-yn-1-yl)isoquinolin-1-yl)benzamido)-piperidine-1-carboxylate (70 mg, 0.113 mmol) in MeOH (3 mL), and the reaction was stirred at room temperature for 1 h. The reaction mixture was filtered through a bed of celite, which was then washed with ethyl acetate (10 mL). The filtrate was concentrated under reduced pressure. The residue was purified by prep HPLC (Conditions: X-SELECT CSH, Mobile phase A: 0.1% formic acid in water (100%), Mobile phase B: 100% MeCN, Gradient: T/% B: 0/65, 10/85, Flow: 15 mL/min, Dilution: THF+MeCN+H₂O). Pure fractions were dried by lyophilization to afford tert-butyl (R,Z)-3-(4-(3H-[1,2,3]triazolo[4,5-b]pyridin-3-yl)-2-fluoro-N-(7-(3-hydroxyprop-1-en-1-yl)isoquinolin-1-yl)benzamido)piperidine-1-carboxylate formate salt (15 mg) as an off-white solid. LC-MS: [M+H⁺]=624.3.

### Step 2:

The title compound hydrochloride salt was obtained as an off-white solid by deprotection Method A used in Example 97. LC-MS: [M+H⁺]=524.0. ¹H NMR (CD₃OD, 400 MHZ): δ 8.75-8.74 (m, 1H), 8.52-8.39 (m, 2H), 8.06-7.98 (m, 2H), 7.91-7.80 (m, 2H), 7.75-7.74 (m, 1H), 7.68-7.60 (m, 1H), 7.54-7.52 (m, 1H), 7.30-7.26 (m, 1H), 6.78-6.75 (m, 1H), 6.14-6.07 (m, 1H), 5.30-5.26 (m, 1H), 4.50-4.37 (m, 2H), 3.99-3.95 (m, 1H), 3.75-3.70 (m, 1H), 2.93-2.88 (m, 1H), 2.06-1.99 (m, 3H), 1.39-1.28 (m, 1H), 0.94-0.89 (m, 1H).

### Synthesis of (R,Z)—N-(8-(3-hydroxyprop-1-en-1-yl)isoquinolin-1-yl)-4-(1-methyl-1H-1,2,3-triazol-4-yl)-N-(piperidin-3-yl)benzamide (Example 338)

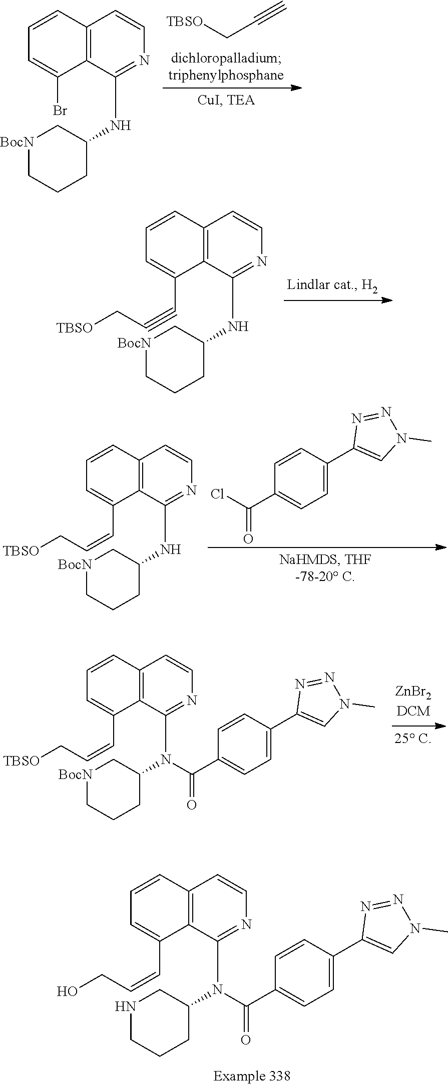

### Step 1:

A mixture of tert-butyl (3R)-3-[(8-bromo-1-isoquinolyl) amino] piperidine-1-carboxylate (300 mg, 738.34 μmol), tert-butyl-dimethyl-prop-2-ynoxy-silane (1.26 g, 7.38 mmol), dichloropalladium; triphenylphosphane (51.82 mg, 73.83 μmol), CuI (14.06 mg, 73.83 μmol) and TEA (205.54 μL, 1.48 mmol) in MeCN (5 mL) at 20° C. was degassed and purged with N₂ for 3 times, and then the mixture was heated to 84° C. and stirred for 12 h under N₂ atmosphere. The reaction mixture was concentrated under reduced pressure to give a residue. The residue was purified by prep-TLC (SiO₂, petroleum ether/ethyl acetate=2:1) to give tert-butyl (3R)-3-[[8-[3-[tert-butyl(dimethyl)silyl] oxyprop-1-ynyl]-1-isoquinolyl] amino] piperidine-1-carboxylate (213 mg, 58%) as a yellow oil.

### Step 2:

A mixture of tert-butyl (3R)-3-[[8-[3-[tert-butyl(dimethyl)silyl] oxyprop-1-ynyl]-1-isoquinolyl] amino] piperidine-1-carboxylate (198 mg, 399.41 μmol) in MeOH (5 mL) was added Lindlar catalyst (1.65 g, 798.83 μmol, 10% wt) at 20° C. under N₂, and then the mixture was degassed and purged with H₂ for 3 times at 20° C. The mixture was stirred at 20° C. for 12 h under H₂ atmosphere (15 Psi). The mixture was filtered to give a filtrate. The filtrate was concentrated under reduced pressure to give a residue. The residue was purified by pre-TLC (SiO₂, petroleum ether/ethyl acetate=1:1) to give tert-butyl (3R)-3-[[8-[(Z)-3-[tert-butyl(dimethyl)silyl] oxyprop-1-enyl]-1-isoquinolyl] amino] piperidine-1-carboxylate (188 mg, 94%) as a yellow oil.

### Step 3:

A mixture of 4-(1-methyltriazol-4-yl) benzoic acid (70 mg, 344.49 μmol) in SOCl₂ (125.10 μL, 1.72 mmol) was degassed and purged with N₂ for 3 times at 20° C., and then the mixture was heated to 80° C. and stirred for 1 h under N₂ atmosphere. The reaction mixture was concentrated to give a crude 4-(1-methyltriazol-4-yl)benzoyl chloride (70 mg, 91%) as a white solid.

### Step 4:

To a solution of tert-butyl (3R)-3-[[8-[(Z)-3-[tert-butyl(dimethyl)silyl] oxyprop-1-enyl]-1-isoquinolyl] amino] piperidine-1-carboxylate (50 mg, 100.45 μmol) in THF (2 mL) was added a solution of 1M NaHMDS (120.54 μL) at −78° C. under N₂ atmosphere. The mixture was stirred at −40° C. for 0.5 h. Then the mixture was added 4-(1-methyltriazol-4-yl) benzoyl chloride (66.79 mg, 301.36 μmol) at 0° C. under N₂ atmosphere. The mixture was stirred at 20° C. for 2 h. The reaction mixture was quenched by addition water 10 mL at 20° C., and then extracted with ethyl acetate 30 mL (3×10 mL). The combined organic layer was washed with brine 30 mL (3×10 mL), dried over Na₂SO₄, filtered, and concentrated. The residue was purified by prep-TLC (SiO₂, petroleum ether:ethyl acetate=1:3) to give tert-butyl (3R)-3-[[8-[(Z)-3-[tert-butyl(dimethyl)silyl]oxyprop-1-enyl]-1-isoquinolyl]-[4-(1-methyltriazol-4-yl)benzoyl]amino]piperidine-1-carboxylate (30 mg, 43%) as a yellow solid.

### Step 5:

To a solution of tert-butyl (3R)-3-[[8-[(Z)-3-[tert-butyl(dimethyl)silyl] oxyprop-1-enyl]-1-isoquinolyl]-[4-(1-methyltriazol-4-yl)benzoyl] amino] piperidine-1-carboxylate (25 mg, 36.61 μmol) in DCM (1 mL) at 25° C. was added ZnBr₂ (41.22 mg, 183.04 μmol). The mixture was stirred at 25° C. for 12 h. The reaction mixture was filtered and concentrated to give a residue. The residue was purified by Prep-HPLC (Conditions: Waters Xbridge Prep OBD C18 150*40 mm*10 μm column; mobile phase: [H₂O (10 mM NH₄HCO₃)-MeCN]; gradient: 15%-50% B over 8.0 min) to provide N-[8-[(Z)-3-hydroxyprop-1-enyl]-1-isoquinolyl]-4-(1-methyltriazol-4-yl)-N-[(3R)-3-piperidyl] benzamide (10 mg, 58%) as an off-white solid. LC-MS: [M+H⁺]=469.2. ¹H NMR (400 MHZ, CD₃OD): δ 8.53-8.38 (m, 2H), 8.14-8.06 (m, 1H), 8.01-7.93 (m, 2H), 7.87-7.81 (m, 2H), 7.68-7.56 (m, 1H), 7.49-7.19 (m, 2H), 6.41-6.21 (m, 1H), 4.63-4.59 (m, 1H), 4.41-4.36 (m, 1H), 4.19 (s, 3H), 3.67 (t, J=5.9 Hz, 1H), 3.39-3.33 (m, 1H), 3.23 (br dd, J=2.6, 12.5 Hz, 1H), 3.01-2.84 (m, 1H), 2.81-2.66 (m, 2H), 2.20-2.02 (m, 1H), 1.88-1.75 (m, 2H), 1.70-1.60 (m, 1H).

### Synthesis of 2-(4-((S)-1-((8-methylisoquinolin-1-yl)((R)-piperidin-3-yl)carbamoyl)piperidin-3-yl)phenyl)-2H-indazole-7-carboxamide (Example 339)

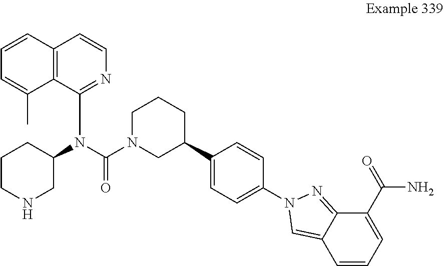 [SMILES: Cc1cccc2ccnc(N(C(=O)N3CCC[C@@H](c4ccc(-n5cc6cccc(C(N)=O)c6n5)cc4)C3)[C@@H]3CCCNC3)c12]

Example 339 was prepared following the procedure for Example 243 by substituting 3-methyl-5-(piperidin-4-yl)isoxazole with(S)-2-(4-(piperidin-3-yl)phenyl)-2H-indazole-7-carboxamide, provided the title compound hydrochloride salt as an off-white solid. LC-MS: [M+H⁺]=588.3. ¹H NMR (CD₃OD, 400 MHz): δ 8.95 (s, 1H), 8.39 (d, J=6.0 Hz, 1H), 8.15 (d, J=7.2 Hz, 1H), 8.02 (d, J=8.4 Hz, 1H), 7.93-7.83 (m, 4H), 7.78-7.72 (m, 1H), 7.63-7.60 (m, 1H), 7.28-7.24 (m, 1H), 7.21-7.14 (m, 2H), 4.67-4.62 (m, 1H), 3.99-3.93 (m, 1H), 3.73-3.58 (m, 2H), 3.34-3.30 (m, 1H), 2.95-2.90 (m, 1H), 2.81 (s, 3H), 2.72-2.63 (m, 1H), 2.54-2.46 (m, 2H), 2.14-2.11 (m, 1H), 2.00-1.97 (m, 2H), 1.86-1.71 (m, 3H), 1.52-1.49 (m, 1H), 1.32-1.28 (m, 1H), 1.01-0.98 (m, 1H).

### Synthesis of (R)-3-(4-amino-3-(4-phenoxyphenyl)-1H-pyrazolo[3,4-d]pyrimidin-1-yl)-N-(8-methylisoquinolin-1-yl)-N—((R)-piperidin-3-yl)piperidine-1-carboxamide (Example 340)

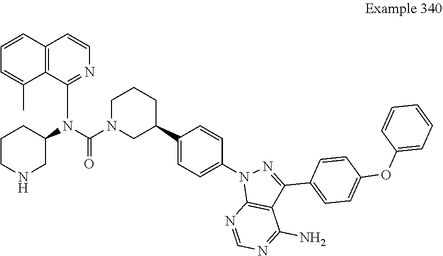 [SMILES: Cc1cccc2ccnc(N(C(=O)N3CCC[C@@H](c4ccc(-n5nc(-c6ccc(Oc7ccccc7)cc6)c6c(N)ncnc65)cc4)C3)[C@@H]3CCCNC3)c12]

Example 340 was prepared according to the procedure described for Example 243 by substituting 3-methyl-5-(piperidin-4-yl)isoxazole with (R)-3-(4-phenoxyphenyl)-1-(piperidin-3-yl)-1H-pyrazolo[3,4-d]pyrimidin-4-amine, provided the title compound as an off-white solid. LC-MS: [M+H⁺]=654.2. ¹H NMR (CD₃OD, 400 MHZ): δ 8.46-8.44 (m, 1H), 8.37 (d, J=5.6 Hz, 1H), 7.86-7.84 (m, 1H), 7.78-7.59 (m, 5H), 7.44-7.39 (m, 2H), 7.21-7.09 (m, 5H), 4.95-4.79 (m, 1H), 4.29-4.25 (m, 1H), 3.94-3.84 (m, 2H), 3.66-3.65 (m, 1H), 3.53-3.47 (m, 1H), 3.07-3.04 (m, 2H), 2.90-2.87 (m, 2H), 2.75 (s, 3H), 2.55-2.20 (m, 1H), 1.97-1.82 (m, 3H), 1.77-1.74 (m, 3H), 1.38-1.21 (m, 1H).

### Synthesis of N-(8-methylisoquinolin-1-yl)-3-(5-phenyl-1H-imidazol-2-yl)-N—((R)-piperidin-3-yl)piperidine-1-carboxamide (Example 341)

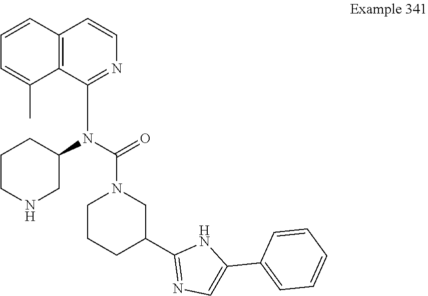 [SMILES: Cc1cccc2ccnc(N(C(=O)N3CCCC(c4ncc(-c5ccccc5)[nH]4)C3)[C@@H]3CCCNC3)c12]

Example 341 was prepared according to the procedure described for Example 243 by substituting 3-methyl-5-(piperidin-4-yl)isoxazole with (R)-2-(piperidin-3-yl) isoindoline-1,3-dione, provided the title compound hydrochloride salt as an off-white solid. LC-MS: [M+H⁺]=495.3. ¹H NMR (CD₃OD, 400 MHZ): δ 8.39-8.21 (m, 1H), 7.82-7.80 (m, 1H), 7.79-7.71 (m, 5H), 7.54-7.52 (m, 4H), 3.92-3.84 (m, 1H), 3.74-3.65 (m, 2H), 3.22-3.12 (m, 1H), 2.92-2.89 (m, 4H), 2.76 (s, 3H), 2.51-2.34 (m, 1H), 2.00-1.94 (m, 2H), 1.85-1.76 (m, 4H), 1.28-1.16 (m, 1H), 0.94-0.89 (m, 1H), 0.35-0.26 (m, 1H).

### Synthesis of benzyl (R)-2-((8-methylisoquinolin-1-yl)(piperidin-3-yl)carbamoyl)-2,9-diazaspiro [5.5]undecane-9-carboxylate (Example 342)

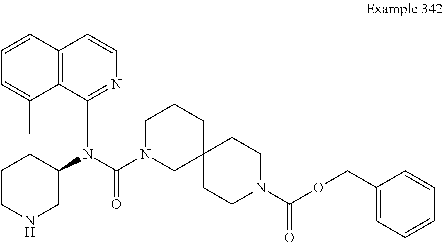 [SMILES: Cc1cccc2ccnc(N(C(=O)N3CCCC4(CCN(C(=O)OCc5ccccc5)CC4)C3)[C@@H]3CCCNC3)c12]

Example 342 was prepared according to the procedure described for Example 243 by substituting 3-methyl-5-(piperidin-4-yl)isoxazole with benzyl 2,9-diazaspiro[5.5]undecane-9-carboxylate, provided the title compound hydrochloride salt as an off-white solid. LC-MS: [M+H⁺]=556.35. ¹H NMR (CD₃OD, 400 MHZ): δ 8.37-8.26 (m, 1H), 7.85-7.79 (m, 1H), 7.78-7.73 (m, 1H), 7.68-7.61 (m, 1H), 7.57-7.48 (m, 1H), 7.39-7.32 (m, 5H), 5.10 (s, 2H), 4.91-4.84 (m, 1H), 3.82-3.79 (m, 1H), 3.65-3.60 (m, 1H), 3.37-3.30 (m, 3H), 3.13-3.10 (m, 3H), 2.68 (s, 3H), 2.38-2.27 (m, 2H), 2.06-1.94 (m, 1H), 1.84-1.73 (m, 3H), 1.28-0.90 (m, 10H).

### Synthesis of (R)—N-(8-methylisoquinolin-1-yl)-3-phenyl-N-(piperidin-3-yl)-1,4,6,7-tetrahydro-5H-pyrazolo[4,3-c]pyridine-5-carboxamide (Example 343)

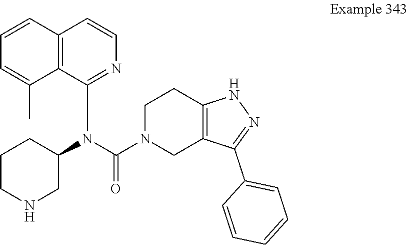 [SMILES: Cc1cccc2ccnc(N(C(=O)N3CCc4[nH]nc(-c5ccccc5)c4C3)[C@@H]3CCCNC3)c12]

Example 343 was prepared according to the procedure described for Example 243 by substituting 3-methyl-5-(piperidin-4-yl) isoxazole with 3-phenyl-4,5,6,7-tetrahydro-1H-pyrazolo[4,3-c]pyridine, provided the title compound hydrochloride salt as an off-white solid. LC-MS: [M+H⁺]=467.3. ¹H NMR (CD₃OD, 400 MHZ): δ 8.22-8.06 (m, 1H), 7.75-7.71 (m, 1H), 7.61-7.54 (m, 2H), 7.50-7.48 (m, 2H), 7.43-7.32 (m, 2H), 7.26-7.24 (m, 1H), 7.11-7.09 (m, 1H), 4.84-4.79 (m, 1H), 4.25-4.09 (m, 1H), 3.83-3.58 (m, 4H), 3.47-3.40 (m, 1H), 3.21-3.12 (m, 1H), 2.93-2.86 (m, 2H), 2.70 (s, 3H), 2.57-2.46 (m, 1H), 2.38-2.22 (m, 1H), 2.11-1.92 (m, 1H), 1.80-1.78 (m, 2H).

### Synthesis of N-(8-methylisoquinolin-1-yl)-3-(3-oxo-3-(phenylamino) propyl)-N—((R)-piperidin-3-yl) piperidine-1-carboxamide (Example 344)

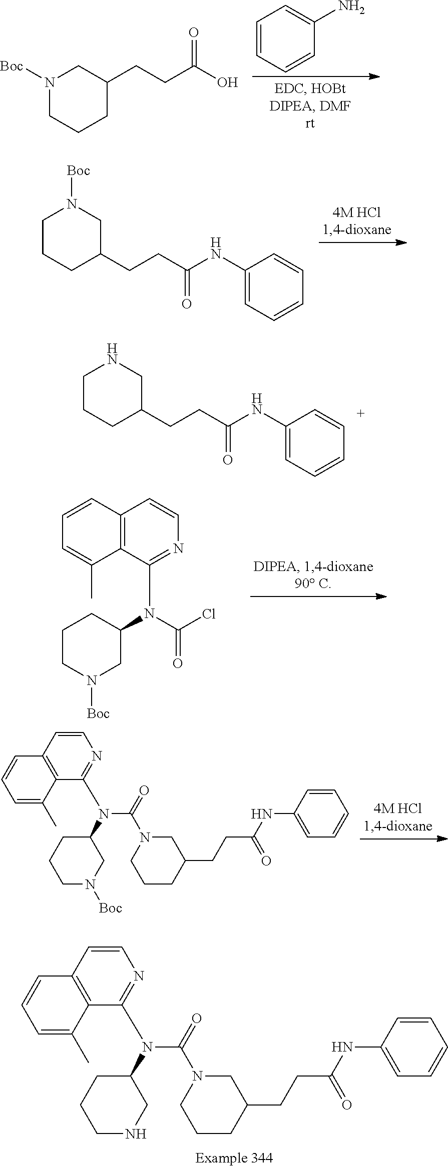

### Step 1:

To a solution of 3-(1-(tert-butoxycarbonyl)piperidin-3-yl) propanoic acid (200 mg, 0.777 mmol) and aniline (145 mg, 1.554 mmol) in DMF (2 ml) were added EDC-HCl (298 mg, 1.554 mmol), HOBt (238 mg, 1.554 mmol), and DIPEA (0.407 ml, 2.332 mmol). The reaction was stirred at room temperature for 16 h. The reaction mixture was diluted with water (50 mL) and extracted with EtOAc (2×50 mL). The organic layers were washed with brine, dried over Na₂SO₄, and concentrated. The residue was purified by Combi-flash using 60% EtOAc in hexane to afford tert-butyl 3-(3-oxo-3-(phenylamino) propyl) piperidine-1-carboxylate (120 mg, 46%) as a brown solid. LC-MS: [M−H]=331.15.

### Step 2:

To a solution of tert-butyl 3-(3-oxo-3-(phenylamino) propyl)piperidine-1-carboxylate (120 mg, 0.361 mmol) in DCM (1 mL) at 0° C. was added 4 M HCl in 1,4-dioxane (1 mL, 32.9 mmol). The reaction was warmed to 25° C. and stirred for 2 h. The reaction mixture was concentrated, washed with diethyl ether, and dried by lyophilization to afford N-phenyl-3-(piperidin-3-yl) propenamide hydrochloride salt (80 mg, 82%) as an off-white solid. LC-MS: [M+H⁺]=233.25.

### Steps 3-4:

Example 344 was prepared according to the procedure for Example 243 by substituting 3-methyl-5-(piperidin-4-yl) isoxazole with N-phenyl-3-(piperidin-3-yl) propenamide, provided the title compound hydrochloride salt as an off-white solid. LC-MS: [M+H⁺]=500.3. ¹H NMR (CD₃OD, 400 MHZ) δ 8.39-8.26 (m, 1H), 7.80-7.72 (m, 2H), 7.64-7.47 (m, 4H), 7.36-7.30 (m, 2H), 7.13-7.08 (m, 1H), 4.84-4.74 (m, 1H), 3.93-3.87 (m, 1H), 3.65-3.53 (m, 2H), 2.91-2.87 (m, 3H), 2.68-2.60 (m, 3H), 2.18-1.93 (m, 6H), 1.39-1.25 (m, 5H), 1.28-1.07 (m, 3H), 0.88-0.80 (m, 1H).

### Synthesis of 3-(1,3-dioxoisoindolin-2-yl)-N-(8-methylisoquinolin-1-yl)-N—((R)-piperidin-3-yl)piperidine-1-carboxamide (Example 345)

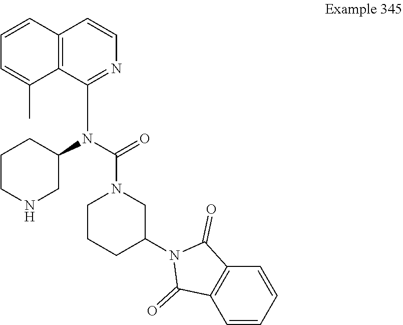 [SMILES: Cc1cccc2ccnc(N(C(=O)N3CCCC(N4C(=O)c5ccccc5C4=O)C3)[C@@H]3CCCNC3)c12]

Example 345 was prepared according to the procedure described for Example 243 by substituting 3-methyl-5-(piperidin-4-yl)isoxazole with(S)-2-(piperidin-3-yl) isoindoline-1,3-dioxane, provided the title compound hydrochloride salt as an off-white solid. LC-MS: [M+H⁺]=498.25. ¹H NMR (CD₃OD, 400 MHZ): δ 8.37-8.27 (m, 1H), 7.85-7.83 (m, 1H), 7.79-7.76 (m, 4H), 7.74-7.66 (m, 2H), 7.60-7.58 (m, 1H), 4.08-3.79 (m, 1H), 3.69-3.65 (m, 3H), 3.64-3.47 (m, 1H), 3.11-3.02 (m, 2H), 2.93-2.88 (m, 2H), 2.72 (s, 3H), 2.34-2.31 (m, 1H), 2.03-1.97 (m, 2H), 1.86-1.60 (m, 2H), 1.45-1.41 (m, 1H), 1.13-1.10 (m, 1H), 0.94-0.85 (m, 1H), 0.15-0.08 (m, 1H).

### Synthesis of N-[4-[(Z)-4-hydroxybut-1-enyl]-2-pyridyl]-4-(1-methyltriazol-4-yl)-N-[(3R)-3-piperidyl] benzamide (Example 346)

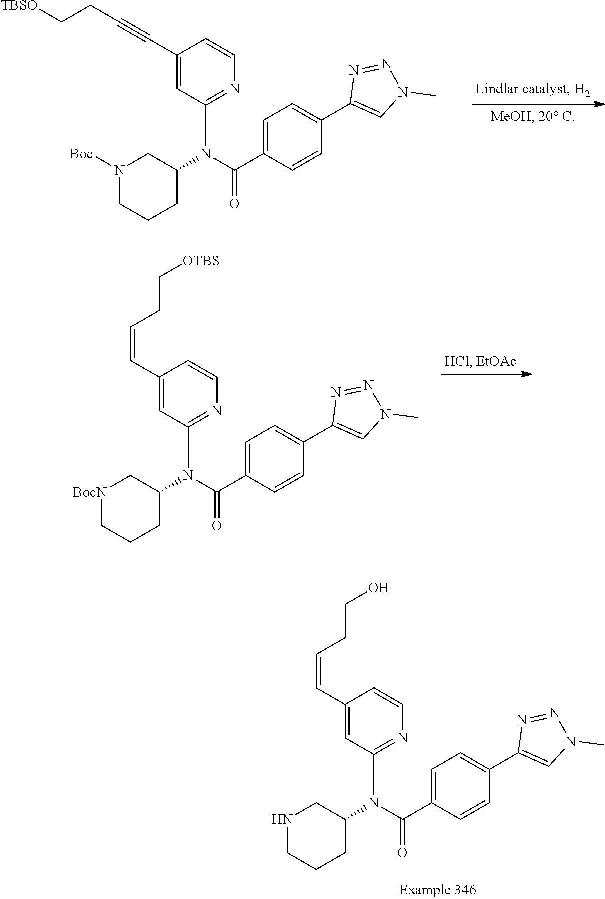 [SMILES: Cn1cc(-c2ccc(C(=O)N(c3cc(C#CCCO[Si](C)(C)C(C)(C)C)ccn3)[C@@H]3CCCN(C(=O)OC(C)(C)C)C3)cc2)nn1 ; Cn1cc(-c2ccc(C(=O)N(c3cc(/C=C\CCO[Si](C)(C)C(C)(C)C)ccn3)[C@@H]3CCCN(C(=O)OC(C)(C)C)C3)cc2)nn1 ; Cn1cc(-c2ccc(C(=O)N(c3cc(/C=C\CCO)ccn3)[C@@H]3CCCNC3)cc2)nn1]

### Step 1:

To a mixture of Lindlar catalyst (38.92 mg, 94.23 μmol) in MeOH (2 mL) was added tert-butyl (3R)-3-[[4-(4-hydroxybut-1-ynyl)-2-pyridyl]-[4-(1-methyltriazol-4-yl) benzoyl]amino]-piperidine-1-carboxylate (50 mg, 94.23 μmol) at 20° C. The reaction mixture was stirred at 20° C. for 24 h under H₂ at 15 psi. The reaction was filtered through a pad of Celite, and the filter cake was washed with methanol (3×20 mL). The filtrate was concentrated to give tert-butyl (R,Z)-3-(N-(4-(4-((tert-butyldimethylsilyl)oxy)but-1-en-1-yl)pyridin-2-yl)-4-(1-methyl-1H-1,2,3-triazol-4-yl)benzamido)piperidine-1-carboxylate (50 mg, 93%) as a yellow solid.

### Step 2:

The title compound hydrochloride salt was obtained as an off-white solid by deprotection Method A used in Example 97. LC-MS: [M+H⁺]=433.2. ¹H NMR (400 MHZ, DMSO-d₆): δ 9.16 (br d, J=10.1 Hz, 1H), 8.87 (br d, J=10.4 Hz, 1H), 8.53-8.34 (m, 2H), 7.74-7.63 (m, 2H), 7.34-6.91 (m, 4H), 6.35-5.76 (m, 1H), 5.01-4.79 (m, 1H), 4.07 (d, J=2.8 Hz, 3H), 3.61-3.46 (m, 2H), 3.33-3.10 (m, 3H), 2.74 (br d, J=10.9 Hz, 1H), 2.56 (br s, 1H), 2.03-0.98 (m, 6H).

### Synthesis of N-(8-methyl-1-isoquinolyl)-4-(1-methyltriazol-4-yl)-N-[(3R)-3-piperidyl]piperidine-1-carboxamide (Example 347)

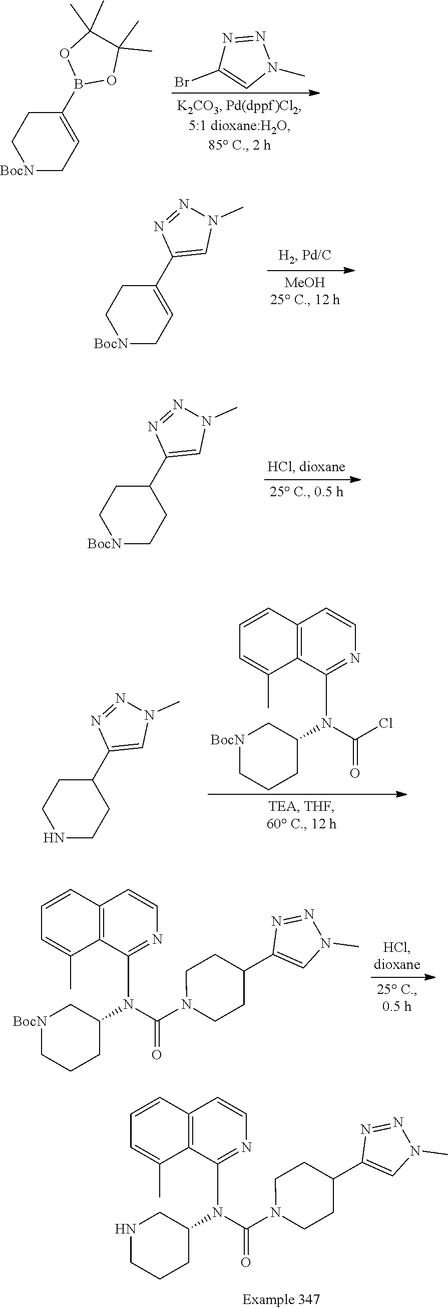

### Step 1:

A mixture of 4-bromo-1-methyl-triazole (523.88 mg, 3.23 mmol), tert-butyl 4-(4,4,5,5-tetramethyl-1,3,2-dioxaborolan-2-yl)-3,6-dihydro-2H-pyridine-1-carboxylate (1 g, 3.23 mmol), K₂CO₃ (1.34 g, 9.70 mmol), and Pd(dppf)Cl₂ (236.64 mg, 323.41 μmol) in dioxane (10 mL) and H₂O (2 mL) was degassed and purged with N₂ 3 times, and then the mixture was stirred at 85° C. for 2 h under N₂ atmosphere. The reaction mixture was poured into 50 mL water. Then the mixture was extracted with ethyl acetate (3×10 mL). The combined organic layer was washed with saturated brine (20 mL), dried over anhydrous Na₂SO₄, and concentrated. The residue was purified by column chromatography (SiO₂, Petroleum ether/Ethyl acetate=5/1 to 4/1) to give tert-butyl 4-(1-methyltriazol-4-yl)-3,6-dihydro-2H-pyridine-1-carboxylate (544 mg, 61%) as a white solid.

### Step 2:

To a solution of tert-butyl 4-(1-methyltriazol-4-yl)-3,6-dihydro-2H-pyridine-1-carboxylate (520 mg, 1.97 mmol) in MeOH (10 mL) under N₂ was added Pd/C (4.19 g, 3.93 mmol, 10% purity). The mixture was stirred at 25° C. for 12 h under H₂ (15 psi). The reaction mixture was filtered and the filtrate was concentrated to give tert-butyl 4-(1-methyltriazol-4-yl) piperidine-1-carboxylate (490 mg, 82%) as a white solid, which was used in the next step directly.

### Step 3:

To a solution of tert-butyl 4-(1-methyltriazol-4-yl)piperidine-1-carboxylate (480 mg, 1.80 mmol) in dioxane (5 mL) at 25° C. was added HCl in dioxane (4 M, 901 μL). The mixture was stirred at 25° C. for 1 h. The reaction mixture was concentrated to give 4-(1-methyltriazol-4-yl) piperidine (260 mg, 82%) as a white solid, which was used for the next step directly.

### Step 4:

To a solution of tert-butyl (3R)-3-[chlorocarbonyl-(8-methyl-1-isoquinolyl)amino] piperidine-1-carboxylate (220.90 mg, 546.91 μmol) in 1,2-dichloroethane (5 mL) were added TEA (553.42 mg, 5.47 mmol, 761.24 μL) and 4-(1-methyltriazol-4-yl) piperidine (100 mg, 601.60 μmol) at 25° C. under N₂. The mixture was stirred at 25° C. for 12 h, after which it was concentrated. The residue was purified by prep-HPLC (column: Waters Xbridge C18 150*50 mm*10 μm; mobile phase: [H₂O (10 mM NH₄HCO₃)-MeCN]; gradient: 35%-70% B over 8.0 min) to give tert-butyl (3R)-3-[(8-methyl-1-isoquinolyl)-[4-(1-methyltriazol-4-yl)piperidine-1-carbonyl]amino]-piperidine-1-carboxylate (47 mg, 16%) as a white solid. LC-MS: [M+H⁺]=534.4.

### Step 5:

The title compound hydrochloride salt was obtained as a white solid by deprotection Method A used in Example 97. LC-MS: [M+H⁺]=434.4. ¹H NMR (400 MHZ, CD₃OD): δ 8.44-8.31 (m, 1H), 8.28-8.15 (m, 1H), 7.97-7.89 (m, 2H), 7.81-7.73 (m, 1H), 7.71-7.60 (m, 1H), 4.77-4.67 (m, 1H), 4.26-4.21 (m, 3H), 4.14-3.82 (m, 2H), 3.79-3.64 (m, 2H), 3.59-3.47 (m, 1H), 3.36 (br d, J=10.6 Hz, 2H), 3.02-2.93 (m, 2H), 2.90-2.75 (m, 3H), 2.71-2.53 (m, 2H), 2.39-2.06 (m, 1H), 2.02-1.70 (m, 4H), 1.57-0.61 (m, 2H).

### Synthesis of (R,E)-N-(8-(2-(1H-pyrazol-4-yl)vinyl)isoquinolin-1-yl)-4-(1-methyl-1H-1,2,3-triazol-4-yl)-N-(piperidin-3-yl)benzamide (Example 348)

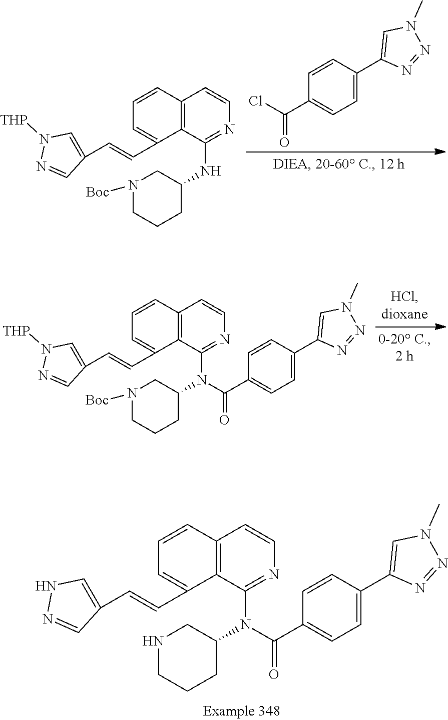

To a solution of tert-butyl (3R)-3-((8-((E)-2-(1-(tetrahydro-2H-pyran-2-yl)-1H-pyrazol-4-yl)vinyl)isoquinolin-1-yl)amino)piperidine-1-carboxylate (210 mg, 416.97 μmol) in THF (3 mL) were added DIEA (269.45 mg, 2.08 mmol, 363.14 μL) and 4-(1-methyltriazol-4-yl)benzoyl chloride (138.63 mg, 625.45 μmol) at 20° C. under N₂. The reaction was stirred at 60° C. for 12 h. The reaction mixture was filtered and concentrated to give a residue, which was purified by column chromatography (SiO₂, Petroleum ether/Ethyl acetate=5/1 to 3/1) to give tert-butyl (3R)-3-(4-(1-methyl-1H-1, 2,3-triazol-4-yl)-N-(8-((E)-2-(1-(tetrahydro-2H-pyran-2-yl)-1H-pyrazol-4-yl)vinyl)isoquinolin-1-yl)benzamido)piperidine-1-carboxylate (30 mg, 9%) as a white solid. LC-MS: [M+H⁺]=689.6.

### Step 2:

A solution of tert-butyl (3R)-3-(4-(1-methyl-1H-1, 2,3-triazol-4-yl)-N-(8-((E)-2-(1-(tetrahydro-2H-pyran-2-yl)-1H-pyrazol-4-yl)vinyl)isoquinolin-1-yl)benzamido)piperidine-1-carboxylate (30 mg, 43.55 μmol) in HCl/dioxane (2 mL) was stirred at 0° C. for 2 h under N₂. The reaction mixture was filtered and concentrated. The residue was purified by prep-HPLC (column: Waters Xbridge BEH C18 100*30 mm*10 μm; mobile phase: [A: H₂O (10 mM NH₄HCO₃); B: MeCN]; B %: 25.00%-55.00%, 8.00 min) to give (R,E)-N-(8-(2-(1H-pyrazol-4-yl)vinyl)isoquinolin-1-yl)-4-(1-methyl-1H-1,2,3-triazol-4-yl)-N-(piperidin-3-yl)benzamide (8.6 mg, 36%) as a white solid. LC-MS: [M+H⁺]=505.6. ¹H NMR (400 MHZ, CD₃OD): δ 8.50-8.47 (m, 1H), 8.09-7.99 (m, 3H), 7.77-7.63 (m, 3H), 7.54-7.47 (m, 2H), 7.36 (d, J=8.4 Hz, 2H), 7.04 (d, J=8.4 Hz, 2H), 6.90-6.84 (m, 1H), 4.65-4.60 (m, 1H), 4.05 (s, 3H), 3.67-3.64 (m, 1H), 3.40-3.31 (m, 1H), 3.01-2.98 (m, 1H), 2.56-2.44 (m, 1H), 1.87-1.69 (m, 3H), 1.38-1.31 (m, 1H).

### Synthesis of 4-(1-methyltriazol-4-yl)-N-[(3R)-3-piperidyl]-N-[8-[(E)-2-(3-pyridyl)vinyl]-1-isoquinolyl]benzamide (Example 349)

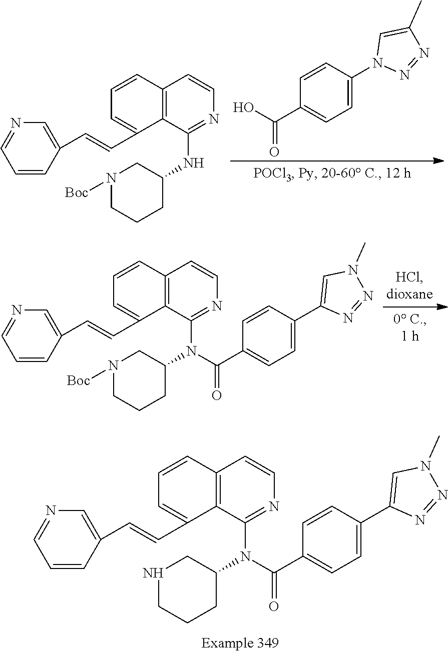

### Step 1:

To a solution of tert-butyl (3R)-3-[[8-[(E)-2-(3-pyridyl)vinyl]-1-isoquinolyl]amino]piperidine-1-carboxylate (170 mg, 394.85 μmol) in pyridine (3 mL) at 0° C. were added POCl₃ (60.54 mg, 394.85 μmol) and 4-(4-methyltriazol-1-yl)benzoic acid (96.28 mg, 473.82 μmol) under N₂. The reaction was heated to 60° C. and stirred for 12 h. The reaction mixture was poured into H₂O (5 mL) under N₂. And the aqueous phase was extracted with ethyl acetate (3×5 mL). The combined organic phases were washed with saturated brine (5 mL), dried over anhydrous Na₂SO₄, and concentrated. The residue was purified by Prep-HPLC [column: Phenomenex Luna C18 75*30 mm*3 μm; mobile phase: [H₂O (0.04% HCl)-MeCN]; gradient: 60%-85% B over 8.0 min]. Pure fractions were dried by lyophilization to afford tert-butyl (3R)-3-[[4-(1-methyltriazol-4-yl)benzoyl]-[8-[(E)-2-(3-pyridyl)vinyl]-1-isoquinolyl]amino]piperidine-1-carboxylate (30 mg, 12%) as a yellow solid. LC-MS: [M+H⁺]=616.3.

### Step 2:

The title compound hydrochloride salt was obtained as an off-white solid by deprotection Method A used in Example 97. LC-MS: [M+H⁺]=516.4. ¹H NMR (400 MHZ, CD₃OD): δ 9.41-9.24 (m, 1H), 9.20-9.04 (m, 1H), 8.93-8.82 (m, 1H), 8.66-8.55 (m, 1H), 8.26 (dd, J=5.8, 8.1 Hz, 1H), 8.16 (s, 1H), 8.11-7.99 (m, 1H), 7.91-7.82 (m, 2H), 7.72 (br d, J=7.0 Hz, 1H), 7.64 (t, J=7.8 Hz, 1H), 7.44-7.38 (m, 2H), 7.20-7.09 (m, 1H), 7.03-6.94 (m, 2H), 5.47-5.24 (m, 1H), 4.08 (s, 3H), 3.92-3.81 (m, 1H), 3.73-3.64 (m, 1H), 3.59-3.41 (m, 2H), 3.24-2.95 (m, 1H), 2.22-1.85 (m, 2H), 1.75-1.54 (m, 1H).

### Synthesis of (R)-4-(4-aminopyrimidin-5-yl)-2-fluoro-N-(8-methylisoquinolin-1-yl)-N-(piperidin-3-yl)benzamide (Example 353)

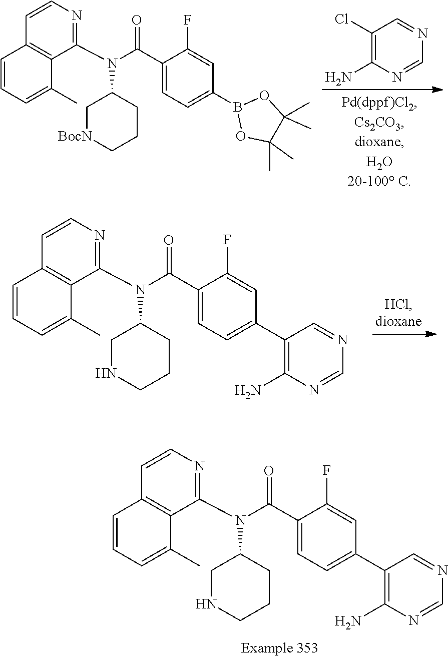

### Step 1:

To a solution of tert-butyl (R)-3-(2-fluoro-N-(8-methylisoquinolin-1-yl)-4-(4,4,5,5-tetramethyl-1,3,2-dioxaborolan-2-yl)benzamido)piperidine-1-carboxylate (0.10 g, 101.78 μmol) in dioxane (0.9 mL) and H₂O (0.1 mL) at 20° C. under N₂ atmosphere was added 5-chloropyrimidin-4-amine (26.37 mg, 203.56 μmol), Pd(dppf)Cl₂ (7.45 mg, 10.18 μmol) and Cs₂CO₃ (99.49 mg, 305.34 μmol). Then the reaction mixture was stirred at 100° C. for 12 h under N₂ atmosphere and then allowed to cool. The reaction mixture was filtrated through a pad of Celite. The filtrate was diluted with water (10 mL) and extracted with ethyl acetate (3×10 mL). The combined organic layers were washed with brine (10 mL), dried over Na₂SO₄, filtered and concentrated. The residue was purified by prep-HPLC (column: Phenomenex luna C18 100*40 mm*5 μm; mobile phase: [H₂O (0.04% HCl)-MeCN]; gradient: 20%-50% B over 8.0 min) and the pure fraction was lyophilized to give tert-butyl (R)-3-(4-(4-aminopyrimidin-5-yl)-2-fluoro-N-(8-methylisoquinolin-1-yl)benzamido)-piperidine-1-carboxylate (0.01 g, 17%) as a brown solid. LC-MS: [M+H⁺]=557.30.

### Step 2:

The title compound hydrochloride salt was obtained as an off-white solid by deprotection Method A used in Example 97. LC-MS: [M+H⁺]=457.20. ¹H NMR (400 MHZ, DMSO-d₆) δ 9.30-9.17 (m, 1H), 9.11-8.87 (m, 1H), 8.82-8.62 (m, 1H), 8.53-8.41 (m, 1H), 8.36-8.20 (m, 1H), 8.04-7.95 (m, 1H), 7.94-7.89 (m, 1H), 7.88-7.69 (m, 2H), 7.64-7.44 (m, 2H), 7.28-7.11 (m, 1H), 7.05-6.86 (m, 1H), 4.97-4.84 (m, 1H), 4.69-4.39 (m, 1H), 3.80-3.70 (m, 1H), 3.48 (br s, 1H), 2.97-2.93 (m, 1H), 2.86-2.73 (m, 3H), 1.95-1.55 (m, 3H), 1.23 (m, 1H).

### Synthesis of 4-(4,6-dimethylpyrimidin-5-yl)-2-fluoro-N-(8-methyl-1-isoquinolyl)-N-[(3R)-3-piperidyl]benzamide (Example 354)

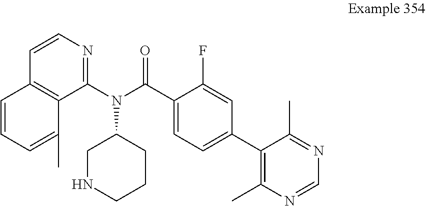 [SMILES: Cc1ncnc(C)c1-c1ccc(C(=O)N(c2nccc3cccc(C)c23)[C@@H]2CCCNC2)c(F)c1]

Example 354 was prepared according to the procedure described for Example 353 by substituting 5-chloropyrimidin-4-amine with 5-bromo-4,6-dimethyl-pyrimidine, providing the title compound as a yellow solid. LC-MS: [M+H⁺]=470.4. ¹H NMR (400 MHZ, CD₃OD): δ 9.27 (s, 1H), 8.47-8.40 (m, 1H), 7.78-7.71 (m, 1H), 7.70-7.63 (m, 1H), 7.58-7.44 (m, 2H), 7.04-6.95 (m, 1H), 6.85 (d, J=10.3 Hz, 1H), 6.69 (d, J=7.9 Hz, 1H), 5.19-5.06 (m, 1H), 4.09-3.99 (m, 1H), 3.98-3.86 (m, 1H), 3.45 (br d, J=12.3 Hz, 1H), 3.05 (dt, J=4.6, 11.9 Hz, 1H), 2.91-2.81 (m, 3H), 2.17 (s, 3H), 2.09 (s, 3H), 2.06-1.93 (m, 3H), 1.88-1.73 (m, 1H).

### Synthesis of (R)-4-(4-amino-2-methylpyridin-3-yl)-2-fluoro-N-(8-methylisoquinolin-1-yl)-N-(piperidin-3-yl)benzamide (Example 361)

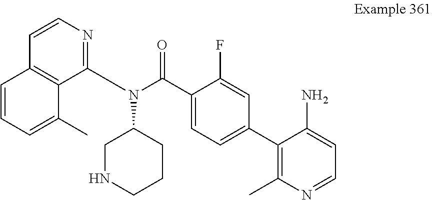 [SMILES: Cc1nccc(N)c1-c1ccc(C(=O)N(c2nccc3cccc(C)c23)[C@@H]2CCCNC2)c(F)c1]

Example 361 was prepared according to the procedure described for Example 353 by substituting 5-chloropyrimidin-4-amine with 3-bromo-2-methyl-pyridin-4-amine, provided the title compound hydrochloride salt as a dark brown solid. LC-MS: [M+H⁺]=470.4. ¹H NMR (400 MHZ, DMSO-d₆): δ 9.50-9.03 (m, 1H), 8.55-8.35 (m, 1H), 8.12-7.92 (m, 2H), 7.82-7.64 (m, 2H), 7.63-7.18 (m, 3H), 6.89-6.40 (m, 3H), 5.04-4.78 (m, 1H), 3.83-3.60 (m, 2H), 3.57 (br s, 3H), 3.36-3.24 (m, 1H), 2.82-2.65 (m, 3H), 2.00-1.83 (m, 2H), 1.63-1.42 (m, 3H).

### Synthesis of 3-allyl-N-(4-allyl-2-pyridyl)-4-chloro-N-[(3R)-3-piperidyl]benzamide (Example 362)

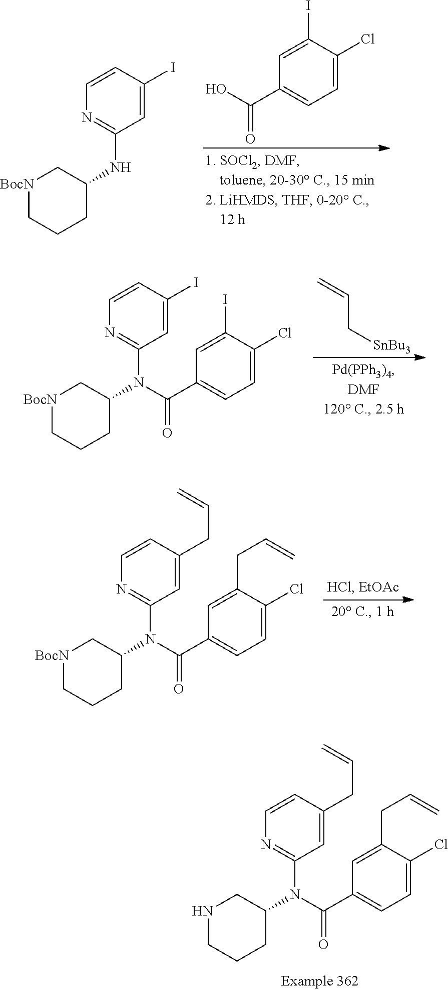

### Step 1:

To a solution of 4-chloro-3-iodobenzoic acid (420.27 mg, 1.49 mmol) in toluene (5 mL) were dropwise added DMF (9.06 mg, 123.99 μmol, 9.54 μL) and SOCl₂ (442.53 mg, 3.72 mmol, 270.17 μL) at 20° C. The mixture was stirred at 30° C. for 30 min, and then was concentrated. The residue was dissolved in THF (10 mL), and tert-butyl (3R)-3-[(4-iodo-2-pyridyl)amino]piperidine-1-carboxylate (0.5 g, 1.24 mmol) was added at 20° C. Then LiHMDS (1 M, 3.10 mL) was added at 0° C. dropwise under N₂ atmosphere, and the mixture was stirred at 20° C. for 2 h. The mixture was quenched by ice water (20 mL) and extracted with ethyl acetate (3×10 mL). The organic phases were combined, washed with saturated NaCl, dried with anhydrous sodium sulfate, and concentrated. The residue was purified by column chromatography (SiO₂, Petroleum ether/Ethyl acetate=100/5) to give tert-butyl(S)-3-(4-chloro-3-iodo-N-(4-iodopyridin-2-yl)benzamido)piperidine-1-carboxylate (0.05 g, 27%) as a yellow solid. LC-MS: [M+H⁺]=668.

### Step 2:

To a solution of tert-butyl (R)-3-(4-chloro-3-iodo-N-(4-iodopyridin-2-yl)benzamido)piperidine-1-carboxylate (500 mg, 673.95 μmol) in DMF (8 mL) were added allyltributylstannane (446.32 mg, 1.35 mmol, 413.64 μL) and Pd(PPh₃)₄ (77.88 mg, 67.40 μmol) at 20° C. The mixture was purged with N₂, and then the mixture was stirred at 130° C. for 2.5 h. The mixture was quenched with saturated aq. KF (50 mL) and extracted with EtOAc (90 mL). The organic phases were combined, washed with brine, dried with anhydrous sodium sulfate, and concentrated. The residue was purified by column chromatography (SiO₂, Petroleum ether/Ethyl acetate=100:3) to give tert-butyl (R)-3-(3-allyl-N-(4-allylpyridin-2-yl)-4-chlorobenzamido) piperidine-1-carboxylate (300 mg, 80.76%) as a yellow oil. LC-MS: [M+H⁺]=496.3.

### Step 3:

The title compound hydrochloride salt was obtained as an off-white solid by deprotection Method A used in Example 97. LC-MS: [M+H⁺]=396.3. ¹H NMR (400 MHZ, DMSO-d₆): δ 9.02-8.55 (m, 2H), 8.24-8.00 (m, 1H), 7.86-7.36 (m, 3H), 6.84-6.52 (m, 1H), 5.65 (br s, 2H), 5.28-4.99 (m, 2H), 4.97-4.76 (m, 2H), 4.41-4.23 (m, 1H), 3.53 (br s, 2H), 3.29-3.08 (m, 2H), 3.08-2.90 (m, 2H), 1.85-1.51 (m, 4H), 1.44-1.13 (m, 2H).

### Synthesis of 2-fluoro-4-(1-methyltriazol-4-yl)-N-[(3R)-3-piperidyl]-N-[2-(4-pyridyl)thieno[3,2-c]pyridin-4-yl]benzamide (Example 363)

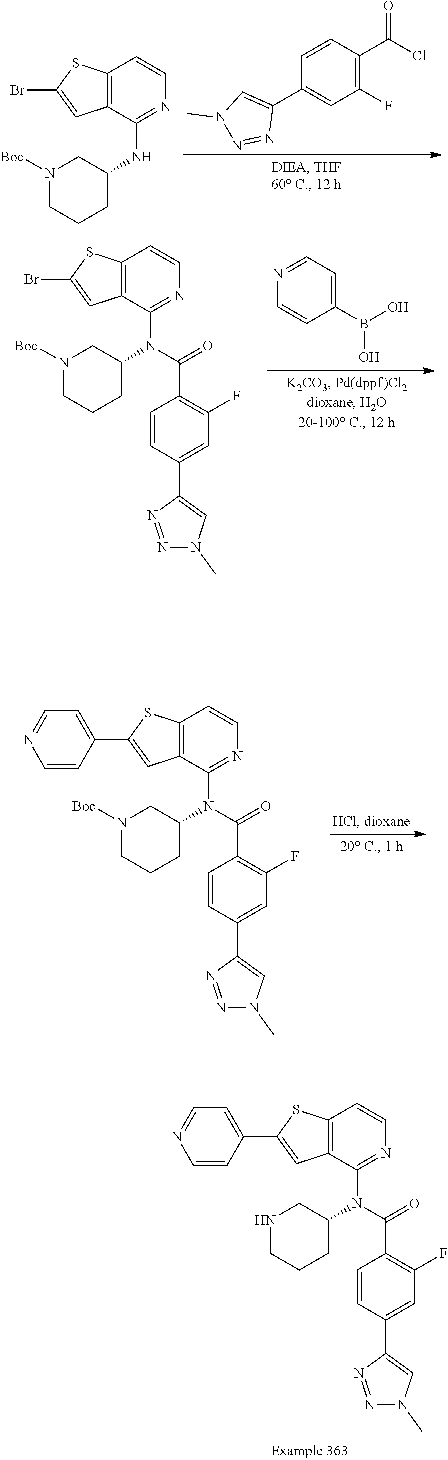

### Step 1:

To a solution of tert-butyl (3R)-3-[(2-bromothieno[3,2-c]pyridin-4-yl)amino]piperidine-1-carboxylate (5 g, 12.13 mmol) in THF (20 mL) were added DIEA (7.84 g, 60.63 mmol, 10.56 mL) and 2-fluoro-4-(1-methyltriazol-4-yl)benzoyl chloride (7.26 g, 30.31 mmol), and then the mixture was stirred at 60° C. for 4 h. The reaction was filtered, and the mixture was poured into water (50 mL) and extracted with ethyl acetate (3×50 mL). The organic phases were combined, washed with brine (2×50 mL), dried with anhydrous Na₂SO₄, and concentrated to give tert-butyl (3R)-3-[(2-bromothieno[3,2-c]pyridin-4-yl)-[2-fluoro-4-(1-methyltriazol-4-yl) benzoyl] amino] piperidine-1-carboxylate (7 g, 84%) as a gray solid. LC-MS: [M+H⁺]=615.2.

### Step 2:

A mixture of 4-pyridylboronic acid (53.92 mg, 438.66 μmol), tert-butyl (3R)-3-[(2-bromothieno[3,2-c]pyridin-4-yl)-[2-fluoro-4-(1-methyltriazol-4-yl)benzoyl]amino]piperidine-1-carboxylate (100 mg, 146.22 μmol), Pd(dppf)Cl₂ (10.70 mg, 14.62 μmol), K₂CO₃ (60.62 mg, 438.66 μmol) in dioxane (1.8 mL) and H₂O (0.2 mL) was degassed and purged with N₂ 3 times, and then the mixture was stirred at 100° C. for 12 h under N₂. The reaction mixture was filtered and concentrated to give tert-butyl (3R)-3-[[2-fluoro-4-(1-methyltriazol-4-yl)benzoyl]-[2-(4-pyridyl)thieno[3,2-c]pyridin-4-yl]amino]piperidine-1-carboxylate (200 mg) was obtained as a brown solid, which was used directly in the next step.

### Step 3:

The title compound hydrochloride salt was obtained as an off-white solid by deprotection Method A used in Example 97. LC-MS: [M+H⁺]=514.3. ¹H NMR (400 MHZ, DMSO-d₆): δ 9.53-9.06 (m, 2H), 8.93 (br s, 2H), 8.70-8.60 (m, 1H), 8.48-8.40 (m, 2H), 8.31 (br s, 2H), 8.12 (br d, J=5.3 Hz, 1H), 7.38-7.22 (m, 3H), 5.22-5.02 (m, 1H), 3.99 (s, 3H), 3.35 (br d, J=11.4 Hz, 1H), 3.18 (br d, J=10.1 Hz, 2H), 2.75-2.61 (m, 3H), 2.11-1.69 (m, 4H), 1.24-1.06 (m, 1H).

### Synthesis of 2-fluoro-N-[(3R)-3-piperidyl]-N-[2-(3-pyridyl)thieno[3,2-c]pyridin-4-yl]-4-(triazolo[4,5-b]pyridin-3-yl)benzamide (Example 364)

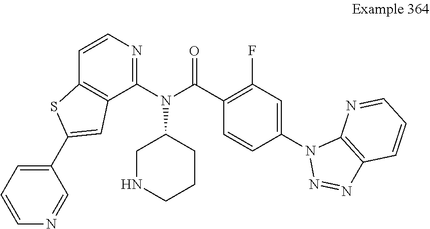 [SMILES: O=C(c1ccc(-n2nnc3cccnc32)cc1F)N(c1nccc2sc(-c3cccnc3)cc12)[C@@H]1CCCNC1]

Example 364 was prepared according to the procedure described for Example 161 by substituting 4-pyridylboronic acid with 3-pyridylboronic acid, provided the title compound hydrochloride salt as a light yellow solid. LC-MS: [M+H⁺]=551.4. ¹H NMR (400 MHZ, DMSO-d₆): δ 9.24-8.89 (m, 3H), 8.79 (br d, J=4.1 Hz, 1H), 8.71-8.64 (m, 2H), 8.43-8.29 (m, 3H), 8.09 (d, J=5.5 Hz, 1H), 7.99 (br d, J=10.6 Hz, 1H), 7.89 (br d, J=8.4 Hz, 1H), 7.67-7.51 (m, 3H), 5.18-4.91 (m, 1H), 3.78 (br s, 2H), 3.24-3.13 (m, 1H), 2.80-2.68 (m, 1H), 2.29-2.06 (m, 1H), 1.98-1.68 (m, 3H), 1.27-1.08 (m, 1H).

### Synthesis of 2-fluoro-N-[(3R)-3-piperidyl]-N-[2-(2-pyridyl)thieno[3,2-c]pyridin-4-yl]-4-(triazolo[4,5-b]pyridin-3-yl)benzamide (Example 365)

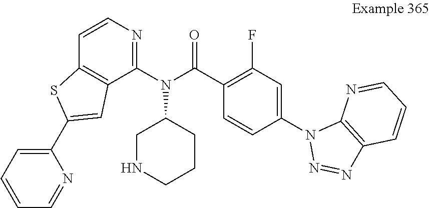 [SMILES: O=C(c1ccc(-n2nnc3cccnc32)cc1F)N(c1nccc2sc(-c3ccccn3)cc12)[C@@H]1CCCNC1]

Example 365 was prepared according to the procedure described for Example 161 by substituting 4-pyridylboronic acid with 2-pyridylboronic acid, provided the title compound hydrochloride salt as an off-white solid. LC-MS: [M+H⁺]=551.4. ¹H NMR (400 MHZ, DMSO-d₆): δ 9.32-8.88 (m, 2H), 8.78 (br d, J=3.8 Hz, 1H), 8.70-8.59 (m, 2H), 8.50-8.32 (m, 3H), 8.06-7.94 (m, 3H), 7.88 (br d, J=8.5 Hz, 1H), 7.64-7.49 (m, 2H), 7.49-7.41 (m, 1H), 5.22-4.93 (m, 1H), 3.75 (br d, J=11.6 Hz, 1H), 3.34 (br d, J=11.6 Hz, 1H), 3.19 (br d, J=10.1 Hz, 1H), 2.75-2.62 (m, 1H), 2.11 (br d, J=10.3 Hz, 1H), 1.97-1.69 (m, 3H), 1.27-1.07 (m, 1H).

### Synthesis of (R)-4-(3H-[1,2,3]triazolo[4,5-b]pyridin-3-yl)-2-fluoro-N-(6-(oxazol-2-yl)isoquinolin-1-yl)-N-(piperidin-3-yl)benzamide (Example 366)

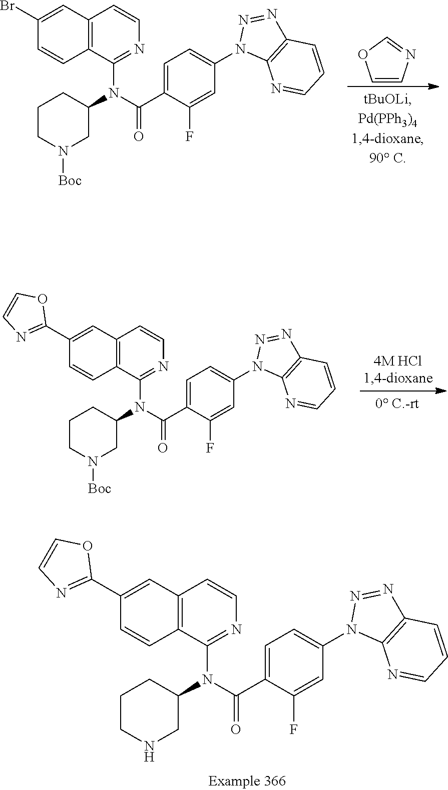 [SMILES: CC(C)(C)OC(=O)N1CCC[C@@H](N(C(=O)c2ccc(-n3nnc4cccnc43)cc2F)c2nccc3cc(Br)ccc23)C1 ; c1cocn1 ; CC(C)(C)OC(=O)N1CCC[C@@H](N(C(=O)c2ccc(-n3nnc4cccnc43)cc2F)c2nccc3cc(-c4ncco4)ccc23)C1 ; O=C(c1ccc(-n2nnc3cccnc32)cc1F)N(c1nccc2cc(-c3ncco3)ccc12)[C@@H]1CCCNC1]

### Step 1:

To a solution of tert-butyl (R)-3-(4-(3H-[1,2,3]triazolo[4,5-b]pyridin-3-yl)-N-(6-bromoisoquinolin-1-yl)-2-fluorobenzamido) piperidine-1-carboxylate (50 mg, 0.077 mmol) in 1,4-dioxane (1 mL) were added oxazole (8.01 mg, 0.116 mmol) and lithium 2-methylpropan-2-olate (18.57 mg, 0.232 mmol) at room temperature. The reaction mixture was purged with N₂ for 10 minutes. Then Pd(OAc)₂ (1.736 mg, 7.73 mmol) was added to the reaction mixture, and the reaction was stirred at 90° C. for 16 h. The reaction mixture was cooled to room temperature and diluted with ice cold water (5 mL) and EtOAc (20 mL). The organic layer was washed with brine, dried over Na₂SO₄, and concentrated. The obtained crude compound was purified by Combi-flash using eluting with 60% EtOAc in hexane and then was further purified by prep HPLC (Conditions: LUNA (250×21.2×5 μm), Mobile phase A: 0.1% formic acid in water (100%), Mobile phase B: 100% MeCN, Gradient: Time (min)/% B: 0/60, 10/80, Flow: 15 mL/min, Dilution: MeCN+water). Pure fractions were combined and concentrated to afford tert-butyl (R)-3-(4-(3H-[1,2,3]triazolo[4,5-b]pyridin-3-yl)-2-fluoro-N-(6-(oxazol-2-yl)isoquinolin-1-yl)benzamido)piperidine-1-carboxylate (8 mg, 2%) as an off white solid. LC-MS: [M+H⁺]=635.3.

### Step 2:

The title compound hydrochloride salt was obtained as an off-white solid by deprotection Method A used in Example 97. LC-MS: [M+H⁺]=535.2. ¹H NMR (CD₃OD, 400 MHZ): δ 8.72-8.71 (m, 1H), 8.51-8.45 (m, 2H), 8.36-8.34 (m, 1H), 8.28-8.25 (m, 1H), 8.22-8.18 (m, 1H), 8.14-8.03 (m, 2H), 7.93-7.90 (m, 1H), 7.83-7.78 (m, 2H), 7.52-7.48 (m, 1H), 7.35-7.31 (m, 1H), 5.32-5.28 (m, 1H), 4.22-3.96 (m, 1H), 3.74-3.68 (m, 1H), 2.91-2.89 (m, 1H), 2.06-1.91 (m, 3H), 1.43-1.28 (m, 1H), 0.96-0.89 (m, 1H).

### Synthesis of (R)-4-(3H-[1,2,3]triazolo[4,5-b]pyridin-3-yl)-2-fluoro-N-(piperidin-3-yl)-N-(6-(pyridin-3-yl)isoquinolin-1-yl)benzamide (Example 367)

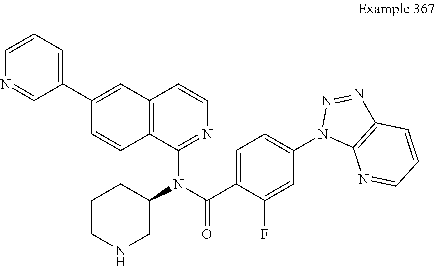 [SMILES: O=C(c1ccc(-n2nnc3cccnc32)cc1F)N(c1nccc2cc(-c3cccnc3)ccc12)[C@@H]1CCCNC1]

Example 367 was prepared according to the procedure described for Example 97 by substituting 1H-pyrazol-4-yl-boronic acid with pyridin-3-ylboronic acid, provided the title compound hydrochloride salt as an off-white solid. LC-MS: [M+H⁺]=545.25. ¹H NMR (CD₃OD, 400 MHZ): δ 9.36-9.33 (m, 1H), 9.08-9.04 (m, 1H), 8.93-8.90 (m, 1H), 8.73-8.72 (m, 1H), 8.61-8.60 (m, 1H), 8.49-8.47 (m, 1H), 8.43-8.39 (m, 2H), 8.25-8.19 (m, 2H), 8.03-8.00 (m, 1H), 7.95-7.90 (m, 2H), 7.53-7.50 (m, 1H), 7.38-7.34 (m, 1H), 5.37-5.31 (m, 1H), 4.01-3.99 (m, 1H), 3.78-3.72 (m, 1H), 3.35-3.29 (m, 1H), 2.92-2.85 (m, 1H), 2.12-2.07 (m, 1H), 1.97-1.93 (m, 2H), 1.39-1.28 (m, 1H).

### Synthesis of (R)-4-(3H-[1,2,3]triazolo[4,5-b]pyridin-3-yl)-2-fluoro-N-(piperidin-3-yl)-N-(6-(thiophen-2-yl)isoquinolin-1-yl)benzamide (Example 368)

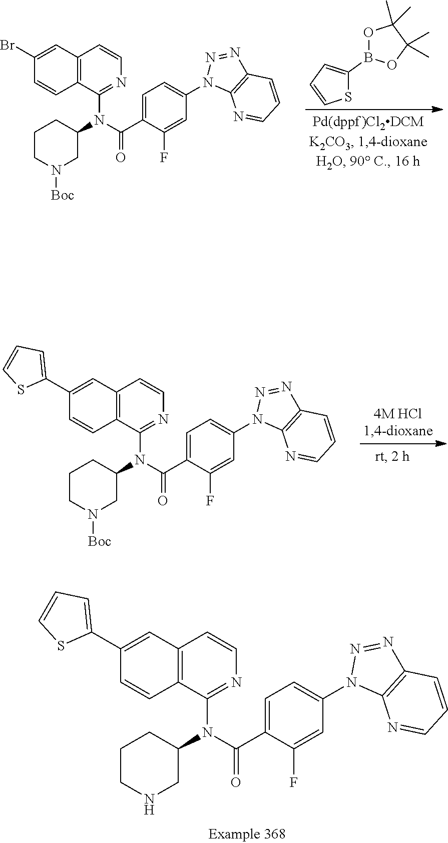

Example 368 was prepared according to the procedure described for Example 97 by substituting 1H-pyrazol-4-yl-boronic acid with 4,4,5,5-tetramethyl-2-(thiophen-2-yl)-1,3,2-dioxaborolane, provided the title compound hydrochloride salt as an off-white solid. LC-MS: [M+H⁺]=550.25. ¹H NMR (CD₃OD, 400 MHZ): δ 8.70-8.69 (m, 1H), 8.47-8.39 (m, 2H), 8.20-8.18 (m, 1H), 8.06-8.03 (m, 3H), 7.93-7.91 (m, 1H), 7.76 (d, J=5.6 Hz, 1H), 7.64-7.61 (m, 1H), 7.51-7.47 (m, 2H), 7.37-7.33 (m, 1H), 7.16-7.12 (m, 1H), 5.31-5.25 (m, 1H), 3.96-3.95 (m, 1H), 3.72-3.69 (m, 1H), 3.31-3.29 (m, 1H), 2.88-2.86 (m, 1H), 2.03-1.99 (m, 3H), 1.35-1.28 (m, 1H).

### Synthesis of (R)-1-(4-(3H-[1,2,3]triazolo[4,5-b]pyridin-3-yl)-2-fluoro-N-(piperidin-3-yl)benzamido)-N-methylisoquinoline-6-carboxamide (Example 369)

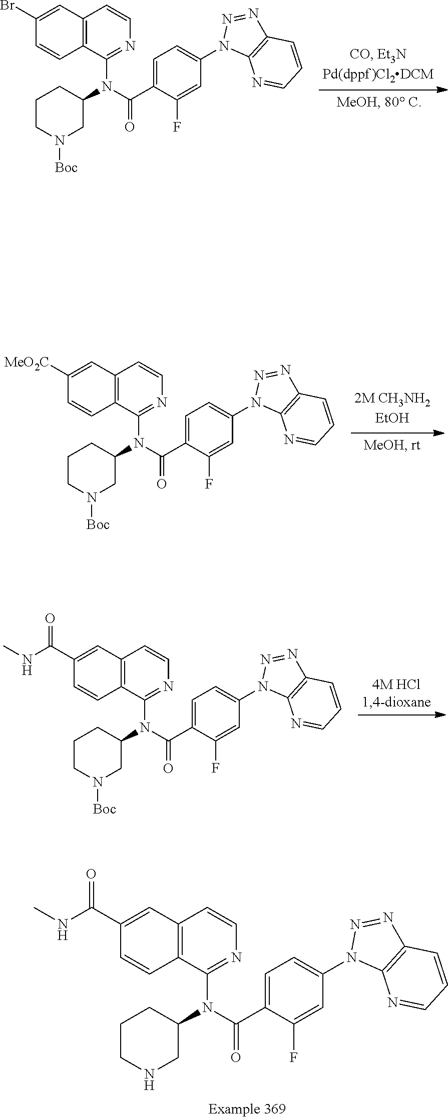 [SMILES: CC(C)(C)OC(=O)N1CCC[C@@H](N(C(=O)c2ccc(-n3nnc4cccnc43)cc2F)c2nccc3cc(Br)ccc23)C1 ; COC(=O)c1ccc2c(N(C(=O)c3ccc(-n4nnc5cccnc54)cc3F)[C@@H]3CCCN(C(=O)OC(C)(C)C)C3)nccc2c1 ; CNC(=O)c1ccc2c(N(C(=O)c3ccc(-n4nnc5cccnc54)cc3F)[C@@H]3CCCN(C(=O)OC(C)(C)C)C3)nccc2c1 ; CNC(=O)c1ccc2c(N(C(=O)c3ccc(-n4nnc5cccnc54)cc3F)[C@@H]3CCCNC3)nccc2c1]

To a solution of tert-butyl (R)-3-(4-(3H-[1,2,3]triazolo[4,5-b]pyridin-3-yl)-N-(6-bromoisoquinolin-1-yl)-2-fluorobenzamido) piperidine-1-carboxylate (200 mg, 0.309 mmol) in MeOH (10 mL) were added Et₃N (0.043 mL, 0.309 mmol) at room temperature, and the reaction mixture was degassed with nitrogen for 10 min and followed by addition of PdCl₂ (dppf)·DCM (253 mg, 0.309 mmol). The reaction mixture was filled with CO gas (100 psi) in a steel bomb and stirred at 90° C. for 4 h. The reaction was cooled to 25° C., diluted with water (20 mL), and extracted with EtOAc (2×40 mL). The combined organic layers were washed with brine, dried over Na₂SO₄, and concentrated. The residue was purified by Combi-flash using 40% EtOAc in hexane to afford tert-butyl (R)-3-(4-(3H-[1,2,3]triazolo[4,5-b]pyridin-3-yl)-N-(6-bromoisoquinolin-1-yl)-2 fluorobenzamido) piperidine-1-carboxylate (100 mg, 40%) as a light-brown solid. LC-MS: [M+H⁺]=626.30.

### Step 2:

To a solution of methyl (R)-1-(4-(3H-[1,2,3]triazolo[4,5-b]pyridin-3-yl)-N-(1-(tert-butoxycarbonyl)piperidin-3-yl)-2-fluorobenzamido) isoquinoline-6-carboxylate (50 mg, 0.080 mmol) in MeOH (2 mL) was added MeNH₂ (49.6 mg, 1.598 mmol) at room temperature, and the reaction was stirred for 16 h in a sealed tube. The reaction mixture was concentrated, and the residue was purified by prep-HPLC (Conditions: LUNA C-18 (250×21.2×5 μm), Mobile phase A: 0.1% formic acid in water (100%), Mobile phase B: 100% MeCN, Gradient: Time (min)/% B: 0/55, 10/75, Flow: 15 mL/min, Dilution: MeCN+H₂O+THF). The pure fraction was concentrated under reduced pressure to afford tert-butyl (R)-3-(4-(3H-[1,2,3]triazolo[4,5-b]pyridin-3-yl)-2-fluoro-N-(6-(methylcarbamoyl) isoquinolin-1-yl)benzamido)piperidine-1-carboxylate (20 mg, 44%) as an off-white solid. LC-MS: [M+H⁺]=625.25.

### Step 3:

The title compound hydrochloride salt was obtained as an off-white solid by deprotection Method A used in Example 97. LC-MS: [M+H⁺]=525.21. ¹H NMR (DMSO-d6, 400 MHz): δ 8.74 (d, J=4.0 Hz, 1H), 8.55 (d, J=5.6 Hz, 1H), 8.51-8.48 (m, 1H), 8.28-8.24 (m, 2H), 8.08-8.01 (m, 2H), 7.94-7.96 (m, 2H), 7.53-7.50 (m, 1H), 7.33-7.29 (m, 1H), 5.33-5.25 (m, 1H), 3.99-3.96 (m, 1H), 3.74-3.65 (m, 1H), 3.39-3.36 (m, 1H), 2.83 (s, 3H), 2.11-1.87 (m, 3H), 1.39-1.22 (m, 2H).

### Synthesis of (R)-2-fluoro-4-(1-methyl-1H-1,2,3-triazol-4-yl)-N-(piperidin-3-yl)-N-(6-(pyridin-3-yl) isoquinolin-1-yl)benzamide (Example 370)

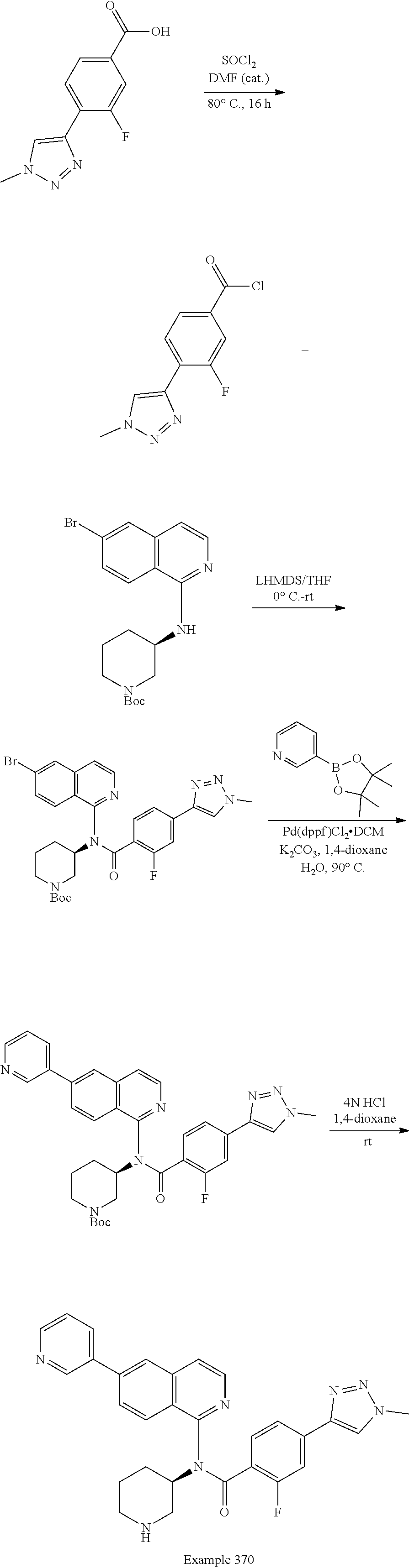

Example 370 was prepared according to the procedure described for Example 97 by substituting 1H-pyrazol-4-yl-boronic acid and 2-fluoro-4-(1-methyl-1H-1,2,3-triazol-4-yl)benzoic acid with pyridin-3-ylboronic acid and 3-fluoro-4-(1-methyl-1H-1,2,3-triazol-4-yl)benzoic acid, provided the title compound hydrochloride salt as an off-white solid. LC-MS: [M+H⁺]=508.2. ¹H NMR (CD₃OD, 400 MHZ): δ 9.47-9.3 (m, 1H), 9.13-9.05 (m, 2H), 8.93-8.91 (m, 1H), 8.60-8.52 (m, 1H), 8.42-8.31 (m, 2H), 8.25-8.15 (m, 3H), 7.93-7.91 (m, 1H), 7.28-7.10 (m, 2H), 5.34-5.28 (m, 1H), 4.05 (s, 3H), 3.97-3.94 (m, 1H), 3.75-3.69 (m, 1H), 3.37-3.30 (m, 1H), 2.90-2.83 (m, 1H), 2.03-1.91 (m, 3H), 1.33-1.28 (m, 1H).

### Synthesis of (R)-1-(4-(3H-[1,2,3]triazolo[4,5-b]pyridin-3-yl)-2-fluoro-N-(piperidin-3-yl)benzamido)-N-(2-hydroxyethyl) isoquinoline-6-carboxamide (Example 371)

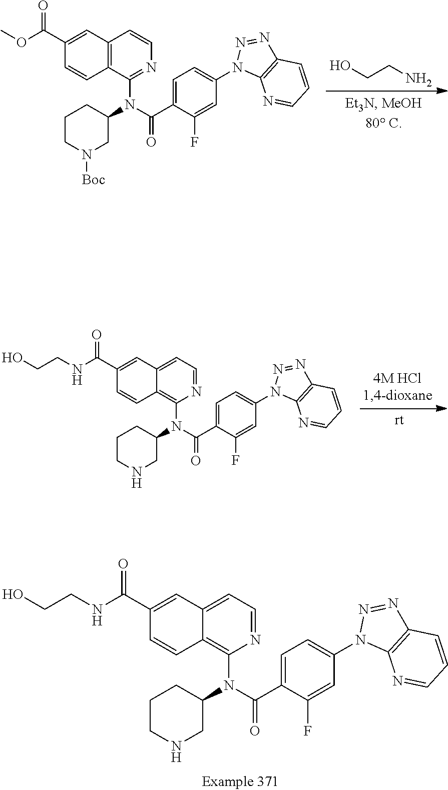

### Step 1:

To a solution of methyl (R)-1-(4-(3H-[1,2,3]triazolo[4,5-b]pyridin-3-yl)-N-(1-(tert-butoxycarbonyl)piperidin-3-yl)-2-fluorobenzamido) isoquinoline-6-carboxylate (50 mg, 0.080 mmol) in MeOH (2 mL) were added 2-aminoethan-1-ol (98 mg, 1.598 mmol) and Et₃N (0.011 mL, 0.080 mmol), and the reaction was stirred at 85° C. for 2 h. The reaction was concentrated, and the residue was purified by Combi-flash using 5% MeOH in DCM first and was further purified by prep HPLC (Conditions: Kinetex evo C-18 (250×21.2×5 μm), Mobile phase A: 0.1% formic acid in water (100%), Mobile phase B: 100% MeCN, Gradient: Time (min)/% B: 0/40, 10/70, Flow: 15 mL/min, Dilution: MeCN+H₂O+THF). The pure fractions were combined and concentrated to afford tert-butyl (R)-3-(4-(3H-[1,2,3]triazolo[4,5-b]pyridin-3-yl)-2-fluoro-N-(6-((2-hydroxyethyl) carbamoyl) isoquinolin-1-yl)benzamido-) piperidine-1-carboxylate (40 mg, 66%) as an off-white solid. LC-MS: [M+H⁺]=655.3.

### Step 2:

The title compound hydrochloride salt was obtained as an off-white solid by deprotection Method A used in Example 97. LC-MS: [M+H⁺]=555.20. ¹H NMR (DMSO-d₆, 400 MHZ): δ 8.73 (d, J=3.2 Hz, 1H), 8.55 (d, J=5.6 Hz, 1H), 8.52-8.48 (m, 1H), 8.32-8.26 (m, 2H), 8.11-8.09 (m, 1H), 8.04-8.01 (m, 1H), 7.91-7.86 (m, 2H), 7.53-7.50 (m, 1H), 7.33-7.29 (m, 1H), 5.33-5.26 (m, 1H), 3.99-3.96 (m, 1H), 3.72-3.67 (m, 3H), 3.50-3.49 (m, 2H), 3.36-3.33 (m, 1H), 2.88-2.85 (m, 1H), 2.04-1.91 (m, 3H), 1.35-1.22 (m, 1H).

### Synthesis of (R)-1-(4-(3H-[1,2,3]triazolo[4,5-b]pyridin-3-yl)-2-fluoro-N-(piperidin-3-yl)benzamido)-N-isopropylisoquinoline-6-carboxamide (Example 372)

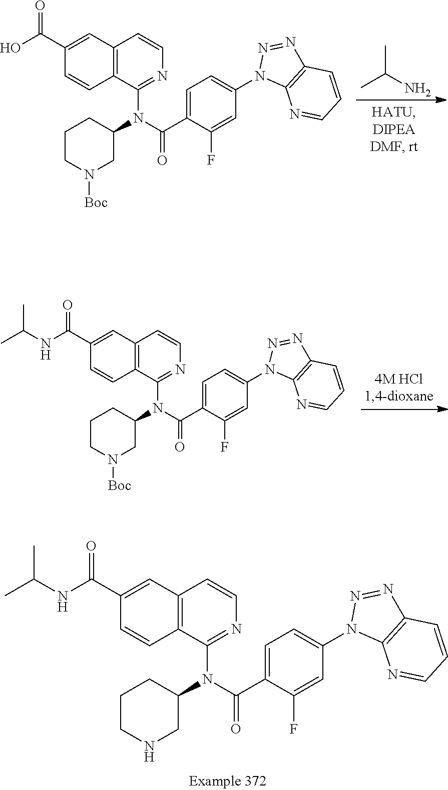

### Step 1:

To a solution of (R)-1-(4-(3H-[1,2,3]triazolo[4,5-b]pyridin-3-yl)-N-(1-(tert-butoxycarbonyl)piperidin-3-yl)-2-fluorobenzamido) isoquinoline-6-carboxylic acid (100 mg, 0.163 mmol) in DMF (2 mL) at 0° C. were added DIPEA (0.086 ml, 0.490 mmol), HATU (93 mg, 0.245 mmol), and propan-2-amine (14.50 mg, 0.245 mmol). The reaction was stirred at room temperature for 16 h. The reaction mixture was poured into ice cold water, and the precipitate was filtered, dried, and was purified by prep HPLC (Conditions: X-SELECT CSH, Mobile phase A: 0.1% formic acid in water (100%), Mobile phase B: 100% MeCN, Gradient: Time (min)/% B: 0/55, 10/80, Flow: 15 mL/min, Dilution: MeCN+H₂O+THF). The pure fractions were concentrated to afford tert-butyl (R)-3-(4-(3H-[1,2,3]triazolo[4,5-b]pyridin-3-yl)-2-fluoro-N-(6-(isopropylcarbamoyl) isoquinolin-1-yl)benzamido)piperidine-1-carboxylate (40 mg, 0.04 mmol) as a white solid. LC-MS: [M+H⁺]=653.3.

### Step 2:

To a solution of tert-butyl (R)-3-(4-(3H-[1,2,3]triazolo[4,5-b]pyridin-3-yl)-2-fluoro-N-(6-(isopropylcarbamoyl) isoquinolin-1-yl)benzamido)piperidine-1-carboxylate (40 mg, 0.061 mmol) at 0° C. was added 4M HCl in 1,4-dioxane (2 mL). The reaction was stirred at room temperature for 2 h. The reaction mixture was concentrated, and the residue was purified by prep HPLC (Conditions: Kinetex evo C-18 (250×21.2×5 μm), Mobile phase A: 0.1% formic acid in water (100%), Mobile phase B: 100% MeCN, Gradient: Time (min)/% B: 0/55, 10/80, Flow: 15 mL/min, Dilution: MeCN+H₂O+THF). The pure fractions were combined and concentrated, and the residue was again purified by prep HPLC (Conditions: Kinetex evo C-18 (250×21.2×5 μm), Mobile phase A: 0.1% formic acid in water (100%), Mobile phase B: 100% MeCN, Gradient: Time (min)/% B: 0/55, 10/80, Flow: 15 mL/min, Dilution: MeCN+H₂O+THF). The pure fractions were combined and concentrated to afford (R)-1-(4-(3H-[1,2,3]triazolo[4,5-b]pyridin-3-yl)-2-fluoro-N-(piperidin-3-yl)benzamido)-N-isopropyl-isoquinoline-6-carboxamide formic acid salt (12 mg) as an off-white solid. LC-MS: [M+H⁺]=553.25. ¹H NMR (DMSO-d₆, 400 MHz): δ 8.73 (d, J=3.6 Hz, 1H), 8.54-8.48 (m, 2H), 8.27-8.22 (m, 2H), 8.10-8.00 (m, 2H), 7.92-7.88 (m, 1H), 7.85-7.83 (m, 1H), 7.53-7.50 (m, 1H), 7.38-7.28 (m, 1H), 5.22-5.16 (m, 1H), 4.21-4.14 (m, 1H), 3.88-3.85 (m, 1H), 3.59-3.47 (m, 1H), 3.21-3.13 (m, 1H), 2.78-2.62 (m, 1H), 2.01-1.98 (m, 1H), 1.88-1.78 (m, 2H), 1.32-1.25 (m, 1H), 1.23-1.20 (m, 6H).

### Synthesis of (R)-1-(4-(3H-[1,2,3]triazolo[4,5-b]pyridin-3-yl)-2-fluoro-N-(piperidin-3-yl)benzamido)-N-benzylisoquinoline-6-carboxamide (Example 373)

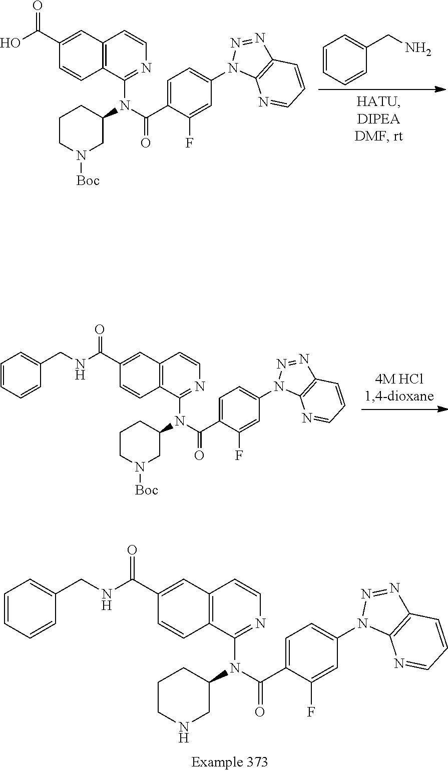

Example 373 was prepared according to the procedure for Example 372 by substituting propan-2-amine with benzyl amine, providing the title compound as a white solid. LC-MS: [M+H⁺]=601.25. ¹H NMR (DMSO-d₆, 400 MHZ): δ 8.72 (d, J=4.4 Hz, 1H), 8.55-8.54 (m, 1H), 8.51-8.48 (m, 1H), 8.31-8.27 (m, 2H), 8.16-8.10 (m, 1H), 8.04-8.01 (m, 1H), 7.93-7.86 (m, 2H), 7.53-7.50 (m, 1H), 7.38-7.22 (m, 6H), 5.32-5.29 (m, 1H), 4.56 (s, 2H), 3.99-3.96 (m, 1H), 3.74-3.68 (m, 2H), 2.88-2.82 (m, 1H), 2.04-1.90 (m, 3H), 1.32-1.28 (m, 1H).

### Synthesis of 4-[4-[[2-fluoro-4-(triazolo[4,5-b]pyridin-3-yl)benzoyl]-[(3R)-3-piperidyl]amino] thieno[3,2-c]pyridin-2-yl]-N-methyl-pyridine-2-carboxamide (Example 374)

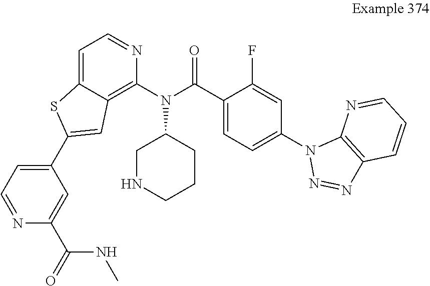 [SMILES: CNC(=O)c1cc(-c2cc3c(N(C(=O)c4ccc(-n5nnc6cccnc65)cc4F)[C@@H]4CCCNC4)nccc3s2)ccn1]

Example 374 was prepared according to the procedure described for Example 161 by substituting 4-pyridylboronic acid with N-methyl-4-(4,4,5,5-tetramethyl-1,3,2-dioxaborolan-2-yl) picolinamide, providing the title compound (21%) as a white solid. LC-MS: [M+H⁺]=608.4.

1H NMR (400 MHZ, DMSO-d₆): δ 9.36-9.17 (m, 1H), 9.14-8.94 (m, 1H), 8.93-8.86 (m, 1H), 8.83-8.73 (m, 2H), 8.71-8.59 (m, 2H), 8.53-8.38 (m, 2H), 8.19-8.05 (m, 2H), 7.99 (br d, J=10.1 Hz, 1H), 7.93-7.82 (m, 1H), 7.65-7.46 (m, 2H), 5.30-4.97 (m, 1H), 3.47-3.10 (m, 2H), 2.88 (d, J=4.9 Hz, 3H), 2.80-2.70 (m, 1H), 2.26-2.05 (m, 2H), 1.95-1.69 (m, 2H), 1.28-1.02 (m, 1H).

### Synthesis of methyl 4-[4-[[2-fluoro-4-(triazolo[4,5-b]pyridin-3-yl)benzoyl]-[(3R)-3-piperidyl]amino]thieno[3,2-c]pyridin-2-yl]benzoate (Example 375)

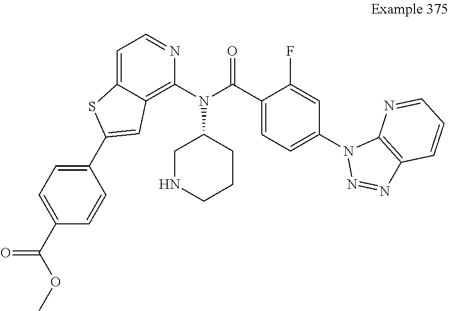 [SMILES: COC(=O)c1ccc(-c2cc3c(N(C(=O)c4ccc(-n5nnc6cccnc65)cc4F)[C@@H]4CCCNC4)nccc3s2)cc1]

Example 375 was prepared according to the procedure described for Example 161 by substituting 4-pyridylboronic acid with methyl 4-(4,4,5,5-tetramethyl-1,3,2-dioxaborolan-2-yl)benzoate, provided the title compound hydrochloride salt as a white solid. LC-MS: [M+H⁺]=608.4. ¹H NMR (400 MHz, DMSO-d₆): δ 9.19-8.84 (m, 2H), 8.83-8.73 (m, 1H), 8.67 (br d, J=8.4 Hz, 1H), 8.46-8.35 (m, 1H), 8.35-8.21 (m, 1H), 8.18-7.96 (m, 6H), 7.93-7.84 (m, 1H), 7.63-7.49 (m, 2H), 5.17-4.94 (m, 1H), 3.94-3.87 (m, 3H), 3.81-3.71 (m, 1H), 3.40-3.30 (m, 1H), 3.20 (br d, J=12.3 Hz, 1H), 2.82-2.70 (m, 1H), 2.18-2.04 (m, 1H), 1.94-1.70 (m, 2H), 1.26-1.08 (m, 1H).

### Synthesis of 2-fluoro-N-[2-(3-hydroxy-3-methyl-but-1-ynyl)thieno[3,2-c]pyridin-4-yl]-N-[(3R)-3-piperidyl]-4-(triazolo[4,5-b]pyridin-3-yl)benzamide (Example 376)

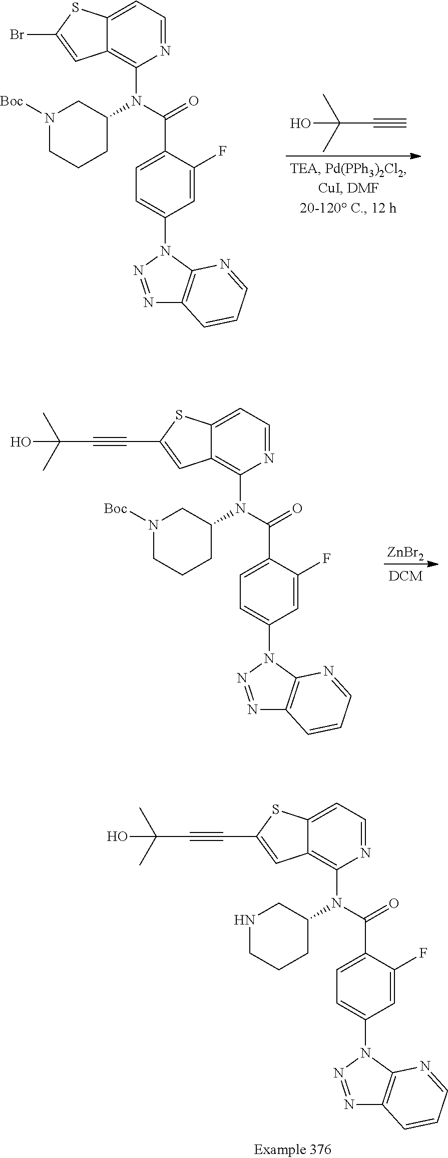

To a solution of tert-butyl (3R)-3-[(2-bromothieno[3,2-c]pyridin-4-yl)-[2-fluoro-4-(triazolo[4,5-b]pyridin-3-yl)benzoyl]amino]piperidine-1-carboxylate (200 mg, 306.50 μmol) in DMA (3 mL) at 20° C. were added 2-methylbut-3-yn-2-ol (257.81 mg, 3.06 mmol, 299.43 μL), TEA (93.04 mg, 919.49 μmol, 127.98 μL), Pd(PPh₃)₂Cl₂ (21.51 mg, 30.65 μmol) and CuI (5.84 mg, 30.65 μmol). The mixture was stirred at 120° C. for 12 h before it was poured into water (50 mL) and extracted with EtOAc (3×50 mL). The organic phases were combined, washed with brine (20 mL), dried with anhydrous sodium sulfate, and concentrated. The residue was purified by prep-TLC (Petroleum ether:Ethyl acetate=0:1) to give tert-butyl (R)-3-(4-(3H-[1,2,3]triazolo[4,5-b]pyridin-3-yl)-2-fluoro-N-(2-(3-hydroxy-3-methylbut-1-yn-1-yl)thieno[3,2-c]pyridin-4-yl)benzamido)piperidine-1-carboxylate (100 mg, 49%) as a white solid. LC-MS: [M+H⁺]=656.3.

### Step 2:

To a solution of tert-butyl (3R)-3-[[2-fluoro-4-(triazolo[4,5-b]pyridin-3-yl)benzoyl]-[2-(3-hydroxy-3-methyl-but-1-ynyl)thieno[3,2-c]pyridin-4-yl]amino]piperidine-1-carboxylate (40 mg, 61.00 μmol) in DCM (1 mL) at 20° C. was added ZnBr₂ (68.69 mg, 305.00 μmol, 15.26 μL), and then the reaction was stirred at 20° C. for 12 h. The mixture was concentrated, and the residue was purified by prep-HPLC under neutral system (column: Waters Xbridge C18 150*50 mm*10 μm; mobile phase: [H₂O (10 mM NH₄HCO₃)-MeCN]; gradient: 25%-60% B over 8.0 min) and lyophilized to give (R)-4-(3H-[1,2,3]triazolo[4,5-b]pyridin-3-yl)-2-fluoro-N-(2-(3-hydroxy-3-methylbut-1-yn-1-yl)thieno[3,2-c]pyridin-4-yl)-N-(piperidin-3-yl)benzamide (6.6 mg, 18%) as a white solid. LC-MS: [M+H⁺]=556.1. ¹H NMR (400 MHZ, DMSO-d₆): δ 9.07-8.61 (m, 2H), 8.55-8.07 (m, 2H), 8.06-7.81 (m, 2H), 7.76-7.54 (m, 2H), 7.49-7.29 (m, 1H), 5.70 (s, 1H), 4.79-4.32 (m, 1H), 3.20-3.02 (m, 1H), 2.85-2.70 (m, 1H), 2.23-2.07 (m, 2H), 1.91 (br s, 1H), 1.67-1.36 (m, 9H), 1.11-0.75 (m, 1H).

### Synthesis of (R)-4-(3H-[1,2,3]triazolo[4,5-b]pyridin-3-yl)-2-fluoro-N-(2-(4-hydroxyphenyl)thieno[3,2-c]pyridin-4-yl)-N-(piperidin-3-yl)benzamide (Example 377)

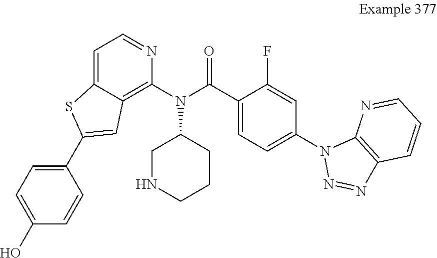 [SMILES: O=C(c1ccc(-n2nnc3cccnc32)cc1F)N(c1nccc2sc(-c3ccc(O)cc3)cc12)[C@@H]1CCCNC1]

Example 377 was prepared according to the procedure described for Example 161 by substituting 4-pyridylboronic acid with 4-(4,4,5,5-tetramethyl-1,3,2-dioxaborolan-2-yl) phenol, providing the title compound as a white solid. LC-MS: [M+H⁺]=566.4. ¹H NMR (400 MHZ, DMSO-d₆): δ 8.81-8.80 (m, 1H), 8.68-8.66 (m, 1H), 8.30-2.82 (m, 1H), 7.91-7.83 (m, 2H), 7.74 (s, 1H), 7.70-7.68 (m, 2H), 7.5-7.61 (m, 1H), 7.59-7.45 (M, 1H), 6.97-6.89 (m, 2H), 4.70-4.64 (m, 1H), 2.86-2.76 (m, 2H), 2.26-2.10 (m, 2H), 2.00-1.92 (m, 1H), 1.85-1.43 (m, 3H), 1.04-0.98 (m, 1H).

### Synthesis of 2-fluoro-N-[2-(4-methoxyphenyl)thieno[3,2-c]pyridin-4-yl]-N-[(3R)-3-piperidyl]-4-(triazolo[4,5-b]pyridin-3-yl)benzamide (Example 378)

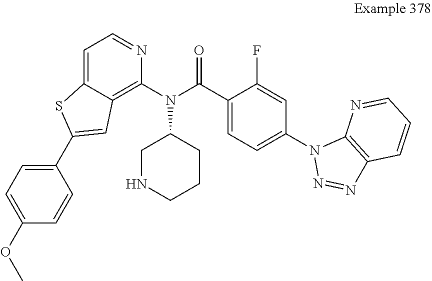 [SMILES: COc1ccc(-c2cc3c(N(C(=O)c4ccc(-n5nnc6cccnc65)cc4F)[C@@H]4CCCNC4)nccc3s2)cc1]

Example 378 was prepared according to the procedure described for Example 161 by substituting 4-pyridylboronic acid with 2-(4-methoxyphenyl)-4,4,5-trimethyl-1,3,2-dioxaborolane, provided the title compound hydrochloride salt as a white solid. LC-MS: [M+H⁺]=580.4. ¹H NMR (400 MHZ, DMSO-d₆): δ 9.29-8.88 (m, 2H), 8.80 (d, J=3.9 Hz, 1H), 8.67 (d, J=8.1 Hz, 1H), 8.37-8.17 (m, 1H), 8.09-7.66 (m, 6H), 7.63-7.40 (m, 2H), 7.09 (br d, J=8.5 Hz, 2H), 5.26-4.90 (m, 1H), 3.89-3.81 (m, 3H), 3.80-3.67 (m, 1H), 3.36-3.09 (m, 2H), 2.84-2.69 (m, 1H), 2.30-1.98 (m, 1H), 1.96-1.67 (m, 2H), 1.27-1.00 (m, 1H).

### Synthesis of 4-[4-[[2-fluoro-4-(triazolo[4,5-b]pyridin-3-yl)benzoyl]-[(3R)-3-piperidyl]amino] thieno[3,2-c]pyridin-2-yl]benzoic acid (Example 379)

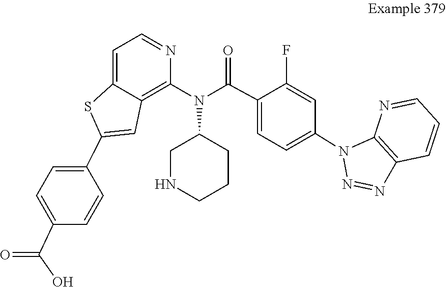 [SMILES: O=C(O)c1ccc(-c2cc3c(N(C(=O)c4ccc(-n5nnc6cccnc65)cc4F)[C@@H]4CCCNC4)nccc3s2)cc1]

Example 379 was prepared according to the procedure described for Example 161 by substituting 4-pyridylboronic acid with 4-(4,4,5,5-tetramethyl-1,3,2-dioxaborolan-2-yl)benzoic acid, providing the title compound as a white solid. LC-MS: [M+H⁺]=594.4. ¹H NMR (400 MHZ, DMSO-d6): δ 13.36-13.04 (m, 1H), 9.05-8.75 (m, 3H), 8.67 (d, J=8.3 Hz, 1H), 8.46-8.20 (m, 2H), 8.09-7.98 (m, 5H), 7.89 (br d, J=8.6 Hz, 1H), 7.62-7.51 (m, 2H), 5.25-4.90 (m, 1H), 3.92-3.68 (m, 1H), 3.21 (br d, J=12.4 Hz, 1H), 2.72 (br d, J=9.6 Hz, 1H), 2.24-2.07 (m, 1H), 1.97-1.63 (m, 3H), 1.27-1.04 (m, 1H).

### Synthesis of 2-fluoro-N-[2-[4-(methylcarbamoyl)phenyl]thieno[3,2-c]pyridin-4-yl]-N-[(3R)-3-piperidyl]-4-(triazolo[4,5-b]pyridin-3-yl)benzamide (Example 380)

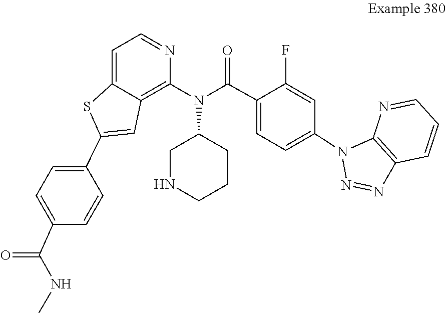 [SMILES: CNC(=O)c1ccc(-c2cc3c(N(C(=O)c4ccc(-n5nnc6cccnc65)cc4F)[C@@H]4CCCNC4)nccc3s2)cc1]

Example 380 was prepared according to the procedure described for Example 161 by substituting 4-pyridylboronic acid with [4-(methylcarbamoyl)phenyl]boronic acid, provided the title compound hydrochloride salt as a white solid. LC-MS: [M+H⁺]=607.4. ¹H NMR (400 MHZ, DMSO-d₆) δ 8.79 (br d, J=3.5 Hz, 1H), 8.66 (br d, J=8.0 Hz, 1H), 8.57 (br d, J=4.4 Hz, 1H), 8.35 (br s, 1H), 8.15 (s, 1H), 8.03-7.93 (m, 6H), 7.84 (br d, J=8.3 Hz, 1H), 7.61-7.45 (m, 2H), 4.77-4.60 (m, 1H), 2.93-2.85 (m, 1H), 2.82 (d, J=4.5 Hz, 3H), 2.78 (br d, J=10.4 Hz, 1H), 2.22-2.11 (m, 2H), 2.03-1.95 (m, 1H), 1.79-1.60 (m, 1H), 1.59-1.40 (m, 2H), 1.08-0.87 (m, 1H).

### Synthesis of 2-fluoro-N-[2-(3-hydroxyphenyl)thieno[3,2-c]pyridin-4-yl]-N-[(3R)-3-piperidyl]-4-(triazolo[4,5-b]pyridin-3-yl)benzamide (Example 381)

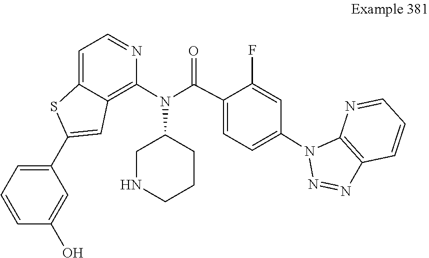 [SMILES: O=C(c1ccc(-n2nnc3cccnc32)cc1F)N(c1nccc2sc(-c3cccc(O)c3)cc12)[C@@H]1CCCNC1]

Example 381 was prepared according to the procedure described for Example 161 by substituting 4-pyridylboronic acid with (3-hydroxyphenyl) boronic acid, provided the title compound hydrochloride salt as an off white solid. LC-MS: [M+H⁺]=566.4. ¹H NMR (400 MHz, DMSO-d₆): δ 0.91-1.08 (m, 1H), 1.38-1.75 (m, 3H), 1.94-2.02 (m, 1H), 2.14-2.26 (m, 2H), 2.72-2.80 (m, 1H), 4.54-4.75 (m, 1H), 6.86 (br d, J=7.63 Hz, 1H), 7.22 (br s, 1H), 7.27-7.36 (m, 2H), 7.39-7.52 (m, 1H), 7.59 (br dd, J=8.00, 4.25 Hz, 1H), 7.81-8.03 (m, 4H), 8.30-8.37 (m, 1H), 8.59-8.72 (m, 1H), 8.80 (br d, J=3.63 Hz, 1H), 9.70-9.87 (m, 1H).

### Synthesis of N-(2-ethynylthieno[3,2-c]pyridin-4-yl)-2-fluoro-N-[(3R)-3-piperidyl]-4-(triazolo[4,5-b]pyridin-3-yl)benzamide (Example 382)

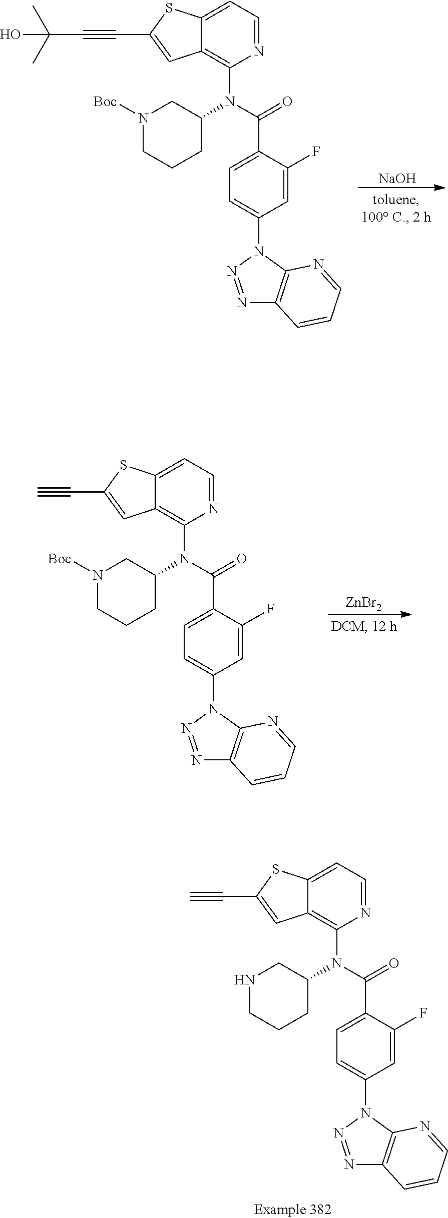 [SMILES: CC(C)(O)C#Cc1cc2c(N(C(=O)c3ccc(-n4nnc5cccnc54)cc3F)[C@@H]3CCCN(C(=O)OC(C)(C)C)C3)nccc2s1 ; "NaOH" ; C#Cc1cc2c(N(C(=O)c3ccc(-n4nnc5cccnc54)cc3F)[C@@H]3CCCN(C(=O)OC(C)(C)C)C3)nccc2s1 ; "ZnBr2" ; C#Cc1cc2c(N(C(=O)c3ccc(-n4nnc5cccnc54)cc3F)[C@@H]3CCCNC3)nccc2s1]

### Step 1:

To a solution of tert-butyl (3R)-3-[[2-fluoro-4-(triazolo[4,5-b]pyridin-3-yl)benzoyl]-[2-(3-hydroxy-3-methyl-but-1-ynyl)thieno[3,2-c]pyridin-4-yl]amino]piperidine-1-carboxylate (50 mg, 76.25 μmol) in toluene (2 mL) at 20° C. was added NaOH (30.50 mg, 762.50 μmol). The mixture was stirred at 100° C. for 2 h, after which it was poured into water (10 mL) and extracted with ethyl acetate (3×10 mL). The organic phases were combined, washed with brine (5 ml), dried with anhydrous sodium sulfate, and concentrated to give tert-butyl (3R)-3-[(2-ethynylthieno[3,2-c]pyridin-4-yl)-[2-fluoro-4-(triazolo[4,5-b]pyridin-3-yl)benzoyl]amino] piperidine-1-carboxylate (40 mg, 87%) as a yellow solid.

### Step 2:

To a solution of tert-butyl (3R)-3-[(2-ethynylthieno[3,2-c]pyridin-4-yl)-[2-fluoro-4-(triazolo [4,5-b]pyridin-3-yl)benzoyl]amino]piperidine-1-carboxylate (0.05 g, 83.66 μmol) in DCM (2 mL) at 20° C. was added ZnBr₂ (94.20 mg, 418.30 μmol, 20.93 μL). The mixture was stirred at 20° C. for 5 h, after which it was concentrated. The residue was purified by prep-HPLC under neutral system (column: Waters Xbridge BEH C18 100*30 mm*10 μm; mobile phase: [H₂O (10 mM NH₄HCO₃)-MeCN]; gradient: 30%-60% B over 8.0 min) and lyophilized to give N-(2-ethynylthieno[3,2-c]pyridin-4-yl)-2-fluoro-N-[(3R)-3-piperidyl]-4-(triazolo[4,5-b]pyridin-3-yl)benzamide (7 mg, 15%) as a white solid. LC-MS: [M+H⁺]=498.3. ¹H NMR (400 MHZ, DMSO-d₆): δ 9.05-8.61 (m, 2H), 8.56-8.30 (m, 1H), 8.07-7.77 (m, 3H), 7.71-7.51 (m, 2H), 7.49-7.33 (m, 1H), 5.10-4.86 (m, 1H), 4.79-4.46 (m, 1H), 2.93-2.71 (m, 2H), 2.27-2.09 (m, 2H), 2.00-1.85 (m, 1H), 1.77-1.40 (m, 3H), 1.07-0.72 (m, 1H).

### Synthesis of N-[2-(2-amino-4-pyridyl)thieno[3,2-c]pyridin-4-yl]-2-fluoro-N-[(3R)-3-piperidyl]-4-(triazolo[4,5-b]pyridin-3-yl)benzamide (Example 383)

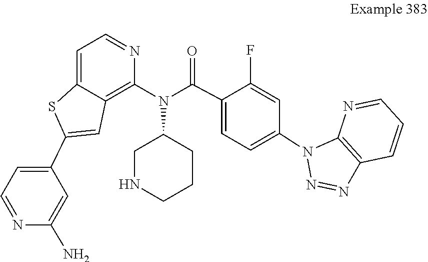 [SMILES: Nc1cc(-c2cc3c(N(C(=O)c4ccc(-n5nnc6cccnc65)cc4F)[C@@H]4CCCNC4)nccc3s2)ccn1]

Example 383 was prepared according to the procedure described for Example 161 by substituting 4-pyridylboronic acid with (6-aminopyridin-3-yl)boronic acid, provided the title compound hydrochloride salt (80%) as a yellow solid. LC-MS: [M+H⁺]=566.4. ¹H NMR (400 MHZ, CD₃OD): δ 1.43 (br d, J=4.75 Hz, 1H), 1.96 (br s, 2H), 2.12 (br d, J=9.88 Hz, 1H), 2.91 (br s, 1H), 3.38 (br d, J=12.13 Hz, 1H), 3.73 (br t, J=10.88 Hz, 1H), 3.88-3.99 (m, 1H), 5.30 (br s, 1H), 7.11-7.37 (m, 1H), 7.38-7.64 (m, 3H), 7.86-8.11 (m, 4H), 8.22-8.32 (m, 1H), 8.45-8.64 (m, 2H), 8.69-8.79 (m, 1H).

### Synthesis of 2-fluoro-N-[(3R)-3-piperidyl]-N-[2-(4-pyridyl)thieno[2,3-c]pyridin-7-yl]-4-(triazolo[4,5-b]pyridin-3-yl)benzamide (Example 384)

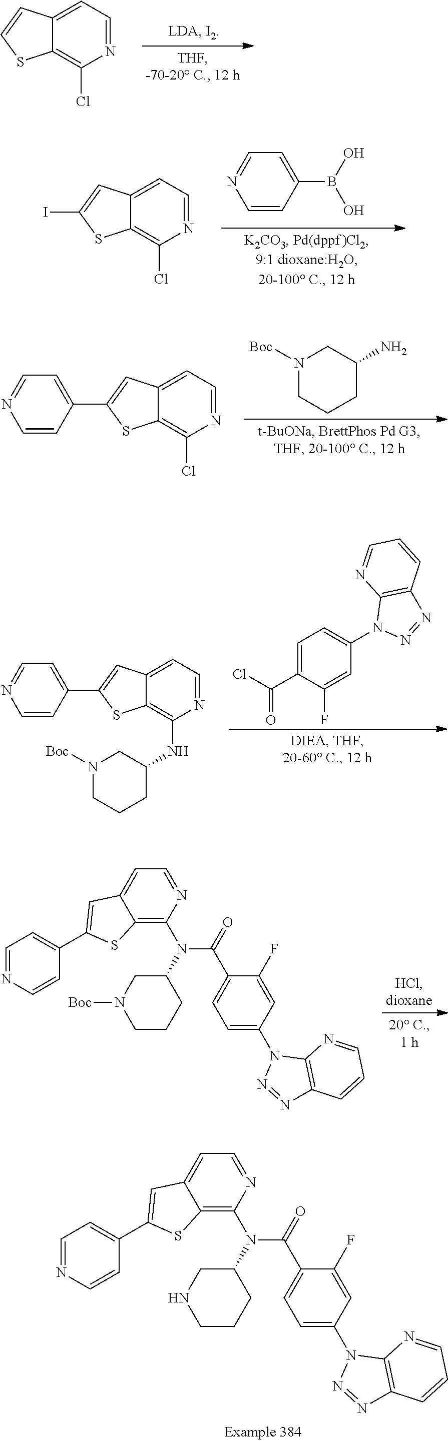

### Step 1:

To a solution of 7-chlorothieno[2,3-c]pyridine (0.8 g, 4.72 mmol) in THF (40 mL) at −70° C. was added LDA (2 M, 2.83 mL) dropwise under N₂. The reaction mixture was stirred at −70° C. for 1 h. Then I₂ (1.80 g, 7.07 mmol, 1.42 mL) in THF (10 mL) was added to the reaction dropwise at −70° C. under N₂. The mixture was stirred at 20° C. for 12 h. The reaction mixture was quenched with NH₄Cl (50 mL), and the mixture was concentrated. The reaction mixture was filtered, and the filtrate was concentrated to give 7-chloro-2-iodo-thieno[2,3-c]pyridine (1.6 g, 82%) as a light yellow solid.

### Step 2:

To a solution of 7-chloro-2-iodo-thieno[2,3-c]pyridine (400 mg, 1.22 mmol) and 4-pyridylboronic acid (179.68 mg, 1.46 mmol) in dioxane (14.4 mL) and H₂O (1.6 mL) at 20° C. were added K₂CO₃ (336.71 mg, 2.44 mmol) and Pd(dppf)Cl₂ (89.13 mg, 121.82 μmol) under N₂. The mixture was stirred at 100° C. for 12 h. The reaction mixture was poured into water (50 mL) and extracted with EtOAc (3×50 mL). The combined organic phase was washed with brine (100 mL), dried with Na₂SO₄, and concentrated. The residue was purified by column chromatography (SiO₂, Petroleum ether/EtOAc=60/40 to 50/50), and the product was concentrated to give 7-chloro-2-(4-pyridyl)thieno[2,3-c]pyridine (0.18 g, 53%) as a solid.

### Step 3:

To a solution of 7-chloro-2-(4-pyridyl)thieno[2,3-c]pyridine (200 mg, 729.59 μmol) and tert-butyl (3R)-3-aminopiperidine-1-carboxylate (292.24 mg, 1.46 mmol) in THF (20 mL) were added t-BuONa (210.35 mg, 2.19 mmol) and BrettPhos Pd G3 (198.41 mg, 218.88 μmol) in order at 20° C. under N₂. The mixture was stirred at 80° C. for 12 h before it was concentrated. The residue was poured into water (20 mL) and extracted with ethyl acetate (3×20 mL). The combined organic phase was washed with brine (50 mL), dried with anhydrous Na₂SO₄, and concentrated. The residue was purified by column chromatography (SiO₂, Petroleum ether/Ethyl acetate=50/50 to 40/60), and the product was concentrated to give tert-butyl (3R)-3-[[2-(4-pyridyl)thieno[2,3-c]pyridin-7-yl]amino]piperidine-1-carboxylate (0.3 g, 72%) as a light yellow oil.

### Step 4:

To a solution of tert-butyl (3R)-3-[[2-(4-pyridyl)thieno[2,3-c]pyridin-7-yl]amino]piperidine-1-carboxylate (100 mg, 219.23 μmol) and 2-fluoro-4-(triazolo[4,5-b]pyridin-3-yl)benzoyl chloride (134.78 mg, 438.46 μmol,) in THF (4 mL) was added DIEA (141.67 mg, 1.10 mmol, 190.92 μL) at 20° C. under N₂. The mixture was stirred at 60° C. for 12 h. The reaction mixture was poured into water (10 mL) and extracted with ethyl acetate (3×10 mL). The combined organic phase was washed with brine (50 mL), dried with anhydrous Na₂SO₄, and concentrated. The residue was purified by prep-HPLC (HCl condition, column: Phenomenex Luna C18 100*40 mm*5 μm; mobile phase: [H₂O (0.04% HCl)-MeCN]; gradient: 30%-65% B over 8.0 min), and the product was concentrated to give tert-butyl (3R)-3-[[2-fluoro-4-(triazolo[4,5-b]pyridin-3-yl)benzoyl]-[2-(4-pyridyl)thieno[2,3-c]pyridin-7-yl]amino]piperidine-1-carboxylate (50 mg, 22%) as a light yellow solid.

### Step 5:

A solution of tert-butyl (3R)-3-[[2-fluoro-4-(triazolo[4,5-b]pyridin-3-yl)benzoyl]-[2-(4-pyridyl)thieno[2,3-c]pyridin-7-yl]amino]piperidine-1-carboxylate (50 mg, 73.00 μmol) in 4M HCl in dioxane (2 mL) was stirred at 20° C. for 1 h and then was concentrated. The residue was purified by prep-HPLC (neutral condition, column: Waters Xbridge Prep OBD C18 150*40 mm*10 μm; mobile phase: [H₂O (10 mM NH₄HCO₃)-MeCN]; gradient: 15%-50% B over 8.0 min), and the product was lyophilized to give 2-fluoro-N-[(3R)-3-piperidyl]-N-[2-(4-pyridyl)thieno[2,3-c]pyridin-7-yl]-4-(triazolo[4,5-b]pyridin-3-yl)benzamide (22 mg, 54%) as an off-white solid. LC-MS: [M+H⁺]=551.4. ¹H NMR (400 MHZ, DMSO-d₆): δ 8.89-8.62 (m, 4H), 8.60-8.38 (m, 1H), 8.36-7.97 (m, 3H), 7.84 (br d, J=4.6 Hz, 3H), 7.81-7.41 (m, 2H), 4.72-4.49 (m, 1H), 3.29-3.19 (m, 1H), 2.93-2.70 (m, 1H), 2.37-1.92 (m, 3H), 1.88-1.38 (m, 3H), 1.35-1.07 (m, 1H).

### Synthesis of (R)-4-(3H-[1,2,3]triazolo[4,5-b]pyridin-3-yl)-2-fluoro-N-(2-(3-methoxyphenyl)thieno[3,2-c]pyridin-4-yl)-N-(piperidin-3-yl)benzamide (Example 385)

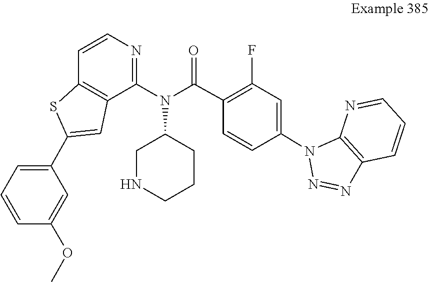 [SMILES: COc1cccc(-c2cc3c(N(C(=O)c4ccc(-n5nnc6cccnc65)cc4F)[C@@H]4CCCNC4)nccc3s2)c1]

Example 385 was prepared according to the procedure described for Example 161 by substituting 4-pyridylboronic acid with (3-methoxyphenyl) boronic acid boronic acid, provided the title compound hydrochloride salt as a white solid. LC-MS: [M+H⁺]=580.4. ¹H NMR (400 MHZ, DMSO-d₆): δ 1.04-1.19 (m, 1H), 1.66-1.87 (m, 2H), 2.05-2.17 (m, 1H), 2.69-2.77 (m, 1H), 3.14-3.24 (m, 2H), 3.76 (br d, J=10.88 Hz, 1H), 3.89 (s, 3H), 4.87-5.22 (m, 1H), 7.02-7.13 (m, 1H), 7.02-7.13 (m, 1H), 7.38-7.62 (m, 5H), 7.89 (br d, J=8.50 Hz, 1H), 7.96-8.05 (m, 2H), 8.07-8.16 (m, 1H), 8.37 (br d, J=5.50 Hz, 1H), 8.67 (d, J=8.00 Hz, 1H), 8.76-9.08 (m, 3H).

### Synthesis of N-[2-(2-cyano-4-pyridyl)thieno[3,2-c]pyridin-4-yl]-2-fluoro-N-[(3R)-3-piperidyl]-4-(triazolo[4,5-b]pyridin-3-yl)benzamide (Example 386)

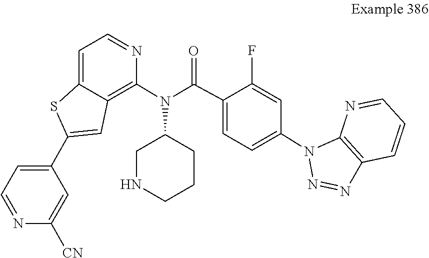 [SMILES: N#Cc1cc(-c2cc3c(N(C(=O)c4ccc(-n5nnc6cccnc65)cc4F)[C@@H]4CCCNC4)nccc3s2)ccn1]

Example 386 was prepared according to the procedure described for Example 161 by substituting 4-pyridylboronic acid with 4-(4,4,5,5-tetramethyl-1,3,2-dioxaborolan-2-yl)pyridine-2-carbonitrile, provided the title compound (10%) as an off-white solid. LC-MS: [M+H⁺]=576.4.

1H NMR (400 MHz, DMSO-d6): δ 1.04-1.21 (m, 1H), 1.73-1.94 (m, 2H), 2.07-2.29 (m, 1H), 2.69 (br s, 1H), 3.13-3.37 (m, 2H), 3.76 (br d, J=11.13 Hz, 1H), 4.97-5.23 (m, 1H), 7.59 (dd, J=8.13, 5.00 Hz, 2H), 7.89 (br d, J=8.38 Hz, 1H), 7.97 (br d, J=10.38 Hz, 1H), 8.08-8.17 (m, 2H), 8.40-8.48 (m, 1H), 8.62-8.73 (m, 2H), 8.75-9.17 (m, 5H).

### Synthesis of N-[2-(4-cyanophenyl)thieno[3,2-c]pyridin-4-yl]-2-fluoro-N-[(3R)-3-piperidyl]-4-(triazolo[4,5-b]pyridin-3-yl)benzamide (Example 387)

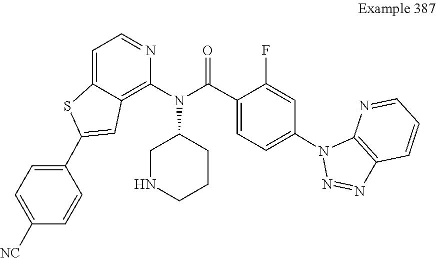 [SMILES: N#Cc1ccc(-c2cc3c(N(C(=O)c4ccc(-n5nnc6cccnc65)cc4F)[C@@H]4CCCNC4)nccc3s2)cc1]

Example 387 was prepared according to the procedure described for Example 161 by substituting 4-pyridylboronic acid with (4-cyanophenyl) boronic acid, provided the title compound hydrochloride salt (29%) as a white solid. LC-MS: [M+H⁺]=575.4. ¹H NMR (400 MHZ, DMSO-d₆): δ 9.07-8.84 (m, 2H), 8.79 (br d, J=4.0 Hz, 1H), 8.67 (br d, J=8.3 Hz, 1H), 8.43-8.36 (m, 2H), 8.17-7.96 (m, 7H), 7.88 (br d, J=8.4 Hz, 1H), 7.62-7.52 (m, 2H), 5.20-4.95 (m, 1H), 3.76 (br d, J=12.0 Hz, 1H), 3.20 (br d, J=9.5 Hz, 1H), 2.74-2.69 (m, 1H), 2.15-2.04 (m, 1H), 1.93-1.64 (m, 3H), 1.22-1.02 (m, 1H).

### Synthesis of 4-(5-amino-1-methyl-pyrazol-4-yl)-2-fluoro-N-(8-methyl-1-isoquinolyl)-N-[(3R)-3-piperidyl]benzamide (Example 388)

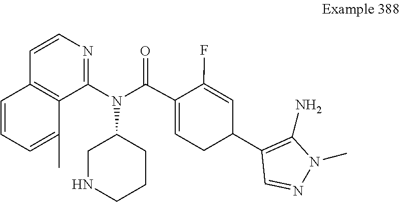 [SMILES: Cc1cccc2ccnc(N(C(=O)C3=CCC(c4cnn(C)c4N)C=C3F)[C@@H]3CCCNC3)c12]

Example 388 was prepared according to the procedure described for Example 352 by substituting 3-chloropyrazin-2-amine with 4-bromo-2-methyl-pyrazol-3-amine, provided the title compound as a white solid. LC-MS: [M+H⁺]=459.2. ¹H NMR (400 MHZ, CD₃OD) δ 8.43 (d, J=5.5 Hz, 1H), 7.97 (s, 1H), 7.78-7.65 (m, 2H), 7.58-7.42 (m, 2H), 7.07-6.82 (m, 3H), 5.12-5.00 (m, 1H), 3.99 (br dd, J=1.6, 11.9 Hz, 1H), 3.88-3.70 (m, 4H), 3.40 (br d, J=12.4 Hz, 1H), 3.06-2.92 (m, 1H), 2.91-2.78 (m, 3H), 2.00-1.83 (m, 3H), 1.47 (tt, J=8.0, 12.4 Hz, 1H).

### Synthesis of 4-(5-amino-2-methyl-1H-imidazol-4-yl)-2-fluoro-N-(8-methyl-1-isoquinolyl)-N-[(3R)-3-piperidyl]benzamide (Example 389)

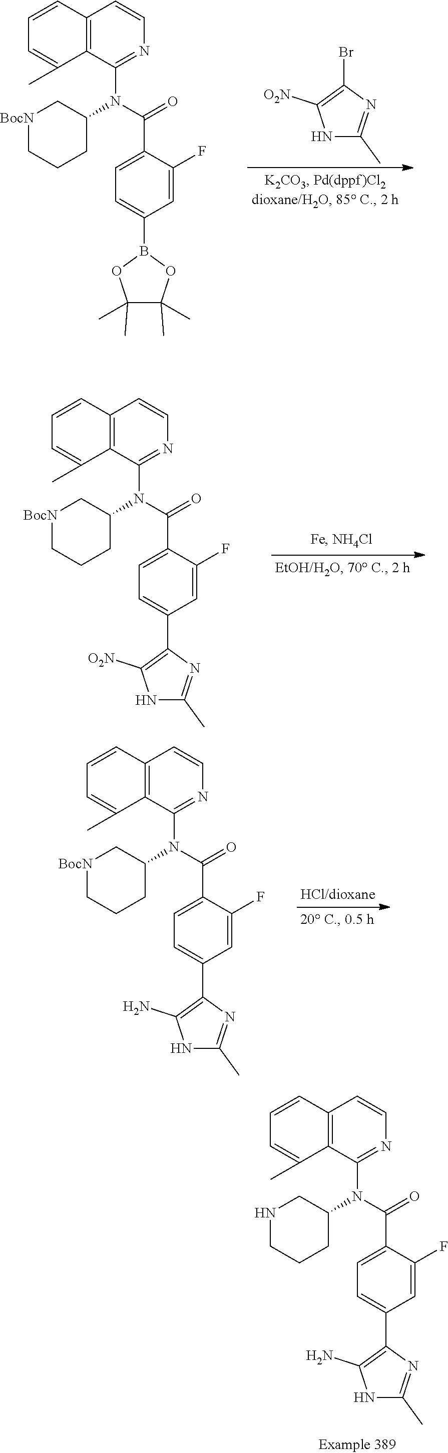

### Step 1:

To a solution of tert-butyl (3R)-3-[[2-fluoro-4-(4,4,5,5-tetramethyl-1,3,2-dioxaborolan-2-yl)benzoyl]-(8-methyl-1-isoquinolyl)amino]piperidine-1-carboxylate (200 mg, 339.27 μmol) in dioxane (2 mL) and H₂O (0.4 mL) was added 4-bromo-2-methyl-5-nitro-1H-imidazole (83.87 mg, 407.12 μmol), K₂CO₃ (140.67 mg, 1.02 mmol), and Pd(dppf)Cl₂ (24.82 mg, 33.93 μmol) at 20° C. under N₂. The reaction was heated to 85° C. and stirred for 2 h under N₂. The reaction mixture was filtered and concentrated. The residue was purified by prep-HPLC (column: Phenomenex luna C18 100*40 mm*5 μm; mobile phase: [H₂O (0.04% HCl)-MeCN]; gradient: 40%-70% B over 8.0 min) to give tert-butyl (3R)-3-[[2-fluoro-4-(2-methyl-5-nitro-1H-imidazol-4-yl)benzoyl]-(8-methyl-1-isoquinolyl)amino]piperidine-1-carboxylate (70 mg, 35%) as a yellow solid.

### Step 2:

To a solution of tert-butyl (3R)-3-[[2-fluoro-4-(2-methyl-5-nitro-1H-imidazol-4-yl)benzoyl]-(8-methyl-1-isoquinolyl)amino]piperidine-1-carboxylate (44 mg, 74.75 μmol) in EtOH (2 mL) and H₂O (0.4 mL) were added Fe (20.87 mg, 373.75 μmol) and NH₄Cl (19.99 mg, 373.75 μmol) at 20° C. under N₂. The reaction was heated to 70° C. and stirred for 3 h under N₂. The reaction mixture was filtered and concentrated. The residue was purified by prep-HPLC (column: Phenomenex luna C18 100*40 mm*5 μm; mobile phase: [H₂O (0.04% HCl)-MeCN]; gradient: 25%-55% B over 8.0 min) to give tert-butyl (3R)-3-[[4-(5-amino-2-methyl-1H-imidazol-4-yl)-2-fluoro-benzoyl]-(8-methyl-1-isoquinolyl)amino] piperidine-1-carboxylate (15 mg, 36%) as a yellow solid.

### Step 3:

The title compound hydrochloride salt was obtained as an off-white solid by deprotection Method A used in Example 97. LC-MS: [M+H⁺]=459.3. ¹H NMR (400 MHZ, CD₃OD): δ 8.46-8.40 (m, 1H), 7.77-7.70 (m, 1H), 7.69-7.62 (m, 1H), 7.55-7.45 (m, 2H), 7.05-6.96 (m, 1H), 6.94-6.83 (m, 2H), 5.11-5.00 (m, 1H), 4.03-3.95 (m, 1H), 3.85-3.75 (m, 1H), 3.71-3.63 (m, 1H), 3.58-3.47 (m, 1H), 3.05-2.93 (m, 1H), 2.91-2.79 (m, 3H), 2.46 (s, 3H), 2.01-1.84 (m, 2H), 1.60-1.26 (m, 1H).

### Synthesis of 4-(5-bromo-2-methyl-triazol-4-yl)-2-fluoro-N-(8-methyl-1-isoquinolyl)-N-[(3R)-3-piperidyl] benzamide (Example 390)

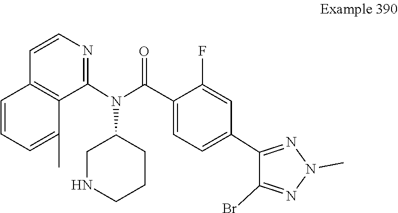 [SMILES: Cc1cccc2ccnc(N(C(=O)c3ccc(-c4nn(C)nc4Br)cc3F)[C@@H]3CCCNC3)c12]

Example 390 was prepared according to the procedure described for Example 352 by substituting 3-chloropyrazin-2-amine with 4,5-dibromo-2-methyl-triazole, provided the title compound hydrochloride salt as a yellow solid. LC-MS: [M+H⁺]=523.3. ¹H NMR (400 MHz, CD₃OD): δ 8.45-8.40 (m, 1H), 7.75-7.69 (m, 1H), 7.65 (d, J=7.9 Hz, 1H), 7.54-7.44 (m, 2H), 7.33 (dd, J=1.3, 11.4 Hz, 1H), 7.26 (dd, J=1.4, 8.0 Hz, 1H), 6.92-6.86 (m, 1H), 5.11-5.03 (m, 1H), 4.12 (s, 3H), 4.04-3.97 (m, 1H), 3.83 (br t, J=11.2 Hz, 1H), 3.72-3.64 (m, 2H), 3.58-3.48 (m, 1H), 3.11-2.96 (m, 1H), 2.89-2.81 (m, 3H), 2.02-1.88 (m, 2H), 1.61 (m, J=7.9, 11.9 Hz, 1H).

### Synthesis of 4-(3-amino-1-methyl-pyrazol-4-yl)-2-fluoro-N-(8-methyl-1-isoquinolyl)-N-[(3R)-3-piperidyl] benzamide (Example 391)

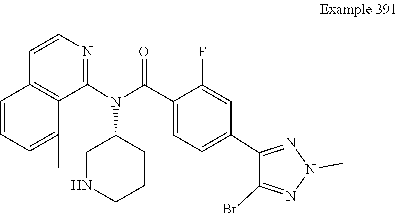 [SMILES: Cc1cccc2ccnc(N(C(=O)c3ccc(-c4nn(C)nc4Br)cc3F)[C@@H]3CCCNC3)c12]

Example 391 was prepared according to the procedure described for Example 352 by substituting 3-chloropyrazin-2-amine with 4-bromo-1-methyl-pyrazol-3-amine, provided the title compound hydrochloride salt as a yellow solid. LC-MS: [M+H⁺]=459.3. ¹H NMR (400 MHz, CD₃OD): δ 8.46-8.39 (m, 1H), 7.83 (s, 1H), 7.78-7.65 (m, 2H), 7.56-7.46 (m, 2H), 7.03-6.90 (m, 2H), 6.83 (d, J=8.3 Hz, 1H), 5.10-5.00 (m, 1H), 4.03-3.94 (m, 1H), 3.84-3.75 (m, 3H), 3.70-3.65 (m, 1H), 3.62-3.52 (m, 2H), 3.39 (br d, J=12.5 Hz, 1H), 3.20-3.08 (m, 1H), 3.06-2.92 (m, 1H), 2.91-2.81 (m, 3H), 2.75-2.63 (m, 1H), 1.96-1.84 (m, 1H), 1.55-1.17 (m, 1H).

### Synthesis of 4-(4-amino-2,5-dimethyl-pyrazol-3-yl)-2-fluoro-N-(8-methyl-1-isoquinolyl)-N-[(3R)-3-piperidyl]benzamide (Example 392)

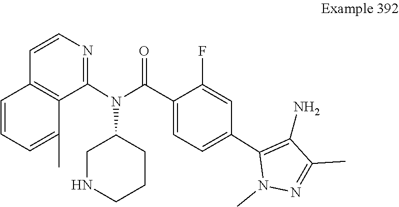 [SMILES: Cc1nn(C)c(-c2ccc(C(=O)N(c3nccc4cccc(C)c34)[C@@H]3CCCNC3)c(F)c2)c1N]

Example 392 was prepared according to the procedure described for Example 389 by substituting 4-bromo-2-methyl-5-nitro-1H-imidazole with 5-chloro-1,3-dimethyl-4-nitro-pyrazole, provided the title compound as a yellow solid. LC-MS: [M+H⁺]=473.3. ¹H NMR (400 MHZ, CD₃OD): δ ppm 8.44 (d, J=5.5 Hz, 1H), 7.76 (d, J=5.5 Hz, 1H), 7.69 (d, J=8.0 Hz, 1H), 7.58-7.46 (m, 2H), 7.07 (t, J=7.4 Hz, 1H), 6.97 (dd, J=1.4, 10.3 Hz, 1H), 6.81 (dd, J=1.5, 8.0 Hz, 1H), 5.14-5.03 (m, 1H), 4.06-3.97 (m, 1H), 3.90-3.78 (m, 1H), 3.42 (br d, J=12.5 Hz, 1H), 3.31 (s, 3H), 3.09-2.95 (m, 1H), 2.86 (s, 3H), 2.24 (s, 3H), 2.03-1.88 (m, 2H), 1.69-1.53 (m, 1H).

### Synthesis of (R)—N-(8-chloroisoquinolin-1-yl)-2-fluoro-4-(4-methyl-1H-1,2,3-triazol-1-yl)-N-(piperidin-3-yl)benzamide (Example 393)

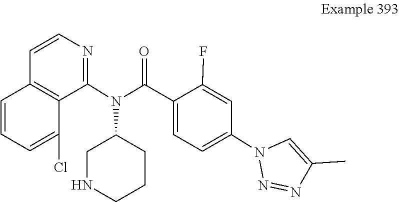 [SMILES: Cc1cn(-c2ccc(C(=O)N(c3nccc4cccc(Cl)c34)[C@@H]3CCCNC3)c(F)c2)nn1]

Example 393 was prepared according to the procedure for Example 251 by substituting tert-butyl (R)-3-((8-methylisoquinolin-1-yl)amino)piperidine-1-carboxylate with tert-butyl (R)-3-((8-chloroisoquinolin-1-yl)amino)piperidine-1-carboxylate, provided the title compound hydrochloride salt as an off-white solid. LC-MS: [M+H⁺]=465.25. ¹H NMR (DMSO-d₆ at 90° C., 400 MHZ): δ ppm 8.54 (d, J=6.0 Hz, 1H), 8.32 (s, 1H), 7.91-7.88 (m, 2H), 7.81 (d, J=7.6 Hz, 1H), 7.64 (t, J=8.0 Hz, 1H), 7.43 (d, J=9.2 Hz, 1H), 7.33 (dd, J=8.4 Hz, 1.6 Hz, 1H), 7.17 (t, J=7.6 Hz, 1H), 5.02-4.96 (m, 1H), 3.83-3.76 (m, 1H), 3.54-3.45 (m, 1H), 3.25-3.22 (m, 1H), 2.82-2.76 (m, 1H), 2.23 (s, 3H), 1.90-1.83 (m, 3H), 1.25-1.21 (m, 1H).

### Synthesis of (R)-2-fluoro-4-(5-methyl-1,3,4-oxadiazol-2-yl)-N-(8-methylisoquinolin-1-yl)-N-(piperidin-3-yl)benzamide (Example 394)

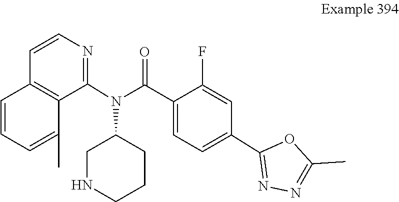 [SMILES: Cc1nnc(-c2ccc(C(=O)N(c3nccc4cccc(C)c34)[C@@H]3CCCNC3)c(F)c2)o1]

Example 394 was prepared according to the procedure for Example 127 by substituting 2-bromo-5-methyl-1,3,4-thiadiazole with 2-bromo-5-methyl-1,3,4-oxadiazole, provided the title compound formate salt as an off-white solid. LC-MS: [M−HCOOH+H⁺]=446.2. ¹H NMR (CD₃OD, 400 MHZ): δ ppm 8.42 (d, J=5.2 Hz, 1H), 7.72-7.68 (m, 1H), 7.65-7.60 (m, 1H), 7.53-7.47 (m, 2H), 7.43-7.40 (m, 1H), 7.36-7.32 (m, 1H), 7.04-7.00 (m, 1H), 5.07-5.00 (m, 1H), 4.00-3.98 (m, 1H), 3.81-3.75 (m, 1H), 3.39-3.35 (m, 1H), 3.32 (s, 3H), 3.00-2.96 (m, 1H), 2.85 (s, 3H), 1.94-1.93 (m, 3H), 1.60-1.54 (m, 1H).

### Synthesis of (R)—N-(6-(1H-pyrazol-4-yl)isoquinolin-1-yl)-4-(2-methyl-2H-tetrazol-5-yl)-N-(piperidin-3-yl)benzamide (Example 395)

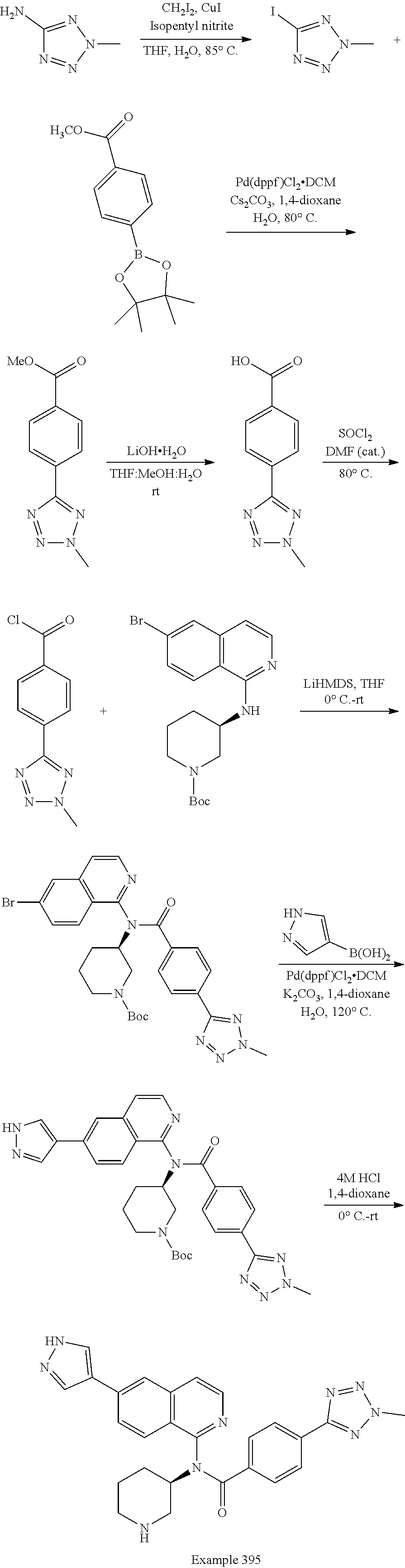

### Step 1:

To a stirred solution of 2-methyl-2H-tetrazol-5-amine (1 g, 10.09 mmol) and diiodomethane (3.2 mL g, 40.36 mmol) in THF (10 mL) and water (0.5 mL) were added CuI (1.9 g, 10.09 mmol) and isopentyl nitrite (4.74 g, 35.31 mmol) at room temperature. The reaction mixture was stirred at 85° C. for 2 h. The reaction mixture was cooled to room temperature and diluted with NH₄OH and extracted with EtOAc (2×100 mL). The combined organic layer was washed with brine, dried over Na₂SO₄, and concentrated. The residue was purified by Combi-flash using 10-15% EtOAc in hexane to afford 5-iodo-2-methyl-2H-tetrazole (1.3 g, 61.61%) as an off-white solid. LC-MS: [M+H⁺]=211.0.

### Step 2:

To a stirred solution of 5-iodo-2-methyl-2H-tetrazole (500 mg, 2.381 mmol) and methyl 4-(4,4,5,5-tetramethyl-1,3,2-dioxaborolan-2-yl)benzoate (0.936 g, 3.57 mmol) in 1,4-dioxane (5 mL) and water (0.5 mL) were added Cs₂CO₃ (2.32 g, 7.14 mmol) at room temperature. The reaction mixture was purged with N₂ for 10 minutes. Then PdCl₂ (dppf)-CH₂Cl₂ (97 mg, 0.119 mmol) was added to the reaction mixture, and the reaction was stirred at 80° C. for 16 h. The reaction mixture was cooled to room temperature, diluted with EtOAc (100 mL), and filtered through a celite bed. The filtrate was washed with brine, dried over Na₂SO₄, and concentrated. The residue was purified by Combi-flash using 50-60% EtOAc in hexane to afford methyl 4-(2-methyl-2H-tetrazol-5-yl) benzoate (400 mg, 77%) as an off-white solid. LC-MS: [M+H⁺]=219.2.

### Step 3:

To a solution of methyl 4-(2-methyl-2H-tetrazol-5-yl) benzoate (400 mg, 1.833 mmol) in methanol (5 mL), THF (5 mL), and water (2 mL) was added LiOH·H₂O (137 mg, 5.499 mmol) at room temperature, and the reaction was stirred for 16 h. The reaction mixture was concentrated and acidified with 1N aq. HCl to pH˜2. The precipitate was collected by filtration and dried to afford 4-(2-methyl-2H-tetrazol-5-yl) benzoic acid (370 mg, 96%) as an off-white solid. LC-MS: [M+H⁺]=205.15.

### Step 4:

To a solution of 4-(2-methyl-2H-tetrazol-5-yl) benzoic acid (350 mg, 1.714 mmol) and SOCl₂ (10 mL, 137 mmol) was added catalytic amount of DMF (0.05 mL) at room temperature, and the reaction mixture was stirred at 80° C. for 16 h. The reaction mixture was concentrated to afford 4-(2-methyl-2H-tetrazol-5-yl)benzoyl chloride (280 mg), which was used in the next step without further purification.

### Step 5:

To a solution of tert-butyl (R)-3-((6-bromoisoquinolin-1-yl) amino) piperidine-1-carboxylate (250 mg, 0.615 mmol) in THF (10 mL) at 0° C. was added LiHMDS in THF (0.879 mL, 1.231 mmol), and the reaction was stirred for 20 min. Then 4-(2-methyl-2H-tetrazol-5-yl)benzoyl chloride (274 mg, 1.231 mmol) in THF (5 mL) was added to the reaction mixture at 0° C., and the reaction was stirred for 10 minutes. The reaction mixture was quenched with saturated NH₄Cl (10 mL) and extracted with EtOAc (30 mL). The organic layer was dried over Na₂SO₄ and concentrated. The residue was purified by prep-HPLC (Conditions: LUNA (250×21.2×5 μm), Mobile phase A: 0.1% formic acid in water (100%), Mobile phase B: 100% MeCN, Gradient: Time (min)/% B: 0/65, 10/90, Flow: 15 mL/min, Dilution: MeCN+Water). Pure fractions were combined and concentrated to afford tert-butyl (R)-3-(N-(6-bromoisoquinolin-1-yl)-4-(2-methyl-2H-tetrazol-5-yl)benzamido)piperidine-1-carboxylate (180 mg, 74.6%) as an off-white solid.

### Step 6:

To a solution of tert-butyl (R)-3-(N-(6-bromoisoquinolin-1-yl)-4-(2-methyl-2H-tetrazol-5-yl)benzamido)piperidine-1-carboxylate (100 mg, 0.169 mmol) in 1,4-dioxane (1 mL) and water (1 mL) were added (1H-pyrazol-4-yl)boronic acid (37.8 mg, 0.338 mmol) and potassium carbonate (70 mg, 0.506 mmol) at room temperature, and the reaction mixture was purged with N₂ for 10 minutes. Then PdCl₂(dppf)-CH₂Cl₂ (13.78 mg, 0.017 mmol) was added to the reaction mixture, and the reaction was stirred at 120° C. for 16 h. The reaction mixture was cooled, diluted with EtOAc (20 mL), and filtered through a celite bed. The filtrate was concentrated, and the residue was purified by Prep-HPLC (Conditions: LUNA (250×21.2×5 μm), Mobile phase A: 0.1% formic acid in water (100%), Mobile phase B: 100% MeCN, Gradient: Time (min)/% B: 0/60, 10/75, Flow: 15 mL/min, Dilution: MeCN+Water). Pure fractions were combined and concentrated to afford tert-butyl (R)-3-(N-(6-(1H-pyrazol-4-yl)isoquinolin-1-yl)-4-(2-methyl-2H-tetrazol-5-yl)benzamido)piperidine-1-carboxylate (31 mg) as an off-white solid. LC-MS: [M+H⁺]=580.1.

### Step 7:

The title compound hydrochloride salt was obtained as an off-white solid by deprotection Method A used in Example 97. LC-MS: [M+H⁺]=480.2. ¹H NMR (CD₃OD, 400 MHZ): δ 8.46-8.45 (m, 1H), 8.24 (s, 2H), 8.06-7.99 (m, 2H), 7.92-7.90 (m, 1H), 7.78-7.71 (m, 3H), 7.42-7.36 (m, 2H), 5.32-5.29 (m, 1H), 4.30 (s, 3H), 3.93-3.90 (m, 1H), 3.74-3.68 (m, 1H), 2.90-2.87 (m, 1H), 2.00-1.90 (m, 3H), 1.38-1.25 (m, 1H), 0.96-0.87 (m, 1H).

### Synthesis of (R)-4-(3H-[1,2,3]triazolo[4,5-b]pyridin-3-yl)-N-(6-ethynylisoquinolin-1-yl)-2-fluoro-N-(piperidin-3-yl)benzamide (Example 396)

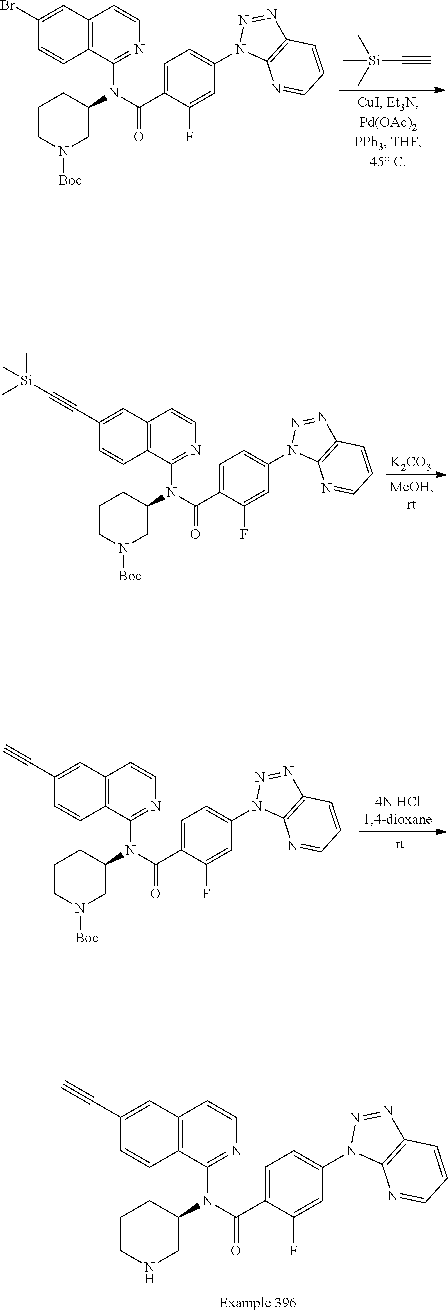

### Step 1:

To a solution of tert-butyl (R)-3-(4-(3H-[1,2,3]triazolo[4,5-b]pyridin-3-yl)-N-(6-bromoisoquinolin-1-yl)-2-fluorobenzamido)piperidine-1-carboxyla (300 mg, 0.464 mmol) in THF (3 mL) was added triethylamine (1.29 mL, 9.28 mmol), copper(I) iodide (17.67 mg, 0.093 mmol), and triphenylphosphine (24.34 mg, 0.093 mmol) at room temperature. The reaction mixture was degassed with nitrogen for 5 min. Then Pd(OAc)₂ (10.42 mg, 0.046 mmol) and ethynyltrimethylsilane (0.661 mL, 4.64 mmol) were added to the reaction mixture. The reaction was stirred at 45° C. for 16 h. The reaction mixture was diluted with water (30 mL) and extracted with EtOAc (2×50 mL). The organic layer was washed with brine, dried over Na₂SO₄, and concentrated to afford tert-butyl (R)-3-(4-(3H-[1,2,3]triazolo[4,5-b]pyridin-3-yl)-2-fluoro-N-(6-((trimethylsilyl)ethynyl)isoquinolin-1-yl)benzamido)piperidine-1-carboxylate (300 mg) as a brown solid, which was used in the next step directly. LC-MS: [M+H⁺]=664.1.

### Step 2:

To a solution of tert-butyl (R)-3-(4-(3H-[1,2,3]triazolo[4,5-b]pyridin-3-yl)-2-fluoro-N-(6-((trimethylsilyl)ethynyl)isoquinolin-1-yl)benzamido)piperidine-1-carboxylate (300 mg, 0.452 mmol) in MeOH (2 mL) at room temperature was added K₂CO₃ (187 mg, 1.356 mmol). The reaction was stirred for 2 h, after which it was diluted with H₂Or (50 mL) and extracted with EtOAc (2×50 mL). The combined organic layer was washed with brine, dried over Na₂SO₄, and concentrated. The residue was purified by Combi-flash using 10% EtOAc in hexane and was further purified by Prep-HPLCHPLC (Condition: X-SELECT CSH, Mobile phase A: 0.1% formic acid in water (100%), Mobile phase B: 100% MeCN, Gradient: Time (min)/% B: 0/65, 10/85, Flow: 15 mL/min, Dilution: THF+MeCN+H₂O). The pure fractions were combined and dried by lyophilization to afford tert-butyl (R)-3-(4-(3H-[1,2,3]-triazolo[4,5-b]pyridin-3-yl)-N-(6-ethynylisoquinolin-1-yl)-2-fluorobenzamido) piperidine-1-carboxylate formate salt (40 mg, 15%) as an off-white solid. LC-MS: [M+H⁺]=592.3.

### Step 3:

The title compound hydrochloride salt was obtained as an off-white solid by deprotection Method A used in Example 97. LC-MS: [M+H⁺]=492.2. ¹H NMR (CD3OD, 400 MHZ): δ 8.74 (d, J=3.6 Hz, 1H), 8.49-8.43 (m, 2H), 8.17 (d, J=8.4 Hz, 1H), 8.06-8.01 (m, 1H), 7.97-7.91 (m, 2H), 7.78-7.72 (m, 2H), 7.53-7.51 (m, 1H), 7.40-7.29 (m, 1H), 5.29-5.23 (m, 1H), 3.99-3.96 (m, 1H), 3.74-3.68 (m, 1H), 3.69-3.65 (m, 1H), 3.27-3.25 (m, 1H), 2.87-2.86 (m, 1H), 1.92-1.90 (m, 3H), 1.30-1.28 (m, 1H).

### Synthesis of (R)-2-fluoro-N-(piperidin-3-yl)-N-(6-(pyridin-3-yl)isoquinolin-1-yl)-4-(pyrimidin-2-ylamino)benzamide (Example 397)

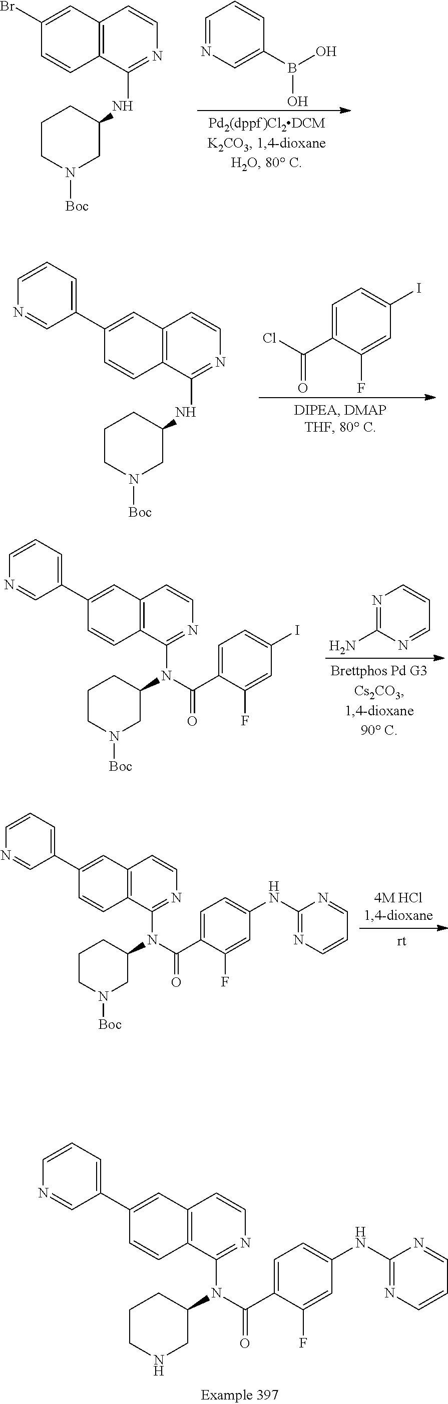

### Step 1:

To a stirred solution of tert-butyl (R)-3-((6-bromoisoquinolin-1-yl)amino)piperidine-1-carboxylate (500 mg, 1.231 mmol) in 1,4-dioxane (3 mL) and water (0.1 mL) were added pyridin-3-ylboronic acid (303 mg, 2.461 mmol) and potassium carbonate (510 mg, 3.69 mmol). The reaction mixture was purged with nitrogen for 10 minutes. Then, PdCl₂ (dppf)-CH₂Cl₂ (100 mg, 0.123 mmol) was added to the reaction mixture, and the reaction was stirred at 90° C. for 16 h. The reaction mixture was diluted with EtOAc (50 mL), filtered through celite, and washed with water (30 mL). The organic layer was washed with brine, dried over Na₂SO₄, and concentrated. The residue was purified by cCombi-flash, eluting with 45% EtOAc in Hexane to afford tert-butyl (R)-3-((6-(pyridin-3-yl)isoquinolin-1-yl)amino)piperidine-1-carboxylate (400 mg, 76%) as a brown solid. LC-MS: [M+H⁺]=405.2.

### Step 2:

To a solution of tert-butyl (R)-3-((6-(pyridin-3-yl)isoquinolin-1-yl)amino)piperidine-1-carboxylate (400 mg, 0.989 mmol) and 2-fluoro-4-iodobenzoyl chloride (563 mg, 1.978 mmol) in THF (5 mL) at room temperature were added DIPEA (1.03 mL, 5.93 mmol) and DMAP (12.08 mg, 0.099 mmol), and the reaction was stirred at 80° C. for 16 h. The reaction mixture was diluted with water and extracted with EtOAc (2×20 mL). The combined organic layer was washed with brine, dried over Na₂SO₄, and concentrated. The residue was purified by Combi-flash, eluting with 60% EtOAc in hexane first and further purified by prep HPLC (Condition: Luna OMEGA C-18 (250×21.2×5 μm)), Mobile phase A: 0.1% formic acid in water, Mobile phase B: MeCN (100%), T/% B: 0/60, 10/85, Flow: 15 mL/min, Dilution: MeCN+H₂O). The pure fractions were dried by lyophilization to afford tert-butyl (R)-3-(2-fluoro-4-iodo-N-(6-(pyridin-3-yl)isoquinolin-1-yl)benzamido)piperidine-1-carboxylate (100 mg, 62%) as an off-white solid. LC-MS: [M+H⁺]=653.1.

### Step 3:

To a stirred solution of tert-butyl (R)-3-(2-fluoro-4-iodo-N-(6-(pyridin-3-yl)isoquinolin-1-yl)benzamido)piperidine-1-carboxylate (100 mg, 0.153 mmol) in 1,4-dioxane (1 mL) at room temperature were added Cs₂CO₃ (74.9 mg, 0.230 mmol) and pyrimidin-2-amine (21.86 mg, 0.230 mmol). The reaction mixture was degassed with nitrogen for 10 minutes. Then, Brettphos Pd G3 (27.8 mg, 0.031 mmol) was added to reaction mixture, and the reaction was stirred at 90° C. for 16 h. The reaction mixture was diluted with water (20 mL) and extracted with EtOAc (2×20 mL). The combined organic layer was washed with brine (10 mL), dried over Na₂SO₄, and concentrated. The residue was purified by Prep-HPLC (Condition: Luna OMEGA C-18 (250×21.2×5 μm), Mobile phase A: 0.1% formic acid in H₂O, Mobile phase B: MeCN (100%), T/% B: 0/60, 10/85, Flow: 15 mL/min, Dilution: 15 mL/min). The pure fractions were dried by lyophilization to afford tert-butyl (R)-3-(2-fluoro-N-(6-(pyridin-3-yl)isoquinolin-1-yl)-4-(pyrimidin-2-ylamino)benzamido)piperidine-1-carboxylate (30 mg) as an off-white solid.

### Step 4:

The title compound hydrochloride salt was obtained as an off-white solid by deprotection Method A used in Example 97. LC-MS: [M+H⁺]=520.2. ¹H NMR (CD₃OD, 400 MHz): δ 9.35 (s, 1H), 9.07 (d, J=7.2 Hz, 1H), 8.92 (d, J=4.8 Hz, 1H), 8.58-8.51 (m, 3H), 8.41 (s, 1H), 8.32 (d, J=7.6 Hz, 1H), 8.25-8.14 (m, 2H), 7.95-7.93 (m, 1H), 7.47-7.44 (m, 1H), 7.01-6.97 (m, 3H), 5.32-5.30 (m, 1H), 3.94-3.91 (m, 1H), 3.74-3.68 (m, 1H), 3.36-3.31 (m, 1H), 2.89-2.83 (m, 1H), 2.03-1.90 (m, 3H), 1.31-1.28 (m, 1H).

### Synthesis of (R)-4-(3H-[1,2,3]triazolo[4,5-b]pyridin-3-yl)-2-fluoro-N-(6-(3-hydroxyprop-1-yn-1-yl)isoquinolin-1-yl)-N-(piperidin-3-yl)benzamide (Example 398)

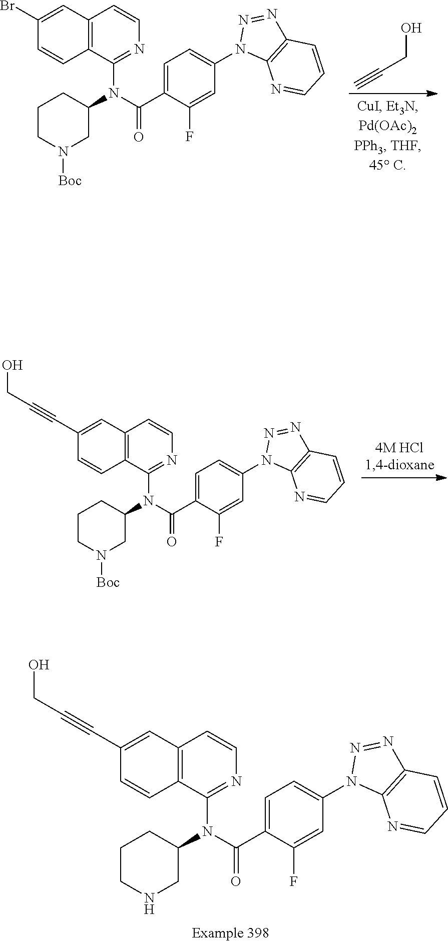

Example 398 was prepared according to the procedure described for Example 102 by substituting (R)-3-(4-(3H-[1,2,3]triazolo[4,5-b]pyridin-3-yl)-N-(7-bromoisoquinolin-1-yl)-2-fluorobenzamido)piperidine-1-carboxylate with (R)-3-(4-(3H-[1,2,3]triazolo[4,5-b]pyridin-3-yl)-N-(6-bromoisoquinolin-1-yl)-2-fluorobenzamido)piperidine-1-carboxylate, provided the title compound hydrochloride salt as an off-white solid. LC-MS: [M+H⁺]=522.2. ¹H NMR (CD₃OD, 400 MHZ): δ 8.75 (d, J=3.2 Hz, 1H), 8.51-8.42 (m, 2H), 8.15 (d, J=8.8 Hz, 1H), 8.04 (d, J=10.8 Hz, 1H), 7.95-7.90 (m, 2H), 7.73-7.68 (m, 2H), 7.54-7.51 (m, 1H), 7.32-7.28 (m, 1H), 5.29-5.26 (m, 1H), 4.40 (s, 2H), 3.94-3.93 (m, 1H), 3.72-3.66 (m, 1H), 3.36-3.31 (m, 1H), 2.89-2.83 (m, 1H), 2.03-1.90 (m, 3H), 1.34-1.28 (m, 1H).

### Synthesis of 3-acrylamido-N-(8-methylisoquinolin-1-yl)-N—((R)-piperidin-3-yl)piperidine-1-carboxamide (Example 399)

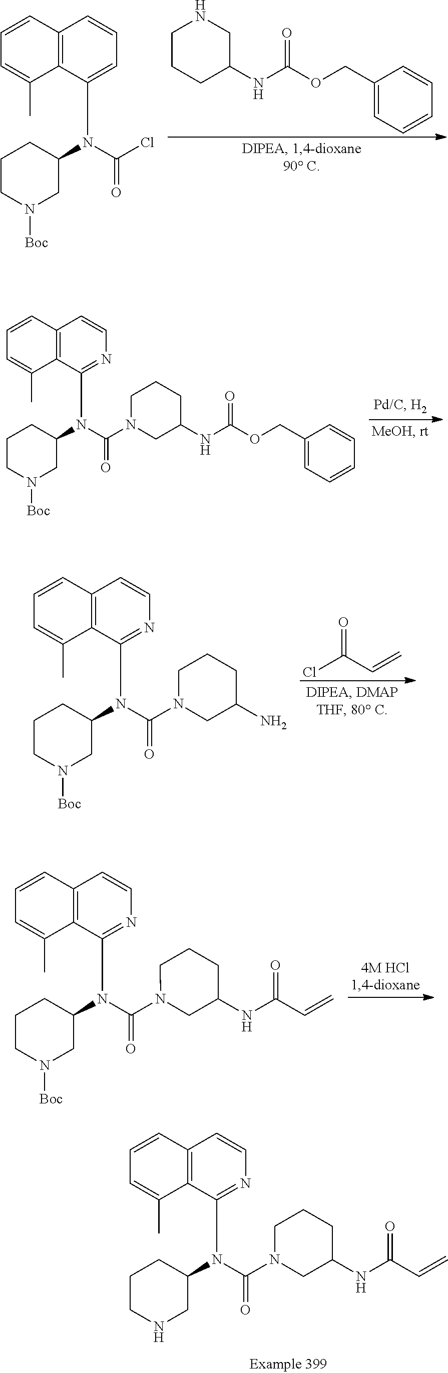

### Step 1:

To a stirred solution of benzyl piperidin-3-ylcarbamate (69.6 mg, 0.297 mmol) in 1,4-dioxane (2 mL) at 0° C. were added DIPEA (0.130 mL, 0.743 mmol) and tert-butyl (R)-3-((chlorocarbonyl)(8-methylisoquinolin-1-yl)amino)piperidine-1-carboxylate (100 mg, 0.248 mmol). The reaction was heated to 90° C. and stirred for 16 h. The reaction mixture was then diluted with water (30 mL) and extracted with EtOAc (2×30 mL). The combined organic layer was washed with brine, dried over Na₂SO₄, and concentrated. The residue was purified by Combi-Flash (eluting with 30% EtOAc in hexane) to afford tert-butyl (3R)-3-(3-(((benzyloxy)carbonyl)amino)-N-(8-methylisoquinolin-1-yl)piperidine-1-carboxamido)piperidine-1-carboxylate (110 mg, 73.4%) as an off-white solid. LC-MS: [M+H⁺]=602.35.

### Step 2:

To a stirred solution of tert-butyl (3R)-3-(3-(((benzyloxy) carbonyl)amino)-N-(8-methylisoquinolin-1-yl)piperidine-1-carboxamido)piperidine-1-carboxylate (110 mg, 0.183 mmol) in MeOH (1 mL)) was added 10 wt % Pd/C (195 mg, 0.183 mmol) at room temperature. The reaction mixture was stirred under an H₂ balloon for 2 h before it was filtrated through a pad of celite. The filtrate was concentrated to afford tert-butyl (3R)-3-(3-amino-N-(8-methylisoquinolin-1-yl)piperidine-1-carboxamido)piperidine-1-carboxylate (90 mg) as solid, which was used in the next step directly. LC-MS: [M+H⁺]=468.3.

### Step 3:

To a stirred solution of tert-butyl (3R)-3-(3-amino-N-(8-methylisoquinolin-1-yl)piperidine-1-carboxamido)piperidine-1-carboxylate (90 mg, 0.192 mmol) in THF (5 mL) at 25° C. was added DIPEA (0.134 mL, 0.770 mmol), 4-DMAP (2.351 mg, 0.019 mmol), and acryloyl chloride (0.03 mL, 0.192 mmol). The reaction was stirred at 80° C. for 16 h. The reaction was diluted with water (10 mL) and extracted with EtOAc (2×10 mL). The combined organic layer was washed with brine, dried over Na₂SO₄, and concentrated. The residue was purified by Prep-HPLC (Condition: LUNA (250×21.2×5 μm), Mobile phase A: 0.1% formic acid in water (100%), Mobile phase B: 100% MeCN, Gradient: T/% B: 0/55, 10/75, Flow: 15 mL/min, Dilution: MeCN+H₂O). The pure fractions were dried to afford tert-butyl (3R)-3-(3-acrylamido-N-(8-methylisoquinolin-1-yl)piperidine-1-carboxamido)piperidine-1-carboxylate (35 mg, 49%) as an off-white solid. LC-MS: [M+H⁺]=522.3.

### Step 4:

The title compound hydrochloride salt was obtained as an off-white solid by deprotection Method A used in Example 97. LC-MS: [M+H⁺]=422.3. ¹H NMR (CD₃OD, 400 MHZ): δ 8.37-8.27 (m, 1H), 7.87-7.78 (m, 2H), 7.71-7.66 (m, 1H), 7.60-7.52 (m, 1H), 6.19-6.09 (m, 2H), 4.80-4.40 (m, 1H), 3.76-3.59 (m, 2H), 3.40-3.20 (m, 2H), 2.99-2.84 (m, 1H), 2.69-2.65 (m, 5H), 2.32-2.23 (m, 2H), 1.90-1.68 (m, 4H), 1.28-1.22 (m, 2H), 1.20-0.82 (m, 2H).

Syntheses of methyl (R)-2-((3-fluoro-4-((8-methylisoquinolin-1-yl)(piperidin-3-yl)carbamoyl)phenyl)amino)pyrimidine-4-carboxylate (Example 400) and (R)-2-((3-fluoro-4-((8-methylisoquinolin-1-yl)(piperidin-3-yl)carbamoyl)phenyl)amino)pyrimidine-4-carboxylic acid (Example 401)

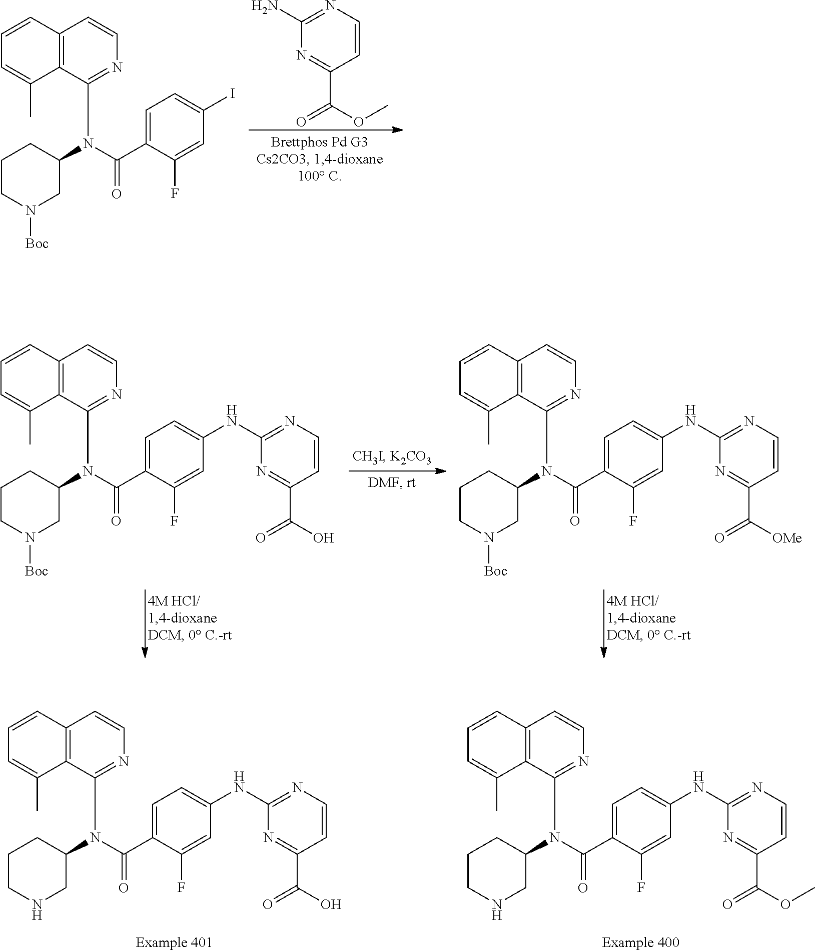 [SMILES: Cc1cccc2ccnc(N(C(=O)c3ccc(I)cc3F)[C@@H]3CCCN(C(=O)OC(C)(C)C)C3)c12 ; COC(=O)c1ccnc(N)n1 ; Cc1cccc2ccnc(N(C(=O)c3ccc(Nc4nccc(C(=O)O)n4)cc3F)[C@@H]3CCCN(C(=O)OC(C)(C)C)C3)c12 ; COC(=O)c1ccnc(Nc2ccc(C(=O)N(c3nccc4cccc(C)c34)[C@@H]3CCCN(C(=O)OC(C)(C)C)C3)c(F)c2)n1 ; Cc1cccc2ccnc(N(C(=O)c3ccc(Nc4nccc(C(=O)O)n4)cc3F)[C@@H]3CCCNC3)c12 ; COC(=O)c1ccnc(Nc2ccc(C(=O)N(c3nccc4cccc(C)c34)[C@@H]3CCCNC3)c(F)c2)n1]

### Step 1:

To a stirred solution of tert-butyl (R)-3-(2-fluoro-4-iodo-N-(8-methylisoquinolin-1-yl) benzamido) piperidine-1-carboxylate (500 mg, 0.848 mmol) in 1,4-dioxane (10 mL) were added methyl 2-aminopyrimidine-4-carboxylate (260 mg, 1.696 mmol) and Cs₂CO₃ (829 mg, 2.54 mmol) at room temperature, and the reaction mixture was purged with nitrogen for 10 min. Then, Brettphos Pd G3 (154 mg, 0.170 mmol) was added to the reaction mixture, and the reaction was stirred at 100° C. for 16 h. The reaction was cooled to room temperature, diluted with ice cold water (20 mL), and extracted with EtOAc (2×50 mL). The combined organic layer was dried over Na₂SO₄ and concentrated to afford (R)-2-((4-((1-(tert-butoxycarbonyl)piperidin-3-yl)(8-methylisoquinolin-1-yl)carbamoyl)-3-fluorophenyl) amino)pyrimidine-4-carboxylic acid (400 mg, 43.2%) as a brown solid. LC-MS: [M+H⁺]=601.3.

### Step 2:

To a solution of (R)-2-((4-((1-(tert-butoxycarbonyl)piperidin-3-yl) (8-methylisoquinolin-1-yl)carbamoyl)-3-fluorophenyl) amino) pyrimidine-4-carboxylic acid (320 mg, 0.533 mmol) in DMF (5 mL) at room temperature were added K₂CO₃ (74 mg, 0.533 mmol) and Mel (0.1 mL, 1.598 mmol), and the reaction was stirred for 16 h. The reaction mixture was quenched with saturated NH₄Cl (10 mL) and extracted with EtOAc (2×50 mL). The combined organic layer was washed with brine, dried over Na₂SO₄, and concentrated. The residue was purified by Combi-flash using 40% EtOAc in hexane first and further purified by Prep-HPLC (Conditions: Kinetex Evo C-18 (250×21.2×5 μm), Mobile phase A: 0.1% formic acid in water, Mobile phase B: MeCN (100%), Gradient: T/% B: 0/60, 10/85, Flow: 15 mL/min, Dilution: MeCN+H₂O+THF). Pure fractions were concentrated to afford methyl (R)-2-((4-((1-(tert-butoxycarbonyl)piperidin-3-yl)(8-methylisoquinolin-1-yl)carbamoyl)-3-fluorophenyl)amino)-pyrimidine-4-carboxylate (25 mg, 49.5%) as an off-white solid. LC-MS: [M+H⁺]=615.3.

### Step 3:

To a solution of methyl (R)-2-((4-((1-(tert-butoxycarbonyl)piperidin-3-yl)(8-methylisoquinolin-1-yl)carbamoyl)-3-fluorophenyl)amino)pyrimidine-4-carboxylate (20 mg, 0.033 mmol) in DCM (2 mL) at 0° C. was added 4M HCl in 1,4-dioxane (1 mL, 4 mmol), and the reaction was warmed to room temperature and stirred for 2 h. The reaction mixture was concentrated and dried by lyophilization to afford methyl (R)-2-((3-fluoro-4-((8-methylisoquinolin-1-yl)(piperidin-3-yl)carbamoyl)phenyl)amino)pyrimidine-4-carboxylate hydrochloride salt (Example 400, 18 mg, 96%) as an off-white solid. LC-MS: [M+H⁺]=515.2.

1H NMR (CD₃OD, 400 MHZ): δ 8.63 (d, J=4.8 Hz, 1H), 8.44 (d, J=5.6 Hz, 1H), 7.74-7.70 (m, 1H), 7.67-7.61 (m, 2H), 7.51-7.47 (m, 1H), 7.45-7.43 (m, 1H), 7.38 (d, J=4.8 Hz, 1H), 6.89-6.86 (m, 1H), 6.66-6.62 (m, 1H), 5.09-5.03 (m, 1H), 3.94 (s, 3H), 3.84-3.79 (m, 1H), 3.41-3.38 (m, 1H), 3.01-2.92 (m, 1H), 2.80 (s, 3H), 2.69-2.67 (m, 1H), 1.98-1.87 (m, 3H), 1.59-1.55 (m, 1H).

### Step 4:

[(R)-2-((3-fluoro-4-((8-methylisoquinolin-1-yl)(piperidin-3-yl)carbamoyl)phenyl)amino)-pyrimidine-4-carboxylic acid hydrochloride salt (Example 401) was prepared by the same method as Example 400 above to give an off-white solid. LC-MS: [M+H⁺]=501.2. ¹H NMR (CD₃OD, 400 MHZ): δ 8.63 (d, J=4.8 Hz, 1H), 8.44-8.40 (m, 1H), 7.74-7.70 (m, 1H), 7.67-7.56 (m, 2H), 7.51-7.47 (m, 1H), 7.44-7.39 (m, 2H), 6.89 (d, J=8.4 Hz, 1H), 6.66-6.60 (m, 1H), 5.09-5.03 (m, 1H), 3.96-3.94 (m, 1H), 3.84-3.79 (m, 1H), 3.37-3.34 (m, 1H), 3.30-3.28 (m, 1H), 2.80 (s, 3H), 2.69-2.61 (m, 1H), 1.98-1.87 (m, 2H), 1.59-1.55 (m, 1H).

### Synthesis of (R)-2-((3-fluoro-4-((8-methylisoquinolin-1-yl)(piperidin-3-yl)carbamoyl)phenyl)amino)pyrimidine-4-carboxamide (Example 402)

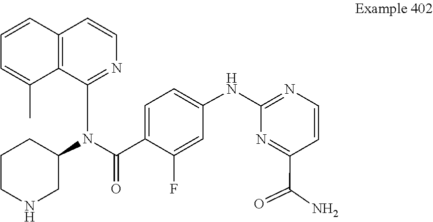 [SMILES: Cc1cccc2ccnc(N(C(=O)c3ccc(Nc4nccc(C(N)=O)n4)cc3F)[C@@H]3CCCNC3)c12]

Example 402 was prepared according to the procedure described for Example 408 by substituting 4-morpholinopyrimidin-2-amine with 2-amino-4-pyrimidinecarboxamide, provided title compound hydrochloride salt as an off-white solid. LC-MS: [M+H⁺]=500.3. ¹H NMR (CD₃OD, 400 MHZ): δ 8.63 (d, J=4.8 Hz, 1H), 8.44-8.40 (m, 1H), 7.74-7.70 (m, 1H), 7.67-7.62 (m, 1H), 7.51-7.38 (m, 4H), 6.88-6.86 (m, 1H), 6.69-6.65 (m, 1H), 5.10-5.04 (m, 1H), 3.97-3.94 (m, 1H), 3.85-3.79 (m, 1H), 3.44-3.38 (m, 1H), 3.03-2.99 (m, 1H), 2.80 (s, 3H), 2.73-2.68 (m, 1H), 1.95-1.91 (m, 2H), 1.60-1.56 (m, 1H).

### Synthesis of 4-(1-methyltriazol-4-yl)-N-[(3R)-3-piperidyl]-N-[8-[2-(3-pyridyl)ethyl]-1-isoquinolyl]benzamide (Example 403)

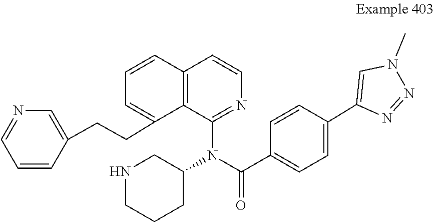 [SMILES: Cn1cc(-c2ccc(C(=O)N(c3nccc4cccc(CCc5cccnc5)c34)[C@@H]3CCCNC3)cc2)nn1]

Example 403 was prepared by hydrogenation from Example 349 under the reaction conditions (10 wt % Pd/C, H₂, MeOH, 25° C., 12 h) to provide the title compound as a white solid. LC-MS: [M+H⁺]=518.3. ¹H NMR (400 MHZ, CD₃OD): δ 8.93-8.58 (m, 2H), 8.58-8.38 (m, 2H), 8.15 (br s, 1H), 8.07-7.91 (m, 1H), 7.89-7.67 (m, 2H), 7.56 (br d, J=19.5 Hz, 2H), 7.39 (br s, 2H), 7.01 (br d, J=4.9 Hz, 2H), 5.18-4.99 (m, 1H), 4.20-4.00 (m, 3H), 3.98-3.77 (m, 2H), 3.75-3.48 (m, 4H), 3.46-3.39 (m, 1H), 3.01 (br d, J=5.3 Hz, 1H), 2.14-1.64 (m, 4H), 1.52-1.23 (m, 1H).

### Synthesis of (R)-2-fluoro-N-(8-methylisoquinolin-1-yl)-4-((4-morpholinopyrimidin-2-yl)amino)-N-(piperidin-3-yl)benzamide (Example 408)

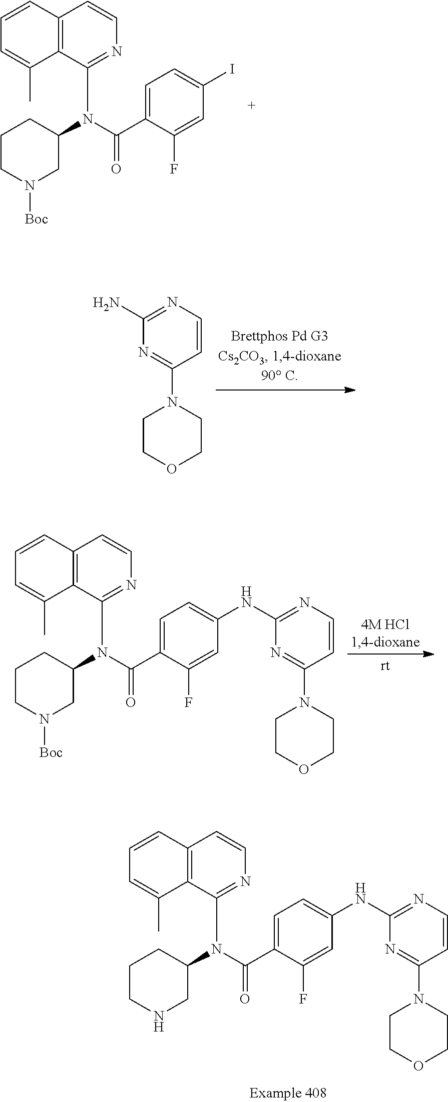 [SMILES: Cc1cccc2ccnc(N(C(=O)c3ccc(I)cc3F)[C@@H]3CCCN(C(=O)OC(C)(C)C)C3)c12 ; Nc1nccc(N2CCOCC2)n1 ; Cc1cccc2ccnc(N(C(=O)c3ccc(Nc4nccc(N5CCOCC5)n4)cc3F)[C@@H]3CCCN(C(=O)OC(C)(C)C)C3)c12 ; Cc1cccc2ccnc(N(C(=O)c3ccc(Nc4nccc(N5CCOCC5)n4)cc3F)[C@@H]3CCCNC3)c12]

Example 408 was prepared according to the procedure described for Example 131 by substituting by 3-amino-1-methyl-pyridine-2-one with 4-morpholinopyrimidin-2-amine, provided the title compound hydrochloride salt as an off-white solid. LC-MS: [M+H⁺]=542.1. ¹H NMR (CD₃OD, 400 MHZ): δ 8.43-8.40 (m, 1H), 7.79-7.64 (m, 3H), 7.53-7.45 (m, 2H), 7.09-7.02 (m, 1H), 6.86-6.78 (m, 2H), 6.58 (d, J=7.6 Hz, 1H), 5.08-4.95 (m, 1H), 3.98-3.96 (m, 1H), 3.83-3.72 (m, 8H), 3.53-3.38 (m, 1H), 3.02-2.94 (m, 1H), 2.81 (s, 3H), 2.70-2.68 (m, 1H), 1.96-1.88 (m, 3H), 1.56-1.52 (m, 1H).

### Synthesis of (R)—N-(6-(1H-tetrazol-5-yl)isoquinolin-1-yl)-2-fluoro-4-(1-methyl-1H-1,2,3-triazol-4-yl)-N-(piperidin-3-yl)benzamide (Example 405)

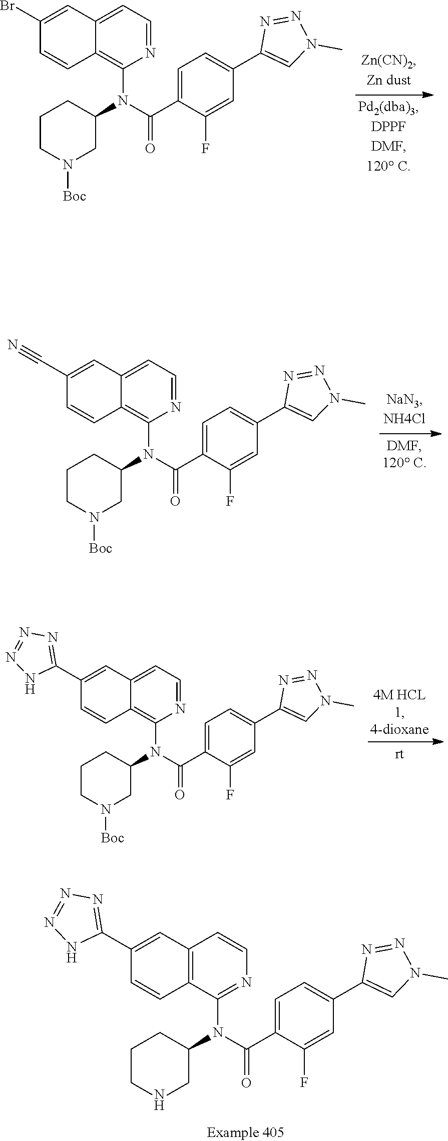 [SMILES: Cn1cc(-c2ccc(C(=O)N(c3nccc4cc(Br)ccc34)[C@@H]3CCCN(C(=O)OC(C)(C)C)C3)c(F)c2)nn1 ; Cn1cc(-c2ccc(C(=O)N(c3nccc4cc(C#N)ccc34)[C@@H]3CCCN(C(=O)OC(C)(C)C)C3)c(F)c2)nn1 ; Cn1cc(-c2ccc(C(=O)N(c3nccc4cc(-c5nnn[nH]5)ccc34)[C@@H]3CCCN(C(=O)OC(C)(C)C)C3)c(F)c2)nn1 ; Cn1cc(-c2ccc(C(=O)N(c3nccc4cc(-c5nnn[nH]5)ccc34)[C@@H]3CCCNC3)c(F)c2)nn1]

### Step 1:

To a stirred solution of tert-butyl (R)-3-(N-(6-bromoisoquinolin-1-yl)-2-fluoro-4-(1-methyl-1H-1,2,3-triazol-4-yl)benzamido)piperidine-1-carboxylate (160 mg, 0.263 mmol) in DMF (1 mL) was added zinc (II) cyanide (61.6 mg, 0.525 mmol), dppf (5.82 mg, 10.50 mmol) and zinc (3.43 mg, 0.053 mmol) and the reaction mixture was degassed with N₂ for 10 min. Then Pd₂(dba)₃ (9.62 mg, 10.50 mmol) was added to the reaction mixture and stirred at 120° C. for 2 h. The reaction mixture was cooled, diluted with water (10 mL), and extracted with EtOAc (2×50 mL). The combined organic layer was dried by Na₂SO₄ and concentrated under reduced pressure. The residue was purified by Combi-flash eluting with 50% EtOAc in Hexane to afford tert-butyl (R)-3-(N-(6-cyanoisoquinolin-1-yl)-2-fluoro-4-(1-methyl-1H-1,2,3-triazol-4-yl)benzamido)-piperidine-1-carboxylate (120 mg, 80%) as a light brown solid. LC-MS: [M+H-Boc]=502.05.

### Step 2:

To a solution of tert-butyl (R)-3-(N-(6-cyanoisoquinolin-1-yl)-2-fluoro-4-(1-methyl-1H-1,2,3-triazol-4-yl)benzamido)piperidine-1-carboxylate (110 mg, 0.198 mmol) in DMF (2 mL) was added sodium azide (25.7 mg, 0.396 mmol) and ammonium chloride (21.18 mg, 0.396 mmol) at room temperature and stirred at 120° C. in a sealed tube for 16 h. The reaction mixture was cooled, diluted with water (20 mL) and extracted with EtOAc (2×20 mL). The aqueous layer was extracted with 20% n-Butanol in DCM (3×20 mL). The organic layer was dried over Na₂SO₄ and concentrated under reduced pressure. The residue was purified by Prep-HPLC (Conditions: Kinetex evo C-18 (250×21.2×5 μm), Mobile phase A: 0.1% formic acid in water (100%), Mobile phase B: 100% MeCN, Gradient: Time (min)/% B: 0/10, 2/10, 10/75, Flow: 15 mL/min, Dilution: MeCN+THF). Pure fractions were combined and dried by lyophilization to afford tert-butyl (R)-3-(N-(6-(1H-tetrazol-5-yl)isoquinolin-1-yl)-2-fluoro-4-(1-methyl-1H-1,2,3-triazol-4-yl)-benzamido)piperidine-1-carboxylate (30 mg, 32%) as an off-white solid. LC-MS: [M+H⁺]=599.3.

### Step 3:

The title compound hydrochloride salt was obtained as an off-white solid by deprotection Method A used in Example 97. LC-MS: [M+H⁺]=499.2. ¹H NMR (CD₃OD, 400 MHZ): δ 8.59-8.50 (m, 2H), 8.40-8.30 (m, 2H), 8.13-8.12 (m, 1H), 7.90-7.88 (m, 1H), 7.27-7.17 (m, 2H), 7.12-7.08 (m, 1H), 5.32-5.24 (m, 1H), 4.02 (s, 3H), 3.96-3.94 (m, 1H), 3.74-3.68 (m, 1H), 2.90-3.83 (m, 1H), 2.04-2.01 (m, 1H), 1.93-1.81 (m, 2H), 1.40-1.17 (m, 2H).

### Synthesis of (R,E)-3-(1-(4-(3H-[1,2,3]triazolo[4,5-b]pyridin-3-yl)-2-fluoro-N-(piperidin-3-yl)benzamido)isoquinolin-6-yl)acrylic acid (Example 406)

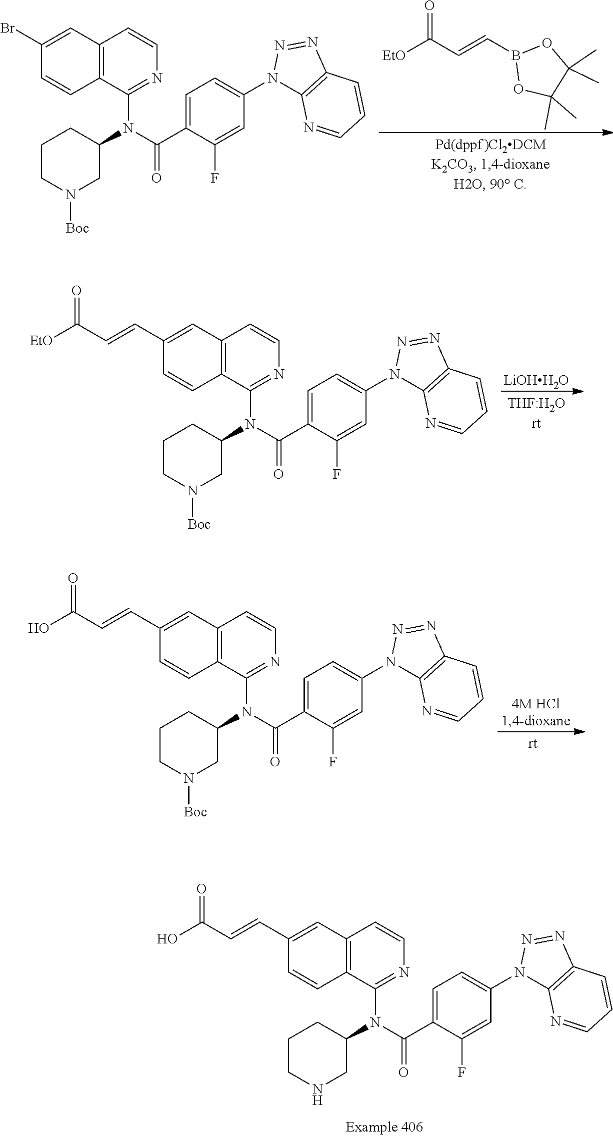

### Step 1:

To a stirred solution of tert-butyl (R)-3-(4-(3H-[1,2,3]triazolo[4,5-b]pyridin-3-yl)-N-(6-bromoisoquinolin-1-yl)-2-fluorobenzamido)piperidine-1-carboxylate (200 mg, 0.309 mmol) in 1,4-dioxane (2 mL) & H₂O (0.5 mL) was added ethyl (E)-3-(4,4,5,5-tetramethyl-1,3,2-dioxaborolan-2-yl)acrylate (140 mg, 0.619 mmol) and potassium carbonate (128 mg, 0.928 mmol) at room temperature. The reaction mixture was degassed with nitrogen for 5 mins. Then PdCl₂(dppf)-DCM (25.3 mg, 0.031 mmol) was added to the reaction mixture and stirred at 90° C. for 16 h in a sealed tube. The reaction mixture was diluted with EtOAc (50 mL) and filtered through celite. The filtrate was washed with water (30 mL). The organic layer was dried over Na₂SO₄ and concentrated. The residue was purified by Combi-flash using eluted with 15% EtOAc in hexane to afford tert-butyl (R,E)-3-(4-(3H-[1,2,3]triazolo[4,5-b]pyridin-3-yl)-N-(6-(3-ethoxy-3-oxoprop-1-en-1-yl) isoquinolin-1-yl)-2-fluorobenzamido) piperidine-1-carboxylate (120 mg, 43%) as an orange solid. LC-MS: [M+H⁺]=666.30.

### Step 2:

To a solution of tert-butyl (R,E)-3-(4-(3H-[1,2,3]triazolo[4,5-b]pyridin-3-yl)-N-(6-(3-ethoxy-3-oxoprop-1-en-1-yl)isoquinolin-1-yl)-2-fluorobenzamido)piperidine-1-carboxylate (50 mg, 0.075 mmol) in THF (1 mL) was added lithium hydroxide mono-hydrate (3.60 mg, 0.150 mmol) and H₂O (1 mL) at 0° C. and the mixture was then stirred at 25° C. for 16 h. The reaction mixture was diluted with water (20 mL), acidified with 1N aq. HCl to pH˜2 and the reaction mixture was extracted with EtOAc (50 mL). The organic layer was dried over Na₂SO₄ and concentrated under reduced pressure to provide crude material that was purified by prep HPLC (Conditions: Kinetex evo C-18 (250×21.2×5 μm), Mobile phase A: 0.1% formic acid in water (100%), Mobile phase B: 100% MeCN, Gradient: Time (min)/% B: 0/55, 10/80, Flow: 15 mL/min, Dilution: MeCN+THF. The pure fractions were combined and concentrated under reduced pressure to afford (R, E)-3-(1-(4-(3H-[1,2,3]triazolo[4,5-b] pyridin-3-yl)-N-(1-(tert-butoxycarbonyl)piperidin-3-yl)-2-fluorobenzamido) isoquinolin-6-yl)acrylic acid (30 mg, 29%) as an off-white solid. LC-MS: [M+H⁺]=638.30.

### Step 3:

The title compound hydrochloride salt was obtained as an off-white solid by deprotection Method A used in Example 97. LC-MS: [M−HCl+H⁺]=538.2. ¹H NMR (CD₃OD, 400 MHZ): δ 8.73 (d, J=4.4 Hz, 1H), 8.50-8.45 (m, 2H), 8.21-8.19 (m, 1H), 8.06-8.00 (m, 3H), 7.80-7.79 (m, 1H), 7.76-7.72 (m, 2H), 7.51-7.50 (m, 1H), 7.34-7.31 (m, 1H), 6.68 (d, J=16 Hz, 1H), 5.31-5.24 (m, 1H), 3.97-3.95 (m, 1H), 3.72-3.69 (m, 1H), 2.87-2.88 (m, 1H), 2.01-1.90 (m, 3H), 1.34-1.28 (m, 1H), 0.96-0.91 (m, 1H).

### Synthesis of ((R)-3-(1-(4-(3H-[1,2,3]triazolo[4,5-b]pyridin-3-yl)-2-fluoro-N-(piperidin-3-yl) benzamido)isoquinolin-6-yl)propanoic acid (Example 407)

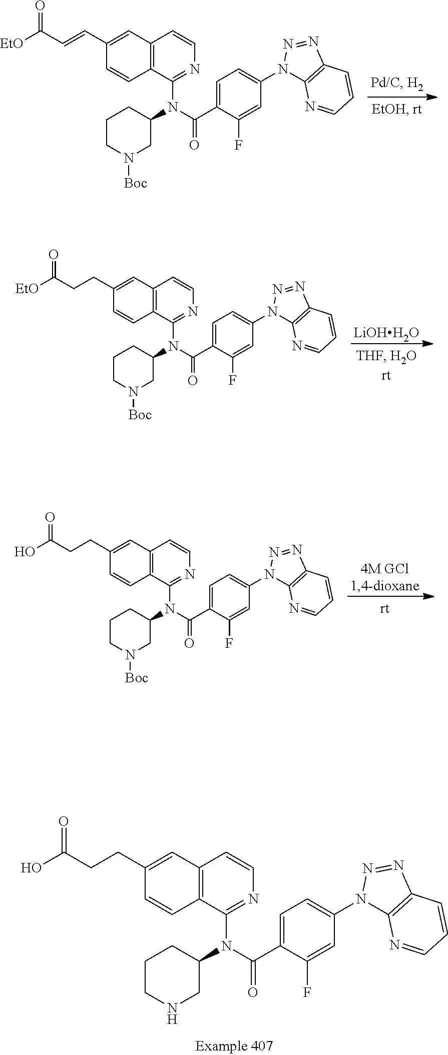

### Step 1:

To a solution of tert-butyl (R,E)-3-(4-(3H-[1,2,3]triazolo[4,5-b]pyridin-3-yl)-N-(6-(3-ethoxy-3-oxoprop-1-en-1-yl)isoquinolin-1-yl)-2-fluorobenzamido)piperidine-1-carboxylate (80 mg, 0.120 mmol) in Ethanol (5 mL) was added Pd/C (12.79 mg, 0.120 mmol) at room temperature and the reaction mixture was stirred under H₂ balloon for 5 h. The reaction mixture was filtered through celite bed, rinsed with DCM (50 mL). The filtrate was concentrated under reduced pressure to afford tert-butyl (R)-3-(4-(3H-[1,2,3]triazolo[4,5-b] pyridin-3-yl)-N-(6-(3-ethoxy-3-oxopropyl) isoquinolin-1-yl)-2-fluorobenzamido) piperidine-1-carboxylate (80 mg, 57%) as yellow solid. LC-MS: [M+H⁺]=668.3.

To a stirred solution of tert-butyl (R)-3-(4-(3H-[1,2,3]triazolo[4,5-b]pyridin-3-yl)-N-(6-(3-ethoxy-3-oxopropyl)isoquinolin-1-yl)-2-fluorobenzamido)piperidine-1-carboxylate (80 mg, 0.120 mmol) in THF (5 mL) was added lithium hydroxide (5.74 mg, 0.240 mmol) and H₂O (2 mL) at 0° C. The reaction mixture was stirred at room temperature for 16 hr. The reaction mixture was diluted with water (20 mL) and acidified with 1N HCl to pH 2 then the reaction mixture was dissolved in ethyl acetate (50 ml), washed with water (2×20 ml) and then the organic layer was dried by Na₂SO₄ concentrated under reduced pressure. The residue was purified by prep HPLC (Conditions: Luna OMEGA C-18 (250×21.2×5 μm), Mobile phase A: 0.1% formic acid in water (100%), Mobile phase B: 100% MeCN, Gradient: 0/50, 10/70, Flow: 15 mL/min, Dilution: MeCN+THF. The pure fractions were combined and concentrated under reduced pressure to afford (R)-3-(1-(4-(3H-[1,2,3] triazolo[4,5-b] pyridin-3-yl)-N-(1-(tert-butoxycarbonyl)piperidin-3-yl)-2-fluorobenzamido) isoquinolin-6-yl) propanoic acid (40 mg, 49%) as a yellow solid. LC-MS: [M+H⁺]=640.3.

### Step 3:

The title compound was obtained as a white solid by deprotection Method A used in Example 97. LC-MS: [M+H⁺]=540.2. ¹H NMR (CD₃OD, 400 MHz): δ 8.74 (d, J=4.4 Hz, 1H), 8.49 (d, J=8.4 Hz, 1H), 8.42-8.37 (m, 1H), 8.14-8.10 (m, 1H), 8.03-8.00 (m, 1H), 7.93-7.87 (m, 1H), 7.72-7.63 (m, 3H), 7.53-7.51 (m, 1H), 7.32-7.28 (m, 1H), 5.28-5.22 (m, 1H), 3.97-3.94 (m, 1H), 3.72-3.69 (m, 1H), 3.06-3.03 (m, 2H), 2.87-2.85 (m, 1H), 2.66-2.62 (m, 2H), 2.03-1.97 (m, 1H), 1.91-1.89 (m, 2H), 1.39-1.22 (m, 1H), 0.96-0.89 (m, 1H).

### Synthesis of (R)-2-fluoro-N-(8-methylisoquinolin-1-yl)-4-((4-morpholinopyrimidin-2-yl)amino)-N-(piperidin-3-yl)benzamide (Example 408)

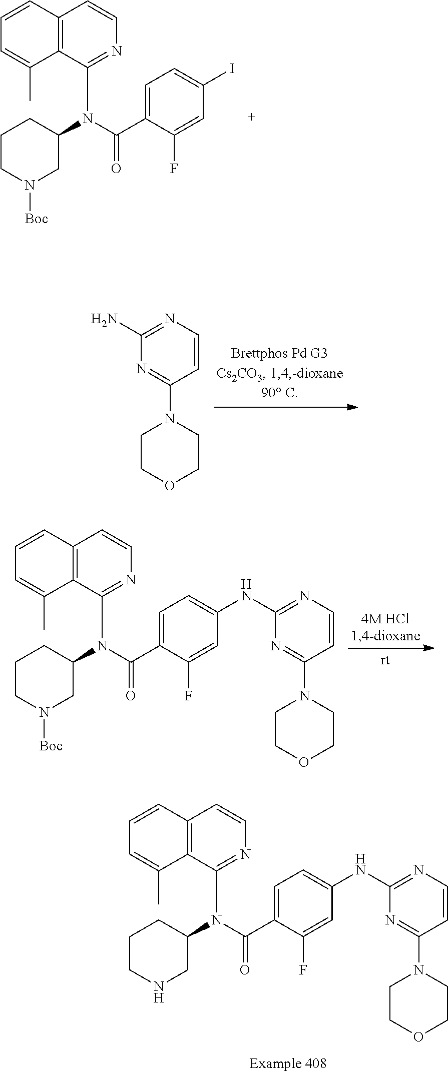 [SMILES: Cc1cccc2ccnc(N(C(=O)c3ccc(I)cc3F)[C@@H]3CCCN(C(=O)OC(C)(C)C)C3)c12 ; Nc1nccc(N2CCOCC2)n1 ; Cc1cccc2ccnc(N(C(=O)c3ccc(Nc4nccc(N5CCOCC5)n4)cc3F)[C@@H]3CCCN(C(=O)OC(C)(C)C)C3)c12 ; Cc1cccc2ccnc(N(C(=O)c3ccc(Nc4nccc(N5CCOCC5)n4)cc3F)[C@@H]3CCCNC3)c12]

Synthesis of Example 408 was prepared according to the procedure described for Example 131 by substituting the 3-amino-1-methylpyridine-2 (1H)-one with 4-morpholinopyrimidin-2-amine to afford the title compound hydrochloride salt as an off-white solid. LC-MS: [M+H⁺]=542.1. ¹H NMR (CD₃OD, 400 MHZ): δ 8.43-8.40 (m, 1H), 7.79-7.64 (m, 3H), 7.53-7.45 (m, 2H), 7.09-7.02 (m, 1H), 6.86-6.78 (m, 2H), 6.58 (d, J=7.6 Hz, 1H), 5.08-4.95 (m, 1H), 3.98-3.96 (m, 1H), 3.83-3.72 (m, 8H), 3.53-3.38 (m, 1H), 3.02-2.94 (m, 1H), 2.81 (s, 3H), 2.70-2.68 (m, 1H), 1.96-1.88 (m, 3H), 1.56-1.52 (m, 1H).

### Synthesis of (R)-2-fluoro-4-((5-fluoropyrimidin-2-yl)amino)-N-(8-methylisoquinolin-1-yl)-N-(piperidin-3-yl)benzamide (Example 409)

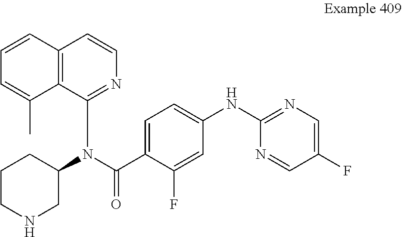 [SMILES: Cc1cccc2ccnc(N(C(=O)c3ccc(Nc4ncc(F)cn4)cc3F)[C@@H]3CCCNC3)c12]

Example 409 was prepared according to the procedure described for Example 408 by substituting 4-morpholinopyrimidin-2-amine with 5-fluoropyrimidin-2-amine, provided the title compound hydrochloride salt as an off-white solid. LC-MS: (M+1)=475.1. ¹H NMR (CD₃OD, 400 MHz): δ 8.51-8.43 (m, 1H), 8.36 (s, 2H), 7.74-7.70 (m, 1H), 7.67-7.62 (m, 1H), 7.51-7.47 (m, 1H), 7.45-7.42 (m, 2H), 6.81 (dd, J=2.0 Hz, J=8.4 Hz, 1H), 6.63-6.59 (m, 1H), 5.09-5.02 (m, 1H), 3.95-3.93 (m, 1H), 3.84-3.78 (m, 1H), 3.40-3.32 (m, 1H), 3.03-2.95 (m, 1H), 2.79 (s, 3H), 1.94-1.89 (m, 3H), 1.59-1.55 (m, 1H).

### Synthesis of (R)-4-((5-aminopyrimidin-2-yl)amino)-2-fluoro-N-(8-methylisoquinolin-1-yl)-N-(piperidin-3-yl)benzamide (Example 410)

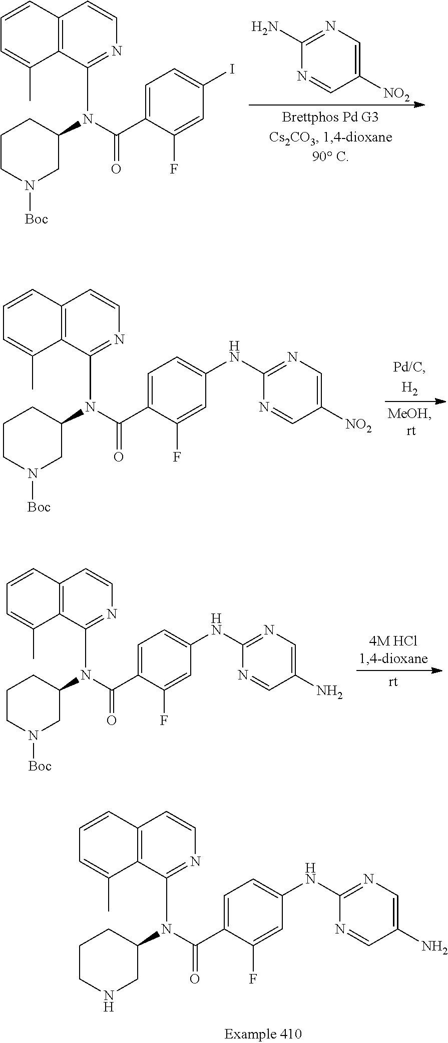 [SMILES: Cc1cccc2ccnc(N(C(=O)c3ccc(I)cc3F)[C@@H]3CCCN(C(=O)OC(C)(C)C)C3)c12 ; Nc1ncc([N+](=O)[O-])cn1 ; Cc1cccc2ccnc(N(C(=O)c3ccc(Nc4ncc([N+](=O)[O-])cn4)cc3F)[C@@H]3CCCN(C(=O)OC(C)(C)C)C3)c12 ; Cc1cccc2ccnc(N(C(=O)c3ccc(Nc4ncc(N)cn4)cc3F)[C@@H]3CCCN(C(=O)OC(C)(C)C)C3)c12 ; Cc1cccc2ccnc(N(C(=O)c3ccc(Nc4ncc(N)cn4)cc3F)[C@@H]3CCCNC3)c12]

### Step 1:

To a solution of tert-butyl (R)-3-(2-fluoro-4-iodo-N-(8-methylisoquinolin-1-yl)benzamido)piperidine-1-carboxylate (130 mg, 0.221 mmol) and 5-nitropyrimidin-2-amine (30.9 mg, 0.221 mmol) in 1,4-dioxane (3 mL) was added Cs₂CO₃ (144 mg, 0.441 mmol) at room temperature and the reaction mixture was degassed with nitrogen for 5 min. Then, Brett Phos PdG-3 (19.99 mg, 0.022 mmol) was added to reaction mixture and stirred at 100° C. for 16 h. The reaction was cooled to room temperature and diluted with EtOAc. The organic layer was dried over Na₂SO₄ and concentrated under reduced pressure. The obtained crude compound was purified by combi-flash using eluted with 30% EtOAc in Hexane to affords tert-butyl (R)-3-(2-fluoro-N-(8-methylisoquinolin-1-yl)-4-((5-nitropyrimidin-2-yl) amino) benzamido) piperidine-1-carboxylate (120 mg, 38%) as an off-white sold. LC-MS: [M−H]=600.2.

### Step 2:

To a Solution of tert-butyl (R)-3-(2-fluoro-N-(8-methylisoquinolin-1-yl)-4-((5-nitropyrimidin-2-yl) amino) benzamido) piperidine-1-carboxylate (110 mg, 0.183 mmol) in MeOH (5 mL) and THF (2 mL) was added Pd—C(20 mg, 0.019 mmol) at room temperature and the reaction mixture was stirred under H₂ balloon at room temperature for 24 h. The reaction mixture was filtered through celite pad and filtrate was concentrated under reduced pressure to afford (65 mg) as pale-yellow solid. The residue was purified by Prep-HPLC (Conditions: Kinetex evo C-18 (250×21.2×5 μm)), Mobile phase A: 0.1% formic acid in water (100%), Mobile phase B: 100% MeCN, Gradient: T/% B: 0/55, 10/70 Flow: 15 mL/min, Dilution: MeCN+THF. The pure fractions were concentrated under reduced pressure to affords tert-butyl (R)-3-(4-((5-aminopyrimidin-2-yl)amino)-2-fluoro-N-(8-methylisoquinolin-1-yl)benzamido)piperidine-1-carboxylate (14 g, 20%) as yellow solid. LC-MS: [M+H⁺]=572.3.

### Step 3:

The title compound hydrochloride salt was obtained as an off-white solid by deprotection Method A used in Example 97. LC-MS: [M−HCl+H⁺]=472.25. ¹H NMR (CD₃OD, 400 MHZ): δ 8.43 (s, 2H), 8.42-8.40 (m, 1H), 7.74-7.63 (m, 2H), 7.52-7.43 (m, 3H), 6.83 (dd, J=2 Hz, J=8.4 Hz, 1H), 6.68-6.64 (m, 1H), 5.08-5.06 (m, 1H), 3.97-3.94 (m, 1H), 3.50-3.40 (m, 1H), 3.03-2.99 (m, 1H), 2.80 (s, 3H), 1.98-1.89 (m, 3H), 1.58-1.42 (m, 1H), 0.96-0.91 (m, 1H).

### Synthesis of 2-fluoro-N-[(3R)-3-piperidyl]-N-[2-[2-(4-pyridyl)ethynyl]thieno[3,2-c]pyridin-4-yl]-4-(triazolo[4,5-b]pyridin-3-yl)benzamide (Example 411)

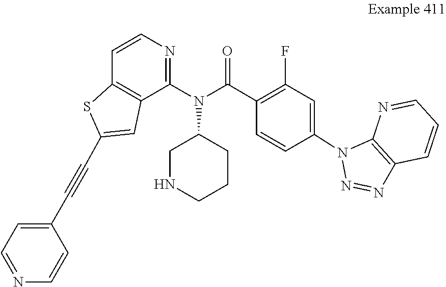 [SMILES: O=C(c1ccc(-n2nnc3cccnc32)cc1F)N(c1nccc2sc(C#Cc3ccncc3)cc12)[C@@H]1CCCNC1]

Example 411 was prepared according to the procedure described for Example 376 by substituting 2-methylbut-3-yn-2-ol with 4-ethynylpyridine, provided the title compound as an off-white solid. LC-MS: [M+H⁺]=575.3. ¹H NMR (400 MHZ, CD₃OD): δ 8.76 (br s, 1H), 8.62 (d, J=5.9 Hz, 2H), 8.51 (br d, J=8.0 Hz, 1H), 8.41 (br s, 1H), 8.16-7.95 (m, 2H), 7.87 (br s, 2H), 7.60 (d, J=5.8 Hz, 2H), 7.57-7.36 (m, 2H), 3.68-3.42 (m, 1H), 3.22-3.01 (m, 1H), 3.00-2.86 (m, 1H), 2.60-2.28 (m, 2H), 2.12-1.92 (m, 1H), 1.72 (br s, 2H), 1.41-1.17 (m, 1H).

### Synthesis of N-[2-(4-aminophenyl)thieno[3,2-c]pyridin-4-yl]-2-fluoro-N-[(3R)-3-piperidyl]-4-(triazolo[4,5-b]pyridin-3-yl)benzamide (Example 412)

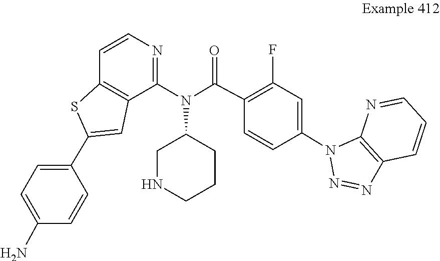 [SMILES: Nc1ccc(-c2cc3c(N(C(=O)c4ccc(-n5nnc6cccnc65)cc4F)[C@@H]4CCCNC4)nccc3s2)cc1]

Example 412 was prepared according to the procedure described for Example 161 by substituting 4-pyridylboronic acid with 4-(4,4,5,5-tetramethyl-1,3,2-dioxaborolan-2-yl)aniline, provided the title compound hydrochloride salt as an off-white solid. LC-MS: [M+H⁺]=565.3. ¹H NMR (400 MHZ, DMSO-d₆): δ 9.25-8.90 (m, 2H), 8.82-8.77 (m, 1H), 8.67 (d, J=7.8 Hz, 1H), 8.31-8.24 (m, 1H), 8.00 (br d, J=10.9 Hz, 1H), 7.93 (d, J=5.3 Hz, 1H), 7.88 (br d, J=8.3 Hz, 1H), 7.79-7.31 (m, 7H), 6.83 (br s, 2H), 4.95 (br s, 1H), 3.74 (br s, 1H), 3.19 (br d, J=10.5 Hz, 1H), 2.80-2.67 (m, 1H), 2.06 (br d, J=9.5 Hz, 1H), 1.94-1.68 (m, 3H), 1.25-1.03 (m, 1H).

### Synthesis of 2-hydroxyethyl 4-[4-[[2-fluoro-4-(triazolo[4,5-b]pyridin-3-yl)benzoyl]-[(3R)-3-piperidyl]amino]thieno[3,2-c]pyridin-2-yl]benzoate (Example 413)

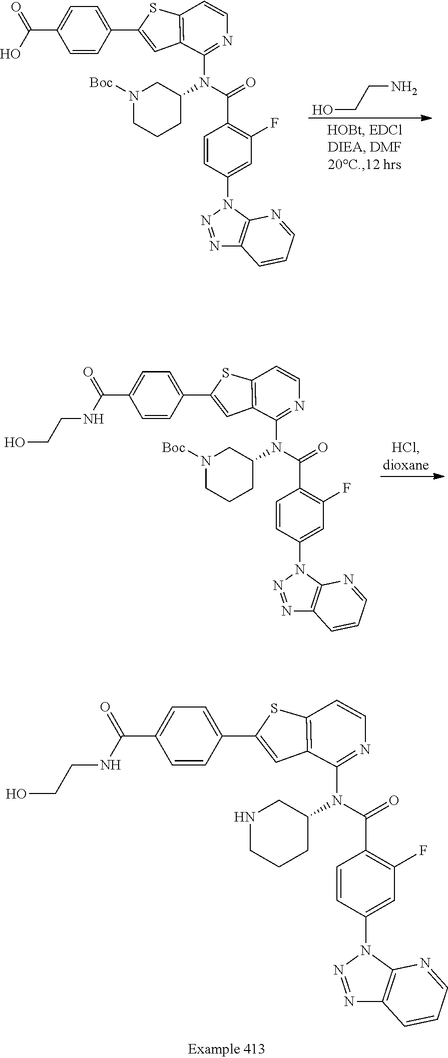

### Step 1:

To a solution of 4-[4-[[(3R)-1-tert-butoxycarbonyl-3-piperidyl]-[2-fluoro-4-(triazolo[4,5-b] pyridin-3-yl)benzoyl]amino]thieno[3,2-c]pyridin-2-yl]benzoic acid (50 mg, 72.07 μmol) in DCM (2 mL) was added HOBt (14.61 mg, 108.11 μmol) and EDCI (20.72 mg, 108.11 μmol) at 20° C., then the mixture was stirred at 20° C. for 5 min, then DIEA (18.63 mg, 144.15 μmol) and 2-aminoethan-1-ol (6.60 mg, 108.11 μmol) was added at 20° C., then the mixture was stirred at 20° C. for 12 h. The reaction was poured into water and extracted with EtOAc (10 mL). The organic layer was washed with brine, dried with anhydrous sodium sulfate, and concentrated to give tert-butyl (3R)-3-[[2-fluoro-4-(triazolo[4,5-b]pyridin-3-yl)benzoyl]-[2-[4-(2-hydroxyethylcarbamoyl)phenyl]thieno[3,2-c]pyridin-4-yl]amino]piperidine-1-carboxylate (50 mg, 94%) as a yellow solid. LC-MS: [M+H]=737.4.

### Step 2:

The title compound hydrochloride salt was obtained as a white solid by deprotection Method A used in Example 97. LC-MS: [M+H]=637.3. ¹H NMR (400 MHZ, DMSO-d₆): δ 9.01-8.73 (m, 1H), 8.71-8.54 (m, 2H), 8.52-8.30 (m, 1H), 8.25-8.10 (m, 1H), 8.08-7.89 (m, 6H), 7.88-7.65 (m, 1H), 7.64-7.38 (m, 2H), 4.84-4.75 (m, 1H), 4.75-4.60 (m, 1H), 3.54 (q, J=6.0 Hz, 2H), 3.40-3.37 (m, 2H), 2.96-2.84 (m, 1H), 2.82-2.71 (m, 1H), 2.28-2.08 (m, 2H), 2.07-1.91 (m, 1H), 1.84-1.35 (m, 3H), 1.27-0.75 (m, 1H). ¹⁹F NMR (376 MHz, DMSO-d₆): δ −111.17 (br s, 1F).

### Synthesis of 2-fluoro-N-[(3R)-3-piperidyl]-N-[2-(4-sulfamoylphenyl)thieno[3,2-c]pyridin-4-yl]-4-(triazolo[4,5-b]pyridin-3-yl)benzamide (Example 414)

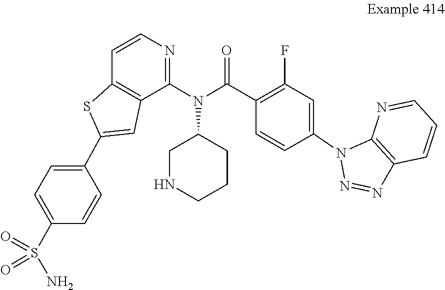 [SMILES: NS(=O)(=O)c1ccc(-c2cc3c(N(C(=O)c4ccc(-n5nnc6cccnc65)cc4F)[C@@H]4CCCNC4)nccc3s2)cc1]

Example 414 was prepared according to the procedure described for Example 161 by substituting 4-pyridylboronic acid with (4-sulfamoylphenyl)boronic acid, provided the title compound hydrochloride salt as an off-white solid. LC-MS: [M+H]=629.3. ¹H NMR (400 MHz, DMSO-d₆): δ 8.79 (br d, J=4.3 Hz, 1H), 8.66 (br d, J=8.0 Hz, 1H), 8.41-8.33 (m, 1H), 8.19 (br s, 1H), 8.13-7.91 (m, 7H), 7.87-7.80 (m, 1H), 7.58 (br dd, J=4.7, 8.4 Hz, 1H), 7.49 (br s, 3H), 4.79-4.60 (m, 1H), 2.95-2.74 (m, 1H), 2.17-1.95 (m, 3H), 1.69-1.41 (m, 3H), 1.06-0.91 (m, 1H).

### Synthesis of 2-fluoro-N-[2-(1-isobutyltriazol-4-yl)thieno[3,2-c]pyridin-4-yl]-N-[(3R)-3-piperidyl]-4-(triazolo[4,5-b]pyridin-3-yl)benzamide (Example 415)

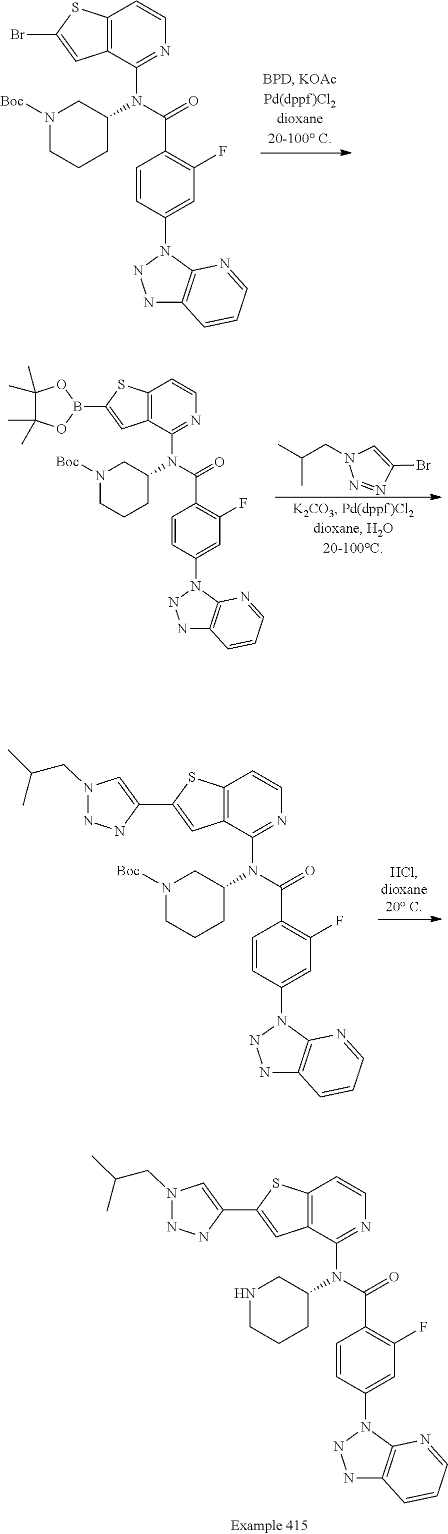

### Step 1:

A mixture of tert-butyl (3R)-3-[(2-bromothieno[3,2-c]pyridin-4-yl)-[2-fluoro-4-(triazolo[4,5-b]pyridin-3-yl)benzoyl]amino]piperidine-1-carboxylate (200.00 mg, 275.85 μmol), BPD (210.14 mg, 827.54 μmol), KOAc (54.14 mg, 551.69 μmol) and Pd(dppf)Cl₂ (20.18 mg, 27.58 μmol) in dioxane (3 mL) was purged with N₂, and then the mixture was stirred at 100° C. for 2 hours under N₂ atmosphere. The reaction mixture was concentrated to give crude tert-butyl (3R)-3-[[2-fluoro-4-(triazolo[4,5-b]pyridin-3-yl)benzoyl]-[2-(4,4,5,5-tetramethyl-1,3,2-dioxaborolan-2-yl)thieno[3,2-c]pyridin-4-yl]amino]piperidine-1-carboxylate (190 mg) as a brown solid.

### Step 2:

A mixture of 4-bromo-1-isobutyl-triazole (49.26 mg, 217.27 μmol), tert-butyl (3R)-3-[[2-fluoro-4-(triazolo[4,5-b]pyridin-3-yl)benzoyl]-[2-(4,4,5,5-tetramethyl-1,3,2-dioxaborolan-2-yl)thieno[3,2-c]pyridin-4-yl]amino]piperidine-1-carboxylate (190 mg, 271.58 μmol), K₂CO₃ (75.07 mg, 543.17 μmol), Pd(dppf)Cl₂ (19.87 mg, 27.16 μmol) in dioxane (1.8 mL) and H₂O (0.2 mL) was purged with N₂, and then the mixture was stirred at 100° C. for 2 h under N₂ atmosphere. The reaction mixture was filtered, and the filtrate was concentrated. The residue was purified by Prep-HPLC (column: Phenomenex luna C18 100*40 mm*5 μm; mobile phase: [H₂O (0.04% HCl)-MeCN]; gradient: 60%-85% B over 8.0 min) and the pure fraction was lyophilized to afford tert-butyl (3R)-3-[[2-fluoro-4-(triazolo[4,5-b]pyridin-3-yl)benzoyl]-[2-(1-isobutyltriazol-4-yl)thieno[3,2-c]pyridin-4-yl]amino]piperidine-1-carboxylate (5 mg, 2%) as a white solid.

### Step 3:

The title compound hydrochloride salt was obtained as an off-white solid by deprotection Method A used in Example 97. LC-MS: [M+H⁺]=597.4. ¹H NMR (400 MHZ, DMSO-d₆): δ 8.87 (s, 2H), 8.80 (br d, J=4.4 Hz, 1H), 8.70-8.64 (m, 1H), 8.38 (br d, J=5.1 Hz, 1H), 8.04 (br d, J=5.3 Hz, 1H), 8.02-7.95 (m, 2H), 7.89 (br d, J=9.0 Hz, 1H), 7.60 (dd, J=4.6, 8.4 Hz, 1H), 7.55-7.49 (m, 1H), 5.21-5.07 (m, 1H), 4.32 (br d, J=7.4 Hz, 2H), 3.79-3.72 (m, 1H), 3.20 (td, J=1.7, 10.6 Hz, 1H), 2.77-2.71 (m, 1H), 2.28-2.08 (m, 1H), 1.76-1.74 (m, 1H), 2.08-1.70 (m, 3H), 1.27-1.18 (m, 2H), 0.94 (dd, J=4.0, 6.4 Hz, 6H).

### Synthesis of 2-fluoro-N-[(3R)-3-piperidyl]-N-(2-thiazol-4-ylthieno[3,2-c]pyridin-4-yl)-4-(triazolo[4,5-b]pyridin-3-yl)benzamide (Example 416)

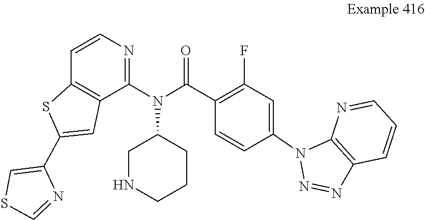 [SMILES: O=C(c1ccc(-n2nnc3cccnc32)cc1F)N(c1nccc2sc(-c3cscn3)cc12)[C@@H]1CCCNC1]

Example 416 was prepared according to the procedure described for Example 161 by substituting tributyl(thiazol-2-yl)stannane with tributyl(thiazol-4-yl)stannane, provided the title compound hydrochloride salt (65%) as off white solid. LC-MS: [M+H]=557.3. ¹H NMR (400 MHZ, DMSO-d₆): δ 9.28-9.20 (m, 1H), 8.91 (br dd, J=8.7, 9.8 Hz, 1H), 8.80 (br d, J=3.6 Hz, 2H), 8.67 (br d, J=7.8 Hz, 1H), 8.57 (s, 1H), 8.40-8.34 (m, 1H), 8.22-8.17 (m, 1H), 8.05-7.95 (m, 2H), 7.92-7.84 (m, 1H), 7.62-7.48 (m, 2H), 5.19-4.90 (m, 1H), 3.81-3.68 (m, 1H), 3.24-3.16 (m, 2H), 2.80-2.69 (m, 1H), 2.13-2.01 (m, 1H), 1.97-1.86 (m, 1H), 1.85-1.69 (m, 2H), 1.63-1.55 (m, 1H).

### Synthesis of 2-fluoro-N-[(3R)-3-piperidyl]-N-(2-thiazol-2-ylthieno[3,2-c]pyridin-4-yl)-4-(triazolo[4,5-b]pyridin-3-yl)benzamide (Example 417)

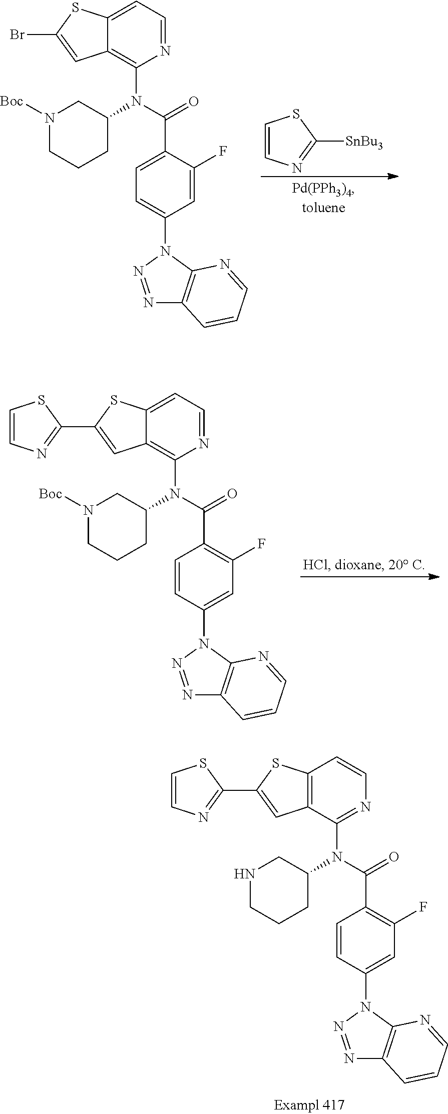

### Step 1:

A mixture of tributyl(thiazol-2-yl)stannane (154.82 mg, 413.77 μmol, 3 eq), tert-butyl (3R)-3-[(2-bromothieno[3,2-c]pyridin-4-yl)-[2-fluoro-4-(triazolo[4,5-b]pyridin-3-yl)benzoyl]amino]piperidine-1-carboxylate (100 mg, 137.92 μmol), Pd(PPh₃)₄ (47.81 mg, 41.38 μmol) in toluene (2 mL) was degassed and purged with N₂, and then the mixture was stirred at 100° C. for 3 h under N₂ atmosphere. The reaction mixture was filtered and the filtrate was concentrated to give crude tert-butyl (3R)-3-[[2-fluoro-4-(triazolo[4,5-b]pyridin-3-yl)benzoyl]-(2-thiazol-2-ylthieno[3,2-c]pyridin-4-yl)amino]piperidine-1-carboxylate (150 mg) as a yellow oil, which was used in next step directly without purification.

### Step 2:

Example 417 was prepared according to the procedure described for Example 161 provided the title compound hydrochloride salt as off white solid. LC-MS: [M+H⁺]=557.3. ¹H NMR (400 MHZ, DMSO-d₆): δ 9.10-8.86 (m, 2H), 8.80 (br d, J=3.8 Hz, 1H), 8.70-8.64 (m, 1H), 8.44 (br d, J=5.5 Hz, 1H), 8.29-8.23 (m, 1H), 8.06 (br d, J=5.3 Hz, 1H), 8.02-7.94 (m, 3H), 7.89 (br d, J=7.9 Hz, 1H), 7.59 (br dd, J=4.4, 8.1 Hz, 1H), 7.51-7.43 (m, 1H), 5.21-4.94 (m, 1H), 3.76 (br dd, J=2.0, 9.5 Hz, 1H), 3.43-3.40 (m, 1H), 3.20 (br d, J=10.8 Hz, 1H), 2.76-2.69 (m, 1H), 2.12-1.68 (m, 4H), 1.22-1.04 (m, 1H).

### Synthesis of (R)-4-(3H-[1,2,3]triazolo[4,5-b]pyridin-3-yl)-2-fluoro-N-(2-(1-(2-hydroxyethyl)-1H-pyrazol-3-yl)thieno[3,2-c]pyridin-4-yl)-N-(piperidin-3-yl)benzamide (Example 418)

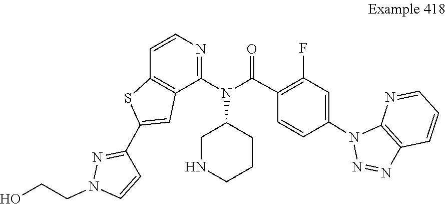 [SMILES: O=C(c1ccc(-n2nnc3cccnc32)cc1F)N(c1nccc2sc(-c3ccn(CCO)n3)cc12)[C@@H]1CCCNC1]

Example 415 was prepared according to the procedure described for Example 161 by substituting 4-pyridylboronic acid with [1-[2-[tert-butyl (dimethyl) silyl] oxyethyl] pyrazol-3-yl] boronic acid, provided the title compound hydrochloride salt as an off-white solid. LC-MS: [M+H⁺]=584.4. ¹H NMR (400 MHZ, CD₃OD): δ 8.74 (br s, 1H), 8.54-8.42 (m, 1H), 8.35 (br s, 1H), 8.16-7.82 (m, 3H), 7.82-7.70 (m, 2H), 7.62-7.36 (m, 2H), 6.83 (br s, 1H), 5.24 (br s, 1H), 4.27 (br t, J=4.8 Hz, 2H), 3.92 (br t, J=4.9 Hz, 3H), 3.82-3.54 (m, 1H), 3.43-3.33 (m, 1H), 2.89 (br s, 1H), 2.20-2.07 (m, 1H), 2.07-1.84 (m, 2H), 1.58-1.20 (m, 1H).

### Synthesis of 2-fluoro-N-[2-(3-methylsulfonylphenyl)thieno[3,2-c]pyridin-4-yl]-N-[(3R)-3-piperidyl]-4-(triazolo[4,5-b]pyridin-3-yl)benzamide (Example 419)

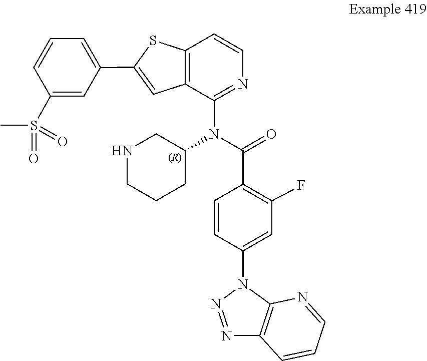

Example 419 was prepared according to the procedure described for Example 161 by substituting 4-pyridylboronic acid with (3-methylsulfonylphenyl) boronic acid, provided the title compound hydrochloride salt as off white solid. LC-MS: [M+H⁺]=628.3. ¹H NMR (400 MHz, DMSO-d₆): δ 9.11-8.86 (m, 2H), 8.79 (d, J=4.0 Hz, 1H), 8.71-8.64 (m, 1H), 8.44-8.32 (m, 3H), 8.21 (br d, J=7.6 Hz, 1H), 8.08 (d, J=5.5 Hz, 1H), 8.05-7.96 (m, 2H), 7.90-7.78 (m, 2H), 7.62-7.50 (m, 2H), 5.23-4.96 (m, 1H), 3.87-3.70 (m, 1H), 3.32 (br s, 3H), 3.22-3.16 (m, 1H), 2.72 (br d, J=9.5 Hz, 1H), 2.18-2.06 (m, 1H), 1.97-1.67 (m, 3H), 1.19-1.04 (m, 1H).

### Synthesis of N-[2-(3-amino-1,2-benzoxazol-5-yl)thieno[3,2-c]pyridin-4-yl]-2-fluoro-N-[(3R)-3-piperidyl]-4-(triazolo[4,5-b]pyridin-3-yl)benzamide (Example 420)

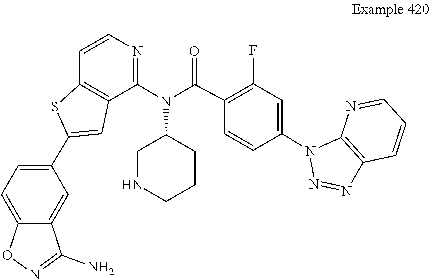 [SMILES: Nc1noc2ccc(-c3cc4c(N(C(=O)c5ccc(-n6nnc7cccnc76)cc5F)[C@@H]5CCCNC5)nccc4s3)cc12]

Example 420 was prepared according to the procedure described for Example 161 by substituting 4-pyridylboronic acid with tert-butyl N-tert-butoxycarbonyl-N-[5-(4,4,5,5-tetramethyl-1,3,2-dioxaborolan-2-yl)-1,2-benzoxazol-3-yl] carbamate, provided the title compound hydrochloride salt as an off-white solid. LC-MS: [M+H⁺]=606.3. ¹H NMR (400 MHZ, CD₃OD): δ 8.73 (br d, J=3.6 Hz, 1H), 8.50 (br d, J=8.3 Hz, 1H), 8.38 (br d, J=5.5 Hz, 1H), 8.19 (s, 1H), 8.11 (br d, J=10.5 Hz, 1H), 8.07-7.97 (m, 2H), 7.89 (d, J=5.5 Hz, 1H), 7.82-7.74 (m, 1H), 7.59-7.42 (m, 3H), 5.28 (br t, J=12.0 Hz, 1H), 3.98-3.85 (m, 1H), 3.67 (br t, J=11.9 Hz, 1H), 3.35 (br s, 1H), 2.89 (dt, J=4.0, 12.4 Hz, 1H), 2.20-2.08 (m, 1H), 2.03-1.85 (m, 2H), 1.50-1.35 (m, 1H).

### Synthesis of (R)-4-(3H-[1,2,3]triazolo[4,5-b]pyridin-3-yl)-2-fluoro-N-(8-methyl-6-(pyridin-3-yl)isoquinolin-1-yl)-N-(piperidin-3-yl)benzamide (Example 421)

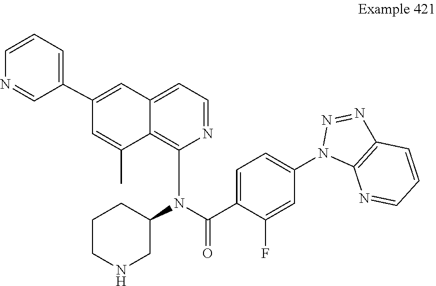 [SMILES: Cc1cc(-c2cccnc2)cc2ccnc(N(C(=O)c3ccc(-n4nnc5cccnc54)cc3F)[C@@H]3CCCNC3)c12]

Example 421 was prepared according to the procedure described for Example 97 by substituting tert-butyl (R)-3-(4-(3H-[1,2,3]triazolo[4,5-b]pyridin-3-yl)-N-(6-bromoisoquinolin-1-yl)-2-fluorobenzamido)piperidine-1-carboxylate and (1H-pyrazol-4-yl)boronic acid with tert-butyl (R)-3-(4-(3H-[1,2,3]triazolo[4,5-b]pyridin-3-yl)-N-(6-bromo-8-methylisoquinolin-1-yl)-2-fluorobenzamido)piperidine-1-carboxylate and pyridin-3-yl boronic acid, provided the title compound hydrochloride salt as an off-white solid. LC-MS: [M+H⁺]=559.2. ¹H NMR (CD₃OD, 400 MHZ): δ 9.27 (s, 1H), 9.00-8.96 (m, 1H), 8.88-8.86 (m, 1H), 8.73-8.72 (m, 1H), 8.61-8.56 (m, 1H), 8.50-8.48 (m, 1H), 8.18-8.16 (m, 2H), 7.99-7.95 (m, 3H), 7.92-7.85 (m, 1H), 7.54-7.51 (m, 1H), 7.18-7.12 (m, 1H), 5.19-5.16 (m, 1H), 4.22-4.03 (m, 1H), 3.91-3.73 (m, 1H), 3.48-3.42 (m, 1H), 3.00 (s, 3H), 2.00-1.97 (m, 2H), 1.79-1.68 (m, 1H), 1.43-1.28 (m, 1H), 0.96-0.89 (m, 1H).

### Synthesis of (R)-4-(3H-[1,2,3]triazolo[4,5-b]pyridin-3-yl)-2-fluoro-N-(8-methyl-6-(1H-pyrazol-4-yl)isoquinolin-1-yl)-N-(piperidin-3-yl)benzamide (Example 422)

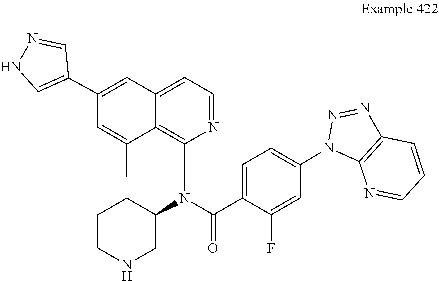 [SMILES: Cc1cc(-c2cn[nH]c2)cc2ccnc(N(C(=O)c3ccc(-n4nnc5cccnc54)cc3F)[C@@H]3CCCNC3)c12]

Example 422 was prepared according to the procedure described for Example 97 by substituting tert-butyl (R)-3-(4-(3H-[1,2,3]triazolo[4,5-b]pyridin-3-yl)-N-(6-bromoisoquinolin-1-yl)-2-fluorobenzamido)piperidine-1-carboxylate and (1H-pyrazol-4-yl)boronic acid with tert-butyl (R)-3-(4-(3H-[1,2,3]triazolo[4,5-b]pyridin-3-yl)-N-(6-bromo-8-methylisoquinolin-1-yl)-2-fluorobenzamido)piperidine-1-carboxylate and (1H-pyrazol-4-yl)boronic acid, provided the title compound hydrochloride salt as an off-white solid. LC-MS: [M+H⁺]=548.2. ¹H NMR (CD₃OD, 400 MHZ): δ 8.71-8.70 (m, 1H), 8.47 (d, J=8.4 Hz, 1H), 8.42-8.39 (m, 1H), 8.14-8.12 (m, 2H), 8.05-8.01 (m, 1H), 7.87-7.85 (m, 2H), 7.81-7.79 (m, 1H), 7.73-7.68 (m, 1H), 7.52-7.49 (m, 1H), 7.17-7.13 (m, 1H), 5.11-5.05 (m, 1H), 4.22-4.02 (m, 1H), 3.86-3.64 (m, 1H), 3.47-3.39 (m, 1H), 3.12-3.09 (m, 1H), 2.90 (s, 3H), 1.98-1.94 (m, 2H), 1.36-1.28 (m, 1H), 0.96-0.84 (m, 1H).

### Synthesis of 2-fluoro-N-[2-(1-isopropylpyrazol-4-yl)thieno[3,2-c]pyridin-4-yl]-N-[(3R)-3-piperidyl]-4-(triazolo[4,5-b]pyridin-3-yl)benzamide (Example 423)

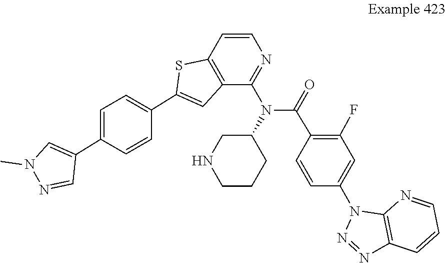 [SMILES: Cn1cc(-c2ccc(-c3cc4c(N(C(=O)c5ccc(-n6nnc7cccnc76)cc5F)[C@@H]5CCCNC5)nccc4s3)cc2)cn1]

Example 423 was prepared according to the procedure described for Example 161 by substituting 4-pyridylboronic acid with 1-methyl-4-(4,4,5,5-tetramethyl-1,3,2-dioxaborolan-2-yl)-1H-pyrazole, provided the title compound hydrochloride salt as a white solid. LC-MS: [M+H]=554.4. ¹H NMR (400 MHZ, DMSO-d₆): δ 9.12 (br d, J=10.1 Hz, 2H), 8.81 (d, J=3.5 Hz, 1H), 8.68 (d, J=8.1 Hz, 1H), 8.37-8.24 (m, 2H), 8.04-7.94 (m, 2H), 7.88 (br d, J=8.4 Hz, 1H), 7.73 (s, 1H), 7.64-7.47 (m, 2H), 5.24-4.86 (m, 1H), 3.90 (s, 3H), 3.79-3.66 (m, 1H), 3.35-3.26 (m, 1H), 3.19 (br d, J=11.0 Hz, 1H), 2.88-2.69 (m, 1H), 2.30-1.99 (m, 1H), 1.95-1.67 (m, 2H), 1.27-1.06 (m, 1H); ¹⁹F NMR (376 MHz, DMSO-d₆): δ −111.00 (s, 1F).

### Synthesis of 2-fluoro-N-[2-[3-(hydroxymethyl)phenyl]thieno[3,2-c]pyridin-4-yl]-N-[(3R)-3-piperidyl]-4-(triazolo[4,5-b]pyridin-3-yl)benzamide (Example 424)

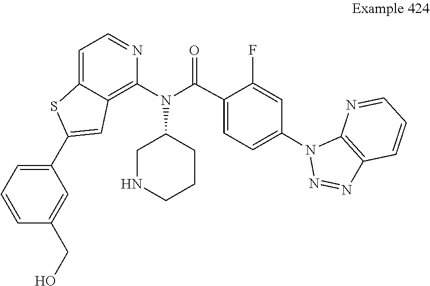 [SMILES: O=C(c1ccc(-n2nnc3cccnc32)cc1F)N(c1nccc2sc(-c3cccc(CO)c3)cc12)[C@@H]1CCCNC1]

Example 424 was prepared according to the procedure described for Example 161 by substituting 4-pyridylboronic acid with [3-(hydroxymethyl)phenyl]boronic acid, provided the title compound as an off-white solid. LC-MS: [M+H⁺]=580.4. ¹H NMR (400 MHZ, DMSO-d₆): δ 8.79 (br d, J=4.0 Hz, 1H), 8.66 (br d, J=8.3 Hz, 1H), 8.39-8.28 (m, 1H), 8.03-7.92 (m, 3H), 7.88-7.79 (m, 2H), 7.74 (br d, J=7.0 Hz, 1H), 7.58 (br dd, J=4.7, 8.1 Hz, 1H), 7.53-7.38 (m, 3H), 5.35 (br t, J=5.6 Hz, 1H), 4.78-4.64 (m, 1H), 4.60 (d, J=5.6 Hz, 2H), 3.29-3.20 (m, 1H), 2.95-2.69 (m, 2H), 2.27-2.15 (m, 2H), 2.05-1.94 (m, 1H), 1.80-1.41 (m, 3H).

### Synthesis of N-[2-(5-cyano-2-thienyl)thieno[3,2-c]pyridin-4-yl]-2-fluoro-N-[(3R)-3-piperidyl]-4-(triazolo[4,5-b]pyridin-3-yl)benzamide (Example 425)

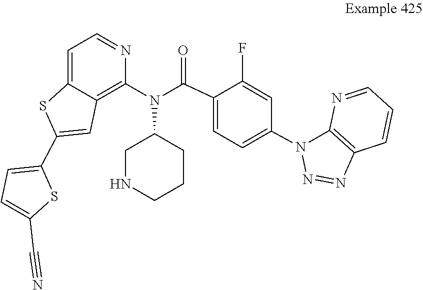 [SMILES: N#Cc1ccc(-c2cc3c(N(C(=O)c4ccc(-n5nnc6cccnc65)cc4F)[C@@H]4CCCNC4)nccc3s2)s1]

Example 425 was prepared according to the procedure described for Example 161 by substituting 4-pyridylboronic acid with (5-cyanothiophen-2-yl)boronic acid, provided the title compound hydrochloride salt as a white solid. LC-MS: [M+H]=581.3. ¹H NMR (400 MHZ, DMSO-d₆): δ 9.06-8.92 (m, 1H), 8.81 (br d, J=4.0 Hz, 2H), 8.68 (br d, J=8.1 Hz, 1H), 8.43 (br d, J=5.5 Hz, 1H), 8.19-7.97 (m, 3H), 7.91 (br d, J=8.6 Hz, 1H), 7.86-7.71 (m, 1H), 7.60 (dd, J=4.6, 8.3 Hz, 1H), 7.56-7.46 (m, 1H), 5.13 (br s, 1H), 3.75 (br d, J=10.5 Hz, 1H), 3.33-3.27 (m, 1H), 3.26-3.12 (m, 1H), 2.72 (br d, J=9.6 Hz, 1H), 2.05 (br d, J=9.0 Hz, 1H), 1.96-1.63 (m, 2H), 1.29-1.04 (m, 1H); ¹⁹F NMR (376 MHz, DMSO-d₆): δ −111.01 (s, 1F).

### Synthesis of 2-fluoro-N-[2-[5-(hydroxymethyl)-2-thienyl] thieno [3, 2-c] pyridin-4-yl]-N-[(3R)-3-piperidyl]-4-(triazolo [4, 5-b] pyridin-3-yl) benzamide (Example 426)

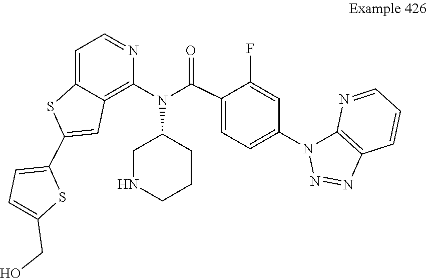 [SMILES: O=C(c1ccc(-n2nnc3cccnc32)cc1F)N(c1nccc2sc(-c3ccc(CO)s3)cc12)[C@@H]1CCCNC1]

Example 426 was prepared according to the procedure described for Example 161 by substituting 4-pyridylboronic acid with [5-(hydroxymethyl)-2-thienyl]boronic acid, provided the title compound hydrochloride salt as a white solid. LC-MS: [M+H]=586.3. ¹H NMR (400 MHz, DMSO-d₆): δ 9.08-8.92 (m, 1H), 8.91-8.74 (m, 2H), 8.68 (br d, J=8.1 Hz, 1H), 8.48-8.29 (m, 1H), 8.26-7.94 (m, 2H), 7.94-7.81 (m, 1H), 7.78-7.67 (m, 1H), 7.64-7.55 (m, 1H), 7.54-7.37 (m, 2H), 7.01 (br d, J=3.5 Hz, 1H), 5.93-5.45 (m, 1H), 5.22-4.83 (m, 1H), 4.80-4.54 (m, 2H), 3.84-3.65 (m, 1H), 3.24-3.14 (m, 1H), 2.91-2.68 (m, 1H), 2.09-1.67 (m, 3H), 1.26-1.03 (m, 1H); ¹⁹F NMR (377 MHz, DMSO-d₆): δ −111.15 (br s, 1F).

### Synthesis of 2-fluoro-N-[2-[2-(hydroxymethyl)thiazol-4-yl]thieno[3,2-c]pyridin-4-yl]-N-[(3R)-3-piperidyl]-4-(triazolo[4,5-b]pyridin-3-yl)benzamide (Example 427)

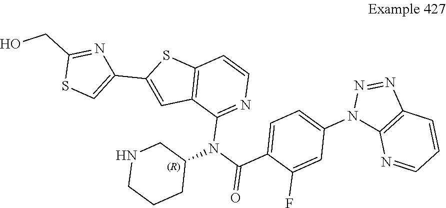

Example 427 was prepared according to the procedure described for Example 161 by substituting 4-pyridylboronic acid with 4-(4,4,5,5-Tetramethyl-1,3,2-dioxaborolan-2-yl)-2-thiazolemethanol, provided the title compound hydrochloride salt as an off white solid. LC-MS: [M+H⁺]=587.3. ¹H NMR (400 MHZ, DMSO-d₆): δ 9.17-8.86 (m, 2H), 8.80 (br d, J=4.3 Hz, 1H), 8.67 (br d, J=8.1 Hz, 1H), 8.51-8.41 (m, 1H), 8.39-8.31 (m, 1H), 8.14 (s, 1H), 8.03-7.95 (m, 2H), 7.88 (br d, J=8.3 Hz, 1H), 7.59 (dd, J=4.6, 8.3 Hz, 1H), 7.55-7.47 (m, 1H), 5.20-4.91 (m, 1H), 4.78 (s, 2H), 3.80-3.68 (m, 1H), 3.20 (br d, J=10.5 Hz, 1H), 2.89-2.64 (m, 2H), 2.13-2.01 (m, 1H), 1.98-1.71 (m, 3H), 1.30-1.09 (m, 1H).

### Synthesis of 2-fluoro-N-[2-(2-hydroxyphenyl)thieno[3,2-c]pyridin-4-yl]-N-[(3R)-3-piperidyl]-4-(triazolo[4,5-b]pyridin-3-yl)benzamide (Example 428)

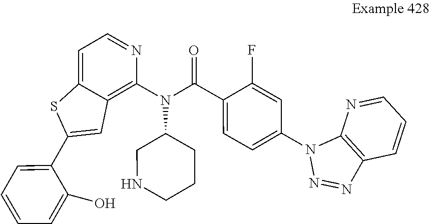 [SMILES: O=C(c1ccc(-n2nnc3cccnc32)cc1F)N(c1nccc2sc(-c3ccccc3O)cc12)[C@@H]1CCCNC1]

Example 428 was prepared according to the procedure described for Example 161 by substituting 4-pyridylboronic acid with (2-hydroxyphenyl)boronic acid, provided the title compound hydrochloride salt as a light yellow solid. LC-MS: [M+H⁺]=566.3. ¹H NMR (400 MHZ, DMSO-d₆): δ 8.80 (br d, J=3.5 Hz, 1H), 8.66 (br d, J=7.8 Hz, 1H), 8.29 (br d, J=1.1 Hz, 1H), 8.03-7.95 (m, 2H), 7.91 (br d, J=5.1 Hz, 1H), 7.84 (br d, J=7.5 Hz, 2H), 7.58 (br dd, J=4.2, 7.7 Hz, 1H), 7.43 (br d, J=4.5 Hz, 1H), 7.30-7.25 (m, 1H), 7.04-6.94 (m, 3H), 4.79-4.56 (m, 1H), 2.93-2.74 (m, 2H), 2.25-2.12 (m, 2H), 2.00-1.90 (m, 1H), 1.80-1.62 (m, 1H), 1.60-1.43 (m, 2H), 1.11-0.97 (m, 1H).

### Synthesis of methyl 4-[4-[[2-fluoro-4-(triazolo[4,5-b]pyridin-3-yl)benzoyl]-[(3R)-3-piperidyl]amino]thieno[3,2-c]pyridin-2-yl]thiazole-2-carboxylate (Example 429)

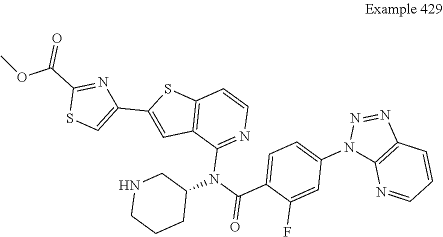 [SMILES: COC(=O)c1nc(-c2cc3c(N(C(=O)c4ccc(-n5nnc6cccnc65)cc4F)[C@@H]4CCCNC4)nccc3s2)cs1]

Example 429 was prepared according to the procedure described for Example 161 by substituting 4-pyridylboronic acid with (2-methoxycarbonylthiazol-4-yl)boronic acid, provided the title compound hydrochloride salt as an off white solid. LC-MS: [M+H⁺]=615.3. ¹H NMR (400 MHz, DMSO-d₆): δ 9.02-8.76 (m, 4H), 8.67 (d, J=7.6 Hz, 1H), 8.43-8.36 (m, 1H), 8.35-8.27 (m, 1H), 8.05 (d, J=5.4 Hz, 1H), 7.98 (br d, J=10.1 Hz, 1H), 7.89 (br d, J=8.8 Hz, 1H), 7.63-7.50 (m, 2H), 5.32-4.87 (m, 1H), 3.95 (s, 3H), 3.81-3.69 (m, 1H), 3.25-3.14 (m, 1H), 2.89-2.71 (m, 1H), 2.15-2.01 (m, 1H), 1.99-1.66 (m, 3H), 1.20-1.10 (m, 1H).

### Synthesis of 2-fluoro-N-[4-[(E)-2-(1-methylpyrazol-4-yl)vinyl]-2-pyridyl]-N-[(3R)-3-piperidyl]-4-(triazolo[4,5-b]pyridin-3-yl)benzamide (Example 430)

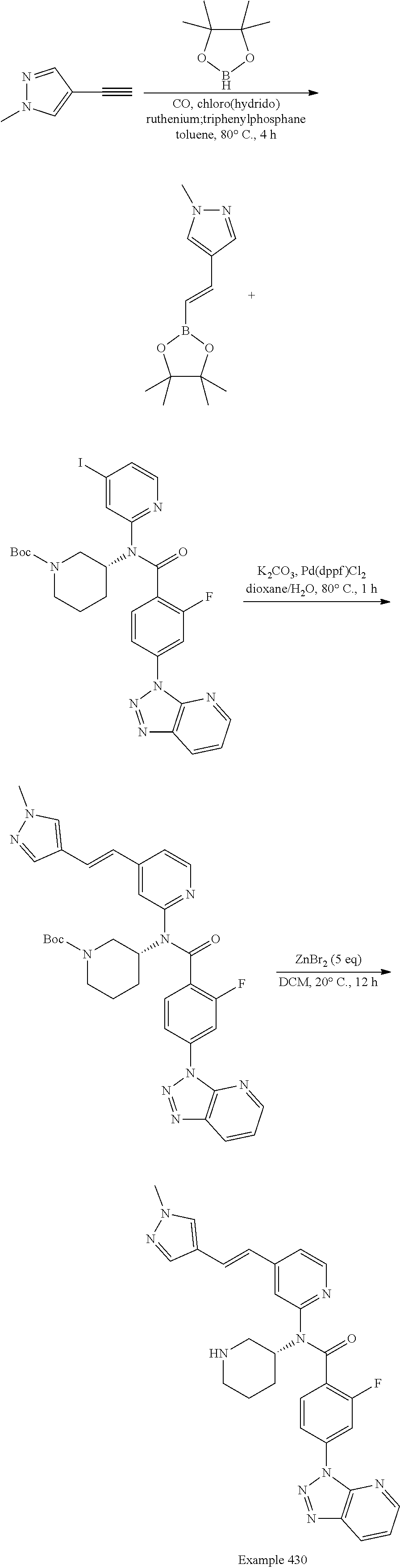

### Step 1:

A mixture of 4-ethynyl-1-methyl-pyrazole (100 mg, 942.29 μmol), 4,4,5,5-tetramethyl-1,3,2-dioxaborolane (132.65 mg, 1.04 mmol), carbon monoxide; chloro(hydrido)-ruthenium; triphenylphosphane (26.92 mg, 28.27 μmol) in toluene (2 mL) was degassed and purged with N₂ for 3 times at 25° C., and then the mixture was stirred at 80° C. for 4 h under N₂ atmosphere. The reaction mixture was filtered and the filtrate was concentrated. The residue was purified by Prep-TLC (SiO₂, Petroleum ether/Ethyl acetate=1/1) to give 1-methyl-4-[(E)-2-(4,4,5,5-tetramethyl-1,3,2-dioxaborolan-2-yl)vinyl]pyrazole (120 mg, 54%) as a white solid.

### Step 2:

A mixture of tert-butyl (3R)-3-[[2-fluoro-4-(triazolo[4,5-b]pyridin-3-yl)benzoyl]-(4-iodo-2-pyridyl)amino]piperidine-1-carboxylate (100 mg, 155.41 μmol), 1-methyl-4-[(E)-2-(4,4,5,5-tetramethyl-1,3,2-dioxaborolan-2-yl)vinyl]pyrazole (47.30 mg, 202.04 μmol), K₂CO₃ (64.44 mg, 466.24 μmol), Pd(dppf)Cl₂ (11.37 mg, 15.54 μmol) in dioxane (1.6 mL) and H₂O (0.4 mL) was degassed with N₂. The mixture was heated to 80° C. and stirred for 1 h under N₂ atmosphere before it was concentrated. The residue was purified by Prep-HPLC (column: Phenomenex luna C18 100*40 mm*5 μm; mobile phase: [H₂O (0.04% HCl)-MeCN]; gradient: 35%-75% B over 8.0 min) to give tert-butyl (3R)-3-[[2-fluoro-4-(triazolo[4,5-b]pyridin-3-yl)benzoyl]-[4-[(E)-2-(1-methylpyrazol-4-yl)vinyl]-2-pyridyl]amino]piperidine-1-carboxylate (30 mg, 30%) as a white solid. LC-MS: [M+H⁺]=624.4.

### Step 3:

To a solution of tert-butyl (3R)-3-[[2-fluoro-4-(triazolo[4,5-b]pyridin-3-yl)benzoyl]-[4-[(E)-2-(1-methylpyrazol-4-yl)vinyl]-2-pyridyl]amino]piperidine-1-carboxylate (28 mg, 44.89 μmol) in CH₂Cl₂ (2 mL) was added ZnBr₂ (50.55 mg, 224.47 μmol) at 25° C. The mixture was stirred at room temperature for 12 h. The reaction mixture was filtered and the filtrate was concentrated to give a residue. The residue was purified by Prep-HPLC (column: Waters Xbridge BEH C18 100*30 mm*10 μm; mobile phase: [H₂O (10 mM NH₄HCO₃)-MeCN]; gradient: 20%-50% B over 8.0 min) to give the title compound (9.7 mg, 41%) as an off-white solid. LC-MS: [M+H⁺]=524.2. ¹H NMR (400 MHZ, CD₃OD): δ 8.83-8.73 (m, 1H), 8.58-8.49 (m, 1H), 8.32-8.13 (m, 2H), 7.85-7.65 (m, 2H), 7.63-7.50 (m, 2H), 7.38-7.13 (m, 3H), 6.89-6.73 (m, 1H), 6.64-6.22 (m, 1H), 4.76-4.54 (m, 1H), 3.87 (s, 3H), 3.51-3.39 (m, 1H), 3.08-2.84 (m, 2H), 2.56-2.41 (m, 1H), 2.24-1.94 (m, 2H), 1.92-1.53 (m, 3H).

### Synthesis of 2-fluoro-N-[(3R)-3-piperidyl]-N-[4-[(E)-2-(3-pyridyl)vinyl]-2-pyridyl]-4-(triazolo[4,5-b]pyridin-3-yl)benzamide (Example 431)

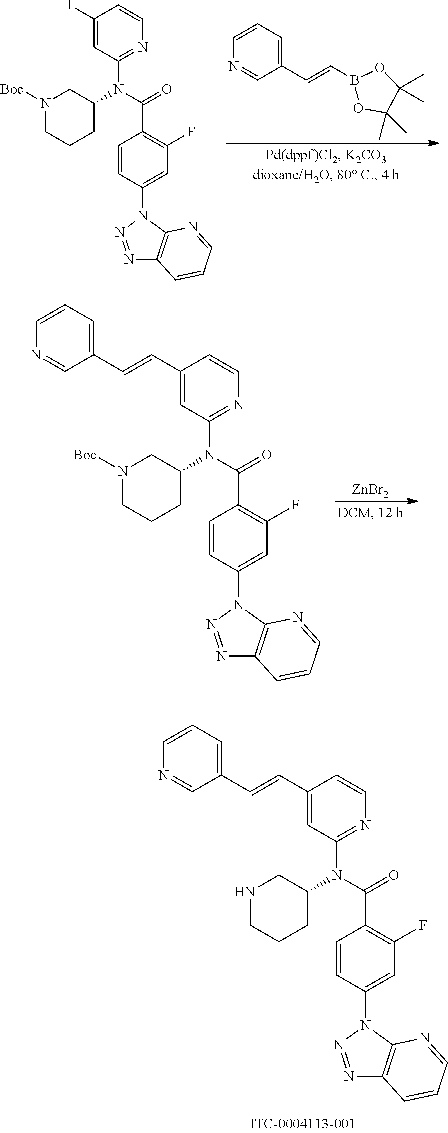

Example 431 was prepared according to the procedure described for Example 430 by substituting 1-methyl-4-[(E)-2-(4,4,5,5-tetramethyl-1,3,2-dioxaborolan-2-yl)vinyl]pyrazole with 3-[(E)-2-(4,4,5,5-tetramethyl-1,3,2-dioxaborolan-2-yl)vinyl]pyridine, provided the title compound hydrochloride salt as a white solid. LC-MS: [M+H⁺]=521.4. ¹H NMR (400 MHz, CD₃OD): δ 8.92-8.67 (m, 2H), 8.58-8.49 (m, 1H), 8.47-8.38 (m, 2H), 8.22-8.05 (m, 2H), 8.00-7.93 (m, 1H), 7.64-7.57 (m, 1H), 7.54 (dd, J=4.6, 8.4 Hz, 1H), 7.50-7.38 (m, 3H), 7.30-7.18 (m, 1H), 7.10-6.96 (m, 1H), 6.55-6.46 (m, 1H), 4.74-4.57 (m, 2H), 3.81 (br s, 1H), 3.69-3.54 (m, 1H), 3.50-3.39 (m, 1H), 3.02-2.87 (m, 1H), 2.53-2.00 (m, 2H), 1.93-1.56 (m, 3H).

### Synthesis of (R)-2-fluoro-N-(8-methylisoquinolin-1-yl)-N-(piperidin-3-yl)-4-(5,6,7,8-tetrahydro-[1,2,4]triazolo[4,3-a]pyridin-3-yl)benzamide (Example 432)

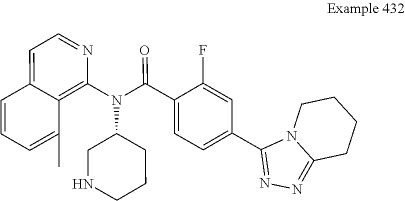 [SMILES: Cc1cccc2ccnc(N(C(=O)c3ccc(-c4nnc5n4CCCC5)cc3F)[C@@H]3CCCNC3)c12]

Example 432 was prepared according to the procedure for Example 127 by substituting 2-bromo-5-methyl-1,3,4-thiadiazole with 3-bromo-5,6,7,8-tetrahydro-[1,2,4]triazolo[4,3-a]pyridine, provided the title compound hydrochloride salt as an off-white solid. LC-MS: (M+1)=485.30. ¹H NMR (CD₃OD, 400 MHZ): δ ppm 8.43-8.40 (m, 1H), 7.74-7.70 (m, 1H), 7.67-7.63 (m, 1H), 7.55-7.51 (m, 2H), 7.33-7.26 (m, 1H), 7.20-7.18 (m, 1H), 7.13-7.09 (m, 1H), 5.10-5.04 (m, 1H), 4.04-3.97 (m, 2H), 3.84-3.79 (m, 1H), 3.47-3.34 (m, 1H), 3.13-3.12 (m, 2H), 3.04-3.00 (m, 1H), 2.89-2.86 (m, 1H), 2.86 (s, 3H), 2.74-2.72 (m, 1H), 2.01-1.92 (m, 6H), 1.59-1.53 (m, 1H).

### Synthesis of ((R)-2-fluoro-N-(piperidin-3-yl)-N-(2-(pyridin-4-yl)thieno[3,2-c]pyridin-4-yl)-4-(pyrimidin-2-ylamino)benzamide (Example 433)

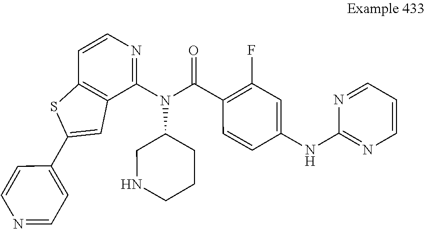 [SMILES: O=C(c1ccc(Nc2ncccn2)cc1F)N(c1nccc2sc(-c3ccncc3)cc12)[C@@H]1CCCNC1]

Example 468 was prepared according to the procedure for Example 119 by substituting tert-butyl (R)-3-((1-methyl-1H-pyrazolo[3,4-c]pyridin-7-yl)amino)piperidine-1-carboxylate with tert-butyl (R)-3-((3-methylthieno[3,2-c]pyridin-4-yl)amino)piperidine-1-carboxylate, provided the title compound hydrochloride salt as an off-white solid. LC-MS: [M+H]=526.2. ¹H NMR (400 MHZ, DMSO-d₆): δ ppm 9.84 (br s, 1H), 9.31-9.13 (m, 1H), 8.98-8.87 (m, 2H), 8.51-8.44 (m, 3H), 8.23 (br s, 2H), 8.12 (br d, J=5.1 Hz, 1H), 7.61-7.45 (m, 1H), 7.09 (br s, 2H), 6.89 (br t, J=4.6 Hz, 1H), 5.12 (br dd, J=3.6, 9.9 Hz, 1H), 3.67 (br s, 1H), 3.22-3.15 (m, 2H), 2.67 (br d, J=1.6 Hz, 1H), 2.08-1.74 (m, 3H), 1.24-1.09 (m, 1H).

### Synthesis of (R)-4-(3H-[1,2,3]triazolo[4,5-b]pyridin-3-yl)-N-(2-([1,2,4]triazolo[1,5-a]pyridin-6-yl)thieno[3,2-c]pyridin-4-yl)-2-fluoro-N-(piperidin-3-yl)benzamide (Example 434)

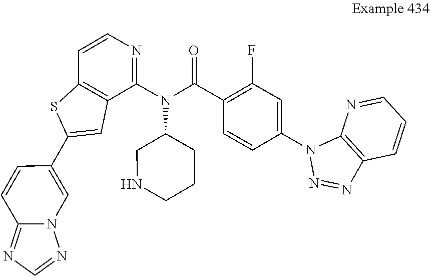 [SMILES: O=C(c1ccc(-n2nnc3cccnc32)cc1F)N(c1nccc2sc(-c3ccc4ncnn4c3)cc12)[C@@H]1CCCNC1]

Example 435 was prepared according to the procedure described for Example 161 by substituting 4-pyridylboronic acid with 6-(4,4,5,5-tetramethyl-1,3,2-dioxaborolan-2-yl)-[1,2,4]triazolo[1,5-a]pyridine, provided the title compound hydrochloride salt as a white solid. LC-MS: [M+H⁺]=591.3. ¹H NMR (400 MHZ, CD₃OD): δ 9.38 (s, 1H), 8.76-8.65 (m, 2H), 8.50 (br d, J=8.4 Hz, 1H), 8.44 (br d, J=5.5 Hz, 1H), 8.28 (br d, J=8.6 Hz, 1H), 8.13-8.04 (m, 2H), 8.01 (br d, J=9.3 Hz, 2H), 7.94 (br d, J=5.4 Hz, 1H), 7.56-7.43 (m, 2H), 5.37-5.22 (m, 1H), 4.01-3.88 (m, 1H), 3.77-3.65 (m, 1H), 3.37 (br d, J=12.4 Hz, 1H), 2.98-2.82 (m, 1H), 2.56-2.22 (m, 1H), 2.20-2.09 (m, 1H), 2.03-1.86 (m, 2H), 1.50-1.34 (m, 1H).

### Synthesis of 2-fluoro-N-[(3R)-3-piperidyl]-N-[2-[1-(2-pyridyl)pyrazol-4-yl]thieno[3,2-c]pyridin-4-yl]-4-(triazolo[4,5-b]pyridin-3-yl)benzamide (Example 435

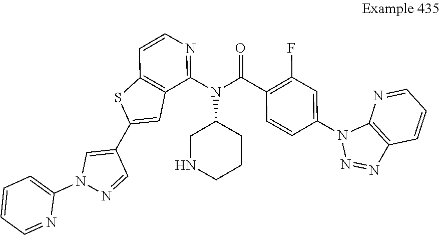 [SMILES: O=C(c1ccc(-n2nnc3cccnc32)cc1F)N(c1nccc2sc(-c3cnn(-c4ccccn4)c3)cc12)[C@@H]1CCCNC1]

Example 435 was prepared according to the procedure described for Example 161 by substituting 4-pyridylboronic acid with 2-[4-(4,4,5,5-tetramethyl-1,3,2-dioxaborolan-2-yl)pyrazol-1-yl]pyridine, provided the title compound hydrochloride salt as a white solid. LC-MS: [M+H⁺]=617.4. ¹H NMR (400 MHZ, DMSO-d₆): δ 9.37-9.26 (m, 1H), 9.15-8.82 (m, 3H), 8.79 (d, J=4.3 Hz, 1H), 8.67 (d, J=8.3 Hz, 1H), 8.55 (br d, J=3.6 Hz, 1H), 8.44-8.40 (m, 1H), 8.35-8.30 (m, 1H), 8.13-7.96 (m, 6H), 7.90 (br d, J=8.5 Hz, 1H), 7.59 (dd, J=4.6, 8.3 Hz, 2H), 7.48-7.41 (m, 1H), 5.19-4.95 (m, 1H), 3.75 (br d, J=10.0 Hz, 1H), 3.39-3.30 (m, 1H), 3.20 (br d, J=9.8 Hz, 1H), 2.72 (br d, J=11.3 Hz, 1H), 2.12 (br d, J=12.4 Hz, 1H), 1.95-1.68 (m, 3H), 1.26-1.07 (m, 1H).

### Synthesis of N-[2-(1-ethylpyrazol-4-yl)thieno[3,2-c]pyridin-4-yl]-2-fluoro-N-[(3R)-3-piperidyl]-4-(triazolo[4,5-b]pyridin-3-yl)benzamide (Example 436)

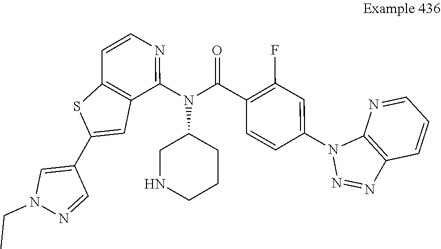 [SMILES: CCn1cc(-c2cc3c(N(C(=O)c4ccc(-n5nnc6cccnc65)cc4F)[C@@H]4CCCNC4)nccc3s2)cn1]

Example 436 was prepared according to the procedure described for Example 161 by substituting 4-pyridylboronic acid with 1-ethyl-4-(4,4,5,5-tetramethyl-1,3,2-dioxaborolan-2-yl)pyrazole, provided the title compound hydrochloride salt as a white solid. LC-MS: [M+H⁺]=568.4. ¹H NMR (400 MHZ, DMSO-d₆): δ 8.80 (br d, J=3.9 Hz, 1H), 8.72-8.65 (m, 1H), 8.73-8.58 (m, 1H), 8.54-8.34 (m, 1H), 8.30-8.06 (m, 1H), 8.05-7.92 (m, 2H), 7.92-7.77 (m, 2H), 7.76-7.55 (m, 2H), 7.52-7.40 (m, 1H), 4.77-4.54 (m, 1H), 4.19 (q, J=7.0 Hz, 2H), 3.29-2.97 (m, 2H), 2.93-2.73 (m, 1H), 2.28-2.08 (m, 2H), 2.03-1.85 (m, 1H), 1.83-1.60 (m, 1H), 1.60-1.48 (m, 1H), 1.43 (t, J=7.3 Hz, 3H), 1.08-0.70 (m, 1H). ¹⁹F NMR (377 MHz, DMSO-d₆): δ −111.08 (br s, 1F).

### Synthesis of (R)-4-(3H-[1,2,3]triazolo[4,5-b]pyridin-3-yl)-2-fluoro-N-(2-(1-isopropyl-1H-pyrazol-4-yl)thieno[3,2-c]pyridin-4-yl)-N-(piperidin-3-yl)benzamide (Example 437)

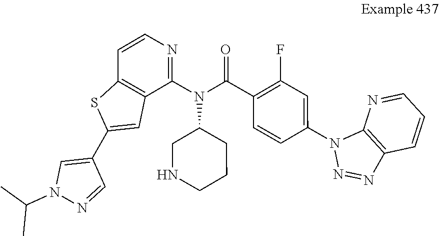 [SMILES: CC(C)n1cc(-c2cc3c(N(C(=O)c4ccc(-n5nnc6cccnc65)cc4F)[C@@H]4CCCNC4)nccc3s2)cn1]

Example 436 was prepared according to the procedure described for Example 161 by substituting 4-pyridylboronic acid 1-isopropyl-4-(4,4,5,5-tetramethyl-1,3,2-dioxaborolan-2-yl)pyrazole, provided the title compound hydrochloride salt as a white solid. LC-MS: [M+H⁺]=582.4.

### Synthesis of 2-fluoro-N-[2-(1-methylpyrazol-3-yl)thieno[3,2-c]pyridin-4-yl]-N-[(3R)-3-piperidyl]-4-(triazolo[4,5-b]pyridin-3-yl)benzamide (Example 438)

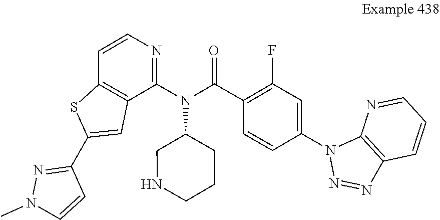 [SMILES: Cn1ccc(-c2cc3c(N(C(=O)c4ccc(-n5nnc6cccnc65)cc4F)[C@@H]4CCCNC4)nccc3s2)n1]

Example 438 was prepared according to the procedure described for Example 161 by substituting 4-pyridylboronic acid with 1-methyl-3-(4,4,5,5-tetramethyl-1,3,2-dioxaborolan-2-yl)pyrazole, provided the title compound hydrochloride salt as off white solid. LC-MS: [M+H⁺]=554.4. ¹H NMR (400 MHZ, DMSO-d₆): δ 9.12-8.85 (m, 2H), 8.80 (br d, J=4.0 Hz, 1H), 8.67 (d, J=8.0 Hz, 1H), 8.36-8.29 (m, 1H), 8.03-7.94 (m, 2H), 7.93-7.83 (m, 3H), 7.60 (dd, J=4.5, 8.3 Hz, 1H), 7.54-7.44 (m, 1H), 7.05-6.97 (m, 1H), 5.19-4.94 (m, 1H), 3.89 (s, 3H), 3.79-3.71 (m, 2H), 3.47-3.40 (m, 1H), 3.37-3.11 (m, 2H), 2.78-2.70 (m, 1H), 2.09-1.99 (m, 1H), 1.87-1.72 (m, 2H), 1.26-1.06 (m, 1H).

### Synthesis of (R)-4-(3H-[1,2,3]triazolo[4,5-b]pyridin-3-yl)-2-fluoro-N-(6-(1-isopropyl-1H-pyrazol-5-yl)isoquinolin-1-yl)-N-(piperidin-3-yl)benzamide (Example 439)

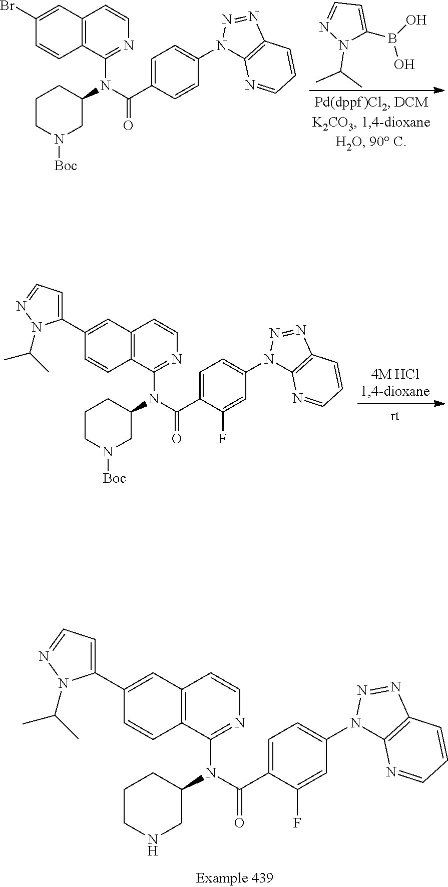

Example 439 was prepared according to the procedure for Example 97 by substituting 1H-pyrazol-4-yl-boronic acid with (1-isopropyl-1H-pyrazol-5-yl)boronic acid, provided the title compound hydrochloride salt as an off-white solid. LC-MS: [M+H⁺]=576.3. ¹H NMR (CD₃OD, 400 MHZ): δ 8.75-8.74 (m, 1H), 8.55-8.49 (m, 2H), 8.30 (d, J=8.8 Hz, 1H), 8.04 (dd, J=1.6 Hz, J=11.2 Hz, 1H), 7.93-7.89 (m, 3H), 7.76-7.74 (m, 1H), 7.59-7.57 (m, 1H), 7.55-7.51 (m, 1H), 7.35-7.31 (m, 1H), 6.42-6.39 (m, 1H), 5.34-5.31 (m, 1H), 4.43-4.41 (m, 1H), 4.20-3.98 (m, 1H), 3.78-3.76 (m, 1H), 2.93-2.89 (m, 1H), 2.10-1.93 (m, 2H), 1.43-1.27 (m, 7H), 0.94-0.89 (m, 2H).

### Synthesis of (R)-4-(3H-[1,2,3]triazolo[4,5-b]pyridin-3-yl)-2-fluoro-N-(6-(2-oxo-1,2-dihydropyridin-4-yl)isoquinolin-1-yl)-N-(piperidin-3-yl)benzamide (Example 440)

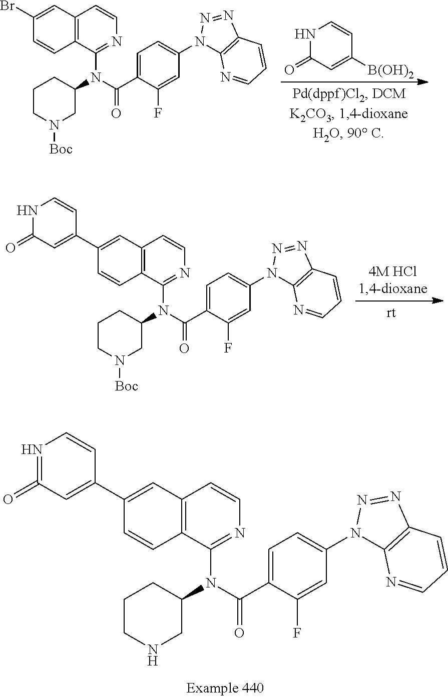

Example 440 was prepared according to the procedure for Example 97 by substituting 1H-pyrazol-4-yl-boronic acid with (2-oxo-1,2-dihydropyridin-4-yl)boronic acid, provided the title compound hydrochloride salt as an off-white solid. LC-MS: [M+H⁺]=561.25. ¹H NMR (CD₃OD, 400 MHZ): δ 8.73-8.70 (m, 1H), 8.57 (d, J=5.6 Hz, 1H), 8.48 (d, J=8.4 Hz, 1H), 8.38-8.30 (m, 2H), 8.17-8.08 (m, 1H), 8.04-8.01 (m, 1H), 7.92-7.91 (m, 3H), 7.52-7.50 (m, 1H), 7.37-7.34 (m, 1H), 7.28-7.27 (m, 1H), 7.21-7.19 (m, 1H), 5.35-5.29 (m, 1H), 4.00-3.98 (m, 1H), 3.76-3.70 (m, 1H), 3.38-3.35 (m, 1H), 2.94-2.84 (m, 1H), 1.96-1.92 (m, 2H), 1.34-1.28 (m, 1H), 0.96-0.87 (m, 1H).

### Synthesis of 2-fluoro-N-(8-methyl-1-isoquinolyl)-N-[(3R, 4S)-4-methyl-3-piperidyl]-4-(1-methyltriazol-4-yl) benzamide (Example 441)

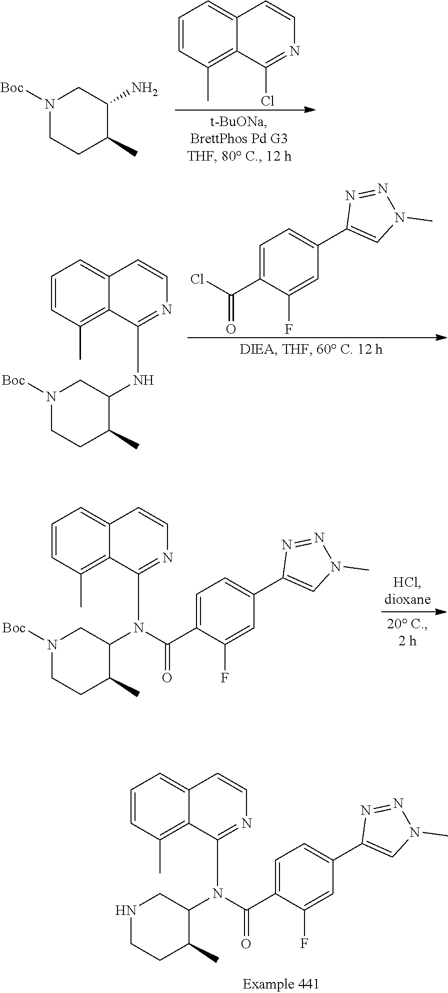

### Step 1:

To a solution of tert-butyl (3R, 4S)-3-amino-4-methyl-piperidine-1-carboxylate (2 g, 9.32 mmol) and 1-chloro-8-methyl-isoquinoline (1.65 g, 9.32 mmol) in tetrahydrofuran (20 mL) was added t-BuONa (2.69 g, 27.97 mmol) and BrettPhos Pd G3 (253.54 mg, 279.70 μmo) at 20° C. under N₂. The mixture was heated to 80° C. and stirred for 12 h under N₂. The reaction mixture was filtered and the filtrate was concentrated. The residue was purified by column chromatography (SiO₂, Petroleum ether/Ethyl acetate=5/1 to 4/1) to give tert-butyl (3R, 4S)-4-methyl-3-[(8-methyl-1-isoquinolyl) amino] piperidine-1-carboxylate (2.9 g, 87%) as a white solid.

### Step 2:

### Step 2:

To a solution of tert-butyl (3R, 4S)-4-methyl-3-[(8-methyl-1-isoquinolyl)amino] piperidine-1-carboxylate (2.9 g, 8.16 mmol) in tetrahydrofuran (20 mL) was added DIEA (8.43 g, 65.21 mmol) and 2-fluoro-4-(1-methyltriazol-4-yl) benzoyl chloride (4.76 g, 20.38 mmol) at 0° C. under N₂. The mixture was heated to 60° C. and stirred for 12 h under N₂ before it was concentrated. The residue was purified by column chromatography (SiO₂, Petroleum ether/Ethyl acetate=5/2 to 1/1) to give tert-butyl (3R, 4S)-3-[[2-fluoro-4-(1-methyltriazol-4-yl) benzoyl]-(8-methyl-1-isoquinolyl) amino]-4-methyl-piperidine-1-carboxylate (4.1 g, 89%) as a white solid.

### Step 3:

To a solution of tert-butyl (3R, 4S)-3-[[2-fluoro-4-(1-methyltriazol-4-yl) benzoyl]-(8-methyl-1-isoquinolyl) amino]-4-methyl-piperidine-1-carboxylate (3.6 g, 6.45 mmol) in 4M HCl in dioxane (36 mL) was stirred at 20° C. for 2 h. The reaction mixture was concentrated. The residue was triturated with EtOAc:MeOH=10:1 (20 mL). The precipitate was collected and dried to give the title compound hydrochloride salt (2.1 g, 71%) as white solid. LC-MS: [M+H⁺]=459.4. ¹H NMR (400 MHZ, CD₃OD): δ 8.50-8.40 (m, 1H), 8.27-8.20 (m, 1H), 7.76-7.66 (m, 1H), 7.59 (t, J=7.5 Hz, 1H), 7.49-7.34 (m, 2H), 7.26-7.10 (m, 2H), 7.02 (t, J=7.6 Hz, 1H), 5.29-5.17 (m, 1H), 4.90-4.83 (m, 1H), 4.09 (s, 3H), 3.66-3.49 (m, 1H), 3.44 (br d, J=11.8 Hz, 1H), 3.26-2.97 (m, 2H), 2.89-2.79 (m, 3H), 2.19-1.95 (m, 1H), 1.88-1.66 (m, 1H), 1.35 (d, J=6.5 Hz, 2H), 1.00 (d, J=6.5 Hz, 1H).

### Synthesis of N-[2-[(E)-3-amino-3-oxo-prop-1-enyl]thieno[3,2-c]pyridin-4-yl]-2-fluoro-N-[(3R)-3-piperidyl]-4-(triazolo[4,5-b]pyridin-3-yl)benzamide (Example 442)

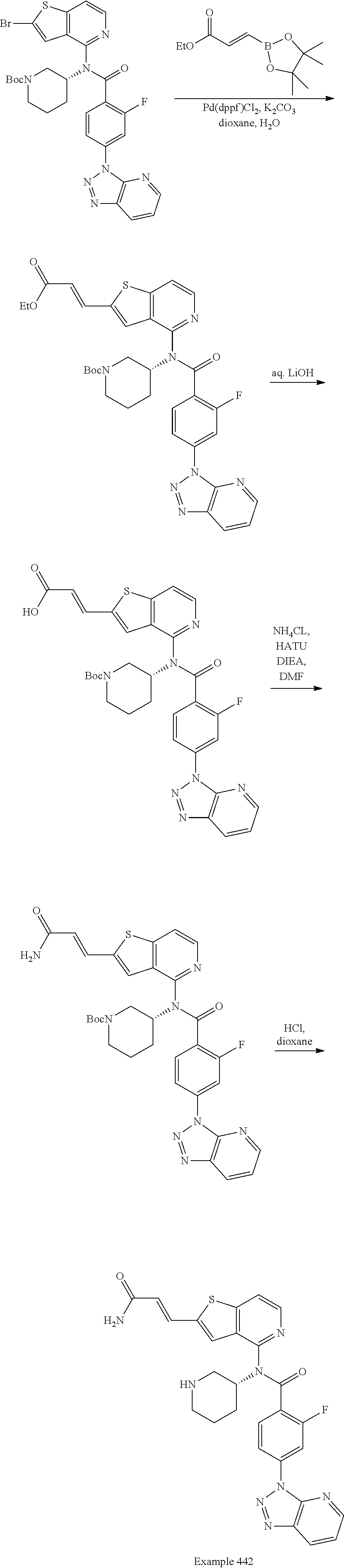 [SMILES: CC(C)(C)OC(=O)N1CCC[C@@H](N(C(=O)c2ccc(-n3nnc4cccnc43)cc2F)c2nccc3sc(Br)cc23)C1 ; CCOC(=O)/C=C/B1OC(C)(C)C(C)(C)O1 ; CCOC(=O)/C=C/c1cc2c(N(C(=O)c3ccc(-n4nnc5cccnc54)cc3F)[C@@H]3CCCN(C(=O)OC(C)(C)C)C3)nccc2s1 ; CC(C)(C)OC(=O)N1CCC[C@@H](N(C(=O)c2ccc(-n3nnc4cccnc43)cc2F)c2nccc3sc(/C=C/C(=O)O)cc23)C1 ; CC(C)(C)OC(=O)N1CCC[C@@H](N(C(=O)c2ccc(-n3nnc4cccnc43)cc2F)c2nccc3sc(/C=C/C(N)=O)cc23)C1 ; NC(=O)/C=C/c1cc2c(N(C(=O)c3ccc(-n4nnc5cccnc54)cc3F)[C@@H]3CCCNC3)nccc2s1]

### Step 1:

A mixture of ethyl (E)-3-(4,4,5,5-tetramethyl-1,3,2-dioxaborolan-2-yl)prop-2-enoate (519.69 mg, 2.30 mmol), tert-butyl (3R)-3-[(2-bromothieno[3,2-c]pyridin-4-yl)-[2-fluoro-4-(triazolo[4,5-b]pyridin-3-yl)benzoyl]amino]piperidine-1-carboxylate (500 mg, 766.24 μmol), K₂CO₃ (317.71 mg, 2.30 mmol), Pd(dppf)Cl₂ (56.07 mg, 76.62 μmol) in dioxane (5 mL) and H₂O (1 mL) at 20° C. was purged with N₂, and then the mixture was stirred at 80° C. for 2 h under N₂ atmosphere. The reaction mixture was filtered and the filtrate was concentrated. The residue was purified by Prep-HPLC (column: Waters Xbridge Prep OBD C18 150*40 mm*10 μm; mobile phase: [H₂O (10 mM NH4HCO3)-MeCN]; gradient: 65%-95% B over 8.0 min) to give tert-butyl (3R)-3-[[2-[(E)-3-ethoxy-3-oxo-prop-1-enyl]thieno[3,2-c]pyridin-4-yl]-[2-fluoro-4-(triazolo[4,5-b]pyridin-3-yl)benzoyl]amino]piperidine-1-carboxylate (340 mg, 66%) as a yellow solid.

### Step 2:

To a solution of tert-butyl (3R)-3-[[2-[(E)-3-ethoxy-3-oxo-prop-1-enyl]thieno[3,2-c]pyridin-4-yl]-[2-fluoro-4-(triazolo[4,5-b]pyridin-3-yl)benzoyl]amino]piperidine-1-carboxylate (340 mg, 506.15 μmol) in MeOH (4 mL) and H₂O (4 mL) was added LiOH·H₂O (106.20 mg, 2.53 mmol). The mixture was stirred at 20° C. for 2 h before the organic volatile was removed. The aq. phase was adjusted to pH 2 with 1N aq. HCl. The precipitate was collected and dried by lyophilization to give (E)-3-[4-[[(3R)-1-tert-butoxycarbonyl-3-piperidyl]-[2-fluoro-4-(triazolo[4,5-b]pyridin-3-yl)benzoyl]amino]thieno[3,2-c]pyridin-2-yl]prop-2-enoic acid (300 mg, 92%) as a yellow solid.

### Step 3:

A mixture of (E)-3-[4-[[(3R)-1-tert-butoxycarbonyl-3-piperidyl]-[2-fluoro-4-(triazolo[4,5-b]pyridin-3-yl)benzoyl]amino]thieno[3,2-c]pyridin-2-yl]prop-2-enoic acid (100 mg, 155.36 μmol), NH₄Cl (24.93 mg, 466.07 μmol), DIEA (60.23 mg, 466.07 μmol) in DMF (2 mL) was stirred at 25° C. for 1 h. Then HATU (70.88 mg, 186.43 μmol) was added, and the reaction was stirred at 25° C. for 11 h. before it was concentrated. The residue was purified by Prep-HPLC (column: Waters Xbridge Prep OBD C18 150*40 mm*10 μm; mobile phase: [H₂O (10 mM NH₄HCO₃)-MeCN]; gradient: 40%-70% B over 8.0 min) to give tert-butyl (3R)-3-[[2-[(E)-3-amino-3-oxo-prop-1-enyl]thieno[3,2-c]pyridin-4-yl]-[2-fluoro-4-(triazolo[4,5-b]pyridin-3-yl)benzoyl]amino]piperidine-1-carboxylate (50 mg, 50%) as a yellow solid.

### Step 4:

To a solution of tert-butyl (3R)-3-[[2-[(E)-3-amino-3-oxo-prop-1-enyl]thieno[3,2-c]pyridin-4-yl]-[2-fluoro-4-(triazolo[4,5-b]pyridin-3-yl)benzoyl]amino]piperidine-1-carboxylate (50 mg, 77.80 μmol) in DCM (1 mL) was added ZnBr₂ (175.20 mg, 777.97 μmol). The mixture was stirred at 25° C. for 12 h before it was concentrated. The residue was purified by prep-HPLC (column: Waters Xbridge Prep OBD C18 150*40 mm*10 μm; mobile phase: [H₂O (10 mM NH₄HCO₃)-MeCN]; gradient: 10%-50% B over 8.0 min) to afford N-[2-[(E)-3-amino-3-oxo-prop-1-enyl]thieno[3,2-c]pyridin-4-yl]-2-fluoro-N-[(3R)-3-piperidyl]-4-(triazolo[4,5-b]pyridin-3-yl)benzamide (29 mg, 68%) as a yellow solid. LC-MS: [M+H⁺]=543.4. ¹H NMR (400 MHz, CD₃OD): δ 8.77 (br s, 1H), 8.51 (br d, J=8.0 Hz, 1H), 8.36 (br s, 1H), 8.08 (br d, J=10.8 Hz, 1H), 8.03-7.95 (m, 1H), 7.86-7.76 (m, 2H), 7.74 (s, 1H), 7.58-7.50 (m, 1H), 7.49-7.36 (m, 1H), 6.56 (br d, J=15.8 Hz, 1H), 4.76-4.52 (m, 1H), 3.53 (br dd, J=2.3, 3.6 Hz, 1H), 3.20-3.06 (m, 1H), 3.00-2.88 (m, 1H), 2.57-2.28 (m, 2H), 2.07-1.90 (m, 1H), 1.88-1.59 (m, 2H), 1.37-1.18 (m, 1H).

### Synthesis of 2-fluoro-N-[2-(2-methylthiazol-5-yl)thieno[3,2-c]pyridin-4-yl]-N-[(3R)-3-piperidyl]-4-(triazolo[4,5-b]pyridin-3-yl)benzamide (Example 443)

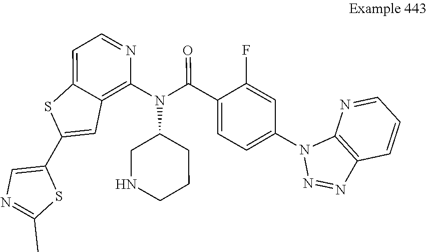 [SMILES: Cc1ncc(-c2cc3c(N(C(=O)c4ccc(-n5nnc6cccnc65)cc4F)[C@@H]4CCCNC4)nccc3s2)s1]

Example 443 was synthesized following the procedure for example 161 by substituting 4-pyridylboronic acid with 2-methyl-5-(4,4,5,5-tetramethyl-1,3,2-dioxaborolan-2-yl) thiazole to afford the title compound hydrochloride salt as an off-white solid. LC-MS: [M+H⁺]=571.3. ¹H NMR (400 MHZ, CD₃OD): δ 8.75 (br s, 1H), 8.58-8.34 (m, 2H), 8.22-7.95 (m, 2H), 7.89 (br s, 1H), 7.78-7.31 (m, 4H), 5.26 (br s, 1H), 4.02-3.79 (m, 1H), 3.77-3.63 (m, 1H), 2.85 (br s, 4H), 2.17-1.79 (m, 3H), 1.52-1.31 (m, 1H).

### Synthesis of N-(6-chloro-8-methyl-1-isoquinolyl)-2-fluoro-4-(1-methyltriazol-4-yl)-N-[(3R)-3-piperidyl] benzamide (Example 444)

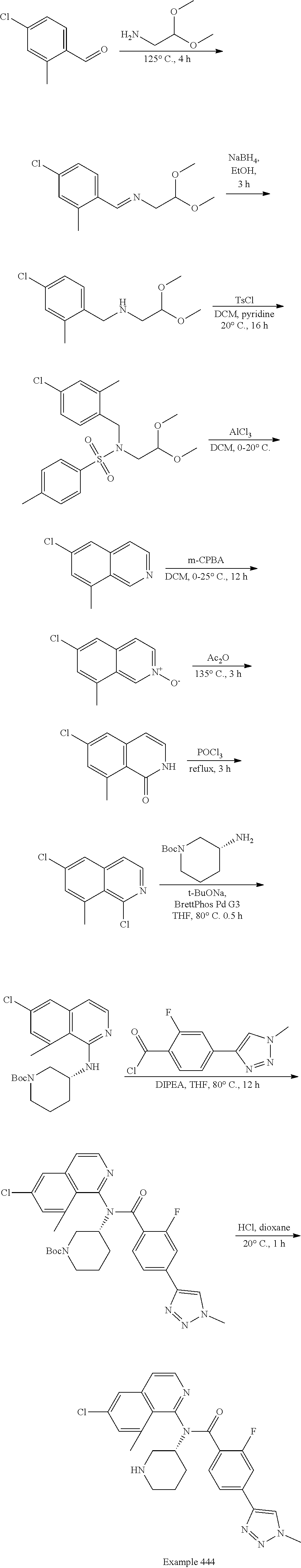

### Step 1:

A mixture of 4-chloro-2-methyl-benzaldehyde (15 g, 97 mmol) and 2,2-dimethoxyethanamine (11.22 g, 106.73 mmol) in toluene (200 mL) was purged with N₂ for thrice at 20° C., and then the mixture was heated to 125° C. and stirred for 4 h under N₂ atmosphere. The reaction mixture was cooled to room temperature and concentrated. The residue was purified by column chromatography (SiO₂, Petroleum ether/Ethyl acetate=50/1 to 48/1) to give (E)-1-(4-chloro-2-methylphenyl)-N-(2,2-dimethoxyethyl) methanimine (20 g, 85%) as a yellowish oil. LC-MS: [M+H⁺]=242.4.

### Step 2:

A mixture of (E)-1-(4-chloro-2-methylphenyl)-N-(2,2-dimethoxyethyl) methanimine (20 g, 82.7 mmol) in EtOH (200 mL) at 0° C. was added NaBH₄ (5.39 g, 142.60 mmol) portion-wise. The mixture was warmed to 20° C. and stirred for 3 h. The reaction mixture was quenched with ice water (100 mL). The mixture was stirred at 20° C. for 1 h. Then the mixture was extracted with ethyl acetate (3×100 mL). The combined organic layer was washed with brine (300 mL), dried over Na₂SO₄, filtered and concentrated. The residue was purified by flash silica gel chromatography (eluting 0-12% EtOAc in petroleum ether) to give N-[(4-chloro-2-methyl-phenyl) methyl]-2, 2-dimethoxy-ethanamine (19.00 g, 94%) as a colorless oil. LC-MS: [M+H⁺]=244.4.

### Step 3:

To a solution of N-[(4-chloro-2-methyl-phenyl)methyl]-2,2-dimethoxy-ethanamine (19 g, 77.9 mmol) and pyridine (32.45 g, 410.29 mmol) in DCM (200 mL) at 20° C. was added 4-methylbenzenesulfonyl chloride (22.20 g, 116.9 mmol). The mixture was stirred at 20° C. for 16 h before it was partitioned between water (300 mL) ethyl acetate (3×200 mL). The combined organic layer was washed with brine (200 mL), dried over Na₂SO₄, filtered and concentrated. The residue was purified by column chromatography (SiO₂, Petroleum ether/Ethyl acetate=15/1 to 12/1) to give N-[(4-chloro-2-methyl-phenyl) methyl]-N-(2, 2-dimethoxyethyl)-4-methyl-benzenesulfonamide (24.40 g, 79%) as a colorless oil.

### Step 4:

A mixture of AlCl₃ (40.90 g, 306.6 mmol) in DCM (300 mL) under N₂ atmosphere at 0° C. was stirred for 0.5 h and followed by addition of N-[(4-chloro-2-methyl-phenyl) methyl]-N-(2, 2-dimethoxyethyl)-4-methyl-benzenesulfonamide (24.40 g, 61.30 mmol). The mixture was stirred at 20° C. for 3 h under N₂ atmosphere and then poured into ice water (1000 mL). The mixture was extracted with ethyl acetate (3×500 mL). The combined organic layer was washed with saturated aqueous Na₂CO₃ solution (2×500 mL), dried over Na₂SO₄, filtered and concentrated. The residue was purified by column chromatography (SiO₂, Petroleum ether/Ethyl acetate=12/1 to 10/1) to give 6-chloro-8-methyl-isoquinoline (9.5 g, 87%) as a colorless oil. LC-MS: [M+H⁺]=178.5.

### Step 5:

To a mixture of 6-chloro-8-methyl-isoquinoline (9.50 g, 53.4 mmol) in DCM (80 mL) was added m-CPBA (13.80 g, 80.1 mmol) at 0° C., and then the mixture was stirred at 25° C. for 12 h. The reaction mixture was quenched by addition of a 20% aq. Na₂SO₃ solution (100 mL) at 0° C. The reaction mixture was diluted with water (100 mL) and extracted with EtOAc (3×100 mL). The combined organic layer was washed with saturated aq. NaHCO₃ solution (2×150 mL), dried over Na₂SO₄, filtered and concentrated to give crude 6-chloro-8-methylisoquinoline 2-oxide (10.10 g) as an oil. The crude product was used for the next step without purification.

### Step 6:

A mixture of crude 6-chloro-8-methylisoquinoline 2-oxide (10.10 g) in Ac₂O (137.08 g, 1.34 mol) at 25° C. was heated to 135° C. and stirred for 3 h under N₂ atmosphere. The reaction was cooled and then concentrated. The residue was purified by flash column chromatography (SiO₂, Petroleum ether/Ethyl acetate=15/1 to 13/1) to give 6-chloro-8-methyl-2H-isoquinolin-1-one (5.20 g, 51%) as a white solid.

### Step 7:

A mixture of 6-chloro-8-methyl-2H-isoquinolin-1-one (5.20 g, 26.8 mmol) in POCl₃ (3.29 g, 21.46 mmol) at 20° C. was purged with N₂, and then the mixture was heated at reflux for 3 h under N₂ atmosphere. The reaction mixture was poured into water (100 mL) at 20° C., and then adjusted pH to 10 with 10M aq. NaOH solution. The mixture was extracted with ethyl acetate (3×60 mL). The combined organic layer was washed with brine (2×90 mL), dried over Na₂SO₄, filtered and concentrated under reduced pressure. The residue was purified by flash column chromatography (SiO₂, Petroleum ether/Ethyl acetate=50/1 to 30/1) to give 1, 6-dichloro-8-methyl-isoquinoline (3.10 g, 54%) as a yellow solid. LC-MS: [M+H⁺]=212.4. ¹H NMR (400 MHZ, DMSO-d₆): δ 8.27 (d, J=5.5 Hz, 1H), 8.06 (s, 1H), 7.82 (d, J=5.5 Hz, 1H), 7.65 (s, 1H), 2.97 (s, 3H).

### Step 8:

A mixture of 1,6-dichloro-8-methyl-isoquinoline (20.00 g, 94.31 mmol), tert-butyl (3R)-3-aminopiperidine-1-carboxylate (11.33 g, 56.58 mmol), [2-(2-aminophenyl)phenyl]-methylsulfonyloxy-palladium; dicyclohexyl-[3,6-dimethoxy-2-(2,4,6-triisopropylphenyl) phenyl] phosphane (2.56 g, 2.82 mmol), sodium; 2-methylpropan-2-olate (27.18 g, 282.93 mmol) in THF (300 mL) at 20° C. was degassed with N₂, and then the mixture was heated to 80° C. and stirred for 0.5 h under N₂ atmosphere. The reaction was cooled and concentrated. The residue was purified by flash column chromatography (SiO₂, Petroleum ether/Ethyl acetate=15/1 to 13/1) to give tert-butyl (3R)-3-[(6-chloro-1-isoquinolyl)amino]piperidine-1-carboxylate (15.00 g, 36%) as an oil. Optical rotation=−15.88° (MeOH, c=0.98 g/100 mL at 20° C., 589 nm). LC-MS: [M+H⁺]=376.3.

### Step 9:

A mixture of 2-fluoro-4-(1-methyltriazol-4-yl)benzoic acid (9.00 g, 40.7 mmol) in SOCl₂ (48.4 g, 406.9 mmol) was heated to 80° C. and stirred for 1 h under N₂ atmosphere. The reaction mixture was concentrated under reduced pressure to give crude 2-fluoro-4-(1-methyltriazol-4-yl)benzoyl chloride (9.59 g) as a white solid.

### Step 10:

To a solution of tert-butyl (3R)-3-[(6-chloro-8-methyl-1-isoquinolyl)amino]piperidine-1-carboxylate (5 g, 13.3 mmol), DIPEA (8.6 g, 66.5 mmol) in THF (100 mL) at 20° C. under N₂ atmosphere was added crude 2-fluoro-4-(1-methyltriazol-4-yl)benzoyl chloride (9.56 g, 39.9 mmol) in portions. The mixture was heated to 60° C. and stirred for 12 h. The reaction was cooled to 0° C., diluted with H₂O (100 mL), and then extracted with EtOAc (3×150 mL). The combined organic layer was washed with brine (150 mL), dried over Na₂SO₄, filtered and concentrated. The residue was purified by flash column chromatography (SiO₂, Petroleum ether/EtOAc=1:1) to give tert-butyl (3R)-3-[(6-chloro-8-methyl-1-isoquinolyl)amino]piperidine-1-carboxylate (3.80 g, 49%) as a yellow solid. LC-MS: [M+H⁺]=579.3.

### Step 11:

To a solution of tert-butyl (3R)-3-[(6-chloro-8-methyl-1-isoquinolyl)-[2-fluoro-4-(1-methyltriazol-4-yl)benzoyl]amino]piperidine-1-carboxylate (3.00 g, 5.18 mmol) in 1,4-dioxane (30 mL) was added a solution of 4M HCl in 1,4-dioxane (14.25 mL). The mixture was stirred at 20° C. for 1 h and then it was concentrated under reduced pressure. The residue was purified by reverse-phase Prep-HPLC (column: Phenomenex luna C18 100*40 mm*5 μm; mobile phase: [H₂O (0.04% HCl)-MeCN]; gradient: 10%-45% B over 8.0 min) and the pure fraction was lyophilized to give the title compound hydrochloride salt (1.50 g, 60%) as a white solid. LC-MS: [M+H⁺]=479.1. ¹H NMR (400 MHZ, CD₃OD): δ (ppm) 8.49-8.41 (m, 1H), 8.18 (s, 1H), 7.73-7.63 (m, 2H), 7.48-7.42 (m, 1H), 7.28 (dd, J=1.3, 11.3 Hz, 1H), 7.17 (dd, J=1.4, 8.1 Hz, 1H), 6.92-6.82 (m, 1H), 5.14-5.01 (m, 1H), 4.07 (s, 3H), 4.02-3.94 (m, 1H), 3.88-3.77 (m, 1H), 3.41 (br d, J=12.3 Hz, 1H), 3.09-2.95 (m, 1H), 2.89-2.80 (m, 3H), 2.79-2.64 (m, 1H), 2.07-1.87 (m, 3H), 1.70-1.51 (m, 1H).

### Synthesis of (R)-2-fluoro-4-(1-methyl-1H-1,2,3-triazol-4-yl)-N-(6-(1-methyl-1H-pyrazol-4-yl)isoquinolin-1-yl)-N-(piperidin-3-yl)benzamide (Example 445)

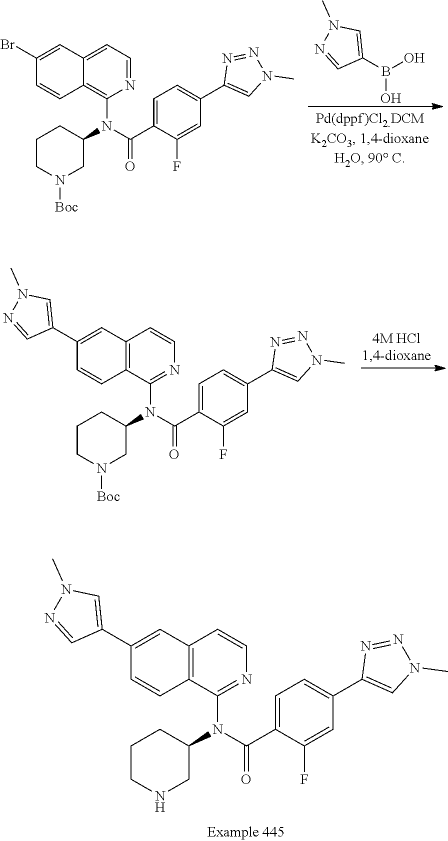

Example 445 was prepared according to the procedure for Example 370 by substituting 1H-pyrazol-4-yl-boronic acid with pyridin-3-ylboronic acid, provided the title compound hydrochloride salt as an off-white solid. LC-MS: [M+H⁺]=511.45. ¹H NMR (CD₃OD, 400 MHZ): δ 8.39-8.36 (m, 1H), 8.24-8.21 (m, 1H), 8.14-8.10 (m, 2H), 8.06-7.99 (m, 2H), 7.95-7.93 (m, 1H), 7.77-7.72 (m, 1H), 7.28-7.26 (m, 1H), 7.21-7.19 (m, 1H), 7.13-7.09 (m, 1H), 5.27-5.21 (m, 1H), 4.04 (s, 3H), 3.99 (s, 3H), 3.96-3.92 (m, 1H), 3.70-3.64 (m, 1H), 2.89-2.82 (m, 1H), 2.04-1.98 (m, 1H), 1.90-1.88 (m, 2H), 1.39-1.28 (m, 2H).

### Synthesis of 2-fluoro-N-[(3R)-3-piperidyl]-N-[2-(3-sulfamoylphenyl)thieno[3,2-c]pyridin-4-yl]-4-(triazolo[4,5-b]pyridin-3-yl)benzamide (Example 446)

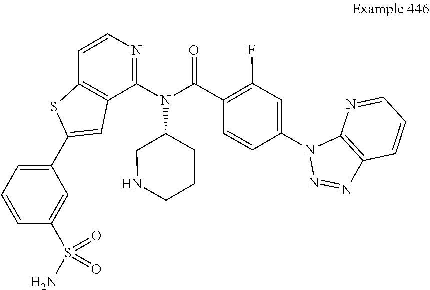 [SMILES: NS(=O)(=O)c1cccc(-c2cc3c(N(C(=O)c4ccc(-n5nnc6cccnc65)cc4F)[C@@H]4CCCNC4)nccc3s2)c1]

Example 446 was synthesized following the procedure for example 161 by substituting 4-pyridylboronic acid with (3-sulfamoylphenyl) boronic acid, provided the title compound as an off-white solid. LC-MS: [M+H⁺]=629.3. ¹H NMR (400 MHZ, DMSO-d₆) δ 9.27 (br d, J=9.0 Hz, 1H), 9.18-8.93 (m, 1H), 8.79 (d, J=3.8 Hz, 1H), 8.67 (d, J=8.1 Hz, 1H), 8.49-8.34 (m, 1H), 8.33-8.05 (m, 3H), 8.03-7.84 (m, 3H), 7.82-7.70 (m, 1H), 7.66-7.45 (m, 3H), 5.24-4.94 (m, 1H), 3.76 (br d, J=11.6 Hz, 1H), 3.38-3.11 (m, 2H), 2.84-2.64 (m, 2H), 2.30-2.06 (m, 1H), 1.98-1.71 (m, 2H), 1.27-1.07 (m, 1H).

### Synthesis of N-[2-[3-(2-amino-2-oxo-ethyl)phenyl]thieno[3,2-c]pyridin-4-yl]-2-fluoro-N-[(3R)-3-piperidyl]-4-(triazolo[4,5-b] pyridin-3-yl)benzamide (Example 447)

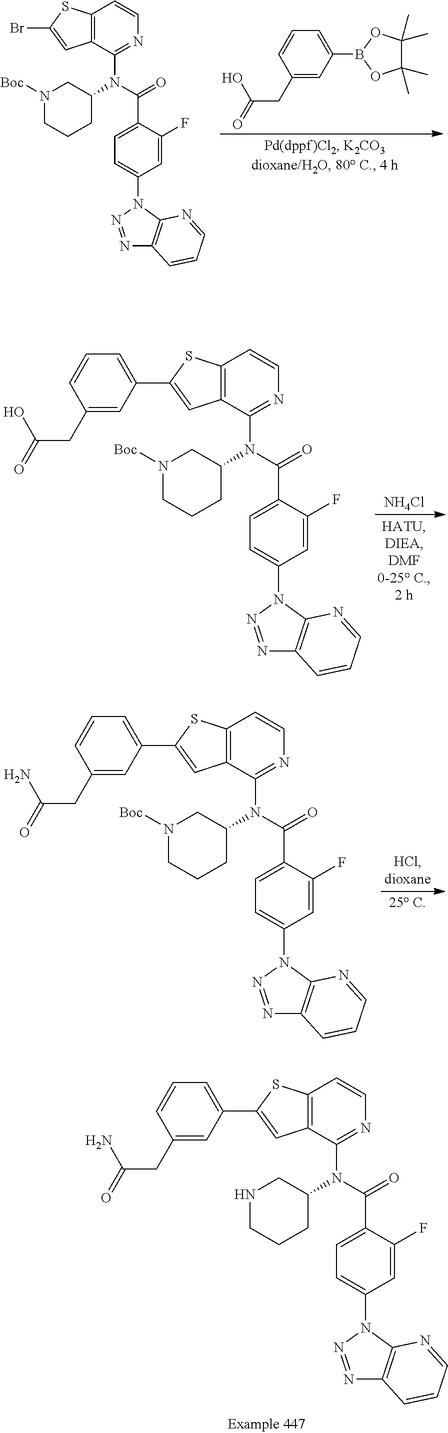

To a solution of tert-butyl (3R)-3-[(2-bromothieno[3,2-c]pyridin-4-yl)-[2-fluoro-4-(triazolo[4,5-b]pyridin-3-yl)benzoyl]amino]piperidine-1-carboxylate (150 mg, 229.87 μmol) in dioxane (2 mL) and H₂O (0.4 mL) was added 2-[3-(4,4,5,5-tetramethyl-1,3,2-dioxaborolan-2-yl) phenyl]acetic acid (120.50 mg, 459.74 μmol), K₂CO₃ (95.31 mg, 689.62 μmol), Pd(dppf)Cl₂ (16.82 mg, 22.99 μmol) under N₂ atmosphere. The mixture was stirred at 80° C. for 4 h before it was cooled, filtered. The filtrate was concentrated. The residue was purified by Prep-HPLC (column: Phenomenex luna C18 100*40 mm*5 μm; mobile phase: [H₂O (0.04% HCl)-MeCN]; gradient: 50%-80% B over 8.0 min) to give 2-[3-[4-[[(3R)-1-tert-butoxycarbonyl-3-piperidyl]-[2-fluoro-4-(triazolo[4,5-b] pyridine-3-yl)benzoyl]amino]thieno[3,2-c]pyridin-2-yl]phenyl]acetic acid (80 mg, 49%) as a yellow solid.

### Step 2:

A solution of 2-[3-[4-[[(3R)-1-tert-butoxycarbonyl-3-piperidyl]-[2-fluoro-4-(triazolo [4,5-b]pyridin-3-yl)benzoyl]amino]thieno[3,2-c]pyridin-2-yl]phenyl]acetic acid (80 mg, 113.03 μmol), NH₄Cl (18.14 mg, 339.09 μmol), and DIEA (43.82 mg, 339.09 μmol) in DMF (2 mL) was stirred at 25° C. for 1 h. Then HATU (51.57 mg, 135.64 μmol) was added and the reaction was stirred at 25° C. for 11 h. The reaction was poured into ice-water (5 mL) and stirred for 20 min. The precipitate was collected and to give the crude product which was used for next step directly without purification.

### Step 3:

The title compound hydrochloride salt was obtained as an off-white solid by deprotection Method A used in Example 97. LC-MS: [M+H⁺]=607.3. ¹H NMR (400 MHZ, CD₃OD): δ 8.74 (br d, J=4.1 Hz, 1H), 8.50 (br d, J=8.3 Hz, 1H), 8.37 (d, J=5.4 Hz, 1H), 8.10 (br d, J=11.3 Hz, 1H), 7.99 (br d, J=8.6 Hz, 1H), 7.87 (d, J=5.6 Hz, 1H), 7.80-7.70 (m, 3H), 7.52 (dd, J=4.4, 8.3 Hz, 1H), 7.49-7.37 (m, 3H), 5.34-5.18 (m, 1H), 4.82 (s, 1H), 3.97-3.82 (m, 1H), 3.73-3.63 (m, 1H), 3.37 (br s, 1H), 2.95-2.82 (m, 1H), 2.51-2.18 (m, 1H), 2.17-2.06 (m, 1H), 2.02-1.85 (m, 2H), 1.49-1.32 (m, 1H).

### Synthesis of N-[2-(4-carbamoylphenyl)thieno[3,2-c]pyridin-4-yl]-2-fluoro-N-[(3R)-3-piperidyl]-4-(triazolo[4,5-b]pyridin-3-yl)benzamide (Example 448)

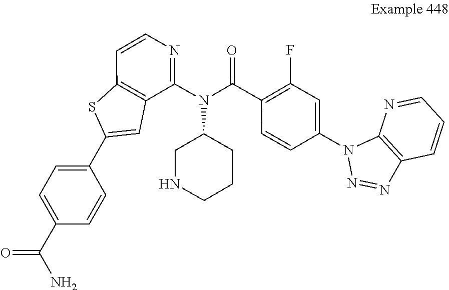 [SMILES: NC(=O)c1ccc(-c2cc3c(N(C(=O)c4ccc(-n5nnc6cccnc65)cc4F)[C@@H]4CCCNC4)nccc3s2)cc1]

Example 448 was prepared according to the procedure described for Example 161 by substituting 4-pyridylboronic acid with (4-carbamoylphenyl) boronic acid, provided the title compound hydrochloride salt as a yellow solid. LC-MS: [M+H⁺]=593.4. ¹H NMR (400 MHz, CD₃OD): δ 8.74 (br d, J=3.8 Hz, 1H), 8.50 (br d, J=8.0 Hz, 1H), 8.40 (d, J=5.6 Hz, 1H), 8.11 (br d, J=10.5 Hz, 1H), 8.05-7.97 (m, 1H), 8.01 (br d, J=8.0 Hz, 2H), 7.95-7.86 (m, 4H), 7.52 (dd, J=4.4, 8.3 Hz, 1H), 7.43 (br t, J=7.9 Hz, 1H), 5.33-5.22 (m, 1H), 3.97-3.89 (m, 1H), 3.73-3.63 (m, 1H), 3.40-3.33 (m, 2H), 2.89 (dt, J=3.1, 11.9 Hz, 1H), 2.17-2.07 (m, 1H), 2.02-1.83 (m, 2H), 1.48-1.26 (m, 1H).

### Synthesis of 2-fluoro-N-[2-(3-oxazol-2-ylphenyl) thieno [3, 2-c] pyridin-4-yl]-N-[(3R)-3-piperidyl]-4-(triazolo [4, 5-b] pyridin-3-yl) benzamide (Example 449)

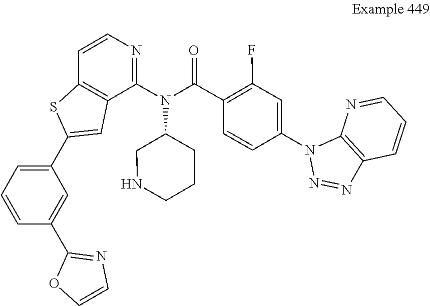 [SMILES: O=C(c1ccc(-n2nnc3cccnc32)cc1F)N(c1nccc2sc(-c3cccc(-c4ncco4)c3)cc12)[C@@H]1CCCNC1]

Example 446 was synthesized following the procedure for Example 161 by substituting 4-pyridylboronic acid with 2-[3-(4, 4, 5, 5-tetramethyl-1, 3, 2-dioxaborolan-2-yl) phenyl] oxazole, provided the title compound hydrochloride salt as a white solid. LC-MS: [M+H⁺]=617.4. ¹H NMR (400 MHZ, CD₃OD): δ 8.67 (br d, J=3.5 Hz, 1H), 8.49-8.36 (m, 3H), 8.14-8.04 (m, 3H), 8.00 (br d, J=7.9 Hz, 1H), 7.94-7.88 (m, 3H), 7.68-7.61 (m, 1H), 7.53-7.42 (m, 2H), 7.39 (s, 1H), 5.37-5.18 (m, 1H), 3.95 (br d, J=10.6 Hz, 1H), 3.70 (br t, J=11.7 Hz, 1H), 3.44-3.33 (m, 1H), 2.99-2.83 (m, 1H), 2.14 (br d, J=10.6 Hz, 1H), 2.05-1.84 (m, 2H), 1.57-1.36 (m, 1H).

### Synthesis of 2-fluoro-N-[(3R)-3-piperidyl]-N-[2-([1,2,4]triazolo[1,5-a]pyridin-2-yl)thieno[3,2-c]pyridin-4-yl]-4-(triazolo[4,5-b]pyridin-3-yl)benzamide (Example 450)

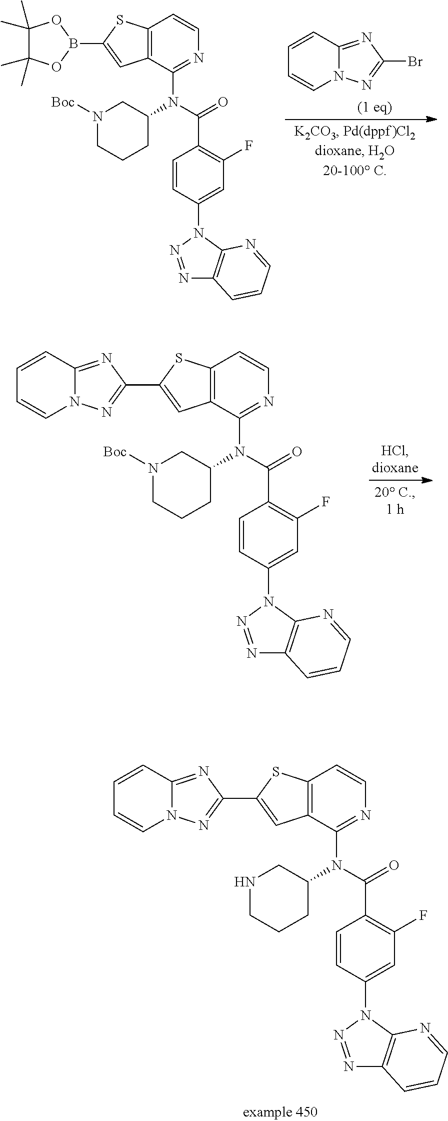 [SMILES: CC(C)(C)OC(=O)N1CCC[C@@H](N(C(=O)c2ccc(-n3nnc4cccnc43)cc2F)c2nccc3sc(B4OC(C)(C)C(C)(C)O4)cc23)C1 ; Brc1nc2ccccn2n1 ; CC(C)(C)OC(=O)N1CCC[C@@H](N(C(=O)c2ccc(-n3nnc4cccnc43)cc2F)c2nccc3sc(-c4nc5ccccn5n4)cc23)C1 ; O=C(c1ccc(-n2nnc3cccnc32)cc1F)N(c1nccc2sc(-c3nc4ccccn4n3)cc12)[C@@H]1CCCNC1]

Example 450 was prepared according to the procedure described for Example 415 by substituting 4-bromo-1-isobutyl-triazole with 2-bromo-[1,2,4]triazolo[1,5-a]pyridine, provided the title compound hydrochloride salt as an off-white solid. LC-MS: [M+H⁺]=591.20. ¹H NMR (400 MHZ, DMSO-d₆): δ 9.18-8.96 (m, 2H), 8.92-8.74 (m, 2H), 8.66 (br d, J=8.3 Hz, 1H), 8.52-8.40 (m, 1H), 8.29-8.10 (m, 2H), 8.08-7.85 (m, 3H), 7.78 (br t, J=7.9 Hz, 1H), 7.67-7.53 (m, 1H), 7.49-7.38 (m, 1H), 7.32 (t, J=6.6 Hz, 1H), 5.25-4.81 (m, 1H), 3.85-3.65 (m, 1H), 3.24-3.08 (m, 2H), 2.78-2.66 (m, 1H), 2.10-1.73 (m, 4H), 1.28-1.19 (m, 1H).

### Synthesis of 2-fluoro-N-[(3R)-3-piperidyl]-N-(2-pyrazin-2-ylthieno[3,2-c]pyridin-4-yl)-4-(triazolo[4,5-b]pyridin-3-yl)benzamide (Example 451)

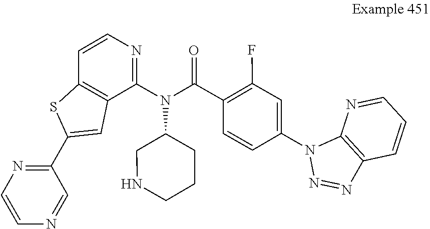 [SMILES: O=C(c1ccc(-n2nnc3cccnc32)cc1F)N(c1nccc2sc(-c3cnccn3)cc12)[C@@H]1CCCNC1]

Example 451 was synthesized following the procedure for Example 161 by substituting 4-pyridylboronic acid with 2-(4,4,5,5-tetramethyl-1,3,2-dioxaborolan-2-yl)pyrazine to afford the title compound hydrochloride salt as a white solid. LC-MS: [M+H⁺]=552.4. ¹H NMR (400 MHZ, CD₃OD): δ 9.39 (s, 1H), 8.73 (br d, J=3.9 Hz, 1H), 8.62 (br d, J=12.3 Hz, 2H), 8.49 (br d, J=8.5 Hz, 1H), 8.42 (br d, J=5.5 Hz, 1H), 8.36-8.23 (m, 1H), 8.15-7.88 (m, 3H), 7.60-7.40 (m, 2H), 5.36-5.21 (m, 1H), 3.95 (br d, J=13.2 Hz, 1H), 3.75-3.63 (m, 2H), 3.58-3.53 (m, 1H), 3.41-3.34 (m, 1H), 2.98-2.82 (m, 1H), 2.21-2.07 (m, 1H), 2.04-1.81 (m, 2H), 1.47-1.27 (m, 1H).

### Synthesis of N-[2-(3-carbamoylphenyl)thieno[3,2-c]pyridin-4-yl]-2-fluoro-N-[(3R)-3-piperidyl]-4-(triazolo[4,5-b]pyridin-3-yl)benzamide (Example 452)

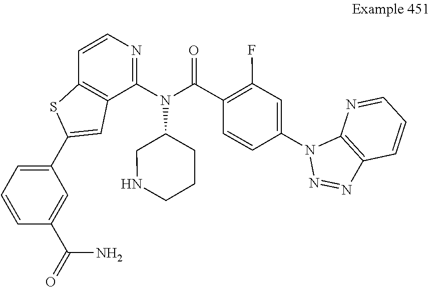 [SMILES: NC(=O)c1cccc(-c2cc3c(N(C(=O)c4ccc(-n5nnc6cccnc65)cc4F)[C@@H]4CCCNC4)nccc3s2)c1]

Example 451 was prepared according to the procedure described for Example 161 by substituting 4-pyridylboronic acid with (3-carbamoylphenyl)boronic acid, provided the title compound hydrochloride salt as an off-white solid. LC-MS: [M+H⁺]=593.3. ¹H NMR (400 MHz, CD₃OD): δ 8.73 (br d, J=3.9 Hz, 1H), 8.50 (br d, J=8.1 Hz, 1H), 8.39 (br d, J=5.4 Hz, 1H), 8.31 (s, 1H), 8.10 (br d, J=10.9 Hz, 1H), 8.04-7.86 (m, 5H), 7.62 (br t, J=7.7 Hz, 1H), 7.52 (dd, J=4.5, 8.3 Hz, 1H), 7.45 (br t, J=7.9 Hz, 1H), 5.36-5.21 (m, 1H), 3.94 (br d, J=11.9 Hz, 1H), 3.77-3.61 (m, 2H), 3.59-3.44 (m, 1H), 3.40-3.34 (m, 1H), 2.99-2.80 (m, 1H), 2.52-2.20 (m, 1H), 2.13 (br d, J=11.3 Hz, 1H), 2.01-1.86 (m, 2H), 1.49-1.35 (m, 1H), 1.35-1.25 (m, 1H).

### Synthesis of (R)-1-(4-(3H-[1,2,3]triazolo[4,5-b]pyridin-3-yl)-2-fluoro-N-(piperidin-3-yl)benzamido)isoquinoline-6-carboxamide (Example 453)

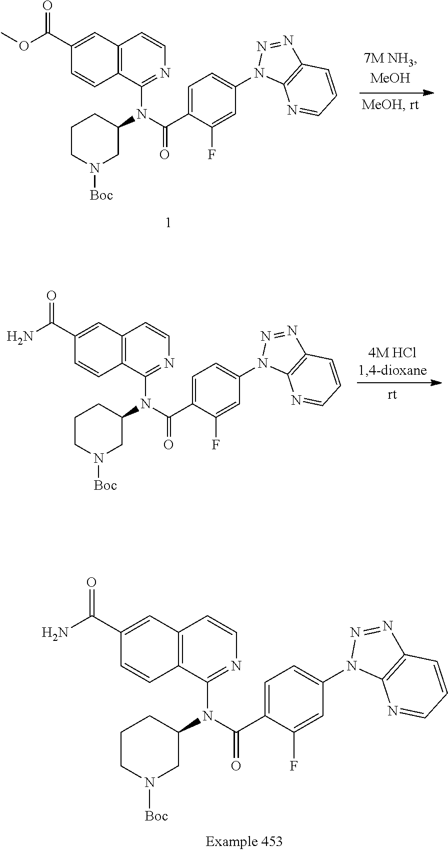 [SMILES: COC(=O)c1ccc2c(N(C(=O)c3ccc(-n4nnc5cccnc54)cc3F)[C@@H]3CCCN(C(=O)OC(C)(C)C)C3)nccc2c1 ; CC(C)(C)OC(=O)N1CCC[C@@H](N(C(=O)c2ccc(-n3nnc4cccnc43)cc2F)c2nccc3cc(C(N)=O)ccc23)C1 ; CC(C)(C)OC(=O)N1CCC[C@@H](N(C(=O)c2ccc(-n3nnc4cccnc43)cc2F)c2nccc3cc(C(N)=O)ccc23)C1]

### Step 1:

To a stirred solution of methyl (R)-1-(4-(3H-[1,2,3]triazolo[4,5-b]pyridin-3-yl)-N-(1-(tert-butoxycarbonyl)piperidin-3-yl)-2-fluorobenzamido) isoquinoline-6-carboxylate (50 mg, 0.080 mmol) in MeOH (2 mL) at room temperature was added a solution of ammonia in MeOH (2 mL, 0.080 mmol). The reaction was stirred for 16 h and then it was concentrated.residue The residue was further purified by Prep=HPLC (Condition: LUNA (250×21.2×5 μm), Mobile phase A: 0.1% formic acid in water (100%), Mobile phase B: 100% MeCN, Gradient: Time (min)/% B: 0/45, 10/70, Flow: 15 mL/min, Dilution: MeCN+water). The pure fractions were combined and dried by lyophilization to afford tert-butyl (R)-3-(4-(3H-[1,2,3]triazolo[4,5-b]pyridin-3-yl)-N-(6-carbamoylisoquinolin-1-yl)-2-fluorobenzamido)piperidine-1-carboxylate (20 mg, 0.032 mmol) as an off-white solid. LC-MS: [M+H⁺]=611.

### Step 2:

The title compound hydrochloride salt was obtained as an off-white solid by deprotection Method A used in Example 97. LC-MS: [M+H⁺]=511. ¹H NMR (CD₃OD, 400 MHz): δ 8.73 (d, J=4.8 Hz, 1H), 8.57 (d, J=9.6 Hz, 1H), 8.49 (d, J=8.4 Hz, 1H), 8.36-8.26 (m, 2H), 8.18-8.11 (m, 1H), 8.03 (d, J=9.6 Hz, 1H), 7.94-7.86 (m, 2H), 7.53-7.50 (m, 1H), 7.33-7.29 (m, 1H), 5.32-5.25 (m, 1H), 3.99-3.96 (m, 1H), 3.74-3.68 (m, 1H), 2.92-2.83 (m, 1H), 2.10-1.88 (m, 3H), 1.34-1.28 (m, 1H), 0.96-0.87 (m, 1H).

### Synthesis of 2-fluoro-N-[(3R)-3-piperidyl]-4-(triazolo[4,5-b]pyridin-3-yl)-N-[2-[6-(trifluoromethyl)-3-pyridyl]thieno[3,2-c]pyridin-4-yl]benzamide (Example 454)

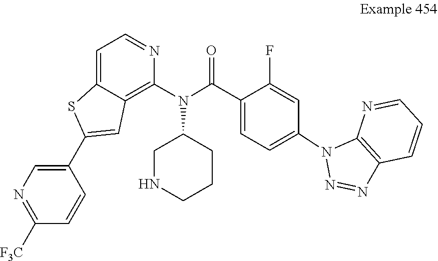 [SMILES: O=C(c1ccc(-n2nnc3cccnc32)cc1F)N(c1nccc2sc(-c3ccc(C(F)(F)F)nc3)cc12)[C@@H]1CCCNC1]

Example 454 was synthesized following the procedure for example 161 by substituting 4-pyridylboronic acid with [6-(trifluoromethyl)-3-pyridyl]boronic acid afforded the title compound as off white solid. LC-MS: [M+H⁺]=619.4. ¹H NMR (400 MHZ, DMSO-d₆): δ 9.42-9.30 (m, 1H), 9.27-8.94 (m, 2H), 8.79 (br d, J=3.9 Hz, 1H), 8.71-8.40 (m, 4H), 8.18-8.05 (m, 2H), 8.04-7.80 (m, 2H), 7.66-7.45 (m, 2H), 5.25-4.92 (m, 1H), 3.97-3.65 (m, 1H), 3.20 (br d, J=10.5 Hz, 1H), 2.84-2.65 (m, 1H), 2.41-1.61 (m, 4H), 1.37-1.03 (m, 1H).

### Synthesis of 2-fluoro-N-[(3R)-3-piperidyl]-N-(2-pyridazin-4-ylthieno[3,2-c]pyridin-4-yl)-4-(triazolo[4,5-b]pyridin-3-yl)benzamide (Example 455)

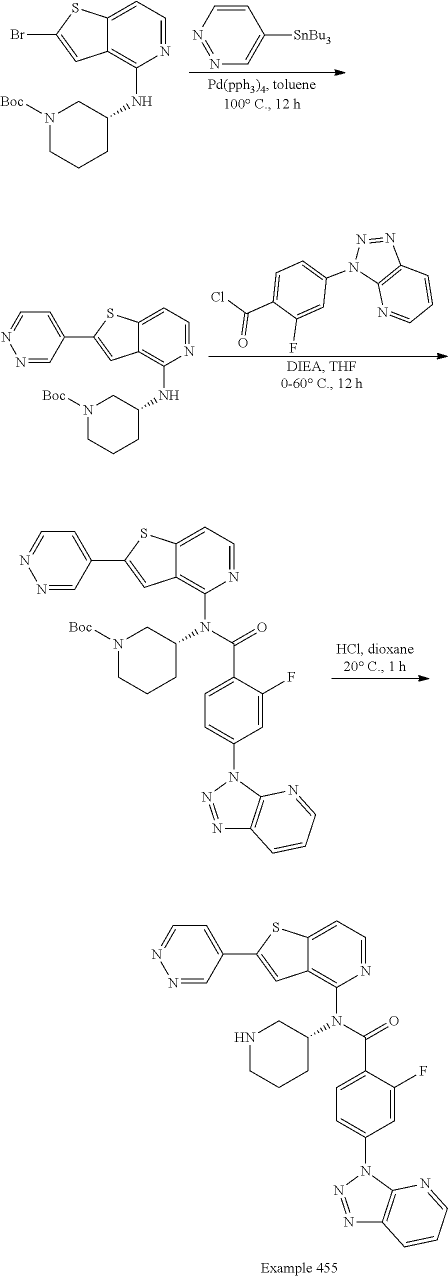

### Step 1:

A mixture of tributyl(pyridazin-4-yl)stannane (483.41 mg, 1.31 mmol), tert-butyl (3R)-3-[(2-bromothieno[3,2-c]pyridin-4-yl)amino]piperidine-1-carboxylate (200 mg, 436.53 μmol), Pd(PPh₃)₄ (50.44 mg, 43.65 μmol) in toluene (2 mL) was purged with N₂ for 3 times, and then the mixture was stirred at 100° C. for 12 h under N₂ atmosphere. The reaction mixture was diluted with water (5 mL) at 0° C., and extracted with ethyl acetate (3×15 mL). The combined organic layer was washed with brine (5 mL), dried over Na₂SO₄, filtered and concentrated. The residue was purified by prep-HPLC (neutral condition, column: Waters Xbridge Prep OBD C18 150*40 mm*10 μm; mobile phase: [H₂O (10 mM NH₄HCO₃)-MeCN]; gradient: 35%-70% B over 6.0 min), then pure fraction was lyophilized to afford tert-butyl (3R)-3-[(2-pyridazin-4-ylthieno[3,2-c]pyridin-4-yl)amino]piperidine-1-carboxylate (140 mg, 70%) as yellow solid.

### Steps 2-3:

Example 455 was prepared according to the procedure for Example 121 by substituting tert-butyl (3R)-3-[(2-pyridazin-4-ylthieno[3,2-c]pyridin-4-yl)amino]piperidine-1-carboxylate with tert-butyl (R)-3-((3-methylthieno[3,2-c]pyridin-4-yl)amino)piperidine-1-carboxylate, provided the title compound as an off-white solid. LC-MS: [M+H]=552.4. ¹H NMR (400 MHZ, DMSO-d₆): δ 9.97-9.87 (m, 1H), 9.48-9.13 (m, 3H), 8.79 (br d, J=3.5 Hz, 1H), 8.72-8.63 (m, 2H), 8.46 (br d, J=5.5 Hz, 1H), 8.15 (br d, J=5.4 Hz, 2H), 7.97 (br d, J=10.5 Hz, 1H), 7.89 (br d, J=8.4 Hz, 1H), 7.59 (br dd, J=4.8, 8.3 Hz, 2H), 5.20-5.01 (m, 1H), 3.82-3.70 (m, 1H), 3.41-3.29 (m, 1H), 3.19 (br d, J=10.0 Hz, 1H), 2.76-2.66 (m, 1H), 2.12 (br d, J=10.4 Hz, 1H), 1.94-1.72 (m, 3H), 1.26-1.09 (m, 1H).

### Synthesis of 2-fluoro-N-[(3R)-3-piperidyl]-N-(2-pyrimidin-4-ylthieno[3,2-c]pyridin-4-yl)-4-(triazolo[4,5-b]pyridin-3-yl)benzamide (Example 456)

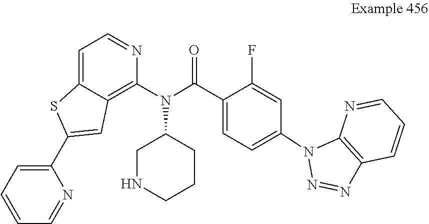 [SMILES: O=C(c1ccc(-n2nnc3cccnc32)cc1F)N(c1nccc2sc(-c3ccccn3)cc12)[C@@H]1CCCNC1]

Example 456 was synthesized following the procedure for Example 455 by substituting tributyl(pyridazin-4-yl)stannane with tributyl(pyrimidin-4-yl)stannane to afford the title compound hydrochloride salt as an off-white solid. LC-MS: [M+H]=552.4. ¹H NMR (400 MHZ, DMSO-d₆): δ 9.32-9.10 (m, 2H), 9.08-8.94 (m, 2H), 8.81-8.72 (m, 2H), 8.66 (br d, J=8.0 Hz, 1H), 8.58-8.38 (m, 2H), 8.10 (d, J=5.5 Hz, 1H), 7.97 (br d, J=10.5 Hz, 1H), 7.88 (br d, J=8.1 Hz, 1H), 7.65-7.50 (m, 2H), 5.24-4.92 (m, 1H), 3.86-3.72 (m, 1H), 3.40-3.28 (m, 1H), 3.19 (br d, J=10.5 Hz, 1H), 2.87-2.64 (m, 1H), 2.18-1.70 (m, 3H), 1.27-1.08 (m, 1H).

### Synthesis of N-[2-(2-aminopyrimidin-4-yl)thieno[3,2-c]pyridin-4-yl]-2-fluoro-N-[(3R)-3-piperidyl]-4-(triazolo[4,5-b]pyridin-3-yl)benzamide (Example 457)

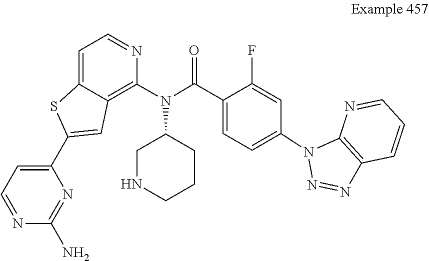 [SMILES: Nc1nccc(-c2cc3c(N(C(=O)c4ccc(-n5nnc6cccnc65)cc4F)[C@@H]4CCCNC4)nccc3s2)n1]

Example 457 was prepared following the procedure for Example 455 by substituting tributyl(pyridazin-4-yl)stannane with 4-tributylstannyl pyrimidin-2-amine, provided the title compound hydrochloride salt as a white solid. LC-MS: [M+H⁺]=567.3. ¹H NMR (400 MHz, CD₃OD): δ 8.77-8.62 (m, 2H), 8.51 (br d, J=5.6 Hz, 2H), 8.42 (br d, J=6.5 Hz, 1H), 8.09-7.94 (m, 3H), 7.81 (br d, J=5.4 Hz, 1H), 7.56-7.42 (m, 2H), 5.30 (br s, 1H), 3.96 (br d, J=9.8 Hz, 1H), 3.80-3.65 (m, 1H), 3.38 (br d, J=12.5 Hz, 1H), 2.91 (br s, 1H), 2.19-2.07 (m, 1H), 1.97 (br s, 2H), 1.53-1.24 (m, 2H).

### Synthesis of N-[2-[2-(dimethylamino)pyrimidin-4-yl]thieno[3,2-c]pyridin-4-yl]-2-fluoro-N-[(3R)-3-piperidyl]-4-(triazolo[4,5-b]pyridin-3-yl)benzamide (Example 458)

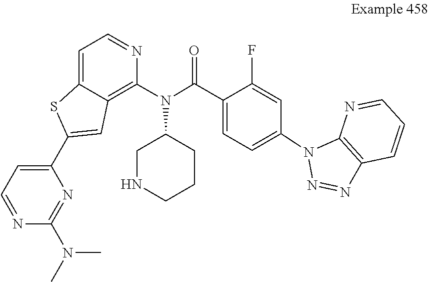 [SMILES: CN(C)c1nccc(-c2cc3c(N(C(=O)c4ccc(-n5nnc6cccnc65)cc4F)[C@@H]4CCCNC4)nccc3s2)n1]

Example 458 was synthesized following the procedure for Example 450 by substituting 2-bromo-[1,2,4]triazolo[1,5-a]pyridine with 4-chloro-N,N-dimethyl-pyrimidin-2-amine, provided the title compound hydrochloride salt as an off-white solid. LC-MS: [M+H⁺]=595.4. ¹H NMR (400 MHZ, DMSO-d₆): δ 9.31-8.92 (m, 2H), 8.79 (br d, J=3.6 Hz, 1H), 8.67 (br d, J=8.1 Hz, 1H), 8.59-8.50 (m, 2H), 8.42 (br d, J=5.3 Hz, 1H), 8.05 (br d, J=5.4 Hz, 1H), 8.01-7.93 (m, 1H), 7.88 (br d, J=8.5 Hz, 1H), 7.62-7.48 (m, 3H), 5.27-4.88 (m, 1H), 3.88-3.69 (m, 1H), 3.37-3.29 (m, 1H), 3.26-3.20 (m, 1H), 3.15 (s, 6H), 2.80-2.66 (m, 1H), 2.08 (br d, J=9.1 Hz, 1H), 2.12-1.74 (m, 2H), 1.23-1.07 (m, 1H).

### Synthesis of 2-fluoro-N-[2-(2-oxo-1H-pyridin-4-yl)thieno[3,2-c]pyridin-4-yl]-N-[(3R)-3-piperidyl]-4-(triazolo[4,5-b] pyridin-3-yl)benzamide (Example 459)

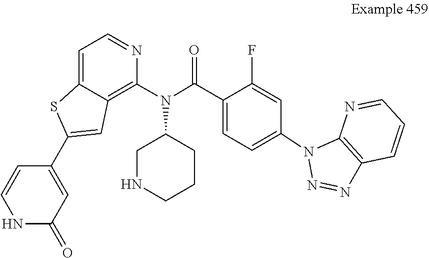 [SMILES: O=C(c1ccc(-n2nnc3cccnc32)cc1F)N(c1nccc2sc(-c3cc[nH]c(=O)c3)cc12)[C@@H]1CCCNC1]

Example 459 was synthesized following the procedure for Example 161 by substituting 4-pyridylboronic acid with (2-oxo-1H-pyridin-4-yl)boronic acid, provided the title compound hydrochloride salt as an off-white solid. LC-MS: [M+H⁺]=567.3. ¹H NMR (400 MHZ, CD₃OD): δ 8.75 (br d, J=3.9 Hz, 1H), 8.56-8.40 (m, 2H), 8.16-7.90 (m, 4H), 7.65-7.51 (m, 2H), 7.50-7.37 (m, 1H), 6.95-6.82 (m, 2H), 5.29 (br d, J=10.5 Hz, 1H), 3.99-3.84 (m, 1H), 3.70 (br t, J=11.9 Hz, 1H), 3.38 (br s, 1H), 2.98-2.83 (m, 1H), 2.19-2.05 (m, 1H), 2.02-1.84 (m, 2H), 1.49-1.27 (m, 1H)

### Synthesis of 2-fluoro-N-[2-(2-methoxy-4-pyridyl)thieno[3,2-c]pyridin-4-yl]-N-[(3R)-3-piperidyl]-4-(triazolo[4,5-b]pyridin-3-yl)benzamide (Example 460)

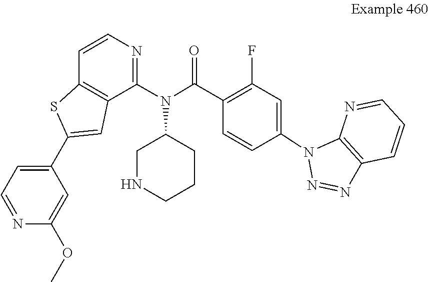 [SMILES: COc1cc(-c2cc3c(N(C(=O)c4ccc(-n5nnc6cccnc65)cc4F)[C@@H]4CCCNC4)nccc3s2)ccn1]

Example 460 was synthesized following the procedure for Example 161 by substituting 4-pyridylboronic acid with (2-methoxy-4-pyridyl) boronic acid, provided the title compound hydrochloride salt as a white solid. LC-MS: [M+H⁺]=581.3. ¹H NMR (400 MHz, CD₃OD): δ 8.72 (br s, 1H), 8.48 (br d, J=8.3 Hz, 1H), 8.38 (br s, 1H), 8.23 (d, J=5.4 Hz, 1H), 8.10-7.91 (m, 3H), 7.86 (br d, J=4.1 Hz, 1H), 7.51 (br dd, J=4.3, 7.1 Hz, 1H), 7.44-7.28 (m, 2H), 7.11 (s, 1H), 4.93 (br s, 1H), 3.97 (s, 3H), 3.64-3.35 (m, 1H), 3.29-3.10 (m, 1H), 2.94 (br d, J=11.6 Hz, 1H), 2.39 (br s, 1H), 2.08-1.93 (m, 1H), 1.89-1.63 (m, 2H), 1.38-1.16 (m, 1H).

### Synthesis of N-[2-(2-ethyl-4-pyridyl)thieno[3,2-c]pyridin-4-yl]-2-fluoro-N-[(3R)-3-piperidyl]-4-(triazolo[4,5-b]pyridin-3-yl)benzamide (Example 461)

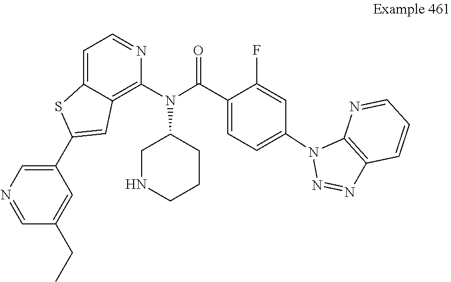 [SMILES: CCc1cncc(-c2cc3c(N(C(=O)c4ccc(-n5nnc6cccnc65)cc4F)[C@@H]4CCCNC4)nccc3s2)c1]

Example 461 was synthesized following the procedure for Example 161 by substituting 4-pyridylboronic acid with 2-ethyl-4-(4,4,5,5-tetramethyl-1,3,2-dioxaborolan-2-yl)pyridine, provided the title compound hydrochloride salt as an off-white solid. LC-MS: [M+H⁺]=579.3. ¹H NMR (400 MHZ, CD₃OD): δ 8.77 (br d, J=6.1 Hz, 1H), 8.71 (br s, 1H), 8.62 (br s, 1H), 8.55-8.45 (m, 2H), 8.41-8.29 (m, 2H), 8.01 (br d, J=4.4 Hz, 3H), 7.51 (br s, 2H), 5.31 (br s, 1H), 4.03-3.68 (m, 2H), 3.39 (br d, J=11.8 Hz, 1H), 3.18 (q, J=7.4 Hz, 2H), 2.91 (br s, 1H), 2.16 (br d, J=4.1 Hz, 1H), 2.06-1.89 (m, 2H), 1.51 (t, J=7.5 Hz, 4H).

### Synthesis of 2-fluoro-N-[(3R)-3-piperidyl]-4-(triazolo[4,5-b]pyridin-3-yl)-N-[2-[2-(trifluoromethyl)-4-pyridyl]thieno[3,2-c]pyridin-4-yl]benzamide (Example 462)

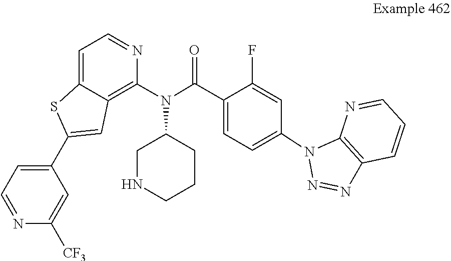 [SMILES: O=C(c1ccc(-n2nnc3cccnc32)cc1F)N(c1nccc2sc(-c3ccnc(C(F)(F)F)c3)cc12)[C@@H]1CCCNC1]

Example 462 was synthesized following the procedure for Example 161 by substituting 4-pyridylboronic acid with 4-(4,4,5,5-tetramethyl-1,3,2-dioxaborolan-2-yl)-2-(trifluoromethyl)pyridine, provided the title compound hydrochloride salt as an-off white solid. LC-MS: [M+H]=619.3. ¹H NMR (400 MHZ, DMSO-d₆): δ 8.97-8.81 (m, 3H), 8.80-8.73 (m, 2H), 8.71-8.64 (m, 1H), 8.57-8.41 (m, 2H), 8.17-8.05 (m, 2H), 7.98 (br d, J=9.6 Hz, 1H), 7.88 (br d, J=7.8 Hz, 1H), 7.65-7.49 (m, 2H), 5.24-4.96 (m, 1H), 3.90-3.72 (m, 1H), 3.25-3.15 (m, 1H), 2.83-2.64 (m, 2H), 2.20-1.68 (m, 3H), 1.21-1.04 (m, 1H).

### Synthesis of tert-butyl (3R)-3-[[2-[2-(1-cyano-1-methyl-ethyl)-4-pyridyl]thieno[3,2-c]pyridin-4-yl]-[2-fluoro-4-(triazolo[4,5-b]pyridin-3-yl)benzoyl] amino]piperidine-1-carboxylate (Example 463)

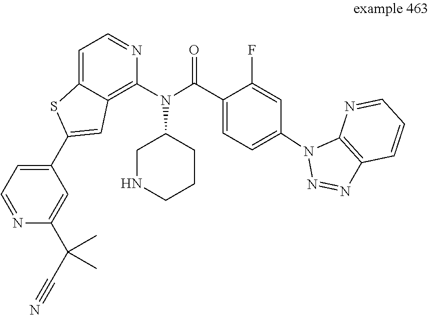 [SMILES: CC(C)(C#N)c1cc(-c2cc3c(N(C(=O)c4ccc(-n5nnc6cccnc65)cc4F)[C@@H]4CCCNC4)nccc3s2)ccn1]

Example 463 was synthesized following the procedure for Example 161 by substituting 4-pyridylboronic acid with 2-methyl-2-[4-(4,4,5,5-tetramethyl-1,3,2-dioxaborolan-2-yl)-2-pyridyl]propanenitrile, provided the title compound hydrochloride salt as a white solid. LC-MS: [M+H⁺]=618.4. ¹H NMR (400 MHZ, CD₃OD): δ 8.76-8.65 (m, 2H), 8.53-8.37 (m, 2H), 8.17-8.02 (m, 2H), 8.02-7.85 (m, 3H), 7.74 (br d, J=3.6 Hz, 1H), 7.56-7.34 (m, 2H), 4.65-4.48 (m, 2H), 3.66-3.50 (m, 1H), 3.28-3.14 (m, 1H), 2.95 (br d, J=11.0 Hz, 1H), 2.54-2.34 (m, 1H), 2.14-1.95 (m, 1H), 1.85 (s, 6H), 1.77-1.59 (m, 2H), 1.37-1.16 (m, 1H).

### Synthesis of 2-fluoro-N-(2-phenylthieno[3,2-c]pyridin-4-yl)-N-[(3R)-3-piperidyl]-4-(triazolo[4,5-b]pyridin-3-yl)benzamide (Example 464)

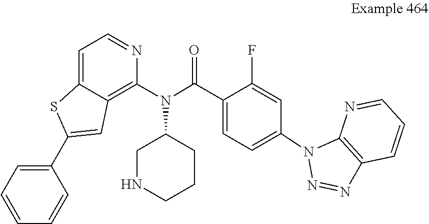 [SMILES: O=C(c1ccc(-n2nnc3cccnc32)cc1F)N(c1nccc2sc(-c3ccccc3)cc12)[C@@H]1CCCNC1]

Example 464 was prepared according to the procedure described for Example 161 by substituting 4-pyridylboronic acid with 4,4,5,5-tetramethyl-2-phenyl-1,3,2-dioxaborolane, provided the title compound hydrochloride salt as a white solid. LC-MS: [M+H⁺]=550.4. ¹H NMR (400 MHZ, CD₃OD): δ 8.74 (br d, J=3.9 Hz, 1H), 8.50 (br d, J=8.3 Hz, 1H), 8.39 (br d, J=5.4 Hz, 1H), 8.12 (br d, J=10.8 Hz, 1H), 8.01 (br d, J=8.0 Hz, 1H), 7.89 (br d, J=5.3 Hz, 1H), 7.86-7.73 (m, 3H), 7.65-7.32 (m, 5H), 5.40-5.18 (m, 1H), 3.95 (br d, J=11.1 Hz, 1H), 3.69 (br t, J=11.8 Hz, 1H), 3.38 (br d, J=12.5 Hz, 1H), 3.03-2.82 (m, 1H), 2.13 (br d, J=10.9 Hz, 1H), 2.06-1.82 (m, 2H), 1.56-1.27 (m, 1H).

### Synthesis of 2-fluoro-N-[(3R)-3-piperidyl]-N-[2-(3-pyridylmethyl)thieno[3,2-c]pyridin-4-yl]-4-(triazolo[4,5-b]pyridin-3-yl)benzamide (Example 465)

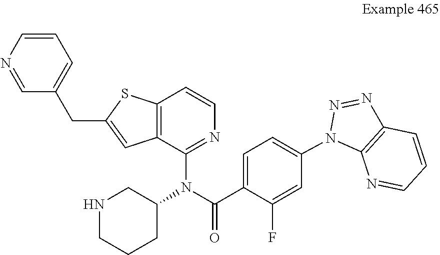 [SMILES: O=C(c1ccc(-n2nnc3cccnc32)cc1F)N(c1nccc2sc(Cc3cccnc3)cc12)[C@@H]1CCCNC1]

Example 465 was synthesized following the procedure for Example 450 by substituting 2-bromo-[1,2,4]triazolo[1,5-a]pyridine with 3-(bromomethyl)pyridine; hydrobromide, provided the title compound hydrochloride salt as an off-white solid. LC-MS: [M+H]=565.3. ¹H NMR (400 MHZ, DMSO-d₆): δ 9.29-8.95 (m, 2H), 8.90-8.81 (m, 2H), 8.76-8.67 (m, 2H), 8.37-8.29 (m, 1H), 8.22-8.12 (m, 1H), 8.00-7.91 (m, 2H), 7.89-7.77 (m, 2H), 7.63 (dd, J=4.4, 8.3 Hz, 1H), 7.55-7.35 (m, 2H), 5.19-4.86 (m, 1H), 4.53-4.40 (m, 2H), 3.77-3.72 (m, 1H), 3.23-3.15 (m, 2H), 2.75-2.67 (m, 1H), 1.93-1.73 (m, 3H), 1.20-1.02 (m, 1H).

### Synthesis of N-(2-benzylthieno[3,2-c]pyridin-4-yl)-2-fluoro-N-[(3R)-3-piperidyl]-4-(triazolo[4,5-b]pyridin-3-yl)benzamide (Example 466)

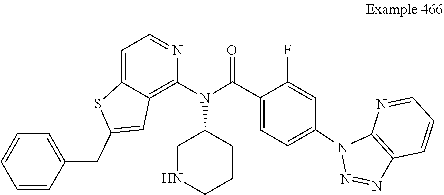 [SMILES: O=C(c1ccc(-n2nnc3cccnc32)cc1F)N(c1nccc2sc(Cc3ccccc3)cc12)[C@@H]1CCCNC1]

Example 466 was prepared according to the procedure described for Example 161 by substituting 4-pyridylboronic acid with 2-benzyl-4,4,5,5-tetramethyl-1,3,2-dioxaborolane, provided the title compound hydrochloride salt as off white solid. LC-MS: [M+H⁺]=564.4. ¹H NMR (400 MHZ, DMSO-d₆): δ 8.87 (br d, J=3.9 Hz, 3H), 8.71 (br d, J=8.6 Hz, 1H), 8.37-8.28 (m, 1H), 8.07-7.96 (m, 1H), 7.94-7.84 (m, 2H), 7.64 (br dd, J=4.6, 8.0 Hz, 1H), 7.43-7.21 (m, 7H), 5.22-4.84 (m, 1H), 4.38-4.19 (m, 2H), 3.79-3.66 (m, 1H), 3.20 (br d, J=11.9 Hz, 1H), 2.94-2.64 (m, 2H), 1.99-1.76 (m, 3H), 1.25-1.06 (m, 1H).

### Synthesis of 2-fluoro-N-[2-(3-formylphenyl)thieno[3,2-c]pyridin-4-yl]-N-[(3R)-3-piperidyl]-4-(triazolo[4,5-b]pyridin-3-yl)benzamide (Example 467)

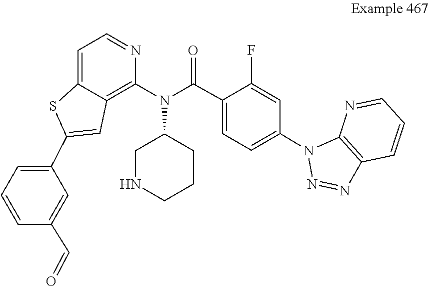 [SMILES: O=Cc1cccc(-c2cc3c(N(C(=O)c4ccc(-n5nnc6cccnc65)cc4F)[C@@H]4CCCNC4)nccc3s2)c1]

Example 467 was synthesized following the procedure for example 161 by substituting 4-pyridylboronic acid with (3-formylphenyl) boronic acid, provided the title compound hydrochloride salt as off white solid. LC-MS: [M+H⁺]=578.4. ¹H NMR (400 MHz, DMSO-d₆): δ 10.14 (br s, 1H), 8.78 (br d, J=2.5 Hz, 1H), 8.71-8.60 (m, 1H), 8.46-8.32 (m, 2H), 8.23 (br s, 2H), 8.06-7.93 (m, 3H), 7.89-7.75 (m, 2H), 7.63-7.47 (m, 2H), 4.87-4.51 (m, 1H), 2.99-2.73 (m, 2H), 2.25-2.08 (m, 2H), 2.05-1.92 (m, 1H), 1.78-1.38 (m, 3H), 1.10-0.77 (m, 1H).

### Synthesis of (R)-2-fluoro-N-(3-methylthieno[3,2-c]pyridin-4-yl)-N-(piperidin-3-yl)-4-(pyrimidin-2-ylamino)benzamide (Example 468)

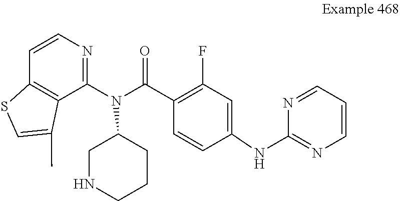 [SMILES: Cc1csc2ccnc(N(C(=O)c3ccc(Nc4ncccn4)cc3F)[C@@H]3CCCNC3)c12]

Example 468 was prepared according to the procedure for Example 119 by substituting tert-butyl (R)-3-((1-methyl-1H-pyrazolo[3,4-c]pyridin-7-yl)amino)piperidine-1-carboxylate with tert-butyl (R)-3-((3-methylthieno[3,2-c]pyridin-4-yl)amino)piperidine-1-carboxylate, provided the title compound hydrochloride salt as an off-white solid. LC-MS: [M−H]=463.2. ¹H NMR (400 MHZ, DMSO-d₆): δ ppm 9.89 (s, 1H), 9.27-8.94 (m, 2H), 8.49 (d, J=4.9 Hz, 2H), 8.43-8.34 (m, 1H), 8.07-7.99 (m, 1H), 7.67-7.50 (m, 2H), 7.09-7.01 (m, 1H), 6.91 (t, J=4.8 Hz, 1H), 6.83-6.74 (m, 1H), 4.99-4.66 (m, 1H), 3.71 (br s, 1H), 3.30-3.16 (m, 2H), 2.78-2.66 (m, 1H), 2.47 (s, 3H), 1.86-1.60 (m, 3H), 1.15-0.96 (m, 1H).

### Synthesis of (R)-2-fluoro-N-(6-fluoroisoquinolin-1-yl)-4-(1-methyl-1H-1,2,3-triazol-4-yl)-N-(piperidin-3-yl)benzamide (Example 469)

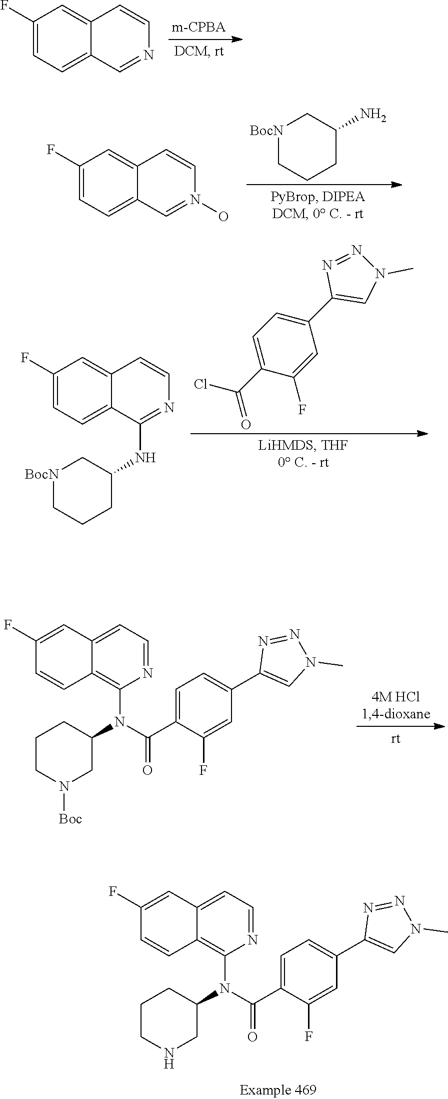

To a stirred solution of 6-fluoroisoquinoline (1 g, 6.80 mmol) in DCM (5 mL) was added m-CPBA (5.86 g, 34.0 mmol) at 0° C. and stirred at 25° C. for 4 h. The reaction mixture was diluted with ice cold water (50 mL) and extracted with DCM (2×50 mL). The combined organic layers were dried over Na₂SO₄ and concentrated under reduced pressure to afford 6-fluoroisoquinoline 2-oxide (450 mg, 40%) as an off-white solid. LC-MS: [M+H⁺]=164.1.

### Step 2:

To a stirred solution of 6-fluoroisoquinoline 2-oxide (450 g, 2.76 mmol) & tert-butyl (R)-3-aminopiperidine-1-carboxylate (663 mg, 3.31 mmol) in DCM (5 mL) was added DIPEA (1.927 mL, 11.03 mmol) and PyBroP (1.543 g, 3.31 mmol) at room temperature and was stirred for 4 h. The reaction mixture was quenched with saturated aq. NaHCO₃ solution (20 mL) and extracted with EtOAc (30 mL). The organic layer was dried over Na₂SO₄ and concentrated under reduced pressure. The residue was purified by Combi-flash using 50% EtOAc in hexane first and then further purified by Prep-HPLC (Conditions: Luna OMEGA C-18 (250×21.2×5 μm), Mobile phase A: 0.1% formic acid in water, Mobile phase B: MeCN (100%), Time (min)/% B: 0/10, 2/10, 10/60, MeCN+H₂O). Pure fractions were combined and concentrated to afford tert-butyl (R)-3-((6-fluoroisoquinolin-1-yl)amino)piperidine-1-carboxylate (300 mg) as an off-white solid. LC-MS: [M+H⁺]=346.2.

### Step 3:

To a solution of tert-butyl (R)-3-((6-fluoroisoquinolin-1-yl)amino) piperidine-1-carboxylate (100 mg, 0.290 mmol) in THF (10 mL) at 0° C. was added LiHMDS in THF (145 mg, 0.869 mmol) and stirred for 10 min and followed by addition of 2-fluoro-4-(1-methyl-1H-1,2,3-triazol-4-yl)benzoyl chloride (139 mg, 0.579 mmol) in THF (2 mL). The reaction was stirred for 1 h before it was quenched with ice cold water (10 mL) and extracted with EtOAc (2×20 mL). The combined organic layer was dried over Na₂SO₄ and concentrated under reduced pressure. The residue was further purified by Prep-HPLC (Conditions: X-SELECT C-18 (250×19×5 μm), Mobile phase A: 0.1% formic acid in water (100%), Mobile phase B: 100% MeCN, Gradient: 0/60, 10/80, Flow: 15 mL/min, Dilution: MeCN+THF). Pure fractions were combined and dried by lyophilization to afford tert-butyl (R)-3-(2-fluoro-N-(6-fluoroisoquinolin-1-yl)-4-(1-methyl-1H-1,2,3-triazol-4-yl)benzamido)piperidine-1-carboxylate (28 mg, 99%) as an off-white solid. LC-MS: [M+H⁺]=549.45.

### Step 4:

The title compound hydrochloride salt was obtained as an off-white solid by deprotection Method A used in Example 97. LC-MS: [M+H⁺]=449.2. ¹H NMR (CD₃OD, 400 MHZ): δ 8.43 (d, J=6.0 Hz, 1H), 8.25-8.22 (m, 1H), 8.18 (s, 1H), 7.73 (d, J=5.6 Hz, 1H), 7.59-7.49 (m, 2H), 7.28-7.26 (m, 1H), 7.22-7.20 (m, 1H), 7.09-7.06 (m, 1H), 5.27-5.21 (m, 1H), 4.06 (s, 3H), 3.94-3.91 (m, 1H), 3.71-3.65 (m, 1H), 2.88-2.84 (m, 1H), 2.03-1.97 (m, 1H), 1.91-1.86 (m, 2H), 1.39-1.23 (m, 1H), 0.94-0.91 (m, 1H).

### Synthesis of N-(6-chloro-1-isoquinolyl)-2-fluoro-4-(1-methyltriazol-4-yl)-N-[(3R)-3-piperidyl]benzamide (Example 470)

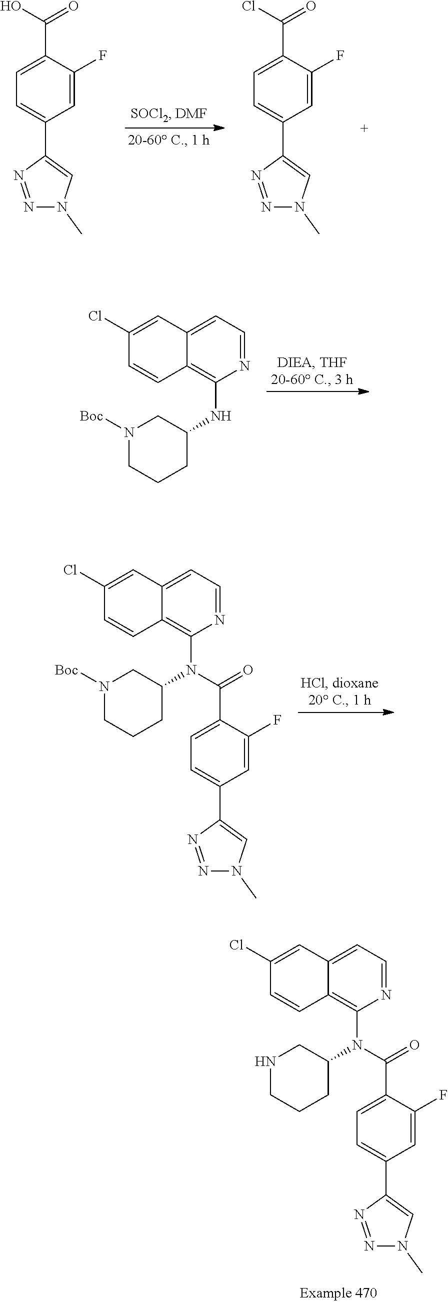 [SMILES: Cn1cc(-c2ccc(C(=O)O)c(F)c2)nn1 ; Cn1cc(-c2ccc(C(=O)Cl)c(F)c2)nn1 ; CC(C)(C)OC(=O)N1CCC[C@@H](Nc2nccc3cc(Cl)ccc23)C1 ; Cn1cc(-c2ccc(C(=O)N(c3nccc4cc(Cl)ccc34)[C@@H]3CCCN(C(=O)OC(C)(C)C)C3)c(F)c2)nn1 ; Cn1cc(-c2ccc(C(=O)N(c3nccc4cc(Cl)ccc34)[C@@H]3CCCNC3)c(F)c2)nn1]

### Step 1:

To a solution of 2-fluoro-4-(1-methyltriazol-4-yl)benzoic acid (6 g, 24.41 mmol) in SOCl₂ (50 mL) was added DMF (17.84 mg, 244.14 μmol) in order at 20° C. under N₂. Then the mixture was stirred at 60° C. for 1 hour. The reaction mixture was concentrated in vacuum to give 2-fluoro-4-(1-methyltriazol-4-yl)benzoyl chloride (6 g, 92%) as light yellow solid.

### Step 2:

To a solution of tert-butyl (3R)-3-[(6-chloro-1-isoquinolyl)amino]piperidine-1-carboxylate (4 g, 10.50 mmol) in THF (80 mL) was added DIEA (6.79 g, 52.51 mmol) and 2-fluoro-4-(1-methyltriazol-4-yl)benzoyl chloride (5.59 g, 21.00 mmol) in order at 20° C. under N₂. The mixture was stirred at 60° C. for 3 h and then was concentrated. The residue was diluted with water (100 mL) and extracted with ethyl acetate (3×50 mL). The combined organic phase was washed with brine (200 mL), dried with anhydrous Na₂SO₄, concentrated. The residue was purified by silica column chromatography (SiO₂, Petroleum ether/Tetrahydrofuran=70/30 to 50/50) and concentrated in vacuum to give tert-butyl (3R)-3-[(6-chloro-1-isoquinolyl)-[2-fluoro-4-(1-methyltriazol-4-yl)benzoyl]amino]piperidine-1-carboxylate (4.5 g, 68%) as light-yellow oil.

### Step 3:

The title compound hydrochloride salt was obtained as an off-white solid by deprotection Method A used in Example 97. LC-MS: [M+H⁺]=465.2. ¹H NMR (400 MHZ, DMSO-d₆): δ 9.44-9.18 (m, 1H), 9.08-8.91 (m, 1H), 8.55-8.40 (m, 2H), 8.24-8.01 (m, 2H), 7.85-7.66 (m, 2H), 7.40-7.21 (m, 2H), 7.16-7.01 (m, 1H), 5.21-4.72 (m, 1H), 4.00 (s, 3H), 3.72 (br d, J=11.5 Hz, 1H), 3.52-3.31 (m, 1H), 3.17 (br d, J=11.8 Hz, 1H), 2.83-2.61 (m, 1H), 2.33-2.02 (m, 1H), 1.98-1.68 (m, 3H), 1.17-0.97 (m, 1H).

### Synthesis of (R)-2-fluoro-N-(6-methoxyisoquinolin-1-yl)-4-(1-methyl-1H-1,2,3-triazol-4-yl)-N-(piperidin-3-yl)benzamide (Example 471)

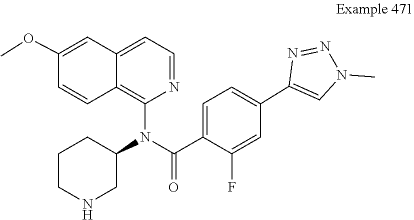

Example 495 was synthesized following the procedure for Example 469 by substituting 6-fluoroisoquinoline with 6-methoxyisoquinoline, provided the title compound hydrochloride salt as an off-white solid. LC-MS: [M+H⁺]=461.25.

1H NMR (CD₃OD, 400 MHZ): δ 8.34-8.32 (m, 1H), 8.20-8.18 (m, 1H), 8.12-8.04 (m, 1H), 7.71-7.70 (m, 1H), 7.44-7.27 (m, 3H), 7.23-7.22 (m, 1H), 7.15-7.11 (m, 1H), 5.22-5.19 (m, 1H), 4.17 (s, 3H), 3.93-3.90 (m, 1H), 3.89 (s, 3H), 3.68-3.65 (m, 1H), 3.34-3.32 (m, 1H), 2.89-2.86 (m, 1H), 2.03-1.87 (m, 3H), 1.33-1.28 (m, 1H).

### Synthesis of (R)—N-(6-cyanoisoquinolin-1-yl)-2-fluoro-4-(1-methyl-1H-1,2,3-triazol-4-yl)-N-(piperidin-3-yl)benzamide (Example 472)

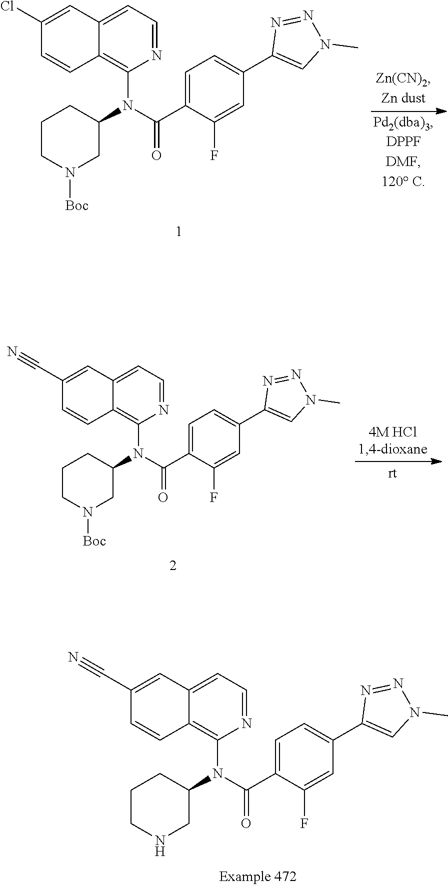 [SMILES: Cn1cc(-c2ccc(C(=O)N(c3nccc4cc(Cl)ccc34)[C@@H]3CCCN(C(=O)OC(C)(C)C)C3)c(F)c2)nn1 ; Cn1cc(-c2ccc(C(=O)N(c3nccc4cc(C#N)ccc34)[C@@H]3CCCN(C(=O)OC(C)(C)C)C3)c(F)c2)nn1 ; Cn1cc(-c2ccc(C(=O)N(c3nccc4cc(C#N)ccc34)[C@@H]3CCCNC3)c(F)c2)nn1]

### Step 1:

To a stirred solution of tert-butyl (R)-3-(N-(6-bromoisoquinolin-1-yl)-2-fluoro-4-(1-methyl-1H-1,2,3-triazol-4-yl)benzamido)piperidine-1-carboxylate (50 mg, 0.082 mmol) in DMF (1 mL) at room temperature was added zinc(II) cyanide (9.63 mg, 0.082 mmol), dppf (45.5 mg, 0.082 mmol) and zinc (5.36 mg, 0.082 mmol). The reaction mixture was degassed with nitrogen for 10 mins. Then Pd₂(dba)₃ (75 mg, 0.082 mmol) was added to the reaction mixture and stirred at 120° C. for 2 h. The reaction mixture was cooled to room temperature, diluted with water (10 mL) and extracted with EtOAc (2×50 mL). The combined organic layer was dried over Na₂SO₄ and concentrated. The residue was purified by Combi-flash eluting with 50% EtOAc in hexane first and then was further purified by Prep-HPLC (Condition: LUNA (250×21.2×5 μm), Mobile phase A: 0.1% formic acid in water (100%), Mobile phase B: 100% MeCN, Gradient: Time (min)/% B: 0/45, 10/70, Flow: 15 mL/min, Dilution: MeCN+water). The pure fractions were combined and dried by lyophilization to afford tert-butyl (R)-3-(N-(6-cyanoisoquinolin-1-yl)-2-fluoro-4-(1-methyl-1H-1,2,3-triazol-4-yl)benzamido)piperidine-1-carboxylate (22 mg, 62%) as an off-white solid.

### Step 2:

The title compound hydrochloride salt was obtained as an off-white solid by deprotection Method A used in Example 97. LC-MS: [M+H⁺]=456.2. ¹H NMR (CD₃OD, 400 MHZ): δ 8.59 (d, J=5.6 Hz, 1H), 8.40 (s, 1H), 8.31 (d, J=8.8 Hz, 1H), 8.15 (s, 1H), 7.93-7.85 (m, 2H), 7.27-7.20 (m, 2H), 7.08-7.05 (m, 1H), 5.26-5.23 (m, 1H), 4.05 (s, 3H), 3.95-3.92 (m, 1H), 3.72-3.65 (m, 1H), 2.88-2.81 (m, 1H), 1.99-1.89 (m, 3H), 1.39-1.20 (m, 1H), 0.96-0.89 (m, 1H).

### Synthesis of (R)-4-((1,3,5-triazin-2-yl)amino)-2-fluoro-N-(8-methylisoquinolin-1-yl)-N-(piperidin-3-yl)benzamide (Example 473)

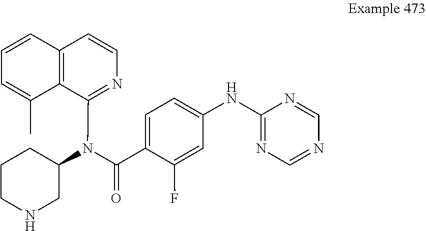 [SMILES: Cc1cccc2ccnc(N(C(=O)c3ccc(Nc4ncncn4)cc3F)[C@@H]3CCCNC3)c12]

Example 473 was synthesized following the procedure for Example 131 by substituting 2-aminopyrimidine with 1,3,5-triazin-2-amine, provided the title compound hydrochloride salt as an off-white solid. LC-MS: [M+H⁺]=458.2. ¹H NMR (CD₃OD, 400 MHz): δ 8.76 (s, 2H), 8.43-8.39 (m, 1H), 7.74-7.70 (m, 1H), 7.67-7.63 (m, 1H), 7.52-7.39 (m, 3H), 6.93 (dd, J=2 Hz, J=8.8 Hz, 1H), 6.81-6.77 (m, 1H), 5.08-5.05 (m, 1H), 3.98-3.96 (m, 1H), 3.83-3.77 (m, 1H), 3.48-3.38 (m, 1H), 3.02-2.92 (m, 1H), 2.81 (s, 3H), 2.71-2.67 (m, 1H), 1.98-1.95 (m, 2H), 1.57-1.44 (m, 1H).

### Synthesis of (R)-2-fluoro-N-(6-fluoro-8-methylisoquinolin-1-yl)-4-(1-methyl-1H-1,2,3-triazol-4-yl)-N-(piperidin-3-yl)benzamide (Example 474)

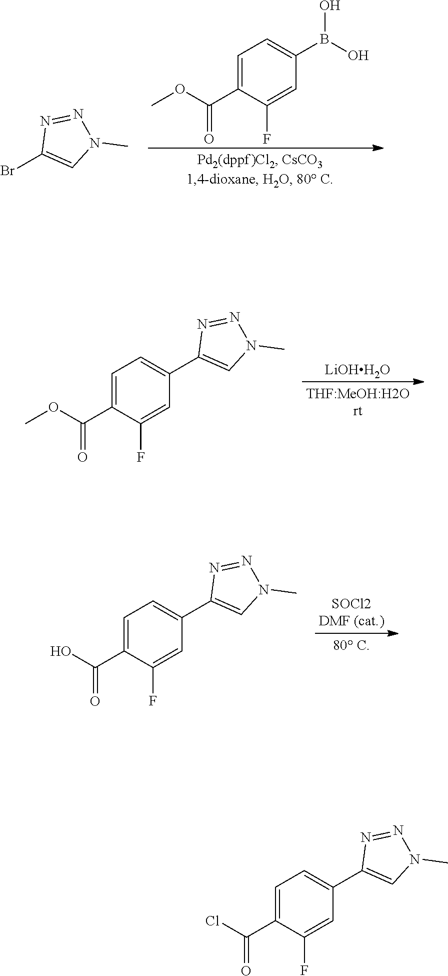

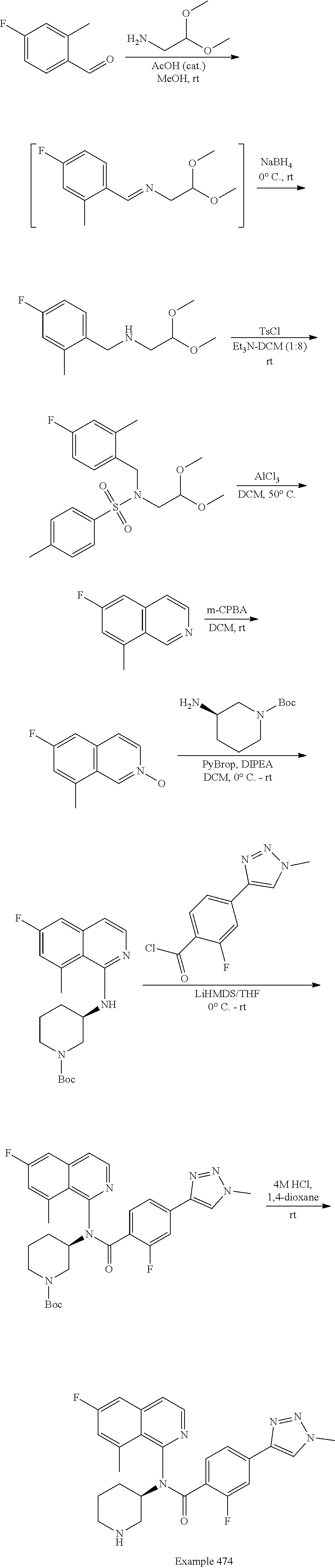

### Step 1:

To a solution of 4-bromo-1-methyl-1H-1,2,3-triazole (7 g, 43.20 mmol) in 1,4-dioxane (150 mL) at room temperature was added Cs₂CO₃ (42.2 g, 129.6 mmol), (3-fluoro-4-methoxycarbonyl)phenyl)boronic acid (15.4 g, 77.77 mmol) and H₂O (14 mL) and the reaction mixture was degassed with nitrogen for 5 min and followed by addition of Pd₂(dppf)Cl₂ (1.8 g, 2.16 mmol). The reaction mixture was stirred at 80° C. for 16 h before it was filtered through celite, and the filtrate was concentrated. The residue was diluted with water (100 mL) and extracted with EtOAc (2×500 mL). The combined organic layer was washed with brine, dried over Na₂SO₄, and concentrated under reduced pressure. The residue was purified by Combi-flash (5% MeOH in DCM) to afford methyl 2-fluoro-4-(1-methyl-1H-1,2,3-triazol-4-yl)benzoate (8.2 g, 81%) as a solid.

### Step 2:

To a solution of methyl 4-(1-methyl-1H-1,2,3-triazol-4-yl)benzoate (8.2 g, 34.86 mmol) in THF (40 mL), MeOH (20 mL) and water (20 mL) at room temperature was added lithium hydroxide mono-hydrate (4.3 g, 104.58 mmol). The reaction was stirred for 3 h before it was concentrated under reduced pressure. The residue was dissolved in water (20 mL) and extracted with EtOAc (50 mL). The aqueous layer was acidified with 1N aq. HCl (100 mL). The precipitate was collected by filtration and dried to afford 2-fluoro-4-(1-methyl-1H-1,2,3-triazol-4-yl)benzoic acid (6 g, 77%) as an off-white solid. LC-MS: [M+H⁺]=222.05.

### Step 3:

To a stirred solution of 2-fluoro-4-(1-methyl-1H-1,2,3-triazol-4-yl)benzoic acid (1 g, 4.52 mmol) in SOCl₂ (20 mL, 274 mmol) was added a catalytic amount of DMF (0.2 mL) at room temperature and the reaction mixture was stirred at 80° C. for 16 h. The reaction mixture was concentrated under reduced pressure to afford crude 2-fluoro-4-(1-methyl-1H-1,2,3-triazol-4-yl)benzoyl chloride (850 mg).

### Step 4:

To a stirred solution of 4-fluoro-2-methylbenzaldehyde (10 g, 72.4 mmol) in EtOH (50 mL) was added 2,2-dimethoxyethan-1-amine (7.61 g, 72.4 mmol) at room temperature followed by a catalytic amount of AcOH (0.1 mL) and the reaction mixture was stirred for 16 h. The reaction was cooled to 0° C. and NaBH₄ (4.11 g, 109 mmol) was added to reaction mixture and was stirred at room temperature for 4 h. The reaction mixture was diluted with water (50 mL) and extracted with EtOAc (2×10 mL). The combined organic layer was washed with brine, dried over Na₂SO₄, and concentrated. The residue was purified by Combi-flash (50-70% of EtOAc in hexane) to afford N-(4-fluoro-2-methylbenzyl)-2,2-dimethoxyethan-1-amine (14 g, 72%) as a liquid. LC-MS: [M+H⁺]=228.1.

### Step 5:

To a solution of N-(4-fluoro-2-methylbenzyl)-2,2-dimethoxyethan-1-amine (4 g, 17.60 mmol) in DCM (40 mL) was added 4-methylbenzenesulfonyl chloride (6.71 g, 35.2 mmol) at room temperature and stirred for 3 h. The reaction mixture was diluted with water (100 mL) and extracted with EtOAc (2×100 mL). The combined organic layer was washed with brine, dried over Na₂SO₄, and concentrated. The residue was purified by Combi-flash (15-20% EtOAc in hexane) to afford N-(2,2-dimethoxyethyl)-N-(4-fluoro-2-methylbenzyl)-4-methylbenzenesulfonamide (6 g, 89%) as an off-white solid.

### Step 6:

A solution of AlCl₃ (10.49 g, 79 mmol) in DCM (50 mL) was cooled to 0° C. and stirred for 30 min and followed by dropwise addition of N-(2,2-dimethoxyethyl)-N-(4-fluoro-2-methylbenzyl)-4-methylbenzenesulfonamide (6 g, 15.73 mmol) in DCM (20 mL) at room temperature. The reaction mixture was stirred for 3.5 h before it was poured into ice water mixture (200 mL) and extracted with EtOAc (2×100 mL). The combined organic layer was washed with brine, dried over Na₂SO₄, and concentrated. The residue was purified by Combi-flash (40-50% of EtOAc in hexane) to afford 6-fluoro-8-methylisoquinoline (1.3 g, 39%) as a light brown solid. LC-MS: [M+H⁺]=162.1.

### Step 7:

To a solution of 6-fluoro-8-methylisoquinoline (1.3 g, 8.07 mmol) in DCM (15 mL) at room temperature was added m-CPBA (4.18 g, 24.20 mmol). The reaction was stirred for 1.5 h before it was diluted with NaHCO₃ solution (50 mL) and extracted with 10% MeOH in DCM (2×100 mL). The combined organic layer was washed with brine, dried over Na₂SO₄, and concentrated. The residue was purified by Combi-flash (10% MeOH in DCM) to afford 6-fluoro-8-methylisoquinoline 2-oxide (800 mg, 41%) as a brown solid. LC-MS: [M+H⁺]=178.1.

### Step 8:

To a solution of 6-fluoro-8-methylisoquinoline 2-oxide (800 mg, 4.52 mmol) in DCM (4 mL) at room temperature was added tert-butyl (R)-3-aminopiperidine-1-carboxylate (904 mg, 4.52 mmol), DIPEA (3.15 mL, 18.06 mmol) and PyBroP (2.53 g, 5.42 mmol). The reaction mixture was stirred for 16 h before it was diluted with water (50 mL) and extracted with EtOAc (2×100 mL). The combined organic layer was washed with brine solution, dried over Na₂SO₄, and concentrated. The residue was purified by Combi-flash (50-60% of EtOAc in hexane) to afford tert-butyl (R)-3-((6-fluoro-8-methylisoquinolin-1-yl)amino)piperidine-1-carboxylate (500 mg, 30%) as a off-white solid. LC-MS: [M+H⁺]=360.2.

### Step 9:

To a solution of tert-butyl (R)-3-((6-fluoro-8-methylisoquinolin-1-yl)amino)-piperidine-1-carboxylate (150 mg, 0.417 mmol) in THF (5 mL) at 0° C. was added lithium bis(trimethylsilyl)amide (0.894 mL, 1.252 mmol) and the reaction mixture was stirred for 15 min and followed by addition of 2-fluoro-4-(1-methyl-1H-1,2,3-triazol-4-yl)benzoylchloride (300 mg, 1.252 mmol) in THF (5 mL). The reaction was stirred at 25° C. for 15 min before it was quenched with NH₄Cl solution and extracted with EtOAc (2×100 mL). The combined organic layer was washed with brine (50 mL), dried over Na₂SO₄, and concentrated. The residue was purified by Combi-flash (60-70% EtOAc in hexane) first and then was further purified by Prep-HPLC (Conditions: Column: LUNA (250×21.2×5 μm), Mobile phase A: 0.1% formic acid in water (100%), Mobile phase B: 100% MeCN, Gradient: Time (min)/% B: 0/55, 10/80, Flow: 15 mL/min, Dilution: MeCN+water). The pure fractions were combined and concentrated under reduced pressure to afford tert-butyl (R)-3-(2-fluoro-N-(6-fluoro-8-methylisoquinolin-1-yl)-4-(1-methyl-1H-1,2,3-triazol-4-yl)benzamido)piperidine-1-carboxylate (30 mg, 49%) as pale-yellow solid. LC-MS: [M+H⁺]=563.35.

### Step 10:

To a solution of tert-butyl (R)-3-(2-fluoro-N-(6-fluoro-8-methylisoquinolin-1-yl)-4-(1-methyl-1H-1,2,3-triazol-4-yl)benzamido)piperidine-1-carboxylate (95 mg, 0.169 mmol) in DCM (4 mL) at 0° C. was added 4M HCl in 1,4-dioxane (3 mL, 0.169 mmol). The reaction was stirred at room temperature for 1 h. The reaction mixture was concentrated and dried by lyophilization to afford (R)-2-fluoro-N-(6-fluoro-8-methylisoquinolin-1-yl)-4-(1-methyl-1H-1,2,3-triazol-4-yl)-N-(piperidin-3-yl)benzamide hydrochloride salt (80 mg, 93%) as an off-white solid. LC-MS: [M+H⁺]=463.3. ¹H NMR (CD₃OD, 400 MHZ): δ 8.43-8.39 (m, 1H), 8.21 (s, 1H), 7.71-7.67 (m, 1H), 7.34-7.25 (m, 3H), 7.18-7.15 (m, 1H), 6.89-6.84 (m, 1H), 5.09-5.03 (m, 1H), 4.11 (s, 3H), 3.99-3.97 (m, 1H), 3.85-3.82 (m, 1H), 3.42-3.39 (m, 1H), 3.04-2.99 (m, 1H), 2.83 (s, 3H), 2.70-2.68 (m, 1H), 1.97-1.90 (m, 2H), 1.65-1.58 (m, 1H).

### Synthesis of N-(2-bromothieno[3, 2-c]pyridin-4-yl)-3-hydroxy-4-(1-methylpyrazol-4-yl)-N-[(3R)-3-piperidyl] benzamide (Example 475)

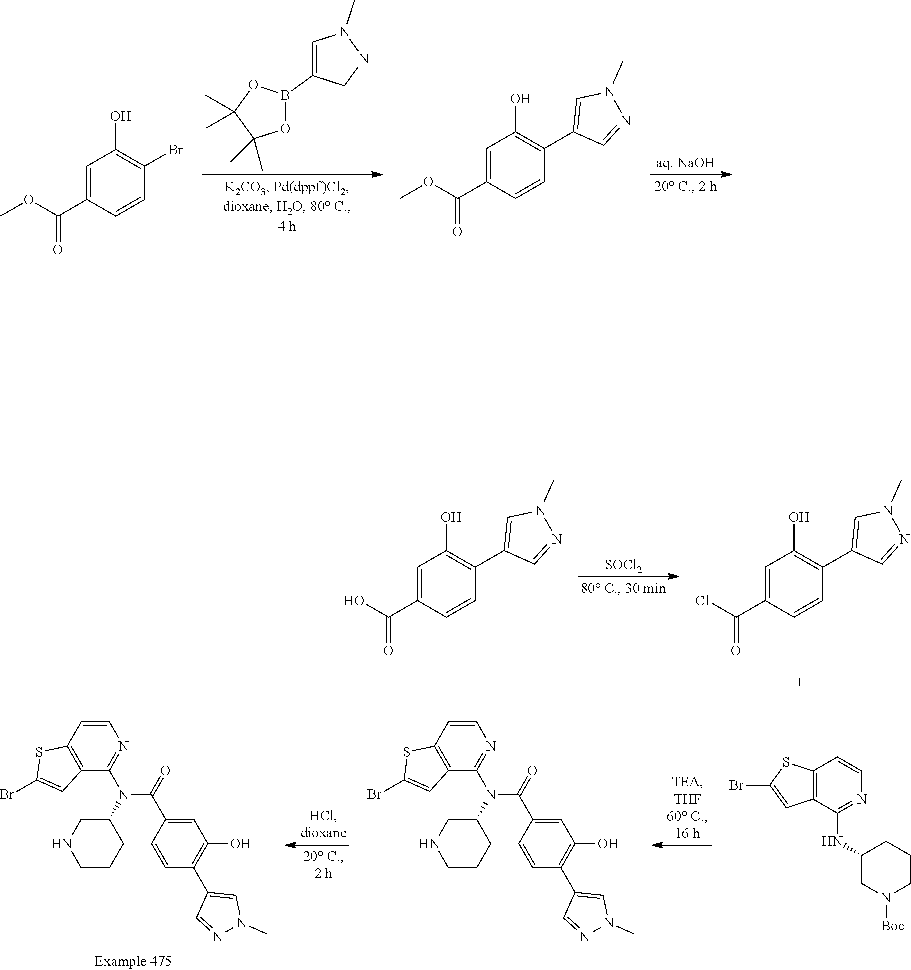

### Step 1:

To a solution of 1-methyl-4-(4,4,5,5-tetramethyl-1,3,2-dioxaborolan-2-yl) pyrazole (54.04 g, 259.7 mmol) and methyl 4-bromo-3-hydroxy-benzoate (20 g, 86.56 mmol) in dioxane (200 mL) and H₂O (80 mL) was added Pd(dppf)Cl₂ (6.34 g, 8.66 mmol) and K₂CO₃ (35.9 g, 259.7 mmol) at 20° C. under N₂. The mixture was heated to 80° C. and stirred for 4 h. The reaction mixture was filtered and concentrated under reduced pressure to give a residue. The residue was purified by column chromatography (SiO₂, Petroleum ether/Ethyl acetate=2/1 to 1/1) to give methyl 3-hydroxy-4-(1-methylpyrazol-4-yl) benzoate (14 g, 69%) as a white solid.

### Step 2:

To a solution of methyl 3-hydroxy-4-(1-methylpyrazol-4-yl) benzoate (6 g, 25.84 mmol) in tetrahydrofuran (60 mL), ethyl alcohol (20 mL) and water (20 mL) was added NaOH (5.17 g, 129.18 mmol). The mixture was stirred at 20° C. for 2 h. The reaction mixture was concentrated under reduced pressure to give a residue. The mixture was acidified by adding 1 N hydrochloric acid dropwise degree to pH=2. Solid was precipitated out. The mixture was filtered to give the filter cake. The filter cake was collected and dried to give crude 3-hydroxy-4-(1-methylpyrazol-4-yl) benzoic acid (3.6 g, 63%) as a white solid.

### Step 3:

A solution of crude 3-hydroxy-4-(1-methylpyrazol-4-yl) benzoic acid (860 mg, 3.94 mmol,) in SOCl₂ (3 mL) was degassed and purged with N₂ for 3 times at 0° C., and then the mixture was heated to 80° C. and stirred 2 h under N₂ atmosphere. The reaction mixture was concentrated to give crude 3-hydroxy-4-(1-methylpyrazol-4-yl)benzoyl chloride (860 mg, 92%).

### Step 4:

To a solution of tert-butyl (3R)-3-[(2-bromothieno [3, 2-c] pyridin-4-yl) amino] piperidine-1-carboxylate (500 mg, 1.21 mmol) and TEA (613.50 mg, 6.06 mmol) in tetrahydrofuran (2 mL) at 0° C. under N₂ atmosphere was added 3-hydroxy-4-(1-methylpyrazol-4-yl)benzoyl chloride (860.89 mg, 3.64 mmol). The mixture was heated to 60° C. and stirred for 16 h. The reaction mixture was concentrated. The residue was purified by Prep-TLC (SiO₂, EtOAc:Petroleum ether=3:1) to give tert-butyl (3R)-3-[(2-bromothieno[3, 2-c] pyridin-4-yl)-[3-hydroxy-4-(1-methylpyrazol-4-yl) benzoyl] amino] piperidine-1-carboxylate (400 mg, 53%) as a white solid.

### Step 5:

The title compound hydrochloride salt was obtained as an off-white solid by deprotection Method A used in Example 97. LC-MS: [M+H⁺]=514.2. ¹H NMR (400 MHZ, CD₃OD): δ 8.48 (s, 1H), 8.45-8.38 (m, 2H), 7.88 (br d, J=5.3 Hz, 1H), 7.51 (s, 1H), 7.32 (br d, J=8.0 Hz, 1H), 6.90 (s, 1H), 6.73 (br d, J=7.6 Hz, 1H), 5.27-4.97 (m, 1H), 4.07 (s, 3H), 3.79 (br d, J=10.3 Hz, 1H), 3.65-3.54 (m, 1H), 3.35 (br s, 1H), 2.86 (br t, J=12.3 Hz, 1H), 2.22-1.81 (m, 3H), 1.56-1.24 (m, 1H).

### Synthesis of 3-hydroxy-N-[2-(5-hydroxypent-1-ynyl) thieno[3, 2-c] pyridin-4-yl]-4-(1-methylpyrazol-4-yl)-N-[(3R)-3-piperidyl] benzamide (Example 476)

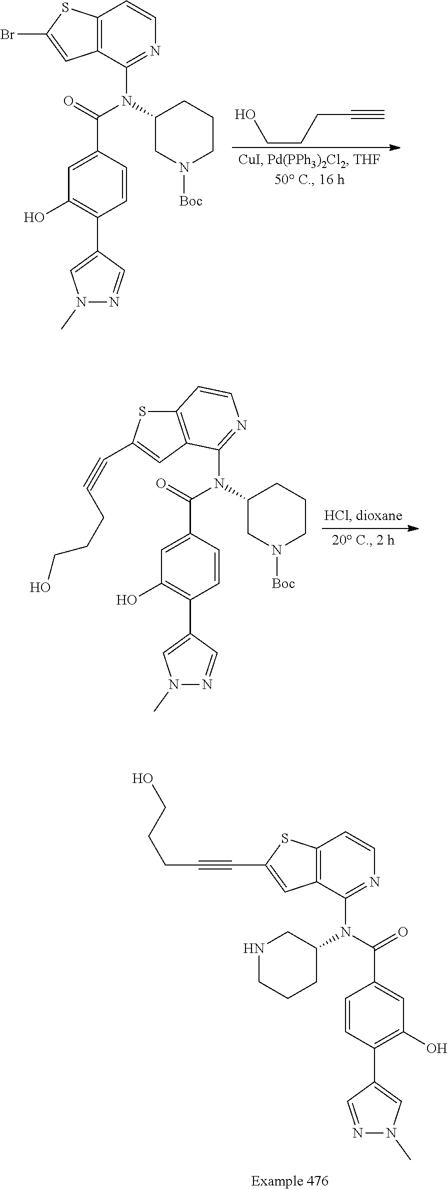

### Step 1:

To a solution of tert-butyl (3R)-3-[(2-bromothieno[3, 2-c]pyridin-4-yl)-[3-hydroxy-4-(1-methylpyrazol-4-yl) benzoyl] amino] piperidine-1-carboxylate (400 mg, 653.02 μmol) and pent-4-yn-1-ol (549.30 mg, 6.53 mmol) in tetrahydrofuran (6 mL) and TEA (2 mL) was added CuI (12.44 mg, 65.30 μmol) and Pd(PPh₃)₂Cl₂ (45.84 mg, 65.30 μmol) at 20° C. under N₂. The mixture was heated to 50° C. and stirred for 16 h under N₂. The reaction mixture was filtered and concentrated under reduced pressure. The residue was purified by Prep-HPLC (column: Waters Xbridge Prep OBD C18 150*40 mm*10 μm; mobile phase: [H₂O (10 mM NH₄HCO₃)-MeCN]; gradient: 35%-65% B over 8.0 min) to give tert-butyl (3R)-3-[[3-hydroxy-4-(1-methylpyrazol-4-yl)benzoyl]-[2-(5-hydroxypent-1-ynyl)thieno[3, 2-c]pyridin-4-yl]amino]piperidine-1-carboxylate (190 mg, 47%) as a white solid.

### Step 2:

The title compound hydrochloride salt was obtained as an off-white solid by deprotection Method A used in Example 97. LC-MS: [M+H⁺]=516.4. ¹H NMR (400 MHZ, CD₃OD): δ 8.39 (d, J=5.5 Hz, 1H), 8.21 (s, 1H), 8.08 (d, J=1.6 Hz, 1H), 7.82 (d, J=5.5 Hz, 1H), 7.32 (s, 1H), 7.25 (br d, J=8.1 Hz, 1H), 6.88 (s, 1H), 6.69 (br d, J=7.6 Hz, 1H), 5.27-4.93 (m, 1H), 3.95 (s, 3H), 3.79-3.71 (m, 1H), 3.68 (t, J=6.2 Hz, 2H), 3.34 (br s, 1H), 2.93-2.77 (m, 1H), 2.58 (t, J=7.1 Hz, 2H), 2.13-1.75 (m, 6H), 1.55-1.25 (m, 1H).

### Synthesis of N-(2-cyanothieno [3, 2-c] pyridin-4-yl)-2-fluoro-4-(1-methyltriazol-4-yl)-N-[(3R)-3-piperidyl] benzamide (Example 477)

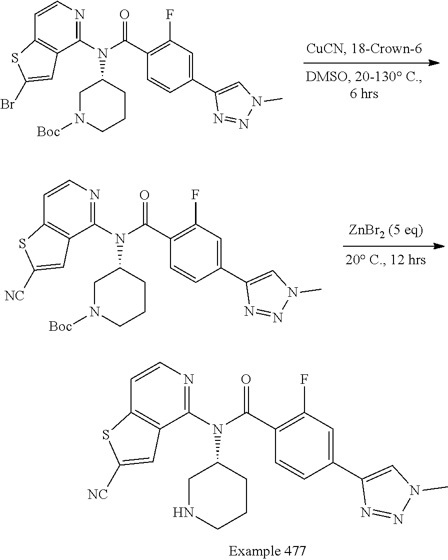 [SMILES: Cn1cc(-c2ccc(C(=O)N(c3nccc4sc(Br)cc34)[C@@H]3CCCN(C(=O)OC(C)(C)C)C3)c(F)c2)nn1 ; Cn1cc(-c2ccc(C(=O)N(c3nccc4sc(C#N)cc34)[C@@H]3CCCN(C(=O)OC(C)(C)C)C3)c(F)c2)nn1 ; Cn1cc(-c2ccc(C(=O)N(c3nccc4sc(C#N)cc34)[C@@H]3CCCNC3)c(F)c2)nn1]

### Step 1:

To a solution of tert-butyl (3R)-3-[(2-bromothieno[3,2-c]pyridin-4-yl)-[2-fluoro-4-(1-methyltriazol-4-yl)benzoyl]amino]piperidine-1-carboxylate (100 mg, 162.47 μmol) in DMSO (2 mL) at 20° C. was added CuCN (72.75 mg, 812.33 μmol) and 18-CROWN-6 (128.83 mg, 487.40 μmol), then the mixture was stirred at 130° C. for 6 h under N₂ atmosphere. The mixture was poured into water (10 mL) and extracted with ethyl acetate (3×10 mL). The combined organic phase was washed with brine (10 mL), dried with anhydrous sodium sulfate, and concentrated under reduced pressure to give tert-butyl (3R)-3-[(2-cyanothieno[3,2-c]pyridin-4-yl)-[2-fluoro-4-(1-methyltriazol-4-yl)benzoyl]amino]piperidine-1-carboxylate (50 mg, 54%) as a yellow solid.

### Step 2:

The title compound hydrochloride salt was obtained as a white solid by deprotection Method A used in Example 97. LC-MS: [M+H]=462.3. ¹H NMR (400 MHZ, DMSO-d₆): δ 9.12-8.73 (m, 2H), 8.70-8.59 (m, 1H), 8.58-8.41 (m, 2H), 8.26-8.08 (m, 1H), 8.06-7.74 (m, 1H), 7.53-7.31 (m, 2H), 7.29-7.04 (m, 1H), 5.18 (br d, J=9.4 Hz, 1H), 4.10-3.97 (m, 3H), 3.77-3.62 (m, 1H), 3.21-3.15 (m, 1H), 2.69-2.61 (m, 1H), 2.02-1.64 (m, 4H), 1.28-1.03 (m, 1H).

### Synthesis of (R)—N-(2-(2-aminopyridin-4-yl)thieno[3,2-c]pyridin-4-yl)-2-fluoro-4-(1-methyl-1H-1,2,3-triazol-4-yl)-N-(piperidin-3-yl)benzamide (Example 478)

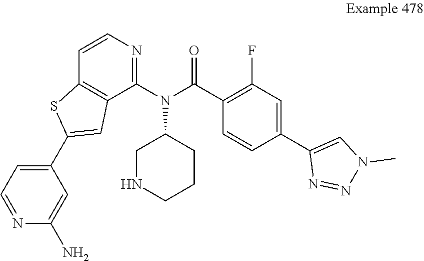 [SMILES: Cn1cc(-c2ccc(C(=O)N(c3nccc4sc(-c5ccnc(N)c5)cc34)[C@@H]3CCCNC3)c(F)c2)nn1]

Example 478 was made following the synthesis of example 363 by substituting the 4-pyridylboronic acid for (2-amino-4-pyridyl)boronic acid, provided the title compound hydrochloride salt as a white solid. LC-MS: [M+H]=529.2. ¹H NMR (400 MHZ, DMSO-d₆): δ 9.13-8.84 (m, 2H), 8.44 (br d, J=11.5 Hz, 2H), 8.17-8.08 (m, 3H), 7.58-7.48 (m, 1H), 7.38-7.16 (m, 4H), 5.23-4.87 (m, 1H), 4.01 (s, 3H), 3.76-3.68 (m, 1H), 3.24-3.14 (m, 1H), 2.79-2.67 (m, 2H), 2.07-1.69 (m, 3H), 1.17-1.05 (m, 1H).

### Synthesis of (R)-2-fluoro-N-(2-(3-hydroxyprop-1-yn-1-yl)thieno[3,2-c]pyridin-4-yl)-4-(1-methyl-1H-1,2,3-triazol-4-yl)-N-(piperidin-3-yl)benzamide (Example 479)

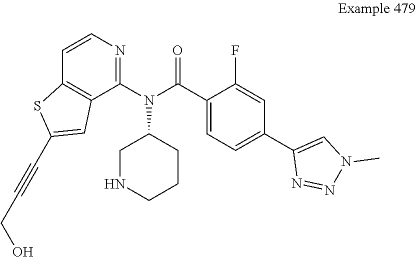 [SMILES: Cn1cc(-c2ccc(C(=O)N(c3nccc4sc(C#CCO)cc34)[C@@H]3CCCNC3)c(F)c2)nn1]

Example 479 was prepared according to the procedure described for Example 376 by substituting 2-methylbut-3-yn-2-ol with tert-butyl-dimethyl-prop-2-ynoxy-silane, provided the title compound hydrochloride salt as a white solid. LC-MS: [M+H⁺]=491.2. ¹H NMR (400 MHZ, DMSO-d₆): δ 8.96-8.69 (m, 2H), 8.49-8.36 (m, 2H), 8.02-7.71 (m, 2H), 7.46-7.30 (m, 2H), 7.23-7.08 (m, 1H), 5.59-5.46 (m, 1H), 5.07-4.80 (m, 1H), 4.47-4.32 (m, 2H), 4.02 (br s, 3H), 3.69 (td, J=2.3, 7.6 Hz, 1H), 3.20-3.09 (m, 1H), 1.78 (br s, 3H), 1.26-0.99 (m, 1H).

### Synthesis of N-[2-(3-carbamoylphenyl) thieno [3, 2-c] pyridin-4-yl]-2-fluoro-4-(1-methyltriazol-4-yl)-N-[(3R)-3-piperidyl] benzamide (Example 480)

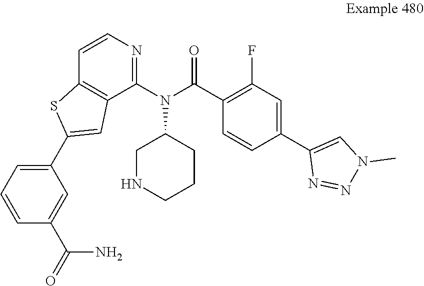 [SMILES: Cn1cc(-c2ccc(C(=O)N(c3nccc4sc(-c5cccc(C(N)=O)c5)cc34)[C@@H]3CCCNC3)c(F)c2)nn1]

Example 481 was prepared according to the procedure described for Example 363 by substituting 4-pyridylboronic acid with (3-carbamoylphenyl)boronic acid, provided the title compound hydrochloride salt as a white solid. LC-MS: [M+H⁺]=556.3. ¹H NMR (400 MHz, DMSO-d₆): δ 9.25 (br d, J=9.8 Hz, 2H), 8.46-8.39 (m, 1H), 8.39-8.28 (m, 2H), 8.27-8.17 (m, 1H), 8.11 (s, 1H), 8.07-7.90 (m, 3H), 7.69-7.52 (m, 2H), 7.43-7.17 (m, 3H), 5.24-4.88 (m, 1H), 3.99 (s, 3H), 3.77-3.71 (m, 1H), 3.42-3.28 (m, 1H), 3.18 (br d, J=10.3 Hz, 1H), 2.85-2.70 (m, 1H), 2.29-2.02 (m, 1H), 1.95-1.73 (m, 2H), 1.31-1.02 (m, 1H).

### Synthesis of (R)-2-fluoro-N-(2-(5-(hydroxymethyl)thiophen-2-yl)thieno[3,2-c]pyridin-4-yl)-4-(1-methyl-1H-1,2,3-triazol-4-yl)-N-(piperidin-3-yl)benzamide (Example 481)

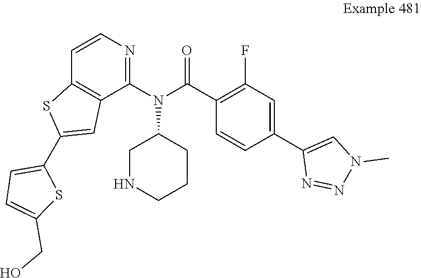 [SMILES: Cn1cc(-c2ccc(C(=O)N(c3nccc4sc(-c5ccc(CO)s5)cc34)[C@@H]3CCCNC3)c(F)c2)nn1]

Example 481 was prepared according to the procedure described for Example 363 by substituting 4-pyridylboronic acid with [5-(hydroxymethyl)-2-thienyl]boronic acid, provided the title compound hydrochloride salt (88%) as a yellow solid. LC-MS: [M+H⁺]=549.3. ¹H NMR (400 MHZ, CD₃OD): δ 8.34 (br d, J=5.5 Hz, 1H), 8.20 (s, 1H), 7.83 (br d, J=5.3 Hz, 1H), 7.51-7.43 (m, 1H), 7.37 (br d, J=10.6 Hz, 1H), 7.32 (br d, J=6.5 Hz, 2H), 7.22-7.12 (m, 1H), 7.02 (d, J=3.4 Hz, 1H), 5.29-5.19 (m, 1H), 4.81 (s, 2H), 4.08 (s, 3H), 3.89 (br d, J=10.6 Hz, 1H), 3.73-3.63 (m, 1H), 3.38 (br s, 2H), 2.95-2.84 (m, 1H), 2.16-1.82 (m, 3H), 1.46-1.29 (m, 1H).

### Synthesis of 2-fluoro-N-[(3R)-3-piperidyl]-N-(2-pyrazin-2-ylthieno[3,2-c]pyridin-4-yl)-4-(triazolo[4,5-b]pyridin-3-yl)benzamide (Example 482)

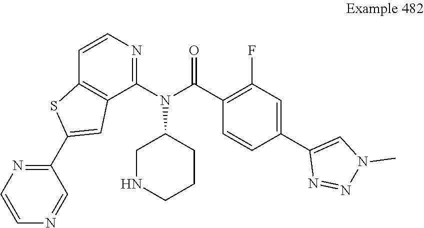 [SMILES: Cn1cc(-c2ccc(C(=O)N(c3nccc4sc(-c5cnccn5)cc34)[C@@H]3CCCNC3)c(F)c2)nn1]

Example 482 was prepared according to the procedure described for Example 363 by substituting 4-pyridylboronic acid with pyrazin-2-ylboronic acid, provided the title compound hydrochloride salt as a white solid. LC-MS: [M+H⁺]=515.3. ¹H NMR (400 MHZ, CD₃OD): δ 9.38 (br s, 1H), 8.63 (br d, J=16.1 Hz, 2H), 8.40 (br d, J=5.1 Hz, 1H), 8.32-8.11 (m, 2H), 7.93 (br s, 1H), 7.44-7.14 (m, 3H), 5.26 (br s, 1H), 4.06 (s, 3H), 3.91 (br d, J=9.5 Hz, 1H), 3.77-3.58 (m, 2H), 3.37 (br s, 1H), 3.01-2.77 (m, 1H), 2.22-1.80 (m, 2H), 1.55-1.34 (m, 1H).

### Synthesis of I (R)-2-fluoro-4-(1-methyl-1H-1,2,3-triazol-4-yl)-N-(2-(2-methylthiazol-5-yl)thieno[3,2-c]pyridin-4-yl)-N-(piperidin-3-yl)benzamide (Example 483)

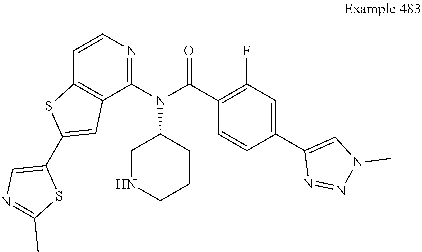 [SMILES: Cc1ncc(-c2cc3c(N(C(=O)c4ccc(-c5cn(C)nn5)cc4F)[C@@H]4CCCNC4)nccc3s2)s1]

Example 483 was prepared according to the procedure described for Example 363 by substituting 4-pyridylboronic acid with 2-methyl-5-(4,4,5,5-tetramethyl-1,3,2-dioxaborolan-2-yl)thiazole, provided the title compound hydrochloride salt as an off-white solid. LC-MS: [M+H⁺]=534.3. ¹H NMR (400 MHZ, CD₃OD): δ 8.43-8.32 (m, 1H), 8.28-8.15 (m, 2H), 7.92-7.78 (m, 1H), 7.72-7.59 (m, 1H), 7.41-7.12 (m, 3H), 5.22 (br dd, J=8.9, 9.6 Hz, 1H), 4.12-4.04 (m, 3H), 3.88 (br d, J=12.0 Hz, 1H), 3.75-3.61 (m, 1H), 3.31 (br s, 2H), 2.89 (s, 3H), 2.11-1.81 (m, 3H), 1.47-1.26 (m, 1H).

### Synthesis of N-[2-(2-aminopyrimidin-4-yl)thieno[3,2-c]pyridin-4-yl]-2-fluoro-4-(1-methyltriazol-4-yl)-N-[(3R)-3-piperidyl]benzamide (Example 484)

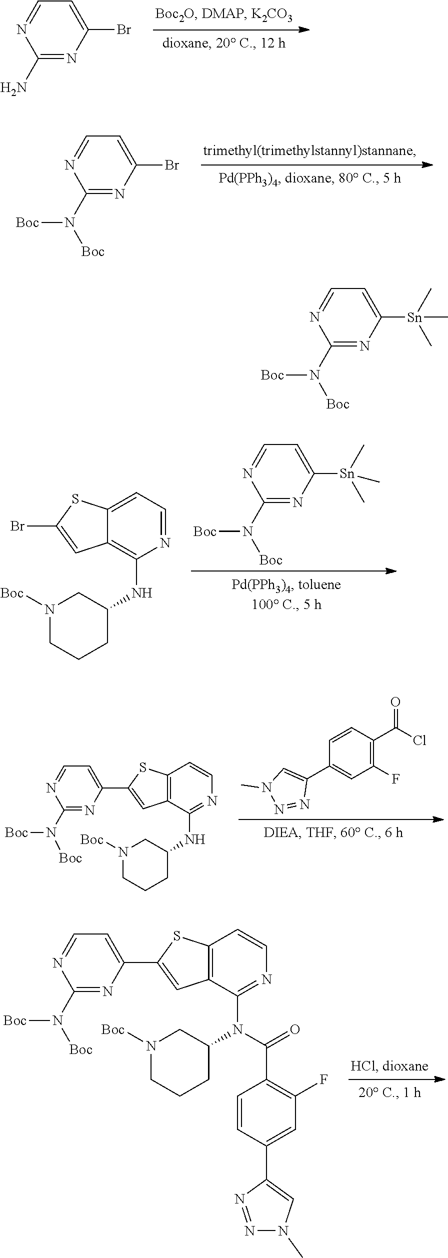

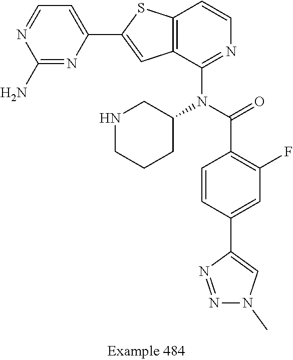 [SMILES: Cn1cc(-c2ccc(C(=O)N(c3nccc4sc(-c5ccnc(N)n5)cc34)[C@@H]3CCCNC3)c(F)c2)nn1]

### Step 1:

A mixture of 4-bromopyrimidin-2-amine (2.87 g, 16.49 mmol), Boc₂O (11.52 g, 52.78 mmol), DMAP (201.51 mg, 1.65 mmol), and K₂CO₃ (13.68 g, 98.97 mmol) in dioxane (30 mL) at 20° C. was degassed and purged with N₂, and then the mixture was stirred at 20° C. for 12 h. The reaction mixture was poured into water (100 mL), and extracted with EtOAc (3×50 mL). The combined organic layer was washed with brine (50 mL×2), dried over Na₂SO₄, filtered, and concentrated. The residue was triturated with petroleum ether (100 mL) and ethyl acetate (10 mL) for 30 min. The precipitate was collected and dried to give tert-butyl N-(4-bromopyrimidin-2-yl)-N-tert-butoxycarbonyl-carbamate (2 g, 32%) as a white solid.

### Step 2:

A mixture of tert-butyl N-(4-bromopyrimidin-2-yl)-N-tert-butoxycarbonyl-carbamate (2 g, 5.34 mmol), trimethyl(trimethylstannyl)stannane (3.55 g, 10.84 mmol) and palladium triphenylphosphane (197.04 mg, 534.43 μmol) in dioxane (20 mL) was degassed and purged with N₂ 3 times at 20° C., and then the mixture was heated to 80° C. and stirred for 5 h under N₂. The reaction mixture was cooled to 25° C. and extracted with ethyl acetate (3×100 mL). The combined organic layer was washed with brine (2×100 mL), dried over Na₂SO₄, filtered, and concentrated. The residue was purified by column chromatography (SiO₂, Petroleum ether/Ethyl acetate=0/1 to 4/1) to give tert-butyl N-tert-butoxycarbonyl-N-(4-trimethylstannylpyrimidin-2-yl)carbamate (1.8 g, 73%) as a yellow solid.

### Step 3:

A mixture of tert-butyl N-tert-butoxycarbonyl-N-(4-trimethylstannylpyrimidin-2-yl)carbamate (1.33 g, 2.91 mmol), tert-butyl (3R)-3-[(2-bromothieno[3,2-c]pyridin-4-yl)amino]piperidine-1-carboxylate (400 mg, 970.06 μmol), and palladium triphenylphosphane (107.30 mg, 291.02 μmol) in toluene (10 mL) at 20° C. was degassed and purged with N₂, and then the mixture was heated to 100° C. and stirred for 3 h. The reaction mixture was concentrated under reduced pressure. The residue was purified by column chromatography (SiO₂, Petroleum ether/Ethyl acetate=1/1) to give tert-butyl (3R)-3-[[2-[2-[bis(tert-butoxycarbonyl)amino]pyrimidin-4-yl]thieno[3,2-c]pyridin-4-yl]amino]piperidine-1-carboxylate (500 mg, 82%) as a yellow solid.

### Step 4:

A mixture of tert-butyl (3R)-3-[[2-[2-[bis(tert-butoxycarbonyl)amino]pyrimidin-4-yl]thieno[3,2-c]pyridin-4-yl]amino]piperidine-1-carboxylate (300 mg, 478.65 μmol), 2-fluoro-4-(1-methyltriazol-4-yl)benzoylchloride (573.50 mg, 2.39 mmol), and DIEA (185.58 mg, 1.44 mmol) in THF (4 mL) at 20° C. was degassed and purged with N₂, and then the mixture was heated to 60° C. and stirred for 12 h. The mixture was cooled to room temperature, filtered, and the filtrate was concentrated. The residue was purified by Prep-HPLC (column: Phenomenex Luna C18 100*30 mm*3 μm; mobile phase: [H₂O (0.04% HCl)-MeCN]; gradient: 70%-95% B over 8.0 min) to give tert-butyl (3R)-3-[[2-[2-[bis(tert-butoxycarbonyl)amino]pyrimidin-4-yl]thieno[3,2-c]pyridin-4-yl]-[2-fluoro-4-(1-methyltriazol-4-yl)benzoyl]amino]piperidine-1-carboxylate (45 mg, 11%) as a yellow solid.

### Step 5:

The title compound hydrochloride salt was obtained as a white solid by deprotection Method A used in Example 97. LC-MS: [M+H⁺]=530.4. ¹H NMR (400 MHZ, CD₃OD): δ 8.59 (br s, 1H), 8.49 (d, J=5.6 Hz, 1H), 8.40 (d, J=6.6 Hz, 1H), 8.19 (br s, 1H), 7.96 (br d, J=5.3 Hz, 1H), 7.76 (br d, J=6.6 Hz, 1H), 7.36-7.14 (m, 3H), 5.38-5.16 (m, 1H), 4.07 (s, 3H), 3.97-3.79 (m, 1H), 3.78-3.69 (m, 1H), 3.42-3.33 (m, 1H), 2.97-2.82 (m, 1H), 2.15-1.83 (m, 3H), 1.52-1.20 (m, 2H).

### Synthesis of 2-fluoro-4-(1-methyltriazol-4-yl)-N-[(3R)-3-piperidyl]-N-[2-[1-(3-pyridylmethyl) pyrazol-4-yl] thieno [3, 2-c] pyridin-4-yl] benzamide (Example 485)

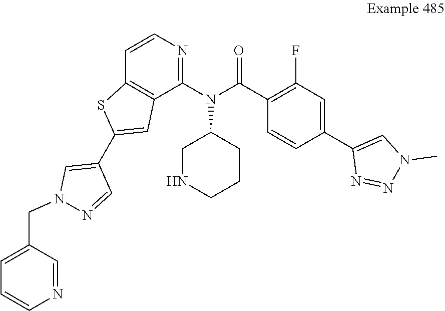 [SMILES: Cn1cc(-c2ccc(C(=O)N(c3nccc4sc(-c5cnn(Cc6cccnc6)c5)cc34)[C@@H]3CCCNC3)c(F)c2)nn1]

Example 485 was prepared according to the procedure described for Example 363 by substituting 4-pyridylboronic acid with 3-[[4-(4,4,5,5-tetramethyl-1, 3, 2-dioxaborolan-2-yl) pyrazol-1-yl] methyl] pyridine, provided the title compound hydrochloride salt as a white solid. LC-MS: [M+H⁺]=594.4. ¹H NMR (400 MHZ, CD₃OD): δ 8.95-8.87 (m, 2H), 8.63 (br d, J=7.9 Hz, 1H), 8.43 (br s, 1H), 8.34 (br s, 1H), 8.24 (br s, 1H), 8.16 (dd, J=5.9, 8.0 Hz, 1H), 8.07-7.86 (m, 2H), 7.61 (br s, 1H), 7.46-7.14 (m, 3H), 5.74 (s, 2H), 5.20 (br s, 1H), 4.09 (s, 3H), 3.90 (br d, J=10.4 Hz, 1H), 3.66 (br t, J=10.4 Hz, 1H), 3.35 (br d, J=12.6 Hz, 1H), 2.89 (br s, 1H), 2.18-1.81 (m, 3H), 1.45 (br d, J=3.9 Hz, 1H).

### Synthesis of N-[2-[(E)-3-amino-3-oxo-prop-1-enyl]thieno[3,2-c]pyridin-4-yl]-2-fluoro-4-(1-methyltriazol-4-yl)-N-[(3R)-3-piperidyl]benzamide (Example 486)

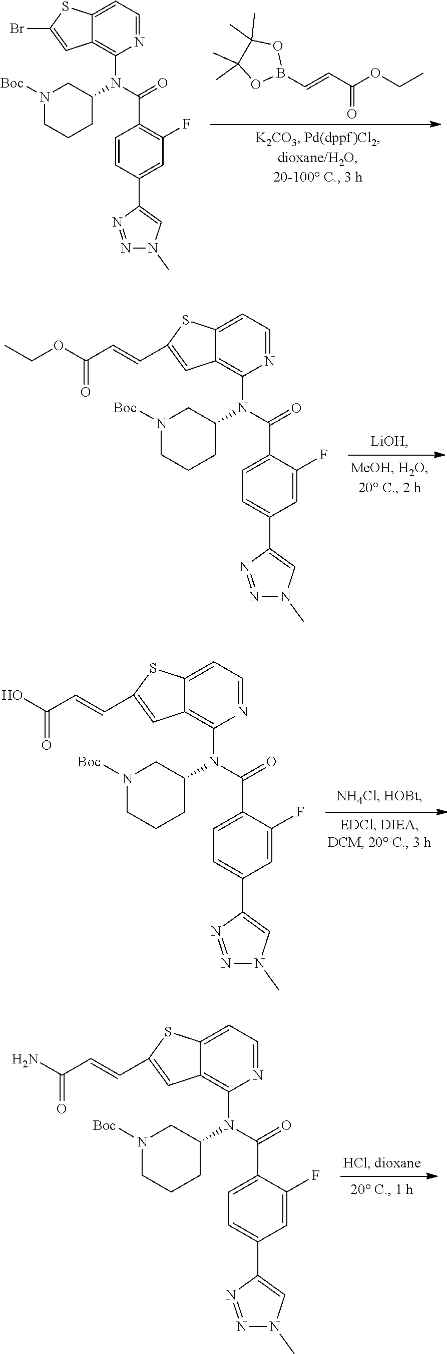 [SMILES: Cn1cc(-c2ccc(C(=O)N(c3nccc4sc(Br)cc34)[C@@H]3CCCN(C(=O)OC(C)(C)C)C3)c(F)c2)nn1 ; CCOC(=O)/C=C/B1OC(C)(C)C(C)(C)O1 ; CCOC(=O)/C=C/c1cc2c(N(C(=O)c3ccc(-c4cn(C)nn4)cc3F)[C@@H]3CCCN(C(=O)OC(C)(C)C)C3)nccc2s1 ; Cn1cc(-c2ccc(C(=O)N(c3nccc4sc(/C=C/C(=O)O)cc34)[C@@H]3CCCN(C(=O)OC(C)(C)C)C3)c(F)c2)nn1 ; Cn1cc(-c2ccc(C(=O)N(c3nccc4sc(/C=C/C(N)=O)cc34)[C@@H]3CCCN(C(=O)OC(C)(C)C)C3)c(F)c2)nn1]

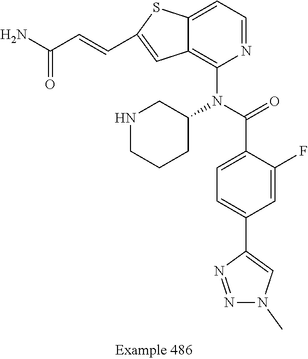 [SMILES: Cn1cc(-c2ccc(C(=O)N(c3nccc4sc(/C=C/C(N)=O)cc34)[C@@H]3CCCNC3)c(F)c2)nn1]

### Step 1:

To a solution of tert-butyl (3R)-3-[(2-bromothieno[3,2-c]pyridin-4-yl)-[2-fluoro-4-(1-methyltriazol-4-yl)benzoyl]amino]piperidine-1-carboxylate (0.2 g, 324.93 μmol) in dioxane (1.8 mL) and H₂O (0.2 mL) was added ethyl (E)-3-(4,4,5,5-tetramethyl-1,3,2-dioxaborolan-2-yl)prop-2-enoate (146.92 mg, 649.86 μmol), K₂CO₃ (134.73 mg, 974.79 μmol), and Pd(dppf)Cl₂ (23.78 mg, 32.49 μmol) at 20° C. The mixture was stirred at 100° C. for 3 hours under N₂. The reaction mixture was filtered, and the filter liquor was diluted with water (5 mL) and extracted with ethyl acetate (3×5 mL). The combined organic layer was washed with brine (6×5 mL), dried over Na₂SO₄, filtered, and concentrated. The residue was purified by column chromatography (SiO₂, Petroleum ether/Ethyl acetate=0/1) to give tert-butyl (3R)-3-[[2-[(E)-3-ethoxy-3-oxo-prop-1-enyl]thieno[3,2-c]pyridin-4-yl]-[2-fluoro-4-(1-methyltriazol-4-yl)benzoyl]amino]piperidine-1-carboxylate (0.13 g, 50%) as a yellow oil.

### Step 2:

To a solution of tert-butyl (3R)-3-[[2-[(E)-3-ethoxy-3-oxo-prop-1-enyl]thieno[3,2-c]pyridin-4-yl]-[2-fluoro-4-(1-methyltriazol-4-yl)benzoyl]amino]piperidine-1-carboxylate (0.13 g, 163.85 μmol) in MeOH (1.3 mL) and H₂O (0.65 mL) was added LiOH (19.62 mg, 819.26 μmol) at 20° C. Then the mixture was stirred at 20° C. for 2 h before it was acidified with 1N aq. HCl solution. The reaction mixture was concentrated under reduced pressure to give (E)-3-[4-[[(3R)-1-tert-butoxycarbonyl-3-piperidyl]-[2-fluoro-4-(1-methyltriazol-4-yl)benzoyl]amino]thieno[3,2-c]pyridin-2-yl]prop-2-enoic acid (0.13 g) as an orange oil, which was used directly in the next step.

### Step 3:

To a solution of (E)-3-[4-[[(3R)-1-tert-butoxycarbonyl-3-piperidyl]-[2-fluoro-4-(1-methyltriazol-4-yl)benzoyl]amino]thieno[3,2-c]pyridin-2-yl]prop-2-enoic acid (0.13 g, 214.29 μmol) in DCM (2.6 mL) was added HOBt (43.43 mg, 321.43 μmol) and EDCI (61.62 mg, 321.43 μmol) at 20° C., and the mixture was stirred at 20° C. for 5 minutes. Then DIEA (55.39 mg, 428.57 μmol, 74.65 μL) and NH₄Cl (34.39 mg, 642.86 μmol) were added dropwise to the reaction at 20° C. The mixture was stirred at 20° C. for 3 hours. The reaction mixture was diluted with water (5 mL) and extracted with dichloromethane (3×5 mL). The combined organic layer was washed with brine (6×5 mL), dried over Na₂SO₄, filtered, and concentrated under reduced pressure to give tert-butyl (3R)-3-[[2-[(E)-3-amino-3-oxo-prop-1-enyl]thieno[3,2-c]pyridin-4-yl]-[2-fluoro-4-(1-methyltriazol-4-yl)benzoyl]amino]piperidine-1-carboxylate (0.1 g, crude) as brown oil, which was used directly in the next step.

### Step 4:

The title compound hydrochloride salt was obtained as an off-white solid by deprotection Method A used in Example 97. LC-MS: [M+H⁺]=506.3. ¹H NMR (400 MHZ, DMSO-d₆): δ 9.23-8.80 (m, 2H), 8.53-8.30 (m, 2H), 7.98-7.66 (m, 4H), 7.44-7.14 (m, 3H), 6.50 (br d, J=15.4 Hz, 1H), 5.14-4.77 (m, 1H), 4.02 (s, 3H), 3.71-3.62 (m, 1H), 3.32-3.14 (m, 2H), 2.77-2.60 (m, 1H), 2.06-1.66 (m, 3H), 1.27-1.09 (m, 1H).

### Synthesis of N-(2-benzylthieno[3,2-c]pyridin-4-yl)-2-fluoro-4-(1-methyltriazol-4-yl)-N-[(3R)-3-piperidyl]benzamide (Example 487)

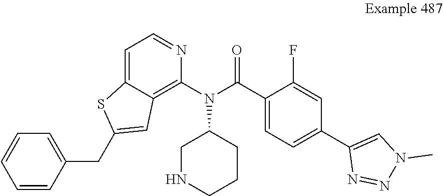 [SMILES: Cn1cc(-c2ccc(C(=O)N(c3nccc4sc(Cc5ccccc5)cc34)[C@@H]3CCCNC3)c(F)c2)nn1]

Example 487 was prepared according to the procedure described for Example 363 by substituting 4-pyridylboronic acid with 2-benzyl-4,4,5,5-tetramethyl-1,3,2-dioxaborolane, provided the title compound hydrochloride salt as a white solid. LC-MS: [M+H⁺]=527.3. ¹H NMR (400 MHZ, DMSO-d₆): δ 9.06-8.71 (m, 2H), 8.48 (s, 1H), 8.33-8.26 (m, 1H), 7.91-7.81 (m, 1H), 7.44-7.14 (m, 9H), 5.16-5.02 (m, 1H), 4.35-4.17 (m, 2H), 4.04 (s, 3H), 3.72-3.62 (m, 1H), 3.40-3.38 (m, 2H), 3.23-3.15 (m, 1H), 2.75-2.65 (m, 1H), 1.95-1.67 (m, 3H), 1.19-1.05 (m, 1H).

### Synthesis of 2-fluoro-4-(1-methyltriazol-4-yl)-N-[(3R)-3-piperidyl]-N-[2-(3-pyridylmethyl)thieno[3,2-c]pyridin-4-yl]benzamide (Example 488)

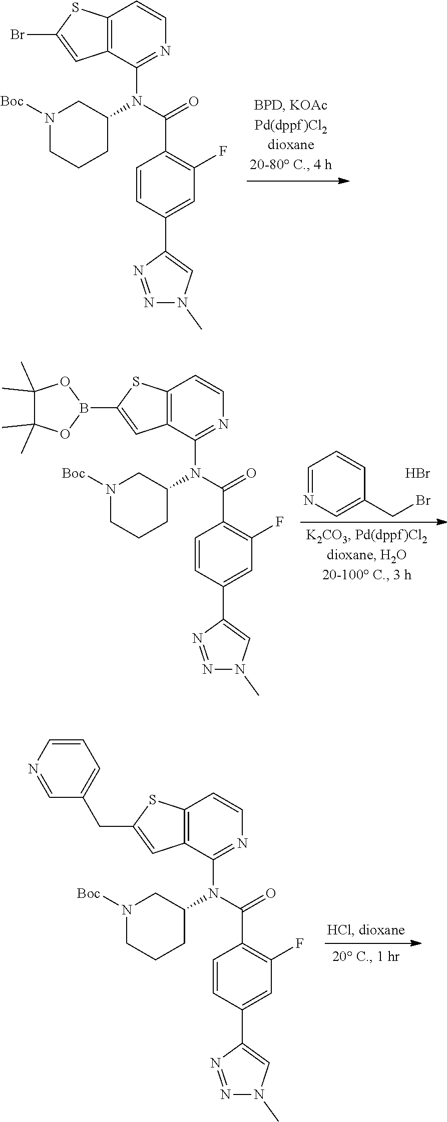

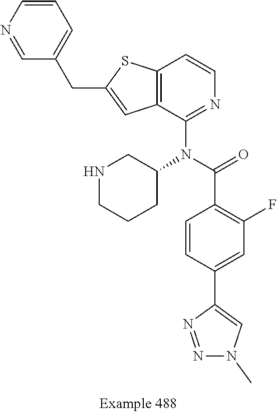 [SMILES: Cn1cc(-c2ccc(C(=O)N(c3nccc4sc(Cc5cccnc5)cc34)[C@@H]3CCCNC3)c(F)c2)nn1]

Example 488 was prepared according to the procedure described for Example 415 by substituting 4-bromo-1-isobutyl-triazole with 3-(bromomethyl)pyridine, provided the title compound hydrochloride salt as an off-white solid. LC-MS: [M+H⁺]=528.3. ¹H NMR (400 MHz, DMSO-d₆): δ 9.07-8.84 (m, 2H), 8.74-8.66 (m, 2H), 8.47 (s, 1H), 8.32 (br d, J=5.6 Hz, 1H), 7.98-7.87 (m, 2H), 7.71-7.62 (m, 1H), 7.38-7.25 (m, 3H), 7.15-7.04 (m, 1H), 5.13-5.01 (m, 1H), 4.46-4.36 (m, 2H), 4.04 (s, 3H), 3.67 (br d, J=7.4 Hz, 2H), 3.22-3.18 (m, 2H), 1.94-1.76 (m, 3H), 1.12-1.09 (m, 1H).

### Synthesis of N-[2-[1-(2-amino-2-oxo-ethyl)pyrazol-4-yl]thieno[3,2-c]pyridin-4-yl]-2-fluoro-4-(1-methyltriazol-4-yl)-N-[(3R)-3-piperidyl]benzamide (Example 489)

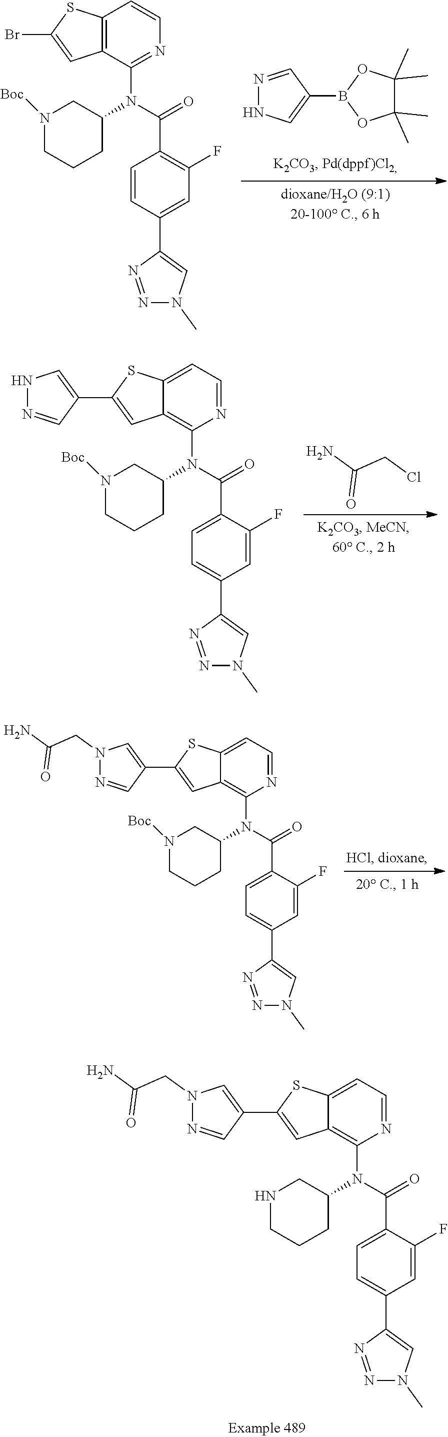

### Step 1:

A mixture of 4-(4,4,5,5-tetramethyl-1,3,2-dioxaborolan-2-yl)-1H-pyrazole (85.12 mg, 438.66 μmol), tert-butyl (3R)-3-[(2-bromothieno[3,2-c]pyridin-4-yl)-[2-fluoro-4-(1-methyltriazol-4-yl)benzoyl]amino]piperidine-1-carboxylate (100 mg, 146.22 μmol), K₂CO₃ (60.62 mg, 438.66 μmol) and Pd(dppf)Cl₂ (10.70 mg, 14.62 μmol) in dioxane (1.8 mL) and H₂O (0.2 mL) at 20° C. was degassed and purged with N₂. The reaction mixture was stirred at 100° C. for 6 h before it was diluted with water (15 mL) and extracted with EtOAc (3×5 mL). The combined organic layer was washed with brine (5 mL), dried over Na₂SO₄, filtered, and concentrated under reduced pressure. The residue was purified by Prep-TLC (ethyl acetate:methanol=10:1) to give tert-butyl (3R)-3-[[2-fluoro-4-(1-methyltriazol-4-yl)benzoyl]-[2-(1H-pyrazol-4-yl)thieno[3,2-c]pyridin-4-yl]amino]piperidine-1-carboxylate (20 mg, 20%) as a white solid.

### Step 2:

To a solution of tert-butyl (3R)-3-[[2-fluoro-4-(1-methyltriazol-4-yl)benzoyl]-[2-(1H-pyrazol-4-yl)thieno[3,2-c]pyridin-4-yl]amino]piperidine-1-carboxylate (10 mg, 14.93 μmol) in MeCN (1 mL) at 20° C. were added K₂CO₃ (6.19 mg, 44.80 μmol) and 2-chloroacetamide (2.09 mg, 22.40 μmol). The mixture was stirred at 60° C. for 2 h under N₂ Atmosphere. The reaction mixture was concentrated under reduced pressure to give tert-butyl (3R)-3-[[2-[1-(2-amino-2-oxo-ethyl)pyrazol-4-yl]thieno[3,2-c]pyridin-4-yl]-[2-fluoro-4-(1-methyltriazol-4-yl)benzoyl]amino]-piperidine-1-carboxylate (50 mg) as a brown solid, which was used directly in the next step.

### Step 3:

The title compound hydrochloride salt was obtained as an off-white solid by deprotection Method A used in Example 97. LC-MS: [M+H⁺]=560.4. ¹H NMR (400 MHZ, DMSO-d₆): δ 9.22-8.73 (m, 1H), 8.44 (s, 1H), 8.32-8.22 (m, 2H), 8.05-8.00 (m, 1H), 7.92 (br d, J=5.0 Hz, 1H), 7.75-7.66 (m, 2H), 7.39 (br d, J=11.0 Hz, 1H), 7.32 (br d, J=6.5 Hz, 2H), 7.24 (br d, J=7.0 Hz, 1H), 5.18-4.98 (m, 1H), 4.83 (br s, 2H), 4.00 (s, 3H), 3.71-3.70 (m, 2H), 3.45-3.44 (m, 1H), 3.17 (br d, J=7.9 Hz, 1H), 2.03 (br d, J=11.1 Hz, 1H), 1.91-1.73 (m, 2H), 1.25-1.09 (m, 1H).

### Synthesis of (R)-2-fluoro-N-(6-(3-(hydroxymethyl)phenyl)isoquinolin-1-yl)-4-(1-methyl-1H-1,2,3-triazol-4-yl)-N-(piperidin-3-yl)benzamide (Example 490)

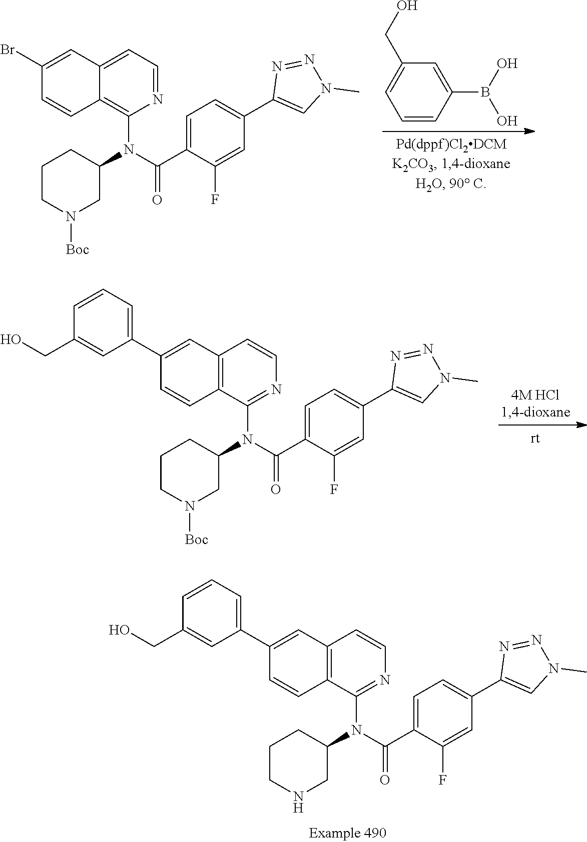

Example 290 was prepared according to the procedure described for Example 363 by substituting 4-pyridylboronic acid with (3-(hydroxymethyl)phenyl)boronic acid, provided the title compound as a white solid. LC-MS: [M+H⁺]=537.3. ¹H NMR (CD₃OD, 400 MHZ): δ 8.44-8.33 (m, 1H), 8.23-8.20 (m, 1H), 8.14-8.12 (m, 1H), 8.08-8.06 (m, 1H), 8.03-8.01 (m, 1H), 7.81 (d, J=5.6 Hz, 1H), 7.74-7.72 (m, 1H), 7.66-7.62 (m, 1H), 7.50-7.40 (m, 2H), 7.29-7.26 (m, 1H), 7.23-7.20 (m, 1H), 7.14-7.10 (m, 1H), 5.30-5.26 (m, 1H), 4.69 (s, 2H), 4.03 (s, 3H), 3.96-3.93 (m, 1H), 3.71-3.65 (m, 1H), 2.91-2.82 (m, 1H), 2.05-2.02 (m, 1H), 1.91-1.89 (m, 2H), 1.34-1.28 (m, 2H).

### Synthesis of (R)—N-(6-(2-aminopyridin-4-yl)isoquinolin-1-yl)-2-fluoro-4-(1-methyl-1H-1,2,3-triazol-4-yl)-N-(piperidin-3-yl)benzamide (Example 491)

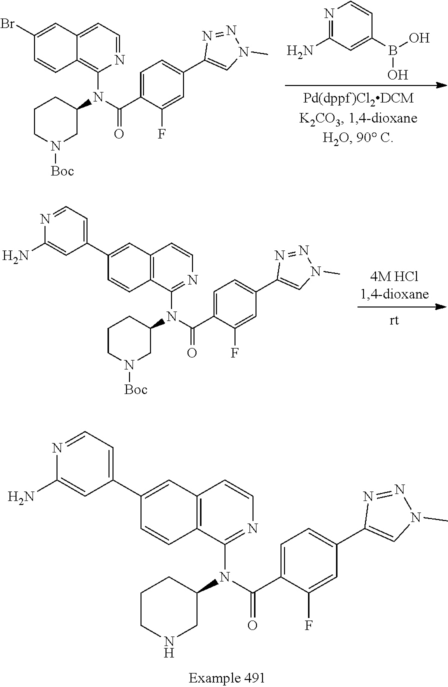 [SMILES: Cn1cc(-c2ccc(C(=O)N(c3nccc4cc(Br)ccc34)[C@@H]3CCCN(C(=O)OC(C)(C)C)C3)c(F)c2)nn1 ; Nc1cc(B(O)O)ccn1 ; Cn1cc(-c2ccc(C(=O)N(c3nccc4cc(-c5ccnc(N)c5)ccc34)[C@@H]3CCCN(C(=O)OC(C)(C)C)C3)c(F)c2)nn1 ; Cn1cc(-c2ccc(C(=O)N(c3nccc4cc(-c5ccnc(N)c5)ccc34)[C@@H]3CCCNC3)c(F)c2)nn1]

Example 291 was prepared according to the procedure described for Example 363 by substituting 4-pyridylboronic acid (2-aminopyridin-4-yl)boronic acid, provided the title compound as a white solid. LC-MS: [M+H⁺]=523.2. ¹H NMR (CD₃OD, 400 MHZ): δ 8.56-8.50 (m, 1H), 8.33-8.29 (m, 2H), 8.16-8.15 (m, 1H), 8.10-8.03 (m, 1H), 7.96-7.93 (m, 1H), 7.89 (d, J=5.6 Hz, 1H), 7.33-7.17 (m, 4H), 7.12-7.08 (m, 1H), 5.33-5.30 (m, 1H), 4.04 (s, 3H), 3.97-3.94 (m, 1H), 3.73-3.67 (m, 1H), 2.89-2.82 (m, 1H), 2.02-1.90 (m, 3H), 1.35-1.21 (m, 2H).

### Synthesis of (R)-2-fluoro-N-(6-(1-(2-hydroxyethyl)-1H-pyrazol-4-yl)isoquinolin-1-yl)-4-(1-methyl-1H-1,2,3-triazol-4-yl)-N-(piperidin-3-yl)benzamide (Example 492)

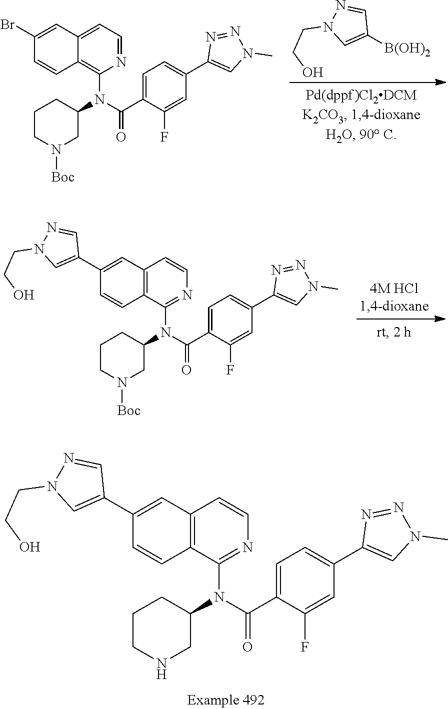

Example 292 was prepared according to the procedure described for Example 363 by substituting 4-pyridylboronic acid with (1-(2-hydroxyethyl)-1H-pyrazol-4-yl)boronic acid, provided the title compound hydrochloride salt as an off-white solid. LC-MS: [M+H⁺]=541.35. ¹H NMR (CD₃OD, 400 MHZ): δ 8.39-8.37 (m, 1H), 8.26-8.24 (m, 1H), 8.16-8.05 (m, 3H), 8.01-7.99 (m, 1H), 7.96-7.93 (m, 1H), 7.72-7.70 (m, 1H), 7.28-7.19 (m, 2H), 7.11-7.08 (m, 1H), 5.33-5.25 (m, 1H), 4.29-4.27 (m, 2H), 4.03 (s, 3H), 3.93-3.88 (m, 3H), 3.70-3.64 (m, 1H), 2.88-2.86 (m, 1H), 2.03-1.88 (m, 3H), 1.31-1.28 (m, 2H).

### Synthesis of (R)-2-fluoro-N-(6-(2-hydroxyphenyl)isoquinolin-1-yl)-4-(1-methyl-1H-1,2,3-triazol-4-yl)-N-(piperidin-3-yl)benzamide (Example 493)

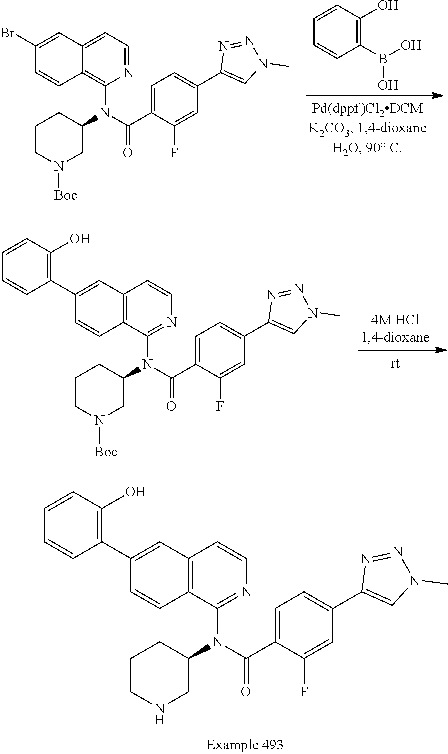

Example 293 was prepared according to the procedure described for Example 363 by substituting 4-pyridylboronic acid with (2-hydroxyphenyl)boronic acid, provided the title compound hydrochloride salt as an off-white solid. LC-MS: [M+H⁺]=523.2. ¹H NMR (CD₃OD, 400 MHZ): δ 8.42-8.36 (m, 1H), 8.16-8.06 (m, 3H), 8.01-7.99 (m, 1H), 7.81-7.76 (m, 1H), 7.35-7.31 (m, 2H), 7.28-7.21 (m, 2H), 7.17-7.13 (m, 1H), 6.96-6.90 (m, 2H), 5.26-5.20 (m, 1H), 4.05 (s, 3H), 3.95-3.93 (m, 1H), 3.69-3.66 (m, 1H), 2.89-2.85 (m, 1H), 2.08-1.88 (m, 3H), 1.34-1.26 (m, 2H).

### Synthesis of (R,E)-3-(1-(2-fluoro-4-(1-methyl-1H-1,2,3-triazol-4-yl)-N-(piperidin-3-yl)benzamido)isoquinolin-6-yl)acrylic acid (Example 494

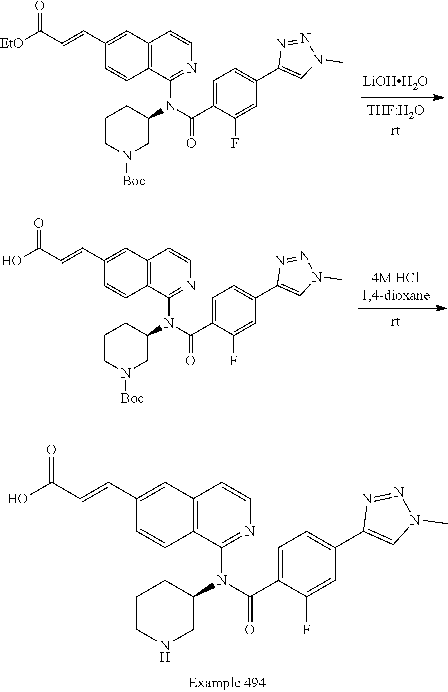

### Step 1:

To a solution of tert-butyl (R,E)-3-(N-(6-(3-ethoxy-3-oxoprop-1-en-1-yl)isoquinolin-1-yl)-2-fluoro-4-(1-methyl-1H-1,2,3-triazol-4-yl)benzamido)piperidine-1-carboxylate (80 mg, 0.127 mmol) in THF (5 mL) was added LiOH. H₂O (6.09 mg, 0.254 mmol) and H₂O (2 mL) at 0° C. and then the reaction was stirred at room temperature for 5 h. The reaction mixture was diluted with water (20 mL) and acidified with 1N aq. HCl to pH˜2 ands extracted with ethyl acetate (50 mL). The organic layer was washed with water (20 mL), brine solution (20 mL), dried by Na₂SO₄, and concentrated under reduced pressure. The residue was purified by prep HPLC (Conditions: Kinetex evo C-18 (250×21.2×5 μm), Mobile phase A: 0.1% formic acid in water (100%), Mobile phase B: 100% MeCN, Gradient: 0/60, 10/80, Flow: 15 mL/min, Dilution: MeCN+THF). The pure fractions were combined and concentrated to afford (R, E)-3-(1-(N-(1-(tert-butoxycarbonyl)piperidin-3-yl)-2-fluoro-4-(1-methyl-1H-1,2,3-triazol-4-yl)benzamido)-isoquinolin-6-yl)acrylic acid (25 mg, 29.9%) as a yellow solid. LC-MS: [M+H⁺]=601.3.

### Step 2:

The title compound hydrochloride salt was obtained as an off-white solid by deprotection Method A used in Example 97. LC-MS: [M+H⁺]=501.40. ¹H NMR (CD₃OD, 400 MHZ): δ 8.46 (d, J=5.6 Hz, 1H), 8.18-8.13 (m, 2H), 8.04-7.96 (m, 2H), 7.78-7.72 (m, 2H), 7.28-7.16 (m, 2H), 7.11-7.07 (m, 1H), 6.68 (d, J=16 Hz, 1H), 5.28-5.22 (m, 1H), 4.05 (s, 3H), 3.95-3.91 (m, 1H), 3.70-3.64 (m, 1H), 3.37-3.35 (m, 1H), 2.88-2.83 (m, 1H), 2.01-1.98 (m, 1H), 1.91-1.89 (m, 2H), 1.37-1.31 (m, 1H).

### Synthesis of (R)-2-fluoro-N-(6-(3-hydroxyphenyl)isoquinolin-1-yl)-4-(1-methyl-1H-1,2,3-triazol-4-yl)-N-(piperidin-3-yl)benzamide (Example 495)

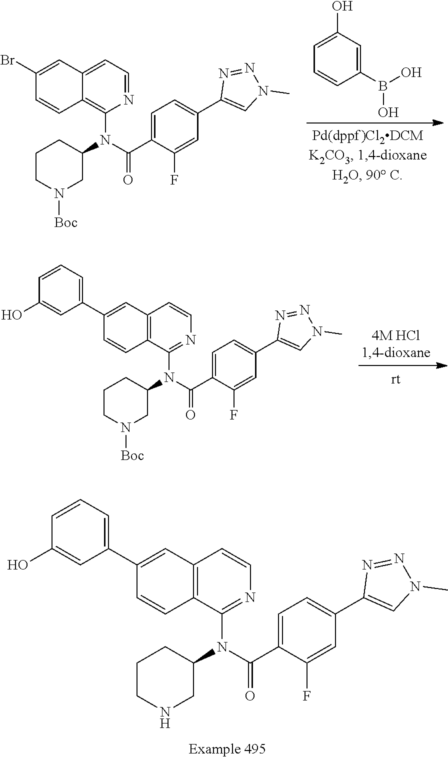

Example 293 was prepared according to the procedure described for Example 363 by substituting 4-pyridylboronic acid with (3-hydroxyphenyl)boronic acid, provided the title compound hydrochloride salt as an off-white solid. LC-MS: [M+H⁺]=523.3. ¹H NMR (CD₃OD, 400 MHZ): δ 8.43-8.42 (m, 1H), 8.20-8.14 (m, 1H), 8.13 (s, 1H), 8.02-7.95 (m, 2H), 7.80-7.79 (m, 1H), 7.29-7.21 (m, 2H), 7.20-7.18 (m, 2H), 7.19-7.10 (m, 2H), 6.87-6.83 (m, 1H), 5.30-5.23 (m, 1H), 4.03 (s, 3H), 3.96-3.93 (m, 1H), 3.70-3.65 (m, 1H), 2.89-2.85 (m, 1H), 2.05-2.01 (m, 1H), 1.91-1.89 (m, 2H), 1.31-1.28 (m, 2H).

### Synthesis of (R,E)-N-(6-(3-amino-3-oxoprop-1-en-1-yl)isoquinolin-1-yl)-2-fluoro-4-(1-methyl-1H-1,2,3-triazol-4-yl)-N-(piperidin-3-yl)benzamide (Example 496

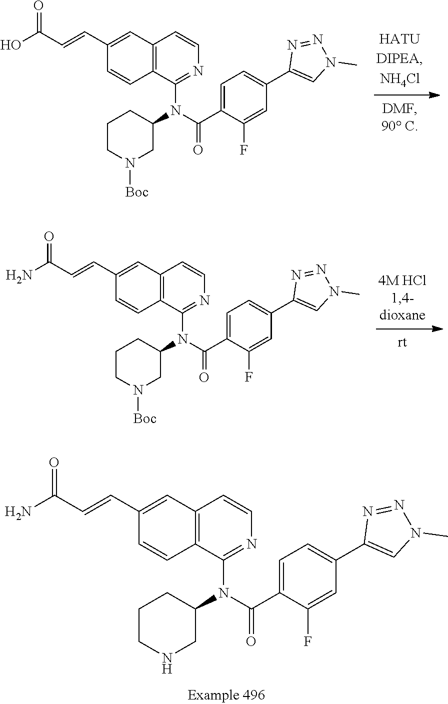 [SMILES: Cn1cc(-c2ccc(C(=O)N(c3nccc4cc(/C=C/C(=O)O)ccc34)[C@@H]3CCCN(C(=O)OC(C)(C)C)C3)c(F)c2)nn1 ; Cn1cc(-c2ccc(C(=O)N(c3nccc4cc(/C=C/C(N)=O)ccc34)[C@@H]3CCCN(C(=O)OC(C)(C)C)C3)c(F)c2)nn1 ; Cn1cc(-c2ccc(C(=O)N(c3nccc4cc(/C=C/C(N)=O)ccc34)[C@@H]3CCCNC3)c(F)c2)nn1]

### Step 1:

To a solution of (R,E)-3-(1-(N-(1-(tert-butoxycarbonyl)piperidin-3-yl)-2-fluoro-4-(1-methyl-1H-1,2,3-triazol-4-yl)benzamido)isoquinolin-6-yl)acrylic acid (80 mg, 0.133 mmol) in DMF (2 mL) was added DIPEA (0.070 mL, 0.400 mmol) followed by HATU (60.8 mg, 0.160 mmol) and ammonium chloride (21.37 mg, 0.400 mmol) at room temperature. The reaction mixture was then heated and stirred at 90° C. for 16 h. The reaction mixture was cooled to room temperature, diluted with ice cold water (20 mL) and extracted with EtOAc (2×50 mL). The organic layer was washed with brine (20 mL), dried over anhydrous sodium sulphate, and concentrated under reduced pressure to afford (80 mg). The residue was purified by Prep-HPLC (Conditions: Kinetex evo C-18 (250×21.2×5 μm), Mobile phase A: 0.1% formic acid in water (100%), Mobile phase B: 100% MeCN, Gradient: 0/40, 10/70, Flow: 15 mL/min, Dilution: MeCN+H₂O). The pure fractions were combined and concentrated under reduced pressure to afford tert-butyl (R,E)-3-(N-(6-(3-amino-3-oxoprop-1-en-1-yl)isoquinolin-1-yl)-2-fluoro-4-(1-methyl-1H-1,2,3-triazol-4-yl)benzamido)piperidine-1-carboxylate (22 mg, 27%) as an off-white solid. LC-MS: [M+H⁺]=600.3.

### Step 2:

The title compound hydrochloride salt was obtained as an off-white solid by deprotection Method A used in Example 97. LC-MS: [M+H⁺]=500.35. ¹H NMR (CD₃OD, 400 MHZ): δ 8.46 (d, J=6.0 Hz, 1H), 8.19-8.13 (m, 2H), 7.99-7.92 (m, 2H), 7.78-7.76 (m, 1H), 7.66-7.60 (m, 1H), 7.28-7.16 (m, 2H), 7.10-7.06 (m, 1H), 6.82 (d, J=16 Hz, 1H), 5.28-5.22 (m, 1H), 4.05 (s, 3H), 3.94-3.91 (m, 1H), 3.71-3.65 (m, 1H), 3.35-3.27 (m, 1H), 2.88-2.81 (m, 1H), 2.01-1.89 (m, 3H), 1.32-1.28 (m, 1H).

### Synthesis of N-(8-methylisoquinolin-1-yl)-3-(3-oxo-3-(pyridin-4-ylamino)propyl)-N—((R)-piperidin-3-yl)piperidine-1-carboxamide (Example 497)

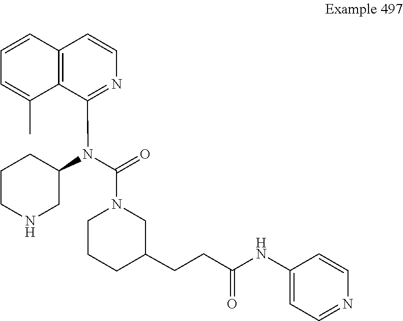 [SMILES: Cc1cccc2ccnc(N(C(=O)N3CCCC(CCC(=O)Nc4ccncc4)C3)[C@@H]3CCCNC3)c12]

Example 497 was prepared according to the procedure described for Example 243 by substituting 3-methyl-5-(piperidin-4-yl)isoxazole with 3-(piperidin-3-yl)-N-(pyridin-4-yl)propanamide and using 4M HCl in dioxane for removal of the tert-butyloxycarbonyl group, provided the title compound hydrochloride salt as an off-white solid. LC-MS: [M+H⁺]=501.3.

¹H NMR (CD₃OD, 400 MHZ): δ 8.62-8.61 (m, 2H), 8.41-8.28 (m, 1H), 8.18-8.12 (m, 2H), 7.85-7.84 (m, 2H), 7.70-7.68 (m, 1H), 7.62-7.53 (m, 1H), 4.97-4.82 (m, 1H), 3.95-3.90 (m, 1H), 3.68-3.65 (m, 2H), 2.96-2.91 (m, 3H), 2.72 (s, 3H), 2.34-2.17 (m, 5H), 1.85-1.82 (m, 3H), 1.61-1.48 (m, 1H), 1.34-1.30 (m, 2H), 1.21-0.80 (m, 3H), 0.29-0.25 (m, 1H).

### Synthesis of 3-(3-((1H-indazol-5-yl)amino)-3-oxopropyl)-N-(8-methylisoquinolin-1-yl)-N—((R)-piperidin-3-yl)piperidine-1-carboxamide (Example 498)

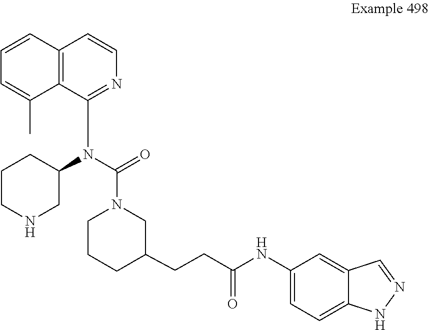 [SMILES: Cc1cccc2ccnc(N(C(=O)N3CCCC(CCC(=O)Nc4ccc5[nH]ncc5c4)C3)[C@@H]3CCCNC3)c12]

Example 498 was prepared according to the procedure described for Example 243 by substituting 3-methyl-5-(piperidin-4-yl)isoxazole with N-(1H-indazol-5-yl)-3-(piperidin-3-yl)propanamide and using 4M HCl in dioxane for removal of the tert-butyloxycarbonyl group, provided the title compound hydrochloride salt as an off-white solid. LC-MS: [M+H⁺]=540.3. ¹H NMR (CD₃OD, 400 MHZ): δ 8.56 (s, 1H), 8.42-8.30 (m, 2H), 7.93-7.93 (m, 2H), 7.76-7.48 (m, 4H), 4.89-4.63 (m, 1H), 4.02-3.92 (m, 1H), 3.80-3.67 (m, 1H), 3.64-3.43 (m, 2H), 3.18-3.00 (m, 1H), 3.00-2.88 (m, 2H), 2.74 (s, 3H), 2.72-2.54 (m, 1H), 2.52-1.74 (m, 7H), 1.72-1.48 (m, 1H), 1.46-1.24 (m, 2H), 1.22-1.14 (m, 1H), 0.98-0.78 (m, 2H).

### Synthesis of 3-(3-((1H-indazol-6-yl)amino)-3-oxopropyl)-N-(8-methylisoquinolin-1-yl)-N—((R)-piperidin-3-yl)piperidine-1-carboxamide (Example 499)

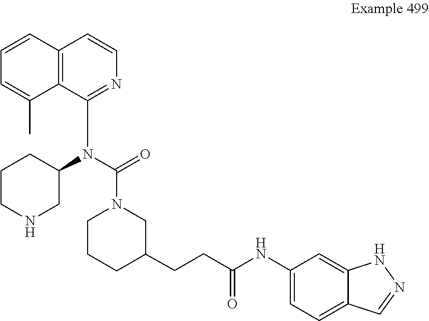 [SMILES: Cc1cccc2ccnc(N(C(=O)N3CCCC(CCC(=O)Nc4ccc5cn[nH]c5c4)C3)[C@@H]3CCCNC3)c12]

Example 499 was prepared according to the procedure described for Example 243 by substituting 3-methyl-5-(piperidin-4-yl)isoxazole with N-(1H-indazol-6-yl)-3-(piperidin-3-yl)propanamide and using 4M HCl in dioxane for removal of the tert-butyloxycarbonyl group, provided the title compound hydrochloride salt as an off-white solid. LC-MS: [M+H⁺]=540.3.

¹H NMR (CD₃OD, 400 MHZ): δ 8.58 (s, 1H), 8.42-8.27 (m, 2H), 7.93-7.90 (m, 1H), 7.85-7.78 (m, 2H), 7.68-7.51 (m, 2H), 7.41-7.24 (m, 1H), 4.86-4.70 (m, 1H), 3.95-3.89 (m, 1H), 3.75-3.56 (m, 2H), 3.24-2.89 (m, 3H), 2.70 (s, 3H), 2.27-1.81 (m, 8H), 1.64-1.53 (m, 1H), 1.35-1.31 (m, 2H), 1.14-1.12 (m, 1H), 0.92-0.83 (m, 2H), 0.23-0.14 (m, 1H).

### Synthesis of (E)-3-[4-[[2-fluoro-4-(triazolo[4,5-b]pyridin-3-yl)benzoyl]-[(3R)-3-piperidyl]amino]thieno[3,2-c]pyridin-2-yl]prop-2-enoic acid (Example 500)

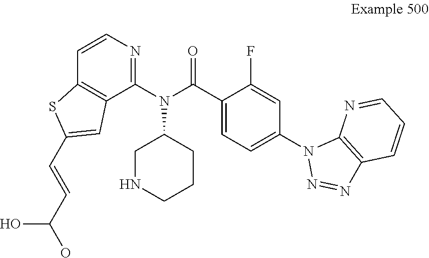 [SMILES: O=C(c1ccc(-n2nnc3cccnc32)cc1F)N(c1nccc2sc(/C=C/C(O)O)cc12)[C@@H]1CCCNC1]

Example 500 was prepared using the procedure described for Example 586, by substituting tert-butyl (3R)-3-[(2-bromothieno[3,2-c]pyridin-4-yl)-[2-fluoro-4-(1-methyltriazol-4-yl)benzoyl]amino]piperidine-1-carboxylate with (R)-4-(3H-[1,2,3]triazolo[4,5-b]pyridin-3-yl)-N-(2-bromothieno[3,2-c]pyridin-4-yl)-2-fluoro-N-(piperidin-3-yl)benzamide, provided the title compound as an off-white solid. LC-MS: [M+H⁺]=544.4. ¹H NMR (400 MHz, CD₃OD): δ 8.77 (br d, J=3.6 Hz, 1H), 8.50 (d, J=8.3 Hz, 1H), 8.34 (br d, J=5.4 Hz, 1H), 8.11 (br d, J=10.9 Hz, 1H), 8.01 (br d, J=7.8 Hz, 1H), 7.82 (br d, J=5.5 Hz, 1H), 7.64-7.57 (m, 2H), 7.53 (dd, J=4.6, 8.3 Hz, 1H), 7.49-7.39 (m, 1H), 6.40 (br d, J=15.4 Hz, 1H), 5.22-5.07 (m, 1H), 3.93-3.76 (m, 1H), 3.68-3.54 (m, 1H), 3.18-2.76 (m, 2H), 2.18-1.81 (m, 4H), 1.52-1.27 (m, 1H).

### Synthesis of ethyl (E)-3-[4-[[2-fluoro-4-(triazolo[4,5-b]pyridin-3-yl)benzoyl]-[(3R)-3-piperidyl]amino]thieno[3,2-c]pyridin-2-yl]prop-2-enoate (Example 501)

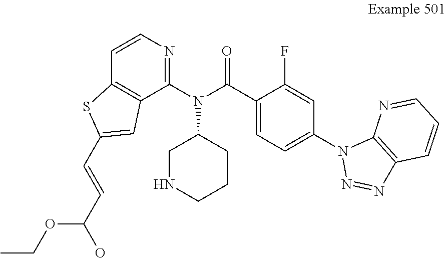 [SMILES: CCOC(O)/C=C/c1cc2c(N(C(=O)c3ccc(-n4nnc5cccnc54)cc3F)[C@@H]3CCCNC3)nccc2s1]

Example 501 was prepared according to the procedure described for Example 486 by substituting tert-butyl (3R)-3-[(2-bromothieno[3,2-c]pyridin-4-yl)-[2-fluoro-4-(1-methyltriazol-4-yl)benzoyl]amino]piperidine-1-carboxylate with tert-butyl (R)-3-(4-(3H-[1,2,3]triazolo[4,5-b]pyridin-3-yl)-N-(2-bromothieno[3,2-c]pyridin-4-yl)-2-fluorobenzamido)piperidine-1-carboxylate, provided the title compound as an off-white solid. LC-MS: [M+H⁺]=572.3. ¹H NMR (400 MHZ, CD₃OD): δ 8.77 (br s, 1H), 8.51 (br d, J=8.1 Hz, 1H), 8.37 (br d, J=1.9 Hz, 1H), 8.08 (br d, J=10.1 Hz, 1H), 8.03-7.91 (m, 2H), 7.81 (br s, 2H), 7.59-7.49 (m, 1H), 7.41 (br d, J=5.8 Hz, 1H), 6.39 (br d, J=15.9 Hz, 1H), 4.72-4.50 (m, 1H), 4.28 (q, J=7.1 Hz, 2H), 3.63-3.50 (m, 1H), 3.01-2.88 (m, 2H), 2.46-2.30 (m, 2H), 2.09-1.93 (m, 1H), 1.81-1.58 (m, 2H), 1.34 (t, J=7.1 Hz, 3H).

### Synthesis of 4-[4-[[2-fluoro-4-(triazolo[4,5-b]pyridin-3-yl)benzoyl]-[(3R)-3-piperidyl]amino]thieno[3,2-c]pyridin-2-yl]thiazole-2-carboxylic acid (Example 502)

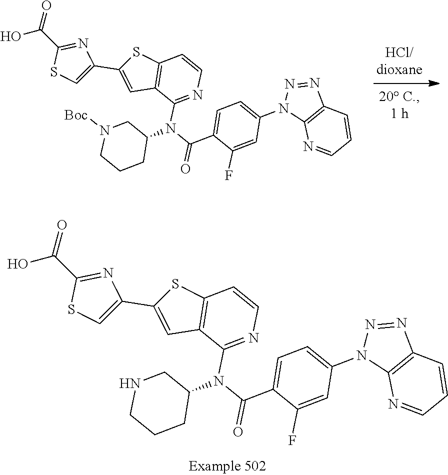 [SMILES: CC(C)(C)OC(=O)N1CCC[C@@H](N(C(=O)c2ccc(-n3nnc4cccnc43)cc2F)c2nccc3sc(-c4csc(C(=O)O)n4)cc23)C1 ; O=C(O)c1nc(-c2cc3c(N(C(=O)c4ccc(-n5nnc6cccnc65)cc4F)[C@@H]4CCCNC4)nccc3s2)cs1]

A solution of 4-[4-[[(3R)-1-tert-butoxycarbonyl-3-piperidyl]-[2-fluoro-4-(triazolo[4,5-b]pyridin-3-yl)benzoyl]amino]thieno[3,2-c]pyridin-2-yl]thiazole-2-carboxylic acid (30 mg, 21.41 μmol) in 4M HCl in dioxane (2 mL) was stirred at 20° C. for 1 hour. The reaction mixture was concentrated, and the residue was purified by prep-HPLC (HCl condition; column: Phenomenex Luna C18 100*40 mm*5 μm; mobile phase: [H₂O (0.04% HCl)-MeCN]; gradient: 10%-50% B over 8.0 min) and the fraction was lyophilized to give 4-[4-[[2-fluoro-4-(triazolo[4,5-b]pyridin-3-yl)benzoyl]-[(3R)-3-piperidyl]amino]thieno[3,2-c]pyridin-2-yl]thiazole-2-carboxylic acid (9.5 mg, 68%) as an off-white solid. LC-MS: [M+H⁺]=601.3. ¹H NMR (400 MHZ, DMSO-d₆): δ 9.03-8.87 (m, 2H), 8.85 (s, 1H), 8.80 (br d, J=4.4 Hz, 1H), 8.70-8.63 (m, 1H), 8.42-8.35 (m, 1H), 8.32-8.24 (m, 1H), 8.11-8.03 (m, 1H), 7.98 (br d, J=10.5 Hz, 1H), 7.92-7.83 (m, 1H), 7.65-7.48 (m, 2H), 5.26-4.89 (m, 1H), 3.82-3.71 (m, 2H), 2.86-2.72 (m, 2H), 2.16-2.08 (m, 1H), 1.95-1.73 (m, 3H), 1.28-1.17 (m, 1H).

### Synthesis of 2-fluoro-N-[(3R)-3-piperidyl]-N-[2-[2-(4-pyridyl)ethyl]thieno[3,2-c]pyridin-4-yl]-4-(triazolo[4,5-b]pyridin-3-yl)benzamide (Example 503)

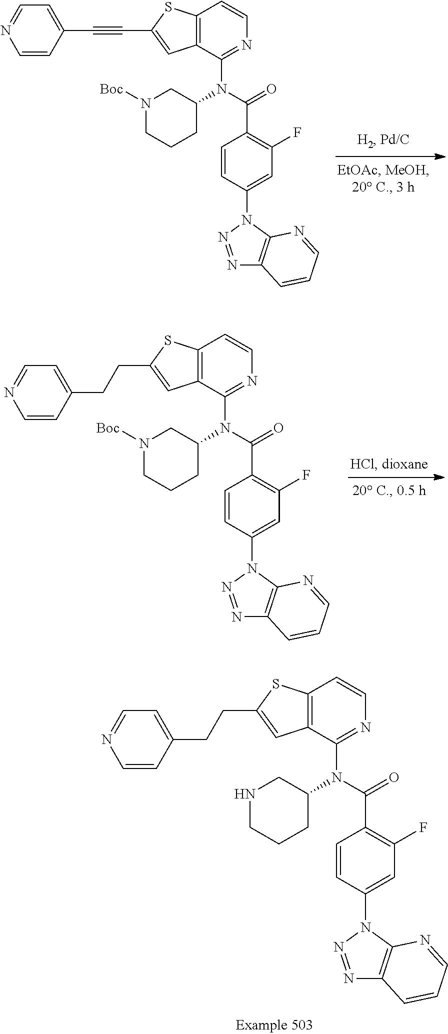 [SMILES: CC(C)(C)OC(=O)N1CCC[C@@H](N(C(=O)c2ccc(-n3nnc4cccnc43)cc2F)c2nccc3sc(C#Cc4ccncc4)cc23)C1 ; CC(C)(C)OC(=O)N1CCC[C@@H](N(C(=O)c2ccc(-n3nnc4cccnc43)cc2F)c2nccc3sc(CCc4ccncc4)cc23)C1 ; O=C(c1ccc(-n2nnc3cccnc32)cc1F)N(c1nccc2sc(CCc3ccncc3)cc12)[C@@H]1CCCNC1]

### Step 1:

To a mixture of Pd/C (141.95 mg, 133.38 μmol, 10% purity) in ethyl acetate (2 mL) was added tert-butyl (3R)-3-[[2-fluoro-4-(triazolo[4,5-b]pyridin-3-yl)benzoyl]-[2-[2-(4-pyridyl) ethynyl]thieno[3,2-c]pyridin-4-yl]amino]piperidine-1-carboxylate (30 mg, 44.46 μmol) in MeOH (0.5 mL), and the mixture was degassed and purged with H₂ 3 times. The mixture was stirred under H₂ (15 Psi) at 20° C. for 3 h. The reaction mixture was filtered and concentrated to give tert-butyl (3R)-3-[[2-fluoro-4-(triazolo[4,5-b]pyridin-3-yl)benzoyl]-[2-[2-(4-pyridyl)ethyl]thieno[3,2-c]pyridin-4-yl]amino]piperidine-1-carboxylate (26 mg, 86%) as a yellow solid, which was used directly in the next step.

### Step 2:

The title compound hydrochloride salt was obtained as an off-white solid by deprotection Method A used in Example 97. LC-MS: [M+H⁺]=579.4. ¹H NMR (400 MHZ, CD₃OD): δ 8.80 (br d, J=5.3 Hz, 3H), 8.55 (br d, J=8.4 Hz, 1H), 8.34 (br d, J=5.3 Hz, 1H), 8.15-7.93 (m, 4H), 7.83 (br s, 1H), 7.57 (br dd, J=4.6, 8.2 Hz, 1H), 7.48-7.33 (m, 2H), 5.29-5.12 (m, 1H), 3.95-3.82 (m, 1H), 3.68-3.43 (m, 5H), 3.10-2.69 (m, 2H), 2.15-1.81 (m, 3H), 1.44-1.23 (m, 1H).

### Synthesis of (R,Z)-2-fluoro-N-(2-(3-hydroxyprop-1-en-1-yl)thieno[3,2-c]pyridin-4-yl)-4-(1-methyl-1H-1,2,3-triazol-4-yl)-N-(piperidin-3-yl)benzamide (Example 504)

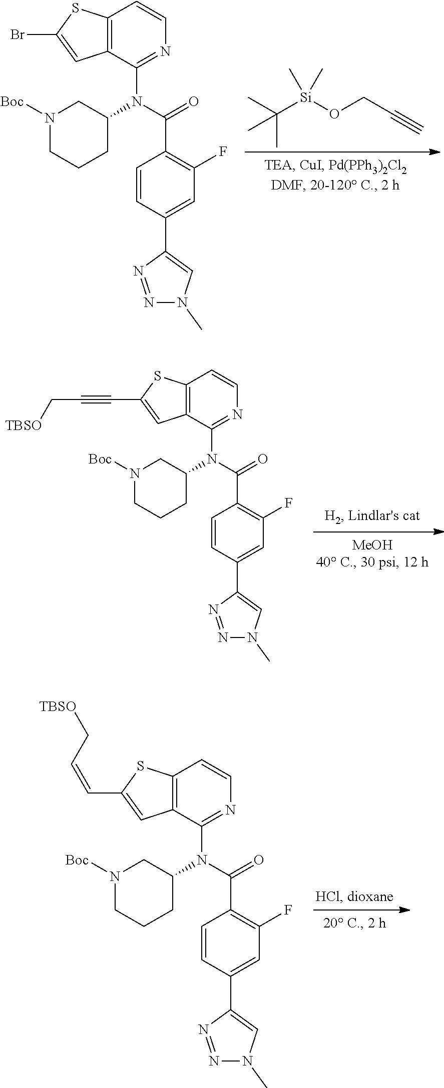

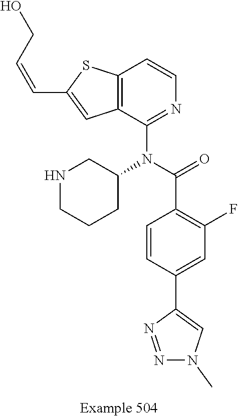 [SMILES: Cn1cc(-c2ccc(C(=O)N(c3nccc4sc(/C=C\CO)cc34)[C@@H]3CCCNC3)c(F)c2)nn1]

### Step 1:

To a solution of tert-butyl (3R)-3-[(2-bromothieno[3,2-c]pyridin-4-yl)-[2-fluoro-4-(1-methyltriazol-4-yl)benzoyl]amino]piperidine-1-carboxylate (0.15 g, 243.70 μmol) in DMF (1.5 mL) was added tert-butyl-dimethyl-prop-2-ynoxy-silane (124.52 mg, 731.09 μmol), TEA (73.98 mg, 731.09 μmol), CuI (13.92 mg, 73.11 μmol), and dichloropalladiumtriphenylphosphane (17.11 mg, 24.37 μmol) at 20° C. under N₂. The mixture was stirred at 120° C. for 2 h before it was filtrated, and the filtrate was diluted with water (5 mL) and extracted with ethyl acetate (3×5 mL). The combined organic layer was washed with brine, dried over Na₂SO₄, filtered, and concentrated. The residue was purified by Prep-HPLC (column: Phenomenex Luna C18 100*40 mm*5 μm; mobile phase: [H₂O (0.04% HCl)-MeCN]; gradient: 75%-95% B over 8.0 min). The pure fraction was lyophilized to give tert-butyl (3R)-3-[[2-[3-[tert-butyl(dimethyl)silyl]oxyprop-1-ynyl]thieno[3,2-c]pyridin-4-yl]-[2-fluoro-4-(1-methyltriazol-4-yl)benzoyl]amino]piperidine-1-carboxylate (0.05 g, 26%) as a light brown solid.

Step 2: To a solution of tert-butyl (3R)-3-[[2-[3-[tert-butyl(dimethyl)silyl]oxyprop-1-ynyl]thieno[3,2-c]pyridin-4-yl]-[2-fluoro-4-(1-methyltriazol-4-yl)benzoyl]amino]piperidine-1-carboxylate (0.025 g, 31.92 μmol) in MeOH (0.5 mL) was added calcium palladium carbonate (65.91 mg, 31.92 μmol 10 wt %) at 20° C. was purged with H₂ and then stirred under H₂ (30 psi) at 40° C. for 24 h. The mixture was filtered through a celite pad, and the filtrate was concentrated. The residue was purified by Prep-HPLC (column: Waters Xbridge Prep OBD C18 150*40 mm*10 μm; mobile phase: [H₂O (10 mM NH₄HCO₃)-MeCN]; gradient: 80%-98% B over 8.0 min). The pure fraction was lyophilized to give tert-butyl (3R)-3-[[2-[(Z)-3-[tert-butyl(dimethyl)silyl]oxyprop-1-enyl]thieno[3,2-c]pyridin-4-yl]-[2-fluoro-4-(1-methyltriazol-4-yl)benzoyl]amino]piperidine-1-carboxylate (0.007 g, 28%) as a white solid.

### Step 3:

The title compound hydrochloride salt was obtained as an off-white solid by deprotection Method A used in Example 97. LC-MS: [M+H⁺]=493.3. ¹H NMR (400 MHZ, DMSO-d₆): δ 9.05-8.80 (m, 2H), 8.46 (br s, 1H), 8.34-8.29 (m, 1H), 7.98-7.96 (m, 1H), 7.48-7.30 (m, 3H), 7.20-7.15 (m, 1H), 6.74-6.66 (m, 1H), 6.03-5.97 (m, 1H), 5.11-5.06 (m, 1H), 4.36-4.35 (m, 2H), 4.02 (s, 3H), 3.53-3.47 (m, 1H), 3.31-3.17 (m, 2H), 2.72-2.67 (m, 1H), 1.95-1.73 (m, 3H), 1.14-1.10 (m, 1H).

### Synthesis of 2-fluoro-4-(1-methyltriazol-4-yl)-N-[(3R)-3-piperidyl]-N-[2-(4-pyridyl)thieno[2,3-c]pyridin-7-yl]benzamide (Example 505)

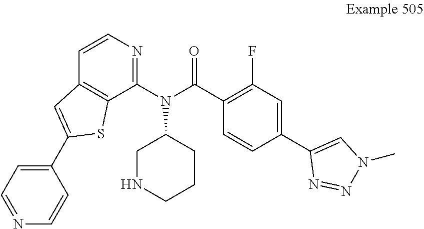 [SMILES: Cn1cc(-c2ccc(C(=O)N(c3nccc4cc(-c5ccncc5)sc34)[C@@H]3CCCNC3)c(F)c2)nn1]

Example 505 was prepared according to the procedure described for Example 384 by substituting 2-fluoro-4-(triazolo[4,5-b]pyridin-3-yl)benzoyl chloride with 2-fluoro-4-(1-methyltriazol-4-yl)benzoyl chloride, provided the title compound hydrochloride salt as an off-white solid. LC-MS: [M+H⁺]=514.3. ¹H NMR (400 MHZ, DMSO-d₆): δ 8.78-8.66 (m, 2H), 8.45 (br s, 2H), 8.25-8.11 (m, 1H), 7.88-7.73 (m, 3H), 7.57-7.08 (m, 3H), 4.81-4.38 (m, 1H), 4.11-3.93 (m, 3H), 2.87-2.63 (m, 2H), 2.24-1.87 (m, 3H), 1.72-1.38 (m, 3H), 1.30-1.06 (m, 1H).

### Synthesis of N-(2-bromothieno[3, 2-c]pyridin-4-yl)-2-fluoro-4-(1-methyltriazol-4-yl)-N-[(3R)-3-piperidyl] benzamide (Example 506)

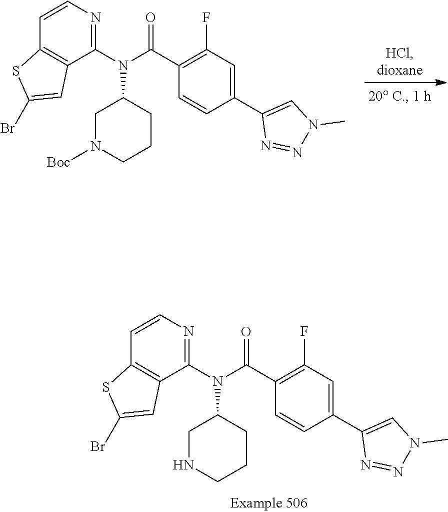 [SMILES: Cn1cc(-c2ccc(C(=O)N(c3nccc4sc(Br)cc34)[C@@H]3CCCN(C(=O)OC(C)(C)C)C3)c(F)c2)nn1 ; Cn1cc(-c2ccc(C(=O)N(c3nccc4sc(Br)cc34)[C@@H]3CCCNC3)c(F)c2)nn1]

A solution of tert-butyl (3R)-3-[(2-bromothieno[3,2-c]pyridin-4-yl)-[2-fluoro-4-(1-methyltriazol-4-yl)benzoyl]amino]piperidine-1-carboxylate (20 mg, 32.49 μmol) in 4M HCl in dioxane (1 mL) at 20° C. was stirred for 30 min. The mixture was concentrated under reduced pressure. The residue was purified by Prep-HPLC (column: Phenomenex Luna C18 100*40 mm*5 μm; mobile phase: [H₂O (0.04% HCl)-MeCN]; gradient: 5%-35% B over 8.0 min). The pure fraction was lyophilized to give N-(2-bromothieno [3, 2-c] pyridin-4-yl)-2-fluoro-4-(1-methyltriazol-4-yl)-N-[(3R)-3-piperidyl] benzamide (6.5 mg, 38%) as a white solid. LC-MS: [M+H⁺]=515.2. ¹H NMR (400 MHZ, DMSO-d₆): δ 9.29-8.83 (m, 2H), 8.54 (br s, 1H), 8.42 (br d, J=5.0 Hz, 1H), 8.04 (br d, J=4.0 Hz, 1H), 7.87 (br s, 1H), 7.55-7.34 (m, 2H), 7.23 (br d, J=6.3 Hz, 1H), 5.09 (br s, 1H), 4.09 (s, 3H), 3.77-3.70 (m, 1H), 3.38 (br s, 1H), 3.23 (br s, 1H), 2.91-2.75 (m, 1H), 2.02 (br s, 1H), 1.96-1.75 (m, 2H), 1.21 (br s, 1H).

### Synthesis of 2-fluoro-4-(1-methyltriazol-4-yl)-N-[(3R)-3-piperidyl]-N-[2-(2-pyridyl) thieno [3, 2-c] pyridin-4-yl] benzamide (Example 507)

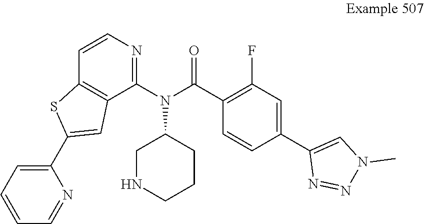 [SMILES: Cn1cc(-c2ccc(C(=O)N(c3nccc4sc(-c5ccccn5)cc34)[C@@H]3CCCNC3)c(F)c2)nn1]

Example 507 was prepared according to the procedure described for Example 363 by substituting 4-pyridylboronic acid with 2-pyridylboronic acid, provided the title compound hydrochloride salt as an off-white solid. LC-MS: [M+H⁺]=514.3. ¹H NMR (400 MHZ, DMSO-d₆): δ 8.69-8.56 (m, 1H), 8.49-8.18 (m, 4H), 8.02-7.88 (m, 2H), 7.51-7.39 (m, 1H), 7.38-7.11 (m, 3H), 4.80-4.54 (m, 1H), 4.10-3.91 (m, 3H), 2.96-2.72 (m, 2H), 2.29-2.10 (m, 2H), 2.05-1.89 (m, 1H), 1.84-1.61 (m, 1H), 1.61-1.36 (m, 2H), 1.10-0.70 (m, 1H).

### Synthesis of 2-fluoro-4-(1-methyltriazol-4-yl)-N-[(3R)-3-piperidyl]-N-[2-(3-pyridyl) thieno [3, 2-c] pyridin-4-yl] benzamide (Example 508)

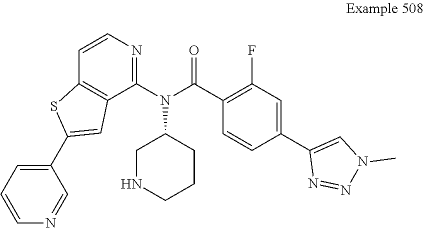 [SMILES: Cn1cc(-c2ccc(C(=O)N(c3nccc4sc(-c5cccnc5)cc34)[C@@H]3CCCNC3)c(F)c2)nn1]

Example 508 was prepared according to the procedure described for Example 363 by substituting 4-pyridylboronic acid with 3-pyridylboronic acid, provided the title compound hydrochloride salt as an off-white solid. LC-MS: [M+H⁺]=514.3. ¹H NMR (400 MHZ, DMSO-d₆): δ 9.30-8.98. (m, 3H), 8.78-8.71 (m, 1H), 8.47-8.37 (m, 3H), 8.12-8.10 (m, 1H), 7.72-7.70 (m, 1H), 7.43-7.17 (m, 2H), 5.18-4.97 (m, 1H), 4.09 (s, 3H), 3.78-3.70 (m, 1H), 3.39-3.33 (m, 1H), 3.20-3.15 (m, 1H), 2.75-2.64 (m, 1H), 2.08-2.06 (m, 1H), 1.90-1.73 (m, 2H), 1.18-1.04 (m, 1H).

### Synthesis of N-[2-(3-aminophenyl)thieno[3,2-c]pyridin-4-yl]-2-fluoro-4-(1-methyltriazol-4-yl)-N-[(3R)-3-piperidyl]benzamidezamide (Example 509)

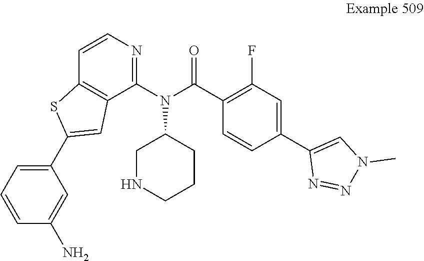 [SMILES: Cn1cc(-c2ccc(C(=O)N(c3nccc4sc(-c5cccc(N)c5)cc34)[C@@H]3CCCNC3)c(F)c2)nn1]

Example 509 was prepared according to the procedure described for Example 363 by substituting 4-pyridylboronic acid with (3-aminophenyl)boronic acid, provided the title compound hydrochloride salt as an off-white solid. LC-MS: [M+H⁺]=528.3. ¹H NMR (400 MHz, CD3OD): δ 8.40 (br d, J=5.4 Hz, 1H), 8.18 (s, 1H), 7.98-7.86 (m, 2H), 7.86-7.74 (m, 2H), 7.70 (t, J=7.9 Hz, 1H), 7.48 (br d, J=8.1 Hz, 1H), 7.39-7.24 (m, 2H), 7.18 (br d, J=7.3 Hz, 1H), 5.39-5.19 (m, 1H), 4.06 (s, 3H), 3.90 (br d, J=10.9 Hz, 1H), 3.76-3.64 (m, 1H), 3.60-3.52 (m, 1H), 3.37 (br s, 1H), 2.98-2.80 (m, 1H), 2.16-1.79 (m, 3H), 1.49-1.31 (m, 1H).

### Synthesis of 2-fluoro-4-(1-methyltriazol-4-yl)-N-(2-phenylthieno[3,2-c]pyridin-4-yl)-N-[(3R)-3-piperidyl]benzamide (Example 510)

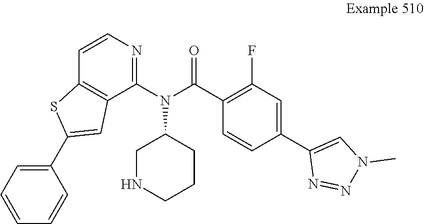 [SMILES: Cn1cc(-c2ccc(C(=O)N(c3nccc4sc(-c5ccccc5)cc34)[C@@H]3CCCNC3)c(F)c2)nn1]

Example 510 was prepared according to the procedure described for Example 363 by substituting 4-pyridylboronic acid with phenylboronic acid, provided the title compound hydrochloride salt as a white solid. LC-MS: [M+H⁺]=513.3. ¹H NMR (400 MHz, DMSO-d6): δ 9.04-8.80 (m, 2H), 8.42 (s, 1H), 8.37-8.30 (m, 1H), 8.03-7.95 (m, 2H), 7.85 (br d, J=7.5 Hz, 2H), 7.58-7.44 (m, 4H), 7.38 (br d, J=10.3 Hz, 1H), 7.34-7.28 (m, 1H), 7.26-7.18 (m, 1H), 5.19-4.91 (m, 1H), 3.99 (s, 3H), 3.77-3.68 (m, 1H), 3.34 (br s, 1H), 3.23-3.13 (m, 1H), 2.75-2.66 (m, 1H), 2.05 (br d, J=9.3 Hz, 1H), 1.95-1.66 (m, 3H), 1.21-1.07 (m, 1H).

### Synthesis of 2-fluoro-4-(1-methyltriazol-4-yl)-N-[(3R)-3-piperidyl]-N-[2-(3-thienyl)thieno[3,2-c]pyridin-4-yl]benzamide (Example 511)

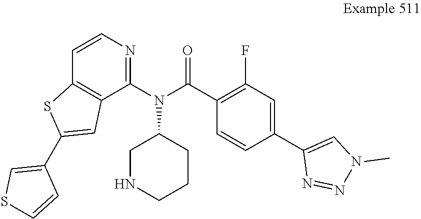 [SMILES: Cn1cc(-c2ccc(C(=O)N(c3nccc4sc(-c5ccsc5)cc34)[C@@H]3CCCNC3)c(F)c2)nn1]

Example 511 was prepared according to the procedure described for Example 363 by substituting 4-pyridylboronic acid with 3-thienylboronic acid, provided the title compound hydrochloride salt as a white solid. LC-MS: [M+H⁺]=519.3. ¹H NMR (400 MHZ, DMSO-d6): δ 9.01-8.77 (m, 2H), 8.42 (s, 1H), 8.33-8.27 (m, 1H), 8.08-8.00 (m, 1H), 7.96 (br d, J=5.3 Hz, 1H), 7.88-7.83 (m, 1H), 7.76 (br dd, J=2.8, 4.8 Hz, 1H), 7.69 (br d, J=4.8 Hz, 1H), 7.37 (br d, J=11.3 Hz, 1H), 7.31 (br d, J=7.9 Hz, 1H), 7.27-7.19 (m, 1H), 5.18-4.89 (m, 1H), 4.00 (s, 3H), 3.75-3.66 (m, 1H), 3.23-3.14 (m, 1H), 2.82-2.64 (m, 2H), 2.03 (br d, J=11.3 Hz, 1H), 1.95-1.66 (m, 3H), 1.21-1.06 (m, 1H).

### Synthesis of (R)-3-(4-hydroxybut-1-yn-1-yl)-N-(8-methylisoquinolin-1-yl)-N-(piperidin-3-yl)benzamide (Example 512)

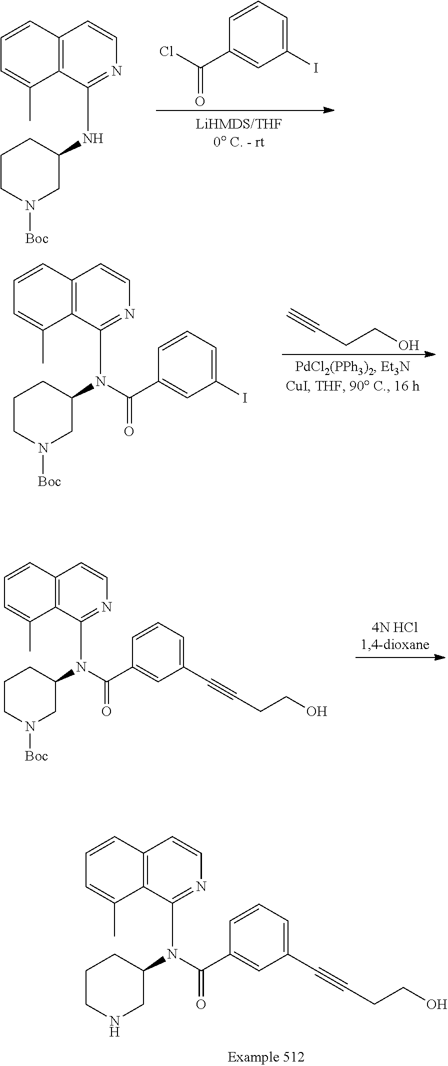

### Step 1:

To a stirred solution of tert-butyl (R)-3-((8-methylisoquinolin-1-yl) amino) piperidine-1-carboxylate (2.5 g, 7.32 mmol) in THF (25 mL) was added LiHMDS in THF (3.68 g, 21.96 mmol) at 0° C. and stirred for 10 mins. Then, 3-iodobenzoyl chloride (3.90 g, 14.64 mmol) in THF (20 mL) was added to reaction mixture and stirred for 1 h. The reaction mixture was quenched with Ice cold water (100 mL) and extracted with EtOAc (2×100 mL). The organic layers were dried over Na₂SO₄ and concentrated under reduced pressure. The obtained crude compound was purified by Combi-flash using eluted with 20% EtOAc in Hexane (3.5 g). The residue was further purified by prep HPLC (Conditions: X-SELECT C-18 (250×19×5 μm), Mobile phase A: 0.1% formic acidformic acid in water (100%), Mobile phase B: 100% MeCN, Gradient: 0/70, 10/90, Flow: 15 mL/min, Dilution: MeCN+H₂O+THF). The pure fractions were concentrated under reduced pressure to affords tert-butyl (R)-3-(3-iodo-N-(8-methylisoquinolin-1-yl) benzamido) piperidine-1-carboxylate (700 mg, 18%) as yellow solid. LC-MS: [M+1⁺]=572.1.

### Step 2:

To a solution of tert-butyl (R)-3-(3-iodo-N-(8-methylisoquinolin-1-yl)benzamido)piperidine-1-carboxylate (100 mg, 0.175 mmol) in THF (2 mL) were added Et₃N (0.049 ml, 0.350 mmol) & copper(I) iodide (16.66 mg, 0.087 mmol) & 3-butynol (24.53 mg, 0.350 mmol) at room temperature. The reaction mixture was degassed with nitrogen for 5 minutes. Then, PdCl₂(pph₃)₂ (13.80 mg, 0.017 mmol)) was added to reaction mixture and stirred at 90° C. for 16 h. The reaction mixture was cooled and diluted with water (10 mL) and extracted with EtOAc (2×10 mL). The combined organic layer was washed with brine, dried over Na₂SO₄, and concentrated under reduced pressure. The residue was purified by Combi-flash (50% EtOAc in hexane) first and then was further purified by Prep-HPLC (Conditions: Luna OMEGA C-18 (250×21.2×5 μm), 0.1% formic acidformic acid in water, Mobile phase B: MeCN (100%), T/% B: 0/60, 10/90, MeCN+H₂O+THF. The pure fractions were concentrated under reduced pressure to afford tert-butyl (R)-3-(3-(4-hydroxybut-1-yn-1-yl)-N-(8-methylisoquinolin-1-yl) benzamido) piperidine-1-carboxylate (40 mg, 56%) as an off-white solid. LC-MS: [M+H⁺]=514.3.

### Step 3:

The title compound hydrochloride salt was obtained as an off-white solid by deprotection Method A used in Example 97. LC-MS: [M+H⁺]=450.2. ¹H NMR (CD₃OD, 400 MHZ): δ 8.48-8.44 (m, 1H), 7.81-7.76 (m, 1H), 7.70-7.66 (m, 1H), 7.50-7.47 (m, 1H), 7.38-7.36 (m, 1H), 7.07-7.02 (m, 2H), 6.87-6.86 (m, 2H), 5.09-5.03 (m, 1H), 3.94-3.91 (m, 1H), 3.81-3.75 (m, 1H), 3.67-3.63 (m, 2H), 3.47-3.34 (m, 2H), 3.00-2.93 (m, 1H), 2.71 (s, 3H), 2.54-2.50 (m, 2H), 1.95-1.81 (m, 2H), 1.51-1.47 (m, 1H).

### Synthesis of (R)-3-(5-hydroxypent-1-yn-1-yl)-N-(8-methylisoquinolin-1-yl)-N-(piperidin-3-yl) benzamide (Example 513)

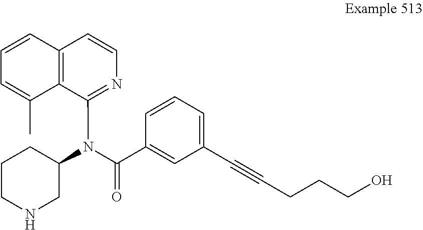 [SMILES: Cc1cccc2ccnc(N(C(=O)c3cccc(C#CCCCO)c3)[C@@H]3CCCNC3)c12]

Example 513 was prepared according to the procedure described for Example 512 by substituting 3-butynol with pent-4-yn-1-ol, provided the title compound hydrochloride salt as a white solid. LC-MS: [M+H⁺]=428.25. ¹H NMR (CD₃OD, 400 MHZ): δ 8.52 (s, 1H), 8.48 (d, J=5.6 Hz, 1H), 7.80 (d, J=5.6 Hz, 1H), 7.69 (d, J=8.0 Hz, 1H), 7.50-7.46 (m, 1H), 7.38-7.36 (m, 1H), 7.03-7.00 (m, 1H), 6.96-6.86 (m, 3H), 5.05-5.00 (m, 1H), 3.91-3.87 (m, 1H), 3.73-3.70 (m, 1H), 3.64 (t, J=6.0 Hz, 2H), 3.34-3.29 (m, 1H), 2.91-2.87 (m, 1H), 2.71 (s, 3H), 2.41 (t, J=7.2 Hz, 2H), 1.91-1.71 (m, 5H), 1.49-1.45 (m, 1H).

### Synthesis of (R)-3-(6-hydroxyhex-1-yn-1-yl)-N-(8-methylisoquinolin-1-yl)-N-(piperidin-3-yl)benzamide (Example 514)

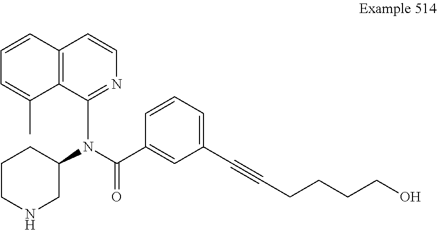 [SMILES: Cc1cccc2ccnc(N(C(=O)c3cccc(C#CCCCCO)c3)[C@@H]3CCCNC3)c12]

Example 514 was prepared according to the procedure described for Example 512 by substituting 3-butynol with pent-4-yn-1-ol, provided the title compound hydrochloride salt as a white solid. LC-MS: [M+H⁺]=442.25. ¹H NMR (CD₃OD, 400 MHZ): δ 8.49 (d, J=5.6 Hz, 1H), 7.81 (d, J=5.6 Hz, 1H), 7.69 (d, J=8.0 Hz, 1H), 7.51-7.47 (m, 1H), 7.38-7.36 (m, 1H), 7.03-7.02 (m, 1H), 6.93-6.86 (m, 3H), 5.09-5.03 (m, 1H), 3.93-3.90 (m, 1H), 3.81-3.79 (m, 1H), 3.60 (t, J=6.4 Hz, 2H), 3.39-3.34 (m, 1H), 3.00-2.96 (m, 1H), 2.71 (s, 3H), 2.35 (t, J=6.4 Hz, 2H), 1.95-1.90 (m, 2H), 1.85-1.81 (m, 1H), 1.65-1.59 (m, 5H).

### Synthesis of (R)-3-(4-aminobut-1-yn-1-yl)-N-(8-methylisoquinolin-1-yl)-N-(piperidin-3-yl)benzamide (Example 515)

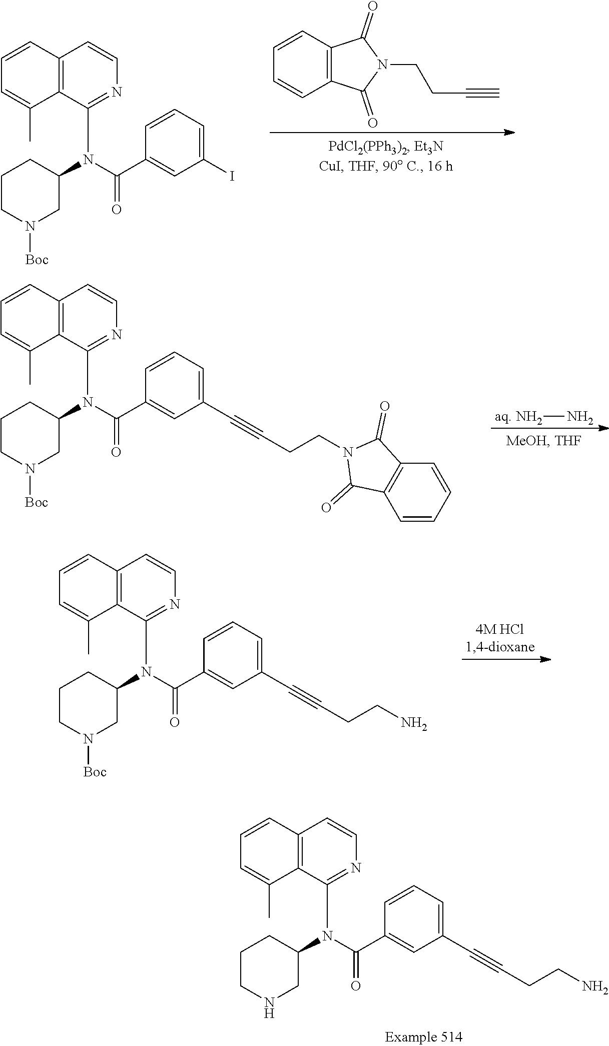

### Step 1:

To a solution of tert-butyl (R)-3-(3-iodo-N-(8-methylisoquinolin-1-yl) benzamido) piperidine-1-carboxylate (50 mg, 0.087 mmol) in THF (1 mL) were added 2-(but-3-yn-1-yl)isoindoline-1,3-dione (34.9 mg, 0.175 mmol), Et₃N (0.024 mL, 0.175 mmol) and CuI (8.33 mg, 0.044 mmol) at room temperature. The reaction mixture was purged with argon for 5 min and followed by addition of PdCl₂(pph₃)₂ (6.14 mg, 8.75 mmol). The reaction mixture was stirred at 90° C. for 16 h before it was diluted with water and extracted with EtOAc (2×20 mL). The combined organic layer was washed with brine, dried over Na₂SO₄ and concentrated. The residue was purified by Combi-flash (30-40% EtOAc in hexane) to afford tert-butyl (R)-3-(3-(4-(1,3-dioxoisoindolin-2-yl)but-1-yn-1-yl)-N-(8-methylisoquinolin-1-yl) benzamido) piperidine-1-carboxylate (40 mg, 56%) as a yellow solid. LC-MS: [M+H⁺]=643.4.

### Step 2:

To a solution of tert-butyl (R)-3-(3-(4-(1,3-dioxoisoindolin-2-yl)but-1-yn-1-yl)-N-(8-methylisoquinolin-1-yl) benzamido) piperidine-1-carboxylate (60 mg, 0.093 mmol) in MeOH (1 mL) and THF (1 mL) was added hydrazine hydrate (0.025 mL, 0.276 mmol) at room temperature. The reaction mixture was stirred at 70° C. for 2 h before it was diluted with water and extracted with EtOAc (2×20 mL). The combined organic layer was washed with brine, dried over Na₂SO₄, and concentrated under reduced pressure to afford tert-butyl (R)-3-(3-(4-aminobut-1-yn-1-yl)-N-(8-methylisoquinolin-1-yl) benzamido) piperidine-1-carboxylate (70 mg). The residue was further purified by Prep-HPLC (Conditions: Kinetex evo C-18 (250×21.2×5 μm), 0.1% formic acidformic acid in water, Mobile phase B: 100% MeCN), FLOW: 15 mL/min, GRADIENT T/% B: 0/50, 10/80, MeCN+THF). Pure fractions were dried by lyophilization to afford tert-butyl (R)-3-(3-(4-aminobut-1-yn-1-yl)-N-(8-methylisoquinolin-1-yl)benzamido)piperidine-1-carboxylate formic acid salt (40 mg, 56.6%) as an off-white solid. LC-MS: [M+H⁺]=513.3.

### Step 3:

The title compound hydrochloride salt was obtained as an off-white solid by deprotection Method A used in Example 97. LC-MS: [M+H⁺]=413.3. ¹H NMR (CD₃OD, 400 MHZ): δ 8.49 (d, J=5.6 Hz, 1H), 7.81 (d, J=5.6 Hz, 1H), 7.70-7.68 (m, 1H), 7.51-7.47 (m, 1H), 7.39-7.36 (m, 1H), 7.19 (s, 1H), 7.17-7.13 (m, 1H), 6.95-6.84 (m, 2H), 5.09-5.03 (m, 1H), 3.92-3.82 (m, 1H), 3.81-3.75 (m, 1H), 3.39-3.34 (m, 1H), 3.13-3.11 (m, 1H), 2.78-2.75 (m, 2H), 2.73 (s, 3H), 1.94-1.89 (m, 2H), 1.84-1.80 (m, 1H), 1.48-1.28 (m, 2H), 0.94-0.91 (m, 1H).

### Synthesis of (R)—N-(8-methylisoquinolin-1-yl)-3-(5-oxo-5-(pyridin-4-ylamino)pent-1-yn-1-yl)-N-(piperidin-3-yl)benzamide (Example 516)

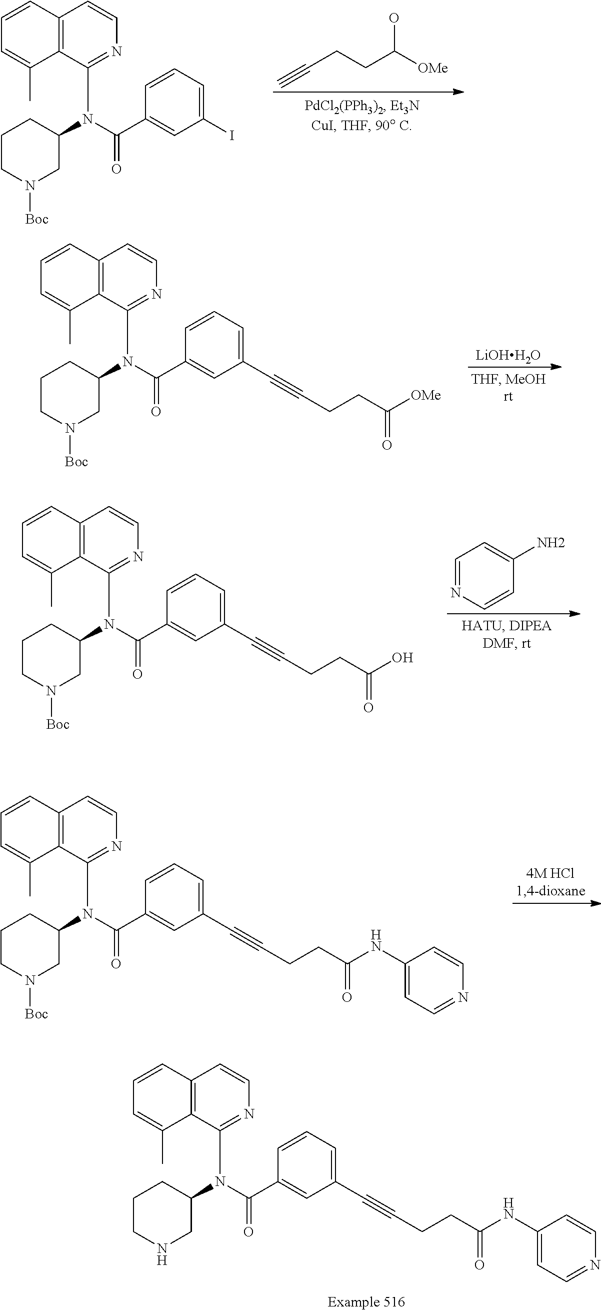

### Steps 1-2:

(R)-5-(3-((1-(tert-butoxycarbonyl)piperidin-3-yl)(8-methylisoquinolin-1-yl) carbamoyl) phenyl) pent-4-ynoic acid was prepared according to the procedure described for Example 589 by substituting 3-butynol with methyl pent-4-ynoate. The resulting ester was hydrolyzed to (R)-5-(3-((1-(tert-butoxycarbonyl)piperidin-3-yl)(8-methylisoquinolin-1-yl) carbamoyl) phenyl) pent-4-ynoic acid with the treatment of aq. LiOH in a mixture of THF and MeOH at 25° C.

### Step 3:

To a solution of (R)-5-(3-((1-(tert-butoxycarbonyl)piperidin-3-yl)(8-methylisoquinolin-1-yl)carbamoyl) phenyl) pent-4-ynoic acid (100 mg, 0.185 mmol) in DMF (2 mL) were added DIPEA (0.096 mL, 0.554 mmol), HATU (211 mg, 0.554 mmol) and pyridin-4-amine (17 mg, 0.185 mmol)) at room temperature and stirred for 16 h. The reaction mixture was diluted with ice cold water (10 mL) and extracted with EtOAc (2×50 mL). The combined organic layer was washed with brine, dried over Na₂SO₄, and concentrated under reduced pressure to afford tert-butyl (R)-3-(N-(8-methylisoquinolin-1-yl)-3-(5-oxo-5-(pyridin-4-ylamino) pent-1-yn-1-yl) benzamido) piperidine-1-carboxylate (100 mg) as an off-white solid. The residue was purified by Prep-HPLC (Conditions: Kinetex evo C-18 (250×21.2×5 μm), Mobile phase A: 0.1% formic acid in water, Mobile phase B: 100% MeCN), FLOW: 15 mL/min, Gradient: T/% B: 0/10, 6/60, 10/25, MeCN+THF). Pure fractions were dried by lyophilization afford tert-butyl (R)-3-(N-(8-methylisoquinolin-1-yl)-3-(5-oxo-5-(pyridin-4-ylamino)pent-1-yn-1-yl) benzamido) piperidine-1-carboxylate (40 mg, 38.1%) as an off-white solid. LC-MS: [M+H⁺]=618.3.

### Step 4:

The title compound hydrochloride salt was obtained as an off-white solid by deprotection Method A used in Example 97. LC-MS: [M+H⁺]=516.2. ¹H NMR (CD₃OD, 400 MHZ): δ 8.61 (d, J=7.2 Hz, 2H),) 8.47 (d, J=5.6 Hz, 1H),) 8.17 (d, J=7.2 Hz, 2H), 7.79 (d, J=5.6 Hz, 1H), 7.71-7.61 (m, 2H), 7.50-7.46 (m, 1H), 7.38-7.35 (m, 1H), 7.03-7.00 (m, 2H), 6.85-6.82 (m, 2H), 5.08-4.96 (m, 1H), 3.94-3.90 (m, 1H), 3.80-3.74 (m, 1H), 3.42-3.36 (m, 1H), 3.13-2.95 (m, 1H), 2.80-2.752 (m, 2H), 2.70 (s, 3H), 1.94-1.92 (m, 2H), 1.84-1.80 (m, 1H), 1.43-1.28 (m, 2H), 0.96-0.89 (m, 1H).

### Synthesis of (R)—N-(8-methylisoquinolin-1-yl)-3-(5-oxo-5-((pyridin-4-ylmethyl)amino)pent-1-yn-1-yl)-N-(piperidin-3-yl)benzamide (Example 517)

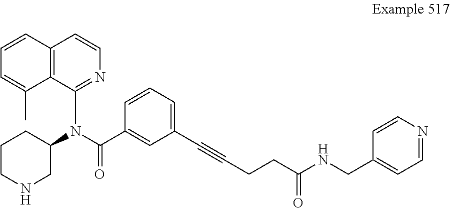 [SMILES: Cc1cccc2ccnc(N(C(=O)c3cccc(C#CCCC(=O)NCc4ccncc4)c3)[C@@H]3CCCNC3)c12]

Example 517 was prepared according to the procedure described for Example 516 by substituting 4-aminopyridine with pyridin-4-ylmethanamine, provided the title compound hydrochloride salt as a white solid. LC-MS: [M+H⁺]=532.3. ¹H NMR (CD₃OD, 400 MHZ): δ 8.58-8.56 (m, 2H), 8.49 (d, J=5.6 Hz, 1H), 7.94 (d, J=6.4 Hz, 2H), 7.80 (d, J=5.6 Hz, 1H), 7.68-7.64 (m, 1H), 7.48-7.44 (m, 1H), 7.39-7.36 (m, 1H), 7.08 (s, 1H), 7.05-7.03 (m, 1H), 6.87-6.85 (m, 2H), 5.07-4.96 (m, 1H), 4.67 (s, 2H), 3.94-3.90 (m, 1H), 3.81-3.75 (m, 1H), 3.39-3.34 (m, 1H), 3.13-2.97 (m, 1H), 2.71-2.58 (m, 5H), 2.58 (t, J=7.2 Hz, 2H), 1.94-1.80 (m, 3H), 1.45-1.28 (m, 1H).

### Synthesis of 3-(5-(3-hydroxypyrrolidin-1-yl)-5-oxopent-1-yn-1-yl)-N-(8-methylisoquinolin-1-yl)-N—((R)-piperidin-3-yl)benzamide (Example 518)

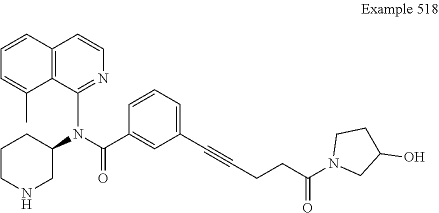 [SMILES: Cc1cccc2ccnc(N(C(=O)c3cccc(C#CCCC(=O)N4CCC(O)C4)c3)[C@@H]3CCCNC3)c12]

Example 518 was prepared according to the procedure described for Example 516 by substituting 4-aminopyridine with pyrrolidin-3-ol, provided the title compound hydrochloride salt as a white solid. LC-MS: [M+H⁺]=511.4. ¹H NMR (CD₃OD, 400 MHZ): δ 8.47-8.46 (m, 1H), 7.78 (d, J=5.6 Hz, 1H), 7.68-7.65 (m, 1H), 7.48-7.38 (m, 2H), 7.04-7.02 (m, 2H), 6.89-6.85 (m, 2H), 4.48-4.38 (m, 1H), 3.82-3.80 (m, 1H), 3.65-3.46 (m, 5H), 3.21-3.13 (m, 1H), 2.82-2.72 (m, 1H), 2.71 (s, 3H), 2.64-2.56 (m, 4H), 2.04-1.97 (m, 2H), 1.84-1.74 (m, 3H), 1.28-1.18 (m, 1H), 0.91-0.87 (m, 1H).

### Synthesis of (R)—N-(8-methylisoquinolin-1-yl)-3-(5-morpholino-5-oxopent-1-yn-1-yl)-N-(piperidin-3-yl)benzamide (Example 519)

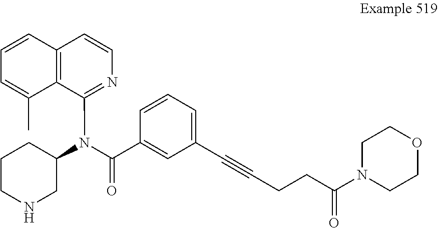 [SMILES: Cc1cccc2ccnc(N(C(=O)c3cccc(C#CCCC(=O)N4CCOCC4)c3)[C@@H]3CCCNC3)c12]

Example 519 was prepared according to the procedure described for Example 516 by substituting 4-aminopyridine with morpholine, provided the title compound hydrochloride salt as a white solid. LC-MS: [M+H⁺]=511.55. ¹H NMR (CD₃OD, 400 MHZ): δ8.49-8.44 (m, 1H), 7.82 (d, J=5.6 Hz, 1H), 7.72-7.61 (m, 1H), 7.52-7.48 (m, 1H), 7.39-7.38 (m, 1H), 7.05-7.03 (m, 2H), 6.89-6.85 (m, 2H), 5.09-4.97 (m, 1H), 4.23-4.19 (m, 1H), 3.96-3.78 (m, 2H), 3.65-3.55 (m, 6H), 3.40-3.34 (m, 1H), 3.22-2.97 (m, 2H), 2.77-2.69 (m, 3H), 2.64 (s, 3H), 1.92-1.66 (m, 3H), 1.39-1.28 (m, 1H), 0.96-0.87 (m, 1H).

### Synthesis of 3-(5-((1-hydroxypropan-2-yl)amino)-5-oxopent-1-yn-1-yl)-N-(8-methylisoquinolin-1-yl)-N—((R)-piperidin-3-yl)benzamide (Example 520)

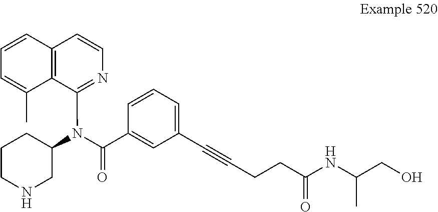 [SMILES: Cc1cccc2ccnc(N(C(=O)c3cccc(C#CCCC(=O)NC(C)CO)c3)[C@@H]3CCCNC3)c12]

Example 520 was prepared according to the procedure described for Example 516 by substituting 4-aminopyridine with 2-aminopropan-1-ol, provided the title compound hydrochloride salt as a white solid. LC-MS: [M+H⁺]=499.3. ¹H NMR (CD₃OD, 400 MHZ): δ 8.49 (d, J=5.2 Hz, 1H), 7.81 (d, J=6.4 Hz, 1H), 7.70-7.66 (m, 1H), 7.51-7.47 (m, 1H), 7.38-7.36 (m, 1H), 7.05-7.02 (m, 2H), 6.86-6.83 (m, 2H), 5.09-5.02 (m, 1H), 3.97-3.91 (m, 2H), 3.81-3.76 (m, 1H), 3.48-3.44 (m, 2H), 2.98-2.96 (m, 1H), 2.76-2.60 (m, 6H), 2.41 (t, J=7.2 Hz, 2H), 1.94-1.80 (m, 3H), 1.51-1.47 (m, 1H), 1.12 (d, J=6 Hz, 3H).

### Synthesis of (R)-2-fluoro-4-(1-methyl-1H-1,2,3-triazol-4-yl)-N-(2-(4-(methylcarbamoyl)phenyl)thieno[3,2-c]pyridin-4-yl)-N-(piperidin-3-yl)benzamide (Example 521)

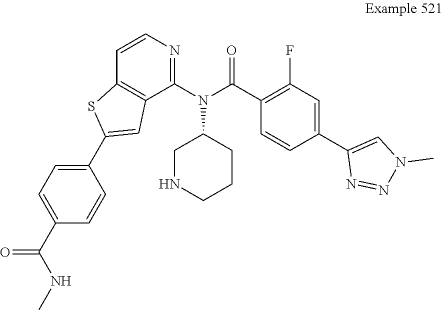 [SMILES: CNC(=O)c1ccc(-c2cc3c(N(C(=O)c4ccc(-c5cn(C)nn5)cc4F)[C@@H]4CCCNC4)nccc3s2)cc1]

Example 521 was prepared according to the procedure described for Example 523 by substituting aniline with methylamine, provided the title compound (28%) as a white solid. LC-MS: [M+H⁺]=570.3. ¹H NMR (400 MHZ, DMSO-d₆): δ 9.08 (d, J=3.8 Hz, 2H), 8.63-8.53 (m, 1H), 8.47-8.22 (m, 2H), 8.19-8.08 (m, 1H), 8.05-7.91 (m, 5H), 7.44-7.19 (m, 3H), 5.24-4.81 (m, 1H), 4.00 (s, 3H), 3.82-3.70 (m, 1H), 3.42-3.30 (m, 1H), 3.25-3.13 (m, 1H), 2.83 (d, J=4.4 Hz, 3H), 2.77-2.69 (m, 1H), 2.28-2.03 (m, 1H), 1.94-1.71 (m, 2H), 1.28-1.03 (m, 1H).

### Synthesis of 2-fluoro-N-[2-[4-(isopropylcarbamoyl)phenyl]thieno[3,2-c]pyridin-4-yl]-4-(1-methyltriazol-4-yl)-N-[(3R)-3-piperidyl]benzamide (Example 522)

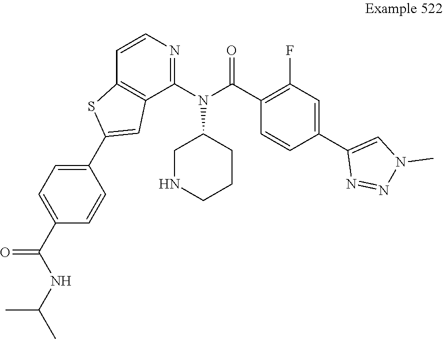 [SMILES: CC(C)NC(=O)c1ccc(-c2cc3c(N(C(=O)c4ccc(-c5cn(C)nn5)cc4F)[C@@H]4CCCNC4)nccc3s2)cc1]

Example 522 was prepared according to the procedure described for Example 523 by substituting aniline with propan-2-amine, provided the title compound hydrochloride salt as a white solid. LC-MS: [M+H⁺]=598.4. ¹H NMR (400 MHZ, DMSO-d₆): δ 9.34-8.92 (m, 2H), 8.42 (s, 1H), 8.39-8.32 (m, 2H), 8.11 (s, 1H), 8.03-7.93 (m, 5H), 7.41-7.18 (m, 3H), 5.19-5.05 (m, 1H), 4.16 (br d, J=6.6 Hz, 1H), 3.99 (s, 3H), 3.78-3.67 (m, 1H), 3.37 (q, J=10.3 Hz, 1H), 3.19 (br d, J=9.9 Hz, 1H), 2.78-2.69 (m, 1H), 2.06 (br d, J=13.0 Hz, 1H), 1.96-1.69 (m, 3H), 1.20 (d, J=6.6 Hz, 7H).

### Synthesis of 2-fluoro-4-(1-methyltriazol-4-yl)-N-[2-[4-(phenylcarbamoyl)phenyl] thieno[3,2-c]pyridin-4-yl]-N-[(3R)-3-piperidyl]benzamide (Example 523)

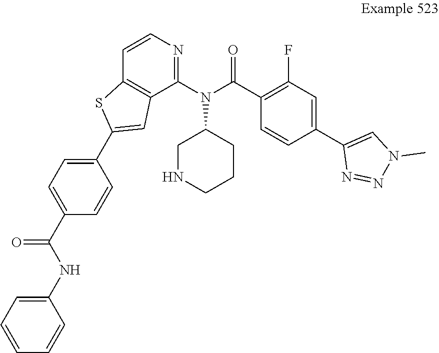 [SMILES: Cn1cc(-c2ccc(C(=O)N(c3nccc4sc(-c5ccc(C(=O)Nc6ccccc6)cc5)cc34)[C@@H]3CCCNC3)c(F)c2)nn1]

Example 523 was prepared according to the procedure described for Example 540 by substituting 3-[4-[[(3R)-1-tert-butoxycarbonyl-3-piperidyl]-[2-fluoro-4-(1-methyltriazol-4-yl)benzoyl]amino]thieno[3,2-c]pyridin-2-yl]benzoic acid with 4-[4-[[(3R)-1-tert-butoxycarbonyl-3-piperidyl]-[2-fluoro-4-(1-methyltriazol-4-yl)benzoyl]amino]thieno[3,2-c]pyridin-2-yl]benzoic acid, provided the title compound hydrochloride salt as a light yellow solid. LC-MS: [M+H]⁺=632.4. ¹H NMR (400 MHZ, DMSO-d6): δ 10.36 (s, 1H), 8.98-8.77 (m, 2H), 8.45-8.33 (m, 2H), 8.18-8.08 (m, 3H), 8.07-7.99 (m, 3H), 7.81 (d, J=7.8 Hz, 2H), 7.41-7.29 (m, 4H), 7.27-7.20 (m, 1H), 7.17-7.09 (m, 1H), 5.20-4.92 (m, 1H), 3.99 (s, 3H), 3.77-3.68 (m, 1H), 3.36-3.35 (m, 2H), 3.25-3.17 (m, 1H), 2.79-2.69 (m, 1H), 2.11-1.68 (m, 3H), 1.24-1.08 (m, 1H).

### Synthesis of N-[2-(3-aminophenyl)thieno[3,2-c]pyridin-4-yl]-2-fluoro-4-(1-methyltriazol-4-yl)-N-[(3R)-3-piperidyl]benzamidezamide (Example 524)

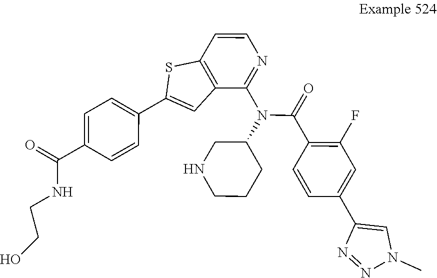 [SMILES: Cn1cc(-c2ccc(C(=O)N(c3nccc4sc(-c5ccc(C(=O)NCCO)cc5)cc34)[C@@H]3CCCNC3)c(F)c2)nn1]

Example 524 was prepared according to the procedure described for Example 413 by substituting 2-fluoro-4-(triazolo[4,5-b]pyridin-3-yl)benzoyl chloride with 2-fluoro-4-(1-methyltriazol-4-yl)benzoyl chloride, providing the title compound as an off-white solid. LC-MS: [M+H⁺]=600.5. ¹H NMR (400 MHZ, DMSO-d₆): δ 9.36-9.19 (m, 1H), 9.16-8.86 (m, 1H), 8.69-8.55 (m, 1H), 8.51-8.30 (m, 2H), 8.14 (s, 1H), 8.08-7.90 (m, 5H), 7.49-7.04 (m, 3H), 5.21-4.92 (m, 1H), 3.99 (s, 3H), 3.71 (br s, 1H), 3.59-3.52 (m, 2H), 3.44-3.29 (m, 3H), 3.26-3.13 (m, 1H), 2.81-2.62 (m, 1H), 2.30-2.01 (m, 1H), 1.97-1.71 (m, 2H), 1.25-1.03 (m, 1H)

### Synthesis of 2-fluoro-N-[2-[(E)-3-(methylamino)-3-oxo-prop-1-enyl]thieno[3,2-c]pyridin-4-yl]-N-[(3R)-3-piperidyl]-4-(triazolo[4,5-b]pyridin-3-yl)benzamide (Example 525)

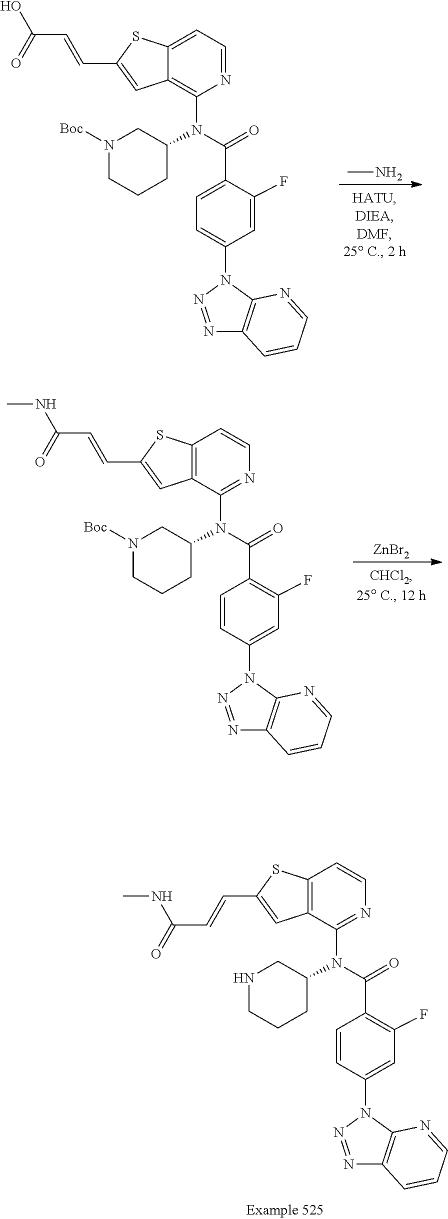

### Step 1:

To a solution of (E)-3-[4-[[(3R)-1-tert-butoxycarbonyl-3-piperidyl]-[2-fluoro-4-(triazolo[4,5-b]pyridin-3-yl)benzoyl]amino]thieno[3,2-c]pyridin-2-yl]prop-2-enoic acid (50 mg, 77.68 μmol) in dimethylformamide (2 mL) were added EDCI (29.78 mg, 155.36 μmol) and HOBt (20.99 mg, 155.36 μmol) at 25° C. The mixture was stirred at 25° C. for 10 min. Then methanamine hydrochloride (10.49 mg, 155.36 μmol) and DIEA (20.08 mg, 155.36 μmol) were added to the mixture at 25° C. The mixture was stirred at 25° C. for 2 h. The reaction mixture was filtered and the filtrate was concentrated. The residue was purified by Prep-HPLC (column: Waters Xbridge Prep OBD C18 150*40 mm*10 μm; mobile phase: [H₂O (10 mM NH₄HCO₃)-MeCN]; gradient: 30%-70% B over 8.0 min) to give tert-butyl (3R)-3-[[2-fluoro-4-(triazolo[4,5-b]pyridin-3-yl)benzoyl]-[2-[(E)-3-(methylamino)-3-oxo-prop-1-enyl]thieno[3,2-c]pyridin-4-yl]amino]piperidine-1-carboxylate (40 mg, 78%) as a yellow solid.

### Step 2:

To a solution of tert-butyl (3R)-3-[[2-fluoro-4-(triazolo[4,5-b]pyridin-3-yl)benzoyl]-[2-[(E)-3-(methylamino)-3-oxo-prop-1-enyl]thieno[3,2-c]pyridin-4-yl]amino]piperidine-1-carboxylate (40 mg, 60.91 μmol) in dichloromethane (4 mL) was added ZnBr₂ (137.16 mg, 609.08 μmol). The mixture was stirred at 25° C. for 12 h before it was concentrated. The residue was purified by prep-HPLC (column: Waters Xbridge Prep OBD C18 150*40 mm*10 μm; mobile phase: [H₂O (10 mM NH₄HCO₃)-MeCN]; gradient: 10%-60% B over 8.0 min) to afford 2-fluoro-N-[2-[(E)-3-(methylamino)-3-oxo-prop-1-enyl]thieno[3,2-c]pyridin-4-yl]-N-[(3R)-3-piperidyl]-4-(triazolo[4,5-b]pyridin-3-yl)benzamide (13.4 mg, 39%) as an off-white solid. LC-MS: [M+H⁺]=556.6. ¹H NMR (400 MHZ, CD₃OD): δ 8.77 (br s, 1H), 8.51 (br d, J=8.6 Hz, 1H), 8.40-8.31 (m, 1H), 8.14-8.04 (m, 1H), 8.03-7.94 (m, 1H), 7.86-7.75 (m, 2H), 7.72 (s, 1H), 7.59-7.50 (m, 1H), 7.48-7.35 (m, 1H), 6.56-6.44 (m, 1H), 4.64-4.53 (m, 1H), 3.63-3.51 (m, 1H), 3.00-2.91 (m, 1H), 2.86 (s, 3H), 2.45-2.33 (m, 1H), 2.09-1.93 (m, 1H), 1.81-1.60 (m, 2H), 1.37-1.23 (m, 1H).

### Synthesis of N-[2-[(E)-3-(dimethylamino)-3-oxo-prop-1-enyl]thieno[3,2-c]pyridin-4-yl]-2-fluoro-N-[(3R)-3-piperidyl]-4-(triazolo[4,5-b]pyridin-3-yl)benzamide (Example 526)

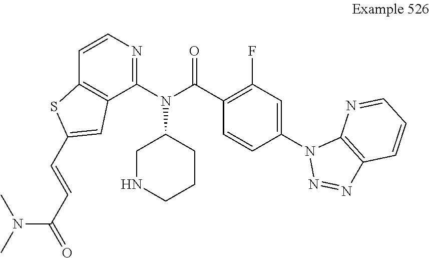 [SMILES: CN(C)C(=O)/C=C/c1cc2c(N(C(=O)c3ccc(-n4nnc5cccnc54)cc3F)[C@@H]3CCCNC3)nccc2s1]

Example 526 was prepared according to the procedure described for Example 525, by substituting methanamine hydrochloride with N-methylmethanamine, providing the title compound as a yellow solid. LC-MS: [M+H⁺]=571.4. ¹H NMR (400 MHZ, CD₃OD): δ 8.77 (br s, 1H), 8.51 (br d, J=8.3 Hz, 1H), 8.36 (br d, J=5.1 Hz, 1H), 8.13-8.03 (m, 1H), 8.02-7.95 (m, 1H), 7.86-7.73 (m, 3H), 7.54 (br dd, J=4.0, 7.0 Hz, 1H), 7.47-7.35 (m, 1H), 7.00 (br d, J=15.1 Hz, 1H), 4.82-4.48 (m, 1H), 3.62-3.47 (m, 1H), 3.21 (s, 3H), 3.07 (s, 3H), 3.01-2.89 (m, 1H), 2.47-2.28 (m, 2H), 2.10-1.91 (m, 1H), 1.87-1.61 (m, 2H), 1.40-1.19 (m, 1H).

### Synthesis of 4-[4-[[2-fluoro-4-(triazolo[4,5-b]pyridin-3-yl)benzoyl]-[(3R)-3-piperidyl]amino]thieno[3,2-c]pyridin-2-yl]-N-methyl-thiazole-2-carboxamide (Example 527)

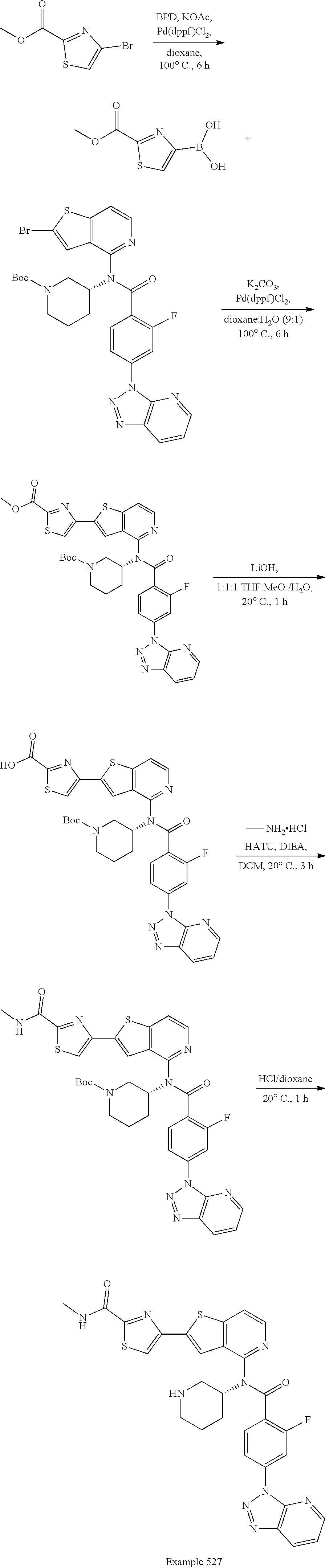

### Step 1:

To a solution of methyl 4-bromothiazole-2-carboxylate (150 mg, 675.49 μmol) in dioxane (5 mL) were added BPD (343.07 mg, 1.35 mmol), Pd(dppf)Cl₂ (24.71 mg, 33.77 μmol) and KOAc (132.59 mg, 1.35 mmol) at 20° C. under N₂. The reaction mixture was stirred at 100° C. for 6 hours. The reaction mixture was poured into water (20 mL) and extracted with ethyl acetate (3×20 mL). The combined organic phases were washed with brine (100 mL), dried with anhydrous Na₂SO₄, and concentrated in vacuum to give (2-methoxycarbonylthiazol-4-yl)boronic acid (150 mg, 59%) as a light yellow solid, which was used directly in the next step.

### Step 2:

To a solution of tert-butyl (3R)-3-[(2-bromothieno[3,2-c]pyridin-4-yl)-[2-fluoro-4-(triazolo[4,5-b]pyridin-3-yl)benzoyl]amino]piperidine-1-carboxylate (100 mg, 137.92 μmol) and (2-methoxycarbonylthiazol-4-yl)boronic acid (128.95 mg, 344.81 μmol) in dioxane (5 mL) and H₂O (0.5 mL) were added K₂CO₃ (57.19 mg, 413.77 μmol) and Pd(dppf)Cl₂ (10.09 mg, 13.79 μmol) at 20° C. under N₂. The reaction mixture was stirred at 100° C. for 6 hours The reaction mixture was filtered, and the filtrate was concentrated to give methyl 4-[4-[[(3R)-1-tert-butoxycarbonyl-3-piperidyl]-[2-fluoro-4-(triazolo[4,5-b]pyridin-3-yl)benzoyl]amino]thieno[3,2-c]pyridin-2-yl]thiazole-2-carboxylate (150 mg, 76%) as a brown oil, which was used directly in the next step.

### Step 3:

To a solution of methyl 4-[4-[[(3R)-1-tert-butoxycarbonyl-3-piperidyl]-[2-fluoro-4-(triazolo[4,5-b]pyridin-3-yl)benzoyl]amino]thieno[3,2-c]pyridin-2-yl]thiazole-2-carboxylate (150 mg, 104.93 μmol) in THF (5 mL), MeOH (5 mL), and H₂O (5 mL) was added LiOH (5.03 mg, 209.85 μmol) at 20° C. The reaction mixture was stirred at 20° C. for 1 h before it was concentrated. The residue was purified by Prep-HPLC (column: Phenomenex Luna C18 100*40 mm*5 μm; mobile phase: [H₂O (0.04% HCl)-MeCN]; gradient: 35%-75% B over 8.0 min]; gradient: 35%-75% B over 8.0 min). The product was lyophilized to give 4-[4-[[(3R)-1-tert-butoxycarbonyl-3-piperidyl]-[2-fluoro-4-(triazolo[4,5-b]pyridin-3-yl)benzoyl]amino]thieno[3,2-c]pyridin-2-yl]thiazole-2-carboxylic acid (45 mg, 44%) as an off-white solid.

### Step 4:

To a solution of 4-[4-[[(3R)-1-tert-butoxycarbonyl-3-piperidyl]-[2-fluoro-4-(triazolo[4,5-b]pyridin-3-yl)benzoyl]amino]thieno[3,2-c]pyridin-2-yl]thiazole-2-carboxylic acid (20 mg, 27.11 μmol) and methanamine; hydrochloride (3.66 mg, 54.23 μmol) in DCM (2 mL) at 20° C. under N₂ atmosphere was added DIEA (10.51 mg, 81.34 μmol) and then HATU (30.93 mg, 81.34 μmol). The reaction mixture was stirred at 20° C. for 12 h. The reaction mixture was poured into water (20 mL), and extracted with ethyl acetate (3×20 mL). The combined organic layer was washed with brine (50 mL), dried with anhydrous Na₂SO₄, and concentrated. The residue was purified by Prep-HPLC (column: Phenomenex Luna C18 100*40 mm*5 μm; mobile phase: [H₂O (0.04% HCl)-MeCN]; gradient: 50%-75% B over 8.0 min). The product was lyophilized to give tert-butyl (3R)-3-[[2-fluoro-4-(triazolo[4,5-b]pyridin-3-yl)benzoyl]-[2-[2-(methylcarbamoyl)thiazol-4-yl]thieno[3,2-c]pyridin-4-yl]amino]piperidine-1-carboxylate (9 mg, 44%) as an off-white solid.

### Step 5:

The title compound hydrochloride salt was obtained as an off-white solid by deprotection Method A used in Example 97. LC-MS: [M+H⁺]=614.4. ¹H NMR (400 MHZ, CD₃OD): δ 8.74 (br d, J=4.1 Hz, 1H), 8.57-8.31 (m, 3H), 8.13-7.83 (m, 4H), 7.61-7.39 (m, 2H), 5.39-5.15 (m, 1H), 3.93 (br d, J=11.8 Hz, 1H), 3.67 (br t, J=12.1 Hz, 1H), 3.38 (br s, 1H), 2.98 (s, 3H), 2.92-2.79 (m, 1H), 2.22-1.84 (m, 3H), 1.51-1.35 (m, 1H).

### Synthesis of (R)—N-(2-(4-(dimethylcarbamoyl)phenyl)thieno[3,2-c]pyridin-4-yl)-2-fluoro-4-(1-methyl-1H-1,2,3-triazol-4-yl)-N-(piperidin-3-yl)benzamide (Example 528)

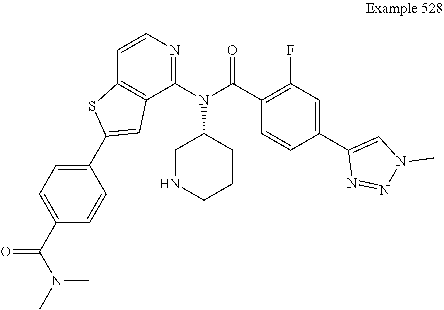 [SMILES: CN(C)C(=O)c1ccc(-c2cc3c(N(C(=O)c4ccc(-c5cn(C)nn5)cc4F)[C@@H]4CCCNC4)nccc3s2)cc1]

Example 528 was prepared according to the procedure described for Example 363 by substituting 4-pyridylboronic acid with (4-(dimethylcarbamoyl)phenyl) boronic acid, providing the title compound hydrochloride salt as an off-white solid. LC-MS: [M+H⁺]=584.4. ¹H NMR (400 MHZ, DMSO-d₆): δ 9.27-8.87 (m, 1H), 8.42 (s, 1H), 8.39-8.33 (m, 1H), 8.11-7.87 (m 4H), 765-7.55 (m, 2H), 7.46-7.21 (m, 3H), 5.15-4.94 (m, 1H), 3.99 (s, 3H), 3.38-3.10 (m, 2H), 3.01-2.96 (m, 6H), 2.70-2.67 (m, 1H), 2.10-1.74 (m, 3H), 1.23-1.13 (m, 1H).

### Synthesis of 2-fluoro-4-(1-methyltriazol-4-yl)-N-[(3R)-3-piperidyl]-N-[2-[4-(1H-pyrazol-3-yl) phenyl] thieno [3, 2-c] pyridin-4-yl] benzamide (Example 529)

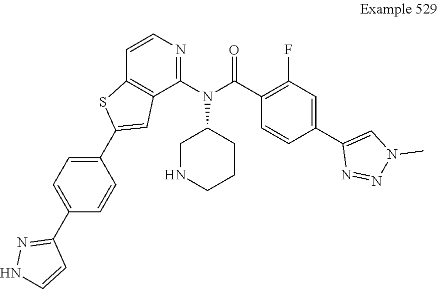 [SMILES: Cn1cc(-c2ccc(C(=O)N(c3nccc4sc(-c5ccc(-c6cc[nH]n6)cc5)cc34)[C@@H]3CCCNC3)c(F)c2)nn1]

Example 528 was prepared according to the procedure described for Example 363 by substituting 4-pyridylboronic acid with [5-(1H-pyrazol-3-yl)-2-pyridyl]boronic acid, providing the title compound as a yellow solid. LC-MS: [M+H⁺]=579.4. ¹H NMR (400 MHz, DMSO-d₆): δ 9.28 (br d, J=11.1 Hz, 1H), 9.17-8.88 (m, 1H), 8.52-8.39 (m, 1H), 8.38-8.28 (m, 1H), 8.09-7.85 (m, 5H), 7.84-7.72 (m, 1H), 7.45-7.17 (m, 2H), 6.92-6.68 (m, 1H), 5.18-4.95 (m, 1H), 3.99 (s, 3H), 3.78-3.64 (m, 1H), 3.46-3.09 (m, 2H), 2.85-2.63 (m, 1H), 2.29-2.01 (m, 1H), 1.99-1.68 (m, 2H), 1.34-1.01 (m, 1H).

### Synthesis of (R)-4-(3H-[1,2,3]triazolo[4,5-b]pyridin-3-yl)-2-fluoro-N-(6-(5-(hydroxymethyl)-1H-1,2,3-triazol-4-yl)isoquinolin-1-yl)-N-(piperidin-3-yl)benzamide (Example 530)

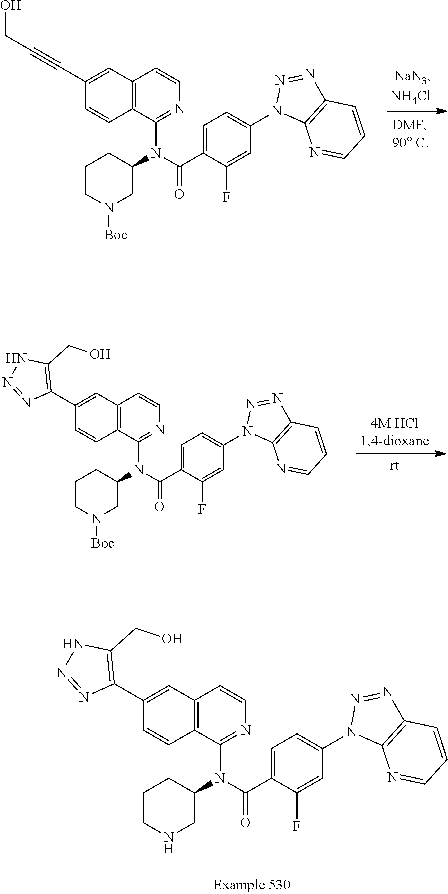 [SMILES: CC(C)(C)OC(=O)N1CCC[C@@H](N(C(=O)c2ccc(-n3nnc4cccnc43)cc2F)c2nccc3cc(C#CCO)ccc23)C1 ; CC(C)(C)OC(=O)N1CCC[C@@H](N(C(=O)c2ccc(-n3nnc4cccnc43)cc2F)c2nccc3cc(-c4nn[nH]c4CO)ccc23)C1 ; O=C(c1ccc(-n2nnc3cccnc32)cc1F)N(c1nccc2cc(-c3nn[nH]c3CO)ccc12)[C@@H]1CCCNC1]

### Step 1:

To a solution of tert-butyl (R)-3-(4-(3H-[1,2,3]triazolo[4,5-b]pyridin-3-yl)-2-fluoro-N-(6-(3-hydroxyprop-1-yn-1-yl)isoquinolin-1-yl)benzamido)piperidine-1-carboxylate (60 mg, 0.097 mmol) in DMF (2 mL) was added ammonium chloride (10.32 mg, 0.193 mmol) and sodium azide (12.55 mg, 0.193 mmol) at room temperature and then the reaction mixture was stirred at 90° C. for 16 h. The reaction mixture was diluted with water (30 mL) and extracted with EtOAc (2×30 mL). The combined organic layer was washed with brine, dried over Na₂SO₄, and concentrated under reduced pressure and was purified by Prep-HPLC (Luna OMEGA C-18 (250×21.2×5 μm), Mobile phase A: 0.1% formic acid in water, Mobile phase B: MeCN (100%), Gradient: Time (min)/% B: 0/10, 10/60, Flow: 15 mL/min, Dilution: MeCN+H₂O). Pure fractions were combined and dried by lyophilization to afford tert-butyl (R)-3-(4-(3H-[1,2,3]triazolo[4,5-b]pyridin-3-yl)-2-fluoro-N-(6-(5-(hydroxymethyl)-1H-1,2,3-triazol-4-yl)isoquinolin-1-yl)benzamido)piperidine-1-carboxylate (12 mg) as an off-white solid. LC-MS: [M+H⁺]=665.3.

### Step 2:

The title compound hydrochloride salt was obtained as an off-white solid by deprotection Method A used in Example 97. LC-MS: [M+H⁺]=565.25.

¹H NMR (CD₃OD, 400 MHZ): δ 8.72-8.71 (m, 1H), 8.50-8.46 (m, 2H), 8.28-8.27 (m, 3H), 8.08-8.02 (m, 1H), 7.91-7.94 (m, 1H), 7.85-7.83 (m, 1H), 7.52-7.48 (m, 1H), 7.38-7.34 (m, 1H), 5.29-5.21 (m, 1H), 4.83 (s, 2H), 4.00-3.97 (m, 1H), 3.74-3.68 (m, 1H), 2.91-2.69 (m, 2H), 2.08-1.91 (m, 3H), 1.33-1.28 (m, 1H).

### Synthesis of (R)-2-fluoro-4-(1-methyl-1H-1,2,3-triazol-4-yl)-N-(2-(2-(methylamino)pyrimidin-4-yl)thieno[3,2-c]pyridin-4-yl)-N-(piperidin-3-yl)benzamide (Example 531)

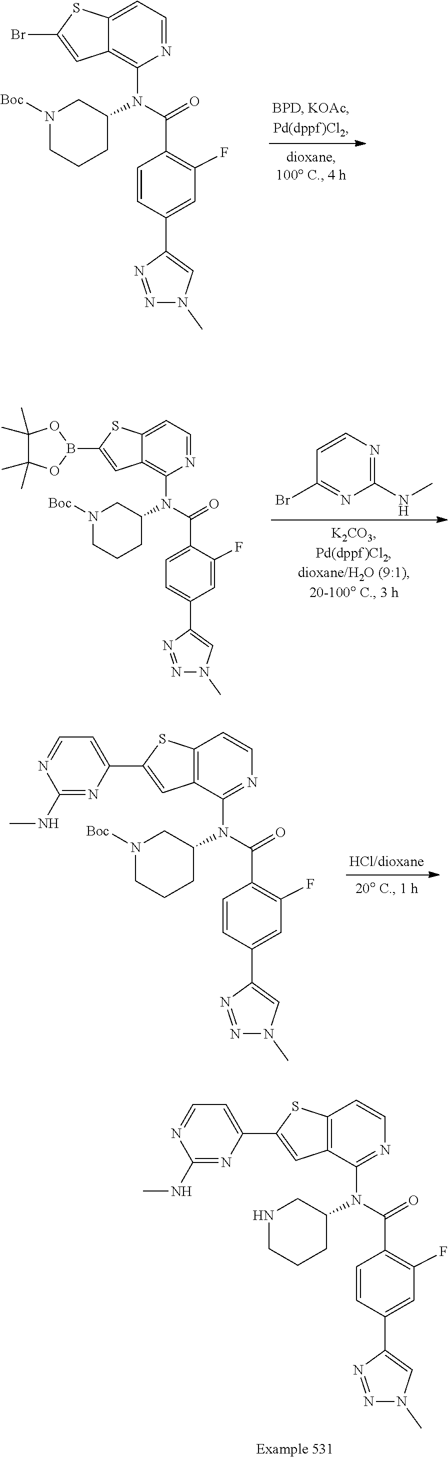

### Step 1:

To a solution of tert-butyl (3R)-3-[(2-bromothieno[3,2-c]pyridin-4-yl)-[2-fluoro-4-(1-methyltriazol-4-yl)benzoyl]amino]piperidine-1-carboxylate (0.2 g, 324.93 μmol) in dioxane (2 mL) were added KOAc (63.78 mg, 649.86 μmol), BPD (247.54 mg, 974.79 μmol), and Pd(dppf)Cl₂ (23.78 mg, 32.49 μmol) at 20° C. The mixture was stirred at 80° C. for 4 h under N₂ Atmosphere. The reaction mixture was filtered. The filtrate was partitioned between EtOAc (50 mL) and water (20 mL). The organic layer was dried over Na₂SO₄, filtered, and concentrated to provide crude tert-butyl (R)-3-(2-fluoro-4-(1-methyl-1H-1,2,3-triazol-4-yl)-N-(2-(4,4,5,5-tetramethyl-1,3,2-dioxaborolan-2-yl)thieno[3,2-c]pyridin-4-yl)benzamido)piperidine-1-carboxylate which was used in the next step directly.

### Step 2:

To a solution of 4-bromo-N-methyl-pyrimidin-2-amine (58.31 mg, 310.11 μmol) in dioxane (1.62 mL) and H₂O (0.18 mL) were added crude tert-butyl (R)-3-(2-fluoro-4-(1-methyl-1H-1,2,3-triazol-4-yl)-N-(2-(4,4,5,5-tetramethyl-1,3,2-dioxaborolan-2-yl)thieno[3,2-c]pyridin-4-yl)benzamido)piperidine-1-carboxylate (0.18 g, 310.11 μmol), K₂CO₃ (128.58 mg, 930.33 μmol), and Pd(dppf)Cl₂ (22.69 mg, 31.01 μmol) at 20° C. under N₂ atmosphere. The mixture was stirred at 100° C. for 1 h. The reaction mixture was filtered, and the filtrate was diluted with water (5 mL) and extracted with ethyl acetate (3×5 mL). The combined organic layer was washed with brine (5 mL), dried over Na₂SO₄, filtered, and concentrated to give crude tert-butyl (3R)-3-[[2-fluoro-4-(1-methyltriazol-4-yl)benzoyl]-[2-[2-(methylamino)pyrimidin-4-yl]thieno[3,2-c]pyridin-4-yl]amino]piperidine-1-carboxylate (0.13 g) as an oil which was used in the next step directly.

### Step 3:

The title compound hydrochloride salt was obtained as a white solid by deprotection Method A used in Example 97. LC-MS: [M+H]=544.2. ¹H NMR (400 MHZ, DMSO-d₆): δ 8.50-8.26 (m, 4H), 8.04-7.81 (m, 1H), 7.55-7.10 (m, 5H), 4.82-4.54 (m, 1H), 4.14-3.92 (m, 4H), 2.99-2.75 (m, 5H), 2.20 (br d, J=4.8 Hz, 1H), 1.98-1.48 (m, 3H), 1.26-0.93 (m, 1H).

### Synthesis of 2-fluoro-4-(1-methyltriazol-4-yl)-N-[(3R)-3-piperidyl]-N-[2-[3-(prop-2-enoylamino)phenyl]thieno[3,2-c]pyridin-4-yl]benzamide (Example 532)

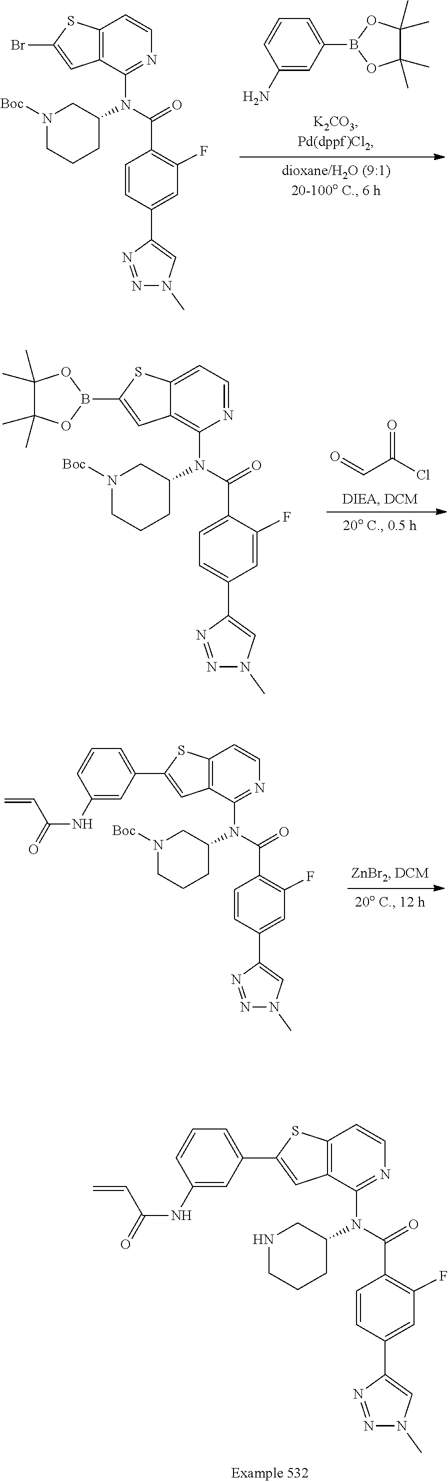 [SMILES: Cn1cc(-c2ccc(C(=O)N(c3nccc4sc(Br)cc34)[C@@H]3CCCN(C(=O)OC(C)(C)C)C3)c(F)c2)nn1 ; CC1(C)OB(c2cccc(N)c2)OC1(C)C ; Cn1cc(-c2ccc(C(=O)N(c3nccc4sc(B5OC(C)(C)C(C)(C)O5)cc34)[C@@H]3CCCN(C(=O)OC(C)(C)C)C3)c(F)c2)nn1 ; O=CC(=O)Cl ; C=CC(=O)Nc1cccc(-c2cc3c(N(C(=O)c4ccc(-c5cn(C)nn5)cc4F)[C@@H]4CCCN(C(=O)OC(C)(C)C)C4)nccc3s2)c1 ; C=CC(=O)Nc1cccc(-c2cc3c(N(C(=O)c4ccc(-c5cn(C)nn5)cc4F)[C@@H]4CCCNC4)nccc3s2)c1]

### Step 1:

To a solution of tert-butyl (3R)-3-[(2-bromothieno[3,2-c]pyridin-4-yl)-[2-fluoro-4-(1-methyltriazol-4-yl)benzoyl]amino]piperidine-1-carboxylate (100 mg, 146.22 μmol) and 3-(4,4,5,5-tetramethyl-1,3,2-dioxaborolan-2-yl) aniline (96.10 mg, 438.66 μmol) in dioxane (3.6 mL) and H₂O (0.4 mL) were added K₂CO₃ (60.63 mg, 438.66 μmol) and Pd(dppf)Cl₂ (10.70 mg, 14.62 μmol) at 20° C. under N₂ atmosphere. The reaction mixture was stirred at 100° C. for 6 h. The reaction mixture was poured into water (20 mL) and extracted with ethyl acetate (3×20 mL). The combined organic phase was washed with brine (100 mL), dried with anhydrous Na₂SO₄, and concentrated to give tert-butyl (3R)-3-[[2-(3-aminophenyl)thieno[3,2-c]pyridin-4-yl]-[2-fluoro-4-(1-methyltriazol-4-yl)benzoyl]amino]piperidine-1-carboxylate (150 mg, 81%) as na oil. LC-MS: [M+H⁺]=628.3

### Step 2:

To a solution of tert-butyl (3R)-3-[[2-(3-aminophenyl)thieno[3,2-c]pyridin-4-yl]-[2-fluoro-4-(1-methyltriazol-4-yl)benzoyl]amino]piperidine-1-carboxylate (180 mg, 143.37 μmol) and prop-2-enoyl chloride (38.93 mg, 430.12 μmol) in THF (4 mL) at 20° C. under N₂ atmosphere was added DIEA (55.59 mg, 430.12 μmol). The reaction mixture was stirred at 20° C. for 0.5 h. The reaction mixture was poured into water (10 mL) and extracted with ethyl acetate (3×20 mL). The combined organic phase was washed with brine (50 mL), dried with anhydrous Na₂SO₄, and concentrated. The residue was purified by prep-HPLC (neutral condition, column: Waters Xbridge Prep OBD C18 150*40 mm*10 μm; mobile phase: [H₂O (10 mM NH₄HCO₃)-MeCN]; gradient: 45%-75% B over 6.0 min). The pure fraction was concentrated to give tert-butyl (3R)-3-[[2-fluoro-4-(1-methyltriazol-4-yl)benzoyl]-[2-[3-(prop-2-enoylamino)phenyl]thieno[3,2-c]pyridin-4-yl]amino]piperidine-1-carboxylate (60 mg, 49%) as a light yellow oil. LC-MS: [M+H⁺]=682.3 Step 3:

To a solution of tert-butyl (3R)-3-[[2-fluoro-4-(1-methyltriazol-4-yl)benzoyl]-[2-[3-(prop-2-enoylamino)phenyl]thieno[3,2-c]pyridin-4-yl]amino]piperidine-1-carboxylate (40 mg, 52.80 μmol) in DCM (5 mL) at 20° C. under N₂ atmosphere was added ZnBr₂ (59.46 mg, 264.02 μmol). The reaction mixture was stirred at 20° C. for 1 h before it was concentrated. The residue was purified by prep-HPLC (neutral condition, column: Waters Xbridge Prep OBD C18 150*40 mm*10 μm; mobile phase: [H₂O (10 mM NH₄HCO₃)-MeCN]; gradient: 20%-50% B over 8.0 min). The pure fraction was lyophilized to give 2-fluoro-4-(1-methyltriazol-4-yl)-N-[(3R)-3-piperidyl]-N-[2-[3-(prop-2-enoylamino)phenyl]thieno[3,2-c]pyridin-4-yl]benzamide (8.5 mg, 19%) as an off-white solid. LC-MS: [M+H⁺]=582.4. ¹H NMR (400 MHz, DMSO-d₆): δ 10.36 (br s, 1H), 8.47-8.27 (m, 2H), 8.14 (br s, 1H), 7.93 (br d, J=4.4 Hz, 1H), 7.83-7.69 (m, 2H), 7.59 (br d, J=7.5 Hz, 1H), 7.49 (br t, J=7.4 Hz, 1H), 7.40-7.25 (m, 2H), 7.16 (br s, 1H), 6.54-6.41 (m, 1H), 6.37-6.26 (m, 1H), 5.81 (dd, J=1.8, 10.1 Hz, 1H), 4.86-4.53 (m, 1H), 3.99 (br s, 3H), 3.67-3.57 (m, 1H), 3.19 (br s, 1H), 2.92-2.73 (m, 1H), 2.69-2.58 (m, 1H), 2.27-2.08 (m, 1H), 1.95-1.41 (m, 3H), 1.18-0.79 (m, 1H).

### Synthesis of 2-fluoro-4-(1-methyltriazol-4-yl)-N-[2-(3-oxoisoindolin-5-yl) thieno[3,2-c]pyridin-4-yl]-N-[(3R)-3-piperidyl]benzamide (Example 533)

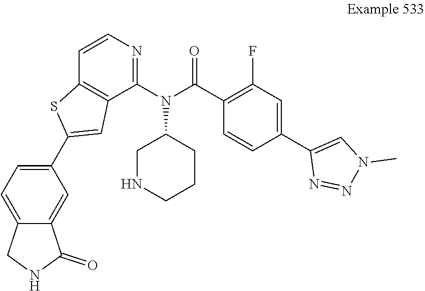 [SMILES: Cn1cc(-c2ccc(C(=O)N(c3nccc4sc(-c5ccc6c(c5)C(=O)NC6)cc34)[C@@H]3CCCNC3)c(F)c2)nn1]

Example 533 was prepared according to the procedure described for Example 363 by substituting 4-pyridylboronic acid with 6-(4,4,5,5-tetramethyl-1,3,2-dioxaborolan-2-yl) isoindolin-1-one, provided the title compound hydrochloride salt as a white solid. LC-MS: [M+H]⁺=568.3. ¹H NMR (400 MHZ, DMSO-d₆): δ 9.05-8.80 (m, 2H), 8.74 (br s, 1H), 8.42 (s, 1H), 8.38-8.32 (m, 1H), 8.26-8.15 (m, 2H), 8.08-7.98 (m, 2H), 7.79-7.69 (m, 1H), 7.41-7.18 (m, 3H), 5.17-4.97 (m, 1H), 4.47 (s, 2H), 3.99 (s, 3H), 3.87-3.69 (m, 1H), 3.20 (br d, J=11.6 Hz, 2H), 2.80-2.64 (m, 2H), 2.11 (br d, J=11.5 Hz, 1H), 1.94-1.66 (m, 3H), 1.24-1.06 (m, 1H).

### Synthesis of (R)-2-fluoro-4-(1-methyl-1H-1,2,3-triazol-4-yl)-N-(2-(4-(N-methylsulfamoyl)phenyl)thieno[3,2-c]pyridin-4-yl)-N-(piperidin-3-yl)benzamide (Example 534)

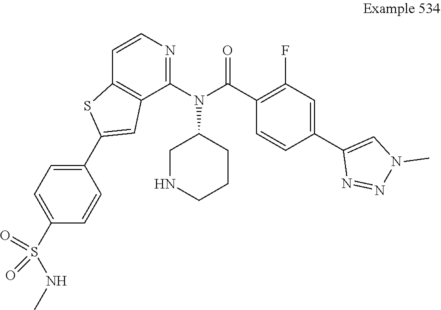 [SMILES: CNS(=O)(=O)c1ccc(-c2cc3c(N(C(=O)c4ccc(-c5cn(C)nn5)cc4F)[C@@H]4CCCNC4)nccc3s2)cc1]

Example 534 was prepared according to the procedure described for Example 363 by substituting 4-pyridylboronic acid with 2,2,2-trifluoro-1-[4-(4,4,5,5-tetramethyl-1,3,2-dioxaborolan-2-yl)phenyl]ethanol, provided the title compound hydrochloride salt (25%) as a white solid. LC-MS: [M+H⁺]=606.4. ¹H NMR (400 MHZ, CD₃OD): δ=8.39 (br d, J=5.4 Hz, 1H), 8.18 (br s, 1H), 8.01-7.88 (m, 6H), 7.4-7.15 (m, 3H), 5.26 (br s, 1H), 4.06 (s, 3H), 3.90 (br d, J=11.5 Hz, 1H), 3.78-3.56 (m, 1H), 3.35 (br d, J=12.4 Hz, 1H), 2.89 (br d, J=7.4 Hz, 1H), 2.59 (s, 3H), 2.09 (br d, J=9.9 Hz, 1H), 1.94 (br s, 2H), 1.50-1.22 (m, 1H).

### Synthesis of 2-fluoro-N-[2-[3-(methylsulfamoyl)phenyl]thieno[3,2-c]pyridin-4-yl]-4-(1-methyltriazol-4-yl)-N-[(3R)-3-piperidyl]benzamide (Example 535)

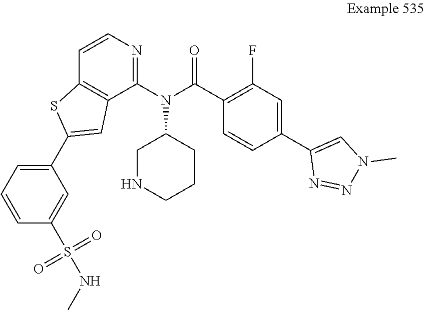 [SMILES: CNS(=O)(=O)c1cccc(-c2cc3c(N(C(=O)c4ccc(-c5cn(C)nn5)cc4F)[C@@H]4CCCNC4)nccc3s2)c1]

Example 535 was prepared according to the procedure described for Example 363 by substituting 4-pyridylboronic acid with N-methyl-3-(4,4,5,5-tetramethyl-1,3,2-dioxaborolan-2-yl)benzenesulfonamide, provided the title compound as a yellow solid. LC-MS: [M+H⁺]=606.3. ¹H NMR (400 MHZ, CD₃OD): δ 8.39 (d, J=5.5 Hz, 1H), 8.17 (s, 2H), 8.02 (br d, J=7.6 Hz, 1H), 7.94-7.87 (m, 2H), 7.85 (s, 1H), 7.78-7.69 (m, 1H), 7.38-7.27 (m, 2H), 7.19 (br d, J=7.3 Hz, 1H), 5.34-5.19 (m, 1H), 4.06 (s, 3H), 3.90 (br d, J=10.5 Hz, 1H), 3.77-3.63 (m, 2H), 3.37 (br s, 1H), 2.93-2.82 (m, 1H), 2.61 (s, 3H), 2.12-2.02 (m, 1H), 2.00-1.85 (m, 2H), 1.47-1.27 (m, 1H).

### Synthesis of N-[2-[4-(2-aminoethylsulfamoyl) phenyl]thieno[3,2-c]pyridin-4-yl]-2-fluoro-4-(1-methyltriazol-4-yl)-N-[(3R)-3-piperidyl] benzamide (Example 536)

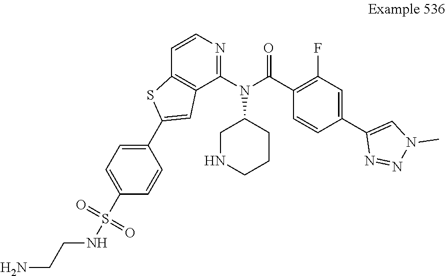 [SMILES: Cn1cc(-c2ccc(C(=O)N(c3nccc4sc(-c5ccc(S(=O)(=O)NCCN)cc5)cc34)[C@@H]3CCCNC3)c(F)c2)nn1]

Example 536 was prepared according to the procedure described for Example 363 by substituting 4-pyridylboronic acid with [4-[2-(tert-butoxycarbonylamino)ethylsulfamoyl]-phenyl]boronic acid, provided the title compound as a yellow solid. LC-MS: [M+H⁺]=635.4.

¹H NMR (400 MHZ, CD3OD): δ 8.40 (d, J=5.4 Hz, 1H), 8.19 (s, 1H), 8.01 (s, 4H), 7.97-7.88 (m, 2H), 7.37-7.25 (m, 2H), 7.23-7.14 (m, 1H), 5.33-5.20 (m, 1H), 4.06 (s, 3H), 3.90 (br d, J=11.1 Hz, 1H), 3.77-3.63 (m, 1H), 3.42-3.32 (m, 2H), 3.21-3.14 (m, 2H), 3.14-3.07 (m, 2H), 2.94-2.82 (m, 1H), 2.15-2.02 (m, 1H), 1.94 (br s, 2H), 1.47-1.27 (m, 1H).

### Synthesis of 2-fluoro-N-[2-[4-(isopropylsulfamoyl)phenyl]thieno[3,2-c]pyridin-4-yl]-4-(1-methyltriazol-4-yl)-N-[(3R)-3-piperidyl]benzamide (Example 537

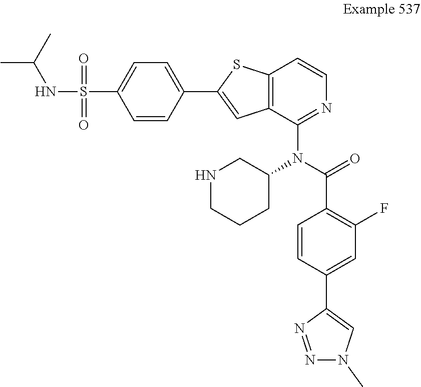 [SMILES: CC(C)NS(=O)(=O)c1ccc(-c2cc3c(N(C(=O)c4ccc(-c5cn(C)nn5)cc4F)[C@@H]4CCCNC4)nccc3s2)cc1]

Example 537 was prepared according to the procedure described for Example 363 by substituting 4-pyridylboronic acid with [4-(isopropylsulfamoyl)phenyl]boronic acid, provided the title compound hydrochloride salt as a white solid. LC-MS: [M+H⁺]=634.6. ¹H NMR (400 MHZ, CD3OD): δ 8.38 (d, J=5.5 Hz, 1H), 8.17 (s, 1H), 8.03-7.94 (m, 4H), 7.93-7.80 (m, 2H), 7.43-7.14 (m, 3H), 5.33-5.16 (m, 1H), 4.06 (s, 3H), 3.95-3.81 (m, 1H), 3.67 (br t, J=11.6 Hz, 1H), 3.43 (td, J=6.5, 13.1 Hz, 1H), 3.36 (br d, J=3.8 Hz, 1H), 2.96-2.79 (m, 1H), 2.51-1.80 (m, 3H), 1.49-1.27 (m, 1H), 1.11-0.99 (m, 6H).

### Synthesis of 2-fluoro-4-(1-methyltriazol-4-yl)-N-[2-(1-oxoisoindolin-5-yl)thieno[3,2-c]pyridin-4-yl]-N-[(3R)-3-piperidyl]benzamide (Example 538)

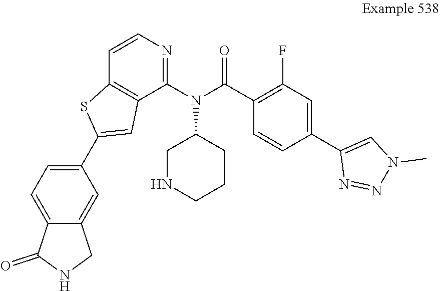 [SMILES: Cn1cc(-c2ccc(C(=O)N(c3nccc4sc(-c5ccc6c(c5)CNC6=O)cc34)[C@@H]3CCCNC3)c(F)c2)nn1]

Example 538 was prepared according to the procedure described for Example 363 by substituting 4-pyridylboronic acid with 5-(4,4,5,5-tetramethyl-1,3,2-dioxaborolan-2-yl)isoindolin-1-one, provided the title compound hydrochloride salt as a white solid. LC-MS: [M+H]⁺=568.3. ¹H NMR (400 MHZ, DMSO-d₆): δ 9.17-8.85 (m, 2H), 8.71 (s, 1H), 8.42 (s, 1H), 8.38-8.33 (m, 1H), 8.20-8.09 (m, 2H), 8.05-7.94 (m, 2H), 7.83-7.77 (m, 1H), 7.41-7.20 (m, 3H), 5.17-4.92 (m, 1H), 4.49 (br s, 2H), 3.99 (s, 3H), 3.71 (br d, J=11.1 Hz, 1H), 3.32-3.09 (m, 2H), 2.82-2.70 (m, 1H), 2.12-2.01 (m, 1H), 1.96-1.68 (m, 3H), 1.15 (dt, J=6.3, 11.6 Hz, 1H).

### Synthesis of 2-fluoro-N-[2-[3-(isopropylcarbamoyl)phenyl]thieno[3,2-c]pyridin-4-yl]-4-(1-methyltriazol-4-yl)-N-[(3R)-3-piperidyl]benzamide (Example 539)

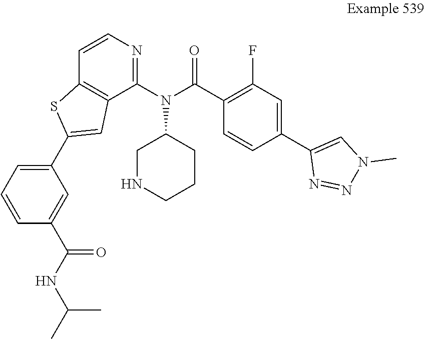 [SMILES: CC(C)NC(=O)c1cccc(-c2cc3c(N(C(=O)c4ccc(-c5cn(C)nn5)cc4F)[C@@H]4CCCNC4)nccc3s2)c1]

Example 539 was prepared according to the procedure described for Example 540 by substituting aniline with propan-2-amine, provided the title compound hydrochloride salt as a white solid. LC-MS: [M+H]⁺=598.4. ¹H NMR (400 MHZ, DMSO-d₆): δ 9.10-8.81 (m, 2H), 8.50-8.40 (m, 2H), 8.39-8.32 (m, 1H), 8.24-8.16 (m, 1H), 8.09 (s, 1H), 8.02 (br d, J=5.0 Hz, 2H), 7.94-7.88 (m, 1H), 7.66-7.58 (m, 1H), 7.41-7.18 (m, 3H), 5.18-4.93 (m, 1H), 4.17-4.11 (m, 1H), 3.99 (s, 3H), 3.73 (br d, J=10.8 Hz, 1H), 3.56-3.56 (m, 2H), 3.40-3.32 (m, 1H), 3.24-3.13 (m, 1H), 2.77-2.69 (m, 1H), 2.10-2.01 (m, 1H), 1.96-1.70 (m, 2H), 1.21 (d, J=6.6 Hz, 6H).

### Synthesis of 2-fluoro-4-(1-methyltriazol-4-yl)-N-[2-[3-(phenylcarbamoyl)phenyl]thieno[3,2-c]pyridin-4-yl]-N-[(3R)-3-piperidyl]benzamide (Example 540)

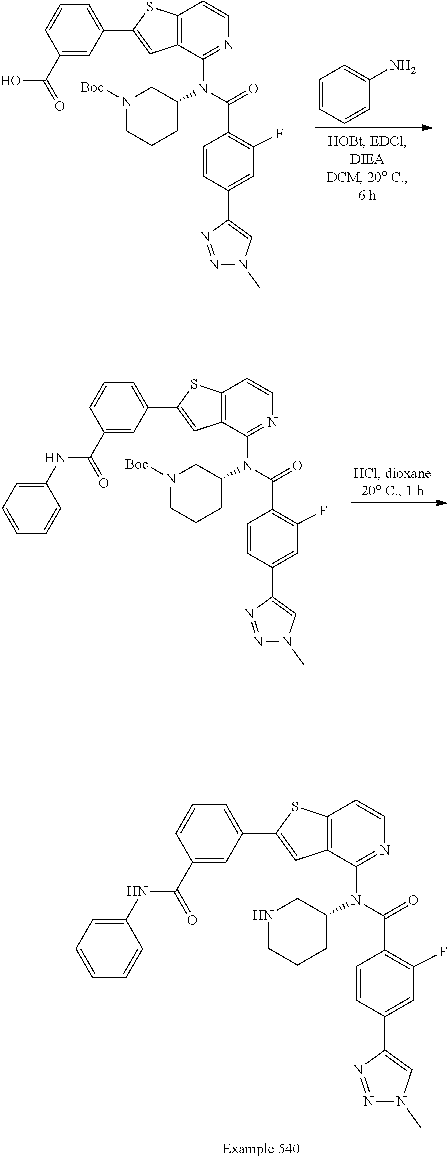 [SMILES: Cn1cc(-c2ccc(C(=O)N(c3nccc4sc(-c5cccc(C(=O)O)c5)cc34)[C@@H]3CCCN(C(=O)OC(C)(C)C)C3)c(F)c2)nn1 ; Nc1ccccc1 ; Cn1cc(-c2ccc(C(=O)N(c3nccc4sc(-c5cccc(C(=O)Nc6ccccc6)c5)cc34)[C@@H]3CCCN(C(=O)OC(C)(C)C)C3)c(F)c2)nn1 ; Cn1cc(-c2ccc(C(=O)N(c3nccc4sc(-c5cccc(C(=O)Nc6ccccc6)c5)cc34)[C@@H]3CCCNC3)c(F)c2)nn1]

### Step 1:

To a solution of 3-[4-[[(3R)-1-tert-butoxycarbonyl-3-piperidyl]-[2-fluoro-4-(1-methyltriazol-4-yl)benzoyl]amino]thieno[3,2-c]pyridin-2-yl]benzoic acid (87.15 mg, 119.44 μmol, 1 eq) in DCM (2 mL) was added HOBt (24.21 mg, 1.5 eq) and EDCI (34.34 mg, 1.5 eq), then DIEA (30.87 mg, 238.88 μmol, 2 eq) and aniline (16.68 mg, 179.16 μmol, 1.5 eq) was added. The resulting mixture was stirred at 20° C. for 2 h. The reaction mixture was concentrated under reduced pressure to give crude tert-butyl (3R)-3-[[2-fluoro-4-(1-methyltriazol-4-yl)benzoyl]-[2-[3-(phenylcarbamoyl)phenyl]thieno[3,2-c]pyridin-4-yl]amino]piperidine-1-carboxylate (110 mg, crude) as a yellow solid, which was used for next step directly without purification.

### Step 2:

The title compound hydrochloride salt was obtained as an off-white solid by deprotection Method A used in Example 97. LC-MS: [M+H]⁺=632.4. ¹H NMR (400 MHZ, DMSO-d₆): δ 10.53-10.43 (m, 1H), 9.07-8.79 (m, 2H), 8.45-8.31 (m, 3H), 8.17 (s, 1H), 8.13-8.00 (m, 3H), 7.82 (br d, J=7.9 Hz, 2H), 7.75-7.66 (m, 1H), 7.39 (br t, J=7.8 Hz, 3H), 7.33 (br d, J=8.1 Hz, 1H), 7.29-7.21 (m, 1H), 7.14 (t, J=7.4 Hz, 1H), 5.19-4.92 (m, 1H), 3.99 (s, 3H), 3.73 (br d, J=11.0 Hz, 1H), 3.38-3.38 (m, 2H), 3.24-3.14 (m, 1H), 2.71 (br d, J=13.0 Hz, 1H), 2.08 (br d, J=10.4 Hz, 1H), 1.98-1.63 (m, 3H), 1.24-1.09 (m, 1H).

### Synthesis of 2-fluoro-N-[2-[3-(methylcarbamoyl)phenyl] thieno[3, 2-c]pyridin-4-yl]-4-(1-methyltriazol-4-yl)-N-[(3R)-3-piperidyl] benzamide (Example 541)

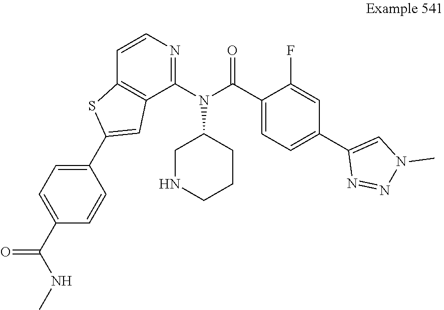 [SMILES: CNC(=O)c1ccc(-c2cc3c(N(C(=O)c4ccc(-c5cn(C)nn5)cc4F)[C@@H]4CCCNC4)nccc3s2)cc1]

Example 541 was prepared according to the procedure described for Example 540 by substituting aniline with methylamine, provided the title compound hydrochloride salt as a white solid. LC-MS: [M+H]=570.3. ¹H NMR (400 MHZ, DMSO-d₆): δ 9.16-8.77 (m, 2H), 8.74-8.56 (m, 1H), 8.47-8.39 (m, 1H), 8.38-8.31 (m, 1H), 8.29-8.21 (m, 1H), 8.10 (s, 1H), 8.07-7.97 (m, 2H), 7.94-7.86 (m, 1H), 7.69-7.58 (m, 1H), 7.42-7.17 (m, 3H), 5.21-4.89 (m, 1H), 4.07-3.94 (m, 3H), 3.83-3.67 (m, 1H), 3.25-3.14 (m, 1H), 2.85 (d, J=4.5 Hz, 3H), 2.79-2.69 (m, 1H), 2.29-2.00 (m, 1H), 1.96-1.67 (m, 3H), 1.30-0.99 (m, 1H). ¹⁹F NMR (376 MHZ, DMSO-d6): δ −113.60 (br s, 1F).

### Synthesis of 2-fluoro-N-[2-[3-(2-hydroxyethylcarbamoyl)phenyl]thieno[3,2-c]pyridin-4-yl]-4-(1-methyltriazol-4-yl)-N-[(3R)-3-piperidyl]benzamide (Example 542)

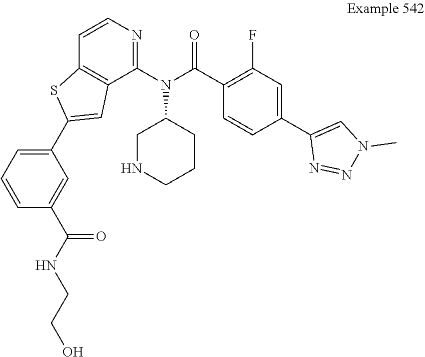 [SMILES: Cn1cc(-c2ccc(C(=O)N(c3nccc4sc(-c5cccc(C(=O)NCCO)c5)cc34)[C@@H]3CCCNC3)c(F)c2)nn1]

Example 541 was prepared according to the procedure described for Example 540 by substituting aniline with 2-aminoethanol, provided the title compound hydrochloride salt as a white solid. LC-MS: [M+H]=600.4. ¹H NMR (400 MHZ, DMSO-d₆): δ 8.67 (br t, J=5.1 Hz, 1H), 8.43-8.29 (m, 2H), 8.23 (s, 1H), 8.05-7.87 (m, 4H), 7.62 (br t, J=7.6 Hz, 1H), 7.36-7.16 (m, 2H), 4.81-4.60 (m, 2H), 4.19-3.91 (m, 3H), 3.56 (q, J=5.8 Hz, 2H), 3.39 (q, J=6.1 Hz, 2H), 3.25-3.17 (m, 1H), 3.00-2.65 (m, 2H), 2.17 (br d, J=11.5 Hz, 1H), 1.98-1.76 (m, 1H), 1.70-1.42 (m, 2H), 1.00 (br d, J=9.5 Hz, 1H).

### Synthesis of 2-fluoro-N-[2-(1H-indol-6-yl)thieno [3, 2-c] pyridin-4-yl]-4-(1-methyltriazol-4-yl)-N-[(3R)-3-piperidyl] benzamide (Example 543)

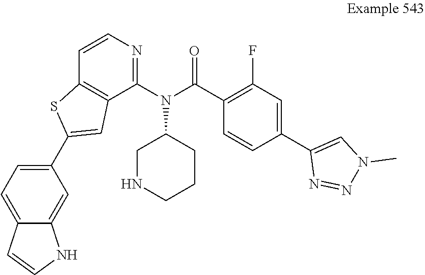 [SMILES: Cn1cc(-c2ccc(C(=O)N(c3nccc4sc(-c5ccc6cc[nH]c6c5)cc34)[C@@H]3CCCNC3)c(F)c2)nn1]

Example 543 was prepared according to the procedure described for Example 363 by substituting 4-pyridylboronic acid with 1H-indol-6-ylboronic acid, provided the title compound hydrochloride salt as a white solid. LC-MS: [M+H⁺]=552.4. ¹H NMR (400 MHZ, CD₃OD): δ 8.30 (d, J=5.5 Hz, 1H), 8.15 (s, 1H), 7.91-7.74 (m, 2H), 7.68-7.61 (m, 2H), 7.44 (br d, J=8.1 Hz, 1H), 7.41-7.32 (m, 2H), 7.29 (br d, J=8.0 Hz, 1H), 7.24-7.12 (m, 1H), 6.52 (d, J=2.9 Hz, 1H), 5.32-5.14 (m, 1H), 4.04 (s, 3H), 3.89 (br d, J=11.1 Hz, 1H), 3.66 (br t, J=11.8 Hz, 1H), 3.36 (br s, 1H), 2.87 (dt, J=4.2, 11.7 Hz, 1H), 2.15-1.82 (m, 3H), 1.47-1.27 (m, 1H).

### Synthesis of 2-fluoro-N-[2-(1H-indol-6-yl)thieno [3, 2-c] pyridin-4-yl]-4-(1-methyltriazol-4-yl)-N-[(3R)-3-piperidyl] benzamide (Example 544)

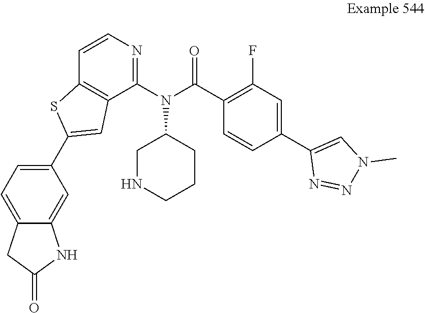 [SMILES: Cn1cc(-c2ccc(C(=O)N(c3nccc4sc(-c5ccc6c(c5)NC(=O)C6)cc34)[C@@H]3CCCNC3)c(F)c2)nn1]

Example 544 was prepared according to the procedure described for Example 363 by substituting 4-pyridylboronic acid with 6-(4,4,5,5-tetramethyl-1,3,2-dioxaborolan-2-yl)indolin-2-one, provided the title compound hydrochloride salt as a white solid. LC-MS: [M+H⁺]=568.3. ¹H NMR (400 MHZ, CD₃OD): δ 8.37 (d, J=5.4 Hz, 1H), 8.18 (s, 1H), 7.87 (br d, J=5.1 Hz, 1H), 7.69 (s, 1H), 7.46-7.25 (m, 5H), 7.23-7.13 (m, 1H), 5.34-5.21 (m, 1H), 4.08 (s, 3H), 3.96-3.82 (m, 1H), 3.74-3.58 (m, 1H), 3.23-3.09 (m, 1H), 2.95-2.83 (m, 1H), 2.11-2.01 (m, 1H), 1.99-1.81 (m, 2H), 1.31 (s, 3H).

### Synthesis of tert-butyl (3R)-3-[[2-fluoro-4-(1-methyltriazol-4-yl) benzoyl]-[2-(2-oxo-1, 3-dihydrobenzimidazol-5-yl)thieno [3, 2-c] pyridin-4-yl] amino] piperidine-1-carboxylate (Example 545)

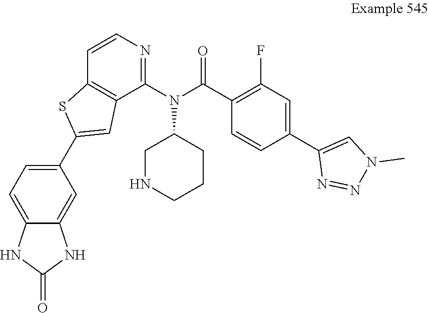 [SMILES: Cn1cc(-c2ccc(C(=O)N(c3nccc4sc(-c5ccc6[nH]c(=O)[nH]c6c5)cc34)[C@@H]3CCCNC3)c(F)c2)nn1]

Example 545 was prepared according to the procedure described for Example 363 by substituting 4-pyridylboronic acid with 5-(4,4,5,5-tetramethyl-1,3,2-dioxaborolan-2-yl)-1,3-dihydrobenzimidazol-2-one, provided the title compound hydrochloride salt (37%) as a white solid. LC-MS: [M+H⁺]=569.3. ¹H NMR (400 MHZ, CD₃OD): δ 8.32 (d, J=5.5 Hz, 1H), 8.17 (s, 1H), 7.84 (br d, J=5.3 Hz, 1H), 7.61 (s, 1H), 7.51-7.40 (m, 2H), 7.39-7.25 (m, 2H), 7.23-7.09 (m, 2H), 5.35-5.16 (m, 1H), 4.05 (s, 3H), 3.89 (br d, J=11.9 Hz, 1H), 3.66 (br t, J=11.7 Hz, 1H), 3.39-3.33 (m, 1H), 2.94-2.81 (m, 1H), 2.16-1.83 (m, 3H), 1.57-1.27 (m, 1H).

### Synthesis of 2-fluoro-N-[2-(1H-indol-5-yl)thieno[3,2-c]pyridin-4-yl]-4-(1-methyltriazol-4-yl)-N-[(3R)-3-piperidyl]benzamide (Example 546)

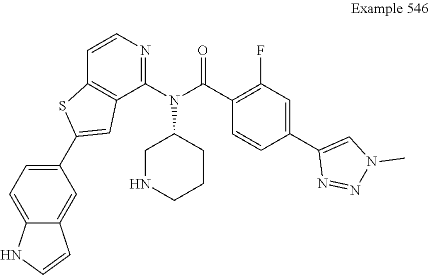 [SMILES: Cn1cc(-c2ccc(C(=O)N(c3nccc4sc(-c5ccc6[nH]ccc6c5)cc34)[C@@H]3CCCNC3)c(F)c2)nn1]

Example 546 was prepared according to the procedure described for Example 363 by substituting 4-pyridylboronic acid with 1H-indol-5-ylboronic acid, provided the title compound hydrochloride salt as a yellow solid. LC-MS: [M+H⁺]=552.2. ¹H NMR (400 MHz, CD₃OD): δ 8.29-8.20 (m, 1H), 8.14 (s, 1H), 7.95 (br s, 1H), 7.77 (br d, J=5.1 Hz, 1H), 7.61-7.47 (m, 3H), 7.37-7.23 (m, 2H), 7.23-7.14 (m, 1H), 6.56 (d, J=3.1 Hz, 1H), 4.04 (s, 3H), 3.65-3.55 (m, 1H), 3.46-3.37 (m, 1H), 3.27-3.18 (m, 1H), 3.05-2.92 (m, 1H), 2.53-2.32 (m, 1H), 2.10-1.91 (m, 1H), 1.87-1.63 (m, 2H), 1.41-1.25 (m, 1H).

### Synthesis of (R)—N-(2-(4-acetamidophenyl)thieno[3,2-c]pyridin-4-yl)-2-fluoro-4-(1-methyl-1H-1,2,3-triazol-4-yl)-N-(piperidin-3-yl)benzamide (Example 547)

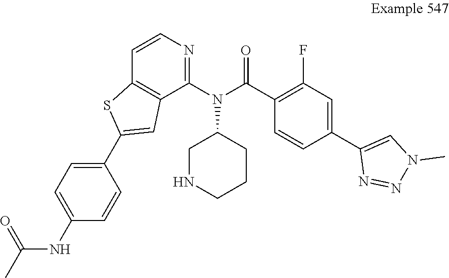 [SMILES: CC(=O)Nc1ccc(-c2cc3c(N(C(=O)c4ccc(-c5cn(C)nn5)cc4F)[C@@H]4CCCNC4)nccc3s2)cc1]

Example 547 was prepared according to the procedure described for Example 363 by substituting 4-pyridylboronic acid with N-[4-(4,4,5,5-tetramethyl-1,3,2-dioxaborolan-2-yl)phenyl]acetamide, provided the title compound hydrochloride salt as a white solid. LC-MS: [M+H⁺]=570.3. ¹H NMR (400 MHZ, CD₃OD): δ 8.32 (d, J=5.5 Hz, 1H), 8.17 (s, 1H), 7.84 (br d, J=5.4 Hz, 1H), 7.71 (s, 4H), 7.68-7.59 (m, 1H), 7.38-7.26 (m, 2H), 7.24-7.14 (m, 1H), 5.39-5.09 (m, 1H), 4.06 (s, 3H), 3.89 (br d, J=9.8 Hz, 1H), 3.65 (br t, J=11.6 Hz, 1H), 3.36 (br s, 1H), 2.97-2.80 (m, 1H), 2.17 (s, 3H), 2.11-1.84 (m, 3H), 1.50-1.24 (m, 1H).

### Synthesis of (R)-2-fluoro-4-(1-methyl-1H-1,2,3-triazol-4-yl)-N-(2-(4-(3-methylthioureido)phenyl)thieno[3,2-c]pyridin-4-yl)-N-(piperidin-3-yl)benzamide (Example 548)

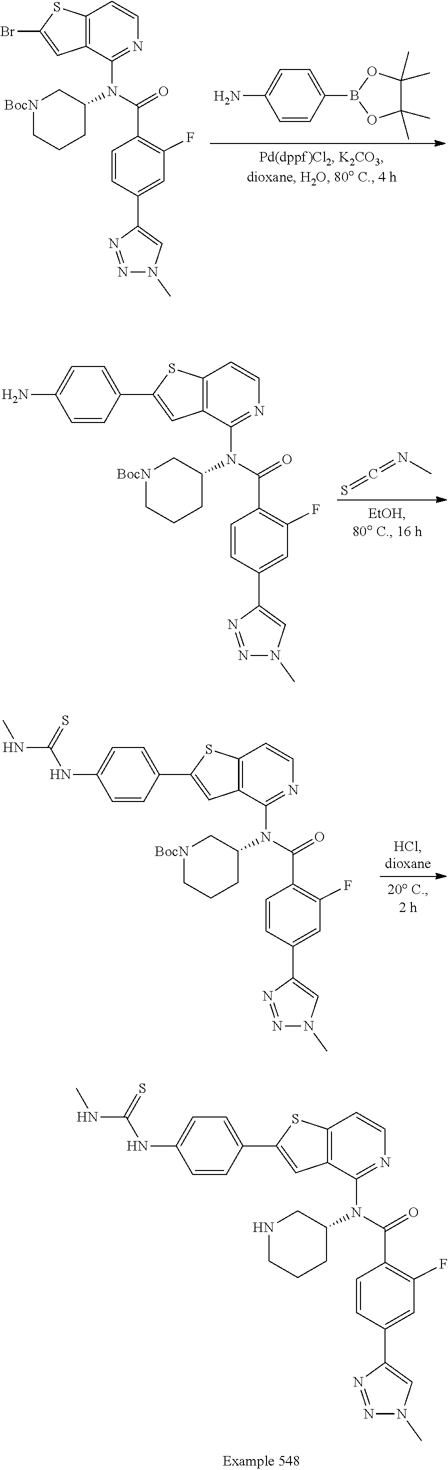

### Step 1:

To a solution of tert-butyl (3R)-3-[(2-bromothieno [3, 2-c] pyridin-4-yl)-[2-fluoro-4-(1-methyltriazol-4-yl) benzoyl] amino] piperidine-1-carboxylate (200 mg, 325 μmol) and 4-(4,4,5,5-tetramethyl-1,3,2-dioxaborolan-2-yl) aniline (106.8 mg, 487.4 μmol) in dioxane (2 mL) and Water (0.5 mL) was added K₂CO₃ (134 mg, 975 μmol) and Pd(dppf)Cl₂ (23.8 mg, 32.5 μmol) at 20° C. under N₂. The mixture was heated to 80° C. and stirred for 4 h under N₂. The reaction mixture was filtered and concentrated under reduced pressure to give the residue. The residue was purified by prep-TLC (SiO₂, Ethylacetate:Petroleumether=1:1) to give tert-butyl (3R)-3-[[2-(4-aminophenyl)thieno [3, 2-c] pyridin-4-yl]-[2-fluoro-4-(1-methyltriazol-4-yl)benzoyl] amino] piperidine-1-carboxylate (173 mg, 84%) as a white solid.

### Step 2:

To a solution of tert-butyl (3R)-3-[[2-(4-aminophenyl) thieno [3, 2-c] pyridin-4-yl]-[2-fluoro-4-(1-methyltriazol-4-yl)benzoyl] amino] piperidine-1-carboxylate (173 mg, 275.60 μmo) and methylimino (thioxo) methane (24.18 mg, 330.72 μmol, 22.62 μL) in EtOH (3 mL) was heated to 80° C. and stirred for 16 h. The reaction mixture was filtered and concentrated under reduced pressure to give the crude. The crude was purified by prep-TLC (SiO₂, Petroleum ether:Ethyl acetate=0:1) to give tert-butyl (3R)-3-[[2-fluoro-4-(1-methyltriazol-4-yl) benzoyl]-[2-[4-(methylcarbamothioylamino)phenyl]thieno[3, 2-c]pyridin-4-yl]amino]piperidine-1-carboxylate (90 mg, 46%) as a white solid.

### Step 3:

The title compound hydrochloride salt was obtained as a white solid by deprotection Method A used in Example 97. LC-MS: [M+H⁺]=601.3. ¹H NMR (400 MHZ, CD₃OD): δ 8.33 (d, J=5.5 Hz, 1H), 8.17 (s, 1H), 7.85 (br d, J=5.3 Hz, 1H), 7.74 (br d, J=8.3 Hz, 2H), 7.70-7.64 (m, 1H), 7.55 (br d, J=8.1 Hz, 2H), 7.39-7.12 (m, 3H), 5.24 (br t, J=10.9 Hz, 1H), 4.06 (s, 3H), 3.89 (br d, J=11.5 Hz, 1H), 3.66 (br t, J=11.4 Hz, 1H), 3.36 (br s, 1H), 3.07 (s, 3H), 2.95-2.81 (m, 1H), 2.07 (br d, J=11.0 Hz, 1H), 1.99-1.80 (m, 2H), 1.50-1.25 (m, 1H).

### Synthesis of 2-fluoro-N-[2-[3-(methylamino)-3-oxo-prop-1-ynyl]thieno[3,2-c]pyridin-4-yl]-N-[(3R)-3-piperidyl]-4-(triazolo [4,5-b]pyridin-3-yl)benzamide (Example 549)

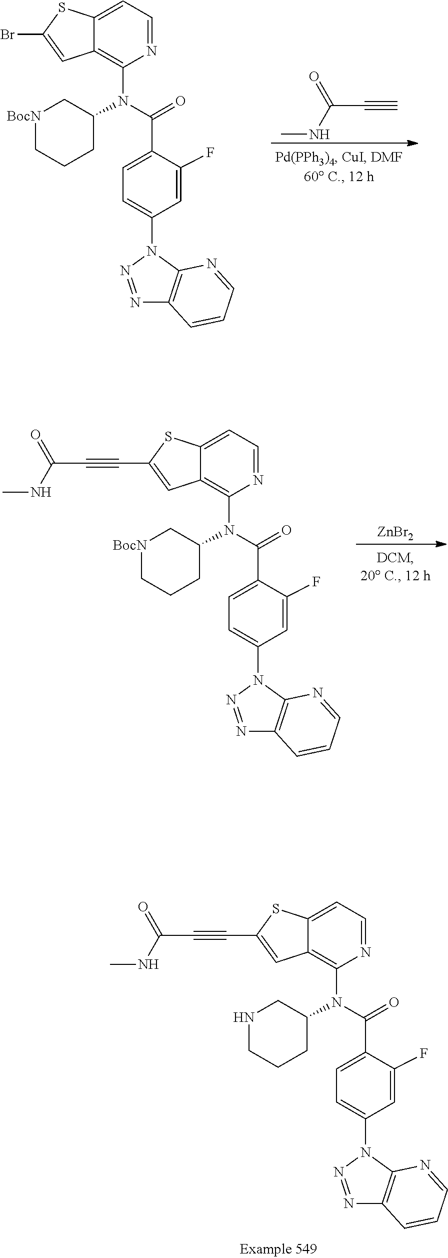

### Step 1:

A mixture of tert-butyl (3R)-3-[(2-bromothieno[3,2-c]pyridin-4-yl)-[2-fluoro-4-(triazolo[4,5-b]pyridin-3-yl)benzoyl]amino]piperidine-1-carboxylate (150 mg, 229.87 μmol), N-methylprop-2-ynamide (95.50 mg, 1.15 mmol), Pd(PPh₃)₄ (26.56 mg, 22.99 μmol), CuI (4.38 mg, 22.99 μmol) in DMF (2 mL) was purged with N₂ at 20° C., and then the mixture was heated to 60° C. and stirred for 12 h under N₂ atmosphere. The reaction mixture was poured into ice water 5 mL and filtered to give filter cake. The filter cake was purified by prep-TLC (SiO₂, Petroleum ether/Ethyl acetate=1:4) to give tert-butyl (3R)-3-[[2-fluoro-4-(triazolo[4,5-b]pyridin-3-yl)benzoyl]-[2-[3-(methylamino)-3-oxo-prop-1-ynyl]thieno[3,2-c]pyridin-4-yl]amino]piperidine-1-carboxylate (60 mg, 39%) as a yellow solid. LC-MS: [M+H⁺]=655.4.

### Step 2:

To a solution of tert-butyl (3R)-3-[[2-fluoro-4-(triazolo[4,5-b]pyridin-3-yl)benzoyl]-[2-[3-(methylamino)-3-oxo-prop-1-ynyl]thieno[3,2-c]pyridin-4-yl]amino]piperidine-1-carboxylate (57 mg, 87.06 μmol) in DCM (2 mL) was added ZnBr₂ (98.03 mg, 435.31 μmol). The mixture was stirred at 20° C. for 12 h. The reaction mixture was concentrated under reduced pressure. The residue was purified by Prep-HPLC (column: Waters Xbridge Prep OBD C18 150*40 mm*10 μm; mobile phase: [H₂O (10 mM NH₄HCO₃)-MeCN]; gradient: 15%-55% B over 8.0 min) to give the title compound (18 mg, 37%) as a white solid. LC-MS: [M+H⁺]=555.3.

1H NMR (400 MHZ, CD₃OD): δ 8.78 (br s, 1H), 8.63-8.36 (m, 2H), 8.19-7.75 (m, 3H), 7.66-7.29 (m, 2H), 4.61 (br s, 1H), 3.64-3.43 (m, 1H), 3.21-3.03 (m, 1H), 3.01-2.69 (m, 4H), 2.60-2.23 (m, 1H), 2.13-1.51 (m, 3H), 1.40-1.10 (m, 1H).

### Synthesis of 2-fluoro-N-[2-[3-(methylamino)-3-oxo-prop-1-ynyl]thieno[3,2-c]pyridin-4-yl]-4-(1-methyltriazol-4-yl)-N-[(3R)-3-piperidyl] benzamide (Example 550)

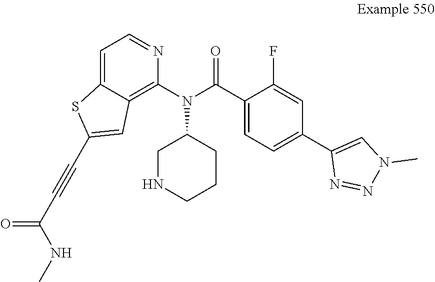 [SMILES: CNC(=O)C#Cc1cc2c(N(C(=O)c3ccc(-c4cn(C)nn4)cc3F)[C@@H]3CCCNC3)nccc2s1]

Example 550 was prepared according to the procedure described for Example 549 by substituting tert-butyl (3R)-3-[(2-bromothieno[3,2-c]pyridin-4-yl)-[2-fluoro-4-(triazolo[4,5-b]pyridin-3-yl)benzoyl]amino]piperidine-1-carboxylate with (3R)-3-[(2-bromothieno[3,2-c]pyridin-4-yl)-[2-fluoro-4-(1-methyltriazol-4-yl)benzoyl]amino]piperidine-1-carboxylate, provided the title compound as a yellow solid. LC-MS: [M+H⁺]=518.2. ¹H NMR (400 MHZ, CD₃OD): δ 8.54-8.32 (m, 1H), 8.30-8.09 (m, 1H), 7.94-7.65 (m, 2H), 7.57-7.05 (m, 3H), 4.59 (br s, 1H), 4.30-3.90 (m, 3H), 3.63-3.46 (m, 1H), 3.23-3.06 (m, 1H), 3.02-2.91 (m, 1H), 2.89-2.74 (m, 3H), 2.50-2.25 (m, 1H), 2.12-1.90 (m, 1H), 1.85-1.52 (m, 2H), 1.46-1.10 (m, 1H).

### Synthesis of 2-fluoro-N-[2-(3-hydroxy-3-methyl-but-1-ynyl)thieno[3,2-c]pyridin-4-yl]-4-(1-methyltriazol-4-yl)-N-[(3R)-3-piperidyl]benzamide (Example 551)

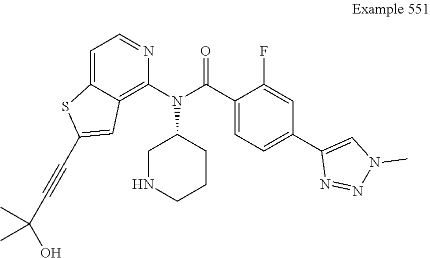 [SMILES: Cn1cc(-c2ccc(C(=O)N(c3nccc4sc(C#CC(C)(C)O)cc34)[C@@H]3CCCNC3)c(F)c2)nn1]

Example 551 was prepared according to the procedure described for Example 550 by substituting N-methylprop-2-ynamide with 2-methylbut-3-yn-2-ol, provided the title compound as a white solid. LC-MS: [M+H+]=519.4. ¹H NMR (400 MHZ, CD₃OD): δ 8.40-8.13 (m, 2H), 7.76 (br s, 1H), 7.52 (s, 1H), 7.41-7.23 (m, 2H), 7.15 (br d, J=2.4 Hz, 1H), 4.77-4.54 (m, 1H), 4.09 (br s, 3H), 3.59-3.40 (m, 1H), 3.17-2.85 (m, 2H), 2.57-2.22 (m, 2H), 2.04-1.77 (m, 1H), 1.76-1.51 (m, 8H), 1.34-1.09 (m, 1H).

### Synthesis of 4-[[2-fluoro-4-(1-methyltriazol-4-yl) benzoyl]-[(3R)-3-piperidyl] amino] thieno [3, 2-c] pyridine-2-carboxamide (Example 552)

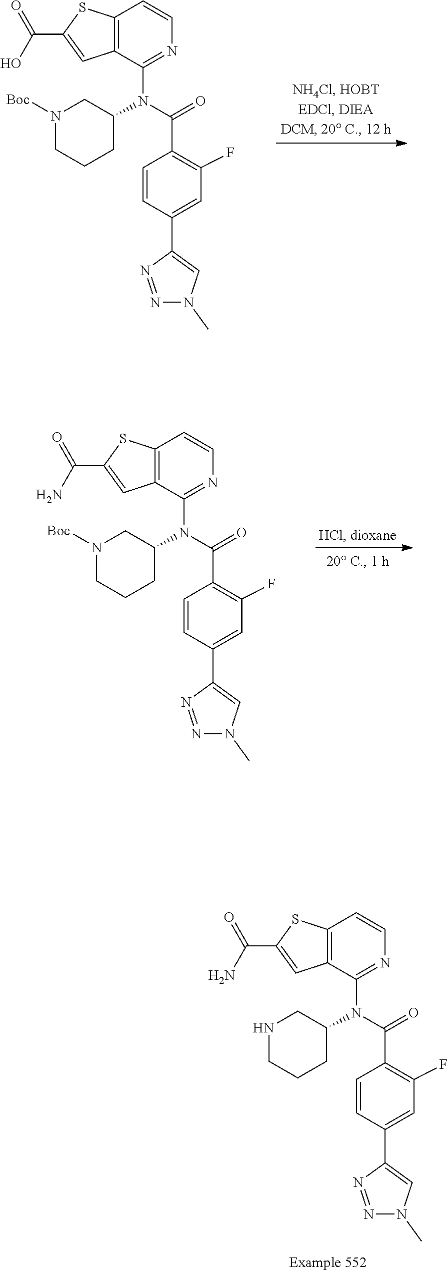 [SMILES: Cn1cc(-c2ccc(C(=O)N(c3nccc4sc(C(=O)O)cc34)[C@@H]3CCCN(C(=O)OC(C)(C)C)C3)c(F)c2)nn1 ; Cn1cc(-c2ccc(C(=O)N(c3nccc4sc(C(N)=O)cc34)[C@@H]3CCCN(C(=O)OC(C)(C)C)C3)c(F)c2)nn1 ; "HCl, dioxane" ; Cn1cc(-c2ccc(C(=O)N(c3nccc4sc(C(N)=O)cc34)[C@@H]3CCCNC3)c(F)c2)nn1]

### Step 1:

To a solution of 4-[[(3R)-1-tert-butoxycarbonyl-3-piperidyl]-[2-fluoro-4-(1-methyltriazol-4-yl)benzoyl]amino]thieno[3,2-c]pyridine-2-carboxylic acid (0.06 g, 103.34 μmol) in DCM (1 mL) was added HOBt (27.93 mg, 206.67 μmol) and EDCI (39.62 mg, 206.67 μmol) at 20° C., then the mixture was stirred at 20° C. for 5 min, then DIEA (26.71 mg, 206.67 μmol) and NH₄Cl (16.58 mg, 310.01 μmol) was added at 20° C., the mixture was stirred at 20° C. for 2 h. The mixture was concentrated under reduced pressure to give tert-butyl (3R)-3-[(2-carbamoylthieno [3, 2-c]pyridin-4-yl)-[2-fluoro-4-(1-methyltriazol-4-yl) benzoyl] amino] piperidine-1-carboxylate (500 mg, 83%) as a yellow solid.

### Step 2:

The title compound hydrochloride salt was obtained as an off-white solid by deprotection Method A used in Example 97. LC-MS: [M+H]=480.3. ¹H NMR (400 MHZ, DMSO-d₆): δ 9.11-8.70 (m, 2H), 8.43-8.27 (m, 2H), 8.26-8.14 (m, 1H), 8.10-7.87 (m, 2H), 7.74-7.38 (m, 2H), 7.34-7.11 (m, 2H), 5.23-4.69 (m, 1H), 4.01 (br s, 4H), 3.39-3.07 (m, 2H), 2.89-2.60 (m, 2H), 2.03-1.85 (m, 1H), 1.83-1.68 (m, 1H), 1.31-0.97 (m, 1H). ¹⁹F NMR (376 MHz, DMSO-d6): δ −113.38 (br s, 1F).

### Synthesis of N-[2-(6-amino-3-pyridyl)thieno[3,2-c]pyridin-4-yl]-2-fluoro-N-[(3R)-3-piperidyl]-4-(triazolo[4,5-b] pyridin-3-yl)benzamide (Example 553)

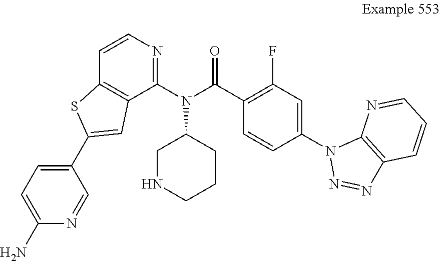 [SMILES: Nc1ccc(-c2cc3c(N(C(=O)c4ccc(-n5nnc6cccnc65)cc4F)[C@@H]4CCCNC4)nccc3s2)cn1]

Example 553 was prepared according to the procedure described for Example 161 by substituting 4-pyridylboronic acid with 5-(4,4,5,5-tetramethyl-1,3,2-dioxaborolan-2-yl)pyridin-2-amine, provided the title compound hydrochloride salt as a white solid. LC-MS: [M+H+]=566.3. ¹H NMR (400 MHZ, CD₃OD): δ 8.75 (br d, J=3.8 Hz, 1H), 8.51 (br d, J=8.1 Hz, 1H), 8.45-8.36 (m, 2H), 8.34-8.24 (m, 1H), 8.12-7.96 (m, 2H), 7.95-7.85 (m, 2H), 7.58-7.40 (m, 2H), 7.20 (d, J=9.4 Hz, 1H), 5.27 (br s, 1H), 4.00-3.84 (m, 1H), 3.70 (br t, J=11.5 Hz, 1H), 3.43-3.33 (m, 1H), 3.00-2.81 (m, 1H), 1.95 (br s, 3H), 1.53-1.26 (m, 1H).

### Synthesis of (R)-4-(3H-[1,2,3]triazolo[4,5-b]pyridin-3-yl)-2-fluoro-N-(piperidin-3-yl)-N-(6-(pyridin-2-yl) isoquinolin-1-yl) benzamide (Example 556)

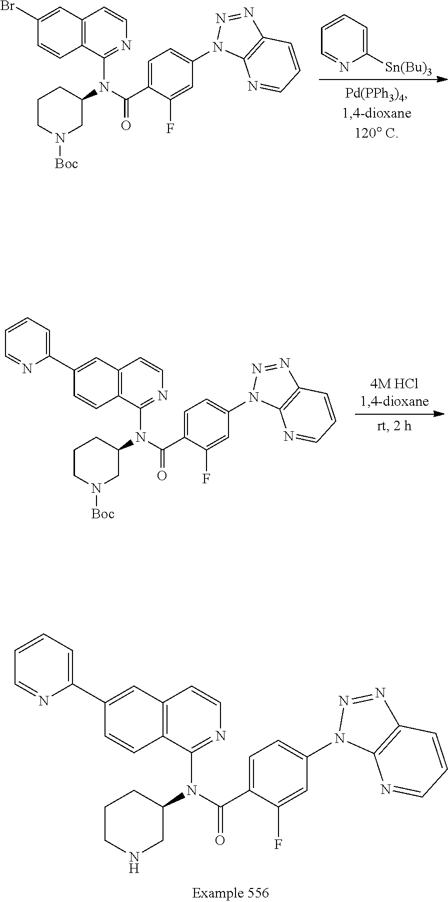

### Step 1:

A solution of tert-butyl (R)-3-(4-(3H-[1,2,3]triazolo[4,5-b]pyridin-3-yl)-N-(6-bromoisoquinolin-1-yl)-2-fluorobenzamido) piperidine-1-carboxylate (50 mg, 0.077 mmol) and 2-(tributylstannyl)pyridine (28.5 mg, 0.077 mmol) in 1,4-dioxane (1 mL) was degassed with argon for 10 min and followed by addition of tetra kiss (triphenylphosphine)palladium(0) (1.78 mg, 1.547 mmol). The reaction was heated and stirred at 90° C. for 16 h. The reaction mixture was diluted with water (30 mL) and extracted with EtOAc (2×50 mL). The combined organic layer was washed with brine (20 mL), dried over anhydrous Na₂SO₄, and concentrated under reduced pressure. The residue was purified by Prep-HPLC (Conditions: Kinetex evo C-18 (250×21.2×5 μm), 0.1% formic acid water, Mobile phase B: 100% MeCN, Time (min)/% B: 0/60, 10/80, MeCN+THF). The pure fractions were combined and concentrated under reduced pressure to afford tert-butyl (R)-3-(4-(3H-[1,2,3]triazolo[4,5-b]pyridin-3-yl)-2-fluoro-N-(6-(pyridin-2-yl)isoquinolin-1-yl)benzamido) piperidine-1-carboxylate (11 mg, 35.9%) as an off-white solid. LC-MS: [M+H⁺]=645.3.

### Step 2:

To a solution of tert-butyl (R)-3-(4-(3H-[1,2,3]triazolo[4,5-b]pyridin-3-yl)-2-fluoro-N-(6-(pyridin-2-yl) isoquinolin-1-yl)benzamido)piperidine-1-carboxylate (10 mg, 0.016 mmol) in DCM (1 mL) was added 4M HCl in 1,4-dioxane (0.5 mL, 16.46 mmol) at 0° C. and was stirred at room temperature for 2 h. The reaction mixture was concentrated under reduced pressure and dried by lyophilization to afford (R)-4-(3H-[1,2,3]triazolo[4,5-b]pyridin-3-yl)-2-fluoro-N-(piperidin-3-yl)-N-(6-(pyridin-2-yl)isoquinolin-1-yl) benzamide hydrochloride salt (8 mg, 84%) as an off-white solid. LC-MS: [M+H⁺]=545.2. ¹H NMR (CD₃OD, 400 MHZ): δ 8.89-8.88 (m, 1H), 8.73-8.66 (m, 3H), 8.50-8.48 (m, 4H), 8.39-8.31 (m, 1H), 8.01-7.98 (m, 4H), 7.54-7.51 (m, 1H), 7.38-7.34 (m, 1H), 5.38-5.36 (m, 1H), 4.10-4.00 (m, 1H), 3.81-3.76 (m, 1H), 2.98-2.96 (m, 1H), 2.08-1.94 (m, 3H), 1.37-1.28 (m, 1H), 0.94-0.91 (m, 1H).

### Synthesis of 2-fluoro-N-[2-(1H-indazol-5-yl)thieno[3,2-c]pyridin-4-yl]-N-[(3R)-3-piperidyl]-4-(triazolo[4,5-b]pyridin-3-yl)benzamide (Example 557)

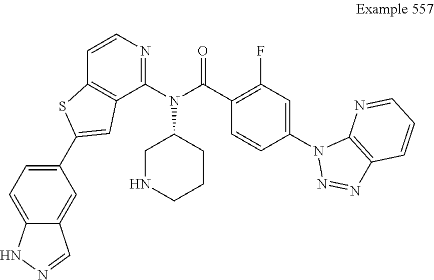 [SMILES: O=C(c1ccc(-n2nnc3cccnc32)cc1F)N(c1nccc2sc(-c3ccc4[nH]ncc4c3)cc12)[C@@H]1CCCNC1]

Example 557 was prepared according to the procedure described for Example 161 by substituting 4-pyridylboronic acid with 1H-indazol-5-ylboronic acid, provided the title compound hydrochloride salt (54%) as a light yellow solid. LC-MS: [M+H⁺]=590.20. ¹H NMR (400 MHZ, DMSO-d₆): δ 9.33 (br d, J=9.9 Hz, 1H), 9.17-9.00 (m, 1H), 8.77 (br d, J=4.1 Hz, 1H), 8.65 (d, J=8.3 Hz, 1H), 8.37-8.25 (m, 2H), 8.23-8.18 (m, 1H), 8.09-7.98 (m, 3H), 7.95-7.85 (m, 2H), 7.74-7.66 (m, 1H), 7.61-7.50 (m, 2H), 5.23-4.92 (m, 1H), 3.75 (br s, 1H), 3.40-3.27 (m, 1H), 3.19 (br d, J=10.4 Hz, 1H), 2.85-2.62 (m, 1H), 2.36-1.71 (m, 3H), 1.27-1.09 (m, 1H).

### Synthesis of (R)-2-fluoro-N-(8-methylisoquinolin-1-yl)-N-(piperidin-3-yl)-4-((4-(pyridin-4-yl)pyrimidin-2-yl)amino)benzamide (Example 558)

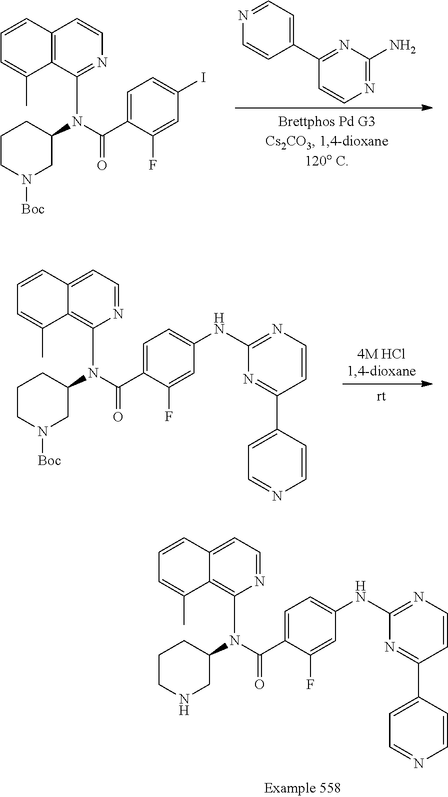 [SMILES: Cc1cccc2ccnc(N(C(=O)c3ccc(I)cc3F)[C@@H]3CCCN(C(=O)OC(C)(C)C)C3)c12 ; Nc1nccc(-c2ccncc2)n1 ; Cc1cccc2ccnc(N(C(=O)c3ccc(Nc4nccc(-c5ccncc5)n4)cc3F)[C@@H]3CCCN(C(=O)OC(C)(C)C)C3)c12 ; Cc1cccc2ccnc(N(C(=O)c3ccc(Nc4nccc(-c5ccncc5)n4)cc3F)[C@@H]3CCCNC3)c12]

### Step 1:

To a solution of tert-butyl (R)-3-(2-fluoro-4-iodo-N-(8-methylisoquinolin-1-yl)benzamido)piperidine-1-carboxylate (120 mg, 0.204 mmol) and 4-(pyridin-4-yl)pyrimidin-2-amine (35.1 mg, 0.204 mmol) in 1,4-dioxane (3 mL) was added Cs₂CO₃ (133 mg, 0.407 mmol) at room temperature. The reaction mixture was degassed with nitrogen for 5 min. Then, Brettphos Pd G3 (18.45 mg, 0.020 mmol) was added to reaction mixture and stirred at 100° C. for 16 h. The reaction mixture was cooled to room temperature, diluted with water, and extracted with EtOAc (2×20 mL). The combined organic layer was washed with brine, dried over Na₂SO₄, and concentrated. The residue was purified by Prep-HPLC (Condition: Luna OMEGA C-18 (250×21.2×5 μm), Mobile phase A: 0.1% formic acid in water (100%), Mobile phase B: 100% MeCN, Gradient: T/% B: 0/55, 10/80 Flow: 15 mL/min, Dilution: MeCN+H₂O+THF). Pure fractions were concentrated under reduced pressure to afford tert-butyl (R)-3-(2-fluoro-N-(8-methylisoquinolin-1-yl)-4-((4-(pyridin-4-yl)pyrimidin-2-yl)amino)benzamido)piperidine-1-carboxylate (30 mg, 24.75%) as an off-white solid. LC-MS: [M+H⁺]=634.35.

### Step 2:

The title compound hydrochloride salt was obtained as an off-white solid by deprotection Method A used in Example 97. LC-MS: [M+H⁺]=534.30. HPLC: 26.27%+72.19%. 1H NMR (CD₃OD, 400 MHZ): δ 9.03 (d, J=6 Hz, 2H), 8.73-8.71 (m, 3H), 8.47-8.42 (m, 1H), 7.79-7.63 (m, 3H), 7.55-7.46 (m, 3H), 6.94 (d, J=8.4 Hz, 1H), 6.76-6.72 (m, 1H), 5.10-5.04 (m, 1H), 3.96-3.95 (m, 1H), 3.86-3.81 (m, 1H), 3.44-3.39 (m, 1H), 3.03-3.00 (m, 1H), 2.82 (s, 3H), 2.71-2.68 (m, 1H), 1.95-1.78 (m, 2H), 1.60-1.56 (m, 1H).

### Synthesis of (R)-2-fluoro-N-(8-methylisoquinolin-1-yl)-4-((4-phenylpyrimidin-2-yl)amino)-N-(piperidin-3-yl)benzamide (Example 559)

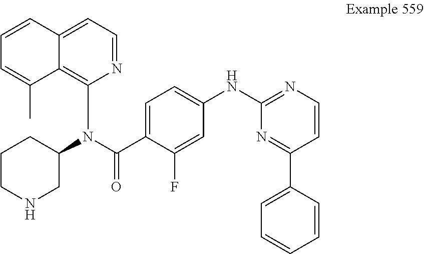 [SMILES: Cc1cccc2ccnc(N(C(=O)c3ccc(Nc4nccc(-c5ccccc5)n4)cc3F)[C@@H]3CCCNC3)c12]

Example 559 was prepared according to the procedure described for Example 558 by substituting 4-(pyridin-4-yl)pyrimidin-2-amine with 4-phenylpyrimidin-2-amine, provided the title compound hydrochloride salt as an off-white solid. LC-MS: [M+H⁺]=533.3. HPLC: 98.03%.

¹H NMR (CD₃OD, 400 MHZ): δ 8.46-8.42 (m, 2H), 8.10-8.08 (m, 2H), 7.75-7.44 (m, 9H), 6.92 (dd, J=2.0 Hz, J=8.8 Hz, 1H), 6.78-6.74 (m, 1H), 5.10-5.05 (m, 1H), 3.99-3.96 (m, 1H), 3.85-3.80 (m, 1H), 3.42-3.34 (m, 1H), 3.03-3.00 (m, 1H), 2.82 (s, 3H), 2.72-2.68 (m, 1H), 1.98-1.94 (m, 2H), 1.61-1.58 (m, 1H).

### Synthesis of (R)-2-fluoro-N-(6-fluoroisoquinolin-1-yl)-4-((4-morpholinopyrimidin-2-yl)amino)-N-(piperidin-3-yl)benzamide (Example 560)

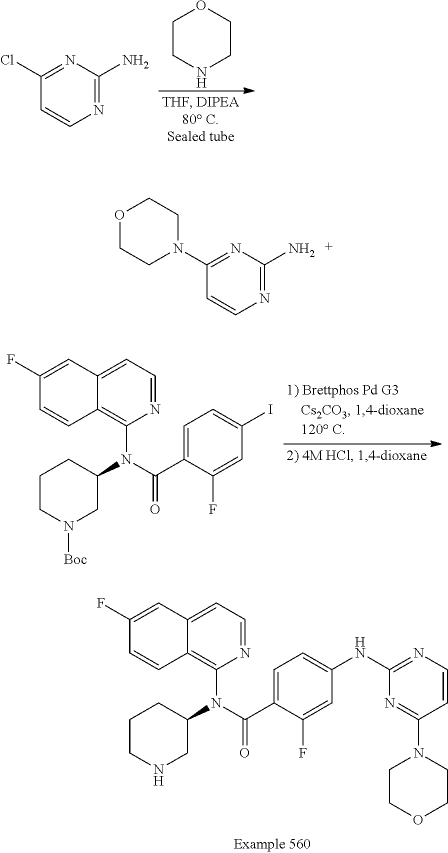

### Step 1:

To a solution of 4-chloropyrimidin-2-amine (300 mg, 2.316 mmol) in MeCN (5 mL) were added morpholine (404 mg, 4.63 mmol), DIPEA (1.213 mL, 6.95 mmol) at room temperature. The reaction mixture was stirred at 80° C. for 16 h in a sealed tube. The progress of the reaction was monitored by TLC. The reaction mixture was diluted with water and extracted with EtOAc (2×50 mL). The organic layers were washed with brine dried over Na₂SO₄ and concentrated under reduced pressure to afford 4-morpholinopyrimidin-2-amine (310 mg, 72.8%) as a pale yellow solid. LC-MS: [M+H⁺]=181.25.

### Steps 2-3:

Example 560 was prepared according to the procedure for Example 558 by substituting 4-(pyridin-4-yl)pyrimidin-2-amine with 4-morpholinopyrimidin-2-amine, provided the title compound hydrochloride salt as an off-white solid. LC-MS: [M+H⁺]=546.2. ¹H NMR (CD₃OD, 400 MHZ): δ 8.45-8.40 (m, 1H), 8.23-8.20 (m, 1H), 7.79-7.75 (m, 2H), 7.62-7.49 (m, 2H), 7.12-7.02 (m, 2H), 6.88-6.86 (m, 1H), 6.58 (d, J=7.6 Hz, 1H), 5.26-5.20 (m, 1H), 3.92-3.89 (m, 1H), 3.75-3.65 (m, 10H) 2.88-2.81 (m, 1H), 1.98-1.82 (m, 3H), 1.39-1.29 (m, 1H).

### Synthesis of (R)-2-fluoro-N-(6-fluoroisoquinolin-1-yl)-4-((4-(piperidin-1-yl)pyrimidin-2-yl)amino)-N-(piperidin-3-yl)benzamide (Example 561)

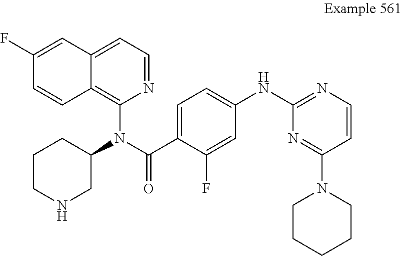 [SMILES: O=C(c1ccc(Nc2nccc(N3CCCCC3)n2)cc1F)N(c1nccc2cc(F)ccc12)[C@@H]1CCCNC1]

Example 561 was prepared according to the procedure described for Example 560 by substituting 4-morpholinopyrimidin-2-amine with 4-(piperidin-1-yl)pyrimidin-2-amine, provided the title compound hydrochloride salt as an off-white solid. LC-MS: [M+H⁺]=544.3. HPLC: 98.12%. ¹H NMR (CD₃OD, 400 MHZ): δ 8.43 (d, J=5.6 Hz, 1H), 8.23-8.19 (m, 1H), 7.76-7.70 (m, 2H), 7.61-7.48 (m, 2H), 7.15-7.12 (m, 1H), 7.04-7.00 (m, 1H), 6.85-6.83 (m, 1H), 6.57 (d, J=7.6 Hz, 1H), 5.27-5.20 (m, 1H), 3.92-3.89 (m, 1H), 3.74-3.66 (m, 6H), 3.35-3.29 (m, 1H), 2.88-2.81 (m, 1H), 1.79-1.63 (m, 7H), 1.31-1.24 (m, 1H).

### Synthesis of (R)-2-fluoro-N-(6-fluoroisoquinolin-1-yl)-4-((4-(4-methylpiperazin-1-yl)pyrimidin-2-yl)amino)-N-(piperidin-3-yl)benzamide (Example 562)

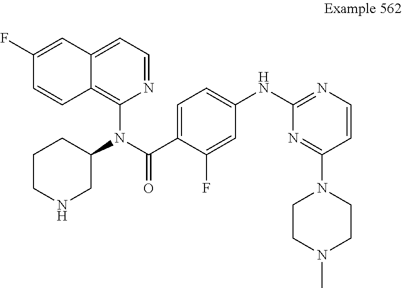 [SMILES: CN1CCN(c2ccnc(Nc3ccc(C(=O)N(c4nccc5cc(F)ccc45)[C@@H]4CCCNC4)c(F)c3)n2)CC1]

Example 562 was prepared according to the procedure described for Example 560 by substituting 4-morpholinopyrimidin-2-amine with 4-(4-methylpiperazin-1-yl)pyrimidin-2-amine, provided the title compound hydrochloride salt as a light brown solid. LC-MS: [M+H⁺]=559.3. HPLC: 95.1%. ¹H NMR (CD₃OD, 400 MHZ): δ 8.46-8.45 (m, 1H), 8.23-8.19 (m, 1H), 7.90-7.89 (m, 1H), 7.81-7.76 (m, 1H), 7.59-7.52 (m, 2H), 7.10-7.02 (m, 2H), 6.92-6.86 (m, 1H), 671-6.70 (m, 1H), 5.27-5.22 (m, 1H), 3.92-3.89 (m, 1H), 3.75-3.65 (m, 5H), 2.99 (s, 3H), 2.87-2.84 (m, 1H), 199-1.89 (m, 3H), 1.31-1.22 (m, 1H).

### Synthesis of tert-butyl (3R)-3-[[2-fluoro-4-(1-methyltriazol-4-yl)benzoyl]-[2-(pyridine-3-carbonyl)thieno[3,2-c]pyridin-4-yl]amino]piperidine-1-carboxylate (Example 563)

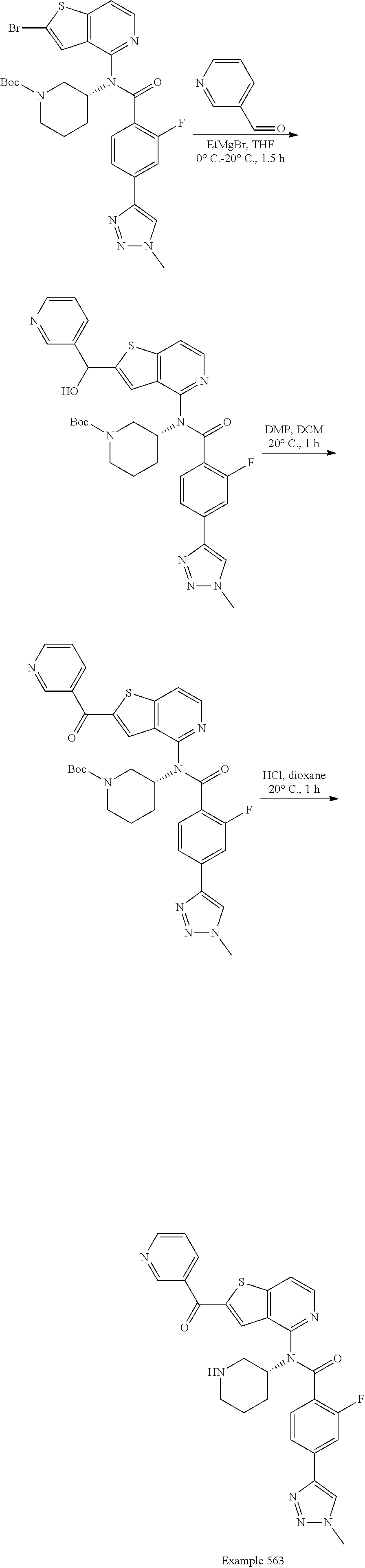

### Step 1:

To a solution of tert-butyl (3R)-3-[(2-bromothieno[3,2-c]pyridin-4-yl)-[2-fluoro-4-(1-methyltriazol-4-yl)benzoyl]amino]piperidine-1-carboxylate (300 mg, 438.66 μmol) in THF (9 mL) was cooled to 0° C. under N₂, then bromo (ethyl) magnesium (3 M, 292.44 μL) was dropwise added at 0° C. under N₂ atmosphere, and the resulting mixture was stirred at 0° C. for 30 min. Then a solution of pyridine-3-carbaldehyde (93.97 mg, 877.31 μmol) was dropwise added into the above mixture at 0° C. under N₂ atmosphere, the mixture was stirred at 20° C. for 1 h. The reaction mixture was slowly quenched with aq. NH₄Cl solution (5 mL) at 0° C., and then extracted with ethyl acetate (3×15 mL). The combined organic layers were washed with brine (3×5 mL), dried over Na₂SO₄, filtered and concentrated under reduced pressure to give crude tert-butyl (3R)-3-[[2-fluoro-4-(1-methyltriazol-4-yl)benzoyl]-[2-[hydroxy (3-pyridyl)methyl]thieno[3,2-c]pyridin-4-yl]amino]piperidine-1-carboxylate (200 mg, crude) as a yellow solid, which was used for next step directly without purification.

### Step 2:

To a solution of tert-butyl (3R)-3-[[2-fluoro-4-(1-methyltriazol-4-yl)benzoyl]-[2-[hydroxy (3-pyridyl)methyl]thieno[3,2-c]pyridin-4-yl]amino]piperidine-1-carboxylate (100 mg, 155.34 μmol, 1 eq) in DCM (2 mL) was added DMP (98.83 mg, 233.02 μmol). The mixture was stirred at 20° C. for 2 h. The reaction mixture was quenched with water (5 mL) at 0° C., and then diluted with (5 mL) and extracted with EtOAc (3×5 mL). The combined organic layers were washed with brine (3×5 mL), dried over Na₂SO₄, filtered and concentrated to give crude tert-butyl (3R)-3-[[2-fluoro-4-(1-methyltriazol-4-yl)benzoyl]-[2-(pyridine-3-carbonyl)thieno[3,2-c]pyridin-4-yl]amino]piperidine-1-carboxylate (60 mg, crude) as a yellow solid.

### Step 3:

The title compound hydrochloride salt was obtained as a white solid by deprotection Method A used in Example 97. LC-MS: [M+H⁺]=542.3. ¹H NMR (400 MHZ, DMSO-d₆): δ 9.35 (br s, 1H), 9.14-8.97 (m, 3H), 8.58-8.43 (m, 2H), 8.34 (br d, J=6.3 Hz, 1H), 8.29-8.13 (m, 2H), 7.85 (br d, J=5.0 Hz, 1H), 7.52-7.37 (m, 2H), 7.23-7.08 (m, 1H), 5.12-4.86 (m, 1H), 4.03 (br s, 3H), 3.70-3.53 (m, 1H), 3.38-3.24 (m, 1H), 3.15 (br d, J=11.1 Hz, 1H), 2.73-2.57 (m, 1H), 1.97-1.69 (m, 3H), 1.16-0.96 (m, 1H).

### Synthesis of (R)-2-fluoro-N-(6-fluoro-8-methylisoquinolin-1-yl)-N-(piperidin-3-yl)-4-(pyrimidin-2-ylamino)benzamide (Example 564)

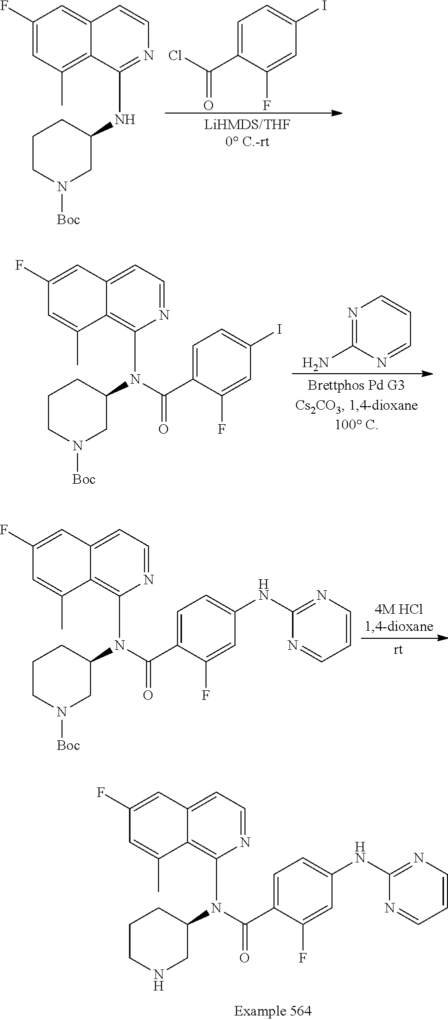

### Step 1:

To a stirred solution of tert-butyl (R)-3-((6-fluoro-8-methylisoquinolin-1-yl)amino)piperidine-1-carboxylate (550 mg, 1.530 mmol), DMAP (37.4 mg, 0.306 mmol) and DIPEA (1.604 mL, 9.18 mmol) in THF (10 mL) was added 2-fluoro-4-iodobenzoyl chloride (871 mg, 3.06 mmol) at room temperature and stirred for 16 h. The reaction mixture was diluted with water (10 mL) and extracted with EtOAc (2×30 mL). The combined organic layer was washed with brine, dried over Na₂SO₄, and concentrated under reduced pressure. The residue was purified by combi-flash using 20% EtOAc in hexane to afford tert-butyl (R)-3-(2-fluoro-N-(6-fluoro-8-methylisoquinolin-1-yl)-4-iodobenzamido) piperidine-1-carboxylate (650 mg, 68.8%) as a light brown solid. LC-MS: [M+H⁺]=608.2.

### Step 2:

To a stirred solution of tert-butyl (R)-3-(2-fluoro-N-(6-fluoro-8-methylisoquinolin-1-yl)-4-iodobenzamido) piperidine-1-carboxylate (250 mg, 0.412 mmol), pyrimidine-2-amine (78 mg, 0.823 mmol) and Cs₂CO₃ (402 mg, 1.235 mmol) in THF (2 mL), purged with nitrogen for 10 min. Then, Brettphos Pd G3 (37.3 mg, 0.041 mmol) was added to reaction mixture and stirred at 100° C. for 16 h. The reaction mixture was diluted with EtOAc (50 mL), filtered through celite bed and filtrate was concentrated under reduced pressure. The residue was purified by Combi-flash using 30% EtOAc in hexane to afford tert-butyl (R)-3-(2-fluoro-N-(6-fluoro-8-methylisoquinolin-1-yl)-4-(pyrimidin-2-ylamino) benzamido) piperidine-1-carboxylate (110 mg, 46.5%) as a brown solid. LC-MS: [M+H⁺]=575.3.

### Step 3:

The title compound hydrochloride salt was obtained as an off-white solid by deprotection Method A used in Example 97. LC-MS: [M+H⁺]=475.2. ¹H NMR (CD₃OD, 400 MHZ): δ 8.55 (d, J=4.8 Hz, 2H), 8.44-8.40 (m, 1H), 7.73-7.68 (m, 1H), 7.45-7.42 (m, 1H), 7.37-7.36 (m, 1H), 7.34-7.28 (m, 1H), 7.02-7.00 (m, 1H), 6.91-6.88 (m, 1H), 6.76-6.72 (m, 1H), 5.10-5.03 (m, 1H), 3.97-3.93 (m, 1H), 3.84-3.79 (m, 1H), 3.41-3.34 (m, 1H), 3.04-2.96 (m, 1H), 2.82 (s, 3H), 2.71-2.67 (m, 1H), 1.96-1.88 (m, 2H), 1.61-1.57 (m, 1H).

### Synthesis of N-[2-(4-amino-3,5-difluoro-phenyl)thieno[3,2-c]pyridin-4-yl]-2-fluoro-N-[(3R)-3-piperidyl]-4-(triazolo[4,5-b]pyridin-3-yl)benzamide (Example 565)

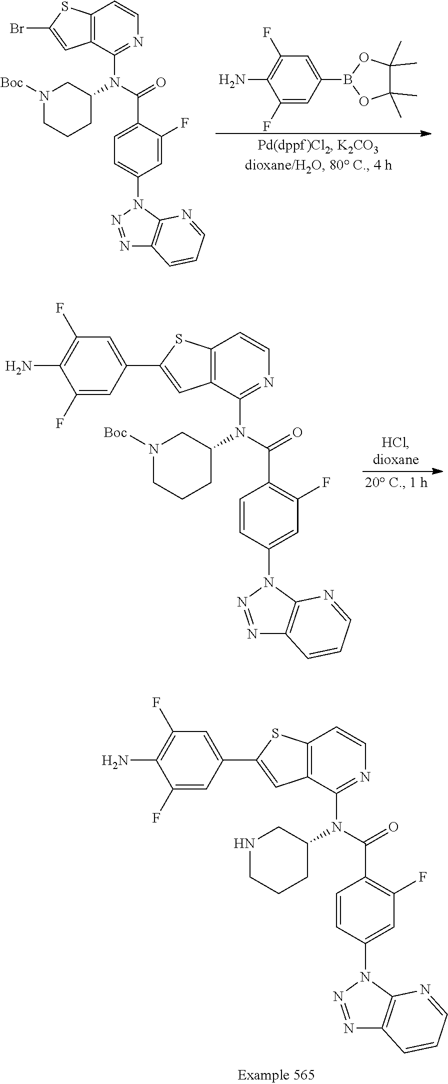 [SMILES: CC(C)(C)OC(=O)N1CCC[C@@H](N(C(=O)c2ccc(-n3nnc4cccnc43)cc2F)c2nccc3sc(Br)cc23)C1 ; CC1(C)OB(c2cc(F)c(N)c(F)c2)OC1(C)C ; CC(C)(C)OC(=O)N1CCC[C@@H](N(C(=O)c2ccc(-n3nnc4cccnc43)cc2F)c2nccc3sc(-c4cc(F)c(N)c(F)c4)cc23)C1 ; Nc1c(F)cc(-c2cc3c(N(C(=O)c4ccc(-n5nnc6cccnc65)cc4F)[C@@H]4CCCNC4)nccc3s2)cc1F]

Example 565 was prepared according to the procedure described for Example 161 by substituting 4-pyridylboronic acid with 2,6-difluoro-4-(4,4,5,5-tetramethyl-1,3,2-dioxaborolan-2-yl)aniline, provided the title compound hydrochloride salt as an off-white solid. LC-MS: [M+H⁺]=601.3. ¹H NMR (400 MHZ, CD₃OD): δ 8.75 (br d, J=3.6 Hz, 1H), 8.50 (d, J=8.3 Hz, 1H), 8.33 (d, J=5.4 Hz, 1H), 8.12 (br d, J=11.1 Hz, 1H), 7.99 (br d, J=7.8 Hz, 1H), 7.82 (d, J=5.4 Hz, 1H), 7.64 (s, 1H), 7.53 (dd, J=4.4, 8.3 Hz, 1H), 7.41 (br t, J=7.8 Hz, 1H), 7.35-7.27 (m, 2H), 5.30-5.19 (m, 1H), 3.96-3.83 (m, 1H), 3.71-3.61 (m, 1H), 3.36 (br d, J=1.3 Hz, 1H), 2.97-2.82 (m, 1H), 2.11 (br dd, J=2.0, 9.4 Hz, 1H), 2.00-1.84 (m, 2H), 1.46-1.31 (m, 1H).

### Synthesis of 2-fluoro-N-[2-(2-fluoro-4-pyridyl)thieno [3, 2-c] pyridin-4-yl]-N-[(3R)-3-piperidyl]-4-(triazolo [4, 5-b] pyridin-3-yl) benzamide (Example 566)

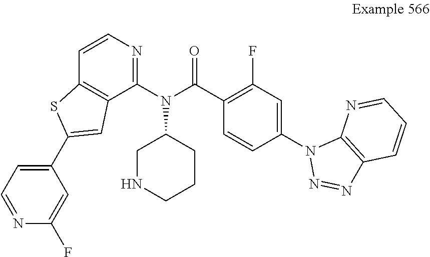 [SMILES: O=C(c1ccc(-n2nnc3cccnc32)cc1F)N(c1nccc2sc(-c3ccnc(F)c3)cc12)[C@@H]1CCCNC1]

Example 566 was prepared according to the procedure described for Example 161 by substituting 4-pyridylboronic acid with 2-fluoro-4-(4, 4, 5, 5-tetramethyl-1, 3, 2-dioxaborolan-2-yl) pyridine, provided the title compound hydrochloride salt (44%) as a white solid. LC-MS: [M+H⁺]=569.3. ¹H NMR (400 MHZ, CD₃OD): δ 8.74 (br d, J=3.9 Hz, 1H), 8.55-8.41 (m, 2H), 8.33 (d, J=5.3 Hz, 1H), 8.20 (s, 1H), 8.08 (br d, J=11.1 Hz, 1H), 8.04-7.88 (m, 2H), 7.71 (br d, J=4.6 Hz, 1H), 7.60-7.37 (m, 3H), 5.41-5.15 (m, 1H), 3.94 (br d, J=10.6 Hz, 1H), 3.70 (br t, J=11.4 Hz, 1H), 3.40-3.33 (m, 1H), 2.99-2.83 (m, 1H), 2.12 (br d, J=9.4 Hz, 1H), 2.03-1.84 (m, 2H), 1.50-1.29 (m, 1H).

### Synthesis of 2-fluoro-4-[4-[[2-fluoro-4-(triazolo[4,5-b]pyridin-3-yl)benzoyl]-[(3R)-3-piperidyl]amino] thieno[3,2-c]pyridin-2-yl]benzamide (Example 567)

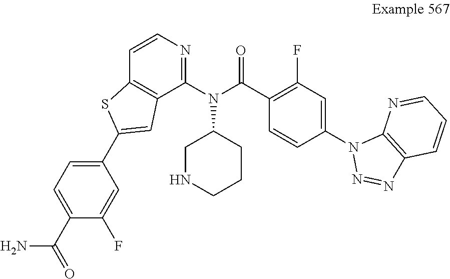 [SMILES: NC(=O)c1ccc(-c2cc3c(N(C(=O)c4ccc(-n5nnc6cccnc65)cc4F)[C@@H]4CCCNC4)nccc3s2)cc1F]

Example 567 was prepared according to the procedure described for Example 161 by substituting 4-pyridylboronic acid with 2-fluoro-4-(4,4,5,5-tetramethyl-1,3,2-dioxaborolan-2-yl)benzamide, provided the title compound hydrochloride salt as a yellow solid. LC-MS: [M+H⁺]=611.3. ¹H NMR (400 MHZ, CD₃OD): δ 8.73 (br d, J=4.1 Hz, 1H), 8.49 (br d, J=8.0 Hz, 1H), 8.42 (br d, J=5.4 Hz, 1H), 8.08 (br d, J=11.2 Hz, 1H), 8.03-7.88 (m, 4H), 7.76-7.64 (m, 2H), 7.57-7.39 (m, 2H), 5.35-5.20 (m, 1H), 4.01-3.85 (m, 1H), 3.78-3.63 (m, 1H), 3.41-3.34 (m, 1H), 2.98-2.82 (m, 1H), 2.12 (br dd, J=2.2, 8.1 Hz, 1H), 2.02-1.85 (m, 2H), 1.51-1.26 (m, 1H).

### Synthesis of 4-[4-[[2-fluoro-4-(triazolo[4,5-b]pyridin-3-yl)benzoyl]-[(3R)-3-piperidyl]amino]thieno[3,2-c]pyridin-2-yl]thiophene-2-carboxamide (Example 568)

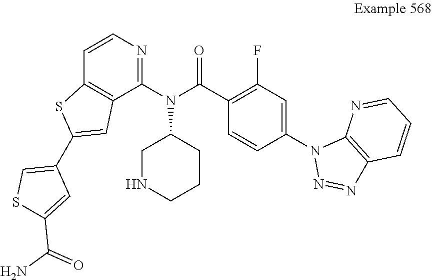 [SMILES: NC(=O)c1cc(-c2cc3c(N(C(=O)c4ccc(-n5nnc6cccnc65)cc4F)[C@@H]4CCCNC4)nccc3s2)cs1]

Example 568 was prepared according to the procedure described for Example 161 by substituting 4-pyridylboronic acid with 4-(4,4,5,5-tetramethyl-1,3,2-dioxaborolan-2-yl)thiophene-2-carboxamide, provided the title compound hydrochloride salt as a yellow solid. LC-MS: [M+H⁺]=599.3. ¹H NMR (400 MHz, CD₃OD): δ 8.74 (br d, J=3.8 Hz, 1H), 8.50 (br d, J=8.4 Hz, 1H), 8.37 (br d, J=5.4 Hz, 1H), 8.21-7.92 (m, 4H), 7.87 (br d, J=5.4 Hz, 1H), 7.72 (s, 1H), 7.56-7.41 (m, 2H), 5.36-5.17 (m, 1H), 4.00-3.82 (m, 1H), 3.65 (br t, J=12.0 Hz, 1H), 3.36 (br d, J=12.9 Hz, 1H), 2.98-2.80 (m, 1H), 2.52-2.23 (m, 1H), 2.12 (br d, J=11.0 Hz, 1H), 2.04-1.85 (m, 2H), 1.50-1.34 (m, 1H).

### Synthesis of [4-[4-[[2-fluoro-4-(triazolo[4,5-b]pyridin-3-yl)benzoyl]-[(3R)-3-piperidyl]amino]thieno[3,2-c]pyridin-2-yl]phenyl]boronic acid (Example 569)

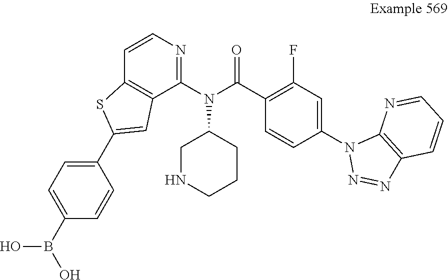 [SMILES: O=C(c1ccc(-n2nnc3cccnc32)cc1F)N(c1nccc2sc(-c3ccc(B(O)O)cc3)cc12)[C@@H]1CCCNC1]

Example 569 was prepared according to the procedure described for Example 161 by substituting 4-pyridylboronic acid with (4-boronophenyl)boronic acid, provided the title compound hydrochloride salt as a yellow solid. LC-MS: [M+H⁺]=594.3. ¹H NMR (400 MHz, CD₃OD): δ 8.73 (br d, J=4.0 Hz, 1H), 8.49 (br d, J=8.1 Hz, 1H), 8.37 (br d, J=5.4 Hz, 1H), 8.11 (br d, J=10.3 Hz, 1H), 7.99 (br d, J=7.9 Hz, 1H), 7.93-7.71 (m, 5H), 7.52 (br dd, J=4.5, 8.3 Hz, 1H), 7.42 (br t, J=7.7 Hz, 1H), 5.31-5.22 (m, 1H), 3.98-3.83 (m, 1H), 3.76-3.62 (m, 2H), 2.88 (dt, J=3.5, 12.1 Hz, 1H), 2.20-2.06 (m, 1H), 2.01-1.84 (m, 2H), 1.49-1.25 (m, 2H).

### Synthesis of N-[2-[4-(2-amino-2-oxo-ethyl)phenyl]thieno[3,2-c]pyridin-4-yl]-2-fluoro-N-[(3R)-3-piperidyl]-4-(triazolo[4,5-b]pyridin-3-yl)benzamide (Example 570)

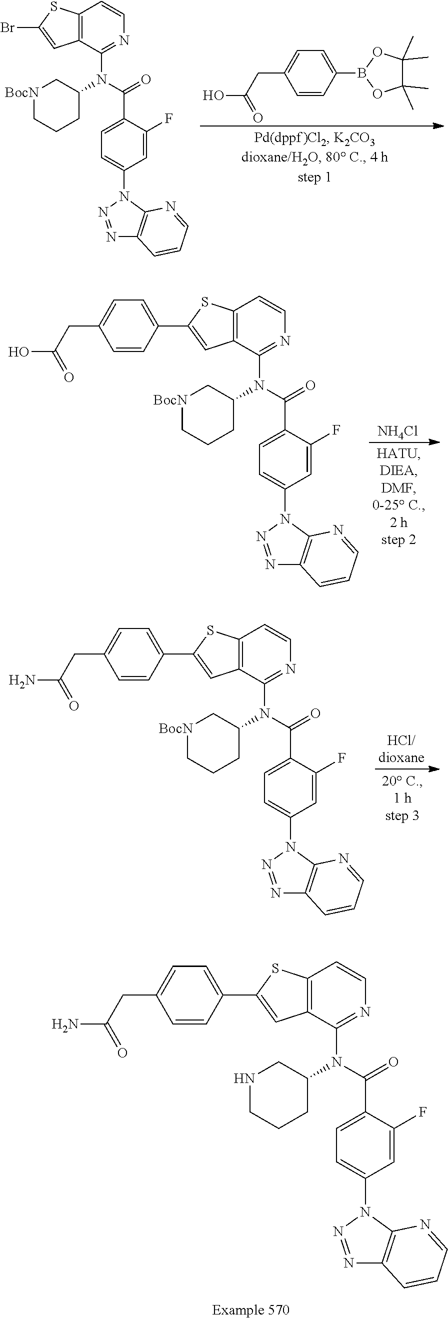 [SMILES: CC(C)(C)OC(=O)N1CCC[C@@H](N(C(=O)c2ccc(-n3nnc4cccnc43)cc2F)c2nccc3sc(Br)cc23)C1 ; CC1(C)OB(c2ccc(CC(=O)O)cc2)OC1(C)C ; CC(C)(C)OC(=O)N1CCC[C@@H](N(C(=O)c2ccc(-n3nnc4cccnc43)cc2F)c2nccc3sc(-c4ccc(CC(=O)O)cc4)cc23)C1 ; "NH4Cl" ; CC(C)(C)OC(=O)N1CCC[C@@H](N(C(=O)c2ccc(-n3nnc4cccnc43)cc2F)c2nccc3sc(-c4ccc(CC(N)=O)cc4)cc23)C1 ; NC(=O)Cc1ccc(-c2cc3c(N(C(=O)c4ccc(-n5nnc6cccnc65)cc4F)[C@@H]4CCCNC4)nccc3s2)cc1]

### Step 1:

2-[4-[4-[[(3R)-1-tert-butoxycarbonyl-3-piperidyl]-[2-fluoro-4-(triazolo[4,5-b]pyridin-3-yl)benzoyl]amino]thieno[3,2-c]pyridin-2-yl] phenyl]acetic acid was prepared according to Example 161 by substituting 4-pyridylboronic acid with 2-[4-(4,4,5,5-tetramethyl-1,3,2-dioxaborolan-2-yl)phenyl]acetic acid.

### Step 2:

To a solution of 2-[4-[4-[[(3R)-1-tert-butoxycarbonyl-3-piperidyl]-[2-fluoro-4-(triazolo [4,5-b]pyridin-3-yl)benzoyl]amino]thieno[3,2-c]pyridin-2-yl]phenyl]acetic acid (100 mg, 141.29 μmol) in THF (1 mL) was added NH₄Cl (22.67 mg, 423.87 μmol), HATU (64.47 mg, 169.55 μmol) and DIEA (54.78 mg, 423.87 μmol). The mixture was stirred at 25° C. for 4 h. The reaction mixture was filtered and concentrated under reduced pressure to give a residue. The residue was purified by prep-TLC (SiO₂, Petroleum ether/Ethyl acetate=0/1) to give tert-butyl (3R)-3-[[2-[4-(2-amino-2-oxo-ethyl)phenyl]thieno[3,2-c]pyridin-4-yl]-[2-fluoro-4-(triazolo[4,5-b]pyridin-3-yl)benzoyl]amino]piperidine-1-carboxylate (60 mg, 60%) as a yellow solid.

### Step 3:

The title compound hydrochloride salt was obtained as an off-white solid by deprotection Method A used in Example 97. LC-MS: [M+H⁺]=607.4. ¹H NMR (400 MHZ, CD₃OD): δ 8.74 (br d, J=4.0 Hz, 1H), 8.50 (br d, J=8.3 Hz, 1H), 8.37 (d, J=5.5 Hz, 1H), 8.10 (br d, J=10.5 Hz, 1H), 7.99 (br d, J=9.3 Hz, 1H), 7.87 (d, J=5.4 Hz, 1H), 7.80-7.70 (m, 3H), 7.52 (dd, J=4.4, 8.3 Hz, 1H), 7.49-7.37 (m, 3H), 5.33-5.21 (m, 1H), 3.98-3.83 (m, 1H), 3.73-3.62 (m, 1H), 3.37 (br s, 2H), 2.95-2.84 (m, 1H), 2.54-2.18 (m, 1H), 2.17-2.06 (m, 1H), 2.00-1.86 (m, 2H), 1.48-1.27 (m, 1H).

### Synthesis of N-[2-(2,6-dimethyl-4-pyridyl)thieno[3,2-c]pyridin-4-yl]-2-fluoro-N-[(3R)-3-piperidyl]-4-(triazolo[4,5-b] pyridin-3-yl)benzamide (Example 571)

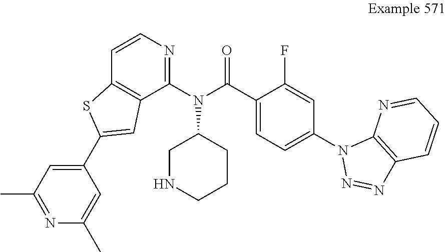 [SMILES: Cc1cc(-c2cc3c(N(C(=O)c4ccc(-n5nnc6cccnc65)cc4F)[C@@H]4CCCNC4)nccc3s2)cc(C)n1]

Example 571 was prepared according to the procedure described for Example 161 by substituting 4-pyridylboronic acid with 2,6-dimethyl-4-(4,4,5,5-tetramethyl-1,3,2-dioxaborolan-2-yl)pyridine, provided the title compound hydrochloride salt as a white solid. LC-MS: [M+H+]=579.4. ¹H NMR (400 MHZ, CD₃OD): δ 8.73 (br d, J=2.9 Hz, 1H), 8.51 (br d, J=6.4 Hz, 3H), 8.16 (br s, 2H), 8.09-7.93 (m, 3H), 7.59-7.43 (m, 2H), 5.39-5.22 (m, 1H), 3.97 (br d, J=9.9 Hz, 1H), 3.72 (br t, J=11.3 Hz, 1H), 3.38 (br d, J=12.3 Hz, 1H), 3.00-2.77 (m, 7H), 2.23-2.07 (m, 1H), 1.97 (br s, 2H), 1.44 (br d, J=6.3 Hz, 1H).

### Synthesis of 4-[4-[[2-fluoro-4-(triazolo [4, 5-b] pyridin-3-yl) benzoyl]-[(3R)-3-piperidyl] amino] thieno [3, 2-c] pyridin-2-yl]-2-methyl-benzamide (Example 572)

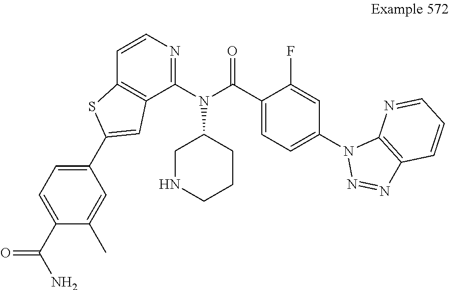 [SMILES: Cc1cc(-c2cc3c(N(C(=O)c4ccc(-n5nnc6cccnc65)cc4F)[C@@H]4CCCNC4)nccc3s2)ccc1C(N)=O]

Example 572 was prepared according to the procedure described for Example 161 by substituting 4-pyridylboronic acid with 2-methyl-4-(4, 4, 5, 5-tetramethyl-1, 3, 2-dioxaborolan-2-yl) benzoic acid, provided the title compound hydrochloride salt as a white solid. LC-MS: [M+H⁺]=607.4. ¹H NMR (400 MHZ, CD₃OD): δ 8.73 (br d, J=3.9 Hz, 1H), 8.49 (br d, J=8.1 Hz, 1H), 8.39 (d, J=5.5 Hz, 1H), 8.09 (br d, J=10.8 Hz, 1H), 7.99 (br d, J=8.3 Hz, 1H), 7.91-7.81 (m, 2H), 7.75-7.62 (m, 2H), 7.60-7.49 (m, 2H), 7.41 (br t, J=7.6 Hz, 1H), 5.37-5.21 (m, 1H), 3.93 (br d, J=10.1 Hz, 1H), 3.76-3.63 (m, 1H), 3.36 (br d, J=12.1 Hz, 1H), 2.97-2.83 (m, 1H), 2.53 (s, 3H), 2.11 (br d, J=8.9 Hz, 1H), 2.03-1.83 (m, 2H), 1.51-1.31 (m, 1H).

### Synthesis of 4-[4-[[2-fluoro-4-(triazolo [4, 5-b] pyridin-3-yl) benzoyl]-[(3R)-3-piperidyl] amino] thieno [3, 2-c] pyridin-2-yl]-2-(trifluoromethyl) benzamide (Example 573)

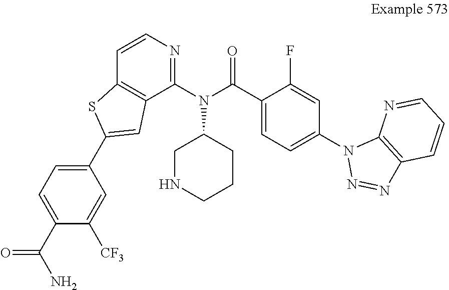 [SMILES: NC(=O)c1ccc(-c2cc3c(N(C(=O)c4ccc(-n5nnc6cccnc65)cc4F)[C@@H]4CCCNC4)nccc3s2)cc1C(F)(F)F]

Example 573 was prepared according to the procedure described for Example 161 by substituting 4-pyridylboronic acid with 4-(4,4,5,5-tetramethyl-1,3,2-dioxaborolan-2-yl)-2-(trifluoromethyl)benzoic acid, provided the title compound hydrochloride salt as a white solid. LC-MS: [M+H⁺]=661.3. ¹H NMR (400 MHZ, CD₃OD): δ 8.73 (br d, J=3.9 Hz, 1H), 8.50 (br d, J=8.4 Hz, 1H), 8.43 (d, J=5.4 Hz, 1H), 8.15-8.05 (m, 3H), 8.04-7.89 (m, 3H), 7.72 (br d, J=8.0 Hz, 1H), 7.52 (dd, J=4.5, 8.3 Hz, 1H), 7.41 (br t, J=7.9 Hz, 1H), 5.36-5.18 (m, 1H), 3.94 (br d, J=11.0 Hz, 1H), 3.76-3.63 (m, 1H), 3.39-3.33 (m, 1H), 3.00-2.80 (m, 1H), 2.12 (br d, J=11.6 Hz, 1H), 2.03-1.81 (m, 2H), 1.51-1.29 (m, 1H).

### Synthesis of (R)-4-(3H-[1,2,3]triazolo[4,5-b]pyridin-3-yl)-2-fluoro-N-(6-(5-methylthiazol-2-yl)isoquinolin-1-yl)-N-(piperidin-3-yl)benzamide (Example 574)

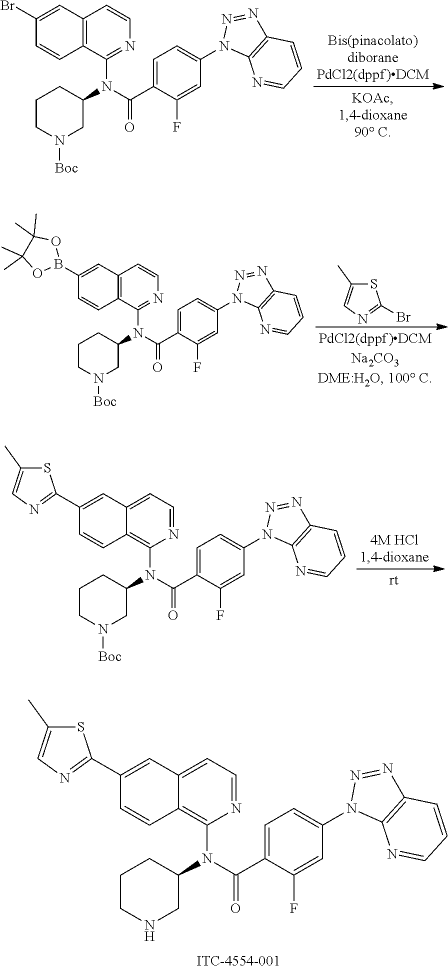

### Step 1:

A mixture of tert-butyl (R)-3-(4-(3H-[1,2,3]triazolo[4,5-b]pyridin-3-yl)-2-fluoro-N-(6-(4,4,5,5-tetramethyl-1,3,2-dioxaborolan-2-yl)isoquinolin-1-yl)benzamido)piperidine-1-carboxylate (500 mg, 0.773 mmol), bis(pinacolato) diborane (589 mg, 2.320 mmol) and potassium acetate (228 mg, 2.320 mmol) was dissolved in 1,4-dioxane (10 mL) at room temperature. The reaction mixture was degassed with nitrogen for 10 min and followed by addition of PdCl₂(dppf)-CH₂Cl₂ adduct (31.6 mg, 0.039 mmol). The reaction mixture was stirred at 90° C. for 16 h before it was diluted with water (50 mL) and extracted with EtOAc (2×10 mL). The combined organic layer was dried over Na₂SO₄ and concentrated under reduced pressure. The residue was purified by Combi-flash eluting with 30-35% EtOAc in hexane to afford tert-butyl (R)-3-(4-(3H-[1,2,3]triazolo[4,5-b]pyridin-3-yl)-2-fluoro-N-(6-(4,4,5,5-tetramethyl-1,3,2-dioxaborolan-2-yl) isoquinolin-1-yl) benzamido) piperidine-1-carboxylate (300 mg, 52.6%) as a yellow solid. LC-MS: [M+H⁺]=694.3.

### Step 2:

A mixture of tert-butyl (R)-3-(4-(3H-[1,2,3]triazolo[4,5-b]pyridin-3-yl)-2-fluoro-N-(6-(4,4,5,5-tetramethyl-1,3,2-dioxaborolan-2-yl) isoquinolin-1-yl) benzamido) piperidine-1-carboxylate (50 mg, 0.072 mmol), 2-bromo-5-methylthiazole (15.4 mg, 0.087 mmol) and sodium carbonate (22.92 mg, 0.216 mmol) were dissolved in DME (1 mL) and water (1 mL) at room temperature. The reaction mixture was degassed with nitrogen for 10 min and followed by addition of PdCl₂(dppf)-CH₂Cl₂ adduct (2.94 mg, 3.60 mmol). The reaction mixture and stirred at 100° C. for 16 h before it was diluted with water (30 mL) and extracted with EtOAc (2×50 mL). The combined organic layer was dried over Na₂SO₄ and concentrated under reduced pressure. The residue was purified by combi-flash and eluted with 50-60% EtOAc in hexane and was further purified by prep HPLC (Conditions: Kinetex evo C-18 (250×21.2×5 μm), Mobile phase A: 0.1% formic acid in water (100%), Mobile phase B: 100% MeCN, Gradient: Time (min)/% B: 0/10, 6/60, 10/95, Flow: 15 mL/min, Dilution: MeCN+H₂O). Pure fractions were combined and concentrated under reduced pressure to afford tert-butyl (R)-3-(4-(3H-[1,2,3]triazolo[4,5-b]pyridin-3-yl)-2-fluoro-N-(6-(5-methylthiazol-2-yl)isoquinolin-1-yl)benzamido)piperidine-1-carboxylateformate salt (20 mg, 36.9%) as an off-white solid. LC-MS: [M+H⁺]=665.45.

### Step 3:

The title compound hydrochloride salt was obtained as an off-white solid by deprotection Method A used in Example 97. LC-MS: [M+H⁺]=565.2. ¹H NMR (CD₃OD, 400 MHZ): δ8.71 (d, J=2.8 Hz, 1H), 8.51-8.46 (m, 2H), 8.34-8.26 (m, 3H), 8.06-8.03 (m, 1H), 7.93-7.91 (m, 1H), 7.85 (d, J=5.6 Hz, 1H), 7.64-7.60 (m, 1H), 7.51-7.48 (m, 1H), 7.36-7.32 (m, 1H), 5.32-5.28 (m, 1H), 4.22-4.20 (m, 1H), 3.99-3.96 (m, 1H), 3.74-3.68 (m, 1H), 2.99-2.82 (m, 1H), 2.55 (s, 3H), 2.07-1.91 (m, 3H), 1.44-1.34 (m, 1H).

### Synthesis of (R)-4-(3H-[1,2,3]triazolo[4,5-b]pyridin-3-yl)-2-fluoro-N-(6-(2-methylthiazol-4-yl)isoquinolin-1-yl)-N-(piperidin-3-yl)benzamide (Example 575)

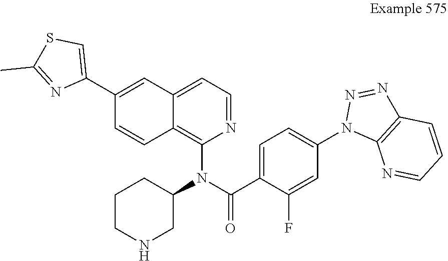 [SMILES: Cc1nc(-c2ccc3c(N(C(=O)c4ccc(-n5nnc6cccnc65)cc4F)[C@@H]4CCCNC4)nccc3c2)cs1]

Example 575 was synthesized following the procedure for Example 574 by substituting 2-bromo-5-methylthiazole with 4-bromo-2-methylthiazole, provided the title compound hydrochloride salt as an off-white solid. LC-MS: [M+H⁺]=565.2. 1H NMR (CD₃OD, 400 MHZ): δ 8.71-8.70 (m, 1H), 8.53-8.51 (m, 1H), 8.48-8.46 (m, 1H), 8.36 (m, 1H), 8.29-8.28 (m, 1H), 8.24-8.21 (m, 1H), 8.10-8.09 (m, 1H), 8.04-8.02 (m, 1H), 7.96-7.90 (m, 1H), 7.85-7.84 (m, 1H), 7.51-7.48 (m, 1H), 7.37-7.33 (m, 1H), 5.33-5.28 (m, 1H), 4.00-3.97 (m, 1H), 3.76-3.70 (m, 1H), 3.37-3.29 (m, 1H), 2.81 (s, 3H), 2.12-2.92 (m, 3H), 1.38-1.28 (m, 1H).

### Synthesis of (R)-4-(3H-[1,2,3]triazolo[4,5-b]pyridin-3-yl)-2-fluoro-N-(6-(5-methyl-1,3,4-thiadiazol-2-yl)isoquinolin-1-yl)-N-(piperidin-3-yl)benzamide (Example 576)

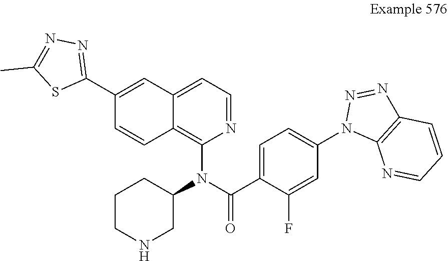 [SMILES: Cc1nnc(-c2ccc3c(N(C(=O)c4ccc(-n5nnc6cccnc65)cc4F)[C@@H]4CCCNC4)nccc3c2)s1]

Example 576 was synthesized following the procedure for Example 574 by substituting 2-bromo-5-methylthiazole with 2-bromo-5-methyl-1,3,4-thiadiazole, provided the title compound hydrochloride salt as an off-white solid. LC-MS: [M+H⁺]=566.25. ¹H NMR (CD₃OD, 400 MHZ): δ 8.71 (d, J=3.2 Hz, 1H), 8.56 (d, J=5.6 Hz, 1H), 8.48-8.46 (m, 1H), 8.43-8.34 (m, 3H), 8.05-8.03 (m, 1H), 7.93-7.89 (m, 2H), 7.72-7.61 (m, 1H), 7.52-7.50 (m, 1H), 7.37-7.33 (m, 1H), 5.33-5.30 (m, 1H), 4.22-4.20 (m, 1H), 4.00-3.97 (m, 1H), 3.75-3.63 (m, 1H), 3.39-3.33 (m, 1H), 2.82 (s, 3H), 2.07-1.91 (m, 3H), 1.45-1.28 (m, 1H).

### Synthesis of (R)-2-fluoro-4-(1-methyl-1H-1,2,3-triazol-4-yl)-N-(6-phenylisoquinolin-1-yl)-N-(piperidin-3-yl)benzamide (Example 577)

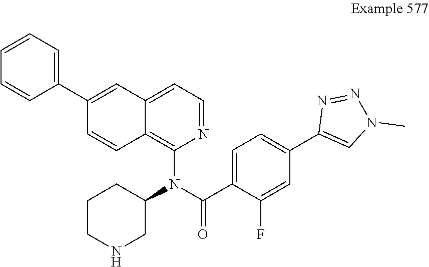 [SMILES: Cn1cc(-c2ccc(C(=O)N(c3nccc4cc(-c5ccccc5)ccc34)[C@@H]3CCCNC3)c(F)c2)nn1]

Example 577 was prepared according to the procedure described for Example 370 by substituting 3-pyridylboronic acid with (4-fluorophenyl) boronic acid with phenylboronic acid, provided the title compound hydrochloride salt as an off-white solid. LC-MS: [M+H⁺]=507.3. ¹H NMR (CD₃OD, 400 MHZ): δ 8.44 (d, J=5.6 Hz, 1H), 8.23-8.21 (m, 1H), 8.15 (bs, 1H), 8.11-8.02 (m, 2H), 7.84-7.82 (m, 1H), 7.76-7.70 (m, 2H), 7.51-7.40 (m, 3H), 7.30-7.13 (m, 3H), 5.29-5.23 (m, 1H), 4.04 (s, 3H), 3.96-3.93 (m, 1H), 3.72-3.65 (m, 1H), 3.35-3.20 (m, 1H), 2.90-2.83 (m, 1H), 2.05-1.89 (m, 3H), 1.39-1.28 (m, 1H).

### Synthesis of (R)-2-fluoro-N-(8-methylisoquinolin-1-yl)-4-((4-(piperidin-1-yl)pyrimidin-2-yl)amino)-N-(piperidin-3-yl)benzamide (Example 578)

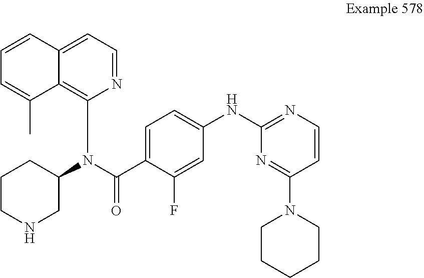 [SMILES: Cc1cccc2ccnc(N(C(=O)c3ccc(Nc4nccc(N5CCCCC5)n4)cc3F)[C@@H]3CCCNC3)c12]

Example 578 was prepared according to the procedure described for Example 558 by substituting 4-(pyridin-4-yl)pyrimidin-2-amine with 4-(piperidin-1-yl)pyrimidin-2-amine, provided the title compound hydrochloride salt as an off-white solid. LC-MS: [M+H⁺]=540.3. ¹H NMR (CD₃OD, 400 MHZ): δ 8.42-8.39 (m, 1H), 7.73-7.63 (m, 3H), 7.52-87.43 (m, 2H), 7.20-7.17 (m, 1H), 6.77-6.75 (m, 2H), 6.52-6.49 (m, 1H), 5.05-5.91 (m, 1H), 3.99-3.96 (m, 1H), 3.80-3.71 (m, 4H), 3.34-3.26 (m, 1H), 3.12-2.99 (m, 1H), 2.80 (s, 3H), 1.96-1.86 (m, 2H), 1.79-1.77 (m, 2H), 1.67-1.66 (m, 4H), 1.40-1.26 (m, 1H), 0.96-0.89 (m, 1H).

### Synthesis of (R)-2-fluoro-4-(1-methyl-1H-1,2,3-triazol-4-yl)-N-(6-phenylisoquinolin-1-yl)-N-(piperidin-3-yl)benzamide (Example 579)

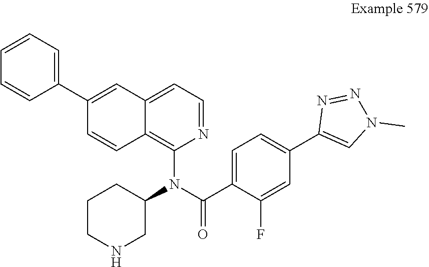 [SMILES: Cn1cc(-c2ccc(C(=O)N(c3nccc4cc(-c5ccccc5)ccc34)[C@@H]3CCCNC3)c(F)c2)nn1]

Example 579 was prepared according to the procedure for Example 370 by substituting 3-pyridylboronic acid with (4-fluorophenyl) boronic acid with phenylboronic acid, provided the title compound hydrochloride salt as an off-white solid. LC-MS: [M+H⁺]=507.3. HPLC: 99.37%. ¹H NMR (CD₃OD, 400 MHZ): δ 8.44 (d, J=5.6 Hz, 1H), 8.23-8.21 (m, 1H), 8.15 (bs, 1H), 8.11-8.02 (m, 2H), 7.84-7.82 (m, 1H), 7.76-7.70 (m, 2H), 7.51-7.40 (m, 3H), 7.30-7.13 (m, 3H), 5.29-5.23 (m, 1H), 4.04 (s, 3H), 3.96-3.93 (m, 1H), 3.72-3.65 (m, 1H), 3.35-3.20 (m, 1H), 2.90-2.83 (m, 1H), 2.05-1.89 (m, 3H), 1.39-1.28 (m, 1H).

### Synthesis of (R)-2-fluoro-N-(6-(4-fluorophenyl)isoquinolin-1-yl)-4-(1-methyl-1H-1,2,3-triazol-4-yl)-N-(piperidin-3-yl)benzamide (Example 580)

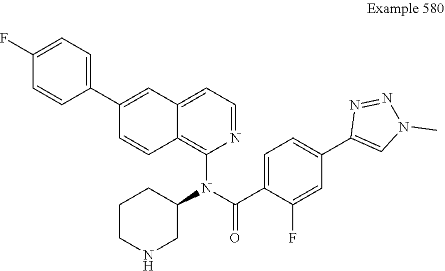 [SMILES: Cn1cc(-c2ccc(C(=O)N(c3nccc4cc(-c5ccc(F)cc5)ccc34)[C@@H]3CCCNC3)c(F)c2)nn1]

Example 580 was prepared according to the procedure described for Example 370 by substituting 3-pyridylboronic acid with (4-fluorophenyl) boronic acid, provided the title compound hydrochloride salt as an off-white solid. LC-MS: [M+H⁺]=525.3. ¹H NMR (CD₃OD, 400 MHZ): δ 8.45 (d, J=5.6 Hz, 1H), 8.27-8.15 (m, 2H), 8.10-8.00 (m, 2H), 7.90-7.75 (m, 3H), 7.30-7.14 (m, 5H), 5.29-5.23 (m, 1H), 4.05 (s, 3H), 3.97-3.94 (m, 1H), 3.72-3.66 (m, 1H), 3.36-3.30 (m, 1H), 2.90-2.83 (m, 1H), 2.05-1.85 (m, 3H), 1.39-1.28 (m, 1H).

### Synthesis of (R)-2-fluoro-4-(1-methyl-1H-1,2,3-triazol-4-yl)-N-(piperidin-3-yl)-N-(6-(pyridin-2-yl)isoquinolin-1-yl)benzamide (Example 581)

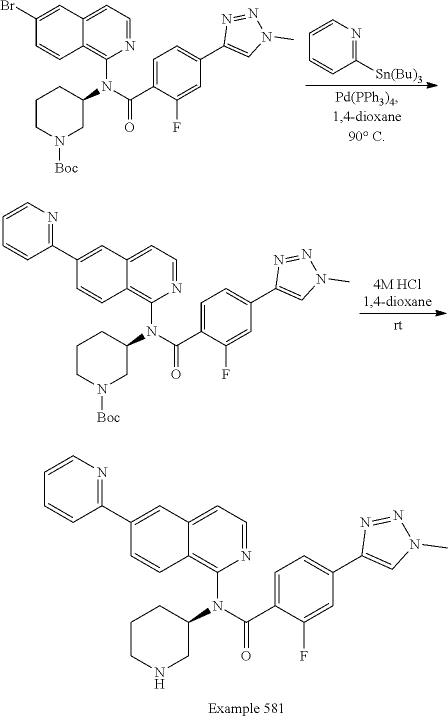

### Step 1:

A mixture of tert-butyl (R)-3-(N-(6-bromoisoquinolin-1-yl)-2-fluoro-4-(1-methyl-1H-1,2,3-triazol-4-yl)benzamido)piperidine-1-carboxylate (80 mg, 0.124 mmol) and 2-(tributylstannyl)pyridine (82 mg, 0.223 mmol) in 1,4-dioxane (1 mL) at 25° C. was degassed with nitrogen for 10 min and followed by addition of tetrakis(triphenylphosphine)palladium (1.430 mg, 1.237 mmol). The reaction mixture was stirred at 90° C. for 16 h before it was diluted with water (15 mL) and extracted with EtOAc (2×30 mL). The combined organic layer was dried over Na₂SO₄, filtered, and concentrated. The residue was purified by Prep-HPLC (Conditions: Luna OMEGA C-18 (250×21.2×5 μm), Mobile phase A: 0.1% formic acid in water (100%), Mobile phase B: 100% MeCN, Gradient: Time (min)/% B: 0/50, 10/80, Flow: 15 mL/min, Dilution: MeCN+H₂O). Pure fractions were combined and concentrated under reduced pressure to afford tert-butyl (R)-3-(2-fluoro-4-(1-methyl-1H-1,2,3-triazol-4-yl)-N-(6-(pyridin-2-yl)isoquinolin-1-yl)benzamido)piperidine-1-carboxylate (20 mg, 19.80%) as an off-white solid.

### Step 2:

The title compound hydrochloride salt was obtained as an off-white solid by deprotection Method A used in Example 97. LC-MS: [M+H⁺]=508.35. ¹H NMR (CD₃OD, 400 MHZ): δ 8.89 (d, J=5.6 Hz, 1H), 8.68-8.57 (m, 2H), 8.51-8.41 (m, 3H), 8.22-8.15 (m, 2H), 8.08-8.04 (m, 1H), 7.96-7.95 (m, 1H), 7.27-7.19 (m, 2H), 7.13-7.09 (m, 1H), 5.36-5.33 (m, 1H), 4.04 (s, 3H), 3.98-3.95 (m, 1H), 3.76-3.70 (m, 1H), 3.37-3.34 (m, 1H), 2.91-2.89 (m, 1H), 2.04-1.89 (m, 3H), 1.33-1.28 (m, 1H).

### Synthesis of (R)-2-fluoro-4-(1-methyl-1H-1,2,3-triazol-4-yl)-N-(6-(4-(methylsulfonyl)phenyl)isoquinolin-1-yl)-N-(piperidin-3-yl)benzamide (Example 582)

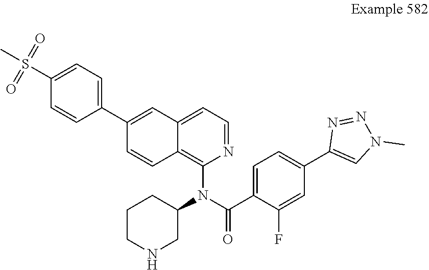

Example 582 was prepared according to the procedure described for Example 370 by substituting 3-pyridylboronic acid with (4-(methylsulfonyl) phenyl) boronic acid, provided the title compound hydrochloride salt as an off-white solid. LC-MS: [M+H⁺]=585.4. ¹H NMR (CD₃OD, 400 MHZ): δ 8.49 (d, J=5.6 Hz, 1H), 8.28-8.20 (m, 2H), 8.15-7.98 (m, 6H), 7.86-7.85 (m, 1H), 7.29-7.20 (m, 2H), 7.14-7.10 (m, 1H), 5.31-5.28 (m, 1H), 4.03 (s, 3H), 3.97-3.94 (m, 1H), 3.72-3.66 (m, 1H), 3.85-3.70 (m, 1H), 3.19 (s, 3H), 2.89-2.84 (m, 1H), 2.04-1.90 (m, 3H), 1.35-1.28 (m, 1H).

### Synthesis of (R)-4-(3H-[1,2,3]triazolo[4,5-b]pyridin-3-yl)-2-fluoro-N-(6-fluoro-8-methylisoquinolin-1-yl)-N-(piperidin-3-yl)benzamide (Example 583)

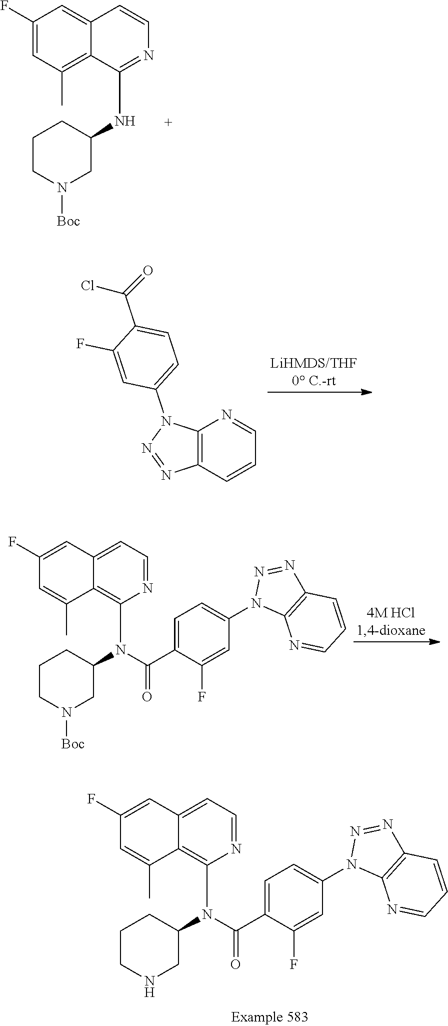 [SMILES: Cc1cc(F)cc2ccnc(N[C@@H]3CCCN(C(=O)OC(C)(C)C)C3)c12 ; O=C(Cl)c1ccc(-n2nnc3cccnc32)cc1F ; Cc1cc(F)cc2ccnc(N(C(=O)c3ccc(-n4nnc5cccnc54)cc3F)[C@@H]3CCCN(C(=O)OC(C)(C)C)C3)c12 ; Cc1cc(F)cc2ccnc(N(C(=O)c3ccc(-n4nnc5cccnc54)cc3F)[C@@H]3CCCNC3)c12]

### Step 1:

To a stirred solution of tert-butyl (R)-3-((6-fluoro-8-methylisoquinolin-1-yl)amino)piperidine-1-carboxylate (200 mg, 0.556 mmol) in THF (4 mL) at 0° C. was added LiHMDS in THF (1.19 mL, 1.669 mmol) and stirred for 15 min and followed by addition of 4-(3H-[1,2,3]triazolo[4,5-b]pyridin-3-yl)-2-fluoro benzoyl chloride (462 mg, 1.669 mmol) in THF (4 mL). The reaction mixture was stirred at room temperature for 30 min before it was quenched with saturated aq. NH₄Cl solution (10 mL) and extracted with EtOAc (60 mL). The organic layer was washed with brine, dried over Na₂SO₄, and concentrated. The residue was purified by Combi-flashy using 40-50% EtOAc in hexane first and then further purified by prep HPLC (Conditions: Luna OMEGA C-18 (250×21.2×5 μm), Mobile phase A: 0.1% formic acid in water (100%), Mobile phase B: 100% MeCN, Gradient: T/% B: 0/55, 10/80, Flow: 15 mL/min, Dilution: MeCN+H₂O). Pure fractions were dried by lyophilization to afford tert-butyl (R)-3-(4-(3H-[1,2,3]triazolo[4,5-b]pyridin-3-yl)-2-fluoro-N-(6-fluoro-8-methylisoquinolin-1-yl)benzamido) piperidine-1-carboxylate (60 mg, 65.4%) as an off-white solid. LC-MS: [M+H⁺]=600.5.

### Step 2:

The title compound hydrochloride salt was obtained as an off-white solid by deprotection Method A used in Example 97. LC-MS: [M+H⁺]=500.3. ¹H NMR (CD₃OD, 400 MHZ): δ 8.77-8.75 (m, 1H), 8.50-8.45 (m, 2H), 8.08-7.98 (m, 1H), 7.89-7.86 (m, 1H), 7.73-7.72 (m, 1H), 7.55-7.54 (m, 1H), 7.35-7.33 (m, 2H), 7.13-7.08 (m, 1H), 5.12-5.06 (m, 1H), 4.04-4.01 (m, 1H), 3.87-3.81 (m, 1H), 3.43-3.33 (m, 1H), 3.07-3.01 (m, 1H), 2.88 (s, 3H), 3.74-3.72 (m, 1H), 1.98-1.93 (m, 2H), 1.62-1.58 (m, 1H).

### Synthesis of (R)-4-(4-(4-(3H-[1,2,3]triazolo[4,5-b]pyridin-3-yl)-2-fluoro-N-(piperidin-3-yl)benzamido)thieno[3,2-c]pyridin-2-yl)-2,6-difluorobenzoic acid (Example 584)

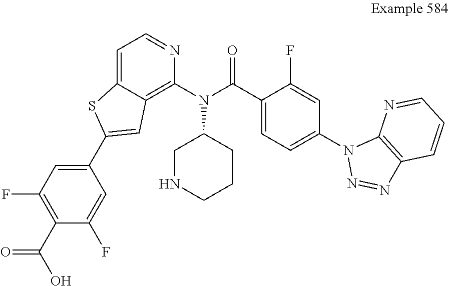 [SMILES: O=C(O)c1c(F)cc(-c2cc3c(N(C(=O)c4ccc(-n5nnc6cccnc65)cc4F)[C@@H]4CCCNC4)nccc3s2)cc1F]

Example 584 was prepared according to the procedure described for Example 161 by substituting 4-pyridylboronic acid with 2,6-difluoro-4-(4,4,5,5-tetramethyl-1,3,2-dioxaborolan-2-yl)benzoic acid, provided the title compound hydrochloride salt as a white solid. LC-MS: [M+H⁺]=629.3. ¹H NMR (400 MHZ, CD₃OD): δ 8.75 (br s, 1H), 8.50 (br d, J=7.9 Hz, 1H), 8.38 (br s, 1H), 8.06 (br d, J=10.4 Hz, 1H), 8.02-7.93 (m, 2H), 7.86 (br d, J=4.9 Hz, 1H), 7.54 (br d, J=8.3 Hz, 3H), 7.45-7.36 (m, 1H), 4.57 (br s, 1H), 3.66-3.54 (m, 1H), 3.27-3.16 (m, 1H), 3.03-2.91 (m, 1H), 2.54-2.33 (m, 2H), 2.10-1.96 (m, 1H), 1.78-1.64 (m, 2H), 1.35-1.20 (m, 1H).

### Synthesis of (R)-4-(3H-[1,2,3]triazolo[4,5-b]pyridin-3-yl)-2-fluoro-N-(2-(4-(2-hydroxypropan-2-yl)phenyl)thieno[3,2-c]pyridin-4-yl)-N-(piperidin-3-yl)benzamide (Example 585)

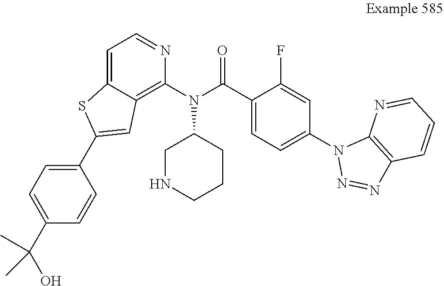 [SMILES: CC(C)(O)c1ccc(-c2cc3c(N(C(=O)c4ccc(-n5nnc6cccnc65)cc4F)[C@@H]4CCCNC4)nccc3s2)cc1]

Example 585 was prepared according to the procedure described for Example 161 by substituting 4-pyridylboronic acid with 2-[4-(4,4,5,5-tetramethyl-1,3,2-dioxaborolan-2-yl)phenyl]propan-2-ol, provided the title compound hydrochloride salt as a white solid. LC-MS: [M+H⁺]=608.4. ¹H NMR (400 MHZ, CD₃OD): δ 8.73 (br d, J=4.0 Hz, 1H), 8.49 (d, J=8.3 Hz, 1H), 8.36 (d, J=5.5 Hz, 1H), 8.10 (br d, J=10.8 Hz, 1H), 7.99 (br d, J=8.3 Hz, 1H), 7.87 (d, J=5.5 Hz, 1H), 7.79-7.70 (m, 3H), 7.63 (br d, J=8.3 Hz, 2H), 7.52 (dd, J=4.5, 8.3 Hz, 1H), 7.41 (br t, J=7.8 Hz, 1H), 5.33-5.20 (m, 1H), 3.97-3.83 (m, 1H), 3.67 (br t, J=11.9 Hz, 1H), 3.41-3.32 (m, 2H), 2.97-2.82 (m, 1H), 2.18-2.06 (m, 1H), 2.00-1.86 (m, 2H), 1.57 (s, 6H), 1.48-1.35 (m, 1H).

### Synthesis of (R)-4-(3H-[1,2,3]triazolo[4,5-b]pyridin-3-yl)-2-fluoro-N-(6-(3-hydroxy-3-methylbut-1-yn-1-yl)isoquinolin-1-yl)-N-(piperidin-3-yl)benzamide (Example 586)

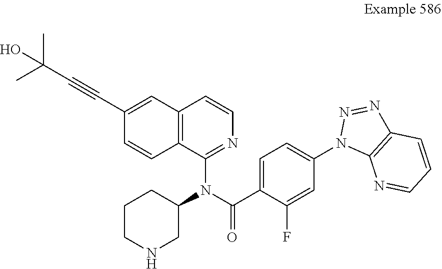 [SMILES: CC(C)(O)C#Cc1ccc2c(N(C(=O)c3ccc(-n4nnc5cccnc54)cc3F)[C@@H]3CCCNC3)nccc2c1]

Example 586 was prepared according to the procedure for Example 103 by substituting tert-butyl (R)-3-(4-(3H-[1,2,3]triazolo[4,5-b]pyridin-3-yl)-N-(7-bromoisoquinolin-1-yl)-2-fluorobenzamido) piperidine-1-carboxylate and prop-2-yn-ol with tert-butyl (R)-3-(4-(3H-[1,2,3]triazolo[4,5-b]pyridin-3-yl)-N-(6-bromoisoquinolin-1-yl)-2-fluorobenzamido)piperidine-1-carboxylate and tert-butyl (R)-3-(4-(3H-[1,2,3]triazolo[4,5-b]pyridin-3-yl)-N-(6-bromoisoquinolin-1-yl)-2-fluorobenzamido) piperidine-1-carboxylate, provided the title compound trifluoroacetic acid salt as an off-white solid. LC-MS: [M+H⁺]=550.3. ¹H NMR (CD₃OD, 400 MHZ): δ 8.76-8.74 (m, 1H), 8.50-8.42 (m, 2H), 8.15-8.13 (m, 1H), 8.06-8.03 (m, 1H), 7.95-7.87 (m, 2H), 7.72-7.65 (m, 2H), 7.54-7.50 (m, 1H), 7.37-7.28 (m, 1H), 5.29-5.23 (m, 1H), 3.97-3.90 (m, 1H), 3.73-3.67 (m, 1H), 3.36-3.38 (m, 1H), 2.90-2.83 (m, 1H), 1.99-1.90 (m, 3H), 1.50 (s, 6H), 1.31-1.25 (m, 1H).

### Synthesis of (R)-4-(3H-[1,2,3]triazolo[4,5-b]pyridin-3-yl)-2-fluoro-N-(6-(3-hydroxy-3-methylbut-1-yn-1-yl)-8-methylisoquinolin-1-yl)-N-(piperidin-3-yl)benzamide (Example 587)

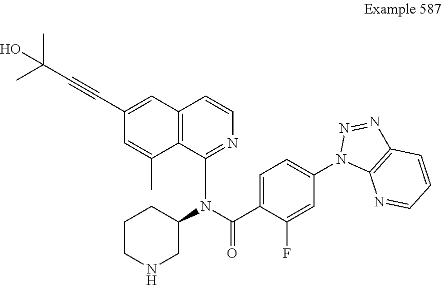 [SMILES: Cc1cc(C#CC(C)(C)O)cc2ccnc(N(C(=O)c3ccc(-n4nnc5cccnc54)cc3F)[C@@H]3CCCNC3)c12]

Example 597 was prepared according to the procedure described for Example 103 by substituting tert-butyl (R)-3-(4-(3H-[1,2,3]triazolo[4,5-b]pyridin-3-yl)-N-(7-bromoisoquinolin-1-yl)-2-fluorobenzamido)piperidine-1-carboxylate and prop-2-yn-ol with tert-butyl (R)-3-(4-(3H-[1,2,3]triazolo[4,5-b]pyridin-3-yl)-N-(6-bromoisoquinolin-1-yl)-2-fluorobenzamido)piperidine-1-carboxylate and tert-butyl (R)-3-(4-(3H-[1,2,3]triazolo[4,5-b]pyridin-3-yl)-N-(6-bromo-8-methylisoquinolin-1-yl)-2-fluorobenzamido) piperidine-1-carboxylate, provided the title compound hydrochloride salt as a white solid. LC-MS: [M+H⁺]=564.3. ¹H NMR (CD₃OD, 400 MHZ): δ 8.77-8.76 (m, 1H), 8.52-8.44 (m, 2H), 8.06-8.01 (m, 1H), 7.99-7.98 (m, 1H), 7.70-7.65 (m, 2H), 7.55-7.52 (m, 1H), 7.44-7.43 (m, 1H), 7.12-7.07 (m, 1H), 5.11-5.00 (m, 1H), 4.03-3.99 (m, 1H), 3.86-3.83 (m, 1H), 3.43-3.34 (m, 1H), 3.03-3.01 (m, 1H), 2.83 (s, 3H), 2.74-2.62 (m, 1H), 2.09-1.93 (m, 2H), 1.60-1.60 (m, 1H), 1.49 (s, 6H).

### Synthesis of (R)-3-(4-(1,3-dioxoisoindolin-2-yl)but-1-yn-1-yl)-N-(8-methylisoquinolin-1-yl)-N-(piperidin-3-yl)benzamide (Example 588)

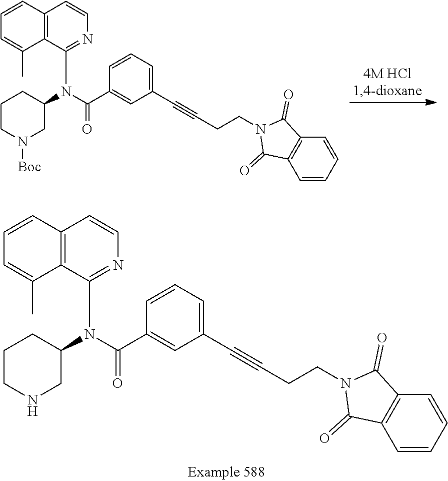 [SMILES: Cc1cccc2ccnc(N(C(=O)c3cccc(C#CCCN4C(=O)c5ccccc5C4=O)c3)[C@@H]3CCCN(C(=O)OC(C)(C)C)C3)c12 ; Cc1cccc2ccnc(N(C(=O)c3cccc(C#CCCN4C(=O)c5ccccc5C4=O)c3)[C@@H]3CCCNC3)c12]

To a solution of tert-butyl (R)-3-(3-(4-aminobut-1-yn-1-yl)-N-(8-methylisoquinolin-1-yl) benzamido) piperidine-1-carboxylate (40 mg, 0.078 mmol) in DCM (1 mL) was added 4M HCl in 1,4-dioxane (0.5 mL, 0.078 mmol) at 0° C. and stirred at room temperature for 2 h. The reaction mixture was concentrated under reduced pressure to afford (R)-3-(4-aminobut-1-yn-1-yl)-N-(8-methylisoquinolin-1-yl)-N-(piperidin-3-yl)benzamide hydrochloride salt (25 mg, 62.9%) as an off-white solid. LC-MS: [M+H⁺]=413.3. ¹H NMR (CD₃OD, 400 MHZ): δ 8.49 (d, J=5.6 Hz, 1H), 7.81 (d, J=5.6 Hz, 1H), 7.70-7.68 (m, 1H), 7.51-7.47 (m, 1H), 7.39-7.36 (m, 1H), 7.19 (s, 1H), 7.17-7.13 (m, 1H), 6.95-6.84 (m, 2H), 5.09-5.03 (m, 1H), 3.92-3.82 (m, 1H), 3.81-3.75 (m, 1H), 3.39-3.34 (m, 1H), 3.13-3.11 (m, 1H), 2.78-2.75 (m, 2H), 2.73 (s, 3H), 1.94-1.89 (m, 2H), 1.84-1.80 (m, 1H), 1.48-1.28 (m, 2H), 0.94-0.91 (m, 1H).

### Synthesis of methyl (R)-5-(3-((8-methylisoquinolin-1-yl)(piperidin-3-yl)carbamoyl)phenyl)pent-4-ynoate (Example 589)

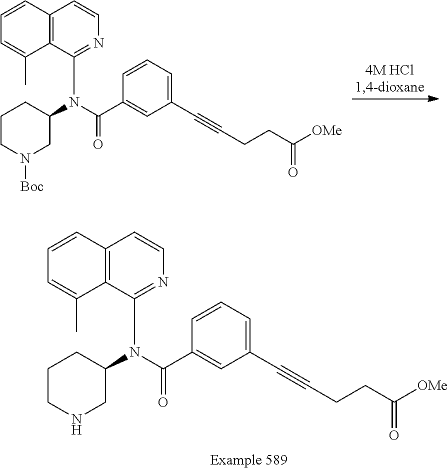 [SMILES: COC(=O)CCC#Cc1cccc(C(=O)N(c2nccc3cccc(C)c23)[C@@H]2CCCN(C(=O)OC(C)(C)C)C2)c1 ; COC(=O)CCC#Cc1cccc(C(=O)N(c2nccc3cccc(C)c23)[C@@H]2CCCNC2)c1]

To a solution of tert-butyl (R)-3-(3-(5-methoxy-5-oxopent-1-yn-1-yl)-N-(8-methylisoquinolin-1-yl) benzamido) piperidine-1-carboxylate (30 mg, 0.054 mmol) in DCM (1 mL) was added 4M HCl in 1,4-dioxane (2 mL) at 0° C. and stirred at room temperature for 3 h. The reaction mixture was concentrated under reduced pressure and dried by lyophilization for 2 days to afford methyl (R)-5-(3-((8-methylisoquinolin-1-yl)(piperidin-3-yl)carbamoyl)phenyl)pent-4-ynoate hydrochloride salt (25 mg, 93%) as an off-white solid. LC-MS: [M+H⁺]=456.2. ¹H NMR (CD₃OD, 400 MHZ): δ 8.48 (d, J=5.2 Hz, 1H), 7.80 (d, J=5.6 Hz, 1H), 7.72-7.66 (m, 2H), 7.51-7.47 (m, 1H), 7.38-7.36 (m, 1H), 7.04-7.02 (m, 1H), 6.97-6.85 (m, 2H), 5.10-5.02 (m, 1H), 3.94-3.91 (m, 1H), 3.81-3.76 (m, 1H), 3.71 (s, 3H), 3.47-3.36 (m, 1H), 3.01-2.97 (m, 1H), 2.71 (s, 3H), 2.61-2.56 (m, 4H), 1.94-1.81 (m, 2H), 1.52-1.28 (m, 2H).

### Synthesis of (R)-5-(3-((8-methylisoquinolin-1-yl)(piperidin-3-yl)carbamoyl)phenyl)pent-4-ynoic acid (Example 590)

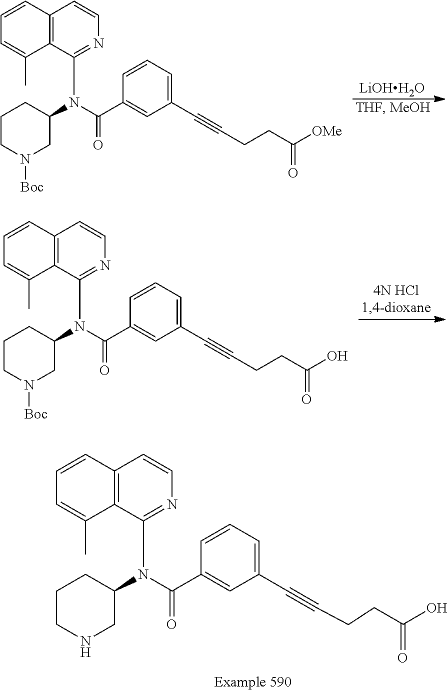

### Step 1:

To a stirred solution of tert-butyl (R)-3-(3-(5-methoxy-5-oxopent-1-yn-1-yl)-N-(8-methylisoquinolin-1-yl) benzamido) piperidine-1-carboxylate (67 mg, 0.121 mmol) in THF (2 mL), methanol (2 mL), water and (2 mL) was added lithium hydroxide mono-hydrate (25 mg, 0.603 mmol) at room temperature. The reaction was stirred for 4 h before it was concentrated under reduced pressure, diluted with water (30 mL), and extracted with EtOAc (2×50 mL). The combined organic layer was dried over Na₂SO₄ and concentrated. The residue was purified by Prep-HPLC (Conditions: Kinetex evo C-18 (250×21.2×5 μm), Mobile phase A: 0.1% formic acid in water (100%), Mobile phase B: 100% MeCN, Gradient: T/% B: 0/50, 10/80, Flow: 15 mL/min, Dilution: MeCN+H₂O). Pure fractions were concentrated under reduced pressure to afford (R)-5-(3-((1-(tert-butoxycarbonyl)piperidin-3-yl) (8-methylisoquinolin-1-yl) carbamoyl) phenyl) pent-4-ynoic acid (25 mg, 38.4%) as an off-white solid. LC-MS: [M+H⁺]=542.4.

### Step 2:

The title compound hydrochloride salt was obtained as an off-white solid by deprotection Method A used in Example 97. LC-MS: [M+H⁺]=442.25. ¹H NMR (CD₃OD, 400 MHZ): δ 8.49 (d, J=5.6 Hz, 1H), 7.81 (d, J=5.6 Hz, 1H), 7.72-7.68 (m, 1H), 7.50-7.46 (m, 1H), 7.38-7.36 (m, 1H), 7.04-7.02 (m, 1H), 6.94-6.86 (m, 3H), 5.10-5.03 (m, 1H), 3.94-3.91 (m, 1H), 3.82-3.79 (m, 1H), 3.39-3.36 (m, 1H), 3.01-2.97 (m, 1H), 2.70 (s, 3H), 2.61-2.58 (m, 2H), 2.54-2.51 (m, 2H), 1.98-1.82 (m, 2H), 1.53-1.28 (m, 2H).

### Synthesis of (R)-2-fluoro-N-(6-fluoroisoquinolin-1-yl)-N-(piperidin-3-yl)-4-(pyrimidin-2-ylamino)benzamide (Example 591)

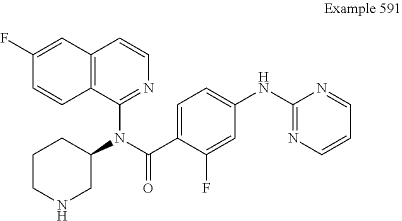 [SMILES: O=C(c1ccc(Nc2ncccn2)cc1F)N(c1nccc2cc(F)ccc12)[C@@H]1CCCNC1]

Example 591 was prepared according to the procedure described for Example 397 by substituting (R)-3-((6-bromoisoquinolin-1-yl)amino)piperidine-1-carboxylate with (R)-3-((6-fluoroisoquinolin-1-yl)amino)piperidine-1-carboxylate, provided the title compound hydrochloride salt as a white solid. LC-MS: [M+H⁺]=461.20. ¹H NMR (CD₃OD, 400 MHz): δ 8.57-8.56 (m, 2H), 8.44-8.43 (m, 1H), 8.22-8.20 (m, 1H), 7.82-7.76 (m, 1H), 7.59-7.40 (m, 3H), 7.05-6.97 (m, 3H), 5.23-4.96 (m, 1H), 3.92-3.85 (m, 1H), 3.72-3.69 (m, 1H), 3.34-3.30 (m, 1H), 2.89-2.86 (m, 1H), 1.97-1.88 (m, 3H), 1.30-1.28 (m, 1H).

### Synthesis of (R)-2-fluoro-4-(3-hydroxy-3-methylbut-1-yn-1-yl)-N-(8-methylisoquinolin-1-yl)-N-(piperidin-3-yl)benzamide (Example 592)

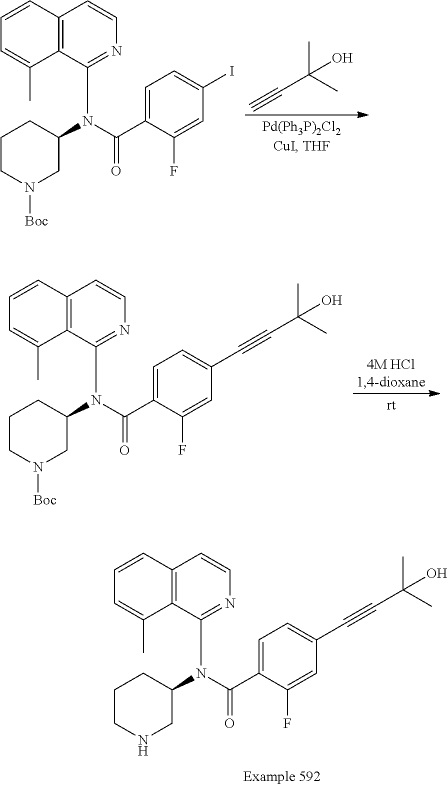

To a solution of tert-butyl (R)-3-(2-fluoro-4-iodo-N-(8-methylisoquinolin-1-yl) benzamido) piperidine-1-carboxylate (150 mg, 0.254 mmol) in THF (5 mL) was added piperidine (2 mL), 2-methylbut-3-yn-2-ol (23.55 mg, 0.280 mmol) and CuI (2.423 mg, 0.013 mmol) at room temperature. The reaction mixture was degassed with nitrogen for 10 min and followed by addition of bis(triphenylphosphine) palladium (II) chloride (8.93 mg, 0.013 mmol) was added to the reaction mixture and stirred at room temperature for 16 h. The reaction mixture was diluted with water (30 mL) and extracted with EtOAc (2×30 mL). The combined organic layer was dried over Na₂SO₄, and concentrated. The residue was purified by Combi-flash elutin with 50-60% EtOAc in hexane. The residue was further purified by Prep-HPLC (Conditions: Luna OMEGA C-18 (250×21.2×5 μm, Mobile phase A: 0.1% formic acid in water (100%), Mobile phase B: 100% MeCN, Gradient: Time (min)/% B: 0/55, 10/85, Flow: 15 mL/min, Dilution: MeCN+THF). Pure fractions were combined and concentrated under reduced pressure to afford tert-butyl (R)-3-(4-(3H-[1,2,3] triazolo[4,5-b]pyridin-3-yl)-2-fluoro-N-(6-(3-hydroxy-3-methylbut-1-yn-1-yl) isoquinolin-1-yl) benzamido) piperidine-1-carboxylate (45 mg, 32.1%) as a pale brown solid. LC-MS: [M+H⁺]=546.5.

### Step 2:

The title compound hydrochloride salt was obtained as an off-white solid by deprotection Method A used in Example 97. LC-MS: [M+H⁺]=446.2. ¹H NMR (CD₃OD, 400 MHZ): δ 8.40-8.37 (m, 1H), 7.73-7.64 (m, 2H), 7.54-7.45 (m, 2H), 6.78-6.70 (m, 2H), 6.68-6.64 (m, 1H), 5.05-4.96 (m, 1H), 3.98-3.95 (m, 1H), 3.81-3.75 (m, 1H), 3.40-3.37 (m, 1H), 3.02-2.97 (m, 1H), 2.69 (s, 3H), 1.95-1.82 (m, 3H), 1.61-1.57 (m, 1H), 1.44 (s, 6H).

### Synthesis of 2-fluoro-4-(5-methyl-1,3,4-thiadiazol-2-yl)-N-[(3R)-3-piperidyl]-N-[2-(4-pyridyl)thieno[3,2-c]pyridin-4-yl]benzamide (Example 593)

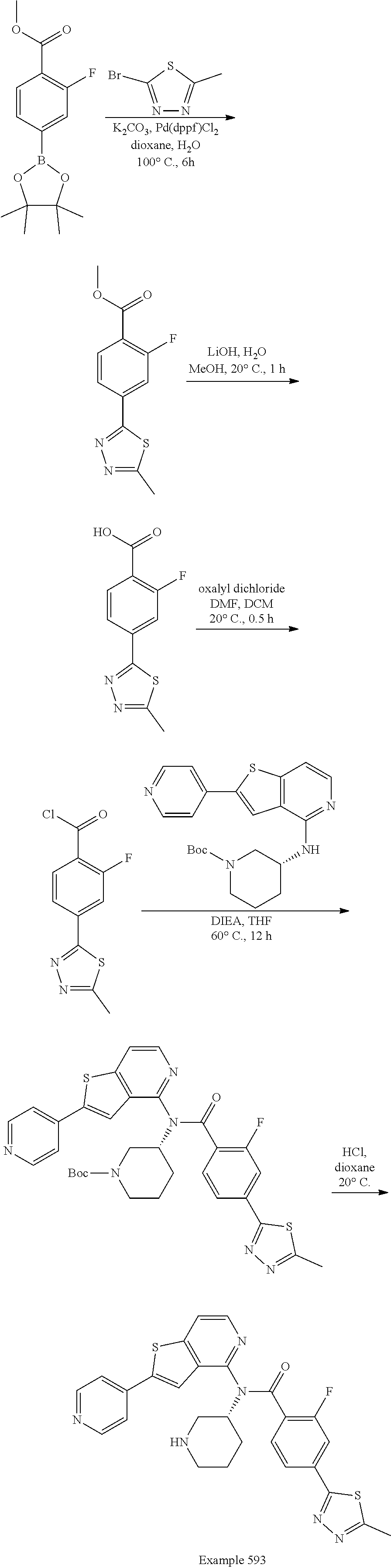

A mixture of 2-bromo-5-methyl-1,3,4-thiadiazole (159.80 mg, 892.54 μmol), methyl 2-fluoro-4-(4,4,5,5-tetramethyl-1,3,2-dioxaborolan-2-yl)benzoate (500 mg, 1.79 mmol), K₂CO₃ (493.42 mg, 3.57 mmol), Pd(dppf)Cl₂ (130.62 mg, 178.51 μmol) in dioxane (4.5 mL) and H₂O (0.5 mL) was degassed and purged with N₂, and then the mixture was stirred at 100° C. for 6 h under N₂ atmosphere. The reaction mixture was quenched with water (5 mL) at 0° C., and then extracted with ethyl acetate (3×5 mL). The combined organic layers were washed with brine (5 mL), dried over Na₂SO₄, filtered and concentrated. The residue was purified by column chromatography (SiO₂, Petroleum ether/Ethyl acetate=100/0 to 92/8) to give methyl 2-fluoro-4-(5-methyl-1,3,4-thiadiazol-2-yl)benzoate (190 mg, 37%) was obtained as a white solid.

### Step 2:

To a solution of methyl 2-fluoro-4-(5-methyl-1,3,4-thiadiazol-2-yl)benzoate (190 mg, 677.86 μmol) in H₂O (0.4 mL) and MeOH (2 mL) was added LiOH·H₂O (81.17 mg, 3.39 mmol). The mixture was stirred at 20° C. for 1 h before it was quenched with water (5 mL) at 0° C., and then the mixture was adjusted to pH5 with 1N aq. HCl. The mixture was extracted with ethyl acetate (3×5 mL). The combined organic layer was washed with brine (5 mL), dried over Na₂SO₄, filtered and concentrated under reduced pressure to give 2-fluoro-4-(5-methyl-1,3,4-thiadiazol-2-yl)benzoic acid (130 mg, 72%) as white solid.

### Step 3:

To a solution of 2-fluoro-4-(5-methyl-1,3,4-thiadiazol-2-yl)benzoic acid (130 mg, 491.11 μmol) in DCM (2 mL) was added DMF (358.97 μg, 4.91 μmol) and oxalyl dichloride (623.34 mg, 4.91 mmol). The mixture was stirred at 20° C. for 0.5 h. The reaction mixture was concentrated under reduced pressure to give 2-fluoro-4-(5-methyl-1,3,4-thiadiazol-2-yl)benzoyl chloride (130 mg, 92%) as brown solid.

### Step 4:

To a solution of tert-butyl (3R)-3-[[2-(4-pyridyl)thieno[3,2-c]pyridin-4-yl]amino]piperidine-1-carboxylate (74.85 mg, 182.33 μmol) in THF (2 mL) was added DIEA (117.82 mg, 911.63 μmol) and 2-fluoro-4-(5-methyl-1,3,4-thiadiazol-2-yl)benzoyl chloride (130 mg, 455.81 μmol). The mixture was stirred at 60° C. for 2 h. before it was filtered, and the filtrate was concentrated. The residue was purified by Prep-HPLC (column: Phenomenex luna C18 100*40 mm*5 μm; mobile phase: [H₂O (0.04% HCl)-MeCN]; gradient: 25%-55% B over 8.0 min) and lyophilized to afford tert-butyl (3R)-3-[[2-fluoro-4-(5-methyl-1,3,4-thiadiazol-2-yl)benzoyl]-[2-(4-pyridyl)thieno[3,2-c]pyridin-4-yl]amino]piperidine-1-carboxylate (30 mg, 9%) as a yellow solid.

### Step 5:

The title compound hydrochloride salt was obtained as an off-white solid by deprotection Method A used in Example 97. LC-MS: [M+H⁺]=531.3. ¹H NMR (400 MHZ, DMSO-d₆): δ 9.20-8.92 (m, 2H), 8.85 (br d, J=5.3 Hz, 2H), 8.63-8.53 (m, 1H), 8.43 (br d, J=5.5 Hz, 1H), 8.20-8.09 (m, 3H), 7.53 (br d, J=10.1 Hz, 1H), 7.47-7.33 (m, 2H), 5.20-4.92 (m, 1H), 3.73 (br d, J=10.6 Hz, 1H), 3.20 (br dd, J=2.3, 10.8 Hz, 2H), 2.70 (s, 3H), 2.12-2.02 (m, 1H), 1.96-1.70 (m, 3H), 1.20-1.07 (m, 1H).

### Synthesis of R)-4-(3H-[1,2,3]triazolo[4,5-b]pyridin-3-yl)-N-(2-(3-((1-amino-2-methyl-1-oxopropan-2-yl)oxy)phenyl)thieno[3,2-c]pyridin-4-yl)-2-fluoro-N-(piperidin-3-yl)benzamide (Example 594)

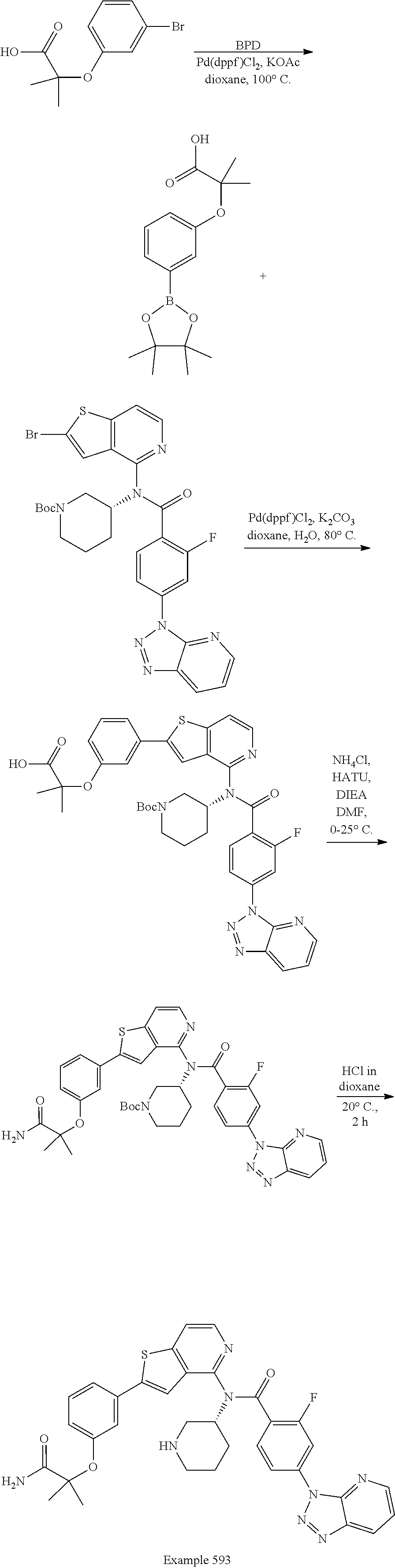

### Step 1:

To a solution of 2-(3-bromophenoxy)-2-methyl-propanoic acid (100 mg, 385.96 μmol) and KOAc (113.63 mg, 1.16 mmol) in dioxane (2 mL) at 20° C. under N₂ atmosphere was added Pd (dppf) Cl₂ (28.24 mg, 38.60 μmol) and BPD (196.02 mg, 771.91 μmol). The mixture was heated to 100° C. and stirred for 4 h. The reaction mixture was filtered and concentrated under reduced pressure to give 2-methyl-2-[3-(4, 4, 5, 5-tetramethyl-1, 3, 2-dioxaborolan-2-yl) phenoxy] propanoic acid (120 mg, 89.37%) as a dark oil. The crude product was used for the next step directly without purification.

### Step 2:

To a solution of tert-butyl (3R)-3-[(2-bromothieno[3, 2-c]pyridin-4-yl)-[2-fluoro-4-(triazolo [4, 5-b] pyridin-3-yl) benzoyl] amino] piperidine-1-carboxylate (200 mg, 306.50 μmol) and 2-methyl-2-[3-(4, 4, 5, 5-tetramethyl-1, 3, 2-dioxaborolan-2-yl) phenoxy] propanoic acid (112.60 mg, 367.80 μmol) in water (0.1 mL) and dioxane (2 mL) was added K₂CO₃ (127.08 mg, 919.49 μmol) and Pd(dppf)Cl₂ (22.43 mg, 30.65 μmol) at 20° C. The mixture was heated to 80° C. and stirred for 4 h under N₂ atmosphere. The reaction mixture was filtered and the filtrate was concentrated. The residue was purified by prep-TLC (SiO₂, ethyl acetate:methanol=10:1) to give 2-[3-[4-[[(3R)-1-tert-butoxycarbonyl-3-piperidyl]-[2-fluoro-4-(triazolo [4, 5-b] pyridin-3-yl) benzoyl] amino]thieno[3, 2-c] pyridin-2-yl] phenoxy]-2-methyl-propanoic acid (200 mg, 86%) as a white solid.

### Step 3:

To a solution of 2-[3-[4-[[(3R)-1-tert-butoxycarbonyl-3-piperidyl]-[2-fluoro-4-(triazolo [4, 5-b] pyridin-3-yl) benzoyl] amino] thieno [3, 2-c]pyridin-2-yl] phenoxy]-2-methyl-propanoic acid (180 mg, 239.42 μmol) and DIEA (92.83 mg, 718.25 μmol, 125.10 μL) in DMF (2 mL) at 0° C. was added NH₄Cl (38.42 mg, 718.25 μmol) and HATU (109.24 mg, 287.30 μmol). The mixture was stirred at 25° C. for 2 h. The reaction mixture was concentrated. The residue was purified by prep-TLC (SiO₂, Petroleum ether:Ethyl acetate=0:1) to give tert-butyl (3R)-3-[[2-[3-(2-amino-1,1-dimethyl-2-oxo-ethoxy) phenyl] thieno [3, 2-c] pyridin-4-yl]-[2-fluoro-4-(triazolo [4, 5-b] pyridin-3-yl) benzoyl] amino] piperidine-1-carboxylate (150 mg, 83%) as a white solid.

### Step 4:

A solution of tert-butyl (3R)-3-[[2-[3-(2-amino-1, 1-dimethyl-2-oxo-ethoxy) phenyl] thieno [3, 2-c] pyridin-4-yl]-[2-fluoro-4-(triazolo [4, 5-b] pyridin-3-yl) benzoyl] amino] piperidine-1-carboxylate (140 mg, 186.46 μmol) in 4M HCl in dioxane (3.23 mL) was stirred at 20° C. for 2 h and then was concentrated. The residue was purified by Prep-HPLC (column: Waters Xbridge Prep OBD C18 150*40 mm*10 μm; mobile phase: [H₂O (10 mM NH₄HCO₃)-MeCN]; gradient: 25%-70% B over 6.0 min) to give N-[2-[3-(2-amino-1,1-dimethyl-2-oxo-ethoxy) phenyl]thieno[3, 2-c] pyridin-4-yl]-2-fluoro-N-[(3R)-3-piperidyl]-4-(triazolo [4, 5-b] pyridin-3-yl) benzamide (21.8 mg, 18%) as a white solid. LC-MS: [M+H⁺]=651.4. ¹H NMR (400 MHZ, CD₃OD): δ 8.72 (br d, J=3.8 Hz, 1H), 8.47 (br d, J=8.3 Hz, 1H), 8.31 (br d, J=5.3 Hz, 1H), 8.06 (br d, J=11.0 Hz, 1H), 7.96 (br d, J=8.1 Hz, 1H), 7.79 (br d, J=5.3 Hz, 1H), 7.70 (br d, J=8.1 Hz, 2H), 7.65 (s, 1H), 7.50 (br dd, J=4.5, 8.3 Hz, 1H), 7.40 (br d, J=7.4 Hz, 1H), 7.07 (br d, J=8.5 Hz, 2H), 4.70-4.54 (m, 1H), 3.61-3.37 (m, 1H), 3.24-3.07 (m, 1H), 3.06-2.87 (m, 1H), 2.39 (br d, J=10.5 Hz, 1H), 2.08-1.91 (m, 1H), 1.79-1.63 (m, 2H), 1.58 (s, 6H), 1.37-1.20 (m, 1H).

### Synthesis of (R)-4-(3H-[1,2,3]triazolo[4,5-b]pyridin-3-yl)-N-(2-(4-(1-amino-2-methyl-1-oxopropan-2-yl)phenyl)thieno[3,2-c]pyridin-4-yl)-2-fluoro-N-(piperidin-3-yl)benzamide (Example 595)

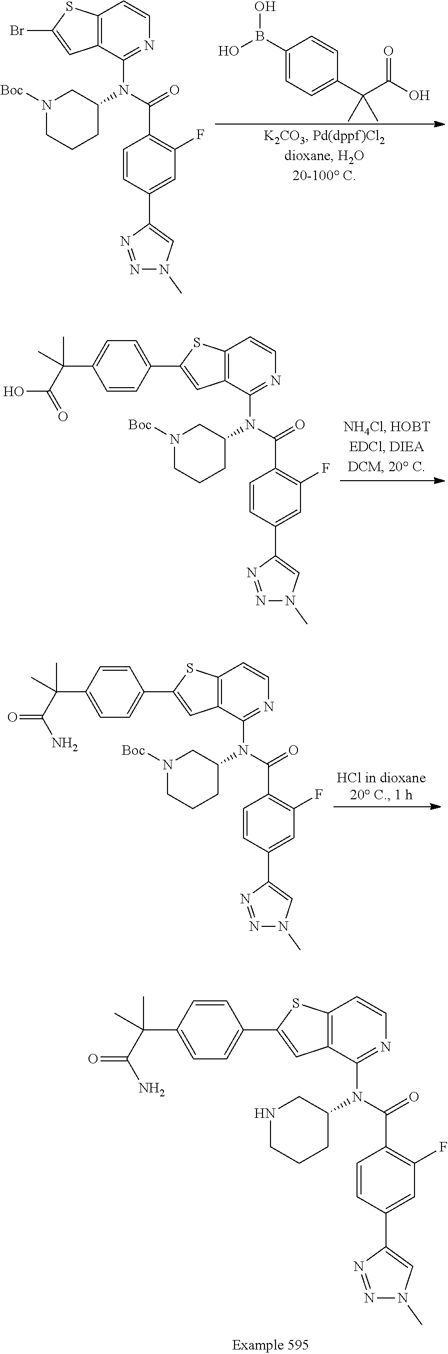 [SMILES: Cn1cc(-c2ccc(C(=O)N(c3nccc4sc(Br)cc34)[C@@H]3CCCN(C(=O)OC(C)(C)C)C3)c(F)c2)nn1 ; CC(C)(C(=O)O)c1ccc(B(O)O)cc1 ; Cn1cc(-c2ccc(C(=O)N(c3nccc4sc(-c5ccc(C(C)(C)C(=O)O)cc5)cc34)[C@@H]3CCCN(C(=O)OC(C)(C)C)C3)c(F)c2)nn1 ; Cn1cc(-c2ccc(C(=O)N(c3nccc4sc(-c5ccc(C(C)(C)C(N)=O)cc5)cc34)[C@@H]3CCCN(C(=O)OC(C)(C)C)C3)c(F)c2)nn1 ; Cn1cc(-c2ccc(C(=O)N(c3nccc4sc(-c5ccc(C(C)(C)C(N)=O)cc5)cc34)[C@@H]3CCCNC3)c(F)c2)nn1]

Example 595 was prepared according to the procedure for Example 594 by substituting 2-methyl-2-[3-(4, 4, 5, 5-tetramethyl-1, 3, 2-dioxaborolan-2-yl) phenoxy] propanoic acid with 2-(4-boronophenyl)-2-methyl-propanoic acid, provided the title compound hydrochloride salt as an off-white solid. LC-MS: [M+H⁺]=598.4. ¹H NMR (400 MHz, DMSO-d₆): δ 8.97-8.76 (m, 2H), 8.42 (s, 1H), 8.36-8.29 (m, 1H), 8.01-7.89 (m, 2H), 7.85-7.76 (m, 2H), 7.50-7.44 (m, 2H), 7.38 (br d, J=10.8 Hz, 1H), 7.31 (br d, J=8.1 Hz, 1H), 7.25-7.16 (m, 1H), 6.99 (br s, 2H), 5.17-4.90 (m, 1H), 3.99 (s, 3H), 3.78-3.65 (m, 1H), 3.19 (br d, J=9.5 Hz, 2H), 2.76-2.69 (m, 1H), 2.08-1.98 (m, 1H), 1.95-1.69 (m, 3H), 1.48 (s, 6H), 1.20-1.07 (m, 1H).

### Synthesis of (R)-4-(3H-[1,2,3]triazolo[4,5-b]pyridin-3-yl)-N-(2-(3-(1-amino-2-methyl-1-oxopropan-2-yl)phenyl)thieno[3,2-c]pyridin-4-yl)-2-fluoro-N-(piperidin-3-yl)benzamide (Example 596)

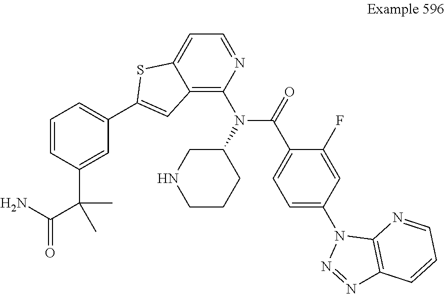 [SMILES: CC(C)(C(N)=O)c1cccc(-c2cc3c(N(C(=O)c4ccc(-n5nnc6cccnc65)cc4F)[C@@H]4CCCNC4)nccc3s2)c1]

Example 596 was prepared according to the procedure described for Example 161 by substituting 4-pyridylboronic acid with (3-(1-amino-2-methyl-1-oxopropan-2-yl)phenyl)boronic acid, provided the title compound hydrochloride salt as a white solid. LC-MS: [M+H⁺]=598.2. ¹H NMR (400 MHZ, DMSO-d₆): δ 9.10 (s, 2H), 8.45-8.29 (m, 2H), 8.03-7.74 (m, 4H), 7.50-7.18 (m, 4H), 6.99 (br s, 2H), 5.01 (s, 1H), 3.99 (s, 3H), 3.71 (br d, J=9.8 Hz, 1H), 3.25-3.11 (m, 1H), 2.84-2.63 (m, 1H), 2.07-1.71 (m, 3H), 1.48 (s, 6H), 1.25-1.03 (m, 1H).

### Synthesis of (R)—N-(2-(4-(1H-pyrazol-4-yl)phenyl)thieno[3,2-c]pyridin-4-yl)-4-(3H-[1,2,3]triazolo[4,5-b]pyridin-3-yl)-2-fluoro-N-(piperidin-3-yl)benzamide (Example 597)

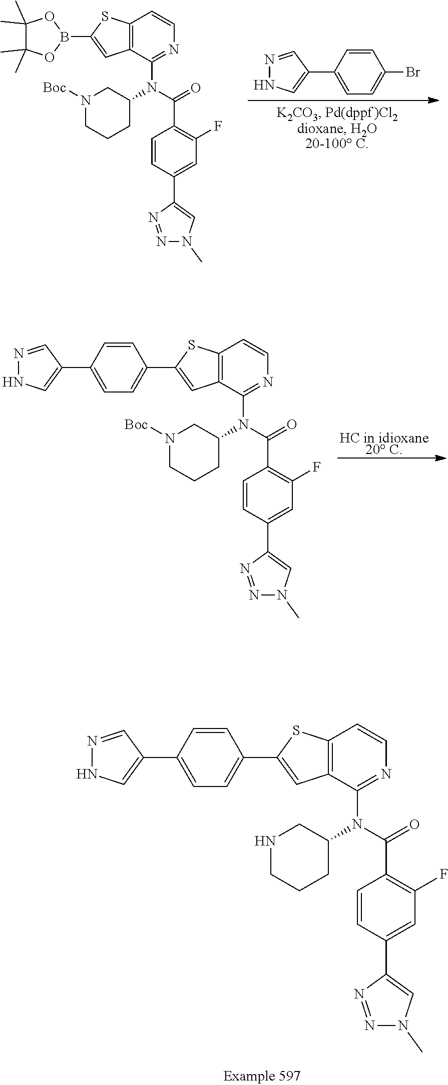

Example 597 was prepared according to the procedure described for Example 531 by substituting 4-bromo-N-methyl-pyrimidin-2-one with 4-(4-bromophenyl)-1H-pyrazole, provided the title compound as an off-white solid. LC-MS: [M+H⁺]=579.2. ¹H NMR (400 MHz, DMSO-d₆): δ 13.21-12.86 (m, 1H), 8.44-8.36 (m, 1H), 8.33-8.28 (m, 1H), 8.12-7.98 (m, 1H), 7.95-7.72 (m, 7H), 7.41-7.11 (m, 3H), 4.83-4.56 (m, 1H), 3.98 (s, 3H), 2.96-2.74 (m, 2H), 2.28-2.12 (m, 2H), 2.02-1.87 (m, 1H), 1.82-1.40 (m, 3H), 1.13-0.97 (m, 1H).

### Synthesis of (R)-4-(3H-[1,2,3]triazolo[4,5-b]pyridin-3-yl)-2-fluoro-N-(2-(4-fluorophenyl)thieno[3,2-c]pyridin-4-yl)-N-(piperidin-3-yl)benzamide (Example 598)

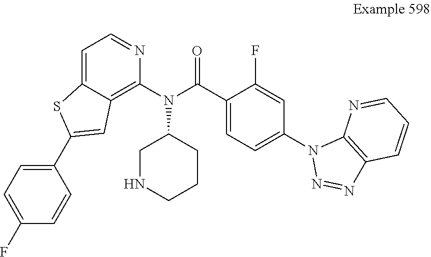 [SMILES: O=C(c1ccc(-n2nnc3cccnc32)cc1F)N(c1nccc2sc(-c3ccc(F)cc3)cc12)[C@@H]1CCCNC1]

Example 598 was prepared according to the procedure described for Example 161 by substituting 4-pyridylboronic acid with 2-(4-fluorophenyl)-4,4,5,5-tetramethyl-1,3,2-dioxaborolane, provided the title compound hydrochloride salt as a white solid. LC-MS: [M+H⁺]=531.3. ¹H NMR (400 MHZ, CD₃OD): δ 8.34 (d, J=5.5 Hz, 1H), 8.17 (s, 1H), 7.90-7.76 (m, 3H), 7.72-7.63 (m, 1H), 7.38-7.12 (m, 5H), 5.31-5.16 (m, 1H), 4.06 (s, 3H), 3.94-3.78 (m, 1H), 3.71-3.59 (m, 1H), 3.36 (br s, 1H), 2.87 (dt, J=3.8, 12.4 Hz, 1H), 2.49-2.17 (m, 1H), 2.07 (br d, J=11.9 Hz, 1H), 1.99-1.79 (m, 2H), 1.48-1.30 (m, 1H).

### Synthesis of (R)-4-(3H-[1,2,3]triazolo[4,5-b]pyridin-3-yl)-2-fluoro-N-(2-(4-(hydroxymethyl)phenyl)thieno[3,2-c]pyridin-4-yl)-N-(piperidin-3-yl)benzamide (Example 599)

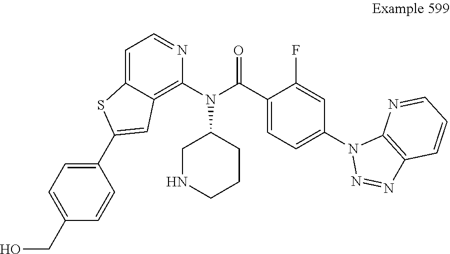 [SMILES: O=C(c1ccc(-n2nnc3cccnc32)cc1F)N(c1nccc2sc(-c3ccc(CO)cc3)cc12)[C@@H]1CCCNC1]

Example 500 was prepared according to the procedure described for Example 161 by substituting 4-pyridylboronic acid with [4-(hydroxymethyl)phenyl]boronic acid, provided the title compound hydrochloride salt as a white solid. LC-MS: [M+H⁺]=580.3. ¹H NMR (400 MHz, CD₃OD): δ 8.73 (br d, J=3.9 Hz, 1H), 8.49 (br d, J=8.1 Hz, 1H), 8.37 (d, J=5.4 Hz, 1H), 8.10 (br d, J=11.4 Hz, 1H), 7.99 (br d, J=8.4 Hz, 1H), 7.87 (br d, J=5.4 Hz, 1H), 7.80-7.73 (m, 3H), 7.55-7.47 (m, 3H), 7.41 (br t, J=7.8 Hz, 1H), 5.32-5.21 (m, 1H), 4.68 (s, 2H), 3.93 (br dd, J=1.3, 12.6 Hz, 1H), 3.72-3.63 (m, 1H), 3.41-3.32 (m, 2H), 2.96-2.83 (m, 1H), 2.19-2.05 (m, 1H), 2.03-1.82 (m, 2H), 1.52-1.32 (m, 1H).

### Synthesis of 4-(3H-[1,2,3]triazolo[4,5-b]pyridin-3-yl)-2-fluoro-N-(2-(4-(1-hydroxyethyl)phenyl)thieno[3,2-c]pyridin-4-yl)-N—((R)-piperidin-3-yl)benzamide (Example 600)

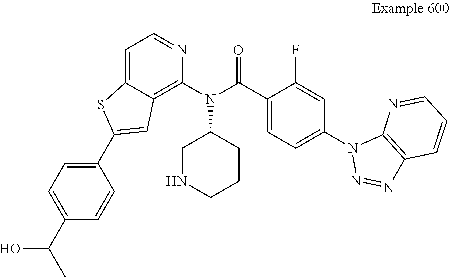 [SMILES: CC(O)c1ccc(-c2cc3c(N(C(=O)c4ccc(-n5nnc6cccnc65)cc4F)[C@@H]4CCCNC4)nccc3s2)cc1]

Example 600 was prepared according to the procedure described for Example 161 by substituting 4-pyridylboronic acid with [4-(1-hydroxyethyl)phenyl]boronic acid, provided the title compound hydrochloride salt as a white solid. LC-MS: [M+H⁺]=594.4. ¹H NMR (400 MHz, CD₃OD): δ 8.37 (d, J=5.5 Hz, 1H), 8.17 (s, 1H), 8.06-7.94 (m, 2H), 7.93-7.76 (m, 5H), 7.39-7.26 (m, 2H), 7.24-7.12 (m, 1H), 5.34-5.15 (m, 1H), 4.05 (s, 3H), 3.95-3.79 (m, 1H), 3.67 (br t, J=11.8 Hz, 1H), 3.35 (br d, J=13.3 Hz, 1H), 2.95-2.79 (m, 1H), 2.50-2.17 (m, 1H), 2.16-2.01 (m, 1H), 2.00-1.79 (m, 2H), 1.50-1.30 (m, 1H).

### Synthesis of 4-(3H-[1,2,3]triazolo[4,5-b]pyridin-3-yl)-2-fluoro-N-(2-(3-(1-hydroxyethyl)phenyl)thieno[3,2-c]pyridin-4-yl)-N—((R)-piperidin-3-yl)benzamide (Example 601)

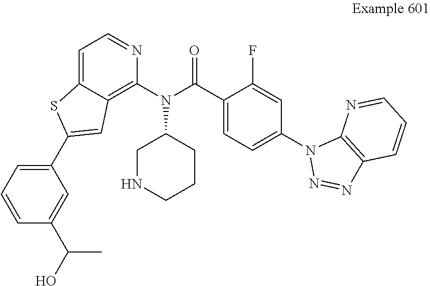 [SMILES: CC(O)c1cccc(-c2cc3c(N(C(=O)c4ccc(-n5nnc6cccnc65)cc4F)[C@@H]4CCCNC4)nccc3s2)c1]

Example 601 was prepared according to the procedure described for Example 161 by substituting 4-pyridylboronic acid with [3-(1-hydroxyethyl)phenyl]boronic acid, provided the title compound hydrochloride salt as a white solid. LC-MS: [M+H⁺]=594.4. ¹H NMR (400 MHz, CD₃OD): δ 8.73 (br d, J=3.9 Hz, 1H), 8.49 (br d, J=8.1 Hz, 1H), 8.37 (d, J=5.5 Hz, 1H), 8.11 (br d, J=10.9 Hz, 1H), 8.00 (br d, J=7.9 Hz, 1H), 7.88 (br d, J=5.4 Hz, 1H), 7.84-7.74 (m, 2H), 7.66 (br d, J=6.5 Hz, 1H), 7.56-7.38 (m, 4H), 5.35-5.21 (m, 1H), 4.93 (q, J=6.5 Hz, 1H), 4.00-3.82 (m, 1H), 3.68 (br t, J=11.9 Hz, 1H), 3.36 (br d, J=12.6 Hz, 1H), 2.89 (dt, J=4.0, 12.1 Hz, 1H), 2.54-2.20 (m, 1H), 2.11 (br d, J=10.4 Hz, 1H), 2.04-1.82 (m, 2H), 1.51 (d, J=6.5 Hz, 3H), 1.42 (br dd, J=3.9, 12.0 Hz, 1H).

### Synthesis of (R)-4-(3H-[1,2,3]triazolo[4,5-b]pyridin-3-yl)-2-fluoro-N-(2-(3-(2-hydroxypropan-2-yl)phenyl)thieno[3,2-c]pyridin-4-yl)-N-(piperidin-3-yl)benzamide (Example 602)

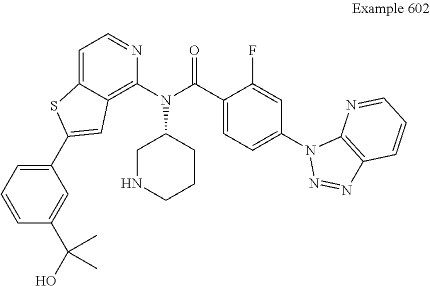 [SMILES: CC(C)(O)c1cccc(-c2cc3c(N(C(=O)c4ccc(-n5nnc6cccnc65)cc4F)[C@@H]4CCCNC4)nccc3s2)c1]

Example 602 was prepared according to the procedure described for Example 161 by substituting 4-pyridylboronic acid with [3-(1-hydroxy-1-methyl-ethyl)phenyl]boronic acid, provided the title compound hydrochloride salt as a white solid. LC-MS: [M+H⁺]=608.4. ¹H NMR (400 MHZ, CD₃OD): δ 8.74 (br d, J=3.9 Hz, 1H), 8.50 (br d, J=8.1 Hz, 1H), 8.38 (d, J=5.5 Hz, 1H), 8.13 (br d, J=10.9 Hz, 1H), 8.00 (br d, J=8.3 Hz, 1H), 7.93 (br s, 1H), 7.88 (d, J=5.5 Hz, 1H), 7.77 (s, 1H), 7.63 (br d, J=7.5 Hz, 1H), 7.59-7.39 (m, 4H), 5.28 (br d, J=1.9 Hz, 1H), 3.93 (br dd, J=1.2, 11.3 Hz, 1H), 3.74-3.64 (m, 1H), 3.37 (br s, 1H), 2.95-2.83 (m, 1H), 2.16-2.06 (m, 1H), 2.01-1.84 (m, 2H), 1.61 (s, 6H), 1.48-1.35 (m, 1H).

### Synthesis of N-[2-(4-acetylphenyl)thieno[3,2-c]pyridin-4-yl]-2-fluoro-N-[(3R)-3-piperidyl]-4-(triazolo[4,5-b]pyridin-3-yl) benzamide (Example 603)

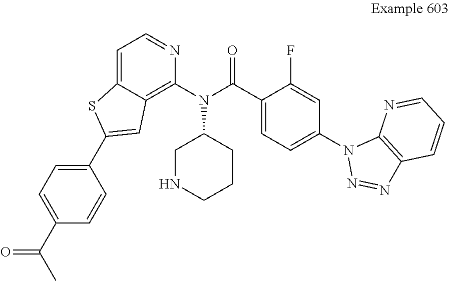 [SMILES: CC(=O)c1ccc(-c2cc3c(N(C(=O)c4ccc(-n5nnc6cccnc65)cc4F)[C@@H]4CCCNC4)nccc3s2)cc1]

Example 603 was prepared according to the procedure described for Example 161 by substituting 4-pyridylboronic acid with (4-acetylphenyl) boronic acid, provided the title compound hydrochloride salt as a white solid. LC-MS: [M+H⁺]=592.4. ¹H NMR (400 MHz, CD3OD): δ 8.72 (br s, 1H), 8.49 (br d, J=8.1 Hz, 1H), 8.43-8.34 (m, 1H), 8.16-8.06 (m, 2H), 8.04-7.85 (m, 4H), 7.83-7.74 (m, 1H), 7.65-7.36 (m, 2H), 5.36-5.22 (m, 1H), 3.99-3.83 (m, 1H), 3.76-3.62 (m, 1H), 3.41-3.33 (m, 1H), 2.98-2.83 (m, 1H), 2.65 (s, 2H), 2.53-2.20 (m, 1H), 2.19-2.06 (m, 1H), 2.05-1.81 (m, 2H), 1.55 (s, 1H), 1.49-1.33 (m, 1H).

### Synthesis of (R)-2-fluoro-N-(2-(4-(1,1,1,3,3,3-hexafluoro-2-hydroxypropan-2-yl)phenyl)thieno[3,2-c]pyridin-4-yl)-4-(1-methyl-1H-1,2,3-triazol-4-yl)-N-(piperidin-3-yl)benzamide (Example 604)

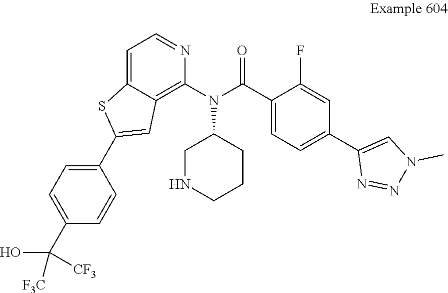 [SMILES: Cn1cc(-c2ccc(C(=O)N(c3nccc4sc(-c5ccc(C(O)(C(F)(F)F)C(F)(F)F)cc5)cc34)[C@@H]3CCCNC3)c(F)c2)nn1]

Example 604 was prepared according to the procedure described for Example 363 by substituting 4-pyridylboronic acid with 1,1,1,3,3,3-hexafluoro-2-(4-(4,4,5,5-tetramethyl-1,3,2-dioxaborolan-2-yl)phenyl) propan-2-ol, provided the title compound hydrochloride salt as a white solid. LC-MS: [M+H⁺]=679.3. ¹H NMR (400 MHZ, DMSO-d₆): δ 8.99-8.86 (m, 1H), 8.45-8.30 (m, 2H), 8.03 (br d, J=8.0 Hz, 3H), 7.96 (br d, J=5.1 Hz, 1H), 7.83 (br d, J=7.3 Hz, 2H), 7.43-7.17 (m, 3H), 4.85-4.51 (m, 1H), 4.06-3.92 (m, 3H), 2.93-2.76 (m, 2H), 2.24-2.10 (m, 2H), 2.01-1.90 (m, 1H), 1.84-1.62 (m, 1H), 1.59-1.40 (m, 2H), 0.82 (s, 1H). ¹⁹F NMR (376 MHz, DMSO-d₆): δ −73.82 (br s, 6F), −113.24-−114.34 (m, 1F).

### Synthesis of 2-fluoro-4-(1-methyl-1H-1,2,3-triazol-4-yl)-N—((R)-piperidin-3-yl)-N-(2-(4-(2,2,2-trifluoro-1-hydroxyethyl)phenyl)thieno[3,2-c]pyridin-4-yl)benzamide (Example 605)

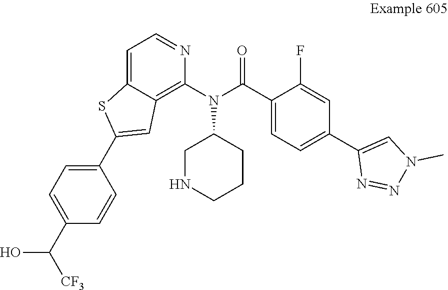 [SMILES: Cn1cc(-c2ccc(C(=O)N(c3nccc4sc(-c5ccc(C(O)C(F)(F)F)cc5)cc34)[C@@H]3CCCNC3)c(F)c2)nn1]

Example 605 was prepared according to the procedure described for Example 363 by substituting 4-pyridylboronic acid with 2,2,2-trifluoro-1-[4-(4,4,5,5-tetramethyl-1,3,2-dioxaborolan-2-yl)phenyl]ethanol, provided the title compound hydrochloride salt as a white solid. LC-MS: [M+H⁺]=611.3. ¹H NMR (400 MHZ, CD₃OD): δ 8.35 (d, J=5.4 Hz, 1H), 8.16 (s, 1H), 7.86 (br d, J=5.4 Hz, 1H), 7.84-7.73 (m, 3H), 7.64 (br d, J=8.0 Hz, 2H), 7.38-7.26 (m, 2H), 7.25-7.13 (m, 1H), 5.31-5.18 (m, 1H), 5.12 (q, J=7.0 Hz, 1H), 4.05 (s, 3H), 3.94-3.81 (m, 1H), 3.73-3.61 (m, 1H), 3.36 (br s, 1H), 2.87 (dt, J=3.6, 12.2 Hz, 1H), 2.50-2.17 (m, 1H), 2.16-2.01 (m, 1H), 2.00-1.80 (m, 2H), 1.47-1.31 (m, 1H).

### Synthesis of (R)-4-(3H-[1,2,3]triazolo[4,5-b]pyridin-3-yl)-N-(2-(4-carbamoyl-3,5-dimethylphenyl)thieno[3,2-c]pyridin-4-yl)-2-fluoro-N-(piperidin-3-yl)benzamide (Example 606)

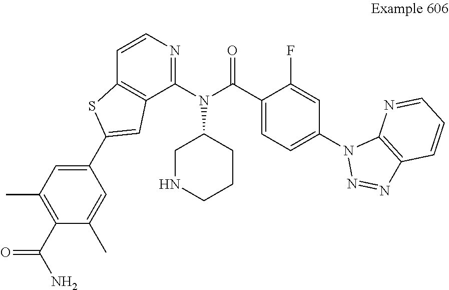 [SMILES: Cc1cc(-c2cc3c(N(C(=O)c4ccc(-n5nnc6cccnc65)cc4F)[C@@H]4CCCNC4)nccc3s2)cc(C)c1C(N)=O]

Example 606 was prepared according to the procedure described for Example 161 by substituting 4-pyridylboronic acid with 2, 6-dimethyl-4-(4, 4, 5, 5-tetramethyl-1, 3, 2-dioxaborolan-2-yl) benzoic acid, provided the title compound hydrochloride salt as a white solid. LC-MS: [M+H⁺]=621.3. ¹H NMR (400 MHZ, CD₃OD): δ 8.75 (br d, J=4.1 Hz, 1H), 8.50 (br d, J=8.1 Hz, 1H), 8.38 (d, J=5.5 Hz, 1H), 8.09 (br d, J=11.1 Hz, 1H), 7.99 (br d, J=8.1 Hz, 1H), 7.87 (d, J=5.4 Hz, 1H), 7.83-7.73 (m, 1H), 7.55-7.46 (m, 3H), 7.40 (br t, J=7.8 Hz, 1H), 5.37-5.20 (m, 1H), 3.93 (br d, J=12.1 Hz, 1H), 3.68 (br t, J=11.6 Hz, 1H), 3.34 (br s, 1H), 2.96-2.81 (m, 1H), 2.44 (s, 6H), 2.20-2.06 (m, 1H), 2.03-1.82 (m, 2H), 1.49-1.33 (m, 1H).

### Synthesis of (R)—N-(2-(4-carbamoyl-3-methylphenyl)thieno[3,2-c]pyridin-4-yl)-2-fluoro-4-(1-methyl-1H-1,2,3-triazol-4-yl)-N-(piperidin-3-yl)benzamide (Example 607)

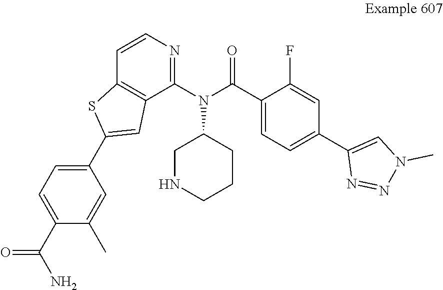 [SMILES: Cc1cc(-c2cc3c(N(C(=O)c4ccc(-c5cn(C)nn5)cc4F)[C@@H]4CCCNC4)nccc3s2)ccc1C(N)=O]

Example 607 was prepared according to the procedure described for Example 363 by substituting 4-pyridylboronic acid with 2-methyl-4-(4,4,5,5-tetramethyl-1,3,2-dioxaborolan-2-yl)benzoic acid, provided the title compound hydrochloride salt as a white solid. LC-MS: [M+H⁺]=570.4. ¹H NMR (400 MHZ, CD3OD): δ 8.36 (d, J=5.5 Hz, 1H), 8.17 (s, 1H), 7.87 (br d, J=5.4 Hz, 1H), 7.82-7.73 (m, 1H), 7.71-7.60 (m, 2H), 7.55 (d, J=8.0 Hz, 1H), 7.38-7.24 (m, 2H), 7.18 (br d, J=6.9 Hz, 1H), 5.31-5.18 (m, 1H), 4.12-4.03 (m, 3H), 3.93-3.81 (m, 1H), 3.72-3.61 (m, 1H), 3.42-3.35 (m, 1H), 2.95-2.82 (m, 1H), 2.54 (s, 3H), 2.47-2.21 (m, 1H), 2.13-2.02 (m, 1H), 1.99-1.81 (m, 2H), 1.46-1.31 (m, 1H).

### Synthesis of (R)—N-(2-(4-carbamoyl-3,5-dimethylphenyl)thieno[3,2-c]pyridin-4-yl)-2-fluoro-4-(1-methyl-1H-1,2,3-triazol-4-yl)-N-(piperidin-3-yl)benzamide (Example 608)

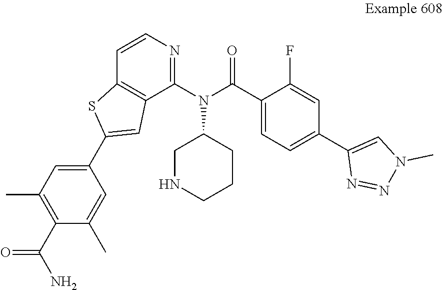

Example 608 was prepared according to the procedure described for Example 363 by substituting 4-pyridylboronic acid with 2,6-difluoro-4-(4,4,5,5-tetramethyl-1,3,2-dioxaborolan-2-yl)benzoic acid, provided the title compound hydrochloride salt as a white solid. LC-MS: [M+H⁺]=592.3. ¹H NMR (400 MHZ, DMSO-d₆): δ 9.13-8.73 (m, 2H), 8.45-8.34 (m, 2H), 8.32-8.23 (m, 1H), 8.20 (s, 1H), 8.04 (d, J=5.8 Hz, 1H), 7.96 (br s, 1H), 7.82-7.69 (m, 2H), 7.40-7.20 (m, 3H), 5.18-4.90 (m, 1H), 4.00 (s, 3H), 3.80-3.76 (m, 1H), 3.26-3.16 (m, 1H), 2.82-2.71 (m, 1H), 2.11-2.01 (m, 1H), 1.96-1.65 (m, 3H), 1.17-1.04 (m, 1H).

### Synthesis of (R)—N-(6-benzylisoquinolin-1-yl)-2-fluoro-4-(1-methyl-1H-1,2,3-triazol-4-yl)-N-(piperidin-3-yl)benzamide (Example 609)

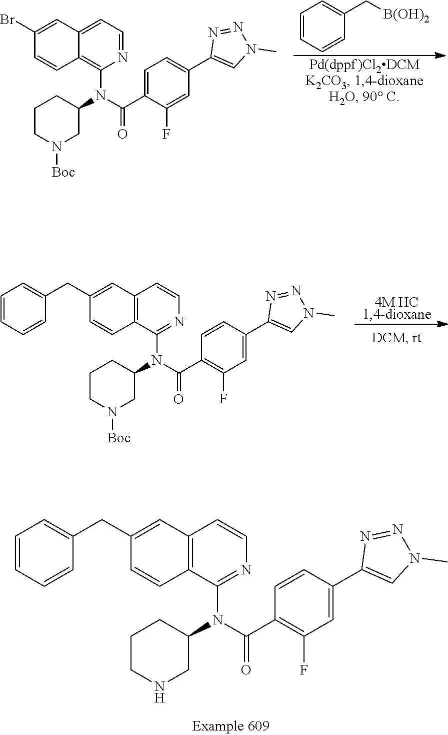

Example 609 was prepared according to the procedure described for Example 370 by substituting 3-pyridylboronic acid with (4-fluorophenyl) boronic acid with benzylboronic acid, provided the title compound hydrochloride salt as an off-white solid. LC-MS: [M+H⁺]=521.3. ¹H NMR (CD₃OD, 400 MHZ): δ 8.39 (d, J=5.6 Hz, 1H), 8.18 (s, 1H), 8.09-8.02 (m, 1H), 7.79-7.49 (m, 3H), 7.25-7.05 (m, 8H), 5.22-5.20 (m, 1H), 4.21 (s, 2H), 4.20 (s, 3H), 3.93-3.90 (m, 1H), 3.71-3.65 (m, 1H), 2.87-2.85 (m, 1H), 2.39-2.30 (m, 1H), 2.05-1.87 (m, 3H), 1.39-1.28 (m, 1H).

### Synthesis of (R)—N-(6-acetylisoquinolin-1-yl)-2-fluoro-4-(1-methyl-1H-1,2,3-triazol-4-yl)-N-(piperidin-3-yl)benzamide (Example 610)

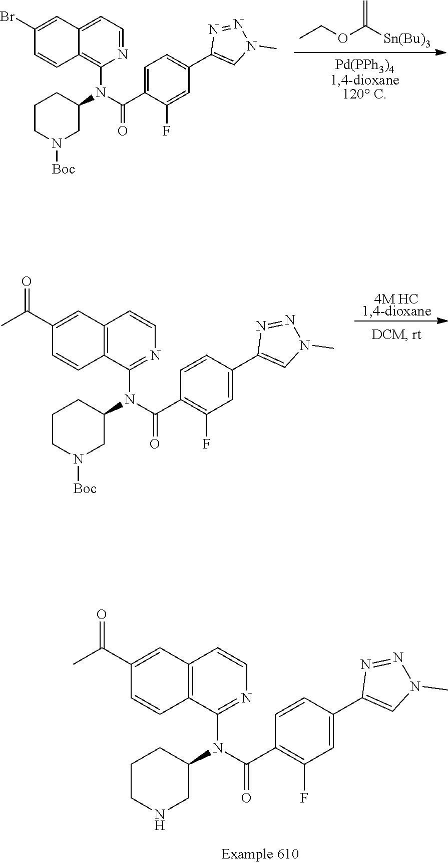

Example 2430 was prepared according to the procedure described for Example 581 by substituting 4-pyridylboronic acid with benzylboronic acid, provided the title compound formate salt as an off-white solid. LC-MS: [M+H⁺]=473.20. ¹H NMR (CD₃OD, 400 MHZ): δ 8.54-8.49 (m, 2H), 8.23-8.13 (m, 3H), 7.92-7.90 (m, 1H), 7.25-7.17 (m, 2H), 7.09-7.05 (m, 1H), 5.22-5.20 (m, 1H), 4.04 (s, 3H), 3.91-3.87 (m, 1H), 3.65-3.59 (m, 1H), 3.26-3.20 (m, 1H), 2.82-2.78 (m, 1H), 2.66 (s, 3H), 1.98-1.88 (m, 3H), 1.37-1.33 (m, 1H).

### Synthesis of (R)—N-(2-(4-carbamoyl-3,5-dimethylphenyl)thieno[3,2-c]pyridin-4-yl)-2-fluoro-4-(1-methyl-1H-1,2,3-triazol-4-yl)-N-(piperidin-3-yl)benzamide (Example 611)

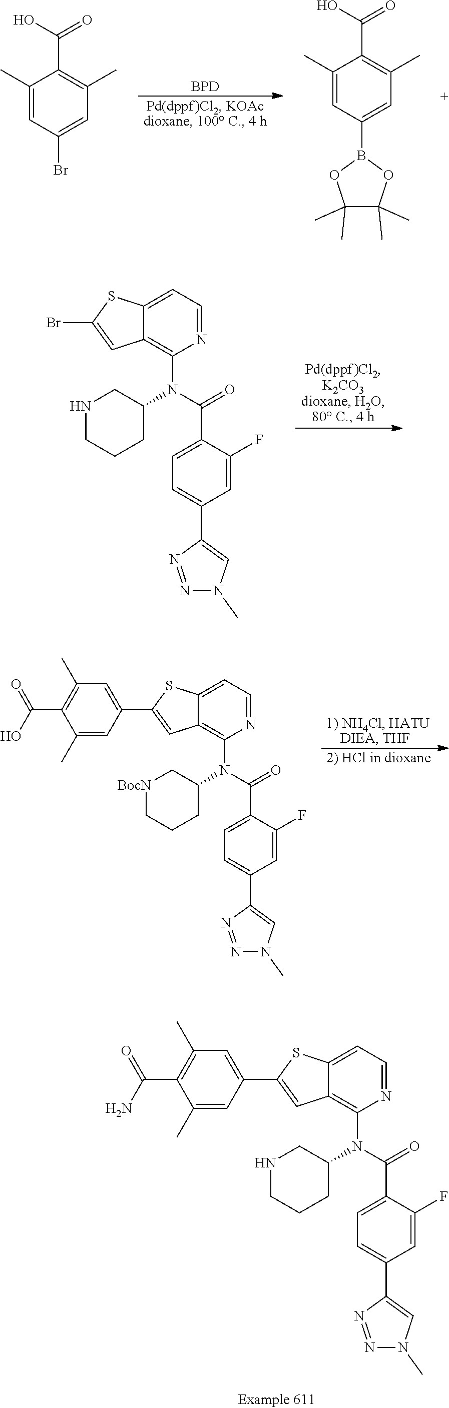

Example 2431 was prepared according to the procedure for Example 594 by substituting 2-(3-bromophenoxy)-2-methyl-propanoic acid and tert-butyl (3R)-3-[(2-bromothieno [3, 2-c] pyridin-4-yl)-[2-fluoro-4-(triazolo [4, 5-b] pyridin-3-yl) benzoyl] amino] piperidine-1-carboxylate with 4-bromo-2,6-dimethylbenzoic acid and (R)—N-(2-bromothieno[3,2-c]pyridin-4-yl)-2-fluoro-4-(1-methyl-1H-1,2,3-triazol-4-yl)-N-(piperidin-3-yl)benzamide, provided the title compound hydrochloride salt as a white solid. LC-MS: [M+H⁺]=584.4. ¹H NMR (400 MHZ, CD₃OD): δ 8.31 (br d, J=5.4 Hz, 1H), 8.16 (s, 1H), 7.81 (br d, J=4.6 Hz, 1H), 7.71 (s, 1H), 7.48 (s, 2H), 7.37-7.21 (m, 2H), 7.21-7.10 (m, 1H), 4.58 (br d, J=1.0 Hz, 1H), 4.06 (s, 3H), 3.63-3.44 (m, 1H), 3.25-3.08 (m, 1H), 2.97 (br d, J=9.5 Hz, 1H), 2.44 (s, 6H), 2.40 (br d, J=3.4 Hz, 1H), 2.11-1.91 (m, 1H), 1.86-1.61 (m, 2H), 1.42-1.16 (m, 1H).

### Synthesis of (R)—N-(2-(4-carbamoylphenyl)thieno[3,2-c]pyridin-4-yl)-4-(1,5-dimethyl-1H-pyrazol-4-yl)-2-fluoro-N-(piperidin-3-yl)benzamide (Example 612)

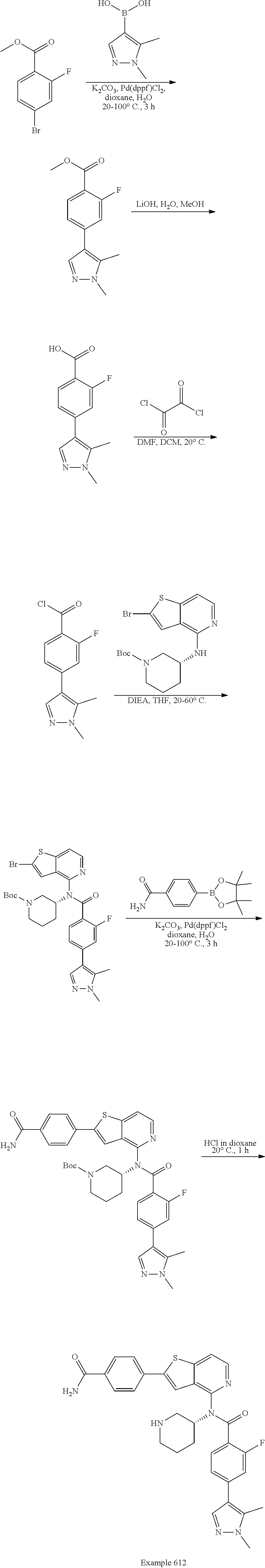

### Step 1:

To a solution of methyl 4-bromo-2-fluoro-benzoate (500 mg, 2.15 mmol) and (1,5-dimethylpyrazol-4-yl)boronic acid (600.55 mg, 4.29 mmol) in dioxane (9 mL) and H₂O (1 mL) was added K₂CO₃ (889.63 mg, 6.44 mmol) and Pd(dppf)Cl₂ (157.00 mg, 214.56 μmol) at 20° C. under N₂ atmosphere. The mixture was stirred at 100° C. for 3 before it was poured into water (20 mL) and extracted with ethyl acetate (3×20 mL). The combined organic phase was washed with brine (50 mL), dried with anhydrous Na₂SO₄, concentrated in vacuum to give residue. The residue was purified by column chromatography (SiO₂, Petroleum ether/Ethyl acetate=95/5 to 90/10) to give methyl 4-(1,5-dimethylpyrazol-4-yl)-2-fluoro-benzoate (0.8 g, 71%) as a light brown solid. LC-MS: [M+H⁺]=249.1.

### Step 2:

To a solution of methyl 4-(1,5-dimethylpyrazol-4-yl)-2-fluoro-benzoate (700 mg, 2.68 mmol) in H₂O (4 mL) and MeOH (8 mL) was added LiOH·H₂O (562.00 mg, 13.39 mmol) at 20° C. under N₂. The mixture was stirred at 20° C. for 1 before it was adjusted to pH 6 with 1N aq. HCl. The mixture was extracted with ethyl acetate (3×50 mL). The combined organic phase was washed with brine (100 mL), dried by anhydrous Na₂SO₄, concentrated to give 4-(1,5-dimethylpyrazol-4-yl)-2-fluoro-benzoic acid (0.55 g, 83%) as a brown solid. LC-MS: [M+H⁺]=235.1.

### Step 3:

To a solution of 4-(1,5-dimethylpyrazol-4-yl)-2-fluoro-benzoic acid (250 mg, 1.01 mmol) in DCM (6 mL) was added oxalyl dichloride (1.29 g, 10.14 mmol) and DMF (3.71 mg, 50.70 μmol, 3.90 μL) dropwise at 20° C. under N₂ Atmosphere. The mixture was stirred at 20° C. for 0.5. The reaction mixture was concentrated to give 4-(1,5-dimethylpyrazol-4-yl)-2-fluoro-benzoyl chloride (250 mg, 92%) as a light yellow solid.

### Step 4:

To a solution of 4-(1,5-dimethylpyrazol-4-yl)-2-fluoro-benzoyl chloride (250 mg, 939.96 μmol) and tert-butyl (3R)-3-[(2-bromothieno[3,2-c]pyridin-4-yl)amino]piperidine-1-carboxylate (155.03 mg, 375.98 μmol) in THF (10 mL) was added DIEA (607.41 mg, 4.70 mmol) at 20° C. under N₂Atmosphere. The mixture was stirred at 60° C. for 4 mixture before it was concentrated. The residue was purified by column chromatography (SiO₂, Petroleum ether/Ethyl acetate=50/50 to 40/60) to give tert-butyl (3R)-3-[(2-bromothieno[3,2-c]pyridin-4-yl)-[4-(1,5-dimethylpyrazol-4-yl)-2-fluoro-benzoyl]amino]piperidine-1-carboxylate (0.3 g, 40%) as a pale-yellow oil.

### Steps 5-6:

Example 2432 was prepared according to the procedure for Example 161 by substituting 4-pyridylboronic acid with 4-(4,4,5,5-tetramethyl-1,3,2-dioxaborolan-2-yl)benzamide, provided the title compound hydrochloride salt as an off-white solid. LC-MS: [M+H⁺]=569.4. ¹H NMR (400 MHZ, DMSO-d₆): δ 9.11-8.77 (m, 2H), 8.36 (br d, J=5.5 Hz, 1H), 8.10 (br s, 2H), 8.06-7.79 (m, 6H), 7.54-7.45 (m, 2H), 7.20-7.08 (m, 1H), 7.03-6.83 (m, 2H), 5.21-4.84 (m, 1H), 3.78-3.71 (m, 1H), 3.69-3.62 (m, 3H), 3.25-3.13 (m, 1H), 2.78-2.66 (m, 1H), 2.15 (s, 3H), 2.09-2.00 (m, 1H), 1.78 (br d, J=1.8 Hz, 3H), 1.20-1.08 (m, 1H).

### Synthesis of N-(2-(4-(1-amino-2,2,2-trifluoroethyl)phenyl)thieno[3,2-c]pyridin-4-yl)-2-fluoro-4-(1-methyl-1H-1,2,3-triazol-4-yl)-N—((R)-piperidin-3-yl)benzamide (Example 613)

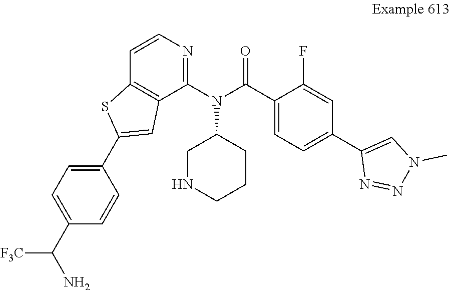 [SMILES: Cn1cc(-c2ccc(C(=O)N(c3nccc4sc(-c5ccc(C(N)C(F)(F)F)cc5)cc34)[C@@H]3CCCNC3)c(F)c2)nn1]

Example 605 was prepared according to the procedure described for Example 363 by substituting 4-pyridylboronic acid with 2,2,2-trifluoro-1-[4-(4,4,5,5-tetramethyl-1,3,2-dioxaborolan-2-yl)phenyl]ethylamine, provided the title compound hydrochloride salt as a white solid. LC-MS: [M+H⁺]=610.3. ¹H NMR (400 MHZ, CD₃OD): δ 8.32 (br d, J=5.5 Hz, 1H), 8.15 (s, 1H), 7.81 (br d, J=7.3 Hz, 3H), 7.75 (s, 1H), 7.63 (br d, J=8.0 Hz, 2H), 7.36-7.24 (m, 2H), 7.17 (br dd, J=2.6, 6.6 Hz, 1H), 4.54 (br d, J=8.0 Hz, 2H), 4.05 (s, 3H), 3.62-3.52 (m, 1H), 3.24-3.15 (m, 1H), 3.03-2.90 (m, 1H), 2.59-2.27 (m, 2H), 2.05-1.92 (m, 1H), 1.78-1.64 (m, 2H), 1.36-1.19 (m, 1H).

### Synthesis of (R)—N-(6-fluoro-8-methylisoquinolin-1-yl)-4-(1-methyl-1H-1,2,3-triazol-4-yl)-N-(piperidin-3-yl)benzamide (Example 614)

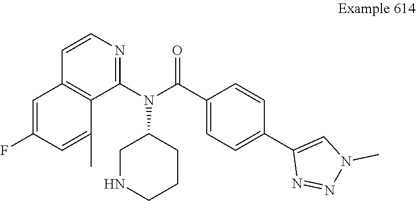 [SMILES: Cc1cc(F)cc2ccnc(N(C(=O)c3ccc(-c4cn(C)nn4)cc3)[C@@H]3CCCNC3)c12]

Example 2434 was prepared according to the procedure described for Example 111 by substituting (R)-8-methyl-N-(piperidin-3-yl)isoquinolin-1-amine with (R)-6-fluoro-8-methyl-N-(piperidin-3-yl)isoquinolin-1-amine, provided the title compound hydrochloride salt as a white solid. LC-MS: [M+H⁺]=445.2. ¹H NMR (CD₃OD, 400 MHZ): δ 8.54-8.49 (m, 1H), 8.39 (s, 1H), 7.83-7.80 (m, 1H), 7.56-7.53 (m, 1H), 7.45-7.43 (m, 2H), 7.36-7.33 (m, 1H), 7.03-7.01 (m, 2H), 4.72-4.67 (m, 1H), 4.00 (s, 3H), 3.56-3.50 (m, 1H), 3.01-2.92 (m, 2H), 2.69 (s, 3H), 2.49-2.28 (m, 2H), 1.63-1.58 (m, 2H), 1.19-1.11 (m, 1H).

### Synthesis of N-(6-chloro-8-methyl-1-isoquinolyl)-2-fluoro-N-[(3R)-3-piperidyl]-4-(triazolo[4,5-b]pyridin-3-yl)benzamide (Example 615)

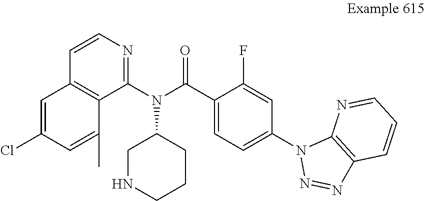 [SMILES: Cc1cc(Cl)cc2ccnc(N(C(=O)c3ccc(-n4nnc5cccnc54)cc3F)[C@@H]3CCCNC3)c12]

Example 615 was prepared according to the procedure described for Example 241 by substituting tert-butyl (3R)-3-[(6-chloro-1-isoquinolyl)amino]piperidine-1-carboxylate with tert-butyl (R)-3-((6-chloro-8-methylisoquinolin-1-yl)amino)piperidine-1-carboxylate, provided the title compound hydrochloride salt (70%) as a white solid. LC-MS: [M+H⁺]=516.3. ¹H NMR (400 MHZ, CD3OD): δ 8.77 (dd, J=1.4, 4.6 Hz, 1H), 8.54-8.46 (m, 2H), 8.08-7.98 (m, 1H), 7.89 (dd, J=1.8, 8.6 Hz, 1H), 7.73-7.66 (m, 2H), 7.54 (dd, J=4.5, 8.4 Hz, 1H), 7.52-7.48 (m, 1H), 7.15-7.09 (m, 1H), 5.14-5.04 (m, 1H), 4.07-3.99 (m, 1H), 3.88-3.80 (m, 1H), 3.47-3.39 (m, 1H), 3.09-2.96 (m, 1H), 2.92-2.85 (m, 3H), 2.05-1.89 (m, 3H), 1.68-1.53 (m, 1H).

### Synthesis of (R)—N-(3-ethylthieno[3,2-c]pyridin-4-yl)-2-fluoro-4-(5-methyl-1,3,4-thiadiazol-2-yl)-N-(piperidin-3-yl)benzamide (Example 617)

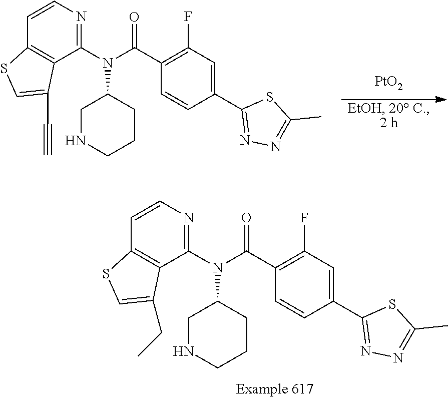 [SMILES: C#Cc1csc2ccnc(N(C(=O)c3ccc(-c4nnc(C)s4)cc3F)[C@@H]3CCCNC3)c12 ; "PtO2" ; "EtOH, 20° C.,2 h" ; CCc1csc2ccnc(N(C(=O)c3ccc(-c4nnc(C)s4)cc3F)[C@@H]3CCCNC3)c12]

A mixture of (R)—N-(3-ethynylthieno[3,2-c]pyridin-4-yl)-2-fluoro-4-(5-methyl-1,3,4-thiadiazol-2-yl)-N-(piperidin-3-yl)benzamide (40 mg, 75.21 μmol), PtO₂ (2.01 mg, 7.52 μmol) in ethyl alcohol (2 mL) was degassed and purged with H₂ for 3 times, and then the mixture was stirred at 20° C. for 2 h under H₂ atmosphere. The reaction mixture was filtered and concentrated. The residue was purified by reverse-phase Prep-HPLC to give the title compound as a white solid. LC-MS: [M+H⁺]=482.3. ¹H NMR (400 MHZ, CD₃OD): δ ppm 8.37 (d, J=5.4 Hz, 1H), 7.92 (d, J=5.5 Hz, 1H), 7.56-7.46 (m, 2H), 7.36 (dd, J=1.4, 8.1 Hz, 1H), 7.09 (d, J=7.0 Hz, 1H), 5.11-5.01 (m, 1H), 3.97 (br d, J=12.1 Hz, 1H), 3.78-3.69 (m, 1H), 3.38 (br s, 1H), 3.12-2.83 (m, 3H), 2.76 (s, 3H), 1.97-1.82 (m, 3H), 1.48 (t, J=7.3 Hz, 3H), 1.40-1.27 (m, 1H).

### Synthesis of (R)—N-(6-chloro-8-methylisoquinolin-1-yl)-2-fluoro-4-(5-(hydroxymethyl)-1-methyl-1H-1,2,3-triazol-4-yl)-N-(piperidin-3-yl)benzamide (Example 618)

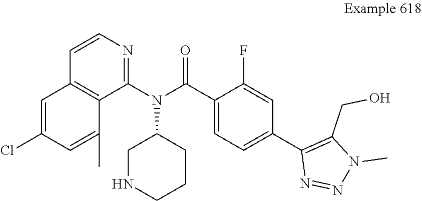 [SMILES: Cc1cc(Cl)cc2ccnc(N(C(=O)c3ccc(-c4nnn(C)c4CO)cc3F)[C@@H]3CCCNC3)c12]

Example 618 was prepared according to the procedure described for Example 816 by substituting 4-bromo-2-chloro-benzoyl chloride and 4-bromo-1-methyl-triazole with 4-bromo-2-fluoro-benzoyl chloride and (4-bromo-1-methyl-1H-1,2,3-triazol-5-yl) methanol, provided the title compound hydrochloride salt as an off-white solid. LC-MS: [M+H⁺]=509.3. ¹H NMR (400 MHz, CD₃OD): δ ppm 8.53-8.42 (m, 1H), 7.75-7.64 (m, 2H), 7.52-7.44 (m, 1H), 7.28-7.18 (m, 1H), 7.13 (br d, J=8.0 Hz, 1H), 7.00-6.89 (m, 1H), 5.15-5.03 (m, 1H), 4.60 (s, 2H), 4.10 (s, 3H), 4.05-3.97 (m, 1H), 3.90-3.79 (m, 1H), 3.45-3.39 (m, 1H), 3.09-2.98 (m, 1H), 2.92-2.81 (m, 3H), 2.81-2.69 (m, 1H), 2.28-1.89 (m, 3H), 1.63 (br dd, J=4.4, 12.1 Hz, 1H).

### Synthesis of (R)-2-fluoro-N-(6-methoxy-8-methylisoquinolin-1-yl)-4-(1-methyl-1H-1,2,3-triazol-4-yl)-N-(piperidin-3-yl)benzamide (Example 619)

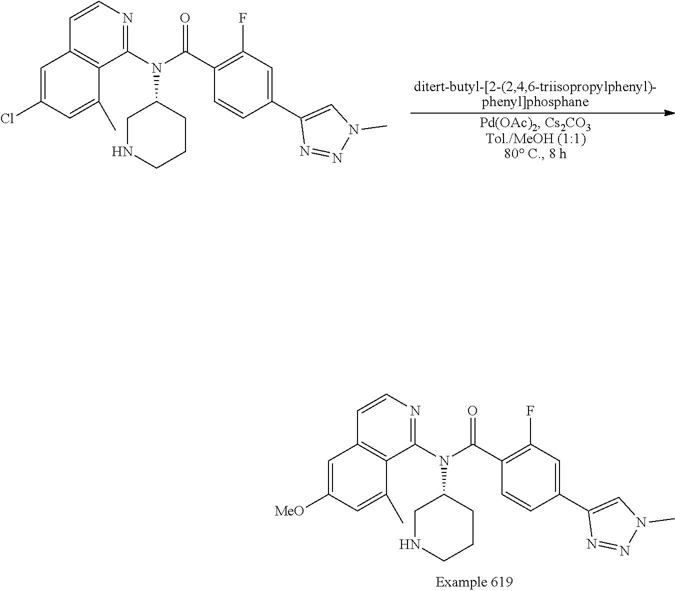

To a mixture of (R)—N-(6-chloro-8-methylisoquinolin-1-yl)-2-fluoro-4-(1-methyl-1H-1,2,3-triazol-4-yl)-N-(piperidin-3-yl)benzamide (100 mg, 208.79 μmol) and Cs₂CO₃ (102.04 mg, 313.19 μmol) in MeOH (2 mL) and toluene (2 mL) were added Pd(OAc)₂ (4.69 mg, 20.88 μmo) and ditert-butyl-[2-(2,4,6-triisopropylphenyl)phenyl] phosphane (8.87 mg, 20.88 μmol). The reaction mixture was degassed with N₂ at 20° C. Then the mixture was heated to 80° C. and stirred for 8 h under N₂ atmosphere. After evaporation and prep-HPLC purification. The combined pure fraction was lyophilized to give the title compound hydrochloride salt (37.4 mg, 37%) as an off-white solid. LC-MS: [M+H⁺]=475.3. ¹H NMR (400 MHZ, CD₃OD): δ ppm 8.39-8.31 (m, 1H), 8.22 (s, 1H), 7.84-7.74 (m, 1H), 7.34-7.28 (m, 1H), 7.27-7.18 (m, 2H), 7.17-7.12 (m, 1H), 7.06 (quin, J=7.6 Hz, 1H), 5.04-4.94 (m, 1H), 4.12-4.00 (m, 4H), 3.87-3.79 (m, 4H), 3.42 (br d, J=12.5 Hz, 1H), 3.03 (dt, J=5.1, 12.0 Hz, 1H), 2.84-2.78 (m, 3H), 2.03-1.81 (m, 3H), 1.64-1.50 (m, 1H).

### Synthesis of 2-fluoro-N-(6-fluoro-8-methyl-1-isoquinolyl)-4-(1-methyltriazol-4-yl)-N-[(3S)-3-piperidyl]benzamide (Example 620)

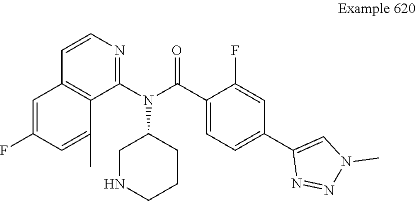 [SMILES: Cc1cc(F)cc2ccnc(N(C(=O)c3ccc(-c4cn(C)nn4)cc3F)[C@@H]3CCCNC3)c12]

Example 620 was prepared following the synthesis for example 474 by substituting tert-butyl (R)-3-aminopiperidine-1-carboxylate with tert-butyl(S)-3-aminopiperidine-1-carboxylate to afford the title compound as a white solid. LC-MS: [M+H]=463.3. ¹H NMR (400 MHZ, CD₃OD): δ ppm 1.36-1.46 (m, 1H) 1.66-1.79 (m, 3H) 2.48-2.60 (m, 2H) 2.83-2.89 (m, 3H) 2.97-3.08 (m, 2H) 3.72 (br d, J=11.51 Hz, 1H) 4.07 (s, 3H) 4.69-4.77 (m, 1H) 4.76-4.77 (m, 1H) 6.85-6.92 (m, 1H) 7.15 (dd, J=8.13, 1.50 Hz, 1H) 7.22-7.32 (m, 3H) 7.62-7.66 (m, 1H) 8.15 (s, 1H) 8.36-8.41 (m, 1H).

### Synthesis of(S)—N-(6-chloro-8-methylisoquinolin-1-yl)-2-fluoro-4-(1-methyl-1H-1,2,3-triazol-4-yl)-N-(piperidin-3-yl)benzamide (Example 621)

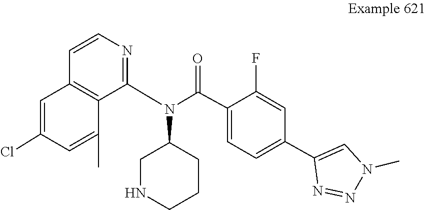 [SMILES: Cc1cc(Cl)cc2ccnc(N(C(=O)c3ccc(-c4cn(C)nn4)cc3F)[C@H]3CCCNC3)c12]

Example 621 was prepared according to the procedure described for Example 444 by substituting tert-butyl (3R)-3-[(6-chloro-8-methyl-1-isoquinolyl)amino]piperidine-1-carboxylate with tert-butyl (3S)-3-[(6-chloro-8-methyl-1-isoquinolyl)amino]piperidine-1-carboxylate, provided the title compound as an off-white solid. LC-MS: [M+H⁺]=479.3. ¹H NMR (400 MHZ, CD₃OD): δ ppm 8.47-8.38 (m, 1H), 8.15 (s, 1H), 7.67 (d, J=1.6 Hz, 1H), 7.63-7.57 (m, 1H), 7.45 (s, 1H), 7.26 (dd, J=1.5, 11.3 Hz, 1H), 7.20-7.12 (m, 1H), 6.94-6.83 (m, 1H), 4.80-4.56 (m, 1H), 4.07 (s, 3H), 3.71 (br d, J=11.9 Hz, 1H), 3.39-3.34 (m, 1H), 3.05-2.96 (m, 1H), 2.89-2.79 (m, 3H), 2.58-2.46 (m, 1H), 1.97-1.61 (m, 3H), 1.47-1.31 (m, 1H).

### Synthesis of (R)-6-(5-methyl-1,3,4-thiadiazol-2-yl)-N-(8-methylisoquinolin-1-yl)-N-(piperidin-3-yl)nicotinamide (Example 622)

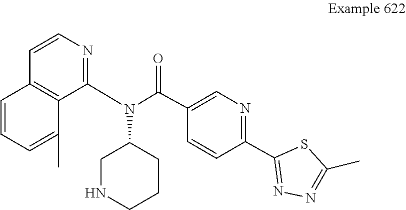 [SMILES: Cc1nnc(-c2ccc(C(=O)N(c3nccc4cccc(C)c34)[C@@H]3CCCNC3)cn2)s1]

Example 622 was prepared according to the procedure described for Example 629 by substituting tert-butyl (R)-3-((3-methylthieno[3,2-c]pyridin-4-yl)amino)piperidine-1-carboxylate with tert-butyl (R)-3-((8-methylisoquinolin-1-yl)amino)piperidine-1-carboxylate to afford the title compound as an-off-white solid. LC-MS: [M+H⁺]=445.3. ¹H NMR (CD₃OD, 400 MHZ): δ=8.55-8.54 (m, 1H), 8.22-8.21 (m, 1H), 7.87-7.83 (m, 2H), 7.72-7.65 (m, 1H), 7.57-7.51 (m, 2H), 7.49-7.45 (m, 1H), 5.14-5.12 (m, 1H), 3.97-3.92 (m, 1H), 3.84-3.78 (m, 1H), 3.41-3.40 (m, 1H), 3.05-2.82 (m, 1H), 2.83-2.75 (m, 6H), 1.96-1.90 (m, 3H), 1.52-1.50 (m, 1H).

### Synthesis of 2-fluoro-4-((4-((R)-3-hydroxypiperidin-1-yl)pyrimidin-2-yl)amino)-N-(8-methylisoquinolin-1-yl)-N—((R)-piperidin-3-yl)benzamide (Example 623)

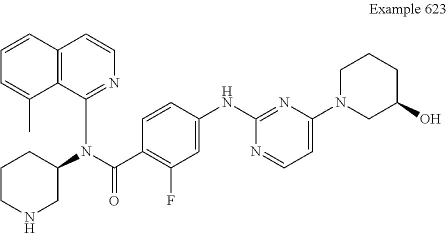 [SMILES: Cc1cccc2ccnc(N(C(=O)c3ccc(Nc4nccc(N5CCC[C@@H](O)C5)n4)cc3F)[C@@H]3CCCNC3)c12]

Example 623 was synthesized following the procedure for Example 131 by substituting 3-amino-1-methylpyridin-2 (1H)-one with (R)-1-(2-aminopyrimidin-4-yl)piperidin-3-ol to afford the title compound as an off-white solid. LC-MS: [M+H⁺]=556. ¹H NMR (CD₃OD, 400 MHZ): δ ppm 8.43-8.40 (m, 1H), 7.74-7.63 (m, 3H), 7.53-7.45 (m, 2H), 7.12-7.05 (m, 1H), 6.83-6.75 (m, 2H), 6.60-6.56 (m, 1H), 5.08-4.92 (m, 1H), 3.99-3.90 (m, 2H), 3.83-3.77 (m, 1H), 3.68-3.57 (m, 3H), 3.41-3.38 (m, 1H), 3.01-2.99 (m, 1H), 2.81 (s, 3H), 2.71-2.66 (m, 1H), 1.97-1.94 (m, 5H), 1.77-1.74 (m, 1H), 1.61-1.55 (m, 2H).

### Synthesis of 2-fluoro-4-((4-((S)-3-hydroxypiperidin-1-yl)pyrimidin-2-yl)amino)-N-(8-methylisoquinolin-1-yl)-N—((R)-piperidin-3-yl)benzamide (Example 624)

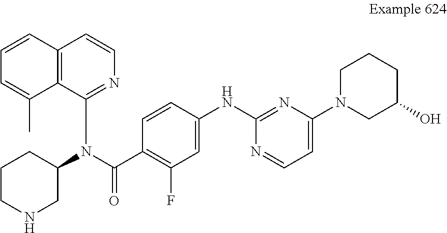 [SMILES: Cc1cccc2ccnc(N(C(=O)c3ccc(Nc4nccc(N5CCC[C@H](O)C5)n4)cc3F)[C@@H]3CCCNC3)c12]

Example 624 was synthesized following the procedure for Example 131 by substituting 3-amino-1-methylpyridin-2(1H)-one with(S)-1-(2-aminopyrimidin-4-yl)piperidin-3-ol to afford the title compound as an off-white solid. LC-MS: [M+H⁺]=556.2. 1H NMR (CD₃OD, 400 MHZ): δ 8.44-8.40 (m, 1H), 7.75-7.70 (m, 2H), 7.68-7.63 (m, 1H), 7.53-7.45 (m, 2H), 7.13-7.05 (m, 1H), 6.84-6.78 (m, 2H), 6.61-6.56 (m, 1H), 5.08-5.03 (m, 1H), 3.95-3.80 (m, 4H), 3.72-3.56 (m, 4H), 3.41-3.38 (m, 1H), 3.02-2.99 (m, 1H), 2.81 (s, 3H), 2.71-2.67 (m, 1H), 1.97-1.92 (m, 4H), 1.61-1.53 (m, 2H).

### Synthesis of 2-fluoro-4-((4-((S)-3-hydroxypyrrolidin-1-yl)pyrimidin-2-yl)amino)-N-(8-methylisoquinolin-1-yl)-N—((R)-piperidin-3-yl)benzamide (Example 625)

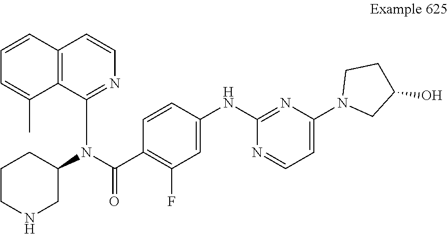 [SMILES: Cc1cccc2ccnc(N(C(=O)c3ccc(Nc4nccc(N5CC[C@H](O)C5)n4)cc3F)[C@@H]3CCCNC3)c12]

Example 625 was synthesized following the procedure for Example 131 by substituting 3-amino-1-methylpyridin-2 (1H)-one with(S)-1-(2-aminopyrimidin-4-yl)pyrrolidin-3-ol to afford the title compound hydrochloride salt as a white solid. LC-MS: [M+H⁺]=542.2. ¹H NMR (CD₃OD, 400 MHZ): δ ppm 8.43-8.40 (m, 1H), 7.75-7.72 (m, 2H), 7.70-7.65 (m, 1H), 7.52-7.44 (m, 2H), 7.31-7.24 (m, 1H), 6.82-6.79 (m, 2H), 6.37-6.31 (m, 1H), 5.08-5.02 (m, 1H), 4.60-4.59 (m, 1H), 3.98-3.95 (m, 1H), 3.80-3.79 (m, 1H), 3.71-3.68 (m, 2H), 3.58-3.53 (m, 2H), 3.41-3.38 (m, 1H), 3.01-2.99 (m, 1H), 2.81 (s, 3H), 2.70-2.67 (m, 1H), 2.23-2.18 (m, 2H), 1.94-1.88 (m, 2H), 1.57-1.54 (m, 1H).

### Synthesis of (R)-2-fluoro-N-(5-fluoro-8-methylisoquinolin-1-yl)-N-(piperidin-3-yl)-4-(pyrazolo[1,5-a]pyrimidin-3-yl)benzamide (Example 626)

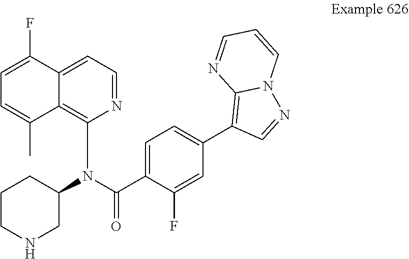 [SMILES: Cc1ccc(F)c2ccnc(N(C(=O)c3ccc(-c4cnn5cccnc45)cc3F)[C@@H]3CCCNC3)c12]

Example 626 was made following the procedure for Example 761 by substituting 4-bromo-1-methyl-1H-1,2,3-triazole with 3-bromopyrazolo[1,5-a]pyrimidine to yield (R)-2-fluoro-N-(6-fluoro-8-methylisoquinolin-1-yl)-N-(piperidin-3-yl)-4-(pyrazolo[1,5-a]pyrimidin-3-yl)benzamide hydrochloride salt as an off-white solid. LC-MS: [M+H⁺]=499.65. ¹H NMR (CD₃OD, 400 MHZ): δ 8.89-8.87 (m, 1H), 8.60-8.54 (m, 1H), 8.55 (d, J=5.6 Hz, 1H), 8.43 (s, 1H), 7.93-7.89 (m, 1H), 7.73-7.70 (m, 1H), 7.47-7.42 (m, 2H), 7.26-7.22 (m, 1H), 7.04-7.02 (m, 1H), 6.82-6.78 (m, 1H), 5.09-5.03 (m, 1H), 4.00-3.98 (m, 1H), 3.86-3.80 (m, 1H), 3.48-3.39 (m, 1H), 3.03-3.00 (m, 1H), 2.80 (s, 3H), 1.98-1.89 (m, 2H), 1.60-1.56 (m, 1H).

### Synthesis of (R)-2-fluoro-N-(6-fluoro-8-methylisoquinolin-1-yl)-N-(piperidin-3-yl)-4-(pyrazolo[1,5-a]pyrimidin-3-yl)benzamide (Example 627)

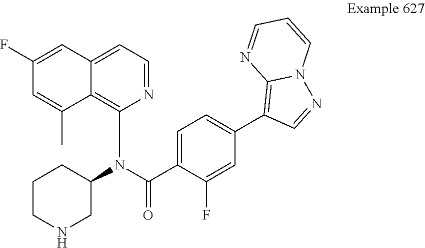 [SMILES: Cc1cc(F)cc2ccnc(N(C(=O)c3ccc(-c4cnn5cccnc45)cc3F)[C@@H]3CCCNC3)c12]

Example 627 was prepared following the synthesis for Example 833 by substituting 2-bromo-5-methyl-1,3,4-thiadiazole with 3-bromopyrazolo[1,5-a]pyrimidine to afford the title compound as an off-white solid. LC-MS: [M+H⁺]=499.1. ¹H NMR (CD₃OD, 400 MHz): δ ppm 8.89-8.87 (m, 1H), 8.60-8.58 (m, 1H), 8.44-8.40 (m, 2H), 7.74-7.67 (m, 2H), 7.48-7.45 (m, 1H), 7.33-7.28 (m, 2H), 7.04-7.02 (m, 1H), 6.83-6.78 (m, 1H), 5.10-5.03 (m, 1H), 4.00-3.96 (m, 1H), 3.86-3.80 (m, 1H), 3.42-3.38 (m, 1H), 3.05-2.99 (m, 1H), 2.85 (s, 3H), 2.71-2.70 (m, 1H), 1.98-1.89 (m, 2H), 1.62-1.57 (m, 1H).

### Synthesis of (R)-2-methyl-4-(5-methyl-1,3,4-thiadiazol-2-yl)-N-(8-methylisoquinolin-1-yl)-N-(piperidin-3-yl)benzamide (Example 628)

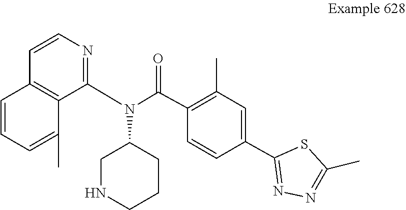 [SMILES: Cc1nnc(-c2ccc(C(=O)N(c3nccc4cccc(C)c34)[C@@H]3CCCNC3)c(C)c2)s1]

Example 628 was prepared according to the procedure described for Example 622 by substituting methyl 5-(chlorocarbonyl)picolinate with methyl 4-(chlorocarbonyl)-3-methylbenzoate to afford the title compound as an off-white solid. LC-MS: [M+H⁺]=459.0. ¹H NMR (CD₃OD, 400 MHZ): δ ppm 8.30-8.28 (m, 1H), 7.67-7.63 (m, 2H), 7.57-7.53 (m, 2H), 7.45 (d, J=8.4 Hz, 1H), 7.19 (d, J=8.4 Hz, 1H), 6.94 (d, J=8.0 Hz, 1H), 5.05-5.00 (m, 1H), 4.14-4.11 (m, 1H), 3.76-3.70 (m, 1H), 3.41-3.38 (m, 1H), 3.15-2.99 (m, 1H), 2.97 (s, 3H), 2.86-2.85 (m, 1H), 2.71 (s, 3H), 2.43 (s, 3H), 1.96-1.91 (m, 2H), 1.49-1.45 (m, 1H).

### Synthesis of 6-(5-methyl-1,3,4-thiadiazol-2-yl)-N-(3-methylthieno[3,2-c]pyridin-4-yl)-N-[(3R)-3-piperidyl]pyridine-3-carboxamide (Example 629)

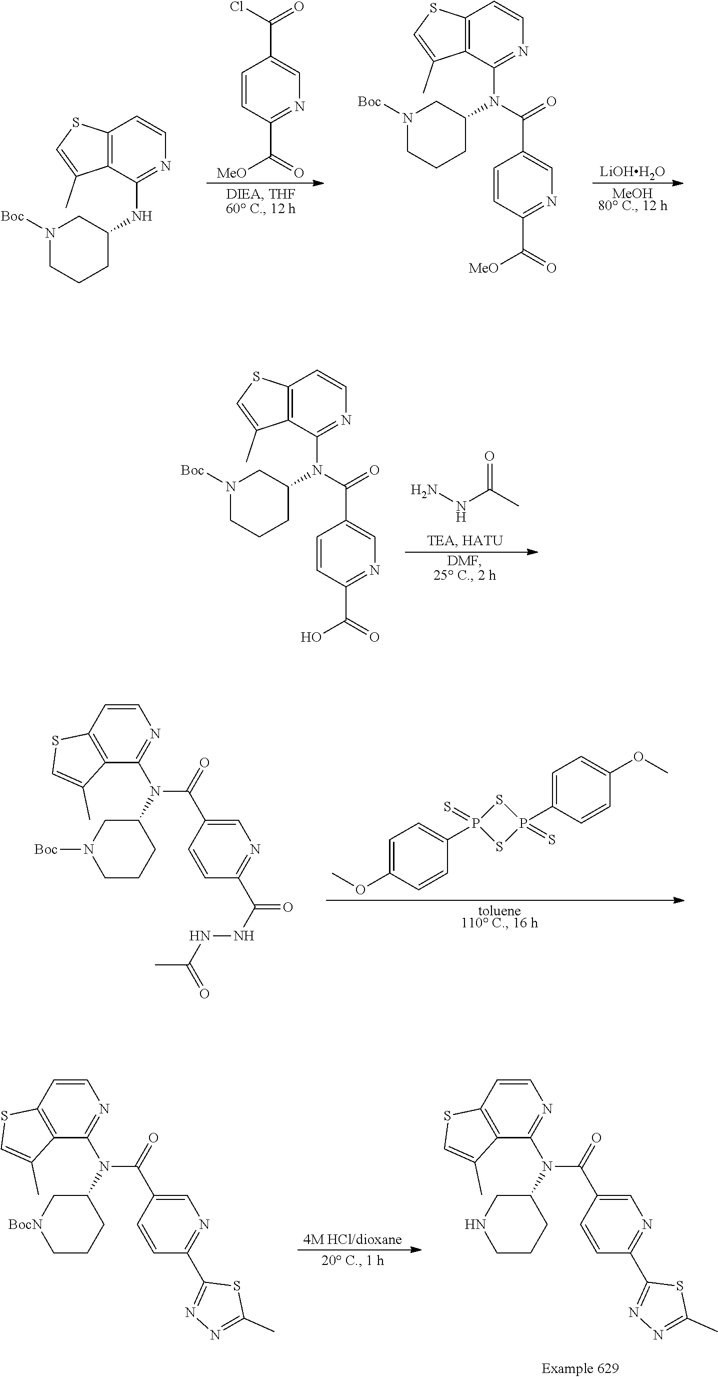

### Step 1:

A mixture of tert-butyl (3R)-3-[(3-methylthieno[3,2-c]pyridin-4-yl)amino]piperidine-1-carboxylate (500 mg, 1.44 mmol), methyl 5-chlorocarbonylpyridine-2-carboxylate (574.41 mg, 2.88 mmol), and TEA (728.03 mg, 7.19 mmol) in toluene (10 mL) was degassed with N₂ at 25° C., and then the mixture was stirred at 90° C. for 12 h under N₂. The mixture was concentrated. The residue was purified by column chromatography (SiO₂, Petroleum ether/EtOAc=9/1 to 5/1) to give methyl 5-[[(3R)-1-tert-butoxycarbonyl-3-piperidyl]-(3-methylthieno[3,2-c]pyridin-4-yl)carba-moyl]pyridine-2-carboxylate (700 mg, 95%) as a yellow solid.

To a solution of methyl 5-[[(3R)-1-tert-butoxycarbonyl-3-piperidyl]-(3-methylthieno-[3,2-c]pyridin-4-yl)carbamoyl]pyridine-2-carboxylate (0.9 g, 1.76 mmol) in THF (10 mL) and H₂O (10 mL) was added LiOH·H₂O (221.88 mg, 5.29 mmol). The mixture was stirred at 20° C. for 4 h. The mixture was acidified by adding 1M hydrochloric acid dropwise at 20° C. to pH 3. Then the resulting mixture was extracted with EtOAc (3×100 mL). The combined organic phase was washed with brine (200 mL) and dried over Na₂SO₄. The mixture was concentrated to give 5-[[(3R)-1-tert-butoxycarbonyl-3-piperidyl]-(3-methylthieno[3,2-c]pyridin-4-yl)carbamoyl]pyridine-2-carboxylic acid (800 mg, 91%) as a yellow solid, which was used in the next step directly.

### Step 3:

A mixture of 5-[[(3R)-1-tert-butoxycarbonyl-3-piperidyl]-(3-methylthieno[3,2-c]pyridin-4-yl)carbamoyl]pyridine-2-carboxylic acid (700 mg, 1.41 mmol), acetohydrazide (208.86 mg, 2.82 mmol), TEA (285.28 mg, 2.82 mmol), and HATU (1.07 g, 2.82 mmol) in THF (10 mL) was stirred for 2 h at 25° C. The mixture was basified by adding 1 N Na₂CO₃ to pH 9. Then the resulting mixture was extracted with EtOAc (3×100 mL). The combined organic phase was washed with brine (200 mL), dried over Na₂SO₄, and concentrated. The residue was purified by prep-HPLC to give tert-butyl (3R)-3-[[6-(acetamidocarbamoyl)-pyridine-3-carbonyl]-(3-methylthieno[3,2-c]pyridin-4-yl)amino]piperidine-1-carboxylate (400 mg, 45%) as a yellow solid.

### Step 4:

A mixture of tert-butyl (3R)-3-[[6-(acetamidocarbamoyl)pyridine-3-carbonyl]-(3-methylthieno[3,2-c]pyridin-4-yl)amino]piperidine-1-carboxylate (280 mg, 506.66 μmol) and 2,4-bis(4-methoxyphenyl)-2,4-dithioxo-1,3,2,4-dithiadiphosphetane (409.85 mg, 1.01 mmol) in toluene (5 mL) was degassed with N₂ at 25° C., and then the mixture was heated to 60° C. and stirred for 1 h under N₂. The mixture was concentrated. The residue was purified by prep-TLC (SiO₂, Petroleum ether/EtOAc=2:1) to give tert-butyl (3R)-3-[[6-(5-methyl-1,3,4-thiadiazol-2-yl)pyridine-3-carbonyl]-(3-methylthieno[3,2-c]pyridin-4-yl)amino]piperidine-1-carboxylate (110 mg, 39%) as a yellow solid.

### Step 5:

The title compound hydrochloride salt was obtained as an off-white solid by deprotection Method A used in Example 97. LC-MS: [M+H⁺]=451.2. ¹H NMR (400 MHZ, CD₃OD): δ=8.48 (d, J=5.5 Hz, 1H), 8.36 (d, J=1.6 Hz, 1H), 8.01 (d, J=5.4 Hz, 1H), 7.94 (d, J=8.3 Hz, 1H), 7.70 (dd, J=2.3, 8.3 Hz, 1H), 7.39 (d, J=1.0 Hz, 1H), 5.20-5.10 (m, 1H), 3.98-3.91 (m, 1H), 3.79-3.70 (m, 1H), 3.40-3.35 (m, 1H), 2.99-2.88 (m, 1H), 2.77 (s, 3H), 2.50 (d, J=0.9 Hz, 3H), 1.95-1.79 (m, 3H), 1.37-1.25 (m, 1H).

### Synthesis of (R)-4-(4-(methoxymethyl)-1H-1,2,3-triazol-1-yl)-N-(3-methylthieno[3,2-c]pyridin-4-yl)-N-(piperidin-3-yl)benzamide (Example 630)

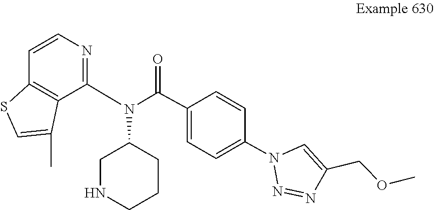 [SMILES: COCc1cn(-c2ccc(C(=O)N(c3nccc4scc(C)c34)[C@@H]3CCCNC3)cc2)nn1]

Example 630 was prepared according to the procedure described for Example 632 by substituting 5-azidopyridine-2-carboxylate and tert-butyl (3R)-3-[(6-chloro-8-methyl-1-isoquinolyl) amino] piperidine-1-carboxylate with methyl 4-azidobenzoate and tert-butyl (3R)-3-[(3-methylthieno[3,2-c]pyridin-4-yl)amino]piperidine-1-carboxylate, provided the title compound hydrochloride salt as a white solid. LC-MS: [M+H⁺]=463.3. ¹H NMR (400 MHz, CD₃OD): δ ppm 8.51 (br d, J=5.9 Hz, 2H), 8.21-8.06 (m, 1H), 7.65 (br d, J=8.4 Hz, 2H), 7.56-7.38 (m, 3H), 5.12 (br s, 1H), 4.57 (s, 2H), 3.97 (br d, J=10.9 Hz, 1H), 3.87-3.70 (m, 1H), 3.38 (s, 4H), 3.07-2.88 (m, 1H), 2.68-2.44 (m, 3H), 2.02-1.68 (m, 3H), 1.46-1.23 (m, 1H).

### Synthesis of (R)—N-(6-chloro-8-methylisoquinolin-1-yl)-6-(4-(methoxymethyl)-1H-1,2,3-triazol-1-yl)-N-(piperidin-3-yl)nicotinamide (Example 631)

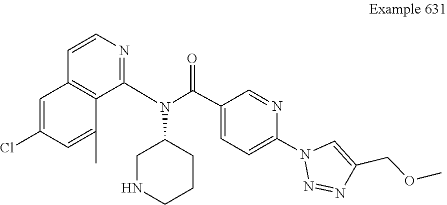 [SMILES: COCc1cn(-c2ccc(C(=O)N(c3nccc4cc(Cl)cc(C)c34)[C@@H]3CCCNC3)cn2)nn1]

Example 631 was prepared according to the procedure described for Example 632 by substituting methyl 5-bromopyridine-2-carboxylate with methyl 6-bromopyridine-3-carboxylate, provided the title compound hydrochloride salt as a white solid. LC-MS: [M+H⁺]=492.3. ¹H NMR (400 MHZ, CD₃OD): δ ppm 8.63-8.49 (m, 2H), 8.17-8.12 (m, 1H), 7.87-7.75 (m, 3H), 7.72-7.65 (m, 1H), 7.47-7.42 (m, 1H), 5.19-5.04 (m, 1H), 4.55 (s, 2H), 3.98 (br d, J=12.1 Hz, 1H), 3.84-3.74 (m, 1H), 3.46-3.36 (m, 4H), 3.05-2.94 (m, 1H), 2.83-2.73 (m, 3H), 2.22-1.82 (m, 3H), 1.49 (dq, J=5.6, 11.9 Hz, 1H).

### Synthesis of (R)—N-(6-chloro-8-methylisoquinolin-1-yl)-5-(4-(methoxymethyl)-1H-1,2,3-triazol-1-yl)-N-(piperidin-3-yl)picolinamide (Example 632)

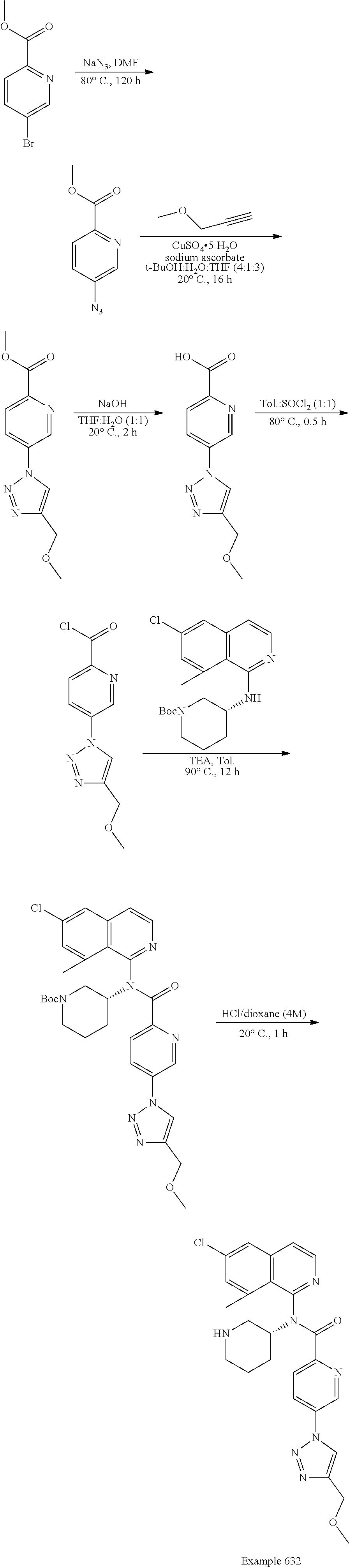

### Step 1:

To a solution of methyl 5-bromopyridine-2-carboxylate (3 g, 13.89 mmol) in DMF (30 mL) was added NaN₃ (1.81 g, 27.77 mmol) under N₂ at 25° C. The mixture was heated to 80° C. and stirred for 120 h. The reaction mixture was filtered and concentrated under reduced pressure to give a residue to give methyl 5-azidopyridine-2-carboxylate (2.5 g, 70%) as a white solid.

### Step 2:

To a solution of methyl 5-azidopyridine-2-carboxylate (2.4 g, 13.47 mmol) and 3-methoxyprop-1-yne (944.24 mg, 13.47 mmol, 1.11 mL) in t-BuOH (20 mL), H₂O (10 mL) and THF (15 mL) were added CuSO₄·5H₂O (336.37 mg, 1.35 mmol) and sodium ascorbate (266.89 mg, 1.35 mmol). The mixture was stirred at 20° C. for 16 h. The reaction mixture was filtered and concentrated under reduced pressure to give a residue, which was purified by column chromatography (SiO₂, Petroleum ether/Tetrahydrofuran=10/1 to 8/1) to give methyl 5-[4-(methoxymethyl) triazol-1-yl] pyridine-2-carboxylate (1.5 g, 45%) as a white solid.

### Step 3:

A mixture of methyl 5-[4-(methoxymethyl) triazol-1-yl] pyridine-2-carboxylate (1.3 g, 5.24 mmol) and NaOH (628.43 mg, 15.71 mmol) in THF (13 mL) and water (13 mL) was stirred at room temperature for 2 h. The reaction mixture was diluted with water (20 mL) and was acidified by adding 1 N HCl dropwise at 0° C. to pH=2. The reaction mixture was extracted with EtOAc (7×10 mL). The combined organic layer was concentrated under reduced pressure and dried to give 5-[4-(methoxymethyl) triazol-1-yl] pyridine-2-carboxylic acid (1.1 g, 78%) as a white solid.

### Steps 4-6:

Example 632 was prepared according to the procedure described for Example 444 by substituting 2-fluoro-4-(1-methyltriazol-4-yl) benzoic acid with 5-[4-(methoxymethyl) triazol-1-yl] pyridine-2-carboxylic acid, provided the title compound hydrochloride salt as a white solid. LC-MS: [M+H⁺]=492.3. ¹H NMR (400 MHZ, CD₃OD): δ ppm 8.50 (s, 1H), 8.43-8.29 (m, 3H), 8.15 (d, J=8.6 Hz, 1H), 7.97-7.92 (m, 1H), 7.90 (d, J=5.8 Hz, 1H), 7.63-7.50 (m, 1H), 5.07-4.92 (m, 1H), 4.55 (s, 2H), 4.09-3.95 (m, 1H), 3.51 (br t, J=11.4 Hz, 1H), 3.45-3.33 (m, 4H), 2.97-2.79 (m, 4H), 1.96-1.74 (m, 3H), 1.19-0.95 (m, 1H).

### Synthesis of (R)-4-(5-(2-methoxyethyl)-1,3,4-thiadiazol-2-yl)-N-(3-methylthieno[3,2-c]pyridin-4-yl)-N-(piperidin-3-yl)benzamide (Example 633)

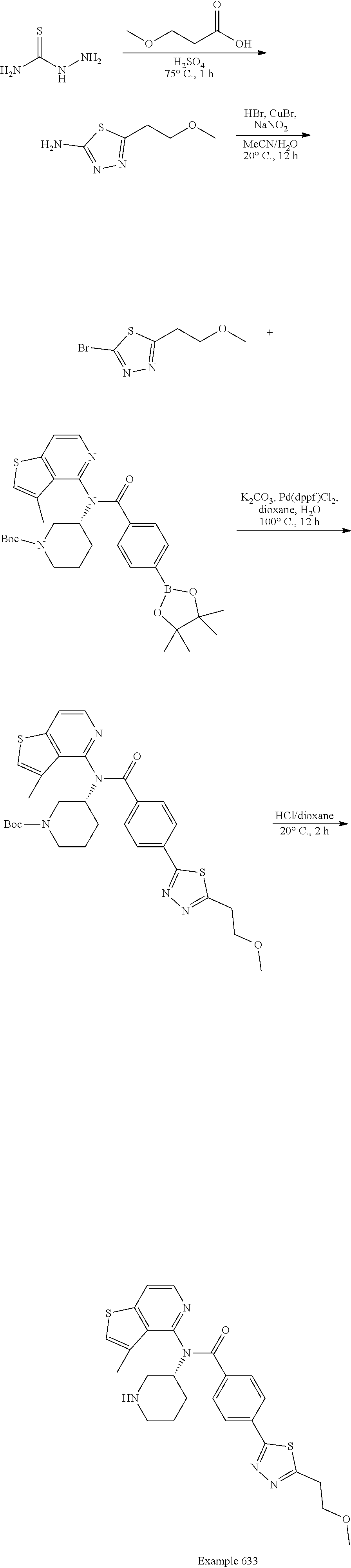

### Step 1:

To a solution of aminothiourea (1 g, 10.97 mmol) in H₂SO₄ (10 mL) was added 3-methoxypropanoic acid (1.14 g, 10.97 mmol, 1.03 mL) at 20° C. The mixture was stirred at 75° C. for 1 h. The mixture was adjusted to pH 7 with aqueous sodium carbonate (50 mL) and concentrated. The residue was dissolved in methanol (10 mL), filtered, and the filtrate was concentrated to give 5-(2-methoxyethyl)-1,3,4-thiadiazol-2-amine (3 g, 68%) as a brown oil. LC-MS: [M+H⁺]=160.2

### Step 2:

To a solution of 5-(2-methoxyethyl)-1,3,4-thiadiazol-2-amine (3.00 g, 7.54 mmol) in MeCN (30 mL) and H₂O at 0° C. (5 mL) was added HBr (15.25 g, 75.37 mmol, 40%) and then copper bromide (1.30 g, 9.04 mmol). Then NaNO₂ (624.08 mg, 9.04 mmol) was added to the mixture at 0° C. The mixture was stirred at 20° C. for 12 hours, after which it was poured into water (50 mL) and extracted with EtOAc (2×50 mL). The combined organic phase was washed with brine (50 mL), dried with anhydrous Na₂SO₄, filtered, and concentrated. The residue was purified by prep-HPLC and the fractions containing product were lyophilized to give 2-bromo-5-(2-methoxyethyl)-1,3,4-thiadiazole (0.2 g, 11%) as an off-white solid. LC-MS: [M+H⁺]=222.7.

### Step 3:

To a solution of tert-butyl (3R)-3-[(3-methylthieno[3,2-c]pyridin-4-yl)-[4-(4,4,5,5-tetramethyl-1,3,2-dioxaborolan-2-yl)benzoyl]amino]piperidine-1-carboxylate (0.2 g, 277.04 μmol) and 2-bromo-5-(2-methoxyethyl)-1,3,4-thiadiazole (97.59 mg, 415.56 μmol) in dioxane (2 mL) and H₂O (0.5 mL) were added cyclopentyl(diphenyl)phosphane dichloropalladium iron (20.27 mg, 27.70 μmol) and potassium acetate (81.57 mg, 831.11 μmol) at 20° C. Then the mixture was stirred at 100° C. for 12 h under N₂. The mixture was poured into water (20 mL) and extracted with EtOAc (2×20 mL). The combined organic phase was washed with brine (20 mL), dried with anhydrous Na₂SO₄, filtered, and concentrated. The residue was purified by prep-HPLC and the pure fraction was lyophilized to give tert-butyl (3R)-3-[[4-[5-(2-methoxyethyl)-1,3,4-thiadiazol-2-yl]benzoyl]-(3-methylthieno[3,2-c]pyridin-4-yl)amino]piperidine-1-carboxylate (120 mg, 69%) as an off-white solid. LC-MS: [M+H⁺]=594.2.

### Step 4:

The title compound hydrochloride salt was obtained as an off-white solid by deprotection Method A used in Example 97. LC-MS: [M+H⁺]=494.3 ¹H NMR (400 MHZ, CD₃OD): δ ppm 1.24-1.38 (m, 1H) 1.81 (br d, J=12.51 Hz, 1H) 1.85-1.97 (m, 2H) 2.48-2.61 (m, 3H) 2.93 (br d, J=4.00 Hz, 1H) 3.33-3.37 (m, 5H) 3.38 (br s, 1H) 3.71 (t, J=5.82 Hz, 2H) 3.76 (d, J=11.51 Hz, 1H) 3.88-3.96 (m, 1H) 5.07-5.21 (m, 1H) 7.27-7.39 (m, 3H) 7.66 (d, J=8.50 Hz, 2H) 7.98 (d, J=5.50 Hz, 1H) 8.45 (d, J=5.38 Hz, 1H).

### Synthesis of (R)-2-fluoro-4-(5-(2-methoxyethyl)-1,3,4-thiadiazol-2-yl)-N-(3-methylthieno[3,2-c]pyridin-4-yl)-N-(piperidin-3-yl)benzamide (Example 634)

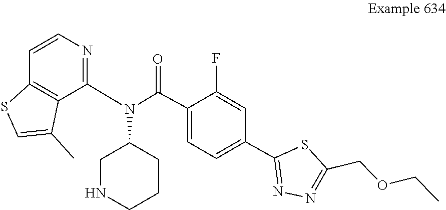 [SMILES: CCOCc1nnc(-c2ccc(C(=O)N(c3nccc4scc(C)c34)[C@@H]3CCCNC3)c(F)c2)s1]

Example 634 was prepared according to the procedure described for Example 127 by substituting tert-butyl (R)-3-((8-methylisoquinolin-1-yl)amino)piperidine-1-carboxylate with tert-butyl (R)-3-((3-methylthieno[3,2-c]pyridin-4-yl)amino)piperidine-1-carboxylate and 2-bromo-5-methyl-1,3,4-thiadiazole with 2-bromo-5-(2-methoxyethyl)-1,3,4-thiadiazole, provided the title compound as brown solid. LC-MS: [M+H]=512.3. ¹H NMR (400 MHZ, DMSO-d₆): δ ppm 9.42-8.94 (m, 2H), 8.40-8.31 (m, 1H), 8.04 (br d, J=5.5 Hz, 1H), 7.71-7.56 (m, 2H), 7.50-7.39 (m, 1H), 7.16-7.03 (m, 1H), 5.00-4.87 (m, 1H), 3.80-3.70 (m, 1H), 3.64 (br t, J=5.8 Hz, 2H), 3.50-3.39 (m, 1H), 3.33-3.29 (m, 2H), 3.27-3.20 (m, 3H), 2.61 (br s, 2H), 2.58-2.53 (m, 3H), 1.87-1.67 (m, 3H), 1.14-0.99 (m, 1H).

### Synthesis of (R)-4-(6,7-dihydro-4H-[1,2,3]triazolo[5,1-c][1,4]oxazin-3-yl)-2-fluoro-N-(3-methylthieno[3,2-c]pyridin-4-yl)-N-(piperidin-3-yl)benzamide (Example 635)

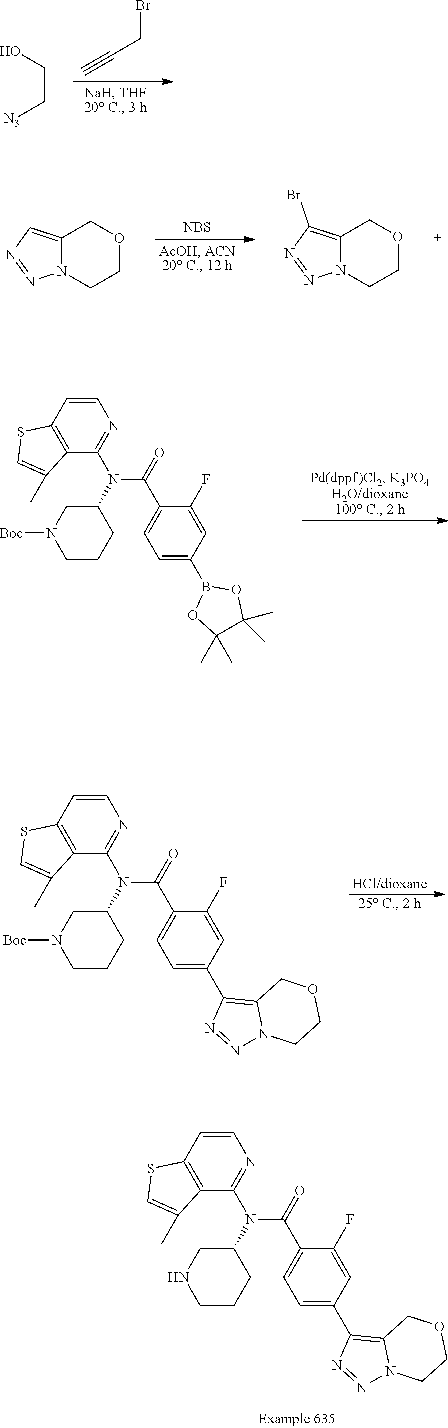

### Step 1:

To a solution of 2-azidoethanol (0.8 g, 9.19 mmol), 3-bromoprop-1-yne (1.64 g, 11.02 mmol) in tetrahydrofuran (10 mL) at 0° C. was added NaH (440.97 mg, 11.02 mmol, 60%). The mixture was stirred at 20° C. for 2 h under N₂ atmosphere. The reaction mixture was quenched by addition water (2 mL) and extracted with EtOAc (2×5 mL). The combined organic layer was washed with aqueous NaCl (10 mL), dried over Na₂SO₄, filtered and. The mixture of 3-(2-azidoethoxy) prop-1-yne (0.8 g, 6.39 mmol) in toluene (10 mL) was degassed with N₂, and then the mixture was stirred at 80° C. for 12 h. The reaction mixture was quenched by addition water (2 mL) at 0° C., and then extracted with EtOAc (2×5 mL). The combined organic layer was washed with brine, dried over Na₂SO₄, filtered and concentrated. The residue was purified by column chromatography (SiO₂, Petroleum ether/EtOAc=2/1 to 1/1) to give 6, 7-dihydro-4H-triazolo [5, 1-c] [1,4]oxazine (0.65 g, 81%) as a white solid.

### Step 2:

To a solution of 6,7-dihydro-4H-triazolo[5,1-c][1,4]oxazine (0.4 g, 3.20 mmol) in acetonitrile (4 mL) were added NBS (853.45 mg, 4.80 mmol) and AcOH (95.98 mg, 1.60 mmol) at 0° C. The mixture was stirred at 25° C. for 12 h. The reaction mixture was filtered and the filtrate was concentrated. The residue was purified by prep-HPLC to give 3-bromo-6,7-dihydro-4H-triazolo[5,1-c][1,4]oxazine (0.2 g, 30%) as a white solid.

### Steps 3-5:

Example 635 was prepared according to the procedure described for Example 699 by substituting 2-bromo-5-methyl-1,3,4-oxadiazole with 3-bromo-6,7-dihydro-4H-triazolo[5,1-c][1,4]oxazine, provided the title compound hydrochloride salt as a white solid. LC-MS: [M+H⁺]=493.3. ¹H NMR (400 MHZ, CD₃OD): δ ppm 8.41 (d, J=5.6 Hz, 1H), 8.02 (d, J=5.5 Hz, 1H), 7.51 (s, 1H), 7.27-7.13 (m, 2H), 7.05 (dd, J=1.1, 8.2 Hz, 1H), 5.12-5.02 (m, 1H), 4.99 (s, 2H), 4.43 (t, J=5.1 Hz, 2H), 4.10 (t, J=5.1 Hz, 2H), 4.04-3.94 (m, 1H), 3.83-3.67 (m, 1H), 3.38 (br d, J=12.6 Hz, 1H), 2.95 (br dd, J=4.4, 8.1 Hz, 1H), 2.61 (s, 3H), 2.00-1.76 (m, 3H), 1.41-1.23 (m, 1H).

### Synthesis of (R)—N-(6-chloro-8-methylisoquinolin-1-yl)-2-fluoro-4-(5-(2-methoxyethyl)-1-methyl-1H-1,2,3-triazol-4-yl)-N-(piperidin-3-yl)benzamide (Example 636)

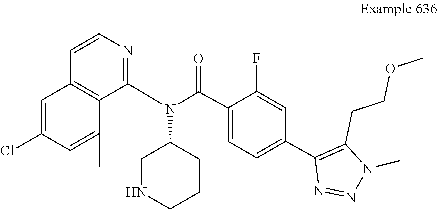 [SMILES: COCCc1c(-c2ccc(C(=O)N(c3nccc4cc(Cl)cc(C)c34)[C@@H]3CCCNC3)c(F)c2)nnn1C]

Example 636 was prepared according to the procedure described for Example 816 by substituting 4-bromo-2-chloro-benzoyl chloride and 4-bromo-1-methyl-triazole with 2-fluoro-4-iodo-benzoyl chloride and 4-bromo-5-(2-methoxyethyl)-1-methyl-triazole, provided the title compound hydrochloride salt as a yellow solid. LC-MS: [M+H⁺]=537.3. ¹H NMR (400 MHZ, CD₃OD): δ ppm 8.48-8.41 (m, 1H), 7.73-7.62 (m, 2H), 7.50-7.42 (m, 1H), 7.22-7.12 (m, 1H), 7.06 (d, J=7.8 Hz, 1H), 6.94-6.86 (m, 1H), 5.13-5.01 (m, 1H), 4.02 (s, 3H), 3.88-3.79 (m, 1H), 3.69-3.57 (m, 1H), 3.50-3.37 (m, 4H), 3.23-3.18 (m, 3H), 3.01-2.96 (m, 2H), 2.87-2.81 (m, 3H), 2.05-1.87 (m, 3H), 1.70-1.56 (m, 1H).

### Synthesis of (R)—N-(6-chloro-8-methylisoquinolin-1-yl)-2-fluoro-4-(5-(methoxymethyl)-1-methyl-1H-1,2,3-triazol-4-yl)-N-(piperidin-3-yl)benzamide (Example 637)

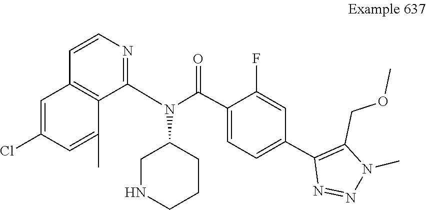 [SMILES: COCc1c(-c2ccc(C(=O)N(c3nccc4cc(Cl)cc(C)c34)[C@@H]3CCCNC3)c(F)c2)nnn1C]

Example 637 was prepared according to the procedure described for Example 816 by substituting 4-bromo-2-chloro-benzoyl chloride and 4-bromo-1-methyl-triazole with 2-fluoro-4-iodo-benzoyl chloride and 4-bromo-5-(methoxymethyl)-1-methyl-triazole, provided the title compound hydrochloride salt as an off-white solid. LC-MS: [M+H⁺]=523.3. ¹H NMR (400 MHZ, CD₃OD): δ ppm 8.49-8.41 (m, 1H), 7.72-7.63 (m, 2H), 7.50-7.43 (m, 1H), 7.16-7.01 (m, 2H), 6.97-6.88 (m, 1H), 5.15-5.01 (m, 1H), 4.50-4.40 (m, 2H), 4.06 (s, 3H), 4.02-3.96 (m, 1H), 3.89-3.79 (m, 1H), 3.71-3.58 (m, 1H), 3.46-3.37 (m, 3H), 3.09-2.95 (m, 1H), 2.89-2.81 (m, 3H), 2.23-1.87 (m, 3H), 1.73-1.26 (m, 1H).

### Synthesis of (R)-6-(1-(2-methoxyethyl)-1H-1,2,3-triazol-4-yl)-N-(3-methylthieno[3,2-c]pyridin-4-yl)-N-(piperidin-3-yl)nicotinamide (Example 638)

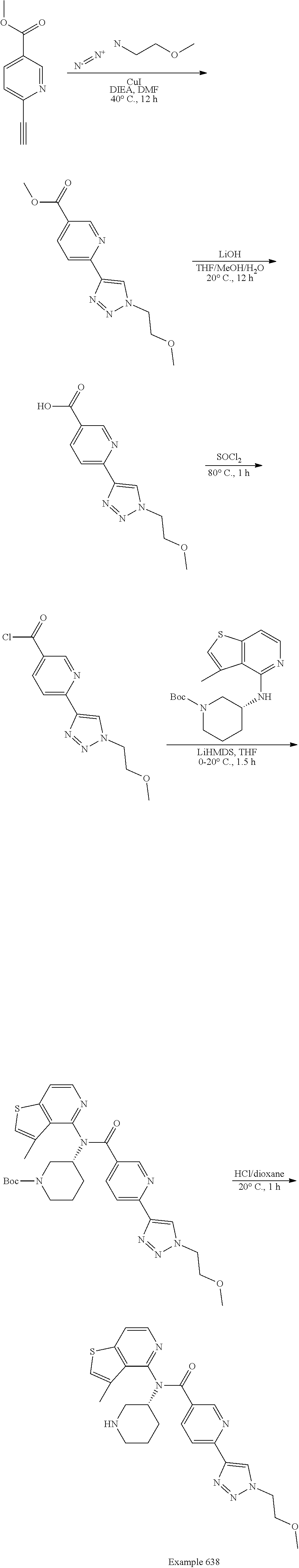

### Step 1:

To a solution of methyl 6-ethynylpyridine-3-carboxylate (0.4 g, 2.48 mmol) in DMF (4 mL) were added CuI (9.45 mg, 49.64 μmol) and DIPEA (864.65 μL, 4.96 mmol) at 20° C. Then 1-azido-2-methoxy-ethane (250.95 mg, 2.48 mmol) was added to the above mixture dropwise at 20° C. under N₂. The resulting mixture was stirred at 40° C. for 12 h under N₂ atmosphere. The reaction mixture was diluted with water (10 mL) and extracted with EtOAc (6×10 mL), dried over Na₂SO₄, filtered and concentrated. The residue was purified by column chromatography to give methyl 6-[1-(2-methoxyethyl)triazol-4-yl]pyridine-3-carboxylate (0.3 g, 41%) as a yellow solid. LC-MS: [M+H]=263.2.

### Step 2:

To a solution of methyl 6-[1-(2-methoxyethyl)triazol-4-yl]pyridine-3-carboxylate (0.3 g, 1.03 mmol) in MeOH (2.4 mL), THF (2.4 mL) and H₂O (1.2 mL) was added LiOH (123.27 mg, 5.15 mmol) at 20° C. Then the mixture was stirred at 20° C. for 12 h before it was acidified with 1N aq. HCl to pH 2. The reaction mixture was extracted with EtOAc (50 mL), dried over Na₂SO₄, filtered, and concentrated under reduced pressure to give 6-[1-(2-methoxyethyl)triazol-4-yl]pyridine-3-carboxylic acid (0.30 g, crude) as a yellow solid. LC-MS: [M+H]=249.1.

### Step 3:

A solution of 6-[1-(2-methoxyethyl)triazol-4-yl]pyridine-3-carboxylic acid (0.25 g, 1.01 mmol) in SOCl₂ (1.5 mL) was stirred at 80° C. for 1 h under N₂ atmosphere. The reaction mixture was concentrated under reduced pressure to crude 6-[1-(2-methoxyethyl)triazol-4-yl]pyridine-3-carbonyl chloride (0.25 g) as a yellow solid. LC-MS: [M+H]=263.1.

### Steps 4-5:

Example 638 was prepared according to the procedure described for Example 444 by substituting tert-butyl (3R)-3-[(6-chloro-8-methyl-1-isoquinolyl)amino]piperidine-1-carboxylate and 2-fluoro-4-(1-methyltriazol-4-yl)benzoyl chloride with tert-butyl (3R)-3-[(3-methylthieno[3,2-c]pyridin-4-yl)amino]piperidine-1-carboxylate and 6-[1-(2-methoxyethyl)triazol-4-yl]pyridine-3-carbonyl chloride, provided the title compound hydrochloride salt as an off-white solid. LC-MS: [M+H]=478.3. ¹H NMR (400 MHZ, DMSO-d₆): δ ppm 9.21-8.90 (m, 2H), 8.48 (t, J=2.7 Hz, 2H), 8.19 (d, J=2.1 Hz, 1H), 8.14-8.10 (m, 1H), 7.74 (d, J=8.3 Hz, 1H), 7.61 (dd, J=2.3, 8.3 Hz, 1H), 7.52 (d, J=1.1 Hz, 1H), 5.10-4.97 (m, 1H), 4.53 (t, J=5.1 Hz, 2H), 3.76 (br s, 1H), 3.72 (t, J=5.2 Hz, 2H), 3.25 (br d, J=13.3 Hz, 1H), 3.21 (s, 3H), 2.82-2.71 (m, 1H), 2.42 (d, J=0.9 Hz, 3H), 2.40-2.31 (m, 1H), 1.85-1.64 (m, 3H), 1.15-1.04 (m, 1H).

### Synthesis of (R)-4-(1-(2-methoxyethyl)-1H-1,2,3-triazol-4-yl)-N-(3-methylthieno[3,2-c]pyridin-4-yl)-N-(piperidin-3-yl)benzamide (Example 639)

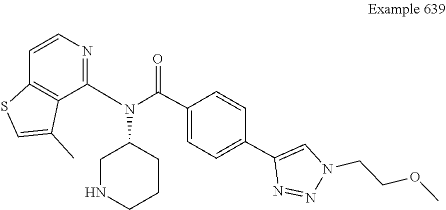 [SMILES: COCCn1cc(-c2ccc(C(=O)N(c3nccc4scc(C)c34)[C@@H]3CCCNC3)cc2)nn1]

Example 639 was prepared according to the procedure described for Example 699 by substituting 2-fluoro-4-iodo-benzoyl chloride and 2-bromo-5-methyl-1,3,4-oxadiazole with 4-iodo-benzoyl chloride and 4-bromo-1-(2-methoxyethyl)triazole, provided the title compound hydrochloride salt as an off-white solid. LC-MS: [M+H]=477.3. ¹H NMR (400 MHZ, CD₃OD): δ ppm 8.45 (d, J=5.5 Hz, 1H), 8.23 (s, 1H), 7.96 (d, J=5.5 Hz, 1H), 7.52 (br d, J=8.4 Hz, 2H), 7.31 (s, 1H), 7.23 (br d, J=8.5 Hz, 2H), 5.22-5.10 (m, 1H), 4.56 (t, J=5.0 Hz, 2H), 3.96-3.87 (m, 1H), 3.79-3.75 (m, 2H), 3.72 (br d, J=4.0 Hz, 1H), 3.38 (br s, 3H), 2.98-2.87 (m, 1H), 2.61-2.35 (m, 4H), 1.96-1.74 (m, 3H), 1.38-1.23 (m, 1H).

### Synthesis of (R)-2-fluoro-4-(1-(2-methoxyethyl)-1H-1,2,3-triazol-4-yl)-N-(3-methylthieno[3,2-c]pyridin-4-yl)-N-(piperidin-3-yl)benzamide (Example 640)

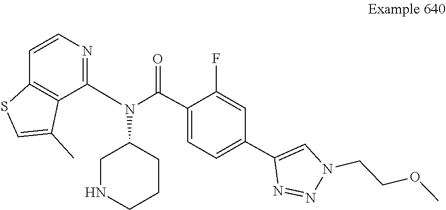 [SMILES: COCCn1cc(-c2ccc(C(=O)N(c3nccc4scc(C)c34)[C@@H]3CCCNC3)c(F)c2)nn1]

Example 640 was prepared according to the procedure described for Example 699 by substituting 2-bromo-5-methyl-1,3,4-oxadiazole with 4-bromo-1-(2-methoxyethyl) triazole, provided the title compound hydrochloride salt as an off-white solid. LC-MS: [M+H]=495.3. ¹H NMR (400 MHZ, DMSO-d₆): δ ppm 9.66-9.08 (m, 2H), 8.53 (s, 1H), 8.42-8.32 (m, 1H), 8.02 (d, J=5.4 Hz, 1H), 7.73-7.57 (m, 1H), 7.54-7.39 (m, 1H), 7.36-7.25 (m, 1H), 7.05-6.90 (m, 1H), 5.04-4.73 (m, 1H), 4.50 (br t, J=4.9 Hz, 2H), 3.87-3.62 (m, 3H), 3.46 (q, J=11.0 Hz, 1H), 3.34-3.10 (m, 5H), 2.80-2.55 (m, 3H), 1.98-1.60 (m, 3H), 1.19-0.95 (m, 1H).

### Synthesis of (R)—N-(6,8-dimethylisoquinolin-1-yl)-2-fluoro-4-(5-methyl-1,3,4-thiadiazol-2-yl)-N-(piperidin-3-yl)benzamide (Example 641)

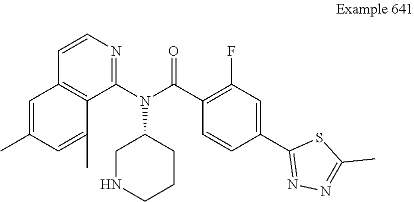

Example 641 was prepared according to the procedure described for Example 701 by substituting 5-bromopyridine-2-carbonyl chloride with 4-bromo-2-fluoro-benzoyl chloride, provided the title compound hydrochloride salt as a yellow solid. LC-MS: [M+H⁺]=476.3. ¹H NMR (400 MHZ, CD₃OD): δ ppm 8.38 (d, J=5.5 Hz, 1H), 7.68 (d, J=5.6 Hz, 1H), 7.48-7.37 (m, 3H), 7.29 (d, J=8.0 Hz, 1H), 7.04 (t, J=7.5 Hz, 1H), 5.08-4.98 (m, 1H), 4.06-3.96 (m, 1H), 3.80 (t, J=11.4 Hz, 1H), 3.40 (br d, J=12.4 Hz, 1H), 3.05-2.94 (m, 1H), 2.82 (s, 3H), 2.75 (s, 3H), 2.41-2.37 (m, 3H), 1.97-1.87 (m, 3H), 1.59-1.45 (m, 1H).

### Synthesis of (R)—N-(6,8-dimethylisoquinolin-1-yl)-6-(5-methyl-1,3,4-oxadiazol-2-yl)-N-(piperidin-3-yl)nicotinamide (Example 642)

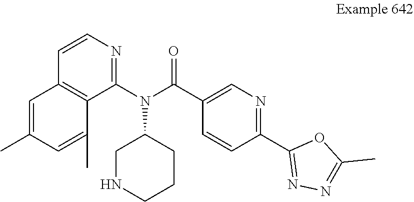

Example 642 was prepared according to the procedure described for Example 648 by substituting 2, 4-bis (4-methoxyphenyl)-2, 4-dithioxo-1, 3, 2, 4 dithiadiphosphetane with POCl₃ at step 2, provided the title compound as an off-white solid. LC-MS: [M+H]=443.3. ¹H NMR (400 MHZ, CD₃OD): δ ppm 1.17-1.33 (m, 1H) 1.60-1.79 (m, 3H) 2.36 (s, 3H) 2.45-2.58 (m, 4H) 2.63-2.86 (m, 4H) 2.99 (br d, J=12.38 Hz, 1H) 3.69 (br d, J=11.88 Hz, 1H) 4.67-4.77 (m, 1H) 7.27-7.32 (m, 1H) 7.46 (s, 1H) 7.64-7.71 (m, 2H) 7.78 (d, J=8.25 Hz, 1H) 8.33 (d, J=1.50 Hz, 1H) 8.40-8.46 (m, 1H).

### Synthesis of (R)—N-(6,8-dimethylisoquinolin-1-yl)-2-fluoro-4-(5-methyl-1,3,4-oxadiazol-2-yl)-N-(piperidin-3-yl)benzamide (Example 643)

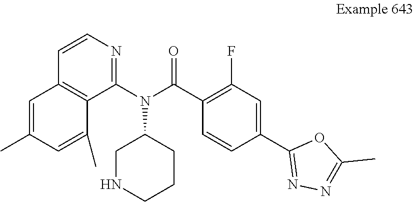

Example 643 was prepared according to the procedure described for Example 701 by substituting 5-bromopyridine-2-carbonyl chloride and 2-bromo-5-methyl-1,3,4-thiadiazole with 2-fluoro-4-iodo-benzoyl chloride and 2-bromo-5-methyl-1,3,4-oxadiazole, provided the title compound as an off-white solid. LC-MS: [M+H]=460.3. ¹H NMR (400 MHZ, CD₃OD): δ ppm 1.16-1.43 (m, 1H) 1.61-1.81 (m, 3H) 2.39 (s, 3H) 2.52 (s, 4H) 2.64-2.74 (m, 1H) 2.79-2.86 (m, 3H) 2.96-3.05 (m, 1H) 3.74 (br d, J=11.76 Hz, 1H) 4.63-4.75 (m, 1H) 7.00-7.11 (m, 1H) 7.32-7.38 (m, 2H) 7.39-7.46 (m, 2H) 7.57 (d, J=5.50 Hz, 1H) 8.28-8.36 (m, 1H).

### Synthesis of 4-(2,2-dimethylmorpholin-4-yl)-2-fluoro-N-(8-methyl-1-isoquinolyl)-N-[(3R)-3-piperidyl] benzamide (Example 644)

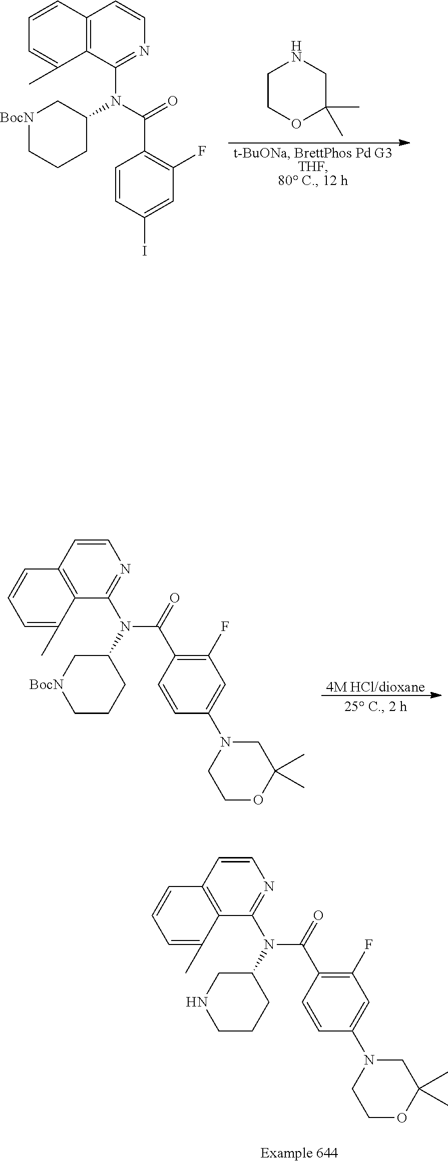

### Step 1:

A mixture of tert-butyl (3R)-3-[(2-fluoro-4-iodo-benzoyl)-(8-methyl-1-isoquinolyl) amino]piperidine-1-carboxylate (250 mg, 424.13 μmol), 2,2-dimethylmorpholine (53.73 mg, 466.55 μmol), sodium 2-methylpropan-2-olate (122.28 mg, 1.27 mmol), and [2-(2-aminophenyl) phenyl]-methylsulfonyloxy-palladium dicyclohexyl-[3,6-dimethoxy-2-(2,4,6-triisopropylphenyl) phenyl]phosphane (11.53 mg, 12.72 μmol) in THF (5 mL) was degassed with N₂ at 20° C. Then the mixture was heated to 80° C. and stirred for 12 h under N₂ atmosphere. The reaction mixture was concentrated. The residue was purified by column chromatography (SiO₂, Petroleum ether/EtOAc=8/1 to 7/1) to give tert-butyl (3R)-3-[[4-(2,2-dimethylmorpholin-4-yl)-2-fluoro-benzoyl]-(8-methyl-1-isoquinolyl)amino]piperidine-1-carboxylate (140 mg, 57%) as a white solid. LC-MS: [M+H⁺]=577.4.

### Step 2:

The title compound hydrochloride salt was obtained as an off-white solid by deprotection Method A used in Example 97. LC-MS: [M+H⁺]=477.3. ¹H NMR (400 MHZ, CD₃OD): δ ppm 8.47-8.38 (m, 1H), 7.76 (d, J=5.5 Hz, 1H), 7.70 (br d, J=8.1 Hz, 1H), 7.52 (t, J=7.7 Hz, 1H), 7.42 (br d, J=7.0 Hz, 1H), 6.58 (t, J=8.6 Hz, 1H), 6.30 (dd, J=1.9, 14.5 Hz, 1H), 6.15 (dd, J=1.9, 8.8 Hz, 1H), 5.12-4.99 (m, 1H), 4.92 (br s, 1H), 3.96-3.87 (m, 1H), 3.86-3.76 (m, 1H), 3.71-3.65 (m, 2H), 3.39 (br d, J=12.6 Hz, 1H), 3.04-2.93 (m, 3H), 2.87 (s, 2H), 2.83-2.74 (m, 3H), 2.01-1.79 (m, 3H), 1.62-1.44 (m, 1H), 1.13 (s, 6H).

### Synthesis of 4-(2,3-dihydro-1,4-benzoxazin-4-yl)-2-fluoro-N-(8-methyl-1-isoquinolyl)-N-[(3R)-3-piperidyl] benzamide (Example 645)

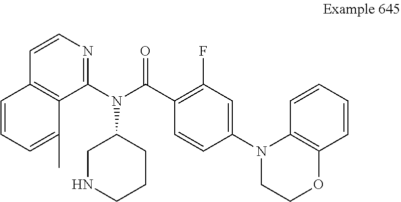 [SMILES: Cc1cccc2ccnc(N(C(=O)c3ccc(N4CCOc5ccccc54)cc3F)[C@@H]3CCCNC3)c12]

Example 645 was prepared according to the procedure described for Example 644 by substituting 2,2-dimethylmorpholine with 3,4-dihydro-2H-1,4-benzoxazine to afford the title compound as an off-white solid. LC-MS: [M+H⁺]=497.3. ¹H NMR (CD₃OD, 400 MHZ): δ ppm 8.45-8.37 (m, 1H), 7.79-7.65 (m, 2H), 7.58-7.49 (m, 1H), 7.47-7.39 (m, 1H), 6.85-6.75 (m, 2H), 6.73-6.60 (m, 2H), 6.57-6.48 (m, 2H), 6.47-6.40 (m, 1H), 5.13-5.02 (m, 1H), 4.08-4.00 (m, 2H), 3.97-3.90 (m, 1H), 3.89-3.78 (m, 1H), 3.50-3.44 (m, 2H), 3.41 (br d, J=13.3 Hz, 1H), 3.09-2.94 (m, 1H), 2.82-2.76 (m, 3H), 2.04-1.84 (m, 3H), 1.75-1.60 (m, 1H).

### Synthesis of 4-(2,3-dihydropyrido[3,2-b][1,4]oxazin-4-yl)-2-fluoro-N-(8-methyl-1-isoquinolyl)-N-[(3R)-3-piperidyl]benzamide (Example 646) (ITC-5316-001)

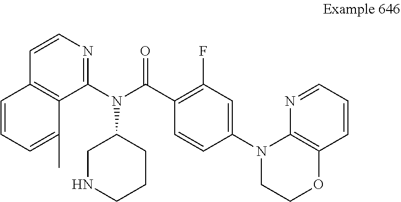 [SMILES: Cc1cccc2ccnc(N(C(=O)c3ccc(N4CCOc5cccnc54)cc3F)[C@@H]3CCCNC3)c12]

Example 646 was prepared according to the procedure described for Example 644 by substituting 2,2-dimethylmorpholine with 3,4-dihydro-2H-pyrido[3,2-b][1,4]oxazine to afford the title compound as an off-white solid. LC-MS: [M+H⁺]=498.3. ¹H NMR (CD₃OD, 400 MHZ): δ ppm 8.54-8.43 (m, 1H), 7.81-7.71 (m, 2H), 7.62-7.55 (m, 2H), 7.54-7.49 (m, 1H), 7.42 (d, J=6.3 Hz, 1H), 7.21-7.13 (m, 2H), 7.03 (dd, J=6.4, 7.9 Hz, 1H), 6.95 (dd, J=1.8, 8.4 Hz, 1H), 5.07 (br s, 1H), 4.36 (t, J=4.5 Hz, 2H), 4.01 (br dd, J=1.4, 10.7 Hz, 1H), 3.86-3.73 (m, 1H), 3.71-3.63 (m, 2H), 3.40 (br d, J=12.1 Hz, 1H), 3.02-2.94 (m, 1H), 2.92-2.84 (m, 3H), 2.09-1.79 (m, 3H), 1.46 (br d, J=8.1 Hz, 1H).

### Synthesis of N-(6-fluoro-8-methyl-1-isoquinolyl)-6-(5-methyl-1, 3, 4-thiadiazol-2-yl)-N-[(3R)-3-piperidyl] pyridine-3-carboxamide (Example 647)

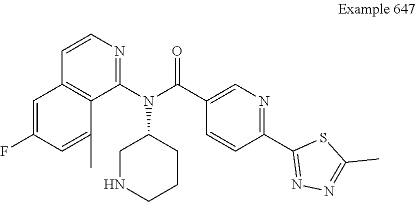 [SMILES: Cc1nnc(-c2ccc(C(=O)N(c3nccc4cc(F)cc(C)c34)[C@@H]3CCCNC3)cn2)s1]

Example 647 was prepared according to the procedure described for Example 629 by substituting tert-butyl (3R)-3-[(3-methylthieno[3,2-c]pyridin-4-yl)amino]piperidine-1-carboxylate with tert-butyl (R)-3-((6-fluoro-8-methylisoquinolin-1-yl)amino)piperidine-1-carboxylate to afford the title compound as an off-white solid. LC-MS: [M+H⁺]=463.2. ¹H NMR (CD₃OD, 400 MHZ): δ ppm 8.56-8.51 (m, 1H), 8.24-8.20 (m, 1H), 7.88-7.83 (m, 2H), 7.59-7.55 (m, 1H), 7.42 (dd, J=2.4, 8.5 Hz, 1H), 7.29 (br dd, J=2.2, 9.4 Hz, 1H), 5.14-5.05 (m, 1H), 4.03-3.92 (m, 1H), 3.86-3.76 (m, 1H), 3.43-3.37 (m, 1H), 3.05-2.94 (m, 1H), 2.85-2.75 (m, 6H), 2.01-1.82 (m, 3H), 1.35-1.24 (m, 1H).

### Synthesis of (R)—N-(6,8-dimethylisoquinolin-1-yl)-6-(5-methyl-1,3,4-thiadiazol-2-yl)-N-(piperidin-3-yl)nicotinamide (Example 648)

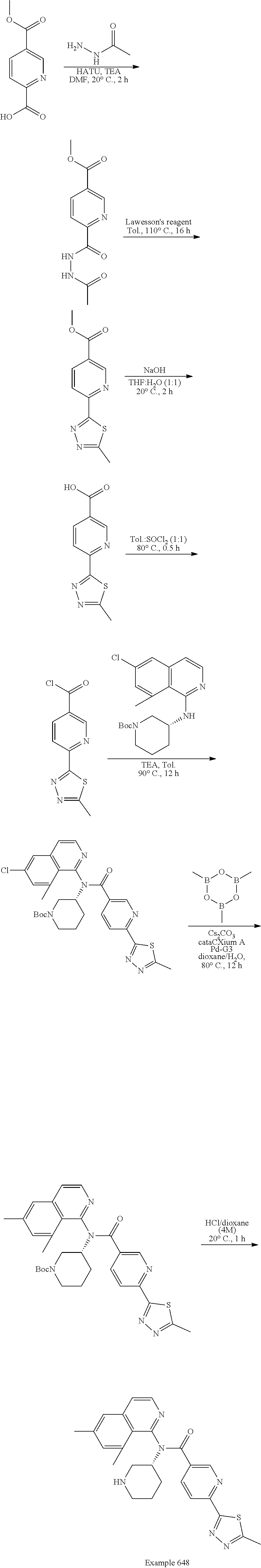

### Step 1:

A mixture of 5-methoxycarbonylpyridine-2-carboxylic acid (25 g, 132.50 mmol), acetohydrazide (20.36 g, 264.97 mmol), HATU (100.75 g, 164.97 mmol), and DIPEA (51.37 g, 397.47 mmol, 67.23 mL) in DMF (250 mL) was stirred at 20° C. for 2 h. The precipitate in the reaction mixture was collected by filtration and dried to give methyl 6-(acetamido-carbamoyl)pyridine-3-carboxylate (22 g, 68%) as a white solid.

### Step 2:

A mixture of methyl 6-(acetamidocarbamoyl) pyridine-3-carboxylate (22 g, 92.74 mmol) and 2, 4-bis (4-methoxyphenyl)-2, 4-dithioxo-1, 3, 2, 4 dithiadiphosphetane (75.02 g, 185.48 mmol) in toluene (220 mL) was degassed with N₂ at 20° C., and then the mixture was heated to 110° C. and stirred for 16 h under N₂ atmosphere. The reaction mixture was filtered and the filtrate was concentrated. The residue was stirred in EtOAc (20 mL) for 0.5 h. The precipitate was collected and dried to give methyl 6-(5-methyl-1, 3, 4-thiadiazol-2-yl) pyridine-3-carboxylate (20 g, 20%) as a white solid.

### Step 3:

A mixture of methyl 6-(5-methyl-1, 3, 4-thiadiazol-2-yl) pyridine-3-carboxylate (20 g, 85.02 mmol) and NaOH (10.20 g, 255.04 mmol) in THF (40 mL) and water (40 mL) was stirred at 20° C. for 2 h. The precipitate was collected and dried to give crude 6-(5-methyl-1, 3, 4-thiadiazol-2-yl) pyridine-3-carboxylic acid (5.4 g, 28.71%) as a white solid.

### Step 4:

A mixture of crude 6-(5-methyl-1, 3, 4-thiadiazol-2-yl) pyridine-3-carboxylic acid (5.1 g, 23.13 mmol) in SOCl₂ (30 mL) was purged. The reaction mixture was heated to 80° C. and stirred for 0.5 h under N₂ atmosphere. The reaction mixture was concentrated under reduced pressure to give crude 6-(5-methyl-1, 3, 4-thiadiazol-2-yl) pyridine-3-carbonyl chloride (5.10 g, 36%) as a yellow solid.

### Step 5:

To a solution of 6-(5-methyl-1, 3, 4-thiadiazol-2-yl) pyridine-3-carbonyl chloride (5.10 g, 12.76 mmol) in toluene (40 mL) at 0° C. under N₂ atmosphere were added TEA (4.48 mL, 31.92 mmol) and tert-butyl (3R)-3-[(6-chloro-8-methyl-1-isoquinolyl) amino] piperidine-1-carboxylate (4 g, 10.37 mmol). The mixture was heated to 90° C. and stirred for 12 h under N₂ atmosphere. The reaction mixture was concentrated under reduced pressure to give a residue, which was purified by prep-HPLC the title compound hydrochloride salt (640 mg, 10%) as a white solid.

### Step 6:

To a solution of tert-butyl (3R)-3-[(6-chloro-8-methyl-1-isoquinolyl)-[6-(5-methyl-1, 3, 4-thiadiazol-2-yl) pyridine-3-carbonyl] amino] piperidine-1-carboxylate (150 mg, 172.68 μmol) and 2, 4, 6-trimethyl-1, 3, 5, 2, 4, 6-trioxatriborinane (43.35 mg, 345.36 μmol) in dioxane (2 mL) and H₂O (0.4 mL) at 25° C. under N₂ atmosphere were added Cs₂CO₃ (168.79 mg, 518.03 μmol) and [2-(2-aminophenyl) phenyl] palladium (1+); bis(1-adamantyl)-butyl-phosphane; methanesulfonate (12.58 mg, 17.27 μmol). The mixture was heated to 80° C. and stirred for 12 h. The reaction mixture was concentrated. The residue was purified by prep-TLC (SiO₂, EtOAc:petroleum ether=3:1) to give tert-butyl (R)-3-(N-(6,8-dimethylisoquinolin-1-yl)-6-(5-methyl-1,3,4-thiadiazol-2-yl)nicotinamido) piperidine-1-carboxylate (130 mg, 89%) as a white solid.

### Step 7:

To a solution of tert-butyl (R)-3-(N-(6,8-dimethylisoquinolin-1-yl)-6-(5-methyl-1,3,4-thiadiazol-2-yl)nicotinamido) piperidine-1-carboxylate (120 mg, 214.79 μmol) in 4 M HCl in dioxane (2 mL) was stirred at 20° C. for 1 h. The reaction mixture was concentrated. The residue was purified by prep-HPLC to give the title compound hydrochloride salt (42.9 mg, 43%) as a white solid. LC-MS: [M+H⁺]=459.3. ¹H NMR (400 MHZ, CD₃OD): δ ppm 8.53-8.40 (m, 1H), 8.28 (d, J=1.4 Hz, 1H), 7.90-7.73 (m, 2H), 7.62 (dd, J=2.1, 8.3 Hz, 1H), 7.55-7.47 (m, 1H), 7.33 (s, 1H), 5.17-5.01 (m, 1H), 4.00 (br d, J=11.6 Hz, 1H), 3.86-3.74 (m, 1H), 3.53-3.37 (m, 1H), 3.08-2.92 (m, 1H), 2.82-2.70 (m, 6H), 2.37 (s, 3H), 2.22-1.73 (m, 3H), 1.55-1.37 (m, 1H).

### Synthesis of (R)-4-(5-ethyl-1,3,4-oxadiazol-2-yl)-2-fluoro-N-(3-methylthieno[3,2-c]pyridin-4-yl)-N-(piperidin-3-yl)benzamide (Example 649)

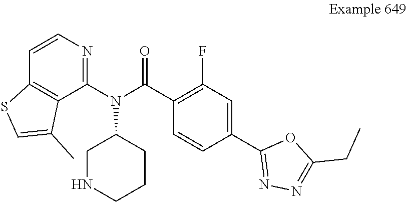 [SMILES: CCc1nnc(-c2ccc(C(=O)N(c3nccc4scc(C)c34)[C@@H]3CCCNC3)c(F)c2)o1]

Example 649 was prepared according to the procedure described for Example 699 by substituting 2-bromo-5-methyl-1,3,4-oxadiazole with 2-bromo-5-ethyl-1,3,4-oxadiazole, provided the title compound as a white solid. LC-MS: [M+H]=466.2. ¹H NMR (400 MHZ, DMSO-d₆): δ ppm 8.37-8.28 (m, 1H), 8.04-7.94 (m, 1H), 7.61-7.48 (m, 2H), 7.42 (br d, J=8.1 Hz, 1H), 7.15 (q, J=7.8 Hz, 1H), 4.57-4.36 (m, 1H), 3.04-2.95 (m, 1H), 2.89-2.80 (m, 3H), 2.62-2.52 (m, 3H), 2.35-2.20 (m, 2H), 1.67-1.43 (m, 3H), 1.25 (t, J=7.5 Hz, 3H), 0.91-0.81 (m, 1H).

### Synthesis of 2-fluoro-N-(6-fluoro-8-methyl-1-isoquinolyl)-4-(5-hydroxy-1,3,4-thiadiazol-2-yl)-N-[(3R)-3-piperidyl]benzamide (Example 650)

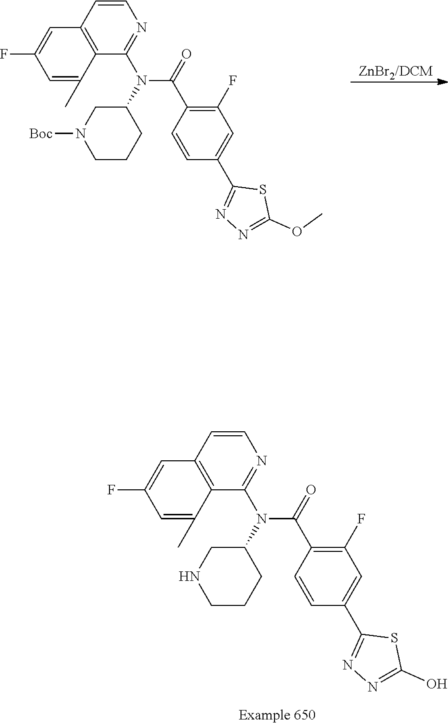 [SMILES: COc1nnc(-c2ccc(C(=O)N(c3nccc4cc(F)cc(C)c34)[C@@H]3CCCN(C(=O)OC(C)(C)C)C3)c(F)c2)s1 ; "ZnBr2/DCM" ; Cc1cc(F)cc2ccnc(N(C(=O)c3ccc(-c4nnc(O)s4)cc3F)[C@@H]3CCCNC3)c12]

Example 650 was prepared following the synthesis for example 833 by substituting 2-bromo-5-methyl-1,3,4-thiadiazole with 2-bromo-5-methoxy-1,3,4-thiadiazole to afford tert-butyl (3R)-3-[[2-fluoro-4-(5-methoxy-1,3,4-thiadiazol-2-yl)benzoyl]-(6-fluoro-8-methyl-1-isoquinolyl)amino]piperidine-1-carboxylate.

### Step 1:

To a solution of tert-butyl (3R)-3-[[2-fluoro-4-(5-methoxy-1,3,4-thiadiazol-2-yl)benzoyl]-(6-fluoro-8-methyl-1-isoquinolyl)amino]piperidine-1-carboxylate (23 mg, 38.61 μmol) in DCM (2 mL) was added ZnBr₂ (86.96 mg, 386.13 μmol) at 20° C. under N₂ atmosphere, then the mixture was stirred at 20° C. for 0.5 h. The reaction mixture was filtered and concentrated. The residue was purified by prep-HPLC to give the title compound (2.5 mg, 12%) as a white solid. LC-MS: [M+H⁺]=482.2. ¹H NMR (400 MHZ, CD₃OD): δ ppm 8.44-8.36 (m, 1H), 7.74-7.67 (m, 1H), 7.39-7.30 (m, 2H), 7.24-7.14 (m, 1H), 7.07-7.02 (m, 1H), 6.96-6.90 (m, 1H), 5.07-4.96 (m, 1H), 4.00-3.89 (m, 1H), 3.81-3.60 (m, 2H), 2.99-2.91 (m, 1H), 2.89-2.83 (m, 3H), 2.00-1.82 (m, 3H), 1.65-1.47 (m, 1H), 1.36-1.26 (m, 2H).

### Synthesis of 2-fluoro-N-(3-isopropenylthieno[3,2-c]686yridine-4-yl)-4-(5-methyl-1,3,4-thiadiazol-2-yl)-N-[(3R)-3-piperidyl]benzamide (Example 651)

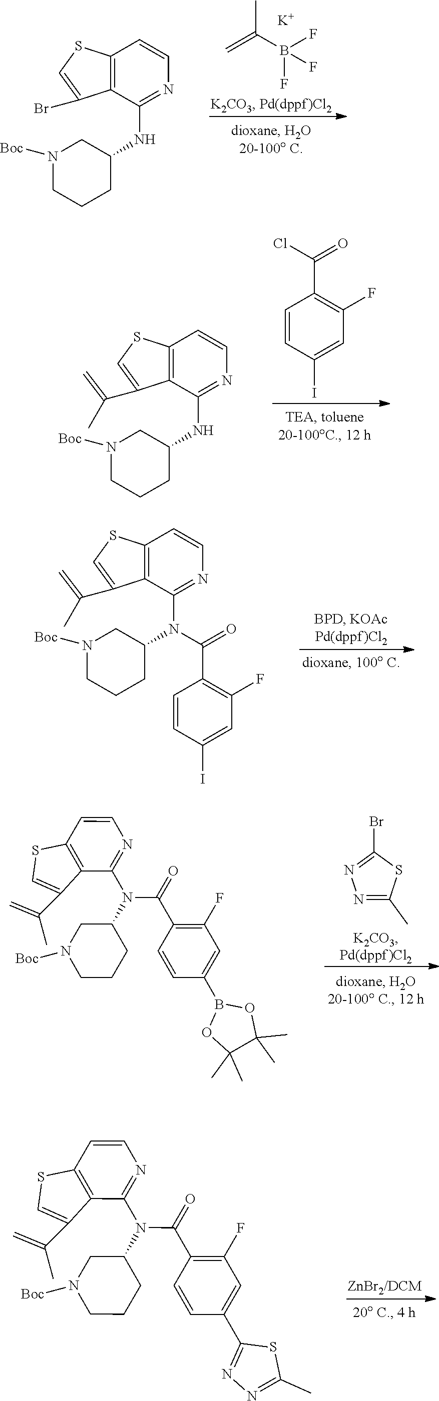

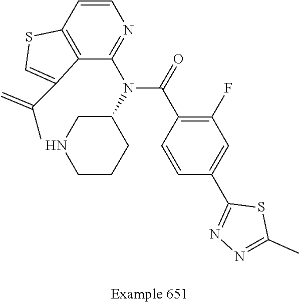 [SMILES: C=C(C)c1csc2ccnc(N(C(=O)c3ccc(-c4nnc(C)s4)cc3F)[C@@H]3CCCNC3)c12]

### Step 1:

To a solution of tert-butyl (3R)-3-[(3-bromothieno[3,2-c]687yridine-4-yl)amino]piperidine-1-carboxylate (500 mg, 1.09 mmol) in dioxane (9 mL) was added potassium isopropenyltrifluoroborate (322.98 mg, 2.18 mmol), K₂CO₃ (452.48 mg, 3.27 mmol), Pd(dppf)Cl₂ (79.85 mg, 109.13 μmol) and H₂O (1 mL) at 20° C. under N₂ atmosphere, then the mixture was stirred at 100° C. for 3 h. The reaction mixture was diluted with water (15 mL) and then extracted with EtOAc (3×20 mL). The combined organic layer was washed with brine (20 mL), dried over Na₂SO₄, filtered and concentrated. The residue was purified by flash column using 5% EtOAc in petroleum ether to give tert-butyl (3R)-3-[(3-isopropenylthieno[3,2-c]687yridine-4-yl)amino]piperidine-1-carboxylate (400 mg, 88%) was obtained as an off-white solid.

### Step 2:

To a solution of 2-fluoro-4-iodo-benzoyl chloride (533.10 mg, 1.69 mmol, 2 eq) in toluene. (6 mL) was added TEA (682.71 mg, 6.75 mmol, 939.07 μL) and tert-butyl (3R)-3-[(3-isopropenylthieno[3,2-c]687yridine-4-yl)amino]piperidine-1-carboxylate (350 mg, 843.35 μmol) at 20° C. under N₂ atmosphere, then the mixture was stirred at 100° C. for 12 h. The reaction mixture was diluted with water (5 mL), then extracted with EtOAc (3×20 mL). The combined organic layer was washed with brine (20 mL), dried over Na₂SO₄, filtered and concentrated. The residue was purified by flash column using 10% EtOAc in petroleum ether to give tert-butyl (3R)-3-[(2-fluoro-4-iodo-benzoyl)-(3-isopropenylthieno[3,2-c]687yridine-4-yl)amino]piperidine-1-carboxylate (645 mg, 86%) as an off-white solid. LC-MS: [M+H⁺]=622.2.

### Step 3:

To a solution of tert-butyl (3R)-3-[(2-fluoro-4-iodo-benzoyl)-(3-isopropenylthieno-[3,2-c]687yridine-4-yl)amino]piperidine-1-carboxylate (595 mg, 765.88 μmol) in dioxane (15 mL) was added BPD (972.44 mg, 3.83 mmol), KOAc (376 mg, 3.83 mmol) and Pd(dppf)Cl₂ (112 mg, 153.18 μmol) at 20° C. under N₂ atmosphere, then the mixture was stirred at 100° C. for 12 h. The reaction mixture was diluted with water (15 mL), then extracted with EtOAc (3×20 mL). The combined organic layer was washed with brine (20 mL), dried over Na₂SO₄, filtered and concentrated. The residue was purified by flash column using 10% EtOAc in petroleum ether to give tert-butyl (3R)-3-[[2-fluoro-4-(4,4,5,5-tetramethyl-1,3,2-dioxaborolan-2-yl)benzoyl]-(3-isopropenylthieno[3,2-c]687yridine-4-yl) amino]piperidine-1-carboxylate (570 mg, 88%) as a white solid. LC-MS: [M+H⁺]=622.3.

### Step 4:

To a solution of tert-butyl (3R)-3-[[2-fluoro-4-(4,4,5,5-tetramethyl-1,3,2-dioxaborolan-2-yl)benzoyl]-(3-isopropenylthieno[3,2-c] 688yridine-4-yl)amino]piperidine-1-carboxylate (520.00 mg, 669.27 μmol) in dioxane (10 mL) was added 2-bromo-5-methyl-1,3,4-thiadiazole (239.65 mg, 1.34 mmol), K₂CO₃ (277.49 mg, 2.01 mmol), Pd(dppf)Cl₂ (97.94 mg, 133.85 μmol) and H₂O (2 mL) at 20° C. under N₂ atmosphere, then the mixture was stirred at 100° C. for 12 h. The reaction mixture was diluted with water (10 mL), then extracted with EtOAc (3×15 mL). The combined organic layer was washed with brine (3×15 mL), dried over Na₂SO₄, filtered and concentrated. The residue was purified by flash column using 5% EtOAc in petroleum ether to give tert-butyl (3R)-3-[[2-fluoro-4-(5-methyl-1,3,4-thiadiazol-2-yl)benzoyl]-(3-isopropenylthieno[3,2-c]pyridine-4-yl)amino]piperidine-1-carboxylate (460 mg, 84%) as a brown solid.

### Step 5:

To a solution of tert-butyl (3R)-3-[[2-fluoro-4-(5-methyl-1,3,4-thiadiazol-2-yl)benzoyl]-(3-isopropenylthieno[3,2-c]pyridine-4-yl)amino]piperidine-1-carboxylate (320 mg, 538.96 μmol) in dioxane (5 mL) was added ZnBr₂ (606.87 mg, 2.69 mmol) at 20° C. under N₂ atmosphere, then the mixture was stirred at 40° C. for 4 h. The reaction mixture was filtered and the filtrate was concentrated. The residue was purified by reverse-phase prep-HPLC and the pure fraction was lyophilized to give the title compound (53.7 mg, 12%) as a white solid. LC-MS: [M+H⁺]=494.1. ¹H NMR (400 MHz, DMSO-d₆): δ ppm 8.49-8.31 (m, 1H), 8.27-8.12 (m, 1H), 8.02-7.96 (m, 1H), 7.81-7.68 (m, 1H), 7.55-7.38 (m, 1H), 7.17-6.83 (m, 1H), 5.54-5.38 (m, 1H), 5.18-4.97 (m, 1H), 4.42-3.69 (m, 1H), 3.45 (br d, J=11.8 Hz, 1H), 3.18 (dt, J=5.4, 11.1 Hz, 1H), 2.83 (s, 2H), 2.73 (s, 1H), 2.59 (s, 1H), 2.36 (br t, J=11.2 Hz, 1H), 2.22 (s, 1H), 2.19-1.99 (m, 3H), 1.87-1.67 (m, 1H), 1.66-1.51 (m, 1H), 1.50-1.34 (m, 1H), 1.30-1.13 (m, 1H), 1.05-0.77 (m, 1H).

### Synthesis of N-(3-ethynylthieno[3, 2-c]pyridine-4-yl)-2-fluoro-4-(5-methyl-1, 3, 4-thiadiazol-2-yl)-N-[(3R)-3-piperidyl] benzamide (Example 652)

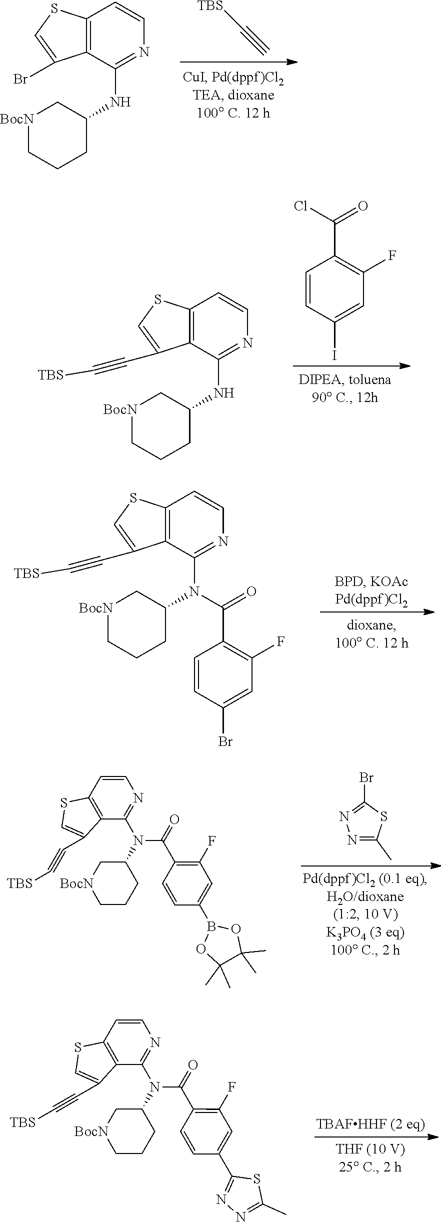

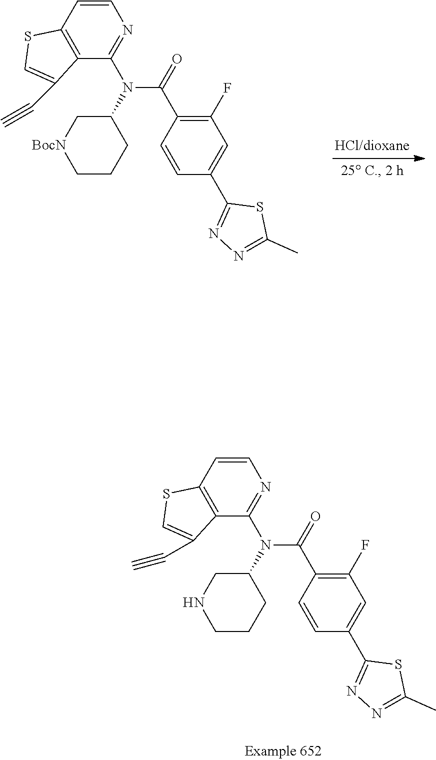

### Step 1:

To a solution of tert-butyl-ethynyl-dimethyl-silane (340.24 mg, 2.43 mmol) in TEA (2.5 mL) and dioxane (2.5 mL) was added tert-butyl (3R)-3-[(3-bromothieno[3,2-c]pyridine-4-yl)amino]piperidine-1-carboxylate (0.5 g, 1.21 mmol), Pd(dppf)Cl₂ (88.72 mg, 121.26 μmol) and CuI (23.09 mg, 121.26 μmol). The mixture was stirred at 100° C. for 12 h. The reaction mixture was filtered and the filtrate was concentrated. The residue was purified by flash column using 5% EtOAc in petroleum ether to give tert-butyl (3R)-3-[[3-[2-[tert-butyl(dimethyl)silyl]ethynyl]thieno[3,2-c]pyridine-4-yl]amino]piperidine-1-carboxylate (0.55 g, 96%) as a white solid.

### Step 2:

To a solution of 4-bromo-2-fluoro-benzoyl chloride (755.05 mg, 3.18 mmol) in toluene (10 mL) was added DIPEA (410.96 mg, 3.18 mmol), tert-butyl (3R)-3-[[3-[2-[tert-butyl(dimethyl)silyl]ethynyl]thieno[3,2-c]689yridine-4-yl]amino]piperidine-1-carboxylate (0.5 g, 1.06 mmol) at 0° C. The mixture was stirred at 25° C. for 2 h. The reaction mixture was filtered and concentrated. The residue was purified by flash column using 10% EtOAc in petroleum ether to give tert-butyl (3R)-3-[(4-bromo-2-fluoro-benzoyl)-[3-[2-[tert-butyl(dimethyl)silyl]ethynyl]thieno[3,2-c]pyridine-4-yl]amino]piperidine-1-carboxylate (0.45 g, 63%) as a white solid.

### Step 3:

A mixture of tert-butyl (3R)-3-[(4-bromo-2-fluoro-benzoyl)-[3-[2-[tert-butyl(dimethyl)silyl]ethynyl]thieno[3,2-c]pyridine-4-yl]amino]piperidine-1-carboxylate (0.45 g, 668.92 μmol), BPD (509.60 mg, 2.01 mmol), Pd(dppf)Cl₂ (48.95 mg, 66.89 μmol) and KOAc (196.94 mg, 2.01 mmol) in dioxane (5 mL) was degassed with N₂, and then the mixture was stirred at 100° C. for 12 h under N₂ atmosphere. The reaction mixture was filtered and the filtrate was concentrated. The residue was purified by flash column using 10% EtOAc in petroleum ether to give tert-butyl (3R)-3-[[3-[2-[tert-butyl(dimethyl)silyl]-ethynyl]thieno[3,2-c]689yridine-4-yl]-[2-fluoro-4-(4,4,5,5-tetramethyl-1,3,2-dioxaborolan-2-yl)benzoyl]amino]piperidine-1-carboxylate (0.47 g, 93%) as a white solid.

### Step 4:

A mixture of tert-butyl (3R)-3-[3-[2-[tert-butyl(dimethyl)silyl]ethynyl]thieno[3,2-c]pyridine-4-yl]-[2-fluoro-4-(4,4,5,5-tetramethyl-1,3,2-dioxaborolan-2-yl)benzoyl]amino]-piperidine-1-carboxylate (450 mg, 625.19 μmol), 2-bromo-5-methyl-1,3,4-thiadiazole (223.86 mg, 1.25 mmol), Pd(dppf)Cl₂ (45.74 mg, 62.52 μmol), K₃PO₄ (398.12 mg, 1.88 mmol) in dioxane (5 mL) and water (1 mL) was degassed with N₂, and then the mixture was stirred at 100° C. for 2 h under N₂ atmosphere. The reaction mixture was filtered and the filtrate was concentrated. The residue was purified by flash column using 10% EtOAc in petroleum ether to give tert-butyl (3R)-3-[[3-[2-[tert-butyl(dimethyl)silyl]ethynyl]thieno[3,2-c]pyridine-4-yl]-[2-fluoro-4-(5-methyl-1,3,4-thiadiazol-2-yl) benzoyl] amino] piperidine-1-carboxylate (0.18 g, 37%) as a white solid.

### Step 5:

A mixture of tert-butyl (3R)-3-[3-[2-[tert-butyl(dimethyl)silyl]ethynyl]thieno[3,2-c]pyridine-4-yl]-[2-fluoro-4-(5-methyl-1,3,4-thiadiazol-2-yl)benzoyl]amino]piperidine-1-carboxylate (0.18 g, 260.13 μmol), TBAF (1.80 mL) in dichloromethane (2 mL), and then the mixture was stirred at 25° C. for 2 h. The reaction mixture was quenched by adding water and extracted with EtOAc. The combined organic layer was washed with brine, dried over Na₂SO₄, filtered and concentrated. The crude product (0.15 g, 89%) was obtained as a white solid and used in the next step without further purification.

### Step 6:

A mixture of tert-butyl (3R)-3-[(3-ethynylthieno[3, 2-c]pyridine-4-yl)-[2-fluoro-4-(5-methyl-1, 3, 4-thiadiazol-2-yl) benzoyl] amino] piperidine-1-carboxylate (150 mg, 233.17 μmol) in 4M HCl in dioxane (2.25 mL), and then the mixture was stirred at 25° C. for 0.5 h. The reaction mixture was filtered and concentrated. The residue was purified by reverse-phase prep-HPLC to give the title compound (95.6 mg, 81%) as a white solid. LC-MS: [M+H⁺]=478.2. ¹H NMR (400 MHZ, CD₃OD): δ=8.42 (d, J=5.5 Hz, 1H), 8.12 (s, 1H), 7.97 (d, J=5.5 Hz, 1H), 7.56-7.37 (m, 3H), 5.12-5.00 (m, 1H), 4.24-4.17 (m, 1H), 4.00-3.89 (m, 1H), 3.63 (t, J=11.8 Hz, 1H), 3.35 (br d, J=12.9 Hz, 1H), 2.97-2.82 (m, 1H), 2.76 (s, 3H), 2.14-2.01 (m, 1H), 1.97-1.78 (m, 2H), 1.18 (br dd, J=4.9, 12.1 Hz, 1H).

### Synthesis of N-(3-chlorothieno[3, 2-c] 690yridine-4-yl)-2-fluoro-4-(5-methyl-1, 3, 4-thiadiazol-2-yl)-N-[(3R)-3-piperidyl] benzamide (Example 653)

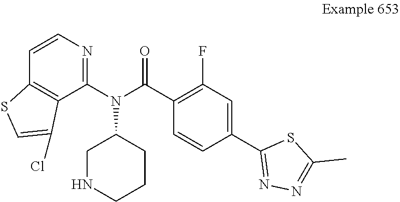 [SMILES: Cc1nnc(-c2ccc(C(=O)N(c3nccc4scc(Cl)c34)[C@@H]3CCCNC3)c(F)c2)s1]

Example 653 was prepared according to the procedure described for Example 127 by substituting tert-butyl (R)-3-((8-methylisoquinolin-1-yl)amino)piperidine-1-carboxylate with tert-butyl (R)-3-((3-chlorothieno[3,2-c]pyridine-4-yl)amino)piperidine-1-carboxylate, provided the title compound as a white solid. LC-MS: [M+H⁺]=488.1. ¹H NMR (400 MHZ, CD₃OD): δ=8.46 (d, J=5.5 Hz, 1H), 7.99 (d, J=5.5 Hz, 1H), 7.85-7.81 (m, 1H), 7.48 (d, J=10.4 Hz, 1H), 7.45-7.40 (m, 1H), 7.37-7.30 (m, 1H), 5.11 (tt, J=3.8, 11.8 Hz, 1H), 3.95 (br dd, J=3.2, 12.2 Hz, 1H), 3.67 (t, J=11.6 Hz, 1H), 3.37 (br d, J=12.6 Hz, 1H), 2.95-2.86 (m, 1H), 2.77 (s, 3H), 2.05-1.86 (m, 3H), 1.26 (dq, J=5.2, 12.0 Hz, 1H).

### Synthesis of (R)—N-(6-chloro-8-methylisoquinolin-1-yl)-4-(5-ethyl-1,3,4-oxadiazol-2-yl)-2-fluoro-N-(piperidin-3-yl)benzamide (Example 654)

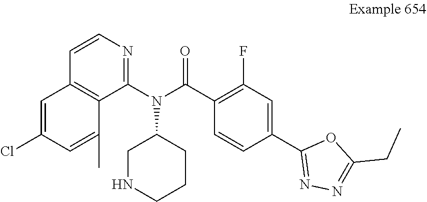 [SMILES: CCc1nnc(-c2ccc(C(=O)N(c3nccc4cc(Cl)cc(C)c34)[C@@H]3CCCNC3)c(F)c2)o1]

Example 654 was prepared according to the procedure described for Example 816 by substituting 4-bromo-2-chloro-benzoyl chloride and 4-bromo-1-methyl-triazole with 2-fluoro-4-iodo-benzoyl chloride and 2-bromo-5-ethyl-1,3,4-oxadiazole, provided the title compound as a white solid. LC-MS: [M+H]=594.3. ¹H NMR (400 MHZ, DMSO-d₆): δ ppm 8.51-8.40 (m, 1H), 7.91-7.85 (m, 1H), 7.73 (br d, J=5.5 Hz, 1H), 7.58 (br s, 1H), 7.47 (br d, J=10.0 Hz, 1H), 7.40-7.31 (m, 1H), 7.08-6.96 (m, 1H), 4.54-4.37 (m, 1H), 3.58-3.48 (m, 1H), 3.08-2.95 (m, 2H), 2.87-2.77 (m, 5H), 2.36-2.29 (m, 1H), 1.66-1.44 (m, 3H), 1.25 (br t, J=7.6 Hz, 3H), 1.17-1.05 (m, 1H).

### Synthesis of (R)-2-fluoro-N-(5-fluoro-8-methylisoquinolin-1-yl)-4-(4-methyl-1H-1,2,3-triazol-1-yl)-N-(piperidin-3-yl)benzamide (Example 655)

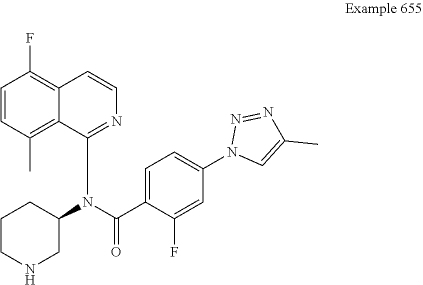 [SMILES: Cc1cn(-c2ccc(C(=O)N(c3nccc4c(F)ccc(C)c34)[C@@H]3CCCNC3)c(F)c2)nn1]

Example 655 was prepared according to the procedure described for Example 656 by substituting 4-(1-methyl-1H-1,2,3-triazol-4-yl)benzoyl with 2-fluoro-4-(1-methyl-1H-1,2,3-triazol-4-yl)benzoyl, provided the title compound hydrochloride salt as an off-white solid. LC-MS: [M+H⁺]=463.7. ¹H NMR (CD₃OD, 400 MHZ): δ ppm 8.55-8.51 (m, 2H), 8.16 (s, 1H), 7.94-7.90 (m, 1H), 7.47-7.38 (m, 2H), 7.30-7.24 (m, 2H), 7.05-7.01 (m, 1H), 5.10-5.04 (m, 1H), 4.01-3.99 (m, 1H), 3.84-3.78 (m, 1H), 3.44-3.39 (m, 1H), 3.03-2.99 (m, 1H), 2.81 (s, 3H), 2.72-2.69 (m, 1H), 2.32 (s, 3H), 1.97-1.95 (m, 2H), 1.61-1.54 (m, 1H).

### Synthesis of (R)—N-(5-fluoro-8-methylisoquinolin-1-yl)-4-(1-methyl-1H-1,2,3-triazol-4-yl)-N-(piperidin-3-yl)benzamide (Example 656)

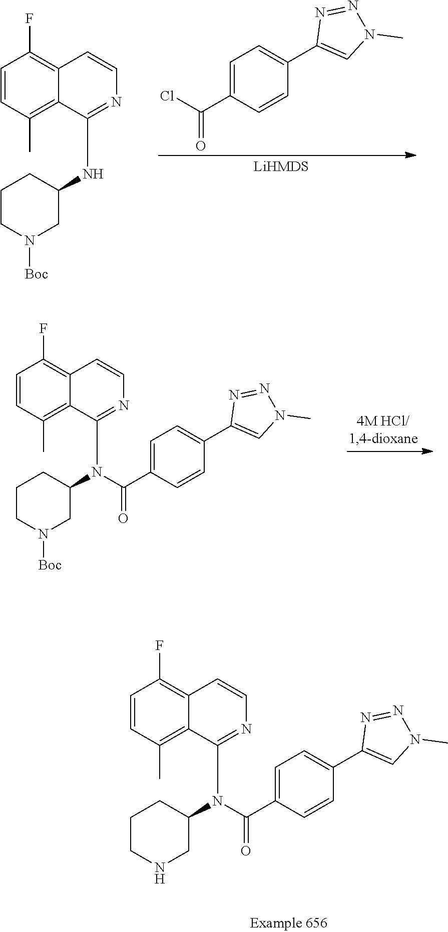 [SMILES: Cc1ccc(F)c2ccnc(N[C@@H]3CCCN(C(=O)OC(C)(C)C)C3)c12 ; Cn1cc(-c2ccc(C(=O)Cl)cc2)nn1 ; Cc1ccc(F)c2ccnc(N(C(=O)c3ccc(-c4cn(C)nn4)cc3)[C@@H]3CCCN(C(=O)OC(C)(C)C)C3)c12 ; Cc1ccc(F)c2ccnc(N(C(=O)c3ccc(-c4cn(C)nn4)cc3)[C@@H]3CCCNC3)c12]

### Step 1:

To a stirred solution of tert-butyl (R)-3-((5-fluoro-8-methylisoquinolin-1-yl)amino)-piperidine-1-carboxylate (150 mg, 0.417 mmol) in THF (10 mL) at 0° C. was added LiHMDS in THF (0.894 mL, 1.252 mmol) and stirred for 15 min and followed by addition of 4-(1-methyl-1H-1,2,3-triazol-4-yl)benzoylchloride (139 mg, 0.626 mmol) in THF (10 mL). The reaction mixture was stirred at room temperature for 15 min before it was quenched with saturated NH₄Cl (20 mL) and extracted with EtOAc (2×100 mL). The combined organic layer was washed with brine (50 mL), dried over Na₂SO₄, and concentrated. The obtained compound purified by Combi flash using 60-70% EtOAc in hexane first and followed by further purification by Prep-HPLC to provide the title compound (35 mg) as an off-white solid. LC-MS: [M+H⁺]=545.

### Step 2:

The title compound hydrochloride salt was obtained as an off-white solid by deprotection Method A used in Example 97. LC-MS: [M+H⁺]=445.6. ¹H NMR (CD₃OD, 400 MHZ): δ ppm 8.61-8.56 (m, 1H), 8.15 (s, 1H), 7.99-7.94 (m, 1H), 7.44-7.41 (m, 2H), 7.34-7.31 (m, 1H), 7.23-7.19 (m, 1H), 7.07-7.04 (m, 2H), 5.15-5.14 (m, 1H), 4.07 (s, 1H), 3.96-3.94 (m, 1H), 3.84-3.79 (m, 1H), 3.48-3.38 (m, 2H), 3.03-2.98 (m, 1H), 2.70 (s, 3H), 2.72-2.73 (m, 1H), 1.96-1.83 (m, 3H), 1.53-1.49 (m, 1H).

### Synthesis of (R)-4-(5-ethyl-1,3,4-thiadiazol-2-yl)-2-fluoro-N-(5-fluoro-8-methylisoquinolin-1-yl)-N-(piperidin-3-yl)benzamide (Example 657)

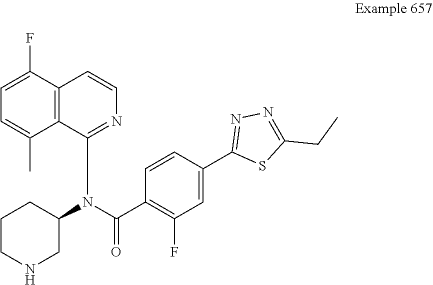 [SMILES: CCc1nnc(-c2ccc(C(=O)N(c3nccc4c(F)ccc(C)c34)[C@@H]3CCCNC3)c(F)c2)s1]

Example 657 was made following the procedure for example 761 by substituting 4-bromo-1-methyl-1H-1,2,3-triazole with 2-bromo-5-ethyl-1,3,4-thiadiazole to yield (R)-4-(5-ethyl-1,3,4-thiadiazol-2-yl)-2-fluoro-N-(5-fluoro-8-methylisoquinolin-1-yl)-N-(piperidin-3-yl)benzamide hydrochloride salt (45 mg, 95%) as light brown solid. LC-MS: [M+H⁺]=494.1. ¹H NMR (CD₃OD, 400 MHZ): δ ppm 8.54-8.51 (m, 1H), 7.93-7.89 (m, 1H), 7.47-7.38 (m, 2H), 7.30-7.24 (m, 2H), 7.00-6.95 (m, 1H), 5.09-5.02 (m, 1H), 4.02-3.99 (m, 1H), 3.84-3.78 (m, 1H), 3.45-3.34 (m, 1H), 3.14-3.09 (m, 2H), 3.02-2.95 (m, 1H), 2.81 (s, 3H), 2.77-2.71 (m, 1H), 1.98-1.91 (m, 2H), 1.59-1.55 (m, 1H), 1.40-1.36 (m, 3H).

### Synthesis of (R)—N-(6-fluoro-8-methylisoquinolin-1-yl)-4-(3-methylisoxazol-5-yl)-N-(piperidin-3-yl)piperidine-1-carboxamide (Example 658)

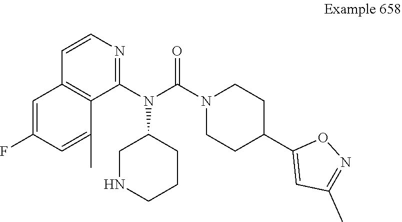 [SMILES: Cc1cc(C2CCN(C(=O)N(c3nccc4cc(F)cc(C)c34)[C@@H]3CCCNC3)CC2)on1]

Example 658 was prepared according to the procedure described for Example 243 by substituting tert-butyl (R)-3-((8-methylisoquinolin-1-yl)amino)piperidine-1-carboxylate with tert-butyl (R)-3-((6-fluoro-8-methylisoquinolin-1-yl)amino)piperidine-1-carboxylate to afford the title compound as an off-white solid. LC-MS: [M+H⁺]=452.7. ¹H NMR (CD₃OD, 400 MHz): δ ppm 8.39-8.29 (m, 1H), 7.79 (d, J=5.2 Hz, 1H), 7.59-7.55 (m, 1H), 7.47-7.38 (m, 1H), 5.85-5.73 (m, 1H), 4.83-4.80 (m, 1H), 3.93-3.90 (m, 1H), 3.73-3.62 (m, 1H), 3.52-3.49 (m, 1H), 3.34-3.30 (m, 1H), 3.12-3.11 (m, 1H), 2.94-2.91 (m, 2H), 2.73-2.67 (m, 3H), 2.57-2.50 (m, 1H), 2.18-2.17 (m, 3H), 1.91-1.83 (m, 3H), 1.69-1.59 (m, 2H), 1.39-1.33 (m, 1H), 1.08-1.01 (m, 2H), 0.39-0.33 (m, 1H).

### Synthesis of (R)—N-(5-fluoro-8-methylisoquinolin-1-yl)-4-(3-methylisoxazol-5-yl)-N-(piperidin-3-yl)piperidine-1-carboxamide (Example 659)

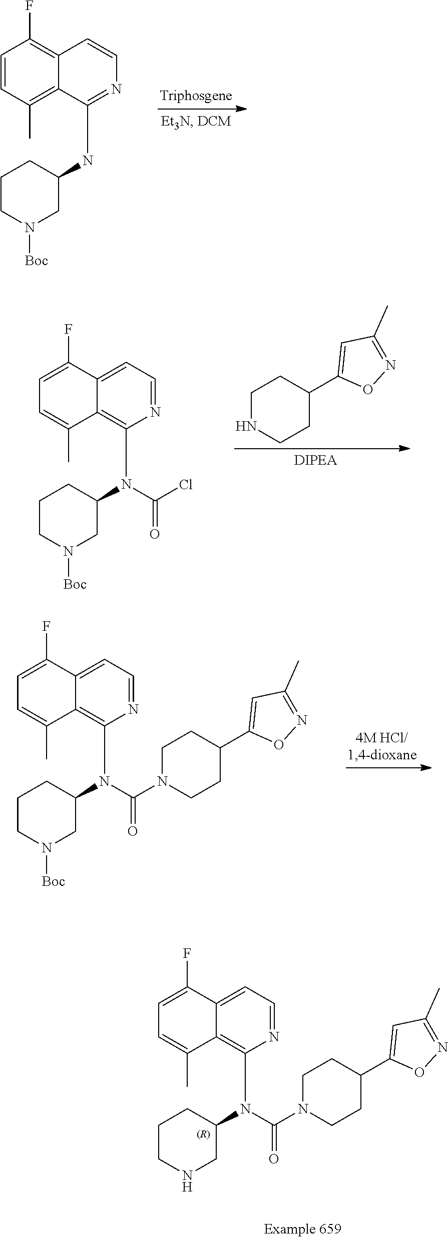

Example 659 was prepared according to the procedure described for Example 243 by substituting 3-methyl-5-(4-piperidyl) isoxazole and tert-butyl (3R)-3-[chlorocarbonyl-(8-methyl-1-isoquinolyl)amino]piperidine-1-carboxylate with 3-methyl-5-(piperidin-4-yl)isoxazole and tert-butyl (R)-3-((chlorocarbonyl)(5-fluoro-8-methylisoquinolin-1-yl)amino)piperidine-1-carboxylate, provided the title compound as a white solid. LC-MS: [M+H⁺]=452.2. ¹H NMR (CD₃OD, 400 MHZ): δ 8.49-8.39 (m, 1H), 7.94 (d, J=5.6 Hz, 1H), 7.56-7.39 (m, 2H), 5.84-5.71 (m, 1H), 4.11-4.10 (m, 1H), 3.91-3.88 (m, 1H), 3.65-3.57 (m, 2H), 3.46-3.36 (m, 1H), 3.13-3.10 (m, 1H), 2.94-2.91 (m, 1H), 2.67-2.64 (m, 1H), 2.51-2.48 (m, 1H), 2.17 (s, 3H), 2.00-1.98 (m, 1H), 1.87-1.85 (m, 3H), 1.64-1.58 (m, 2H), 1.32-1.28 (m, 2H), 1.06-0.89 (m, 3H), 0.39-0.32 (m, 1H).

### Synthesis of (R)-2-fluoro-N-(5-fluoro-8-methylisoquinolin-1-yl)-4-(5-methyl-1,3,4-oxadiazol-2-yl)-N-(piperidin-3-yl)benzamide (Example 660)

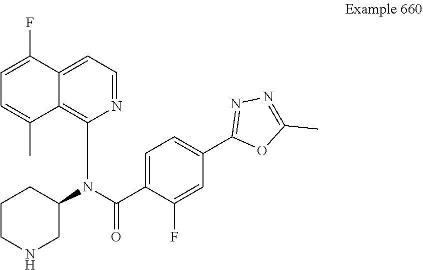 [SMILES: Cc1nnc(-c2ccc(C(=O)N(c3nccc4c(F)ccc(C)c34)[C@@H]3CCCNC3)c(F)c2)o1]

Example 660 was made following the procedure for example 761 by substituting 4-bromo-1-methyl-1H-1,2,3-triazole with 2-bromo-5-methyl-1,3,4-oxadiazole to afford the title compound formate salt as an off-white solid. LC-MS: [M+H⁺]=464.1. ¹H NMR (CD₃OD, 400 MHZ): δ ppm 8.54-8.48 (m, 1H), 7.92-7.87 (m, 1H), 7.47-7.42 (m, 2H), 7.38-7.36 (m, 1H), 7.30-7.23 (m, 1H), 7.05-7.01 (m, 1H), 5.07-5.00 (m, 1H), 4.00-3.97 (m, 1H), 3.80-3.74 (m, 1H), 3.38-3.35 (m, 1H), 2.96-2.95 (m, 1H), 2.81 (s, 3H), 2.67-2.65 (m, 1H), 2.52 (s, 3H), 1.96-1.90 (m, 2H), 1.60-1.54 (m, 1H).

### Synthesis of (R)-2-fluoro-N-(5-fluoro-8-methylisoquinolin-1-yl)-N-(piperidin-3-yl)-4-(pyrimidin-2-ylamino)benzamide (Example 661)

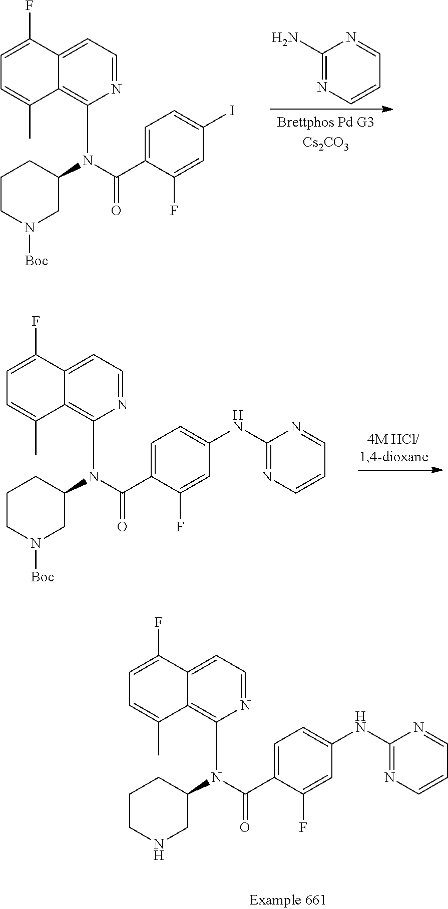 [SMILES: Cc1ccc(F)c2ccnc(N(C(=O)c3ccc(I)cc3F)[C@@H]3CCCN(C(=O)OC(C)(C)C)C3)c12 ; Nc1ncccn1 ; Cc1ccc(F)c2ccnc(N(C(=O)c3ccc(Nc4ncccn4)cc3F)[C@@H]3CCCN(C(=O)OC(C)(C)C)C3)c12 ; Cc1ccc(F)c2ccnc(N(C(=O)c3ccc(Nc4ncccn4)cc3F)[C@@H]3CCCNC3)c12]

### Step 1:

To a stirred suspension of tert-butyl (R)-3-(2-fluoro-N-(5-fluoro-8-methylisoquinolin-1-yl)-4-iodobenzamido)piperidine-1-carboxylate (150 mg, 0.247 mmol) and pyrimidin-2-amine (35 mg, 0.370 mmol) in 1,4-Dioxane (3 mL) was added Cs₂CO₃ (241 mg, 0.741 mmol) and the reaction mixture was degassed with N₂ for 10 min and followed by addition of Brettphos Pd (II) G3 (22 mg, 0.025 mmol) was added to reaction mixture and stirred at 110° C. for 16 h. The reaction mixture was filtered and the filtrate was concentrated. The residue was purified by Combi-flash using 35-40% EtOAc in hexane to afford tert-butyl (R)-3-(2-fluoro-N-(5-fluoro-8-methylisoquinolin-1-yl)-4-(pyrimidin-2-ylamino)benzamido)piperidine-1-carboxylate (80 mg, 54%) as an off-white solid.

### Step 2:

The title compound hydrochloride salt was obtained as an off-white solid by deprotection Method A used in Example 97. LC-MS: [M+H⁺]=475.65. ¹H NMR (CD₃OD, 400 MHZ): δ ppm 8.61-8.49 (m, 3H), 7.93-7.88 (m, 1H), 7.48-7.37 (m, 2H), 7.27-7.21 (m, 1H), 6.96-6.94 (m, 1H), 6.87 (d, J=8.4 Hz, 1H), 6.71-6.66 (m, 1H), 5.10-5.03 (m, 1H), 3.96-3.91 (m, 1H), 3.84-3.79 (m, 1H), 3.44-3.38 (m, 1H), 3.01-2.96 (m, 1H), 2.76 (s, 3H), 2.72-2.69 (m, 1H), 1.97-1.91 (m, 2H), 1.64-1.54 (m, 1H).

### Synthesis of (R)-2-fluoro-N-(8-methylisoquinolin-1-yl)-N-(piperidin-3-yl)-4-((4-(piperidin-4-yl)pyrimidin-2-yl)amino)benzamide (Example 662)

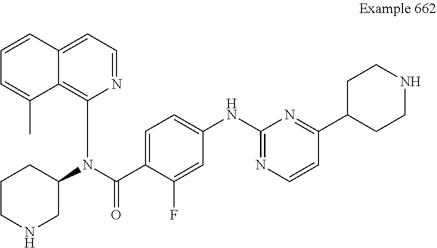 [SMILES: Cc1cccc2ccnc(N(C(=O)c3ccc(Nc4nccc(C5CCNCC5)n4)cc3F)[C@@H]3CCCNC3)c12]

Example 662 was synthesized following the procedure for example 131 by substituting 3-amino-1-methylpyridin-2 (1H)-one with tert-butyl 4-(2-aminopyrimidin-4-yl)piperidine-1-carboxylate to afford the title compound %) as an off-white solid. LC-MS: [M+H⁺]=539.7. ¹H NMR (CD₃OD, 400 MHZ): δ ppm 8.46-8.38 (m, 2H), 7.80-7.75 (m, 1H), 7.71-7.63 (m, 1H), 7.56-7.52 (m, 1H), 7.49-7.47 (m, 1H), 7.36-7.33 (m, 1H), 7.09 (d, J=5.2 Hz, 1H), 6.92-6.90 (m, 1H), 6.87-6.83 (m, 1H), 5.09-5.03 (m, 1H), 3.99-3.97 (m, 1H), 3.85-3.80 (m, 1H), 3.52-3.49 (m, 3H), 3.42-3.38 (m, 1H), 3.19-3.13 (m, 3H), 3.03-2.99 (m, 1H), 2.83 (s, 3H), 2.19-2.15 (m, 3H), 1.98-1.91 (m, 3H), 1.57-1.53 (m, 1H).

### Synthesis of (R)-2-fluoro-N-(8-methylisoquinolin-1-yl)-N-(piperidin-3-yl)-4-((4-(tetrahydro-2H-pyran-4-yl)pyrimidin-2-yl)amino)benzamide (Example 663)

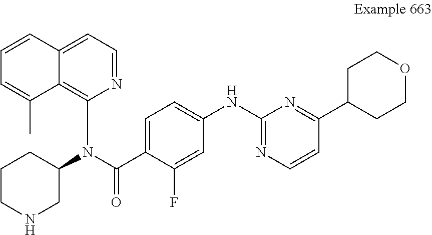 [SMILES: Cc1cccc2ccnc(N(C(=O)c3ccc(Nc4nccc(C5CCOCC5)n4)cc3F)[C@@H]3CCCNC3)c12]

Example 663 was synthesized following the procedure for example 131 by substituting 3-amino-1-methylpyridin-2 (1H)-one with 4-(tetrahydro-2H-pyran-4-yl)pyrimidin-2-amine to afford the title compound as an off-white solid. LC-MS: [M+H⁺]=541.75. ¹H NMR (CD₃OD, 400 MHZ): δ ppm 8.44-8.41 (m, 1H), 8.34 (d, J=5.6 Hz, 1H), 7.76-7.72 (m, 1H), 7.68-7.63 (m, 1H), 7.53-7.45 (m, 2H), 7.33-7.25 (m, 1H), 7.05 (d, J=6.0 Hz, 1H), 6.87 (d, J=8.8 Hz, 1H), 6.82-6.78 (m, 1H), 5.09-5.03 (m, 1H), 4.08-3.96 (m, 3H), 3.85-3.79 (m, 1H), 3.59-3.54 (m, 2H), 3.44-3.38 (m, 1H), 3.03-3.00 (m, 2H), 2.82 (s, 3H), 2.73-2.69 (m, 1H), 1.95-1.92 (m, 2H), 1.89-1.71 (m, 4H), 1.61-1.58 (m, 1H).

### Synthesis of (R)—N-(3-chloro-1-methyl-1H-pyrrolo[2,3-c]pyridin-7-yl)-2-fluoro-4-(5-methyl-1,3,4-thiadiazol-2-yl)-N-(piperidin-3-yl)benzamide (Example 664)

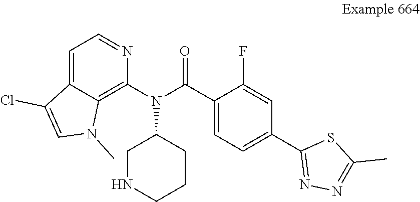 [SMILES: Cc1nnc(-c2ccc(C(=O)N(c3nccc4c(Cl)cn(C)c34)[C@@H]3CCCNC3)c(F)c2)s1]

Example 664 was prepared according to the procedure described for Example 667 by substituting 2-methylthiazolo [4,5-c] pyridine and 2-bromo-5-ethyl-1,3,4-thiadiazole with 3-chloro-1-methyl-1H-pyrrolo[2,3-c]pyridine and 2-bromo-5-methyl-1,3,4-thiadiazole, provided the title compound hydrochloride salt as a white solid. LC-MS: [M+H⁺]=485.1. ¹H NMR (400 MHZ, DMSO-d₆): δ ppm 0.97-1.11 (m, 1H) 1.72-1.89 (m, 3H) 2.73 (s, 4H) 2.83 (s, 1H) 3.16-3.26 (m, 1H) 3.33-3.48 (m, 1H) 3.76 (br d, J=11.13 Hz, 1H) 3.95-4.04 (m, 4H) 4.88-5.00 (m, 1H) 7.20 (t, J=7.57 Hz, 1H) 7.45 (dd, J=8.13, 1.50 Hz, 1H) 7.48 (d, J=5.38 Hz, 1H) 7.59-7.68 (m, 1H) 7.84-7.92 (m, 1H) 7.92-7.92 (m, 1H) 8.14 (d, J=5.38 Hz, 1H) 8.91-9.18 (m, 2H).

### Synthesis of (R)-2-fluoro-N-(8-methylisoquinolin-1-yl)-N-(piperidin-3-yl)-4-((4-(1,2,3,6-tetrahydropyridin-4-yl)pyrimidin-2-yl)amino)benzamide (Example 665)

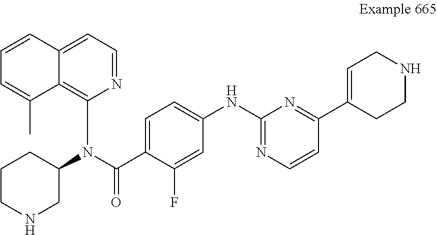 [SMILES: Cc1cccc2ccnc(N(C(=O)c3ccc(Nc4nccc(C5=CCNCC5)n4)cc3F)[C@@H]3CCCNC3)c12]

Example 665 was synthesized following the procedure for example 131 by substituting 3-amino-1-methylpyridin-2(1H)-one tert-butyl 4-(2-aminopyrimidin-4-yl)-3,6-dihydropyridine-1(2H)-carboxylate to afford the title compound as an off-white solid. LC-MS: [M+H⁺]=538.4. ¹H NMR (CD₃OD, 400 MHZ): δ 8.45-8.40 (m, 2H), 7.79-7.74 (m, 1H), 7.70-7.65 (M, 1H), 7.55-7.46 (m, 2H), 7.37-7.29 (m, 2H), 7.06 (s, 1H), 6.89 (d, J=8.4 Hz, 1H), 6.83-6.79 (m, 1H), 5.09-5.03 (m, 1H), 4.00-3.96 (m, 2H), 3.84-3.75 (m, 1H), 3.52-3.47 (m, 3H), 3.41-3.38 (m, 1H), 3.00-2.95 (m, 1H), 2.85-2.69 (m, 6H), 1.95-1.88 (m, 2H), 1.59-1.53 (m, 1H).

### Synthesis of (R)-4-(5-ethyl-1,3,4-thiadiazol-2-yl)-2-fluoro-N-(2-methylthiazolo[4,5-c]pyridin-4-yl)-N-(piperidin-3-yl)benzamide (Example 667)

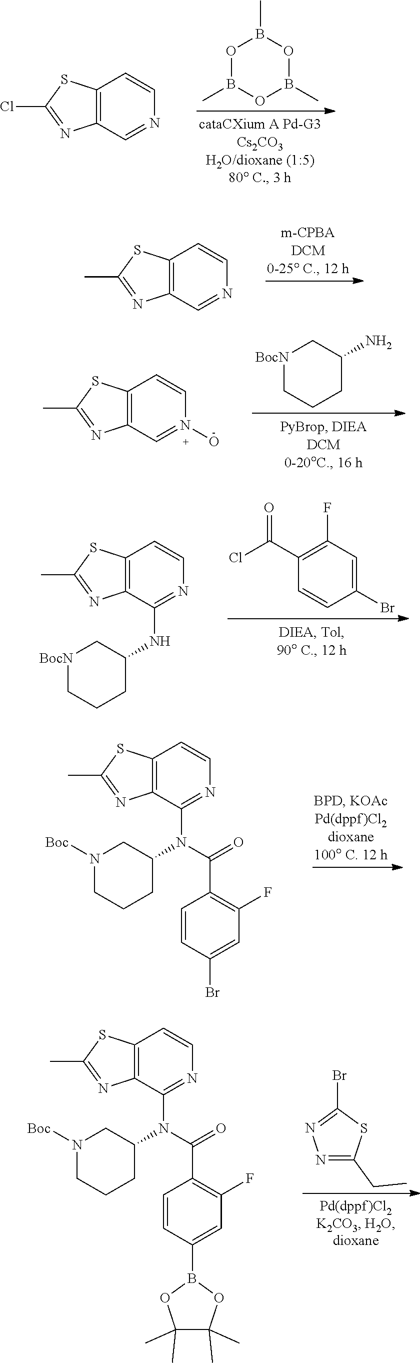

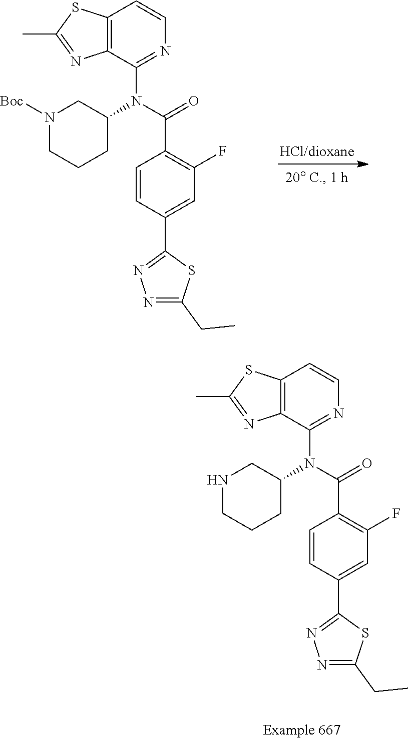

### Step 1:

A mixture of 2-chlorothiazolo[4,5-c]pyridine (0.5 g, 2.93 mmol), Cs₂CO₃ (2.86 g, 8.79 mmol), Pd(PPh₃)₂Cl₂ (205.69 mg, 293.05 μmol) and 2,4,6-trimethyl-1,3,5,2,4,6-trioxatriborinane (735.76 mg) in dioxane (1 mL)/H₂O (5 mL) was degassed with N₂, and then the mixture was stirred at 110° C. for 12 h under N₂ atmosphere. The reaction mixture was filtered and the filtrate was concentrated. The residue was purified by prep-HPLC to give 2-methylthiazolo [4,5-c] pyridine (0.05 g, 11%) as a yellow solid.

### Step 2:

To a solution of 2-methylthiazolo[4,5-c]pyridine (0.05 g, 332.89 μmol) in DCM (1 mL) at 0° C. was added m-CPBA (153.19 mg, 665.78 μmol). The mixture was stirred at 25° C. for 2 h. The reaction mixture was quenched by addition aqueous NaSO₃ solution (3 mL) at 25° C., and then extracted with DCM (60 mL). The combined organic layer was washed with brine, dried over Na₂SO₄, filtered and concentrated to provide the crude 2-methylthiazolo[4,5-c]pyridine 5-oxide was used in the next step without further purification.

### Step 3:

To a solution of crude 2-methylthiazolo[4,5-c]pyridine 5-oxide (0.50 g, 3.01 mmol) in dichloromethane (5 mL) were added tert-butyl (3R)-3-aminopiperidine-1-carboxylate (1.21 g, 6.02 mmol) and DIPEA (1.56 g, 12.03 mmol). The mixture was stirred at 0° C. for 0.5 h and followed by PyBrop (2.10 g, 4.51 mmol). The mixture was stirred at 25° C. for 11.5 h before it was concentrated. The residue was purified by prep-TLC (SiO₂, Petroleum ether/EtOAc=1/1) to give tert-butyl (R)-3-((2-methylthiazolo[4,5-c]pyridin-4-yl)amino)piperidine-1-carboxylate (0.37 g, 35%) as a white solid.

### Steps 4:

To a solution of 4-bromo-2-fluoro-benzoyl chloride (408.86 mg, 1.72 mmol) in toluene (3 mL) was added DIPEA (333.80 mg, 2.58 mmol) and tert-butyl (R)-3-((2-methylthiazolo[4,5-c]pyridin-4-yl)amino)piperidine-1-carboxylate (300 mg, 860.93 μmol). The mixture was stirred at 90° C. for 12 h. The reaction mixture was concentrated to provide crude tert-butyl (R)-3-(4-bromo-2-fluoro-N-(2-methylthiazolo[4,5-c]pyridin-4-yl)benzamido)piperidine-1-carboxylate (290 mg, 61%) as a white solid.

### Steps 5-7:

Example 667 was prepared according to the procedure described for Example 699 by substituting tert-butyl (3R)-3-[(2-fluoro-4-iodo-benzoyl)-(3-methylthieno[3,2-c]pyridin-4-yl)amino]piperidine-1-carboxylate and 2-bromo-5-methyl-1,3,4-oxadiazole with tert-butyl (R)-3-(4-bromo-2-fluoro-N-(2-methylthiazolo[4,5-c]pyridin-4-yl)benzamido)piperidine-1-carboxylate and 2-bromo-5-ethyl-1,3,4-thiadiazole, provided the title compound hydrochloride salt as a white solid. LC-MS: [M+H⁺]=483.1. ¹H NMR (400 MHZ, CD₃OD): δ ppm 8.35 (d, J=5.4 Hz, 1H), 7.91 (d, J=5.5 Hz, 1H), 7.50 (br d, J=10.3 Hz, 1H), 7.43 (br d, J=8.4 Hz, 1H), 7.30 (br t, J=7.3 Hz, 1H), 5.07 (br s, 1H), 3.83 (br d, J=12.1 Hz, 1H), 3.59 (br t, J=11.4 Hz, 1H), 3.36 (br s, 1H), 3.14 (q, J=7.5 Hz, 2H), 2.94-2.79 (m, 4H), 2.18-2.07 (m, 1H), 2.00-1.79 (m, 2H), 1.77-1.59 (m, 1H), 1.40 (t, J=7.6 Hz, 3H).

### Synthesis of N-(3-chlorothieno[3,2-c]pyridin-4-yl)-5-(5-ethyl-1,3,4-thiadiazol-2-yl)-N-[(3R)-3-piperidyl]pyridine-2-carboxamide (Example 668)

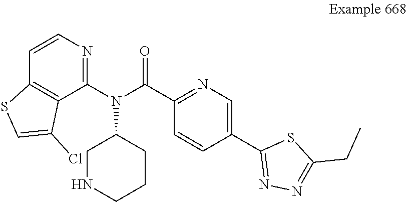 [SMILES: CCc1nnc(-c2ccc(C(=O)N(c3nccc4scc(Cl)c34)[C@@H]3CCCNC3)nc2)s1]

Example 668 was prepared according to the procedure described for Example 127 by substituting tert-butyl (R)-3-((8-methylisoquinolin-1-yl)amino)piperidine-1-carbo-xylate, 2-fluoro-4-iodobenzoyl chloride and 2-bromo-5-methyl-1,3,4-thiadiazole with tert-butyl (3R)-3-[(3-chlorothieno[3, 2-c] pyridin-4-yl)amino]piperidine-1-carboxylate, 5-bromo-pyridine-2-carbonyl chloride and 2-bromo-5-ethyl-1,3,4-thiadiazole, provided the title compound hydrochloride salt as an off-white solid. LC-MS: [M+H⁺]=485.2. ¹H NMR (400 MHZ, CD₃OD): δ ppm 8.53-8.46 (m, 1H), 8.42 (d, J=1.5 Hz, 1H), 8.35-8.24 (m, 1H), 8.16-8.08 (m, 1H), 8.06-7.97 (m, 1H), 7.80-7.69 (m, 1H), 5.15 (tt, J=3.8, 11.8 Hz, 1H), 3.92 (br dd, J=3.7, 12.1 Hz, 1H), 3.72-3.60 (m, 1H), 3.36 (br d, J=13.4 Hz, 1H), 3.16 (q, J=7.5 Hz, 2H), 2.91 (dt, J=3.6, 12.5 Hz, 1H), 2.17-1.81 (m, 3H), 1.41 (t, J=7.5 Hz, 3H), 1.36-1.19 (m, 1H).

### Synthesis of 5-(5-ethyl-1,3,4-thiadiazol-2-yl)-N-(3-methylthieno[2,3-c]pyridin-7-yl)-N-[(3R)-3-piperidyl]pyridine-2-carboxamide (Example 669)

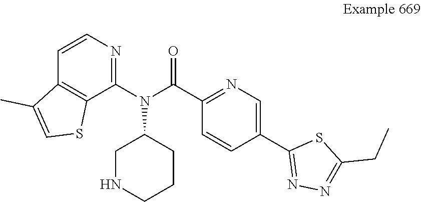 [SMILES: CCc1nnc(-c2ccc(C(=O)N(c3nccc4c(C)csc34)[C@@H]3CCCNC3)nc2)s1]

Example 669 was prepared according to the procedure described for Example 668 by substituting tert-butyl (R)-3-((3-chlorothieno[3,2-c]pyridin-4-yl)amino)piperidine-1-carboxylate with tert-butyl (R)-3-((3-methylthieno[2,3-c]pyridin-7-yl)amino)piperidine-1-carboxylate to afford the title compound as a dark brown solid. LC-MS: [M+H⁺]=465.2. ¹H NMR (400 MHz, DMSO-d₆): δ=9.36-8.90 (m, 2H), 8.73-8.21 (m, 3H), 8.00-7.88 (m, 1H), 7.87-7.70 (m, 2H), 5.18-4.97 (m, 1H), 3.74-3.35 (m, 2H), 3.31-3.14 (m, 3H), 2.88-2.66 (m, 1H), 2.49-2.36 (m, 3H), 1.84 (br d, J=14.8 Hz, 3H), 1.45-1.25 (m, 4H).

### Synthesis of (R)-5-(5-ethyl-1,3,4-thiadiazol-2-yl)-N-(3-methylthieno[3,2-c]pyridin-4-yl)-N-(piperidin-3-yl)picolinamide (Example 670)

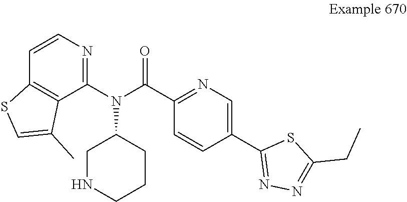 [SMILES: CCc1nnc(-c2ccc(C(=O)N(c3nccc4scc(C)c34)[C@@H]3CCCNC3)nc2)s1]

Example 670 was prepared according to the procedure described for Example 668 by substituting tert-butyl (R)-3-((3-chlorothieno[3,2-c]pyridin-4-yl)amino)piperidine-1-carboxylate with tert-butyl (R)-3-((3-methylthieno[2,3-c]pyridin-4-yl)amino)piperidine-1-carboxylate to afford the title compound as an off-white solid. LC-MS: [M+H⁺]=465.2. ¹H NMR (400 MHZ, DMSO-d₆): δ=9.14-8.89 (m, 2H), 8.54-8.41 (m, 1H), 8.32 (d, J=5.5 Hz, 1H), 8.27-8.17 (m, 1H), 8.06-7.94 (m, 1H), 7.73-7.62 (m, 1H), 7.57-7.48 (m, 1H), 5.00-4.83 (m, 1H), 3.78-3.73 (m, 1H), 3.53-3.39 (m, 1H), 3.22 (br d, J=7.4 Hz, 1H), 3.14-3.08 (m, 2H), 2.84-2.65 (m, 2H), 2.57 (br d, J=11.3 Hz, 2H), 1.83-1.65 (m, 3H), 1.30 (t, J=7.5 Hz, 3H), 1.10-0.98 (m, 1H).

### Synthesis of (R)—N-(6,8-dimethylisoquinolin-1-yl)-5-(5-ethyl-1,3,4-thiadiazol-2-yl)-N-(piperidin-3-yl)picolinamide (Example 671)

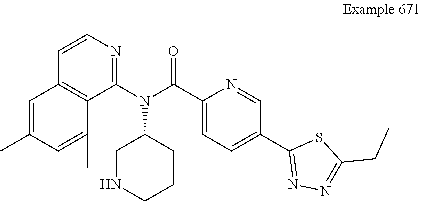 [SMILES: CCc1nnc(-c2ccc(C(=O)N(c3nccc4cc(C)cc(C)c34)[C@@H]3CCCNC3)nc2)s1]

Example 671 was prepared according to the procedure described for Example 701 by substituting 2-bromo-5-methyl-1,3,4-thiadiazole with 2-bromo-5-ethyl-1,3,4-thiadiazole, provided the title compound hydrochloride salt as an off-white solid. LC-MS: [M+H⁺]=473.3. ¹H NMR (400 MHZ, CD₃OD): δ ppm 8.47-8.41 (m, 2H), 8.35 (d, J=1.5 Hz, 1H), 8.33-8.25 (m, 2H), 7.92 (s, 1H), 7.67-7.60 (m, 1H), 5.08-4.97 (m, 1H), 4.07 (br d, J=11.8 Hz, 1H), 3.47 (br t, J=11.6 Hz, 1H), 3.36 (br s, 1H), 3.16 (q, J=7.5 Hz, 2H), 2.96-2.87 (m, 1H), 2.83-2.74 (m, 1H), 2.70 (s, 3H), 2.61-2.56 (m, 3H), 1.94-1.83 (m, 2H), 1.70 (br d, J=11.9 Hz, 1H), 1.40 (t, J=7.6 Hz, 3H), 1.11-0.95 (m, 1H).

### Synthesis of (R)-5-(5-ethyl-1,3,4-thiadiazol-2-yl)-N-(6-fluoro-8-methylisoquinolin-1-yl)-N-(piperidin-3-yl)picolinamide (Example 672)

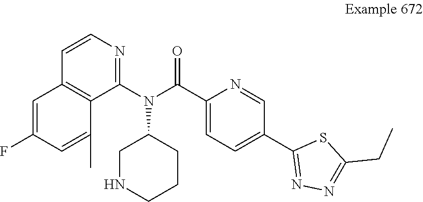 [SMILES: CCc1nnc(-c2ccc(C(=O)N(c3nccc4cc(F)cc(C)c34)[C@@H]3CCCNC3)nc2)s1]

Example 672 was prepared according to the procedure described for Example 701 by substituting tert-butyl (3R)-3-[(6,8-dimethyl-1-isoquinolyl) amino] piperidine-1-carboxylate and 2-bromo-5-methyl-1,3,4-thiadiazole with tert-butyl (3R)-3-[(6-fluoro-8-methyl-1-isoquinolyl)amino]piperidine-1-carboxylate and 2-bromo-5-ethyl-1,3,4-thiadiazole, provided the title compound hydrochloride salt as an off-white solid. LC-MS: [M+H⁺]=477.2. ¹H NMR (400 MHZ, DMSO-d₆): δ ppm 8.81 (s, 2H), 8.41-8.19 (m, 3H), 7.92-7.77 (m, 2H), 7.65 (dd, J=2.7, 8.9 Hz, 1H), 7.53-7.38 (m, 1H), 4.90-4.71 (m, 1H), 3.44-3.31 (m, 1H), 3.26-3.18 (m, 1H), 3.11 (q, J=7.5 Hz, 2H), 2.96-2.87 (m, 1H), 2.83-2.70 (m, 4H), 1.83-1.63 (m, 3H), 1.30 (t, J=7.5 Hz, 3H), 1.07-0.91 (m, 1H).

### Synthesis of 5-(5-ethyl-1,3,4-thiadiazol-2-yl)-N-(8-methyl-1-isoquinolyl)-N-[(3R)-3-piperidyl]pyridine-2-carboxamide (Example 673)

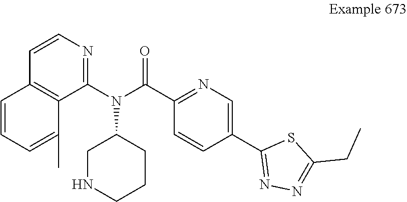 [SMILES: CCc1nnc(-c2ccc(C(=O)N(c3nccc4cccc(C)c34)[C@@H]3CCCNC3)nc2)s1]

Example 673 was prepared according to the procedure described for Example 668 by substituting tert-butyl (R)-3-((3-chlorothieno[3,2-c]pyridin-4-yl)amino)piperidine-1-carboxylate with tert-butyl (R)-3-((8-methylisoquinolin-1-yl)amino)piperidine-1-carboxylate to afford the title compound as a yellow solid. LC-MS: [M+H⁺]=459.3. ¹H NMR (400 MHZ, CD₃OD): δ=8.48-8.29 (m, 3H), 8.27-8.05 (m, 2H), 8.05-7.91 (m, 1H), 7.90-7.71 (m, 1H), 7.71-7.56 (m, 1H), 5.10-4.96 (m, 1H), 4.04 (br d, J=9.8 Hz, 1H), 3.59-3.44 (m, 1H), 3.35 (br s, 1H), 3.15 (q, J=7.5 Hz, 2H), 3.00-2.85 (m, 2H), 2.79 (br s, 2H), 1.88 (br d, J=3.4 Hz, 2H), 1.79 (br d, J=12.8 Hz, 1H), 1.40 (t, J=7.6 Hz, 3H), 1.09 (br d, J=5.6 Hz, 1H).

### Synthesis of (R)—N-(3-ethynylthieno[3,2-c]pyridin-4-yl)-2-fluoro-4-(1-methyl-1H-1,2,3-triazol-4-yl)-N-(piperidin-3-yl)benzamide (Example 674)

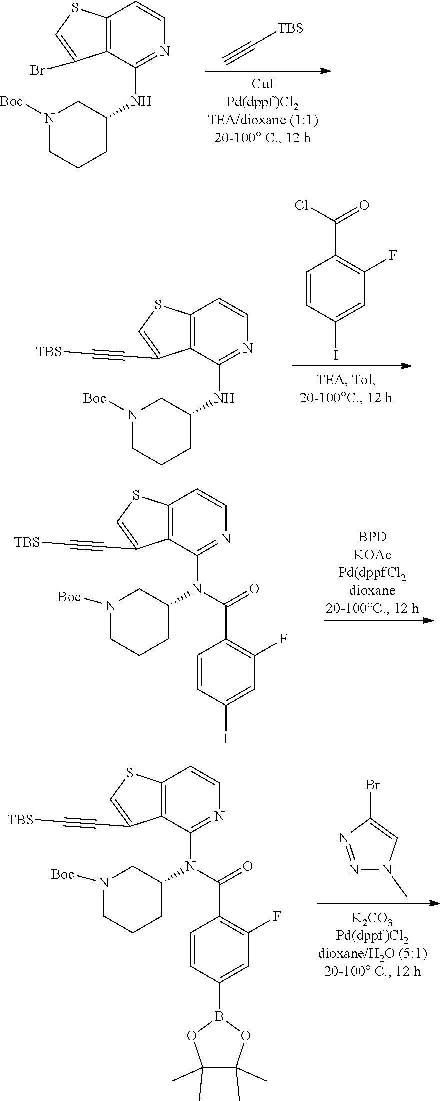

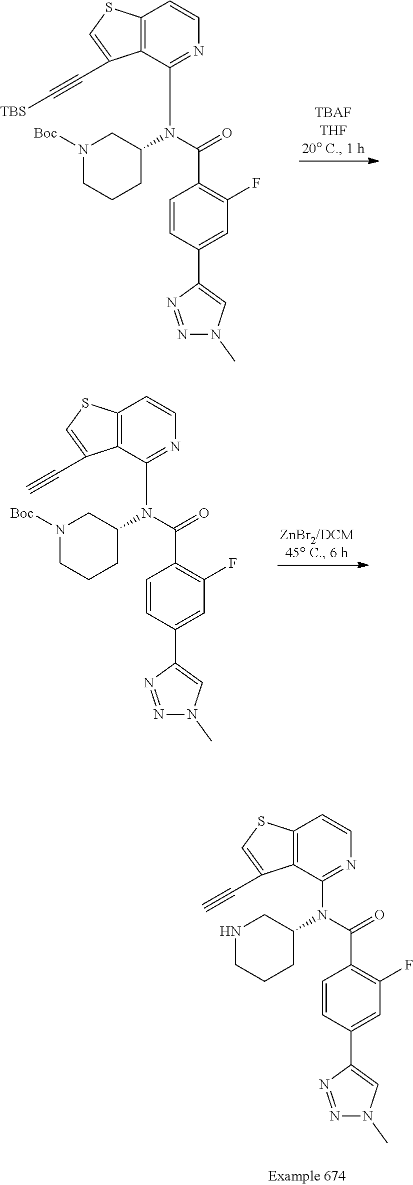

### Step 1:

To a solution of tert-butyl (R)-3-((3-bromothieno[3,2-c]pyridin-4-yl)amino)piperidine-1-carboxylate (500 mg, 1.09 mmol) in dioxane (2.5 mL) was added tert-butyl-ethynyl-dimethyl-silane (1.07 g, 7.64 mmol), Pd(dppf)Cl₂ (159.70 mg, 218.26 μmol), CuI (31.18 mg, 163.70 μmol) and TEA (2.5 mL) at 20° C. under N₂ atmosphere, then the mixture was stirred at 100° C. for 2 h. The reaction mixture was diluted with (3 mL), then extracted with EtOAc (3×5 mL). The combined organic layer was washed with brine (3×5 mL), dried over Na₂SO₄, filtered and concentrated. The residue was purified by column chromatography (SiO₂, Petroleum ether/EtOAc=20/1 to 5/1) to give tert-butyl (R)-3-((3-((tert-butyldimethylsilyl) ethynyl)thieno[3,2-c]pyridin-4-yl)amino)piperidine-1-carboxylate (420 mg, 77%) as an off-white solid.

### Step 2:

To a solution of 2-fluoro-4-iodo-benzoyl chloride (381.90 mg, 1.21 mmol) in toluene (5 mL) were added TEA (391.26 mg, 3.87 mmol, 538.18 μL) and tert-butyl (R)-3-((3-((tert-butyldimethylsilyl)ethynyl)thieno [3,2-c]pyridin-4-yl)amino)piperidine-1-carboxylate (120 mg, 241.66 μmol) at 20° C. under N₂ atmosphere, then the mixture was stirred at 100° C. for 12 h. The reaction mixture was diluted with water (10 mL), then extracted with ethyl acetate (3×20 mL). The combined organic layer was washed with brine (3×20 mL), dried over Na₂SO₄, filtered and concentrated. The residue was purified by column chromatography (SiO₂, Petroleum ether/EtOAc=30/1 to 15/1) to give tert-butyl (R)-3-(N-(3-((tert-butyldimethylsilyl)ethynyl)thieno[3,2-c]pyridin-4-yl)-2-fluoro-4-iodobenzamido)piperidine-1-carboxylate (620 mg, 71%) as brown solid. LC-MS: [M+H⁺]=720.2 Step 3:

To a solution of tert-butyl (R)-3-(N-(3-((tert-butyldimethylsilyl)ethynyl)thieno[3,2-c]pyridin-4-yl)-2-fluoro-4-iodobenzamido)piperidine-1-carboxylate (390 mg, 379.31 μmol) in dioxane (10 mL) was added BPD (482 mg, 1.90 mmol), KOAc (186 mg, 1.90 mmol) and Pd(dppf)Cl₂ (55.51 mg, 75.86 μmol) at 20° C. under N₂ atmosphere, then the mixture was stirred at 100° C. for 12 h. The reaction was diluted with H₂O (10 mL), then extracted with EtOAc (3×15 mL). The combined organic layer was washed with brine (3×15 mL), dried over Na₂SO₄, filtered and concentrated. The residue was purified by prep-TLC (SiO₂, petroleum ether:EtOAc=1:1) to give tert-butyl (R)-3-(N-(3-((tert-butyldimethylsilyl)-ethynyl)thieno [3,2-c]pyridin-4-yl)-2-fluoro-4-(4,4,5,5-tetramethyl-1,3,2-dioxaborolan-2-yl)benzamido)piperidine-1-carboxylate (0.29 g, 88%) as a brown solid. LC-MS: [M+H⁺]=720.5

### Step 4:

To a solution of tert-butyl (R)-3-(N-(3-((tert-butyldimethylsilyl)ethynyl)thieno[3,2-c]pyridin-4-yl)-2-fluoro-4-(4,4,5,5-tetramethyl-1,3,2-dioxaborolan-2-yl)benzamido)-piperidine-1-carboxylate (170 mg, 196.03 μmol) in dioxane (10 mL) were added 4-bromo-1-methyl-triazole (95.26 mg, 588.09 μmol), K₂CO₃ (81.28 mg, 588.09 μmol), Pd(dppf)Cl₂ (28.69 mg, 39.21 μmol) and H₂O (0.3 mL) at 20° C. under N₂ atmosphere, then the mixture was stirred at 100° C. for 1 h. The reaction mixture was diluted with water (15 mL), then extracted with EtOAc (3×20 mL). The combined organic layer was washed with brine (3×20 mL), dried over Na₂SO₄, filtered and concentrated. The residue was purified by column chromatography (SiO₂, Petroleum ether/tetrahydrofuran=20/1 to 5/1) to give tert-butyl (R)-3-(N-(3-((tert-butyldimethylsilyl)ethynyl)thieno[3,2-c]pyridin-4-yl)-2-fluoro-4-(1-methyl-1H-1,2,3-triazol-4-yl)benzamido)piperidine-1-carboxylate (300 mg, 70%) as a brown solid. LC-MS: [M+H⁺]=675.4

### Step 5:

To a solution of tert-butyl (R)-3-(N-(3-((tert-butyldimethylsilyl)ethynyl)thieno[3,2-c]pyridin-4-yl)-2-fluoro-4-(1-methyl-1H-1,2,3-triazol-4-yl)benzamido)piperidine-1-carboxylate (80 mg, 110.24 μmol) in THF (2 mL) was added TBAF (86.47 mg, 330.71 μmol) at 20° C. under N₂ atmosphere, then the mixture was stirred at 20° C. for 1 h. The reaction mixture was diluted with water (10 mL) and then extracted with EtOAc (3×20 mL). The combined organic layer was washed with brine (3×20 mL), dried over Na₂SO₄, filtered and concentrated. The residue was purified by prep-TLC (SiO₂, petroleum ether:EtOAc=1:6) to give tert-butyl (R)-3-(N-(3-ethynylthieno[3,2-c]pyridin-4-yl)-2-fluoro-4-(1-methyl-1H-1,2,3-triazol-4-yl)benzamido)piperi-dine-1-carboxylate (90 mg, 47%) as an off-white solid. LC-MS: [M+H⁺]=561.3

### Step 6:

A solution of tert-butyl (R)-3-(N-(3-ethynylthieno[3,2-c]pyridin-4-yl)-2-fluoro-4-(1-methyl-1H-1,2,3-triazol-4-yl)benzamido)piperidine-1-carboxylate (60 mg, 99.53 μmol) in DCM (4 mL) was added ZnBr₂ (112.07 mg, 497.64 μmol, 24.90 μL) at 20° C. under N₂ atmosphere, then the mixture was stirred at 45° C. for 6 h. The reaction mixture was filtered and concentrated under reduced pressure to give a residue. The residue was purified by prep-HPLC to give the title compound (25 mg, 36%) as a white solid. LC-MS: [M+H⁺]=461.3. ¹H NMR (400 MHZ, DMSO-d₆): δ ppm 8.42 (s, 1H), 8.40-8.34 (m, 1H), 8.27-8.22 (m, 1H), 8.03-7.99 (m, 1H), 7.33-7.27 (m, 2H), 7.25-7.17 (m, 1H), 4.81-4.66 (m, 1H), 4.56-4.44 (m, 1H), 4.01 (s, 3H), 3.42 (br d, J=11.4 Hz, 1H), 3.02-2.87 (m, 1H), 2.78 (br d, J=9.4 Hz, 1H), 2.42-2.27 (m, 1H), 2.23-2.13 (m, 1H), 1.86-1.73 (m, 1H), 1.57-1.34 (m, 2H), 0.72 (dq, J=4.3, 12.1 Hz, 1H).

### Synthesis of 2-fluoro-N-(3-isopropenylthieno[2,3-c]703yridine-7-yl)-4-(1-methyltriazol-4-yl)-N-[(3R)-3-piperidyl]benzamide (Example 675)

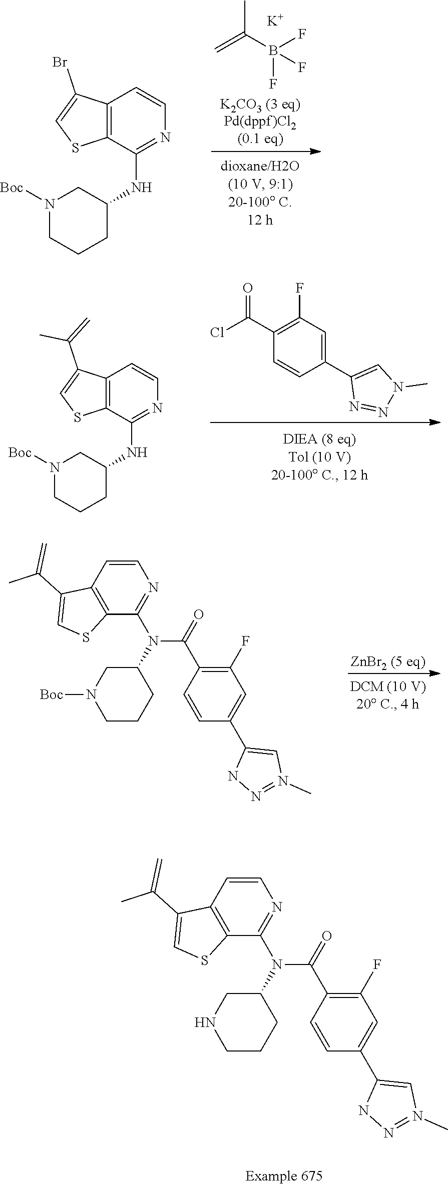

To a solution of tert-butyl (3R)-3-[(3-bromothieno[2,3-c]704yridine-7-yl)amino]piperidine-1-carboxylate (80 mg, 184.31 μmol, 1 eq) in dioxane (0.9 mL) and H₂O (0.1 mL) was added K₂CO₃ (76.42 mg, 552.94 μmol, 3 eq), potassium trifluoro(isopropenyl)borate (54.55 mg, 368.62 μmol, 2 eq) and Pd(dppf)Cl₂ (13.49 mg, 18.43 μmol, 0.1 eq) at 20° C. under N₂. The mixture was stirred at 100° C. for 12 hours. The reaction mixture was quenched with water (20 mL) and extracted with EtOAc (3×20 mL). The combined organic layers were washed with brine (3×60 mL), dried over Na₂SO₄, filtered and concentrated under reduced pressure to give the crude product. The crude product was purified by prep-TLC using 50% EtOAc in petroleum ether to give tert-butyl (3R)-3-[(3-isopropenylthieno[2,3-c]704yridine-7-yl)amino]piperidine-1-carboxylate (40 mg, 52%) as yellow oil. LC-MS: [M+H]=374.2.

### Step 2:

To a solution of tert-butyl (3R)-3-[(3-isopropenylthieno[2,3-c]704yridine-7-yl)amino]piperidine-1-carboxylate (40 mg, 96.38 μmol, 1 eq) in Tol. (1 mL) was added TEA (78.02 mg, 771.06 μmol, 107.32 μL, 8 eq) and 2-fluoro-4-(1-methyltriazol-4-yl)benzoyl chloride (57.74 mg, 192.77 μmol, 2 eq) at 20° C. under N₂. The mixture was purged with N₂ for three times. The mixture was stirred at 100° C. for 12 hours. The reaction mixture was quenched with water (20 mL) at 0° C., then extracted with EtOAc (3×20 mL). The combined organic layers were washed with brine (3×30 mL), dried over Na₂SO₄, filtered and concentrated under reduced pressure to give the crude product. The crude product was purified by prep-TLC using EtOAc to give the tert-butyl (3R)-3-[[2-fluoro-4-(1-methyltriazol-4-yl)benzoyl]-(3-isopropenylthieno[2,3-c]704yridine-7-yl)amino]piperidine-1-carboxylate (40 mg, 43%) as off white solid. LC-MS: [M+H]=577.3.

### Step 3:

Removal of the Boc protecting was followed as described for Example 674 to provide the title compound as an off-white solid. LC-MS: [M+H⁺]=477.3. ¹H NMR (400 MHZ, DMSO-d₆): δ=8.37-8.57 (m, 2H), 8.07 (s, 1H), 7.83-7.96 (m, 1H), 6.95 (br d, J=15.13 Hz, 3H), 5.28-5.43 (m, 2H), 4.00-4.09 (m, 3H), 3.13-3.20 (m, 1H), 2.64-2.79 (m, 2H), 2.33 (br s, 1H), 2.07-2.22 (m, 5H), 1.80-2.01 (m, 1H), 1.40-1.61 (m, 2H).

### Synthesis of N-(3-ethynylthieno[2,3-c]704yridine-7-yl)-2-fluoro-4-(1-methyltriazol-4-yl)-N-[(3R)-3-piperidyl]benzamide (Example 676)

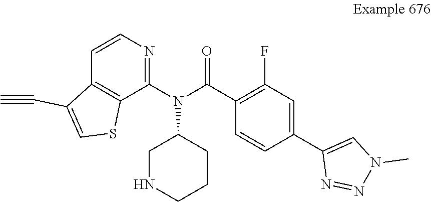 [SMILES: C#Cc1csc2c(N(C(=O)c3ccc(-c4cn(C)nn4)cc3F)[C@@H]3CCCNC3)nccc12]

Example 676 was prepared according to the procedure described for Example 674 by substituting tert-butyl (R)-3-((3-bromothieno[3,2-c]pyridin-4-yl)amino)piperidine-1-carboxylate with tert-butyl (3R)-3-[(3-bromothieno[2,3-c]705yridine-7-yl)amino]piperidine-1-carboxylate, provided the title compound as an off-white solid. LC-MS: [M+H]=462.2. ¹H NMR (400 MHZ, DMSO-d₆): δ=8.35-8.63 (m, 3H), 7.72 (br s, 1H), 6.97-7.60 (m, 3H), 4.56 (s, 1H), 4.04 (s, 3H), 3.17 (br d, J=5.25 Hz, 1H), 2.66-2.80 (m, 1H), 2.07-2.37 (m, 2H), 1.83-2.03 (m, 1H), 1.21-1.67 (m, 3H).

### Synthesis of (R)-2-fluoro-4-(1-methyl-1H-1,2,3-triazol-4-yl)-N-(3-methylthieno[2,3-c]pyridin-7-yl)-N-(piperidin-3-yl)benzamide (Example 677)

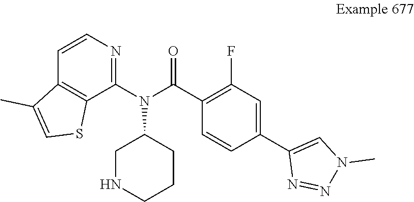

Example 677 was prepared according to the procedure described for Example 127 by substituting tert-butyl (R)-3-((8-methylisoquinolin-1-yl)amino)piperidine-1-carboxylate with tert-butyl (R)-3-((3-methylthieno[2,3-c]pyridin-7-yl)amino)piperidine-1-carboxylate and 2-bromo-5-methyl-1,3,4-thiadiazole with 4-bromo-1-methyl-1H-1,2,3-triazole, provided the title compound as a white solid. LC-MS: [M+H]=451.2. ¹H NMR (400 MHZ, DMSO-d6): δ=9.90-10.28 (m, 1H), 9.00-9.23 (m, 1H), 8.74-8.97 (m, 1H), 8.35-8.58 (m, 2H), 7.91 (br dd, J=6.00, 5.00 Hz, 1H), 7.65-7.82 (m, 2H), 7.39-7.52 (m, 1H), 7.23-7.37 (m, 1H), 7.07-7.22 (m, 1H), 4.61-5.21 (m, 1H), 3.96-4.10 (m, 3H), 3.12-3.22 (m, 1H), 2.63-2.71 (m, 1H), 1.85-1.97 (m, 1H), 1.75 (br s, 2H), 0.99-1.32 (m, 2H).

### Synthesis of N-(3-chlorothieno[2,3-c]705yridine-7-yl)-2-fluoro-4-(1-methyltriazol-4-yl)-N-[(3R)-3-piperidyl]benzamide (Example 678)

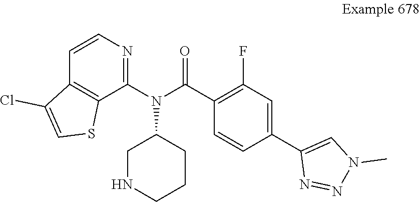 [SMILES: Cn1cc(-c2ccc(C(=O)N(c3nccc4c(Cl)csc34)[C@@H]3CCCNC3)c(F)c2)nn1]

Example 678 was prepared according to the procedure described for Example 127 by substituting tert-butyl (R)-3-((8-methylisoquinolin-1-yl)amino)piperidine-1-carboxylate with tert-butyl (R)-3-((3-chlorothieno[2,3-c]pyridin-7-yl)amino)piperidine-1-carboxylate and 2-bromo-5-methyl-1,3,4-thiadiazole with 4-bromo-1-methyl-1H-1,2,3-triazole, provided the title compound hydrochloride salt as a white solid. LC-MS: [M+H]=471.1. ¹H NMR (400 MHz, DMSO-d6): δ=8.96-9.06 (m, 1H), 8.71-8.85 (m, 1H), 8.60 (br d, J=5.13 Hz, 1H), 8.51 (br s, 1H), 8.30 (s, 1H), 7.79 (br d, J=4.75 Hz, 1H), 7.45-7.56 (m, 1H), 7.32-7.44 (m, 1H), 7.12-7.27 (m, 1H), 4.72-5.14 (m, 1H), 4.04 (s, 3H), 3.56-3.67 (m, 1H), 3.11-3.22 (m, 2H), 2.61-2.74 (m, 1H), 1.87-2.03 (m, 1H), 1.65-1.85 (m, 3H), 1.13-1.26 (m, 1H).

### Synthesis of (R)-2-fluoro-4-((4-(4-hydroxycyclohexyl)pyrimidin-2-yl)amino)-N-(8-methylisoquinolin-1-yl)-N-(piperidin-3-yl)benzamide (Example 679)

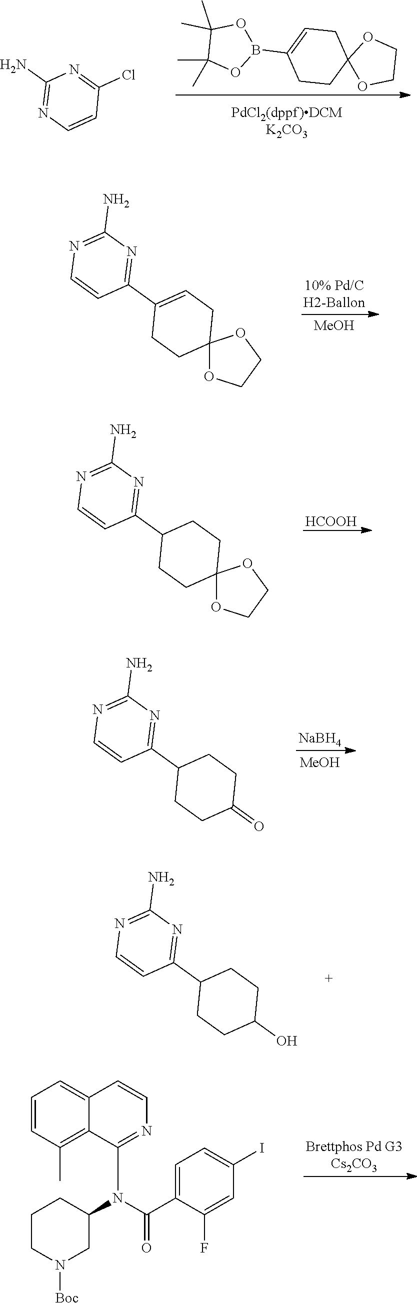

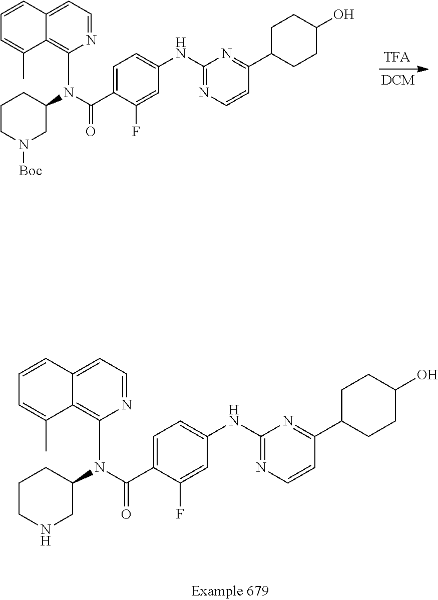 [SMILES: Cc1cccc2ccnc(N(C(=O)c3ccc(Nc4nccc(C5CCC(O)CC5)n4)cc3F)[C@@H]3CCCN(C(=O)OC(C)(C)C)C3)c12 ; Cc1cccc2ccnc(N(C(=O)c3ccc(Nc4nccc(C5CCC(O)CC5)n4)cc3F)[C@@H]3CCCNC3)c12]

### Step 1:

To a stirred solution of 4-chloropyrimidin-2-amine (1 g, 7.72 mmol) and 4,4,5,5-tetramethyl-2-(1,4-dioxaspiro[4.5]dec-7-en-8-yl)-1,3,2-dioxaborolane (3.08 g, 11.58 mmol) in 1,4-dioxane/water (3:1) (24 mL) was added K₂CO₃ (3.20 g, 23.16 mmol) and the reaction mixture was degassed with nitrogen for 10 mins. Pd(dppf)Cl₂.DCM (630 mg, 0.772 mmol) was added to reaction mixture and stirred at 100° C. for 16 h. The reaction was diluted with H₂O (50 mL) and extracted with EtOAc (2×150 mL). The combined organic layer was washed with brine, dried over Na₂SO₄, and concentrated. The obtained compound was purified by Combi-flash using 60-70% EtOAc in hexane to afford 4-(1,4-dioxaspiro[4.5]-dec-7-en-8-yl)pyrimidin-2-amine (900 mg, 47%) as an off-white solid. LC-MS: [M+H⁺]=234.1.

### Step 2:

To a stirred solution of 4-(1,4-dioxaspiro[4.5]dec-7-en-8-yl)pyrimidin-2-amine (250 mg, 1.072 mmol) in MeOH (10 mL) was added 10% Pd/C (50 mg, 0.047 mmol) and the reaction mixture was stirred under a hydrogen balloon 40 psi for 16 h at room temperature. The reaction mixture was filtered through celite bed, washed with MeOH (30 mL) and the filtrate was concentrated under reduced pressure to afford 4-(1,4-dioxaspiro[4.5]decan-8-yl)pyrimidin-2-amine (230 mg, 86%) as an off-white solid. LC-MS: [M+H⁺]=236.1.

### Step 3:

A solution of 4-(1,4-dioxaspiro[4.5]decan-8-yl)pyrimidin-2-amine (230 mg, 0.978 mmol) in Formic acid (2.30 mL, 61.0 mmol) was stirred at room temperature for 5 h. The reaction mixture was concentrated. The obtained compound was dissolved in 10% MeOH in DCM (30 mL), basified with saturated NaHCO₃ solution, and extracted with 10% MeOH in DCM (3×50 mL). The combined organic layer was washed with brine (20 mL), dried over sodium sulphate, and concentrated under reduced pressure to afford 4-(2-aminopyrimidin-4-yl)cyclohexan-1-one (110 mg, 56%) as an off-white solid. LC-MS: [M+H⁺]=192.1.

### Step 4:

To a stirred solution of 4-(2-aminopyrimidin-4-yl)cyclohexan-1-one (110 mg, 0.575 mmol) in MeOH (5 mL) was added sodium borohydride (32.6 mg, 0.863 mmol) at 0° C. and the mixture was stirred at 25° C. for 1 h. The reaction was quenched with ice cold water (10 mL) and extracted with 10% MeOH in DCM (3×30 mL). The combined organic layer was dried over Na₂SO₄ and concentrated under reduced pressure to afford 4-(2-aminopyrimidin-4-yl)cyclohexan-1-ol (100 mg, 82%) as an off-white solid. LC-MS: [M+H⁺]=194.1.

### Steps 5-6:

Example 679 was prepared following the procedure described for example 131 by substituting 3-amino-1-methylpyridin-2 (1H)-one with 4-(2-aminopyrimidin-4-yl)cyclohexan-1-one to afford the title compound as an off-white solid. LC-MS: [M+H⁺]=555.7. ¹H NMR (CD₃OD, 400 MHZ): δ 8.43-8.39 (m, 1H), 8.26 (d, J=5.2 Hz, 1H), 7.73 (d, J=5.2 Hz, 1H), 7.66-7.62 (m, 1H), 7.58-7.42 (m, 3H), 6.82-6.79 (m, 1H), 6.72 (d, J=5.2 Hz, 1H), 6.63-6.58 (m, 1H), 5.05-5.02 (m, 1H), 3.92-3.89 (m, 1H), 3.78-3.75 (m, 1H), 3.58-3.54 (m, 1H), 3.35-3.31 (m, 1H), 2.92-2.91 (m, 1H), 2.80 (s, 3H), 2.51-2.47 (m, 1H), 2.07-2.04 (m, 2H), 1.93-1.85 (m, 5H), 1.62-1.53 (m, 3H), 1.42-1.33 (m, 2H).

### Synthesis of (R)-2-fluoro-4-((4-(3-hydroxyazetidin-1-yl)pyrimidin-2-yl)amino)-N-(8-methylisoquinolin-1-yl)-N-(piperidin-3-yl)benzamide (Example 680)

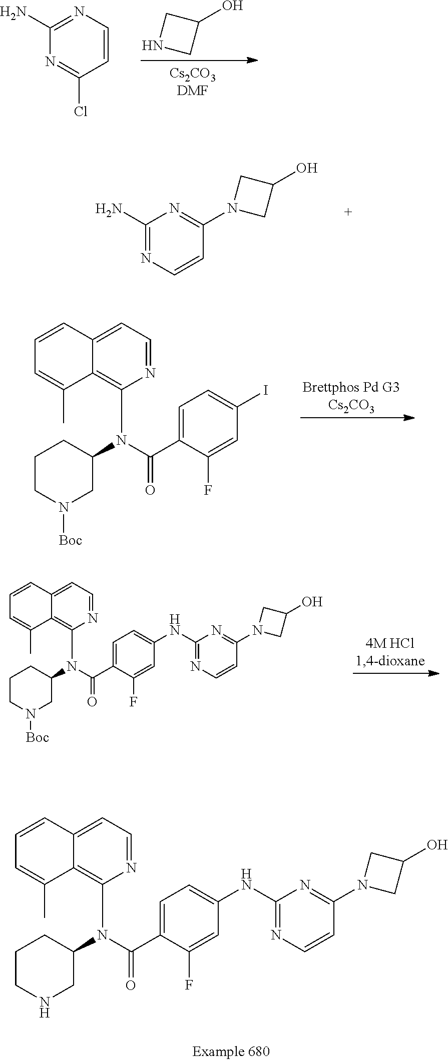

### Step 1:

To a solution of 4-chloropyrimidin-2-amine (350 mg, 2.70 mmol) and azetidin-3-ol hydrochloride (444 mg, 4.05 mmol) in DMF (5 mL) was added Cs₂CO₃ (1320 mg, 4.05 mmol) at room temperature. The reaction mixture was irradiated with microwave at 120° C. for 2 h. The reaction mixture was concentrated and obtained compound was purified by Combi-flash using 10% MeOH in DCM to afford 1-(2-aminopyrimidin-4-yl)azetidin-3-ol (300 mg, 66.5%) as an off-white solid. LC-MS: [M+H⁺]=167.1.

### Steps 2 and 3:

Example 680 was prepared following the procedure described for example 679 by substituting 4-(2-aminopyrimidin-4-yl)cyclohexan-1-ol with 1-(2-aminopyrimidin-4-yl)azetidin-3-ol to afford the title compound as a white solid. LC-MS: [M+H⁺]=528.2. ¹H NMR (CD₃OD, 400 MHZ): δ 8.44-8.40 (m, 1H), 7.74-7.64 (m, 3H), 7.53-7.46 (m, 2H), 7.29-7.23 (m, 1H), 6.79 (bs, 2H), 6.14 (d, J=7.2 Hz, 1H), 5.08-5.02 (m, 1H), 4.76-4.75 (m, 1H), 4.51-4.46 (m, 1H), 4.37-4.34 (m, 1H), 4.06-3.93 (m, 2H), 3.83-3.77 (m, 1H), 3.44-3.38 (m, 1H), 3.24-3.21 (m, 1H), 3.02-2.97 (m, 1H), 2.81 (s, 3H), 2.69-2.67 (m, 1H), 1.98-1.88 (m, 2H), 1.59-1.52 (m, 1H).

### Synthesis of 2-fluoro-4-((4-(4-hydroxycyclohex-1-en-1-yl)pyrimidin-2-yl)amino)-N-(8-methylisoquinolin-1-yl)-N—((R)-piperidin-3-yl)benzamide (Example 681)

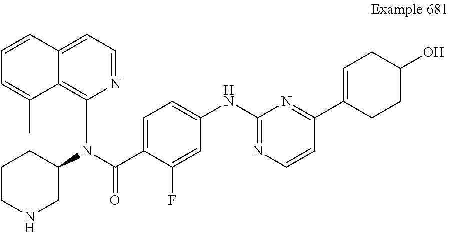 [SMILES: Cc1cccc2ccnc(N(C(=O)c3ccc(Nc4nccc(C5=CCC(O)CC5)n4)cc3F)[C@@H]3CCCNC3)c12]

Example 681 was synthesized following the procedure for Example 679 by substituting 4,4,5,5-tetramethyl-2-(1,4-dioxaspiro[4.5]dec-7-en-8-yl)-1,3,2-dioxaborolane with 4-(4,4,5,5-tetramethyl-1,3,2-dioxaborolan-2-yl)cyclohex-3-en-1-ol, provided the title compound as a white solid. LC-MS: [M+H⁺]=553.3. 1H NMR (CD₃OD, 400 MHZ): δ 8.43-8.39 (m, 1H), 8.28 (d, J=5.2 Hz, 1H), 7.73-7.64 (m, 2H), 7.58-7.42 (m, 3H), 6.94 (d. J=5.2 Hz, 1H), 6.82-6.79 (m, 2H), 6.64-6.59 (m, 1H), 5.01-4.95 (m, 1H), 3.99-3.94 (m, 2H), 3.73-3.68 (m, 1H), 3.23-3.21 (m, 1H), 2.91-2.88 (m, 1H), 2.80 (s, 3H), 2.61-2.56 (m, 3H), 2.46-2.44 (m, 1H), 2.25-2.23 (m, 1H), 1.91-1.87 (m, 1H), 1.84-1.75 (m, 2H), 1.75-1.71 (m, 1H), 1.55-1.51 (m, 1H).

### Synthesis of (R)-2-fluoro-4-((4-(1-methyl-1,2,3,6-tetrahydropyridin-4-yl)pyrimidin-2-yl)amino)-N-(8-methylisoquinolin-1-yl)-N-(piperidin-3-yl)benzamide (Example 682)

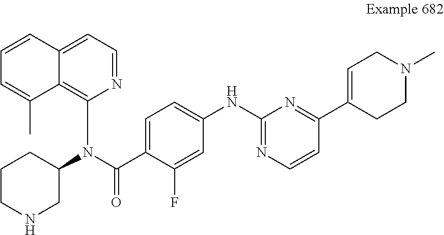 [SMILES: Cc1cccc2ccnc(N(C(=O)c3ccc(Nc4nccc(C5=CCN(C)CC5)n4)cc3F)[C@@H]3CCCNC3)c12]

Example 682 was prepared according to the procedure for Example 679 by substituting 4,4,5,5-tetramethyl-2-(1,4-dioxaspiro[4.5]dec-7-en-8-yl)-1,3,2-dioxaborolane with 1-methyl-4-(4,4,5,5-tetramethyl-1,3,2-dioxaborolan-2-yl)-1,2,3,6-tetrahydropyridine, provided the title compound as an off-white solid. LC-MS: [M+H⁺]=552.3. ¹H NMR (CD₃OD, 400 MHZ): δ 8.44-8.40 (m, 2H), 7.76-7.71 (m, 1H), 7.68-7.64 (m, 1H), 7.52-7.43 (m, 3H), 7.12 (d, J=5.2 Hz, 1H), 6.89-6.81 (m, 2H), 6.70-6.66 (m, 1H), 5.10-5.03 (m, 1H), 4.18-4.14 (m, 1H), 3.97-3.78 (m, 3H), 3.51-3.38 (m, 2H), 3.26-3.23 (m, 1H), 3.13 (s, 3H), 3.06-3.03 (m, 2H), 2.80 (s, 3H), 2.69-2.66 (m, 1H), 1.98-1.87 (m, 3H), 1.58-1.55 (m, 1H).

### Synthesis of (R)-2-fluoro-N-(8-methylisoquinolin-1-yl)-N-(piperidin-3-yl)-4-((4-(pyridin-4-ylamino)pyrimidin-2-yl)amino)benzamide (Example 683)

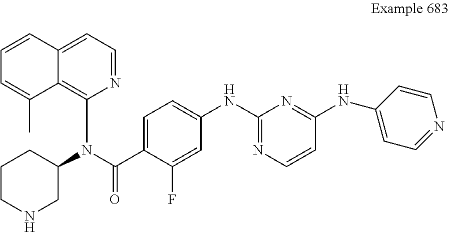 [SMILES: Cc1cccc2ccnc(N(C(=O)c3ccc(Nc4nccc(Nc5ccncc5)n4)cc3F)[C@@H]3CCCNC3)c12]

Example 683 was prepared according to the procedure described for Example 680 by substituting azetidin-3-ol with 4-aminopyridine, provided the title compound as an off-white solid. LC-MS: [M+H⁺]=549.05. ¹H NMR (CD₃OD, 400 MHz): δ 8.56-8.54 (m, 2H), 8.44-8.40 (m, 1H), 8.23-8.20 (m, 3H), 7.76-7.72 (m, 1H), 7.69-7.65 (m, 1H), 7.51-7.46 (m, 2H), 7.35-7.27 (m, 1H), 6.88-6.85 (m, 2H), 6.65 (d, J=5.6 Hz, 1H), 5.10-5.05 (m, 1H), 4.01-3.98 (m, 1H), 3.85-3.79 (m, 1H), 3.47-3.38 (m, 1H), 3.03-2.99 (m, 1H), 2.81 (s, 3H), 2.71-2.67 (m, 1H), 1.96-1.89 (m, 2H), 1.56-1.52 (m, 1H).

### Synthesis of 2-fluoro-4-((4-((R)-3-hydroxypyrrolidin-1-yl)pyrimidin-2-yl)amino)-N-(8-methylisoquinolin-1-yl)-N—((R)-piperidin-3-yl)benzamide (Example 684)

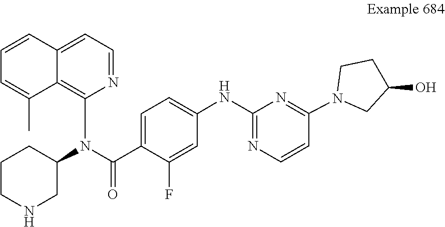 [SMILES: Cc1cccc2ccnc(N(C(=O)c3ccc(Nc4nccc(N5CC[C@@H](O)C5)n4)cc3F)[C@@H]3CCCNC3)c12]

Example 684 was prepared according to the procedure described for Example 680 by substituting azetidin-3-ol with (R)-pyrrolidin-3-ol, provided the title compound as an off-white solid. LC-MS: [M+H⁺]=542.3. ¹H NMR (CD₃OD, 400 MHZ): δ 8.44-8.40 (m, 1H), 7.75-7.72 (m, 2H), 7.68-7.63 (m, 1H), 7.51-7.45 (m, 2H), 7.31-7.24 (m, 1H), 6.81-6.78 (m, 2H), 6.37-6.31 (m, 1H), 5.09-5.03 (m, 1H), 4.59 (s, 1H), 3.99-3.96 (m, 1H), 3.71-3.67 (m, 2H), 3.59-3.58 (m, 2H), 3.41-3.36 (m, 1H), 3.01-2.99 (m, 2H), 2.85 (s, 3H), 2.71-2.69 (m, 1H), 2.23-2.15 (m, 2H), 1.94-1.88 (m, 2H), 1.59-1.53 (m, 1H).

### Synthesis of (R)-4-((4-(3,6-dihydro-2H-pyran-4-yl)pyrimidin-2-yl)amino)-2-fluoro-N-(8-methylisoquinolin-1-yl)-N-(piperidin-3-yl)benzamide (Example 685)

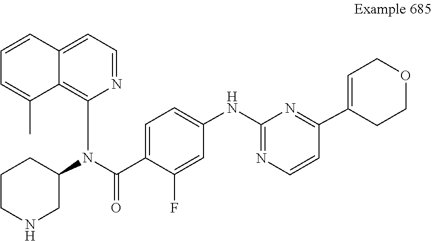 [SMILES: Cc1cccc2ccnc(N(C(=O)c3ccc(Nc4nccc(C5=CCOCC5)n4)cc3F)[C@@H]3CCCNC3)c12]

Example 685 was prepared following the procedure for example 679 by substituting 4-(2-aminopyrimidin-4-yl) cyclohexan-1-ol with 4-(3,6-dihydro-2H-pyran-4-yl)pyrimidin-2-amine to afford the title compound as an off-white solid. LC-MS: [M+H⁺]=539.3. ¹H NMR (CD₃OD, 400 MHZ): δ 8.43-8.39 (m, 1H), 8.35-8.32 (m, 1H), 7.74-7.69 (m, 1H), 7.67-7.62 (m, 1H), 7.53-7.42 (m, 3H), 6.96 (d, J=5.2 Hz, 1H), 6.89 (s, 1H), 6.84-6.83 (m, 1H), 6.63-6.59 (m, 1H), 5.07-5.01 (m, 1H), 4.35-4.34 (m, 2H), 3.91-3.89 (m, 3H), 3.81-3.78 (m, 1H), 3.47-3.38 (m, 1H), 2.96-2.95 (m, 1H), 2.79 (m, 3H), 2.66-2.65 (m, 1H), 2.51 (bs, 2H), 1.93-1.89 (m, 2H), 1.59-1.55 (m, 1H).

### Synthesis of (R)-2-fluoro-N-(8-methylisoquinolin-1-yl)-4-((4-(piperazin-1-ylmethyl)pyrimidin-2-yl)amino)-N-(piperidin-3-yl)benzamide (Example 686)

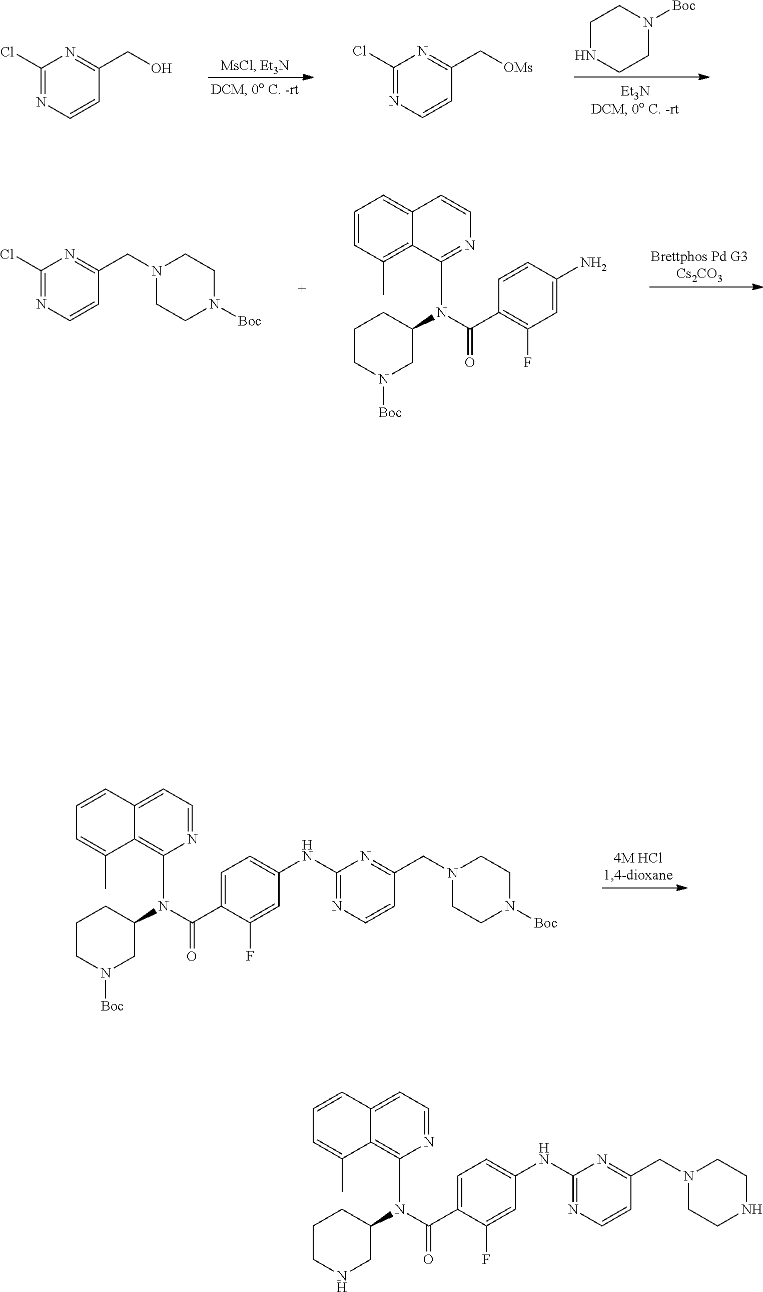

### Step 1:

To a stirred solution of (2-chloropyrimidin-4-yl) methanol (1 g, 6.92 mmol) in DCM (20 mL) was added methane sulfonyl chloride (0.647 mL, 8.30 mmol) and TEA (1.44 mL, 10.38 mmol) at 0° C. and stirred at room temperature for 2 h. The reaction mixture was poured into sat.NaHCO₃ solution (120 mL) and extracted with DCM (3×80 mL). The combined organic layer was washed with brine solution (100 mL), dried over Na₂SO₄, and concentrated under reduced pressure to afford (2-chloropyrimidin-4-yl) methyl methane sulfonate (1.2 g) as brown liquid.

### Step 2:

To a stirred solution of 2-chloro-4-((methylsulfonyl)methyl)pyrimidine (600 mg, 2.90 mmol) in DCM (10 mL) was added triethylamine (1.214 mL, 8.71 mmol) and tert-butyl piperazine-1-carboxylate (649 mg, 3.48 mmol) at 0° C. and stirred at room temperature for 16 h. The reaction mixture was poured into ice cold water (80 mL) and extracted with DCM (3×60 mL). The combined organic layer was washed with brine solution (100 mL), dried over Na₂SO₄, and concentrated under reduced pressure. The obtained compound was purified by combi-flash using 60% EtOAc in Hexane to afford tert-butyl 4-((2-chloropyrimidin-4-yl)methyl)piperazine-1-carboxylate (400 mg, 38%) as a brownish liquid.

### Step 3 and 4:

Example 686 was prepared following the procedure for example 679 by substituting 4-(2-aminopyrimidin-4-yl)cyclohexan-1-ol with tert-butyl 4-((2-chloropyrimidin-4-yl)methyl)piperazine-1-carboxylate to afford the tile compound as a yellow solid. LC-MS: [M+H⁺]=555.35. ¹H NMR (CD₃OD, 400 MHZ): δ 8.50 (d, J=5.2 Hz, 1H), 8.46-8.42 (m, 1H), 7.80-7.74 (m, 1H), 7.69-7.65 (m, 1H), 7.58-7.46 (m, 3H), 6.97-6.94 (m, 2H), 6.74-6.70 (m, 1H), 5.10-5.02 (m, 1H), 4.56 (s, 2H), 3.98-3.95 (m, 1H), 3.82-3.80 (m, 4H), 3.69-3.68 (m, 4H), 3.41-3.34 (m, 1H), 3.03-3.00 (m, 1H), 3.01-2.99 (m, 1H), 2.81 (s, 3H), 1.96-1.89 (m, 2H), 1.59-1.54 (m, 1H), 1.38-1.36 (m, 1H).

### Synthesis of (R)-2-fluoro-N-(8-methylisoquinolin-1-yl)-4-((4-(piperidin-1-ylmethyl)pyrimidin-2-yl)amino)-N-(piperidin-3-yl)benzamide (Example 687)

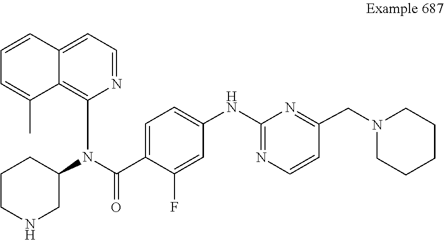 [SMILES: Cc1cccc2ccnc(N(C(=O)c3ccc(Nc4nccc(CN5CCCCC5)n4)cc3F)[C@@H]3CCCNC3)c12]

Example 687 was synthesized following the procedure for example 686 by substituting tert-butyl piperazine-1-carboxylate with piperidine to afford the title compound as a as light brown solid. LC-MS: [M+H⁺]=554.1. ¹H NMR (CD₃OD, 400 MHZ): δ 8.48-8.42 (m, 2H), 7.81 (d, J=5.6 Hz, 1H), 7.71-7.67 (m, 1H), 7.54-7.47 (m, 3H), 6.93-6.88 (m, 2H), 6.78-6.74 (m, 1H), 5.09-5.02 (m, 1H), 4.37 (s, 2H), 3.99-3.96 (m, 1H), 3.85-3.82 (m, 1H), 3.65-3.61 (m, 2H), 3.41-3.38 (m, 1H), 3.12-3.06 (m, 3H), 2.82 (s, 3H), 2.68-2.66 (m, 1H), 1.96-1.93 (m, 7H), 1.57-1.53 (m, 2H).

### Synthesis of (R)-1-(2-fluoro-4-(1-methyl-1H-1,2,3-triazol-4-yl)-N-(piperidin-3-yl)benzamido)-N-methylisoquinoline-6-carboxamide (Example 688)

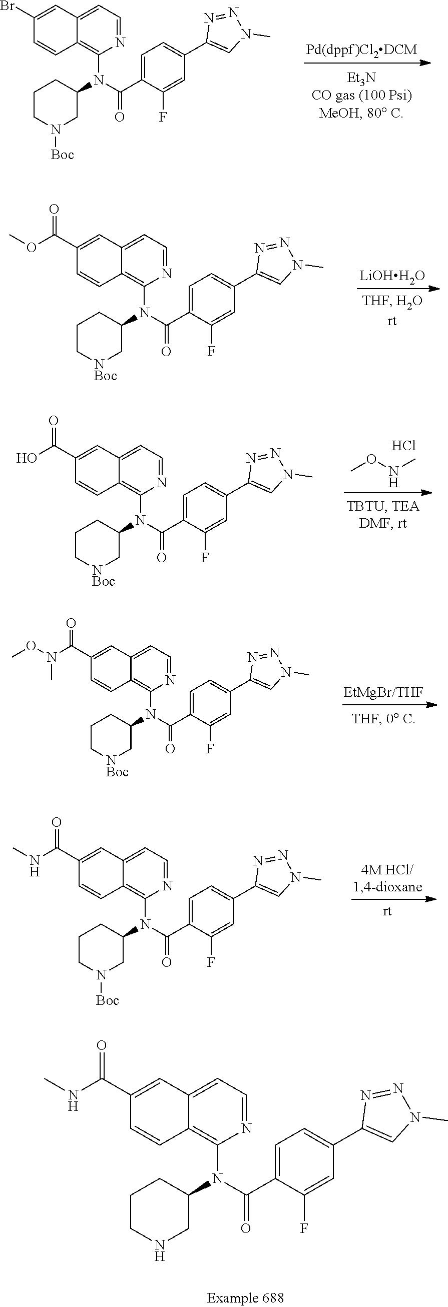

Example 688 was prepared according to the procedure described for Example 139 by substituting methyl (R)-3-(4-((1-(tert-butoxycarbonyl)piperidin-3-yl)(8-methylisoquinolin-1-yl)carbamoyl)-3-fluorophenyl)-3H-[1,2,3]triazolo[4,5-b]pyridine-5 carboxylate with tert-butyl (R)-3-(N-(6-bromoisoquinolin-1-yl)-2-fluoro-4-(1-methyl-1H-1,2,3-triazol-4-yl)benzamido)-piperidine-1-carboxylate, provided the title compound hydrochloride salt as a white solid. LC-MS: [M+H⁺]=488.2. ¹H NMR (CD₃OD, 400 MHZ): δ 8.52 (d, J=5.2 Hz, 1H), 8.24-8.17 (m, 3H), 8.04 (d, J=8.8 Hz, 1H), 7.85 (d, J=5.6 Hz, 1H), 7.26-7.23 (m, 1H), 7.19-7.15 (m, 1H), 7.10-7.06 (m, 1H), 5.29-5.23 (m, 1H), 4.06 (s, 3H), 3.95-3.92 (m, 1H), 3.72-3.67 (m, 1H), 3.31-3.30 (m, 1H), 2.89 (s, 3H), 2.86-2.81 (m, 1H), 2.01-1.97 (m, 3H), 1.32-1.21 (m, 1H).

### Synthesis of (R)-4-(4-methyl-1H-1,2,3-triazol-1-yl)-N-(3-methylthieno[3,2-c]pyridin-4-yl)-N-(piperidin-3-yl)benzamide (Example 689)

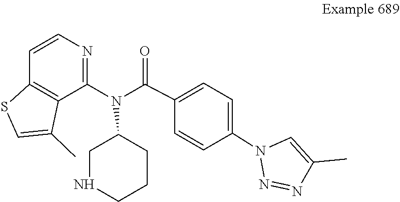 [SMILES: Cc1cn(-c2ccc(C(=O)N(c3nccc4scc(C)c34)[C@@H]3CCCNC3)cc2)nn1]

Example 689 was prepared according to the procedure described for Example 444 by substituting 2-fluoro-4-(1-methyltriazol-4-yl)benzoyl chloride and tert-butyl (3R)-3-[(6-chloro-8-methyl-1-isoquinolyl) amino] piperidine-1-carboxylate with 4-(4-methyltriazol-1-yl)benzoyl chloride and tert-butyl (3R)-3-[(3-methylthieno[3,2-c]pyridin-4-yl)amino]-piperidine-1-carboxylate, provided the title compound hydrochloride salt as a brown solid. LC-MS: [M+H]=433.2. ¹H NMR (400 MHZ, CD₃OD): δ 8.48-8.39 (m, 1H), 8.16 (s, 1H), 8.00-7.92 (m, 1H), 7.57 (d, J=8.6 Hz, 2H), 7.41-7.31 (m, 3H), 5.19-5.11 (m, 1H), 3.91 (br dd, J=2.9, 11.0 Hz, 1H), 3.78-3.69 (m, 1H), 2.97-2.88 (m, 1H), 2.57 (s, 1H), 2.49 (s, 3H), 2.33 (s, 3H), 1.96 (s, 1H), 1.95-1.86 (m, 2H), 1.84-1.76 (m, 1H), 1.35-1.25 (m, 2H).

### Synthesis of (R)-2-fluoro-4-(4-methyl-1H-1,2,3-triazol-1-yl)-N-(3-methylthieno[3,2-c]pyridin-4-yl)-N-(piperidin-3-yl)benzamide (Example 690)

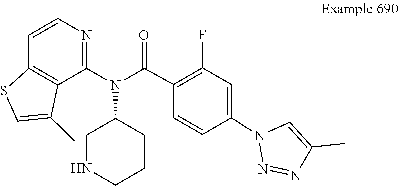 [SMILES: Cc1cn(-c2ccc(C(=O)N(c3nccc4scc(C)c34)[C@@H]3CCCNC3)c(F)c2)nn1]

Example 690 was prepared according to the procedure described for Example 444 by substituting 2-fluoro-4-(1-methyltriazol-4-yl)benzoic acid and tert-butyl (3R)-3-[(6-chloro-8-methyl-1-isoquinolyl) amino] piperidine-1-carboxylate with 2-fluoro-4-(4-methyltriazol-1-yl) benzoic acid and tert-butyl (3R)-3-[(3-methylthieno[3,2-c]pyridin-4-yl)amino]piperidine-1-carboxylate, provided the title compound hydrochloride salt as an off white solid. LC-MS: [M+H⁺]=451.3. ¹H NMR (400 MHZ, DMSO-d₆): δ ppm 9.58-8.89 (m, 2H), 8.49-8.42 (m, 1H), 8.40-8.32 (m, 1H), 8.11-7.95 (m, 1H), 7.69-7.56 (m, 2H), 7.47-7.34 (m, 1H), 7.19-7.10 (m, 1H), 4.99-4.91 (m, 1H), 3.74 (br d, J=10.5 Hz, 1H), 3.68-3.39 (m, 1H), 3.32-3.17 (m, 2H), 2.79-2.65 (m, 1H), 2.58-2.52 (m, 3H), 2.25 (s, 3H), 1.99-1.87 (m, 1H), 1.68 (br s, 4H), 1.32-1.00 (m, 2H).

### Synthesis of (R)—N-(6-chloro-8-methylisoquinolin-1-yl)-2-fluoro-4-(4-methyl-1H-1,2,3-triazol-1-yl)-N-(piperidin-3-yl)benzamide (Example 691)

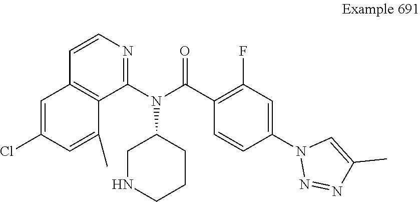 [SMILES: Cc1cn(-c2ccc(C(=O)N(c3nccc4cc(Cl)cc(C)c34)[C@@H]3CCCNC3)c(F)c2)nn1]

Example 691 was prepared according to the procedure described for Example 236 by substituting methyl 4-aminobenzoate and tert-butyl (R)-3-((8-methylisoquinolin-1-yl)amino)piperidine-1-carboxylate with methyl 4-amino-2-fluorobenzoate and tert-butyl (R)-3-((6-chloro-8-methylisoquinolin-1-yl)amino)piperidine-1-carboxylate, provided the title compound hydrochloride salt as a white solid. LC-MS: [M+H⁺]=479.3. ¹H NMR (400 MHZ, CD₃OD): δ ppm 8.50-8.38 (m, 1H), 8.14 (s, 1H), 7.76-7.64 (m, 2H), 7.54-7.36 (m, 2H), 7.29 (br d, J=8.5 Hz, 1H), 7.04 (t, J=7.9 Hz, 1H), 5.15-4.99 (m, 1H), 4.00 (br d, J=10.0 Hz, 1H), 3.86-3.74 (m, 1H), 3.50-3.36 (m, 1H), 3.09-2.94 (m, 1H), 2.89-2.78 (m, 3H), 2.32 (s, 3H), 2.07-1.83 (m, 3H), 1.66-1.51 (m, 1H).

### Synthesis of (R)-2-fluoro-4-(1-methyl-1H-1,2,3-triazol-4-yl)-N-(piperidin-3-yl)-N-(6-(prop-1-en-2-yl)isoquinolin-1-yl)benzamide (Example 692)

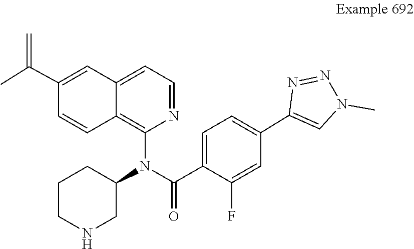 [SMILES: C=C(C)c1ccc2c(N(C(=O)c3ccc(-c4cn(C)nn4)cc3F)[C@@H]3CCCNC3)nccc2c1]

Example 692 was prepared according to the procedure described for Example 97 by substituting tert-butyl (R)-3-(4-(3H-[1,2,3]triazolo[4,5-b]pyridin-3-yl)-N-(6-bromoisoquinolin-1-yl)-2-fluorobenzamido) piperidine-1-carboxylate and 756 with tert-butyl (R)-3-(N-(6-bromoisoquinolin-1-yl)-2-fluoro-4-(1-methyl-1H-1,2,3-triazol-4-yl) benzamido) piperidine-1-carboxylate and 4,4,5,5-tetramethyl-2-(prop-1-en-2-yl)-1,3,2-dioxaborolane, provided the title compound hydrochloride salt as an off-white solid. LC-MS: [M+H⁺]=471.2. ¹H NMR (CD₃OD, 400 MHZ): δ 8.44-8.32 (m, 1H), 8.17-8.14 (m, 1H), 8.10-8.03 (m, 1H), 7.96-7.90 (m, 1H), 7.87-7.74 (m, 1H), 7.69-7.64 (m, 1H), 7.28-7.18 (m, 2H), 7.11-7.06 (m, 1H), 6.60-6.54 (m, 1H), 5.66-5.63 (m, 1H), 5.34-5.32 (m, 1H), 5.26-5.22 (m, 1H), 4.05 (s, 3H), 3.93-3.90 (m, 1H), 3.69-3.63 (m, 1H), 2.88-2.81 (m, 1H), 2.21-2.19 (m, 2H), 2.02-1.88 (m, 5H), 1.32-1.19 (m, 1H).

### Synthesis of (R)-2-fluoro-N-(8-methylisoquinolin-1-yl)-N-(piperidin-3-yl)-4-((4-((thiazol-2-ylmethyl)amino)pyrimidin-2-yl)amino)benzamide (Example 693)

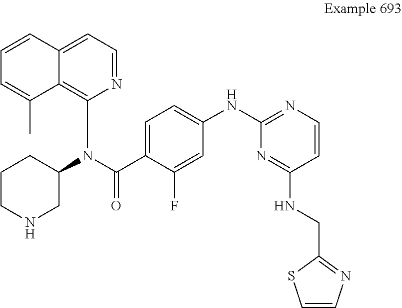 [SMILES: Cc1cccc2ccnc(N(C(=O)c3ccc(Nc4nccc(NCc5nccs5)n4)cc3F)[C@@H]3CCCNC3)c12]

Example 693 was synthesized following the procedure for example 684 by substituting (R)-pyrrolidin-3-ol with thiazol-2-ylmethanamine to afford the title compound as a light brown solid. LC-MS: [M+H⁺]=568.95. ¹H NMR (CD₃OD, 400 MHZ): δ 8.42-8.38 (m, 1H), 7.81 (d, J=5.6 Hz, 1H), 7.75-7.63 (m, 3H), 7.50-7.41 (m, 3H), 7.25-7.21 (m, 1H), 6.70 (d, J=8.8 Hz, 1H), 6.54-6.50 (m, 1H), 6.07 (d, J=6.0 Hz, 1H), 5.08-5.04 (m, 1H), 4.80 (s, 2H), 3.94-3.91 (m, 1H), 3.82-3.76 (m, 1H), 3.39-3.36 (m, 1H), 3.02-2.97 (m, 1H), 2.79 (s, 3H), 1.93-1.85 (m, 3H), 1.58-1.53 (m, 1H).

### Synthesis of (R)-2-fluoro-4-((4-(((1-methyl-1H-pyrazol-4-yl)methyl)amino)pyrimidin-2-yl)amino)-N-(8-methylisoquinolin-1-yl)-N-(piperidin-3-yl)benzamide (Example 694)

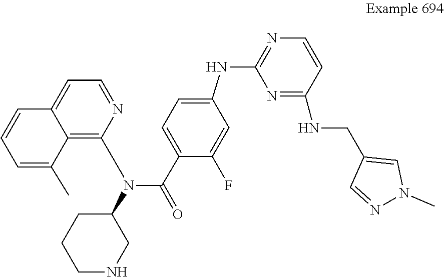 [SMILES: Cc1cccc2ccnc(N(C(=O)c3ccc(Nc4nccc(NCc5cnn(C)c5)n4)cc3F)[C@@H]3CCCNC3)c12]

Example 694 was synthesized following the procedure for example 680 by substituting azetidin-3-ol hydrochloride with (1-methyl-1H-pyrazol-4-yl)methenamine to afford the title compound as a as light-brown solid. LC-MS: [M+H⁺]=566.3. ¹H NMR (CD₃OD, 400 MHZ): δ 8.40-8.36 (m, 1H), 7.68-7.60 (m, 4H), 7.50-7.43 (m, 3H), 7.20-7.13 (m, 1H), 6.84-6.78 (m, 2H), 6.22 (d, J=7.2 Hz, 1H), 5.07-5.02 (m, 1H), 4.39 (s, 2H), 3.90 (s, 3H), 3.81-3.76 (m, 1H), 3.43-3.37 (m, 1H), 3.23-3.17 (m, 1H), 3.01-2.98 (m, 1H), 2.81 (s, 3H), 2.69-2.67 (m, 1H), 1.98-1.87 (m, 2H), 1.54-1.50 (m, 1H).

### Synthesis of (R)-2-fluoro-N-(8-methylisoquinolin-1-yl)-N-(piperidin-3-yl)-4-((4-((pyridin-2-ylmethyl)amino)pyrimidin-2-yl)amino)benzamide (Example 695)

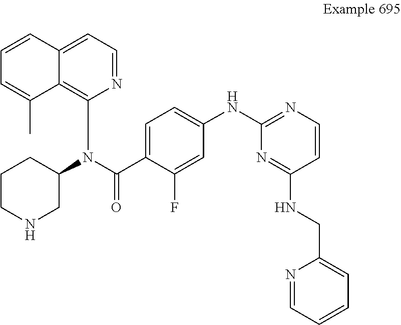 [SMILES: Cc1cccc2ccnc(N(C(=O)c3ccc(Nc4nccc(NCc5ccccn5)n4)cc3F)[C@@H]3CCCNC3)c12]

Example 695 was synthesized following the procedure for Example 684 by substituting (R)-pyrrolidin-3-ol with pyridin-2-ylmethanamine to afford the title product as a pale-brown solid. LC-MS: [M+H⁺]=563.3. ¹H NMR (CD₃OD, 400 MHZ): δ 8.76 (d, J=4.8 Hz, 1H), 8.50-8.47 (m, 1H), 8.42-8.39 (m, 1H), 7.96-7.92 (m, 1H), 7.87 (d, J=8.0 Hz, 1H), 7.81 (d, J=6.0 Hz, 1H), 7.72-7.69 (m, 1H), 7.66-7.62 (m, 1H), 7.52-7.44 (m, 2H), 6.84-6.74 (m, 2H), 6.57 (d, J=8.4 Hz, 1H), 6.50 (d, J=7.2 Hz, 1H), 5.10-5.02 (m, 1H), 4.94 (s, 2H), 3.82-3.76 (m, 1H), 3.52-3.38 (m, 2H), 3.20-3.12 (m, 1H), 2.81 (s, 3H), 2.71-2.69 (m, 1H), 1.99-1.87 (m, 2H), 1.52-1.48 (m, 1H).

### Synthesis of (R)-2-fluoro-N-(8-methylisoquinolin-1-yl)-N-(piperidin-3-yl)-4-((4-((pyridin-3-ylmethyl)amino)pyrimidin-2-yl)amino)benzamide (Example 696)

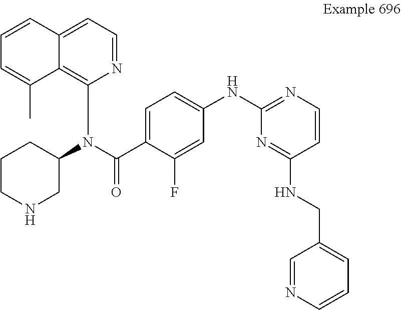 [SMILES: Cc1cccc2ccnc(N(C(=O)c3ccc(Nc4nccc(NCc5cccnc5)n4)cc3F)[C@@H]3CCCNC3)c12]

Example 696 was synthesized following the procedure for Example 684 by substituting (R)-pyrrolidin-3-ol with pyridin-3-ylmethanamine to afford the title product as an off-white solid. LC-MS: [M+H⁺]=563.3. ¹H NMR (CD₃OD, 400 MHZ): δ 8.84 (d, J=5.2 Hz, 1H), 8.79 (s, 1H), 8.50 (d, J=8.0 Hz, 1H), 8.43-8.39 (m, 1H), 8.09-8.05 (m, 1H), 7.77-7.70 (m, 2H), 7.66-7.64 (m, 1H), 7.52-7.44 (m, 2H), 6.99-6.92 (m, 1H), 6.83-6.79 (m, 1H), 6.66 (d, J=8.4 Hz, 1H), 6.41 (d, J=7.2 Hz, 1H), 5.08-5.02 (m, 1H), 4.80 (s, 2H), 3.81-3.76 (m, 1H), 3.51-3.37 (m, 2H), 3.05-2.94 (m, 1H), 2.81 (s, 3H), 2.69-2.64 (m, 1H), 1.93-1.87 (m, 2H), 1.55-1.48 (m, 1H).

### Synthesis of (R)-2-fluoro-N-(8-methylisoquinolin-1-yl)-N-(piperidin-3-yl)-4-((4-((pyridin-4-ylmethyl)amino)pyrimidin-2-yl)amino)benzamide (Example 697)

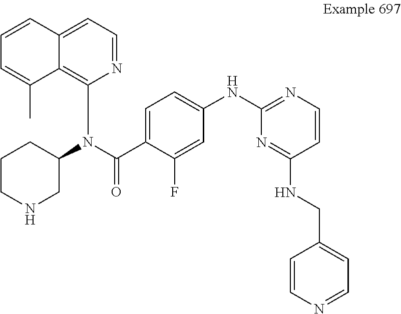 [SMILES: Cc1cccc2ccnc(N(C(=O)c3ccc(Nc4nccc(NCc5ccncc5)n4)cc3F)[C@@H]3CCCNC3)c12]

Example 697 was synthesized following the procedure for Example 684 by substituting (R)-pyrrolidin-3-ol with pyridin-4-ylmethanamine to afford the title product as pale-yellow solid. LC-MS: [M+H⁺]=563.35. ¹H NMR (CD₃OD, 400 MHZ): δ 8.81-8.80 (m, 2H), 8.43-8.40 (m, 1H), 9.93-7.92 (m, 2H), 7.79 (d, J=7.2 Hz, 1H), 7.74-7.72 (m, 1H), 7.66-7.74 (m, 1H), 7.53-7.45 (m, 2H), 6.85-6.71 (m, 2H), 6.56 (d, J=8.4 Hz, 1H), 6.47 (d, J=7.2 Hz, 1H), 5.07-5.03 (m, 1H), 3.82-3.76 (m, 1H), 3.47-3.38 (m, 2H), 3.20-3.12 (m, 1H), 3.01-2.98 (m, 1H), 2.80 (s, 3H), 2.69-2.66 (m, 1H), 1.96-1.87 (m, 2H), 1.59-1.53 (m, 1H), 0.913-0.872 (m, 1H).

### Synthesis of (R)-4-(5-ethyl-1,3,4-thiadiazol-2-yl)-2-fluoro-N-(3-methylthieno[3,2-c]pyridin-4-yl)-N-(piperidin-3-yl)benzamide (Example 698)

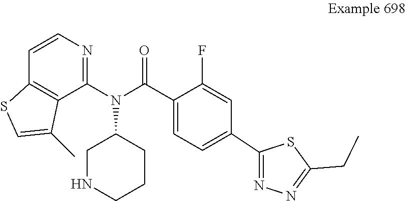 [SMILES: CCc1nnc(-c2ccc(C(=O)N(c3nccc4scc(C)c34)[C@@H]3CCCNC3)c(F)c2)s1]

Example 698 was prepared according to the procedure described for Example 127 by substituting tert-butyl (R)-3-((8-methylisoquinolin-1-yl)amino)piperidine-1-carboxylate with tert-butyl (R)-3-((3-methylthieno[3,2-c]pyridin-4-yl)amino)piperidine-1-carboxylate and 2-bromo-5-methyl-1,3,4-thiadiazole with 2-bromo-5-ethyl-1,3,4-thiadiazole, provided the title compound as an off-white solid. LC-MS: [M+H]=482.2. ¹H NMR (400 MHz, DMSO-d₆): δ=8.83-9.12 (m, 2H), 8.53-8.79 (m, 1H), 8.19-8.47 (m, 1H), 8.05 (d, J=5.50 Hz, 1H), 7.55-7.72 (m, 2H), 7.38-7.47 (m, 1H), 7.06-7.17 (m, 1H), 4.61-5.00 (m, 1H), 3.73-3.81 (m, 1H), 3.46 (br d, J=10.88 Hz, 1H), 3.15-3.35 (m, 2H), 3.09 (q, J=7.50 Hz, 2H), 2.69-2.86 (m, 2H), 2.56-2.59 (m, 1H), 2.53 (br s, 3H), 1.88-2.01 (m, 1H), 1.76 (br d, J=9.38 Hz, 3H), 1.29 (t, J=7.50 Hz, 4H), 0.96-1.11 (m, 1H).

### Synthesis of (R)-2-fluoro-4-(5-methyl-1,3,4-oxadiazol-2-yl)-N-(3-methylthieno[3,2-c]pyridin-4-yl)-N-(piperidin-3-yl)benzamide (Example 699)

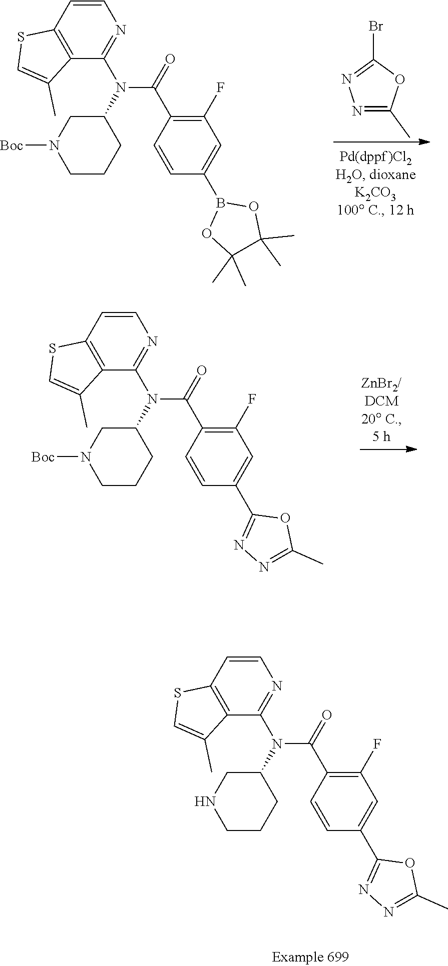

Example 699 was prepared according to the procedure described for Example 816 by substituting tert-butyl (R)-3-(4-bromo-2-chloro-N-(6-chloro-8-methylisoquinolin-1-yl)benzamido)piperidine-1-carboxylate and 4-bromo-1-methyl-triazole with tert-butyl (3R)-3-[(2-fluoro-4-iodo-benzoyl)-(3-methylthieno[3,2-c]pyridin-4-yl)amino]piperidine-1-carboxylate and 2-bromo-5-methyl-1,3,4-oxadiazole, provided the title compound as a white solid. LC-MS: [M+H]=452.1. ¹H NMR (400 MHZ, CD₃OD): δ ppm 8.37-8.27 (m, 1H), 7.86 (d, J=5.5 Hz, 1H), 7.57-7.38 (m, 3H), 7.29-7.17 (m, 1H), 4.79-4.64 (m, 1H), 3.26 (br d, J=11.8 Hz, 1H), 3.03-2.93 (m, 1H), 2.74-2.61 (m, 4H), 2.58-2.45 (m, 4H), 1.82-1.62 (m, 3H), 1.23-1.03 (m, 1H).

### Synthesis of (R)—N-(6-chloro-8-methylisoquinolin-1-yl)-4-(5-ethyl-1,3,4-thiadiazol-2-yl)-2-fluoro-N-(piperidin-3-yl)benzamide (Example 700)

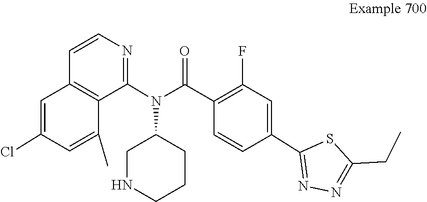 [SMILES: CCc1nnc(-c2ccc(C(=O)N(c3nccc4cc(Cl)cc(C)c34)[C@@H]3CCCNC3)c(F)c2)s1]

Example 700 was prepared according to the procedure described for Example 816 by substituting 4-bromo-2-chloro-benzoyl chloride and 4-bromo-1-methyl-triazole with 4-bromo-2-fluoro-benzoyl chloride and 2-bromo-5-ethyl-1,3,4-thiadiazole, provided the title compound hydrochloride salt as an off-white solid. LC-MS: [M+H⁺]=510.3. ¹H NMR (400 MHz, CD₃OD): δ ppm 8.48-8.42 (m, 1H), 7.76-7.64 (m, 2H), 7.52-7.39 (m, 2H), 7.32 (dd, J=1.5, 8.0 Hz, 1H), 7.05-6.96 (m, 1H), 5.11-5.02 (m, 1H), 4.03-3.96 (m, 1H), 3.86-3.76 (m, 1H), 3.46-3.37 (m, 1H), 3.18-3.09 (m, 2H), 3.06-2.96 (m, 1H), 2.90-2.81 (m, 3H), 2.02-1.85 (m, 3H), 1.68-1.49 (m, 1H), 1.39 (t, J=7.5 Hz, 3H).

### Synthesis of (R)—N-(6,8-dimethylisoquinolin-1-yl)-5-(5-methyl-1,3,4-thiadiazol-2-yl)-N-(piperidin-3-yl)picolinamide (Example 701)

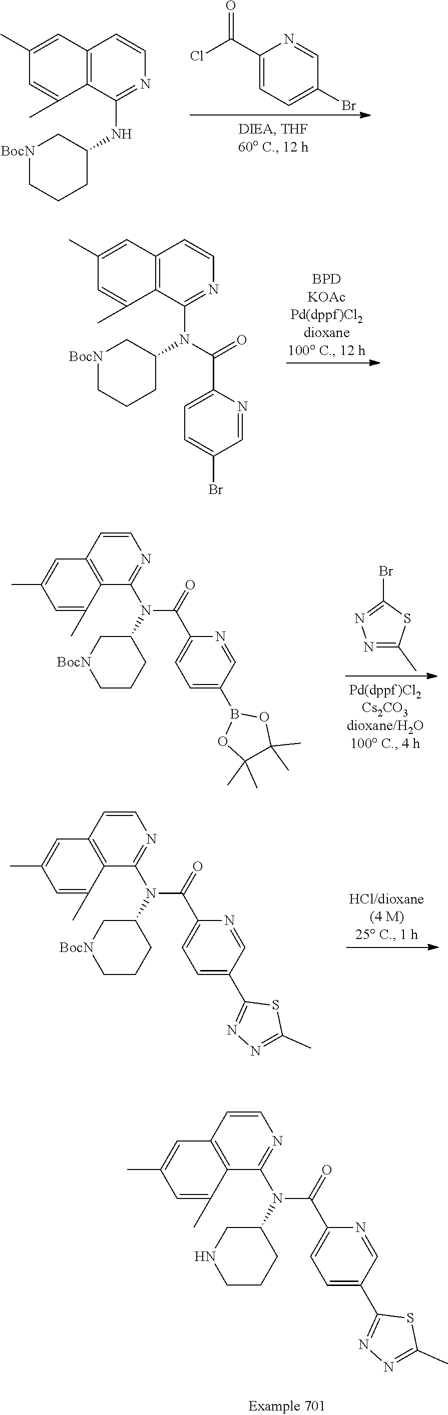

### Step 1:

A mixture of tert-butyl (R)-3-((6,8-dimethylisoquinolin-1-yl)amino)piperidine-1-carboxylate (380 mg, 1.07 mmol), 5-bromopyridine-2-carbonyl chloride (706.99 mg, 3.21 mmol), DIEA (690.80 mg, 5.34 mmol, 931.00 μL) in THF (5 mL) was degassed and purged with N₂ for 3 times at 20° C., and then the mixture was heated to 60° C. and stirred for 12 h under N₂ atmosphere. The reaction mixture was filtered and concentrated. The residue was purified by column chromatography (SiO₂, Petroleum ether/EtOAc=10/1 to 4/1) to give tert-butyl (R)-3-(5-bromo-N-(6,8-dimethylisoquinolin-1-yl)picolinamido) piperidine-1-carboxylate (766 mg, 86%) as a yellow solid. LC-MS: [M+H⁺]=539.3.

### Steps 2-4:

Example 701 was prepared according to the procedure described for Example 816 by substituting tert-butyl (R)-3-(4-bromo-2-chloro-N-(6-chloro-8-methylisoquinolin-1-yl)benzamido)piperidine-1-carboxylate and 4-bromo-1-methyl-triazole with (R)-3-(5-bromo-N-(6,8-dimethylisoquinolin-1-yl)picolinamido) piperidine-1-carboxylate and 2-bromo-5-methyl-1,3,4-thiadiazole, provided the title compound hydrochloride salt as a white solid. LC-MS: [M+H⁺]=459.3. ¹H NMR (400 MHz, CD₃OD): δ ppm 8.56-8.26 (m, 3H), 8.26-7.95 (m, 2H), 7.91-7.70 (m, 1H), 7.64-7.37 (m, 1H), 5.15-4.94 (m, 1H), 4.05 (br t, J=9.7 Hz, 1H), 3.60-3.43 (m, 1H), 3.38-3.33 (m, 1H), 2.98-2.87 (m, 1H), 2.78 (d, J=0.9 Hz, 3H), 2.77-2.67 (m, 3H), 2.60-2.49 (m, 3H), 1.96-1.65 (m, 3H), 1.16-0.91 (m, 1H).

### Synthesis of (R)—N-(6,8-dimethylisoquinolin-1-yl)-2-fluoro-4-(1-methyl-1H-1,2,3-triazol-4-yl)-N-(piperidin-3-yl)benzamide (Example 702)

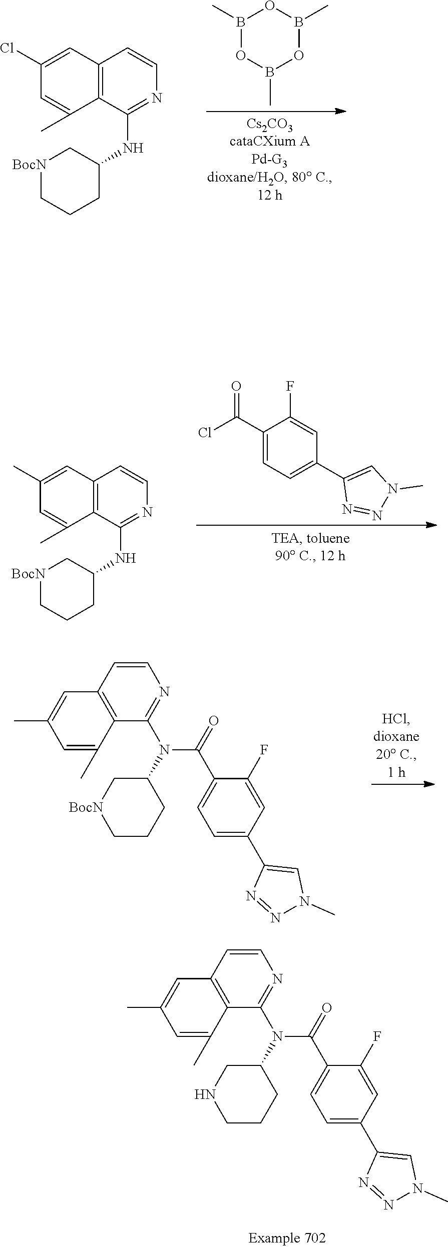

### Step 1:

A mixture of tert-butyl (3R)-3-[(6-chloro-8-methyl-1-isoquinolyl)amino]piperidine-1-carboxylate (500 mg, 1.33 mmol), 2,4,6-trimethyl-1,3,5,2,4,6-trioxatriborinane (333.96 mg, 2.66 mmol), [2-(2-aminophenyl) phenyl] palladium (1+); bis(1-adamantyl)-butyl-phosphane; methanesulfonate (96.87 mg, 133.02 μmol), Cs₂CO₃ (1.30 g, 3.99 mmol) in dioxane (10 mL) and H₂O (2 mL) was degassed and purged with N₂ for 3 times at 20° C., and then the mixture was heated to 80° C. and stirred for 12 h under N₂ atmosphere. The reaction mixture was concentrated under reduced pressure to give a residue. The residue was purified by column chromatography to give tert-butyl (R)-3-((6,8-dimethylisoquinolin-1-yl)amino)piperidine-1-carboxylate (450 mg, 95.1%) as a brown solid. LC-MS: [M+H⁺]=356.2.

### Steps 2-3:

Example 702 was prepared according to the procedure described for Example 444 by substituting tert-butyl (3R)-3-[(6-chloro-1-isoquinolyl)amino]piperidine-1-carboxylate with tert-butyl (R)-3-((6,8-dimethylisoquinolin-1-yl)amino)piperidine-1-carboxylate, provided the title compound hydrochloride salt as a white solid. LC-MS: [M+H⁺]=459.3. ¹H NMR (400 MHz, CD₃OD): δ ppm 8.43-8.35 (m, 1H), 8.24-8.18 (m, 1H), 7.71 (d, J=5.6 Hz, 1H), 7.51-7.43 (m, 1H), 7.42-7.35 (m, 1H), 7.32-7.25 (m, 1H), 7.17 (br d, J=8.1 Hz, 1H), 7.00-6.90 (m, 1H), 5.09-4.98 (m, 1H), 4.08 (s, 3H), 4.02 (br d, J=11.8 Hz, 1H), 3.88-3.77 (m, 1H), 3.40 (br d, J=12.4 Hz, 1H), 3.06-2.95 (m, 1H), 2.87-2.78 (m, 3H), 2.44-2.35 (m, 3H), 2.01-1.84 (m, 3H), 1.60-1.47 (m, 1H).

### Synthesis of (R)—N-(6,8-dimethylisoquinolin-1-yl)-5-(1-methyl-1H-1,2,3-triazol-4-yl)-N-(piperidin-3-yl)picolinamide (Example 703)

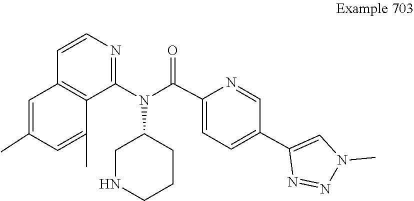

Example 703 was prepared according to the procedure described for Example 816 by substituting 4-bromo-2-chloro-benzoyl chloride and tert-butyl (3R)-3-[(6-chloro-8-methyl-1-isoquinolyl) amino] piperidine-1-carboxylate with 5-bromopyridine-2-carbonyl chloride and tert-butyl (3R)-3-[(6,8-dimethyl-1-isoquinolyl) amino] piperidine-1-carboxylate, provided the title compound hydrochloride salt as a white solid. LC-MS: [M+H⁺]=442.3. ¹H NMR (400 MHz, CD₃OD): δ ppm 8.34-8.20 (m, 3H), 8.13 (dd, J=1.9, 8.2 Hz, 1H), 7.91-7.72 (m, 2H), 7.63 (s, 1H), 7.37 (s, 1H), 5.06-4.92 (m, 1H), 4.09 (s, 3H), 3.99 (br d, J=10.8 Hz, 1H), 3.53 (br t, J=11.4 Hz, 1H), 3.34 (br s, 1H), 2.93-2.81 (m, 1H), 2.75 (s, 3H), 2.53-2.38 (m, 3H), 1.94-1.70 (m, 3H), 1.19-0.96 (m, 1H).

### Synthesis of (R)-4-(1-ethyl-1H-1,2,3-triazol-4-yl)-2-fluoro-N-(3-methylthieno[3,2-c]pyridin-4-yl)-N-(piperidin-3-yl)benzamide (Example 704)

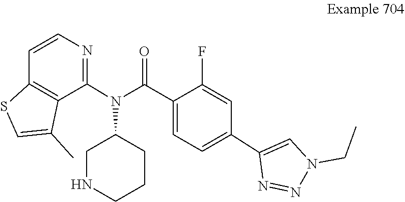 [SMILES: CCn1cc(-c2ccc(C(=O)N(c3nccc4scc(C)c34)[C@@H]3CCCNC3)c(F)c2)nn1]

Example 704 was prepared according to the procedure described for Example 127 by substituting tert-butyl (R)-3-((8-methylisoquinolin-1-yl)amino)piperidine-1-carboxylate with tert-butyl (R)-3-((3-methylthieno[3,2-c]pyridin-4-yl)amino)piperidine-1-carboxylate and 2-bromo-5-methyl-1,3,4-thiadiazole with 4-bromo-1-ethyl-1H-1,2,3-triazole, provided the title compound as an off-white solid. LC-MS: [M+H]=465.3. ¹H NMR (400 MHZ, DMSO-d₆): δ ppm 8.74-8.98 (m, 2H), 8.55 (s, 1H), 8.17-8.44 (m, 1H), 7.85-8.06 (m, 1H), 7.54-7.75 (m, 1H), 7.37-7.47 (m, 1H), 7.21-7.32 (m, 1H), 6.93-7.04 (m, 1H), 4.85-5.02 (m, 1H), 4.36 (q, J=7.38 Hz, 2H), 3.77 (dt, J=9.32, 1.78 Hz, 1H), 3.25 (br s, 2H), 2.70-2.85 (m, 1H), 1.66-1.83 (m, 3H), 1.35-1.56 (m, 4H), 1.23 (s, 1H), 0.78-1.15 (m, 2H).

### Synthesis of 2-methyl-4-(1-methyl-1H-1,2,3-triazol-4-yl)-N-(8-methylisoquinolin-1-yl)-N-(piperidin-3-yl)benzamide (Example 705)

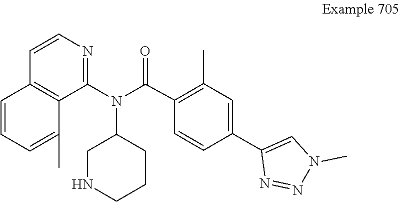 [SMILES: Cc1cc(-c2cn(C)nn2)ccc1C(=O)N(c1nccc2cccc(C)c12)C1CCCNC1]

Example 705 was synthesized following the procedure for example 111 by substituting 4-(methoxycarbonyl)phenyl) boronic acid with 3-methyl-4-(methoxycarbonyl)phenyl)boronic acid to afford the title compound as a yellow solid. LC-MS: [M+H⁺]=441.3. ¹H NMR (400 MHZ, DMSO-d₆): δ ppm 9.48-9.35 (m, 1H), 9.32-9.09 (m, 1H), 8.41-8.26 (m, 2H), 7.76-7.67 (m, 2H), 7.65-7.52 (m, 2H), 7.37 (s, 1H), 7.06 (dd, J=1.4, 8.0 Hz, 1H), 6.75 (d, J=8.1 Hz, 1H), 4.95-4.81 (m, 1H), 3.98 (s, 3H), 3.88 (br d, J=10.3 Hz, 1H), 3.47 (br d, J=10.6 Hz, 1H), 3.23 (br d, J=12.6 Hz, 1H), 2.86 (s, 3H), 2.83-2.66 (m, 1H), 2.33 (s, 3H), 1.88-1.70 (m, 3H), 1.25 (br s, 1H).

### Synthesis of (R)—N-(6-chloro-8-methylisoquinolin-1-yl)-3-methoxy-4-(5-methyl-1,3,4-thiadiazol-2-yl)-N-(piperidin-3-yl)benzamide (Example 706)

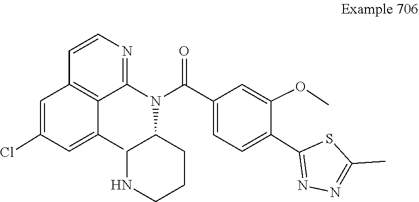 [SMILES: COc1cc(C(=O)N(c2nccc3cc(Cl)cc(C)c23)[C@@H]2CCCNC2)ccc1-c1nnc(C)s1]

Example 706 was prepared according to the procedure described for Example 816 by substituting 4-bromo-2-chloro-benzoyl chloride and 4-bromo-1-methyl-triazole with 4-bromo-3-methyoxy-benzoyl chloride and 2-bromo-5-methyl-1,3,4-thiadiazole, provided the title compound hydrochloride salt as a yellow solid. LC-MS: [M+H⁺]=508.3. ¹H NMR (400 MHz, DMSO-d₆): δ ppm 9.28-8.91 (m, 2H), 8.60 (d, J=5.5 Hz, 1H), 7.97-7.92 (m, 2H), 7.89 (d, J=5.6 Hz, 1H), 7.51 (s, 1H), 6.86 (dd, J=1.1, 8.3 Hz, 1H), 6.70 (s, 1H), 5.02-4.90 (m, 1H), 3.84-3.73 (m, 2H), 3.67 (s, 3H), 3.62-3.48 (m, 1H), 3.32-3.20 (m, 1H), 2.84-2.77 (m, 1H), 2.70 (d, J=6.4 Hz, 6H), 1.89-1.76 (m, 2H), 1.74-1.65 (m, 1H), 1.32-1.17 (m, 1H).

### Synthesis of (R)—N-(6-chloro-8-methylisoquinolin-1-yl)-3-methyl-4-(5-methyl-1,3,4-thiadiazol-2-yl)-N-(piperidin-3-yl)benzamide (Example 707)

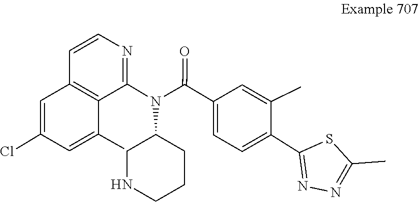 [SMILES: Cc1nnc(-c2ccc(C(=O)N(c3nccc4cc(Cl)cc(C)c34)[C@@H]3CCCNC3)cc2C)s1]

Example 707 was prepared according to the procedure described for Example 816 by substituting 4-bromo-2-chloro-benzoyl chloride and 4-bromo-1-methyl-triazole with 4-bromo-3-methyl-benzoyl chloride and 2-bromo-5-methyl-1,3,4-thiadiazole, provided the title compound hydrochloride salt as a yellow solid. LC-MS: [M+H⁺]=492.3. ¹H NMR (400 MHz, CD₃OD): δ ppm 8.54 (d, J=5.5 Hz, 1H), 7.81-7.73 (m, 2H), 7.39 (d, J=1.1 Hz, 1H), 7.29 (d, J=8.0 Hz, 1H), 7.04-6.95 (m, 2H), 5.14-5.04 (m, 1H), 3.99-3.92 (m, 1H), 3.86-3.77 (m, 1H), 3.51-3.33 (m, 2H), 3.04-2.94 (m, 1H), 2.79-2.72 (m, 6H), 2.22 (s, 3H), 2.03-1.82 (m, 3H), 1.59-1.46 (m, 1H).

### Synthesis of (R)—N-(6-chloro-8-methylisoquinolin-1-yl)-3-fluoro-4-(5-methyl-1,3,4-thiadiazol-2-yl)-N-(piperidin-3-yl)benzamide (Example 708)

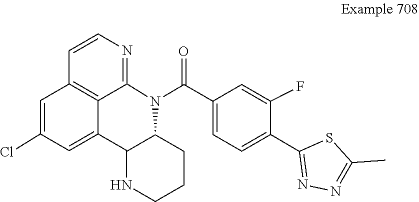 [SMILES: Cc1nnc(-c2ccc(C(=O)N(c3nccc4cc(Cl)cc(C)c34)[C@@H]3CCCNC3)cc2F)s1]

Example 708 was prepared according to the procedure described for Example 816 by substituting 4-bromo-2-chloro-benzoyl chloride and 4-bromo-1-methyl-triazole with 4-bromo-3-fluoro-benzoyl chloride and 2-bromo-5-methyl-1,3,4-thiadiazole, provided the title compound hydrochloride salt as a yellow solid. LC-MS: [M+H⁺]=496.3. ¹H NMR (400 MHz, DMSO-d₆): δ ppm 8.66-8.50 (m, 1H), 8.00-7.77 (m, 3H), 7.57-7.46 (m, 1H), 7.12-6.92 (m, 2H), 4.51 (br d, J=1.0 Hz, 1H), 3.52-3.42 (m, 1H), 3.09 (br t, J=10.7 Hz, 1H), 2.84 (br d, J=10.8 Hz, 1H), 2.75-2.66 (m, 6H), 2.29 (br d, J=2.5 Hz, 1H), 1.74-1.44 (m, 3H), 1.15-0.99 (m, 1H).

### Synthesis of (R)—N-(6-chloro-8-methylisoquinolin-1-yl)-6-(5-methyl-1,3,4-thiadiazol-2-yl)-N-(piperidin-3-yl)nicotinamide (Example 709)

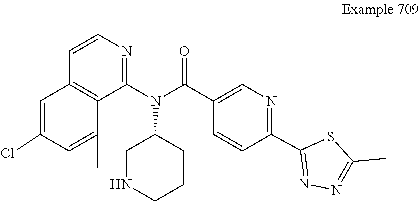 [SMILES: Cc1nnc(-c2ccc(C(=O)N(c3nccc4cc(Cl)cc(C)c34)[C@@H]3CCCNC3)cn2)s1]

Example 709 was prepared according to the procedure described for Example 816 by substituting 4-bromo-2-chloro-benzoyl chloride and 2-bromo-5-methyl-1, 3, 4-thiadiazole with 6-Bromo-3-pyridinecarbonyl chloride and 2-bromo-5-methyl-1, 3, 4-thiadiazole, provided the title compound hydrochloride salt as a white solid. LC-MS: [M+H⁺]=479.3. ¹H NMR (400 MHZ, CD₃OD): δ ppm 8.62-8.49 (m, 1H), 8.23 (s, 1H), 7.88 (br d, J=8.3 Hz, 1H), 7.84-7.79 (m, 1H), 7.79-7.76 (m, 1H), 7.60-7.53 (m, 1H), 7.44 (s, 1H), 5.31-4.99 (m, 1H), 3.98 (br d, J=11.3 Hz, 1H), 3.83-3.72 (m, 1H), 3.70-3.56 (m, 1H), 3.40 (br d, J=12.4 Hz, 1H), 3.07-2.92 (m, 1H), 2.83-2.71 (m, 6H), 2.21-1.82 (m, 3H), 1.57-1.39 (m, 1H).

### 2-fluoro-N-(6-fluoro-8-methyl-1-isoquinolyl)-4-(5-methoxy-1,3,4-thiadiazol-2-yl)-N-[(3R)-3-piperidyl]benzamide (Example 710)

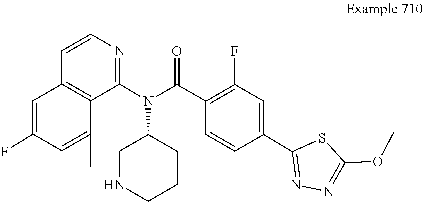 [SMILES: COc1nnc(-c2ccc(C(=O)N(c3nccc4cc(F)cc(C)c34)[C@@H]3CCCNC3)c(F)c2)s1]

Example 710 was prepared following the synthesis for example 833 by substituting 2-bromo-5-methyl-1,3,4-thiadiazole with 2-bromo-5-methoxy-1,3,4-thiadiazole, provided the title compound as a white solid LC-MS: [M+H⁺]=496.3. ¹H NMR (400 MHZ, CD₃OD): δ ppm 8.28-8.20 (m, 1H), 7.94 (s, 1H), 7.83-7.77 (m, 1H), 7.63 (s, 1H), 7.49-7.38 (m, 2H), 4.61 (br s, 1H), 3.81-3.70 (m, 1H), 3.62-3.57 (m, 2H), 3.50-3.45 (m, 1H), 3.38 (s, 3H), 3.20 (s, 1H), 3.17 (s, 1H), 3.23-3.12 (m, 1H), 3.07-2.92 (m, 1H), 2.76-2.68 (m, 2H), 2.49-2.46 (m, 3H), 2.06-1.96 (m, 3H), 1.73-1.63 (m, 2H), 1.34 (s, 6H), 1.29 (s, 1H), 0.96-0.79 (m, 1H).

### Synthesis of N-(6-chloro-8-methyl-1-isoquinolyl)-4-(1-ethyltriazol-4-yl)-2-fluoro-N-[(3R)-3-piperidyl] benzamide (Example 711)

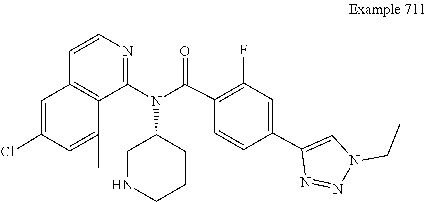 [SMILES: CCn1cc(-c2ccc(C(=O)N(c3nccc4cc(Cl)cc(C)c34)[C@@H]3CCCNC3)c(F)c2)nn1]

Example 711 was prepared following the procedure for example 444 by substituting 4-bromo-1-methyl-triazole with 4-bromo-1-ethyl-triazole to afford the title compound as an off-white solid. LC-MS: [M+H⁺]=493.3. ¹H NMR (400 MHZ, CD₃OD): δ ppm 8.49-8.41 (m, 1H), 8.26 (s, 1H), 7.74-7.62 (m, 2H), 7.49-7.43 (m, 1H), 7.31-7.25 (m, 1H), 7.19 (dd, J=1.3, 8.1 Hz, 1H), 6.93-6.81 (m, 1H), 5.11-5.03 (m, 1H), 4.41 (q, J=7.1 Hz, 2H), 3.99 (br d, J=9.8 Hz, 1H), 3.87-3.77 (m, 1H), 3.71-3.53 (m, 1H), 3.47-3.37 (m, 1H), 3.11-2.94 (m, 1H), 2.89-2.82 (m, 3H), 2.23-1.84 (m, 3H), 1.63-1.56 (m, 1H), 1.51 (t, J=7.3 Hz, 3H).

### Synthesis of 4-(1-ethyltriazol-4-yl)-2-fluoro-N-(6-fluoro-8-methyl-1-isoquinolyl)-N-[(3R)-3-piperidyl]benzamide (Example 712)

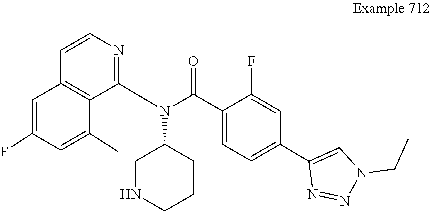 [SMILES: CCn1cc(-c2ccc(C(=O)N(c3nccc4cc(F)cc(C)c34)[C@@H]3CCCNC3)c(F)c2)nn1]

Example 712 was synthesized following the procedure for 833 by substituting 2-bromo-5-methyl-1,3,4-thiadiazole with 4-bromo-1-ethyl-triazole to afford the title product as a white solid. LC-MS: [M+H⁺]=477.3. ¹H NMR (400 MHZ, METHANOL-d₄): δ ppm 8.45-8.38 (m, 1H), 8.24 (s, 1H), 7.72-7.65 (m, 1H), 7.37-7.24 (m, 3H), 7.18 (dd, J=1.4, 8.1 Hz, 1H), 6.90-6.82 (m, 1H), 5.11-5.02 (m, 1H), 4.41 (q, J=7.4 Hz, 2H), 4.02-3.95 (m, 1H), 3.87-3.79 (m, 1H), 3.06-2.96 (m, 1H), 2.89-2.83 (m, 3H), 2.04-1.90 (m, 3H), 1.65-1.59 (m, 1H), 1.51 (t, J=7.4 Hz, 3H), 1.33-1.28 (m, 2H).

### Synthesis of (R)-4-((4-(4-ethylpiperazin-1-yl)pyrimidin-2-yl)amino)-2-fluoro-N-(8-methylisoquinolin-1-yl)-N-(piperidin-3-yl)benzamide (Example 713)

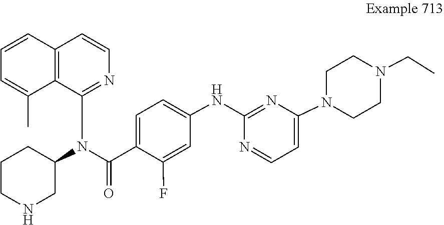 [SMILES: CCN1CCN(c2ccnc(Nc3ccc(C(=O)N(c4nccc5cccc(C)c45)[C@@H]4CCCNC4)c(F)c3)n2)CC1]

Example 713 was synthesized following the procedure for example 684 by substituting (R)-pyrrolidin-3-ol with 1-ethylpiperazine to afford the title product as a as pale-brown solid. LC-MS: [M+H⁺]=569.1. ¹H NMR (CD₃OD, 400 MHZ): δ ppm 8.45-8.41 (m, 1H), 7.90 (d, J=7.6 Hz, 1H), 7.78-7.76 (m, 1H), 7.70-7.66 (m, 1H), 7.55-7.46 (m, 2H), 7.09-7.02 (m, 1H), 6.90-6.80 (m, 2H), 6.71 (d, J=7.8 Hz, 1H), 5.09-5.02 (m, 1H), 4.80-4.71 (m, 1H), 4.46-4.44 (m, 1H), 3.99-3.96 (m, 1H), 3.80-3.71 (m, 4H), 3.52-3.38 (m, 3H), 3.23-3.28 (m, 3H), 3.02-2.98 (m, 1H), 2.82 (s, 3H), 2.69-2.68 (m, 1H), 1.96-1.88-1.89 (m, 2H), 1.56-1.51 (m, 1H). 1.45-1.41 (m, 3H).

### Synthesis of (R)-4-((4-(4-(dimethylamino) piperidin-1-yl)pyrimidin-2-yl)amino)-2-fluoro-N-(8-methylisoquinolin-1-yl)-N-(piperidin-3-yl)benzamide) (Example 714)

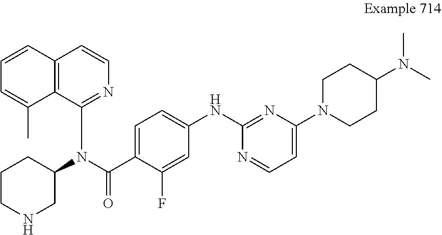 [SMILES: Cc1cccc2ccnc(N(C(=O)c3ccc(Nc4nccc(N5CCC(N(C)C)CC5)n4)cc3F)[C@@H]3CCCNC3)c12]

Example 714 was synthesized following the procedure for example 684 by substituting (R)-pyrrolidin-3-ol with N,N-dimethylpiperidin-4-amine to afford the title product as an off-white solid. LC-MS: [M+H⁺]=583.3. ¹H NMR (CD₃OD, 400 MHZ): δ ppm 8.45 (d, J=5.2 Hz, 1H), 7.81-7.73 (m, 2H), 7.70-7.66 (m, 1H), 7.54-7.47 (m, 2H), 71.3-7.05 (m, 1H), 6.86-6.77 (m, 2H), 6.67 (d, J=7.6 Hz, 1H), 5.08-5.02 (m, 1H), 4.83-4.79 (m, 1H), 4.38-4.33 (m, 2H), 3.98-3.96 (m, 1H), 3.83-3.80 (m, 1H), 344-3.38 (m, 2H), 3.06-2.98 (m, 2H), 2.85 (s, 6H), 2.82 (s, 3H), 2.69-2.68 (m, 1H), 2.28-2.25 (m, 2H), 1.94-1.88 (m, 2H), 1.77 (bs, 2H), 1.56 (bs, 1H).

### Synthesis of (R)-2-fluoro-N-(8-methylisoquinolin-1-yl)-N-(piperidin-3-yl)-4-((4-(4-(pyrrolidin-1-yl)piperidin-1-yl)pyrimidin-2-yl)amino)benzamide (Example 715)

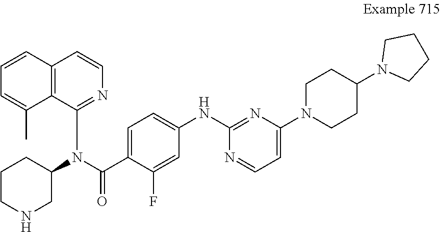 [SMILES: Cc1cccc2ccnc(N(C(=O)c3ccc(Nc4nccc(N5CCC(N6CCCC6)CC5)n4)cc3F)[C@@H]3CCCNC3)c12]

Example 715 was synthesized following the procedure for example 684 by substituting (R)-pyrrolidin-3-ol with 4-(pyrrolidin-1-yl)piperidine to afford the title compound as a pale-brown solid. LC-MS: [M+H⁺]=609.05. ¹H NMR (CD₃OD, 400 MHZ): δ ppm 8.44 (d, J=5.2 Hz, 1H), 7.81-7.73 (m, 2H), 7.70-7.65 (m, 1H), 7.54-7.47 (m, 2H), 7.13-7.05 (m, 1H), 6.86-6.77 (m, 2H), 6.66 (d, J=7.6 Hz, 1H), 5.08-5.02 (m, 1H), 4.83-4.73 (m, 1H), 4.35-4.32 (m, 1H), 3.99-3.86 (m, 1H), 3.83-3.80 (m, 1H), 3.62-3.52 (m, 4H), 3.41-3.38 (m, 1H), 3.24-3.19 (m, 2H), 3.06-3.01 (m, 2H), 2.82 (s, 3H), 2.70-2.68 (m, 1H), 2.34-2.31 (m, 2H), 2.18-2.16 (m, 2H), 2.07-2.06 (m, 2H), 1.94-1.88 (m, 2H), 1.81-1.78 (m, 2H), 1.28-1.27 9m, 1H).

### Synthesis of (R)-2-fluoro-4-((4-(4-methoxypiperidin-1-yl)pyrimidin-2-yl)amino)-N-(8-methylisoquinolin-1-yl)-N-(piperidin-3-yl)benzamide (Example 716)

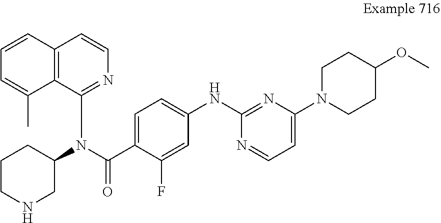 [SMILES: COC1CCN(c2ccnc(Nc3ccc(C(=O)N(c4nccc5cccc(C)c45)[C@@H]4CCCNC4)c(F)c3)n2)CC1]

Example 716 was synthesized following the procedure for example 684 by substituting (R)-pyrrolidin-3-ol with 4-methoxypiperidine hydrochloride to afford the title compound as a pale-brown solid. LC-MS: [M+H⁺]=570.1. ¹H NMR (CD₃OD, 400 MHZ): δ ppm 8.43-8.40 (m, 1H), 7.74-7.70 (m, 2H), 7.68-7.63 (m, 1H), 7.53-7.45 (m, 2H), 7.12-7.05 (m, 1H), 6.85-6.76 (m, 2H), 6.60 (d, J=7.6 Hz, 1H), 5.08-5.02 (m, 1H), 3.96-3.80 (m, 3H), 3.65-3.60 (m, 3H), 3.42-3.38 (m, 4H), 2.99-2.97 (m, 1H), 2.82 (s, 3H), 2.71-2.69 (m, 1H), 1.98-1.91 (m, 4H), 1.73-1.69 (m, 3H), 1.57-1.54 (m, 1H).

### Synthesis of (R)-2-fluoro-4-((4-(4-hydroxypiperidin-1-yl)pyrimidin-2-yl)amino)-N-(8-methylisoquinolin-1-yl)-N-(piperidin-3-yl)benzamide (Example 717)

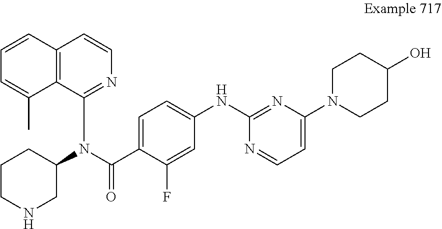 [SMILES: Cc1cccc2ccnc(N(C(=O)c3ccc(Nc4nccc(N5CCC(O)CC5)n4)cc3F)[C@@H]3CCCNC3)c12]

Example 717 was prepared according to the procedure described for Example 680 by substituting azetidin-3-ol with piperidin-4-ol, provided the title compound acetic acid salt as an off-white solid. LC-MS: [M+H⁺]=556.05. ¹H NMR (CD₃OD, 400 MHz): δ ppm 8.42-8.38 (m, 1H), 7.83 (d, J=6.4 Hz, 1H), 7.73-7.63 (m, 2H), 7.51-7.47 (m, 1H), 7.43-7.41 (m, 1H), 7.37-7.30 (m, 1H), 6.71 (d, J=8.8 Hz, 1H), 6.60-6.56 (m, 1H), 6.24 (d, J=6.0 Hz, 1H), 5.06-5.02 (m, 1H), 4.06-4.03 (m, 2H), 3.89-3.87 (m, 2H), 3.78-3.76 (m, 1H), 3.24-3.18 (m, 2H), 2.93-2.89 (m, 2H), 2.79 (s, 3H), 1.94-1.87 (m, 5H), 1.47-1.42 (m, 3H).

### Synthesis of (R)-2-fluoro-N-(8-methylisoquinolin-1-yl)-4-((4-(piperazin-1-yl)pyrimidin-2-yl)amino)-N-(piperidin-3-yl)benzamide (Example 718)

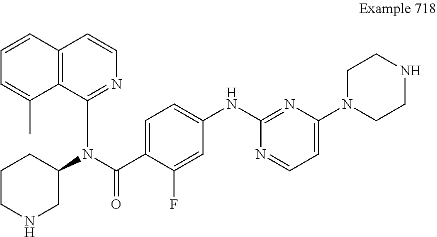 [SMILES: Cc1cccc2ccnc(N(C(=O)c3ccc(Nc4nccc(N5CCNCC5)n4)cc3F)[C@@H]3CCCNC3)c12]

Example 718 was synthesized following the procedure for example 684 by substituting (R)-pyrrolidin-3-ol with tert-butyl piperazine-1-carboxylate to afford the title compound as an off-white solid. LC-MS: [M+H⁺]=541.3. ¹H NMR (CD₃OD, 400 MHZ): δ ppm 8.44-8.41 (m, 1H), 7.88 (d, J=7.6 Hz, 1H), 7.76-7.67 (m, 2H), 7.54-7.46 (m, 1H), 7.10-7.7.03 (m, 1H), 6.87-6.79 (m, 2H), 6.69 (d, J=7.2 Hz, 1H), 5.08-5.04 (m, 1H), 4.02-3.96 (m, 5H), 3.82-3.76 (m, 1H), 3.59-3.38 (m, 5H), 3.01-2.92 (m, 1H), 2.82 (s, 3H), 2.69-2.68 (m, 1H), 1.98-1.94 (m, 2H), 1.51-1.50 (m, 1H).

### Synthesis of (R)-2-fluoro-N-(8-methylisoquinolin-1-yl)-4-((4-(4-oxopiperidin-1-yl)pyrimidin-2-yl)amino)-N-(piperidin-3-yl)benzamide (Example 719)

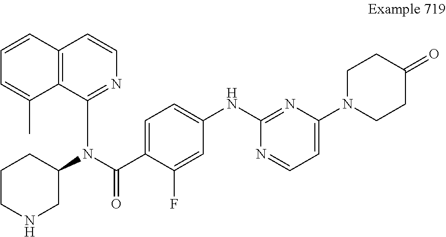 [SMILES: Cc1cccc2ccnc(N(C(=O)c3ccc(Nc4nccc(N5CCC(=O)CC5)n4)cc3F)[C@@H]3CCCNC3)c12]

Example 719 was synthesized following the procedure for example 684 by substituting (R)-pyrrolidin-3-ol with piperidin-4-one hydrochloride to afford the title compound as an off-white solid. LC-MS: [M+H⁺]=554.3. ¹H NMR (CD₃OD, 400 MHZ): δ ppm 8.43 (d, J=5.6 Hz, 1H), 7.74-7.63 (m, 3H), 7.53-7.45 (m, 2H), 7.15-7.01 (m, 1H), 6.86-6.76 (m, 2H), 6.62 (d, J=6.8 Hz, 1H), 5.08-5.03 (m, 1H), 4.01-3.96 (m, 2H), 3.83-3.77 (m, 4H), 3.47-3.38 (m, 1H), 3.01-2.96 (m, 1H), 2.82 (s, 3H), 2.70-2.69 (m, 1H), 1.94-1.79 (m, 6H), 1.57-1.53 9m, 1H).

### Synthesis of (R)-4-((4-(6-aminopyridin-3-yl)pyrimidin-2-yl)amino)-2-fluoro-N-(8-methylisoquinolin-1-yl)-N-(piperidin-3-yl)benzamide (Example 720)

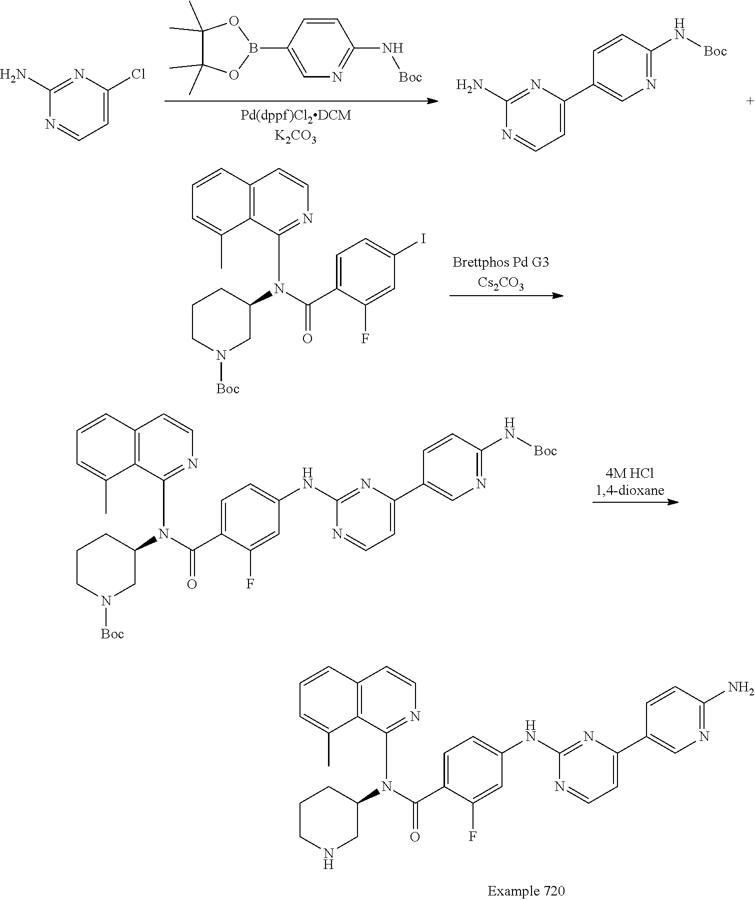

Example 720 was prepared according to the procedure for Example 679 by substituting 4,4,5,5-tetramethyl-2-(1,4-dioxaspiro[4.5]dec-7-en-8-yl)-1,3,2-dioxaborolane with tert-butyl(5-(4,4,5,5-tetramethyl-1,3,2-dioxaborolan-2-yl)pyridin-2-yl)carbamate, provided the title compound as an off-white solid. LC-MS: [M+H⁺]=549.3. ¹H NMR (CD₃OD, 400 MHZ): δ ppm 8.60 (bs, 1H), 8.55 (d, J=9.2 Hz, 1H), 8.50 (d, J=5.2 Hz, 1H), 8.45-8.41 (m, 1H), 7.77-7.72 (m, 1H), 7.68-7.64 (m, 1H), 7.52-7.43 (m, 3H), 7.36 (d, J=5.2 Hz, 1H), 7.15 (d, J=9.6 Hz, 1H), 6.87 (d, J=8.4 Hz, 1H), 6.73-6.69 (m, 1H), 5.09-5.03 (m, 1H), 3.98-3.95 (m, 1H), 3.85-3.79 (m, 1H), 3.47-3.38 (m, 1H), 3.02-2.99 (m, 1H), 2.81 (s, 3H), 2.70-2.69 (m, 1H), 1.94-1.87 (m, 2H), 1.63-1.54 (m, 1H).

### Synthesis of (R)-2-fluoro-4-((4-(4-hydroxyphenyl)pyrimidin-2-yl)amino)-N-(8-methylisoquinolin-1-yl)-N-(piperidin-3-yl)benzamide (Example 721)

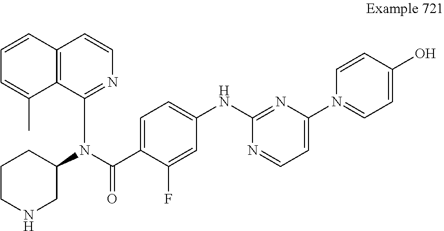

Example 721 was synthesized following the procedure for example 684 by substituting (R)-pyrrolidin-3-ol with 4-(2-aminopyrimidin-4-yl)phenol to afford the title compound as an off-white solid. LC-MS: [M+H⁺]=549.3. ¹H NMR (CD₃OD, 400 MHZ): δ ppm 8.47-8.43 (m, 1H), 8.29 (d, J=6.4 Hz, 1H), 8.03 (d, J=8.8 Hz, 1H), 7.75-7.71 (m, 1H), 7.65-7.63 (m, 1H), 7.56 (d, J=6.4 Hz, 1H), 7.49-7.47 (m, 2H), 7.32-7.24 (m, 1H), 6.98-6.93 (m, 2H), 6.91-6.84 (m, 2H), 5.10-5.02 (m, 1H), 4.01-3.98 (m, 1H), 3.85-3.80 (m, 1H), 3.45-3.39 (m, 1H), 3.03-2.99 (m, 1H), 2.84 (s, 3H), 2.74-2.69 (m, 1H), 1.96-1.90 (m, 2H), 1.32-1.28 (m, 1H).

### Synthesis of (R)-4-((4-(1H-1,2,4-triazol-1-yl)pyrimidin-2-yl)amino)-2-fluoro-N-(8-methylisoquinolin-1-yl)-N-(piperidin-3-yl)benzamide (Example 722)

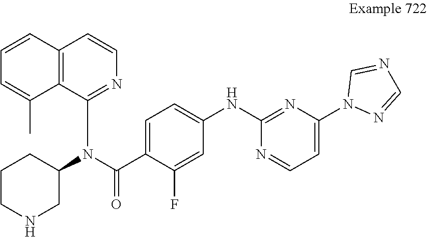 [SMILES: Cc1cccc2ccnc(N(C(=O)c3ccc(Nc4nccc(-n5cncn5)n4)cc3F)[C@@H]3CCCNC3)c12]

Example 722 was synthesized following the procedure for example 680 by substituting azetidin-3-ol hydrochloride with (1H)-1,2,4-triazole to afford the title compound as an off-white solid. LC-MS: [M+H⁺]=524.3. ¹H NMR (CD₃OD, 400 MHZ): δ ppm 9.20 (bs, 1H), 8.58 (d, J=5.2 Hz, 1H), 8.45-8.41 (m, 1H), 8.24 (s, 1H), 7.76-7.71 (m, 1H), 7.66-7.62 (m, 1H), 7.51-7.39 (m, 3H), 7.32 (d, J=5.6 Hz, 1H), 6.92-6.89 (m, 1H), 6.72-6.68 (m, 1H), 5.10-5.02 (m, 1H), 3.98-3.95 (m, 1H), 3.85-3.80 (m, 1H), 3.44-3.38 (m, 1H), 3.03-2.99 (m, 1H), 2.81 (s, 3H), 2.70-2.69 (m, 1H), 1.98-1.88 (m, 2H), 1.61-1.57 (m, 1H).

### Synthesis of (R)-4-((4-(1H-pyrazol-1-yl)pyrimidin-2-yl)amino)-2-fluoro-N-(8-methylisoquinolin-1-yl)-N-(piperidin-3-yl)benzamide (Example 723)

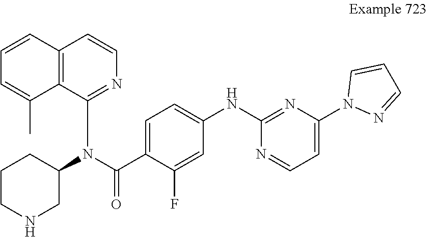 [SMILES: Cc1cccc2ccnc(N(C(=O)c3ccc(Nc4nccc(-n5cccn5)n4)cc3F)[C@@H]3CCCNC3)c12]

Example 723 was synthesized following the procedure for example 680 by substituting azetidin-3-ol hydrochloride with 1H-pyrazole to afford the title compound as an off-white solid. LC-MS: [M−HCl+H⁺]=523.3. ¹H NMR (CD₃OD, 400 MHZ): δ ppm 8.45-8.41 (m, 3H), 7.84 (s, 1H), 7.75-7.71 (m, 1H), 7.67-7.65 (m, 1H), 7.51-7.43 (m, 2H), 7.39-7.35 (m, 2H), 6.89-6.87 (m, 1H), 6.73-6.69 (m, 1H), 6.63-6.62 (m, 1H), 5.11-5.03 (m, 1H), 3.98-3.95 (m, 1H), 3.85-3.79 (m, 1H), 3.48-3.38 (m, 1H), 3.02-2.99 (m, 1H), 2.81 (s, 3H), 2.70-2.67 (m, 1H), 1.98-1.91 (m, 2H), 1.60-1.56 (m, 1H).

### Synthesis of (R)-4-((4-(1H-imidazol-1-yl)pyrimidin-2-yl)amino)-2-fluoro-N-(8-methylisoquinolin-1-yl)-N-(piperidin-3-yl)benzamide (Example 724)

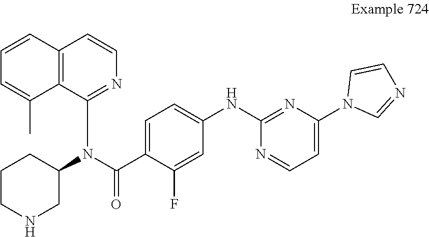 [SMILES: Cc1cccc2ccnc(N(C(=O)c3ccc(Nc4nccc(-n5ccnc5)n4)cc3F)[C@@H]3CCCNC3)c12]

Example 724 was synthesized following the procedure for 680 by substituting azetidin-3-ol hydrochloride with 1H-imidazole to afford the title compound as an off-white solid. LC-MS: [M+H⁺]=523.3. ¹H NMR (CD₃OD, 400 MHZ): δ ppm 8.64 (d, J=5.6 Hz, 1H), 8.45-8.41 (m, 1H), 8.19-8.17 (m, 1H), 7.75-7.71 (m, 2H), 7.67-7.62 (m, 2H), 7.49-7.44 (m, 3H), 7.26 (s, J=5.2 Hz, 1H), 6.90-6.88 (m, 1H), 6.73-6.69 (m, 1H), 5.09-5.03 (m, 1H), 3.98-3.95 (m, 1H), 3.84-3.79 (m, 1H), 3.48-3.47 (m, 1H), 3.13-3.12 (m, 1H), 2.81 (s, 3H), 2.70-2.66 (m, 1H), 1.98-1.91 (m, 2H), 1.59-1.55 (m, 1H).

### Synthesis of N-(6-fluoro-8-methyl-1-isoquinolyl)-4-(5-methyl-1,3,4-thiadiazol-2-yl)-N-(3-piperidyl)benzamide (Example 725)

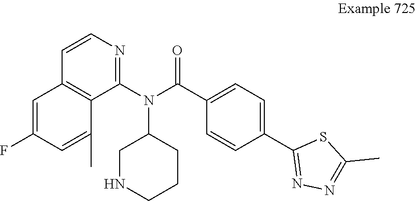 [SMILES: Cc1nnc(-c2ccc(C(=O)N(c3nccc4cc(F)cc(C)c34)C3CCCNC3)cc2)s1]

Example 725 was synthesized following the synthesis for example 833 by substituting 2-Fluoro-4-iodobenzoyl chloride with 4-iodobenzoyl chloride to afford the title compound as a brown solid. LC-MS: [M+H]=462.3. ¹H NMR (400 MHZ, CD₃OD): δ ppm 1.82-1.90 (m, 1H) 1.91-2.01 (m, 2H) 2.76 (d, J=7.00 Hz, 6H) 2.83 (s, 1H) 2.94-3.06 (m, 1H) 3.40 (br d, J=13.13 Hz, 2H) 3.75-3.88 (m, 1H) 3.96 (br d, J=10.63 Hz, 1H) 5.04-5.16 (m, 1H) 7.13-7.20 (m, 2H) 7.21-7.27 (m, 1H) 7.34-7.40 (m, 1H) 7.54-7.61 (m, 2H) 7.74-7.82 (m, 1H) 8.45-8.54 (m, 1H).

### Synthesis of (R)-2-fluoro-4-((3-(methylamino)pyridin-2-yl)amino)-N-(8-methylisoquinolin-1-yl)-N-(piperidin-3-yl)benzamide (Example 726)

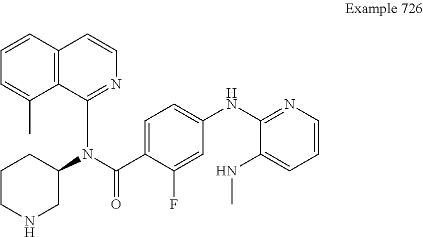 [SMILES: CNc1cccnc1Nc1ccc(C(=O)N(c2nccc3cccc(C)c23)[C@@H]2CCCNC2)c(F)c1]

Example 726 was synthesized following the procedure for example 210 by substituting 2-chloropyrimidine with 2-chloro-N-methylpyridin-3-amine to afford the title compound as a light brown solid. LC-MS: [M+H⁺]=485.2. ¹H NMR (CD₃OD, 400 MHZ): δ ppm 8.44-8.40 (m, 1H), 7.77-7.67 (m, 2H), 7.56-7.51 (m, 1H), 7.48 (d, J=7.2 Hz, 1H), 7.32-7.29 (m, 2H), 7.25-7.21 (m, 1H), 6.94-6.90 (m, 1H), 6.78-6.72 (m, 1H), 6.57-6.55 (m, 1H), 5.08-5.01 (m, 1H), 3.98-3.95 (m, 1H), 3.79-3.73 (m, 1H), 3.39-3.36 (m, 1H), 3.02-2.95 (m, 1H), 2.95 (s, 3H), 2.82 (s, 3H), 2.68-2.62 (m, 1H), 1.94-1.84 (m, 2H), 1.48-1.37 (m, 1H).

### Synthesis of (R)-2-fluoro-N-(8-methylisoquinolin-1-yl)-N-(piperidin-3-yl)-4-((4-(pyridin-3-yl)pyrimidin-2-yl)amino)benzamide (Example 727)

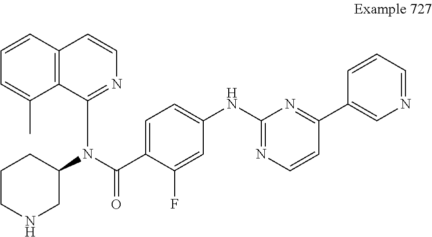 [SMILES: Cc1cccc2ccnc(N(C(=O)c3ccc(Nc4nccc(-c5cccnc5)n4)cc3F)[C@@H]3CCCNC3)c12]

Example 727 was synthesized following the procedure for example 131 by substituting 3-amino-1-methylpyridin-2 (1H)-one with 4-(pyridin-3-yl)pyrimidin-2-amine to afford the title compound as a yellow solid. LC-MS: [M+H⁺]=534.3. ¹H NMR (CD₃OD, 400 MHz): δ ppm 9.50 (bs, 1H), 9.27 (d, J=8.0 Hz, 1H), 8.99 (d, J=6.0 Hz, 1H), 8.66 (d, J=5.2 Hz, 1H), 8.45-8.41 (m, 1H), 8.28-8.24 (m, 1H), 7.76-7.72 (m, 1H), 7.68-7.63 (m, 1H), 7.57-7.54 (m, 1H), 7.52-7.44 (m, 3H), 6.91 (d, J=8.4 Hz, 1H), 6.73-6.68 (m, 1H), 5.10-5.04 (m, 1H), 3.98-3.95 (m, 1H), 3.85-3.79 (m, 1H), 3.47-3.38 (m, 1H), 3.02-2.99 (m, 1H), 2.81 (s, 3H), 2.69-2.68 (m, 1H), 1.95-1.88 (m, 2H), 1.59-1.56 (m, 1H).

### Synthesis of (R)-2-fluoro-N-(8-methylisoquinolin-1-yl)-N-(piperidin-3-yl)-4-((4-(pyridin-2-yl)pyrimidin-2-yl)amino)benzamide (Example 728)

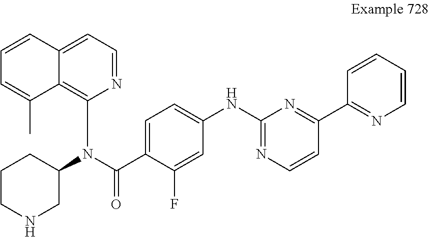 [SMILES: Cc1cccc2ccnc(N(C(=O)c3ccc(Nc4nccc(-c5ccccn5)n4)cc3F)[C@@H]3CCCNC3)c12]

Example 728 was synthesized following the procedure for example 131 by substituting 3-amino-1-methylpyridin-2 (1H)-one with 4-(pyridin-2-yl)pyrimidin-2-amine to afford the title compound as a yellow solid. LC-MS: [M+H⁺]=534.5. ¹H NMR (CD₃OD, 400 MHz): δ ppm 8.96 (d, J=5.6 Hz, 1H), 8.78-8.71 (m, 3H), 8.45-8.41 (m, 1H), 8.20-8.17 (m, 1H), 7.77-7.72 (m, 2H), 7.68-7.64 (m, 1H), 7.56-7.45 (m, 3H), 6.95 (d, J=8.8 Hz, 1H), 6.77-6.72 (m, 1H), 5.11-5.03 (m, 1H), 3.99-3.98 (m, 1H), 3.85-3.79 (m, 1H), 3.45-3.38 (m, 1H), 3.02-2.97 (m, 1H), 2.82 (s, 3H), 2.70-2.69 (m, 1H), 1.95-1.87 (m, 2H), 1.58-1.53 (m, 1H).

### Synthesis of (R)—N-(6-fluoro-8-methylisoquinolin-1-yl)-5-(1-(2-methoxyethyl)-3-methyl-1H-pyrazol-5-yl)-N-(piperidin-3-yl)picolinamide (Example 729)

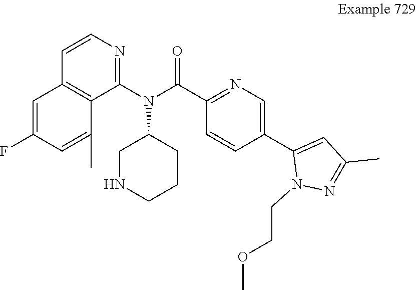 [SMILES: COCCn1nc(C)cc1-c1ccc(C(=O)N(c2nccc3cc(F)cc(C)c23)[C@@H]2CCCNC2)nc1]

Example 729 was prepared according to the procedure described for Example 672 by substituting 2-bromo-5-ethyl-1,3,4-thiadiazole with 5-bromo-1-(2-methoxyethyl)-3-methyl-pyrazole to afford the title compound as a yellow solid. LC-MS: [M+H⁺]=503.4. ¹H NMR (400 MHZ, DMSO-d₆): δ 9.25-8.83 (m, 2H), 8.36 (s, 1H), 7.95-7.91 (m, 1H), 7.87-7.76 (m, 2H), 7.73 (d, J=8.3 Hz, 1H), 7.63 (br d, J=9.0 Hz, 1H), 7.47-7.35 (m, 1H), 7.32-7.02 (m, 1H), 6.09 (s, 1H), 3.90-3.77 (m, 3H), 3.54-3.38 (m, 3H), 3.26-3.17 (m, 1H), 3.00-2.92 (m, 3H), 2.88-2.66 (m, 4H), 2.11 (s, 3H), 1.86-1.65 (m, 3H), 1.16-1.03 (m, 1H).

### Synthesis of (R)—N-(6-chloro-8-methylisoquinolin-1-yl)-5-(5-ethyl-1,3,4-thiadiazol-2-yl)-N-(piperidin-3-yl)picolinamide (Example 730)

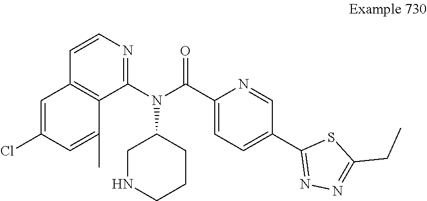 [SMILES: CCc1nnc(-c2ccc(C(=O)N(c3nccc4cc(Cl)cc(C)c34)[C@@H]3CCCNC3)nc2)s1]

Example 730 was prepared according to the procedure described for Example 816 by substituting 4-bromo-2-chloro-benzoyl chloride and 2-bromo-5-methyl-1, 3, 4-thiadiazole with 5-bromopyridine-2-carbonyl chloride and 2-bromo-5-ethyl-1, 3, 4-thiadiazole, provided the title compound hydrochloride salt as a white solid. LC-MS: [M+H⁺]=493.3. ¹H NMR (400 MHZ, CD₃OD): δ ppm 8.43-8.32 (m, 3H), 8.09 (br d, J=8.1 Hz, 1H), 8.03-7.94 (m, 2H), 7.65-7.55 (m, 1H), 5.04-4.95 (m, 1H), 4.02 (br d, J=11.8 Hz, 1H), 3.51 (t, J=11.5 Hz, 1H), 3.35 (br s, 1H), 3.15 (q, J=7.5 Hz, 2H), 2.91 (s, 1H), 2.79 (s, 3H), 1.95-1.74 (m, 3H), 1.40 (t, J=7.6 Hz, 3H), 1.08 (tt, J=8.3, 12.3 Hz, 1H).

### Synthesis of (R)-4-((4-(1-chloro-3-methoxyprop-1-en-1-yl)pyrimidin-2-yl)amino)-2-fluoro-N-(8-methylisoquinolin-1-yl)-N-(piperidin-3-yl)benzamide (Example 731)

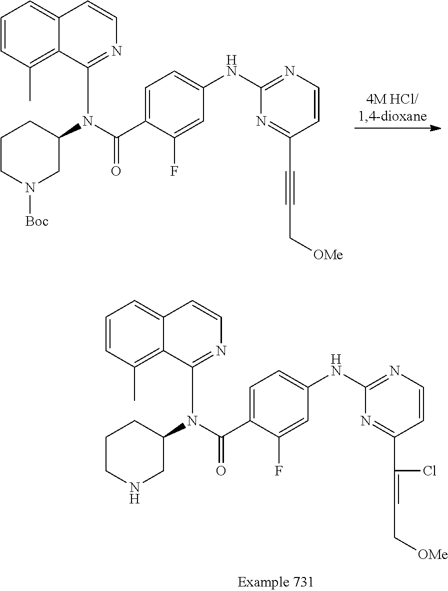 [SMILES: COCC#Cc1ccnc(Nc2ccc(C(=O)N(c3nccc4cccc(C)c34)[C@@H]3CCCN(C(=O)OC(C)(C)C)C3)c(F)c2)n1 ; COCC=C(Cl)c1ccnc(Nc2ccc(C(=O)N(c3nccc4cccc(C)c34)[C@@H]3CCCNC3)c(F)c2)n1]

To a stirred solution of tert-butyl (R)-3-(2-fluoro-4-((4-(3-methoxyprop-1-yn-1-yl) pyrimidin-2-yl) amino)-N-(8-methylisoquinolin-1-yl)benzamido)piperidine-1-carboxylate (60 mg, 0.096 mmol) in CH₂Cl₂ (1 mL) was added 4M HCl in 1,4-dioxane (2 mL) at 0° C. and stirred at room temperature for 2 h. The reaction mixture was concentrated under reduced pressure. The obtained compound was purified by prep HPLC to afford the title compound formic acid salt as an off-white solid. LC-MS: [M+H⁺]=562.2. ¹H NMR (CD₃OD, 400 MHz): δ ppm 8.42-8.38 (m, 2H), 7.73-7.64 (m, 1H), 7.60-7.56 (m, 2H), 7.50-7.42 (m, 2H), 7.24 (d, J=5.2 Hz, 1H), 6.85-6.83 (m, 1H), 6.80-6.78 (m, 1H), 6.65-6.59 (m, 1H), 5.02-5.01 (m, 1H), 4.18 (s, 2H), 3.90-3.87 (m, 1H), 3.74-3.68 (m, 1H), 3.46 (s, 3H), 3.34-3.33 (m, 1H), 3.28-3.21 (m, 1H), 2.92-2.89 (m, 1H), 2.80 (s, 3H), 1.90-1.86 (m, 2H), 1.60-1.59 (m, 1H).

### Synthesis of 2-fluoro-N-[8-methyl-6-(2-methylthiazol-5-yl)-1-isoquinolyl]-4-(5-methyl-1,3,4-thiadiazol-2-yl)-N-[(3R)-3-piperidyl]benzamide (Example 732)

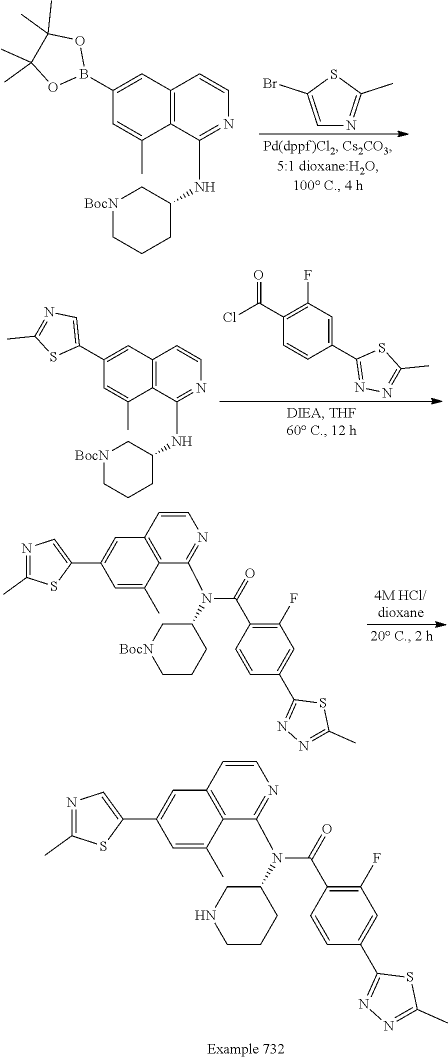

### Step 1:

To a mixture of tert-butyl (3R)-3-[[8-methyl-6-(4,4,5,5-tetramethyl-1,3,2-dioxaborolan-2-yl)-1-isoquinolyl] amino] piperidine-1-carboxylate (500 mg, 1.07 mmol), 5-bromo-2-methyl-thiazole (285.70 mg, 1.60 mmol), and Cs₂CO₃ (1.05 g, 3.21 mmol) in dioxane (2 mL) and H₂O (0.4 mL) at 20° C. was added Pd(dppf)Cl₂ (78.27 mg, 106.97 μmol) under N₂ atmosphere, and then the mixture was heated to 100° C. and stirred for 4 h under N₂ atmosphere. The reaction mixture was filtered and concentrated under reduced pressure to give a residue. The residue was purified by column chromatography (SiO₂, Petroleum ether/EtOAc=3/1 to 2/1) to give tert-butyl (3R)-3-[[8-methyl-6-(2-methylthiazol-5-yl)-1-isoquinolyl]amino]piperidine-1-carboxylate (230 mg, 47%) as a yellow oil. LC-MS: [M+H⁺]=439.3.

A mixture of tert-butyl (3R)-3-[8-methyl-6-(2-methylthiazol-5-yl)-1-isoquinolyl]-amino]piperidine-1-carboxylate (200 mg, 456.01 μmol), 2-fluoro-4-(5-methyl-1,3,4-thiadiazol-2-yl)benzoylchloride (351.15 mg, 1.37 mmol), and DIEA (294.68 mg, 2.28 mmol, 397.15 μL) in THF (2 mL) was degassed and purged with N₂ 3 times at 20° C., and then the mixture was heated to 60° C. and stirred for 12 h under N₂ atmosphere. The reaction mixture was filtered and concentrated under reduced pressure to give a residue. The residue was purified by column chromatography to give tert-butyl (3R)-3-[[2-fluoro-4-(5-methyl-1,3,4-thiadiazol-2-yl)benzoyl]-[8-methyl-6-(2-methylthiazol-5-yl)-1-isoquinolyl]amino]piperidine-1-carboxylate (111 mg, 24%) as a yellow oil. LC-MS: [M+H⁺]=659.3.

### Step 3:

To a solution of tert-butyl (3R)-3-[[2-fluoro-4-(5-methyl-1,3,4-thiadiazol-2-yl) benzoyl]-[8-methyl-6-(2-methylthiazol-5-yl)-1-isoquinolyl]amino]piperidine-1-carboxylate (100 mg, 151.79 μmol) in dioxane (2 mL) was added 4M HCl in dioxane (3.00 mL). The mixture was stirred at 20° C. for 2 h. The reaction mixture was filtered and concentrated under reduced pressure to give a residue, which was purified by prep-HPLC to give the title compound hydrochloride salt (34 mg, 38%) as a yellow solid. LC-MS: [M+H⁺]=559.3. ¹H NMR (400 MHZ, CD₃OD): δ=8.52-8.44 (m, 1H), 8.42-8.37 (m, 1H), 7.99-7.92 (m, 1H), 7.84-7.75 (m, 2H), 7.45-7.34 (m, 1H), 7.29 (dd, J=1.6, 8.1 Hz, 1H), 7.06-6.97 (m, 1H), 5.17-5.01 (m, 1H), 4.09-3.94 (m, 1H), 3.89-3.77 (m, 1H), 3.49-3.39 (m, 1H), 3.11-2.93 (m, 2H), 2.90 (d, J=4.6 Hz, 5H), 2.73 (s, 3H), 2.14-1.81 (m, 3H), 1.71-1.51 (m, 1H).

### Synthesis of 2-fluoro-4-(5-methyl-1,3,4-thiadiazol-2-yl)-N-(8-methyl-6-thiazol-2-yl-1-isoquinolyl)-N-[rac-(3R)-3-piperidyl] benzamide (Example 733)

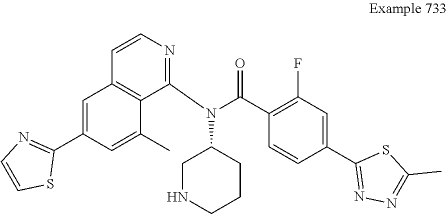 [SMILES: Cc1nnc(-c2ccc(C(=O)N(c3nccc4cc(-c5nccs5)cc(C)c34)[C@@H]3CCCNC3)c(F)c2)s1]

Example 733 was prepared according to the procedure described for Example 732 by substituting 5-bromo-2-methyl-thiazole with 2-bromothiazole to afford the title compound as an off-white solid. LC-MS: [M+H⁺]=545.3. ¹H NMR (CD₃OD, 400 MHZ): δ ppm 8.58-8.40 (m, 1H), 8.33-8.18 (m, 1H), 8.11-7.99 (m, 2H), 7.93-7.77 (m, 2H), 7.45-7.34 (m, 1H), 7.29 (d, J=8.1 Hz, 1H), 7.10-6.96 (m, 1H), 5.18-5.02 (m, 1H), 4.09-3.95 (m, 1H), 3.91-3.76 (m, 1H), 3.49-3.38 (m, 1H), 3.14-2.98 (m, 1H), 2.98-2.90 (m, 3H), 2.72 (s, 3H), 2.17-1.80 (m, 3H), 1.75-1.52 (m, 1H).

### Synthesis of (R)-2-fluoro-4-((4-((2-hydroxyethyl)amino)pyrimidin-2-yl)amino)-N-(8-methylisoquinolin-1-yl)-N-(piperidin-3-yl)benzamide (Example 734)

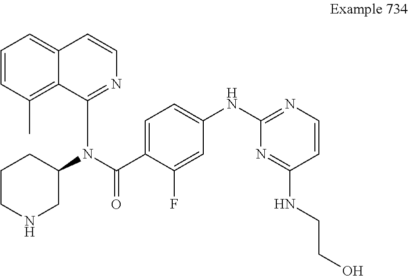 [SMILES: Cc1cccc2ccnc(N(C(=O)c3ccc(Nc4nccc(NCCO)n4)cc3F)[C@@H]3CCCNC3)c12]

Example 734 was synthesized following the procedure for example 131 by substituting 3-amino-1-methylpyridin-2 (1H)-one with 2-((2-aminopyrimidin-4-yl) amino) ethan-1-ol to afford the title compound as an off-white solid. LC-MS: [M+H⁺]=516.3. ¹H NMR (CD₃OD, 400 MHz): δ ppm 8.43-8.40 (m, 1H), 7.74-7.60 (m, 3H), 7.51-7.45 (m, 2H), 7.22-7.15 (m, 1H), 6.82-6.81 (m, 2H), 6.24 (d, J=7.2 Hz, 1H), 5.08-5.02 (m, 1H), 3.96-3.95 (m, 1H), 3.83-3.80 (m, 1H), 3.70-3.67 (m, 2H), 3.49-3.38 (m, 3H), 2.99-2.96 (m, 1H), 2.81 (s, 3H), 1.96-1.91 (m, 3H), 1.57-1.53 (m, 1H).

### Synthesis of (R)-2-fluoro-4-((4-hydroxypyrimidin-2-yl)amino)-N-(8-methylisoquinolin-1-yl)-N-(piperidin-3-yl)benzamide (Example 735)

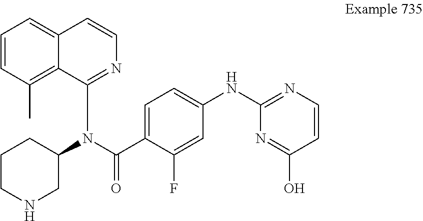 [SMILES: Cc1cccc2ccnc(N(C(=O)c3ccc(Nc4nccc(O)n4)cc3F)[C@@H]3CCCNC3)c12]

Example 735 was synthesized following the procedure for example 131 by substituting 3-amino-1-methylpyridin-2 (1H)-one with 2-aminopyrimidin-4-ol to afford the title compound as a pale brown solid. LC-MS: [M+1⁺]=473.1. ¹H NMR (CD₃OD, 400 MHz): δ ppm 8.43-8.4 (m, 1H), 7.95 (d, J=7.2 Hz, 1H), 7.75-7.66 (m, 3H), 7.53-7.34 (m, 3H), 6.87-6.80 (m, 2H), 6.31 (d, J=7.2 Hz, 1H), 5.08-5.02 (m, 1H), 3.99-3.96 (m, 1H), 3.80-3.76 (m, 1H), 3.48-3.45 (m, 1H), 3.13-3.12 (m, 1H), 3.01-2.98 (m, 1H), 2.81 (s, 3H), 2.79-2.78 (m, 1H), 1.95-1-90 (m, 1H).

### Synthesis of (R)-2-fluoro-4-((4-(2-hydroxyethyl)pyrimidin-2-yl)amino)-N-(8-methylisoquinolin-1-yl)-N-(piperidin-3-yl)benzamide (Example 736)

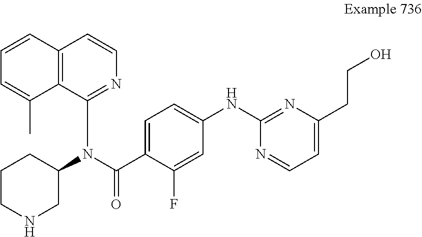 [SMILES: Cc1cccc2ccnc(N(C(=O)c3ccc(Nc4nccc(CCO)n4)cc3F)[C@@H]3CCCNC3)c12]

Example 736 was synthesized following the procedure for example 131 by substituting 3-amino-1-methylpyridin-2 (1H)-one with 2-amino-4-pyrimidineethanol to afford the title compound as a pale-yellow solid. LC-MS: [M+H⁺]=501.2. ¹H NMR (CD₃OD, 400 MHz): δ ppm 8.43-8.40 (m, 1H), 8.31-8.26 (m, 1H), 7.74-7.72 (m, 1H), 7.67-7.63 (m, 1H), 7.57-7.42 (m, 3H), 6.85-6.75 (m, 2H), 6.63-6.58 (m, 2H), 5.09-5.02 (m, 1H), 4.85-4.73 (m, 1H), 3.96-3.88 (m, 2H), 3.84-3.78 (m, 1H), 3.40-3.37 (m, 1H), 3.25-3.09 (m, 1H), 3.08-2.98 (m, 1H), 2.82-2.79 (m, 4H), 1.94-1.90 (m, 3H), 1.62-1.55 (m, 1H).

### Synthesis of (R)-4-(5,5-dimethyl-4-oxo-4,5-dihydroisoxazol-3-yl)-N-(2-(3-hydroxyprop-1-yn-1-yl)thieno[3,2-c]pyridin-4-yl)-2-methyl-N-(piperidin-3-yl)benzamide (Example 737)

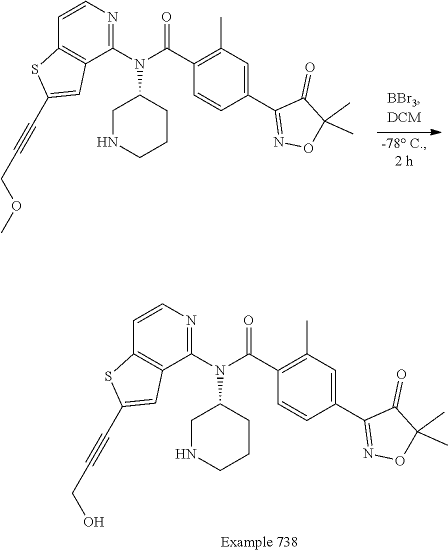

To a solution of (R)-4-(5,5-dimethyl-4-oxo-4,5-dihydroisoxazol-3-yl)-N-(2-(3-methoxyprop-1-yn-1-yl)thieno[3,2-c]pyridin-4-yl)-2-methyl-N-(piperidin-3-yl)benzamide (50 mg, 94.23 μmol) in DCM (0.5 mL) at −78° C. under N₂ atmosphere was added BBr₃ (90.79 μL, 942.26 μmol). The mixture was stirred at −78° C. for 2 h and then was quenched by addition 1M aq. HCl solution (1 mL) at 0° C. The reaction mixture was extracted with EtOAc 30 mL (2×10 mL). The combined organic layer was washed with brine (5 mL), dried over Na₂SO₄, filtered and concentrated. The residue was purified by prep-HPLC (column: 3_Phenomenex Luna C18 75*30 mm*3 μm; mobile phase: [H₂O (0.04% HCl)-MeCN]; gradient: 20%-65% B over 8.0 min) and the pure fraction was lyophilized to give the title compound hydrochloride salt (5 mg, 10%) as a white solid. LC-MS: [M+H⁺]=517.3. ¹H NMR (400 MHZ, CD₃OD): δ ppm 8.33 (br d, J=5.4 Hz, 1H), 7.78 (br d, J=5.3 Hz, 1H), 7.71 (br s, 2H), 7.50 (br d, J=8.1 Hz, 1H), 7.12 (br d, J=7.8 Hz, 1H), 5.28-4.98 (m, 1H), 4.47 (s, 2H), 4.24 (s, 1H), 3.89 (br d, J=10.8 Hz, 1H), 3.61-3.49 (m, 1H), 3.34 (br d, J=2.6 Hz, 1H), 2.93-2.74 (m, 1H), 2.47 (s, 3H), 2.22-1.76 (m, 3H), 1.35 (s, 6H), 1.32-1.14 (m, 1H).

### Synthesis of (R)—N-(6-chloroisoquinolin-1-yl)-2-fluoro-N-(piperidin-3-yl)-4-(pyrimidin-2-ylamino)benzamide (Example 794) & (R)-2-fluoro-N-(piperidin-3-yl)-4-(pyrimidin-2-ylamino)-N-(6-(pyrimidin-2-ylamino) isoquinolin-1-yl)benzamide (Example 738)

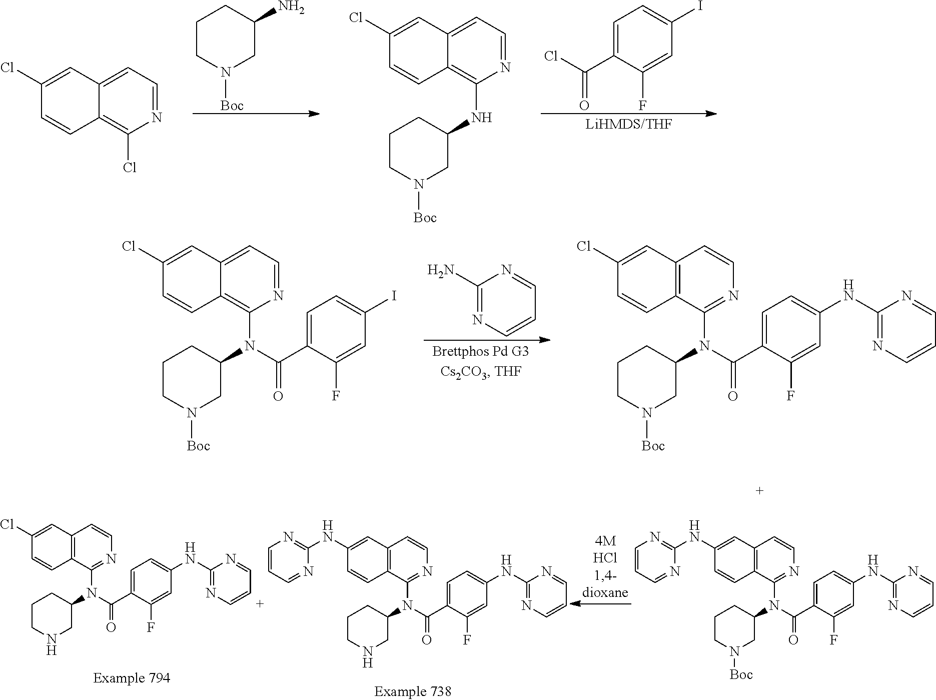

### Step 1:

A mixture of 1,6-dichloroisoquinoline (3 g, 15.15 mmol) and tert-butyl (R)-3-aminopiperidine-1-carboxylate (6.07 g, 30.3 mmol) were stirred at 140° C. for 16 h. The reaction was concentrated. The residue was purified by Combi-flash to afford tert-butyl (R)-3-((6-chloroisoquinolin-1-yl) amino) piperidine-1-carboxylate (4.3 g, 69%) as a pale-yellow semi-solid. LC-MS: [M+H⁺]=362.15.

### Step 2:

To a stirred solution of tert-butyl (R)-3-((6-chloroisoquinolin-1-yl) amino) piperidine-1-carboxylate (2.5 g, 6.91 mmol) in THF (20 mL) was added LiHMDS in THF (14.80 mL, 20.73 mmol) at 0° C. and stirred for 30 min. Then 2-fluoro-4-iodobenzoyl chloride (3.93 g, 13.82 mmol) in THF (5 mL) was added to the reaction mixture and stirred at 25° C. for 30 min. The reaction mixture was diluted with water and extracted with EtOAc (2×200 mL). The combined organic layer was washed with brine, dried over Na₂SO₄, and concentrated. The residue was purified by Combi-flash first and then further purified by prep HPLC to afford tert-butyl (R)-3-(N-(6-chloroisoquinolin-1-yl)-2-fluoro-4-iodobenzamido) piperidine-1-carboxylate (700 mg, 46.4%) as an off-white solid. LC-MS: [M+H⁺]=610.1.

### Step 3:

To a stirred solution of tert-butyl (R)-3-(N-(6-chloroisoquinolin-1-yl)-2-fluoro-4-iodobenzamido) piperidine-1-carboxylate (150 mg, 0.246 mmol) in THF (4 mL) were added pyrimidin-2-amine (23 mg, 0.246 mmol) and Cs₂CO₃ (160 mg, 0.492 mmol). The reaction mixture was purged with nitrogen for 10 mins. Then Brettphos Pd G3 (22 mg, 0.025 mmol) was added to reaction mixture and stirred at 100° C. for 16 h. The reaction mixture was diluted with EtOAc (80 mL) and filtered through a celite pad. The filtrate was washed with water, brine solution, dried over Na₂SO₄ and concentrated. The obtained compound was purified by Combi-flash to afford tert-butyl (R)-3-(N-(6-chloroisoquinolin-1-yl)-2-fluoro-4-(pyrimidin-2-ylamino) benzamido) piperidine-1-carboxylate LC-MS: [M+H⁺]=577 and tert-butyl (R)-3-(2-fluoro-4-(pyrimidin-2-ylamino)-N-(6-(pyrimidin-2-ylamino) isoquinolin-1-yl) benzamido) piperidine-1-carboxylate as dark brown solid. LC-MS: [M+H⁺]=636.1.

### Step 4:

To a stirred solution of tert-butyl (R)-3-(N-(6-chloroisoquinolin-1-yl)-2-fluoro-4-(pyrimidin-2-ylamino) benzamido) piperidine-1-carboxylate (100 mg, 0.173 mmol) in DCM (2 mL) was added 4M HCl in 1,4-dioxane (2 mL, 8 mmol) at 0° C. and stirred at room temperature for 3 h. The reaction mixture was concentrated under reduced pressure and dried by lyophilization to afford Example 794 (75 mg, 80%) as a pale yellow solid. LC-MS: [M+H⁺]=477.2. ¹H NMR (CD₃OD, 400 MHZ): δ ppm 8.49-8.46 (m, 3H), 8.10-8.08 (m, 1H), 7.95-7.91 (m, 1H), 7.72-7.71 (m, 1H), 7.65-7.46 (m, 2H), 6.94-6.95 (m, 3H), 5.24-5.19 (m, 1H), 3.90-3.87 (m, 1H), 3.71-3.68 (m, 1H), 3.35-3.48 (m, 1H), 2.87-2.83 (m, 1H), 1.97-1.89 (m, 3H), 1.29-1.26 (m, 1H).

Example 738 isolated as an off-white solid. LC-MS: [M+H⁺]=536.2. ¹H NMR (CD₃OD, 400 MHZ): δ ppm 8.62-8.61 (m, 3H), 8.47 (bs, 2H), 8.34-8.33 (m, 1H), 8.12-8.11 (m, 1H), 7.95 (bs, 1H), 7.84 (bs, 1H), 7.58 (bs, 1H), 7.14-7.04 (m, 3H), 6.91-6.90 (m, 1H), 5.12-5.06 (m, 1H), 3.96-3.94 (m, 1H), 3.67-3.63 (m, 1H), 3.96-3.93 (m, 1H), 2.93-2.90 (m, 1H), 2.03-1.93 (m, 3H), 1.48-1.36 (m, 1H).

### Synthesis of (R)-4-(3H-[1,2,3]triazolo[4,5-b]pyridin-3-yl)-2-fluoro-N-(2-methylthieno[3,2-c]pyridin-4-yl)-N-(piperidin-3-yl)benzamide (Example 739)

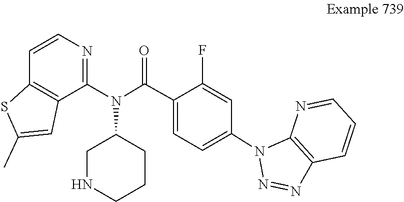 [SMILES: Cc1cc2c(N(C(=O)c3ccc(-n4nnc5cccnc54)cc3F)[C@@H]3CCCNC3)nccc2s1]

Example 739 was prepared according to the procedure described for Example 161 by substituting 4-pyridylboronic acid with methylboronic acid, provided the title compound as an off white solid. LC-MS: [M+H]=488.1.

### Synthesis of (R)-4-(3H-[1,2,3]triazolo[4,5-b]pyridin-3-yl)-N-(2-cyclopropylthieno[3,2-c]pyridin-4-yl)-2-fluoro-N-(piperidin-3-yl)benzamide (Example 740)

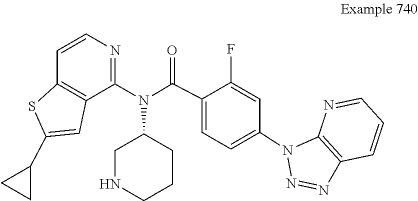 [SMILES: O=C(c1ccc(-n2nnc3cccnc32)cc1F)N(c1nccc2sc(C3CC3)cc12)[C@@H]1CCCNC1]

Example 740 was prepared according to the procedure described for Example 161 by substituting 4-pyridylboronic acid with cyclopropylboronic acid, provided the title compound as off white solid. LC-MS: [M+H]=514.2.

### Synthesis of (R)-2-fluoro-N-(8-methylisoquinolin-1-yl)-4-((4-(morpholinomethyl)pyrimidin-2-yl)amino)-N-(piperidin-3-yl)benzamide (Example 741)

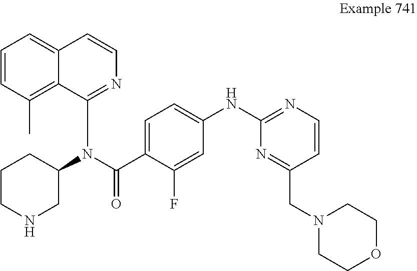 [SMILES: Cc1cccc2ccnc(N(C(=O)c3ccc(Nc4nccc(CN5CCOCC5)n4)cc3F)[C@@H]3CCCNC3)c12]

Example 741 was synthesized following the procedure for Example 686 by substituting tert-butyl piperazine-1-carboxylate with morpholine to afford the title compound as a pale yellow solid. LC-MS: [M+H⁺]=556.3. ¹H NMR (CD₃OD, 400 MHZ): δ ppm 8.50-8.41 (m, 2H), 7.78-7.73 (m, 1H), 7.69-7.75 (m, 1H), 7.54-7.45 (m, 3H), 6.92-6.87 (m, 2H), 6.75-6.71 (m, 1H), 5.09-5.03 (m, 1H), 4.43 (s, 2H), 4.06-3.95 (m, 3H), 3.87-3.81 (m, 3H), 3.63-3.60 (m, 2H), 3.48-3.38 (m, 2H), 3.02-2.98 (m, 2H), 2.83 (s, 3H), 2.68-2.69 (m, 1H), 1.94-1.86 (m, 2H), 1.59-1.52 (m, 1H).

### Synthesis of (R)-2-fluoro-4-((4-((2-methoxyethyl)(methyl)amino)pyrimidin-2-yl)amino)-N-(8-methylisoquinolin-1-yl)-N-(piperidin-3-yl)benzamide (Example 742)

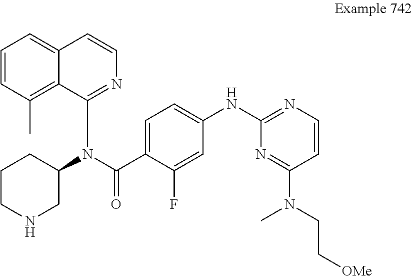 [SMILES: COCCN(C)c1ccnc(Nc2ccc(C(=O)N(c3nccc4cccc(C)c34)[C@@H]3CCCNC3)c(F)c2)n1]

Example 742 was synthesized following the procedure for Example 680 by substituting azetidin-3-ol hydrochloride with 2-methoxy-N-methylethan-1-amine to afford the title compound as a pale-yellow solid. LC-MS: [M+H⁺]=544.3. ¹H NMR (CD₃OD, 400 MHz): δ ppm 8.44-8.40 (m, 1H), 7.78-7.643 (m, 3H), 7.53-7.45 (m, 2H), 7.22-7.16 (m, 1H), 6.83-6.76 (m, 2H), 6.59-6.47 (m, 1H), 5.08-5.02 (m, 1H), 3.98-3.96 (m, 1H), 3.80-3.76 (m, 3H), 3.64-3.62 (m, 1H), 3.55-3.53 (m, 1H), 3.33 (s, 3H), 3.23 (s, 3H), 3.01-2.99 (m, 1H), 2.81 (s, 3H), 2.69-2.67 (m, 1H), 1.95-1.91 (m, 3H), 1.57-1.53 (m, 1H).

### Synthesis of (R)-2-fluoro-4-((4-((2-hydroxyethyl)(methyl)amino)pyrimidin-2-yl)amino)-N-(8-methylisoquinolin-1-yl)-N-(piperidin-3-yl)benzamide (Example 743)

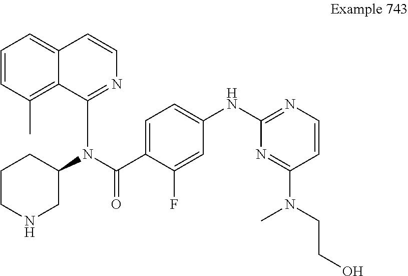 [SMILES: Cc1cccc2ccnc(N(C(=O)c3ccc(Nc4nccc(N(C)CCO)n4)cc3F)[C@@H]3CCCNC3)c12]

Example 743 was synthesized following the procedure for Example 680 by substituting azetidin-3-ol hydrochloride with 2-(methylamino) ethan-1-ol to afford the title compound as an off-white solid. LC-MS: [M+H⁺]=530.30. ¹H NMR (CD₃OD, 400 MHZ): δ ppm 8.54 (bs, 1H), 8.40 (d, J=5.6 Hz, 1H), 7.81 (d, J=6.0 Hz, 1H), 7.71 (d, J=5.6 Hz, 1H), 7.68-7.64 (m, 1H), 7.50-7.46 (m, 1H), 7.43-7.38 (m, 2H), 6.75-6.72 (m, 1H), 6.61-6.57 (m, 1H), 6.13 (d, J=6.0 Hz, 1H), 5.02-4.99 (m, 1H), 3.84-3.81 (m, 1H), 3.71-3.68 (m, 2H), 3.63-3.58 (m, 3H), 3.23-3.19 (m, 1H), 3.09 (s, 3H), 2.83-2.82 (m, 1H), 2.79 (s, 3H), 1.84-1-78 (m, 2H), 1.51-1.47 (m, 1H), 1.37-1.28 (m, 1H).

### Synthesis of (R)-2-fluoro-4-((4-((2-methoxyethyl)amino)pyrimidin-2-yl)amino)-N-(8-methylisoquinolin-1-yl)-N-(piperidin-3-yl)benzamide (Example 744)

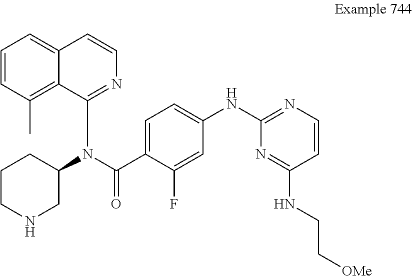 [SMILES: COCCNc1ccnc(Nc2ccc(C(=O)N(c3nccc4cccc(C)c34)[C@@H]3CCCNC3)c(F)c2)n1]

Example 744 was synthesized following the procedure for Example 680 by substituting azetidin-3-ol hydrochloride with 2-methoxyethan-1-amine to afford the title compound as a pale brown solid. LC-MS: [M+H⁺]=530.3. ¹H NMR (CD₃OD, 400 MHZ): δ ppm 8.44-8.40 (m, 1H), 7.77-7.70 (m, 1H), 7.68-7.61 (m, 2H), 7.53-7.45 (m, 2H), 7.26-7.19 (m, 1H), 6.84-6.81 (m, 2H), 6.23 (d, J=7.2 Hz, 1H), 5.08-5.02 (m, 1H), 3.99-3.96 (m, 1H), 3.83-3.77 (m, 1H), 3.56-3.53 (m, 3H), 3.40-3.35 (m, 3H), 3.26-3.15 (m, 1H), 3.00-2.99 (m, 1H), 2.81 (s, 3H), 2.69-2-68 (m, 1H), 1.98-1.88 (m, 3H), 1.57-1.53 (m, 1H), 1.28-1.20 (m, 1H).

### Synthesis of (R)-2-fluoro-4-((4-(methyl(propyl)amino)pyrimidin-2-yl)amino)-N-(8-methylisoquinolin-1-yl)-N-(piperidin-3-yl)benzamide (Example 745)

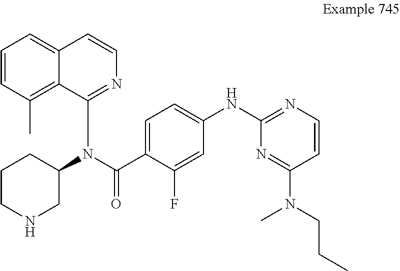 [SMILES: CCCN(C)c1ccnc(Nc2ccc(C(=O)N(c3nccc4cccc(C)c34)[C@@H]3CCCNC3)c(F)c2)n1]

Example 745 was synthesized following the procedure for Example 680 by substituting azetidin-3-ol hydrochloride with N-methylpropan-1-amine to afford the title compound as a brown solid. LC-MS: [M+H⁺]=528.1. ¹H NMR (CD₃OD, 400 MHZ): δ ppm 8.43-8.39 (m, 1H), 7.76-7.63 (m, 3H), 7.52-7.44 (m, 2H), 7.30-7.20 (m, 1H), 6.84-6.75 (m, 2H), 6.55-6.44 (m, 1H), 5.08-5.02 (m, 1H), 3.98-3.96 (m, 1H), 3.83-3.77 (m, 1H), 3.56-3.53 (m, 2H), 3.42-3.39 (m, 1H), 3.20 (s, 3H), 3.01-2.99 (m, 1H), 2.81 (s, 3H), 2.69-2.60 (m, 1H), 1.94-1.88 (m, 2H), 1.71-1.60 (m, 3H), 0.99-0.91 (m, 3H).

### Synthesis of (R)-2-fluoro-N-(8-methylisoquinolin-1-yl)-N-(piperidin-3-yl)-4-((4-(propylamino)pyrimidin-2-yl)amino)benzamide (Example 746)

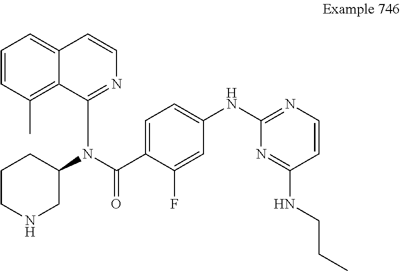 [SMILES: CCCNc1ccnc(Nc2ccc(C(=O)N(c3nccc4cccc(C)c34)[C@@H]3CCCNC3)c(F)c2)n1]

Example 746 was synthesized following the procedure for Example 680 by substituting azetidin-3-ol hydrochloride with propane-1-amine to afford the title product as a yellow solid. LC-MS: [M+H⁺]=514.1. ¹H NMR (CD₃OD, 400 MHZ): δ ppm 8.43-8.40 (m, 1H), 7.78-7.71 (m, 1H), 7.68-7.60 (m, 2H), 7.53-7.45 (m, 2H), 7.34-7.27 (m, 1H), 6.84-6.78 (m, 2H), 6.19 (d, J=7.2 Hz, 1H), 5.08-5.03 (m, 1H), 3.99-3.96 (m, 1H), 3.83-3.78 (m, 1H), 3.63-3.58 (m, 1H), 3.41-3.30 (m, 2H), 3.02-2.99 (m, 1H), 2.81 (s, 3H), 2.69-2.66 (m, 1H), 1.94-1.88 (m, 2H), 1.64-1.57 (m, 3H), 1.01-0.95 (m, 3H).

### Synthesis of (R)-3-methyl-N-(8-methylisoquinolin-1-yl)-N-(piperidin-3-yl)-4-(pyrimidin-2-ylamino)benzamide (Example 747)

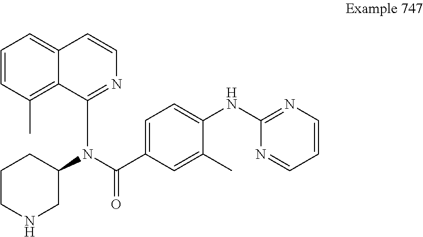 [SMILES: Cc1cc(C(=O)N(c2nccc3cccc(C)c23)[C@@H]2CCCNC2)ccc1Nc1ncccn1]

Example 747 was prepared following the example for 210 by substituting 2-flouro-4-nitrobenzoyl chloride with 3-methyl-4-nitrobenzoyl chloride to afford the title product as an off-white solid. LC-MS: [M+H⁺]=453.1. ¹H NMR (CD₃OD, 400 MHZ): δ ppm 8.55-8.46 (m, 3H), 7.83 (d, J=5.2 Hz, 1H), 7.72-7.69 (m, 1H), 7.52-7.49 (m, 1H), 7.42-7.39 (m, 1H), 7.16-7.12 (m, 1H), 7.09-7.06 (m, 1H), 7.03-7.01 (m, 2H), 5.12-5.03 (m, 1H), 3.96-3.93 (m, 1H), 3.81-3.75 (m, 1H), 3.47-3.37 (m, 1H), 3.00-2.96 (m, 1H), 2.75 (s, 3H), 2.00 (s, 3H), 1.94-1.90 (m, 2H), 1.84-1.81 (m, 1H), 1.45-1.41 (m, 1H).

### Synthesis of (R,E)-2-fluoro-4-((4-(3-hydroxyprop-1-en-1-yl)pyrimidin-2-yl)amino)-N-(8-methylisoquinolin-1-yl)-N-(piperidin-3-yl)benzamide (Example 748)

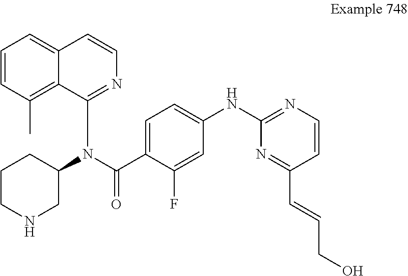 [SMILES: Cc1cccc2ccnc(N(C(=O)c3ccc(Nc4nccc(/C=C/CO)n4)cc3F)[C@@H]3CCCNC3)c12]

Example 748 was synthesized following the procedure for Example 720 by substituting tert-butyl(5-(4,4,5,5-tetramethyl-1,3,2-dioxaborolan-2-yl)pyridin-2-yl)carbamate with (E)-3-(4,4,5,5-tetramethyl-1,3,2-dioxaborolan-2-yl)prop-2-en-1-ol, provided the title compound formic acid salt as a white solid. LC-MS: [M+H⁺]=513.3. ¹H NMR (CD₃OD, 400 MHz): δ ppm 8.53 (s, 1H), 8.43 (d, J=5.2 Hz, 1H), 8.2 (d, J=5.2 Hz, 1H), 7.73 (d, J=5.6 Hz, 1H), 7.67-7.65 (m, 1H), 7.55-7.42 (m, 3H), 7.05-6.99 (m, 1H), 6.85-6.80 (m, 2H), 6.64-6.52 (m, 1H), 5.04-5.01 (m, 1H), 4.31-4.30 (m, 1H), 3.92-3.89 (m, 1H), 3.77-3.70 (m, 1H), 3.59-3.56 (m, 1H), 2.95-2.91 (m, 1H), 2.83 (s, 3H), 2.71-2.67 (m, 1H), 1.92-1.87 (m, 3H), 1.57-1.53 (m, 1H).

### Synthesis of (R,E)-2-fluoro-4-((4-(3-methoxyprop-1-en-1-yl)pyrimidin-2-yl)amino)-N-(8-methylisoquinolin-1-yl)-N-(piperidin-3-yl)benzamide (Example 749)

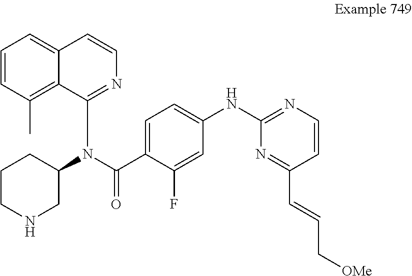 [SMILES: COC/C=C/c1ccnc(Nc2ccc(C(=O)N(c3nccc4cccc(C)c34)[C@@H]3CCCNC3)c(F)c2)n1]

Example 749 was synthesized following the procedure for Example 720 by substituting tert-butyl(5-(4,4,5,5-tetramethyl-1,3,2-dioxaborolan-2-yl)pyridin-2-yl)carbamate with (E)-2-(3-methoxyprop-1-en-1-yl)-4,4,5,5-tetramethyl-1,3,2-dioxaborolane to afford the title product as an off-white solid. LC-MS: [M+H⁺]=527.1. ¹H NMR (CD₃OD, 400 MHZ): δ ppm 8.47-8.40 (m, 1H), 8.32 (d, J=5.2 Hz, 1H), 7.74-7.70 (m, 1H), 7.67-7.62 (m, 1H), 7.59-7.41 (m, 3H), 6.99-6.92 (m, 1H), 6.83-6.80 (m, 2H), 6.74-6.59 (m, 1H), 6.56-6.51 (m, 1H), 5.09-5.02 (m, 1H), 4.17-4.15 (m, 1H), 3.96-3.93 (m, 1H), 3.85-3.79 (m, 1H), 3.48-3.47 (m, 1H), 3.44 (s, 3H), 3.37-3.33 (m, 1H), 3.01-2.99 (m, 1H), 2.80 (s, 3H), 1.98-1.87 (m, 3H), 1.61-1.56 (m, 1H).

### Synthesis of (R)-2-fluoro-N-(8-methylisoquinolin-1-yl)-N-(piperidin-3-yl)-4-((4-(tetrahydro-2H-pyran-4-yl)pyrimidin-2-yl)amino)benzamide (Example 750)

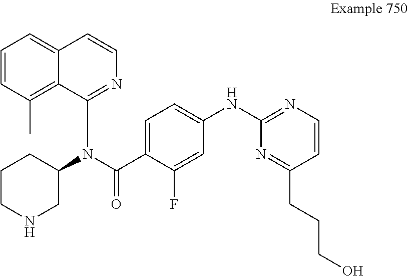 [SMILES: Cc1cccc2ccnc(N(C(=O)c3ccc(Nc4nccc(CCCO)n4)cc3F)[C@@H]3CCCNC3)c12]

Example 750 was synthesized following the procedure for Example 748 to afford the title compound as an off-white solid. LC-MS: [M+H⁺]=515.2. ¹H NMR (CD₃OD, 400 MHz): δ ppm 8.43-8.39 (m, 1H), 8.27-8.24 (m, 1H), 7.73-7.69 (m, 1H), 7.67-7.62 (m, 1H), 7.55-7.42 (m, 3H), 6.83-6.81 (m, 1H), 6.73 (d, J=5.2 Hz, 1H), 6.63-6.59 (m, 1H), 5.06-5.00 (m, 1H), 3.93-3.91 (m, 1H), 3.81-3.75 (m, 1H), 3.59-3.56 (m, 2H), 3.37-3.34 (m, 1H), 3.01-2.93 (m, 2H), 2.79 (s, 3H), 2.72-2.69 (m, 2H), 1.94-1.88 (m, 4H), 1.58-1.54 (m, 1H).

### Synthesis of (R)-2-fluoro-4-((4-(3-methoxyprop-1-yn-1-yl)pyrimidin-2-yl)amino)-N-(8-methylisoquinolin-1-yl)-N-(piperidin-3-yl)benzamide (Example 751)

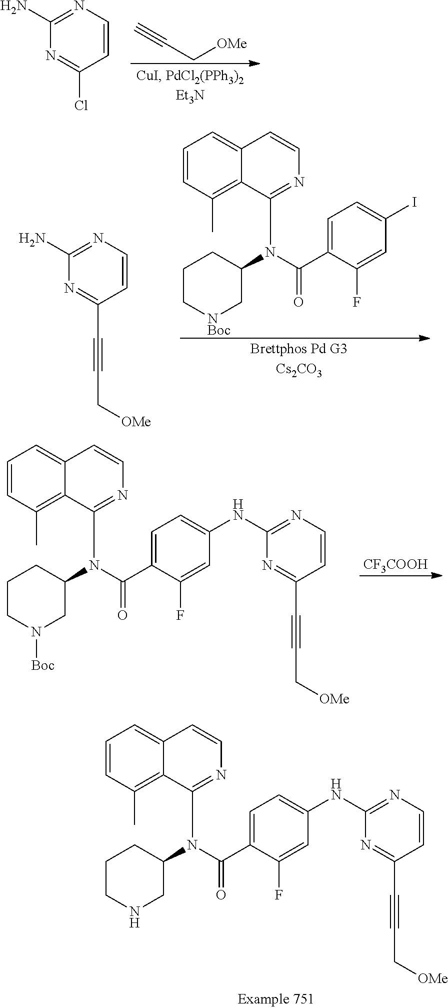

### Step 1:

To a stirred solution of 4-chloropyrimidin-2-amine (500 mg, 3.86 mmol) in DMF (5 mL) were added Et₃N (1.614 mL, 11.58 mmol), 3-methoxyprop-1-yne (541 mg, 7.72 mmol), and CuI (73.5 mg, 0.386 mmol), and the reaction was purged with nitrogen for 10 minutes. Then, PdCl₂ (PPh₃) 2 (282 mg, 0.386 mmol) was added to the reaction mixture, and the reactions was stirred at room temperature for 16 h. The reaction mixture was diluted with EtOAc (40 mL) and filtered through a celite pad. The filtrate was washed with water, brine, dried over Na₂SO₄, and concentrated under reduced pressure. The obtained compound was purified by Combi-Flash using 100% EtOAc in hexane to afford 4-(3-methoxyprop-1-yn-1-yl)pyrimidin-2-amine (500 mg, 63.5%) as an off-white solid. LC-MS: [M+H⁺]=164.1.

Steps 2 and 3 were completed following the procedure from Example 679 by substituting 4-(2-aminopyrimidin-4-yl)cyclohexan-1-ol with 4-(3-methoxyprop-1-yn-1-yl) pyrimidin-2-amine to afford the title compound as a light brown solid. LC-MS: [M+H⁺]=525.2. ¹H NMR (CD₃OD, 400 MHZ): δ 8.43-8.38 (m, 2H), 7.74-7.65 (m, 2H), 7.52-7.42 (m, 3H), 6.80 (s, J=4.8 Hz, 1H), 6.84-6.81 (m, 1H), 6.64-6.60 (m, 1H), 5.09-5.02 (m, 1H), 4.35 (s, 2H), 3.94-3.93 (m, 1H), 3.84-3.78 (m, 1H), 3.46-3.44 (m, 1H), 3.37-3.36 (m, 2H), 3.00-2.99 (m, 2H), 2.83 (s, 3H), 1.95-1.90 (m, 3H), 1.60-1.59 (m, 1H).

### Synthesis of (R)-2-fluoro-4-((4-(3-methoxypropyl)pyrimidin-2-yl)amino)-N-(8-methylisoquinolin-1-yl)-N-(piperidin-3-yl)benzamide (Example 752)

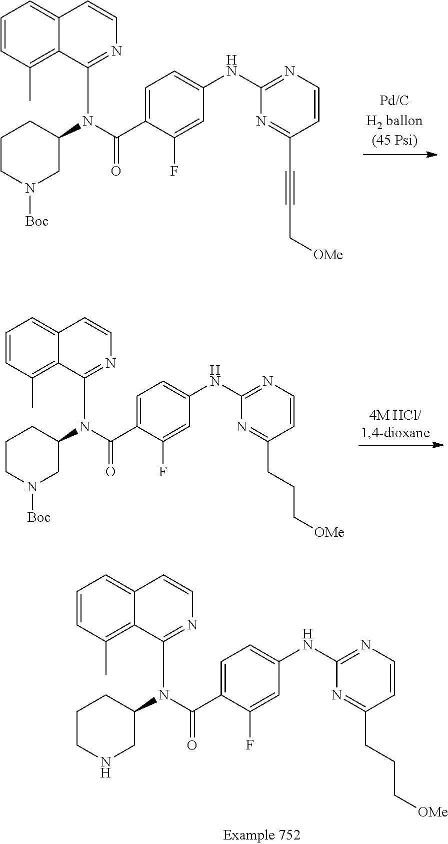 [SMILES: COCC#Cc1ccnc(Nc2ccc(C(=O)N(c3nccc4cccc(C)c34)[C@@H]3CCCN(C(=O)OC(C)(C)C)C3)c(F)c2)n1 ; COCCCc1ccnc(Nc2ccc(C(=O)N(c3nccc4cccc(C)c34)[C@@H]3CCCN(C(=O)OC(C)(C)C)C3)c(F)c2)n1 ; COCCCc1ccnc(Nc2ccc(C(=O)N(c3nccc4cccc(C)c34)[C@@H]3CCCNC3)c(F)c2)n1]

### Step 1:

To a stirred solution of tert-butyl (R)-3-(2-fluoro-4-((4-(3-methoxyprop-1-yn-1-yl) pyrimidin-2-yl)amino)-N-(8-methylisoquinolin1-yl) benzamido) piperidine-1-carboxylate (20 mg, 0.032 mmol) in ethanol (5 mL), was added Pd/C (3.41 mg, 0.032 mmol), and the reaction was stirred under a hydrogen balloon at room temperature for 5 h. The reaction mixture was diluted with ethanol and DCM (20 mL) and filtered through celite. The reaction mixture was concentrated and then was purified by prep HPLC (Conditions: X-SELECT CSH, Mobile phase A: 0.1% formic acid in H2O, Mobile phase B: MeCN (100%), T/% B: 0/60, 10/80, MeCN+H₂O+THF). Pure fractions were concentrated under reduced pressure to afford tert-butyl (R)-3-(2-fluoro-4-((4-(3-methoxypropyl)pyrimidin-2-yl) amino)-N-(8-methylisoquinolin-1-yl)benzamido)piperidine-1-carboxylate (55 mg, 68.1%) as a white solid. LC-MS: [M+H⁺]=629.3.

### Step 2:

To a solution of tert-butyl (R)-3-(2-fluoro-4-((4-(3-methoxypropyl)pyrimidin-2-yl) amino)-N-(8-methylisoquinolin-1-yl) benzamido) piperidine-1-carboxylate (55 mg, 0.087 mmol) in DCM (2 mL) was added 4M HCl in 1,4-dioxane (2 mL) at 0° C., and the reaction was stirred at room temperature for 2 h. The reaction mixture was concentrated under reduced pressure, washed with diethyl ether, and dried by lyophilization to afford the title compound as a yellow solid. LC-MS: [M+H⁺]=529.30. ¹H NMR (CD₃OD, 400 MHZ): δ 8.44 (d, J=5.6 Hz, 1H), 8.33 (d, J=6 Hz, 1H), 7.75 (d, J=5.6 Hz, 1H), 7.68-7.63 (m, 1H), 7.53-7.46 (m, 2H), 7.35-7.27 (m, 1H), 7.06 (d, J=6.4 Hz, 1H), 6.89-6.81 (m, 2H), 5.09-5.03 (m, 1H), 3.99-3.97 (m, 1H), 3.84-3.81 (m, 1H), 3.45-3.34 (m, 4H), 3.02-2.96 (m, 1H), 2.90-2.82 (m, 3H), 2.82 (s, 3H), 2.71-2.68 (m, 1H), 1.98-1.91 (m, 5H), 1.60-1.31 (m, 1H).

### Synthesis of (R)-4-((4-ethyl-5-fluoropyrimidin-2-yl)amino)-2-fluoro-N-(8-methylisoquinolin-1-yl)-N-(piperidin-3-yl)benzamide (Example 753)

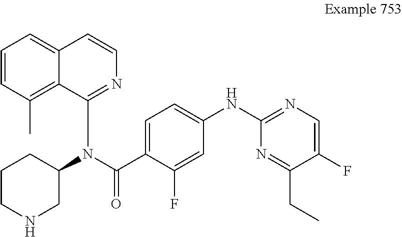 [SMILES: CCc1nc(Nc2ccc(C(=O)N(c3nccc4cccc(C)c34)[C@@H]3CCCNC3)c(F)c2)ncc1F]

Example 753 was synthesized following the procedure for Example 210 by substituting 2-chloropyrimidine with 2-chloro-4-ethyl-5-fluoropyrimidine to afford the title compound as a pale-yellow solid. LC-MS: [M+H⁺]=503.2. ¹H NMR (CD₃OD, 400 MHZ): δ 8.44-8.40 (m, 1H), 8.20-8.19 (m, 1H), 7.76-7.72 (m, 1H), 7.68-7.64 (m, 1H), 7.52-7.45 (m, 3H), 6.81-6.78 (m, 1H), 6.64-6.60 (m, 1H), 5.08-5.03 (m, 1H), 3.96-3.93 (m, 1H), 3.84-3.79 (m, 1H), 3.47-3.31 (m, 1H), 3.01-2.98 (m, 1H), 2.83 (s, 3H), 2.75-2.72 (m, 2H), 1.98-1.86 (m, 2H), 1.59-1.55 (m, 2H), 1.28-1.23 (m, 3H).

### Synthesis of (R)-2-fluoro-4-((5-fluoro-4-methylpyrimidin-2-yl)amino)-N-(8-methylisoquinolin-1-yl)-N-(piperidin-3-yl)benzamide (Example 754)

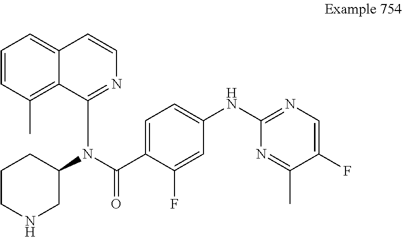 [SMILES: Cc1nc(Nc2ccc(C(=O)N(c3nccc4cccc(C)c34)[C@@H]3CCCNC3)c(F)c2)ncc1F]

Example 754 was synthesized following the procedure for example E10 by substituting 2-chloropyrimidine with 2-chloro-4-methyl-5-fluoropyrimidine to afford the title compound as an off-white solid. LC-MS: [M+H⁺]=489.2. ¹H NMR (CD₃OD, 400 MHZ): δ 8.43-8.39 (m, 1H), 8.18-8.13 (m, 1H), 7.73-7.65 (m, 2H), 7.50-7.42 (m, 3H), 6.79-6.76 (m, 1H), 6.62-6.58 (m, 1H), 5.04-5.01 (m, 1H), 3.92-3.88 (m, 1H), 3.77-3.71 (m, 1H), 3.48-3.47 (m, 1H), 2.92-2.88 (m, 1H), 2.82 (s, 3H), 2.37 (s, 3H), 1.90-1.89 (m, 3H), 1.56-1.51 (m, 1H).

### Synthesis of (R)—N-(6-chloro-8-methylisoquinolin-1-yl)-4-(5-methyl-1,3,4-thiadiazol-2-yl)-N-(piperidin-3-yl)benzamide (Example 755)

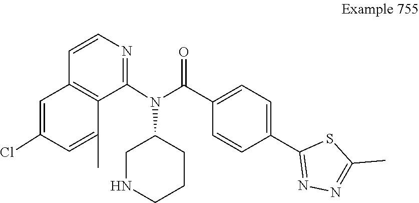 [SMILES: Cc1nnc(-c2ccc(C(=O)N(c3nccc4cc(Cl)cc(C)c34)[C@@H]3CCCNC3)cc2)s1]

Example 755 was prepared according to the procedure described for Example 816 by substituting 4-bromo-2-chloro-benzoyl chloride and 4-bromo-1-methyl-triazole with 4-bromo-benzoyl chloride and 2-bromo-5-methyl-1,3,4-thiadiazole, providing the title compound as a white solid. LC-MS: [M+H⁺]=478.2. ¹H NMR (400 MHZ, DMSO-d₆): δ 8.58-8.51 (m, 1H), 7.89 (d, J=1.8 Hz, 1H), 7.81 (d, J=5.6 Hz, 1H), 7.56 (d, J=8.3 Hz, 2H), 7.50-7.45 (m, 1H), 7.16-7.08 (m, 2H), 4.60-4.45 (m, 1H), 3.12 (br t, J=10.9 Hz, 1H), 2.90-2.80 (m, 1H), 2.73-2.64 (m, 7H), 2.33 (br d, J=1.5 Hz, 1H), 1.59-1.45 (m, 3H), 1.14-1.00 (m, 1H).

### Synthesis of (R)-4-(1H-benzo[d]imidazol-1-yl)-2-fluoro-N-(2-methylthieno[3,2-c]pyridin-4-yl)-N-(piperidin-3-yl)benzamide (Example 756)

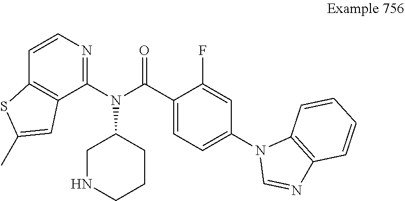 [SMILES: Cc1cc2c(N(C(=O)c3ccc(-n4cnc5ccccc54)cc3F)[C@@H]3CCCNC3)nccc2s1]

Example 756 was prepared according to the procedure described for Example 161 by substituting 4-pyridylboronic acid and tert-butyl (R)-3-((6-chloro-8-methylisoquinolin-1-yl) amino) piperidine-1-carboxylate with methylboronic acid and tert-butyl (R)-3-(4-(1H-benzo[d]imidazol-1-yl)-N-(2-bromothieno[3,2-c]pyridin-4-yl)-2-fluorobenzamido)piperidine-1-carboxylate, provided the title compound as an off white solid. LC-MS: [M+H⁺]=486.3. ¹H NMR (400 MHZ, DMSO-d₆): δ 9.55-9.04 (m, 3H), 8.41-8.24 (m, 1H), 8.18-7.88 (m, 2H), 7.82 (br d, J=2.1 Hz, 1H), 7.63-7.31 (m, 7H), 5.12-4.78 (m, 1H), 3.70-3.64 (m, 1H), 3.34-3.10 (m, 2H), 2.74-2.56 (m, 4H), 2.03-1.91 (m, 1H), 1.89-1.75 (m, 2H), 1.23 (s, 1H).

### Synthesis of 2-methyl-4-(5-methyl-1,3,4-thiadiazol-2-yl)-N-(3-methylthieno[3,2-c]pyridin-4-yl)-N-[(3R)-3-piperidyl]benzamide (Example 757)

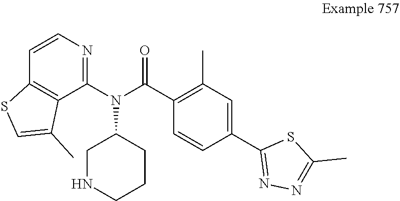 [SMILES: Cc1nnc(-c2ccc(C(=O)N(c3nccc4scc(C)c34)[C@@H]3CCCNC3)c(C)c2)s1]

Example 757 was prepared according to the procedure described for Example 633 by substituting tert-butyl (R)-3-(N-(3-methylthieno[3,2-c]pyridin-4-yl)-4-(4,4,5,5-tetramethyl-1,3,2-dioxaborolan-2-yl)benzamido)piperidine-1-carboxylate with tert-butyl (3R)-3-[[2-methyl-4-(4,4,5,5-tetramethyl-1,3,2-dioxaborolan-2-yl)benzoyl]-(3-methylthieno[3,2-c]pyridin-4yl)amino]piperidine-1-carboxylate and 2-bromo-5-(2-methoxyethyl)-1,3,4-thiadiazole with 2-bromo-5-methyl-1,3,4-thiadiazole to afford the title compound as an off-white solid. LC-MS: [M+H⁺]=464.2. ¹H NMR (CD₃OD, 400 MHZ): δ 1.15-1.31 (m, 1H) 1.81-1.99 (m, 3H) 2.48-2.55 (m, 3H) 2.70 (s, 3H) 2.76 (s, 3H) 2.85-2.96 (m, 1H) 3.37 (br d, J=12.76 Hz, 1H) 3.64 (br t, J=11.44 Hz, 1H) 4.08 (br dd, J=11.76, 3.25 Hz, 1H) 4.97-5.07 (m, 1H) 7.05 (d, J=8.00 Hz, 1H) 7.32 (dd, J=8.00, 1.13 Hz, 1H) 7.51-7.60 (m, 2H) 7.85 (d, J=5.38 Hz, 1H) 8.23 (d, J=5.38 Hz, 1H).

### Synthesis of (R)-2-chloro-4-(1-methyl-1H-1,2,3-triazol-4-yl)-N-(3-methylthieno[3,2-c]pyridin-4-yl)-N-(piperidin-3-yl)benzamide (Example 758)

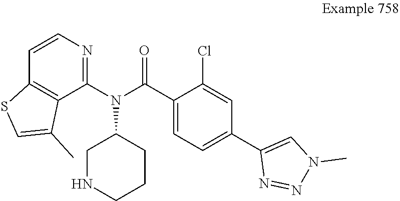 [SMILES: Cc1csc2ccnc(N(C(=O)c3ccc(-c4cn(C)nn4)cc3Cl)[C@@H]3CCCNC3)c12]

Example 758 was prepared according to the procedure described for Example 816 by substituting tert-butyl (3R)-3-[(6-chloro-8-methyl-1-isoquinolyl) amino] piperidine-1-carboxylate with tert-butyl (R)-3-((3-methylthieno[3,2-c]pyridin-4-yl)amino)piperidine-1-carboxylate, providing the title compound as a light yellow solid. LC-MS: [M+H⁺]=467.2. ¹H NMR (400 MHZ, DMSO-d₆): δ 8.83-8.70 (m, 1H), 8.53-8.45 (m, 1H), 8.32-8.23 (m, 1H), 8.02-7.94 (m, 1H), 7.71-7.60 (m, 2H), 7.44-7.31 (m, 1H), 7.06-6.93 (m, 1H), 4.91-4.76 (m, 1H), 4.20-3.98 (m, 4H), 3.23-3.15 (m, 1H), 2.67 (s, 2H), 2.62 (s, 3H), 1.83-1.64 (m, 3H), 0.87-0.75 (m, 1H).

### Synthesis of (R)-2-fluoro-N-(5-fluoroisoquinolin-1-yl)-4-(1-methyl-1H-1,2,3-triazol-4-yl)-N-(piperidin-3-yl)benzamide (Example 759)

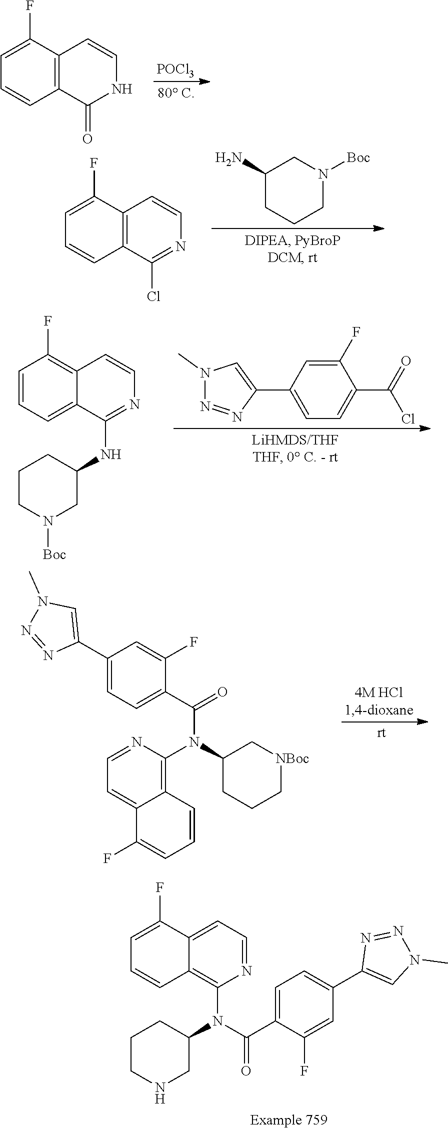

### Step 1:

A solution of 5-fluoro-3,8a-dihydroisoquinolin-1(2H)-one (400 mg, 2.422 mmol) in POCl₃ (0.6 mL, 72.7 mmol) was stirred at 80° C. for 3 h. The reaction was concentrated and diluted with CH₂Cl₂ (100 mL). The organic layer was washed with saturated NaHCO₃ (50 mL), brine, dried over Na₂SO₄, and concentrated to afford 1-chloro-5-fluoroisoquinoline (430 mg, 98%) as an off-white solid. LC-MS: [M+H⁺]=182.1.

### Step 2:

To a stirred solution of 1-chloro-5-fluoroisoquinoline (400 mg, 2.203 mmol) and tert-butyl (R)-3-aminopiperidine-1-carboxylate (441 mg, 2.203 mmol) in CH₂Cl₂ (20 mL) were added DIPEA (1.54 mL, 8.81 mmol) and PyBroP (1.54 g, 3.30 mmol), and the reaction was stirred at room temperature for 16 h. The reaction mixture was diluted with water (50 mL) and extracted with EtOAc (2×50 mL). The combined organic layer was washed with brine, dried over Na₂SO₄, and concentrated. The residue was purified by Combi-Flash using 20-30% EtOAc in hexane to afford tert-butyl (R)-3-((5-fluoroisoquinolin-1-yl)amino)piperidine-1-carboxylate (500 mg, 62%) as an off-white solid. LC-MS: [M+H⁺]=346.2.

### Steps 3-4:

Example 759 was prepared according to the Boc removal procedure described for Example 96, provided the title compound as a white solid. LC-MS: [M+H⁺]=449.2. ¹H NMR (CD₃OD, 400 MHZ): δ 8.53 (d, J=5.6 Hz, 1H), 8.16 (s, 1H), 7.96-7.90 (m, 2H), 7.46-7.43 (m, 1H), 7.28-7.25 (m, 1H), 7.21-7.19 (m, 2H), 7.08-7.06 (m, 1H), 5.28-5.22 (m, 1H), 4.06 (s, 3H), 3.94-3.91 (m, 1H), 3.69-3.67 (m, 1H), 3.31-3.30 (m, 1H), 2.87-2.85 (m, 1H), 1.97-1.98-1.89 (m, 3H), 1.28-1.24 (m, 1H).

### Synthesis of (R)-2-fluoro-N-(5-fluoro-8-methylisoquinolin-1-yl)-4-(5-methyl-1,3,4-thiadiazol-2-yl)-N-(piperidin-3-yl)benzamide (Example 760)

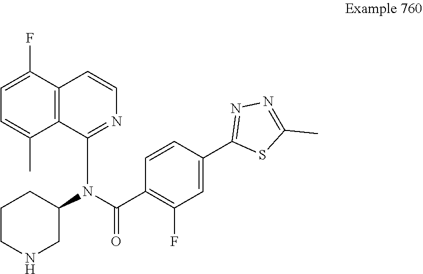 [SMILES: Cc1nnc(-c2ccc(C(=O)N(c3nccc4c(F)ccc(C)c34)[C@@H]3CCCNC3)c(F)c2)s1]

Example 760 was made following the procedure for Example 761 by substituting 4-bromo-1-methyl-1H-1,2,3-triazole with 2-bromo-5-methyl-1,3,4-thiadiazole to afford the title compound (57 mg, 91%) as an off-white solid. LC-MS: [M+H⁺]=480.8. ¹H NMR (CD₃OD, 400 MHz): δ 8.54-8.51 (m, 1H), 8.18 (s, 1H), 7.93-7.89 (m, 1H), 7.47-7.37 (m, 2H), 7.30-7.24 (m, 2H), 7.00-6.95 (m, 1H), 5.11-5.04 (m, 1H), 4.02-3.99 (m, 1H), 3.84-3.78 (m, 1H), 3.45-3.39 (m, 1H), 3.03-2.99 (m, 1H), 2.81 (s, 3H), 2.75 (s, 3H), 2.63-2.61 (m, 1H), 1.97-1.95 (m, 2H), 1.62-1.53 (m, 1H).

### Synthesis of (R)-2-fluoro-N-(5-fluoro-8-methylisoquinolin-1-yl)-4-(1-methyl-1H-1,2,3-triazol-4-yl)-N-(piperidin-3-yl)benzamide (Example 761)

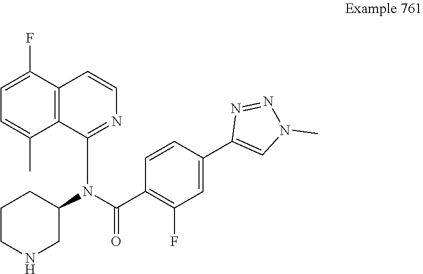 [SMILES: Cc1ccc(F)c2ccnc(N(C(=O)c3ccc(-c4cn(C)nn4)cc3F)[C@@H]3CCCNC3)c12]

Example 761 was prepared according to the procedure described for Example 474 by substituting 6-fluoro-2-methylbenzaldehyde with 5-fluoro-2-methylbenzaldehyde, provided the title compound hydrochloride salt as an off-white solid. LC-MS: [M+H⁺]=463.75. ¹H NMR (CD₃OD, 400 MHZ): δ 8.54-8.50 (m, 1H), 8.18 (s, 1H), 7.91-7.90 (m, 1H), 7.42-7.41 (m, 1H), 7.27-7.25 (m, 3H), 6.87-6.83 (m, 1H), 5.07-5.04 (m, 1H), 4.2 (s, 3H), 4.07-3.91 (m, 1H), 3.82-3.79 (m, 1H), 3.31-3.29 (m, 1H), 3.00-2.99 (m, 1H), 2.82 (s, 3H), 2.79-2.70 (m, 1H), 1.97-1.96 (m, 2H), 1.61-1.58 (m, 1H).

### Synthesis of (R)-2-fluoro-N-(6-methoxyisoquinolin-1-yl)-4-(5-methyl-1,3,4-thiadiazol-2-yl)-N-(piperidin-3-yl)benzamide (Example 762)

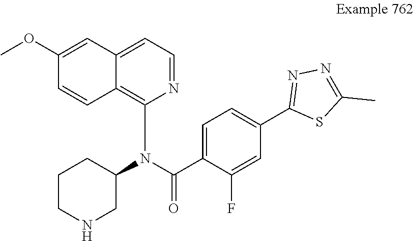

Example 762 was prepared according to the procedure described for Example 127 by substituting tert-butyl (R)-3-((8-methylisoquinolin-1-yl)amino)piperidine-1-carboxylate with tert-butyl (R)-3-((6-methoxyisoquinolin-1-yl)amino)piperidine-1-carboxylate, provided the title compound hydrochloride salt as an off-white solid. LC-MS: [M+H⁺]=478.1. ¹H NMR (CD₃OD, 400 MHZ): δ 8.31 (d, J=6 Hz, 1H), 8.05-8.02 (m, 1H), 7.65 (d, J=6 Hz, 1H), 7.46-7.43 (m, 1H), 7.34-7.29 (m, 2H), 7.23-7.16 (m, 2H), 5.20-5.02 (m, 1H), 3.92-3.90 (m, 1H), 3.88 (s, 3H), 3.67-3.64 (m, 1H), 2.86-2.84 (m, 1H), 2.73 (s, 3H), 1.98-1.85 (m, 3H), 1.39-1.19 (m, 2H).

### Synthesis of (R)—N-(6-chloro-8-methylisoquinolin-1-yl)-4-(1,5-dimethyl-1H-1,2,3-triazol-4-yl)-2-fluoro-N-(piperidin-3-yl)benzamide (Example 763)

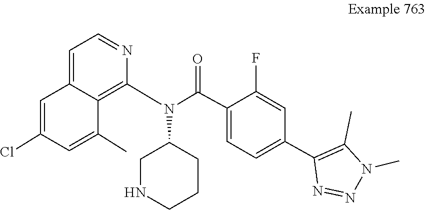 [SMILES: Cc1cc(Cl)cc2ccnc(N(C(=O)c3ccc(-c4nnn(C)c4C)cc3F)[C@@H]3CCCNC3)c12]

Example 763 was prepared according to the procedure described for Example 816 by substituting 4-bromo-2-chloro-benzoyl chloride and 2-bromo-5-methyl-1, 3, 4-thiadiazole with 4-bromo-2-fluoro-benzoyl chloride and 4-bromo-1, 5-dimethyl-triazole, providing the title compound as a white solid. LC-MS: [M+H⁺]=493.3. ¹H NMR (400 MHZ, CD₃OD): δ 8.49-8.39 (m, 1H), 7.74-7.63 (m, 2H), 7.49-7.41 (m, 1H), 7.16-7.07 (m, 1H), 7.06-7.00 (m, 1H), 6.98-6.88 (m, 1H), 5.17-5.02 (m, 1H), 4.01 (s, 4H), 3.89-3.78 (m, 1H), 3.42 (br d, J=12.4 Hz, 1H), 3.08-2.96 (m, 1H), 2.88-2.81 (m, 3H), 2.34 (s, 3H), 2.06-1.83 (m, 3H), 1.73-1.54 (m, 1H).

### Synthesis of (R)-2-chloro-N-(6-chloro-8-methylisoquinolin-1-yl)-4-(5-methyl-1,3,4-oxadiazol-2-yl)-N-(piperidin-3-yl)benzamide (Example 764)

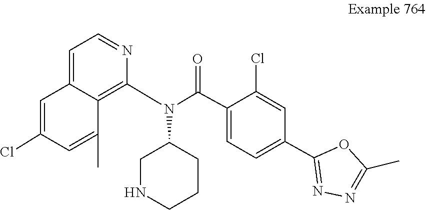 [SMILES: Cc1nnc(-c2ccc(C(=O)N(c3nccc4cc(Cl)cc(C)c34)[C@@H]3CCCNC3)c(Cl)c2)o1]

Example 764 was prepared according to the procedure described for Example 816 by substituting 2-bromo-5-methyl-1, 3, 4-thiadiazole with 2-bromo-5-methyl-1,3,4-oxadiazole, providing the title compound as a yellow solid. LC-MS: [M+H⁺]=496.2. ¹H NMR (400 MHz, CD₃OD): δ 8.18 (d, J=3.8 Hz, 1H), 8.10 (d, J=8.1 Hz, 1H), 7.87 (d, J=1.8 Hz, 1H), 7.71-7.54 (m, 3H), 7.27 (d, J=7.0 Hz, 1H), 4.89 (br s, 1H), 4.50-4.37 (m, 1H), 4.29-4.17 (m, 1H), 3.91-3.70 (m, 1H), 3.63-3.35 (m, 2H), 3.09-2.96 (m, 3H), 2.69-2.61 (m, 3H), 2.37-2.23 (m, 1H), 2.06-1.95 (m, 1H), 1.93-1.67 (m, 1H).

### Synthesis of (R)—N-(6-chloro-8-methylisoquinolin-1-yl)-2-fluoro-4-(5-methyl-1,3,4-oxadiazol-2-yl)-N-(piperidin-3-yl)benzamide (Example 765)

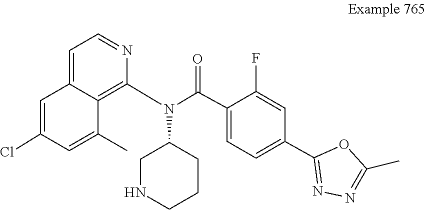 [SMILES: Cc1nnc(-c2ccc(C(=O)N(c3nccc4cc(Cl)cc(C)c34)[C@@H]3CCCNC3)c(F)c2)o1]

Example 765 was prepared according to the procedure described for Example 816 by substituting 4-bromo-2-chloro-benzoyl chloride and 2-bromo-5-methyl-1, 3, 4-thiadiazole with 4-bromo-2-fluoro-benzoyl chloride and 2-bromo-5-methyl-1, 3, 4-oxadiazole, providing the title compound as a white solid. LC-MS: [M+H⁺]=480.3. ¹H NMR (400 MHZ, CD₃OD): δ 8.45-8.36 (m, 1H), 7.72-7.66 (m, 1H), 7.65-7.60 (m, 1H), 7.48 (s, 1H), 7.44 (dd, J=1.4, 10.4 Hz, 1H), 7.38 (dd, J=1.5, 8.1 Hz, 1H), 7.10-7.00 (m, 1H), 4.77-4.66 (m, 1H), 3.73 (br d, J=12.0 Hz, 1H), 3.39-3.33 (m, 1H), 3.04-2.98 (m, 1H), 2.88-2.82 (m, 3H), 2.75-2.61 (m, 1H), 2.53 (s, 3H), 1.82-1.63 (m, 3H), 1.46-1.37 (m, 1H).

### Synthesis of (R)—N-(6-chloro-8-methylisoquinolin-1-yl)-2-fluoro-4-(5-(methyl-d3)-1,3,4-thiadiazol-2-yl)-N-(piperidin-3-yl)benzamide (Example 766)

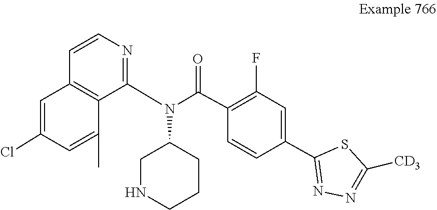 [SMILES: [2H]C([2H])([2H])c1nnc(-c2ccc(C(=O)N(c3nccc4cc(Cl)cc(C)c34)[C@@H]3CCCNC3)c(F)c2)s1]

Example 766 was prepared according to the procedure described for Example 816 by substituting 4-bromo-2-chloro-benzoyl chloride and 2-bromo-5-methyl-1, 3, 4-thiadiazole with 4-bromo-2-fluoro-benzoyl chloride and 2-bromo-5-(trideuteriomethyl)-1,3,4-thiadiazole, providing the title compound as a white solid. LC-MS: [M+H⁺]=499.3. ¹H NMR (400 MHZ, CD₃OD): δ 8.47-8.37 (m, 1H), 7.69 (s, 1H), 7.66-7.59 (m, 1H), 7.48 (s, 1H), 7.43 (dd, J=1.4, 10.6 Hz, 1H), 7.29 (dd, J=1.6, 8.1 Hz, 1H), 7.05-6.97 (m, 1H), 4.71 (br d, J=10.0 Hz, 1H), 3.73 (br d, J=12.1 Hz, 1H), 3.39-3.33 (m, 1H), 3.05-2.98 (m, 1H), 2.91-2.80 (m, 3H), 2.60-2.46 (m, 1H), 1.96-1.63 (m, 3H), 1.37 (br dd, J=7.2, 9.4 Hz, 1H).

### Synthesis of 2-fluoro-N-(6-fluoro-8-methyl-1-isoquinolyl)-N-[(3R)-3-piperidyl]-4-[5-(trideuteriomethyl)-1,3,4-thiadiazol-2-yl]benzamide (Example 767)

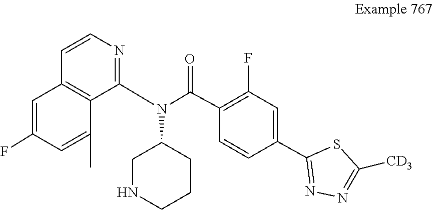 [SMILES: [2H]C([2H])([2H])c1nnc(-c2ccc(C(=O)N(c3nccc4cc(F)cc(C)c34)[C@@H]3CCCNC3)c(F)c2)s1]

Example 767 was synthesized following the procedure for Example 833 by substituting 2-bromo-5-methyl-1,3,4-thiadiazole with 2-bromo-5-(trideuteriomethyl)-1,3,4-thiadiazole to afford the title compound as a white solid. LC-MS: [M+H⁺]=483.3. ¹H NMR (400 MHZ, DMSO-d₆): δ 9.14-8.90 (m, 1H), 8.56-8.41 (m, 1H), 8.10-7.78 (m, 1H), 7.59-7.47 (m, 2H), 7.38-7.23 (m, 1H), 7.13 (s, 1H), 7.15-6.92 (m, 1H), 4.97-4.68 (m, 1H), 3.85-3.77 (m, 1H), 3.60-3.49 (m, 1H), 3.29-3.15 (m, 1H), 2.96 (s, 1H), 2.99-2.79 (m, 3H), 2.04-1.90 (m, 1H), 1.87-1.73 (m, 2H), 1.38-1.21 (m, 1H).

### Synthesis of (R)-4-(5-chloropyridazin-3-yl)-2-fluoro-N-(3-methylthieno[3,2-c]pyridin-4-yl)-N-(piperidin-3-yl)benzamide (Example 768)

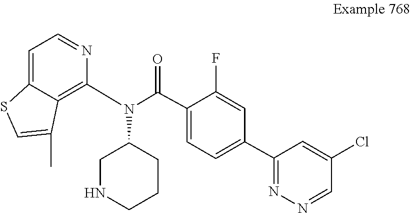 [SMILES: Cc1csc2ccnc(N(C(=O)c3ccc(-c4cc(Cl)cnn4)cc3F)[C@@H]3CCCNC3)c12]

Example 768 was prepared according to the procedure described for Example 699 by substituting 2-bromo-5-methyl-1,3,4-oxadiazole with 3,5-dichloropyridazine, providing the title compound as a white solid. LC-MS: [M+H⁺]=482.2. ¹H NMR (400 MHZ, DMSO-d₆): δ 9.57-9.29 (m, 1H), 8.75-8.39 (m, 1H), 8.38-8.10 (m, 2H), 8.04-7.88 (m, 1H), 7.86-7.73 (m, 1H), 7.71-7.52 (m, 2H), 7.22-7.06 (m, 1H), 4.63-4.35 (m, 1H), 3.13-2.96 (m, 1H), 2.90-2.75 (m, 1H), 2.64 (s, 1H), 2.58-2.53 (m, 3H), 2.37-2.23 (m, 1H), 2.15-1.90 (m, 1H), 1.84-1.46 (m, 3H), 0.95-0.54 (m, 1H).

### Synthesis of (R)—N-(6-chloro-8-methylisoquinolin-1-yl)-5-(5-chloropyridazin-3-yl)-N-(piperidin-3-yl)picolinamide (Example 769)

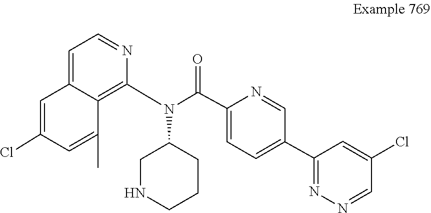 [SMILES: Cc1cc(Cl)cc2ccnc(N(C(=O)c3ccc(-c4cc(Cl)cnn4)cn3)[C@@H]3CCCNC3)c12]

Example 769 was prepared according to the procedure described for Example 816 by substituting 4-bromo-2-chloro-benzoyl chloride and 2-bromo-5-methyl-1,3,4-thiadiazole with 5-bromopyridine-2-carbonyl chloride and 3,5-dichloropyridazine, providing the title compound as a white solid. LC-MS: [M+H⁺]=493.3. ¹H NMR (400 MHZ, CD₃OD): δ 9.27 (d, J=2.1 Hz, 1H), 8.62-8.55 (m, 1H), 8.53-8.48 (m, 1H), 8.39-8.25 (m, 2H), 8.10-8.02 (m, 1H), 7.94 (d, J=1.9 Hz, 1H), 7.91-7.83 (m, 1H), 7.61-7.53 (m, 1H), 5.06-4.93 (m, 1H), 4.02 (br d, J=11.8 Hz, 1H), 3.53 (t, J=11.4 Hz, 1H), 3.35 (br s, 1H), 2.95-2.80 (m, 4H), 2.01-1.72 (m, 3H), 1.18-1.04 (m, 1H).

### Synthesis of (R)-4-(1,5-dimethyl-1H-1,2,3-triazol-4-yl)-N-(6-fluoro-8-methylisoquinolin-1-yl)-N-(piperidin-3-yl)benzamide (Example 770)

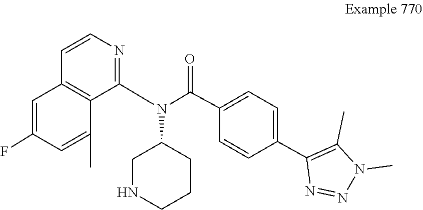 [SMILES: Cc1cc(F)cc2ccnc(N(C(=O)c3ccc(-c4nnn(C)c4C)cc3)[C@@H]3CCCNC3)c12]

Example 770 was prepared according to the procedure described for Example 614 by substituting 4-bromo-1-methyl-1H-1,2,3-triazole with 4-bromo-1,5-dimethyl-1H-1,2,3-triazole to afford the title compound as a pale brown solid. LC-MS: [M+H⁺]=459.2. ¹H NMR (CD₃OD, 400 MHZ): δ 8.50 (d, J=5.2 Hz, 1H), 7.78 (d, J=5.6 Hz, 1H), 7.37-7.27 (m, 3H), 7.22-7.1 (m, 3H), 5.13-5.06 (m, 1H), 4.00 (s, 3H), 3.96-3.93 (m, 1H), 3.84-3.79 (m, 1H), 3.41-3.37 (m, 1H), 3.01-2.97 (m, 1H), 2.76 (s, 3H), 2.33 (s, 3H), 1.96-1.95 (m, 2H), 1.86-1.83 (m, 1H), 1.55-1.51 (m, 1H).

### Synthesis of (R)—N-(6-chloro-8-methylisoquinolin-1-yl)-4-(1,5-dimethyl-1H-1,2,3-triazol-4-yl)-N-(piperidin-3-yl)benzamide (Example 771)

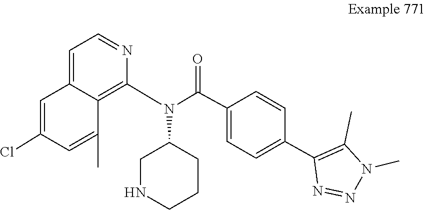 [SMILES: Cc1cc(Cl)cc2ccnc(N(C(=O)c3ccc(-c4nnn(C)c4C)cc3)[C@@H]3CCCNC3)c12]

Example 771 was prepared according to the procedure described for Example 816 by substituting 4-bromo-2-chloro-benzoyl chloride and 4-bromo-1-methyl-triazole with 4-bromo-benzoyl chloride and 4-bromo-1,5-dimethyl-1H-1,2,3-triazole, providing the title compound as a yellow solid. LC-MS: [M+H⁺]=475.3. ¹H NMR: (400 MHZ, CD₃OD): δ 8.56-8.48 (m, 1H), 7.78-7.70 (m, 2H), 7.37 (s, 1H), 7.34-7.27 (m, 2H), 7.18-7.11 (m, 2H), 5.17-5.06 (m, 1H), 4.03 (s, 3H), 3.99-3.91 (m, 1H), 3.87-3.78 (m, 1H), 3.45-3.36 (m, 1H), 3.08-2.94 (m, 1H), 2.83-2.72 (m, 3H), 2.35 (s, 3H), 2.04-1.81 (m, 3H), 1.62-1.47 (m, 1H).

### Synthesis of (R)—N-(6-chloro-8-methylisoquinolin-1-yl)-4-(5-methyl-1,3,4-oxadiazol-2-yl)-N-(piperidin-3-yl)benzamide (Example 772)

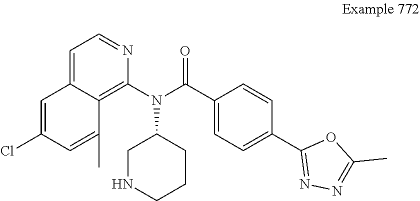 [SMILES: Cc1nnc(-c2ccc(C(=O)N(c3nccc4cc(Cl)cc(C)c34)[C@@H]3CCCNC3)cc2)o1]

Example 772 was prepared according to the procedure described for Example 816 by substituting 4-bromo-2-chloro-benzoyl chloride and 4-bromo-1-methyl-triazole with 4-bromo-benzoyl chloride and 2-bromo-5-methyl-1,3,4-oxadiazole, providing the title compound as a white solid. LC-MS: [M+H⁺]=462.2. ¹H NMR: (400 MHZ, CD₃OD): δ 8.56-8.45 (m, 1H), 7.75-7.56 (m, 4H), 7.37 (s, 1H), 7.22 (br d, J=8.4 Hz, 2H), 4.78-4.71 (m, 1H), 3.66 (br d, J=11.8 Hz, 1H), 3.37 (br d, J=11.0 Hz, 1H), 2.99 (br d, J=12.5 Hz, 1H), 2.80-2.72 (m, 3H), 2.53 (s, 3H), 2.51-2.40 (m, 1H), 1.93-1.63 (m, 3H), 1.37-1.28 (m, 1H).

### Synthesis of (R)—N-(6-chloro-8-methylisoquinolin-1-yl)-4-(3-methyl-1,2,4-oxadiazol-5-yl)-N-(piperidin-3-yl)benzamide (Example 773)

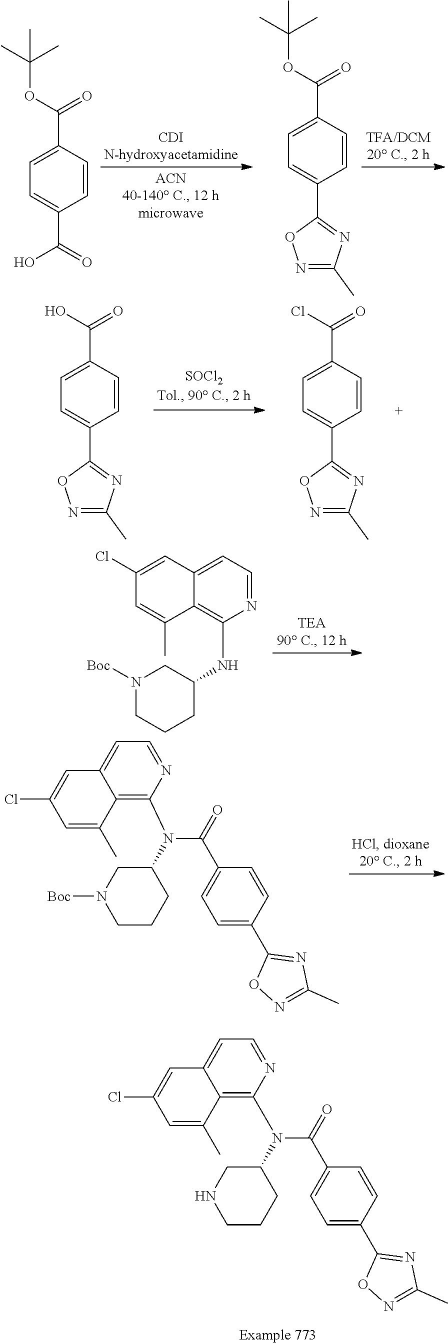

### Step 1:

To a solution of 4-tert-butoxycarbonylbenzoic acid (840 mg, 3.78 mmol) in MeCN (8.4 mL) was added CDI (735.46 mg, 4.54 mmol) at 20° C. The mixture was heated to 40° C. and stirred for 12 h. The reaction was then cooled down to 20° C., and N-hydroxyacetamidine (364.01 mg, 4.91 mmol) was added. The reaction mixture was heated at 140° C. under microwave irradiation for 45 min. The reaction mixture was concentrated, and the residue was purified by column chromatography (SiO₂, Petroleum ether/EtOAc=20/1 to 10/1) to give tert-butyl 4-(3-methyl-1,2,4-oxadiazol-5-yl) benzoate (830 mg, 84.3%) as a white solid.

### Step 2:

To a solution of tert-butyl 4-(3-methyl-1,2,4-oxadiazol-5-yl) benzoate (670 mg, 2.57 mmol) in DCM (3.4 mL) was added TFA (5.28 g, 46.33 mmol, 3.44 mL) at 20° C. The mixture was stirred at 20° C. for 2 h, after which it was concentrated to give 4-(3-methyl-1,2,4-oxadiazol-5-yl) benzoic acid (500 mg, 2.45 mmol, 95.13% yield) as a white solid. LC-MS: [M+H⁺]=205.1.

### Step 3:

A mixture of 4-(3-methyl-1,2,4-oxadiazol-5-yl) benzoic acid (250 mg, 1.22 mmol) and SOCl₂ (2.91 g, 24.49 mmol, 1.78 mL) in toluene (1.78 mL) was degassed and purged with N₂ 3 times at 20° C., and then the mixture was heated to 90° C. and stirred for 2 h under N₂. The reaction mixture was concentrated to give 4-(3-methyl-1,2,4-oxadiazol-5-yl) benzoyl chloride (270 mg, 99%) as a white solid.

### Steps 4-5:

Example 773 was prepared according to the procedure described for Example 444 by substituting 2-fluoro-4-(1-methyltriazol-4-yl)benzoyl chloride with 4-(3-methyl-1,2,4-oxadiazol-5-yl) benzoyl chloride, providing the title compound as a white solid. LC-MS: [M+H⁺]=462.4. ¹H NMR (400 MHZ, CD₃OD): δ 8.57-8.51 (m, 1H), 7.80-7.69 (m, 4H), 7.39 (s, 1H), 7.26-7.18 (m, 2H), 5.15-5.03 (m, 1H), 4.02-3.92 (m, 1H), 3.87-3.76 (m, 1H), 3.40 (br d, J=12.6 Hz, 1H), 3.08-2.94 (m, 1H), 2.83-2.72 (m, 3H), 2.36 (s, 3H), 2.04-1.82 (m, 3H), 1.63-1.48 (m, 1H).

### Synthesis of (R)—N-(6-fluoro-8-methylisoquinolin-1-yl)-4-(3-methyl-1,2,4-thiadiazol-5-yl)-N-(piperidin-3-yl)benzamide (Example 774)

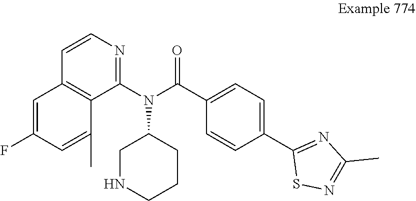 [SMILES: Cc1nsc(-c2ccc(C(=O)N(c3nccc4cc(F)cc(C)c34)[C@@H]3CCCNC3)cc2)n1]

Example 770 was prepared according to the procedure described for Example 614 by substituting 4-bromo-1-methyl-1H-1,2,3-triazole with 5-bromo-3-methyl-1,2,4-thiadiazole to afford the title compound as an off-white solid. LC-MS: [M+H⁺]=462.0. ¹H NMR (CD₃OD, 400 MHZ): δ 8.50 (d, J=5.6 Hz, 1H), 7.79 (d, J=5.6 Hz, 1H), 7.62-7.59 (m, 2H), 7.38-7.33 (m, 1H), 7.24-7.21 (m, 1H), 7.18-7.15 (m, 2H), 5.13-5.04 (m, 1H), 3.97-3.94 (m, 1H), 3.84-3.81 (m, 1H), 3.44-3.38 (m, 1H), 3.02-2.98 (m, 1H), 2.76 (s, 3H), 2.58 (s, 3H), 1.97-1.84 (m, 3H), 1.55-1.51 (m, 1H).

### Synthesis of (R)—N-(6-fluoro-8-methylisoquinolin-1-yl)-4-(3-methyl-1,2,4-oxadiazol-5-yl)-N-(piperidin-3-yl)benzamide (Example 775)

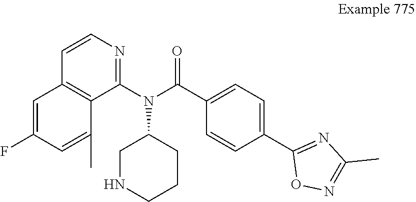 [SMILES: Cc1noc(-c2ccc(C(=O)N(c3nccc4cc(F)cc(C)c34)[C@@H]3CCCNC3)cc2)n1]

Example 775 was prepared according to the procedure described for Example 614 by substituting 4-bromo-1-methyl-1H-1,2,3-triazole with 5-bromo-3-methyl-1,2,4-oxadiazole to afford the title compound as an off-white solid. LC-MS: [M+H⁺]=446.3. ¹H NMR (CD₃OD, 400 MHZ): δ 8.50-8.45 (m, 1H), 7.78 (d, J=6.0 Hz, 1H), 7.75-7.69 (m, 2H), 7.37-7.32 (m, 1H), 7.24-7.18 (m, 3H), 5.11-5.02 (m, 1H), 3.97-3.94 (m, 1H), 3.84-3.78 (m, 1H), 3.41-3.37 (m, 1H), 3.02-2.98 (m, 1H), 2.76 (s, 3H), 2.35 (s, 3H), 1.98-1.83 (m, 3H), 1.57-1.52 (m, 1H).

### Synthesis of (R)-2-fluoro-N-(6-fluoro-8-methylisoquinolin-1-yl)-4-(5-methyl-1,3,4-oxadiazol-2-yl)-N-(piperidin-3-yl)benzamide (Example 776)

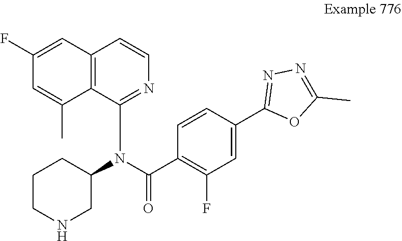 [SMILES: Cc1nnc(-c2ccc(C(=O)N(c3nccc4cc(F)cc(C)c34)[C@@H]3CCCNC3)c(F)c2)o1]

Example 776 was synthesized following the procedure for Example 833 by substituting 2-bromo-5-methyl-1,3,4-thiadiazole with 2-bromo-5-methyl-1,3,4-oxadiazole to afford the title compound as an off-white solid. LC-MS: [M+H⁺]=464.2. ¹H NMR (CD₃OD, 400 MHz): δ 8.53 (bs, 1H), 8.42-8.38 (m, 1H), 7.70-7.66 (m, 1H), 7.46-7.43 (m, 1H), 7.39-7.32 (m, 3H), 7.05-7.01 (m, 1H), 5.03-4.97 (m, 1H), 3.97-3.94 (m, 1H), 3.77-3.71 (m, 1H), 3.35-3.29 (m, 1H), 2.85 (s, 3H), 2.69-2.61 (m, 2H), 2.53 (s, 3H), 1.92-1.88 (m, 2H), 1.58-1.54 (m, 1H).

### Synthesis of (R)-2-methyl-4-(3-methyl-1,2,4-oxadiazol-5-yl)-N-(3-methylthieno[3,2-c]pyridin-4-yl)-N-(piperidin-3-yl)benzamide (Example 777)

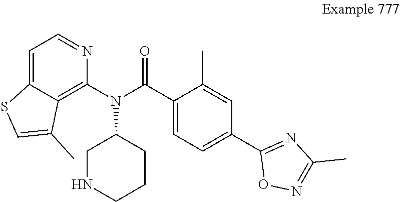 [SMILES: Cc1noc(-c2ccc(C(=O)N(c3nccc4scc(C)c34)[C@@H]3CCCNC3)c(C)c2)n1]

Example 777 was prepared according to the procedure described for Example 778 by substituting methyl 2-fluoro-4-(3-methyl-1,2,4-oxadiazol-5-yl) benzoate with tert-butyl 2-methyl-4-(3-methyl-1,2,4-oxadiazol-5-yl)benzoate, providing the title compound as a white solid. LC-MS: [M+H⁺]=448.2. ¹H NMR (400 MHZ, DMSO-d₆): δ 8.98-8.67 (m, 2H), 8.15 (br d, J=1.6 Hz, 1H), 7.97 (d, J=5.5 Hz, 1H), 7.79-7.63 (m, 2H), 7.46 (d, J=7.8 Hz, 1H), 7.05 (d, J=8.1 Hz, 1H), 4.94-4.57 (m, 1H), 3.24-3.20 (m, 1H), 2.69-2.66 (m, 2H), 2.65-2.61 (m, 3H), 2.45 (d, J=4.6 Hz, 4H), 2.33 (s, 3H), 1.85-1.68 (m, 3H), 1.11-0.79 (m, 1H).

### Synthesis of (R)-2-fluoro-4-(3-methyl-1,2,4-oxadiazol-5-yl)-N-(3-methylthieno[3,2-c]pyridin-4-yl)-N-(piperidin-3-yl)benzamide (Example 778)

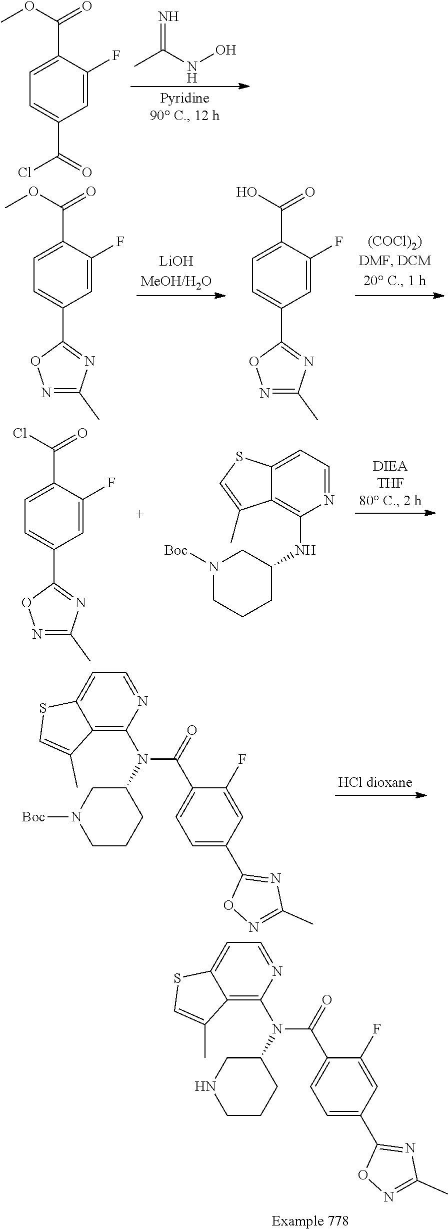

### Step 1:

To a solution of N-hydroxyacetamidine (205.22 mg, 2.77 mmol) in toluene (6 mL) were added pyridine (1.10 g, 13.85 mmol, 1.12 mL) and methyl 4-chlorocarbonyl-2-fluoro-benzoate (0.6 g, 2.77 mmol) at 20° C., and then the mixture was stirred at 90° C. for 12 h. The mixture was concentrated and purified by prep-TLC (EtOAc:petroleum ether=1:1) to give methyl 2-fluoro-4-(3-methyl-1, 2, 4-oxadiazol-5-yl) benzoate (150 mg, 22%) as a white solid.

### Step 2:

To a solution of methyl 2-fluoro-4-(3-methyl-1,2,4-oxadiazol-5-yl)benzoate (100 mg, 423.37 μmol) in MeOH (1 mL) and H₂O (1 mL) was added LiOH H₂O (53.30 mg, 1.27 mmol) at 20° C., and then the mixture was stirred at 20° C. for 2 h. The mixture was poured into water (20 mL) and acidified to pH 4 with 2 N HCl. The mixture was extracted with EtOAc (3×10 mL), and the combined organic phase was washed with saturated NaCl (2×10 mL), dried with anhydrous sodium sulfate, and concentrated to give 2-fluoro-4-(3-methyl-1,2,4-oxadiazol-5-yl)benzoic acid (94 mg, 99%) as a white solid. LC-MS: [M+H⁺]=223.1.

### Step 3:

To a solution of 2-fluoro-4-(3-methyl-1,2,4-oxadiazol-5-yl)benzoic acid (94 mg, 423.10 μmol) in DCM (2 mL) was added DMF (3.09 mg, 42.31 μmol, 3.26 μL) dropwise and (COCl)₂ (537.03 mg, 4.23 mmol, 370.37 μL) at 20° C., and then the mixture was stirred at 20° C. for 30 min. The mixture was concentrated to give 2-fluoro-4-(3-methyl-1, 2, 4-oxadiazol-5-yl)benzoylchloride (100 mg, 98%) as a brown solid.

### Steps 4-5:

Example 778 was prepared according to the procedure described for Example 444 by substituting tert-butyl (R)-3-((6-chloro-8-methylisoquinolin-1-yl)amino)piperidine-1-carboxylate and 2-fluoro-4-(1-methyltriazol-4-yl)benzoyl chloride with tert-butyl (R)-3-((3-methylthieno[3,2-c]pyridin-4-yl)amino)piperidine-1-carboxylate and 2-fluoro-4-(3-methyl-1,2,4-oxadiazol-5-yl) benzoyl chloride, providing the title compound as a white solid. LC-MS: [M+H⁺]=452.3 ¹H NMR (400 MHZ, DMSO-d₆): δ 9.19-9.02 (m, 1H), 9.02-8.87 (m, 1H), 8.80-8.10 (m, 2H), 8.06-7.84 (m, 1H), 7.74-7.50 (m, 3H), 7.27-7.13 (m, 1H), 5.00-4.64 (m, 1H), 3.84-3.74 (m, 1H), 3.22 (br d, J=12.4 Hz, 1H), 2.84-2.72 (m, 1H), 2.58-2.53 (m, 3H), 2.41-2.30 (m, 3H), 2.00-1.72 (m, 3H), 1.17-0.77 (m, 1H).

### Synthesis of (R)-2-fluoro-4-(3-methyl-1,2,4-thiadiazol-5-yl)-N-(3-methylthieno[3,2-c]pyridin-4-yl)-N-(piperidin-3-yl)benzamide (Example 779)

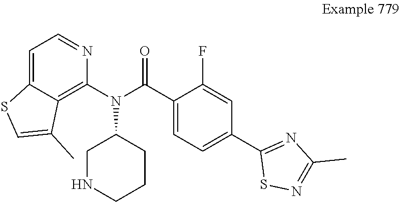 [SMILES: Cc1nsc(-c2ccc(C(=O)N(c3nccc4scc(C)c34)[C@@H]3CCCNC3)c(F)c2)n1]

Example 779 was prepared according to the procedure described for Example 782 by substituting 4-bromo-1,5-dimethyl-triazole with 5-bromo-3-methyl-1,2,4-thiadiazole, providing the title compound as a light brown solid. LC-MS: [M+H⁺]=468.2. ¹H NMR (400 MHz, DMSO-d₆): δ 9.13-8.83 (m, 2H), 8.47-8.31 (m, 1H), 8.14-8.01 (m, 1H), 7.90-7.59 (m, 2H), 7.54-7.26 (m, 1H), 7.20-6.98 (m, 1H), 5.05-4.62 (m, 1H), 3.83-3.72 (m, 1H), 3.30 (br d, J=11.0 Hz, 2H), 2.83-2.73 (m, 1H), 2.60-2.53 (m, 6H), 2.01-1.69 (m, 3H), 1.18-0.83 (m, 1H).

### Synthesis of 2-fluoro-N-(3-methylthieno[3,2-c]pyridin-4-yl)-N-[(3R)-3-piperidyl]-4-[5-(trideuteriomethyl)-1,3,4-thiadiazol-2-yl] benzamide (Example 780)

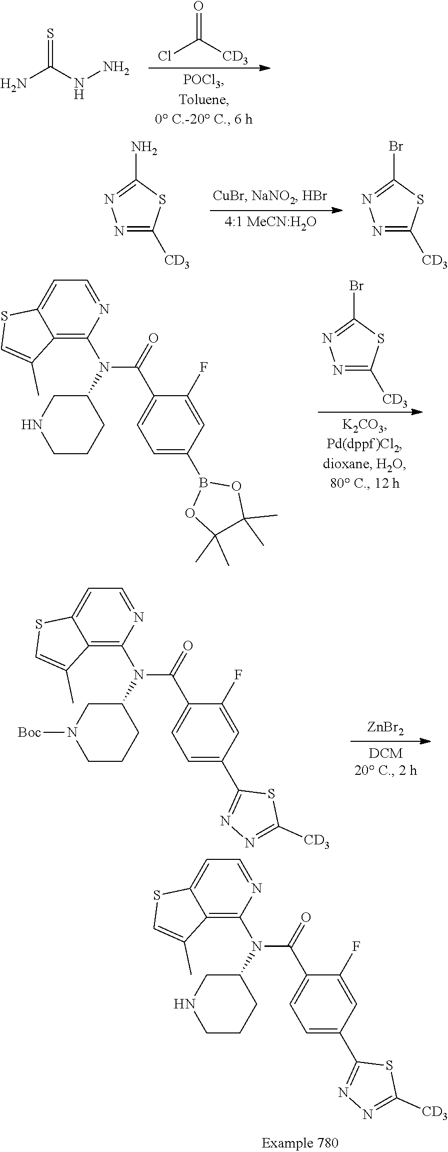

### Step 1:

To a solution of aminothiourea (0.5 g, 5.49 mmol) and 2,2,2-trideuterioacetyl chloride (491.95 mg, 6.03 mmol) in toluene (4 mL) was added POCl₃ (420.62 mg, 2.74 mmol, 255.69 μL) dropwise at 50° C., and then the mixture was stirred at 70° C. for 12 hours. The mixture was poured into water (20 mL) and extracted with EtOAc (3×10 mL). The combined organic phase were was washed with saturated NaCl (2×10 mL), dried with anhydrous sodium sulfate, and concentrated to give 5-(trideuteriomethyl)-1,3,4-thiadiazol-2-amine (0.6 g, 83%) as yellow solid.

### Step 2:

To a solution of 5-(trideuteriomethyl)-1,3,4-thiadiazol-2-amine (0.6 g, 4.57 mmol) in MeCN (6 mL) and H₂O (1 mL) was added NaNO₂ (378.33 mg, 5.48 mmol), and then HBr (4.62 g, 22.85 mmol, 3.10 mL, 40% purity) was added dropwise at 0° C. Then CuBr (786.59 mg, 5.48 mmol, 167.00 μL) was added at 0° C., and the mixture was stirred at 20° C. for 12 hours. The mixture was basified with solid Na₂CO₃ to pH 9, and the mixture was filtered through a pad of celite. The filter cake was washed with H₂O (3×10 mL) and extracted with EtOAc (3×20 mL). The combined organic phase was washed with saturated NaCl (3×20 mL), dried with anhydrous sodium sulfate, and concentrated to give 2-bromo-5-(trideuteriomethyl)-1,3,4-thiadiazole (0.5 g, 48%) as off white solid.

### Step 3:

To a solution of tert-butyl (3R)-3-[[2-fluoro-4-(4,4,5,5-tetramethyl-1,3,2-dioxaborolan-2-yl)benzoyl]-(3-methylthieno[3,2-c]pyridin-4-yl)amino]piperidine-1-carboxylate (100 mg, 167.92 μmol) and 2-bromo-5-(trideuteriomethyl)-1,3,4-thiadiazole (91.71 mg, 503.75 μmol) in dioxane (2.5 mL) and H₂O (0.5 mL) were added K₂CO₃ (69.62 mg, 503.75 μmol) and Pd(dppf)Cl₂ (12.29 mg, 16.79 μmol) at 20° C., and then the mixture was stirred at 100° C. for 1 hour under N₂. The mixture was poured into water (10 mL) and extracted with EtOAc (3×10 mL). The combined organic phase was washed with saturated NaCl (3×10 mL), dried with anhydrous sodium sulfate, and concentrated. The residue was purified by prep-TLC using 30% EtOAc in petroleum ether to give tert-butyl (3R)-3-[[2-fluoro-4-[5-(trideuteriomethyl)-1,3,4-thiadiazol-2-yl]benzoyl]-(3-methylthieno[3,2-c]pyridin-4-yl) amino] piperidine-1-carboxylate (60 mg, 50%) as white solid.

### Step 4:

To a solution of tert-butyl (3R)-3-[[2-fluoro-4-[5-(trideuteriomethyl)-1,3,4-thiadiazol-2-yl]benzoyl]-(3-methylthieno[3,2-c]pyridin-4-yl)amino]piperidine-1-carboxylate (60 mg, 105.13 μmol) in DCM (1 mL) was added ZnBr₂ (118.38 mg, 525.66 μmol, 26.31 μL) at 20° C., and the mixture was stirred at 20° C. for 6 hours. The mixture was concentrated and purified by reverse phase prep-HPLC to give 2-fluoro-N-(3-methylthieno[3, 2-c]pyridin-4-yl)-N-[(3R)-3-piperidyl]-4-[5-(trideuteriomethyl)-1, 3, 4-thiadiazol-2-yl] benzamide (17 mg, 33%) as white solid. ¹H NMR (400 MHZ, DMSO-d₆): δ=8.47-8.28 (m, 1H), 8.17-7.91 (m, 1H), 7.82-7.51 (m, 2H), 7.44-7.34 (m, 1H), 7.16-7.02 (m, 1H), 4.56-4.33 (m, 1H), 3.07-2.92 (m, 1H), 2.80 (br d, J=11.8 Hz, 1H), 2.64-2.54 (m, 3H), 2.30-1.89 (m, 3H), 1.82-1.20 (m, 3H), 0.98-0.52 (m, 1H).

### Synthesis of (R)-2-fluoro-4-(5-methyl-1,3,4-thiadiazol-2-yl)-N-(3-methylthieno[3,2-c]pyridin-4-yl)-N-(piperidin-3-yl)benzamide (Example 781)

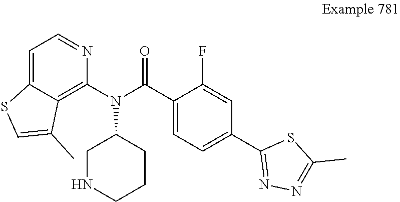 [SMILES: Cc1nnc(-c2ccc(C(=O)N(c3nccc4scc(C)c34)[C@@H]3CCCNC3)c(F)c2)s1]

Example 781 was prepared according to the procedure described for Example 127 by substituting tert-butyl (R)-3-((8-methylisoquinolin-1-yl)amino)piperidine-1-carboxylate with tert-butyl (R)-3-((3-methylthieno[3,2-c]pyridin-4-yl)amino)piperidine-1-carboxylate, providing the title compound as white solid. LC-MS: [M+H⁺]=468.2. ¹H NMR (400 MHZ, DMSO-d₆): δ=8.37-8.29 (m, 1H), 7.99-7.93 (m, 1H), 7.60-7.53 (m, 2H), 7.43-7.35 (m, 1H), 7.14-7.05 (m, 1H), 4.52-4.36 (m, 1H), 3.54-3.42 (m, 1H), 2.99 (br t, J=10.9 Hz, 1H), 2.84-2.76 (m, 2H), 2.72 (s, 3H), 2.64-2.60 (m, 1H), 2.52 (s, 3H), 2.32-2.16 (m, 2H), 2.05-1.90 (m, 1H), 1.68-1.36 (m, 2H), 0.93-0.77 (m, 1H).

### Synthesis of (R)-4-(1,5-dimethyl-1H-1,2,3-triazol-4-yl)-2-fluoro-N-(3-methylthieno[3,2-c]pyridin-4-yl)-N-(piperidin-3-yl)benzamide (Example 782)

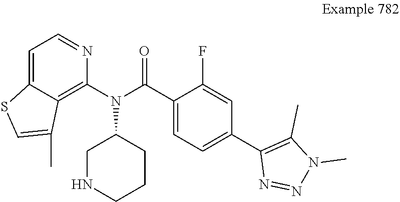 [SMILES: Cc1csc2ccnc(N(C(=O)c3ccc(-c4nnn(C)c4C)cc3F)[C@@H]3CCCNC3)c12]

Example 782 was prepared according to the procedure described for Example 699 by substituting 2-bromo-5-methyl-1,3,4-oxadiazole with 4-bromo-1,5-dimethyl-triazole, providing the title compound as a light brown solid. LC-MS: [M+H⁺]=465.2. ¹H NMR (400 MHz, DMSO-d₆): δ 9.36-8.69 (m, 2H), 8.48-8.31 (m, 1H), 8.22-7.97 (m, 1H), 7.73-7.57 (m, 1H), 7.33-7.23 (m, 1H), 7.20-7.11 (m, 1H), 7.08-6.94 (m, 1H), 5.04-4.28 (m, 1H), 3.91 (s, 3H), 3.77-3.73 (m, 1H), 3.50-3.21 (m, 2H), 2.84-2.67 (m, 1H), 2.57-2.52 (m, 3H), 2.32 (s, 3H), 1.97-1.60 (m, 3H), 1.18-0.83 (m, 1H).

### Synthesis of (R)-5-(5-methyl-1,3,4-thiadiazol-2-yl)-N-(8-methylisoquinolin-1-yl)-N-(piperidin-3-yl)picolinamide (Example 783)

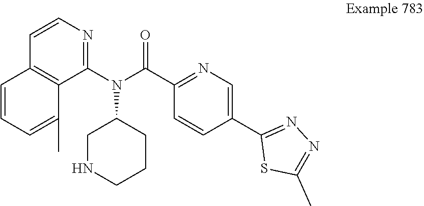 [SMILES: Cc1nnc(-c2ccc(C(=O)N(c3nccc4cccc(C)c34)[C@@H]3CCCNC3)nc2)s1]

Example 783 was prepared according to the procedure described for Example 622 by substituting 6-bromonicotinoyl chloride with 5-bromonicotinoyl chloride to afford the title compound as an off-white solid. LC-MS: [M+H⁺]=445.2. ¹H NMR (CD₃OD, 400 MHZ): δ 8.37-8.28 (m, 3H), 8.04-8.02 (m, 2H), 8.01-7.91 (m, 1H), 7.73-7.71 (m, 1H), 7.57-7.55 (m, 1H), 5.03-4.94 (m, 1H), 4.03-4.00 (m, 1H), 3.57-3.46 (m, 1H), 2.93-2.86 (m, 1H), 2.79 (s, 3H), 2.76 (s, 3H), 2.60-2.41 (m, 1H), 1.86-1.78 (m, 3H), 1.15-1.04 (m, 1H).

### Synthesis of (R)—N-(6-fluoro-8-methylisoquinolin-1-yl)-5-(5-methyl-1,3,4-thiadiazol-2-yl)-N-(piperidin-3-yl)picolinamide (Example 784)

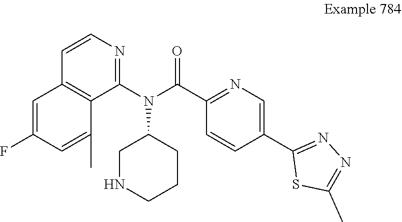 [SMILES: Cc1nnc(-c2ccc(C(=O)N(c3nccc4cc(F)cc(C)c34)[C@@H]3CCCNC3)nc2)s1]

Example 784 was prepared according to the procedure described for Example 783 by substituting (R)-8-methyl-N-(piperidin-3-yl)isoquinolin-1-amine with (R)-6-fluoro-8-methyl-N-(piperidin-3-yl)isoquinolin-1-amine to afford the title compound as an off-white solid. LC-MS: [M+H⁺]=463.3. ¹H NMR (400 MHZ, DMSO-d₆): δ 9.35-8.97 (m, 2H), 8.45-8.17 (m, 3H), 7.91-7.77 (m, 2H), 7.68-7.60 (m, 1H), 7.53-7.35 (m, 1H), 4.55-4.43 (m, 1H), 3.41-3.13 (m, 2H), 2.96-2.62 (m, 8H), 1.88-1.59 (m, 3H), 1.03-0.89 (m, 1H).

### Synthesis of (R)—N-(6-fluoro-8-methylisoquinolin-1-yl)-5-(1-methyl-1H-1,2,3-triazol-4-yl)-N-(piperidin-3-yl)picolinamide (Example 785)

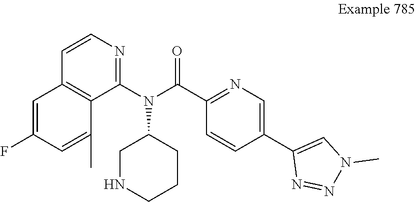 [SMILES: Cc1cc(F)cc2ccnc(N(C(=O)c3ccc(-c4cn(C)nn4)cn3)[C@@H]3CCCNC3)c12]

Example 785 was prepared according to the procedure described for Example 784 by substituting 2-bromo-1,3,4-thiadiazole with 4-bromo-1-methyl-1H-1,2,3-triazole to afford the title compound as an off-white solid. LC-MS: [M+H⁺]=446.2. ¹H NMR (CD₃OD, 400 MHz): δ 8.34-8.24 (m, 2H), 8.20-8.16 (m, 1H), 7.95-7.86 (m, 2H), 7.65 (d, J=8.4 Hz, 1H), 7.43-7.36 (m, 1H), 7.46-7.43 (m, 1H), 5.02-5.00 (m, 1H), 4.09 (s, 3H), 4.00-3.97 (m, 1H), 3.59-3.34 (m, 2H), 3.05-3.02 (m, 1H), 2.96-2.87 (m, 1H), 2.79 (s, 3H), 1.89-1.79 (m, 2H), 1.12-1.08 (m, 1H).

### Synthesis of (R)-4-((4-(benzylamino) pyrimidin-2-yl) amino)-2-fluoro-N-(8-methylisoquinolin-1-yl)-N-(piperidin-3-yl) benzamide (Example 786)

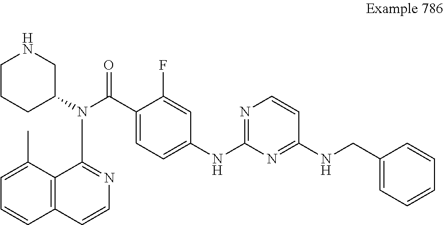 [SMILES: Cc1cccc2ccnc(N(C(=O)c3ccc(Nc4nccc(NCc5ccccc5)n4)cc3F)[C@@H]3CCCNC3)c12]

Example 786 was synthesized following the procedure for Example 210 by substituting 2-chloropyrimidine with N-benzyl-2-chloropyrimidin-4-amine to afford the title compound as a yellow solid. LC-MS: [M+H⁺]=562.30. ¹H NMR (CD₃OD, 400 MHZ): δ 8.37-8.34 (m, 1H), 8.17 (d, J=7.2 Hz, 1H), 7.67-7.57 (m, 2H), 7.45-7.36 (m, 5H), 7.27-7.24 (m, 2H), 7.09-7.02 (m, 1H), 6.76-6.70 (m, 2H), 6.26 (d, J=7.6 Hz, 1H), 5.07-5.01 (m, 1H), 4.51 (s, 2H), 3.98-3.94 (m, 1H), 3.82-3.76 (m, 1H), 3.40-3.37 (m, 1H), 3.01-2.98 (m, 1H), 2.80 (s, 3H), 2.14-1.89 (m, 3H), 1.54-1.50 (m, 1H).

### Synthesis of (R)-2-fluoro-N-(6-fluoroisoquinolin-1-yl)-4-((5-fluoropyrimidin-2-yl)amino)-N-(piperidin-3-yl)benzamide (Example 787)

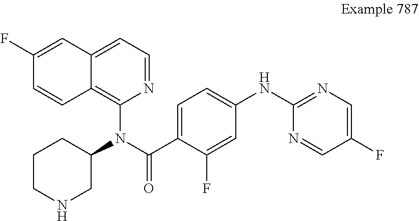 [SMILES: O=C(c1ccc(Nc2ncc(F)cn2)cc1F)N(c1nccc2cc(F)ccc12)[C@@H]1CCCNC1]

Example 787 was synthesized following the procedure for Example 409 by substituting 1-chloro-8-methyl-isoquinoline with 1-chloro-6-fluoroisoquinoline to afford the title compound as a pale-yellow solid. LC-MS: [M+H⁺]=479.25. ¹H NMR (CD₃OD, 400 MHz): δ 8.43 (d, J=5.6 Hz, 1H), 8.36 (s, 2H), 8.19-8.14 (m, 1H), 7.74-7.73 (m, 1H), 7.60-7.48 (m, 3H), 6.92-6.85 (m, 2H), 5.25-5.23 (m, 1H), 3.89-3.86 (m, 1H), 3.75-3.66 (m, 1H), 3.39-3.34 (m, 1H), 2.85-2.83 (m, 1H), 1.98-1.88 (m, 3H), 1.28-1.25 (m, 1H).

### Synthesis of (R)—N-(6-chloroisoquinolin-1-yl)-2-fluoro-4-(5-methyl-1,3,4-oxadiazol-2-yl)-N-(piperidin-3-yl)benzamide (Example 788)

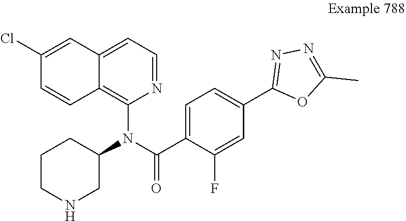 [SMILES: Cc1nnc(-c2ccc(C(=O)N(c3nccc4cc(Cl)ccc34)[C@@H]3CCCNC3)c(F)c2)o1]

Example 788 was prepared according to the procedure described for Example 127 by substituting tert-butyl (R)-3-((8-methylisoquinolin-1-yl)amino)piperidine-1-carboxylate with tert-butyl (R)-3-((6-chloroisoquinolin-1-yl)amino)piperidine-1-carboxylate, provided the title compound hydrochloride salt as an off-white solid. LC-MS: [M+H⁺]=466.2. ¹H NMR (CD₃OD, 400 MHZ): δ 8.54-8.40 (m, 1H), 8.17 (d, J=9.2 Hz, 1H), 7.94-7.90 (m, 1H), 7.74-7.67 (m, 2H), 7.47-7.41 (m, 2H), 7.31-7.21 (m, 1H), 5.24-5.17 (m, 1H), 3.93-3.91 (m, 1H), 3.66-3.60 (m, 1H), 3.21-3.12 (m, 1H), 2.86-2.81 (m, 1H), 2.51 (s, 3H), 2.03-1.97 (m, 1H), 1.90-1.83 (m, 2H), 1.28-1.18 (m, 1H).

### Synthesis of (R)-4-(5-cyclopropyl-1,3,4-oxadiazol-2-yl)-N-(8-methylisoquinolin-1-yl)-N-(piperidin-3-yl)benzamide (Example 789)

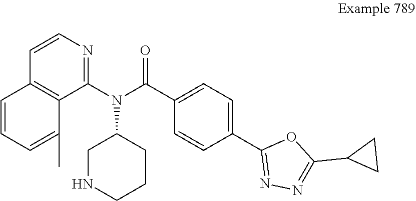 [SMILES: Cc1cccc2ccnc(N(C(=O)c3ccc(-c4nnc(C5CC5)o4)cc3)[C@@H]3CCCNC3)c12]

Example 789 was prepared according to the procedure described for Example 784 by substituting 2-bromo-1,3,4-thiadiazole with 2-bromo-5-cyclopropyl-1,3,4-oxadiazole and (R)—N-(6-fluoro-8-methylisoquinolin-1-yl)-N-(piperidin-3-yl)-5-(4,4,5,5-tetramethyl-1,3,2-dioxaborolan-2-yl)picolinamide with (R)—N-(8-methylisoquinolin-1-yl)-N-(piperidin-3-yl)-4-(4,4,5,5-tetramethyl-1,3,2-dioxaborolan-2-yl)benzamide to afford the title compound as an off-white solid. LC-MS: [M+H⁺]=454.2. ¹H NMR (CD₃OD, 400 MHZ): δ 8.50-8.45 (m, 1H), 7.80-7.75 (m, 1H), 7.67-7.63 (m, 1H), 7.58-7.55 (m, 2H), 7.48-7.44 (m, 1H), 7.40-7.37 (m, 1H), 7.19-7.17 (m, 2H), 5.09-5.01 (m, 1H), 3.94-3.91 (m, 1H), 3.79-3.74 (m, 1H), 3.47-3.45 (m, 1H), 2.94-2.90 (m, 1H), 2.80 (s, 3H), 2.67-2.65 (m, 1H), 2.21-2.17 (m, 1H), 1.93-1.90 (m, 2H), 1.51-1.47 (m, 1H), 1.19-1.17 (m, 2H), 1.11-1.10 (m, 2H).

### Synthesis of (R)-4-(5-cyclopropyl-1,3,4-thiadiazol-2-yl)-N-(8-methylisoquinolin-1-yl)-N-(piperidin-3-yl) benzamide (Example 790)

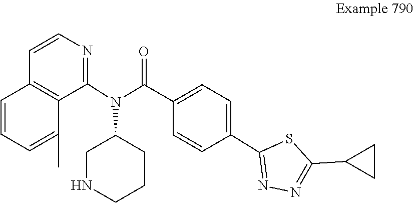 [SMILES: Cc1cccc2ccnc(N(C(=O)c3ccc(-c4nnc(C5CC5)s4)cc3)[C@@H]3CCCNC3)c12]

Example 790 was prepared according to the procedure described for Example 789 by substituting 2-bromo-5-cyclopropyl-1,3,4-oxadiazole with 2-bromo-5-cyclopropyl-1,3,4-thiadiazole to afford the title compound as an off-white solid. LC-MS: [M+H⁺]=470.2. ¹H NMR (CD₃OD, 400 MHZ): δ 8.51-8.49 (m, 1H), 7.82-7.80 (m, 1H), 7.69-7.66 (m, 1H), 7.52-7.39 (m, 4H), 7.16-7.14 (m, 2H), 5.11-5.05 (m, 1H), 3.97-3.94 (m, 1H), 3.83-3.77 (m, 1H), 3.40-3.37 (m, 1H), 3.02-2.98 (m, 1H), 2.75 (s, 3H), 2.46-2.42 (m, 1H), 1.94-1.83 (m, 3H), 1.51-1.48 (m, 1H), 1.28-1.25 (m, 2H), 1.08-1.07 (m, 2H).

### Synthesis of (R)-4-(5-ethyl-1,3,4-thiadiazol-2-yl)-N-(8-methylisoquinolin-1-yl)-N-(piperidin-3-yl) benzamide (Example 791)

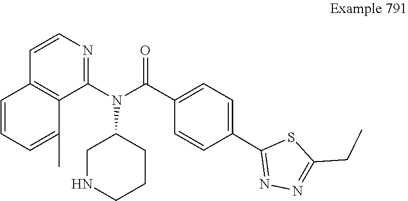 [SMILES: CCc1nnc(-c2ccc(C(=O)N(c3nccc4cccc(C)c34)[C@@H]3CCCNC3)cc2)s1]

Example 791 was prepared according to the procedure described for Example 789 by substituting 2-bromo-5-cyclopropyl-1,3,4-oxadiazole with 2-bromo-5-ethyl-1,3,4-thiadiazole to afford the title compound as an off-white solid. LC-MS: [M+H⁺]=458.6. ¹H NMR (DMSO-d₆, 400 MHZ): δ 8.51 (d, J=5.6 Hz, 1H), 7.81 (d, J=5.6 Hz, 1H), 7.69-7.65 (m, 1H), 7.56-7.53 (m, 2H), 7.47 (t, J=7.6 Hz, 1H), 7.39 (t, J=6.8 Hz, 1H), 7.17-7.14 (m, 2H), 5.13-5.05 (m, 1H), 3.98-3.95 (m, 1H), 3.84-3.78 (m, 1H), 3.40-3.39 (m, 1H), 3.10 (q, J=7.6 Hz, 2H), 2.99-2.98 (m, 1H), 2.75 (s, 3H), 1.95-1.87 (m, 3H), 1.52-1.48 (m, 1H), 1.38 (t, J=7.6 Hz, 3H).

### Synthesis of (R)-4-(5-methyl-1,3,4-thiadiazol-2-yl)-N-(8-methylisoquinolin-1-yl)-N-(piperidin-3-yl) benzamide (Example 792)

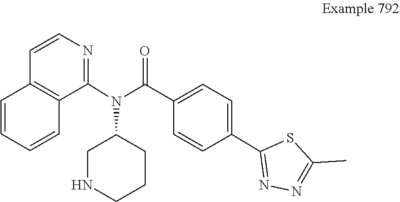 [SMILES: Cc1nnc(-c2ccc(C(=O)N(c3nccc4ccccc34)[C@@H]3CCCNC3)cc2)s1]

Example 792 was prepared according to the procedure described for Example 789 by substituting 2-bromo-5-cyclopropyl-1,3,4-oxadiazole with 2-bromo-5-methyl-1,3,4-thiadiazole to afford the title compound as an off-white solid. LC-MS: [M+H⁺]=444.2. ¹H NMR (DMSO-d₆, 400 MHZ): δ 8.52 (d, J=5.6 Hz, 1H), 7.80 (d, J=5.6 Hz, 1H), 7.72-7.62 (m, 1H), 7.54 (bs, 1H), 7.52 (bs, 1H), 7.47 (t, J=7.2 Hz, 1H), 7.39-7.37 (m, 1H), 7.17-7.10 (m, 2H), 5.06-4.95 (m, 1H), 3.93-3.90 (m, 1H), 3.76-3.70 (m, 1H), 3.25-3.22 (m, 1H), 2.94-2.89 (m, 1H), 2.75 (s, 3H), 2.73 (s, 3H), 1.93-1.80 (m, 3H), 1.52-1.39 (m, 1H).

### Synthesis of N-(6-chloro-8-methyl-1-isoquinolyl)-2-fluoro-N-[(3R)-3-piperidyl]-4-(pyrimidin-2-ylamino)benzamide (Example 793)

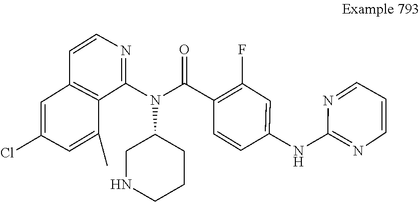 [SMILES: Cc1cc(Cl)cc2ccnc(N(C(=O)c3ccc(Nc4ncccn4)cc3F)[C@@H]3CCCNC3)c12]

Example 793 was synthesized following the procedure for Example 210 by substituting 1-chloro-8-methylisoquinoline with 1,6-dichloro-8-methylisoquinoline to afford the title compound as a white solid. LC-MS: [M+H⁺]=491.3. ¹H NMR (400 MHZ, CD₃OD): δ=8.63 (d, J=5.1 Hz, 2H), 8.50-8.37 (m, 1H), 7.78-7.64 (m, 2H), 7.48-7.30 (m, 2H), 7.12 (t, J=5.3 Hz, 1H), 6.93 (dd, J=1.8, 8.5 Hz, 1H), 6.85-6.74 (m, 1H), 5.16-4.99 (m, 1H), 4.01-3.91 (m, 1H), 3.86-3.77 (m, 1H), 3.49-3.37 (m, 1H), 3.09-2.93 (m, 1H), 2.86-2.75 (m, 3H), 2.22-1.84 (m, 3H), 1.60 (dq, J=5.8, 11.8 Hz, 1H).

### Synthesis of (R)—N-(6-chloroisoquinolin-1-yl)-2-fluoro-N-(piperidin-3-yl)-4-(pyrimidin-2-ylamino)benzamide (Example 794)

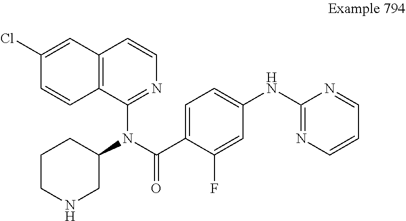 [SMILES: O=C(c1ccc(Nc2ncccn2)cc1F)N(c1nccc2cc(Cl)ccc12)[C@@H]1CCCNC1]

Synthesis of Example 794 is described in Example 738. Example 738 was isolated as an off-white solid. LC-MS: [M+H⁺]=536.2. ¹H NMR (CD₃OD, 400 MHZ): δ ppm 8.62-8.61 (m, 3H), 8.47 (bs, 2H), 8.34-8.33 (m, 1H), 8.12-8.11 (m, 1H), 7.95 (bs, 1H), 7.84 (bs, 1H), 7.58 (bs, 1H), 7.14-7.04 (m, 3H), 6.91-6.90 (m, 1H), 5.12-5.06 (m, 1H), 3.96-3.94 (m, 1H), 3.67-3.63 (m, 1H), 3.96-3.93 (m, 1H), 2.93-2.90 (m, 1H), 2.03-1.93 (m, 3H), 1.48-1.36 (m, 1H).

### Synthesis of (R)—N-(3-chlorothieno[3,2-c]pyridin-4-yl)-2-fluoro-4-(1-methyl-1H-1,2,3-triazol-4-yl)-N-(piperidin-3-yl)benzamide (Example 795)

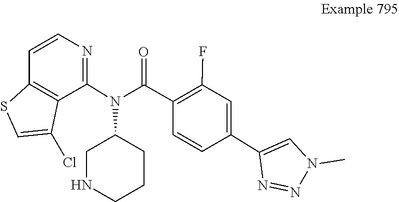 [SMILES: Cn1cc(-c2ccc(C(=O)N(c3nccc4scc(Cl)c34)[C@@H]3CCCNC3)c(F)c2)nn1]

Example 795 was prepared according to the procedure described for Example 444 by substituting tert-butyl (3R)-3-[(6-chloro-8-methyl-1-isoquinolyl)amino]piperidine-1-carboxylate with tert-butyl (3R)-3-[(3-chlorothieno [3, 2-c] pyridin-4-yl) amino] piperidine-1-carboxylate, providing the title compound as a white solid. LC-MS: [M+H⁺]=471.2. ¹H NMR (400 MHZ, CD₃OD): δ 8.45 (d, J=5.6 Hz, 1H), 8.23 (s, 1H), 7.97 (d, J=5.5 Hz, 1H), 7.82-7.74 (m, 1H), 7.35-7.26 (m, 2H), 7.26-7.18 (m, 1H), 5.12 (tt, J=3.9, 11.8 Hz, 1H), 4.09 (s, 3H), 3.98-3.87 (m, 1H), 3.68 (t, J=11.8 Hz, 1H), 3.41-3.33 (m, 1H), 2.91 (dt, J=4.5, 12.3 Hz, 1H), 2.04-1.80 (m, 3H), 1.27 (dq, J=5.2, 11.9 Hz, 1H).

### Synthesis of (R)-4-(3-methylisoxazol-5-yl)-N-(3-methylthieno[3,2-c]pyridin-4-yl)-N-(piperidin-3-yl)piperidine-1-carboxamide (Example 796)

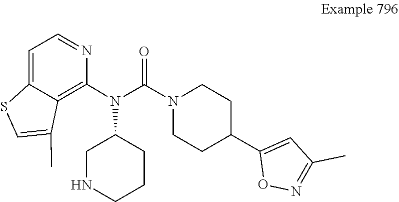 [SMILES: Cc1cc(C2CCN(C(=O)N(c3nccc4scc(C)c34)[C@@H]3CCCNC3)CC2)on1]

Example 796 was prepared according to the procedure described for Example 243 by substituting tert-butyl (3R)-3-[(8-methyl-1-isoquinolyl)-[4-(3-methylisoxazol-5-yl)piperidine-1-carbonyl]amino]piperidine-1-carboxylate with tert-butyl (R)-3-((chlorocarbonyl)(3-methylthieno[3,2-c]pyridin-4-yl)amino)piperidine-1-carboxylate, provided the title compound as a white solid. LC-MS: [M+H⁺]=440.3. ¹H NMR (400 MHZ, CD₃OD): δ 8.41 (d, J=5.9 Hz, 1H), 8.24 (d, J=5.9 Hz, 1H), 7.71 (br s, 1H), 5.86 (s, 1H), 4.74-4.34 (m, 1H), 3.85 (br s, 1H), 3.74-3.47 (m, 2H), 3.40-3.31 (m, 1H), 2.94 (br t, J=12.1 Hz, 1H), 2.85-2.67 (m, 3H), 2.57 (br s, 3H), 2.19 (s, 3H), 2.09-1.95 (m, 1H), 1.93-1.68 (m, 3H), 1.60 (br d, J=3.1 Hz, 1H), 1.13 (br d, J=11.0 Hz, 1H), 0.88-0.57 (m, 1H).

### Synthesis of (R)-5-(3-methylisoxazol-5-yl)-N-(3-methylthieno[3,2-c]pyridin-4-yl)-N-(piperidin-3-yl)picolinamide (Example 797)

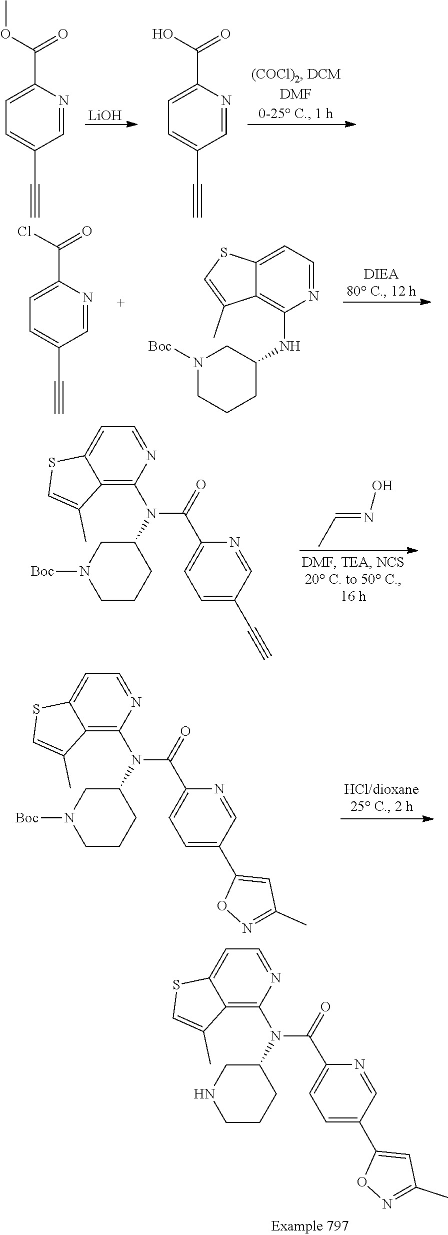

### Step 1:

To a solution of methyl 5-ethynylpyridine-2-carboxylate (220 mg, 1.37 mmol) in THF (1.65 mL), H₂O (0.27 mL), and MeOH (0.27 mL) was added LiOH (65.39 mg, 2.73 mmol). The mixture was stirred at 25° C. for 1 h. The reaction mixture was adjusted to pH 4 with 1 N HCl and extracted with EtOAc (3×3 mL). The combined organic phase was washed with brine (3 mL), dried over Na₂SO₄, filtered, and concentrated to give 5-ethynylpyridine-2-carboxylic acid (180 mg, 89%) as a white solid.

### Step 2:

To a solution of 5-ethynylpyridine-2-carboxylic acid (180 mg, 1.22 mmol) in DCM (2 mL) was added oxalyl dichloride (310.5 mg, 2.45 mmol, 214.18 μL) and DMF (8.94 mg, 122.34 μmol) at 0° C. The reaction mixture was stirred at 25° C. for 1 h, after which it was concentrated to give 5-ethynylpyridine-2-carbonyl chloride (180 mg, 88%) as a yellow solid.

### Step 3:

To a solution of tert-butyl (3R)-3-[(3-methylthieno[3,2-c]pyridin-4-yl)amino]piperidine-1-carboxylate (200 mg, 575.58 μmol) in toluene (4 mL) were added DIEA (371.95 mg, 2.88 mmol) and 5-ethynylpyridine-2-carbonyl chloride (95.30 mg, 575.58 μmol). The mixture was stirred at 80° C. for 12 h. The reaction mixture was quenched with MeOH (1 mL) and concentrated. The residue was purified by prep-TLC (petroleum ether:EtOAc=1:1) to give tert-butyl (R)-3-(5-ethynyl-N-(3-methylthieno[3,2-c]pyridin-4-yl)picolinamido) piperidine-1-carboxylate (150 mg, 54%) as a yellowish oil.

### Step 4:

To a solution of (1E)-acetaldehyde oxime hydrochloride (30.07 mg, 314.74 μmol) in DMF (4 mL) was added NCS (46.23 mg, 346.21 μmol) at 0° C. The mixture was stirred at 25° C. for 12 h. Then tert-butyl (R)-3-(5-ethynyl-N-(3-methylthieno[3,2-c]pyridin-4-yl)picolinamido) piperidine-1-carboxylate (150.0 mg, 314.74 μmol) was added, and the reaction mixture was stirred at 25° C. for 1 h. TEA (63.70 mg, 629.47 μmol) was added to the reaction mixture, and the mixture was stirred at 50° C. for 3 h under N₂. The reaction mixture was diluted with water (10 mL), the suspension was filtered, and the filter cake was dried to give a residue, which was purified by prep-TLC (petroleum ether:EtOAc=1:1) to give tert-butyl (R)-3-(5-(3-methylisoxazol-5-yl)-N-(3-methylthieno[3,2-c]pyridin-4-yl)picolinamido) piperidine-1-carboxylate (80 mg, 47%) as a yellow solid.

### Step 5:

A solution of tert-butyl (R)-3-(5-(3-methylisoxazol-5-yl)-N-(3-methylthieno[3,2-c]pyridin-4-yl)picolinamido) piperidine-1-carboxylate (80 mg, 149.91 μmol) in 4 M HCl/dioxane (1 mL) was stirred at 25° C. for 2 h. The mixture was concentrated to give the title compound (43 mg, 71%) as a white solid. LC-MS: [M+H⁺]=434.2. ¹H NMR (400 MHZ, DMSO-d₆): δ 0.31-0.59 (m, 2H) 0.90-1.18 (m, 3H) 1.48 (s, 3H) 1.65-1.81 (m, 3H) 2.02-2.25 (m, 1H) 2.55 (br d, J=12.01 Hz, 1H) 2.67-2.87 (m, 1H) 3.17 (br d, J=11.38 Hz, 1H) 4.26 (br t, J=11.07 Hz, 1H) 5.93 (s, 1H) 6.68-6.86 (m, 1H) 7.09-7.26 (m, 1H) 7.29-7.47 (m, 2H) 7.49-7.70 (m, 2H).

### Synthesis of (R)-4-(5-methyl-1,3,4-thiadiazol-2-yl)-N-(3-methylthieno[3,2-c]pyridin-4-yl)-N-(piperidin-3-yl)benzamide (Example 798)

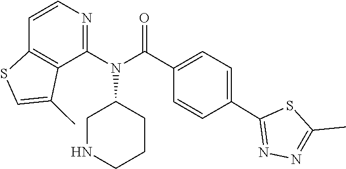 [SMILES: Cc1nnc(-c2ccc(C(=O)N(c3nccc4scc(C)c34)[C@@H]3CCCNC3)cc2)s1]

Example 798 was prepared according to the procedure described for Example 792 by substituting (R)-8-methyl-N-(piperidin-3-yl)isoquinolin-1-amine with (R)-3-methyl-N-(piperidin-3-yl)thieno[3,2-c]pyridin-4-amine to afford the title compound as an off-white solid. LC-MS: [M+H⁺]=450.1. ¹H NMR (DMSO-d₆, 400 MHZ): δ 0.99-1.18 (m, 1H) 1.51-1.76 (m, 2H) 2.45 (br d, J=7.13 Hz, 1H) 2.47-2.55 (m, 3H) 2.75 (s, 3H) 2.80-2.88 (m, 1H) 2.96 (br d, J=12.38 Hz, 1H) 3.23-3.28 (m, 1H) 3.61 (br d, J=12.13 Hz, 1H) 4.61-4.76 (m, 1H) 7.25-7.36 (m, 3H) 7.62 (d, J=8.50 Hz, 2H) 7.81-7.93 (m, 1H) 8.35-8.44 (m, 1H).

### Synthesis of (R)-4-(1-methyl-1H-1,2,3-triazol-4-yl)-N-(3-methylthieno[3,2-c]pyridin-4-yl)-N-(piperidin-3-yl)benzamide (Example 799)

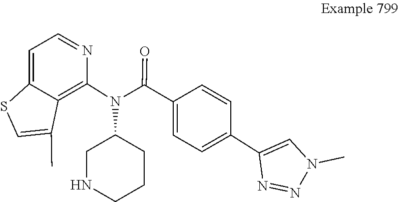 [SMILES: Cc1csc2ccnc(N(C(=O)c3ccc(-c4cn(C)nn4)cc3)[C@@H]3CCCNC3)c12]

Example 799 was prepared according to the procedure described for Example 842 by substituting tert-butyl (R)-3-((3-(prop-1-en-2-yl)thieno[3,2-c]pyridin-4-yl)amino)piperidine-1-carboxylate and 2-fluoro-4-(1-methyl-1H-1,2,3-triazol-4-yl)benzoyl chloride with tert-butyl (3R)-3-[(3-methylthieno [3, 2-c] pyridin-4-yl) amino] piperidine-1-carboxylate and 4-(1-methyl-1H-1,2,3-triazol-4-yl)benzoyl chloride, providing the title compound as a white solid. LC-MS: [M+H⁺]=433.2. ¹H NMR (400 MHZ, CD₃OD): δ 8.46 (d, J=5.6 Hz, 1H), 8.30-8.20 (m, 1H), 8.01 (d, J=5.5 Hz, 1H), 7.52 (br d, J=8.1 Hz, 2H), 7.43-7.34 (m, 1H), 7.26 (br d, J=8.3 Hz, 2H), 5.21-5.06 (m, 1H), 4.11 (s, 3H), 3.92 (br d, J=9.5 Hz, 1H), 3.83-3.70 (m, 1H), 3.37 (br d, J=12.5 Hz, 1H), 3.02-2.84 (m, 1H), 2.61-2.47 (m, 3H), 1.99-1.85 (m, 2H), 1.78 (br d, J=12.1 Hz, 1H), 1.42-1.21 (m, 1H).

### Synthesis of (R)—N-(8-chloroisoquinolin-1-yl)-4-(1-methyl-1H-1,2,3-triazol-4-yl)-N-(piperidin-3-yl)benzamide (Example 800)

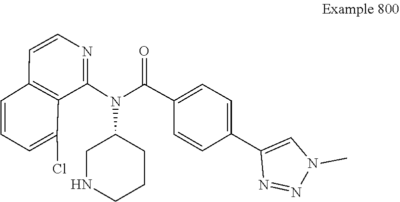 [SMILES: Cn1cc(-c2ccc(C(=O)N(c3nccc4cccc(Cl)c34)[C@@H]3CCCNC3)cc2)nn1]

Example 800 was prepared according to the procedure described for Example 111 by substituting (R)-8-methyl-N-(piperidin-3-yl)isoquinolin-1-amine with (R)-8-chloro-N-(piperidin-3-yl)isoquinolin-1-amine to afford the title compound as a yellow solid. LC-MS: [M+H⁺]=447.2. ¹H NMR (CD₃OD, 400 MHZ): δ 8.59-8.56 (m, 1H), 8.13 (s, 1H), 7.87-7.83 (m, 1H), 7.79-7.72 (m, 1H), 7.65-7.63 (m, 1H), 7.52-7.48 (m, 1H), 7.41-7.39 (m, 2H), 7.10-7.09 (m, 2H), 5.14-5.08 (m, 1H), 4.06 (s, 3H), 3.96-3.92 (m, 1H), 3.79-3.73 (m, 1H), 3.40-3.37 (m, 1H), 3.13-2.96 (m, 1H), 2.64-2.63 (m, 1H), 1.98-1.93 (m, 2H), 1.45-1.42 (m, 1H).

### Synthesis of (R)—N-(6-chloro-8-methylisoquinolin-1-yl)-5-(5-methyl-1,3,4-thiadiazol-2-yl)-N-(piperidin-3-yl)picolinamide (Example 801)

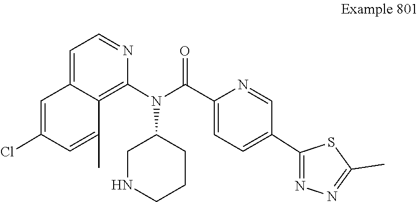 [SMILES: Cc1nnc(-c2ccc(C(=O)N(c3nccc4cc(Cl)cc(C)c34)[C@@H]3CCCNC3)nc2)s1]

Example 801 was prepared according to the procedure described for Example 816 by substituting 4-bromo-2-chloro-benzoyl chloride and 4-bromo-1-methyl-triazole with 5-bromopyridine-2-carbonyl chloride and 2-bromo-5-methyl-1, 3, 4-thiadiazole, provided the title compound as a white solid. LC-MS: [M+H⁺]=479.2. ¹H NMR (400 MHZ, CD₃OD): δ ppm 8.37 (d, J=1.5 Hz, 1H), 8.29-8.18 (m, 2H), 7.94-7.84 (m, 1H), 7.81 (d, J=1.9 Hz, 1H), 7.75-7.65 (m, 1H), 7.51-7.42 (m, 1H), 4.70-4.37 (m, 1H), 3.69 (br d, J=12.0 Hz, 1H), 3.12-2.98 (m, 1H), 2.92-2.81 (m, 4H), 2.76 (s, 3H), 2.44-2.30 (m, 1H), 1.88-1.49 (m, 3H), 0.99-0.60 (m, 1H).

### Synthesis of (R)—N-(6-chloro-8-methylisoquinolin-1-yl)-4-(1-methyl-1H-1,2,3-triazol-4-yl)-N-(piperidin-3-yl)benzamide (Example 802)

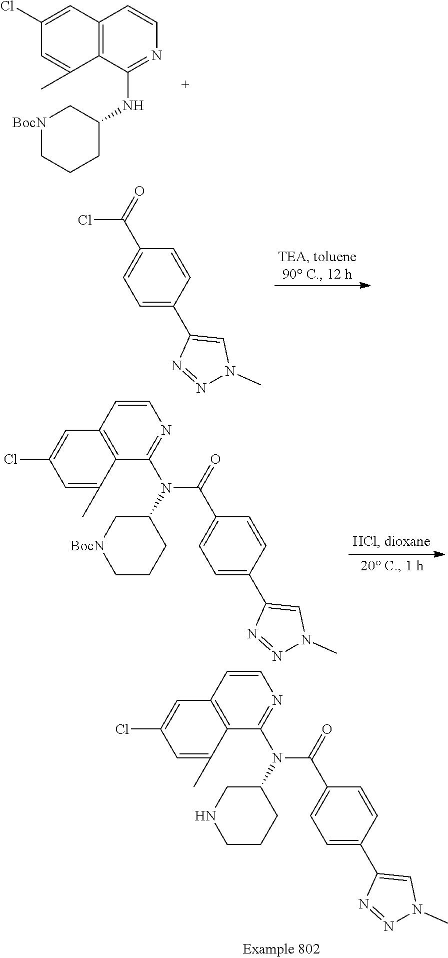

### Step 1:

To a solution of crude 4-(1-methyl-1H-1,2,3-triazol-4-yl)benzoyl chloride (5.31 g, 23.96 mmol) which was prepared according to the procedure for Example 111, in toluene (60 mL) at 0° C. under N₂ atmosphere was added TEA (5.55 mL, 39.91 mmol) and followed by portion-wise addition of tert-butyl (R)-3-((6-chloro-8-methylisoquinolin-1-yl)amino)piperidine-1-carboxylate (3.00 g, 7.98 mmol), which was prepared according to procedure for Example 444. The reaction was heated to 90° C. and stirred for 12 h. The reaction was cooled and concentrated under reduced pressure. The residue was purified by flash column chromatography (SiO₂, eluting with petroleum ether/EtOAc=3/1 to 2/1) to provide tert-butyl (R)-3-(N-(6-chloro-8-methylisoquinolin-1-yl)-4-(1-methyl-1H-1,2,3-triazol-4-yl)benzamido)piperidine-1-carboxylate (2.50, 56%) as an off-white solid.

### Step 2:

To a solution of tert-butyl (R)-3-(N-(6-chloro-8-methylisoquinolin-1-yl)-4-(1-methyl-1H-1,2,3-triazol-4-yl)benzamido)piperidine-1-carboxylate (4.00 g, 7.13 mmol) in 1,4-dioxane (40 mL) at 20° C. was added a solution of 4M HCl in 1,4-dioxane (8.91 mL). The reaction mixture was stirred for 1 h and then it was concentrated under reduced pressure. The residue was purified by reverse-phase Prep-HPLC (column: Welch Xtimate C18 250*100 mm*10 μm; mobile phase: [H₂O (10 mM NH₄HCO₃)-MeCN]; gradient: 15%-45% B over 20.0 min) and the pure fraction was dried by lyophilization to give the title compound (1.20 g, 37%) as a white solid. LC-MS: [M+H⁺]=461.3. ¹H NMR (400 MHz, CD₃OD): δ ppm 8.53-8.43 (m, 1H), 8.12 (s, 1H), 7.72-7.62 (m, 2H), 7.42 (d, J=8.4 Hz, 2H), 7.35 (s, 1H), 7.14-7.02 (m, 2H), 4.79-4.58 (m, 1H), 4.07 (s, 3H), 3.64 (br d, J=12.0 Hz, 1H), 3.39-3.34 (m, 1H), 2.98 (br d, J=12.5 Hz, 1H), 2.79-2.68 (m, 3H), 2.57-2.43 (m, 1H), 1.98-1.55 (m, 3H), 1.43-1.23 (m, 1H).

### Synthesis of (R)—N-(6-fluoroisoquinolin-1-yl)-5-(1-methyl-1H-1,2,3-triazol-4-yl)-N-(piperidin-3-yl)picolinamide (Example 803)

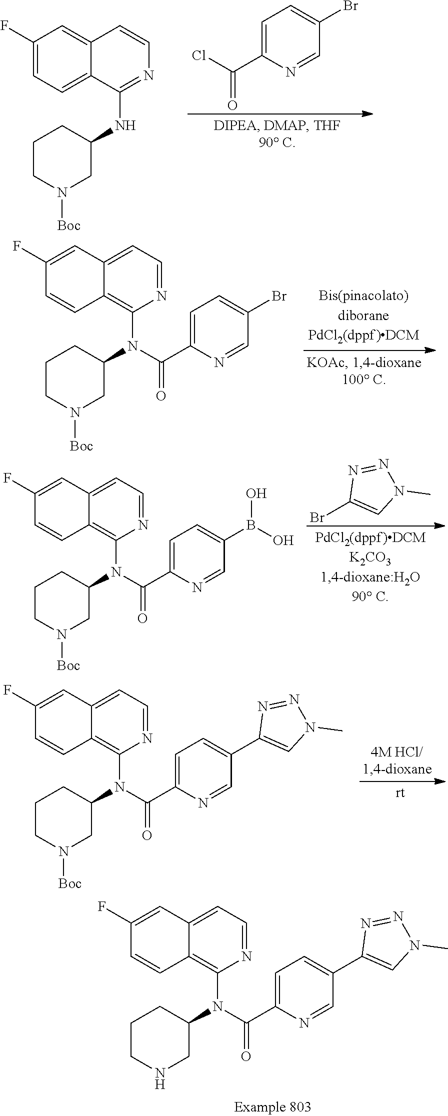

### Step 1:

To a solution of tert-butyl (R)-3-((6-fluoroisoquinolin-1-yl) amino) piperidine-1-carboxylate (500 mg, 1.448 mmol) in THF (20 mL) at 0° C. were added DIPEA (1.011 mL, 5.79 mmol), DMAP (18 mg, 0.145 mmol), and 5-bromopicolinoyl chloride (638 mg, 2.90 mmol). The reaction was stirred at 90° C. for 16 h in a sealed tube. The reaction mixture was diluted with water, and the product was extracted with EtOAc (2×50 mL). The combined organic layer was washed with brine, dried over Na₂SO₄, and concentrated. The obtained compound was purified by Combi-flash using 60% EtOAc in hexane to afford tert-butyl (R)-3-(5-bromo-N-(6-fluoroisoquinolin-1-yl)picolinamido)piperidine-1-carboxylate (600 mg, 78%) as a brown solid.

### Step 2:

A stirred solution of tert-butyl (R)-3-(5-bromo-N-(6-fluoroisoquinolin-1-yl)picolinamido)piperidine-1-carboxylate (600 mg, 1.133 mmol), bis(pinacolato)diborane (863 mg, 3.40 mmol), and K₂CO₃ (334 mg, 3.40 mmol) in 1,4-dioxane (10 mL) was purged with nitrogen for 10 minutes. PdCl₂(dppf)-CH₂Cl₂ adduct (46.3 mg, 0.057 mmol) was then added to the reaction mixture, and the reaction was stirred at 100° C. for 16 h. The reaction mixture was diluted with EtOAc (50 mL) and filtered through a celite pad. The filtrate was washed with water and brine, and the organic layer was dried over Na₂SO₄ and concentrated under reduced pressure to afford (R)-(6-((1-(tert-butoxycarbonyl) piperidin-3-yl) (6-fluoroisoquinolin-1-yl)carbamoyl) pyridin-3-yl) boronic acid (600 mg, crude) as brown gummy solid. LC-MS: [M+H⁺]=495.1.

### Step 3:

A stirred solution of (R)-(6-((1-(tert-butoxycarbonyl) piperidin-3-yl) (6-fluoroisoquinolin-1-yl) carbamoyl) pyridin-3-yl) boronic acid (600 mg, 1.214 mmol), K₂CO₃ (0.335 g, 2.428 mmol) in 1,4-dioxane (10 mL) and water (4 mL) was purged with nitrogen for 10 minutes. PdCl₂(dppf)-CH₂Cl₂ adduct (99 mg, 0.121 mmol) was added to the reaction, and the reaction mixture was stirred at 90° C. for 16 h in a sealed tube. The reaction mixture was diluted with EtOAc (100 mL) and filtered through a celite pad. The filtrate was washed with water and brine, and the organic layer was dried over Na₂SO₄ and concentrated. The obtained compound was purified by Combi-flash using 100% EtOAc. The obtained compound was further purified by prep HPLC (Conditions: X-SELECT CSH, Mobile phase A: 0.1% formic acid in water (100%), Mobile phase B: 100% MeCN, Gradient: T/% B: 0/55, 10/80, Flow: 15 mL/min, Dilution: MeCN+Water). Pure fractions were dried under vacuum to afford tert-butyl (R)-3-(N-(6-fluoroisoquinolin-1-yl)-5-(1-methyl-1H-1,2,3-triazol-4-yl)picolinamido)piperidine-1-carboxylate (200 mg, 65.3%) as an off-white solid.

### Step 4:

Example 803 was prepared by the deprotecting procedure described for Example 97, provided the title compound hydrochloride salt as an off-white solid. LC-MS: [M+H⁺]=432.1 ¹H NMR (CD₃OD, 400 MHZ): δ 8.43-8.42 (m, 1H), 8.37-8.28 (m, 2H), 8.22-8.17 (m, 1H), 8.13-8.10 (m, 1H), 7.98-7.83 (m, 2H), 7.66-7.57 (m, 1H), 7.49-7.45 (m, 1H), 5.19-5.13 (m, 1H), 4.08 (s, 3H), 3.94-3.91 (m, 1H), 3.73-3.67 (m, 1H), 3.53-3.46 (m, 1H), 2.96-2.84 (m, 1H), 2.12-1.84 (m, 3H), 1.45-1.41 (m, 1H).

### Synthesis of (R)—N-(6-fluoroisoquinolin-1-yl)-4-(5-methyl-1,3,4-thiadiazol-2-yl)-N-(piperidin-3-yl)benzamide (Example 804)

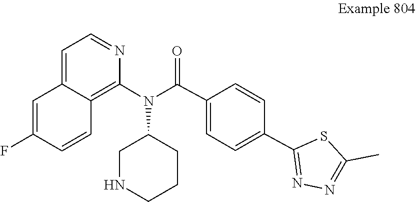 [SMILES: Cc1nnc(-c2ccc(C(=O)N(c3nccc4cc(F)ccc34)[C@@H]3CCCNC3)cc2)s1]

tert-butyl (R)-3-((6-fluorooisoquinolin-1-yl)amino)piperidine-1-carboxylate was prepared according to the procedure described for tert-butyl (R)-3-((8-methylisoquinolin-1-yl)amino)piperidine-1-carboxylate by substituting 1-chloro-8-methylisoquinoline with 1-chloro-6-fluoroisoquinoline.

Example 804 was prepared according to the procedure described for Example 444 by substituting tert-butyl (3R)-3-[(6-chloro-1-isoquinolyl)amino]piperidine-1-carboxylate and 2-fluoro-4-(1-methyltriazol-4-yl)benzoyl chloride with tert-butyl (R)-3-((6-fluorooisoquinolin-1-yl)amino)piperidine-1-carboxylate and 4-(5-methyl-1, 3, 4-thiadiazol-2-yl) benzoyl chloride, provided the title compound hydrochloride salt as a white solid. LC-MS: [M+H⁺]=448.2. ¹H NMR (400 MHZ, CD₃OD): δ ppm 8.50 (br d, J=5.6 Hz, 1H), 8.13 (br dd, J=5.2, 8.7 Hz, 1H), 7.79 (br d, J=5.6 Hz, 1H), 7.62-7.51 (m, 3H), 7.45 (br t, J=8.1 Hz, 1H), 7.34 (br d, J=7.5 Hz, 2H), 5.34-5.17 (m, 1H), 3.92 (br d, J=11.4 Hz, 1H), 3.81-3.64 (m, 1H), 3.35 (br d, J=4.0 Hz, 1H), 2.99-2.81 (m, 1H), 2.74 (s, 3H), 2.06-1.77 (m, 3H), 1.43-1.18 (m, 1H).

### Synthesis of (R)—N-(6-fluoroisoquinolin-1-yl)-4-(1-methyl-1H-1,2,3-triazol-4-yl)-N-(piperidin-3-yl)benzamide (Example 805)

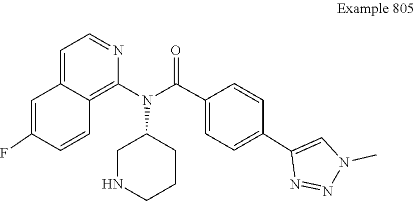 [SMILES: Cn1cc(-c2ccc(C(=O)N(c3nccc4cc(F)ccc34)[C@@H]3CCCNC3)cc2)nn1]

Example 805 was prepared according to the procedure described for Example 444 by substituting tert-butyl (3R)-3-[(6-chloro-1-isoquinolyl)amino]piperidine-1-carboxylate and 2-fluoro-4-(1-methyltriazol-4-yl)benzoyl chloride with tert-butyl (R)-3-((6-fluorooisoquinolin-1-yl)amino)piperidine-1-carboxylate and 4-(1-methyltriazol-4-yl) benzoyl chloride, provided the title compound hydrochloride salt as a white solid. LC-MS: [M+H⁺]=431.3. ¹H NMR (400 MHz, CD₃OD): δ ppm 8.51 (br d, J=5.3 Hz, 1H), 8.24-8.03 (m, 2H), 7.79 (d, J=5.8 Hz, 1H), 7.64-7.40 (m, 4H), 7.29 (br s, 2H), 5.41-5.17 (m, 1H), 4.09 (s, 3H), 3.92 (br d, J=10.6 Hz, 1H), 3.81-3.68 (m, 1H), 3.38 (br s, 1H), 2.89 (br s, 1H), 2.16-1.81 (m, 3H), 1.34 (br d, J=6.5 Hz, 1H).

### Synthesis of N-(6-chloro-1-isoquinolyl)-N-[(3R)-3-piperidyl]-5-pyridazin-3-yl-pyridine-2-carboxamide (Example 806)

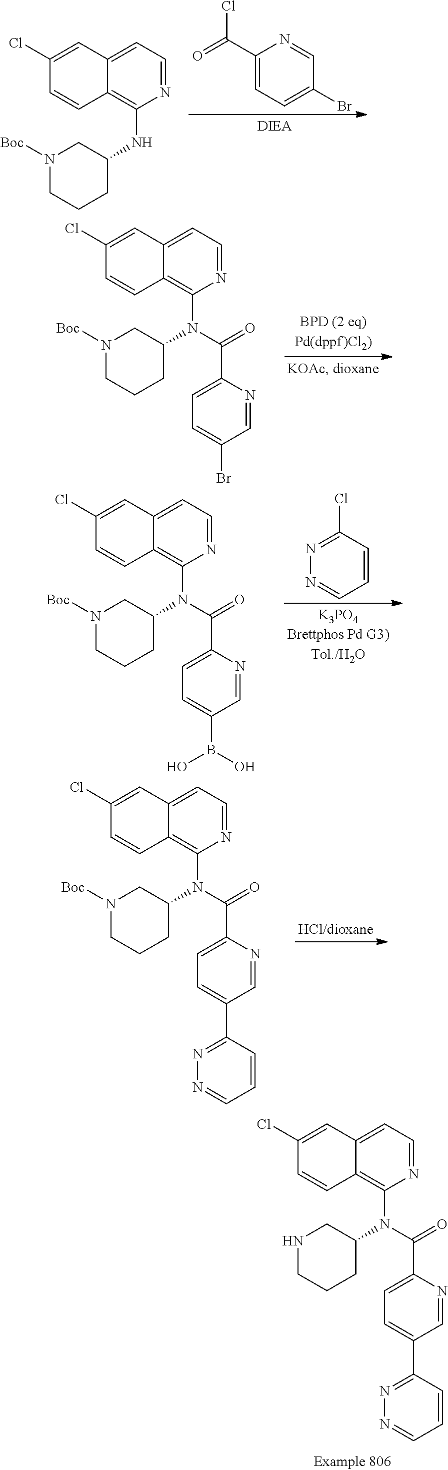

tert-butyl (R)-3-((6-chloroisoquinolin-1-yl)amino)piperidine-1-carboxylate was prepared according to the procedure described for tert-butyl (R)-3-((8-methylisoquinolin-1-yl)amino)piperidine-1-carboxylate by substituting 1-chloro-8-methylisoquinoline with 1,6-dichloroisoquinoline.

Example 806 was prepared according to the procedure described for Example 701 by substituting tert-butyl (R)-3-((6,8-dimethylisoquinolin-1-yl)amino)piperidine-1-carboxylate and 2-bromo-5-methyl-1,3,4-thiadiazole with tert-butyl (R)-3-((6-chloroisoquinolin-1-yl)amino)piperidine-1-carboxylate and 3-chloropyridazine, provided the title compound hydrochloride salt as an off white solid. LC-MS: [M+H⁺]=445.2. ¹H NMR (400 MHZ, DMSO-d6): δ ppm 9.54-9.15 (m, 2H), 9.09-8.73 (m, 1H), 8.66-8.38 (m, 3H), 8.33-8.08 (m, 3H), 7.98-7.86 (m, 1H), 7.85-7.70 (m, 2H), 7.62 (dd, J=1.5, 9.0 Hz, 1H), 5.14-4.58 (m, 1H), 3.81-3.71 (m, 1H), 3.45-3.37 (m, 1H), 3.25-3.11 (m, 1H), 2.84-2.62 (m, 1H), 2.01-1.65 (m, 1H), 1.97-1.59 (m, 2H), 1.34-1.13 (m, 1H).

### Synthesis of (R)-4-(3H-[1,2,3]triazolo[4,5-b]pyridin-3-yl)-2-fluoro-N-(piperidin-3-yl)-N-(2-(1,2,3,6-tetrahydropyridin-4-yl)thieno[3,2-c]pyridin-4-yl)benzamide (Example 807)

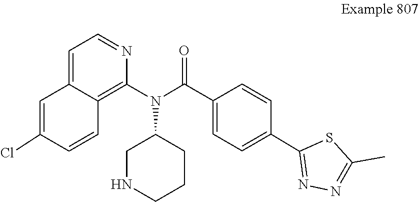 [SMILES: Cc1nnc(-c2ccc(C(=O)N(c3nccc4cc(Cl)ccc34)[C@@H]3CCCNC3)cc2)s1]

Example 807 was prepared according to the procedure described for Example 444 by substituting tert-butyl (3R)-3-[(6-chloro-1-isoquinolyl)amino]piperidine-1-carboxylate and 2-fluoro-4-(1-methyltriazol-4-yl)benzoyl chloride with tert-butyl (R)-3-((6-chloroisoquinolin-1-yl)amino)piperidine-1-carboxylate and 4-(5-methyl-1,3,4-thiadiazol-2-yl)benzoyl chloride, provided the title compound as a white solid. LC-MS: [M+H]=464.1. ¹H NMR (400 MHZ, DMSO-d₆): δ ppm 8.59-8.38 (m, 1H), 8.20-7.96 (m, 2H), 7.85-7.48 (m, 4H), 7.27 (br s, 2H), 4.78-4.47 (m, 1H), 3.08-2.87 (m, 1H), 2.70 (br s, 4H), 2.27-2.09 (m, 2H), 1.92-1.37 (m, 3H), 0.98 (br d, J=9.6 Hz, 1H).

### Synthesis of (R)—N-(6-chloroisoquinolin-1-yl)-4-(1-methyl-1H-1,2,3-triazol-4-yl)-N-(piperidin-3-yl)benzamide (Example 808)

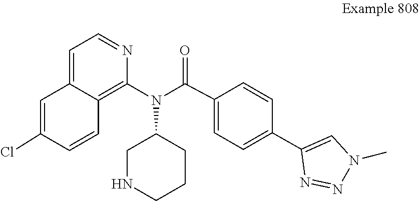 [SMILES: Cn1cc(-c2ccc(C(=O)N(c3nccc4cc(Cl)ccc34)[C@@H]3CCCNC3)cc2)nn1]

Example 808 was prepared according to the procedure described for Example 444 by substituting tert-butyl (3R)-3-[(6-chloro-1-isoquinolyl)amino]piperidine-1-carboxylate and 2-fluoro-4-(1-methyltriazol-4-yl)benzoyl chloride with tert-butyl (R)-3-((6-chloroisoquinolin-1-yl)amino)piperidine-1-carboxylate and 4-(1-methyl-1H-1, 2, 3-triazol-4-yl) benzoyl chloride, provided the title compound hydrochloride salt as a white solid. LC-MS: [M+H⁺]=447.2. ¹H NMR (400 MHZ, CD₃OD): δ ppm 8.51 (br d, J=3.8 Hz, 1H), 8.26 (s, 1H), 8.03 (br d, J=7.3 Hz, 1H), 7.90 (br s, 1H), 7.75 (d, J=5.8 Hz, 1H), 7.61 (br s, 1H), 7.48 (br d, J=7.3 Hz, 2H), 7.31 (br s, 2H), 5.47-5.09 (m, 1H), 4.11 (s, 3H), 3.99-3.61 (m, 2H), 3.35 (br d, J=13.0 Hz, 1H), 2.87 (br s, 1H), 2.16-1.79 (m, 3H), 1.31 (br dd, J=7.4, 13.3 Hz, 1H).

### Synthesis of (R)-5-(1-methyl-1H-1,2,3-triazol-4-yl)-N-(8-methylisoquinolin-1-yl)-N-(piperidin-3-yl)picolinamide (Example 809)

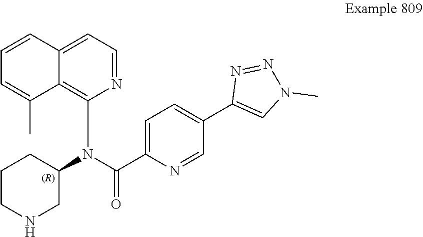

Example 809 was prepared according to the procedure described for Example 701 by substituting tert-butyl (R)-3-((6,8-dimethylisoquinolin-1-yl)amino)piperidine-1-carboxylate and 2-bromo-5-methyl-1,3,4-thiadiazole with tert-butyl (R)-3-((8-methylisoquinolin-1-yl)amino)piperidine-1-carboxylate and 4-bromo-1-methyl-1H-1,2,3-triazole, provided the title compound hydrochloride salt as a pale yellow solid. LC-MS: [M+H⁺]=428.2. ¹H NMR (CD₃OD, 400 MHZ): δ ppm 8.49-8.47 (m, 1H), 8.38-8.36 (m, 1H), 8.33-8.29 (m, 2H), 8.29-8.19 (m, 1H), 8.19-8.17 (m, 1H), 8.13-8.11 (m, 1H), 7.95-7.93 (m, 1H), 7.74-7.72 (m, 1H), 5.07-4.97 (m, 1H), 4.09 (s, 3H), 4.08-4.06 (m, 1H), 3.52-3.46 (m, 1H), 2.95-2.82 (m, 2H), 2.73 (s, 3H), 1.89 (bs, 2H), 1.72-1.69 (m, 1H), 1.06-1.02 (m, 1H).

### Synthesis of (R)-5-(5-methyl-1,3,4-thiadiazol-2-yl)-N-(3-methylthieno[3,2-c]pyridin-4-yl)-N-(piperidin-3-yl)picolinamide (Example 810)

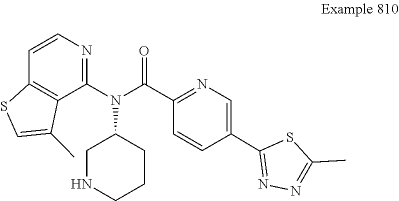 [SMILES: Cc1nnc(-c2ccc(C(=O)N(c3nccc4scc(C)c34)[C@@H]3CCCNC3)nc2)s1]

Example 810 was prepared according to the procedure described for Example 701 by substituting tert-butyl (R)-3-((6,8-dimethylisoquinolin-1-yl)amino)piperidine-1-carboxylate with tert-butyl (R)-3-((3-methylthieno[3,2-c]pyridin-4-yl)amino)piperidine-1-carboxylate, provided the title compound as a white solid. LC-MS: [M+H]=451.1. ¹H NMR (400 MHZ, DMSO-d₆): δ ppm 0.76-0.97 (m, 1H) 1.41-1.80 (m, 3H) 1.95-2.29 (m, 3H) 2.53-2.58 (m, 3H) 2.72-2.77 (m, 3H) 2.93-3.10 (m, 2H) 4.22-4.61 (m, 1H) 7.42-7.50 (m, 1H) 7.53-7.65 (m, 1H) 7.85-8.00 (m, 1H) 8.09-8.22 (m, 1H) 8.23-8.34 (m, 1H) 8.48-8.59 (m, 1H).

### Synthesis of (R)-5-(1-methyl-1H-1,2,3-triazol-4-yl)-N-(3-methylthieno[3,2-c]pyridin-4-yl)-N-(piperidin-3-yl)picolinamide (Example 811)

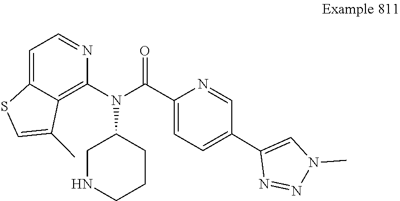 [SMILES: Cc1csc2ccnc(N(C(=O)c3ccc(-c4cn(C)nn4)cn3)[C@@H]3CCCNC3)c12]

Example 811 was prepared according to the procedure described for Example 699 by substituting 2-fluoro-4-iodobenzoyl chloride and 2-bromo-5-methyl-1,3,4-oxadiazole with 5-bromopicolinoyl chloride and 4-bromo-1-methyl-1H-1,2,3-triazole, provided the title compound hydrochloride salt as a yellow solid. LC-MS: [M+H]=434.3. ¹H NMR (400 MHZ, CD₃OD): δ ppm 1.12-1.37 (m, 1H) 1.72-1.97 (m, 3H) 2.41-2.60 (m, 3H) 2.81-3.03 (m, 1H) 3.51-3.77 (m, 2H) 3.97 (br d, J=11.38 Hz, 1H) 4.10 (s, 3H) 5.02-5.18 (m, 1H) 7.45-7.59 (m, 1H) 7.87 (br d, J=8.25 Hz, 1H) 8.12-8.23 (m, 2H) 8.31 (s, 1H) 8.38-8.47 (m, 2H).

### Synthesis of (R)—N-(6-chloro-8-methylisoquinolin-1-yl)-5-(1-methyl-1H-1,2,3-triazol-4-yl)-N-(piperidin-3-yl)picolinamide (Example 812)

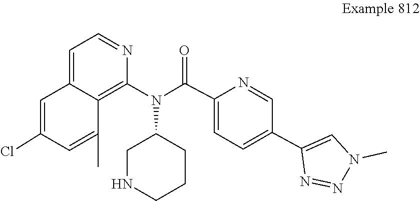 [SMILES: Cc1cc(Cl)cc2ccnc(N(C(=O)c3ccc(-c4cn(C)nn4)cn3)[C@@H]3CCCNC3)c12]

Example 812 was prepared according to the procedure described for Example 816 by substituting 4-bromo-2-chlorobenzoyl chloride with 5-bromopicolinoyl chloride, provided the title compound hydrochloride salt as a white solid. LC-MS: [M+H⁺]=462.4. ¹H NMR: (400 MHz, CD₃OD): δ ppm 8.39-8.33 (m, 1H), 8.33-8.24 (m, 2H), 8.22-8.14 (m, 1H), 8.01-7.79 (m, 3H), 7.58-7.49 (m, 1H), 5.04-4.95 (m, 1H), 4.09 (s, 3H), 4.00 (br d, J=11.8 Hz, 1H), 3.53 (br t, J=11.4 Hz, 1H), 3.32 (br s, 1H), 2.98-2.83 (m, 2H), 2.78 (s, 3H), 1.96-1.78 (m, 3H), 1.21-1.05 (m, 1H).

### Synthesis of (R)-4-(2-fluoro-4-(1-methyl-1H-1,2,3-triazol-4-yl)-N-(piperidin-3-yl)benzamido)-N-(pyridin-4-yl)thieno[3,2-c]pyridine-2-carboxamide (Example 813)

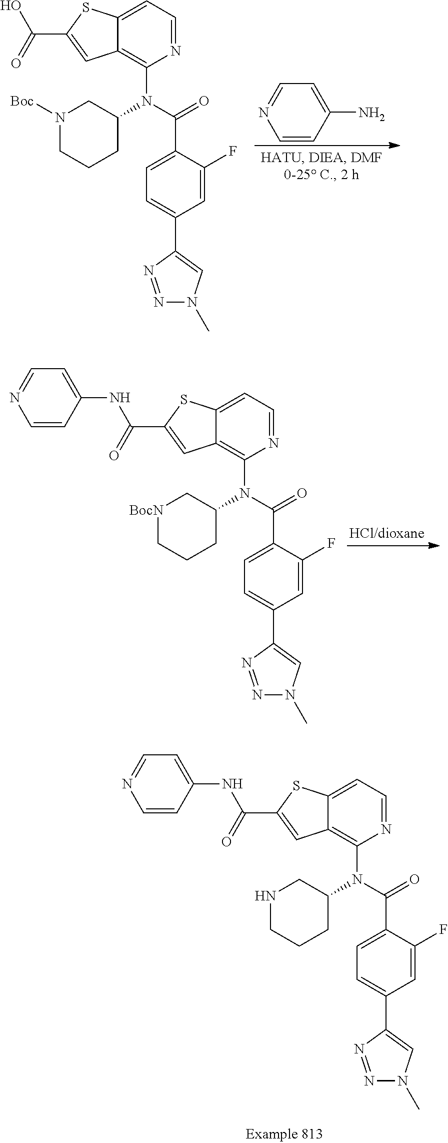 [SMILES: Cn1cc(-c2ccc(C(=O)N(c3nccc4sc(C(=O)O)cc34)[C@@H]3CCCN(C(=O)OC(C)(C)C)C3)c(F)c2)nn1 ; Nc1ccncc1 ; Cn1cc(-c2ccc(C(=O)N(c3nccc4sc(C(=O)Nc5ccncc5)cc34)[C@@H]3CCCN(C(=O)OC(C)(C)C)C3)c(F)c2)nn1 ; Cn1cc(-c2ccc(C(=O)N(c3nccc4sc(C(=O)Nc5ccncc5)cc34)[C@@H]3CCCNC3)c(F)c2)nn1]

### Step 1:

To a solution of (R)-4-(N-(1-(tert-butoxycarbonyl)piperidin-3-yl)-2-fluoro-4-(1-methyl-1H-1,2,3-triazol-4-yl)benzamido)thieno[3,2-c]pyridine-2-carboxylic acid (40 mg, 68.89 μmol) in THF (1 mL) was added DIEA (26.71 mg, 206.67 μmol), pyridin-4-amine (19.45 mg, 206.67 μmol) and HATU (31.43 mg, 82.67 μmol). The mixture was stirred at 25° C. for 2 h. The reaction mixture was concentrated under reduced pressure. The residue was purified by prep-HPLC. (column: Phenomenex luna C18 100*40 mm*5 μm; mobile phase: [H₂O (0.04% HCl)-MeCN]; gradient: 20%-55% B over 8.0 min) to give tert-butyl (R)-3-(2-fluoro-4-(1-methyl-1H-1,2,3-triazol-4-yl)-N-(2-(pyridin-4-ylcarbamoyl)thieno[3,2-c]pyridin-4-yl)benzamido)piperidine-1-carboxylate (10 mg, 22%) as a yellow solid.

### Step 2:

The title compound hydrochloride salt was obtained as an off-white solid by deprotection Method A used in Example 97. LC-MS: [M+H⁺]=557.3. ¹H NMR (400 MHZ, CD₃OD): δ ppm 8.76 (br d, J=6.0 Hz, 2H), 8.33 (br d, J=4.9 Hz, 1H), 8.08 (br d, J=6.9 Hz, 2H), 7.94 (br s, 1H), 7.77 (br d, J=4.3 Hz, 1H), 7.51 (br d, J=5.5 Hz, 1H), 7.33 (br d, J=10.0 Hz, 1H), 7.21 (br s, 2H), 5.24-5.11 (m, 1H), 4.83-4.78 (m, 1H), 4.20 (s, 3H), 3.89-3.80 (m, 1H), 3.62-3.51 (m, 1H), 3.30-3.26 (m, 2H), 2.90-2.77 (m, 1H), 2.12-1.78 (m, 3H), 1.38-1.18 (m, 1H).

### Synthesis of (R)—N-(6-chloro-8-methylisoquinolin-1-yl)-4-(1,5-dimethyl-1H-pyrazol-4-yl)-2-fluoro-N-(piperidin-3-yl)benzamide (Example 814)

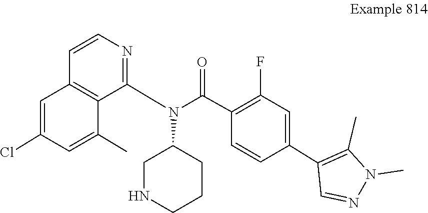 [SMILES: Cc1cc(Cl)cc2ccnc(N(C(=O)c3ccc(-c4cnn(C)c4C)cc3F)[C@@H]3CCCNC3)c12]

Example 814 was prepared according to the procedure described for Example 816 by substituting 4-bromo-2-chloro-benzoyl chloride with 4-bromo-2-fluoro-benzoyl chloride and substituting 4-bromo-1-methyl-triazole with 4-bromo-1,5-dimethyl-1H-1,2,3-triazole, provided the title compound hydrochloride salt as a light solid. LC-MS: [M+H]=492.3. ¹H NMR (400 MHZ, DMSO-d₆): δ ppm 9.10-8.88 (m, 2H), 8.52-8.43 (m, 1H), 7.92-7.86 (m, 1H), 7.81-7.74 (m, 1H), 7.55 (d, J=1.1 Hz, 1H), 7.50-7.45 (m, 1H), 6.97-6.86 (m, 1H), 6.79-6.72 (m, 2H), 5.01-4.71 (m, 1H), 3.82-3.66 (m, 4H), 3.36-3.30 (m, 1H), 2.94-2.72 (m, 5H), 2.19 (s, 3H), 1.91-1.70 (m, 3H), 1.42-1.24 (m, 1H).

### Synthesis of (R)—N-(6-chloro-8-methylisoquinolin-1-yl)-2-fluoro-4-(5-methyl-1,3,4-thiadiazol-2-yl)-N-(piperidin-3-yl)benzamide (Example 815)

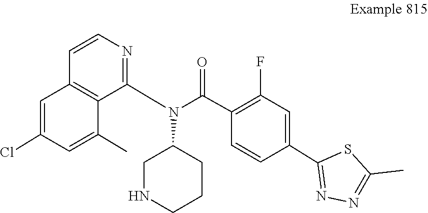 [SMILES: Cc1nnc(-c2ccc(C(=O)N(c3nccc4cc(Cl)cc(C)c34)[C@@H]3CCCNC3)c(F)c2)s1]

Example 815 was prepared according to the procedure described for Example 816 by substituting 4-bromo-2-chloro-benzoyl chloride and 4-bromo-1-methyl-triazole with 2-fluoro-4-iodo-benzoyl chloride and 2-bromo-5-methyl-1, 3, 4-thiadiazole, provided the title compound as a white solid. LC-MS: [M+H⁺]=496.2. ¹H NMR (400 MHZ, CD₃OD): δ ppm 8.45-8.36 (m, 1H), 7.70-7.57 (m, 2H), 7.45 (s, 1H), 7.39 (br d, J=10.6 Hz, 1H), 7.27 (d, J=8.0 Hz, 1H), 7.06-6.93 (m, 1H), 4.76-4.52 (m, 1H), 3.71 (br d, J=11.8 Hz, 1H), 3.31-3.27 (m, 1H), 2.98 (br d, J=16.4 Hz, 1H), 2.87-2.82 (m, 3H), 2.73 (s, 3H), 2.56-2.43 (m, 1H), 1.96-1.60 (m, 3H), 1.44-1.25 (m, 1H).

### Synthesis of (R)-2-chloro-N-(6-chloro-8-methylisoquinolin-1-yl)-4-(1-methyl-1H-1,2,3-triazol-4-yl)-N-(piperidin-3-yl)benzamide (Example 816)

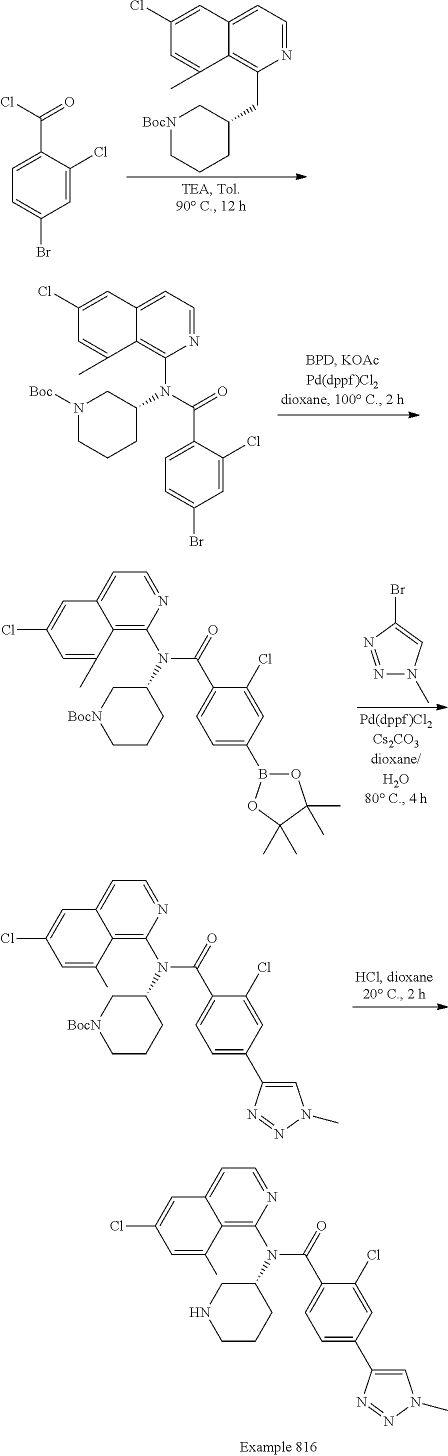

### Step 1:

To a solution of tert-butyl (R)-3-((6-chloro-8-methylisoquinolin-1-yl)amino)piperidine-1-carboxylate (500 mg, 1.33 mmol) and TEA (403.80 mg, 3.99 mmol, 555.43 μL) in toluene (5 mL) was added 4-bromo-2-chloro-benzoyl chloride (1.01 g, 3.99 mmol) at 20° C. under N₂ atmosphere. The mixture was heated to 90° C. and stirred for 12 h. The reaction mixture was quenched by addition methanol (10 mL) at 25° C. and concentrated. The residue was purified by column chromatography (SiO₂, petroleum ether/EtOAc=8/1 to 4/1) to give tert-butyl (R)-3-(4-bromo-2-chloro-N-(6-chloro-8-methylisoquinolin-1-yl)benzamido)piperidine-1-carboxylate (522 mg, 66%) as a white solid. LC-MS: [M+H⁺]=594.2.

### Step 2:

To a solution of tert-butyl (3R)-3-[(4-bromo-2-chloro-benzoyl)-(6-chloro-8-methyl-1-isoquinolyl) amino] piperidine-1-carboxylate (520 mg, 876.40 μmol), KOAc (258.03 mg, 2.63 mmol) and bis(pinacolato)diboron (445.10 mg, 1.75 mmol) in dioxane (5 mL) was added Pd(dppf)Cl₂ (64.13 mg, 87.64 μmol) at 20° C. under N₂ atmosphere. The mixture was heated to 100° C. and stirred for 2 h. The reaction mixture was filtered and concentrated. The reaction mixture was filtered and concentrated under reduced pressure to give tert-butyl (3R)-3-[(6-chloro-8-methyl-1-isoquinolyl)-[2-chloro-4-(4, 4, 5, 5-tetramethyl-1, 3, 2-dioxaborolan-2-yl) benzoyl] amino] piperidine-1-carboxylate (525 mg, 93%) as a white solid. LC-MS: [M+H⁺]=641.4.

### Step 3:

A mixture of tert-butyl (3R)-3-[(6-chloro-8-methyl-1-isoquinolyl)-[2-chloro-4-(4,4,5,5-tetramethyl-1,3,2-dioxaborolan-2-yl) benzoyl]amino]piperidine-1-carboxylate (150 mg, 234.23 μmol), 4-bromo-1-methyl-triazole (56.91 mg, 351.34 μmol), Cs₂CO₃ (228.95 mg, 702.68 μmol) in dioxane (2 mL) and H₂O (0.4 mL) was added Pd(dppf)Cl₂ (17.14 mg, 23.42 μmol) at 20° C. under N₂ atmosphere. The mixture was heated to 80° C. and stirred for 4 h under N₂ atmosphere. The reaction mixture was filtered and concentrated under reduced pressure to give a residue. The residue was purified by prep-TLC (SiO₂, EtOAc/Petroleum ether=2:1) to give tert-butyl (3R)-3-[(6-chloro-8-methyl-1-isoquinolyl)-[2-chloro-4-(1-methyltriazol-4-yl) benzoyl] amino] piperidine-1-carboxylate (93 mg, 66%) was obtained as a yellow solid. LC-MS: [M+H⁺]=596.5.

### Step 4:

To a solution of tert-butyl (3R)-3-[(6-chloro-8-methyl-1-isoquinolyl)-[2-chloro-4-(1-methyltriazol-4-yl) benzoyl] amino] piperidine-1-carboxylate (80 mg, 134.34 μmol) in dioxane (1 mL) was added 4 M HCl/dioxane (1.80 mL). The mixture was stirred at 20° C. for 2 h. The reaction mixture was concentrated under reduced pressure to give the title compound hydrochloride salt (57 mg, 76%) as a yellow solid. LC-MS: [M+H⁺]=495.3. ¹H NMR (400 MHz, CD₃OD): δ ppm 8.38 (d, J=5.5 Hz, 1H), 8.17 (s, 1H), 7.73-7.65 (m, 1H), 7.64-7.60 (m, 1H), 7.60-7.55 (m, 1H), 7.55-7.50 (m, 1H), 7.29-7.17 (m, 1H), 7.01-6.86 (m, 1H), 5.12-4.98 (m, 1H), 4.07 (s, 3H), 3.87-3.67 (m, 1H), 3.48-3.36 (m, 1H), 3.11-2.97 (m, 1H), 2.96-2.88 (m, 3H), 2.84-2.66 (m, 1H), 2.24-1.83 (m, 3H), 1.69-1.49 (m, 1H).

### Synthesis of N-[2-(5-amino-5-oxo-pent-1-ynyl)thieno[3,2-c]pyridin-4-yl]-2-fluoro-4-(5-methyl-1,3,4-thiadiazol-2-yl)-N-[(3R)-3-piperidyl]benzamide (Example 817)

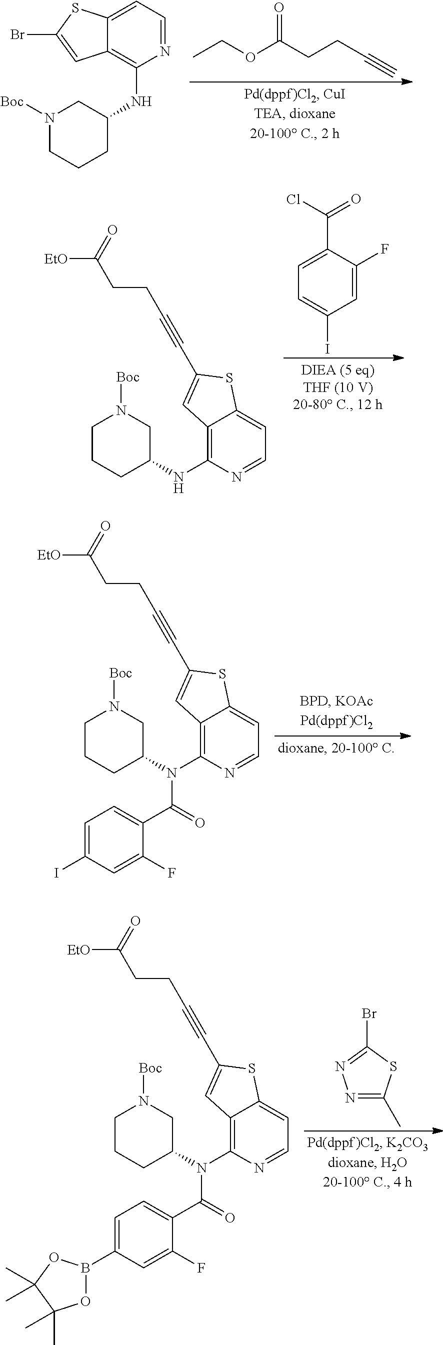

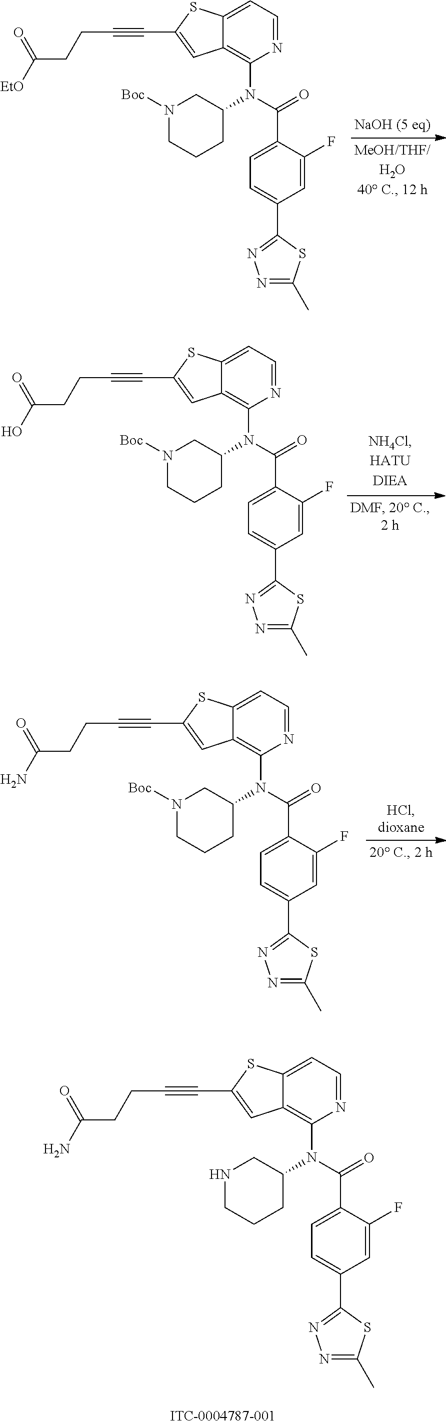 [SMILES: CCOC(=O)CCC#Cc1cc2c(N(C(=O)c3ccc(-c4nnc(C)s4)cc3F)[C@@H]3CCCN(C(=O)OC(C)(C)C)C3)nccc2s1 ; Cc1nnc(-c2ccc(C(=O)N(c3nccc4sc(C#CCCC(=O)O)cc34)[C@@H]3CCCN(C(=O)OC(C)(C)C)C3)c(F)c2)s1 ; Cc1nnc(-c2ccc(C(=O)N(c3nccc4sc(C#CCCC(N)=O)cc34)[C@@H]3CCCN(C(=O)OC(C)(C)C)C3)c(F)c2)s1 ; Cc1nnc(-c2ccc(C(=O)N(c3nccc4sc(C#CCCC(N)=O)cc34)[C@@H]3CCCNC3)c(F)c2)s1]

### Step 1:

To a mixture of tert-butyl (3R)-3-[(2-bromothieno[3,2-c]pyridin-4-yl)amino]piperidine-1-carboxylate (0.8 g, 1.94 mmol, 1 eq) and ethyl pent-4-ynoate (1.22 g, 9.70 mmol, 5 eq) in TEA (8 mL) and dioxane (8 mL) was added Pd(dppf)Cl₂ (141.96 mg, 194.01 μmol, 0.1 eq) and CuI (36.95 mg, 194.01 μmol, 0.1 eq) under N₂ atmosphere at 20° C. The suspension was degassed and purged with N₂ for 3 times. The mixture was stirred under N₂ at 100° C. for 2 hours. The reaction mixture was diluted with water (20 mL) and extracted with EtOAc (3×20 mL). The combined organic layer was washed with brine (20 mL), dried over Na₂SO₄, filtered and concentrated under reduced pressure to give a residue. The residue was purified by flash column using 90% EtOAc in petroleum ether to give (3R)-3-[[2-(5-ethoxy-5-oxo-pent-1-ynyl)thieno[3,2-c]pyridin-4-yl]amino]piperidine-1-carboxylate (1.05 g, 96%) as a yellow oil. LC-MS: [M+H]=458.1.

### Step 2:

To a solution of tert-butyl (3R)-3-[[2-(5-ethoxy-5-oxo-pent-1-ynyl)thieno[3,2-c]pyridin-4-yl]amino]piperidine-1-carboxylate (0.55 g, 781.28 μmol, 1 eq) 2-fluoro-4-iodo-benzoyl chloride (452.16 mg, 1.56 mmol, 2 eq) in THF (5 mL) was added DIEA (395.25 mg, 3.06 mmol, 532.68 μL, 3.91 eq) at 20° C. Then the mixture was stirred at 80° C. for 2 hours. The reaction mixture was diluted with water (10 mL) and extracted with EtOAc (3×10 mL). The combined organic layer was washed with brine (10 mL), dried over Na₂SO₄, filtered and concentrated under reduced pressure to give a residue. The residue was purified by prep-TLC using 60% EtOAc in petroleum ether to give tert-butyl (3R)-3-[[2-(5-ethoxy-5-oxo-pent-1-ynyl)thieno[3,2-c]pyridin-4-yl]-(2-fluoro-4-iodo-benzoyl)amino]piperidine-1-carboxylate (0.8 g, 95%) as a yellowish oil.

### Step 3:

To a mixture of tert-butyl (3R)-3-[[2-(5-ethoxy-5-oxo-pent-1-ynyl)thieno[3,2-c]pyridin-4-yl]-(2-fluoro-4-iodo-benzoyl)amino]piperidine-1-carboxylate (0.4 g, 340.15 μmol, 1 eq) in dioxane (4 mL) was added bis(pinacolato)diboron (259.13 mg, 1.02 mmol, 3 eq), KOAc (100.15 mg, 1.02 mmol, 3 eq) and Pd(dppf)Cl₂ (24.89 mg, 34.01 μmol, 0.1 eq) in order at 20° C. under N₂ atmosphere. The suspension was degassed and purged with N₂ for 3 times. The mixture was stirred under N₂ at 100° C. for 12 hours. The reaction mixture was concentrated. The residue was purified by prep-TLC using 30% EtOAc in petroleum ether to give tert-butyl (3R)-3-[[2-(5-ethoxy-5-oxo-pent-1-ynyl)thieno[3,2-c]pyridin-4-yl]-[2-fluoro-4-(4,4,5,5-tetramethyl-1,3,2-dioxaborolan-2-yl)benzoyl]amino]piperidine-1-carboxylate (0.35 g, 58%) as a brown oil.

### Step 4:

To a mixture of 2-bromo-5-methyl-1,3,4-thiadiazole (68.03 mg, 379.97 μmol, 1.5 eq) tert-butyl (3R)-3-[[2-(5-ethoxy-5-oxo-pent-1-ynyl)thieno [3,2-c]pyridin-4-yl]-[2-fluoro-4-(4,4,5,5-tetramethyl-1,3,2-dioxaborolan-2-yl)benzoyl]amino]piperidine-1-carboxylate (0.275 g, 253.32 μmol) in dioxane (3 mL) and H₂O (0.6 mL) was added K₂CO₃ (105.03 mg, 759.95 μmol, 3 eq) and Pd(dppf)Cl₂ (18.54 mg, 25.33 μmol) at 20° C. under N₂ atmosphere. The suspension was degassed and purged with N₂ for 3 times. The mixture was stirred under N₂ at 100° C. for 2 hours. The reaction mixture was diluted with water (10 mL) and extracted with EtOAc (3×10 mL). The combined organic layer was washed with brine, dried over Na₂SO₄, filtered and concentrated. The residue was purified by prep-TLC using 30% EtOAc in petroleum ether to give tert-butyl (3R)-3-[[2-(5-ethoxy-5-oxo-pent-1-ynyl)thieno[3,2-c]pyridin-4-yl]-[2-fluoro-4-(5-methyl-1,3,4-thiadiazol-2-yl)benzoyl]amino]piperidine-1-carboxylate (0.26 g, 82%) as a yellow oil.

### Step 5:

To a solution of tert-butyl (3R)-3-[[2-(5-ethoxy-5-oxo-pent-1-ynyl)thieno[3,2-c]pyridin-4-yl]-[2-fluoro-4-(5-methyl-1,3,4-thiadiazol-2-yl)benzoyl]amino]piperidine-1-carboxylate (0.21 g, 263.35 μmol, 1 eq) in THF (0.8 mL), MeOH (0.8 mL) and H₂O (0.4 mL) was added NaOH (52.67 mg, 1.32 mmol, 5 eq) at 20° C. Then the mixture was stirred at 40° C. for 2 hours. The mixture was adjusted to pH=7 with sodium bicarbonate aqueous solution. Then reaction mixture was concentrated under reduced pressure to give 5-[4-[[(3R)-1-tert-butoxycarbonyl-3-piperidyl]-[2-fluoro-4-(5-methyl-1,3,4-thiadiazol-2-yl)benzoyl]amino]thieno[3,2-c]pyridin-2-yl]pent-4-ynoic acid (0.21 g, 78%) as a yellow oil. LC-MS: [M+H]=650.3.

### Step 6:

To a solution of 5-[4-[[(3R)-1-tert-butoxycarbonyl-3-piperidyl]-[2-fluoro-4-(5-methyl-1,3,4-thiadiazol-2-yl)benzoyl]amino]thieno[3,2-c]pyridin-2-yl]pent-4-ynoic acid (0.21 g, 206.52 μmol, 1 eq) ammonium chloride (55.24 mg, 1.03 mmol, 5 eq) in DMF (2 mL) was added HATU (157.05 mg, 413.05 μmol, 2 eq), DIEA (80.07 mg, 619.57 μmol, 107.92 μL, 3 eq) at 20° C. Then the mixture was stirred at 20° C. for 2 hours. The reaction mixture was diluted with water (10 mL) and extracted with EtOAc (3×10 mL). The combined organic layer was washed with brine (3×10 mL), dried over Na₂SO₄, filtered and concentrated under reduced pressure to give a residue. The residue was purified by reverse phase prep-HPLC to give tert-butyl (3R)-3-[[2-(5-amino-5-oxo-pent-1-ynyl)thieno[3,2-c]pyridin-4-yl]-[2-fluoro-4-(5-methyl-1,3,4-thiadiazol-2-yl)benzoyl]-amino]piperidine-1-carboxylate (0.04 g, 23%) as a yellow solid. LC-MS: [M+H]=649.3.

### Step 7:

The title compound hydrochloride salt was obtained as an off-white solid by deprotection Method A used in Example 97. LC-MS: [M+H]=549.1. ¹H NMR (400 MHZ, DMSO-d6): δ=8.74-9.16 (m, 2H), 8.28-8.41 (m, 1H), 7.97 (br d, J=5.25 Hz, 1H), 7.74 (br s, 1H), 7.54-7.66 (m, 1H), 7.37-7.52 (m, 2H), 7.21-7.35 (m, 1H), 6.89-7.01 (m, 1H), 4.78-5.04 (m, 1H), 3.09-3.32 (m, 2H), 2.73 (s, 8H), 2.36-2.44 (m, 2H), 1.97 (br d, J=10.13 Hz, 1H), 1.64-1.85 (m, 3H), 1.07-1.29 (m, 1H).

### Synthesis of (R)-2-fluoro-N-(2-(4-hydroxybut-1-yn-1-yl)thieno[3,2-c]pyridin-4-yl)-4-(5-methyl-1,3,4-thiadiazol-2-yl)-N-(piperidin-3-yl)benzamide (Example 818)

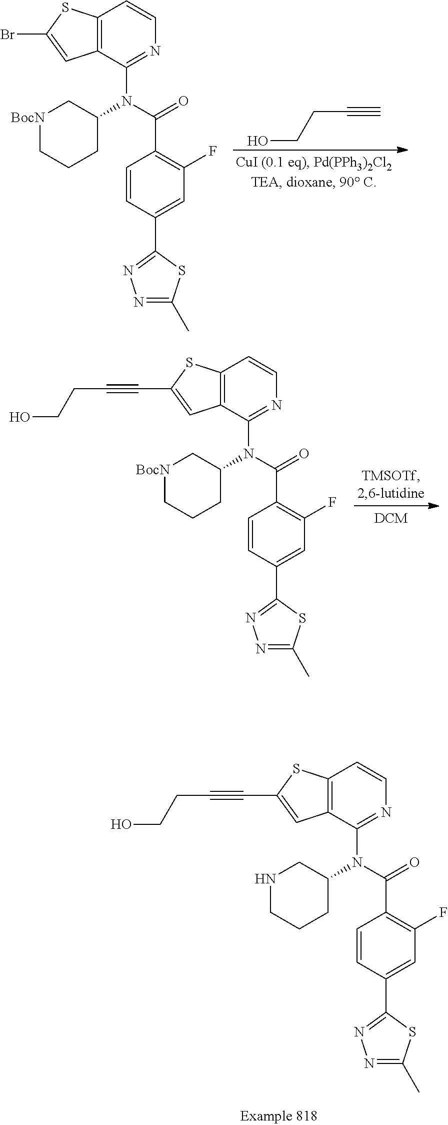

### Step 1:

To a solution of tert-butyl (R)-3-(N-(2-bromothieno[3,2-c]pyridin-4-yl)-2-fluoro-4-(5-methyl-1,3,4-thiadiazol-2-yl)benzamido)piperidine-1-carboxylate (140 mg, 221.32 μmol) in dioxane (2 mL) was added CuI (8.43 mg, 44.26 μmol), but-3-yn-1-ol (77.56 mg, 1.11 mmol), Pd(dppf)Cl₂ (16.19 mg, 22.13 μmol) and TEA (223.95 mg, 2.21 mmol) under N₂ at 25° C. Then the mixture was heated to 100° C. and stirred for 2 h. The reaction mixture was concentrated under reduced pressure to give a residue. The residue was purified by reverse phase prep-HPLC to afford tert-butyl (R)-3-(N-(2-bromothieno[3,2-c]pyridin-4-yl)-2-fluoro-4-(5-methyl-1,3,4-thiadiazol-2-yl)benzamido)piperidine-1-carboxylate (60 mg, 43%) as yellow solid.

### Step 2:

To a solution of tert-butyl (R)-3-(N-(2-bromothieno[3,2-c]pyridin-4-yl)-2-fluoro-4-(5-methyl-1,3,4-thiadiazol-2-yl)benzamido)piperidine-1-carboxylate (50 mg, 80.42 μmol) in DCM (2 mL) was added TMSOTf (53.62 mg, 241.26 μmol) and 2,6-dimethylpyridine (25.85 mg, 241.26 μmol). The mixture was stirred at 0° C. for 1 h. The reaction mixture was concentrated under reduced pressure to give a residue. The residue was purified by reverse phase prep-HPLC to afford tert-butyl (R)-3-(N-(2-bromothieno[3,2-c]pyridin-4-yl)-2-fluoro-4-(5-methyl-1,3,4-thiadiazol-2-yl)benzamido)piperidine-1-carboxylate (7 mg, 16%) as yellow solid. LC-MS: [M+H⁺]=522.1. ¹H NMR (400 MHZ, CD₃OD): δ=8.31 (br d, J=0.9 Hz, 1H), 7.75 (br d, J=4.1 Hz, 1H), 7.59-7.36 (m, 3H), 7.25 (br d, J=2.1 Hz, 1H), 4.80-4.59 (m, 1H), 3.77 (t, J=6.4 Hz, 2H), 3.50 (br d, J=12.1 Hz, 1H), 3.16-2.99 (m, 1H), 2.93 (br d, J=10.9 Hz, 1H), 2.81-2.68 (m, 5H), 2.59-2.24 (m, 2H), 2.07-1.91 (m, 1H), 1.88-1.60 (m, 2H), 1.32-1.10 (m, 1H).

### Synthesis of N-[2-(4-carbamoylphenyl)thieno[3,2-c] yridine-4-yl]-4-(1,5-dimethyltriazol-4-yl)-2-fluoro-N-[(3R)-3-piperidyl]benzamide (Example 819)

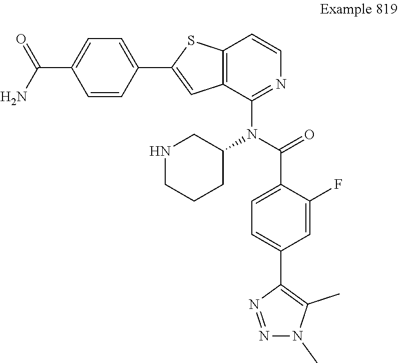 [SMILES: Cc1c(-c2ccc(C(=O)N(c3nccc4sc(-c5ccc(C(N)=O)cc5)cc34)[C@@H]3CCCNC3)c(F)c2)nnn1C]

Example 819 was prepared according to the procedure described for Example 161 by substituting 4-pyridylboronic acid and 2-fluoro-4-(triazolo[4,5-b]pyridin-3-yl)benzoyl chloride with (4-carbamoylphenyl)boronic acid and 4-(1,5-dimethyltriazol-4-yl)-2-fluoro-benzoyl chloride, provided the title compound hydrochloride salt as an off-white solid. LC-MS: [M+H⁺]=570.3. ¹H NMR (400 MHZ, DMSO-d6): δ=9.43-8.92 (m, 2H), 8.37 (br d, J=5.4 Hz, 1H), 8.13 (br d, J=6.3 Hz, 2H), 8.05-7.88 (m, 5H), 7.50 (br s, 1H), 7.33-7.05 (m, 3H), 5.24-4.87 (m, 1H), 3.88 (br s, 3H), 3.70 (br s, 1H), 3.41-3.11 (m, 2H), 2.76-2.62 (m, 1H), 2.26 (s, 3H), 2.13-1.71 (m, 3H), 1.26-1.09 (m, 1H).

### Synthesis of (R)-4-(3H-[1,2,3]triazolo[4,5-b]pyridin-3-yl)-N-(2,3-dimethylthieno[3,2-c]pyridin-4-yl)-2-fluoro-N-(piperidin-3-yl)benzamide (Example 820)

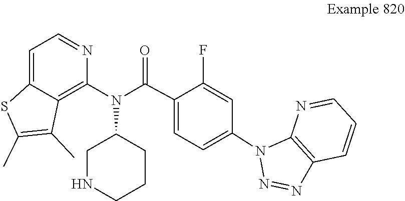 [SMILES: Cc1sc2ccnc(N(C(=O)c3ccc(-n4nnc5cccnc54)cc3F)[C@@H]3CCCNC3)c2c1C]

Example 820 was prepared according to the procedure described for Example 241 by substituting tert-butyl (R)-3-((8-methylisoquinolin-1-yl)amino)piperidine-1-carboxylate with tert-butyl (R)-3-((2,3-dimethylthieno[3,2-c]pyridin-4-yl)amino)piperidine-1-carboxylate and ethyl 4-aminobenzoate with methyl 4-amino-2-fluorobenzoate; provided the title compound as off white solid. LC-MS: [M+H⁺]=502.20. ¹H NMR (400 MHZ, DMSO-d₆): δ=9.04-8.89 (m, 2H), 8.83 (dd, J=1.4, 4.5 Hz, 1H), 8.69 (dd, J=1.4, 8.4 Hz, 1H), 8.36-8.28 (m, 1H), 8.09-8.02 (m, 1H), 7.98-7.94 (m, 1H), 7.92-7.85 (m, 1H), 7.62 (dd, J=4.6, 8.4 Hz, 1H), 7.36-7.22 (m, 1H), 5.01-4.88 (m, 1H), 3.79 (br d, J=11.9 Hz, 1H), 3.29-3.18 (m, 2H), 2.84-2.70 (m, 1H), 2.45 (d, J=8.4 Hz, 6H), 1.86-1.65 (m, 3H), 1.09-0.92 (m, 1H).

### Synthesis of (R)-4-(1-ethyl-1H-1,2,3-triazol-4-yl)-2-fluoro-N-(8-methylisoquinolin-1-yl)-N-(piperidin-3-yl)benzamide (Example 821)

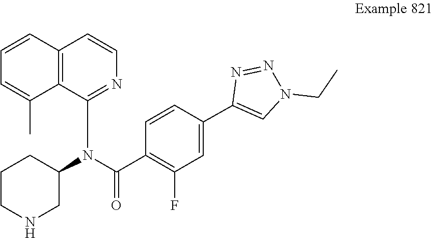 [SMILES: CCn1cc(-c2ccc(C(=O)N(c3nccc4cccc(C)c34)[C@@H]3CCCNC3)c(F)c2)nn1]

Example 821 was synthesized following the procedure for example 127 by substituting 2-bromo-5-methyl-1,3,4-thiadiazole with 4-bromo-1-ethyl-1H-1,2,3-triazole to afford (R)-4-(1-ethyl-1H-1,2,3-triazol-4-yl)-2-fluoro-N-(8-methylisoquinolin-1-yl)-N-(piperidin-3-yl) benzamide hydrochloride salt (20 mg, 97%) as an off-white solid. LC-MS: [M+H⁺]=459.1. ¹H NMR (CD₃OD, 400 MHZ): δ 8.45-8.39 (m, 1H), 8.22 (s, 1H), 7.27-7.68 (m, 1H), 7.64-7.59 (m, 1H), 7.51-7.45 (m, 2H), 7.26-7.19 (m, 1H), 7.12-7.11 (m, 1H), 6.87-6.82 (m, 1H), 5.09-5.03 (m, 1H), 4.39 (q, J=7.6 Hz, 2H), 4.00-3.98 (m, 1H), 3.85-3.82 (m, 1H), 3.41-3.34 (m, 1H), 3.03-3.00 (m, 1H), 2.83 (s, 3H), 2.72-2.69 (m, 1H), 1.98-1.91 (m, 2H), 1.62-1.55 (m, 1H), 1.49 (t, J=7.6 Hz, 3H).

### Synthesis of (R)-2-chloro-4-(1-methyl-1H-1,2,3-triazol-4-yl)-N-(8-methylisoquinolin-1-yl)-N-(piperidin-3-yl)benzamide (Example 822)

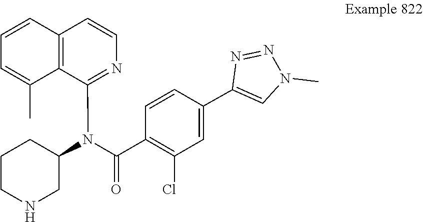 [SMILES: Cc1cccc2ccnc(N(C(=O)c3ccc(-c4cn(C)nn4)cc3Cl)[C@@H]3CCCNC3)c12]

Example 822 was prepared according to the procedure described for Example 127 by substituting 2-fluoro-4-iodobenzoyl chloride and 2-bromo-5-methyl-1,3,4-thiadiazole with 4-bromo-2-chloro-benzoyl chloride and 4-bromo-1-methyl-triazole, provided the title compound hydrochloride salt as a yellow solid. LC-MS: [M+H⁺]=461.4. ¹H NMR (400 MHz, DMSO-d6): δ=9.74 (br d, J=10.3 Hz, 1H), 9.38 (br d, J=10.4 Hz, 1H), 8.45 (s, 1H), 8.37 (d, J=5.4 Hz, 1H), 7.77-7.68 (m, 2H), 7.62-7.51 (m, 3H), 7.24 (d, J=8.1 Hz, 1H), 6.87 (d, J=8.1 Hz, 1H), 4.99-4.87 (m, 1H), 3.99 (s, 3H), 3.86 (br d, J=11.1 Hz, 1H), 3.50 (q, J=10.9 Hz, 1H), 3.22 (br d, J=11.9 Hz, 1H), 3.00 (s, 1H), 2.86 (s, 3H), 2.81-2.69 (m, 1H), 1.97-1.72 (m, 3H), 1.37-1.19 (m, 1H).

### Synthesis of (R)-2-fluoro-N-(8-methylisoquinolin-1-yl)-N-(piperidin-3-yl)-4-(thieno[2,3-d]pyrimidin-2-ylamino)benzamide (Example 823)

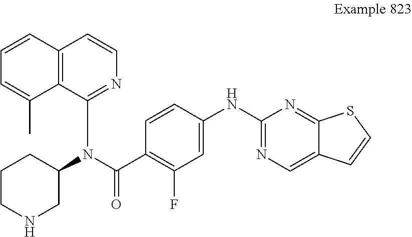 [SMILES: Cc1cccc2ccnc(N(C(=O)c3ccc(Nc4ncc5ccsc5n4)cc3F)[C@@H]3CCCNC3)c12]

Example 823 was synthesized following the procedure for example 210 by substituting 2-chloropyrimidine with 2-chlorothieno[2,3-d]pyrimidine to afford the title compound as a pale yellow solid. LC-MS: [M+H⁺]=513.2. ¹H NMR (CD₃OD, 400 MHZ): δ 8.86 (s, 1H), 8.45 (d, J=5.6 Hz, 1H), 7.75 (d, J=5.6 Hz, 1H), 7.67-7.63 (m, 1H), 7.58-7.54 (m, 1H), 7.51-7.42 (m, 2H), 7.37 (d, J=6.0 Hz, 1H), 7.27 (d, J=6.0 Hz, 1H), 6.90 (dd, J=8.8 Hz, 2.0 Hz, 1H), 6.65 (t, J=8.0 Hz, 1H), 5.10-5.02 (m, 1H), 3.98-3.94 (m, 1H), 3.85-3.79 (m, 1H), 3.41-3.38 (m, 1H), 3.02-3.00 (m, 1H), 2.81 (s, 3H), 1.98-1.88 (m, 3H), 1.60-1.56 (m, 1H).

### Synthesis of (R)-2-fluoro-N-(8-methylisoquinolin-1-yl)-N-(piperidin-3-yl)-4-(quinazolin-2-ylamino)benzamide (Example 824)

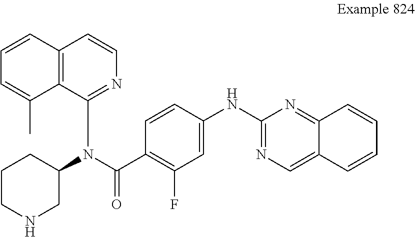 [SMILES: Cc1cccc2ccnc(N(C(=O)c3ccc(Nc4ncc5ccccc5n4)cc3F)[C@@H]3CCCNC3)c12]

Example 824 was synthesized following the procedure for example 131 by substituting 3-amino-1-methylpyridin-2 (1H)-one with quinazolin-2-amine to afford the title compound as a yellow solid. LC-MS: [M+H⁺]=507.1. 1H NMR (CD₃OD, 400 MHZ): δ 8.46-8.41 (m, 1H), 7.78-7.70 (m, 3H), 7.55-7.41 (m, 4H), 7.38-7.29 (m, 1H), 7.16-7.12 (m, 1H), 7.03-7.02 (m, 1H), 6.94-6.66 (m, 1H), 6.75-6.72 (m, 1H), 5.10-5.07 (m, 1H), 4.01-3.98 (m, 1H), 3.82-3.76 (m, 1H), 3.44-3.38 (m, 1H), 3.04-2.98 (m, 1H), 2.84 (s, 3H), 2.72-2.68 (m, 1H), 1.94-1.92 (m, 2H), 1.60-1.40 (m, 1H).

### Synthesis of N-[2-(4-carbamoylphenyl)thieno[3,2-c]pyridin-4-yl]-2-fluoro-N-[(3R)-3-piperidyl]-4-(4,5,6,7-tetrahydropyrazolo[1,5-a]pyridin-3-yl)benzamide (Example 825)

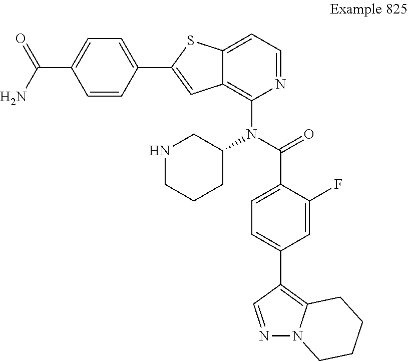 [SMILES: NC(=O)c1ccc(-c2cc3c(N(C(=O)c4ccc(-c5cnn6c5CCCC6)cc4F)[C@@H]4CCCNC4)nccc3s2)cc1]

Example 825 was prepared according to the procedure described for Example 819 by substituting 4-bromo-1,5-dimethyl-triazole with 3-Bromo-4,5,6,7-tetrahydropyrazolo[1,5-a]pyridine, provided the title compound as a white solid. LC-MS: [M+H⁺]=595.3. ¹H NMR (400 MHZ, CD₃OD): δ=8.37 (d, J=5.5 Hz, 1H), 8.01 (d, J=8.3 Hz, 2H), 7.93-7.80 (m, 5H), 7.17 (br t, J=7.4 Hz, 1H), 7.06-6.93 (m, 2H), 5.31-5.18 (m, 1H), 4.15 (br t, J=5.7 Hz, 2H), 3.89 (br d, J=10.9 Hz, 1H), 3.75-3.64 (m, 1H), 3.37 (br s, 2H), 2.95-2.83 (m, 1H), 2.82-2.66 (m, 2H), 2.04 (br d, J=3.9 Hz, 3H), 1.99-1.76 (m, 4H), 1.48-1.35 (m, 1H).

### Synthesis of 4-(benzimidazol-1-yl)-N-[2-(4-carbamoylphenyl)thieno[3,2-c]pyridin-4-yl]-2-fluoro-N-[(3R)-3-piperidyl]benzamide (Example 826)

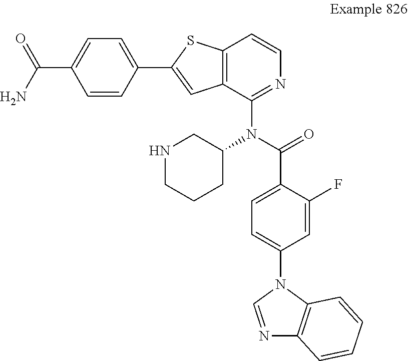 [SMILES: NC(=O)c1ccc(-c2cc3c(N(C(=O)c4ccc(-n5cnc6ccccc65)cc4F)[C@@H]4CCCNC4)nccc3s2)cc1]

Example 826 was prepared according to the procedure described for Example 161 by substituting 4-pyridylboronic acid and 2-fluoro-4-(triazolo[4,5-b]pyridin-3-yl)benzoyl chloride with (4-carbamoylphenyl)boronic acid and 4-(benzimidazol-1-yl)-2-fluoro-benzoyl chloride, provided the title compound hydrochloride salt as an off-white solid. LC-MS: [M+H⁺]=591.3. ¹H NMR (400 MHZ, DMSO-d₆): δ=9.18-8.89 (m, 2H), 8.63-8.58 (m, 1H), 8.40 (br d, J=5.4 Hz, 1H), 8.25-8.20 (m, 1H), 8.14-7.96 (m, 7H), 7.73 (br d, J=7.8 Hz, 1H), 7.55-7.45 (m, 3H), 7.33-7.25 (m, 4H), 5.19-4.92 (m, 1H), 3.43-3.34 (m, 1H), 3.26-3.16 (m, 1H), 2.78-2.70 (m, 1H), 2.15-2.05 (m, 1H), 1.94-1.68 (m, 3H), 1.26-1.17 (m, 1H).

### Synthesis of N-[2-(4-carbamoylphenyl)thieno[3,2-c]pyridin-4-yl]-2-fluoro-4-(5-methyl-1,3,4-thiadiazol-2-yl)-N-[(3R)-3-piperidyl]benzamide (Example 827)

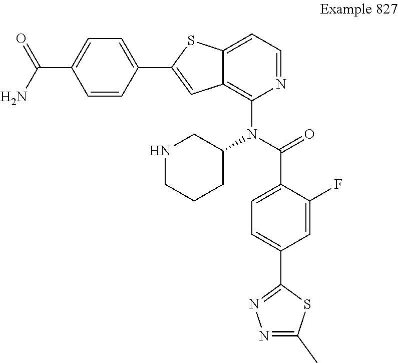 [SMILES: Cc1nnc(-c2ccc(C(=O)N(c3nccc4sc(-c5ccc(C(N)=O)cc5)cc34)[C@@H]3CCCNC3)c(F)c2)s1]

Example 827 was prepared according to the procedure described for Example 161 by substituting 4-pyridylboronic acid and 2-fluoro-4-(triazolo[4,5-b]pyridin-3-yl)benzoyl chloride with (4-carbamoylphenyl) boronic acid and 2-fluoro-4-(5-methyl-1,3,4-thiadiazol-2-yl)benzoyl chloride, provided the title compound hydrochloride salt as an off-white solid. LC-MS: [M+H⁺]=573.2. ¹H NMR (400 MHZ, DMSO-d₆): δ=9.44-8.96 (m, 2H), 8.40-8.31 (m, 1H), 8.25-8.18 (m, 1H), 8.12 (br s, 1H), 8.05-7.94 (m, 5H), 7.59-7.47 (m, 2H), 7.47-7.31 (m, 2H), 5.15-4.94 (m, 1H), 3.72 (br d, J=10.4 Hz, 1H), 3.38-3.27 (m, 1H), 3.18 (br d, J=11.1 Hz, 1H), 2.70 (s, 3H), 2.26-2.04 (m, 1H), 1.94-1.67 (m, 3H), 1.25-1.07 (m, 1H).

### Synthesis of (R)-2-fluoro-4-(1-methyl-1H-1,2,3-triazol-4-yl)-N-(piperidin-3-yl)-N-(6-(pyridin-3-ylmethyl)isoquinolin-1-yl)benzamide (Example 828)

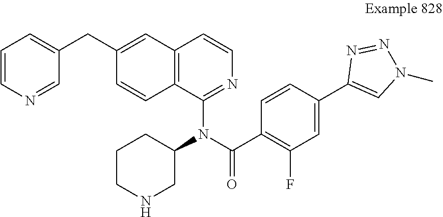 [SMILES: Cn1cc(-c2ccc(C(=O)N(c3nccc4cc(Cc5cccnc5)ccc34)[C@@H]3CCCNC3)c(F)c2)nn1]

Example 828 was prepared according to the procedure described for Example 574 by substituting 2-bromo-5-methylthiazole with 3-(bromomethyl)pyridine hydrobromide and tert-butyl (R)-3-(4-(3H-[1,2,3]triazolo[4,5-b]pyridin-3-yl)-2-fluoro-N-(6-(4,4,5,5-tetramethyl-1,3,2-dioxaborolan-2-yl) isoquinolin-1-yl) benzamido) piperidine-1-carboxylate with tert-butyl (R)-3-(2-fluoro-4-(1-methyl-1H-1,2,3-triazol-4-yl)-N-(6-(4,4,5,5-tetramethyl-1,3,2-dioxaborolan-2-yl)isoquinolin-1-yl)benzamido)piperidine-1-carboxylate to afford the title compound as an off-white solid. LC-MS: [M+H⁺]=522.3. ¹H NMR (CD₃OD, 400 MHZ): δ 8.81-8.75 (m, 2H), 8.45-8.41 (m, 2H), 8.18 (s, 1H), 8.14-8.10 (m, 1H), 8.03-8.00 (m, 1H), 7.80-7.71 (m, 2H), 7.63 (d, J=8.4 Hz, 1H), 7.24-7.21 (m, 1H), 7.15-7.13 (m, 1H), 7.05-7.01 (m, 1H), 5.25-5.22 (m, 1H), 4.4 (s, 2H), 4.08 (s, 3H), 3.93-3.90 (m, 1H), 3.72-3.65 (m, 1H), 3.35-3.34 (m, 1H), 2.86-2.83 (m, 1H), 2.03-1.88 (m, 3H), 1.28-1.24 (m, 1H).

### Synthesis of (R)-4-(1,5-dimethyl-1H-1,2,3-triazol-4-yl)-2-fluoro-N-(6-fluoro-8-methylisoquinolin-1-yl)-N-(piperidin-3-yl)benzamide (Example 829)

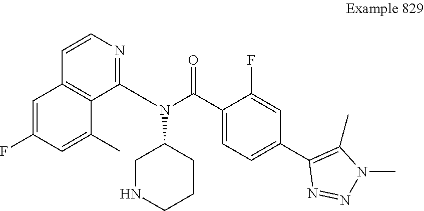 [SMILES: Cc1cc(F)cc2ccnc(N(C(=O)c3ccc(-c4nnn(C)c4C)cc3F)[C@@H]3CCCNC3)c12]

Example 829 was synthesized following the procedure for Example 833 by 2-bromo-5-methyl-1,3,4-thiadiazole with 4-bromo-1,5-dimethyl-1H-1,2,3-triazole to afford the title compound as an off-white solid. LC-MS: [M+H⁺]=477.2. ¹H NMR (CD₃OD, 400 MHZ): δ 8.43-8.35 (m, 1H), 7.71-7.67 (m, 1H), 7.35-7.28 (m, 2H), 7.15-7.10 (m, 1H), 7.04-7.02 (m, 1H), 6.96-6.92 (m, 1H), 5.11-5.04 (m, 1H), 4.00 (s, 3H), 3.98-3.95 (m, 1H), 3.86-3.80 (m, 1H), 3.45-3.36 (m, 1H), 3.05-3.01 (m, 1H), 2.85 (s, 3H), 2.72-2.70 (m, 1H), 2.35 (s, 3H), 1.98-1.92 (m, 2H), 1.66-1.59 (m, 1H).

### Synthesis of (R)-4-(1,5-dimethyl-1H-1,2,3-triazol-4-yl)-2-fluoro-N-(8-methylisoquinolin-1-yl)-N-(piperidin-3-yl)benzamide (Example 830)

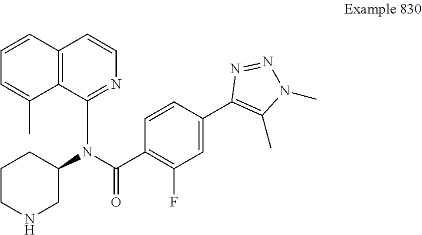 [SMILES: Cc1cccc2ccnc(N(C(=O)c3ccc(-c4nnn(C)c4C)cc3F)[C@@H]3CCCNC3)c12]

Example 830 was synthesized following the procedure for example 127 by substituting 2-bromo-5-methyl-1,3,4-thiadiazole with 4-bromo-1,5-dimethyl-1H-1,2,3-triazole to afford (R)-4-(1,5-dimethyl-1H-1,2,3-triazol-4-yl)-2-fluoro-N-(8-methylisoquinolin-1-yl)-N-(piperidin-3-yl) benzamide hydrochloride salt (23 mg, 99%) as an off-white solid. LC-MS: [M+H⁺]=459.2. ¹H NMR (CD₃OD, 400 MHZ): δ 8.43 (d, J=5.6 Hz, 1H), 7.74 (d, J=5.6 Hz, 1H), 7.66-7.61 (m, 1H), 7.53-7.45 (m, 2H), 7.13-7.06 (m, 1H), 7.01-6.93 (m, 2H), 5.11-5.04 (m, 1H), 4.05-3.98 (m, 1H), 4.01 (s, 3H), 3.45-3.39 (m, 1H), 3.04-2.98 (m, 1H), 2.84 (s, 3H), 2.74-2.70 (m, 1H), 2.31 (s, 3H), 1.98-1.92 (m, 2H), 1.62-1.58 (m, 1H).

### Synthesis of (R)—N-(2,3-dimethylthieno[3,2-c]pyridin-4-yl)-2-fluoro-4-(1-methyl-1H-1,2,3-triazol-4-yl)-N-(piperidin-3-yl)benzamide (Example 831)

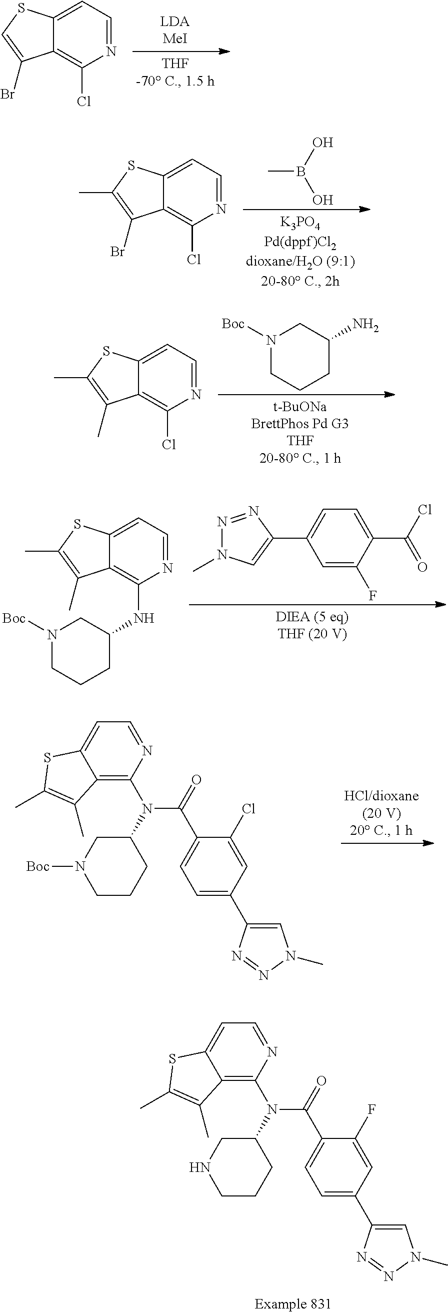

### Step 1:

To a solution of 3-bromo-4-chloro-thieno[3,2-c]pyridine (1 g, 4.02 mmol) in THF (30 mL) was added LDA (2 M, 3.02 mL) at −70° C. under N₂ atmosphere. The mixture was stirred at −70° C. for 0.5 h. Then Mel (1.71 g, 12.07 mmol, 751.47 μL) was dropwise added into the mixture at −70° C. under N₂ atmosphere. The mixture was stirred at −70° C. for 1 h before it was poured into NH₄Cl aqueous solution (100 mL) and extracted with EtOAc (3×100 mL). The combined organic phase was washed with brine (200 mL), dried with anhydrous Na₂SO₄, concentrated in vacuum to give a residue. The residue was purified by column chromatography (SiO₂, Petroleum ether/EtOAc=95/5 to 85/15) to give 3-bromo-4-chloro-2-methyl-thieno[3,2-c]pyridine (1 g, 81%) as an off white solid. LC-MS: [M+H⁺]=261.8.

### Step 2:

To a solution of 3-bromo-4-chloro-2-methyl-thieno[3,2-c]pyridine (1.3 g, 4.70 mmol) and methylboronic acid (844.71 mg, 14.11 mmol) in dioxane (25 mL) and H₂O (2.5 mL) was added K₃PO₄ (2.00 g, 9.41 mmol) and Pd(dppf)Cl₂ (103.25 mg, 141.11 μmol) in order at 20° C. under N₂ atmosphere. Then the mixture was stirred at 80° C. for 2 h before the reaction mixture was poured into water (50 mL) and extracted with EtOAc (3×50 mL). The combined organic phase was washed with brine (100 mL), dried with anhydrous Na₂SO₄, concentrated in vacuum to give residue. The residue was purified by column chromatography (SiO₂, Petroleum/EtOAc=95/5 to 90/10) and the fraction was concentrated in vacuum to give 4-chloro-2,3-dimethyl-thieno[3,2-c]pyridine (0.65 g, 62%) as a yellow solid. LC-MS: [M+H⁺]=198.0.

### Steps 3-5:

Example 831 was prepared according to the procedure described for Example 444 by substituting 1,6-dichloro-8-methylisoquinoline with 4-chloro-2,3-dimethyl-thieno[3,2-c]pyridine, provided the title compound hydrochloride salt as a brown solid. LC-MS: [M+H⁺]=465.2. ¹H NMR (400 MHZ, DMSO-d₆): δ ppm 9.53-9.25 (m, 1H), 9.20-8.95 (m, 1H), 8.48 (s, 1H), 8.36-8.24 (m, 1H), 7.97-7.84 (m, 1H), 7.48-7.36 (m, 1H), 7.33-7.21 (m, 1H), 7.13-6.92 (m, 1H), 5.05-4.66 (m, 1H), 4.02 (s, 3H), 3.75-3.64 (m, 1H), 3.49-3.06 (m, 2H), 2.81-2.66 (m, 1H), 2.48-2.37 (m, 6H), 2.03-1.58 (m, 3H), 1.12-0.80 (m, 1H).

### Synthesis of (R)-4-(5-ethyl-1,3,4-thiadiazol-2-yl)-2-fluoro-N-(6-fluoro-8-methylisoquinolin-1-yl)-N-(piperidin-3-yl)benzamide (Example 832)

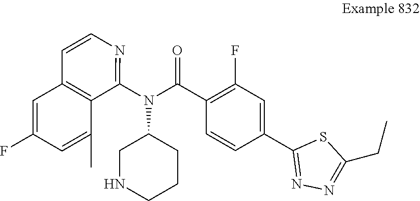 [SMILES: CCc1nnc(-c2ccc(C(=O)N(c3nccc4cc(F)cc(C)c34)[C@@H]3CCCNC3)c(F)c2)s1]

Example 832 was synthesized following the procedure for 833 by 2-bromo-5-methyl-1,3,4-thiadiazole with 2-bromo-5-ethyl-1,3,4-thiadiazole to afford the title compound as an off-white solid. LC-MS: [M+H⁺]=494.20. ¹H NMR (CD₃OD, 400 MHZ): δ 8.43-8.39 (m, 1H), 7.72-7.71 (m, 1H), 7.33-7.31 (m, 4H), 7.01-6.96 (m, 1H), 5.09-5.01 (m, 1H), 4.01-3.98 (m, 1H), 3.84-3.78 (m, 1H), 3.44-3.39 (m, 1H), 3.11 (q, J=7.6 Hz, 2H), 3.05-3.00 (m, 1H), 2.82 (s, 3H), 2.72-2.69 (m, 1H), 1.98-1.92 (m, 2H), 1.61-1.57 (m, 1H), 1.38 (t, J=7.6 Hz, 3H).

### Synthesis of (R)-2-fluoro-N-(6-fluoro-8-methylisoquinolin-1-yl)-4-(5-methyl-1,3,4-thiadiazol-2-yl)-N-(piperidin-3-yl)benzamide (Example 833)

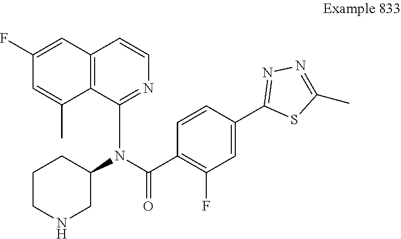 [SMILES: Cc1nnc(-c2ccc(C(=O)N(c3nccc4cc(F)cc(C)c34)[C@@H]3CCCNC3)c(F)c2)s1]

Example 833 was prepared according to the procedure described for Example 127 by substituting tert-butyl (R)-3-((8-methylisoquinolin-1-yl)amino)piperidine-1-carboxylate with tert-butyl (R)-3-((6-fluoro-8-methylisoquinolin-1-yl)amino)piperidine-1-carboxylate, provided the title compound hydrochloride salt as an off-white solid. LC-MS: [M+H⁺]=480.2. ¹H NMR (CD₃OD, 400 MHZ): δ 8.43-8.39 (m, 1H), 7.72-7.71 (m, 1H), 7.48-7.38 (m, 1H), 7.33-7.31 (m, 3H), 7.01-6.96 (m, 1H), 5.08-5.01 (m, 1H), 4.01-3.98 (m, 1H), 3.84-3.78 (m, 1H), 3.44-3.28 (m, 1H), 3.04-2.99 (m, 1H), 2.85 (s, 3H), 2.71-2.62 (m, 1H), 1.97-1.88 (m, 2H), 1.60-1.56 (m, 1H).

### Synthesis of (R)—N-(6-(4-(aminomethyl)phenyl)isoquinolin-1-yl)-2-fluoro-4-(1-methyl-1H-1,2,3-triazol-4-yl)-N-(piperidin-3-yl)benzamide (Example 834)

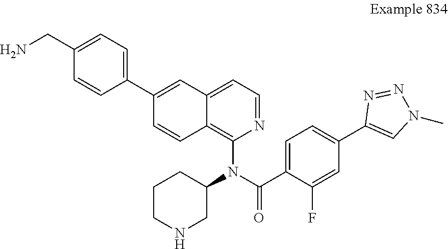 [SMILES: Cn1cc(-c2ccc(C(=O)N(c3nccc4cc(-c5ccc(CN)cc5)ccc34)[C@@H]3CCCNC3)c(F)c2)nn1]

Example 834 was prepared according to the procedure for Example 370 by substituting pyridin-3-ylboronic acid with (4-(4,4,5,5-tetramethyl-1,3,2-dioxaborolan-2-yl)phenyl)methanamine to afford the title compound (60 mg, 74%) as an off-white solid. LC-MS: [M+H⁺]=536.3. ¹H NMR (CD₃OD, 400 MHZ): δ 8.47-8.45 (m, 1H), 8.25-8.23 (m, 1H), 8.16-8.13 (m, 2H), 8.05-8.00 (m, 1H), 7.85-7.83 (m, 3H), 7.61-7.51 (m, 2H), 7.29-7.26 (m, 1H), 7.22-7.20 (m, 1H), 6.76-6.74 (m, 1H), 5.31-5.27 (m, 1H), 5.18 (s, 2H), 5.04 (s, 3H), 3.96-3.93 (m, 1H), 3.72-3.66 (m, 1H), 3.36-3.30 (m, 1H), 2.89-2.87 (m, 1H), 2.04-1.90 (m, 3H), 1.39-1.19 (m, 1H).

### Synthesis of (R)-4-(5-cyclopropyl-1,3,4-thiadiazol-2-yl)-2-fluoro-N-(8-methylisoquinolin-1-yl)-N-(piperidin-3-yl)benzamide (Example 836)

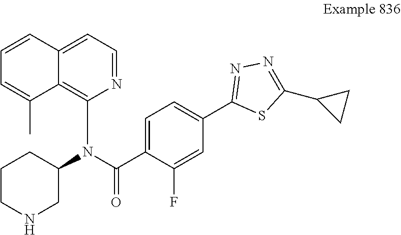 [SMILES: Cc1cccc2ccnc(N(C(=O)c3ccc(-c4nnc(C5CC5)s4)cc3F)[C@@H]3CCCNC3)c12]

Example 836 was synthesized following the procedure for example 127 by substituting 2-bromo-5-methyl-1,3,4-thiadiazole with 2-bromo-5-cyclopropyl-1,3,4-thiadiazole to afford (R)-4-(5-cyclopropyl-1,3,4-thiadiazol-2-yl)-2-fluoro-N-(8-methylisoquinolin-1-yl)-N-(piperidin-3-yl) benzamide hydrochloride salt (48 mg, 83%) as an off-white solid. LC-MS: [M+H⁺]=488.0. ¹H NMR (CD₃OD, 400 MHz): δ 8.43-8.40 (m, 1H), 7.75-7.70 (m, 1H), 7.66-7.62 (m, 1H), 7.54-7.48 (m, 2H), 7.39-7.32 (m, 1H), 7.26-7.21 (m, 1H), 6.99-6.94 (m, 1H), 5.09-5.03 (m, 1H), 4.02-3.99 (m, 1H), 3.84-3.81 (m, 1H), 3.42-3.39 (m, 1H), 3.03-2.98 (m, 1H), 2.84 (s, 3H), 2.73-2.71 (m, 1H), 2.45-2.43 (m, 1H), 1.95-1.91 (m, 2H), 1.59-1.53 (m, 1H), 1.29-1.27 (m, 2H), 1.09-1.08 (m, 2H).

### Synthesis of (R)-4-(5-ethyl-1,3,4-thiadiazol-2-yl)-2-fluoro-N-(8-methylisoquinolin-1-yl)-N-(piperidin-3-yl)benzamide (Example 837)

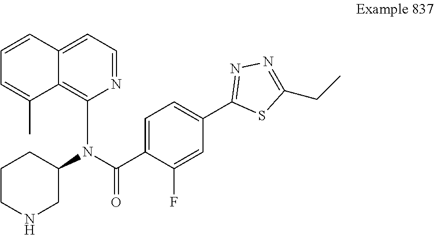 [SMILES: CCc1nnc(-c2ccc(C(=O)N(c3nccc4cccc(C)c34)[C@@H]3CCCNC3)c(F)c2)s1]

Example 837 was synthesized following the procedure for example 127 by substituting 2-bromo-5-methyl-1,3,4-thiadiazole with 2-bromo-5-ethyl-1,3,4-thiadiazole to afford (R)-4-(5-ethyl-1,3,4-thiadiazol-2-yl)-2-fluoro-N-(8-methylisoquinolin-1-yl)-N-(piperidin-3-yl) benzamide hydrochloride salt (38 mg, 92%) as an off-white solid. LC-MS: [M+H⁺]=476.2. ¹H NMR (CD₃OD, 400 MHZ): δ 8.43 (d, J=5.6 Hz, 1H), 7.74 (d, J=5.6 Hz, 1H), 7.67-7.62 (m, 1H), 7.54-7.48 (m, 2H), 7.43-7.39 (m, 1H), 7.27-7.25 (m, 1H), 7.00-6.95 (m, 1H), 5.10-5.02 (m, 1H), 4.03-4.00 (m, 1H), 3.85-3.79 (m, 1H), 3.42-3.38 (m, 1H), 3.10 (q, J=7.6 Hz, 2H), 3.05-2.98 (m, 1H), 2.82 (s, 3H), 1.97-1.92 (m, 2H), 1.60-1.55 (m, 1H), 1.38 (t, J=7.6 Hz, 3H), 1.28-1.20 (m, 1H).

### Synthesis of 2-fluoro-N-[2-(5-hydroxypent-1-ynyl)thieno[3,2-c]pyridin-4-yl]-N-[(3R)-3-piperidyl]-4-(4,5,6,7-tetrahydropyrazolo[1,5-a]pyridin-3-yl)benzamide (Example 839)

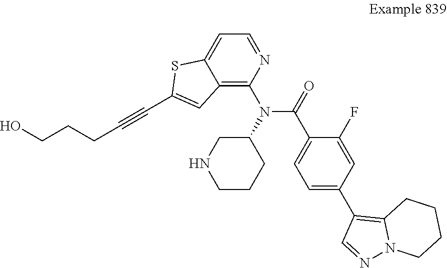 [SMILES: O=C(c1ccc(-c2cnn3c2CCCC3)cc1F)N(c1nccc2sc(C#CCCCO)cc12)[C@@H]1CCCNC1]

Example 839 was prepared according to the procedure described for Example 818 by substituting tert-butyl (R)-3-(N-(2-bromothieno[3,2-c]pyridin-4-yl)-2-fluoro-4-(5-methyl-1,3,4-thiadiazol-2-yl)benzamido)piperidine-1-carboxylate with tert-butyl (3R)-3-[(2-bromothieno[3,2-c]pyridin-4-yl)-[2-fluoro-4-(4,5,6,7-tetrahydropyrazolo[1,5-a]pyridin-3-yl)benzoyl]amino]-piperidine-1-carboxylate and but-3-yn-1-ol with pent-4-yn-1-ol, provided the title compound as a yellow solid. LC-MS: [M+H⁺]=558.4. ¹H NMR (400 MHZ, CD3OD): δ=8.35 (d, J=5.6 Hz, 1H), 7.79 (br d, J=5.6 Hz, 1H), 7.66 (s, 1H), 7.43 (s, 1H), 7.18-6.83 (m, 3H), 5.21-5.08 (m, 1H), 4.82 (br d, J=1.5 Hz, 2H), 4.12 (br t, J=5.8 Hz, 2H), 3.89-3.78 (m, 1H), 3.70 (t, J=6.2 Hz, 2H), 3.66-3.54 (m, 1H), 2.93-2.77 (m, 3H), 2.61 (t, J=7.2 Hz, 2H), 2.10-1.99 (m, 3H), 1.99-1.79 (m, 6H), 1.44-1.26 (m, 1H).

### Synthesis of N-[2-(4-carbamoylphenyl)thieno[3,2-c]pyridin-4-yl]-2-fluoro-4-(1-methyltriazol-4-yl)-N-[(3R)-3-piperidyl] benzamide (Example 840)

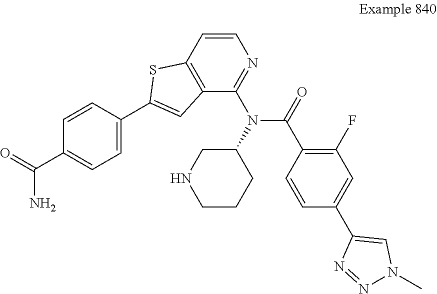 [SMILES: Cn1cc(-c2ccc(C(=O)N(c3nccc4sc(-c5ccc(C(N)=O)cc5)cc34)[C@@H]3CCCNC3)c(F)c2)nn1]

Example 840 was prepared according to the procedure described for Example 161 by substituting 2-fluoro-4-(triazolo[4,5-b]pyridin-3-yl)benzoyl chloride with 2-fluoro-4-(1-methyltriazol-4-yl)benzoyl chloride and 4-pyridylboronic acid with 4-(4,4,5,5-tetramethyl-1,3,2-dioxaborolan-2-yl)benzamide, provided the title compound as a white solid. LC-MS: [M+H⁺]=556.2. ¹H NMR (400 MHZ, CD₃OD): δ=8.37 (d, J=5.4 Hz, 1H), 8.17 (s, 1H), 8.00 (br d, J=8.3 Hz, 2H), 7.95-7.80 (m, 4H), 7.42-7.12 (m, 3H), 5.25 (br s, 1H), 4.06 (s, 3H), 3.96-3.79 (m, 1H), 3.76-3.62 (m, 1H), 3.35 (br d, J=13.1 Hz, 1H), 2.95-2.81 (m, 1H), 2.53-2.03 (m, 2H), 2.01-1.79 (m, 2H), 1.52-1.31 (m, 1H).

### Synthesis of (R)-2-fluoro-4-(1-methyl-1H-1,2,3-triazol-4-yl)-N-(3-methylthieno[3,2-c]pyridin-4-yl)-N-(piperidin-3-yl)benzamide (Example 841)

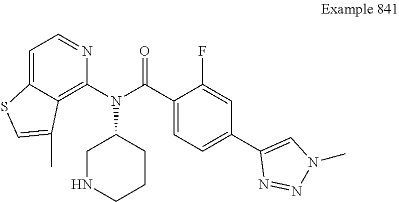 [SMILES: Cc1csc2ccnc(N(C(=O)c3ccc(-c4cn(C)nn4)cc3F)[C@@H]3CCCNC3)c12]

Example 841 was prepared according to the procedure described for Example 444 by substituting tert-butyl (3R)-3-[(6-chloro-1-isoquinolyl)amino]piperidine-1-carboxylate with tert-butyl (R)-3-((3-methylthieno[3,2-c]pyridin-4-yl)amino)piperidine-1-carboxylate, provided the title compound hydrochloride salt as a white solid. LC-MS: [M+H⁺]=451.2. ¹H NMR: (400 MHZ, CD₃OD): δ ppm 8.41-8.33 (m, 1H), 8.23 (s, 1H), 8.01-7.92 (m, 1H), 7.47 (br s, 1H), 7.36 (br d, J=11.3 Hz, 1H), 7.30-7.20 (m, 1H), 7.08 (br d, J=1.9 Hz, 1H), 5.14-5.02 (m, 1H), 4.08 (s, 3H), 3.98 (br d, J=11.8 Hz, 1H), 3.73 (br t, J=11.5 Hz, 1H), 3.38 (br d, J=12.3 Hz, 1H), 3.04-2.87 (m, 1H), 2.72-2.54 (m, 4H), 1.99-1.78 (m, 3H), 1.39-1.25 (m, 1H).

### Synthesis of (R)-2-fluoro-4-(1-methyl-1H-1,2,3-triazol-4-yl)-N-(piperidin-3-yl)-N-(3-(prop-1-en-2-yl)thieno[3,2-c]pyridin-4-yl)benzamide (Example 842)

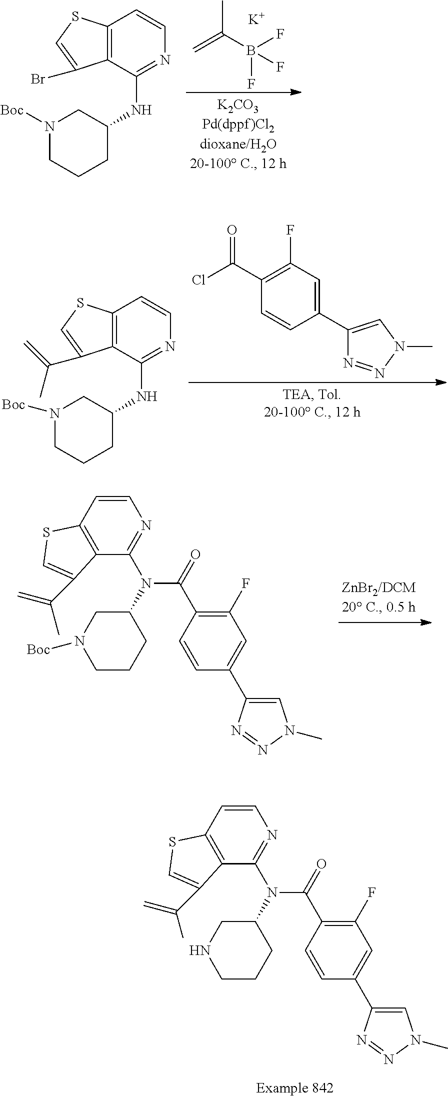

### Step 1:

To a solution of tert-butyl (3R)-3-[(3-bromothieno[3,2-c]pyridin-4-yl)amino]piperidine-1-carboxylate (100 mg, 242.52 μmol) in dioxane (2 mL) was added potassium trifluoro(isopropenyl)boranuide (71.77 mg, 485.03 μmol), K₂CO₃ (100.55 mg, 727.55 μmol), Pd(dppf)Cl₂ (17.75 mg, 24.25 μmol) and H₂O (0.4 mL) at 20° C. under N₂ atmosphere, then the mixture was stirred at 100° C. for 12 h. The reaction mixture was diluted water (2 mL) and then extracted with EtOAc (3×5 mL). The combined organic layer was washed with brine (3×5 mL), dried over Na₂SO₄, filtered and concentrated under reduced pressure to give a residue. The residue was purified by prep-TLC (SiO₂, petroleum ether:EtOAc=2:1) to give tert-butyl (R)-3-((3-(prop-1-en-2-yl)thieno[3,2-c]pyridin-4-yl)amino)piperidine-1-carboxylate (83 mg, 86% yield) as an off-white solid. LC-MS: [M+H⁺]=374.0.

### Step 2:

To a solution of 2-fluoro-4-(1-methyltriazol-4-yl)benzoyl chloride (106.50 mg, 444.43 μmol) in toluene (3 mL) was added tert-butyl (R)-3-((3-(prop-1-en-2-yl)thieno[3,2-c]pyridin-4-yl)amino)piperidine-1-carboxylate (83 mg, 222.22 μmol) and TEA (179.89 mg, 1.78 mmol, 247.44 μL) at 20° C. under N₂ atmosphere, then the mixture was stirred at 110° C. for 12 h. The reaction mixture was added water (2 mL) and then extracted with EtOAc (3×5 mL). The combined organic layer was washed with brine (3×5 mL), dried over Na₂SO₄, filtered and concentrated under reduced pressure to give a residue. The residue was purified by prep-TLC (SiO₂, EtOAc:methanol=3:1) to give tert-butyl (R)-3-(2-fluoro-4-(1-methyl-1H-1,2,3-triazol-4-yl)-N-(3-(prop-1-en-2-yl)thieno[3,2-c]pyridin-4-yl)benzamido)piperidine-1-carboxylate (74 mg, 35% yield) was obtained as an off-white solid. LC-MS: [M+H⁺]=577.3.

### Step 3:

A solution of tert-butyl (R)-3-(2-fluoro-4-(1-methyl-1H-1,2,3-triazol-4-yl)-N-(3-(prop-1-en-2-yl)thieno[3,2-c]pyridin-4-yl)benzamido)piperidine-1-carboxylate (74 mg, 128.32 μmol) in DCM (2 mL) was added ZnBr₂ (144.49 mg, 641.60 μmol, 32.11 μL) at 20° C. under N₂ atmosphere, then the mixture was stirred at 20° C. for 1 h. The reaction mixture was filtered and concentrated under reduced pressure to give the title compound (23 mg, 36% yield) as a white solid. LC-MS: [M+H⁺]=477.3. ¹H NMR (400 MHZ, DMSO-d₆): δ ppm 8.75-8.36 (m, 1H), 8.27-7.95 (m, 1H), 7.92-7.76 (m, 1H), 7.74-7.65 (m, 1H), 7.38-7.26 (m, 1H), 7.08-6.90 (m, 1H), 5.49-5.41 (m, 1H), 5.15-4.99 (m, 1H), 4.19-4.01 (m, 1H), 4.02 (d, J=2.8 Hz, 2H), 3.96-3.80 (m, 1H), 3.53 (br d, J=11.0 Hz, 1H), 3.05-2.84 (m, 2H), 2.21 (s, 1H), 2.14 (br d, J=3.1 Hz, 3H), 1.93-1.73 (m, 1H), 1.71-1.40 (m, 2H), 1.37-0.78 (m, 1H).

### Synthesis of (R)—N-(8-methylisoquinolin-1-yl)-N-(piperidin-3-yl)-6-(pyridin-2-ylamino)nicotinamide (Example 843)

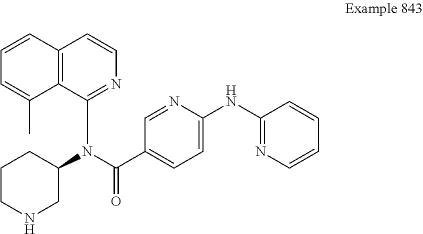 [SMILES: Cc1cccc2ccnc(N(C(=O)c3ccc(Nc4ccccn4)nc3)[C@@H]3CCCNC3)c12]

Example 843 was synthesized following the procedure for example 131 by substituting 2-flouro-4-iodobenzoyl chloride with 6-bromonicotinoyl chloride and 3-amino-1-methyl-pyridin-2-one with pyridin-2-amine to afford the title compound as an off-white solid. LC-MS: [M+H⁺]=439.20. ¹H NMR (CD₃OD, 400 MHZ): δ 8.55 (d, J=5.6 Hz, 1H), 8.24 (d, J=6.4 Hz, 1H), 8.20-8.13 (m, 2H), 7.89 (d, J=5.2 Hz, 1H), 7.76-7.74 (m, 1H), 7.57-7.51 (m, 2H), 7.44-7.43 (m, 1H), 7.32-7.27 (m, 2H), 6.80 (d, J=8.8 Hz, 1H), 5.12-5.03 (m, 1H), 3.97-3.94 (m, 1H), 3.79-3.73 (m, 1H), 3.47-3.36 (m, 1H), 2.97-2.95 (m, 1H), 2.75 (s, 3H), 1.94-1.79 (m, 3H), 1.41-1.28 (m, 1H).

### Synthesis of (R)-4-(3H-[1,2,3]triazolo[4,5-b]pyridin-3-yl)-2-fluoro-N-(2-(5-hydroxypent-1-yn-1-yl)thieno[3,2-c]pyridin-4-yl)-N-(piperidin-3-yl)benzamide (Example 844)

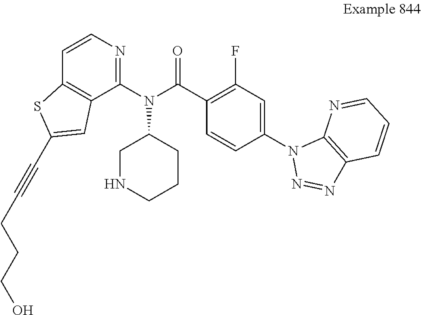 [SMILES: O=C(c1ccc(-n2nnc3cccnc32)cc1F)N(c1nccc2sc(C#CCCCO)cc12)[C@@H]1CCCNC1]

Example 844 was prepared according to the procedure described for Example 818 by substituting tert-butyl (R)-3-(N-(2-bromothieno[3,2-c]pyridin-4-yl)-2-fluoro-4-(5-methyl-1,3,4-thiadiazol-2-yl)benzamido)piperidine-1-carboxylate with tert-butyl (R)-3-(4-(3H-[1,2,3]triazolo[4,5-b]pyridin-3-yl)-N-(2-bromothieno[3,2-c]pyridin-4-yl)-2-fluorobenzamido)piperidine-1-carboxylate and but-3-yn-1-ol with pent-4-yn-1-ol, provided the title compound as off white solid. LC-MS: [M+H⁺]=556.1.

### Synthesis of 2-fluoro-N-[8-methyl-6-(1-methylpyrazol-4-yl)-1-isoquinolyl]-4-(1-methyltriazol-4-yl)-N-[(3R)-3-piperidyl]benzamide (Example 845)

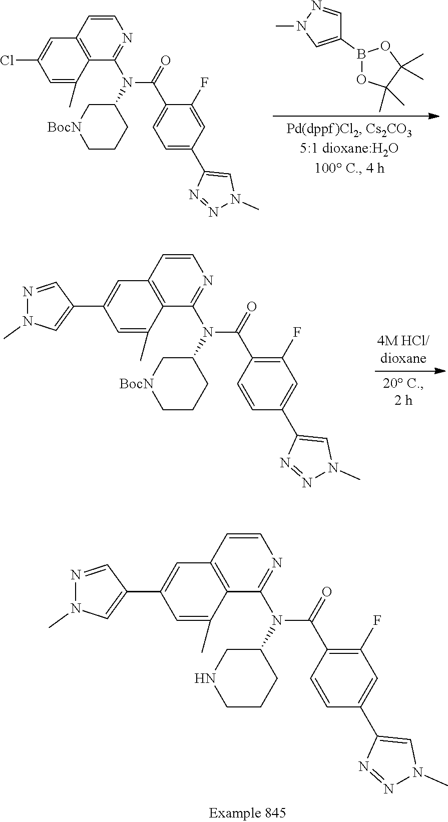

### Step 1:

To a mixture of tert-butyl (3R)-3-[(6-chloro-8-methyl-1-isoquinolyl)-[2-fluoro-4-(1-methyltriazol-4-yl)benzoyl]amino]piperidine-1-carboxylate (30 mg, 51.81 μmol), 1-methyl-4-(4,4,5,5-tetramethyl-1,3,2-dioxaborolan-2-yl)pyrazole (16.17 mg, 77.71 μmol), and Cs₂CO₃ (50.64 mg, 155.42 μmol) in dioxane (1 mL) and H₂O (0.2 mL) at 20° C. was added Pd(dppf)Cl₂ (3.79 mg, 5.18 μmol) under N₂. The mixture was heated to 100° C. and stirred for 4 h under N₂. The reaction mixture was filtered and concentrated. The residue was purified by prep-TLC (SiO₂, EtOAc:Methanol=10:1) to give tert-butyl (3R)-3-[[2-fluoro-4-(1-methyltriazol-4-yl)benzoyl]-[8-methyl-6-(1-methylpyrazol-4-yl)-1-isoquinolyl]amino]piperidine-1-carboxylate (145 mg, 66%) as a white solid. LC-MS: [M+H⁺]=625.4.

### Step 2:

Removal the Boc protecting group according to the procedure described for Example 97 provided the title compound hydrochloride salt as off-white solid. LC-MS: [M+H⁺]=525.4. ¹H NMR (400 MHZ, CD₃OD): δ=8.41-8.33 (m, 1H), 8.17-8.10 (m, 2H), 8.01-7.96 (m, 1H), 7.82-7.76 (m, 1H), 7.74-7.64 (m, 2H), 7.27 (dd, J=1.2, 11.3 Hz, 1H), 7.17-7.13 (m, 1H), 6.97-6.85 (m, 1H), 5.13-5.00 (m, 1H), 4.04 (s, 3H), 4.01 (br dd, J=4.1, 7.9 Hz, 1H), 3.94 (s, 3H), 3.87-3.74 (m, 1H), 3.41 (br d, J=12.6 Hz, 1H), 3.08-2.96 (m, 1H), 2.90-2.83 (m, 3H), 2.07-1.81 (m, 3H), 1.72-1.50 (m, 1H).

### Synthesis of (R)—N-(8-methylisoquinolin-1-yl)-N-(piperidin-3-yl)-6-(pyrimidin-2-ylamino)nicotinamide (Example 846)

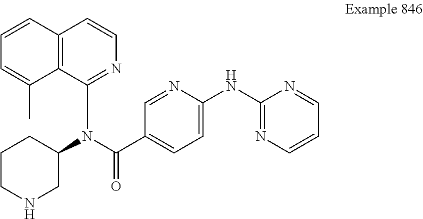 [SMILES: Cc1cccc2ccnc(N(C(=O)c3ccc(Nc4ncccn4)nc3)[C@@H]3CCCNC3)c12]

Example 846 was synthesized following the procedure for example 131 by substituting 2-flouro-4-iodobenzoyl chloride with 6-bromonicotinoyl chloride and 3-amino-1-methyl-pyridin-2-one with 2-aminopyrimidine to afford the title compound as an off-white solid. LC-MS: [M+H⁺]=440.2. 1H NMR (CD₃OD, 400 MHZ): δ 8.73 (d, J=5.2 Hz, 2H), 8.58 (d, J=5.2 Hz, 1H), 8.01-8.00 (m, 1H), 7.95-7.89 (m, 2H), 7.82-7.79 (m, 1H), 7.62-7.84 (m, 2H), 7.30 (t, J=5.2 Hz, 1H), 7.13 (d, J=9.2 Hz, 1H), 5.11-5.03 (m, 1H), 3.98-3.95 (m, 1H), 3.76-3.70 (m, 1H), 3.41-3.35 (m, 1H), 2.96-2.92 (m, 1H), 2.74 (s, 3H), 1.93-1.79 (m, 3H), 1.34-1.17 (m, 1H).

### Synthesis of (R)-3-chloro-N-(8-methylisoquinolin-1-yl)-N-(piperidin-3-yl)-4-(pyrimidin-2-ylamino)benzamide (Example 847)

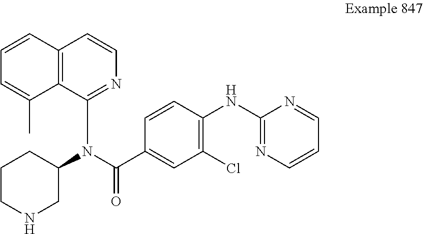 [SMILES: Cc1cccc2ccnc(N(C(=O)c3ccc(Nc4ncccn4)c(Cl)c3)[C@@H]3CCCNC3)c12]

Example 847 was synthesized following the procedure for example 131 by substituting 2-flouro-4-iodobenzoylchloride with 3-chloro-4-iodobenzoyl chloride and 3-amino-1-methyl-pyridin-2-one with 2-aminopyrimidine to afford the title compound as an off-white solid. LC-MS: [M+H⁺]=474.05. ¹H NMR (CD₃OD, 400 MHZ): δ 8.58-8.56 (m, 2H), 8.53-8.48 (m, 1H), 7.87-7.82 (m, 1H), 7.80-7.71 (m, 2H), 7.54-7.50 (m, 1H), 7.45-7.41 (m, 1H), 7.14-7.13 (m, 1H), 7.09-7.03 (m, 2H), 5.11-5.03 (m, 1H), 3.95-3.92 (m, 1H), 3.81-3.75 (m, 1H), 3.47-3.40 (m, 1H), 3.00-2.95 (m, 1H), 2.75 (s, 3H), 2.69-2.65 (m, 1H), 1.94-1.81 (m, 2H), 1.45-1.43 (m, 1H).

### Synthesis of (R)-4-((4-aminopyrimidin-2-yl)amino)-2-fluoro-N-(8-methylisoquinolin-1-yl)-N-(piperidin-3-yl)benzamide (Example 848)

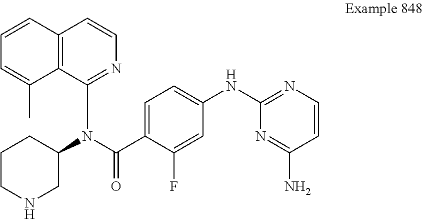 [SMILES: Cc1cccc2ccnc(N(C(=O)c3ccc(Nc4nccc(N)n4)cc3F)[C@@H]3CCCNC3)c12]

Example 848 was synthesized following the procedure for example 210 by substituting 2-chloropyrimidine with tert-butyl (2-chloropyrimidin-4-yl)carbamate to afford the title compound as a yellow solid. LC-MS: [M+H⁺]=472.2. ¹H NMR (CD₃OD, 400 MHz): δ 8.43-8.39 (m, 1H), 7.75-7.72 (m, 1H), 7.70-7.64 (m, 2H), 7.53-7.49 (m, 1H), 7.48-7.42 (m, 2H), 6.84-6.77 (m, 2H), 6.21 (d, J=7.6 Hz, 1H), 5.07-5.02 (m, 1H), 3.98-3.95 (m, 1H), 3.82-3.76 (m, 1H), 3.59-3.57 (m, 1H), 3.40-3.34 (m, 1H), 2.81 (s, 3H), 2.69-2.68 (m, 1H), 1.98-1.87 (m, 2H), 1.55-1.50 (m, 1H).

### Synthesis of (R)-4-(3H-[1,2,3]triazolo[4,5-b]pyridin-3-yl)-2-fluoro-N-(2,6-naphthyridin-1-yl)-N-(piperidin-3-yl)benzamide (Example 849)

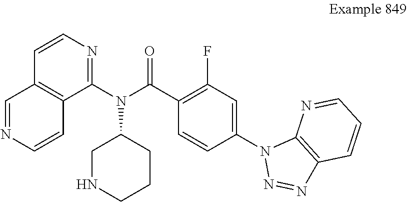 [SMILES: O=C(c1ccc(-n2nnc3cccnc32)cc1F)N(c1nccc2cnccc12)[C@@H]1CCCNC1]

Example 849 was synthesized following the procedure for Example 300 by substituting 1-chloro-2,7-naphthyridine with 1-chloro-2,6-naphthyridine, providing the title compound as an off-white solid. LC-MS: [M+H⁺]=469.2. ¹H NMR (CD₃OD, 400 MHZ): δ 9.25 (bs, 1H), 8.68-8.63 (m, 3H), 8.41 (d, J=8.8 Hz, 1H), 8.02-7.84 (m, 4H), 7.45-7.42 (m, 1H), 7.28-7.26 (m, 1H), 5.21-5.19 (m, 1H), 3.90-3.86 (m, 1H), 3.64-3.61 (m, 1H), 2.80-2.77 (m, 1H), 1.95-1.84 (m, 3H), 1.20-1.18 (m, 2H).

### Synthesis of 2-fluoro-N-(6-fluoro-8-methyl-1-isoquinolyl)-4-[1-(2-methoxyethyl)triazol-4-yl]-N-[(3R)-3-piperidyl]benzamide (Example 850)

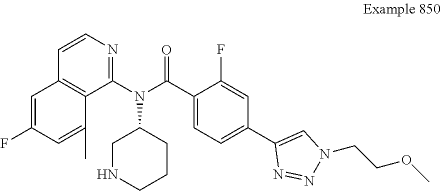 [SMILES: COCCn1cc(-c2ccc(C(=O)N(c3nccc4cc(F)cc(C)c34)[C@@H]3CCCNC3)c(F)c2)nn1]

Example 850 was prepared according to the procedure described for Example 127 by substituting tert-butyl (R)-3-((8-methylisoquinolin-1-yl)amino)piperidine-1-carboxylate with tert-butyl (R)-3-((6-fluoro-8-methylisoquinolin-1-yl)amino)piperidine-1-carboxylate and 2-bromo-5-methyl-1,3,4-thiadiazole with 4-bromo-1-(2-methoxyethyl)-1H-1,2,3-triazole, provided the title compound as a white solid. LC-MS: [M+H]=507.4. ¹H NMR (400 MHZ, DMSO-d₆): δ=9.29-9.11 (m, 1H), 9.10-8.83 (m, 1H), 8.50-8.48 (m, 1H), 8.48-8.43 (m, 1H), 7.82-7.75 (m, 1H), 7.58-7.43 (m, 2H), 7.41-7.31 (m, 1H), 7.24 (dd, J=1.2, 8.1 Hz, 1H), 6.88-6.78 (m, 1H), 4.98-4.74 (m, 1H), 4.49 (t, J=5.1 Hz, 2H), 3.83-3.75 (m, 2H), 3.69 (br t, J=5.1 Hz, 3H), 3.61-3.49 (m, 1H), 3.29-3.22 (m, 1H), 3.21 (s, 3H), 2.83-2.77 (m, 3H), 2.45-2.36 (m, 1H), 2.04-1.91 (m, 1H), 1.89-1.70 (m, 3H), 1.53 (d, J=6.9 Hz, 1H), 1.41-1.26 (m, 1H).

### Synthesis of 2-fluoro-4-[5-(methoxymethyl)-1-methyl-triazol-4-yl]-N-(3-methylthieno[3,2-c]pyridin-4-yl)-N-[(3R)-3-piperidyl]benzamide (Example 851)

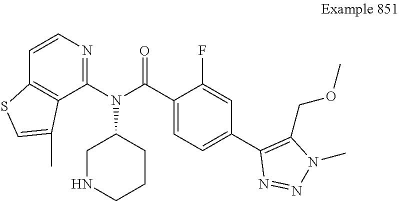 [SMILES: COCc1c(-c2ccc(C(=O)N(c3nccc4scc(C)c34)[C@@H]3CCCNC3)c(F)c2)nnn1C]

Example 850 was prepared according to the procedure described for Example 127 by substituting tert-butyl (R)-3-((8-methylisoquinolin-1-yl)amino)piperidine-1-carboxylate with tert-butyl (R)-3-((3-methylthieno[3,2-c]pyridin-4-yl)amino)piperidine-1-carboxylate and 2-bromo-5-methyl-1,3,4-thiadiazole with 4-bromo-5-(methoxymethyl)-1-methyl-1H-1,2,3-triazole, provided the title compound as yellow solid. LC-MS: [M+H+]=495.2. ¹H NMR (400 MHZ, CD₃OD): δ=8.35-8.27 (m, 1H), 7.83 (d, J=5.5 Hz, 1H), 7.42-7.36 (m, 1H), 7.20 (d, J=10.5 Hz, 1H), 7.16-7.07 (m, 2H), 4.77-4.69 (m, 1H), 4.48 (s, 2H), 4.06 (s, 3H), 3.69 (br d, J=11.5 Hz, 1H), 3.31 (s, 3H), 3.00-2.90 (m, 1H), 2.64-2.59 (m, 3H), 2.52-2.39 (m, 1H), 2.34-2.20 (m, 1H), 1.80-1.56 (m, 3H), 1.17-1.03 (m, 1H).

### Synthesis of 2-fluoro-N-(6-fluoro-8-methyl-1-isoquinolyl)-4-[5-(2-methoxyethyl)-1,3,4-thiadiazol-2-yl]-N-[(3R)-3-piperidyl]benzamide (Example 852)

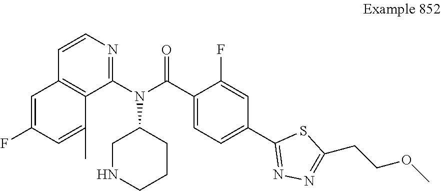 [SMILES: COCCc1nnc(-c2ccc(C(=O)N(c3nccc4cc(F)cc(C)c34)[C@@H]3CCCNC3)c(F)c2)s1]

Example 852 was prepared according to the procedure described for Example 127 by substituting tert-butyl (R)-3-((8-methylisoquinolin-1-yl)amino)piperidine-1-carboxylate with tert-butyl (R)-3-((6-fluoro-8-methylisoquinolin-1-yl)amino)piperidine-1-carboxylate and 2-bromo-5-methyl-1,3,4-thiadiazole with 2-bromo-5-(2-methoxyethyl)-1,3,4-thiadiazole, provided the title compound as white solid. LC-MS: [M+H]=524.3.

¹H NMR (400 MHZ, DMSO-d₆): δ=9.47-9.23 (m, 1H), 9.22-8.97 (m, 1H), 8.49-8.40 (m, 1H), 7.83-7.78 (m, 1H), 7.59-7.47 (m, 3H), 7.36 (dd, J=1.3, 8.1 Hz, 1H), 7.01-6.90 (m, 1H), 4.97-4.87 (m, 1H), 4.86-4.69 (m, 2H), 3.84-3.69 (m, 1H), 3.63 (t, J=5.9 Hz, 2H), 3.59-3.48 (m, 1H), 3.33-3.29 (m, 2H), 3.26 (s, 3H), 2.83 (s, 1H), 2.80 (s, 2H), 2.47-2.34 (m, 1H), 1.85-1.75 (m, 2H), 1.39-1.25 (m, 1H).

### Synthesis of 2-fluoro-N-(6-fluoro-8-methyl-1-isoquinolyl)-4-[5-(methoxymethyl)-1,3,4-thiadiazol-2-yl]-N-[(3R)-3-piperidyl]benzamide (Example 853)

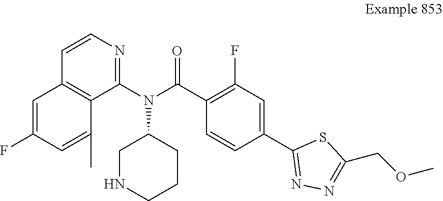 [SMILES: COCc1nnc(-c2ccc(C(=O)N(c3nccc4cc(F)cc(C)c34)[C@@H]3CCCNC3)c(F)c2)s1]

Example 853 was prepared according to the procedure described for Example 127 by substituting tert-butyl (R)-3-((8-methylisoquinolin-1-yl)amino)piperidine-1-carboxylate with tert-butyl (R)-3-((6-fluoro-8-methylisoquinolin-1-yl)amino)piperidine-1-carboxylate and 2-bromo-5-methyl-1,3,4-thiadiazole with 2-bromo-5-(methoxymethyl)-1,3,4-thiadiazole, provided the title compound as a white solid. LC-MS: [M+H]=510.3. ¹H NMR (400 MHZ, DMSO-d6): δ=9.28-8.96 (m, 2H), 8.96-8.79 (m, 1H), 8.49-8.42 (m, 1H), 7.81 (d, J=5.5 Hz, 1H), 7.61-7.49 (m, 3H), 7.41 (dd, J=1.4, 8.1 Hz, 1H), 7.03-6.94 (m, 1H), 4.96-4.87 (m, 1H), 4.83 (s, 2H), 3.84-3.77 (m, 1H), 3.60-3.48 (m, 1H), 3.45 (s, 1H), 3.36 (s, 3H), 3.26 (br d, J=12.5 Hz, 1H), 2.99-2.88 (m, 1H), 2.84-2.80 (m, 3H), 1.81 (br d, J=9.8 Hz, 2H), 1.53 (d, J=7.0 Hz, 1H), 1.36-1.22 (m, 1H).

### Synthesis of 4-[5-(ethoxymethyl)-1,3,4-thiadiazol-2-yl]-2-fluoro-N-(6-fluoro-8-methyl-1-isoquinolyl)-N-[(3R)-3-piperidyl]benzamide (Example 854)

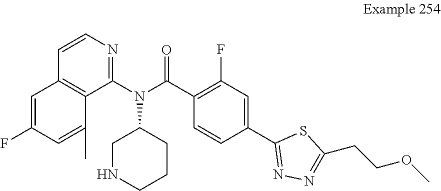 [SMILES: COCCc1nnc(-c2ccc(C(=O)N(c3nccc4cc(F)cc(C)c34)[C@@H]3CCCNC3)c(F)c2)s1]

Example 854 was prepared according to the procedure described for Example 127 by substituting tert-butyl (R)-3-((8-methylisoquinolin-1-yl)amino)piperidine-1-carboxylate with tert-butyl (R)-3-((6-fluoro-8-methylisoquinolin-1-yl)amino)piperidine-1-carboxylate and 2-bromo-5-methyl-1,3,4-thiadiazole with 2-bromo-5-(ethoxymethyl)-1,3,4-thiadiazole, provided the title compound as a brown solid. LC-MS: [M+H⁺]=524.3. ¹H NMR (400 MHZ, DMSO-d₆): δ=9.25-8.96 (m, 2H), 8.95-8.76 (m, 1H), 8.50-8.41 (m, 1H), 7.86-7.77 (m, 1H), 7.61-7.48 (m, 3H), 7.41 (dd, J=1.3, 8.1 Hz, 1H), 7.05-6.94 (m, 1H), 5.00-4.89 (m, 1H), 4.87 (s, 2H), 3.81 (br d, J=11.5 Hz, 1H), 3.57 (q, J=6.9 Hz, 2H), 2.86-2.78 (m, 3H), 1.89-1.73 (m, 2H), 1.53 (d, J=7.0 Hz, 1H), 1.42-1.26 (m, 1H), 1.16 (br d, J=1.3 Hz, 3H), 1.07 (s, 1H).

### Synthesis of N-(5-chloro-8-methyl-1-isoquinolyl)-4-(1-methyltriazol-4-yl)-N-[(3R)-3-piperidyl]benzamide (Example 855)

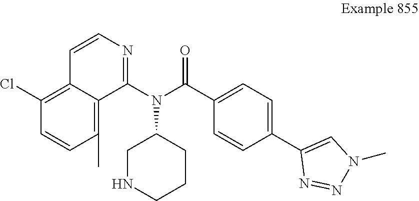 [SMILES: Cc1ccc(Cl)c2ccnc(N(C(=O)c3ccc(-c4cn(C)nn4)cc3)[C@@H]3CCCNC3)c12]

Example 855 was prepared according to the procedure described for Example 111 by substituting tert-butyl (R)-3-((8-methylisoquinolin-1-yl)amino)piperidine-1-carboxylate with tert-butyl (R)-3-((5-chloro-8-methylisoquinolin-1-yl)amino)piperidine-1-carboxylate, provided the title compound as a white solid. LC-MS: [M+H]=461.1. ¹H NMR (400 MHZ, DMSO-d₆): δ=9.03-8.83 (m, 2H), 8.72-8.65 (m, 1H), 8.40 (s, 1H), 8.13-8.05 (m, 1H), 7.73 (d, J=7.8 Hz, 1H), 7.50-7.36 (m, 3H), 7.04-6.94 (m, 2H), 5.05-4.89 (m, 1H), 4.00 (s, 3H), 3.78 (br d, J=11.5 Hz, 1H), 3.60-3.54 (m, 1H), 3.30-3.23 (m, 1H), 2.87-2.79 (m, 1H), 2.67 (s, 3H), 1.88-1.73 (m, 2H), 1.73-1.61 (m, 1H), 1.33-1.22 (m, 1H).

### Synthesis of N-(5-chloro-8-methyl-1-isoquinolyl)-4-(5-methyl-1,3,4-thiadiazol-2-yl)-N-[(3R)-3-piperidyl]benzamide (Example 856)

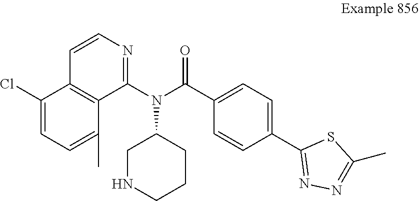 [SMILES: Cc1nnc(-c2ccc(C(=O)N(c3nccc4c(Cl)ccc(C)c34)[C@@H]3CCCNC3)cc2)s1]

Example 856 was prepared according to the procedure described for Example 111 by substituting tert-butyl (R)-3-((8-methylisoquinolin-1-yl)amino)piperidine-1-carboxylate with tert-butyl (R)-3-((5-chloro-8-methylisoquinolin-1-yl)amino)piperidine-1-carboxylate and 4-bromo-1-methyl-1H-1,2,3-triazole with 2-bromo-5-methyl-1,3,4-thiadiazole, provided the title compound as yellow solid. LC-MS: [M+H⁺]=478.2. ¹H NMR (400 MHZ, CD₃OD): δ=8.65 (d, J=5.8 Hz, 1H), 8.19 (d, J=5.8 Hz, 1H), 7.61 (d, J=7.8 Hz, 1H), 7.55 (d, J=8.4 Hz, 2H), 7.34 (d, J=8.0 Hz, 1H), 7.12 (d, J=8.4 Hz, 2H), 5.16-5.04 (m, 1H), 4.00-3.93 (m, 1H), 3.87-3.77 (m, 1H), 3.40 (br d, J=13.3 Hz, 1H), 2.99 (dt, J=4.4, 12.3 Hz, 1H), 2.81-2.77 (m, 1H), 2.74 (s, 6H), 2.02-1.81 (m, 3H), 1.54 (dq, J=4.9, 12.2 Hz, 1H).

### Synthesis of N-(5-chloro-8-methyl-1-isoquinolyl)-5-(5-methyl-1,3,4-thiadiazol-2-yl)-N-[(3R)-3-piperidyl]pyridine-2-carboxamide (Example 857)

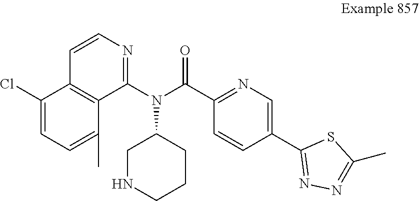 [SMILES: Cc1nnc(-c2ccc(C(=O)N(c3nccc4c(Cl)ccc(C)c34)[C@@H]3CCCNC3)nc2)s1]

Example 857 was prepared according to the procedure described for Example 127 by substituting tert-butyl (R)-3-((8-methylisoquinolin-1-yl)amino)piperidine-1-carboxylate with tert-butyl (R)-3-((5-chloro-8-methylisoquinolin-1-yl)amino)piperidine-1-carboxylate and 2-fluoro-4-iodobenzoyl chloride with 5-bromopicolinoyl chloride, provided the title compound as white solid. LC-MS: [M+H+]=479.2. ¹H NMR (400 MHz, CD₃OD): δ=8.46 (d, J=6.0 Hz, 1H), 8.37 (br d, J=5.1 Hz, 1H), 8.36-8.27 (m, 2H), 8.05 (d, J=8.1 Hz, 1H), 7.81 (d, J=7.8 Hz, 1H), 7.57-7.44 (m, 1H), 5.07-4.93 (m, 1H), 4.01 (br d, J=11.9 Hz, 1H), 3.53 (br t, J=11.5 Hz, 1H), 2.96-2.83 (m, 2H), 2.79 (d, J=6.3 Hz, 6H), 1.96-1.74 (m, 3H), 1.16-0.98 (m, 1H).

### Synthesis of N-(6-methoxy-8-methyl-1-isoquinolyl)-4-(1-methyltriazol-4-yl)-N-[(3R)-3-piperidyl]benzamide (Example 858)

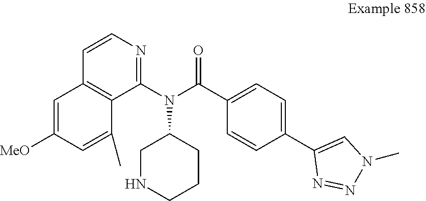 [SMILES: COc1cc(C)c2c(N(C(=O)c3ccc(-c4cn(C)nn4)cc3)[C@@H]3CCCNC3)nccc2c1]

Example 858 was prepared according to the procedure described for Example 111 by substituting tert-butyl (R)-3-((8-methylisoquinolin-1-yl)amino)piperidine-1-carboxylate with tert-butyl (R)-3-((6-methoxy-8-methylisoquinolin-1-yl)amino)piperidine-1-carboxylate, provided the title compound as a white solid. LC-MS: [M+H⁺]=457.3. ¹H NMR (400 MHZ, CD₃OD): δ=8.44-8.35 (m, 1H), 8.22 (s, 1H), 7.83 (br s, 1H), 7.48 (br d, J=8.0 Hz, 2H), 7.25-7.05 (m, 4H), 5.08-4.96 (m, 1H), 4.10 (s, 3H), 3.97 (br d, J=12.0 Hz, 1H), 3.91-3.74 (m, 4H), 3.50-3.37 (m, 1H), 2.99 (dt, J=5.0, 12.0 Hz, 1H), 2.82-2.67 (m, 3H), 2.01-1.87 (m, 2H), 1.80 (br d, J=11.5 Hz, 1H), 1.55-1.41 (m, 1H).

### Synthesis of 4-(1-ethyltriazol-4-yl)-N-(3-methylthieno[3,2-c]pyridin-4-yl)-N-[(3R)-3-piperidyl] benzamide (Example 859)

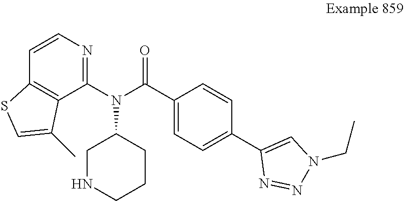 [SMILES: CCn1cc(-c2ccc(C(=O)N(c3nccc4scc(C)c34)[C@@H]3CCCNC3)cc2)nn1]

Example 859 was prepared according to the procedure described for Example 111 by substituting tert-butyl (R)-3-((8-methylisoquinolin-1-yl)amino)piperidine-1-carboxylate with tert-butyl (R)-3-((3-methylthieno[3,2-c]pyridin-4-yl)amino)piperidine-1-carboxylate and 4-bromo-1-methyl-1H-1,2,3-triazole with 4-bromo-1-ethyl-1H-1,2,3-triazole, provided the title compound as gray solid. LC-MS: [M+H]=447.2. ¹H NMR (400 MHZ, CD₃OD): δ=8.45 (d, J=5.50 Hz, 1H), 8.27 (s, 1H), 7.96 (d, J=5.50 Hz, 1H), 7.52 (d, J=8.38 Hz, 2H), 7.31 (s, 1H), 7.23 (d, J=8.38 Hz, 2H), 5.08-5.24 (m, 1H), 4.43 (q, J=7.38 Hz, 2H), 3.85-3.95 (m, 1H), 3.69-3.80 (m, 1H), 2.85-3.01 (m, 1H), 2.39-2.61 (m, 3H), 1.83-1.96 (m, 2H), 1.74-1.83 (m, 1H), 1.52 (t, J=7.32 Hz, 3H), 1.21-1.38 (m, 2H).

### Synthesis of 2-fluoro-N-(6-methoxy-8-methyl-1-isoquinolyl)-4-(5-methyl-1,3,4-thiadiazol-2-yl)-N-[(3R)-3-piperidyl]benzamide (Example 860)

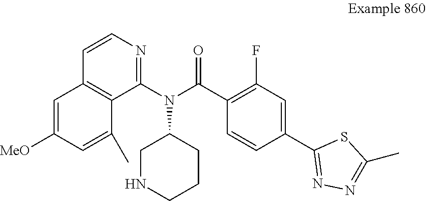 [SMILES: COc1cc(C)c2c(N(C(=O)c3ccc(-c4nnc(C)s4)cc3F)[C@@H]3CCCNC3)nccc2c1]

Example 860 was prepared according to the procedure described for Example 127 by substituting tert-butyl (R)-3-((8-methylisoquinolin-1-yl)amino)piperidine-1-carboxylate with tert-butyl (R)-3-((6-methoxy-8-methylisoquinolin-1-yl)amino)piperidine-1-carboxylate, provided the title compound as a white solid. LC-MS: [M+H⁺]=492.3. ¹H NMR (400 MHZ, CD3OD-d4): δ=8.33 (d, J=5.8 Hz, 1H), 7.73 (d, J=5.8 Hz, 1H), 7.47 (dd, J=1.4, 10.7 Hz, 1H), 7.37-7.30 (m, 1H), 7.19-7.03 (m, 3H), 5.06-4.95 (m, 1H), 4.06-3.97 (m, 1H), 3.88-3.77 (m, 4H), 3.40 (br d, J=12.4 Hz, 1H), 3.09-2.94 (m, 1H), 2.86-2.73 (m, 6H), 2.05-1.79 (m, 3H), 1.66-1.46 (m, 1H).

### Synthesis of N-(5,8-dimethyl-1-isoquinolyl)-4-(1-methyltriazol-4-yl)-N-[(3R)-3-piperidyl]benzamide (Example 861)

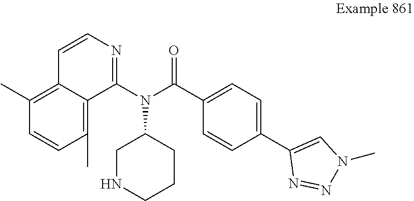

Example 861 was prepared according to the procedure described for Example 111 by substituting tert-butyl (R)-3-((8-methylisoquinolin-1-yl)amino)piperidine-1-carboxylate with tert-butyl (R)-3-((5,8-dimethylisoquinolin-1-yl)amino)piperidine-1-carboxylate, provided the title compound as a white solid. LC-MS: [M+H]=441.2. ¹H NMR (400 MHZ, CD₃OD): δ=8.57-8.50 (m, 1H), 8.13 (s, 1H), 7.96 (d, J=5.8 Hz, 1H), 7.43-7.38 (m, 2H), 7.34-7.31 (m, 1H), 7.28-7.23 (m, 1H), 7.10-7.05 (m, 2H), 5.08 (br d, J=1.5 Hz, 1H), 4.07 (s, 3H), 3.99-3.92 (m, 1H), 3.85-3.77 (m, 1H), 3.37 (br s, 1H), 3.03-2.94 (m, 1H), 2.77-2.70 (m, 3H), 2.55-2.52 (m, 3H), 1.96-1.87 (m, 2H), 1.86-1.79 (m, 1H), 1.53-1.41 (m, 1H).

### Synthesis of N-(5,6-difluoro-8-methyl-1-isoquinolyl)-4-(1-methyltriazol-4-yl)-N-[(3R)-3-piperidyl]benzamide (Example 862)

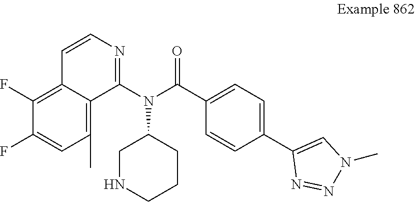 [SMILES: Cc1cc(F)c(F)c2ccnc(N(C(=O)c3ccc(-c4cn(C)nn4)cc3)[C@@H]3CCCNC3)c12]

Example 862 was prepared according to the procedure described for Example 111 by substituting tert-butyl (R)-3-((8-methylisoquinolin-1-yl)amino)piperidine-1-carboxylate with tert-butyl (R)-3-((5,6-difluoro-8-methylisoquinolin-1-yl)amino)piperidine-1-carboxylate, provided the title compound as a white solid. LC-MS: [M+H]=463.2. ¹H NMR (400 MHz, CD₃OD): δ=8.63 (d, J=5.65 Hz, 1H), 8.18 (s, 1H), 8.01 (d, J=5.65 Hz, 1H), 7.46 (d, J=8.41 Hz, 2H), 7.34 (br dd, J=11.23, 8.22 Hz, 1H), 7.03-7.10 (m, 2H), 5.06-5.17 (m, 1H), 4.08 (s, 3H), 3.91-3.98 (m, 1H), 3.78-3.87 (m, 1H), 3.40 (br d, J=12.80 Hz, 2H), 2.99 (td, J=12.39, 4.58 Hz, 1H), 2.69-2.81 (m, 4H), 1.90-2.02 (m, 2H), 1.80-1.89 (m, 1H), 1.49-1.60 (m, 1H).

### Synthesis of 4-(1-ethyltriazol-4-yl)-2-fluoro-N-(6-methoxy-8-methyl-1-isoquinolyl)-N-[(3R)-3-piperidyl] benzamide (Example 863)

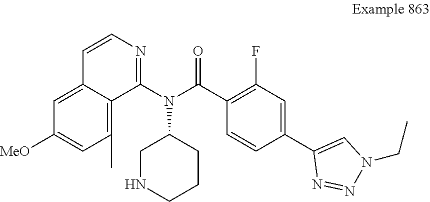 [SMILES: CCn1cc(-c2ccc(C(=O)N(c3nccc4cc(OC)cc(C)c34)[C@@H]3CCCNC3)c(F)c2)nn1]

Example 863 was prepared according to the procedure described for Example 127 by substituting tert-butyl (R)-3-((8-methylisoquinolin-1-yl)amino)piperidine-1-carboxylate with tert-butyl (R)-3-((6-methoxy-8-methylisoquinolin-1-yl)amino)piperidine-1-carboxylate and 2-bromo-5-methyl-1,3,4-thiadiazole with 4-bromo-1-ethyl-1H-1,2,3-triazole, provided the title compound as a white solid. LC-MS: [M+H⁺]=489.3. ¹H NMR (400 MHZ, CD₃OD): δ=8.37-8.30 (m, 1H), 8.27 (s, 1H), 7.67 (d, J=5.5 Hz, 1H), 7.36-7.26 (m, 1H), 7.20 (d, J=8.0 Hz, 1H), 7.12 (s, 1H), 7.05 (d, J=2.3 Hz, 1H), 6.94 (t, J=7.6 Hz, 1H), 5.08-4.98 (m, 1H), 4.43 (q, J=7.4 Hz, 2H), 4.05-3.97 (m, 1H), 3.91-3.76 (m, 4H), 3.41 (br d, J=12.6 Hz, 1H), 3.08-2.98 (m, 1H), 2.86-2.77 (m, 3H), 2.24-1.84 (m, 3H), 1.66-1.57 (m, 1H), 1.53 (t, J=7.4 Hz, 3H).

### Synthesis of N-(6-methoxy-8-methyl-1-isoquinolyl)-5-(5-methyl-1,3,4-thiadiazol-2-yl)-N-[(3R)-3-piperidyl]pyridine-2-carboxamide (Example 864)

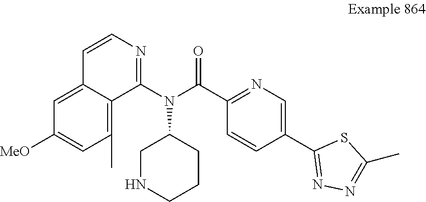 [SMILES: COc1cc(C)c2c(N(C(=O)c3ccc(-c4nnc(C)s4)cn3)[C@@H]3CCCNC3)nccc2c1]

Example 864 was prepared according to the procedure described for Example 127 by substituting tert-butyl (R)-3-((8-methylisoquinolin-1-yl)amino)piperidine-1-carboxylate with tert-butyl (R)-3-((6-methoxy-8-methylisoquinolin-1-yl)amino)piperidine-1-carboxylate and 2-fluoro-4-iodobenzoyl chloride with 5-bromopicolinoyl chloride, provided the title compound as a white solid. LC-MS: [M+H⁺]=475.3. ¹H NMR (400 MHZ, CD₃OD): δ=8.43 (d, J=1.9 Hz, 1H), 8.34-8.22 (m, 2H), 8.06-7.93 (m, 1H), 7.87 (br d, J=1.1 Hz, 1H), 7.26 (br s, 1H), 7.24-7.13 (m, 1H), 5.02-4.92 (m, 1H), 4.03-3.91 (m, 4H), 3.55-3.47 (m, 1H), 3.35 (br s, 1H), 2.99-2.82 (m, 2H), 2.81-2.69 (m, 5H), 1.94-1.77 (m, 3H), 1.21-1.07 (m, 1H).

### Synthesis of N-(6-ethoxy-8-methyl-1-isoquinolyl)-5-(5-methyl-1,3,4-thiadiazol-2-yl)-N-[(3R)-3-piperidyl]pyridine-2-carboxamide (Example 865)

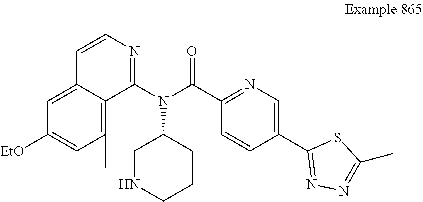 [SMILES: CCOc1cc(C)c2c(N(C(=O)c3ccc(-c4nnc(C)s4)cn3)[C@@H]3CCCNC3)nccc2c1]

Example 865 was prepared according to the procedure described for Example 127 by substituting tert-butyl (R)-3-((8-methylisoquinolin-1-yl)amino)piperidine-1-carboxylate with tert-butyl (R)-3-((6-ethoxy-8-methylisoquinolin-1-yl)amino)piperidine-1-carboxylate and 2-fluoro-4-iodobenzoyl chloride with 5-bromopicolinoyl chloride, provided the title compound as a yellow solid. LC-MS: [M+H⁺]=489.3. ¹H NMR (400 MHZ, CD₃OD): δ=8.46-8.35 (m, 2H), 8.31 (dd, J=2.5, 6.0 Hz, 1H), 8.24-8.15 (m, 1H), 8.14-8.02 (m, 1H), 7.41 (br s, 1H), 7.34-7.24 (m, 1H), 5.04-4.92 (m, 1H), 4.32-4.19 (m, 2H), 4.11-3.99 (m, 1H), 3.52-3.41 (m, 1H), 3.36 (br s, 1H), 2.92 (dt, J=5.5, 11.4 Hz, 1H), 2.79 (s, 3H), 2.68 (s, 3H), 1.98-1.82 (m, 2H), 1.75 (br d, J=11.6 Hz, 1H), 1.46 (t, J=6.9 Hz, 3H), 1.17-1.01 (m, 1H).

### Synthesis of N-(6-methoxy-8-methyl-1-isoquinolyl)-4-(5-methyl-1,3,4-thiadiazol-2-yl)-N-[(3R)-3-piperidyl]benzamide (Example 866)

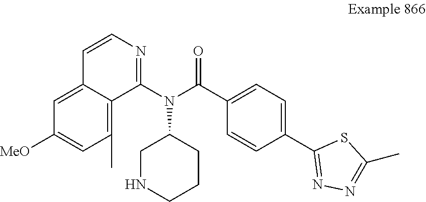 [SMILES: COc1cc(C)c2c(N(C(=O)c3ccc(-c4nnc(C)s4)cc3)[C@@H]3CCCNC3)nccc2c1]

Example 866 was prepared according to the procedure described for Example 127 by substituting tert-butyl (R)-3-((8-methylisoquinolin-1-yl)amino)piperidine-1-carboxylate with tert-butyl (R)-3-((6-methoxy-8-methylisoquinolin-1-yl)amino)piperidine-1-carboxylate and 2-fluoro-4-iodobenzoyl chloride with 4-bromobenzoyl chloride, provided the title compound as a white solid. LC-MS: [M+H⁺]=474.3. ¹H NMR (400 MHZ, CD₃OD): δ=8.42 (d, J=5.9 Hz, 1H), 7.91 (d, J=5.9 Hz, 1H), 7.63 (d, J=8.3 Hz, 2H), 7.30 (d, J=8.3 Hz, 2H), 7.18 (br d, J=12.3 Hz, 2H), 5.01 (br s, 1H), 4.08-3.96 (m, 1H), 3.88 (s, 3H), 3.86-3.79 (m, 1H), 3.39 (br s, 1H), 3.09-2.95 (m, 1H), 2.75 (d, J=12.6 Hz, 6H), 2.04-1.70 (m, 3H), 1.49 (br dd, J=4.9, 11.5 Hz, 1H).

### Synthesis of N-(5-fluoro-6-methoxy-1-isoquinolyl)-5-(5-methyl-1,3,4-thiadiazol-2-yl)-N-[(3R)-3-piperidyl]pyridine-2-carboxamide (Example 867)

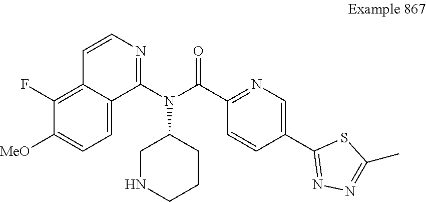 [SMILES: COc1ccc2c(N(C(=O)c3ccc(-c4nnc(C)s4)cn3)[C@@H]3CCCNC3)nccc2c1F]

Example 867 was prepared according to the procedure described for Example 127 by substituting tert-butyl (R)-3-((8-methylisoquinolin-1-yl)amino)piperidine-1-carboxylate with tert-butyl (R)-3-((5-fluoro-6-methoxyisoquinolin-1-yl)amino)piperidine-1-carboxylate and 2-fluoro-4-iodobenzoyl chloride with 5-bromopicolinoyl chloride, provided the title compound as a white solid. LC-MS: [M+H⁺]=479.3. ¹H NMR (400 MHZ, CD₃OD): δ=8.44-8.23 (m, 3H), 8.15-8.06 (m, 1H), 8.06-7.98 (m, 2H), 7.82-7.58 (m, 1H), 5.26-5.04 (m, 1H), 4.11-4.04 (m, 3H), 3.98-3.84 (m, 1H), 3.78-3.66 (m, 1H), 3.40-3.33 (m, 1H), 2.99-2.85 (m, 1H), 2.82-2.74 (m, 3H), 2.44-2.19 (m, 1H), 1.99-1.82 (m, 2H), 1.61-1.39 (m, 1H).

### Synthesis of N-(5-fluoro-6-methoxy-1-isoquinolyl)-4-(5-methyl-1,3,4-thiadiazol-2-yl)-N-[(3R)-3-piperidyl]benzamide (Example 868)

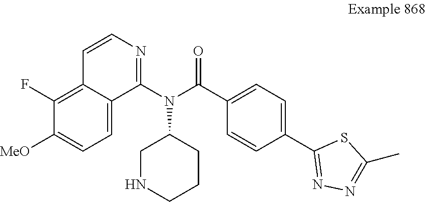 [SMILES: COc1ccc2c(N(C(=O)c3ccc(-c4nnc(C)s4)cc3)[C@@H]3CCCNC3)nccc2c1F]

Example 868 was prepared according to the procedure described for Example 127 by substituting tert-butyl (R)-3-((8-methylisoquinolin-1-yl)amino)piperidine-1-carboxylate with tert-butyl (R)-3-((5-fluoro-6-methoxyisoquinolin-1-yl)amino)piperidine-1-carboxylate and 2-fluoro-4-iodobenzoyl chloride with 4-bromobenzoyl chloride, provided the title compound as yellow solid. LC-MS: [M+H⁺]=478.2. ¹H NMR (400 MHZ, CD3OD): δ=8.44 (br s, 1H), 7.95-7.82 (m, 2H), 7.62 (br d, J=6.1 Hz, 3H), 7.39 (br s, 2H), 5.24 (br d, J=1.6 Hz, 1H), 4.01 (br s, 3H), 3.95-3.85 (m, 1H), 3.71 (br d, J=9.9 Hz, 1H), 3.36 (br s, 1H), 2.87 (br s, 1H), 2.76 (s, 3H), 2.03-1.79 (m, 3H), 1.41-1.24 (m, 1H).

### Synthesis of N-(5-chloro-6-methoxy-1-isoquinolyl)-4-(1-methyltriazol-4-yl)-N-[(3R)-3-piperidyl]benzamide (Example 869)

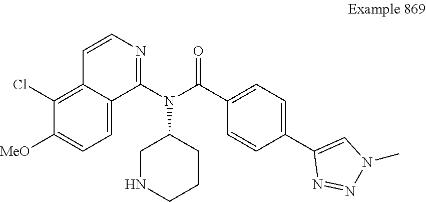 [SMILES: COc1ccc2c(N(C(=O)c3ccc(-c4cn(C)nn4)cc3)[C@@H]3CCCNC3)nccc2c1Cl]

Example 869 was prepared according to the procedure described for Example 111 by substituting tert-butyl (R)-3-((8-methylisoquinolin-1-yl)amino)piperidine-1-carboxylate with tert-butyl (R)-3-((5-chloro-6-methoxyisoquinolin-1-yl)amino)piperidine-1-carboxylate, provided the title compound as yellow solid. LC-MS: [M+H⁺]=477.2. ¹H NMR (400 MHz, CD3OD-d4): δ=8.49 (br d, J=4.0 Hz, 1H), 8.19 (s, 1H), 8.04 (br d, J=8.1 Hz, 1H), 8.00 (d, J=6.0 Hz, 1H), 7.58 (br d, J=7.4 Hz, 1H), 7.48 (br d, J=7.1 Hz, 2H), 7.31 (br s, 2H), 5.26 (br s, 1H), 4.08 (s, 3H), 4.02 (br s, 3H), 3.93-3.84 (m, 1H), 3.77-3.65 (m, 1H), 3.36 (br s, 1H), 2.87 (br s, 1H), 2.02-1.80 (m, 3H), 1.37-1.24 (m, 1H).

### Synthesis of N-(5-methoxy-8-methyl-1-isoquinolyl)-5-(5-methyl-1,3,4-thiadiazol-2-yl)-N-[(3R)-3-piperidyl]pyridine-2-carboxamide (Example 870)

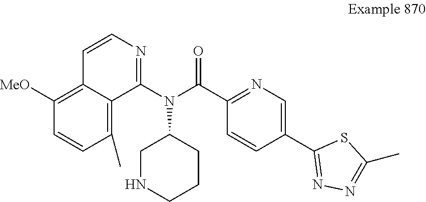 [SMILES: COc1ccc(C)c2c(N(C(=O)c3ccc(-c4nnc(C)s4)cn3)[C@@H]3CCCNC3)nccc12]

Example 870 was prepared according to the procedure described for Example 127 by substituting tert-butyl (R)-3-((8-methylisoquinolin-1-yl)amino)piperidine-1-carboxylate with tert-butyl (R)-3-((5-methoxy-8-methylisoquinolin-1-yl)amino)piperidine-1-carboxylate and 2-fluoro-4-iodobenzoyl chloride with 5-bromopicolinoyl chloride, provided the title compound as a white solid. LC-MS: [M+H+]=475.2. ¹H NMR (400 MHZ, CD₃OD-d4): δ=8.40-8.34 (m, 2H), 8.33-8.26 (m, 2H), 8.04-7.95 (m, 1H), 7.44 (d, J=8.0 Hz, 1H), 7.21-7.07 (m, 1H), 5.04-4.91 (m, 1H), 4.01 (s, 4H), 3.57-3.48 (m, 1H), 2.97-2.81 (m, 2H), 2.80-2.76 (m, 3H), 2.68 (s, 3H), 1.91-1.76 (m, 3H), 1.17-0.96 (m, 1H).

### Synthesis of N-(5-methoxy-8-methyl-1-isoquinolyl)-4-(5-methyl-1,3,4-thiadiazol-2-yl)-N-[(3R)-3-piperidyl]benzamide (Example 871)

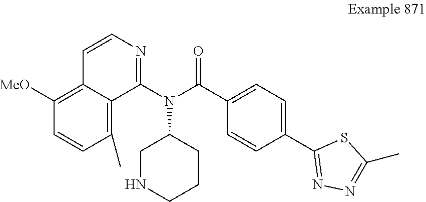 [SMILES: COc1ccc(C)c2c(N(C(=O)c3ccc(-c4nnc(C)s4)cc3)[C@@H]3CCCNC3)nccc12]

Example 871 was prepared according to the procedure described for Example 127 by substituting tert-butyl (R)-3-((8-methylisoquinolin-1-yl)amino)piperidine-1-carboxylate with tert-butyl (R)-3-((5-methoxy-8-methylisoquinolin-1-yl)amino)piperidine-1-carboxylate and 2-fluoro-4-iodobenzoyl chloride with 4-bromobenzoyl chloride, provided the title compound as a yellow solid. LC-MS: [M+H⁺]=474.3. ¹H NMR (400 MHZ, CD3OD): δ=8.53-8.45 (m, 1H), 8.19-8.10 (m, 1H), 7.56 (d, J=8.5 Hz, 2H), 7.33-7.27 (m, 1H), 7.17 (d, J=8.5 Hz, 2H), 6.93 (d, J=8.1 Hz, 1H), 5.11-5.01 (m, 1H), 3.96 (br d, J=12.0 Hz, 1H), 3.92-3.87 (m, 3H), 3.83-3.74 (m, 1H), 3.38 (br d, J=12.6 Hz, 1H), 3.04-2.92 (m, 1H), 2.76-2.64 (m, 6H), 1.98-1.78 (m, 3H), 1.51-1.37 (m, 1H).

### Synthesis of N-(5-methoxy-8-methyl-1-isoquinolyl)-4-(1-methyltriazol-4-yl)-N-[(3R)-3-piperidyl]benzamide (Example 872)

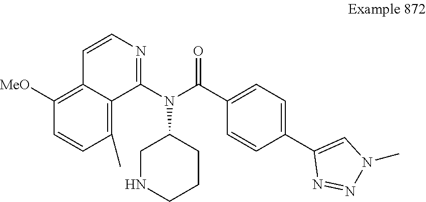 [SMILES: COc1ccc(C)c2c(N(C(=O)c3ccc(-c4cn(C)nn4)cc3)[C@@H]3CCCNC3)nccc12]

Example 872 was prepared according to the procedure described for Example 111 by substituting tert-butyl (R)-3-((8-methylisoquinolin-1-yl)amino)piperidine-1-carboxylate with tert-butyl (R)-3-((5-methoxy-8-methylisoquinolin-1-yl)amino)piperidine-1-carboxylate, provided the title compound as a green solid. LC-MS: [M+H]=457.3. ¹H NMR (400 MHZ, CD3OD-d4): δ=8.52-8.46 (m, 1H), 8.18-8.11 (m, 2H), 7.45-7.39 (m, 2H), 7.31-7.26 (m, 1H), 7.12-7.05 (m, 2H), 6.92 (d, J=8.1 Hz, 1H), 5.12-5.01 (m, 1H), 4.07 (s, 3H), 3.98-3.91 (m, 1H), 3.91-3.88 (m, 3H), 3.83-3.75 (m, 1H), 3.42-3.38 (m, 1H), 3.03-2.92 (m, 1H), 2.71-2.65 (m, 3H), 1.98-1.87 (m, 2H), 1.86-1.78 (m, 1H), 1.50-1.38 (m, 1H).

### Synthesis of N-(8-fluoro-5-methoxy-1-isoquinolyl)-4-(1-methyltriazol-4-yl)-N-[(3R)-3-piperidyl] benzamide (Example 873)

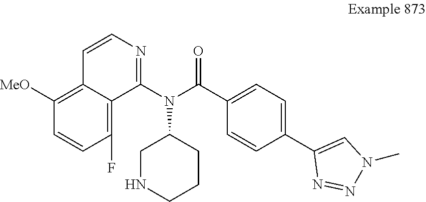 [SMILES: COc1ccc(F)c2c(N(C(=O)c3ccc(-c4cn(C)nn4)cc3)[C@@H]3CCCNC3)nccc12]

Example 874 was prepared according to the procedure described for Example 111 by substituting tert-butyl (R)-3-((8-methylisoquinolin-1-yl)amino)piperidine-1-carboxylate with tert-butyl (R)-3-((8-fluoro-5-methoxyisoquinolin-1-yl)amino)piperidine-1-carboxylate, provided the title compound as a white solid. LC-MS: [M+H⁺]=461.2. ¹H NMR (400 MHZ, CD₃OD): δ=8.58-8.52 (m, 1H), 8.17 (s, 1H), 8.09-8.04 (m, 1H), 7.50-7.42 (m, 2H), 7.27-7.14 (m, 3H), 7.04-6.95 (m, 1H), 5.28-5.13 (m, 1H), 4.08 (s, 3H), 3.95-3.82 (m, 4H), 3.80-3.67 (m, 1H), 3.41-3.33 (m, 1H), 2.99-2.83 (m, 1H), 2.00-1.81 (m, 3H), 1.42 (dq, J=5.6, 12.5 Hz, 1H).

### Synthesis of 2-fluoro-N-(6-fluoro-5-methoxy-1-isoquinolyl)-4-(1-methyltriazol-4-yl)-N-[(3R)-3-piperidyl] benzamide (Example 874)

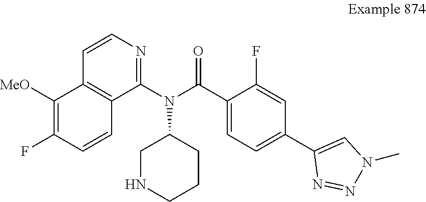 [SMILES: COc1c(F)ccc2c(N(C(=O)c3ccc(-c4cn(C)nn4)cc3F)[C@@H]3CCCNC3)nccc12]

Example 874 was prepared according to the procedure described for Example 127 by substituting tert-butyl (R)-3-((8-methylisoquinolin-1-yl)amino)piperidine-1-carboxylate with tert-butyl (R)-3-((6-fluoro-5-methoxyisoquinolin-1-yl)amino)piperidine-1-carboxylate and 2-bromo-5-methyl-1,3,4-thiadiazole with 4-bromo-1-methyl-1H-1,2,3-triazole, provided the title compound as a yellow solid. LC-MS: [M+H⁺]=479.3. ¹H NMR (400 MHZ, CD3OD): δ=8.47-8.38 (m, 1H), 8.18 (br s, 1H), 8.01-7.85 (m, 2H), 7.62-7.47 (m, 1H), 7.33-7.03 (m, 3H), 5.27-5.16 (m, 1H), 4.10-4.01 (m, 6H), 3.96-3.87 (m, 1H), 3.82-3.52 (m, 2H), 2.96-2.79 (m, 1H), 2.04-1.78 (m, 3H), 1.33-1.17 (m, 1H).

### Synthesis of 4-[5-(methoxymethyl)-1,3,4-thiadiazol-2-yl]-N-(3-methylthieno[3,2-c]pyridin-4-yl)-N-[(3R)-3-piperidyl]benzamide (Example 875)

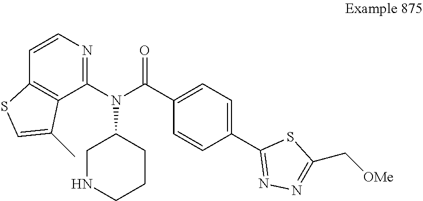 [SMILES: COCc1nnc(-c2ccc(C(=O)N(c3nccc4scc(C)c34)[C@@H]3CCCNC3)cc2)s1]

Example 875 was prepared according to the procedure described for Example 127 by substituting tert-butyl (R)-3-((8-methylisoquinolin-1-yl)amino)piperidine-1-carboxylate with tert-butyl (R)-3-((3-methylthieno[3,2-c]pyridin-4-yl)amino)piperidine-1-carboxylate; 2-fluoro-4-iodobenzoyl chloride with 4-bromobenzoyl chloride; 2-bromo-5-methyl-1,3,4-thiadiazole with 2-bromo-5-(methoxymethyl)-1,3,4-thiadiazole, provided the title compound as off-white solid. LC-MS: [M+H]=480.2. ¹H NMR (400 MHZ, CD3OD-d4) δ=8.41 (br d, J=5.5 Hz, 1H), 7.97 (d, J=5.5 Hz, 1H), 7.69 (d, J=8.4 Hz, 2H), 7.35-7.31 (m, 3H), 5.15 (tt, J=3.8, 11.6 Hz, 1H), 4.83 (s, 2H), 3.92 (br dd, J=3.3, 12.1 Hz, 1H), 3.79-3.70 (m, 1H), 3.46 (s, 3H), 3.37 (br d, J=12.5 Hz, 1H), 2.97-2.89 (m, 1H), 2.50 (d, J=0.9 Hz, 3H), 1.94-1.87 (m, 2H), 1.84-1.77 (m, 1H), 1.33-1.26 (m, 1H).

### Synthesis of 4-[5-(3-methoxypropyl)-1,3,4-thiadiazol-2-yl]-N-(3-methylthieno[3,2-c]pyridin-4-yl)-N-[(3R)-3-piperidyl]benzamide (Example 876)

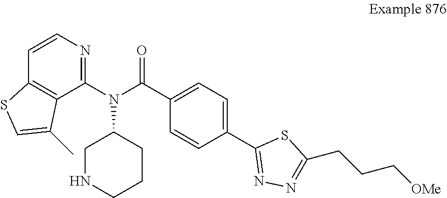 [SMILES: COCCCc1nnc(-c2ccc(C(=O)N(c3nccc4scc(C)c34)[C@@H]3CCCNC3)cc2)s1]

Example 876 was prepared according to the procedure described for Example 127 by substituting tert-butyl (R)-3-((8-methylisoquinolin-1-yl)amino)piperidine-1-carboxylate with tert-butyl (R)-3-((3-methylthieno[3,2-c]pyridin-4-yl)amino)piperidine-1-carboxylate; 2-fluoro-4-iodobenzoyl chloride with 4-bromobenzoyl chloride; 2-bromo-5-methyl-1,3,4-thiadiazole with 2-bromo-5-(3-methoxypropyl)-1,3,4-thiadiazole, provided the title compound as a brown solid. LC-MS: [M+H]=508.2. ¹H NMR (400 MHZ, CD3OD-d₄): δ=8.45 (d, J=5.5 Hz, 1H), 7.97 (d, J=5.4 Hz, 1H), 7.66 (d, J=8.4 Hz, 2H), 7.36-7.28 (m, 3H), 5.21-5.08 (m, 1H), 3.92 (br d, J=9.9 Hz, 1H), 3.80-3.65 (m, 1H), 3.45 (t, J=6.1 Hz, 2H), 3.35 (br s, 3H), 3.18 (t, J=7.5 Hz, 2H), 2.98-2.88 (m, 1H), 2.57 (s, 1H), 2.50 (s, 3H), 2.08-1.98 (m, 2H), 1.96-1.75 (m, 3H), 1.34-1.24 (m, 1H).

### Synthesis of N-(5,8-dimethyl-1-isoquinolyl)-5-(5-methyl-1,3,4-thiadiazol-2-yl)-N-[(3R)-3-piperidyl]pyridine-2-carboxamide (Example 877)

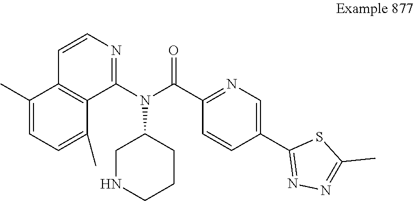

Example 877 was prepared according to the procedure described for Example 127 by substituting tert-butyl (R)-3-((8-methylisoquinolin-1-yl)amino)piperidine-1-carboxylate with tert-butyl (R)-3-((5,8-dimethylisoquinolin-1-yl)amino)piperidine-1-carboxylate and 2-fluoro-4-iodobenzoyl chloride with 5-bromopicolinoyl chloride, provided the title compound as a white solid. LC-MS: [M+H⁺]=459.2. ¹H NMR (400 MHZ, CD3OD-d4): δ=8.38 (d, J=1.6 Hz, 1H), 8.34-8.24 (m, 1H), 8.24-8.14 (m, 1H), 7.92-7.87 (m, 1H), 7.86-7.75 (m, 1H), 7.47-7.38 (m, 1H), 7.37-7.29 (m, 1H), 4.70-4.58 (m, 1H), 3.72 (br d, J=12.0 Hz, 1H), 3.10 (dd, J=10.6, 11.8 Hz, 1H), 3.00-2.84 (m, 2H), 2.83-2.74 (m, 5H), 2.60 (s, 3H), 2.45-2.30 (m, 1H), 1.86-1.53 (m, 3H), 0.96-0.81 (m, 1H).

### Synthesis of N-(5,8-dimethyl-1-isoquinolyl)-4-(5-methyl-1,3,4-thiadiazol-2-yl)-N-[(3R)-3-piperidyl]benzamide (Example 878)

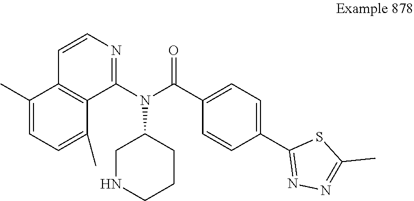

Example 878 was prepared according to the procedure described for Example 127 by substituting tert-butyl (R)-3-((8-methylisoquinolin-1-yl)amino)piperidine-1-carboxylate with tert-butyl (R)-3-((5,8-dimethylisoquinolin-1-yl)amino)piperidine-1-carboxylate and 2-fluoro-4-iodobenzoyl chloride with 4-bromobenzoyl chloride, provided the title compound as a yellow solid. LC-MS: [M+H+]=458.2. ¹H NMR (400 MHZ, CD3OD-d4): δ=8.58-8.50 (m, 1H), 7.99-7.91 (m, 1H), 7.55 (d, J=8.4 Hz, 2H), 7.37-7.26 (m, 2H), 7.17 (d, J=8.4 Hz, 2H), 5.13-5.02 (m, 1H), 3.97 (br d, J=11.9 Hz, 1H), 3.86-3.77 (m, 1H), 3.39 (br d, J=12.8 Hz, 1H), 3.06-2.93 (m, 1H), 2.79-2.70 (m, 6H), 2.57-2.51 (m, 3H), 1.99-1.79 (m, 3H), 1.54-1.42 (m, 1H).

### Synthesis of 4-[5-(2-cyanoethyl)-1,3,4-thiadiazol-2-yl]-2-fluoro-N-(8-methyl-1-isoquinolyl)-N-[(3R)-3-piperidyl]benzamide (Example 879)

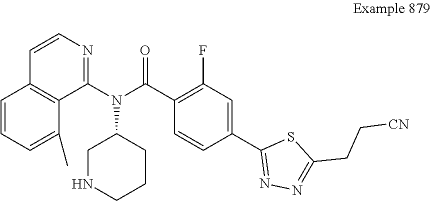 [SMILES: Cc1cccc2ccnc(N(C(=O)c3ccc(-c4nnc(CCC#N)s4)cc3F)[C@@H]3CCCNC3)c12]

Example 879 was prepared according to the procedure described for Example 127 by substituting 2-bromo-5-methyl-1,3,4-thiadiazole with 3-(5-bromo-1,3,4-thiadiazol-2-yl)propanenitrile, provided the title compound as a white solid. LC-MS: [M+H]=501.2. ¹H NMR (400 MHz, METHANOL-d4): δ=8.45-8.40 (m, 1H), 7.78-7.70 (m, 1H), 7.68-7.61 (m, 1H), 7.55-7.47 (m, 2H), 7.46-7.40 (m, 1H), 7.28 (dd, J=1.6, 8.1 Hz, 1H), 7.03-6.96 (m, 1H), 5.07 (tt, J=3.5, 11.4 Hz, 1H), 4.02 (br dd, J=3.4, 12.1 Hz, 1H), 3.89-3.78 (m, 1H), 3.46 (t, J=7.0 Hz, 2H), 3.41 (br d, J=12.8 Hz, 1H), 2.99 (t, J=7.0 Hz, 3H), 2.85 (s, 3H), 2.01-1.90 (m, 3H), 1.68-1.51 (m, 1H).

### Synthesis of 4-[5-(2-cyanoethyl)-1,3,4-thiadiazol-2-yl]-N-(8-methyl-1-isoquinolyl)-N-[(3R)-3-piperidyl]benzamide (Example 880)

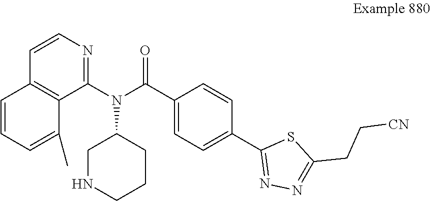 [SMILES: Cc1cccc2ccnc(N(C(=O)c3ccc(-c4nnc(CCC#N)s4)cc3)[C@@H]3CCCNC3)c12]

Example 880 was prepared according to the procedure described for Example 127 by substituting 2-fluoro-4-iodobenzoyl chloride with 4-bromobenzoyl chloride and 2-bromo-5-methyl-1,3,4-thiadiazole with 3-(5-bromo-1,3,4-thiadiazol-2-yl) propanenitrile, provided the title compound as off white solid. LC-MS: [M+H]=483.3. ¹H NMR (400 MHz, CD3OD): δ=8.50-8.44 (m, 1H), 7.78-7.72 (m, 1H), 7.65 (br d, J=8.0 Hz, 1H), 7.56 (d, J=8.4 Hz, 2H), 7.46 (t, J=7.7 Hz, 1H), 7.42-7.36 (m, 1H), 7.24-7.16 (m, 2H), 4.82-4.72 (m, 1H), 3.75-3.67 (m, 1H), 3.45 (t, J=7.1 Hz, 2H), 3.38 (br d, J=10.4 Hz, 1H), 3.09-3.03 (m, 1H), 3.01-2.97 (m, 2H), 2.81-2.75 (m, 3H), 2.61-2.50 (m, 1H), 1.78-1.54 (m, 3H), 1.37-1.27 (m, 1H).

### Synthesis of 4-[1-(2-cyanoethyl)triazol-4-yl]-2-fluoro-N-(3-methylthieno[3,2-c]pyridin-4-yl)-N-[(3R)-3-piperidyl]benzamide (Example 881)

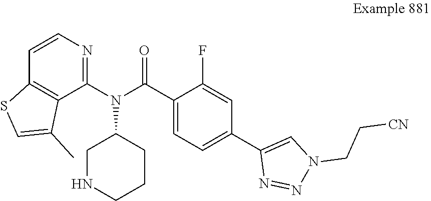 [SMILES: Cc1csc2ccnc(N(C(=O)c3ccc(-c4cn(CCC#N)nn4)cc3F)[C@@H]3CCCNC3)c12]

Example 881 was prepared according to the procedure described for Example 888 by substituting tert-butyl (R)-3-((8-methylisoquinolin-1-yl)amino)piperidine-1-carboxylate with tert-butyl (R)-3-((3-methylthieno[3,2-c]pyridin-4-yl)amino)piperidine-1-carboxylate, provided the title compound as brown solid. LC-MS (M+H)=490.3. ¹H NMR (400 MHZ, CD₃OD): δ ppm 8.39-8.33 (m, 2H), 7.90 (d, J=5.5 Hz, 1H), 7.45-7.36 (m, 2H), 7.27 (dd, J=1.4, 8.1 Hz, 1H), 7.04 (t, J=7.6 Hz, 1H), 5.14-5.02 (m, 1H), 4.69 (t, J=6.4 Hz, 2H), 4.01-3.93 (m, 1H), 3.77-3.68 (m, 1H), 3.35 (br s, 1H), 3.15 (s, 2H), 2.98-2.90 (m, 1H), 2.66-2.58 (m, 3H), 1.97-1.82 (m, 3H), 1.34-1.28 (m, 1H).

### Synthesis of 4-[5-(2-cyanoethyl)-1,3,4-thiadiazol-2-yl]-N-(3-methylthieno[3,2-c]pyridin-4-yl)-N-[(3R)-3-piperidyl]benzamide (Example 882)

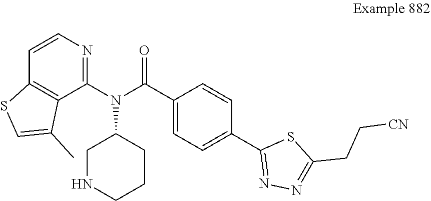 [SMILES: Cc1csc2ccnc(N(C(=O)c3ccc(-c4nnc(CCC#N)s4)cc3)[C@@H]3CCCNC3)c12]

Example 882 was prepared according to the procedure described for Example 127 by substituting tert-butyl (R)-3-((8-methylisoquinolin-1-yl)amino)piperidine-1-carboxylate with tert-butyl (R)-3-((3-methylthieno[3,2-c]pyridin-4-yl)amino)piperidine-1-carboxylate; 2-fluoro-4-iodobenzoyl chloride with 4-bromobenzoyl chloride; 2-bromo-5-methyl-1,3,4-thiadiazole with 3-(5-bromo-1,3,4-thiadiazol-2-yl)propanenitrile, provided the title compound as an off white solid. LC-MS: [M+H]=489.1. ¹H NMR (400 MHZ, CD₃OD): δ ppm 8.45 (d, J=5.4 Hz, 1H), 7.97 (d, J=5.5 Hz, 1H), 7.67 (d, J=8.5 Hz, 2H), 7.38-7.27 (m, 3H), 5.21-5.08 (m, 1H), 3.97-3.87 (m, 1H), 3.80-3.68 (m, 1H), 3.47 (t, J=7.0 Hz, 2H), 3.37-3.32 (m, 2H), 3.01 (t, J=7.0 Hz, 2H), 2.62-2.44 (m, 3H), 1.98-1.73 (m, 3H), 1.30 (br dd, J=4.8, 11.6 Hz, 1H).

### Synthesis of 4-[5-(2-cyanoethyl)-1,3,4-thiadiazol-2-yl]-2-fluoro-N-(3-methylthieno[3,2-c]pyridin-4-yl)-N-[(3R)-3-piperidyl]benzamide (Example 883)

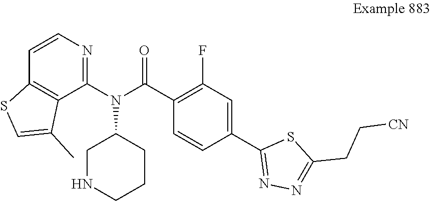 [SMILES: Cc1csc2ccnc(N(C(=O)c3ccc(-c4nnc(CCC#N)s4)cc3F)[C@@H]3CCCNC3)c12]

Example 883 was prepared according to the procedure described for Example 127 by substituting tert-butyl (R)-3-((8-methylisoquinolin-1-yl)amino)piperidine-1-carboxylate with tert-butyl (R)-3-((3-methylthieno[3,2-c]pyridin-4-yl)amino)piperidine-1-carboxylate and 2-bromo-5-methyl-1,3,4-thiadiazole with 3-(5-bromo-1,3,4-thiadiazol-2-yl)propanenitrile, provided the title compound as a light yellow solid. LC-MS (M+H)=507.2. ¹H NMR (400 MHZ, METHANOL-d₄): δ ppm 8.37 (d, J=5.5 Hz, 1H), 7.92 (d, J=5.5 Hz, 1H), 7.58-7.53 (m, 1H), 7.45 (s, 1H), 7.43-7.37 (m, 1H), 7.16 (t, J=7.5 Hz, 1H), 5.11-5.03 (m, 1H), 3.98 (br dd, J=3.3, 11.8 Hz, 1H), 3.71 (t, J=11.5 Hz, 1H), 3.48 (t, J=7.0 Hz, 2H), 3.38 (br s, 1H), 3.01 (t, J=7.0 Hz, 2H), 2.93 (br dd, J=5.3, 9.1 Hz, 1H), 2.61 (s, 3H), 1.95-1.83 (m, 3H), 1.37-1.23 (m, 1H).

### Synthesis of N-(6-chloro-8-methyl-1-isoquinolyl)-6-(1-methyltriazol-4-yl)-N-[(3R)-3-piperidyl]pyridine-3-carboxamide (Example 884)

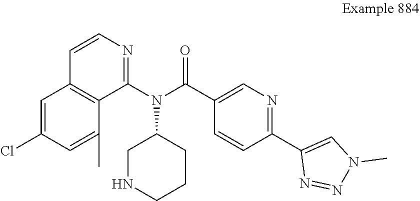 [SMILES: Cc1cc(Cl)cc2ccnc(N(C(=O)c3ccc(-c4cn(C)nn4)nc3)[C@@H]3CCCNC3)c12]

Example 884 was prepared according to the procedure described for Example 111 by substituting tert-butyl (R)-3-((8-methylisoquinolin-1-yl)amino)piperidine-1-carboxylate with tert-butyl (R)-3-((6-chloro-8-methylisoquinolin-1-yl)amino)piperidine-1-carboxylate and 4-(methoxycarbonyl)phenyl)boronic acid with 3-Methyl 6-borono-3-pyridinecarboxylate, provided the title compound as off white solid. LC-MS: [M+H]=462.2. ¹H NMR (400 MHZ, CD₃OD): δ ppm 8.56 (d, J=5.5 Hz, 1H), 8.27 (s, 1H), 8.14 (d, J=1.6 Hz, 1H), 7.83-7.76 (m, 2H), 7.69 (d, J=8.3 Hz, 1H), 7.57-7.50 (m, 1H), 7.42 (s, 1H), 5.15-5.01 (m, 1H), 4.10 (s, 3H), 3.99-3.91 (m, 1H), 3.86-3.73 (m, 1H), 3.41 (br s, 1H), 3.03-2.93 (m, 1H), 2.73 (s, 3H), 2.01-1.79 (m, 3H), 1.58-1.43 (m, 1H).

### Synthesis of 6-(1-ethyltriazol-4-yl)-N-(3-methylthieno[3,2-c]pyridin-4-yl)-N-[(3R)-3-piperidyl]pyridine-3-carboxamide (Example 885)

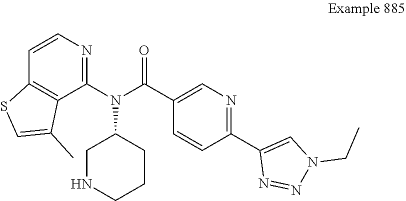 [SMILES: CCn1cc(-c2ccc(C(=O)N(c3nccc4scc(C)c34)[C@@H]3CCCNC3)cn2)nn1]

Example 885 was prepared according to the procedure described for Example 111 by substituting tert-butyl (R)-3-((8-methylisoquinolin-1-yl)amino)piperidine-1-carboxylate with tert-butyl (R)-3-((3-methylthieno[3,2-c]pyridin-4-yl)amino)piperidine-1-carboxylate; 4-(methoxycarbonyl)phenyl) boronic acid with 3-methyl 6-borono-3-pyridinecarboxylate; 4-bromo-1-methyl-1H-1,2,3-triazole with 4-bromo-1-ethyl-1H-1,2,3-triazole, provided the title compound as a light brown solid. LC-MS: [M+H]=448.2. ¹H NMR (400 MHZ, CD₃OD): δ ppm 8.51-8.47 (m, 2H), 8.31 (s, 1H), 8.03 (d, J=5.5 Hz, 1H), 7.86 (s, 2H), 7.40 (d, J=1.0 Hz, 1H), 5.20-5.11 (m, 1H), 4.48 (q, J=7.3 Hz, 2H), 3.99-3.90 (m, 1H), 3.80-3.69 (m, 1H), 3.37 (br d, J=12.5 Hz, 1H), 2.99-2.89 (m, 1H), 2.60-2.47 (m, 3H), 1.97-1.79 (m, 3H), 1.54 (t, J=7.3 Hz, 3H), 1.35-1.23 (m, 1H).

### Synthesis of 2-fluoro-4-(5-methyl-1,3,4-thiadiazol-2-yl)-N-[(3R)-3-piperidyl]-N-(3-vinylthieno[3,2-c]pyridin-4-yl)benzamide (Example 886)

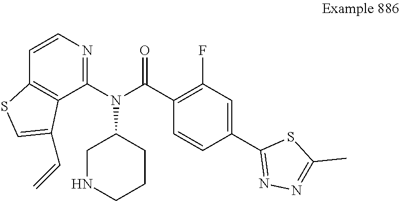 [SMILES: C=Cc1csc2ccnc(N(C(=O)c3ccc(-c4nnc(C)s4)cc3F)[C@@H]3CCCNC3)c12]

Example 886 was prepared according to the procedure described for Example 651 by substituting potassium isopropenyltrifluoroborate with potassium vinyltrifluoroborate, provided the title compound as a white solid. LC-MS: [M+H+]=480.2. ¹H NMR (400 MHZ, DMSO-d₆): δ ppm 8.50-8.28 (m, 1H), 8.23-8.08 (m, 1H), 8.01 (d, J=5.5 Hz, 1H), 7.52 (br d, J=10.3 Hz, 1H), 7.45-7.34 (m, 1H), 7.18-6.95 (m, 2H), 5.97-5.77 (m, 1H), 5.52 (d, J=10.9 Hz, 1H), 4.60-4.35 (m, 1H), 3.46 (br d, J=10.1 Hz, 1H), 2.97 (br t, J=10.9 Hz, 1H), 2.83-2.70 (m, 4H), 2.22 (br d, J=12.1 Hz, 1H), 1.75-1.34 (m, 3H), 0.89-0.73 (m, 1H).

### Synthesis of 2-fluoro-N-(4-isopropenyl-2-pyridyl)-4-(1-methyltriazol-4-yl)-N-[(3R)-3-piperidyl]benzamide (Example 887)

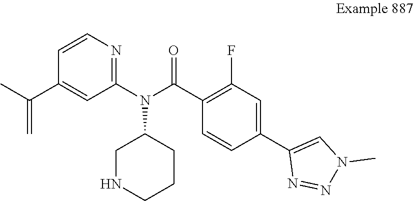 [SMILES: C=C(C)c1ccnc(N(C(=O)c2ccc(-c3cn(C)nn3)cc2F)[C@@H]2CCCNC2)c1]

Example 887 was prepared according to the procedure described for Example 110 by substituting 1H-pyrazol-3-ylboronic acid with 2-isopropenyl-4,4,5,5-tetramethyl-1,3,2-dioxaborolane, provided the title compound as a light brown solid. LC-MS: [M+H]=421.2. ¹H NMR (400 MHZ, CD₃OD): δ ppm 8.41 (d, J=5.4 Hz, 1H), 8.31 (s, 1H), 7.55 (dd, J=1.5, 8.1 Hz, 1H), 7.46-7.39 (m, 2H), 7.37 (dd, J=1.5, 5.4 Hz, 1H), 7.22 (s, 1H), 5.44 (s, 1H), 5.24 (s, 1H), 5.04-4.91 (m, 1H), 4.13 (s, 3H), 3.79-3.70 (m, 1H), 3.58-3.47 (m, 1H), 3.39-3.35 (m, 1H), 2.91 (dt, J=2.9, 12.7 Hz, 1H), 2.13-2.01 (m, 2H), 1.96 (s, 3H), 1.92-1.73 (m, 2H).

### Synthesis of 4-[1-(2-cyanoethyl)triazol-4-yl]-2-fluoro-N-(8-methyl-1-isoquinolyl)-N-[(3R)-3-piperidyl]benzamide (Example 888)

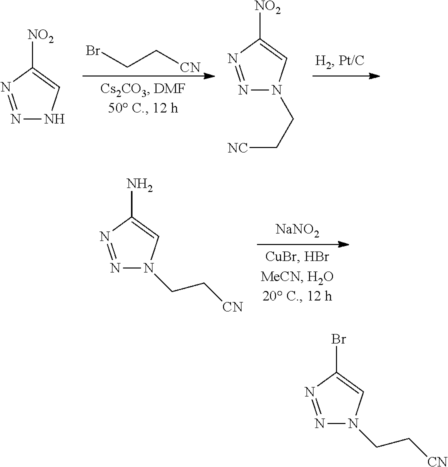 [SMILES: O=[N+]([O-])c1c[nH]nn1 ; N#CCCBr ; N#CCCn1cc([N+](=O)[O-])nn1 ; N#CCCn1cc(N)nn1 ; "NaNO2" ; N#CCCn1cc(Br)nn1]

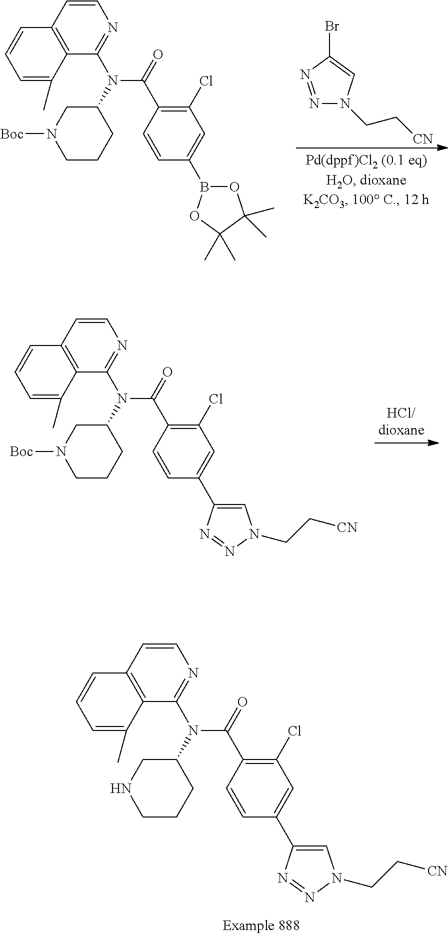 [SMILES: Cc1cccc2ccnc(N(C(=O)c3ccc(B4OC(C)(C)C(C)(C)O4)cc3Cl)[C@@H]3CCCN(C(=O)OC(C)(C)C)C3)c12 ; N#CCCn1cc(Br)nn1 ; Cc1cccc2ccnc(N(C(=O)c3ccc(-c4cn(CCC#N)nn4)cc3Cl)[C@@H]3CCCN(C(=O)OC(C)(C)C)C3)c12 ; Cc1cccc2ccnc(N(C(=O)c3ccc(-c4cn(CCC#N)nn4)cc3Cl)[C@@H]3CCCNC3)c12]

### Step 1:

To a solution of 3-bromopropanenitrile (7.05 g, 52.60 mmol, 4.31 mL) in DMF (40 mL) was added Cs₂CO₃ (12.57 g, 38.58 mmol) and 4-nitro-1H-triazole (4 g, 35.07 mmol) in order at 20° C. under N₂ atmosphere. Then the mixture was stirred at 50° C. for 2 h under N₂ atmosphere. The reaction mixture was filtered and the filtrate was diluted with water (100 mL) and extracted with EtOAc (3×100 mL). The combined organic layer was washed with brine (6×50 mL), dried over Na₂SO₄, filtered and concentrated under reduced pressure to give a residue, which was purified by flash column using 50% EtOAc in petroleum ether to give 3-(4-nitrotriazol-1-yl)propanenitrile (1 g, 15%) as an off white solid.

### Step 2:

To a solution of 3-(4-nitrotriazol-1-yl)propanenitrile (1 g, 5.39 mmol) in THF (10 mL) was flowed through a pipe attached with 1% Pt/C at 40° C. under H₂ (0.5 Mpa) over 3.3 mins. The reaction mixture was concentrated under reduced pressure to give 3-(4-aminotriazol-1-yl) propanenitrile (0.6 g, crude) as a yellow solid. LC-MS: [M+H]=138.2.

### Step 3:

To a solution of 3-(4-aminotriazol-1-yl)propanenitrile (0.4 g, 2.92 mmol) in MeCN (16 mL) and H₂O (4 mL) was added HBr (2.95 g, 14.58 mmol, 1.98 mL, 40% purity) and CuBr (502.08 mg, 3.50 mmol, 106.60 μL) in order at 0° C. under N₂ atmosphere. Then NaNO₂ (301.86 mg, 4.38 mmol) was added to the above mixture at 0° C. Then the mixture was stirred at 20° C. for 12 h under N₂ atmosphere. The mixture was diluted with water (50 mL) and extracted with EtOAc (3×50 mL). The combined organic layer was washed with brine (3×50 mL), dried over Na₂SO₄, filtered and concentrated under reduced pressure to give a residue, which was purified by reverse phase prep-HPLC to give 3-(4-bromotriazol-1-yl) propanenitrile (0.3 g, 46%) as a light yellow solid. LC-MS: [M+H]=201.0.

### Step 4:

To a solution of tert-butyl (3R)-3-[[2-fluoro-4-(4,4,5,5-tetramethyl-1,3,2-dioxaborolan-2-yl)benzoyl]-(8-methyl-1-isoquinolyl)amino]piperidine-1-carboxylate (0.2 g, 339.27 μmol) in dioxane (1.8 mL) and H₂O (0.2 mL) was added 3-(4-bromotriazol-1-yl)propanenitrile (151.56 mg, 678.54 μmol), K₂CO₃ (140.67 mg, 1.02 mmol) and Pd(dppf)Cl₂ (24.82 mg, 33.93 μmol) in order at 20° C. under N₂ atmosphere. Then the mixture was stirred at 100° C. for 1 h under N₂ atmosphere. The reaction mixture was filtrated and the filter liquor diluted with water (5 mL) and extracted with EtOAc (3×5 mL). The combined organic layer was washed with brine (6×5 mL), dried over Na₂SO₄, filtered and concentrated under reduced pressure to give a residue. The residue was purified by prep-TLC using 70% EtOAc in petroleum ether to give tert-butyl (3R)-3-[[4-[1-(2-cyanoethyl)triazol-4-yl]-2-fluoro-benzoyl]-(8-methyl-1-isoquinolyl)amino]-piperidine-1-carboxylate (0.05 g, 22%) as a yellow solid. LC-MS: [M+H]=584.4.

### Step 5:

A solution of tert-butyl (3R)-3-[[4-[1-(2-cyanoethyl)triazol-4-yl]-2-fluoro-benzoyl]-(8-methyl-1-isoquinolyl)amino]piperidine-1-carboxylate (0.05 g, 72.57 μmol) in 4 M HCl/dioxane (0.5 mL) was stirred at 20° C. for 1 h. The reaction mixture was concentrated under reduced pressure to give a residue. The residue was purified by reverse phase prep-HPLC to give 4-[1-(2-cyanoethyl)triazol-4-yl]-2-fluoro-N-(8-methyl-1-isoquinolyl)-N-[(3R)-3-piperidyl]benzamide (0.014 g, 35%) as white solid. LC-MS: [M+H]=484.3. ¹H NMR (400 MHz, CD₃OD): δ ppm 8.43 (d, J=5.5 Hz, 1H), 8.30 (s, 1H), 7.72 (d, J=5.5 Hz, 1H), 7.64 (d, J=7.4 Hz, 1H), 7.49 (q, J=7.5 Hz, 2H), 7.27 (dd, J=1.3, 11.4 Hz, 1H), 7.15 (dd, J=1.3, 8.1 Hz, 1H), 6.87 (t, J=7.6 Hz, 1H), 5.07 (br s, 1H), 4.68 (t, J=6.5 Hz, 2H), 4.00 (br dd, J=2.3, 11.4 Hz, 1H), 3.89-3.78 (m, 1H), 3.42 (br d, J=1.5 Hz, 1H), 3.13 (d, J=6.5 Hz, 2H), 3.05-2.96 (m, 1H), 2.84 (s, 3H), 2.03-1.88 (m, 3H), 1.67-1.51 (m, 1H).

### Synthesis of 4-(1-ethyltriazol-4-yl)-2-fluoro-N-[8-methyl-6-(trideuteriomethoxy)-1-isoquinolyl]-N-[(3R)-3-piperidyl]benzamide (Example 889)

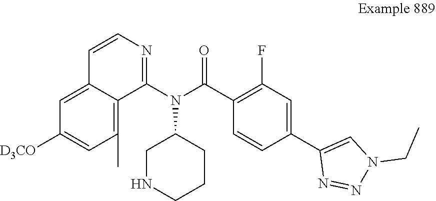

Example 889 was prepared according to the procedure described for Example 127 by substituting tert-butyl (R)-3-((8-methylisoquinolin-1-yl)amino)piperidine-1-carboxylate with tert-butyl (R)-3-((6-(methoxy-d3)-8-methylisoquinolin-1-yl)amino)piperidine-1-carboxylate and 2-bromo-5-methyl-1,3,4-thiadiazole with 4-bromo-1-ethyl-1H-1,2,3-triazole, provided the title compound as a brown solid. LC-MS: [M+H]=492.3. ¹H NMR (400 MHZ, CD₃OD): δ ppm 8.32-8.28 (m, 1H), 8.25 (s, 1H), 7.65-7.58 (m, 1H), 7.33-7.24 (m, 1H), 7.18 (dd, J=1.3, 8.1 Hz, 1H), 7.11-7.05 (m, 1H), 7.03-6.97 (m, 1H), 6.94-6.86 (m, 1H), 5.06-4.97 (m, 1H), 4.41 (q, J=7.3 Hz, 2H), 3.98 (br dd, J=3.1, 12.2 Hz, 1H), 3.84-3.75 (m, 1H), 3.45-3.36 (m, 2H), 3.06-2.95 (m, 1H), 2.83-2.75 (m, 3H), 2.73-2.64 (m, 1H), 2.00-1.85 (m, 3H), 1.64-1.54 (m, 1H), 1.51 (t, J=7.4 Hz, 3H).

### Synthesis of 2-fluoro-N-(8-methyl-1-isoquinolyl)-N-[(3R)-3-piperidyl]-4-[1-(trideuteriomethyl)triazol-4-yl]benzamide (Example 890)

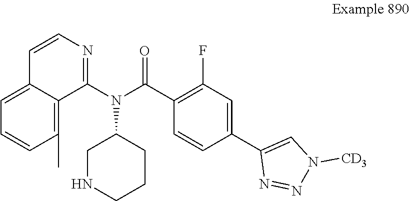 [SMILES: [2H]C([2H])([2H])n1cc(-c2ccc(C(=O)N(c3nccc4cccc(C)c34)[C@@H]3CCCNC3)c(F)c2)nn1]

Example 890 was prepared according to the procedure described for Example 127 by substituting 2-bromo-5-methyl-1,3,4-thiadiazole with 4-bromo-1-(methyl-d3)-1H-1,2,3-triazole, provided the title compound as a white solid. LC-MS: [M+H]=448.3. ¹H NMR (400 MHZ, CD₃OD): δ ppm 8.45-8.41 (m, 1H), 8.15 (s, 1H), 7.73 (d, J=5.5 Hz, 1H), 7.67-7.60 (m, 1H), 7.52-7.45 (m, 2H), 7.28-7.21 (m, 1H), 7.13 (dd, J=1.4, 8.1 Hz, 1H), 6.89-6.83 (m, 1H), 5.11-5.03 (m, 1H), 4.04-3.96 (m, 1H), 3.88-3.78 (m, 1H), 3.42 (br s, 1H), 3.06-2.97 (m, 1H), 2.84 (s, 3H), 1.99-1.88 (m, 3H), 1.65-1.55 (m, 1H).

### Synthesis of 2-fluoro-N-(6-fluoro-8-methyl-1-isoquinolyl)-N-[(3R)-3-piperidyl]-4-[1-(trideuteriomethyl)triazol-4-yl]benzamide (Example 891)

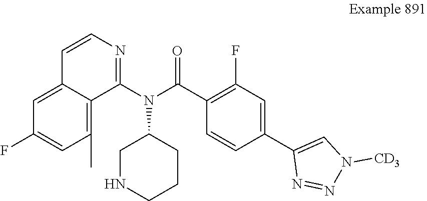 [SMILES: [2H]C([2H])([2H])n1cc(-c2ccc(C(=O)N(c3nccc4cc(F)cc(C)c34)[C@@H]3CCCNC3)c(F)c2)nn1]

Example 891 was prepared according to the procedure described for Example 127 by substituting tert-butyl (R)-3-((8-methylisoquinolin-1-yl)amino)piperidine-1-carboxylate with tert-butyl (R)-3-((6-fluoro-8-methylisoquinolin-1-yl)amino)piperidine-1-carboxylate and 2-bromo-5-methyl-1,3,4-thiadiazole with 4-bromo-1-(methyl-d3)-1H-1,2,3-triazole, provided the title compound as off white solid. LC-MS: [M+H]=466.4. ¹H NMR (400 MHZ, CD₃OD): δ ppm 8.37-8.45 (m, 1H), 8.17 (s, 1H), 7.64-7.73 (m, 1H), 7.29-7.36 (m, 2H), 7.21-7.29 (m, 1H), 7.17 (dd, J=8.13, 1.50 Hz, 1H), 6.82-6.90 (m, 1H), 4.98-5.13 (m, 1H), 3.95-4.03 (m, 1H), 3.76-3.89 (m, 1H), 3.41 (br d, J=12.76 Hz, 2H), 2.95-3.08 (m, 1H), 2.82-2.90 (m, 4H), 2.62-2.79 (m, 1H), 1.85-2.05 (m, 3H), 1.54-1.69 (m, 1H).

### Synthesis of N-(6-chloro-8-methyl-1-isoquinolyl)-N-[(3R)-3-piperidyl]-4-[1-(trideuteriomethyl)triazol-4-yl] benzamide (Example 892)

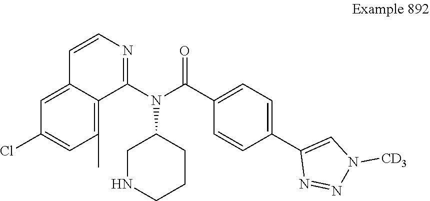 [SMILES: [2H]C([2H])([2H])n1cc(-c2ccc(C(=O)N(c3nccc4cc(Cl)cc(C)c34)[C@@H]3CCCNC3)cc2)nn1]

Example 892 was prepared according to the procedure described for Example 111 by substituting tert-butyl (R)-3-((8-methylisoquinolin-1-yl)amino)piperidine-1-carboxylate with tert-butyl (R)-3-((6-chloro-8-methylisoquinolin-1-yl)amino)piperidine-1-carboxylate and 4-bromo-1-methyl-1H-1,2,3-triazole with 4-bromo-1-(methyl-d3)-1H-1,2,3-triazole, provided the title compound as a white solid. LC-MS: [M+H+]=464.2. 1H NMR (400 MHZ, CD₃OD): δ ppm 8.55-8.48 (m, 1H), 8.14 (s, 1H), 7.73-7.68 (m, 2H), 7.45 (d, J=8.3 Hz, 2H), 7.37 (s, 1H), 7.11 (br d, J=8.3 Hz, 2H), 4.83-4.62 (m, 1H), 3.68 (br d, J=11.1 Hz, 1H), 3.39 (br t, J=11.3 Hz, 1H), 3.10-2.97 (m, 1H), 2.82-2.73 (m, 3H), 2.64-2.41 (m, 2H), 1.96-1.64 (m, 3H), 1.43-1.29 (m, 1H).

### Synthesis of N-(6-chloro-8-methyl-1-isoquinolyl)-2-fluoro-N-[(3R)-3-piperidyl]-4-[1-(trideuteriomethyl)triazol-4-yl]benzamide (Example 893)

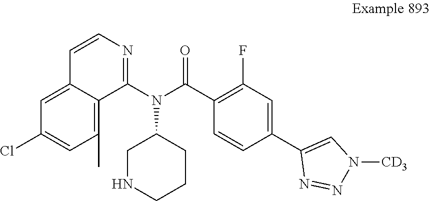 [SMILES: [2H]C([2H])([2H])n1cc(-c2ccc(C(=O)N(c3nccc4cc(Cl)cc(C)c34)[C@@H]3CCCNC3)c(F)c2)nn1]

Example 893 was prepared according to the procedure described for Example 127 by substituting tert-butyl (R)-3-((8-methylisoquinolin-1-yl)amino)piperidine-1-carboxylate with tert-butyl (R)-3-((6-chloro-8-methylisoquinolin-1-yl)amino)piperidine-1-carboxylate and 2-bromo-5-methyl-1,3,4-thiadiazole with 4-bromo-1-(methyl-d3)-1H-1,2,3-triazole, provided the title compound as a yellow solid. LC-MS: [M+H+]=482.2. ¹H NMR (400 MHZ, CD₃OD): δ ppm 8.53-8.41 (m, 1H), 8.18 (s, 1H), 7.72-7.62 (m, 2H), 7.49-7.42 (m, 1H), 7.30-7.15 (m, 2H), 6.90-6.83 (m, 1H), 5.12-5.00 (m, 1H), 4.02-3.94 (m, 1H), 3.87-3.78 (m, 1H), 3.46-3.37 (m, 1H), 3.07-2.95 (m, 1H), 2.86-2.81 (m, 3H), 2.22-1.86 (m, 3H), 1.66-1.53 (m, 1H).

### Synthesis of N-(3-methylthieno[3,2-c]pyridin-4-yl)-N-[(3R)-3-piperidyl]-4-[5-(trideuteriomethyl)-1,3,4-thiadiazol-2-yl]benzamide (Example 894)

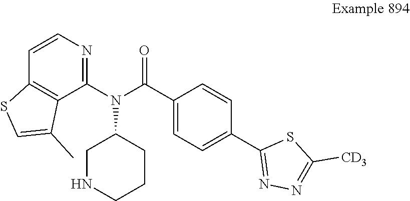 [SMILES: [2H]C([2H])([2H])c1nnc(-c2ccc(C(=O)N(c3nccc4scc(C)c34)[C@@H]3CCCNC3)cc2)s1]

Example 894 was prepared according to the procedure described for Example 895 by substituting tert-butyl (R)-3-((6-chloro-8-methylisoquinolin-1-yl)amino)piperidine-1-carboxylate with tert-butyl (R)-3-((3-methylthieno[3,2-c]pyridin-4-yl)amino)piperidine-1-carboxylate; methyl 5-chlorocarbonylpyridine-2-carboxylate with methyl 4-(chlorocarbonyl)benzoate; 2-methoxyacetohydrazide with acetohydrazide-2,2,2-d3, provided the title compound as a white solid. LC-MS: [M+H+]=453.2. ¹H NMR (400 MHZ, CD₃OD) δ ppm 8.49-8.40 (m, 1H), 8.00 (d, J=5.5 Hz, 1H), 7.65 (d, J=8.4 Hz, 2H), 7.44-7.27 (m, 3H), 5.14 (tt, J=3.8, 11.6 Hz, 1H), 3.93 (br d, J=11.0 Hz, 1H), 3.75 (t, J=11.5 Hz, 1H), 3.37 (br d, J=12.4 Hz, 1H), 3.02-2.87 (m, 1H), 2.62-2.47 (m, 3H), 1.98-1.85 (m, 2H), 1.80 (br d, J=12.8 Hz, 1H), 1.39-1.23 (m, 1H).

### Synthesis of N-(6-chloro-8-methyl-1-isoquinolyl)-6-[5-(methoxymethyl)-1,3,4-thiadiazol-2-yl]-N-[(3R)-3-piperidyl]pyridine-3-carboxamide (Example 895)

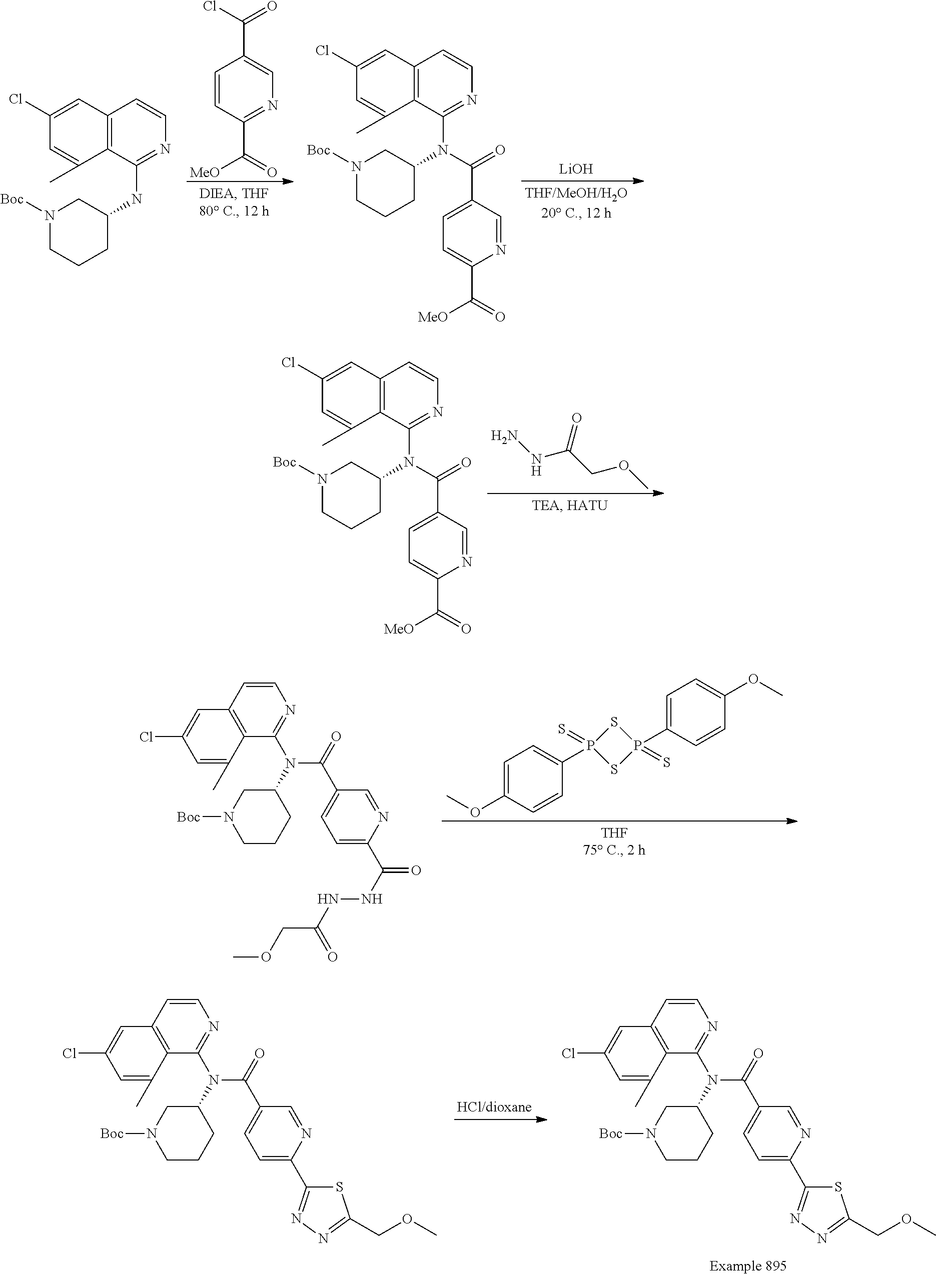

### Step 1:

To a solution of tert-butyl (3R)-3-[(6-chloro-8-methyl-1-isoquinolyl)amino]piperidine-1-carboxylate (1.5 g, 3.99 mmol) in THF (15 mL) was added DIEA (4.13 g, 31.92 mmol, 5.56 mL) and methyl 5-chlorocarbonylpyridine-2-carboxylate (1.77 g, 7.98 mmol) at 20° C. under N₂ atmosphere. The mixture was purged with N₂ for three times. The mixture was stirred at 80° C. for 12 h. The reaction mixture was filtered and concentrated. The crude product was purified by flash column using EtOAc to give methyl 5-[[(3R)-1-tert-butoxycarbonyl-3-piperidyl]-(6-chloro-8-methyl-1-isoquinolyl)carbamoyl]pyridine-2-carboxylate (1.9 g, 79%) as a yellow solid.

### Step 2:

To a solution of methyl 5-[[(3R)-1-tert-butoxycarbonyl-3-piperidyl]-(6-chloro-8-methyl-1-isoquinolyl)carbamoyl]pyridine-2-carboxylate (1.9 g, 3.17 mmol) in THF (10 mL), MeOH (10 mL) and H₂O (10 mL) was added LiOH (189.93 mg, 7.93 mmol) at 20° C. The mixture was stirred at 20° C. for 12 h. The reaction mixture was concentrated under reduced pressure. The residue was diluted with water (20 mL) and extracted with EtOAc (2×20 mL). The aqueous phase was adjusted pH to 7 with 1 N HCl and extracted with EtOAc (5×20 mL). The combined organic layer was concentrated under reduced pressure to give 5-[[(3R)-1-tert-butoxycarbonyl-3-piperidyl]-(6-chloro-8-methyl-1-isoquinolyl)carbamoyl]pyridine-2-carboxylic acid (1.6 g, 86%) as a yellow solid. LC-MS: [M+H]=469.2.

### Step 3:

To a solution of 5-[[(3R)-1-tert-butoxycarbonyl-3-piperidyl]-(6-chloro-8-methyl-1-isoquinolyl)carbamoyl]pyridine-2-carboxylic acid (0.9 g, 1.54 mmol) in THF (9 mL) was added TEA (468.37 mg, 4.63 mmol, 644.24 μL) and HATU (1.17 g, 3.09 mmol) and 2-methoxyacetohydrazide (321.25 mg, 3.09 mmol) at 20° C. under N₂ atmosphere. The mixture was charged with N₂ for three times. The mixture was stirred at 20° C. for 12 h. The reaction mixture was quenched with water (20 mL) at 20° C., and then extracted with EtOAc (4×50 mL). The combined organic layer was washed with brine (100 mL), dried over Na₂SO₄, filtered and concentrated under reduced pressure to give tert-butyl (3R)-3-[(6-chloro-8-methyl-1-isoquinolyl)-[6-[[(2-methoxyacetyl)amino]carbamoyl]pyridine-3-carbonyl]amino]piperidine-1-carboxylate (1.5 g, 95%) as a yellow solid. LC-MS: [M+H]=611.3.

### Step 4:

To a solution of tert-butyl (3R)-3-[(6-chloro-8-methyl-1-isoquinolyl)-[6-[[(2-methoxyacetyl)amino]carbamoyl]pyridine-3-carbonyl]amino]piperidine-1-carboxylate (0.95 g, 932.76 μmol) in THF (9.5 mL) was added 2,4-bis(4-methoxyphenyl)-2,4-dithioxo-1,3,2,4dithiadiphosphetane (754.54 mg, 1.87 mmol) at 20° C. under N₂ atmosphere. The mixture was stirred at 75° C. for 2 h. The reaction mixture was quenched with water (20 mL) at 20° C., and then extracted with EtOAc (5×30 mL). The combined organic layer was washed with brine (100 mL), dried over Na₂SO₄, filtered and concentrated. The crude product was purified by reverse-phase prep-HPLC to give tert-butyl (3R)-3-[(6-chloro-8-methyl-1-isoquinolyl)-[6-[5-(methoxymethyl)-1,3,4-thiadiazol-2-yl]pyridine-3-carbonyl]amino]piperidine-1-carboxylate (300 mg, 50%) as an off white solid. LC-MS: [M+H]=609.3.

### Step 5:

To a solution of tert-butyl (3R)-3-[(6-chloro-8-methyl-1-isoquinolyl)-[6-[5-(methoxymethyl)-1,3,4-thiadiazol-2-yl]pyridine-3-carbonyl]amino]piperidine-1-carboxylate (0.2 g, 311.92 μmol in dioxane (1 mL) was added 4 M HCl/dioxane (200 μL) at 20° C. The mixture was stirred at 20° C. for 1 h. The reaction mixture was concentrated under reduced pressure to give the crude product. The crude product was purified by reverse phase prep-HPLC to give N-(6-chloro-8-methyl-1-isoquinolyl)-6-[5-(methoxymethyl)-1,3,4-thiadiazol-2-yl]-N-[(3R)-3-piperidyl]pyridine-3-carboxamide (0.1 g, 56%) as an off white solid. LC-MS: [M+H]=509.2. ¹H NMR (400 MHZ, CD₃OD): δ=1.43-1.59 (m, 1H) 1.82-2.03 (m, 3H) 2.73-2.84 (m, 3H) 2.93-3.06 (m, 1H) 3.37-3.50 (m, 4H) 3.76-3.86 (m, 1H) 3.98 (br d, J=11.76 Hz, 1H) 4.82 (s, 2H) 5.03-5.16 (m, 1H) 7.44 (s, 1H) 7.55-7.62 (m, 1H) 7.76-7.84 (m, 2H) 7.88-7.94 (m, 1H) 8.22-8.26 (m, 1H) 8.51-8.61 (m, 1H).

### Synthesis of N-(6,8-dimethyl-1-isoquinolyl)-6-[5-(methoxymethyl)-1,3,4-thiadiazol-2-yl]-N-[(3R)-3-piperidyl] pyridine-3-carboxamide (Example 896)

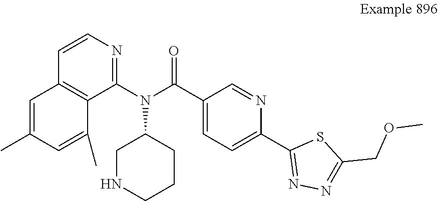

Example 896 was prepared according to the procedure described for Example 895 by substituting tert-butyl (R)-3-((6-chloro-8-methylisoquinolin-1-yl)amino)piperidine-1-carboxylate with tert-butyl (R)-3-((6,8-dimethylisoquinolin-1-yl)amino)piperidine-1-carboxylate, provided the title compound as off white solid. LC-MS: [M+H]=489.3. ¹H NMR (400 MHZ, CD₃OD): δ=1.37-1.54 (m, 1H) 1.78-2.20 (m, 3H) 2.36 (s, 3H) 2.70-2.82 (m, 3H) 2.92-3.06 (m, 1H) 3.35-3.51 (m, 4H) 3.75-3.84 (m, 1H) 3.98 (br d, J=11.63 Hz, 1H) 4.82 (s, 2H) 5.08 (tt, J=11.41, 3.72 Hz, 1H) 7.24-7.36 (m, 1H) 7.47-7.53 (m, 1H) 7.56-7.62 (m, 1H) 7.70-7.78 (m, 1H) 7.85-7.91 (m, 1H) 8.23-8.28 (m, 1H) 8.42-8.51 (m, 1H).

### Synthesis of N-(5, 8-dimethyl-1-isoquinolyl)-6-(5-methyl-1, 3, 4-thiadiazol-2-yl)-N-[(3R)-3-piperidyl] pyridine-3-carboxamide (Example 897)

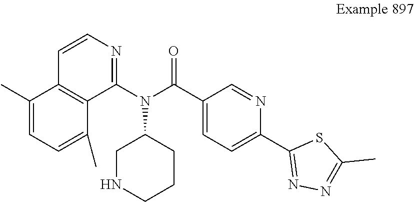

Example 897 was prepared according to the procedure described for Example 895 by substituting tert-butyl (R)-3-((6-chloro-8-methylisoquinolin-1-yl)amino)piperidine-1-carboxylate with tert-butyl (R)-3-((5,8-dimethylisoquinolin-1-yl)amino)piperidine-1-carboxylate and 2-methoxyacetohydrazide with acetohydrazide, provided the title compound as an off white solid. LC-MS: [M+H]=459.2. ¹H NMR (400 MHZ, CD₃OD): δ ppm 1.42-1.57 (m, 1H) 1.81-2.22 (m, 3H) 2.50-2.60 (m, 3H) 2.94-3.09 (m, 1H) 3.43 (br s, 1H) 3.77-3.88 (m, 1H) 3.94-4.10 (m, 1H) 5.09 (br d, J=2.88 Hz, 1H) 7.29-7.43 (m, 2H) 7.56 (dd, J=8.25, 2.00 Hz, 1H) 7.79-7.89 (m, 1H) 7.92-8.08 (m, 1H) 8.17-8.28 (m, 1H) 8.52-8.65 (m, 1H).

### Synthesis of N-(5-chloro-8-methyl-1-isoquinolyl)-6-(5-methyl-1, 3, 4-thiadiazol-2-yl)-N-[(3R)-3-piperidyl] pyridine-3-carboxamide (Example 898)

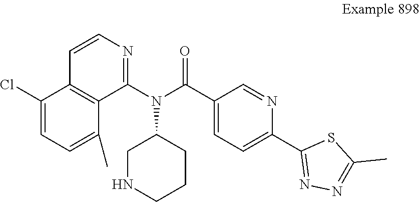 [SMILES: Cc1nnc(-c2ccc(C(=O)N(c3nccc4c(Cl)ccc(C)c34)[C@@H]3CCCNC3)cn2)s1]

Example 898 was prepared according to the procedure described for Example 895 by substituting tert-butyl (R)-3-((6-chloro-8-methylisoquinolin-1-yl)amino)piperidine-1-carboxylate with tert-butyl (R)-3-((5-chloro-8-methylisoquinolin-1-yl)amino)piperidine-1-carboxylate and 2-methoxyacetohydrazide with acetohydrazide, provided the title compound as off white solid. LC-MS: [M+H]=479.2. ¹H NMR (400 MHZ, CD₃OD): δ ppm 1.44-1.64 (m, 1H) 1.80-2.22 (m, 3H) 2.73-2.83 (m, 6H) 2.93-3.07 (m, 1H) 3.39-3.44 (m, 1H) 3.77-3.86 (m, 1H) 3.94-4.04 (m, 1H) 5.06-5.16 (m, 1H) 7.39 (br d, J=7.88 Hz, 1H) 7.49-7.56 (m, 1H) 7.62-7.68 (m, 1H) 7.83-7.88 (m, 1H) 8.14-8.26 (m, 2H) 8.60-8.74 (m, 1H).

### Synthesis of 2-fluoro-N-(8-methyl-1-isoquinolyl)-N-[(3R)-3-piperidyl]-4-[5-(trideuteriomethyl)-1,3,4-thiadiazol-2-yl]benzamide (Example 899)

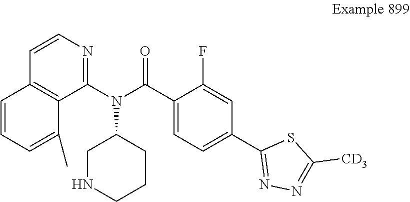 [SMILES: [2H]C([2H])([2H])c1nnc(-c2ccc(C(=O)N(c3nccc4cccc(C)c34)[C@@H]3CCCNC3)c(F)c2)s1]

Example 899 was prepared according to the procedure described for Example 895 by substituting tert-butyl (R)-3-((6-chloro-8-methylisoquinolin-1-yl)amino)piperidine-1-carboxylate with tert-butyl (R)-3-((8-methylisoquinolin-1-yl)amino)piperidine-1-carboxylate and 2-methoxyacetohydrazide with acetohydrazide-2,2,2-d3, provided the title compound as an off white solid. LC-MS: [M+H]=465.3. ¹H NMR (400 MHZ, CD₃OD): δ ppm 1.46-1.65 (m, 1H) 1.86-2.04 (m, 3H) 2.72 (br d, J=3.25 Hz, 1H) 2.83-2.90 (m, 3H) 2.95-3.07 (m, 1H) 3.41 (br d, J=12.51 Hz, 1H) 3.76-3.89 (m, 1H) 4.02 (br dd, J=11.76, 3.25 Hz, 1H) 5.00-5.14 (m, 1H) 6.94-7.04 (m, 1H) 7.26 (dd, J=8.13, 1.38 Hz, 1H) 7.41 (dd, J=10.69, 1.44 Hz, 1H) 7.46-7.55 (m, 2H) 7.61-7.69 (m, 1H) 7.74 (d, J=5.50 Hz, 1H) 8.37-8.48 (m, 1H).

### Synthesis of 6-(5-ethyl-1,3,4-thiadiazol-2-yl)-N-(8-methyl-1-isoquinolyl)-N-[(3R)-3-piperidyl] pyridine-3-carboxamide (Example 900)

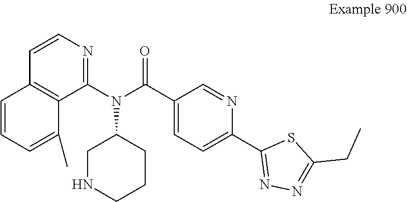 [SMILES: CCc1nnc(-c2ccc(C(=O)N(c3nccc4cccc(C)c34)[C@@H]3CCCNC3)cn2)s1]

Example 900 was prepared according to the procedure described for Example 895 by substituting tert-butyl (R)-3-((6-chloro-8-methylisoquinolin-1-yl)amino)piperidine-1-carboxylate with tert-butyl (R)-3-((8-methylisoquinolin-1-yl)amino)piperidine-1-carboxylate and 2-methoxyacetohydrazide with propionohydrazide, provided the title compound as a yellow solid. LC-MS: [M+H⁺]=459.2. ¹H NMR (400 MHZ, CD₃OD): δ ppm 8.56-8.48 (m, 1H), 8.24-8.19 (m, 1H), 7.88-7.81 (m, 2H), 7.74-7.68 (m, 1H), 7.58-7.41 (m, 3H), 5.15-5.05 (m, 1H), 3.99 (td, J=1.7, 12.0 Hz, 1H), 3.86-3.78 (m, 1H), 3.73-3.46 (m, 1H), 3.40 (br d, J=12.4 Hz, 1H), 3.15-3.08 (m, 2H), 3.06-2.94 (m, 1H), 2.82 (s, 1H), 2.76 (s, 2H), 2.03-1.83 (m, 3H), 1.57-1.44 (m, 1H), 1.39 (t, J=7.5 Hz, 3H).

### Synthesis of N-(6-methoxy-8-methyl-1-isoquinolyl)-6-(5-methyl-1,3,4-thiadiazol-2-yl)-N-[(3R)-3-piperidyl]pyridine-3-carboxamide (Example 901)

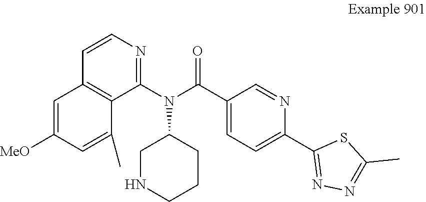 [SMILES: COc1cc(C)c2c(N(C(=O)c3ccc(-c4nnc(C)s4)nc3)[C@@H]3CCCNC3)nccc2c1]

Example 901 was prepared according to the procedure described for Example 895 by substituting tert-butyl (R)-3-((6-chloro-8-methylisoquinolin-1-yl)amino)piperidine-1-carboxylate with tert-butyl (R)-3-((6-methoxy-8-methylisoquinolin-1-yl)amino)piperidine-1-carboxylate and 2-methoxyacetohydrazide with acetohydrazide, provided the compound as a light pink solid. LC-MS: [M+H⁺]=475.3. ¹H NMR (400 MHZ, CD₃OD): δ=8.46-8.36 (m, 1H), 8.28 (d, J=1.6 Hz, 1H), 7.88 (d, J=8.3 Hz, 1H), 7.82-7.72 (m, 1H), 7.61 (dd, J=2.3, 8.3 Hz, 1H), 7.15-7.04 (m, 2H), 5.11-5.00 (m, 1H), 4.02-3.93 (m, 1H), 3.83 (s, 3H), 3.54-3.35 (m, 2H), 3.08-2.92 (m, 1H), 2.80-2.63 (m, 6H), 2.24-1.79 (m, 3H), 1.56-1.39 (m, 1H).

### Synthesis of N-(5-methoxy-8-methyl-1-isoquinolyl)-6-(5-methyl-1, 3, 4-thiadiazol-2-yl)-N-[(3R)-3-piperidyl] pyridine-3-carboxamide (Example 902)

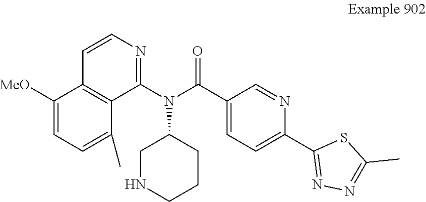 [SMILES: COc1ccc(C)c2c(N(C(=O)c3ccc(-c4nnc(C)s4)nc3)[C@@H]3CCCNC3)nccc12]

Example 902 was prepared according to the procedure described for Example 895 by substituting tert-butyl (R)-3-((6-chloro-8-methylisoquinolin-1-yl)amino)piperidine-1-carboxylate with tert-butyl (R)-3-((5-methoxy-8-methylisoquinolin-1-yl)amino)piperidine-1-carboxylate and 2-methoxyacetohydrazide with acetohydrazide, provided the title compound as an off white solid. LC-MS: [M+H]=475.3. ¹H NMR (400 MHZ, CD₃OD): δ ppm 1.37-1.52 (m, 1H) 1.80-2.20 (m, 3H) 2.64-2.70 (m, 3H) 2.75 (s, 3H) 2.92-3.06 (m, 1H) 3.40 (br s, 1H) 3.73-3.83 (m, 1H) 3.89-3.93 (m, 3H) 3.93-4.02 (m, 1H) 5.03-5.10 (m, 1H) 6.96 (d, J=8.13 Hz, 1H) 7.31-7.39 (m, 1H) 7.52-7.58 (m, 1H) 7.82-7.88 (m, 1H) 8.15-8.23 (m, 2H) 8.48-8.56 (m, 1H).

### Synthesis of N-(6,8-dimethyl-1-isoquinolyl)-6-[5-(2-methoxyethyl)-1,3,4-thiadiazol-2-yl]-N-[(3R)-3-piperidyl]pyridine-3-carboxamide (Example 903)

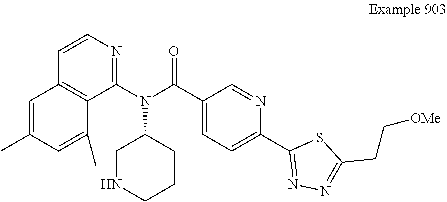 [SMILES: COCCc1nnc(-c2ccc(C(=O)N(c3nccc4cc(C)cc(C)c34)[C@@H]3CCCNC3)cn2)s1]

Example 903 was prepared according to the procedure described for Example 895 by substituting tert-butyl (R)-3-((6-chloro-8-methylisoquinolin-1-yl)amino)piperidine-1-carboxylate with tert-butyl (R)-3-((6,8-dimethylisoquinolin-1-yl)amino)piperidine-1-carboxylate and 2-methoxyacetohydrazide with 3-methoxypropanehydrazide, provided the title compound as an off white solid. LC-MS: [M+H]=503.2. ¹H NMR (400 MHZ, CD₃OD): δ ppm 1.37-1.55 (m, 1H) 1.77-2.01 (m, 3H) 2.36 (s, 3H) 2.62-2.84 (m, 3H) 2.94-3.06 (m, 1H) 3.34-3.58 (m, 5H) 3.70 (t, J=5.88 Hz, 2H) 3.75-3.84 (m, 1H) 3.92-4.04 (m, 1H) 5.01-5.17 (m, 1H) 7.25-7.34 (m, 1H) 7.47-7.53 (m, 1H) 7.55-7.61 (m, 1H) 7.71-7.78 (m, 1H) 7.82-7.89 (m, 1H) 8.24 (br d, J=1.50 Hz, 1H) 8.42-8.51 (m, 1H).

### Synthesis of N-(6,8-dimethyl-1-isoquinolyl)-6-[5-(3-methoxypropyl)-1,3,4-thiadiazol-2-yl]-N-[(3R)-3-piperidyl]pyridine-3-carboxamide (Example 904)

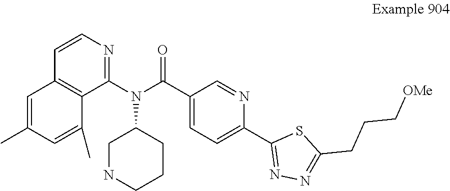 [SMILES: COCCCc1nnc(-c2ccc(C(=O)N(c3nccc4cc(C)cc(C)c34)[C@@H]3CCCNC3)cn2)s1]

Example 904 was prepared according to the procedure described for Example 895 by substituting tert-butyl (R)-3-((6-chloro-8-methylisoquinolin-1-yl)amino)piperidine-1-carboxylate with tert-butyl (R)-3-((6,8-dimethylisoquinolin-1-yl)amino)piperidine-1-carboxylate and 2-methoxyacetohydrazide with 4-methoxybutanehydrazide, provided the title compound as an off white solid. LC-MS: [M+H]=517.3. ¹H NMR (400 MHZ, CD₃OD): δ ppm 1.36-1.54 (m, 1H) 1.79-1.98 (m, 3H) 2.03 (quin, J=6.79 Hz, 2H) 2.36 (s, 3H) 2.63-2.75 (m, 3H) 2.79 (s, 1H) 2.91-3.04 (m, 1H) 3.17 (t, J=7.57 Hz, 2H) 3.38 (br s, 3H) 3.44 (t, J=6.00 Hz, 2H) 3.72-3.87 (m, 1H) 3.98 (br d, J=11.01 Hz, 1H) 5.01-5.17 (m, 1H) 7.27-7.34 (m, 1H) 7.46-7.54 (m, 1H) 7.59 (dd, J=8.19, 1.94 Hz, 1H) 7.69-7.79 (m, 1H) 7.86 (br d, J=8.38 Hz, 1H) 8.25 (br s, 1H) 8.42-8.52 (m, 1H).

### Synthesis of N-(6-methoxy-8-methyl-1-isoquinolyl)-N-[(3R)-3-piperidyl]-6-[5-(trideuteriomethyl)-1,3,4-thiadiazol-2-yl]pyridine-3-carboxamide (Example 905)

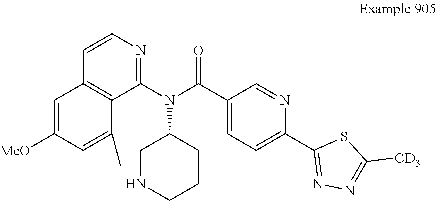 [SMILES: [2H]C([2H])([2H])c1nnc(-c2ccc(C(=O)N(c3nccc4cc(OC)cc(C)c34)[C@@H]3CCCNC3)cn2)s1]

Example 905 was prepared according to the procedure described for Example 895 by substituting tert-butyl (R)-3-((6-chloro-8-methylisoquinolin-1-yl)amino)piperidine-1-carboxylate with tert-butyl (R)-3-((6-methoxy-8-methylisoquinolin-1-yl)amino)piperidine-1-carboxylate and 2-methoxyacetohydrazide with acetohydrazide-2,2,2-d3, provided the title compound as yellow solid. LC-MS: [M+H⁺]=478.3. ¹H NMR (400 MHZ, CD₃OD): δ ppm 8.43 (d, J=5.6 Hz, 1H), 8.28 (d, J=2.0 Hz, 1H), 7.88 (d, J=8.4 Hz, 1H), 7.78 (d, J=5.6 Hz, 1H), 7.61 (dd, J=2.0, 8.3 Hz, 1H), 7.13-7.05 (m, 2H), 5.11-5.00 (m, 1H), 3.97 (br d, J=12.0 Hz, 1H), 3.83 (s, 3H), 3.81-3.74 (m, 1H), 3.39 (br d, J=13.1 Hz, 1H), 3.05-2.95 (m, 1H), 2.77-2.69 (m, 3H), 2.00-1.80 (m, 3H), 1.56-1.41 (m, 1H).

### Synthesis of N-(6,8-dimethyl-1-isoquinolyl)-2-fluoro-N-[(3R)-3-piperidyl]-4-[5-(trideuteriomethyl)-1,3,4-thiadiazol-2-yl]benzamide (Example 906)

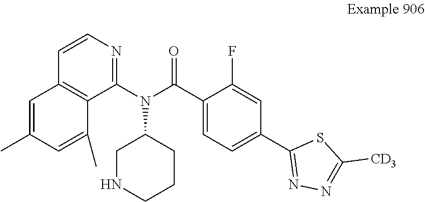 [SMILES: [2H]C([2H])([2H])c1nnc(-c2ccc(C(=O)N(c3nccc4cc(C)cc(C)c34)[C@@H]3CCCNC3)c(F)c2)s1]

Example 906 was prepared according to the procedure described for Example 895 by substituting tert-butyl (R)-3-((6-chloro-8-methylisoquinolin-1-yl)amino)piperidine-1-carboxylate with tert-butyl (R)-3-((6,8-dimethylisoquinolin-1-yl)amino)piperidine-1-carboxylate; methyl 5-chlorocarbonylpyridine-2-carboxylate with methyl 4-(chlorocarbonyl)-3-fluorobenzoate; and 2-methoxyacetohydrazide with acetohydrazide-2,2,2-d3, provided the title compound as a white solid. LC-MS: [M+H⁺]=479.3.

¹H NMR (400 MHZ, CD₃OD): δ ppm 8.35-8.28 (m, 1H), 7.57 (d, J=5.5 Hz, 1H), 7.45-7.37 (m, 2H), 7.34 (s, 1H), 7.25 (br d, J=8.0 Hz, 1H), 7.07-6.97 (m, 1H), 4.73-4.56 (m, 1H), 3.73 (br d, J=11.9 Hz, 1H), 3.03-2.93 (m, 1H), 2.93-2.65 (m, 4H), 2.56-2.44 (m, 1H), 2.42-2.30 (m, 4H), 1.81-1.59 (m, 3H), 1.35-1.22 (m, 1H).

### Synthesis of N-(6,8-dimethyl-1-isoquinolyl)-N-[(3R)-3-piperidyl]-4-[5-(trideuteriomethyl)-1,3,4-thiadiazol-2-yl]benzamide (Example 907)

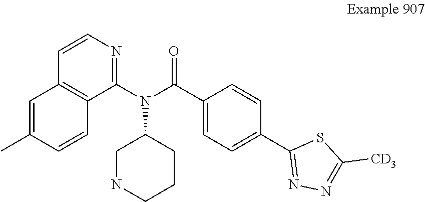 [SMILES: [2H]C([2H])([2H])c1nnc(-c2ccc(C(=O)N(c3nccc4cc(C)ccc34)[C@@H]3CCCNC3)cc2)s1]

Example 907 was prepared according to the procedure described for Example 895 by substituting tert-butyl (R)-3-((6-chloro-8-methylisoquinolin-1-yl)amino)piperidine-1-carboxylate with tert-butyl (R)-3-((6,8-dimethylisoquinolin-1-yl)amino)piperidine-1-carboxylate; methyl 5-chlorocarbonylpyridine-2-carboxylate with methyl 4-(chlorocarbonyl)benzoate; and 2-methoxyacetohydrazide with acetohydrazide-2,2,2-d3, provided the title compound as a white solid. LC-MS: [M+H+]=461.3. ¹H NMR (400 MHZ, CD₃OD): δ ppm 8.43-8.36 (m, 1H), 7.67-7.62 (m, 1H), 7.54 (d, J=8.4 Hz, 2H), 7.43 (s, 1H), 7.29-7.17 (m, 3H), 4.77-4.58 (m, 1H), 3.68 (br d, J=12.3 Hz, 1H), 3.38-3.32 (m, 1H), 3.05-2.94 (m, 1H), 2.86-2.59 (m, 4H), 2.55-2.44 (m, 1H), 2.35 (s, 3H), 1.79-1.60 (m, 3H), 1.34-1.17 (m, 1H).

### Synthesis of N-(6-methoxy-8-methyl-1-isoquinolyl)-N-[(3R)-3-piperidyl]-4-[5-(trideuteriomethyl)-1,3,4-thiadiazol-2-yl]benzamide (Example 908)

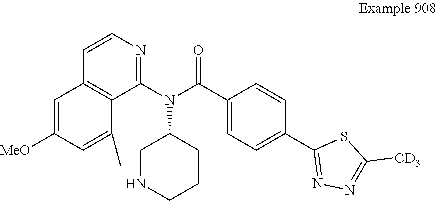 [SMILES: [2H]C([2H])([2H])c1nnc(-c2ccc(C(=O)N(c3nccc4cc(OC)cc(C)c34)[C@@H]3CCCNC3)cc2)s1]

Example 908 was prepared according to the procedure described for Example 895 by substituting tert-butyl (R)-3-((6-chloro-8-methylisoquinolin-1-yl)amino)piperidine-1-carboxylate with tert-butyl (R)-3-((6-methoxy-8-methylisoquinolin-1-yl)amino)piperidine-1-carboxylate; methyl 5-chlorocarbonylpyridine-2-carboxylate with methyl 4-(chlorocarbonyl)benzoate; and 2-methoxyacetohydrazide with acetohydrazide-2,2,2-d3, provided the title compound as a yellow solid. LC-MS: [M+H⁺]=477.2. ¹H NMR (400 MHZ, CD₃OD): δ ppm 8.39-8.31 (m, 1H), 7.68-7.61 (m, 1H), 7.55 (d, J=8.5 Hz, 2H), 7.28-7.17 (m, 2H), 7.04-6.95 (m, 2H), 4.75-4.68 (m, 1H), 3.80 (s, 3H), 3.68 (br d, J=11.1 Hz, 1H), 3.39-3.32 (m, 1H), 3.04-2.95 (m, 1H), 2.75-2.67 (m, 3H), 2.58-2.44 (m, 1H), 1.94-1.62 (m, 3H), 1.37-1.21 (m, 1H).

### Synthesis of 6-(5-methyl-1,3,4-thiadiazol-2-yl)-N-[(3R)-3-piperidyl]-N-[6-(trideuteriomethoxy)-1-isoquinolyl]pyridine-3-carboxamide (Example 909)

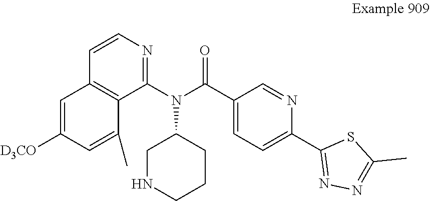

Example 909 was prepared according to the procedure described for Example 895 by substituting tert-butyl (R)-3-((6-chloro-8-methylisoquinolin-1-yl)amino)piperidine-1-carboxylate with tert-butyl (R)-3-((6-(methoxy-d3)-8-methylisoquinolin-1-yl)amino)piperidine-1-carboxylate and 2-methoxyacetohydrazide with acetohydrazide, provided the title compound as a brown solid. LC-MS: [M+H]=478.3. ¹H NMR (400 MHZ, METHANOL-d4): δ ppm 8.44-8.36 (m, 1H), 8.28-8.24 (m, 1H), 7.91-7.84 (m, 1H), 7.78-7.69 (m, 1H), 7.62-7.55 (m, 1H), 7.12-7.02 (m, 2H), 5.11-4.99 (m, 1H), 4.01-3.92 (m, 1H), 3.83-3.73 (m, 1H), 3.39 (br d, J=12.6 Hz, 2H), 3.18 (br d, J=11.8 Hz, 1H), 3.04-2.97 (m, 1H), 2.76 (s, 3H), 2.69 (s, 3H), 1.99-1.80 (m, 3H), 1.54-1.43 (m, 1H).

### Synthesis of (R)-6-(5-(2-methoxyethyl)-1,3,4-thiadiazol-2-yl)-N-(8-methylisoquinolin-1-yl)-N-(piperidin-3-yl)nicotinamide (Example 910)

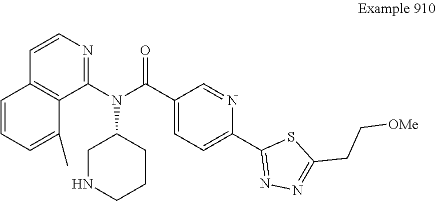 [SMILES: COCCc1nnc(-c2ccc(C(=O)N(c3nccc4cccc(C)c34)[C@@H]3CCCNC3)cn2)s1]

Example 910 was prepared according to the procedure described for Example 895 by substituting tert-butyl (R)-3-((6-chloro-8-methylisoquinolin-1-yl)amino)piperidine-1-carboxylate with tert-butyl (R)-3-((8-methylisoquinolin-1-yl)amino)piperidine-1-carboxylate and 2-methoxyacetohydrazide with 3-methoxypropanehydrazide, provided the title compound as a white solid. LC-MS: [M+H⁺]=489.2. ¹H NMR (400 MHz, CD₃OD): δ ppm 8.52-8.48 (m, 1H), 8.21 (s, 1H), 7.85-7.80 (m, 2H), 7.75-7.67 (m, 2H), 7.59-7.48 (m, 2H), 7.46-7.41 (m, 1H), 5.15-5.05 (m, 1H), 4.03-3.94 (m, 1H), 3.85-3.76 (m, 1H), 3.72-3.68 (m, 2H), 3.44-3.37 (m, 1H), 3.45 (s, 3H), 3.44-3.43 (m, 2H), 3.04-2.94 (m, 1H), 2.84-2.75 (m, 3H), 1.98-1.85 (m, 3H), 1.58-1.45 (m, 1H).

### Synthesis of N-(5-fluoro-6-methoxy-8-methyl-1-isoquinolyl)-6-(5-methyl-1,3,4-thiadiazol-2-yl)-N-[(3R)-3-piperidyl]pyridine-3-carboxamide (Example 911)

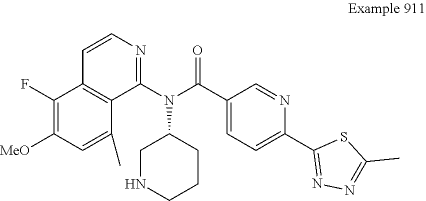 [SMILES: COc1cc(C)c2c(N(C(=O)c3ccc(-c4nnc(C)s4)nc3)[C@@H]3CCCNC3)nccc2c1F]

Example 911 was prepared according to the procedure described for Example 895 by substituting tert-butyl (R)-3-((6-chloro-8-methylisoquinolin-1-yl)amino)piperidine-1-carboxylate with tert-butyl (R)-3-((5-fluoro-6-methoxy-8-methylisoquinolin-1-yl)amino)piperidine-1-carboxylate and 2-methoxyacetohydrazide with acetohydrazide, provided the title compound as off white solid. LC-MS: [M+H]=493.2. ¹H NMR (400 MHz, CD₃OD): δ ppm 8.53-8.43 (m, 1H), 8.25 (s, 1H), 7.95-7.87 (m, 2H), 7.61 (br dd, J=1.0, 8.3 Hz, 1H), 7.43-7.35 (m, 1H), 5.12-5.03 (m, 1H), 3.96 (s, 3H), 3.88-3.82 (m, 1H), 3.82-3.74 (m, 1H), 3.39 (br d, J=13.3 Hz, 1H), 3.04-2.93 (m, 1H), 2.83-2.71 (m, 6H), 2.02-1.79 (m, 3H), 1.46 (dq, J=5.9, 12.0 Hz, 1H).

### Synthesis of 6-[5-(2-cyanoethyl)-1,3,4-thiadiazol-2-yl]-N-(3-methylthieno[3,2-c]pyridin-4-yl)-N-[(3R)-3-piperidyl]pyridine-3-carboxamide (Example 912)

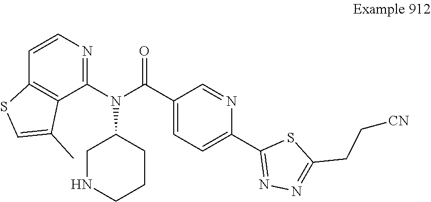 [SMILES: Cc1csc2ccnc(N(C(=O)c3ccc(-c4nnc(CCC#N)s4)nc3)[C@@H]3CCCNC3)c12]

Example 912 was prepared according to the procedure described for Example 895 by substituting tert-butyl (R)-3-((6-chloro-8-methylisoquinolin-1-yl)amino)piperidine-1-carboxylate with tert-butyl (R)-3-((3-methylthieno[3,2-c]pyridin-4-yl)amino)piperidine-1-carboxylate and 2-methoxyacetohydrazide with 3-cyanopropanehydrazide, provided the title compound as off white solid. LC-MS: [M+H⁺]=490.2. ¹H NMR (400 MHZ, CD₃OD): δ ppm 8.49-8.41 (m, 1H), 8.37 (d, J=1.8 Hz, 1H), 8.03-7.94 (m, 2H), 7.71 (dd, J=2.2, 8.3 Hz, 1H), 7.39 (s, 1H), 5.15 (tt, J=3.8, 11.5 Hz, 1H), 3.94 (br dd, J=3.4, 12.1 Hz, 1H), 3.81-3.70 (m, 1H), 3.52-3.45 (m, 2H), 3.38 (br s, 1H), 3.01 (t, J=7.1 Hz, 2H), 2.96-2.88 (m, 1H), 2.59-2.48 (m, 3H), 1.97-1.79 (m, 3H), 1.31 (br dd, J=4.3, 11.6 Hz, 1H).

### Synthesis of 6-[5-(2-methoxyethyl)-1,3,4-thiadiazol-2-yl]-N-(3-methylthieno[3,2-c]pyridin-4-yl)-N-[(3R)-3-piperidyl]pyridine-3-carboxamide (Example 913)

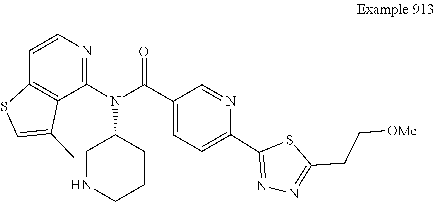 [SMILES: COCCc1nnc(-c2ccc(C(=O)N(c3nccc4scc(C)c34)[C@@H]3CCCNC3)cn2)s1]

Example 913 was prepared according to the procedure described for Example 895 by substituting tert-butyl (R)-3-((6-chloro-8-methylisoquinolin-1-yl)amino)piperidine-1-carboxylate with tert-butyl (R)-3-((3-methylthieno[3,2-c]pyridin-4-yl)amino)piperidine-1-carboxylate and 2-methoxyacetohydrazide with 3-methoxypropanehydrazide, provided the title compound as an off white solid. LC-MS: [M+H]=495.3. ¹H NMR (400 MHZ, CD₃OD): δ ppm 1.24-1.39 (m, 1H) 1.79-1.96 (m, 3H) 2.48-2.60 (m, 3H) 2.86-3.02 (m, 1H) 3.33-3.35 (m, 2H) 3.36-3.37 (m, 3H) 3.39 (br s, 1H) 3.72 (t, J=5.82 Hz, 2H) 3.74-3.78 (m, 1H) 3.88-3.99 (m, 1H) 5.09-5.20 (m, 1H) 7.37-7.44 (m, 1H) 7.70 (dd, J=8.25, 2.13 Hz, 1H) 7.95 (d, J=8.25 Hz, 1H) 8.02 (d, J=5.50 Hz, 1H) 8.36 (d, J=1.50 Hz, 1H) 8.43-8.50 (m, 1H).

### Synthesis of N-(6-methoxy-8-methyl-1-isoquinolyl)-4-(3-methyl-1, 2, 4-oxadiazol-5-yl)-N-[(3R)-3-piperidyl] benzamide (Example 914)

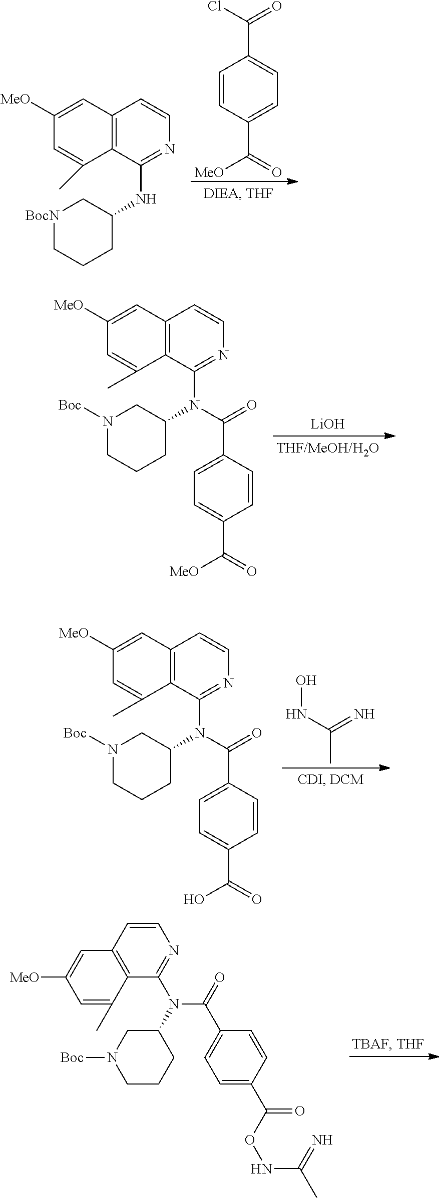

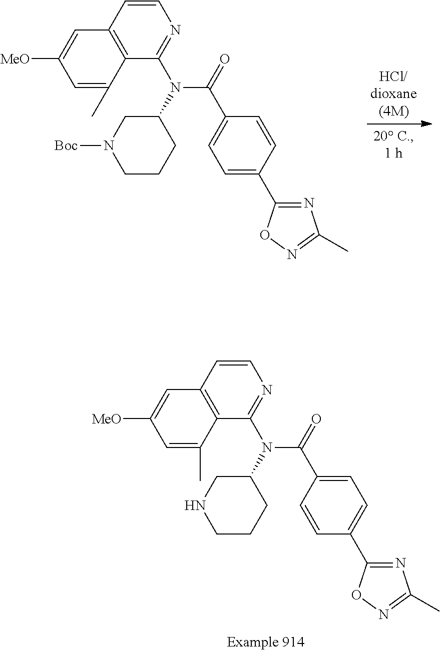 [SMILES: COc1cc(C)c2c(N(C(=O)c3ccc(-c4nc(C)no4)cc3)[C@@H]3CCCN(C(=O)OC(C)(C)C)C3)nccc2c1 ; COc1cc(C)c2c(N(C(=O)c3ccc(-c4nc(C)no4)cc3)[C@@H]3CCCNC3)nccc2c1]

### Step 1:

A mixture of tert-butyl (3R)-3-[(6-methoxy-8-methyl-1-isoquinolyl)amino] piperidine-1-carboxylate (500 mg, 1.35 mmol), methyl 4-chlorocarbonylbenzoate (801.96 mg, 4.04 mmol), DIEA (869.78 mg, 6.73 mmol) in THF (13 mL) was degassed with N₂, and then the mixture was stirred at 60° C. for 12 h under N₂ atmosphere. The reaction mixture was filtered and the filtrate was concentrated. The residue was purified by flash column using 20% EtOAc in petroleum ether to give tert-butyl (3R)-3-[(4-methoxycarbonylbenzoyl)-(6-methoxy-8-methyl-1-isoquinolyl) amino] piperidine-1-carboxylate (400 mg, 52%) as a yellowish oil.

### Step 2:

To a solution of tert-butyl (3R)-3-[(4-methoxycarbonylbenzoyl)-(6-methoxy-8-methyl-1-isoquinolyl) amino] piperidine-1-carboxylate (450 mg, 843.31 μmol) in H₂O (3 mL), THF (3 mL) and MeOH (3 mL) was added LiOH·H₂O (88.46 mg, 2.11 mmol). The mixture was stirred at 20° C. for 12 h. The reaction mixture was concentrated. The residue was diluted with EtOAc (10 mL) and water (10 mL). The mixture was extracted with EtOAc (20 mL×3). The aqueous layer was acidified to pH=4 with 1N HCl. The mixture was extracted with EtOAc (20 mL×3). The combined organic layers were washed with brine (10 mL), dried over Na₂SO₄, filtered and concentrated under reduced pressure to give crude t 4-[[(3R)-1-tert-butoxycarbonyl-3-piperidyl]-(6-methoxy-8-methyl-1-isoquinolyl) carbamoyl] benzoic acid (300 mg, 65%) as a yellowish oil.

### Step 3:

To a solution of 4-[[(3R)-1-tert-butoxycarbonyl-3-piperidyl]-(6-methoxy-8-methyl-1-isoquinolyl) carbamoyl] benzoic acid (300 mg, 548.51 μmol) in DCM (4 mL) was added CDI (93.39 mg, 575.94 μmol). After stirring for 0.5 h, N-hydroxyacetamidine (42.67 mg, 575.94 μmol) was added to the above mixture. The mixture was stirred at 20° C. for 11.5 h. The reaction mixture was filtered and the filtrate was concentrated. The residue was purified by prep-TLC using EtOAc to afford tert-butyl (3R)-3-[[4-(ethanimidoylamino) oxycarbonylbenzoyl]-(6-methoxy-8-methyl-1-isoquinolyl) amino] piperidine-1-carboxylate (170 mg, 50%) was obtained as a colorless oil.

### Step 4:

To a solution of tert-butyl (3R)-3-[[4-(ethanimidoylamino) oxycarbonylbenzoyl]-(6-methoxy-8-methyl-1-isoquinolyl) amino] piperidine-1-carboxylate (170 mg, 274.64 μmol) in THF (2 mL) was added TBAF (1M, 1.10 mL). The mixture was stirred at 20° C. for 12 h. The reaction mixture was concentrated under reduced pressure. The residue was diluted with water (10 mL) and extracted with EtOAc (3×3 mL). The combined organic layer was washed with brine (3 mL), dried over Na₂SO₄, filtered and concentrated to give crude tert-butyl (3R)-3-[(6-methoxy-8-methyl-1-isoquinolyl)-[4-(3-methyl-1, 2, 4-oxadiazol-5-yl) benzoyl] amino] piperidine-1-carboxylate (110 mg, 68%) as a yellowish oil.

### Step 5:

Example 914 was prepared according to the Boc removal procedure described for Example 97 to provide the title compound hydrochloride salt as a white solid. LC-MS: [M+H⁺]=558.3. ¹H NMR (400 MHZ, CD₃OD): δ ppm 8.40 (d, J=5.6 Hz, 1H), 7.77-7.68 (m, 3H), 7.27 (d, J=8.4 Hz, 2H), 7.12-7.01 (m, 2H), 5.11-4.98 (m, 1H), 4.02-3.92 (m, 1H), 3.90-3.76 (m, 4H), 3.39 (br d, J=12.8 Hz, 1H), 3.08-2.93 (m, 1H), 2.79-2.68 (m, 3H), 2.35 (s, 3H), 2.22-1.81 (m, 3H), 1.59-1.43 (m, 1H).

### Synthesis of N-(6, 8-dimethyl-1-isoquinolyl)-4-(3-methyl-1, 2, 4-oxadiazol-5-yl)-N-[(3R)-3-piperidyl] benzamide (Example 915)

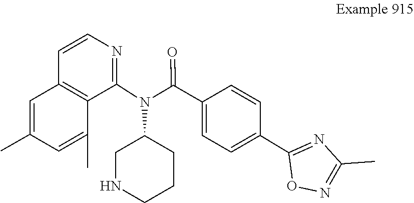

Example 915 was prepared according to the procedure described for Example 914 by substituting tert-butyl (R)-3-((6-methoxy-8-methylisoquinolin-1-yl)amino)piperidine-1-carboxylate with tert-butyl (R)-3-((6,8-dimethylisoquinolin-1-yl)amino)piperidine-1-carboxylate, provided the title compound as a white solid. LC-MS: [M+H⁺]=442.3. ¹H NMR (400 MHZ, METHANOL-d₄): δ ppm 8.44 (br d, J=5.4 Hz, 1H), 7.71 (br d, J=7.4 Hz, 3H), 7.46 (s, 1H), 7.32-7.19 (m, 3H), 5.12-5.01 (m, 1H), 3.96 (br d, J=11.1 Hz, 1H), 3.87-3.76 (m, 1H), 3.39 (br d, J=12.6 Hz, 1H), 3.08-2.92 (m, 1H), 2.83-2.69 (m, 3H), 2.35 (s, 6H), 2.00-1.79 (m, 3H), 1.49 (br dd, J=5.7, 10.8 Hz, 1H).

### Synthesis of N-(6,8-dimethyl-1-isoquinolyl)-6-(3-methyl-1,2,4-oxadiazol-5-yl)-N-[(3R)-3-piperidyl]pyridine-3-carboxamide (Example 916)

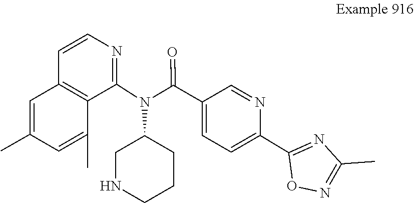

Example 916 was prepared according to the procedure described for Example 914 by substituting tert-butyl (R)-3-((6-methoxy-8-methylisoquinolin-1-yl)amino)piperidine-1-carboxylate with tert-butyl (R)-3-((6,8-dimethylisoquinolin-1-yl)amino)piperidine-1-carboxylate and methyl 4-(chlorocarbonyl) benzoate with methyl 5-(chlorocarbonyl)picolinate, provided the title compound as a white solid. LC-MS: [M+H⁺]=443.2. ¹H NMR (400 MHZ, DMSO-d6): δ ppm 8.94-8.86 (m, 1H), 8.49 (d, J=5.8 Hz, 1H), 8.36 (s, 1H), 7.94-7.76 (m, 2H), 7.70 (dd, J=1.5, 8.4 Hz, 1H), 7.56 (s, 1H), 7.32 (s, 1H), 5.02-4.87 (m, 1H), 3.85-3.78 (m, 1H), 3.26 (br s, 2H), 2.82 (br dd, J=5.5, 10.1 Hz, 1H), 2.67 (s, 3H), 2.37-2.32 (m, 7H), 1.88-1.62 (m, 3H), 1.25-1.12 (m, 1H).

### Synthesis of N-(6-chloro-8-methyl-1-isoquinolyl)-6-(5-methyl-1,3,4-oxadiazol-2-yl)-N-[(3R)-3-piperidyl]pyridine-3-carboxamide (Example 917)

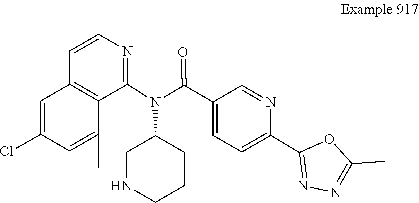 [SMILES: Cc1nnc(-c2ccc(C(=O)N(c3nccc4cc(Cl)cc(C)c34)[C@@H]3CCCNC3)cn2)o1]

Example 917 was prepared according to the procedure described for Example 111 by substituting tert-butyl (R)-3-((8-methylisoquinolin-1-yl)amino)piperidine-1-carboxylate with tert-butyl (R)-3-((6-chloro-8-methylisoquinolin-1-yl)amino)piperidine-1-carboxylate and 4-(1-methyl-1H-1,2,3-triazol-4-yl) benzoic acid with 6-(5-methyl-1,3,4-oxadiazol-2-yl)nicotinic acid, provided the title compound as an off white solid. LC-MS: [M+H]=463.3. ¹H NMR (400 MHZ, CD₃OD): δ ppm 1.25-1.40 (m, 1H) 1.71 (br s, 3H) 2.47-2.60 (m, 4H) 2.74-2.83 (m, 3H) 2.96-3.07 (m, 1H) 3.47-3.51 (m, 1H) 3.70 (br d, J=12.26 Hz, 1H) 4.71-4.81 (m, 1H) 7.44 (s, 1H) 7.65-7.70 (m, 1H) 7.73-7.77 (m, 2H) 7.81 (d, J=8.13 Hz, 1H) 8.31 (d, J=1.38 Hz, 1H) 8.55 (d, J=5.63 Hz, 1H).

### Synthesis of N-(6-fluoro-8-methyl-1-isoquinolyl)-6-(5-methyl-1,3,4-oxadiazol-2-yl)-N-[(3R)-3-piperidyl]pyridine-3-carboxamide (Example 918)

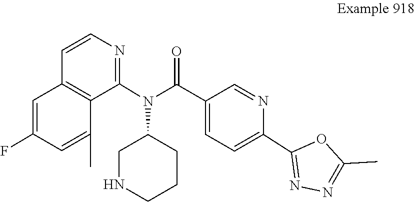 [SMILES: Cc1nnc(-c2ccc(C(=O)N(c3nccc4cc(F)cc(C)c34)[C@@H]3CCCNC3)cn2)o1]

Example 918 was prepared according to the procedure described for Example 111 by substituting tert-butyl (R)-3-((8-methylisoquinolin-1-yl)amino)piperidine-1-carboxylate with tert-butyl (R)-3-((6-fluoro-8-methylisoquinolin-1-yl)amino)piperidine-1-carboxylate and 4-(1-methyl-1H-1,2,3-triazol-4-yl) benzoic acid with 6-(5-methyl-1,3,4-oxadiazol-2-yl)nicotinic acid, provided the title compound as an off white solid. LC-MS: [M+H]=447.3. ¹H NMR (400 MHz, CD₃OD): δ ppm 1.27-1.40 (m, 1H) 1.61-1.81 (m, 3H) 2.47-2.59 (m, 4H) 2.76-2.82 (m, 3H) 2.96-3.04 (m, 1H) 3.35-3.40 (m, 1H) 3.69 (br d, J=11.63 Hz, 1H) 4.70-4.81 (m, 1H) 7.23-7.31 (m, 1H) 7.37 (dd, J=8.50, 2.50 Hz, 1H) 7.63-7.69 (m, 1H) 7.74-7.82 (m, 2H) 8.30 (d, J=2.00 Hz, 1H) 8.45-8.52 (m, 1H).

### Synthesis of 6-(5-methyl-1,3,4-oxadiazol-2-yl)-N-(3-methylthieno[3,2-c]pyridin-4-yl)-N-[(3R)-3-piperidyl]pyridine-3-carboxamide (Example 919)

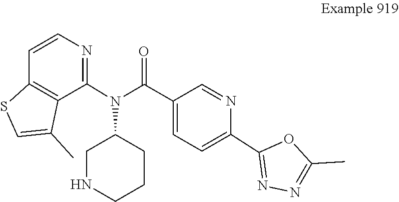 [SMILES: Cc1nnc(-c2ccc(C(=O)N(c3nccc4scc(C)c34)[C@@H]3CCCNC3)cn2)o1]

Example 919 was prepared according to the procedure described for Example 111 by substituting tert-butyl (R)-3-((8-methylisoquinolin-1-yl)amino)piperidine-1-carboxylate with tert-butyl (R)-3-((3-methylthieno[3,2-c]pyridin-4-yl)amino)piperidine-1-carboxylate and 4-(1-methyl-1H-1,2,3-triazol-4-yl)benzoic acid with 6-(5-methyl-1,3,4-oxadiazol-2-yl)nicotinic acid, provided the title compound as off white solid. LC-MS: [M+H]=435.3. ¹H NMR (400 MHZ, CD₃OD): δ ppm 1.04-1.19 (m, 1H) 1.54-1.78 (m, 3H) 2.39-2.48 (m, 1H) 2.48-2.56 (m, 4H) 2.57 (s, 3H) 2.97 (br d, J=12.51 Hz, 1H) 3.64 (br d, J=10.76 Hz, 1H) 4.72-4.83 (m, 1H) 7.33-7.40 (m, 1H) 7.74-7.81 (m, 1H) 7.84-7.89 (m, 1H) 7.91-7.95 (m, 1H) 8.39-8.48 (m, 2H).

### Synthesis of N-(6-fluoro-8-methyl-1-isoquinolyl)-6-[1-(2-methoxyethyl)triazol-4-yl]-N-[(3R)-3-piperidyl]pyridine-3-carboxamide (Example 920)

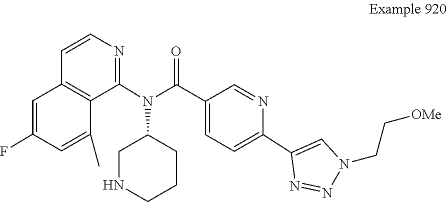 [SMILES: COCCn1cc(-c2ccc(C(=O)N(c3nccc4cc(F)cc(C)c34)[C@@H]3CCCNC3)cn2)nn1]

Example 920 was prepared according to the procedure described for Example 111 by substituting tert-butyl (R)-3-((8-methylisoquinolin-1-yl)amino)piperidine-1-carboxylate with tert-butyl (R)-3-((6-fluoro-8-methylisoquinolin-1-yl)amino)piperidine-1-carboxylate and 4-(1-methyl-1H-1,2,3-triazol-4-yl)benzoic acid with 6-(1-(2-methoxyethyl)-1H-1,2,3-triazol-4-yl)nicotinic acid, provided the title compound as an off white solid. LC-MS: [M+H]=490.3. ¹H NMR (400 MHZ, CD₃OD): δ ppm 8.54 (d, J=5.5 Hz, 1H), 8.43-8.38 (m, 1H), 8.17 (s, 1H), 7.86 (d, J=5.5 Hz, 1H), 7.77 (d, J=8.3 Hz, 1H), 7.65 (dd, J=1.4, 8.3 Hz, 1H), 7.43 (br dd, J=2.5, 8.3 Hz, 1H), 7.29 (br d, J=9.4 Hz, 1H), 5.15-5.04 (m, 1H), 4.59 (t, J=4.9 Hz, 2H), 3.98 (br d, J=12.0 Hz, 1H), 3.82 (br d, J=11.3 Hz, 1H), 3.77 (t, J=4.9 Hz, 2H), 3.54 (br d, J=12.6 Hz, 1H), 3.40 (br d, J=12.4 Hz, 3H), 3.07-2.94 (m, 1H), 2.84-2.70 (m, 3H), 2.00-1.81 (m, 3H), 1.56-1.44 (m, 1H).

### Synthesis of N-(6-chloro-8-methyl-1-isoquinolyl)-6-[1-(2-methoxyethyl)triazol-4-yl]-N-[(3R)-3-piperidyl]pyridine-3-carboxamide (Example 921)

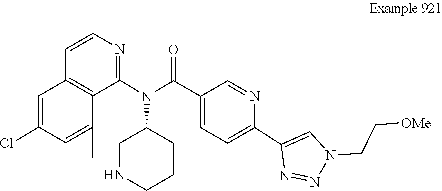 [SMILES: COCCn1cc(-c2ccc(C(=O)N(c3nccc4cc(Cl)cc(C)c34)[C@@H]3CCCNC3)cn2)nn1]

Example 921 was prepared according to the procedure described for Example 111 by substituting tert-butyl (R)-3-((8-methylisoquinolin-1-yl)amino)piperidine-1-carboxylate with tert-butyl (R)-3-((6-chloro-8-methylisoquinolin-1-yl)amino)piperidine-1-carboxylate and 4-(1-methyl-1H-1,2,3-triazol-4-yl) benzoic acid with 6-(1-(2-methoxyethyl)-1H-1,2,3-triazol-4-yl)nicotinic acid, provided the title compound as an off white solid. LC-MS: [M+H]=506.2. ¹H NMR (400 MHz, CD₃OD): δ ppm 8.57 (d, J=5.5 Hz, 1H), 8.34 (s, 1H), 8.14 (s, 1H), 7.82 (d, J=5.6 Hz, 1H), 7.80-7.76 (m, 1H), 7.72 (d, J=8.1 Hz, 1H), 7.57 (br d, J=7.8 Hz, 1H), 7.47-7.39 (m, 1H), 5.14-5.05 (m, 1H), 4.58 (t, J=5.0 Hz, 2H), 3.97 (br dd, J=2.3, 10.9 Hz, 1H), 3.80 (s, 1H), 3.77 (t, J=4.9 Hz, 2H), 3.60-3.51 (m, 1H), 3.44-3.37 (m, 3H), 3.04-2.91 (m, 1H), 2.83-2.70 (m, 3H), 2.00-1.83 (m, 3H), 1.51 (br dd, J=4.7, 12.1 Hz, 1H).

### Synthesis of (R)-4-(3,4-dihydro-2H-pyrano[3,2-c]pyridin-8-yl)-N-(8-methylisoquinolin-1-yl)-N-(piperidin-3-yl)benzamide (Example 922)

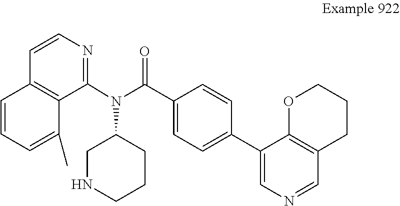 [SMILES: Cc1cccc2ccnc(N(C(=O)c3ccc(-c4cncc5c4OCCC5)cc3)[C@@H]3CCCNC3)c12]

Example 922 was prepared according to the procedure described for Example 111 by substituting 4-bromo-1-methyl-1H-1,2,3-triazole with 8-iodo-3,4-dihydro-2H-pyrano[3,2-c]pyridine, provided the title compound as white solid. LC-MS: [M+H]=479.2. ¹H NMR (400 MHZ, METHANOL-d4): δ ppm 1.85 (br s, 1H) 1.88-1.98 (m, 2H) 2.01-2.19 (m, 3H) 2.73-2.84 (m, 3H) 2.93 (br t, J=6.40 Hz, 2H) 3.36-3.43 (m, 2H) 3.79 (t, J=11.42 Hz, 1H) 3.92-4.01 (m, 1H) 4.44 (t, J=5.27 Hz, 2H) 5.04-5.14 (m, 1H) 7.15-7.27 (m, 4H) 7.36-7.45 (m, 1H) 7.46-7.56 (m, 1H) 7.66-7.75 (m, 1H) 7.76-7.85 (m, 1H) 8.24 (s, 1H) 8.39 (s, 1H) 8.44-8.53 (m, 1H).

### Synthesis of 2-fluoro-4-(5-methyl-6,7-dihydro-4H-triazolo[1,5-a]pyrazin-3-yl)-N-(3-methylthieno[3,2-c]pyridin-4-yl)-N-[(3R)-3-piperidyl]benzamide (Example 923)

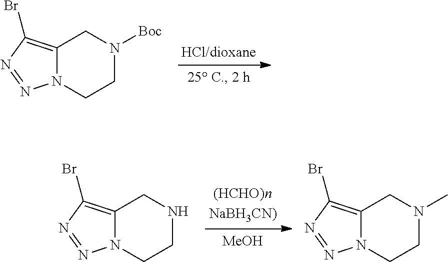 [SMILES: "MeOH" ; CC(C)(C)OC(=O)N1CCn2nnc(Br)c2C1 ; Brc1nnn2c1CNCC2 ; CN1CCn2nnc(Br)c2C1]

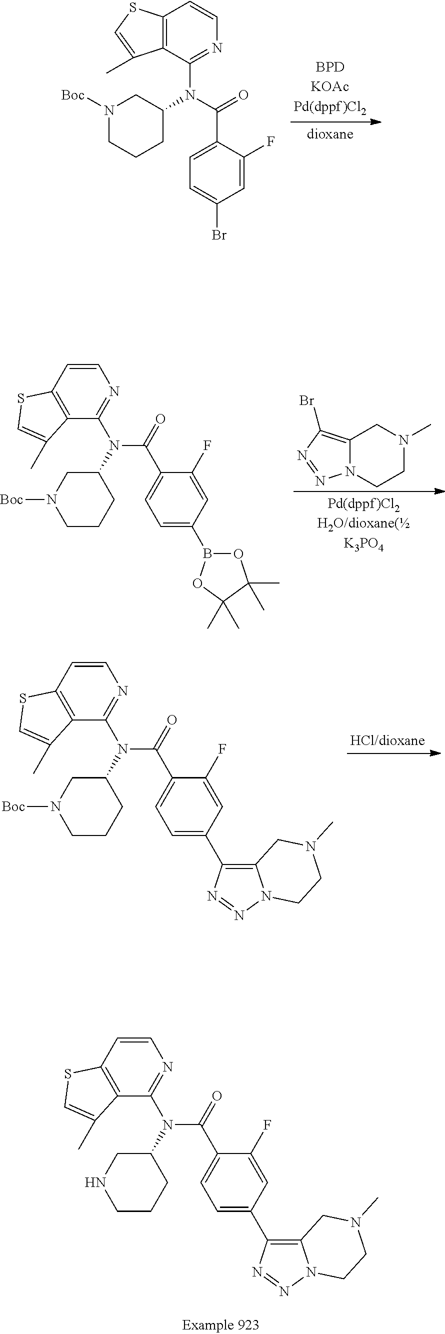 [SMILES: Cc1csc2ccnc(N(C(=O)c3ccc(Br)cc3F)[C@@H]3CCCN(C(=O)OC(C)(C)C)C3)c12 ; Cc1csc2ccnc(N(C(=O)c3ccc(B4OC(C)(C)C(C)(C)O4)cc3F)[C@@H]3CCCN(C(=O)OC(C)(C)C)C3)c12 ; CN1CCn2nnc(Br)c2C1 ; Cc1csc2ccnc(N(C(=O)c3ccc(-c4nnn5c4CN(C)CC5)cc3F)[C@@H]3CCCN(C(=O)OC(C)(C)C)C3)c12 ; Cc1csc2ccnc(N(C(=O)c3ccc(-c4nnn5c4CN(C)CC5)cc3F)[C@@H]3CCCNC3)c12]

### Step 1:

A mixture of tert-butyl 3-bromo-6,7-dihydro-4H-triazolo[1,5-a]pyrazine-5-carboxylate (400 mg, 1.32 mmol), HCl/dioxane (4 M, 4 mL) in dioxane (4 mL) was degassed and purged with N₂ for 3 times, and then the mixture was stirred at 20° C. for 1 h under N₂ atmosphere. The reaction mixture was concentrated under reduced pressure to provide crude 3-bromo-4,5,6,7-tetrahydrotriazolo[1,5-a]pyrazine (240 mg, 89%) was used for the next step directly.

### Step 2:

To a solution of 3-bromo-4,5,6,7-tetrahydrotriazolo[1,5-a]pyrazine (240 mg, 1.18 mmol) in MeOH (5 mL) was added (HCHO)n (531.92 mg, 5.91 mmol) and NaBH₃CN (148.56 mg, 2.36 mmol). The mixture was stirred at 20° C. for 2 h. The reaction mixture was filtered and concentrated. The residue was purified by flash column using EtOAc to give 3-bromo-5-methyl-6,7-dihydro-4H-triazolo[1,5-a]pyrazine (200 mg, 77%) as a white solid.

### Step 3:

A mixture of tert-butyl (3R)-3-[(4-bromo-2-fluoro-benzoyl)-(3-methylthieno[3,2-c]pyridin-4-yl)amino]piperidine-1-carboxylate (300 mg, 546.98 μmol), BPD (416.70 mg, 1.64 mmol), Pd(dppf)Cl₂ (40.02 mg, 54.70 μmol) and KOAc (161.04 mg, 1.64 mmol) in dioxane (3 mL) was degassed with N₂, and then the mixture was stirred at 100° C. for 12 h under N₂ atmosphere. The reaction mixture was filtered and concentrated. The residue was purified by flash column using 10% EtOAc in petroleum ether to give tert-butyl (3R)-3-[[2-fluoro-4-(4,4,5,5-tetramethyl-1,3,2-dioxaborolan-2-yl)benzoyl]-(3-methylthieno[3,2-c]pyridin-4-yl)amino]piperidine-1-carboxylate (350 mg, 82%) as a white solid.

### Step 4:

A mixture of tert-butyl (3R)-3-[[2-fluoro-4-(4,4,5,5-tetramethyl-1,3,2-dioxaborolan-2-yl)benzoyl]-(3-methylthieno[3,2-c]pyridin-4-yl)amino]piperidine-1-carboxylate (200 mg, 335.83 μmol), Pd(dppf)Cl₂ (24.57 mg, 33.58 μmol), K₃PO₄ (213.86 mg, 1.01 mmol) and 3-bromo-5-methyl-6,7-dihydro-4H-triazolo[1,5-a]pyrazine (72.90 mg, 335.83 μmol) in dioxane (4 mL)/H₂O (0.5 mL) was degassed and purged with N₂ for 3 times, and then the mixture was stirred at 100° C. for 12 h under N₂ atmosphere. The reaction mixture was filtered and concentrated. The residue was purified by flash column using 10% EtOAc in petroleum ether to give tert-butyl (3R)-3-[[2-fluoro-4-(5-methyl-6,7-dihydro-4H-triazolo[1,5-a]pyrazin-3-yl)benzoyl]-(3-methylthieno[3,2-c]pyridin-4-yl)amino]piperidine-1-carboxylate (90 mg, 28%) as a white oil.

### Step 5:

A mixture of tert-butyl (3R)-3-[[2-fluoro-4-(5-methyl-6,7-dihydro-4H-triazolo[1,5-a]pyrazin-3-yl)benzoyl]-(3-methylthieno[3,2-c]pyridin-4-yl)amino]piperidine-1-carboxylate (90 mg, 77.26 μmol) and 4 M HCl/dioxane (1 mL) was stirred at 20° C. for 1 h. The reaction mixture was filtered and concentrated under reduced pressure to give a residue. The residue was purified by reverse phase prep-HPLC to give 2-fluoro-4-(5-methyl-6,7-dihydro-4H-triazolo[1,5-a]pyrazin-3-yl)-N-(3-methylthieno[3,2-c]pyridin-4-yl)-N-[(3R)-3-piperidyl]benzamide (16.7 mg, 42%) as a white solid. LC-MS: [M+H⁺]=506.3. ¹H NMR (400 MHZ, DMSO-d₆): δ ppm 9.23-9.10 (m, 1H), 9.06-8.91 (m, 1H), 8.38 (d, J=5.4 Hz, 1H), 8.04 (d, J=5.4 Hz, 1H), 7.60 (s, 1H), 7.30 (br d, J=11.3 Hz, 1H), 7.20 (br d, J=8.1 Hz, 1H), 7.11-7.01 (m, 1H), 4.98-4.90 (m, 1H), 4.81-4.60 (m, 3H), 3.51-3.42 (m, 1H), 3.22 (br d, J=11.4 Hz, 1H), 2.93 (br s, 3H), 2.80-2.65 (m, 3H), 2.60 (br d, J=14.4 Hz, 2H), 2.53 (s, 3H), 1.85-1.67 (m, 3H), 1.11-0.99 (m, 1H).

### Synthesis of (R)—N-(3-methylpyridin-2-yl)-N-(piperidin-3-yl)-4-(pyrimidin-2-ylamino)benzamide (Example 924)

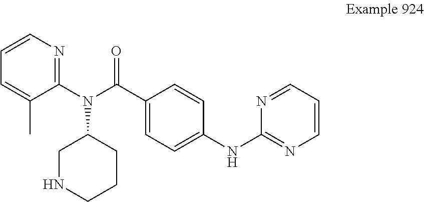 [SMILES: Cc1cccnc1N(C(=O)c1ccc(Nc2ncccn2)cc1)[C@@H]1CCCNC1]

Example 924 was prepared following the procedure for example 309 by substituting 1-chloro-8-methylisoquinoline with 2-chloro-3-methylpyridine to afford the title compound as an off white solid. LC-MS: [M+H⁺]=389.0.

### Synthesis of (R)—N-(3-chloropyridin-2-yl)-N-(piperidin-3-yl)-4-(pyrimidin-2-ylamino)benzamide (Example 925)

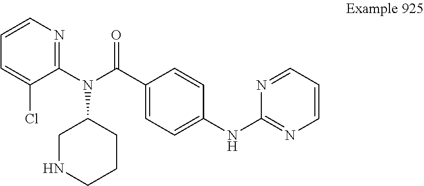 [SMILES: O=C(c1ccc(Nc2ncccn2)cc1)N(c1ncccc1Cl)[C@@H]1CCCNC1]

Example 925 was prepared following the procedure for example 309 by substituting 1-chloro-8-methylisoquinoline with 2,3-dichloro-pyridine to afford the title compound as an off white solid. LC-MS: [M+H⁺]=409.0.

### (R)-2-fluoro-N-(1-methyl-1H-pyrrolo[2,3-c]pyridin-7-yl)-N-(piperidin-3-yl)-4-(pyrimidin-2-ylamino)benzamide (Example 926)

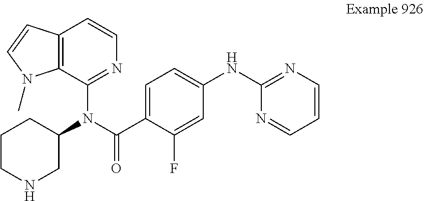 [SMILES: Cn1ccc2ccnc(N(C(=O)c3ccc(Nc4ncccn4)cc3F)[C@@H]3CCCNC3)c21]

Example 926 was synthesized following the procedure for example 309 by substituting 1-chloro-8-methylisoquinoline with 7-chloro-1-methyl-1H-pyrrolo[2,3-c]pyridine to afford the title compound as an off-white solid. LC-MS: [M+H⁺]=446.65. ¹H NMR (DMSO-d₆, 400 MHz) δ ppm 9.90 (bs, 1H), 8.49-8.48 (m, 2H), 8.08-8.01 (m, 1H), 7.70-7.50 (m, 4H), 7.06-7.84 (m, 2H), 6.47-6.44 (m, 1H), 4.97-4.95 (m, 1H), 3.90 (s, 3H), 3.72-3.69 (m, 1H), 3.22-3.18 (m, 1H), 2.79-2.71 (m, 2H), 1.36-1.23 (m, 2H), 1.05-1.01 (m, 1H), 0.89-0.84 (m, 1H).

### Synthesis (R)-2-fluoro-N-(isoquinolin-1-yl)-N-(piperidin-3-yl)-4-(pyrimidin-2-ylamino)benzamide (Example 927)

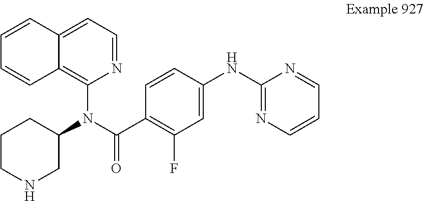 [SMILES: O=C(c1ccc(Nc2ncccn2)cc1F)N(c1nccc2ccccc12)[C@@H]1CCCNC1]

Example 927 was synthesized following the procedure for example 210 by substituting 1-chloro-8-methylisoquinoline with 1-chloro-isoquinoline to afford the title compound as an off white solid. LC-MS: [M+H⁺]=454.0 ¹H NMR (CD₃OD, 400 MHZ) δ ppm 8.58 (d, J=5.2 Hz, 2H), 8.46 (d, J=5.6 Hz, 1H), 8.14 (b, 1H), 7.9-7.65 (m, 4H), 7.5-7.4 (m, 1H), 7.15-6.9 (m, 3H), 5.35-5.2 (b, 1H), 3.85-3.6 (m, 2H), 3.4-3.3 (m, 1H), 2.9-2.8 (b, 1H), 2-1.8 (m, 3H), 1.35-1.2 (b, 1H).

### Synthesis of (R)-2-fluoro-4-(methyl(pyrimidin-2-yl)amino)-N-(8-methylisoquinolin-1-yl)-N-(piperidin-3-yl)benzamide (Example 928)

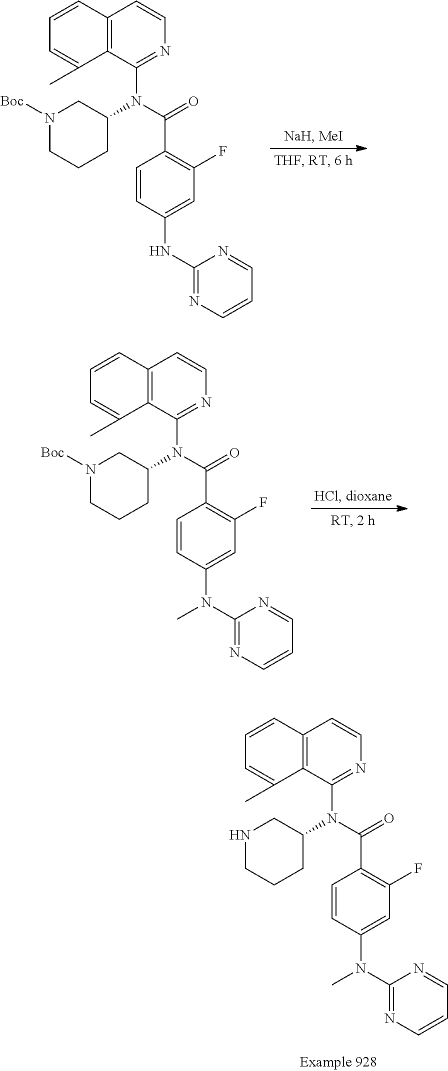

### Step 1:

To a solution of tert-butyl (R)-3-(2-fluoro-N-(8-methylisoquinolin-1-yl)-4-(pyrimidin-2-ylamino)benzamido)piperidine-1-carboxylate (25 mg, 44.9 μmol) in THF (1.5 mL) at 0° C. was added NaH (5.26 mg, 131.43 μmol) under N₂ atmosphere. The mixture was stirred at 20° C. for 0.5 h. Iodomethane (24.87 mg, 175.24 μmol) was then added to the mixture at 20° C. under N₂ atmosphere, and the mixture was stirred at 20° C. for 5.5 h. The mixture was poured into water (1 mL), and extracted with EtOAc (1×2 mL). The combined organic layer was washed with brine, dried over Na₂SO₄, and evaporated. The residue was purified by prep-TLC to give tert-butyl (R)-3-(2-fluoro-4-(methyl(pyrimidin-2-yl)amino)-N-(8-methylisoquinolin-1-yl)benzamido)-piperidine-1-carboxylate (20 mg) as a solid.

### Step 2:

Example was prepared according to the Boc removal procedure described for Example 97 to give the title compound hydrochloride salt as a white solid. LC-MS: [M+H⁺]=471.2. ¹H NMR (400 MHZ, CD₃OD) δ ppm 8.55-8.52 (m, 2H), 8.45-8.43 (m, 1H), 7.79-7.42 (m, 4H), 7.09-7.01 (m, 3H), 6.91-6.70 (m, 1H), 5.12-5.06 (m, 1H), 4.03-3.95 (m, 1H), 3.86-3.74 (m, 2H), 3.43-3.40 (m, 1H), 3.25-2.95 (m, 2H), 2.85 (s, 3H), 2.75-2.66 (m, 1H), 2.05-1.93 (m, 3H), 1.64-1.45 (m, 1H).

### Synthesis of methyl (R)-2-((3-fluoro-4-((8-methylisoquinolin-1-yl)(piperidin-3-yl)carbamoyl)phenyl)amino)pyrimidine-5-carboxylate (Example 929)

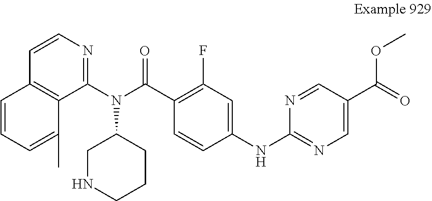 [SMILES: COC(=O)c1cnc(Nc2ccc(C(=O)N(c3nccc4cccc(C)c34)[C@@H]3CCCNC3)c(F)c2)nc1]

Example 929 was prepared according to the procedure described for Example 131 by substituting 3-amino-1-methyl-pyridin-2-one with methyl 2-aminopyrimidine-5-carboxylate to afford the title compound as an off-white solid. LC-MS: [M+H⁺]=515.3. ¹H NMR (CD₃OD, 400 MHZ): δ 8.89 (s, 2H), 8.43-8.40 (m, 1H), 7.74-7.70 (m, 1H), 7.67-7.62 (m, 1H), 7.55-7.43 (m, 3H), 6.91 (d, J=1.6 Hz, J=8.4 Hz, 1H), 6.70-6.66 (m, 1H), 5.10-5.02 (m, 1H), 3.97-3.92 (m, 1H), 3.87 (s, 3H), 3.84-3.78 (m, 1H), 3.43-3.38 (m, 1H), 3.02-2.98 (m, 1H), 2.80 (s, 3H), 1.94-1.88 (m, 3H), 1.62-1.55 (m, 1H).

### Synthesis of (R)-2-fluoro-N-(piperidin-3-yl)-N-(3-(prop-1-en-2-yl)pyridin-2-yl)-4-(pyrimidin-2-ylamino)benzamide (Example 930)

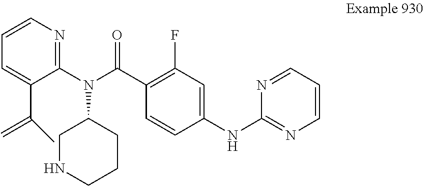 [SMILES: C=C(C)c1cccnc1N(C(=O)c1ccc(Nc2ncccn2)cc1F)[C@@H]1CCCNC1]

Example 930 was prepared according to the procedure described for Example 210 by substituting (R)-8-methyl-N-(piperidin-3-yl)isoquinolin-1-amine with (R)—N-(piperidin-3-yl)-3-(prop-1-en-2-yl)pyridin-2-amine to afford the title compound as an off-white solid. LC-MS: [M+H⁺]=433.2. ¹H NMR (CD₃OD, 400 MHZ): δ ppm 8.70-8.67 (m, 2H), 8.55-8.51 (m, 1H), 7.64-7.53 (m, 2H), 7.38-7.37 (br s, 1H), 7.22-7.13 (m, 2H), 7.09-6.97 (m, 1H), 5.28-5.27 (m, 1H), 4.96-4.94 (m, 1H), 4.86-4.85 (m, 1H), 3.82-3.58 (m, 2H), 3.40-3.31 (m, 1H), 3.01-2.95 (m, 1H), 2.05-1.71 (m, 7H).

### Synthesis of (R)-2-fluoro-N-(3-methylthieno[3,2-c]pyridin-4-yl)-N-(piperidin-3-yl)-4-(pyrimidin-2-ylamino)benzamide (Example 931)

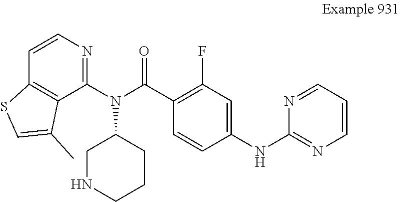 [SMILES: Cc1csc2ccnc(N(C(=O)c3ccc(Nc4ncccn4)cc3F)[C@@H]3CCCNC3)c12]

Example 931 was prepared according to the procedure described for Example 210 by substituting (R)-8-methyl-N-(piperidin-3-yl)isoquinolin-1-amine with (R)-3-methyl-N-(piperidin-3-yl)thieno[3,2-c]pyridin-4-amine to afford the title compound as an off-white solid. LC-MS: [M+H⁺]=463.2. ¹H NMR (CD₃OD, 400 MHZ): δ ppm 8.48 (d, J=4.9 Hz, 2H), 8.38 (d, J=5.5 Hz, 1H), 7.93 (d, J=5.5 Hz, 1H), 7.59 (dd, J=1.4, 13.4 Hz, 1H), 7.40 (s, 1H), 6.99-6.81 (m, 3H), 5.12-5.04 (m, 1H), 3.97-3.88 (m, 1H), 3.76-3.67 (m, 1H), 3.40-3.34 (m, 2H), 2.99-2.86 (m, 1H), 2.70-2.49 (m, 3H), 2.03-1.75 (m, 2H), 1.37-1.21 (m, 1H).

### Synthesis of (R)-4-((4-(1H-indazol-5-yl)pyrimidin-2-yl)amino)-2-fluoro-N-(8-methylisoquinolin-1-yl)-N-(piperidin-3-yl)benzamide (Example 932)

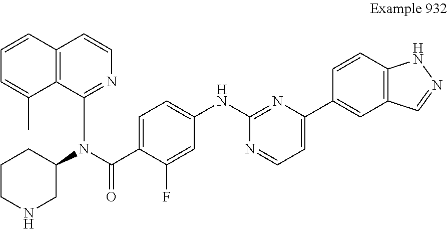 [SMILES: Cc1cccc2ccnc(N(C(=O)c3ccc(Nc4nccc(-c5ccc6[nH]ncc6c5)n4)cc3F)[C@@H]3CCCNC3)c12]

Example 932 was synthesized following the procedure for Example 131 by substituting 3-amino-1-methylpyridin-2 (1H)-one with tert-butyl 5-(2-aminopyrimidin-4-yl)-1H-indazole-1-carboxylate to afford the title compound as an off-white solid. LC-MS: [M+H⁺]=573.3. ¹H NMR (CD₃OD, 400 MHZ): δ ppm 8.77 (bs, 1H), 8.46-8.43 (m, 1H), 8.36-8.33 (m, 2H), 8.29 (d, J=6.8 Hz, 1H), 7.76-7.65 (m, 3H), 7.56-7.49 (m, 3H), 7.33-7.30 (m, 1H), 7.21-7.19 (m, 1H), 7.07-7.03 (m, 1H), 5.09-5.02 (m, 1H), 4.01-3.97 (m, 1H), 3.87-3.82 (m, 1H), 3.48-3.47 (m, 2H), 3.13-3.12 (m, 1H), 2.87 (s, 3H), 2.90-2.87 (m, 1H), 1.98-1.93 (m, 2H).

### Synthesis of (R)-4-((4-(4-acetylpiperazin-1-yl)pyrimidin-2-yl)amino)-2-fluoro-N-(8-methylisoquinolin-1-yl)-N-(piperidin-3-yl)benzamide (Example 933)

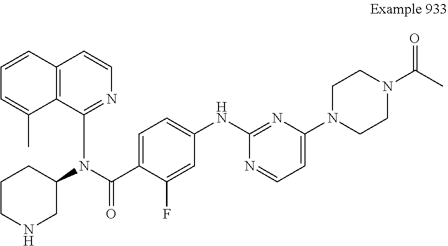 [SMILES: CC(=O)N1CCN(c2ccnc(Nc3ccc(C(=O)N(c4nccc5cccc(C)c45)[C@@H]4CCCNC4)c(F)c3)n2)CC1]

Example 933 was synthesized following the procedure for 684 by substituting (R)-pyrrolidin-3-ol with 1-(piperazin-1-yl)ethan-1-one to afford the title compound as a yellow solid. LC-MS: [M+H⁺]=583.3. 1H NMR (CD₃OD, 400 MHz): δ ppm 8.45-8.41 (m, 1H), 7.82-7.72 (m, 2H), 7.70-7.65 (m, 1H), 7.55-7.47 (m, 2H), 7.16-7.02 (m, 1H), 6.89-6.80 (m, 2H), 6.60 (d, J=6.8 Hz, 1H), 5.09-5.03 (m, 1H), 3.99-3.97 (m, 1H), 3.90-3.70 (m, 10H), 3.02-2.97 (m, 1H), 2.82 (s, 3H), 2.70-2.69 (m, 1H), 2.19-2.15 (m, 3H), 1.94-1.88 (m, 2H), 1.59-1.56 (m, 1H).

### Synthesis of (R)-2-fluoro-N-(8-methylisoquinolin-1-yl)-4-((4-((4-methylpiperazin-1-yl)methyl)pyrimidin-2-yl)amino)-N-(piperidin-3-yl)benzamide (Example 934)

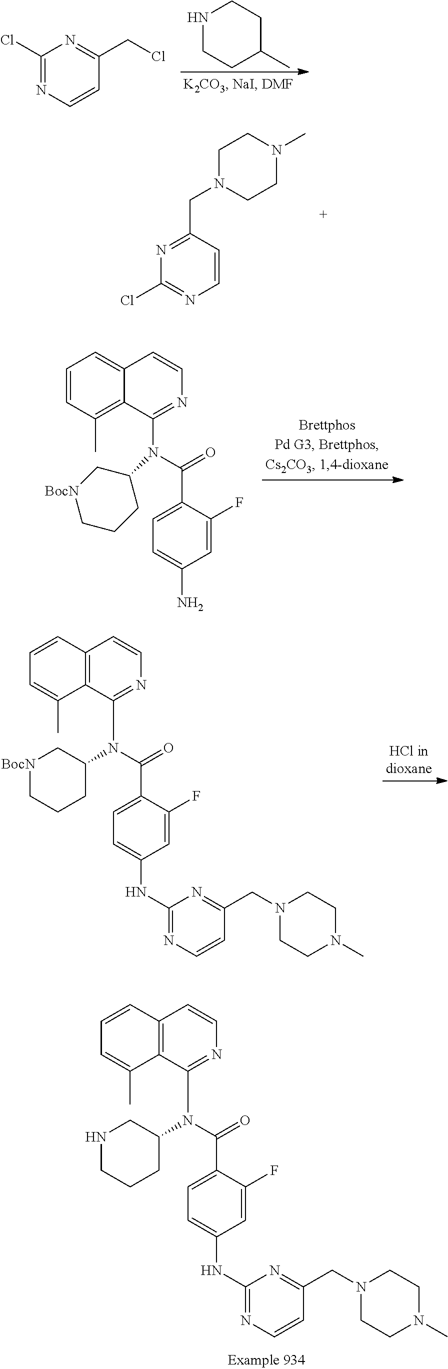

To a stirred mixture of 2-chloro-4-(chloromethyl)pyrimidine (500 mg, 3.06 mmol) and 1-methyl-piperazine (307 mg, 3.06 mmol) in DMF (10 mL) were added NaI (229 mg, 1.52 mmol) and K₂CO₃ (1.27 g, 9.18 mmol) in portions at 0° C. under nitrogen atmosphere. The resulting mixture was stirred for 3 h at 25° C. under nitrogen atmosphere before the mixture was poured into ice/water (100 mL) and extracted with EtOAc (3×100 mL). The combined organic layer was washed with brine, dried over anhydrous sodium sulfate and concentrated. The residue was purified by silica gel column chromatography, eluted with CH₂Cl₂/MeOH (5:1) to afford 2-chloro-4-[(4-methylpiperazin-1-yl)methyl]pyrimidine (120 mg, 17% yield) as a yellow solid.

### Steps 2-3:

Example 934 was prepared according to the procedure described for Example 210 by substituting 2-chloropyrimidine with 2-chloro-4-[(4-methylpiperazin-1-yl)methyl]pyrimidine, provided the title compound as a white solid. LC-MS (ES, m/z): 569 [M+H]⁺; ¹H-NMR (DMSO-d₆, 400 MHz) δ (ppm): 10.20-9.70 (m, 1H), 8.56-8.34 (m, 2H), 7.96-7.64 (m, 2H), 7.61-7.39 (m, 3H), 7.03-6.85 (m, 2H), 6.70-6.53 (m, 1H), 4.63-4.29 (m, 1H), 3.97-3.40 (m, 3H), 3.17-3.05 (m, 1H), 2.97-2.89 (m, 1H), 2.88-2.78 (m, 1H), 2.77-2.66 (m, 3H), 2.45-2.25 (m, 7H), 2.22-1.96 (m, 4H), 1.77-1.43 (m, 3H), 1.32-1.01 (m, 1H).

### Synthesis of (R)-4-((4-(1,1-dioxidothiomorpholino)pyrimidin-2-yl)amino)-2-fluoro-N-(8-methylisoquinolin-1-yl)-N-(piperidin-3-yl)benzamide (Example 935)

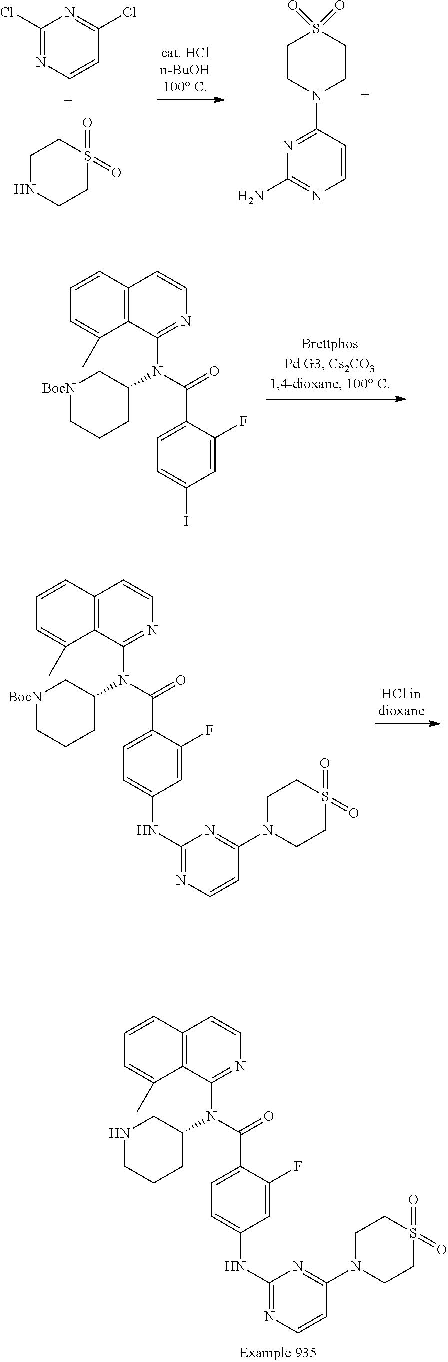 [SMILES: Clc1ccnc(Cl)n1 ; O=S1(=O)CCNCC1 ; Nc1nccc(N2CCS(=O)(=O)CC2)n1 ; Cc1cccc2ccnc(N(C(=O)c3ccc(I)cc3F)[C@@H]3CCCN(C(=O)OC(C)(C)C)C3)c12 ; Cc1cccc2ccnc(N(C(=O)c3ccc(Nc4nccc(N5CCS(=O)(=O)CC5)n4)cc3F)[C@@H]3CCCN(C(=O)OC(C)(C)C)C3)c12 ; Cc1cccc2ccnc(N(C(=O)c3ccc(Nc4nccc(N5CCS(=O)(=O)CC5)n4)cc3F)[C@@H]3CCCNC3)c12]

### Step 1:

To a stirred mixture of 4-chloropyrimidin-2-amine (1.00 g, 7.72 mmol) and thiomorpholine 1,1-dioxide (1.25 g, 9.26 mmol) in 1-butanol (20 mL) was added conc. HCl (10 drop) dropwise. The resulting mixture was stirred at 100° C. for 24 h under nitrogen atmosphere. The mixture was allowed to cool down to room temperature. The precipitated solid was collected by filtration and purified by silica gel column chromatography, eluted with CH₂Cl₂/MeOH (1:1) to afford 4-(2-aminopyrimidin-4-yl)-1lambda6-thiomorpholine-1,1-dione (700 mg, 40% yield) as an off-white solid.

### Steps 2-3:

Example 935 was prepared according to the procedure described for Example 684 by substituting (R)-1-(2-aminopyrimidin-4-yl)pyrrolidin-3-ol with 4-(2-aminopyrimidin-4-yl)-1lambda6-thiomorpholine-1,1-dione, provided the title compound as a white solid. LC-MS (ES, m/z): 590 [M+H]⁺; ¹H NMR (400 MHZ, DMSO-d₆) δ (ppm): 9.75-9.32 (m, 1H), 8.46-8.42 (m, 1H), 8.20-8.00 (m, 1H), 7.95-7.65 (m, 2H), 7.53-7.48 (m, 1H), 7.44-7.42 (m, 1H), 7.37-7.34 (m, 1H), 6.90-6.88 (m, 1H), 6.61-6.57 (m, 1H), 6.48-6.47 (m, 1H), 4.70-4.40 (m, 1H), 4.15-4.08 (m, 1H), 4.05-3.95 (m, 3H), 3.60-3.50 (m, 3H), 3.20-3.10 (m, 3H), 2.95-2.80 (m, 1H), 2.78-2.70 (m, 2H), 2.60-2.55 (m, 1H), 2.45-2.35 (m, 1H), 2.25-2.00 (m, 1H), 1.90-1.70 (m, 1H), 1.65-1.45 (m, 2H), 1.20-1.00 (m, 1H).

### Synthesis of (R)-2-fluoro-4-(4-(4-hydroxy-4-methylpiperidin-1-yl)pyrimidin-2-ylamino)-N-(8-methylisoquinolin-1-yl)-N-(piperidin-3-yl)benzamide (Example 936)

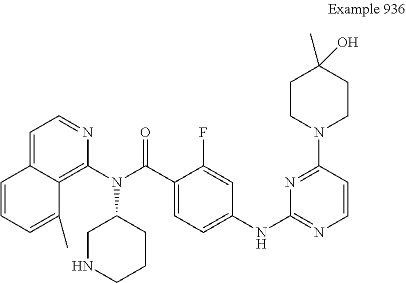 [SMILES: Cc1cccc2ccnc(N(C(=O)c3ccc(Nc4nccc(N5CCC(C)(O)CC5)n4)cc3F)[C@@H]3CCCNC3)c12]

Example 936 was prepared according to the procedure described for Example 684 by substituting (R)-pyrrolidin-3-ol with 4-methylpiperidin-4-ol, provided the title compound as a white solid. LC-MS (ES, m/z): 570 [M+H]⁺; ¹H NMR (400 MHz, DMSO-d₆) δ (ppm): 9.51-9.11 (m, 1H), 8.44-8.41 (m, 1H), 7.88-7.87 (m, 1H), 7.73-7.68 (m, 2H), 7.53-7.40 (m, 3H), 6.87-6.84 (m, 1H), 6.58-6.53 (m, 1H), 6.29-6.27 (m, 1H), 4.49-4.42 (m, 2H), 4.00-3.83 (m, 2H), 3.46-3.43 (m, 1H), 3.42-3.35 (m, 2H), 3.25-3.10 (m, 1H), 3.00-2.80 (m, 1H), 2.80-2.70 (m, 3H), 2.70-2.62 (m, 1H), 2.41-2.35 (m, 1H), 2.10-1.88 (m, 1H), 1.80-1.30 (m, 7H), 1.21-1.12 (m, 3H).

### Synthesis of (R)-2-fluoro-N-(8-methylisoquinolin-1-yl)-4-(4-(3-oxomorpholino)pyrimidin-2-ylamino)-N-(piperidin-3-yl)benzamide (Example 937)

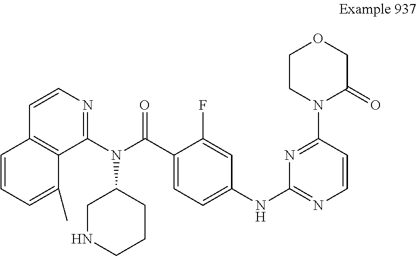 [SMILES: Cc1cccc2ccnc(N(C(=O)c3ccc(Nc4nccc(N5CCOCC5=O)n4)cc3F)[C@@H]3CCCNC3)c12]

Example 937 was prepared according to the procedure described for Example 935 by substituting thiomorpholine 1,1-dioxide with morpholin-3-one, provided the title compound as a white solid. LC-MS (ES, m/z): 556 [M+H]⁺; 1H-NMR (DMSO-d₆, 400 MHZ) δ ppm 10.13-9.72 (m, 1H), 8.45-8.42 (m, 1H), 8.40-8.36 (m, 1H), 7.92-7.65 (m, 3H), 7.55-7.43 (m, 2H), 7.34-7.31 (m, 1H), 6.95-6.93 (m, 1H), 6.67-6.63 (m, 1H), 4.49-4.33 (m, 1H), 4.28-4.21 (m, 2H), 4.06-3.99 (m, 2H), 3.88-3.85 (m, 1H), 3.50-3.47 (m, 1H), 3.18-3.06 (m, 1H), 2.99-2.93 (m, 1H), 2.86-2.83 (m, 1H), 2.76-2.64 (m, 3H), 2.60-2.57 (m, 1H), 2.47-2.33 (m, 1H), 2.20-1.84 (m, 1H), 1.80-1.39 (m, 2H), 1.38-1.02 (m, 1H).

### Synthesis of (R)-2-fluoro-N-(8-methylisoquinolin-1-yl)-4-((4-(3-oxopiperazin-1-yl)pyrimidin-2-yl)amino)-N-(piperidin-3-yl)benzamide (Example 938)

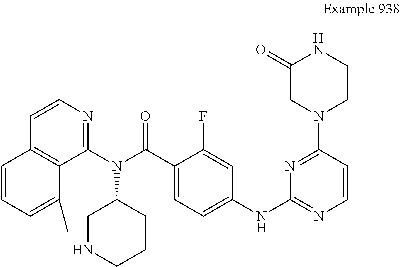 [SMILES: Cc1cccc2ccnc(N(C(=O)c3ccc(Nc4nccc(N5CCNC(=O)C5)n4)cc3F)[C@@H]3CCCNC3)c12]

Example 938 was prepared according to the procedure described for Example 935 by substituting thiomorpholine 1,1-dioxide with piperazin-2-one, provided the title compound as a white solid. LC-MS (ES, m/z): 555 [M+H]⁺; ¹H NMR (400 MHZ, DMSO-d₆) δ (ppm): 9.71-9.18 (m, 1H), 8.51-8.40 (m, 1H), 8.28-8.15 (m, 1H), 8.13-7.66 (m, 3H), 8.63-7.35 (m, 3H), 6.95-6.84 (m, 1H), 6.67-6.50 (m, 1H), 6.43-6.21 (m, 1H), 4.58-4.35 (m, 1H), 4.20-3.99 (m, 2H), 3.82-3.63 (m, 2H), 3.55-3.43 (m, 1H), 3.30-3.24 (m, 2H), 3.15-3.06 (m, 1H), 2.98-2.91 (m, 1H), 2.90-2.80 (m, 1H), 2.79-2.57 (m, 3H), 2.41-2.10 (m, 1H), 2.05-1.42 (m, 3H), 1.40-1.02 (m, 1H).

### Synthesis of 4-((4-((3R,5R)-3,5-dimethylmorpholino)pyrimidin-2-yl)amino)-2-fluoro-N-(8-methylisoquinolin-1-yl)-N—((R)-piperidin-3-yl)benzamide (Example 939)

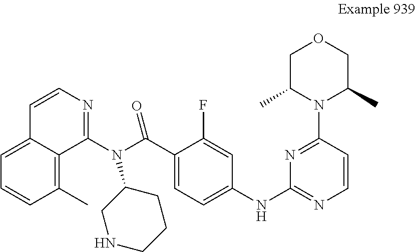 [SMILES: Cc1cccc2ccnc(N(C(=O)c3ccc(Nc4nccc(N5[C@H](C)COC[C@H]5C)n4)cc3F)[C@@H]3CCCNC3)c12]

Example 939 was prepared according to the procedure described for Example 935 by substituting thiomorpholine 1,1-dioxide with (3R,5R)-3,5-dimethylmorpholine, provided the title compound as a white solid. LC-MS (ESI, m/z): 570 [M+H]⁺; ¹H NMR (400 MHZ, DMSO-d₆) δ (ppm): 9.38-9.11 (m, 1H), 8.50-8.36 (m, 1H), 8.12-7.89 (m, 1H), 7.81-7.64 (m, 2H), 7.65-7.33 (m, 3H), 7.02-6.81 (m, 1H), 6.70-6.49 (m, 1H), 6.33-6.07 (m, 1H), 4.60-4.37 (m, 1H), 4.23-4.07 (m, 2H), 4.07-3.81 (m, 2H), 3.80-3.60 (m, 2H), 3.56-3.45 (m, 1H), 3.21-3.05 (m, 1H), 2.98-2.81 (m, 1H), 2.81-2.63 (m, 3H), 2.27-2.15 (m, 1H), 1.97-1.52 (m, 3H), 1.55-1.21 (m, 6H), 1.21-1.04 (m, 1H).

### Synthesis of 4-((4-((3R,5S)-3,5-dimethylmorpholino)pyrimidin-2-yl)amino)-2-fluoro-N-(8-methylisoquinolin-1-yl)-N—((R)-piperidin-3-yl)benzamide (Example 940)

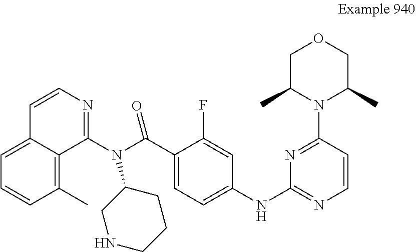 [SMILES: Cc1cccc2ccnc(N(C(=O)c3ccc(Nc4nccc(N5[C@H](C)COC[C@@H]5C)n4)cc3F)[C@@H]3CCCNC3)c12]

Example 940 was prepared according to the procedure described for Example 935 by substituting thiomorpholine 1,1-dioxide with (3R,5S)-3,5-dimethylmorpholine, provided the title compound as a white solid. LC-MS (ESI, m/z): 570 [M+H]⁺; ¹H NMR (400 MHZ, DMSO-d₆) δ (ppm): 9.19 (s, 1H), 8.53-8.36 (m, 1H), 8.13-7.86 (m, 1H), 7.84-7.65 (m, 2H), 7.65-7.48 (m, 1H), 7.47-7.30 (m, 2H), 6.95-6.82 (m, 1H), 6.64-6.48 (m, 1H), 6.33-6.13 (m, 1H), 4.58-4.37 (m, 1H), 4.10 (s, 2H), 3.88-3.72 (m, 2H), 3.69-3.39 (m, 3H), 3.21-3.03 (m, 1H), 3.04-2.56 (m, 5H), 2.26-2.11 (m, 1H), 2.05-1.43 (m, 3H), 1.39-1.31 (m, 1H), 1.28-0.43 (m, 6H).

### Synthesis of 2-fluoro-4-((4-((R)-3-isopropylmorpholino)pyrimidin-2-yl)amino)-N-(8-methylisoquinolin-1-yl)-N—((R)-piperidin-3-yl)benzamide (Example 941)

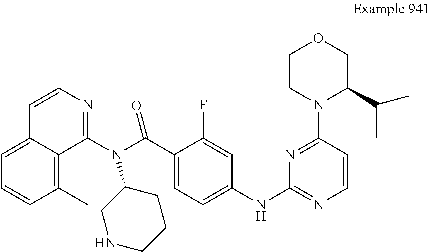 [SMILES: Cc1cccc2ccnc(N(C(=O)c3ccc(Nc4nccc(N5CCOC[C@H]5C(C)C)n4)cc3F)[C@@H]3CCCNC3)c12]

Example 941 was prepared according to the procedure described for Example 935 by substituting thiomorpholine 1,1-dioxide with (3R)-3-isopropylmorpholine, provided the title compound as a white solid. LC-MS (ESI, m/z): 584 [M+H]⁺. ¹H NMR (400 MHZ, DMSO-d₆) δ (ppm): 9.20-9.15 (m, 1H), 8.50-8.39 (m, 1H), 8.04-7.88 (m, 1H), 7.74-7.68 (m, 2H), 7.52-7.51 (m, 1H), 7.48-7.36 (m, 2H), 6.89-6.86 (m, 1H), 6.58-6.56 (m, 1H), 6.26-6.25 (m, 1H), 4.53-4.40 (m, 1H), 3.98-3.95 (m, 2H), 3.86-3.83 (m, 1H), 3.80-3.60 (m, 1H), 3.39-3.33 (m, 2H), 3.32-3.13 (m, 2H), 3.10-3.08 (m, 1H) 2.92-2.87 (m, 1H), 2.75-2.72 (m, 3H), 2.35-2.35 (m, 2H), 1.54-1.52 (m, 3H), 1.21-1.05 (m, 1H), 1.02-1.00 (m, 3H), 0.70-0.67 (m, 3H).

### Synthesis of 2-fluoro-4-((4-((R)-3-(hydroxymethyl)morpholino)pyrimidin-2-yl)amino)-N-(8-methylisoquinolin-1-yl)-N—((R)-piperidin-3-yl)benzamide (Example 942)

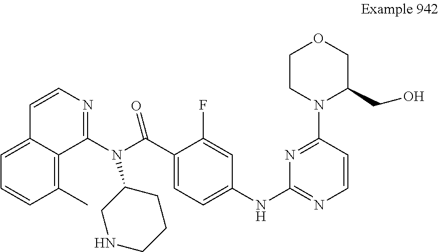 [SMILES: Cc1cccc2ccnc(N(C(=O)c3ccc(Nc4nccc(N5CCOC[C@H]5CO)n4)cc3F)[C@@H]3CCCNC3)c12]

Example 942 was prepared according to the procedure described for Example 935 by substituting thiomorpholine 1,1-dioxide with (3R)-morpholin-3-ylmethanol, provided the title compound as a white solid. LC-MS (ES, m/z): 572 [M+H]⁺; ¹H NMR (400 MHZ, DMSO-d₆) δ (ppm): 9.58-9.15 (m, 1H), 8.44-8.41 (m, 1H), 8.08-7.92 (m, 1H), 7.73-7.68 (m, 2H), 7.52-7.48 (m, 1H), 7.48-7.35 (m, 2H), 6.90-6.88 (m, 1H), 6.60-6.55 (m, 1H), 6.38-6.23 (m, 1H), 5.02-4.82 (m, 1H), 4.48-4.45 (m, 1H), 4.07-3.89 (m, 4H), 3.72-3.68 (m, 1H), 3.48-3.38 (m, 4H), 3.11-3.01 (m, 2H), 2.92-2.85 (m, 1H), 2.82-2.73 (m, 3H), 2.31-2.29 (m, 2H), 1.90-1.51 (m, 3H), 1.45-1.02 (m, 1H).

### Synthesis of 2-fluoro-4-(4-((R)-3-(methoxymethyl)morpholino)pyrimidin-2-ylamino)-N-(8-methylisoquinolin-1-yl)-N—((R)-piperidin-3-yl)benzamide (Example 943)

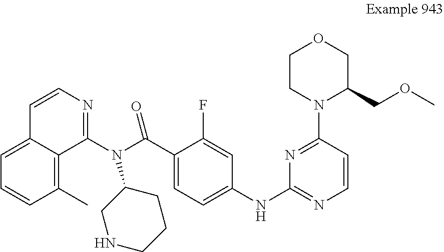 [SMILES: COC[C@@H]1COCCN1c1ccnc(Nc2ccc(C(=O)N(c3nccc4cccc(C)c34)[C@@H]3CCCNC3)c(F)c2)n1]

Example 943 was prepared according to the procedure described for Example 935 by substituting thiomorpholine 1,1-dioxide with (R)-3-(methoxymethyl)morpholine hydrochloride, provided the title compound as a white solid. LC-MS (ES, m/z): 586 [M+H]⁺; ¹H NMR (400 MHZ, DMSO-d₆) δ (ppm): 9.19 (s, 1H), 8.48-8.39 (m, 1H), 8.10-7.89 (m, 1H), 7.80-7.66 (m, 2H), 7.65-7.49 (m, 1H), 7.48-7.41 (m, 1H), 7.40-7.30 (m, 1H), 6.97-6.85 (m, 1H), 6.63-6.52 (m, 1H), 6.40-6.22 (m, 1H), 4.57-4.21 (m, 2H), 4.07-3.76 (m, 3H), 3.75-3.55 (m, 1H), 3.54-3.39 (m, 3H), 3.22 (s, 3H), 3.20-3.01 (m, 2H), 2.93 (s, 1H), 2.89-2.79 (m, 1H), 2.79-2.56 (m, 3H), 2.38-2.25 (m, 1H), 2.24-2.05 (m, 1H), 1.83-1.41 (m, 3H), 1.24-1.05 (m, 1H).

### Synthesis of (R)-4-(4-(1,4-oxazepan-4-yl)pyrimidin-2-ylamino)-2-fluoro-N-(8-methylisoquinolin-1-yl)-N-(piperidin-3-yl)benzamide (Example 944)

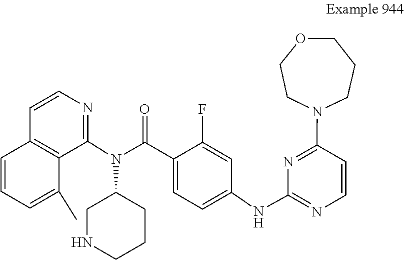 [SMILES: Cc1cccc2ccnc(N(C(=O)c3ccc(Nc4nccc(N5CCCOCC5)n4)cc3F)[C@@H]3CCCNC3)c12]

Example 944 was prepared according to the procedure described for Example 935 by substituting thiomorpholine 1,1-dioxide with 1,4-oxazepane, provided the title compound as a white solid. LC-MS (ES, m/z): 566 [M+H]⁺; ¹H NMR (400 MHZ, DMSO-d₆) δ ppm 9.62-9.12 (m, 1H), 8.51-8.38 (m, 1H), 8.09-7.35 (m, 6H), 6.96-6.81 (m, 1H), 6.63-6.49 (m, 1H), 6.44-5.98 (m, 1H), 4.55-4.37 (m, 1H), 3.81-3.74 (m, 1H), 3.74-3.66 (m, 1H), 3.65-3.58 (m, 3H), 3.57-3.51 (m, 2H), 3.51-3.42 (m, 1H), 3.16-3.01 (m, 1H), 2.98-2.89 (m, 1H), 2.87-2.65 (m, 4H), 2.35-2.28 (m, 1H), 2.27-2.10 (m, 1H), 1.99-1.64 (m, 3H), 1.61-1.25 (m, 3H), 1.21-1.01 (m, 1H).

### Synthesis of methyl(S)-4-(2-((3-fluoro-4-((8-methylisoquinolin-1-yl)((R)-piperidin-3-yl)carbamoyl)phenyl)amino)pyrimidin-4-yl)morpholine-3-carboxylate (Example 945)

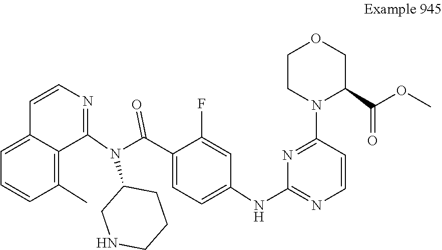 [SMILES: COC(=O)[C@@H]1COCCN1c1ccnc(Nc2ccc(C(=O)N(c3nccc4cccc(C)c34)[C@@H]3CCCNC3)c(F)c2)n1]

Example 945 was prepared according to the procedure described for Example 935 by substituting thiomorpholine 1,1-dioxide with methyl (3S)-morpholine-3-carboxylate, provided the title compound as a white solid. LC-MS (ES, m/z): 600 [M+H]⁺; ¹H-NMR (DMSO-d₆, 400 MHz) δ (ppm): δ 9.73-9.24 (m, 1H), 8.51-8.40 (m, 1H), 8.17-7.90 (m, 1H), 7.77-7.67 (m, 2H), 7.56-7.48 (m, 1H), 7.46-7.40 (m, 1H), 7.33-7.21 (m, 1H), 6.88-6.79 (m, 1H), 6.63-6.53 (m, 1H), 6.41-6.31 (m, 1H), 5.20-4.90 (m, 1H), 4.57-4.40 (m, 1H), 4.36-4.24 (m, 1H), 4.03-3.89 (m, 1H), 3.83-3.69 (m, 2H), 3.68-3.58 (m, 3H), 3.56-3.45 (m, 2H), 3.25-3.06 (m, 2H), 2.98-2.91 (m, 1H), 2.90-2.80 (m, 1H), 2.78-2.68 (m, 3H), 2.40-2.17 (m, 1H), 1.80-1.45 (m, 3H), 1.20-1.04 (m, 1H).

### Synthesis of 2-fluoro-4-(4-((1r,4r)-4-hydroxycyclohexylamino)pyrimidin-2-ylamino)-N-(8-methylisoquinolin-1-yl)-N—((R)-piperidin-3-yl)benzamide (Example 946)

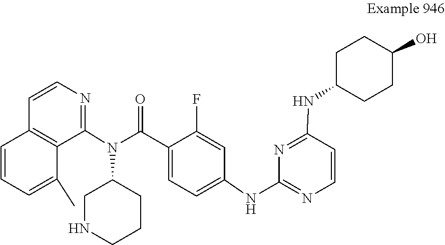 [SMILES: Cc1cccc2ccnc(N(C(=O)c3ccc(Nc4nccc(N[C@H]5CC[C@H](O)CC5)n4)cc3F)[C@@H]3CCCNC3)c12]

Example 946 was prepared according to the procedure described for Example 935 by substituting thiomorpholine 1,1-dioxide with trans-4-aminocyclohexanol, provided the title compound as a white solid. LC-MS (ES, m/z): 570 [M+H]⁺; ¹H NMR (400 MHZ, DMSO-d₆) δ (ppm): 9.52-9.10 (m, 1H), 8.46-8.43 (m, 1H), 8.15-7.69 (m, 3H), 7.53-7.48 (m, 2H), 7.44-7.42 (m, 1H), 7.20-7.10 (m, 1H), 6.90-6.89 (m, 1H), 6.56-6.52 (m, 1H), 6.00-5.88 (m, 1H), 4.69-4.64 (m, 1H), 4.55-4.45 (m, 1H), 3.95-3.55 (m, 1H), 3.49-3.44 (m, 2H), 3.14-3.08 (m, 1H), 2.93-2.83 (m, 2H), 2.75-2.68 (m, 3H), 2.38-2.33 (m, 1H), 2.20-2.10 (m, 1H), 2.00-1.70 (m, 4H), 1.60-1.50 (m, 2H), 1.35-1.10 (m, 5H).

### Synthesis of (R)-2-fluoro-N-(8-methylisoquinolin-1-yl)-4-((4-(3-oxopyrrolidin-1-yl)pyrimidin-2-yl)amino)-N-(piperidin-3-yl)benzamide (Example 947)

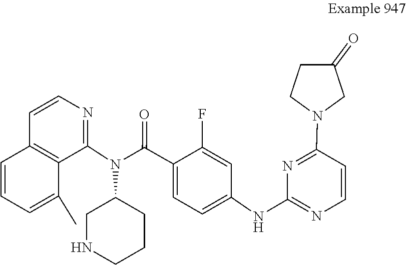 [SMILES: Cc1cccc2ccnc(N(C(=O)c3ccc(Nc4nccc(N5CCC(=O)C5)n4)cc3F)[C@@H]3CCCNC3)c12]

Example 947 was prepared according to the procedure described for Example 935 by substituting thiomorpholine 1,1-dioxide with 1,4-dioxa-7-azaspiro[4.4]nonane, provided the title compound as a white solid. LC-MS (ES, m/z): 540 [M+H]⁺. ¹H-NMR (CD₃OD, 400 MHz) δ: 8.67-8.51 (m, 1H), 8.50-8.39 (m, 1H), 7.99-7.81 (m, 1H), 7.79-7.63 (m, 2H), 7.62-7.40 (m, 2H), 6.92-6.73 (m, 1H), 6.70-6.56 (m, 1H), 6.20-5.89 (m, 1H), 5.14-5.02 (m, 1H), 4.60-3.72 (m, 4H), 3.70-3.52 (m, 1H), 3.51-3.42 (m, 1H), 3.41-3.36 (m, 1H), 3.28-2.90 (m, 2H), 2.89-2.63 (m, 3H), 2.30-1.80 (m, 3H), 1.68-1.50 (m, 1H).

### Synthesis of 4-({4-[cyclopentyl(methyl)amino]pyrimidin-2-yl}amino)-2-fluoro-N-(8-methylisoquinolin-1-yl)-N-[(3R)-piperidin-3-yl]benzamide (Example 948)

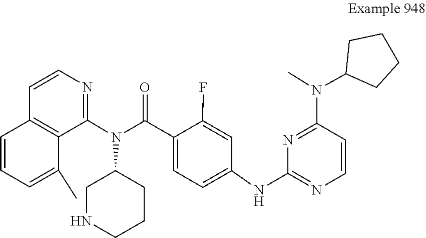 [SMILES: Cc1cccc2ccnc(N(C(=O)c3ccc(Nc4nccc(N(C)C5CCCC5)n4)cc3F)[C@@H]3CCCNC3)c12]

Example 948 was prepared according to the procedure described for Example 935 by substituting thiomorpholine 1,1-dioxide with N-methylcyclopentanamine, provided the title compound as a white solid. LC-MS (ES, m/z): 554 [M+H]⁺; ¹H NMR (400 MHZ, DMSO-d₆) δ (ppm): 9.58-9.10 (m, 1H), 8.50-8.40 (m, 1H), 8.15-7.66 (m, 3H), 7.65-7.41 (m, 3H), 6.87-6.85 (m, 1H), 6.57-6.53 (m, 1H), 6.30-6.14 (m, 1H), 5.09-4.61 (m, 1H), 4.51-4.28 (m, 1H), 4.01-3.41 (m, 1H), 3.16-3.07 (m, 1H), 2.99-2.91 (m, 1H), 2.88-2.75 (m, 3H), 2.74-2.69 (m, 3H), 2.38-2.16 (m, 1H), 1.88-1.72 (m, 4H), 1.70-1.22 (m, 7H), 1.19-1.05 (m, 1H).

### Synthesis of (R)-4-((4-((2-cyanoethyl)(methyl)amino)pyrimidin-2-yl)amino)-2-fluoro-N-(8-methylisoquinolin-1-yl)-N-(piperidin-3-yl)benzamide (Example 949)

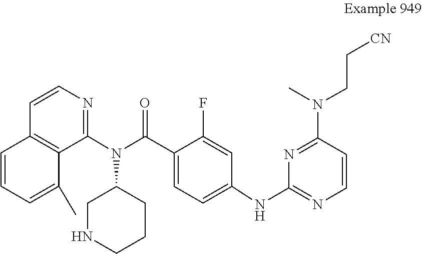 [SMILES: Cc1cccc2ccnc(N(C(=O)c3ccc(Nc4nccc(N(C)CCC#N)n4)cc3F)[C@@H]3CCCNC3)c12]

Example 949 was prepared according to the procedure described for Example 935 by substituting thiomorpholine 1,1-dioxide with 3-(methylamino) propanenitrile, provided the title compound as a white solid. LC-MS (ESI, m/z): 539 [M+H]⁺; ¹H NMR (400 MHZ, DMSO-d₆) δ (ppm): 9.52-9.21 (m, 1H), 8.48-8.41 (m, 1H), 8.19-7.91 (m, 1H), 7.71-7.68 (m, 2H), 7.52-7.50 (m, 1H), 7.49-7.38 (m, 2H), 6.89-6.88 (m, 1H), 6.58-6.56 (m, 1H), 6.26-6.22 (m, 1H), 4.55-4.40 (m, 1H), 3.78-3.75 (m, 2H), 3.48-3.46 (m, 1H), 3.13-3.07 (m, 1H), 3.02 (s, 2H), 3.01-2.95 (m, 1H), 2.93-2.86 (m, 2H), 2.77-2.67 (m, 5H), 2.33-2.30 (s, 1H), 2.20-2.17 (m, 1H), 1.81-1.43 (m, 2H), 1.30-1.03 (m, 1H).

### Synthesis of (R)-4-((4-((cyanomethyl)amino)pyrimidin-2-yl)amino)-2-fluoro-N-(8-methylisoquinolin-1-yl)-N-(piperidin-3-yl)benzamide (Example 950)

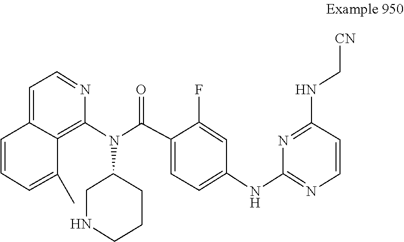 [SMILES: Cc1cccc2ccnc(N(C(=O)c3ccc(Nc4nccc(NCC#N)n4)cc3F)[C@@H]3CCCNC3)c12]

Example 950 was prepared according to the procedure described for Example 935 by substituting thiomorpholine 1,1-dioxide with aminoacetonitrile, provided the title compound as a white solid. LC-MS (ES, m/z): 511 [M+H]⁺; 1H-NMR (DMSO-d₆, 400 MHz) δ (ppm): 9.74-9.31 (m, 1H), 8.43-8.40 (m, 1H), 8.13-7.84 (m, 2H), 7.73-7.68 (m, 2H), 7.53-7.42 (m, 3H), 6.96-6.94 (m, 1H), 6.60-6.56 (m, 1H), 6.24-6.08 (m, 1H), 4.48-4.42 (m, 1H), 4.29-3.84 (m, 2H), 3.56-3.40 (m, 1H), 3.12-3.07 (m, 1H), 2.93-2.73 (m, 4H), 2.64-2.59 (m, 1H), 2.33-2.24 (m, 1H), 2.23-1.67 (m, 1H), 1.61-1.40 (m, 2H), 1.38-1.10 (m, 1H).

### Synthesis of 2-fluoro-4-[(4-methoxypyrimidin-2-yl)amino]-N-(8-methylisoquinolin-1-yl)-N-[(3R)-piperidin-3-yl]benzamide (Example 951)

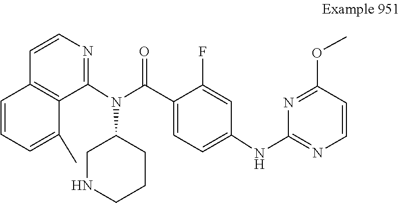 [SMILES: COc1ccnc(Nc2ccc(C(=O)N(c3nccc4cccc(C)c34)[C@@H]3CCCNC3)c(F)c2)n1]

Example 951 was prepared according to the procedure described for Example 684 by substituting (R)-1-(2-aminopyrimidin-4-yl)pyrrolidin-3-ol with 2-amino-4-methoxy-pyrimidine, provided the title compound as a white solid. LC-MS (ES, m/z): 487 [M+H]⁺; ¹H NMR (400 MHZ, DMSO-d₆) δ (ppm): 10.10-10.51 (m, 1H), 8.53-8.38 (m, 1H), 8.34-7.94 (m, 1H), 7.94-7.65 (m, 2H), 7.65-7.42 (m, 3H), 7.01-6.88 (m, 1H), 6.72-6.55 (m, 1H), 6.47-6.28 (m, 1H), 4.58-4.39 (m, 1H), 4.01-3.76 (m, 3H), 3.55-3.42 (m, 1H), 3.18-3.03 (m, 1H), 2.96-2.76 (m, 2H), 2.75-2.66 (m, 2H), 2.62-2.51 (m, 1H), 2.38-2.26 (m, 1H), 1.75-1.26 (m, 3H), 1.22-0.99 (m, 1H).

### Synthesis of 4-((4-(sec-butyl(methyl)amino)pyrimidin-2-yl)amino)-2-fluoro-N-(8-methylisoquinolin-1-yl)-N—((R)-piperidin-3-yl)benzamide (Example 952)

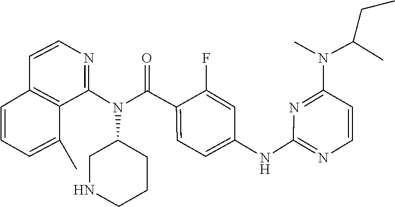 [SMILES: CCC(C)N(C)c1ccnc(Nc2ccc(C(=O)N(c3nccc4cccc(C)c34)[C@@H]3CCCNC3)c(F)c2)n1]

Example 952 was prepared according to the procedure described for Example 684 by substituting (R)-pyrrolidin-3-ol with N-methylbutan-2-amine, provided the title compound as a white solid. LC-MS (ES, m/z): 542 [M+H]⁺; ¹H NMR (400 MHZ, DMSO-d₆) δ (ppm): 9.58-9.07 (m, 1H), 8.50-8.39 (m, 1H), 8.09-7.65 (m, 3H), 7.60-7.40 (m, 3H), 6.91-6.81 (m, 1H), 6.61-6.50 (m, 1H), 6.29-6.02 (m, 1H), 4.92-4.40 (m, 1H), 3.99-3.42 (m, 1H), 3.23-3.00 (m, 1H), 2.95-2.53 (m, 8H), 2.40-1.88 (m, 2H), 1.87-1.20 (m, 5H), 1.18-1.10 (m, 1H), 1.09-1.01 (m, 3H), 0.89-0.68 (m, 3H).

### Synthesis of (R)-4-(1,2,4-triazin-3-ylamino)-2-fluoro-N-(8-methylisoquinolin-1-yl)-N-(piperidin-3-yl)benzamide (Example 953)

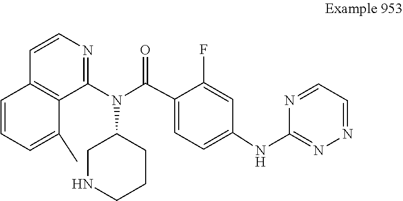 [SMILES: Cc1cccc2ccnc(N(C(=O)c3ccc(Nc4nccnn4)cc3F)[C@@H]3CCCNC3)c12]

Example 953 was prepared according to the procedure described for Example 684 by substituting (R)-1-(2-aminopyrimidin-4-yl)pyrrolidin-3-ol with 3-amino-1,2,4-triazine, provided the title compound as a white solid. LC-MS (ES, m/z): 458 [M+H⁺]; ¹H NMR (400 MHz, DMSO-d₆) δ (ppm): 10.81-9.90 (m, 1H), 9.00-8.79 (m, 1H), 8.64-8.26 (m, 2H), 8.02-7.39 (m, 5H), 7.19-6.87 (m, 1H), 6.82-6.42 (m, 1H), 4.62-4.31 (m, 1H), 4.02-4.39 (m, 1H), 3.15-3.04 (m, 1H), 3.00-2.89 (m, 1H), 2.88-2.66 (m, 4H), 2.38-2.24 (m, 1H), 2.20-1.85 (m, 1H), 1.81-1.67 (m, 1H), 1.65-1.44 (m, 2H), 1.39-0.95 (m, 1H).

### Synthesis of (R)-2-fluoro-N-(8-methylisoquinolin-1-yl)-N-(piperidin-3-yl)-4-((4-((tetrahydro-2H-pyran-4-yl)amino)pyrimidin-2-yl)amino)benzamide (Example 954)

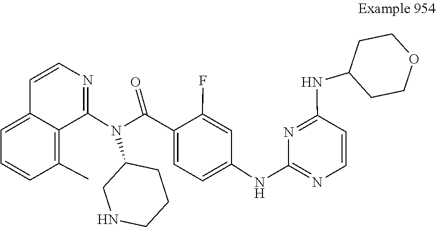 [SMILES: Cc1cccc2ccnc(N(C(=O)c3ccc(Nc4nccc(NC5CCOCC5)n4)cc3F)[C@@H]3CCCNC3)c12]

Example 954 was prepared according to the procedure described for Example 935 by substituting thiomorpholine 1,1-dioxide with tetrahydro-2H-pyran-4-amine, provided the title compound as a white solid. LC-MS (ES, m/z): 556 [M+H]⁺; ¹H NMR (400 MHZ, DMSO-d₆) δ (ppm): 9.55-9.12 (m, 1H), 8.43-8.40 (m, 1H), 8.12-7.68 (m, 3H), 7.54-7.48 (m, 2H), 7.43-7.41 (m, 1H), 7.36-7.26 (m, 1H), 6.88-6.85 (m, 1H), 6.56-6.52 (m, 1H), 6.05-5.92 (m, 1H), 4.52-4.40 (m, 1H), 4.10-3.80 (m, 3H), 3.47-3.44 (m, 1H), 3.38-3.35 (m, 2H), 3.08-2.81 (m, 2H), 2.75-2.72 (m, 3H), 2.49-2.10 (m, 2H), 2.00-1.73 (m, 2H), 1.60-1.35 (m, 4H), 1.18-1.02 (m, 1H).

### Synthesis of 4-((4-(3-cyanomorpholino)pyrimidin-2-yl)amino)-2-fluoro-N-(8-methylisoquinolin-1-yl)-N—((R)-piperidin-3-yl)benzamide (Example 955)

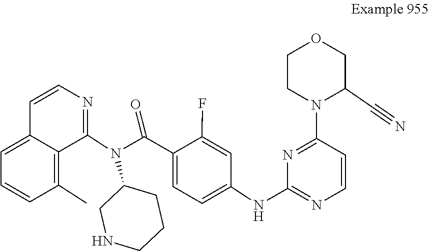 [SMILES: Cc1cccc2ccnc(N(C(=O)c3ccc(Nc4nccc(N5CCOCC5C#N)n4)cc3F)[C@@H]3CCCNC3)c12]

Example 955 was prepared according to the procedure described for Example 935 by substituting thiomorpholine 1,1-dioxide with morpholine-3-carbonitrile, provided the title compound as an off-white solid. LC-MS (ESI, m/z): 567 [M+H⁺]; ¹H NMR (400 MHZ, DMSO-d₆) δ (ppm): 9.94-9.36 (m, 1H), 8.59-8.37 (m, 1H), 8.38-8.11 (m, 1H), 8.04-7.66 (m, 2H), 7.67-7.40 (m, 2H), 7.39-7.23 (m, 1H), 7.02-6.82 (m, 1H), 6.70-6.55 (m, 1H), 6.53-6.43 (m, 1H), 5.56-5.30 (m, 1H), 4.59-4.31 (m, 1H), 4.29-4.13 (m, 1H), 4.13-3.84 (m, 2H), 3.84-3.67 (m, 1H), 3.67-3.43 (m, 1H), 3.19-3.00 (m, 1H), 3.00-2.80 (m, 1H), 2.77 (s, 1H), 2.74 (s, 1H), 2.38-2.16 (m, 1H), 2.09-1.40 (m, 3H), 1.34-0.98 (m, 1H).

### Synthesis of 4-(4-(3-((dimethylamino)methyl)morpholino)pyrimidin-2-ylamino)-2-fluoro-N-(8-methylisoquinolin-1-yl)-N—((R)-piperidin-3-yl)benzamide (Example 956)

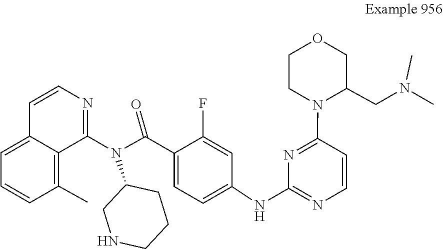 [SMILES: Cc1cccc2ccnc(N(C(=O)c3ccc(Nc4nccc(N5CCOCC5CN(C)C)n4)cc3F)[C@@H]3CCCNC3)c12]

Example 956 was prepared according to the procedure described for Example 684 by substituting (R)-pyrrolidin-3-ol with N,N-dimethyl-1-(morpholin-3-yl)methanamine, provided the title compound as a white solid. LC-MS (ES, m/z): 599 [M+H⁺]; ¹H NMR (400 MHz, DMSO-d₆) δ (ppm): 9.17-9.12 (m, 1H), 8.50-8.39 (m, 1H), 7.96-7.89 (m, 2H), 7.76-7.67 (m, 1H), 7.58-7.33 (m, 3H), 6.94-6.85 (m, 1H), 6.64-6.52 (m, 1H), 6.25-6.19 (m, 1H), 4.54-4.40 (m, 1H), 4.26 (s, 1H), 4.11-3.80 (m, 3H), 3.49-3.34 (m, 3H), 3.15-3.01 (m, 2H), 2.96-2.90 (m, 1H), 2.88-2.81 (m, 1H), 2.78-2.68 (m, 4H), 2.46-2.26 (m, 1H), 2.24-2.19 (m, 1H), 2.16-2.08 (m, 6H), 1.90-1.21 (m, 3H), 1.11 (s, 1H).

### Synthesis of (R)-2-fluoro-4-(4-(methyl(tetrahydro-2H-pyran-4-yl)amino)pyrimidin-2-ylamino)-N-(8-methylisoquinolin-1-yl)-N-(piperidin-3-yl)benzamide (Example 957)

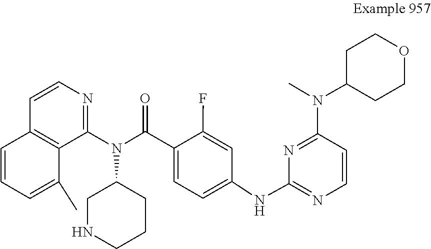 [SMILES: Cc1cccc2ccnc(N(C(=O)c3ccc(Nc4nccc(N(C)C5CCOCC5)n4)cc3F)[C@@H]3CCCNC3)c12]

Example 957 was prepared according to the procedure described for Example 684 by substituting (R)-pyrrolidin-3-ol with N-methyltetrahydro-2H-pyran-4-amine, provided the title compound as a white solid. LC-MS (ES, m/z): 570 [M+H⁺]. ¹H NMR (400 MHZ, DMSO-d₆) δ (ppm): 9.62-9.20 (m, 1H), 8.43-8.40 (m, 1H), 8.05-7.89 (m, 1H), 7.74-7.68 (m, 2H), 7.52-7.41 (m, 3H), 6.88-6.85 (m, 1H), 6.59-6.55 (m, 1H), 6.20-6.16 (m, 1H), 4.65-4.40 (m, 2H), 3.99-3.90 (m, 2H), 3.50-3.35 (m, 3H), 3.15-3.12 (m, 1H), 2.92-2.82 (m, 4H), 2.75-2.73 (m, 3H), 2.37-2.25 (m, 1H), 2.00-1.72 (m, 3H), 1.65-1.45 (m, 4H), 1.20-1.00 (m, 1H).

### Synthesis of 2-fluoro-N-(8-methylisoquinolin-1-yl)-N—((R)-piperidin-3-yl)-4-((4-(3-(pyridin-3-yl)morpholino)pyrimidin-2-yl)amino)benzamide (Example 958)

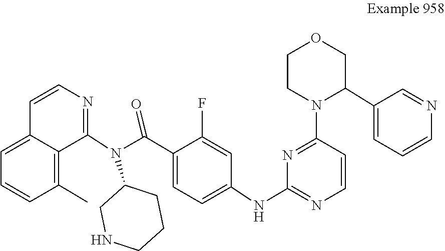 [SMILES: Cc1cccc2ccnc(N(C(=O)c3ccc(Nc4nccc(N5CCOCC5c5cccnc5)n4)cc3F)[C@@H]3CCCNC3)c12]

Example 958 was prepared according to the procedure described for Example 684 by substituting (R)-pyrrolidin-3-ol with 3-(pyridin-3-yl)morpholine dihydrochloride, provided the title compound as a white solid. LC-MS (ES, m/z): 619 [M+H]⁺; ¹H NMR (400 MHZ, DMSO-d₆) δ (ppm): 9.69-9.13 (m, 1H), 8.68-8.45 (m, 2H), 8.44-8.10 (m, 1H), 8.03-7.90 (m, 1H), 7.89-7.62 (m, 3H), 7.61-7.15 (m, 4H), 6.90-6.72 (m, 1H), 6.61-6.50 (m, 1H), 6.49-6.33 (m, 1H), 5.68-5.47 (m, 1H), 4.56-4.10 (m, 2H), 4.09-3.67 (m, 3H), 3.65-3.42 (m, 1H), 3.28-3.02 (m, 2H), 2.98-2.80 (m, 1H), 2.79-2.65 (m, 3H), 2.63-2.52 (m, 1H), 2.49-2.42 (m, 1H), 2.41-2.24 (m, 1H), 2.22-1.62 (m, 1H), 1.61-1.40 (m, 2H), 1.38-1.01 (m, 1H).

### Synthesis of 2-fluoro-N-(8-methylisoquinolin-1-yl)-4-({4-[(2R)-2-methylpiperidin-1-yl]pyrimidin-2-yl}amino)-N-[(3R)-piperidin-3-yl]benzamide (Example 959)

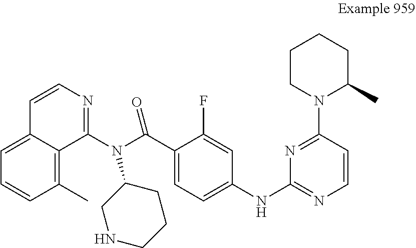 [SMILES: Cc1cccc2ccnc(N(C(=O)c3ccc(Nc4nccc(N5CCCC[C@H]5C)n4)cc3F)[C@@H]3CCCNC3)c12]

Example 959 was prepared according to the procedure described for Example 684 by substituting (R)-pyrrolidin-3-ol with (R)-2-methylpiperidine, provided the title compound as a white solid. LC-MS (ES, m/z): 554 [M+H]⁺; ¹H NMR (400 MHZ, DMSO-d₆) δ (ppm): 9.55-9.09 (m, 1H), 8.51-8.41 (m, 1H), 8.10-7.65 (m, 3H), 7.64-7.32 (m, 3H), 6.90-6.85 (m, 1H), 6.65-6.50 (m, 1H), 6.38-6.21 (m, 1H), 4.81-4.31 (m, 2H), 4.30-3.89 (m, 1H), 3.52-3.46 (m, 1H), 3.19-3.08 (m, 1H), 3.01-2.80 (m, 3H), 2.79-2.65 (m, 3H), 2.41-1.95 (m, 2H), 1.88-1.64 (m, 2H), 1.63-1.50 (m, 5H), 1.39-1.28 (m, 1H), 1.21-1.18 (m, 1H), 1.17-1.04 (m, 3H).

### Synthesis of 2-fluoro-4-((4-((S)-2-(hydroxymethyl)piperidin-1-yl)pyrimidin-2-yl)amino)-N-(8-methylisoquinolin-1-yl)-N—((R)-piperidin-3-yl)benzamide (Example 960)

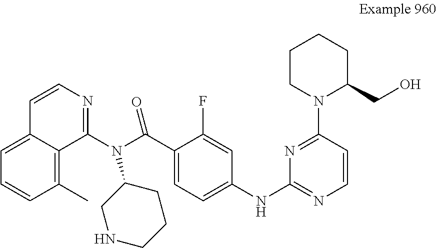 [SMILES: Cc1cccc2ccnc(N(C(=O)c3ccc(Nc4nccc(N5CCCC[C@H]5CO)n4)cc3F)[C@@H]3CCCNC3)c12]

Example 960 was prepared according to the procedure described for Example 684 by substituting (R)-pyrrolidin-3-ol with(S)-piperidin-2-ylmethanol, provided the title compound as a white solid. LC-MS (ES, m/z): 570 [M+H]⁺; 1H-NMR (DMSO-d₆, 400 MHZ) δ (ppm): δ 9.50-9.08 (m, 1H), 8.50-8.41 (m, 1H), 8.00-7.85 (m, 1H), 7.80-7.65 (m, 2H), 7.62-7.49 (m, 1H), 7.48-7.35 (m, 2H), 6.95-6.85 (m, 1H), 6.62-6.52 (m, 1H), 6.39-6.20 (m, 1H), 4.80-4.65 (m, 1H), 4.53-4.40 (m, 1H), 4.39-4.10 (m, 2H), 3.68-3.45 (m, 3H), 3.15-3.05 (m, 1H), 2.98-2.90 (m, 1H), 2.89-2.80 (m, 2H), 2.79-2.68 (m, 3H), 2.38-2.25 (m, 1H), 2.21-2.11 (m, 1H), 1.95-1.80 (m, 1H), 1.79-1.41 (m, 6H), 1.39-1.05 (m, 2H).

### Synthesis of (R)-2-fluoro-N-(8-methylisoquinolin-1-yl)-N-(piperidin-3-yl)-4-((4-(pyrrolidin-1-ylmethyl)pyrimidin-2-yl)amino)benzamide (Example 961)

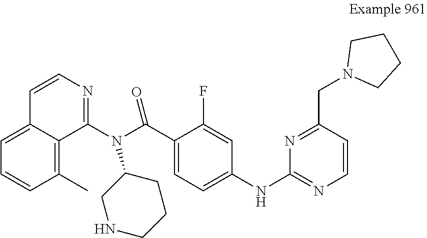 [SMILES: Cc1cccc2ccnc(N(C(=O)c3ccc(Nc4nccc(CN5CCCC5)n4)cc3F)[C@@H]3CCCNC3)c12]

Example 961 was prepared according to the procedure described for Example 934 by substituting 1-methyl-piperazine with pyrrolidine, provided the title compound as an off-white solid. LC-MS (ES, m/z): 540 [M+H]⁺; ¹H NMR (400 MHZ, DMSO-d₆) δ (ppm): 9.85-9.76 (m, 1H), 9.40-8.75 (m, 2H), 8.58-8.37 (m, 2H), 8.00-7.79 (m, 1H), 7.78-7.66 (m, 1H), 7.61-7.50 (m, 2H), 7.49-7.41 (m, 1H), 7.09-6.86 (m, 2H), 6.68-6.55 (m, 1H), 5.01-4.69 (m, 1H), 3.80-3.70 (m, 1H), 3.69-3.61 (m, 2H), 3.59-3.49 (m, 1H), 3.28-3.20 (m, 1H), 2.95-2.71 (m, 4H), 2.66-2.53 (m, 3H), 1.89-1.61 (m, 7H), 1.38-1.18 (m, 1H).

### Synthesis of (R)-2-fluoro-N-(8-methylisoquinolin-1-yl)-N-(piperidin-3-yl)-4-((4-(4-(pyridin-4-yl)piperidin-1-yl)pyrimidin-2-yl)amino)benzamide (Example 962)

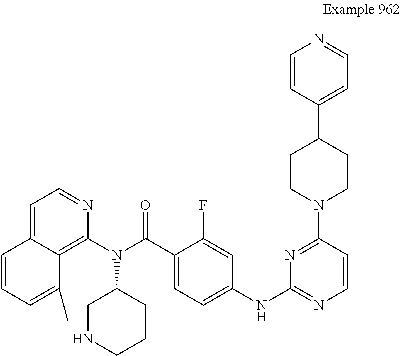 [SMILES: Cc1cccc2ccnc(N(C(=O)c3ccc(Nc4nccc(N5CCC(c6ccncc6)CC5)n4)cc3F)[C@@H]3CCCNC3)c12]

Example 962 was prepared according to the procedure described for Example 684 by substituting (R)-pyrrolidin-3-ol with 4-(piperidin-4-yl)pyridine, provided the title compound as a white solid. LC-MS (ESI, m/z): 617 [M+H]⁺. ¹H NMR (400 MHZ, DMSO-d₆) δ (ppm): 9.16 (s, 1H), 8.55-8.46 (m, 2H), 8.45-8.36 (m, 1H), 8.10-7.87 (m, 1H), 7.78-7.59 (m, 2H), 7.58-7.38 (m, 3H), 7.37-7.24 (m, 2H), 6.96-6.82 (m, 1H), 6.65-6.52 (m, 1H), 6.50-6.28 (m, 1H), 4.68-4.32 (m, 3H), 3.54-3.43 (m, 1H), 3.18-3.02 (m, 1H), 3.02-2.81 (m, 4H), 2.79-2.64 (m, 3H), 2.41-2.26 (m, 1H), 2.25-2.07 (m, 1H), 1.99-1.81 (m, 2H), 1.68-1.43 (m, 4H), 1.37-1.01 (m, 1H).

### Synthesis of 2-fluoro-4-({4-[4-(hydroxymethyl)-4-methylpiperidin-1-yl]pyrimidin-2-yl}amino)-N-(8-methylisoquinolin-1-yl)-N-[(3R)-piperidin-3-yl]benzamide (Example 963)

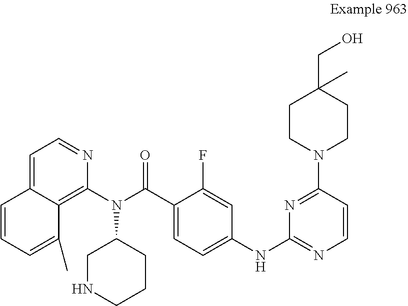 [SMILES: Cc1cccc2ccnc(N(C(=O)c3ccc(Nc4nccc(N5CCC(C)(CO)CC5)n4)cc3F)[C@@H]3CCCNC3)c12]

Example 963 was prepared according to the procedure described for Example 684 by substituting (R)-pyrrolidin-3-ol with (4-methylpiperidin-4-yl) methanol, provided the title compound as a white solid. LC-MS (ES, m/z): 584 [M+H]⁺; ¹H-NMR (DMSO-d₆, 400 MHZ) δ (ppm): δ 9.60-9.05 (m, 1H), 8.58-8.30 (m, 1H), 8.07-7.84 (m, 1H), 7.82-7.68 (m, 2H), 7.66-7.34 (m, 3H), 6.93-6.80 (m, 1H), 6.63-6.49 (m, 1H), 6.41-6.22 (m, 1H), 4.69-4.39 (m, 2H), 4.01-3.70 (m, 2H), 3.55-3.40 (m, 1H), 3.30-3.26 (m, 1H), 3.22-3.05 (m, 3H), 2.98-2.67 (m, 5H), 2.46-2.12 (m, 2H), 1.80-1.34 (m, 5H), 1.31-1.02 (m, 3H), 1.01-0.90 (m, 3H).

### Synthesis of (R)-2-fluoro-4-((4-(4-(methoxymethyl)piperidin-1-yl)pyrimidin-2-yl)amino)-N-(8-methylisoquinolin-1-yl)-N-(piperidin-3-yl)benzamide (Example 964)

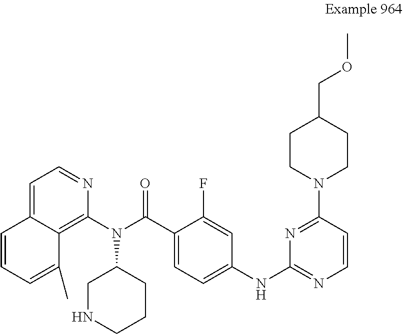 [SMILES: COCC1CCN(c2ccnc(Nc3ccc(C(=O)N(c4nccc5cccc(C)c45)[C@@H]4CCCNC4)c(F)c3)n2)CC1]

Example 964 was prepared according to the procedure described for Example 684 by substituting (R)-pyrrolidin-3-ol with 4-(methoxymethyl)piperidine, provided the title compound as a white solid. LC-MS (ESI, m/z): 584 [M+H]⁺; ¹H NMR (400 MHZ, DMSO-d₆) (ppm): 9.61-9.09 (m, 1H), 8.55-8.36 (m, 1H), 8.09-7.82 (m, 1H), 7.80-7.61 (m, 2H), 7.60-7.48 (m, 1H), 7.51-7.19 (m, 2H), 6.98-6.74 (m, 1H), 6.65-6.50 (m, 1H), 6.44-6.19 (m, 1H), 4.55-4.36 (m, 1H), 4.34-3.85 (m, 2H), 3.55-3.42 (m, 1H), 3.29-3.16 (m, 3H), 3.15-2.86 (m, 2H), 2.86-2.79 (m, 1H), 2.76 (s, 1H), 2.73 (s, 3H), 2.30 (s, 1H), 2.24-1.99 (m, 1H), 1.96-1.80 (m, 1H), 1.80-1.64 (m, 2H), 1.52 (s, 2H), 1.40-0.78 (m, 3H).

### Synthesis of methyl (2-((3-fluoro-4-((8-methylisoquinolin-1-yl)((R)-piperidin-3-yl)carbamoyl)phenyl)amino)pyrimidin-4-yl)-D-prolinate (Example 965)

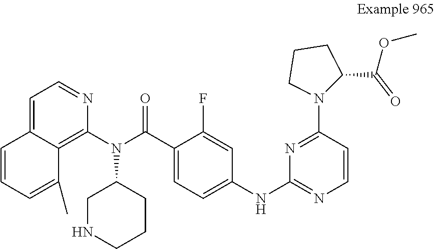 [SMILES: COC(=O)[C@H]1CCCN1c1ccnc(Nc2ccc(C(=O)N(c3nccc4cccc(C)c34)[C@@H]3CCCNC3)c(F)c2)n1]

Example 965 was prepared according to the procedure described for Example 684 by substituting (R)-pyrrolidin-3-ol with (2R)-pyrrolidine-2-carboxylate, provided the title compound as a white solid. LC-MS (ES, m/z): 584 [M+H]⁺; ¹H NMR (400 MHZ, DMSO-d₆) δ (ppm): 9.71-9.15 (m, 1H), 8.52-8.40 (m, 1H), 8.09-7.63 (m, 3H), 7.61-7.28 (m, 3H), 6.97-6.72 (m, 1H), 6.61-6.48 (m, 1H), 6.19-5.99 (m, 1H), 4.81-4.38 (m, 2H), 4.00-3.61 (m, 1H), 3.60-3.52 (m, 1H), 3.51-3.41 (m, 1H), 3.40-3.36 (m, 2H), 3.17-3.01 (m, 1H), 2.99-2.78 (m, 2H), 2.77-2.55 (m, 3H), 2.49-2.40 (m, 1H), 2.39-2.22 (m, 2H), 2.21-1.64 (m, 4H), 1.63-1.41 (m, 2H), 1.40-1.01 (m, 1H).

### Synthesis of 2-fluoro-4-({4-[(2R)-2-(hydroxymethyl)pyrrolidin-1-yl]pyrimidin-2-yl}amino)-N-(8-methylisoquinolin-1-yl)-N-[(3R)-piperidin-3-yl]benzamide (Example 966)

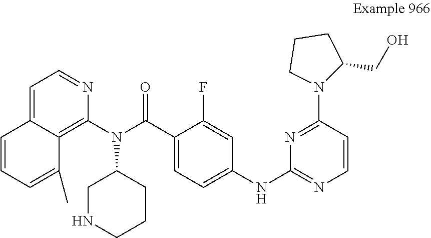 [SMILES: Cc1cccc2ccnc(N(C(=O)c3ccc(Nc4nccc(N5CCC[C@@H]5CO)n4)cc3F)[C@@H]3CCCNC3)c12]

Example 966 was prepared according to the procedure described for Example 684 by substituting (R)-pyrrolidin-3-ol with prolinol, provided the title compound as a white solid. LC-MS (ES, m/z): 556 [M+H]⁺. ¹H-NMR (DMSO-d₆, 400 MHZ) δ (ppm): δ (ppm): 9.63-8.91 (m, 1H), 8.50-8.36 (m, 1H), 8.00-7.82 (m, 1H), 7.80-7.62 (m, 2H), 7.58-7.41 (m, 3H), 6.99-6.80 (m, 1H), 6.64-6.50 (m, 1H), 6.27-5.79 (m, 1H), 4.93-3.65 (m, 3H), 3.53-3.42 (m, 2H), 3.13-3.02 (m, 1H), 2.98-2.80 (m, 2H), 2.78-2.69 (m, 3H), 2.61-2.56 (m, 1H), 2.36-1.83 (m, 7H), 1.78-1.42 (m, 3H), 1.31-1.01 (m, 1H).

### Synthesis of 4-({4-[(3S)-3-(dimethylamino)pyrrolidin-1-yl]pyrimidin-2-yl}amino)-2-fluoro-N-(8-methylisoquinolin-1-yl)-N-[(3R)-piperidin-3-yl]benzamide (Example 967)

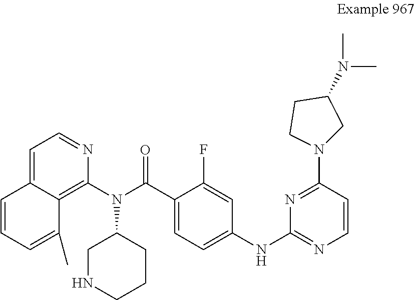 [SMILES: Cc1cccc2ccnc(N(C(=O)c3ccc(Nc4nccc(N5CC[C@H](N(C)C)C5)n4)cc3F)[C@@H]3CCCNC3)c12]

Example 967 was prepared according to the procedure described for Example 684 by substituting (R)-pyrrolidin-3-ol with (3S)—N,N-dimethylpyrrolidin-3-amine, provided the title compound as a white solid. LC-MS (ES, m/z): 569 [M+H]⁺; 1H-NMR (DMSO-d₆, 400 MHz) δ (ppm): δ 9.69-9.09 (m, 1H), 8.50-8.39 (m, 1H), 8.24-7.81 (m, 1H), 7.79-7.65 (m, 2H), 7.64-7.40 (m, 3H), 6.99-6.80 (m, 1H), 6.60-6.49 (m, 1H), 6.13-5.88 (m, 1H), 4.59-4.28 (m, 1H), 4.00-3.41 (m, 3H), 3.20-2.91 (m, 2H), 2.89-2.70 (m, 5H), 2.30-2.09 (m, 9H), 2.07-1.65 (m, 2H), 1.63-1.43 (m, 2H), 1.39-0.72 (m, 2H).

### Synthesis of 2-fluoro-N-(8-methylisoquinolin-1-yl)-N-[(3R)-piperidin-3-yl]-4-{[4-(pyrrolidin-1-yl)-1,3,5-triazin-2-yl]amino}benzamide (Example 968)

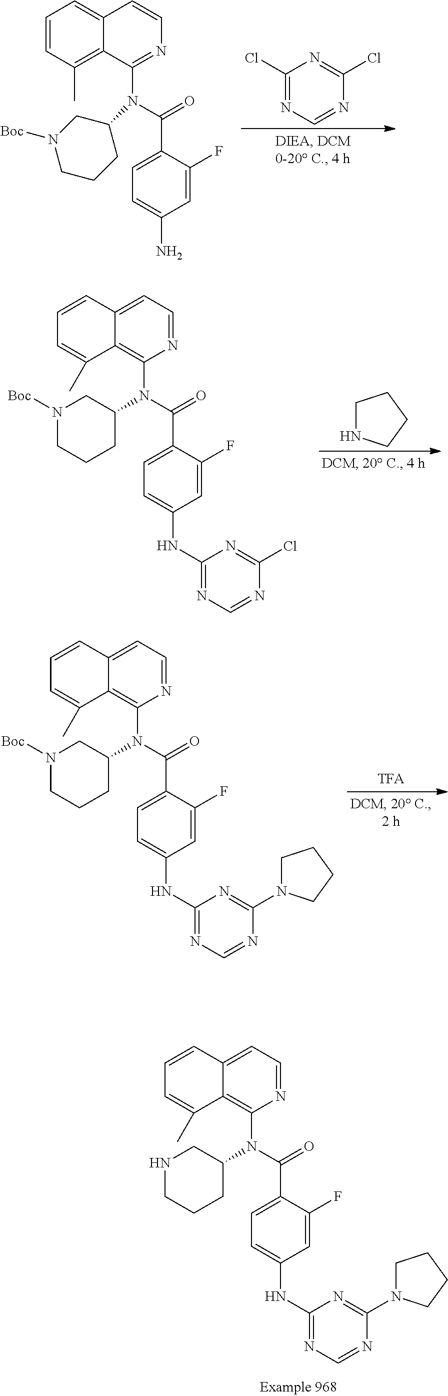 [SMILES: Cc1cccc2ccnc(N(C(=O)c3ccc(N)cc3F)[C@@H]3CCCN(C(=O)OC(C)(C)C)C3)c12 ; Clc1ncnc(Cl)n1 ; Cc1cccc2ccnc(N(C(=O)c3ccc(Nc4ncnc(Cl)n4)cc3F)[C@@H]3CCCN(C(=O)OC(C)(C)C)C3)c12 ; C1CCNC1 ; Cc1cccc2ccnc(N(C(=O)c3ccc(Nc4ncnc(N5CCCC5)n4)cc3F)[C@@H]3CCCN(C(=O)OC(C)(C)C)C3)c12 ; Cc1cccc2ccnc(N(C(=O)c3ccc(Nc4ncnc(N5CCCC5)n4)cc3F)[C@@H]3CCCNC3)c12]

### Step 1:

To a solution of tert-butyl (R)-3-(4-amino-2-fluoro-N-(8-methylisoquinolin-1-yl)benzamido)piperidine-1-carboxylate (3.00 g, 6.30 mmol) in DCM (50 mL) was added DIEA (2.43 g, 18.8 mmol) and 2,4-dichloro-1,3,5-triazine (1.00 g, 6.27 mmol) at 0° C. The mixture was stirred to room temperature for 4 h. Then the mixture was concentrated and the residue was purified by silica gel column chromatography, eluted with PE/EA (5/1) to afford tert-butyl (3R)-3-[N-(8-methylisoquinolin-1-yl)4-[(4-chloro-1,3,5-triazin-2-yl)amino]-2-fluorobenzamido]piperidine-1-carboxylate (2.51 g, 67% yield) as an off-white solid. LC-MS (ES, m/z): 592, 594 [M+H⁺].

### Step 2:

To a stirred solution of tert-butyl (3R)-3-[N-(8-methylisoquinolin-1-yl)4-[(4-chloro-1,3,5-triazin-2-yl)amino]-2-fluorobenzamido]piperidine-1-carboxylate (330 mg, 0.55 mmol) in DCM (5 mL) was added pyrrolidine (120 mg, 1.67 mmol). The resulting mixture was stirred at 20° C. for 4 h. The mixture was concentrated and the residue was purified by silica gel column chromatography using petroleum ether/EtOAc (3/1) to afford tert-butyl (3R)-3-[N-(8-methylisoquinolin-1-yl)2-fluoro-4-{[4-(pyrrolidin-1-yl)-1,3,5-triazin-2-yl]amino}benzamido]piperidine-1-carboxylate (220 mg, 63% yield) as a light-yellow solid. LC-MS (ES, m/z): 627 [M+H⁺].

### Step 3:

To a stirred solution of tert-butyl (3R)-3-[N-(8-methylisoquinolin-1-yl)2-fluoro-4-{[4-(pyrrolidin-1-yl)-1,3,5-triazin-2-yl]amino}benzamido]piperidine-1-carboxylate (220 mg, 0.35 mmol) in anhydrous DCM (5 mL) was added trifluoroacetic acid (1 mL) at room temperature and stirred for 2 h., The reaction mixture was concentrated under reduced pressure to give crude product which was further purified by Prep-HPLC (conditions: Column: XBridge Prep OBD C18 Column, 30*150 mm, 5 μm; Mobile Phase A: water (10 mmol/L NH₄HCO₃), Mobile Phase B: MeCN; Flow rate: 60 mL/min; Gradient: 5% B to 95% B in 8 min). The pure fraction was lyophilized to afford the title compound (60.2 mg, 33% yield) as a white solid. LC-MS (ES, m/z): 527 [M+H]⁺; ¹H NMR (400 MHZ, DMSO-d₆) δ (ppm): 10.12-10.56 (m, 1H), 8.51-8.37 (m, 1H), 8.31-7.93 (m, 1H), 7.80-7.64 (m, 2H), 7.59-7.41 (m, 3H), 6.99-6.88 (m, 1H), 6.70-6.55 (m, 1H), 4.71-4.42 (m, 1H), 3.64-3.25 (m, 8H), 3.07-2.82 (m, 2H), 2.80-2.70 (m, 3H), 2.00-1.82 (m, 4H), 1.69-1.55 (m, 2H), 1.31-1.01 (m, 1H).

### Synthesis of 2-fluoro-N-(8-methylisoquinolin-1-yl)-4-{[4-(piperidin-1-yl)-1,3,5-triazin-2-yl]amino}-N-[(3R)-piperidin-3-yl]benzamide (Example 969)

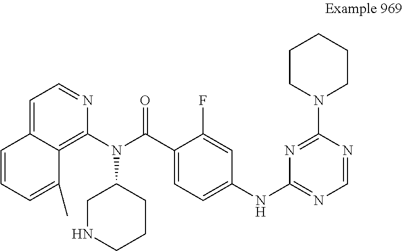 [SMILES: Cc1cccc2ccnc(N(C(=O)c3ccc(Nc4ncnc(N5CCCCC5)n4)cc3F)[C@@H]3CCCNC3)c12]

Example 969 was prepared according to the procedure described for Example 968 by substituting pyrrolidine with piperdine, provided the title compound as a white solid. LC-MS (ES, m/z): 541 [M+H]⁺; ¹H NMR (400 MHZ, DMSO-d₆) δ (ppm): 10.12-9.51 (m, 1H), 8.5-8.39 (m, 1H), 8.33-8.13 (m, 1H), 7.89-7.60 (m, 2H), 7.59-7.49 (m, 1H), 7.48-7.40 (m, 1H), 7.38-7.30 (m, 1H), 6.95-6.86 (m, 1H), 6.74-6.62 (m, 1H), 4.56-4.38 (m, 1H), 3.95-3.46 (m, 5H), 3.18-3.03 (m, 1H), 2.98-2.66 (m, 5H), 1.39-1.22 (m, 1H), 2.22-1.87 (m, 1H), 1.75-1.60 (m, 2H), 1.60-1.38 (m, 6H), 1.18-1.05 (m, 1H).

### Synthesis of 2-fluoro-N-(8-methylisoquinolin-1-yl)-4-{[4-(morpholin-4-yl)-1,3,5-triazin-2-yl]amino}-N-[(3R)-piperidin-3-yl]benzamide (Example 970)

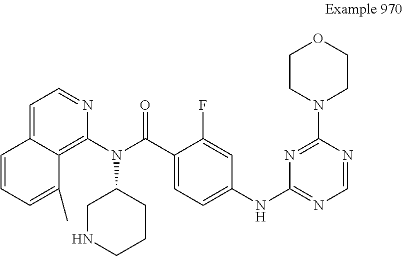 [SMILES: Cc1cccc2ccnc(N(C(=O)c3ccc(Nc4ncnc(N5CCOCC5)n4)cc3F)[C@@H]3CCCNC3)c12]

Example 970 was prepared according to the procedure described for Example 968 by substituting pyrrolidine with morpholine, provided the title compound as a white solid. LC-MS (ES, m/z): 543 [M+H]⁺; ¹H NMR (400 MHZ, DMSO-d₆) δ (ppm): 10.24-9.59 (m, 1H), 8.50-8.31 (m, 1H), 8.25-7.82 (m, 1H), 7.79-7.61 (m, 2H), 7.58-7.48 (m, 1H), 7.45-7.22 (m, 2H), 6.97-6.85 (m, 1H), 6.73-6.61 (m, 1H), 4.59-4.31 (m, 1H), 3.80-3.48 (m, 8H), 3.15-3.02 (m, 1H), 2.96-2.89 (m, 1H), 2.87-2.78 (m, 1H), 2.77-2.66 (m, 3H), 2.37-2.24 (m, 1H), 2.22-1.89 (m, 1H), 1.78-1.41 (m, 3H), 1.39-0.92 (m, 1H).

### Synthesis of (R)-2-fluoro-N-(8-methylisoquinolin-1-yl)-4-(4-phenyl-1,3,5-triazin-2-ylamino)-N-(piperidin-3-yl)benzamide (Example 971)

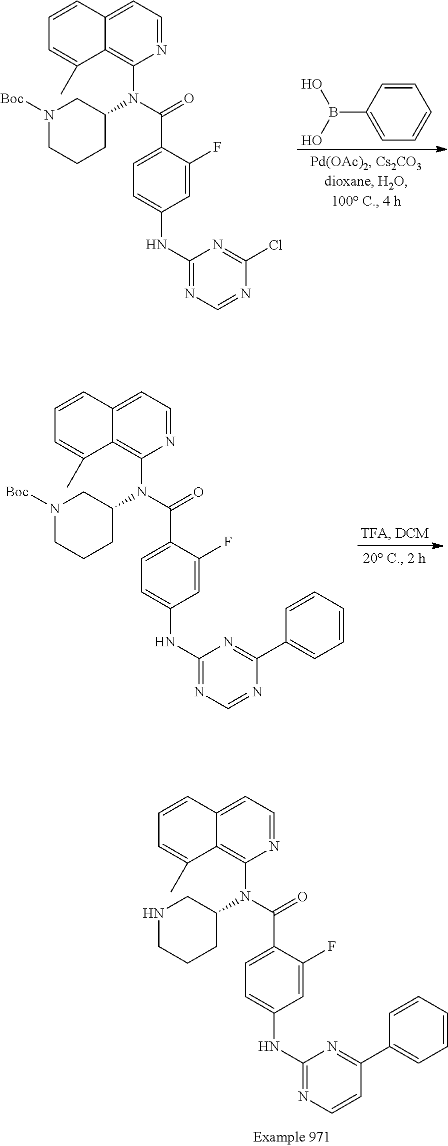 [SMILES: Cc1cccc2ccnc(N(C(=O)c3ccc(Nc4ncnc(Cl)n4)cc3F)[C@@H]3CCCN(C(=O)OC(C)(C)C)C3)c12 ; OB(O)c1ccccc1 ; Cc1cccc2ccnc(N(C(=O)c3ccc(Nc4ncnc(-c5ccccc5)n4)cc3F)[C@@H]3CCCN(C(=O)OC(C)(C)C)C3)c12 ; Cc1cccc2ccnc(N(C(=O)c3ccc(Nc4nccc(-c5ccccc5)n4)cc3F)[C@@H]3CCCNC3)c12]

### Step 1:

To a solution of tert-butyl (3R)-3-[N-(8-methylisoquinolin-1-yl)4-[(4-chloro-1,3,5-triazin-2-yl)amino]-2-fluorobenzamido]piperidine-1-carboxylate (500 mg, 0.844 mmol) and phenyl boronic acid (206 mg, 1.69 mmol) in 1,4-dioxane (10 mL) and H₂O (2 mL) were added Cs₂CO₃ (825 mg, 2.53 mmol) and Pd(OAc)₂ (20 mg, 0.084 mmol). After stirring for 4 h at 100° C. under a nitrogen atmosphere, the resulting mixture was allowed to cool down to room temperature. Then the mixture was filtered and the filtrate was concentrated. The residue was purified by silica gel column chromatography eluting with petroleum ether/EtOAc (1/1) to afford tert-butyl (3R)-3-[N-(8-methylisoquinolin-1-yl)2-fluoro-4-[(4-phenyl-1,3,5-triazin-2-yl)amino]benzamido]-piperidine-1-carboxylate (220 mg, 41% yield) as a light-brown solid.

### Step 2:

To a stirred solution of tert-butyl (3R)-3-[N-(8-methylisoquinolin-1-yl)2-fluoro-4-[(4-phenyl-1,3,5-triazin-2-yl)amino]benzamido]piperidine-1-carboxylate (220 mg, 0.35 mmol) in anhydrous DCM (5 mL) was added trifluoroacetic acid (1 mL) at 20° C. and stirred for 2 h. The reaction mixture was then concentrated to give crude product which was further purified by Prep-HPLC and the pure fraction was lyophilized to afford the title compound formic acid salt (60.9 mg, 30% yield) as a white solid. LC-MS (ES, m/z): 534 [M+H]⁺; ¹H NMR (400 MHz, DMSO-d₆) δ (ppm): 10.91-10.25 (m, 1H), 9.50-9.21 (m, 1H), 9.20-8.92 (m, 1H), 8.90-8.70 (m, 1H), 8.53-8.41 (m, 1H), 8.36-8.18 (m, 2H), 7.90-7.79 (m, 1H), 7.78-7.70 (m, 1H), 7.70-7.61 (m, 1H), 7.60-7.45 (m, 4H), 7.13-7.00 (m, 1H), 6.82-6.69 (m, 1H), 5.00-4.77 (m, 1H), 3.85-3.70 (m, 1H), 3.70-3.48 (m, 1H), 3.35-3.18 (m, 1H), 3.08-2.71 (m, 4H), 2.57-2.49 (m, 1H), 2.05-1.59 (m, 3H), 1.39-1.12 (m, 1H).

### Synthesis of (R)-4-((4-((4,4-difluoropiperidin-1-yl)methyl)pyrimidin-2-yl)amino)-N-(8-methylisoquinolin-1-yl)-N-(piperidin-3-yl)benzamide (Example 972)

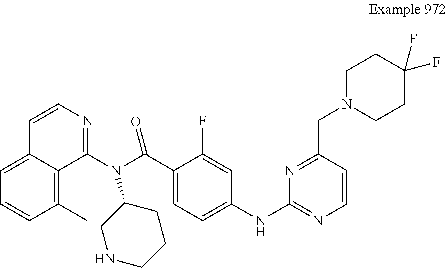 [SMILES: Cc1cccc2ccnc(N(C(=O)c3ccc(Nc4nccc(CN5CCC(F)(F)CC5)n4)cc3F)[C@@H]3CCCNC3)c12]

Example 972 was prepared according to the procedure described for Example 934 by substituting 1-methyl-piperazine with 4,4-difluoropiperidine, provided the title compound as an off-white solid. LC-MS (ES, m/z): 572 [M+H]⁺; ¹H-NMR (DMSO-d₆, 400 MHz) δ (ppm): 9.72-9.08 (m, 2H), 8.54-8.49 (m, 1H), 8.48-8.42 (m, 1H), 7.91-7.87 (m, 1H), 7.79-7.77 (m, 1H), 7.56-7.50 (m, 1H), 7.43-7.38 (m, 2H), 7.02-6.90 (m, 2H), 5.00-4.94 (m, 1H), 4.80-4.12 (m, 1H), 3.71-3.64 (m, 1H), 3.53-3.34 (m, 2H), 3.31-3.01 (m, 2H), 2.98-2.77 (m, 2H), 2.77-2.62 (m, 4H), 2.47-2.33 (m, 2H), 2.18-1.90 (m, 2H), 1.89-1.78 (m, 1H), 1.77-1.60 (m, 1H), 1.21-1.14 (m, 1H).

### BIOLOGICAL EXAMPLES

### In Vitro Translation Materials and Methods

Cloning. Target ORFs were synthesized with a C-terminal nanoluciferase reporter. As necessary, an N-terminal 1×FLAG tag was appended for improved expression. Synthesized fusions were cloned into the pT7CFE1 vector (Thermo Fisher) at the BtgI and BglII sites.

Messenger RNA synthesis. DNA templates were linearized with Pmel enzyme (NEB) purified with Monarch DNA cleanup kit (NEB). Messenger RNA (mRNA) was transcribed in vitro using a T7 HighScribe kit (NEB) containing 0.1 μg/μL DNA template. Synthesized mRNA was purified on Monarch RNA cleanup columns (NEB).

In vitro translation dose-response curves. Dose response curve IVTs were performed similar to the single point IVT screening with the following changes. The desired concentration curves were plated into 384 well plates from DMSO stocks using a Tecan D300e dispenser. As before, 0.5% DMSO and puromycin were used as negative and positive controls, respectively. After reaction incubation and luminescence measurement, dose-response curves were fit to the data using Prism (Graphpad) or Collaborative Drug Discovery analysis programs

HeLa S3 cell Preparation. HeLa S3 cells were grown essentially as described in Mikami 2010. Frozen aliquots of banked HeLa cells (ATCC #CCL-2.2) were thawed and diluted into agitated suspension culture in CD293 medium supplemented with 10% HI FBS, 1× GlutaMAX, and 1× Pen/Strep. Cells were expanded in suspension over 10 passages to inoculate 3×20 L wave bag bioreactors and cultured for approximately 48 h to a final density of 1.81×106 viable cells per mL. Cells were pelleted by centrifugation at 2500 RCF for 15 min at 4° C. Two 1 L pellets were combined in 32 sets and re-pelleted in 50 ml conical tubes, resulting in 32×10 mL cell pellets weighing approximately 10 g each. Final pellets were snap frozen in liquid nitrogen and stored at −80° C. (cell culture, harvesting, and cryo-preservation completed at LakePharma). Cell pellets were subsequently thawed at RT, resuspended in 15 mL wash buffer (35 mM HEPES-KOH, pH 7.5, 140 mM NaCl, and 11 mM glucose), then re-pelleted by centrifugation at 2500 RCF for 10 min at 4° C. The supernatant was discarded and the wash step repeated 2×. The pellets were resuspended each pellet in 10 mL in hypertonic extraction buffer (20 mM HEPES-KOH, pH 7.5, 45 mM potassium acetate, 45 mM KCl, 1.8 mM magnesium acetate, and 1 mM TCEP added directly before use). The cell suspension was transferred to a cell disruption vessel (Parr Instrument Company, model 4636, 1850 mL capacity) and incubated for 15 min on ice, then sealed under nitrogen (200 PSI) for 1.5 h. The cell homogenates were released through a needle valve and collected on ice, then diluted with 1/29 volume high potassium buffer (20 mM HEPES-KOH, pH 7.5, 945 mM potassium acetate, 945 mM KCl, 1.8 mM magnesium acetate, and 1 mM TCEP). The cell homogenates were then clarified by centrifugation at 2500 RCF for 10 min at 4° C. and the supernatant collected. The clarification step was repeated 3×. The resulting cell extracts (675 mL, 2.5 mg/mL total protein) were aliquoted and flash frozen in liquid nitrogen and stored at −80° C. until use.

### Example 1. In Vitro Single Point Translation Screening Assay

Compound screening was performed using 12 μL or 20 μl in vitro translation (IVT) reactions in 384 well plates at compound concentrations of 50 M or 41.7 UM and 0.5% final concentration DMSO. A single reaction consisted of 50% v/v HeLa lysate, 10% v/v reaction mix 1 (270 mM HEPES-KOH pH 7.5, 520 mM KOAc, 16 mM MgOAc₂, 45 mM TCEP), 10% v/v reaction mix 2 (12.6 mM ATP, 1.2 mM GTP, 200 mM creatine phosphate, 0.6 mg/mL creatine kinase (Millipore), 1.7×MEM essential amino acids (Gibco), 3.4×MEM nonessential amino acids (Gibco), 0.7 mM glutamine, 4 U/μL RNasin Recombinant RNase inhibitor (Promega)), and 5 ng/μL target mRNA. Translation reactions were incubated for 20 min at 30° C. Nanoluciferase luminescence was measured by combining 2 μL of IVT reaction with 20 μL of prepared reagent from the Nanoglo assay kit (Promega). A negative control of 0.5% DMSO and positive control of 50 μM puromycin were used to define to assay window. Representative data is given in Table 5.

TABLE 5
Percent Inhibition of c-MYC as Measured in High Throughput
In Vitro Single Point Translation Screening Assay

| | Average % | std | Average % | std | Average % | |
|---|---|---|---|---|---|---|
| | inhibition | deviation | inhibition | deviation | inhibition | std deviation |
| Example # | 20 μL: Thermo Lysates | | 12 μL: Thermo Lysates | | 12 μL: Initial Lysates | |
| 1 | not tested | not tested | not tested | not tested | not tested | not tested |
| 2 | not tested | not tested | not tested | not tested | not tested | not tested |
| 3 | not tested | not tested | not tested | not tested | not tested | not tested |
| 4 | 41.7 | 12.3 | | | | |
| 5 | not tested | not tested | not tested | not tested | not tested | not tested |
| 6 | not tested | not tested | not tested | not tested | not tested | not tested |
| 7 | 40.7 | 13.3 | | | | |
| 8 | 34.9 | 12.7 | | | | |
| 9 | 37.8 | 20.4 | | | | |
| 10 | 30.9 | 3.59 | | | | |
| 11 | 37.2 | 6.55 | | | | |
| 12 | 40 | 13.2 | | | | |
| 13 | 39.2 | 11.4 | | | | |
| 14 | 31.2 | 8.61 | | | | |
| 15 | 42.2 | 15.4 | | | | |
| 16 | 33.1 | 17.3 | | | | |
| 17 | 30 | 20.7 | | | | |
| 18 | 30.3 | 13.2 | | | | |
| 19 | 30.1 | 9.43 | | | | |
| 20 | 36.7 | 11.1 | | | | |
| 21 | 33 | 10.2 | | | | |
| 22 | 51.2 | 14.1 | | | | |
| 23 | 60.9 | 2.09 | | | | |
| 24 | 47.7 | 12.9 | | | | |
| 25 | 37.5 | 12.5 | | | | |
| 26 | 35.7 | 7.24 | | | | |
| 27 | 33.8 | 2.78 | | | | |
| 28 | 46.1 | 6.17 | | | | |
| 29 | 50.7 | 12.6 | | | | |
| 30 | 43.2 | 22.3 | | | | |
| 31 | 48.3 | 23.8 | | | | |
| 32 | | | 92.2 | 1.27 | | |
| 33 | 30.2 | 10.3 | | | | |
| 34 | 38 | 16.2 | | | | |
| 35 | 40.3 | 7.45 | | | | |
| 36 | | | | | 36.5 | 18.3 |
| 37 | 62.9 | 5.31 | | | | |
| 38 | 33.5 | 14.4 | | | | |
| 39 | 75.3 | 13.7 | | | | |
| 40 | 43.3 | 23.1 | | | | |
| 41 | 42.7 | 13.6 | | | | |
| 42 | 59 | 11.4 | | | | |
| 43 | 49.5 | 9.39 | | | | |
| 44 | 38.1 | 13.4 | | | | |
| 45 | 33.5 | 18 | | | | |
| 46 | | | 30 | 32.7 | | |
| 47 | | | 25.9 | 9.63 | | |
| 48 | | | 42.7 | 9.78 | | |
| 49 | | | 38.5 | 5.59 | | |
| 50 | | | 30 | 9.4 | | |
| 51 | | | 12.6 | 12.4 | | |
| 52 | | | 24.3 | 8.35 | | |
| 53 | | | 34.5 | 2.95 | | |
| 54 | | | 15.2 | 10.1 | | |
| 55 | | | 20.5 | 4.58 | | |
| 56 | | | 15.7 | 5.49 | | |
| 57 | | | 18.5 | 4.63 | | |
| 58 | | | 30.4 | 24.9 | | |
| 59 | | | 17.5 | 5.16 | | |
| 60 | | | 16.1 | 4.15 | | |
| 61 | | | 30.6 | 7.97 | | |
| 62 | | | 21.2 | 4.17 | | |
| 63 | | | 23.5 | 16.6 | | |
| 64 | | | | | 30.3 | 9.77 |
| 65 | | | | | 31.3 | 31 |
| 66 | | | | | 33.9 | 15.7 |
| 67 | | | | | 32.1 | 25.7 |
| 68 | | | | | 52 | 19.2 |
| 69 | | | | | 93.1 | 4.03 |
| 70 | | | | | 65.9 | 1.7 |
| 71 | | | | | 38.2 | 7.28 |
| 72 | | | | | 55.1 | 3.79 |
| 73 | | | | | 78 | 1.82 |
| 74 | | | | | 35.9 | 16.7 |
| 75 | | | | | 31.5 | 14.9 |
| 76 | | | | | 36.8 | 24.3 |
| 77 | | | | | 30.3 | 8.52 |
| 78 | | | | | 31.4 | 17 |
| 79 | | | | | 32.4 | 19.5 |
| 80 | | | | | 32.5 | 22.9 |
| 81 | | | | | 32.6 | 14.2 |
| 82 | | | | | 33 | 12.1 |
| 83 | | | | | 33.5 | 5.01 |
| 84 | | | | | 29.5 | 24.2 |
| 85 | | | | | 34.8 | 18.6 |
| 86 | | | | | 78 | 14 |
| 87 | | | | | 33 | 26 |
| 88 | | | | | 30.3 | 2.74 |
| 89 | | | | | 36.6 | 9.2 |
| 90 | | | | | 37.5 | 21.7 |
| 91 | | | | | 30.4 | 9.44 |
| 92 | | | | | 31.1 | 22.4 |
| 93 | | | | | 38.4 | 11.5 |
| 94 | | | | | 33.2 | 12.8 |
| 95 | | | | | 37.7 | 9.8 |
| 96 | | | | | 38.5 | 9.42 |

### Example 2. In Vitro Translation Dose-Response Curves

Dose response curve IVTs were performed similar to the single point IVT screening with the following changes. The desired concentration curves were plated into 384 well plates from DMSO stocks using a Tecan D300e dispenser. As before, 0.5% DMSO and puromycin were used as negative and positive controls, respectively. After reaction incubation and luminescence measurement, dose-response curves were fit to the data using Prism (Graphpad) or Collaborative Drug Discovery analysis programs.

TABLE 6
IVT dose response curve data. IC50s of the indicated
compounds were measured as described in a In Vitro
Multiple Point Translation Screening Assay.

| Example # | Geomean pIC50 | S.D. |
|---|---|---|
| 50 | 6.0 (n = 4) | 0.3 |
| 54 | 4.70 (n = 2) | 0.05 |
| 57 | 4.6 (n = 2) | 0.2 |
| 51 | 4.8 (n = 2) | 0.3 |

Each of the compounds in Table 5 were tested in the In Vitro Multiple Point Translation Screening Assay and in every case the pIC50 was >4.3 (IC₅₀<50 micromolar).

### Example 3. Cellular c-MYC Alpha Screen Data

HCT-116 Firefly-luciferase reporter cells (BPS bioscience) were seeded in 96-well tissue-culture treated sterile plates (Greiner Bio-One) at 7.5e4 cells/mL in 100 μL complete growth media (McCoy's 5A+10% FBS, +1% Pen-Strep, +1% G418). Cells were incubated at 37° C. with 5% CO2 for 24 h then treated with 3× compound solution in 50 μL complete growth media for a final volume of 150 μL per well with 0.2% DMSO. After 24 h incubation at 37° C. with 5% CO2, plates were allowed to equilibrate to RT for 20 min; the media was then aspirated, and the plate washed once with 200 μL 1×PBS. Cells were then lysed by addition of 1× Alpha Screen lysis buffer (50 μL) from the Perkin Elmer alpha Screen c-MYC detection kit (Part number 6760611C). The lysate is then transferred to ice and advanced to the Alpha Screen detection assay or sealed with adhesive foil and stored at −80° C. for up to 2 weeks before use.

The alpha screen detection assay was then performed as per the vendors instructions. Briefly the acceptor bead mix was prepared as per vendors instructions. 30 ul of the cell lysate is transferred to a 96-well white assay plate. 15 ul of the acceptor mix was added to each well and incubated in the dark for at least one hour. Then the donor bead mix was prepared as per vendors instructions and 15 ul of this was added to the assay plate. The plate was then incubated at RT for one hour in the dark. The plate was then read in the SpectraMax i3x plate reader (Molecular Devices) using the alpha screen module (excitation wavelength 680 emission wavelength 615) and analyzed using GraphPad Prism 9 software.

TABLE 7
c-MYC alpha screen dose response curve data.
IC50s of the indicated compounds
were measured as described.

| Example # | Geomean pIC50 |
|---|---|
| 50 | 6.8 (n = 1) |
| 51 | 6.0 (n = 1) |
| 243 | 6.4 (n = 1) |

Compounds 50 and 51 were tested in the c-MYC alpha screen assay and the pIC50s are as indicated.

### Example 4. Cellular c-MYC Reporter Assay

HCT-116 Firefly-luciferase reporter cells (BPS Bioscience) were seeded in 96-well tissue-culture treated sterile plates (Greiner Bio-One) at 5e4 cells/mL in 100 μL complete growth media (McCoy's 5A+10% FBS, +1% Pen-Strep, +1% G418). Cells were incubated at 37° C. with 5% CO2 for 4-6 h then treated with 3× compound solution in 50 μL complete growth media for a final volume of 150 μL per well with 0.2% DMSO. After 24 h incubation at 37° C. with 5% CO2, plates were allowed to equilibrate to RT for 20 min then developed by addition of 100 μL ONE-Glo Luciferase detection reagent (Promega), incubated at RT for 10 min. Luminescence was recorded using a SpectraMax i3x plate reader (Molecular Devices) and analyzed using Prism GraphPad 9 software.

TABLE 8a
Cellular c-MYC reporter assay dose response
curve data. EC50s of the indicated
compounds were measured as described.

| Example # | Geomean pEC50 | S.D. |
|---|---|---|
| 50 | 7.6 (n = 2) | 0.8 |
| 57 | 6.59 (n = 2) | 0.08 |
| 51 | 6.6 (n = 5) | 0.7 |
| 50 | 6.2 (n = 2) | 0.3 |

| Example # | pIC50 |
|---|---|
| 97 | 6.42 |
| 98 | 6.12 |
| 99 | 5.7 |
| 100 | 5.93 |
| 101 | <5.00 |
| 102 | 7.33 |
| 103 | 6.11 |
| 104 | 5.05 |
| 105 | 5.2 |
| 106 | 6.27 |
| 107 | 6.01 |
| 108 | 6.89 |
| 109 | <5.00 |
| 110 | <5.00 |
| 111 | 6.29 |
| 112 | 6.42 |
| 113 | 5.37 |
| 114 | 5.95 |
| 115 | 6.26 |
| 116 | 5.59 |
| 117 | 6.31 |
| 118 | <4.70 |
| 119 | 5.85 |
| 120 | 6.15 |
| 121 | 7.42 |
| 122 | 6.91 |
| 123 | 5.3 |
| 124 | 5.41 |
| 125 | 7.61 |
| 126 | 6.45 |
| 127 | 6.47 |
| 128 | <5.00 |
| 129 | 6.47 |
| 130 | 5.64 |
| 131 | 5.72 |
| 132 | 5.8 |
| 133 | 6.57 |
| 134 | 6.24 |
| 135 | 6.24 |
| 136 | 5.96 |
| 137 | 5.53 |
| 138 | 7.35 |
| 139 | 6.77 |
| 140 | 6.62 |
| 141 | 5.72 |
| 142 | 7.1 |
| 143 | 6.14 |
| 144 | 6.22 |
| 145 | 6.28 |
| 146 | 6.25 |
| 147 | 5.86 |
| 148 | 4.86 |
| 149 | 5.42 |
| 150 | 5.64 |
| 151 | 5.99 |
| 152 | 6.33 |
| 153 | 5.62 |
| 154 | 5.98 |
| 155 | 5.24 |
| 156 | 5.15 |
| 157 | 6.3 |
| 158 | 6.16 |
| 159 | 5.58 |
| 160 | 6.35 |
| 161 | 6.38 |
| 162 | 6.11 |
| 163 | 6.27 |
| 164 | 7.01 |
| 165 | 6.77 |
| 166 | 6.85 |
| 167 | 6.18 |
| 168 | 5.28 |
| 169 | 6.5 |
| 170 | 7.25 |
| 171 | 6.54 |
| 172 | 5.22 |
| 173 | 6.6 |
| 174 | 5.45 |
| 175 | 5.03 |
| 176 | 6.03 |
| 177 | 6.45 |
| 178 | 5.91 |
| 179 | 7.14 |
| 180 | 5.47 |
| 181 | 4.96 |
| 182 | 6.05 |
| 183 | 6.72 |
| 184 | 5.81 |
| 185 | 5.48 |
| 186 | 6.7 |
| 187 | 7.3 |
| 188 | 5.23 |
| 189 | 5.25 |
| 190 | 5.23 |
| 191 | 7.97 |
| 192 | 6.77 |
| 193 | 7.1 |
| 194 | 5.92 |
| 195 | 7.06 |
| 196 | <4.7 |
| 197 | 6.16 |
| 198 | 6.21 |
| 199 | 6.58 |
| 200 | 7.11 |
| 201 | 6.83 |
| 202 | 6.43 |
| 203 | 6.89 |
| 204 | 6.32 |
| 205 | 6.77 |
| 206 | 4.81 |
| 207 | 7.38 |
| 208 | 5.45 |
| 209 | 6.85 |
| 210 | 6.56 |
| 211 | not reported |
| 212 | 6.45 |
| 213 | 6.19 |
| 214 | not reported |
| 215 | 6.57 |
| 216 | 6.36 |
| 217 | 7.27 |
| 218 | 7.36 |
| 219 | 5.2 |
| 220 | not reported |
| 221 | 5.7 |
| 222 | 7.03 |
| 223 | 6.67 |
| 224 | <4.70 |
| 225 | 5.32 |
| 226 | <4.70 |
| 227 | <4.70 |
| 228 | <4.70 |
| 229 | 4.82 |
| 230 | not reported |
| 231 | <4.70 |
| 232 | 5.07 |
| 233 | 6.15 |
| 234 | 6.4 |
| 235 | 5.07 |
| 236 | 6.78 |
| 237 | 6.23 |
| 238 | 6.62 |
| 239 | 6.36 |
| 240 | not reported |
| 241 | 7.22 |
| 242 | 7.42 |
| 243 | 6.28 |
| 244 | 6.97 |
| 245 | 6.81 |
| 246 | 7.48 |
| 247 | 5.3 |
| 248 | 6.07 |
| 249 | 5.85 |
| 250 | 6.09 |
| 251 | 7.17 |
| 252 | 5.85 |
| 253 | 6.94 |
| 254 | 7.21 |
| 255 | 7 |
| 256 | 5.04 |
| 257 | 5.38 |
| 258 | 5.14 |
| 259 | 5.08 |
| 260 | 5.28 |
| 261 | 5.26 |
| 262 | 6.24 |
| 263 | 5.9 |
| 264 | 5.4 |
| 265 | 5.24 |
| 266 | 5.53 |
| 267 | 5.27 |
| 268 | 5.23 |
| 269 | 5.45 |
| 270 | 5.7 |
| 271 | 5.32 |
| 272 | 6.36 |
| 273 | 5.77 |
| 274 | 5.42 |
| 275 | 5.41 |
| 276 | 5.15 |
| 277 | 5.31 |
| 278 | 6.31 |
| 279 | 5.29 |
| 280 | 6.05 |
| 281 | 5.58 |
| 282 | 5.38 |
| 283 | 5.92 |
| 284 | 5.92 |
| 285 | 6.53 |
| 286 | 6.71 |
| 287 | 5.23 |
| 288 | 5.87 |
| 289 | <5.23 |
| 290 | 5.47 |
| 291 | 5.90 |
| 292 | <4.70 |
| 293 | <4.70 |
| 294 | <5.00 |
| 295 | <4.70 |
| 296 | <4.70 |
| 297 | 5.11 |
| 298 | 6.58 |
| 299 | 5.26 |
| 300 | <4.7 |
| 301 | <5.00 |

Additional compounds from Table 5 were tested in this assay and in the majority of cases the cellular pEC50s were >6 (EC50<1 micromolar).

### Example 5. Assessment of Efficacy of Compounds in HCT-116 Mouse Xenograft Model

HCT-116 cells (ATCC CCL-247) harvested from tissue culture flasks are implanted into the subcutaneous space of the left flank of the mice. Resulting tumors are monitored by calipering twice weekly. Treatment starts after randomization when tumor volumes have reached a size of approx. 200 mm³. Dosing with test compound or vehicle is b.i.d. (2 times/day) by oral gavage four days a week for four weeks. Average tumor volume is measured over time and if a significant reduction in tumor size is observed in response to compound dosing, this data provides evidence of in vivo activity of the compound.

### Example 6. Assessment of Efficacy of Compounds in P493-6 Mouse Xenograft Model

P493-6 is a human-derived B-cell Burkitt's lymphoma line that has been studied extensively as a c-MYC dependent human B-cell tumor that forms tumors in mouse SCID xenotransplantation models (Schuhmacher 1999). The cells are inoculated s.c. into the flank of 7-week old SCID mice. Treatment starts after randomization when tumor volumes have reached a size of approx. 200 mm³. Dosing with test compound or vehicle is 2 times a day by oral gavage four days a week for four weeks. Average tumor volume is measured over time and if a significant reduction in tumor size is observed in response to compound dosing, this data provides evidence of in vivo activity of the compound.

### Example 7. Assessment of Efficacy of Compounds in 4188 Human c-MYC Transgenic Mouse Model (Expresses High Levels of Human c-MYC High MYC)

Immortalized T-ALL (T-cell acute lymphoblastic leukemia) cells derived from a well characterized human c-MYC transgenic mouse model, to yield the 4188 cell line (Felsher 1999). The 4188 cells are injected intraperitoneally into syngeneic mice to create tumors in the thymus, spleen, liver and GI tract. Test compound is dosed t.i.d. at doses of 10, 30, 100 and 300 mg/kg for four weeks. Mice are observed for up to 60 weeks and scored for survival. Exemplary literature data is given below. Mortality is measured over time and if a significant reduction in mortality is observed in response to compound dosing, this data provides evidence of in vivo activity of the compound.

### Example 8. Assessment of Efficacy of Compounds in Liver-Specific Transgenic Mouse Model LAP-tTa/tet-O-MYC

This is a conditional transgenic mouse model of c-MYC-driven hepatocellular carcinoma. Conditional overexpression of human c-MYC in the liver induces hepatocellular cancer whereas c-MYC inactivation by tetracycline results in sustained tumor regression (Shachaf CM, 2004). Test compound is dosed b.i.d. at doses of 10, 30, 100 and 300 mg/kg for four weeks. Mice are observed for up to 50 weeks and scored for survival. Mortality is measured over time and if a significant reduction in mortality is observed in response to compound dosing, this data provides evidence of in vivo activity of the compound.

### Example 10. Assessment of Efficacy of Compounds in Kidney-Specific Transgenic Mouse Model GGT-tTa/tet-O-MYC

This is a conditional transgenic mouse model of c-MYC-driven renal cell carcinoma. Conditional c-MYC overexpression in the kidney induces renal cell cancer whereas c-MYC inactivation by tetracycline results in sustained tumor regression (Shroff 2015). In a positive control arm, mice receive 0.1 mg/mL of doxycycline via drinking water until 4 weeks of age and then are taken off doxycycline. The mice are imaged by MRI to screen for tumors and then split into cohorts for vehicle control and test compound treatment. In the test arms, the test compound is dosed b.i.d. at doses of 10, 30, 100 and 300 mg/kg for four weeks. Mice are observed for up to 50 weeks and scored for survival and tumor volume. Average tumor volume is measured over time and if a significant reduction in tumor size is observed in response to compound dosing, this data provides evidence of in vivo activity of the compound.

### Example 11. Assessment of Efficacy of Compounds in Hepatocellular Carcinoma with HepG2 Cells

HepG2 cells (5×10⁶ cells/mouse) are injected subcutaneously in the flank of non-severe combined immunodeficiency (SCID)-gamma (NSG) mice, which are 4-6 weeks old (Dhanasckaran 2020). Mice are observed daily until a small tumor is palpable. Mice are then randomized to receive vehicle or test compound. Test compound is dosed b.i.d. at doses of 10, 30, 100 and 300 mg/kg for four weeks. Mice are observed for up to 65 days and scored for survival. Average tumor volume is measured over time and if a significant reduction in tumor size is observed in response to compound dosing, this data provides evidence of in vivo activity of the compound.

### Example 12. Assessment of Efficacy of Compounds with Cell Viability Assay in MV-4-11 Cells

MV-4-11 cells (ATCC) were seeded into sterile 96-well flat clear bottom white polystyrene TC-treated microplates (Corning) at 7,000 cells/well in 150 μL complete growth medium (Iscove's Modified Dulbecco's Medium (IMDM) (Gibco), 10% Heat Inactivated Fetal Bovine Serum (HyClone), 1% Penicillin-Streptomycin-L-Glutamine (HyClone), 1% GlutaMAX (Gibco)). Within 1 h of seeding, cells were treated with compounds (9-pt. serial dilution (1:3) in DMSO up to 10 μM) using a Tecan D300e reagent dispenser (Tecan) to a final volume of 150 μL media with 0.2% DMSO, then returned to incubator at 37° C., 5% CO₂. After 72 h incubation, plates were removed from incubators and allowed to equilibrate to room temperature (˜10 min) before adding 50 μL per well Cell-Titer Glo reagent (Promega) using a WellJet (Integra Biosciences) dispenser. Plates were then incubated at room temperature for 2-20 min prior to measurement of luminescence on an Envision (Perkin Elmer) plate reader. Data were normalized to DMSO (100% viability) and analyzed using a non-linear four-parameter curve fit.

### Table 9:

| Example # | IC50 (nM) |
|---|---|
| 302 | 480 |
| 303 | 450 |
| 304 | 37 |
| 305 | 1600 |
| 306 | 58 |
| 307 | 100 |
| 308 | 25 |
| 309 | 280 |
| 310 | 28 |
| 311 | 23 |
| 312 | 2600 |
| 313 | 230 |
| 314 | 50 |
| 315 | 240 |
| 316 | 82 |
| 317 | 79 |
| 318 | 220 |
| 319 | 66 |
| 320 | 5.6 |
| 321 | 780 |
| 322 | 180 |
| 323 | 440 |
| 324 | 1500 |
| 325 | 1500 |
| 326 | 67 |
| 327 | 2700 |
| 328 | 210 |
| 329 | 72 |
| 330 | 90 |
| 331 | 81 |
| 332 | 2700 |
| 333 | >10E+03 |
| 334 | >10E+03 |
| 335 | 2600 |
| 336 | 270 |
| 337 | 250 |
| 338 | >10E+03 |
| 339 | 550 |
| 340 | 860 |
| 341 | 370 |
| 342 | 2000 |
| 343 | 1400 |
| 344 | 930 |
| 345 | 300 |
| 346 | >10E+03 |
| 347 | 300 |
| 348 | 460 |
| 349 | 310 |
| 353 | 540 |
| 354 | 970 |
| 361 | >10E+03 |
| 362 | 4300 |
| 363 | 350 |
| 364 | 360 |
| 365 | 480 |
| 366 | 290 |
| 367 | 250 |
| 368 | 500 |
| 369 | 230 |
| 370 | 180 |
| 371 | 1300 |
| 372 | 100 |
| 373 | 180 |
| 374 | 600 |
| 375 | 600 |
| 376 | 680 |
| 377 | 1300 |
| 378 | 190 |
| 379 | >10E+03 |
| 380 | 120 |
| 381 | 200 |
| 382 | 640 |
| 383 | 200 |
| 384 | 110 |
| 385 | 520 |
| 386 | 330 |
| 387 | 300 |
| 388 | 76 |
| 389 | >10E+03 |
| 390 | 240 |
| 391 | >10E+03 |
| 392 | 840 |
| 393 | 150 |
| 394 | 140 |
| 395 | 230 |
| 396 | 150 |
| 397 | 460 |
| 398 | 71 |
| 399 | 1100 |
| 400 | 2400 |
| 401 | >10E+03 |
| 402 | 290 |
| 403 | 2100 |
| 405 | 7800 |
| 406 | >10E+03 |
| 407 | >10E+03 |
| 408 | 5.3 |
| 409 | 54 |
| 410 | 190 |
| 411 | 390 |
| 412 | 63 |
| 413 | 160 |
| 414 | 220 |
| 415 | 1200 |
| 416 | 180 |
| 417 | 390 |
| 418 | 360 |
| 419 | 200 |
| 420 | 330 |
| 421 | 16 |
| 422 | 6.6 |
| 423 | 260 |
| 424 | 110 |
| 425 | 230 |
| 426 | 160 |
| 427 | 210 |
| 428 | 250 |
| 429 | 1000 |
| 430 | 650 |
| 431 | 2300 |
| 432 | 400 |
| 433 | 26 |
| 434 | 91 |
| 435 | 300 |
| 436 | 270 |
| 437 | 380 |
| 438 | 220 |
| 439 | 1200 |
| 440 | 290 |
| 441 | 120 |
| 442 | 1000 |
| 443 | 300 |
| 444 | 6.9 |
| 445 | 290 |
| 446 | 1900 |
| 447 | 110 |
| 448 | 52 |
| 449 | 940 |
| 450 | 300 |
| 451 | 250 |
| 452 | 180 |
| 453 | 200 |
| 454 | 1000 |
| 455 | 650 |
| 456 | 380 |
| 457 | 290 |
| 458 | 390 |
| 459 | 430 |
| 460 | 220 |
| 461 | 720 |
| 462 | 1000 |
| 463 | 320 |
| 464 | 640 |
| 465 | 240 |
| 466 | 440 |
| 467 | 220 |
| 468 | 120 |
| 469 | 180 |
| 470 | 91 |
| 471 | 240 |
| 472 | 580 |
| 473 | 300 |
| 474 | 9.9 |
| 475 | 4600 |
| 476 | 220 |
| 477 | 2800 |
| 478 | 340 |
| 479 | 3200 |
| 480 | 540 |
| 481 | 270 |
| 482 | 870 |
| 483 | 340 |
| 484 | 930 |
| 485 | 980 |
| 486 | >10E+03 |
| 487 | 620 |
| 488 | 650 |
| 489 | >10E+03 |
| 490 | 300 |
| 491 | 460 |
| 492 | 870 |
| 493 | 530 |
| 494 | >10E+03 |
| 495 | 660 |
| 496 | 5400 |
| 497 | 2000 |
| 498 | 2000 |
| 499 | 1700 |
| 500 | >10E+03 |
| 501 | 800 |
| 502 | 2100 |
| 503 | 2300 |
| 504 | 810 |
| 505 | 250 |
| 506 | 710 |
| 507 | 610 |
| 508 | 300 |
| 509 | 520 |
| 510 | 260 |
| 511 | 250 |
| 512 | 1100 |
| 513 | 540 |
| 514 | 890 |
| 515 | 2100 |
| 516 | 750 |
| 517 | 7300 |
| 518 | 940 |
| 519 | 430 |
| 520 | 1700 |
| 521 | 760 |
| 522 | 770 |
| 523 | 920 |
| 524 | >10E+03 |
| 525 | 790 |
| 526 | 870 |
| 527 | 200 |
| 528 | 870 |
| 529 | 140 |
| 530 | 1600 |
| 531 | 430 |
| 532 | 1800 |
| 533 | 910 |
| 534 | 200 |
| 535 | 1200 |
| 536 | >10E+03 |
| 537 | 1600 |
| 538 | 920 |
| 539 | 630 |
| 540 | 470 |
| 541 | 640 |
| 542 | 6300 |
| 543 | 310 |
| 544 | 1800 |
| 545 | 5300 |
| 546 | 92 |
| 547 | 480 |
| 548 | 240 |
| 549 | 1500 |
| 550 | >6.0E+03 |
| 551 | 520 |
| 552 | 910 |
| 553 | 78 |
| 556 | 650 |
| 557 | 130 |
| 558 | 11 |
| 559 | 34 |
| 560 | 220 |
| 561 | 1200 |
| 562 | 340 |
| 563 | 340 |
| 564 | 14 |
| 565 | 230 |
| 566 | 240 |
| 567 | 230 |
| 568 | 89 |
| 569 | 570 |
| 570 | 110 |
| 571 | 63 |
| 572 | 89 |
| 573 | 230 |
| 574 | 390 |
| 575 | 150 |
| 576 | 430 |
| 577 | 76 |
| 578 | 45 |
| 579 | 220 |
| 580 | 430 |
| 581 | 340 |
| 582 | 220 |
| 583 | 4.2 |
| 584 | 300 |
| 585 | 440 |
| 586 | 310 |
| 587 | 14 |
| 588 | 790 |
| 589 | 1000 |
| 590 | >10E+03 |
| 591 | >2.0E+03 |
| 592 | 360 |
| 593 | 780 |
| 594 | 190 |
| 595 | 470 |
| 596 | 890 |
| 597 | 190 |
| 598 | 320 |
| 599 | 240 |
| 600 | 270 |
| 601 | 200 |
| 602 | 460 |
| 603 | 240 |
| 604 | 2100 |
| 605 | 470 |
| 606 | 400 |
| 607 | 800 |
| 608 | 4300 |
| 609 | 400 |
| 610 | 86 |
| 611 | 2300 |
| 612 | 280 |
| 613 | 460 |
| 614 | 16 |
| 615 | 17 |
| 617 | 540 |
| 618 | 53 |
| 619 | 38 |
| 620 | >5.9E+03 |
| 621 | 4100 |
| 622 | 170 |
| 623 | 30 |
| 624 | 180 |
| 625 | 20 |
| 626 | 110 |
| 627 | 160 |
| 628 | 630 |
| 629 | 160 |
| 630 | 160 |
| 631 | 100 |
| 632 | 190 |
| 633 | 31 |
| 634 | 140 |
| 635 | 100 |
| 636 | 100 |
| 637 | 250 |
| 638 | 47 |
| 639 | 150 |
| 640 | 85 |
| 641 | 56 |
| 642 | 160 |
| 643 | 64 |
| 644 | >6.3E+03 |
| 645 | 2400 |
| 646 | 2200 |
| 647 | 130 |
| 648 | 39 |
| 649 | 170 |
| 650 | 3600 |
| 651 | 460 |
| 652 | 180 |
| 653 | 180 |
| 654 | 150 |
| 655 | 84 |
| 656 | 180 |
| 657 | 170 |
| 658 | 610 |
| 659 | 510 |
| 660 | 200 |
| 661 | 170 |
| 662 | 400 |
| 663 | 110 |
| 664 | 200 |
| 665 | 250 |
| 667 | 55 |
| 668 | 250 |
| 669 | 3200 |
| 670 | 170 |
| 671 | 130 |
| 672 | 180 |
| 673 | 240 |
| 674 | 100 |
| 675 | >1000 |
| 676 | 190 |
| 677 | 1400 |
| 678 | 350 |
| 679 | 200 |
| 680 | 75 |
| 681 | 32 |
| 682 | 83 |
| 683 | 210 |
| 684 | 59 |
| 685 | 49 |
| 686 | 460 |
| 687 | 40 |
| 688 | >4.5E+03 |
| 689 | 120 |
| 690 | 150 |
| 691 | 110 |
| 692 | 350 |
| 693 | 120 |
| 694 | 46 |
| 695 | 130 |
| 696 | 200 |
| 697 | 200 |
| 698 | 170 |
| 699 | 130 |
| 700 | 210 |
| 701 | 150 |
| 702 | 25 |
| 703 | 60 |
| 704 | 79 |
| 705 | 510 |
| 706 | 420 |
| 707 | 470 |
| 708 | 160 |
| 709 | 80 |
| 710 | 2300 |
| 711 | 74 |
| 712 | 53 |
| 713 | 82 |
| 714 | 230 |
| 715 | 230 |
| 716 | 34 |
| 717 | 30 |
| 718 | 180 |
| 719 | 14 |
| 720 | 55 |
| 721 | 140 |
| 722 | 160 |
| 723 | 81 |
| 724 | 56 |
| 725 | 210 |
| 726 | 1900 |
| 727 | 32 |
| 728 | 86 |
| 729 | >6.5E+03 |
| 730 | 260 |
| 731 | 120 |
| 732 | 49 |
| 733 | 46 |
| 734 | 81 |
| 735 | >6.2E+03 |
| 736 | 220 |
| 737 | 2100 |
| 738 | 3700 |
| 739 | 650 |
| 740 | 600 |
| 741 | 310 |
| 742 | 82 |
| 743 | 160 |
| 744 | 69 |
| 745 | 160 |
| 746 | 33 |
| 747 | 2700 |
| 748 | 92 |
| 749 | 140 |
| 750 | 160 |
| 751 | 190 |
| 752 | 190 |
| 753 | 170 |
| 754 | 340 |
| 755 | 87 |
| 756 | >6.3E+03 |
| 757 | 530 |
| 758 | 170 |
| 759 | 520 |
| 760 | 160 |
| 761 | 69 |
| 762 | 620 |
| 763 | 34 |
| 764 | >7.5E+03 |
| 765 | 86 |
| 766 | 100 |
| 767 | 130 |
| 768 | 92 |
| 769 | 120 |
| 770 | 430 |
| 771 | 140 |
| 772 | 130 |
| 773 | 190 |
| 774 | 1500 |
| 775 | 99 |
| 776 | 150 |
| 777 | 720 |
| 778 | 280 |
| 779 | 1300 |
| 780 | 120 |
| 781 | 130 |
| 782 | 120 |
| 783 | 180 |
| 784 | 290 |
| 785 | 230 |
| 786 | 120 |
| 787 | 3000 |
| 788 | 970 |
| 789 | 97 |
| 790 | 240 |
| 791 | 140 |
| 792 | 160 |
| 793 | 200 |
| 794 | 1300 |
| 795 | 130 |
| 796 | 510 |
| 797 | 170 |
| 798 | 82 |
| 799 | 79 |
| 800 | 170 |
| 801 | 140 |
| 802 | 38 |
| 803 | 860 |
| 804 | 980 |
| 805 | 820 |
| 806 | 680 |
| 807 | 600 |
| 808 | 420 |
| 809 | 200 |
| 810 | 170 |
| 811 | 120 |
| 812 | 51 |
| 813 | 550 |
| 814 | 430 |
| 815 | 69 |
| 816 | 140 |
| 817 | 3600 |
| 818 | 1300 |
| 819 | 1000 |
| 820 | 220 |
| 821 | 67 |
| 822 | 390 |
| 823 | 130 |
| 824 | 130 |
| 825 | 1000 |
| 826 | 4400 |
| 827 | 910 |
| 828 | 1000 |
| 829 | 130 |
| 830 | 210 |
| 831 | 440 |
| 832 | 120 |
| 833 | 120 |
| 834 | 140 |
| 836 | 89 |
| 837 | 34 |
| 839 | 1500 |
| 840 | 1200 |
| 841 | 75 |
| 842 | 270 |
| 843 | 2200 |
| 844 | 600 |
| 845 | 69 |
| 846 | 420 |
| 847 | 2000 |
| 848 | 260 |
| 849 | 2600 |
| 850 | 52 |
| 851 | 61 |
| 852 | 56 |
| 853 | 55 |
| 854 | 80 |
| 855 | 420 |
| 856 | 260 |
| 857 | 480 |
| 858 | 55 |
| 859 | 100 |
| 860 | 48 |
| 861 | 620 |
| 862 | 220 |
| 863 | 29 |
| 864 | 130 |
| 865 | 150 |
| 866 | 52 |
| 867 | 970 |
| 868 | 760 |
| 869 | 1400 |
| 870 | 1900 |
| 871 | 720 |
| 872 | 2100 |
| 873 | >5.2E+03 |
| 874 | 4000 |
| 875 | 86 |
| 876 | 290 |
| 877 | 790 |
| 878 | 560 |
| 879 | 110 |
| 880 | 180 |
| 881 | 150 |
| 882 | 180 |
| 883 | 160 |
| 884 | 57 |
| 885 | 110 |
| 886 | 180 |
| 887 | >6.3E+03 |
| 888 | 26 |
| 889 | 41 |
| 890 | 60 |
| 891 | 44 |
| 892 | 51 |
| 893 | 40 |
| 894 | 130 |
| 895 | 53 |
| 896 | 46 |
| 897 | 140 |
| 898 | 120 |
| 899 | 150 |
| 900 | 70 |
| 901 | 48 |
| 902 | 383 |
| 903 | 45 |
| 904 | 75 |
| 905 | 62 |
| 906 | 48 |
| 907 | 130 |
| 908 | 77 |
| 909 | 62 |
| 910 | 97 |
| 911 | 140 |
| 912 | 170 |
| 913 | 130 |
| 914 | 130 |
| 915 | 86 |
| 916 | 570 |
| 917 | 110 |
| 918 | 170 |
| 919 | 160 |
| 920 | 84 |
| 921 | 52 |
| 922 | 420 |
| 923 | >1000 |
| 924 | 85 |
| 925 | 32 |
| 926 | 75 |
| 927 | 200 |
| 928 | 250 |
| 929 | 110 |
| 930 | 400 |
| 931 | 170 |
| 932 | 20 |
| 933 | 180 |
| 934 | 120 |
| 935 | 63 |
| 936 | 24 |
| 937 | 50 |
| 938 | 290 |
| 939 | 69 |
| 940 | 34 |
| 941 | 39 |
| 942 | 88 |
| 943 | 80 |
| 944 | 46 |
| 945 | 47 |
| 946 | 130 |
| 947 | 35 |
| 948 | 150 |
| 949 | 250 |
| 950 | 51 |
| 951 | 100 |
| 952 | 130 |
| 953 | 100 |
| 954 | 31 |
| 955 | 26 |
| 956 | 360 |
| 957 | 23 |
| 958 | 47 |
| 959 | 190 |
| 960 | 110 |
| 961 | 38 |
| 962 | 15 |
| 963 | 12 |
| 964 | 64 |
| 965 | 130 |
| 966 | 32 |
| 967 | 48 |
| 968 | 40 |
| 969 | 45 |
| 970 | 34 |
| 971 | 107 |
| 972 | 213 |

The foregoing disclosure has been described in some detail by way of illustration and example, for purposes of clarity and understanding. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

What is claimed is:

1. A method of modulating c-MYC activity comprising administering an effective amount of a compound of Formula I, or a pharmaceutically acceptable salt thereof, 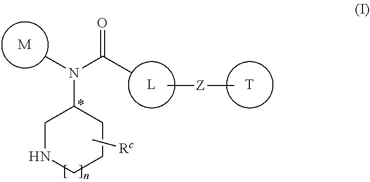
wherein
* denotes a stereocenter;
M is C₁₋₉ heteroaryl, C₁₋₉ heterocyclyl, each optionally substituted with 1, 2 or 3 substituents independently chosen from halo, substituted or unsubstituted alkyl, substituted or unsubstituted haloalkyl, substituted or unsubstituted alkenyl, substituted or unsubstituted alkynyl, substituted or unsubstituted ester, carboxylic acid, substituted or unsubstituted acetyl, substituted or unsubstituted amido, substituted or unsubstituted carbamate, substituted or unsubstituted amino, substituted or unsubstituted alkoxy, substituted or unsubstituted alkoxycarbonyl, substituted or unsubstituted thioether, cyano, substituted or unsubstituted aryl, substituted or unsubstituted heteroaryl, substituted or unsubstituted cycloalkyl, substituted or unsubstituted heterocyclyl, and combinations thereof, wherein each substituted group has 1, 2 or 3 substituents;
R^{c} is absent, C₁-C₆ alkyl, OH, NH₂, fluoro or gem-difluoro;
n is 0, 1 or 2;
L is a bond, C₆₋₁₀ arylene, C₃₋₉ heteroarylene, C₃₋₁₀ cycloalkylene, C₃₋₁₀ cycloalkenylene, or C₁₋₉ heterocyclylene, each optionally substituted with 1, 2 or 3 substituents independently chosen from halo, aryl, heteroaryl, alkyl, alkenyl, alkynyl, cycloalkyl, heterocyclyl, haloalkyl, carboxylic acid, ester, amide, alkoxy, alkoxyaryl, alkoxyheteroaryl, thioether, amino, NH-aryl, NH-heteroaryl, and cyano;
Z is a bond, —O—, S, C₁₋₃ alkylene, —(CH₂)₂C(O)NH—, —N(R^{b})—, —N(R^{b})(C₁₋₃ alkylene), where R^{b} is H, or C₁₋₃ alkyl; and
T is absent, C₆₋₁₀ aryl, C₃₋₁₀ cycloalkyl, C₂₋₉ heteroaryl, C₁₋₉ heterocyclyl, each optionally substituted with 1, 2 or 3 substituents independently chosen from OH, halo, cyano, substituted or unsubstituted aryl, substituted or unsubstituted heteroaryl, substituted or unsubstituted alkyl, haloalkyl, substituted or unsubstituted alkenyl, substituted or unsubstituted alkynyl, substituted or unsubstituted cycloalkyl, substituted or unsubstituted heterocyclyl, substituted or unsubstituted alkoxycarbonyl, hydroxycarbonyl, substituted or unsubstituted ester, substituted or unsubstituted amido, substituted or unsubstituted alkoxy, substituted or unsubstituted alkoxyaryl, substituted or unsubstituted alkoxyheteroaryl, substituted or unsubstituted thioether, substituted or unsubstituted amino, NH-aryl, and NH-heteroaryl, wherein each substituted group has 1, 2 or 3 substituents;
provided that when L is a bond, T is not absent and when T is absent, then L is not a bond.

2. The method of claim 1, wherein the MYC activity is modulated in a cell.

3. The method of claim 1, wherein the MYC activity is modulated in a cellular assay.

4. The method of claim 1, wherein the MYC activity is modulated in an In Vitro Multiple Point Translation Screening Assay.

5. The method of claim 1, wherein the MYC activity is modulated in a subject in need thereof, wherein the subject is a model animal or a human.

6. The method of claim 1, wherein the MYC activity modulates transcriptional regulation of a gene that is a biomarker.

7. The method of claim 1, wherein the MYC activity modulates transcriptional regulation of a gene that is a biomarker that predicts anti-MYC activity.

8. The method of anyone of claims 1-7, wherein the compound of Formula I inhibits MYC activity.

9. The method of any one of claims 1 to 7, wherein the compound of Formula I activates MYC activity.

10. A method of treating a condition associated with MYC activity in a subject in need thereof comprising administering to the subject an effective amount of a compound of Formula I, or a pharmaceutically acceptable salt thereof, 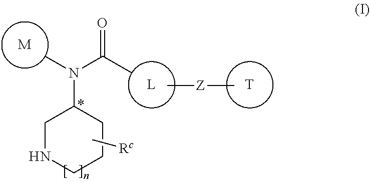
wherein
* denotes a stereocenter;
M is C₁₋₉ heteroaryl, C₁₋₉ heterocyclyl, each optionally substituted with 1, 2 or 3 substituents independently chosen from halo, substituted or unsubstituted alkyl, substituted or unsubstituted haloalkyl, substituted or unsubstituted alkenyl, substituted or unsubstituted alkynyl, substituted or unsubstituted ester, carboxylic acid, substituted or unsubstituted acetyl, substituted or unsubstituted amido, substituted or unsubstituted carbamate, substituted or unsubstituted amino, substituted or unsubstituted alkoxy, substituted or unsubstituted alkoxycarbonyl, substituted or unsubstituted thioether, cyano, substituted or unsubstituted aryl, substituted or unsubstituted heteroaryl, substituted or unsubstituted cycloalkyl, substituted or unsubstituted heterocyclyl, and combinations thereof, wherein each substituted group has 1, 2 or 3 substituents;
R^{c} is H, C₁-C₆ alkyl, OH, NH₂, fluoro or gem-difluoro;
n is 0, 1 or 2;
L is a bond, C₆₋₁₀ arylene, C₃₋₉ heteroarylene, C₃₋₁₀ cycloalkylene, C₃₋₁₀ cycloalkenylene, or C₁₋₉ heterocyclylene, each optionally substituted with 1, 2 or 3 substituents chosen from halo, aryl, heteroaryl, alkyl, alkenyl, alkynyl, cycloalkyl, heterocyclyl, haloalkyl, carboxylic acid, ester, amide, alkoxy, alkoxyaryl, alkoxyheteroaryl, thioether, amino, NH-aryl, NH-heteroaryl, and cyano;
Z is a bond, —O—, —S—, C₁₋₃ alkylene, —(CH₂)₂C(O)NH—, —N(R^{b})—, —N(R^{b})(C₁₋₃ alkylene), where R^{b} is H, or C₁₋₃ alkyl; and
T is absent, C₆₋₁₀ aryl, C₃₋₁₀ cycloalkyl, C₂₋₉ heteroaryl, C₁₋₉ heterocyclyl, each optionally substituted with 1, 2 or 3 substituents independently chosen from OH, halo, cyano, substituted or unsubstituted aryl, substituted or unsubstituted heteroaryl, substituted or unsubstituted alkyl, haloalkyl, substituted or unsubstituted alkenyl, substituted or unsubstituted alkynyl, substituted or unsubstituted cycloalkyl, substituted or unsubstituted heterocyclyl, substituted or unsubstituted alkoxycarbonyl, hydroxycarbonyl, substituted or unsubstituted ester, substituted or unsubstituted amido, substituted or unsubstituted alkoxy, substituted or unsubstituted alkoxyaryl, substituted or unsubstituted alkoxyheteroaryl, substituted or unsubstituted thioether, substituted or unsubstituted amino, NH-aryl, and NH-heteroaryl, wherein each substituted group has 1, 2 or 3 substituents;
provided that when L is a bond, T is not absent and when T is absent, then L is not a bond.

11. The method of claim 10, wherein the condition associated with MYC activity is cancer.

12. The method of claim 11, wherein the cancer is chosen from solid and hematopoietic cancers such as breast, prostate, pancreatic, hepatocellular, renal, multiple myeloma, lymphoma, lung, skin, oral squamous, haemopoietic, and neuroblastoma.

13. The method of claim 11, wherein the cancer is derived from breast, colon, lung or hematopoietic lineages.

14. The method of any one of claims 11 to 13, wherein the method further comprises administering to the subject an effective amount of a second anti-cancer agent.

15. The method of claim 14, wherein the second anti-cancer agent is chosen from PARP inhibitors, immune oncology (I/O) drugs, tyrosine kinase inhibitors, CHK1 inhibitor, GLS inhibitor, SAE1/2 modulators, BUD31 modulators, TRRAP modulators and BRD4 modulators.

16. The method of claim 14, wherein the second anti-cancer agent is chosen from Olaparib (Lynparza), rucaparib (Rubraca), niraparib (Zejula), nivolumab (Opdivo), atezolizumab (Tecentriq), pembrolizumab (Keytruda), avelumab (Bavencio), durvalumab (Imfinzi) and cemiplimab-rwlc (Libtayo), axitinib (Inlyta) dasatinib (Sprycel), erlotinib (Tarceva), imatinib (Glivec), nilotinib (Tasigna), pazopanib (Votrient), sunitinib (Sutent).

17. A method of treating cancer in a subject in need thereof comprising administering to the subject an effective amount of a compound of Formula I, or a pharmaceutically acceptable salt thereof, 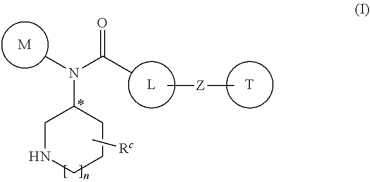
wherein
* denotes a stereocenter;
M is C₁₋₉ heteroaryl, C₁₋₉ heterocyclyl, each optionally substituted with 1, 2 or 3 substituents independently chosen from halo, substituted or unsubstituted alkyl, substituted or unsubstituted haloalkyl, substituted or unsubstituted alkenyl, substituted or unsubstituted alkynyl, substituted or unsubstituted ester, carboxylic acid, substituted or unsubstituted acetyl, substituted or unsubstituted amido, substituted or unsubstituted carbamate, substituted or unsubstituted amino, substituted or unsubstituted alkoxy, substituted or unsubstituted alkoxycarbonyl, substituted or unsubstituted thioether, cyano, substituted or unsubstituted aryl, substituted or unsubstituted heteroaryl, substituted or unsubstituted cycloalkyl, substituted or unsubstituted heterocyclyl, and combinations thereof, wherein each substituted group has 1, 2 or 3 substituents;
R^{c} is H, C₁-C₆ alkyl, OH, NH₂, fluoro or gem-difluoro;
n is 0, 1 or 2;
L is a bond, C₆₋₁₀ arylene, C₃₋₉ heteroarylene, C₃₋₁₀ cycloalkylene, C₃₋₁₀ cycloalkenylene, or C₁₋₉ heterocyclylene, each optionally substituted with 1, 2 or 3 substituents independently chosen from halo, aryl, heteroaryl, alkyl, alkenyl, alkynyl, cycloalkyl, heterocyclyl, haloalkyl, carboxylic acid, ester, amide, alkoxy, alkoxyaryl, alkoxyheteroaryl, thioether, amino, NH-aryl, NH-heteroaryl, and cyano;
Z is a bond, —O—, —S—, C₁₋₃ alkylene, —(CH₂)₂C(O)NH—, —N(R^{b})—, —N(R^{b})C₁₋₃ alkylene), where R^{b} is H, or C₁₋₃ alkyl; and
T is absent, C₆₋₁₀ aryl, C₃₋₁₀ cycloalkyl, C₂₋₉ heteroaryl, C₁₋₉ heterocyclyl, each optionally substituted with 1, 2 or 3 substituents independently chosen from OH, halo, cyano, substituted or unsubstituted aryl, substituted or unsubstituted heteroaryl, substituted or unsubstituted alkyl, haloalkyl, substituted or unsubstituted alkenyl, substituted or unsubstituted alkynyl, substituted or unsubstituted cycloalkyl, substituted or unsubstituted heterocyclyl, substituted or unsubstituted alkoxycarbonyl, hydroxycarbonyl, substituted or unsubstituted ester, substituted or unsubstituted amido, substituted or unsubstituted alkoxy, substituted or unsubstituted alkoxyaryl, substituted or unsubstituted alkoxyheteroaryl, substituted or unsubstituted thioether, substituted or unsubstituted amino, NH-aryl, and NH-heteroaryl, wherein each substituted group has 1, 2 or 3 substituents;
provided that when L is a bond, T is not absent and when T is absent, then L is not a bond.

18. The method of claim 17, wherein the cancer is chosen from solid and hematopoietic cancers such as breast, prostate, pancreatic, hepatocellular, renal, multiple myeloma, lymphoma, lung, skin, oral squamous, haemopoietic, and neuroblastoma.

19. The method of claim 17, wherein the cancer is derived from breast, colon, lung or hematopoietic lineages.

20. The method of any one of claims 17 to 19, wherein the method further comprises administering to the subject an effective amount of a second anti-cancer agent.

21. The method of claim 20, wherein the second anti-cancer agent is chosen from PARP inhibitors, immune oncology (I/O) drugs, tyrosine kinase inhibitors, CHK1 inhibitor, GLS inhibitor, SAE1/2 modulators, BUD31 modulators, TRRAP modulators and BRD4 modulators.

22. The method of claim 20, wherein the second anti-cancer agent is chosen from Olaparib (Lynparza), rucaparib (Rubraca), niraparib (Zejula), nivolumab (Opdivo), atezolizumab (Tecentriq), pembrolizumab (Keytruda), avelumab (Bavencio), durvalumab (Imfinzi) and cemiplimab-rwlc (Libtayo), axitinib (Inlyta) dasatinib (Sprycel), erlotinib (Tarceva), imatinib (Glivec), nilotinib (Tasigna), pazopanib (Votrient), sunitinib (Sutent).

23. The method of any one of claims 1 to 22, wherein M is pyrrolo[2,3-c] pyridinyl, pyrrolo[3,2-c] pyridinyl, pyrazolo[3,4-c]pyridinyl, thieno[2,3-c]pyridinyl, thieno[3,2-c]pyridinyl, quinolinyl, isoquinolinyl, quinolinyl-2-one, 1,6-naphthyridinyl, 1,6-naphthyridinyl, pyridinyl, or 2-pyrazinyl, each optionally substituted with 1, 2 or 3 substituents independently chosen from halo, substituted or unsubstituted alkyl, substituted or unsubstituted haloalkyl, substituted or unsubstituted alkenyl, substituted or unsubstituted alkynyl, ester, carboxylic acid, substituted or unsubstituted amido, substituted or unsubstituted amino, substituted or unsubstituted alkoxy, substituted or unsubstituted thioether, cyano, substituted or unsubstituted aryl, substituted or unsubstituted heteroaryl, substituted or unsubstituted cycloalkyl, substituted or unsubstituted heterocyclyl, and combinations thereof, wherein each substituted group has 1, 2 or 3 substituents.

24. The method of any one of claims 1 to 22, wherein M is 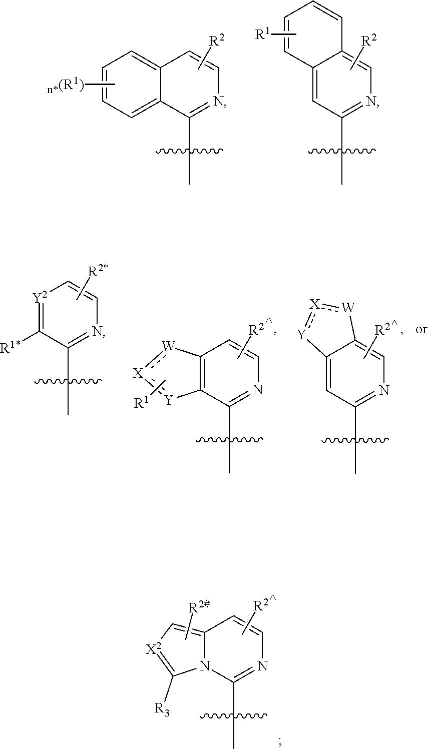
wherein
each R¹ is independently absent halo, substituted or unsubstituted alkyl, substituted or unsubstituted haloalkyl, substituted or unsubstituted alkenyl, substituted or unsubstituted alkynyl, substituted or unsubstituted aryl, substituted or unsubstituted ester, substituted or unsubstituted acetyl, carboxylic acid, substituted or unsubstituted alkoxycarbonyl, substituted or unsubstituted amido, substituted or unsubstituted carbamate; substituted or unsubstituted alkoxy, substituted or unsubstituted thioether, substituted or unsubstituted amino, cyano, substituted or unsubstituted heteroaryl, substituted or unsubstituted cycloalkyl or substituted or unsubstituted heterocyclyl, wherein each substituted group has 1, 2 or 3 substituents;
n* is 0, 1, 2 or 3;
R² is absent, halo, alkyl, alkenyl, alkynyl, aryl heteroaryl or cyano;
R^{1*} is absent, halo, alkyl, alkenyl, alkynyl, aryl, ester, carboxylic acid, amide, alkoxy, thioether, cyano, heteroaryl, cycloalkyl or heterocyclyl;
R^{2*} is absent, halo, alkyl, alkenyl, alkynyl, aryl, ester, carboxylic acid, amide, alkoxy, thioether, cyano, heteroaryl, cycloalkyl or heterocyclyl; and
each W, X, and Y are independently —CH, —N, —NH, N-alkyl, O, or S;
R^{2#} is absent, halo, alkyl, alkenyl, alkynyl, aryl, ester, carboxylic acid, amide, alkoxy, thioether, amino, cyano, heteroaryl, cycloalkyl or heterocyclyl;
X² is CH, N, C-alkyl, C-alkenyl, C-alkynyl, C-aryl or C-heteroaryl;
Y² is CH or N;
R^{2\hat{ }} is absent, alkyl, alkenyl, alkynyl, cyano or halo; and
R₃ is absent, alkyl, alkenyl, alkynyl, halo, cyano, ester, carboxylic acid, amide, aryl, heteroaryl, alkoxy, thioether, or amino; and [symbol]
is a bond that is a double bond or a single bond.

25. The method of any one of claims 1 to 22, wherein M is 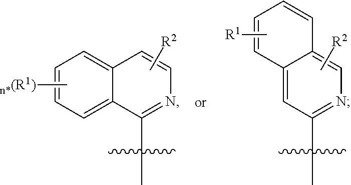
wherein
each R¹ is independently absent, cyano; chloro; bromo; fluoro; acetyl; amido; carbamate; alkylcarbamate; pyrazinyl; tetrazolyl; hydroxycarbonyl; alkoxycarbonyl; pyridinyl-2-one; C₁₋₆ alkoxy; C₁₋₆ haloalkyl; C₁₋₆ cycloalkyl; C₁₋₆ alkyl optionally substituted with OH, pyridinyl, amido, or phenyl; C₂₋₆ alkenyl optionally substituted with amido, OH, pyrazolyl, pyridinyl, isoxazolyl, thiophenyl, alkyl substituted amido, hydroxyalkylamido, arylalkylamido, alkoxycarbonyl, or hydroxycarbonyl; C₂₋₆ alkynyl optionally substituted with OH; phenyl optionally substituted with OH, halo, hydroxyalkyl, sulfonyl, or alkylsulfonyl; pyrazolyl optionally substituted with C₁₋₆ alkyl, C₁₋₆ hydroxyalkyl, or acetamide; pyridinyl optionally substituted with amino; triazolyl optionally substituted with C₁₋₆ hydroxyalkyl; thiazolyl optionally substituted with C₁₋₆ alkyl; or 1,3,4-thiadiazolyl optionally substituted with C₁₋₆ alkyl;
n* is 0, 1 or 2;
R² is absent, halo, or C₁₋₆ alkyl, or cyano.

26. The method of any one of claims 1 to 22, wherein M is 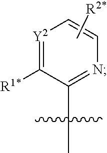
wherein
Y² is CH or N;
R^{1*} is absent; chloro; cyano; amido; alkylamido; allyl; alkoxycarbonyl; pyrazolyl; methylpyrazolyl; hydroxyethylpyrazolyl; isoxazolyl; 2,5-dihydro-pyrrolyl; thiophenyl; thieno[3,2-c]pyridinyl; C₁₋₆ alkyl; C₂₋₆ alkenyl optionally substituted with OH; or C₂₋₆ alkynyl optionally substituted with OH;
R^{2*} is absent; C₁₋₆ alkyl; halo; pyrazolyl; C₂₋₆ alkenyl optionally substituted with phenyl, methylpyrazolyl, or pyridinyl.

27. The method of any one of claims 1 to 22, wherein M is 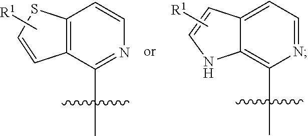
wherein
R¹ is absent, bromo, fluoro; chloro; amido; alkyl substituted amido; cyano; C₁₋₆ cycloalkyl; C₁₋₆ alkyl optionally substituted with phenyl or pyridinyl; C₂₋₆ alkenyl optionally substituted with OH, amido, alkyl substituted amido, or alkoxycarbonyl; C₂₋₆ alkynyl optionally substituted with OH, alkyl substituted amido, or pyridinyl; phenyl optionally substituted with one, two or three groups chosen from halo, OH, hydroxyalkyl, halohydroxyalkyl, cyano, CH₃O—, carboxyl, amino, amido, amidoalkyl, amidoalkoxy, alkyl substituted amido, phenyl substituted amido, hydroxyalkylamido, acetamido, aldehyde, acetyl, hydroxyalkylamido, sulfonylamino, alkylsulfonyl, isoxazolyl, pyrazolyl, and thiourea; pyridinyl optionally substituted with one or two groups chosen from C₁₋₆ alkyl, halo, amino, cyano, cyanoalkyl, haloalkyl, and alkoxy; 1,2,3-triazolyl optionally substituted with C₁₋₆ alkyl; thiazolyl optionally substituted with C₁₋₆ alkyl, hydroxyalkyl, alkoxycarbonyl, or hydroxycarbonyl; pyrazolyl optionally substituted with C₁₋₆ alkyl, hydroxyalkyl, pyridinyl, methylpyridinyl, or acetamido; benzo[d]isoxazolyl optionally substituted with amino; thiophenyl optionally substituted with hydroxyalkyl, cyano, or amido; pyrimidinyl optionally substituted with amino or alkylamino; pyridylcarbonyl; [1,2,4]triazolo[1,5-a]pyridinyl; pyrazinyl; pyridazinyl; pyridinyl-2-one; isoindolinyl-1-one; indolinyl; indolinyl-2-one; 1,3-dihydro-2H-benzo[d]imidazolyl-2-one; or indazolyl, wherein each substituted group has 1, 2 or 3 substituents.

28. The method of any one of claims 1 to 22, wherein M is 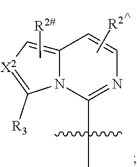
wherein
X² is CH, N, C-alkyl, C-alkenyl, C-alkynyl, C-aryl or C-heteroaryl;
R^{2#} is absent, halo, alkyl, alkenyl, alkynyl, aryl, ester, carboxylic acid, amido, alkoxy, thioether, amino, cyano, heteroaryl, cycloalkyl or heterocyclyl;
R^{2\hat{ }} is absent, alkyl, alkenyl, alkynyl, cyano or halo; and
R³ is H, alkyl, alkenyl, alkynyl, halo, cyano, ester, carboxylic acid, amide, aryl, heteroaryl, alkoxy, thioether, or amino.

29. The method of any one of claims 1 to 28, wherein L is a bond, phenylene, pyridinylene, piperidinylene, or piperazinylene, each optionally substituted with 1 or 2 substituents chosen from halo, C₁₋₃ alkyl, C₁₋₃ alkyloxy and combinations thereof, or —NR^{a}—, where R^{a} is H or C₁₋₃ alkyl.

30. The method of any one of claims 1 to 28, wherein L is a bond.

31. The method of any one of claims 1 to 28, wherein L is phenylene, pyridinylene, piperidinylene, or piperazinylene, each optionally substituted with 1 or 2 substituents chosen from halo, C₁₋₃ alkyl, C₁₋₃ alkyloxy and combinations thereof.

32. The method of any one of claims 1 to 28, wherein L is chosen from phenylene, fluorophenylene, chlorophenylene, methoxyphenylene, methylphenylene, ethylphenylene, pyridinylene, piperidinylene, and piperazinylene.

33. The method of any one of claims 1 to 32, wherein Z is a bond.

34. The method of any one of claims 1 to 32, wherein Z is C₁₋₃ alkylene.

35. The method of any one of claims 1 to 32, wherein Z is methylene.

36. The method of any one of claims 1 to 32, wherein Z is —NH.

37. The method of any one of claims 1 to 32, wherein Z is —N(CH₃)—.

38. The method of any one of claims 1 to 37, wherein T is a substituted or unsubstituted group chosen from [1,2,3]triazolo[4,5-b]pyridinyl, pyrazolo[2,3-a]pyrimidinyl, 5,6-dihydropyrrolo[2,3-d]pyrimidinyl, pyrazolo[3,4-d]pyrimidinyl, phenyl, purinyl, pyrimidinyl, pyridinyl, pyrazinyl, pyridazinyl, pyrazolyl, tetrazolyl, 1,2,4-triazolyl, 1,2,3-triazolyl, imidazolyl, benzo[d]imidazolyl, pyrrolyl, pyrrolidinyl, isoxazolyl, isoindolyl, piperidinyl, oxetanyl, thiophenyl, morpholino, thiazolyl, pyridinyl-2-one, pyrazinyl-2-one, indazolyl, 4,5,6,7-tetrahydro-indazolyl, isoindolinyl-1,3-dione, 1,3,4-oxadiazolyl, 1,3,4-oxadiazolyl-2-one, benzo[d]isoxazolyl, benzo[d]isothiazolyl, 1,2,4-oxadiazolyl, 1,3,4-thiadiazolyl, 1,3-dihydro-2H-imidazolyl-2-one, phthalazinyl, isoquinolinyl, 2λ⁵-quinolinyl-2-one, 6,7-dihydro-4H-[1,2,3]triazolo[5,1-c][1,4]oxazinyl, 2,3-dihydro-4λ²-pyrido[3,2-b][1,4]oxazinyl, thiazolo[5,4-c]pyridinyl, and quinazolinyl.

39. The method of claim 38, wherein T is [1,2,3]triazolo[4,5-b]pyridinyl optionally substituted with halo, haloalkyl, cyano, methyl, methoxy, ethyl, pyrazolyl, alkoxycarbonyl, hydroxycarbonyl, amido, or phenyl; 5,6-dihydropyrrolo[2,3-d]pyrimidinyl optionally substituted with methyl; isoxazolyl optionally substituted with methyl, haloalkyl, alkyoxycarbonyl, or amido; thiazolyl optionally substituted with methyl; pyrazolyl optionally substituted with C₁₋₆ alkyl, C₁₋₆ hydroxyalkyl, C₁₋₆ alkoxyalkyl, amino, phenyl, hydroxycarbonyl or any combination of two or three thereof; pyrimidinyl optionally substituted with amido, amino, or one or two methyl groups; 1,2,3-triazolyl optionally substituted with C₁₋₆ alkyl, C₁₋₆ hydroxyalkyl, C₁₋₆ alkoxyalkyl, halo, or any combination of two or three thereof; 1,2,4-triazolyl optionally substituted with methyl; phenyl optionally substituted with OH, pyrazolyl, pyridinyl, cyano, indazolyl, amidoindazolyl, methoxy, benzyloxy, C₂₋₆ alkynyl, or any combination of two or three thereof, and wherein the C₂₋₆ alkynyl is further substituted by OH, oxo, alkoxy, isoindolinyl-1,3-dione, morpholino, amino, hydroxyalkyl substituted amino, pyrrolidinyl substituted amino, hydroxypyrrolidinyl substituted amino, pyridinyl substituted amino, pyridinylmethyl substituted amino, or any combination of two or three thereof; pyridinyl optionally substituted with halo, methyl, amino pyridinyl or any combination of two or three thereof; pyrazolo[3,4-d]pyrimidinyl optionally substituted with phenoxyphenyl; imidazolyl optionally substituted phenyl, methyl, amino, or any combination of two or three thereof; pyrazinyl optionally substituted with OH, halo, cyano, amino, substituted amino, C₁₋₆ alkoxycarbonyl, C₁₋₆ alkyl, C₁₋₆ hydroxyalkyl, C₁₋₆ alkoxyalkyl, C₁₋₈ cycloalkyl, substituted C₁₋₈ cycloalkyl, C₁₋₃ alkyl substituted with cycloalkyl, C₁₋₃ alkyl substituted with substituted cycloalkyl, heterocyclyl, substituted heterocyclyl, C₁₋₃ alkyl substituted with heterocyclyl, C₁₋₃ alkyl substituted with substituted heterocyclyl, heteroaryl, substituted heteroaryl, C₁₋₃ alkyl substituted with heteroaryl, C₁₋₃ alkyl substituted with substituted heteroaryl, aryl, substituted aryl, C₁₋₃ alkyl substituted with aryl, C₁₋₃ alkyl substituted with substituted aryl, C₂₋₆ alkenyl, C₂₋₆ hydroxyalkenyl, C₂₋₆ alkoxyalkenyl, C₂₋₆ alkynyl, C₂₋₆ hydroxyalkynyl, C₂₋₆ alkoxyalkynyl or any combination of two or three thereof; tetrazolyl optionally substituted with methyl, ethyl, or haloalkyl; 1,3,4-oxadiazolyl optionally substituted with C₁₋₆ alkyl, C₃₋₆ cycloalkyl; 1,3,4-thiadiazolyl optionally substituted with C₁₋₆ alkyl, C₁₋₆ hydroxyalkyl, C₁₋₆ alkoxyalkyl, C₃₋₆ cycloalkyl, CD₃, OH; pyridinyl-2-one optionally substituted with methyl; 1,3-dihydro-2H-imidazolyl-2-one optionally substituted with methyl; 1,3,4-oxadiazolyl-2-one optionally substituted with methyl; pyrrolidinyl optionally substituted with methyl; benzo[d]isothiazolyl optionally substituted with methyl; 1,2,4-oxadiazolyl optionally substituted with C₁₋₆ alkyl, cyclopropyl or phenyl; morpholino optionally substituted with 1 or 2 C₁₋₆ alkyl groups; or pyridazinyl optionally substituted with halo, wherein each substituted group has 1, 2 or 3 substituents.

40. The method of any one of claims 1 to 39, wherein the * stereocenter is in the (R) configuration.

41. The method of any one of claims 1 to 39, wherein the compound is selected from Table 1, and/or a pharmaceutically acceptable.

42. The method of any one of claims 1 to 33, wherein the compound is selected from 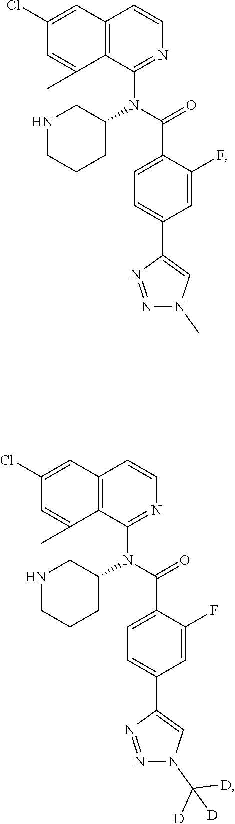 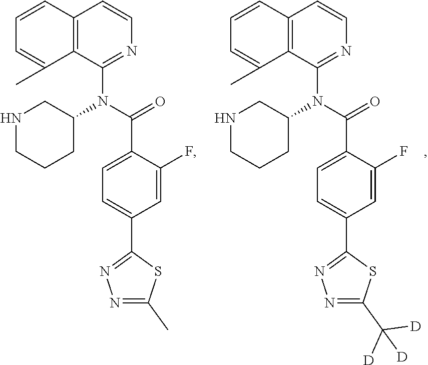 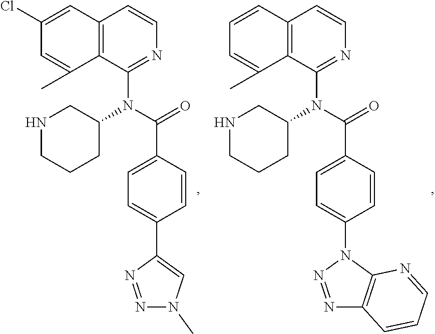 [SMILES: Cc1cc(Cl)cc2ccnc(N(C(=O)c3ccc(-c4cn(C)nn4)cc3)[C@@H]3CCCNC3)c12 ; Cc1cccc2ccnc(N(C(=O)c3ccc(-n4nnc5cccnc54)cc3)[C@@H]3CCCNC3)c12] 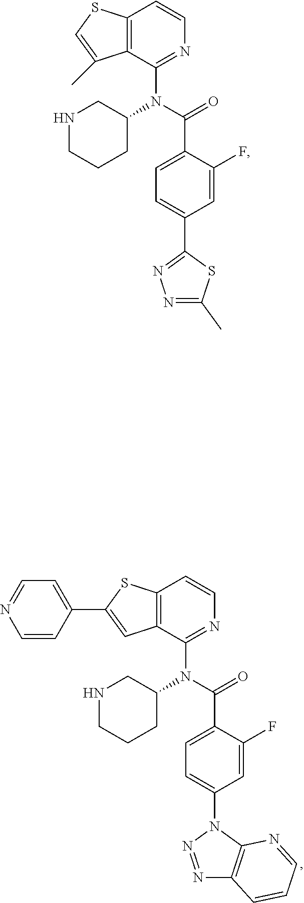 [SMILES: Cc1nnc(-c2ccc(C(=O)N(c3nccc4scc(C)c34)[C@@H]3CCCNC3)c(F)c2)s1 ; O=C(c1ccc(-n2nnc3cccnc32)cc1F)N(c1nccc2sc(-c3ccncc3)cc12)[C@@H]1CCCNC1]
and pharmaceutically acceptable salts thereof.

43. The method of any one of claims 1 to 33, wherein the compound is selected from N-(6-chloro-8-methyl-1-isoquinolyl)-2-fluoro-4-(1-methyltriazol-4-yl)-N-[(3R)-3-piperidyl]benzamide;
(R)—N-(6-chloro-8-methylisoquinolin-1-yl)-2-fluoro-4-(1-(methyl-d3)-1H-1,2,3-triazol-4-yl)-N-(piperidin-3-yl)benzamide;
(R)-2-fluoro-4-(5-methyl-1,3,4-thiadiazol-2-yl)-N-(8-methylisoquinolin-1-yl)-N-(piperidin-3-yl)benzamide;
(R)-2-fluoro-4-(5-(methyl-d3)-1,3,4-thiadiazol-2-yl)-N-(8-methylisoquinolin-1-yl)-N-(piperidin-3-yl)benzamide;
(R)—N-(6-chloro-8-methylisoquinolin-1-yl)-4-(1-methyl-1H-1,2,3-triazol-4-yl)-N-(piperidin-3-yl)benzamide;
(R)-4-(3H-[1,2,3]triazolo[4,5-b]pyridin-3-yl)-N-(8-methylisoquinolin-1-yl)-N-(piperidin-3-yl)benzamide;
(R)-2-fluoro-4-(5-methyl-1,3,4-thiadiazol-2-yl)-N-(3-methylthieno[3,2-c]pyridin-4-yl)-N-(piperidin-3-yl)benzamide;
(R)-4-(3H-[1,2,3]triazolo[4,5-b]pyridin-3-yl)-2-fluoro-N-(piperidin-3-yl)-N-(2-(pyridin-4-yl)thieno[3,2-c]pyridin-4-yl)benzamide; and pharmaceutically acceptable salts thereof.

44. The method of any one of claims 1 to 43, wherein the compound of Formula (I) is provided in a pharmaceutical composition comprising a compound and/or or a pharmaceutically acceptable salt thereof and a pharmaceutically acceptable excipient.

45. A method of treating cancer by administering to a patient in need thereof a therapeutically effective amount of a compound that selectively inhibits the translation of c-MYC mRNA to c-MYC protein.

46. The method of claim 45, wherein translation stalls by targeting a cytosolic nascent chain/ribosome complex.

47. The method of claim 46, wherein a ternary complex between small molecule, ribosome and nascent c-MYC polypeptide is visible by Cryo-electron microscopy.

48. The method of claim 45, wherein translation is stalled to deliver 50% partial inhibition of translation.

49. The method of claim 46, wherein translation is stalled resulting in 90% partial inhibition of translation.

50. The method of claim 45, wherein the compound has an IC50 below about 50 μM in an In Vitro Multiple Point Translation Screening Assay.

51. The method of claim 45, wherein the compound has an IC50 below about 5 μM in an In Vitro Multiple Point Translation Screening Assay.

52. The method of claim 45, wherein the selective inhibition of MYC is such that less than 1% of non-MYC proteins are inhibited in a Global Proteomic Assay.

53. The method of claim 45, wherein the selective inhibition of MYC is such that less than 0.1% of non-MYC proteins are inhibited in a Global Proteomic Assay.

54. The method of claim 44, wherein the compound is administered by oral administration.

55. The method of claim 44, wherein the compound has a MW of about 300 to about 600 Da.

56. A compound of Formula (Ia), (Ib), (Ic), or a pharmaceutically acceptable salt thereof: 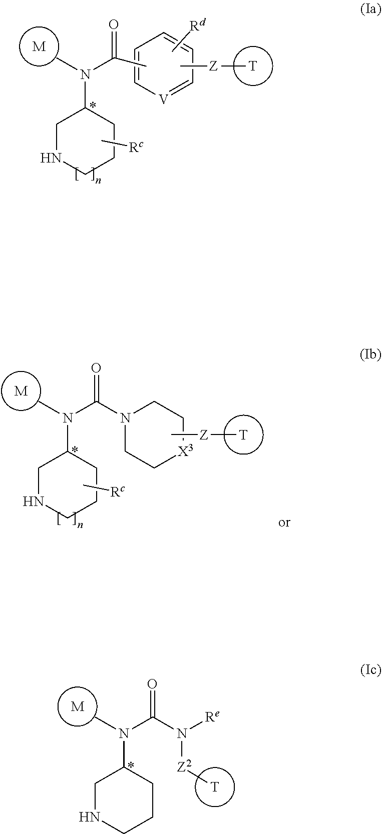
wherein
* denotes a stereocenter;
M is pyrrolo[2,3-c] pyridinyl, pyrrolo[3,2-c] pyridinyl, pyrazolo[3,4-c]pyridinyl, thieno[2,3-c]pyridinyl, thieno[3,2-c]pyridinyl, quinolinyl, isoquinolinyl, quinolinyl-2-one, 1,6-naphthyridinyl, 1,6-naphthyridinyl, pyridinyl, 2-pyrazinyl, thiazolo[5,4-c]pyridinyl, or thiazolyl, each optionally substituted with 1, 2 or 3 substituents independently chosen from halo, substituted or unsubstituted alkyl, substituted or unsubstituted haloalkyl, substituted or unsubstituted alkenyl, substituted or unsubstituted alkynyl, substituted or unsubstituted ester, carboxylic acid, substituted or unsubstituted acetyl, substituted or unsubstituted amido, substituted or unsubstituted carbamate, substituted or unsubstituted amino, substituted or unsubstituted alkoxy, substituted or unsubstituted alkoxycarbonyl, substituted or unsubstituted thioether, cyano, substituted or unsubstituted aryl, substituted or unsubstituted heteroaryl, substituted or unsubstituted cycloalkyl, substituted or unsubstituted heterocyclyl, and combinations thereof, wherein each substituted group has 1, 2 or 3 substituents;
V is N, or CH;
R^{d} is absent, halo, cyano, C₁₋₃ alkyl, or C₁₋₃ alkyloxy;
R^{c} is H, C₁-C₆ alkyl, OH, NH₂, fluoro or gem-difluoro;
n is 0, 1 or 2;
X³ is CH₂ (or CH if bonded to T through Z), NH (or N if bonded to T through Z);
R^{e} is H or C₁₋₃ alkyl;
Z² is a bond or C₁₋₃ alkylene; and
Z is a bond, C₁₋₃ alkylene, —(CH₂)₂C(O)NH—, —N(R^{b})—, where R^{b} is H, or C₁₋₃ alkyl; and
T is [1,2,3]triazolo[4,5-b]pyridinyl, pyrazolo[2,3-a]pyrimidinyl, 5,6-dihydropyrrolo[2,3-d]pyrimidinyl, pyrazolo[3,4-d]pyrimidinyl, phenyl, purinyl, pyrimidinyl, pyridinyl, pyrazinyl, pyridazinyl, pyrazolyl, tetrazolyl, 1,2,4-triazolyl, 1,2,3-triazolyl, imidazolyl, benzo[d]imidazolyl, pyrrolyl, pyrrolidinyl, isoxazolyl, isoindolyl, piperidinyl, oxetanyl, thiophenyl, morpholino, thiazolyl, pyridinyl-2-one, pyrazinyl-2-one, indazolyl, 4,5,6,7-tetrahydro-indazolyl, isoindolinyl-1,3-dione, 1,3,4-oxadiazolyl, 1,3,4-oxadiazolyl-2-one, benzo[d]isoxazolyl, benzo[d]isothiazolyl, 1,2,4-oxadiazolyl, 1,3,4-thiadiazolyl, 1,3-dihydro-2H-imidazolyl-2-one, phthalazinyl, isoquinolinyl, 2λ⁵-quinolinyl-2-one, 6,7-dihydro-4H-[1,2,3]triazolo[5,1-c][1,4]oxazinyl, 2,3-dihydro-4λ²-pyrido[3,2-b][1,4]oxazinyl, thiazolo[5,4-c]pyridinyl, or quinazolinyl, each optionally substituted with 1, 2 or 3 substituents independently chosen from OH, halo, cyano, substituted or unsubstituted aryl, substituted or unsubstituted heteroaryl, substituted or unsubstituted alkyl, haloalkyl, substituted or unsubstituted alkenyl, substituted or unsubstituted alkynyl, substituted or unsubstituted cycloalkyl, substituted or unsubstituted heterocyclyl, substituted or unsubstituted alkoxycarbonyl, hydroxycarbonyl, substituted or unsubstituted ester, substituted or unsubstituted amido, substituted or unsubstituted alkoxy, substituted or unsubstituted alkoxyaryl, substituted or unsubstituted alkoxyheteroaryl, substituted or unsubstituted thioether, substituted or unsubstituted amino, NH-aryl, and NH-heteroaryl, wherein each substituted group has 1, 2 or 3 substituents;
provided that when the compound is of Formula (Ia) then
i) when n is 1, R^{c} is absent, R^{d} is absent, Z is a bond, and M is 3-chloropyridin-2-yl or 3-methylpyridin-2-yl, then T is not unsubstituted pyrazolo[2,3-a]pyrimidinyl; methyl substituted pyrazolo[2,3-a]pyrimidinyl; unsubstituted [1,2,3]triazolo[4,5-b]pyridinyl; methyl substituted [1,2,3]triazolo[4,5-b]pyridinyl; or pyrazolyl substituted with a methyl group, a substituted carboxyl group, a substituted or unsubstituted tetrazolyl group, or a substituted or unsubstituted 1,3,4-oxadiazolyl-2-one group; or
ii) when n is 1, R^{c} is absent, R^{d} is absent, Z is a bond, and M is unsubstituted isoquinolinyl or methyl substituted pyrrolo[2,3-c]pyridinyl, then T is not a pyrazolyl substituted with methyl, substituted carboxyl or both methyl and substituted carboxyl; or
iii) when M is phenyl or pyridinyl, and Z is a bond, then T is not 5,5-dimethylisoxazolyl-4 (5H)-one or 4-alkoxy-5,5-dimethyl-4,5-dihydroisoxazolyl.

57. The compound of claim 56, of Formula (Ia), or a pharmaceutically acceptable salt thereof: 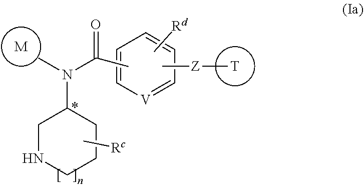
wherein
* denotes a stereocenter with (R) configuration;
R^{d} is absent, Cl, F, cyano, methyl, ethyl, methoxy or ethoxy;
R^{c} is absent, C₁-C₆ alkyl, OH, NH₂, fluoro or gem-difluoro;
n is 1 or 2;
Z is a bond, C₁₋₃ alkylene, —N(R^{b})—, where R^{b} is H, or C₁₋₃ alkyl; and
further wherein
i) when n is 1, R^{c} is absent, R^{d} is absent, Z is a bond, and M is 3-chloropyridin-2-yl or 3-methylpyridin-2-yl, then T is not unsubstituted pyrazolo[2,3-a]pyrimidinyl; methyl substituted pyrazolo[2,3-a]pyrimidinyl; unsubstituted [1,2,3]triazolo[4,5-b]pyridinyl; methyl substituted [1,2,3]triazolo[4,5-b]pyridinyl; or pyrazolyl substituted with a methyl group, a substituted carboxyl group, a substituted or unsubstituted tetrazolyl group, or a substituted or unsubstituted 1,3,4-oxadiazolyl-2-one group; or
ii) when n is 1, R^{c} is absent, R^{d} is absent, Z is a bond, and M is unsubstituted isoquinolinyl or methyl substituted pyrrolo[2,3-c]pyridinyl, then T is not a pyrazolyl substituted with methyl, substituted carboxyl or both methyl and substituted carboxyl; or
iii) when M is phenyl or pyridinyl, and Z is a bond, then T is not 5,5-dimethylisoxazolyl-4 (5H)-one or 4-alkoxy-5,5-dimethyl-4,5-dihydroisoxazolyl.

58. The compound of claim 57, wherein R^{c} is absent, methyl, ethyl, OH, NH₂, or fluoro and Z is a bond, methylene, —NH— or —N(CH₃)—.

59. The compound of claim 56, of Formula (Ib), or a pharmaceutically acceptable salt thereof: 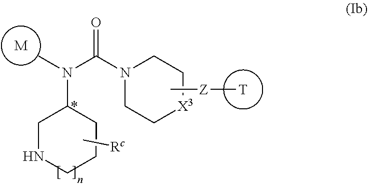
wherein
* denotes a stereocenter with (R) configuration;
X³ is CH₂ (or CH if bonded to T through Z), NH (or N if bonded to T through Z);
R^{c} is absent, C₁-C₆ alkyl, OH, NH₂, fluoro or gem-difluoro;
n is 1; and
Z is a bond, C₁₋₃ alkylene, —(CH₂)₂C(O)NH—, —N(R^{b})—, where R^{b} is H, or C₁₋₃ alkyl.

60. The compound of claim 59, wherein X³ is CH₂ (or CH if bonded to T through Z).

61. The compound of claim 59, wherein X³ is NH (or N if bonded to T through Z).

62. The compound of any one of claims 59 to 61, wherein Z is Z is a bond, methylene, —(CH₂)₂C(O)NH—, —NH— or —N(CH₃)—.

63. The compound of claim 56, of Formula (Ic), or a pharmaceutically acceptable salt thereof: 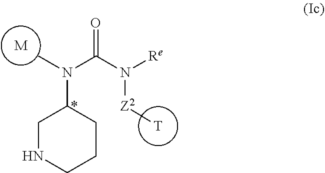
wherein
* denotes a stereocenter with (R) configuration;
R^{e} is H or C₁₋₃ alkyl; and
Z² is a bond or C₁₋₃ alkylene.

64. The compound of claim 63, wherein R^{e} is H, methyl, or isopropyl.

65. The compound of claim 63 or 64, wherein Z² is a bond.

66. The compound of claim 63 or 64, wherein Z² is methylene.

67. The compound of any one of claims 56 to 66, wherein M is 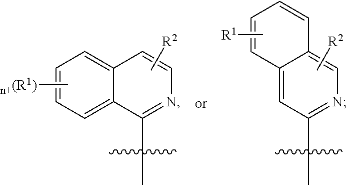
wherein
each R¹ is independently absent cyano; chloro; bromo; fluoro; acetyl; amido; carbamate; alkylcarbamate; pyrazinyl; tetrazolyl; hydroxycarbonyl; alkoxycarbonyl; pyridinyl-2-one; C₁₋₆ alkoxy; C₁₋₆ haloalkyl; C₁₋₆ cycloalkyl; C₁₋₆ alkyl optionally substituted with OH, pyridinyl, amido, or phenyl; C₂₋₆ alkenyl optionally substituted with amido, OH, pyrazolyl, pyridinyl, isoxazolyl, thiophenyl, alkyl substituted amido, hydroxyalkylamido, arylalkylamido, alkoxycarbonyl, or hydroxycarbonyl; C₂₋₆ alkynyl optionally substituted with OH; phenyl optionally substituted with OH, halo, hydroxyalkyl, sulfonyl, or alkylsulfonyl; pyrazolyl optionally substituted with C₁₋₆ alkyl, C₁₋₆ hydroxyalkyl, or acetamide; pyridinyl optionally substituted with amino; triazolyl optionally substituted with C₁₋₆ hydroxyalkyl; thiazolyl optionally substituted with C₁₋₆ alkyl; or 1,3,4-thiadiazolyl optionally substituted with C₁₋₆ alkyl;
n* is 0, 1 or 2;
R² is absent, halo, or C₁₋₆ alkyl, or cyano.

68. The compound of any one of claims 56 to 66, wherein M is 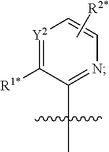
wherein
Y² is CH or N;
R^{1*} is absent; chloro; cyano; amido; alkylamido; allyl; alkoxycarbonyl; pyrazolyl; methylpyrazolyl; hydroxyethylpyrazolyl; isoxazolyl; 2,5-dihydro-pyrrolyl; thiophenyl; thieno[3,2-c]pyridinyl; C₁₋₆ alkyl; C₂₋₆ alkenyl optionally substituted with OH; C₂₋₆ alkenyl optionally substituted with OH;
R^{2*} is absent; C₁₋₆ alkyl; halo; pyrazolyl; C₂₋₆ alkenyl optionally substituted with phenyl, methylpyrazolyl, or pyridinyl.

69. The compound of any one of claims 56 to 66, wherein M is 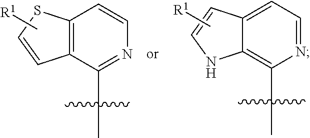
wherein
R¹ is absent, bromo, fluoro; chloro; amido; alkyl substituted amido; cyano; C₁₋₆ cycloalkyl, C₁₋₆ alkyl optionally substituted with phenyl or pyridinyl; C₂₋₆ alkenyl optionally substituted with OH, amido, alkyl substituted amido, or alkoxycarbonyl; C₂₋₆ alkynyl optionally substituted with OH, alkyl substituted amido, or pyridinyl; phenyl optionally substituted with one, two or three groups chosen from halo, OH, hydroxyalkyl, halohydroxyalkyl, cyano, CH₃O—, carboxyl, amino, amido, amidoalkyl, amidoalkoxy, alkyl substituted amido, phenyl substituted amido, hydroxyalkylamido, acetamido, aldehyde, acetyl, hydroxyalkylamido, sulfonylamino, alkylsulfonyl, isoxazolyl, pyrazolyl, and thiourea; pyridinyl optionally substituted with one or two groups chosen from C₁₋₆ alkyl, halo, amino, cyano, cyanoalkyl, haloalkyl, and alkoxy; 1,2,3-triazolyl optionally substituted with C₁₋₆ alkyl; thiazolyl optionally substituted with C₁₋₆ alkyl, hydroxyalkyl, alkoxycarbonyl, or hydroxycarbonyl; pyrazolyl optionally substituted with C₁₋₆ alkyl, hydroxyalkyl, pyridinyl, methylpyridinyl, or acetamido; benzo[d]isoxazolyl optionally substituted with amino; thiophenyl optionally substituted with hydroxyalkyl, cyano, or amido; pyrimidinyl optionally substituted with amino or alkylamino; pyridylcarbonyl; [1,2,4]triazolo[1,5-a]pyridinyl; pyrazinyl; pyridazinyl; pyridinyl-2-one; isoindolinyl-1-one; indolinyl; indolinyl-2-one; 1,3-dihydro-2H-benzo[d]imidazolyl-2-one; or indazolyl.

70. The compound of any one of claims 56 to 66, wherein T is a substituted or unsubstituted group chosen from [1,2,3]triazolo[4,5-b]pyridinyl, pyrazolo[2,3-a]pyrimidinyl, 5,6-dihydropyrrolo[2,3-d]pyrimidinyl, pyrazolo[3,4-d]pyrimidinyl, phenyl, purinyl, pyrimidinyl, pyridinyl, pyrazinyl, pyridazinyl, pyrazolyl, tetrazolyl, 1,2,4-triazolyl, 1,2,3-triazolyl, imidazolyl, benzo[d]imidazolyl, pyrrolyl, pyrrolidinyl, isoxazolyl, isoindolyl, piperidinyl, oxetanyl, thiophenyl, morpholino, thiazolyl, pyridinyl-2-one, pyrazinyl-2-one, indazolyl, 4,5,6,7-tetrahydro-indazolyl, isoindolinyl-1,3-dione, 1,3,4-oxadiazolyl, 1,3,4-oxadiazolyl-2-one, benzo[d]isoxazolyl, benzo[d]isothiazolyl, 1,2,4-oxadiazolyl, 1,3,4-thiadiazolyl, 1,3-dihydro-2H-imidazolyl-2-one, phthalazinyl, isoquinolinyl, 225-quinolinyl-2-one, 6,7-dihydro-4H-[1,2,3]triazolo[5,1-c][1,4]oxazinyl, 2,3-dihydro-4λ²-pyrido[3,2-b][1,4]oxazinyl, thiazolo[5,4-c]pyridinyl, and quinazolinyl.

71. The compound of claim 70, wherein T is [1,2,3]triazolo[4,5-b]pyridinyl optionally substituted with halo, haloalkyl, cyano, methyl, methoxy, ethyl, pyrazolyl, alkoxycarbonyl, hydroxycarbonyl, amido, or phenyl; 5,6-dihydropyrrolo[2,3-d]pyrimidinyl optionally substituted with methyl; isoxazolyl optionally substituted with methyl, haloalkyl, alkyoxycarbonyl, or amido; thiazolyl optionally substituted with methyl; pyrazolyl optionally substituted with C₁₋₆ alkyl, C₁₋₆ hydroxyalkyl, C₁₋₆ alkoxyalkyl, amino, phenyl, hydroxycarbonyl or any combination of two or three thereof; pyrimidinyl optionally substituted with amido, amino, or one or two methyl groups; 1,2,3-triazolyl optionally substituted with C₁₋₆ alkyl, C₁₋₆ hydroxyalkyl, C₁₋₆ alkoxyalkyl, halo, or any combination of two or three thereof; 1,2,4-triazolyl optionally substituted with methyl; phenyl optionally substituted with OH, pyrazolyl, pyridinyl, cyano, indazolyl, amidoindazolyl, methoxy, benzyloxy, C₂₋₆ alkynyl, or any combination of two or three thereof, and wherein the C₂₋₆ alkynyl is further substituted by OH, oxo, alkoxy, isoindolinyl-1,3-dione, morpholino, amino, hydroxyalkyl substituted amino, pyrrolidinyl substituted amino, hydroxypyrrolidinyl substituted amino, pyridinyl substituted amino, pyridinylmethyl substituted amino, or any combination of two or three thereof; pyridinyl optionally substituted with halo, methyl, amino pyridinyl or any combination of two or three thereof; pyrazolo[3,4-d]pyrimidinyl optionally substituted with phenoxyphenyl; imidazolyl optionally substituted phenyl, methyl, amino, or any combination of two or three thereof; pyrazinyl optionally substituted with OH, halo, cyano, amino, substituted amino, C₁₋₆ alkoxycarbonyl, C₁₋₆ alkyl, C₁₋₆ hydroxyalkyl, C₁₋₆ alkoxyalkyl, C₁₋₈ cycloalkyl, substituted C₁₋₈ cycloalkyl, C₁₋₃ alkyl substituted with cycloalkyl, C₁₋₃ alkyl substituted with substituted cycloalkyl, heterocyclyl, substituted heterocyclyl, C₁₋₃ alkyl substituted with heterocyclyl, C₁₋₃ alkyl substituted with substituted heterocyclyl, heteroaryl, substituted heteroaryl, C₁₋₃ alkyl substituted with heteroaryl, C₁₋₃ alkyl substituted with substituted heteroaryl, aryl, substituted aryl, C₁₋₃ alkyl substituted with aryl, C₁₋₃ alkyl substituted with substituted aryl, C₂₋₆ alkenyl, C₂₋₆ hydroxyalkenyl, C₂₋₆ alkoxyalkenyl, C₂₋₆ alkynyl, C₂₋₆ hydroxyalkynyl, C₂₋₆ alkoxyalkynyl or any combination of two or three thereof; tetrazolyl optionally substituted with methyl, ethyl, or haloalkyl; 1,3,4-oxadiazolyl optionally substituted with C₁₋₆ alkyl, C₃₋₆ cycloalkyl; 1,3,4-thiadiazolyl optionally substituted with C₁₋₆ alkyl, C₁₋₆ hydroxyalkyl, C₁₋₆ alkoxyalkyl, C₃₋₆ cycloalkyl, CD₃, OH; pyridinyl-2-one optionally substituted with methyl; 1,3-dihydro-2H-imidazolyl-2-one optionally substituted with methyl; 1,3,4-oxadiazolyl-2-one optionally substituted with methyl; pyrrolidinyl optionally substituted with methyl; benzo[d]isothiazolyl optionally substituted with methyl; 1,2,4-oxadiazolyl optionally substituted with C₁₋₆ alkyl, cyclopropyl or phenyl; morpholino optionally substituted with 1 or 2 C₁₋₆ alkyl groups; or pyridazinyl optionally substituted with halo.

72. The compound of Formula Ia, and pharmaceutically acceptable salts thereof, 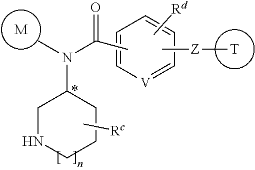
wherein
* denotes a stereocenter with (R) configuration;
wherein M is isoquinolinyl or a 9 membered fused bicyclic heteroaryl ring having 2 heteroatoms selected independently from nitrogen and sulfur, the isoquinolinyl or 9 membered ring optionally substituted with 1 to 2 substituents independently selected from pyridinyl, C₁₋₃ alkyl, C₁₋₃ alkoxy, fluoro or chloro; V is CH; n is 1; R^{c} is absent; R^{d} is absent, C₁₋₃ alkyl, C₁₋₃ alkoxy, fluoro or chloro; Z is a bond; and T is a 5 membered heteroaryl or a 9 membered fused bicyclic ring each 5 or 9 membered ring having 3-4 heteroatoms selected independently from nitrogen and sulfur and each 5 or 9 membered ring optionally substituted with 1 to 2 substituents selected independently from C₁₋₃ alkyl, C(D)₃, C₁₋₃ alkoxy, or fluoro.

73. The compound of claim 72, wherein M is thieno[3,2-c]pyridinyl or isoquinolinyl substituted with 1 to 2 substituents independently selected from pyridinyl, methyl, fluoro or chloro; T is a 9 membered fused bicyclic ring having 4 nitrogens and is optionally substituted with 1 to 2 substituents selected independently from fluoro, C₁₋₃ alkyl, C(D)₃.

74. The compound of claim 72, wherein M is thieno[3,2-c]pyridinyl or isoquinolinyl substituted with 1 to 2 substituents independently selected from pyridinyl, methyl fluoro, or chloro; and T is a 5 membered heteroaryl having 3 heteroatoms selected independently from nitrogen and sulfur optionally substituted with 1 substituent selected independently from fluoro, C₁₋₃ alkyl, C(D)₃.

75. The compound of claim 72, wherein M is thieno[3,2-c]pyridin-4-yl or isoquinolin-1-yl substituted with 1 to 2 substituents independently selected from pyridinyl, methyl or chloro; n is 1; R^{c} is absent; V is CH, R^{d} is absent, fluoro or chloro; Z is a bond; and T is [1,2,3]triazolo[4,5-b]pyridin-3-yl, thieno[3,2-c]pyridinyl, 1,2,3-triazol-4-yl or 1,3,4-thiadiazol-2-yl optionally substituted with 1 substituent selected independently from methyl or C(D)₃.

76. The compound of claim 72, wherein M is thieno[3,2-c]pyridin-4-yl substituted with pyridinyl, methyl or chloro; n is 1; R^{c} is absent; V is CH, R^{d} is absent, fluoro or chloro; Z is a bond; and T is [1,2,3]triazolo[4,5-b]pyridin-3-yl, thieno[3,2-c]pyridinyl, 1,2,3-triazol-4-yl or 1,3,4-thiadiazol-2-yl optionally substituted with 1 substituent selected independently from methyl or C(D)₃.

77. The compound of claim 72, wherein M is thieno[3,2-c]pyridin-4-yl substituted with pyridinyl or methyl; n is 1; R^{c} is absent; V is CH, R^{d} is fluoro or chloro; Z is a bond; and T is [1,2,3]triazolo[4,5-b]pyridin-3-yl, thieno[3,2-c]pyridinyl, or 1,3,4-thiadiazol-2-yl optionally substituted with 1 substituent selected independently from methyl or C(D)₃.

78. A compound of Formula Ia 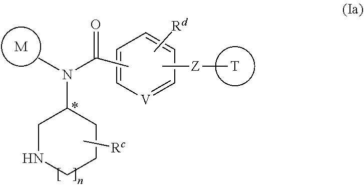
wherein
* denotes a stereocenter with (R) configuration;
M is isoquinolinyl substituted with 1 to 2 substituents independently selected from C₁₋₃ alkyl, C₁₋₃ alkoxy, fluoro or chloro; n is 1; R^{c} is absent; V is CH, R^{d} is absent, C₁₋₃ alkyl, C₁₋₃ alkoxy, fluoro or chloro; Z is a bond; and T is a 5 membered heteroaryl having 3 heteroatoms selected independently from nitrogen and sulfur optionally substituted with 1 to 2 substituents selected independently from C₁₋₃ alkyl, C(D)₃, C₁₋₃ alkoxy, or fluoro; and
further wherein T is not a pyrazolyl substituted with methyl, substituted carboxyl or both methyl and substituted carboxyl.

79. The compound of claim 72, wherein M is isoquinolinyl substituted with 1 to 2 substituents independently selected from methyl or chloro; n is 1; R^{c} is absent; V is CH, R^{d} is absent, methyl, fluoro or chloro; Z is a bond; and T is a 5 membered heteroaryl having 3 heteroatoms selected independently from nitrogen and sulfur optionally substituted with 1 substituent selected independently from methyl, C(D)₃, or fluoro.

80. The compound of claim 72, wherein M is isoquinolin-1-yl substituted with 1 to 2 substituents independently selected from methyl or chloro; n is 1; R^{c} is absent; V is CH, R^{d} is absent, fluoro or chloro; Z is a bond; and T is 1,2,3-triazol-4-yl or 1,3,4-thiadiazol-2-yl optionally substituted with 1 substituent selected independently from methyl or C(D)₃.

81. A compound selected from Table 1, and/or pharmaceutically acceptable salts thereof.

82. A compound selected from 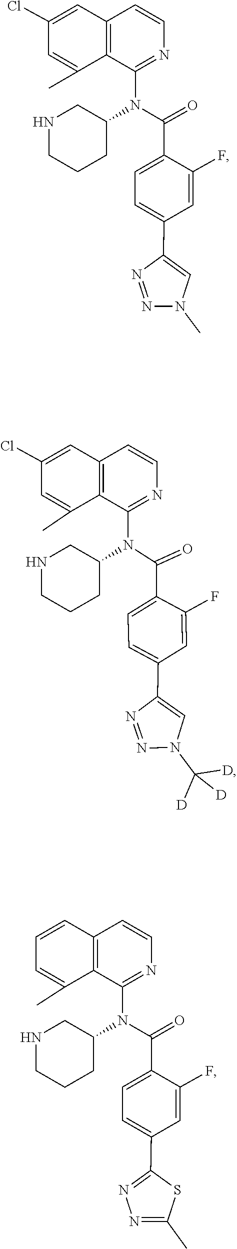 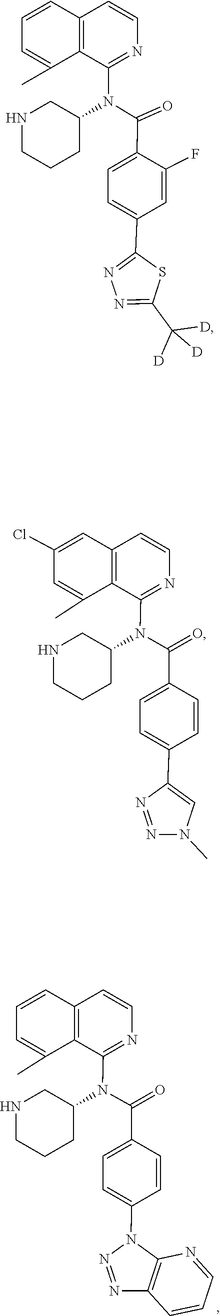 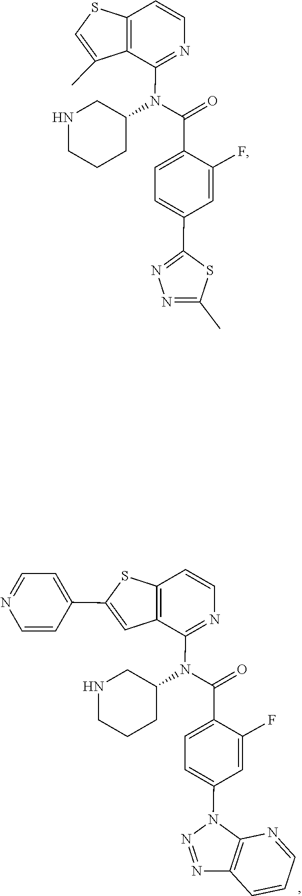 [SMILES: Cc1nnc(-c2ccc(C(=O)N(c3nccc4scc(C)c34)[C@@H]3CCCNC3)c(F)c2)s1 ; O=C(c1ccc(-n2nnc3cccnc32)cc1F)N(c1nccc2sc(-c3ccncc3)cc12)[C@@H]1CCCNC1]
and pharmaceutically acceptable salts thereof.

83. A compound selected from
N-(6-chloro-8-methyl-1-isoquinolyl)-2-fluoro-4-(1-methyltriazol-4-yl)-N-[(3R)-3-piperidyl] benzamide;
(R)—N-(6-chloro-8-methylisoquinolin-1-yl)-2-fluoro-4-(1-(methyl-d3)-1H-1,2,3-triazol-4-yl)-N-(piperidin-3-yl)benzamide;
(R)-2-fluoro-4-(5-methyl-1,3,4-thiadiazol-2-yl)-N-(8-methylisoquinolin-1-yl)-N-(piperidin-3-yl)benzamide;
(R)-2-fluoro-4-(5-(methyl-d3)-1,3,4-thiadiazol-2-yl)-N-(8-methylisoquinolin-1-yl)-N-(piperidin-3-yl)benzamide;
(R)—N-(6-chloro-8-methylisoquinolin-1-yl)-4-(1-methyl-1H-1,2,3-triazol-4-yl)-N-(piperidin-3-yl)benzamide;
(R)-4-(3H-[1,2,3]triazolo[4,5-b]pyridin-3-yl)-N-(8-methylisoquinolin-1-yl)-N-(piperidin-3-yl)benzamide;
(R)-2-fluoro-4-(5-methyl-1,3,4-thiadiazol-2-yl)-N-(3-methylthieno[3,2-c]pyridin-4-yl)-N-(piperidin-3-yl)benzamide;
(R)-4-(3H-[1,2,3]triazolo[4,5-b]pyridin-3-yl)-2-fluoro-N-(piperidin-3-yl)-N-(2-(pyridin-4-yl)thieno[3,2-c]pyridin-4-yl)benzamide; and pharmaceutically acceptable salts thereof.

84. A compound of any one of claims 56 to 83, wherein one or more hydrogen atoms attached to carbon atoms of the compound are replaced by deuterium atoms.

85. A pharmaceutical composition comprising a compound and/or a pharmaceutically acceptable salt of any one of claims 56 to 84 and a pharmaceutically acceptable excipient.

86. A compound of any one of claims 56 to 6784 for use as a medicament.

87. The compound of claim 86, wherein the compound is for use in treating a cancer associated with MYC activity.

88. Use of a compound of any one of claims 56 to 84 in the manufacture of a medicament for treating a disease in a subject in which MYC contributes to the pathology and/or symptoms of the disease.
